(19) United States
(12) Patent Application Publication
(10) Pub. No.: US 2025/0340574 A1
(43) Pub. Date: Nov. 6, 2025

(54) MODULATORS OF CYSTIC FIBROSIS TRANSMEMBRANE CONDUCTANCE REGULATOR

(71) Applicant: Vertex Pharmaceuticals Incorporated, Boston, MA (US)

(72) Inventors: Alexander Russell ABELA, Escondido, CA (US); Jeremy J. CLEMENS, San Diego, CA (US); Thomas CLEVELAND, San Marcos, CA (US); Christopher COOK, San Diego, CA (US); Timothy Richard COON, Carlsbad, CA (US); Sara Sabina HADIDA RUAH, La Jolla, CA (US); Haripada KHATUYA, San Diego, CA (US); Jason MCCARTNEY, Cardiff by the Sea, CA (US); Mark Thomas MILLER, Rancho Santa Fe, CA (US); Fabrice Jean Denis PIERRE, La Jolla, CA (US); Johnny UY, San Diego, CA (US); Jinglan ZHOU, San Diego, CA (US)

(21) Appl. No.: 18/854,322

(22) PCT Filed: Apr. 5, 2023
(86) PCT No.: PCT/US2023/017627

§ 371 Date: Oct. 4, 2024

Related U.S. Application Data
(60) Provisional application No. 63/328,097, filed on Apr. 6, 2022.
(60) Provisional application No. 63/393,405, filed on Jul. 29, 2022.

(51) Int. Cl. C07D 519/00; A61K 31/529; C07D 515/08

(52) U.S. Cl. CPC C07D 519/00; A61K 31/529; C07D 515/08

(57) ABSTRACT

This disclosure provides modulators of Cystic Fibrosis Transmembrane Conductance Regulator (CFTR), pharmaceutical compositions containing at least one such modulator, methods of treatment of CFTR mediated diseases, including cystic fibrosis, using such modulators and pharmaceutical compositions, combination pharmaceutical compositions and combination therapies employing those modulators, and processes and intermediates for making such modulators.

This application claims the benefit of priority to U.S. application 63/328,097 filed Apr. 6, 2022 and U.S. application 63/393,405 filed Jul. 29, 2022, the disclosures of which are incorporated herein by reference in their entireties.

The disclosure relates to modulators of Cystic Fibrosis Transmembrane Conductance Regulator (CFTR), pharmaceutical compositions containing the modulators, methods of treatment of CFTR mediated diseases, including cystic fibrosis, using such modulators, combination therapies and combination pharmaceutical compositions employing such modulators, and processes and intermediates for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of cystic fibrosis and other CFTR mediated diseases, and particularly the more severe forms of these diseases, are still needed.

One aspect of the disclosure provides novel compounds, including compounds of Formula I, including compounds of any of Formulae Ia, Ia(i), Ib, Ib(i), Ic, Ic(i), Id, Id(i), Ie, Ie(i), If, and If(i), tautomers thereof, deuterated derivatives of those compounds and tautomers, and pharmaceutically acceptable salts of any of the foregoing. Another aspect of the disclosure provides compounds of Formula II, Formula III, and Formula IV, tautomers thereof, deuterated derivatives of those compounds and tautomers, and pharmaceutically acceptable salts of any of the foregoing. A further aspect of the disclosure provides Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing.

Formula I encompasses compounds falling within the following structure:

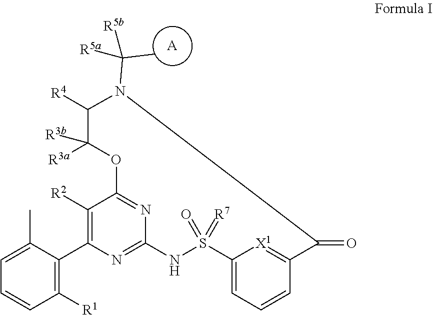

tautomers thereof, deuterated derivatives of those compounds and tautomers, and pharmaceutically acceptable salts of any of the foregoing, wherein:
Ring A is

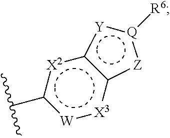

Q is selected from —C— and —N—;
W is selected from —CH—, —C(F)—, —C(CF₃)—, and —N—;
X¹, X², and X³ are each independently selected from —CH— and —N—;
Y is selected from —N—, —N(R^{y})—, —C(R^{y})—, and —O—, wherein
   R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CH—, —O—, —S—, —S(O)—, —S(O)₂—, —N—, and —N^{z}, wherein
   R^{z} is selected from hydrogen and C₁-C₈ alkyl;
R¹ is selected from: C₃-C₆ cycloalkyl, C₁-C₈ alkoxy, and C₁-C₈ alkyl (which may be optionally substituted with a group selected from C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   OC₃-C₇ cycloalkyl (which may be optionally substituted with 1 to 2 groups selected from C₁-C₈ alkyl, C₁-C₈ alkoxy, halogen, and C₁-C₈ haloalkyl);
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁶ is selected from halogen, 4- to 6-membered heterocyclyl, C₃-C₈ cycloalkyl (which may be optionally substituted with a group selected from C₁-C₈ alkyl, C₁-C₈ haloalkyl, and halogen); and C₁-C₈ alkyl (which may be optionally substituted with 1 to 2 groups independently selected from C₁-C₈ alkoxy, halogen, oxo, —OH, —NH₂, and —SO₂CH₃); and
R⁷ is selected from O, and NR, wherein
   R is selected from hydrogen and C₁-C₈ alkyl.

Formula I also includes compounds of:

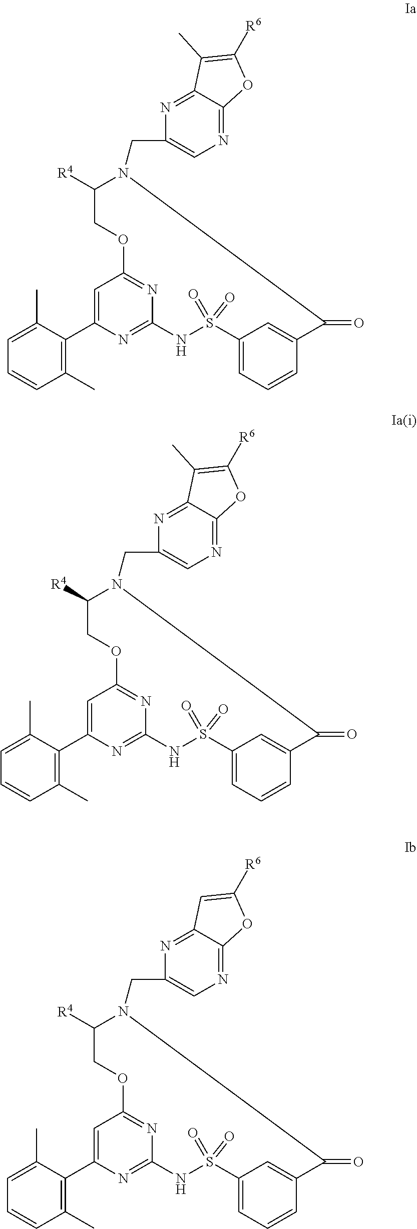

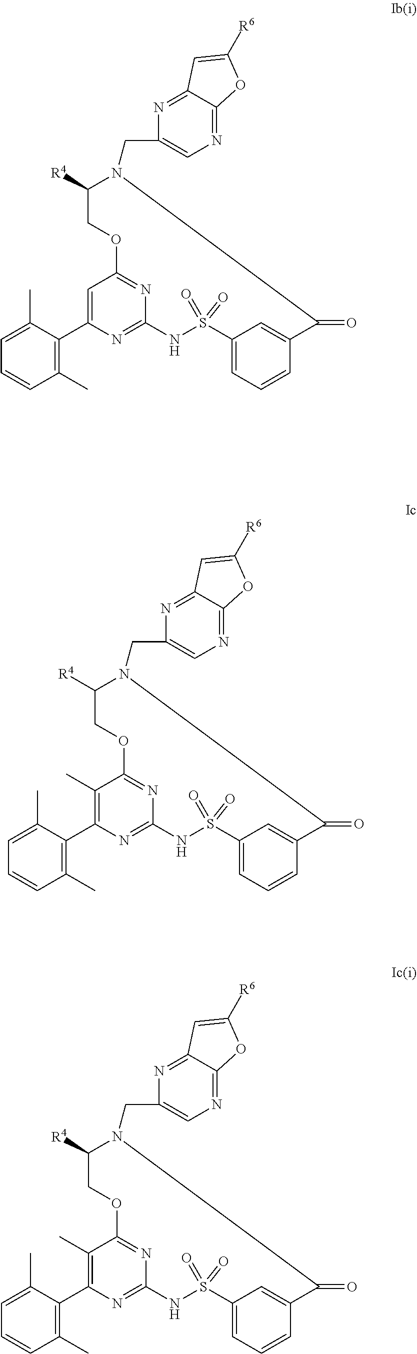

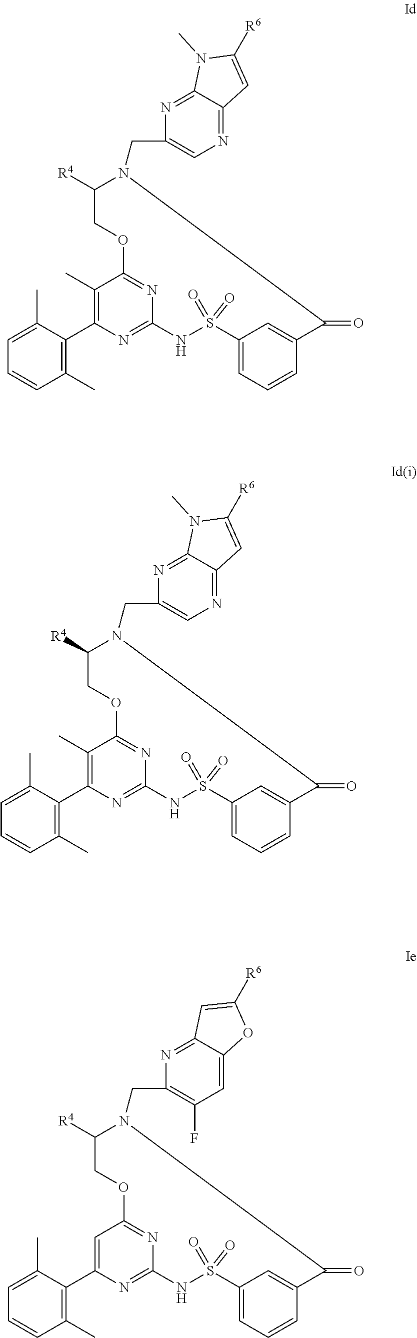

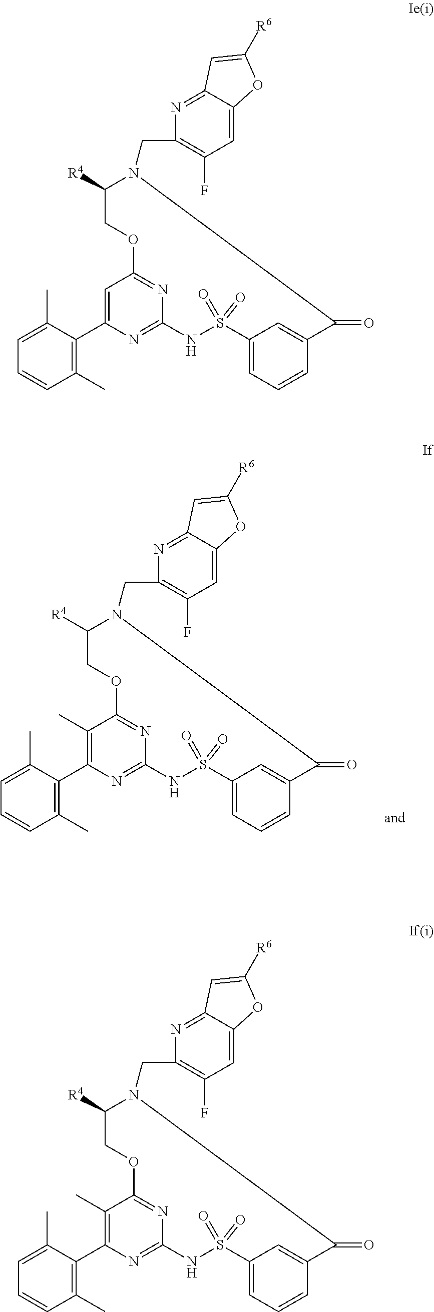

tautomers of those compounds, deuterated derivatives of those compounds and tautomers, and pharmaceutically acceptable salts of any of the foregoing, wherein all variables are as defined for Formula I.

In some embodiments, the compounds of Formula I are chosen from Compounds I-1 to I-265, tautomers of those compounds, deuterated derivatives of those compounds and tautomers, and pharmaceutically acceptable salts of any of the foregoing.

Formula II encompasses compounds falling within the following structure:

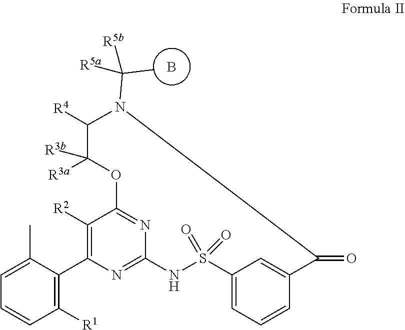

tautomers thereof, or deuterated derivatives of those compounds and tautomers, and pharmaceutically acceptable salts of any of the foregoing, wherein:
Ring B is a 6-membered heteroaryl, optionally substituted with 1 to 2 groups independently selected from
   halogen
   4- to 10-membered heterocyclyl (which may be optionally substituted with 1 to 3 groups independently selected from halogen, oxo, C₁-C₄ alkyl)
   N(R^{x})₂, wherein R^{x} is independently selected from hydrogen, C₁-C₄ alkyl, C₃-C₆ cycloalkyl (which may be optionally substituted with a group selected from halogen, C₁-C₄ haloalkyl, and C₁-C₄ alkyl)
   C₁-C₄ alkyl (optionally substituted with C₃-C₆ cycloalkyl (which may be further optionally substituted with a group selected from halogen, OH))
R¹ is selected from: C₃-C₆ cycloalkyl, C₁-C₆ alkoxy, and C₁-C₆ alkyl (which may be optionally substituted with a group selected from C₄-C₆ cycloalkyl, C₄-C₆ aryl, 4- to 6-membered heterocyclyl, and 4- to 6-membered heteroaryl);
R² is selected from: hydrogen, halogen, C₁-C₈ alkyl, C₁-C₄ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
   C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl).

In some embodiments, Ring B in the compounds, tautomer, deuterated derivative, or salt of Formula II is selected from:

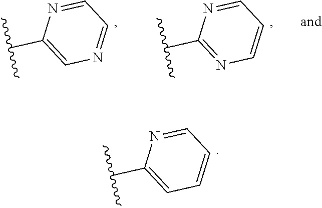 [SMILES: *c1cnccn1 ; *c1ncccn1 ; *c1ccccn1]

In some embodiments, the compounds of Formula II are chosen from Compounds II-1 to II-38, tautomers of those compounds, deuterated derivatives of those compounds and tautomers, and pharmaceutically acceptable salts of any of the foregoing.

Formula III encompasses compounds falling within the following structure:

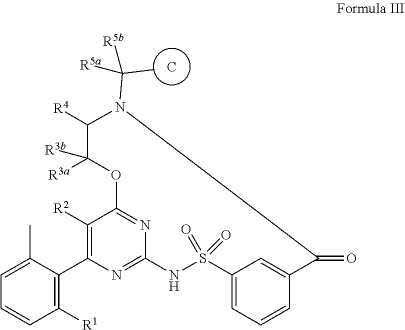

or a tautomer thereof, or a deuterated derivative of the compound or tautomer, or a pharmaceutically acceptable salt of any of the foregoing, wherein:
Ring C is selected from:

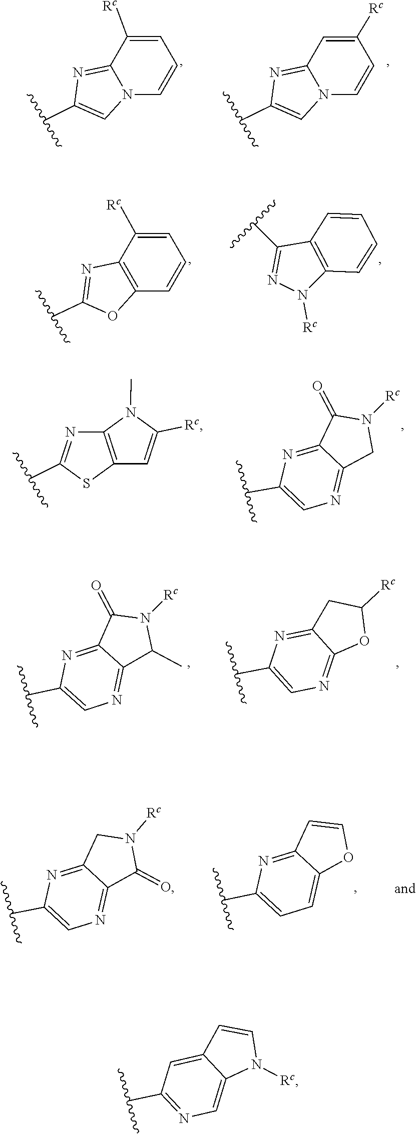

wherein
each R^{c} is independently selected from hydrogen, halogen, cyano, amino, C₁-C₄ alkyl (which may be optionally substituted with a group selected from —OH, halogen, and oxo), and C₃-C₆ alkenyl;
R¹ is selected from: C₃-C₆ cycloalkyl, C₁-C₆ alkoxy, and C₁-C₆ alkyl (which may be optionally substituted with a group selected from C₄-C₆ cycloalkyl, C₄-C₆ aryl, 4- to 6-membered heterocyclyl, and 4- to 6-membered heteroaryl);
R² is selected from: hydrogen, halogen, C₁-C₈ alkyl, C₁-C₄ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl).

In some embodiments, the compounds of Formula III are chosen from Compounds III-1 to III-25, tautomers of those compounds, deuterated derivatives of those compounds and tautomers, and pharmaceutically acceptable salts of any of the foregoing.

Formula IV encompasses compounds falling within the following structure:

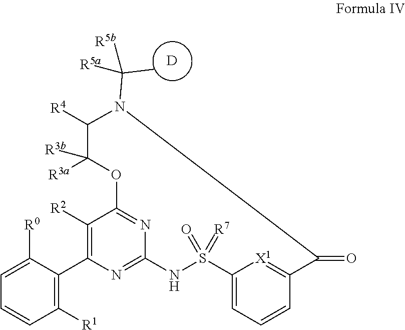

or a tautomer thereof, or a deuterated derivative of the compound or tautomer, or a pharmaceutically acceptable salt of any of the foregoing, wherein:
Ring D is

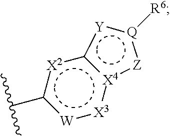

Q is selected from —C— and —N—;
W is selected from —CH—, —C(F)—, —C(CF₃)—, and —N—;
X¹, X², and X³ are each independently selected from —CH— and —N—;
X⁴ is selected from C and N;
Y is selected from —N—, —N(R^{y})—, —C(R^{y})—, and —O—, wherein
   R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CR^{z}—, —O—, —S—, —S(O)—, —S(O)₂—, —N—, and —N^{z}, wherein
   R^{z} is selected from hydrogen, halogen, and C₁-C₈ alkyl (which may be optionally substituted with C₁-C₈ alkoxy;
R⁰ is selected from C₁-C₂ alkyl;
R¹ is selected from: C₃-C₆ cycloalkyl, C₁-C₈ alkoxy, and C₁-C₈ alkyl (which may be optionally substituted with a group selected from C₁-C₈ alkoxy, C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₈ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   —OH
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   —OC₃-C₇ cycloalkyl (which may be optionally substituted with 1 to 2 groups selected from C₁-C₈ alkyl, C₁-C₈ alkoxy, halogen, and C₁-C₈ haloalkyl);
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl, and phenyl; or which may be optionally substituted with 1 to 3 halogen atoms);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
      R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
      R⁶ is selected from hydrogen, cyano, halogen, 4- to 6-membered heterocyclyl, 5- to 6-membered heteroaryl (which may be optionally substituted with 1 to 2 groups selected from C₁-C₈ alkyl), C₃-C₈ cycloalkyl (which may be optionally substituted with a group selected from C₁-C₈ alkyl, C₁-C₈ haloalkyl, and halogen), phenyl, and C₁-C₈ alkyl (which may be optionally substituted with 1 to 2 groups independently selected from C₁-C₈ alkoxy, C₁-C₈ haloalkyl, halogen, oxo, —OH, —NH₂, and —SO₂CH₃); and
      R⁷ is selected from O, and NR, wherein
         R is selected from hydrogen and C₁-C₈ alkyl.

In some embodiments, the compounds of Formula IV are chosen from Compounds IV-1 to IV-106, tautomers of those compounds, deuterated derivatives of those compounds and tautomers, and pharmaceutically acceptable salts of any of the foregoing.

Another aspect of the disclosure provides pharmaceutical compositions comprising at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier, which compositions may further include at least one additional active pharmaceutical ingredient. In some embodiments of the pharmaceutical compositions disclosed herein, the at least one additional active pharmaceutical ingredient is at least one other CFTR modulator. In some embodiments, the at least one other CFTR modulator is selected from CFTR potentiators and CFTR modulators.

Thus, another aspect of the disclosure provides methods of treating the CFTR-mediated disease cystic fibrosis comprising administering at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier, optionally as part of a pharmaceutical composition comprising at least one additional component, to a subject in need thereof. In some embodiments, the at least one additional active pharmaceutical ingredient in the methods of treating disclosed herein is at least one other CFTR modulator. In some embodiments, the at least one other CFTR modulator is selected from CFTR potentiators and CFTR correctors.

In certain embodiments, the pharmaceutical compositions of the disclosure comprise at least one compound chosen from compounds of Formula I, including compounds of any of Formulae Ia, Ia(i), Ib, Ib(i), Ic, Ic(i), Id, Id(i), Ie, Ie(i), If, and If(i), tautomers thereof, deuterated derivatives of those compounds and tautomers, and pharmaceutically acceptable salts of any of the foregoing. In certain embodiments, the pharmaceutical compositions of the disclosure comprise at least one compound chosen from compounds of Formula II, tautomers thereof, deuterated derivatives of those compounds and tautomers, and pharmaceutically acceptable salts of any of the foregoing. In certain embodiments, the pharmaceutical compositions of the disclosure comprise at least one compound chosen from compounds of Formula III, tautomers thereof, deuterated derivatives of those compounds and tautomers, and pharmaceutically acceptable salts of any of the foregoing. In certain embodiments, the pharmaceutical compositions of the disclosure comprise at least one compound chosen from compounds of Formula IV, tautomers thereof, deuterated derivatives of those compounds and tautomers, and pharmaceutically acceptable salts of any of the foregoing. In certain embodiments, the pharmaceutical compositions of the disclosure comprise at least one compound chosen from Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing.

In some embodiments, compositions comprising at least one compound chosen from compounds of Formula I, including compounds of any of Formulae Ia, Ia(i), Ib, Ib(i), Ic, Ic(i), Id, Id(i), Ie, Ie(i), If, If(i), II, ITT, and IV, tautomers thereof, deuterated derivatives of those compounds and tautomers, and pharmaceutically acceptable salts of any of the foregoing may optionally further comprise (a) at least one (i.e., one or more) compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo[d][1,3]dioxol-5-yl)cyclopropane carboxamido)-3-methylpyridin-2-yl)benzoic acid (lumacaftor), deuterated derivatives of tezacaftor and lumacaftor, and pharmaceutically acceptable salts of any of the foregoing; and/or (b) at least one (i.e., one or more) compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; deuterated derivatives of ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; and pharmaceutically acceptable salts of any of the foregoing.

In some embodiments, compositions comprising at least one compound chosen from Compounds I-1 to 1-265, Compounds II-1 to II-38, Compounds III-1 to 111-25, Compounds IV-1 to IV-106, tautomers thereof, deuterated derivatives of those compounds and tautomers, and pharmaceutically acceptable salts of any of the foregoing may optionally further comprise (a) at least one (i.e., one or more) compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo[d][1,3]dioxol-5-yl)cyclopropane carboxamido)-3-methylpyridin-2-yl)benzoic acid (lumacaftor), deuterated derivatives of tezacaftor and lumacaftor, and pharmaceutically acceptable salts of any of the foregoing; and/or (b) at least one (i.e., one or more) compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; deuterated derivatives of ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; and pharmaceutically acceptable salts of any of the foregoing.

Another aspect of the disclosure provides methods of treating the CFTR-mediated disease cystic fibrosis comprising administering to a patient in need thereof at least one compound chosen from the novel compounds disclosed herein, pharmaceutically acceptable salts thereof, and deuterated derivatives of any of the foregoing, and optionally further administering one or more additional CFTR modulating agents selected from tezacaftor, ivacaftor, and lumacaftor.

In a further aspect, compounds of the disclosure (e.g., compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing), and pharmaceutical compositions comprising those compounds, and optionally further comprising one or more CFTR modulating agents, are used in therapy or in the manufacture of a medicament. In some embodiments, the one or more additional CFTR modulating agents are selected from CFTR potentiators. In some embodiments, the one or more additional CFTR modulating agents are selected from CFTR correctors. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; and deuterated derivatives and pharmaceutically acceptable salts of any of the foregoing.

A further aspect of the disclosure provides intermediates and methods for making the compounds and compositions disclosed herein.

### Definitions

“Tezacaftor” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

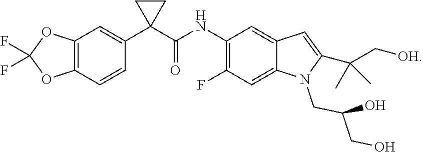 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative, a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each of which is incorporated herein by reference.

“Ivacaftor” as used throughout this disclosure refers to N-[2,4-bis(1,1-dimethylethyl)-5-hydroxyphenyl]-1,4-dihydro-4-oxoquinoline-3-carboxamide, which is depicted by the structure:

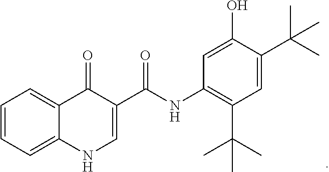 [SMILES: CC(C)(C)c1cc(C(C)(C)C)c(NC(=O)c2c[nH]c3ccccc3c2=O)cc1O]

Ivacaftor may also be in the form of a deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each of which is incorporated herein by reference.

In some embodiments, a deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

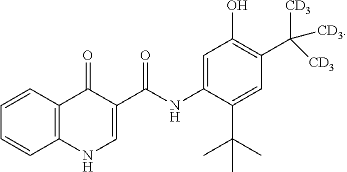 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of a further deuterated derivative, a pharmaceutically acceptable salt, or a pharmaceutically acceptable salt of a deuterated derivative. Deutivacaftor and methods of making and using deutivacaftor are disclosed in WO 2012/158885, WO 2014/078842, and U.S. Pat. No. 8,865,902, each of which is incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

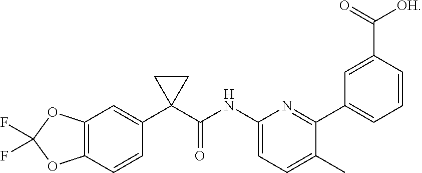 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using lumacaftor are disclosed in WO 2007/056341, WO 2009/073757, and WO 2009/076142, each of which is incorporated herein by reference.

(6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts are described in WO 2022/032068, incorporated herein by reference.

(6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol and deuterated derivatives and pharmaceutically acceptable salts are described in PCT/US2021/072475, incorporated herein by reference.

As used herein, the term “alkyl” refers to a saturated or partially saturated, branched, or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms), which may contain a double (alkenyl) or triple (alkynyl) bond between one or more sets of adjacent carbon atoms. Alkyl groups may be substituted or unsubstituted.

The term “aliphatic” or “aliphatic group,” as used herein, means a straight-chain (i.e., unbranched) or branched, substituted, or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic,” “carbocycle,” or “cycloalkyl”), that has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. In some embodiments, “cycloaliphatic” (or “carbocycle” or “cycloalkyl”) refers to a monocyclic C₃₋₈ hydrocarbon or bicyclic or tricyclic C₈₋₁₄ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof, such as (cycloalkyl)alkyl, (cycloalkenyl)alkyl, and (cycloalkyl)alkenyl. Suitable cycloaliphatic groups include cycloalkyl, bicyclic cycloalkyl (e.g., decalin), bridged bicycloalkyl such as norbornyl or [2.2.2]bicyclo-octyl, and bridged tricyclic such as adamantyl.

As used herein, the term “unsaturated” means that a moiety has one or more units of unsaturation.

As used herein, the term “pi bond” means a covalent bond formed by the p orbitals of adjacent atoms. Pi bonds exist where there is a multiple bond, i.e., a double or triple bond, between two atoms. For example, a carbon-carbon double bond consists of one pi bond, and a carbon-carbon triple bond consists of two pi bonds.

As used herein, the term “haloalkyl group” refers to an alkyl group substituted with one or more halogen atoms, e.g., fluoroalkyl, which refers to an alkyl group substituted with one or more fluorine atoms. In some embodiments, one carbon atom of the alkyl group is substituted with one or more halogen atoms. In some embodiments, each carbon atom of the alkyl group is substituted with one or more halogen atoms. In some embodiments, one or more carbon atoms of the alkyl group is a perhalo carbon atom (i.e., all hydrogen atoms of the alkyl group are substituted by halogen atoms). In some embodiments, each carbon atom of the alkyl group is a perhalo carbon atom. Non-limiting examples of fluoroalkyl include —CHF₂, —CH₂F, —CF₃, —CF₂—, and perhaloalkyl, such as —CF₂CF₃.

As used herein, the term “halogen” or “halo” means F, Cl, Br, or I.

As used herein, the terms “oxo” and “═O” refer to a substituent oxygen atom connected to another atom by a double bond.

The term “alkoxy,” as used herein, refers to an alkyl or cycloalkyl covalently bonded to an oxygen atom. Alkoxy groups may be substituted or unsubstituted.

As used herein, “cycloalkyl” refers to a cyclic, bicyclic, tricyclic, or polycyclic non-aromatic hydrocarbon groups having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, bicyclo[1.1.1]pentane, and spiro[2,3]hexane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems wherein at least one ring in the system is aromatic. An aryl group may be optionally substituted with one or more substituents. Non-limiting examples of aryl groups include phenyl, naphthyl, and 1,2,3,4-tetrahydronaphthalenyl.

The term “heteroatom” means one or more of oxygen, sulfur, nitrogen, phosphorus, or silicon (including any oxidized form of nitrogen, sulfur, phosphorus, or silicon; the quaternized form of any basic nitrogen; and a substitutable nitrogen of a heterocyclic ring, for example, N (as in 3,4-dihydro-2H-pyrrolyl), NH (as in pyrrolidinyl) or NR⁺ (as in N-substituted pyrrolidinyl)).

The term “heteroaliphatic,” as used herein, means aliphatic groups wherein one or two carbon atoms are independently replaced with one or more heteroatoms, for example, oxygen, sulfur, nitrogen, phosphorus, or silicon. Heteroaliphatic groups may be substituted or unsubstituted, branched or unbranched, cyclic or acyclic, and include “heterocycle,” “heterocyclyl,” “heterocycloaliphatic,” and “heterocyclic” groups.

The term “heteroaryl ring,” as used herein, refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) having a total of five to fourteen ring members, wherein at least one ring in the system is aromatic, at least one ring in the system contains one or more heteroatoms, and wherein each ring in the system contains three to seven ring members. Non-limiting examples of heteroaryl rings include pyridine, quinoline, indole, and indoline. A heteroaryl group may be optionally substituted with one or more substituents. In certain embodiments, the term “heteroaryl ring” encompasses heteroaryl rings with various oxidation states, such as heteroaryl rings containing N-oxides and sulfoxides. Non-limiting examples of such heteroaryl rings include pyrimidine N-oxides, quinoline N-oxides, thiophene S-oxides, and pyrimidine N-oxides.

As used herein, the term “heterocyclyl ring” refers to a non-aromatic hydrocarbon containing 3 to 12 atoms in a ring (such as, for example 3-10 atoms) comprising at least one ring atom that is a heteroatom, such as O, N, or S, and may include one or more unsaturated bonds. “Heterocyclyl” rings encompass monocyclic, bicyclic, tricyclic, polycyclic, bridged, fused, and spiro rings, including mono spiro and dispiro rings.

The bond designation “ [symbol] ” is intended to reflect the presence of an aromatic (i.e., conjugated) ring system. It will be appreciated that Ring A in Formula I comprises a six-membered aryl or heteroaryl ring fused to a five-membered aryl or heteroaryl ring.

It will be appreciated that certain compounds of this disclosure may exist as separate stereoisomers or enantiomers and/or mixtures of those stereoisomers or enantiomers. As used in the chemical structures disclosed herein, a “wedge” ( [symbol] ) or “hash” ( [symbol] ) bond to a stereogenic atom indicates a chiral center of known absolute stereochemistry (i.e., one stereoisomer). As used in the chemical structures disclosed herein, a “wavy” bond ( [symbol] ) to a stereogenic atom indicates a chiral center of unknown absolute stereochemistry (i.e., one stereoisomer). As used in the chemical structures disclosed herein, a “wavy” bond ( [symbol] ) to a double-bonded carbon indicates a mixture of E/Z isomers. As used in the chemical structures disclosed herein, a [symbol] (“straight”) bond to a stereogenic atom indicates where there is a mixture (e.g., a racemate or enrichment). As used herein, two [symbol] (“straight”) bonds to a double-bonded carbon indicates that the double bond possesses the E/Z stereochemistry as drawn. As used in the chemical structures disclosed herein, a

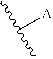

(a “wavy” line perpendicular to a “straight” bond to group “A”) indicates that group “A” is a substituent whose point of attachment is at the end of the bond that terminates at the “wavy” line. As used herein, a stereogenic atom that is notated with an (R) or (S) indicates the stereochemical designation of the stereogenic atom under the Cahn-Ingold-Prelog convention.

Certain compounds can exist as atropisomers. It will be appreciated that certain compounds of this disclosure may exist as separated atropisomers and/or mixtures of those atropisomers, i.e., a subclass of stereoisomers resulting from hindered rotation about single bonds or chirality axis and that can be isolated as separate chemical species. As used herein, a stereogenic unit that is notated with a (P) or (M) indicates the stereochemical designation of the stereogenic unit under the Cahn-Ingold-Prelog convention (basic terminology of stereochemistry, IUPAC Recommendations 1996, Pure & Appl. Chem., Vol 68, No. 12, pp. 2193-2222, 1996).

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

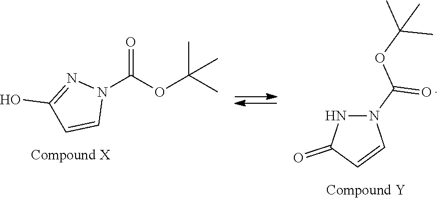

Unless otherwise stated, all tautomeric forms of the compounds of the disclosure are within the scope of the disclosure.

“Tert” and “t-” are used interchangeably and mean tertiary.

Compounds described herein may optionally be substituted with one or more substituents, such as are illustrated generally above, or as exemplified by particular classes, subclasses, and species of the disclosure. It will be appreciated that the phrase “optionally substituted” is used interchangeably with the phrase “substituted or unsubstituted.” “Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position. Combinations of substituents envisioned by this disclosure are preferably those that result in the formation of stable or chemically feasible compounds.

The term “stable,” as used herein, refers to compounds that are not substantially altered when subjected to conditions to allow for their production, detection, and preferably their recovery, purification, and use for one or more of the purposes disclosed herein.

The term “stable compounds,” as used herein, refers to compounds which possess sufficient stability to allow for their manufacture and which maintain the integrity of the compounds for a sufficient period of time to be useful for the purposes detailed herein (e.g., formulation into therapeutic products, intermediates for use in production of therapeutic compounds, isolatable or storable intermediates, and/or treating a disease or condition responsive to therapeutic agents).

In the compounds of this disclosure, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition.

As used herein, the term “derivative” refers to a collection of molecules having a chemical structure identical to a compound of this disclosure, except that one or more atoms of the molecule may have been substituted with another atom. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C, are within the scope of this disclosure. Such compounds are useful as, for example, analytical tools, probes in biological assays, or compounds with improved therapeutic profiles.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group. In chemical structures, deuterium may be represented as “D.”

As used herein, “CFTR” means cystic fibrosis transmembrane conductance regulator.

As used herein, the term “modulator” refers to a compound that increases the activity of a biological compound or molecule such as a protein.

As used herein, the term “CFTR modulator” refers to a compound that increases the activity of CFTR. The increase in activity resulting from a CFTR modulator includes, but is not limited to, compounds that correct, potentiate, stabilize, and/or amplify CFTR.

As used herein, the terms “corrector” and “CFTR corrector” are used interchangeably and refer to a compound that facilitates the processing and trafficking of CFTR to increase the amount of CFTR at the cell surface. The novel compounds disclosed herein are CFTR correctors. Tezacaftor and lumacaftor, and deuterated derivatives and pharmaceutically acceptable salts thereof as referenced herein, are correctors.

As used herein, the terms “potentiator” and “CFTR potentiator” refer to a compound that increases the channel activity of CFTR protein located at the cell surface, resulting in enhanced ion transport. Ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; as referenced herein, are CFTR potentiators. It will be appreciated that a description of a combination of compounds that includes a compound of the disclosure (e.g., compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing) will typically but not necessarily include a CFTR potentiator, such as, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; or a deuterated derivative or pharmaceutically acceptable salt of any of the foregoing. In addition, the combination will typically, but not necessarily, include only a single potentiator, but may include more than one corrector. Thus, in some embodiments, a combination of at least one compound of the disclosure (e.g., a compound selected from compounds of Formulae I, Ia, Ia(i), Ib, Ib(i), Ic, Ic(i), Id, Id(i), Ie, Ie(i), If, If(i), II, ITT, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing), will include a potentiator selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol; or deuterated derivatives or pharmaceutically acceptable salts thereof and may also include another CFTR corrector, such as, e.g., a corrector compound selected from tezacaftor, lumacaftor, and deuterated derivatives and pharmaceutically acceptable salts thereof. In some embodiments, such a combination may also include a CFTR potentiator enhancer.

As used herein, the term “CFTR potentiator enhancer,” “CFTR potentiation enhancer,” and “CFTR co-potentiator” are used interchangeably and refer to a compound that enhances CFTR potentiation.

The term “compound,” when referring to a compound of this disclosure, refers to a collection of molecules having an identical chemical structure, except that there may be isotopic variation among the constituent atoms of the molecules.

The phrase “a novel compound of the disclosure” refers to a compound chosen from compounds of any one of Formulae I, Ia, Ia(i), Ib, Ib(i), Ic, Ic(i), Id, Id(i), Ie, Ie(i), If, and If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing. Specifically excluded from the phrase “a novel compound of the disclosure is any compound disclosed in PCT/US2021/053858 (incorporated herein by reference).

The term “at least one compound selected from,” as used herein, refers to the selection of one or more of the compounds from a specified group. “Selected from” and “chosen from” may be used interchangeably herein.

A reference to “Compounds I-1 to I-265” herein is intended to represent a reference to each of Compounds 1 through 264 encompassed by Formula I, individually or as a group. Similarly, a reference to “Compounds II-1 to II-38” refers to Compounds 1 through 38 encompassed by Formula II, either as a group or each compound individually. A reference to “Compounds III-1 to III-24” refers to Compounds 1 through 24 encompassed by Formula III, either as a group or each compound individually. A reference to “Compounds IV-1 to IV-106” refers to Compounds 1 through 106 encompassed by Formula IV, either as a group or each compound individually. A reference to “Compounds I-1 to I-265, Compounds II-1 to II-38, Compounds III-1 to III-25, and Compounds IV-1 to IV-106” is intended to refer to compounds falling within each of Formulae I, II, III, and IV individually, or as three separate groups of compounds.

As used herein, the term “active pharmaceutical ingredient” or “therapeutic agent” (“API”) refers to a biologically active compound.

The terms “patient” and “subject” are used interchangeably and refer to an animal, including a human.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

It should be understood that references herein to methods of treatment (e.g., methods of treating a CFTR mediated disease or a method of treating cystic fibrosis) using one or more compounds of the disclosure optionally in combination with one or more additional CFTR modulating agents (e.g., a compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, tautomers thereof, deuterated derivatives of those compounds and tautomers, and pharmaceutically acceptable salts of any of the foregoing, optionally in combination with one or more additional CFTR modulating agents) should also be interpreted as references to:
one or more compounds of the disclosure (e.g., a compound chosen from compounds of Formulae I, Ia, Ia(i), Ib, Ib(i), Ic, Ic(i), Id, Id(i), Ie, Ie(i), If, and If(i), Compounds I-1 to I-265, Compounds II-1 to II-38, Compound III-1 to 111-25, Compounds IV-1 to IV-106, tautomers thereof, deuterated derivatives of those compounds and tautomers, and pharmaceutically acceptable salts of any of the foregoing, optionally in combination with one or more additional CFTR modulating agents) for use in methods of treating, e.g., cystic fibrosis, optionally in combination with one or more additional CFTR modulating agents; and/or
the use of one or more compounds of the disclosure (e.g., a compound chosen compounds of any of Formulae I, Ia, Ia(i), Ib, Ib(i), Ic, Ic(i), Id, Id(i), Ie, Ie(i), If, If(i), II, III, and IV, Compounds I-1 to 1-265, Compounds II-1 to II-38, Compound III-1 to 111-25, Compounds IV-1 to IV-106, tautomers thereof, deuterated derivatives of those compounds and tautomers, and pharmaceutically acceptable salts of any of the foregoing, optionally in combination with one or more additional CFTR modulating agents) in the manufacture of a medicament for treating, e.g., cystic fibrosis.

It should be also understood that references herein to methods of treatment (e.g., methods of treating a CFTR mediated disease or a method of treating cystic fibrosis) using a pharmaceutical composition of the disclosure (e.g., a pharmaceutical composition comprising at least one compound chosen from compounds of any of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, Compounds IV-1 to IV-106, tautomers thereof, deuterated derivatives of those compounds and tautomers, and pharmaceutically acceptable salts of any of the foregoing, and optionally further comprising one or more additional CFTR modulating agents) should also be interpreted as references to:
a pharmaceutical composition (e.g., a pharmaceutical composition comprising at least one compound chosen from compounds of any of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, Compounds IV-1 to IV-106, tautomers thereof, deuterated derivatives of those compounds and tautomers, and pharmaceutically acceptable salts of any of the foregoing, and optionally further comprising one or more additional CFTR modulating agents) for use in methods of treating, e.g., cystic fibrosis; and/or
the use of a pharmaceutical composition (e.g., a pharmaceutical composition comprising at least one compound chosen from compounds of any of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, Compounds IV-1 to IV-106, tautomers thereof, deuterated derivatives of those compounds and tautomers, and pharmaceutically acceptable salts of any of the foregoing, and optionally further comprising one or more additional CFTR modulating agents) in the manufacture of a medicament for treating, e.g., cystic fibrosis.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

As used herein, the terms “about” and “approximately,” when used in connection with amounts, volumes, reaction times, reaction temperatures, etc. mean an acceptable error for a particular value as determined by one of ordinary skill in the art, which depends in part on how the value is measured or determined. In some embodiments, the terms “about” and “approximately” mean within 1, 2, 3, or 4 standard deviations. In certain embodiments, the terms “about” and “approximately” mean within 30%, 25%, 20%, 15%, 10%, 9%, 8%, 7%, 6%, 5%, 4%, 3%, 2%, 1%, 0.5%, 0.1%, or 0.05% of a given value or range. As used herein, the symbol “˜” appearing immediately before a numerical value has the same meaning as the terms “about” and “approximately.”

The term “at least one” refers to one or more.

As used herein, the term “solvent” refers to any liquid in which the product is at least partially soluble (solubility of product >1 g/L).

Non-limiting examples of suitable solvents that may be used in this disclosure include, for example, water (H₂O), methanol (MeOH), methylene chloride or dichloromethane (DCM; CH₂Cl₂), acetonitrile (MeCN; CH₃CN), N,N-dimethylformamide (DMF), dimethylsulfoxide (DMSO), methyl acetate (MeOAc), ethyl acetate (EtOAc), isopropyl acetate (IPAc), tert-butyl acetate (t-BuOAc), isopropyl alcohol (IPA), tetrahydrofuran (THF), 2-methyl tetrahydrofuran (2-MeTHF), methyl ethyl ketone (MEK), tert-butanol, diethyl ether (Et₂O), methyl tert-butyl ether (MTBE), 1,4-dioxane, and N-methylpyrrolidone (NMP).

As used herein, the term “ambient conditions” means room temperature, open air condition and uncontrolled humidity condition. As used herein, the term “room temperature” or “ambient temperature” means 15° C. to 30° C.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. In general, a genetic defect or mutation, or a change in the nucleotides in a gene, results in a mutation in the CFTR protein translated from that gene, or a frame shift(s).

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “F508del” refers to a mutant CFTR protein which is lacking the amino acid phenylalanine at position 508, or to a mutant CFTR gene which encodes for a CFTR protein lacking the amino acid phenylalanine at position 508.

The disclosure also provides processes for preparing salts of the compounds of the disclosure. A salt of a compound of this disclosure is formed between an acid and a basic group of the compound, such as an amino functional group, or a base and an acidic group of the compound, such as a carboxyl functional group. In some embodiments, the salt is a pharmaceutically acceptable salt.

The term “pharmaceutically acceptable,” as used herein, refers to a component that is, within the scope of sound medical judgment, suitable for use in contact with the tissues of humans and other mammals without undue toxicity, irritation, allergic response, and the like, and is commensurate with a reasonable benefit/risk ratio.

As used herein, the term “pharmaceutically acceptable salt” means any non-toxic salt that, upon administration to a recipient, is capable of providing, either directly or indirectly, a compound of this disclosure. Pharmaceutically acceptable salts of the compounds of this disclosure include those derived from suitable inorganic and organic acids and bases. A “pharmaceutically acceptable counterion” is an ionic portion of a salt that is not toxic when released from the salt upon administration to a recipient. One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound.

A “free base” form of a compound does not contain an ionically bonded salt. It is noted that the disclosed amounts of the compounds or their pharmaceutically acceptable salts thereof herein are based upon their free base form. For example, “10 mg of at least one compound chosen from Compound I and pharmaceutically acceptable salts thereof” includes 10 mg of Compound I and a concentration of a pharmaceutically acceptable salt of Compound I equivalent to 10 mg of Compound I.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | Triethiodide |
| Esylate | Pantothenate | |
| Fumarate | Phosphate/diphosphate | |
| Gluceptate | Poly galacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

### CFTR Modulator Compounds

In some embodiments, the disclosure provides compounds of Formula I:

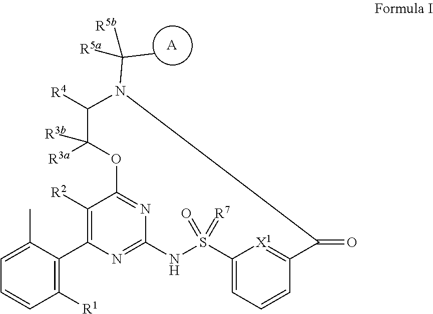

tautomers thereof, deuterated derivatives of those compounds and tautomers, and pharmaceutically acceptable salts of any of the foregoing, wherein:
Ring A is

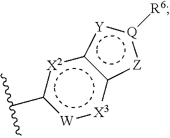

Q is selected from —C— and —N—;
W is selected from —CH—, —C(F)—, —C(CF₃)—, and —N—;
X¹, X², and X³ are each independently selected from —CH— and —N—;
Y is selected from —N—, —N(R^{y})—, —C(R^{y})—, and —O—, wherein
   R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CH—, —O—, —S—, —S(O)—, —S(O)₂—, —N—, and —N^{z}, wherein
   R^{z} is selected from hydrogen and C₁-C₈ alkyl;
R¹ is selected from: C₃-C₆ cycloalkyl, C₁-C₈ alkoxy, and C₁-C₈ alkyl (which may be optionally substituted with a group selected from C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   OC₃-C₇ cycloalkyl (which may be optionally substituted with 1 to 2 groups selected from C₁-C₈ alkyl, C₁-C₈ alkoxy, halogen, and C₁-C₈ haloalkyl);
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁶ is selected from halogen, 4- to 6-membered heterocyclyl, C₃-C₈ cycloalkyl (which may be optionally substituted with a group selected from C₁-C₈ alkyl, C₁-C₈ haloalkyl, and halogen); and C₁-C₈ alkyl (which may be optionally substituted with 1 to 2 groups independently selected from C₁-C₈ alkoxy, halogen, oxo, —OH, —NH₂, and —SO₂CH₃); and
R⁷ is selected from O, and NR, wherein
   R is selected from hydrogen and C₁-C₈ alkyl;
      with the proviso that the compound of Formula I is not selected from:

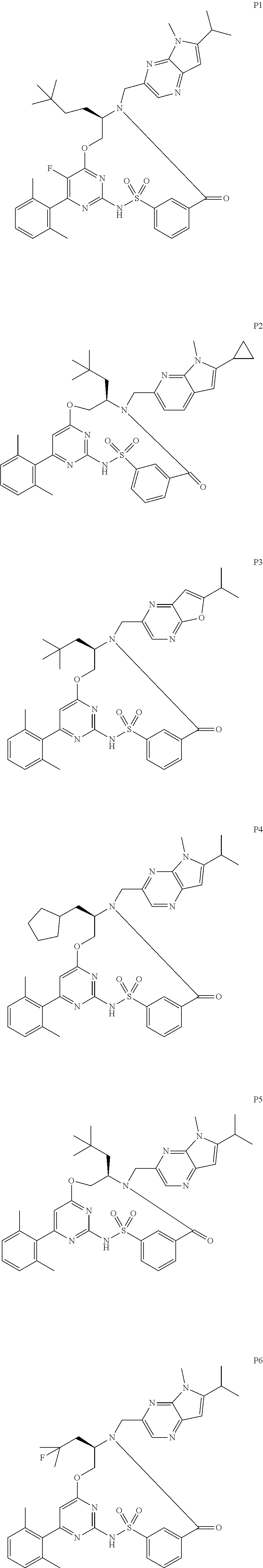

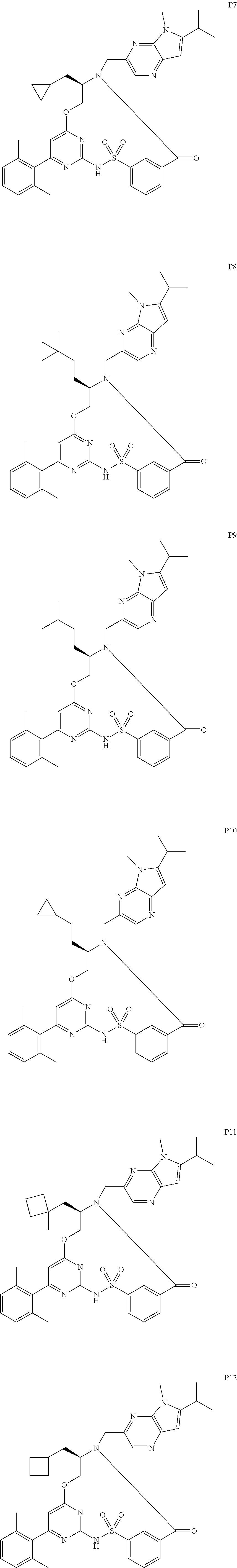

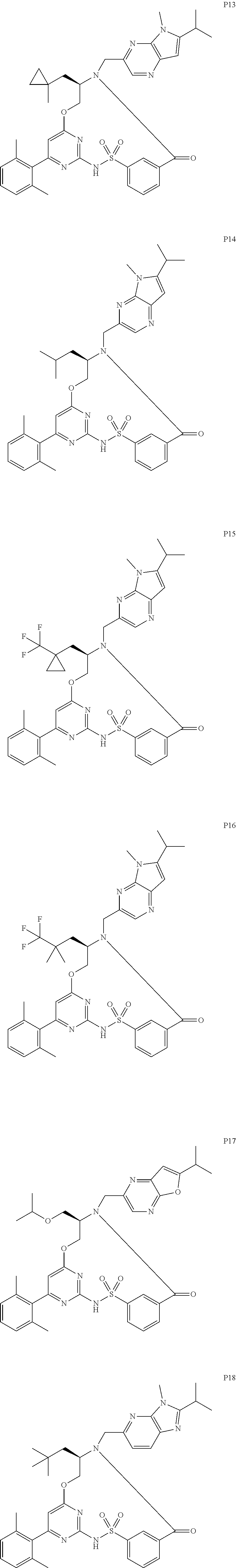

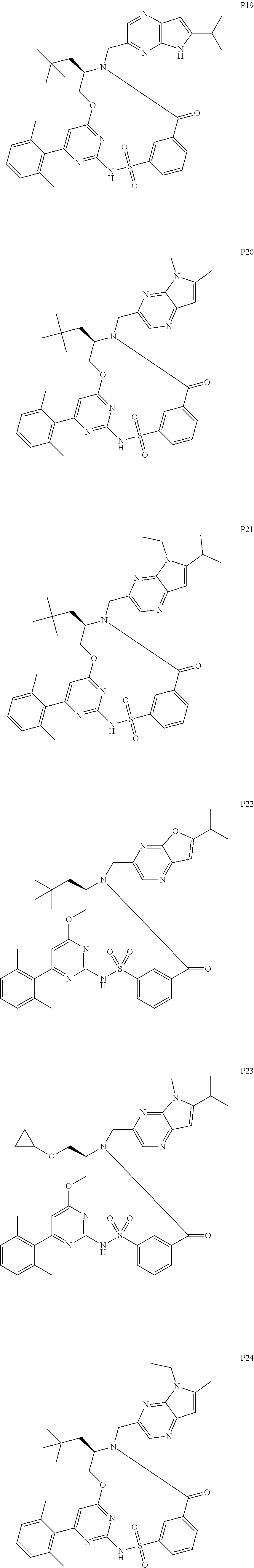

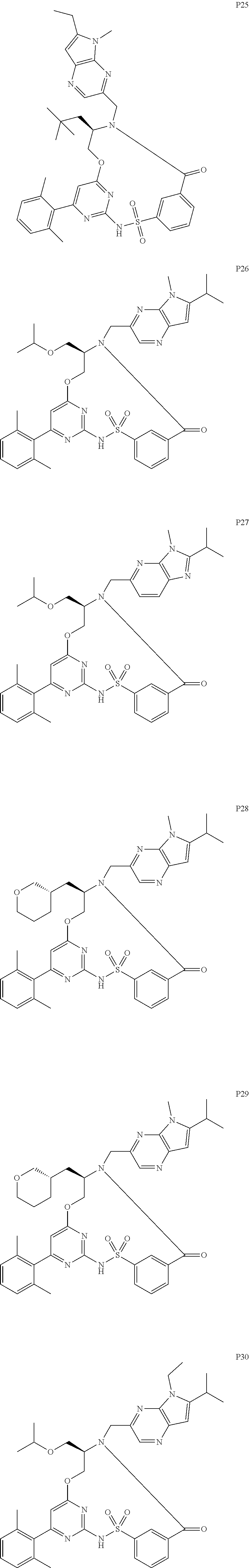

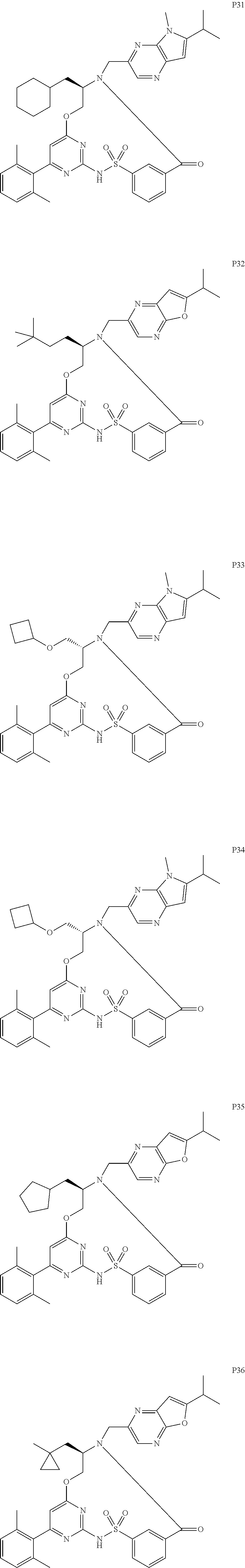

and tautomers, deuterated derivatives, and pharmaceutically acceptable salts thereof.

In some embodiments, Ring A in the compounds of Formula I is selected from:

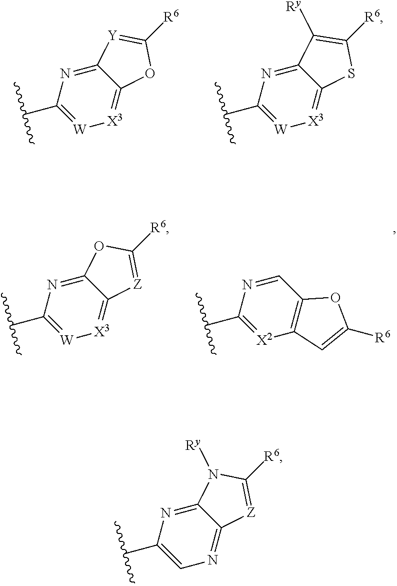

and wherein R⁶, W, X², X³, Y, R^{y}, and Z, are as defined above. In some embodiments, Ring A in the compounds of Formula I is selected from:

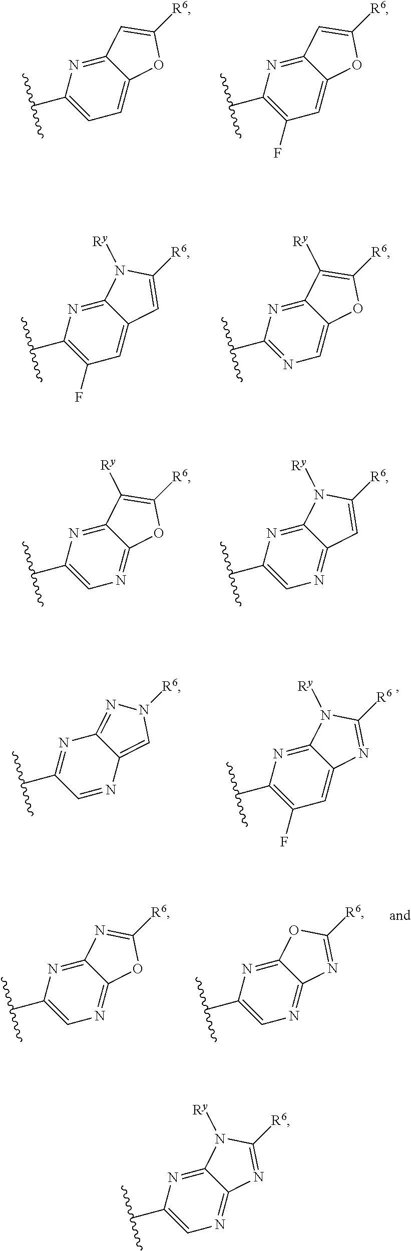

wherein R^{y} and R⁶ are as defined above.

In some embodiments, the compounds of Formula I are selected from compounds of

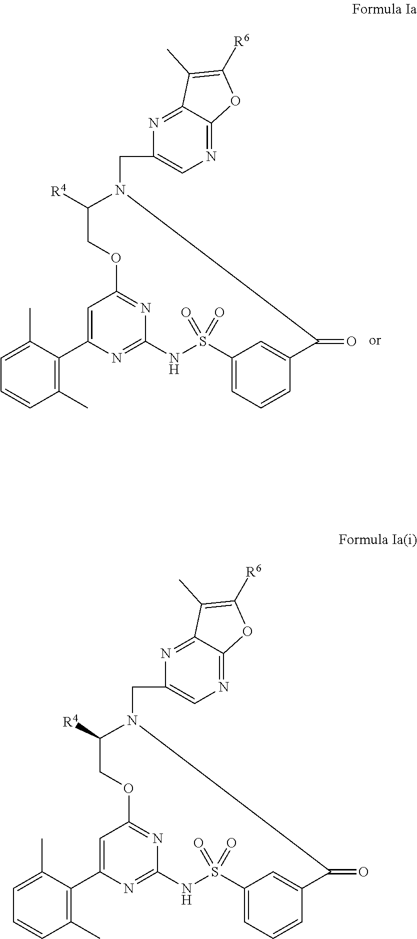

and tautomers thereof, deuterated derivatives of those compounds and tautomers, and pharmaceutically acceptable salts of any of the foregoing.

In some embodiments, the compounds of Formula I are selected from compounds of

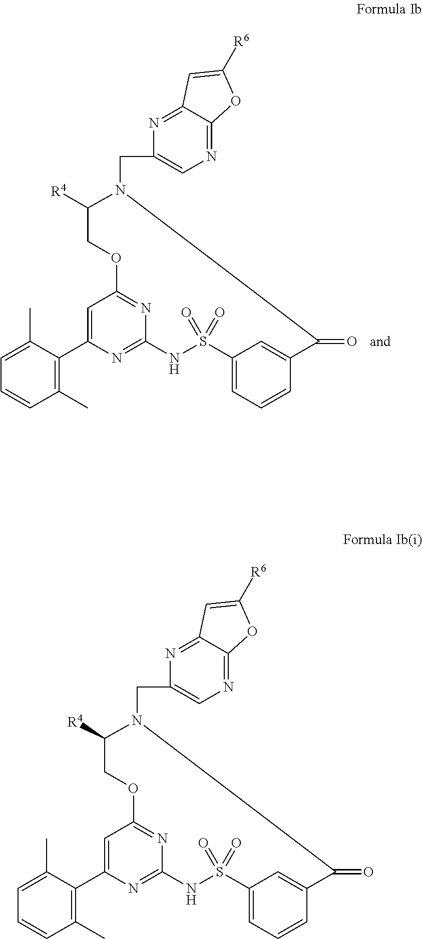

and tautomers thereof, deuterated derivatives of those compounds and tautomers, and pharmaceutically acceptable salts of any of the foregoing.

In some embodiments, the compounds of Formula I are selected from compounds of

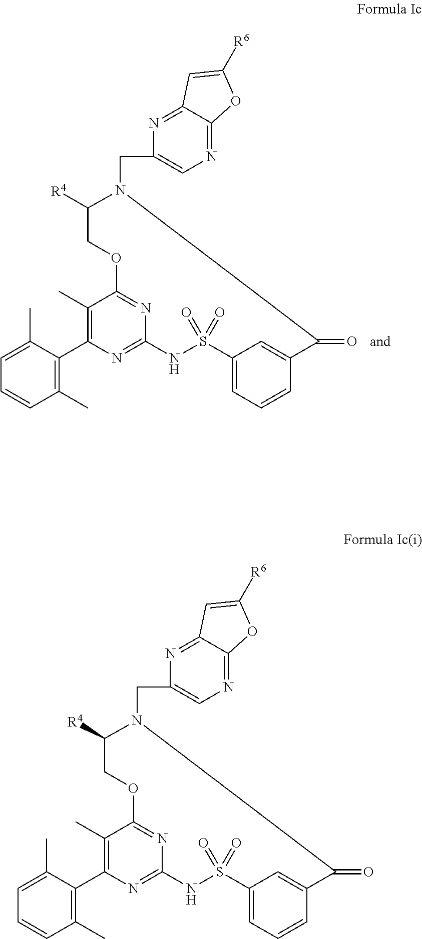

and tautomers thereof, deuterated derivatives of those compounds and tautomers, and pharmaceutically acceptable salts of any of the foregoing.

In some embodiments, the compounds of Formula I are selected from compounds of

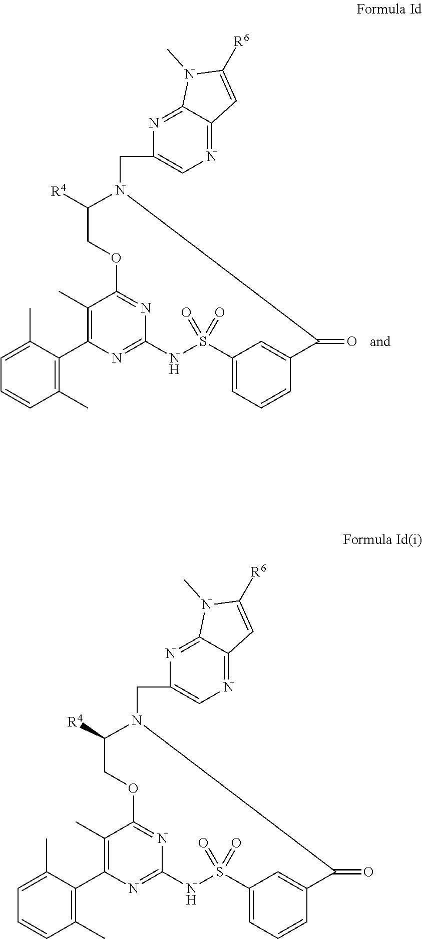

and tautomers thereof, deuterated derivatives of those compounds and tautomers, and pharmaceutically acceptable salts of any of the foregoing.

In some embodiments, the compounds of Formula I are selected from compounds of

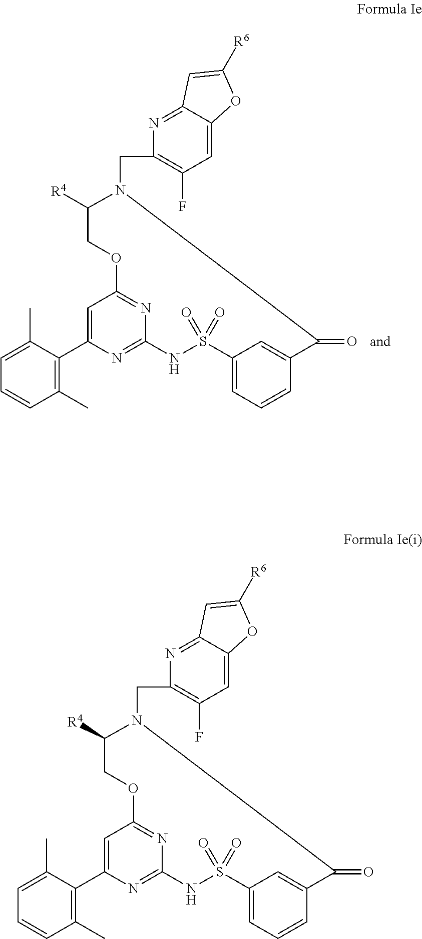

and tautomers thereof, deuterated derivatives of those compounds and tautomers, and pharmaceutically acceptable salts of any of the foregoing.

In some embodiments, the compounds of Formula I are selected from compounds of

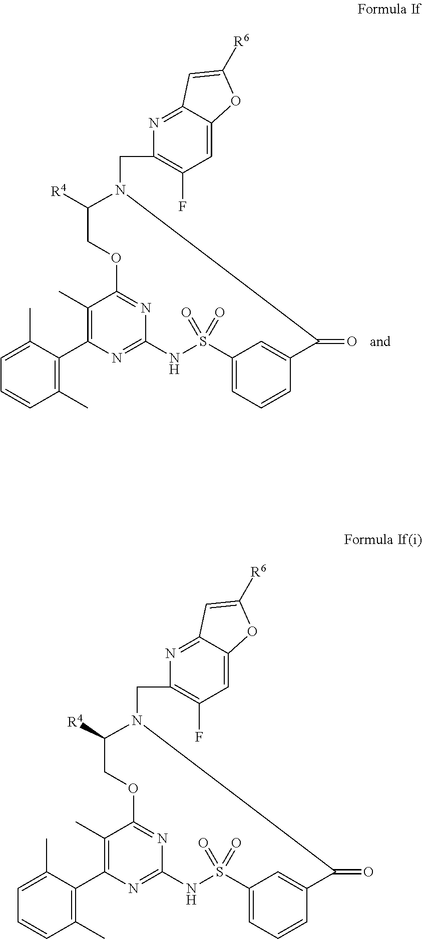

and tautomers thereof, deuterated derivatives of those compounds and tautomers, and pharmaceutically acceptable salts of any of the foregoing.

In some embodiments, R⁴ in the compounds of any of Formulae I, Ia, Ia(i), Ib, Ib(i), Ic, Ic(i), Id, Id(i), Ie, Ie(i), If, and If(i), is selected from C₁-C₆ alkyl optionally substituted with a group selected from halogen, haloalkyl, and C₁-C₄ alkoxy. In some embodiments, R⁴ in the compounds of any of Formulae I, Ia, Ia(i), Ib, Ib(i), Ic, Ic(i), Id, Id(i), Ie, Ie(i), If, and If(i), is selected from C₁-C₆ alkyl substituted with 1 to 2 groups independently selected from C₃-C₅ cycloalkyl (which may be optionally substituted with 1 to 2 groups selected from halogen, haloalkyl, and C₁-C₄ alkyl). In some embodiments, R⁴ in the compounds of any of Formulae I, Ia, Ia(i), Ib, Ib(i), Ic, Ic(i), Id, Id(i), Ie, Ie(i), If, and If(i), is selected from C₁-C₆ alkyl optionally substituted with phenyl (which may be optionally substituted with a group selected from C₁-C₄ alkyl, C₁-C₄ alkoxy, halogen, and haloalkyl). In some embodiments, R⁴ in the compounds of any of Formulae I, Ia, Ia(i), Ib, Ib(i), Ic, Ic(i), Id, Id(i), Ie, Ie(i), If, and If(i), is selected from C₁-C₆ alkyl substituted with a 4- to 6-membered heterocyclyl (which may be optionally substituted with 1 to 2 groups independently selected from halogen, haloalkyl, and C₁-C₄ alkyl).

In some embodiments, R⁴ in the compounds of any of Formulae I, Ia, Ia(i), Ib, Ib(i), Ic, Ic(i), Id, Id(i), Ie, Ie(i), If, and If(i), is selected from

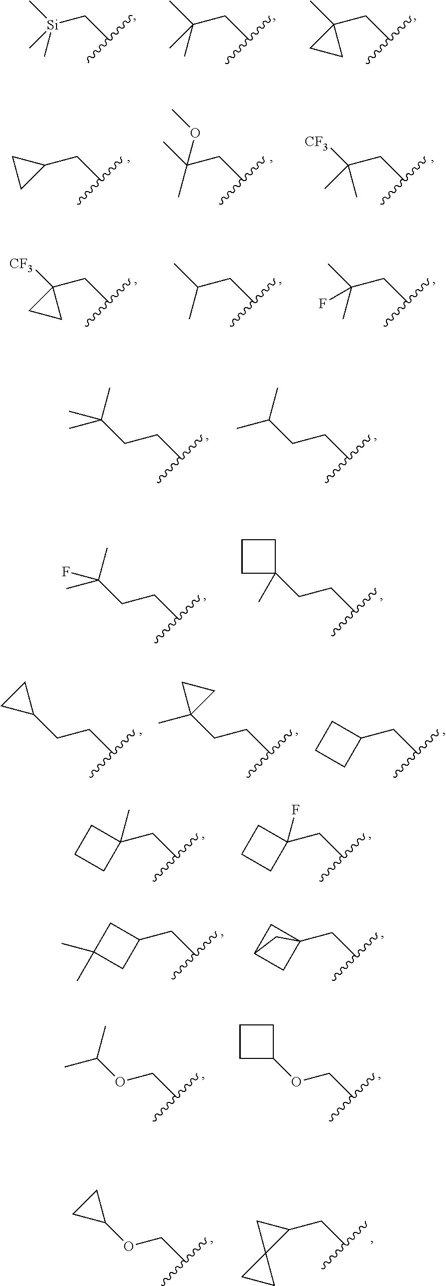

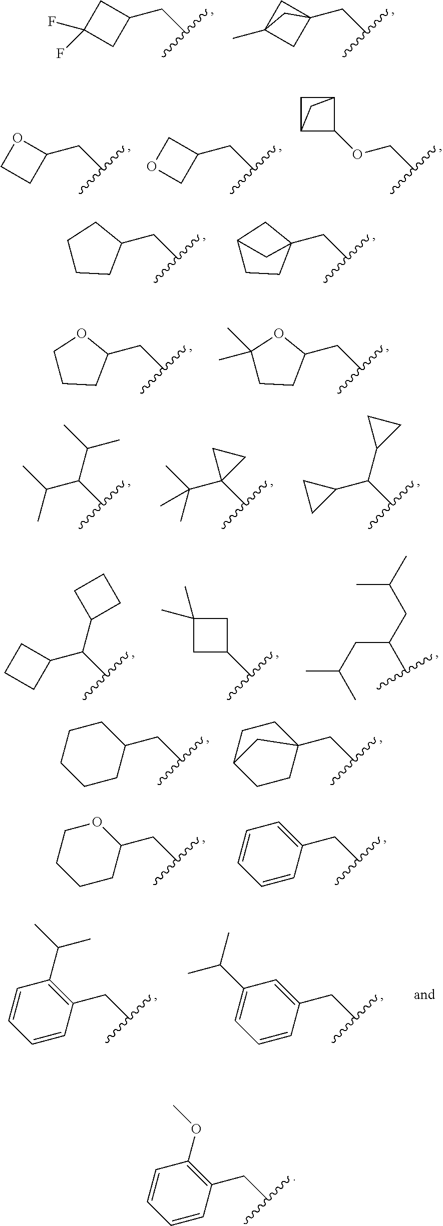

In some embodiments of Formula I, R^{y} is selected from bromine, chlorine, hydrogen, cyano, NH₂, butyl, cyclopropyl, CH₃, and CF₃. In some embodiments, R^{y} in compounds of Formula I, is selected from hydrogen, chlorine, amino, methyl, and butyl groups.

In some embodiments, R⁶ in the compounds of any of Formulae I, Ia, Ia(i), Ib, Ib(i), Ic, Ic(i), Id, Id(i), Ie, Ie(i), If, and If(i), is selected from C₃-C₈ cycloalkyl (which may be optionally substituted with a group selected from C₁-C₄ alkyl, haloalkyl, and halogen). In some embodiments, R⁶ in the compounds of any of Formulae I, Ia, Ia(i), Ib, Ib(i), Ic, Ic(i), Id, Id(i), Ie, Ie(i), If, and If(i), is selected from C₁-C₆ alkyl (which may be optionally substituted with 1 to 2 groups independently selected from C₁-C₄ alkoxy, halogen, —OH, oxo, —NH₂, and —SO₂CH₃). In some embodiments, R⁶ in the compounds of any of Formulae I, Ia, Ia(i), Ib, Ib(i), Ic, Ic(i), Id, Id(i), Ie, Ie(i), If, and If(i), is selected from:

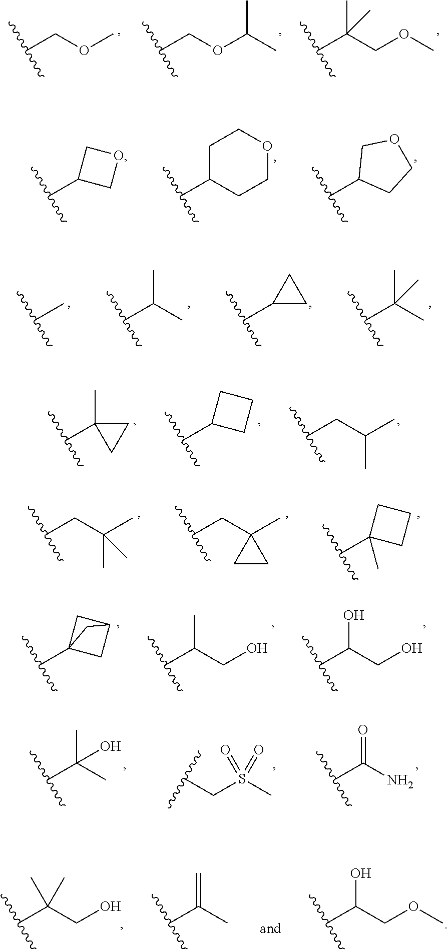

In some embodiments of Formula I, compounds are chosen from Compounds I-1 to I-265 and tautomers thereof, deuterated derivatives of the compounds and tautomers, and pharmaceutically acceptable salts of any of the foregoing.

| Compound Number | Structure |
|---|---|
| I-1 | 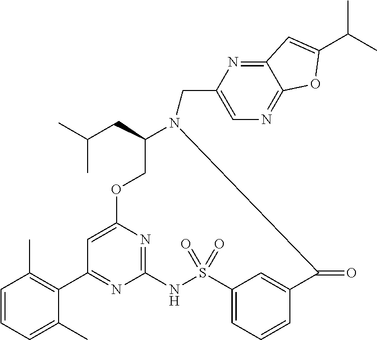 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)C)CO2] |
| I-2 | 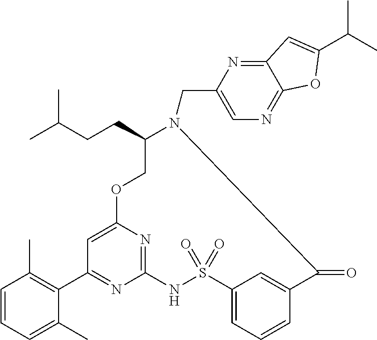 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)C)CO2] |
| I-3 | 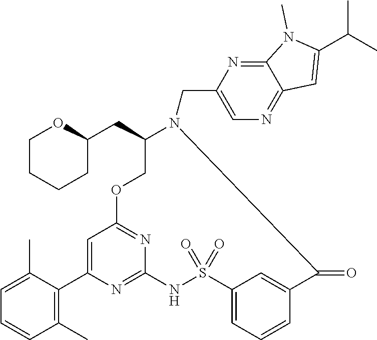 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCCO1)CO2] |
| I-4 | 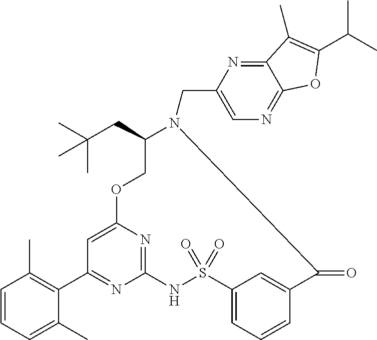 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)C)CO2] |
| I-5 | 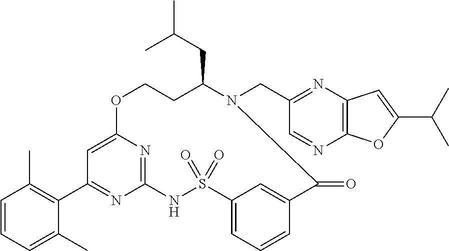 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)C)CCO2] |
| I-6 | 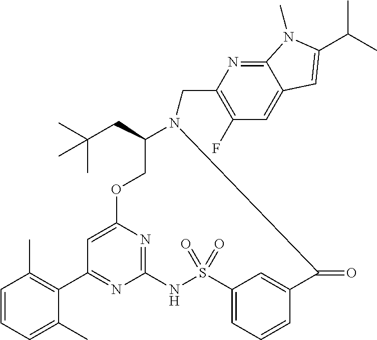 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC(C)(C)C)CO2] |
| I-7 | 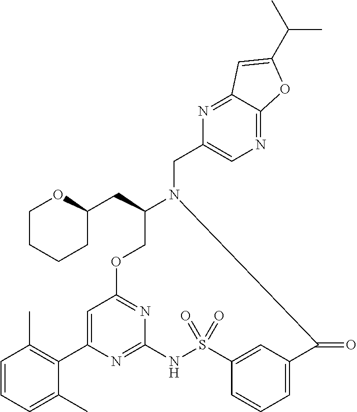 [SMILES: Cc1cccc(C)c1-c1cc2nc(n1)NS(=O)(=O)c1cccc(c1)C(=O)N(Cc1cnc3oc(C(C)C)cc3n1)[C@H](C[C@H]1CCCCO1)CO2] |
| I-8 | 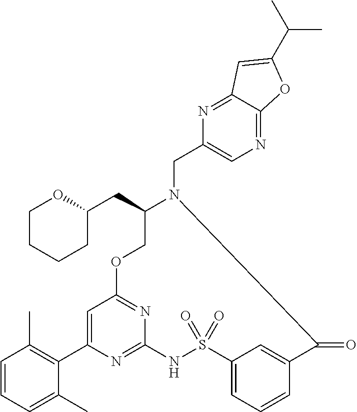 [SMILES: Cc1cccc(C)c1-c1cc2nc(n1)NS(=O)(=O)c1cccc(c1)C(=O)N(Cc1cnc3oc(C(C)C)cc3n1)[C@H](C[C@@H]1CCCCO1)CO2] |
| I-9 | 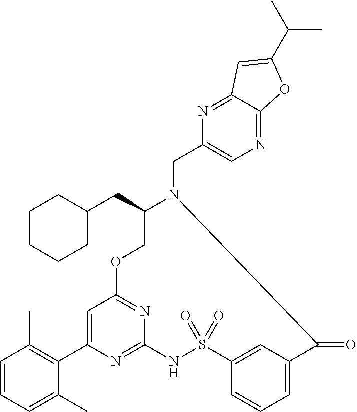 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCCC1)CO2] |
| I-10 | 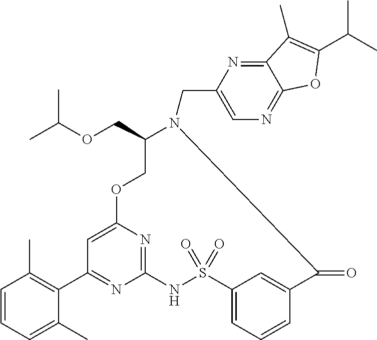 [SMILES: Cc1cccc(C)c1-c1cc2nc(n1)NS(=O)(=O)c1cccc(c1)C(=O)N(Cc1cnc3oc(C(C)C)c(C)c3n1)[C@H](COC(C)C)CO2] |
| I-11 | 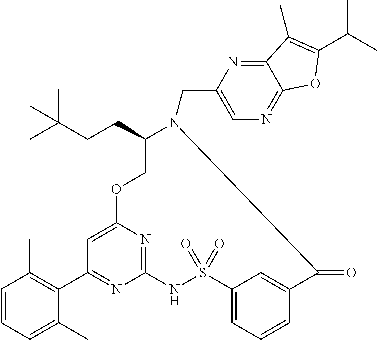 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)(C)C)CO2] |
| I-12 | 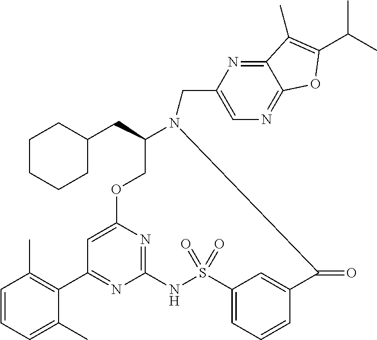 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCCC1)CO2] |
| I-13 | 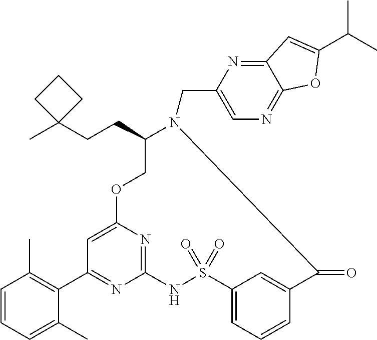 [SMILES: Cc1cccc(C)c1-c1cc2nc(n1)NS(=O)(=O)c1cccc(c1)C(=O)N(Cc1cnc3oc(C(C)C)cc3n1)[C@H](CCC1(C)CCC1)CO2] |
| I-14 | 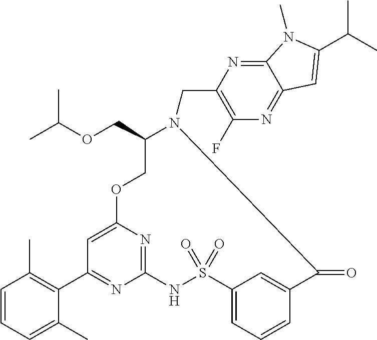 [SMILES: Cc1cccc(C)c1-c1cc2nc(n1)NS(=O)(=O)c1cccc(c1)C(=O)N(Cc1nc3c(cc(C(C)C)n3C)nc1F)[C@H](COC(C)C)CO2] |
| I-15 | 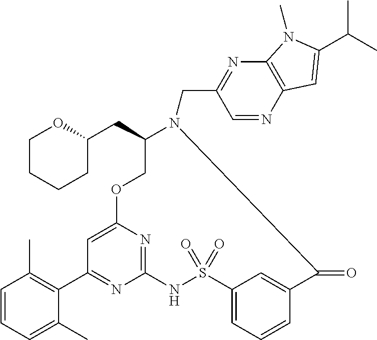 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCCO1)CO2] |
| I-16 | 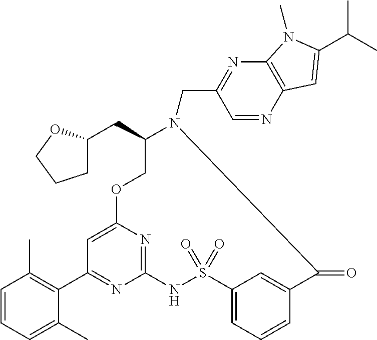 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCO1)CO2] |
| I-17 | 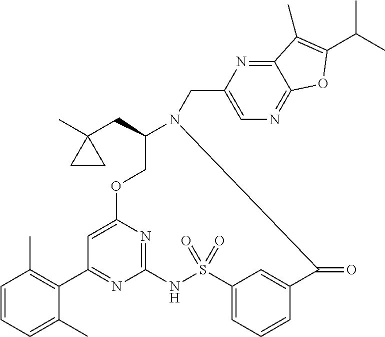 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1(C)CC1)CO2] |
| I-18 | 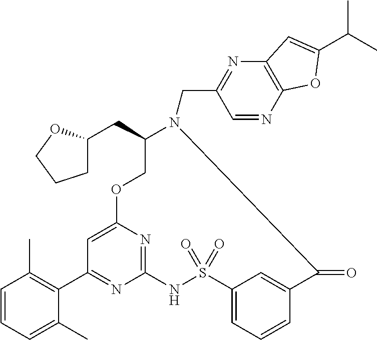 [SMILES: Cc1cccc(C)c1-c1cc2nc(n1)NS(=O)(=O)c1cccc(c1)C(=O)N(Cc1cnc3oc(C(C)C)cc3n1)[C@H](C[C@@H]1CCCO1)CO2] |
| I-19 | 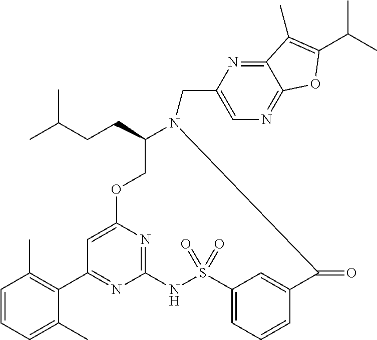 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)C)CO2] |
| I-20 | 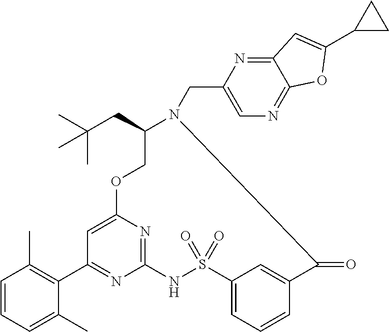 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)C)CO2] |
| I-21 | 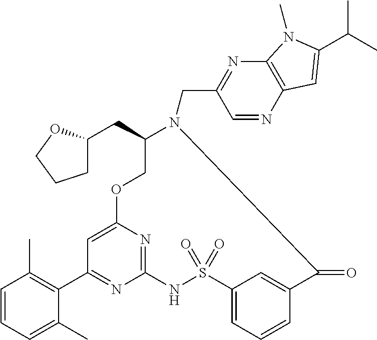 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCO1)CO2] |
| I-22 | 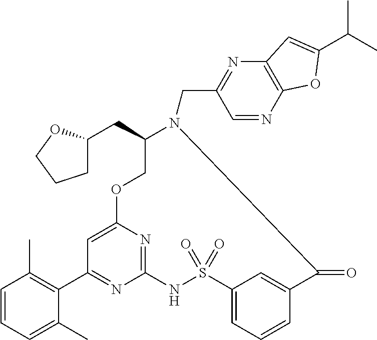 [SMILES: Cc1cccc(C)c1-c1cc2nc(n1)NS(=O)(=O)c1cccc(c1)C(=O)N(Cc1cnc3oc(C(C)C)cc3n1)[C@H](C[C@@H]1CCCO1)CO2] |
| I-23 | 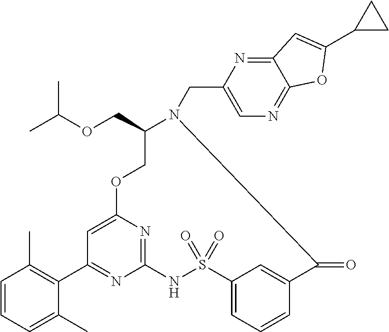 [SMILES: Cc1cccc(C)c1-c1cc2nc(n1)NS(=O)(=O)c1cccc(c1)C(=O)N(Cc1cnc3oc(C4CC4)cc3n1)[C@H](COC(C)C)CO2] |
| I-24 | 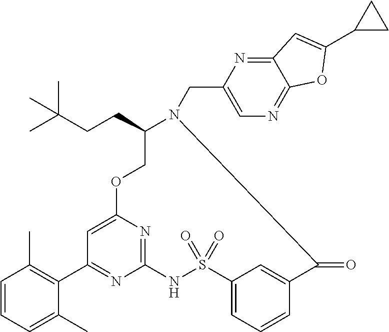 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)C)CO2] |
| I-25 | 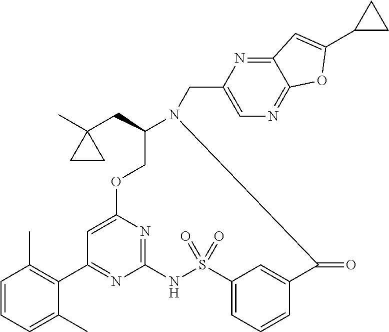 [SMILES: Cc1cccc(C)c1-c1cc2nc(n1)NS(=O)(=O)c1cccc(c1)C(=O)N(Cc1cnc3oc(C4CC4)cc3n1)[C@H](CC1(C)CC1)CO2] |
| I-26 | 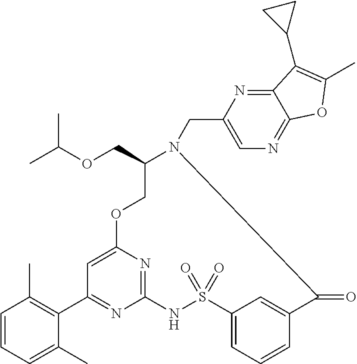 [SMILES: Cc1cccc(C)c1-c1cc2nc(n1)NS(=O)(=O)c1cccc(c1)C(=O)N(Cc1cnc3oc(C)c(C4CC4)c3n1)[C@H](COC(C)C)CO2] |
| I-27 | 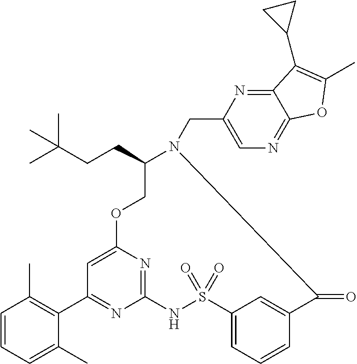 [SMILES: Cc1cccc(C)c1-c1cc2nc(n1)NS(=O)(=O)c1cccc(c1)C(=O)N(Cc1cnc3oc(C)c(C4CC4)c3n1)[C@H](CCC(C)(C)C)CO2] |
| I-28 | 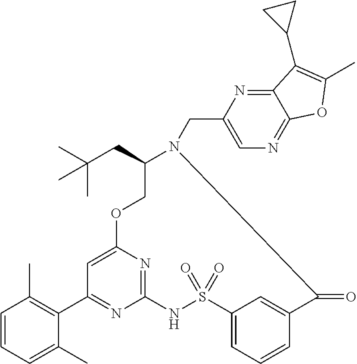 [SMILES: Cc1cccc(C)c1-c1cc2nc(n1)NS(=O)(=O)c1cccc(c1)C(=O)N(Cc1cnc3oc(C)c(C4CC4)c3n1)[C@H](CC(C)(C)C)CO2] |
| I-29 | 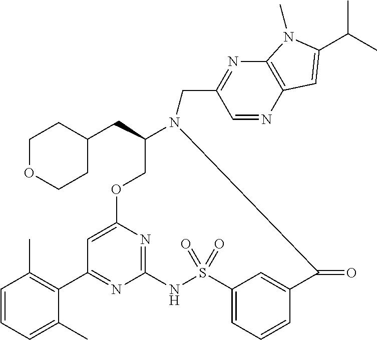 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCOCC1)CO2] |
| I-30 | 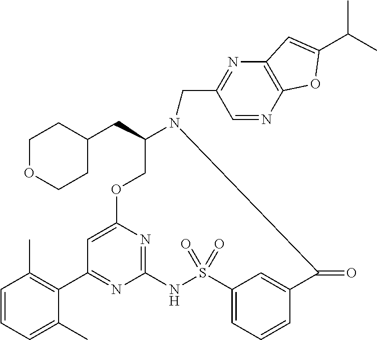 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCOCC1)CO2] |
| I-31 | 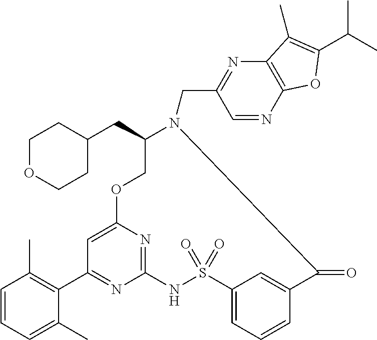 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCOCC1)CO2] |
| I-32 | 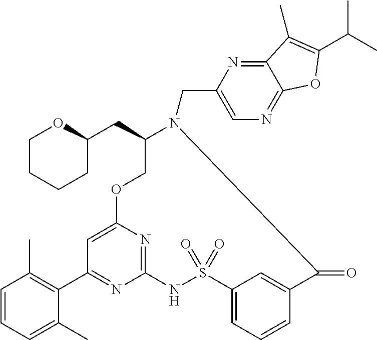 [SMILES: Cc1cccc(C)c1-c1cc2nc(n1)NS(=O)(=O)c1cccc(c1)C(=O)N(Cc1cnc3oc(C(C)C)c(C)c3n1)[C@H](C[C@H]1CCCCO1)CO2] |
| I-33 | 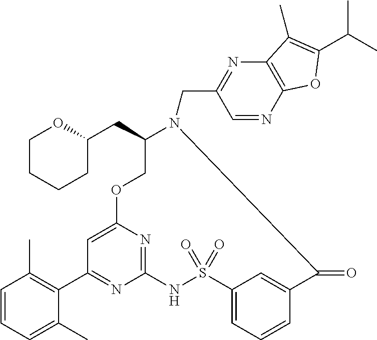 [SMILES: Cc1cccc(C)c1-c1cc2nc(n1)NS(=O)(=O)c1cccc(c1)C(=O)N(Cc1cnc3oc(C(C)C)c(C)c3n1)[C@H](C[C@@H]1CCCCO1)CO2] |
| I-34 | 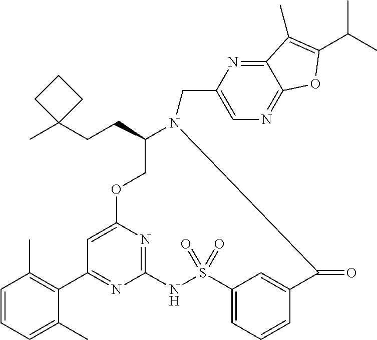 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC1(C)CCC1)CO2] |
| I-35 | 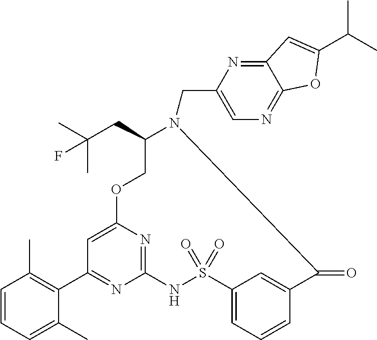 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)F)CO2] |
| I-36 | 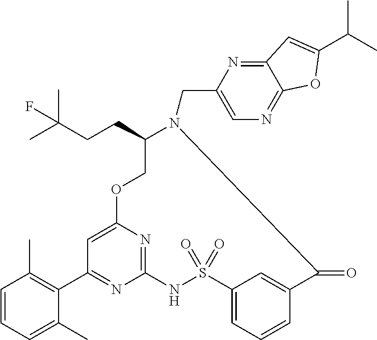 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)(C)F)CO2] |
| I-37 | 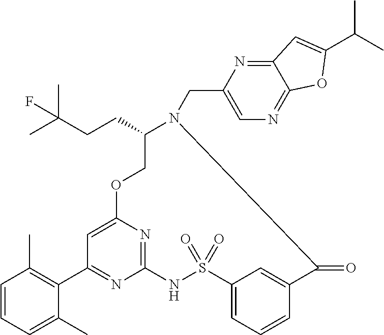 [SMILES: Cc1cccc(C)c1-c1cc2nc(n1)NS(=O)(=O)c1cccc(c1)C(=O)N(Cc1cnc3oc(C(C)C)cc3n1)[C@@H](CCC(C)(C)F)CO2] |
| I-38 | 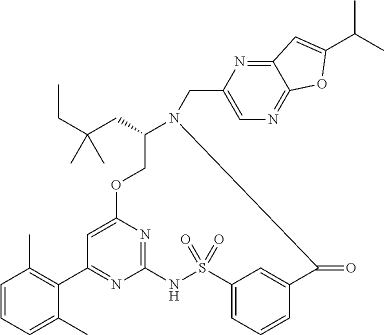 [SMILES: CCC(C)(C)C[C@H]1COc2cc(-c3c(C)cccc3C)nc(n2)NS(=O)(=O)c2cccc(c2)C(=O)N1Cc1cnc2oc(C(C)C)cc2n1] |
| I-39 | 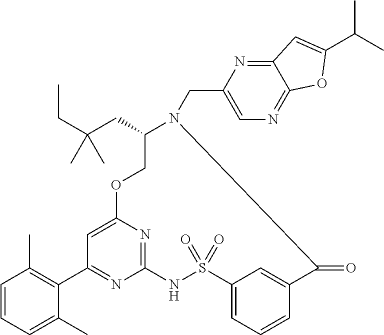 [SMILES: CCC(C)(C)C[C@H]1COc2cc(-c3c(C)cccc3C)nc(n2)NS(=O)(=O)c2cccc(c2)C(=O)N1Cc1cnc2oc(C(C)C)cc2n1] |
| I-40 | 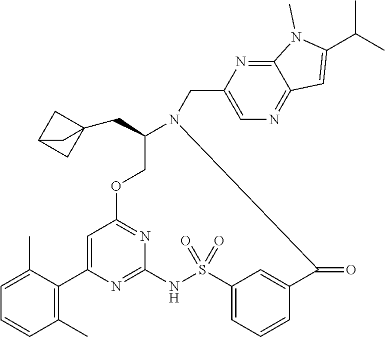 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC13CC(C1)C3)CO2] |
| I-41 | 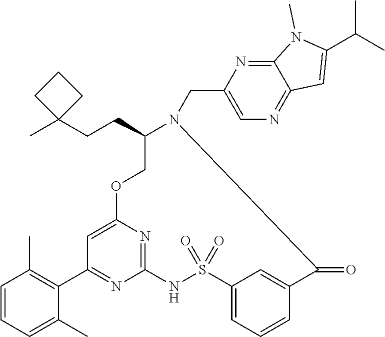 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1(C)CCC1)CO2] |
| I-42 | 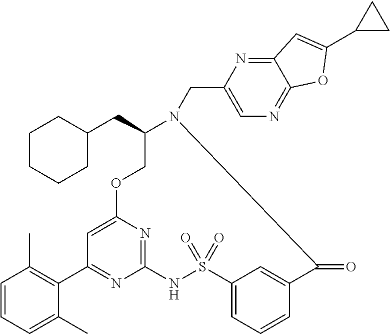 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCCC1)CO2] |
| I-43 | 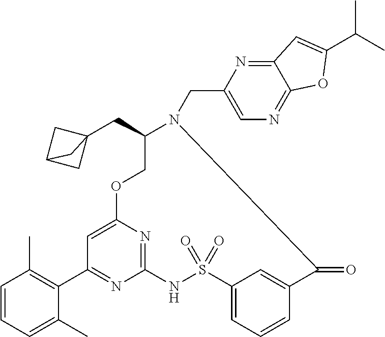 [SMILES: Cc1cccc(C)c1-c1cc2nc(n1)NS(=O)(=O)c1cccc(c1)C(=O)N(Cc1cnc3oc(C(C)C)cc3n1)[C@H](CC13CC(C1)C3)CO2] |
| I-44 | 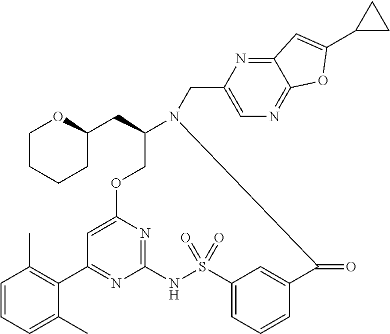 [SMILES: Cc1cccc(C)c1-c1cc2nc(n1)NS(=O)(=O)c1cccc(c1)C(=O)N(Cc1cnc3oc(C4CC4)cc3n1)[C@H](C[C@H]1CCCCO1)CO2] |
| I-45 | 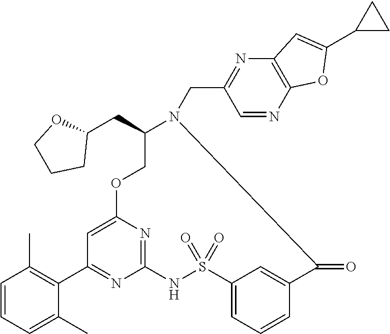 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCO1)CO2] |
| I-46 | 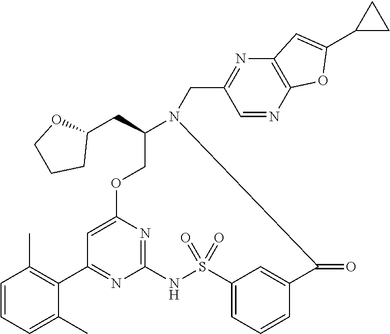 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCO1)CO2] |
| I-47 | 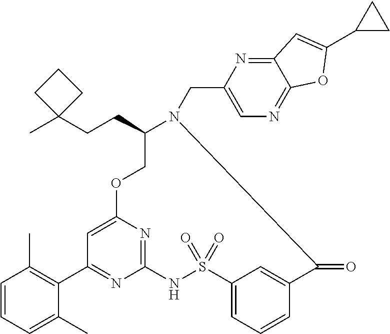 [SMILES: Cc1cccc(C)c1-c1cc2nc(n1)NS(=O)(=O)c1cccc(c1)C(=O)N(Cc1cnc3oc(C4CC4)cc3n1)[C@H](CCC1(C)CCC1)CO2] |
| I-48 | 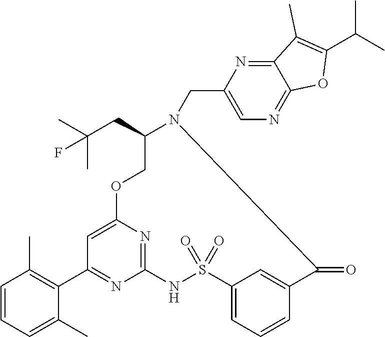 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)F)CO2] |
| I-49 | 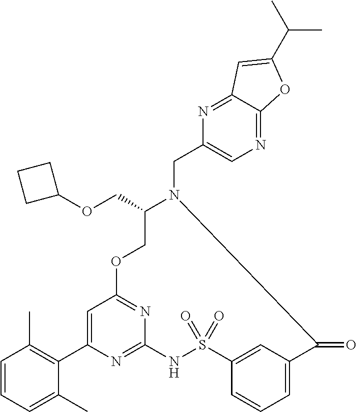 [SMILES: Cc1cccc(C)c1-c1cc2nc(n1)NS(=O)(=O)c1cccc(c1)C(=O)N(Cc1cnc3oc(C(C)C)cc3n1)[C@@H](COC1CCC1)CO2] |
| I-50 | 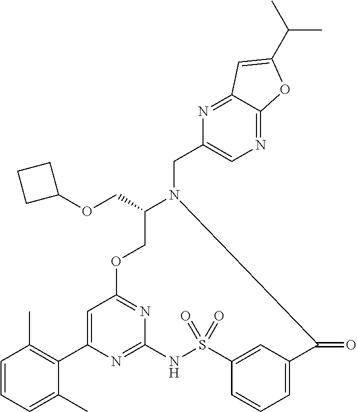 [SMILES: Cc1cccc(C)c1-c1cc2nc(n1)NS(=O)(=O)c1cccc(c1)C(=O)N(Cc1cnc3oc(C(C)C)cc3n1)[C@@H](COC1CCC1)CO2] |
| I-51 | 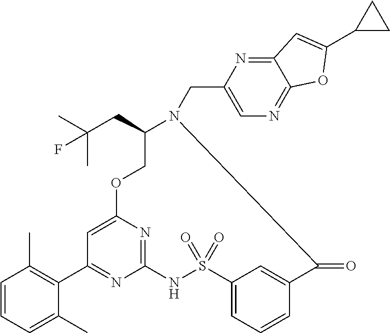 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)F)CO2] |
| I-52 | 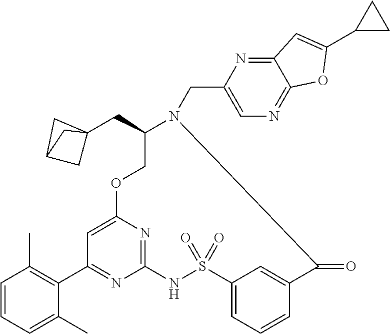 [SMILES: Cc1cccc(C)c1-c1cc2nc(n1)NS(=O)(=O)c1cccc(c1)C(=O)N(Cc1cnc3oc(C4CC4)cc3n1)[C@H](CC13CC(C1)C3)CO2] |
| I-53 | 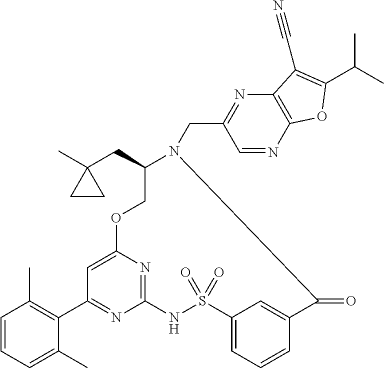 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC1(C)CC1)CO2] |
| I-54 | 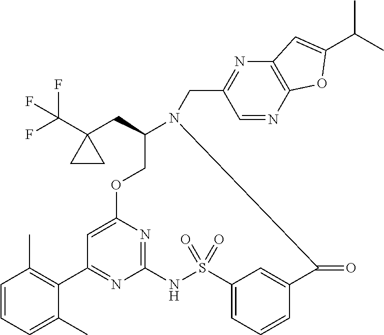 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C(F)(F)F)CC1)CO2] |
| I-55 | 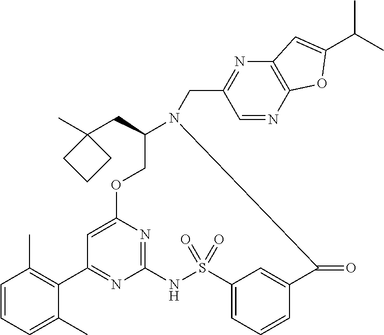 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C)CCC1)CO2] |
| I-56 | 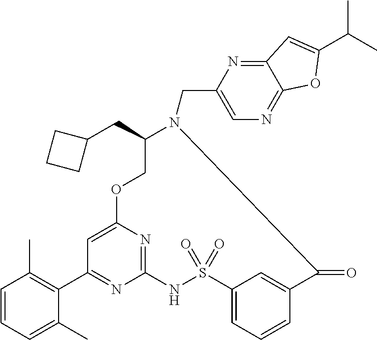 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCC1)CO2] |
| I-57 | 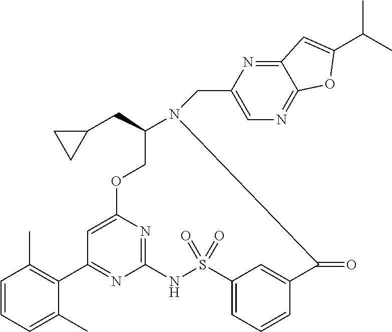 [SMILES: Cc1cccc(C)c1-c1cc2nc(n1)NS(=O)(=O)c1cccc(c1)C(=O)N(Cc1cnc3oc(C(C)C)cc3n1)[C@H](CC1CC1)CO2] |
| I-58 | 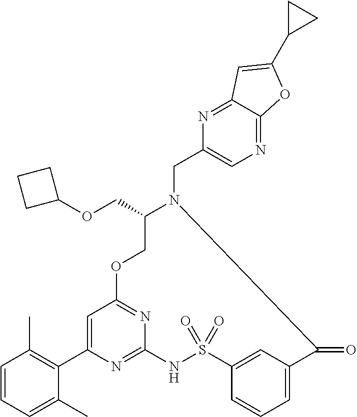 [SMILES: Cc1cccc(C)c1-c1cc2nc(n1)NS(=O)(=O)c1cccc(c1)C(=O)N(Cc1cnc3oc(C4CC4)cc3n1)[C@@H](COC1CCC1)CO2] |
| I-59 | 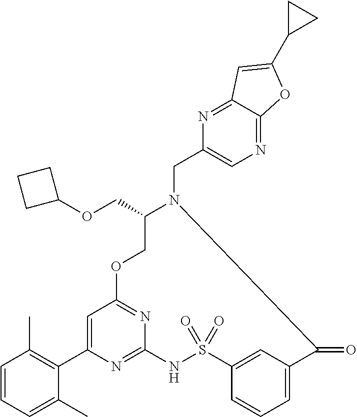 [SMILES: Cc1cccc(C)c1-c1cc2nc(n1)NS(=O)(=O)c1cccc(c1)C(=O)N(Cc1cnc3oc(C4CC4)cc3n1)[C@@H](COC1CCC1)CO2] |
| I-60 | 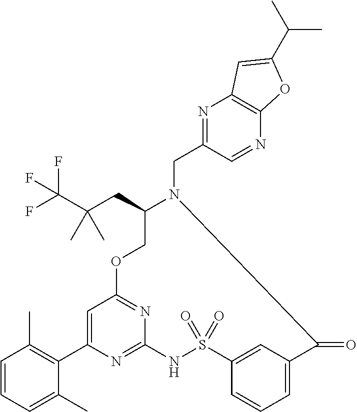 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C(F)(F)F)CO2] |
| I-61 | 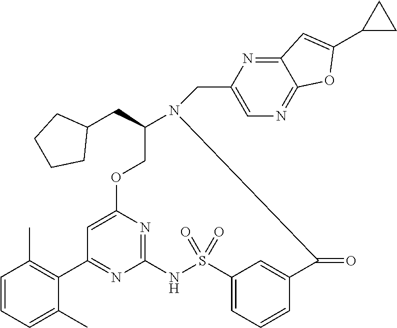 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCC1)CO2] |
| I-62 | 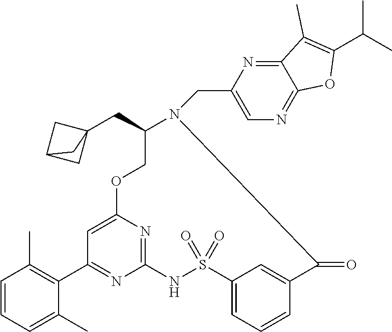 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC13CC(C1)C3)CO2] |
| I-63 | 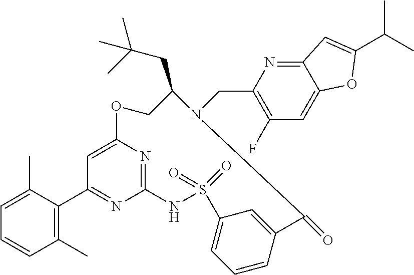 [SMILES: Cc1cccc(C)c1-c1cc2nc(n1)NS(=O)(=O)c1cccc(c1)C(=O)N(Cc1nc3cc(C(C)C)oc3cc1F)[C@H](CC(C)(C)C)CO2] |
| I-64 | 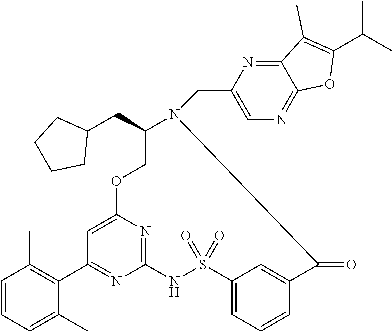 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCC1)CO2] |
| I-65 | 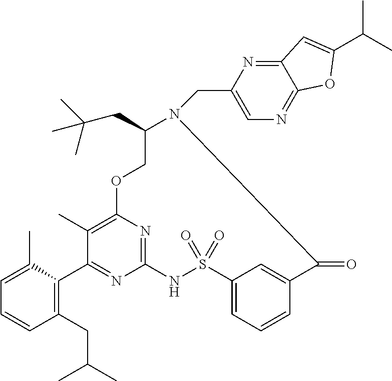 |
| I-66 | 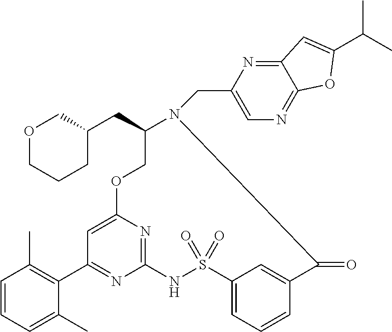 [SMILES: Cc1cccc(C)c1-c1cc2nc(n1)NS(=O)(=O)c1cccc(c1)C(=O)N(Cc1cnc3oc(C(C)C)cc3n1)[C@H](C[C@@H]1CCCOC1)CO2] |
| I-67 | 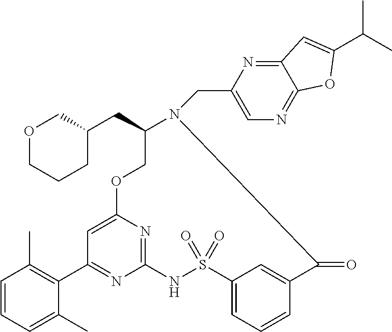 [SMILES: Cc1cccc(C)c1-c1cc2nc(n1)NS(=O)(=O)c1cccc(c1)C(=O)N(Cc1cnc3oc(C(C)C)cc3n1)[C@H](C[C@@H]1CCCOC1)CO2] |
| I-68 | 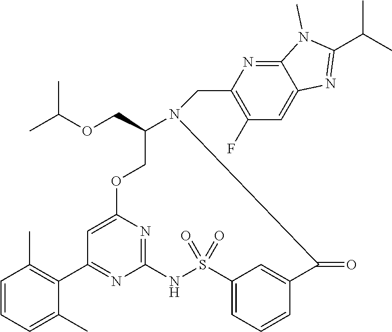 [SMILES: Cc1cccc(C)c1-c1cc2nc(n1)NS(=O)(=O)c1cccc(c1)C(=O)N(Cc1nc3c(cc1F)nc(C(C)C)n3C)[C@H](COC(C)C)CO2] |
| I-69 | 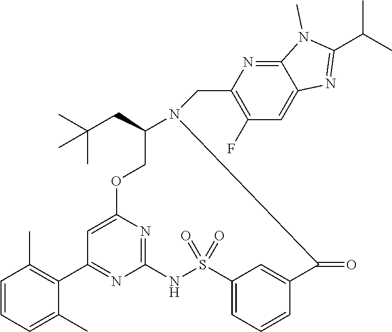 [SMILES: Cc1cccc(C)c1-c1cc2nc(n1)NS(=O)(=O)c1cccc(c1)C(=O)N(Cc1nc3c(cc1F)nc(C(C)C)n3C)[C@H](CC(C)(C)C)CO2] |
| I-70 | 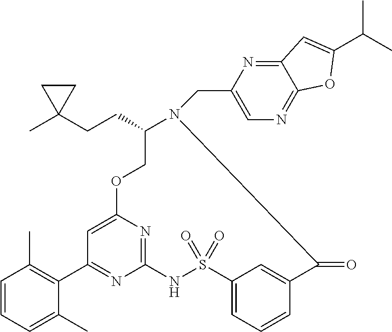 [SMILES: Cc1cccc(C)c1-c1cc2nc(n1)NS(=O)(=O)c1cccc(c1)C(=O)N(Cc1cnc3oc(C(C)C)cc3n1)[C@@H](CCC1(C)CC1)CO2] |
| I-71 | 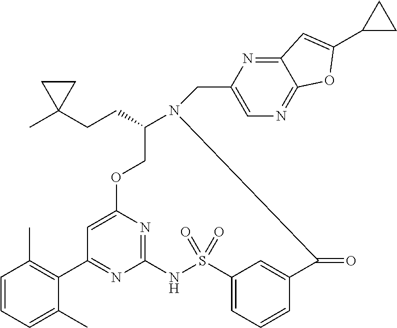 [SMILES: Cc1cccc(C)c1-c1cc2nc(n1)NS(=O)(=O)c1cccc(c1)C(=O)N(Cc1cnc3oc(C4CC4)cc3n1)[C@@H](CCC1(C)CC1)CO2] |
| I-72 | 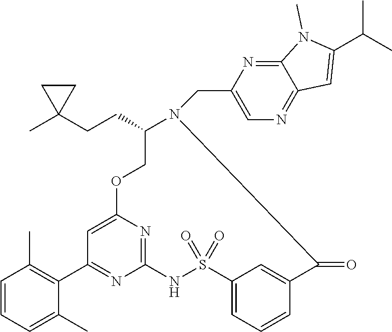 [SMILES: Cc1cccc(C)c1-c1cc2nc(n1)NS(=O)(=O)c1cccc(c1)C(=O)N(Cc1cnc3cc(C(C)C)n(C)c3n1)[C@@H](CCC1(C)CC1)CO2] |
| I-73 | 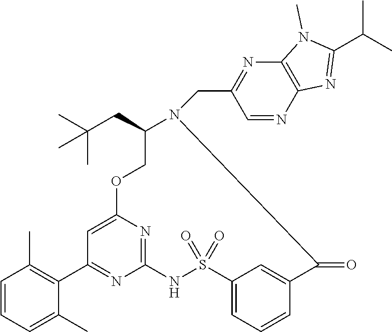 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CO2] |
| I-74 | 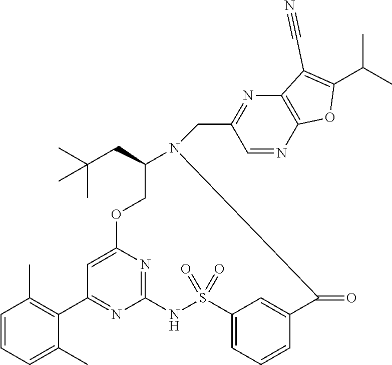 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC(C)(C)C)CO2] |
| I-75 | 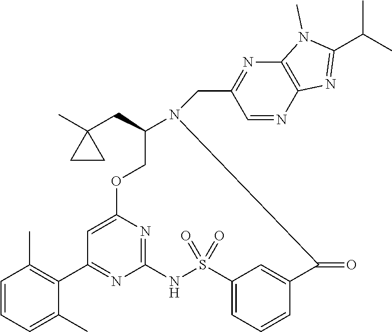 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC1(C)CC1)CO2] |
| I-76 | 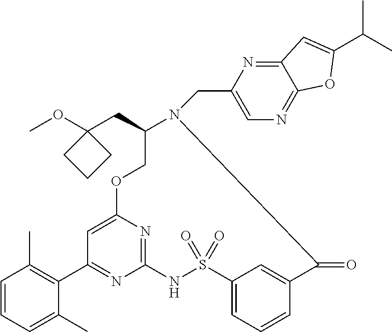 [SMILES: COC1(C[C@@H]2COc3cc(-c4c(C)cccc4C)nc(n3)NS(=O)(=O)c3cccc(c3)C(=O)N2Cc2cnc3oc(C(C)C)cc3n2)CCC1] |
| I-77 | 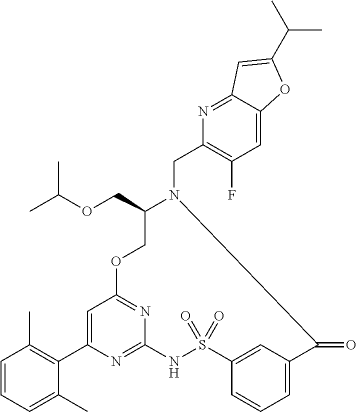 [SMILES: Cc1cccc(C)c1-c1cc2nc(n1)NS(=O)(=O)c1cccc(c1)C(=O)N(Cc1nc3cc(C(C)C)oc3cc1F)[C@H](COC(C)C)CO2] |
| I-78 | 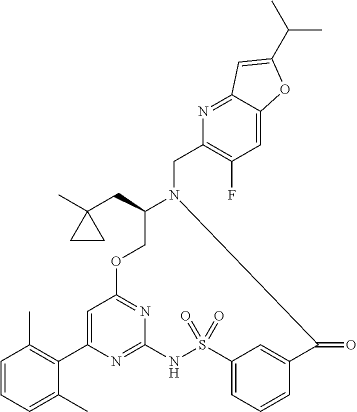 [SMILES: Cc1cccc(C)c1-c1cc2nc(n1)NS(=O)(=O)c1cccc(c1)C(=O)N(Cc1nc3cc(C(C)C)oc3cc1F)[C@H](CC1(C)CC1)CO2] |
| I-79 | 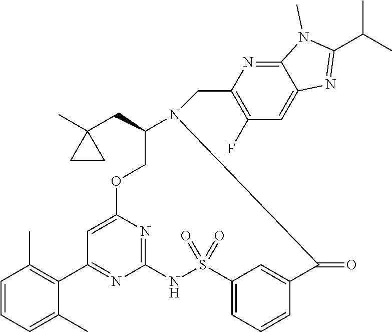 [SMILES: Cc1cccc(C)c1-c1cc2nc(n1)NS(=O)(=O)c1cccc(c1)C(=O)N(Cc1nc3c(cc1F)nc(C(C)C)n3C)[C@H](CC1(C)CC1)CO2] |
| I-80 | 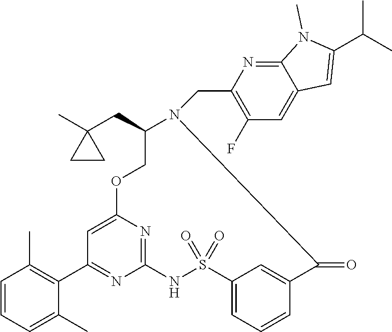 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC1(C)CC1)CO2] |
| I-81 | 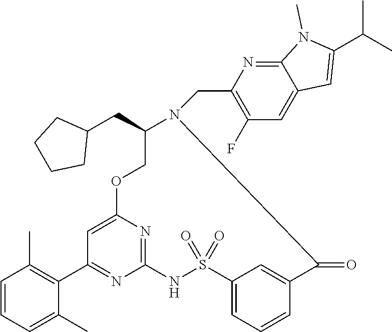 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC1CCCC1)CO2] |
| I-82 | 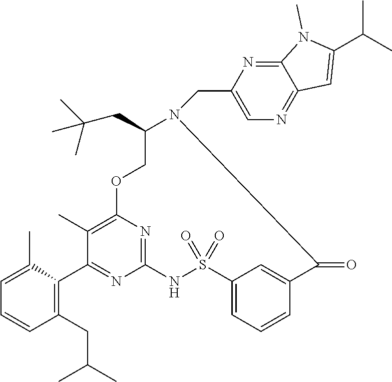 |
| I-83 | 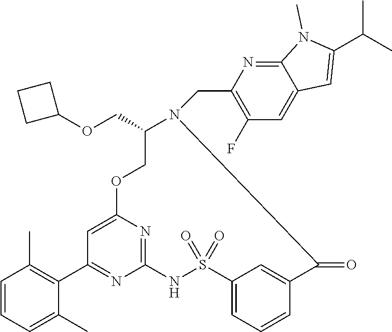 [SMILES: Cc1cccc(C)c1-c1cc2nc(n1)NS(=O)(=O)c1cccc(c1)C(=O)N(Cc1nc3c(cc1F)cc(C(C)C)n3C)[C@@H](COC1CCC1)CO2] |
| I-84 | 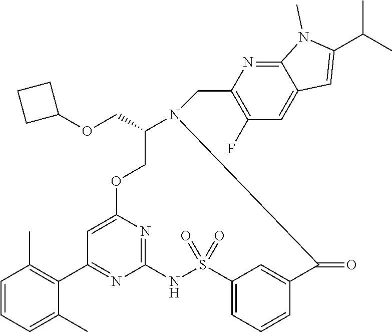 [SMILES: Cc1cccc(C)c1-c1cc2nc(n1)NS(=O)(=O)c1cccc(c1)C(=O)N(Cc1nc3c(cc1F)cc(C(C)C)n3C)[C@@H](COC1CCC1)CO2] |
| I-85 | 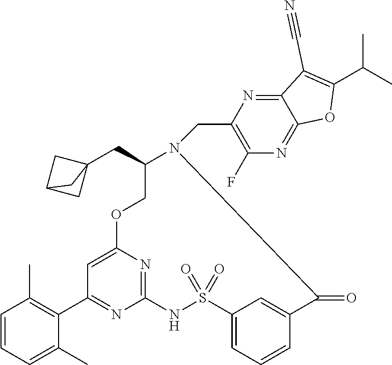 [SMILES: Cc1cccc(C)c1-c1cc2nc(n1)NS(=O)(=O)c1cccc(c1)C(=O)N(Cc1nc3c(C#N)c(C(C)C)oc3nc1F)[C@H](CC13CC(C1)C3)CO2] |
| I-86 | 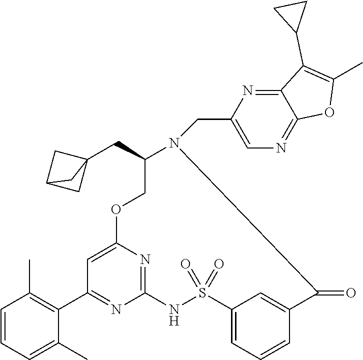 [SMILES: Cc1cccc(C)c1-c1cc2nc(n1)NS(=O)(=O)c1cccc(c1)C(=O)N(Cc1cnc3oc(C)c(C4CC4)c3n1)[C@H](CC13CC(C1)C3)CO2] |
| I-87 | 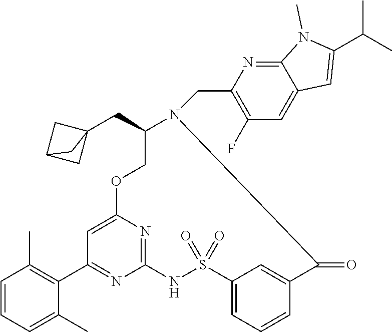 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC13CC(C1)C3)CO2] |
| I-88 | 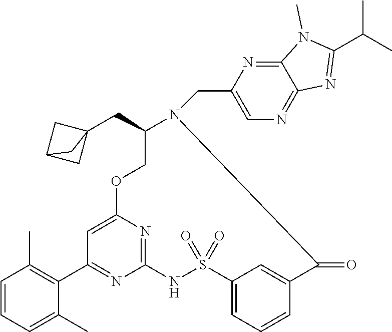 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC13CC(C1)C3)CO2] |
| I-89 | 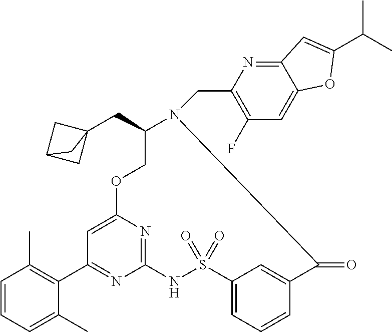 [SMILES: Cc1cccc(C)c1-c1cc2nc(n1)NS(=O)(=O)c1cccc(c1)C(=O)N(Cc1nc3cc(C(C)C)oc3cc1F)[C@H](CC13CC(C1)C3)CO2] |
| I-90 | 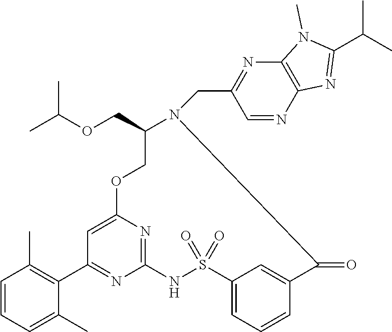 [SMILES: Cc1cccc(C)c1-c1cc2nc(n1)NS(=O)(=O)c1cccc(c1)C(=O)N(Cc1cnc3nc(C(C)C)n(C)c3n1)[C@H](COC(C)C)CO2] |
| I-91 | 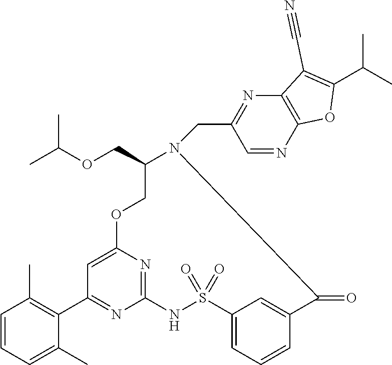 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](COC(C)C)CO2] |
| I-92 | 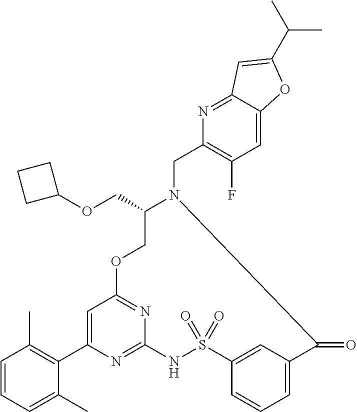 [SMILES: Cc1cccc(C)c1-c1cc2nc(n1)NS(=O)(=O)c1cccc(c1)C(=O)N(Cc1nc3cc(C(C)C)oc3cc1F)[C@@H](COC1CCC1)CO2] |
| I-93 | 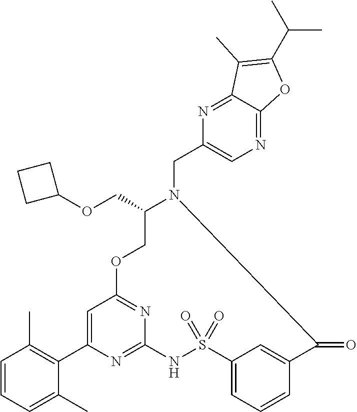 [SMILES: Cc1cccc(C)c1-c1cc2nc(n1)NS(=O)(=O)c1cccc(c1)C(=O)N(Cc1cnc3oc(C(C)C)c(C)c3n1)[C@@H](COC1CCC1)CO2] |
| I-94 | 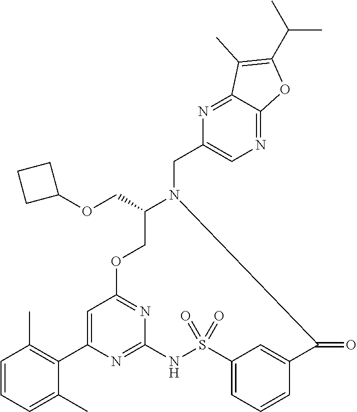 [SMILES: Cc1cccc(C)c1-c1cc2nc(n1)NS(=O)(=O)c1cccc(c1)C(=O)N(Cc1cnc3oc(C(C)C)c(C)c3n1)[C@@H](COC1CCC1)CO2] |
| I-95 | 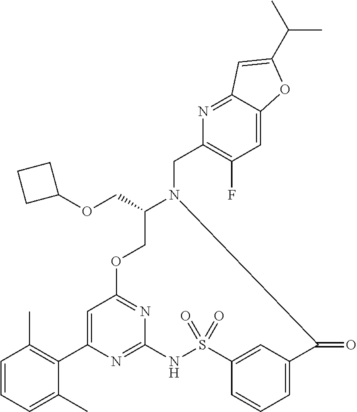 [SMILES: Cc1cccc(C)c1-c1cc2nc(n1)NS(=O)(=O)c1cccc(c1)C(=O)N(Cc1nc3cc(C(C)C)oc3cc1F)[C@@H](COC1CCC1)CO2] |
| I-96 | 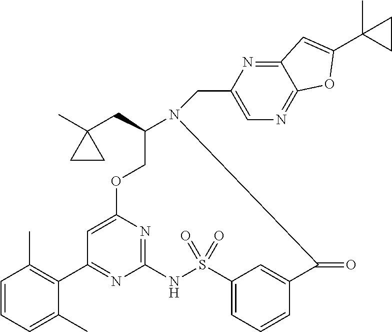 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC1(C)CC1)CO2] |
| I-97 | 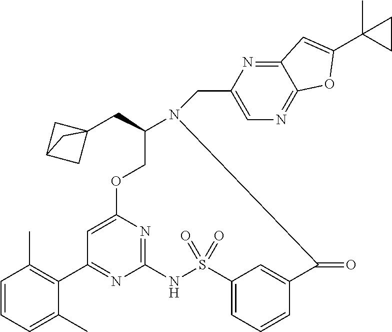 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC13CC(C1)C3)CO2] |
| I-98 | 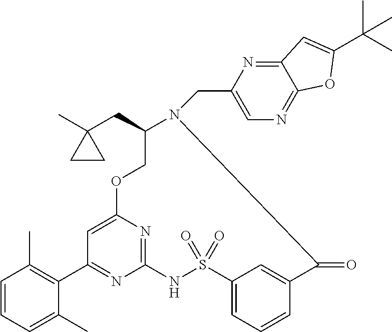 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1(C)CC1)CO2] |
| I-99 | 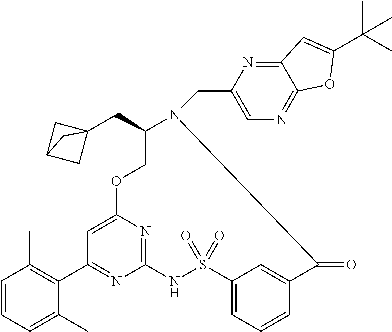 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC13CC(C1)C3)CO2] |
| I-100 | 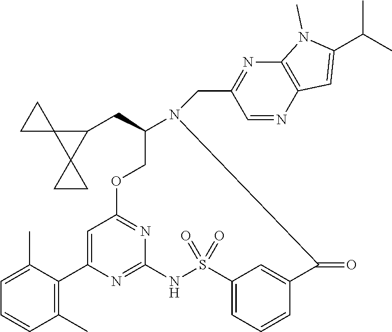 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1C3(CC3)C13CC3)CO2] |
| I-101 | 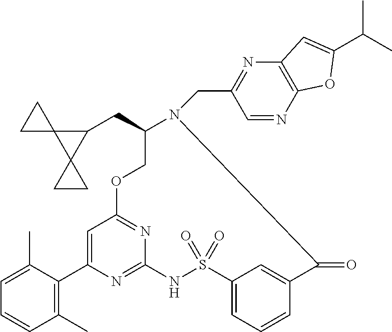 [SMILES: Cc1cccc(C)c1-c1cc2nc(n1)NS(=O)(=O)c1cccc(c1)C(=O)N(Cc1cnc3oc(C(C)C)cc3n1)[C@H](CC1C3(CC3)C13CC3)CO2] |
| I-102 | 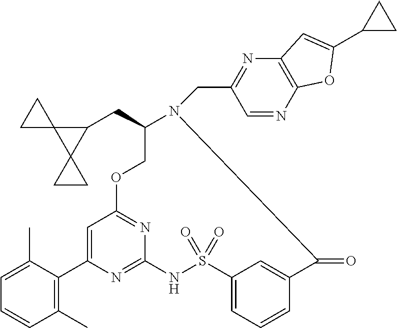 [SMILES: Cc1cccc(C)c1-c1cc2nc(n1)NS(=O)(=O)c1cccc(c1)C(=O)N(Cc1cnc3oc(C4CC4)cc3n1)[C@H](CC1C3(CC3)C13CC3)CO2] |
| I-103 | 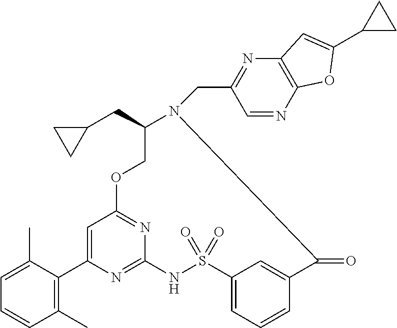 [SMILES: Cc1cccc(C)c1-c1cc2nc(n1)NS(=O)(=O)c1cccc(c1)C(=O)N(Cc1cnc3oc(C4CC4)cc3n1)[C@H](CC1CC1)CO2] |
| I-104 | 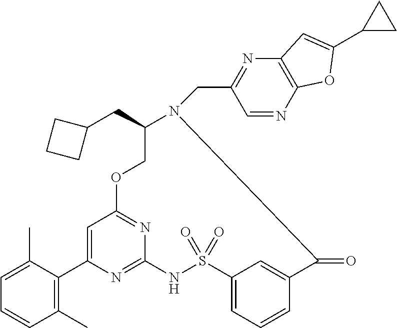 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCC1)CO2] |
| I-105 | 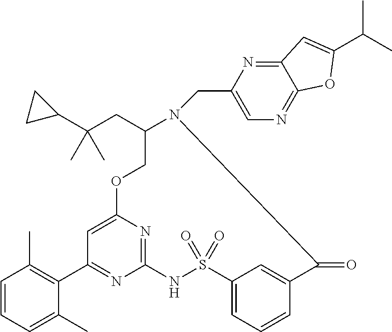 [SMILES: Cc1cccc(C)c1-c1cc2nc(n1)NS(=O)(=O)c1cccc(c1)C(=O)N(Cc1cnc3oc(C(C)C)cc3n1)C(CC(C)(C)C1CC1)CO2] |
| I-106 | 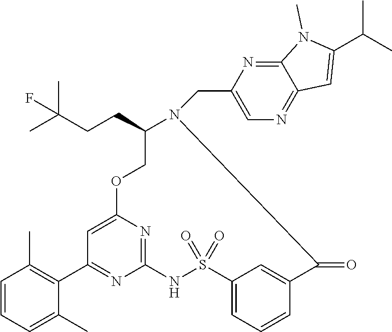 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)F)CO2] |
| I-107 | 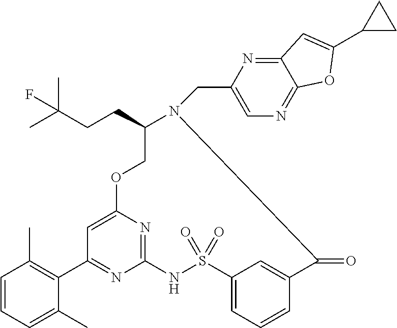 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)F)CO2] |
| I-108 | 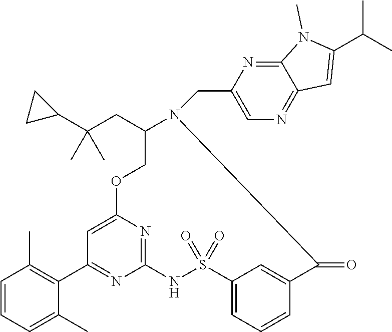 [SMILES: Cc1cccc(C)c1-c1cc2nc(n1)NS(=O)(=O)c1cccc(c1)C(=O)N(Cc1cnc3cc(C(C)C)n(C)c3n1)C(CC(C)(C)C1CC1)CO2] |
| I-109 | 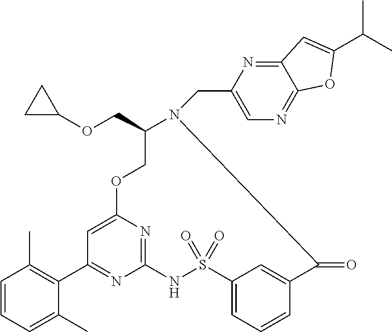 [SMILES: Cc1cccc(C)c1-c1cc2nc(n1)NS(=O)(=O)c1cccc(c1)C(=O)N(Cc1cnc3oc(C(C)C)cc3n1)[C@H](COC1CC1)CO2] |
| I-110 | 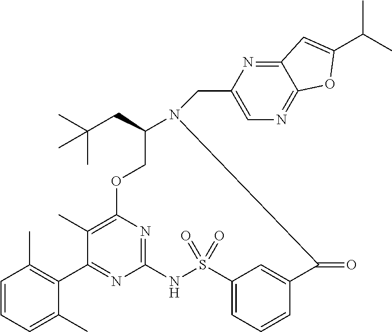 [SMILES: Cc1cccc(C)c1-c1nc2nc(c1C)OC[C@@H](CC(C)(C)C)N(Cc1cnc3oc(C(C)C)cc3n1)C(=O)c1cccc(c1)S(=O)(=O)N2] |
| I-111 | 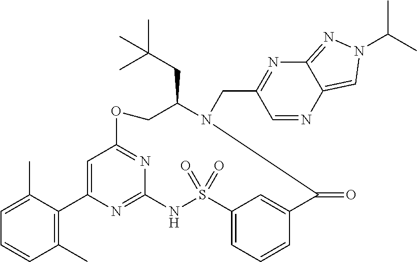 [SMILES: Cc1cccc(C)c1-c1cc2nc(n1)NS(=O)(=O)c1cccc(c1)C(=O)N(Cc1cnc3cn(C(C)C)nc3n1)[C@H](CC(C)(C)C)CO2] |
| I-112 | 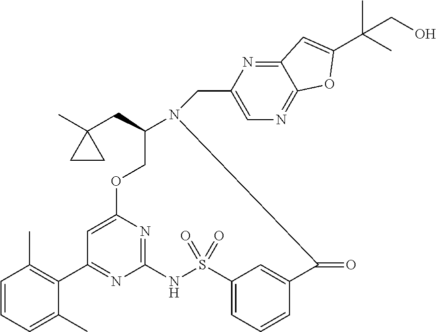 [SMILES: Cc1cccc(C)c1-c1cc2nc(n1)NS(=O)(=O)c1cccc(c1)C(=O)N(Cc1cnc3oc(C(C)(C)CO)cc3n1)[C@H](CC1(C)CC1)CO2] |
| I-113 | 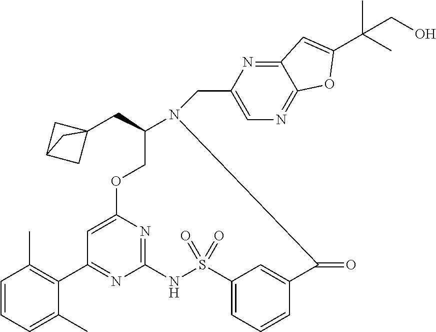 [SMILES: Cc1cccc(C)c1-c1cc2nc(n1)NS(=O)(=O)c1cccc(c1)C(=O)N(Cc1cnc3oc(C(C)(C)CO)cc3n1)[C@H](CC13CC(C1)C3)CO2] |
| I-114 | 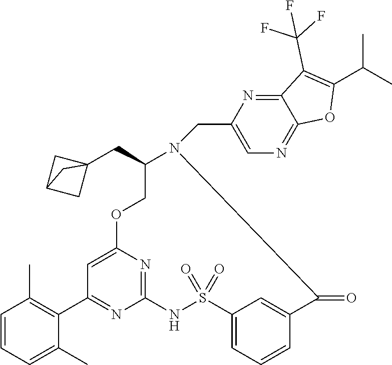 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@H](CC13CC(C1)C3)CO2] |
| I-115 | 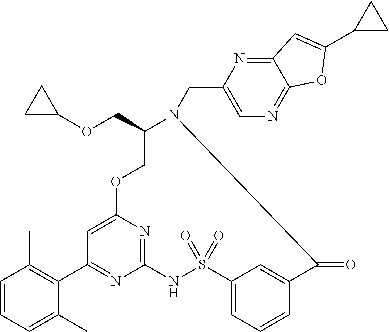 [SMILES: Cc1cccc(C)c1-c1cc2nc(n1)NS(=O)(=O)c1cccc(c1)C(=O)N(Cc1cnc3oc(C4CC4)cc3n1)[C@H](COC1CC1)CO2] |
| I-116 | 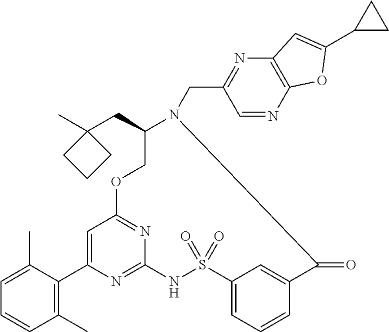 [SMILES: Cc1cccc(C)c1-c1cc2nc(n1)NS(=O)(=O)c1cccc(c1)C(=O)N(Cc1cnc3oc(C4CC4)cc3n1)[C@H](CC1(C)CCC1)CO2] |
| I-117 | 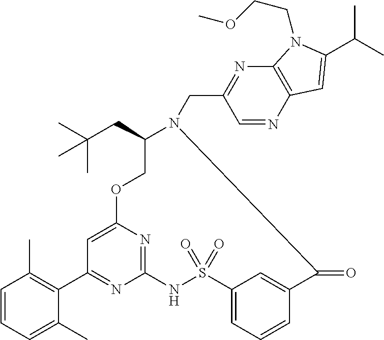 [SMILES: COCCn1c(C(C)C)cc2ncc(CN3C(=O)c4cccc(c4)S(=O)(=O)Nc4nc(cc(-c5c(C)cccc5C)n4)OC[C@H]3CC(C)(C)C)nc21] |
| I-118 | 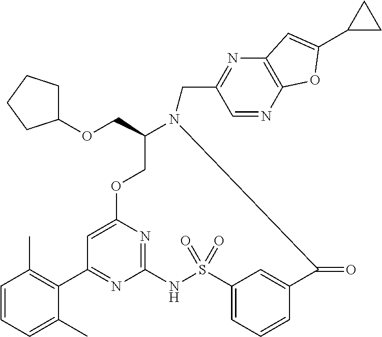 [SMILES: Cc1cccc(C)c1-c1cc2nc(n1)NS(=O)(=O)c1cccc(c1)C(=O)N(Cc1cnc3oc(C4CC4)cc3n1)[C@H](COC1CCCC1)CO2] |
| I-119 | 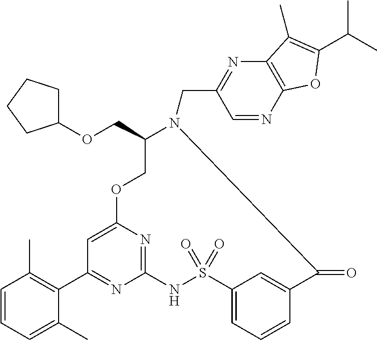 [SMILES: Cc1cccc(C)c1-c1cc2nc(n1)NS(=O)(=O)c1cccc(c1)C(=O)N(Cc1cnc3oc(C(C)C)c(C)c3n1)[C@H](COC1CCCC1)CO2] |
| I-120 | 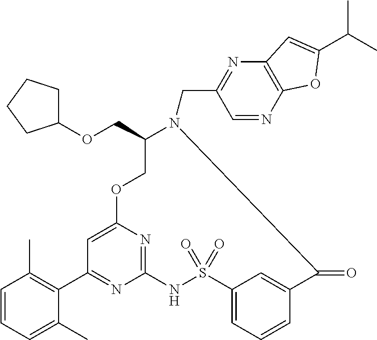 [SMILES: Cc1cccc(C)c1-c1cc2nc(n1)NS(=O)(=O)c1cccc(c1)C(=O)N(Cc1cnc3oc(C(C)C)cc3n1)[C@H](COC1CCCC1)CO2] |
| I-121 | 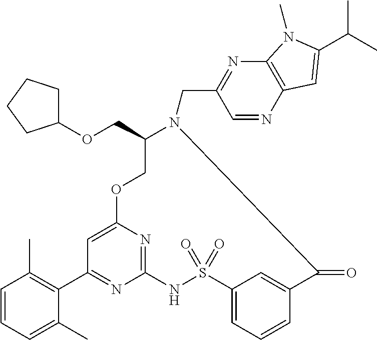 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC1CCCC1)CO2] |
| I-122 | 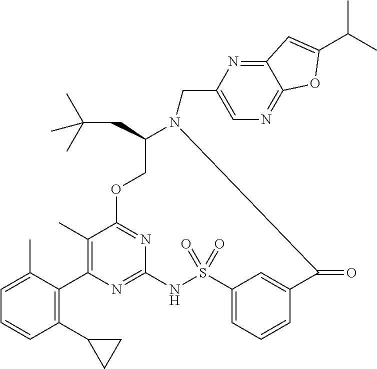 [SMILES: Cc1cccc(C2CC2)c1-c1nc2nc(c1C)OC[C@@H](CC(C)(C)C)N(Cc1cnc3oc(C(C)C)cc3n1)C(=O)c1cccc(c1)S(=O)(=O)N2] |
| I-123 | 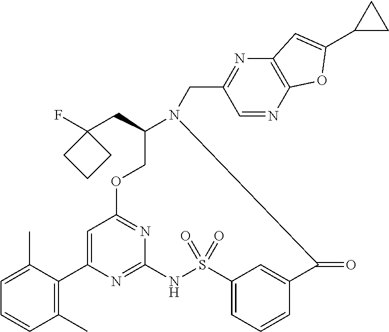 [SMILES: Cc1cccc(C)c1-c1cc2nc(n1)NS(=O)(=O)c1cccc(c1)C(=O)N(Cc1cnc3oc(C4CC4)cc3n1)[C@H](CC1(F)CCC1)CO2] |
| I-124 | 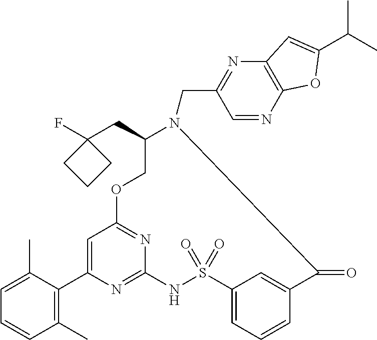 [SMILES: Cc1cccc(C)c1-c1cc2nc(n1)NS(=O)(=O)c1cccc(c1)C(=O)N(Cc1cnc3oc(C(C)C)cc3n1)[C@H](CC1(F)CCC1)CO2] |
| I-125 | 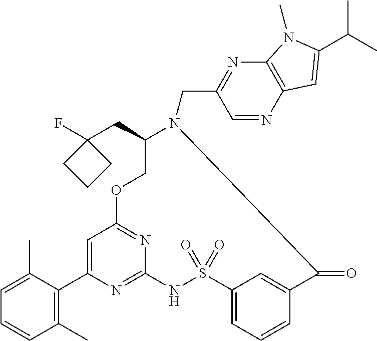 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(F)CCC1)CO2] |
| I-126 | 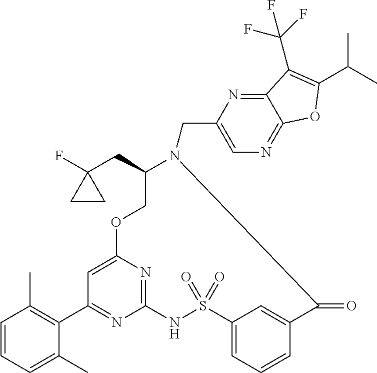 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@H](CC1(F)CC1)CO2] |
| I-127 | 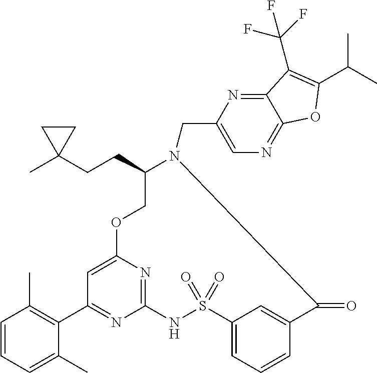 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@H](CCC1(C)CC1)CO2] |
| I-128 | 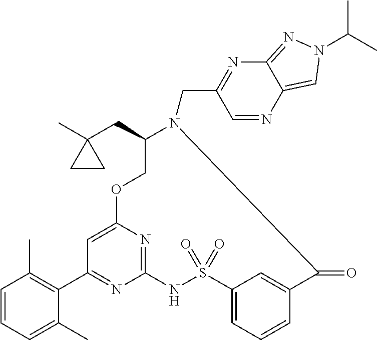 [SMILES: Cc1cccc(C)c1-c1cc2nc(n1)NS(=O)(=O)c1cccc(c1)C(=O)N(Cc1cnc3cn(C(C)C)nc3n1)[C@H](CC1(C)CC1)CO2] |
| I-129 | 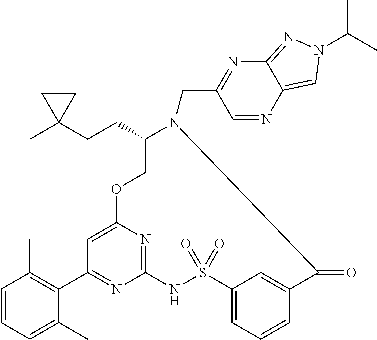 [SMILES: Cc1cccc(C)c1-c1cc2nc(n1)NS(=O)(=O)c1cccc(c1)C(=O)N(Cc1cnc3cn(C(C)C)nc3n1)[C@@H](CCC1(C)CC1)CO2] |
| I-130 | 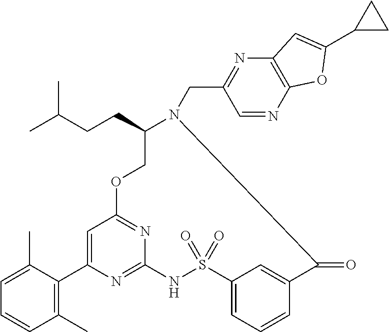 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)C)CO2] |
| I-131 | 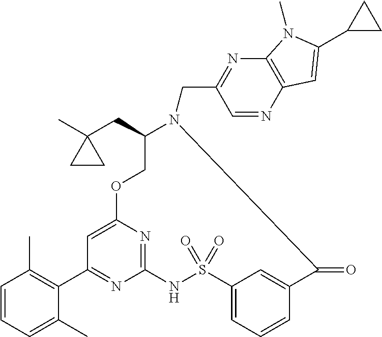 [SMILES: Cc1cccc(C)c1-c1cc2nc(n1)NS(=O)(=O)c1cccc(c1)C(=O)N(Cc1cnc3cc(C4CC4)n(C)c3n1)[C@H](CC1(C)CC1)CO2] |
| I-132 | 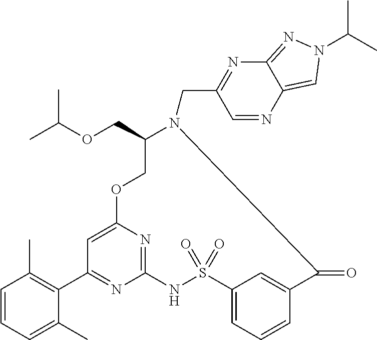 [SMILES: Cc1cccc(C)c1-c1cc2nc(n1)NS(=O)(=O)c1cccc(c1)C(=O)N(Cc1cnc3cn(C(C)C)nc3n1)[C@H](COC(C)C)CO2] |
| I-133 | 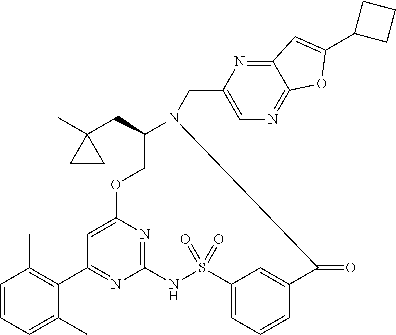 [SMILES: Cc1cccc(C)c1-c1cc2nc(n1)NS(=O)(=O)c1cccc(c1)C(=O)N(Cc1cnc3oc(C4CCC4)cc3n1)[C@H](CC1(C)CC1)CO2] |
| I-134 | 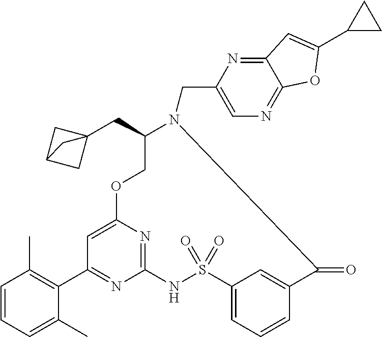 [SMILES: Cc1cccc(C)c1-c1cc2nc(n1)NS(=O)(=O)c1cccc(c1)C(=O)N(Cc1cnc3oc(C4CC4)cc3n1)[C@H](CC13CC(C1)C3)CO2] |
| I-135 | 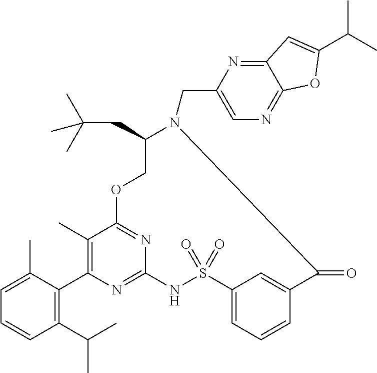 [SMILES: Cc1cccc(C(C)C)c1-c1nc2nc(c1C)OC[C@@H](CC(C)(C)C)N(Cc1cnc3oc(C(C)C)cc3n1)C(=O)c1cccc(c1)S(=O)(=O)N2] |
| I-136 | 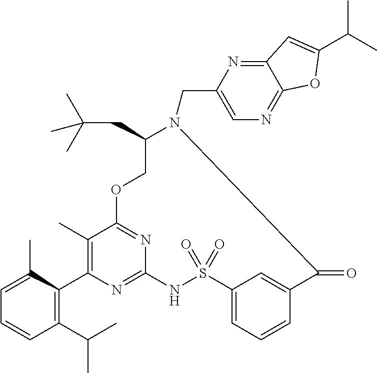 |
| I-137 | 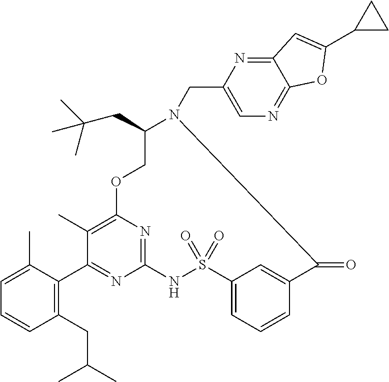 [SMILES: Cc1cccc(CC(C)C)c1-c1nc2nc(c1C)OC[C@@H](CC(C)(C)C)N(Cc1cnc3oc(C4CC4)cc3n1)C(=O)c1cccc(c1)S(=O)(=O)N2] |
| I-138 | 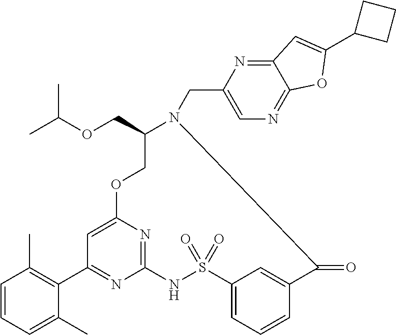 [SMILES: Cc1cccc(C)c1-c1cc2nc(n1)NS(=O)(=O)c1cccc(c1)C(=O)N(Cc1cnc3oc(C4CCC4)cc3n1)[C@H](COC(C)C)CO2] |
| I-139 | 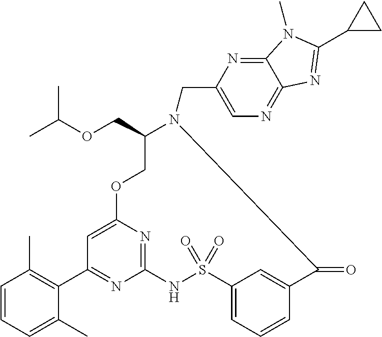 [SMILES: Cc1cccc(C)c1-c1cc2nc(n1)NS(=O)(=O)c1cccc(c1)C(=O)N(Cc1cnc3nc(C4CC4)n(C)c3n1)[C@H](COC(C)C)CO2] |
| I-140 | 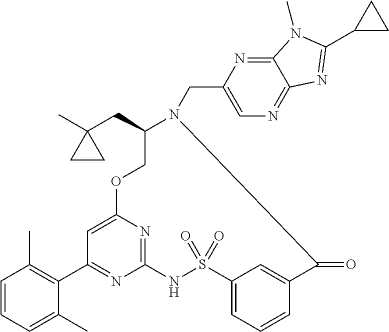 [SMILES: Cc1cccc(C)c1-c1cc2nc(n1)NS(=O)(=O)c1cccc(c1)C(=O)N(Cc1cnc3nc(C4CC4)n(C)c3n1)[C@H](CC1(C)CC1)CO2] |
| I-141 | 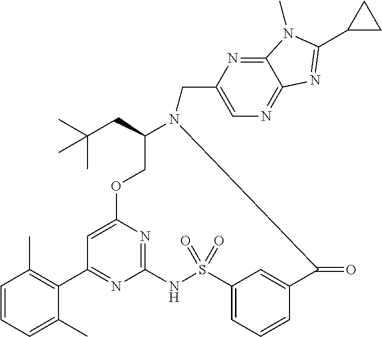 [SMILES: Cc1cccc(C)c1-c1cc2nc(n1)NS(=O)(=O)c1cccc(c1)C(=O)N(Cc1cnc3nc(C4CC4)n(C)c3n1)[C@H](CC(C)(C)C)CO2] |
| I-142 | 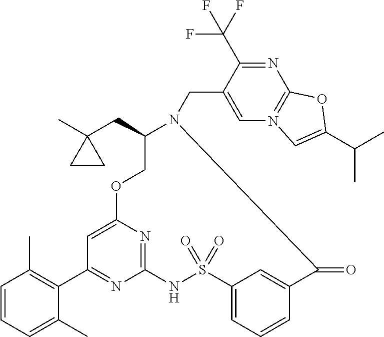 |
| I-143 | 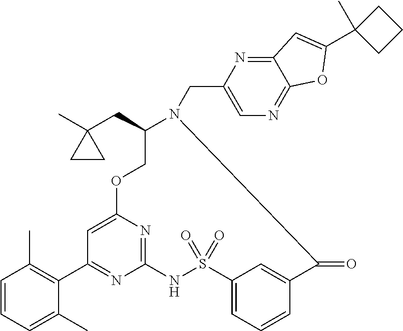 [SMILES: Cc1cccc(C)c1-c1cc2nc(n1)NS(=O)(=O)c1cccc(c1)C(=O)N(Cc1cnc3oc(C4(C)CCC4)cc3n1)[C@H](CC1(C)CC1)CO2] |
| I-144 | 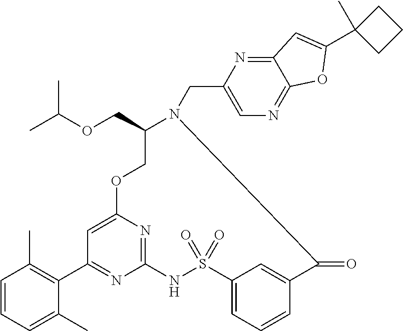 [SMILES: Cc1cccc(C)c1-c1cc2nc(n1)NS(=O)(=O)c1cccc(c1)C(=O)N(Cc1cnc3oc(C4(C)CCC4)cc3n1)[C@H](COC(C)C)CO2] |
| I-145 | 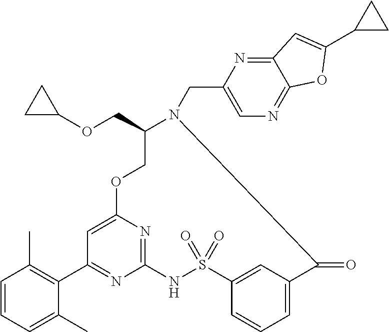 [SMILES: Cc1cccc(C)c1-c1cc2nc(n1)NS(=O)(=O)c1cccc(c1)C(=O)N(Cc1cnc3oc(C4CC4)cc3n1)[C@H](COC1CC1)CO2] |
| I-146 | 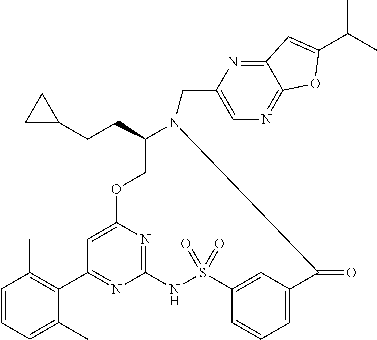 [SMILES: Cc1cccc(C)c1-c1cc2nc(n1)NS(=O)(=O)c1cccc(c1)C(=O)N(Cc1cnc3oc(C(C)C)cc3n1)[C@H](CCC1CC1)CO2] |
| I-147 | 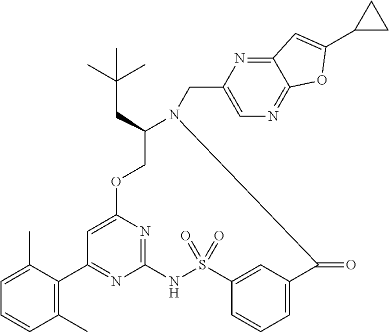 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)C)CO2] |
| I-148 | 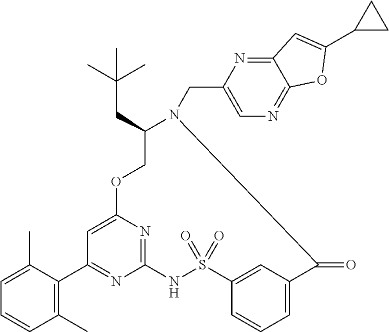 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)C)CO2] |
| I-149 | 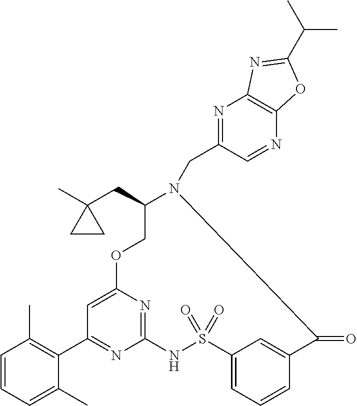 [SMILES: Cc1cccc(C)c1-c1cc2nc(n1)NS(=O)(=O)c1cccc(c1)C(=O)N(Cc1cnc3oc(C(C)C)nc3n1)[C@H](CC1(C)CC1)CO2] |
| I-150 | 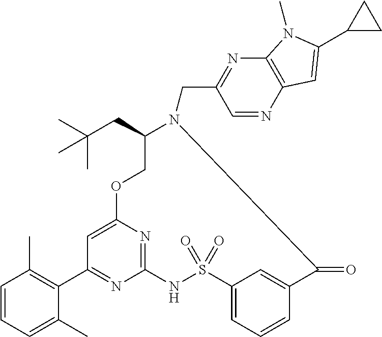 [SMILES: Cc1cccc(C)c1-c1cc2nc(n1)NS(=O)(=O)c1cccc(c1)C(=O)N(Cc1cnc3cc(C4CC4)n(C)c3n1)[C@H](CC(C)(C)C)CO2] |
| I-151 | 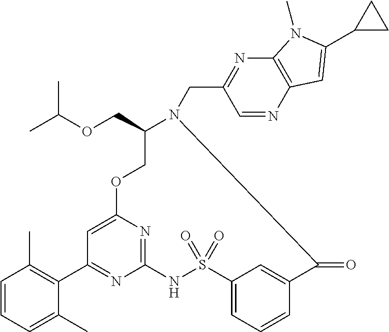 [SMILES: Cc1cccc(C)c1-c1cc2nc(n1)NS(=O)(=O)c1cccc(c1)C(=O)N(Cc1cnc3cc(C4CC4)n(C)c3n1)[C@H](COC(C)C)CO2] |
| I-152 | 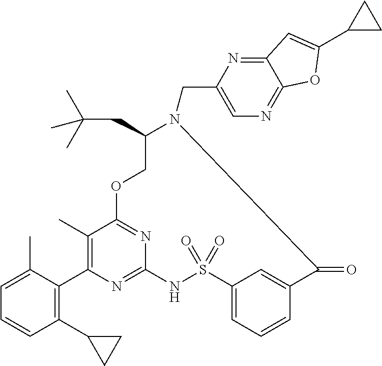 [SMILES: Cc1cccc(C2CC2)c1-c1nc2nc(c1C)OC[C@@H](CC(C)(C)C)N(Cc1cnc3oc(C4CC4)cc3n1)C(=O)c1cccc(c1)S(=O)(=O)N2] |
| I-153 | 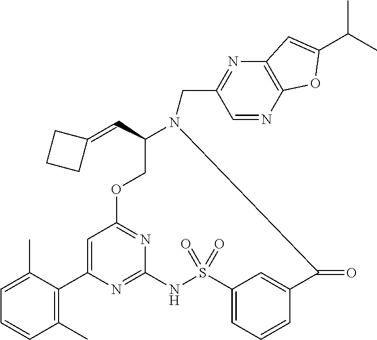 [SMILES: Cc1cccc(C)c1-c1cc2nc(n1)NS(=O)(=O)c1cccc(c1)C(=O)N(Cc1cnc3oc(C(C)C)cc3n1)[C@H](C=C1CCC1)CO2] |
| I-154 | 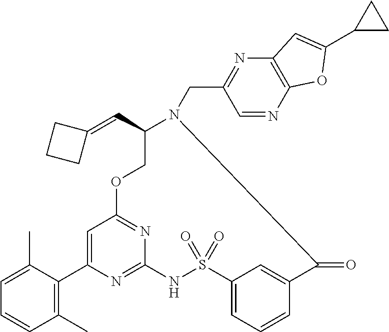 [SMILES: Cc1cccc(C)c1-c1cc2nc(n1)NS(=O)(=O)c1cccc(c1)C(=O)N(Cc1cnc3oc(C4CC4)cc3n1)[C@H](C=C1CCC1)CO2] |
| I-155 | 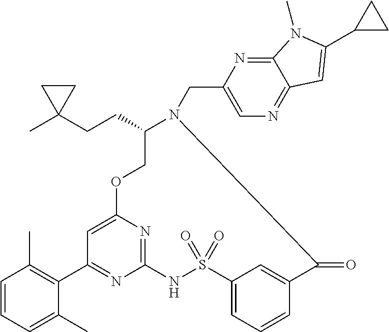 [SMILES: Cc1cccc(C)c1-c1cc2nc(n1)NS(=O)(=O)c1cccc(c1)C(=O)N(Cc1cnc3cc(C4CC4)n(C)c3n1)[C@@H](CCC1(C)CC1)CO2] |
| I-156 | 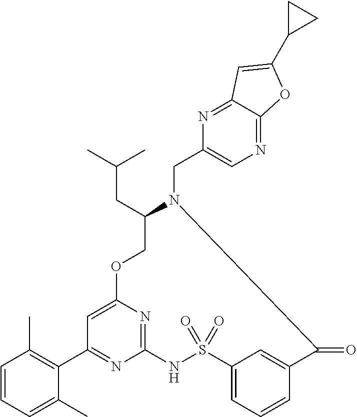 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)C)CO2] |
| I-157 | 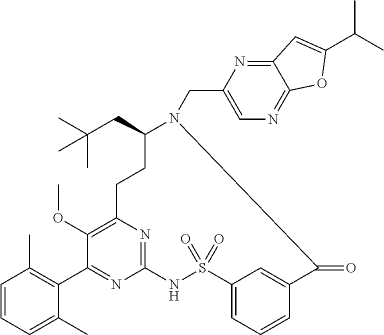 [SMILES: COc1c2nc(nc1-c1c(C)cccc1C)NS(=O)(=O)c1cccc(c1)C(=O)N(Cc1cnc3oc(C(C)C)cc3n1)[C@H](CC(C)(C)C)CC2] |
| I-158 | 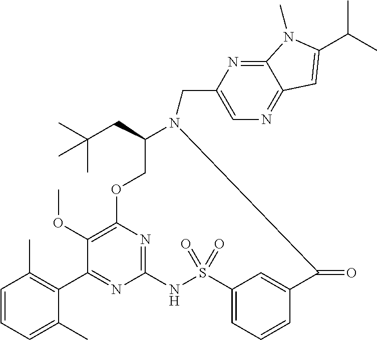 [SMILES: COc1c2nc(nc1-c1c(C)cccc1C)NS(=O)(=O)c1cccc(c1)C(=O)N(Cc1cnc3cc(C(C)C)n(C)c3n1)[C@H](CC(C)(C)C)CO2] |
| I-159 | 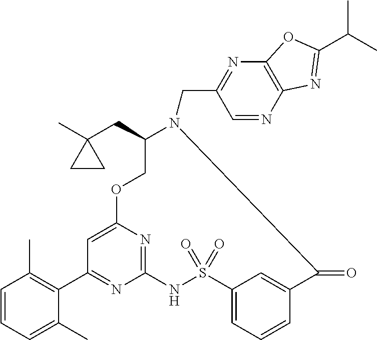 [SMILES: Cc1cccc(C)c1-c1cc2nc(n1)NS(=O)(=O)c1cccc(c1)C(=O)N(Cc1cnc3nc(C(C)C)oc3n1)[C@H](CC1(C)CC1)CO2] |
| I-160 | 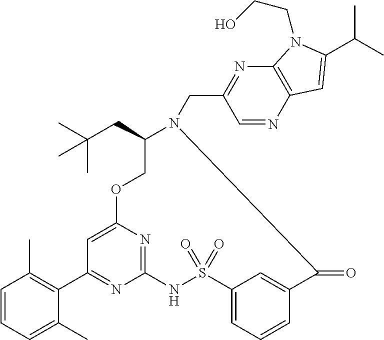 [SMILES: Cc1cccc(C)c1-c1cc2nc(n1)NS(=O)(=O)c1cccc(c1)C(=O)N(Cc1cnc3cc(C(C)C)n(CCO)c3n1)[C@H](CC(C)(C)C)CO2] |
| I-161 | 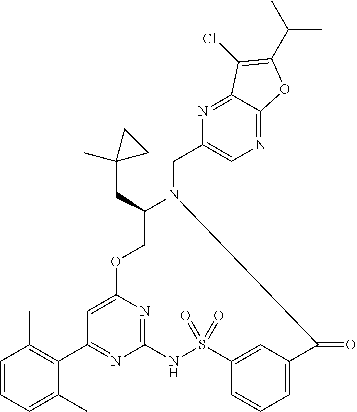 [SMILES: Cc1cccc(C)c1-c1cc2nc(n1)NS(=O)(=O)c1cccc(c1)C(=O)N(Cc1cnc3oc(C(C)C)c(Cl)c3n1)[C@H](CC1(C)CC1)CO2] |
| I-162 | 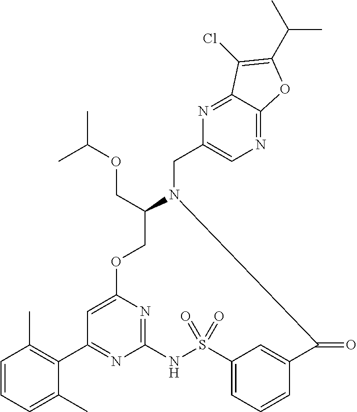 [SMILES: Cc1cccc(C)c1-c1cc2nc(n1)NS(=O)(=O)c1cccc(c1)C(=O)N(Cc1cnc3oc(C(C)C)c(Cl)c3n1)[C@H](COC(C)C)CO2] |
| I-163 | 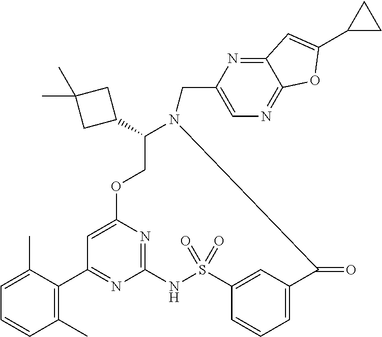 [SMILES: Cc1cccc(C)c1-c1cc2nc(n1)NS(=O)(=O)c1cccc(c1)C(=O)N(Cc1cnc3oc(C4CC4)cc3n1)[C@@H](C1CC(C)(C)C1)CO2] |
| I-164 | 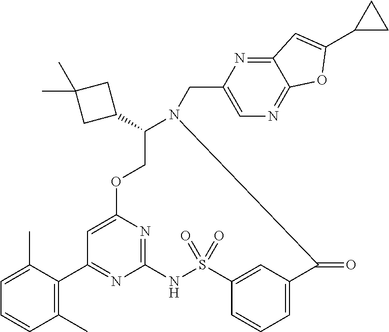 [SMILES: Cc1cccc(C)c1-c1cc2nc(n1)NS(=O)(=O)c1cccc(c1)C(=O)N(Cc1cnc3oc(C4CC4)cc3n1)[C@@H](C1CC(C)(C)C1)CO2] |
| I-165 | 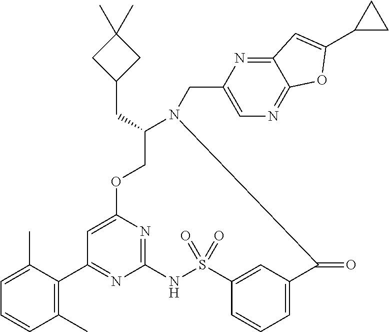 [SMILES: Cc1cccc(C)c1-c1cc2nc(n1)NS(=O)(=O)c1cccc(c1)C(=O)N(Cc1cnc3oc(C4CC4)cc3n1)[C@@H](CC1CC(C)(C)C1)CO2] |
| I-166 | 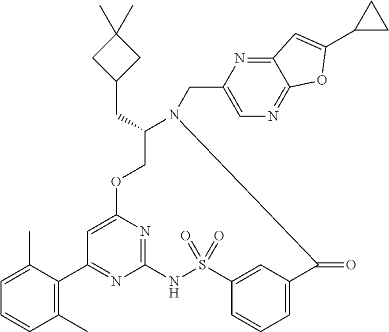 [SMILES: Cc1cccc(C)c1-c1cc2nc(n1)NS(=O)(=O)c1cccc(c1)C(=O)N(Cc1cnc3oc(C4CC4)cc3n1)[C@@H](CC1CC(C)(C)C1)CO2] |
| I-167 | 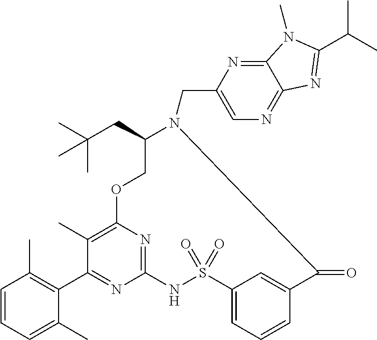 [SMILES: Cc1cccc(C)c1-c1nc2nc(c1C)OC[C@@H](CC(C)(C)C)N(Cc1cnc3nc(C(C)C)n(C)c3n1)C(=O)c1cccc(c1)S(=O)(=O)N2] |
| I-168 | 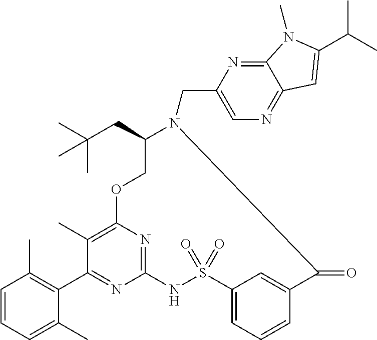 [SMILES: Cc1cccc(C)c1-c1nc2nc(c1C)OC[C@@H](CC(C)(C)C)N(Cc1cnc3cc(C(C)C)n(C)c3n1)C(=O)c1cccc(c1)S(=O)(=O)N2] |
| I-169 | 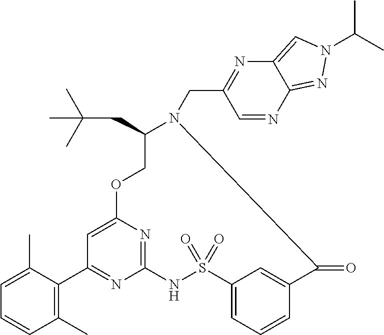 [SMILES: Cc1cccc(C)c1-c1cc2nc(n1)NS(=O)(=O)c1cccc(c1)C(=O)N(Cc1cnc3nn(C(C)C)cc3n1)[C@H](CC(C)(C)C)CO2] |
| I-170 | 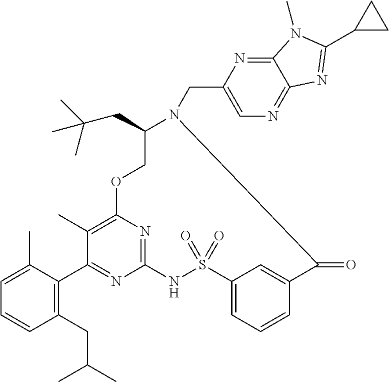 [SMILES: Cc1cccc(CC(C)C)c1-c1nc2nc(c1C)OC[C@@H](CC(C)(C)C)N(Cc1cnc3nc(C4CC4)n(C)c3n1)C(=O)c1cccc(c1)S(=O)(=O)N2] |
| I-171 | 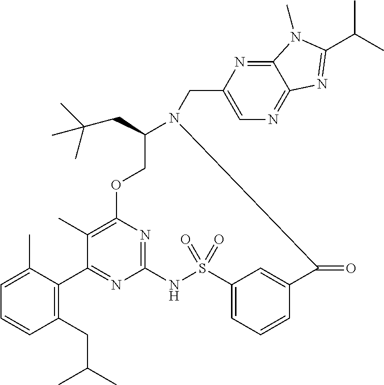 [SMILES: Cc1cccc(CC(C)C)c1-c1nc2nc(c1C)OC[C@@H](CC(C)(C)C)N(Cc1cnc3nc(C(C)C)n(C)c3n1)C(=O)c1cccc(c1)S(=O)(=O)N2] |
| I-172 | 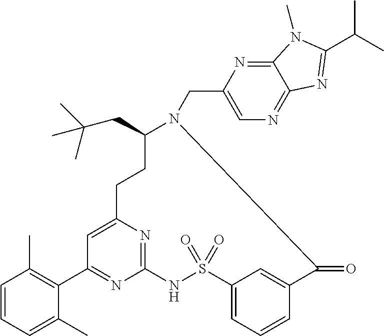 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CC2] |
| I-173 | 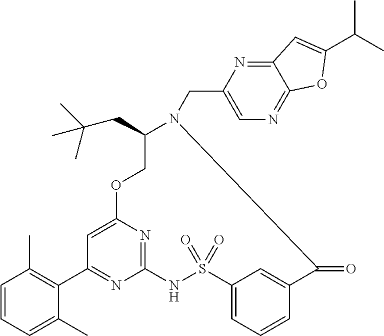 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2] |
| I-174 | 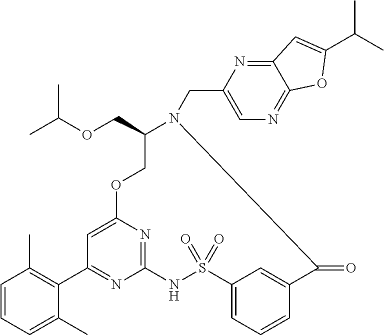 [SMILES: Cc1cccc(C)c1-c1cc2nc(n1)NS(=O)(=O)c1cccc(c1)C(=O)N(Cc1cnc3oc(C(C)C)cc3n1)[C@H](COC(C)C)CO2] |
| I-175 | 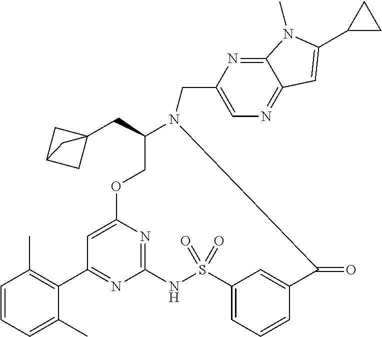 [SMILES: Cc1cccc(C)c1-c1cc2nc(n1)NS(=O)(=O)c1cccc(c1)C(=O)N(Cc1cnc3cc(C4CC4)n(C)c3n1)[C@H](CC13CC(C1)C3)CO2] |
| I-176 | 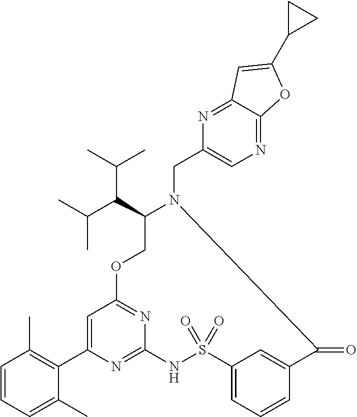 [SMILES: Cc1cccc(C)c1-c1cc2nc(n1)NS(=O)(=O)c1cccc(c1)C(=O)N(Cc1cnc3oc(C4CC4)cc3n1)[C@H](C(C(C)C)C(C)C)CO2] |
| I-177 | 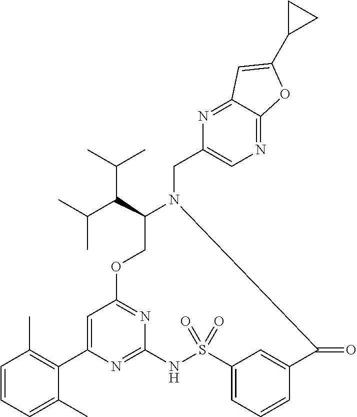 [SMILES: Cc1cccc(C)c1-c1cc2nc(n1)NS(=O)(=O)c1cccc(c1)C(=O)N(Cc1cnc3oc(C4CC4)cc3n1)[C@H](C(C(C)C)C(C)C)CO2] |
| I-178 | 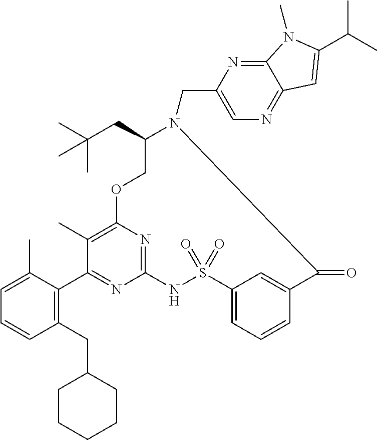 [SMILES: Cc1cccc(CC2CCCCC2)c1-c1nc2nc(c1C)OC[C@@H](CC(C)(C)C)N(Cc1cnc3cc(C(C)C)n(C)c3n1)C(=O)c1cccc(c1)S(=O)(=O)N2] |
| I-179 | 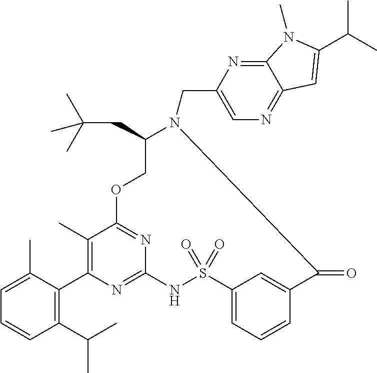 [SMILES: Cc1cccc(C(C)C)c1-c1nc2nc(c1C)OC[C@@H](CC(C)(C)C)N(Cc1cnc3cc(C(C)C)n(C)c3n1)C(=O)c1cccc(c1)S(=O)(=O)N2] |
| I-180 | 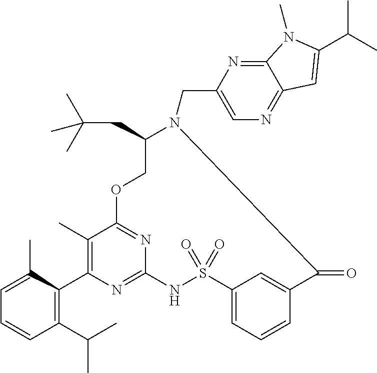 |
| I-181 | 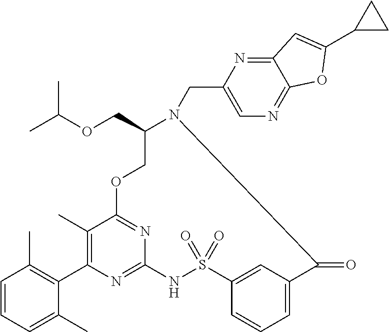 [SMILES: Cc1cccc(C)c1-c1nc2nc(c1C)OC[C@@H](COC(C)C)N(Cc1cnc3oc(C4CC4)cc3n1)C(=O)c1cccc(c1)S(=O)(=O)N2] |
| I-182 | 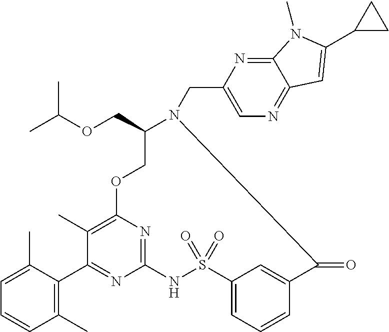 [SMILES: Cc1cccc(C)c1-c1nc2nc(c1C)OC[C@@H](COC(C)C)N(Cc1cnc3cc(C4CC4)n(C)c3n1)C(=O)c1cccc(c1)S(=O)(=O)N2] |
| I-183 | 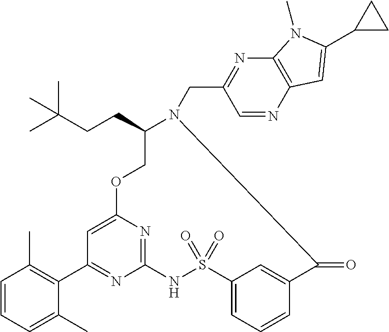 [SMILES: Cc1cccc(C)c1-c1cc2nc(n1)NS(=O)(=O)c1cccc(c1)C(=O)N(Cc1cnc3cc(C4CC4)n(C)c3n1)[C@H](CCC(C)(C)C)CO2] |
| I-184 | 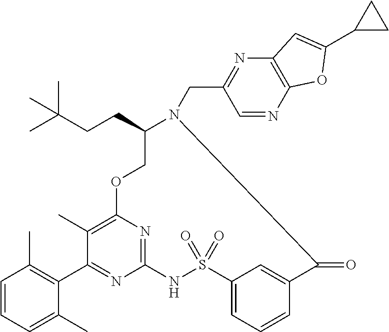 [SMILES: Cc1cccc(C)c1-c1nc2nc(c1C)OC[C@@H](CCC(C)(C)C)N(Cc1cnc3oc(C4CC4)cc3n1)C(=O)c1cccc(c1)S(=O)(=O)N2] |
| I-185 | 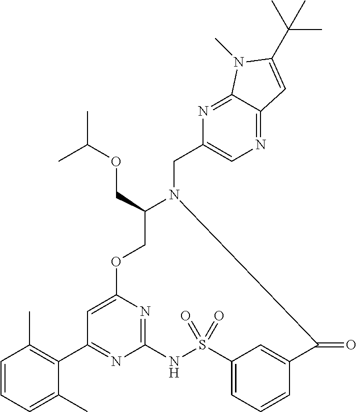 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](COC(C)C)CO2] |
| I-186 | 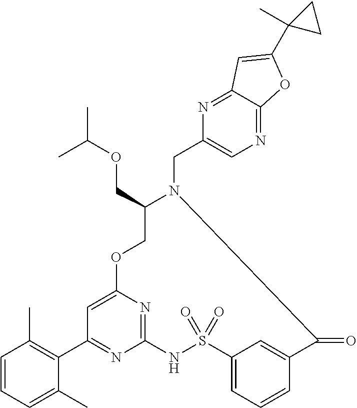 [SMILES: Cc1cccc(C)c1-c1cc2nc(n1)NS(=O)(=O)c1cccc(c1)C(=O)N(Cc1cnc3oc(C4(C)CC4)cc3n1)[C@H](COC(C)C)CO2] |
| I-187 | 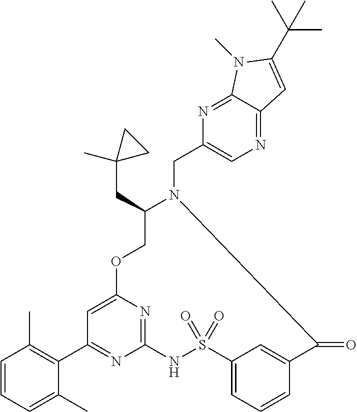 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](CC1(C)CC1)CO2] |
| I-188 | 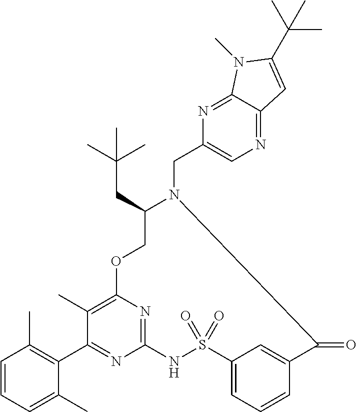 [SMILES: Cc1cccc(C)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2] |
| I-189 | 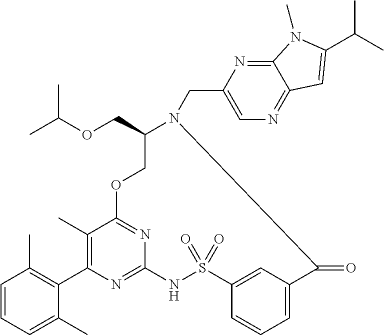 [SMILES: Cc1cccc(C)c1-c1nc2nc(c1C)OC[C@@H](COC(C)C)N(Cc1cnc3cc(C(C)C)n(C)c3n1)C(=O)c1cccc(c1)S(=O)(=O)N2] |
| I-190 | 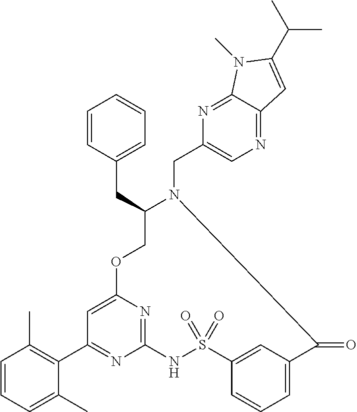 [SMILES: Cc1cccc(C)c1-c1cc2nc(n1)NS(=O)(=O)c1cccc(c1)C(=O)N(Cc1cnc3cc(C(C)C)n(C)c3n1)[C@H](Cc1ccccc1)CO2] |
| I-191 | 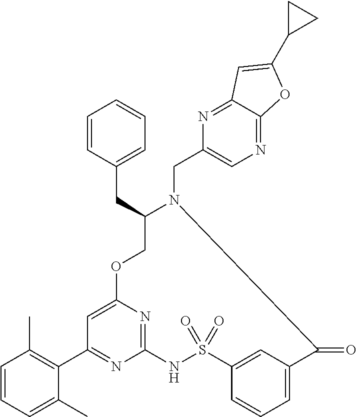 [SMILES: Cc1cccc(C)c1-c1cc2nc(n1)NS(=O)(=O)c1cccc(c1)C(=O)N(Cc1cnc3oc(C4CC4)cc3n1)[C@H](Cc1ccccc1)CO2] |
| I-192 | 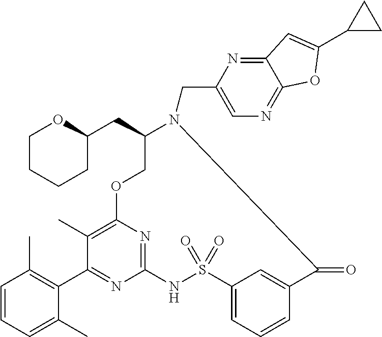 [SMILES: Cc1cccc(C)c1-c1nc2nc(c1C)OC[C@@H](C[C@H]1CCCCO1)N(Cc1cnc3oc(C4CC4)cc3n1)C(=O)c1cccc(c1)S(=O)(=O)N2] |
| I-193 | 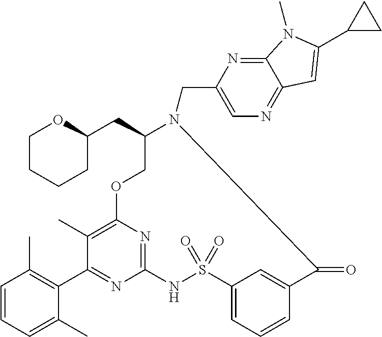 [SMILES: Cc1cccc(C)c1-c1nc2nc(c1C)OC[C@@H](C[C@H]1CCCCO1)N(Cc1cnc3cc(C4CC4)n(C)c3n1)C(=O)c1cccc(c1)S(=O)(=O)N2] |
| I-194 | 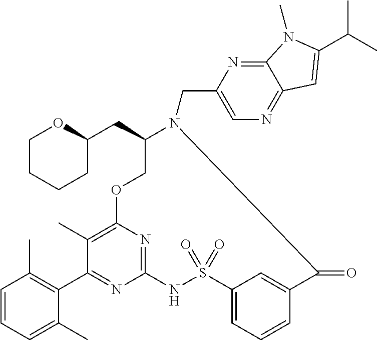 [SMILES: Cc1cccc(C)c1-c1nc2nc(c1C)OC[C@@H](C[C@H]1CCCCO1)N(Cc1cnc3cc(C(C)C)n(C)c3n1)C(=O)c1cccc(c1)S(=O)(=O)N2] |
| I-195 | 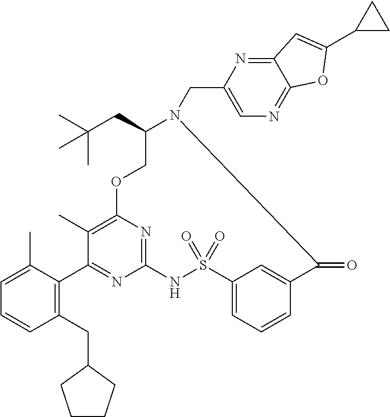 [SMILES: Cc1cccc(CC2CCCC2)c1-c1nc2nc(c1C)OC[C@@H](CC(C)(C)C)N(Cc1cnc3oc(C4CC4)cc3n1)C(=O)c1cccc(c1)S(=O)(=O)N2] |
| I-196 | 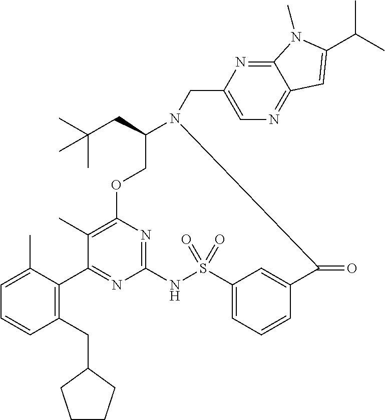 [SMILES: Cc1cccc(CC2CCCC2)c1-c1nc2nc(c1C)OC[C@@H](CC(C)(C)C)N(Cc1cnc3cc(C(C)C)n(C)c3n1)C(=O)c1cccc(c1)S(=O)(=O)N2] |
| I-197 | 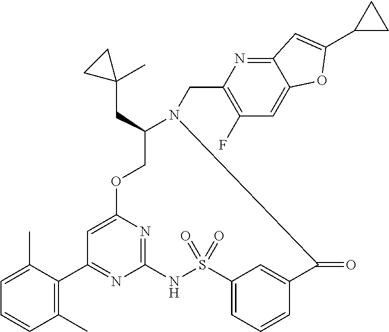 [SMILES: Cc1cccc(C)c1-c1cc2nc(n1)NS(=O)(=O)c1cccc(c1)C(=O)N(Cc1nc3cc(C4CC4)oc3cc1F)[C@H](CC1(C)CC1)CO2] |
| I-198 | 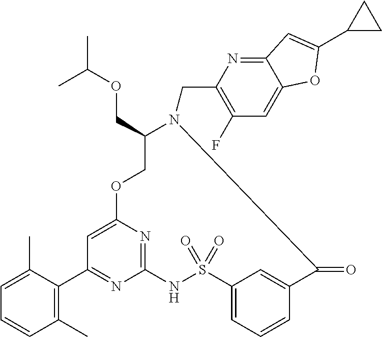 [SMILES: Cc1cccc(C)c1-c1cc2nc(n1)NS(=O)(=O)c1cccc(c1)C(=O)N(Cc1nc3cc(C4CC4)oc3cc1F)[C@H](COC(C)C)CO2] |
| I-199 | 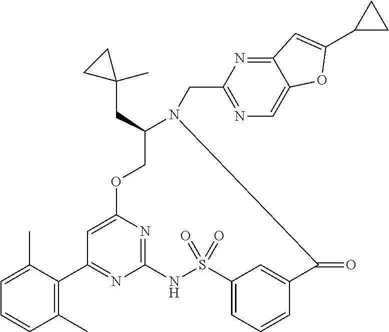 [SMILES: Cc1cccc(C)c1-c1cc2nc(n1)NS(=O)(=O)c1cccc(c1)C(=O)N(Cc1ncc3oc(C4CC4)cc3n1)[C@H](CC1(C)CC1)CO2] |
| I-200 | 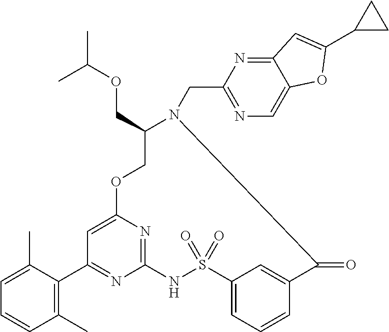 [SMILES: Cc1cccc(C)c1-c1cc2nc(n1)NS(=O)(=O)c1cccc(c1)C(=O)N(Cc1ncc3oc(C4CC4)cc3n1)[C@H](COC(C)C)CO2] |
| I-202 | 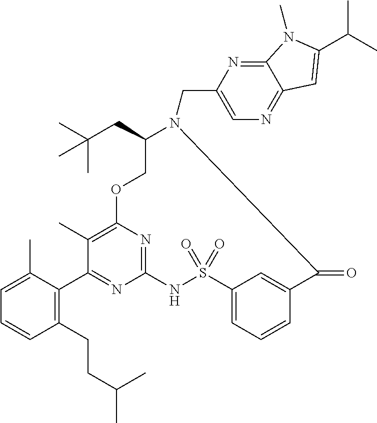 [SMILES: Cc1cccc(CCC(C)C)c1-c1nc2nc(c1C)OC[C@@H](CC(C)(C)C)N(Cc1cnc3cc(C(C)C)n(C)c3n1)C(=O)c1cccc(c1)S(=O)(=O)N2] |
| I-203 | 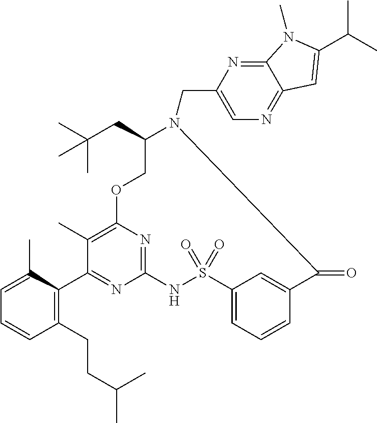 |
| I-204 | 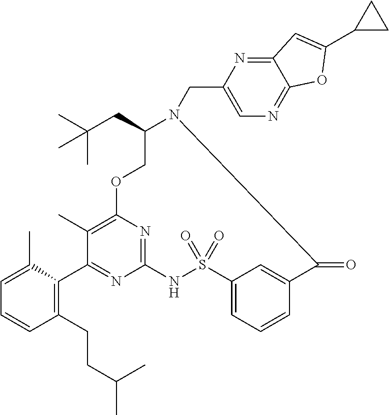 |
| I-205 | 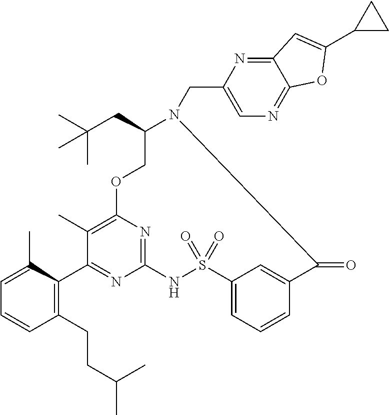 |
| I-206 | 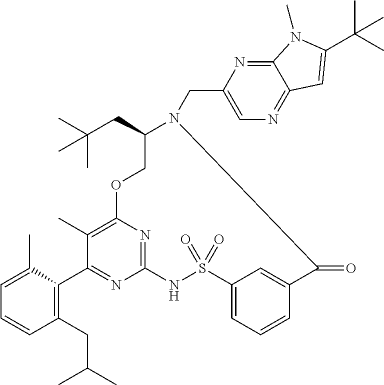 |
| I-207 | 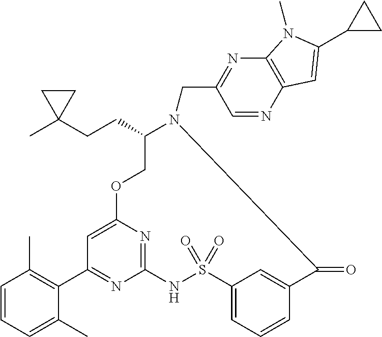 [SMILES: Cc1cccc(C)c1-c1cc2nc(n1)NS(=O)(=O)c1cccc(c1)C(=O)N(Cc1cnc3cc(C4CC4)n(C)c3n1)[C@@H](CCC1(C)CC1)CO2] |
| I-208 | 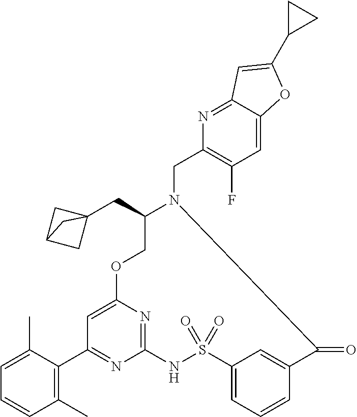 [SMILES: Cc1cccc(C)c1-c1cc2nc(n1)NS(=O)(=O)c1cccc(c1)C(=O)N(Cc1nc3cc(C4CC4)oc3cc1F)[C@H](CC13CC(C1)C3)CO2] |
| I-209 | 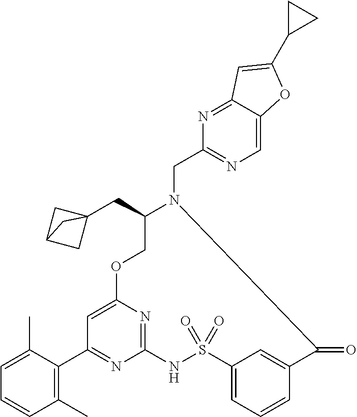 [SMILES: Cc1cccc(C)c1-c1cc2nc(n1)NS(=O)(=O)c1cccc(c1)C(=O)N(Cc1ncc3oc(C4CC4)cc3n1)[C@H](CC13CC(C1)C3)CO2] |
| I-210 | 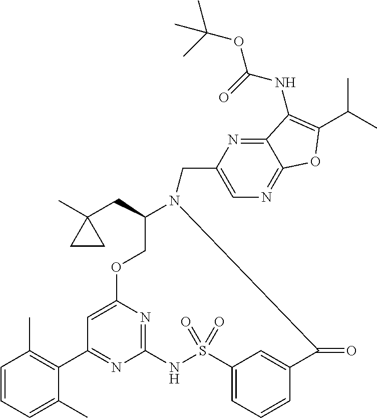 [SMILES: Cc1cccc(C)c1-c1cc2nc(n1)NS(=O)(=O)c1cccc(c1)C(=O)N(Cc1cnc3oc(C(C)C)c(NC(=O)OC(C)(C)C)c3n1)[C@H](CC1(C)CC1)CO2] |
| I-211 | 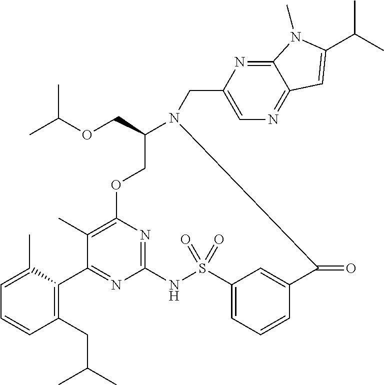 |
| I-212 | 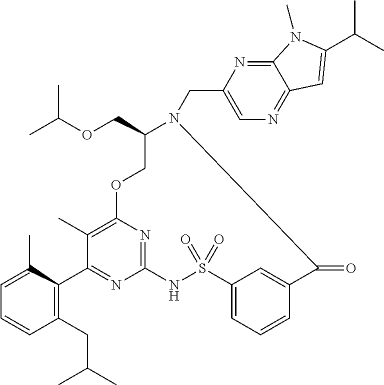 |
| I-213 | 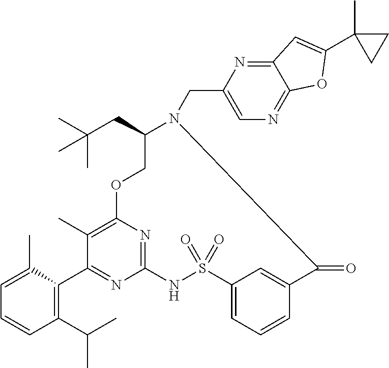 |
| I-214 | 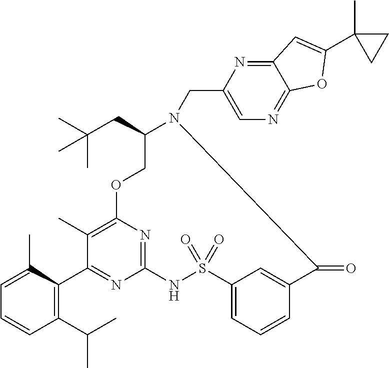 |
| I-215 | 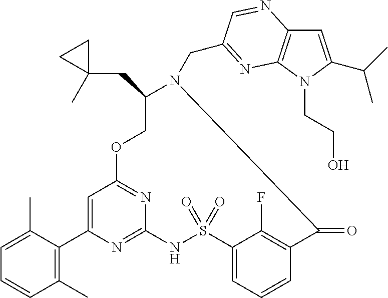 [SMILES: Cc1cccc(C)c1-c1cc2nc(n1)NS(=O)(=O)c1cccc(c1F)C(=O)N(Cc1cnc3cc(C(C)C)n(CCO)c3n1)[C@H](CC1(C)CC1)CO2] |
| I-216 | 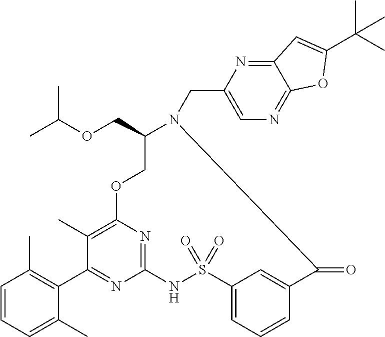 [SMILES: Cc1cccc(C)c1-c1nc2nc(c1C)OC[C@@H](COC(C)C)N(Cc1cnc3oc(C(C)(C)C)cc3n1)C(=O)c1cccc(c1)S(=O)(=O)N2] |
| I-217 | 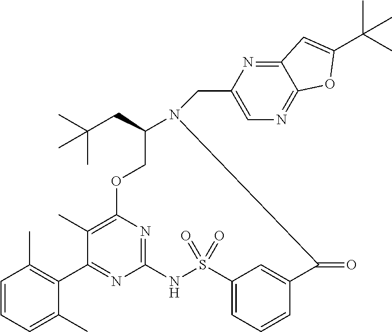 [SMILES: Cc1cccc(C)c1-c1nc2nc(c1C)OC[C@@H](CC(C)(C)C)N(Cc1cnc3oc(C(C)(C)C)cc3n1)C(=O)c1cccc(c1)S(=O)(=O)N2] |
| I-218 | 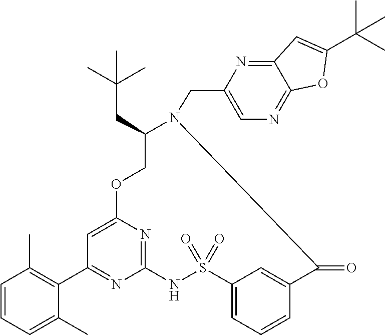 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC(C)(C)C)CO2] |
| I-219 | 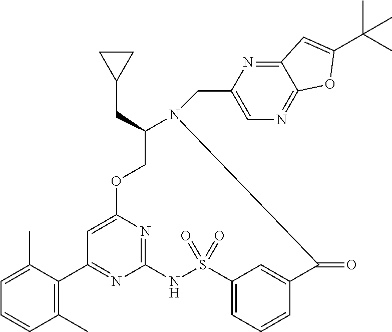 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CC1)CO2] |
| I-220 | 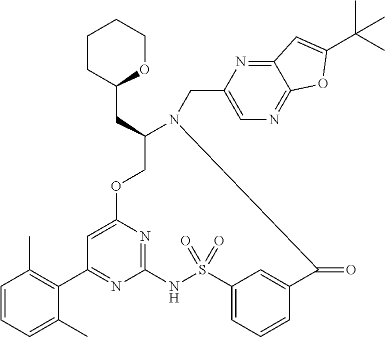 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@H]1CCCCO1)CO2] |
| I-221 | 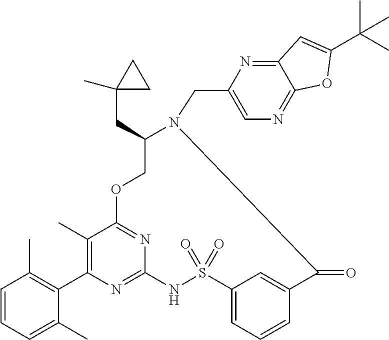 [SMILES: Cc1cccc(C)c1-c1nc2nc(c1C)OC[C@@H](CC1(C)CC1)N(Cc1cnc3oc(C(C)(C)C)cc3n1)C(=O)c1cccc(c1)S(=O)(=O)N2] |
| I-222 | 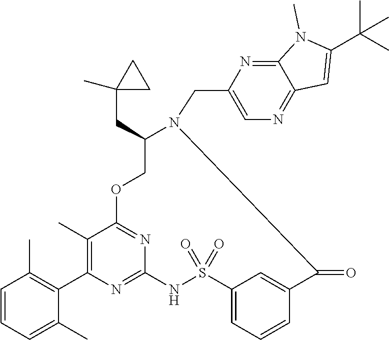 [SMILES: Cc1cccc(C)c1-c1nc2nc(c1C)OC[C@@H](CC1(C)CC1)N(Cc1cnc3cc(C(C)(C)C)n(C)c3n1)C(=O)c1cccc(c1)S(=O)(=O)N2] |
| I-223 | 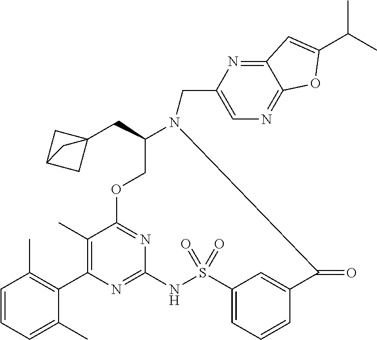 [SMILES: Cc1cccc(C)c1-c1nc2nc(c1C)OC[C@@H](CC13CC(C1)C3)N(Cc1cnc3oc(C(C)C)cc3n1)C(=O)c1cccc(c1)S(=O)(=O)N2] |
| I-224 | 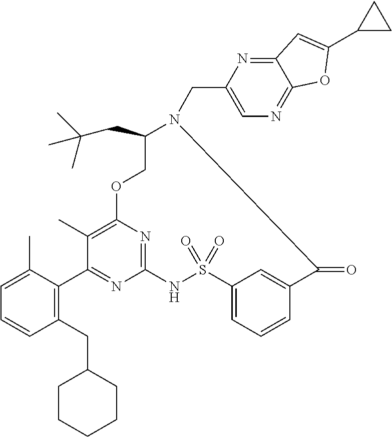 [SMILES: Cc1cccc(CC2CCCCC2)c1-c1nc2nc(c1C)OC[C@@H](CC(C)(C)C)N(Cc1cnc3oc(C4CC4)cc3n1)C(=O)c1cccc(c1)S(=O)(=O)N2] |
| I-225 | 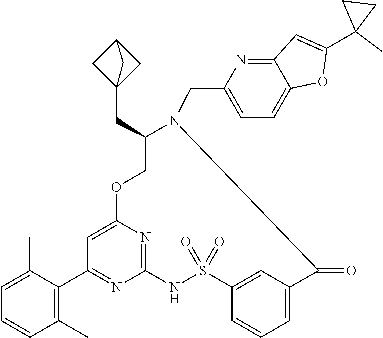 [SMILES: Cc1cccc(C)c1-c1cc2nc(n1)NS(=O)(=O)c1cccc(c1)C(=O)N(Cc1ccc3oc(C4(C)CC4)cc3n1)[C@H](CC13CC(C1)C3)CO2] |
| I-226 | 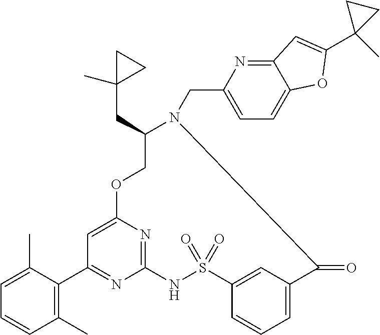 [SMILES: Cc1cccc(C)c1-c1cc2nc(n1)NS(=O)(=O)c1cccc(c1)C(=O)N(Cc1ccc3oc(C4(C)CC4)cc3n1)[C@H](CC1(C)CC1)CO2] |
| I-227 | 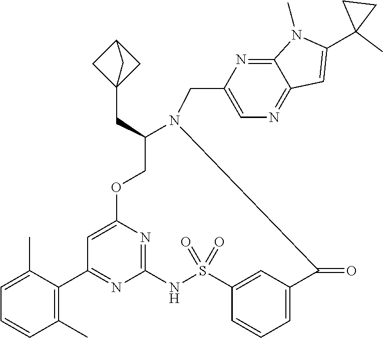 [SMILES: Cc1cccc(C)c1-c1cc2nc(n1)NS(=O)(=O)c1cccc(c1)C(=O)N(Cc1cnc3cc(C4(C)CC4)n(C)c3n1)[C@H](CC13CC(C1)C3)CO2] |
| I-228 | 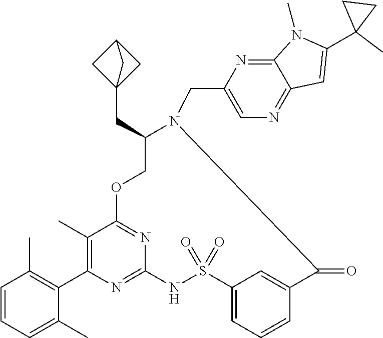 [SMILES: Cc1cccc(C)c1-c1nc2nc(c1C)OC[C@@H](CC13CC(C1)C3)N(Cc1cnc3cc(C4(C)CC4)n(C)c3n1)C(=O)c1cccc(c1)S(=O)(=O)N2] |
| I-229 | 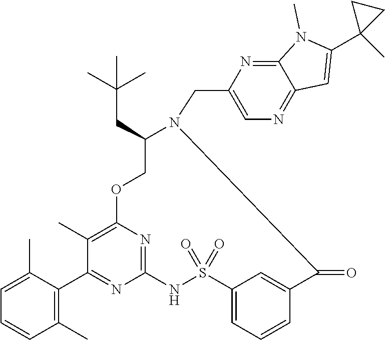 [SMILES: Cc1cccc(C)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] |
| I-230 | 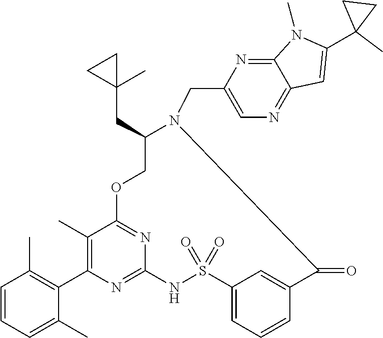 [SMILES: Cc1cccc(C)c1-c1nc2nc(c1C)OC[C@@H](CC1(C)CC1)N(Cc1cnc3cc(C4(C)CC4)n(C)c3n1)C(=O)c1cccc(c1)S(=O)(=O)N2] |
| I-231 | 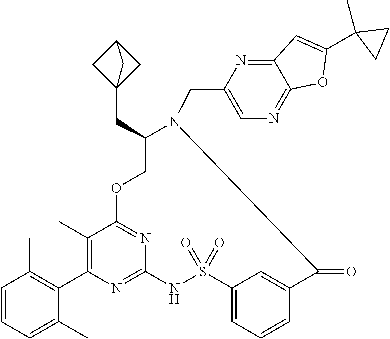 [SMILES: Cc1cccc(C)c1-c1nc2nc(c1C)OC[C@@H](CC13CC(C1)C3)N(Cc1cnc3oc(C4(C)CC4)cc3n1)C(=O)c1cccc(c1)S(=O)(=O)N2] |
| I-232 | 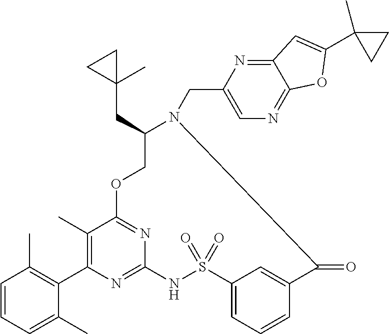 [SMILES: Cc1cccc(C)c1-c1nc2nc(c1C)OC[C@@H](CC1(C)CC1)N(Cc1cnc3oc(C4(C)CC4)cc3n1)C(=O)c1cccc(c1)S(=O)(=O)N2] |
| I-233 | 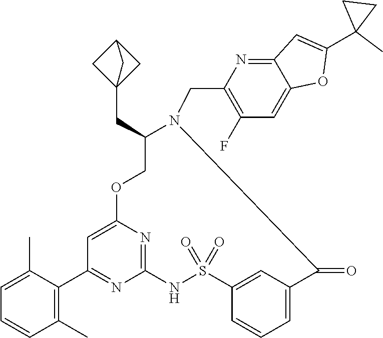 [SMILES: Cc1cccc(C)c1-c1cc2nc(n1)NS(=O)(=O)c1cccc(c1)C(=O)N(Cc1nc3cc(C4(C)CC4)oc3cc1F)[C@H](CC13CC(C1)C3)CO2] |
| I-234 | 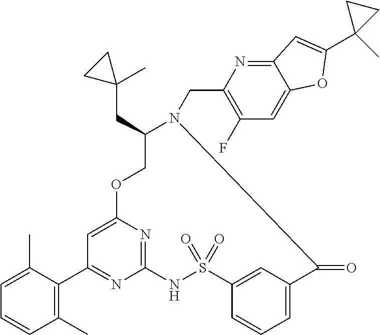 [SMILES: Cc1cccc(C)c1-c1cc2nc(n1)NS(=O)(=O)c1cccc(c1)C(=O)N(Cc1nc3cc(C4(C)CC4)oc3cc1F)[C@H](CC1(C)CC1)CO2] |
| I-235 | 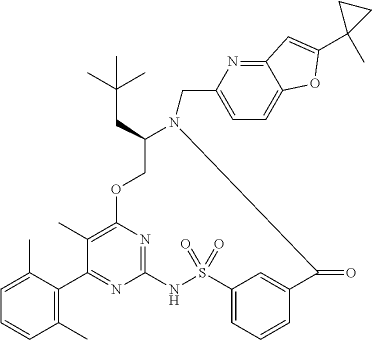 [SMILES: Cc1cccc(C)c1-c1nc2nc(c1C)OC[C@@H](CC(C)(C)C)N(Cc1ccc3oc(C4(C)CC4)cc3n1)C(=O)c1cccc(c1)S(=O)(=O)N2] |
| I-236 | 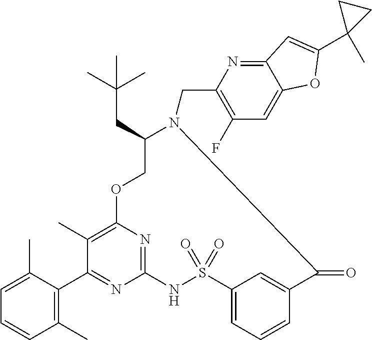 [SMILES: Cc1cccc(C)c1-c1nc2nc(c1C)OC[C@@H](CC(C)(C)C)N(Cc1nc3cc(C4(C)CC4)oc3cc1F)C(=O)c1cccc(c1)S(=O)(=O)N2] |
| I-237 | 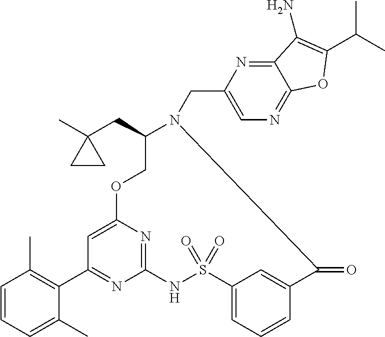 [SMILES: Cc1cccc(C)c1-c1cc2nc(n1)NS(=O)(=O)c1cccc(c1)C(=O)N(Cc1cnc3oc(C(C)C)c(N)c3n1)[C@H](CC1(C)CC1)CO2] |
| I-238 | 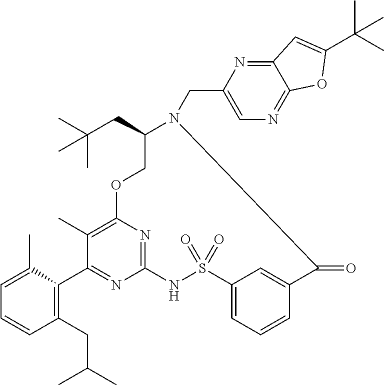 |
| I-239 | 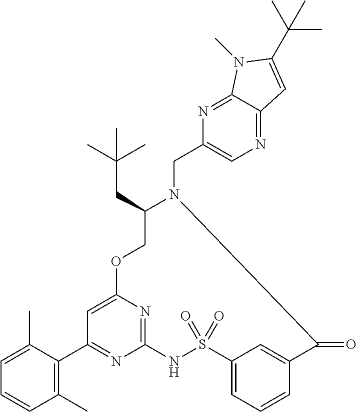 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](CC(C)(C)C)CO2] |
| I-240 | 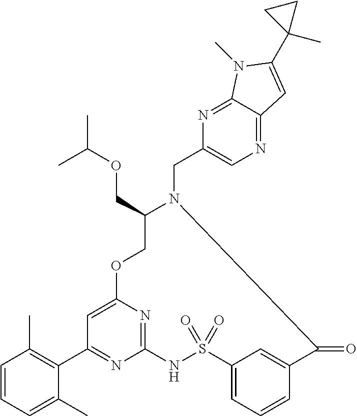 [SMILES: Cc1cccc(C)c1-c1cc2nc(n1)NS(=O)(=O)c1cccc(c1)C(=O)N(Cc1cnc3cc(C4(C)CC4)n(C)c3n1)[C@H](COC(C)C)CO2] |
| I-241 | 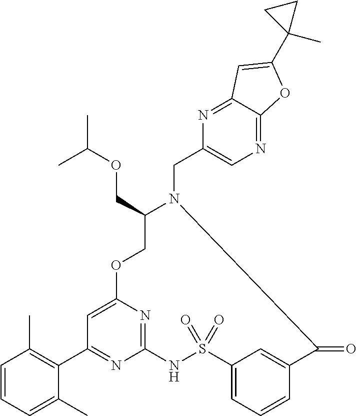 [SMILES: Cc1cccc(C)c1-c1cc2nc(n1)NS(=O)(=O)c1cccc(c1)C(=O)N(Cc1cnc3oc(C4(C)CC4)cc3n1)[C@H](COC(C)C)CO2] |
| I-242 | 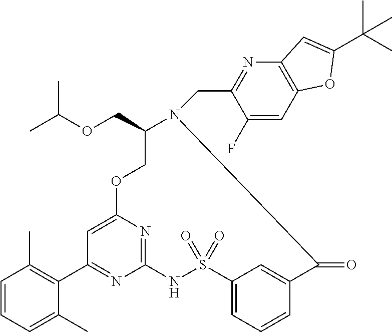 [SMILES: Cc1cccc(C)c1-c1cc2nc(n1)NS(=O)(=O)c1cccc(c1)C(=O)N(Cc1nc3cc(C(C)(C)C)oc3cc1F)[C@H](COC(C)C)CO2] |
| I-243 | 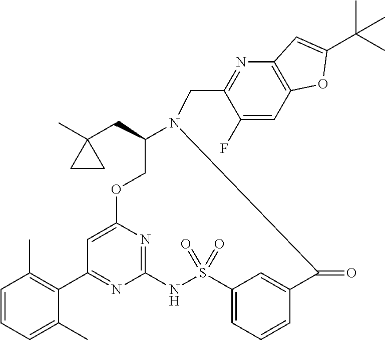 [SMILES: Cc1cccc(C)c1-c1cc2nc(n1)NS(=O)(=O)c1cccc(c1)C(=O)N(Cc1nc3cc(C(C)(C)C)oc3cc1F)[C@H](CC1(C)CC1)CO2] |
| I-244 | 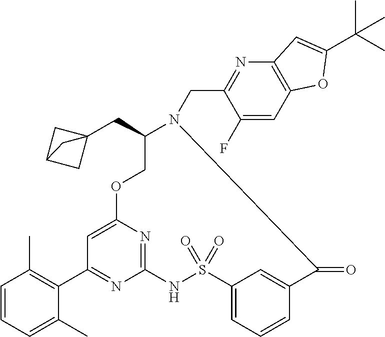 [SMILES: Cc1cccc(C)c1-c1cc2nc(n1)NS(=O)(=O)c1cccc(c1)C(=O)N(Cc1nc3cc(C(C)(C)C)oc3cc1F)[C@H](CC13CC(C1)C3)CO2] |
| I-245 | 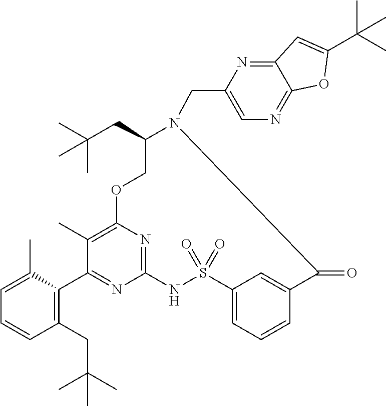 |
| I-246 | 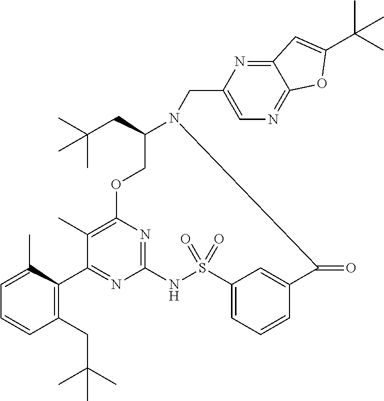 |
| I-247 | 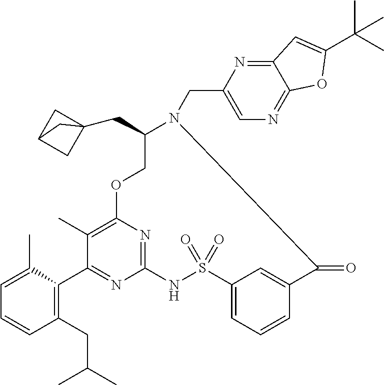 |
| I-248 | 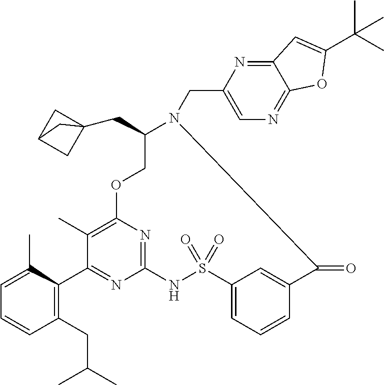 |
| I-249 | 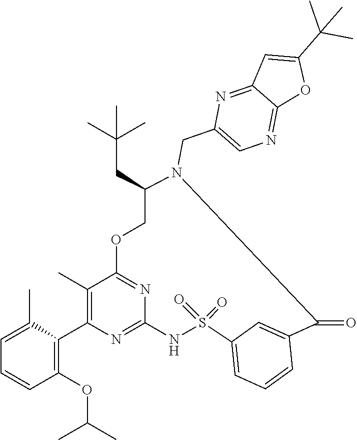 |
| I-250 | 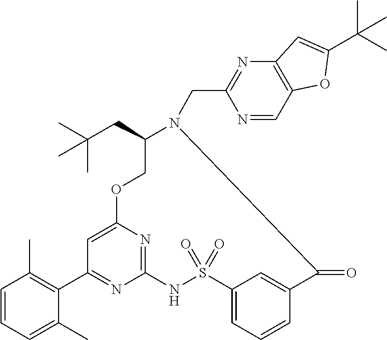 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC(C)(C)C)CO2] |
| I-251 | 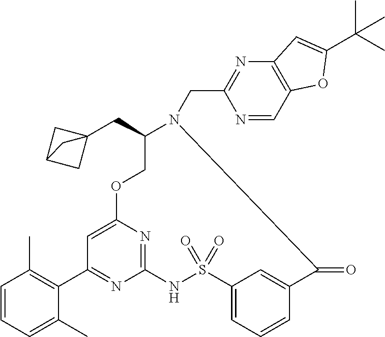 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC13CC(C1)C3)CO2] |
| I-252 | 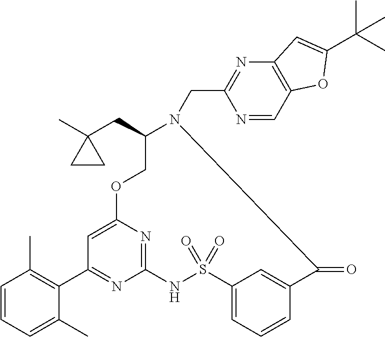 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC1(C)CC1)CO2] |
| I-253 | 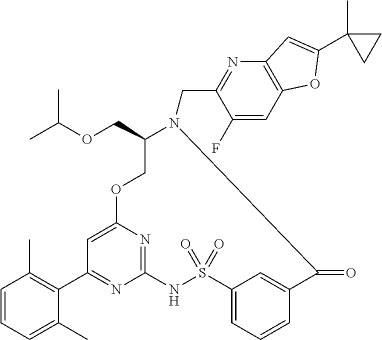 [SMILES: Cc1cccc(C)c1-c1cc2nc(n1)NS(=O)(=O)c1cccc(c1)C(=O)N(Cc1nc3cc(C4(C)CC4)oc3cc1F)[C@H](COC(C)C)CO2] |
| I-254 | 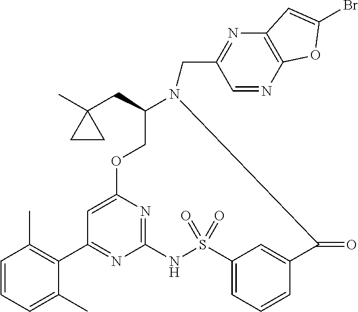 [SMILES: Cc1cccc(C)c1-c1cc2nc(n1)NS(=O)(=O)c1cccc(c1)C(=O)N(Cc1cnc3oc(Br)cc3n1)[C@H](CC1(C)CC1)CO2] |
| I-255 | 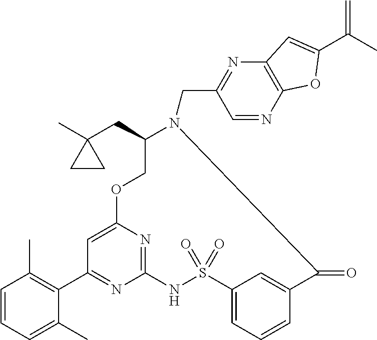 [SMILES: C=C(C)c1cc2nc(CN3C(=O)c4cccc(c4)S(=O)(=O)Nc4nc(cc(-c5c(C)cccc5C)n4)OC[C@H]3CC3(C)CC3)cnc2o1] |
| I-256 | 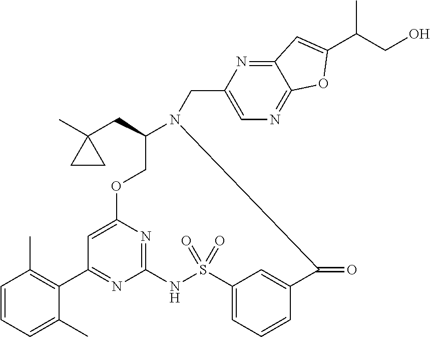 [SMILES: Cc1cccc(C)c1-c1cc2nc(n1)NS(=O)(=O)c1cccc(c1)C(=O)N(Cc1cnc3oc(C(C)CO)cc3n1)[C@H](CC1(C)CC1)CO2] |
| I-257 | 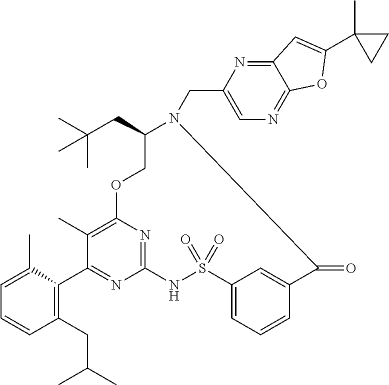 |
| I-258 | 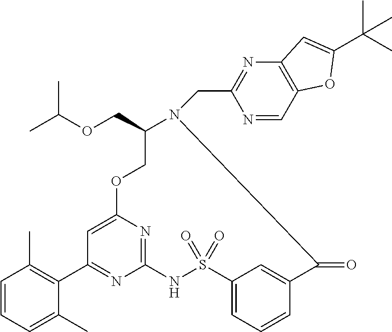 [SMILES: Cc1cccc(C)c1-c1cc2nc(n1)NS(=O)(=O)c1cccc(c1)C(=O)N(Cc1ncc3oc(C(C)(C)C)cc3n1)[C@H](COC(C)C)CO2] |
| I-259 | 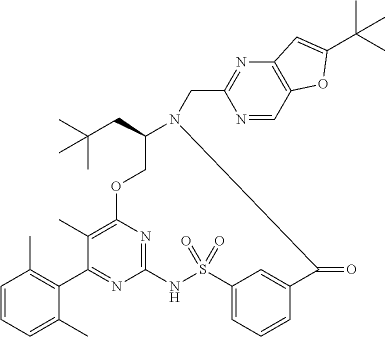 [SMILES: Cc1cccc(C)c1-c1nc2nc(c1C)OC[C@@H](CC(C)(C)C)N(Cc1ncc3oc(C(C)(C)C)cc3n1)C(=O)c1cccc(c1)S(=O)(=O)N2] |
| I-260 | 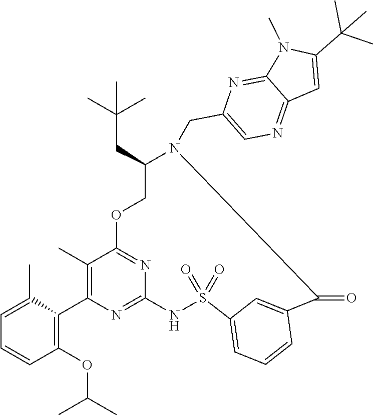 |
| I-261 | 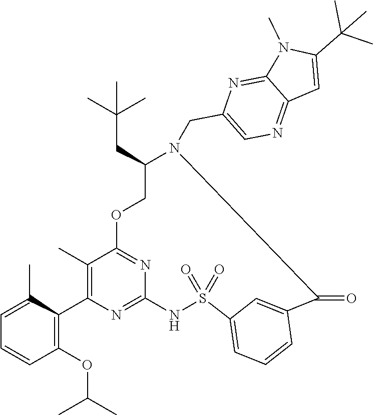 |
| I-262 | 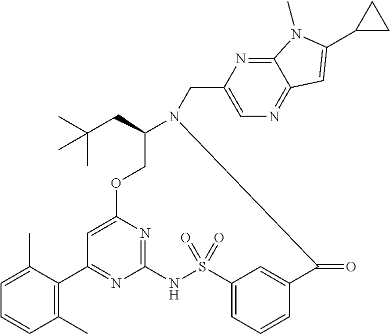 [SMILES: Cc1cccc(C)c1-c1cc2nc(n1)NS(=O)(=O)c1cccc(c1)C(=O)N(Cc1cnc3cc(C4CC4)n(C)c3n1)[C@H](CC(C)(C)C)CO2] |
| I-263 | 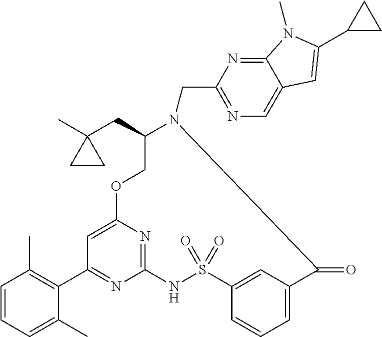 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC1(C)CC1)CO2] |
| I-264 | 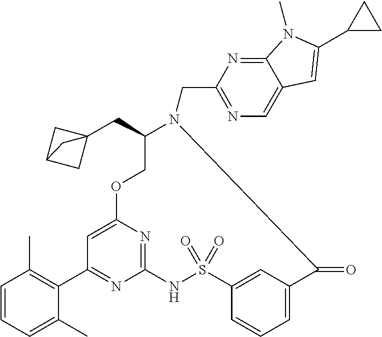 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC13CC(C1)C3)CO2] |
| I-265 | 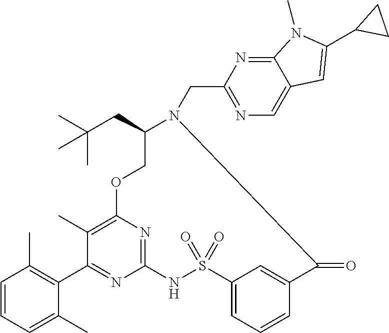 [SMILES: Cc1cccc(C)c1-c1nc2nc(c1C)OC[C@@H](CC(C)(C)C)N(Cc1ncc3cc(C4CC4)n(C)c3n1)C(=O)c1cccc(c1)S(=O)(=O)N2] |

In some embodiments, the disclosure provides compounds of Formula II:

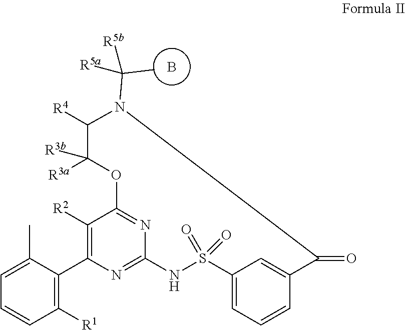

or a tautomer thereof, or a deuterated derivative of the compound or tautomer, or a pharmaceutically acceptable salt of any of the foregoing, wherein:

Ring B is a 6-membered heteroaryl, optionally substituted with 1 to 2 groups independently selected from
halogen
4- to 10-membered heterocyclyl (which may be optionally substituted with 1 to 3 groups independently selected from halogen, oxo, C₁-C₄ alkyl)
N(R^{x})₂, wherein R^{x} is independently selected from hydrogen, C₁-C₄ alkyl, C₃-C₆ cycloalkyl (which may be optionally substituted with a group selected from halogen, C₁-C₄ haloalkyl, and C₁-C₄ alkyl)
C₁-C₄ alkyl (optionally substituted with C₃-C₆ cycloalkyl (which may be further optionally substituted with a group selected from halogen, —OH))
R¹ is selected from: C₃-C₆ cycloalkyl, C₁-C₆ alkoxy, and C₁-C₆ alkyl (which may be optionally substituted with a group selected from C₄-C₆ cycloalkyl, C₄-C₆ aryl, 4- to 6-membered heterocyclyl, and 4- to 6-membered heteroaryl);
R² is selected from: hydrogen, halogen, C₁-C₈ alkyl, haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl).

In some embodiments, Ring B in the compound, tautomer, deuterated derivative, or salt of Formula II is a heteroaryl selected from:

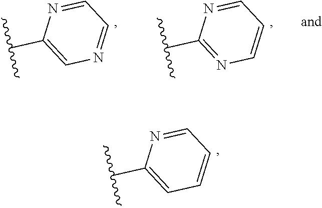 [SMILES: *c1cnccn1 ; *c1ncccn1 ; *c1ccccn1]

wherein Ring B is optionally substituted with 1 to 2 groups independently selected from
halogen
4- to 10-membered heterocyclyl (which may be optionally substituted with 1 to 3 groups independently selected from halogen, oxo, C₁-C₄ alkyl)
N(R^{x})₂, wherein R^{x} is selected from hydrogen, C₁-C₄ alkyl, C₃-C₆ cycloalkyl (which may be optionally substituted with a group selected from halogen, C₁-C₄ haloalkyl, and C₁-C₄ alkyl)
C₁-C₄ alkyl (optionally substituted with C₃-C₆ cycloalkyl (which may be further optionally substituted with a group selected from halogen, OH)).

In some embodiments, Ring B in the compound, tautomer, deuterated derivative, or salt of Formula II is selected from

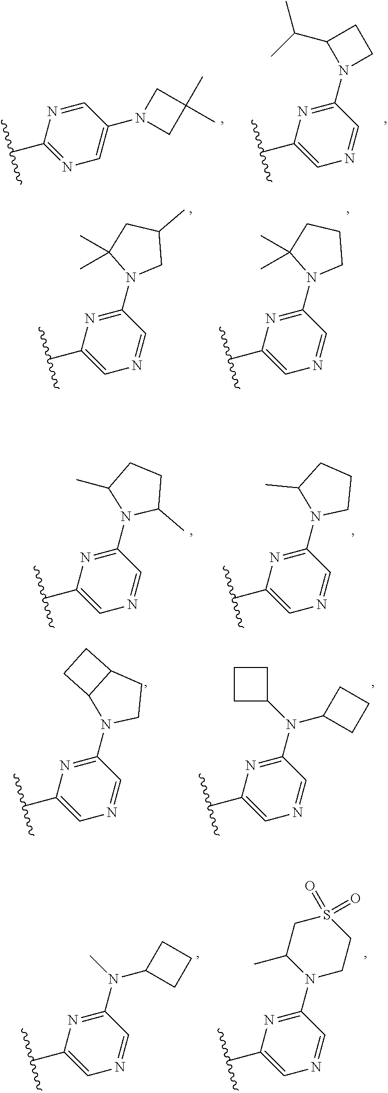

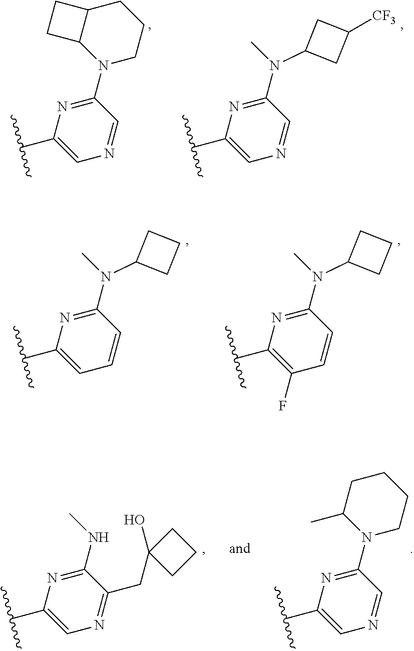 [SMILES: *c1cncc(N2CCCC3CCC32)n1 ; *c1cncc(N(C)C2CC(C(F)(F)F)C2)n1 ; *c1cccc(N(C)C2CCC2)n1 ; *c1nc(N(C)C2CCC2)ccc1F ; *c1cnc(CC2(O)CCC2)c(NC)n1 ; *c1cncc(N2CCCCC2C)n1]

In some embodiments, the compound of Formula II is selected from Compounds II-1 to II-38, tautomers thereof, deuterated derivatives of the compounds and tautomers, and pharmaceutically acceptable salts of any of the foregoing.

| Com- | |
|---|---|
| pound | |
| Num- | |
| ber | Structure |
| II-1 | 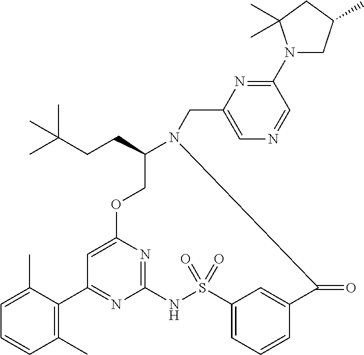 [SMILES: Cc1cccc(C)c1-c1cc2nc(n1)NS(=O)(=O)c1cccc(c1)C(=O)N(Cc1cncc(N3C[C@@H](C)CC3(C)C)n1)[C@H](CCC(C)(C)C)CO2] |
| II-2 | 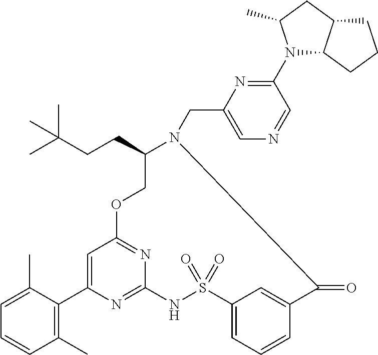 [SMILES: Cc1cccc(C)c1-c1cc2nc(n1)NS(=O)(=O)c1cccc(c1)C(=O)N(Cc1cncc(N3[C@H](C)C[C@@H]4CCC[C@@H]43)n1)[C@H](CCC(C)(C)C)CO2] |
| II-3 | 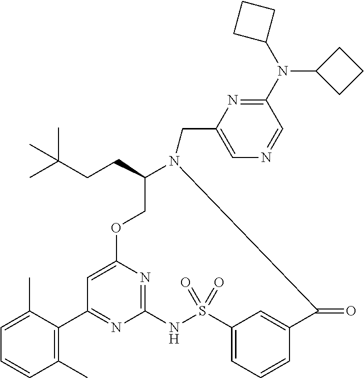 [SMILES: Cc1cccc(C)c1-c1cc2nc(n1)NS(=O)(=O)c1cccc(c1)C(=O)N(Cc1cncc(N(C3CCC3)C3CCC3)n1)[C@H](CCC(C)(C)C)CO2] |
| II-4 | 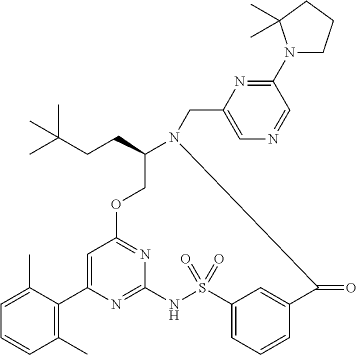 [SMILES: Cc1cccc(C)c1-c1cc2nc(n1)NS(=O)(=O)c1cccc(c1)C(=O)N(Cc1cncc(N3CCCC3(C)C)n1)[C@H](CCC(C)(C)C)CO2] |
| II-5 | 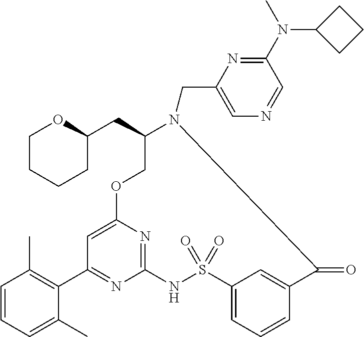 [SMILES: Cc1cccc(C)c1-c1cc2nc(n1)NS(=O)(=O)c1cccc(c1)C(=O)N(Cc1cncc(N(C)C3CCC3)n1)[C@H](C[C@H]1CCCCO1)CO2] |
| II-6 | 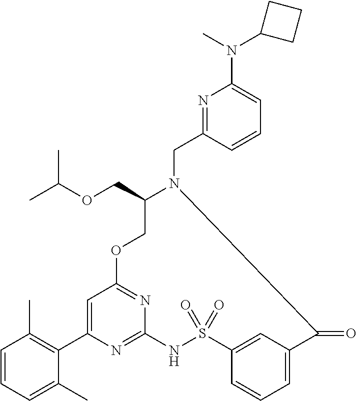 [SMILES: Cc1cccc(C)c1-c1cc2nc(n1)NS(=O)(=O)c1cccc(c1)C(=O)N(Cc1cccc(N(C)C3CCC3)n1)[C@H](COC(C)C)CO2] |
| II-7 | 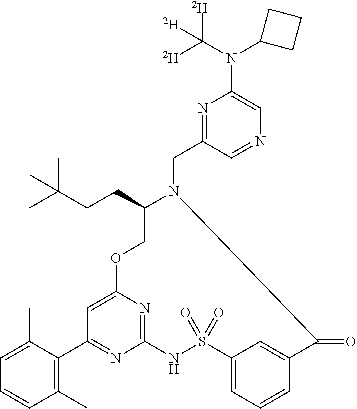 [SMILES: [2H]C([2H])([2H])N(c1cncc(CN2C(=O)c3cccc(c3)S(=O)(=O)Nc3nc(cc(-c4c(C)cccc4C)n3)OC[C@H]2CCC(C)(C)C)n1)C1CCC1] |
| II-8 | 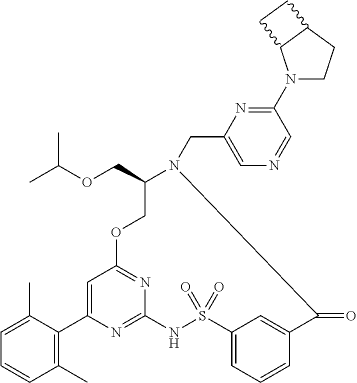 [SMILES: Cc1cccc(C)c1-c1cc2nc(n1)NS(=O)(=O)c1cccc(c1)C(=O)N(Cc1cncc(N3CCC4CCC43)n1)[C@H](COC(C)C)CO2] |
| II-9 | 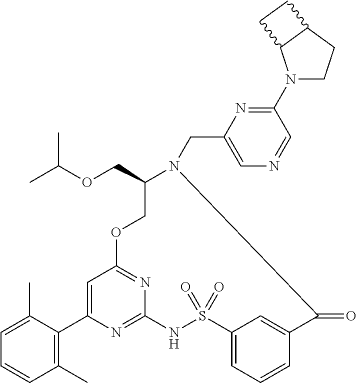 [SMILES: Cc1cccc(C)c1-c1cc2nc(n1)NS(=O)(=O)c1cccc(c1)C(=O)N(Cc1cncc(N3CCC4CCC43)n1)[C@H](COC(C)C)CO2] |
| II-10 | 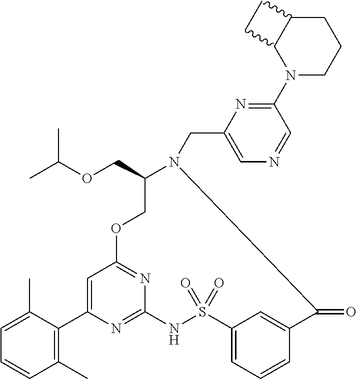 [SMILES: Cc1cccc(C)c1-c1cc2nc(n1)NS(=O)(=O)c1cccc(c1)C(=O)N(Cc1cncc(N3CCCC4CCC43)n1)[C@H](COC(C)C)CO2] |
| II-11 | 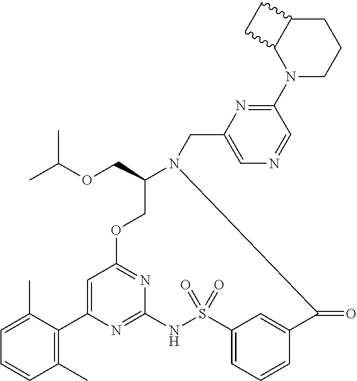 [SMILES: Cc1cccc(C)c1-c1cc2nc(n1)NS(=O)(=O)c1cccc(c1)C(=O)N(Cc1cncc(N3CCCC4CCC43)n1)[C@H](COC(C)C)CO2] |
| II-12 | 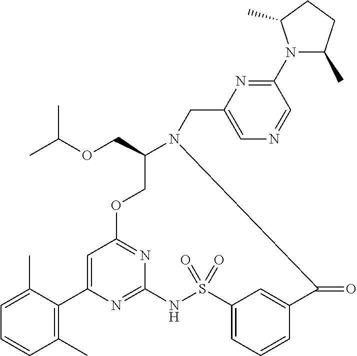 [SMILES: Cc1cccc(C)c1-c1cc2nc(n1)NS(=O)(=O)c1cccc(c1)C(=O)N(Cc1cncc(N3[C@H](C)CC[C@H]3C)n1)[C@H](COC(C)C)CO2] |
| II-13 | 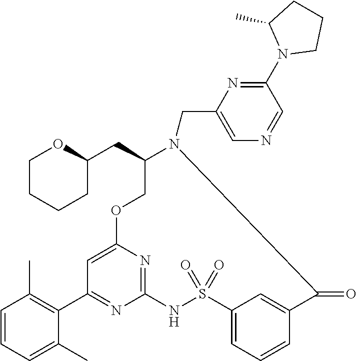 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@H]1CCCCO1)CO2] |
| II-14 | 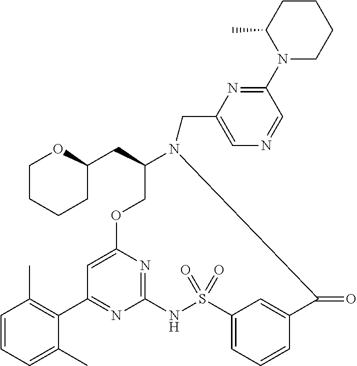 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@H]1CCCCO1)CO2] |
| II-15 | 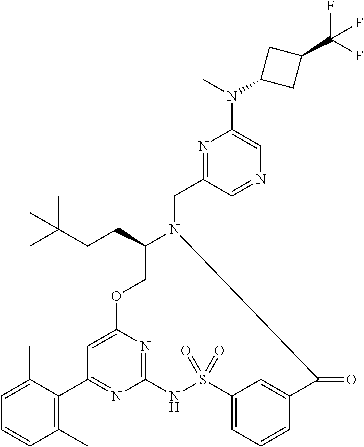 [SMILES: Cc1cccc(C)c1-c1cc2nc(n1)NS(=O)(=O)c1cccc(c1)C(=O)N(Cc1cncc(N(C)[C@H]3C[C@H](C(F)(F)F)C3)n1)[C@H](CCC(C)(C)C)CO2] |
| II-16 | 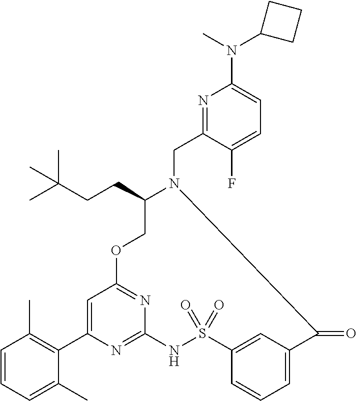 [SMILES: Cc1cccc(C)c1-c1cc2nc(n1)NS(=O)(=O)c1cccc(c1)C(=O)N(Cc1nc(N(C)C3CCC3)ccc1F)[C@H](CCC(C)(C)C)CO2] |
| II-17 | 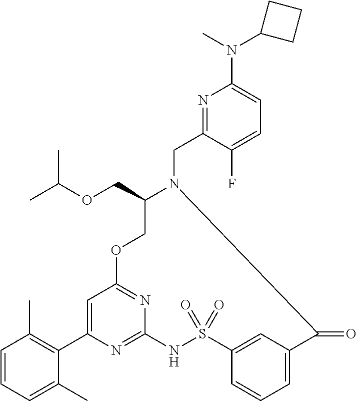 [SMILES: Cc1cccc(C)c1-c1cc2nc(n1)NS(=O)(=O)c1cccc(c1)C(=O)N(Cc1nc(N(C)C3CCC3)ccc1F)[C@H](COC(C)C)CO2] |
| II-18 | 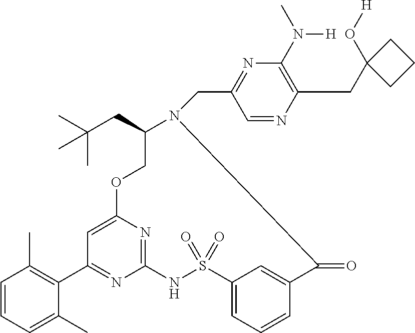 [SMILES: CNc1nc(CN2C(=O)c3cccc(c3)S(=O)(=O)Nc3nc(cc(-c4c(C)cccc4C)n3)OC[C@H]2CC(C)(C)C)cnc1CC1(O)CCC1] |
| II-19 | 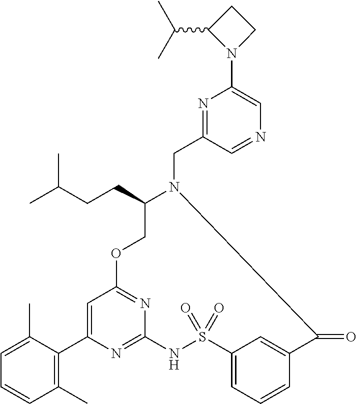 [SMILES: Cc1cccc(C)c1-c1cc2nc(n1)NS(=O)(=O)c1cccc(c1)C(=O)N(Cc1cncc(N3CCC3C(C)C)n1)[C@H](CCC(C)C)CO2] |
| II-20 | 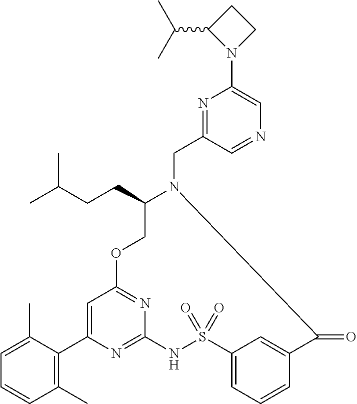 [SMILES: Cc1cccc(C)c1-c1cc2nc(n1)NS(=O)(=O)c1cccc(c1)C(=O)N(Cc1cncc(N3CCC3C(C)C)n1)[C@H](CCC(C)C)CO2] |
| II-21 | 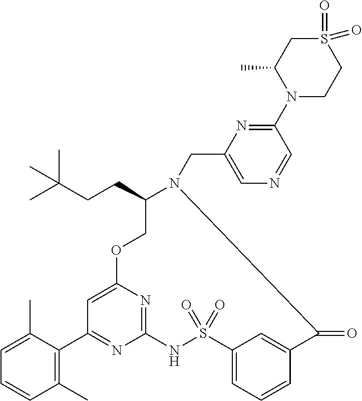 [SMILES: Cc1cccc(C)c1-c1cc2nc(n1)NS(=O)(=O)c1cccc(c1)C(=O)N(Cc1cncc(N3CCS(=O)(=O)C[C@H]3C)n1)[C@H](CCC(C)(C)C)CO2] |
| II-22 | 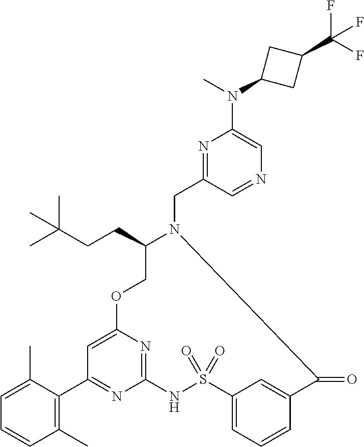 [SMILES: Cc1cccc(C)c1-c1cc2nc(n1)NS(=O)(=O)c1cccc(c1)C(=O)N(Cc1cncc(N(C)[C@H]3C[C@@H](C(F)(F)F)C3)n1)[C@H](CCC(C)(C)C)CO2] |
| II-23 | 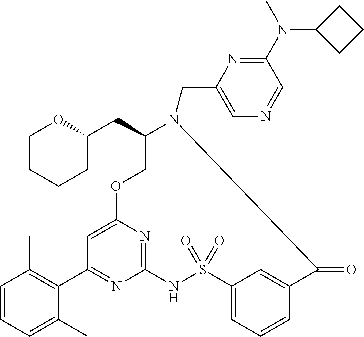 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCCO1)CO2] |
| II-24 | 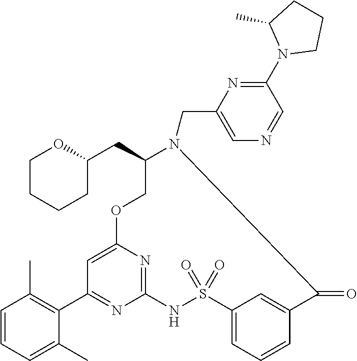 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@@H]1CCCCO1)CO2] |
| II-25 | 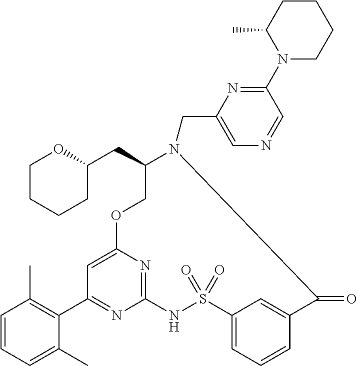 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@@H]1CCCCO1)CO2] |
| II-26 | 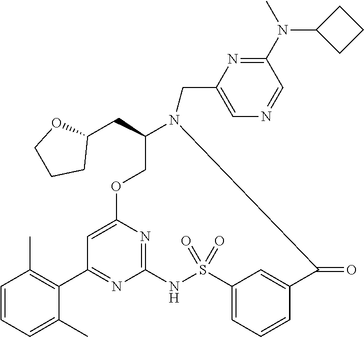 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCO1)CO2] |
| II-27 | 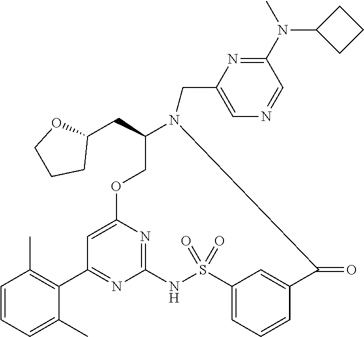 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCO1)CO2] |
| II-28 | 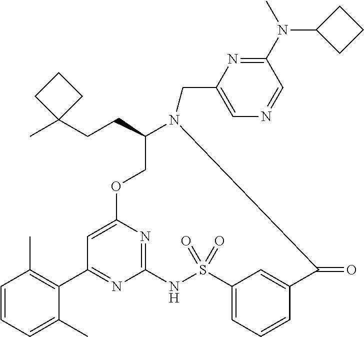 [SMILES: Cc1cccc(C)c1-c1cc2nc(n1)NS(=O)(=O)c1cccc(c1)C(=O)N(Cc1cncc(N(C)C3CCC3)n1)[C@H](CCC1(C)CCC1)CO2] |
| II-29 | 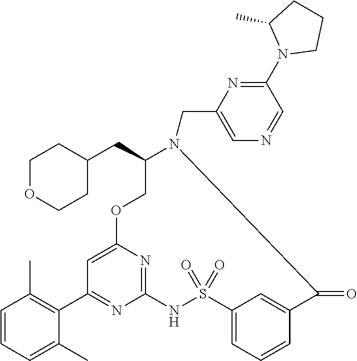 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CCOCC1)CO2] |
| II-30 | 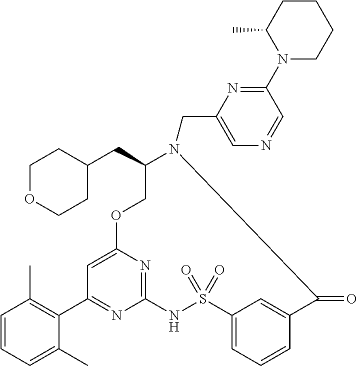 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CCOCC1)CO2] |
| II-31 | 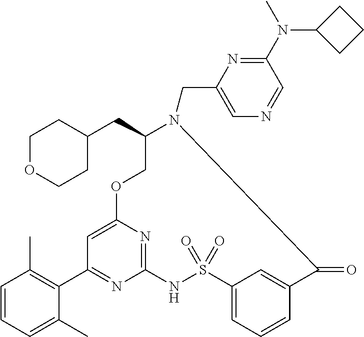 [SMILES: Cc1cccc(C)c1-c1cc2nc(n1)NS(=O)(=O)c1cccc(c1)C(=O)N(Cc1cncc(N(C)C3CCC3)n1)[C@H](CC1CCOCC1)CO2] |
| II-32 | 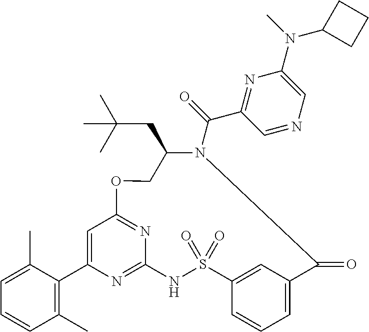 [SMILES: Cc1cccc(C)c1-c1cc2nc(n1)NS(=O)(=O)c1cccc(c1)C(=O)N(C(=O)c1cncc(N(C)C3CCC3)n1)[C@H](CC(C)(C)C)CO2] |
| II-33 | 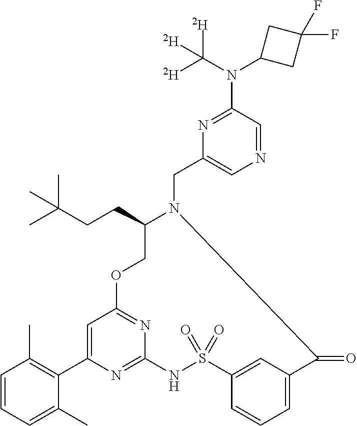 [SMILES: [2H]C([2H])([2H])N(c1cncc(CN2C(=O)c3cccc(c3)S(=O)(=O)Nc3nc(cc(-c4c(C)cccc4C)n3)OC[C@H]2CCC(C)(C)C)n1)C1CC(F)(F)C1] |
| II-34 | 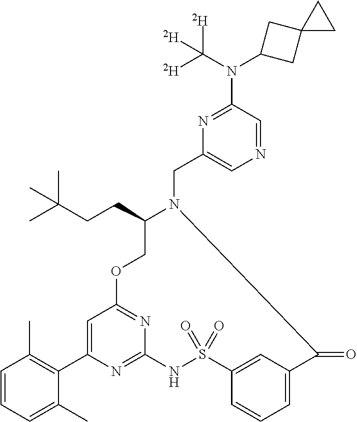 [SMILES: [2H]C([2H])([2H])N(c1cncc(CN2C(=O)c3cccc(c3)S(=O)(=O)Nc3nc(cc(-c4c(C)cccc4C)n3)OC[C@H]2CCC(C)(C)C)n1)C1CC2(CC2)C1] |
| II-35 | 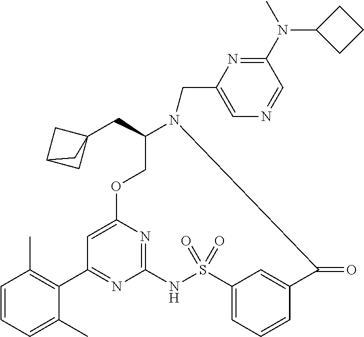 [SMILES: Cc1cccc(C)c1-c1cc2nc(n1)NS(=O)(=O)c1cccc(c1)C(=O)N(Cc1cncc(N(C)C3CCC3)n1)[C@H](CC13CC(C1)C3)CO2] |
| II-36 | 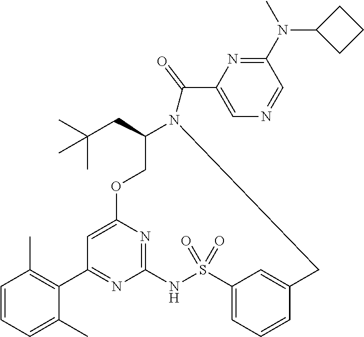 [SMILES: Cc1cccc(C)c1-c1cc2nc(n1)NS(=O)(=O)c1cccc(c1)CN(C(=O)c1cncc(N(C)C3CCC3)n1)[C@H](CC(C)(C)C)CO2] |
| II-37 | 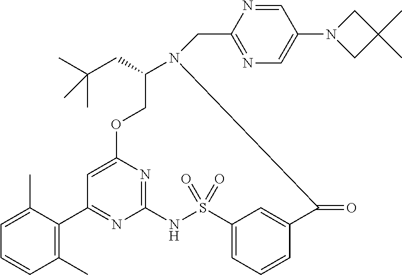 [SMILES: Cc1cccc(C)c1-c1cc2nc(n1)NS(=O)(=O)c1cccc(c1)C(=O)N(Cc1ncc(N3CC(C)(C)C3)cn1)[C@@H](CC(C)(C)C)CO2] |
| II-38 | 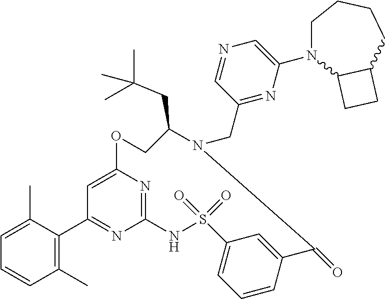 [SMILES: Cc1cccc(C)c1-c1cc2nc(n1)NS(=O)(=O)c1cccc(c1)C(=O)N(Cc1cncc(N3CCCCC4CCC43)n1)[C@H](CC(C)(C)C)CO2] |

In some embodiments, the disclosure provides compounds of Formula III:

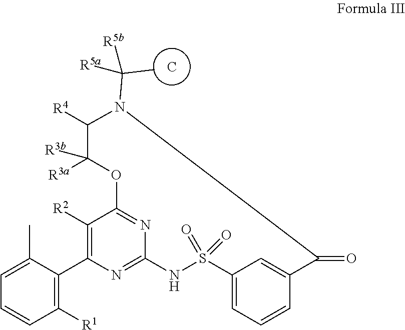

or a tautomer thereof, or a deuterated derivative of the compound or tautomer, or a pharmaceutically acceptable salt of any of the foregoing, wherein:
Ring C is selected from:

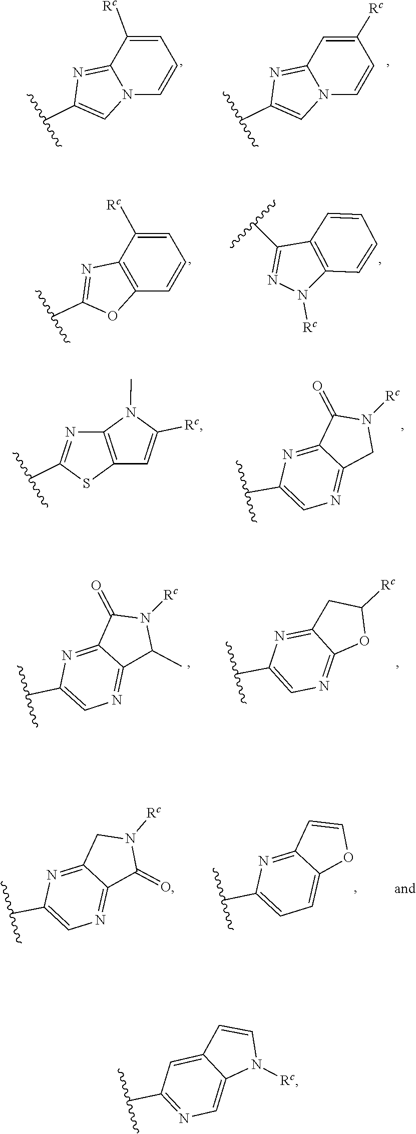

wherein
each R^{c} is independently selected from hydrogen, halogen, cyano, amino, C₁-C₄ alkyl (which may be optionally substituted with a group selected from —OH, halogen, and oxo), and C₃-C₆ alkenyl;
R¹ is selected from: C₃-C₆ cycloalkyl, C₁-C₆ alkoxy, and C₁-C₆ alkyl (which may be optionally substituted with a group selected from C₄-C₆ cycloalkyl, C₄-C₆ aryl, 4- to 6-membered heterocyclyl, and 4- to 6-membered heteroaryl);
R² is selected from: hydrogen, halogen, C₁-C₈ alkyl, haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);

In some embodiments, the compounds of Formula III are chosen from Compounds III-1 to III-25, tautomers thereof, deuterated derivatives of the compounds and tautomers, and pharmaceutically acceptable salts of any of the foregoing.

| Com- | |
|---|---|
| pound | |
| Num- | |
| ber | Structure |
| III-1 | 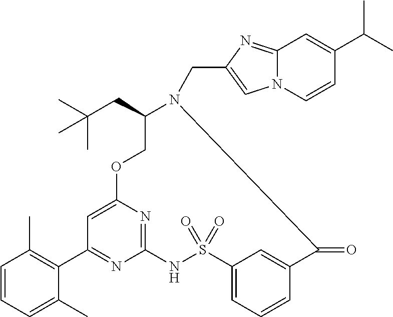 [SMILES: Cc1cccc(C)c1-c1cc2nc(n1)NS(=O)(=O)c1cccc(c1)C(=O)N(Cc1cn3ccc(C(C)C)cc3n1)[C@H](CC(C)(C)C)CO2] |
| III-2 | 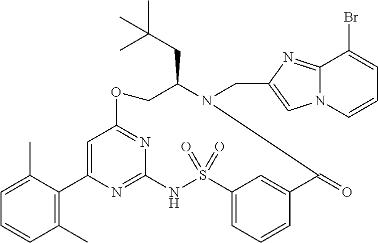 [SMILES: Cc1cccc(C)c1-c1cc2nc(n1)NS(=O)(=O)c1cccc(c1)C(=O)N(Cc1cn3cccc(Br)c3n1)[C@H](CC(C)(C)C)CO2] |
| III-3 | 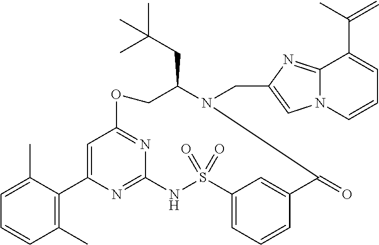 [SMILES: C=C(C)c1cccn2cc(CN3C(=O)c4cccc(c4)S(=O)(=O)Nc4nc(cc(-c5c(C)cccc5C)n4)OC[C@H]3CC(C)(C)C)nc12] |
| III-4 | 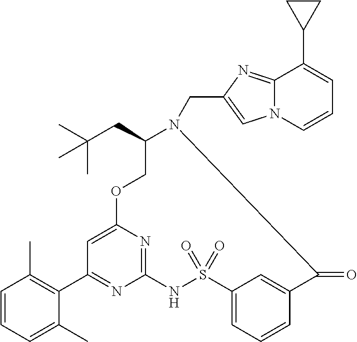 [SMILES: Cc1cccc(C)c1-c1cc2nc(n1)NS(=O)(=O)c1cccc(c1)C(=O)N(Cc1cn3cccc(C4CC4)c3n1)[C@H](CC(C)(C)C)CO2] |
| III-5 | 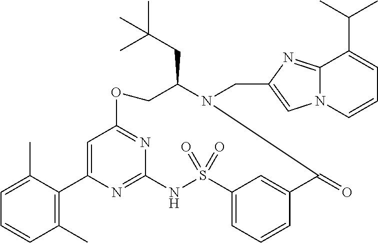 [SMILES: Cc1cccc(C)c1-c1cc2nc(n1)NS(=O)(=O)c1cccc(c1)C(=O)N(Cc1cn3cccc(C(C)C)c3n1)[C@H](CC(C)(C)C)CO2] |
| III-6 | 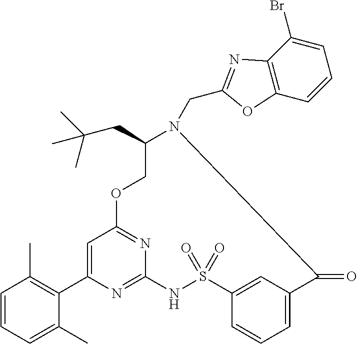 [SMILES: Cc1cccc(C)c1-c1cc2nc(n1)NS(=O)(=O)c1cccc(c1)C(=O)N(Cc1nc3c(Br)cccc3o1)[C@H](CC(C)(C)C)CO2] |
| III-7 | 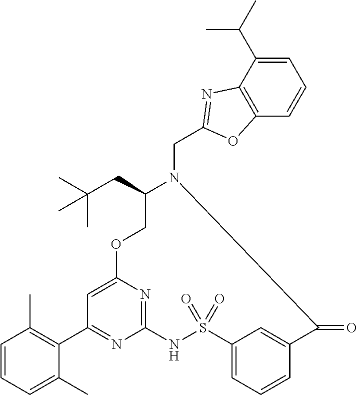 [SMILES: Cc1cccc(C)c1-c1cc2nc(n1)NS(=O)(=O)c1cccc(c1)C(=O)N(Cc1nc3c(C(C)C)cccc3o1)[C@H](CC(C)(C)C)CO2] |
| III-8 | 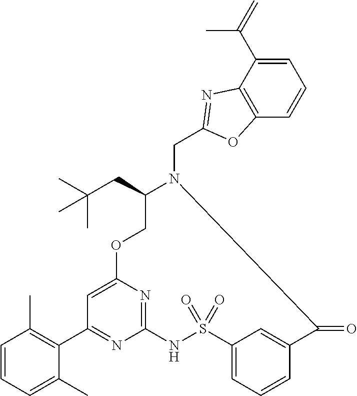 [SMILES: C=C(C)c1cccc2oc(CN3C(=O)c4cccc(c4)S(=O)(=O)Nc4nc(cc(-c5c(C)cccc5C)n4)OC[C@H]3CC(C)(C)C)nc12] |
| III-9 | 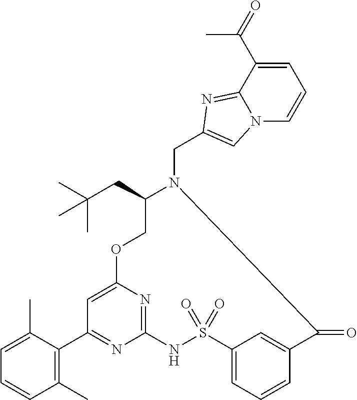 [SMILES: CC(=O)c1cccn2cc(CN3C(=O)c4cccc(c4)S(=O)(=O)Nc4nc(cc(-c5c(C)cccc5C)n4)OC[C@H]3CC(C)(C)C)nc12] |
| III-10 | 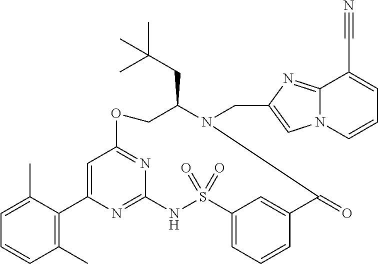 [SMILES: Cc1cccc(C)c1-c1cc2nc(n1)NS(=O)(=O)c1cccc(c1)C(=O)N(Cc1cn3cccc(C#N)c3n1)[C@H](CC(C)(C)C)CO2] |
| III-11 | 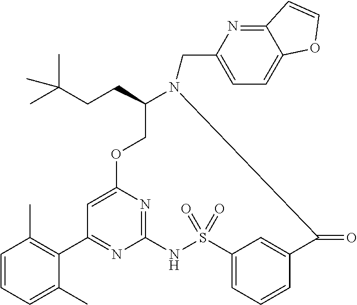 [SMILES: Cc1cccc(C)c1-c1cc2nc(n1)NS(=O)(=O)c1cccc(c1)C(=O)N(Cc1ccc3occc3n1)[C@H](CCC(C)(C)C)CO2] |
| III-12 | 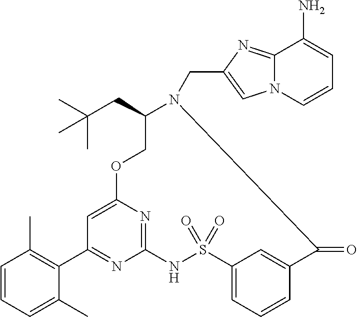 [SMILES: Cc1cccc(C)c1-c1cc2nc(n1)NS(=O)(=O)c1cccc(c1)C(=O)N(Cc1cn3cccc(N)c3n1)[C@H](CC(C)(C)C)CO2] |
| III-13 | 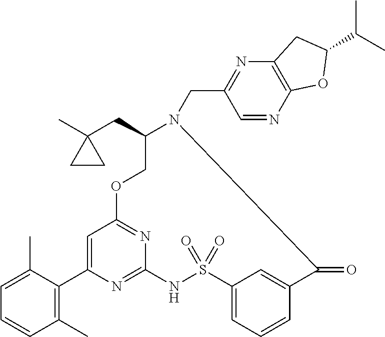 [SMILES: Cc1cccc(C)c1-c1cc2nc(n1)NS(=O)(=O)c1cccc(c1)C(=O)N(Cc1cnc3c(n1)C[C@H](C(C)C)O3)[C@H](CC1(C)CC1)CO2] |
| III-14 | 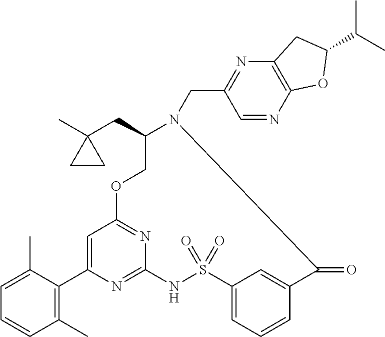 [SMILES: Cc1cccc(C)c1-c1cc2nc(n1)NS(=O)(=O)c1cccc(c1)C(=O)N(Cc1cnc3c(n1)C[C@H](C(C)C)O3)[C@H](CC1(C)CC1)CO2] |
| III-15 | 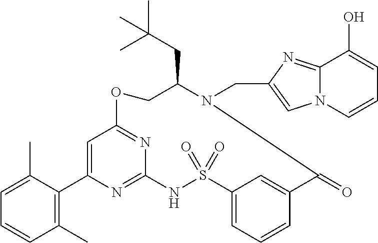 [SMILES: Cc1cccc(C)c1-c1cc2nc(n1)NS(=O)(=O)c1cccc(c1)C(=O)N(Cc1cn3cccc(O)c3n1)[C@H](CC(C)(C)C)CO2] |
| III-16 | 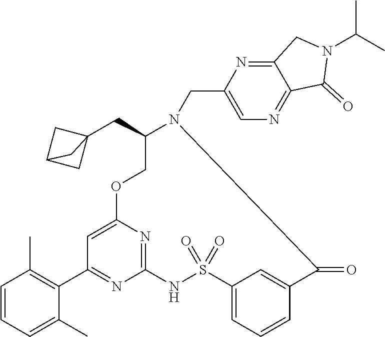 [SMILES: Cc1cccc(C)c1-c1cc2nc(n1)NS(=O)(=O)c1cccc(c1)C(=O)N(Cc1cnc3c(n1)CN(C(C)C)C3=O)[C@H](CC13CC(C1)C3)CO2] |
| III-17 | 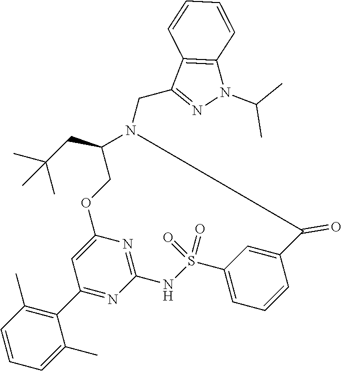 [SMILES: Cc1cccc(C)c1-c1cc2nc(n1)NS(=O)(=O)c1cccc(c1)C(=O)N(Cc1nn(C(C)C)c3ccccc13)[C@H](CC(C)(C)C)CO2] |
| III-18 | 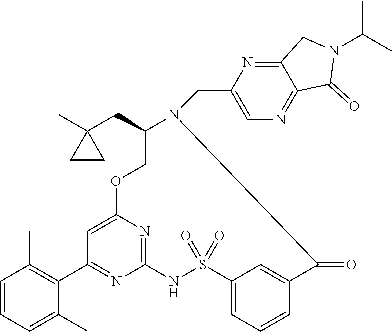 [SMILES: Cc1cccc(C)c1-c1cc2nc(n1)NS(=O)(=O)c1cccc(c1)C(=O)N(Cc1cnc3c(n1)CN(C(C)C)C3=O)[C@H](CC1(C)CC1)CO2] |
| III-19 | 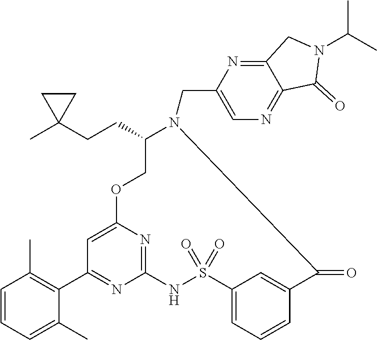 [SMILES: Cc1cccc(C)c1-c1cc2nc(n1)NS(=O)(=O)c1cccc(c1)C(=O)N(Cc1cnc3c(n1)CN(C(C)C)C3=O)[C@@H](CCC1(C)CC1)CO2] |
| III-20 | 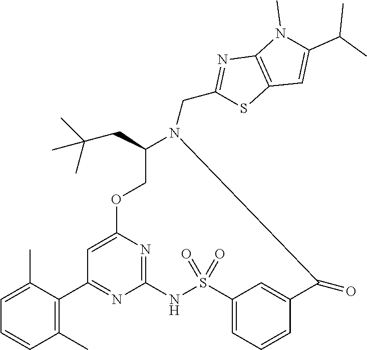 [SMILES: Cc1cccc(C)c1-c1cc2nc(n1)NS(=O)(=O)c1cccc(c1)C(=O)N(Cc1nc3c(cc(C(C)C)n3C)s1)[C@H](CC(C)(C)C)CO2] |
| III-21 | 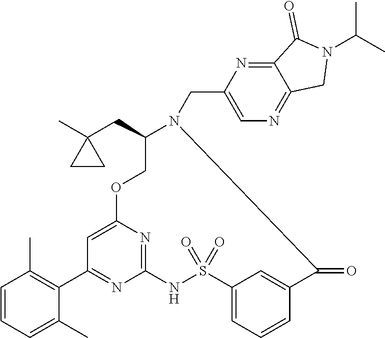 [SMILES: Cc1cccc(C)c1-c1cc2nc(n1)NS(=O)(=O)c1cccc(c1)C(=O)N(Cc1cnc3c(n1)C(=O)N(C(C)C)C3)[C@H](CC1(C)CC1)CO2] |
| III-22 | 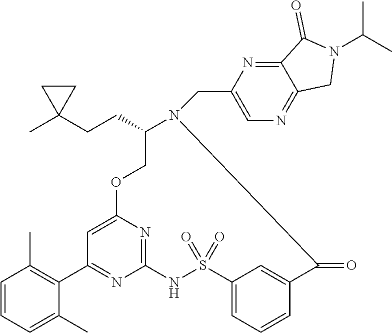 [SMILES: Cc1cccc(C)c1-c1cc2nc(n1)NS(=O)(=O)c1cccc(c1)C(=O)N(Cc1cnc3c(n1)C(=O)N(C(C)C)C3)[C@@H](CCC1(C)CC1)CO2] |
| III-23 | 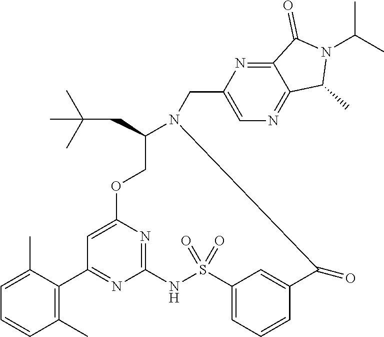 [SMILES: Cc1cccc(C)c1-c1cc2nc(n1)NS(=O)(=O)c1cccc(c1)C(=O)N(Cc1cnc3c(n1)C(=O)N(C(C)C)[C@@H]3C)[C@H](CC(C)(C)C)CO2] |
| III-24 | 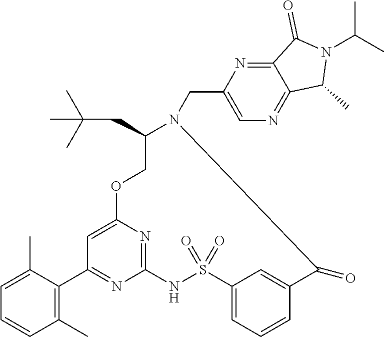 [SMILES: Cc1cccc(C)c1-c1cc2nc(n1)NS(=O)(=O)c1cccc(c1)C(=O)N(Cc1cnc3c(n1)C(=O)N(C(C)C)[C@@H]3C)[C@H](CC(C)(C)C)CO2] |
| III-25 | 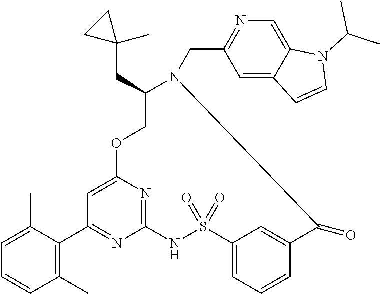 [SMILES: Cc1cccc(C)c1-c1cc2nc(n1)NS(=O)(=O)c1cccc(c1)C(=O)N(Cc1cc3ccn(C(C)C)c3cn1)[C@H](CC1(C)CC1)CO2] |

In some embodiments, the disclosure provides compounds of Formula IV:

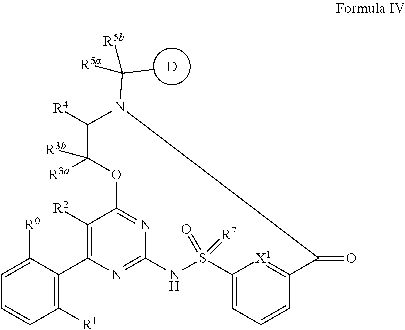

tautomers thereof, or a deuterated derivatives of the compounds and tautomers, and pharmaceutically acceptable salts of any of the foregoing, wherein:
Ring D is

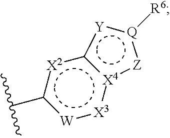

Q is selected from —C— and —N—;
W is selected from —CH—, —C(F)—, —C(CF₃)—, and —N—;
X¹, X², and X³ are each independently selected from —CH— and —N—;
X⁴ is selected from C and N;
Y is selected from —N—, —N(R^{y})—, —C(R^{y})—, and —O—, wherein
   R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CR^{z}—, —O—, —S—, —S(O)—, —S(O)₂—, —N—, and —N^{z}, wherein
   R^{z} is selected from hydrogen, halogen, and C₁-C₈ alkyl (which may be optionally substituted with C₁-C₈ alkoxy;
R⁰ is selected from C₁-C₂ alkyl;
R¹ is selected from: C₃-C₆ cycloalkyl, C₁-C₈ alkoxy, and C₁-C₈ alkyl (which may be optionally substituted with a group selected from C₁-C₈ alkoxy, C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₈ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   —OH
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   —OC₃-C₇ cycloalkyl (which may be optionally substituted with 1 to 2 groups selected from C₁-C₈ alkyl, C₁-C₈ alkoxy, halogen, and C₁-C₈ haloalkyl);
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl, and phenyl; or which may be optionally substituted with 1 to 3 halogen atoms);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁶ is selected from hydrogen, cyano, halogen, 4- to 6-membered heterocyclyl, 5- to 6-membered heteroaryl (which may be optionally substituted with 1 to 2 groups selected from C₁-C₈ alkyl), C₃-C₅ cycloalkyl (which may be optionally substituted with a group selected from C₁-C₈ alkyl, C₁-C₈ haloalkyl, and halogen), phenyl, and C₁-C₈ alkyl (which may be optionally substituted with 1 to 2 groups independently selected from C₁-C₈ alkoxy, C₁-C₈ haloalkyl, halogen, oxo, —OH, —NH₂, and —SO₂CH₃); and
R⁷ is selected from O, and NR, wherein
   R is selected from hydrogen and C₁-C₈ alkyl;
      with the proviso that the compound of Formula IV is not selected from:

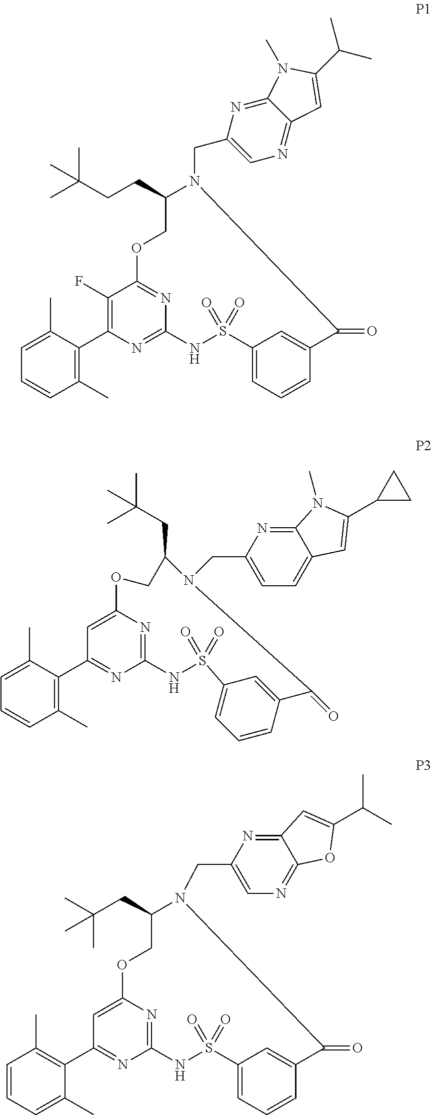 [SMILES: Cc1cccc(C)c1-c1nc2nc(c1F)OC[C@@H](CCC(C)(C)C)N(Cc1cnc3cc(C(C)C)n(C)c3n1)C(=O)c1cccc(c1)S(=O)(=O)N2 ; Cc1cccc(C)c1-c1cc2nc(n1)NS(=O)(=O)c1cccc(c1)C(=O)N(Cc1ccc3cc(C4CC4)n(C)c3n1)[C@H](CC(C)(C)C)CO2 ; Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2]

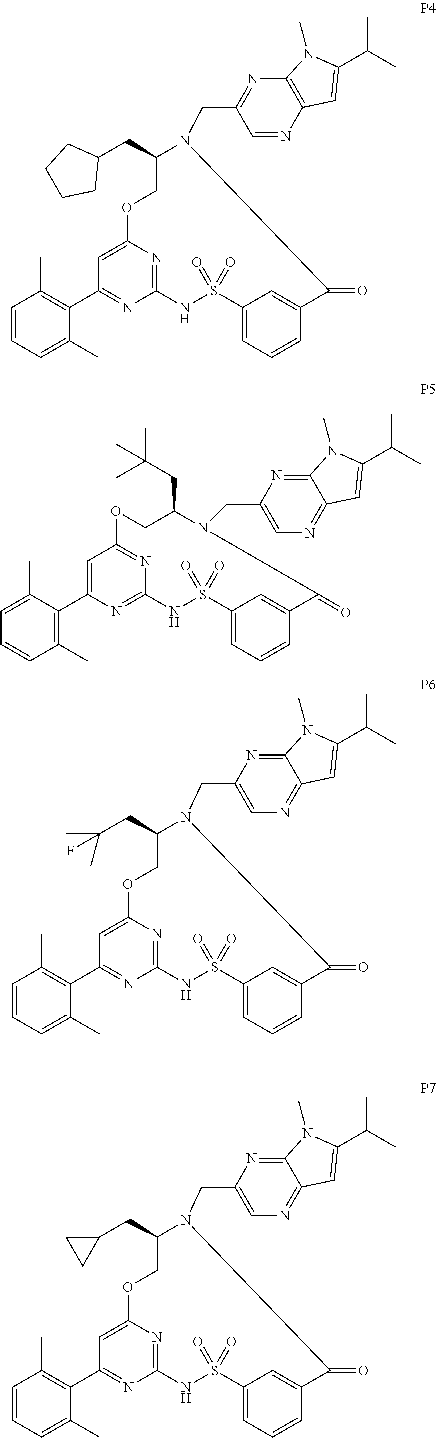 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)F)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1CC1)CO2]

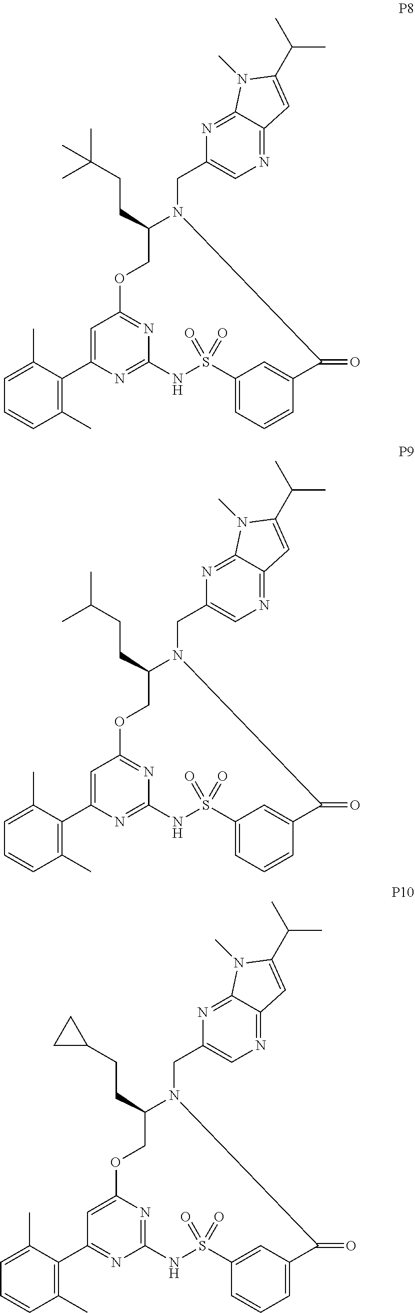

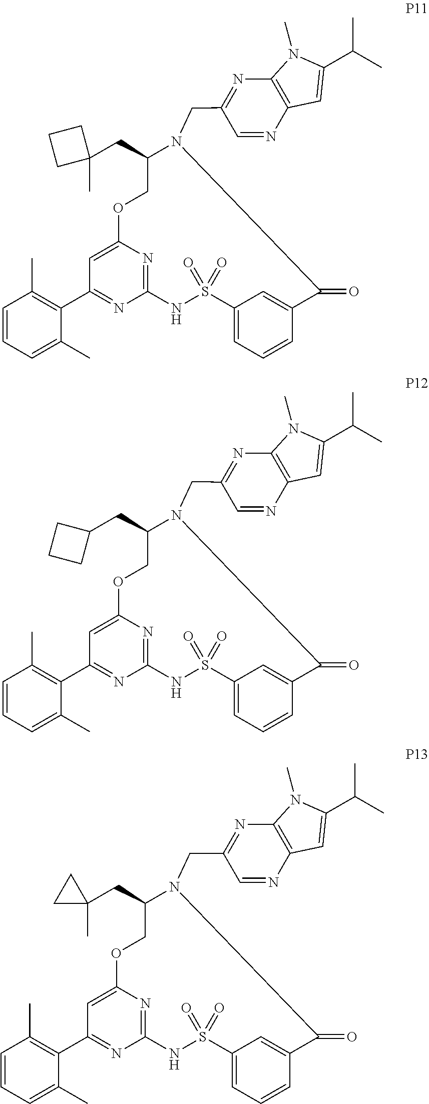

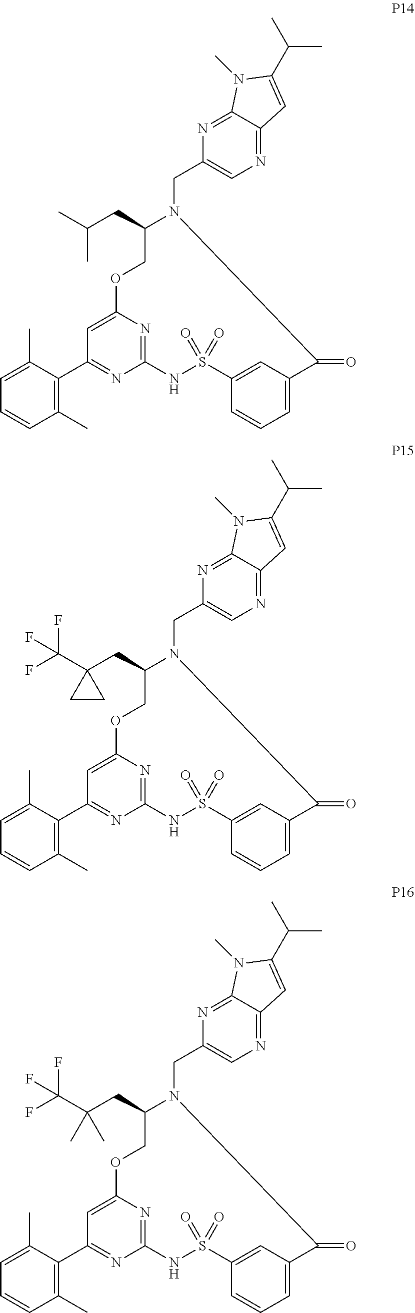

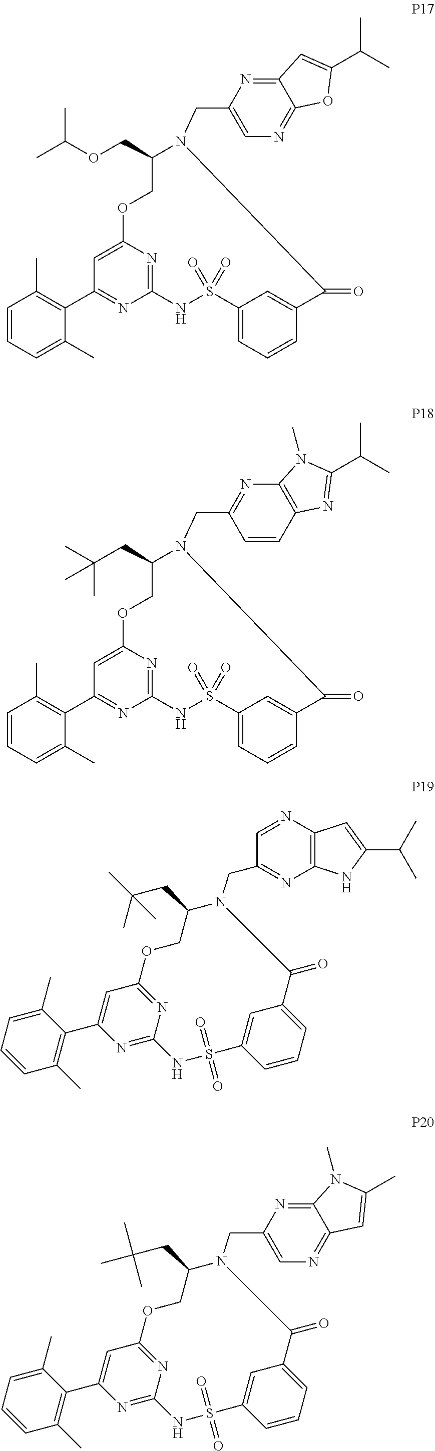 [SMILES: Cc1cccc(C)c1-c1cc2nc(n1)NS(=O)(=O)c1cccc(c1)C(=O)N(Cc1cnc3oc(C(C)C)cc3n1)[C@H](COC(C)C)CO2 ; Cc1cccc(C)c1-c1cc2nc(n1)NS(=O)(=O)c1cccc(c1)C(=O)N(Cc1ccc3nc(C(C)C)n(C)c3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)[nH]c3n1)[C@H](CC(C)(C)C)CO2 ; Cc1cccc(C)c1-c1cc2nc(n1)NS(=O)(=O)c1cccc(c1)C(=O)N(Cc1cnc3cc(C)n(C)c3n1)[C@H](CC(C)(C)C)CO2]

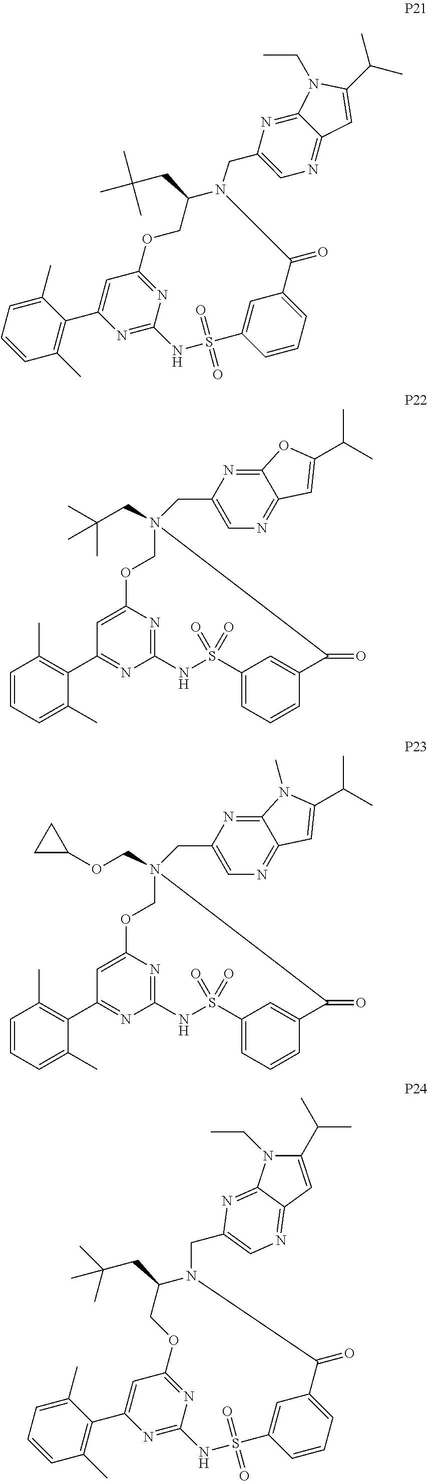

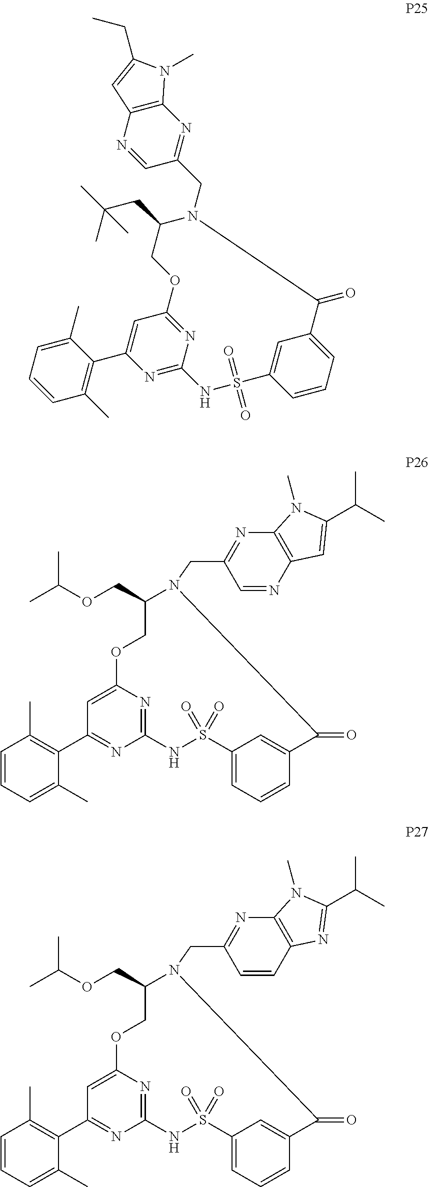 [SMILES: CCc1cc2ncc(CN3C(=O)c4cccc(c4)S(=O)(=O)Nc4nc(cc(-c5c(C)cccc5C)n4)OC[C@H]3CC(C)(C)C)nc2n1C ; Cc1cccc(C)c1-c1cc2nc(n1)NS(=O)(=O)c1cccc(c1)C(=O)N(Cc1cnc3cc(C(C)C)n(C)c3n1)[C@H](COC(C)C)CO2 ; Cc1cccc(C)c1-c1cc2nc(n1)NS(=O)(=O)c1cccc(c1)C(=O)N(Cc1ccc3nc(C(C)C)n(C)c3n1)[C@H](COC(C)C)CO2]

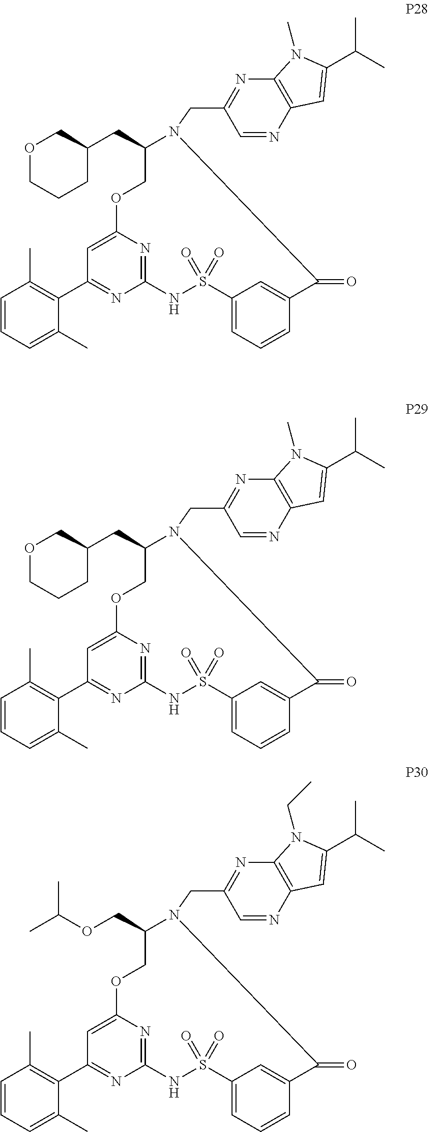 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCOC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCOC1)CO2 ; CCn1c(C(C)C)cc2ncc(CN3C(=O)c4cccc(c4)S(=O)(=O)Nc4nc(cc(-c5c(C)cccc5C)n4)OC[C@H]3COC(C)C)nc21]

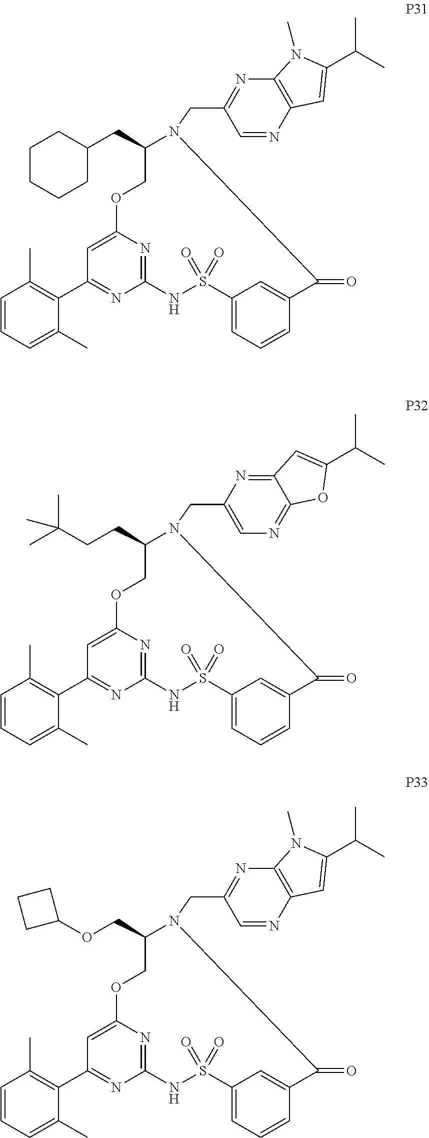 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CCC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](COC1CCC1)CO2]

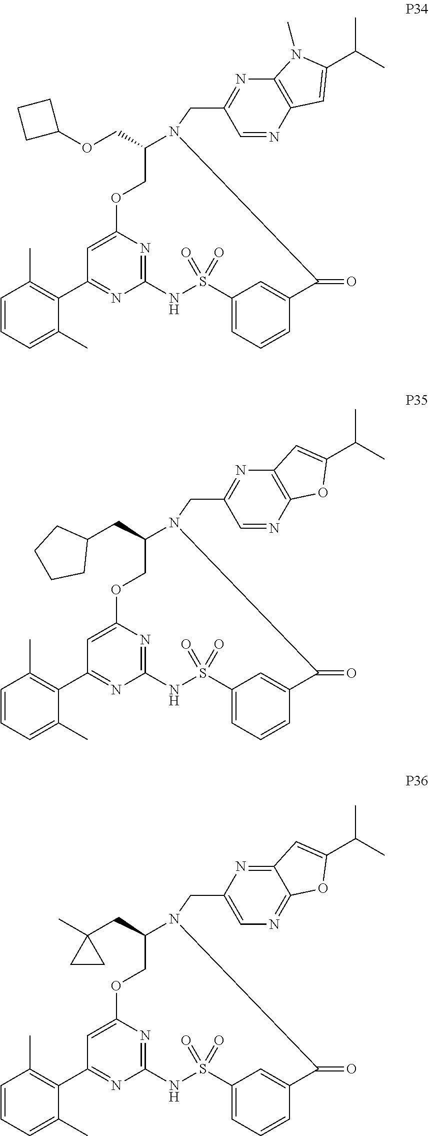 [SMILES: Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1CCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2]

and tautomers, deuterated derivatives, and pharmaceutically acceptable salts thereof.

In some embodiments, the compound of Formula IV is selected from Compounds IV-1 to IV-106, tautomers thereof, deuterated derivatives of the compounds and tautomers, and pharmaceutically acceptable salts of any of the foregoing.

| Cmpd | |
|---|---|
| Num- | |
| ber | Structure |
| IV-1 | 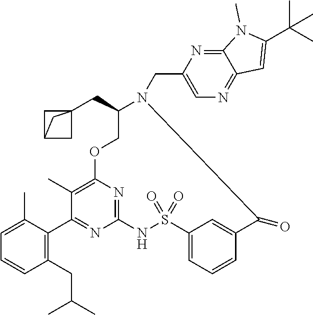 [SMILES: Cc1cccc(CC(C)C)c1-c1nc2nc(c1C)OC[C@@H](CC13CC(C1)C3)N(Cc1cnc3cc(C(C)(C)C)n(C)c3n1)C(=O)c1cccc(c1)S(=O)(=O)N2] |
| IV-2 | 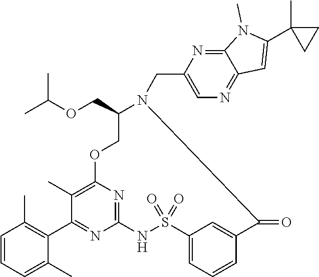 [SMILES: Cc1cccc(C)c1-c1nc2nc(c1C)OC[C@@H](COC(C)C)N(Cc1cnc3cc(C4(C)CC4)n(C)c3n1)C(=O)c1cccc(c1)S(=O)(=O)N2] |
| IV-3 | 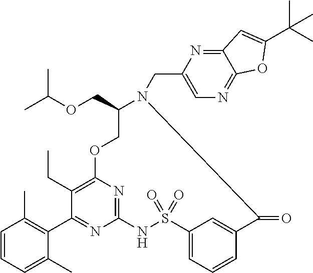 [SMILES: CCc1c2nc(nc1-c1c(C)cccc1C)NS(=O)(=O)c1cccc(c1)C(=O)N(Cc1cnc3oc(C(C)(C)C)cc3n1)[C@H](COC(C)C)CO2] |
| IV-4 | 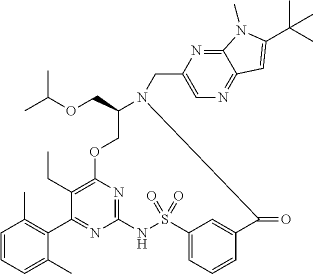 [SMILES: CCc1c2nc(nc1-c1c(C)cccc1C)NS(=O)(=O)c1cccc(c1)C(=O)N(Cc1cnc3cc(C(C)(C)C)n(C)c3n1)[C@H](COC(C)C)CO2] |
| IV-5 | 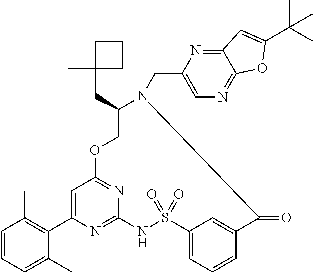 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1(C)CCC1)CO2] |
| IV-6 | 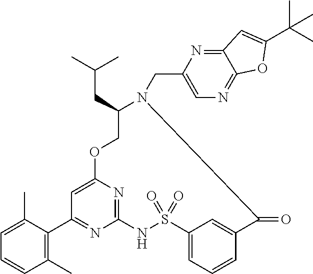 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC(C)C)CO2] |
| IV-7 | 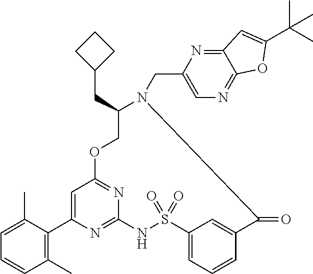 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCC1)CO2] |
| IV-8 | 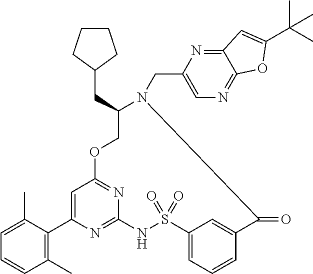 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCCC1)CO2] |
| IV-9 | 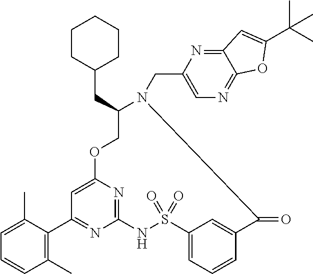 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCCCC1)CO2] |
| IV-10 | 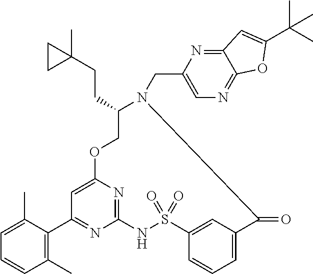 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CCC1(C)CC1)CO2] |
| IV-11 | 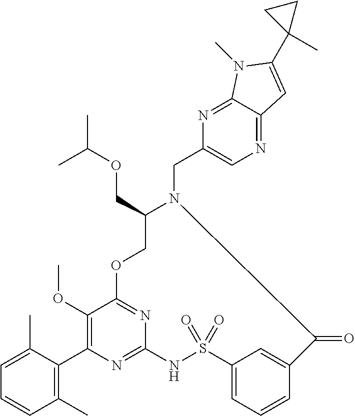 [SMILES: COc1c2nc(nc1-c1c(C)cccc1C)NS(=O)(=O)c1cccc(c1)C(=O)N(Cc1cnc3cc(C4(C)CC4)n(C)c3n1)[C@H](COC(C)C)CO2] |
| IV-12 | 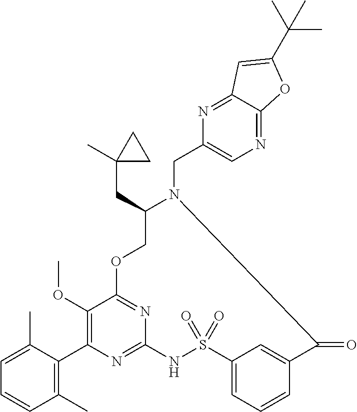 [SMILES: COc1c2nc(nc1-c1c(C)cccc1C)NS(=O)(=O)c1cccc(c1)C(=O)N(Cc1cnc3oc(C(C)(C)C)cc3n1)[C@H](CC1(C)CC1)CO2] |
| IV-13 | 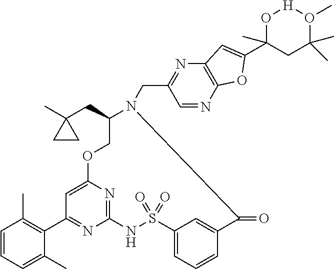 |
| IV-14 | 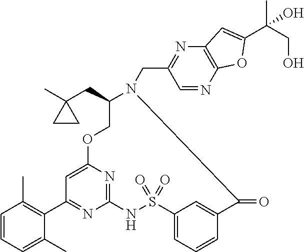 [SMILES: Cc1cccc(C)c1-c1cc2nc(n1)NS(=O)(=O)c1cccc(c1)C(=O)N(Cc1cnc3oc([C@](C)(O)CO)cc3n1)[C@H](CC1(C)CC1)CO2] |
| IV-15 | 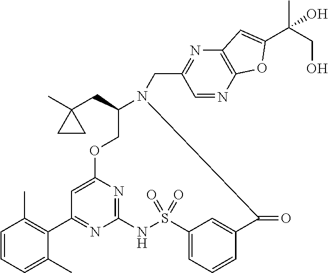 [SMILES: Cc1cccc(C)c1-c1cc2nc(n1)NS(=O)(=O)c1cccc(c1)C(=O)N(Cc1cnc3oc([C@](C)(O)CO)cc3n1)[C@H](CC1(C)CC1)CO2] |
| IV-16 | 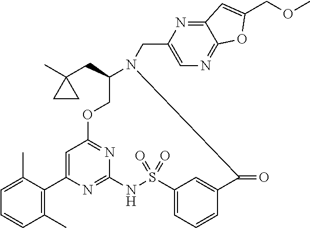 [SMILES: COCc1cc2nc(CN3C(=O)c4cccc(c4)S(=O)(=O)Nc4nc(cc(-c5c(C)cccc5C)n4)OC[C@H]3CC3(C)CC3)cnc2o1] |
| IV-17 | 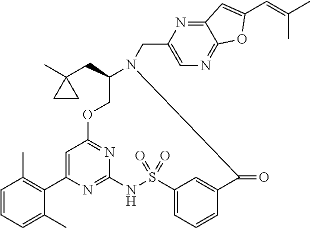 [SMILES: CC(C)=Cc1cc2nc(CN3C(=O)c4cccc(c4)S(=O)(=O)Nc4nc(cc(-c5c(C)cccc5C)n4)OC[C@H]3CC3(C)CC3)cnc2o1] |
| IV-18 | 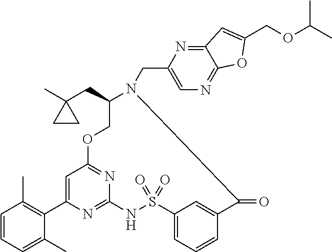 [SMILES: Cc1cccc(C)c1-c1cc2nc(n1)NS(=O)(=O)c1cccc(c1)C(=O)N(Cc1cnc3oc(COC(C)C)cc3n1)[C@H](CC1(C)CC1)CO2] |
| IV-19 | 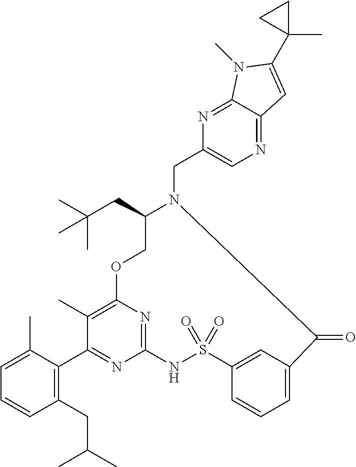 [SMILES: Cc1cccc(CC(C)C)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] |
| IV-20 | 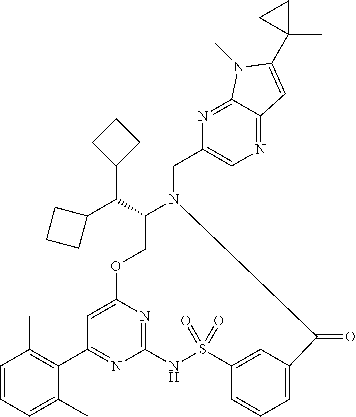 [SMILES: Cc1cccc(C)c1-c1cc2nc(n1)NS(=O)(=O)c1cccc(c1)C(=O)N(Cc1cnc3cc(C4(C)CC4)n(C)c3n1)[C@@H](C(C1CCC1)C1CCC1)CO2] |
| IV-21 | 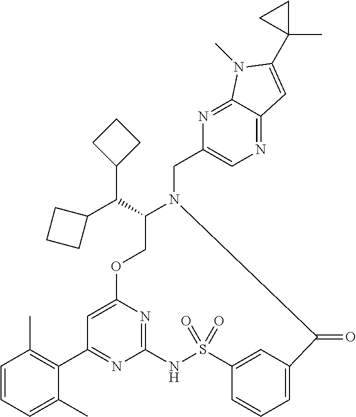 [SMILES: Cc1cccc(C)c1-c1cc2nc(n1)NS(=O)(=O)c1cccc(c1)C(=O)N(Cc1cnc3cc(C4(C)CC4)n(C)c3n1)[C@@H](C(C1CCC1)C1CCC1)CO2] |
| IV-22 | 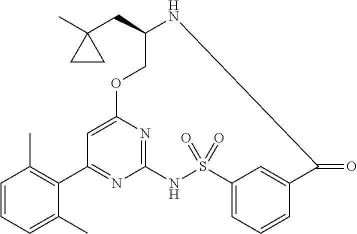 [SMILES: Cc1cccc(C)c1-c1cc2nc(n1)NS(=O)(=O)c1cccc(c1)C(=O)N[C@H](CC1(C)CC1)CO2] |
| IV-23 | 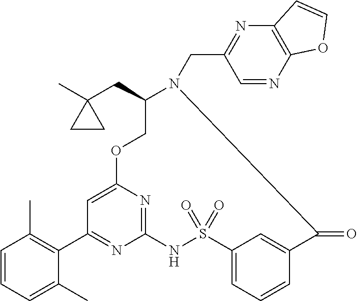 [SMILES: Cc1cccc(C)c1-c1cc2nc(n1)NS(=O)(=O)c1cccc(c1)C(=O)N(Cc1cnc3occc3n1)[C@H](CC1(C)CC1)CO2] |
| IV-24 | 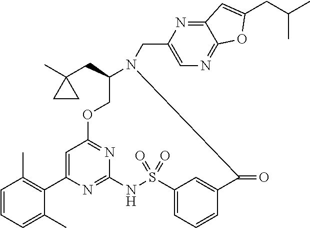 [SMILES: Cc1cccc(C)c1-c1cc2nc(n1)NS(=O)(=O)c1cccc(c1)C(=O)N(Cc1cnc3oc(CC(C)C)cc3n1)[C@H](CC1(C)CC1)CO2] |
| IV-25 | 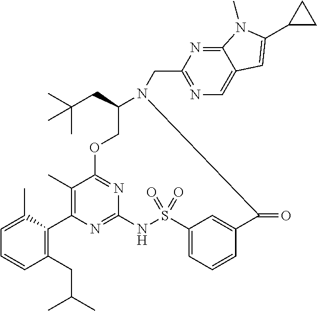 |
| IV-26 | 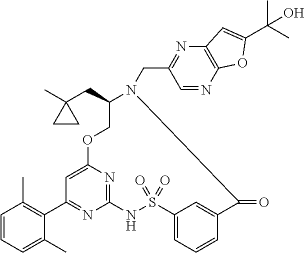 [SMILES: Cc1cccc(C)c1-c1cc2nc(n1)NS(=O)(=O)c1cccc(c1)C(=O)N(Cc1cnc3oc(C(C)(C)O)cc3n1)[C@H](CC1(C)CC1)CO2] |
| IV-27 | 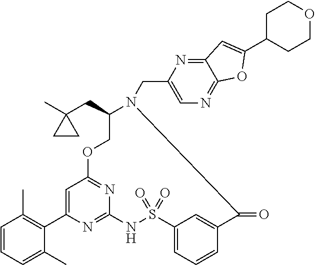 [SMILES: Cc1cccc(C)c1-c1cc2nc(n1)NS(=O)(=O)c1cccc(c1)C(=O)N(Cc1cnc3oc(C4CCOCC4)cc3n1)[C@H](CC1(C)CC1)CO2] |
| IV-28 | 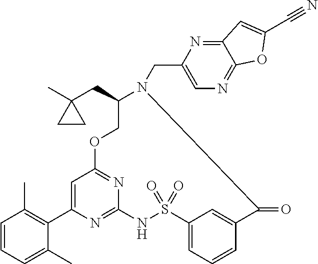 [SMILES: Cc1cccc(C)c1-c1cc2nc(n1)NS(=O)(=O)c1cccc(c1)C(=O)N(Cc1cnc3oc(C#N)cc3n1)[C@H](CC1(C)CC1)CO2] |
| IV-29 | 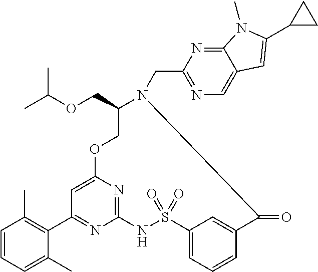 [SMILES: Cc1cccc(C)c1-c1cc2nc(n1)NS(=O)(=O)c1cccc(c1)C(=O)N(Cc1ncc3cc(C4CC4)n(C)c3n1)[C@H](COC(C)C)CO2] |
| IV-30 | 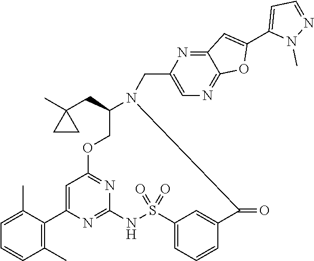 [SMILES: Cc1cccc(C)c1-c1cc2nc(n1)NS(=O)(=O)c1cccc(c1)C(=O)N(Cc1cnc3oc(-c4ccnn4C)cc3n1)[C@H](CC1(C)CC1)CO2] |
| IV-31 | 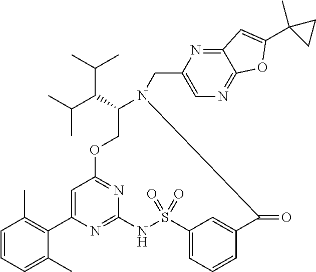 [SMILES: Cc1cccc(C)c1-c1cc2nc(n1)NS(=O)(=O)c1cccc(c1)C(=O)N(Cc1cnc3oc(C4(C)CC4)cc3n1)[C@@H](C(C(C)C)C(C)C)CO2] |
| IV-32 | 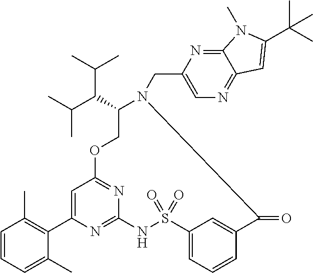 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](C(C(C)C)C(C)C)CO2] |
| IV-33 | 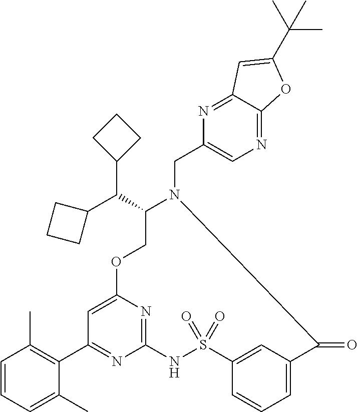 [SMILES: Cc1cccc(C)c1-c1cc2nc(n1)NS(=O)(=O)c1cccc(c1)C(=O)N(Cc1cnc3oc(C(C)(C)C)cc3n1)[C@@H](C(C1CCC1)C1CCC1)CO2] |
| IV-34 | 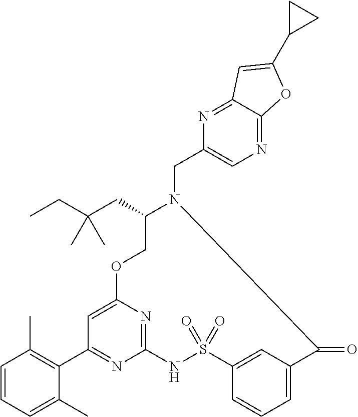 [SMILES: CCC(C)(C)C[C@H]1COc2cc(-c3c(C)cccc3C)nc(n2)NS(=O)(=O)c2cccc(c2)C(=O)N1Cc1cnc2oc(C3CC3)cc2n1] |
| IV-35 | 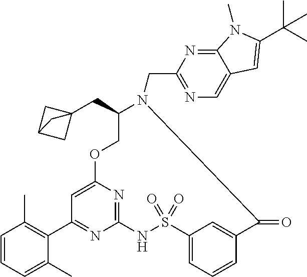 [SMILES: Cc1cccc(C)c1-c1cc2nc(n1)NS(=O)(=O)c1cccc(c1)C(=O)N(Cc1ncc3cc(C(C)(C)C)n(C)c3n1)[C@H](CC13CC(C1)C3)CO2] |
| IV-36 | 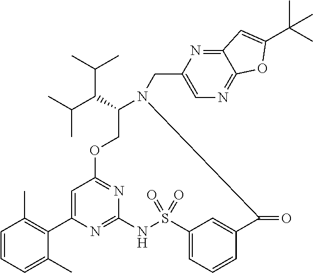 [SMILES: Cc1cccc(C)c1-c1cc2nc(n1)NS(=O)(=O)c1cccc(c1)C(=O)N(Cc1cnc3oc(C(C)(C)C)cc3n1)[C@@H](C(C(C)C)C(C)C)CO2] |
| IV-37 | 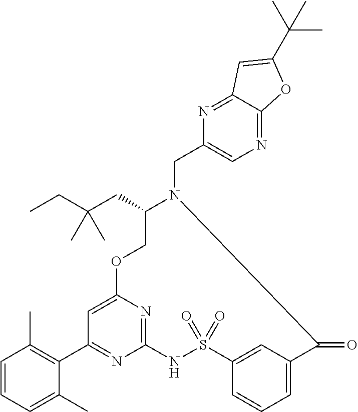 [SMILES: CCC(C)(C)C[C@H]1COc2cc(-c3c(C)cccc3C)nc(n2)NS(=O)(=O)c2cccc(c2)C(=O)N1Cc1cnc2oc(C(C)(C)C)cc2n1] |
| IV-38 | 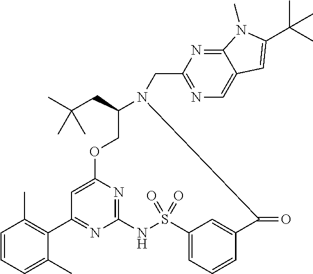 [SMILES: Cc1cccc(C)c1-c1cc2nc(n1)NS(=O)(=O)c1cccc(c1)C(=O)N(Cc1ncc3cc(C(C)(C)C)n(C)c3n1)[C@H](CC(C)(C)C)CO2] |
| IV-39 | 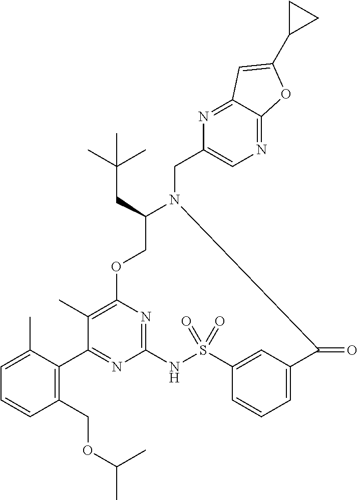 [SMILES: Cc1cccc(COC(C)C)c1-c1nc2nc(c1C)OC[C@@H](CC(C)(C)C)N(Cc1cnc3oc(C4CC4)cc3n1)C(=O)c1cccc(c1)S(=O)(=O)N2] |
| IV-40 | 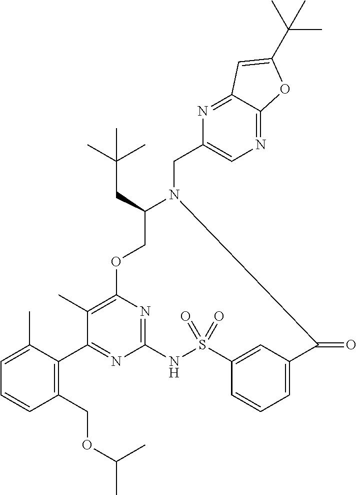 [SMILES: Cc1cccc(COC(C)C)c1-c1nc2nc(c1C)OC[C@@H](CC(C)(C)C)N(Cc1cnc3oc(C(C)(C)C)cc3n1)C(=O)c1cccc(c1)S(=O)(=O)N2] |
| IV-41 | 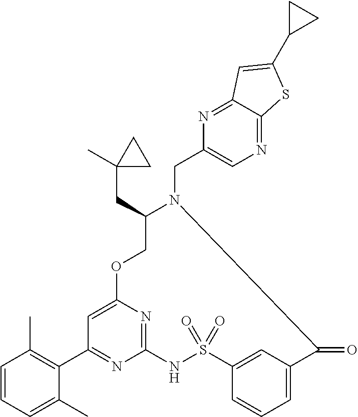 [SMILES: Cc1cccc(C)c1-c1cc2nc(n1)NS(=O)(=O)c1cccc(c1)C(=O)N(Cc1cnc3sc(C4CC4)cc3n1)[C@H](CC1(C)CC1)CO2] |
| IV-42 | 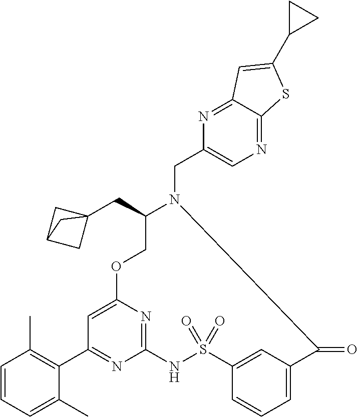 [SMILES: Cc1cccc(C)c1-c1cc2nc(n1)NS(=O)(=O)c1cccc(c1)C(=O)N(Cc1cnc3sc(C4CC4)cc3n1)[C@H](CC13CC(C1)C3)CO2] |
| IV-43 | 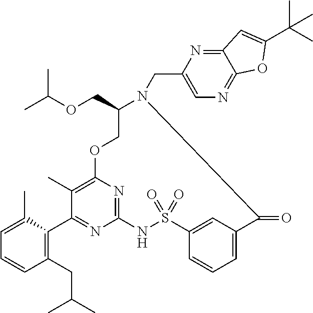 |
| IV-44 | 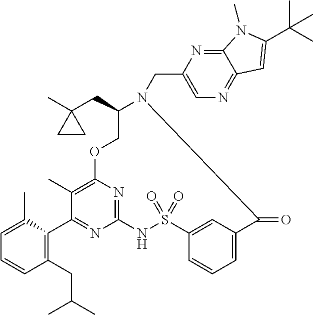 |
| IV-45 | 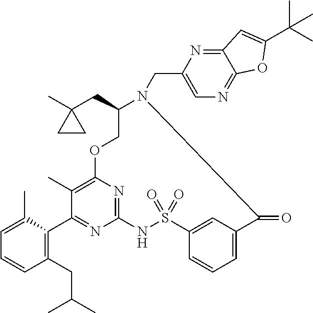 |
| IV-46 | 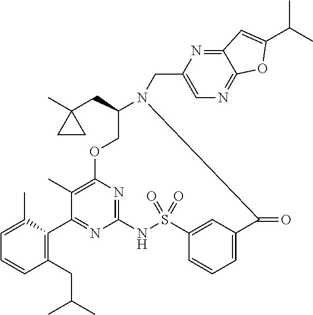 |
| IV-47 | 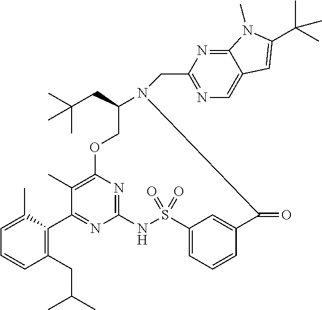 |
| IV-48 | 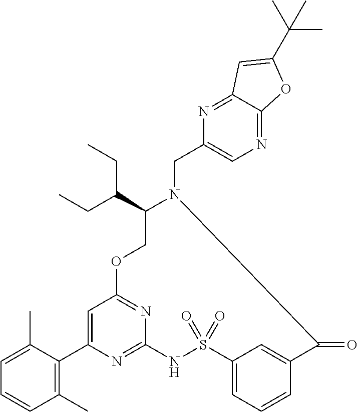 [SMILES: CCC(CC)[C@@H]1COc2cc(-c3c(C)cccc3C)nc(n2)NS(=O)(=O)c2cccc(c2)C(=O)N1Cc1cnc2oc(C(C)(C)C)cc2n1] |
| IV-49 | 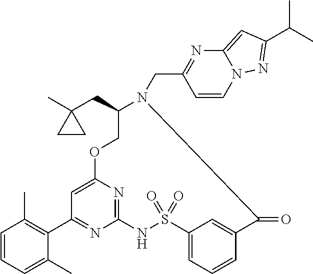 [SMILES: Cc1cccc(C)c1-c1cc2nc(n1)NS(=O)(=O)c1cccc(c1)C(=O)N(Cc1ccn3nc(C(C)C)cc3n1)[C@H](CC1(C)CC1)CO2] |
| IV-50 | 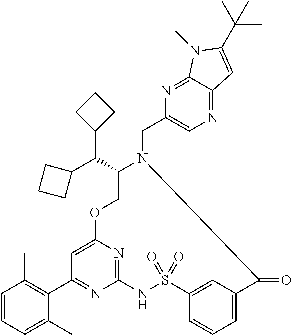 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](C(C1CCC1)C1CCC1)CO2] |
| IV-50 | 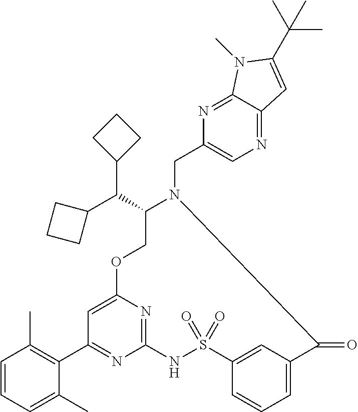 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](C(C1CCC1)C1CCC1)CO2] |
| IV-51 | 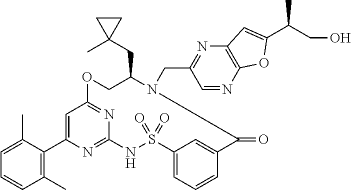 [SMILES: Cc1cccc(C)c1-c1cc2nc(n1)NS(=O)(=O)c1cccc(c1)C(=O)N(Cc1cnc3oc([C@@H](C)CO)cc3n1)[C@H](CC1(C)CC1)CO2] |
| IV-52 | 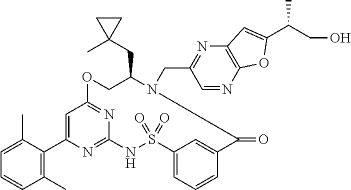 [SMILES: Cc1cccc(C)c1-c1cc2nc(n1)NS(=O)(=O)c1cccc(c1)C(=O)N(Cc1cnc3oc([C@H](C)CO)cc3n1)[C@H](CC1(C)CC1)CO2] |
| IV-53 | 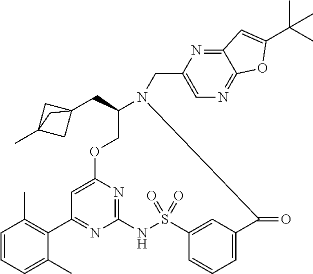 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC13CC(C)(C1)C3)CO2] |
| IV-54 | 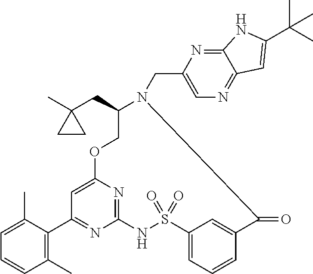 [SMILES: Cc1cccc(C)c1-c1cc2nc(n1)NS(=O)(=O)c1cccc(c1)C(=O)N(Cc1cnc3cc(C(C)(C)C)[nH]c3n1)[C@H](CC1(C)CC1)CO2] |
| IV-55 | 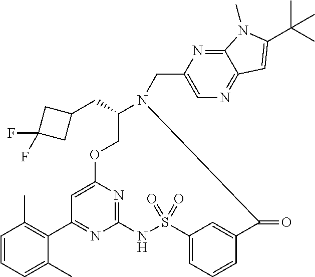 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](CC1CC(F)(F)C1)CO2] |
| IV-56 | 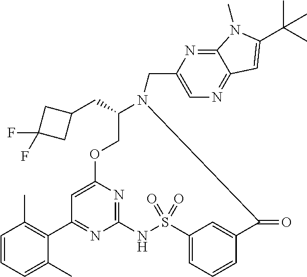 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](CC1CC(F)(F)C1)CO2] |
| IV-57 | 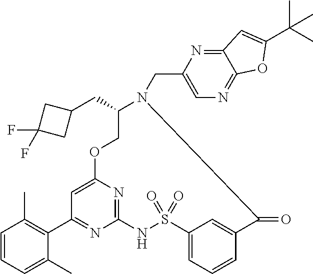 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CC1CC(F)(F)C1)CO2] |
| IV-58 | 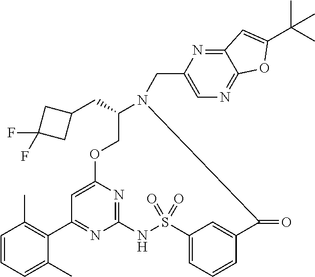 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CC1CC(F)(F)C1)CO2] |
| IV-59 | 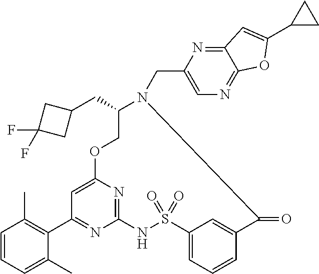 [SMILES: Cc1cccc(C)c1-c1cc2nc(n1)NS(=O)(=O)c1cccc(c1)C(=O)N(Cc1cnc3oc(C4CC4)cc3n1)[C@@H](CC1CC(F)(F)C1)CO2] |
| IV-60 | 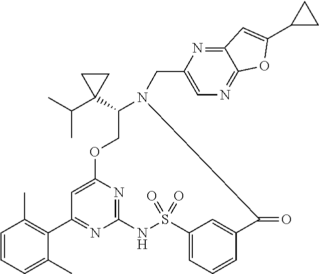 [SMILES: Cc1cccc(C)c1-c1cc2nc(n1)NS(=O)(=O)c1cccc(c1)C(=O)N(Cc1cnc3oc(C4CC4)cc3n1)[C@@H](C1(C(C)C)CC1)CO2] |
| IV-61 | 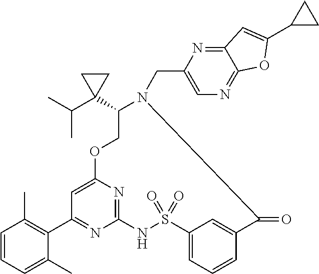 [SMILES: Cc1cccc(C)c1-c1cc2nc(n1)NS(=O)(=O)c1cccc(c1)C(=O)N(Cc1cnc3oc(C4CC4)cc3n1)[C@@H](C1(C(C)C)CC1)CO2] |
| IV-62 | 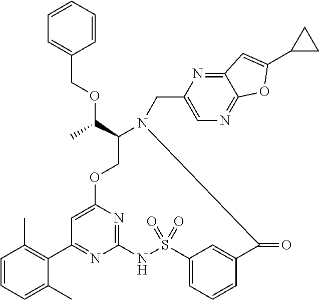 [SMILES: Cc1cccc(C)c1-c1cc2nc(n1)NS(=O)(=O)c1cccc(c1)C(=O)N(Cc1cnc3oc(C4CC4)cc3n1)[C@H]([C@H](C)OCc1ccccc1)CO2] |
| IV-63 | 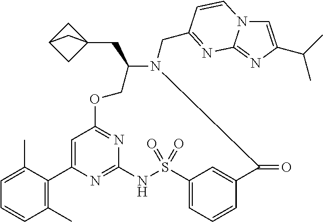 [SMILES: Cc1cccc(C)c1-c1cc2nc(n1)NS(=O)(=O)c1cccc(c1)C(=O)N(Cc1ccn3cc(C(C)C)nc3n1)[C@H](CC13CC(C1)C3)CO2] |
| IV-64 | 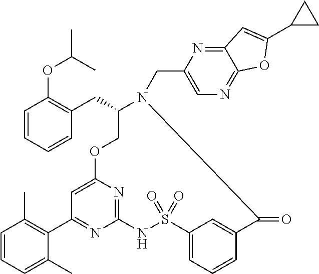 [SMILES: Cc1cccc(C)c1-c1cc2nc(n1)NS(=O)(=O)c1cccc(c1)C(=O)N(Cc1cnc3oc(C4CC4)cc3n1)[C@@H](Cc1ccccc1OC(C)C)CO2] |
| IV-65 | 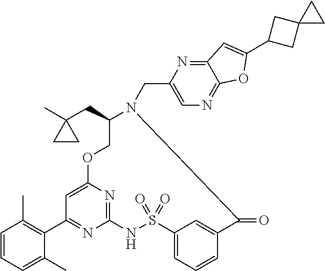 [SMILES: Cc1cccc(C)c1-c1cc2nc(n1)NS(=O)(=O)c1cccc(c1)C(=O)N(Cc1cnc3oc(C4CC5(CC5)C4)cc3n1)[C@H](CC1(C)CC1)CO2] |
| IV-66 | 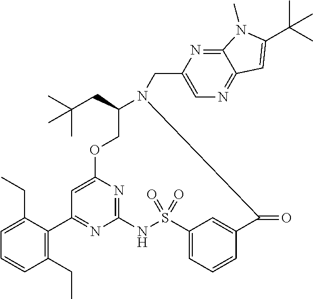 [SMILES: CCc1cccc(CC)c1-c1cc2nc(n1)NS(=O)(=O)c1cccc(c1)C(=O)N(Cc1cnc3cc(C(C)(C)C)n(C)c3n1)[C@H](CC(C)(C)C)CO2] |
| IV-67 | 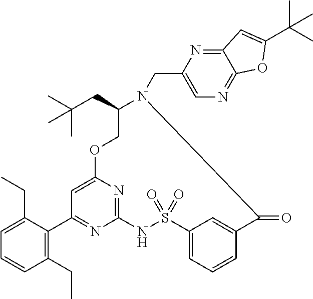 [SMILES: CCc1cccc(CC)c1-c1cc2nc(n1)NS(=O)(=O)c1cccc(c1)C(=O)N(Cc1cnc3oc(C(C)(C)C)cc3n1)[C@H](CC(C)(C)C)CO2] |
| IV-68 | 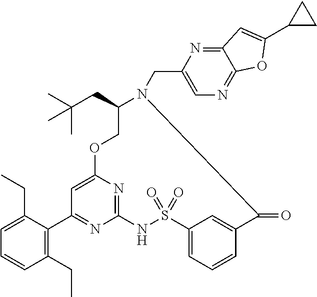 [SMILES: CCc1cccc(CC)c1-c1cc2nc(n1)NS(=O)(=O)c1cccc(c1)C(=O)N(Cc1cnc3oc(C4CC4)cc3n1)[C@H](CC(C)(C)C)CO2] |
| IV-69 | 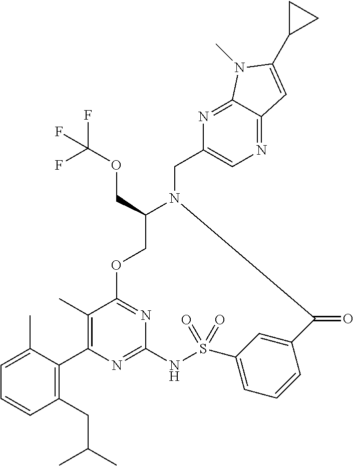 [SMILES: Cc1cccc(CC(C)C)c1-c1nc2nc(c1C)OC[C@@H](COC(F)(F)F)N(Cc1cnc3cc(C4CC4)n(C)c3n1)C(=O)c1cccc(c1)S(=O)(=O)N2] |
| IV-70 | 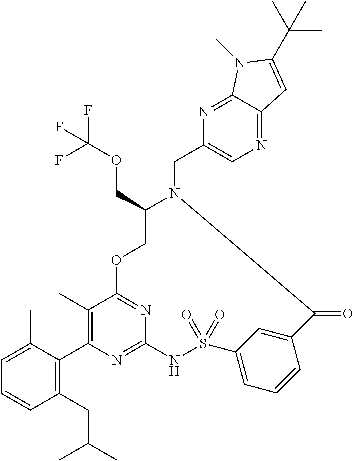 [SMILES: Cc1cccc(CC(C)C)c1-c1nc2nc(c1C)OC[C@@H](COC(F)(F)F)N(Cc1cnc3cc(C(C)(C)C)n(C)c3n1)C(=O)c1cccc(c1)S(=O)(=O)N2] |
| IV-71 | 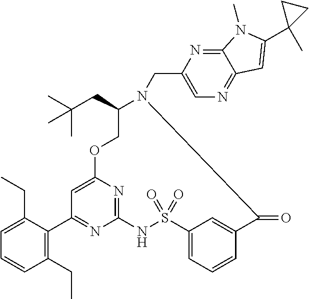 [SMILES: CCc1cccc(CC)c1-c1cc2nc(n1)NS(=O)(=O)c1cccc(c1)C(=O)N(Cc1cnc3cc(C4(C)CC4)n(C)c3n1)[C@H](CC(C)(C)C)CO2] |
| IV-72 | 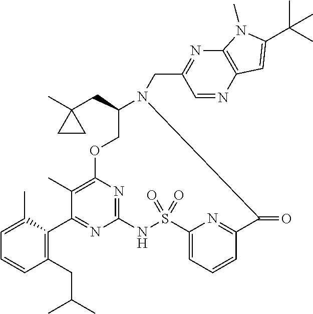 |
| IV-73 | 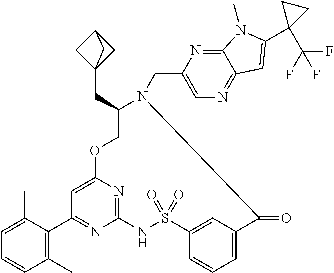 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](CC13CC(C1)C3)CO2] |
| IV-74 | 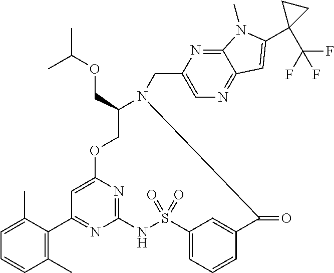 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](COC(C)C)CO2] |
| IV-75 | 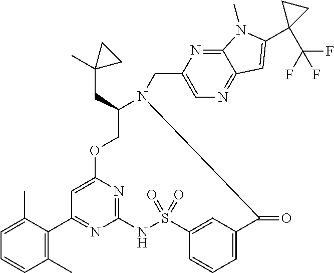 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](CC1(C)CC1)CO2] |
| IV-76 | 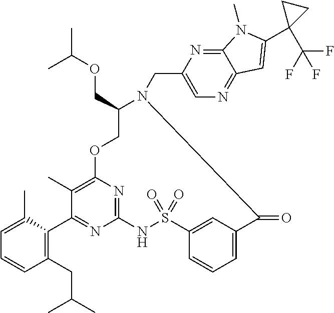 |
| IV-77 | 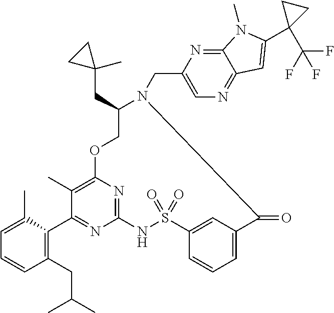 |
| IV-78 | 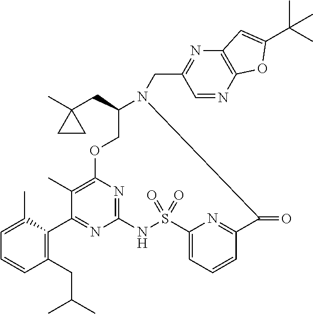 |
| IV-79 | 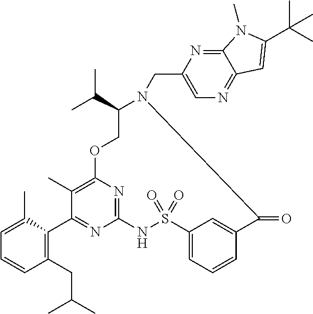 |
| IV-80 | 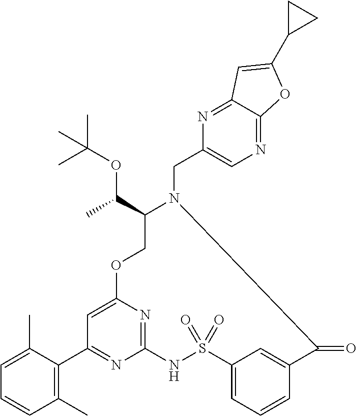 [SMILES: Cc1cccc(C)c1-c1cc2nc(n1)NS(=O)(=O)c1cccc(c1)C(=O)N(Cc1cnc3oc(C4CC4)cc3n1)[C@H]([C@H](C)OC(C)(C)C)CO2] |
| IV-81 | 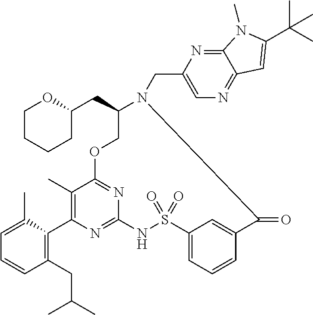 |
| IV-82 | 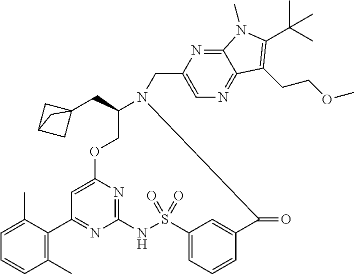 [SMILES: COCCc1c(C(C)(C)C)n(C)c2nc(CN3C(=O)c4cccc(c4)S(=O)(=O)Nc4nc(cc(-c5c(C)cccc5C)n4)OC[C@H]3CC34CC(C3)C4)cnc12] |
| IV-83 | 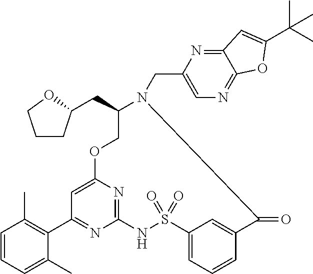 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@@H]1CCCO1)CO2] |
| IV-84 | 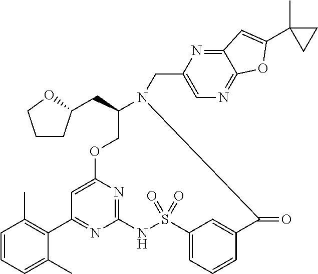 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@@H]1CCCO1)CO2] |
| IV-85 | 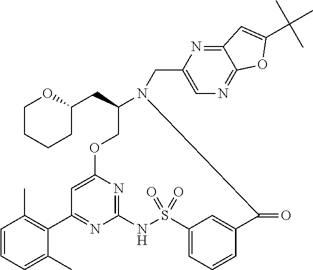 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@@H]1CCCCO1)CO2] |
| IV-86 | 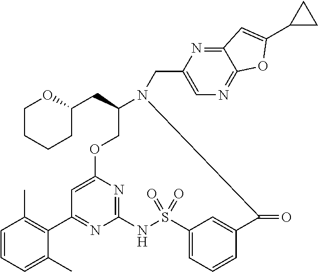 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCCO1)CO2] |
| IV-87 | 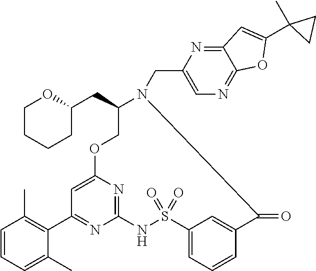 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@@H]1CCCCO1)CO2] |
| IV-88 | 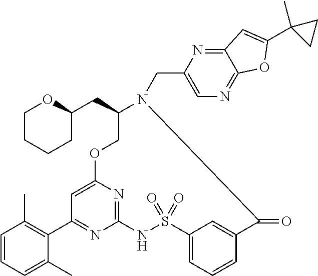 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@H]1CCCCO1)CO2] |
| IV-89 | 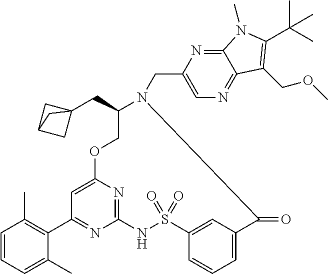 [SMILES: COCc1c(C(C)(C)C)n(C)c2nc(CN3C(=O)c4cccc(c4)S(=O)(=O)Nc4nc(cc(-c5c(C)cccc5C)n4)OC[C@H]3CC34CC(C3)C4)cnc12] |
| IV-90 | 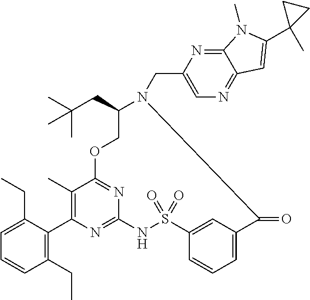 [SMILES: CCc1cccc(CC)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] |
| IV-91 | 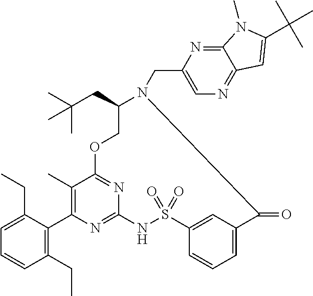 [SMILES: CCc1cccc(CC)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2] |
| IV-92 | 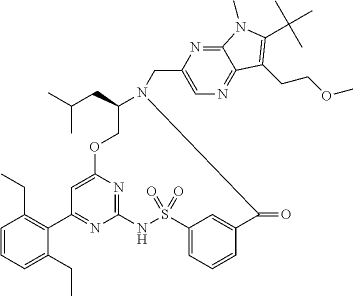 [SMILES: CCc1cccc(CC)c1-c1cc2nc(n1)NS(=O)(=O)c1cccc(c1)C(=O)N(Cc1cnc3c(CCOC)c(C(C)(C)C)n(C)c3n1)[C@H](CC(C)C)CO2] |
| IV-93 | 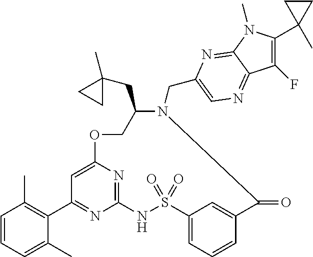 [SMILES: Cc1cccc(C)c1-c1cc2nc(n1)NS(=O)(=O)c1cccc(c1)C(=O)N(Cc1cnc3c(F)c(C4(C)CC4)n(C)c3n1)[C@H](CC1(C)CC1)CO2] |
| IV-94 | 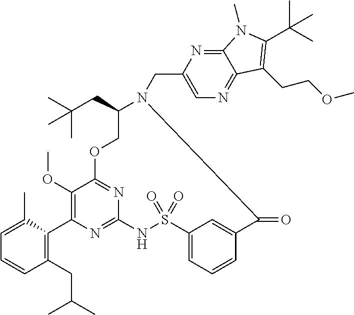 |
| IV-95 | 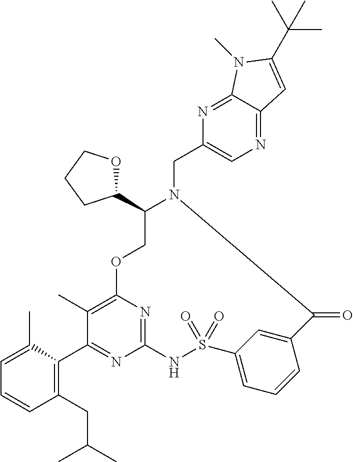 |
| IV-96 | 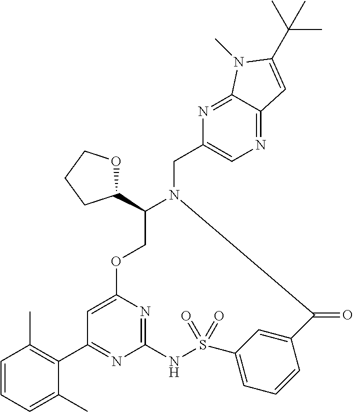 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H]([C@@H]1CCCO1)CO2] |
| IV-97 | 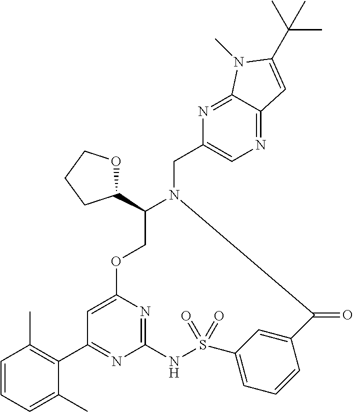 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H]([C@@H]1CCCO1)CO2] |
| IV-98 | 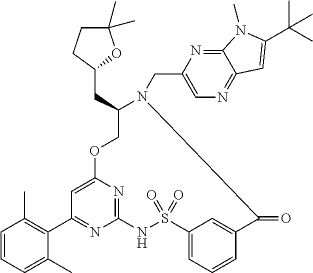 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](C[C@@H]1CCC(C)(C)O1)CO2] |
| IV-99 | 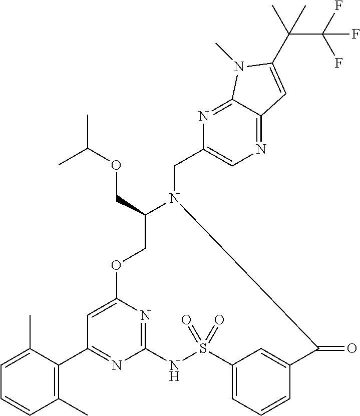 [SMILES: Cc1cccc(C)c1-c1cc2nc(n1)NS(=O)(=O)c1cccc(c1)C(=O)N(Cc1cnc3cc(C(C)(C)C(F)(F)F)n(C)c3n1)[C@H](COC(C)C)CO2] |
| IV-100 | 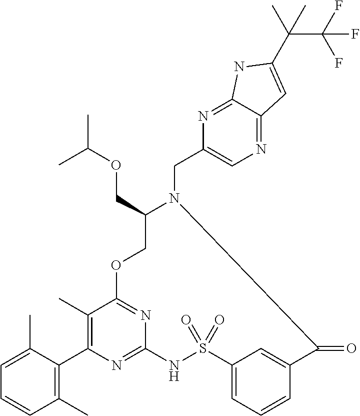 [SMILES: Cc1cccc(C)c1-c1nc2nc(c1C)OC[C@@H](COC(C)C)N(Cc1cnc3cc(C(C)(C)C(F)(F)F)[nH]c3n1)C(=O)c1cccc(c1)S(=O)(=O)N2] |
| IV-101 | 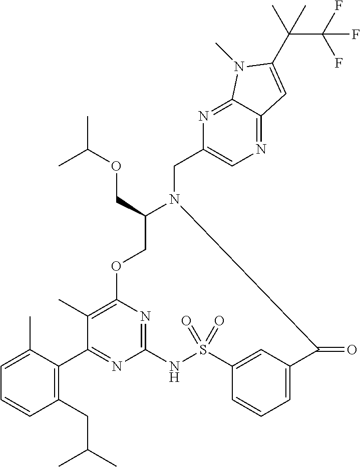 [SMILES: Cc1cccc(CC(C)C)c1-c1nc2nc(c1C)OC[C@@H](COC(C)C)N(Cc1cnc3cc(C(C)(C)C(F)(F)F)n(C)c3n1)C(=O)c1cccc(c1)S(=O)(=O)N2] |
| IV-102 | 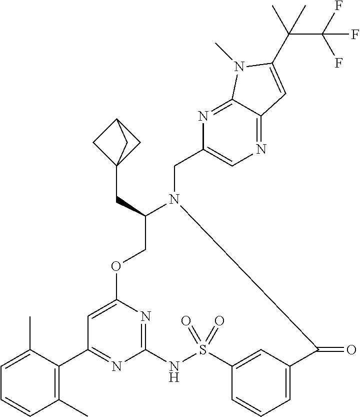 [SMILES: Cc1cccc(C)c1-c1cc2nc(n1)NS(=O)(=O)c1cccc(c1)C(=O)N(Cc1cnc3cc(C(C)(C)C(F)(F)F)n(C)c3n1)[C@H](CC13CC(C1)C3)CO2] |
| IV-103 | 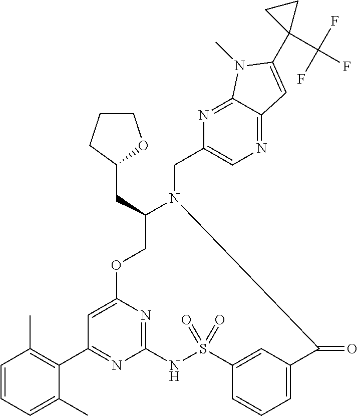 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](C[C@@H]1CCCO1)CO2] |
| IV-104 | 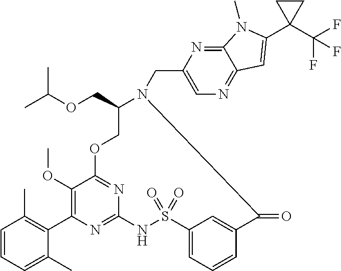 [SMILES: COc1c2nc(nc1-c1c(C)cccc1C)NS(=O)(=O)c1cccc(c1)C(=O)N(Cc1cnc3cc(C4(C(F)(F)F)CC4)n(C)c3n1)[C@H](COC(C)C)CO2] |
| IV-105 | 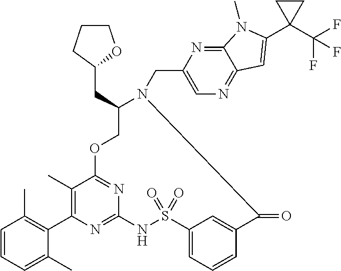 [SMILES: Cc1cccc(C)c1-c1nc2nc(c1C)OC[C@@H](C[C@@H]1CCCO1)N(Cc1cnc3cc(C4(C(F)(F)F)CC4)n(C)c3n1)C(=O)c1cccc(c1)S(=O)(=O)N2] |
| IV-106 | 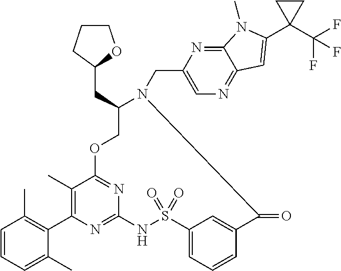 [SMILES: Cc1cccc(C)c1-c1nc2nc(c1C)OC[C@@H](C[C@H]1CCCO1)N(Cc1cnc3cc(C4(C(F)(F)F)CC4)n(C)c3n1)C(=O)c1cccc(c1)S(=O)(=O)N2] |

### Methods of Treatment

Any of the novel compounds disclosed herein, such as, for example, a compound chosen from compounds of any of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, Compounds IV-1 to IV-106, tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as, for example, a compound chosen from compounds of any of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, Compounds IV-1 to IV-106, tautomers thereof, deuterated derivatives of those compounds and tautomers, and pharmaceutically acceptable salts of any of the foregoing, alone or in combination with another active ingredient, such as one or more CFTR modulating agents. In some embodiments, the one or more CFTR modulating agents are selected from ivacaftor, deutivacaftor, lumacaftor, and tezacaftor. In some embodiments, the patient has an F508del/minimal function (MF) genotype, F508del/F508del genotype (homozygous for the F508del mutation), F508del/gating genotype, or F508del/residual function (RF) genotype. In some embodiments, the patient is heterozygous and has one F508del mutation. In some embodiments, the patient is homozygous for the N₁₃₀₃K mutation.

In some embodiments, 5 mg to 500 mg of a compound disclosed herein, a tautomer thereof, deuterated derivatives of the compound and tautomer, or a pharmaceutically acceptable salt of any of the foregoing are administered daily.

In some embodiments, the patient has at least one F508del mutation in the CFTR gene. In some embodiments, the patient has a CFTR gene mutation that is responsive to a compound, tautomer, deuterated derivative, or pharmaceutically acceptable salt of the invention based on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense | Q2X | L218X | Q525X | R792X | E1104X |
| mutations | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical splice | 185+1G→T | 711+5G→A | 1717-8G→A | 2622+1G→A | 3121-1G→A |
| mutations | 296+1G→A | 712-1G→T | 1717-1G→A | 2790-1G→C | 3500-2A→G |
| | 296+1G→T | 1248+1G→A | 1811+1G→C | 3040G→C | 3600+2insT |
| | | | | (G970R) | |
| | 405+1G→A | 1249-1G→A | 1811+1.6kbA→G | | 3850-1G→A |
| | 405+3A→C | 1341+1G→A | 1811+1643G→T | 3120G→A | 4005+1G→A |
| | 406-1G→A | 1525-2A→G | 1812-1G→A | 3120+1G→A | 4374+1G→T |
| | 621+1G→T | 1525-1G→A | 1898+1G→A | 3121-2A→G | |
| | 711+1G→T | | 1898+1G→C | | |
| Small (≤3 | 182delT | 1078delT | 1677delTA | 2711delT | 3737delA |
| nucleotide) | 306insA | 1119delA | 1782delA | 2732insA | 3791delC |
| insertion/deletion | 306delTAGA | 1138insG | 1824delA | 2869insG | 3821delT |
| (ins/del) | 365-366insT | 1154insTC | 1833delT | 2896insAG | 3876delA |
| frameshift | 394delTT | 1161delC | 2043delG | 2942insT | 3878delG |
| mutations | 442delA | 1213delT | 2143delT | 2957delT | 3905insT |
| | 444delA | 1259insA | 2183AA→G^{a} | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |
| Non-small (>3 | CFTRdele1 | CFTRdele16-17b | 1461ins4 | | |
| nucleotide) | CFTRdele2 | CFTRdele17a, 17b | 1924del7 | | |
| insertion/deletion | CFTRdele2,3 | CFTRdele17a-18 | 2055del9→A | | |
| (ins/del) | CFTRdele2-4 | CFTRdele19 | 2105- | | |
| frameshift | CFTRdele3-10,14b-16 | CFTRdele19-21 | 2117del13insAGAAA | | |
| mutations | CFTRdele4-7 | CFTRdele21 | 2372de18 | | |
| | CFTRdele4-11 | CFTRdele22-24 | 2721del11 | | |
| | CFTR50kbdel | CFTRdele22,23 | 2991del32 | | |
| | CFTRdup6b-10 | 124del23bp | 3667ins4 | | |
| | CFTRdele11 | 602del14 | 4010del4 | | |
| | CFTRdele13,14a | 852del22 | 4209TGTT→AA | | |
| | CFTRdele14b-17b | 991de15 | | | |
| Missense | A46D | V520F | Y569D | N1303K | |
| mutations that | G85E | A559T | L1065P | | |
| Are not | R347P | R560T | R1066C | | |
| responsive in | L467P | R560S | L1077P | | |
| vitro to TEZ, | I507del | A561E | M1101K | | |
| IVA, or | | | | | |
| TEZ/IVA | | | | | |
| and | | | | | |
| % PI >50% | | | | | |
| and SwCl- | | | | | |
| >86 mmol/L | | | | | |

^{a}Also known as 2183delAA→G.
CFTR: cystic fibrosis transmembrane conductance regulator;
IVA: ivacaftor.
SwCl: sweat chloride.
TEZ: tezacaftor.
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine, and chlorine, for example ²H, ³H, ¹³C, ¹⁴C, ¹⁵N, ¹⁸O, ¹⁷O, ³¹P, ³²P, ³⁵S, ¹⁸F, and ³⁶Cl, respectively.

The isotope-labelled compounds and salts can be used in a number of beneficial ways. They can be suitable for medicaments and/or various types of assays, such as substrate tissue distribution assays. For example, tritium (³H)— and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (2H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted as deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as, for example, compounds of any of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, Compounds IV-1 to IV-106, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein include Pulmozyme®.

In some embodiments, the additional agent is a bronchodilator. Exemplary bronchodilators include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreatic enzyme replacement), including Pancrease®, Pancreacarb®, Ultrase®, or Creon®, Liprotomase® (formerly Trizytek®), Aquadeks®, or glutathione inhalation. In some embodiments, the additional nutritional agent is pancrelipase.

In some embodiments, at least one additional active pharmaceutical ingredient is selected from CFTR modulating agents. In some embodiments, the at least one additional active pharmaceutical ingredient is selected from CFTR potentiators. In some embodiments, the potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol and deuterated derivatives and pharmaceutically acceptable salts of any of the foregoing. In some embodiments, the at least one additional active pharmaceutical ingredient is chosen from CFTR correctors. In some embodiments, the correctors are selected from lumacaftor, tezacaftor, and deuterated derivatives and pharmaceutically acceptable salts of any of the foregoing.

In some embodiments, the at least one additional active pharmaceutical ingredient is chosen from (a) tezacaftor, lumacaftor, and deuterated derivatives and pharmaceutically acceptable salts thereof; and/or (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts of any of the foregoing.

Thus, in some embodiments, the combination therapies provided herein comprise (a) a compound selected from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing; and (b) at least one compound selected from tezacaftor, lumacaftor, and deuterated derivatives and pharmaceutically acceptable salts thereof; or (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts of any of the foregoing. In other embodiments, the combination therapies provided herein comprise (a) at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, Compounds IV-1 to IV-106, tautomers thereof, deuterated derivatives of those compounds and tautomers, and pharmaceutically acceptable salts of any of the foregoing; (b) at least one compound selected from tezacaftor and pharmaceutically acceptable salts thereof; and (c) at least one compound selected from ivacaftor, deutivacaftor, and pharmaceutically acceptable salts thereof. In still other embodiments, the combination therapies provided herein comprise (a) at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to 1-265, Compounds II-1 to II-38, Compounds III-1 to 111-25, Compounds IV-1 to IV-106, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and/or (c) at least one compound selected from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof.

In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in combination with at least one compound chosen from tezacaftor and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to I-265, Compounds II-1 to II-38, Compound Ills-1 to III-25, Compounds IV-1 to IV-106, tautomers thereof, deuterated derivatives of those compounds and tautomers, and pharmaceutically acceptable salts of any of the foregoing, is administered in combination with at least one compound chosen from lumacaftor and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in combination with at least one compound chosen from ivacaftor and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to 1-265, Compounds II-1 to II-38, Compounds III-1 to 111-25, Compounds IV-1 to IV-106, tautomers thereof, deuterated derivatives of those compounds and tautomers, and pharmaceutically acceptable salts of any of the foregoing, is administered in combination with at least one compound chosen from deutivacaftor and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to 1-265, Compounds II-1 to II-38, Compounds III-1 to 111-25, Compounds IV-1 to IV-106, tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof.

In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to 1-265, Compounds II-1 to II-38, Compounds III-1 to 111-25, Compounds IV-1 to IV-106, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to 1-265, Compounds II-1 to II-38, Compounds III-1 to 111-25, Compounds IV-1 to IV-106, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof.

Each of the compounds of the disclosure, (e.g., compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing), independently can be administered once daily, twice daily, or three times daily. In some embodiments, at least one compound chosen from compounds of Formulae I, Ia, Ia(i), Ib, Ib(i), Ic, Ic(i), Id, Id(i), Ie, Ie(i), If, If(i), II, ITT, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered once daily. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered twice daily.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compound III-1 to III-25, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts of any of the foregoing are administered once daily. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof are administered twice daily.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof are administered once daily. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, at least one compound chosen from tezacaftor and pharmaceutically acceptable salts thereof, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof are administered twice daily.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof, and at least one compound chosen from lumacaftor and deuterated derivatives and pharmaceutically acceptable salts thereof, are administered once daily. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof, and at least one compound chosen from lumacaftor and deuterated derivatives and pharmaceutically acceptable salts thereof, are administered twice daily.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from ivacaftor and deuterated derivatives and pharmaceutically acceptable salts thereof, are administered twice daily. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least one compound chosen from lumacaftor and pharmaceutically acceptable salts thereof, are administered once daily and at least one compound chosen from ivacaftor and pharmaceutically acceptable salts thereof, are administered twice daily.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof, are administered once or twice daily. In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof, are administered once daily and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof, are administered once or twice daily.

Compounds of the disclosure, (e.g., compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing), as well as additional CFTR modulator compounds, such as, e.g., tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]-nonadeca-1(18),2,4,14,16-pentaen-6-ol, and deuterated derivatives and pharmaceutically acceptable salts thereof, can be administered in a single pharmaceutical composition or separate pharmaceutical compositions. Such pharmaceutical compositions can be administered once daily or multiple times daily, such as twice daily. As used herein, the phrase that a given amount of API (e.g., tezacaftor, lumacaftor, ivacaftor, deutivacaftor (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof) is administered once or twice daily or per day means that said given amount is administered per dosing once or twice daily.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; and at least one compound chosen from ivacaftor and deuterated derivatives and pharmaceutically acceptable salts thereof is administered in a third pharmaceutical composition.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from deutivacaftor and deuterated derivatives and pharmaceutically acceptable salts thereof is administered in a third pharmaceutical composition.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof is administered in a third pharmaceutical composition.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in a first pharmaceutical composition; at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof is administered in a second pharmaceutical composition; at least one compound chosen from lumacaftor and deuterated derivatives and pharmaceutically acceptable salts thereof is administered in a third pharmaceutical composition.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, is administered in a first pharmaceutical composition; and at least one compound chosen from tezacaftor and deuterated derivatives and pharmaceutically acceptable salts thereof and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof are administered in a second pharmaceutical composition. In some embodiments, the second pharmaceutical composition comprises a half of a daily dose of said at least one compound chosen from ivacaftor and deuterated derivatives and pharmaceutically acceptable salts thereof, and the other half of the daily dose of said at least one compound chosen from ivacaftor and deuterated derivatives and pharmaceutically acceptable salts thereof is administered in a third pharmaceutical composition.

In some embodiments, at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, at least one compound chosen from tezacaftor and pharmaceutically acceptable salts thereof, and at least one compound chosen from ivacaftor, deutivacaftor, and pharmaceutically acceptable salts thereof are administered in a first pharmaceutical composition. In some embodiments, the first pharmaceutical composition is administered to the patient twice daily. In some embodiments, the first pharmaceutical composition is administered once daily. In some embodiments, the first pharmaceutical composition is administered once daily and, when the first composition comprises ivacaftor, a second composition comprising only ivacaftor is administered once daily.

Any suitable pharmaceutical compositions can be used for compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, tezacaftor, ivacaftor, deutivacaftor, lumacaftor and tautomers thereof, deuterated derivatives of those compounds and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, all of which is incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for deutivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, all of which are incorporated herein by reference.

### Pharmaceutical Compositions

Another aspect of the disclosure provides a pharmaceutical composition comprising at least one novel compound of the disclosure (e.g., a compound chosen from compounds of Formulae I, Ia, Ia(i), Ib, Ib(i), Ic, Ic(i), Id, Id(i), Ie, Ie(i), If, If(i), II, III, and IV, Compounds I-1 to 1-265, Compounds II-1 to II-38, Compounds III-1 to 111-25, Compounds IV-1 to IV-106, tautomers thereof, deuterated derivatives of those compounds and tautomers, and pharmaceutically acceptable salts of any of the foregoing), and at least one pharmaceutically acceptable carrier.

In some embodiments, the disclosure provides pharmaceutical compositions comprising at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, (b) at least one compound chosen from tezacaftor and pharmaceutically acceptable salts thereof, and (c) at least one pharmaceutically acceptable carrier.

In some embodiments, the disclosure provides a pharmaceutical composition comprising (a) at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]-nonadeca-1(18),2,4,14,16-pentaen-6-ol, and deuterated derivatives and pharmaceutically acceptable salts of any of the foregoing, and (c) at least one pharmaceutically acceptable carrier.

In some embodiments, the disclosure provides a pharmaceutical composition comprising (a) at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from ivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from deutivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol, and deuterated derivatives and pharmaceutically acceptable salts of any of the foregoing, (c) at least one compound chosen from lumacaftor and deuterated derivatives and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose, and cellulose acetate), powdered tragacanth, malt, gelatin, talc, excipients (such as cocoa butter and suppository waxes), oils (such as peanut oil, cottonseed oil, safflower oil, sesame oil, olive oil, corn oil, and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### LIST OF EXEMPLARY EMBODIMENTS

1. A compound of Formula I:

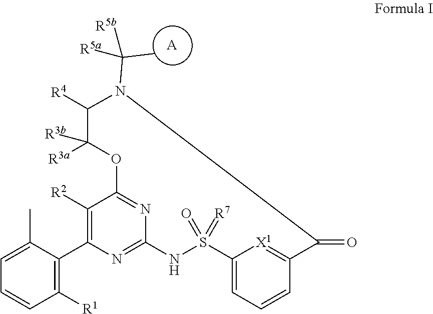

tautomers thereof, deuterated derivatives of those compounds and tautomers, and pharmaceutically acceptable salts of any of the foregoing, wherein:
Ring A is

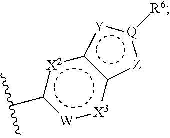

Q is selected from —C— and —N—;
W is selected from —CH—, —C(F)—, —C(CF₃)—, and —N—;
X¹, X², and X³ are each independently selected from —CH— and —N—;
Y is selected from —N—, —N(R^{y})—, —C(R^{y})—, and —O—, wherein
   R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CH—, —O—, —S—, —S(O)—, —S(O)₂—, —N—, and —N^{z}, wherein
   R^{z} is selected from hydrogen and C₁-C₈ alkyl;
R¹ is selected from: C₃-C₆ cycloalkyl, C₁-C₈ alkoxy, and C₁-C₈ alkyl (which may be optionally substituted with a group selected from C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   OC₃-C₇ cycloalkyl (which may be optionally substituted with 1 to 2 groups selected from C₁-C₈ alkyl, C₁-C₈ alkoxy, halogen, and C₁-C₈ haloalkyl);
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁶ is selected from halogen, 4- to 6-membered heterocyclyl, C₃-C₈ cycloalkyl (which may be optionally substituted with a group selected from C₁-C₈ alkyl, C₁-C₈ haloalkyl, and halogen); and C₁-C₈ alkyl (which may be optionally substituted with 1 to 2 groups independently selected from C₁-C₈ alkoxy, halogen, oxo, —OH, —NH₂, and —SO₂CH₃); and
R⁷ is selected from O, and NR, wherein
   R is selected from hydrogen and C₁-C₈ alkyl;
      with the proviso that wherein the compound of Formula I is not selected from:

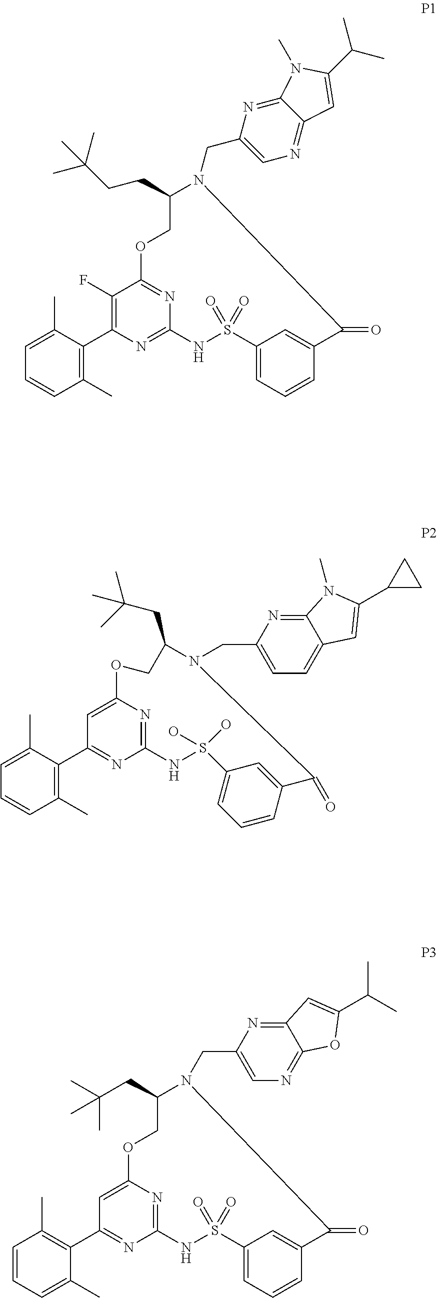 [SMILES: Cc1cccc(C)c1-c1nc2nc(c1F)OC[C@@H](CCC(C)(C)C)N(Cc1cnc3cc(C(C)C)n(C)c3n1)C(=O)c1cccc(c1)S(=O)(=O)N2 ; Cc1cccc(C)c1-c1cc2nc(n1)NS(O)(O)c1cccc(c1)C(=O)N(Cc1ccc3cc(C4CC4)n(C)c3n1)[C@H](CC(C)(C)C)CO2 ; Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2]

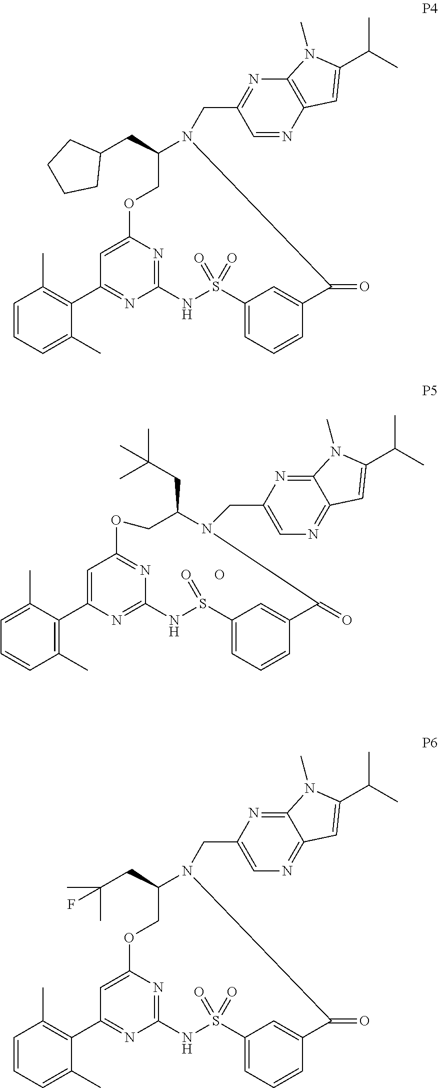

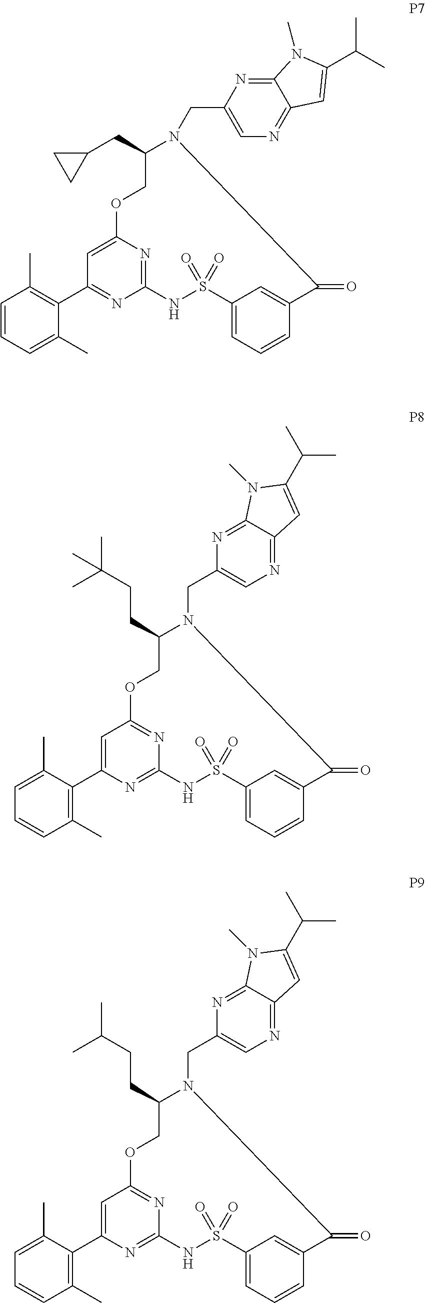 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CCC(C)C)CO2]

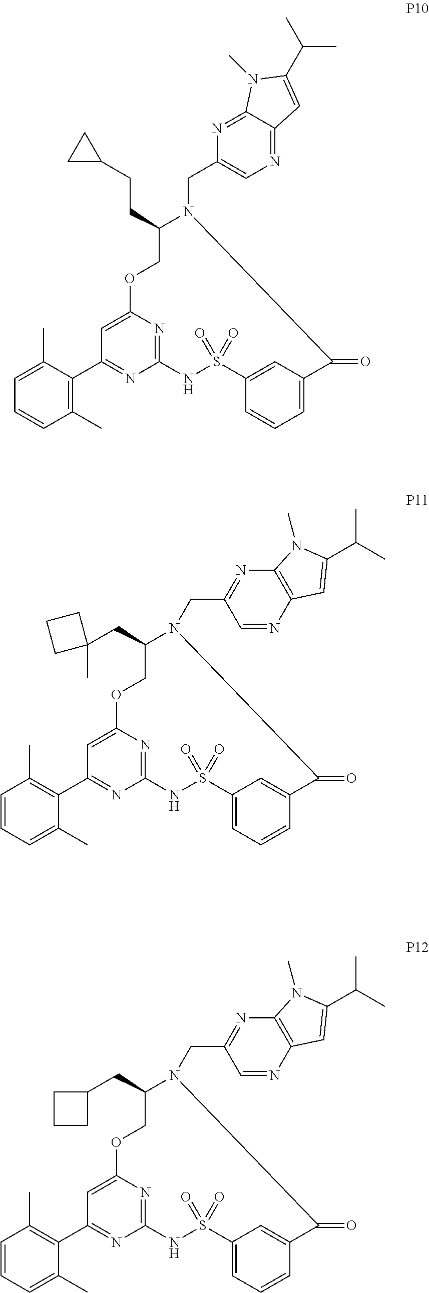 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1(C)CCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1CCC1)CO2]

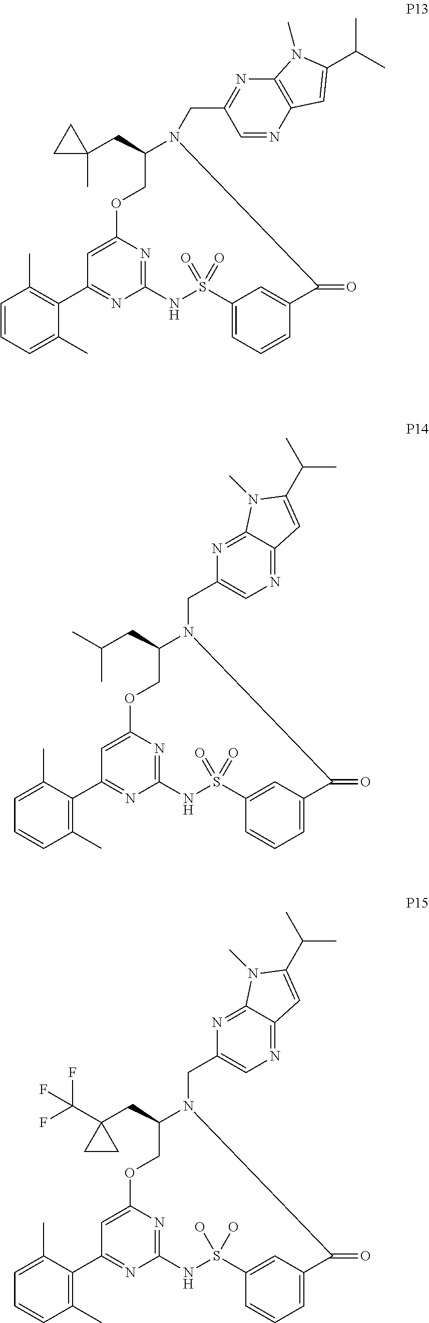 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)C)CO2 ; Cc1cccc(C)c1-c1cc2nc(n1)NS(O)(O)c1cccc(c1)C(=O)N(Cc1cnc3cc(C(C)C)n(C)c3n1)[C@H](CC1(C(F)(F)F)CC1)CO2]

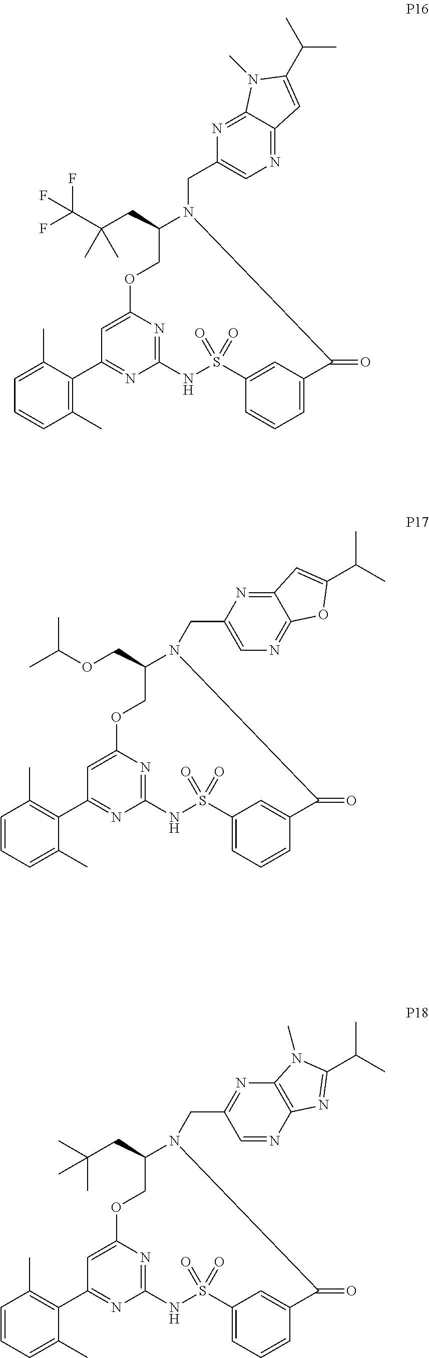 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C(F)(F)F)CO2 ; Cc1cccc(C)c1-c1cc2nc(n1)NS(=O)(=O)c1cccc(c1)C(=O)N(Cc1cnc3oc(C(C)C)cc3n1)[C@H](COC(C)C)CO2 ; Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CO2]

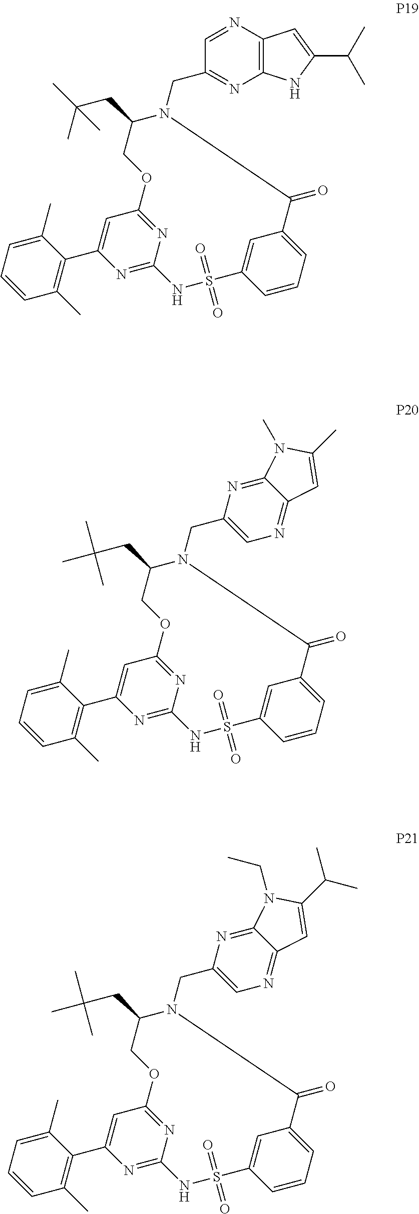 [SMILES: Cc1cccc(C)c1-c1cc2nc(n1)NS(=O)(=O)c1cccc(c1)C(=O)N(Cc1cnc3cc(C(C)C)[nH]c3n1)[C@H](CC(C)(C)C)CO2 ; Cc1cccc(C)c1-c1cc2nc(n1)NS(=O)(=O)c1cccc(c1)C(=O)N(Cc1cnc3cc(C)n(C)c3n1)[C@H](CC(C)(C)C)CO2 ; CCn1c(C(C)C)cc2ncc(CN3C(=O)c4cccc(c4)S(=O)(=O)Nc4nc(cc(-c5c(C)cccc5C)n4)OC[C@H]3CC(C)(C)C)nc21]

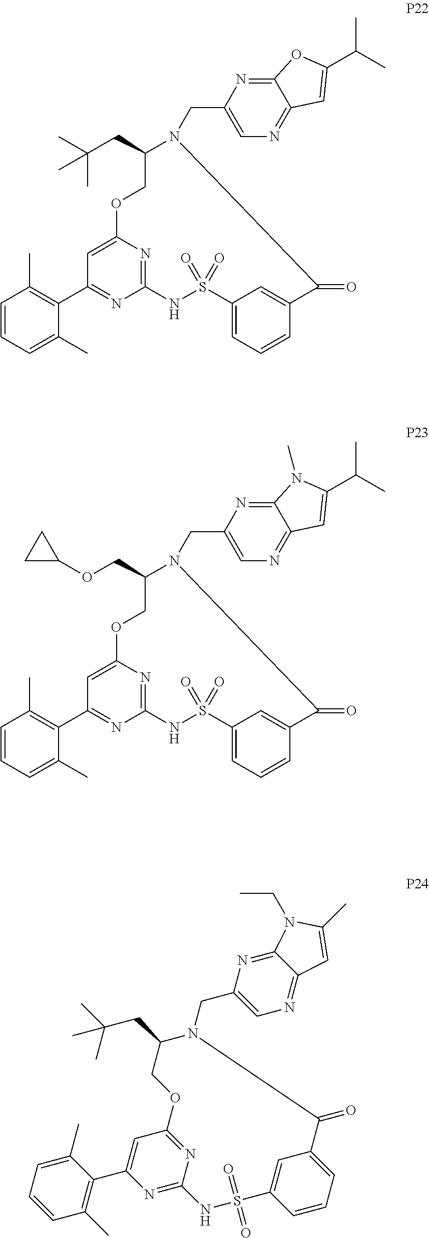 [SMILES: Cc1cccc(C)c1-c1cc2nc(n1)NS(=O)(=O)c1cccc(c1)C(=O)N(Cc1cnc3cc(C(C)C)oc3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](COC1CC1)CO2 ; CCn1c(C)cc2ncc(CN3C(=O)c4cccc(c4)S(=O)(=O)Nc4nc(cc(-c5c(C)cccc5C)n4)OC[C@H]3CC(C)(C)C)nc21]

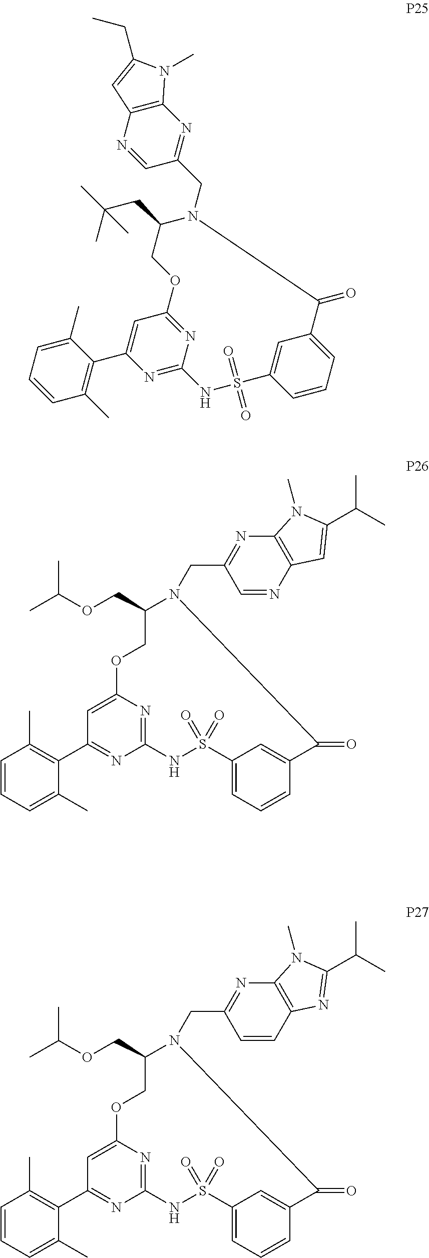 [SMILES: CCc1cc2ncc(CN3C(=O)c4cccc(c4)S(=O)(=O)Nc4nc(cc(-c5c(C)cccc5C)n4)OC[C@H]3CC(C)(C)C)nc2n1C ; Cc1cccc(C)c1-c1cc2nc(n1)NS(=O)(=O)c1cccc(c1)C(=O)N(Cc1cnc3cc(C(C)C)n(C)c3n1)[C@H](COC(C)C)CO2 ; Cc1cccc(C)c1-c1cc2nc(n1)NS(=O)(=O)c1cccc(c1)C(=O)N(Cc1ccc3nc(C(C)C)n(C)c3n1)[C@H](COC(C)C)CO2]

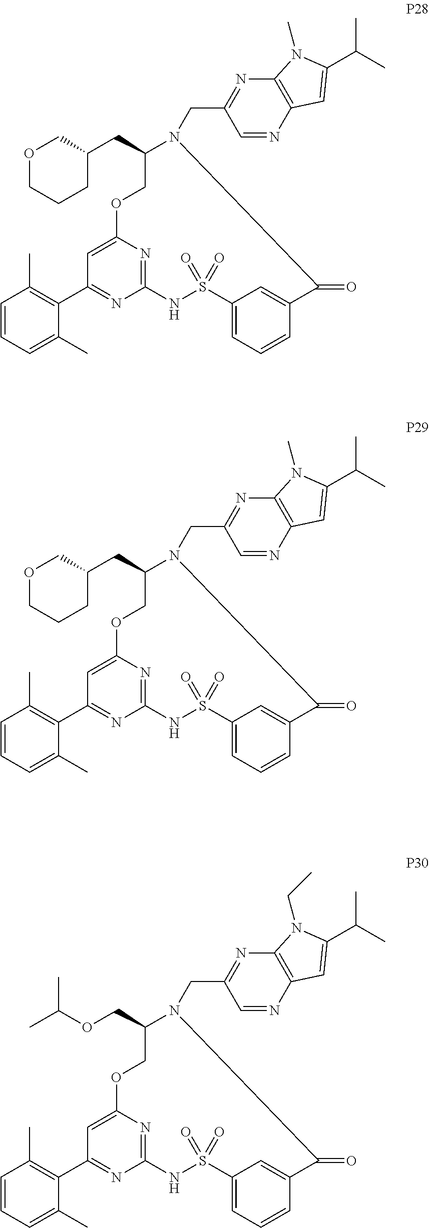 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; CCn1c(C(C)C)cc2ncc(CN3C(=O)c4cccc(c4)S(=O)(=O)Nc4nc(cc(-c5c(C)cccc5C)n4)OC[C@H]3COC(C)C)nc21]

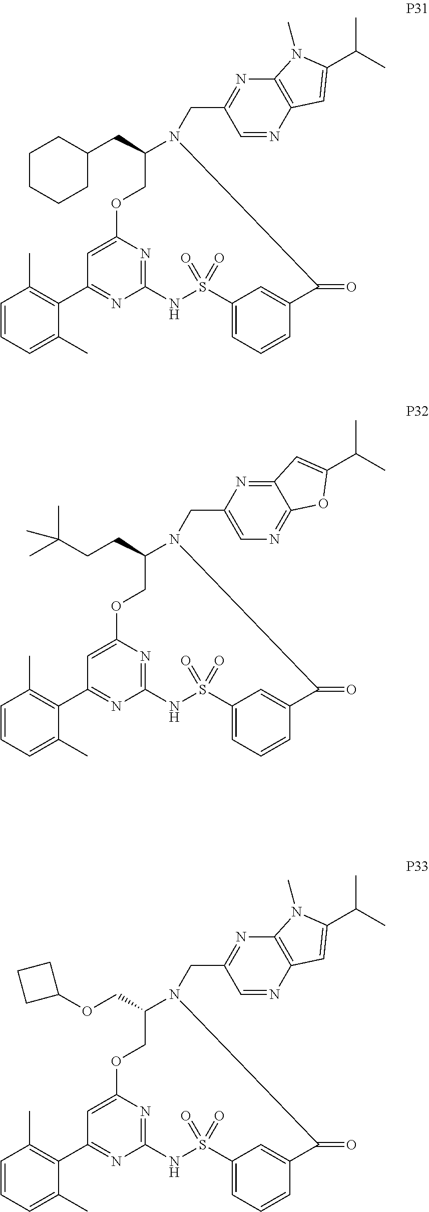 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CCC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2]

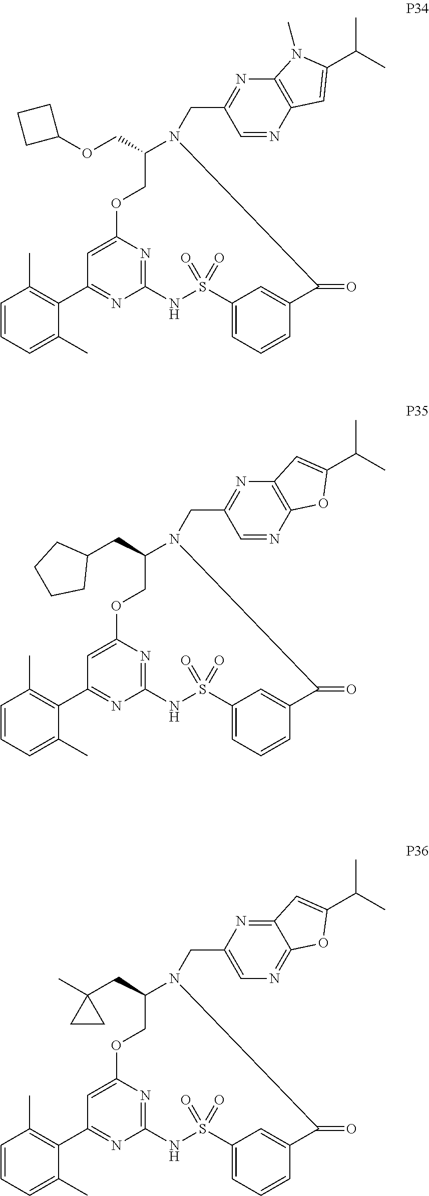 [SMILES: Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1CCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2]

and tautomers, deuterated derivatives, and pharmaceutically acceptable salts thereof.
2. The compound, tautomer, deuterated derivative, or salt of Embodiment 1, wherein R¹ is selected from C₁-C₄ alkyl.
3. The compound, tautomer, deuterated derivative, or salt of Embodiment 1, wherein R¹ is CH₃.
4. The compound, tautomer, deuterated derivative, or salt of any one of Embodiments 1 to 3, wherein R² is selected from: hydrogen, C₁-C₄ alkyl, C₁-C₄ haloalkyl, and C₁-C₄ alkoxy.
5. The compound, tautomer, deuterated derivative, or salt of any one of Embodiments 1 to 4, wherein R² is selected from: hydrogen and C₁-C₄ alkyl.
6. The compound, tautomer, deuterated derivative, or salt of any one of Embodiments 1 to 5, wherein R² is selected from: hydrogen and methyl.
7. The compound, tautomer, deuterated derivative, or salt of Embodiment 1, wherein R^{3a} and R^{3b} are independently selected from hydrogen, halogen, C₁-C₄ alkyl, and C₃-C₇ cycloalkyl.
8. The compound, tautomer, deuterated derivative, or salt of Embodiment 1, wherein R^{3a} and R^{3b} are hydrogen.
9. The compound, tautomer, deuterated derivative, or salt of any one of Embodiments 1 to 8, wherein R⁴ is selected from C₁-C₆ alkyl optionally substituted with a group selected from halogen, haloalkyl and C₁-C₄ alkoxy.
10. The compound, tautomer, deuterated derivative, or salt of any one of Embodiments 1 to 8, wherein R⁴ is selected from C₁-C₄ alkyl substituted with from C₃-C₅ cycloalkyl.
11. The compound, tautomer, deuterated derivative, or salt of any one of Embodiments 1 to 8, wherein R⁴ is selected from C₁-C₄ alkyl substituted with 1 to 2 groups independently selected from C₃-C₅ cycloalkyl (which may be optionally substituted with 1 to 2 groups selected from halogen, haloalkyl, and C₁-C₄ alkyl).
12. The compound, tautomer, deuterated derivative, or salt of any one of Embodiments 1 to 8, wherein R⁴ is selected from C₁-C₆ alkyl optionally substituted with phenyl (which may be optionally substituted with a group selected from C₁-C₄ alkyl, C₁-C₄ alkoxy, halogen, and haloalkyl).
13. The compound, tautomer, deuterated derivative, or salt of any one of Embodiments 1 to 8, wherein R⁴ is selected from C₁-C₆ alkyl substituted with a 4- to 6-membered heterocyclyl (which may be optionally substituted with 1 to 2 groups independently selected from halogen, haloalkyl, and C₁-C₄ alkyl).
14. The compound, tautomer, deuterated derivative, or salt of any one of Embodiments 1 to 8, wherein R⁴ is selected from:

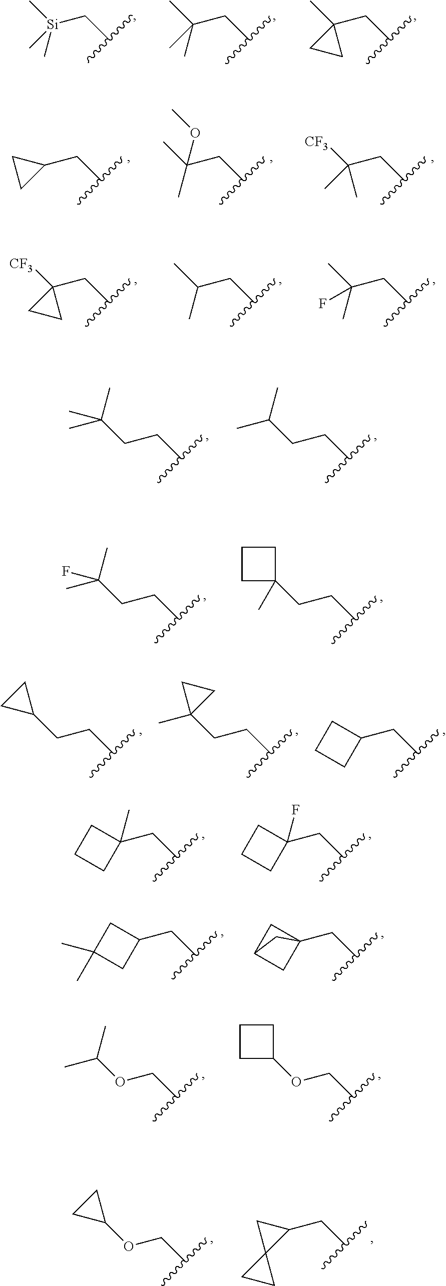

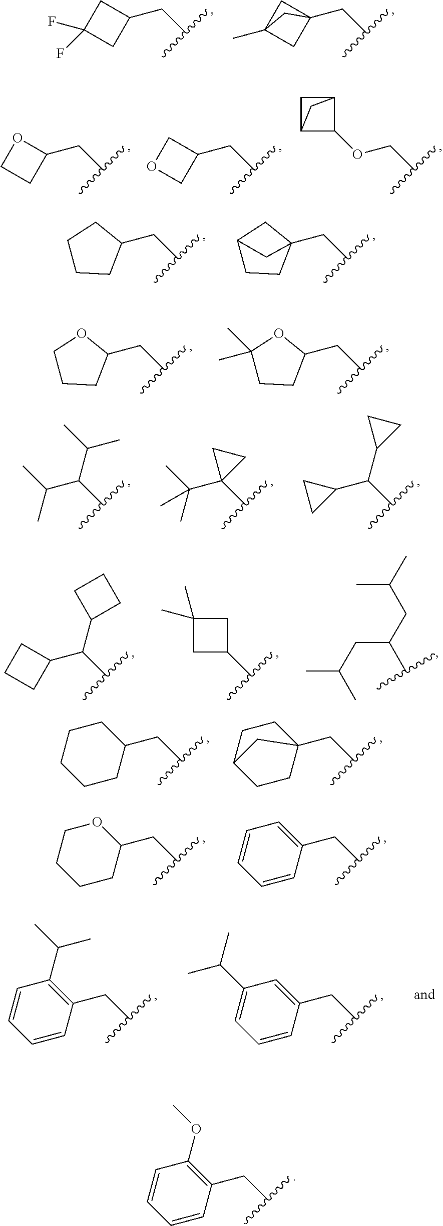

15. The compound, tautomer, deuterated derivative, or salt of any one of Embodiments 1-14, wherein R^{5a} and R^{5b} are independently selected from hydrogen, halogen, C₁-C₄ alkyl, and C₃-C₇ cycloalkyl.
16. The compound, tautomer, deuterated derivative, or salt of any one of Embodiments 1-15, wherein R^{5a} and R^{5b} are hydrogen.
17. The compound, tautomer, deuterated derivative, or salt of any one of Embodiments 1 to 16, wherein R⁶ is selected from C₃-C₈ cycloalkyl (which may be optionally substituted with a group selected from C₁-C₄ alkyl, haloalkyl, and halogen)
18. The compound, tautomer, deuterated derivative, or salt of any one of Embodiments 1 to 16, wherein R⁶ is selected from C₃-C₆ cycloalkyl (which may be optionally substituted with a group selected from C₁-C₄ alkyl).
19. The compound, tautomer, deuterated derivative, or salt of any one of Embodiments 1 to 16, wherein R⁶ is selected from C₁-C₆ alkyl (which may be optionally substituted a group selected from C₁-C₄ alkoxy, halogen, —OH, oxo, —NH₂, and —SO₂CH₃).
20. The compound, tautomer, deuterated derivative, or salt of any one of Embodiments 1 to 16, wherein R⁶ is selected from C₁-C₆ alkyl and C₃-C₆ cycloalkyl.
21. The compound, tautomer, deuterated derivative, or salt of any one of Embodiments 1 to 16, wherein R⁶ is selected from C₁-C₄ alkyl (which may be optionally substituted with —OH) and C₄ cycloalkyl (which may be optionally substituted with a group selected from C₁-C₄ alkyl).
22. The compound, tautomer, deuterated derivative, or salt of any one of Embodiments 1 to 16, wherein R⁶ is selected from C₃ alkyl and C₃ cycloalkyl (which may be optionally substituted with a group selected from C₁-C₄ alkyl).
23. The compound, tautomer, deuterated derivative, or salt of any one of Embodiments 1 to 16, wherein R⁶ is selected from C₃-C₄ cycloalkyl, optionally substituted with methyl.
24. The compound, tautomer, deuterated derivative, or salt of any one of Embodiments 1 to 16, wherein R⁶ is selected from:

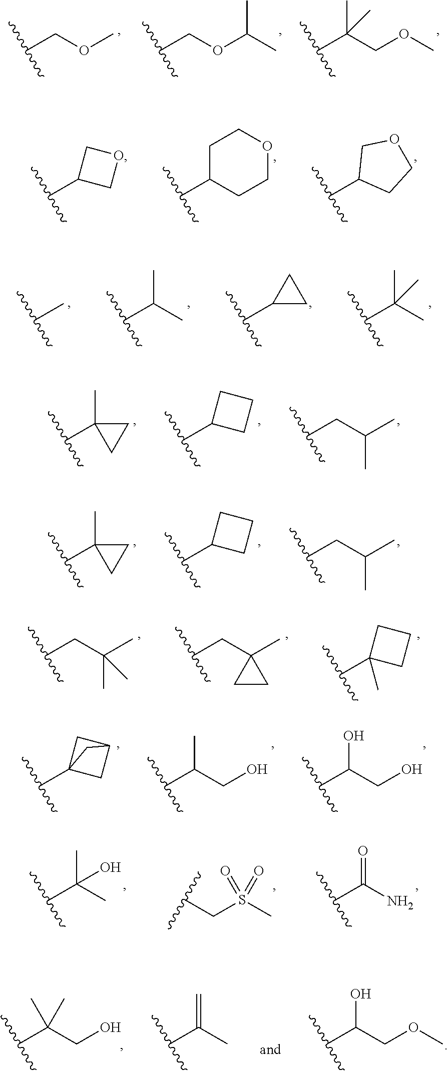

25. The compound, tautomer, deuterated derivative, or salt of any one of Embodiments 1 to 24, wherein R⁷ is O.
26. The compound, tautomer, deuterated derivative, or salt of any one of Embodiments 1 to 24, wherein X¹ is C.
26. The compound, tautomer, deuterated derivative, or salt of any one of Embodiments 1 to 24, wherein Q is C.
27. The compound, tautomer, deuterated derivative, or salt of Embodiment 1, selected from compounds of Formula Ia:

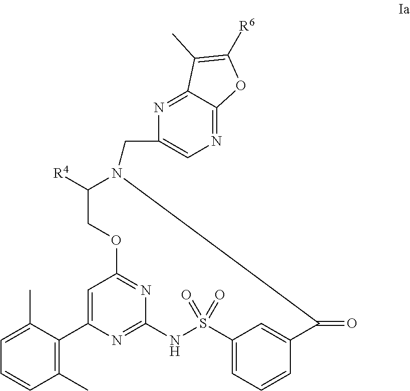

and tautomers thereof, or deuterated derivatives of the compounds and tautomers, and pharmaceutically acceptable salts of any of the foregoing, wherein R⁴ and R⁶ are as defined in Embodiment 1.
28. The compound, tautomer, deuterated derivative, or salt of Embodiment 1, selected from compounds of Formula Ia(i):

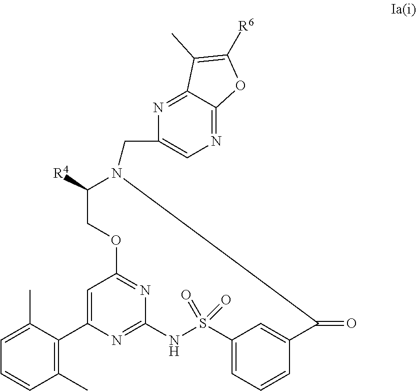

and tautomers thereof, or deuterated derivatives of the compounds and tautomers, and pharmaceutically acceptable salts of any of the foregoing, wherein R⁴ and R⁶ are as defined in Embodiment 1.
29. The compound, tautomer, deuterated derivative, or salt of Embodiment 1, selected from compounds of Formula Ib:

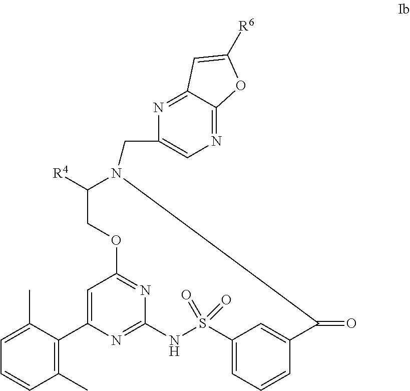

and tautomers thereof, or deuterated derivatives of the compounds and tautomers, and pharmaceutically acceptable salts of any of the foregoing, wherein R⁴ and R⁶ are as defined in Embodiment 1.
30. The compound, tautomer, deuterated derivative, or salt of Embodiment 1, selected from compounds of Formula Ib(i):

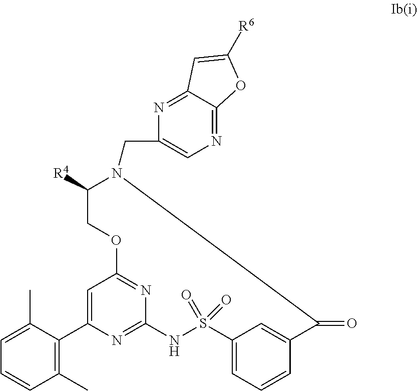

and tautomers thereof, or deuterated derivatives of the compounds and tautomers, and pharmaceutically acceptable salts of any of the foregoing, wherein R⁴ and R⁶ are as defined in Embodiment 1.
31. The compound, tautomer, deuterated derivative, or salt of Embodiment 1, selected from compounds of Formula Ic:

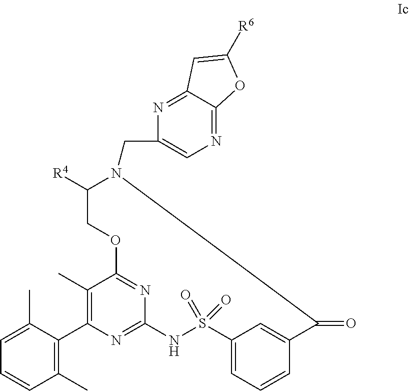

and tautomers thereof, or deuterated derivatives of the compounds and tautomers, and pharmaceutically acceptable salts of any of the foregoing, wherein R⁴ and R⁶ are as defined in Embodiment 1.
32. The compound, tautomer, deuterated derivative, or salt of Embodiment 1, selected from compounds of Formula Ic(i):

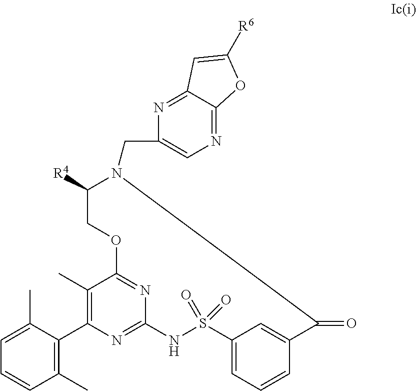

and tautomers thereof, or deuterated derivatives of the compounds and tautomers, and pharmaceutically acceptable salts of any of the foregoing, wherein R⁴ and R⁶ are as defined in Embodiment 1.
33. The compound, tautomer, deuterated derivative, or salt of Embodiment 1, selected from compounds of Formula Id:

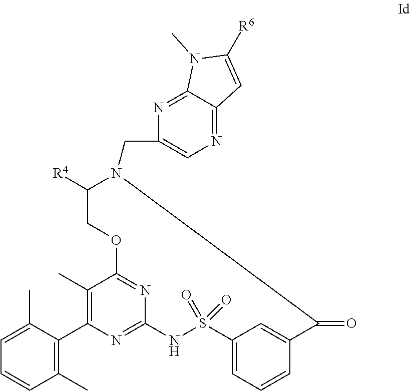

and tautomers thereof, or deuterated derivatives of the compounds and tautomers, and pharmaceutically acceptable salts of any of the foregoing, wherein R⁴ and R⁶ are as defined in Embodiment 1.
34. The compound, tautomer, deuterated derivative, or salt of Embodiment 1, selected from compounds of Formula Id(i):

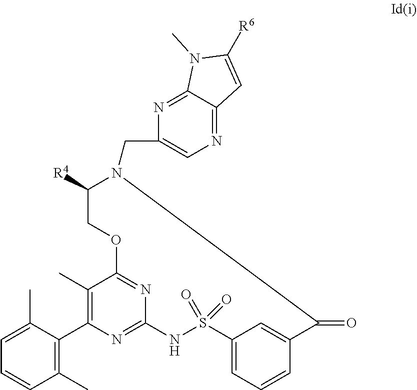

and tautomers thereof, or deuterated derivatives of the compounds and tautomers, and pharmaceutically acceptable salts of any of the foregoing, wherein R⁴ and R⁶ are as defined in Embodiment 1.
35. The compound, tautomer, deuterated derivative, or salt of Embodiment 1, selected from compounds of Formula Ie:

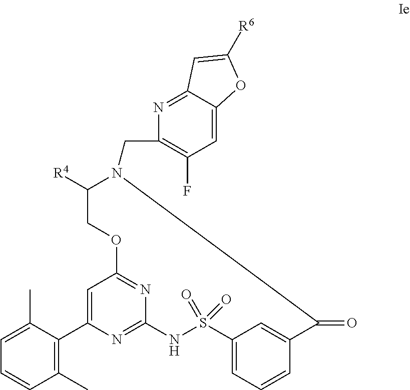

and tautomers thereof, or deuterated derivatives of the compounds and tautomers, and pharmaceutically acceptable salts of any of the foregoing, wherein R⁴ and R⁶ are as defined in Embodiment 1.
36. The compound, tautomer, deuterated derivative, or salt of Embodiment 1, selected from compounds of Formula Ie(i):

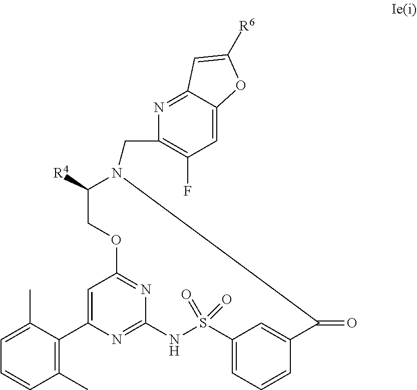

and tautomers thereof, or deuterated derivatives of the compounds and tautomers, and pharmaceutically acceptable salts of any of the foregoing, wherein R⁴ and R⁶ are as defined in Embodiment 1.
37. The compound, tautomer, deuterated derivative, or salt of Embodiment 1, selected from compounds of Formula If:

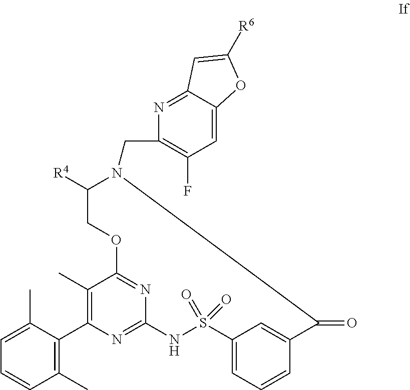

and tautomers thereof, or deuterated derivatives of the compounds and tautomers, and pharmaceutically acceptable salts of any of the foregoing, wherein R⁴ and R⁶ are as defined in Embodiment 1.
38. The compound, tautomer, deuterated derivative, or salt of Embodiment 1, selected from compounds of Formula If(i):

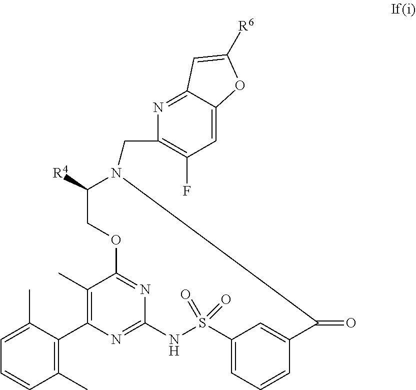

and tautomers thereof, or deuterated derivatives of the compounds and tautomers, and pharmaceutically acceptable salts of any of the foregoing, wherein R⁴ and R⁶ are as defined in Embodiment 1.
39. The compound, tautomer, deuterated derivative, or salt of Embodiment 1, wherein Ring A is selected from:

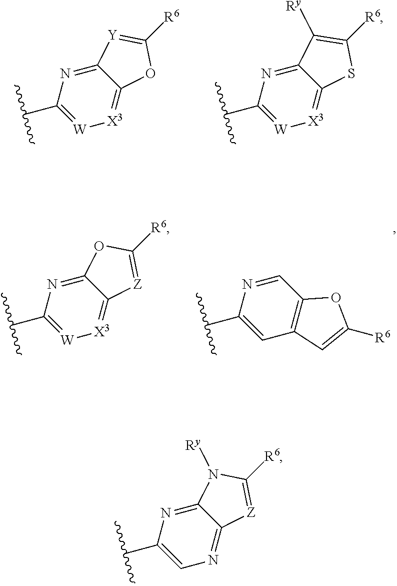

and wherein R⁶, W, X², X³, Y, R^{y}, and Z, are as defined in Embodiment 1.
40. The compound, tautomer, deuterated derivative, or salt of Embodiment 1, wherein Ring A is selected from:

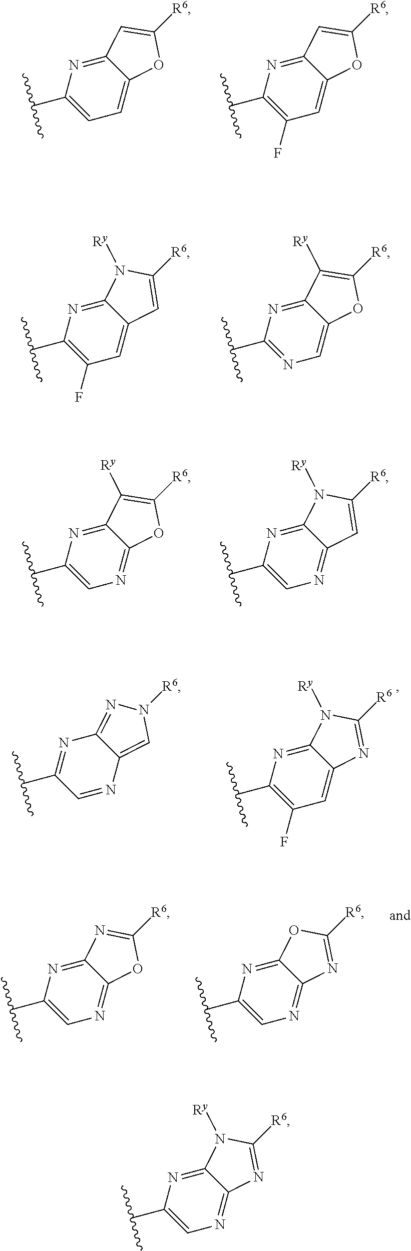

wherein R^{y} and R⁶ are as defined in Embodiment 1.
41. The compound, tautomer, deuterated derivative, or salt of Embodiment 1, 39, or 40, wherein R^{y} is selected from:

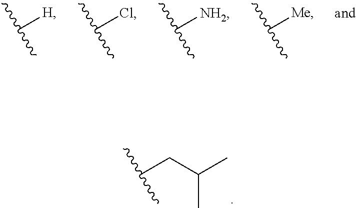

42. The compound, tautomer, deuterated derivative, or salt of Embodiment 1, wherein Ring A is chosen from:

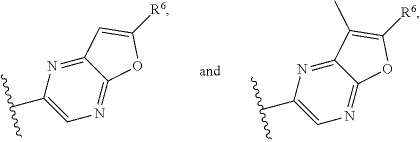

wherein R⁶ are as defined in Embodiment 1, and R⁴ is selected from C₁-C₆ alkyl substituted with from C₃-C₆ cycloalkyl (which is optionally substituted with methyl).
43. The compound, tautomer, deuterated derivative, or salt of any one of Embodiments 27-41, wherein R⁴ is selected from C₁-C₆ alkyl, optionally substituted with 1 to 3 halogens.
44. The compound, tautomer, deuterated derivative, or salt of any one of Embodiments 27-41, wherein R⁴ is selected from C₁-C₆ alkyl substituted with C₁-C₄ alkoxy.
45. The compound, tautomer, deuterated derivative, or salt of any one of Embodiments 27-41, wherein R⁴ is selected from C₁-C₆ alkyl substituted with C₃-C₆ cycloalkyl (which is optionally substituted with methyl).
46. The compound, tautomer, deuterated derivative, or salt of any one of Embodiments 27-41, wherein R⁴ is selected from C₁-C₆ alkyl substituted with a 5- to 6-membered heterocyclyl.
47. The compound, tautomer, deuterated derivative, or salt of any one of Embodiments 27-41, wherein R⁴ is selected from C₁-C₆ alkyl substituted with OC₃-C₆ cycloalkyl.
48. A compound selected from Compounds I-1 to I-265, tautomers thereof, deuterated derivatives of the compounds and tautomers, and pharmaceutically acceptable salts of any of the foregoing.
49. The compound according to Embodiment 48, selected from Compound I-4:

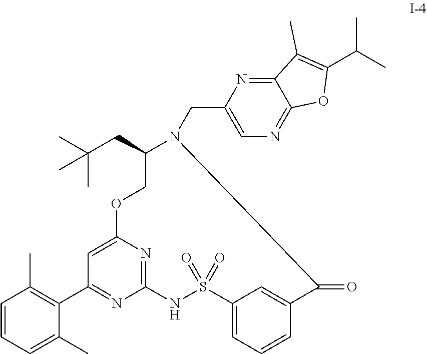 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)C)CO2]

and tautomers thereof, deuterated derivatives of the compounds and tautomers, and pharmaceutically acceptable salts of any of the foregoing.
50. The compound according to Embodiment 48, selected from Compound I-23:

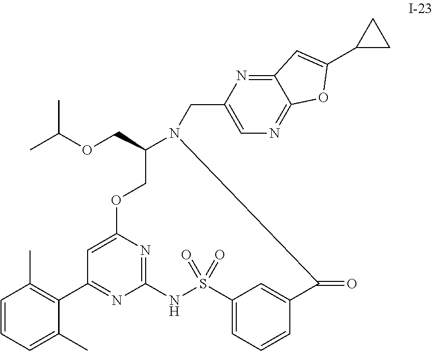 [SMILES: Cc1cccc(C)c1-c1cc2nc(n1)NS(=O)(=O)c1cccc(c1)C(=O)N(Cc1cnc3oc(C4CC4)cc3n1)[C@H](COC(C)C)CO2]

and tautomers thereof, deuterated derivatives of the compounds and tautomers, and pharmaceutically acceptable salts of any of the foregoing.
51. The compound according to Embodiment 48, selected from Compound I-34:

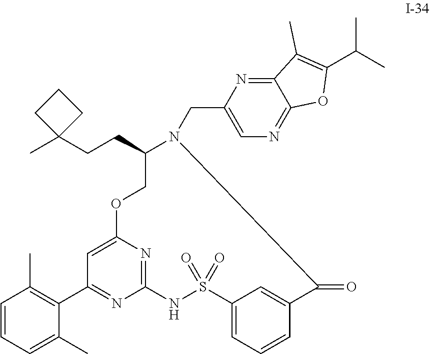 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC1(C)CCC1)CO2]

and tautomers thereof, deuterated derivatives of the compounds and tautomers, and pharmaceutically acceptable salts of any of the foregoing.
52. The compound according to Embodiment 48, selected from Compound I-35:

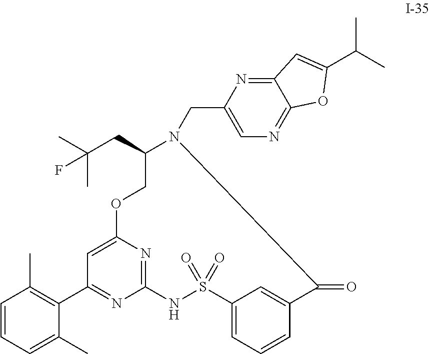 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)F)CO2]

and tautomers thereof, deuterated derivatives of the compounds and tautomers, and pharmaceutically acceptable salts of any of the foregoing.
53. The compound according to Embodiment 48, selected from Compound I-40:

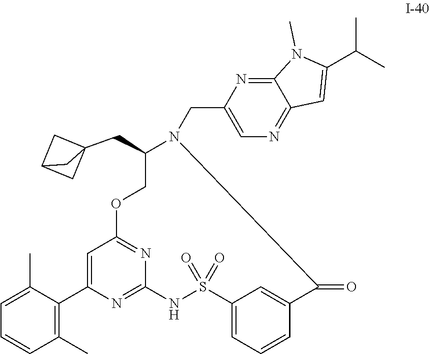 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC13CC(C1)C3)CO2]

and tautomers thereof, deuterated derivatives of the compounds and tautomers, and pharmaceutically acceptable salts of any of the foregoing.
54. The compound according to Embodiment 48, selected from Compound I-49:

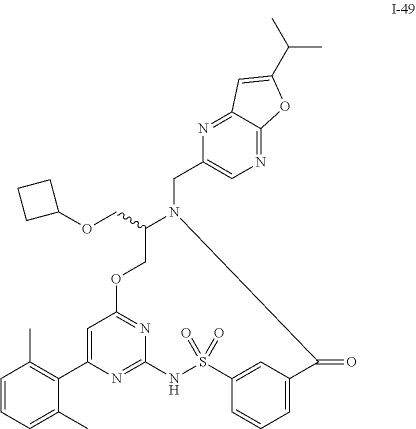 [SMILES: Cc1cccc(C)c1-c1cc2nc(n1)NS(=O)(=O)c1cccc(c1)C(=O)N(Cc1cnc3oc(C(C)C)cc3n1)C(COC1CCC1)CO2]

and tautomers thereof, deuterated derivatives of the compounds and tautomers, and pharmaceutically acceptable salts of any of the foregoing.
55. The compound according to Embodiment 48, selected from Compound I-52:

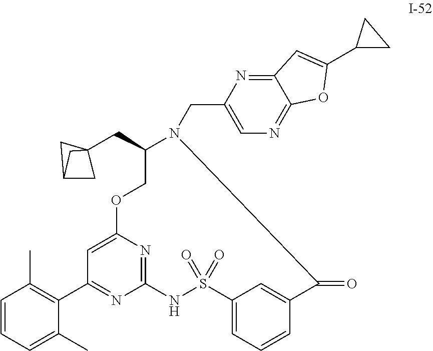

and tautomers thereof, deuterated derivatives of the compounds and tautomers, and pharmaceutically acceptable salts of any of the foregoing.
56. The compound according to Embodiment 48, selected from Compound I-88:

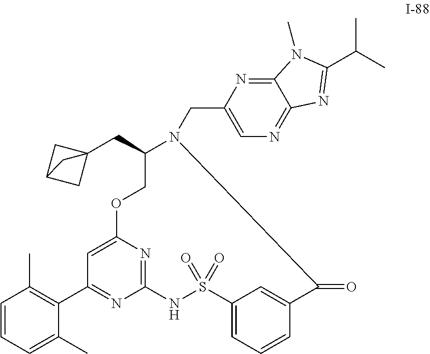 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC13CC(C1)C3)CO2]

and tautomers thereof, deuterated derivatives of the compounds and tautomers, and pharmaceutically acceptable salts of any of the foregoing.
57. The compound according to Embodiment 48, selected from Compound I-96:

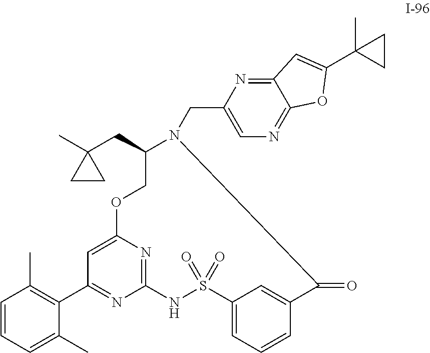 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC1(C)CC1)CO2]

and tautomers thereof, deuterated derivatives of the compounds and tautomers, and pharmaceutically acceptable salts of any of the foregoing.
58. The compound according to Embodiment 48, selected from Compound I-97:

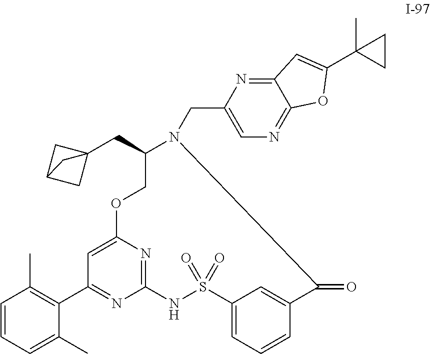 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC13CC(C1)C3)CO2]

and tautomers thereof, deuterated derivatives of the compounds and tautomers, and pharmaceutically acceptable salts of any of the foregoing.
59. The compound according to Embodiment 48, selected from Compound I-98:

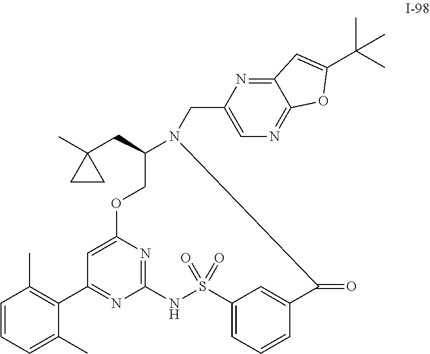 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1(C)CC1)CO2]

and tautomers thereof, deuterated derivatives of the compounds and tautomers, and pharmaceutically acceptable salts of any of the foregoing.
60. The compound according to Embodiment 48, selected from Compound I-99:

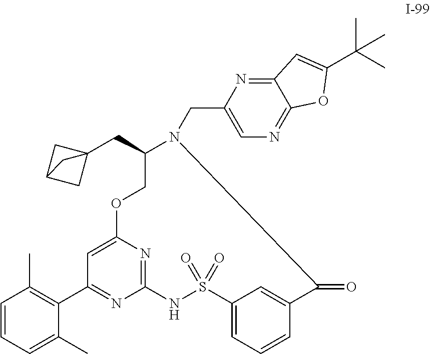 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC13CC(C1)C3)CO2]

and tautomers thereof, deuterated derivatives of the compounds and tautomers, and pharmaceutically acceptable salts of any of the foregoing.
61. The compound according to Embodiment 48, selected from Compound I-139:

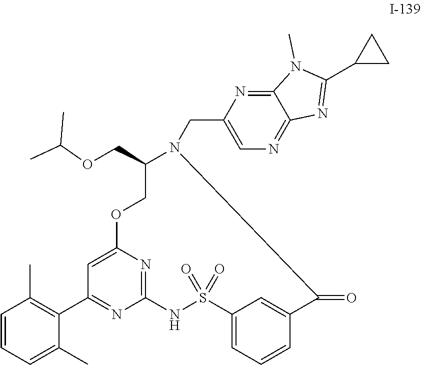 [SMILES: Cc1cccc(C)c1-c1cc2nc(n1)NS(=O)(=O)c1cccc(c1)C(=O)N(Cc1cnc3nc(C4CC4)n(C)c3n1)[C@H](COC(C)C)CO2]

and tautomers thereof, deuterated derivatives of the compounds and tautomers, and pharmaceutically acceptable salts of any of the foregoing.
62. The compound according to Embodiment 48, selected from Compound I-158:

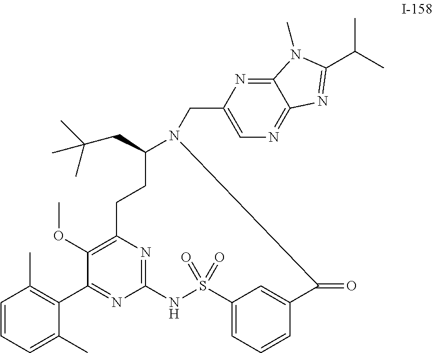 [SMILES: COc1c2nc(nc1-c1c(C)cccc1C)NS(=O)(=O)c1cccc(c1)C(=O)N(Cc1cnc3nc(C(C)C)n(C)c3n1)[C@H](CC(C)(C)C)CC2]

and tautomers thereof, deuterated derivatives of the compounds and tautomers, and pharmaceutically acceptable salts of any of the foregoing.
63. The compound according to Embodiment 48, selected from Compound I-188:

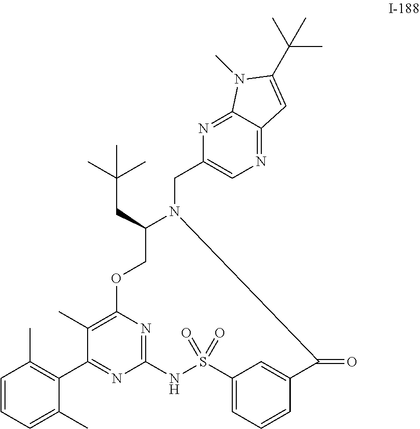 [SMILES: Cc1cccc(C)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2]

and tautomers thereof, deuterated derivatives of the compounds and tautomers, and pharmaceutically acceptable salts of any of the foregoing.
64. The compound according to Embodiment 48, selected from Compound I-206:

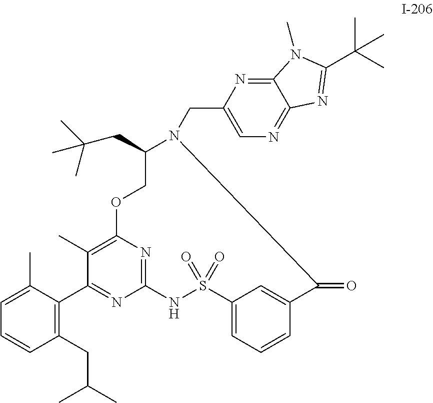 [SMILES: Cc1cccc(CC(C)C)c1-c1nc2nc(c1C)OC[C@@H](CC(C)(C)C)N(Cc1cnc3nc(C(C)(C)C)n(C)c3n1)C(=O)c1cccc(c1)S(=O)(=O)N2]

and tautomers thereof, deuterated derivatives of the compounds and tautomers, and pharmaceutically acceptable salts of any of the foregoing.
65. The compound according to Embodiment 48, selected from Compound I-255:

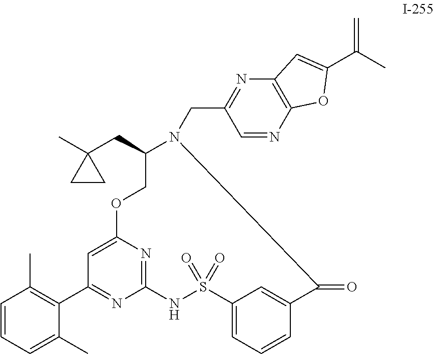 [SMILES: C=C(C)c1cc2nc(CN3C(=O)c4cccc(c4)S(=O)(=O)Nc4nc(cc(-c5c(C)cccc5C)n4)OC[C@H]3CC3(C)CC3)cnc2o1]

and tautomers thereof, deuterated derivatives of the compounds and tautomers, and pharmaceutically acceptable salts of any of the foregoing.
66. The compound according to Embodiment 48, selected from Compound I-256:

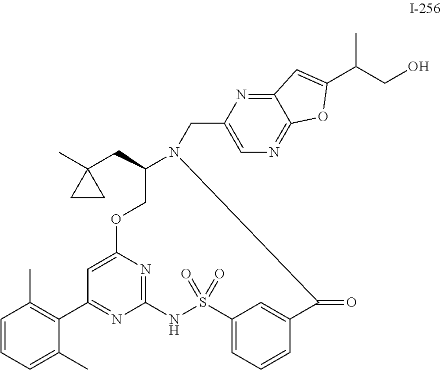 [SMILES: Cc1cccc(C)c1-c1cc2nc(n1)NS(=O)(=O)c1cccc(c1)C(=O)N(Cc1cnc3oc(C(C)CO)cc3n1)[C@H](CC1(C)CC1)CO2]

and tautomers thereof, deuterated derivatives of the compounds and tautomers, and pharmaceutically acceptable salts of any of the foregoing.
67. A compound of Formula II:

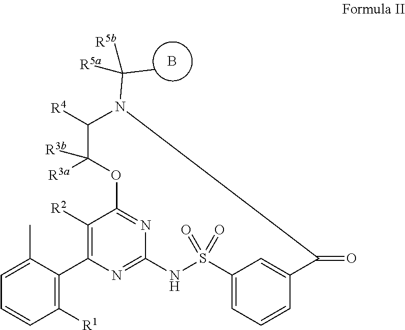

or a tautomer thereof, or a deuterated derivative of the compound or tautomer, or a pharmaceutically acceptable salt of any of the foregoing, wherein:
Ring B is a 6-membered heteroaryl, optionally substituted with 1 to 2 groups independently selected from
halogen
4- to 10-membered heterocyclyl (which may be optionally substituted with 1 to 3 groups independently selected from halogen, oxo, C₁-C₄ alkyl)
N(R^{x})₂, wherein R^{x} is independently selected from hydrogen, C₁-C₄ alkyl, C₃-C₆ cycloalkyl (which may be optionally substituted with a group selected from halogen, C₁-C₄ haloalkyl, and C₁-C₄ alkyl)
C₁-C₄ alkyl (optionally substituted with C₃-C₆ cycloalkyl (which may be further optionally substituted with a group selected from halogen, OH))
   R¹ is selected from: C₃-C₆ cycloalkyl, C₁-C₆ alkoxy, and C₁-C₆ alkyl (which may be optionally substituted with a C₄-C₆ cycloalkyl);
   R² is selected from: hydrogen, halogen, C₁-C₂ alkyl, C₁-C₄ haloalkyl, and C₁-C₂ alkoxy;
   R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
   R⁴ is selected from:
   C₃-C₆ cycloalkyl, which may be optionally substituted with 1 to 3 groups independently selected from halogen, C₁-C₈ haloalkyl, and C₁-C₈ alkyl; and
   C₁-C₉ alkyl, which may be optionally substituted with 1 to 2 groups independently selected from:
      C₃-C₈ cycloalkyl (which may be optionally substituted with 1 to 2 groups selected from C₁-C₈ alkyl, C₁-C₈ alkoxy, halogen, and C₁-C₈ haloalkyl);
      C₁-C₈ haloalkyl;
      phenyl (which may be optionally substituted with a group selected from C₁-C₈ alkyl, C₁-C₈ alkoxy, halogen, and C₁-C₈ haloalkyl);
      C₁-C₈ alkoxy (which may be optionally substituted with a group selected from C₃-C₆ cycloalkyl and halogen);
      4- to 6-membered heterocyclyl (which may be optionally substituted with 1 to 2 groups independently selected from halogen, C₁-C₈ haloalkyl, C₁-C₈ alkyl, and C₁-C₈ alkoxy); and
      silicon (which may be optionally substituted with 1 to 3 groups independently selected from C₁-C₈ alkyl, C₁-C₈ alkoxy, and C₁-C₈ haloalkyl);
   R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
      and wherein the compound of Formula II is selected from Compounds II-1 to II-38 and tautomers thereof, deuterated derivatives of the compounds and tautomers, and pharmaceutically acceptable salts of any of the foregoing.
68. A compound of Formula III:

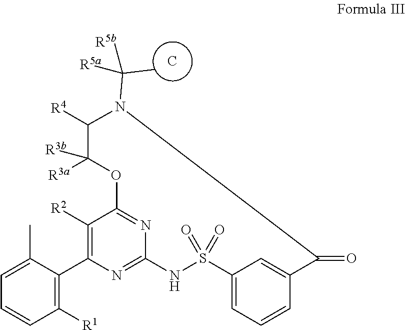

or a tautomer thereof, or a deuterated derivative of the compound or tautomer, or a pharmaceutically acceptable salt of any of the foregoing, wherein:
Ring C is selected from:

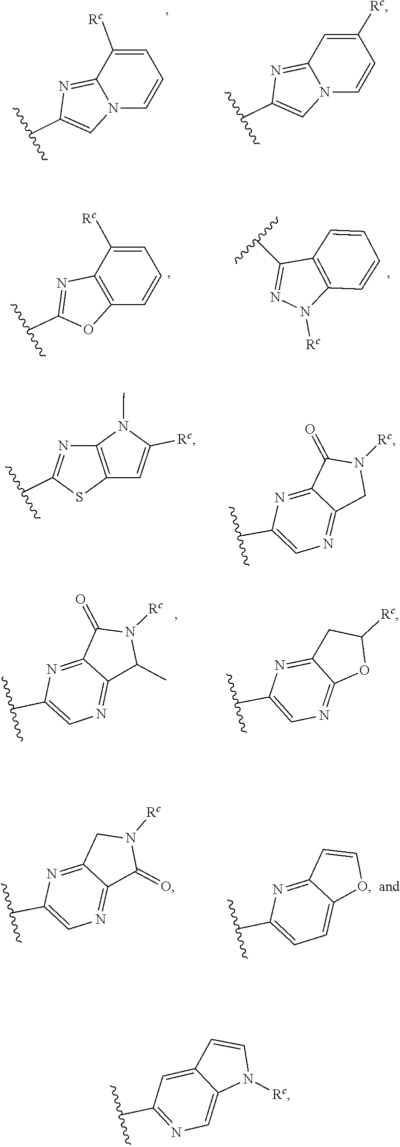

wherein
each R^{c} is independently selected from hydrogen, halogen, cyano, amino, C₁-C₄ alkyl (which may be optionally substituted with a group selected from —OH, halogen, and oxo), and C₃-C₆ alkenyl;
R¹ is selected from: C₃-C₆ cycloalkyl, C₁-C₆ alkoxy, and C₁-C₆ alkyl (which may be optionally substituted with a C₄-C₆ cycloalkyl);
R² is selected from: hydrogen, halogen, C₁-C₂ alkyl, C₁-C₄ haloalkyl, and C₁-C₂ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
   C₃-C₆ cycloalkyl, which may be optionally substituted with 1 to 3 groups independently selected from halogen, C₁-C₈ haloalkyl, and C₁-C₈ alkyl; and
   C₁-C₉ alkyl, which may be optionally substituted with 1 to 2 groups independently selected from:
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl and halogen);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
   and wherein the compound of Formula III is selected from Compounds III-1 to III-25 and tautomers thereof, deuterated derivatives of the compounds and tautomers, and pharmaceutically acceptable salts of any of the foregoing.
69. A compound of Formula IV:

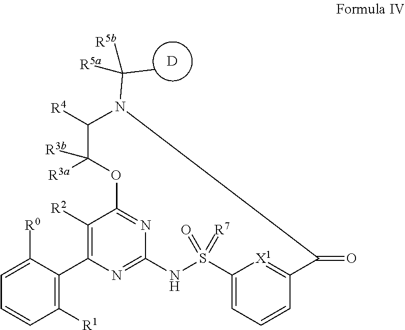

or a tautomer thereof, or a deuterated derivative of the compound or tautomer, or a pharmaceutically acceptable salt of any of the foregoing, wherein:
Ring D is

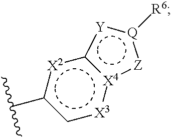

Q is selected from —C— and —N—;
W is selected from —CH—, —C(F)—, —C(CF₃)—, and —N—;
X¹, X², and X³ are each independently selected from —CH— and —N—;
X⁴ is selected from C and N;
Y is selected from —N—, —N(R^{y})—, —C(R^{y})—, and —O—, wherein
   R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CR^{z}—, —O—, —S—, —S(O)—, —S(O)₂—, —N—, and —N^{z}, wherein
   R^{z} is selected from hydrogen, halogen, and C₁-C₈ alkyl (which may be optionally substituted with C₁-C₈ alkoxy;
R⁰ is selected from C₁-C₂ alkyl;
R¹ is selected from: C₃-C₆ cycloalkyl, C₁-C₈ alkoxy, and C₁-C₈ alkyl (which may be optionally substituted with a group selected from C₁-C₈ alkoxy, C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₈ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
   —OH
   C₃-C₈ cycloalkyl (which may be optionally substituted with 1 to 2 groups selected from C₁-C₈ alkyl, C₁-C₈ alkoxy, halogen, and C₁-C₈ haloalkyl);
   C₁-C₈ haloalkyl;
   OC₃-C₇ cycloalkyl (which may be optionally substituted with 1 to 2 groups selected from C₁-C₈ alkyl, C₁-C₈ alkoxy, halogen, and C₁-C₈ haloalkyl);
   phenyl (which may be optionally substituted with a group selected from C₁-C₈ alkyl, C₁-C₈ alkoxy, halogen, and C₁-C₈ haloalkyl);
   C₁-C₈ alkoxy (which may be optionally substituted with a group selected from C₃-C₆ cycloalkyl, and phenyl; or which may be optionally substituted with 1 to 3 halogen atoms);
   4- to 6-membered heterocyclyl (which may be optionally substituted with 1 to 2 groups independently selected from halogen, C₁-C₈ haloalkyl, C₁-C₈ alkyl, and C₁-C₈ alkoxy); and
   silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁶ is selected from hydrogen, cyano, halogen, 4- to 6-membered heterocyclyl, 5- to 6-membered heteroaryl (which may be optionally substituted with 1 to 2 groups selected from C₁-C₈ alkyl), C₃-C₈ cycloalkyl (which may be optionally substituted with a group selected from C₁-C₈ alkyl, C₁-C₈ haloalkyl, and halogen), phenyl, and C₁-C₈ alkyl (which may be optionally substituted with 1 to 2 groups independently selected from C₁-C₈ alkoxy, C₁-C₈ haloalkyl, halogen, oxo, —OH, —NH₂, and —SO₂CH₃); and
R⁷ is selected from O, and NR, wherein
   R is selected from hydrogen and C₁-C₈ alkyl
      with the proviso that wherein the compound of Formula IV is not selected from:

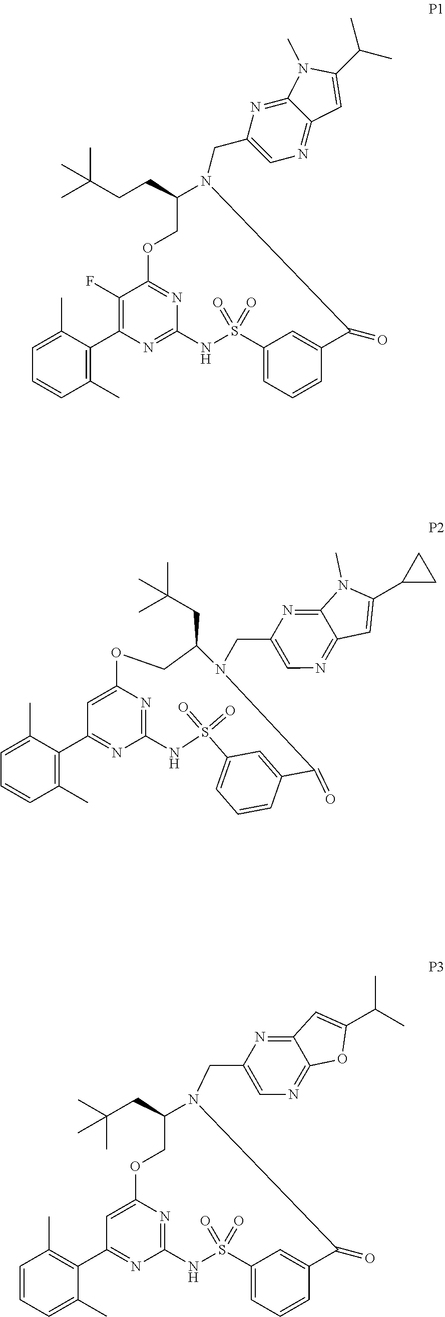 [SMILES: Cc1cccc(C)c1-c1nc2nc(c1F)OC[C@@H](CCC(C)(C)C)N(Cc1cnc3cc(C(C)C)n(C)c3n1)C(=O)c1cccc(c1)S(=O)(=O)N2 ; Cc1cccc(C)c1-c1cc2nc(n1)NS(=O)(=O)c1cccc(c1)C(=O)N(Cc1cnc3cc(C4CC4)n(C)c3n1)[C@H](CC(C)(C)C)CO2 ; Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2]

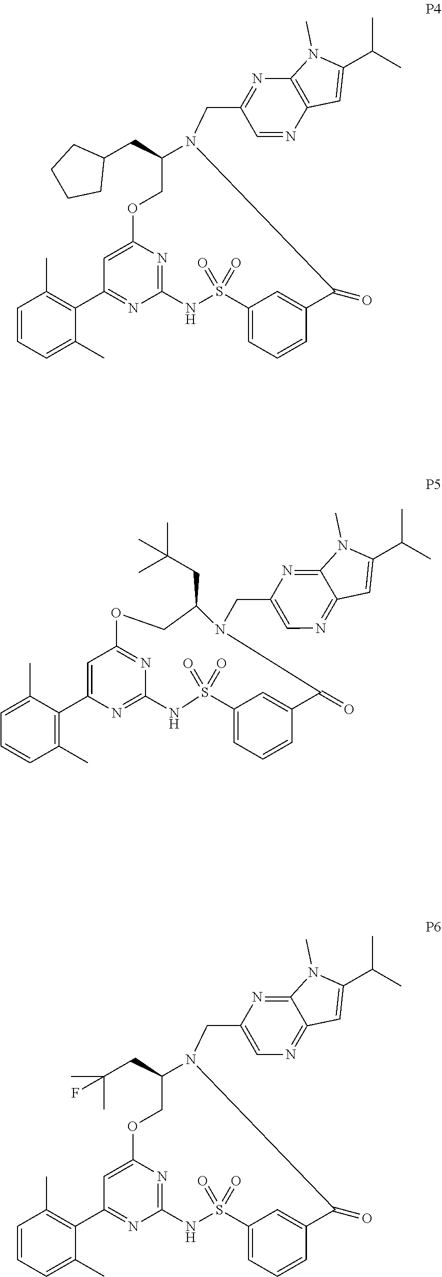 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)F)CO2]

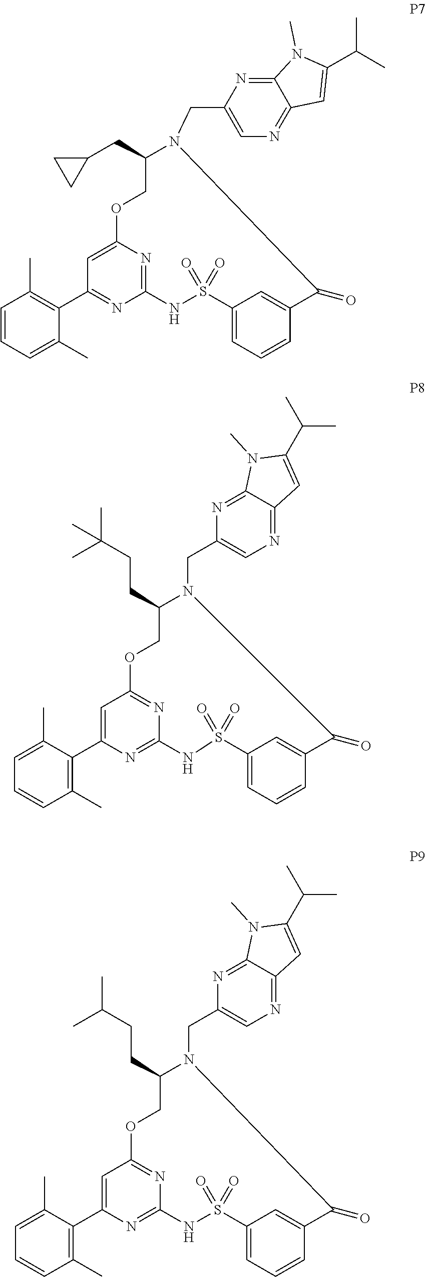

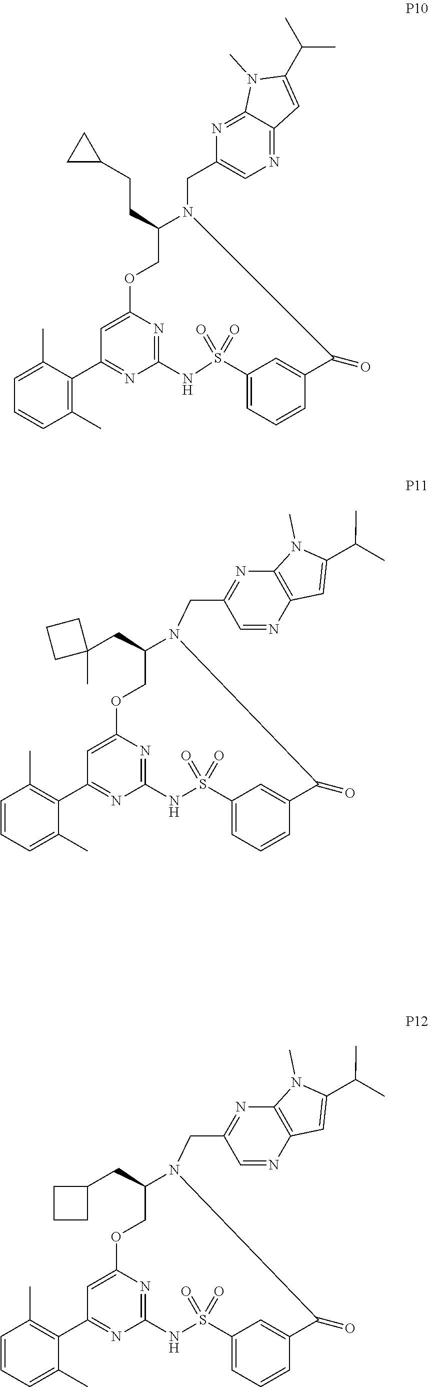 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1(C)CCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1CCC1)CO2]

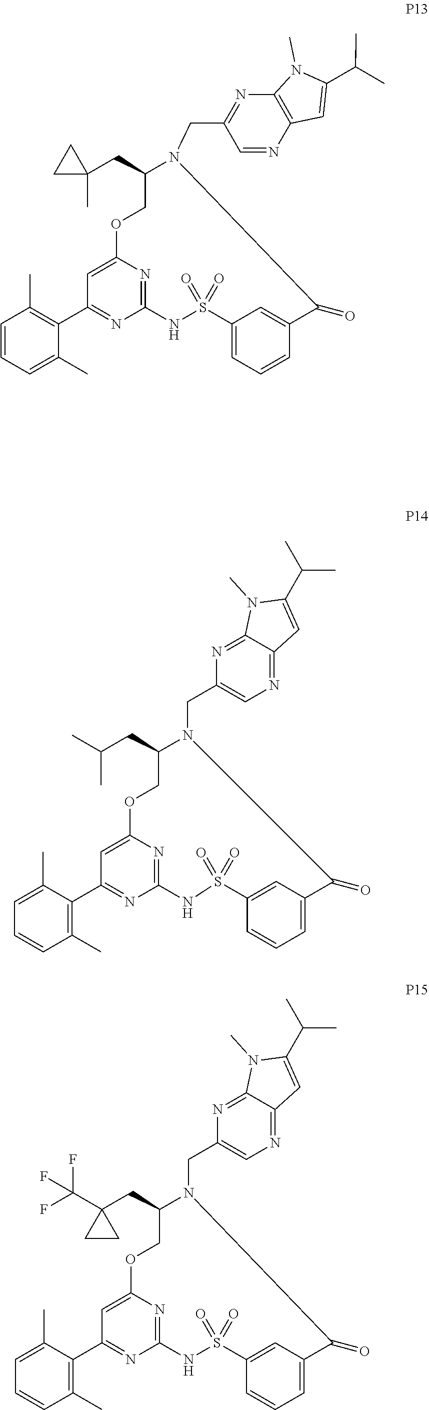 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1(C(F)(F)F)CC1)CO2]

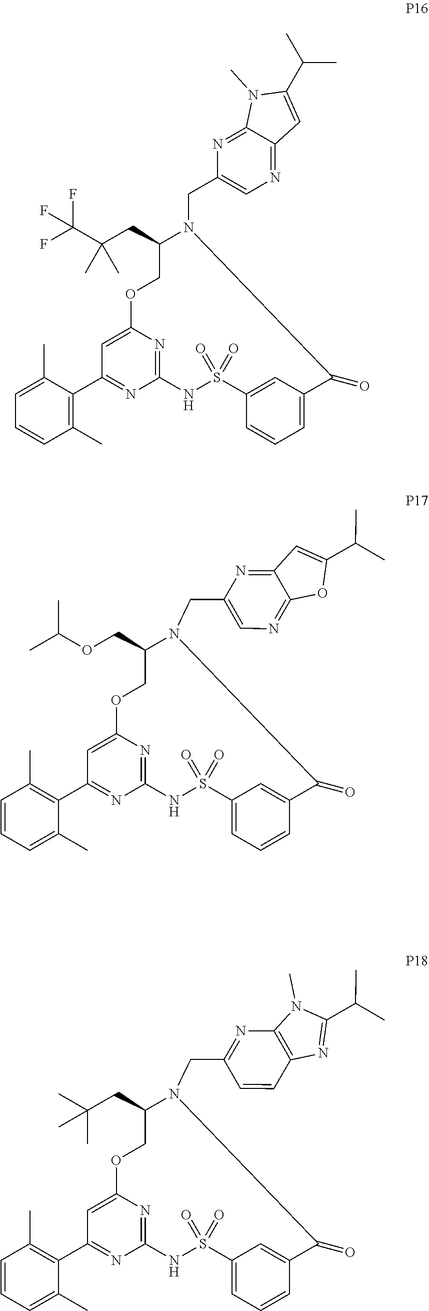 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C(F)(F)F)CO2 ; Cc1cccc(C)c1-c1cc2nc(n1)NS(=O)(=O)c1cccc(c1)C(=O)N(Cc1cnc3oc(C(C)C)cc3n1)[C@H](COC(C)C)CO2 ; Cc1cccc(C)c1-c1cc2nc(n1)NS(=O)(=O)c1cccc(c1)C(=O)N(Cc1ccc3nc(C(C)C)n(C)c3n1)[C@H](CC(C)(C)C)CO2]

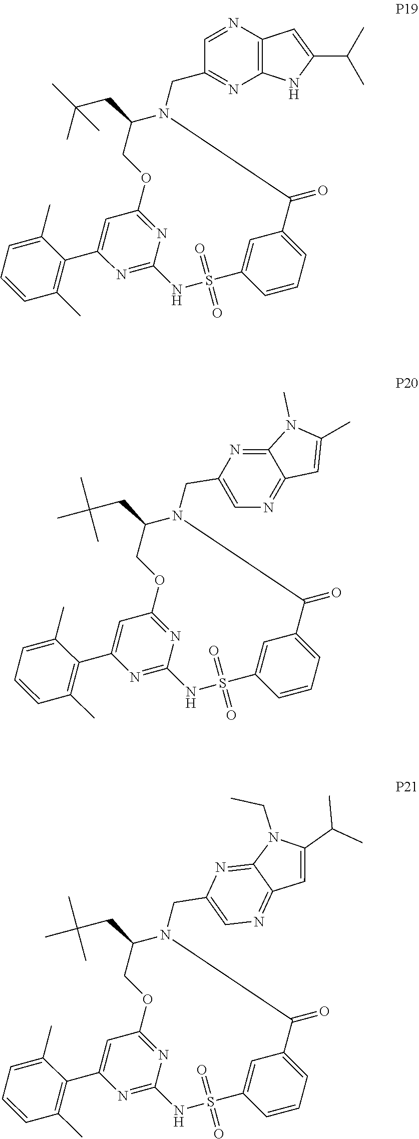 [SMILES: Cc1cccc(C)c1-c1cc2nc(n1)NS(=O)(=O)c1cccc(c1)C(=O)N(Cc1cnc3cc(C(C)C)[nH]c3n1)[C@H](CC(C)(C)C)CO2 ; Cc1cccc(C)c1-c1cc2nc(n1)NS(=O)(=O)c1cccc(c1)C(=O)N(Cc1cnc3cc(C)n(C)c3n1)[C@H](CC(C)(C)C)CO2 ; CCn1c(C(C)C)cc2ncc(CN3C(=O)c4cccc(c4)S(=O)(=O)Nc4nc(cc(-c5c(C)cccc5C)n4)OC[C@H]3CC(C)(C)C)nc21]

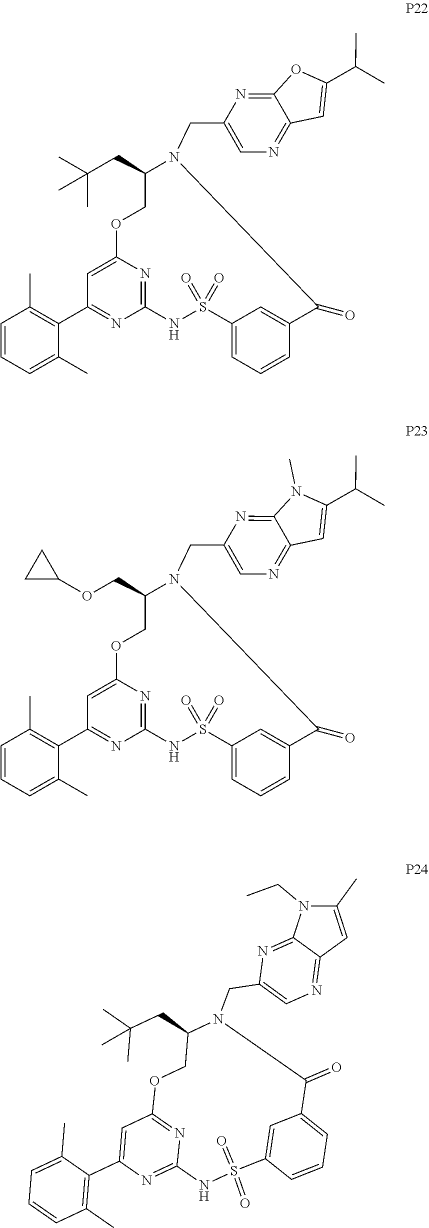 [SMILES: Cc1cccc(C)c1-c1cc2nc(n1)NS(=O)(=O)c1cccc(c1)C(=O)N(Cc1cnc3cc(C(C)C)oc3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](COC1CC1)CO2 ; CCn1c(C)cc2ncc(CN3C(=O)c4cccc(c4)S(=O)(=O)Nc4nc(cc(-c5c(C)cccc5C)n4)OC[C@H]3CC(C)(C)C)nc21]

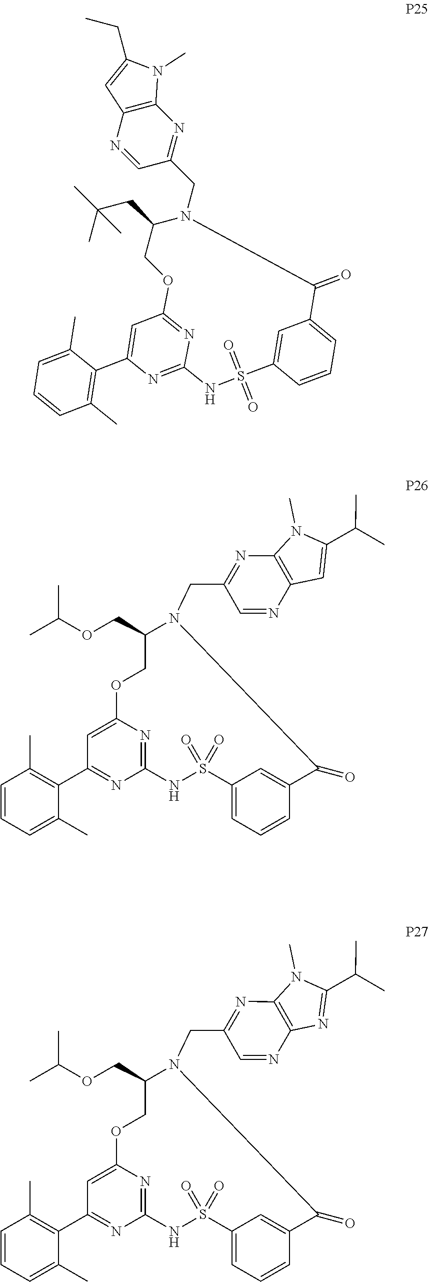 [SMILES: CCc1cc2ncc(CN3C(=O)c4cccc(c4)S(=O)(=O)Nc4nc(cc(-c5c(C)cccc5C)n4)OC[C@H]3CC(C)(C)C)nc2n1C ; Cc1cccc(C)c1-c1cc2nc(n1)NS(=O)(=O)c1cccc(c1)C(=O)N(Cc1cnc3cc(C(C)C)n(C)c3n1)[C@H](COC(C)C)CO2 ; Cc1cccc(C)c1-c1cc2nc(n1)NS(=O)(=O)c1cccc(c1)C(=O)N(Cc1cnc3nc(C(C)C)n(C)c3n1)[C@H](COC(C)C)CO2]

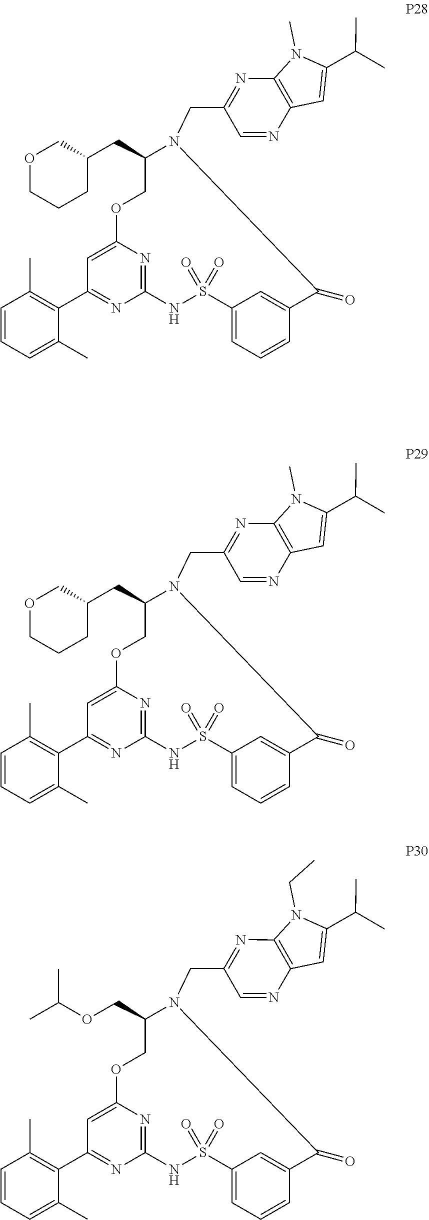 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; CCn1c(C(C)C)cc2ncc(CN3C(=O)c4cccc(c4)S(=O)(=O)Nc4nc(cc(-c5c(C)cccc5C)n4)OC[C@H]3COC(C)C)nc21]

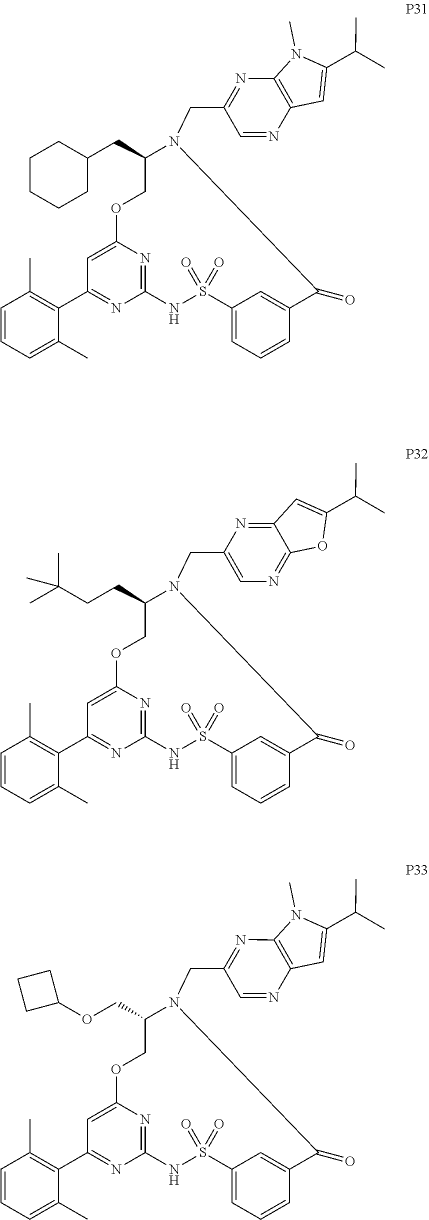 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CCC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2]

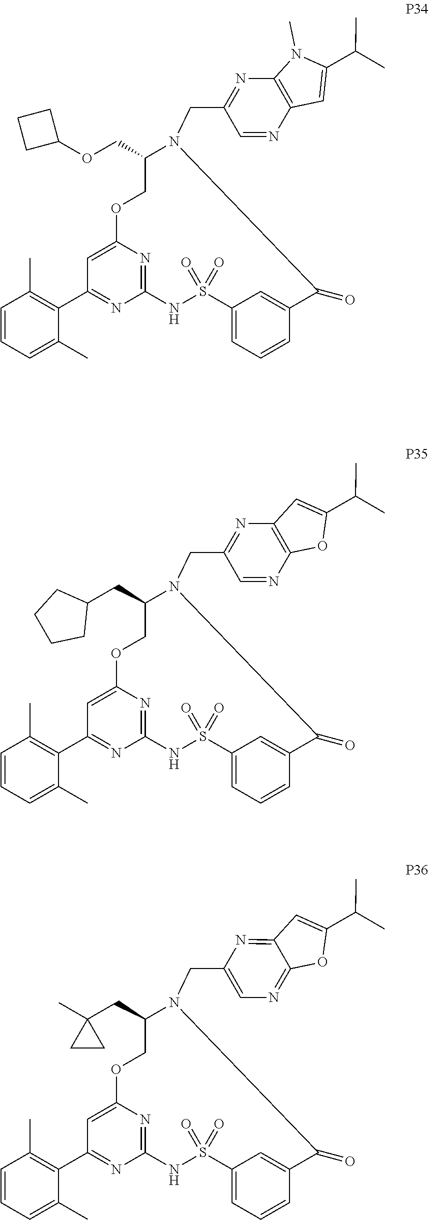 [SMILES: Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1CCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2]

and tautomers, deuterated derivatives, and pharmaceutically acceptable salts thereof.
70. A compound selected from Compounds IV-1 to IV-106, tautomers thereof, deuterated derivatives of the compounds and tautomers, and pharmaceutically acceptable salts of any of the foregoing.
71. A method of treating cystic fibrosis comprising administering a compound, tautomer, deuterated derivative, or pharmaceutically acceptable salt according to any one of Embodiments 1 to 70.
72. The method of Embodiment 71, wherein the compound, tautomer, deuterated derivative, or pharmaceutically acceptable salt according to any one of Embodiments 1 to 70 is administered in combination with at least one additional active pharmaceutical ingredient.
73. The method of Embodiment 72, wherein the at least one additional active pharmaceutical ingredient is selected from mucolytic agents, bronchodilators, antibiotics, anti-infective agents, and anti-inflammatory agents.
74. The method of Embodiment 72, wherein the at least one additional active pharmaceutical ingredient is selected from CFTR potentiators.
75. The method of Embodiment 74, wherein the CFTR potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol and deuterated derivatives and pharmaceutically acceptable salts of any of the foregoing.
76. The method of Embodiment 72, wherein the at least one additional active pharmaceutical ingredient is chosen from CFTR correctors.
77. The method of Embodiment 76, wherein the corrector is selected from lumacaftor, tezacaftor, and deuterated derivatives and pharmaceutically acceptable salts of any of the foregoing.
78. A method of treating cystic fibrosis comprising administering a combination therapy comprising:
   (a) a compound selected from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing; and
   (b) at least one compound selected from tezacaftor, lumacaftor, and deuterated derivatives and pharmaceutically acceptable salts thereof; and/or
   (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]-nonadeca-1(18),2,4,14,16-pentaen-6-ol, and deuterated derivatives and pharmaceutically acceptable salts of any of the foregoing.
79. A compound, tautomer, deuterated derivative, or pharmaceutically acceptable salt according to any one of Embodiments 1 to 70 for use in treating cystic fibrosis.
80. A compound selected from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing for use in treating cystic fibrosis in combination with
   (a) at least one compound selected from tezacaftor, lumacaftor, and deuterated derivatives and pharmaceutically acceptable salts thereof; and/or
   (b) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]-nonadeca-1(18),2,4,14,16-pentaen-6-ol, and deuterated derivatives and pharmaceutically acceptable salts of any of the foregoing.
81. Use of compound, tautomer, deuterated derivative, or pharmaceutically acceptable salt according to any one of Embodiments 1 to 70 in the manufacture of a medicament for treating cystic fibrosis.
82. Use of a compound selected from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing in the manufacture of a medicament for treating cystic fibrosis in combination with
   (a) at least one compound selected from tezacaftor, lumacaftor, and deuterated derivatives and pharmaceutically acceptable salts thereof; and/or
   (b) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]-nonadeca-1(18),2,4,14,16-pentaen-6-ol, and deuterated derivatives and pharmaceutically acceptable salts of any of the foregoing.
83. A pharmaceutical composition comprising a compound, tautomer, deuterated derivative, or pharmaceutically acceptable salt according to any one of Embodiments 1 to 70 and a pharmaceutical carrier.
84. A pharmaceutical composition comprising
   (a) a compound selected from compounds of Formulae I, Ia, Ia(i), Ib, Ib(i), Ic, Ic(i), Id, Id(i), Ie, Ie(i), If, If(i), II, III, and IV, Compounds I-1 to I-265, Compounds II-1 to II-38, Compounds III-1 to III-25, Compounds IV-1 to IV-106, tautomers thereof, deuterated derivatives of those compounds and tautomers, and pharmaceutically acceptable salts of any of the foregoing;
   (b) at least one compound selected from tezacaftor, lumacaftor, and deuterated derivatives and pharmaceutically acceptable salts thereof; and/or
   (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, (6R)-17-amino-12,12-dimethyl-6,15-bis(trifluoromethyl)-19-oxa-3,4,13,18-tetrazatricyclo[12.3.1.12,5]-nonadeca-1(18),2,4,14,16-pentaen-6-ol, and deuterated derivatives and pharmaceutically acceptable salts of any of the foregoing.

### EXAMPLES

### I. Abbreviation List

ACN: Acetonitrile
Boc₂O: Di-tert-butyl dicarbonate
CDMT: 2-Chloro-4,6-dimethoxy-1,3,5-triazine
Cmpd: Compound
DBU: 1,8-Diazabicyclo(5.4.0)undec-7-ene
DCM: Dichloromethane
DI: Deionized
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
EA: Ethyl acetate
diethylether: Diethyl ether
EtOAc: Ethyl acetate
EtOH: Ethanol
GC: Gas chromatography
GCMS: Gas chromatography mass spectrometry
HATU: 1-[Bis(dimethylamino)methylene]-1H-1,2,3-triazolo[4,5-b]pyridinium 3-oxide
hexafluorophosphate
HPLC: High-performance liquid chromatography
IPA: Isopropanol
LAH: Lithium aluminum hydride
LC: Liquid chromatography
LCMS: Liquid chromatography mass spectrometry
LCMS Rt: LCMS retention time
MeCN: Acetonitrile
MeOH: Methanol
MTBE: Methyl tert-butyl ether
MeTHF or 2-MeTHF: 2-Methyltetrahydrofuran
NMP: N-Methyl-2-pyrrolidone
NMM: N-Methylmorpholine
Pd(dppf)Cl₂: [1,1′-Bis(diphenylphosphino)ferrocene]dichloropalladium(II)
RBF: round bottom flask
rt, RT: Room temperature
SFC: Supercritical fluid chromatography
TEA: Triethylamine
TFA: Trifluoroacetic acid
THF: Tetrahydrofuran
TLC: Thin layer chromatography
TMS: Trimethylsilyl
T3P: Propanephosphonic acid anhydride
UPLC: Ultra Performance Liquid Chromatography
Xantphos: 4,5-Bis(diphenylphosphino)-9,9-dimethylxanthene

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. 19F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. 19F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C₁₃/F19/P31) probe (type: 250-SB, s #23055/0020) or on a Varian 500 MHz instrument equipped with a ID PFG, 5 mm, 50-202/500 MHz probe (model/part #99337300).

### II. General UPLC/HPLC Analytical Methods

LC method A: Analytical reverse phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method B: Acquity UPLC BEH C₁₈ column (30×2.1 mm, 1.7 μm particle) made by Waters (pn: 186002349), and a dual gradient run from 1-99% mobile phase B over 1.0 minute. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC method C: Reversed phase UPLC using an Acquity UPLC BEH C₁₈ column (50×2.1 mm, 1.7 μm particle) made by Waters (pn: 186002350), and a dual gradient run from 30-99% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method D: Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC method E: Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC method F: Kinetex Polar C₁₈ 3.0×50 mm 2.6 μm, 6 min, 5-95% ACN in H₂O (0.1% Formic Acid) 1.2 mL/min.

LC method G: Acquity UPLC BEH C₁₈ column (50×2.1 mm, 1.7 μm particle) made by Waters (pn: 186002350), and a dual gradient run from 1-30% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method H: water Cortex 2.7 μ C₁₈(3.0 mm×50 mm), Temp: 55° C.; Flow: 1.2 mL/min; mobile phase: 100% water with 0.1% trifluoroacetic(TFA) acid then 100% acetonitrile with 0.1% TFA acid, grad: 5% to 100% B over 4 min, with stay at 100% B for 0.5 min, equilibration to 5% B over 1.5 min.

LC method I: UPLC Luna C₁₈(2) 50×3 mm 3 μm. run: 2.5 min. Mobile phase: Initial 95% H₂O 0.1% FA/5% MeCN 0.1% FA, linear grad to 95% MeCN 0.1% FA over 1.3 min, hold 1.2 min 95% CH₃CN 0.1% FA,.T: 45 C, Flow: 1.5 mL/min.

LC method J: UPLC SunFire C₁₈ 75×4.6 mm 3.5 μm, run: 6 min. Mobile phase conditions: Initial 95% H₂O+0.1% FA/5% CH₃CN+0.1% FA, linear gradient to 95% CH₃CN for 4 min, hold for 2 min at 95% CH₃CN. T: 45° C., Flow: 1.5 mL/min.

LC method K: XBridge C₁₈ 4.6×75 mm, 5 m, Initial Gradient at 95% NH₄HCO₃/5% MeCN 6 min run with 1 min equilibration gradient 0 to 3 min at 95% MeCN and hold for 3 minutes. Flow 1.5 mL/min.

LC method L: Luna C₁₈ 3.0×50 mm 3.0 μM, Temp: 45° C., Flow: 2.0 mL/min, Run Time: 3 minutes. Mobile Phase: Initial 95% H₂O (0.1% Formic Acid) and 5% CH₃CN (0.1% FA) linear gradient to 95% CH₃CN (0.1% FA) for 2.0 min then hold at 95% CH₃CN (0.1% FA) for 1.0 min

LC method M: Analytical reverse phase UPLC-MS using an Acquity UPLC-MS BEH C₁₈ column (50×2.1 mm, 1.7 μm particle size) made by Waters (pn: 186002350), and a dual gradient run from 1% to 99% mobile phase B over 5.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

### IV. Synthesis of Common Intermediates

### Example 1: Preparation of 3-[[4-chloro-6-(2,6-dimethylphenyl)pyrimidin-2-yl]sulfamoyl]benzoic acid

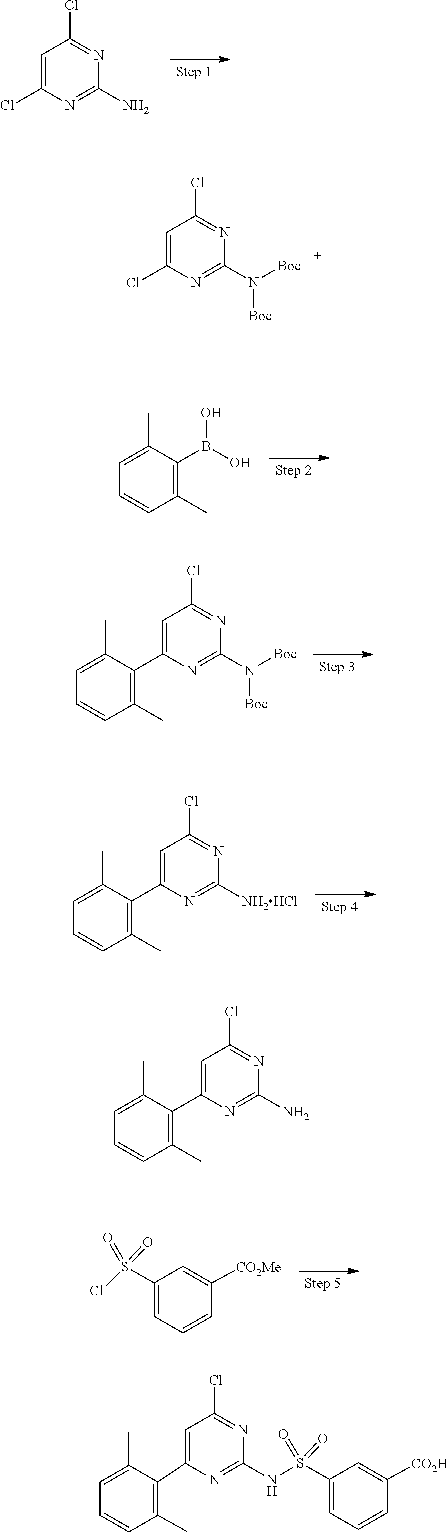

### Step 1: tert-Butyl N-tert-butoxycarbonyl-N-(4,6-dichloropyrimidin-2-yl)carbamate

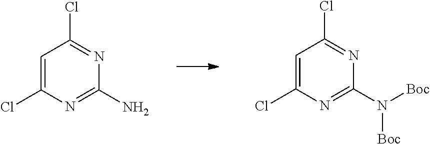 [SMILES: Nc1nc(Cl)cc(Cl)n1 ; CC(C)(C)OC(=O)N(C(=O)OC(C)(C)C)c1nc(Cl)cc(Cl)n1]

To a solution of 4,6-dichloropyrimidin-2-amine (300 g, 1.829 mol) in DCM (2.1 L) was added (BOC)₂O (838 g, 3.840 mol) followed by DMAP (5.6 g, 45.84 mmol). The mixture was stirred at ambient temperature for 6 h. Additional DMAP (5.6 g, 45.84 mmol) was added and the reaction was continued to stir at ambient temperature for 24 h. The mixture was diluted with water (2.1 L) and the organic phase separated. The organic phase was washed with water (2.1 L), 2.1 L of brine, dried over magnesium sulfate, filtered over Celite and concentrated in vacuo affording a light orange oil which had a silt in the slurry. The mixture was diluted with ˜500 mL of heptane and filtered using an M filter. The precipitate (SM) was washed with 250 mL of heptane. The filtrate was concentrated in vacuo affording a thick orange oil which was seeded with solid from a previous experiment and crystallized on standing, affording a light orange hard solid. tert-butyl N-tert-butoxycarbonyl-N-(4,6-dichloropyrimidin-2-yl)carbamate (645 g, 97%). ¹H NMR (400 MHz, DMSO-d6) δ 8.07 (s, 1H), 1.44 (s, 18H). ESI-MS m/z calc. 363.07526, found 364.1 (M+1)⁺; Retention time: 2.12 minutes (LC method A).

### Step 2: tert-Butyl N-tert-butoxycarbonyl-N-[4-chloro-6-(2,6-dimethylphenyl)pyrimidin-2-yl]carbamate

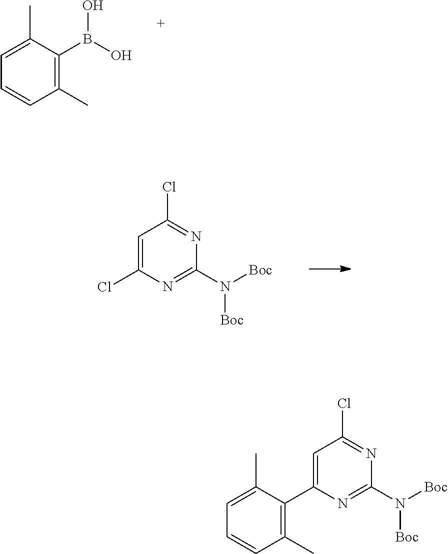 [SMILES: Cc1cccc(C)c1B(O)O ; CC(C)(C)OC(=O)N(C(=O)OC(C)(C)C)c1nc(Cl)cc(Cl)n1 ; Cc1cccc(C)c1-c1cc(Cl)nc(N(C(=O)OC(C)(C)C)C(=O)OC(C)(C)C)n1]

All solvents were degassed prior to use. To a slurry of tert-butyl N-tert-butoxycarbonyl-N-(4,6-dichloropyrimidin-2-yl)carbamate (88 g, 241.6 mmol), (2,6-dimethylphenyl)boronic acid (approximately 36.24 g, 241.6 mmol) and Cs₂CO₃ (approximately 196.8 g, 604.0 mmol) in DME (704 mL) and water (176 mL) were added. Pd(dppf)Cl₂ (approximately 8.839 g, 12.08 mmol) was added and the mixture was vigorously stirred under nitrogen at 80° C. (reflux) for 1 h (no SM remained). The reaction was cooled to ambient temperature and diluted with water (704 mL). The aqueous phase was separated and extracted with EtOAc (704 mL). The organic phase was washed with 700 mL of brine, dried over magnesium sulfate, filtered and concentrated in vacuo. The crude product was chromatographed on a 1500 g silica gel column eluting with 0-30% EtOAc/hexanes. The product fractions (eluted at 15% EtOAc) were combined and concentrated in vacuo affording the product as a clear oil which crystallized on standing. tert-butyl N-tert-butoxycarbonyl-N-[4-chloro-6-(2,6-dimethylphenyl)pyrimidin-2-yl]carbamate (81.3 g, 78%). ¹H NMR (400 MHz, DMSO-d6) δ 7.88 (s, 1H), 7.30 (dd, J 8.2, 7.0 Hz, 1H), 7.21-7.16 (m, 2H), 2.03 (s, 6H), 1.38 (s, 18H). ESI-MS m/z calc. 433.17682, found 434.1 (M+1)⁺; Retention time: 2.32 minutes (LC method A).

### Step 3: 4-Chloro-6-(2,6-dimethylphenyl)pyrimidin-2-amine (hydrochloride salt

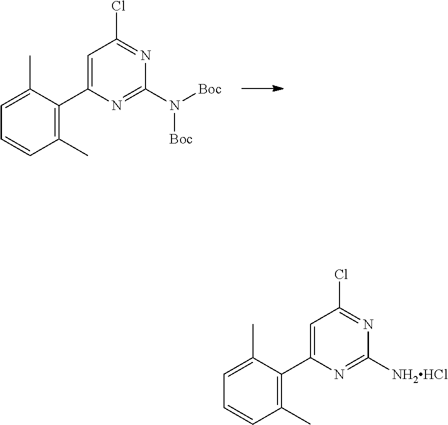

tert-Butyl N-tert-butoxycarbonyl-N-[4-chloro-6-(2,6-dimethylphenyl)pyrimidin-2-yl]carbamate (514.8 g, 915.9 mmol) was dissolved in dichloromethane (4 L). Hydrogen chloride in p-dioxane (1 μL, 4 mol) was added and the mixture was stirred overnight at room temperature. The resulting precipitate was collected by vacuum filtration and dried in vacuo to obtain 4-chloro-6-(2,6-dimethylphenyl)pyrimidin-2-amine hydrochloride (213.5 g, 64%) as a white solid (213.5 g, 82%). ¹H NMR (250 MHz, DMSO-d6) δ 7.45-6.91 (m, 3H), 6.73 (s, 1H), 2.08 (s, 6H). ESI-MS m/z calc. 233.072, found 234.1 (M+1)⁺; Retention time: 2.1 minutes (LC Method C).

### Step 4: 4-Chloro-6-(2,6-dimethylphenyl)pyrimidin-2-amine

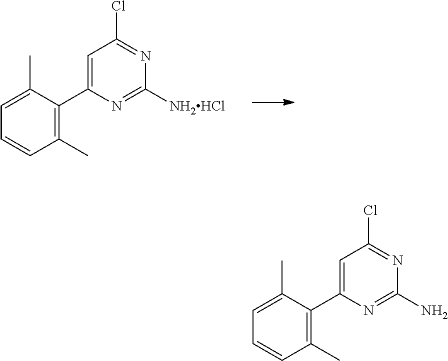

4-Chloro-6-(2,6-dimethylphenyl)pyrimidin-2-amine (hydrochloride salt) (166 g, 614.5 mmol) and 4-chloro-6-(2,6-dimethylphenyl)pyrimidin-2-amine (hydrochloride salt) (30 g, 111.0 mmol) were suspended in DCM (2.5 L), treated with NaOH (725 mL of 1 M, 725.0 mmol) and stirred at room temperature for 1 hour. The mixture was transferred into a separatory funnel and left standing over night. The DCM phase was separated and the aqueous phase with insoluble material was extracted twice more with DCM (2×500 mL). The combined brown DCM phases were stirred over magnesium sulfate and charcoal for 1 hour, filtered and the yellow solution concentrated to a volume of ˜500 mL. The solution was diluted with heptane (750 mL) and DCM was removed under reduced pressure at 60° C. to give a cream suspension. It was stirred at room temperature for 1 hour, filtered, washed with cold heptane and dried to give 4-chloro-6-(2,6-dimethylphenyl)pyrimidin-2-amine (157 g, 91%) as a cream solid. H NMR (400 MHz, DMSO-d6) δ 7.28-7.14 (m, 3H), 7.10 (d, J 7.5 Hz, 2H), 6.63 (s, 1H), 2.06 (s, 6H). ESI-MS m/z calc. 233.07198, found 234.0 (M+1)⁺; Retention time: 1.45 minutes (LC method A).

### Step 5: 3-[[4-Chloro-6-(2,6-dimethylphenyl)pyrimidin-2-yl]sulfamoyl]benzoic acid

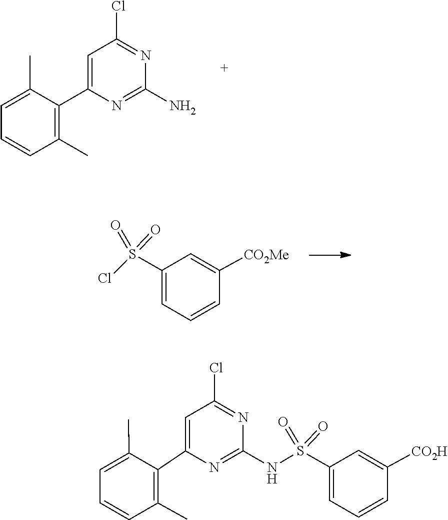 [SMILES: Cc1cccc(C)c1-c1cc(Cl)nc(N)n1 ; COC(=O)c1cccc(S(=O)(=O)Cl)c1 ; Cc1cccc(C)c1-c1cc(Cl)nc(NS(=O)(=O)c2cccc(C(=O)O)c2)n1]

4-Chloro-6-(2,6-dimethylphenyl)pyrimidin-2-amine (235 g, 985.5 mmol) was dissolved in MeTHF (2.3 L) and cooled in an ice bath under stirring and nitrogen. To the cold solution methyl 3-chlorosulfonylbenzoate (347 g, 1.479 mol) was added in one portion (seems slightly endothermic) and to the cold pale-yellow solution a solution of 2-methyl-butan-2-ol (Lithium salt) (875 mL of 3.1 M, 2.712 mol) (in heptane) was added dropwise over 1.25 hour (exothermic, internal temperature from 0 to 10° C.). The ice bath was removed and the greenish solution was stirred for 4 hours at room temperature. To the greenish solution cold HCl (2 L of 1.5 M, 3.000 mol) was added, the phases separated and the organic phase was washed once with water (1 L) and once with brine (500 mL). The aqueous phases were back extracted once with MeTHF (350 mL) and the organic phases were combined. This yellow MeTHF solution of methyl 3-[[4-chloro-6-(2,6-dimethylphenyl)pyrimidin-2-yl]sulfamoyl]benzoate (ESI-MS m/z calc. 431.07065, found 432.0 (M+1)⁺; Retention time: 1.81 minutes) was treated with NaOH (2.3 L of 2 M, 4.600 mol) and stirred at room temperature for 1 hour. The phases were separated and the NaOH phase was washed twice with MeTHF (2×500 mL) and the combined organic phases were extracted once with 2M NaOH (1×250 mL). The combined NaOH phases were combined, stirred in an ice bath and slowly acidified by addition of HCl (416 mL of 36% w/w, 4.929 mol) while keeping the internal temperature between 1° and 20° C. At the end of the addition (pH ˜5-6) the final pH was adjusted to 2-3 by addition of solid citric acid. The formed yellow tacky suspension was stirred at room temperature overnight to give a cream crisp suspension. The solid was collected by filtration, washed with plenty of water and sucked dry for 3 hours. The solid was dried under reduced pressure with a nitrogen leak at 45-50° C. for 120 hours 3-[[4-chloro-6-(2,6-dimethylphenyl)pyrimidin-2-yl]sulfamoyl]benzoic acid (395 g, 96%) was isolated as an off-white solid. ¹H NMR (400 MHz, DMSO-d6) δ 13.44 (s, 1H), 12.46 (s, 1H), 8.48-8.39 (m, 1H), 8.25-8.15 (m, 1H), 8.15-8.08 (m, 1H), 7.68 (t, J 7.8 Hz, 1H), 7.31 (s, 1H), 7.28-7.18 (m, 1H), 7.10 (d, J 7.6 Hz, 2H), 1.84 (s, 6H). ESI-MS m/z calc. 417.055, found 418.0 (M+1)⁺; Retention time: 1.56 minutes. (LC method A).

### Example 2: Preparation of [[4-[(2R)-2-(tert-butoxycarbonylamino)-4-methyl-pentoxy]-6-(2,6-dimethylphenyl)pyrimidin-2-yl]sulfamoyl]benzoic acid

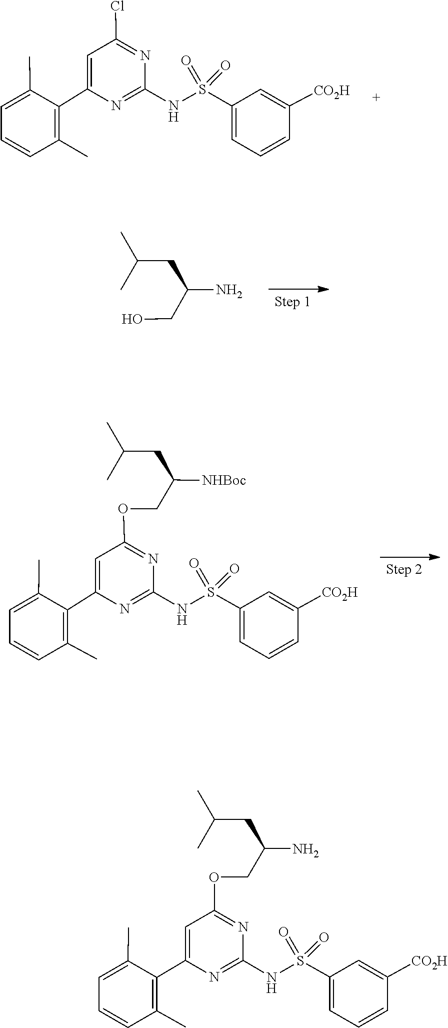

### Step 1: 3-[[4-[(2R)-2-(tert-Butoxycarbonylamino)-4-methyl-pentoxy]-6-(2,6-dimethylphenyl)pyrimidin-2-yl]sulfamoyl]benzoic acid

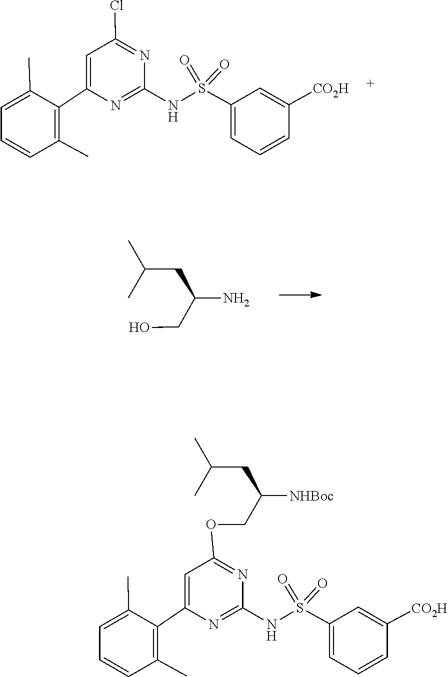

To a stirring solution of (2R)-2-amino-4-methyl-pentan-1-ol (12.419 g, 105.97 mmol) in anhydrous THE (200 mL) at room temperature under nitrogen was added sodium tert-butoxide (15.276 g, 158.95 mmol). The reaction mixture was stirred for 10 minutes and 3-[[4-chloro-6-(2,6-dimethylphenyl)pyrimidin-2-yl]sulfamoyl]benzoic acid (22.14 g, 52.983 mmol) was added. The reaction mixture was placed on a water bath preheated to 60° C. and stirred for 20 minutes. After cooling to room temperature, di-tert-butyl dicarbonate (69.381 g, 317.90 mmol) was added and the reaction mixture was stirred for 3 hours. The reaction was quenched with saturated aqueous ammonium chloride (150 mL). Volatiles were removed under vacuum and the aqueous layer was acidified to pH ˜3 with 10% aqueous citric acid. The product was extracted with ethyl acetate (3×200 mL). The combined organic layers were washed with brine (80 mL), dried over anhydrous sodium sulfate and concentrated to a residual volume of ˜250 mL. The product was precipitated out into excess hexanes (750 mL) and collected by vacuum filtration. The obtained white solid was re-purified by silica gel chromatography using 0-40% acetone (0.15% acetic acid buffer) gradient in hexanes (0.15% acetic acid buffer) to afford 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 61%) as a white solid. ESI-MS m/z calc. 598.2461, found 599.4 (M+1)⁺; Retention time: 5.85 minutes (LC Method D).

### Step 2: 3-[[4-[(2R)-2-Amino-4-methyl-pentoxy]-6-(2,6-dimethylphenyl)pyrimidin-2-yl]sulfamoyl]benzoic acid (hydrochloride salt

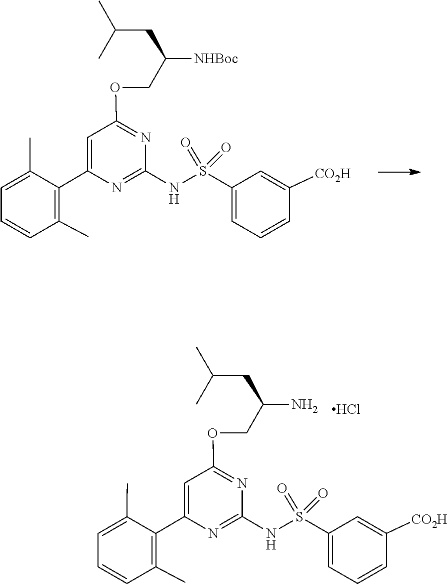 [SMILES: Cc1cccc(C)c1-c1cc(OC[C@@H](CC(C)C)NC(=O)OC(C)(C)C)nc(NS(=O)(=O)c2cccc(C(=O)O)c2)n1 ; Cc1cccc(C)c1-c1cc(OC[C@H](N)CC(C)C)nc(NS(=O)(=O)c2cccc(C(=O)O)c2)n1 ; Cl]

To a stirring solution of 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 34.624 mmol) in DCM (200 mL) at room temperature was added HCl (87 mL of 4 M solution in 1,4-dioxane, 346.24 mmol). The reaction mixture was stirred for 2 hours. Volatiles were removed under vacuum and the obtained solid was triturated with diethyl ether (150 mL). After removal of the volatiles, the product was dried under vacuum to afford 3-[[4-[(2R)-2-amino-4-methyl-pentoxy]-6-(2,6-dimethylphenyl)pyrimidin-2-yl]sulfamoyl]benzoic acid (hydrochloride salt) (19.68 g, 100%) as a white solid. H NMR (250 MHz, DMSO-d6) δ 8.56-8.27 (m, 4H), 8.14 (t, J 6.8 Hz, 2H), 7.70 (t, J 7.8 Hz, 1H), 7.34-7.18 (m, 1H), 7.17-7.02 (m, 2H), 6.31 (s, 1H), 4.42-4.23 (m, 1H), 4.23-4.06 (m, 1H), 3.5-3.4 (m, 1H, overlapped with water), 2.01 (s, 6H), 1.82-1.31 (m, 3H), 1.02-0.78 (m, 6H). ESI-MS m/z calc. 498.1937, found 499.3 (M+1)⁺; Retention time: 1.63 minutes (LC Method E).

### Example 3: Preparation of 3-[[4-[(2R)-2-amino-4,4-dimethyl-pentoxy]-6-(2,6-dimethylphenyl)pyrimidin-2-yl]sulfamoyl]benzoic acid

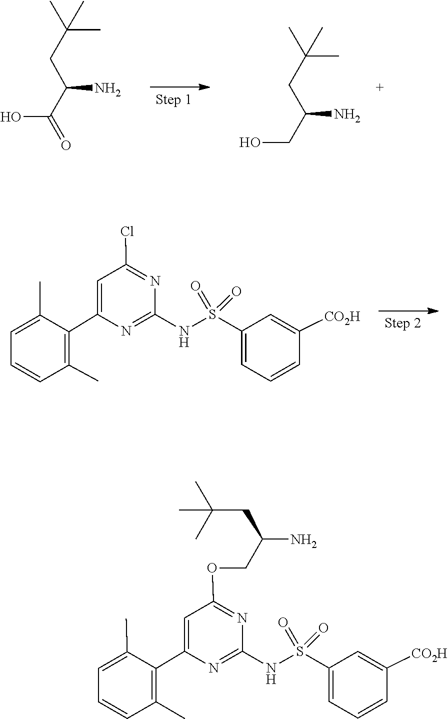

### Step 1: (2R)-2-Amino-4,4-dimethyl-pentan-1-ol

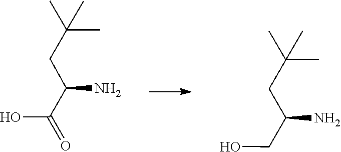

To a solution of (2R)-2-amino-4,4-dimethyl-pentanoic acid (15 g, 103.3 mmol) in THE (150 mL) at 0° C. was added borane-THF (260 mL of 1 M, 260.0 mmol) dropwise keeping the reaction temperature <10° C. The addition took approximately 30 min. The mixture was allowed to warm to ambient temperature and stirred for 22 h. The reaction was quenched with the slow addition of methanol (80 mL, 1.975 mol) and the solvent was removed in vacuo. The residue was co-evaporated 3× with methanol (200 mL, 4.937 mol) The crude residue was diluted with HCl (200 mL of 1 M, 200.0 mmol) and washed with 200 mL of MTBE. The aqueous phase was evaporated to remove residual organic solvent. The water was further removed in vacuo affording an off-white solid. The solid was further dried using an acetonitrile azeotrope. The solid was slurried in 200 mL of ACN and the precipitate collected using a M frit. The solid was air dried for 1 h, then in vacuo at 45° C. for 20 h to give (2R)-2-amino-4,4-dimethyl-pentan-1-ol (hydrochloride salt) (14.73 g, 85%). ¹H NMR (400 MHz, DMSO-d6) δ 7.80 (s, 3H), 5.36 (t, J 5.1 Hz, 1H), 3.59 (dt, J 11.7, 4.1 Hz, 1H), 3.42-3.34 (m, 1H), 3.10 (dq, J 7.7, 3.8 Hz, 1H), 1.46 (dd, J=14.5, 7.1 Hz, 1H), 1.33 (dd, J=14.5, 3.5 Hz, 1H), 0.91 (s, 9H). ESI-MS m/z calc. 131.13101, found 132.1 (M+1)⁺; Retention time: 0.51 minutes (LC method A).

### Step 2: 3-[[4-[(2R)-2-Amino-4,4-dimethyl-pentoxy]-6-(2,6-dimethylphenyl)pyrimidin-2-yl]sulfamoyl]benzoic acid

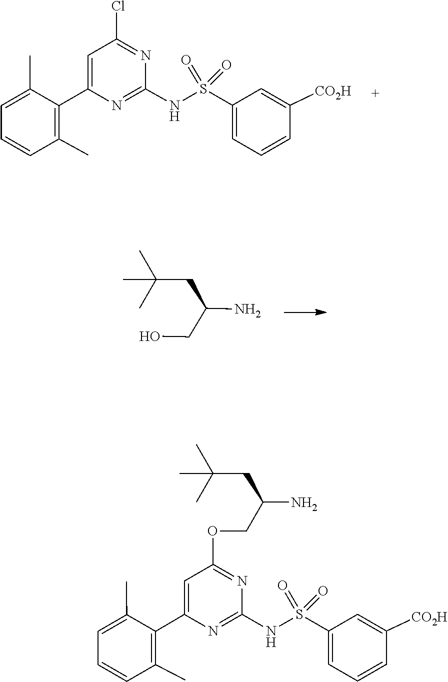

3-[[4-Chloro-6-(2,6-dimethylphenyl)pyrimidin-2-yl]sulfamoyl]benzoic acid (20 g, 47.862 mmol) was suspended in a mixture of 2-methyltetrahydrofuran (80 mL) and DMF (20 mL) and the solution was cooled to −5° C. Sodium tert-butoxide (23 g, 239.33 mmol) was then dissolved in 2-methyltetrahydrofuran (100 mL), cooled to 5° C. and added over 10 minutes, followed by (2R)-2-amino-4,4-dimethyl-pentan-1-ol (hydrochloride salt) (8.02 g, 47.830 mmol) the reaction was then warmed to 10° C. and stirred for 4 hours. It was then cooled to 0° C. and quenched by adding an aqueous solution of hydrochloric acid (2 M, 200 mL) over 10 minutes. The phases were separated, and the aqueous phase extracted with 2-methyltetrahydrofuran (200 mL). The organic phases were combined and washed with an aqueous solution of sodium chloride (15% w/w, 2×200 mL), dried over sodium sulfate (60 g), filtered and evaporated to dryness. The solid was then triturated using ethyl acetate (200 mL) for 16 hours, filtered, washed with ethyl acetate and dried in a vacuum oven at 50° C. for 20 hours to give 3-[[4-[(2R)-2-amino-4,4-dimethyl-pentoxy]-6-(2,6-dimethylphenyl)pyrimidin-2-yl]sulfamoyl]benzoic acid (hydrochloride salt) (22.29 g, 80%). ¹H NMR (400 MHz, DMSO-d6) δ 13.26 (br. s., 2H), 8.45 (t, J 1.6 Hz, 1H), 8.28-8.06 (m, 5H), 7.69 (t, J 7.8 Hz, 1H), 7.31-7.21 (m, 1H), 7.13 (d, J 7.6 Hz, 2H), 6.29 (br. s., 1H), 4.30 (dd, J 11.7, 2.7 Hz, 1H), 4.10 (dd, J 11.5, 7.1 Hz, 1H), 3.56 (br. s., 1H), 2.13-1.90 (s, 6H), 1.62-1.47 (m, 2H), 0.94 (s, 9H). ESI-MS m/z calc. 512.20935, found 513.0 (M+1)⁺; Retention time: 2.334 minutes; LC method F.

### Example 4: Preparation of 3-[[4-[(2R)-2-amino-5,5,5-trifluoro-4,4-dimethyl-pentoxy]-6-(2,6-dimethylphenyl)pyrimidin-2-yl]sulfamoyl]benzoic acid

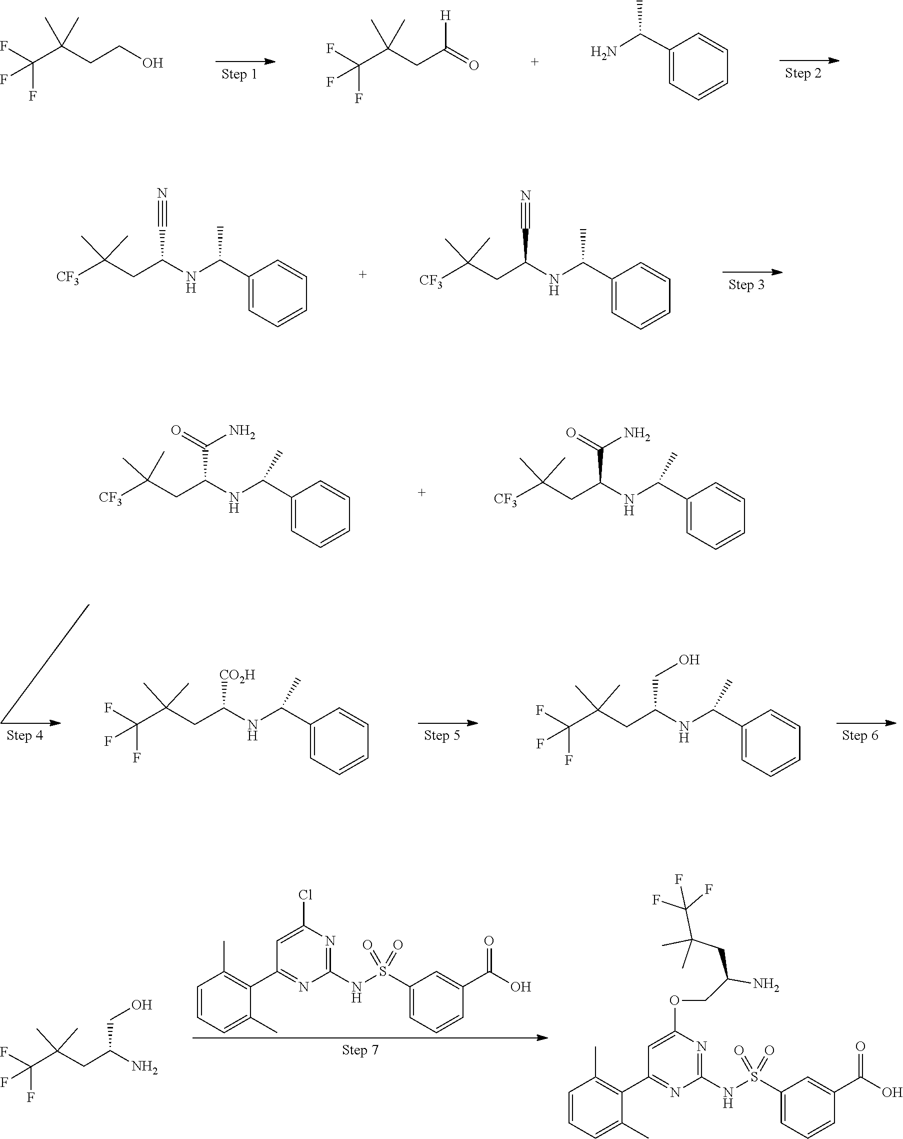

### Step 1: 4,4,4-Trifluoro-3,3-dimethyl-butanal

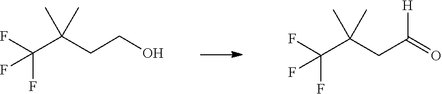

A 1 L three-neck flask was charged with 4,4,4-trifluoro-3,3-dimethyl-butan-1-ol (8.987 g, 57.555 mmol), DCM (63 mL), water (63 mL), NaBr (544 mg, 5.2870 mmol), sodium bicarbonate (12.32 g, 146.66 mmol) and TEMPO (92 mg, 0.5888 mmol). The mixture was cooled with ice-water bath. A aqueous solution of NaOCl (47 mL of 1.31 M, 61.570 mmol) was added dropwise over 2 h at 2.5-4.4° C. After the addition, the mixture was stirred for 10 min. The two layers was separated. The aqueous phase was extracted with DCM (2×15 mL). The combined organic layers were dried with sodium sulfate and filtered to give 113.7 g (about 80 mL) of crude product in DCM, which was used directly the next step. ¹H NMR (300 MHz, CDCl₃) δ 9.82-9.78 (m, 1H), 2.54 (d, J=2.6 Hz, 2H), 1.28 (s, 6H). ¹⁹F NMR (282 MHz, CDCl₃) δ −79.11 (s, 3F).

### Step 2: (2R)-5,5,5-Trifluoro-4,4-dimethyl-2-[[(1R)-1-phenylethyl]amino]pentanenitrile and (2S)-5,5,5-trifluoro-4,4-dimethyl-2-[[(R)-1-phenylethyl]amino]pentanenitrile

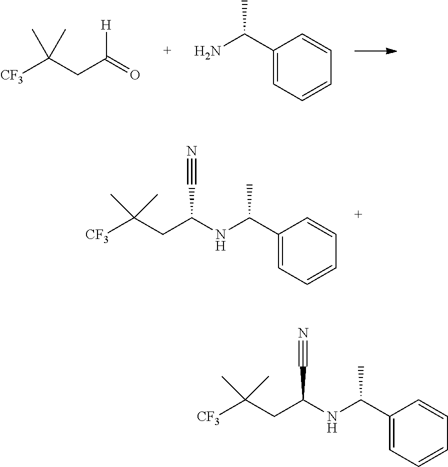

To a DCM (80 mL) solution of 4,4,4-trifluoro-3,3-dimethyl-butanal (113.7 g, 57.540 mmol) (purity about 7.8%) was added MeOH (110 mL). The mixture was cooled with ice-water bath. (1R)-1-phenylethanamine (8.46 g, 69.814 mmol) was added, followed by acetic acid (4.41 g, 73.436 mmol). The mixture was stirred at 0° C. for 10 min, then NaCN (3.56 g, 72.642 mmol) was added. The mixture was allowed to warm to rt slowly and stirred overnight. The reaction mixture was cooled to 0° C. and a solution of potassium carbonate (4 g) in water (20 mL) was added dropwise, followed by brine (40 mL). The mixture was extracted with DCM (2×100 mL). The organic layers were dried with sodium sulfate, filtered and concentrated. The residue was purified by flash chromatography (120 g silica gel, heptanes/EtOAc 0-30%) to afford a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 91%) as a colorless oil. ESI-MS m/z calc. 284.15002, found 285.2 (M+1)⁺; Retention time: 3.38 minutes; LC method F.

### Step 3: (2R)-5,5,5-Trifuoro-4,4-dimethyl-2-[[(1R)-1-phenylethyl]amino]pentanamide and (2S)-5,5,5-trifluoro-4,4-dimethyl-2-[[(1R)-1-phenylethyl]amino]pentanamide

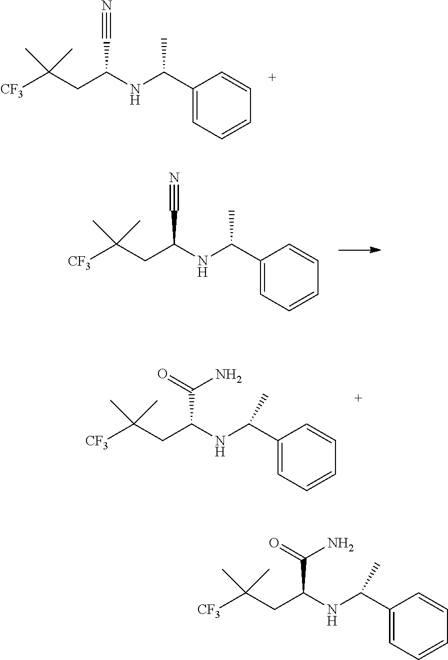 [SMILES: C[C@@H](N[C@@H](C#N)CC(C)(C)C(F)(F)F)c1ccccc1 ; C[C@@H](N[C@H](C#N)CC(C)(C)C(F)(F)F)c1ccccc1 ; C[C@@H](N[C@H](CC(C)(C)C(F)(F)F)C(N)=O)c1ccccc1 ; C[C@@H](N[C@@H](CC(C)(C)C(F)(F)F)C(N)=O)c1ccccc1]

To a solution of a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 52.300 mmol) in DCM (105 mL) was added sulfuric acid (56.3 g, 551.06 mmol). The mixture was stirred at rt overnight, poured on crude ice (200 g) and neutralized to pH 9 with 28% NH₃ in water (100 mL). The mixture was extracted with DCM (500 mL). The organic layer was dried with sodium sulfate, filtered and concentrated. The residue was purified by flash chromatography (330 g silica gel, heptanes/EtOAc 20-50%) to afford (2R)-5,5,5-trifluoro-4,4-dimethyl-2-[[(1R)-1-phenylethyl]amino]pentanamide (10.77 g, 68%) as a white solid. ¹H NMR (300 MHz, CDCl₃) δ 7.39-7.22 (m, 5H), 6.35 (br. s., 1H), 5.55 (br. s., 1H), 3.65 (q, J=6.5 Hz, 1H), 2.93 (dd, J=7.6, 3.8 Hz, 1H), 1.87 (dd, J=15.0, 3.8 Hz, 1H), 1.65-1.56 (m, 2H), 1.35 (d, J=6.5 Hz, 3H), 1.04 (s, 3H), 1.00 (s, 3H). ¹⁹F NMR (282 MHz, CDCl₃) δ −78.77 (s, 3F). 99.4% de by 19F NMR.

### Step 4: (2R)-5,5,5-Trifluoro-4,4-dimethyl-2-[[(1R)-1-phenylethyl]amino]pentanoic acid

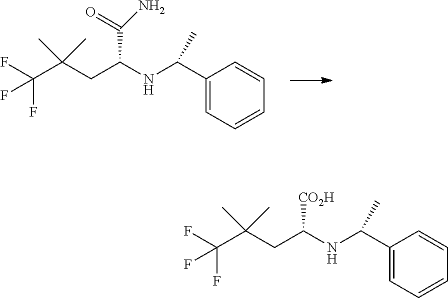 [SMILES: C[C@@H](N[C@H](CC(C)(C)C(F)(F)F)C(N)=O)c1ccccc1 ; C[C@@H](N[C@H](CC(C)(C)C(F)(F)F)C(=O)O)c1ccccc1]

To a solution of (2R)-5,5,5-trifluoro-4,4-dimethyl-2-[[(1R)-1-phenylethyl]amino]pentanamide (11.35 g, 37.541 mmol) in HOAc (50 mL) was added conc. HCl (65 mL of 11.8 M, 767.00 mmol), followed by water (50 mL). A white precipitate appeared. The mixture was heated at 100° C. for 66 h. More conc. HCl (40 mL of 11.8 M, 472.00 mmol) and HOAc (10 mL) were added. The mixture was stirred at 100° C. overnight. More HCl in water (20 mL of 6 M, 120.00 mmol) was added. After 7 h at 100° C., more HCl in water (20 mL of 6 M, 120.00 mmol) was added. The mixture was stirred at 100° C. overnight. It became a clear solution. More HCl in water (20 mL of 6 M, 120.00 mmol) was added. The mixture was stirred at 100° C. for 7 h, more HCl in water (20 mL of 6 M, 120.00 mmol) was added. The mixture was stirred at 100° C. overnight. The mixture was concentrated and co-evaporated with water (50 mL). The residue (17 g) was mixed with water (25 mL) at 50° C. for 20 min, cooled with ice-water bath for 20 min and filtered. The crude product was mixed with 1,4-dioxane (60 mL). The mixture was concentrated and dried on vacuum overnight to give (2R)-5,5,5-trifluoro-4,4-dimethyl-2-[[(1R)-1-phenylethyl]amino]pentanoic acid (hydrochloride salt) (13.04 g, 97%) as an off-white solid. ¹H NMR (300 MHz, DMSO-d6) δ 10.09 (br. s., 1H), 7.54-7.31 (m, 5H), 7.29-7.05 (m, 1H), 4.07 (q, J=5.9 Hz, 1H), 3.16-2.98 (m, 1H), 2.08-1.83 (m, 2H), 1.49 (d, J=6.5 Hz, 3H), 0.99 (s, 3H), 0.92 (s, 3H). ¹⁹F NMR (282 MHz, DMSO-d6) δ −78.28 (s, 3F). ESI-MS m/z calc. 303.14462, found 304.2 (M+1)⁺; Retention time: 1.98 minutes; LC method F.

### Step 5: (2R)-5,5,5-Trifluoro-4,4-dimethyl-2-[[(1R)-1-phenylethyl]amino]pentan-1-ol

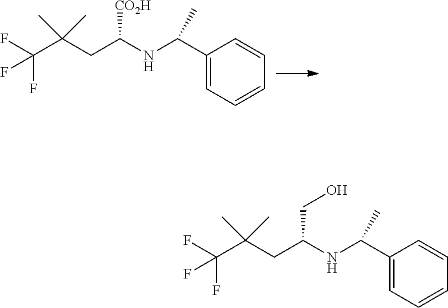 [SMILES: C[C@@H](N[C@H](CC(C)(C)C(F)(F)F)C(=O)O)c1ccccc1 ; C[C@@H](N[C@@H](CO)CC(C)(C)C(F)(F)F)c1ccccc1]

To a suspension of (2R)-5,5,5-trifluoro-4,4-dimethyl-2-[[(1R)-1-phenylethyl]amino]pentanoic acid (hydrochloride salt) (13.04 g, 36.267 mmol) in THE (200 mL) at 35° C. was added LAH in THE (100 mL of 1 M, 100.00 mmol) dropwise. The mixture was stirred at 40° C. for 2 h, cooled to 10° C. with ice-water bath and diluted with THE (200 mL). A mixture of water (3.8 g) and THE (50 mL) was added dropwise, followed by 25% aqueous NaOH (3.8 g) and water (10 g). The resulting mixture was stirred at rt for 30 min and at 50° C. for 1 h, filtered and washed with warm THF. The filtrate was concentrated to give 12.02 g of product (free amine) as a colorless oil. ¹H NMR (300 MHz, CDCl₃) δ 7.37-7.24 (m, 5H), 3.82 (q, J=6.5 Hz, 1H), 3.72-3.67 (m, 1H), 3.21 (dd, J=10.6, 4.7 Hz, 1H), 2.67 (quin, J=4.6 Hz, 1H), 1.66 (dd, J=14.7, 5.9 Hz, 1H), 1.54-1.45 (m, 1H), 1.36 (d, J=6.5 Hz, 3H), 1.03 (s, 3H), 0.97 (s, 3H). ¹⁹F NMR (282 MHz, CDCl₃) δ −78.83 (s, 3F). The above crude product (12.02 g) was dissolved in diethyl ether (20 mL) and diluted with heptanes (80 mL) and cooled in an ice-water bath. HCl in 1,4-dioxane (10.5 mL of 4 M, 42.000 mmol) was added dropwise. The mixture was stirred at rt for 30 min and filtered to give (2R)-5,5,5-trifluoro-4,4-dimethyl-2-[[(1R)-1-phenylethyl]amino]pentan-1-ol (hydrochloride salt) (11.56 g, 98%) as a white solid. ¹H NMR (300 MHz, DMSO-d6) δ 9.57 (br. s., 1H), 9.25 (t, J=9.8 Hz, 1H), 7.80-7.59 (m, 2H), 7.53-7.32 (m, 3H), 5.63 (br. s., 1H), 4.58 (t, J=6.3 Hz, 1H), 3.81-3.65 (m, 1H), 3.64-3.51 (m, 1H), 2.91-2.74 (m, 1H), 1.98-1.85 (m, 1H), 1.85-1.74 (m, 1H), 1.63 (d, J=6.8 Hz, 3H), 0.91 (s, 3H), 0.88 (s, 3H). ¹⁹F NMR (282 MHz, DMSO-d6) δ −77.71 (s, 3F). ESI-MS m/z calc. 289.16534, found 290.2 (M+1)⁺; Retention time: 2.08 minutes; LC method F.

### Step 6: (2R)-2-Amino-5,5,5-trifluoro-4,4-dimethyl-pentan-1-ol

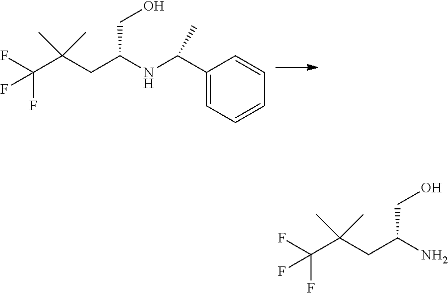 [SMILES: C[C@@H](N[C@@H](CO)CC(C)(C)C(F)(F)F)c1ccccc1 ; CC(C)(C[C@@H](N)CO)C(F)(F)F]

To a solution of (2R)-5,5,5-trifluoro-4,4-dimethyl-2-[[(1R)-1-phenylethyl]amino]pentan-1-ol (hydrochloride salt) (11.56 g, 35.482 mmol) in EtOH (200 mL) was added 10% palladium on carbon, 50% wet (5 g, 2.3492 mmol). The mixture was hydrogenated in a Parr shaker hydrogenation apparatus at 40 psi of hydrogen at rt for 9 h. More 10% palladium on carbon, 50% wet (1 g, 0.4698 mmol) was added. The mixture was shaken at 40 psi for 7 h. The mixture was filtered through Celite and washed with EtOH. The filtrate was concentrated. The residue (7.9 g) was triturated with a mixture of 2-methyltetrahydrofuran (28 mL) and heptanes (200 mL) and stirred overnight. The mixture was filtered, and the white solid was dried on vacuum to give (2R)-2-amino-5,5,5-trifluoro-4,4-dimethyl-pentan-1-ol (hydrochloride salt) (7.66 g, 93%) as a white solid. ¹H NMR (300 MHz, DMSO-d6) δ 8.08 (br. s., 3H), 5.46 (t, J=5.0 Hz, 1H), 3.67-3.52 (m, 1H), 3.43 (dt, J=11.7, 5.8 Hz, 1H), 3.29-3.16 (m, 1H), 1.88-1.73 (m, 1H), 1.72-1.58 (m, 1H), 1.15 (s, 3H), 1.10 (s, 3H). ¹⁹F NMR (282 MHz, DMSO-d6) δ −78.07 (s, 3F). ESI-MS m/z calc. 185.10275, found 186.2 (M+1)⁺; Retention time: 0.64 minutes; LC method F.

### Step 7: 3-[[4-[(2R)-2-Amino-5,5,5-trifluoro-4,4-dimethyl-pentoxy]-6-(2,6-dimethylphenyl)pyrimidin-2-yl]sulfamoyl]benzoic acid

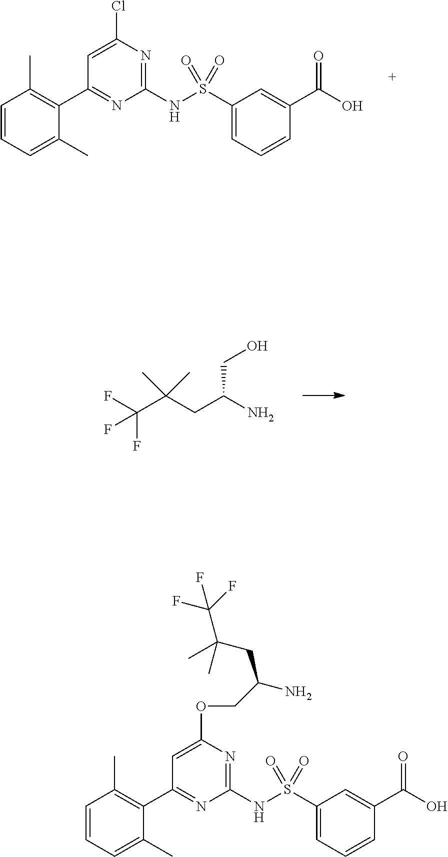 [SMILES: Cc1cccc(C)c1-c1cc(Cl)nc(NS(=O)(=O)c2cccc(C(=O)O)c2)n1 ; CC(C)(C[C@@H](N)CO)C(F)(F)F ; Cc1cccc(C)c1-c1cc(OC[C@H](N)CC(C)(C)C(F)(F)F)nc(NS(=O)(=O)c2cccc(C(=O)O)c2)n1]

3-[[4-Chloro-6-(2,6-dimethylphenyl)pyrimidin-2-yl]sulfamoyl]benzoic acid (6.12 g, 14.65 mmol) and (2R)-2-amino-5,5,5-trifluoro-4,4-dimethyl-pentan-1-ol (hydrochloride salt) (3.27 g, 14.75 mmol) were combined in THE (30 mL) and the resulting suspension was cooled in a water-ice bath. Sodium tert-butoxide (5.63 g, 58.58 mmol) was added inducing rapid partial dissolution of the solid. After 5 minutes, the cooling bath was removed, and the reaction was stirred at room temperature for 1 hour (90% conversion). More (2R)-2-amino-5,5,5-trifluoro-4,4-dimethyl-pentan-1-ol (hydrochloride salt) (363 mg, 1.638 mmol) was added and the mixture was stirred for one hour (no change). More sodium tert-butoxide (744 mg, 7.742 mmol) was added and the mixture was stirred for 40 min (96% conversion). Ethyl acetate (100 mL), HCl (90 mL of 1 M, 90.00 mmol) and brine (50 mL) were added, and the resulting two phases were separated. The organic phase was washed with brine (50 mL), dried over sodium sulfate and concentrated. The residue was triturated in EtOAc/MeOH/Hexanes and the solvents were evaporated to give 3-[[4-[(2R)-2-amino-5,5,5-trifluoro-4,4-dimethyl-pentoxy]-6-(2,6-dimethylphenyl)pyrimidin-2-yl]sulfamoyl]benzoic acid (hydrochloride salt) (8.88 g, 93%) as a cream solid. ¹H NMR (400 MHz, DMSO-d6) δ 13.15 (very broad s, 1H), 8.61-8.30 (m, 4H), 8.14 (dd, J 7.9, 1.9 Hz, 2H), 7.69 (t, J 7.8 Hz, 1H), 7.31-7.20 (m, 1H), 7.12 (d, J 7.6 Hz, 2H), 6.33 (s, 1H), 4.43 (dd, J 11.9, 3.3 Hz, 1H), 4.29-4.15 (m, 1H), 3.74 (s, 1H), 2.06-1.94 (broad m, 6H), 1.94-1.85 (m, 2H), 1.22 (s, 3H), 1.16 (s, 3H). ESI-MS m/z calc. 566.1811, found 567.62 (M+1)⁺; Retention time: 1.13 minutes (LC method A).

### Example 5: Preparation of 3-[[4-[(2R)-2-amino-3-[1-(trifluoromethyl)cyclopropyl]propoxy]-6-(2,6-dimethylphenyl)pyrimidin-2-yl]sulfamoyl]benzoic acid

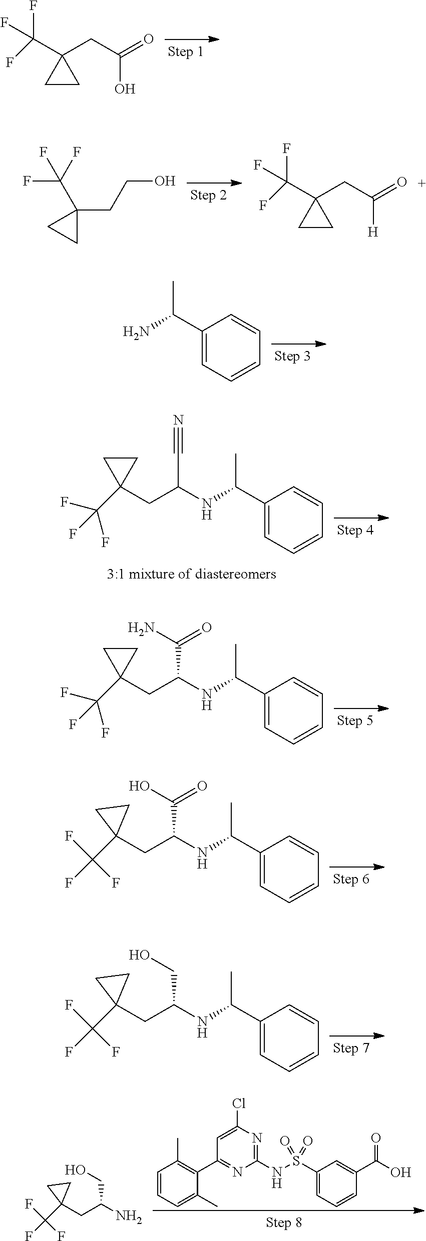

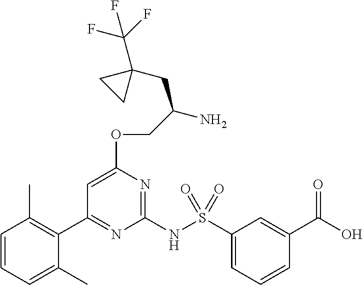 [SMILES: Cc1cccc(C)c1-c1cc(OC[C@H](N)CC2(C(F)(F)F)CC2)nc(NS(=O)(=O)c2cccc(C(=O)O)c2)n1]

### Step 1: 2-[1-(Trifluoromethyl)cyclopropyl]ethanol

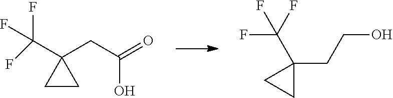 [SMILES: O=C(O)CC1(C(F)(F)F)CC1 ; OCCC1(C(F)(F)F)CC1]

LAH (49.868 g, 1.3139 mol) was added to THE (1700 mL) under nitrogen and the mixture was stirred for 30 minutes before being cooled to 0° C. 2-[1-(trifluoromethyl)cyclopropyl]acetic acid (190.91 g, 1.0107 mol) in THE (500 mL) was added dropwise while controlling the temperature <5° C. The mixture was allowed to warm up to room temperature and stirred for 24 hours. The resulting suspension was cooled to 0° C., water (50 mL) was added very slowly, followed by 15% w/w sodium hydroxide (50 mL) and water (150 mL). The mixture was stirred at 0° C. for 30 minutes, and filtered through Celite pad, the filter cake was washed with THE (2×500 mL). The combined filtrates were evaporated in vacuo to give 2-[1-(trifluoromethyl)cyclopropyl]ethanol (160.27 g, 98%) as amber oil containing ˜5% w/w of THE (by NMR). ¹H NMR (250 MHz, DMSO-d6) δ 4.57 (t, J 5.2 Hz, 1H), 3.55-3.39 (m, 2H), 1.74 (t, J 7.3 Hz, 2H), 1.00-0.58 (m, 4H).

### Step 2: 2-[1-(Trifluoromethyl)cyclopropyl]acetaldehyde

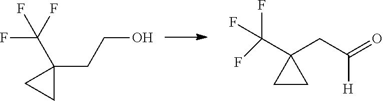 [SMILES: OCCC1(C(F)(F)F)CC1 ; O=CCC1(C(F)(F)F)CC1]

To a solution of 2-[1-(trifluoromethyl)cyclopropyl]ethanol (80 g, 467.1 mmol) in methylene chloride (1.1 L) was stirred at room temperature and treated with Dess-Martin periodinane (250 g, 589.4 mmol) portionwise (exothermic! cooled in ice bath and kept T<15° C.). To the mixture was added water (12 mL, 666.1 mmol) slowly added over 0.5 h (exothermic during addition up to 33° C., kept between 2° and 33° C. by cooling with cold water) giving a thick suspension. After the addition, the pale-yellow fine suspension was stirred at room temperature for 18 h. The yellow suspension was diluted with diethylether (500 mL) (yellow suspension) and stirred for 30 min. The slurry was filtered over Celite and the precipitate washed with 100 mL of diethylether. The organic phase was carefully treated with a saturated aqueous solution of sodium carbonate (500 ml, strong gas evolution, pH ˜10 at the end). The three-phase mixture was stirred at room temperature for 1 h and the solid was removed by filtration (large glass fritt). The phases (yellow cloudy Diethylether phase, colorless water phase) were separated and the organic phase was washed once more with a saturated aqueous solution of sodium carbonate (250 mL), once with 1M sodium thiosulfate (250 mL) and once with brine (250 mL). The aqueous phases were back extracted once with diethyl ether (150 mL) and the combined organic phases were dried, filtered and evaporated to give 2-[1-(trifluoromethyl)cyclopropyl]acetaldehyde (40 g, 56%) as a yellow liquid.

### Step 3: 2-[[(1R)-1-Phenylethyl]amino]-3-[1-(trifluoromethyl)cyclopropyl]propanenitrile

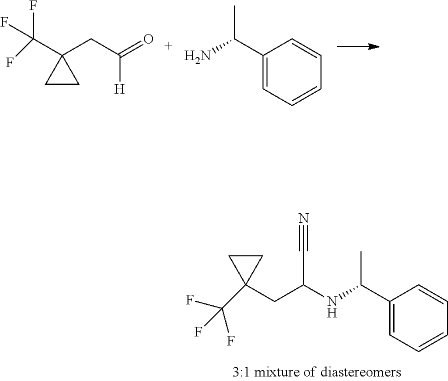

2-[1-(Trifluoromethyl)cyclopropyl]acetaldehyde (102 g, 670.5 mmol) in MeOH (700 mL) was treated with (1R)-1-phenylethanamine (86 mL, 667.1 mmol) and cooled in an ice bath. The solution was treated with acetic acid (38 mL, 668.2 mmol), stirred for 20 min in the ice bath, then solid NaCN (CAUTION, 33 g, 673.4 mmol) was added in one portion and the suspension was stirred in the melting ice bath for 14 hours. The solution was concentrated under reduced pressure and the residue was extracted with MTBE (1000 mL) and saturated sodium carbonate/water 1:1 (1000 mL) and washed with brine (350 mL). The aqueous phases were back extracted once with MTBE (250 mL) and the combined organic phases were dried, filtered and evaporated to give 2-[[(1R)-1-phenylethyl]amino]-3-[1-(trifluoromethyl)cyclopropyl]propanenitrile (180.8 g, 96%) as 3:1 mixture of diastereomers. ESI-MS m/z calc. 282.13437, found 283.0 (M+1)⁺; Retention time: 1.69 minutes (major isomer) and 1.62 minutes (minor isomer), LC method A.

### Step 4: (2R)-2-[[(1R)-1-Phenylethyl]amino]-3-[1-(trifluoromethyl)cyclopropyl]propenamide

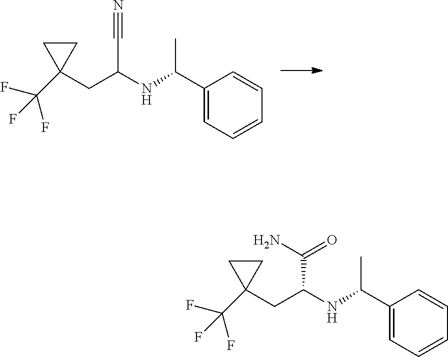 [SMILES: C[C@@H](NC(C#N)CC1(C(F)(F)F)CC1)c1ccccc1 ; C[C@@H](N[C@H](CC1(C(F)(F)F)CC1)C(N)=O)c1ccccc1]

In a 2 L flask equipped with mechanical stirring and a temperature probe, sulfuric acid (285 mL of 18 M, 5.130 mol) was added it was cooled in an ice bath. At an internal temperature of 5° C., a solution of 2-[[(1R)-1-phenylethyl]amino]-3-[1-(trifluoromethyl)cyclopropyl]propanenitrile (180.8 g, 640.4 mmol, 3:1 mixture of diastereomers) in DCM (900 mL) was added dropwise over 20 minutes. The ice bath was removed, and the deep orange emulsion was stirred at room temperature for 18 h and at 30-40° C. for 2 h. The deep orange emulsion was carefully added to a mixture of ice and water (2.2 L) under mechanical stirring to give a yellow three phase mixture which was basified by slow addition of ammonium hydroxide (1.33 L of 30% w/w, 10.25 mol) under ice cooling (very exothermic, internal temperature kept between 1° and 25° C. by adding ice). The yellow emulsion was stirred for 10 minutes at room temperature (pH ˜10), diluted with DCM (500 mL) and the phases were separated. The aqueous phase was washed twice more with DCM (400 and 200 mL) and the combined organic phases were washed once with water/brine 1:1 (500 mL). The DCM phase was dried, filtered and evaporated to give crude 2-[[(1R)-1-phenylethyl]amino]-3-[1-(trifluoromethyl)cyclopropyl]propanamide (189.5 g, 99%) as a yellow-orange oil. ESI-MS m/z calc. 300.14496, found 301.0 (M+1)⁺; Retention time: 1.40 minutes (major isomer) and 1.50 minutes (minor isomer) (3:1 mixture of diastereomers). The product was dissolved in ethanol (1.5 L) and it was treated quickly with HCl (240 mL of 4 M, 960.0 mmol) (4M in dioxane) and the resulting thick suspension was stirred at room temperature overnight under mechanic stirring. The solid was collected by filtration, washed with cold ethanol and dried under vacuum with a nitrogen bleed at 40-45° C. to give (2R)-2-[[(1R)-1-phenylethyl]amino]-3-[1-(trifluoromethyl)cyclopropyl]propanamide (hydrochloride salt) (147 g, 68%). ¹H NMR (499 MHz, DMSO-d6) δ 9.74 (d, J 67.9 Hz, 2H), 8.16-7.94 (m, 1H), 7.86 (s, 1H), 7.64-7.51 (m, 2H), 7.51-7.34 (m, 3H), 4.22 (s, 1H), 3.46-3.37 (m, 1H), 2.45 (d, J 15.9 Hz, 1H), 1.85 (dd, J 15.1, 10.4 Hz, 1H), 1.58 (d, J 6.7 Hz, 3H), 0.89 (pd, J 9.6, 9.2, 4.3 Hz, 2H), 0.84-0.66 (m, 2H). ESI-MS m/z calc. 300.14496, found 301.0 (M+1)⁺; Retention time: 1.40 minutes (major isomer) and 1.40 minutes (minor isomer), 97:3 mixture of diastereomers (LC method G).

### Step 5: (2R)-2-[[(1R)-1-Phenylethyl]amino]-3-[1-(trifluoromethyl)cyclopropyl]propanoic acid

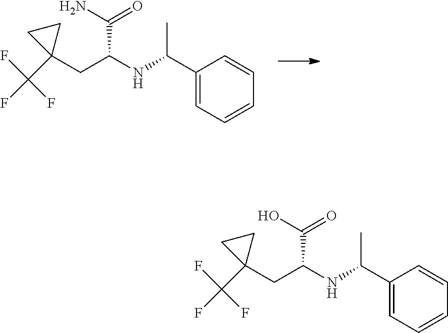 [SMILES: C[C@@H](N[C@H](CC1(C(F)(F)F)CC1)C(N)=O)c1ccccc1 ; C[C@@H](N[C@H](CC1(C(F)(F)F)CC1)C(=O)O)c1ccccc1]

In a 5 L flask equipped with mechanical stirring, (2R)-2-[[(1R)-1-phenylethyl]amino]-3-[1-(trifluoromethyl)cyclopropyl]propanamide (hydrochloride salt) (147 g, 436.5 mmol) was added to acetic acid (735 mL) under stirring and the thick colorless suspension was treated with HCl (1.3 L of 12 M, 15.60 mol). The colorless suspension was carefully heated to 60-65° C. (strong foaming, acetic acid (145 mL) was added) and the suspension was stirred at 60-65° C. for 16 h. The suspension was then slowly heated to 100° C. (over 4 h, strong foaming) and the resulting solution was stirred at 100° C. for another 20 h. The pale-yellow solution was concentrated under reduced pressure at 65° C. to a semisolid mass and it was treated with water (1.5 L). The thick suspension was heated to 70-80° C. and left to cool to room temperature under stirring for 2 h. The solid was collected by filtration, washed with water and sucked dry overnight. The wet solid was further dried under reduced pressure at 50-60° C. for 4 h to give (2R)-2-[[(1R)-1-phenylethyl]amino]-3-[1-(trifluoromethyl)cyclopropyl]propanoic acid (hydrochloride salt) (135 g, 92%) as an off-white solid. ESI-MS m/z calc. 301.12897, found 302.0 (M+1)⁺; Retention time: 1.82 minutes; (LC method G).

### Step 6: (2R)-2-[[(1R)-1-phenylethyl]amino]-3-[1-(trifluoromethyl)cyclopropyl]propan-1-ol

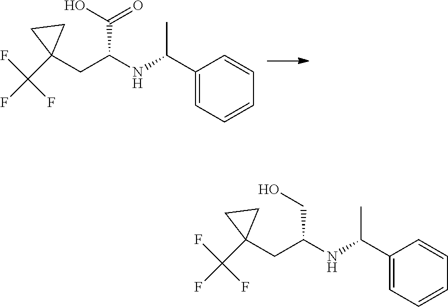 [SMILES: C[C@@H](N[C@H](CC1(C(F)(F)F)CC1)C(=O)O)c1ccccc1 ; C[C@@H](N[C@@H](CO)CC1(C(F)(F)F)CC1)c1ccccc1]

In a 5 L flask equipped with mechanical stirring and under dry nitrogen atmosphere, (2R)-2-[[(1R)-1-phenylethyl]amino]-3-[1-(trifluoromethyl)cyclopropyl]propanoic acid (hydrochloride salt) (135 g, 399.7 mmol) was suspended in THE (2 L) (thick suspension). It was heated to 35-40° C. and LAH (47.3 g, 1.214 mol) (pellets) was slowly added over 1 hour, while keeping the internal temperature between 3° and 40° C. by external cooling. The mixture was stirred for 1 hour at 30-40° C. (almost no hydrogen evolution anymore, grey suspension, most starting material in solution) and it was heated at 50-55° C. for 1 h. The grey suspension was left stirring in the cooling heating mantel overnight. The grey suspension was cooled in an ice bath and quenched by careful addition of water (44 mL, 2.442 mol), NaOH (41 mL of 6 M, 246.0 mmol) and water (44 mL, 2.442 mol) (high exotherm with first water addition, kept between 5° C. and 30° C. by cooling). The grey suspension was heated to 50-55° C. for 1 h, by which time a colorless suspension was obtained. The warm suspension was filtered over a pad of Celite covered over magnesium sulfate. The solids were washed with hot THF and evaporated to give crude (2R)-2-[[(1R)-1-phenylethyl]amino]-3-[1-(trifluoromethyl)cyclopropyl]propan-1-ol (121 g, 105%) as an oil. The crude was dissolved in diethyl ether (1 μL, clear solution) and slowly treated with HCl (101 mL of 4 M, 404.0 mmol) (4M in dioxane) under cooling. The resulting thick suspension was stirred at room temperature for 1 h, the solid collected by filtration, washed with diethyl ether and dried under reduced pressure at 40-45° C. with a nitrogen bleed to give (2R)-2-[[(1R)-1-phenylethyl]amino]-3-[1-(trifluoromethyl)cyclopropyl]propan-1-ol (hydrochloride salt) (126.6 g, 98%) as an off-white solid. ¹H NMR (500 MHz, DMSO-d6) δ 9.34 (s, 2H), 7.66 (d, J 7.4 Hz, 2H), 7.43 (dt, J 25.1, 7.4 Hz, 3H), 5.59 (s, 1H), 4.58 (q, J 6.6 Hz, 1H), 3.83 (d, J=12.6 Hz, 1H), 3.62-3.54 (m, 1H), 2.89 (s, 1H), 2.33-2.24 (m, 1H), 1.67-1.51 (m, 4H), 0.97-0.81 (m, 3H), 0.71 (s, 1H). ESI-MS m/z calc. 287.1497, found 288.0 (M+1)⁺; Retention time: 0.99 minutes (LC method A).

### Step 7: (2R)-2-Amino-3-[1-(trifluoromethyl)cyclopropyl]propan-1-ol

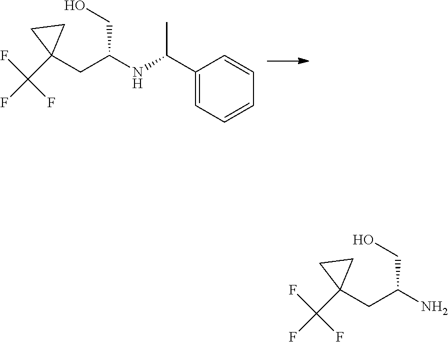 [SMILES: C[C@@H](N[C@@H](CO)CC1(C(F)(F)F)CC1)c1ccccc1 ; N[C@@H](CO)CC1(C(F)(F)F)CC1]

In a 1 L hydrogenation reactor, (2R)-2-[[(1R)-1-phenylethyl]amino]-3-[1-(trifluoromethyl)cyclopropyl]propan-1-ol (hydrochloride salt) (63.3 g, 195.5 mmol) was dissolved in EtOH (630 mL) (under warming), and it was treated with Pd/C (6.3 g of 10% w/w, 5.920 mmol) (12.5 g of 50% water wet) and the reaction was stirred under 2 bar of hydrogen at 40° C. for 24 h. The reaction mixture was filtered over Celite. The pad was washed with ethanol and the colorless filtrate was evaporated to a solid mass, which was triturated with diethyl ether. The suspension was stirred at room temperature for 1 h. The solid was filtered, washed with plenty of diethyl ether and dried to give (2R)-2-amino-3-[1-(trifluoromethyl)cyclopropyl]propan-1-ol (hydrochloride salt) (41.8 g, 97%) as an off-white solid. ¹H NMR (500 MHz, DMSO-d6) δ 8.18 (s, 3H), 5.45 (t, J 4.9 Hz, 1H), 3.71 (dt, J 11.6, 3.9 Hz, 1H), 3.55 (dt, J 11.2, 5.4 Hz, 1H), 3.24 (h, J 4.7 Hz, 1H), 2.08 (dd, J 15.1, 5.4 Hz, 1H), 1.69 (dd, J 15.1, 9.4 Hz, 1H), 0.97 (h, J 6.5, 5.9 Hz, 2H), 0.86 (s, 2H). ESI-MS m/z calc. 183.0871, found 184.0 (M+1)⁺; Retention time: 0.65 minutes; LC method A.

### Step 8: 3-[[4-[(2R)-2-Amino-3-[1-(trifluoromethyl)cyclopropyl]propoxy]-6-(2,6-dimethylphenyl)pyrimidin-2-yl]sulfamoyl]benzoic acid

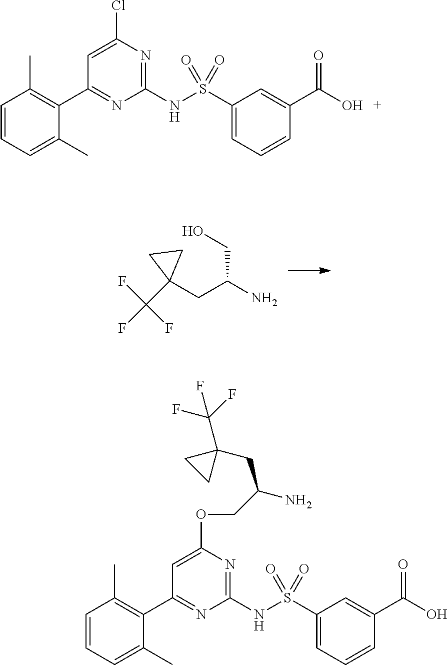

3-[[4-Chloro-6-(2,6-dimethylphenyl)pyrimidin-2-yl]sulfamoyl]benzoic acid (19.09 g, 45.68 mmol) and (2R)-2-amino-3-[1-(trifluoromethyl)cyclopropyl]propan-1-ol (hydrochloride salt) (10.18 g, 46.35 mmol) were dissolved in THE (100 mL) and cooled in an ice water bath. Sodium tert-butoxide (18.14 g, 188.8 mmol) was added and the reaction was allowed to warm to room temperature. The reaction was stirred for 1 h, then partitioned between ethyl acetate (500 mL) and aqueous HCl (275 mL of 1 M, 275.0 mmol). The organics were separated, washed with brine, dried over sodium sulfate and evaporated to give 3-[[4-[(2R)-2-amino-3-[1-(trifluoromethyl)cyclopropyl]propoxy]-6-(2,6-dimethylphenyl)pyrimidin-2-yl]sulfamoyl]benzoic acid (hydrochloride salt) (26.74 g, 94%). ESI-MS m/z calc. 564.1654, found 565.1 (M+1)⁺; Retention time: 0.48 minutes; LC method B.

### Example 6: Preparation of 3-[[4-[(2R)-2-amino-3-cyclopropyl-propoxy]-6-(2,6-dimethylphenyl)pyrimidin-2-yl]sulfamoyl]benzoic acid

### Step 1: tert-Butyl N-[(1R)-1-(cyclopropylmethyl)-2-hydroxy-ethyl]carbamate

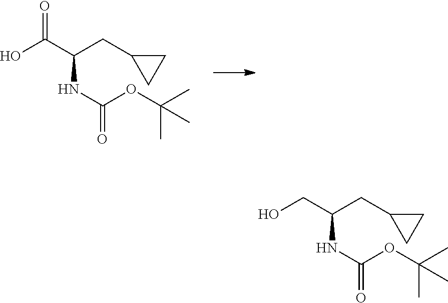 [SMILES: CC(C)(C)OC(=O)N[C@H](CC1CC1)C(=O)O ; CC(C)(C)OC(=O)N[C@@H](CO)CC1CC1]

A solution of (2R)-2-(tert-butoxycarbonylamino)-3-cyclopropyl-propanoic acid (0.22 g, 0.9596 mmol) and Borane-Tetrahydrofuran Complex (2.9 mL of 1 M, 2.900 mmol) in THE (5 mL) was stirred for three hours. The reaction was quenched with 1 M citric acid and extracted with ethyl acetate. The combined extracts were washed with water, dried over sodium sulfate, and evaporated under vacuum to give tert-butyl N-[(1R)-1-(cyclopropylmethyl)-2-hydroxy-ethyl]carbamate (89 mg, 43%). ESI-MS m/z calc. 215.15215, found 216.2 (M+1)⁺; Retention time: 0.47 minutes; LC method B.

### Step 2: 3-[[4-[(2R)-2-(tert-Butoxycarbonylamino)-3-cyclopropyl-propoxy]-6-(2,6-dimethylphenyl)pyrimidin-2-yl]sulfamoyl]benzoic acid

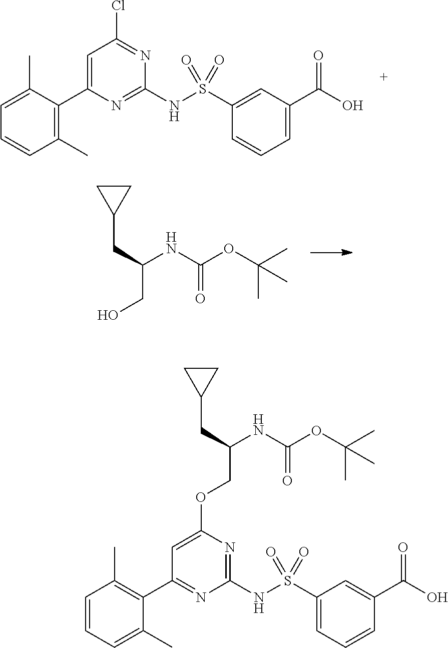

A solution of 3-[[4-chloro-6-(2,6-dimethylphenyl)pyrimidin-2-yl]sulfamoyl]benzoic acid (approximately 172.7 mg, 0.4134 mmol), tert-Butyl N-[(1R)-1-(cyclopropylmethyl)-2-hydroxy-ethyl]carbamate (89 mg, 0.4134 mmol), and sodium t-butoxide (approximately 159.0 mg, 1.654 mmol) in THE (2.067 mL) was stirred for 22 hours. The reaction was quenched with 1 M citric acid, diluted with water, and extracted with ethyl acetate. The combined extracts were washed with brine, dried over sodium sulfate, and evaporated. The residue was purified by silica gel column chromatography with 0-10% methanol in dichloromethane to give partially clean product. The impure product was re-purified using a reverse phase HPLC-MS method using a Luna C₁₈(2) column (75×30 mm, 5 μm particle size) sold by Phenomenex (pn: 00C-4252-U0-AX), and a dual gradient run from 1-99% mobile phase B over 15.0 minutes. Mobile phase A=H₂O (5 mM HCl). Mobile phase B=CH₃CN. Flow rate=50 mL/min, and column temperature=25° C. to give 3-[[4-[(2R)-2-(tert-butoxycarbonylamino)-3-cyclopropyl-propoxy]-6-(2,6-dimethylphenyl)pyrimidin-2-yl]sulfamoyl]benzoic acid (45 mg, 18%) obtained as a colorless solid. ESI-MS m/z calc. 596.23047, found 597.3 (M+1)⁺; Retention time: 0.68 minutes; LC method B.

### Step 3: 3-[[4-[(2R)-2-Amino-3-cyclopropyl-propoxy]-6-(2,6-dimethylphenyl)pyrimidin-2-yl]sulfamoyl]benzoic acid

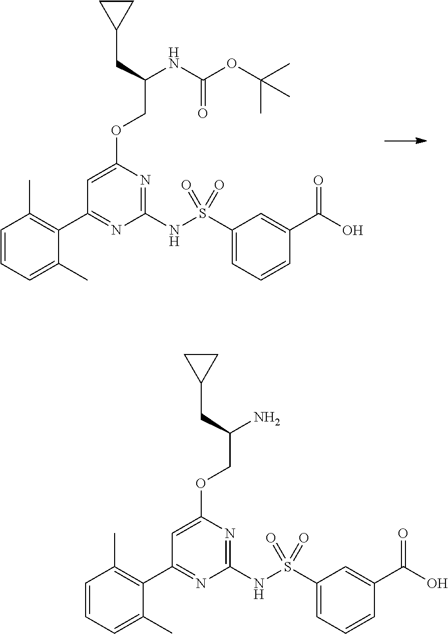 [SMILES: Cc1cccc(C)c1-c1cc(OC[C@@H](CC2CC2)NC(=O)OC(C)(C)C)nc(NS(=O)(=O)c2cccc(C(=O)O)c2)n1 ; Cc1cccc(C)c1-c1cc(OC[C@H](N)CC2CC2)nc(NS(=O)(=O)c2cccc(C(=O)O)c2)n1]

A solution of 3-[[4-[(2R)-2-(tert-butoxycarbonylamino)-3-cyclopropyl-propoxy]-6-(2,6-dimethylphenyl)pyrimidin-2-yl]sulfamoyl]benzoic acid (45 mg, 0.07542 mmol) in HCl (3 mL of 4 M, 12.00 mmol) (in dioxane) was stirred for four hours. The solvent was removed under vacuum, and the resulting solids were triturated with diethyl ether and dried under vacuum to give 3-[[4-[(2R)-2-amino-3-cyclopropyl-propoxy]-6-(2,6-dimethylphenyl)pyrimidin-2-yl]sulfamoyl]benzoic acid (hydrochloride salt) (53 mg, 132%). ESI-MS m/z calc. 496.17804, found 497.3 (M+1)⁺; Retention time: 0.41 minutes; LC method B.

### Example 7: Preparation of (2R)-2-amino-5,5-dimethyl-hexan-1-ol

### Step 1: Methyl 2-(tert-butoxycarbonylamino)-5,5-dimethyl-hex-2-enoate

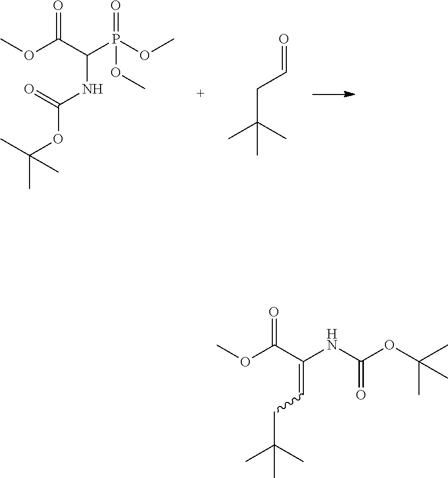

To a stirred solution of methyl 2-(tert-butoxycarbonylamino)-2-dimethoxyphosphoryl-acetate (16.4 g, 55.174 mmol) and DBU (8.0422 g, 7.9 mL, 52.827 mmol) in DCM (100 mL) was added at 0° C. (ice bath) 3,3-dimethylbutyraldehyde (5.0274 g, 6.3 mL, 50.194 mmol). The reaction mixture was stirred at room temperature for 16 h. Aqueous HCl (1 N) (100 mL) was added, and the phases were separated. The aqueous layer was washed with DCM (2×100 mL). The combined organic layers were dried with sodium sulfate, filtered, and concentrated under reduced pressure. The crude residue was purified by 50 g silica gel pad using a gradient of 15% EtOAc in heptanes to afford methyl 2-(tert-butoxycarbonylamino)-5,5-dimethyl-hex-2-enoate (13.6 g, 95%) as a clear oil that crystallized to a white solid. ¹H NMR (400 MHz, CDCl₃) δ 6.66 (t, J 7.6 Hz, 1H), 5.86 (br. s, 1H), 3.79 (s, 3H), 2.12 (d, J 7.6 Hz, 2H), 1.47 (s, 9H), 0.95 (s, 9H).

### Step 2: Methyl (2R)-2-(tert-butoxycarbonylamino)-5,5-dimethyl-hexanoate

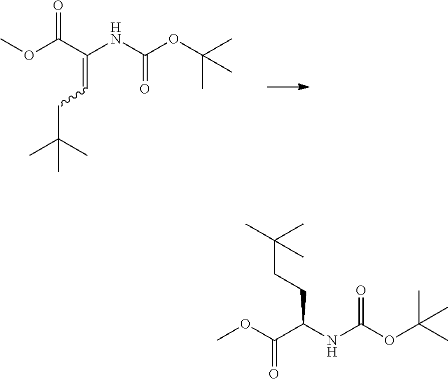 [SMILES: COC(=O)C(=CCC(C)(C)C)NC(=O)OC(C)(C)C ; COC(=O)[C@@H](CCC(C)(C)C)NC(=O)OC(C)(C)C]

To a solution of methyl 2-(tert-butoxycarbonylamino)-5,5-dimethyl-hex-2-enoate (13.630 g, 50.230 mmol) in ethanol (184 mL) and 1,4-dioxane (61 mL) was bubbled nitrogen for 5 min. Then, 1,2-bis[(2R,5R)-2,5-diethylphospholano]benzene(1,5-cyclooctadiene)rhodium(I) trifluoromethanesulfonate (363 mg, 0.5023 mmol) was added and the mixture was put in an ultrasound bath for 5 min under nitrogen. The reaction mixture was then purged with nitrogen gas (3×30 psi) then purged with hydrogen (3×50 psi). A pressure of 50 psi (3.5 bar) of hydrogen pressure was maintained and the reaction was stirred at room temperature for 16 h, at which time the volatiles were removed under reduced pressure and the residue was passed through a plug of silica gel (80 g) using an eluent of 15% EtOAc in heptanes to afford methyl (2R)-2-(tert-butoxycarbonylamino)-5,5-dimethyl-hexanoate (14 g, 97%) as a colorless oil. ¹H NMR (400 MHz, CDCl₃) δ 5.00 (d, J 7.3 Hz, 1H), 4.27 (d, J 5.4 Hz, 1H), 3.74 (s, 3H), 1.87-1.71 (m, 1H), 1.64-1.54 (m, 1H), 1.45 (s, 9H), 1.28-1.15 (m, 2H), 0.87 (s, 9H). ESI-MS m/z calc. 273.194, found 296.2 (M+23)⁺; Retention time: 4.58 minutes; LC method J.

### Step 3: tert-Butyl N-[(1R)-1-(hydroxymethyl)-4,4-dimethyl-pentyl]carbamate

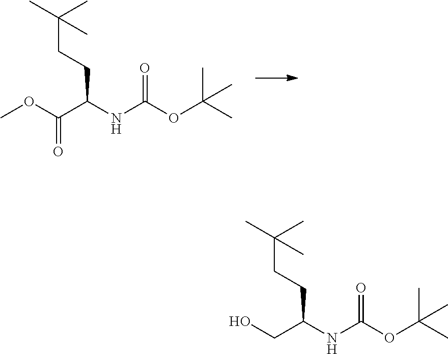 [SMILES: COC(=O)[C@@H](CCC(C)(C)C)NC(=O)OC(C)(C)C ; CC(C)(C)CC[C@H](CO)NC(=O)OC(C)(C)C]

To a solution of methyl (2R)-2-(tert-butoxycarbonylamino)-5,5-dimethyl-hexanoate (14 g, 48.652 mmol) in THE (145 mL) was added LiBH₄ (2 M solution in THF) (61 mL of 2 M, 122.00 mmol) (no exotherm observed). The reaction mixture was stirred at room temperature for 2.5 h. The reaction mixture was then poured slowly over a saturated aqueous solution of NH₄Cl (50 mL) at 0° C. (strong evolution of gas, but no exotherm). The product was extracted with EtOAc (3×150 mL). The combined organic layers were washed with brine (150 mL), dried with sodium sulfate, filtered and concentrated under reduced pressure to afford crude product tert-butyl N-[(1R)-1-(hydroxymethyl)-4,4-dimethyl-pentyl]carbamate (13.23 g, 89%) as a clear oil. ESI-MS m/z calc. 245.1991, found 268.2 (M+23)⁺; Retention time: 1.8 minutes. ¹H NMR (400 MHz, CDCl₃) δ 4.62 (br. s, 1H), 3.68 (d, J 7.1 Hz, 1H), 3.55 (d, J 8.1 Hz, 2H), 2.57 (br. s, 1H), 1.55-1.29 (m, 11H), 1.28-1.19 (m, 2H), 0.88 (s, 9H); LC method I.

### Step 4: (2R)-2-Amino-5,5-dimethyl-hexan-1-ol

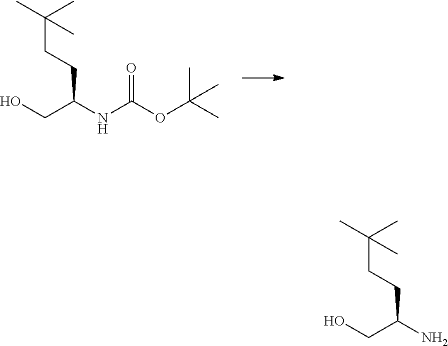

To a solution of tert-butyl N-[(1R)-1-(hydroxymethyl)-4,4-dimethyl-pentyl]carbamate (13.23 g, 43.460 mmol) in 1,4-dioxane (140 mL) was added hydrogen chloride (4 N in 1,4-dioxane) (63 mL of 4 M, 252.00 mmol). The reaction mixture was stirred at room temperature for 16 h then the mixture was evaporated to dryness under reduced pressure. The residue was triturated in THE then filtered to give (2R)-2-amino-5,5-dimethyl-hexan-1-ol (hydrochloride salt) (8.137 g, 98%) as a white solid. ¹H NMR (400 MHz, DMSO-d6) δ 7.97 (br. s, 3H), 5.28 (br. s, 1H), 3.58 (dd, J 11.4, 3.5 Hz, 1H), 3.44 (dd, J 11.4, 6.2 Hz, 1H), 3.02-2.89 (m, 1H), 1.58-1.43 (m, 2H), 1.30-1.13 (m, 2H), 0.86 (s, 9H). ESI-MS m/z calc. ESI-MS m/z calc. 145.14667, found 146.3 (M+1)⁺; Retention time: 1.78 minutes; LC method J.

### Example 8: Preparation of 3-[[4-[(2R)-2-amino-5,5-dimethyl-hexoxy]-6-(2,6-dimethylphenyl)pyrimidin-2-yl]sulfamoyl]benzoic acid

### Step 1: 3-[[4-[(2R)-2-Amino-5,5-dimethyl-hexoxy]-6-(2,6-dimethylphenyl)pyrimidin-2-yl]sulfamoyl]benzoic acid

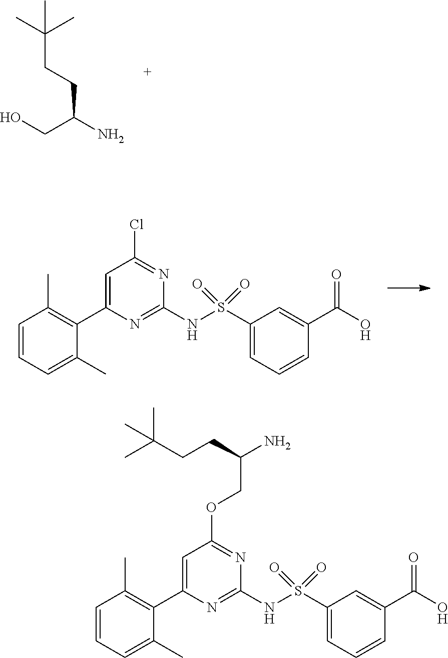

A solution of (2R)-2-amino-5,5-dimethyl-hexan-1-ol (hydrochloride salt) (4.495 g, 23.501 mmol) in anhydrous DMF (23 mL) was added to a solution of 3-[[4-chloro-6-(2,6-dimethylphenyl)pyrimidin-2-yl]sulfamoyl]benzoic acid (13.5 g, 32.384 mmol) in Me-THF (117 mL). The mixture was cooled down to 10-15° C. (inner temperature) and then sodium tert-butoxide (17.4 g, 181.05 mmol) was added. The reaction was stirred at 10-15° C. for 2 hours, then cooled down to 0° C. and quenched by the addition of an aqueous solution of 1 N HCl (180 mL) at 0° C. The biphasic mixture was stirred for 30 minutes. The layers were then separated, and the aqueous layer was extracted with 2-methyltetrahydrofuran (5×500 mL). The combined organic layers were washed with water (3×500 mL) and brine (1×500 mL), dried over magnesium sulfate, filtered and concentrated in vacuo. The residue was dissolved in MeOH (75 mL) and precipitated in EtOAc (800 mL). the solid was filtered over a glass frit (por.4) and the residue was collected using MeOH to dissolve it. The crude mixture was purified by reverse phase chromatography on a 275 g C₁₈ cartridge, eluting with a gradient of 0 to 80% of CH₃CN in acidic water (0.1% of hydrochloric acid in water) to afford after evaporation 3-[[4-[(2R)-2-amino-5,5-dimethyl-hexoxy]-6-(2,6-dimethylphenyl)pyrimidin-2-yl]sulfamoyl]benzoic acid (hydrochloride salt) (10.2 g, 70%) as a white solid. ¹H NMR (400 MHz, DMSO-d6) δ 8.45 (t, J 1.7 Hz, 1H), 8.38 (br. s, 3H), 8.18-8.09 (m, 2H), 7.70 (t, J 7.8 Hz, 1H), 7.29-7.21 (m, 1H), 7.12 (d, J 7.8 Hz, 2H), 6.31 (s, 1H), 4.43-4.36 (m, 1H), 4.35-4.27 (m, 1H), 3.45 (br. s, 1H), 1.99 (s, 6H), 1.70-1.49 (m, 2H), 1.31 (td, J 12.6, 4.9 Hz, 1H), 1.21-1.11 (m, 1H), 0.83 (s, 9H), 1H labile missing (from —COOH). ESI-MS m/z calc. 526.225, found 527.2 (M+1)⁺; Retention time: 2.58 minutes; LC method A.

### Example 9: Preparation of (2R)-2-amino-3-(1-methylcyclopropyl)propan-1-ol

### Step 1: 2-(1-Methylcyclopropyl)ethanol

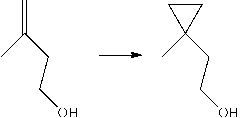

To a solution of diethylzinc (hexane solution) (2 L of 1 M, 2.0000 mol) was added 3-methylbut-3-en-1-ol (135 g, 1.5674 mol) at 0-15° C. in 30 minutes. The mixture was then warmed to 15° C. and after 20 minutes of stirring, diiodomethane (482.7 g, 1.8022 mol) as a DCM (270 mL) solution was added over 1 h. The reaction was then warmed to 25° C. and stirred for 20 hours. After cooling to 5° C., the reaction mixture was quenched with aqueous HCl (2 M, 1.35 L). The phases were split, and the aqueous phase extracted with DCM (2×675 mL). The hexanes extract was, washed with sodium thiosulfate (10% w/w, 1.35 L), and concentrated in vacuo. The sodium thiosulfate solution was then extracted with the two DCM extracts successively. The DCM extracts were then combined with the product obtained from the concentration of the hexanes extract. Water (1.35 L) was then added to this combined organic phase. The mixture was cooled to 2° C. and an aqueous solution of sodium permanganate (83.5 g, 40% w/w, 235.34 mmol) was added. The mixture was stirred at 2° C. for 15 minutes, sodium bisulfite (10% w/w, 1 L) was added, the phases were split and the aqueous phase washed with DCM (2×350 mL). The organic extracts were combined, dried over sodium sulfate, filtered and concentrated in vacuo and distilled (40° C., 2-5 mbar) to give 2-(1-methylcyclopropyl)ethanol (106.6 g, 66%) as a clear oil. ¹H NMR (400 MHz, CDCl₃) δ 3.75 (t, J 7.0 Hz, 2H), 1.51 (t, J 7.0 Hz, 3H), 1.04 (s, 3H), 0.32-0.22 (m, 4H).

### Step 2: 2-(1-Methylcyclopropyl)acetaldehyde

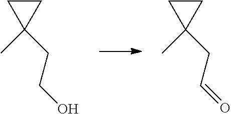

2-(1-Methylcyclopropyl)ethanol (106 g, 1.0319 mol) was added to a mixture of water (800 mL) and DCM (800 mL) before sodium bromide (10.6 g, 103.02 mmol), sodium bicarbonate (200 g, 2.3808 mol) and TEMPO (1.6 g, 10.240 mmol) were successively added. The mixture was cooled down to 0° C. and a NaOCl aqueous solution (1.3 L of 0.8 M, 1.0400 mol) was added over 1 hour (T=1.0 to 8.2° C.). After 1 hour, the mixture was filtered over celite (0.5 part) and the phases were separated. The aqueous phase was extracted with DCM (2×3.5 vol). The combined organic phases were dried with sodium sulfate (0.5 part) and concentrated under reduced pressure (300 mbar, bath: 30° C.) to give a slightly amber DCM solution with a content of 4.15% w/w of 2-(1-methylcyclopropyl)acetaldehyde (101.27 g, 100%). ¹H NMR (400 MHz, Chloroform-d) δ 9.85-9.79 (m, 1H), 2.26 (d, J 2.4 Hz, 2H), 1.13 (s, 3H), 0.45 (br d, J=6.4 Hz, 4H).

### Step 3: 3-(1-Methylcyclopropyl)-2-[[(1R)-1-phenylethyl]amino]propanenitrile

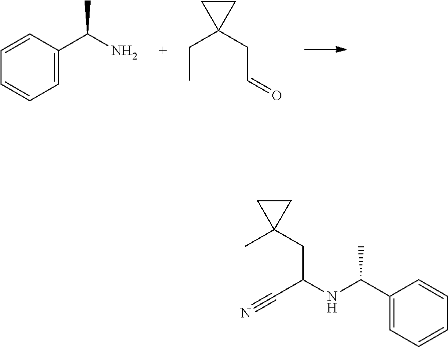

To a solution of 2-(1-methylcyclopropyl)acetaldehyde (101.27 g, 1.0319 mol) in MeOH (850 mL), cooled to 0° C. in an ice bath, was added portionwise (1R)-1-phenylethanamine (122.20 g, 130 mL, 1.0084 mol) (T increased from 3° C. to 9° C.). Acetic acid (68.640 g, 65 mL, 1.1430 mol) was added dropwise (T increased from 3° C. to 5° C.) followed by the portionwise addition of sodium cyanide (53 g, 1.0815 mol) (T increased from 1° C. to 5° C.). The mixture was allowed to warm to room temperature and stirred overnight. The mixture was concentrated under vacuum (rotovapor connected to a scrubber containing a 6 M aqueous solution of sodium hydroxide). To the residue, MTBE (5 vol.) and an aqueous solution of potassium carbonate (10% w/w, 5 vol) were added. The mixture was stirred 5 min before the phases were split. The organic layer was washed with brine (15% w/w, 3×5 vol.), dried with sodium sulfate (0.5 part) and concentrated under reduced pressure to afford 3-(1-methylcyclopropyl)-2-[[(1R)-1-phenylethyl]amino]propanenitrile (diastereomeric mixture 70:30, 245.86 g, 92%) as a slightly amber oil. ESI-MS m/z calc. 228.16264, found 229.2 (M+1)⁺; Retention time: 2.935 minutes; LC method F.

### Step 4: (2R)-3-(1-Methylcyclopropyl)-2-[[(1R)-1-phenylethyl]amino]propanamide

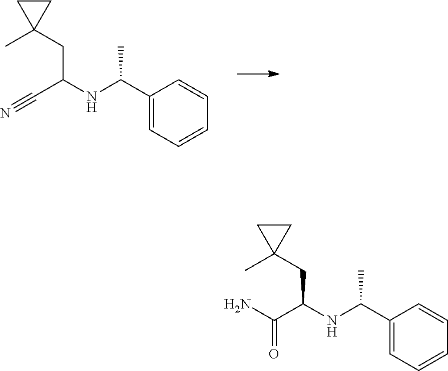 [SMILES: C[C@@H](NC(C#N)CC1(C)CC1)c1ccccc1 ; C[C@@H](N[C@H](CC1(C)CC1)C(N)=O)c1ccccc1]

To a solution combining two batches of 3-(1-methylcyclopropyl)-2-[[(1R)-1-phenylethyl]amino]propanenitrile (diastereomeric mixture 70:30, 245 g, 948.53 mmol, and 19 g, 68.899 mmol) in a mixture of DMSO (1.2 L) and water (250 mL) stirred at 50° C., was added potassium carbonate (33 g, 238.77 mmol). An aqueous solution of hydrogen peroxide (220 mL of 9.8 M, 2.1560 mol) was added dropwise over 1.5 hours. The mixture was stirred for 1 hour at 50° C. The mixture was cooled down to room temperature before water (5 μL, 20 vol) was added. The aqueous layer was extracted with MTBE (2×1.5 μL, 2×6 vol). The combined organic layers were isolated and extracted with an aqueous solution of HCl (2×1.5 L of 1 M, 2×6 vol). The acidic aqueous layers were combined and slowly stirred at 5° C. for 2 hours. The resulting suspension was filtered and the solids recovered were dried under reduced pressure at 50° C. for 2 hours to afford (2R)-3-(1-methylcyclopropyl)-2-[[(1R)-1-phenylethyl]amino]propanamide (hydrochloride salt) (151.24 g, 52%) as a white powder. ESI-MS m/z calc. 246.1732, found 274.2 (M+1)⁺; Retention time: 1.391 minutes. ¹H NMR (400 MHz, DMSO-d6) δ 10.04-9.89 (m, 1H), 9.56-9.37 (m, 1H), 8.05 (s, 1H), 7.77-7.56 (m, 3H), 7.47-7.37 (m, 3H), 4.28-4.14 (m, 1H), 3.45-3.28 (m, 1H), 2.20-2.07 (m, 1H), 1.59 (d, J 6.8 Hz, 3H), 1.44 (dd, J 13.4, 10.8 Hz, 1H), 0.92 (s, 3H), 0.35-0.11 (m, 4H); LC method F.

To the mother liquor was added sodium chloride (500 g, 2 parts) and the aqueous solution was stirred at 5° C. overnight. The resulting suspension was filtered and the solids recovered were dried with an air flow for 2 hours to afford 3-(1-methylcyclopropyl)-2-[[(1R)-1-phenylethyl]amino]propanamide (hydrochloride salt) (89.42 g, 31%) as a white powder isomeric mixture. ESI-MS m/z calc. 246.1732, found 247.2 (M+1)⁺; Retention time: 1.541 minutes; LC method F.

### Step 5: (2R)-3-(1-Methylcyclopropyl)-2-[[(1R)-1-phenylethyl]amino]propanoic acid

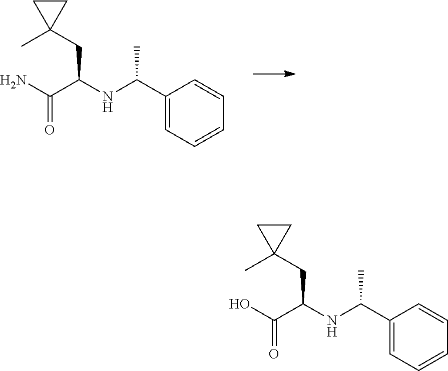 [SMILES: C[C@@H](N[C@H](CC1(C)CC1)C(N)=O)c1ccccc1 ; C[C@@H](N[C@H](CC1(C)CC1)C(=O)O)c1ccccc1]

To a solution of lithium hydroxide monohydrate (324 g, 7.7210 mol) in water (5 L) was added at 70° C. (2R)-3-(1-methylcyclopropyl)-2-[[(1R)-1-phenylethyl]amino]propanamide (hydrochloride salt) (228.9 g, 809.38 mmol). The reaction was heated to 97° C. and stirred for 68 hours. The reaction mixture was then cooled to room temperature and neutralized to pH 6 using 3M aqueous HCl and the product recovered by filtration. The product was then recrystallized four times by dissolving it in 0.5M aqueous NaOH (5 L) and neutralizing to pH 6 using 3M aqueous HCl. The product obtained was then dried in vacuo at 45° C. for 72 h and (2R)-3-(1-methylcyclopropyl)-2-[[(1R)-1-phenylethyl]amino]propanoic acid (161.5 g, 65%) was obtained as a white solid. ¹H NMR (400 MHz, DMSO-d6) δ 7.62 (d, J 6.4 Hz, 2H), 7.45-7.35 (m, 3H), 4.31 (q, J 6.4 Hz, 1H), 3.25 (dd, J 10.9, 3.8 Hz, 1H), 2.34 (dd, J 13.6, 3.5 Hz, 1H), 1.61 (d, J 6.6 Hz, 3H), 1.33 (dd, J 13.3, 11.4 Hz, 1H), 0.82 (s, 3H), 0.25-0.11 (m, 4H). 2H labile missing. ESI-MS m/z calc. 247.15723, found 248.2 (M+1)⁺; Retention time: 1.68 minutes; LC method F.

### Step 6: (2R)-3-(1-Methylcyclopropyl)-2-[[(1R)-1-phenylethyl]amino]propan-1-ol

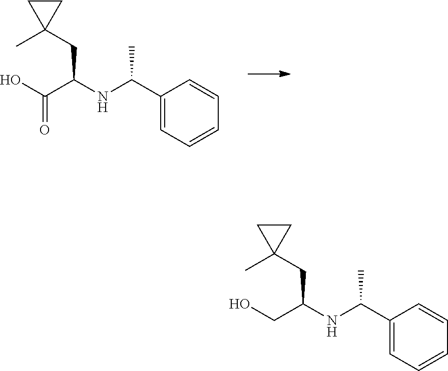 [SMILES: C[C@@H](N[C@H](CC1(C)CC1)C(=O)O)c1ccccc1 ; C[C@@H](N[C@@H](CO)CC1(C)CC1)c1ccccc1]

To a solution of (2R)-3-(1-methylcyclopropyl)-2-[[(1R)-1-phenylethyl]ammonio]propanoate (160 g, 523.26 mmol) in THE (3.2 L) was added LiAlH₄ (40 g, 1.0539 mol) over 3 hours at 20-25° C. After an additional hour of stirring at room temperature, the reaction mixture was cooled down to 10° C. and water (38.000 g, 38 mL, 2.1093 mol) was added over 150 minutes. NaOH (35 mL of 6 M, 210.00 mmol) and water (38.000 g, 38 mL, 2.1093 mol) were then successively added. The mixture was stirred overnight, at room temperature. The reaction mixture was then filtered over a bed of Celite (bottom, 80 g) and magnesium sulfate (top, 120 g). The cake was washed with THE (800 mL). The combined mother liquors were concentrated in vacuo. The resulting yellowish oil was dissolved in diethyl ether (2 L) and a solution of HCl in dioxane (130 mL of 4 M, 520.00 mmol) was added dropwise over 30 minutes inducing a precipitation. After 1 hour of stirring at 20° C., the solids were recovered by filtration, washed with diethyl ether (1 L) and dried in vacuo to afford (2R)-3-(1-methylcyclopropyl)-2-[[(1R)-1-phenylethyl]amino]propan-1-ol (hydrochloride salt) (126 g, 89%)¹H NMR (400 MHz, DMSO-d6) δ 9.30 (br. s., 1H), 9.11 (br. s., 1H), 7.69 (d, J 7.1 Hz, 2H), 7.48-7.35 (m, 3H), 5.43 (t, J 5.3 Hz, 1H), 4.56 (br. s., 1H), 3.79 (d, J 12.5 Hz, 1H), 3.65-3.52 (m, 1H), 2.89 (br. s., 1H), 1.82 (dd, J 13.7, 2.2 Hz, 1H), 1.62 (d, J 6.6 Hz, 3H), 1.26 (dd, J 13.7, 11.2 Hz, 1H), 0.72 (s, 3H), 0.41-0.27 (m, 1H), 0.24-0.06 (m, 3H). ESI-MS m z calc. 233.17796, found 234.2 (M+1)⁺; Retention time: 1.82 minutes; LC method F.

### Step 7: (2R)-2-Amino-3-(1-methylcyclopropyl)propan-1-ol

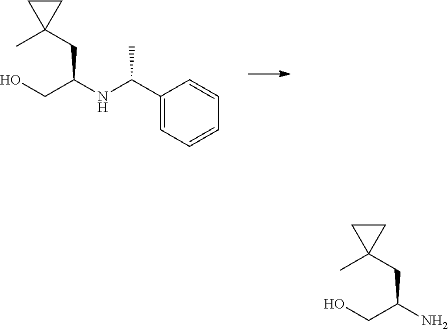

A solution of (2R)-3-(1-methylcyclopropyl)-2-[[(1R)-1-phenylethyl]amino]propan-1-ol (hydrochloride salt) (125 g, 463.29 mmol) in ethanol (1.5 L) was added to palladium on carbon (25 g, 5% w/w, 11.746 mmol). The reaction vessel was purged with nitrogen and then filled with hydrogen (75 psi) and the reaction was stirred at 50° C. for 24 h. The mixture was filtered through a Pall filter 0.45 μm, washing with EtOH (2×500 mL), and the filtrate concentrated in vacuo. The white solid obtained was triturated with MTBE (625 mL) during 1 hour and then filtered, washed with MTBE (500 mL) and dried in vacuo to afford (2R)-2-amino-3-(1-methylcyclopropyl)propan-1-ol (hydrochloride salt) (71.78 g, 93%) as a white solid. ¹H NMR (400 MHz, DMSO-d6) δ 7.98 (br. s., 3H), 5.30 (t, J 4.9 Hz, 1H), 3.69 (dt, J 11.4, 3.6 Hz, 1H), 3.47 (dt, J 11.5, 5.7 Hz, 1H), 3.29-3.16 (m, 1H), 1.62 (dd, J 13.9, 5.9 Hz, 1H), 1.35 (dd, J=13.9, 8.6 Hz, 1H), 1.01 (s, 3H), 0.41-0.19 (m, 4H). ESI-MS m/z calc. 129.11537, found 130.2 (M+1)⁺; Retention time: 0.34 minutes; LC method F.

### Example 10: Preparation of 3-[[4-[(2R)-2-amino-4-fluoro-4-methyl-pentoxy]-6-(2,6-dimethylphenyl)pyrimidin-2-yl]sulfamoyl]benzoic acid

### Step 1: Benzyl(3R)-3-(tert-butoxycarbonylamino)-4-hydroxy-butanoate

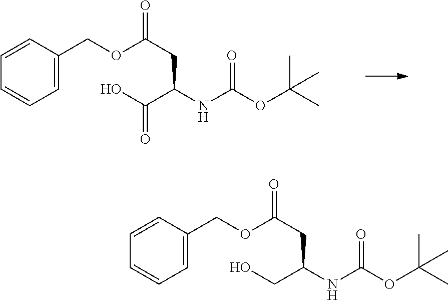 [SMILES: CC(C)(C)OC(=O)N[C@H](CC(=O)OCc1ccccc1)C(=O)O ; CC(C)(C)OC(=O)N[C@@H](CO)CC(=O)OCc1ccccc1]

A stirred solution of (2R)-4-benzyloxy-2-(tert-butoxycarbonylamino)-4-oxo-butanoic acid (20 g, 61.854 mmol) was dissolved in tetrahydrofuran (200 mL) then cooled down to −50° C. Then N-methylmorpholine (7.5440 g, 8.2 mL, 74.585 mmol) was added, followed by isobutylchloroformate (10.185 g, 9.7 mL, 74.573 mmol). The reaction was stirred at −50° C. for 2 h then the reaction was filtered, and the filtrate was cooled down to −10° C. Sodium borohydride (3.50 g, 92.513 mmol) was added and the reaction was allowed to reach room temperature and stirred at room temperature for 4 h. The reaction was quenched at 0° C. by the dropwise addition of water (200 mL). The layers were separated, and the aqueous layer was extracted with ethyl acetate (5×150 mL). The combined organic layers were washed with brine (250 mL), dried over magnesium sulfate, filtered and concentrated under reduced pressure to afford crude benzyl (3R)-3-(tert-butoxycarbonylamino)-4-hydroxy-butanoate (17.857 g, 49%) as a thick translucent oil which was used in the next step without further purification. ESI-MS m/z calc. 309.1576, found 332.2 (M+23)+; 210.2 (M-99)⁺; Retention time: 1.7 minutes; LC method I.

### Step 2: Benzyl 2-[(4R)-2-oxooxazolidin-4-yl]acetate

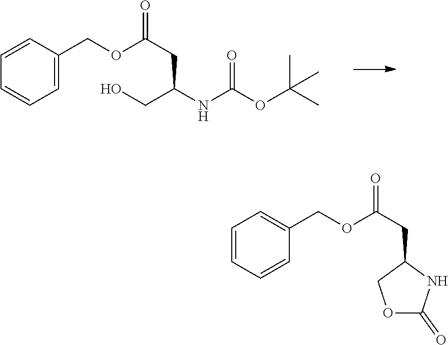 [SMILES: CC(C)(C)OC(=O)N[C@@H](CO)CC(=O)OCc1ccccc1 ; O=C(C[C@@H]1COC(=O)N1)OCc1ccccc1]

To a stirred solution of crude benzyl (3R)-3-(tert-butoxycarbonylamino)-4-hydroxy-butanoate (17.9 g, 30.667 mmol) in anhydrous 1,2-dichloroethane (140 mL) under nitrogen atmosphere at 0° C. were successively added pyridine (23.472 g, 24 mL, 296.74 mmol) and methanesulfonic anhydride (10 g, 57.407 mmol). The reaction was stirred at 0° C. for 15 minutes then stirred at room temperature for 2 h and finally stirred overnight at 90° C. The reaction was then cooled down to room temperature, diluted with dichloromethane (140 mL) and quenched by the addition of an aqueous solution of 1 N hydrochloric acid (400 mL). The layers were separated, and the aqueous layer was extracted with dichloromethane (4×100 mL). the combined organic layers were washed with brine (250 mL), dried over magnesium sulfate, filtered, and concentrated under reduced pressure to afford a yellow oil which was purified by flash chromatography on silica gel using a 120 g HP Gold column and eluting with a gradient of ethyl acetate in heptanes (15 to 100% in 15 CV). The desired fractions were concentrated under reduced pressure, dried under vacuum to afford benzyl 2-[(4R)-2-oxooxazolidin-4-yl]acetate (5.01 g, 68%) as an off-white powder. ¹H NMR (400 MHz, CDCl₃) δ 7.48-7.30 (m, 5H), 5.49 (br. s, 1H), 5.16 (s, 2H), 4.56 (t, J 8.6 Hz, 1H), 4.32-4.18 (m, 1H), 4.06 (dd, J 8.9, 5.7 Hz, 1H), 2.78-2.62 (m, 2H). ESI-MS m/z calc. 235.08446, found 236.2 (M+1)⁺; Retention time: 1.51 minutes; LC method I.

### Step 3: (4R)-4-(2-Hydroxy-2-methyl-propyl)oxazolidin-2-one

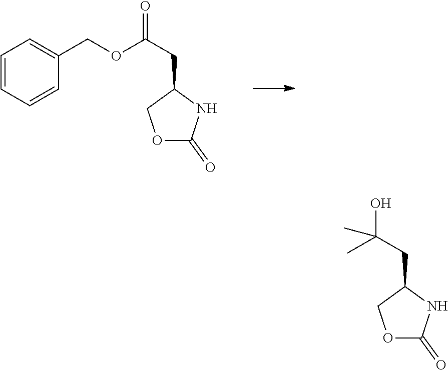 [SMILES: O=C(C[C@@H]1COC(=O)N1)OCc1ccccc1 ; CC(C)(O)C[C@@H]1COC(=O)N1]

A flask was flame-dried then cooled down to room temperature with a nitrogen stream, then charged with anhydrous toluene (30 mL) and anhydrous tetrahydrofuran (30 mL). The solvent mixture was then cooled down to −50° C. A solution of methyl magnesium bromide in diethylether (29 mL of 3 M, 87.000 mmol) was canulated to the mixture and stirred for 30 minutes at −50° C. after which time a solution of benzyl 2-[(4R)-2-oxooxazolidin-4-yl]acetate (5.01 g, 18.678 mmol) in anhydrous tetrahydrofuran (15 mL) was canulated. The reaction was stirred for 30 minutes at −50° C. then allowed to reach room temperature and stirred overnight at room temperature. The reaction was then cooled down to 0° C. and quenched by the dropwise addition of a solution of acetic acid (7.9200 g, 7.5 mL, 131.89 mmol) in water (20 mL). The reaction mixture was vigorously stirred at room temperature for 1 h. Then sodium chloride was added to saturate the aqueous layer. The reaction was then dried over sodium sulfate and filtered on celite pad. The cake was washed with dichloromethane (5×100 mL) and the filtrate was concentrated under reduced pressure to afford a yellow oil which was purified by flash chromatography on silica gel using a 120 g HP Gold column and eluting with a gradient of isopropanol in dichloromethane (0 to 6% in 20 CV). The desired fractions were concentrated under reduced pressure, dried under high vacuum to afford (4R)-4-(2-hydroxy-2-methyl-propyl)oxazolidin-2-one (2.02 g, 65%) as pale yellow crystalline solid. ¹H NMR (400 MHz, DMSO-d6) δ 7.34 (br. s, 1H), 4.50-4.26 (m, 2H), 4.06-3.88 (m, 2H), 1.78-1.48 (m, 2H), 1.10 (s, 6H). ESI-MS m/z calc. 159.08954, found 160.2 (M+1)⁺; Retention time: 0.74 minutes; LC method I.

### Step 4: (4R)-4-(2-Fluoro-2-methyl-propyl)oxazolidin-2-one

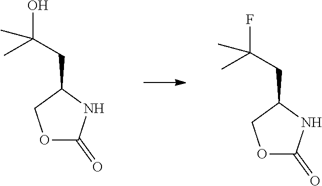

To a stirred solution of (diethylamino)sulfur trifluoride (4.1480 g, 3.4 mL, 25.734 mmol) in anhydrous dichloromethane (70 mL) at −78° C. was canulated a solution of (4R)-4-(2-hydroxy-2-methyl-propyl)oxazolidin-2-one (4.29 g, 25.603 mmol) in anhydrous dichloromethane (25 mL). The resulting solution was stirred for 15 min at −78° C. then allowed to reach room temperature and stirred at room temperature for 2 h. The reaction was then slowly added to a solution of saturated aqueous sodium bicarbonate (500 mL) at 0° C. The solution was then vigorously stirred for 30 minutes at room temperature. The layers were separated, and the aqueous layer was extracted with dichloromethane (4×150 mL). The combined organic layers were washed with water (200 mL), brine (200 mL), dried over magnesium sulfate, filtered and concentrated under reduced pressure to afford crude (4R)-4-(2-fluoro-2-methyl-propyl)oxazolidin-2-one (3.51 g, 81%) as brown crystals which were directly used in the next step without further purification. ¹H NMR (400 MHz, DMSO-d6) δ 7.56 (br. s, 1H), 4.44 (td, J 8.2, 1.0 Hz, 1H), 4.06-3.97 (m, 1H), 3.95-3.88 (m, 1H), 1.93-1.80 (m, 2H), 1.35 (s, 3H), 1.30 (s, 3H). ESI-MS m/z calc. 161.0852, found 162.2 (M+1)⁺; Retention time: 1.28 minutes; LC method I.

### Step 5: (2R)-2-Amino-4-fluoro-4-methyl-pentan-1-ol

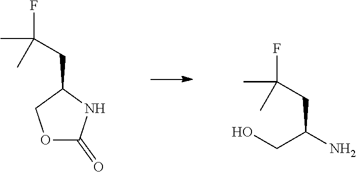

To a solution of potassium hydroxide (2.5 g, 44.559 mmol) in ethanol (15 mL) and water (1.5 mL) was added (4R)-4-(2-fluoro-2-methyl-propyl)oxazolidin-2-one (2.3 g, 13.557 mmol). The reaction mixture was heated at 100° C. for 4 h. The reaction was then allowed to reach room temperature and concentrated under reduced pressure. The residue was co-evaporated with toluene (3×10 mL) to afford a pale orange residue which was filtered on a celite pad, washed with dichloromethane (3×20 mL). The filtrate was concentrated under reduced pressure to afford (2R)-2-amino-4-fluoro-4-methyl-pentan-1-ol (1.82 g, 94%) as a dark orange oil which was directly used in the next step without further purification ¹H NMR (400 MHz, CD₃OD) δ 3.50 (dd, J 10.6, 4.8 Hz, 1H), 3.35-3.27 (m, 1H), 3.17-3.09 (m, 1H), 1.77-1.59 (m, 2H), 1.42 (s, 3H), 1.37 (s, 3H).19F NMR (377 MHz, CD₃OD) δ −139.34 (s, 1F). ESI-MS m/z calc. 135.10594, found 136.2 (M+1)⁺; Retention time: 0.23 minutes; LC method I.

### Step 6: 3-[[4-[(2R)-2-Amino-4-fluoro-4-methyl-pentoxy]-6-(2,6-dimethylphenyl)pyrimidin-2-yl]sulfamoyl]benzoic acid

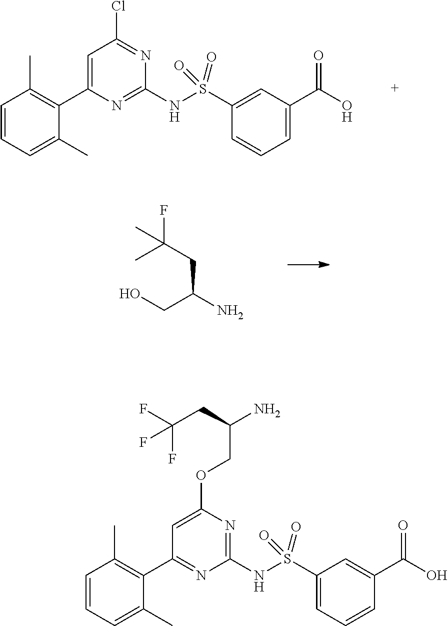

3-[[4-Chloro-6-(2,6-dimethylphenyl)pyrimidin-2-yl]sulfamoyl]benzoic acid (1 g, 2.393 mmol) and (2R)-2-amino-4-fluoro-4-methyl-pentan-1-ol (391 mg, 2.892 mmol) were combined under nitrogen in anhydrous THE (9 mL). To the resulting cloudy solution sodium tert-butoxide (1.05 g, 10.93 mmol) was added in one portion resulting in the dissolution of the solids and a slightly exothermic reaction. The mixture was stirred at room temperature for 2.5 h. The reaction was diluted with ethyl acetate (20 mL), HCl (20 mL of 1 M, 20.00 mmol) and brine (20 mL) and the resulting two phases were separated. The aqueous phase was further extracted with EtOAc (3×15 mL). The combined organic extracts were dried over sodium sulfate and concentrated. The residue was triturated in a mixture of EtOAc and hexanes (1:3, v:v) and the resulting suspension was stirred at rt overnight. The solid was filtered and dried to give 3-[[4-[(2R)-2-amino-4-fluoro-4-methyl-pentoxy]-6-(2,6-dimethylphenyl)pyrimidin-2-yl]sulfamoyl]benzoic acid (hydrochloride salt) (1.238 g, 94%) as a tan solid. ESI-MS m/z calc. 516.18427, found 517.45 (M+1)⁺; Retention time: 0.99 minutes; LC method A.

### Example 11: Preparation of 3-[[4-[(2R)-2-amino-5-methyl-hexoxy]-6-(2,6-dimethylphenyl)pyrimidin-2-yl]sulfamoyl]benzoic acid

### Step 1: (2R)-2-Amino-5-methyl-hexan-1-ol

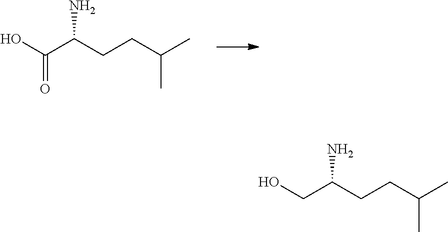

Borane tetrahydrofuran complex in THF (58 mL of 1 M, 58.000 mmol) was slowly added to a suspension of (2R)-2-amino-5-methyl-hexanoic acid (4.05 g, 27.893 mmol) in 2-methyltetrahydrofuran (40 mL). The reaction was stirred at room temperature for 16 h. Aqueous HCl (28 mL of 3 M, 84.00 mmol) was added keeping the temperature under 25° C. and the reaction was stirred at room temperature for 45 minutes. MeTHF (100 mL) was added and the excess THE was removed by evaporation. The solution was basified, at pH around 9, with NaOH 25% aqueous solution (10 mL). The organic phase was separated. The aqueous layer was extracted with MeTHF (2×50 mL). The combined organic layers were washed with brine (50 mL), dried over anhydrous sodium sulfate, filtered, and concentrated under vacuum. Aqueous HCl (14 mL of 3 M, 42.000 mmol) was added to the residue, water was evaporated to dryness and then co-evaporated with isopropanol (3×50 mL). MTBE (100 mL) was added to the residue and the solvent was evaporated to dryness to afford (2R)-2-amino-5-methyl-hexan-1-ol (hydrochloride salt) (2.231 g, 48%) as a white solid. ¹H NMR (400 MHz, DMSO-d6) δ 7.82 (br. s., 3H), 5.26 (t, J 4.9 Hz, 1H), 3.58 (dt, J 11.2, 4.4 Hz, 1H), 3.42 (dt, J 11.3, 5.7 Hz, 1H), 3.00 (br. s., 1H), 1.57-1.43 (m, 3H), 1.25-1.15 (m, 2H), 0.86 (d, J 6.1 Hz, 6H). ESI-MS m/z calc. 131.131, found 132.2 (M+1)⁺; Retention time: 2.022 minutes. To the aqueous phase, NaOH 25% aqueous (3 mL) was added and the aqueous layer was extracted with MeTHF (2×50 mL). The combined organic layers were washed with brine (50 mL), dried over anhydrous sodium sulfate, filtered, and concentrated under vacuum. Aqueous HCl (14 mL of 3 M, 42.000 mmol) was added to the residue, water was evaporated to dryness and then co-evaporated with isopropanol (3×50 mL). MTBE (100 mL) was added to the residue and the solvent was evaporated to dryness to afford a second batch of (2R)-2-amino-5-methyl-hexan-1-ol (hydrochloride salt) (2.227 g, 45%) as a white solid. ¹H NMR (400 MHz, DMSO-d6) δ 7.94 (br. s., 3H), 5.27 (t, J 5.0 Hz, 1H), 3.58 (dt, J 11.4, 4.3 Hz, 1H), 3.43 (dt, J 11.5, 5.7 Hz, 1H), 3.06-2.93 (m, 1H), 1.58-1.43 (m, 3H), 1.25-1.15 (m, 2H), 0.86 (d, J 6.4 Hz, 6H). ESI-MS m/z calc. 131.131, found 132.2 (M+1)⁺; Retention time: 1.994 minutes. Total quantity of 4.458 g and total yield of 95%. LC method K.

### Step 2: 3-[[4-[(2R)-2-Amino-5-methyl-hexoxy]-6-(2,6-dimethylphenyl)pyrimidin-2-yl]sulfamoyl]benzoic acid

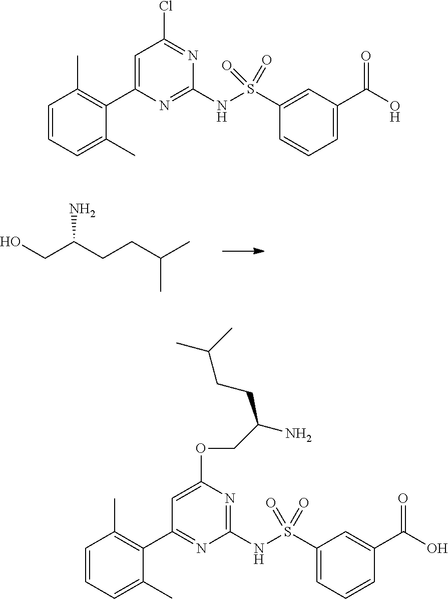

3-[[4-Chloro-6-(2,6-dimethylphenyl)pyrimidin-2-yl]sulfamoyl]benzoic acid (1.1 g, 2.632 mmol) and (2R)-2-amino-5-methyl-hexan-1-ol (hydrochloride salt) (538 mg, 3.209 mmol) were combined under nitrogen in anhydrous THF (10 mL). To the resulting cloudy solution sodium tert-butoxide (1.01 g, 10.51 mmol) was added in one portion resulting in fast dissolution of the solids and a slightly exothermic reaction. The mixture was stirred at room temperature for 1 h. More (2R)-2-amino-5-methyl-hexan-1-ol (hydrochloride salt) (86 mg, 0.5129 mmol) and sodium tert-butoxide (Sodium salt) (140 mg, 1.457 mmol) were added and the mixture was stirred at rt for 1.5 h. The reaction was diluted with ethyl acetate (20 mL), HCl (20 mL of 1 M, 20.00 mmol) and brine (20 mL) and the resulting two phases were separated. The aqueous phase was further extracted with EtOAc (3×15 mL). The combined organic extracts were dried over sodium sulfate and concentrated. The residue was triturated in a mixture of EtOAc and hexanes (1:3, v:v) and the resulting suspension was stirred at rt for 1 h. The solid was filtered and dried to give 3-[[4-[(2R)-2-amino-5-methyl-hexoxy]-6-(2,6-dimethylphenyl)pyrimidin-2-yl]sulfamoyl]benzoic acid (hydrochloride salt) (1.32 g, 82%) as an off-white solid. ESI-MS m z calc. 512.20935, found 513.59 (M+1)⁺; Retention time: 1.1 minutes; LC method A.

### Example 12: Preparation of 3-[[4-[(2R)-2-amino-4-cyclopropyl-butoxy]-6-(2,6-dimethylphenyl)pyrimidin-2-yl]sulfamoyl]benzoic acid

### Step 1: Methyl (2R)-2-(benzyloxycarbonylamino)hex-5-enoate

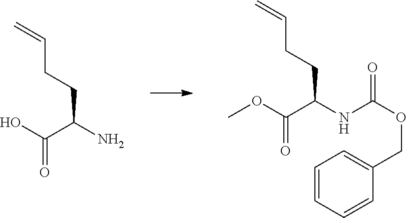

(2R)-2-Aminohex-5-enoic acid (2 g, 15.485 mmol) was mixed in MeOH (40 mL) and cooled in a ˜−10° C. acetone dry ice bath. thionyl chloride (4.0775 g, 2.5 mL, 34.273 mmol) was added dropwise. The clear mixture was then let reach rt and stirred for 24 h. It was then concentrated. The off-white solid obtained was taken into DCM (30 mL) and Water (15 mL) and cooled in ice water bath. Sodium bicarbonate (8.6 g, 102.37 mmol) was added, followed by CbzOSu (4.68 g, 18.779 mmol). The yellowish mixture was stirred efficiently for 15 h (ice bath temperature reaching rt during the course). DCM and water (50 ml each) were added. Layers were separated. The DCM solution was dried over anhydrous magnesium sulfate, filtered and concentrated. The residue was purified by silica gel chromatography (80 g column), using 5-40% EtOAc in Hexanes, to afford methyl (2R)-2-(benzyloxycarbonylamino)hex-5-enoate (4.23 g, 94%) as colorless oil. ESI-MS m/z calc. 277.1314, found 278.3 (M+1)⁺; Retention time: 2.86 minutes; LC method E.

### Step 2: Methyl (2R)-2-(benzyloxycarbonylamino)-4-cyclopropyl-butanoate

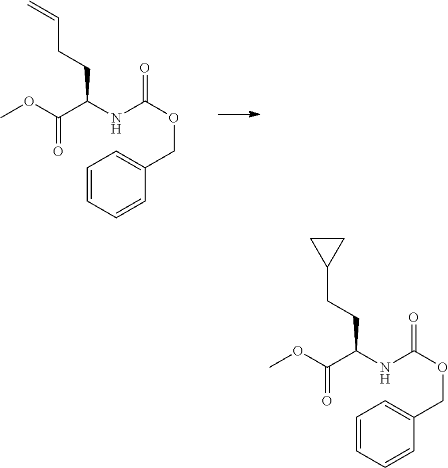 [SMILES: C=CCC[C@@H](NC(=O)OCc1ccccc1)C(=O)OC ; COC(=O)[C@@H](CCC1CC1)NC(=O)OCc1ccccc1]

Et₂Zn in hexanes (50 mL of 1 M, 50.000 mmol) was diluted with DCM (25 mL) and cooled to ˜−10° C. TFA (5.7720 g, 3.9 mL, 50.621 mmol) in DCM (10 mL) was added dropwise. The mixture was stirred at <0° C. for 15 min. CH₂I₂ (12.968 g, 3.9 mL, 48.418 mmol) in DCM (25 mL) was added in portions. The mixture was stirred at the same temperature for 15 min. Methyl (2R)-2-(benzyloxycarbonylamino)hex-5-enoate (3.9 g, 13.360 mmol) in DCM (25 mL) was then added in portions. The mixture was stirred for 15 h (reaching rt gradually). HCl (0.2 N aqueous) was added in portions (40 ml total). More DCM (60 ml) was added. Layers were separated. The organic layer was washed with brine, dried over anhydrous magnesium sulfate, filtered and concentrated. The residue oil was purified by silica gel chromatography (80 g column), using 0-40% EtOAc in Hexanes, to afford methyl (2R)-2-(benzyloxycarbonylamino)-4-cyclopropyl-butanoate (3.65 g, 89%) as colorless oil. ¹H NMR (500 MHz, Chloroform-d) δ 7.43-7.28 (m, 5H), 5.31-5.20 (m, 1H), 5.11 (s, 2H), 4.49-4.35 (m, 1H), 3.74 (s, 3H), 2.02-1.86 (m, 1H), 1.83-1.67 (m, 1H), 1.32-1.19 (m, 2H), 0.77-0.59 (m, 1H), 0.52-0.35 (m, 2H), 0.09-−0.04 (m, 2H). ESI-MS m/z calc. 291.14706, found 292.5 (M+1)⁺; Retention time: 3.01 minutes; LC method E.

### Step 3: Benzyl N-[(1R)-3-cyclopropyl-1-(hydroxymethyl)propyl]carbamate

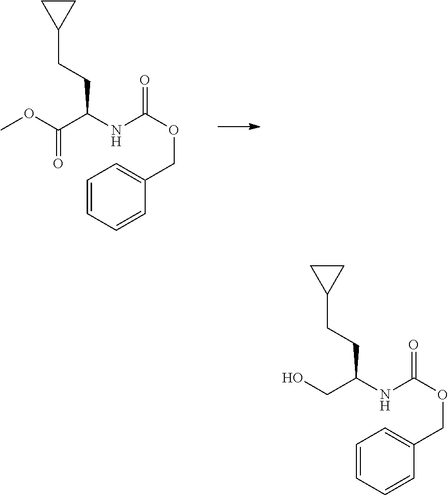 [SMILES: COC(=O)[C@@H](CCC1CC1)NC(=O)OCc1ccccc1 ; O=C(N[C@@H](CO)CCC1CC1)OCc1ccccc1]

Methyl (2R)-2-(benzyloxycarbonylamino)-4-cyclopropyl-butanoate (3.94 g, 12.847 mmol) was dissolved in THE (40 mL) and the solution was cooled in ice water bath and stirred under a nitrogen balloon. LiBH₄ in THE (12 mL of 2 M, 24.000 mmol) was added in small portions over 10 min. The ice bath was removed and the mixture was stirred at rt for 2 h. NH₄Cl (20 ml, saturated aqueous) was added, followed by EtOAc (50 ml) and water (40 ml). Layers were separated. The organic layer was washed with more water (30 ml×2), brine, dried over anhydrous magnesium sulfate, filtered and concentrated to afford crude benzyl N-[(1R)-3-cyclopropyl-1-(hydroxymethyl)propyl]carbamate (3.75 g, 100%) as colorless oil. ESI-MS m z calc. 263.15213, found 264.4 (M+1)⁺; Retention time: 2.65 minutes; LC method E.

### Step 4: (2R)-2-Amino-4-cyclopropyl-butan-1-ol

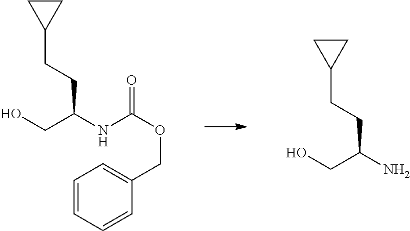 [SMILES: O=C(N[C@@H](CO)CCC1CC1)OCc1ccccc1 ; N[C@@H](CO)CCC1CC1]

Benzyl N-[(1R)-3-cyclopropyl-1-(hydroxymethyl)propyl]carbamate (3.75 g, 12.817 mmol) was dissolved in EtOH (60 mL). HCl aqueous (12.9 mL of 1 M, 12.900 mmol) was added, followed by Pd on activated carbon (300 mg, 5% w/w, 0.1410 mmol). The mixture was vacuumed and refilled with H₂ balloon and stirred at rt for 4 h. It was then filtered through a celite pad and washed with MeOH. The combined filtrate was concentrated to give (2R)-2-amino-4-cyclopropyl-butan-1-ol (hydrochloride salt) (2.15 g, 96%) as a colorless oil. ESI-MS m z calc. 129.11537, found 130.4 (M+1)⁺; Retention time: 1.09 minutes; LC method E.

### Step 5: 3-[[4-[(2R)-2-Amino-4-cyclopropyl-butoxy]-6-(2,6-dimethylphenyl)pyrimidin-2-yl]sulfamoyl]benzoic acid

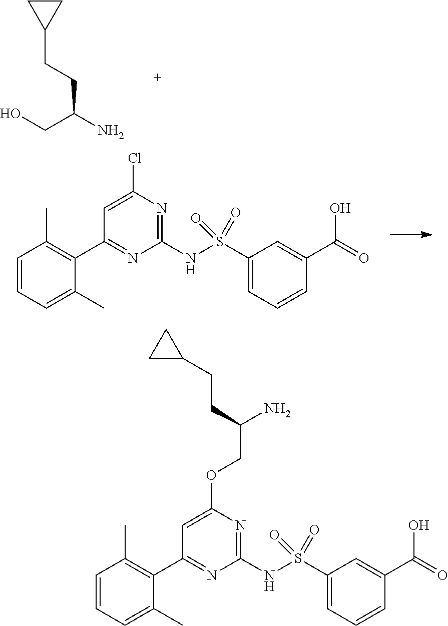

3-[[4-chloro-6-(2,6-Dimethylphenyl)pyrimidin-2-yl]sulfamoyl]benzoic acid (3 g, 7.1793 mmol) and (2R)-2-amino-4-cyclopropyl-butan-1-ol (hydrochloride salt) (2.15 g, 12.329 mmol) were mixed in THE (20 mL) at rt. Sodium t-butoxide (2.8 g, 29.135 mmol) was added in one portion. The mixture was stirred at rt for 1 h. More sodium t-butoxide (1.4 g, 14.568 mmol) was added. The mixture was stirred at rt for 2 h. HCl aqueous (60 mL of 1 M, 60.000 mmol) was added, followed by EtOAc (60 ml). Layers were separated. The aqueous layer was extracted with more EtOAc (20 ml). The combined EtOAc solution was washed with brine, dried over anhydrous magnesium sulfate, filtered and concentrated. The residue was retaken into EtOAc (˜20 ml) and sonicated briefly. The supernatant was discarded. The precipitate was dissolved in THE and transferred into shipment vial and dried under high vacuum for 20 h to afford 3-[[4-[(2R)-2-amino-4-cyclopropyl-butoxy]-6-(2,6-dimethylphenyl)pyrimidin-2-yl]sulfamoyl]benzoic acid (hydrochloride salt) (4.14 g, 95%) as slightly yellowish solid. ¹H NMR (500 MHz, DMSO-d6) δ 13.20 (s, 1H), 8.44 (s, 1H), 8.28-8.05 (m, 5H), 7.69 (t, J 0.8, 7.8 Hz, 1H), 7.25 (t, J 7.6, 7.6 Hz, 1H), 7.12 (d, J 7.6 Hz, 2H), 6.30 (s, 1H), 4.36 (dd, J 11.8, 3.3 Hz, 1H), 4.21 (dd, J 11.8, 6.6 Hz, 1H), 3.57-3.48 (m, 1H), 1.99 (d, J 8.5 Hz, 6H), 1.72-1.63 (m, 2H), 1.33-1.19 (m, 2H), 0.77-0.61 (m, 1H), 0.47-0.34 (m, 2H), 0.08-−0.01 (m, 2H). ESI-MS m/z calc. 510.1937, found 511.8 (M+1)⁺; Retention time: 1.78 minutes; LC method H.

### Example 13: Preparation of 3-[[[4-[(2R)-2-amino-4,4-dimethyl-pentoxy]-6-(2,6-dimethylphenyl)pyrimidin-2-yl]amino]sulfonimidoyl]benzoic acid isomer A

### Step 1: Methyl 3-[[4-chloro-6-(2,6-dimethylphenyl)pyrimidin-2-yl]amino]sulfanylbenzoate

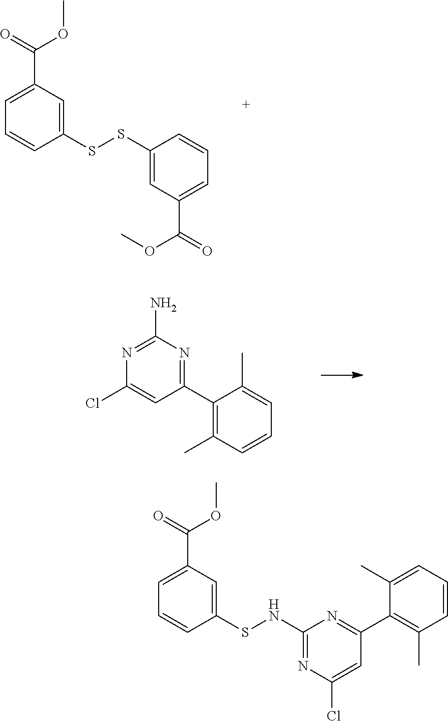

A 500 ml single necked round bottomed flask was charged, under an atmosphere of N₂, with methyl 3-[(3-methoxycarbonylphenyl)disulfanyl]benzoate (26.5 g, 79.244 mmol), dichloromethane (167 mL) and pyridine (2.9340 g, 3 mL, 37.092 mmol). To the resulting amber solution was added dropwise sulfuryl chloride (10.7 g, 79.277 mmol) (no exotherm observed). The solution turned deep orange and was stirred 10 minutes at room temperature. In another 2 L three necked round bottomed flask was charged, under an atmosphere of N₂, with 4-chloro-6-(2,6-dimethylphenyl)pyrimidin-2-amine (25.95 g, 111.04 mmol) and dichloromethane (618 mL). The resulting light-yellow solution was cooled to 2° C. (internal temperature) using an ice bath. Then, triethylamine (47.335 g, 65.2 mL, 467.78 mmol) was added dropwise, keeping the internal temperature below 10° C. Once this solution reached again 2° C., the first solution was added dropwise (exothermic), keeping the internal temperature below 10° C. The resulting pale orange suspension was stirred at 2° C. (ice bath not removed) during 1 h. The reaction was poured into 5% wt. aqueous solution of sodium bicarbonate (585 ml, 63 vol.). After phases separation, the aqueous phase was extracted with DCM (3×50 ml). The combined organic phases were dried over sodium sulfate, filtered, and concentrated to dryness to afford a crude product (76.54 g) as amber viscous oil. The oil was mixed with silica gel (80 g, 1 part vs crude) and DCM. The suspension was concentrated to dryness to afford a fine orange powder. This dry pack was loaded onto silica gel (460 g, 6 parts vs crude) packed with Heptane, in a fritted glass. Elution was started with Heptane/EtOAc (80/20) (1 L) followed by 70/30 (5 L). The filtrate (pale yellow) was concentrated to dryness. During the concentration, a fine white solid was formed. The solid was suspended into Heptane/EtOAc (95/5) (50 ml), was cooled in an ice bath and was filtered. The off-white solid was washed with cold Heptane/EtOAc (95/5) (50 ml) and was dried under high vacuum to furnish methyl 3-[[4-chloro-6-(2,6-dimethylphenyl)pyrimidin-2-yl]amino]sulfanylbenzoate (26.46 g, 83%) as white powder. ¹H NMR (400 MHz, CDCl₃) δ 7.90-7.87 (m, 1H), 7.84 (dt, J 7.3, 1.6 Hz, 1H), 7.46-7.33 (m, 2H), 7.22-7.12 (m, 1H), 7.09-6.99 (m, 3H), 6.80 (s, 1H), 3.90 (s, 3H), 2.05 (s, 6H). ESI-MS m/z calc. 399.0808, found 400.0 (M+1)⁺; Retention time: 2.018 minutes; LC method I.

### Step 2: Methyl 3-[4-chloro-6-(2,6-dimethylphenyl)pyrimidin-2-yl]sulfinamoylbenzoate

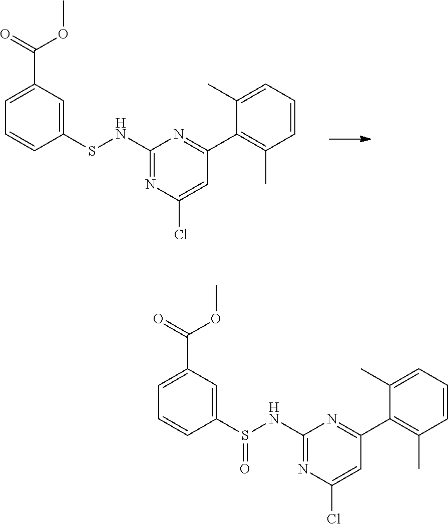 [SMILES: COC(=O)c1cccc(SNc2nc(Cl)cc(-c3c(C)cccc3C)n2)c1 ; COC(=O)c1cccc(S(=O)Nc2nc(Cl)cc(-c3c(C)cccc3C)n2)c1]

A 500 ml three necked round bottomed flask, equipped with an internal temperature probe, was charged, under an atmosphere of N₂, with methyl 3-[[4-chloro-6-(2,6-dimethylphenyl)pyrimidin-2-yl]amino]sulfanylbenzoate (26 g, 65.017 mmol) and dichloromethane (624 mL). The resulting pale-yellow solution was cooled to 2° C. (internal temperature) using an ice bath. Then, 3-chloroperbenzoic acid (15.6 g, 69.608 mmol) was added portion wise (light exotherm) keeping the internal temperature below 5° C. The resulting light-yellow suspension was stirred 1 h at 2° C. To the reaction mixture was added 5% wt. aqueous solution of Na₂S₂O₃ (520 ml, 20 vol.). An exotherm was observed and internal temperature reached 10° C. The mixture was poured into 5% wt. aqueous sodium bicarbonate (520 ml, 20 vol.). After phases separation, the aqueous phase was extracted with DCM (3×100 ml). Combined organic phases were dried over sodium sulfate, concentrated to dryness to afford crude product as yellow oil. The oil was mixed with Heptane/EtOAc (95/5) (200 ml) and was sonicated to obtain a white slurry. The slurry was stirred 30 minutes at room temperature. The solid was recovered by filtration, washed with cold Heptane/EtOAc (95/5) (100 ml) and dried under high vacuum to afford methyl 3-[4-chloro-6-(2,6-dimethylphenyl)pyrimidin-2-yl]sulfinamoylbenzoate (22.76 g, 84%) as white powder. ¹H NMR (400 MHz, CDCl₃) δ 8.48 (t, J 1.7 Hz, 1H), 8.25 (dt, J 7.7, 1.4 Hz, 1H), 8.07 (dt, J 7.8, 1.5 Hz, 1H), 7.67 (t, J 7.7 Hz, 1H), 7.35 (s, 1H), 7.26-7.21 (m, 1H), 7.12 (d, J 7.6 Hz, 2H), 6.96 (s, 1H), 3.96 (s, 3H), 2.15 (s, 6H). ESI-MS m/z calc. 415.07574, found 416.0 (M+1)⁺; Retention time: 1.906 minutes; LC method I.

### Step 3: Methyl 3-[[[4-chloro-6-(2,6-dimethylphenyl)pyrimidin-2-yl]amino]sulfonimidoyl]benzoate

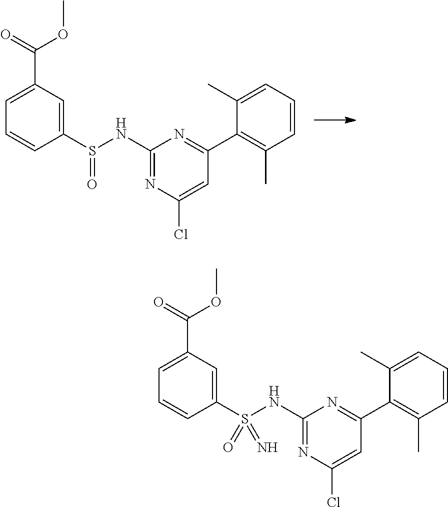 [SMILES: COC(=O)c1cccc(S(=O)Nc2nc(Cl)cc(-c3c(C)cccc3C)n2)c1 ; COC(=O)c1cccc(S(=N)(=O)Nc2nc(Cl)cc(-c3c(C)cccc3C)n2)c1]

A 3 L three necked round bottomed flask, equipped with a dropping funnel and an internal temperature probe, was charged, under an atmosphere of N₂, with methyl 3-[4-chloro-6-(2,6-dimethylphenyl)pyrimidin-2-yl]sulfinamoylbenzoate (22.5 g, 54.100 mmol) and dichloromethane (833 mL). To the resulting light-yellow solution was added portion wise 1-chloropyrrolidine-2,5-dione (10.11 g, 75.712 mmol). The milky white mixture was stirred at room temperature during 7 h. Then, the reaction was cooled to 0° C. (ice bath) and ammonia (0.4M in dioxane) (1.2 L of 0.4 M, 480.00 mmol) was added dropwise over 35 minutes. The reaction was stirred at room temperature overnight. The reaction was poured into a mixture of 5% wt. aqueous sodium bicarbonate/brine 1:1 (1 L). After phases separation, the aqueous phase was extracted with DCM (3×150 ml). Combined organic phases were washed with brine (250 ml), dried over sodium sulfate, filtered, and concentrated to dryness to afford crude product as yellow oil. The oil was solubilized in EtOAc and concentrated to dryness. A mixture of Hept/EtOAc 95/5 was added (a white solid appeared) and the solvents were concentrated to dryness. The solid was triturated with Hept/EtOAc 95/5 (200 ml). The white solid was recovered by filtration to afford methyl 3-[[[4-chloro-6-(2,6-dimethylphenyl)pyrimidin-2-yl]amino]sulfonimidoyl]benzoate (23.399 g, 81%) as an off-white powder containing about 15% wt of succinimide according to ¹H NMR. Methyl 3-[[[4-chloro-6-(2,6-dimethylphenyl)pyrimidin-2-yl]amino]sulfonimidoyl]benzoate (23.399 g, 49.577 mmol) was dissolved by sonication at 40° C. in EtOAc (250 mL). The organic phase was washed with sodium bicarbonate aqueous saturated (2×100 mL). The aqueous phase was backwashed with EtOAc (100 mL). The combined organic phases were washed with brine (100 mL), dried with magnesium sulfate, filtered and concentrated in vacuo to afford methyl 3-[[[4-chloro-6-(2,6-dimethylphenyl)pyrimidin-2-yl]amino]sulfonimidoyl]benzoate (21.98 g, 97%) as a pale yellow solid. ¹H NMR (400 MHz, CDCl₃) δ 8.69 (t, J 1.8 Hz, 1H), 8.28 (ddd, J 8.0, 1.9, 1.1 Hz, 1H), 8.23 (dt, J 7.9, 1.3 Hz, 1H), 7.56 (t, J 7.8 Hz, 1H), 7.21-7.15 (m, 1H), 7.04 (d, J 7.6 Hz, 2H), 6.74 (s, 1H), 6.00 (br. s., 2H), 3.90 (s, 3H), 2.02-1.84 (m, 6H). ESI-MS m/z calc. 430.08664, found 431.1 (M+1)⁺; Retention time: 3.975 minutes; LC method J.

### Step 4: Methyl 3-[[[4-chloro-6-(2,6-dimethylphenyl)pyrimidin-2-yl]amino]sulfonimidoyl]benzoate, isomer A, and methyl 3-[[[4-chloro-6-(2,6-dimethylphenyl)pyrimidin-2-yl]amino]sulfonimidoyl]benzoate, isomer B

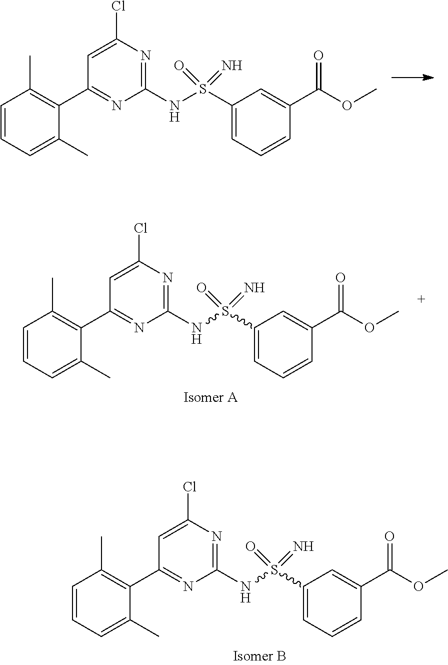

Racemic methyl 3-[[[4-chloro-6-(2,6-dimethylphenyl)pyrimidin-2-yl]amino]sulfonimidoyl]benzoate (21.98 g, 47.948 mmol) was dissolved in a 1:1 mixture of MeOH/MeCN (concentration of 1.2 g/25 mL) and submitted to chiral SFC separation (Flow rate: 75 mL/min, 15% MeOH, column: Cellulose 1, temperature=40° C., outlet pressure: 100 bar, injection volume: 600 L). Fastest eluting peak by SFC gave after evaporation to dryness and co-evaporation with 2-methyltetrahydrofuran methyl 3-[[[4-chloro-6-(2,6-dimethylphenyl)pyrimidin-2-yl]amino]sulfonimidoyl]benzoate isomer A (8.38 g, 78%) as a pale yellow solid. ¹H NMR (400 MHz, DMSO-d6) δ 8.39 (t, J 1.6 Hz, 1H), 8.15-8.07 (m, 2H), 7.89 (s, 2H), 7.67 (t, J 7.8 Hz, 1H), 7.22-7.15 (m, 1H), 7.03 (d, J 7.6 Hz, 2H), 6.91 (s, 1H), 3.83 (s, 3H), 1.93-1.51 (m, 6H). ESI-MS m/z calc. 430.0866, found 431.1 (M+1)⁺; Retention time: 3.99 minutes. The slowest eluting peak by SFC gave after evaporation to dryness and co-evaporation with 2-methyltetrahydrofuran methyl 3-[[[4-chloro-6-(2,6-dimethylphenyl)pyrimidin-2-yl]amino]sulfonimidoyl]benzoate isomer B (8.52 g, 76%) as a pale yellow solid. ¹H NMR (400 MHz, DMSO-d6) δ 8.38 (t, J 1.6 Hz, 1H), 8.14-8.07 (m, 2H), 7.89 (s, 2H), 7.67 (t, J 7.8 Hz, 1H), 7.21-7.15 (m, 1H), 7.03 (d, J 7.6 Hz, 2H), 6.91 (s, 1H), 3.83 (s, 3H), 1.89-1.59 (m, 6H). ESI-MS m/z calc. 430.0866, found 431.1 (M+1)⁺; Retention time: 3.99 minutes; LC method J.

### Step 5: 3-[[[4-Chloro-6-(2,6-dimethylphenyl)pyrimidin-2-yl]amino]sulfonimidoyl]benzoic acid isomer A

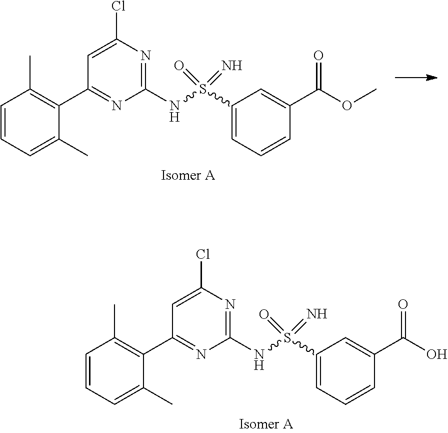

To a solution of methyl 3-[[[4-chloro-6-(2,6-dimethylphenyl)pyrimidin-2-yl]amino]sulfonimidoyl]benzoate isomer A (8.38 g, 18.125 mmol) in tetrahydrofuran (170 mL) and water (170 mL) at 0° C. was added lithium hydroxide hydrate (1.9 g, 45.277 mmol). The resulting light-yellow solution was stirred at room temperature for 16 h. The reaction mixture was diluted with aqueous saturated NH₄Cl (250 mL) and some HCl 1 N to reach pH=4. The product was extracted with EtOAc (3×150 mL) and the combined organic phases were washed with brine (200 mL), dried with magnesium sulfate, filtered and concentrated to dryness to afford 3-[[[4-chloro-6-(2,6-dimethylphenyl)pyrimidin-2-yl]amino]sulfonimidoyl]benzoic acid isomer A (7.82 g, 96%) as a pale beige solid. ¹H NMR (400 MHz, DMSO-d6) δ 8.44 (br. s., 1H), 8.11 (br. s., 1H), 7.93 (d, J 7.6 Hz, 1H), 7.72 (br. s., 2H), 7.53 (t, J 7.6 Hz, 1H), 7.21-7.11 (m, 1H), 7.02 (d, J 6.8 Hz, 2H), 6.88 (s, 1H), 1.77 (br. s., 6H). ESI-MS m/z calc. 416.07098, found 417.1 (M+1)⁺; Retention time: 3.57 minutes; LC method J.

### Step 6: 3-[[[4-[(2R)-2-Amino-4,4-dimethyl-pentoxy]-6-(2,6-dimethylphenyl)pyrimidin-2-yl]amino]sulfonimidoyl]benzoic acid isomer A

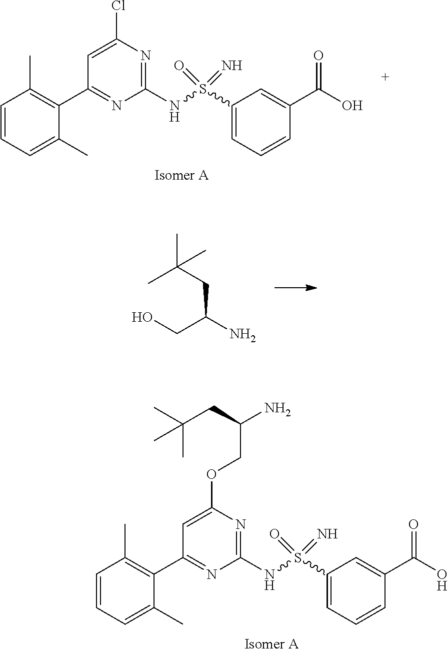

3-[[[4-Chloro-6-(2,6-dimethylphenyl)pyrimidin-2-yl]amino]sulfonimidoyl]benzoic acid isomer A (4 g, 8.5012 mmol) was dissolved in 2-MeTHF (36 mL) and DMF (4 mL). The reaction mixture was cooled to 0° C. and sodium tert-butoxide (4.8 g, 49.946 mmol) was added followed by (2R)-2-amino-4,4-dimethyl-pentan-1-ol (hydrochloride salt) (1.8 g, 10.735 mmol). The reaction was then warmed to room temperature and stirred for 5.5 h. More sodium tert-butoxide (817 mg, 8.5013 mmol) was added and the reaction mixture was stirred 15 min. at room temperature. The reaction was cooled to 0° C. and quenched by adding an aqueous solution of hydrochloric acid (2M, 60 mL). The reaction mixture stood still overnight at room temperature and then the mixture was evaporated to dryness and the residue was purified twice by reverse phase chromatography on a 120 g C₁₈Aq cartridge using a gradient of 10-100% MeCN in acidic water (0.1% HCl) to afford after lyophilization 3-[[[4-[(2R)-2-amino-4,4-dimethyl-pentoxy]-6-(2,6-dimethylphenyl)pyrimidin-2-yl]amino]sulfonimidoyl]benzoic acid (hydrochloride salt) isomer A (2.24 g, 46%) as a pale beige solid. ¹H NMR (400 MHz, DMSO-d6) δ 8.62-8.31 (m, 5H), 8.30-8.21 (m, 2H), 7.83 (t, J=7.8 Hz, 1H), 7.38-7.30 (m, 1H), 7.19 (d, J 7.6 Hz, 2H), 6.55 (br. s., 1H), 4.48 (d, J 12.0 Hz, 1H), 3.80 (dd, J 11.6, 7.5 Hz, 1H), 3.53 (br. s., 1H), 2.08 (br. s, 6H), 1.52 (d, J 5.6 Hz, 2H), 0.92 (s, 9H). ESI-MS m/z calc. 511.2253, found 512.2 (M+1)⁺; Retention time: 2.11 minutes; LC method J.

### Example 14: Preparation of 3-[[[4-[(2R)-2-amino-4,4-dimethyl-pentoxy]-6-(2,6-dimethylphenyl)pyrimidin-2-yl]amino]sulfonimidoyl]benzoic acid isomer B

### Step 1: 3-[[[4-Chloro-6-(2,6-dimethylphenyl)pyrimidin-2-yl]amino]sulfonimidoyl]benzoic acid isomer B

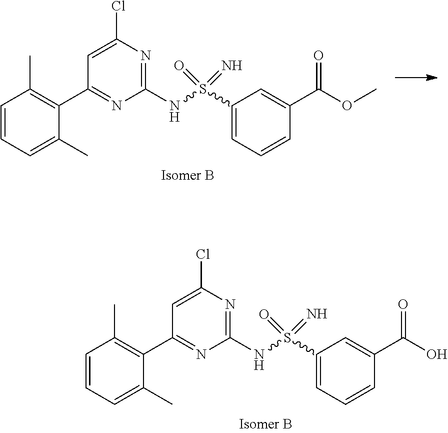

To a solution of methyl 3-[[[4-chloro-6-(2,6-dimethylphenyl)pyrimidin-2-yl]amino]sulfonimidoyl]benzoate isomer B (8.52 g, 17.637 mmol) in tetrahydrofuran (170 mL) and water (170 mL) at 0° C. was added lithium hydroxide hydrate (1.85 g, 44.086 mmol). The resulting light-yellow solution was stirred at room temperature for 16 h. The reaction mixture was diluted with aqueous saturated NH₄Cl (200 mL) and some HCl 1 N (approx. 30 mL) to reach pH=4. The product was extracted with EtOAc (3×200 mL) and the combined organic phases were washed with brine (200 mL), dried with magnesium sulfate, filtered and concentrated to dryness to afford 3-[[[4-chloro-6-(2,6-dimethylphenyl)pyrimidin-2-yl]amino]sulfonimidoyl]benzoic acid isomer B (7.62 g, 96%) as a beige solid. ¹H NMR (400 MHz, DMSO-d6) δ 8.42 (br. s., 1H), 8.10 (d, J 6.8 Hz, 1H), 7.95 (d, J 7.8 Hz, 1H), 7.74 (br. s., 2H), 7.55 (t, J 7.7 Hz, 1H), 7.20-7.13 (m, 1H), 7.03 (d, J 7.3 Hz, 2H), 6.89 (s, 1H), 1.95-1.56 (m, 6H). ESI-MS m z calc. 416.07098, found 417.1 (M+1)⁺; Retention time: 3.58 minutes; LC method J.

### Step 2: 3-[[[4-[(2R)-2-Amino-4,4-dimethyl-pentoxy]-6-(2,6-dimethylphenyl)pyrimidin-2-yl]amino]sulfonimidoyl]benzoic acid isomer B

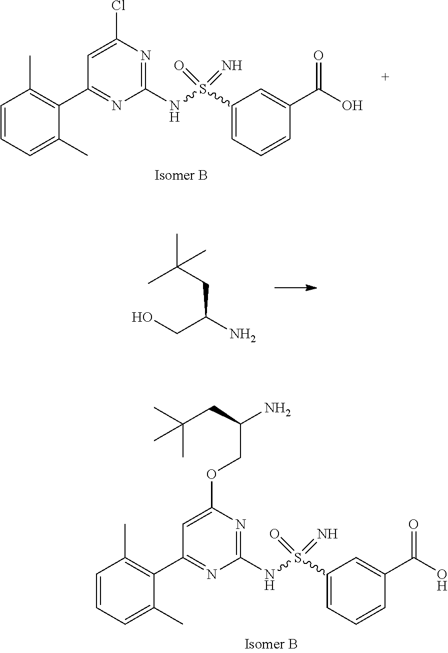

3-[[[4-Chloro-6-(2,6-dimethylphenyl)pyrimidin-2-yl]amino]sulfonimidoyl]benzoic acid isomer B (3.97 g, 8.4279 mmol) was dissolved in 2-MeTHF (36 mL) and DMF (4 mL). The reaction mixture was cooled to 0° C. and sodium tert-butoxide (4.05 g, 42.142 mmol) was added followed by (2R)-2-amino-4,4-dimethyl-pentan-1-ol (hydrochloride salt) (1.7 g, 10.139 mmol). The reaction was then warmed to room temperature and stirred for 6 h. More sodium tert-butoxide (2 g, 20.811 mmol) was added and the mixture was stirred 18 h at room temperature. The reaction was cooled to 0° C. and quenched by adding an aqueous solution of hydrochloric acid (2M, 70 mL). The mixture was evaporated to dryness and the residue was purified twice by reverse phase chromatography on a 120 g C₁₈Aq cartridge using a gradient of 5-100% MeCN in acidic water (0.1% HCl) to afford after lyophilization 3-[[[4-[(2R)-2-amino-4,4-dimethyl-pentoxy]-6-(2,6-dimethylphenyl)pyrimidin-2-yl]amino]sulfonimidoyl]benzoic acid (hydrochloride salt) isomer B (2.13 g, 45%) as an off-white solid. ¹H NMR (400 MHz, DMSO-d6) δ 8.55-8.33 (m, 5H), 8.29-8.20 (m, 2H), 7.82 (t, J=7.8 Hz, 1H), 7.36-7.29 (m, 1H), 7.18 (d, J 7.6 Hz, 2H), 6.53 (br. s., 1H), 4.32 (dd, J 11.7, 7.6 Hz, 1H), 3.96 (d, J 11.5 Hz, 1H), 3.49 (br. s., 1H), 2.07 (br. s., 6H), 1.58-1.47 (m, 2H), 0.93 (s, 9H). ESI-MS m/z calc. 511.2253, found 512.3 (M+1)⁺; Retention time: 2.13 minutes; LC method J.

### Example 15: Preparation of 3-[[4-[(2R)-2-amino-3-(1-methylcyclobutyl)propoxy]-6-(2,6-dimethylphenyl)pyrimidin-2-yl]sulfamoyl]benzoic acid

### Step 1: N-Methoxy-N,1-dimethyl-cyclobutanecarboxamide

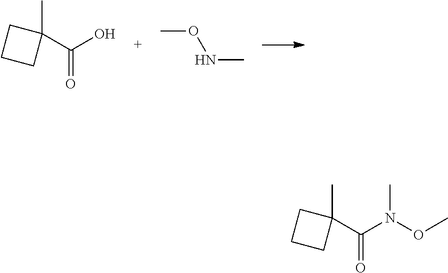

To a solution of 1-methylcyclobutanecarboxylic acid (18 g, 157.70 mmol) in DMF (200 mL) at 0° C. was added N-methoxymethanamine hydrochloride (31 g, 317.81 mmol) followed by HATU (70 g, 184.10 mmol) and triethylamine (50.820 g, 70 mL, 502.22 mmol). The mixture was stirred at 0° C. for 30 minutes and then at room temperature for 18 hours. Water (400 mL) and EtOAc (400 mL) were added, and the mixture was extracted with EtOAc (3×200 mL), washed with an aqueous 1 N HCl solution (2×400 mL), an aqueous saturated solution of sodium bicarbonate (2×400 mL), water (2×400 mL) and brine (2×400 mL), dried over sodium sulfate, filtered and concentrated in vacuo to afford N-methoxy-N,1-dimethyl-cyclobutanecarboxamide (20.5 g, 75%) as a yellow oil. ESI-MS m/z calc. 157.11028, found 158.4 (M+1)⁺; Retention time: 1.49 minutes; LC method I.

### Step 2: 1-Methylcyclobutanecarbaldehyde

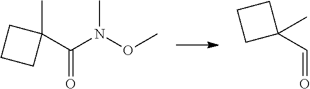

A solution of N-methoxy-N,1-dimethyl-cyclobutanecarboxamide (20 g, 115.01 mmol) in dry dioxane (100 mL) was added to a suspension of LAH (6.5 g, 171.26 mmol) in dry dioxane (200 mL) at 0° C. The mixture was stirred at 0° C. for 5 minutes and then at room temperature for 2 hours. The mixture was then cooled down to 0° C. and water (6.5 mL) was added followed by an aqueous solution of NaOH (15%, 6.5 mL) and then water (19.5 mL). The mixture was stirred at room temperature for 30 minutes and magnesium sulfate was added (10 g). The mixture was filtered on Celite and the filter cake was rinsed with dioxane (100 mL) to afford 1-methylcyclobutanecarbaldehyde (11.28 g, 100%) in a dioxane solution. This solution will be used directly as is in the next reaction as a dioxane solution.

### Step 3: Methyl 2-(tert-butoxycarbonylamino)-3-(1-methylcyclobutyl)prop-2-enoate

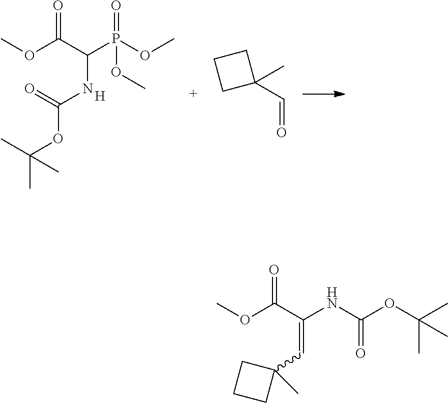

To a stirred solution of 1-methylcyclobutanecarbaldehyde (in a dioxane solution) (11.28 g, 114.93 mmol) at 0° C. was added methyl 2-(tert-butoxycarbonylamino)-2-dimethoxyphosphoryl-acetate (11.5 g, 38.689 mmol) followed by 1,1,3,3-tetramethylguanidine (13.311 g, 14.5 mL, 115.57 mmol). The reaction mixture was stirred at 0° C. for 1 hour and then at room temperature for 24 hours. Water (100 mL) and EtOAc (250 mL) were added, and the mixture was extracted with EtOAc (3×250 mL). The combined organic layers were washed with brine (250 mL), dried over sodium sulfate, filtered and concentrated in vacuo. The crude mixture was purified by flash-chromatography on a silica gel cartridge (120 g Gold), using a gradient of 0 to 40% of EtOAc in heptanes to afford after evaporation methyl 2-(tert-butoxycarbonylamino)-3-(1-methylcyclobutyl)prop-2-enoate (8.6 g, 82%) as a white solid. ESI-MS m/z calc. 269.1627, found 214.2 (M-55)+; Retention time: 1.84 minutes, LC method I.

### Step 4: Methyl (2R)-2-(tert-butoxycarbonylamino)-3-(I-methylcyclobutyl)propanoate

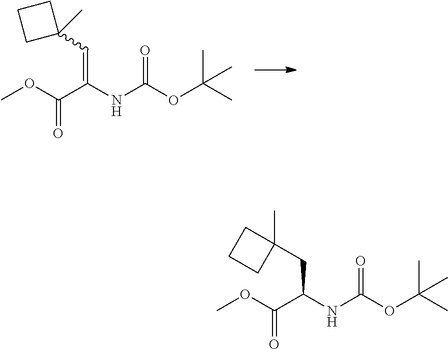 [SMILES: COC(=O)C(=CC1(C)CCC1)NC(=O)OC(C)(C)C ; COC(=O)[C@@H](CC1(C)CCC1)NC(=O)OC(C)(C)C]

Methyl 2-(tert-butoxycarbonylamino)-3-(1-methylcyclobutyl)prop-2-enoate (18 g, 65.962 mmol) was dissolved in ethanol (180 mL) and dioxane (90 mL). Nitrogen was passed through for 15 minutes and then 1,2-bis[(2R,5R)-2,5-diethylphospholano]benzene(1,5-cyclooctadiene)rhodium(I) trifluoromethanesulfonate (2.5 g, 3.4596 mmol) was added. Nitrogen was passed through for 5 minutes and then the mixture was hydrogenated under 65 psi hydrogen pressure and at room temperature for 4 hours. The mixture was concentrated in vacuo to dryness and a solution of EtOAc and heptanes (1:1, 200 mL) was then added to the mixture. The crude solution was filtered on a silica pad and the pad was rinsed with a solution of EtOAc and heptanes (1:1, 400 mL) to afford after evaporation methyl (2R)-2-(tert-butoxycarbonylamino)-3-(1-methylcyclobutyl)propanoate (17.5 g, 93%) as a yellow oil. ¹H NMR (400 MHz, CDCl₃) δ 4.91-4.77 (m, 1H), 4.39-4.24 (m, 1H), 3.72 (s, 3H), 2.01-1.76 (m, 5H), 1.75-1.64 (m, 3H), 1.45 (s, 9H), 1.23 (s, 3H). ESI-MS m/z calc. 271.1784, found 294.2 (M+23)⁺; Retention time: 1.9 minutes; LC method I.

### Step 5: tert-Butyl N-[(1R)-1-(hydroxymethyl)-2-(I-methylcyclobutyl)ethyl]carbamate

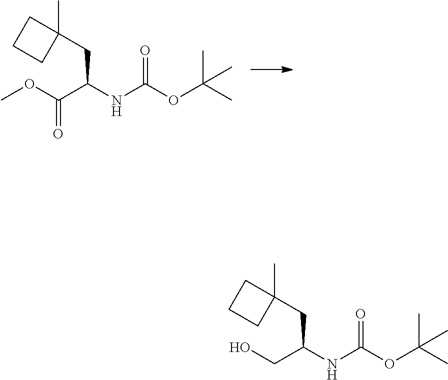 [SMILES: COC(=O)[C@@H](CC1(C)CCC1)NC(=O)OC(C)(C)C ; CC1(C[C@H](CO)NC(=O)OC(C)(C)C)CCC1]

A solution of methyl (2R)-2-(tert-butoxycarbonylamino)-3-(1-methylcyclobutyl)propanoate (17.5 g, 61.267 mmol) in THE (40 mL) at 0° C. was added to a suspension of LAH (3.5 g, 92.216 mmol) in THF (160 mL). The mixture was stirred at 0° C. for 15 minutes and then at room temperature for 2 hours. The mixture was then cooled down to 0° C. and water (3.5 mL) was added followed by an aqueous solution of NaOH (15%, 3.5 mL) and then by water (10.5 mL). The mixture was stirred at room temperature for 30 minutes and then magnesium sulfate (2 g) was added. The mixture was filtered on Celite and the filter cake was washed with EtOAc (100 mL). The filtrate was then concentrated in vacuo to afford the crude tert-butyl N-[(1R)-1-(hydroxymethyl)-2-(1-methylcyclobutyl)ethyl]carbamate (15.3 g, 97%) as a colorless oil. ¹H NMR (400 MHz, CDCl₃) δ 4.51 (br. s, 1H), 3.72 (br. s, 1H), 3.65-3.56 (m, 1H), 3.51-3.41 (m, 1H), 2.47 (br. s, 1H), 2.00-1.90 (m, 1H), 1.89-1.76 (m, 3H), 1.74-1.65 (m, 2H), 1.61-1.49 (m, 2H), 1.44 (s, 9H), 1.19 (s, 3H). ESI-MS m/z calc. 243.1834, found 188.2 (M-55)⁺; Retention time: 1.74 minutes; LC method I.

### Step 6: (2R)-2-Amino-3-(I-methylcyclobutyl)propan-1-ol

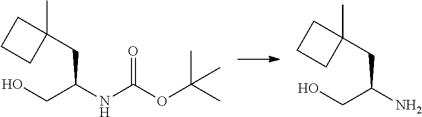

To a solution of tert-butyl N-[(1R)-1-(hydroxymethyl)-2-(1-methylcyclobutyl)ethyl]carbamate (15.3 g, 59.731 mmol) in dry DCM (150 mL) was added HCl (in dioxane) (150 mL of 4 M, 600.00 mmol) at room temperature. After 18 hours, the solvent was removed in vacuo to afford after co-evaporation with MeCN (2×100 mL) (2R)-2-amino-3-(1-methylcyclobutyl) propan-1-ol (hydrochloride salt) (11 g, 97%) as a white solid. ¹H NMR (400 MHz, DMSO-d6) δ 7.93 (br. s, 3H), 5.33 (br. s, 1H), 3.56 (dd, J 11.5, 3.4 Hz, 1H), 3.39-3.31 (m, 1H, overlapped with water), 3.04 (br. s, 1H), 1.95-1.79 (m, 3H), 1.78-1.56 (m, 5H), 1.12 (s, 3H). Several batches from 3 different reactions of (2R)-2-amino-3-(1-methylcyclobutyl)propan-1-ol (hydrochloride salt) (1.25 g, 6.6087 mmol, 1.28 g, 6.7673 mmol, and 11 g, 58.156 mmol) were combined in water (75 mL). The resulting mixture was then lyophilized to afford (2R)-2-amino-3-(1-methylcyclobutyl)propan-1-ol (hydrochloride salt) (12.8 g, 95%) as a white solid. ¹H NMR (400 MHz, DMSO-d6) δ 7.97 (br. s, 3H), 5.33 (br. s, 1H), 3.60-3.52 (m, 1H), 3.39-3.30 (m, 1H, overlapped with water), 3.04 (br. s, 1H), 1.94-1.79 (m, 3H), 1.78-1.57 (m, 5H), 1.12 (s, 3H). ESI-MS m/z calc. 143.13101, found 144.4 (M+1)⁺; Retention time: 0.56 minutes; LC method I.

### Step 7: Benzyl N-[(1R)-1-(hydroxymethyl)-2-(I-methylcyclobutyl)ethyl]carbamate

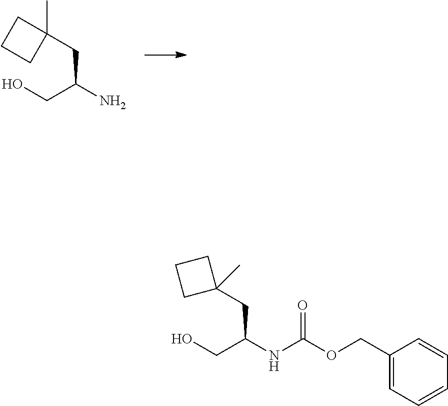

To a stirred suspension of (2R)-2-amino-3-(1-methylcyclobutyl)propan-1-ol (12.3 g, 81.584 mmol) in dry THE (250 mL) at 0° C. were added triethylamine (25.410 g, 35 mL, 251.11 mmol) followed by N-(benzyloxycarbonyloxy)succinimide (24.5 g, 98.307 mmol). The reaction was stirred at 0° C. for 15 minutes and then at room temperature for 4 hours. Water (250 mL) and EtOAc (250 mL) were added, and the mixture was extracted with EtOAc (3×250 mL). The combined organic layers were washed with water (3×250 mL) and brine (250 mL), dried over sodium sulfate, filtered, and concentrated in vacuo. The crude mixture was purified twice by flash-chromatography on a 330 g silica gel cartridge, eluting with a gradient of 0 to 100% of EtOAc in heptanes and then by reverse phase chromatography on a 275 g C₁₈ GOLD cartridge, eluting with a gradient of 40 to 100% of MeOH in acidic water (0.1% v/v of formic acid in water). The fractions containing the desired product were combined and the organic solvent was evaporated. EtOAc (500 mL) was then added, and the mixture was extracted with EtOAc (3×500 mL). The combined organic layers were washed with brine (1×500 mL), dried over sodium sulfate, filtered, and concentrated in vacuo. The product was then separated by SFC (Column Lux 5 m, Cellulose 4, 250×21.2 mm, 21.5 mg/injection, concentration 53.8 mg/mL, Injected volume 400 μL, Column T=40° C., Flow rate 75 mL/min, 20% MeOH). The fractions containing the desired product were combined and the solvent was evaporated to afford benzyl N-[(1R)-1-(hydroxymethyl)-2-(1-methylcyclobutyl)ethyl]carbamate (13.5 g, 58%) as a yellow oil. ¹H NMR (400 MHz, CDCl₃) δ 7.40-7.29 (m, 5H), 5.10 (s, 2H), 4.81 (br. s, 1H), 3.87-3.75 (m, 1H), 3.70-3.60 (m, 1H), 3.54-3.46 (m, 1H), 2.23 (br. s, 1H), 2.01-1.90 (m, 1H), 1.90-1.75 (m, 3H), 1.74-1.65 (m, 2H), 1.64-1.52 (m, 2H), 1.19 (s, 3H). ESI-MS m/z calc. 277.1678, found 278.2 (M+1)⁺; Retention time: 1.74 minutes; LC method I. The fractions containing the other enantiomer were combined and the solvent was concentrated in vacuo. The product was purified twice by reverse phase chromatography on a 80 g C₁₈ GOLD cartridge, eluting with a gradient of MeOH to 50% of 100 in acidic water (0.1% v/v of formic acid in water) and then on a 80 g Cis GOLD cartridge, eluting with a gradient of MeCN to 50% of 100 in acidic water (0.1% v/v of formic acid in water). The fractions containing the desired product were combined and the organic solvent was evaporated. EtOAc (50 mL) was then added, and the mixture was extracted with EtOAc (3×50 mL). The combined organic layers were washed with brine (1×50 mL), dried over sodium sulfate, filtered and concentrated in vacuo to afford benzyl N-[(1S)-1-(hydroxymethyl)-2-(1-methylcyclobutyl)ethyl]carbamate (525 mg, 2%) as a yellow oil. ¹H NMR (400 MHz, CDCl₃) δ 7.38-7.31 (m, 5H), 5.11 (s, 2H), 4.80 (br. s, 1H), 3.86-3.76 (m, 1H), 3.71-3.62 (m, 1H), 3.55-3.47 (m, 1H), 2.00-1.90 (m, 1H), 1.88-1.76 (m, 3H), 1.74-1.65 (m, 2H), 1.64-1.52 (m, 2H), 1.19 (s, 3H), 1 missing proton (labile proton). ESI-MS m/z calc. 277.1678, found 278.2 (M+1)⁺; Retention time: 1.75 minutes; LC method I.

### Step 8: (2R)-2-Amino-3-(I-methylcyclobutyl)propan-1-ol

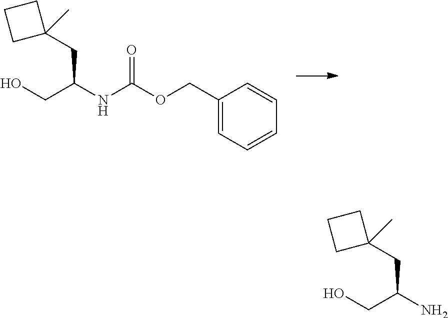

To a degassed solution of benzyl N-[(1R)-1-(hydroxymethyl)-2-(1-methylcyclobutyl)ethyl]carbamate (13.5 g, 47.165 mmol) in methanol (250 mL) was added 10% Palladium on carbon 50% wet (5.2 g, 2.4431 mmol). After purging with nitrogen for 5 minutes, hydrogen was bubbled into the solution for 5 minutes after which the mixture was stirred at room temperature under a hydrogen atmosphere (1 atm.) for 6 hours. The mixture was filtered through a pad of Celite® and the pad was rinsed with methanol (100 mL). The filtrate was concentrated in vacuo and then acidified by adding hydrogen chloride solution (in methanol) (50 mL of 3 M, 150.00 mmol) to the product. The mixture was stirred 5 minutes at room temperature and then concentrated in vacuo to afford after lyophilization (2R)-2-amino-3-(1-methylcyclobutyl)propan-1-ol (hydrochloride salt) (7.68 g, 86%) as a white solid. ¹H NMR (400 MHz, DMSO-d6) δ 7.28 (br. s, 3H), 5.23 (br. s, 1H), 3.57-3.47 (m, 1H), 3.34-3.25 (m, 1H), 3.05-2.95 (m, 1H), 1.95-1.70 (m, 4H), 1.70-1.58 (m, 3H), 1.58-1.50 (m, 1H), 1.11 (s, 3H). ESI-MS m/z calc. ESI-MS m/z calc. 143.13101, found 144.4 (M+1)⁺; Retention time: 0.64 minutes; LC method I.

### Step 9: 3-[[4-[(2R)-2-Amino-3-(1-methylcyclobutyl)propoxy]-6-(2,6-dimethylphenyl)pyrimidin-2-yl]sulfamoyl]benzoic acid

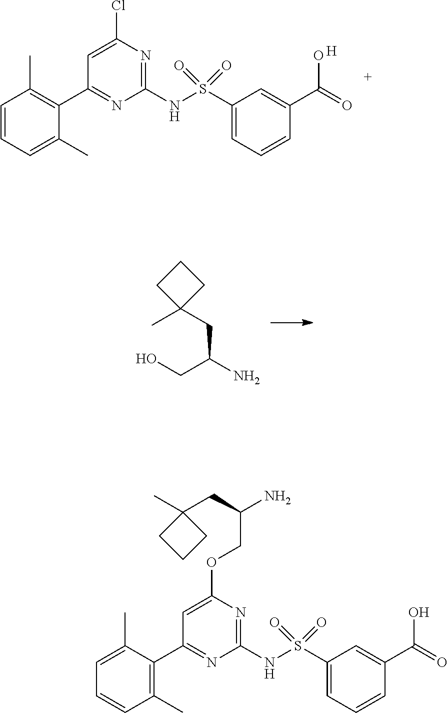

A solution of (2R)-2-amino-3-(1-methylcyclobutyl)propan-1-ol (7.65 g, 50.741 mmol) in anhydrous N,N-dimethylformamide (40 mL) was added to a solution of 3-[[4-chloro-6-(2,6-dimethylphenyl)pyrimidin-2-yl]sulfamoyl]benzoic acid (23 g, 55.042 mmol) in 2-methyltetrahydrofuran (200 mL). The mixture was cooled down to 10-15° C. and then sodium tert-butoxide (30 g, 312.16 mmol) was added. The reaction was stirred at 10-15° C. for 2 hours, then cooled down to 0° C. and quenched by the addition of an aqueous solution of 1 N HCl (300 mL). The biphasic mixture was stirred for 30 minutes. The layers were then separated, and the aqueous layer was extracted with 2-methyltetrahydrofuran (5×500 mL). The combined organic layers were washed with water (3×500 mL) and brine (1×500 mL), dried over magnesium sulfate, filtered and concentrated in vacuo. The crude mixture was purified by reverse phase chromatography on a 275 g C₁₈ GOLD cartridge, eluting with a gradient of 20 to 100% of MeOH in acidic water (0.1% of hydrochloric acid in water) to afford after evaporation 3-[[4-[(2R)-2-amino-3-(1-methylcyclobutyl)propoxy]-6-(2,6-dimethylphenyl)pyrimidin-2-yl]sulfamoyl]benzoic acid (hydrochloride salt) (24.25 g, 78%) as a white solid. ¹H NMR (400 MHz, DMSO-d6) δ 8.48-8.35 (m, 4H), 8.13 (t, J 9.3 Hz, 2H), 7.71 (t, J 7.7 Hz, 1H), 7.25 (t, J 7.6 Hz, 1H), 7.12 (d, J 7.6 Hz, 2H), 6.33 (s, 1H), 4.32 (dd, J 11.6, 2.6 Hz, 1H), 4.06 (dd, J 11.7, 6.1 Hz, 1H), 3.47 (br. s, 1H), 2.00 (s, 6H), 1.93-1.83 (m, 2H), 1.82-1.62 (m, 5H), 1.58-1.47 (m, 1H), 1.16 (s, 3H). 2H missing, labile protons. ESI-MS m/z calc. 524.20935, found 525.3 (M+1)⁺; Retention time: 2.49 minutes; LC method J.

### Example 16: Preparation of 3-[[4-(2-amino-5-fluoro-5-methyl-hexoxy)-6-(2,6-dimethylphenyl)pyrimidin-2-yl]sulfamoyl]benzoic acid

### Step 1: Ethyl 3-hydroxy-3-methyl-butanoate

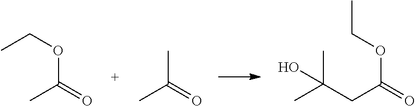

Ethyl acetate (5.0512 g, 5.6 mL, 57.332 mmol) was added dropwise to a solution of (bis(trimethylsilyl)amino)lithium (in THF) (39 mL of 1.5 M, 58.500 mmol) in THE (56 mL) at −78° C. The reaction mixture was stirred at this temperature for 30 min. Acetone (3.9550 g, 5 mL, 68.097 mmol) was added and the reaction mixture was left stirring 10 min. HCl (2 M, 35 ml) was added to the reaction mixture which was then left to warm up to room temperature. The reaction mixture was extracted with ethyl acetate (2×100 mL). The combined organic phases were washed with saturated aqueous sodium bicarbonate (50 mL), dried with sodium sulfate, filtered and concentrated under reduced pressure to afford ethyl 3-hydroxy-3-methyl-butanoate (7.84 g, 89%) as a clear yellow oil ¹H NMR (400 MHz, CDCl₃) δ 4.18 (q, J 7.1 Hz, 2H), 3.59 (s, 1H), 2.48 (s, 2H), 1.31-1.26 (m, 9H). ESI-MS m/z calc. 146.0943, found 169.2 (M+23)+; Retention time: 1.3 minutes; LC method I.

### Step 2: Ethyl 3-fluoro-3-methyl-butanoate

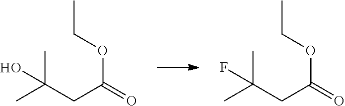

Deoxo-Fluor (solution in toluene) (26 g, 50% w/w, 58.759 mmol) was added to a solution of ethyl 3-hydroxy-3-methyl-butanoate (7.5 g, 48.740 mmol) in DCM (125 mL) at −78° C. The reaction was then left to warm-up to room temperature and stirred for 4 h. The reaction mixture was quenched with aqueous sodium bicarbonate (200 mL). The aqueous phase was extracted with DCM (2×100 mL) and the combined organic phases were washed with saturated aqueous ammonium chloride (100 mL), dried with magnesium sulfate, filtered and concentrated under reduced pressure to provide crude ethyl 3-fluoro-3-methyl-butanoate (4.8 g, 53%) as a clear oil ¹H NMR (400 MHz, CDCl₃) δ 4.16 (q, J 7.2 Hz, 2H), 2.66 (d, J 16.1 Hz, 2H), 1.49 (d, J 21.8 Hz, 6H), 1.28 (t, J 7.2 Hz, 3H). Which was used directly in the next step without further purification.

### Step 3: 3-Fluoro-3-methyl-butanal

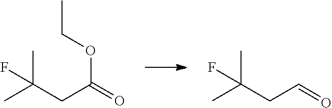

DIBAL (in toluene) (8.7 mL of 1 M, 8.7000 mmol) was slowly added to a solution of ethyl 3-fluoro-3-methyl-butanoate (1 g, 5.3990 mmol) in DCM (10 mL) at −78° C. The reaction mixture was left to stir at this temperature for 1 h. The reaction mixture was quenched with concentrated aqueous ammonium chloride (20 mL) and 1 N HCl (5 mL). The reaction mixture was left to warm-up to room temperature and left stirring for 30 min. DCM (100 mL) was added to the mixture and shaken. The aqueous phase was separated and washed with more DCM (25 mL). The combined organic phases were dried with sodium sulfate overnight then filtered to provide a ˜0.3% solution of 3-fluoro-3-methyl-butanal (185 g, 99%) as a clear solution. This solution was used directly in the next step.

### Step 4: Methyl 2-(tert-butoxycarbonylamino)-5-fluoro-5-methyl-hex-2-enoate

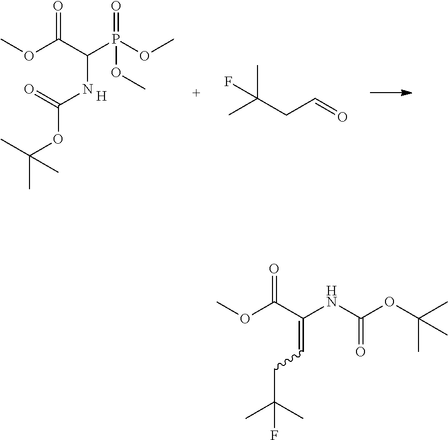

To a stirred solution of 3-fluoro-3-methyl-butanal (3% in DCM) (185 g, 5.3302 mmol) at 0° C. was added methyl 2-(tert-butoxycarbonylamino)-2-dimethoxyphosphoryl-acetate (500 mg, 1.6821 mmol) followed by 1,1,3,3-tetramethylguanidine (580 mg, 5.0357 mmol). The reaction mixture was stirred at 0° C. for 1 hour and then at room temperature for 18 hours. Water (100 mL) and DCM (100 mL) were added and the mixture was extracted with DCM (3×100 mL). The combined organic layers were washed with brine (50 mL), dried over sodium sulfate, filtered and concentrated under reduced pressure. The resulting residue was purified by flash-chromatography on a 40 g silica gel cartridge, using a gradient of 0 to 40% of EtOAc in heptanes to provide methyl 2-(tert-butoxycarbonylamino)-5-fluoro-5-methyl-hex-2-enoate (383 mg, 83%) as a white solid ¹H NMR (400 MHz, CDCl₃) δ 6.62 (t, J 7.3 Hz, 1H), 6.11 (br. s., 1H), 3.80 (s, 3H), 2.54 (dd, J 20.3, 7.6 Hz, 2H), 1.47 (s, 9H), 1.40 (d, J 21.5 Hz, 6H). 19F NMR (377 MHz, CDCl₃) δ −138.20 (br. s., 1F). ESI-MS m/z calc. 275.1533, found 298.2 (M+23)+; Retention time: 1.73 minutes; LC method I.

### Step 5: Methyl 2-(tert-butoxycarbonylamino)-5-fluoro-5-methyl-hexanoate

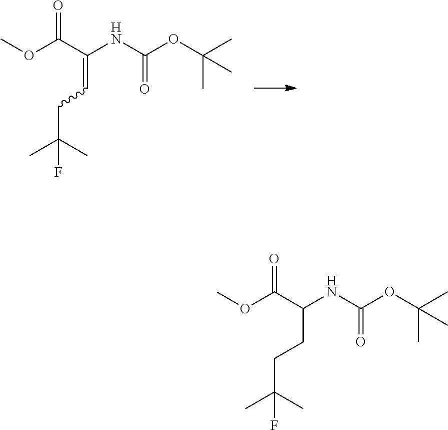 [SMILES: COC(=O)C(=CCC(C)(C)F)NC(=O)OC(C)(C)C ; COC(=O)C(CCC(C)(C)F)NC(=O)OC(C)(C)C]

Palladium (on carbon) (400 mg, 0.1879 mmol) was added to a solution of methyl 2-(tert-butoxycarbonylamino)-5-fluoro-5-methyl-hex-2-enoate (380 mg, 1.3802 mmol) in Methanol (4 mL) and hydrogen was injected into the suspension with a hydrogen balloon equipped with a thin needle on a continuous manner for 30 min. The crude mixture was filtered with a syringe filter and concentrated under reduced pressure. The resulting residue was purified by reverse phase chromatography on a C₁₈ column using 5 to 100% acetonitrile in acid water (with 0.1% formic acid) to provide methyl 2-(tert-butoxycarbonylamino)-5-fluoro-5-methyl-hexanoate (250 mg, 65%) as a clear oil ¹H NMR (400 MHz, CDCl₃) δ 5.03 (d, J 7.1 Hz, 1H), 4.38-4.26 (m, 1H), 3.76 (s, 3H), 2.04-1.89 (m, 1H), 1.80-1.71 (m, 1H), 1.69-1.57 (m, 2H), 1.45 (s, 9H), 1.34 (d, J=21.5 Hz, 6H). ESI-MS m/z calc. 277.1689, found 300.2 (M+23)+; Retention time: 1.76 minutes; LC method I.

### Step 6: tert-Butyl N-[4-fluoro-1-(hydroxymethyl)-4-methyl-pentyl]carbamate

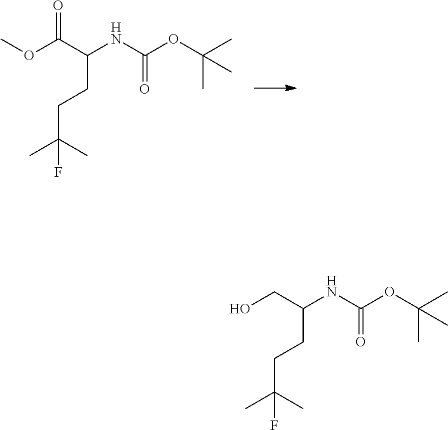 [SMILES: COC(=O)C(CCC(C)(C)F)NC(=O)OC(C)(C)C ; CC(C)(F)CCC(CO)NC(=O)OC(C)(C)C]

To a solution of methyl 2-(tert-butoxycarbonylamino)-5-fluoro-5-methyl-hexanoate (230 mg, 0.8293 mmol) in ethanol (6 mL) at 0° C. was added lithium borohydride (340 mg, 15.608 mmol). This reaction mixture was stirred 1 h at this temperature and then left to warm-up to room temperature stirred for another 30 min, then water (20 mL) was added to the reaction mixture which was then left stirring overnight at room temperature. The reaction mixture was transferred into a cold aqueous 0.1N HCl (40 mL) solution and this mixture was extracted with DCM (3×50 mL). The combined organic phases were dried with sodium sulfate, filtered and concentrated under reduced pressure to provide tert-butyl N-[4-fluoro-1-(hydroxymethyl)-4-methyl-pentyl]carbamate (240 mg, 99%) as a clear oil ¹H NMR (400 MHz, CDCl₃) δ 4.66 (br. s., 1H), 3.78-3.53 (m, 3H), 2.32 (br. s., 1H), 1.82-1.58 (m, 4H), 1.46 (s, 9H), 1.36 (d, J 21.8 Hz, 6H). 19F NMR (377 MHz, CDCl₃) δ −139.16 (br. s., 1F). ESI-MS m/z calc. 249.174, found 272.2 (M+23)+; Retention time: 1.63 minutes; LC method I.

### Step 7: 2-Amino-5-fluoro-5-methyl-hexan-1-ol

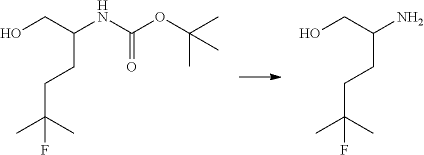

Hydrogen chloride (in dioxane) (2 mL of 4 M, 8.0000 mmol) was added to a solution of tert-butyl N-[4-fluoro-1-(hydroxymethyl)-4-methyl-pentyl]carbamate (240 mg, 0.8182 mmol) in DCM (2 mL) and the reaction mixture was left stirring at room temperature for 1 h. The reaction mixture was concentrated under reduced pressure and the resulting residue was dissolved in pure water and concentrated under reduced pressure the resulting residue was then redissolved in water and lyophilized to provide 2-amino-5-fluoro-5-methyl-hexan-1-ol (hydrochloride salt) (138 mg, 86%) white solid ¹H NMR (400 MHz, DMSO-d6) δ 7.95 (br. s., 3H), 5.30 (t, J 5.0 Hz, 1H), 3.59 (dt, J 11.5, 4.4 Hz, 1H), 3.45 (dt, J 11.4, 5.7 Hz, 1H), 3.10-2.99 (m, 1H), 1.71-1.57 (m, 4H), 1.30 (d, J 22.0 Hz, 6H). 19F NMR (377 MHz, DMSO-d6) δ −136.12 (nonu, J 20.4 Hz, 1F). ESI-MS m/z calc. 149.1216, found 150.2 (M+1)⁺; Retention time: 0.29 minutes; LC method I.

### Step 8: 3-[[4-(2-Amino-5-fluoro-5-methyl-hexoxy)-6-(2,6-dimethylphenyl)pyrimidin-2-yl]sulfamoyl]benzoic acid

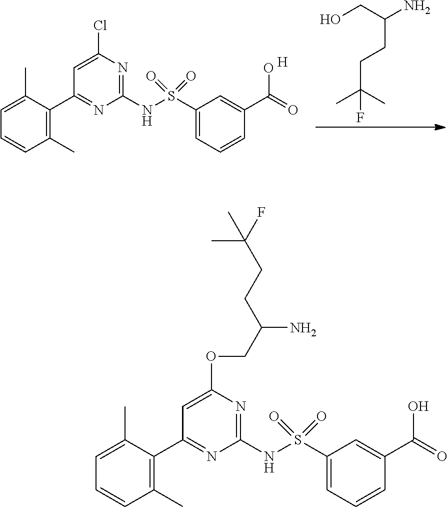

A flame-dried flask under nitrogen atmosphere was charged with 3-[[4-chloro-6-(2,6-dimethylphenyl)pyrimidin-2-yl]sulfamoyl]benzoic acid (325 mg, 0.7778 mmol), 2-amino-5-fluoro-5-methyl-hexan-1-ol (hydrochloride salt) (134 mg, 0.6856 mmol), 2-MeTHF (15 mL) and anhydrous DMF (1.5 mL). The reaction mixture was cooled down to 0° C. then sodium tert-butoxide (375 mg, 3.9020 mmol) was added. The reaction was stirred for 5 minutes at 0° C. then allowed to reach room temperature and stirred at room temperature for 45 minutes. The reaction was then cooled down to 0° C. then diluted with 2-methyltetrahydrofuran (150 mL) and quenched by the addition of an aqueous solution of 1 N hydrochloric acid (150 mL). The layers were separated, and the aqueous layer was extracted with 2-methyltetrahydrofuran (2×150 mL). The combined organic layers were dried over sodium sulfate, filtered and concentrated under reduced pressure and the resulting residue was purified by reverse phase chromatography on a C₁₈ using a 50 g Gold column and eluting with a 5 to 100% gradient of acetonitrile in acidic water (containing 0.1% v/v of hydrochloric acid). The desired fractions were concentrated under reduced pressure, then freeze-dried to afford 3-[[4-(2-amino-5-fluoro-5-methyl-hexoxy)-6-(2,6-dimethylphenyl)pyrimidin-2-yl]sulfamoyl]benzoic acid (hydrochloride salt) (305 mg, 64%) as a white fluffy solid ¹H NMR (400 MHz, DMSO-d6) δ 13.32 (br. s., 1H), 12.60-11.86 (m, 1H), 8.45 (t, J 1.6 Hz, 1H), 8.40-8.03 (m, 5H), 7.69 (t, J 7.7 Hz, 1H), 7.32-7.20 (m, 1H), 7.18-7.06 (m, 2H), 6.32 (br. s., 1H), 4.49-4.34 (m, 1H), 4.28 (dd, J 11.9, 6.2 Hz, 1H), 3.61-3.50 (m, 1H), 2.00 (br. s., 6H), 1.83-1.61 (m, 4H), 1.38-1.27 (m, 6H). 19F NMR (377 MHz, DMSO-d6) δ −136.46 (s, 1F). ESI-MS m/z calc. 530.1999, found 531.1 (M+1)⁺; Retention time: 2.42 minutes; LC method J.

### Example 17: Preparation of 3-[[4-(2-amino-4,4-dimethyl-hexoxy)-6-(2,6-dimethylphenyl)pyrimidin-2-yl]sulfamoyl]benzoic acid

### Step 1: Diethyl 2-(1,1-dimethylpropyl)propanedioate

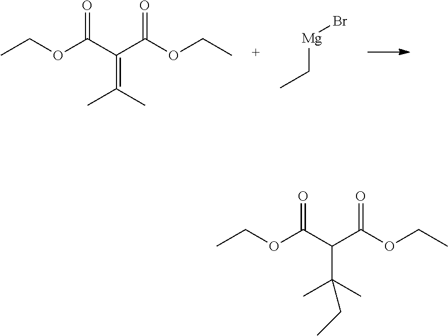

To a solution of diethyl isopropylidenemalonate (2 g, 9.9884 mmol) in THE (60 mL) was added CuI (2.85 g, 14.965 mmol). After stirring for 30 minutes at 0° C., ethylmagnesium bromide solution in THF (30 mL of 1 M, 30.000 mmol) was added dropwise and the mixture was stirred at 0° C. for 3 hours. The mixture was quenched with 1 N HCl (50 mL) and extracted with ethyl acetate (2×60 mL). The combined organic phases was washed with brine (2×50 mL), dried over anhydrous sodium sulfate, filtered and concentrated under reduced pressure to afford diethyl 2-(1,1-dimethylpropyl)propanedioate (2.4 g, 99%) as a brownish oil. ¹H NMR (400 MHz, CDCl₃) δ 4.18 (q, J 7.2 Hz, 4H), 3.33 (s, 1H), 1.48 (q, J 7.4 Hz, 2H), 1.27 (t, J 7.2 Hz, 6H), 1.09 (s, 6H), 0.87 (t, J 7.6 Hz, 3H). ESI-MS m/z calc. 230.15181, found 231.2 (M+1)⁺; Retention time: 1.98 minutes; LC method I.

### Step 2: 3,3-Dimethylpentanoic acid

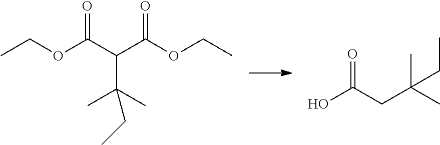

To a solution of diethyl 2-(1,1-dimethylpropyl)propanedioate (2.4 g, 9.9001 mmol) in DMSO (50 mL) and water (10 mL) was added lithium hydroxide hydrate (2.1 g, 50.043 mmol) and the mixture was stirred at 120° C. for 22 hours. The mixture was acidified to pH 2-3 with iN hydrochloric acid and extracted with ethyl acetate (2×40 mL). The combined organic layers were washed with brine (3×30 mL) and water (30 mL), dried over sodium sulfate, filtered, and concentrated under reduced pressure to give 3,3-dimethylpentanoic acid (1.23 g, 91%) as a yellow oil. ¹H NMR (400 MHz, CDCl₃) δ 2.23 (s, 2H), 1.39 (q, J 7.4 Hz, 2H), 1.02 (s, 6H), 0.87 (t, J 7.5 Hz, 3H); one labile proton missing. ESI-MS m/z calc. 130.09938, found 131.2 (M+1)⁺; Retention time: 1.6 minutes; LC method I.

### Step 3: N-Methoxy-N,3,3-trimethyl-pentanamide

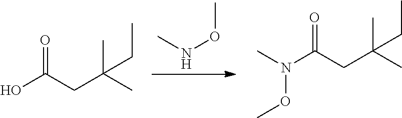

To a solution of 3,3-dimethylpentanoic acid (1.23 g, 8.9757 mmol) in DMF (28 mL) was added at 15-20° C. (water bath)N-methoxymethanamine (hydrochloride salt) (1.1 g, 11.277 mmol), DIPEA (3.6358 g, 4.9 mL, 28.132 mmol) and then HATU (5.39 g, 14.176 mmol). The mixture was stirred for 16 hours at room temperature. The mixture was diluted with water (50 mL) and extracted with MTBE (2×75 mL). The combined organic phases were washed with brine (5×50 mL), dried over anhydrous sodium sulfate, filtered and concentrated under reduced pressure. Purification by silica gel chromatography (Column: 100 g. Gradient: 0-20% ethyl acetate in heptanes) afforded N-methoxy-N,3,3-trimethyl-pentanamide (1.19 g, 76%) as a colorless oil. ¹H NMR (400 MHz, CDCl₃) δ 3.67 (s, 3H), 3.18 (s, 3H), 2.31 (s, 2H), 1.40 (q, J 7.4 Hz, 2H), 1.00 (s, 6H), 0.86 (t, J=7.5 Hz, 3H). ESI-MS m/z calc. 173.14159, found 174.2 (M+1)⁺; Retention time: 1.72 minutes; LC method I.

### Step 4: 3,3-Dimethylpentanal

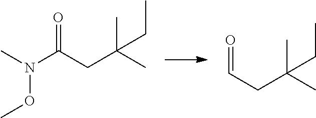

To a solution of N-methoxy-N,3,3-trimethyl-pentanamide (1.12 g, 6.3999 mmol) in THF (20 mL) was added lithium aluminum hydride (736 mg, 19.392 mmol) at 0° C. The mixture was stirred at 0° C. for 1 hour. The reaction mixture was slowly quenched with water (40 mL) and extracted with MTBE (2×30 mL). The combined organic layers were washed with brine (3×45 mL), dried over sodium sulfate and filtered to give crude 3,3-dimethylpentanal (730.77 mg, 100%) as a colorless solution in MTBE/THF.

### Step 5: 2-(Benzylamino)-4,4-dimethyl-hexanenitrile

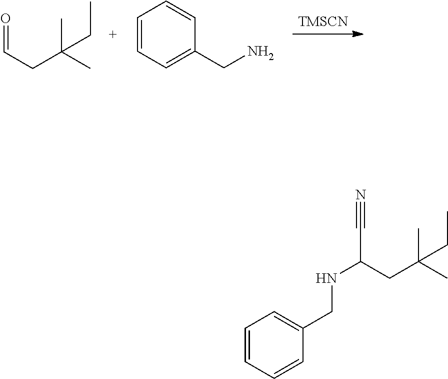

A three-neck round bottom flask equipped with a 6N NaOH trap, was charged with 3,3-dimethylpentanal (730.77 mg, 6.3999 mmol) as a 3:1 MTBE/THF solution (˜80 mL) and benzylamine (765.18 mg, 0.78 mL, 7.1410 mmol). To the mixture was slowly added at 0° C. acetic acid (411.84 mg, 0.39 mL, 6.8581 mmol) and then trimethylsilylcyanide (642.33 mg, 0.81 mL, 6.4747 mmol). The mixture was warmed to room temperature and stirred for 18 hours. The mixture was diluted with water (50 mL) and extracted with ethyl acetate (2×30 mL). The combined organic layers were washed with brine (2×40 mL), dried over anhydrous sodium sulfate, filtered, and concentrated under reduced pressure. Purification by chromatography on silica gel (Column: 120 g. Gradient: 0-10% methanol in dichloromethane) afforded 2-(benzylamino)-4,4-dimethyl-hexanenitrile (761 mg, 49%) as a light yellow oil. ¹H NMR (400 MHz, CDCl₃) δ 7.40-7.28 (m, 5H), 4.08 (d, J 12.7 Hz, 1H), 3.83 (d, J 13.0 Hz, 1H), 3.51 (dd, J 7.6, 5.6 Hz, 1H), 1.83 (dd, J 14.2, 7.6 Hz, 1H), 1.66 (dd, J 14.1, 5.5 Hz, 1H), 1.34-1.25 (m, 3H), 0.94 (s, 6H), 0.84 (t, J 7.6 Hz, 3H). ESI-MS m/z calc. 230.1783, found 231.4 (M+1)⁺; Retention time: 1.95 minutes; LC method I.

### Step 6: 2-(Benzylamino)-4,4-dimethyl-hexanoic acid

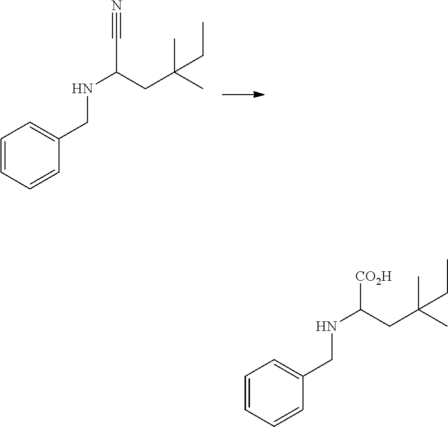 [SMILES: CCC(C)(C)CC(C#N)NCc1ccccc1 ; CCC(C)(C)CC(NCc1ccccc1)C(=O)O]

2-(Benzylamino)-4,4-dimethyl-hexanenitrile (761 mg, 3.1319 mmol) was dissolved in acetic acid (4.2240 g, 4 mL, 70.339 mmol) and hydrochloric acid (24 mL of 12 M, 288.00 mmol) and stirred at 100° C. for 96 hours. The solution was cooled to 10-15° C., the pH was increased to 3-4 with saturated sodium bicarbonate aqueous solution, the mixture was filtered and dried under vacuum to afford 2-(benzylamino)-4,4-dimethyl-hexanoic acid (685 mg, 88%) as a white powder. ¹H NMR (400 MHz, MeOH-d4) δ 7.52-7.42 (m, 5H), 4.15 (d, J 13.2 Hz, 1H), 4.05 (d, J 13.2 Hz, 1H), 3.50 (dd, J 9.3, 3.4 Hz, 1H), 1.94 (dd, J 14.1, 9.4 Hz, 1H), 1.51 (dd, J 13.8, 3.5 Hz, 1H), 1.38-1.29 (m, 2H), 0.93 (s, 6H), 0.84 (t, J 7.5 Hz, 3H); two labile protons missing. ESI-MS m/z calc. 249.17288, found 250.2 (M+1)⁺; Retention time: 1.32 minutes; LC method I.

### Step 7: 2-(Benzylamino)-4,4-dimethyl-hexan-1-ol

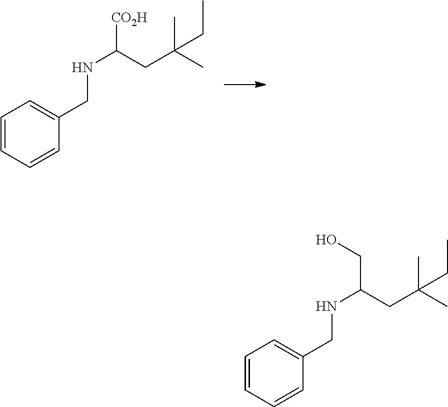 [SMILES: CCC(C)(C)CC(NCc1ccccc1)C(=O)O ; CCC(C)(C)CC(CO)NCc1ccccc1]

To a solution of 2-(benzylamino)-4,4-dimethyl-hexanoic acid (685 mg, 2.7444 mmol) in anhydrous THE (10 mL) was added drop wise at 0° C. borane tetrahydrofuran complex solution in THE (8.3 mL of 1 M, 8.3000 mmol). The reaction was stirred for 30 minutes at 0° C. and then at room temperature for 18 hours. The reaction was cooled down to 0° C. then quenched by the slow addition of methanol (10 mL) and concentrated under reduced pressure. The resulting residue was partitioned between ethyl acetate (50 mL) and 1M sodium hydroxide aqueous solution (40 mL). The biphasic mixture was vigorously stirred until complete solubilization, the layers were separated, and the aqueous layer was extracted ethyl acetate (3×40 mL). The combined organic layers were washed with brine (30 mL), dried over anhydrous sodium sulfate, filtered and concentrated under reduced pressure to afford 2-(benzylamino)-4,4-dimethyl-hexan-1-ol (570 mg, 84%) a colorless oil. ¹H NMR (400 MHz, CDCl₃) δ 7.42-7.28 (m, 5H), 3.82 (d, J 12.7 Hz, 1H), 3.74 (d, J 12.7 Hz, 1H), 3.68 (dd, J 10.5, 3.9 Hz, 1H), 3.25 (dd, J=10.5, 6.4 Hz, 1H), 2.79-2.73 (m, 1H), 1.39-1.34 (m, 2H), 1.30-1.22 (m, 4H), 0.88 (s, 3H), 0.88 (s, 3H), 0.83 (t, J 7.6 Hz, 3H). ESI-MS m/z calc. 235.19362, found 236.2 (M+1)⁺; Retention time: 1.3 minutes; LC method I.

### Step 8: 2-Amino-4,4-dimethyl-hexan-1-ol

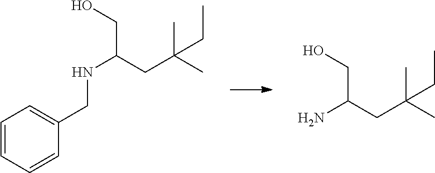

To a degassed solution of 2-(benzylamino)-4,4-dimethyl-hexan-1-ol (560 mg, 2.2603 mmol) in methanol (17 mL) was added 10 wt % palladium on carbon (50% wet) (400 mg, 0.1879 mmol). The mixture was purged with nitrogen for 5 minutes and then hydrogen was bubbled into the solution for 10 minutes. The mixture was then stirred under a hydrogen atmosphere (1 atm.) for 22 hours. The mixture was filtered through a short pad of Celite, the pad was rinsed with methanol (15 mL), and the filtrate was concentrated under reduced pressure to give 2-amino-4,4-dimethyl-hexan-1-ol (305 mg, 88%) as a colorless oil. ¹H NMR (400 MHz, CDCl₃) δ 3.55-3.47 (m, 1H), 3.18 (dd, J 10.3, 8.8 Hz, 1H), 2.98-2.88 (m, 1H), 1.36-1.21 (m, 4H), 1.13 (dd, J 14.5, 6.7 Hz, 1H), 0.91-0.90 (m, 6H), 0.84 (t, J 7.6 Hz, 3H); Two labile protons missing. ESI-MS m/z calc. 145.14667, found 146.2 (M+1)⁺; Retention time: 0.98 minutes; LC method I.

### Step 9: 3-[[4-(2-Amino-4,4-dimethyl-hexoxy)-6-(2,6-dimethylphenyl)pyrimidin-2-yl]sulfamoyl]benzoic acid

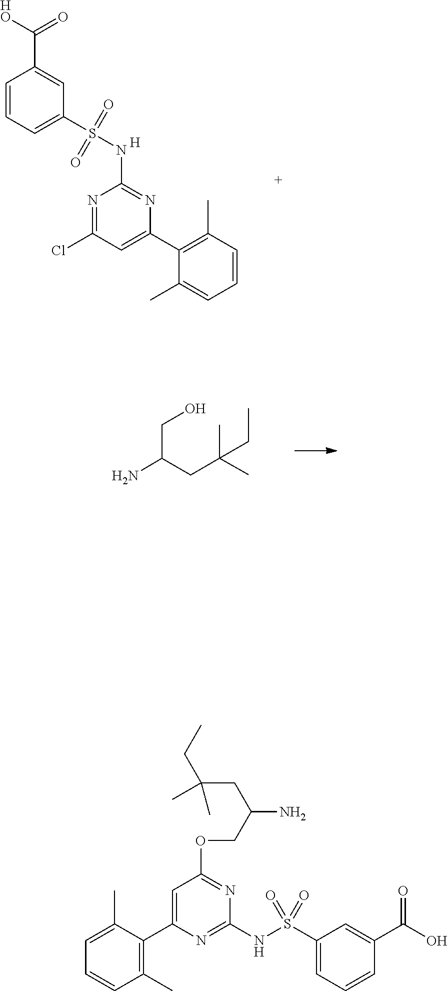

To a solution of 3-[[4-chloro-6-(2,6-dimethylphenyl)pyrimidin-2-yl]sulfamoyl]benzoic acid (870 mg, 2.0820 mmol) and 2-amino-4,4-dimethyl-hexan-1-ol (302 mg, 1.9753 mmol) in 2-methyltetrahydrofuran (8 mL) and N,N-dimethylformamide (0.8 mL) was added sodium tert-butoxide (800 mg, 8.3244 mmol) at 10-15° C. The mixture was stirred for 1 hour. More 3-[[4-chloro-6-(2,6-dimethylphenyl)pyrimidin-2-yl]sulfamoyl]benzoic acid (246 mg, 0.5887 mmol) and sodium tert-butoxide (225 mg, 2.3412 mmol) were added and the mixture was stirred for 45 minutes. The reaction was quenched with 1 N hydrochloric acid (20 mL) and extracted with 2-methyltetrahydrofuran (3×20 mL). The combined organic layers were dried over sodium sulfate, filtered and concentrated under reduced pressure. The crude was purified by reverse phase chromatography (Column: 80 g Cis. Gradient: 5-100% MeCN in water with 0.1% hydrochloric acid). The fraction containing the product were concentrated to remove acetonitrile, acidified to pH 1-2 with 1 N hydrochloric acid and extracted with 2-methyltetrahydrofuran (3×40 mL). The combined organic layers were dried over sodium sulfate, filtered, and concentrated under reduced pressure. The residue was triturated with ethyl acetate (30 mL), filtered and the precipitate was washed with ethyl acetate (2×15 mL). The remaining solid was lyophilized in 1:5 MeCN/water (25 mL) to give 3-[[4-(2-amino-4,4-dimethyl-hexoxy)-6-(2,6-dimethylphenyl)pyrimidin-2-yl]sulfamoyl]benzoic acid (hydrochloride salt) (670 mg, 59%) as a white powder. ¹H NMR (400 MHz, DMSO-d6) δ 12.99 (br. s., 1H), 8.46 (t, J 1.6 Hz, 1H), 8.36-7.84 (m, 4H), 7.68 (t, J 7.8 Hz, 1H), 7.26 (t, J 7.3 Hz, 1H), 7.13 (d, J 7.6 Hz, 2H), 6.28 (br. s., 1H), 4.32 (dd, J 11.6, 3.1 Hz, 1H), 4.10 (dd, J=11.9, 7.0 Hz, 1H), 3.61-3.53 (m, 1H), 2.01 (br. s., 6H), 1.58 (dd, J 14.7, 7.3 Hz, 1H), 1.47 (dd, J 14.7, 3.7 Hz, 1H), 1.24 (q, J 7.6 Hz, 2H), 0.88 (s, 3H), 0.87 (s, 3H), 0.79 (t, J 7.5 Hz, 3H); two labile protons missing. ESI-MS m/z calc. 526.225, found 527.1 (M+1)⁺; Retention time: 2.54 minutes; LC method J.

### Example 18: Preparation of 3-[[4-[(2R)-2-amino-3-cyclohexyl-propoxy]-6-(2,6-dimethylphenyl)pyrimidin-2-yl]sulfamoyl]benzoic acid

### Step 1: 3-[[4-[(2R)-2-Amino-3-cyclohexyl-propoxy]-6-(2,6-dimethylphenyl)pyrimidin-2-yl]sulfamoyl]benzoic acid

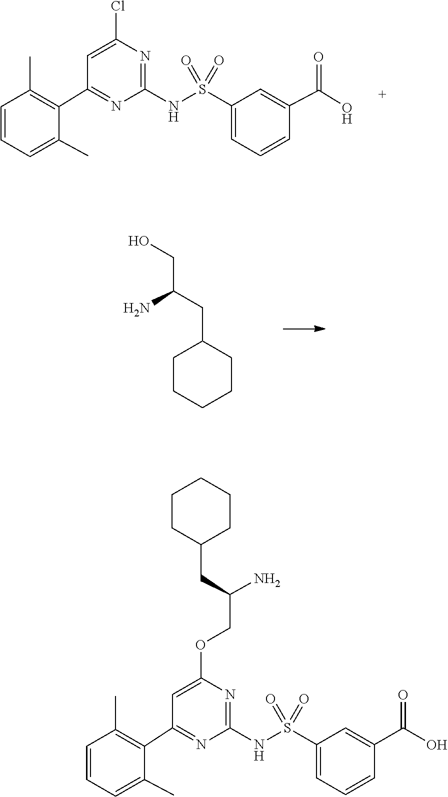 [SMILES: Cc1cccc(C)c1-c1cc(Cl)nc(NS(=O)(=O)c2cccc(C(=O)O)c2)n1 ; N[C@@H](CO)CC1CCCCC1 ; Cc1cccc(C)c1-c1cc(OC[C@H](N)CC2CCCCC2)nc(NS(=O)(=O)c2cccc(C(=O)O)c2)n1]

3-[[4-chloro-6-(2,6-Dimethylphenyl)pyrimidin-2-yl]sulfamoyl]benzoic acid (500 mg, 1.197 mmol) was combined with (2R)-2-amino-3-cyclohexyl-propan-1-ol (198 mg, 1.259 mmol) in THE (2.5 mL) and stirred vigorously for 5 minutes at room temperature. Sodium tert-butoxide (600 mg, 6.243 mmol) was added in one portion and the reaction mixture became very warm to the touch. Stirring was continued without any external heating for 15 minutes. The reaction mixture was then partitioned between 1M HCl and ethyl acetate. The layers were separated and the aqueous was extracted an additional 2× with ethyl acetate. The aqueous was diluted with an equal volume of brine and extracted an additional time with ethyl acetate. The combined organics were washed with brine, dried over sodium sulfate, and concentrated to give as an off white solid, 3-[[4-[(2R)-2-amino-3-cyclohexyl-propoxy]-6-(2,6-dimethylphenyl)pyrimidin-2-yl]sulfamoyl]benzoic acid (hydrochloride salt) (699.7 mg, 83%). ESI-MS m/z calc. 538.225, found 539.5 (M+1)⁺; Retention time: 0.51 minutes; LC method A.

### Example 19: Preparation of 3-[[4-[(2R)-2-amino-3-cyclopentyl-propoxy]-6-(2,6-dimethylphenyl)pyrimidin-2-yl]sulfamoyl]benzoic acid

### Step 1: (2R)-2-Amino-3-cyclopentyl-propan-1-ol

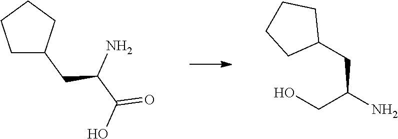 [SMILES: N[C@H](CC1CCCC1)C(=O)O ; N[C@@H](CO)CC1CCCC1]

To a stirred suspension of (2R)-2-amino-3-cyclopentyl-propanoic acid (1.00 g, 6.361 mmol) in anhydrous tetrahydrofuran (12 mL) was added borane-tetrahydrofuran (15 mL of 1.0 M, 15.00 mmol) at 0-4° C. (ice-water bath) the reaction mixture was left at room temperature for 20 h. The heterogenous mixture turned to a clear solution. Then hydrochloric acid (30 mL of 1.0 M, 30.00 mmol) was added slowly at 0-4° C. (ice-water bath). The reaction mixture was stirred for 45 min at ambient temperature. The volatiles were removed under reduced pressure. The solid residue was treated with aqueous sodium hydroxide (12.72 mL of 1.0 M, 12.72 mmol) The aqueous phase was separated and extracted with ethyl acetate (2×30 mL). The combined organic phases were washed with brine (50 mL), dried with magnesium sulfate, filtered, and concentrated under reduced pressure to obtain a semisolid. The latter was taken up in dichloromethane (20 mL) and treated with hydrogen chloride (4 M in dioxane) (5 mL of 4 M, 20.00 mmol) carefully at 0° C., and stirred for 1 h. The volatiles were removed under reduced pressure and triturated with hexanes. Then removal of the volatiles under reduced pressure and further drying furnished (2R)-2-amino-3-cyclopentyl-propan-1-ol (hydrochloride salt) (1.520 g, 133%) as a white solid. ¹H NMR (400 MHz, DMSO) δ 7.82 (s, 3H), 5.26 (t, J 5.3 Hz, 1H), 4.37 (s, 1H), 3.64-3.57 (m, 1H), 3.46-3.41 (m, 1H), 3.03 (s, 1H), 1.80-1.71 (m, 2H), 1.61-1.54 (m, 3H), 1.47-1.43 (m, 3H), 1.05 (qd, J=7.8, 3.7 Hz, 2H). ESI-MS m/z calc. 143.13101, found 142.2 (M+1)⁺; Retention time: 0.69 minutes; LC method A (1-50% gradient of MeCN).

### Step 2: 3-[[4-[(2R)-2-Amino-3-cyclopentyl-propoxy]-6-(2,6-dimethylphenyl)pyrimidin-2-yl]sulfamoyl]benzoic acid

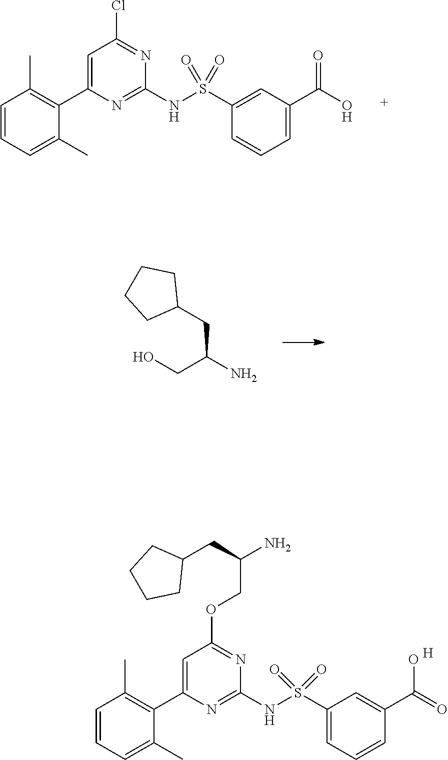

To a stirred mixture of 3-[[4-chloro-6-(2,6-dimethylphenyl)pyrimidin-2-yl]sulfamoyl]benzoic acid (1.0 g, 2.393 mmol) and (2R)-2-amino-3-cyclopentyl-propan-1-ol (hydrochloride salt) (0.55 g, 3.061 mmol) in anhydrous tetrahydrofuran (15 mL) nitrogen was purged for 5 min. Then solid sodium tert-butoxide (1.20 g, 10.08 mmol) was added at once. The heterogeneous mixture was stirred at ambient temperature for 20 h (overnight). The reaction was quenched by addition of cold hydrochloric acid (20 mL of 1 M, 20.00 mmol) and extracted with ethyl acetate (3×25 mL). The combined organics were washed with brine (20 mL), dried over sodium sulfate, filtered, and concentrated under reduced pressure. The crude was purified by preparative reverse-phase HPLC (1-99% acetonitrile in water over 15 min, 5 mM HCl as modifier) to furnish 3-[[4-[(2R)-2-amino-3-cyclopentyl-propoxy]-6-(2,6-dimethylphenyl)pyrimidin-2-yl]sulfamoyl]benzoic acid (hydrochloride salt) (387 mg, 29%) as a colorless solid. ESI-MS m/z calc. 524.20935, found 525.1 (M+1)⁺; Retention time: 1.12 minutes; LC method A.

### Example 20: Preparation of 3-[[4-[2-amino-4-(1-methylcyclopropyl)butoxy]-6-(2,6-dimethylphenyl)pyrimidin-2-yl]sulfamoyl]benzoic acid

### Step 1: Methyl 2-(tert-butoxycarbonylamino)-4-(I-methylcyclopropyl)but-2-enoate

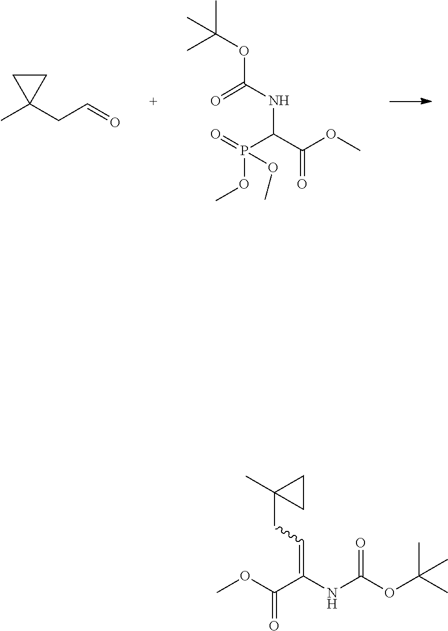

Nitrogen was bubbled to the crude solution of 2-(1-methylcyclopropyl)acetaldehyde (9.8 g, 99.854 mmol) in dichloromethane. Then methyl 2-(tert-butoxycarbonylamino)-2-dimethoxyphosphoryl-acetate (14.8 g, 49.791 mmol) was added and the mixture was cooled down to 0° C. after which time 1,8-diazabicyclo[5.4.0]undec-7-ene (38.684 g, 38 mL, 254.10 mmol) was added dropwise over 15 minutes. The reaction was stirred at 0° C. for 30 minutes then stirred overnight at room temperature. The reaction was quenched by the addition of a saturated aqueous solution of ammonium chloride (150 mL). The biphasic mixture was vigorously stirred for 30 minutes then the layers were separated, and the aqueous layer was extracted with dichloromethane (3×50 mL). The combined organic layers were washed with brine (200 mL), dried over magnesium sulfate, filtered and concentrated under reduced pressure to afford a dark brown oil which was purified by flash chromatography on silica gel (column: 120 g HP Gold; gradient: 0 to 40% ethyl acetate in heptanes, 15 CV). The desired fractions were concentrated under reduced pressure to afford methyl 2-(tert-butoxycarbonylamino)-4-(1-methylcyclopropyl)but-2-enoate (11.370 g, 81%) as a pale yellow oil which crystallized upon standing. ¹H NMR (400 MHz, CDCl₃) δ 6.68 (t, J 7.1 Hz, 1H), 5.95 (br. s, 1H), 3.79 (s, 3H), 2.14 (d, J 7.1 Hz, 2H), 1.47 (s, 9H), 1.04 (s, 3H), 0.40-0.35 (m, 2H), 0.34-0.29 (m, 2H). ESI-MS m/z calc. 269.1627, found 214.2 (M-55)⁺; Retention time: 1.87 minutes; LC method I.

### Step 2: Methyl 2-(tert-butoxycarbonylamino)-4-(1-methylcyclopropyl)butanoate

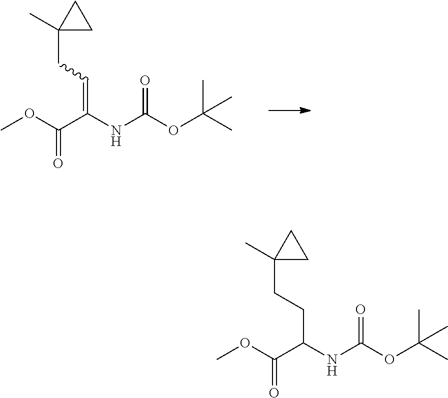 [SMILES: COC(=O)C(=CCC1(C)CC1)NC(=O)OC(C)(C)C ; COC(=O)C(CCC1(C)CC1)NC(=O)OC(C)(C)C]

To a stirred solution of methyl 2-(tert-butoxycarbonylamino)-4-(1-methylcyclopropyl)but-2-enoate (11.33 g, 39.963 mmol) in methanol (130 mL) was added nickel (II) chloride hexahydrate (9.5 g, 39.968 mmol). The green suspension was stirred for 15 minutes at room temperature until a green homogeneous solution was formed. Then a solution of sodium borohydride (15.2 g, 401.77 mmol) in methanol (130 mL) was added. The resulting black suspension was stirred at room temperature for 2 hours. The reaction was then quenched by the addition of water (200 mL). The aqueous layer was extracted with ethyl acetate (5×250 mL) and the combined organic layers were washed with water (500 mL), brine (500 mL), dried over sodium sulfate, filtered and concentrated under reduced pressure to afford crude methyl 2-(tert-butoxycarbonylamino)-4-(1-methylcyclopropyl)butanoate (10.54 g, 92%) as a colorless oil which was used in the next step without further purification. ¹H NMR (400 MHz, CDCl₃) δ 4.96 (d, J 7.1 Hz, 1H), 4.35-4.24 (m, 1H), 3.74 (s, 3H), 1.99-1.84 (m, 1H), 1.77-1.63 (m, 1H), 1.45 (s, 9H), 1.30-1.21 (m, 2H), 1.01 (s, 3H), 0.32-0.15 (m, 4H). ESI-MS m/z calc. 271.1784, found 172.2 (M-99)+; Retention time: 1.92 minutes; LC method I.

### Step 3: tert-Butyl N-[1-(hydroxymethyl)-3-(I-methylcyclopropyl)propyl]carbamate

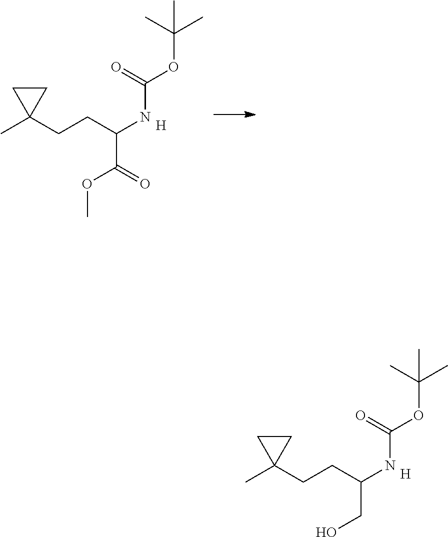

A solution of crude methyl 2-(tert-butoxycarbonylamino)-4-(1-methylcyclopropyl) butanoate (3.41 g, 11.586 mmol) was dissolved in anhydrous tetrahydrofuran (70 mL) under nitrogen atmosphere and was then cooled down to 0° C. Lithium borohydride (650 mg, 26.855 mmol) was added to the solution and the reaction was stirred at 0° C. for 15 minutes then stirred at room temperature for 2 hours. Supplementary lithium borohydride (650 mg, 26.855 mmol) was added, and the reaction was stirred at room temperature for additional 3 hours after which time another batch of lithium borohydride (650 mg, 26.855 mmol) was added. The reaction was then stirred overnight at room temperature then added to a saturated aqueous solution of ammonium chloride (250 mL). The biphasic mixture was vigorously stirred at room temperature for 30 minutes and the layers were then separated. The aqueous layer was extracted with ethyl acetate (5×50 mL) and the combined organic layers were washed with brine, dried over sodium sulfate, filtered and concentrated under reduced pressure to afford crude tert-butyl N-[1-(hydroxymethyl)-3-(1-methylcyclopropyl)propyl]carbamate (3.09 g, 101%) as a colorless oil which was directly used in the next step without further purification ¹H NMR (400 MHz, CDCl₃) δ 4.58 (br. s, 1H), 3.72-3.49 (m, 3H), 2.42 (br. s, 1H), 1.68-1.57 (m, 1H), 1.54-1.40 (m, 10H), 1.37-1.20 (m, 2H), 1.02 (s, 3H), 0.30-0.19 (m, 4H). ESI-MS m/z calc. 243.1834, found 266.2 (M+23)+; 188.2 (M-55)+; Retention time: 1.75 minutes; LC method I.

### Step 4: 2-Amino-4-(I-methylcyclopropyl)butan-1-ol

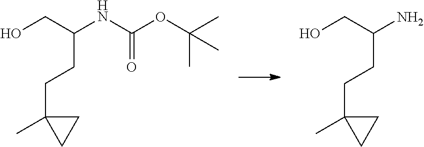

To a stirred solution of tert-butyl N-[1-(hydroxymethyl)-3-(1-methylcyclopropyl) propyl]carbamate (882 mg, 3.6245 mmol) in dichloromethane (20 mL) at 0° C. was added dropwise a solution of hydrogen chloride 4 M in dioxane (10 mL of 4 M, 40.000 mmol). The reaction was stirred for 30 minutes at 0° C. then stirred overnight at room temperature. The reaction was then concentrated under reduced pressure and the solid was co-evaporated with methanol (3×10 mL) then freeze-dried to afford 2-amino-4-(1-methylcyclopropyl)butan-1-ol (hydrochloride salt) (604 mg, 88%) as an off-white powder which could be used in the next step without further purification. ¹H NMR (400 MHz, DMSO-d6) δ 7.89 (br. s, 3H), 5.34-5.22 (m, 1H), 3.62-3.51 (m, 1H), 3.47-3.36 (m, 1H), 3.06-2.93 (m, 1H), 1.65-1.52 (m, 2H), 1.30-1.19 (m, 2H), 0.99 (s, 3H), 0.31-0.15 (m, 4H). ESI-MS m/z calc. 143.13101, found 144.2 (M+1)⁺; Retention time: 0.81 minutes; LC method I.

### Step 5: 3-[[4-[2-Amino-4-(I-methylcyclopropyl)butoxy]-6-(2,6-dimethylphenyl)pyrimidin-2-yl]sulfamoyl]benzoic acid

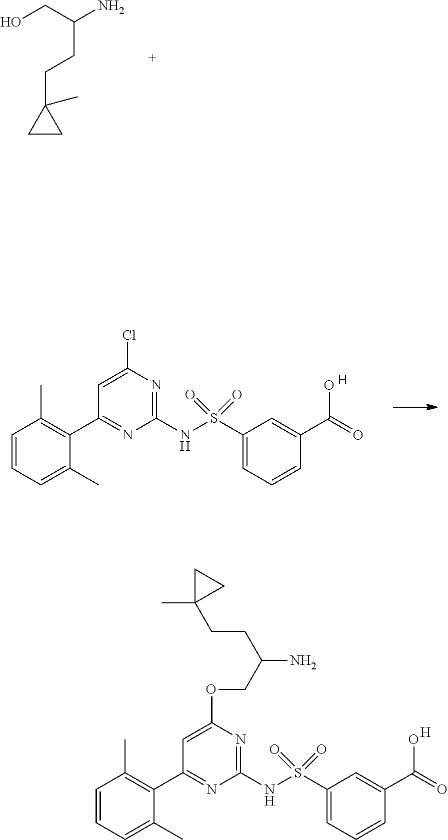

A solution of 2-amino-4-(1-methylcyclopropyl)butan-1-ol (hydrochloride salt) (604 mg, 3.3614 mmol) in anhydrous N,N-dimethylformamide (3.5 mL) was added to a solution of 3-[[4-chloro-6-(2,6-dimethylphenyl)pyrimidin-2-yl]sulfamoyl]benzoic acid (1.2 g, 2.8717 mmol) in 2-methyltetrahydrofuran (35 mL). The reaction was cooled down to 10-15° C. then sodium tert-butoxide (700 mg, 7.2838 mmol) was added. The reaction was stirred for 30 minutes at 10-15° C. then supplementary sodium tert-butoxide (700 mg, 7.2838 mmol) was added. The reaction was then stirred for 1 hour then cooled down to 0° C. and quenched by the addition of an aqueous solution of 1 N hydrochloric acid (40 mL). The biphasic mixture was stirred for 30 minutes then layers were separated, and the aqueous layer was extracted with 2-methyltetrahydrofuran (5×30 mL). The combined organic layers were washed with brine (150 mL), dried over magnesium sulfate then concentrated under reduced pressure to afford a pale yellow fluffy solid which was purified by reverse-phase chromatography (column: 120 g Cis; gradient: 10 to 45% methanol in water containing 0.1% v/v of hydrochloric acid; 35 CV). The desired fractions were concentrated under reduced pressure and the residual water was co-evaporated with methanol (6×10 mL) and the residue was freeze-dried to afford 3-[[4-[2-amino-4-(1-methylcyclopropyl)butoxy]-6-(2,6-dimethylphenyl)pyrimidin-2-yl]sulfamoyl]benzoic acid (hydrochloride salt) (586 mg, 29%) as a white fluffy solid. ¹H NMR (400 MHz, DMSO-d6) δ 13.39 (br. s, 1H), 8.49-8.41 (m, 1H), 8.16-8.03 (m, 5H), 7.69 (t, J 7.9 Hz, 1H), 7.29-7.20 (m, 1H), 7.15-7.09 (m, 2H), 6.31 (br. s, 1H), 4.38 (dd, J 12.0, 3.2 Hz, 1H), 4.25 (dd, J 11.9, 6.2 Hz, 1H), 3.55-3.48 (m, 1H, overlapped with water), 2.00 (br. s, 6H), 1.73-1.63 (m, 2H), 1.37-1.22 (m, 2H), 0.99 (s, 3H), 0.37-0.10 (m, 4H). (1H missing, labile proton). ESI-MS m/z calc. 524.20935, found 525.1 (M+1)⁺; Retention time: 2.53 minutes; LC method J.

### Example 21: Preparation of 3-[[4-[2-Amino-2-(3,3-dimethylcyclobutyl)ethoxy]-6-(2,6-dimethylphenyl)pyrimidin-2-yl]sulfamoyl]benzoic acid

### Step 1: N-Methoxy-N,3,3-trimethyl-cyclobutanecarboxamide

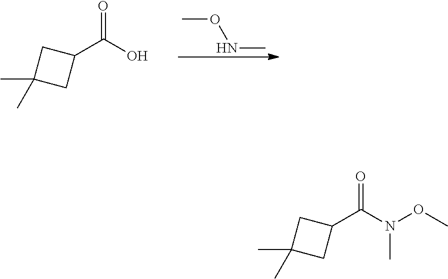

To a solution of 3,3-dimethylcyclobutanecarboxylic acid (2 g, 15.136 mmol), N-methoxymethanamine (hydrochloride salt) (3.01 g, 30.241 mmol) and HATU (6.44 g, 16.598 mmol) in DMF (30 mL) at 0° C. was added triethylamine (4.6464 g, 6.4 mL, 45.918 mmol). The reaction mixture was warmed to room temperature was stirred for 21 hours. The mixture was diluted with water (40 mL) and mixture was extracted with EtOAc (3×40 mL). The combined organic layers were washed with 1 N hydrochloric acid solution (2×30 mL), saturated solution of sodium bicarbonate (2×30 mL) and brine (20 mL), dried over sodium sulfate, filtered, and concentrated under reduced pressure to afford N-methoxy-N,3,3-trimethyl-cyclobutanecarboxamide (2.72 g, 99%) as a colorless oil. ¹H NMR (400 MHz, CDCl₃) δ 3.65 (s, 3H), 3.49-3.31 (m, 1H), 3.18 (s, 3H), 2.15-2.07 (m, 2H), 1.95-1.87 (m, 2H), 1.20 (s, 3H), 1.09 (s, 3H). ESI-MS m/z calc. 171.12593, found 172.2 (M+1)⁺; Retention time: 1.67 minutes; LC method I.

### Step 2: 3,3-Dimethylcyclobutanecarbaldehyde

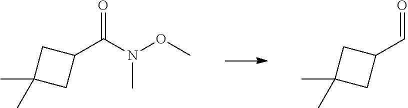

To a suspension of lithium aluminum hydride (860 mg, 22.659 mmol) in dry THE (15 mL) at 0° C. was added dropwise a solution of N-methoxy-N,3,3-trimethyl-cyclobutanecarboxamide (2.72 g, 14.931 mmol) in dry THE (15 mL). The mixture was stirred 5 min at 0° C. then allowed to reach rt and stirred for 2 h. The mixture was cooled down to 0° C., quenched with water (50 mL) and extracted with MTBE (3×50 mL). The combined organic layers were washed with brine (2×40 mL), dried over sodium sulfate, and filtered to give crude 3,3-dimethylcyclobutanecarbaldehyde (1.6748 g, 100%) as a colorless solution in MTBE/THF.

### Step 3: 2-(Benzylamino)-2-(3,3-dimethylcyclobutyl)acetonitrile

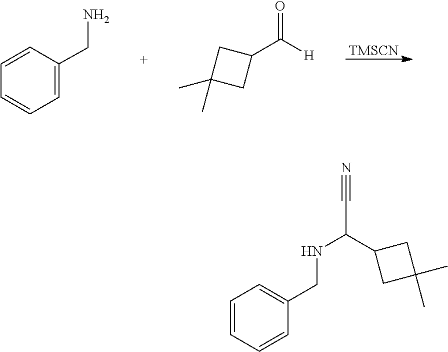

A three neck round bottom flask equipped with a 6 N NaOH trap, was charged with crude 3,3-dimethylcyclobutanecarbaldehyde (1.675 g, 14.933 mmol) as a 5:1 MTBE/THF solution (˜180 mL) and benzylamine (1.8149 g, 1.85 mL, 16.937 mmol). To the mixture was slowly added at 0° C. acetic acid (971.52 mg, 0.92 mL, 16.178 mmol) and then trimethylsilyl cyanide (1.5146 g, 1.91 mL, 15.267 mmol). The mixture was warmed to room temperature and stirred for 18 hours. The mixture was diluted with water (150 mL) and extracted with ethyl acetate (2×100 mL). The combined organic layers were washed with brine (2×60 mL), dried over anhydrous sodium sulfate, filtered, and concentrated under reduced pressure. Purification by chromatography on silica gel (Column: 120 g. Gradient: 0-20% ethyl acetate in heptanes) afforded 2-(benzylamino)-2-(3,3-dimethylcyclobutyl)acetonitrile (1.66 g, 47%) as a light yellow oil. ¹H NMR (400 MHz, CDCl₃) δ 7.38-7.27 (m, 5H), 4.07 (d, J 13.0 Hz, 1H), 3.83 (d, J 13.0 Hz, 1H), 3.46 (d, J 7.1 Hz, 1H), 2.66-2.53 (m, 1H), 1.97-1.85 (m, 2H), 1.79-1.64 (m, 2H), 1.16 (s, 3H), 1.09 (s, 3H); 1 labile proton missing. ESI-MS m/z calc. 228.16264, found 229.2 (M+1)⁺; Retention time: 1.9 minutes; LC method I.

### Step 4: 2-(Benzylamino)-2-(3,3-dimethylcyclobutyl)acetic acid

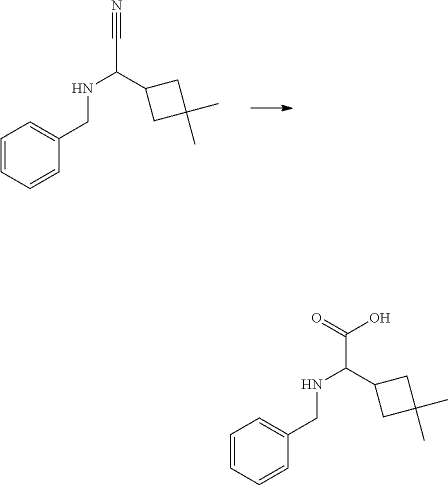 [SMILES: CC1(C)CC(C(C#N)NCc2ccccc2)C1 ; CC1(C)CC(C(NCc2ccccc2)C(=O)O)C1]

2-(Benzylamino)-2-(3,3-dimethylcyclobutyl)acetonitrile (1.66 g, 7.0229 mmol) was dissolved in acetic acid (9.8208 g, 9.3 mL, 163.54 mmol) and hydrochloric acid (56 mL of 12 M, 672.00 mmol). The mixture was stirred at 100° C. for 18 hours. More acetic acid (3.1680 g, 3 mL, 52.754 mmol) and hydrochloric acid (20 mL of 12 M, 240.00 mmol) were added, and the mixture was stirred at 100° C. for 20 hours. The solution was cooled to 10-15° C., the pH was increased to 3-4 with saturated sodium bicarbonate aqueous solution, the mixture was filtered, the solid was washed with water (15 ml) and dried under vacuum to afford 2-(benzylamino)-2-(3,3-dimethylcyclobutyl)acetic acid (1.486 g, 85%) as a grey solid. ¹H NMR (400 MHz, MeOH-d4) δ 7.52-7.40 (m, 5H), 4.19 (d, J 13.0 Hz, 1H), 4.09 (d, J 13.0 Hz, 1H), 3.38 (d, J 8.3 Hz, 1H), 2.70-2.55 (m, 1H), 1.96-1.75 (m, 4H), 1.16 (s, 3H), 1.05 (s, 3H); Two labile protons missing. ESI-MS m/z calc. 247.15723, found 248.2 (M+1)⁺; Retention time: 1.3 minutes; LC method I.

### Step 5: 2-(Benzylamino)-2-(3,3-dimethylcyclobutyl)ethanol

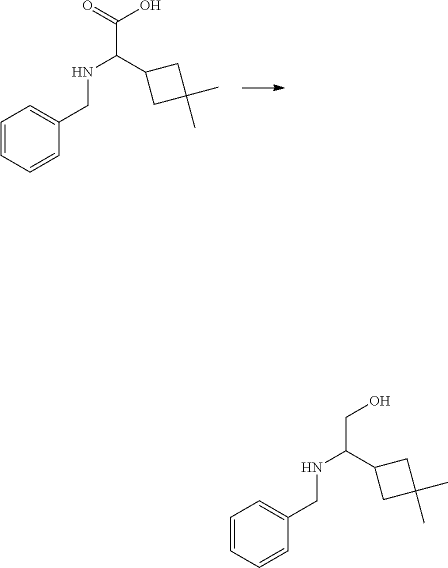 [SMILES: CC1(C)CC(C(NCc2ccccc2)C(=O)O)C1 ; CC1(C)CC(C(CO)NCc2ccccc2)C1]

To a solution of 2-(benzylamino)-2-(3,3-dimethylcyclobutyl)acetic acid (1.486 g, 6.0021 mmol) in anhydrous THF (23 mL) was added drop wise at 0° C. borane tetrahydrofuran complex solution in THE (19.4 mL of 1 M, 19.400 mmol). The reaction was stirred for 30 minutes at 0° C. and then at room temperature for 18 hours. The reaction was cooled down to 0° C. then quenched by the slow addition of methanol (25 mL) and concentrated under reduced pressure. The resulting residue was partitioned between ethyl acetate (50 mL) and 1M sodium hydroxide aqueous solution (50 mL). The biphasic mixture was vigorously stirred until complete solubilization, the layers were separated, and the aqueous layer was extracted ethyl acetate (3×40 mL). The combined organic layers were washed with brine (40 mL), dried over anhydrous sodium sulfate, filtered and concentrated under reduced pressure to afford 2-(benzylamino)-2-(3,3-dimethylcyclobutyl)ethanol (1.07 g, 69%) a colorless oil. ¹H NMR (400 MHz, CDCl₃) δ 7.45-7.28 (m, 5H), 3.83-3.73 (m, 2H), 3.59 (dd, J 10.5, 3.7 Hz, 1H), 3.27 (dd, J 10.6, 4.5 Hz, 1H), 2.60 (dt, J 9.8, 4.2 Hz, 1H), 2.36 (dq, J 17.9, 8.8 Hz, 1H), 1.92-1.80 (m, 2H), 1.54-1.43 (m, 3H), 1.14 (s, 3H), 1.04 (s, 3H); One labile proton missing. ESI-MS m/z calc. 233.17796, found 234.2 (M+1)⁺; Retention time: 1.27 minutes; LC method I.

### Step 6: 2-Amino-2-(3,3-dimethylcyclobutyl)ethanol

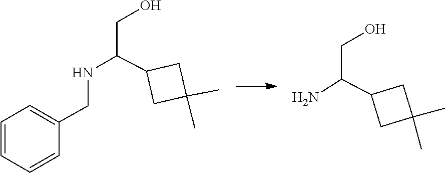

To a degassed solution of 2-(benzylamino)-2-(3,3-dimethylcyclobutyl)ethanol (1.07 g, 4.1269 mmol) in methanol (18 mL) was added 10 wt % palladium on carbon (50% wet) (455 mg, 0.2138 mmol). The mixture was purged with nitrogen for 5 minutes and then hydrogen was bubbled into the solution for 10 minutes. The mixture was then stirred under a hydrogen atmosphere (1 atm.) for 18 hours. The mixture was filtered through a short pad of Celite, the pad was rinsed with methanol (30 mL), and the filtrate was concentrated under reduced pressure to give 2-amino-2-(3,3-dimethylcyclobutyl)ethanol (632 mg, 102%) as a colorless oil. ¹H NMR (400 MHz, CDCl₃) δ 3.55 (dd, J 10.5, 3.4 Hz, 1H), 3.17 (dd, J 10.5, 7.8 Hz, 1H), 2.75-2.66 (m, 1H), 2.17-2.03 (m, 1H), 1.90-1.73 (m, 2H), 1.58-1.43 (m, 2H; overlapped with water), 1.15 (s, 3H), 1.06 (s, 3H); 3 labile protons missing. ESI-MS m/z calc. 143.13101, found 144.2 (M+1)⁺; Retention time: 0.83 minutes; LC method I.

### Step 7: 3-[[4-[2-Amino-2-(3,3-dimethylcyclobutyl)ethoxy]-6-(2,6-dimethylphenyl)pyrimidin-2-yl]sulfamoyl]benzoic acid

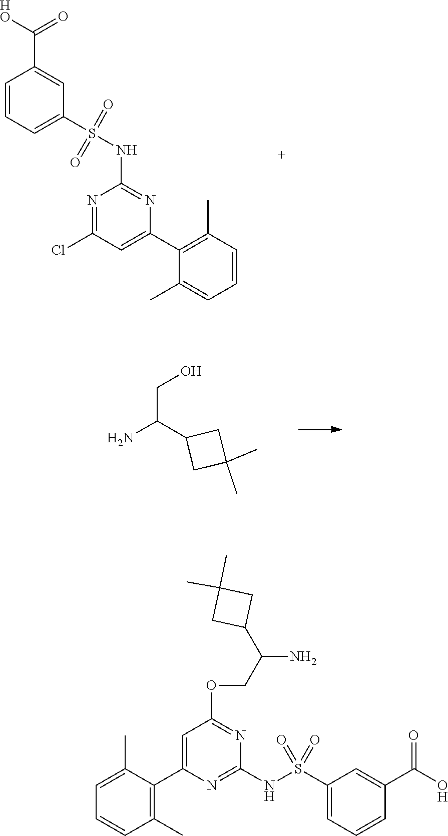

To a solution of 3-[[4-chloro-6-(2,6-dimethylphenyl)pyrimidin-2-yl]sulfamoyl]benzoic acid (1.74 g, 4.1640 mmol) and 2-amino-2-(3,3-dimethylcyclobutyl)ethanol (630 mg, 4.1787 mmol) in 2-methyltetrahydrofuran (17 mL) and N,N-dimethylformamide (1.7 mL) was added sodium tert-butoxide (1.7 g, 17.689 mmol) at 10-15° C. and then the mixture was stirred for 1 hour. The reaction was quenched with iN hydrochloric solution (50 mL) and extracted with 2-methyltetrahydrofuran (3×50 mL). The combined organic layers were washed with brine (40 mL), dried over anhydrous sodium sulfate, filtered and concentrated under reduced pressure. The residue was triturated with ethyl acetate (80 mL), the precipitate was collected by filtration, washed with ethyl acetate (30 mL) and dried to give 3-[[4-[2-amino-2-(3,3-dimethylcyclobutyl)ethoxy]-6-(2,6-dimethylphenyl)pyrimidin-2-yl]sulfamoyl]benzoic acid (hydrochloride salt) (1.453 g, 58%) as a white solid after lyophilization. ¹H NMR (400 MHz, DMSO-d6) δ 13.21 (br. s., 1H), 8.44 (t, J 1.6 Hz, 1H), 8.29-7.95 (m, 5H), 7.69 (t, J 7.8 Hz, 1H), 7.26 (t, J 7.6 Hz, 1H), 7.13 (d, J 7.6 Hz, 2H), 6.29 (br. s., 1H), 4.28 (dd, J 11.9, 2.8 Hz, 1H), 4.08 (dd, J 11.9, 6.5 Hz, 1H), 3.54-3.43 (m, 1H), 2.48-2.38 (m, 1H, overlapped with DMSO), 2.01 (br. s., 6H), 1.87-1.80 (m, 1H), 1.77-1.65 (m, 3H), 1.13 (s, 3H), 1.06 (s, 3H); one labile proton missing. ESI-MS m/z calc. 524.20935, found 525.1 (M+1)⁺; Retention time: 2.53 minutes; LC method J.

### Example 22: Preparation of 3-[[4-[2-amino-3-(3,3-dimethylcyclobutyl)propoxy]-6-(2,6-dimethylphenyl)pyrimidin-2-yl]sulfamoyl]benzoic acid

### Step 1: Methyl (E)-2-(tert-butoxycarbonylamino)-3-(3,3-dimethylcyclobutyl)prop-2-enoate

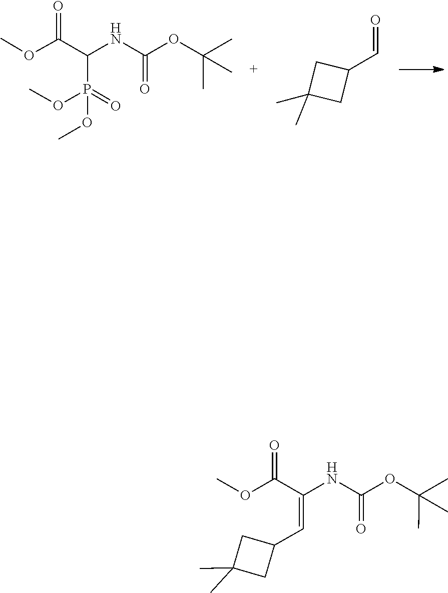

In a flame-dried flask under N₂ was placed 3,3-dimethylcyclobutanecarbaldehyde (1.6695 g, 14.884 mmol) (as solution in diethyl ether/THF), methyl 2-(tert-butoxycarbonylamino)-2-dimethoxyphosphoryl-acetate (1.806 g, 5.9543 mmol) and dry dioxane (119 mL). The solution was cooled to 0° C. and 1,1,3,3-tetramethylguanidine (3.5086 g, 3.9 mL, 29.853 mmol) was added dropwise. Reaction was slowly allowed to reach rt and stirred for 3 days. Water was added (150 mL) and the mixture was extracted with EtOAc (3×60 mL). Organic phase was washed with brine (50 mL), dried over sodium sulfate, filtered and concentrated under reduced pressure. Residue was purified by silica gel chromatography on a 25 g GOLD cartridge, eluting with a gradient of AcOEt in heptanes (0 to 15% over 18 CV). Desired fractions were combined and concentrated under reduced pressure to afford methyl (E)-2-(tert-butoxycarbonylamino)-3-(3,3-dimethylcyclobutyl)prop-2-enoate (1.817 g, 99%) as a white semi-solid. ¹H NMR (400 MHz, CDCl₃) δ 6.62 (d, J 8.1 Hz, 1H), 5.86 (br. s., 1H), 3.78 (s, 3H), 3.20 (sxt, J 8.6 Hz, 1H), 2.07-2.01 (m, 2H), 1.76-1.70 (m, 2H), 1.47 (s, 9H), 1.17 (s, 3H), 1.07 (s, 3H). ESI-MS m/z calc. 283.1784, found 306.2 (M+23)+; 228.2 (M-55)⁺; Retention time: 1.948 minutes; LC method I.

### Step 2: Methyl 2-(tert-butoxycarbonylamino)-3-(3,3-dimethylcyclobutyl)propanoate

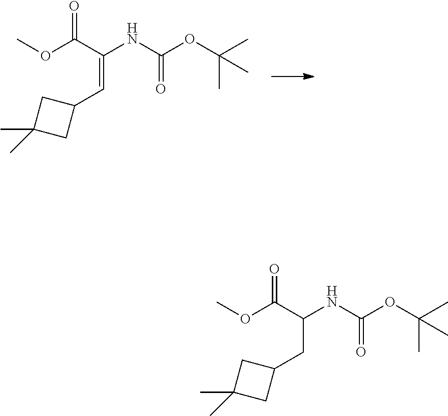 [SMILES: COC(=O)/C(=C\C1CC(C)(C)C1)NC(=O)OC(C)(C)C ; COC(=O)C(CC1CC(C)(C)C1)NC(=O)OC(C)(C)C]

In a flame-dried flask under N₂ was placed methyl (E)-2-(tert-butoxycarbonylamino)-3-(3,3-dimethylcyclobutyl)prop-2-enoate (1.817 g, 5.8993 mmol), palladium on carbon (628 mg, 10% w/w, 0.5901 mmol) and MeOH (23 mL). Hydrogen was bubbled into the suspension for 5 minutes. Reaction was then stirred under H₂ (1 atm.) for 19 h. Nitrogen was then bubbled into the mixture for 10 minutes. The reaction mixture was directly filtered over a pad of Celite with MeOH. Solution was filtered again over a pad of Celite then through a nylon 0.45 m filter to obtain a clear solution. The solvent was removed under reduced pressure to afford methyl 2-(tert-butoxycarbonylamino)-3-(3,3-dimethylcyclobutyl)propanoate (1.6016 g, 90%) as a pale yellow oil. ¹H NMR (400 MHz, CDCl₃) δ 4.94 (d, J 7.3 Hz, 1H), 4.28-4.16 (m, 1H), 3.73 (s, 3H), 2.27 (spt, J 8.1 Hz, 1H), 1.93-1.82 (m, 3H), 1.78-1.71 (m, 1H), 1.52-1.36 (m, 11H), 1.12 (s, 3H), 1.04 (s, 3H). ESI-MS m/z calc. 285.194, found 308.2 (M+23)+; 186.2 (M-99)+; Retention time: 1.999 minutes; LC method I.

### Step 3: tert-Butyl N-[1-[(3,3-dimethylcyclobutyl)methyl]-2-hydroxy-ethyl]carbamate

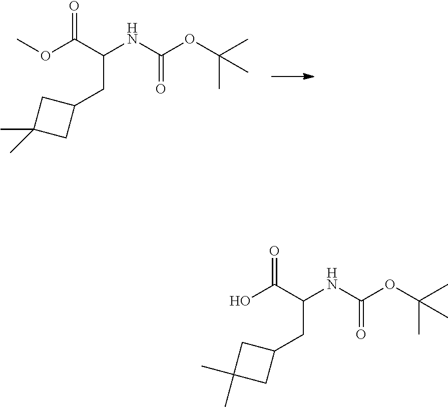 [SMILES: COC(=O)C(CC1CC(C)(C)C1)NC(=O)OC(C)(C)C ; CC1(C)CC(CC(NC(=O)OC(C)(C)C)C(=O)O)C1]

In a flame-dried flask under N₂ was placed methyl 2-(tert-butoxycarbonylamino)-3-(3,3-dimethylcyclobutyl)propanoate (1.6 g, 5.3263 mmol) and dry THE (20 mL). The resulting solution was cooled down to 0° C. and a solution of LiAlH₄ in THE (4.2 mL of 2 M, 8.4000 mmol) was added dropwise. The mixture was stirred 5 min at 0° C. then warmed to rt and stirred for 2 h. Mixture was cooled down to 0° C. Water (319 L) was added dropwise followed by NaOH 15% aqueous sol. (319 L) then water (957 L). Solution was allowed to reach rt and stirred for 30 min. Magnesium sulfate was then added to the mixture and stirring was continued for 15 min. Solution was directly filtered over a pad of Celite, washed with DCM and concentrated under reduced pressure to afford crude tert-butyl N-[1-[(3,3-dimethylcyclobutyl)methyl]-2-hydroxy-ethyl]carbamate (1.26 g, 83%) as a white solid. ¹H NMR (400 MHz, CDCl₃) δ 4.55 (br. s., 1H), 3.68-3.61 (m, 1H), 3.60-3.53 (m, 1H), 3.53-3.46 (m, 1H), 2.36 (br. s, 1H), 2.31-2.20 (m, 1H), 1.94-1.85 (m, 2H), 1.56-1.49 (m, 2H, overlapped with water), 1.49-1.37 (m, 11H), 1.13 (s, 3H), 1.04 (s, 3H). ESI-MS m/z calc. 257.1991, found 280.2 (M+23)+; 202.2 (M-55)⁺; Retention time: 1.842 minutes; LC method I.

### Step 4: 2-Amino-3-(3,3-dimethylcyclobutyl)propan-1-ol

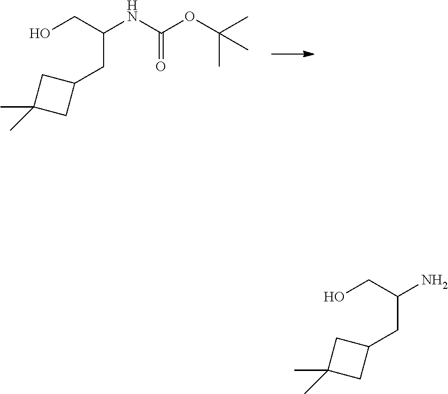 [SMILES: CC1(C)CC(CC(CO)NC(=O)OC(C)(C)C)C1 ; CC1(C)CC(CC(N)CO)C1]

In a reaction tube under N₂ was placed tert-butyl N-[1-[(3,3-dimethylcyclobutyl)methyl]-2-hydroxy-ethyl]carbamate (1.26 g, 4.6509 mmol) and dry DCM (18 mL). The resulting solution was cooled down to 0° C. and a solution of HCl in dioxane (11.6 mL of 4 M, 46.400 mmol) was added dropwise. The mixture was stirred 5 min at 0° C. then warmed to rt and stirred 16 h. Solution was concentrated under reduced pressure to afford crude 2-amino-3-(3,3-dimethylcyclobutyl)propan-1-ol (hydrochloride salt) (992.1 mg, 105%) as a light pink solid. ¹H NMR (400 MHz, DMSO-d6) δ 8.05-7.57 (m, 3H), 5.34-5.17 (m, 1H), 3.58-3.50 (m, 1H), 2.91 (br. s, 1H), 2.35-2.23 (m, 1H), 1.89-1.79 (m, 2H), 1.64-1.55 (m, 2H), 1.44-1.34 (m, 2H), 1.11 (s, 3H), 1.03 (s, 3H). (1H missing, labile proton) ESI-MS m/z calc. 157.14667, found 158.2 (M+1)⁺; Retention time: 1.158 minutes; LC method I.

### Step 5: 3-[[4-[2-Amino-3-(3,3-dimethylcyclobutyl)propoxy]-6-(2,6-dimethylphenyl)pyrimidin-2-yl]sulfamoyl]benzoic acid

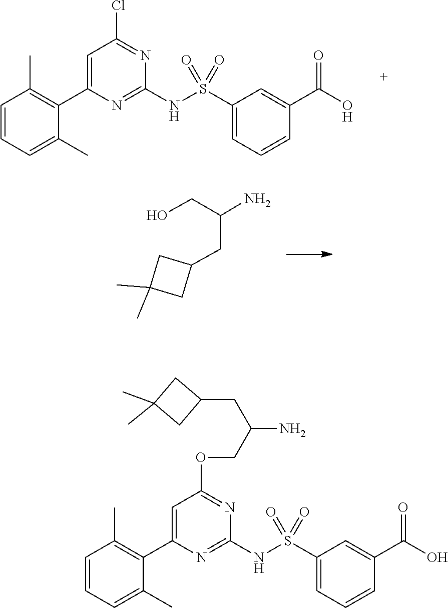

A solution of 2-amino-3-(3,3-dimethylcyclobutyl)propan-1-ol (hydrochloride salt) (990 mg, 4.8551 mmol) in dry DMF (3.3 mL) was added to a solution of 3-[[4-chloro-6-(2,6-dimethylphenyl)pyrimidin-2-yl]sulfamoyl]benzoic acid (2.13 g, 5.0973 mmol) in 2-MeTHF (29.7 mL). The mixture was then cooled down to 10-15° C. and sodium tert-butoxide (2.8 g, 28.261 mmol) was added. The reaction was stirred at 10-15° C. for 2 h then cooled down to 0° C. and quenched by the addition of an aqueous solution of 1N HCl (50 mL). The biphasic mixture was stirred for 15 minutes at 0° C. then concentrated under reduced pressure. Residue was purified by reverse phase chromatography on a 120 g C₁₈ GOLD cartridge, eluting with a gradient of MeCN in acidic water (0.1% v/v of HCl in water) (5% for 4 CV then 5 to 70% over 20 CV). Desired fractions were combined and concentrated under reduced pressure. Product was then lyophilized to afford 3-[[4-[2-amino-3-(3,3-dimethylcyclobutyl)propoxy]-6-(2,6-dimethylphenyl)pyrimidin-2-yl]sulfamoyl]benzoic acid (hydrochloride salt) (2.1251 g, 74%) as an off-white solid. ¹H NMR (400 MHz, DMSO-d6) δ 13.81-11.71 (m, 1H), 8.48-8.42 (m, 1H), 8.23-8.05 (m, 5H), 7.71 (t, J=7.8 Hz, 1H), 7.26 (t, J=7.6 Hz, 1H), 7.13 (d, J=7.6 Hz, 2H), 6.30 (br. s., 1H), 4.31 (dd, J=11.7, 2.9 Hz, 1H), 4.09 (dd, J=11.7, 6.1 Hz, 1H), 3.40-3.33 (m, 1H, overlapped with water), 2.35-2.23 (m, 1H), 2.10-1.93 (m, 6H, overlapped with MeCN), 1.86 (dd, J=10.9, 7.9 Hz, 1H), 1.81-1.65 (m, 3H), 1.44 (t, J=9.8 Hz, 1H), 1.37 (t, J=9.8 Hz, 1H), 1.10 (s, 3H), 1.02 (s, 3H). (1H missing, labile proton) ESI-MS m/z calc. 538.225, found 539.2 (M+1)⁺; Retention time. 2.642 minutes; LC method I.

### Example 23: Preparation of 3-[[4-[(2R)-2-amino-3-(cyclopropoxy)propoxy]-6-(2,6-dimethylphenyl)pyrimidin-2-yl]sulfamoyl]benzoic acid

### Step 1: O1-Benzyl O2-methyl(2R)-aziridine-1,2-dicarboxylate

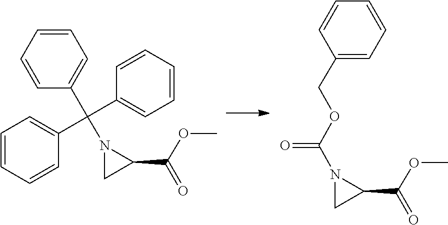 [SMILES: COC(=O)[C@H]1CN1C(c1ccccc1)(c1ccccc1)c1ccccc1 ; COC(=O)[C@H]1CN1C(=O)OCc1ccccc1]

TFA (35.520 g, 24 mL, 311.52 mmol) was added over a period of 10 minutes to a solution of methyl (2R)-1-tritylaziridine-2-carboxylate (6.6 g, 19.219 mmol) in a mixture of chloroform (24 mL) and methanol (24 mL) at 0° C. The reaction was stirred in an ice bath under nitrogen for 4 hours. The solvent was removed in vacuo at 0° C. The last traces of TFA were removed by azeotroping with diethyl ether (3×30 mL). The residue was partitioned between ether (30 mL) and water (30 mL). The ether layer was extracted with water (3×30 mL) and the combined aqueous extracts were made basic with sodium bicarbonate (10.5 g, 124.99 mmol). ethyl acetate (150 mL) was added to the aqueous layers and the mixture was cooled to 0° C. benzyl chloroformate (3.3600 g, 2.8 mL, 19.696 mmol) was added to the mixture, and the reaction was stirred at rt for 20 hours. The layers were separated, and the aqueous layer was extracted with ethyl acetate (3×50 mL). The combined organic layers were washed with brine (2×50 mL) and dried over anhydrous sodium sulfate. The solution was concentrated under vacuum to furnish O1-benzyl O2-methyl(2R)-aziridine-1,2-dicarboxylate (4.436 g, 98%) as a clear liquid. ¹H NMR (500 MHz, Chloroform-d) δ 7.47-7.27 (m, 5H), 5.15 (d, J 1.6 Hz, 2H), 3.71 (s, 3H), 3.11 (dd, J 5.5, 3.2 Hz, 1H), 2.60 (dd, J 3.2, 1.3 Hz, 1H), 2.48 (dd, J 5.5, 1.3 Hz, 1H).

### Step 2: Methyl (2R)-2-(benzyloxycarbonylamino)-3-(cyclopropoxy)propanoate

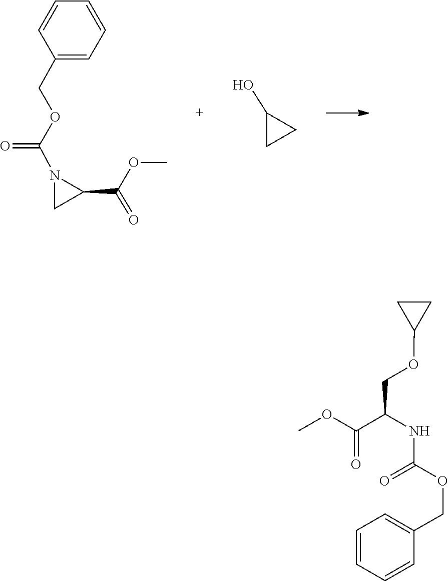

To a solution of O1-benzyl O2-methyl(2R)-aziridine-1,2-dicarboxylate (2 g, 8.5021 mmol) and cyclopropanol (1.8340 g, 2 mL, 31.578 mmol) in anhydrous DCM (20 mL) was added boron trifluoride diethyl etherate (115.00 mg, 0.1 mL, 0.8103 mmol) at 0° C. The reaction was stirred at rt overnight. The reaction mixture was diluted with water (50 mL) and DCM (50 mL). Two layers were separated, and the aqueous layer was extracted with DCM (2×50 mL). The combined organic layers were dried over anhydrous sodium sulfate and concentrated under vacuum. The residue was purified by silica gel chromatography using 0 to 20% acetone in hexane to furnish methyl (2R)-2-(benzyloxycarbonylamino)-3-(cyclopropoxy)propanoate (1.578 g, 63%) as a clear liquid. ESI-MS m/z calc. 293.1263, found 294.4 (M+1)⁺; LC method E.

### Step 3: Benzyl N-[(1S)-1-(cyclopropoxymethyl)-2-hydroxy-ethyl]carbamate

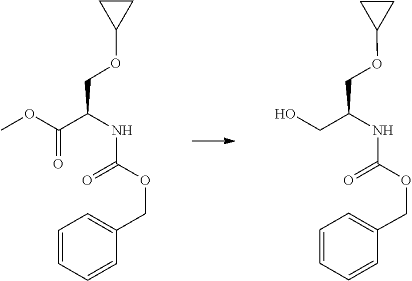 [SMILES: COC(=O)[C@@H](COC1CC1)NC(=O)OCc1ccccc1 ; O=C(N[C@@H](CO)COC1CC1)OCc1ccccc1]

To a solution of methyl (2R)-2-(benzyloxycarbonylamino)-3-(cyclopropoxy)propanoate (1.578 g, 5.3799 mmol) in a solvent mixture of THF (15 mL) and methanol (5 mL) was added sodium borohydride (520 mg, 13.745 mmol). The reaction was stirred at rt for 3 hours. The residue was diluted with ethyl acetate (100 mL) and washed with brine (30 mL), dried over anhydrous sodium sulfate and concentrated under vacuum. The residue was purified by silica gel chromatography using 0 to 30% acetone in hexane to furnish benzyl N-[(1S)-1-(cyclopropoxymethyl)-2-hydroxy-ethyl]carbamate (1.138 g, 80%) as a white solid ESI-MS m/z calc. 265.1314, found 266.2 (M+1)⁺; Retention time: 2.19 minutes; LC method E.

### Step 4: (2S)-2-Amino-3-(cyclopropoxy)propan-1-ol

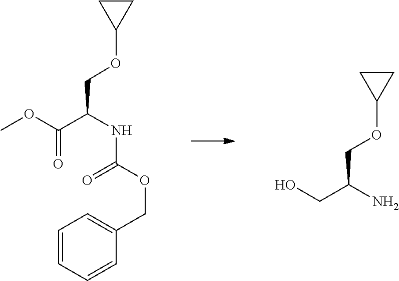 [SMILES: COC(=O)[C@@H](COC1CC1)NC(=O)OCc1ccccc1 ; N[C@@H](CO)COC1CC1]

To a solution of benzyl N-[(1S)-1-(cyclopropoxymethyl)-2-hydroxy-ethyl]carbamate (1.138 g, 4.2894 mmol) in ethyl acetate (20 mL) was added 10% Pd/C (458 mg, 10% w/w, 0.4304 mmol). The reaction was hydrogenated under 1 atm of hydrogen gas for 3 hours. The catalyst was removed by filtration through a pad of Celite. The filtrate was concentrated under vacuum to furnish (2S)-2-amino-3-(cyclopropoxy)propan-1-ol (533 mg, 95%) as a clear gel. ¹H NMR (400 MHz, Acetone-d6) δ 3.61 (dd, J=10.8, 4.5 Hz, 1H), 3.54 (dd, J=9.5, 4.8 Hz, 1H), 3.51-3.43 (m, 2H), 3.30 (tt, J=6.0, 3.0 Hz, 1H), 3.06 (tt, J=6.2, 4.6 Hz, 1H), 1.83 (s, 3H), 0.62-0.53 (m, 2H), 0.53-0.44 (m, 2H).

### Step 5: 3-[[4-[(2R)-2-Amino-3-(cyclopropoxy)propoxy]-6-(2,6-dimethylphenyl)pyrimidin-2-yl]sulfamoyl]benzoic acid

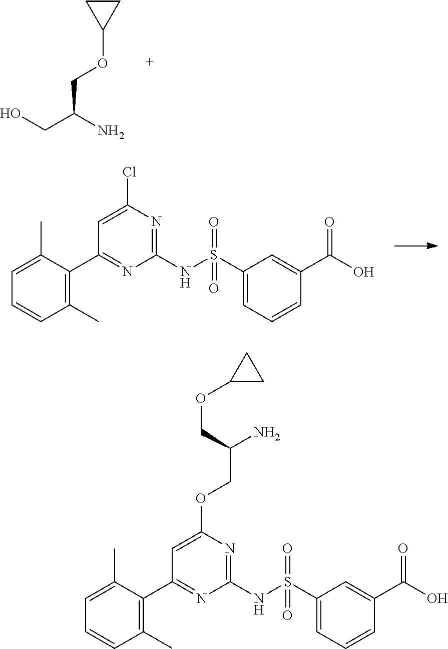

To a solution of 3-[[4-chloro-6-(2,6-dimethylphenyl)pyrimidin-2-yl]sulfamoyl]benzoic acid (1.615 g, 3.8649 mmol) and (2S)-2-amino-3-(cyclopropoxy)propan-1-ol (533 mg, 3.8602 mmol) in anhydrous THE (12 mL) was added tBuONa (2.56 g, 26.638 mmol) at rt. The reaction was stirred at rt for 1 hour. The reaction was quenched with 1 N HCl (35 mL). The product was extracted with ethyl acetate (3×35 mL). The combined organic layers were washed with brine (30 mL), dried over anhydrous sodium sulfate and concentrated under vacuum. The residue was triturated with 1:1 ethyl acetate and hexane (20 mL) to furnish 3-[[4-[(2R)-2-amino-3-(cyclopropoxy)propoxy]-6-(2,6-dimethylphenyl)pyrimidin-2-yl]sulfamoyl]benzoic acid (hydrochloride salt) (1.654 g, 70%) as an off white solid. ¹H NMR (400 MHz, DMSO-d6) δ 13.38 (s, 1H), 8.43 (t, J=1.8 Hz, 1H), 8.26 (s, 3H), 8.19-8.08 (m, 2H), 7.70 (t, J=7.8 Hz, 1H), 7.31-7.19 (m, 1H), 7.13 (d, J=7.7 Hz, 2H), 6.32 (s, 1H), 4.41-4.27 (m, 2H), 3.78-3.70 (m, 2H), 3.70-3.62 (m, 1H), 3.45-3.39 (m, 1H), 1.99 (s, 6H), 0.61-0.54 (m, 2H), 0.53-0.43 (m, 2H). ESI-MS m/z calc. 512.173, found 513.4 (M+1)⁺; Retention time: 1.54 minutes; LC method H.

### Example 24: Preparation of 3-[[4-chloro-6-(2,6-dimethylphenyl)-5-methoxy-pyrimidin-2-yl]sulfamoyl]benzoic acid

### Step 1: tert-Butyl N-tert-butoxycarbonyl-N-(4,6-dichloro-5-methoxy-pyrimidin-2-yl)carbamate

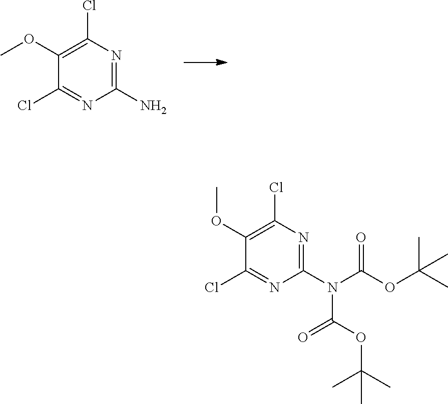 [SMILES: COc1c(Cl)nc(N)nc1Cl ; COc1c(Cl)nc(N(C(=O)OC(C)(C)C)C(=O)OC(C)(C)C)nc1Cl]

To a solution of 4,6-dichloro-5-methoxy-pyrimidin-2-amine (11.73 g, 60.458 mmol) in DCM (200 mL) was added DMAP (591 mg, 4.8376 mmol) and Boc₂O (27.7 g, 29.158 mL, 126.92 mmol). The reaction was stirred for 3 hours at room temperature before being washed with brine (200 mL) and water (200 mL). The organic layer was concentrated in vacuum and dried over sodium sulfate to give tert-butyl N-tert-butoxycarbonyl-N-(4,6-dichloro-5-methoxy-pyrimidin-2-yl)carbamate (21.55 g, 90%). ESI-MS m/z calc. 393.08582, found 394.0 (M+1)⁺; Retention time: 3.44 minutes; LC method E.

### Step 2: tert-Butyl N-tert-butoxycarbonyl-N-[4-chloro-6-(2,6-dimethylphenyl)-5-methoxy-pyrimidin-2-yl]carbamate

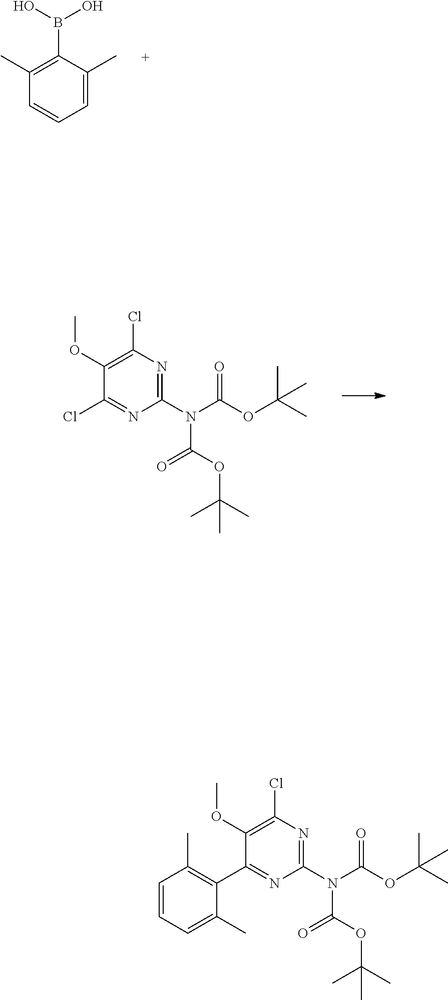

To a solution of tert-butyl N-tert-butoxycarbonyl-N-(4,6-dichloro-5-methoxy-pyrimidin-2-yl)carbamate (31.58 g, 80.101 mmol) dissolved in DME (225 mL) and water (31 mL) was added (2,6-dimethylphenyl)boronic acid (16.5 g, 110.01 mmol) and cesium carbonate (68 g, 208.71 mmol) at room temperature. The solution was stirred for 10 min while being bubbled with a nitrogen stream. Then Pd(dppf)Cl₂ (5.86 g, 8.0087 mmol) was added to the solution and heated to 80° C. overnight. The solution was cooled to room temperature before being diluted with water (250 mL) and extracted with ethyl acetate (2×300 mL). The combined organic layer was washed with brine (400 mL) and dried over sodium sulfate before being concentrated under vacuum. The organic residue was purified by silica gel chromatography eluting 0-60% ethyl acetate-hexanes to give tert-butyl N-tert-butoxycarbonyl-N-[4-chloro-6-(2,6-dimethylphenyl)-5-methoxy-pyrimidin-2-yl]carbamate (50.35 g, 135%). ESI-MS m/z calc. 463.1874, found 464.2 (M+1)⁺; Retention time: 3.68 minutes; LC method E.

### Step 3: 4-Chloro-6-(2,6-dimethylphenyl)-5-methoxy-pyrimidin-2-amine

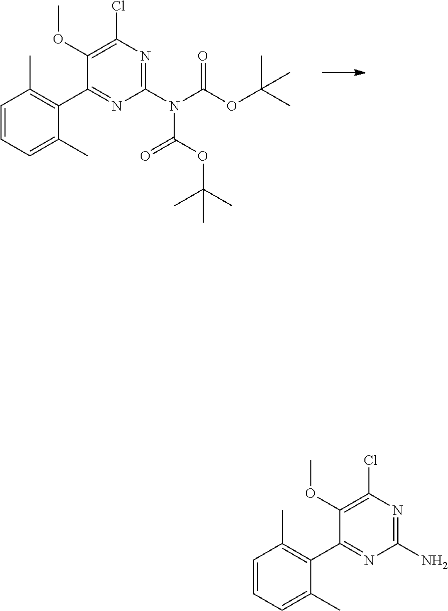 [SMILES: COc1c(Cl)nc(N(C(=O)OC(C)(C)C)C(=O)OC(C)(C)C)nc1-c1c(C)cccc1C ; COc1c(Cl)nc(N)nc1-c1c(C)cccc1C]

To a solution of tert-butyl N-tert-butoxycarbonyl-N-[4-chloro-6-(2,6-dimethylphenyl)-5-methoxy-pyrimidin-2-yl]carbamate (50.35 g, 108.52 mmol) in DCM (500 mL) was added HCl (100 mL of 4 M, 400.00 mmol) in dioxane. The solution was stirred at room temperature overnight before being concentrated in vacuum. The residue was then basified with sodium bicarbonate (400 mL) and extracted with ethyl acetate (500 mL). The organic layer was washed with brine (500 mL) and dried over sodium sulfate. The organic phase was concentrated then triturated with hexanes (2×50 mL) to give 4-chloro-6-(2,6-dimethylphenyl)-5-methoxy-pyrimidin-2-amine (10.16 g, 36%). ESI-MS m/z calc. 263.08255, found 264.1 (M+1)⁺; Retention time: 2.73 minutes; LC method E.

### Step 4: Methyl 3-[[4-chloro-6-(2,6-dimethylphenyl)-5-methoxy-pyrimidin-2-yl]sulfamoyl]benzoate

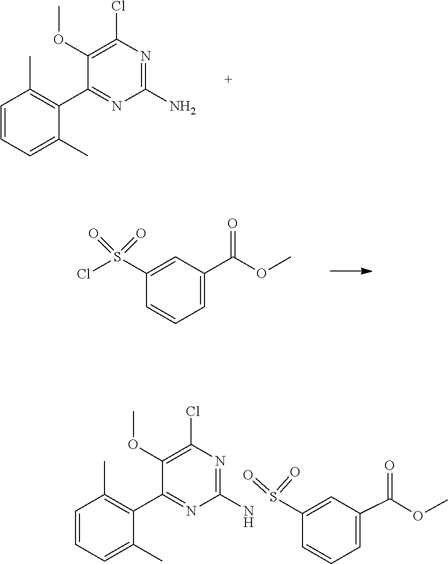

To a solution of 4-chloro-6-(2,6-dimethylphenyl)-5-methoxy-pyrimidin-2-amine (223 mg, 0.8456 mmol) in THE (6 mL) at 0° C. was added methyl 3-chlorosulfonylbenzoate (496 mg, 2.1137 mmol). Then Lithium tert-amoxide (584.00 mg, 2 mL of 40% w/w, 2.4830 mmol) was added to the solution dropwise keeping the temperature below 5° C. The solution was allowed to warm to room temperature while it stirred for 3 hours. The solution was acidified with 1 M HCl (5 mL) and extracted with ethyl acetate (20 mL). The organic layer was washed with brine (20 mL) and dried over sodium sulfate. The organic layer was then concentrated in vacuum to give methyl 3-[[4-chloro-6-(2,6-dimethylphenyl)-5-methoxy-pyrimidin-2-yl]sulfamoyl]benzoate (386 mg, 99%) of a yellow solid. ESI-MS m/z calc. 461.0812, found 462.1 (M+1)⁺; Retention time: 3.18 minutes; LC method E.

### Step 5: 3-[[4-Chloro-6-(2,6-dimethylphenyl)-5-methoxy-pyrimidin-2-yl]sulfamoyl]benzoic acid

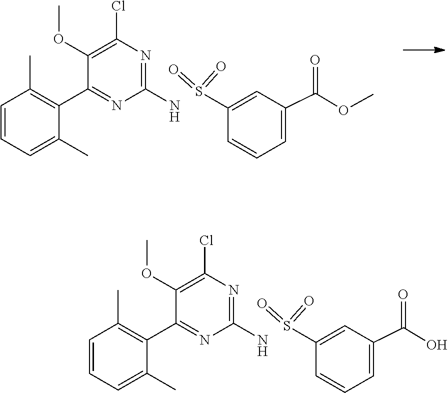

To a solution of methyl 3-[[4-chloro-6-(2,6-dimethylphenyl)-5-methoxy-pyrimidin-2-yl]sulfamoyl]benzoate (386 mg, 0.8356 mmol) in THE (10 mL) was added an aqueous solution of NaOH (5 mL of 1 M, 5.0000 mmol) and stirred for 1 hour at room temperature. The solution was acidified using 1 M HCl (5 mL) and extracted with ethyl acetate (2×20 mL) before being washed with brine (20 mL). The organic layer was dried over sodium sulfate and concentrated in vacuum to give 3-[[4-chloro-6-(2,6-dimethylphenyl)-5-methoxy-pyrimidin-2-yl]sulfamoyl]benzoic acid (314 mg, 84%) as a white solid. ESI-MS m/z calc. 447.06558, found 448.1 (M+1)⁺; Retention time: 2.9 minutes; LC method E.

### Example 25: Preparation of 3-[[4-[(3S)-3-amino-5,5-dimethyl-hexyl]-6-(2,6-dimethylphenyl)-5-methoxy-pyrimidin-2-yl]sulfamoyl]benzoic acid

### Step 1: 3-[[4-[(3R)-3-(tert-Butoxycarbonylamino)-5,5-dimethyl-hex-1-ynyl]-6-(2,6-dimethylphenyl)-5-methoxy-pyrimidin-2-yl]sulfamoyl]benzoic acid

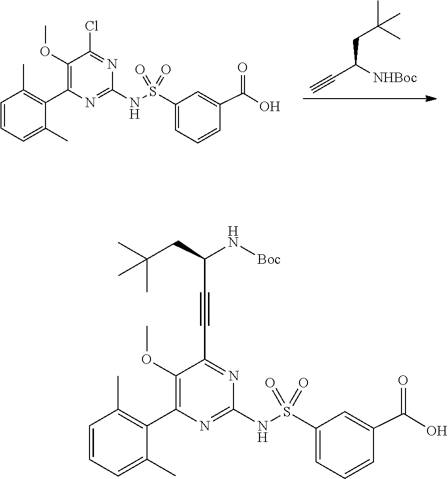 [SMILES: COc1c(Cl)nc(NS(=O)(=O)c2cccc(C(=O)O)c2)nc1-c1c(C)cccc1C ; C#C[C@@H](CC(C)(C)C)NC(=O)OC(C)(C)C ; COc1c(C#C[C@@H](CC(C)(C)C)NC(=O)OC(C)(C)C)nc(NS(=O)(=O)c2cccc(C(=O)O)c2)nc1-c1c(C)cccc1C]

A flask equipped with a reflux condenser was charged with 3-[[4-chloro-6-(2,6-dimethylphenyl)-5-methoxy-pyrimidin-2-yl]sulfamoyl]benzoic acid (3.5 g, 7.8144 mmol), and tert-butyl N-[(1R)-1-ethynyl-3,3-dimethyl-butyl]carbamate (2.81 g, 10.916 mmol) and purged with argon. Then solids were treated with DMSO (38 mL) and degassed an additional 4 h with argon. Then the solution was treated with Pd(PPh₃)₄ (994 mg, 0.8602 mmol) and CuI (195 mg, 1.0239 mmol). Then argon was bubbled through the reaction solution for 60 minutes. The reaction was treated with TEA (7.9860 g, 11 mL, 78.921 mmol) at RT, heated in an oil bath at 75° C. for 5 h and 15 min, and cooled to rt for 12 h. Then the reaction was treated with more tert-butyl N-[(1R)-1-ethynyl-3-methyl-butyl]carbamate (0.94 g, 4.4486 mmol) and sparged with argon for 60 minutes. Pd(PPh₃)₄ (271 mg, 0.2345 mmol), CuI (30 mg, 0.1575 mmol) were added and then sparged with argon for 60 min and then treated with TEA (3.6300 g, 5 mL, 35.873 mmol). The reaction was heated an additional 5 h at 75° C. The reaction mixture was cooled to rt and poured into water (375 mL) and diluted with Et₂O (250 mL). The two layers were separated. The ether layer was back extracted with 1M NaOH (10 mL). The ether layer was discarded. The combined aqueous phase was acidified with aqueous 10% w/v citric acid to until pH=4 and extracted with ethyl acetate (3×100 mL). The combined organic layers were washed with brine (3×125 mL), dried over anhydrous sodium sulfate and concentrated under vacuum to give a brown crude mixture (7 g). The crude residue was purified by flash chromatography (120 g SiO₂, dry loaded, eluted with 0 to 35% acetone in hexane (buffered with 0.3% acetic acid) over a 30 min. gradient). The appropriate fractions were combined and concentrated in vacuo. The residue was diluted with Et₂O and concentrated in vacuo, this cycle was repeated 3 times to obtain the target, 3-[[4-[(3R)-3-(tert-butoxycarbonylamino)-5,5-dimethyl-hex-1-ynyl]-6-(2,6-dimethylphenyl)-5-methoxy-pyrimidin-2-yl]sulfamoyl]benzoic acid (2.87 g, 55%), as a brittle yellow solid. ¹H NMR (500 MHz, DMSO-d6) δ 13.36 (s, 1H), 11.93 (s, 1H), 8.36 (s, 1H), 8.15 (d, J=7.8 Hz, 1H), 8.10 (d, J=8.0 Hz, 1H), 7.63 (t, J=7.8 Hz, 1H), 7.55 (d, J=8.6 Hz, 1H), 7.23 (t, J=7.6 Hz, 1H), 7.08 (d, J=7.6 Hz, 2H), 4.59 (q, J=7.2 Hz, 1H), 3.45 (s, 3H), 1.78 (s, 6H), 1.75-1.64 (m, 2H), 1.39 (s, 9H), 0.95 (s, 9H). ESI-MS m/z calc. 636.2618, found 637.6 (M+1)⁺; Retention time: 6.63 minutes; LC method D.

### Step 2: 3-[[4-[(3S)-3-(tert-Butoxycarbonylamino)-5,5-dimethyl-hexyl]-6-(2,6-dimethylphenyl)-5-methoxy-pyrimidin-2-yl]sulfamoyl]benzoic acid

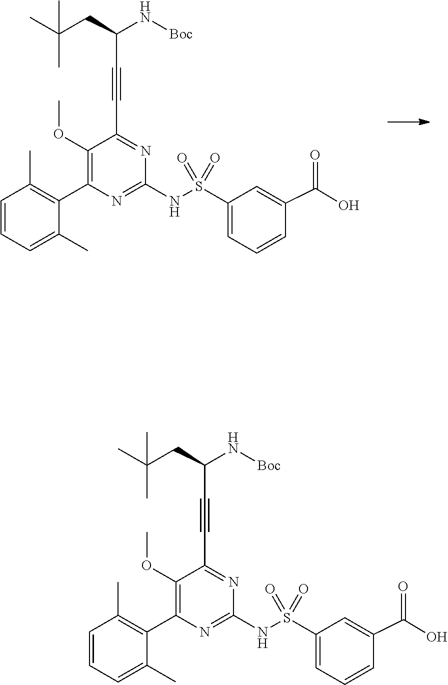 [SMILES: COc1c(C#C[C@@H](CC(C)(C)C)NC(=O)OC(C)(C)C)nc(NS(=O)(=O)c2cccc(C(=O)O)c2)nc1-c1c(C)cccc1C ; COc1c(C#C[C@@H](CC(C)(C)C)NC(=O)OC(C)(C)C)nc(NS(=O)(=O)c2cccc(C(=O)O)c2)nc1-c1c(C)cccc1C]

A solution of 3-[[4-[(3R)-3-(tert-butoxycarbonylamino)-5,5-dimethyl-hex-1-ynyl]-6-(2,6-dimethylphenyl)-5-methoxy-pyrimidin-2-yl]sulfamoyl]benzoic acid (2.29 g, 3.4165 mmol) in AcOH (35 mL) was charged with Pd/C (800 mg, 10% w/w, 0.7517 mmol) and sparged with N₂ for 6 times to a pressure of 40 psi. Then the vessel was charged with H₂ (60 PSI) 6 times and held at 60 PSI. The reaction was stirred at room temperature for 20 h in a Parr shaker. The reaction was treated with additional Pd/C (800 mg, 10% w/w, 0.7517 mmol) and charged with H₂ g (60 psi) in the same fashion. The pressure was reduced H₂ g (20 psi). The reaction was stirred at room temperature for 20 h in a Parr shaker. The pressure was increased to (60 PSI) H₂. The reaction was stirred at room temperature for 20 h. The reaction was filtered through celite and washed with MeOH (500 mL). The reaction was concentrated in vacuo to give an orange solid. The material was again dissolved in AcOH (35 mL), treated with Pd/C (800 mg, 10% w/w, 0.7517 mmol) sparged with N₂ for 6 times to a pressure of 40 psi. Then the vessel was charged with H₂ (60 PSI) 6 times and held at 60 PSI. The reaction was stirred at room temperature for 33 h in a Parr shaker. The reaction was filtered through celite and washed with MeOH (500 mL). The filtrates were collected and concentrated under reduced pressure to obtain the crude mixture of the target, 3-[[4-[(3S)-3-(tert-butoxycarbonylamino)-5,5-dimethyl-hexyl]-6-(2,6-dimethylphenyl)-5-methoxy-pyrimidin-2-yl]sulfamoyl]benzoic acid (2.37 g, 97%), as an orange solid. ¹H NMR (500 MHz, Chloroform-d) δ 9.17-9.01 (m, 1H), 8.32-8.15 (m, 2H), 7.58 (t, J=7.9, 7.9 Hz, 1H), 7.23 (t, J=7.6, 7.6 Hz, 1H), 7.09 (t, J=7.3, 7.3 Hz, 2H), 4.54 (d, J=9.8 Hz, 1H), 3.91-3.63 (m, 1H), 3.28 (s, 3H), 2.93-2.67 (m, 2H), 2.04 (s, 3H), 1.99 (s, 3H), 1.89-1.68 (m, 2H), 1.52-1.43 (m, 9H), 1.41-1.23 (m, 2H), 0.99-0.93 (m, 9H). ESI-MS m/z calc. 640.2931, found 641.5 (M+1)⁺; Retention time: 6.61 minutes; LC method D.

### Step 3: 3-[[4-[(3S)-3-Amino-5,5-dimethyl-hexyl]-6-(2,6-dimethylphenyl)-5-methoxy-pyrimidin-2-yl]sulfamoyl]benzoic acid

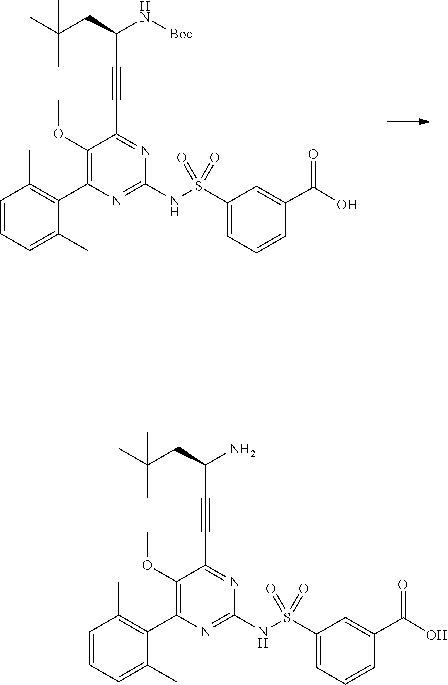 [SMILES: COc1c(C#C[C@@H](CC(C)(C)C)NC(=O)OC(C)(C)C)nc(NS(=O)(=O)c2cccc(C(=O)O)c2)nc1-c1c(C)cccc1C ; COc1c(C#C[C@H](N)CC(C)(C)C)nc(NS(=O)(=O)c2cccc(C(=O)O)c2)nc1-c1c(C)cccc1C]

A flask charged with 3-[[4-[(3S)-3-(tert-butoxycarbonylamino)-5,5-dimethyl-hexyl]-6-(2,6-dimethylphenyl)-5-methoxy-pyrimidin-2-yl]sulfamoyl]benzoic acid (2.37 g, 3.2991 mmol) was purged with argon. The solids were treated with TFA (9.5 mL) at RT. The reaction was stirred at room temperature for 30 minutes. The solvent was removed in vacuo to give the target compound as a dark orange viscous residue (3.21 g). The residue was dissolved into TBME (9 mL) and added dropwise into n-hexanes (200 mL) and the precipitate was rapidly collected by filtration and dried in vacuo to give the target, 3-[[4-[(3S)-3-amino-5,5-dimethyl-hexyl]-6-(2,6-dimethylphenyl)-5-methoxy-pyrimidin-2-yl]sulfamoyl]benzoic acid (Trifluoroacetic Acid (1)) (2.1938 g, 97%), as a light yellow orange powder. ¹H NMR (500 MHz, DMSO-d6) δ 13.46 (s, 1H), 11.79 (s, 1H), 8.45 (t, J=1.8 Hz, 1H), 8.17 (dt, J=7.8, 1.4 Hz, 1H), 8.13 (dt, J=8.0, 1.5 Hz, 1H), 7.78 (s, 3H), 7.67 (t, J=7.8 Hz, 1H), 7.25 (t, J=7.6 Hz, 1H), 7.11 (d, J=7.6 Hz, 2H), 3.28-3.21 (m, 4H), 2.93-2.76 (m, 2H), 1.94-1.79 (m, 8H), 1.53-1.42 (m, 2H), 0.90 (s, 9H). ESI-MS m/z calc. 540.24066, found 541.3 (M+1)⁺; Retention time: 2.01 minutes; LC method H.

### Example 26: Preparation of 3-[[4-[(3S)-3-amino-5,5-dimethyl-hexyl]-6-(2,6-dimethylphenyl)pyrimidin-2-yl]sulfamoyl]benzoic acid

### Step 1: tert-Butyl N-[(1R)-1-[methoxy(methyl)carbamoyl]-3,3-dimethyl-butyl]carbamate

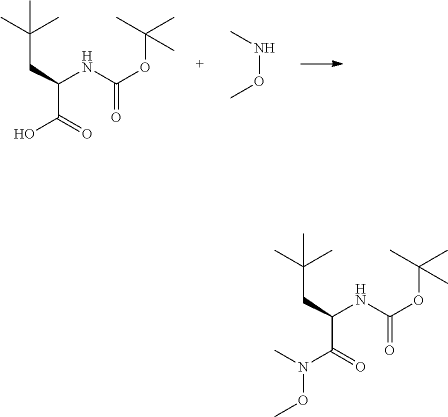

A solution of (2R)-2-(tert-butoxycarbonylamino)-4,4-dimethyl-pentanoic acid (29.6 g, 120.66 mmol) in DCM (450 mL) and stirred at −10° C. Then HOBt (17.3 g, 128.03 mmol) and 1-(3-dimethylaminopropyl)-3-ethylcarbodiimide (hydrochloride salt) (34.6 g, 180.49 mmol) were added. The reaction was stirred for 15 minutes at −10° C. and N-methoxymethanamine (hydrochloride salt) (12.15 g, 124.56 mmol) was added in one portion followed by dropwise addition of DIPEA (38.955 g, 52.5 mL, 301.41 mmol) over 15 min. (internal temp. −9° C.). The bath was −15° C. The reaction was warmed to rt over 16 hours. The reaction treated with was quenched with HCl (270 mL of 1 M, 270.00 mmol) until pH=3. The mixture was filtered, and the two layers of filtrate were separated. The organic layer was washed with saturated sodium bicarbonate (500 mL), brine (100 mL), dried over anhydrous magnesium sulfate and concentrated under vacuum to furnish tert-butyl N-[(1R)-1-[methoxy(methyl)carbamoyl]-3,3-dimethyl-butyl]carbamate (33.52 g, 87%) as a colorless wax. ¹H NMR (500 MHz, Chloroform-d) δ 5.10-4.92 (m, 1H), 4.85-4.69 (m, 1H), 3.79 (s, 3H), 3.19 (s, 3H), 1.56 (dd, J=14.3, 3.0 Hz, 2H), 1.42 (s, 9H), 0.97 (s, 9H). ESI-MS m/z calc. 288.2049, found 289.3 (M+1)⁺; Retention time: 4.95 minutes; LC method D.

### Step 2: tert-Butyl N-[(1R)-1-ethynyl-3,3-dimethyl-butyl]carbamate

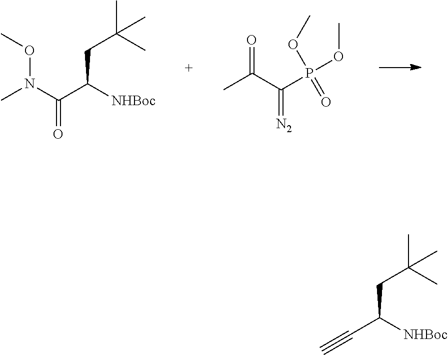

A solution of tert-butyl N-[(1R)-1-[methoxy(methyl)carbamoyl]-3,3-dimethyl-butyl]carbamate (21.25 g, 73.687 mmol) in anhydrous DCM (550.00 mL) was treated with DIBAL-H (184 mL of 1 M, 184.00 mmol) in toluene dropwise over 80 min. at −72° C. (internal temperature). The reaction was stirred at the same temperature for 2 h. Excess of DIBAL-H was quenched with MeOH (400 mL), over 70 minutes. The reaction was slowly raised to 0° C. over 70 minutes. Then the solution was treated with potassium carbonate (30.5 g, 220.69 mmol) in one portion and a solution of 1-dimethoxyphosphoryl-1-(imino-λ⁴-azanylidene)propan-2-one (29.440 g, 23 mL, 152.45 mmol) in MeOH (400 mL) was added to the reaction (0° C. internal) over 2 h and 30 minutes. The bath was warmed slowly to room temperature over 18 h. The reaction was quenched with saturated aqueous Rochells's salt (700 mL) and the reaction was stirred for 60 minutes. The majority of the DCM and MeOH was removed in vacuo and the reaction mixture was extracted with EtOAc (400 mL). The aqueous layer was extracted with EtOAc (3×150 mL). The combined organic layers were washed with brine (2×100 mL), dried over anhydrous sodium sulfate and concentrated under vacuum gave a yellow liquid crude product. The crude residue was purified by flash chromatography (Combiflash 330 g SiO₂, liquid loaded, eluted with 0-20% Et₂O in hexanes over a 10 column volume gradient). The pure fractions were combined and the solvent was evaporated in vacuo to obtain the product, tert-butyl N-[(1R)-1-ethynyl-3,3-dimethyl-butyl]carbamate (9.3 g, 50%), as a clear liquid. ¹H NMR (500 MHz, Chloroform-d) δ 4.64 (m, 1H), 4.47 (m, 1H), 2.27 (d, J=2.3 Hz, 1H), 1.72-1.65 (m, 1H), 1.57 (dd, J=13.8, 6.3 Hz, 1H), 1.44 (s, 9H), 0.98 (s, 9H). ESI-MS m/z calc. 225.17288, found 226.5 (M+1)⁺; Retention time: 5.43 minutes; LC method D.

### Step 3: 3-[[4-[(3R)-3-(tert-Butoxycarbonylamino)-5,5-dimethyl-hex-1-ynyl]-6-(2,6-dimethylphenyl)pyrimidin-2-yl]sulfamoyl]benzoic acid

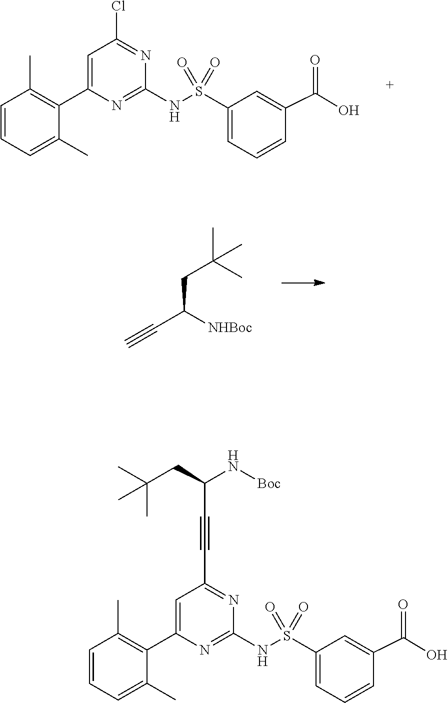 [SMILES: Cc1cccc(C)c1-c1cc(Cl)nc(NS(=O)(=O)c2cccc(C(=O)O)c2)n1 ; C#C[C@@H](CC(C)(C)C)NC(=O)OC(C)(C)C ; Cc1cccc(C)c1-c1cc(C#C[C@@H](CC(C)(C)C)NC(=O)OC(C)(C)C)nc(NS(=O)(=O)c2cccc(C(=O)O)c2)n1]

A flask was charged with 3-[[4-chloro-6-(2,6-dimethylphenyl)pyrimidin-2-yl]sulfamoyl]benzoic acid (11.5 g, 27.521 mmol), and tert-butyl N-[(1R)-1-ethynyl-3,3-dimethyl-butyl]carbamate (7.86 g, 34.883 mmol) and purged with argon for 2 h, then the solids were treated with DMSO (115 mL) and degassed an additional 2 h. Then the solution was treated with, Pd(PPh₃)₄ (1.59 g, 1.3760 mmol) and CuI (522 mg, 2.7409 mmol) and the reaction sparged with argon for an additional 1 h and 30 minutes. Then the solution was treated with TEA (23.232 g, 32 mL, 229.59 mmol), heated in an oil bath at 80° C. for 4 h and 30 minutes. Then the reaction was treated with tert-butyl N-[(1R)-1-ethynyl-3-methyl-butyl]carbamate (1.1 g, 5.2058 mmol) in DMSO (4 mL) at 80° C. and heated at this temperature for an additional 45 min., and then cooled to RT. The reaction mixture was diluted with water (1.2 L) and diethyl ether (800 mL) and washed an additional time with Et₂O (400 mL). The two layers were separated and the ether layer was set aside. The aqueous phase was diluted with EtOAC (400 mL) and acidified to pH 4 with aqueous citric acid (10% wt/vol). The layers were separated and then aqueous phase was extracted with ethyl acetate (3×250 mL). The combined organic layers were washed with brine (4×100 mL), dried over anhydrous sodium sulfate and concentrated under vacuum. The crude residue was purified by flash chromatography (Combiflash 330 g SiO₂, dry loaded, eluted with 20-40% acetone in hexanes (buffered with 0.3% acetic acid) over a 10 column volume gradient). The appropriate fractions were combined and concentrated in vacuo. The residue was triturated with hexane (500 mL) to furnish 3-[[4-[(3R)-3-(tert-butoxycarbonylamino)-5,5-dimethyl-hex-1-ynyl]-6-(2,6-dimethylphenyl)pyrimidin-2-yl]sulfamoyl]benzoic acid (11 g, 64%) as a yellow solid. ¹H NMR (500 MHz, Chloroform-d) δ 9.12 (m, 1H), 8.27 (m, 2H), 7.55 (t, J=7.8 Hz, 1H), 7.21 (t, J=7.6 Hz, 1H), 7.08 (d, J=7.6 Hz, 2H), 6.86 (s, 1H), 4.95 (d, J=9.1 Hz, 1H), 4.85-4.75 (m, 1H), 1.97 (s, 6H), 1.81 (m, 1H), 1.68 (m, 1H), 1.53-1.45 (m, 9H), 1.02 (s, 9H). ESI-MS m/z calc. 606.2512, found 607.3 (M+1)⁺; Retention time: 6.73 minutes; LC method D.

### Step 4: 3-[[4-[(3S)-3-(tert-Butoxycarbonylamino)-5,5-dimethyl-hexyl]-6-(2,6-dimethylphenyl)pyrimidin-2-yl]sulfamoyl]benzoic acid

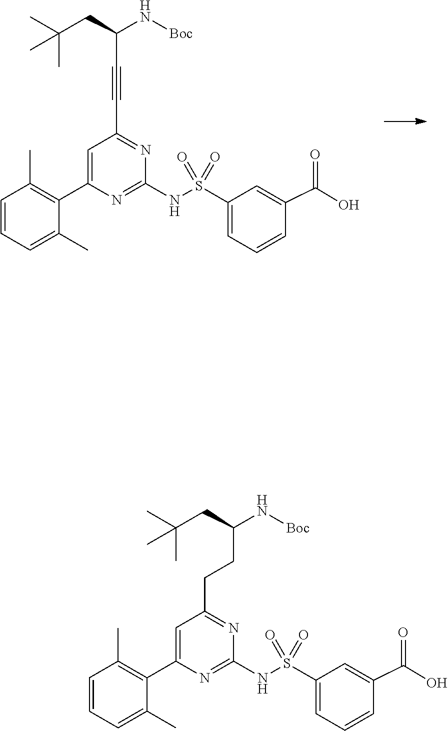 [SMILES: Cc1cccc(C)c1-c1cc(C#C[C@@H](CC(C)(C)C)NC(=O)OC(C)(C)C)nc(NS(=O)(=O)c2cccc(C(=O)O)c2)n1 ; Cc1cccc(C)c1-c1cc(CC[C@@H](CC(C)(C)C)NC(=O)OC(C)(C)C)nc(NS(=O)(=O)c2cccc(C(=O)O)c2)n1]

A solution of 3-[[4-[(3R)-3-(tert-butoxycarbonylamino)-5,5-dimethyl-hex-1-ynyl]-6-(2,6-dimethylphenyl)pyrimidin-2-yl]sulfamoyl]benzoic acid (11 g, 17.586 mmol) in ethanol (380 mL) was treated with 10% Pd/C (2.75 g, 2.5841 mmol) on carbon. The reaction was purged with nitrogen, then H₂ gas was bubbled through the suspension and the reaction was stirred at room temperature under 1 atm of hydrogen for 3 hour. The catalyst was removed by filtration. The filtrate was concentrated under vacuum to furnish 3-[[4-[(3S)-3-(tert-butoxycarbonylamino)-5,5-dimethyl-hexyl]-6-(2,6-dimethylphenyl)pyrimidin-2-yl]sulfamoyl]benzoic acid (10.71 g, 99%) as a yellow solid. ¹H NMR (500 MHz, Chloroform-d) δ 9.13 (s, 1H), 8.30-8.16 (m, 2H), 7.57 (t, J=7.7 Hz, 1H), 7.20 (t, J=7.6 Hz, 1H), 7.07 (d, J=7.6 Hz, 2H), 6.75 (s, 1H), 4.49 (d, J=9.5 Hz, 1H), 3.84-3.76 (m, 1H), 2.89-2.61 (m, 2H), 1.98 (s, 6H), 1.79 (m, 2H), 1.45 (m, 10H), 1.35-1.27 (m, 1H), 0.95 (s, 9H). ESI-MS m/z calc. 610.28253, found 611.5 (M+1)⁺; Retention time: 6.61 minutes; LC method D.

### Step 5: 3-[[4-[(3S)-3-Amino-5,5-dimethyl-hexyl]-6-(2,6-dimethylphenyl)pyrimidin-2-yl]sulfamoyl]benzoic acid

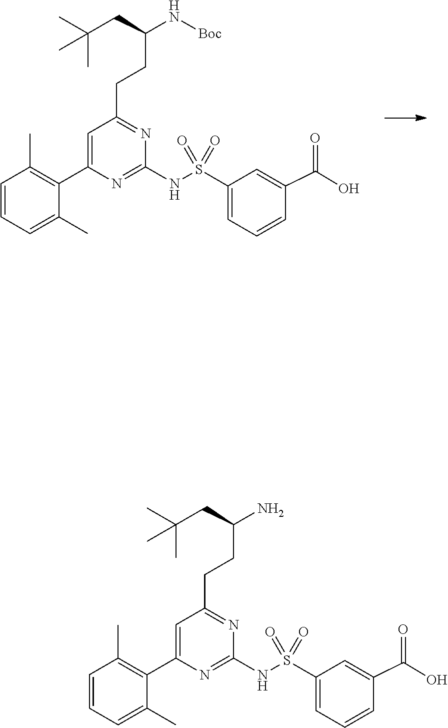 [SMILES: Cc1cccc(C)c1-c1cc(CC[C@@H](CC(C)(C)C)NC(=O)OC(C)(C)C)nc(NS(=O)(=O)c2cccc(C(=O)O)c2)n1 ; Cc1cccc(C)c1-c1cc(CC[C@H](N)CC(C)(C)C)nc(NS(=O)(=O)c2cccc(C(=O)O)c2)n1]

A solution of 3-[[4-[(3S)-3-(tert-butoxycarbonylamino)-5,5-dimethyl-hexyl]-6-(2,6-dimethylphenyl)pyrimidin-2-yl]sulfamoyl]benzoic acid (10.71 g, 17.360 mmol) in DCM (45 mL) was treated with HCl in dioxane (25 mL of 4 M, 100.00 mmol) over 5 min. at RT. The reaction was stirred at rt for 2 h and the solvent was removed under reduced pressure. The residue was triturated with TBME (60 mL), filtered, and rinsed with TBME (30 mL), and the solids were dried in vacuo to obtain the target the target, 3-[[4-[(3S)-3-amino-5,5-dimethyl-hexyl]-6-(2,6-dimethylphenyl)pyrimidin-2-yl]sulfamoyl]benzoic acid (hydrochloride salt) (9.7794 g, 100%), as a light yellow powder. ¹H NMR (500 MHz, DMSO-d6) δ 13.38 (s, 1H), 11.91 (s, 1H), 8.47 (s, 1H), 8.16 (d, J=7.8 Hz, 2H), 8.01 (s, 3H), 7.68 (t, J=7.8 Hz, 1H), 7.22 (t, J=7.6 Hz, 1H), 7.10 (d, J=7.6 Hz, 2H), 7.02 (s, 1H), 3.11-3.04 (m, 1H), 2.89-2.75 (m, 2H), 1.97-1.86 (m, 2H), 1.85 (s, 6H), 1.50-1.39 (m, 2H), 0.81 (s, 9H). ESI-MS m/z calc. 510.23007, found 511.6 (M+1)⁺; Retention time: 2.03 minutes; LC method H.

### Example 27: Preparation of 3-[[4-chloro-6-(2,6-dimethylphenyl)-5-methyl-pyrimidin-2-yl]sulfamoyl]benzoic acid

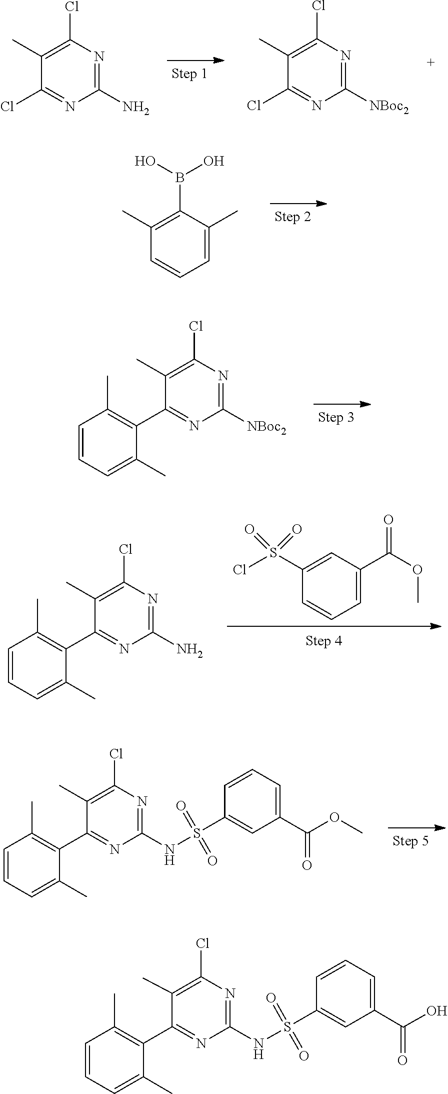

### Step 1: tert-Butyl N-tert-butoxycarbonyl-N-(4,6-dichloro-5-methyl-pyrimidin-2-yl)carbamate

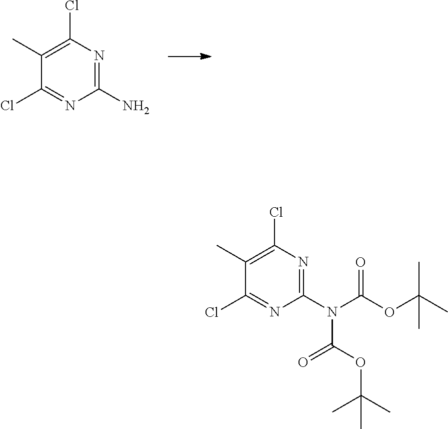

To a solution of 4,6-dichloro-5-methyl-pyrimidin-2-amine (57.85 g, 318.47 mmol) in DCM (580 mL) was added tert-butoxycarbonyl tert-butyl carbonate (159.92 g, 168.34 mL, 710.77 mmol) and DMAP (3.96 g, 32.090 mmol) at RT. The reaction was stirred for 3 h. The reaction mixture was quenched with DI H₂O (250 mL). DCM (100 mL) was added. The layers were separated, and the aqueous layer was extracted with DCM (2×250 mL). The combined organic layers were washed with aqueous saturated NaCl (250 mL), dried over anhydrous sodium sulfate, filtered and concentrated under vacuum. Crude Mass=125.71 g (Yellow Solid). The yellow solid was triturated with hexanes (300 mL, 3 h), filtered through a Type “M” Glass filter by vacuum and the solids were rinsed with Hexanes (2×200 mL). Final Product (101.00 g) was obtained as a yellow solid. tert-butyl N-tert-butoxycarbonyl-N-(4,6-dichloro-5-methyl-pyrimidin-2-yl)carbamate (101.00 g, 80%). ¹H NMR (500 MHz, Chloroform-d) δ 2.48 (s, 3H), 1.47 (s, 18H). ESI-MS m/z calc. 377.0909, found 378.0 (M+1)⁺; Retention time: 3.39 minutes; LC method E.

### Step 2: tert-Butyl N-tert-butoxycarbonyl-N-[4-chloro-6-(2,6-dimethylphenyl)-5-methyl-pyrimidin-2-yl]carbamate

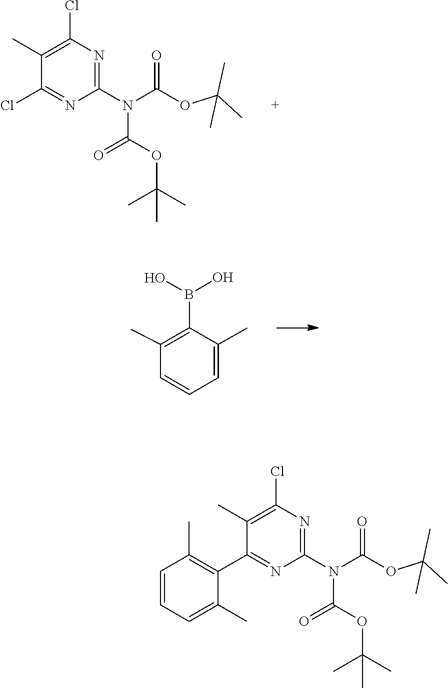

To a solution of tert-butyl N-tert-butoxycarbonyl-N-(4,6-dichloro-5-methyl-pyrimidin-2-yl)carbamate (120.85 g, 319.50 mmol) dissolved in DME (850 mL) and water (120 mL) was added (2,6-dimethylphenyl)boronic acid (57.5 g, 383.38 mmol) and cesium carbonate (271 g, 831.75 mmol) at room temperature. The solution was stirred for 10 min while being bubbled with a nitrogen stream. Then Pd(dppf)Cl₂ (11.7 g, 15.990 mmol) was added to the solution and heated to 80° C. overnight. The solution was cooled to room temperature before being diluted with water (500 mL) and extracted with ethyl acetate (2×1 L). The combined organic layer was washed with brine (1 L) and dried over sodium sulfate before being concentrated in vacuum. The organic residue was filtered through a pad of silica gel and washed with a solution of 1:3 ethyl acetate-hexanes (3×1 L) to give tert-butyl N-tert-butoxycarbonyl-N-[4-chloro-6-(2,6-dimethylphenyl)-5-methyl-pyrimidin-2-yl]carbamate (100.71 g, 58%). ESI-MS m/z calc. 447.19247, found 448.1 (M+1)⁺; Retention time: 4.24 minutes; LC method E.

### Step 3: 4-Chloro-6-(2,6-dimethylphenyl)-5-methyl-pyrimidin-2-amine

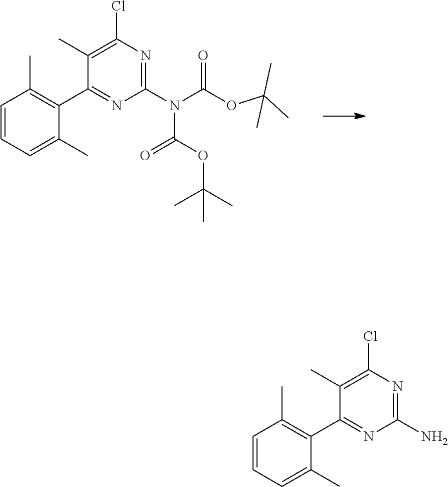 [SMILES: Cc1cccc(C)c1-c1nc(N(C(=O)OC(C)(C)C)C(=O)OC(C)(C)C)nc(Cl)c1C ; Cc1cccc(C)c1-c1nc(N)nc(Cl)c1C]

To a solution of tert-butyl N-tert-butoxycarbonyl-N-[4-chloro-6-(2,6-dimethylphenyl)-5-methyl-pyrimidin-2-yl]carbamate (100.71 g, 224.82 mmol) in DCM (500 mL) was added HCl (200 mL of 4 M, 800.00 mmol) in dioxane. The solution was stirred at room temperature overnight before being concentrated in vacuum. The residue was then basified with sodium bicarbonate (500 mL) and extracted with ethyl acetate (1 L). The organic layer was washed with brine (400 mL) and dried over sodium sulfate. The organic phase was concentrated then the residue was triturated with hexanes (2×200 mL) to give 4-chloro-6-(2,6-dimethylphenyl)-5-methyl-pyrimidin-2-amine (54.88 g, 99%) as an off-white solid. ESI-MS m z calc. 247.08763, found 248.2 (M+1)⁺; Retention time: 2.94 minutes; LC method E.

### Step 4: Methyl 3-[[4-chloro-6-(2,6-dimethylphenyl)-5-methyl-pyrimidin-2-yl]sulfamoyl]benzoate

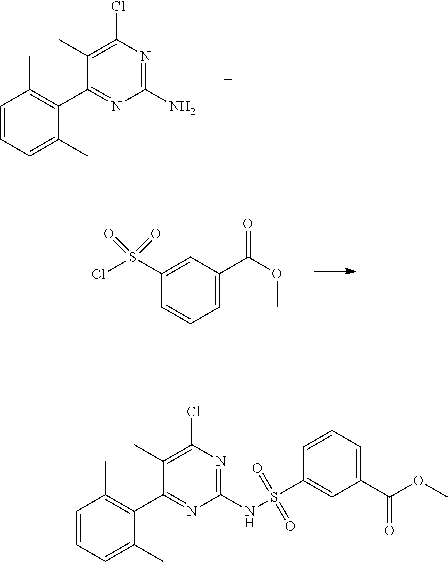 [SMILES: Cc1cccc(C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3C)n2)c1]

To a solution of 4-chloro-6-(2,6-dimethylphenyl)-5-methyl-pyrimidin-2-amine (35 g, 141.29 mmol) in THE (400 mL) at 0° C. was added methyl 3-chlorosulfonylbenzoate (50 g, 213.08 mmol). Then Lithium tert-amoxide (46.428 g, 159 mL of 40% w/w, 197.40 mmol) was added to the solution dropwise keeping the temperature below 5° C. The solution was allowed to warm to room temperature while it stirred for 3 hours. The solution was acidified with 1 M HCl (200 mL) and extracted with ethyl acetate (3×200 mL). The organic layer was washed with brine (300 mL) and dried over sodium sulfate. The organic layer was then concentrated in vacuum to give methyl 3-[[4-chloro-6-(2,6-dimethylphenyl)-5-methyl-pyrimidin-2-yl]sulfamoyl]benzoate (63.01 g, 100%) as a yellow solid. ESI-MS m/z calc. 445.0863, found 446.2 (M+1)⁺; Retention time: 3.63 minutes; LC method E.

### Step 5: 3-[[4-Chloro-6-(2,6-dimethylphenyl)-5-methyl-pyrimidin-2-yl]sulfamoyl]benzoic acid

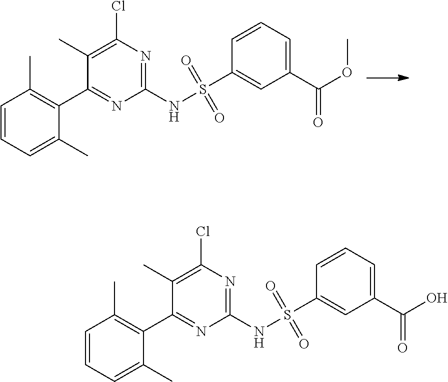 [SMILES: COC(=O)c1cccc(S(=O)(=O)Nc2nc(Cl)c(C)c(-c3c(C)cccc3C)n2)c1 ; Cc1cccc(C)c1-c1nc(NS(=O)(=O)c2cccc(C(=O)O)c2)nc(Cl)c1C]

To a solution of methyl 3-[[4-chloro-6-(2,6-dimethylphenyl)-5-methyl-pyrimidin-2-yl]sulfamoyl]benzoate (59.51 g, 133.45 mmol) in THE (500 mL) was added an aqueous solution of NaOH (300 mL of 2 M, 600.00 mmol) and the mixture was stirred for 2 hours at room temperature. The solution was acidified using 3 M HCl (500 mL) and extracted with ethyl acetate (2×500 mL) before being washed with brine (500 mL). The organic layer was dried over sodium sulfate and concentrated in vacuum. The organic residue was then recrystallized with ethanol and filtered to give 3-[[4-chloro-6-(2,6-dimethylphenyl)-5-methyl-pyrimidin-2-yl]sulfamoyl]benzoic acid (34.44 g, 56%) as a white solid. ¹H NMR (500 MHz, DMSO-d6) δ 8.43 (t, J=1.8 Hz, 1H), 8.18 (dt, J=7.8, 1.4 Hz, 1H), 8.10 (ddd, J=7.9, 2.0, 1.2 Hz, 1H), 7.66 (t, J=7.8 Hz, 1H), 7.25 (t, J=7.6 Hz, 1H), 7.12 (d, J=7.6 Hz, 2H), 1.85 (s, 3H), 1.74 (s, 6H). ESI-MS m/z calc. 431.07065, found 432.4 (M+1)⁺; Retention time: 2.43 minutes; LC method E.

### Example 28: Preparation of 3-[[4-[(2R)-2-amino-4,4-dimethyl-pentoxy]-6-(2,6-dimethylphenyl)-5-methyl-pyrimidin-2-yl]sulfamoyl]benzoic acid

### Step 1: 3-[[4-[(2R)-2-Amino-4,4-dimethyl-pentoxy]-6-(2,6-dimethylphenyl)-5-methyl-pyrimidin-2-yl]sulfamoyl]benzoic acid

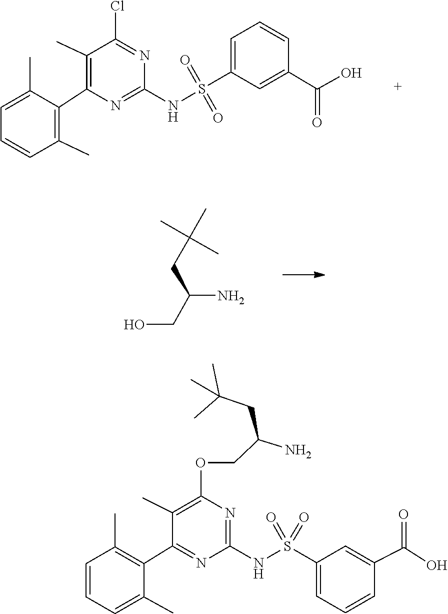

In a 100 mL flask, 3-[[4-chloro-6-(2,6-dimethylphenyl)-5-methyl-pyrimidin-2-yl]sulfamoyl]benzoic acid (872 mg, 2.019 mmol) and (2R)-2-amino-4,4-dimethyl-pentan-1-ol (hydrochloride salt) (341 mg, 2.034 mmol) were charged under nitrogen with anhydrous THE (5 mL) (suspension). Sodium tert-butoxide (770 mg, 8.012 mmol) was added (slight exotherm). The solids slowly dissolved. After 4 hours, another 118 mg of amino alcohol was added, and the mixture was stirred for 1.5 hours. The mixture was partitioned between ethyl acetate (30 mL) and aqueous 1 M HCl (30 mL). After separation, the aqueous phase was further extracted with EtOAc (30 mL). Brine was added to the aqueous phase that still contained large amounts of product and it was extracted with ethyl acetate (30 mL). The combined extracts were dried over sodium sulfate and the solvents evaporated to give crude 3-[[4-[(2R)-2-amino-4,4-dimethyl-pentoxy]-6-(2,6-dimethylphenyl)-5-methyl-pyrimidin-2-yl]sulfamoyl]benzoic acid (hydrochloride salt) (1.12 g, 99%). ESI-MS m/z calc. 526.225, found 527.33 (M+1)⁺; Retention time: 0.45 minutes; LC method B.

### Example 29: Preparation of 3-[[4-[(2R)-2-amino-4,4-dimethyl-pentoxy]-6-[2-(cyclohexylmethyl)-6-methyl-phenyl]-5-methyl-pyrimidin-2-yl]sulfamoyl]benzoic acid

### Step 1: Cyclohexyl(triphenyl)phosphonium bromide

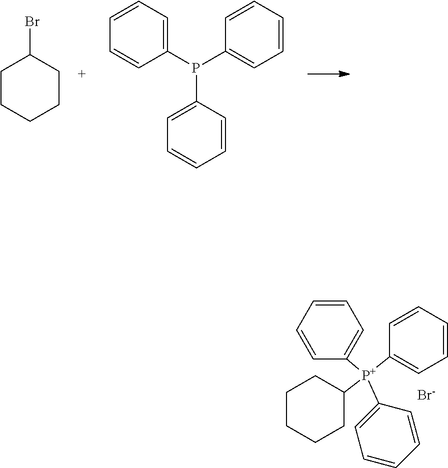 [SMILES: BrC1CCCCC1 ; c1ccc(P(c2ccccc2)c2ccccc2)cc1 ; c1ccc([P+](c2ccccc2)(c2ccccc2)C2CCCCC2)cc1 ; [Br-]]

A sealed Schlenk flask with Teflon tap was charged with triphenylphosphane (70 g, 266.89 mmol) and bromocyclohexane (52.960 g, 40 mL, 324.80 mmol) and the mixture was heated to 165° C. for 24 h. To the resulting yellow oil, toluene was added (100 mL) and the white precipitate was washed with cold THE (300 mL) and cold Et₂O (300 mL). Removal of all the volatiles and drying in vacuo yielded cyclohexyl(triphenyl)phosphonium bromide (77.2 g, 67%) as a white powder. ¹H NMR (500 MHz, Chloroform-d) δ 8.00 (dd, J=11.9, 7.7 Hz, 5H), 7.78-7.64 (m, 8H), 7.59-7.41 (m, 2H), 5.38 (t, J=12.4 Hz, 1H), 2.15 (d, J=12.3 Hz, 4H), 1.85-1.67 (m, 3H), 1.10-0.94 (m, 3H).

### Step 2: (2-Bromo-3-methyl-phenyl)methanol

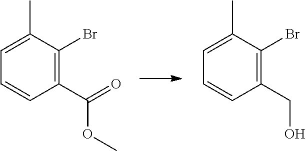

To a solution of LiBH₄ in THE (95.0 mL of 2 M, 190.0 mmol) stirring under nitrogen atmosphere and cooled in an ice-water bath was slowly added a solution of methyl 2-bromo-3-methyl-benzoate (30 g, 130.96 mmol) in ether (200 mL). Upon completion of reagent addition, the mixture was warmed to room temperature and stirred for 16 h. The mixture was quenched by the slow addition of 0.5 N HCl (50 mL) until pH=6-7. The resulting mixture was extracted with diethyl ether and the organic layer was washed with brine, dried (sodium sulfate), filtered and concentrated to give (2-bromo-3-methyl-phenyl)methanol (25.68 g, 93%); ESI-MS m/z calc. 199.9837, found 183.5 (M+1-18)⁺; Retention time: 2.53 minutes; LC Method T.

### Step 3: 2-Bromo-3-methyl-benzaldehyde

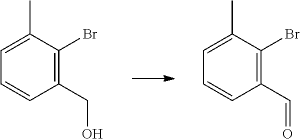

PhI(OAc)₂ (48 g, 149.02 mmol) was added to a solution of (2-bromo-3-methyl-phenyl)methanol (25.68 g, 121.34 mmol) and TEMPO (2.1 g, 13.440 mmol) in DCM (210 mL). The reaction mixture was stirred until the alcohol was no longer detectable (TLC), and then it was diluted with DCM. The mixture was washed with sat. Na₂S₂O₃ solution and extracted with DCM. The combined organic extracts were washed with aq. NaHCO₃, dried over anhydrous Na₂SO₄, and concentrated. The crude was purified by silica gel chromatography (80 g silica, 0-30% gradient of EtOAc in hexanes) to afford 2-bromo-3-methyl-benzaldehyde (22.1 g, 91%) as a white solid.

### Step 4: 2-Bromo-1-(cyclohexylidenemethyl)-3-methyl-benzene

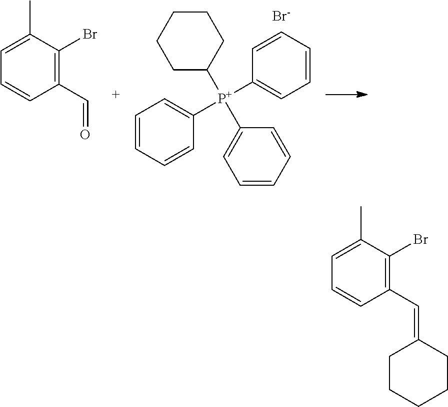

To a suspension of cyclohexyl(triphenyl)phosphonium; bromide (77.2 g, 181.50 mmol) in THE (220 mL) at 0° C. was added potassium tert-butoxide (26.165 g, 233.17 mmol) in THE (220 mL). The mixture was then stirred at RT for 1 h. The mixture was then cooled back down to 0° C. and a solution of 2-bromo-3-methyl-benzaldehyde (22.1 g, 111.03 mmol) in THE (45 mL) was added. The reaction was then stirred at RT for 17 h. The reaction mixture was then quenched with cold water (200 mL) and the aqueous layer was extracted with EtOAc (2×300 mL). The organic layers were then combined, washed with brine, dried with anhydrous Na₂SO₄, filtered, and concentrated under reduced pressure. The crude product was then purified via silica gel column chromatography (eluting with 0 to 5% EtOAc in hexanes) to yield 2-bromo-1-(cyclohexylidenemethyl)-3-methyl-benzene (24.66 g, 82%) as a clear oil. ¹H NMR (500 MHz, Chloroform-d) δ 7.17-7.06 (m, 2H), 7.03 (m, 1H), 6.19 (s, 1H), 2.43 (s, 3H), 2.33-2.25 (m, 2H), 2.24-2.16 (m, 2H), 1.72-1.64 (m, 2H), 1.64-1.58 (m, 2H), 1.55 (m, 2H).

### Step 5: 2-[2-(Cyclohexylidenemethyl)-6-methyl-phenyl]-4,4,5,5-tetramethyl-1,3,2-dioxaborolane

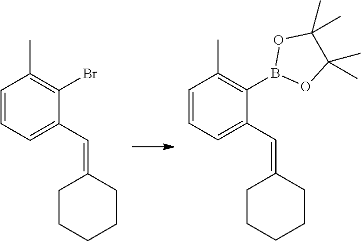 [SMILES: Cc1cccc(C=C2CCCCC2)c1Br ; Cc1cccc(C=C2CCCCC2)c1B1OC(C)(C)C(C)(C)O1]

In a 20-mL sealed tube was added 2-bromo-1-(cyclohexylidenemethyl)-3-methyl-benzene (34.9 g, 125.02 mmol) in dioxane (300 mL), and KOAc (29 g, 295.49 mmol) was added. The resulting mixture was degassed with N₂ for several minutes. Then, 4,4,5,5-tetramethyl-2-(4,4,5,5-tetramethyl-1,3,2-dioxaborolan-2-yl)-1,3,2-dioxaborolane (54 g, 212.65 mmol) was added, followed by Pd(dppf)Cl₂ (9 g, 12.3 mmol), and the reaction was purged again by N₂, sealed and heated at 100° C. for 16 h. Thereafter, the reaction was cooled to room temperature, saturated ammonium chloride solution was added, and the mixture was extracted with ethyl acetate. The combined organic extracts washed with brine, dried over sodium sulfate, filtered and concentrated. The resulting brown oil was purified utilizing silica gel chromatography (330 g×2 of silica, 0-15% gradient of EtOAc in hexanes) to afford 2-[2-(cyclohexylidenemethyl)-6-methyl-phenyl]-4,4,5,5-tetramethyl-1,3,2-dioxaborolane (24 g, 61%) as a yellow oil. ESI-MS m/z calc. 312.22607, found 313.4 (M+1)⁺; Retention time: 4.62 minutes; LC Method E.

### Step 6: tert-Butyl N-tert-butoxycarbonyl-N-[4-chloro-6-[2-(cyclohexylidenemethyl)-6-methyl-phenyl]-5-methyl-pyrimidin-2-yl]carbamate

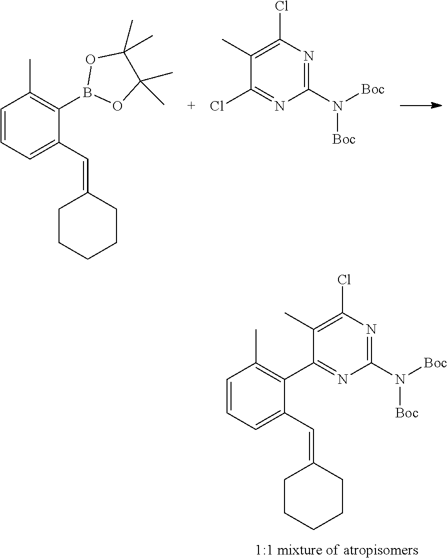 [SMILES: Cc1cccc(C=C2CCCCC2)c1B1OC(C)(C)C(C)(C)O1 ; Cc1c(Cl)nc(N(C(=O)OC(C)(C)C)C(=O)OC(C)(C)C)nc1Cl ; Cc1cccc(C=C2CCCCC2)c1-c1nc(N(C(=O)OC(C)(C)C)C(=O)OC(C)(C)C)nc(Cl)c1C]

To a slurry of tert-butyl N-tert-butoxycarbonyl-N-(4,6-dichloro-5-methyl-pyrimidin-2-yl)carbamate (12.4 g, 31.143 mmol), 2-[2-(cyclohexylidenemethyl)-6-methyl-phenyl]-4,4,5,5-tetramethyl-1,3,2-dioxaborolane (12 g, 36.509 mmol) and Cs₂CO₃ (25 g, 76.730 mmol) in dimethoxyethane (100 mL) and H₂O (20 mL) was added Pd(dppf)Cl₂ (1.1 g, 1.5033 mmol), and the mixture was vigorously stirred under nitrogen at 80° C. (reflux) for 3 h. The reaction mixture was cooled to ambient temperature and diluted with water (100 mL). The aqueous phase was separated and extracted with EtOAc (100 mL). The organic phase was washed with brine (150 mL), dried over anhydrous Na₂SO₄ and concentrated in vacuo. The crude product was purified by silica gel chromatography (330 g of silica, 0-20% gradient of EtOAc in hexane) to afford tert-butyl N-tert-butoxycarbonyl-N-[4-chloro-6-[2-(cyclohexylidenemethyl)-6-methyl-phenyl]-5-methyl-pyrimidin-2-yl]carbamate (8.64 g, 50%) as a colorless oil. ESI-MS m/z calc. 527.25507, found 528.6 (M+1)⁺; Retention time: 4.92 minutes; LC Method E.

### Step 7: 4-Chloro-6-[2-(cyclohexylidenemethyl)-6-methyl-phenyl]-5-methyl-pyrimidin-2-amine

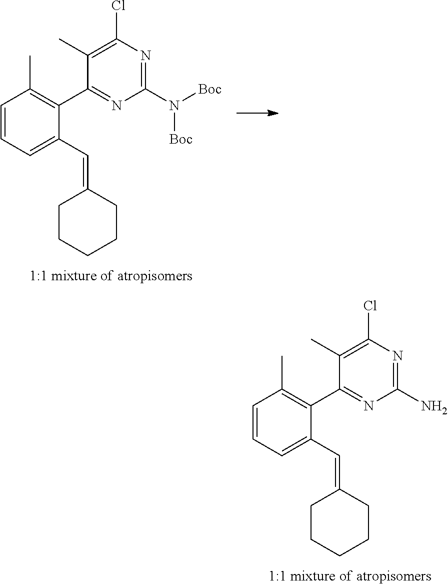 [SMILES: Cc1cccc(C=C2CCCCC2)c1-c1nc(N(C(=O)OC(C)(C)C)C(=O)OC(C)(C)C)nc(Cl)c1C ; Cc1cccc(C=C2CCCCC2)c1-c1nc(N)nc(Cl)c1C]

To a solution of tert-butyl N-tert-butoxycarbonyl-N-[4-chloro-6-[2-(cyclohexylidenemethyl)-6-methyl-phenyl]-5-methyl-pyrimidin-2-yl]carbamate (8.2 g, 14.751 mmol) in DCM (3 mL) was added a solution of HCl in dioxane (5.0 mL of 4 M, 20.0 mmol). The reaction mixture was stirred at ambient temperature for 5 h. Then, sat. NaHCO₃ solution was added. The organic phase was separated, dried and concentrated to afford 4-chloro-6-[2-(cyclohexylidenemethyl)-6-methyl-phenyl]-5-methyl-pyrimidin-2-amine (3.37 g, 69%); ESI-MS m/z calc. 327.15024, found 328.6 (M+1)⁺; Retention time: 3.72 minutes; LC Method E.

### Step 8: Methyl 3-[[4-chloro-6-[2-(cyclohexylidenemethyl)-6-methyl-phenyl]-5-methyl-pyrimidin-2-yl]sulfamoyl]benzoate

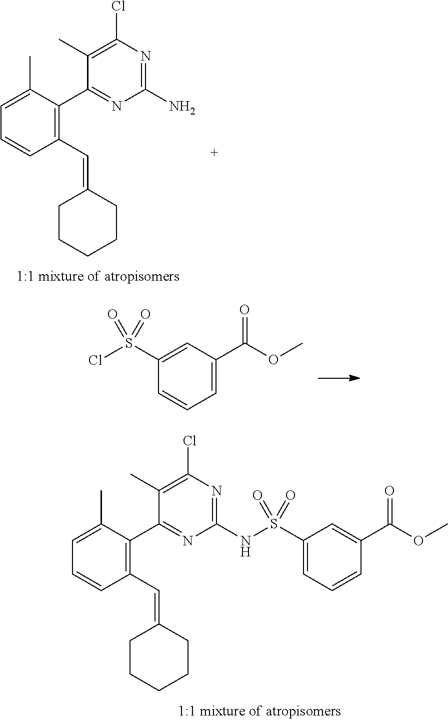

4-Chloro-6-[2-(cyclohexylidenemethyl)-6-methyl-phenyl]-5-methyl-pyrimidin-2-amine (2.6 g, 7.8511 mmol) was dissolved in THF (20 mL) and cooled in an ice bath under nitrogen. To the cold solution, methyl 3-chlorosulfonylbenzoate (2.8 g, 11.932 mmol) in solution in THE (10 mL) was added. At 0° C., lithium tert-pentoxide in heptane (5.5 g, 40% w/w, 23.384 mmol) was added dropwise and the reaction was stirred at room temperature for 2 h. The reaction was quenched with 1 N HCl solution (2 mL). The reaction was diluted with water (3 mL) and EtOAc (5 mL). The organic phase was separated, and the aqueous phase was extracted with EtOAc (5 mL). The combined organic phases were dried over sodium sulfate, filtered, and concentrated. The crude material was purified by silica gel chromatography (330 g silica, 0-40% gradient of acetone in hexane) to give methyl 3-[[4-chloro-6-[2-(cyclohexylidenemethyl)-6-methyl-phenyl]-5-methyl-pyrimidin-2-yl]sulfamoyl]benzoate (2.41 g, 57%) as a white solid. ESI-MS m/z calc. 525.1489, found 526.5 (M+1)⁺; Retention time: 4.24 minutes; LC Method E.

### Step 9: 3-[[4-Chloro-6-[2-(cyclohexylidenemethyl)-6-methyl-phenyl]-5-methyl-pyrimidin-2-yl]sulfamoyl]benzoic acid

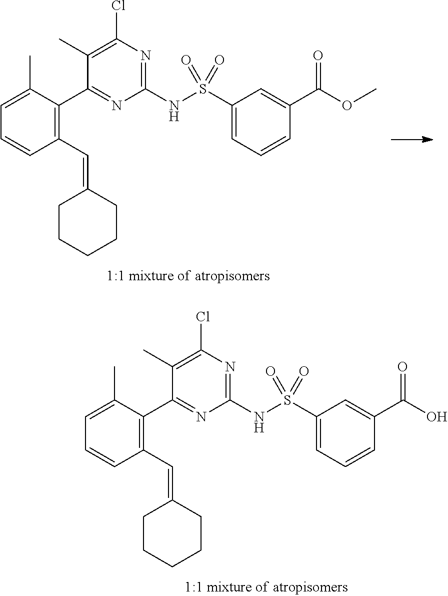 [SMILES: COC(=O)c1cccc(S(=O)(=O)Nc2nc(Cl)c(C)c(-c3c(C)cccc3C=C3CCCCC3)n2)c1 ; Cc1cccc(C=C2CCCCC2)c1-c1nc(NS(=O)(=O)c2cccc(C(=O)O)c2)nc(Cl)c1C]

To a solution of methyl 3-[[4-chloro-6-[2-(cyclohexylidenemethyl)-6-methyl-phenyl]-5-methyl-pyrimidin-2-yl]sulfamoyl]benzoate (1.5 g, 2.7659 mmol) in THE (10 mL) was added an aqueous solution of NaOH (2 mL of 3 M, 6.0 mmol) and stirred for 1 h at room temperature. The solution was acidified using 1 N HCl solution and extracted with ethyl acetate. The combined organic extracts were washed with brine. The organic layer was dried over sodium sulfate and concentrated in vacuo to give 3-[[4-chloro-6-[2-(cyclohexylidenemethyl)-6-methyl-phenyl]-5-methyl-pyrimidin-2-yl]sulfamoyl]benzoic acid (1.2 g, 82%) as a white solid. ESI-MS m/z calc. 511.13324, found 512.3 (M+1)⁺; Retention time: 3.8 minutes; LC Method E.

### Step 10: 3-[[4-[(2R)-2-(tert-Butoxycarbonylamino)-4,4-dimethyl-pentoxy]-6-[2-(cyclohexylidenemethyl)-6-methyl-phenyl]-5-methyl-pyrimidin-2-yl]sulfamoyl]benzoic acid

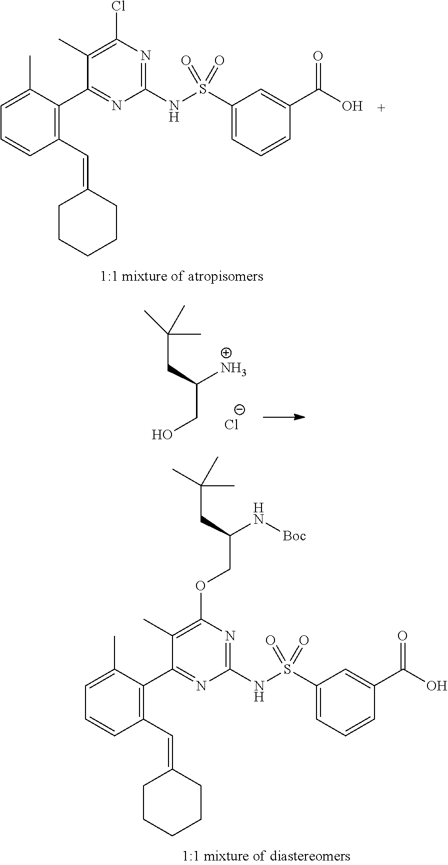

To a stirring solution of [(1R)-1-(hydroxymethyl)-3,3-dimethyl-butyl]ammonium chloride (1 g, 5.9638 mmol) in anhydrous THE (20 mL) at room temperature under nitrogen was added NaOtBu (1.5 g, 15.608 mmol). The reaction mixture was stirred for 10 min, and 3-[[4-chloro-6-[2-(cyclohexylidenemethyl)-6-methyl-phenyl]-5-methyl-pyrimidin-2-yl]sulfamoyl]benzoic acid (1.6 g, 3.0311 mmol) was added. The reaction mixture was stirred at room temperature for 2 h. Then, tert-butoxycarbonyl tert-butyl carbonate (4 g, 18.328 mmol) was added and the reaction mixture was stirred for 3 h. The reaction was quenched with saturated aqueous ammonium chloride (2 mL). The volatiles were removed in vacuo and the aqueous layer was acidified to pH ˜3 with 10% aqueous citric acid. The product was extracted with ethyl acetate, the combined organic layers were dried over anhydrous sodium sulfate and concentrated in vacuo. The residue was purified by silica gel chromatography (120 g silica, 0-40% gradient of acetone in hexane) to yield 3-[[4-[(2R)-2-(tert-butoxycarbonylamino)-4,4-dimethyl-pentoxy]-6-[2-(cyclohexylidenemethyl)-6-methyl-phenyl]-5-methyl-pyrimidin-2-yl]sulfamoyl]benzoic acid (2 g, 75%) as a colorless oil. ESI-MS m/z calc. 706.34, found 707.5 (M+1)⁺; Retention time: 4.13 minutes; LC Method E.

### Step 11: 3-[[4-[(2R)-2-(tert-Butoxycarbonylamino)-4,4-dimethyl-pentoxy]-6-[2-(cyclohexylmethyl)-6-methyl-phenyl]-5-methyl-pyrimidin-2-yl]sulfamoyl]benzoic acid

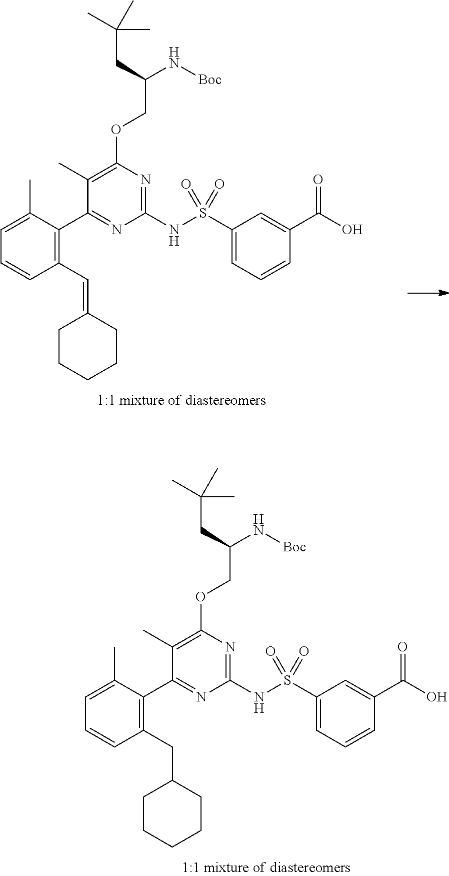 [SMILES: Cc1cccc(C=C2CCCCC2)c1-c1nc(NS(=O)(=O)c2cccc(C(=O)O)c2)nc(OC[C@@H](CC(C)(C)C)NC(=O)OC(C)(C)C)c1C ; Cc1cccc(CC2CCCCC2)c1-c1nc(NS(=O)(=O)c2cccc(C(=O)O)c2)nc(OC[C@@H](CC(C)(C)C)NC(=O)OC(C)(C)C)c1C]

3-[[4-[(2R)-2-(tert-Butoxycarbonylamino)-4,4-dimethyl-pentoxy]-6-[2-(cyclohexylidenemethyl)-6-methyl-phenyl]-5-methyl-pyrimidin-2-yl]sulfamoyl]benzoic acid (2 g, 2.2634 mmol) in MeOH (30 mL) was charged with Pd/C (25 mg). The reaction was stirred at rt for 12 h under a balloon of H₂. After completion, the reaction was filtered through Celite and concentrated in vacuo. The crude material was purified by reverse-phase HPLC (gradient 30-85% acetonitrile in water buffered by 5 mM HCl) to afford 3-[[4-[(2R)-2-(tert-butoxycarbonylamino)-4,4-dimethyl-pentoxy]-6-[2-(cyclohexylmethyl)-6-methyl-phenyl]-5-methyl-pyrimidin-2-yl]sulfamoyl]benzoic acid (1.25 g, 77%) as a white solid. ESI-MS m/z calc. 708.35565, found 709.6 (M+1)⁺; Retention time: 4.23 minutes; LC Method E.

### Step 12: 3-[[4-[(2R)-2-Amino-4,4-dimethyl-pentoxy]-6-[2-(cyclohexylmethyl)-6-methyl-phenyl]-5-methyl-pyrimidin-2-yl]sulfamoyl]benzoic acid

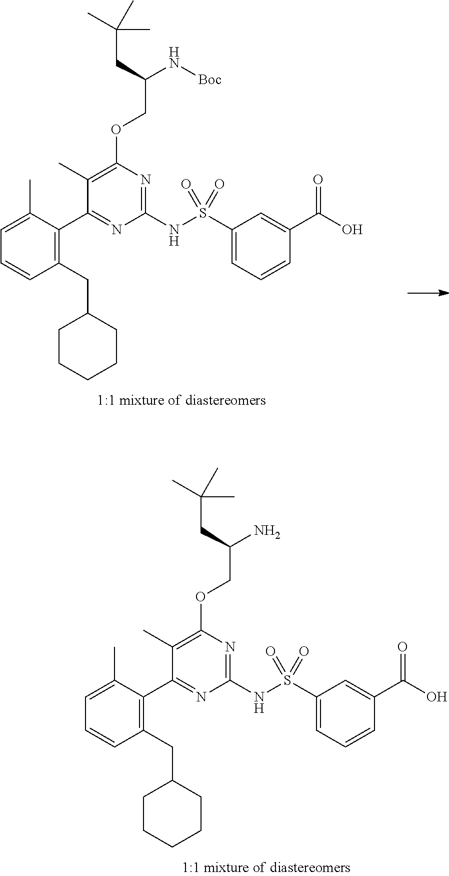 [SMILES: Cc1cccc(CC2CCCCC2)c1-c1nc(NS(=O)(=O)c2cccc(C(=O)O)c2)nc(OC[C@@H](CC(C)(C)C)NC(=O)OC(C)(C)C)c1C ; Cc1cccc(CC2CCCCC2)c1-c1nc(NS(=O)(=O)c2cccc(C(=O)O)c2)nc(OC[C@H](N)CC(C)(C)C)c1C]

To a solution of 3-[[4-[(2R)-2-(tert-butoxycarbonylamino)-4,4-dimethyl-pentoxy]-6-[2-(cyclohexylmethyl)-6-methyl-phenyl]-5-methyl-pyrimidin-2-yl]sulfamoyl]benzoic acid (1.25 g, 1.7456 mmol) in DCM (10 mL) was added a solution of HCl in dioxane (2 mL of 4 M, 8.0 mmol). The reaction mixture was stirred at room temperature for 5 h. After completion, the volatile material was evaporated in vacuo to afford 3-[[4-[(2R)-2-amino-4,4-dimethyl-pentoxy]-6-[2-(cyclohexylmethyl)-6-methyl-phenyl]-5-methyl-pyrimidin-2-yl]sulfamoyl]benzoic acid (hydrochloride salt) (1.1 g, 94%) as a white solid. ¹H NMR (500 MHz, DMSO-d6) δ 8.42 (q, J=1.5, 1.5, 1.1 Hz, 1H), 8.29 (d, J=5.7 Hz, 3H), 8.15-8.04 (m, 2H), 7.66 (td, J=7.8, 2.7 Hz, 1H), 7.33-7.22 (m, 1H), 7.17-7.04 (m, 2H), 4.38-4.19 (m, 1H), 4.12 (apparent ddd, J=24.6, 12.0, 5.8 Hz, 2H), 2.19-2.02 (m, 1H), 2.07-1.94 (m, 1H), 1.90 (apparent d, J=22.8 Hz, 3H), 1.67 (d, J=4.0 Hz, 3H), 1.66-1.44 (m, 5H), 1.44-1.14 (m, 3H), 1.10-0.93 (m, 3H), 0.90 (d, J=16.2 Hz, 9H), 0.68 (q, J=12.0, 2H). ESI-MS m/z calc. 608.3032, found 609.5 (M+1)⁺; Retention time: 2.29 minutes; LC Method E.

### Example 30: Preparation of 3-[[4-[(2R)-2-amino-4,4-dimethyl-pentoxy]-6-(2-isopropyl-6-methyl-phenyl)-5-methyl-pyrimidin-2-yl]sulfamoyl]benzoic acid

### Step 1: 2-Bromo-1-isopropyl-3-methyl-benzene

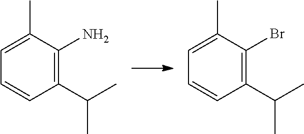

Into a solution of 2-isopropyl-6-methyl-aniline (10.048 g, 10.5 mL, 67.331 mmol) in concentrated HBr (100 mL) and water (100 mL) was added a solution of sodium nitrite (5.6 g, 81.165 mmol) in water (40 mL) at 0° C. After the addition, the reaction was stirred at 0° C. for 20 min. In a separate flask, a mixture of CuBr (9.67 g, 67.410 mmol) in concentrated HBr (100 mL) was heated to 60° C. The diazonium salt solution was added dropwise onto the CuBr mixture. After the addition was complete, the reaction was stirred at 60° C. for 1 h, and then it was cooled to rt. The solution was extracted with diethyl ether (3×100 mL). The combined ether layers were washed with sat. sodium bicarbonate (100 mL) and brine (75 mL), dried over anhydrous sodium sulfate and concentrated in vacuo. The residue was directly loaded onto a silica gel column and purified using 0 to 40% diethyl ether in hexane to furnish 2-bromo-1-isopropyl-3-methyl-benzene (7 g, 46%) as a clear oil. ¹H NMR (500 MHz, Chloroform-d) δ 7.18 (m, 1H), 7.15-7.11 (m, 1H), 7.11-7.06 (m, 1H), 3.59-3.42 (m, 1H), 2.44 (d, J=1.9 Hz, 3H), 1.26 (d, J=1.8 Hz, 3H), 1.25 (d, J=2.0 Hz, 3H).

### Step 2: (2-Isopropyl-6-methyl-phenyl)boronic acid

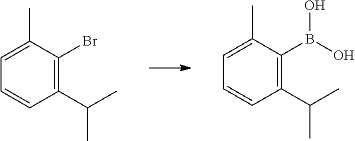

To a solution of 2-bromo-1-isopropyl-3-methyl-benzene (5.4 g, 25.339 mmol) in anhydrous THE (150 mL) at −78° C. was added dropwise a solution of n-BuLi in hexanes (12 mL of 2.5 M, 30.0 mmol). The solution was stirred at −78° C. for 15 min before adding trimethyl borate (7.9220 g, 8.5 mL, 76.237 mmol) dropwise. After the addition, the solution was allowed to warm to 0° C. and was stirred for 1 h. The solution was then quenched with 1 N HCl solution, and this was stirred for 3 h and then partitioned with EtOAc. The aqueous layer was extracted with EtOAc (2×20 mL). The organic layers were then combined, washed with brine, dried with anhydrous sodium sulfate, filtered, and concentrated under reduced pressure. The crude material was purified via silica gel column chromatography (eluting with 0-40% EtOAc in hexanes) to yield (2-isopropyl-6-methyl-phenyl)boronic acid (2.156 g, 45%) as a white solid. ¹H NMR (250 MHz, DMSO-d6) δ 8.21-8.07 (m, 1H), 7.14 (t, J=7.5, 7.5 Hz, 1H), 7.03 (d, J=7.7 Hz, 1H), 6.92 (d, J=7.3 Hz, 1H), 2.82 (m, 1H), 2.26 (s, 3H), 1.39-0.96 (m, 6H).

### Step 3: tert-Butyl N-tert-butoxycarbonyl-N-[4-chloro-6-(2-isopropyl-6-methyl-phenyl)-5-methyl-pyrimidin-2-yl]carbamate

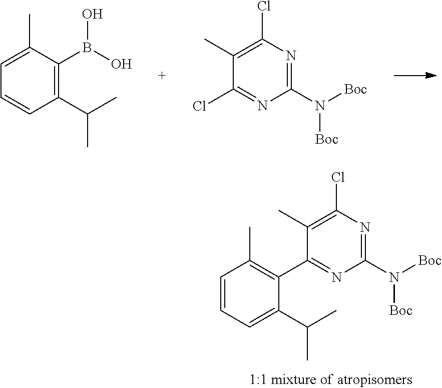

To a mixture of tert-butyl N-tert-butoxycarbonyl-N-(4,6-dichloro-5-methyl-pyrimidin-2-yl)carbamate (7.78 g, 20.568 mmol), (2-isopropyl-6-methyl-phenyl)boronic acid (3.2 g, 17.974 mmol), cesium carbonate (14.5 g, 44.503 mmol), and Pd(dppf)Cl₂ (1.47 g, 1.8001 mmol) was added a degassed solvent mixture of dimethoxyethane (70 mL) and water (70 mL). During the addition of the solvents, the reaction vial was continuously flushed with nitrogen. Once the addition was finished, the vial was sealed and heated to 80° C., and the reaction mixture was stirred for 2 h. The mixture was then quenched with water (40 mL) and EtOAc (70 mL). The aqueous layer was extracted with EtOAc (2×50 mL). The organic layers were combined, washed with brine, dried with anhydrous sodium sulfate, filtered, and concentrated under reduced pressure. The crude was purified via silica gel column chromatography (eluting with a gradient of 0 to 30% EtOAc in hexanes) to yield tert-butyl N-tert-butoxycarbonyl-N-[4-chloro-6-(2-isopropyl-6-methyl-phenyl)-5-methyl-pyrimidin-2-yl]carbamate (5.36 g, 41%) as a white solid. ESI-MS m/z calc. 475.2238, found 476.2 (M+1)⁺; Retention time: 4.14 minutes; LC Method E.

### Step 4: 4-Chloro-6-(2-isopropyl-6-methyl-phenyl)-5-methyl-pyrimidin-2-amine

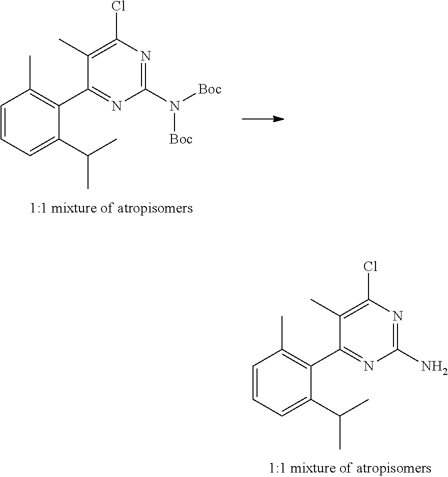 [SMILES: Cc1cccc(C(C)C)c1-c1nc(N(C(=O)OC(C)(C)C)C(=O)OC(C)(C)C)nc(Cl)c1C ; Cc1cccc(C(C)C)c1-c1nc(N)nc(Cl)c1C]

To a solution of tert-butyl N-tert-butoxycarbonyl-N-[4-chloro-6-(2-isopropyl-6-methyl-phenyl)-5-methyl-pyrimidin-2-yl]carbamate (5.36 g, 7.3192 mmol) in anhydrous DCM (30 mL) at 0° C. was added a solution of HCl in dioxane (30 mL of 4 M, 120.0 mmol). The solution was then warmed to RT and stirred for 3 h. The solution was then quenched with NaHCO₃ (75 mL) and partitioned with DCM (50 mL). The aqueous layer was extracted with DCM (2×50 mL). The organic layers were then combined, washed with brine, dried with anhydrous sodium sulfate, filtered, and concentrated under reduced pressure. The crude product was purified via silica gel column chromatography (eluting with a gradient of 0 to 40% ether in hexanes) to yield 4-chloro-6-(2-isopropyl-6-methyl-phenyl)-5-methyl-pyrimidin-2-amine (2.5 g, >100% yield) as a white solid. ¹H NMR (500 MHz, DMSO-d6) δ 7.33-7.18 (m, 2H), 7.10 (d, J=7.3 Hz, 1H), 6.91 (s, 2H), 1.93 (s, 3H), 1.77 (s, 3H), 1.07 (apparent dd, J=16.5, 6.8 Hz, 6H). ESI-MS m/z calc. 275.11893, found 276.3 (M+1)⁺; Retention time: 2.5 minutes; LC Method H.

### Step 5: Methyl 3-[[4-chloro-6-(2-isopropyl-6-methyl-phenyl)-5-methyl-pyrimidin-2-yl]sulfamoyl]benzoate

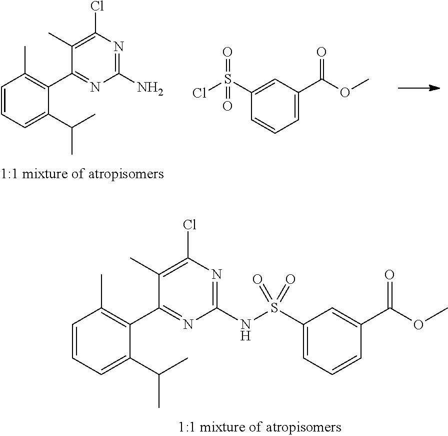 [SMILES: Cc1cccc(C(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3C(C)C)n2)c1]

To a solution of 4-chloro-6-(2-isopropyl-6-methyl-phenyl)-5-methyl-pyrimidin-2-amine (2.5 g, 8.3401 mmol) and methyl 3-chlorosulfonylbenzoate (2.57 g, 10.952 mmol) in anhydrous THE (25 mL) at 0° C. was added dropwise a solution of lithium tert-amoxide in heptanes (5.2560 g, 18 mL of 40% w/w, 22.347 mmol), which was then stirred at 0° C. for 5 min after addition. The solution was then warmed to RT and stirred for 2 h. The reaction was then quenched with 1 N HCl solution (50 mL) and partitioned with EtOAc (50 mL). The aqueous layer was extracted with EtOAc (2×50 mL). The organic layers were then combined, washed with brine, dried with anhydrous sodium sulfate, filtered, and concentrated under reduced pressure to yield methyl 3-[[4-chloro-6-(2-isopropyl-6-methyl-phenyl)-5-methyl-pyrimidin-2-yl]sulfamoyl]benzoate (4.75 g, 65%) as a yellow solid. ESI-MS m/z calc. 473.1176, found 474.1 (M+1)⁺; Retention time: 3.57 minutes; LC Method E.

### Step 6: 3-[[4-Chloro-6-(2-isopropyl-6-methyl-phenyl)-5-methyl-pyrimidin-2-yl]sulfamoyl]benzoic acid

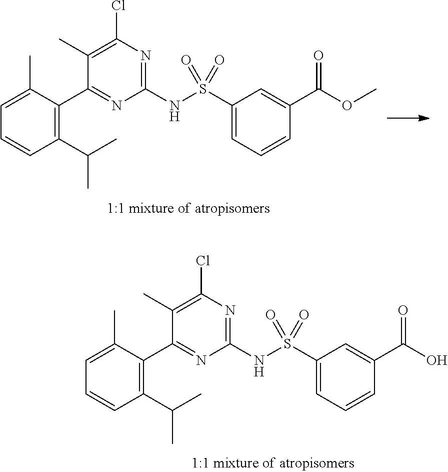 [SMILES: COC(=O)c1cccc(S(=O)(=O)Nc2nc(Cl)c(C)c(-c3c(C)cccc3C(C)C)n2)c1 ; Cc1cccc(C(C)C)c1-c1nc(NS(=O)(=O)c2cccc(C(=O)O)c2)nc(Cl)c1C]

To a solution of methyl 3-[[4-chloro-6-(2-isopropyl-6-methyl-phenyl)-5-methyl-pyrimidin-2-yl]sulfamoyl]benzoate (4.75 g, 5.4117 mmol) in THE (17 mL) was added aqueous NaOH (30 mL of 1 M, 30.0 mmol). The reaction was stirred at RT for 4 h. The reaction was then quenched with 1 N HCl solution (50 mL), and partitioned with EtOAc (30 mL). The aqueous layer was then extracted with EtOAc (2×20 mL). The organic layers were then combined, washed with brine, dried with anhydrous sodium sulfate, filtered, and concentrated under reduced pressure. The crude was purified via reverse-phase HPLC (gradient 60-100% acetonitrile in water buffered by 0.1% TFA) and then by silica gel column chromatography (eluting with a gradient of 0-70% acetone in hexanes buffered by 0.1% AcOH) to yield 3-[[4-chloro-6-(2-isopropyl-6-methyl-phenyl)-5-methyl-pyrimidin-2-yl]sulfamoyl]benzoic acid (2.28 g, 73%) as a white solid. ¹H NMR (500 MHz, DMSO-d6) δ 13.39 (s, 1H), 12.24 (s, 1H), 8.39 (d, J=1.8 Hz, 1H), 8.13 (m, 1H), 8.06 (m, 1H), 7.62 (t, J=7.8, 7.8 Hz, 1H), 7.31 (t, J=7.7, 7.7 Hz, 1H), 7.22 (d, J=7.8 Hz, 1H), 7.09 (d, J=7.4 Hz, 1H), 2.17 (m, 1H), 1.83 (s, 3H), 1.64 (s, 3H), 1.04 (d, J=6.8 Hz, 3H), 0.86 (d, J=6.8 Hz, 3H). ESI-MS m/z calc. 459.10196, found 460.3 (M+1)⁺; Retention time: 2.69 minutes; LC Method H.

### Step 7: 3-[[4-[(2R)-2-amino-4,4-dimethyl-pentoxy]-6-(2-isopropyl-6-methyl-phenyl)-5-methyl-pyrimidin-2-yl]sulfamoyl]benzoic acid

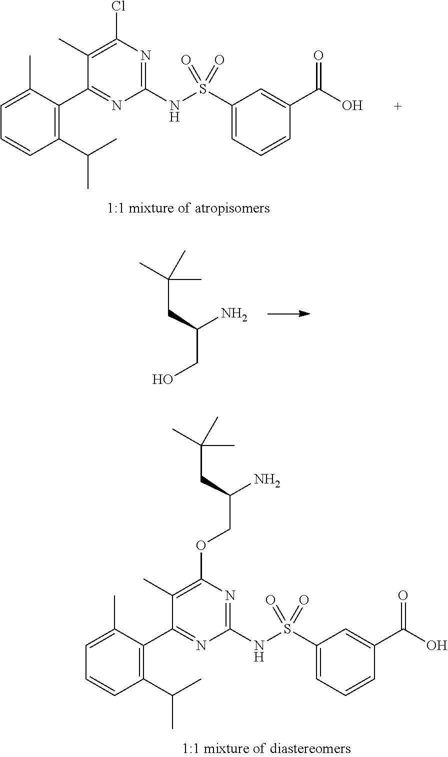

To a solution of 3-[[4-chloro-6-(2-isopropyl-6-methyl-phenyl)-5-methyl-pyrimidin-2-yl]sulfamoyl]benzoic acid (1.07 g, 2.2380 mmol) and (2R)-2-amino-4,4-dimethyl-pentan-1-ol (282 mg, 2.1491 mmol) in anhydrous THE (15 mL) was added sodium tert-butoxide (895 mg, 9.3129 mmol). The reaction was stirred at RT for 2 h. The reaction was then quenched with 1 N HCl solution (35 mL) and partitioned with EtOAc (35 mL). The aqueous layer was then extracted with EtOAc (2×30 mL). The organic layers were combined, washed with brine, dried with anhydrous sodium sulfate, filtered, and concentrated under reduced pressure. The crude product was then purified via reverse-phase column chromatography (gradient 30-70% acetonitrile in water buffered by 0.1% TFA). The pure fractions were concentrated until some water was remaining, and the solution was mixed with 1 N HCl solution (80 mL) to displace the TFA to make the HCl salt. The solution was dried via lyophilizer to yield 3-[[4-[(2R)-2-amino-4,4-dimethyl-pentoxy]-6-(2-isopropyl-6-methyl-phenyl)-5-methyl-pyrimidin-2-yl]sulfamoyl]benzoic acid (hydrochloride salt) (827.5 mg, 60%) as a white solid. ¹H NMR (500 MHz, DMSO-d6) δ 8.43 (m, 1H), 8.21-8.05 (m, 4H), 7.66 (t, J=7.8, 7.8 Hz, 1H), 7.34 (t, J=7.7, 7.7 Hz, 1H), 7.26 (d, J=7.9 Hz, 1H), 7.14 (d, J=7.5 Hz, 1H), 4.36-4.27 (m, 1H), 4.24 (d, J=11.9 Hz, 1H), 3.57 (s, 1H), 2.40-2.31 (m, 1H), 1.88 (s, 3H), 1.67 (s, 3H), 1.62 (m, 1H), 1.52 (m, 1H), 1.05 (m, 6H), 0.93 (d, J=1.3 Hz, 9H). ESI-MS m/z calc. 554.2563, found 555.4 (M+1)⁺; Retention time: 2.03 minutes; LC Method W.

### Example 31: Preparation of 3-[[4-[(2R)-2-amino-4,4-dimethyl-pentoxy]-6-(2-isobutyl-6-methyl-phenyl)-5-methyl-pyrimidin-2-yl]sulfamoyl]benzoic acid

### Step 1: 1-Methyl-3-(2-methylprop-1-enyl)-2-nitro-benzene

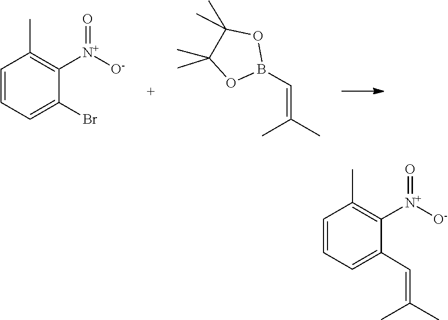

To a stirring solution of 1-bromo-3-methyl-2-nitro-benzene (27.78 g, 128.59 mmol) and 4,4,5,5-tetramethyl-2-(2-methylprop-1-enyl)-1,3,2-dioxaborolane (24.75 g, 135.94 mmol) in 1,4-dioxane (650 mL) at room temperature was added water (200 mL). The reaction mixture was degassed with nitrogen for 30 min. Under nitrogen, sodium carbonate (40.986 g, 386.70 mmol) and [1,1′-bis(diphenylphosphino)ferrocene]dichloropalladium(II) (9.082 g, 12.412 mmol) were added. The reaction mixture was heated to reflux (110° C.) under nitrogen for 18 h. Then, after cooling to room temperature, water (200 mL) and ethyl acetate (400 mL) were added, and the reaction mixture was vigorously stirred for 10 min. Two layers were separated, and the aqueous layer was extracted with ethyl acetate (2×300 mL). The combined organic layers were washed with brine (150 mL), dried over anhydrous sodium sulfate and concentrated. The crude was purified by silica gel chromatography using 0-5% ethyl acetate gradient in hexanes to afford 1-methyl-3-(2-methylprop-1-enyl)-2-nitro-benzene (20.2 g, 76%) as a yellow oil. ¹H NMR (500 MHz, DMSO-d6) δ 7.46 (t, J=7.7 Hz, 1H), 7.34 (d, J=7.7 Hz, 1H), 7.23 (d, J=7.6 Hz, 1H), 6.06 (s, 1H), 2.25 (s, 3H), 1.85 (d, J=1.4 Hz, 3H), 1.68 (d, J=1.4 Hz, 3H). ESI-MS m/z calc. 191.09464, Retention time: 6.12 minutes; LC Method D.

### Step 2: 2-Isobutyl-6-methyl-aniline

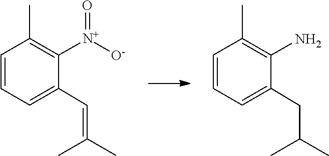

To a stirring solution of 1-methyl-3-(2-methylprop-1-enyl)-2-nitro-benzene (20.2 g, 100.35 mmol) in methanol (300 mL) at room temperature under ambient conditions was added palladium on carbon (10.28 g, 10% w/w, 9.6598 mmol). The head space was purged with nitrogen, followed by hydrogen. The reaction mixture was stirred under the atmosphere of hydrogen (1 atm) for 30 h. The reaction mixture was filtered through a pad of Celite, and the filter cake was washed with methanol (3×50 mL). The combined filtrate was concentrated under vacuum. The crude was purified by silica gel chromatography using 0-5% ethyl acetate gradient in hexanes to afford 2-isobutyl-6-methyl-aniline (16.06 g, 93%) as an amber oil. ¹H NMR (500 MHz, DMSO-d6) δ 6.79 (d, J=7.4 Hz, 1H), 6.72 (d, J=7.4 Hz, 1H), 6.42 (t, J=7.4 Hz, 1H), 4.41 (s, 2H), 2.33 (d, J=7.3 Hz, 2H), 2.06 (s, 3H), 1.91-1.80 (m, 1H), 0.87 (d, J=6.6 Hz, 6H). ESI-MS m/z calc. 163.1361, found 164.4 (M+1)⁺; Retention time: 2.96 minutes; LC Method D.

### Step 3: 2-Bromo-1-isobutyl-3-methyl-benzene

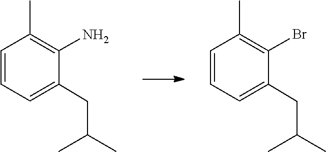

To a stirring suspension of 2-isobutyl-6-methyl-aniline (16.428 g, 100.63 mmol) in a mixture of concentrated (48%) HBr (140 mL) and water (140 mL) at 0° C. was added dropwise a solution of sodium nitrite (8.331 g, 120.75 mmol) in water (60 mL). After the addition was complete, the reaction mixture was stirred at 0° C. for 45 min. To the reaction mixture was slowly added a solution of copper(I) bromide (17.332 g, 120.82 mmol) in concentrated (48%) HBr (140 mL). After the addition was complete, the reaction mixture was heated to 60° C. for 1 h. After cooling to room temperature, diethyl ether (300 mL) was added and the reaction mixture was stirred for 15 min. The layers were separated, and the aqueous layer was extracted with diethyl ether (2×150 mL). The combined organic layers were washed with brine (100 mL), dried over anhydrous sodium sulfate and concentrated. The crude was purified by silica gel chromatography using hexanes to afford 2-bromo-1-isobutyl-3-methyl-benzene (11.77 g, 44%) as a colorless oil. ¹H NMR (500 MHz, DMSO-d) δ 7.22-7.15 (m, 2H), 7.10-7.06 (m, 1H), 2.60 (d, J=7.2 Hz, 2H), 2.36 (s, 3H), 1.98-1.87 (m, 1H), 0.89 (d, J=6.6 Hz, 6H).

### Step 4: (2-Isobutyl-6-methyl-phenyl)boronic acid

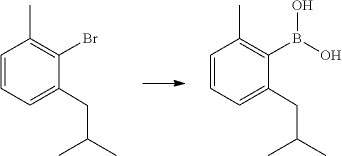

To a stirring solution of 2-bromo-1-isobutyl-3-methyl-benzene (11.77 g, 46.636 mmol) in anhydrous THE (200 mL) at −78° C. under nitrogen was added dropwise a solution of n-butyllithium (23 mL of 2.5 M, 57.500 mmol) in hexanes. After the addition was complete, the reaction mixture was stirred at −78° C. for 1 h. To the reaction mixture was added dropwise trimethyl borate (19.530 g, 21 mL, 187.95 mmol). After the addition was complete, the reaction mixture was stirred at −78° C. for 15 min, then allowed to warm to 0° C. and stirred for 1 h. To the reaction mixture was slowly added aqueous HCl (150 mL of 1 M, 150.0 mmol). After the addition was complete, the reaction mixture was allowed to warm to room temperature and stirred for 3 h. Ethyl acetate (200 mL) was added, and the reaction mixture was vigorously stirred for 10 min. The layers were separated, and the aqueous layer was extracted with ethyl acetate (2×120 mL). The combined organic layers were washed with brine (100 mL), dried over anhydrous sodium sulfate and concentrated. The crude was purified by silica gel chromatography using a 0-20% gradient of acetone in hexanes. The product was repurified by silica gel chromatography using a gradient of 0-40% diethyl ether in hexanes to afford (2-isobutyl-6-methyl-phenyl)boronic acid (3.2 g, 32%) as a white solid. ¹H NMR (500 MHz, DMSO-d6) δ 8.06 (s, 2H), 7.08 (t, J=7.5 Hz, 1H), 6.92 (d, J=7.6 Hz, 1H), 6.87 (d, J=7.6 Hz, 1H), 2.43 (d, J=7.2 Hz, 2H), 2.27 (s, 3H), 1.92-1.81 (m, 1H), 0.85 (d, J=6.6 Hz, 6H). ESI-MS m/z calc. 192.13216, Retention time: 3.98 minutes; LC Method D.

### Step 5: tert-Butyl N-tert-butoxycarbonyl-N-[4-chloro-6-(2-isobutyl-6-methyl-phenyl)-5-methyl-pyrimidin-2-yl]carbamate and tert-butyl N-[4-chloro-6-(2-isobutyl-6-methyl-phenyl)-5-methyl-pyrimidin-2-yl]carbamate

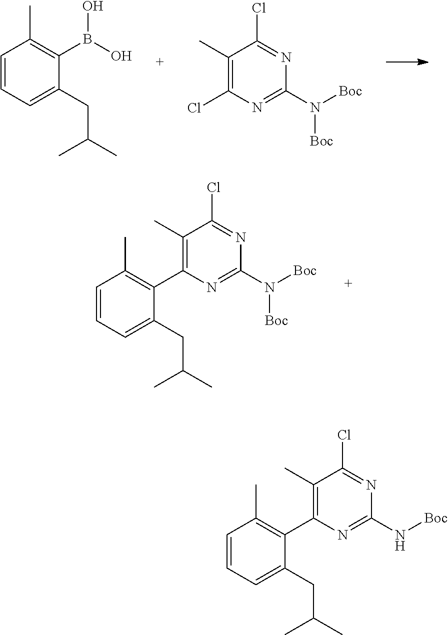 [SMILES: Cc1cccc(CC(C)C)c1B(O)O ; Cc1c(Cl)nc(N(C(=O)OC(C)(C)C)C(=O)OC(C)(C)C)nc1Cl ; Cc1cccc(CC(C)C)c1-c1nc(N(C(=O)OC(C)(C)C)C(=O)OC(C)(C)C)nc(Cl)c1C ; Cc1cccc(CC(C)C)c1-c1nc(NC(=O)OC(C)(C)C)nc(Cl)c1C]

To a stirring solution of tert-butyl N-tert-butoxycarbonyl-N-(4,6-dichloro-5-methyl-pyrimidin-2-yl)carbamate (6.235 g, 16.484 mmol) and (2-isobutyl-6-methyl-phenyl)boronic acid (3.2 g, 14.995 mmol) in 1,2-dimethoxyethane (81 mL) at room temperature was added water (27 mL). The reaction mixture was degassed with nitrogen for 20 min. Under nitrogen, cesium carbonate (14.644 g, 44.945 mmol) and [1,1′-bis(diphenylphosphino)ferrocene]dichloro palladium(II) (1.101 g, 1.5047 mmol) were added. The reaction mixture was heated at 95° C. for 3 h. After cooling to room temperature, water (100 mL) and ethyl acetate (100 mL) were added, and the reaction mixture was vigorously stirred for 5 min. Two layers were separated, and the aqueous layer was extracted with ethyl acetate (2×80 mL). The combined organic layers were washed with brine (60 mL), dried over anhydrous sodium sulfate and concentrated. The crude product was purified by silica gel chromatography using 0-15% diethyl ether gradient in hexanes, then by reverse-phase HPLC using a gradient of 85-100% acetonitrile in water (0.15% THE buffer). The pure product fractions were combined and basified with saturated aqueous NaHCO₃ to pH ˜8. The volatiles were removed in vacuo, and the residual aqueous layer was extracted with ethyl acetate (3×80 mL). The combined organic layers were washed with brine (40 mL), dried over anhydrous sodium sulfate and concentrated to afford a mixture of: tert-butyl N-tert-butoxycarbonyl-N-[4-chloro-6-(2-isobutyl-6-methyl-phenyl)-5-methyl-pyrimidin-2-yl]carbamate (3.1 g, 40%); ESI-MS m/z calc. 489.23944, found 490.3 (M+1)⁺; Retention time: 8.39 minutes; LC Method D, as well as tert-butyl N-[4-chloro-6-(2-isobutyl-6-methyl-phenyl)-5-methyl-pyrimidin-2-yl]carbamate (0.897 g, 15%); ESI-MS m/z calc. 389.187, found 390.6 (M+1)⁺; Retention time: 7.49 minutes; LC Method D.

### Step 6: 4-Chloro-6-(2-isobutyl-6-methyl-phenyl)-5-methyl-pyrimidin-2-amine

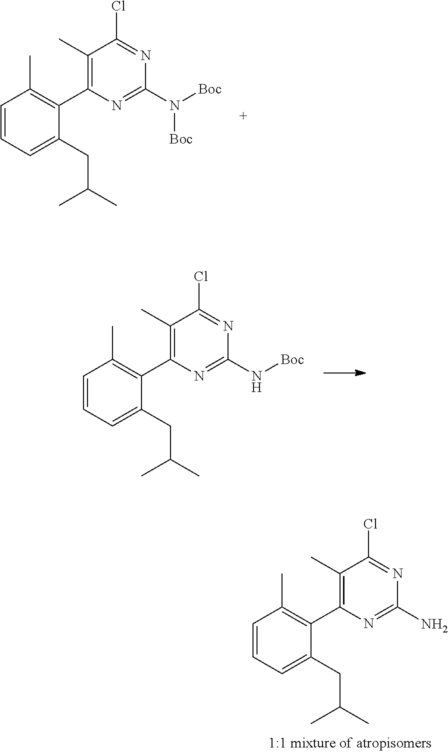 [SMILES: Cc1cccc(CC(C)C)c1-c1nc(N(C(=O)OC(C)(C)C)C(=O)OC(C)(C)C)nc(Cl)c1C ; Cc1cccc(CC(C)C)c1-c1nc(NC(=O)OC(C)(C)C)nc(Cl)c1C ; Cc1cccc(CC(C)C)c1-c1nc(N)nc(Cl)c1C]

To a mixture of tert-butyl N-tert-butoxycarbonyl-N-[4-chloro-6-(2-isobutyl-6-methyl-phenyl)-5-methyl-pyrimidin-2-yl]carbamate and tert-butyl N-[4-chloro-6-(2-isobutyl-6-methyl-phenyl)-5-methyl-pyrimidin-2-yl]carbamate (3.997 g, 8.1566 mmol) in DCM (50 mL) at room temperature was slowly added a solution of HCl (13 mL of 4 M, 52.0 mmol) in dioxane. After the addition was complete, the reaction mixture was sealed and stirred for 18 h. The volatiles were removed in vacuo, then the obtained residue was resuspended in saturated aqueous NaHCO₃ (150 mL) and stirred vigorously for 10 min. Ethyl acetate (120 mL) was added, and the mixture was vigorously stirred for 10 min (until all the solid was dissolved). The layers were separated, and the aqueous layer was extracted with ethyl acetate (2×100 mL). The combined organic layers were washed with brine (60 mL), dried over anhydrous sodium sulfate and concentrated to afford 4-chloro-6-(2-isobutyl-6-methyl-phenyl)-5-methyl-pyrimidin-2-amine (2.349 g, 94%) as a white solid. ESI-MS m/z calc. 289.13458, found 290.3 (M+1)⁺; Retention time: 5.67 minutes; LC Method D.

### Step 7: Methyl 3-[[4-chloro-6-(2-isobutyl-6-methyl-phenyl)-5-methyl-pyrimidin-2-yl]sulfamoyl]benzoate

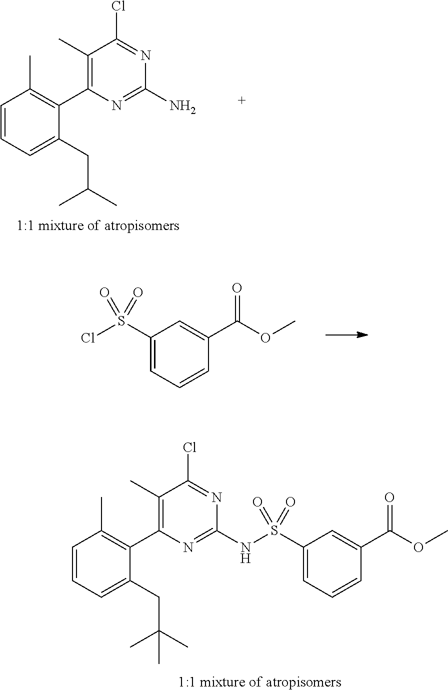 [SMILES: Cc1cccc(CC(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3CC(C)(C)C)n2)c1]

To a stirring solution of 4-chloro-6-(2-isobutyl-6-methyl-phenyl)-5-methyl-pyrimidin-2-amine (2.344 g, 8.0883 mmol) and methyl 3-chlorosulfonylbenzoate (4.812 g, 20.507 mmol) in anhydrous THE (50 mL) at −78° C. under nitrogen was added dropwise a THE solution of LiHMDS (23 mL of 1.3 M, 29.9 mmol). After the addition was complete, the reaction mixture was stirred at −78° C. for 2 h. The reaction mixture was quenched cold with 1 N aqueous HCl (100 mL), and then allowed to warm to room temperature. Ethyl acetate (120 mL) was added, and the reaction mixture was vigorously stirred for 10 min. The layers were separated, and the aqueous layer was extracted with ethyl acetate (2×100 mL). The combined organic layers were washed with brine (60 mL), dried over anhydrous sodium sulfate and concentrated. The crude was purified by silica gel chromatography using a gradient of 0-15% ethyl acetate in hexanes to afford methyl 3-[[4-chloro-6-(2-isobutyl-6-methyl-phenyl)-5-methyl-pyrimidin-2-yl]sulfamoyl]benzoate (3.791 g, 94%) as a white foam. ESI-MS m/z calc. 487.13324, found 488.4 (M+1)⁺; Retention time: 6.96 minutes; LC Method D.

### Step 8: 3-[[4-Chloro-6-(2-isobutyl-6-methyl-phenyl)-5-methyl-pyrimidin-2-yl]sulfamoyl]benzoic acid

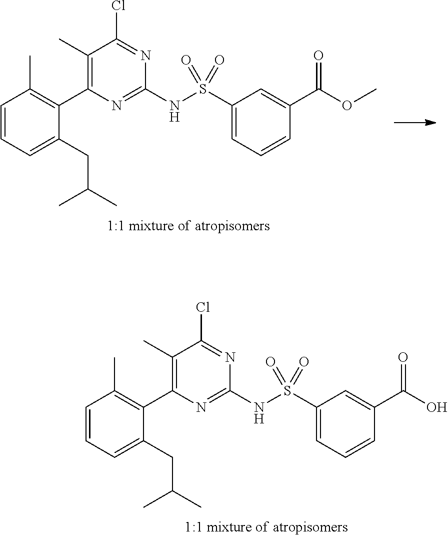 [SMILES: COC(=O)c1cccc(S(=O)(=O)Nc2nc(Cl)c(C)c(-c3c(C)cccc3CC(C)C)n2)c1 ; Cc1cccc(CC(C)C)c1-c1nc(NS(=O)(=O)c2cccc(C(=O)O)c2)nc(Cl)c1C]

To a stirring solution of methyl 3-[[4-chloro-6-(2-isobutyl-6-methyl-phenyl)-5-methyl-pyrimidin-2-yl]sulfamoyl]benzoate (3.787 g, 7.7603 mmol) in THE (65 mL) at room temperature was slowly added aqueous NaOH (40 mL of 1 M, 40.0 mmol). After the addition was complete, the reaction mixture was vigorously stirred at for 2 h. The reaction mixture was diluted with water (120 mL), and the volatiles were removed under vacuum. The residual aqueous layer was extracted with ethyl acetate (100 mL), and the organic layer was discarded. The aqueous layer was acidified with 2 M aqueous HCl to pH ˜1, and the product was extracted with ethyl acetate (250 mL). The aqueous layer was extracted with ethyl acetate (2×150 mL). The combined organic layers were evaporated to dryness to afford 3-[[4-chloro-6-(2-isobutyl-6-methyl-phenyl)-5-methyl-pyrimidin-2-yl]sulfamoyl]benzoic acid (3.54 g, 92%) as a white solid. ¹H NMR (500 MHz, DMSO-d6) δ 13.36 (broad s, 1H), 12.22 (broad s, 1H), 8.39 (t, J=1.9 Hz, 1H), 8.15 (d, J=7.7 Hz, 1H), 8.07 (d, J=7.8 Hz, 1H), 7.63 (t, J=7.8 Hz, 1H), 7.26 (t, J=7.6 Hz, 1H), 7.12 (d, J=7.5 Hz, 1H), 7.07 (d, J=7.6 Hz, 1H), 1.98-1.92 (m, 1H), 1.85-1.76 (m, 4H), 1.72 (s, 3H), 1.43-1.32 (m, 1H), 0.59 (d, J=6.5 Hz, 3H), 0.53 (d, J=6.5 Hz, 3H). ESI-MS m/z calc. 473.1176, found 474.1 (M+1)⁺; Retention time: 2.31 minutes; LC Method E.

### Step 9: 3-[[4-[(2R)-2-Amino-4,4-dimethyl-pentoxy]-6-(2-isobutyl-6-methyl-phenyl)-5-methyl-pyrimidin-2-yl]sulfamoyl]benzoic acid

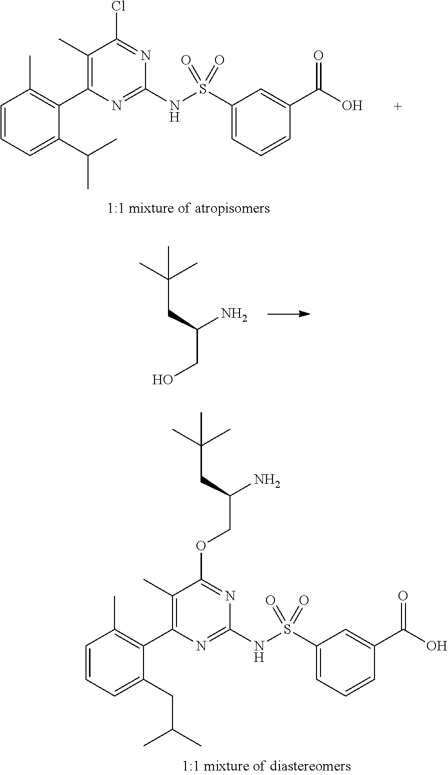

To a stirring suspension of 3-[[4-chloro-6-(2-isobutyl-6-methyl-phenyl)-5-methyl-pyrimidin-2-yl]sulfamoyl]benzoic acid (2.41 g, 5.0847 mmol) and (2R)-2-amino-4,4-dimethyl-pentan-1-ol (hydrochloride salt) (1.249 g, 7.4489 mmol) in anhydrous THE (80 mL) at room temperature under nitrogen was added sodium tert-butoxide (2.987 g, 31.081 mmol). Within 5-10 minutes, the reaction mixture became a homogeneous solution and the mixture was stirred for 2 h. The reaction mixture was cooled to 0° C., and slowly quenched with 1 N aqueous HCl (120 mL). The reaction mixture was allowed to warm to room temperature and stirred for 10 min. Ethyl acetate (150 mL) was added, and the reaction mixture was vigorously stirred for 15 min. The layers were separated, and the aqueous layer was extracted with ethyl acetate (2×100 mL). The combined organic layers were washed with brine (80 mL), dried over anhydrous sodium sulfate and concentrated. The crude was purified by reverse-phase HPLC using 40-80% acetonitrile gradient in water (5 mM HCl buffer) to afford 3-[[4-[(2R)-2-amino-4,4-dimethyl-pentoxy]-6-(2-isobutyl-6-methyl-phenyl)-5-methyl-pyrimidin-2-yl]sulfamoyl]benzoic acid (hydrochloride salt) (2.366 g, 74%) as a white solid. ¹H NMR (500 MHz, DMSO-d6) δ 8.47-8.40 (m, 1H), 8.17-8.03 (m, 3H), 7.66 (t, J=7.8 Hz, 1H), 7.29 (broad s, 1H), 7.22-7.06 (m, 2H), 4.29 (broad s, 1H), 4.12 (broad s, 1H), 3.59 (s, 1H), 2.54 (s, 2H), 2.20-1.77 (broad m, 5H), 1.73-1.47 (m, 6H), 0.92 (d, J=10.5 Hz, 9H), 0.68 (broad s, 6H). ESI-MS m/z calc. 568.2719, found 569.5 (M+1)⁺; Retention time: 2.15 minutes; LC Method E.

### Example 32: Preparation of (5P)-3-[[4-[(2R)-2-amino-4,4-dimethyl-pentoxy]-6-(2-isobutyl-6-methyl-phenyl)-5-methyl-pyrimidin-2-yl]sulfamoyl]benzoic acid

### Step 1: (5P)-4-chloro-5-methyl-6-[2-methyl-6-(2-methylpropyl)phenyl]pyrimidin-2-amine and (5M)-4-chloro-5-methyl-6-[2-methyl-6-(2-methylpropyl)phenyl]pyrimidin-2-amine

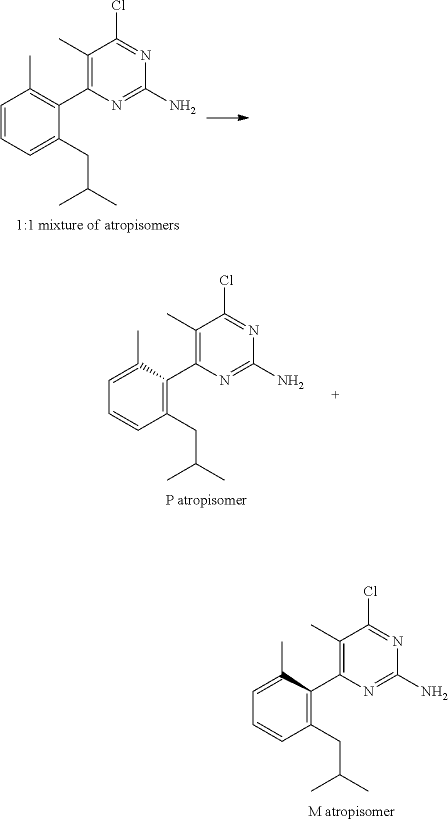

Racemic 4-chloro-6-(2-isobutyl-6-methyl-phenyl)-5-methyl-pyrimidin-2-amine (13.91 g, 48.00 mmol) was subjected to chiral SFC separation using the following conditions: IC column (3×25 cm), eluant: 10% MeOH/90% CO₂, 100 bar, flow rate: 80 mL/min, 220 nm, injection volume 0.5 mL, 5 mg/mL in methanol:DCM. Two atropisomers were isolated. SFC peak 1: (5P)-4-chloro-5-methyl-6-[2-methyl-6-(2-methylpropyl)phenyl]pyrimidin-2-amine (6.12 g, 88%). ESI-MS m/z calc. 289.13458, found 290.31 (M+1); Retention time: 1.84 minutes; LC Method A. SFC peak 2: (5M)-4-chloro-5-methyl-6-[2-methyl-6-(2-methylpropyl)phenyl]pyrimidin-2-amine (6.14 g, 88%). ESI-MS m/z calc. 289.13458, found 290.31 (M+1)⁺; Retention time: 1.85 minutes; LC Method A.

### Step 2: (5P)-Methyl 3-[[4-chloro-6-(2-isobutyl-6-methyl-phenyl)-5-methyl-pyrimidin-2-yl]sulfamoyl]benzoate

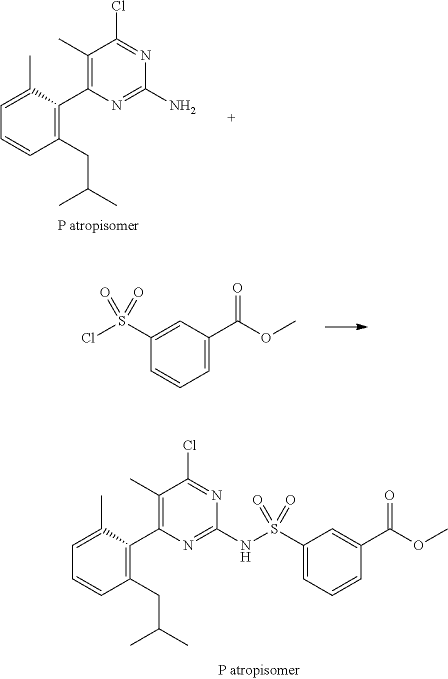

To a solution of 4-chloro-6-(2-isobutyl-6-methyl-phenyl)-5-methyl-pyrimidin-2-amine (6 g, 20.704 mmol) and methyl 3-chlorosulfonylbenzoate (14.6 g, 62.218 mmol) in THF (120 mL) was added a solution of LiHMDS in THE (83 mL of 1.0 M, 83.0 mmol) dropwise over 20 min at −78° C. The reaction mixture was stirred for 2 h and then quenched with 1 N HCl solution (100 mL). The layers were separated and the aqueous layer was extracted with ethyl acetate (2×250 mL). The combined organic layers were washed with brine, dried over sodium sulfate, filtered and concentrated in vacuo. The resulting yellow oil was purified silica gel chromatography (120 g silica, eluted with 10% ethyl acetate in hexanes) to give methyl 3-[[4-chloro-6-(2-isobutyl-6-methyl-phenyl)-5-methyl-pyrimidin-2-yl]sulfamoyl]benzoate (9.6 g, 86%) as a yellow sticky solid. ESI-MS m/z calc. 487.13324, found 488.8 (M+1)⁺; Retention time: 7.18 minutes; LC Method D.

### Step 3: (5P)-3-[[4-Chloro-6-(2-isobutyl-6-methyl-phenyl)-5-methyl-pyrimidin-2-yl]sulfamoyl]benzoic acid

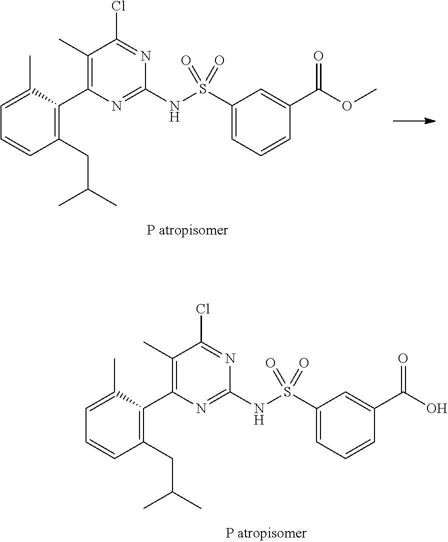

To a stirred solution of (5P)-methyl 3-[[4-chloro-6-(2-isobutyl-6-methyl-phenyl)-5-methyl-pyrimidin-2-yl]sulfamoyl]benzoate (9.6 g, 18.689 mmol) in THE (100 mL) was added aqueous NaOH solution (100 mL of 1 M, 100.0 mmol) at rt and stirred for 2 h. The reaction mixture was extracted with ethyl acetate (2×250 mL). The aqueous layer was acidified with 1 N HCl solution to pH=1 and then extracted with ethyl acetate (2×250 mL). The combined organic layer was washed with brine, dried over sodium sulfate, filtered and concentrated in vacuo to give 3-[[4-chloro-6-(2-isobutyl-6-methyl-phenyl)-5-methyl-pyrimidin-2-yl]sulfamoyl]benzoic acid (8.74 g, 97%) as a white solid. ESI-MS m/z calc. 473.1176, found 474.4 (M+1)⁺; Retention time: 6.69 minutes; LC Method DS.

### Step 4: (5P)-3-[[4-[(2R)-2-amino-4,4-dimethyl-pentoxy]-6-(2-isobutyl-6-methyl-phenyl)-5-methyl-pyrimidin-2-yl]sulfamoyl]benzoic acid

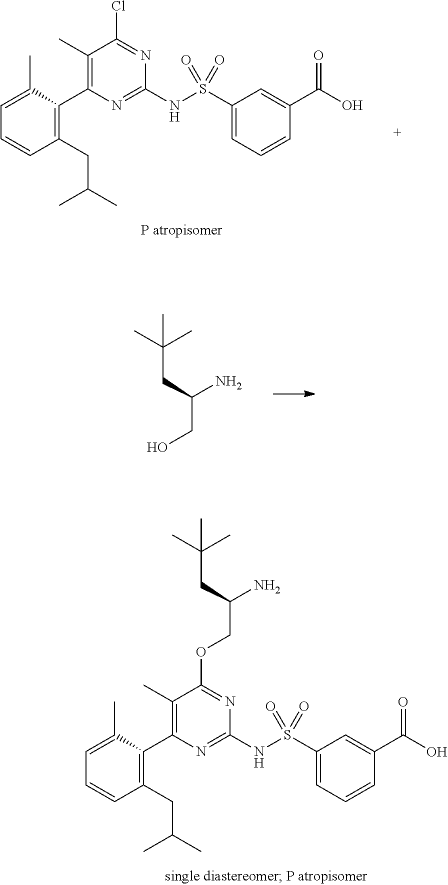

A solution of (5P)-3-[[4-chloro-6-(2-isobutyl-6-methyl-phenyl)-5-methyl-pyrimidin-2-yl]sulfamoyl]benzoic acid (7 g, 14.473 mmol), (2R)-2-amino-4,4-dimethyl-pentan-1-ol (hydrochloride salt) (6 g, 35.783 mmol) and sodium t-butoxide (7.2 g, 74.919 mmol) in THE (140.00 mL) was stirred at rt for 2 h. More (2R)-2-amino-4,4-dimethyl-pentan-1-ol (2.9 g, 22.101 mmol) and sodium t-butoxide (4.0 g, 41.622 mmol) were added and the reaction mixture was stirred for another hour. The reaction mixture was quenched with 1 M HCl and extracted with ethyl acetate (3×250 mL). The combined organic layer was washed with brine, dried over sodium sulfate, filtered and concentrated in vacuo to give (5P)-3-[[4-[(2R)-2-amino-4,4-dimethyl-pentoxy]-6-(2-isobutyl-6-methyl-phenyl)-5-methyl-pyrimidin-2-yl]sulfamoyl]benzoic acid (hydrochloride salt) (8.6 g, 88%) as a white solid. ESI-MS m/z calc. 568.2719, found 569.5 (M+1)⁺; Retention time: 2.07 minutes. ¹H NMR (500 MHz, DMSO-d6) δ 13.81 (bs, 1H), 8.42 (s, 1H), 8.28-8.17 (m, 3H), 8.10 (dd, J=14.3, 7.6 Hz, 2H), 7.66 (m, 1H), 7.33-7.21 (m, 1H), 7.13 (dd, J=14.1, 7.6 Hz, 2H), 4.27 (d, J=11.9 Hz, 1H), 4.11 (dd, J=12.3, 6.2 Hz, 1H), 3.58 (m, 1H), 2.17-1.95 (m, 2H), 1.89 (bs, 3H), 1.66 (s, 3H), 1.65-1.49 (m, 2H), 1.24 (m, 1H), 0.92 (s, 9H), 0.76-0.57 (m, 6H). LC method H.

### Example 33: Preparation of 3-[[4-[(2R)-2-amino-4,4-dimethyl-pentoxy]-6-[2-(2,2-dimethylpropyl)-6-methyl-phenyl]-5-methyl-pyrimidin-2-yl]sulfamoyl]benzoic acid

### Step 1: 2-Bromo-1-(2,2-dimethylpropyl)-3-methyl-benzene

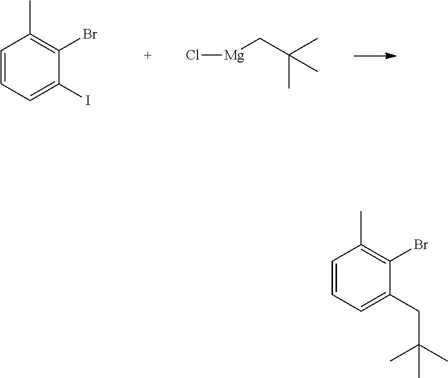

To a stirred solution of 2-bromo-1-iodo-3-methyl-benzene (10 g, 33.678 mmol) in dry dioxane (300 mL) at room temperature under nitrogen atmosphere were successively added Pd(amphos)Cl₂ (2.4 g, 3.3799 mmol) and a solution of chloro(2,2-dimethylpropyl)magnesium in tetrahydrofuran (44 mL of 1 M, 44.0 mmol). The reaction was stirred overnight at 30° C. Additional Pd(amphos)Cl₂ (1.2 g, 1.6899 mmol) and chloro(2,2-dimethylpropyl)magnesium in THE (22 mL of 1 M, 22.000 mmol) were added and the reaction was stirred overnight at 30° C. Then, aqueous 6 N HCl solution (250 mL) was added and the aqueous layer was extracted with pentane (5×250 mL). The combined organic layers were washed with water (500 mL), brine (500 mL) and dried over sodium sulfate. The organic layer then filtered on a pad of silica gel and washed with pentane (3×100 mL). The filtrate was concentrated under reduced pressure to afford crude 2-bromo-1-(2,2-dimethylpropyl)-3-methyl-benzene (11.06 g, 57%) as a pale brown oil.

### Step 2: [2-(2,2-Dimethylpropyl)-6-methyl-phenyl]boronic acid

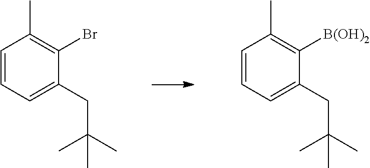

To a stirred solution of 2-bromo-1-(2,2-dimethylpropyl)-3-methyl-benzene (5.35 g, 22.184 mmol) in dry THE (350 mL) under nitrogen atmosphere cooled to −78° C. was added a solution of n-butyllithium in hexanes (10.7 mL of 2.5 M, 26.75 mmol). The reaction was stirred for 1 h at −78° C., after which trimethylborate (3.0030 g, 3.5 mL, 28.899 mmol) was added. The reaction was stirred for 45 min at −78° C. then allowed to warm to room temperature, then stirred at room temperature for 45 min. An aqueous solution of 1 N HCl (150 mL) was added and the reaction was vigorously stirred at room temperature for 30 min. The aqueous layer was extracted with dichloromethane (4×150 mL) and the combined extracts were washed with water (250 mL), brine (250 mL), dried over sodium sulfate, filtered and concentrated under reduced pressure to afford a pale yellow oil. This material was triturated with heptane (100 mL), filtered and dried under high vacuum to afford [2-(2,2-dimethylpropyl)-6-methyl-phenyl]boronic acid (2.253 g, 49%) as a white solid. ¹H NMR (400 MHz, DMSO-d6) δ 8.01 (s, 2H), 7.08 (t, J=6.8 Hz, 1H), 6.94 (d, J=7.3 Hz, 1H), 6.90 (d, J=7.3 Hz, 1H), 2.54 (s, 2H), 2.29 (s, 3H), 0.88 (s, 9H). ESI-MS m/z calc. 206.14781, found 205.3 (M−1)⁻; Retention time: 3.1 minutes; LC Method K.

### Step 3: tert-Butyl N-tert-butoxycarbonyl-N-[4-chloro-6-[2-(2,2-dimethylpropyl)-6-methyl-phenyl]-5-methyl-pyrimidin-2-yl]carbamate

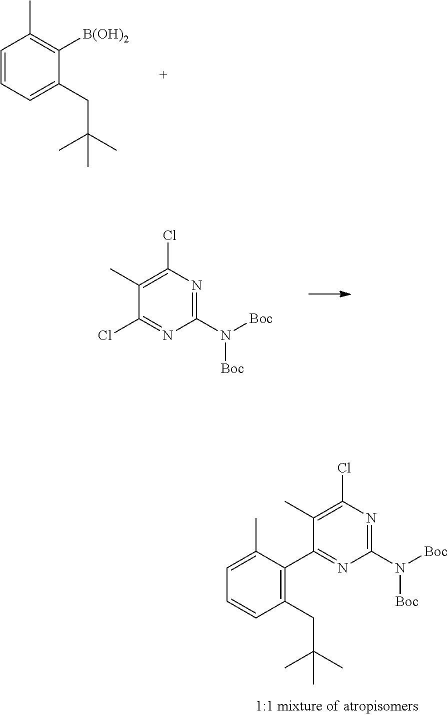 [SMILES: Cc1cccc(CC(C)(C)C)c1B(O)O ; Cc1c(Cl)nc(N(C(=O)OC(C)(C)C)C(=O)OC(C)(C)C)nc1Cl ; Cc1cccc(CC(C)(C)C)c1-c1nc(N(C(=O)OC(C)(C)C)C(=O)OC(C)(C)C)nc(Cl)c1C]

A reaction vial was charged with [2-(2,2-dimethylpropyl)-6-methyl-phenyl]boronic acid (1.09 g, 5.2890 mmol), tert-butyl N-tert-butoxycarbonyl-N-(4,6-dichloro-5-methyl-pyrimidin-2-yl)carbamate (2.412 g, 6.3767 mmol) and cesium carbonate (5.125 g, 15.730 mmol) in a solvent mixture of dimethoxyethane (9 mL) and water (3 mL). The reaction mixture was purged with argon for 3 min. Pd(dppf)Cl₂ (393 mg, 0.5371 mmol) was added to the reaction mixture. The reaction was stirred for 4 h. The reaction was diluted with ethyl acetate (30 mL) and washed with brine (20 mL). The organic layer was dried over anhydrous sodium sulfate and concentrated under vacuum. The residue was purified by reverse-phase HPLC using 80-100% acetonitrile in water (buffered with 0.1% TFA) to furnish tert-butyl N-tert-butoxycarbonyl-N-[4-chloro-6-[2-(2,2-dimethylpropyl)-6-methyl-phenyl]-5-methyl-pyrimidin-2-yl]carbamate (0.598 g, 21%). ESI-MS m/z calc. 503.2551, found 504.4 (M+1)⁺; Retention time: 4.58 minutes; LC Method E.

### Step 4: 4-Chloro-6-[2-(2,2-dimethylpropyl)-6-methyl-phenyl]-5-methyl-pyrimidin-2-amine

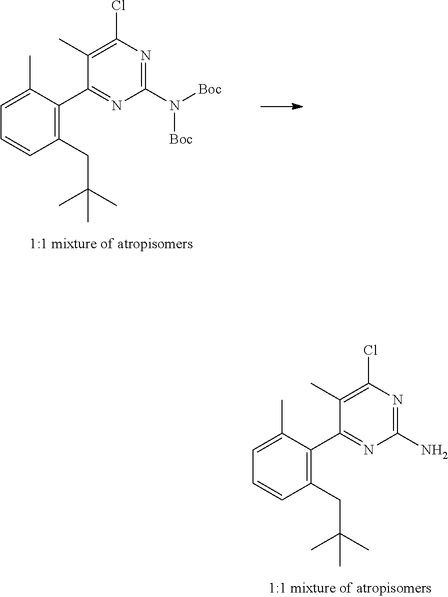 [SMILES: Cc1cccc(CC(C)(C)C)c1-c1nc(N(C(=O)OC(C)(C)C)C(=O)OC(C)(C)C)nc(Cl)c1C ; Cc1cccc(CC(C)(C)C)c1-c1nc(N)nc(Cl)c1C]

To a solution of tert-butyl N-tert-butoxycarbonyl-N-[4-chloro-6-[2-(2,2-dimethylpropyl)-6-methyl-phenyl]-5-methyl-pyrimidin-2-yl]carbamate (1.142 g, 2.2656 mmol) in anhydrous DCM (12 mL) was added 2.0 N HCl in ether (12 mL of 2 M, 24.0 mmol). The reaction was stirred at rt for 2 days. The solvents were removed under vacuum. The residue was diluted with DCM (50 mL) and washed with saturated sodium bicarbonate (30 mL), and brine (30 mL). The DCM layer was dried over anhydrous sodium sulfate and concentrated under vacuum to furnish 4-chloro-6-[2-(2,2-dimethylpropyl)-6-methyl-phenyl]-5-methyl-pyrimidin-2-amine (693 mg, 96%) as a white solid. ESI-MS m/z calc. 303.15024, found 304.2 (M+1)⁺; Retention time: 3.57 minutes; LC Method E.

### Step 5: Methyl 3-[[4-chloro-6-[2-(2,2-dimethylpropyl)-6-methyl-phenyl]-5-methyl-pyrimidin-2-yl]sulfamoyl]benzoate

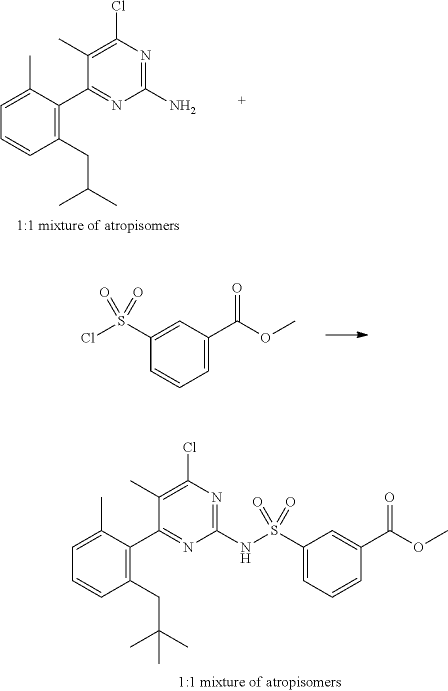 [SMILES: Cc1cccc(CC(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3CC(C)(C)C)n2)c1]

To a solution of 4-chloro-6-[2-(2,2-dimethylpropyl)-6-methyl-phenyl]-5-methyl-pyrimidin-2-amine (693 mg, 2.1782 mmol) and methyl 3-chlorosulfonylbenzoate (1.552 g, 6.6139 mmol) in anhydrous THE (12 mL) was added LiHMDS in THE (6.7 mL of 1.3 M, 8.71 mmol) dropwise at −78° C. The reaction was slowly warmed to rt and stirred for 2 h. The reaction was quenched with 10% aqueous citric acid (30 mL) and extracted with ethyl acetate (3×30 mL). The combined organic layers were washed with brine (30 mL), dried over anhydrous sodium sulfate and concentrated under vacuum. The residue was purified by silica gel chromatography using 0 to 20% acetone in hexane to furnish methyl 3-[[4-chloro-6-[2-(2,2-dimethylpropyl)-6-methyl-phenyl]-5-methyl-pyrimidin-2-yl]sulfamoyl]benzoate (1.084 g, 97%) as a white solid. ESI-MS m/z calc. 501.1489, found 502.1 (M+1)⁺; Retention time: 4.02 minutes; LC Method E.

### Step 6: 3-[[4-Chloro-6-[2-(2,2-dimethylpropyl)-6-methyl-phenyl]-5-methyl-pyrimidin-2-yl]sulfamoyl]benzoic acid

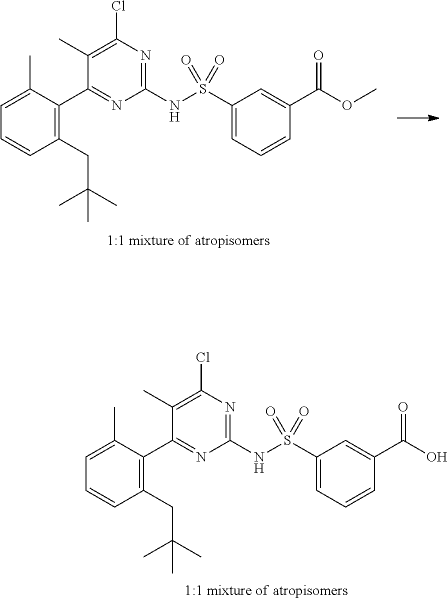 [SMILES: COC(=O)c1cccc(S(=O)(=O)Nc2nc(Cl)c(C)c(-c3c(C)cccc3CC(C)(C)C)n2)c1 ; Cc1cccc(CC(C)(C)C)c1-c1nc(NS(=O)(=O)c2cccc(C(=O)O)c2)nc(Cl)c1C]

To a solution of methyl 3-[[4-chloro-6-[2-(2,2-dimethylpropyl)-6-methyl-phenyl]-5-methyl-pyrimidin-2-yl]sulfamoyl]benzoate (1.084 g, 2.1161 mmol) in THE (11 mL) was added 1 N NaOH solution (11 mL, 11.0 mmol). The reaction was stirred at rt for 2 h. The reaction was diluted with 1 N HCl solution (30 mL) and TBME (30 mL). The layers were separated and the aqueous layer was extracted with TBME (2×30 mL). The combined organic layers were washed with brine (30 mL), dried over anhydrous magnesium sulfate and concentrated under vacuum to furnish 3-[[4-chloro-6-[2-(2,2-dimethylpropyl)-6-methyl-phenyl]-5-methyl-pyrimidin-2-yl]sulfamoyl]benzoic acid (1.076 g, 102%) as a white solid. ESI-MS m/z calc. 487.13324, found 488.1 (M+1)⁺; Retention time: 3.59 minutes; LC Method E.

### Step 7: 3-[[4-[(2R)-2-Amino-4,4-dimethyl-pentoxy]-6-[2-(2,2-dimethylpropyl)-6-methyl-phenyl]-5-methyl-pyrimidin-2-yl]sulfamoyl]benzoic acid

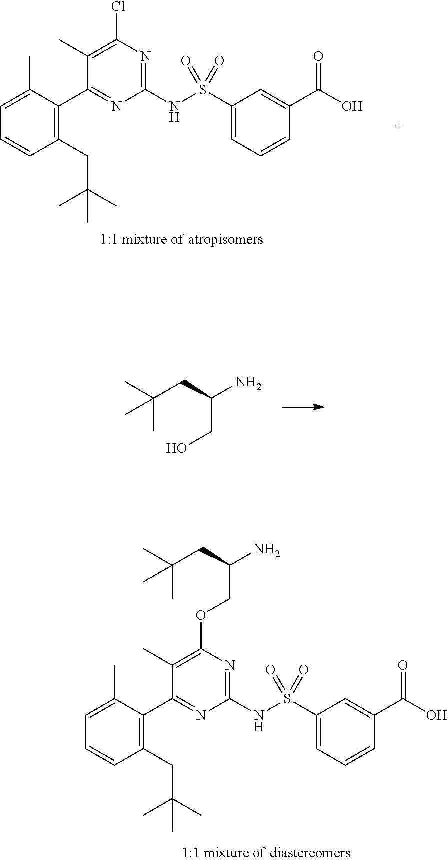

To a solution of 3-[[4-chloro-6-[2-(2,2-dimethylpropyl)-6-methyl-phenyl]-5-methyl-pyrimidin-2-yl]sulfamoyl]benzoic acid (1.076 g, 2.1608 mmol) and (2R)-2-amino-4,4-dimethyl-pentan-1-ol (hydrochloride salt) (561 mg, 3.3457 mmol) in anhydrous THE (20 mL) was added sodium tert-butoxide (1.047 g, 10.895 mmol). The reaction was stirred at rt for 2 h. Another portion of (2R)-2-amino-4,4-dimethyl-pentan-1-ol (hydrochloride salt) (147 mg, 0.8767 mmol) and sodium tert-butoxide (203 mg, 2.1123 mmol) was added. The reaction was stirred for 1 h, before it was quenched with 1 N HCl solution (30 mL). The reaction was extracted with ethyl acetate (3×30 mL). The combined organic layers were washed with brine (30 mL), dried over anhydrous magnesium sulfate and concentrated under vacuum. The residue was triturated with diethyl ether (10 mL) to furnish 3-[[4-[(2R)-2-amino-4,4-dimethyl-pentoxy]-6-[2-(2,2-dimethylpropyl)-6-methyl-phenyl]-5-methyl-pyrimidin-2-yl]sulfamoyl]benzoic acid (hydrochloride salt) (1.1193 g, 80%) as a white solid. ¹H NMR (500 MHz, DMSO-d6) δ 8.44-8.38 (m, 1H), 8.16 (s, 3H), 8.09 (m, 2H), 7.66 (t, J=7.8 Hz, 1H), 7.35-7.23 (m, 1H), 7.23-7.08 (m, 2H), 4.35-4.21 (m, 1H), 4.17-4.05 (m, 1H), 3.65-3.52 (m, 1H), 2.39-2.26 (m, 1H), 2.15-2.02 (m, 1H), 2.02-1.81 (m, 3H), 1.72-1.63 (m, 3H), 1.62-1.48 (m, 2H), 0.95-0.88 (m, 9H), 0.79-0.57 (m, 9H). ESI-MS m/z calc. 582.2876, found 583.3 (M+1)⁺; Retention time: 2.19 minutes; LC Method H.

### Example 34: Preparation of (5P)-3-[[4-[(2R)-2-amino-4,4-dimethyl-pentoxy]-6-(2-cyclopropyl-6-methyl-phenyl)-5-methyl-pyrimidin-2-yl]sulfamoyl]benzoic acid

### Step 1: 2-Cyclopropyl-6-methyl-phenol

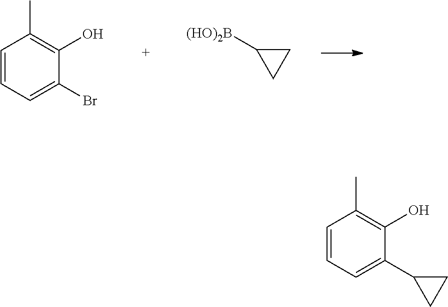

Palladium acetate (2.1 g, 9.3538 mmol) was added to 2-bromo-6-methyl-phenol (20 g, 106.93 mmol), cyclopropylboronic acid (13.8 g, 160.66 mmol), tricyclohexylphosphine (5 g, 17.830 mmol) and potassium phosphate (64.5 g, 303.86 mmol) in a suspension of toluene (640 mL) and water (160 mL). The reaction solution was refluxed for 6 h under nitrogen atmosphere. The reaction solution was cooled to room temperature, the organic phase was separated then washed with water (100 mL). The organic fraction was combined with heptane (˜300 mL) then filtered on a large pad of silica gel, then the silica gel was washed with a 1:1 heptane:DCM mixture. The fractions containing the desired product were combined and concentrated carefully (avoiding loss of the volatile desired product) to provide 2-cyclopropyl-6-methyl-phenol (17.7 g, 78%) as a brown oil. ¹H NMR (400 MHz, Chloroform-d) δ 7.02 (d, J=7.6 Hz, 1H), 6.97 (d, J=7.6 Hz, 1H), 6.78 (t, J=7.6 Hz, 1H), 5.56 (s, 1H), 2.28 (s, 3H), 1.79 (tt, J=8.3, 5.3 Hz, 1H), 1.04-0.94 (m, 2H), 0.69-0.62 (m, 2H).

### Step 2: (2-Cyclopropyl-6-methyl-phenyl)trifluoromethanesulfonate

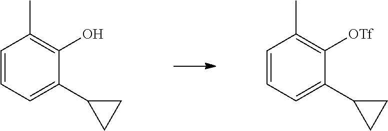 [SMILES: Cc1cccc(C2CC2)c1O ; Cc1cccc(C2CC2)c1OS(=O)(=O)C(F)(F)F]

Pyridine (15.648 g, 16 mL, 197.83 mmol) was added to a solution of 2-cyclopropyl-6-methyl-phenol (17.7 g, 119.43 mmol) in DCM (240 mL). The reaction mixture was cooled to 0° C., then trifluoromethylsulfonyl trifluoromethanesulfonate (43.602 g, 26 mL, 154.54 mmol) was slowly added. The reaction mixture was left to warm slowly to room temperature overnight. The reaction was diluted with DCM (200 mL) and the resulting mixture was washed with 0.2 N HCl (2×200 mL). The organic layer was washed with diluted NaHCO₃ (100 mL, 10× diluted saturated NaHCO₃ solution), then dried with sodium sulfate, filtered and concentrated in vacuo to provide (2-cyclopropyl-6-methyl-phenyl)trifluoromethanesulfonate (22.13 g, 66%) as a yellow oil. ¹H NMR (400 MHz, Chloroform-d) δ 7.17 (t, J=8.6 Hz, 1H), 7.10 (d, J=6.8 Hz, 1H), 6.86 (d, J=7.3 Hz, 1H), 2.40 (s, 3H), 2.18-2.07 (m, 1H), 1.11-1.02 (m, 2H), 0.77-0.68 (m, 2H). ¹⁹F NMR (377 MHz, Chloroform-d) 6-73.43 (s, 3F).

### Step 3: 2-(2-Cyclopropyl-6-methyl-phenyl)-4,4,5,5-tetramethyl-1,3,2-dioxaborolane

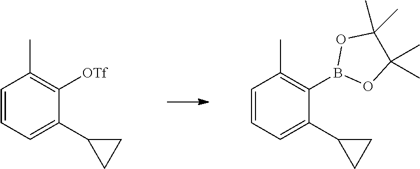 [SMILES: Cc1cccc(C2CC2)c1OS(=O)(=O)C(F)(F)F ; Cc1cccc(C2CC2)c1B1OC(C)(C)C(C)(C)O1]

A 500-mL round-bottomed flask was charged with molecular sieves 4 Å (7 g) and flame dried under vacuum. (2-Cyclopropyl-6-methyl-phenyl)trifluoromethanesulfonate (22 g, 78.498 mmol) then [1,1′-bis(diphenylphosphino)ferrocene]dichloropalladium(II) (2.8 g, 3.8267 mmol) were added. The flask was evacuated with nitrogen and then anhydrous dioxane (40 mL), triethyl amine (27.588 g, 38 mL, 272.64 mmol) and 4,4,5,5-tetramethyl-1,3,2-dioxaborolane (35.280 g, 40 mL, 275.67 mmol) were added. The reaction was refluxed for 4 h. The reaction mixture was cooled to room temperature, concentrated under reduced pressure, diluted with heptane and DCM, then purified by silica gel chromatography (with a gradient of 0 to 30% DCM in heptane) to provide 2-(2-cyclopropyl-6-methyl-phenyl)-4,4,5,5-tetramethyl-1,3,2-dioxaborolane (12.4 g, 60%) as a white solid. ¹H NMR (400 MHz, Chloroform-d) δ 7.13 (t, J=7.6 Hz, 1H), 6.94 (d, J=7.6 Hz, 1H), 6.74 (d, J=7.8 Hz, 1H), 2.40 (s, 3H), 2.11 (tt, J=8.5, 5.2 Hz, 1H), 1.41 (s, 12H), 0.93-0.81 (m, 2H), 0.74-0.64 (m, 2H). ESI-MS m/z calc. 258.1791, found 259.2 (M+1); Retention time: 4.98 minutes; LC Method J.

### Step 4: tert-Butyl N-tert-butoxycarbonyl-N-[4-chloro-6-(2-cyclopropyl-6-methyl-phenyl)-5-methyl-pyrimidin-2-yl]carbamate

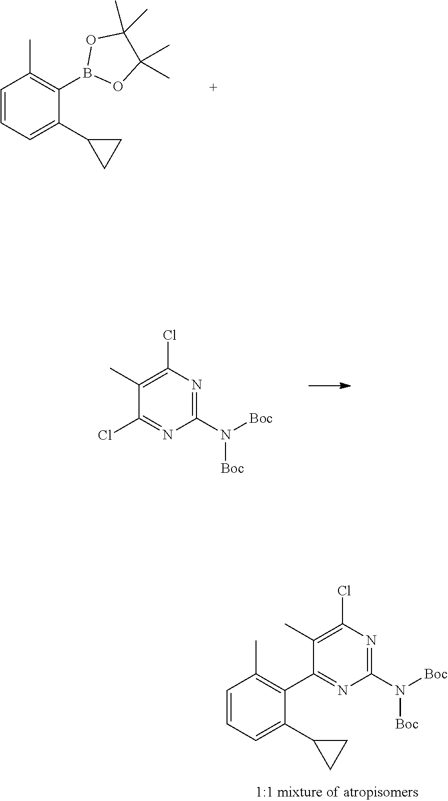 [SMILES: Cc1cccc(C2CC2)c1B1OC(C)(C)C(C)(C)O1 ; Cc1c(Cl)nc(N(C(=O)OC(C)(C)C)C(=O)OC(C)(C)C)nc1Cl ; Cc1cccc(C2CC2)c1-c1nc(N(C(=O)OC(C)(C)C)C(=O)OC(C)(C)C)nc(Cl)c1C]

A mixture of 2-(2-cyclopropyl-6-methyl-phenyl)-4,4,5,5-tetramethyl-1,3,2-dioxaborolane (1.50 g, 5.7231 mmol), tert-butyl N-tert-butoxycarbonyl-N-(4,6-dichloro-5-methyl-pyrimidin-2-yl)carbamate (2.03 g, 5.3668 mmol), and cesium carbonate (4.73 g, 14.517 mmol) in DME (30 mL) and water (30 mL) was purged with nitrogen for 15 min. Then, Pd(dppf)Cl₂ (422.7 mg, 0.5777 mmol) was added and the mixture was further purged with nitrogen for 5 min before stirring at 80° C. for 2 h. Upon cooling to room temperature, H₂O (30 mL) and EtOAc (30 mL) were added. The layers were separated and the aqueous layer was extracted with EtOAc (2×30 mL). The combined EtOAc layers were washed with saturated aqueous NaCl (30 mL), dried over anhydrous sodium sulfate, filtered and concentrated in vacuo. The crude product was subjected to silica gel chromatography (80 g SiO₂, eluting with 0 to 10% EtOAc/hexanes) to give a colorless oil, tert-butyl N-tert-butoxycarbonyl-N-[4-chloro-6-(2-cyclopropyl-6-methyl-phenyl)-5-methyl-pyrimidin-2-yl]carbamate (1.45 g, 51%); ESI-MS m z calc. 473.20813, found 474.1 (M+1)⁺; Retention time: 7.71 minutes; LC Method D.

### Step 5: 4-Chloro-6-(2-cyclopropyl-6-methyl-phenyl)-5-methyl-pyrimidin-2-amine

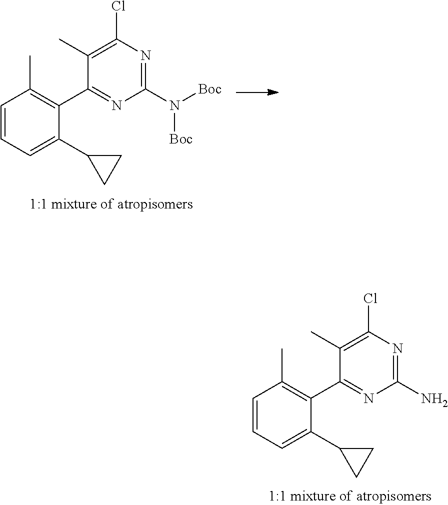 [SMILES: Cc1cccc(C2CC2)c1-c1nc(N(C(=O)OC(C)(C)C)C(=O)OC(C)(C)C)nc(Cl)c1C ; Cc1cccc(C2CC2)c1-c1nc(N)nc(Cl)c1C]

To a solution of tert-butyl N-tert-butoxycarbonyl-N-[4-chloro-6-(2-cyclopropyl-6-methyl-phenyl)-5-methyl-pyrimidin-2-yl]carbamate (1.45 g, 2.9062 mmol) in DCM (13 mL) stirring at 0° C. was added HCl in dioxane (20 mL of 4 M, 80.0 mmol). The reaction was then stirred at RT for 16 h. The reaction mixture was diluted with DCM (25 mL) and neutralized with saturated aqueous sodium bicarbonate (40 mL). The layers were separated and the aqueous layer was further extracted with DCM (2×25 mL). The combined DCM layers were washed with saturated aqueous NaCl (25 mL), dried over anhydrous sodium sulfate, filtered and concentrated under vacuum to give a white solid, 4-chloro-6-(2-cyclopropyl-6-methyl-phenyl)-5-methyl-pyrimidin-2-amine (952.4 mg, 109%); ¹H NMR (500 MHz, DMSO-d6) δ 7.21 (t, J=7.7 Hz, 1H), 7.08 (d, J=7.5 Hz, 1H), 6.96 (s, 2H), 6.77 (d, J=7.8 Hz, 1H), 1.96 (s, 3H), 1.82 (s, 3H), 1.47-1.35 (m, 1H), 0.84-0.72 (m, 2H), 0.72-0.62 (m, 1H), 0.56-0.43 (m, 1H). ESI-MS m z calc. 273.10327, found 274.0 (M+1)⁺; Retention time: 2.33 minutes; LC Method E.

### Step 6: (5P)-4-Chloro-6-(2-cyclopropyl-6-methylphenyl)-5-methylpyrimidin-2-amine and (5M)-4-chloro-6-(2-cyclopropyl-6-methylphenyl)-5-methylpyrimidin-2-amine

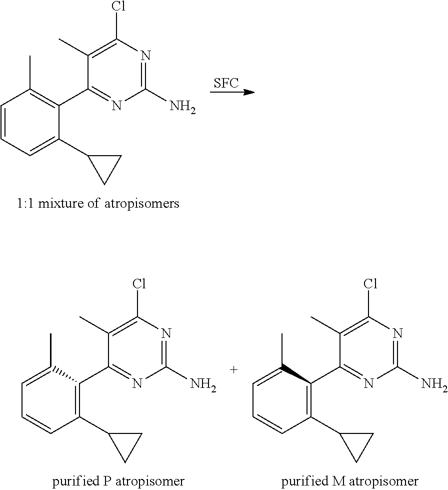

Racemic 4-chloro-6-(2-cyclopropyl-6-methyl-phenyl)-5-methyl-pyrimidin-2-amine (920 mg, 3.361 mmol) was subjected to chiral SFC separation using the following conditions: AD-H column (2×15 cm), eluant: 20% MeOH/CO₂, flow rate: 70 mL/min, concentration: 20 mg/mL in methanol:DCM, injection volume: 500 μL, pressure: 100 bar, wavelength: 220 nm. Two isomers were isolated. SFC peak 1: (5P)-4-chloro-6-(2-cyclopropyl-6-methylphenyl)-5-methylpyrimidin-2-amine (357 mg, 74%). ¹H NMR (400 MHz, DMSO-d6) δ 7.20 (t, J=7.7 Hz, 1H), 7.08 (dt, J=7.6, 1.0 Hz, 1H), 6.90 (broad s, 2H), 6.77 (d, J=7.8 Hz, 1H), 1.96 (s, 3H), 1.82 (s, 3H), 1.47-1.31 (m, 1H), 0.81-0.71 (m, 2H), 0.70-0.63 (m, 1H), 0.53-0.46 (m, 1H). ESI-MS m/z calc. 273.10327, found 274.17 (M+1)⁺; Retention time: 1.59 minutes; LC Method A. SFC peak 2: (5M)-4-chloro-6-(2-cyclopropyl-6-methylphenyl)-5-methylpyrimidin-2-amine (344 mg, 73%). ¹H NMR (400 MHz, DMSO-d6) δ 7.20 (t, J=7.7 Hz, 1H), 7.08 (dt, J=7.5, 1.0 Hz, 1H), 6.89 (broad s, 2H), 6.77 (d, J=7.8 Hz, 1H), 1.96 (s, 3H), 1.82 (s, 3H), 1.47-1.34 (m, 1H), 0.80-0.71 (m, 2H), 0.71-0.64 (m, 1H), 0.53-0.46 (m, 1H). ESI-MS m/z calc. 273.10327, found 274.17 (M+1)⁺; Retention time: 1.59 minutes; LC Method A.

### Step 7: (5P)-Methyl 3-[[4-chloro-6-(2-cyclopropyl-6-methyl-phenyl)-5-methyl-pyrimidin-2-yl]sulfamoyl]benzoate

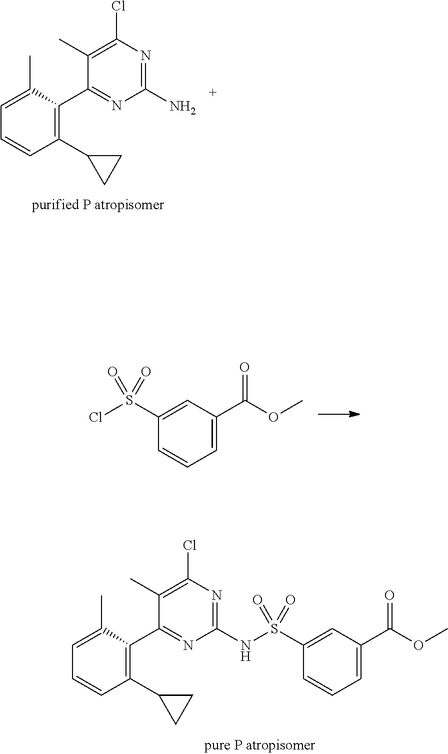

To a solution of (5P)-4-chloro-6-(2-cyclopropyl-6-methyl-phenyl)-5-methyl-pyrimidin-2-amine (300 mg, 1.0958 mmol) and methyl 3-chlorosulfonylbenzoate (770 mg, 3.2814 mmol) in THE (6.0 mL) was added a solution of LiHMDS in THE (4.2 mL of 1.0 M, 4.20 mmol) at −78° C. The reaction mixture was stirred for 2 h and then quenched with 1 N HCl solution (5 mL). The reaction mixture was warmed to room temperature, diluted with water (20 mL) and extracted with EtOAc (3×25 mL). The combined organic layers were washed with brine, dried over sodium sulfate, filtered and concentrated in vacuo. The resulting yellow solid was purified by silica gel chromatography (40 g silica, eluted with 20% ethyl acetate in hexanes) to give (5P)-methyl 3-[[4-chloro-6-(2-cyclopropyl-6-methyl-phenyl)-5-methyl-pyrimidin-2-yl]sulfamoyl]benzoate (370 mg, 71%) as a white solid. ESI-MS m/z calc. 471.10196, found 472.4 (M+1)⁺; Retention time: 6.69 minutes; LC Method D.

### Step 8: (5P)-3-[[4-Chloro-6-(2-cyclopropyl-6-methyl-phenyl)-5-methyl-pyrimidin-2-yl]sulfamoyl]benzoic acid

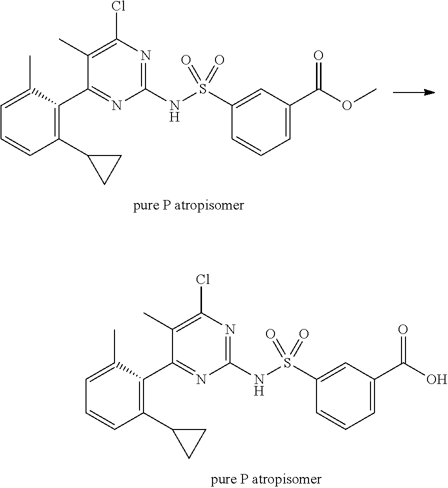

To a solution of (5P)-methyl 3-[[4-chloro-6-(2-cyclopropyl-6-methyl-phenyl)-5-methyl-pyrimidin-2-yl]sulfamoyl]benzoate (370 mg, 0.7761 mmol) in THE (7.4 mL) was added aqueous NaOH (4 mL of 1 M, 4.0 mmol) and stirred at rt for 2 h. The reaction mixture was diluted with water (25 mL) and extracted with ethyl acetate (50 mL). The aqueous layer was acidified to pH=1 and then extracted with ethyl acetate (50 mL). The organic layer was washed with brine, dried over sodium sulfate, filtered and concentrated in vacuo to give (5P)-3-[[4-chloro-6-(2-cyclopropyl-6-methyl-phenyl)-5-methyl-pyrimidin-2-yl]sulfamoyl]benzoic acid (352 mg, 97%) as a white solid. ESI-MS m/z calc. 457.0863, found 458.4 (M+1)⁺; Retention time: 6.38 minutes; LC Method D.

### Step 9: (5P)-3-[[4-[(2R)-2-Amino-4,4-dimethyl-pentoxy]-6-(2-cyclopropyl-6-methyl-phenyl)-5-methyl-pyrimidin-2-yl]sulfamoyl]benzoic acid

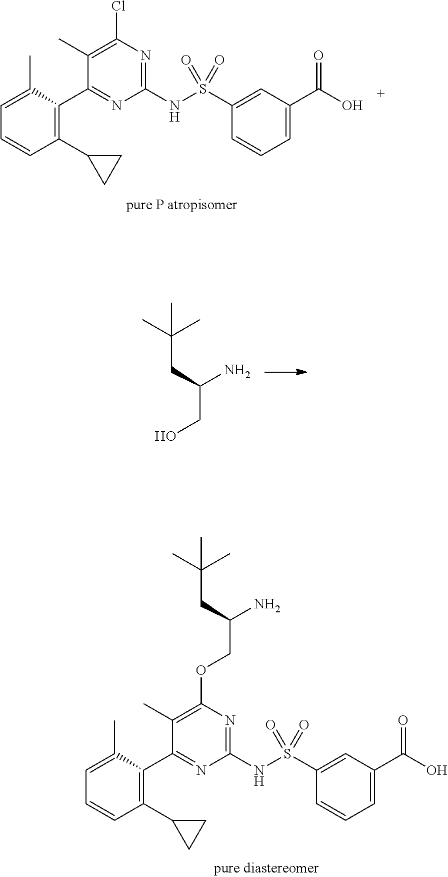

To a stirred solution of (5P)-3-[[4-chloro-6-(2-cyclopropyl-6-methyl-phenyl)-5-methyl-pyrimidin-2-yl]sulfamoyl]benzoic acid (350 mg, 0.7490 mmol) and (2R)-2-amino-4,4-dimethyl-pentan-1-ol (hydrochloride salt) (300 mg, 1.7892 mmol) in THE (7.0 mL) was added sodium t-butoxide (350 mg, 3.6419 mmol) at rt and stirred for 4 h. Another portion of (2R)-2-amino-4,4-dimethyl-pentan-1-ol (hydrochloride salt) (150 mg, 0.8946 mmol) and sodium t-butoxide (150 mg, 1.5608 mmol) was added and the reaction mixture was stirred for 1 h. The reaction mixture was cooled to 0° C. and quenched with 10 mL of 1 N HCl solution. The layers were separated and the aqueous layer was extracted with ethyl acetate (2×25 mL). The combined organic layers were washed with brine, dried over sodium sulfate, filtered and concentrated in vacuo. The resulting white solid was purified by reverse-phase HPLC (C₁₈ column, with a gradient of 30 to 75% acetonitrile in water, with 0.1% HCl as a modifier) to give (5P)-3-[[4-[(2R)-2-amino-4,4-dimethyl-pentoxy]-6-(2-cyclopropyl-6-methyl-phenyl)-5-methyl-pyrimidin-2-yl]sulfamoyl]benzoic acid (hydrochloride salt) (332 mg, 73%) as a white solid. ¹H NMR (500 MHz, DMSO-d6) δ 13.33 (s, 1H), 8.44 (s, 1H), 8.10 (dd, J=13.4, 7.7 Hz, 2H), 7.99 (bs, 3H), 7.66 (dd, J=7.8, 7.8 Hz, 1H), 7.26 (dd, J=7.7, 7.7 Hz, 1H), 7.11 (d, J=7.6 Hz, 1H), 6.81 (d, J=7.9 Hz, 1H), 4.24 (dd, J=12.1, 2.9 Hz, 1H), 4.07 (dd, J=12.1, 6.3 Hz, 1H), 3.62-3.55 (m, 1H), 1.91 (bs, 3H), 1.70 (s, 3H), 1.62 (dd, J=14.6, 7.2 Hz, 1H), 1.48 (dd, J=14.6, 3.9 Hz, 1H), 1.38-1.27 (m, 1H), 0.92 (s, 9H), 0.76-0.61 (m, 3H), 0.60-0.48 (m, 1H). ESI-MS m/z calc. 552.24066, found 553.3 (M+1)⁺; Retention time: 1.94 minutes; LC Method H.

### Example 35: Preparation of 3-[[4-[(2R)-2-amino-4,4-dimethyl-pentoxy]-6-(2-isopentyl-6-methyl-phenyl)-5-methyl-pyrimidin-2-yl]sulfamoyl]benzoic acid

### Step 1: 3-Methyl-2-(4, 4,5,5-tetramethyl-1,3,2-dioxaborolan-2-yl)benzaldehyde

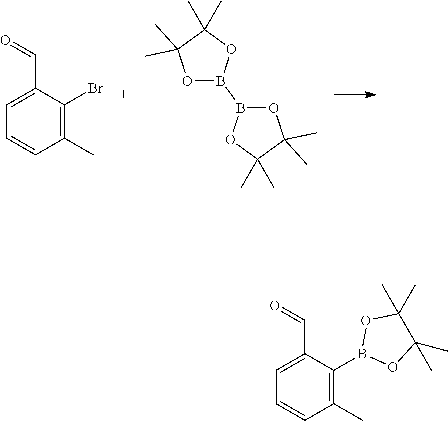

Prepared a solution of 2-bromo-3-methyl-benzaldehyde (22.5 g, 113.04 mmol), bis(pinacolato)diboron (43.1 g, 169.73 mmol), and KOAc (22.2 g, 226.20 mmol) in 1,4-dioxane (500 mL). The resulting slurry was sparged with a nitrogen stream for five minutes, then [1,1′-Bis(diphenylphosphino)ferrocene]dichloropalladium(II) (8.3 g, 11.343 mmol) was added and the mixture was refluxed under nitrogen for twenty hours, then cooled to room temperature and quenched with 1M hydrochloric acid until the pH was approximately 3-4. The phases were then separated: the aqueous phase was discarded and the organic phase was concentrated in vacuo, combined with a crude product from a previous batch run using 2.5 g of 2-bromo-3-methyl-benzaldehyde and purified by silica gel chromatography using 0 to 10% ethyl acetate in hexane to obtain 3-methyl-2-(4,4,5,5-tetramethyl-1,3,2-dioxaborolan-2-yl)benzaldehyde (22.5 g, 81%) as a pale yellow oil. ¹H NMR (500 MHz, Chloroform-d) δ 9.98 (s, 1H), 7.63 (dd, J=6.6, 2.1 Hz, 1H), 7.43 (d, J=6.6 Hz, 2H), 2.49 (s, 3H), 1.49 (s, 12H). ESI-MS m/z calc. 246.14273, found 247.2 (M+1)⁺; Retention time: 0.66 minutes; LC method D.

### Step 2: tert-Butyl N-tert-butoxycarbonyl-N-[4-chloro-6-(2-formyl-6-methyl-phenyl)-5-methyl-pyrimidin-2-yl]carbamate

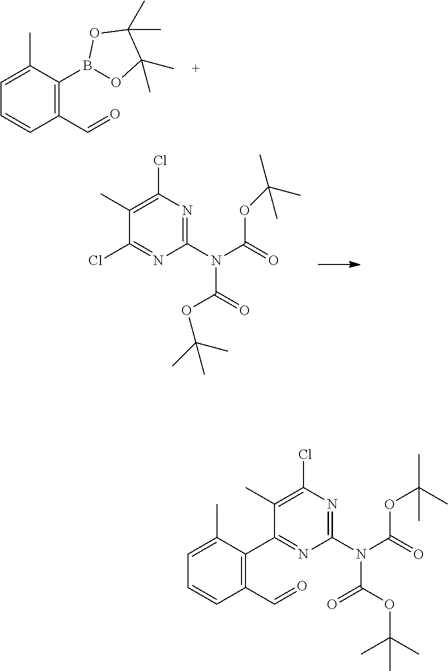

A flask was charged with 3-methyl-2-(4,4,5,5-tetramethyl-1,3,2-dioxaborolan-2-yl)benzaldehyde (19.2 g, 54.610 mmol), tert-butyl N-tert-butoxycarbonyl-N-(4,6-dichloro-5-methyl-pyrimidin-2-yl)carbamate (20 g, 49.702 mmol), cesium carbonate (46 g, 141.18 mmol) and Pd(dppf)Cl₂·DCM (4.06 g, 4.9716 mmol) under argon. A pre-degassed (bubbling argon for 1 h, both the solvents)dimethoxyethane (130 mL) and water (42 mL) were added to the reaction mixture. The reaction mixture was then further degassed with argon for 10 minutes. The mixture was then heated to 85° C. for 2 h. The reaction was then allowed to cool to RT and diluted with water (200 mL) and EtOAc (200 mL). The aqueous layer was extracted with EtOAc (3×200 mL). The organic layers were then combined, washed with brine (200 ml), dried with anhydrous sodium sulfate, filtered, and concentrated under reduced pressure. The crude product was purified by flash chromatography (Combiflash, loaded onto dry silica in 2×330 g cartridge using 0-40% diethyl ether in hexanes (1% Et₃N as modifier) as eluent) and the solvent was removed to provide tert-butyl N-tert-butoxycarbonyl-N-[4-chloro-6-(2-formyl-6-methyl-phenyl)-5-methyl-pyrimidin-2-yl]carbamate (9.2 g, 38%) as an off white solid. ¹H NMR (500 MHz, DMSO-d6) δ 9.79 (s, 1H), 7.94 (dd, J=7.5, 1.4 Hz, 1H), 7.76-7.66 (m, 2H), 2.02 (s, 3H), 2.00 (s, 3H), 1.36 (s, 18H). ESI-MS m/z calc. 461.17175, found 462.1 (M+1)⁺; Retention time: 3.15 minutes; LC method H.

### Step 3: tert-Butyl N-tert-butoxycarbonyl-N-[4-chloro-6-[2-(hydroxymethyl)-6-methyl-phenyl]-5-methyl-pyrimidin-2-yl]carbamate

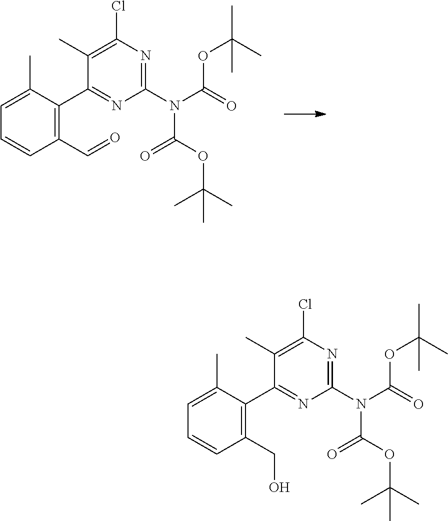 [SMILES: Cc1cccc(C=O)c1-c1nc(N(C(=O)OC(C)(C)C)C(=O)OC(C)(C)C)nc(Cl)c1C ; Cc1cccc(CO)c1-c1nc(N(C(=O)OC(C)(C)C)C(=O)OC(C)(C)C)nc(Cl)c1C]

tert-Butyl N-tert-butoxycarbonyl-N-[4-chloro-6-(2-formyl-6-methyl-phenyl)-5-methyl-pyrimidin-2-yl]carbamate (3 g, 5.8449 mmol) was dissolved in anhydrous THE (28.5 mL) and the solution was cooled to 0° C. Sodium borohydride (425 mg, 0.4497 mL, 11.234 mmol) was then added portionwise to it. The reaction was then stirred for 30 min at 0° C. The reaction was then warmed up to room temperature and quenched with water (50 ml) and saturated ammonium chloride solution (50 ml) and EtOAc (100 ml) was added to it. The reaction mixture was then stirred at room temperature for 10 min. The aqueous layer was extracted with EtOAc (3×100 ml) and the combined organic solution was washed with brine (70 ml), dried over anhydrous sodium sulfate, filtered and the solvent was removed under reduced pressure. The crude product was purified by flash chromatography (Combiflash, loaded onto dry silica in 80 g cartridge using 0-50% EtOAc in hexanes (1% Et₃N as modifier) as eluent to provide tert-butyl N-tert-butoxycarbonyl-N-[4-chloro-6-[2-(hydroxymethyl)-6-methyl-phenyl]-5-methyl-pyrimidin-2-yl]carbamate (2.51 g, 87%) as a colorless foamy solid. ESI-MS m/z calc. 463.1874, found 464.3 (M+1)⁺; Retention time: 6.15 minutes; LC method D.

### Step 4: tert-Butyl N-[4-[2-(bromomethyl)-6-methyl-phenyl]-6-chloro-5-methyl-pyrimidin-2-yl]-N-tert-butoxycarbonyl-carbamate

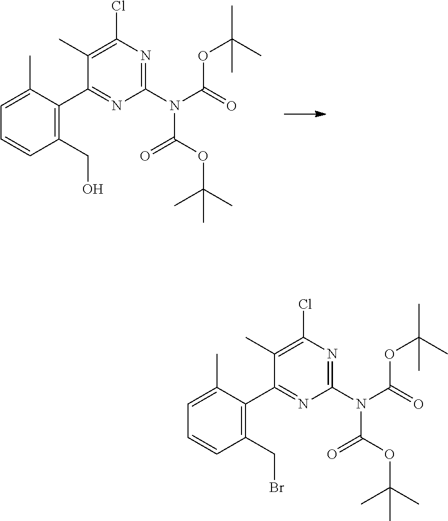 [SMILES: Cc1cccc(CO)c1-c1nc(N(C(=O)OC(C)(C)C)C(=O)OC(C)(C)C)nc(Cl)c1C ; Cc1cccc(CBr)c1-c1nc(N(C(=O)OC(C)(C)C)C(=O)OC(C)(C)C)nc(Cl)c1C]

tert-Butyl N-tert-butoxycarbonyl-N-[4-chloro-6-[2-(hydroxymethyl)-6-methyl-phenyl]-5-methyl-pyrimidin-2-yl]carbamate (7.5 g, 15.034 mmol) and carbon tetrabromide (7.5 g, 22.616 mmol) were dissolved in anhydrous dichloromethane (75 mL) and the solution was cooled to 0° C. A solution of triphenyl phosphine (6 g, 22.876 mmol) in anhydrous dichloromethane (23 mL) was added dropwise over 10 min. The reaction mixture was then stirred at the same temperature for for 45 min (NOTE). The solvent was removed by rotary evaporation. The crude product was purified by flash chromatography (Combiflash, loaded onto dry silica in 220 g cartridge using 0-30% diethyl ether in hexanes (1% Et₃N as modifier) as eluent and the solvent was removed to provide tert-butyl N-[4-[2-(bromomethyl)-6-methyl-phenyl]-6-chloro-5-methyl-pyrimidin-2-yl]-N-tert-butoxycarbonyl-carbamate (7.3 g, 83%) as white foamy solid. ESI-MS m/z calc. 525.10297, found 526.5 (M+1)⁺; Retention time: 7.88 minutes; LC method D.

### Step 5: [2-[2-[bis(tert-Butoxycarbonyl)amino]-6-chloro-5-methyl-pyrimidin-4-yl]-3-methyl-phenyl]methyl-triphenyl-phosphonium

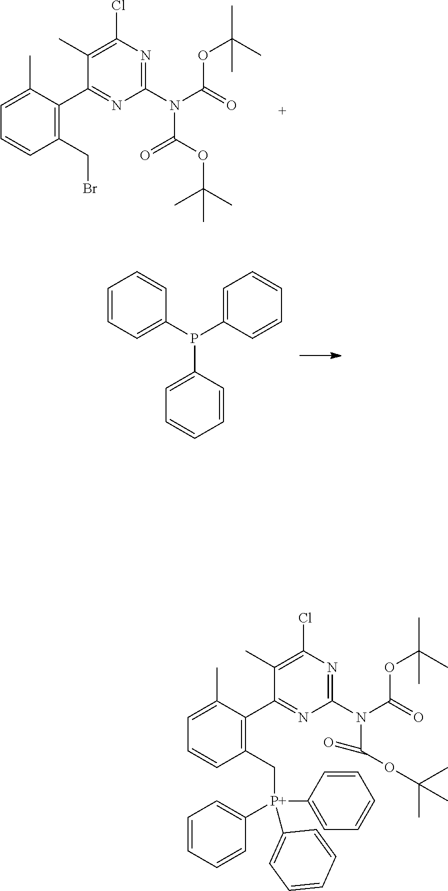 [SMILES: Cc1cccc(CBr)c1-c1nc(N(C(=O)OC(C)(C)C)C(=O)OC(C)(C)C)nc(Cl)c1C ; c1ccc(P(c2ccccc2)c2ccccc2)cc1 ; Cc1cccc(C[P+](c2ccccc2)(c2ccccc2)c2ccccc2)c1-c1nc(N(C(=O)OC(C)(C)C)C(=O)OC(C)(C)C)nc(Cl)c1C]

tert-Butyl N-[4-[2-(bromomethyl)-6-methyl-phenyl]-6-chloro-5-methyl-pyrimidin-2-yl]-N-tert-butoxycarbonyl-carbamate (3.15 g, 5.3810 mmol) was dissolved in anhydrous toluene (22 mL) at room temperature and the solution was degassed (bubbling argon) for 5 min. triphenylphosphane (1.6 g, 1.4134 mL, 6.1002 mmol) was then added to the solution and stirred for 5 min at room temperature under nitrogen. The reaction mixture was then heated at 85° C. for 5 h (white solid started crashing out after 30 min of heating which was indicated the formation of phosphonium salt) and the starting material was completely consumed at this time. The reaction mixture was then cooled to room temperature and white solid formed was then filtered through sintered funnel washing thoroughly with cold toluene (cooling in ice-water bath) to remove most of the organic impurities (excess triphenylphosphine, triphenylphosphine oxide) and the solid was dried under vacuum to give [2-[2-[bis(tert-butoxycarbonyl)amino]-6-chloro-5-methyl-pyrimidin-4-yl]-3-methyl-phenyl]methyl-triphenyl-phosphonium (Bromide Ion (1)) (3.8 g, 85%) as white solid. ¹H NMR (500 MHz, DMSO-d6) δ 7.90-7.83 (m, 3H), 7.70-7.62 (m, 6H), 7.55-7.43 (m, 6H), 7.39-7.33 (m, 1H), 7.23 (t, J=7.8 Hz, 1H), 6.78 (dd, J=7.9, 2.4 Hz, 1H), 4.99 (t, J=15.4 Hz, 1H), 4.48 (t, J=15.7 Hz, 1H), 1.88 (s, 3H), 1.76 (s, 3H), 1.39 (s, 18H). ESI-MS m/z calc. 708.2758, found 708.7 (M+)⁺; null (M−)⁺; Retention time: 3.02 minutes; LC method H.

### Step 6: tert-Butyl N-tert-butoxycarbonyl-N-[4-chloro-5-methyl-6-[2-methyl-6-[(E)-3-methylbut-1-enyl]phenyl]pyrimidin-2-yl]carbamate; tert-butyl N-tert-butoxycarbonyl-N-[4-chloro-5-methyl-6-[2-methyl-6-[(Z)-3-methylbut-1-enyl]phenyl]py

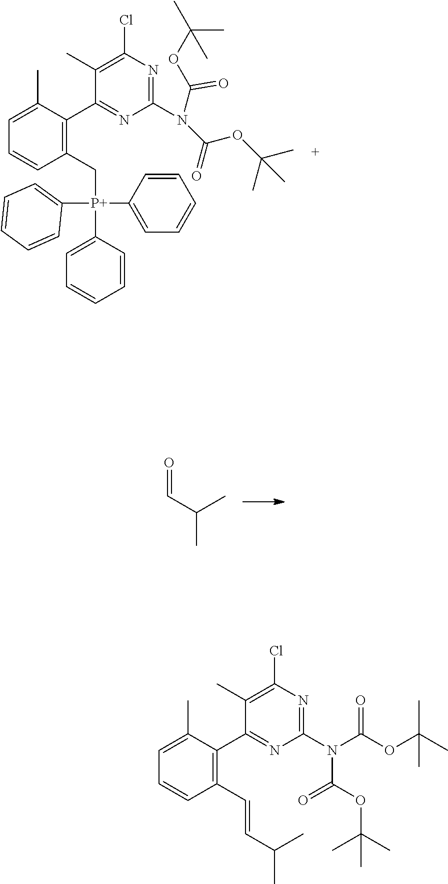

To a solution of [2-[2-[bis(tert-butoxycarbonyl)amino]-6-chloro-5-methyl-pyrimidin-4-yl]-3-methyl-phenyl]methyl-triphenyl-phosphonium (Bromide Ion (1)) (5 g, 6.3360 mmol) in DCM (20 mL) was added potassium carbonate (880 mg, 6.3673 mmol) and 18-Crown-6 (335 mg, 1.2674 mmol) at room temperature and the resulting orange solution was added 2-methylpropanal (922.50 mg, 0.750 mL, 12.794 mmol). The mixture was heated in a 45° C. oil bath for 3.5 h. The reaction was stirred at RT 72 h. The reaction was concentrated in vacuo, triturated in TBME (50 mL) at 55° C. for 30 min cooled to room temperature and filtered through packed celite, then rinsed filter with TBME (2×50 mL). The filtrate was concentrated in vacuo and the resulting residue was purified by flash chromatography (Combiflash, loaded onto [pre-equilibrated with hexanes-Et₃N (0.5%)]220 g SiO₂ cartridge with benzene, and eluted with 0-4% EtOAc in Hexanes over 90 minute gradient; no Et₃N used during elution) to provide tert-butyl N-tert-butoxycarbonyl-N-[4-chloro-5-methyl-6-[2-methyl-6-[(E)-3-methylbut-1-enyl]phenyl]pyrimidin-2-yl]carbamate; tert-butyl N-tert-butoxycarbonyl-N-[4-chloro-5-methyl-6-[2-methyl-6-[(Z)-3-methylbut-1-enyl]phenyl]py (2.5 g, 75%) as a colorless oil (major product E-isomer). ¹H NMR (500 MHz, Chloroform-d) δ 7.44-7.40 (m, 1H), 7.32-7.27 (m, 1H), 7.17-7.13 (m, 1H), 6.05 (dd, J=15.6, 7.5 Hz, 1H), 5.81 (d, J=15.5 Hz, 1H), 2.31-2.20 (m, 1H), 2.08 (s, 3H), 2.01 (s, 3H), 1.44 (s, 18H), 0.96 (dd, J=6.7, 2.3 Hz, 6H). ESI-MS m/z calc. 501.23944, found 502.5 (M+1)⁺; Retention time: 8.78 minutes; LC method D.

### Step 7: tert-Butyl N-tert-butoxycarbonyl-N-[4-chloro-6-(2-isopentyl-6-methyl-phenyl)-5-methyl-pyrimidin-2-yl]carbamate

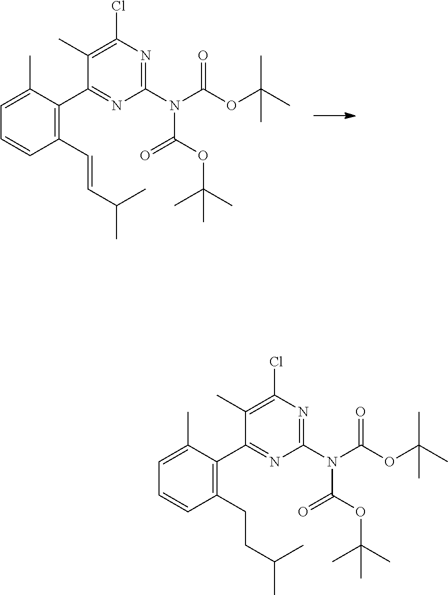 [SMILES: Cc1cccc(/C=C/C(C)C)c1-c1nc(N(C(=O)OC(C)(C)C)C(=O)OC(C)(C)C)nc(Cl)c1C ; Cc1cccc(CCC(C)C)c1-c1nc(N(C(=O)OC(C)(C)C)C(=O)OC(C)(C)C)nc(Cl)c1C]

To a solution of tert-butyl N-tert-butoxycarbonyl-N-[4-chloro-5-methyl-6-[2-methyl-6-[(E)-3-methylbut-1-enyl]phenyl]pyrimidin-2-yl]carbamate (2.5 g, 4.9796 mmol) in EtOAc (200 mL) was added Pd—C(475 mg, 10% w/w, 0.4463 mmol) under N₂. The reaction was cooled to 0° C. then bubbled H₂ gas through the reaction then placed under a H₂ balloon and stirred at room temperature. The reaction was stirred 4 h at 0° C. The reaction was purged with N₂ gas then filtered through packed celite and the filtrate concentrated in vacuo to provide tert-butyl N-tert-butoxycarbonyl-N-[4-chloro-6-(2-isopentyl-6-methyl-phenyl)-5-methyl-pyrimidin-2-yl]carbamate (2.48 g, 98%). ¹H NMR (500 MHz, Chloroform-d) δ 7.31-7.27 (m, 1H), 7.14 (dd, J=19.6, 7.6 Hz, 2H), 2.31-2.22 (m, 2H), 2.12 (s, 3H), 1.97 (s, 3H), 1.43 (s, 18H), 1.31-1.22 (m, 2H), 0.98-0.93 (m, 1H), 0.81 (dd, J=6.4, 1.3 Hz, 6H). ESI-MS m/z calc. 503.2551, found 504.5 (M+1)⁺; Retention time: 8.82 minutes; LC method D.

### Step 8: 4-Chloro-6-(2-isopentyl-6-methyl-phenyl)-5-methyl-pyrimidin-2-amine

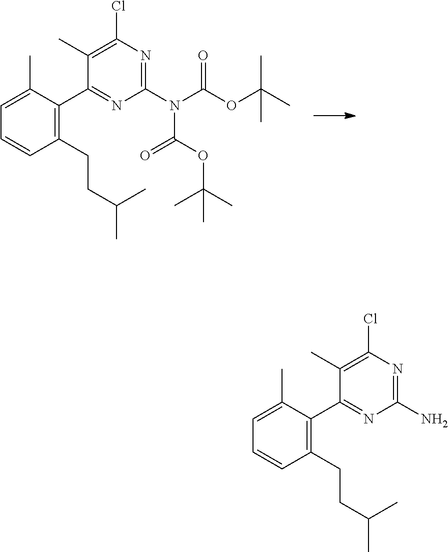 [SMILES: Cc1cccc(CCC(C)C)c1-c1nc(N(C(=O)OC(C)(C)C)C(=O)OC(C)(C)C)nc(Cl)c1C ; Cc1cccc(CCC(C)C)c1-c1nc(N)nc(Cl)c1C]

A solution of tert-butyl N-tert-butoxycarbonyl-N-[4-chloro-6-(2-isopentyl-6-methyl-phenyl)-5-methyl-pyrimidin-2-yl]carbamate (2.48 g, 4.9200 mmol) in HFIP (40 mL) split half evenly dispensed over 2 microwave vials and each was heated at 100° C. in a microwave reactor for 1 h. The contents of both vials were combined, and the reaction was concentrated in vacuo. The resulting residue was purified by flash chromatography (Combiflash, loaded onto an 80 g SiO₂ cartridge with benzene, and eluted with 0-5% EtOAc in Hexanes over a 45-minute gradient) to provide 4-chloro-6-(2-isopentyl-6-methyl-phenyl)-5-methyl-pyrimidin-2-amine (1.0659 g, 70%) (yield is over 2-steps) as a colorless oil. ¹H NMR (500 MHz, Chloroform-d) δ 7.23 (t, J=7.7 Hz, 1H), 7.14-7.06 (m, 2H), 5.13-5.01 (m, 2H), 2.34 (ddd, J=13.6, 10.6, 5.6 Hz, 1H), 2.22 (ddd, J=13.6, 10.9, 5.2 Hz, 1H), 2.03 (s, 3H), 1.93 (d, J=0.5 Hz, 3H), 1.48-1.35 (m, 2H), 1.33-1.24 (m, 1H), 0.84-0.73 (m, 6H). ESI-MS m/z calc. 303.15024, found 304.0 (M+1)⁺; Retention time: 2.99 minutes; LC method H.

### Step 9: Methyl 3-[[4-chloro-6-(2-isopentyl-6-methyl-phenyl)-5-methyl-pyrimidin-2-yl]sulfamoyl]benzoate

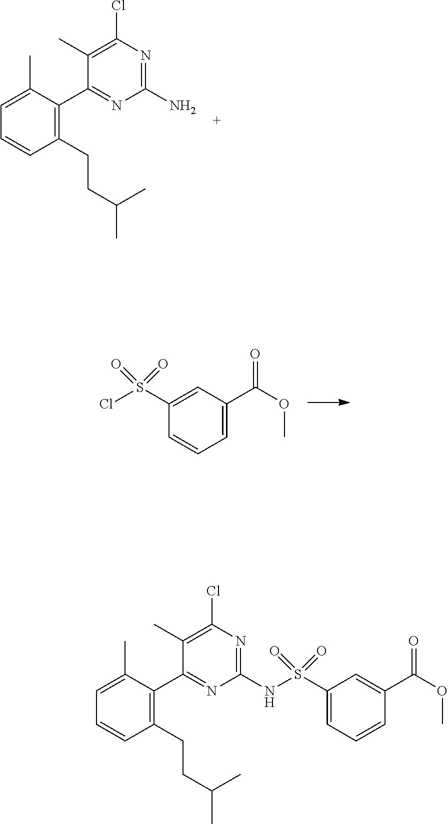

To a solution of 4-chloro-6-(2-isopentyl-6-methyl-phenyl)-5-methyl-pyrimidin-2-amine (768 mg, 2.4671 mmol) and methyl 3-chlorosulfonylbenzoate (1.451 g, 6.1835 mmol) in anhydrous THE (41 mL) at −78° C. under nitrogen was added LiHMDS in THE (7.6 mL of 1.3 M, 9.8800 mmol) dropwise. The mixture was stirred at −78° C. for 2 h. The reaction was quenched still cold with 2 N HCl aqueous solution (23 mL) and EtOAc (20 mL). the mixture was allowed to warm up to 0° C. and stirred for 10 minutes before letting it warm to room temperature. The layers were separated, and the aqueous layer was extracted with EtOAc (3×20 mL). The combined organic layers were washed with brine, dried over anhydrous sodium sulfate and concentrated under reduced pressure. The crude product was purified by silica gel column chromatography (0-20% EtOAc in Hexanes) to afford methyl 3-[[4-chloro-6-(2-isopentyl-6-methyl-phenyl)-5-methyl-pyrimidin-2-yl]sulfamoyl]benzoate (1.067 g, 85%) as a colorless oil. ESI-MS m/z calc. 501.1489, found 502.1 (M+1)⁺; Retention time: 3.99 minutes; LC method E.

### Step 10: 3-[[4-Chloro-6-(2-isopentyl-6-methyl-phenyl)-5-methyl-pyrimidin-2-yl]sulfamoyl]benzoic acid

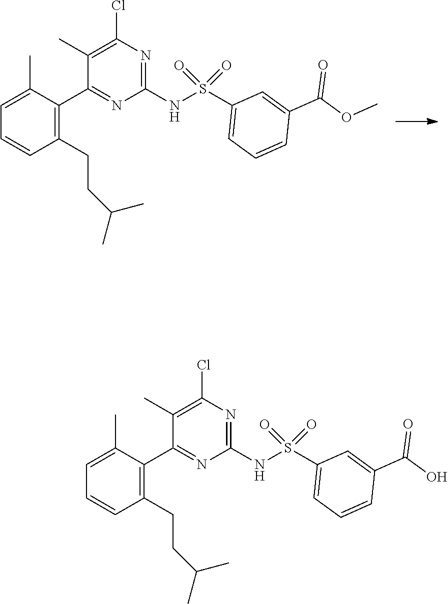 [SMILES: COC(=O)c1cccc(S(=O)(=O)Nc2nc(Cl)c(C)c(-c3c(C)cccc3CCC(C)C)n2)c1 ; Cc1cccc(CCC(C)C)c1-c1nc(NS(=O)(=O)c2cccc(C(=O)O)c2)nc(Cl)c1C]

To a solution of methyl 3-[[4-chloro-6-(2-isopentyl-6-methyl-phenyl)-5-methyl-pyrimidin-2-yl]sulfamoyl]benzoate (1.067 g, 2.0829 mmol) in THE (20 mL) was added NaOH (17 mL of 1 M, 17.000 mmol) and stirred at room temperature for 3 h. After completion, 2M HCl (12 mL) was added to acidify the solution. The two layers were separated and the aqueous layer was extracted with DCM (3×15 mL), the combined organic layers were dried over anhydrous sodium sulfate and concentrated under reduced pressure to afford 3-[[4-chloro-6-(2-isopentyl-6-methyl-phenyl)-5-methyl-pyrimidin-2-yl]sulfamoyl]benzoic acid (1.051 g, 99%) as a white solid. ¹H NMR (500 MHz, DMSO-d6) δ 13.35 (s, 1H), 12.20 (s, 1H), 8.43-8.36 (m, 1H), 8.19-8.01 (m, 2H), 7.68-7.56 (m, 1H), 7.33-7.16 (m, 1H), 7.16-7.03 (m, 2H), 2.07-1.98 (m, 1H), 1.91-1.86 (m, 1H), 1.84 (s, 3H), 1.74 (s, 3H), 1.20-1.13 (m, 1H), 1.11-1.03 (m, 1H), 1.01-0.94 (m, 1H), 0.60-0.42 (m, 6H). ESI-MS m/z calc. 487.13324, found 488.2 (M+1)⁺; Retention time: 2.9 minutes; LC method H.

### Step 11: 3-[[4-[(2R)-2-amino-4,4-dimethyl-pentoxy]-6-(2-isopentyl-6-methyl-phenyl)-5-methyl-pyrimidin-2-yl]sulfamoyl]benzoic acid

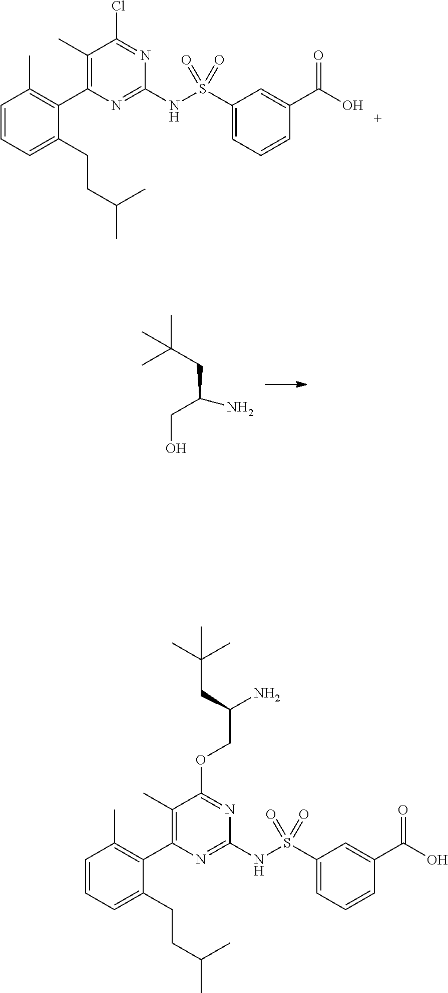

To a vigorously stirring, pre-sonicated suspension of 3-[[4-chloro-6-(2-isopentyl-6-methyl-phenyl)-5-methyl-pyrimidin-2-yl]sulfamoyl]benzoic acid (511 mg, 1.0367 mmol) and (2R)-2-amino-4,4-dimethyl-pentan-1-ol (hydrochloride salt) (527 mg, 3.1430 mmol) in anhydrous THE (10 mL) at 0° C. under nitrogen was portionwise added sodium tert-butoxide (945 mg, 9.8332 mmol). The reaction mixture was allowed to warm up to room temperature and stirred for 4 hours. The reaction mixture was cooled to 0° C., and slowly quenched with 2 M aqueous HCl (9 mL). The reaction mixture was allowed to warm up to room temperature and stirred for 10 minutes. Ethyl acetate (10 mL) was added, and the reaction mixture was vigorously stirred for 10 minutes. The two layers were separated, and the aqueous layer was extracted with ethyl acetate (3×15 mL). Combined organic layers were washed with brine, dried over anhydrous sodium sulfate and concentrated to afford 3-[[4-[(2R)-2-amino-4,4-dimethyl-pentoxy]-6-(2-isopentyl-6-methyl-phenyl)-5-methyl-pyrimidin-2-yl]sulfamoyl]benzoic acid (hydrochloride salt) (547 mg, 77%) as an off white solid. ¹H NMR (500 MHz, DMSO-d6) δ 8.51-8.39 (m, 1H), 8.18-7.99 (m, 2H), 7.72-7.54 (m, 1H), 7.38-7.24 (m, 1H), 7.21-7.01 (m, 2H), 4.46-4.30 (m, 1H), 4.19 (d, J=5.2 Hz, 1H), 4.10-3.93 (m, 1H), 3.67-3.50 (m, 1H), 2.30-2.13 (m, 1H), 2.13-2.00 (m, 1H), 1.99-1.84 (m, 3H), 1.73-1.65 (m, 3H), 1.64-1.45 (m, 2H), 1.37-1.03 (m, 2H), 0.93 (s, 4H), 0.90 (s, 5H), 0.69-0.49 (m, 6H). ESI-MS m/z calc. 582.2876, found 583.3 (M+1)⁺; Retention time: 2.14 minutes; LC method H.

### Example 36: Preparation of 3-[[4-[(2R)-2-amino-4,4-dimethyl-pentoxy]-6-(2-isopropoxy-6-methyl-phenyl)-5-methyl-pyrimidin-2-yl]sulfamoyl]benzoic acid

### Step 1: 2-Bromo-1-isopropoxy-3-methyl-benzene

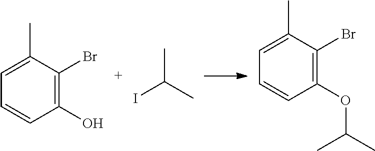

To a solution of 2-bromo-3-methyl-phenol (85.1 g, 455.00 mmol) in anhydrous DMF (700 mL) was added potassium carbonate (125.6 g, 908.79 mmol) and 2-iodopropane (155 g, 911.81 mmol). The mixture was then heated to 100° C. and stirred for 18 hours. The mixture then allowed to cool to rt then quenched with water (250 mL) and EtOAc (250 mL). The aqueous layer was extracted with EtOAc (2×200 mL). The organic layers were combined, washed with brine, dried with anhydrous sodium sulfate, filtered, and concentrated under reduced pressure. The crude was then purified via silica gel column chromatography (load SiO₂) (eluting 0-20% EtOAc in Hexanes) to yield 2-bromo-1-isopropoxy-3-methyl-benzene (85.1 g, 78%) as a yellow oil. ¹H NMR (500 MHz, Chloroform-d) δ 7.12 (t, J=7.9 Hz, 1H), 6.88-6.82 (m, 1H), 6.76 (dd, J=8.3, 1.4 Hz, 1H), 4.54 (h, J=6.0, Hz, 1H), 2.42 (s, 3H), 1.39 (d, J=6.1 Hz, 6H).

### Step 2: (2-Isopropoxy-6-methyl-phenyl)boronic acid

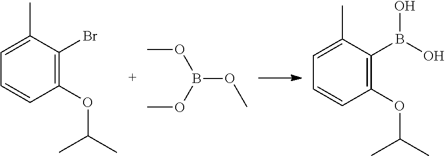

To a solution of 2-bromo-1-isopropoxy-3-methyl-benzene (21 g, 91.657 mmol) in anhydrous THE (850 mL) was added t-BuLi in pentane (120 mL of 1.6 M, 192.00 mmol) at −78° C. The solution was stirred at this temperature for 60 minutes before adding trimethyl borate (39.144 g, 42 mL, 376.70 mmol) dropwise at the same temperature. The solution was stirred at −78° C. for 120 minutes. The solution was then allowed to warm to rt before adding 1 M HCl (300 mL) and stirred overnight. The aqueous layer was then extracted with EtOAc (2×200 mL). The organic layers were combined, washed with brine, dried with anhydrous sodium sulfate, filtered, and concentrated under reduced pressure. The crude was then purified via silica gel column chromatography (load SiO₂)(eluting 0-50% Diethyl ether in Hexanes) to yield (2-isopropoxy-6-methyl-phenyl)boronic acid (13.9 g, 77%) as a white solid. ¹H NMR (500 MHz, DMSO-d6) δ 7.86 (s, 2H), 7.08 (t, J=7.8, 7.8 Hz, 1H), 6.82-6.54 (m, 2H), 4.46 (p, J=6.0 Hz, 1H), 2.20 (s, 3H), 1.22 (d, J=6.0 Hz, 6H). ESI-MS m/z calc. 194.11142, Retention time: 1.69 minutes; LC method H.

### Step 3: tert-Butyl N-tert-butoxycarbonyl-N-[4-chloro-6-(2-isopropoxy-6-methyl-phenyl)-5-methyl-pyrimidin-2-yl]carbamate

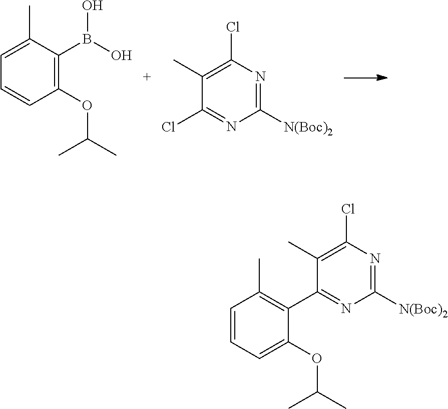

A solution of (2-isopropoxy-6-methyl-phenyl)boronic acid (16.1 g, 78.826 mmol), tert-butyl N-tert-butoxycarbonyl-N-(4,6-dichloro-5-methyl-pyrimidin-2-yl)carbamate (38.5 g, 94.659 mmol), and Cs₂CO₃ (51.5 g, 158.06 mmol) in a solvent mixture of dimethoxyethane (440 mL) and water (150 mL) was degassed with nitrogen for 20 minutes. Pd(dppf)Cl₂ (5.8 g, 7.9267 mmol) was added, and the mixture was purged with nitrogen for another 20 minutes before being sealed and heated to 80° C. for 4 hours. The reaction was quenched with water (250 mL) and EtOAc (250 mL). The aqueous layer was extracted with EtOAc (2×150 mL). The organic layers were combined, washed with brine, dried with anhydrous sodium sulfate, filtered, and concentrated under reduced pressure. The crude was then purified via silica gel column chromatography (load SiO₂) (eluting 0-20% EtOAc in Hexanes) to yield tert-butyl N-tert-butoxycarbonyl-N-[4-chloro-6-(2-isopropoxy-6-methyl-phenyl)-5-methyl-pyrimidin-2-yl]carbamate (19 g, 47%) as a white solid. ESI-MS m/z calc. 491.2187, found 492.2 (M+1)⁺; Retention time: 4.32 minutes; LC method E.

### Step 4: 4-Chloro-6-(2-isopropoxy-6-methyl-phenyl)-5-methyl-pyrimidin-2-amine

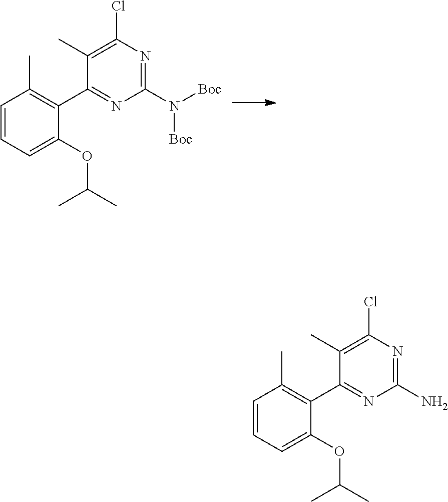 [SMILES: Cc1cccc(OC(C)C)c1-c1nc(N(C(=O)OC(C)(C)C)C(=O)OC(C)(C)C)nc(Cl)c1C ; Cc1cccc(OC(C)C)c1-c1nc(N)nc(Cl)c1C]

To a RBF charged with tert-butyl N-tert-butoxycarbonyl-N-[4-chloro-6-(2-isopropoxy-6-methyl-phenyl)-5-methyl-pyrimidin-2-yl]carbamate (11.8 g, 19.187 mmol) was added HCl in Dioxane (65 mL of 4 M, 260.00 mmol). The solution was stirred over the weekend. The solution was concentrated under reduced pressure to yield a solid that was then triturated with hexanes (70 mL) for 1 hour. The solid was then filtered and neutralized with saturated aqueous sodium bicarbonate (70 mL). The mixture was then partitioned with EtOAc (70 mL). The aqueous layer was then extracted with EtOAc (2×50 mL). The organic layers were then combined, washed with brine, dried with anhydrous sodium sulfate, filtered, and concentrated under reduced pressure to yield 4-chloro-6-(2-isopropoxy-6-methyl-phenyl)-5-methyl-pyrimidin-2-amine (5.9 g, 102%) as an off white solid. ¹H NMR (500 MHz, DMSO-d6) δ 7.28-7.18 (m, 1H), 6.91 (d, J=8.3 Hz, 1H), 6.84 (d, J=7.6, 0.9, 0.9 Hz, 1H), 6.78 (s, 2H), 4.50 (p, J=6.0 Hz, 1H), 1.95 (s, 3H), 1.80 (d, J=0.8 Hz, 3H), 1.13 (d, J=6.0 Hz, 3H), 1.09 (d, J=6.0 Hz, 3H). ESI-MS m/z calc. 291.11383, found 292.0 (M+1)⁺; Retention time: 2.3 minutes; LC method H.

### Step 5: Methyl 3-[[4-chloro-6-(2-isopropoxy-6-methyl-phenyl)-5-methyl-pyrimidin-2-yl]sulfamoyl]benzoate

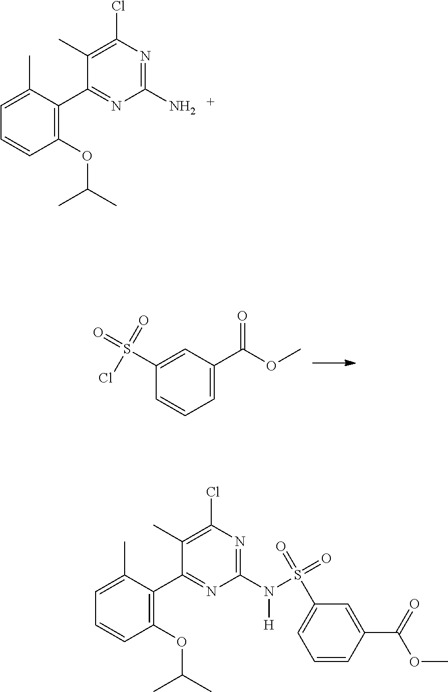 [SMILES: Cc1cccc(OC(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3OC(C)C)n2)c1]

To a solution of 4-chloro-6-(2-isopropoxy-6-methyl-phenyl)-5-methyl-pyrimidin-2-amine (2.28 g, 7.4235 mmol) and methyl 3-chlorosulfonylbenzoate (5.2 g, 22.160 mmol) in THE (45.600 mL) was added LiHMDS in THE (32 mL of 1 M, 32.000 mmol) dropwise over twenty minutes at −78° C. The reaction mixture was stirred for 2 h and then quenched with 1M hydrochloric acid (100 mL). The layers were separated, and the aqueous layer was extracted with ethyl acetate (2×250 mL). The combined organic layer was washed with brine, dried over sodium sulfate, filtered and concentrated in vacuo. The resulting yellow oil was purified by ISCO Companion (SiO₂, 120 g, loaded in EtOAc, eluted with 10% ethyl acetate in hexanes). The desired product fractions were combined and concentrated in vacuo to give methyl 3-[[4-chloro-6-(2-isopropoxy-6-methyl-phenyl)-5-methyl-pyrimidin-2-yl]sulfamoyl]benzoate (3.5 g, 91%) as a yellow sticky solid. ESI-MS m/z calc. 489.11252, found 490.5 (M+1)⁺; Retention time: 6.18 minutes; LC method D.

### Step 6: 3-[[4-Chloro-6-(2-isopropoxy-6-methyl-phenyl)-5-methyl-pyrimidin-2-yl]sulfamoyl]benzoic acid

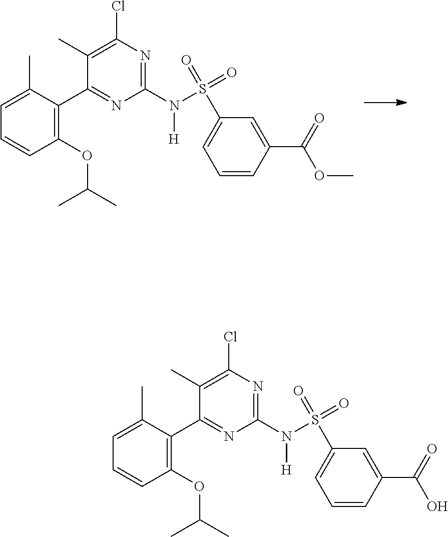 [SMILES: COC(=O)c1cccc(S(=O)(=O)Nc2nc(Cl)c(C)c(-c3c(C)cccc3OC(C)C)n2)c1 ; Cc1cccc(OC(C)C)c1-c1nc(NS(=O)(=O)c2cccc(C(=O)O)c2)nc(Cl)c1C]

To a solution of methyl 3-[[4-chloro-6-(2-isopropoxy-6-methyl-phenyl)-5-methyl-pyrimidin-2-yl]sulfamoyl]benzoate (3.5 g, 6.7861 mmol) in THE (35 mL) was added NaOH (30 mL of 1 M, 30.000 mmol) and the reaction was stirred at rt for 2 h. The reaction mixture was diluted with water (25 mL) and extracted with ethyl acetate (50 mL). The aqueous layer was acidified to pH=1 using 1 N hydrochloric acid and then extracted with ethyl acetate (2×50 mL). The organic layer was washed with brine, dried over sodium sulfate, filtered and concentrated in vacuo to give 3-[[4-chloro-6-(2-isopropoxy-6-methyl-phenyl)-5-methyl-pyrimidin-2-yl]sulfamoyl]benzoic acid (3.2 g, 94%) as a white solid. ESI-MS m/z calc. 475.09686, found 476.3 (M+1)⁺; Retention time: 5.43 minutes; LC method D.

### Step 7: 3-[[4-[(2R)-2-Amino-4,4-dimethyl-pentoxy]-6-(2-isopropoxy-6-methyl-phenyl)-5-methyl-pyrimidin-2-yl]sulfamoyl]benzoic acid

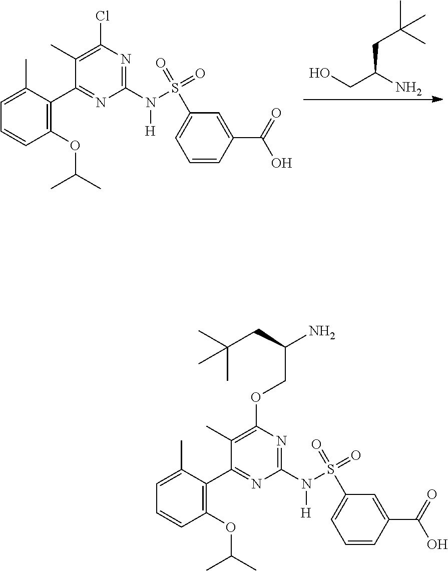

A solution of 3-[[4-chloro-6-(2-isopropoxy-6-methyl-phenyl)-5-methyl-pyrimidin-2-yl]sulfamoyl]benzoic acid (3.2 g, 6.3873 mmol)3-[[4-chloro-6-(2-isopropoxy-6-methyl-phenyl)-5-methyl-pyrimidin-2-yl]sulfamoyl]benzoic acid (3.2 g, 6.3873 mmol), (2R)-2-amino-4,4-dimethyl-pentan-1-ol (2.0 g, 15.242 mmol)(2R)-2-amino-4,4-dimethyl-pentan-1-ol (2.0 g, 15.242 mmol) and sodium t-butoxide (3.5 g, 36.419 mmol) in THE (64.000 mL) was stirred at rt for 2 h. The reaction mixture was quenched with 1 M HCl (40 mL) and extracted with DCM (6×50 mL). The combined organic layer was washed with brine, dried over sodium sulfate, filtered and concentrated in vacuo to give 3-[[4-[(2R)-2-amino-4,4-dimethyl-pentoxy]-6-(2-isopropoxy-6-methyl-phenyl)-5-methyl-pyrimidin-2-yl]sulfamoyl]benzoic acid (hydrochloride salt) (3.6 g, 83%) as a pale yellow solid. ¹H NMR (500 MHz, DMSO-d6) δ 12.85 (s, 1H), 8.44-8.41 (m, 1H), 8.15 (s, 3H), 8.11-8.06 (m, 2H), 7.69-7.64 (m, 1H), 7.34-7.25 (m, 1H), 6.98-6.92 (m, 1H), 6.90-6.84 (m, 1H), 4.58-4.45 (m, 1H), 4.29-4.18 (m, 1H), 4.09-4.03 (m, 1H), 3.61-3.53 (m, 1H), 1.99-1.88 (m, 5H), 1.69 (s, 3H), 1.12-1.06 (m, 6H), 0.92 (s, 4H), 0.89 (s, 5H). ESI-MS m/z calc. 570.2512, found 571.3 (M+1)⁺; Retention time: 1.89 minutes; LC method H.

### Example 37: Preparation of 6-isopropylfuro[2,3-b]pyrazine-2-carbaldehyde

### Step 1: 2-[(4-Methoxyphenyl)methylamino]acetonitrile

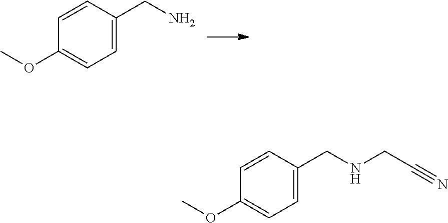

To a solution of (4-methoxyphenyl)methanamine (1.0500 g, 1 mL, 7.6542 mmol) in THE (15 mL) at 0° C., triethylamine (871.20 mg, 1.2 mL, 8.6095 mmol) was added. bromoacetonitrile (1.0320 g, 0.6 mL, 8.6037 mmol) was slowly added and stirred for 10 minutes. The reaction mixture was slowly warmed to room temperature and stirred for 16 h. After 16 h, the reaction mixture was concentrated in vacuo, diluted with ethyl acetate (10 mL) and water (10 mL). The aqueous layer was extracted with ethyl acetate (2×20 mL). The combined organic layers were washed with brine (20 mL), dried with sodium sulfate, filtered and concentrated in vacuo. The residue was purified by silica gel chromatography in 60% ethyl acetate in hexanes to furnish a clear oil. To a suspension of clear oil in diethyl ether, HCl/Dioxane (2.7 mL of 4 M, 10.800 mmol) was added and the resulting mixture was filtered and the filter cake was washed with diethyl ether to afford 2-[(4-methoxyphenyl)methyl amino]acetonitrile (hydrochloride salt) (1.45 g, 88%) as a pale yellow solid. ¹H NMR (400 MHz, DMSO) δ 10.51 (s, 1H), 7.48 (d, J=6.5 Hz, 2H), 6.97 (d, J=6.5 Hz, 2H), 4.24 (s, 2H), 4.12 (s, 2H), 3.76 (s, 3H); ESI-MS m/z calc. 176.09496, found 177.0 (M+1)⁺; Retention time: 1.23 minutes; LC method E.

### Step 2: 3,5-Dichloro-1-[(4-methoxyphenyl)methyl]pyrazin-2-one

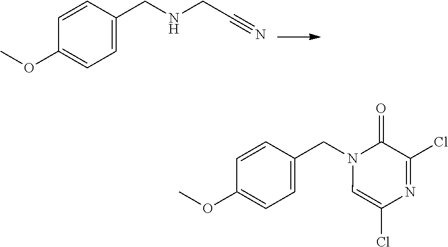 [SMILES: COc1ccc(CNCC#N)cc1 ; COc1ccc(Cn2cc(Cl)nc(Cl)c2=O)cc1]

To a solution of 2-[(4-methoxyphenyl)methylamino]acetonitrile (hydrochloride salt) (1.45 g, 6.8179 mmol) in chlorobenzene (12 mL) at room temperature, oxalyl chloride (2.6190 g, 1.8 mL, 20.634 mmol) was added and the reaction was stirred for 30 minutes. Triethylamine (hydrochloride salt) (4.6 g, 6.3361 mL, 33.418 mmol) was added and the reaction was stirred for 16 h. The reaction mixture is diluted with DCM (100 mL) and washed with water (2×50 mL). The organic layer was washed with brine (50 mL), dried with sodium sulfate, filtered and concentrated in vacuo. The residue was purified by silica gel chromatography in 70% ethyl acetate in hexanes to furnish to give 3,5-dichloro-1-[(4-methoxyphenyl)methyl]pyrazin-2-one (1.28 g, 63%) as a pale yellow oil. ¹H NMR (500 MHz, DMSO) δ 8.12 (s, 1H), 7.21 (d, 2H), 6.83 (d, 2H), 4.92 (s, 2H), 3.63 (s, 3H). ESI-MS m/z calc. 284.01193, found 285.2 (M+1)⁺; Retention time: 2.69 minutes; LC method E.

### Step 3: 5-Chloro-1-[(4-methoxyphenyl)methyl]-3-(3-methylbut-1-ynyl)pyrazin-2-one

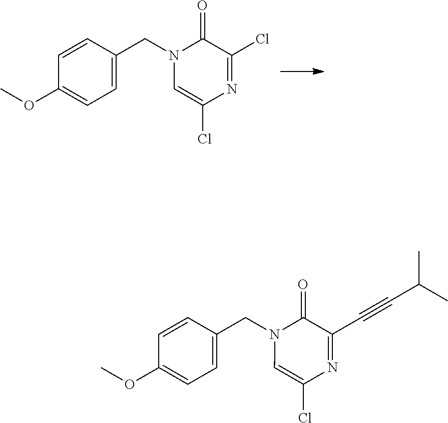 [SMILES: COc1ccc(Cn2cc(Cl)nc(Cl)c2=O)cc1 ; COc1ccc(Cn2cc(Cl)nc(C#CC(C)C)c2=O)cc1]

To a microwave vial 3,5-dichloro-1-[(4-methoxyphenyl)methyl]pyrazin-2-one (1.08 g, 3.7878 mmol), bis(triphenylphosphine)palladium(II) dichloride (28 mg, 0.0399 mmol), CuI (22 mg, 0.1155 mmol) in DMF (6 mL) and Et₃N (6 mL), isopropylacetylene (396.00 mg, 0.6 mL, 5.8135 mmol) was added and sealed. The microwave vial was irradiated at 80° C. for 10 min. The reaction mixture was allowed to cool, was diluted with DCM (50 mL) and washed with water (2×50 mL). The organic layer was washed with brine, dried with sodium sulfate, filtered and concentrated in vacuo. The residue was purified by silica gel chromatography in 50% ethyl acetate in hexanes to furnish to give 5-chloro-1-[(4-methoxyphenyl)methyl]-3-(3-methylbut-1-ynyl)pyrazin-2-one (1.02 g, 83%) as a pale yellow solid. ¹H NMR (500 MHz, CDCl₃) δ 7.28 (d, J=8.6 Hz, 2H), 7.10 (s, 1H), 6.90 (d, J=6.6 Hz, 2H), 4.99 (s, 2H), 3.81 (s, 3H), 2.89 (dt, J=13.7, 6.9 Hz, 1H), 1.30 (d, 6H). ESI-MS m/z calc. 316.09787, found 317.1 (M+1)⁺; Retention time: 3.12 minutes; LC method E.

### Step 4: 2-Chloro-6-isopropyl-furo[2,3-b]pyrazine

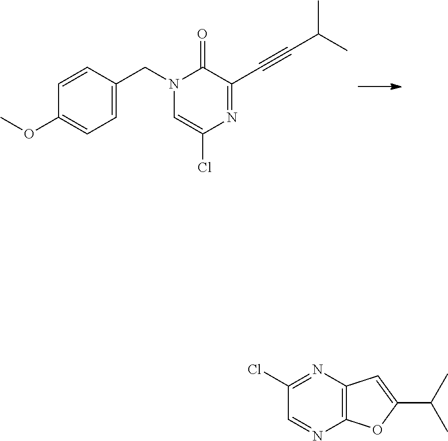 [SMILES: COc1ccc(Cn2cc(Cl)nc(C#CC(C)C)c2=O)cc1 ; CC(C)c1cc2nc(Cl)cnc2o1]

To a solution of 5-chloro-1-[(4-methoxyphenyl)methyl]-3-(3-methylbut-1-ynyl)pyrazin-2-one (970 mg, 3.0620 mmol) in DCM (20 mL), trifluoromethanesulfonate (20 mg, 0.0778 mmol) and TFA (2.9600 g, 2 mL, 25.960 mmol) were added and the reaction was stirred at room temperature for 30 minutes. The residue was concentrated in vacuo and purified by silica gel chromatography in 30% ethyl acetate in hexanes to furnish 2-chloro-6-isopropyl-furo[2,3-b]pyrazine (510 mg, 84%) as a pale yellow solid. ¹H NMR (500 MHz, CDCl₃) δ 8.15 (s, 1H), 6.56 (s, 1H), 3.17 (dt, J=13.8, 6.9 Hz, 1H), 1.40 (d, J=6.9 Hz, 6H). ESI-MS m/z calc. 196.04034, found 197.1 (M+1)⁺; Retention time: 3.14 minutes; LC method E.

### Step 5: Methyl 6-isopropylfuro[2,3-b]pyrazine-2-carboxylate

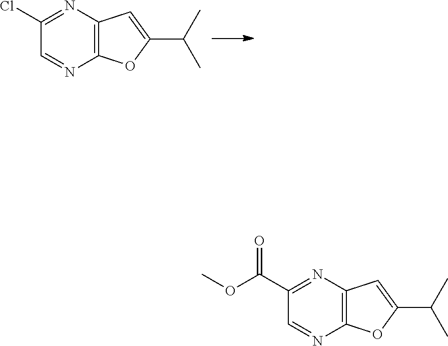 [SMILES: CC(C)c1cc2nc(Cl)cnc2o1 ; COC(=O)c1cnc2oc(C(C)C)cc2n1]

A mixture of 2-chloro-6-isopropyl-furo[2,3-b]pyrazine (260 mg, 1.3223 mmol) and Pd(dppf)₂Cl₂CH₂Cl₂ (105 mg, 0.1286 mmol) and Et₃N (580.80 mg, 0.8 mL, 5.7397 mmol) in MeOH (15 mL) in a steel bomb equipped with mechanical stirrer was purged with carbon monoxide three times. The reaction mixture was heated to 100° C. with 120 psi of carbon monoxide and stirred for 14 h. The reaction mixture was allowed to cool to rt over 1 h. The reaction mixture was filtered through a pad of celite, washed with ethyl acetate, and the filtrate was concentrated in vacuo. The residue was purified by silica gel chromatography in 50% ethyl acetate in hexanes to give methyl 6-isopropylfuro[2,3-b]pyrazine-2-carboxylate (236 mg, 80%) as a pale yellow solid; ¹H NMR (400 MHz, CDCl₃) δ 9.02 (s, 1H), 6.72 (d, J=1.0 Hz, 1H), 4.07 (s, 3H), 3.20 (dq, J=13.7, 6.8 Hz, 1H), 1.43 (d, J=6.9 Hz, 6H); ESI-MS m/z calc. 220.0848, found 221.4 (M+1)⁺; Retention time: 2.54 minutes; LC method E.

### Step 6: 6-Isopropylfuro[2,3-b]pyrazine-2-carbaldehyde

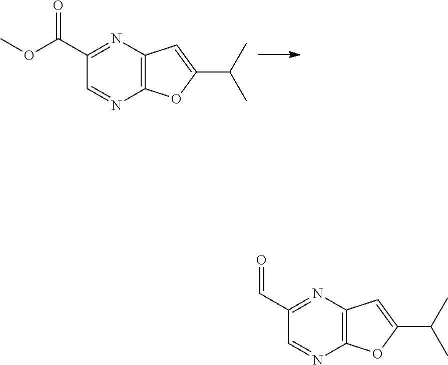 [SMILES: COC(=O)c1cnc2oc(C(C)C)cc2n1 ; CC(C)c1cc2nc(C=O)cnc2o1]

To a stirred solution of methyl 6-isopropylfuro[2,3-b]pyrazine-2-carboxylate (400 mg, 1.8163 mmol) in DCM (8 mL) was added DIBAL in DCM (3.2 mL of 1 M, 3.2000 mmol) at −78° C. over 2 minutes. The reaction mixture was stirred for 2 h, quenched with MeOH (1 mL)/water (1 mL) and concentrated in vacuo. DCM (10 mL) was added and the reaction was filtered. The cake was washed with DCM. The organic layer from the filtrate was separated, washed with brine, dried over sodium sulfate, filtered, and concentrated in vacuo. The resulting yellow oil was purified by silica gel chromatography (Silica, 12 g, loaded with DCM, eluted with 70% ethyl acetate in hexanes). The desired product fractions were combined and concentrated in vacuo to give 6-isopropylfuro[2,3-b]pyrazine-2-carbaldehyde (254 mg, 73%) as a pale yellow solid. ¹H NMR (500 MHz, DMSO) δ 10.11 (s, 1H), 8.81 (s, 1H), 7.11 (s 1H), 3.22 (dt, J=13.7, 6.9 Hz, 1H), 1.35 (d, J=6.9 Hz, 6H). ESI-MS m/z calc. 190.07423, found 191.2 (M+1)⁺; Retention time: 1.92 minutes; LC method E.

### Example 38: Preparation of 6-isopropyl-5-methyl-pyrrolo[2,3-b]pyrazine-3-carbaldehyde

### Step 1: 6-Chloro-3-(3-methylbut-1-ynyl)pyrazin-2-amine

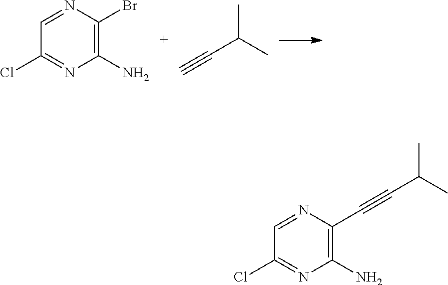

To a stirred solution of 3-bromo-6-chloro-pyrazin-2-amine (5 g, 23.987 mmol) in triethylamine (50 mL) was added 3-methylbut-1-yne (2.6640 g, 4 mL, 39.109 mmol). Nitrogen was bubbled into the mixture for 10 minutes and then bis(triphenylphosphine)palladium(II)chloride (1.7 g, 2.4220 mmol) followed by CuI (460 mg, 2.4153 mmol) was added. Nitrogen was bubbled into the mixture for 5 minutes. The mixture was stirred at room temperature for 90 minutes. The mixture was diluted by EtOAc (100 mL), was filtered on a pad of Celite and the pad was rinsed with EtOAc (100 mL). Water (100 mL) was then added, and the organic phase was washed with water (3×250 mL) and brine (1×20 mL), dried over sodium sulfate, filtered and concentrated in vacuo. The crude mixture was purified by flash-chromatography on a 120 g cartridge, eluting with a gradient of 0 to 50% of EtOAc in heptanes to afford after evaporation 6-chloro-3-(3-methylbut-1-ynyl)pyrazin-2-amine (4.5 g, 96%) as a beige solid. ¹H NMR (400 MHz, CDCl₃) δ 7.86 (s, 1H), 5.14 (br. s, 2H), 2.88 (spt, J=6.9 Hz, 1H), 1.32 (d, J=6.8 Hz, 6H). ESI-MS m/z calc. 195.05632, found 196.2 (M+1)⁺; Retention time: 1.7 minutes; LC method I.

### Step 2: 3-Chloro-6-isopropyl-5H-pyrrolo[2,3-b]pyrazine

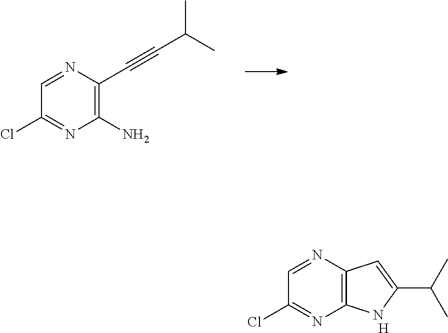

To a solution of 6-chloro-3-(3-methylbut-1-ynyl)pyrazin-2-amine (4.5 g, 22.977 mmol) in tert-butanol (50 mL) was added potassium tert-butoxide (11.5 g, 102.48 mmol). The mixture was stirred at 80° C. for 18 hours and the mixture was then cooled down to room temperature. The crude mixture was evaporated in vacuo to dryness. Water (100 mL) was then added, and the mixture was stirred at room temperature for 30 minutes. The suspension was then filtered, the solid was washed with water (100 mL) and then the solid was co-evaporated with MeCN (2×50 mL) to afford 3-chloro-6-isopropyl-5H-pyrrolo[2,3-b]pyrazine (4.45 g, 99%) as a beige solid. ¹H NMR (400 MHz, DMSO-d6) δ 12.22 (br. s, 1H), 8.34 (s, 1H), 6.41 (s, 1H), 3.10 (spt, J=6.8 Hz, 1H), 1.32 (d, J=7.1 Hz, 6H). ESI-MS m/z calc. 195.05632, found 196.2 (M+1)⁺; Retention time: 1.64 minutes; LC method I.

### Step 3: 3-Chloro-6-isopropyl-5-methyl-pyrrolo[2,3-b]pyrazine

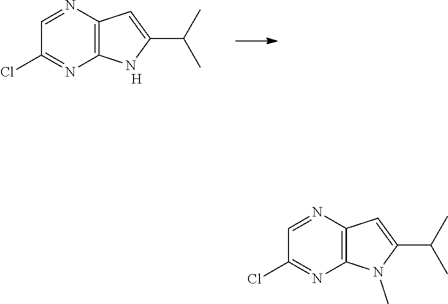

To a solution of 3-chloro-6-isopropyl-5H-pyrrolo[2,3-b]pyrazine (4.4 g, 22.467 mmol) in DMF (60 mL) at 0° C. was added NaH (in mineral dispersion) (2.5 g, 60% w/w, 62.506 mmol). The mixture was stirred at 0° C. for 5 minutes and then dimethyl sulfate (5.9850 g, 4.5 mL, 47.450 mmol) was added. The mixture was stirred at 0° C. for 5 minutes and then at room temperature for 3 hours. The mixture was then cooled down to 0° C. and water (100 mL) was added. EtOAc (250 mL) was added, and the mixture was extracted with EtOAc (3×250 mL). The combined organic layers were washed with water (3×250 mL) and brine (3×250 mL), dried over sodium sulfate, filtered, and concentrated in vacuo. The crude mixture was purified by flash-chromatography on a silica gel cartridge (120 g Gold), eluting with a gradient of 0 to 50% of EtOAc in heptane to afford after evaporation 3-chloro-6-isopropyl-5-methyl-pyrrolo[2,3-b]pyrazine (4 g, 85%) as a yellow oil. ¹H NMR (400 MHz, CDCl₃) δ 8.34 (s, 1H), 6.45 (s, 1H), 3.81 (s, 3H), 3.15 (spt, J=6.8 Hz, 1H), 1.39 (d, J=6.8 Hz, 6H). ESI-MS m/z calc. 209.07198, found 210.2 (M+1)⁺; Retention time: 1.81 minutes; LC method I.

### Step 4: Methyl 6-isopropyl-5-methyl-pyrrolo[2,3-b]pyrazine-3-carboxylate

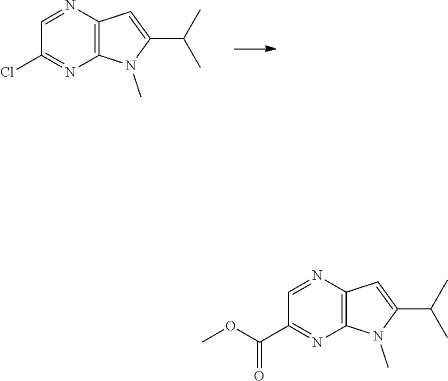 [SMILES: CC(C)c1cc2ncc(Cl)nc2n1C ; COC(=O)c1cnc2cc(C(C)C)n(C)c2n1]

To a solution of 3-chloro-6-isopropyl-5-methyl-pyrrolo[2,3-b]pyrazine (4 g, 19.058 mmol) in MeOH (20 mL) was added triethylamine (3.9930 g, 5.5 mL, 39.460 mmol). Nitrogen was bubbled into the mixture for 15 minutes and then 1,1′-Bis(diphenylphosphino)ferrocene palladium (II) chloride, complex with dichloromethane (775 mg, 0.9490 mmol) was added. Nitrogen was bubbled into the mixture for 5 minutes. The mixture was then stirred at 100° C. under 50 psi carbon monoxide pressure for 18 hours. The mixture was then cooled down to room temperature, was filtered on a Celite pad and was concentrated in vacuo. Water (100 mL) and EtOAc (100 mL) were then added, and the mixture was extracted with EtOAc (3×100 mL). The combined organic layers were washed with brine (100 mL), dried over sodium sulfate, filtered, and concentrated in vacuo. The crude mixture was purified by flash-chromatography on a 120 g cartridge, eluting with a gradient of 0 to 50% of EtOAc in heptane to afford after evaporation methyl 6-isopropyl-5-methyl-pyrrolo[2,3-b]pyrazine-3-carboxylate (4.11 g, 92%) as a beige solid. ¹H NMR (400 MHz, CDCl₃) δ 9.17 (s, 1H), 6.55 (s, 1H), 4.04 (s, 3H), 3.92 (s, 3H), 3.21 (spt, J=6.8 Hz, 1H), 1.42 (d, J=6.6 Hz, 6H). ESI-MS m/z calc. 233.11642, found 234.2 (M+1)⁺; Retention time: 1.59 minutes; LC method I.

### Step 5: (6-Isopropyl-5-methyl-pyrrolo[2,3-b]pyrazin-3-yl)methanol

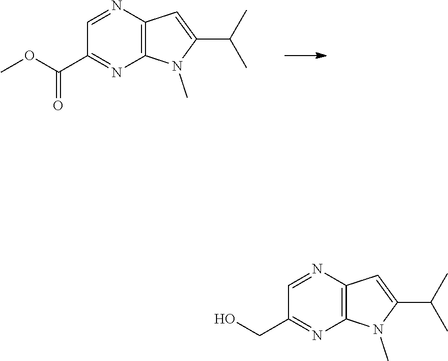 [SMILES: COC(=O)c1cnc2cc(C(C)C)n(C)c2n1 ; CC(C)c1cc2ncc(CO)nc2n1C]

To a solution of LAH (200 mg, 5.2695 mmol) in dry THE (10 mL) at 0° C. was added methyl 6-isopropyl-5-methyl-pyrrolo[2,3-b]pyrazine-3-carboxylate (1 g, 4.2827 mmol) in dry THE (10 mL). The mixture was stirred at 0° C. for 30 minutes. Water (0.2 mL) was added followed by an aqueous solution of NaOH (15%, 0.2 mL) and then by water (0.6 mL). The mixture was stirred at room temperature for 30 minutes and then magnesium sulfate (500 mg) was added. The mixture was filtered on Celite and the filter cake was washed with EtOAc (50 mL). The filtrate was then concentrated in vacuo to afford (6-isopropyl-5-methyl-pyrrolo[2,3-b]pyrazin-3-yl)methanol (750 mg, 81%) as a yellow solid.

### Step 6: 6-Isopropyl-5-methyl-pyrrolo[2,3-b]pyrazine-3-carbaldehyde

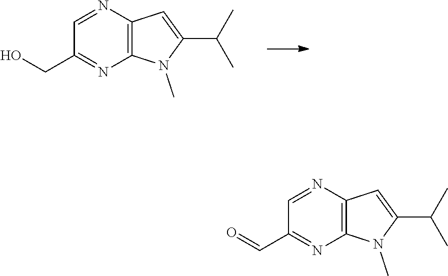

To a 0° C. solution of (6-isopropyl-5-methyl-pyrrolo[2,3-b]pyrazin-3-yl)methanol (2.5 g, 10.548 mmol) in DCM (75 mL) was added Dess-Martin periodinane (7.3 g, 17.211 mmol). The reaction was stirred at 0° C. for 5 minutes and then stirred for 2 hours at room temperature. An aqueous saturated solutions of iN NaOH (20 mL) and water (20 mL) were added and the reaction mixture was stirred for 30 minutes. The mixture was then filtered on a silica pad and the pad was rinsed with DCM (250 mL). Water (250 mL) was added to the mixture and the aqueous one was extracted with DCM (3×250 mL). The combined organic layers were dried over sodium sulfate, filtered and concentrated in vacuo. The crude mixture was purified by flash-chromatography on a 40 g silica gel cartridge, eluting with a gradient of 0 to 100% of EtOAc in heptane to afford after evaporation 6-isopropyl-5-methyl-pyrrolo[2,3-b]pyrazine-3-carbaldehyde (2 g, 85%) as a dark orange solid. ¹H NMR (400 MHz, CDCl₃) δ 10.17 (s, 1H), 9.04 (s, 1H), 6.59 (s, 1H), 3.93 (s, 3H), 3.24 (spt, J=6.8 Hz, 1H), 1.44 (d, J=6.8 Hz, 6H). ESI-MS m/z calc. 203.10587, found 204.1 (M+1)⁺; Retention time: 3.44 minutes; LC method J.

### V. Exemplary Synthesis of Compounds

### Example 39: Preparation of Compound II-2

### Step 1: 3-[[4-[(2R)-2-[(6-Chloropyrazin-2-yl)methylamino]-5,5-dimethyl-hexoxy]-6-(2,6-dimethylphenyl)pyrimidin-2-yl]sulfamoyl]benzoic acid

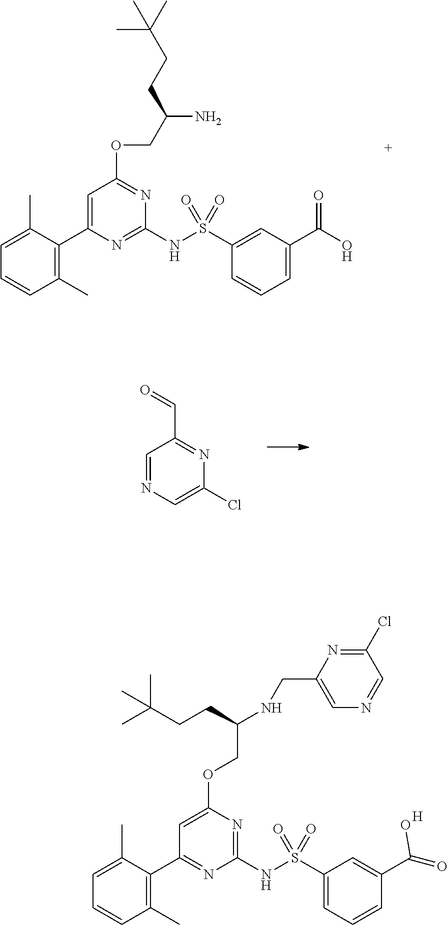

A 100 mL RBF was charged with 3-[[4-[(2R)-2-amino-5,5-dimethyl-hexoxy]-6-(2,6-dimethylphenyl)pyrimidin-2-yl]sulfamoyl]benzoic acid (hydrochloride salt) (2 g, 3.552 mmol), 6-chloropyrazine-2-carbaldehyde (571 mg, 4.006 mmol), anhydrous DCM (15 mL) and acetic acid (330 μL, 5.803 mmol). The mixture was cooled down in an ice bath. DIEA (1.5 mL, 8.612 mmol) was added, followed by sodium triacetoxyborohydride (4 g, 18.87 mmol) and the reaction was vigorously stirred at 0° C. After 1 hour, additional DCM (5 mL) was added to improve stirring. At 3 hours reaction time additional sodium triacetoxyborohydride (1 g, 4.718 mmol) was added. After 4 hours total reaction time, the reaction mixture was quenched into 2M HCl and extracted 4× with ethyl acetate. The combined organics were washed with brine, dried over sodium sulfate, and concentrated to give 3-[[4-[(2R)-2-[(6-chloropyrazin-2-yl)methylamino]-5,5-dimethyl-hexoxy]-6-(2,6-dimethylphenyl)pyrimidin-2-yl]sulfamoyl]benzoic acid (hydrochloride salt) (2.389 g, 62%) ESI-MS m/z calc. 652.22345, found 653.8 (M+1)⁺; Retention time: 0.55 minutes. as a white solid, which was used in the next step without further purification. LC method B.

### Step 2: (11R)-12-[(6-Chloropyrazin-2-yl)methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,4,16-hexaen-13-one

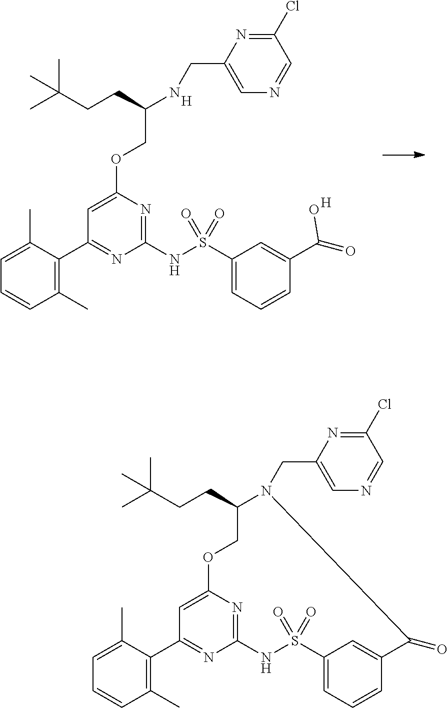

3-[[4-[(2R)-2-[(6-Chloropyrazin-2-yl)methylamino]-5,5-dimethyl-hexoxy]-6-(2,6-dimethylphenyl)pyrimidin-2-yl]sulfamoyl]benzoic acid (hydrochloride salt) (2.39 g, 2.772 mmol) was combined with CDMT (780 mg, 4.443 mmol) in anhydrous DMF (185 mL). The reaction mixture was cooled to 0° C. in an ice bath, and N-methylmorpholine (1.8 mL, 16.37 mmol) was added dropwise over 2 minutes via syringe. The reaction was allowed to slowly warm to room temperature as the ice bath melted for a total of 18 hours stirring. The reaction mixture was concentrated by rotary evaporation (bath temperature 50° C.) to a volume of less than 10 mL. The reaction mixture was partitioned between 1 M HCl in and ethyl acetate. The layers were separated, and the aqueous was extracted 2 additional times with ethyl acetate. The combined organics were washed with water then brine and dried over sodium sulfate. After concentrating the resulting crude material was purified by chromatography on silica gel eluting with a 0-10% DCM in methanol gradient. Overlapping fractions were purified a second time on silica gel under the same gradient. The resulting product fractions were combined to give (11R)-12-[(6-chloropyrazin-2-yl)methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (844.3 mg, 48%) ESI-MS m/z calc. 634.2129, found 635.7 (M+1)⁺; Retention time: 0.78 minutes. LC method B.

### Step 3: (11R)-12-[[6-[(2R,3aS,6aS)-2-Methyl-3,3a,4,5,6,6a-hexahydro-2H-cyclopenta[b]pyrrol-1-yl]pyrazin-2-yl]methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound II-2

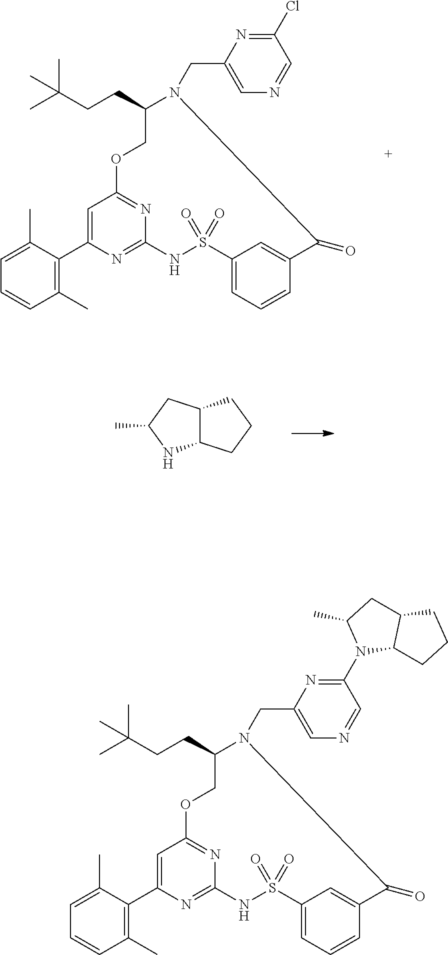

A 4 mL vial was charged with (11R)-12-[(6-chloropyrazin-2-yl)methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (21.8 mg, 0.03432 mmol), (2R,3aS,6aS)-2-methyl-1,2,3,3a,4,5,6,6a-octahydrocyclopenta[b]pyrrole (hydrochloride salt) (27.4 mg, 0.1383 mmol), potassium carbonate (61.0 mg, 0.4414 mmol), DMSO (0.3 mL), and dioxane (0.1 mL). The vial was purged with nitrogen, capped, and stirred at 120° C. for 12 hours. After cooling down to room temperature, the reaction was diluted with DMSO (0.5 mL) and MeOH (0.5 mL), filtered, and purified by reverse phase HPLC (1-99% acetonitrile/5 mM aqueous HCl over 15 min) to give (11R)-12-[[6-[(2R,3aS,6aS)-2-methyl-3,3a,4,5,6,6a-hexahydro-2H-cyclopenta[b]pyrrol-1-yl]pyrazin-2-yl]methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (9.3 mg, 37%). ¹H NMR (400 MHz, CDCl₃) δ 8.76 (s, 1H), 8.03 (d, J=7.9 Hz, 1H), 7.90 (s, 1H), 7.88-7.84 (m, 1H), 7.78 (s, 1H), 7.66 (t, J=7.8 Hz, 1H), 7.21 (t, J=7.6 Hz, 1H), 7.06 (d, J=7.6 Hz, 2H), 6.24 (s, 1H), 5.44-5.39 (m, 1H), 5.13 (d, J=15.0 Hz, 1H), 4.33-4.24 (m, 2H), 4.09-4.00 (m, 3H), 2.86-2.77 (m, 1H), 2.51-2.41 (m, 1H), 2.04 (s, 6H), 1.92-1.83 (m, 2H), 1.83-1.74 (m, 2H), 1.68-1.60 (m, 5H), 1.44 (d, J=6.2 Hz, 3H), 1.20-1.10 (m, 2H), 0.81 (s, 9H). ESI-MS m/z calc. 723.3567, found 724.2 (M+1)⁺; Retention time: 2.05 minutes. LC method A.

### Example 40: Preparation of Compound 11-7

### Step 1: tert-Butyl N-cyclobutylcarbamate

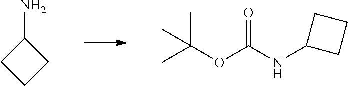

tert-Butoxycarbonyl tert-butyl carbonate (3.38 g, 15.49 mmol) was added to a suspension of cyclobutanamine (1 g, 14.06 mmol) in dichloromethane (10.00 mL). triethylamine (2.35 mL, 16.86 mmol) was added followed by N,N-dimethylpyridin-4-amine (200 mg, 1.637 mmol). The reaction mixture was stirred at room temperature for 72 hours. The reaction mixture was diluted with EtOAc and washed with aqueous 1 M HCl and brine. The organic layer was dried over sodium sulfate filtered and concentrated under reduced pressure to provide tert-butyl N-cyclobutylcarbamate (2.1792 g, 91%).

### Step 2: tert-Butyl N-cyclobutyl-N-(trideuteriomethyl)carbamate

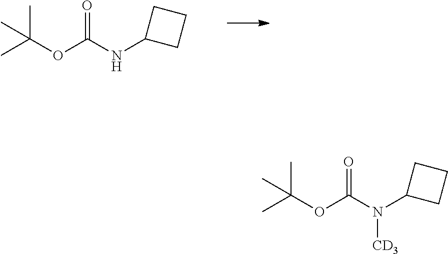 [SMILES: CC(C)(C)OC(=O)NC1CCC1 ; [2H]C([2H])([2H])N(C(=O)OC(C)(C)C)C1CCC1]

A solution of tert-butyl N-cyclobutylcarbamate (200 mg, 1.168 mmol) in THE (2 mL) was cooled to 0° C. before the addition of sodium hydride (70 mg, 1.750 mmol) (60 wt % dispersion in mineral oil). The mixture was stirred for 15 minutes, and trideuterio(iodo)methane (338 mg, 2.332 mmol) was added. Stirring was continued at 0° C. for 30 minutes, and then it allowed to slowly warm to room temperature. After two hours of stirring, trideuterio(iodo)methane (845 mg, 5.829 mmol) was added. The reaction mixture was stirred overnight. To the milky white reaction mixture that was formed, EtOAc (50 mL) was added. It was washed with aqueous HCl (1 M, 1×50 mL) and brine (1×50 mL). The organic layer was dried over sodium sulfate, filtered and concentrated under reduced pressure to provide tert-butyl N-cyclobutyl-N-(trideuteriomethyl)carbamate (212 mg, 96%) as a yellow oil. ESI-MS m/z calc. 188.16042, found 189.2 (M+1)⁺; Retention time: 1.24 minutes. LC method A.

### Step 3: N-(Trideuteriomethyl)cyclobutanamine

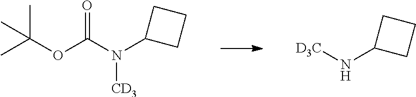 [SMILES: [2H]C([2H])([2H])N(C(=O)OC(C)(C)C)C1CCC1 ; [2H]C([2H])([2H])NC1CCC1]

A solution of HCl in dioxane (3.8 mL of 4 M, 15.20 mmol) was added to tert-butyl N-cyclobutyl-N-(trideuteriomethyl)carbamate (212 mg, 1.126 mmol). The reaction mixture was stirred at room temperature for 30 minutes. Volatiles were removed under reduced pressure to provide N-(trideuteriomethyl)cyclobutanamine (hydrochloride salt) (177 mg, 126%) as a yellow oil. ¹H NMR (400 MHz, Chloroform-d) δ 9.62 (s, 2H), 3.63-3.51 (m, 1H), 2.53-2.41 (m, 2H), 2.36-2.29 (m, 2H), 2.09-1.93 (m, 1H), 1.93-1.75 (m, 1H).

### Step 4: (11R)-12-[[6-[Cyclobutyl(trideuteriomethyl)amino]pyrazin-2-yl]methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one (Compound II-7

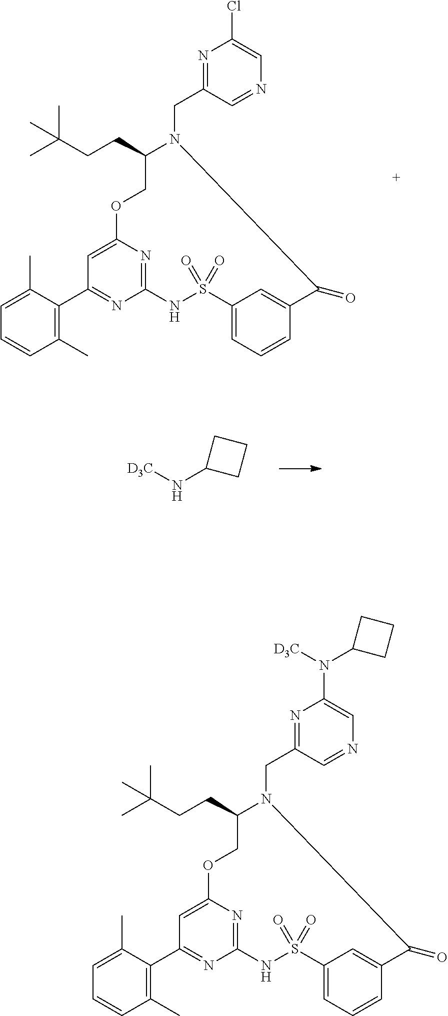 [SMILES: Cc1cccc(C)c1-c1cc2nc(n1)NS(=O)(=O)c1cccc(c1)C(=O)N(Cc1cncc(Cl)n1)[C@H](CCC(C)(C)C)CO2 ; [2H]C([2H])([2H])NC1CCC1 ; [2H]C([2H])([2H])N(c1cncc(CN2C(=O)c3cccc(c3)S(=O)(=O)Nc3nc(cc(-c4c(C)cccc4C)n3)OC[C@H]2CCC(C)(C)C)n1)C1CCC1]

In a 4 mL vial, (11R)-12-[(6-chloropyrazin-2-yl)methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (35 mg, 0.05510 mmol) and N-(trideuteriomethyl)cyclobutanamine (hydrochloride salt) (14 mg, 0.1123 mmol) were combined in DMSO (0.25 mL). Finely ground potassium carbonate (23 mg, 0.1664 mmol) was added. The reaction mixture was stirred at 110° C. over 3 days. After filtration, purification by reverse phase HPLC (10-99% acetonitrile/5 mM aqueous HCl over 15 minutes) gave (11R)-12-[[6-[cyclobutyl(trideuteriomethyl)amino]pyrazin-2-yl]methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one (19.7 mg, 50%) as a yellow solid. ¹H NMR (400 MHz, Chloroform-d) δ 8.74 (s, 1H), 8.06 (d, J=7.9 Hz, 1H), 7.90 (d, J=9.2 Hz, 2H), 7.83 (d, J=7.7 Hz, 1H), 7.66 (t, J=7.7 Hz, 1H), 7.20 (t, J=7.6 Hz, 1H), 7.03 (d, J=7.5 Hz, 2H), 6.16 (s, 1H), 5.37 (d, J=6.6 Hz, 1H), 5.20 (d, J=15.4 Hz, 1H), 4.60-4.49 (m, 1H), 4.07-3.94 (m, 3H), 2.35-2.25 (m, 2H), 2.25-2.13 (m, 3H), 2.00 (s, 6H), 1.78-1.70 (m, 2H), 1.69-1.58 (m, 2H), 1.16 (td, J=12.8, 5.5 Hz, 1H), 0.82 (s, 9H). ESI-MS m/z calc. 686.3442, found 687.5 (M+1)⁺; Retention time: 1.7 minutes. LC method A.

### Example 41: Preparation of Compound III-1

### Step 1: 3-[[4-[(2R)-2-[(7-Bromoimidazo[1,2-a]pyridin-2-yl)methylamino]-4,4-dimethyl-pentoxy]-6-(2,6-dimethylphenyl)pyrimidin-2-yl]sulfamoyl]benzoic acid

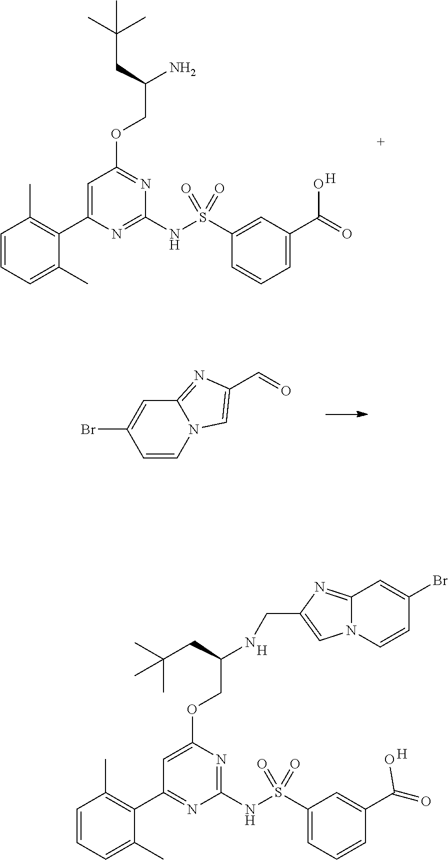

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (350 mg, 0.6374 mmol) and 7-bromoimidazo[1,2-a]pyridine-2-carbaldehyde (145 mg, 0.6443 mmol) in anhydrous dichloromethane (2.5 mL) were added glacial acetic acid (80 μL, 1.407 mmol) and DIPEA (350 μL, 2.009 mmol), in that order, at 0° C. under nitrogen. After 2-3 min, sodium triacetoxyborohydride (470 mg, 2.218 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish 3-[[4-[(2R)-2-[(7-bromoimidazo[1,2-a]pyridin-2-yl)methylamino]-4,4-dimethyl-pentoxy]-6-(2,6-dimethylphenyl)pyrimidin-2-yl]sulfamoyl]benzoic acid (hydrochloride salt) (363 mg, 75%) as a pinkish solid. ESI-MS m/z calc. 720.173, found 723.0 (M+1)⁺; Retention time: 0.54 minutes. LC method B.

### Step 2: (11R)-12-[(7-Bromoimidazo[1,2-a]pyridin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,4,16-hexaen-13-one

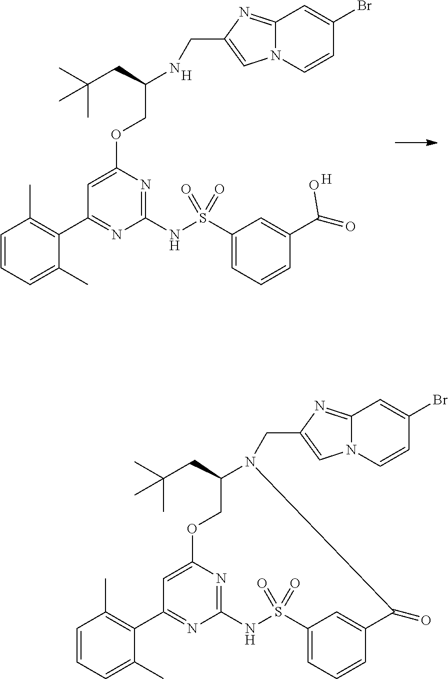

To a stirred solution of 3-[[4-[(2R)-2-[(7-bromoimidazo[1,2-a]pyridin-2-yl)methylamino]-4,4-dimethyl-pentoxy]-6-(2,6-dimethylphenyl)pyrimidin-2-yl]sulfamoyl]benzoic acid (hydrochloride salt) (363 mg, 0.4788 mmol) in anhydrous DMF (17 mL) was added 2-chloro-4,6-dimethoxy-1,3,5-triazine (120 mg, 0.6835 mmol) (CDMT), followed by addition of 4-methylmorpholine (300 μL, 2.729 mmol) at 0-4° C. (ice-water bath) under nitrogen. The yellow reaction was allowed to stir at that temperature for 5 min, then allowed to stir at room temperature for 3 h. The volatiles were removed under reduced pressure and to the residue water (20 mL) was added and stirred for 5 min. The light yellow solid was filtered, washed with water (2×10 mL) and dried in vacuo to furnish (11R)-12-[(7-bromoimidazo[1,2-a]pyridin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (303 mg, 90%) as an off-white solid. ESI-MS m/z calc. 702.1624, found 705.0 (M+3)⁺; Retention time: 1.4 minutes. LC method A.

### Step 3: (11R)-6-(2,6-Dimethylphenyl)-11-(2,2-dimethylpropyl)-12-[(7-isopropenylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one

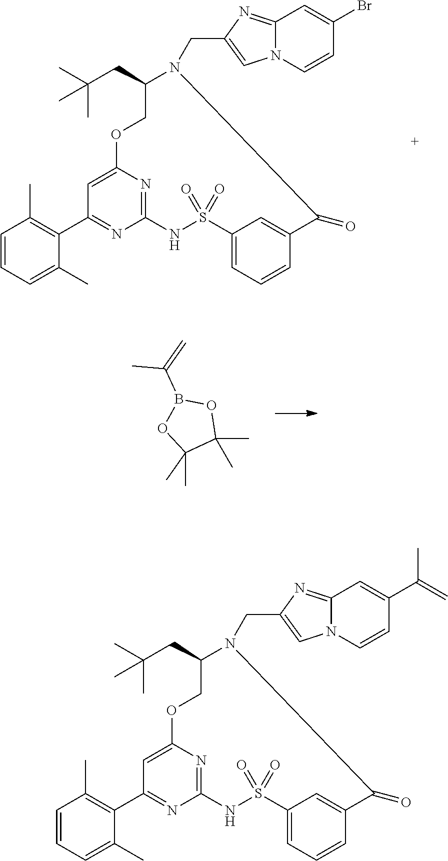 [SMILES: Cc1cccc(C)c1-c1cc2nc(n1)NS(=O)(=O)c1cccc(c1)C(=O)N(Cc1cn3ccc(Br)cc3n1)[C@H](CC(C)(C)C)CO2 ; C=C(C)B1OC(C)(C)C(C)(C)O1 ; C=C(C)c1ccn2cc(CN3C(=O)c4cccc(c4)S(=O)(=O)Nc4nc(cc(-c5c(C)cccc5C)n4)OC[C@H]3CC(C)(C)C)nc2c1]

In a 4 mL vial, to a stirred solution of (11R)-12-[(7-bromoimidazo[1,2-a]pyridin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (50 mg, 0.07106 mmol) in dioxane (0.7 mL) were added 2-isopropenyl-4,4,5,5-tetramethyl-1,3,2-dioxaborolane (30 mg, 0.1785 mmol), potassium carbonate (60 mg, 0.4341 mmol), and water (0.10 mL). The tube was sealed, sparged with nitrogen for 5 min, then Pd(dppf)Cl₂ (5 mg, 0.006833 mmol) was added, followed by addition of water (0.10 mL). The vial was purged with nitrogen for another 2 min, then capped under nitrogen and stirred at 80° C. for 14 h (overnight). The reaction mixture was allowed to cool to room temperature. Then water (0.2 mL), acetic acid (0.2 mL) and DMSO (0.5 mL) were added in that order. Purification by preparative reverse-phase HPLC (1-99% acetonitrile in water over 15 min, 5 mM HCl as modifier) furnished (11R)-6-(2,6-dimethylphenyl)-11-(2,2-dimethylpropyl)-12-[(7-isopropenylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (22 mg, 44%) as a white solid. ESI-MS m/z calc. 664.2832, found 665.2 (M+1)⁺; Retention time: 1.42 minutes. LC method A.

### Step 4: (11R)-6-(2,6-Dimethylphenyl)-11-(2,2-dimethylpropyl)-12-[(7-isopropylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound III-1

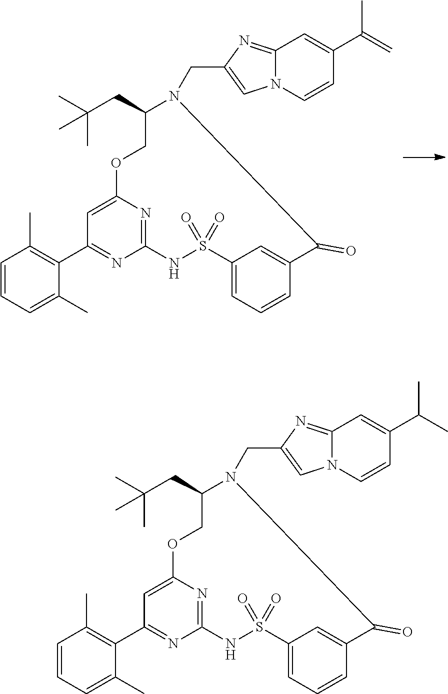 [SMILES: C=C(C)c1ccn2cc(CN3C(=O)c4cccc(c4)S(=O)(=O)Nc4nc(cc(-c5c(C)cccc5C)n4)OC[C@H]3CC(C)(C)C)nc2c1 ; Cc1cccc(C)c1-c1cc2nc(n1)NS(=O)(=O)c1cccc(c1)C(=O)N(Cc1cn3ccc(C(C)C)cc3n1)[C@H](CC(C)(C)C)CO2]

A stirred solution of (11R)-6-(2,6-dimethylphenyl)-11-(2,2-dimethylpropyl)-12-[(7-isopropenylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (22 mg, 0.03137 mmol) in methanol (3 mL) was added palladium (10 mg of 10% w/w, 0.009397 mmol) and sparged with nitrogen for 5 min. Then the reaction was stirred under hydrogen (balloon) for 1 h at ambient temperature. The mixture was sparged with nitrogen for 5 min and filtered over a pad of Celite. The filtrate was concentrated and purified by reverse-phase HPLC utilizing a gradient of 1-99% acetonitrile in 5 mM aq HCl to furnish (11R)-6-(2,6-dimethylphenyl)-11-(2,2-dimethylpropyl)-12-[(7-isopropylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (18 mg, 83%) as a white solid. ¹H NMR (400 MHz, CDCl₃) δ 8.47 (d, J=1.9 Hz, 1H), 8.36 (d, J=6.9 Hz, 1H), 8.11-8.00 (m, 2H), 7.87 (s, 1H), 7.62-7.52 (m, 2H), 7.22 (t, J=7.5 Hz, 2H), 7.04 (d, J=7.6 Hz, 2H), 6.13 (s, 1H), 5.19 (d, J=15.1 Hz, 1H), 5.13 (t, J=11.0 Hz, 1H), 5.01 (dd, J=10.3, 4.2 Hz, 1H), 4.94 (d, J=15.2 Hz, 1H), 4.12-4.01 (m, 1H), 3.11 (hept, J=6.9 Hz, 1H), 2.04-1.99 (m, 1H), 1.98 (s, 6H), 1.67 (d, J=15.5 Hz, 1H), 1.35 (d, J=6.8 Hz, 6H), 0.60 (s, 9H). ESI-MS m/z calc. 666.2988, found 667.9 (M+1)⁺; Retention time: 1.5 minutes. LC method A.

### Example 42: Preparation of Compound I-13

### Step 1: (1-Methylcyclobutyl)methyl 4-methylbenzenesulfonate

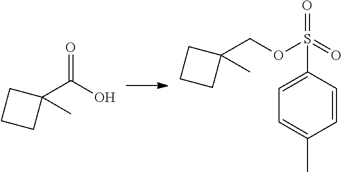

To a suspension of lithium aluminum hydride (11.5 g, 303.00 mmol) in ether (500 mL), 1-methylcyclobutanecarboxylic acid (25.000 g, 25 mL, 219.03 mmol) was slowly added at 0° C. The mixture was then heated at 35° C. for 4 h. The reaction mixture was cooled down to rt, and water (12 mL), NaOH 15% (12 mL) and water (36 mL) were successively and slowly added. The resulting mixture was filtered, and the precipitate was washed with ether. The filtrate was dried over sodium sulfate and concentrated in vacuo to furnish (1-methylcyclobutyl) methanol as a colorless liquid. To a solution of (1-methylcyclobutyl)methanol in DCM (500 mL), triethylamine (217.80 g, 300 mL, 2.1524 mol) and DMAP (3 g, 24.556 mmol) were added at 0° C. Tosyl chloride (95 g, 498.30 mmol) was added in several batches. The reaction mixture was slowly warmed up to rt and stirred for 2 days. Triethylamine (108.90 g, 150 mL, 1.0762 mol) and tosyl chloride (45 g, 236.04 mmol) were added and the reaction was stirred for 2 days. The reaction was treated with 1 N HCl (aq.) (500 mL) and extracted with DCM (3×300 mL). The combined organic layers were washed with saturated sodium bicarbonate (300 mL) and brine (100 mL), dried over anhydrous sodium sulfate, and concentrated under vacuum. The residue was purified by silica gel chromatography in 20% ethyl acetate in hexane to furnish (1-methylcyclobutyl)methyl 4-methylbenzenesulfonate (16.5 g, 28%) as a pale yellow liquid. ¹H NMR (400 MHz, Chloroform) δ 7.81 (d, J=8.3 Hz, 2H), 7.36 (d, J=8.1 Hz, 2H), 3.86 (s, 2H), 2.47 (s, 3H), 1.90-1.84 (m, J=7.3, 6.1, 3.8 Hz, 3H), 1.84-1.74 (m, 1H), 1.73-1.58 (m, 2H), 1.11 (s, 3H).

### Step 2: 2-(1-Methylcyclobutyl)acetonitrile

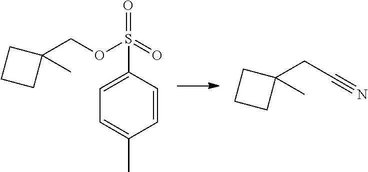

To a solution of (1-methylcyclobutyl)methyl 4-methylbenzenesulfonate (16.9 g, 66.445 mmol) in DMF (70 mL) was added KCN (13 g, 199.64 mmol). The reaction mixture was stirred at 80° C. for 16 h. After cooling to room temperature, the mixture was diluted with water (100 mL), and then extracted with ether (5×100 mL). The combined organic extracts were washed with water (50 mL) and brine (50 mL), dried over sodium sulfate, filtered, and concentrated in vacuo to give 2-(1-methylcyclobutyl)acetonitrile (5.1 g, 63%) as a pale yellow liquid. The crude product in ether was used in the next step without further purification. ¹H NMR (500 MHz, DMSO) δ 2.62 (s, 2H), 1.90-1.81 (m, 4H), 1.78-1.63 (m, 2H), 1.19 (s, 3H).

### Step 3: 2-(1-Methylcyclobutyl)acetaldehyde

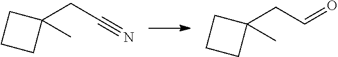

To a stirred solution of 2-(1-methylcyclobutyl)acetonitrile (4.5 g, 40.808 mmol) in DCM (60 mL) was added DIBAL in DCM (70 mL of 1 M, 70.000 mmol) at −78° C. over 2 minutes. The reaction mixture was stirred for 4 h, quenched with 20% AcOH (50 mL) at −78° C. The mixture was warmed to rt and saturated ammonium chloride (50 mL) was added and the reaction was stirred for 10 minutes. The organic layer was separated, washed with a sodium bicarbonate solution (50 mL), dried over sodium sulfate, filtered through a pad of silica, washed with ether and concentrated in vacuo. The residue was purified by silica gel chromatography in pentane to furnish 2-(1-methylcyclobutyl)acetaldehyde (1.6 g, 24%) as a clear liquid. ¹H NMR (500 MHz, DMSO-d6) δ 9.66 (s, 1H), 2.24 (s, 2H), 2.00-1.78 (m, 4H), 1.77-1.70 (m, 2H), 1.16 (s, 3H)

### Step 4: Methyl 2-(tert-butoxycarbonylamino)-4-(1-methylcyclobutyl)but-2-enoate

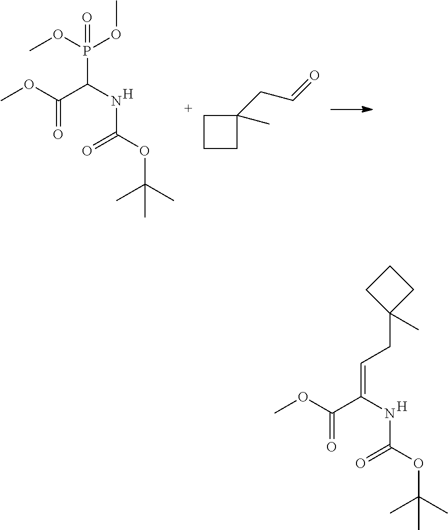

To a solution of methyl 2-(tert-butoxycarbonylamino)-2-dimethoxyphosphoryl-acetate (9.6 g, 31.328 mmol) and 2-(1-methylcyclobutyl)acetaldehyde (8.7 g, 25.595 mmol) in THE (105 mL) at −78° C. was added 1,1,3,3-Tetramethylguanidine (8.2620 g, 9 mL, 71.733 mmol) dropwise. The reaction mixture was warmed slowly to 0° C. over 3 h. The reaction mixture was quenched with ammonium chloride (150 mL) slowly at 0° C., diluted with water (150 mL). The two layers were separated, and the aqueous layer was extracted with ethyl acetate (3×150 mL). The combined organic layers were washed with brine (200 mL), dried over sodium sulfate, filtered, and concentrated in vacuo. The residue was purified by flash chromatography using 0-30% EtOAc in hexanes to provide methyl 2-(tert-butoxycarbonyl amino)-4-(1-methylcyclobutyl)but-2-enoate (4.72 g, 65%) as an off white solid. ESI-MS m z calc. 283.17834, found 284.3 (M+1)⁺; Retention time: 3.36 minutes; LC method E.

### Step 5: Methyl (2R)-2-(tert-butoxycarbonylamino)-4-(1-methylcyclobutyl)butanoate

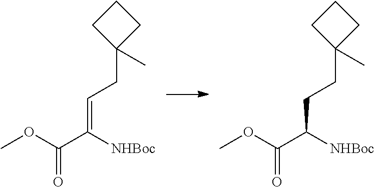

A solution of methyl 2-(tert-butoxycarbonylamino)-4-(1-methylcyclobutyl)but-2-enoate (3.65 g, 12.881 mmol) in EtOH (37.5 mL) and dioxane (12.5 mL) was purged with argon for 10 minutes. 1,2-bis[(2R,5R)-2,5-diethylphospholano]benzene(1,5-cyclooctadiene)rhodium(I) trifluoromethanesulfonate [CAS #136705-77-6](175.8 mg, 0.2433 mmol) was added to the reaction mixture. The reaction mixture was hydrogenated under 3.5 bar hydrogen pressure and at room temperature for 24 hours. The reaction mixture was filtered through silica gel and rinsed with EtOAc (40 mL). The filtrate was concentrated in vacuo. The residue was directly purified by silica-gel column chromatography using 0-40% ethyl acetate in hexanes to afford methyl (2R)-2-(tert-butoxycarbonylamino)-4-(1-methylcyclobutyl)butanoate (3.609 g, 98%) as a colorless oil. ESI-MS m/z calc. 285.194, found 286.2 (M+1)⁺; Retention time: 3.56 minutes; LC method E.

### Step 6: tert-Butyl N-[(1R)-1-(hydroxymethyl)-3-(1-methylcyclobutyl)propyl]carbamate

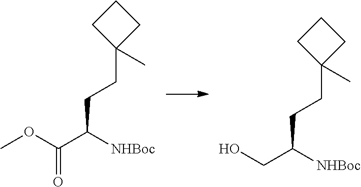

To a solution of methyl (2R)-2-(tert-butoxycarbonylamino)-4-(1-methylcyclobutyl)butanoate (3.6 g, 12.615 mmol) in THE (80 mL) at 0° C. was added dropwise LiBH₄ in THE (31.9 mL of 2 M, 63.800 mmol). The reaction mixture was stirred at room temperature for 3.5 h. The reaction mixture was cooled to 0° C., then quenched with saturated aqueous ammonium chloride (150 mL) and stirred vigorously until gas evolution ceased. The mixture was extracted with EtOAc (3×150 mL), washed with brine (200 mL), dried over sodium sulfate, filtered, and concentrated in vacuo. The residue was purified by flash chromatography using 0-60% EtOAc in hexanes to provide tert-butyl N-[(1R)-1-(hydroxymethyl)-3-(1-methylcyclobutyl)propyl]carbamate (3.18 g, 98%) as colorless gel. ESI-MS m/z calc. 257.1991, found 258.1 (M+1)⁺; LC method E.

### Step 7: (2R)-2-Amino-4-(1-methylcyclobutyl)butan-1-ol

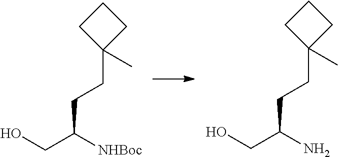 [SMILES: CC1(CC[C@H](CO)NC(=O)OC(C)(C)C)CCC1 ; CC1(CC[C@@H](N)CO)CCC1]

HCl in dioxane (58 mL of 4 M, 232.00 mmol) was added to tert-butyl N-[(1R)-1-(hydroxymethyl)-3-(1-methylcyclobutyl)propyl]carbamate (3.18 g, 11.143 mmol), and the reaction was stirred at room temperature for 3 h. The solvent was evaporated and dried under vacuum to afford crude (2R)-2-amino-4-(1-methylcyclobutyl)butan-1-ol (hydrochloride salt) (2.25 g, 99%) as pale yellow solid. ¹H NMR (500 MHz, DMSO-d6) δ 7.84 (s, 1H), 5.27 (t, J=5.0 Hz, 1H), 3.59 (dt, J=8.4, 4.3 Hz, 1H), 3.43 (dt, J=11.5, 5.4 Hz, 1H), 3.00 (s, 1H), 1.89-1.81 (m, 1H), 1.79-1.69 (m, 3H), 1.65-1.58 (m, 2H), 1.46-1.34 (m, 4H), 1.04 (s, 3H).

### Step 8: 3-[[4-[(2R)-2-Amino-4-(1-methylcyclobutyl)butoxy]-6-(2,6-dimethylphenyl)pyrimidin-2-yl]sulfamoyl]benzoic acid

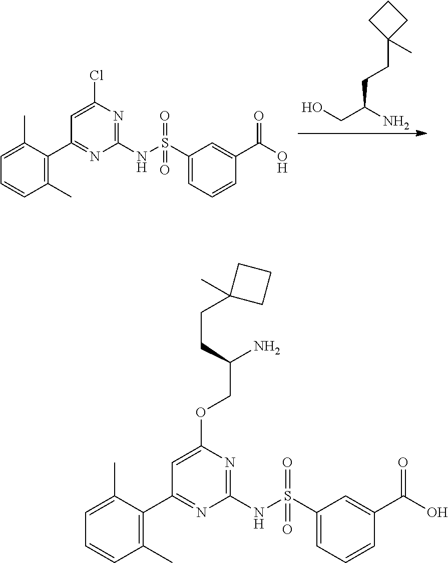 [SMILES: Cc1cccc(C)c1-c1cc(Cl)nc(NS(=O)(=O)c2cccc(C(=O)O)c2)n1 ; CC1(CC[C@@H](N)CO)CCC1 ; Cc1cccc(C)c1-c1cc(OC[C@H](N)CCC2(C)CCC2)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (1.4 g, 3.3504 mmol) and (2R)-2-amino-4-(1-methylcyclobutyl)butan-1-ol (hydrochloride salt) (623.8 mg, 3.0592 mmol) in anhydrous THE (20 mL) was added tBuONa (2.5 g, 26.014 mmol) at rt. The reaction mixture was stirred at rt for 3 hours. The reaction was quenched with 1 N HCl (45 mL), and then extracted with ethyl acetate (3×45 mL). The combined organic layers were washed with brine (45 mL), dried over anhydrous sodium sulfate, and concentrated under vacuum. The residue was purified by reverse phase HPLC (30 to 80% B, A=Water (0.1% HCl), B=Acetonitrile (0.1% HCl), 40 m, 60 mL/min). The desired product fractions were lyophilized. The material was triturated with 1:1 ethyl acetate and hexane (20 mL) to furnish 3-[[4-[(2R)-2-amino-4-(1-methylcyclobutyl)butoxy]-6-(2,6-dimethylphenyl)pyrimidin-2-yl]sulfamoyl]benzoic acid (hydrochloride salt) (1.4204 g, 78%) as a white powder. ¹H NMR (500 MHz, DMSO-d6) δ 13.23 (s, 1H), 8.46 (s, 1H), 8.32-8.05 (m, 5H), 7.69 (t, J=7.8 Hz, 1H), 7.26 (t, J=7.6 Hz, 1H), 7.12 (d, J=7.7 Hz, 2H), 6.32 (s, 1H), 4.41 (dd, J=12.0, 3.3 Hz, 1H), 4.30 (dd, J=11.8, 6.3 Hz, 1H), 3.50 (s, 1H), 2.00 (s, 6H), 1.87-1.81 (m, 1H), 1.76-1.68 (m, 3H), 1.64-1.59 (m, 2H), 1.55-1.47 (m, 3H), 1.40-1.33 (m, 1H), 1.04 (s, 3H). ESI-MS m/z calc. 538.225, found 539.2 (M+1)⁺; Retention time: 1.91 minutes; LC method H.

### Step 9: 3-{[4-(2,6-Dimethylphenyl)-6-[(2R)-4-(1-methylcyclobutyl)-2-({[6-(propan-2-yl)furo[2,3-b]pyrazin-2-yl]methyl}amino)butoxy]pyrimidin-2-yl]sulfamoyl}benzoic acid

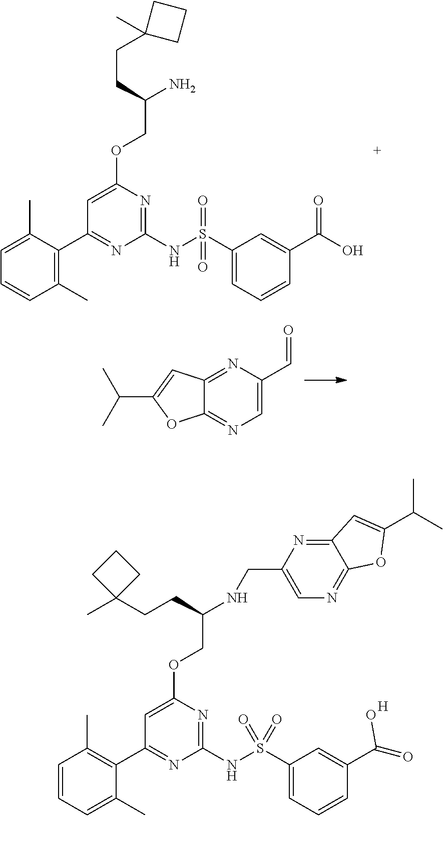

A 20 mL vial was charged under nitrogen with 3-[[4-[(2R)-2-amino-4-(1-methyl cyclobutyl)butoxy]-6-(2,6-dimethylphenyl)pyrimidin-2-yl]sulfamoyl]benzoic acid (hydrochloride salt) (301 mg, 0.5071 mmol), 6-isopropylfuro[2,3-b]pyrazine-2-carbaldehyde (97.5 mg, 0.5101 mmol), anhydrous DCM (2.5 mL), and acetic acid (0.03 mL, 0.5275 mmol). The mixture was cooled down in an ice bath. DIEA (0.2 mL, 1.148 mmol) was added, followed by sodium triacetoxyborohydride (Sodium salt) (657.1 mg, 3.100 mmol) and the reaction was vigorously stirred at 0° C. for 1 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-{[4-(2,6-dimethyl phenyl)-6-[(2R)-4-(1-methylcyclobutyl)-2-({[6-(propan-2-yl)furo[2,3-b]pyrazin-2-yl]methyl}amino)butoxy]pyrimidin-2-yl]sulfamoyl}benzoic acid (hydrochloride salt) (224.2 mg, 53%) as a white solid. ESI-MS m/z calc. 712.3043, found 713.8 (M+1)⁺; Retention time: 1.64 minutes; LC method A.

### Step 10: (11R)-6-(2,6-Dimethylphenyl)-12-[(6-isopropylfuro[2,3-b]pyrazin-2-yl)methyl]-11-[2-(1-methylcyclobutyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-13

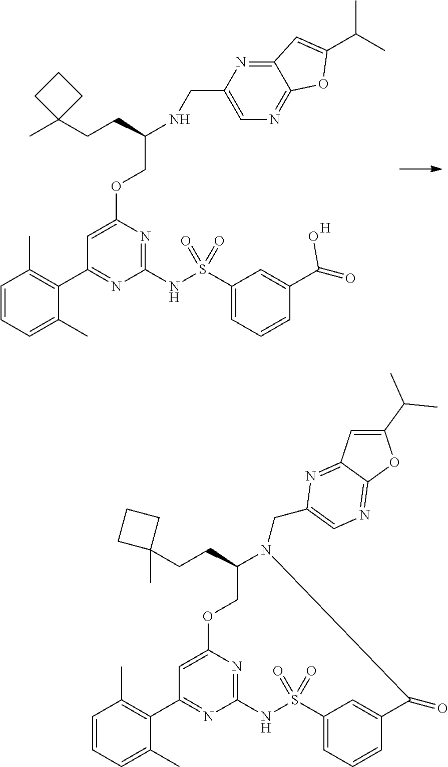

3-{[4-(2,6-Dimethylphenyl)-6-[(2R)-4-(1-methylcyclobutyl)-2-({[6-(propan-2-yl)furo[2,3-b]pyrazin-2-yl]methyl}amino)butoxy]pyrimidin-2-yl]sulfamoyl}benzoic acid (hydrochloride salt) (224.2 mg, 0.2693 mmol) was combined with CDMT (64.7 mg, 0.3685 mmol) in DMF (20 mL) and cooled to 0° C. N-methylmorpholine (0.21 mL, 1.910 mmol) was added by syringe and the reaction was stirred at 0° C. for 30 minutes. The ice bath was then removed and stirring was continued for an additional 16 hours at room temperature. The reaction mixture was then partitioned between 100 mL 1 M HCl and 100 mL ethyl acetate. The layers were separated, and the aqueous layer was extracted with an additional 100 mL ethyl acetate. The combined organic layers were washed 2×100 mL with brine, dried over sodium sulfate, filtered and concentrated. The crude product was diluted with DMSO (1 mL) and MeOH (1 mL), filtered, and purified by reverse phase HPLC (1-99% acetonitrile/5 mM aqueous HCl over 15 min) to give (11R)-6-(2,6-dimethylphenyl)-12-[(6-isopropylfuro[2,3-b]pyrazin-2-yl)methyl]-11-[2-(1-methylcyclobutyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (70.1 mg, 37%) as a white solid. ¹H NMR (400 MHz, CDCl₃) δ 8.63-8.60 (m, 1H), 8.43 (s, 1H), 8.18 (d, J=7.9 Hz, 1H), 7.85-7.80 (m, 1H), 7.67 (t, J=7.8 Hz, 1H), 7.18 (t, J=7.6 Hz, 1H), 6.98 (d, J=7.6 Hz, 2H), 6.61 (d, J=1.0 Hz, 1H), 6.14 (s, 1H), 5.39-5.32 (m, 2H), 4.33-4.21 (m, 2H), 4.08-3.99 (m, 1H), 3.21-3.12 (m, 1H), 2.05-1.81 (m, 7H), 1.78-1.60 (m, 7H), 1.40 (d, J=6.9 Hz, 6H), 1.38-1.30 (m, 1H), 1.04-0.99 (m, 4H). ESI-MS m/z calc. 694.29376, found 695.9 (M+1)⁺; Retention time: 2.3 minutes; LC method A.

### Example 43: Preparation of Compound I-15

### Step 1: Tetrahydropyran-2-ylmethyl 4-bromobenzenesulfonate

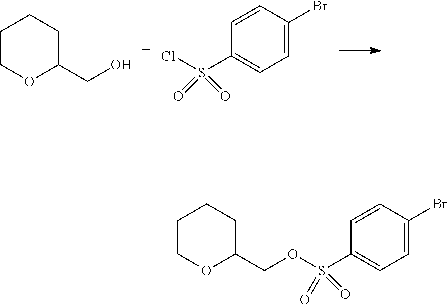 [SMILES: OCC1CCCCO1 ; O=S(=O)(Cl)c1ccc(Br)cc1 ; O=S(=O)(OCC1CCCCO1)c1ccc(Br)cc1]

To a stirred solution of tetrahydropyran-2-ylmethanol (10 g, 86.090 mmol) in anhydrous dichloromethane (300 mL) was added 4-bromobenzenesulfonyl chloride (24.1 g, 94.319 mmol). The reaction was then cooled down to 0° C. and 4-dimethylaminopyridine (1.1 g, 9.0040 mmol) and triethylamine (13.068 g, 18 mL, 129.14 mmol) were successively added. The reaction was stirred for 30 minutes at 0° C. then stirred overnight at room temperature. The reaction was cooled down to 0° C. then quenched by the addition of water (150 mL). The biphasic mixture was vigorously stirred for 15 minutes then the layers were separated and the organic layers was washed with an aqueous solution of iN hydrochloric acid (2×150 mL), then with an aqueous solution of iN sodium hydroxide (2×150 mL), brine (150 mL), dried over magnesium sulfate, filtered and concentrated under reduced pressure then dried under high vacuum to afford crude tetrahydropyran-2-ylmethyl 4-bromobenzenesulfonate (23.09 g, 80%) as a pale yellow oil which crystallized upon standing and could be used directly in the next Step without further purification. ¹H NMR (400 MHz, CDCl₃) δ 7.83-7.75 (m, 2H), 7.72-7.66 (m, 2H), 4.04-3.97 (m, 2H), 3.95-3.87 (m, 1H), 3.58-3.49 (m, 1H), 3.41-3.30 (m, 1H), 1.91-1.79 (m, 1H), 1.57-1.43 (m, 4H), 1.34-1.21 (m, 1H). ESI-MS m/z calc. 333.98746, found 335.0 (M+1)⁺; Retention time: 1.88 minutes; LC method I.

### Step 2: 2-(Iodomethyl)tetrahydropyran

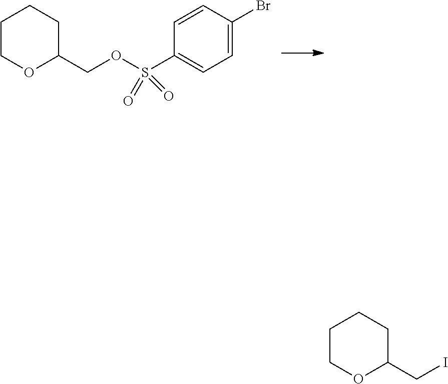 [SMILES: O=S(=O)(OCC1CCCCO1)c1ccc(Br)cc1 ; ICC1CCCCO1]

To a stirred solution of tetrahydropyran-2-ylmethyl 4-bromobenzenesulfonate (23.09 g, 68.812 mmol) in acetone (350 mL) was added sodium iodide (103 g, 687.15 mmol). The reaction was stirred at 45° C. for 48 hours after which time the solvent was evaporated under reduced pressure. The crude yellow solid was dissolved in water (500 mL) and the aqueous layer was extracted with heptanes (5×100 mL). The combined organic layers were washed with brine (300 mL), dried over magnesium sulfate, filtered and concentrated under reduced pressure to afford crude 2-(iodomethyl)tetrahydropyran (12.383 g, 76%) as a pale yellow oil which was used in the next Step without further purification. ¹H NMR (400 MHz, CDCl₃) δ 4.09-3.99 (m, 1H), 3.53-3.43 (m, 1H), 3.36-3.26 (m, 1H), 3.23-3.12 (m, 2H), 1.90-1.76 (m, 2H), 1.63-1.44 (m, 3H, overlapped with water), 1.37-1.24 (m, 1H).

### Step 3: Methyl (2R)-2-(benzyloxycarbonylamino)-3-tetrahydropyran-2-yl-propanoate

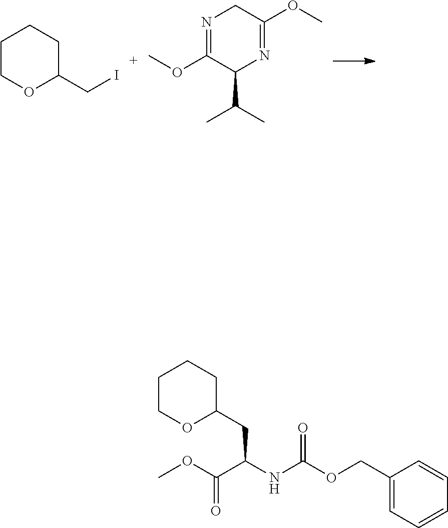

A stirred solution of (2S)-2-isopropyl-3,6-dimethoxy-2,5-dihydropyrazine (9.2820 g, 9.1 mL, 50.381 mmol) in anhydrous tetrahydrofuran (250 mL) under nitrogen atmosphere was cooled down to −78° C. Then a solution of n-butyllithium in hexanes (21 mL of 2.5 M, 52.500 mmol) was added and the bright yellow mixture was stirred at −78° C. for 30 minutes after which time a solution of 2-(iodomethyl)tetrahydropyran (12 g, 50.430 mmol) in anhydrous tetrahydrofuran (50 mL) was cannulated to the reaction mixture. The dark red solution was then stirred at −78° C. for 3 hours then allowed to reach room temperature over three days. The reaction was finally quenched by the addition of a saturated aqueous solution of ammonium chloride (250 mL). The layers were separated, and the aqueous layer was extracted with ethyl acetate (5×100 mL and the combined organic layers were washed with water (250 mL), and brine (250 mL), dried over magnesium sulfate, filtered and concentrated under reduced pressure to afford a light orange oil. The oil was then dissolved in acetonitrile (100 mL) and an aqueous solution hydrochloric acid (50 mL of 3 M, 150.00 mmol) was added. The reaction was stirred at room temperature for 2 hours after which time the solvent was removed under reduced pressure. The residue was then dissolved in a mixture of tetrahydrofuran (120 mL) and water (120 mL) and sodium bicarbonate (22 g, 261.88 mmol) was added portionwise over 20 minutes to the mixture. N-(Benzyloxycarbonyloxy)succinimide (31.5 g, 126.39 mmol) was added and the biphasic mixture was stirred overnight at room temperature. The layers were then separated, and the aqueous layer was extracted ethyl acetate (4×150 mL). The combined organic layers were washed with water (500 mL), brine (500 mL), dried over magnesium sulfate, filtered, and concentrated under reduced pressure to afford a bright orange oil. The crude was then purified by flash chromatography on silica gel (column: 330 g; gradient: 0 to 55% ethyl acetate in heptanes, 14 CV). The desired fractions were concentrated under reduced pressure to afford a pale-yellow oil. The oil was purified by reverse-phase chromatography on C₁₈ (column: 415 g C₁₈; gradient: 5 to 100% acetonitrile in acidic water containing 0.1% v/v of formic acid; 16 CV). The desired fractions were concentrated under reduced pressure and the remaining aqueous layer was neutralized using a saturated aqueous solution of sodium bicarbonate (50 mL). The aqueous layer was then extracted with ethyl acetate (4×250 mL) and the combined organic layers were washed with brine (500 mL), dried over magnesium sulfate, filtered and concentrated under reduced pressure to afford the (1:1) diastereomeric mixture of methyl (2R)-2-(benzyloxycarbonylamino)-3-tetrahydropyran-2-yl-propanoate (8.77 g, 54%) as a light-yellow oil that crystallized upon standing. ¹H NMR (400 MHz, CDCl₃) δ 7.51-7.29 (m, 5H), 6.07 (d, J=8.1 Hz, 0.5H), 5.71 (d, J=5.9 Hz, 0.5H), 5.20-5.06 (m, 2H), 4.57-4.49 (m, 0.5H), 4.44-4.34 (m, 0.5H), 4.01-3.92 (m, 0.5H), 3.90-3.83 (m, 0.5H), 3.78-3.61 (m, 3H), 3.42-3.26 (m, 2H), 1.99-1.86 (m, 2H), 1.85-1.73 (m, 1H), 1.57-1.39 (m, 4H), 1.36-1.21 (m, 1H). ESI-MS m/z calc. 321.15762, found 322.2 (M+1)⁺; Retention time: 1.8 minutes; LC method I.

### Step 4: Benzyl N-[(1R)-1-(hydroxymethyl)-2-tetrahydropyran-2-yl-ethyl]carbamate

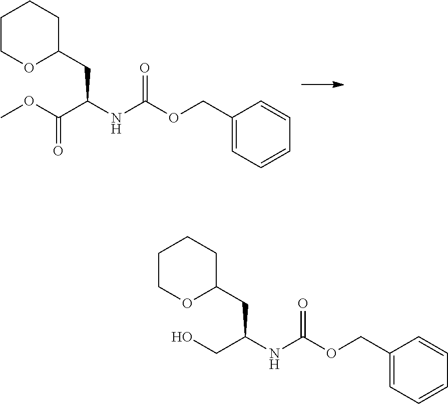 [SMILES: COC(=O)[C@@H](CC1CCCCO1)NC(=O)OCc1ccccc1 ; O=C(N[C@@H](CO)CC1CCCCO1)OCc1ccccc1]

A stirred solution of methyl (2R)-2-(benzyloxycarbonylamino)-3-tetrahydropyran-2-yl-propanoate (8.77 g, 27.262 mmol) in anhydrous tetrahydrofuran (100 mL) under nitrogen atmosphere was cooled down to 0° C. and lithium borohydride (1.65 g, 68.170 mmol) was added and the reaction was stirred at 0° C. for 2 hours after which time supplementary lithium borohydride (750 mg, 30.986 mmol) was added. The reaction was stirred for an additional 2 hours. The reaction was then quenched by the dropwise addition of a saturated aqueous solution of ammonium hydroxide (100 mL). Ethyl acetate (150 mL) was then added, and the biphasic mixture was stirred at 0° C. for 30 minutes. The layers were then separated, and the aqueous layer was extracted with ethyl acetate (5×150 mL). The combined organic layers were washed with brine (500 mL), dried over sodium sulfate, filtered and concentrated under reduced pressure to afford the crude diastereomeric mixture of benzyl N-[(1R)-1-(hydroxymethyl)-2-tetrahydropyran-2-yl-ethyl]carbamate (7.231 g, 89%) as a colorless oil which was directly used in the next step without further purification. ¹H NMR (400 MHz, CDCl₃) δ 7.41-7.29 (m, 5H), 5.62-5.46 (m, 1H), 5.17-5.05 (m, 2H), 4.01-3.92 (m, 1H), 3.90-3.80 (m, 1H), 3.77-3.59 (m, 2.5H), 3.48-3.33 (m, 2H), 2.90-2.71 (m, 0.5H), 1.91-1.42 (m, 7H, overlapped with water), 1.40-1.24 (m, 1H). ESI-MS m/z calc. 293.16272, found 294.2 (M+1)⁺; Retention time: 3.43 minutes; LC method J.

### Step 5: Benzyl N-[(1R)-1-(hydroxymethyl)-2-[(2R)-tetrahydropyran-2-yl]ethyl]carbamate and benzyl N-[(1R)-1-(hydroxymethyl)-2-[(2S)-tetrahydropyran-2-yl]ethyl]carbamate

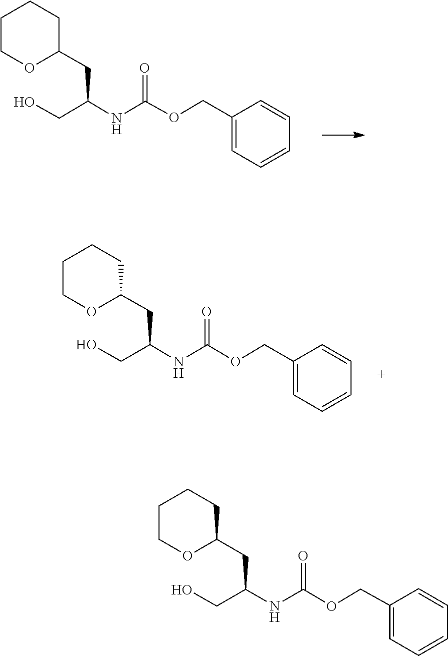 [SMILES: O=C(N[C@@H](CO)CC1CCCCO1)OCc1ccccc1 ; O=C(N[C@@H](CO)C[C@H]1CCCCO1)OCc1ccccc1 ; O=C(N[C@@H](CO)C[C@@H]1CCCCO1)OCc1ccccc1]

A diasteroeomeric mixture of benzyl N-[(1R)-1-(hydroxymethyl)-2-tetrahydropyran 2-yl-ethyl]carbamate (7.231 g, 24.205 mmol) was subjected to a SFC separation using the following conditions: a i-Amylose 3 column (250×21.2 mm), 5 μM column at 40° C., eluant: 10% methanol and 90% CO₂, flow rate: 75 mL/min, concentration: 62.3 mg/mL in methanol (no modifier), injection volume: 400 μL, pressure: 100 bars, wavelength: 210 nm. Evaporation of the solvents afforded the two major diastereoisomers as follow: SFC Peak 2: benzyl N-[(1R)-1-(hydroxymethyl)-2-[(2R)-tetrahydropyran-2-yl]ethyl]carbamate (2.99 g, 42%) as a pale yellow oil. ESI-MS m/z calc. 293.1627, found 294.2 (M+1)⁺; Retention time: 1.62 minutes, and SFC Peak 4: benzyl N-[(1R)-1-(hydroxymethyl)-2-[(2S)-tetrahydropyran-2-yl]ethyl]carbamate (2.19 g, 31%) as a pale yellow oil. ESI-MS m/z calc. 293.1627, found 294.2 (M+1)⁺; Retention time: 1.62 minutes, LC method I.

### Step 6: (2R)-2-Amino-3-[(2S)-tetrahydropyran-2-yl]propan-1-ol

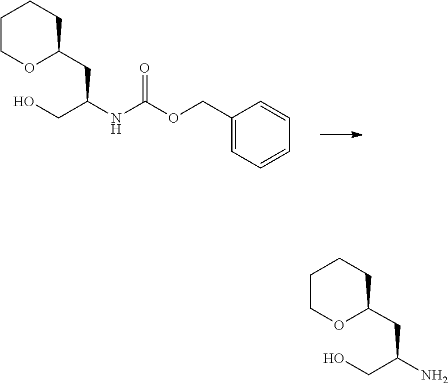 [SMILES: O=C(N[C@@H](CO)C[C@@H]1CCCCO1)OCc1ccccc1 ; N[C@@H](CO)C[C@@H]1CCCCO1]

Nitrogen was bubbled to a stirred solution of benzyl N-[(1R)-1-(hydroxymethyl)-2-[(2S)-tetrahydropyran-2-yl]ethyl]carbamate (2.19 g, 7.4578 mmol) in methanol (50 mL). Then palladium hydroxide on carbon (800 mg, 20% w/w, 1.1393 mmol) and nitrogen bubbling was kept on going for an additional 10 minutes. Hydrogen was then bubbled to the mixture for 10 minutes and the reaction was stirred overnight under hydrogen atmosphere. The reaction was then filtered on a pad of celite, washed with ethyl acetate (3×15 mL), concentrated under reduced pressure to afford crude (2R)-2-amino-3-[(2S)-tetrahydropyran-2-yl]propan-1-ol (1.14 g, 91%) as white crystals that were used in the next Step without further purification. ¹H NMR (400 MHz, DMSO-d6) δ 4.44 (br. s, 1H), 3.85-3.77 (m, 1H), 3.45-3.36 (m, 1H), 3.36-3.19 (m, 3H, overlapped with water), 3.08 (dd, J=10.3, 6.8 Hz, 1H), 2.81-2.71 (m, 1H), 1.81-1.69 (m, 1H), 1.63-1.34 (m, 6H), 1.21-1.05 (m, 2H). ESI-MS m/z calc. 159.12593, found 160.2 (M+1)⁺; Retention time: 0.37 minutes; LC method I.

### Step 7: 3-[[4-[(2R)-2-Amino-3-[(2S)-tetrahydropyran-2-yl]propoxy]-6-(2,6-dimethylphenyl)pyrimidin-2-yl]sulfamoyl]benzoic acid

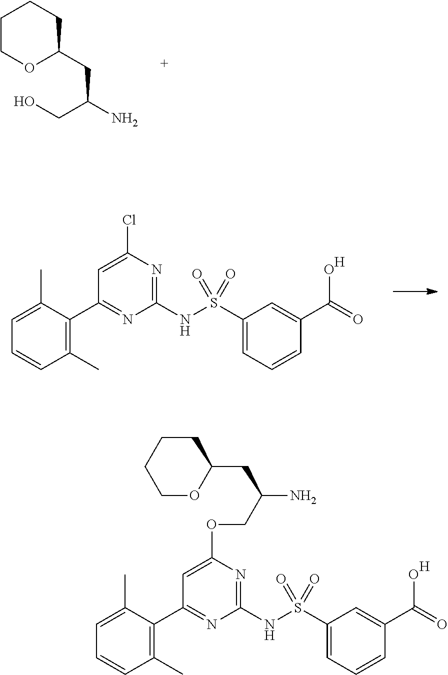

To a stirred solution of 3-[[4-chloro-6-(2,6-dimethylphenyl)pyrimidin-2-yl]sulfamoyl]benzoic acid (430 mg, 1.0290 mmol) in 2-methyltetrahydrofuran (12 mL) was added a solution of (2R)-2-amino-3-[(2S)-tetrahydropyran-2-yl]propan-1-ol (200 mg, 1.1933 mmol) in anhydrous DMF (2.5 mL). The mixture was then cooled down to 10-15° C. after which time sodium tert-butoxide (500 mg, 5.2027 mmol) was added. The reaction was stirred for 45 minutes after which time supplementary sodium tert-butoxide (250 mg, 2.6014 mmol) was added. The reaction was stirred for an additional hour and supplementary sodium tert-butoxide (250 mg, 2.6014 mmol) was added. The reaction was then stirred for 1.5 hours then cooled down to 0° C. then quenched by the addition of an aqueous solution of iN hydrochloric acid (25 mL). The mixture was vigorously stirred for 30 minutes, then the layers were separated, and the aqueous layer was extracted with 2-methyltetrahydrofuran (5×20 mL). The combined organic layers were washed with brine (75 mL), dried over magnesium sulfate, filtered and concentrated under reduced pressure to afford a pale yellow oil which was purified by reverse-phase chromatography on C₁₈ (column: 50 g: gradient: 5 to 100 methanol in water containing 0.1% v/v of hydrochloric acid; 15 CV). The desired fractions were concentrated under reduced pressure and the residue was co-evaporated with methanol (5×5 mL), then freeze dried to afford 3-[[4-[(2R)-2-amino-3-[(2S)-tetrahydropyran-2-yl]propoxy]-6-(2,6-dimethylphenyl)pyrimidin-2-yl]sulfamoyl]benzoic acid (hydrochloride salt) (238 mg, 39%) as a white powder. ¹H NMR (400 MHz, DMSO-d6) δ 13.37 (br. s, 1H), 8.45 (t, J=1.7 Hz, 1H), 8.24-8.03 (m, 5H), 7.70 (t, J=7.7 Hz, 1H), 7.26 (t, J=7.6 Hz, 1H), 7.16-7.08 (m, 2H), 6.30 (br. s, 1H), 4.40 (dd, J=11.7, 3.4 Hz, 1H), 4.22 (dd, J=11.6, 6.7 Hz, 1H, overlapped with water), 3.87-3.79 (m, 1H, overlapped with water), 3.69-3.60 (m, 1H), 3.52-3.42 (m, 1H), 3.41-3.32 (m, 1H), 2.00 (br. s, 6H), 1.83-1.66 (m, 3H), 1.56 (d, J=12.7 Hz, 1H), 1.50-1.36 (m, 3H), 1.30-1.16 (m, 1H). (1H missing, labile H). ESI-MS m/z calc. 540.2043, found 541.2 (M+1)⁺; Retention time: 2.4 minutes; LC method J.

### Step 8: 3-[[4-(2,6-Dimethylphenyl)-6-[(2R)-2-[(6-isopropyl-5-methyl-pyrrolo[2,3-b]pyrazin-3-yl)methylamino]-3-[(2S)-tetrahydropyran-2-yl]propoxy]pyrimidin-2-yl]sulfamoyl]benzoic acid

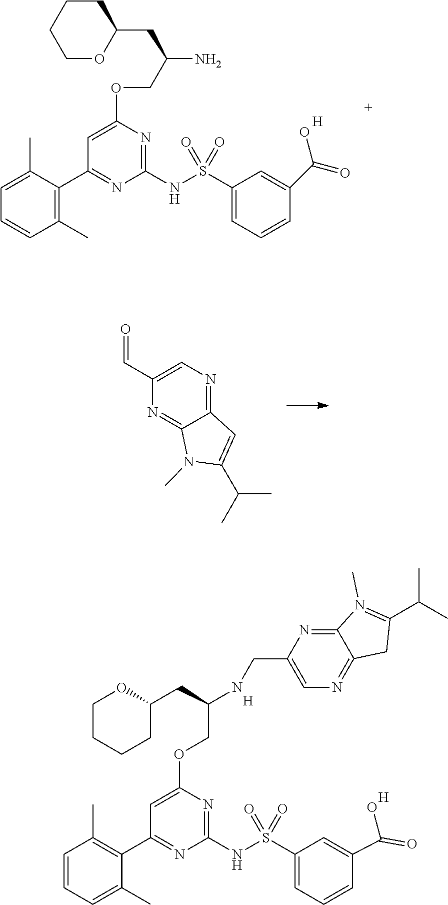

In a 4 mL vial, to a stirred mixture 3-[[4-[(2R)-2-amino-3-[(2S)-tetrahydropyran-2-yl]propoxy]-6-(2,6-dimethylphenyl)pyrimidin-2-yl]sulfamoyl]benzoic acid (hydrochloride salt) (35 mg, 0.06065 mmol) and 6-isopropyl-5-methyl-pyrrolo[2,3-b]pyrazine-3-carbaldehyde (12.4 mg, 0.06101 mmol) in anhydrous dcm (250 μL) were added glacial acetic acid (10 μL, 0.1758 mmol) and DIPEA (35 μL, 0.2009 mmol), in that order, at 0° C. under nitrogen. After 2 min, sodium triacetoxyborohydride (50 mg, 0.2359 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish 3-[[4-(2,6-dimethylphenyl)-6-[(2R)-2-[(6-isopropyl-5-methyl-pyrrolo[2,3-b]pyrazin-3-yl)methylamino]-3-[(2S)-tetrahydropyran-2-yl]propoxy]pyrimidin-2-yl]sulfamoyl]benzoic acid (hydrochloride salt) (33 mg, 71%) as a yellow solid. ESI-MS m z calc. 727.3152, found 728.9 (M+1)⁺; Retention time: 1.45 minutes; LC method A.

### Step 9: (11R)-6-(2,6-Dimethylphenyl)-12-[(6-isopropyl-5-methyl-pyrrolo[2,3-b]pyrazin-3-yl)methyl]-2,2-dioxo-1l-[[(2S)-tetrahydropyran-2-yl]methyl]-9-oxa-2λ⁶-thia-3,5,12,19-tetrazatricyclo[12.3.1.14,8]nonadeca-1(18),4(19),5,7,4,16-hexaen-13-one (Compound I-15

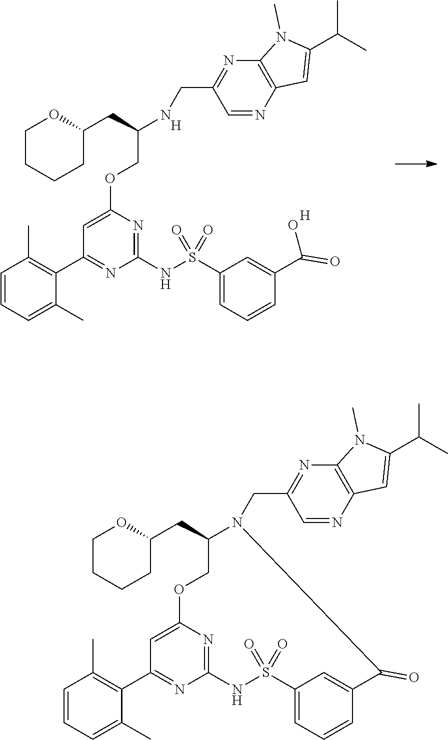

To a stirred solution of 3-[[4-(2,6-dimethylphenyl)-6-[(2R)-2-[(6-isopropyl-5-methyl-pyrrolo[2,3-b]pyrazin-3-yl)methylamino]-3-[(2S)-tetrahydropyran-2-yl]propoxy]pyrimidin-2-yl]sulfamoyl]benzoic acid (hydrochloride salt) (33 mg, 0.04317 mmol) in anhydrous DMF (1.6 mL) was added 2-chloro-4,6-dimethoxy-1,3,5-triazine (12 mg, 0.06835 mmol) (CDMT), followed by addition of 4-methylmorpholine (30 μL, 0.2729 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished product as a yellow solid. (11R)-6-(2,6-dimethylphenyl)-12-[(6-isopropyl-5-methyl-pyrrolo[2,3-b]pyrazin-3-yl)methyl]-2,2-dioxo-11-[[(2S)-tetrahydropyran-2-yl]methyl]-9-oxa-2λ⁶-thia-3,5,12,19-tetrazatricyclo[12.3.1.14,8]nonadeca-1(18),4(19),5,7,14,16-hexaen-13-one (20.50 mg, 66%) ¹H NMR (400 MHz, CDCl₃) δ 8.89 (t, J=1.8 Hz, 1H), 8.47 (s, 1H), 7.77 (dt, J=7.7, 1.4 Hz, 1H), 7.46 (t, J=7.8 Hz, 1H), 7.31-7.26 (m, 2H), 7.11 (d, J=7.6 Hz, 2H), 6.51 (s, 1H), 6.20 (s, 1H), 5.63-5.57 (m, 1H), 5.55 (d, J=15.4 Hz, 1H), 4.45-4.34 (m, 1H), 4.29 (d, J=15.7 Hz, 1H), 4.02 (t, J=11.4 Hz, 1H), 3.92 (s, 3H), 3.84 (d, J=11.2 Hz, 1H), 3.45 (dd, J=12.3, 10.1 Hz, 1H), 3.33-3.23 (m, 1H), 3.17 (hept, J=6.8 Hz, 1H), 2.14 (s, 6H), 1.90-1.77 (m, 3H), 1.66-1.51 (m, 3H), 1.51-1.43 (m, 1H), 1.40 (d, J=6.8 Hz, 3H), 1.37 (d, J=6.8 Hz, 3H), 1.29-1.19 (m, 1H). ESI-MS m/z calc. 709.3046, found 710.9 (M+1)⁺; Retention time: 1.72 minutes; LC method A.

### Example 44: Preparation of Compound I-18

### Step 1: (2R)-2-Amino-3-tetrahydrofuran-2-yl-propanoic acid

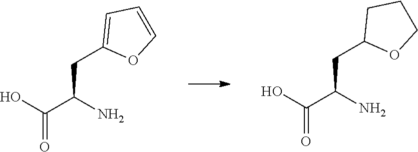 [SMILES: N[C@H](Cc1ccco1)C(=O)O ; N[C@H](CC1CCCO1)C(=O)O]

To a 600 mL stainless steel container were added (2R)-2-amino-3-(2-furyl)propanoic acid (4.5 g, 28.424 mmol), palladium 10% weight on carbon (477 mg, 10% w/w, 0.4482 mmol) and water (112.5 mL). The container was purged with nitrogen and inflated with hydrogen at 300 psi. The reaction was stirred at room temperature with mechanical stirring for 36 hours. Supplementary palladium 10% weight on carbon (150 mg, 10% w/w, 0.1410 mmol) was added to the reaction and the reaction was stirred for additional 21 hours. Then, the reaction solution was filtered twice with a celite pad and rinsed with water (2×225 mL). The filtrates were combined and evaporated under reduced pressure to afford an off-white residue which was freeze-dried to afford the crude diastereomeric mixture of (2R)-2-amino-3-tetrahydrofuran-2-yl-propanoic acid (4.601 g, 99%) as an off-white fluffy powder. The product could be used in the next Step without further purification. ¹H NMR (400 MHz, deuterium oxide) δ 4.16-4.08 (m, 0.5H), 4.04-3.94 (m, 0.5H), 3.89-3.80 (m, 1H), 3.79-3.71 (m, 1H), 3.56 (t, J=6.5 Hz, 1H), 3.21 (dd, J=18.6, 4.2 Hz, 0.5H), 3.14 (dd, J=18.8, 7.3 Hz, 0.5H), 2.62 (td, J=7.3, 2.4 Hz, 1H), 2.19-1.98 (m, 1.5H), 1.95-1.75 (m, 2H), 1.63-1.51 (m, 0.5H). (3H missing, labiles H). ESI-MS m/z calc. 159.08954, found 160.1 (M+1)⁺; Retention time: 0.26 minutes; LC method I.

### Step 2: (2R)-2-Amino-3-tetrahydrofuran-2-yl-propan-1-ol

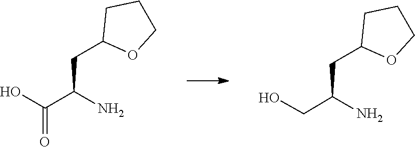 [SMILES: N[C@H](CC1CCCO1)C(=O)O ; N[C@@H](CO)CC1CCCO1]

To a 50 mL flame-dried flask were added (2R)-2-amino-3-tetrahydrofuran-2-yl-propanoic acid (1 g, 6.0936 mmol) and Me-THF (10.000 mL). A solution of Borane-THF complex in THF (13 mL of 1 M, 13.000 mmol) was injected dropwise over 15 minutes at 0° C. The reaction was kept at 0° C. for 0.5 h and run at room temperature for 21 hours. Then, another portion of solution of Borane-THF complex in THF (6.5 mL of 1 M, 6.5000 mmol) was injected at 0° C. The reaction was run overnight. Aqueous HCl (1 M) (30 mL of 1 M, 30.000 mmol) was used to quench the reaction at 0° C. The resulting solution was stirred for 60 minutes. Excess of THF was evaporated. 2-Methyltetrahydrofuran (25 mL) and iN NaOH (40 mL) solution were then added. The aqueous phase was extracted with 2-Me-THF (2×25 mL). The combined organic phase was washed with brine (15 mL), dried over sodium sulfate, filtered and concentrated, and dried under high vacuum to afford (2R)-2-amino-3-tetrahydrofuran-2-yl-propan-1-ol (hydrochloride salt) (1.107 g, 100%). ESI-MS m/z calc. 145.11028, found 146.02 (M+1)⁺; Retention time: 0.25 minutes; LC method I.

### Step 3: Benzyl N-[(1R)-1-(hydroxymethyl)-2-tetrahydrofuran-2-yl-ethyl]carbamate

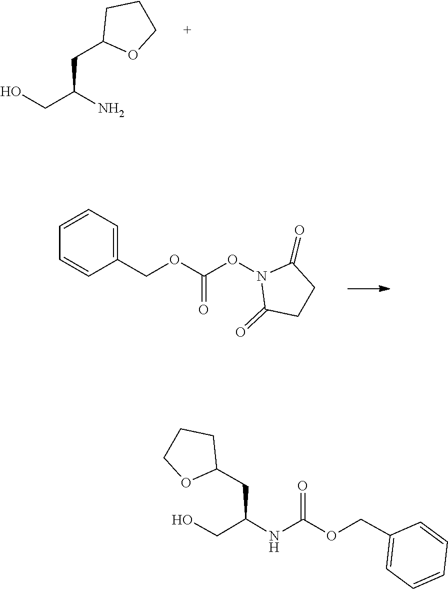 [SMILES: N[C@@H](CO)CC1CCCO1 ; O=C(OCc1ccccc1)ON1C(=O)CCC1=O ; O=C(N[C@@H](CO)CC1CCCO1)OCc1ccccc1]

To a 250 mL flask that containing the crude product of (2R)-2-amino-3-tetrahydrofuran-2-yl-propan-1-ol (hydrochloride salt) (1.08 g, 5.9452 mmol) were added benzyl (2,5-dioxopyrrolidin-1-yl) carbonate (1.63 g, 6.5404 mmol), potassium carbonate (2.7 g, 19.536 mmol), water (20 mL) and THF (20 mL). The reaction was stirred at room temperature for 5 hours and the layers were separated. The aqueous phase was extracted by CH₂Cl₂ (3×15 mL). The combined organic phase was washed with brine (15 mL), dried with sodium sulfate and evaporated under reduced pressure. Crude product (1.89 g) was purified by reverse-phase chromatography (column: C₁₈ 80 g; gradient: 5 to 100% acetonitrile in water containing 0.1% v/v of formic acid, 21 CV). The desired fractions were concentrated under reduced pressure, and acetonitrile was evaporated. The aqueous solution was neutralized with sodium bicarbonate and the pH value was between 8-9. The aqueous solution was extracted with ethyl acetate (3×50 mL), and the organic phase was separated, washed with brine (50 mL), dried with sodium sulfate, filtered and evaporated to afford benzyl N-[(1R)-1-(hydroxymethyl)-2-tetrahydrofuran-2-yl-ethyl]carbamate (1.204 g, 72%) as a white solid. ¹H NMR (400 MHz, CDCl₃) δ 7.39-7.27 (m, 5H), 5.67 (br. s, 1H), 5.17-5.04 (m, 2H), 3.99-3.82 (m, 3H), 3.82-3.62 (m, 3H), 2.11-1.97 (m, 2H), 1.97-1.81 (m, 2H), 1.71-1.57 (m, 2H), 1.55-1.40 (m, 1H). (1H missing, labile H) ESI-MS m/z calc. 279.14706, found 280.2 (M+1)⁺; Retention time: 1.53 minutes; LC method I.

### Step 4: Benzyl N-[(1R)-1-(hydroxymethyl)-2-tetrahydrofuran-2-yl-ethyl]carbamate, isomer 1, and benzyl N-[(1R)-1-(hydroxymethyl)-2-tetrahydrofuran-2-yl-ethyl]carbamate, isomer 2

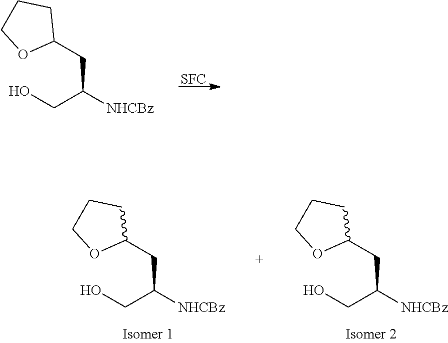

The diastereoisomeric mixture of benzyl N-[(1R)-1-(hydroxymethyl)-2-tetrahydrofuran-2-yl-ethyl]carbamate (1.2 g, 4.2917 mmol) was subjected to a SFC separation using the following conditions: a i-Amylose 3 column (250×21.2 mm), 5 M column at 40° C., eluant: 10% methanol and 90% CO₂, flow rate: 4 mL/min, concentration: 35 mg/mL in methanol (no modifier), injection volume: 400 μL, pressure: 100 bars, wavelength: 210 nm. Evaporation of the solvents afforded the two diastereoisomers as follow: Isomer 1, SFC peak 1: benzyl N-[(1R)-1-(hydroxymethyl)-2-tetrahydrofuran-2-yl-ethyl]carbamate (727 mg, 91%) as a colorless oil. ¹H NMR (400 MHz, CDCl₃) δ 7.38-7.27 (m, 5H), 5.67 (br. s, 1H), 5.18-5.04 (m, 2H), 3.98-3.83 (m, 4H), 3.80-3.74 (m, 1H), 3.72-3.63 (m, 2H), 2.09-1.98 (m, J=13.2, 4.4 Hz, 2H), 1.98-1.82 (m, 2H), 1.65-1.57 (m, 2H), 1.57-1.43 (m, J=11.8, 8.6 Hz, 1H). ESI-MS m/z calc. 279.1471, found 280.2 (M+1)⁺; Retention time: 3.183 minutes; and isomer 2, SFC peak 2: benzyl N-[(1R)-1-(hydroxymethyl)-2-tetrahydrofuran-2-yl-ethyl]carbamate (350 mg, 86%) as a colorless oil. ¹H NMR (400 MHz, CDCl₃) δ 7.38-7.27 (m, 5H), 5.54 (br. s, 1H), 5.10 (s, 2H), 3.96-3.71 (m, 5H), 3.69-3.63 (m, 1H), 2.99-2.78 (m, 1H), 2.06-1.99 (m, 1H), 1.99-1.80 (m, 3H), 1.72-1.63 (m, 1H), 1.59-1.41 (m, 1H). ESI-MS m/z calc. 279.1471, found 280.2 (M+1)⁺; Retention time: 3.192 minutes; LC method J.

### Step 5: (2R)-2-Amino-3-tetrahydrofuran-2-yl-propan-1-ol, isomer 1

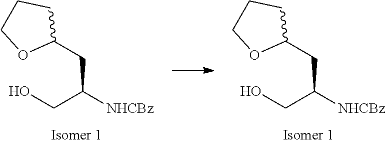

Nitrogen was bubbled to a stirred solution of benzyl N-[(1R)-1-(hydroxymethyl)-2-tetrahydrofuran-2-yl-ethyl]carbamate isomer 1 (720 mg, 2.5776 mmol) in anhydrous methanol (8 mL) for 15 minutes after which time palladium on carbon (275 mg, 10% w/w, 0.2584 mmol) was added. Nitrogen was then bubbled for 15 minutes, followed by hydrogen for 10 minutes. The reaction was then stirred overnight at room temperature under hydrogen atmosphere. The reaction was filtered on a pad of celite, washed with methanol (3×10 mL). The filtrate was concentrated under reduced pressure and IN HCl (3 mL) aqueous solution was added. The solution was then co-evaporated with methanol (5×5 mL) and the residue was freeze-dried to afford (2R)-2-amino-3-tetrahydrofuran-2-yl-propan-1-ol (hydrochloride salt) isomer 1(457 mg, 93%) as a pale yellow oil. ¹H NMR (400 MHz, DMSO-d6) δ 7.96 (br. s, 3H), 5.37-5.22 (m, 1H), 3.90-3.83 (m, 1H), 3.80-3.74 (m, 1H), 3.64-3.58 (m, 2H), 3.52-3.46 (m, 1H), 3.15 (br. s, 1H), 2.03-1.95 (m, 1H), 1.85-1.71 (m, 3H), 1.68-1.61 (m, 1H), 1.44-1.37 (m, 1H). ESI-MS m/z calc. 145.11028, found 146.2 (M+1)⁺; Retention time: 0.23 minutes; LC method I.

### Step 6: 3-[[4-[(2R)-2-Amino-3-tetrahydrofuran-2-yl-propoxy]-6-(2,6-dimethylphenyl)pyrimidin-2-yl]sulfamoyl]benzoic acid, isomer 1

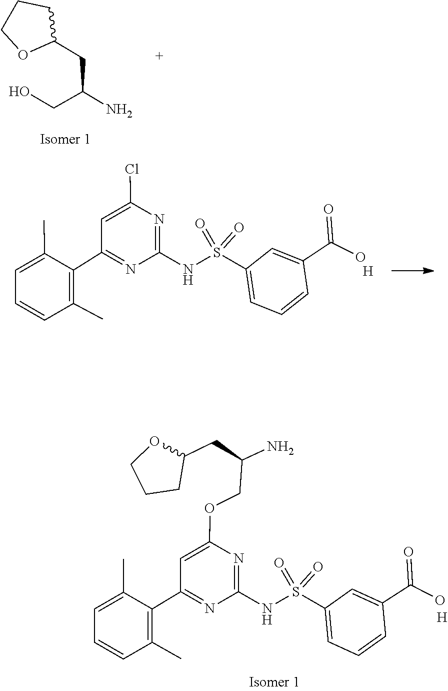

A solution of (2R)-2-amino-3-tetrahydrofuran-2-yl-propan-1-ol (hydrochloride salt) (457 mg, 2.3899 mmol) (isomer 1 from SFC peak 1) in anhydrous DMF (1.5 mL) was added to a solution of 3-[[4-chloro-6-(2,6-dimethylphenyl)pyrimidin-2-yl]sulfamoyl]benzoic acid (1.05 g, 2.5128 mmol) in 2-methyltetrahydrofuran (14 mL). The resulting solution was stirred at room temperature for 15 minutes, then cooled down to 10-15° C. and sodium tert-butoxide (920 mg, 9.5730 mmol) was added. The reaction was stirred at 10-15° C. for 1 h. Supplementary sodium tert-butoxide (575 mg, 5.9831 mmol) was added and the reaction was stirred for another 1 h. Again, supplementary sodium tert-butoxide (230 mg, 2.3933 mmol) was added and the reaction was stirred for 1.5 h. Then the reaction was cooled down to 0° C. and quenched by the addition of an aqueous solution of iN HCl (30 mL). The biphasic mixture was stirred for 20 minutes. The layers were then separated, and the aqueous layer was extracted with 2-methyltetrahydrofuran (7×20 mL). The combined organic layers were washed with brine (80 mL), dried over sodium sulfate, filtered and concentrated under reduced pressure to afford crude product as a yellow foam which was purified by reverse-phase chromatography on C₁₈ (column: 50 g C₁₈; gradient: 5 to 100% methanol in water containing 0.1% v/v of hydrochloric acid; 18 CV). The desired fractions were concentrated under reduced pressure and the remaining water was co-evaporated with methanol (5×20 mL), then with acetonitrile (4×10 mL) and freeze-dried to afford 3-[[4-[(2R)-2-amino-3-tetrahydrofuran-2-yl-propoxy]-6-(2,6-dimethylphenyl)pyrimidin-2-yl]sulfamoyl]benzoic acid (hydrochloride salt) (985 mg, 70%) (product of isomer 1 from SFC peak 1) as a white powder. ¹H NMR (400 MHz, DMSO-d6) δ 14.02-12.38 (br. s, 1H), 8.43 (t, J=1.7 Hz, 1H), 8.24-8.15 (br. s, 3H), 8.15-8.11 (m, 2H), 7.69 (t, J=7.8 Hz, 1H), 7.28-7.21 (m, 1H), 7.12 (d, J=7.6 Hz, 2H), 6.29 (s, 1H), 4.37-4.30 (m, 1H), 4.24 (dd, J=11.9, 6.7 Hz, 1H), 3.93-3.85 (m, 1H), 3.84-3.76 (m, 1H), 3.67-3.58 (m, 2H), 2.07-1.93 (m, 7H), 1.91-1.71 (m, 4H), 1.51-1.41 (m, 1H). (1H missing, labile proton). ESI-MS m/z calc. 526.1886, found 527.1 (M+1)⁺; Retention time: 2.14 minutes; LC method K.

### Step 7: 3-[[4-(2,6-Dimethylphenyl)-6-[(2R)-2-[(6-isopropylfuro[2,3-b]pyrazin-2-yl)methylamino]-3-tetrahydrofuran-2-yl-propoxy]pyrimidin-2-yl]sulfamoyl]benzoic acid, isomer 1

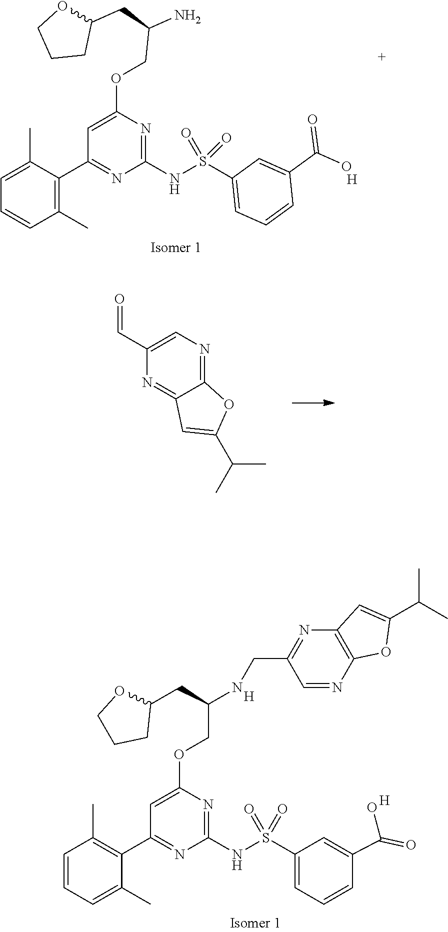

In a 4 mL vial, to a stirred mixture 3-[[4-[(2R)-2-amino-3-tetrahydrofuran-2-yl-propoxy]-6-(2,6-dimethylphenyl)pyrimidin-2-yl]sulfamoyl]benzoic acid (hydrochloride salt) (35 mg, 0.06216 mmol) (Isomer 1, SFC Peak-1) and 6-isopropylfuro[2,3-b]pyrazine-2-carbaldehyde (11.9 mg, 0.06257 mmol) in anhydrous dcm (250 μL) were added glacial acetic acid (10 μL, 0.1758 mmol) and DIPEA (35 μL, 0.2009 mmol), in that order, at 0° C. under nitrogen. After 2 min, sodium triacetoxyborohydride (50 mg, 0.2359 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish 3-[[4-(2,6-dimethylphenyl)-6-[(2R)-2-[(6-isopropylfuro[2,3-b]pyrazin-2-yl)methylamino]-3-tetrahydrofuran-2-yl-propoxy]pyrimidin-2-yl]sulfamoyl]benzoic acid isomer 1 (hydrochloride salt) (32 mg, 70%) as a white solid. ESI-MS m/z calc. 700.26794, found 701.9 (M+1)⁺; Retention time: 1.43 minutes; LC method A.

### Step 8: (11R)-6-(2,6-Dimethylphenyl)-12-[(6-isopropylfuro[2,3-b]pyrazin-2-yl)methyl]-2,2-dioxo-11-(tetrahydrofuran-2-ylmethyl)-9-oxa-2λ⁶-thia-3,5,12,19-tetrazatricyclo[12.3.1.14,8]nonadeca-1(18),4(19),5,7,4,16-hexaen-13-one, isomer 1 (Compound I-18

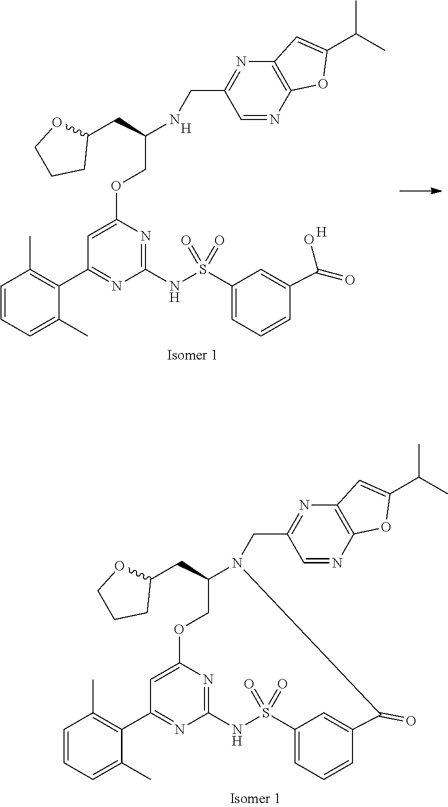

To a stirred solution of 3-[[4-(2,6-dimethylphenyl)-6-[(2R)-2-[(6-isopropylfuro[2,3-b]pyrazin-2-yl)methylamino]-3-tetrahydrofuran-2-yl-propoxy]pyrimidin-2-yl]sulfamoyl]benzoic acid (hydrochloride salt) (32 mg, 0.04340 mmol) (from Isomer 1, SFC Peak-1) in anhydrous DMF (1.5 mL) was added 2-chloro-4,6-dimethoxy-1,3,5-triazine (13 mg, 0.07404 mmol) (CDMT), followed by addition of 4-methylmorpholine (35 μL, 0.3183 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished product as a white solid. (11R)-6-(2,6-dimethylphenyl)-12-[(6-isopropylfuro[2,3-b]pyrazin-2-yl)methyl]-2,2-dioxo-11-(tetrahydrofuran-2-ylmethyl)-9-oxa-2λ⁶-thia-3,5,12,19-tetrazatricyclo[12.3.1.14,8]nonadeca-1(18),4(19),5,7,14,16-hexaen-13-one (16.50 mg, 55%) ¹H NMR (400 MHz, CDCl₃) δ 8.66 (t, J=1.8 Hz, 1H), 8.42 (s, 1H), 8.13 (d, J=7.9 Hz, 1H), 7.83 (dt, J=7.6, 1.4 Hz, 1H), 7.65 (t, J=7.8 Hz, 1H), 7.20 (t, J=7.6 Hz, 1H), 7.03 (d, J=7.6 Hz, 2H), 6.62 (d, J=1.0 Hz, 1H), 6.19 (s, 1H), 5.43-5.37 (m, 1H), 5.35 (d, J=14.9 Hz, 1H), 4.35 (t, J=11.4 Hz, 1H), 4.28 (d, J=14.9 Hz, 1H), 4.26-4.18 (m, 1H), 3.80-3.70 (m, 1H), 3.65-3.53 (m, 2H), 3.23-3.10 (m, 1H), 2.12-2.03 (m, 1H), 1.99 (s, 6H), 1.86-1.78 (m, 1H), 1.77-1.69 (m, 3H), 1.40 (d, J=7.0 Hz, 6H), 1.19-1.07 (m, 1H). ESI-MS m/z calc. 682.2573, found 683.9 (M+1)⁺; Retention time: 1.89 minutes; LC method A.

### Example 45: Preparation of Compound I-22

### Step 1: (2R)-2-Amino-3-tetrahydrofuran-2-yl-propan-1-ol, isomer 2

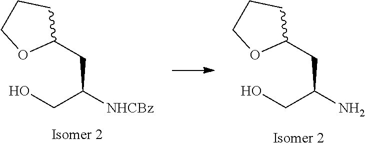

Nitrogen was bubbled to a stirred solution of benzyl N-[(1R)-1-(hydroxymethyl)-2-tetrahydrofuran-2-yl-ethyl]carbamate isomer 2 (350 mg, 1.2530 mmol) in anhydrous methanol (8 mL) for 15 minutes, after that palladium on carbon (135 mg, 10% w/w, 0.1269 mmol) was added. Nitrogen was then bubbled for another 10 minutes, followed by hydrogen bubbling for 10 minutes. The reaction was then stirred overnight at room temperature under hydrogen atmosphere. The reaction was filtered on a pad of celite, washed with methanol (3×10 mL). The filtrate was concentrated under reduced pressure and IN HCl (10 mL) aqueous solution was added. The solution was then co-evaporated with methanol (3×5 mL) and the residue was freeze-dried to afford (2R)-2-amino-3-tetrahydrofuran-2-yl-propan-1-ol isomer 2 (hydrochloride salt) (233 mg, 97%) as a yellow oil. ¹H NMR (400 MHz, DMSO-d6) δ 7.91 (br. s, 3H), 5.28 (br. s, 1H), 3.93-3.84 (m, 1H), 3.79-3.71 (m, 1H), 3.68-3.54 (m, 2H), 3.52-3.37 (m, 1H), 3.14 (br. s, 1H), 2.01-1.91 (m, 1H), 1.87-1.74 (m, 2H), 1.74-1.61 (m, 2H), 1.51-1.33 (m, 1H). ESI-MS m/z calc. 145.11028, found 146.2 (M+1)⁺; Retention time: 0.24 minutes; LC method I.

### Step 2: 3-[[4-[(2R)-2-Amino-3-tetrahydrofuran-2-yl-propoxy]-6-(2,6-dimethylphenyl)pyrimidin-2-yl]sulfamoyl]benzoic acid, isomer 2

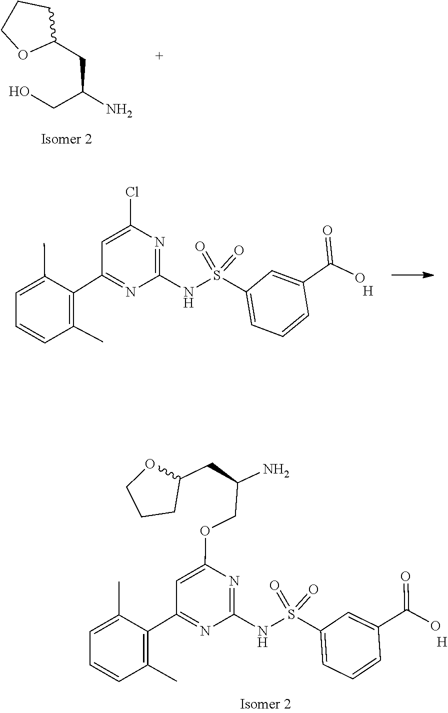

A solution of (2R)-2-amino-3-tetrahydrofuran-2-yl-propan-1-ol isomer 2 (hydrochloride salt) (230 mg, 1.2028 mmol) in anhydrous DMF (0.7 mL) was added to a solution of 3-[[4-chloro-6-(2,6-dimethylphenyl)pyrimidin-2-yl]sulfamoyl]benzoic acid (527 mg, 1.2612 mmol) in 2-methyltetrahydrofuran (7 mL). The resulting solution was stirred at room temperature for 15 minutes then cooled down to 10-15° C. and sodium tert-butoxide (463 mg, 4.8177 mmol) was added. The reaction was stirred at 10-15° C. for 45 minutes. Supplementary sodium tert-butoxide (116 mg, 1.2070 mmol) was added and the reaction was stirred for another 45 minutes. Again, supplementary sodium tert-butoxide (174 mg, 1.8105 mmol) was added and the reaction was stirred for 1 h. Then the reaction was cooled down to 0° C. and quenched by the addition of an aqueous solution of iN HCl (50 mL). The biphasic mixture was stirred for 30 minutes. The layers were then separated, and the aqueous layer was extracted with 2-methyltetrahydrofuran (5×20 mL). The combined organic layers were washed with brine (80 mL), dried over sodium sulfate, filtered and concentrated under reduced pressure to afford crude 3-[[4-[(2R)-2-amino-3-tetrahydrofuran-2-yl-propoxy]-6-(2,6-dimethylphenyl)pyrimidin-2-yl]sulfamoyl]benzoic acid isomer 2 (hydrochloride salt) (473 mg, 69%) ¹H NMR (400 MHz, DMSO-d6) δ 8.44 (t, J=1.6 Hz, 1H), 8.24-8.09 (m, 5H), 7.69 (t, J=7.8 Hz, 1H), 7.29-7.22 (m, 1H), 7.13 (d, J=7.6 Hz, 2H), 6.30 (br. s, 1H), 4.46-4.41 (m, 1H), 4.24 (dd, J=12.1 Hz, 1H), 3.97-3.87 (m, 1H), 3.82-3.73 (m, 1H), 3.68-3.56 (m, 2H), 2.09-1.91 (m, 7H), 1.90-1.71 (m, 4H), 1.53-1.43 (m, 1H). (2H missing, labile protons) ESI-MS m/z calc. 526.1886, found 527.1 (M+1)⁺; Retention time: 2.29 minutes as a yellow foam which was purified by reverse-phase chromatography on C₁₈ (column: 50 g C₁₈; gradient: 5 to 100% methanol in water containing 0.1% v/v of hydrochloric acid; 15 CV). The desired fractions were concentrated under reduced pressure and the remaining water was co-evaporated with methanol (5×20 mL), then with acetonitrile (4×10 mL) and freeze-dried to afford 3-[[4-[(2R)-2-amino-3-tetrahydrofuran-2-yl-propoxy]-6-(2,6-dimethylphenyl)pyrimidin-2-yl]sulfamoyl]benzoic acid (hydrochloride salt) (473 mg, 69%) as a white powder. ¹H NMR (400 MHz, DMSO-d6) δ 8.44 (t, J=1.6 Hz, 1H), 8.24-8.09 (m, 5H), 7.69 (t, J=7.8 Hz, 1H), 7.29-7.22 (m, 1H), 7.13 (d, J=7.6 Hz, 2H), 6.30 (br. s, 1H), 4.46-4.41 (m, 1H), 4.24 (dd, J=12.1 Hz, 1H), 3.97-3.87 (m, 1H), 3.82-3.73 (m, 1H), 3.68-3.56 (m, 2H), 2.09-1.91 (m, 7H), 1.90-1.71 (m, 4H), 1.53-1.43 (m, 1H). (2H missing, labile protons). ESI-MS m/z calc. 526.1886, found 527.1 (M+1)⁺; Retention time: 2.29 minutes; LC method J.

### Step 3: 3-[[4-(2,6-Dimethylphenyl)-6-[(2R)-2-[(6-isopropylfuro[2,3-b]pyrazin-2-yl)methylamino]-3-tetrahydrofuran-2-yl-propoxy]pyrimidin-2-yl]sulfamoyl]benzoic acid, isomer 2

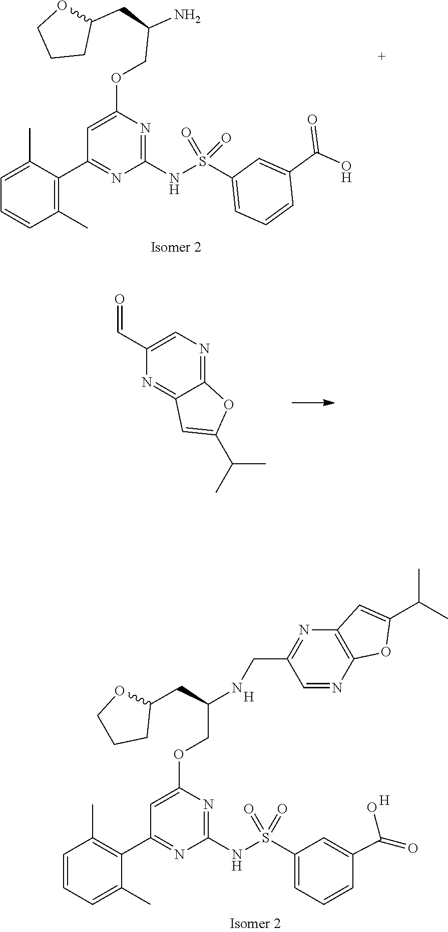

In a 4 mL vial, to a stirred mixture of 3-[[6-[(2R)-2-amino-3-tetrahydrofuran-2-yl-propoxy]-2-(2,6-dimethylphenyl)pyrimidin-4-yl]sulfamoyl]benzoic acid (hydrochloride salt) (35 mg, 0.06216 mmol) (Isomer 2, SFC Peak-2) and 6-isopropylfuro[2,3-b]pyrazine-2-carbaldehyde (11.9 mg, 0.06257 mmol) in anhydrous dcm (250 μL) were added glacial acetic acid (10 μL, 0.1758 mmol) and DIPEA (35 μL, 0.2009 mmol), in that order, at 0° C. under nitrogen. After 2 min, sodium triacetoxyborohydride (50 mg, 0.2359 mmol) was added to the yellow solution. The heterogeneous reaction was stirred at that temperature for 15 min. Then the reaction was quenched with aqueous 1M HCl (0.5 mL), MeOH (0.5 mL) and DMSO (0.5 mL) and purified by preparative reverse-phase HPLC [1-99% acetonitrile in water (containing 5 mM HCl) over 15 min] to furnish 3-[[4-(2,6-dimethylphenyl)-6-[(2R)-2-[(6-isopropylfuro[2,3-b]pyrazin-2-yl)methylamino]-3-tetrahydrofuran-2-yl-propoxy]pyrimidin-2-yl]sulfamoyl]benzoic acid isomer 2 (hydrochloride salt) (31 mg, 68%) as a white solid. ESI-MS m/z calc. 700.26794, found 701.9 (M+1)⁺; Retention time: 1.42 minutes; LC method A.

### Step 4: (11R)-6-(2,6-Dimethylphenyl)-12-[(6-isopropylfuro[2,3-b]pyrazin-2-yl)methyl]-2,2-dioxo-11-(tetrahydrofuran-2-ylmethyl)-9-oxa-2λ⁶-thia-3,5,12,19-tetrazatricyclo[12.3.1.14,8]nonadeca-1(18),4(19),5,7,4,16-hexaen-13-one, isomer 2 (Compound I-22

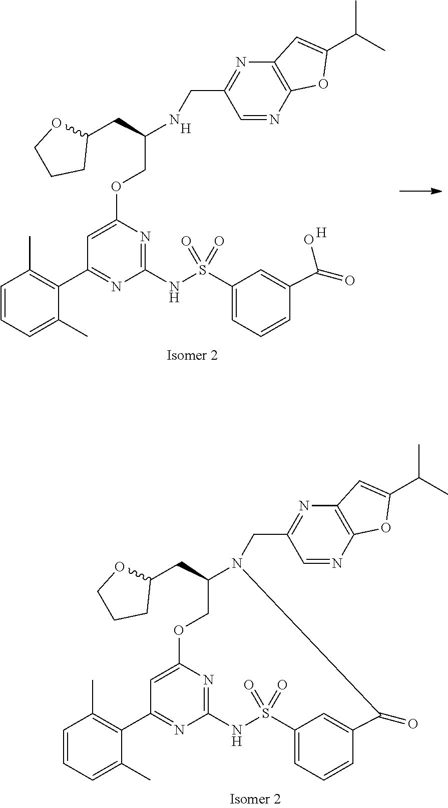

To a stirred solution of 3-[[4-(2,6-dimethylphenyl)-6-[(2R)-2-[(6-isopropylfuro[2,3-b]pyrazin-2-yl)methylamino]-3-tetrahydrofuran-2-yl-propoxy]pyrimidin-2-yl]sulfamoyl]benzoic acid (hydrochloride salt) (31 mg, 0.04205 mmol) (from Isomer 2, SFC Peak-2) in anhydrous DMF (1.5 mL) was added 2-chloro-4,6-dimethoxy-1,3,5-triazine (12 mg, 0.06835 mmol) (CDMT), followed by addition of 4-methylmorpholine (35 μL, 0.3183 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished product as a white solid. (11R)-6-(2,6-dimethylphenyl)-12-[(6-isopropylfuro[2,3-b]pyrazin-2-yl)methyl]-2,2-dioxo-11-(tetrahydrofuran-2-ylmethyl)-9-oxa-2λ⁶-thia-3,5,12,19-tetrazatricyclo[12.3.1.14,8]nonadeca-1(18),4(19),5,7,14,16-hexaen-13-one isomer 2 (18.5 mg, 63%) ¹H NMR (400 MHz, CDCl₃) δ 9.52 (s, 1H), 8.63 (t, J=1.8 Hz, 1H), 8.40 (s, 1H), 7.83-7.73 (m, 2H), 7.56 (t, J=7.8 Hz, 1H), 7.22 (t, J=7.6 Hz, 1H), 7.06 (d, J=7.6 Hz, 2H), 6.63 (d, J=1.0 Hz, 1H), 6.19 (s, 1H), 5.48-5.41 (m, 1H), 5.39 (d, J=14.8 Hz, 1H), 4.35-4.19 (m, 3H), 3.69-3.60 (m, 2H), 3.60-3.52 (m, 1H), 3.24-3.10 (m, 1H), 2.05 (s, 6H), 2.04-1.92 (m, 2H), 1.90-1.75 (m, 3H), 1.40 (d, J=7.0 Hz, 6H), 1.39-1.32 (m, 1H). ESI-MS m z calc. 682.2573, found 683.8 (M+1)⁺; Retention time: 1.91 minutes; LC method A.

### Example 46: Preparation of Compound I-23

### Step 1: 5-Chloro-3-(2-cyclopropylethynyl)-1-[(4-methoxyphenyl)methyl]pyrazin-2-one

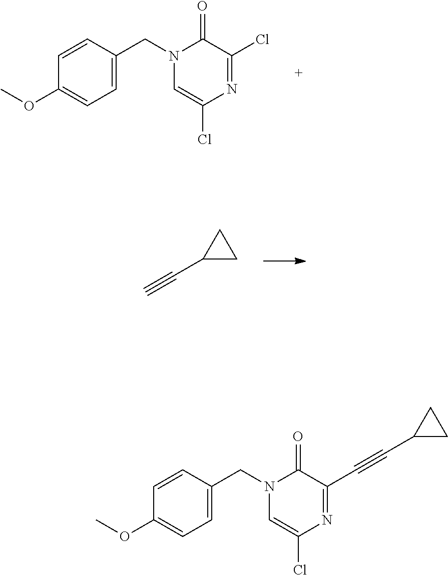

To a microwave vial was charged 3,5-dichloro-1-[(4-methoxyphenyl)methyl]pyrazin-2-one (2.95 g, 10.346 mmol), Bis(triphenylphosphine)palladium(II) dichloride (159.8 mg, 0.2277 mmol), CuI (98.8 mg, 0.5188 mmol) in DMF (20 mL) and TEA (20 mL). Then, ethynylcyclopropane (1.0309 g, 1.32 mL, 15.596 mmol) was added and sealed. The microwave vial was irradiated at 80° C. for 10 min. The reaction mixture was allowed to cool, diluted with DCM (100 mL) and washed with water (2×50 mL). The organic layer was washed with brine (50 mL), dried with sodium sulfate, filtered, and concentrated in vacuo. The residue was purified by silica gel chromatography using 0-50% ethyl acetate in hexanes to furnish 5-chloro-3-(2-cyclopropylethynyl)-1-[(4-methoxyphenyl)methyl]pyrazin-2-one (3.18 g, 98%) as a brown gel. ¹H NMR (500 MHz, DMSO-d6) δ 8.13 (s, 1H), 7.33 (d, J=8.8 Hz, 2H), 6.91 (d, J=8.7 Hz, 2H), 4.96 (s, 2H), 3.73 (s, 3H), 1.67-1.60 (m, 1H), 1.00-0.93 (m, 2H), 0.82-0.77 (m, 3H). ESI-MS m/z calc. 314.0822, found 315.1 (M+1)⁺; Retention time: 2.66 minutes; LC method E.

### Step 2: 2-Chloro-6-cyclopropyl-furo[2,3-b]pyrazine

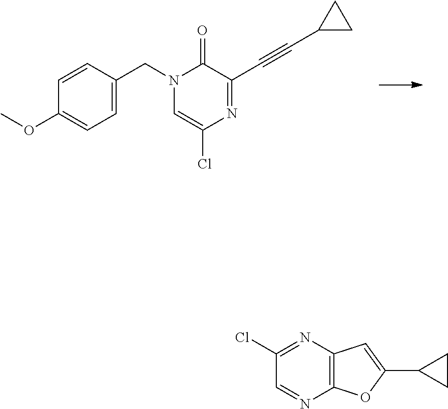 [SMILES: COc1ccc(Cn2cc(Cl)nc(C#CC3CC3)c2=O)cc1 ; Clc1cnc2oc(C3CC3)cc2n1]

To a solution of 5-chloro-3-(2-cyclopropylethynyl)-1-[(4-methoxyphenyl)methyl]pyrazin-2-one (20.73 g, 65.858 mmol) in DCM (440 mL), Silver trifluoromethanesulfonate (456.8 mg, 1.7779 mmol) and TFA (62.160 g, 42 mL, 545.15 mmol) were added and the reaction was stirred at room temperature for 40 minutes. The residue was concentrated in vacuo and purified by silica gel chromatography using 0-30% ethyl acetate in hexanes to give 2-chloro-6-cyclopropyl-furo[2,3-b]pyrazine (11.92 g, 86%) as a pale yellow solid. ESI-MS m/z calc. 194.02469, found 195.2 (M+1)⁺; Retention time: 2.84 minutes; LC method E.

### Step 3: Methyl 6-cyclopropyl-furo[2,3-b]pyrazine-2-carboxylate

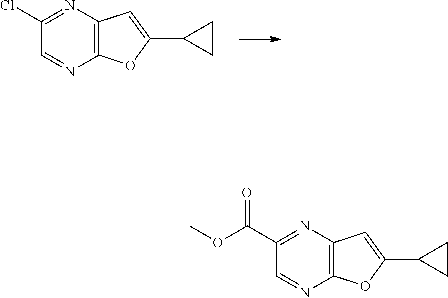 [SMILES: Clc1cnc2oc(C3CC3)cc2n1 ; COC(=O)c1cnc2oc(C3CC3)cc2n1]

A mixture of 2-chloro-6-cyclopropyl-furo[2,3-b]pyrazine (11.9 g, 58.088 mmol), Pd(dppf)Cl₂·DCM (3.3 g, 4.0410 mmol), TEA (68.970 g, 95 mL, 681.59 mmol) and MeOH (170 mL) in a steel bomb was purged with carbon monoxide three times. The reaction mixture was heated to 80° C. under 120 psi of carbon monoxide and stirred for 10 h. The reaction mixture was allowed to cool to rt. The reaction mixture was filtered through a pad of celite, washed with ethyl acetate (100 mL). The filtrate was concentrated in vacuo. The residue was purified by silica gel chromatography using 0-50% ethyl acetate in hexanes to afford methyl 6-cyclopropylfuro[2,3-b]pyrazine-2-carboxylate (7.8 g, 62%) as a pale yellow solid. ESI-MS m z calc. 218.06914, found 219.3 (M+1)⁺; Retention time: 2.25 minutes; LC method E.

### Step 4: 6-Cyclopropylfuro[2,3-b]pyrazine-2-carbaldehyde

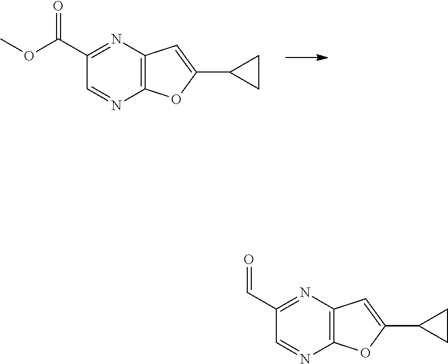 [SMILES: COC(=O)c1cnc2oc(C3CC3)cc2n1 ; O=Cc1cnc2oc(C3CC3)cc2n1]

To a stirred solution of methyl 6-cyclopropylfuro[2,3-b]pyrazine-2-carboxylate (7.8 g, 35.746 mmol) in DCM (275 mL) was added DIBAL in DCM (60 mL of 1 M, 60.000 mmol) at −78° C. The reaction mixture was stirred for 80 min, quenched with MeOH (10 mL)/water (10 mL) at −78° C. and the temperature was raised to rt. DCM (400 mL) was added and the solid mass was filtered. The cake was rinsed with DCM (100 mL). The filtrate was separated, washed with brine (2×200 ml), dried over sodium sulfate, filtered and concentrated in vacuo. The residue was purified by silica gel chromatography by using 0-50% ethyl acetate in hexanes to afford 6-cyclopropylfuro[2,3-b]pyrazine-2-carbaldehyde (5.4624 g, 79%) as a pale yellow solid. ¹H NMR (500 MHz, Chloroform-d) δ 10.18 (s, 1H), 8.80 (s, 1H), 6.73 (s, 1H), 2.22-2.16 (m, 1H), 1.26-1.19 (m, 4H). ESI-MS m/z calc. 188.05858, found 189.3 (M+1)⁺; Retention time: 1.74 minutes; LC method H.

### Step 5: Methyl (2R)-2-(benzyloxycarbonylamino)-3-isopropoxy-propanoate

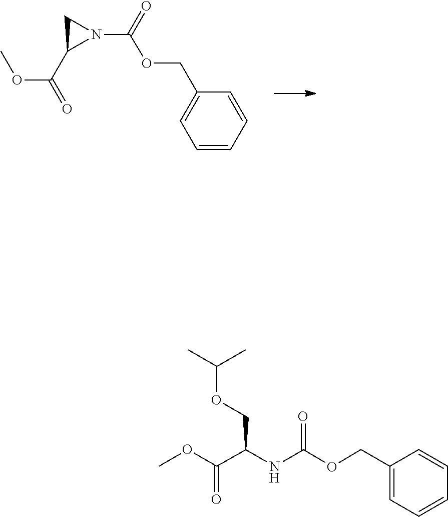 [SMILES: COC(=O)[C@H]1CN1C(=O)OCc1ccccc1 ; COC(=O)[C@@H](COC(C)C)NC(=O)OCc1ccccc1]

Boron trifluoride diethyletherate (770.50 mg, 0.67 mL, 5.4288 mmol) was added to a mixture of O1-benzyl O2-methyl (2R)-aziridine-1,2-dicarboxylate (7.748 g, 29.149 mmol) in iPrOH (200 mL) and DCM (100 mL). The reaction was stirred at room temperature for 24 h. The reaction mixture was diluted with DCM (20 mL) and the organic layer was washed with an aqueous saturated solution of sodium bicarbonate (10 mL), water (10 mL), dried over sodium sulfate, filtered, and evaporated under reduced pressure. The residue was purified by silica gel chromatography (column: 120 g, Combi flash Isco, 0-40% EtOAc in Heptanes) to afford methyl (2R)-2-(benzyloxycarbonylamino)-3-isopropoxy-propanoate (7.438 g, 59%) as a colorless oil. ESI-MS m/z calc. 295.14197, found 296.2 (M+1)⁺; Retention time: 1.79 minutes; LC method I.

### Step 6: Benzyl N-[(1S)-1-(hydroxymethyl)-2-isopropoxy-ethyl]carbamate

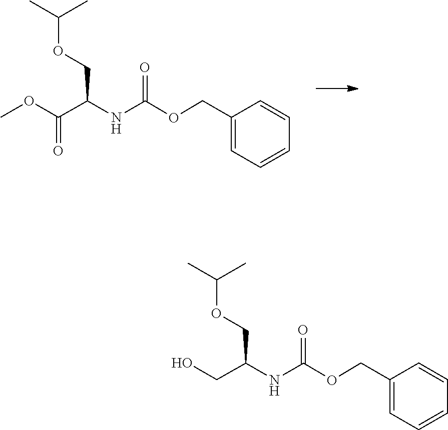 [SMILES: COC(=O)[C@@H](COC(C)C)NC(=O)OCc1ccccc1 ; CC(C)OC[C@H](CO)NC(=O)OCc1ccccc1]

Lithium borohydride in THE (50 mL of 2 M, 100.00 mmol) was added to a solution of methyl (2R)-2-(benzyloxycarbonylamino)-3-isopropoxy-propanoate (7.438 g, 17.126 mmol) in methanol (200 mL) at 0° C. The reaction was stirred for 5 minutes at 0° C. and then at room temperature for 16 h. An aqueous saturated solution of ammonium chloride (50 mL) was added and MeOH was removed under reduced pressure. The mixture was diluted with water (50 mL) and extracted with EtOAc (3×50 mL). The combined organic layers were dried with sodium sulfate, filtered and concentrated under reduced pressure. The crude was purified by reverse-phase chromatography (Column: C₁₈. Gradient: 5-100% methanol in water) to afford benzyl N-[(1S)-1-(hydroxymethyl)-2-isopropoxy-ethyl]carbamate (2.708 g, 52%) as a colorless oil. ESI-MS m/z calc. 267.14706, found 268.2 (M+1)⁺; Retention time: 1.61 minutes; LC method I.

### Step 7: (2S)-2-Amino-3-isopropoxy-propan-1-ol

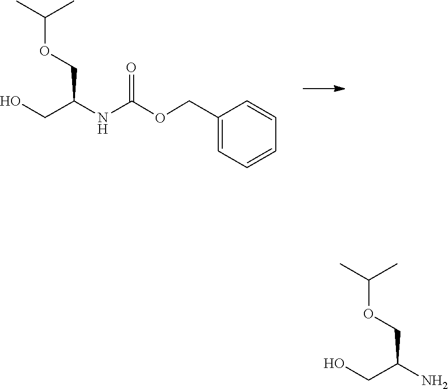

MeOH (25 mL) was added to benzyl N-[(1S)-1-(hydroxymethyl)-2-isopropoxy-ethyl]carbamate (2.708 g, 8.9044 mmol). The solution was degassed with nitrogen for 10 minutes and palladium on carbon (870 mg, 10% w/w, 0.8175 mmol) was added. The suspension was sparged with hydrogen for 5 minutes then stirred under a hydrogen atmosphere for 16 hours. The suspension was sparged with nitrogen for 10 minutes then filtered through a short pad of Celite which was washed with MeOH (2×50 mL). The solvent was removed under reduced pressure to afford crude (2S)-2-amino-3-isopropoxy-propan-1-ol (1.365 g, 100%) as a colorless oil. ¹H NMR (400 MHz, CDCl₃) δ 3.68-3.51 (m, 3H), 3.50-3.38 (m, 4H), 3.07 (quin, J=5.3 Hz, 1H), 2.40 (br. s., 4H), 1.16 (d, J=6.1 Hz, 6H). ESI-MS m/z calc. 133.11028, found 134.2 (M+1)⁺; Retention time: 1.27 minutes; LC method K.

### Step 8: 3-[[4-[(2R)-2-Amino-3-isopropoxy-propoxy]-6-(2,6-dimethylphenyl)pyrimidin-2-yl]sulfamoyl]benzoic acid

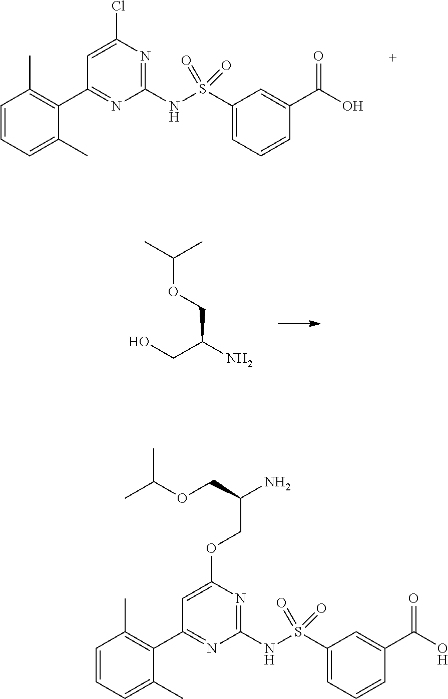

Sodium tert-butoxide (1.65 g, 17.169 mmol) was added in one portion to a solution of (2S)-2-amino-3-isopropoxy-propan-1-ol (536 mg, 3.3845 mmol) and 3-[[4-chloro-6-(2,6-dimethylphenyl)pyrimidin-2-yl]sulfamoyl]benzoic acid (1.17 g, 2.7999 mmol) in MeTHF (30 mL) and DMF (4 mL) at 15° C. The reaction was stirred at 15° C. for 2 h. The mixture was acidified with aqueous HCl 1 N (25 mL) and the phases were separated. The aqueous layer was extracted with EtOAc (3×25 mL). The combined organic layers were dried over sodium sulfate, filtered and concentrated under reduced pressure. The crude was purified by reverse-phase chromatography (Column: C₁₈. Gradient: 5-100% acetonitrile in acidic water (0.1% w/w of HCl)). The fractions containing the desired product were combined, evaporated to dryness and freeze-dried to afford 3-[[4-[(2R)-2-amino-3-isopropoxy-propoxy]-6-(2,6-dimethylphenyl)pyrimidin-2-yl]sulfamoyl]benzoic acid (hydrochloride salt) (1.116 g, 72%) as a white solid. ¹H NMR (400 MHz, DMSO-d6) δ 13.81-12.75 (br. s., 1H), 8.44 (t, J=1.6 Hz, 1H), 8.32-8.19 (m, 2H), 8.15 (dd, J=7.9, 1.3 Hz, 2H), 7.70 (t, J=7.7 Hz, 1H), 7.26 (t, J=7.6 Hz, 1H), 7.13 (d, J=7.6 Hz, 2H), 6.32 (br. s., 1H), 4.36 (d, J=4.6 Hz, 2H), 3.73-3.54 (m, 4H), 2.00 (br. s., 6H), 1.13 (dd, J=6.0, 1.3 Hz, 6H), (2H missing, labile protons). ESI-MS m/z calc. 514.1886, found 515.1 (M+1)⁺; Retention time: 2.36 minutes; LC method J.

### Step 9: 3-[[4-[(2R)-2-[(6-Cyclopropylfuro[2,3-b]pyrazin-2-yl)methylamino]-3-isopropoxy-propoxy]-6-(2,6-dimethylphenyl)pyrimidin-2-yl]sulfamoyl]benzoic acid

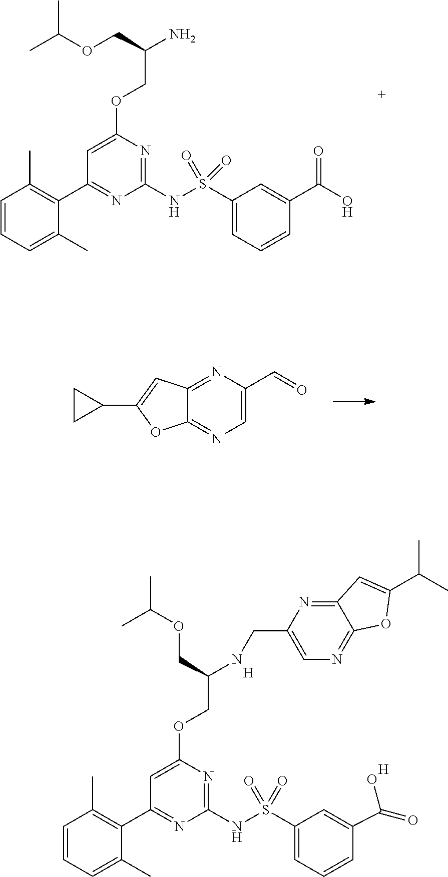

A 4 mL vial was charged under nitrogen with 3-[[4-[(2R)-2-amino-3-isopropoxy-propoxy]-6-(2,6-dimethylphenyl)pyrimidin-2-yl]sulfamoyl]benzoic acid (hydrochloride salt) (48.0 mg, 0.08641 mmol), 6-cyclopropylfuro[2,3-b]pyrazine-2-carbaldehyde (17.5 mg, 0.09290 mmol), anhydrous DCM (350 μL), acetic acid (11 μL, 0.1934 mmol) and DIEA (53 μL, 0.3043 mmol). The mixture was cooled down in an ice bath, sodium triacetoxyborohydride (64 mg, 0.3020 mmol) was added and the reaction was stirred for 1.5 h. The reaction was quenched with aqueous 1 M HCl (0.5 mL), MeOH (0.5 mL) and DMSO (0.5 mL), filtered and purified ion by reverse phase HPLC (1-99% acetonitrile/5 mM aqueous HCl over 30 min) to give as a white solid 3-[[4-[(2R)-2-[(6-cyclopropylfuro[2,3-b]pyrazin-2-yl)methylamino]-3-isopropoxy-propoxy]-6-(2,6-dimethylphenyl)pyrimidin-2-yl]sulfamoyl]benzoic acid (hydrochloride salt) (44.8 mg, 72%). ESI-MS m/z calc. 686.25226, found 687.2 (M+1)⁺; Retention time: 1.42 minutes; LC method A.

### Step 10: (11R)-12-[(6-Cyclopropylfuro[2,3-b]pyrazin-2-yl)methyl]-6-(2,6-dimethylphenyl)-11-(isopropoxymethyl)-2,2-dioxo-9-oxa-2λ⁶-thia-3,5,12,19-tetrazatricyclo[12.3.1.14,8]nonadeca-1(18),4(19),5,7,4,16-hexaen-13-one (Compound I-23

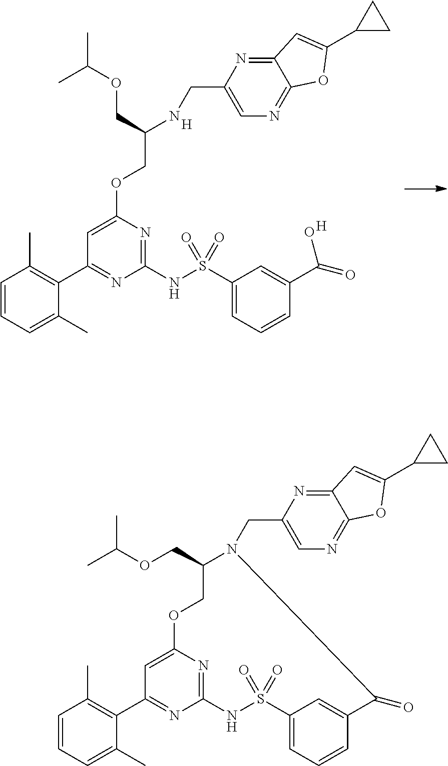

3-[[4-[(2R)-2-[(6-cyclopropylfuro[2,3-b]pyrazin-2-yl)methylamino]-3-isopropoxy-propoxy]-6-(2,6-dimethylphenyl)pyrimidin-2-yl]sulfamoyl]benzoic acid (hydrochloride salt) (44.0 mg, 0.06084 mmol) was combined under nitrogen with CDMT (29 mg, 0.1652 mmol) and DMF (1.5 mL). The solution was stirred at 0° C. 4-Methyl-morpholine (42 μL, 0.3820 mmol) was added and the mixture was stirred in the cooling bath that was allowed to warm to room temperature. After 14 h, the reaction was filtered and purified by reverse phase HPLC (1-99% acetonitrile/5 mM aqueous HCl over 30 min) to give as a white solid (11R)-12-[(6-cyclopropylfuro[2,3-b]pyrazin-2-yl)methyl]-6-(2,6-dimethylphenyl)-11-(isopropoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (21.5 mg, 52%), ¹H NMR (400 MHz, Chloroform-d) δ 9.52 (s, 1H), 8.67 (t, J=1.8 Hz, 1H), 8.31 (s, 1H), 7.89-7.74 (m, 2H), 7.59 (t, J=7.8 Hz, 1H), 7.23 (t, J=7.6 Hz, 1H), 7.06 (d, J=7.7 Hz, 2H), 6.62 (s, 1H), 6.22 (s, 1H), 5.41-5.28 (m, 2H), 4.57 (d, J=15.2 Hz, 1H), 4.45 (t, J=11.6 Hz, 1H), 4.38-4.27 (m, 1H), 3.65 (dd, J=10.7, 7.7 Hz, 1H), 3.58 (dd, J=10.7, 3.3 Hz, 1H), 3.52-3.38 (m, 1H), 2.18-2.07 (m, 1H), 2.04 (s, 6H), 1.21-1.12 (m, 4H), 1.08 (dd, J=20.9, 6.1 Hz, 6H). ESI-MS m/z calc. 668.2417, found 669.2 (M+1)⁺; Retention time: 2.0 minutes; LC method A.

### Example 47: Preparation of Compound III-2 and Compound III-3 and Compound III-5

### Step 1: 3-[[4-[(2R)-2-[(8-Bromoimidazo[1,2-a]pyridin-2-yl)methylamino]-4,4-dimethyl-pentoxy]-6-(2,6-dimethylphenyl)pyrimidin-2-yl]sulfamoyl]benzoic acid

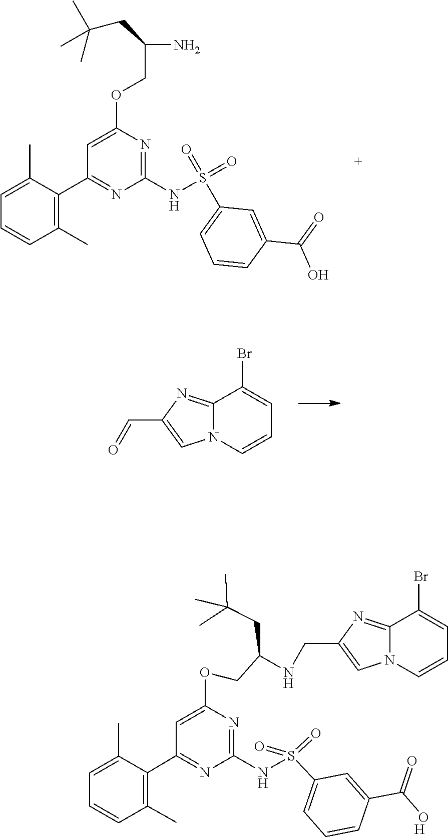

To a 0° C. solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (2.5 g, 4.4028 mmol) and 8-bromoimidazo[1,2-a]pyridine-2-carbaldehyde (937.5 mg, 3.9993 mmol) in dry dichloromethane (250 mL) was added sodium triacetoxyborohydride (3.75 g, 17.694 mmol). The mixture was stirred at 0° C. for 30 minutes. The reaction was then quenched at 0° C. by the addition of a 1 N aqueous hydrochloric acid solution (100 mL) and the mixture was stirred for 30 minutes at 0° C. The mixture was then concentrated in vacuo. The crude mixture was purified by reverse phase chromatography on a 275 g C₁₈ GOLD cartridge, eluting with a gradient of 5 to 100% of MeCN in acidic water (0.1% of hydrochloric acid in water) to afford after co-evaporation with MeCN (3×25 mL) 3-[[4-[(2R)-2-[(8-bromoimidazo[1,2-a]pyridin-2-yl)methylamino]-4,4-dimethyl-pentoxy]-6-(2,6-dimethylphenyl)pyrimidin-2-yl]sulfamoyl]benzoic acid (hydrochloride salt) (3.5 g, 67%) as a yellow solid. ESI-MS m/z calc. 720.173, found 721.0 (M+1)⁺; Retention time: 1.47 minutes; LC method I.

### Step 2: (11R)-12-[(8-Bromoimidazo[1,2-a]pyridin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound III-2

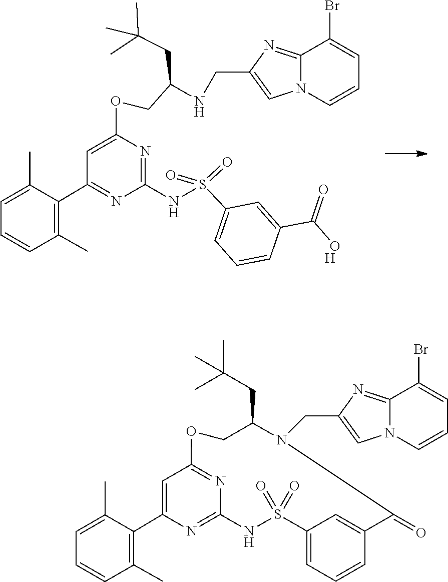

To a solution of 3-[[4-[(2R)-2-[(8-bromoimidazo[1,2-a]pyridin-2-yl)methylamino]-4,4-dimethyl-pentoxy]-6-(2,6-dimethylphenyl)pyrimidin-2-yl]sulfamoyl]benzoic acid (3.5 g, 3.0942 mmol) in DMF (350 mL) at 0° C. was added N-methylmorpholine (4.0480 g, 4.4 mL, 40.021 mmol) followed by 2-chloro-4,6-dimethoxy-1,3,5-triazine (1.1 g, 6.2652 mmol). The mixture was stirred at 0° C. for 15 minutes and then stirred at room temperature for 18 hours. Water (500 mL) and EtOAc (500 mL) were added, and the mixture was extracted with EtOAc (3×500 mL). The combined organic layers were washed with water (3×500 mL) and brine (5×500 mL), dried over sodium sulfate, filtered, and concentrated in vacuo. The crude mixture was purified twice by reverse phase chromatography on a 275 g C₁₈ GOLD cartridge, eluting with a gradient of 5 to 100% of MeCN in basic water (ammonium bicarbonate/ammonium hydroxide buffer pH=10) and then eluting with a gradient of 20 to 100% of MeCN in acidic water (0.1% v/v of formic acid in water) to afford after co-evaporation with water (2×50 mL) and lyophilization (11R)-12-[(8-bromoimidazo[1,2-a]pyridin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.3 g, 58%) as a pale yellow solid. ¹H NMR (400 MHz, CDCl₃) δ 8.61 (t, J=1.5 Hz, 1H), 8.12-8.07 (m, 2H), 7.88 (s, 1H), 7.87-7.83 (m, 1H), 7.66 (t, J=7.8 Hz, 1H), 7.46 (dd, J=7.3, 1.0 Hz, 1H), 7.21 (t, J=7.6 Hz, 1H), 7.05 (d, J=7.6 Hz, 2H), 6.70 (t, J=7.1 Hz, 1H), 6.24 (s, 1H), 5.25 (d, J=14.9 Hz, 1H), 5.08 (dd, J=11.2, 4.4 Hz, 1H), 4.56-4.42 (m, 2H), 4.20-4.10 (m, 1H), 2.03-1.93 (m, 7H), 1.54 (d, J=14.2 Hz, 1H, overlapped with water), 0.58 (s, 9H). 1 missing labile proton. ESI-MS m/z calc. 702.1624, found 703.1 (M+1)⁺; Retention time: 4.13 minutes; LC method J.

### Step 3: (11R)-6-(2,6-Dimethylphenyl)-11-(2,2-dimethylpropyl)-12-[(8-isopropenylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound III-3

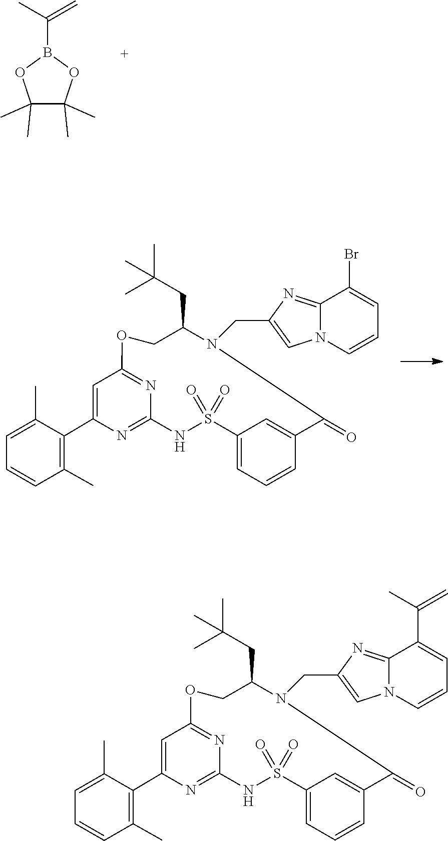

To a solution of (11R)-12-[(8-bromoimidazo[1,2-a]pyridin-2-yl)methyl]-6-(2,6-dimethylphenyl)-11-(2,2-dimethylpropyl)-2,2-dioxo-9-oxa-2λ⁶-thia-3,5,12,19-tetraza tricyclo[12.3.1.14,8]nonadeca-1(18),4(19),5,7,14,16-hexaen-13-one (550 mg, 0.7809 mmol) in dry dioxane (10 mL) was added 2-isopropenyl-4,4,5,5-tetramethyl-1,3,2-dioxaborolane (275 mg, 1.6365 mmol) followed by potassium carbonate (325 mg, 2.3516 mmol). Nitrogen was bubbled into the mixture for 15 minutes and then Pd(dppf)Cl₂ (130 mg, 0.1592 mmol) was added. Nitrogen was bubbled into the mixture for 10 minutes and the mixture was stirred at 100° C. for 18 hours. The mixture was then cooled down to room temperature and EtOAc (100 mL) was then added. The mixture was filtered on a pad of Celite and was concentrated in vacuo. Water (50 mL) and EtOAc (50 mL) were added, and the mixture was extracted with EtOAc (3×50 mL). The combined organic layers were washed with brine (100 mL), dried over sodium sulfate, filtered, and concentrated in vacuo. The crude mixture was purified twice by flash-chromatography on a silica gel cartridge (40 g Gold), eluting with a gradient of 0 to 100% of EtOAc in heptane and was then purified by reverse phase chromatography on a 100 g C₁₈GOLD cartridge, eluting with a gradient of 50 to 100% of MeOH in basic water (ammonium bicarbonate/ammonium hydroxide buffer pH=10). The fractions containing the desired product were combined and the organic solvent was evaporated. EtOAc (50 mL) was then added, and the mixture was extracted with EtOAc (3×50 mL). The combined organic layers were washed with brine (1×100 mL), dried over sodium sulfate, filtered and concentrated in vacuo to afford after lyophilization (11R)-6-(2,6-dimethylphenyl)-11-(2,2-dimethylpropyl)-12-[(8-isopropenyl imidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (205 mg, 37%) as a yellow solid. The crude (11R)-6-(2,6-dimethylphenyl)-11-(2,2-dimethylpropyl)-12-[(8-isopropenyl imidazo[1,2-a]pyridin-2-yl)methyl]-2,2-dioxo-9-oxa-2)⁶-thia-3,5,12,19-tetrazatricyclo[12.3.1.14,8]nonadeca-1(18),4(19),5,7,14,16-hexaen-13-one (200 mg, 0.2810 mmol) was further purified by reverse phase chromatography on a 50 g C₁₈ GOLD cartridge, eluting with a gradient of 50 to 100% of MeOH in basic water (ammonium bicarbonate/ammonium hydroxide buffer pH=10) to afford after co-evaporation with water (3×10 mL) and lyophilization (11R)-6-(2,6-dimethylphenyl)-11-(2,2-dimethylpropyl)-12-[(8-isopropenylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (169.5 mg, 88%) as a beige solid. ¹H NMR (400 MHz, CDCl₃) δ 8.75 (br. s, 1H), 8.61 (s, 1H), 8.06 (d, J=8.1 Hz, 1H), 8.02 (d, J=6.6 Hz, 1H), 7.86 (d, J=7.6 Hz, 1H), 7.77 (s, 1H), 7.64 (t, J=7.8 Hz, 1H), 7.21 (t, J=7.6 Hz, 1H), 7.16 (d, J=7.1 Hz, 1H), 7.05 (d, J=7.6 Hz, 2H), 6.79 (t, J=7.0 Hz, 1H), 6.39 (s, 1H), 6.24 (s, 1H), 5.52 (s, 1H), 5.24 (d, J=14.9 Hz, 1H), 5.13 (dd, J=11.4, 4.3 Hz, 1H), 4.54 (t, J=11.6 Hz, 1H), 4.43 (d, J=14.9 Hz, 1H), 4.21-4.12 (m, 1H), 2.33 (s, 3H), 2.04-1.89 (m, 7H), 1.54 (d, J=14.9 Hz, 1H, overlapped with water), 0.59 (s, 9H). ESI-MS m/z calc. 664.2832, found 665.3 (M+1)⁺; Retention time: 3.48 minutes; LC method J.

### Step 4: (11R)-6-(2,6-Dimethylphenyl)-11-(2,2-dimethylpropyl)-12-[(8-isopropylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound III-5

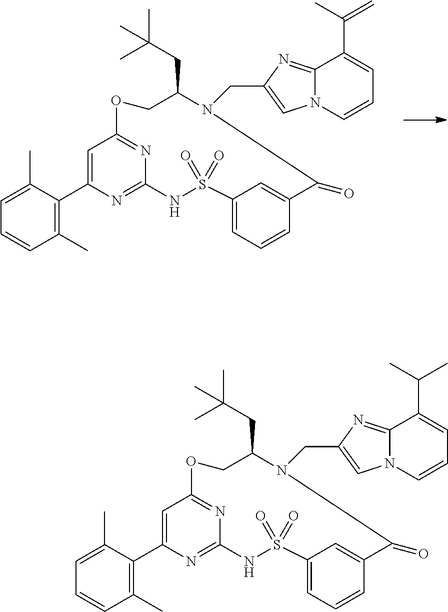 [SMILES: C=C(C)c1cccn2cc(CN3C(=O)c4cccc(c4)S(=O)(=O)Nc4nc(cc(-c5c(C)cccc5C)n4)OC[C@H]3CC(C)(C)C)nc12 ; Cc1cccc(C)c1-c1cc2nc(n1)NS(=O)(=O)c1cccc(c1)C(=O)N(Cc1cn3cccc(C(C)C)c3n1)[C@H](CC(C)(C)C)CO2]

Palladium (on carbon) (68 mg, 0.0319 mmol) was added into a 10 mL-flask and it was purged with nitrogen for 2 minutes. A solution of (11R)-6-(2,6-dimethylphenyl)-11-(2,2-dimethylpropyl)-12-[(8-isopropenylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (140 mg, 0.2034 mmol) in MeOH (3.5 mL) was then added into the flask. Hydrogen was bubbled into the suspension for 2 minutes and then the reaction mixture was stirred under 1 atm. of hydrogen for 60 minutes. Nitrogen was then bubbled into the mixture for 5 minutes. The reaction mixture was filtered on Celite, the pad was rinsed with EtOAc (10 mL) and MeOH (10 mL) and the filtrate was then concentrated in vacuo. The crude product was purified twice by reverse phase chromatography on a 30 g C₁₈ GOLD cartridge, eluting with a gradient of 50 to 100% of MeOH in acidic water (0.1% v/v of formic acid in water) to afford after co-evaporation with water (2×5 mL) and lyophilization (11R)-6-(2,6-dimethylphenyl)-11-(2,2-dimethylpropyl)-12-[(8-isopropylimidazo[1,2-a]pyridin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (48 mg, 35%) as a white solid. ¹H NMR (400 MHz, CDCl₃) δ 8.64 (br. s, 1H), 8.07 (d, J=7.1 Hz, 1H), 7.96 (br. s, 1H), 7.86 (d, J=7.6 Hz, 1H), 7.80-7.69 (m, 1H), 7.65 (t, J=7.8 Hz, 1H), 7.21 (t, J=7.6 Hz, 1H), 7.06 (d, J=7.6 Hz, 2H), 7.03-6.96 (m, 1H), 6.76 (br. s, 1H), 6.24 (s, 1H), 5.26 (d, J=14.9 Hz, 1H), 5.18-5.05 (m, 1H), 4.67-4.30 (m, 2H), 4.23-4.09 (m, 1H), 3.69 (br. s, 1H), 2.04-1.91 (m, 7H), 1.55 (d, J=10.3 Hz, 1H), 1.47-1.37 (m, 6H), 0.59 (s, 9H). 1 missing labile proton. ESI-MS m/z calc. 666.2988, found 667.3 (M+1)⁺; Retention time: 3.17 minutes; LC method J.

### Example 48: Preparation of Compound I-26

### Step 1: 5-Chloro-1-[(4-methoxyphenyl)methyl]-3-prop-1-ynyl-pyrazin-2-one

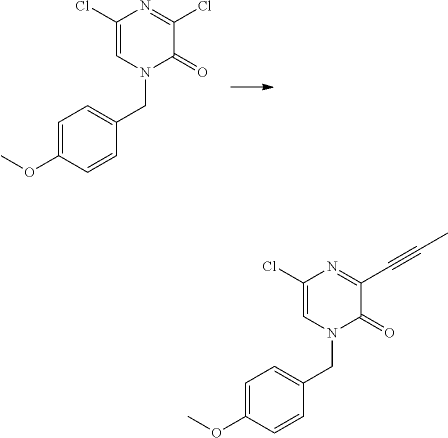 [SMILES: COc1ccc(Cn2cc(Cl)nc(Cl)c2=O)cc1 ; CC#Cc1nc(Cl)cn(Cc2ccc(OC)cc2)c1=O]

To a solution of 1-bromopropene (6.4 g, 52.903 mmol) in dry THE (150 mL) n-BuLi (50 mL of 1.5 M, 75.000 mmol) was added at −78° C. and the mixture was stirred for 20 min at −78° C. Then the mixture was warmed to 0° C. and water (2.00 g, 2 mL, 111.02 mmol) was added, and then the mixture was stirred for 5 min at this temperature. Then 3,5-dichloro-1-[(4-methoxyphenyl)methyl]pyrazin-2-one (5 g, 17.536 mmol), PdCl₂(PPh₃)₂ (1.5 g, 2.1371 mmol), CuI (400 mg, 2.1003 mmol) and TEA (30 mL) were added and the mixture was slowly warmed to room temperature and kept overnight. The dark mixture was diluted with 300 ml of ethyl acetate and filtered through a thin layer of silica gel. The filtrate was evaporated and the residue was purified by flash chromatography (ethyl acetate in hexane 10-40%) to give 5-chloro-1-[(4-methoxyphenyl)methyl]-3-prop-1-ynyl-pyrazin-2-one (3.8 g, 71%) as a brown solid. ESI-MS m/z calc. 288.06656, found 289.5 (M+1)⁺; Retention time: 4.4 minutes; LC method D.

### Step 2: 2-Chloro-7-iodo-6-methyl-furo[2,3-b]pyrazine

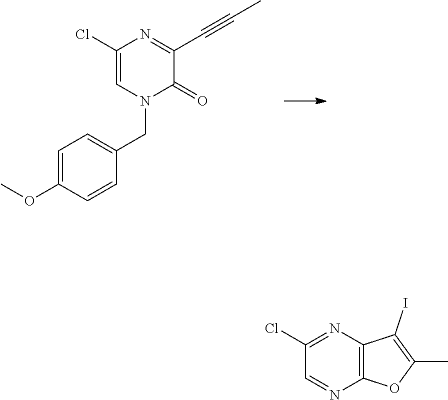 [SMILES: CC#Cc1nc(Cl)cn(Cc2ccc(OC)cc2)c1=O ; Cc1oc2ncc(Cl)nc2c1I]

To a solution of 5-chloro-1-[(4-methoxyphenyl)methyl]-3-prop-1-ynyl-pyrazin-2-one (3.8 g, 13.161 mmol) in dry DCM (150 mL), a solution of Iodine (3.51 g, 0.7120 mL, 13.829 mmol) in dry DCM (100 ml) was added over 30 min at room temperature and the resulting mixture was stirred at room temperature for an additional 30 min. After completion of reaction, the solvent was evaporated, and the residue was subjected to silica gel column chromatography (from 5% to 10% ethyl acetate in hexane). The collected fractions containing target substance were combined and evaporated. The residue was triturated with hexane (25 ml) for 2 hours. The precipitate was filtered and washed with hexane (25 ml) to afford 2-chloro-7-iodo-6-methyl-furo[2,3-b]pyrazine (2.91 g, 75%) as a pale yellow solid. ¹H NMR (500 MHz, CDCl₃) δ 8.18 (s, 1H), 2.67 (s, 3H). ESI-MS m/z calc. 293.90567, found 295.0 (M+1)⁺; Retention time: 2.34 minutes; LC method H.

### Step 3: 2-Chloro-7-cyclopropyl-6-methyl-furo[2,3-b]pyrazine

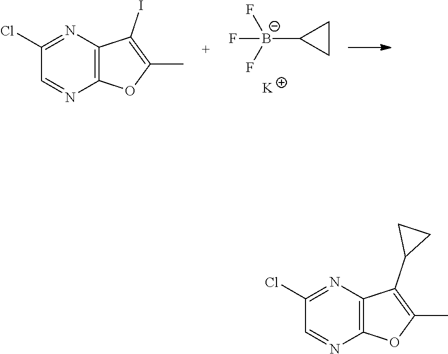

To a microwave vial was added 2-chloro-7-iodo-6-methyl-furo[2,3-b]pyrazine (1 g, 3.3959 mmol), potassium cyclopropyltrifluoroborate (500 mg, 3.3789 mmol), Cs₂CO₃ (3.4 g, 10.435 mmol), Xantphos (140 mg, 0.2420 mmol), PdOAc₂ (40 mg, 0.1782 mmol) and THE (24 mL). The reaction mixture was degassed for 15 minutes, and the reaction mixture was heated at 160° C. for 30 min. Then the reaction was cooled to rt and diluted with ethyl acetate (100 ml) and the organic phase was washed with water (100 ml), brine (100 ml), then dried (sodium sulfate), filtered and evaporated in vacuo. The residue was purified by silica gel chromatography (ethyl acetate 5-20% in hexanes) to give 2-chloro-7-cyclopropyl-6-methyl-furo[2,3-b]pyrazine (510 mg, 58%) as light yellow solid. ESI-MS m/z calc. 208.04034, found 209.4 (M+1)⁺; Retention time: 5.62 minutes; LC method D.

### Step 4: Methyl 7-cyclopropyl-6-methyl-furo[2,3-b]pyrazine-2-carboxylate

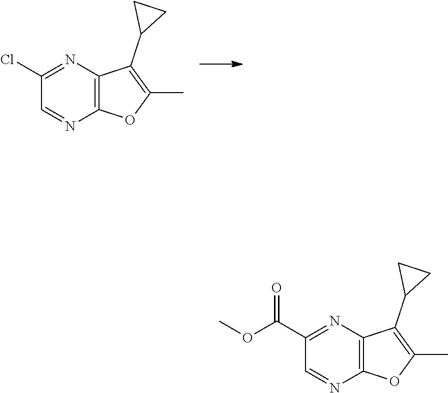

A mixture of 2-chloro-7-cyclopropyl-6-methyl-furo[2,3-b]pyrazine (710 mg, 3.4029 mmol) and Pd(dppf)Cl₂·DCM (270 mg, 0.3306 mmol) and TEA (1.6698 g, 2.3 mL, 16.502 mmol) in MeOH (40 mL) in a steel bomb equipped with mechanical stirrer was purged with carbon monoxide three times. The reaction mixture was heated to 100° C. with 120 psi of carbon monoxide and stirred overnight. The reaction mixture was allowed to cool to rt. The reaction mixture was filtered through a pad of celite, washed with ethyl acetate, and the filtrate was concentrated in vacuo. The residue was purified by silica gel chromatography in 50% ethyl acetate in hexanes to give as a pale yellow solid methyl 7-cyclopropyl-6-methyl-furo[2,3-b]pyrazine-2-carboxylate (610 mg, 73%). ¹H NMR (500 MHz, CDCl₃) δ 8.92 (s, 1H), 4.02 (s, 3H), 2.60 (s, 3H), 1.92-1.78 (m, 1H), 1.22-1.13 (m, 2H), 1.09-0.97 (m, 2H). ESI-MS m z calc. 232.0848, found 233.4 (M+1)⁺; Retention time: 4.63 minutes; LC method D.

### Step 5: 7-Cyclopropyl-6-methyl-furo[2,3-b]pyrazine-2-carbaldehyde

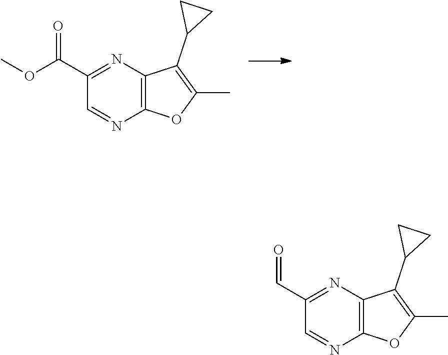

To a stirred solution of methyl 7-cyclopropyl-6-methyl-furo[2,3-b]pyrazine-2-carboxylate (600 mg, 2.5836 mmol) in DCM (20 mL) was added DIBAL in DCM (4.4 mL of 1 M, 4.4000 mmol) at −78° C. The reaction mixture was stirred for 2 h, quenched with MeOH (1 mL) and water (1 mL) and concentrated in vacuo. Then, DCM (20 mL) was added to the residue and the mass filtered through zeolite. The cake was washed with DCM (20 ml). The organic layer from the filtrate were separated, and the organic layer was washed with brine, dried over sodium sulfate, filtered and concentrated in vacuo. The resulting yellow oil was purified by silica gel chromatography (silica, 20 g, loaded with DCM, eluted with 70% ethyl acetate in hexanes). The desired product fractions were combined and concentrated in vacuo to give 7-cyclopropyl-6-methyl-furo[2,3-b]pyrazine-2-carbaldehyde (490 mg, 94%) as a pale yellow solid. ¹H NMR (500 MHz, CDCl₃) δ 10.17 (s, 1H), 8.80 (s, 1H), 2.62 (s, 3H), 1.88-1.80 (m, 1H), 1.31-1.25 (m, 2H), 1.07-1.00 (m, 2H). ESI-MS m/z calc. 202.07423, found 203.2 (M+1)⁺; Retention time: 2.05 minutes; LC method H.

### Step 6: 3-[[4-[(2R)-2-[(7-Cyclopropyl-6-methyl-furo[2,3-b]pyrazin-2-yl)methylamino]-3-isopropoxy-propoxy]-6-(2,6-dimethylphenyl)pyrimidin-2-yl]sulfamoyl]benzoic acid

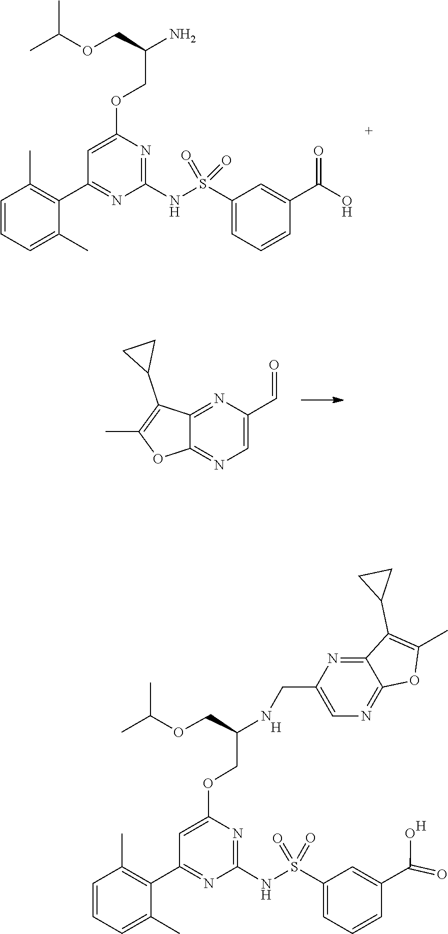

A 4 mL vial was charged under nitrogen with 3-[[4-[(2R)-2-amino-3-isopropoxy-propoxy]-6-(2,6-dimethylphenyl)pyrimidin-2-yl]sulfamoyl]benzoic acid (hydrochloride salt) (50 mg, 0.09074 mmol), 7-cyclopropyl-6-methyl-furo[2,3-b]pyrazine-2-carbaldehyde (19.3 mg, 0.09545 mmol), anhydrous DCM (0.65 mL), and acetic acid (0.006 mL, 0.1055 mmol). The mixture was cooled down in an ice bath. DIEA (0.03 mL, 0.1722 mmol) was added, followed by sodium triacetoxyborohydride (Sodium salt) (183.5 mg, 0.8658 mmol), and the reaction was vigorously stirred at 0° C. for 3 h. The reaction was quenched with 3 N aqueous HCl, diluted with MeOH and DMSO, and the resulting solution was filtered, and purified by reverse phase HPLC (1-99% acetonitrile/5 mM aqueous HCl over 15 min). The pure fractions were collected, extracted with EtOAc, dried over MgSO₄, filtered, and concentrated under reduced pressure to give 3-[[4-[(2R)-2-[(7-cyclopropyl-6-methyl-furo[2,3-b]pyrazin-2-yl)methylamino]-3-isopropoxy-propoxy]-6-(2,6-dimethylphenyl)pyrimidin-2-yl]sulfamoyl]benzoic acid (hydrochloride salt) (41.1 mg, 58%) as a white solid. ESI-MS m/z calc. 700.26794, found 701.4 (M+1)⁺; Retention time: 1.49 minutes. ESI-MS m/z calc. 700.26794, found 701.4 (M+1)⁺; Retention time: 1.49 minutes; LC method A.

### Step 7: (11R)-12-[(7-Cyclopropyl-6-methyl-furo[2,3-b]pyrazin-2-yl)methyl]-6-(2,6-dimethylphenyl)-11-(isopropoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-26

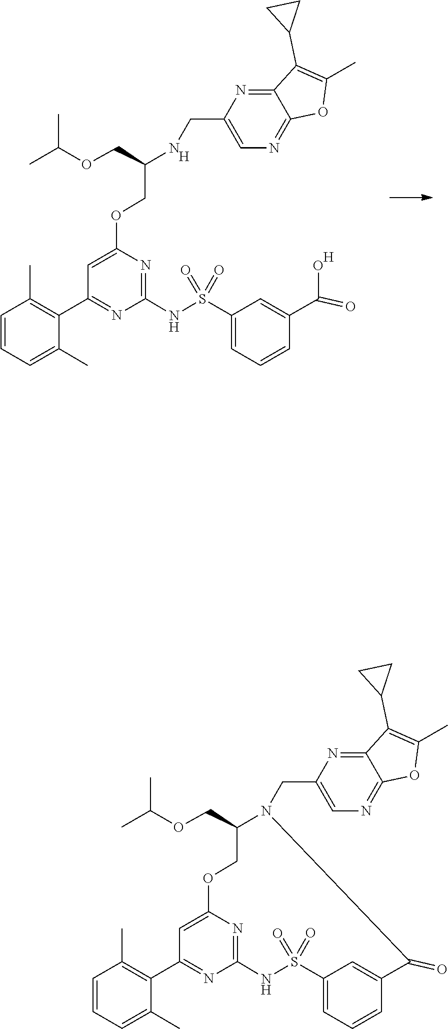

3-[[4-[(2R)-2-[(7-cyclopropyl-6-methyl-furo[2,3-b]pyrazin-2-yl)methylamino]-3-isopropoxy-propoxy]-6-(2,6-dimethylphenyl)pyrimidin-2-yl]sulfamoyl]benzoic acid (hydrochloride salt) (41.1 mg, 0.05240 mmol) was combined with CDMT (17.4 mg, 0.09910 mmol) in DMF (4.1 mL) and cooled to 0° C. N-methylmorpholine (0.02 mL, 0.1819 mmol) was added by syringe and the reaction was stirred at 0° C. for 30 minutes. The ice bath was then removed and stirring was continued for an additional 16 hours at room temperature. The reaction mixture was then partitioned between 50 mL 1 M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-12-[(7-cyclopropyl-6-methyl-furo[2,3-b]pyrazin-2-yl)methyl]-6-(2,6-dimethylphenyl)-11-(isopropoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (22.0 mg, 61%) as a white solid. ¹H NMR (400 MHz, CDCl₃) δ 8.67-8.64 (m, 1H), 8.27 (s, 1H), 7.83-7.79 (m, 1H), 7.56-7.48 (m, 2H), 7.29-7.23 (m, 1H), 7.09 (d, J=7.7 Hz, 2H), 6.24 (s, 1H), 5.38 (dd, J=11.0, 4.3 Hz, 1H), 5.25 (d, J=15.2 Hz, 1H), 4.60 (d, J=15.2 Hz, 1H), 4.44 (t, J=11.4 Hz, 1H), 4.38-4.29 (m, 1H), 3.65-3.56 (m, 2H), 3.44 (hept, J=6.1 Hz, 1H), 2.56 (s, 3H), 2.09 (s, 6H), 2.01 (s, 1H), 1.87-1.78 (m, 1H), 1.33-1.18 (m, 3H), 1.11 (d, J=6.1 Hz, 3H), 1.04-1.02 (m, 3H). ESI-MS m/z calc. 682.2573, found 683.9 (M+1)⁺; Retention time: 2.02 minutes; LC method A.

### Example 49: Preparation of Compound I-30

### Step 1: (2R)-2-Amino-3-tetrahydropyran-4-yl-propan-1-ol

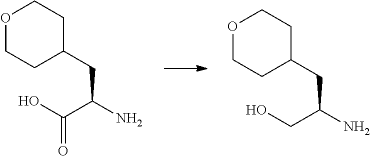 [SMILES: N[C@H](CC1CCOCC1)C(=O)O ; N[C@@H](CO)CC1CCOCC1]

To a solution of (2R)-2-amino-3-tetrahydropyran-4-yl-propanoic acid (hydrochloride salt) (3 g, 14.308 mmol) in THE (30 mL) was added dropwise BH₃·DMS in THE (24 mL of 2 M, 48.000 mmol) at 0° C. and the reaction was stirred for 20 min. The ice bath was removed, and the reaction mixture was allowed to warm up to room temperature and stirred overnight. The reaction was quenched by the addition of MeOH (40 mL). The volatiles were removed, and the crude residue taken up in 1 M HCl (50 mL) and washed with diethyl ether (3×50 mL). The aqueous layer was concentrated and mixed with IPA (10 mL). The mixture was concentrated again and mixed with IPA (10 mL) for 2 more times to give (2R)-2-amino-3-tetrahydropyran-4-yl-propan-1-ol (hydrochloride salt) (2.3 g, 81%) as a white solid. ESI-MS m/z calc. 159.12593, found 159.6 (M+0)⁺; Retention time: 0.76 minutes; LC method E.

### Step 2: 3-[[4-[(2R)-2-Amino-3-tetrahydropyran-4-yl-propoxy]-6-(2,6-dimethylphenyl)pyrimidin-2-yl]sulfamoyl]benzoic acid

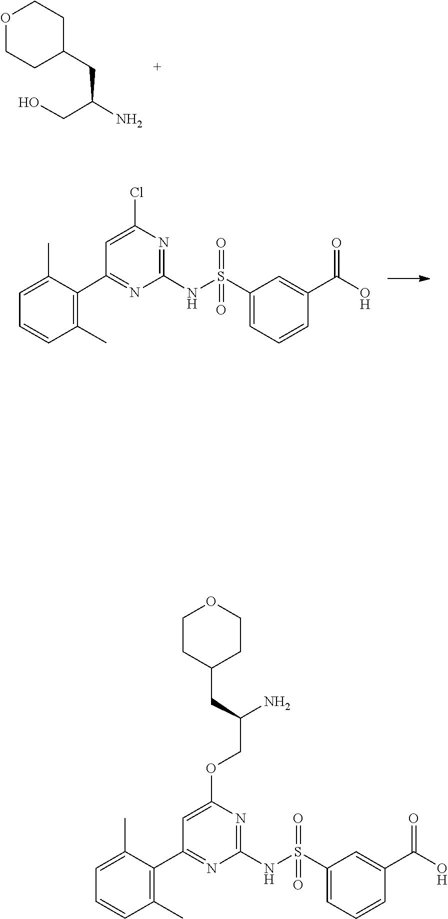 [SMILES: N[C@@H](CO)CC1CCOCC1 ; Cc1cccc(C)c1-c1cc(Cl)nc(NS(=O)(=O)c2cccc(C(=O)O)c2)n1 ; Cc1cccc(C)c1-c1cc(OC[C@H](N)CC2CCOCC2)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (2.6 g, 6.2221 mmol) and (2R)-2-amino-3-tetrahydropyran-4-yl-propan-1-ol (hydrochloride salt) (1.5 g, 7.2821 mmol) in anhydrous THE (30 mL) was added tBuONa (2.7 g, 28.095 mmol) at rt. The reaction was stirred at rt for 1 hour. The reaction was quenched with 1 N HCl (50 mL). The product was extracted with ethyl acetate (3×50 mL). The combined organic layers were washed with brine (50 mL), dried over anhydrous sodium sulfate, and concentrated under vacuum. The residue was triturated with 1:1 ethyl acetate and hexane (20 mL) to furnish 3-[[4-[(2R)-2-amino-3-(cyclobutoxy)propoxy]-6-(2,6-dimethylphenyl)pyrimidin-2-yl]sulfamoyl]benzoic acid (hydrochloride salt) (729 mg, 64%) as an off white solid. ¹H NMR (500 MHz, DMSO) δ 8.45 (s, 1H), 8.19 (s, 1H), 8.12 (dd, J=15.1, 7.8 Hz, 2H), 7.69 (t, J=7.8 Hz, 1H), 7.24 (t, J=7.5 Hz, 1H), 7.11 (d, J=7.5 Hz, 2H), 4.34 (dd, J=11.7, 2.9 Hz, 1H), 4.15 (dd, J=11.5, 6.5 Hz, 1H), 3.86-3.79 (m, 2H), 3.60 (s, 1H), 3.26 (d, J=12.9 Hz, 2H), 1.97 (s, 6H), 1.66-1.57 (m, 3H), 1.57-1.45 (m, 2H), 1.18-1.13 (m, 2H). ESI-MS m/z calc. 540.2043, found 541.3 (M+1)⁺; Retention time: 1.48 minutes; LC method H.

### Step 3: 3-[[4-(2,6-Dimethylphenyl)-6-[(2R)-2-[(6-isopropylfuro[2,3-b]pyrazin-2-yl)methylamino]-3-tetrahydropyran-4-yl-propoxy]pyrimidin-2-yl]sulfamoyl]benzoic acid

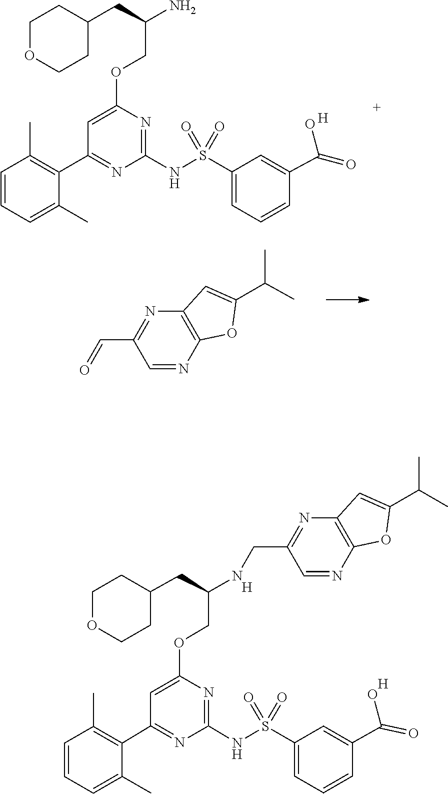

In a 4 mL vial, to a stirred mixture of 3-[[4-[(2R)-2-amino-3-tetrahydropyran-4-yl-propoxy]-6-(2,6-dimethylphenyl)pyrimidin-2-yl]sulfamoyl]benzoic acid (hydrochloride salt) (40 mg, 0.06931 mmol) and 6-isopropylfuro[2,3-b]pyrazine-2-carbaldehyde (13.2 mg, 0.06940 mmol) in anhydrous dichloromethane (300 μL) were added glacial acetic acid (10 μL, 0.1758 mmol) and DIPEA (40 μL, 0.2296 mmol), in that order, at 0° C. under nitrogen. After 2 min, sodium triacetoxyborohydride (52 mg, 0.2454 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish product as a white solid. 3-[[4-(2,6-dimethylphenyl)-6-[(2R)-2-[(6-isopropylfuro[2,3-b]pyrazin-2-yl)methylamino]-3-tetrahydropyran-4-yl-propoxy]pyrimidin-2-yl]sulfamoyl]benzoic acid (hydrochloride salt) (33 mg, 63%). ESI-MS m/z calc. 714.28357, found 715.9 (M+1)⁺; Retention time: 1.34 minutes; LC method A.

### Step 4: (11R)-6-(2,6-Dimethylphenyl)-12-[(6-isopropylfuro[2,3-b]pyrazin-2-yl)methyl]-2,2-dioxo-11-(tetrahydropyran-4-ylmethyl)-9-oxa-2λ⁶-thia-3,5,12,19-tetrazatricyclo[12.3.1.14,8]nonadeca-1(18),4(19),5,7,4,16-hexaen-13-one (Compound I-30

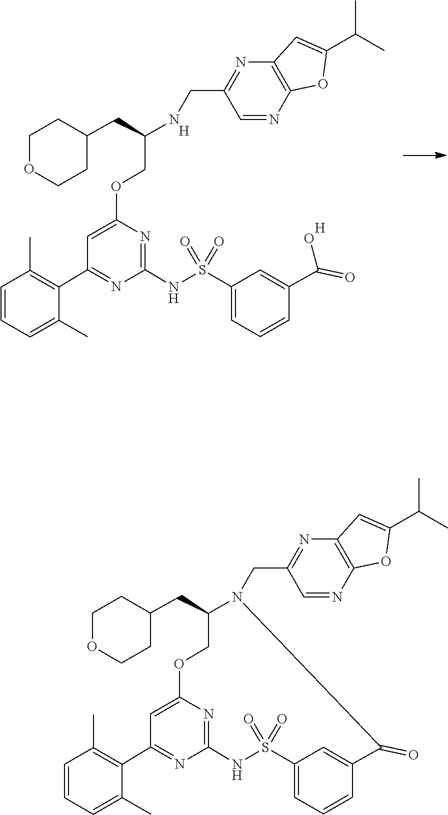

To a stirred solution of 3-[[4-(2,6-dimethylphenyl)-6-[(2R)-2-[(6-isopropylfuro[2,3-b]pyrazin-2-yl)methylamino]-3-tetrahydropyran-4-yl-propoxy]pyrimidin-2-yl]sulfamoyl]benzoic acid (hydrochloride salt) (33 mg, 0.04392 mmol) in anhydrous DMF (1.6 mL) was added 2-chloro-4,6-dimethoxy-1,3,5-triazine (13 mg, 0.07404 mmol) (CDMT), followed by addition of 4-methylmorpholine (35 μL, 0.3183 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished product as a white solid. (11R)-6-(2,6-dimethylphenyl)-12-[(6-isopropylfuro[2,3-b]pyrazin-2-yl)methyl]-2,2-dioxo-11-(tetrahydropyran-4-ylmethyl)-9-oxa-2λ⁶-thia-3,5,12,19-tetrazatricyclo[12.3.1.14,8]nonadeca-1(18),4(19),5,7,14,16-hexaen-13-one (20 mg, 63%) ¹H NMR (400 MHz, CDCl₃) δ 8.69 (s, 1H), 8.42 (s, 1H), 8.16 (d, J=7.8 Hz, 1H), 7.77 (d, J=7.5 Hz, 1H), 7.64 (t, J=7.7 Hz, 1H), 7.19 (t, J=7.5 Hz, 1H), 7.01 (d, J=7.5 Hz, 2H), 6.62 (s, 1H), 6.17 (s, 1H), 5.42 (d, J=10.9 Hz, 1H), 5.33 (d, J=14.9 Hz, 1H), 4.32 (d, J=15.2 Hz, 1H), 4.26 (d, J=11.0 Hz, 1H), 4.23-4.13 (m, 1H), 3.90 (d, J=11.4 Hz, 1H), 3.77 (d, J=11.4 Hz, 1H), 3.33 (t, J=11.7 Hz, 1H), 3.26-3.10 (m, 2H), 1.96 (s, 6H), 1.83 (t, J=12.7 Hz, 1H), 1.55-1.42 (m, 3H), 1.40 (d, J=6.9 Hz, 6H), 1.34-1.22 (m, 1H), 0.97-0.75 (m, 2H). ESI-MS m/z calc. 696.273, found 697.8 (M+1)⁺; Retention time: 1.86 minutes; LC method A.

### Example 50: Preparation of Compound I-36 and Compound I-37

### Step 1: 3-[[4-(2,6-Dimethylphenyl)-6-[5-fluoro-2-[(6-isopropylfuro[2,3-b]pyrazin-2-yl)methylamino]-5-methyl-hexoxy]pyrimidin-2-yl]sulfamoyl]benzoic acid

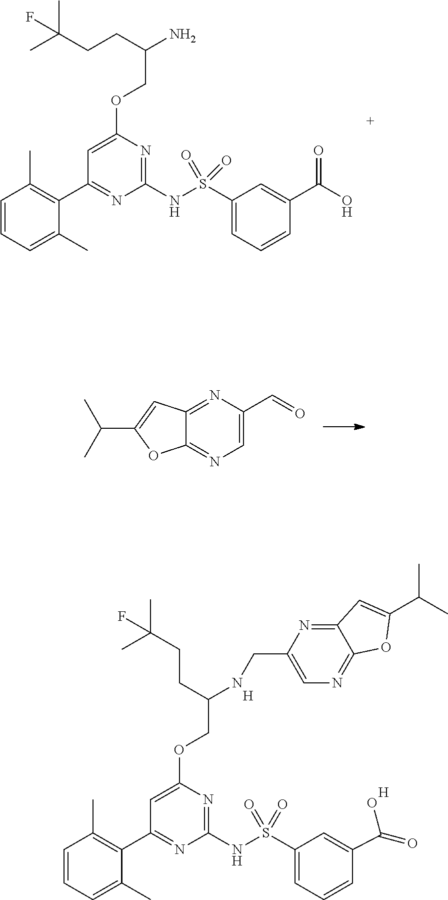

A 4 mL vial was charged under nitrogen with 3-[[4-(2-amino-5-fluoro-5-methyl-hexoxy)-6-(2,6-dimethylphenyl)pyrimidin-2-yl]sulfamoyl]benzoic acid (hydrochloride salt) (84.8 mg, 0.1388 mmol), 6-isopropylfuro[2,3-b]pyrazine-2-carbaldehyde (27.6 mg, 0.1444 mmol), anhydrous DCM (1000 μL), acetic acid (20 μL, 0.3517 mmol) and DIEA (90 μL, 0.5167 mmol). The mixture was cooled down in an ice bath, sodium triacetoxyborohydride (105 mg, 0.4954 mmol) was added and the reaction was stirred for 1.5 h. The reaction was quenched with aqueous 1 M HCl (1 mL), MeOH (0.5 mL) and DMSO (0.5 mL), filtered and purified ion by reverse phase HPLC (1-50% acetonitrile/5 mM aqueous HCl over 30 min) to give as a white solid 3-[[4-(2,6-dimethylphenyl)-6-[5-fluoro-2-[(6-isopropylfuro[2,3-b]pyrazin-2-yl)methylamino]-5-methyl-hexoxy]pyrimidin-2-yl]sulfamoyl]benzoic acid (hydrochloride salt) (52.0 mg, 51%). ESI-MS m/z calc. 704.27924, found 705.2 (M+1)⁺; Retention time: 1.48 minutes; LC method A.

### Step 2: 6-(2,6-Dimethylphenyl)-11-(3-fluoro-3-methyl-butyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one

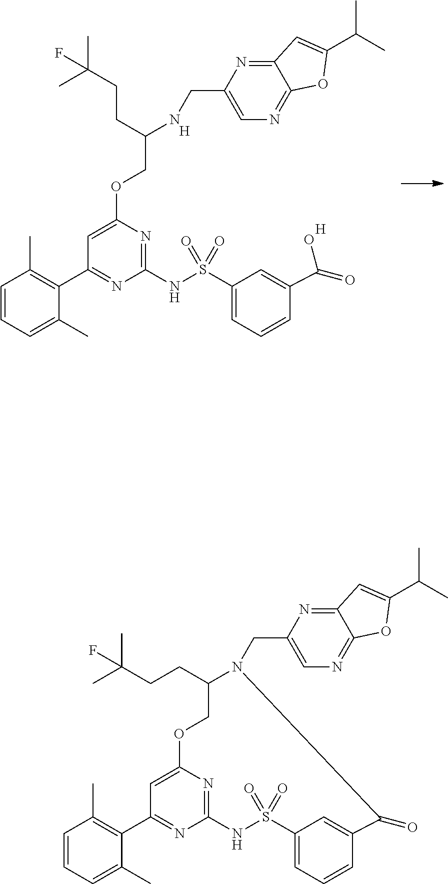

3-[[4-(2,6-dimethylphenyl)-6-[5-fluoro-2-[(6-isopropylfuro[2,3-b]pyrazin-2-yl)methylamino]-5-methyl-hexoxy]pyrimidin-2-yl]sulfamoyl]benzoic acid (hydrochloride salt) (52.0 mg, 0.07015 mmol) was combined under nitrogen with CDMT (33 mg, 0.1880 mmol) and DMF (2.0 mL). The solution was stirred at 0° C. 4-Methyl-morpholine (52 μL, 0.4730 mmol) was added and the mixture was stirred in the cooling bath that was allowed to warm to room temperature. After 16 h, the reaction was filtered and purified by reverse phase HPLC (1-99% acetonitrile/5 mM aqueous HCl over 30 min) to give as a white solid 6-(2,6-dimethylphenyl)-11-(3-fluoro-3-methyl-butyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (31.3 mg, 65%), ESI-MS m/z calc. 686.2687, found 687.2 (M+1)⁺; Retention time: 2.09 minutes; LC method A.

### Step 3: (11R)-6-(2,6-Dimethylphenyl)-11-(3-fluoro-3-methyl-butyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one, SFC peak 1, Compound I-36, and (11S)-6-(2,6-dimethylphenyl)-11-(3-fluoro-3-methyl-butyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one, SFC peak 2, Compound I-37

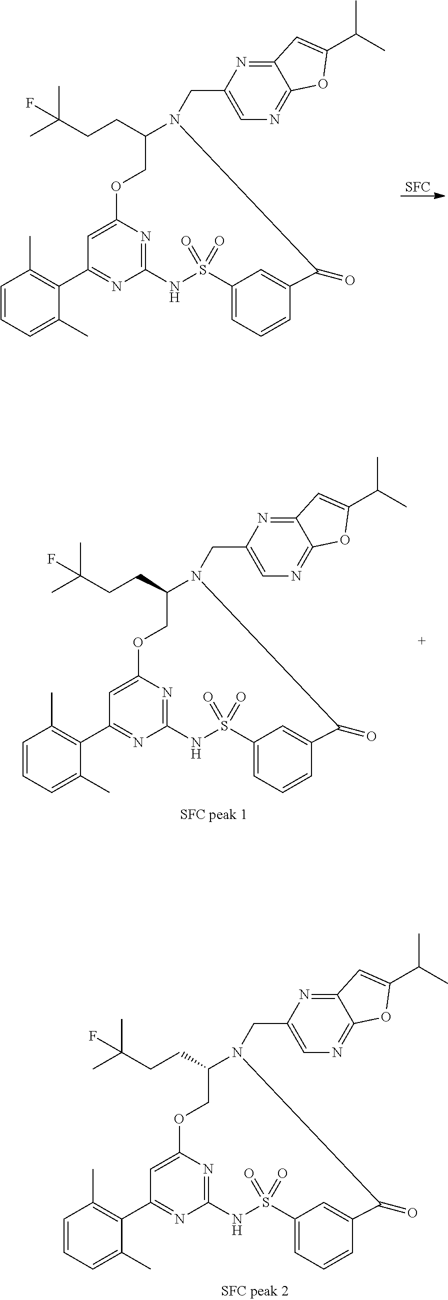 [SMILES: Cc1cccc(C)c1-c1cc2nc(n1)NS(=O)(=O)c1cccc(c1)C(=O)N(Cc1cnc3oc(C(C)C)cc3n1)C(CCC(C)(C)F)CO2 ; "SFC" ; Cc1cccc(C)c1-c1cc2nc(n1)NS(=O)(=O)c1cccc(c1)C(=O)N(Cc1cnc3oc(C(C)C)cc3n1)[C@H](CCC(C)(C)F)CO2 ; Cc1cccc(C)c1-c1cc2nc(n1)NS(=O)(=O)c1cccc(c1)C(=O)N(Cc1cnc3oc(C(C)C)cc3n1)[C@@H](CCC(C)(C)F)CO2]

6-(2,6-Dimethylphenyl)-11-(3-fluoro-3-methyl-butyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one racemic (31.3 mg, 0.04557 mmol) was subjected to SFC separation (ChiralCel OD (21.2×250 mm, 5 μM) column at 40° C., mobile phase of 27% MeOH (+20 mM NH₃), isocratic mode, flow of 70 mL/min, sample concentration of 30 mg/mL in methanol, injection volume of 325 μL, pressure of 197 bar). The collected fractions afforded two peaks by chiral column analysis. The first peak to elute on the OD column produced the material which was then concentrated and purified by reverse phase HPLC (1-99% acetonitrile/5 mM aqueous HCl over 15 min) to afford as a white solid (SFC peak 1) (11R)-6-(2,6-dimethylphenyl)-11-(3-fluoro-3-methyl-butyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (6.0 mg, 38%). ¹H NMR (400 MHz, Chloroform-d) δ 10.11 (s, 1H), 8.63 (t, J=1.8 Hz, 1H), 8.42 (s, 1H), 7.99 (d, J=7.9 Hz, 1H), 7.80 (dt, J=7.7, 1.4 Hz, 1H), 7.64 (t, J=7.8 Hz, 1H), 7.21 (t, J=7.6 Hz, 1H), 7.03 (d, J=7.6 Hz, 2H), 6.62 (d, J=1.0 Hz, 1H), 6.19 (s, 1H), 5.43-5.28 (m, 2H), 4.36-4.22 (m, 2H), 4.14-4.04 (m, 1H), 3.22-3.09 (m, 1H), 1.99 (s, 6H), 1.91-1.79 (m, 2H), 1.60-1.50 (m, 2H), 1.40 (d, J=6.9 Hz, 6H), 1.29 (dd, J=21.3, 4.1 Hz, 6H). ESI-MS m/z calc. 686.2687, found 687.2 (M+1)⁺; Retention time: 2.06 minutes. The second peak to elute on the OD column produced the material which was then concentrated and purified by reverse phase HPLC (1-99% acetonitrile/5 mM aqueous HCl over 15 min) to afford as a white solid (SFC peak 2) (11S)-6-(2,6-dimethylphenyl)-11-(3-fluoro-3-methyl-butyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (5.7 mg, 36%). ¹H NMR (400 MHz, Chloroform-d) δ 10.30 (s, 1H), 8.63 (t, J=1.8 Hz, 1H), 8.42 (s, 1H), 7.99 (d, J=7.9 Hz, 1H), 7.80 (dt, J=7.7, 1.3 Hz, 1H), 7.63 (t, J=7.8 Hz, 1H), 7.21 (t, J=7.6 Hz, 1H), 7.02 (d, J=7.6 Hz, 2H), 6.62 (d, J=1.0 Hz, 1H), 6.18 (s, 1H), 5.42-5.31 (m, 2H), 4.35-4.22 (m, 2H), 4.13-4.02 (m, 1H), 3.24-3.09 (m, 1H), 1.98 (s, 6H), 1.91-1.79 (m, 2H), 1.62-1.53 (m, 2H), 1.40 (d, J=6.9 Hz, 6H), 1.32 (d, J=4.3 Hz, 3H), 1.27 (d, J=4.4 Hz, 3H). ESI-MS m/z calc. 686.2687, found 687.2 (M+1)⁺; Retention time: 2.09 minutes. LC method A.

### Example 51: Preparation of Compound I-49 and Compound I-50

### Step 1: Methyl (2R)-2-(benzyloxycarbonylamino)-3-(cyclobutoxy)propanoate

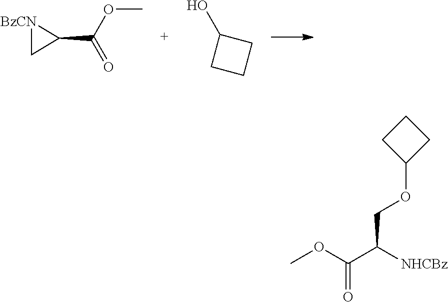

To a solution of O1-benzyl O2-methyl (2R)-aziridine-1,2-dicarboxylate (2 g, 8.5021 mmol) and cyclobutanol (2.2925 g, 2.5 mL, 31.794 mmol) in anhydrous DCM (20 mL) was added boron trifluoride diethyl etherate (115.00 mg, 0.1 mL, 0.8103 mmol) at 0° C. The reaction was stirred at rt overnight. The reaction mixture was diluted with water (50 mL) and DCM (50 mL). Two layers were separated, and the aqueous layer was extracted with DCM (2×50 mL). The combined organic layers were dried over anhydrous sodium sulfate and concentrated under vacuum. The residue was purified by silica gel chromatography using 0 to 40% ethyl acetate in hexane to furnish methyl (2R)-2-(benzyloxycarbonylamino)-3-(cyclobutoxy)propanoate (1.83 g, 63%) as white solid. ¹H NMR (400 MHz, CDCl₃) δ 7.44-7.30 (m, 5H), 5.63 (d, J=8.5 Hz, 1H), 5.14 (s, 2H), 4.48 (dt, J=8.6, 3.1 Hz, 1H), 3.90 (m, 1H), 3.78 (s, 1H), 3.57 (dd, J=9.0, 3.3 Hz, 1H), 2.21-2.09 (m, 2H), 1.95-1.78 (m, 2H), 1.73-1.61 (m, 1H), 1.56-1.40 (m, 1H). ESI-MS m/z calc. 307.14197, found 308.5 (M+1)⁺; Retention time: 2.98 minutes; LC method E.

### Step 2: Benzyl N-[(1S)-1-(cyclobutoxymethyl)-2-hydroxy-ethyl]carbamate

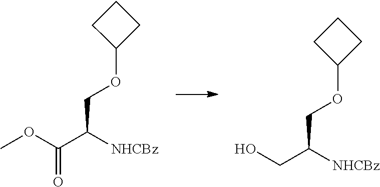

To a solution of methyl (2R)-2-(benzyloxycarbonylamino)-3-(cyclobutoxy)propanoate (1.83 g, 5.9543 mmol) in a solvent mixture of THF (18 mL) and methanol (7 mL) was added sodium borohydride (592 mg, 15.648 mmol). The reaction was stirred at rt for 3 hours. The residue was diluted with ethyl acetate (100 mL) and washed with brine (30 mL), dried over anhydrous sodium sulfate, and concentrated under vacuum. The residue was purified by silica gel chromatography using 0 to 30% acetone in hexane to furnish benzyl N-[(1S)-1-(cyclobutoxymethyl)-2-hydroxy-ethyl]carbamate (1.34 g, 69%) as a white solid. ¹H NMR (400 MHz, Chloroform) δ 7.41-7.32 (m, 5H), 5.13 (s, 2H), 3.97-3.64 (m, 4H), 3.62-3.44 (m, 2H), 1.87 (m, 2H), 1.70 (m, 1H), 1.55-1.43 (m, 3H). ESI-MS m/z calc. 279.14706, found 280.3 (M+1)⁺; Retention time: 2.41 minutes; LC method E.

### Step 3: (2S)-2-Amino-3-(cyclobutoxy)propan-1-ol

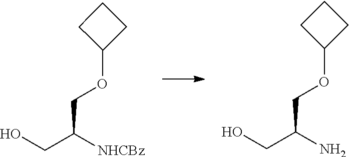

To a solution of benzyl N-[(1S)-1-(cyclobutoxymethyl)-2-hydroxy-ethyl]carbamate (100 mg, 0.3769 mmol) in ethyl acetate (2 mL) was added 10% Pd/C (41 mg, 10% w/w, 0.0385 mmol). The reaction was hydrogenated under 1 atm of hydrogen gas for 3 hours. The catalyst was removed by filtration through a pad of Celite. The filtrate was concentrated under vacuum to furnish (2S)-2-amino-3-(cyclobutoxy)propan-1-ol (80 mg, 73%) as a clear gel. ¹H NMR (400 MHz, CDCl₃) δ 3.98-3.88 (m, 1H), 3.63 (dd, J=10.8, 4.6 Hz, 1H), 3.52 (dd, J=10.8, 5.7 Hz, 1H), 3.35 (ddd, J=15.6, 9.4, 5.5 Hz, 2H), 3.11-3.01 (m, 1H), 2.26-2.13 (m, 2H), 1.97-1.83 (m, 2H), 1.59-1.35 (m, 1H), 1.35-1.30 (m, 1H). ESI-MS m/z calc. 145.11028, found 145.1 (M+0)⁺; Retention time: 0.95 minutes; LC method E.

### Step 4: 3-[[4-[(2R)-2-Amino-3-(cyclobutoxy)propoxy]-6-(2,6-dimethylphenyl)pyrimidin-2-yl]sulfamoyl]benzoic acid

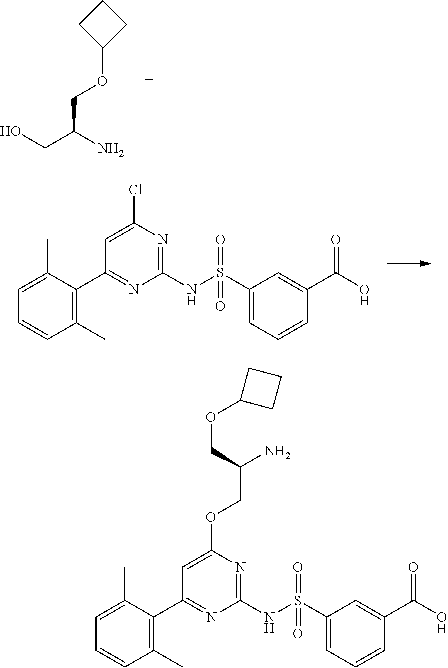

To a solution of 3-[[4-chloro-6-(2,6-dimethylphenyl)pyrimidin-2-yl]sulfamoyl]benzoic acid (1.06 g, 2.5367 mmol) and (2S)-2-amino-3-(cyclobutoxy)propan-1-ol (300 mg, 1.9628 mmol) in anhydrous THE (7.5 mL) was added tBuONa (1.4 g, 14.568 mmol) at rt. The reaction was stirred at rt for 1 hour. The reaction was quenched with 1 N HCl (30 mL). The product was extracted with ethyl acetate (3×50 mL). The combined organic layers were washed with brine (30 mL), dried over anhydrous sodium sulfate, and concentrated under vacuum. The residue was triturated with 1:1 ethyl acetate and hexane (20 mL) and residue was purified by reverse phase HPLC (Buffer A: water buffered with 5 mM HCl;, Buffer B:100% ACN, eluting from 20 to 65% over a 40 min gradient) to furnish 3-[[4-[(2R)-2-amino-3-(cyclobutoxy)propoxy]-6-(2,6-dimethylphenyl)pyrimidin-2-yl]sulfamoyl]benzoic acid (hydrochloride salt) (729 mg, 64%) as an off white solid containing about 20% of the opposite enantiomer. ¹H NMR (500 MHz, DMSO) δ 8.43 (s, 1H), 8.12 (t, J=7.4 Hz, 2H), 7.67 (t, J=7.8 Hz, 1H), 7.37-7.16 (m, 1H), 7.11 (d, J=7.6 Hz, 2H), 6.29 (s, 1H), 4.49-4.20 (m, 2H), 4.03-3.88 (m, 1H), 3.76-3.60 (m, 1H), 3.57-3.49 (m, 1H), 3.48-3.38 (m, 1H), 2.20-2.07 (m, 2H), 1.98 (s, 6H), 1.91-1.78 (m, 2H), 1.67-1.57 (m, 1H), 1.57-1.35 (m, 1H). ESI-MS m/z calc. 526.1886, found 527.2 (M+1)⁺; Retention time: 1.65 minutes; LC method H.

### Step 5: 3-[[4-[3-(Cyclobutoxy)-2-[(6-isopropylfuro[2,3-b]pyrazin-2-yl)methylamino]propoxy]-6-(2,6-dimethylphenyl)pyrimidin-2-yl]sulfamoyl]benzoic acid

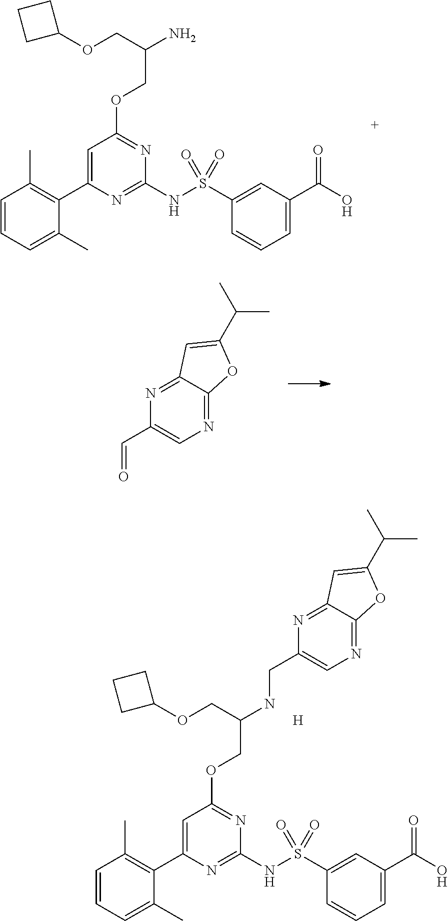

In a 4 mL vial, 3-[[4-[2-amino-3-(cyclobutoxy)propoxy]-6-(2,6-dimethylphenyl)pyrimidin-2-yl]sulfamoyl]benzoic acid (hydrochloride salt) (90 mg, 0.1598 mmol, ˜80:20 mixture of enantiomers) and 6-isopropylfuro[2,3-b]pyrazine-2-carbaldehyde (33 mg, 0.1735 mmol) were combined in DCM (1 mL). Glacial acetic acid (14 μL, 0.2462 mmol) was added. The mixture was cooled to 0° C. before the addition of DIEA (70 μL, 0.4019 mmol). After stirring for 15 minutes, sodium triacetoxyborohydride (102 mg, 0.4813 mmol) was added. Stirring was continued at 0° C. for 30 minutes. The reaction was quenched with the addition of methanol and aqueous 1 M HCl. After filtration, purification by reverse phase HPLC (10-99% acetonitrile/5 mM aqueous HCl over 15 minutes) gave 3-[[4-[3-(cyclobutoxy)-2-[(6-isopropylfuro[2,3-b]pyrazin-2-yl)methylamino]propoxy]-6-(2,6-dimethylphenyl)pyrimidin-2-yl]sulfamoyl]benzoic acid (hydrochloride salt) (87 mg, 74%) as a slightly beige solid. ESI-MS m/z calc. 700.26794, found 701.5 (M+1)⁺; Retention time: 1.18 minutes; LC method A.

### Step 6: 11-(Cyclobutoxymethyl)-6-(2,6-dimethylphenyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, SFC peak 1, Compound I-49, and 11-(cyclobutoxymethyl)-6-(2,6-dimethylphenyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one, SFC peak 2, Compound I-50

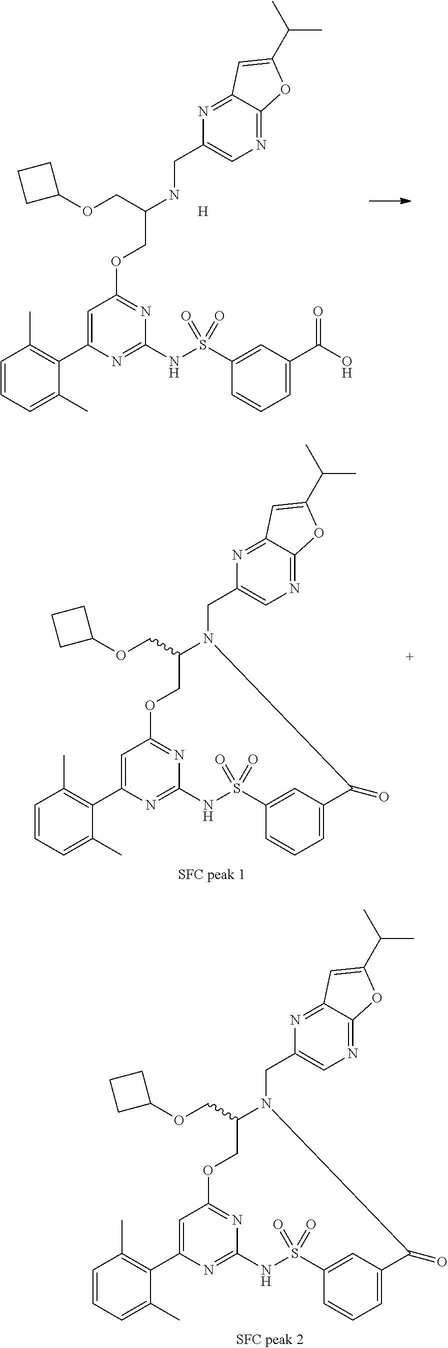

In a 4 mL vial, 3-[[4-[3-(cyclobutoxy)-2-[(6-isopropylfuro[2,3-b]pyrazin-2-yl)methylamino]propoxy]-6-(2,6-dimethylphenyl)pyrimidin-2-yl]sulfamoyl]benzoic acid (hydrochloride salt) (87 mg, 0.1180 mmol) and 2-chloro-4,6-dimethoxy-1,3,5-triazine (31 mg, 0.1766 mmol) were combined in DMF (900 μL). The solution was cooled before the addition of 4-methylmorpholine (65 μL, 0.5912 mmol). The reaction mixture was allowed to slowly warm to room temperature and stirred overnight. Purification by reverse phase HPLC (10-99% acetonitrile/5 mM aqueous HCl over 15 minutes) gave a slightly yellow solid (65 mg). The enantiomers were separated by chiral SFC using a ChiralCel OD column (21.2×250 mm, 5 μm) at 50° C. Mobile phase was 20% MeOH (20 mM NH₃), 80% CO₂ at a 70 mL/min flow. Concentration of the sample was 26 mg/mL in methanol, injection volume 500 μL with an outlet pressure of 154 bar, detection wavelength of 210 nm. This provided 11-(cyclobutoxymethyl)-6-(2,6-dimethylphenyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (32.0 mg, 78%) as Peak 1, ¹H NMR (400 MHz, Chloroform-d) δ 10.09 (s, 1H), 8.64 (s, 1H), 8.38 (s, 1H), 7.82 (d, J=7.6, 1.4 Hz, 1H), 7.64 (d, J=7.8 Hz, 1H), 7.55 (t, J=7.7 Hz, 1H), 7.23 (d, J=7.6 Hz, 1H), 7.07 (d, J=7.6 Hz, 2H), 6.62 (s, 1H), 6.21 (s, 1H), 5.42-5.30 (m, 2H), 4.57 (d, J=15.1 Hz, 1H), 4.46 (t, J=11.6 Hz, 1H), 4.38-4.29 (m, 1H), 3.78 (p, 1H), 3.61-3.53 (m, 1H), 3.48 (dd, J=10.7, 3.1 Hz, 1H), 3.16 (hept, J=6.9, 1.1 Hz, 1H), 2.21-2.06 (m, 2H), 2.04 (s, 6H), 1.89-1.75 (m, 2H), 1.71-1.63 (m, 1H), 1.52-1.43 (m, 1H), 1.41 (s, 3H), 1.39 (s, 3H). ESI-MS m/z calc. 682.2573, found 683.6 (M+1)⁺; Retention time: 1.66 minutes, and provided 11-(cyclobutoxymethyl)-6-(2,6-dimethylphenyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (4.8 mg, 12%) as Peak 2, ESI-MS m/z calc. 682.2573, found 683.6 (M+1)⁺; Retention time: 1.65 minutes. LC method A.

### Example 52: Preparation of Compound I-52

### Step 1: Tricyclo[1.1.1]pentane

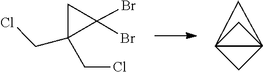 [SMILES: ClCC1(CCl)CC1(Br)Br ; C1C23CC12C3]

MeLi (solution in diethyl ether) (48 mL of 1.6 M, 76.800 mmol) was slowly added to a solution of 1,1-dibromo-2,2-bis(chloromethyl)cyclopropane (10 g, 33.691 mmol) in diethyl ether (25 mL) during 1 h while maintaining the internal reaction temperature between −50 and −60° C. The reaction mixture was then warmed-up to 0° C. and stirred at this temperature for 30 min. The reaction flask was attached to a bent tube equipped with vacuum adapter and a receiving flask. The system was placed under vacuum using a water vacuum aspirator and the reaction mixture was distilled into the receiving flask that was cooled with liquid nitrogen while maintaining the distillation flask at around 0° C. with a water-ice bath to provide a distillate solution containing about 3.5% of tricyclo[1.1.1.01,3]pentane (53 g, 83%) as a clear solution ¹H NMR (400 MHz, CDCl₃) δ 1.96 (s, 6H). in diethyl ether. This solution was used directly in the next reaction without further purification.

### Step 2: Methyl (2R)-2-(tert-butoxycarbonylamino)-3-(3-iodo-1-bicyclo[1.1.1]pentanyl)propanoate

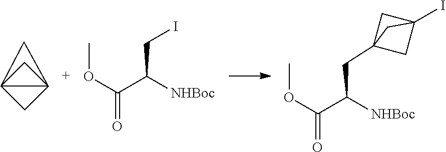

Methyl (2S)-2-(tert-butoxycarbonylamino)-3-iodo-propanoate (3 g, 9.1149 mmol) was dissolved in a solution of tricyclo[1.1.1.01,3]pentane (in DCM) (53 g, 28.063 mmol) from the previous experiment and then triethylborane (in hexanes) (1.1 mL of 1 M, 1.1000 mmol) was added. This reaction mixture was stirred 1 h at room temperature. The reaction mixture was concentrated under reduced pressure and the resulting residue was purified by reverse phase chromatography on 50 g C₁₈ RediSep Rf gold column using a 5 to 100% gradient of acetonitrile in pure water to provide methyl (2R)-2-(tert-butoxycarbonylamino)-3-(3-iodo-1-bicyclo[1.1.1]pentanyl)propanoate (1.64 g, 45%) clear oil that crystallized on standing. ¹H NMR (400 MHz, CDCl₃) δ 4.98 (d, J=7.3 Hz, 1H), 4.37-4.23 (m, 1H), 3.74 (s, 3H), 2.31-2.21 (m, 6H), 2.19-2.08 (m, 1H), 1.88 (dd, J=14.7, 7.6 Hz, 1H), 1.45 (s, 9H) ESI-MS m/z calc. 395.0594, found 296.0 (M−99)⁺; Retention time: 1.95 minutes, along with a sample of lower purity methyl (2R)-2-(tert-butoxycarbonylamino)-3-(3-iodo-1-bicyclo[1.1.1]pentanyl) propanoate (1.63 g, 35%) yellow oil that crystallized on standing ¹H NMR (400 MHz, CDCl₃) δ 4.98 (d, J=7.8 Hz, 1H), 4.36-4.25 (m, 1H), 3.74 (s, 3H), 2.31-2.19 (m, 6H), 2.16-2.10 (m, 1H), 1.88 (dd, J=14.4, 7.6 Hz, 1H), 1.45 (s, 9H). ESI-MS m/z calc. 395.0594, found 296.0 (M−99)⁺; Retention time: 1.96 minutes; LC method I.

### Step 3: Methyl (2R)-3-(1-bicyclo[1.1.1]pentanyl)-2-(tert-butoxycarbonylamino)propanoate

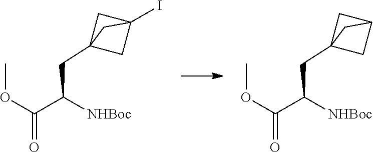 [SMILES: COC(=O)[C@@H](CC12CC(I)(C1)C2)NC(=O)OC(C)(C)C ; COC(=O)[C@@H](CC12CC(C1)C2)NC(=O)OC(C)(C)C]

Triethylborane (in hexanes) (0.5 mL of 1 M, 0.5000 mmol) was added dropwise to a solution of methyl (2R)-2-(tert-butoxycarbonylamino)-3-(3-iodo-1-bicyclo[1.1.1]pentanyl)propanoate (1.64 g, 4.1287 mmol), 2,6-lutidine (1.288 g, 1.4 mL, 12.020 mmol) and tris(trimethylsilyl)silane (3.0628 g, 3.8 mL, 12.317 mmol) in anhydrous THE (9 mL) and left stirring at room temperature over week-end. The reaction mixture was concentrated under reduced pressure and the resulting residue was purified on silica gel with a 40 g column using a 0 to 50% ethyl acetate in heptanes gradient to provide methyl (2R)-3-(1-bicyclo[1.1.1]pentanyl)-2-(tert-butoxycarbonylamino)propanoate (766 mg, 65%) as a pale yellow powder. ¹H NMR (400 MHz, CDCl₃) δ 4.94 (d, J=7.3 Hz, 1H), 4.35-4.25 (m, 1H), 3.73 (s, 3H), 2.45 (s, 1H), 2.04-1.91 (m, 1H), 1.83-1.69 (m, 7H), 1.45 (s, 9H). ESI-MS m/z calc. 269.16272, found 170.2 (M−99)⁺; Retention time: 1.91 minutes; LC method L.

### Step 4: tert-Butyl N-[(1R)-1-(1-bicyclo[1.1.1]pentanylmethyl)-2-hydroxy-ethyl]carbamate

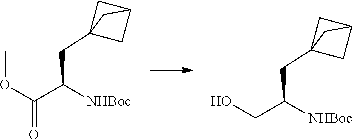 [SMILES: COC(=O)[C@@H](CC12CC(C1)C2)NC(=O)OC(C)(C)C ; CC(C)(C)OC(=O)N[C@@H](CO)CC12CC(C1)C2]

LiBH₄ (350 mg, 16.067 mmol) was added to a solution of methyl (2R)-3-(1-bicyclo[1.1.1]pentanyl)-2-(tert-butoxycarbonylamino)propanoate (1.46 g, 5.1497 mmol) in THE (15 mL) maintained at 0° C. with an ice water bath. The reaction mixture was maintained at this temperature for 30 min and then left to warm up and stirred at room temperature for 2 h. The reaction was then cooled down to 0° C. and quenched by the addition of an aqueous saturated solution of ammonium chloride (15 mL). The biphasic mixture was stirred for 20 minutes at 0° C. then the layers were separated, and the aqueous layer was extracted with ethyl acetate (4×25 mL). The combined organic layers were washed with brine (150 mL), dried over magnesium sulfate, filtered, and concentrated under reduced pressure to afford crude and partially quenched material. A sample of this material tert-butyl N-[(1R)-1-(1-bicyclo[1.1.1]pentanylmethyl)-2-hydroxy-ethyl]carbamate (100 mg, 7%) (white powder) was kept and set aside. ESI-MS m/z calc. 241.1678, found 264.2 (M+23)⁺; Retention time: 1.73 minutes. The rest of the crude material was dissolved back in DCM (50 mL) and washed with 0.2 M aqueous HCl (50 mL) until hydrogen gas stopped evolving. The aqueous phase was separated, washed with DCM (50 mL). The organic phases were combined, dried with magnesium sulfate, filtered and concentrated under reduced pressure to provide tert-butyl N-[(1R)-1-(1-bicyclo[1.1.1]pentanyl methyl)-2-hydroxy-ethyl]carbamate (1.15 g, 88%) as a white solid. ¹H NMR (400 MHz, CDCl₃) δ 4.55 (br. s., 1H), 3.75-3.58 (m, 2H), 3.56-3.47 (m, 1H), 2.59-2.34 (m, 2H), 1.78-1.70 (m, 6H), 1.65 (dd, J=14.7, 4.9 Hz, 1H), 1.53 (dd, J=14.7, 8.6 Hz, 1H), 1.46 (s, 9H). ESI-MS m/z calc. 241.1678, found 264.2 (M+23)⁺; null (M−)⁺; Retention time: 1.73 minutes; LC method J.

### Step 5: (2R)-2-Amino-3-(1-bicyclo[1.1.1]pentanyl)propan-1-ol

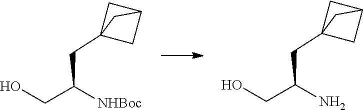 [SMILES: CC(C)(C)OC(=O)N[C@@H](CO)CC12CC(C1)C2 ; N[C@@H](CO)CC12CC(C1)C2]

tert-Butyl N-[(1R)-1-(1-bicyclo[1.1.1]pentanylmethyl)-2-hydroxy-ethyl]carbamate (1.15 g, 4.4794 mmol) was added in small portions to a solution of HCl (in dioxane) (40 mL of 4 M, 160.00 mmol) stirring at room temperature then left at this temperature for 1 h. The reaction mixture was concentrated under reduced pressure. The resulting residue was triturated in anhydrous THE (20 mL), filtered, and washed with more THE (2×5 mL). The resulting solid was dried under high vacuum for 1 h to provide (2R)-2-amino-3-(1-bicyclo[1.1.1]pentanyl) propan-1-ol (hydrochloride salt) (730 mg, 87%) as a white powder ¹H NMR (400 MHz, DMSO-d6) δ 7.91 (br. s., 3H), 5.31 (t, J=5.1 Hz, 1H), 3.62 (dt, J=11.7, 3.7 Hz, 1H), 3.39 (dt, J=11.7, 6.0 Hz, 1H), 3.07-2.96 (m, 1H), 2.45 (s, 1H), 1.78-1.61 (m, 8H). ESI-MS m/z calc. 141.11537, found 142.2 (M+1)⁺; Retention time: 0.57 minutes; LC method I.

### Step 6: (2R)-3-(1-Bicyclo[1.1.1]pentanyl)-2-[(6-cyclopropylfuro[2,3-b]pyrazin-2-yl)methylamino]propan-1-ol

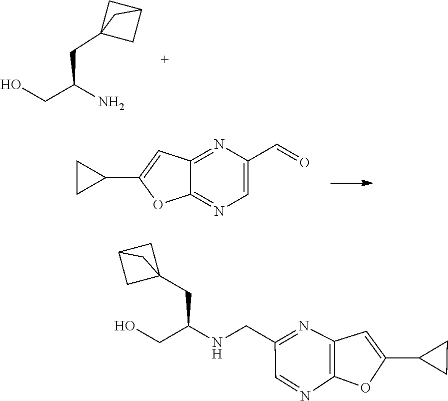

DIEA (200 μL, 1.148 mmol) was added to a suspension of (2R)-2-amino-3-(1-bicyclo[1.1.1]pentanyl)propan-1-ol (hydrochloride salt) (182 mg, 1.024 mmol) and 6-cyclopropylfuro[2,3-b]pyrazine-2-carbaldehyde (193 mg, 1.026 mmol) in DCE (2.7 mL) and the mixture was stirred for 10 minutes until all solids dissolved. Acetic acid (70 μL, 1.231 mmol) was added. After stirring for 2 hours at room temperature, the mixture was cooled down in an ice-bath and sodium triacetoxyborohydride (400 mg, 1.887 mmol) was added in 3 equal portions. After 5 min, the cooling bath was removed, and the reaction was stirred at room temperature for 3 hours. Aqueous HCl (3.4 mL of 1 M, 3.400 mmol) was added dropwise, and the mixture was stirred for 10 minutes at room temperature before being cooled down in an ice-water bath. Aqueous NaOH (750 mg of 50% w/w, 9.376 mmol) was slowly added dropwise. The mixture was poured into a separatory funnel and the layers were separated. The aqueous layer was extracted with DCM (20 mL). The combined organic extracts were washed with water (30 mL), brine (30 mL), dried over sodium sulfate and concentrated to give an orange oil. The material was dissolved in DCM (50 mL), filtered over a pad of celite. The material was triturated in 8 mL of hot (90° C.) heptane for 10 minutes. The suspension was allowed to cool to room temperature and was further triturated and stirred for 30 min. The solid was collected by filtration and washed with a small amount of heptane. Drying under vacuum provided (2R)-3-(1-bicyclo[1.1.1]pentanyl)-2-[(6-cyclopropylfuro[2,3-b]pyrazin-2-yl)methylamino]propan-1-ol (215 mg, 64%). ¹H NMR (400 MHz, Chloroform-d) δ 8.08 (s, 1H), 6.58 (s, 1H), 4.12-3.94 (m, 2H), 3.69 (dd, J=10.9, 3.7 Hz, 1H), 3.38 (dd, J=11.0, 6.0 Hz, 1H), 2.84-2.72 (m, 1H), 2.47 (s, 1H), 2.15-2.10 (m, 1H), 2.01 (s, 2H), 1.72 (s, 6H), 1.68 (dd, J=6.1 Hz, 1H), 1.59 (dd, J=14.6, 6.7 Hz, 1H), 1.19-1.11 (m, 4H). ESI-MS m/z calc. 313.17902, found 314.6 (M+1)⁺; Retention time: 0.42 minutes; LC method B.

### Step 7: 3-[[4-[(2R)-3-(1-Bicyclo[1.1.1]pentanyl)-2-[(6-cyclopropylfuro[2,3-b]pyrazin-2-yl)methylamino]propoxy]-6-(2,6-dimethylphenyl)pyrimidin-2-yl]sulfamoyl]benzoic acid

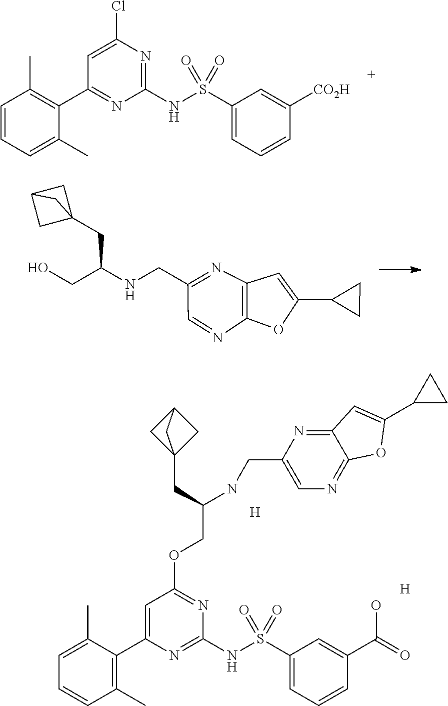

(2R)-3-(1-bicyclo[1.1.1]pentanyl)-2-[(6-cyclopropylfuro[2,3-b]pyrazin-2-yl)methylamino]propan-1-ol (212 mg, 0.6765 mmol), 3-[[4-chloro-6-(2,6-dimethylphenyl)pyrimidin-2-yl]sulfamoyl]benzoic acid (287 mg, 0.6868 mmol) were combined in anhydrous THE (2 mL) in a nitrogen purged vial. The mixture was stirred at room temperature it became homogeneous (10 minutes). Sodium tert-butoxide (335 mg, 3.486 mmol) was added in a single portion, and the reaction became warm to the touch. Stirring was continued with no external heating for 1 hour. The reaction mixture was cooled in an ice-water bath and then quenched by dropwise addition of an aqueous HCl (8 mL of 1 M, 8.000 mmol) solution. Brine (5 mL) and ethyl acetate (15 mL) were added. The layers were separated and the aqueous was extracted an additional 2× ethyl acetate (10 mL). The combined organics were washed with brine and dried over sodium sulfate. The solution was concentrated under vacuum (water bath at 50° C.) until solids started to form. The resulting slurry was diluted slightly with ethyl acetate, and cooled to 0° C. in an ice bath. After stirring for 40 minutes the solids were collected by filtration and washed with cold ethyl acetate, to give after drying, 3-[[4-[(2R)-3-(1-bicyclo[1.1.1]pentanyl)-2-[(6-cyclopropylfuro[2,3-b]pyrazin-2-yl)methylamino]propoxy]-6-(2,6-dimethylphenyl)pyrimidin-2-yl]sulfamoyl]benzoic acid (hydrochloride salt) (362 mg, 72%). ¹H NMR (400 MHz, DMSO-d6) δ 13.32 (s, 2H), 9.49 (s, 2H), 8.47 (t, J=1.9 Hz, 1H), 8.35 (s, 1H), 8.13 (d, J=8.2 Hz, 2H), 7.70 (t, J=7.8 Hz, 1H), 7.25 (d, J=7.8 Hz, 1H), 7.13 (d, J=7.6 Hz, 2H), 6.95 (s, 1H), 6.33 (s, 1H), 4.60-4.42 (m, 3H), 4.30 (d, J=11.1 Hz, 1H), 3.54 (s, 1H), 2.33-2.24 (m, 1H), 2.07-1.91 (m, 9H), 1.75-1.63 (m, 6H), 1.22-1.18 (m, 2H), 1.11-1.06 (m, 2H). ESI-MS m/z calc. 694.2573, found 695.7 (M+1)⁺; Retention time: 0.57 minutes; LC method B.

### Step 8: (11R)-11-(1-Bicyclo[0.1.]pentanylmethyl)-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-52

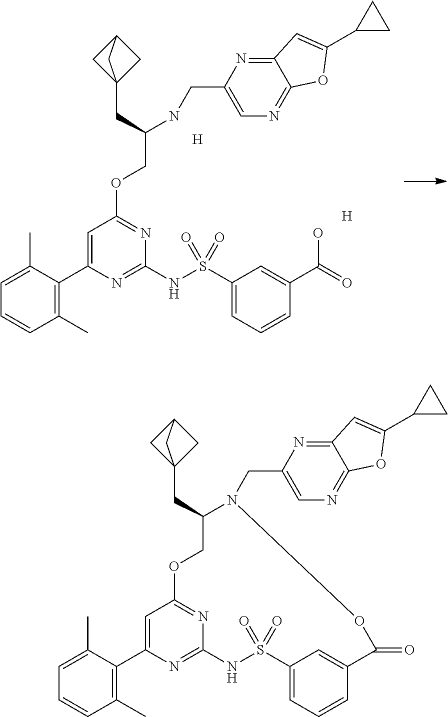

3-[[4-[(2R)-3-(1-bicyclo[1.1.1]pentanyl)-2-[(6-cyclopropylfuro[2,3-b]pyrazin-2-yl)methylamino]propoxy]-6-(2,6-dimethylphenyl)pyrimidin-2-yl]sulfamoyl]benzoic acid (hydrochloride salt) (362 mg, 0.4950 mmol) was combined with CDMT (108 mg, 0.6151 mmol) in anhydrous DMF (40 mL) and cooled to 0° C. N-methylmorpholine (330 μL, 3.002 mmol) was added and the reaction mixture was allowed to warm slowly to room temperature as the ice melted, stirring for a total of 18 hours. Volatiles were then removed under reduced pressure, and the resulting residue was dissolved in DMSO/methanol, filtered, and purified by preparative HPLC (1-99% ACN in water, HCl modifier, 15-minute run). Fractions containing product were combined, diluted slightly with brine, and extracted 2× with ethyl acetate. The combined organics were washed water and brine, then dried over sodium sulfate to give (11R)-11-(1-bicyclo[1.1.1]pentanylmethyl)-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (233.3 mg, 66%). ¹H NMR (400 MHz, Chloroform-d) δ 8.71 (t, J=1.8 Hz, 1H), 8.34 (s, 1H), 8.15 (d, J=8.0 Hz, 1H), 7.87 (dt, J=7.7, 1.4 Hz, 1H), 7.66 (t, J=7.8 Hz, 1H), 7.19 (t, J=7.6 Hz, 1H), 7.02 (d, J=7.7 Hz, 2H), 6.62 (s, 1H), 6.16 (s, 1H), 5.39 (dd, J=11.3, 3.9 Hz, 1H), 5.32 (d, J=15.1 Hz, 1H), 4.25-4.13 (m, 2H), 4.01 (tt, J=11.3, 3.6 Hz, 1H), 2.44 (s, 1H), 2.13 (tt, J=8.1, 5.3 Hz, 1H), 1.98 (s, 6H), 1.80 (dd, J=15.6, 3.3 Hz, 1H), 1.62-1.54 (m, 7H), 1.20-1.12 (m, 4H). ESI-MS m/z calc. 676.24677, found 677.5 (M+1)⁺; Retention time: 2.13 minutes; LC method A.

### Example 53: Preparation of Compound I-53

### Step 1: 2-Chloro-7-iodo-6-isopropyl-furo[2,3-b]pyrazine

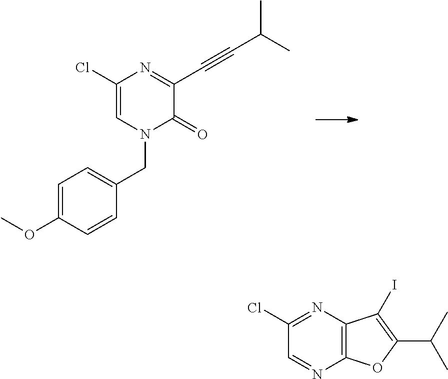 [SMILES: COc1ccc(Cn2cc(Cl)nc(C#CC(C)C)c2=O)cc1 ; CC(C)c1oc2ncc(Cl)nc2c1I]

To a solution of 5-chloro-1-[(4-methoxyphenyl)methyl]-3-(3-methylbut-1-ynyl)pyrazin-2-one (1.39 g, 4.3879 mmol) in dry DCM (28 mL), Iodine (1.4 g, 5.5160 mmol) in DCM (28 mL) was added slowly for 30 minutes and the resulting mixture was stirred at room temperature for 30 min. The solvent was evaporated and the residue was subjected to silica gel column chromatography in 0% to 10% ethyl acetate in hexanes. Collected fractions were combined and concentrated. The residue was triturated with hexane (10 mL) for 30 minutes. The precipitate was filtered and washed with hexane to afford 2-chloro-7-iodo-6-isopropyl-furo[2,3-b]pyrazine (1.1 g, 74%) as a pale yellow solid. ¹H NMR (500 MHz, CDCl₃) δ 8.15 (s, 1H), 3.43 (hept, J=6.9 Hz, 1H), 1.40 (d, J=6.9 Hz, 6H). ESI-MS m/z calc. 321.93698, found 323.1 (M+1)⁺; Retention time: 3.14 minutes; LC method E.

### Step 2: 2-Chloro-6-isopropyl-furo[2,3-b]pyrazine-7-carbonitrile

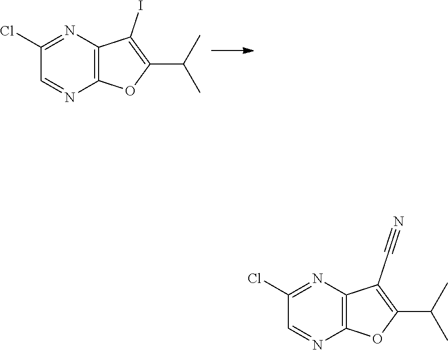

Under argon, to a microwave vial containing 2-chloro-7-iodo-6-isopropyl-furo[2,3-b]pyrazine (100 mg, 0.3100 mmol) and CuCN (55 mg, 0.6141 mmol) was added dry DMF (0.400 mL) and the mixture was heated at 130° C. for 1 h. The reaction mixture was further heated at 130° C. for 2 h. The reaction was cooled to room temperature and 1 M HCl (5 mL) was added. The aqueous phase was extracted with EtOAc (3×15 mL) and the combined organic extracts were washed with brine (2×65 mL), dried over MgSO₄, filtered and concentrated in vacuo. The residue was purified by flash chromatography (loaded onto a 12 g silica cartridge with benzene, and eluted with 0-7% EtOAc in hexanes over a 30 min gradient) to provide 2-chloro-6-isopropyl-furo[2,3-b]pyrazine-7-carbonitrile (20 mg, 29%) as a colorless solid. ¹H NMR (500 MHz, Chloroform-d) δ 8.32 (s, 1H), 3.51 (hept, J=7.0 Hz, 1H), 1.52 (d, J=7.0 Hz, 6H). ¹³C NMR (126 MHz, Chloroform-d) δ 178.21, 152.67, 147.06, 138.56, 137.86, 110.03, 90.23, 29.81, 20.26. ESI-MS m/z calc. 221.03558, found 222.2 (M+1)⁺; Retention time: 4.68 minutes; LC method D.

### Step 3: 6-Isopropyl-2-vinyl-furo[2,3-b]pyrazine-7-carbonitrile

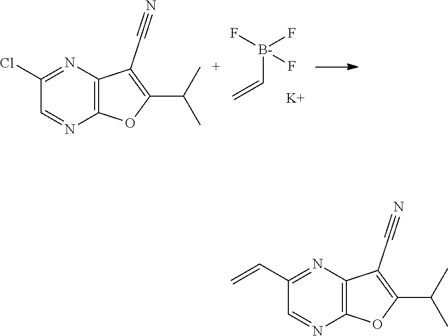

Under argon, 2-chloro-6-isopropyl-furo[2,3-b]pyrazine-7-carbonitrile (55 mg, 0.2481 mmol), potassium vinyltrifluoroborate (66.5 mg, 0.4965 mmol) Pd(PPh₃)₄ (28.6 mg, 0.0247 mmol) and K₃PO₄ (158 mg, 0.7443 mmol) were added to a microwave vial followed by degassed DMF (0.5 mL). The mixture was further degassed with an argon sparge for 5 minutes then sealed under argon and heated at 120° C. in a microwave reactor for 45 minutes. The reaction was cooled down to room temperature. Water and brine (1:1, 15 mL) were added and the reaction was extracted with EtOAc (3×30 mL). The organic phase was washed with brine (2×100 mL), dried over MgSO₄, filtered and concentrated in vacuo. The residue was purified by flash chromatography (loaded onto a 12 g silica cartridge with benzene, and eluted with 0-10% EtOAc in hexanes over a 40 min gradient) to provide 6-isopropyl-2-vinyl-furo[2,3-b]pyrazine-7-carbonitrile (15.5 mg, 29%) as a colorless solid. ¹H NMR (400 MHz, Chloroform-d) δ 8.30 (s, 1H), 6.92 (dd, J=17.4, 10.8 Hz, 1H), 6.46 (dd, J=17.4, 1.1 Hz, 1H), 5.65 (dd, J=10.8, 1.2 Hz, 1H), 3.49 (hept, J=7.0 Hz, 1H), 1.51 (d, J=7.0 Hz, 6H). ESI-MS m/z calc. 213.09021, found 214.1 (M+1)⁺; Retention time: 4.75 minutes; LC method D.

### Step 4: 2-Formyl-6-isopropyl-furo[2,3-b]pyrazine-7-carbonitrile

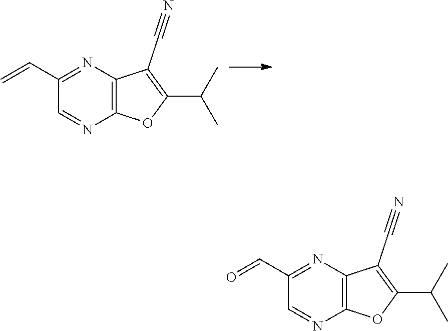 [SMILES: C=Cc1cnc2oc(C(C)C)c(C#N)c2n1 ; CC(C)c1oc2ncc(C=O)nc2c1C#N]

To a solution of 6-isopropyl-2-vinyl-furo[2,3-b]pyrazine-7-carbonitrile (45 mg, 0.2110 mmol) in dioxane (0.450 mL) and water (0.153 mL) was added pyridine (33.252 mg, 0.034 mL, 0.4204 mmol), OsO₄ in tBuOH (0.085 mL of 2.5% w/v, 0.0084 mmol) then NaIO₄ (180 mg, 0.8415 mmol) at room temperature. The reaction was stirred at room temperature for 1.5 h then and additional OsO₄ in tBuOH (0.540 mL of 2.5% w/v, 0.0531 mmol), pyridine (66.504 mg, 0.068 mL, 0.8408 mmol), dioxane (1 mL) and H₂O (0.3 mL) were added to the thick paste. The reaction was stirred or 4.5 h, diluted with DCM (30 mL) washed with sodium bicarbonate (20 mL), extracted with DCM (3×15 mL), washed with brine (50 mL) dried over sodium sulfate, filtered and concentrated in vacuo. The residue was purified by flash chromatography (loaded onto a 12 g silica cartridge with benzene, and eluted with 0-10% EtOAc in hexanes over a 45 min gradient) to provide 2-formyl-6-isopropyl-furo[2,3-b]pyrazine-7-carbonitrile (18 mg, 39%) as a colorless oil. ¹H NMR (500 MHz, Chloroform-d) δ 10.24 (s, 1H), 8.99 (s, 1H), 3.56 (hept, J=7.0 Hz, 1H), 1.55 (d, J=7.1 Hz, 6H). ESI-MS m/z calc. 215.06947, found 216.4 (M+1)⁺; Retention time: 1.9 minutes; LC method H.

### Step 5: 3-[[4-[(2R)-2-Amino-3-(1-methylcyclopropyl)propoxy]-6-(2,6-dimethylphenyl)pyrimidin-2-yl]sulfamoyl]benzoic acid

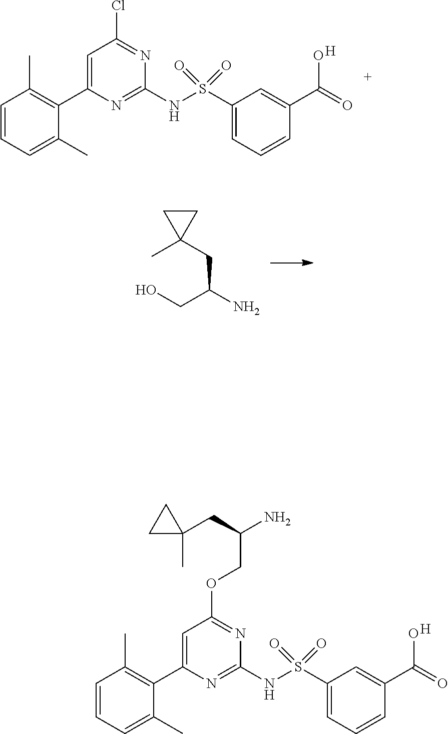

To a stirred mixture of 3-[[4-chloro-6-(2,6-dimethylphenyl)pyrimidin-2-yl]sulfamoyl]benzoic acid (2.60 g, 6.222 mmol) and (2R)-2-amino-3-(1-methylcyclopropyl) propan-1-ol (hydrochloride salt) (1.05 g, 6.338 mmol) in anhydrous tetrahydrofuran (25 mL) nitrogen was purged for 5 min. Then solid sodium tert-butoxide (2.50 g, 26.01 mmol) was added at once (mild exothermicity observed). The heterogeneous mixture was stirred at ambient temperature for 20 h. The reaction was quenched by addition of cold hydrochloric acid (35 mL of 1.0 M, 35.00 mmol) and extracted with ethyl acetate (3×25 mL). The combined organics were attempted to wash with brine (20 mL), and at that point some solid started to float in the organic layer. After removing the aqueous layer. the heterogeneous organic layer was concentrated under reduced pressure to furnish 3-[[4-[(2R)-2-amino-3-(1-methylcyclopropyl) propoxy]-6-(2,6-dimethylphenyl)pyrimidin-2-yl]sulfamoyl]benzoic acid (hydrochloride salt) (3.40 g, 99%) as a colorless solid. ESI-MS m/z calc. 510.1937, found 511.1 (M+1)⁺; Retention time: 1.05 minutes; LC method A.

### Step 6: 3-[[4-[(2R)-2-[(7-Cyano-6-isopropyl-furo[2,3-b]pyrazin-2-yl)methylamino]-3-(1-methylcyclopropyl)propoxy]-6-(2,6-dimethylphenyl)pyrimidin-2-yl]sulfamoyl]benzoic acid

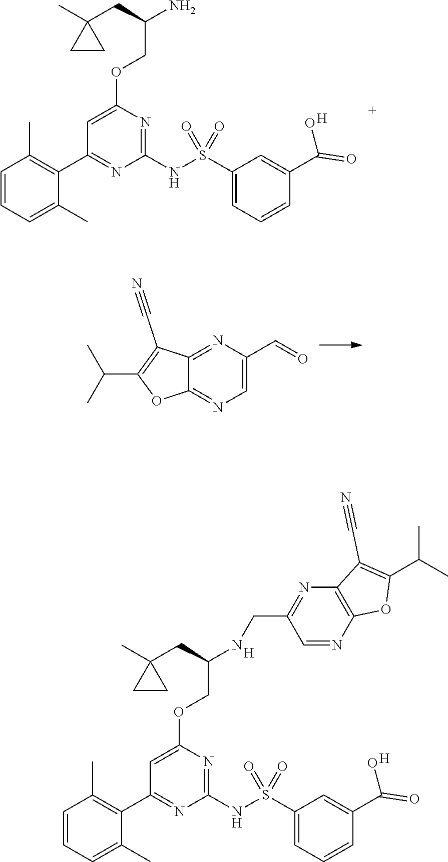

In a 4 mL vial, to a stirred mixture of 3-[[4-[(2R)-2-amino-3-(1-methylcyclopropyl) propoxy]-6-(2,6-dimethylphenyl)pyrimidin-2-yl]sulfamoyl]benzoic acid (hydrochloride salt) (45 mg, 0.08226 mmol) and 2-formyl-6-isopropyl-furo[2,3-b]pyrazine-7-carbonitrile (18 mg, 0.08364 mmol) in anhydrous dichloromethane (300 μL) were added glacial acetic acid (10 μL, 0.1758 mmol) and DIPEA (45 μL, 0.2584 mmol), in that order, at 0° C. under nitrogen. After 2 min, sodium triacetoxyborohydride (60 mg, 0.2831 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish product as a yellow solid. 3-[[4-[(2R)-2-[(7-cyano-6-isopropyl-furo[2,3-b]pyrazin-2-yl)methylamino]-3-(1-methylcyclopropyl)propoxy]-6-(2,6-dimethylphenyl)pyrimidin-2-yl]sulfamoyl]benzoic acid (hydrochloride salt) (38 mg, 62%). ESI-MS m/z calc. 709.26825, found 710.9 (M+1)⁺; Retention time: 1.43 minutes; LC method A.

### Step 7: 2-[[(11R)-6-(2,6-Dimethylphenyl)-11-[(1-methylcyclopropyl)methyl]-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-6-isopropyl-furo[2,3-b]pyrazine-7-carbonitrile (Compound I-53

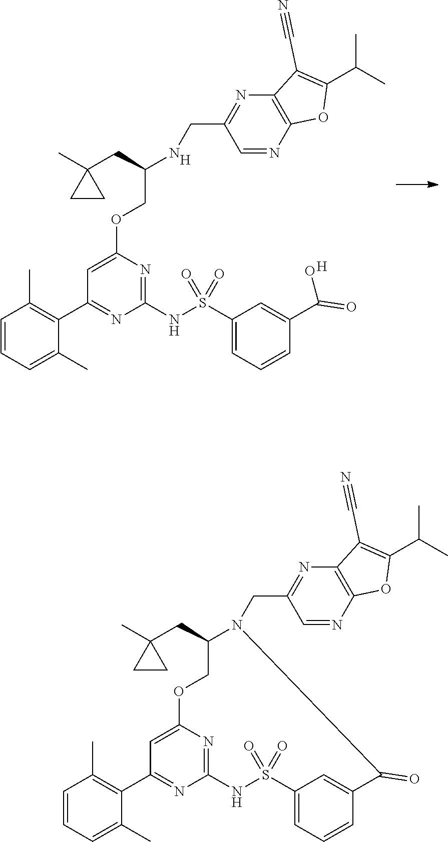

To a stirred solution of 3-[[4-[(2R)-2-[(7-cyano-6-isopropyl-furo[2,3-b]pyrazin-2-yl)methylamino]-3-(1-methylcyclopropyl)propoxy]-6-(2,6-dimethylphenyl)pyrimidin-2-yl]sulfamoyl]benzoic acid (hydrochloride salt) (38 mg, 0.05092 mmol) in anhydrous DMF (1.7 mL) was added 2-chloro-4,6-dimethoxy-1,3,5-triazine (14 mg, 0.07974 mmol) (CDMT), followed by addition of 4-methylmorpholine (40 μL, 0.3638 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished as a white solid. 2-[[(11R)-6-(2,6-dimethylphenyl)-11-[(1-methylcyclopropyl)methyl]-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-6-isopropyl-furo[2,3-b]pyrazine-7-carbonitrile (21 mg, 59%) ¹H NMR (400 MHz, CDCl₃) δ 9.57 (s, 1H), 8.73 (s, 1H), 8.53 (t, J=1.8 Hz, 1H), 8.12 (dt, J=8.3, 1.4 Hz, 1H), 7.84 (dt, J=7.8, 1.3 Hz, 1H), 7.62 (t, J=7.8 Hz, 1H), 7.20 (t, J=7.6 Hz, 1H), 7.04 (d, J=7.6 Hz, 2H), 6.22 (s, 1H), 5.28 (d, J=14.2 Hz, 1H), 5.22 (dd, J=11.0, 4.2 Hz, 1H), 4.53 (t, J=11.5 Hz, 1H), 4.47-4.37 (m, 1H), 4.20 (d, J=14.3 Hz, 1H), 3.52 (hept, J=7.0 Hz, 1H), 1.99 (s, 6H), 1.88-1.80 (m, 1H), 1.56 (s, 1H), 1.54 (d, J=6.7 Hz, 3H), 1.53 (d, J=6.8 Hz, 3H), 0.47 (s, 3H), 0.37-0.29 (m, 1H), 0.29-0.20 (m, 1H), 0.19-0.09 (m, 1H), 0.07-0.01 (m, 1H). ESI-MS m/z calc. 691.2577, found 692.8 (M+1)⁺; Retention time: 2.0 minutes; LC method A.

### Example 54: Preparation of Compound I-62

### Step 1: Methyl 7-bromo-6-isopropyl-furo[2,3-b]pyrazine-2-carboxylate

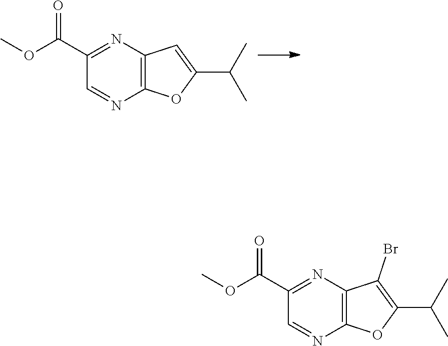 [SMILES: COC(=O)c1cnc2oc(C(C)C)cc2n1 ; COC(=O)c1cnc2oc(C(C)C)c(Br)c2n1]

To a solution of methyl 6-isopropylfuro[2,3-b]pyrazine-2-carboxylate (2.98 g, 13.532 mmol) in DMF (60 mL) at 0° C. was added NBS (7.3 g, 41.015 mmol). The reaction mixture was stirred at 60° C. for 4 h. The reaction mixture was quenched with sodium bicarbonate (40 mL), diluted with EtOAc (200 mL), washed with a sodium bisulfate solution (80 mL), water (3×80 mL), dried over sodium sulfate, filtered, and concentrated in vacuo. The residue was purified by silica gel chromatography using 0-40% ethyl acetate in hexanes to furnish methyl 7-bromo-6-isopropyl-furo[2,3-b]pyrazine-2-carboxylate (3.312 g, 82%) as white solid. ¹H NMR (500 MHz, Chloroform-d) δ 9.05 (s, 1H), 4.06 (s, 3H), 3.49 (hept, J=7.0 Hz, 1H), 1.43 (d, J=7.0 Hz, 6H). ESI-MS m/z calc. 297.9953, found 299.4 (M+1)⁺; Retention time: 2.73 minutes; LC method E.

### Step 2: Methyl 6-isopropyl-7-methyl-furo[2,3-b]pyrazine-2-carboxylate

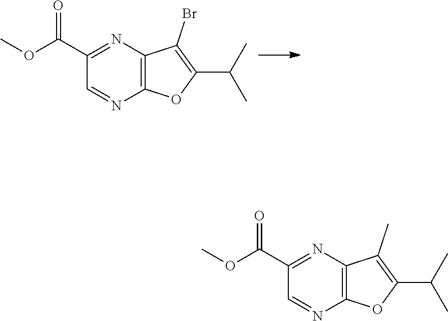 [SMILES: COC(=O)c1cnc2oc(C(C)C)c(Br)c2n1 ; COC(=O)c1cnc2oc(C(C)C)c(C)c2n1]

Under argon, to a vial containing methyl 7-bromo-6-isopropyl-furo[2,3-b]pyrazine-2-carboxylate (300 mg, 1.0029 mmol) was added K₃PO₄ (638 mg, 3.0057 mmol), Pd(PPh₃)₄ (115 mg, 0.0995 mmol) and trimethylboroxine (257.40 mg, 0.300 mL, 2.0504 mmol), degassed DMF (12 mL), followed by degassed water (3 mL). The mixture was sealed under argon and heated at 180° C. for 30 minutes in a microwave reactor. To the reaction was added 1M NaOH (15 mL, until pH=10), and the aqueous phase was washed with diethyl ether (25 mL), EtOAc (25 mL). The aqueous phase was acidified with 3 M HCl (until pH=3), extracted with EtOAc (3×25 mL), washed with acidic brine (150 mL, pH=3), dried over MgSO₄, filtered and concentrated in vacuo to provide 6-isopropyl-7-methyl-furo[2,3-b]pyrazine-2-carboxylic acid (260 mg) as an amber oil. ¹H NMR (500 MHz, Chloroform-d) δ 9.07 (s, 1H), 3.34 (m, 1H), 2.30 (s, 3H), 1.41 (d, J=7.0 Hz, 6H). The residue was dissolved in DMF (4 mL) and potassium carbonate (416 mg, 3.0100 mmol) then Mel (426.36 mg, 0.187 mL, 3.0038 mmol) were added, and the reaction was stirred for 45 minutes. Additional potassium carbonate (140 mg, 1.0130 mmol) and Mel (159.60 mg, 0.070 mL, 1.1244 mmol) were added and the reaction was stirred for 30 minutes. The reaction was quenched with saturated aqueous ammonium chloride (15 mL, after addition pH=7), extracted with EtOAc (3×30 mL). The combined organics were washed with ammonium chloride (15 mL), brine (2×100 mL), dried over MgSO₄, filtered and concentrated in vacuo. The residue (160 mg) was purified by flash chromatography (loaded onto a 12 g silica cartridge with benzene, and eluted with 0-40% diethyl ether in hexanes over a 30 min gradient) to provide methyl 6-isopropyl-7-methyl-furo[2,3-b]pyrazine-2-carboxylate (120 mg, 51%) as a white solid. ¹H NMR (500 MHz, Chloroform-d) δ 8.97 (s, 1H), 4.04 (s, 3H), 3.31 (hept, J=7.0 Hz, 1H), 2.31 (s, 3H), 1.39 (d, J=7.0 Hz, 6H). ESI-MS m/z calc. 234.10045, found 235.2 (M+1)⁺; Retention time: 4.3 minutes; LC method D.

### Step 3: 6-Isopropyl-7-methyl-furo[2,3-b]pyrazine-2-carbaldehyde

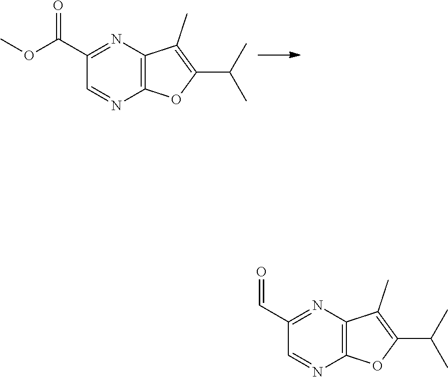 [SMILES: COC(=O)c1cnc2oc(C(C)C)c(C)c2n1 ; Cc1c(C(C)C)oc2ncc(C=O)nc12]

A solution of methyl 6-isopropyl-7-methyl-furo[2,3-b]pyrazine-2-carboxylate (384 mg, 1.6393 mmol) in DCM (5.8 mL) was cooled in a −78° C. bath then a solution of DIBAL in DCM (3 mL of 1 M, 3.0000 mmol) was added dropwise. The reaction was stirred for 2 h at −78° C. The reaction was quenched with a MeOH—H₂O solution (1:1; 10 mL) at −78° C. and warmed to room temperature and then stirred for 1 h. The reaction was concentrated in vacuo. The residue was diluted with DCM (30 mL) and 1 M HCl (35 mL), the aqueous phase was extracted with DCM (3×30 mL). The combined organic phases were washed with brine (75 mL), dried over MgSO₄, filtered and concentrated in vacuo to provide 6-isopropyl-7-methyl-furo[2,3-b]pyrazine-2-carbaldehyde (293 mg, 85%) as a pale yellow solid. ¹H NMR (500 MHz, Chloroform-d) δ 10.22 (s, 1H), 8.84 (s, 1H), 3.33 (hept, J=7.0 Hz, 1H), 2.32 (s, 3H), 1.40 (d, J=7.0 Hz, 6H). ESI-MS m/z calc. 204.08987, found 205.0 (M+1)⁺; Retention time: 2.15 minutes; LC method H.

### Step 3: 3-[[4-[(2R)-2-Amino-3-(1-bicyclo[1.1.1]pentanyl)propoxy]-6-(2,6-dimethylphenyl)pyrimidin-2-yl]sulfamoyl]benzoic acid

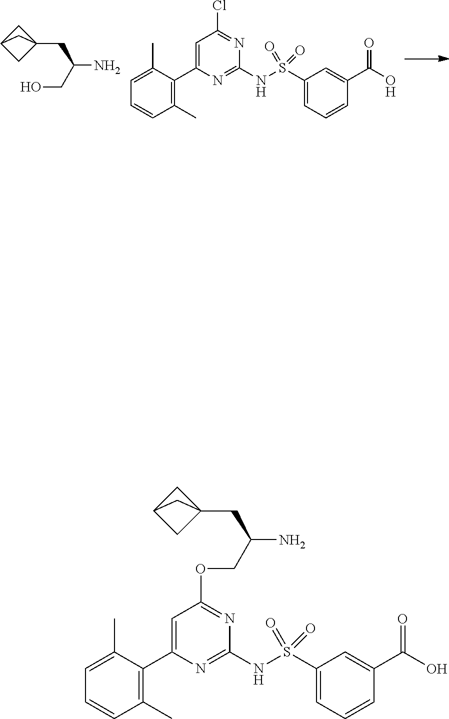 [SMILES: N[C@@H](CO)CC12CC(C1)C2 ; Cc1cccc(C)c1-c1cc(Cl)nc(NS(=O)(=O)c2cccc(C(=O)O)c2)n1 ; Cc1cccc(C)c1-c1cc(OC[C@H](N)CC23CC(C2)C3)nc(NS(=O)(=O)c2cccc(C(=O)O)c2)n1]

A flame-dried flask under nitrogen atmosphere was charged with (2R)-2-amino-3-(1-bicyclo[1.1.1]pentanyl)propan-1-ol (hydrochloride salt) (505 mg, 2.7002 mmol), 3-[[4-chloro-6-(2,6-dimethylphenyl)pyrimidin-2-yl]sulfamoyl]benzoic acid (1.3 g, 3.1110 mmol), DMF (5.8 mL) and MeTHF (58 mL). This suspension was cooled down to 0° C. and sodium tert-butoxide (1.5 g, 15.608 mmol) was added and left stirring for 5 min at this temperature. The reaction was then left to warm-up to room temperature and stirred 30 min. The mixture was then cooled down to 0° C. and diluted with MeTHF (50 mL) quenched with ice cold 1 N HCl (100 mL) while stirring vigorously. The aqueous phase was separated and extracted with MeTHF (3×50 mL). The combined organic phases were dried with sodium sulfate, filtered, and concentrated under reduced pressure. The resulting residue was purified by reverse phase chromatography on 100 g C₁₈ aq. RediSep Rf gold column using a 5 to 100% gradient of acetonitrile in acidic water (0.1% HCl content) to provide 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (1.254 g, 82%) as a white powder. ¹H NMR (400 MHz, DMSO-d6) δ 13.07 (br. s, 1H), 8.46 (t, J=1.7 Hz, 1H), 8.25 (br. s, 3H), 8.14 (dd, J=14.1, 7.7 Hz, 2H), 7.71 (t, J=7.7 Hz, 1H), 7.31-7.21 (m, 1H), 7.18-7.07 (m, 2H), 6.30 (br. s, 1H), 4.35 (dd, J=11.7, 2.2 Hz, 1H), 4.16 (dd, J=12.0, 6.6 Hz, 1H), 3.49-3.45 (m, 1H, overlap with water), 2.46 (s, 1H), 2.07 (s, 1H), 2.01 (br. s, 6H), 1.83 (d, J=6.8 Hz, 2H), 1.75, 1.70 (ABq·JAB=9.5 Hz, 6H). ESI-MS m/z calc. 522.19366, found 523.2 (M+1)⁺; Retention time: 2.48 minutes; LC method J.

### Step 4: 3-[[4-[(2R)-3-(1-Bicyclo[1.1.1]pentanyl)-2-[(6-isopropyl-7-methyl-furo[2,3-b]pyrazin-2-yl)methylamino]propoxy]-6-(2,6-dimethylphenyl)pyrimidin-2-yl]sulfamoyl]benzoic acid

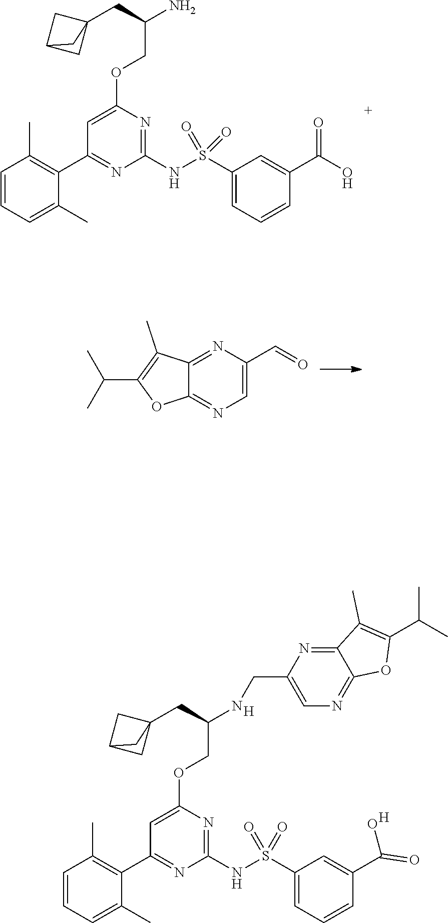

A 4 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (62 mg, 0.1102 mmol), 6-isopropyl-7-methyl-furo[2,3-b]pyrazine-2-carbaldehyde (24.0 mg, 0.1139 mmol), anhydrous DCM (500 μL), acetic acid (16 μL, 0.2814 mmol) and DIEA (68 μL, 0.3904 mmol). The mixture was cooled down in an ice bath, sodium triacetoxyborohydride (82 mg, 0.3869 mmol) was added and the reaction was stirred for 1.5 h. The reaction was quenched with aqueous 1 M HCl (1 mL), MeOH (0.5 mL) and DMSO (0.5 mL), filtered and purified by reverse phase HPLC (1-70% acetonitrile/5 mM aqueous HCl over 30 min) to give as a white solid 3-[[4-[(2R)-3-(1-bicyclo[1.1.1]pentanyl)-2-[(6-isopropyl-7-methyl-furo[2,3-b]pyrazin-2-yl)methylamino]propoxy]-6-(2,6-dimethylphenyl)pyrimidin-2-yl]sulfamoyl]benzoic acid (hydrochloride salt) (59.8 mg, 73%). ESI-MS m/z calc. 710.28864, found 711.6 (M+1)⁺; Retention time: 1.62 minutes. LC method A.

### Step 5: (11R)-11-(1-Bicyclo[1.1.]pentanylmethyl)-6-(2,6-dimethylphenyl)-12-[(6-isopropyl-7-methyl-furo[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-62

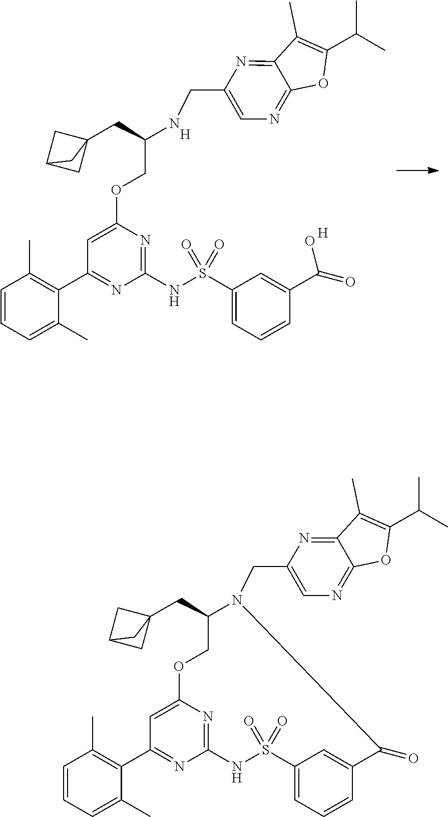

3-[[4-[(2R)-3-(1-bicyclo[1.1.1]pentanyl)-2-[(6-isopropyl-7-methyl-furo[2,3-b]pyrazin-2-yl)methylamino]propoxy]-6-(2,6-dimethylphenyl)pyrimidin-2-yl]sulfamoyl]benzoic acid (hydrochloride salt) (59 mg, 0.07895 mmol) was combined under nitrogen with CDMT (37 mg, 0.2107 mmol) and DMF (2.0 mL). The solution was stirred at 0° C. 4-Methyl-morpholine (55 μL, 0.5003 mmol) was added and the mixture was stirred in the cooling bath that was allowed to warm to room temperature. After 14 h, the reaction was filtered and purified by reverse phase HPLC (30-99% acetonitrile/5 mM aqueous HCl over 30 min) to give as a white solid (11R)-11-(1-bicyclo[1.1.1]pentanylmethyl)-6-(2,6-dimethylphenyl)-12-[(6-isopropyl-7-methyl-furo[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (38.5 mg, 70%), ¹H NMR (400 MHz, Chloroform-d) δ 9.09 (s, 1H), 8.81 (t, J=1.9 Hz, 1H), 8.34 (s, 1H), 8.05 (d, J=7.9 Hz, 1H), 7.89 (dt, J=7.7, 1.4 Hz, 1H), 7.65 (t, J=7.8 Hz, 1H), 7.21 (t, J=7.6 Hz, 1H), 7.05 (d, J=7.6 Hz, 2H), 6.21 (s, 1H), 5.46 (dd, J=11.2, 3.8 Hz, 1H), 5.35 (d, J=15.2 Hz, 1H), 4.26 (d, J=15.2 Hz, 1H), 4.16 (t, J=11.4 Hz, 1H), 4.10-3.99 (m, 1H), 3.33-3.20 (m, 1H), 2.45 (s, 1H), 2.30 (s, 3H), 2.03 (s, 6H), 1.81 (dd, J=15.5, 3.2 Hz, 1H), 1.56 (d, J=9.1 Hz, 6H), 1.37 (dd, J=6.9, 5.1 Hz, 6H), 0.89-0.76 (m, 1H). ESI-MS m/z calc. 692.2781, found 693.2 (M+1)⁺; Retention time: 2.3 minutes. LC method A.

### Example 55: Preparation of Compound I-66 and Compound I-67

### Step 1: Methyl 2-(tert-butoxycarbonylamino)-3-tetrahydropyran-3-yl-prop-2-enoate

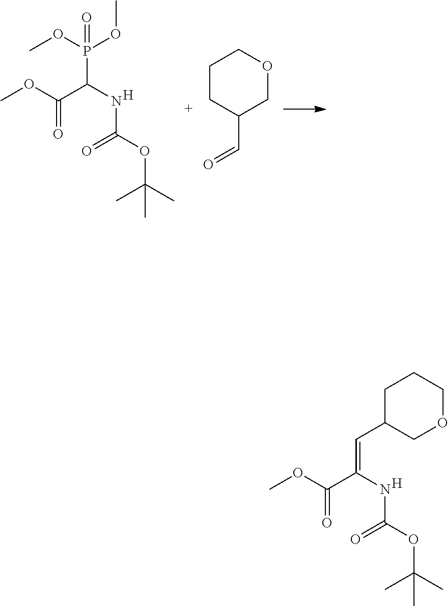

To a flask containing tetrahydropyran-3-carbaldehyde (8.08 g, 70.789 mmol) was added methyl 2-(tert-butoxycarbonylamino)-2-dimethoxyphosphoryl-acetate (20 g, 67.285 mmol) then THE (66 mL) and the mixture was cooled to −84° C. (liquid N₂-EtOAc). Then 1,1,3,3-Tetramethylguanidine (15.514 g, 16.9 mL, 134.70 mmol) was added dropwise over 1 minutes and the mixture was warmed to 0° C. over 2.5 h and then tetrahydropyran-3-carbaldehyde (400 mg, 3.5044 mmol) was added. The reaction was quenched with ammonium chloride (100 mL) at 0° C., diluted with water (50 mL), extracted with EtOAc (3×300 mL). The organic phase was washed with water (100 mL), brine (100 mL), dried over sodium sulfate, filtered and concentrated in vacuo. The residue was purified by flash chromatography (loaded onto a 220 g silica cartridge with benzene, and eluted with 0-30% EtOAc in hexanes over a 30 min gradient) to provide methyl 2-(tert-butoxycarbonylamino)-3-tetrahydropyran-3-yl-prop-2-enoate (19.2 g, 95%) as colorless crystals. ¹H NMR (500 MHz, Chloroform-d) δ 6.33 (d, J=10.1 Hz, 1H), 6.05-5.90 (m, 1H), 3.88-3.80 (m, 2H), 3.77 (s, 3H), 3.50-3.40 (m, 1H), 3.28 (dd, J=11.2, 9.2 Hz, 1H), 2.77-2.67 (m, 1H), 1.97-1.87 (m, 1H), 1.67-1.61 (m, 2H), 1.46 (s, 9H), 1.44-1.38 (m, 1H). ESI-MS m/z calc. 285.15762, found 286.3 (M+1)⁺; Retention time: 3.7 minutes; LC method D.

### Step 2: Methyl (2R)-2-(tert-butoxycarbonylamino)-3-tetrahydropyran-3-yl-propanoate

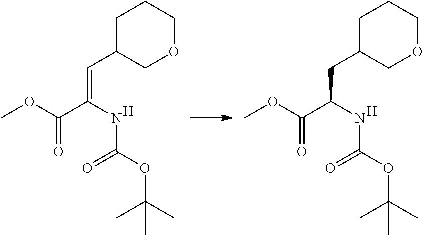

A solution of methyl (Z)-2-(tert-butoxycarbonylamino)-3-tetrahydropyran-3-yl-prop-2-enoate (13.81 g, 48.399 mmol) in EtOH (166 mL) and dioxane (55 mL) was sparged with nitrogen for 30 minutes, then 1,2-bis[(2R,5R)-2,5-diethylphospholano]benzene(1,5-cyclooctadiene)rhodium(I) trifluoromethanesulfonate [CAS #136705-77-6](434 mg, 0.6006 mmol) weighed out in a glove box into a 20 mL scintillation vial was then transferred under nitrogen in a vessel quickly pressurized with Hydrogen gas (3.5 bar; 60 psi) and vented 3 times, then pressurized again with hydrogen gas (60 psi) and reacted on a pressure shaker for 62 h at room temperature. The reaction mixture was filtered through silica (2.5×9 cm) and the filter was rinsed with EtOAc (5×100 mL). The filtrate was concentrated in vacuo and the residue was purified by flash chromatography (loaded onto a 220 g silica cartridge with benzene, and eluted with 0-20% Acetone in hexanes over a 60 min gradient) to provide methyl (2R)-2-(tert-butoxycarbonylamino)-3-tetrahydropyran-3-yl-propanoate (13.21 g, 95%) as a colorless oil that crystallized on standing. ¹H NMR (500 MHz, Chloroform-d) δ 5.00-4.86 (m, 1H), 4.39-4.24 (m, 1H), 3.89-3.78 (m, 2H), 3.74 (s, 3H), 3.41-3.34 (m, 1H), 3.14-3.05 (m, 1H), 2.01-1.82 (m, 1H), 1.74-1.67 (m, 1H), 1.64-1.55 (m, 3H), 1.44 (s, 10H), 1.22-1.15 (m, 1H). ESI-MS m/z calc. 287.17328, found 288.4 (M+1)⁺; Retention time: 3.76 minutes; LC method D.

### Step 3: tert-Butyl N-[(1R)-1-(hydroxymethyl)-2-tetrahydropyran-3-yl-ethyl]carbamate

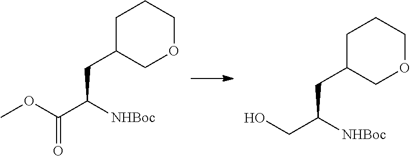 [SMILES: COC(=O)[C@@H](CC1CCCOC1)NC(=O)OC(C)(C)C ; CC(C)(C)OC(=O)N[C@@H](CO)CC1CCCOC1]

A solution of methyl (2R)-2-(tert-butoxycarbonylamino)-3-tetrahydropyran-3-yl-propanoate (13.21 g, 45.971 mmol) in THE (130 mL) was cooled to 0° C. then added dropwise a solution of LiBH₄ (2M in THF) (46 mL of 2 M, 92.0 mmol) over 5 minutes. The reaction was stirred at 0° C. for 30 minutes then warmed to room temperature and stirred 3.5 h. The reaction was cooled in a 0° C. then poured into cold saturated aqueous ammonium chloride (350 mL) and stirred vigorously until gas evolution ceased. The organics were extracted with EtOAc (3×350 mL), washed with brine, dried over sodium sulfate, filtered, and concentrated in vacuo. The residue was purified by flash chromatography (loaded onto a 220 g silica cartridge with benzene, and eluted with 0-60% acetone in hexanes over a 45 min gradient) to provide tert-butyl N-[(1R)-1-(hydroxymethyl)-2-tetrahydropyran-3-yl-ethyl]carbamate (11.35 g, 95%) as a thick colorless gel as a mixture of diastereomers. ESI-MS m/z calc. 259.1784, found 260.6 (M+1)⁺; Retention time: 2.85 minutes and 2.92 minutes. LC method D.

### Step 4: (2R)-2-Amino-3-tetrahydropyran-3-yl-propan-1-ol

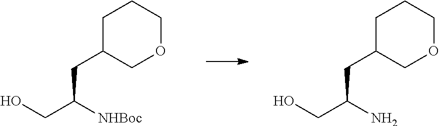 [SMILES: CC(C)(C)OC(=O)N[C@@H](CO)CC1CCCOC1 ; N[C@@H](CO)CC1CCCOC1]

To a solution of tert-butyl N-[(1R)-1-(hydroxymethyl)-2-tetrahydropyran-3-yl-ethyl]carbamate (7.13 g, 27.493 mmol) in was added HCl 4M in dioxane/water (62 mL of 4 M, 248.00 mmol) at room temperature and the reaction was stirred 13 h at room temperature. The reaction was concentrated in vacuo (65° C.) to provide (2R)-2-amino-3-tetrahydropyran-3-yl-propan-1-ol (hydrochloride salt) (5.38 g, 95%) as a sticky off white solid. ESI-MS m/z calc. 159.12593, found 160.4 (M+1)⁺; Retention time: 0.9 minutes; LC method D.

### Step 5: 3-[[4-[(2R)-2-Amino-3-tetrahydropyran-3-yl-propoxy]-6-(2,6-dimethylphenyl)pyrimidin-2-yl]sulfamoyl]benzoic acid

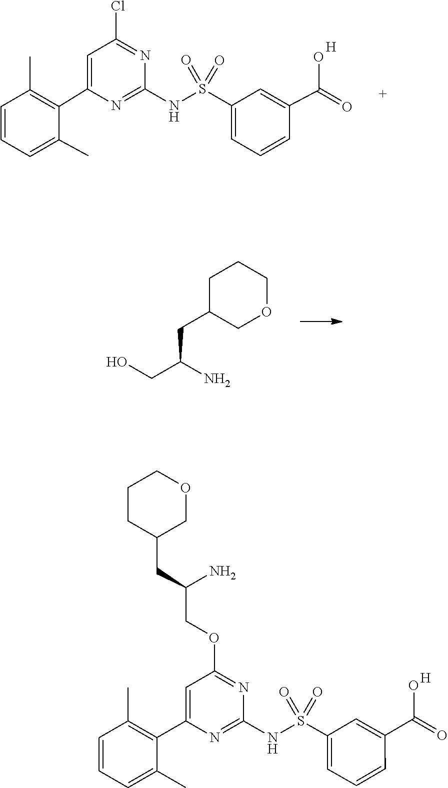

To a suspension of (2R)-2-amino-3-tetrahydropyran-3-yl-propan-1-ol (hydrochloride salt) (2.5 g, 12.776 mmol) in THE (65 mL) was added NaOtBu (7.36 g, 76.584 mmol) at room temperature, and the reaction was stirred for 15 minutes, then the reaction was cooled to 0° C. and stirred for another 15 minutes. Then 3-[[4-chloro-6-(2,6-dimethylphenyl)pyrimidin-2-yl]sulfamoyl]benzoic acid (5.47 g, 13.090 mmol) was added at 0° C. and the reaction was stirred 30 minutes, then warmed to room temperature. The reaction was stirred for 3 h at room temperature, then additional NaOtBu (1.2 g, 12.487 mmol) was added and there reaction was stirred for 30 minutes, then cooled to 0° C. and quenched with 3 M HCl (42 mL, until pH=1), extracted with EtOAc (4×150 mL). The organic phase was washed with brine (35 mL), dried over MgSO₄, filtered and concentrated in vacuo to a a low volume and the precipitate that formed was collected by vacuum filtration to provide 3-[[4-[(2R)-2-amino-3-tetrahydropyran-3-yl-propoxy]-6-(2,6-dimethylphenyl)pyrimidin-2-yl]sulfamoyl]benzoic acid (hydrochloride salt) (5.21 g, 69%) as a light pink solid ESI-MS m/z calc. 540.2043, found 541.3 (M+1)⁺; Retention time: 1.51 minutes and 1.52 minutes. LC method H.

### Step 6: 3-[[4-(2,6-Dimethylphenyl)-6-[(2R)-2-[(6-isopropylfuro[2,3-b]pyrazin-2-yl)methylamino]-3-tetrahydropyran-3-yl-propoxy]pyrimidin-2-yl]sulfamoyl]benzoic acid

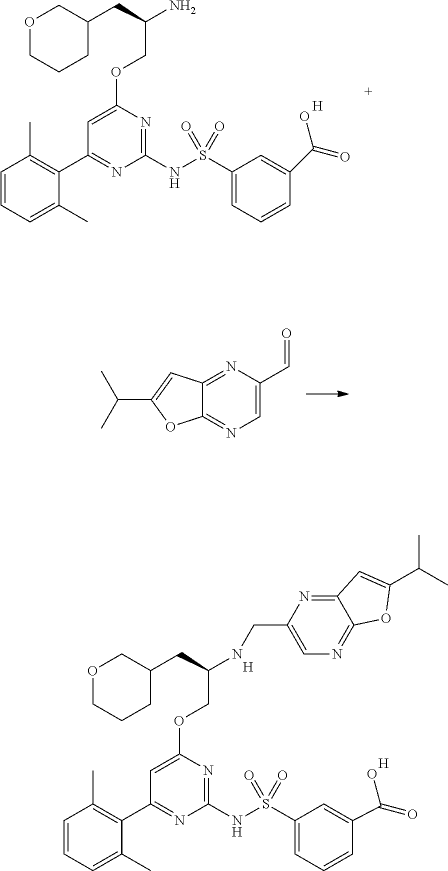

A 4 mL vial was charged under nitrogen with 3-[[4-[(2R)-2-amino-3-tetrahydropyran-3-yl-propoxy]-6-(2,6-dimethylphenyl)pyrimidin-2-yl]sulfamoyl]benzoic acid (hydrochloride salt) (100 mg, 0.1733 mmol), 6-isopropylfuro[2,3-b]pyrazine-2-carbaldehyde (33.6 mg, 0.1767 mmol), anhydrous DCM (1.3 mL), and acetic acid (0.01 mL, 0.1758 mmol). The mixture was cooled down in an ice bath. DIEA (0.04 mL, 0.2296 mmol) was added, followed by sodium triacetoxyborohydride (Sodium salt) (177.8 mg, 0.8389 mmol) and the reaction was vigorously stirred at 0° C. for 1 h. The reaction was quenched with 3 N aqueous HCl, diluted with MeOH and DMSO, and the resulting solution was filtered, and purified by reverse phase HPLC (1-99% acetonitrile/5 mM aqueous HCl over 15 min). The pure fractions were collected, extracted with EtOAc, dried over MgSO₄, filtered, and concentrated under reduced pressure to give 3-[[4-(2,6-dimethylphenyl)-6-[(2R)-2-[(6-isopropylfuro[2,3-b]pyrazin-2-yl)methylamino]-3-tetrahydropyran-3-yl-propoxy]pyrimidin-2-yl]sulfamoyl]benzoic acid (hydrochloride salt) (100.6 mg, 75%) as a white solid. ESI-MS m/z calc. 714.28357, found 715.4 (M+1)⁺; Retention time: 1.39 minutes. LC method A.

### Step 7: (11R)-6-(2,6-Dimethylphenyl)-12-[(6-isopropylfuro[2,3-b]pyrazin-2-yl)methyl]-2,2-dioxo-11-(tetrahydropyran-3-ylmethyl)-9-oxa-2λ⁶-thia-3,5,12,19-tetrazatricyclo[12.3.1.14,8]nonadeca-1(18),4(19),5,7,4,16-hexaen-13-one, SFC peak 1, Compound I-66, and (11R)-6-(2,6-dimethylphenyl)-12-[(6-isopropylfuro[2,3-b]pyrazin-2-yl)methyl]-2,2-dioxo-11-(tetrahydropyran-3-ylmethyl)-9-oxa-2λ⁶-thia-3,5,12,19-tetrazatricyclo[12.3.1.14,8]nonadeca-1(18),4(19),5,7,4,16-hexaen-13-one, SFC peak 2, Compound I-67

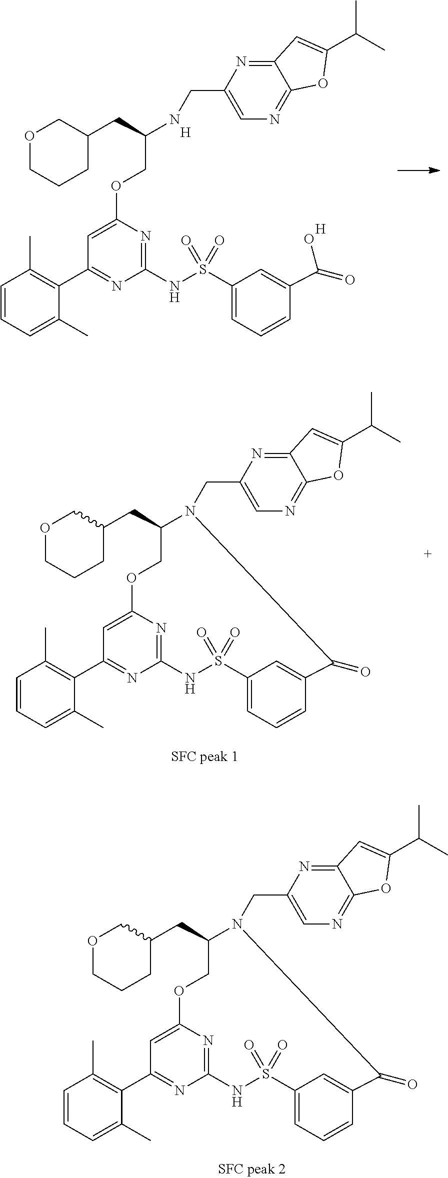

3-[[4-(2,6-dimethylphenyl)-6-[(2R)-2-[(6-isopropylfuro[2,3-b]pyrazin-2-yl)methylamino]-3-tetrahydropyran-3-yl-propoxy]pyrimidin-2-yl]sulfamoyl]benzoic acid (hydrochloride salt) (100.6 mg, 0.1299 mmol) was combined with CDMT (70.1 mg, 0.3993 mmol) in DMF (11.0 mL) and cooled to 0° C. N-methylmorpholine (0.07 mL, 0.6367 mmol) was added by syringe and the reaction was stirred at 0° C. for 30 minutes. The ice bath was then removed and stirring was continued for an additional 72 hours at room temperature. The reaction mixture was then partitioned between 50 mL 1 M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated to give 62.5 mg of a white solid which was submitted for SFC separation. This material was subjected to chiral SFC separation using a ChiralCel OD (4.6×250 mm, 5 m) at 40° C. The mobile phase was 26% MeOH (20 mM NH₃) at 70 mL/min flow in isocratic mode. Concentration of the sample was 25 mg/mL in MeOH:DMSO (5:1). The injection volume was 500 μL with an outlet pressure of 160 bar and the detection wavelength was 210 nm. For each isomer, the solvents were evaporated. The product was dissolved in DMSO (1 mL) and MeOH (1 mL) and purified by reverse phase HPLC (1-99% acetonitrile/5 mM aqueous HCl over 20 min) to give as white solids: Isomer 1, SFC peak 1 (11R)-6-(2,6-dimethylphenyl)-12-[(6-isopropylfuro[2,3-b]pyrazin-2-yl)methyl]-2,2-dioxo-11-(tetrahydropyran-3-ylmethyl)-9-oxa-2λ⁶-thia-3,5,12,19-tetrazatricyclo[12.3.1.14,8]nonadeca-1(18),4(19),5,7,14,16-hexaen-13-one (18.5 mg, 20%). ¹H NMR (400 MHz, CDCl₃) δ 8.63 (t, J=1.8 Hz, 1H), 8.41 (s, 1H), 7.84-7.77 (m, 2H), 7.59 (t, J=7.8 Hz, 1H), 7.22 (t, J=7.6 Hz, 1H), 7.05 (d, J=7.6 Hz, 2H), 6.62 (d, J=1.0 Hz, 1H), 6.20 (s, 1H), 5.38 (dd, J=11.3, 4.1 Hz, 1H), 5.32 (d, J=14.9 Hz, 1H), 4.33-4.19 (m, 2H), 4.11-4.01 (m, 1H), 3.78-3.71 (m, 1H), 3.30-3.21 (m, 2H), 3.21-3.11 (m, 1H), 2.67 (dd, J=11.2, 8.9 Hz, 1H), 2.02 (s, 6H), 1.74-1.58 (m, 4H), 1.52-1.43 (m, 2H), 1.40 (d, J=6.9 Hz, 6H), 1.27-1.10 (m, 1H). ESI-MS m/z calc. 696.273, found 697.4 (M+1)⁺; Retention time: 1.96 minutes. and Isomer 2, SFC peak 2 (11R)-6-(2,6-dimethylphenyl)-12-[(6-isopropylfuro[2,3-b]pyrazin-2-yl)methyl]-2,2-dioxo-11-(tetrahydropyran-3-ylmethyl)-9-oxa-2λ⁶-thia-3,5,12,19-tetrazatricyclo[12.3.1.14,8]nonadeca-1(18),4(19),5,7,14,16-hexaen-13-one (15.0 mg, 17%). ¹H NMR (400 MHz, CDCl₃) δ 8.68 (t, J=1.8 Hz, 1H), 8.41 (s, 1H), 8.21-8.16 (m, 1H), 7.80 (dt, J=7.7, 1.3 Hz, 1H), 7.66 (t, J=7.8 Hz, 1H), 7.19 (t, J=7.6 Hz, 1H), 7.01 (d, J=7.6 Hz, 2H), 6.62 (d, J=1.0 Hz, 1H), 6.17 (s, 1H), 5.41 (dd, J=11.1, 3.9 Hz, 1H), 5.31 (d, J=14.9 Hz, 1H), 4.32-4.23 (m, 2H), 4.21-4.11 (m, 1H), 3.81-3.69 (m, 2H), 3.32-3.23 (m, 1H), 3.21-3.12 (m, 1H), 3.05 (dd, J=11.1, 8.7 Hz, 1H), 1.96 (s, 6H), 1.70 (t, J=11.4 Hz, 2H), 1.46-1.42 (m, 1H), 1.42-1.35 (m, 8H), 1.26-1.18 (m, 1H), 0.74-0.62 (m, 1H). ESI-MS m/z calc. 696.273, found 697.4 (M+1)⁺; Retention time: 1.92 minutes. LC method A.

### Example 56: Preparation of Compound I-71

### Step 1: Benzyl N-[1-(hydroxymethyl)-3-(1-methylcyclopropyl)propyl]carbamate

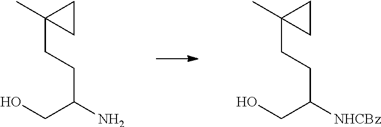

To a stirred solution of 2-amino-4-(1-methylcyclopropyl)butan-1-ol (hydrochloride salt) (2.235 g, 11.816 mmol) and 2-amino-4-(1-methylcyclopropyl)butan-1-ol (hydrochloride salt) (1.291 g, 6.8255 mmol) in a 1:1 mixture of tetrahydrofuran (100 mL) and water (100 mL) were added sodium bicarbonate (5 g, 59.519 mmol) and N-(benzyloxycarbonyloxy)succinimide (5.9 g, 23.674 mmol). The reaction was stirred at room temperature for 3 hours. The reaction was then stopped by the addition of a saturated aqueous solution of ammonium chloride (50 mL). The layers were separated, and the aqueous layer was extracted with ethyl acetate (5×100 mL). The combined organic layers were then washed with brine (2×150 mL), dried over magnesium sulfate, filtered, and concentrated under reduced pressure to afford a colorless oil that crystallized upon standing. The crude was purified by reverse-phase benzyl N-[1-(hydroxymethyl)-3-(1-methylcyclopropyl)propyl]carbamate (4.3 g, 78%) ¹H NMR (400 MHz, CDCl₃) δ 7.44-7.29 (m, 5H), 5.11 (s, 2H), 4.91-4.76 (m, 1H), 3.77-3.51 (m, 3H), 2.14 (br. s, 1H), 1.72-1.55 (m, 1H, overlapped with water), 1.54-1.43 (m, 1H), 1.36-1.24 (m, 2H), 1.00 (s, 3H), 0.24 (s, 4H). ESI-MS m/z calc. 277.1678, found 278.1 (M+1)⁺; Retention time: 3.92 minutes; LC method J.

### Step 2: Benzyl N-[1-(hydroxymethyl)-3-(1-methylcyclopropyl)propyl]carbamate, isomer 1, and benzyl N-[1-(hydroxymethyl)-3-(1-methylcyclopropyl)propyl]carbamate, isomer 2

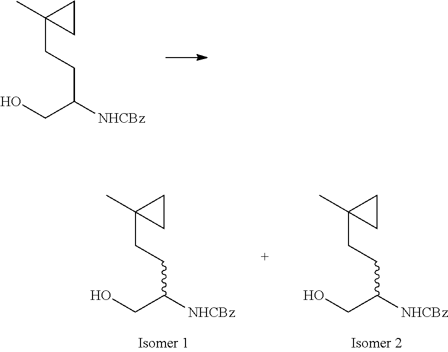

A batch of benzyl N-[1-(hydroxymethyl)-3-(1-methylcyclopropyl)propyl]carbamate (4.3 g, 14.635 mmol) was subjected to a SFC separation using the following conditions: a Cellulose 4 column (250×21.2 mm), 5 M column at 40° C., eluant:7% methanol and 93% CO₂, flow rate: 75 mL/min, concentration: 89.4 mg/mL in methanol (44.7 mg/injection, no modifier), injection volume: 500 μL, pressure: 100 bars, wavelength: 210 nm. Evaporation of the solvents, afforded the two enantiomers as follow: isomer 1, first to elute, benzyl N-[1-(hydroxymethyl)-3-(1-methylcyclopropyl)propyl]carbamate (1.899 g, 92%) as a colorless oil that crystallized upon standing. ¹H NMR (400 MHz, CDCl₃) δ 7.44-7.29 (m, 5H), 5.11 (s, 2H), 4.91-4.76 (m, 1H), 3.77-3.51 (m, 3H), 2.14 (br. s, 1H), 1.72-1.47 (m, 2H, overlapped with water), 1.36-1.24 (m, 2H), 1.00 (s, 3H), 0.24 (s, 4H). ESI-MS m/z calc. 277.1678, found 278.2 (M+1)⁺; Retention time: 3.91 minutes; and Isomer 2, second to elute, benzyl N-[1-(hydroxymethyl)-3-(1-methylcyclopropyl)propyl]carbamate (1.873 g, 90%) as a white solid. ¹H NMR (400 MHz, CDCl₃) δ 7.44-7.29 (m, 5H), 5.11 (s, 2H), 4.91-4.76 (m, 1H), 3.77-3.51 (m, 3H), 2.14 (br. s, 1H), 1.72-1.47 (m, 2H, overlapped with water), 1.36-1.24 (m, 2H), 1.00 (s, 3H), 0.24 (s, 4H). ESI-MS m/z calc. 277.1678, found 278.2 (M+1)⁺; Retention time: 3.92 minutes. LC method J.

### Step 3: 2-Amino-4-(1-methylcyclopropyl)butan-1-ol, isomer 1

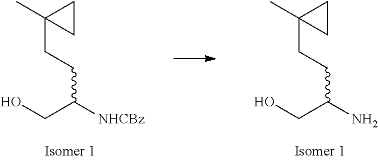

Nitrogen was bubbled to a stirred solution of benzyl N-[1-(hydroxymethyl)-3-(1-methylcyclopropyl)propyl]carbamate (1.885 g, 6.6535 mmol, isomer 1) in methanol (75 mL). Then a slurry of Raney nickel (50 mg, 0.7582 mmol) was added, and nitrogen bubbling was continued for 5 minutes. Hydrogen was then bubbled to the solution for 5 minutes after which time the needle was removed and the reaction was stirred under hydrogen atmosphere. The reaction was then filtered on a pad of celite, washed with methanol (2×15 mL) and concentrated under reduced pressure. The crude was then dissolved in anhydrous methanol (75 mL) and nitrogen was bubbled to the mixture for 15 minutes and Raney nickel (250 mg, 3.7909 mmol) was added. Nitrogen was bubbled again for 15 minutes after which time hydrogen was bubbled for 15 minutes and the reaction was stirred overnight at room temperature under hydrogen atmosphere. The reaction was then filtered on a pad of celite, washed with methanol (2×30 mL) then concentrated under reduced pressure to afford crude 2-amino-4-(1-methylcyclopropyl)butan-1-ol (1.034 g, 88%, isomer 1) as yellow oil that was directly used in the next Step without further purification. ¹H NMR (400 MHz, CDCl₃) δ 3.59 (dd, J=10.3, 3.7 Hz, 1H), 3.26 (dd, J=10.5, 7.8 Hz, 1H), 2.85-2.74 (m, 1H), 1.57-1.46 (m, 1H, overlapped with water), 1.39-1.17 (m, 4H), 1.02 (s, 3H), 0.32-0.18 (m, 4H). (2H missing, labile H). ESI-MS m/z calc. 143.13101, found 144.2 (M+1)⁺; Retention time: 0.9 minutes; LC method I.

### Step 4: 3-[[4-[2-Amino-4-(1-methylcyclopropyl)butoxy]-6-(2,6-dimethylphenyl)pyrimidin-2-yl]sulfamoyl]benzoic acid, isomer 1

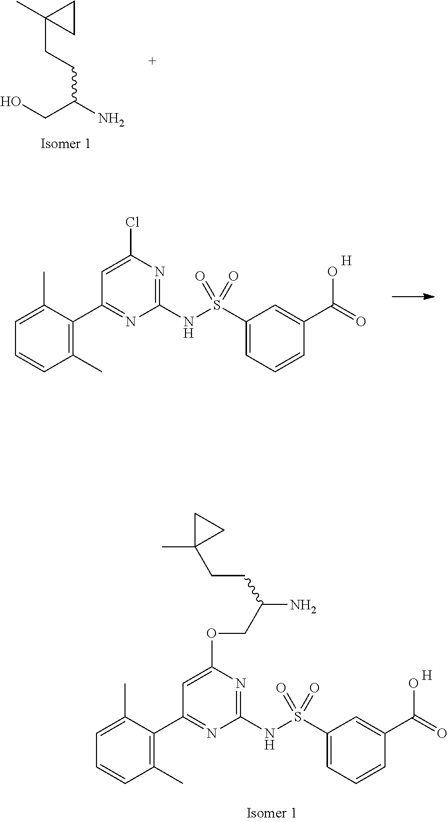

A solution of 2-amino-4-(1-methylcyclopropyl)butan-1-ol isomer 1 (1.034 g, 5.9054 mmol) in DMF (3 mL) was added to a stirred solution of 3-[[4-chloro-6-(2,6-dimethylphenyl)pyrimidin-2-yl]sulfamoyl]benzoic acid, isomer 1(2.45 g, 5.8631 mmol) in 2-methyltetrahydrofuran (25 mL). The resulting mixture was then cooled down to 10-15° C. and sodium tert-butoxide (2.4 g, 24.973 mmol) was added. The reaction was stirred at this temperature for 1 hour after which time an aqueous solution of iN hydrochloric acid (50 mL) was added. The mixture was vigorously stirred for 15 minutes then the layers were separated and the aqueous layer was extracted with 2-methyltetrahydrofuran (5×100 mL). The combined organic layers were washed with brine (300 mL), dried over magnesium sulfate, filtered and concentrated under reduced pressure to afford a pale-yellow foam. The crude foam was purified by reverse-phase chromatography on C₁₈ (column: 275 g; gradient: 5 to 100% methanol in water containing 0.1% v/v of hydrochloric acid; 15CV). The desired fractions were concentrated under reduced pressure and the residue was co-evaporated with acetonitrile (6×10 mL), then freeze-dried to afford 3-[[4-[2-amino-4-(1-methylcyclopropyl)butoxy]-6-(2,6-dimethylphenyl)pyrimidin-2-yl]sulfamoyl]benzoic acid isomer 1 (hydrochloride salt) (2.177 g, 63%) as a pale yellow powder. ¹H NMR (400 MHz, DMSO-d6) δ 13.29 (br. s, 1H), 8.44 (t, J=1.7 Hz, 1H), 8.26 (br. s, 3H), 8.17-8.10 (m, 2H), 7.69 (t, J=7.8 Hz, 1H), 7.29-7.22 (m, 1H), 7.15-7.09 (m, 2H), 6.32 (s, 1H), 4.38 (dd, J=12.0, 2.9 Hz, 1H), 4.27 (dd, J=12.0, 6.4 Hz, 1H), 3.53-3.45 (m, 1H, overlapped with water), 2.00 (s, 6H), 1.78-1.62 (m, 2H), 1.38-1.19 (m, 2H), 0.99 (s, 3H), 0.28-0.19 (m, 4H). (1H missing, labile H). ESI-MS m/z calc. 524.20935, found 525.2 (M+1)⁺; Retention time: 2.53 minutes; LC method J.

### Step 5: 3-[[4-[2-[(6-Cyclopropylfuro[2,3-b]pyrazin-2-yl)methylamino]-4-(1-methylcyclopropyl)butoxy]-6-(2,6-dimethylphenyl)pyrimidin-2-yl]sulfamoyl]benzoic acid, isomer 1

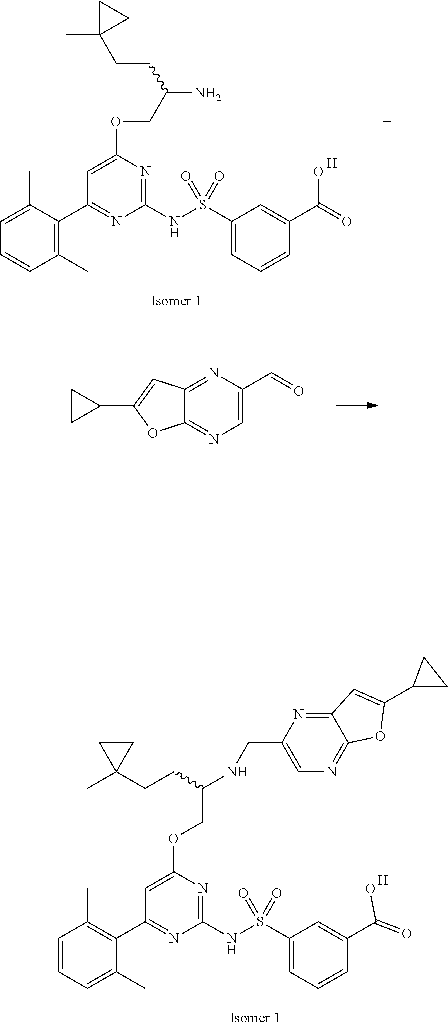

A 4 mL vial was charged under nitrogen with 3-[[4-[2-amino-4-(1-methylcyclopropyl)butoxy]-6-(2,6-dimethylphenyl)pyrimidin-2-yl]sulfamoyl]benzoic acid isomer 1 (hydrochloride salt) (70 mg, 0.1248 mmol) (isomer 1), 6-cyclopropylfuro[2,3-b]pyrazine-2-carbaldehyde (23.7 mg, 0.1259 mmol), anhydrous DCM (0.9 mL), and acetic acid (0.008 mL, 0.1407 mmol). The mixture was cooled down in an ice bath. DIEA (0.03 mL, 0.1722 mmol) was added, followed by sodium triacetoxyborohydride (Sodium salt) (137.1 mg, 0.6469 mmol) and the reaction was vigorously stirred at 0° C. for 1 h. The reaction was quenched with 3 N aqueous HCl, diluted with MeOH and DMSO, and the resulting solution was filtered, and purified by reverse phase HPLC (1-99% acetonitrile/5 mM aqueous HCl over 15 min) to give 3-[[4-[2-[(6-cyclopropylfuro[2,3-b]pyrazin-2-yl)methylamino]-4-(1-methylcyclopropyl)butoxy]-6-(2,6-dimethylphenyl)pyrimidin-2-yl]sulfamoyl]benzoic acid isomer 1(hydrochloride salt) (76.3 mg, 72%) as a white solid. ESI-MS m/z calc. 696.273, found 697.8 (M+1)⁺; Retention time: 1.48 minutes; LC method A.

### Step 6: 12-[(6-Cyclopropylfuro[2,3-b]pyrazin-2-yl)methyl]-6-(2,6-dimethylphenyl)-11-[2-(1-methylcyclopropyl)ethyl]-2,2-dioxo-9-oxa-2λ⁶-thia-3,5,12,19-tetrazatricyclo[12.3.1.14,8]nonadeca-1(18),4(19),5,7,4,16-hexaen-13-one, isomer 1 (Compound I-71

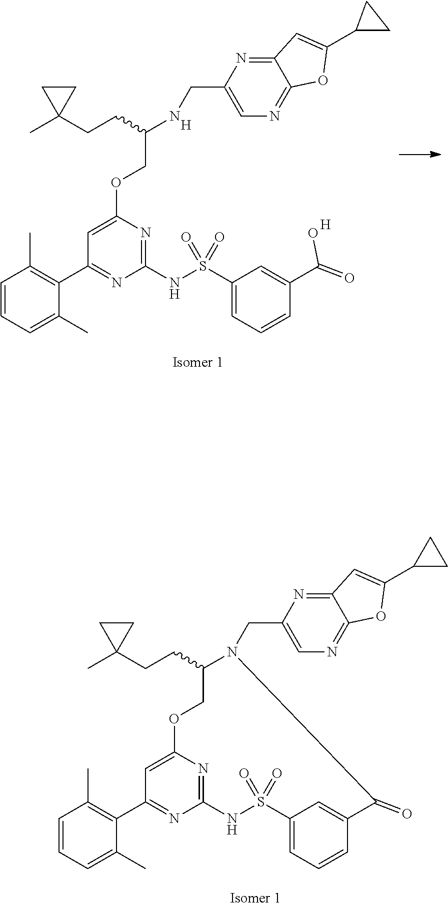

3-[[4-[2-[(6-cyclopropylfuro[2,3-b]pyrazin-2-yl)methylamino]-4-(1-methylcyclopropyl)butoxy]-6-(2,6-dimethylphenyl)pyrimidin-2-yl]sulfamoyl]benzoic acid isomer 1(hydrochloride salt) (76.3 mg, 0.08949 mmol) was combined with CDMT (53.3 mg, 0.3036 mmol) in DMF (7 mL) and cooled to 0° C. N-methylmorpholine (0.05 mL, 0.4548 mmol) was added by syringe and the reaction was stirred at 0° C. for 30 minutes. The ice bath was then removed and stirring was continued for an additional 24 hours at room temperature. The reaction mixture was then partitioned between 50 mL 1 M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give 12-[(6-cyclopropylfuro[2,3-b]pyrazin-2-yl)methyl]-6-(2,6-dimethylphenyl)-11-[2-(1-methylcyclopropyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one isomer 1 (29.4 mg, 48%) as a white solid. ¹H NMR (400 MHz, CDCl₃) δ 8.62 (t, J=1.8 Hz, 1H), 8.36 (s, 1H), 8.11 (d, J=7.9 Hz, 1H), 7.79 (dt, J=7.8, 1.3 Hz, 1H), 7.64 (t, J=7.8 Hz, 1H), 7.19 (t, J=7.6 Hz, 1H), 7.00 (d, J=7.6 Hz, 2H), 6.61 (s, 1H), 6.16 (s, 1H), 5.38-5.27 (m, 2H), 4.31-4.20 (m, 2H), 4.08-3.97 (m, 1H), 2.18-2.09 (m, 1H), 1.96 (s, 6H), 1.86-1.68 (m, 2H), 1.23-1.09 (m, 5H), 0.94-0.83 (m, 4H), 0.21-0.09 (m, 4H). ESI-MS m/z calc. 678.26245, found 679.4 (M+1)⁺; Retention time: 2.15 minutes; LC method A.

### Example 57: Preparation of Compound I-87

### Step 1: 3-Fluoro-6-(methylamino)pyridine-2-carboxylic acid

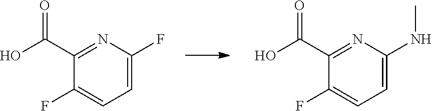

To two separate microwave vials containing 3,6-difluoropyridine-2-carboxylic acid (5 g, 31.429 mmol) (2.5 g/vial) was added MeNH₂ in water (40 mL of 40% w/v, 515.18 mmol) (20 mL/vial) and the reaction was heated at 100° C. for 11.5 h in a microwave reactor. The reaction was concentrated in vacuo to a yellow oil to which was added aqueous HCl (40 mL of 1 M, 40.000 mmol) and Et₂O (20 mL) and the resulting precipitate was collected by vacuum filtration. The filtrate was rinsed with Et₂O (2×20 mL) then dried under high vacuum to provide 3-fluoro-6-(methylamino)pyridine-2-carboxylic acid (hydrochloride salt) (5 g, 73%) as a canary yellow solid. ESI-MS m/z calc. 170.04915, found 171.5 (M+1)⁺; Retention time: 0.78 minutes; LC method D.

### Step 2: 3-Fluoro-5-iodo-6-(methylamino)pyridine-2-carboxylic acid

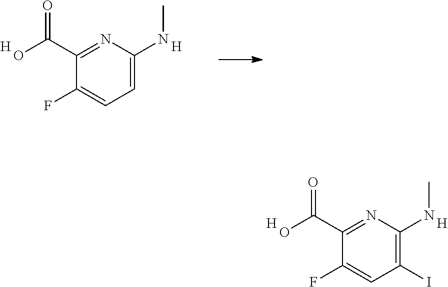

To a suspension of 3-fluoro-6-(methylamino)pyridine-2-carboxylic acid (100 mg, 0.5877 mmol) in AcOH (2.5 mL) was added NIS (145 mg, 0.6445 mmol) at room temperature and the reaction was stirred for 2 h. Then DMSO (0.8 mL) was added and the reaction was heated at 55° C. for 16 h. The reaction was cooled down to room temperature, diluted with EtOAc (20 mL), washed with water (10 mL), aqueous 20% w/v sodium metabisulfite (15 mL), then the organics were extracted with 2 M NaOH (2×3 mL) and then the aqueous phase was acidified with 6 M HCl to pH=3. The resulting solids were collected by filtration to provide 3-fluoro-5-iodo-6-(methylamino)pyridine-2-carboxylic acid (80 mg, 46%) as a grey solid. ¹H NMR (500 MHz, DMSO-d6) δ 8.08 (d, J=9.0 Hz, 1H), 6.20 (q, J=4.5, 4.5, 4.5 Hz, 1H), 2.81 (d, J=4.6 Hz, 3H). ESI-MS m/z calc. 295.9458, found 297.1 (M+1)⁺; Retention time: 2.6 minutes; LC method D.

### Step 3: 3-Fluoro-6-(methylamino)-5-(3-methylbut-1-ynyl)pyridine-2-carboxylic acid

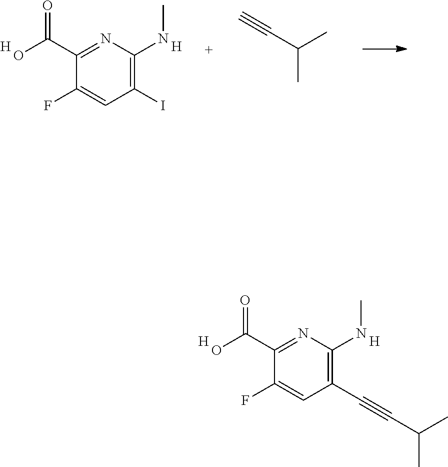

Under argon, a solution of 3-fluoro-5-iodo-6-(methylamino)pyridine-2-carboxylic acid (40 mg, 0.1351 mmol) in degassed TEA (1.40 mL) and degassed THE (0.250 mL) was cooled to 0° C. and 3-methylbut-1-yne (66.000 mg, 0.100 mL, 0.9689 mmol) was added, followed by CuI (1.0856 mg, 0.0057 mmol) and Pd(PPh₃)₂Cl₂ (6.8 mg, 0.0097 mmol). The reaction was slowly allowed to warm to room temperature over 4 h. Additional 3-methylbut-1-yne (66.000 mg, 0.100 mL, 0.9689 mmol) and CuI (1 mg, 0.0053 mmol) were added under argon and the reaction was stirred for 12 h. 3 M HCl was added (until pH=4) and the aqueous phase was extracted with EtOAc (3×20 mL). The combined organic extracts were mixed with 2M NaOH. The aqueous phase was separated then acidified with 3 M HCl (until pH=4) and the resulting suspension was extracted with EtOAc (3×20 mL), dried over MgSO₄, filtered and concentrated in vacuo to provide 3-fluoro-6-(methylamino)-5-(3-methylbut-1-ynyl)pyridine-2-carboxylic acid (30 mg, 89%) as a pale orange solid. ¹H NMR (500 MHz, Chloroform-d) δ 7.38 (d, J=8.9 Hz, 1H), 5.30 (s, 1H), 3.03 (s, 3H), 2.92-2.81 (m, 1H), 1.35-1.27 (m, 6H). ESI-MS m/z calc. 236.0961, found 237.2 (M+1)⁺; Retention time: 3.99 minutes; LC method D.

### Step 4: 5-Fluoro-2-isopropyl-1-methyl-pyrrolo[2,3-b]pyridine-6-carboxylic acid

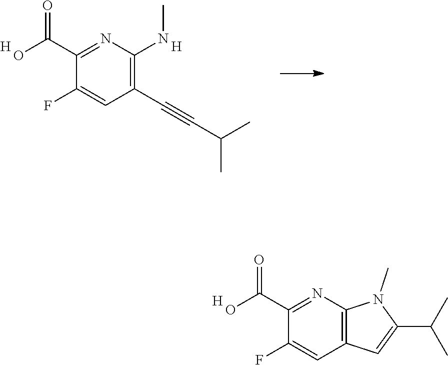

To a solution of t-BuOK (40.6 mg, 0.3618 mmol) in NMP (0.300 mL) was added 3-fluoro-6-(methylamino)-5-(3-methylbut-1-ynyl)pyridine-2-carboxylic acid (30 mg, 0.1206 mmol) in NMP (0.500 mL) and the reaction was stirred for 3 h then heated to 80° C. The mixture was quenched with 1 M HCl (2 mL) and extracted with EtOAc (3×15 mL), dried over MgSO₄, filtered and concentrated in vacuo. Brine (15 mL) was added and the aqueous phase was extracted with a 5:1 mixture of Et₂O-Hexanes (3×10 mL) and the organics were combined and dried over MgSO₄, filtered and concentrated in vacuo to provide 5-fluoro-2-isopropyl-1-methyl-pyrrolo[2,3-b]pyridine-6-carboxylic acid (11 mg, 38%) as an off white solid. ¹H NMR (500 MHz, Chloroform-d) δ 11.06-10.33 (m, 1H), 7.65 (d, J=10.7 Hz, 1H), 6.33 (d, J=0.9 Hz, 1H), 3.84 (s, 3H), 3.19-3.09 (m, 1H), 1.41-1.37 (m, 6H). ESI-MS m/z calc. 236.0961, found 237.5 (M+1)⁺; Retention time: 4.01 minutes; LC method D.

### Step 5: 5-Fluoro-2-isopropyl-1-methyl-pyrrolo[2,3-b]pyridine-6-carboxylic acid

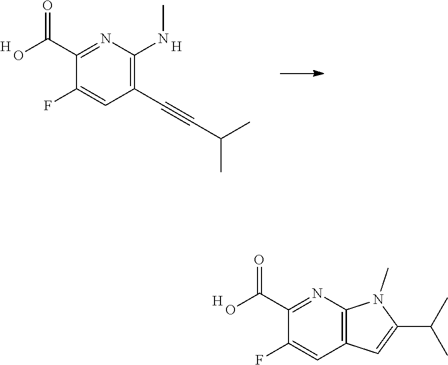

To a solution of tBuOK (40.6 mg, 0.3618 mmol) in NMP (0.300 mL) was added 3-fluoro-6-(methylamino)-5-(3-methylbut-1-ynyl)pyridine-2-carboxylic acid (30 mg, 0.1206 mmol) in NMP (0.500 mL) and the reaction was stirred for 3 h, then heated to 80° C. After completion, the mixture was quenched with 1 M HCl (2 mL) and extracted with EtOAc (3×15 mL), dried over MgSO₄, filtered, and concentrated in vacuo. Brine (15 mL) was added and the aqeuous phase was extracted with a 5:1 mixture of diethyl ether-hexanes (3×10 mL). The organics were combined and dried over MgSO₄, filtered and concentrated in vacuo to provide 5-fluoro-2-isopropyl-1-methyl-pyrrolo[2,3-b]pyridine-6-carboxylic acid (11 mg, 38%) as an off white solid. ¹H NMR (500 MHz, Chloroform-d) δ 11.06-10.33 (m, 1H), 7.65 (d, J=10.7 Hz, 1H), 6.33 (d, J=0.9 Hz, 1H), 3.84 (s, 3H), 3.19-3.09 (m, 1H), 1.41-1.37 (m, 6H). ESI-MS m/z calc. 236.0961, found 237.5 (M+1)⁺; Retention time: 4.01 minutes; LC method D.

### Step 6: Methyl 5-fluoro-2-isopropyl-1-methyl-pyrrolo[2,3-b]pyridine-6-carboxylate

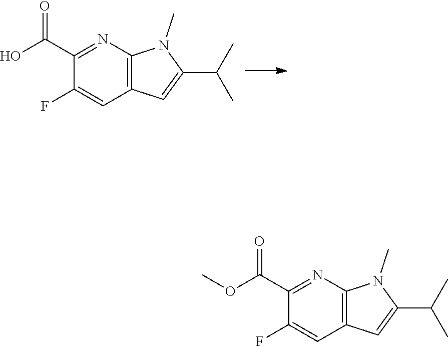 [SMILES: CC(C)c1cc2cc(F)c(C(=O)O)nc2n1C ; COC(=O)c1nc2c(cc1F)cc(C(C)C)n2C]

To a solution of 5-fluoro-2-isopropyl-1-methyl-pyrrolo[2,3-b]pyridine-6-carboxylic acid (170 mg, 0.7124 mmol) in DMF (2.65 mL) was added potassium carbonate (231 mg, 1.6714 mmol) and Mel (241.68 mg, 0.106 mL, 1.7027 mmol) at room temperature and the mixture was stirred for 4 h. The reaction was quenched with saturated aqueous ammonium chloride (10 mL), and the aqueous phase was extracted with EtOAc (4×30 mL), washed with brine (2×100 mL), dried over sodium sulfate filtered and concentrated in vacuo to provide methyl 5-fluoro-2-isopropyl-1-methyl-pyrrolo[2,3-b]pyridine-6-carboxylate (177 mg, 99%) as a pale green oil. ¹H NMR (500 MHz, Chloroform-d) δ 7.55 (d, J=11.1 Hz, 1H), 6.25 (s, 1H), 4.01 (s, 3H), 3.87 (s, 3H), 3.18-3.08 (m, 1H), 1.36 (d, J=6.8 Hz, 6H). ESI-MS m/z calc. 250.11176, found 251.2 (M+1)⁺; Retention time: 4.7 minutes; LC method D.

### Step 7: 5-Fluoro-2-isopropyl-1-methyl-pyrrolo[2,3-b]pyridine-6-carbaldehyde

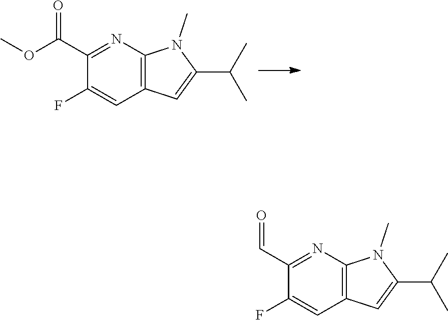 [SMILES: COC(=O)c1nc2c(cc1F)cc(C(C)C)n2C ; CC(C)c1cc2cc(F)c(C=O)nc2n1C]

A solution of methyl 5-fluoro-2-isopropyl-1-methyl-pyrrolo[2,3-b]pyridine-6-carboxylate (177 mg, 0.7072 mmol) in DCM (3 mL) was cooled in a −78° C. bath for 15 minutes, then DIBAL in DCM (1.10 mL of 1 M, 1.1000 mmol) was added dropwise and the reaction was stirred for 40 minutes at −78° C. Additional DIBAL in DCM (0.200 mL of 1 M, 0.2000 mmol) was added. The reaction was quenched with MeOH-H₂O (1:1; 3 mL) at −78° C. and warmed to room temperature, then stirred for 1 h at room temperature. The reaction was concentrated in vacuo, diluted with DCM (15 mL) washed with 1 M HCl (15 mL) and the aqueous layer was extracted with DCM (2×15 mL), the organics were combined and washed with brine (20 mL), dried over MgSO₄, filtered, and concentrated in vacuo. The residue was purified by flash chromatography (loaded onto a 12 g silica cartridge with benzene, and eluted with 0-20% diethyl ether in hexanes over a 30 min gradient) to provide 5-fluoro-2-isopropyl-1-methyl-pyrrolo[2,3-b]pyridine-6-carbaldehyde (96.4 mg, 59%) as a pale yellow solid. ¹H NMR (500 MHz, Chloroform-d) δ 10.27 (s, 1H), 7.54 (d, J=11.0 Hz, 1H), 6.30 (d, J=0.7 Hz, 1H), 3.89 (s, 3H), 3.22-3.10 (m, 1H), 1.38 (d, J=6.8 Hz, 6H). ESI-MS m/z calc. 220.1012, found 221.2 (M+1)⁺; Retention time: 2.3 minutes; LC method H.

### Step 8: 3-[[4-[(2R)-3-(1-Bicyclo[1.1.1]pentanyl)-2-[(5-fluoro-2-isopropyl-1-methyl-pyrrolo[2,3-b]pyridin-6-yl)methylamino]propoxy]-6-(2,6-dimethylphenyl)pyrimidin-2-yl]sulfamoyl]benzoic acid

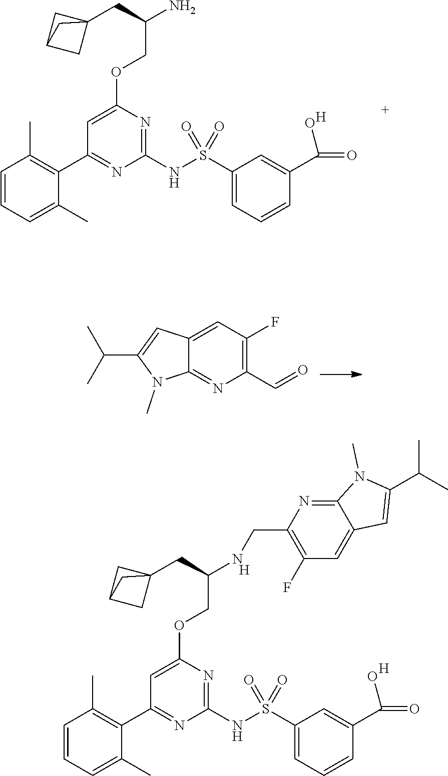

A 4 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (50 mg, 0.08943 mmol), 5-fluoro-2-isopropyl-1-methyl-pyrrolo[2,3-b]pyridine-6-carbaldehyde (19.8 mg, 0.08990 mmol), anhydrous DCM (0.7 mL), and acetic acid (0.006 mL, 0.1055 mmol). The mixture was cooled down in an ice bath. DIEA (0.04 mL, 0.2296 mmol) was added, followed by sodium triacetoxyborohydride (194.1 mg, 0.9158 mmol), and the reaction was vigorously stirred at 0° C. for 2 h. The reaction was quenched with 3 N aqueous HCl, diluted with MeOH and DMSO, and the resulting solution was filtered, and purified by reverse phase HPLC (1-99% acetonitrile/5 mM aqueous HCl over 15 min) to give 3-[[4-[(2R)-3-(1-bicyclo[1.1.1]pentanyl)-2-[(5-fluoro-2-isopropyl-1-methyl-pyrrolo[2,3-b]pyridin-6-yl)methylamino]propoxy]-6-(2,6-dimethylphenyl)pyrimidin-2-yl]sulfamoyl]benzoic acid (hydrochloride salt) (47.1 mg, 65%) as a white solid. ESI-MS m/z calc. 726.3, found 727.7 (M+1)⁺; Retention time: 1.71 minutes. ESI-MS m/z calc. 726.3, found 727.7 (M+1)⁺; Retention time: 1.71 minutes; LC method A.

### Step 9: (11R)-11-(1-Bicyclo[1.1.1]pentanylmethyl)-6-(2,6-dimethylphenyl)-12-[(5-fluoro-2-isopropyl-1-methyl-pyrrolo[2,3-b]pyridin-6-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one (Compound I-87

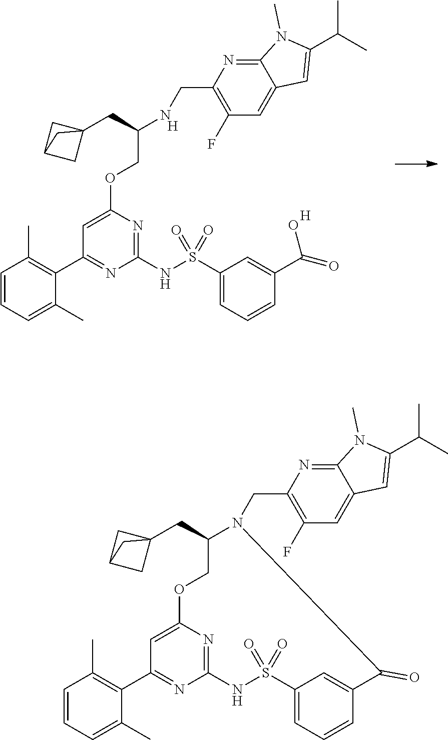

3-[[4-[(2R)-3-(1-Bicyclo[1.1.1]pentanyl)-2-[(5-fluoro-2-isopropyl-1-methyl-pyrrolo[2,3-b]pyridin-6-yl)methylamino]propoxy]-6-(2,6-dimethylphenyl)pyrimidin-2-yl]sulfamoyl]benzoic acid (hydrochloride salt) (47.1 mg, 0.05800 mmol) was combined with CDMT (31.4 mg, 0.1788 mmol) in DMF (5.4 mL) and cooled to 0° C. N-methylmorpholine (0.04 mL, 0.3638 mmol) was added by syringe and the reaction was stirred at 0° C. for 30 minutes. The ice bath was then removed and stirring was continued for an additional 24 hours at room temperature. The reaction mixture was then partitioned between 50 mL 1 M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-11-(1-bicyclo[1.1.1]pentanylmethyl)-6-(2,6-dimethylphenyl)-12-[(5-fluoro-2-isopropyl-1-methyl-pyrrolo[2,3-b]pyridin-6-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (16.2 mg, 39%) as a white solid. ¹H NMR (400 MHz, CDCl₃) δ 9.18 (t, J=1.8 Hz, 1H), 8.04 (d, J=7.9 Hz, 1H), 7.93 (dt, J=7.7, 1.3 Hz, 1H), 7.67 (t, J=7.8 Hz, 1H), 7.47 (d, J=10.3 Hz, 1H), 7.19 (t, J=7.6 Hz, 1H), 7.03 (d, J=7.6 Hz, 2H), 6.14 (s, 2H), 5.62-5.51 (m, 2H), 4.35-4.28 (m, 1H), 4.11-4.03 (m, 1H), 3.88 (s, 3H), 3.68 (t, J=11.4 Hz, 1H), 3.08 (hept, J=6.7 Hz, 1H), 2.46 (s, 1H), 2.00 (s, 6H), 1.98-1.91 (m, 1H), 1.78 (dd, J=15.4, 3.4 Hz, 1H), 1.66-1.59 (m, 6H), 1.34 (d, J=6.8 Hz, 3H), 1.30 (d, J=6.8 Hz, 3H). 19F NMR (376 MHz, CDCl₃) δ −140.51. ¹⁹F NMR (376 MHz, CDCl₃) δ −140.51. ESI-MS m/z calc. 708.2894, found 709.4 (M+1)⁺; Retention time: 2.49 minutes; LC method A.

### Example 58: Preparation of Compound I-88

### Step 1: 5-Bromo-N3-methyl-pyrazine-2,3-diamine

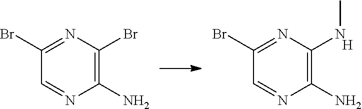

A suspension of 3,5-dibromopyrazin-2-amine (1 g, 3.9542 mmol) in MeNH₂ in water (10 mL of 40% w/v, 128.80 mmol) was heated in a microwave reactor at 130° C. for 1 h. The reaction was cooled to room temperature and extracted with DCM (3×15 mL), dried over MgSO₄, filtered and concentrated in vacuo to provide 5-bromo-N3-methyl-pyrazine-2,3-diamine (802 mg, 100%) as a tan solid. ¹H NMR (400 MHz, Chloroform-d) δ 7.43 (s, 1H), 4.48 (s, 1H), 4.24 (s, 2H), 2.99 (d, J=5.0 Hz, 3H). ESI-MS m/z calc. 201.98541, found 203.1 (M+1)⁺; Retention time: 2.4 minutes; LC method D.

### Step 2: 5-Bromo-2-isopropyl-3-methyl-imidazo[4,5-b]pyrazine

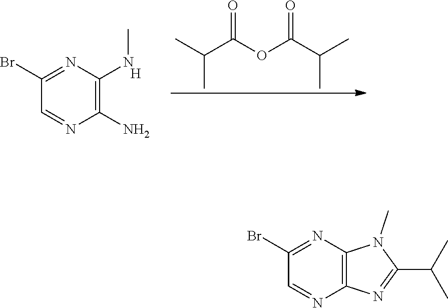

To a solution of 2-methylpropanoyl 2-methylpropanoate (248.04 mg, 0.260 mL, 1.5679 mmol) in isobutyric acid (2 mL) was added 5-bromo-N3-methyl-pyrazine-2,3-diamine (300 mg, 1.4775 mmol) and the reaction was stirred at room temperature for 10 minutes then heated in a microwave reactor at 130° C. for 5 h and 20 min. To the reaction was added 2-methylpropanoyl 2-methylpropanoate (477.00 mg, 0.500 mL, 3.0153 mmol) and it was heated at 130° C. for 2 h. The reaction was cooled to room temperature and diluted with DCM (30 mL) and the organic phase was washed with 1M NaOH (30 mL). The aqueous phase was back extracted with DCM (3×25 mL) and filtered through filter paper to remove emulsions. The organic phase was washed with brine, dried over MgSO₄, filtered and concentrated in vacuo. The residue was purified by flash chromatography (loaded onto a 12 g silica cartridge with benzene, and eluted with 0-40% EtOAc in hexanes over a 30 min gradient) to provide 5-bromo-2-isopropyl-3-methyl-imidazo[4,5-b]pyrazine (270 mg, 72%) as an off white solid. ¹H NMR (500 MHz, Chloroform-d) δ 8.50 (s, 1H), 3.83 (s, 3H), 3.25 (hept, J=6.8 Hz, 1H), 1.47 (d, J=6.8 Hz, 6H). ¹³C NMR (126 MHz, CDCl₃) δ 165.38, 147.56, 141.09, 132.60, 28.70, 27.58, 20.77. ESI-MS m/z calc. 254.01671, found 255.4 (M+1)⁺; Retention time: 2.87 minutes; LC method D.

### Step 3: Methyl 2-isopropyl-3-methyl-imidazo[4,5-b]pyrazine-5-carboxylate

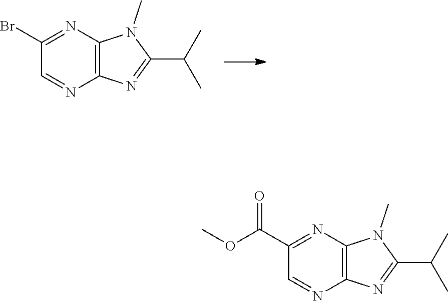 [SMILES: CC(C)c1nc2ncc(Br)nc2n1C ; COC(=O)c1cnc2nc(C(C)C)n(C)c2n1]

To a solution of 5-bromo-2-isopropyl-1-methyl-imidazo[4,5-b]pyrazine (4.03 g, 15.797 mmol) in MeOH (80 mL) was added Pd(dppf)Cl₂-DCM complex (1.25 g, 1.5307 mmol) then TEA (32.670 g, 45 mL, 322.86 mmol) was added. The vessel was sealed and pressurized with CO gas (100 psi) and vented 3 times, then re-pressurized at 100 psi and heated at 80° C. for 30 minutes. The reaction was cooled to room temperature and stirred at 100 psi for 12 h. Then the pressure was released, and the mixture was concentrated in vacuo. The reaction was diluted with toluene and the TEA salts were filtered off through a pad of celite. The supernatant was concentrated in vacuo and the residue was purified by flash chromatography (loaded with toluene onto a 120 g silica cartridge pre-equilibrated with 1% TEA in hexanes, and eluted with 0-50% EtOAc in hexanes over a 60 min gradient and held at 47% EtOAc for 15 min) to provide methyl 2-isopropyl-1-methyl-imidazo[4,5-b]pyrazine-5-carboxylate (2.8 g, 76%) as a colorless solid. ESI-MS m/z calc. 234.11168, found 235.3 (M+1)⁺; Retention time: 2.18 minutes; LC method D.

### Step 4: 2-Isopropyl-3-methyl-imidazo[4,5-b]pyrazine-5-carbaldehyde

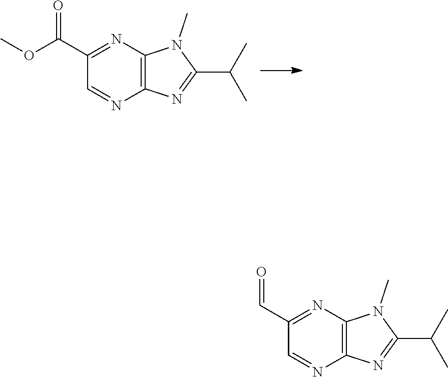 [SMILES: COC(=O)c1cnc2nc(C(C)C)n(C)c2n1 ; CC(C)c1nc2ncc(C=O)nc2n1C]

A solution of methyl 2-isopropyl-3-methyl-imidazo[4,5-b]pyrazine-5-carboxylate (2.7 g, 11.526 mmol) in DCM (40 mL) was cooled in bath at −78° C. for 15 minutes then DIBAL in DCM (17.551 mL of 1 M, 17.551 mmol) was added dropwise. The reaction was stirred at this temperature for 25 min at −78° C. The reaction was quenched with MeOH—H₂O (1:1, 20 mL) at −78° C. and stirred 10 minutes then warmed to room temperature and further stirred for 30 min. The reaction was concentrated in vacuo and DCM (150 mL) and 1 M HCl (50 mL) were added. The layers were separated and the aqueous phase was extracted with DCM (3×15 mL). The combined organics were washed with brine (50 mL), dried over MgSO₄, filtered, and concentrated in vacuo. The residue was purified by flash chromatography (dry loaded with DCM onto 50 g silica and attached to an 80 g silica cartridge and eluted with 0-50% EtOAc in hexanes over a 30 min gradient) to provide 2-isopropyl-3-methyl-imidazo[4,5-b]pyrazine-5-carbaldehyde (972 mg, 41%) as a white crystalline solid. ¹H NMR (500 MHz, Chloroform-d) δ 10.19 (s, 1H), 9.14 (s, 1H), 3.94 (s, 3H), 3.35 (hept, J=6.8 Hz, 1H), 1.52 (d, J=6.8 Hz, 6H). ESI-MS m/z calc. 204.1011, found 205.0 (M+1)⁺; Retention time: 1.11 minutes; LC method H.

### Step 5: 3-[[4-[(2R)-3-(1-Bicyclo[1.1.1]pentanyl)-2-[(2-isopropyl-3-methyl-imidazo[4,5-b]pyrazin-5-yl)methylamino]propoxy]-6-(2,6-dimethylphenyl)pyrimidin-2-yl]sulfamoyl]benzoic acid

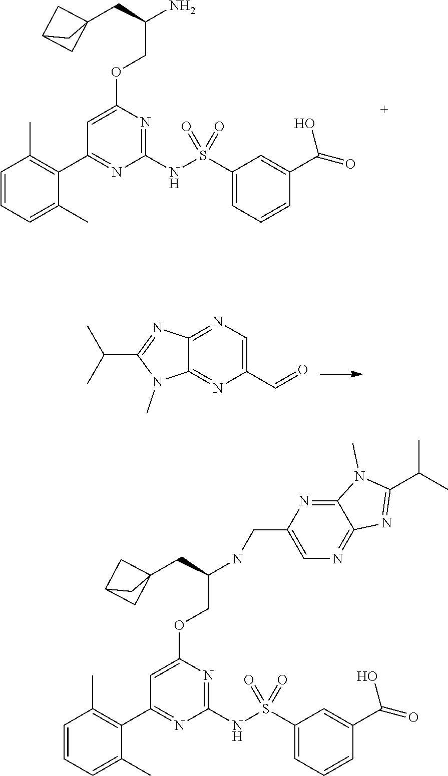 [SMILES: Cc1cccc(C)c1-c1cc(OC[C@H](N)CC23CC(C2)C3)nc(NS(=O)(=O)c2cccc(C(=O)O)c2)n1 ; CC(C)c1nc2ncc(C=O)nc2n1C ; Cc1cccc(C)c1-c1cc(OC[C@@H](CC23CC(C2)C3)NCc2cnc3nc(C(C)C)n(C)c3n2)nc(NS(=O)(=O)c2cccc(C(=O)O)c2)n1]

A 4 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (70 mg, 0.1252 mmol), 2-isopropyl-3-methyl-imidazo[4,5-b]pyrazine-5-carbaldehyde (25.7 mg, 0.1258 mmol), anhydrous DCM (1.0 mL), and acetic acid (0.008 mL, 0.1407 mmol). The mixture was cooled down in an ice bath. DIEA (0.06 mL, 0.3445 mmol) was added, followed by sodium triacetoxyborohydride (278.5 mg, 1.314 mmol), and the reaction was vigorously stirred at 0° C. for 3 h. The reaction was quenched with 3 N aqueous HCl, diluted with MeOH and DMSO, and the resulting solution was filtered, and purified by reverse phase HPLC (1-99% acetonitrile/5 mM aqueous HCl over 15 min) to give 3-[[4-[(2R)-3-(1-bicyclo[1.1.1]pentanyl)-2-[(2-isopropyl-3-methyl-imidazo[4,5-b]pyrazin-5-yl)methylamino]propoxy]-6-(2,6-dimethylphenyl)pyrimidin-2-yl]sulfamoyl]benzoic acid (hydrochloride salt) (70.5 mg, 75%) as a white solid. ESI-MS m/z calc. 710.29987, found 711.4 (M+1)⁺; Retention time: 1.31 minutes; LC method A.

### Step 6: (11R)-11-(1-Bicyclo[1.1.1]pentanylmethyl)-6-(2,6-dimethylphenyl)-12-[(2-isopropyl-3-methyl-imidazo[4,5-b]pyrazin-5-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one (Compound I-88

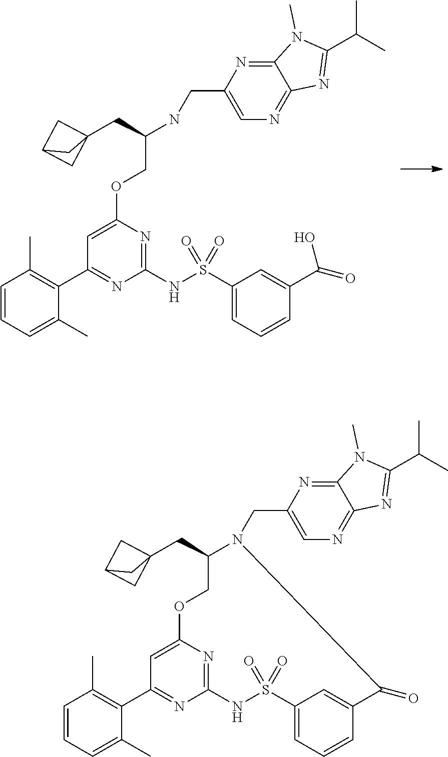 [SMILES: Cc1cccc(C)c1-c1cc(OC[C@@H](CC23CC(C2)C3)NCc2cnc3nc(C(C)C)n(C)c3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3nc(C(C)C)n(C)c3n1)[C@H](CC13CC(C1)C3)CO2]

3-[[4-[(2R)-3-(1-Bicyclo[1.1.1]pentanyl)-2-[(2-isopropyl-3-methyl-imidazo[4,5-b]pyrazin-5-yl)methylamino]propoxy]-6-(2,6-dimethylphenyl)pyrimidin-2-yl]sulfamoyl]benzoic acid (hydrochloride salt) (70.5 mg, 0.09434 mmol) was combined with CDMT (53.5 mg, 0.3047 mmol) in DMF (8.6 mL) and cooled to 0° C. N-methylmorpholine (0.062 mL, 0.5639 mmol) was added by syringe and the reaction was stirred at 0° C. for 30 minutes. The ice bath was then removed and stirring was continued for an additional 16 hours at room temperature. The reaction mixture was then partitioned between 50 mL 1 M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-11-(1-bicyclo[1.1.1]pentanylmethyl)-6-(2,6-dimethylphenyl)-12-[(2-isopropyl-3-methyl-imidazo[4,5-b]pyrazin-5-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (43.4 mg, 66%). ¹H NMR (400 MHz, CDCl₃) δ 8.99 (t, J=1.8 Hz, 1H), 8.60 (s, 1H), 8.01 (d, J=7.9 Hz, 1H), 7.88 (dt, J=7.7, 1.4 Hz, 1H), 7.65 (t, J=7.8 Hz, 1H), 7.22 (t, J=7.6 Hz, 1H), 7.04 (d, J=7.6 Hz, 2H), 6.17 (s, 1H), 5.58 (dd, J=11.0, 3.7 Hz, 1H), 5.47 (d, J=16.2 Hz, 1H), 4.34 (d, J=16.2 Hz, 1H), 4.08-3.99 (m, 1H), 3.95 (s, 3H), 3.89 (t, J=11.3 Hz, 1H), 3.33 (hept, J=6.9 Hz, 1H), 2.47 (s, 1H), 2.03 (s, 6H), 2.00-1.90 (m, 1H), 1.82 (dd, J=15.5, 3.5 Hz, 1H), 1.67-1.58 (m, 6H), 1.53 (d, J=6.8 Hz, 3H), 1.50 (d, J=6.8 Hz, 3H). ESI-MS m/z calc. 692.2893, found 693.4 (M+1)⁺; Retention time: 1.78 minutes; LC method A.

### Example 59: Preparation of Compound I-96

### Step 1: 5-Chloro-1-[(4-methoxyphenyl)methyl]-3-[2-(1-methylcyclopropyl)ethynyl]pyrazin-2-one

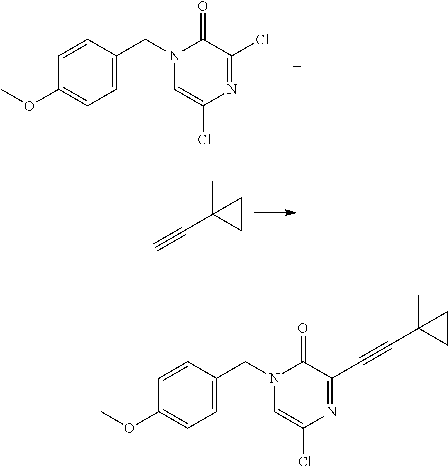

To a 20 mL microwave vial was charged 3,5-dichloro-1-[(4-methoxyphenyl)methyl]pyrazin-2-one (350 mg, 1.228 mmol), Bis(triphenylphosphine) palladium(II) dichloride (16 mg, 0.02280 mmol), CuI (10 mg, 0.05251 mmol) in DMF (2.5 mL) and TEA (1.0 mL, 7.175 mmol). Then, 1-ethynyl-1-methyl-cyclopropane (100 mg, 1.248 mmol) was added, and the vessel was sealed. The mixture was irradiated at 80° C. μW for 10 min. The reaction mixture was allowed to cool, diluted with DCM (20 mL) and washed with water (2×10 mL). The organic layer was washed with brine (10 mL), dried with sodium sulfate, filtered and concentrated under vacuo. The resulting material was purified by silica gel chromatography (40 g of silica) using a gradient eluent of 100% hexanes to 80% ethyl acetate in hexanes to afford as a yellow oil 5-chloro-1-[(4-methoxyphenyl)methyl]-3-[2-(1-methylcyclopropyl)ethynyl]pyrazin-2-one (287 mg, 71%), ¹H NMR (400 MHz, DMSO-d6) δ 8.15 (s, 1H), 7.38-7.30 (m, 2H), 6.95-6.87 (m, 2H), 4.96 (s, 2H), 3.73 (s, 3H), 1.31 (s, 3H), 1.04-0.96 (m, 2H), 0.88-0.80 (m, 2H). ESI-MS m/z calc. 328.09787, found 329.2 (M+1)⁺; Retention time: 1.83 minutes; LC method A.

### Step 2: 2-Chloro-6-(1-methylcyclopropyl)furo[2,3-b]pyrazine

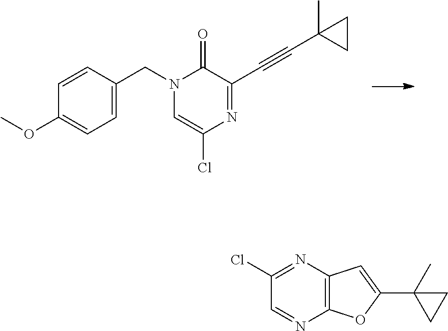 [SMILES: COc1ccc(Cn2cc(Cl)nc(C#CC3(C)CC3)c2=O)cc1 ; CC1(c2cc3nc(Cl)cnc3o2)CC1]

To a solution of 5-chloro-1-[(4-methoxyphenyl)methyl]-3-[2-(1-methylcyclopropyl)ethynyl]pyrazin-2-one (283 mg, 0.8607 mmol) in DCM (5.0 mL), Trifluoromethanesulfonate (6 mg, 0.02335 mmol) (silver salt) and TFA (600 μL, 7.788 mmol) were added and stirred at room temperature for 90 minutes. The residue was concentrated in vacuo and purified by silica gel chromatography (40 g of silica) using a gradient eluent of 100% hexanes to 30% ethyl acetate in hexanes to give as a pale yellow solid 2-chloro-6-(1-methylcyclopropyl)furo[2,3-b]pyrazine (148.0 mg, 82%), ¹H NMR (400 MHz, Methanol-d4) δ 8.14 (s, 1H), 6.73 (s, 1H), 1.55 (s, 3H), 1.41-1.34 (m, 2H), 1.07-0.99 (m, 2H). ESI-MS m z calc. 208.04034, found 209.2 (M+1)⁺; Retention time: 1.76 minutes; LC method A.

### Step 3: Methyl 6-(1-methylcyclopropyl)furo[2,3-b]pyrazine-2-carboxylate

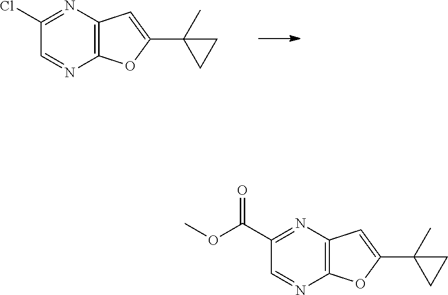 [SMILES: CC1(c2cc3nc(Cl)cnc3o2)CC1 ; COC(=O)c1cnc2oc(C3(C)CC3)cc2n1]

A mixture of 2-chloro-6-(1-methylcyclopropyl)furo[2,3-b]pyrazine (144 mg, 0.6902 mmol) and Pd(dppf)Cl₂·DCM (58 mg, 0.07102 mmol) and TEA (450 μL, 3.229 mmol) in MeOH (6 mL) in a steel bomb equipped with a stir bar was purged with carbon monoxide (2 g, 68.92 mmol) three times. The reaction mixture was heated to 100° C. with 150 psi of carbon monoxide and stirred for 14 h. The reaction mixture was allowed to cool to rt. The reaction mixture was filtered through a pad of celite, washed with ethyl acetate, filtrate was concentrated in vacuo. The residue was purified by silica gel chromatography (12 g of silica) using a gradient eluent of 100% hexanes to 50% ethyl acetate in hexanes to give as a white solid methyl 6-(1-methylcyclopropyl)furo[2,3-b]pyrazine-2-carboxylate (77 mg, 48%), ¹H NMR (400 MHz, Chloroform-d) δ 8.94 (s, 1H), 6.71 (s, 1H), 4.05 (s, 3H), 1.56 (s, 3H), 1.47-1.42 (m, 2H), 1.03-0.99 (m, 2H). ESI-MS m/z calc. 232.0848, found 233.3 (M+1)⁺; Retention time: 1.44 minutes; LC method A.

### Step 4: 6-(1-Methylcyclopropyl)furo[2,3-b]pyrazine-2-carbaldehyde

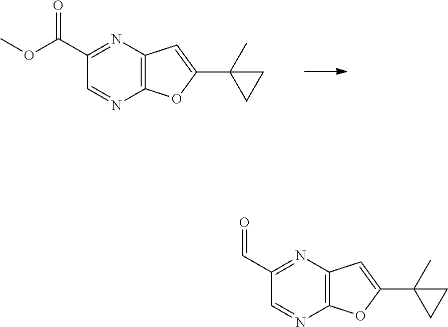 [SMILES: COC(=O)c1cnc2oc(C3(C)CC3)cc2n1 ; CC1(c2cc3nc(C=O)cnc3o2)CC1]

To a stirred solution of methyl 6-(1-methylcyclopropyl)furo[2,3-b]pyrazine-2-carboxylate (77 mg, 0.3316 mmol) in DCM (3 mL) was added DIBAL (in hexanes) (665 μL of 1 M, 0.6650 mmol) at −78° C. over 2 minutes. The reaction mixture was stirred for 4 h, quenched with MeOH (1 mL)/water (1 mL) and concentrated in vacuo. DCM (3 mL) was added and filtered. The cake was rinsed with DCM. The organic layer from the filtrate was separated, washed with brine (2 mL), dried over sodium sulfate, filtered and concentrated in vacuo. The residue was purified by silica gel chromatography (4 g of silica) by using 0-50% ethyl acetate in hexanes to afford 6-(1-methylcyclopropyl)furo[2,3-b]pyrazine-2-carbaldehyde (43 mg, 63%), ¹H NMR (400 MHz, Chloroform-d) δ 10.19 (s, 1H), 8.79 (s, 1H), 6.73 (s, 1H), 1.58 (s, 3H), 1.50-1.43 (m, 2H), 1.07-0.99 (m, 2H). ESI-MS m/z calc. 202.07423, found 203.2 (M+1)⁺; Retention time: 1.37 minutes; LC method A.

### Step 5: 3-[[4-(2,6-Dimethylphenyl)-6-[(2R)-3-(1-methylcyclopropyl)-2-[[6-(1-methylcyclopropyl)furo[2,3-b]pyrazin-2-yl]methylamino]propoxy]pyrimidin-2-yl]sulfamoyl]benzoic acid

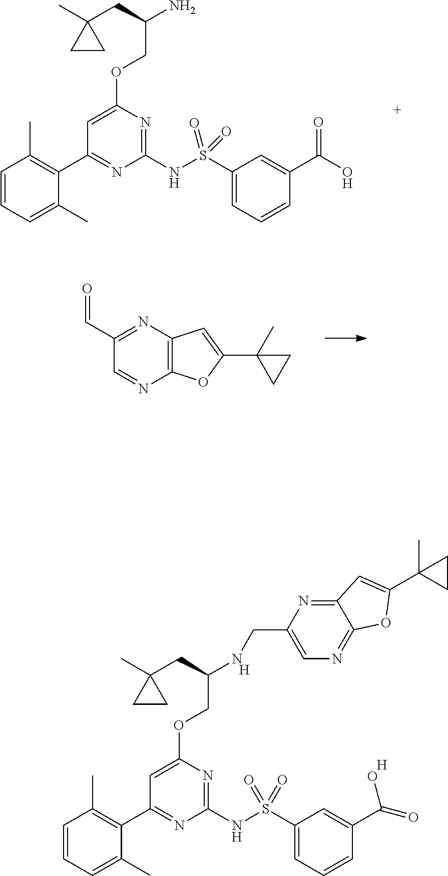

A 20 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (62 mg, 0.1130 mmol), 6-(1-methylcyclopropyl)furo[2,3-b]pyrazine-2-carbaldehyde (24.0 mg, 0.1175 mmol), anhydrous DCM (750 μL), acetic acid (15 μL, 0.2638 mmol) and DIEA (70 μL, 0.4019 mmol). The mixture was cooled down in an ice bath, sodium triacetoxyborohydride (83 mg, 0.3916 mmol) was added and the reaction was stirred for 1 h. The reaction was quenched with aqueous 1 M HCl (1 mL), MeOH (0.5 mL) and DMSO (0.5 mL), filtered and purified by reverse phase HPLC (1-99% acetonitrile/5 mM aqueous HCl over 15 min) to give as a white solid 3-[[4-(2,6-dimethylphenyl)-6-[(2R)-3-(1-methylcyclopropyl)-2-[[6-(1-methylcyclopropyl)furo[2,3-b]pyrazin-2-yl]methylamino]propoxy]pyrimidin-2-yl]sulfamoyl]benzoic acid (hydrochloride salt) (58.6 mg, 71%). ESI-MS m/z calc. 696.273, found 697.3 (M+1)⁺; Retention time: 1.5 minutes. LC method A.

### Step 6: (11R)-6-(2,6-Dimethylphenyl)-12-[[6-(1-methylcyclopropyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-96

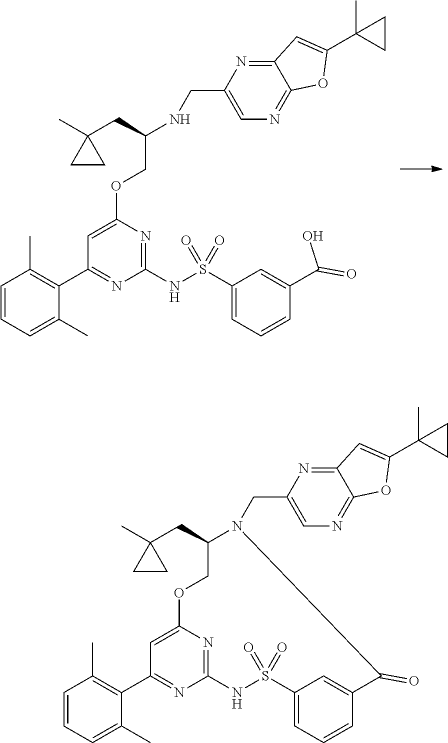 [SMILES: Cc1cccc(C)c1-c1cc(OC[C@@H](CC2(C)CC2)NCc2cnc3oc(C4(C)CC4)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(C4(C)CC4)cc3n1)[C@H](CC1(C)CC1)CO2]

3-[[4-(2,6-dimethylphenyl)-6-[(2R)-3-(1-methylcyclopropyl)-2-[[6-(1-methylcyclopropyl)furo[2,3-b]pyrazin-2-yl]methylamino]propoxy]pyrimidin-2-yl]sulfamoyl]benzoic acid (hydrochloride salt) (58.60 mg, 0.07992 mmol) was combined under nitrogen with CDMT (38 mg, 0.2164 mmol) and DMF (1.6 mL). The solution was stirred at 0° C. 4-Methyl-morpholine (55 μL, 0.5003 mmol) was added and the mixture was stirred in the cooling bath that was allowed to warm to room temperature. After 6 h, the reaction was filtered and purified by reverse phase HPLC (1-99% acetonitrile/5 mM aqueous HCl over 30 min) to give as a white solid (11R)-6-(2,6-dimethylphenyl)-12-[[6-(1-methylcyclopropyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (34.4 mg, 63%), ¹H NMR (400 MHz, Chloroform-d) δ 8.73 (t, J=1.8 Hz, 1H), 8.47 (s, 1H), 8.34 (s, 1H), 8.07 (dt, J=8.1, 1.4 Hz, 1H), 7.86 (dt, J=7.8, 1.3 Hz, 1H), 7.63 (t, J=7.8 Hz, 1H), 7.21 (t, J=7.6 Hz, 1H), 7.06 (d, J=7.6 Hz, 2H), 6.64 (s, 1H), 6.25 (s, 1H), 5.39 (dd, J=11.2, 4.2 Hz, 1H), 5.27 (d, J=14.8 Hz, 1H), 4.46-4.36 (m, 1H), 4.28 (t, J=11.5 Hz, 1H), 4.15 (d, J=14.8 Hz, 1H), 2.01 (s, 6H), 1.81 (d, J=15.3 Hz, 1H), 1.54 (s, 3H), 1.48 (dd, J=15.1, 9.9 Hz, 1H), 1.42-1.38 (m, 2H), 0.98-0.91 (m, 2H), 0.48 (s, 3H), 0.38-0.31 (m, 1H), 0.28-0.20 (m, 1H), 0.16-0.08 (m, 1H), 0.05-0.00 (m, 1H). ESI-MS m/z calc. 678.26245, found 679.2 (M+1)⁺; Retention time: 2.17 minutes. LC method A.

### Example 60: Preparation of Compound III-13 and Compound III-14

### Step 1: (11R)-6-(2,6-dimethylphenyl)-12-[(6-isopropyl-6,7-dihydrofuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one, isomer 1, Compound III-13, and (11R)-6-(2,6-dimethylphenyl)-12-[(6-isopropyl-6,7-dihydrofuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one, isomer 2, Compound III-14

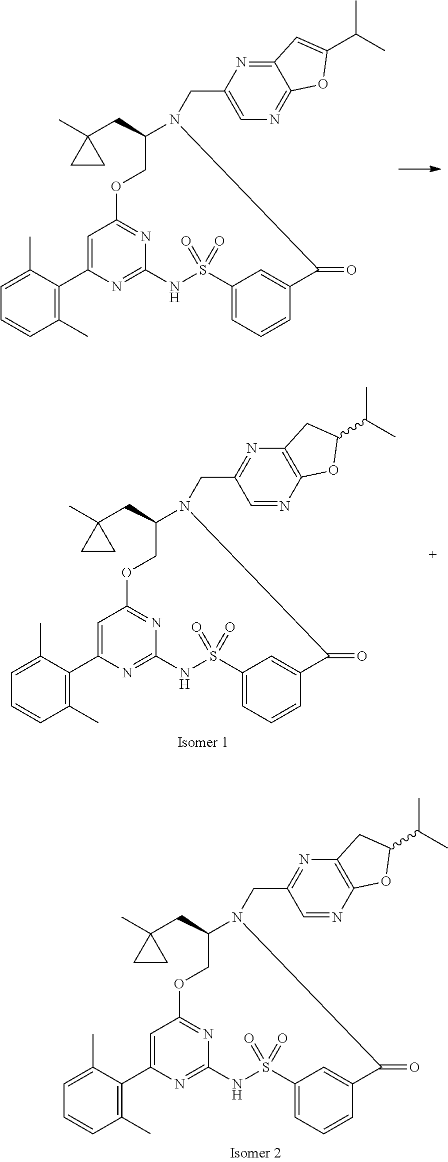 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2 ; Cc1cccc(C)c1-c1cc2nc(n1)NS(=O)(=O)c1cccc(c1)C(=O)N(Cc1cnc3c(n1)CC(C(C)C)O3)[C@H](CC1(C)CC1)CO2 ; Cc1cccc(C)c1-c1cc2nc(n1)NS(=O)(=O)c1cccc(c1)C(=O)N(Cc1cnc3c(n1)CC(C(C)C)O3)[C@H](CC1(C)CC1)CO2]

25%R)-6-(2,6-dimethylphenyl)-12-[(6-isopropylfuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (35 mg, 0.05144 mmol) in methanol (2 mL) was added palladium on carbon (13 mg of 10% w/w, 0.01222 mmol) (Degussa type E101 NE/W) under nitrogen. The capped vial was stirred at 60° C. overnight (20 h) under hydrogen atmosphere (balloon). The reaction was allowed to cool to ambient temperature and purged with nitrogen. The heterogeneous mixture was filtered over a pad of Celite and concentrated under reduced pressure. Purification of the crude by preparative reverse-phase HPLC (1-99% acetonitrile in water over 30 min, 5 mM HCl as modifier) furnished partially separated two diastereomers as white solids. Isomer 1 (more polar): white solid. (11R)-6-(2,6-dimethylphenyl)-12-[(6-isopropyl-6,7-dihydrofuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (5.5 mg, 16%) ¹H NMR (400 MHz, CDCl₃) δ 9.04 (s, 1H), 8.69 (t, J=1.8 Hz, 1H), 8.12 (t, J=1.1 Hz, 1H), 8.00 (dt, J=8.2, 1.3 Hz, 1H), 7.85 (dt, J=7.7, 1.4 Hz, 1H), 7.61 (t, J=7.8 Hz, 1H), 7.21 (t, J=7.6 Hz, 1H), 7.06 (d, J=7.6 Hz, 2H), 6.23 (s, 1H), 5.34 (dd, J=11.2, 4.2 Hz, 1H), 5.08 (d, J=14.8 Hz, 1H), 4.67 (ddd, J=9.1, 8.0, 7.1 Hz, 1H), 4.42-4.32 (m, 1H), 4.23 (t, J=11.5 Hz, 1H), 3.95 (d, J=14.8 Hz, 1H), 3.35 (ddd, J=17.6, 9.1, 1.0 Hz, 1H), 3.14 (ddd, J=17.8, 8.1, 1.2 Hz, 1H), 2.11-2.02 (m, 1H), 2.00 (s, 6H), 1.78 (d, J=15.1 Hz, 1H), 1.43 (dd, J=15.1, 10.0 Hz, 1H), 1.09 (d, J=6.6 Hz, 3H), 1.02 (d, J=6.8 Hz, 3H), 0.47 (s, 3H), 0.35-0.28 (m, 1H), 0.26-0.19 (m, 1H), 0.15-0.07 (m, 1H), −0.00-−0.05 (m, 1H). ESI-MS m/z calc. 668.2781, found 669.7 (M+1)⁺; Retention time: 1.92 minutes. Isomer 2 (less polar): white solid. (11R)-6-(2,6-dimethylphenyl)-12-[(6-isopropyl-6,7-dihydrofuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (2.9 mg, 8%) ¹H NMR (400 MHz, CDCl₃) δ 8.90-8.45 (br, 1H), 8.68 (t, J=1.8 Hz, 1H), 8.12 (t, J=1.1 Hz, 1H), 8.02 (dt, J=8.2, 1.3 Hz, 1H), 7.86 (dt, J=7.7, 1.4 Hz, 1H), 7.62 (t, J=7.8 Hz, 1H), 7.21 (t, J=7.6 Hz, 1H), 7.07 (d, J=7.6 Hz, 2H), 6.25 (s, 1H), 5.38 (dd, J=11.1, 4.0 Hz, 1H), 5.04 (d, J=14.7 Hz, 1H), 4.71 (ddd, J=9.1, 8.0, 6.7 Hz, 1H), 4.42-4.33 (m, 1H), 4.27 (t, J=11.4 Hz, 1H), 3.96 (d, J=14.8 Hz, 1H), 3.39 (ddd, J=17.7, 9.1, 1.0 Hz, 1H), 3.10 (ddd, J=17.8, 8.0, 1.2 Hz, 1H), 2.10-2.03 (m, 1H), 2.02 (s, 6H), 1.79 (d, J=15.2 Hz, 1H), 1.42 (dd, J=15.1, 9.9 Hz, 1H), 1.08 (d, J=6.7 Hz, 3H), 1.02 (d, J=6.8 Hz, 3H), 0.46 (s, 3H), 0.35-0.28 (m, 1H), 0.26-0.19 (m, 1H), 0.16-0.08 (m, 1H), −0.00-−0.05 (m, 1H). ESI-MS m/z calc. 668.2781, found 669.6 (M+1)⁺; Retention time: 1.95 minutes. LC method A.

### Example 61: Preparation of Compound I-98

### Step 1: 5-Chloro-3-(3,3-dimethylbut-1-ynyl)-1-[(4-methoxyphenyl)methyl]pyrazin-2-one

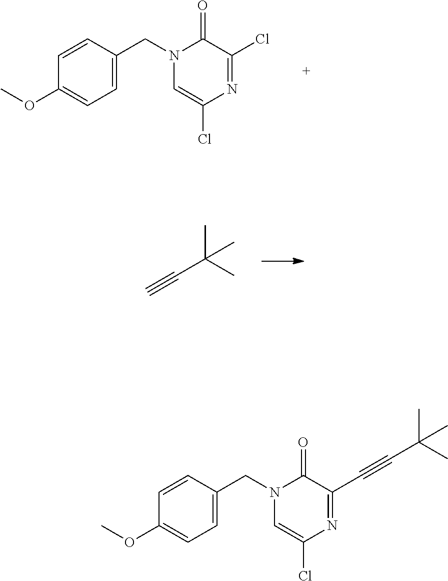

To a 20 mL microwave vial was charged 3,5-dichloro-1-[(4-methoxyphenyl)methyl]pyrazin-2-one (457 mg, 1.603 mmol), Bis(triphenylphosphine)palladium(II) dichloride (21 mg, 0.02992 mmol), CuI (14 mg, 0.07351 mmol) in DMF (3.5 mL) and TEA (1.3 mL, 9.327 mmol). Then, 3,3-dimethylbut-1-yne (290 μL, 2.429 mmol) was added and the vessel was sealed. The mixture was irradiated at 80° C. μW for 10 min. The reaction mixture was allowed to cool, diluted with DCM (20 mL) and washed with water (2×10 mL). The organic layer was washed with brine (10 mL), dried with sodium sulfate, filtered and concentrated under vacuo. The resulting material was purified by silica gel chromatography (24 g of silica) using a gradient eluent of 100% hexanes to 80% ethyl acetate in hexanes to afford as a yellow oil 5-chloro-3-(3,3-dimethylbut-1-ynyl)-1-[(4-methoxyphenyl)methyl]pyrazin-2-one (457 mg, 86%), ¹H NMR (400 MHz, Methanol-d4) δ 7.77 (s, 1H), 7.38-7.29 (m, 2H), 6.96-6.88 (m, 2H), 5.05 (s, 2H), 3.78 (s, 3H), 1.34 (s, 9H). ESI-MS m/z calc. 330.1135, found 331.2 (M+1)⁺; Retention time: 1.92 minutes; LC method A.

### Step 2: 6-tert-Butyl-2-chloro-furo[2,3-b]pyrazine

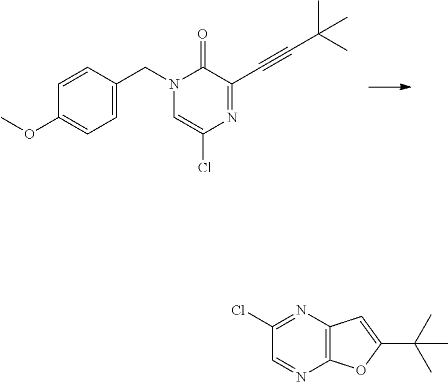 [SMILES: COc1ccc(Cn2cc(Cl)nc(C#CC(C)(C)C)c2=O)cc1 ; CC(C)(C)c1cc2nc(Cl)cnc2o1]

To a solution of 5-chloro-3-(3,3-dimethylbut-1-ynyl)-1-[(4-methoxyphenyl)methyl]pyrazin-2-one (453 mg, 1.369 mmol) in DCM (9.0 mL), Trifluoromethanesulfonate (18 mg, 0.07006 mmol) (silver salt) and TFA (950 μL, 12.33 mmol) were added and stirred at room temperature for 90 minutes. The residue was concentrated in vacuo and purified by silica gel chromatography (40 g of silica) using a gradient eluent of 100% hexanes to 30% ethyl acetate in hexanes to give as an off-white solid 6-tert-butyl-2-chloro-furo[2,3-b]pyrazine (258.0 mg, 89%), ¹H NMR (400 MHz, Methanol-d4) δ 8.21 (s, 1H), 6.73 (s, 1H), 1.43 (s, 9H). ESI-MS m/z calc. 210.05598, found 211.2 (M+1)⁺; Retention time: 1.9 minutes. LC method A.

### Step 3: Methyl 6-tert-butylfuro[2,3-b]pyrazine-2-carboxylate

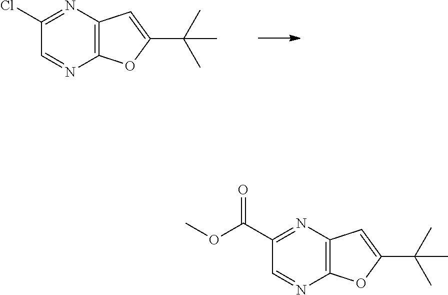 [SMILES: CC(C)(C)c1cc2nc(Cl)cnc2o1 ; COC(=O)c1cnc2oc(C(C)(C)C)cc2n1]

A mixture of 6-tert-butyl-2-chloro-furo[2,3-b]pyrazine (295 mg, 1.400 mmol) and Pd(dppf)Cl₂·DCM (115 mg, 0.1408 mmol) and TEA (900 μL, 6.457 mmol) in MeOH (10 mL) in a steel pressure vessel equipped with a stir bar was purged with carbon monoxide (4 g, 137.8 mmol) three times. The reaction mixture was heated to 100° C. with 120 psi of carbon monoxide and stirred for 14 h. The reaction mixture was allowed to cool to rt. The reaction mixture was filtered through a pad of celite, washed with ethyl acetate, filtrate was concentrated in vacuo. The residue was purified by silica gel chromatography (40 g of silica) using a gradient eluent of 100% hexanes to 50% ethyl acetate in hexanes to give as a white solid methyl 6-tert-butylfuro[2,3-b]pyrazine-2-carboxylate (238 mg, 73%) ¹H NMR (400 MHz, Chloroform-d) δ 9.01 (s, 1H), 6.69 (s, 1H), 4.06 (s, 3H), 1.45 (s, 9H). ESI-MS m/z calc. 234.10045, found 235.3 (M+1)⁺; Retention time: 1.53 minutes. LC method A.

### Step 4: 6-tert-Butylfuro[2,3-b]pyrazine-2-carbaldehyde

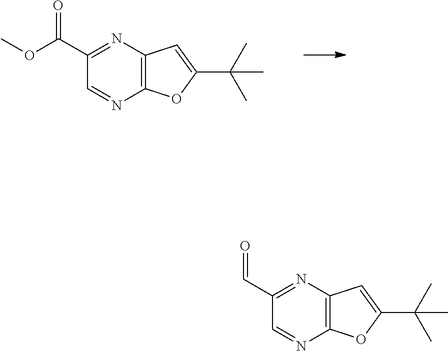 [SMILES: COC(=O)c1cnc2oc(C(C)(C)C)cc2n1 ; CC(C)(C)c1cc2nc(C=O)cnc2o1]

To a stirred solution of methyl 6-tert-butylfuro[2,3-b]pyrazine-2-carboxylate (235 mg, 1.003 mmol) in DCM (8 mL) was added DIBAL (in hexanes) (2.00 mL of 1 M, 2.000 mmol) at −78° C. over 2 minutes. The reaction mixture was stirred for 2.5 h, quenched with MeOH (3 mL)/water (3 mL) and concentrated in vacuo. DCM (10 mL) was added and filtered. The cake was rinsed with DCM. The organic layer from the filtrate was separated, washed with brine (2 mL), dried over sodium sulfate, filtered and concentrated in vacuo. The residue was purified by silica gel chromatography (24 g of silica) by using 0-50% ethyl acetate in hexanes to afford 6-tert-butylfuro[2,3-b]pyrazine-2-carbaldehyde (156.2 mg, 76%), ¹H NMR (400 MHz, Chloroform-d) δ 10.20 (s, 1H), 8.86 (s, 1H), 6.72 (s, 1H), 1.46 (s, 9H). ESI-MS m/z calc. 204.08987, found 205.2 (M+1)⁺; Retention time: 1.55 minutes. LC method A.

### Step 5: 3-[[4-[(2R)-2-[(6-tert-Butylfuro[2,3-b]pyrazin-2-yl)methylamino]-3-(1-methylcyclopropyl)propoxy]-6-(2,6-dimethylphenyl)pyrimidin-2-yl]sulfamoyl]benzoic acid

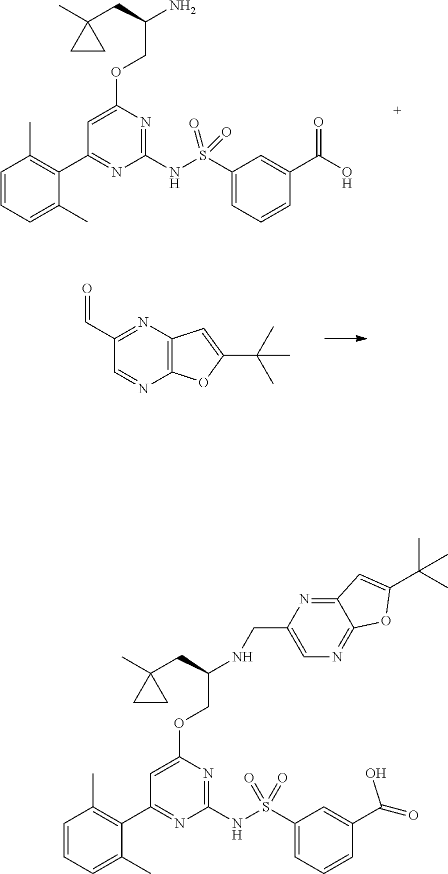 [SMILES: Cc1cccc(C)c1-c1cc(OC[C@H](N)CC2(C)CC2)nc(NS(=O)(=O)c2cccc(C(=O)O)c2)n1 ; CC(C)(C)c1cc2nc(C=O)cnc2o1 ; Cc1cccc(C)c1-c1cc(OC[C@@H](CC2(C)CC2)NCc2cnc3oc(C(C)(C)C)cc3n2)nc(NS(=O)(=O)c2cccc(C(=O)O)c2)n1]

A 20 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (80 mg, 0.1458 mmol), 6-tert-butylfuro[2,3-b]pyrazine-2-carbaldehyde (31.3 mg, 0.1533 mmol), anhydrous DCM (3.0 mL), acetic acid (20 μL, 0.3517 mmol) and DIEA (95 μL, 0.5454 mmol). The mixture was cooled down in an ice bath, sodium triacetoxyborohydride (108 mg, 0.5096 mmol) was added and the reaction was stirred for 3 h allowing to warm to room temperature. The reaction was quenched with aqueous 1 M HCl (1 mL), MeOH (0.5 mL) and DMSO (0.5 mL), filtered and purified by reverse phase HPLC (1-99% acetonitrile/5 mM aqueous HCl over 30 min) to give as a white solid 3-[[4-[(2R)-2-[(6-tert-butylfuro[2,3-b]pyrazin-2-yl)methylamino]-3-(1-methyl cyclopropyl)propoxy]-6-(2,6-dimethylphenyl)pyrimidin-2-yl]sulfamoyl]benzoic acid (hydrochloride salt) (65.4 mg, 61%). ESI-MS m/z calc. 698.28864, found 699.2 (M+1)⁺; Retention time. 1.54 minutes. LC method A.

### Step 6: (11R)-12-[(6-tert-Butylfuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one(Compound I-98

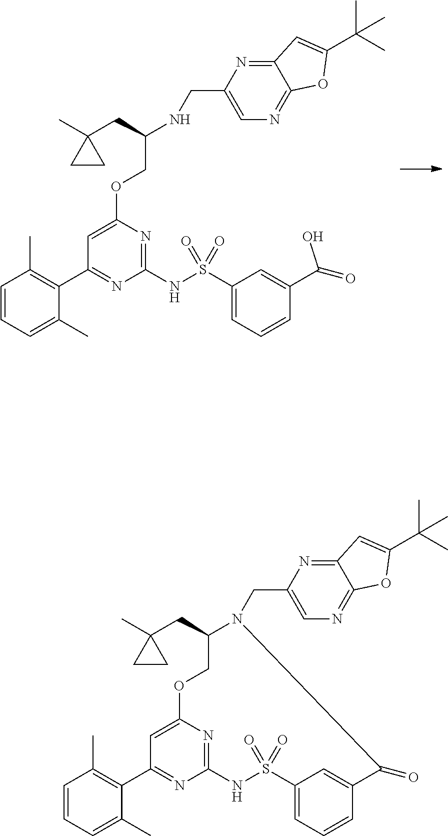 [SMILES: Cc1cccc(C)c1-c1cc(OC[C@@H](CC2(C)CC2)NCc2cnc3oc(C(C)(C)C)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(C(C)(C)C)cc3n1)[C@H](CC1(C)CC1)CO2]

3-[[4-[(2R)-2-[(6-tert-butylfuro[2,3-b]pyrazin-2-yl)methylamino]-3-(1-methylcyclopropyl)propoxy]-6-(2,6-dimethylphenyl)pyrimidin-2-yl]sulfamoyl]benzoic acid (hydrochloride salt) (65.4 mg, 0.08894 mmol) was combined under nitrogen with CDMT (43 mg, 0.2449 mmol) and DMF (2.0 mL). The solution was stirred at 0° C. 4-Methyl-morpholine (65 μL, 0.5912 mmol) was added and the mixture was stirred in the cooling bath that was allowed to warm to room temperature. After 16 h, the reaction was filtered and purified by reverse phase HPLC (1-99% acetonitrile/5 mM aqueous HCl over 30 min) to give as a white solid (11R)-12-[(6-tert-butylfuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (33.4 mg, 55%), ¹H NMR (400 MHz, Chloroform-d) δ 8.73 (t, J=1.8 Hz, 1H), 8.41 (s, 1H), 8.33 (s, 1H), 8.07 (dt, J=8.2, 1.3 Hz, 1H), 7.87 (dt, J=7.7, 1.4 Hz, 1H), 7.64 (t, J=7.8 Hz, 1H), 7.24-7.17 (m, 1H), 7.07 (d, J=7.6 Hz, 2H), 6.62 (s, 1H), 6.26 (s, 1H), 5.40 (dd, J=11.2, 4.2 Hz, 1H), 5.31-5.24 (m, 1H), 4.47-4.38 (m, 1H), 4.31 (t, J=11.5 Hz, 1H), 4.16 (d, J=14.8 Hz, 1H), 2.02 (s, 6H), 1.82 (d, J=15.0 Hz, 1H), 1.51-1.45 (m, 1H), 1.43 (s, 9H), 0.48 (s, 3H), 0.41-0.33 (m, 1H), 0.28-0.20 (m, 1H), 0.16-0.08 (m, 1H), 0.04-0.01 (m, 1H). ESI-MS m/z calc. 680.2781, found 681.2 (M+1)⁺; Retention time: 2.21 minutes. LC method A.

### Example 62: Preparation of Compound I-99

### Step 1: 3-[[4-[(2R)-3-(1-Bicyclo[1.1.1]pentanyl)-2-[(6-tert-butylfuro[2,3-b]pyrazin-2-yl)methylamino]propoxy]-6-(2,6-dimethylphenyl)pyrimidin-2-yl]sulfamoyl]benzoic acid

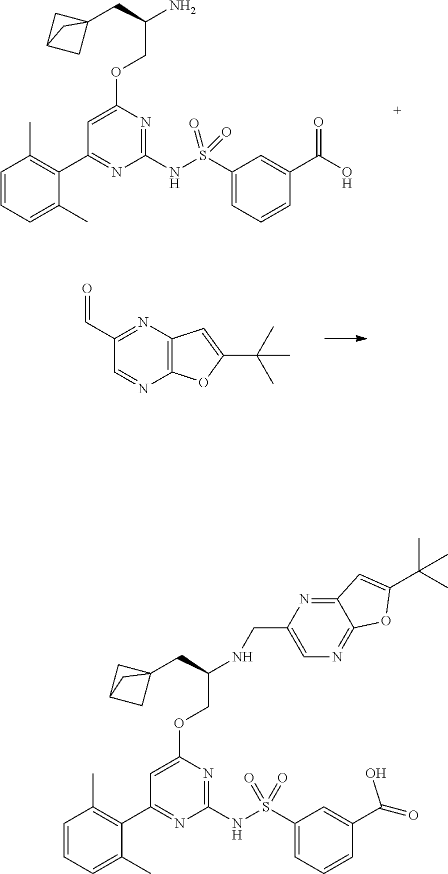 [SMILES: Cc1cccc(C)c1-c1cc(OC[C@H](N)CC23CC(C2)C3)nc(NS(=O)(=O)c2cccc(C(=O)O)c2)n1 ; CC(C)(C)c1cc2nc(C=O)cnc2o1 ; Cc1cccc(C)c1-c1cc(OC[C@@H](CC23CC(C2)C3)NCc2cnc3oc(C(C)(C)C)cc3n2)nc(NS(=O)(=O)c2cccc(C(=O)O)c2)n1]

A 20 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (83 mg, 0.1476 mmol), 6-tert-butylfuro[2,3-b]pyrazine-2-carbaldehyde (31.6 mg, 0.1547 mmol), anhydrous DCM (3.0 mL), acetic acid (20 μL, 0.3517 mmol) and DIEA (95 μL, 0.5454 mmol). The mixture was cooled down in an ice bath, sodium triacetoxyborohydride (110 mg, 0.5190 mmol) was added and the reaction was stirred for 4 h allowing to warm to room temperature. The reaction was quenched with aqueous 1 M HCl (1 mL), MeOH (0.5 mL) and DMSO (0.5 mL), filtered and purified by reverse phase HPLC (1-99% acetonitrile/5 mM aqueous HCl over 30 min) to give as a white solid 3-[[4-[(2R)-3-(1-bicyclo[1.1.1]pentanyl)-2-[(6-tert-butylfuro[2,3-b]pyrazin-2-yl)methylamino]propoxy]-6-(2,6-dimethylphenyl)pyrimidin-2-yl]sulfamoyl]benzoic acid (hydrochloride salt) (68.3 mg, 62%). ESI-MS m/z calc. 710.28864, found 711.2 (M+1)⁺; Retention time: 1.61 minutes. LC method A.

### Step 2: (11R)-11-(1-Bicyclo[1.1.1]pentanylmethyl)-12-[(6-tert-butylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-99

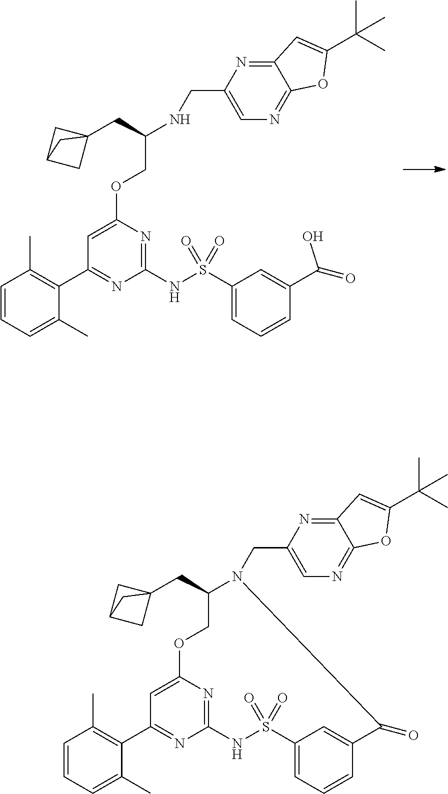 [SMILES: Cc1cccc(C)c1-c1cc(OC[C@@H](CC23CC(C2)C3)NCc2cnc3oc(C(C)(C)C)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(C(C)(C)C)cc3n1)[C@H](CC13CC(C1)C3)CO2]

3-[[4-[(2R)-3-(1-bicyclo[1.1.1]pentanyl)-2-[(6-tert-butylfuro[2,3-b]pyrazin-2-yl)methylamino]propoxy]-6-(2,6-dimethylphenyl)pyrimidin-2-yl]sulfamoyl]benzoic acid (hydrochloride salt) (67 mg, 0.08966 mmol) was combined under nitrogen with CDMT (43 mg, 0.2449 mmol) and DMF (3.0 mL). The solution was stirred at 0° C. 4-Methyl-morpholine (65 μL, 0.5912 mmol) was added and the mixture was stirred in the cooling bath that was allowed to warm to room temperature. After 16 h, the reaction was filtered and purified by reverse phase HPLC (1-99% acetonitrile/5 mM aqueous HCl over 30 min) to give as a white solid (11R)-11-(1-bicyclo[1.1.1]pentanylmethyl)-12-[(6-tert-butylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (33.6 mg, 54%), ¹H NMR (400 MHz, Chloroform-d) δ 9.06 (s, 1H), 8.72 (t, J=1.8 Hz, 1H), 8.41 (s, 1H), 8.14 (d, J=7.9 Hz, 1H), 7.89 (dt, J=7.7, 1.4 Hz, 1H), 7.67 (t, J=7.8 Hz, 1H), 7.20 (t, J=7.6 Hz, 1H), 7.04 (d, J=7.6 Hz, 2H), 6.61 (s, 1H), 6.20 (s, 1H), 5.40 (dd, J=11.4, 4.0 Hz, 1H), 5.36-5.29 (m, 1H), 4.29-4.18 (m, 2H), 4.09-4.00 (m, 1H), 2.45 (s, 1H), 2.09-1.96 (m, 7H), 1.81 (dd, J=15.5, 3.3 Hz, 1H), 1.62-1.56 (m, 6H), 1.42 (s, 9H). ESI-MS m/z calc. 692.2781, found 693.2 (M+1)⁺; Retention time: 2.3 minutes. LC method A.

### Example 63: Preparation of Compound I-104

### Step 1: (2R)-2-Amino-3-cyclobutyl-propan-1-ol

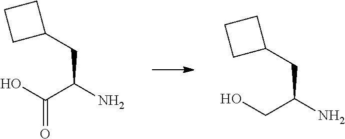 [SMILES: N[C@H](CC1CCC1)C(=O)O ; N[C@@H](CO)CC1CCC1]

Borane THF complex (367 mL of 1 M, 367.00 mmol) was slowly added to a suspension of (2R)-2-amino-3-cyclobutyl-propanoic acid (25 g, 174.60 mmol) in 2-methyltetrahydrofuran (250 mL) at room temperature. The reaction mixture was stirred at room temperature for 20 h. Aqueous HCl (176 mL of 3 M, 528.00 mmol) was then added while keeping the temperature below 25° C. The solution was stirred for 45 minutes. Excess THF (around 350 mL) was evaporated. 2-Methyltetrahydrofuran (150 mL) was then added. The solution was basified at pH around 9 with a 25% aqueous NaOH solution (around 125 mL). The organic phase was separated. The aqueous phase was extracted with MeTHF (2×125 mL). The combined organic phases were washed with brine (125 mL) then dried over sodium sulfate, filtered, and concentrated. The residue was dried over high vacuum to give (2R)-2-amino-3-cyclobutyl-propan-1-ol (25.03 g, 111%) as a colorless oil. ESI-MS m/z calc. 129.11537, found 130.2 (M+1)⁺; Retention time: 1.71 minutes; LC method K.

### Step 2: 3-[[4-[(2R)-2-Amino-3-cyclobutyl-propoxy]-6-(2,6-dimethylphenyl)pyrimidin-2-yl]sulfamoyl]benzoic acid

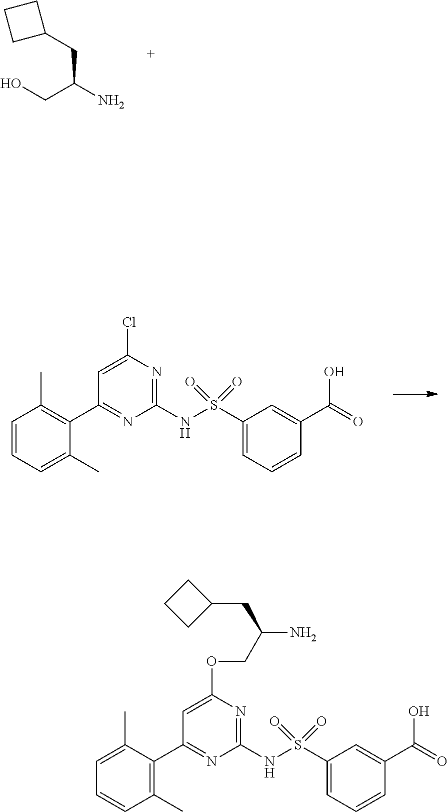 [SMILES: N[C@@H](CO)CC1CCC1 ; Cc1cccc(C)c1-c1cc(Cl)nc(NS(=O)(=O)c2cccc(C(=O)O)c2)n1 ; Cc1cccc(C)c1-c1cc(OC[C@H](N)CC2CCC2)nc(NS(=O)(=O)c2cccc(C(=O)O)c2)n1]

A solution of (2R)-2-amino-3-cyclobutyl-propan-1-ol (4.0 g, 29.412 mmol) in anhydrous DMF (12 mL) was added to a solution of 3-[[4-chloro-6-(2,6-dimethylphenyl)pyrimidin-2-yl]sulfamoyl]benzoic acid (12.9 g, 30.871 mmol) in 2-methyltetrahydrofuran (110 mL). The mixture was then cooled down to 10-15° C. and sodium tert-butoxide (17 g, 176.89 mmol) was added. The reaction was stirred at 10-15° C. for 2 h, then cooled down to 0° C. and quenched by the addition of an aqueous solution of iN hydrochloric acid (300 mL). The biphasic mixture was stirred for 15 minutes. The layers were then separated, and the aqueous layer was extracted with 2-methyltetrahydrofuran (5×250 mL). The combined organic layers were washed with brine (400 mL), dried over magnesium sulfate, filtered, and concentrated under reduced pressure to afford a beige foam which was triturated overnight with ethyl acetate (300 mL). The solid was filtered, washed with ethyl acetate (3×50 mL), methyl-tert-butylether (3×50 mL), dried under high vacuum to afford 3-[[4-[(2R)-2-amino-3-cyclobutyl-propoxy]-6-(2,6-dimethylphenyl)pyrimidin-2-yl]sulfamoyl]benzoic acid (hydrochloride salt) (12.11 g, 71%) as a white powder which could be used without further purification. ¹H NMR (400 MHz, DMSO-d6) δ 13.32 (br. s, 1H), 8.46 (t, J=1.5 Hz, 1H), 8.29-7.90 (m, 4H), 7.71 (t, J=7.8 Hz, 1H), 7.32-7.21 (m, 1H), 7.19-7.06 (m, 2H), 6.30 (br. s, 1H), 4.31 (dd, J=12.2, 2.2 Hz, 1H), 4.10 (dd, J=11.9, 6.0 Hz, 1H), 3.46-3.27 (m, 1H, overlapped with water), 2.36 (quin, J=7.3 Hz, 1H, overlapped with DMSO), 2.19-1.50 (m, 14H). ESI-MS m/z calc. 510.1937, found 511.2 (M+1)⁺; Retention time: 2.36 minutes; LC method J.

### Step 3: 3-[[4-[(2R)-3-Cyclobutyl-2-[(6-cyclopropylfuro[2,3-b]pyrazin-2-yl)methylamino]propoxy]-6-(2,6-dimethylphenyl)pyrimidin-2-yl]sulfamoyl]benzoic acid

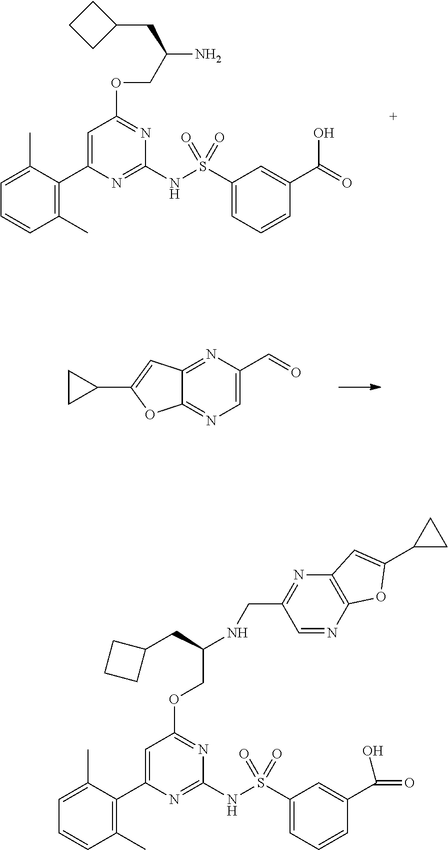 [SMILES: Cc1cccc(C)c1-c1cc(OC[C@H](N)CC2CCC2)nc(NS(=O)(=O)c2cccc(C(=O)O)c2)n1 ; O=Cc1cnc2oc(C3CC3)cc2n1 ; Cc1cccc(C)c1-c1cc(OC[C@@H](CC2CCC2)NCc2cnc3oc(C4CC4)cc3n2)nc(NS(=O)(=O)c2cccc(C(=O)O)c2)n1]

In a 4 mL vial, to a stirred mixture of 3-[[4-[(2R)-2-amino-3-cyclobutyl-propoxy]-6-(2,6-dimethylphenyl)pyrimidin-2-yl]sulfamoyl]benzoic acid (hydrochloride salt) (45 mg, 0.08226 mmol) and 6-cyclopropylfuro[2,3-b]pyrazine-2-carbaldehyde (15.5 mg, 0.08237 mmol) in anhydrous dichloromethane (300 μL) were added glacial acetic acid (10 μL, 0.1758 mmol) and DIPEA (40 μL, 0.2296 mmol), in that order, at 0° C. under nitrogen. After 2 min, sodium triacetoxyborohydride (60 mg, 0.2831 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish product as a white solid. 3-[[4-[(2R)-3-cyclobutyl-2-[(6-cyclopropylfuro[2,3-b]pyrazin-2-yl)methylamino]propoxy]-6-(2,6-dimethylphenyl)pyrimidin-2-yl]sulfamoyl]benzoic acid (hydrochloride salt) (41 mg, 69%)/ESI-MS m/z calc. 682.2573, found 683.8 (M+1)⁺; Retention time: 1.4 minutes; LC method A.

### Step 4: (11R)-11-(Cyclobutylmethyl)-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-104

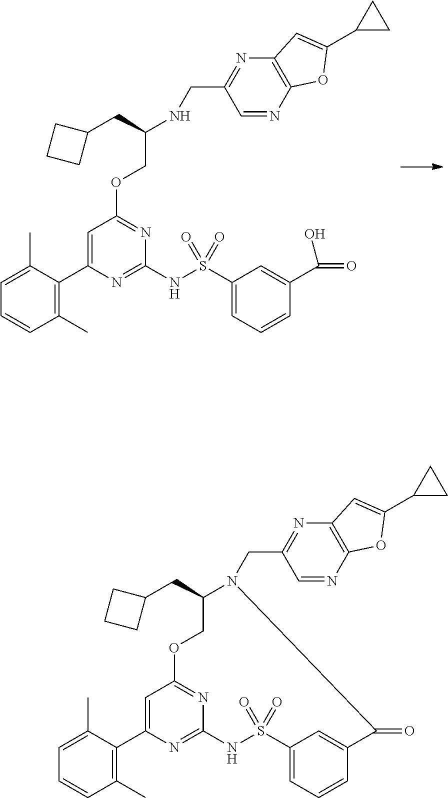 [SMILES: Cc1cccc(C)c1-c1cc(OC[C@@H](CC2CCC2)NCc2cnc3oc(C4CC4)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(C4CC4)cc3n1)[C@H](CC1CCC1)CO2]

To a stirred solution of 3-[[4-[(2R)-3-cyclobutyl-2-[(6-cyclopropylfuro[2,3-b]pyrazin-2-yl)methylamino]propoxy]-6-(2,6-dimethylphenyl)pyrimidin-2-yl]sulfamoyl]benzoic acid (hydrochloride salt) (41 mg, 0.05700 mmol) in anhydrous DMF (1.9 mL) was added 2-chloro-4,6-dimethoxy-1,3,5-triazine (16 mg, 0.09113 mmol) (CDMT), followed by addition of 4-methylmorpholine (40 μL, 0.3638 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished product as a white solid, (11R)-11-(cyclobutylmethyl)-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (28.2 mg, 74%) ¹H NMR (400 MHz, CDCl₃) δ 8.62 (s, 1H), 8.34 (s, 1H), 8.06 (d, J=7.9 Hz, 1H), 7.81 (dt, J=7.6, 1.4 Hz, 1H), 7.62 (t, J=7.8 Hz, 1H), 7.19 (t, J=7.7 Hz, 1H), 7.02 (d, J=7.6 Hz, 2H), 6.62 (s, 1H), 6.20 (s, 1H), 5.40-5.35 (m, 1H), 5.33 (d, J=15.0 Hz, 1H), 4.30-4.18 (m, 2H), 4.00 (tt, J=11.1, 3.9 Hz, 1H), 2.19-2.09 (m, 2H), 2.05-2.01 (m, 1H), 2.00 (s, 6H), 1.94-1.85 (m, 1H), 1.85-1.74 (m, 1H), 1.73-1.63 (m, 3H), 1.64-1.53 (m, 1H), 1.20-1.08 (m, 5H). ESI-MS m/z calc. 664.24677, found 665.8 (M+1)⁺; Retention time: 2.0 minutes. LC method A.

### Example 64: Preparation of Compound I-113

### Step 1: 3-(4-Benzyloxy-3,3-dimethyl-but-1-ynyl)-5-chloro-1-[(4-methoxyphenyl)methyl]pyrazin-2-one

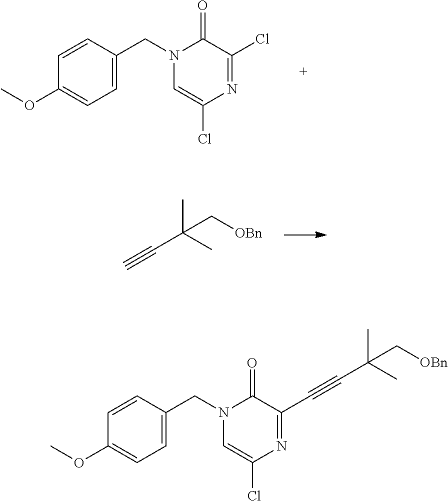 [SMILES: COc1ccc(Cn2cc(Cl)nc(Cl)c2=O)cc1 ; C#CC(C)(C)COCc1ccccc1 ; COc1ccc(Cn2cc(Cl)nc(C#CC(C)(C)COCc3ccccc3)c2=O)cc1]

To a 20 mL microwave vial was charged 3,5-dichloro-1-[(4-methoxyphenyl)methyl]pyrazin-2-one (750 mg, 2.630 mmol), Bis(triphenylphosphine)palladium(II) dichloride (35 mg, 0.04986 mmol), CuI (22 mg, 0.1155 mmol) in DMF (4.5 mL) and TEA (2.2 mL, 15.78 mmol). Then, 2,2-dimethylbut-3-ynoxymethylbenzene (745 mg, 3.957 mmol) was added and the vessel was sealed. The mixture was irradiated at 80° C. μW for 10 min. The reaction mixture was allowed to cool, diluted with DCM (20 mL) and washed with water (2×10 mL). The organic layer was washed with brine (10 mL), dried with sodium sulfate, filtered and concentrated under vacuo. The resulting material was purified by silica gel chromatography (24 g of silica) using a gradient eluent of 100% hexanes to 100% ethyl acetate to afford as a yellow oil 3-(4-benzyloxy-3,3-dimethyl-but-1-ynyl)-5-chloro-1-[(4-methoxyphenyl)methyl]pyrazin-2-one (930 mg, 81%) ESI-MS m/z calc. 436.15536, found 437.2 (M+1)⁺; Retention time: 2.13 minutes. LC method A.

### Step 2: 6-(2-Benzyloxy-1,1-dimethyl-ethyl)-2-chloro-furo[2,3-b]pyrazine

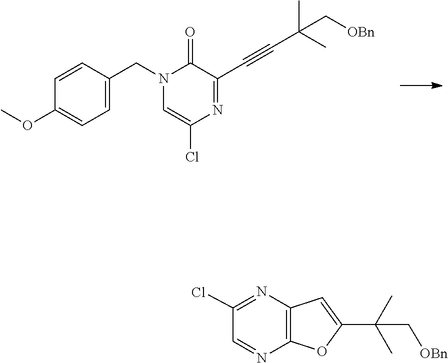 [SMILES: COc1ccc(Cn2cc(Cl)nc(C#CC(C)(C)COCc3ccccc3)c2=O)cc1 ; CC(C)(COCc1ccccc1)c1cc2nc(Cl)cnc2o1]

To a solution of 3-(4-benzyloxy-3,3-dimethyl-but-1-ynyl)-5-chloro-1-[(4-methoxyphenyl)methyl]pyrazin-2-one (930 mg, 2.128 mmol) in DCM (15 mL), Trifluoromethanesulfonate (15 mg, 0.05838 mmol) (silver salt) and TFA (1.5 mL, 19.47 mmol) were added and stirred at room temperature for 90 minutes. The residue was concentrated in vacuo and purified by silica gel chromatography (40 g of silica) using a gradient eluent of 100% hexanes to 30% ethyl acetate in hexanes to give an off-white oil which solidified under high vac to a white solid as 6-(2-benzyloxy-1,1-dimethyl-ethyl)-2-chloro-furo[2,3-b]pyrazine (603 mg, 89%), ¹H NMR (400 MHz, Methanol-d4) δ 8.20 (s, 1H), 7.28-7.16 (m, 5H), 6.76 (s, 1H), 4.48 (s, 2H), 3.64 (s, 2H), 1.42 (s, 6H). ESI-MS m/z calc. 316.09787, found 317.2 (M+1)⁺; Retention time: 2.17 minutes. LC method A.

### Step 3: Methyl 6-(2-benzyloxy-1,1-dimethyl-ethyl)furo[2,3-b]pyrazine-2-carboxylate

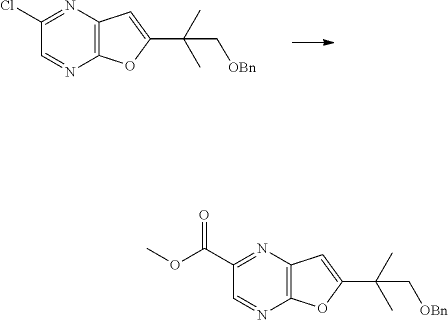 [SMILES: CC(C)(COCc1ccccc1)c1cc2nc(Cl)cnc2o1 ; COC(=O)c1cnc2oc(C(C)(C)COCc3ccccc3)cc2n1]

A mixture of 6-(2-benzyloxy-1,1-dimethyl-ethyl)-2-chloro-furo[2,3-b]pyrazine (603 mg, 1.904 mmol) and Pd(dppf)Cl₂·DCM (160 mg, 0.1959 mmol) and TEA (1.25 mL, 8.968 mmol) in MeOH (12 mL) in a steel pressure vessel equipped with a stir bar was purged with carbon monoxide (5.5 g, 189.5 mmol) three times. The reaction mixture was heated to 100° C. with 120 psi of carbon monoxide and stirred for 14 h. The reaction mixture was allowed to cool to rt. The reaction mixture was filtered through a pad of celite, washed with ethyl acetate, filtrate was concentrated in vacuo. The residue was purified by silica gel chromatography (80 g of silica) using a gradient eluent of 100% hexanes to 50% ethyl acetate in hexanes to give as a pale yellow solid methyl 6-(2-benzyloxy-1,1-dimethyl-ethyl)furo[2,3-b]pyrazine-2-carboxylate (539.0 mg, 83%) ¹H NMR (400 MHz, Chloroform-d) δ 9.01 (s, 1H), 7.30-7.19 (m, 5H), 6.80 (s, 1H), 4.50 (s, 2H), 4.06 (s, 3H), 3.63 (s, 2H), 1.45 (s, 6H). ESI-MS m/z calc. 340.1423, found 341.2 (M+1)⁺; Retention time: 1.9 minutes. LC method A.

### Step 4: 6-(2-Benzyloxy-1,1-dimethyl-ethyl)furo[2,3-b]pyrazine-2-carbaldehyde

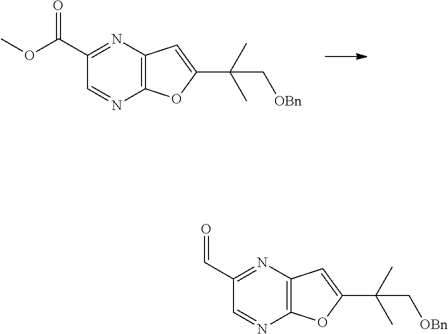 [SMILES: COC(=O)c1cnc2oc(C(C)(C)COCc3ccccc3)cc2n1 ; CC(C)(COCc1ccccc1)c1cc2nc(C=O)cnc2o1]

To a stirred solution of methyl 6-(2-benzyloxy-1,1-dimethyl-ethyl)furo[2,3-b]pyrazine-2-carboxylate (533 mg, 1.566 mmol) in DCM (20 mL) was added DIBAL (in hexanes) (3.1 mL of 1 M, 3.100 mmol) at −78° C. over 2 minutes. The reaction mixture was stirred for 4 h, then added an additional DIBAL (in hexanes) (1.2 mL of 1 M, 1.200 mmol) and allowed to stir 30 more minutes. The mixture was quenched with MeOH (6 mL)/water (6 mL) and concentrated in vacuo. DCM (30 mL) was added and filtered. The cake was rinsed with DCM. The organic layer from the filtrate was separated, washed with brine (5 mL), dried over sodium sulfate, filtered and concentrated in vacuo. The residue was purified by silica gel chromatography (40 g of silica) by using 0-50% ethyl acetate in hexanes to afford as an oil 6-(2-benzyloxy-1,1-dimethyl-ethyl)furo[2,3-b]pyrazine-2-carbaldehyde (329 mg, 68%), ¹H NMR (400 MHz, Chloroform-d) δ 10.20 (s, 1H), 8.86 (s, 1H), 7.30-7.21 (m, 5H) overlapped with proton from CHCl₃, 6.82 (s, 1H), 4.51 (s, 2H), 3.64 (s, 2H), 1.46 (s, 6H). ESI-MS m/z calc. 310.13174, found 311.2 (M+1)⁺; Retention time: 1.89 minutes. LC method A.

### Step 5: 3-[[4-[(2R)-2-[[6-(2-Benzyloxy-1,1-dimethyl-ethyl)furo[2,3-b]pyrazin-2-yl]methylamino]-3-(1-bicyclo[1.1.1]pentanyl)propoxy]-6-(2,6-dimethylphenyl)pyrimidin-2-yl]sulfamoyl]benzoic acid

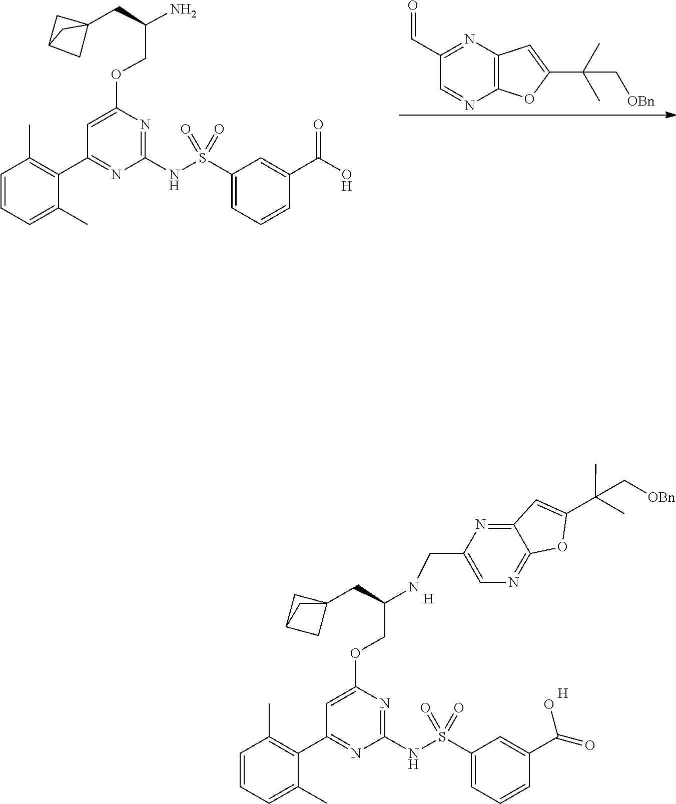

A 20 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (65 mg, 0.1156 mmol), 6-(2-benzyloxy-1,1-dimethyl-ethyl)furo[2,3-b]pyrazine-2-carbaldehyde (38 mg, 0.1224 mmol), anhydrous DCM (3.5 mL), acetic acid (20 μL, 0.3517 mmol) and DIEA (80 μL, 0.4593 mmol). The mixture was cooled down in an ice bath, sodium triacetoxyborohydride (89 mg, 0.4199 mmol) was added and the reaction was stirred for 3 h allowing to warm to room temperature. The reaction was quenched with aqueous 1 M HCl (1 mL), MeOH (0.5 mL) and DMSO (0.5 mL), filtered and purified by reverse phase HPLC (1-99% acetonitrile/5 mM aqueous HCl over 30 min) to give as a white solid 3-[[4-[(2R)-2-[[6-(2-benzyloxy-1,1-dimethyl-ethyl)furo[2,3-b]pyrazin-2-yl]methylamino]-3-(1-bicyclo[1.1.1]pentanyl)propoxy]-6-(2,6-dimethylphenyl)pyrimidin-2-yl]sulfamoyl]benzoic acid (hydrochloride salt) (66.8 mg, 68%). ESI-MS m/z calc. 816.3305, found 817.2 (M+1)⁺; Retention time: 1.8 minutes. LC method A.

### Step 6: (11R)-12-[[6-(2-Benzyloxy-1,1-dimethyl-ethyl)furo[2,3-b]pyrazin-2-yl]methyl]-11-(1-bicyclo[1.1.]pentanylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,4,16-hexaen-13-one

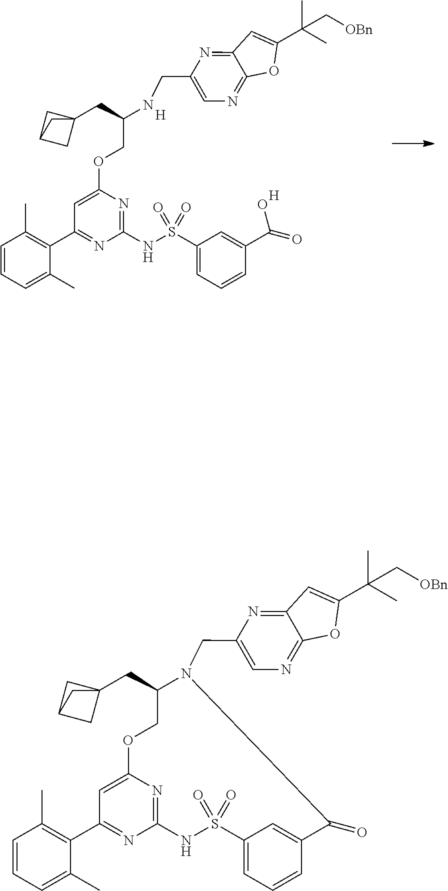

3-[[4-[(2R)-2-[[6-(2-benzyloxy-1,1-dimethyl-ethyl)furo[2,3-b]pyrazin-2-yl]methylamino]-3-(1-bicyclo[1.1.1]pentanyl)propoxy]-6-(2,6-dimethylphenyl)pyrimidin-2-yl]sulfamoyl]benzoic acid (hydrochloride salt) (66.8 mg, 0.07827 mmol) was combined under nitrogen with CDMT (37.5 mg, 0.2136 mmol) and DMF (2.5 mL). The solution was stirred at 0° C. 4-Methyl-morpholine (60 μL, 0.5457 mmol) was added and the mixture was stirred in the cooling bath that was allowed to warm to room temperature. After 16 h, the reaction was filtered and purified by reverse phase HPLC (30-99% acetonitrile/5 mM aqueous HCl over 30 min) to give as a white solid (11R)-12-[[6-(2-benzyloxy-1,1-dimethyl-ethyl)furo[2,3-b]pyrazin-2-yl]methyl]-11-(1-bicyclo[1.1.1]pentanylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (43.3 mg, 69%), ¹H NMR (400 MHz, Chloroform-d) δ 9.22-8.70 (m, 2H), 8.42 (s, 1H), 8.14 (d, J=7.9 Hz, 1H), 7.89 (dt, J=7.9, 1.3 Hz, 1H), 7.67 (t, J=7.8 Hz, 1H), 7.32-7.26 (m, 3H), 7.26-7.22 (m, 2H), 7.22-7.18 (m, 1H), 7.04 (d, J=7.6 Hz, 2H), 6.71 (s, 1H), 6.21 (s, 1H), 5.39 (dd, J=11.4, 4.0 Hz, 1H), 5.33 (d, J=15.0 Hz, 1H), 4.51 (s, 2H), 4.28-4.18 (m, 2H), 4.11-4.00 (m, 1H), 3.64 (d, J=8.9 Hz, 1H), 3.59 (d, J=8.9 Hz, 1H), 2.44 (s, 1H), 2.08-1.94 (m, 7H), 1.81 (dd, J=15.6, 3.2 Hz, 1H), 1.59-1.54 (m, 6H), 1.43 (d, J=5.4 Hz, 6H). ESI-MS m/z calc. 798.31995, found 799.4 (M+1)⁺; Retention time: 2.02 minutes. LC method C.

### Step 7: (11R)-11-(1-Bicyclo[1.1.1]pentanylmethyl)-6-(2,6-dimethylphenyl)-12-[[6-(2-hydroxy-1,1-dimethyl-ethyl)furo[2,3-b]pyrazin-2-yl]methyl]-2,2-dioxo-9-oxa-2λ⁶-thia-3,5,12,19-tetrazatricyclo[12.3.1.14,8]nonadeca-1(18),4(19),5,7,4,16-hexaen-13-one (Compound I-113

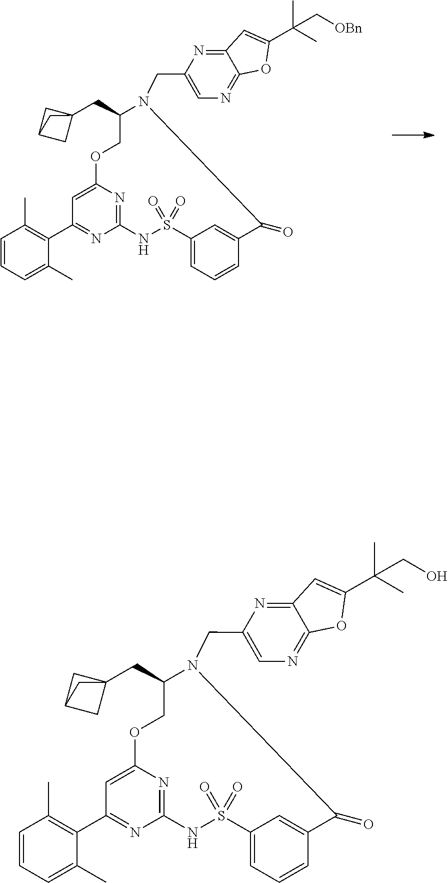

A nitrogen flushed solution of (11R)-12-[[6-(2-benzyloxy-1,1-dimethyl-ethyl)furo[2,3-b]pyrazin-2-yl]methyl]-11-(1-bicyclo[1.1.1]pentanylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (22.3 mg, 0.02763 mmol) in MeOH (500 μL) was stirred at room temperature. The mixture was a suspension, added EtOAc (500 μL), followed by Pd/C (2.5 mg of 10% w/w, 0.002349 mmol). The mixture was evacuated and then stirred under a hydrogen atmosphere using a hydrogen-filled balloon at room temperature for 3 hours. The crude material was then filtered and concentrated. Then the solid was dissolved in DMSO and was purified by reverse phase HPLC preparative chromatography (1-99% acetonitrile/5 mM aqueous HCl over 30 min) to provide a white solid (11R)-11-(1-bicyclo[1.1.1]pentanylmethyl)-6-(2,6-dimethylphenyl)-12-[[6-(2-hydroxy-1,1-dimethyl-ethyl)furo[2,3-b]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (17.5 mg, 88%), ¹H NMR (400 MHz, Chloroform-d) δ 8.70 (t, J=1.9 Hz, 1H), 8.43 (s, 1H), 8.16 (d, J=8.1 Hz, 1H), 7.92 (dt, J=7.8, 1.4 Hz, 1H), 7.70 (t, J=7.8 Hz, 1H), 7.23 (d, J=7.6 Hz, 1H), 7.09 (d, J=7.6 Hz, 2H), 6.75 (s, 1H), 6.30 (s, 1H), 5.45 (dd, J=11.5, 4.0 Hz, 1H), 5.33-5.30 (m, 1H), 4.33 (t, J=11.5 Hz, 1H), 4.23 (d, J=14.9 Hz, 1H), 4.10-3.98 (m, 1H), 3.85-3.75 (m, 2H), 2.46 (s, 1H), 2.09 (s, 6H), 2.04-1.97 (m, 1H), 1.80 (dd, J=15.6, 3.2 Hz, 1H), 1.62-1.52 (m, 6H), 1.43 (d, J=3.0 Hz, 6H) plus 2 exchangeable protons not observed in CDCl₃ ¹H NMR, likely the sulfonamide NH and the OH. ESI-MS m/z calc. 708.273, found 709.4 (M+1)⁺; Retention time: 1.98 minutes. LC method A.

### Example 65: Preparation of Compound III-16

### Step 1: Methyl 3-methyl-1-oxo-pyrazine-2-carboxylate and methyl 3-methyl-4-oxo-pyrazine-2-carboxylate

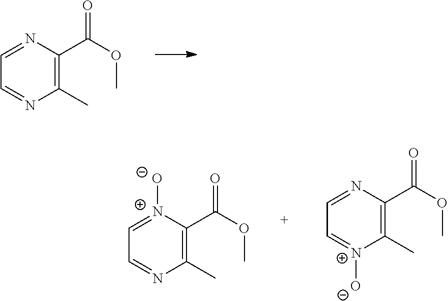

A solution of methyl 3-methylpyrazine-2-carboxylate (9.1 g, 59.809 mmol) in DCM (100 mL) was cooled to 0° C. and urea hydrogen peroxide adduct was added (7.8 g, 82.917 mmol), followed by dropwise addition of trifluoracetic acid anhydride (16.319 g, 10.8 mL, 77.698 mmol). The resulting mixture was stirred at 0° C. for 1 h, and at rt for 18 h. The reaction was diluted with DCM (100 mL) and quenched with a saturated Na₂SO₃ solution (50 mL). The aqueous layer was back extracted with DCM (2×50 mL). The combined organic extracts were dried (MgSO₄), filtered and concentrated in vacuo. Purification (20-80% EtOAc/hexanes) afforded two fractions: Methyl 3-methyl-1-oxo-pyrazine-2-carboxylate (2.9 g, 26%). ¹H NMR (500 MHz, CDCl₃) δ 8.38 (d, J=3.8 Hz, 1H), 8.24 (d, J=3.9 Hz, 1H), 4.02 (s, 3H), 2.70 (s, 3H). ESI-MS m/z calc. 168.0535, found 169.1 (M+1)⁺; Retention time: 1.67 minutes, and methyl 3-methyl-4-oxo-pyrazine-2-carboxylate (1.7 g, 8%) ESI-MS m/z calc. 168.0535, found 169.3 (M+1)⁺; Retention time: 1.69 minutes (mixed with the previous isomer, 1:1 ratio), LC method D. The mixture of two Regio isomers (1.7 g fraction), containing 3-(methoxycarbonyl)-2-methylpyrazine 1-oxide and 2-(methoxycarbonyl)-3-methylpyrazine 1-oxide was taken to the next step without further purification.

### Step 2: Methyl 6-chloro-3-methyl-pyrazine-2-carboxylate and methyl 5-chloro-3-methyl-pyrazine-2-carboxylate

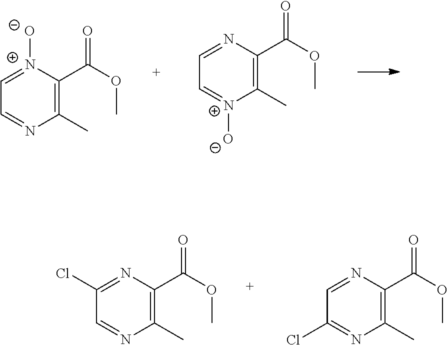

A solution of the mixture of methyl 3-methyl-1-oxo-pyrazine-2-carboxylate (850 mg, 5.0550 mmol) and methyl 3-methyl-4-oxo-pyrazine-2-carboxylate (850 mg, 5.0550 mmol) in toluene (20 mL) was cooled to 0° C. and phosphorus oxychloride (1 mL, 30.3 mmol) was added under nitrogen followed by DMF (94.400 mg, 0.1 mL, 1.2915 mmol). The reaction mixture was stirred at rt for 4 h and then heated to 65° C. for 6 h. Toluene was evaporated, and to the residue was treated with phosphorus oxychloride (8.2250 g, 5 mL, 53.642 mmol) and DMF (188.80 mg, 0.2 mL, 2.5830 mmol). The resulting mixture was stirred at room temperature for 12 hours, then at 65° C. for 6 hours until completion of the conversion. Then the reaction mixture was concentrated to dryness and reevaporated with 10 ml of toluene, cooled to rt, diluted with EtOAc (200 mL) and washed with saturated NaHCO₃ solution (200 mL). The aqueous layer was back extracted with EtOAc (2×50 mL). The combined organic extracts were dried (MgSO₄), filtered and concentrated in vacuo. The crude was purified by flash chromatography (80 g silica gel column, 0-20% EtOAc/hexanes) to give two separated isomers, methyl 6-chloro-3-methyl-pyrazine-2-carboxylate (730 mg, 77%) (first product to elute), ESI-MS m/z calc. 186.0196, found 187.6 (M+1)⁺; Retention time: 3.29 minutes; ¹H NMR (500 MHz, DMSO) δ 8.88 (s, 1H), 3.90 (s, 5H), 2.71 (s, 5H), and methyl 5-chloro-3-methyl-pyrazine-2-carboxylate (300 mg, 32%) (second product to elute), ESI-MS m/z calc. 186.0196, found 187.6 (M+1)⁺; Retention time: 3.33 minutes; ¹H NMR (500 MHz, DMSO) δ 8.73 (s, 1H), 3.90 (s, 5H), 2.70 (s, 5H). LC method D.

### Step 3: Methyl 3-(bromomethyl)-5-chloro-pyrazine-2-carboxylate

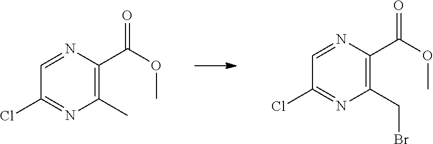

A mixture of methyl 5-chloro-3-methyl-pyrazine-2-carboxylate (1 g, 5.3592 mmol), NBS (1.24 g, 6.9669 mmol) and 2,2′-Azobis(2-methylpropionitrile) (130 mg, 0.7917 mmol) in trifluorotoluene (10 mL) was refluxed for 3 h. An additional amount of NBS (990 mg, 5.5623 mmol) was added and the mixture was refluxed another 2 h. The reaction was cooled to rt and filtered through zeolite, the precipitate was washed with DCM (2×20 mL). The combined filtrate was evaporated, and the residue was purified by silica-gel chromatography (0-10% Ethyl acetate in hexane), to give methyl 3-(bromomethyl)-5-chloro-pyrazine-2-carboxylate (1.48 g, 42%). ESI-MS m/z calc. 263.9301, found 267.3 (M+1)⁺; Retention time: 4.26 minutes; LC method D.

### Step 4: 3-Chloro-6-isopropyl-5H-pyrrolo[3,4-b]pyrazin-7-one

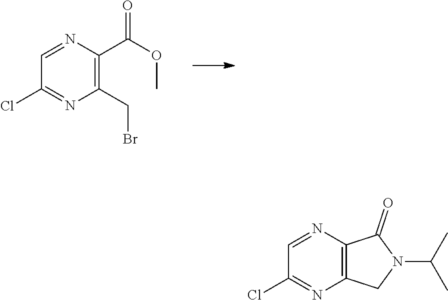

To a solution of methyl 3-(bromomethyl)-5-chloro-pyrazine-2-carboxylate (4.6 g, 17.326 mmol) and isopropyl amine (1.15 g, 19.455 mmol) in acetonitrile (50 mL), NaHCO₃ (3 g, 35.711 mmol) was added, and the suspension was stirred at room temperature for 12 hours. The reaction was quenched with ethyl acetate (200 mL) and water (20 mL). The layers were separated, and the aqueous phase was extracted with ethyl acetate (2×50 mL). The organic extracts were combined, dried over magnesium sulfate, filtered, and evaporated. The residue was purified by silica gel chromatography (0-50% ethyl acetate in hexane) to give 3-chloro-6-isopropyl-5H-pyrrolo[3,4-b]pyrazin-7-one (1.6 g, 39%) as of light-yellow solid. ESI-MS m z calc. 211.05124, found 212.4 (M+1)⁺; Retention time: 3.03 minutes; LC method D.

### Step 5: 6-Isopropyl-3-vinyl-5H-pyrrolo[3,4-b]pyrazin-7-one

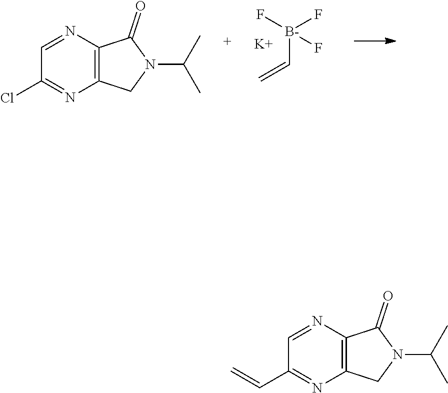

A mixture of 3-chloro-6-isopropyl-5H-pyrrolo[3,4-b]pyrazin-7-one (1.5 g, 7.0872 mmol), potassium vinyltrifluoroborate (2.4 g, 17.917 mmol), Pd(dppf)Cl₂ (410 mg, 0.5021 mmol) and TEA (3.2670 g, 4.5 mL, 32.286 mmol) in EtOH (150 mL) was degassed and heated in a sealed vial for 2 hours at 90° C. The reaction mixture was evaporated. The residue was purified by silica gel chromatography to give 6-isopropyl-3-vinyl-5H-pyrrolo[3,4-b]pyrazin-7-one (1230 mg, 68%). ESI-MS m/z calc. 203.10587, found 204.3 (M+1)⁺; Retention time: 3.57 minutes; LC method E.

### Step 6: 6-Isopropyl-7-oxo-5H-pyrrolo[3,4-b]pyrazine-3-carbaldehyde

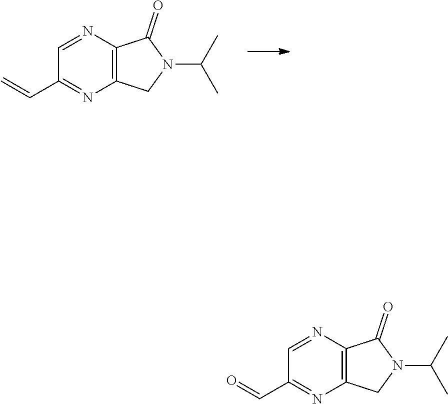

To a solution of 6-isopropyl-3-vinyl-5H-pyrrolo[3,4-b]pyrazin-7-one (1.23 g, 6.0520 mmol) in dioxane (60 mL) and H₂O (3 mL) was added pyridine (1.9560 g, 2 mL, 24.728 mmol), OsO₄ in tBuOH (5 mL of 2.5% w/v, 0.4917 mmol) then NaIO₄ (5 g, 23.376 mmol) at room temperature. The reaction was stirred at room temperature overnight. The reaction was diluted with DCM (30 mL) washed with sodium bicarbonate (50 mL), extracted with DCM (2×10 mL), washed with brine (20 mL) dried over sodium sulfate filtered and concentrated in vacuo. The residue was purified by flash chromatography (loaded onto a 12 g silica cartridge with DCM, and eluted with 50-100% EtOAc in hexanes over a 15 min gradient) to provide 6-isopropyl-7-oxo-5H-pyrrolo[3,4-b]pyrazine-3-carbaldehyde (289 mg, 23%) as a colorless solid. ¹H NMR (500 MHz, CDCl₃) δ 10.21 (s, 1H), 9.32 (s, 1H), 4.89-4.80 (m, 1H), 4.53 (s, 2H), 1.38 (d, J=3.1 Hz, 6H). ESI-MS m/z calc. 205.08513, found 205.9 (M+1)⁺; Retention time: 0.67 minutes; LC method H.

### Step 7: 3-[[4-[(2R)-3-(1-Bicyclo[1.1.1]pentanyl)-2-[(6-isopropyl-7-oxo-5H-pyrrolo[3,4-b]pyrazin-3-yl)methylamino]propoxy]-6-(2,6-dimethylphenyl)pyrimidin-2-yl]sulfamoyl]benzoic acid

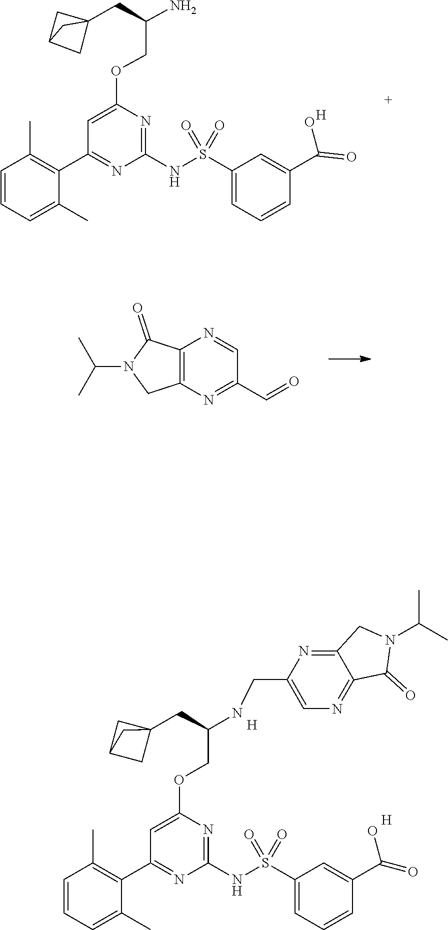

A 4 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (80 mg, 0.1431 mmol), 6-isopropyl-7-oxo-5H-pyrrolo[3,4-b]pyrazine-3-carbaldehyde (29.7 mg, 0.1447 mmol), anhydrous DCM (0.65 mL), and acetic acid (0.0084 mL, 0.1477 mmol). The mixture was cooled down in an ice bath. DIEA (0.06 mL, 0.3445 mmol) was added, followed by sodium triacetoxyborohydride (188.6 mg, 0.8899 mmol), and the reaction was vigorously stirred at 0° C. for 6 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-[(2R)-3-(1-bicyclo[1.1.1]pentanyl)-2-[(6-isopropyl-7-oxo-5H-pyrrolo[3,4-b]pyrazin-3-yl)methylamino]propoxy]-6-(2,6-dimethylphenyl)pyrimidin-2-yl]sulfamoyl]benzoic acid (hydrochloride salt) (50.0 mg, 45%) as a white solid. ESI-MS m/z calc. 711.2839, found 712.4 (M+1)⁺; Retention time: 1.31 minutes. LC method A.

### Step 8: (11R)-11-(1-Bicyclo[1.1.1]pentanylmethyl)-6-(2,6-dimethylphenyl)-12-[(6-isopropyl-7-oxo-5H-pyrrolo[3,4-b]pyrazin-3-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one (Compound III-16

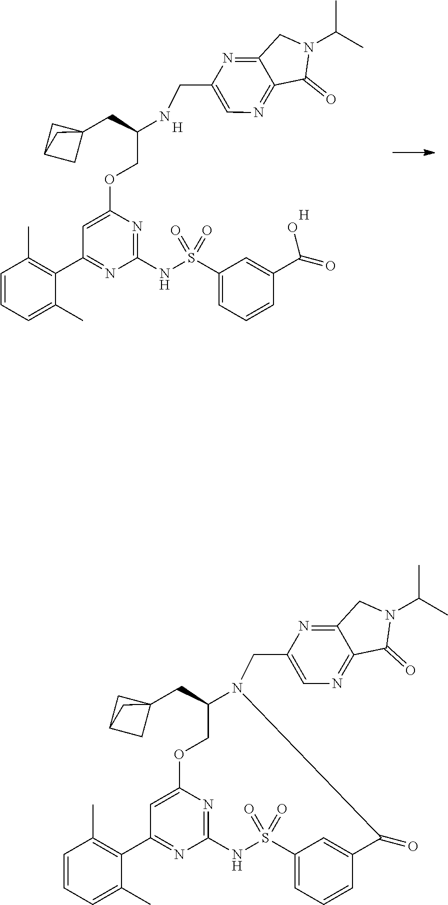

3-[[4-[(2R)-3-(1-bicyclo[1.1.1]pentanyl)-2-[(6-isopropyl-7-oxo-5H-pyrrolo[3,4-b]pyrazin-3-yl)methylamino]propoxy]-6-(2,6-dimethylphenyl)pyrimidin-2-yl]sulfamoyl]benzoic acid (hydrochloride salt) (50.0 mg, 0.06481 mmol) was combined with CDMT (17.0 mg, 0.09683 mmol) in DMF (5 mL) and cooled to 0° C. N-methylmorpholine (21.4 μL, 0.1946 mmol) was added by syringe and the reaction was stirred at 0° C. for 30 minutes. The ice bath was then removed and stirring was continued for an additional 16 hours at room temperature. The reaction mixture was then partitioned between 50 mL 1 M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-11-(1-bicyclo[1.1.1]pentanylmethyl)-6-(2,6-dimethylphenyl)-12-[(6-isopropyl-7-oxo-5H-pyrrolo[3,4-b]pyrazin-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (17.8 mg, 39%) as a white solid. ¹H NMR (400 MHz, CDCl₃) δ 8.94 (s, 1H), 8.80 (t, J=1.8 Hz, 1H), 8.10 (d, J=7.9 Hz, 1H), 7.86 (dt, J=7.7, 1.4 Hz, 1H), 7.66 (t, J=7.8 Hz, 1H), 7.21 (t, J=7.6 Hz, 1H), 7.04 (d, J=7.6 Hz, 2H), 6.19 (s, 1H), 5.61-5.52 (m, 1H), 5.26 (d, J=15.7 Hz, 1H), 4.82 (hept, J=6.8 Hz, 1H), 4.46 (s, 2H), 4.26 (d, J=15.7 Hz, 1H), 4.11-4.02 (m, 2H), 2.47 (s, 1H), 2.01 (s, 6H), 1.95-1.79 (m, 2H), 1.64-1.54 (m, 6H), 1.34 (d, J=4.5 Hz, 3H), 1.33 (d, J=4.5 Hz, 3H). ESI-MS m/z calc. 693.2733, found 694.7 (M+1)⁺; Retention time: 1.76 minutes. LC method A.

### Example 66: Preparation of Compound I-117

### Step 1: Methyl 6-isopropyl-5H-pyrrolo[2,3-b]pyrazine-3-carboxylate

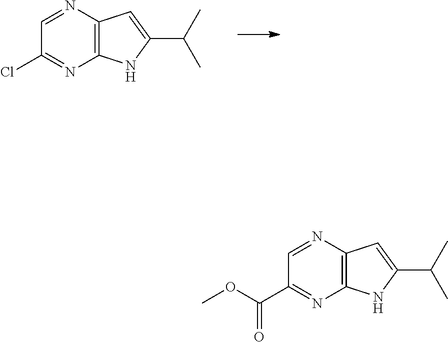 [SMILES: CC(C)c1cc2ncc(Cl)nc2[nH]1 ; COC(=O)c1cnc2cc(C(C)C)[nH]c2n1]

1,1′-Bis(diphenylphosphino)ferrocene palladium(II) chloride, complex with dichloromethane (300 mg, 0.3674 mmol) was added to a suspension of 3-chloro-6-isopropyl-5H-pyrrolo[2,3-b]pyrazine (1.38 g, 7.0464 mmol), triethylamine (2.9040 g, 4 mL, 28.698 mmol) in anhydrous methanol (16 mL). Nitrogen gas was injected into the suspension for 1 min. The mixture was then stirred at 100° C. under 50 psi carbon monoxide pressure for 18 hours. The reaction mixture was left to cool down to room temperature and then DCM (50 mL) was added. The mixture was filtered on Celite and the pad was washed with DCM (2×75 mL). The filtrate was concentrated under reduced pressure and the resulting residue was purified on silica gel using 0 to 80% ethylacetate in heptanes to provide methyl 6-isopropyl-5H-pyrrolo[2,3-b]pyrazine-3-carboxylate (180 mg, 12%) as a yellow fluffy powder ESI-MS m/z calc. 219.1008, found 220.2 (M+1)⁺; Retention time: 1.5 minutes and another fraction of pure methyl 6-isopropyl-5H-pyrrolo[2,3-b]pyrazine-3-carboxylate (1.146 g, 74%) as a pale yellow powder ESI-MS m/z calc. 219.10078, found 220.2 (M+1)⁺; Retention time: 1.5 minutes; LC method I.

### Step 2: (6-Isopropyl-5H-pyrrolo[2,3-b]pyrazin-3-yl)methanol

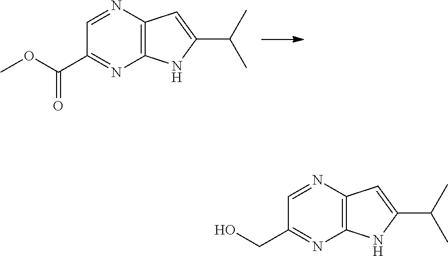 [SMILES: COC(=O)c1cnc2cc(C(C)C)[nH]c2n1 ; CC(C)c1cc2ncc(CO)nc2[nH]1]

A solution of methyl 6-isopropyl-5H-pyrrolo[2,3-b]pyrazine-3-carboxylate (1.14 g, 5.1946 mmol) in THE (60 mL) was cooled to 0° C. then slowly added to a suspension of LAH (500 mg, 13.174 mmol) in THE (60 mL) maintained at 0° C. The resulting suspension was left stirring at this temperature for 1 h. Water (1.2 ml) was added followed by 15% aqueous NaOH (1.2 mL) and the mixture was stirred for 15 min at room temperature. Magnesium sulfate (3 g) and solid ammonium chloride (3 g) were added, and the reaction mixture was left stirring for 1 h then filtered on Celite. The cake was washed with Me-THF (2×50 mL) and the combined filtrates were then concentrated under reduced pressure to provide (6-isopropyl-5H-pyrrolo[2,3-b]pyrazin-3-yl)methanol (1.01 g, 98%) as a yellow solid ESI-MS m/z calc. 191.1059, found 192.2 (M+1)⁺; Retention time: 1.21 minutes that was pure enough to be used directly in the next reaction without further purification. LC method I.

### Step 3: 6-Isopropyl-5H-pyrrolo[2,3-b]pyrazine-3-carbaldehyde

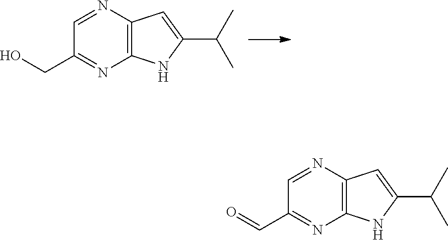

Manganese dioxide (activated) (4.2 g, 48.311 mmol) was added to a solution of (6-isopropyl-5H-pyrrolo[2,3-b]pyrazin-3-yl)methanol (500 mg, 2.4316 mmol) in THE (15 mL) and left stirring at room temperature for 1 h. The reaction mixture was filtered on a Celite pad and the cake was washed with Me-THF (100 mL×3). The filtrates was concentrated under reduced pressure and the resulting residue was triturated in heptanes, filtered, washed with heptanes and dried under high vacuum to provide 6-isopropyl-5H-pyrrolo[2,3-b]pyrazine-3-carbaldehyde (311 mg, 66%) as a yellow powder ¹H NMR (400 MHz, DMSO-d6) δ 12.49 (br. s, 1H), 10.03 (s, 1H), 8.88 (s, 1H), 6.57 (s, 1H), 3.19 (spt, J=6.8 Hz, 1H), 1.36 (d, J=6.8 Hz, 6H). ESI-MS m z calc. 189.09021, found 190.1 (M+1)⁺; Retention time: 2.98 minutes; LC method J.

### Step 4: 6-Isopropyl-5-(2-methoxyethyl)pyrrolo[2,3-b]pyrazine-3-carbaldehyde

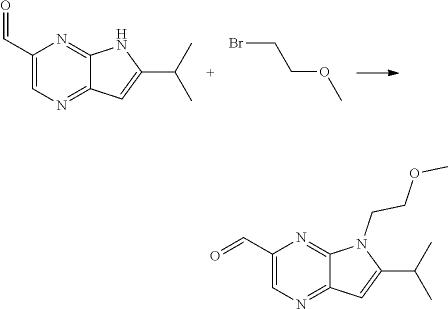

To a stirred solution of 6-isopropylpyrrolo[2,3-b]pyrazine-3-carbaldehyde (38 mg, 0.2008 mmol) in anhydrous DMF (1 mL) was added cesium carbonate (135 mg, 0.4143 mmol) in one portion. The heterogeneous yellow mixture was stirred at ambient temperature under nitrogen for 10 min. Then 1-bromo-2-methoxy-ethane (30 mg, 0.2158 mmol) was added and stirring continued overnight (18 h). The reaction was quenched with aqueous 1 M HCl (1.0 mL) and extracted with ethyl acetate (3×10 mL). washed with brine (10 mL), dried over sodium sulfate, filtered, and concentrated under reduced pressure. Purification of the crude mixture by preparative reverse-phase HPLC (1-99% acetonitrile in water over 15 min, 5 mM HCl as modifier) furnished product as a brown gum. 6-isopropyl-5-(2-methoxyethyl)pyrrolo[2,3-b]pyrazine-3-carbaldehyde (27 mg, 54%) ¹H NMR (400 MHz, CDCl₃) δ 10.19 (s, 1H), 8.95 (s, 1H), 7.13 (s, 1H), 4.69 (t, J=5.1 Hz, 2H), 3.79 (t, J=5.1 Hz, 2H), 3.57-3.49 (m, 1H), 3.29 (s, 3H), 1.45 (d, J=6.8 Hz, 6H). ESI-MS m/z calc. 247.13208, found 248.4 (M+1)⁺; Retention time: 1.3 minutes. LC method A.

### Step 5: 3-[[4-(2,6-Dimethylphenyl)-6-[(2R)-2-[[6-isopropyl-5-(2-methoxyethyl)pyrrolo[2,3-b]pyrazin-3-yl]methylamino]-4,4-dimethyl-pentoxy]pyrimidin-2-yl]sulfamoyl]benzoic acid

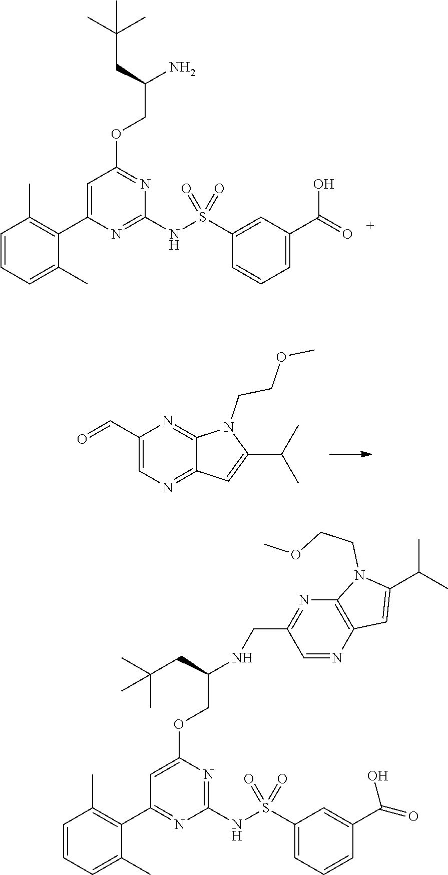 [SMILES: Cc1cccc(C)c1-c1cc(OC[C@H](N)CC(C)(C)C)nc(NS(=O)(=O)c2cccc(C(=O)O)c2)n1 ; COCCn1c(C(C)C)cc2ncc(C=O)nc21 ; COCCn1c(C(C)C)cc2ncc(CN[C@@H](COc3cc(-c4c(C)cccc4C)nc(NS(=O)(=O)c4cccc(C(=O)O)c4)n3)CC(C)(C)C)nc21]

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (59 mg, 0.1075 mmol) and 6-isopropyl-5-(2-methoxyethyl)pyrrolo[2,3-b]pyrazine-3-carbaldehyde (27 mg, 0.1092 mmol) in anhydrous dichloromethane (400 μL) were added glacial acetic acid (10 μL, 0.1758 mmol) and DIPEA (55 μL, 0.3158 mmol), in that order, at 0° C. under nitrogen. After 2 min, sodium triacetoxyborohydride (80 mg, 0.3775 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish product as a yellow solid. 3-[[4-(2,6-dimethylphenyl)-6-[(2R)-2-[[6-isopropyl-5-(2-methoxyethyl)pyrrolo[2,3-b]pyrazin-3-yl]methylamino]-4,4-dimethyl-pentoxy]pyrimidin-2-yl]sulfamoyl]benzoic acid (hydrochloride salt) (53 mg, 63%) ESI-MS m/z calc. 743.3465, found 745.0 (M+1)⁺; Retention time: 1.53 minutes. LC method A.

### Step 6: (11R)-6-(2,6-Dimethylphenyl)-11-(2,2-dimethylpropyl)-12-[[6-isopropyl-5-(2-methoxyethyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-117

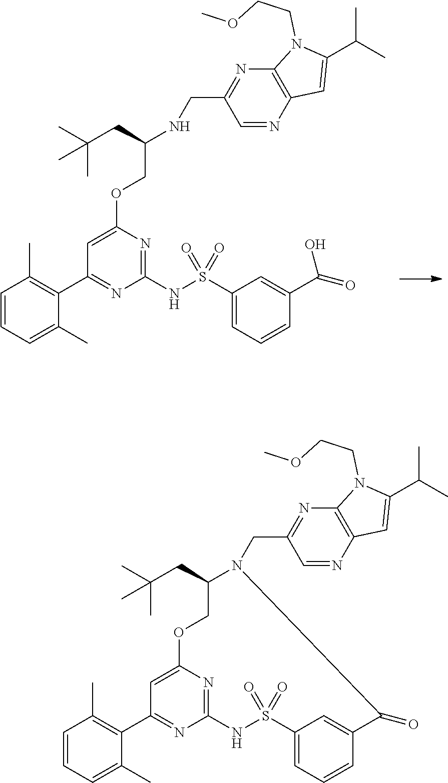 [SMILES: COCCn1c(C(C)C)cc2ncc(CN[C@@H](COc3cc(-c4c(C)cccc4C)nc(NS(=O)(=O)c4cccc(C(=O)O)c4)n3)CC(C)(C)C)nc21 ; COCCn1c(C(C)C)cc2ncc(CN3C(=O)c4cccc(c4)S(=O)(=O)Nc4nc(cc(-c5c(C)cccc5C)n4)OC[C@H]3CC(C)(C)C)nc21]

To a stirred solution of 3-[[4-(2,6-dimethylphenyl)-6-[(2R)-2-[[6-isopropyl-5-(2-methoxyethyl)pyrrolo[2,3-b]pyrazin-3-yl]methylamino]-4,4-dimethyl-pentoxy]pyrimidin-2-yl]sulfamoyl]benzoic acid (hydrochloride salt) (53 mg, 0.06792 mmol) in anhydrous DMF (2.4 mL) was added 2-chloro-4,6-dimethoxy-1,3,5-triazine (18 mg, 0.1025 mmol) (CDMT), followed by addition of 4-methylmorpholine (50 μL, 0.4548 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished product in two fractions, both as a yellow solid. First fraction (11R)-6-(2,6-dimethylphenyl)-11-(2,2-dimethylpropyl)-12-[[6-isopropyl-5-(2-methoxyethyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (27 mg, 54%) ¹H NMR (400 MHz, CDCl₃) δ 8.94 (t, J=1.8 Hz, 1H), 8.49 (s, 1H), 8.05 (dt, J=8.1, 1.4 Hz, 1H), 7.88 (dt, J=7.8, 1.3 Hz, 1H), 7.65 (t, J=7.8 Hz, 1H), 7.22 (t, J=7.6 Hz, 1H), 7.07 (d, J=7.6 Hz, 2H), 6.48 (s, 1H), 6.27 (s, 1H), 5.55 (dd, J=11.2, 4.3 Hz, 1H), 5.36 (d, J=15.7 Hz, 1H), 4.59 (dt, J=14.8, 4.9 Hz, 1H), 4.50 (ddd, J=14.8, 7.0, 4.3 Hz, 1H), 4.39 (d, J=15.7 Hz, 1H), 4.31-4.21 (m, 1H), 4.08 (t, J=11.4 Hz, 1H), 3.81-3.72 (m, 1H), 3.71-3.64 (m, 1H), 3.30 (hept, J=7.0 Hz, 1H), 3.13 (s, 3H), 2.02 (s, 6H), 1.77 (dd, J=15.1, 8.8 Hz, 1H), 1.59 (dd, J=15.2, 1.7 Hz, 1H), 1.37 (d, J=6.8 Hz, 3H), 1.36 (d, J=6.8 Hz, 3H), 0.65 (s, 9H). ESI-MS m/z calc. 725.33594, found 726.7 (M+1)⁺; Retention time: 1.9 minutes. and the second fraction (11R)-6-(2,6-dimethylphenyl)-11-(2,2-dimethylpropyl)-12-[[6-isopropyl-5-(2-methoxyethyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (8 mg, 15%) ESI-MS m/z calc. 725.33594, found 726.8 (M+1)⁺; Retention time: 1.87 minutes. LC method A.

### Example 67: Preparation of Compound I-121

### Step 1: Methyl (2R)-2-(benzyloxycarbonylamino)-3-(cyclopentoxy)propanoate

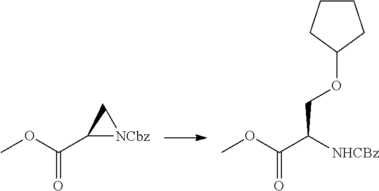

To a solution of O1-benzyl O2-methyl (2R)-aziridine-1,2-dicarboxylate (25.1 g, 96.031 mmol) and cyclopentanol (18.980 g, 20 mL, 220.36 mmol) in CHCl₃ (200 mL) at 0° C. was added dropwise boron trifluoride diethyl etherate (2.7600 g, 2.4000 mL, 19.446 mmol). The reaction mixture was stirred at 0° C. for 30 minutes then at room temperature for 2 hours. To the reaction mixture was added an aqueous saturated solution of sodium bicarbonate (100 mL) and the solvent was removed under reduced pressure. The aqueous solution was diluted with EtOAc (200 mL) and the phases were separated. The organic layer was washed with brine (2×200 mL), dried over anhydrous sodium sulfate, filtered and concentrated under reduced pressure. The crude solution was first purified by reversed-phase chromatography (Column: C₁₈. Eluent: 5-100% methanol in water). The fractions containing the product were combined and most of the methanol was removed under reduced pressure. The aqueous solution was diluted with EtOAc (200 mL) and the organic layer was washed with brine (2×100 mL), dried over anhydrous sodium sulfate, filtered and concentrated under reduced pressure. Purification by silica gel chromatography (Gradient: 0-10% EtOAc in heptane) afforded methyl (2R)-2-(benzyloxycarbonylamino)-3-(cyclopentoxy)propanoate (12.9 g, 42%) as a pale yellow oil. ¹H NMR (400 MHz, CDCl₃) δ 7.43-7.30 (m, 5H), 5.59 (d, J=8.6 Hz, 1H), 5.20-5.08 (m, 2H), 4.47 (dt, J=8.8, 3.1 Hz, 1H), 3.89-3.79 (m, 2H), 3.76 (s, 3H), 3.61 (dd, J=9.3, 3.2 Hz, 1H), 1.73-1.54 (m, 6H, overlapped with water), 1.53-1.43 (m, 2H). ESI-MS m/z calc. 321.1576, found 322.2 (M+1)⁺; Retention time: 1.89 minutes. Mixed fractions were combined and purified by reversed-phase chromatography (Column: C₁₈. Gradient: 20-70% methanol in water) to afford methyl (2R)-2-(benzyloxycarbonylamino)-3-(cyclopentoxy)propanoate (11 g, 35%) as a pale yellow oil. ¹H NMR (400 MHz, CDCl₃) δ 7.42-7.30 (m, 5H), 5.59 (d, J=8.3 Hz, 1H), 5.20-5.08 (m, 2H), 4.47 (dt, J=8.7, 3.1 Hz, 1H), 3.89-3.80 (m, 2H), 3.76 (s, 3H), 3.61 (dd, J=9.3, 3.2 Hz, 1H), 1.70-1.55 (m, 6H, overlapped with water), 1.53-1.44 (m, 2H). ESI-MS m/z calc. 321.1576, found 322.2 (M+1)⁺; Retention time: 1.89 minutes. Purification also afforded methyl (2S)-3-(benzyloxycarbonylamino)-2-(cyclopentoxy)propanoate (3.14 g, 10%) as a pale yellow oil. ¹H NMR (400 MHz, CDCl₃) δ 7.42-7.29 (m, 5H), 5.19-5.05 (m, 3H), 4.08-3.99 (m, 2H), 3.74 (s, 3H), 3.67-3.54 (m, 1H), 3.44-3.33 (m, 1H), 1.82-1.46 (m, 8H, overlapped with water). ESI-MS m/z calc. 321.1576, found 322.2 (M+1)⁺; Retention time: 1.83 minutes. LC method I.

### Step 2: Benzyl N-[(1S)-1-(cyclopentoxymethyl)-2-hydroxy-ethyl]carbamate

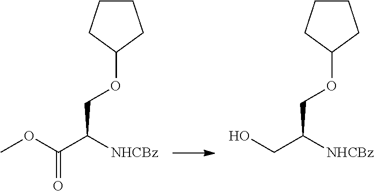

To a solution of methyl (2R)-2-(benzyloxycarbonylamino)-3-(cyclopentoxy)propanoate (1.003 g, 3.1179 mmol) in THE (10 mL) and methanol (50 L) at 0° C. was added lithium borohydride (208 mg, 9.5484 mmol). The reaction was stirred for 4 hours then the reaction was diluted with EtOAc (10 mL). To the biphasic solution was slowly added an aqueous solution of sodium bicarbonate (10 mL) in order to keep the temperature below 10° C. The reaction mixture was extracted with EtOAc (3×20 mL). The combined organic layers were washed with an aqueous saturated solution of sodium bicarbonate (2×20 mL) and brine (20 mL). The organic layer was dried over anhydrous sodium sulfate, filtered and concentrated under reduced pressure to afford benzyl N-[(1S)-1-(cyclopentoxymethyl)-2-hydroxy-ethyl]carbamate (902 mg, 99%) as a pale yellow oil. ¹H NMR (400 MHz, CDCl₃) δ 7.41-7.30 (m, 5H), 5.52-5.41 (m, 1H), 5.12 (s, 2H), 3.93-3.78 (m, 3H), 3.77-3.66 (m, 1H), 3.66-3.53 (m, 2H), 2.70 (d, J=7.1 Hz, 1H), 1.79-1.48 (m, 8H). ESI-MS m/z calc. 293.16272, found 294.2 (M+1)⁺; Retention time: 1.73 minutes; LC method I.

### Step 3: (2S)-2-Amino-3-(cyclopentoxy)propan-1-ol

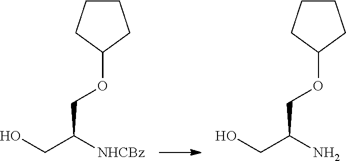

A solution of benzyl N-[(1S)-1-(cyclopentoxymethyl)-2-hydroxy-ethyl]carbamate (2.207 g, 7.2148 mmol) in methanol (20 mL) was sparged with nitrogen for 10 minutes then palladium on carbon (772 mg, 10% w/w, 0.7254 mmol) was added. The suspension was then sparged with hydrogen for 5 minutes then stirred under a hydrogen atmosphere for 25 hours. The suspension was sparged with nitrogen for 10 minutes then filtered through Celite, washing with MeOH (2×100 mL). The solution was concentrated in vacuo and the residue was diluted with diethyl ether (20 mL). To the solution at 0° C. was added dropwise a solution of hydrogen chloride in diethyl ether (7.5 mL of 2 M, 15.000 mmol). To the reaction mixture was added heptane (20 mL) and the suspension was stirred at 0° C. for 15 minutes. The solid was filtered and washed with heptane (2×20 mL) to afford (2S)-2-amino-3-(cyclopentoxy)propan-1-ol (hydrochloride salt) (1.27 g, 85%) as a pale grey solid that was used in the following Step without further purification. ¹H NMR (400 MHz, DMSO-d6) δ 7.97 (br. s., 3H), 5.32-5.21 (m, 1H), 3.94-3.86 (m, 1H), 3.63-3.54 (m, 1H), 3.54-3.46 (m, 2H), 3.46-3.39 (m, 1H), 3.22-3.12 (m, 1H), 1.72-1.55 (m, 6H), 1.53-1.41 (m, 2H). ESI-MS m/z calc. 159.12593, found 160.2 (M+1)⁺; Retention time: 0.68 minutes; LC method I.

### Step 4: 3-[[4-[(2R)-2-Amino-3-(cyclopentoxy)propoxy]-6-(2,6-dimethylphenyl)pyrimidin-2-yl]sulfamoyl]benzoic acid

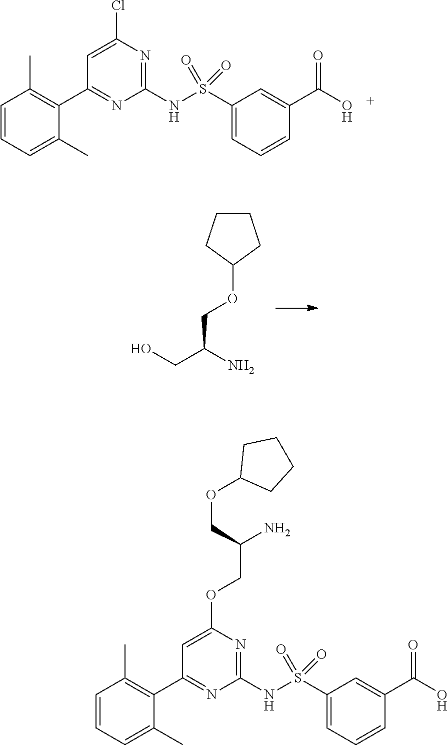 [SMILES: Cc1cccc(C)c1-c1cc(Cl)nc(NS(=O)(=O)c2cccc(C(=O)O)c2)n1 ; N[C@@H](CO)COC1CCCC1 ; Cc1cccc(C)c1-c1cc(OC[C@H](N)COC2CCCC2)nc(NS(=O)(=O)c2cccc(C(=O)O)c2)n1]

To a solution of (2S)-2-amino-3-(cyclopentoxy)propan-1-ol (hydrochloride salt) (1.009 g, 4.8984 mmol) and 3-[[4-chloro-6-(2,6-dimethylphenyl)pyrimidin-2-yl]sulfamoyl]benzoic acid (2.012 g, 4.8149 mmol) in MeTHF (50 mL) and DMF (5 mL) at 10° C. was added portion-wise sodium tert-butoxide (2.845 g, 29.604 mmol). The reaction was stirred at 15° C. for 3 h. The mixture was cooled down to 5° C. with an ice-cold bath then slowly acidified with an aqueous solution of HCl 1 N (100 mL). The phases were separated, and the aqueous layer was extracted with Me-THF (2×50 mL). The combined organic layers were dried over anhydrous sodium sulfate, filtered, and concentrated under reduced pressure. Purification by reverse-phase chromatography (Column: C₁₈. Gradient: 5-30% acetonitrile in acidic water (0.1% w/w of HCl)) afforded 3-[[4-[(2R)-2-amino-3-(cyclopentoxy)propoxy]-6-(2,6-dimethylphenyl)pyrimidin-2-yl]sulfamoyl]benzoic acid (hydrochloride salt) (2.59 g, 93%) as a beige solid. ¹H NMR (400 MHz, DMSO-d6 with TFA) δ 8.44 (t, J=1.6 Hz, 1H), 8.24 (br. d, J=3.4 Hz, 3H), 8.17-8.12 (m, 2H), 7.69 (t, J=7.8 Hz, 1H), 7.25 (t, J=8.1 Hz, 1H), 7.12 (d, J=7.6 Hz, 2H), 6.31 (s, 1H), 4.36 (d, J=5.1 Hz, 2H), 3.98-3.91 (m, 1H), 3.74-3.65 (m, 1H), 3.64-3.58 (m, 1H), 3.57-3.51 (m, 1H), 2.00 (br. s, 6H), 1.73-1.53 (m, 6H), 1.52-1.41 (m, 2H), 2H missing, exchangeable protons. ESI-MS m/z calc. 540.2043, found 541.0 (M+1)⁺; Retention time: 1.4 minutes; LC method I.

### Step 5: 3-[[4-[(2R)-3-(Cyclopentoxy)-2-[(6-isopropyl-5-methyl-pyrrolo[2,3-b]pyrazin-3-yl)methylamino]propoxy]-6-(2,6-dimethylphenyl)pyrimidin-2-yl]sulfamoyl]benzoic acid

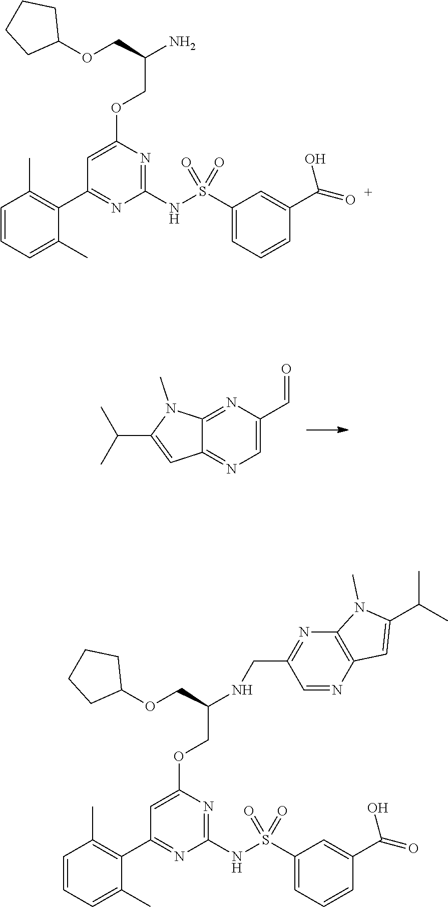 [SMILES: Cc1cccc(C)c1-c1cc(OC[C@H](N)COC2CCCC2)nc(NS(=O)(=O)c2cccc(C(=O)O)c2)n1 ; CC(C)c1cc2ncc(C=O)nc2n1C ; Cc1cccc(C)c1-c1cc(OC[C@@H](COC2CCCC2)NCc2cnc3cc(C(C)C)n(C)c3n2)nc(NS(=O)(=O)c2cccc(C(=O)O)c2)n1]

In a 4 mL vial, to a stirred mixture of 3-[[4-[(2R)-2-amino-3-(cyclopentoxy)propoxy]-6-(2,6-dimethylphenyl)pyrimidin-2-yl]sulfamoyl]benzoic acid; hydrochloride (45 mg, 0.07798 mmol) and 6-isopropyl-5-methyl-pyrrolo[2,3-b]pyrazine-3-carbaldehyde (15.9 mg, 0.07823 mmol) in anhydrous dichloromethane (300 μL) were added glacial acetic acid (10 μL, 0.1758 mmol) and DIPEA (40 μL, 0.2296 mmol), in that order, at 0° C. under nitrogen. After 2 min, sodium triacetoxyborohydride (55 mg, 0.2595 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish product as a light-brown solid. 3-[[4-[(2R)-3-(cyclopentoxy)-2-[(6-isopropyl-5-methyl-pyrrolo[2,3-b]pyrazin-3-yl)methylamino]propoxy]-6-(2,6-dimethylphenyl)pyrimidin-2-yl]sulfamoyl]benzoic acid (hydrochloride salt) (41 mg, 69%) ESI-MS m/z calc. 727.3152, found 728.9 (M+1)⁺; Retention time: 1.56 minutes. LC method A.

### Step 6: (11R)-11-(Cyclopentoxymethyl)-6-(2,6-dimethylphenyl)-12-[(6-isopropyl-5-methyl-pyrrolo[2,3-b]pyrazin-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-121

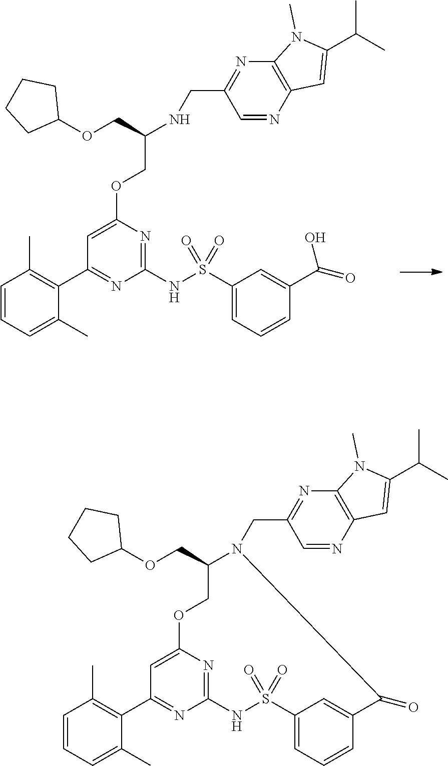 [SMILES: Cc1cccc(C)c1-c1cc(OC[C@@H](COC2CCCC2)NCc2cnc3cc(C(C)C)n(C)c3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3cc(C(C)C)n(C)c3n1)[C@H](COC1CCCC1)CO2]

To a stirred solution of 3-[[4-[(2R)-3-(cyclopentoxy)-2-[(6-isopropyl-5-methyl-pyrrolo[2,3-b]pyrazin-3-yl)methylamino]propoxy]-6-(2,6-dimethylphenyl)pyrimidin-2-yl]sulfamoyl]benzoic acid (hydrochloride salt) (41 mg, 0.05364 mmol) in anhydrous DMF (1.9 mL) was added 2-chloro-4,6-dimethoxy-1,3,5-triazine (15 mg, 0.08543 mmol) (CDMT), followed by addition of 4-methylmorpholine (40 μL, 0.3638 mmol) at 0-4° C. (ice-water bath) under nitrogen. The yellow reaction was allowed to stir at that temperature for 5 min, then allowed to stir at room temperature for 2 h. Purification of the reaction mixture by preparative reverse-phase HPLC (1-99% acetonitrile in water over 15 min, 5 mM HCl as modifier) furnished as a yellow solid. (11R)-11-(cyclopentoxymethyl)-6-(2,6-dimethylphenyl)-12-[(6-isopropyl-5-methyl-pyrrolo[2,3-b]pyrazin-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (24 mg, 62%) ¹H NMR (400 MHz, CDCl₃) δ 8.85 (t, J=1.8 Hz, 1H), 8.45 (s, 1H), 7.80 (dt, J=7.6, 1.4 Hz, 1H), 7.51 (t, J=7.8 Hz, 1H), 7.41 (d, J=8.0 Hz, 1H), 7.28 (t, J=7.6 Hz, 1H), 7.09 (d, J=7.6 Hz, 2H), 6.54 (s, 1H), 6.21 (s, 1H), 5.53-5.41 (m, 2H), 4.69 (d, J=15.9 Hz, 1H), 4.39-4.24 (m, 2H), 3.92 (s, 3H), 3.86-3.78 (m, 1H), 3.67-3.53 (m, 2H), 3.17 (hept, J=6.7 Hz, 1H), 2.08 (s, 6H), 1.73-1.61 (m, 5H), 1.61-1.47 (m, 3H), 1.40 (d, J=6.8 Hz, 3H), 1.38 (d, J=6.8 Hz, 3H). ESI-MS m/z calc. 709.3046, found 710.8 (M+1)⁺; Retention time: 1.98 minutes. LC method A.

### Example 68: Preparation of Compound I-123 and Compound I-154

### Step 1: Methyl (2S)-2-(tert-butoxycarbonylamino)-3-chloro-propanoate

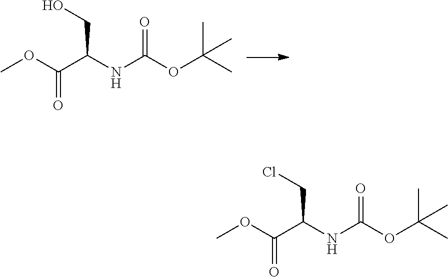

A solution of triphenylphosphine (6.58 g, 25.087 mmol) and hexachloroethane (5.94 g, 25.091 mmol) in DCM (85 mL) was added in one portion to a solution of methyl (2R)-2-(tert-butoxycarbonylamino)-3-hydroxy-propanoate (5 g, 22.807 mmol) in DCM (17 mL) under argon atmosphere. The reaction mixture was stirred at room temperature for 2 h and then the reaction was quenched with a saturated aqueous solution of sodium bicarbonate (15 mL). The product was extracted with dichloromethane (2×30 mL) and the combined organic layers was washed with brine (30 mL) and dried over anhydrous sodium sulphate. The solution was concentrated under reduced pressure and then triturated with diethyl ether (50 mL). After filtration and evaporation of the solvent, the crude product was purified by chromatography on a 80 g silica gel cartridge, eluting from 0% to 20% of ethyl acetate in heptanes to give methyl (2S)-2-(tert-butoxycarbonylamino)-3-chloro-propanoate (3.93 g, 72%) as a colorless oil. ¹H NMR (400 MHz, CDCl₃) δ 5.55-5.34 (m, 1H), 4.81-4.65 (m, 1H), 4.02-3.83 (m, 2H), 3.82 (s, 3H), 1.47 (s, 9H). ESI-MS m/z calc. 237.07678, found null (M+)⁺; 138.2 (M−99)⁺; Retention time: 1.7 minutes; LC method I.

### Step 2: tert-Butyl N-[(1S)-1-(chloromethyl)-2-hydroxy-ethyl]carbamate

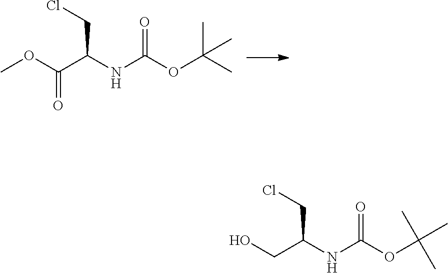

To a solution of methyl (2S)-2-(tert-butoxycarbonylamino)-3-chloro-propanoate (3.93 g, 16.535 mmol) in ethanol (47 mL) and THE (9 mL) at 0° C. was added lithium borohydride (441 mg, 18.220 mmol) portionwise. The reaction mixture was slowly warmed up to room temperature and further stirred overnight. The reaction was quenched with a saturated aqueous solution of ammonium chloride (5 ml). The volatiles were then evaporated, and the residue partitioned between water (60 ml) and EtOAc (60 ml). After separation, the aqueous phase was further extracted with EtOAc (60 ml). The combined organic layers were dried over sodium sulfate, filtered and concentrated over high vacuum to give tert-butyl N-[(1S)-1-(chloromethyl)-2-hydroxy-ethyl]carbamate (3.53 g, 94%) as a pale yellow oil. ¹H NMR (400 MHz, CDCl₃) δ 5.01 (br. s., 1H), 4.08-3.90 (m, 1H), 3.89-3.81 (m, 1H), 3.80-3.58 (m, 3H), 2.13 (br. s., 1H), 1.46 (s, 9H). ESI-MS m/z calc. 209.08188, found 110.2 (M−99)⁺; Retention time: 1.5 minutes; LC method I.

### Step 3: tert-Butyl N-[(1S)-1-(chloromethyl)-2-(2-trimethylsilylethoxymethoxy)ethyl]carbamate

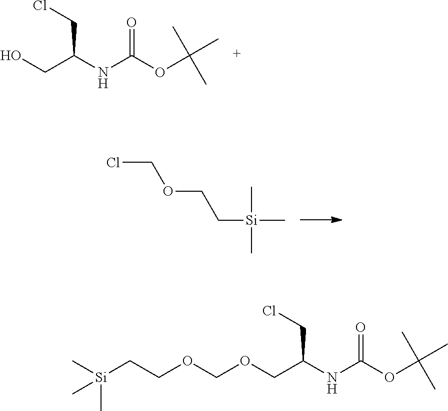

To a solution of tert-butyl N-[(1S)-1-(chloromethyl)-2-hydroxy-ethyl]carbamate (3.53 g, 16.836 mmol) in DCM (70 mL) at 0° C., was added diisopropylethylamine (4.6004 g, 6.2 mL, 35.595 mmol) followed by 2-(chloromethoxy)ethyl-trimethyl-silane (5.2752 g, 5.6 mL, 31.641 mmol) dropwise. The cooling bath was removed, and the reaction was stirred at rt overnight. The reaction mixture was concentrated under vacuum and then diluted with EtOAc (100 mL) then water (50 mL) was added, and the organic phase was separated. The product was extracted with EtOAc (100 mL) and the combine organic layers were washed with brine (50 mL) and dried over anhydrous sodium sulphate, filtered and concentrated under vacuum. The residue was purified by flash chromatography on a 80 g silica gel cartridge, eluting from 0% to 15% of ethyl acetate in heptanes to give tert-butyl N-[(1S)-1-(chloromethyl)-2-(2-trimethylsilylethoxy methoxy)ethyl]carbamate (4.175 g, 73%) ¹H NMR (400 MHz, CDCl₃) δ 5.11-4.96 (m, 1H), 4.69 (s, 2H), 4.02 (br. s., 1H), 3.81 (dd, J=10.0, 3.9 Hz, 1H), 3.76-3.69 (m, 1H), 3.67-3.53 (m, 4H), 1.46 (s, 9H), 1.02-0.92 (m, 2H), 0.04 (s, 9H). ESI-MS m/z calc. 339.16327, found 0.0 (M+1)⁺; Retention time: 2.11 minutes; LC method I.

### Step 4: tert-Butyl N-[(1R)-1-[(1-hydroxycyclobutyl)methyl]-2-(2-trimethylsilylethoxymethoxy)ethyl]carbamate

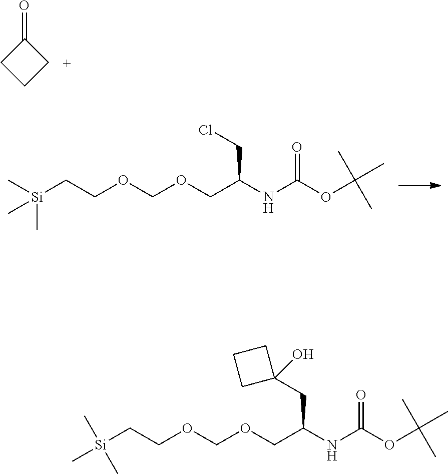

To a stirred solution of tert-butyl N-[(1S)-1-(chloromethyl)-2-(2-trimethylsilylethoxymethoxy)ethyl]carbamate (2.89 g, 8.5017 mmol) in dry THE (115 mL) under N₂ atmosphere, cooled at −78° C., was added dropwise n-BuLi in hexanes (13.9 mL of 1.6 M, 22.240 mmol). The stirring was continued for 15 min then lithium 2H-naphthalen-2-ide in THE (52 mL of 1 M, 52.000 mmol) was added dropwise over 5 min. The solution was stirred at −78° C. for one hour then a solution of cyclobutanone (1.5946 g, 1.7 mL, 22.751 mmol) in THE (15 mL) (on molecular sieves overnight) was added dropwise. The reaction mixture was stirred at −78° C. for 16 h then quenched with dropwise addition of a saturated aqueous solution of ammonium chloride (100 mL) and allowed to reach rt. The reaction was diluted with MTBE (150 mL) and a saturated aqueous solution of ammonium chloride (50 mL). The layers were separated, and the aqueous layer was extracted with MTBE (2×150 mL). The combined organic layers were dried over sodium sulfate, filtered and concentrated under reduced pressure. The residue was purified by chromatography on a 120 g silica gel cartridge, eluting from 0% to 20% of ethyl acetate in heptanes to give tert-butyl N-[(1R)-1-[(1-hydroxycyclobutyl)methyl]-2-(2-trimethylsilylethoxymethoxy)ethyl]carbamate (2.85 g, 89%) as a colorless oil. ESI-MS m/z calc. 375.2441, found 398.2 (M+23)⁺; null (M−)⁺; Retention time: 2.05 minutes; LC method I.

### Step 5: tert-Butyl N-[(1R)-1-[(1-fluorocyclobutyl)methyl]-2-(2-trimethylsilylethoxymethoxy)ethyl]carbamate

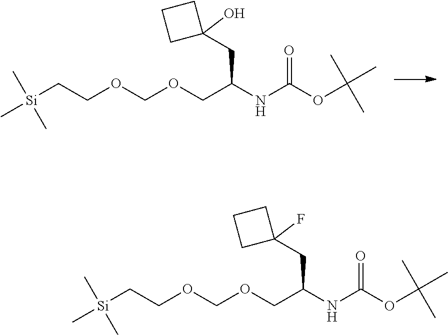

tert-Butyl N-[(1R)-1-[(1-hydroxycyclobutyl)methyl]-2-(2-trimethylsilylethoxy methoxy)ethyl]carbamate (1.2 g, 3.1951 mmol) was dissolved in DCM (30 mL). To this solution, was added pyridine (508.56 mg, 0.52 mL, 6.4293 mmol) followed by deoxofluor in THE (882.30 mg, 1.73 mL of 50% w/w, 1.9940 mmol). The resulting mixture was stirred at rt overnight. The reaction was not completed. Another addition of pyridine (254.28 mg, 0.26 mL, 3.2147 mmol) followed by deoxofluor in THE (351.90 mg, 0.69 mL of 50% w/w, 0.7953 mmol) was added to complete the reaction. The reaction was stirred one hour at rt. The reaction mixture was diluted with DCM (30 mL). The reaction mixture was washed with 10% citric acid (aq) (30 mL) and sat. sodium bicarbonate (aq.) (30 mL). The organic phase was dried over sodium sulfate, filtered, and evaporated. The residue was purified by chromatography on a 40 g silica gel cartridge, eluting from 0% to 30% of ethyl acetate in heptanes to give tert-butyl N-[(1R)-1-[(1-fluorocyclobutyl)methyl]-2-(2-trimethylsilylethoxymethoxy)ethyl]carbamate (266 mg, 22%) as a colorless oil. ¹H NMR (400 MHz, CDCl₃) δ 4.95 (br. s., 1H), 4.68 (s, 2H), 3.94 (br. s., 1H), 3.67-3.59 (m, 3H), 3.59-3.50 (m, 1H), 2.40-2.22 (m, 3H), 2.20-2.08 (m, 1H), 2.07-1.79 (m, 4H), 1.45 (s, 9H), 1.01-0.93 (m, 2H), 0.03 (s, 9H). 19F NMR (377 MHz, CDCl₃) δ −130.13-131.67 (m, 1F). ESI-MS m/z calc. 377.23978, found 400.2 (M+23)⁺; Retention time: 2.18 minutes; LC method I.

### Step 6: (2R)-2-Amino-3-(1-fluorocyclobutyl)propan-1-ol

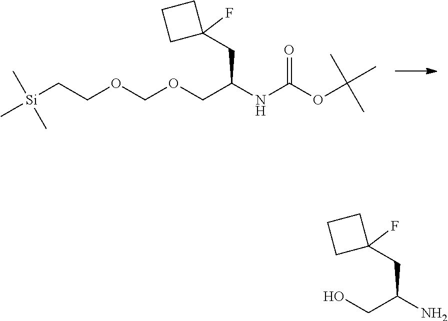 [SMILES: CC(C)(C)OC(=O)N[C@@H](COCOCC[Si](C)(C)C)CC1(F)CCC1 ; N[C@@H](CO)CC1(F)CCC1]

To a solution of tert-butyl N-[(1R)-1-[(1-fluorocyclobutyl)methyl]-2-(2-trimethylsilylethoxymethoxy)ethyl]carbamate (266 mg, 0.7045 mmol) dissolved in DCM (6 mL) was added TFA (1.4800 g, 1 mL, 12.980 mmol). The reaction was stirred at rt for 2 hours. The reaction was concentrated to dryness under vacuum to give (2R)-2-amino-3-(1-fluorocyclobutyl)propan-1-ol (trifluoroacetate salt) (184 mg, 100%) as a crude brown oil. ESI-MS m/z calc. 147.10594, found 148.2 (M+1)⁺; Retention time: 0.34 minutes; LC method I.

### Step 7: 3-[[4-[(2R)-2-Amino-3-(1-fluorocyclobutyl)propoxy]-6-(2,6-dimethylphenyl)pyrimidin-2-yl]sulfamoyl]benzoic acid

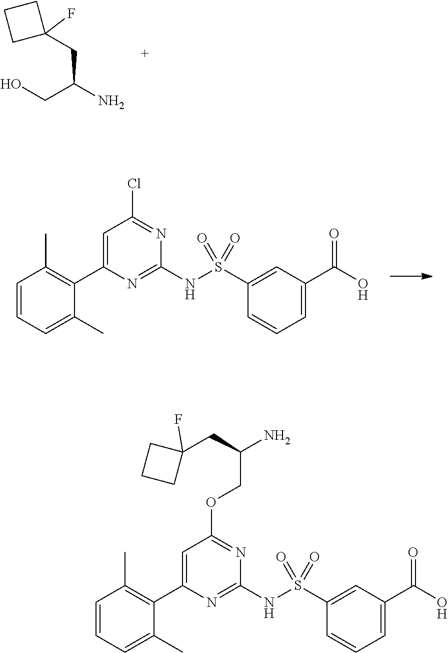 [SMILES: N[C@@H](CO)CC1(F)CCC1 ; Cc1cccc(C)c1-c1cc(Cl)nc(NS(=O)(=O)c2cccc(C(=O)O)c2)n1 ; Cc1cccc(C)c1-c1cc(OC[C@H](N)CC2(F)CCC2)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (88 mg, 0.2106 mmol) and (2R)-2-amino-3-(1-fluorocyclobutyl)propan-1-ol (trifluoroacetate salt) (45 mg, 0.1723 mmol) in 2-methyltetrahydrofuran (11.3 mL) and DMF (1.1 mL) at 0° C. was added sodium tert-butoxide (100 mg, 1.0405 mmol) in one portion and the mixture was stirred at room temperature for 0.5 hours. A second amount of sodium tert-butoxide (50 mg, 0.5203 mmol) was added at 0° C. The reaction was stirred at room temperature for one hour. The mixture was acidified with iN hydrochloric acid (5 mL) at 0° C. and the phases were separated. The aqueous layer was extracted with EtOAc (3×10 mL), the combined organic phases were dried over sodium sulfate, filtered, and concentrated to dryness. The residue was purified by reverse phase chromatography on a 15.5 g C₁₈ Gold cartridge using MeCN in acidic water (contains 0.1% w/w HCl) 5% for 1 CV then to 100% over 25CV. The product was dissolved in minimum of acetonitrile and water and freeze-dried overnight to provide 3-[[4-[(2R)-2-amino-3-(1-fluorocyclobutyl)propoxy]-6-(2,6-dimethylphenyl)pyrimidin-2-yl]sulfamoyl]benzoic acid (hydrochloride salt) (22 mg, 15%) as a white solid. ESI-MS m/z calc. 528.18427, found 529.1 (M+1)⁺; Retention time: 2.36 minutes; LC method J.

### Step 8: 3-[[4-[(2R)-2-[(6-cyclopropylfuro[2,3-b]pyrazin-2-yl)methylamino]-3-(1-fluorocyclobutyl)propoxy]-6-(2,6-dimethylphenyl)pyrimidin-2-yl]sulfamoyl]benzoic acid, and 3-[[4-[(2R)-3-cyclobutylidene-2-[(6-cyclopropylfuro[2,3-b]pyrazin-2-yl)methylamino]propoxy]-6-(2,6-dimethylphenyl)pyrimidin-2-yl]sulfamoyl]benzoic acid

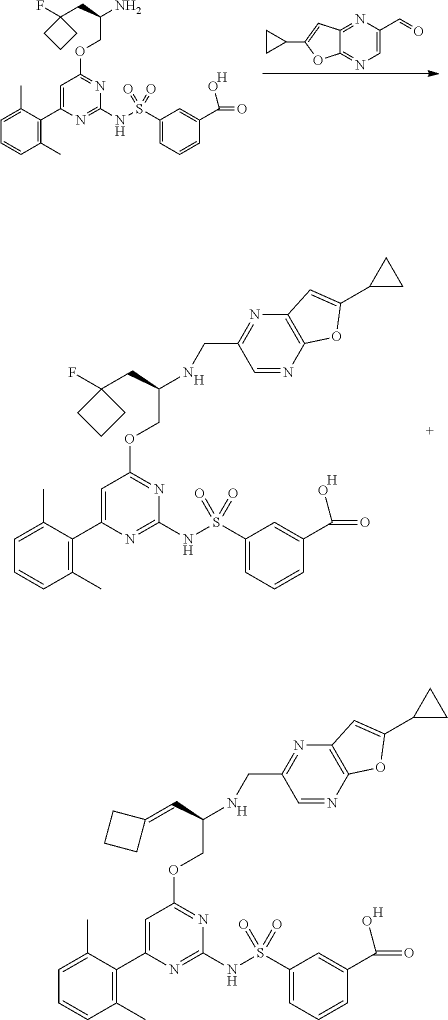

In a 4 mL vial, to a stirred mixture of 3-[[4-[(2R)-2-amino-3-(1-fluorocyclobutyl)propoxy]-6-(2,6-dimethylphenyl)pyrimidin-2-yl]sulfamoyl]benzoic acid; hydrochloride (40 mg, 0.07079 mmol) and 6-cyclopropylfuro[2,3-b]pyrazine-2-carbaldehyde (13.4 mg, 0.07121 mmol) in anhydrous dichloromethane (300 μL) were added glacial acetic acid (10 μL, 0.1758 mmol) and DIPEA (40 μL, 0.2296 mmol), in that order, at 0° C. under nitrogen. After 2 min, sodium triacetoxyborohydride (55 mg, 0.2595 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish two fractions, both as a white solid. 3-[[4-[(2R)-2-[(6-cyclopropylfuro[2,3-b]pyrazin-2-yl)methylamino]-3-(1-fluorocyclobutyl)propoxy]-6-(2,6-dimethylphenyl)pyrimidin-2-yl]sulfamoyl]benzoic acid (hydrochloride salt) (20 mg, 38%) ESI-MS m/z calc. 700.2479, found 701.8 (M+1)⁺; Retention time: 1.53 minutes. and 3-[[4-[(2R)-3-cyclobutylidene-2-[(6-cyclopropylfuro[2,3-b]pyrazin-2-yl)methylamino]propoxy]-6-(2,6-dimethylphenyl)pyrimidin-2-yl]sulfamoyl]benzoic acid (hydrochloride salt) (9 mg, 18%) ESI-MS m/z calc. 680.2417, found 681.6 (M+1)⁺; Retention time: 1.44 minutes. LC method A.

### Step 9: (11R)-12-[(6-cyclopropylfuro[2,3-b]pyrazin-2-yl)methyl]-6-(2,6-dimethylphenyl)-11-[(1-fluorocyclobutyl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one, Compound I-123, and (11R)-11-(cyclobutylidenemethyl)-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,4,16-hexaen-13-one, Compound I-154

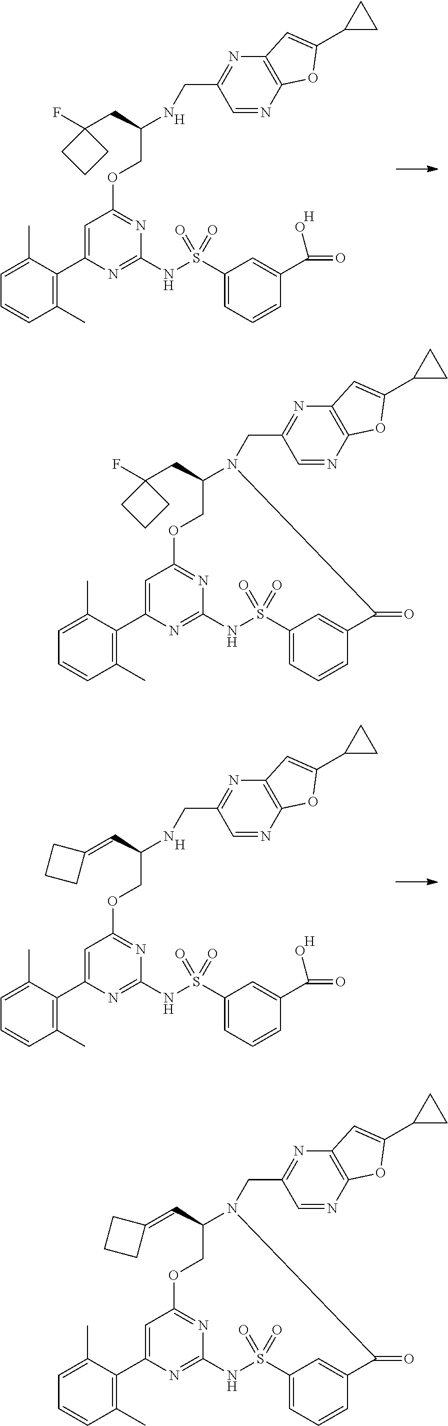

Two separate reactions were carried out. Reaction 1: To a stirred solution of 3-[[4-[(2R)-2-[(6-cyclopropylfuro[2,3-b]pyrazin-2-yl)methylamino]-3-(1-fluorocyclobutyl)propoxy]-6-(2,6-dimethylphenyl)pyrimidin-2-yl]sulfamoyl]benzoic acid (hydrochloride salt) (20 mg, 0.02713 mmol) in anhydrous DMF (1 mL) was added 2-chloro-4,6-dimethoxy-1,3,5-triazine (8 mg, 0.04557 mmol) (CDMT), followed by addition of 4-methylmorpholine (20 μL, 0.1819 mmol) at 0-4° C. (ice-water bath) under nitrogen. The yellow reaction was allowed to stir at that temperature for 5 min, then allowed to stir at room temperature for 2 h. Reaction 2: To a stirred solution of 3-[[4-[(2R)-3-cyclobutylidene-2-[(6-cyclopropylfuro[2,3-b]pyrazin-2-yl)methylamino]propoxy]-6-(2,6-dimethylphenyl)pyrimidin-2-yl]sulfamoyl]benzoic acid (hydrochloride salt) (9 mg, 0.01255 mmol) in anhydrous DMF (0.5 mL) was added 2-chloro-4,6-dimethoxy-1,3,5-triazine (4 mg, 0.02278 mmol) (CDMT), followed by addition of 4-methylmorpholine (10 μL, 0.09096 mmol) at 0-4° C. (ice-water bath) under nitrogen. The yellow reaction was allowed to stir at that temperature for 5 min, then allowed to stir at room temperature for 2 h. The two reactions were combined, as both had the other component in significant amounts, and purified by preparative reverse-phase HPLC (1-99% acetonitrile in water over 15 min, 5 mM HCl as modifier) to furnish two products as white solids. First compound eluted (11R)-12-[(6-cyclopropylfuro[2,3-b]pyrazin-2-yl)methyl]-6-(2,6-dimethylphenyl)-11-[(1-fluorocyclobutyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (10 mg, 53%) ¹H NMR (400 MHz, CDCl₃) δ 9.00 (s, 1H), 8.65 (t, J=1.8 Hz, 1H), 8.35 (s, 1H), 7.90 (d, J=7.9 Hz, 1H), 7.78 (d, J=7.6 Hz, 1H), 7.58 (t, J=7.8 Hz, 1H), 7.22 (t, J=7.6 Hz, 1H), 7.06 (d, J=7.6 Hz, 2H), 6.63 (s, 1H), 6.24 (s, 1H), 5.38 (dd, J=10.9, 3.9 Hz, 1H), 5.32 (d, J=14.9 Hz, 1H), 4.46-4.35 (m, 1H), 4.31 (t, J=11.3 Hz, 1H), 4.25 (d, J=14.9 Hz, 1H), 2.32-2.16 (m, 2H), 2.16-2.04 (m, 2H), 2.02 (s, 6H), 2.00-1.89 (m, 2H), 1.84-1.71 (m, 1H), 1.50-1.40 (m, 1H), 1.39-1.31 (m, 1H), 1.20-1.10 (m, 4H). ESI-MS m/z calc. 682.23737, found 683.7 (M+1)⁺; Retention time: 2.03 minutes. and the second compound eluted (11R)-11-(cyclobutylidenemethyl)-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (2.5 mg, 14%) ¹H NMR (400 MHz, CDCl₃) δ 8.65 (t, J=1.8 Hz, 1H), 8.32 (s, 1H), 8.08 (d, J=7.9 Hz, 1H), 7.85 (dt, J=7.7, 1.4 Hz, 1H), 7.65 (t, J=7.8 Hz, 1H), 7.21 (t, J=7.6 Hz, 1H), 7.05 (d, J=7.6 Hz, 2H), 6.61 (s, 1H), 6.21 (s, 1H), 5.38-5.28 (m, 2H), 5.25 (d, J=15.1 Hz, 1H), 4.71-4.60 (m, 1H), 4.46-4.35 (m, 2H), 2.72-2.53 (m, 2H), 2.35-2.21 (m, 1H), 2.18-2.09 (m, 1H), 2.01 (s, 6H), 1.96-1.82 (m, 2H), 1.82-1.70 (m, 1H), 1.22-1.10 (m, 4H). ESI-MS m/z calc. 662.23114, found 663.6 (M+1)⁺; Retention time: 1.96 minutes. LC method A.

### Example 69: Preparation of Compound 126

### Step 1: 2-Chloro-6-isopropyl-7-(trifluoromethyl)furo[2,3-b]pyrazine

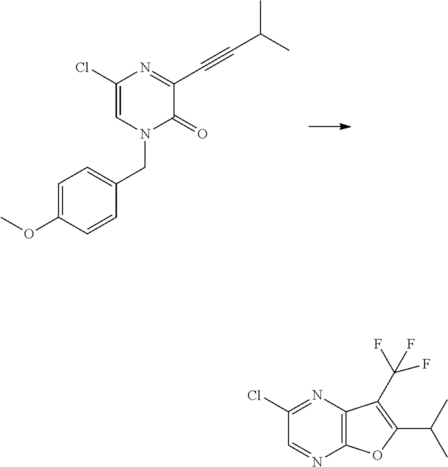 [SMILES: COc1ccc(Cn2cc(Cl)nc(C#CC(C)C)c2=O)cc1 ; CC(C)c1oc2ncc(Cl)nc2c1C(F)(F)F]

To an oven dried microwave vial was added 5-chloro-1-[(4-methoxyphenyl)methyl]-3-(3-methylbut-1-ynyl)pyrazin-2-one (5 g, 15.784 mmol), Umemoto's reagent (9.57 g, 23.668 mmol), CuBr (2.28 g, 15.894 mmol) under nitrogen. Then anhydrous DMA (13.3 mL) was added, and the mixture was heated at 50° C.30 minutes then the reaction was stirred for 12 h at room temperature. To the reaction was added 1 M HCl (50 mL) and extracted with diethyl ether (3×100 mL), washed with brine (2×100 mL), dried over MgSO₄, filtered and concentrated in vacuo. The residue was purified by flash chromatography (loaded onto a 330 g silica cartridge with toluene, and eluted with 0-1% diethyl ether in hexanes over a 20 min gradient, then held at 1% diethyl ether in hexanes for 40 minutes) to provide 2-chloro-6-isopropyl-7-(trifluoromethyl)furo[2,3-b]pyrazine (2.145 g, 41%) as a colorless solid ¹H NMR (500 MHz, Chloroform-d) δ 8.28 (s, 1H), 3.62-3.51 (m, 1H), 1.44 (d, J=6.8 Hz, 6H). ESI-MS m/z calc. 264.0277, found 265.0 (M+1)⁺; Retention time: 5.61 minutes; LC method D.

### Step 2: 6-Isopropyl-7-(trifluoromethyl)-2-vinyl-furo[2,3-b]pyrazine

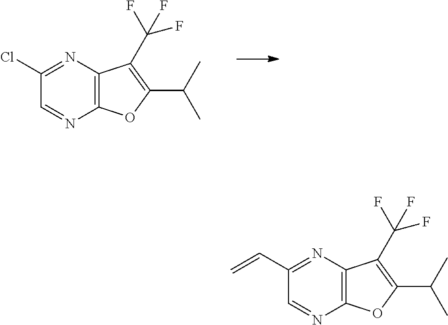 [SMILES: CC(C)c1oc2ncc(Cl)nc2c1C(F)(F)F ; C=Cc1cnc2oc(C(C)C)c(C(F)(F)F)c2n1]

Under an atmosphere of argon, 2-chloro-6-isopropyl-7-(trifluoromethyl)furo[2,3-b]pyrazine (1.95 g, 6.2634 mmol), potassium vinyltrifluoroborate (2.47 g, 18.440 mmol) and Pd(dppf)Cl₂ (419.0 mg, 0.5131 mmol) were added to a vial. To the mixture was added degassed EtOH (58 mL) and TEA (3.3614 g, 4.63 mL, 33.219 mmol). The mixture was further degassed for 5 minutes, sealed under argon, and heated in a 90° C. oil bath for 30 minutes. The reaction was cooled to room temperature and concentrated in vacuo. The residue was purified by flash chromatography (loaded onto a 220 g silica cartridge with toluene, and eluted with 0-3.5% diethyl ether in hexanes over a 50 min gradient) to provide 6-isopropyl-7-(trifluoromethyl)-2-vinyl-furo[2,3-b]pyrazine (1 g, 62%) as a white solid. ESI-MS m/z calc. 256.08234, found 257.4 (M+1)⁺; Retention time: 5.76 minutes; LC method D.

### Step 3: 6-Isopropyl-7-(trifluoromethyl)furo[2,3-b]pyrazine-2-carbaldehyde

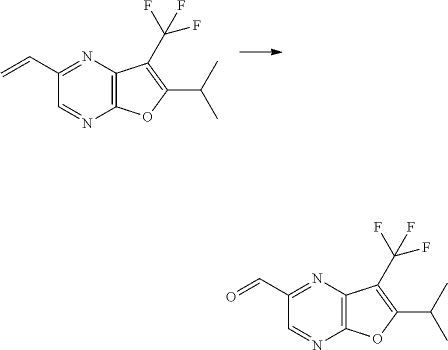 [SMILES: C=Cc1cnc2oc(C(C)C)c(C(F)(F)F)c2n1 ; CC(C)c1oc2ncc(C=O)nc2c1C(F)(F)F]

To a solution of 6-isopropyl-7-(trifluoromethyl)-2-vinyl-furo[2,3-b]pyrazine (120 mg, 0.4683 mmol) in dioxane (1.20 mL) H₂O (0.408 mL) was added pyridine (74.328 mg, 0.076 mL, 0.9397 mmol), OsO₄ in tBuOH (0.240 mL of 2.5% w/v, 0.0236 mmol) then NaIO₄ (400 mg, 1.8701 mmol) at room temperature. The reaction was stirred at room temperature 2 h. Then OsO₄ in tBuOH (1.2 mL of 2.5% w/v, 0.1180 mmol) and pyridine (146.70 mg, 0.150 mL, 1.8546 mmol) were added. The reaction was stirred at room temperature for 4 h. The reaction was diluted with DCM (20 mL) washed with saturated aqueous sodium bicarbonate (20 mL), extracted with DCM (3×15 mL), washed with brine (40 mL) dried over sodium sulfate filtered and concentrated in vacuo. The residue was purified by flash chromatography (loaded onto a 12 g silica cartridge with toluene, and eluted with 0-10% EtOAc in hexanes over a 30 min gradient) to provide 6-isopropyl-7-(trifluoromethyl)furo[2,3-b]pyrazine-2-carbaldehyde (64.6 mg, 53%) as a colorless oil ¹H NMR (500 MHz, Chloroform-d) δ 10.24 (s, 1H), 8.97 (s, 1H), 3.63 (hept, J=7.0 Hz, 1H), 1.47 (d, J=7.0 Hz, 6H). ¹⁹F NMR (470 MHz, Chloroform-d) 6-56.92. ESI-MS m/z calc. 258.0616, found 259.3 (M+1)⁺; Retention time: 2.45 minutes; LC method H.

### Step 4: 3-[[4-(2,6-Dimethylphenyl)-6-[(2R)-2-[[6-isopropyl-7-(trifluoromethyl)furo[2,3-b]pyrazin-2-yl]methylamino]-3-(1-methylcyclopropyl)propoxy]pyrimidin-2-yl]sulfamoyl]benzoic acid

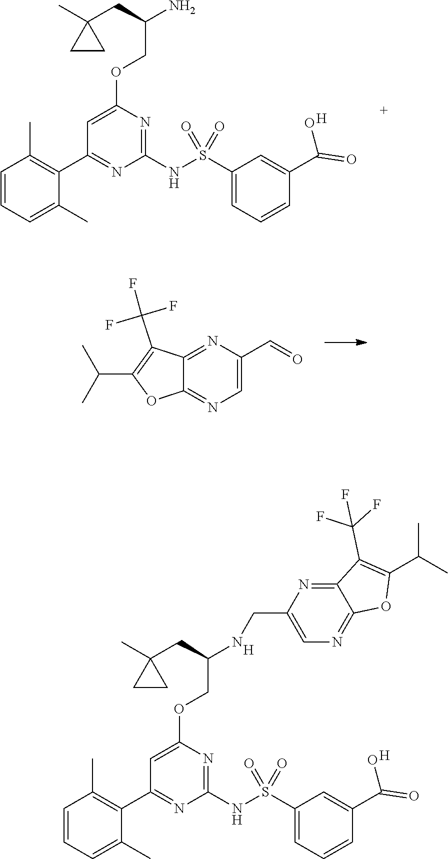

A 4 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (90 mg, 0.1645 mmol), 6-isopropyl-7-(trifluoromethyl)furo[2,3-b]pyrazine-2-carbaldehyde (43.0 mg, 0.1665 mmol), anhydrous DCM (0.75 mL), and acetic acid (13.2 μL, 0.2321 mmol). The mixture was cooled down in an ice bath. DIEA (60.2 μL, 0.3456 mmol) was added, followed by sodium triacetoxyborohydride (210.7 mg, 0.9941 mmol) and the reaction was vigorously stirred at 0° C. for 2 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-(2,6-dimethylphenyl)-6-[(2R)-2-[[6-isopropyl-7-(trifluoromethyl)furo[2,3-b]pyrazin-2-yl]methylamino]-3-(1-methylcyclopropyl)propoxy]pyrimidin-2-yl]sulfamoyl]benzoic acid (hydrochloride salt) (99.1 mg, 73%) as a white solid. ESI-MS m/z calc. 752.2604, found 753.6 (M+1)⁺; Retention time: 1.63 minutes; LC method A.

### Step 5: (11R)-6-(2,6-Dimethylphenyl)-12-[[6-isopropyl-7-(trifluoromethyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one (Compound I-126

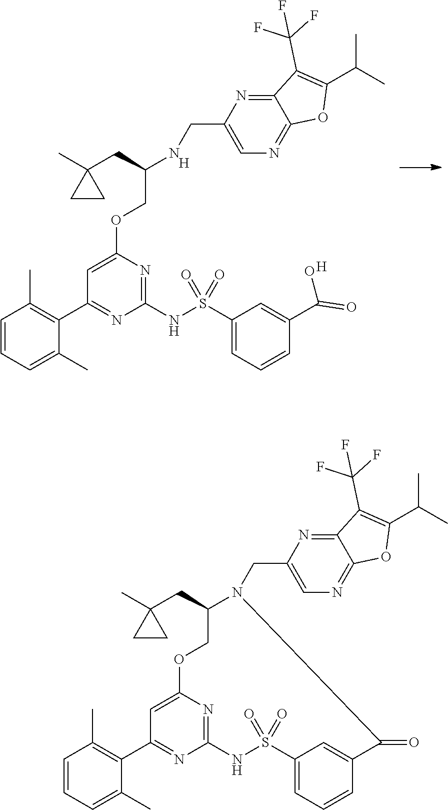

3-[[4-(2,6-Dimethylphenyl)-6-[(2R)-2-[[6-isopropyl-7-(trifluoromethyl)furo[2,3-b]pyrazin-2-yl]methylamino]-3-(1-methylcyclopropyl)propoxy]pyrimidin-2-yl]sulfamoyl]benzoic acid (hydrochloride salt) (99.1 mg, 0.1205 mmol) was combined with CDMT (21.9 mg, 0.1247 mmol) in DMF (10 mL) and cooled to 0° C. N-methylmorpholine (60.3 μL, 0.5485 mmol) was added by syringe and the reaction was stirred at 0° C. for 30 minutes. The ice bath was then removed and stirring was continued for an additional 16 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×130 mL with brine, dried over sodium sulfate, filtered, and concentrated. The crude product was diluted with DMSO and MeOH, filtered, and purified by reverse phase HPLC (1-99% acetonitrile/5 mM aqueous HCl over 25 min) to give (11R)-6-(2,6-dimethylphenyl)-12-[[6-isopropyl-7-(trifluoromethyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (32.5 mg, 36%) as a white solid. ¹H NMR (400 MHz, CDCl₃) δ 9.17-8.75 (m, OH), 8.63-8.58 (m, 2H), 7.99 (dt, J=7.9, 1.5 Hz, 1H), 7.84 (dt, J=7.6, 1.4 Hz, 1H), 7.59 (t, J=7.8 Hz, 1H), 7.22 (t, J=7.6 Hz, 1H), 7.06 (d, J=7.6 Hz, 2H), 6.25 (s, 1H), 5.38-5.30 (m, 1H), 5.26 (d, J=14.5 Hz, 1H), 4.50-4.40 (m, 2H), 4.20 (d, J=14.5 Hz, 1H), 3.56 (h, J=7.0 Hz, 1H), 2.02 (s, 6H), 1.84 (d, J=15.1 Hz, 1H), 1.47-1.42 (m, 7H), 0.44 (s, 3H), 0.36-0.29 (m, 1H), 0.27-0.19 (m, 1H), 0.15-0.08 (m, 1H), 0.03-0.00 (m, 1H). ¹⁹F NMR (376 MHz, CDCl₃) δ −56.68. ESI-MS m/z calc. 734.2498, found 735.1 (M+1)⁺; Retention time: 2.08 minutes; LC method A.

### Example 70: Preparation of Compound I-128

### Step 1: 6-Chloro-2-isopropyl-pyrazolo[3,4-b]pyrazine

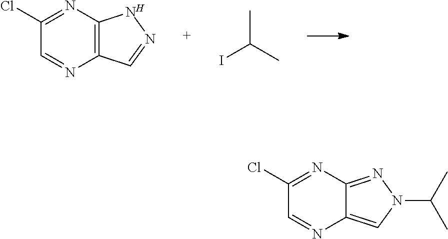

To a solution of 6-chloro-1H-pyrazolo[3,4-b]pyrazine (5 g, 31.380 mmol) in dry THE (50 mL) at 0° C. was added NaHMDS (1 M in THF) (50 mL of 1 M, 50.000 mmol) followed by 2-iodopropane (17.030 g, 10 mL, 100.18 mmol). The mixture was stirred at 0° C. for 5 minutes and then at 70° C. for 18 hours. The mixture was then cooled down to room temperature and the mixture was filtered on a pad of Celite. The pad of Celite was washed with EtOAc (250 mL) and the mixture was concentrated in vacuo. Water (250 mL) and EtOAc (250 mL) were added, and the organic layer was washed with water (2×250 mL) and brine (1×250 mL), dried over sodium sulfate, filtered and concentrated in vacuo. The crude mixture was purified by reverse phase chromatography on a 100 g C₁₈ GOLD cartridge, eluting with a gradient of 5 to 100% of MeCN in acidic water (0.1% v/v of formic acid in water). The fractions containing the desired product were combined and the organic solvent was evaporated. EtOAc (100 mL) was then added, and the mixture was extracted with EtOAc (3×100 mL). The combined organic layers were washed with brine (1×50 mL), dried over sodium sulfate, filtered and concentrated in vacuo to afford 6-chloro-2-isopropyl-pyrazolo[3,4-b]pyrazine (1.15 g, 19%) as a yellow solid. ¹H NMR (400 MHz, CDCl₃) δ 8.46 (s, 1H), 8.27 (s, 1H), 4.84 (spt, J=6.7 Hz, 1H), 1.69 (d, J=6.6 Hz, 6H). ESI-MS m/z calc. 196.05157, found 197.2 (M+1)⁺; Retention time: 1.54 minutes; LC method I.

### Step 2: 2-Isopropyl-6-vinyl-pyrazolo[3,4-b]pyrazine

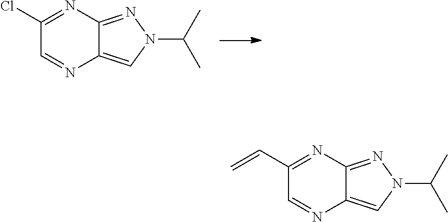

To a solution of 6-chloro-2-isopropyl-pyrazolo[3,4-b]pyrazine (1.15 g, 5.8425 mmol) in toluene (10 mL) was added tributyl(vinyl)stannane (3.7 g, 11.668 mmol) followed by lithium chloride (745 mg, 17.573 mmol) and copper iodide (225 mg, 1.1814 mmol). Nitrogen was bubbled into the mixture for 15 minutes and then Pd(PPh₃)₄ (675 mg, 0.5841 mmol) was added. Nitrogen was bubbled into the mixture for 10 minutes. The mixture was stirred at 80° C. for 4 hours and was then cooled down to room temperature. EtOAc (10 mL) was added, and the mixture was filtered on a pad of Celite. The pad was rinsed with EtOAc (100 mL) and the mixture was concentrated in vacuo. The crude mixture was purified by reverse phase chromatography on a 275 g C₁₈ GOLD cartridge, eluting with a gradient of 5 to 100% of MeCN in acidic water (0.1% v/v of formic acid in water). The fractions containing the desired product were combined and the organic solvent was evaporated. EtOAc (100 mL) was then added, and the mixture was extracted with EtOAc (3×100 mL). The combined organic layers were washed with brine (1×250 mL), dried over sodium sulfate, filtered and concentrated in vacuo to afford 2-isopropyl-6-vinyl-pyrazolo[3,4-b]pyrazine (970 mg, 86%) as a black oil. ¹H NMR (400 MHz, CDCl₃) δ 8.66 (s, 1H), 8.22 (s, 1H), 6.98 (dd, J=17.6, 11.0 Hz, 1H), 6.52 (d, J=17.6 Hz, 1H), 5.73 (d, J=11.0 Hz, 1H), 4.84 (spt, J=6.6 Hz, 1H), 1.71 (d, J=6.8 Hz, 6H). ESI-MS m/z calc. 188.1062, found 189.2 (M+1)⁺; Retention time: 1.45 minutes; LC method I.

### Step 3: 2-Isopropylpyrazolo[3,4-b]pyrazine-6-carbaldehyde

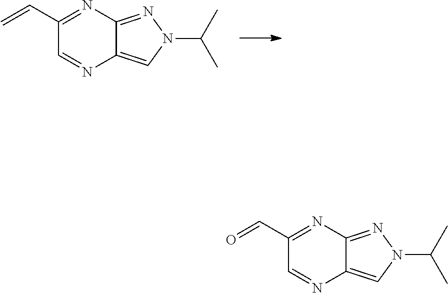

To a stirred solution of 2-isopropyl-6-vinyl-pyrazolo[3,4-b]pyrazine (950 mg, 4.9411 mmol) in a mixture of THF (20 mL) and water (10 mL) at 0° C. was added NMO (1.16 g, 9.9022 mmol) followed by OsO₄ (in tBuOH solution) (2.5 g, 2.5% w/w, 0.2458 mmol). The mixture was stirred at 0° C. for 5 minutes and then sodium periodate (4.25 g, 19.870 mmol) was added. The mixture was stirred at 0° C. for 15 minutes and then at room temperature for 2 hours. Water (100 mL) and EtOAc (100 mL) were added, and the mixture was extracted with EtOAc (3×100 mL). The combined organic layers were washed with brine (250 mL), dried over sodium sulfate, filtered and concentrated in vacuo. The crude mixture was purified by flash-chromatography on a silica gel cartridge (40 g Gold), eluting with a gradient of 0 to 100% of EtOAc in heptane to afford after evaporation 2-isopropylpyrazolo[3,4-b]pyrazine-6-carbaldehyde (500 mg, 53%) as a yellow solid. ¹H NMR (400 MHz, CDCl₃) δ 10.21 (s, 1H), 9.18 (s, 1H), 8.37 (s, 1H), 4.95 (spt, J=6.7 Hz, 1H), 1.76 (d, J=6.8 Hz, 6H). ESI-MS m/z calc. 190.08546, found 191.1 (M+1)⁺; Retention time: 2.08 minutes; LC method K.

### Step 4: 3-[[4-(2,6-Dimethylphenyl)-6-[(2R)-2-[(2-isopropylpyrazolo[3,4-b]pyrazin-6-yl)methylamino]-3-(1-methylcyclopropyl)propoxy]pyrimidin-2-yl]sulfamoyl]benzoic acid

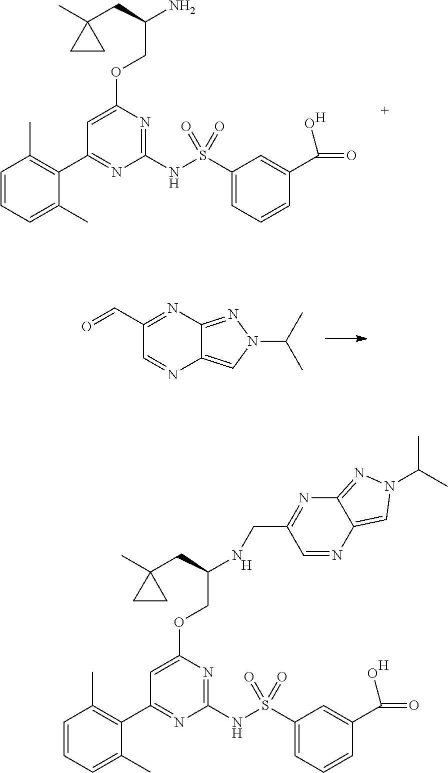

A 4 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (89.5 mg, 0.1636 mmol), 2-isopropylpyrazolo[3,4-b]pyrazine-6-carbaldehyde (31.5 mg, 0.1656 mmol), anhydrous DCM (0.75 mL), and acetic acid (13.9 μL, 0.2444 mmol). The mixture was cooled down in an ice bath. DIEA (59.9 μL, 0.3439 mmol) was added, followed by sodium triacetoxyborohydride (210.5 mg, 0.9932 mmol), and the reaction was vigorously stirred at 0° C. for 3 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-(2,6-dimethylphenyl)-6-[(2R)-2-[(2-isopropylpyrazolo[3,4-b]pyrazin-6-yl)methylamino]-3-(1-methylcyclopropyl)propoxy]pyrimidin-2-yl]sulfamoyl]benzoic acid (hydrochloride salt) (83.9 mg, 70%) as a red-white solid. ESI-MS m/z calc. 684.28424, found 685.9 (M+1)⁺; Retention time: 1.21 minutes. LC method A.

### Step 5: (11R)-6-(2,6-Dimethylphenyl)-12-[(2-isopropylpyrazolo[3,4-b]pyrazin-6-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-128-

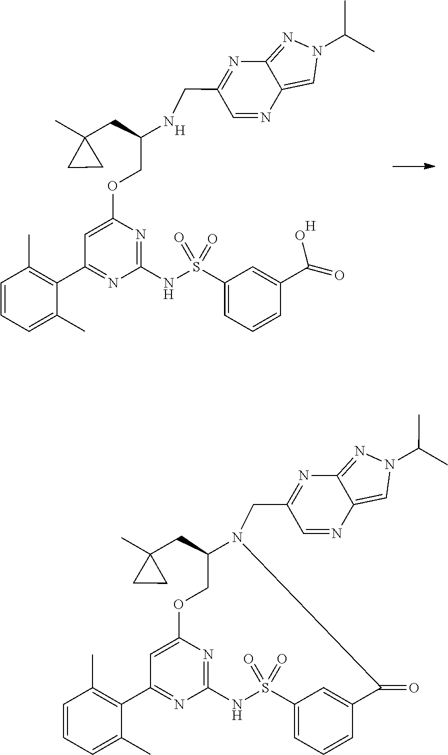

3-[[4-(2,6-Dimethylphenyl)-6-[(2R)-2-[(2-isopropylpyrazolo[3,4-b]pyrazin-6-yl)methylamino]-3-(1-methylcyclopropyl)propoxy]pyrimidin-2-yl]sulfamoyl]benzoic acid (hydrochloride salt) (83.9 mg, 0.1140 mmol) was combined with CDMT (21.9 mg, 0.1247 mmol) in DMF (9.5 mL) and cooled to 0° C. N-methylmorpholine (62.7 μL, 0.5703 mmol) was added by syringe and the reaction was stirred at 0° C. for 30 minutes. The ice bath was then removed and stirring was continued for an additional 22 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was purified by reverse phase HPLC (1-99% acetonitrile/5 mM aqueous HCl over 25 min) to give (11R)-6-(2,6-dimethylphenyl)-12-[(2-isopropylpyrazolo[3,4-b]pyrazin-6-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (28.9 mg, 38%) as a white solid. ¹H NMR (400 MHz, CDCl₃) δ 8.79 (s, 1H), 8.62-8.58 (m, 1H), 8.33 (s, 1H), 8.13 (dt, J=7.9, 1.5 Hz, 1H), 7.86 (dt, J=7.7, 1.4 Hz, 1H), 7.64 (t, J=7.8 Hz, 1H), 7.21 (t, J=7.6 Hz, 1H), 7.04 (d, J=7.6 Hz, 2H), 6.20 (s, 1H), 5.35 (d, J=14.4 Hz, 1H), 5.32-5.25 (m, 1H), 4.86 (hept, J=6.6 Hz, 1H), 4.54-4.39 (m, 2H), 4.16 (d, J=14.4 Hz, 1H), 1.99 (s, 6H), 1.84-1.77 (m, 1H), 1.71 (d, 6H), 1.59-1.49 (m, 1H), 0.48 (s, 3H), 0.37-0.30 (m, 1H), 0.29-0.20 (m, 1H), 0.17-0.10 (m, 1H), 0.05-0.01 (m, 1H). ESI-MS m/z calc. 666.2737, found 667.1 (M+1)⁺; Retention time: 1.67 minutes. LC method A.

### Example 71: Preparation of Compound III-20

### Step 1: tert-Butyl N-(5-iodothiazol-4-yl)carbamate

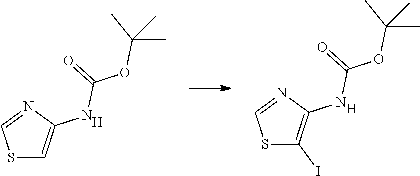

To a flame-dried flask (1000 mL) were added tert-butyl N-thiazol-4-ylcarbamate (6.5 g, 31.809 mmol), NIS (8.4 g, 36.589 mmol), and DCE (319 mL). The solution was stirred under N₂ at room temperature for 17 h. Then, the solution was washed with H₂O (2×50 mL) and saturated aqueous Na₂S₂O₃ solution (100 mL). The organic layer was dried with Sodium sulfate, and evaporated under reduced pressure to afford a crude product which was purified by flash chromatography on silica gel (column: 300 gram HP Gold; gradient: 5% to 30% ethyl acetate in heptane, 8 CV) to afford tert-butyl N-(5-iodothiazol-4-yl)carbamate (7.4553 g, 72%) as white crystals. ¹H NMR (400 MHz, CDCl₃) δ 8.88 (s, 1H), 6.63-6.44 (m, 1H), 1.54 (s, 9H). ESI-MS m/z calc. 325.9586, found null (M+)⁺; 271.0 (M−55)⁺; Retention time: 1.64 minutes; LC method I.

### Step 2: tert-Butyl N-[5-(3-methylbut-1-ynyl)thiazol-4-yl]carbamate

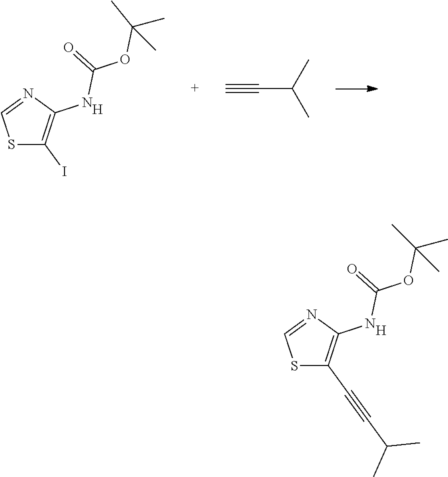

To a flame-dried flask (250 mL) was added THF (66 mL). Then, N₂ was bubbled into the solution for 15 minutes. After that, CuI (367 mg, 1.9270 mmol), Pd(dppf)Cl₂ (550 mg, 0.6735 mmol), cesium carbonate (11 g, 33.761 mmol) and tert-butyl N-(5-iodothiazol-4-yl)carbamate (6.63 g, 20.328 mmol) were added under N₂ atmosphere. The mixture was stirred at 50° C. for 15 minutes. Then, 3-methylbut-1-yne (1.3847 g, 2.7 mL, 20.328 mmol) in THE (3.5 mL) was added over 40 minutes. The reaction was stirred at 50° C. for 7 h. Then, the mixture was cooled down to room temperature and filtered through Celite pad and washed with ethyl acetate (100 mL), The filtrate was evaporated under reduced pressure to afford a crude product which was purified by chromatography on silica gel (column: 40 g HP Gold; gradient: 5 to 30% ethyl acetate in heptane; 12 CV) to afford tert-butyl N-[5-(3-methylbut-1-ynyl)thiazol-4-yl]carbamate (4.5 g, 82%) as a white powder. ¹H NMR (400 MHz, CDCl₃) δ 8.51 (s, 1H), 6.77 (br. s, 1H), 2.90-2.79 (m, 1H), 1.54 (s, 9H), 1.28 (d, J=6.8 Hz, 6H). ESI-MS m/z calc. 266.1089, found 211.2 (M−55)⁺; Retention time: 1.83 minutes; LC method I.

### Step 3: tert-Butyl 5-isopropylpyrrolo[2,3-d]thiazole-4-carboxylate

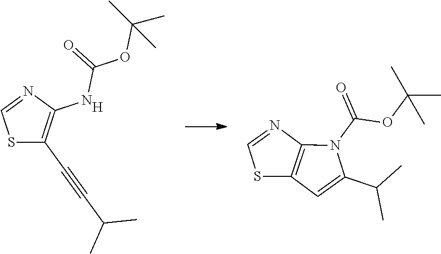

tert-Butyl N-[5-(3-methylbut-1-ynyl)thiazol-4-yl]carbamate (4 g, 15.017 mmol), copper(I) iodide (2.88 g, 15.122 mmol) and cesium carbonate (9.8 g, 30.078 mmol) were added into a high pressure tube, and N,N,N′,N′-Tetramethylethylenediamine (7.7600 g, 10 mL, 66.779 mmol) and THF (40 mL) were added. The tube was degassed with N₂ and stirred at 80° C. for 24 hours. The mixture was filtered through a Celite pad and washed with ethyl acetate (100 mL). The filtrate was evaporated under reduced pressure to afford a crude product which was purified by chromatography on silica gel (column: 40 g HP Gold; gradient: 2% ethyl acetate in heptane, 4 CV; 5 to 30%, ethyl acetate in heptane, 7 CV) to afford tert-butyl 5-isopropylpyrrolo[2,3-d]thiazole-4-carboxylate (2.3095 g, 58%) as light yellow solid. ¹H NMR (400 MHz, CDCl₃) δ 8.54 (s, 1H), 6.34-6.32 (m, 1H), 3.72-3.61 (m, 1H), 1.69 (s, 9H), 1.31 (d, J=6.7 Hz, 6H). ESI-MS m/z calc. 266.1089, found 211.2 (M−55)⁺; Retention time: 2.05 minutes; LC method I.

### Step 4: 5-Isopropyl-4H-pyrrolo[2,3-d]thiazole

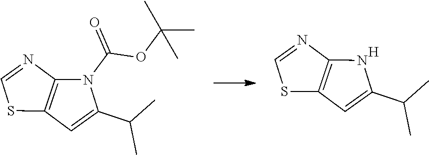

To a flame-dried flask (10 mL) was added tert-butyl 5-isopropylpyrrolo[2,3-d]thiazole-4-carboxylate (4.6 g, 17.253 mmol) and DCM (180 mL). The solution was stirred in an ice-saturated-NaCl-solution bath for 15 minutes. Then, TFA (8.8800 g, 6 mL, 77.879 mmol) was added dropwise using an additional funnel and stirred overnight (15 h). Supplementary TFA (88.800 g, 60 mL, 778.79 mmol) was added using an additional funnel over 1 hour while keeping the flask in an ice-saturated-NaCl-solution bath. The resulting solution was stirred for 5 hours. Then, the mixture was poured into an ice-water (200 mL) mixture. After that, the pH value of the mixture was adjusted with cold aqueous NaOH solution (6N) to 8-9. The layers were separated, and the organic layer was dried with Sodium sulfate, evaporated under reduced pressure and freeze-dried over the weekend to afford a crude 5-isopropyl-4H-pyrrolo[2,3-d]thiazole (2.6275 g, 92%) as a brown solid. ¹H NMR (400 MHz, CDCl₃) δ 9.56-9.20 (br. s, 1H), 8.43 (s, 1H), 6.22-6.08 (m, 1H), 3.18-3.02 (m, 1H), 1.37 (d, J=7.0 Hz, 6H). ESI-MS m/z calc. 166.05647, found 167.2 (M+1)⁺; Retention time: 1.7 minutes; LC method I.

### Step 5: 5-Isopropyl-4-methyl-pyrrolo[2,3-d]thiazole

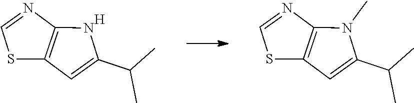

To a suspension of 5-isopropyl-4H-pyrrolo[2,3-d]thiazole (2.6 g, 15.624 mmol) in DMF (59 mL) in a flame-dried flask was added cesium carbonate (6.62 g, 20.318 mmol). The mixture was stirred in a ice-water bath for 20 minutes. Then, dimethylsulfate (3.0590 g, 2.3 mL, 24.252 mmol) was added dropwise. After that, the mixture was allowed to warm up to room temperature (in water bath) and stirred overnight (22 h). Water bath was removed and supplementary dimethylsulfate (1.1970 g, 0.9 mL, 9.4901 mmol) and cesium carbonate (3 g, 9.2076 mmol) were added. After 3 hours, the reaction mixture was quenched by adding cold water (100 mL). The resulting mixture was extracted by ethyl acetate (100 mL×3). The layers were separated, and the organic layers were combined and evaporated under reduced pressure to afford a crude product which was purified by chromatography on silica gel (column: 40 gram HP Gold; gradient: 2% ethyl acetate in heptane, 6 CV; 5%, 4 CV then 5% to 25%, 5 CV). The desired fractions were collected and concentrated to afford 5-isopropyl-4-methyl-pyrrolo[2,3-d]thiazole (1.53 g, 52%) as a yellow liquid. ¹H NMR (400 MHz, CDCl₃) δ 8.40 (s, 1H), 6.16 (s, 1H), 3.85 (s, 3H), 3.11-2.99 (m, 1H), 1.33 (d, J=6.8 Hz, 6H). ESI-MS m/z calc. 180.07211, found 181.2 (M+1)⁺; Retention time: 1.82 minutes; LC method I.

### Step 6: 5-Isopropyl-4-methyl-pyrrolo[2,3-d]thiazole-2-carbaldehyde

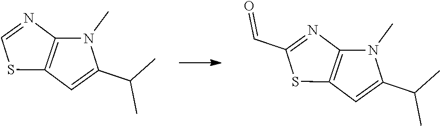

To a flask that containing 5-isopropyl-4-methyl-pyrrolo[2,3-d]thiazole (1.15 g, 6.0986 mmol) was added THF (41.5 mL). The solution was stirred at −78° C. for 20 minutes. Then, LHMDS in THF solution (10.8 mL of 1 M, 10.800 mmol) was added. The solution was stirred for 40 minutes, and DMF (2.5488 g, 2.7 mL, 34.870 mmol) was added. The solution was stirred for 3 hours. Then, the mixture was poured into cold sat. NH₄C₁ solution (50 mL) and resulting mixture was extracted with ethyl acetate (3×30 mL). The organic layers and the aqueous layer were separated. The organic layers were combined and evaporated under reduced pressure to afford a crude product. The crude was purified by chromatography on silica gel (column: 12 g HP Gold; gradient: 5% ethyl acetate in heptane, 5 CV; 5 to 30%, ethyl acetate in heptane, 12 CV) to afford 5-isopropyl-4-methyl-pyrrolo[2,3-d]thiazole-2-carbaldehyde (1.4 g, 38%) as a yellow solid. ¹H NMR (400 MHz, CDCl₃) δ 9.98-9.94 (m, 1H), 6.29-6.24 (m, 1H), 3.88 (s, 3H), 3.15-3.07 (m, 1H), 1.38-1.35 (m, 6H). ESI-MS m/z calc. 208.06703, found 209.2 (M+1)⁺; Retention time: 1.83 minutes; LC method I.

### Step 7: 3-[[4-(2,6-Dimethylphenyl)-6-[(2R)-2-[(5-isopropyl-4-methyl-pyrrolo[2,3-d]thiazol-2-yl)methylamino]-4,4-dimethyl-pentoxy]pyrimidin-2-yl]sulfamoyl]benzoic acid

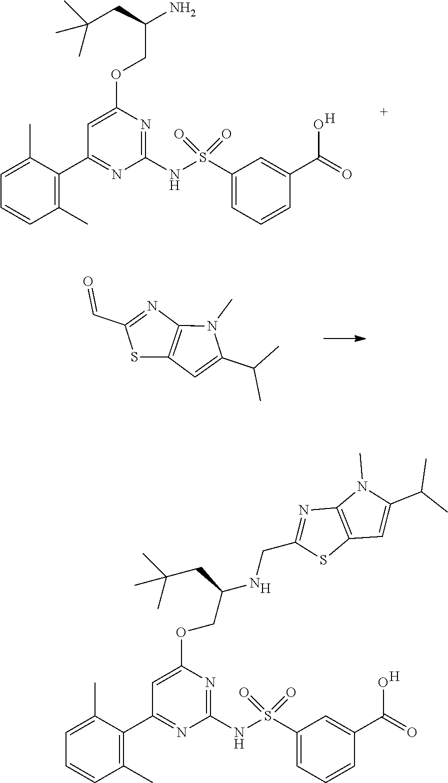

To a flame-dried flask (100 mL) were added 5-isopropyl-4-methyl-pyrrolo[2,3-d]thiazole-2-carbaldehyde (162 mg, 0.2668 mmol) and 3-[[4-[(2R)-2-amino-4,4-dimethyl-pentoxy]-6-(2,6-dimethylphenyl)pyrimidin-2-yl]sulfamoyl]benzoic acid (hydrochloride salt) (147 mg, 0.2677 mmol), and dichloromethane (26 mL). The solution was stirred in an ice-water bath. After 40 minutes, sodium triacetoxyborohydride (171 mg, 0.8068 mmol) was added. After another 40 minutes, supplementary sodium triacetoxyborohydride (80 mg, 0.3775 mmol) was added. After another 40 minutes, supplementary sodium triacetoxyborohydride (107 mg, 0.5049 mmol) was added. The solution was stirred at an ice-water bath overnight. The mixture was evaporated to afford a crude which was purified with reverse-phase chromatography on C₁₈ (column: 15.5 gram HP Gold; gradient: 5 to 100% acetonitrile in water containing 0.1% v/v of HCl acid, 18 CV) to afford 3-[[4-(2,6-dimethylphenyl)-6-[(2R)-2-[(5-isopropyl-4-methyl-pyrrolo[2,3-d]thiazol-2-yl)methylamino]-4,4-dimethyl-pentoxy]pyrimidin-2-yl]sulfamoyl]benzoic acid (hydrochloride salt) (94 mg, 43%) as a white powder. ¹H NMR (400 MHz, methanol-d4) δ 8.71-8.69 (m, 1H), 8.24-8.19 (m, 1H), 8.19-8.14 (m, 1H), 7.68-7.62 (m, 1H), 7.32-7.26 (m, 1H), 7.18-7.13 (m, 2H), 6.30 (s, 1H), 4.78-4.74 (m, 1H), 4.75-4.64 (m, 3H), 4.52-4.46 (m, 1H), 3.94-3.86 (m, 1H), 3.82-3.78 (m, 3H), 3.15-3.06 (m, 1H), 2.10 (s, 6H), 1.99-1.91 (m, 1H), 1.73-1.67 (m, 1H), 1.31 (d, J=6.8 Hz, 6H), 1.05 (s, 9H), −0.19-−0.34 (m, 3H). (2H missing, one labile proton; one is alkene proton probably due to overlap with water or D-H exchange) ESI-MS m/z calc. 704.28143, found 705.3 (M+1)⁺; Retention time: 1.65 minutes; LC method I.

### Step 8: (11R)-6-(2,6-Dimethylphenyl)-11-(2,2-dimethylpropyl)-12-[(5-isopropyl-4-methyl-pyrrolo[2,3-d]thiazol-2-yl)methyl]-2,2-dioxo-9-oxa-2λ⁶-thia-3,5,12,19-tetrazatricyclo[12.3.1.14,8]nonadeca-1(18),4(19),5,7,4,16-hexaen-13-one (Compound III-20

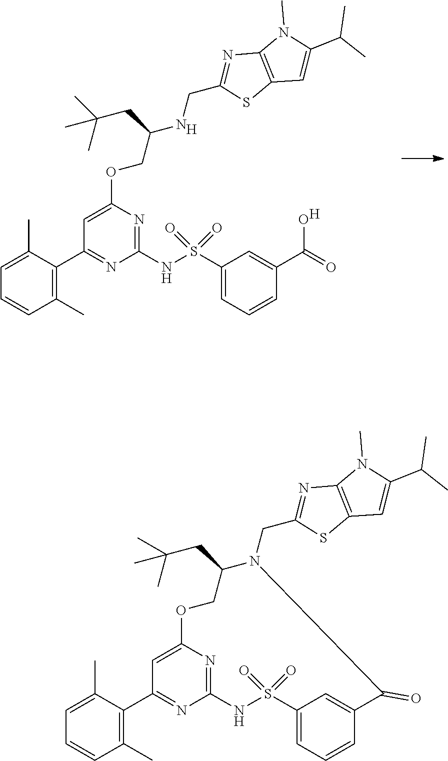

3-[[4-(2,6-Dimethylphenyl)-6-[(2R)-2-[(5-isopropyl-4-methyl-pyrrolo[2,3-d]thiazol-2-yl)methylamino]-4,4-dimethyl-pentoxy]pyrimidin-2-yl]sulfamoyl]benzoic acid (hydrochloride salt) (60 mg, 0.0766 mmol) was dissolved in DMF (0.7 mL) and ethyl acetate (5.4 mL) in a flask (25 mL). The solution was cooled down in an ice-water bath and stirred for 5 minutes. Then triethylamine (58.080 mg, 80 μL, 0.5740 mmol) was added. After another 5 minutes, propylphosphonic anhydride solution in ethyl acetate (181 mg, 50% w/w, 0.2844 mmol) was added. The reaction was stirred for 15 minutes at 0° C., then it was stirred at room temperature overnight (18 h). The reaction was washed with water (10 mL) and brine (10 mL), and the resulting solution was concentrated under reduced pressure to afford a bright yellow oil which was purified by reverse-phase chromatography on C₁₈ (column: 15.5 g HP Gold C₁₈; gradient: 30 to 100% acetonitrile in water containing 0.1% v/v of HCl; 18 CV). The volatiles were removed under reduced pressure and the remaining water was co-evaporated with methanol (3×10 mL). The residue was freeze-dried to afford (11R)-6-(2,6-dimethylphenyl)-11-(2,2-dimethylpropyl)-12-[(5-isopropyl-4-methyl-pyrrolo[2,3-d]thiazol-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (23 mg, 40%) as a pale-brown powder. ¹H NMR (400 MHz, CDCl₃) δ 8.75-8.69 (m, 1H), 8.19-8.11 (m, 1H), 7.95-7.88 (m, 1H), 7.75-7.66 (m, 1H), 7.26-7.20 (m, 1H), 7.11-7.05 (m, 2H), 6.32-6.28 (m, 1H), 6.14 (s, 1H), 5.47 (d, J=15.5 Hz, 1H), 5.33-5.24 (m, 1H), 4.74-4.66 (m, 1H), 4.43-4.33 (m, 1H), 4.19-4.09 (m, 1H), 3.87 (s, 3H), 3.09-3.00 (m, 1H), 2.05 (br. s, 6H), 1.95-1.84 (m, 1H), 1.61-1.52 (m, 1H), 1.35-1.31 (m, 6H), 0.61 (s, 9H). (2H missing, labile protons) ESI-MS m/z calc. 686.2709, found 686.9 (M+1)⁺; Retention time: 5.1 minutes; LC method J.

### Example 72: Preparation of Compound I-134

### Step 1: 5-Chloro-3-(2-cyclobutylethynyl)-1-[(4-methoxyphenyl)methyl]pyrazin-2-one

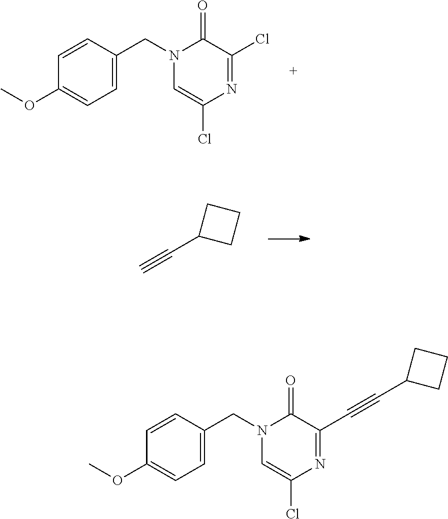

To a 20 mL microwave vial was charged 3,5-dichloro-1-[(4-methoxyphenyl)methyl]pyrazin-2-one (575 mg, 2.017 mmol), bis(triphenylphosphine) palladium(II) dichloride (26 mg, 0.03704 mmol), CuI (18 mg, 0.09451 mmol) in DMF (4.5 mL) and TEA (1.7 mL, 12.20 mmol). Then, ethynylcyclobutane (245 mg, 3.058 mmol) was added and the vessel was sealed. The mixture was irradiated at 80° C. μW for 10 min. The reaction mixture was allowed to cool, diluted with DCM (20 mL) and washed with water (2×10 mL). The organic layer was washed with brine (10 mL), dried with sodium sulfate, filtered and concentrated under vacuo. The resulting material was purified by silica gel chromatography (24 g of silica) using a gradient eluent of 100% hexanes to 80% ethyl acetate in hexanes to afford as a brown gum 5-chloro-3-(2-cyclobutylethynyl)-1-[(4-methoxyphenyl)methyl]pyrazin-2-one (621.2 mg, 94%). ESI-MS m/z calc. 328.09787, found 329.3 (M+1)⁺; Retention time: 1.88 minutes. LC method A.

### Step 2: 2-Chloro-6-cyclobutyl-furo[2,3-b]pyrazine

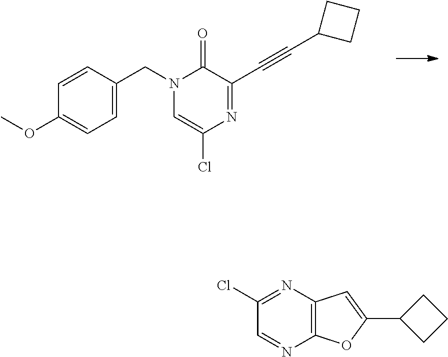 [SMILES: COc1ccc(Cn2cc(Cl)nc(C#CC3CCC3)c2=O)cc1 ; Clc1cnc2oc(C3CCC3)cc2n1]

To a solution of 5-chloro-3-(2-cyclobutylethynyl)-1-[(4-methoxyphenyl)methyl]pyrazin-2-one (621 mg, 1.889 mmol) in DCM (12 mL), Trifluoromethanesulfonate (25 mg, 0.09730 mmol) (silver salt) and TFA (1.3 mL, 16.87 mmol) were added and stirred at room temperature for 60 minutes. The residue was concentrated in vacuo and then purified by silica gel chromatography (40 g of silica) using a gradient eluent of 100% hexanes to 30% ethyl acetate in hexanes to give as an off-white solid 2-chloro-6-cyclobutyl-furo[2,3-b]pyrazine (230 mg, 58%), ¹H NMR (400 MHz, DMSO-d6) δ 8.37 (s, 1H), 7.05 (s, 1H), 3.86-3.79 (m, 1H), 2.42-2.28 (m, 4H), 2.14-2.02 (m, 1H), 2.00-1.89 (m, 1H). ESI-MS m/z calc. 208.04034, found 209.2 (M+1)⁺; Retention time: 1.85 minutes. LC method A.

### Step 3: Methyl 6-cyclobutylfuro[2,3-b]pyrazine-2-carboxylate

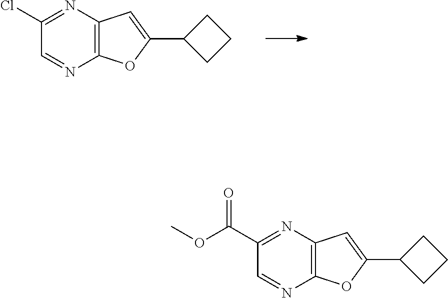 [SMILES: Clc1cnc2oc(C3CCC3)cc2n1 ; COC(=O)c1cnc2oc(C3CCC3)cc2n1]

A mixture of 2-chloro-6-cyclobutyl-furo[2,3-b]pyrazine (220 mg, 1.054 mmol) and Pd(dppf)Cl₂·DCM (88 mg, 0.1078 mmol) and TEA (1000 μL, 7.175 mmol) in MeOH (9 mL) in a steel pressure vessel equipped with a stir bar was purged with carbon monoxide (3 g, 103.4 mmol) three times. The reaction mixture was heated to 100° C. with 120 psi of carbon monoxide and stirred for 14 h. The reaction mixture was allowed to cool to rt. The reaction mixture was filtered through a pad of celite, washed with ethyl acetate, filtrate was concentrated in vacuo. The residue was purified by silica gel chromatography (40 g of silica) using a gradient eluent of 100% hexanes to 50% ethyl acetate in hexanes to give as a white solid methyl 6-cyclobutylfuro[2,3-b]pyrazine-2-carboxylate (120.6 mg, 49%), ¹H NMR (400 MHz, Chloroform-d) δ 9.01 (s, 1H), 6.73 (s, 1H), 4.06 (s, 3H), 3.82-3.72 (m, 1H), 2.50-2.38 (m, 4H), 2.20-2.00 (m, 2H). ESI-MS m/z calc. 232.0848, found 233.2 (M+1)⁺; Retention time: 1.46 minutes. LC method A.

### Step 4: 6-Cyclobutylfuro[2,3-b]pyrazine-2-carbaldehyde

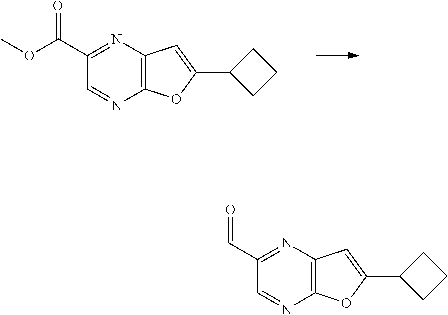 [SMILES: COC(=O)c1cnc2oc(C3CCC3)cc2n1 ; O=Cc1cnc2oc(C3CCC3)cc2n1]

To a stirred solution of methyl 6-cyclobutylfuro[2,3-b]pyrazine-2-carboxylate (118 mg, 0.5081 mmol) in DCM (4.0 mL) was added DIBAL (in hexanes) (1.2 mL of 1 M, 1.200 mmol) at −78° C. over 2 minutes. The reaction mixture was stirred for 1 h, quenched with MeOH (3 mL)/water (3 mL) and concentrated in vacuo. DCM (10 mL) was added and filtered. The cake was rinsed with DCM. The organic layer from the filtrate was separated, washed with brine (2 mL), dried over sodium sulfate, filtered, and concentrated in vacuo. The residue was purified by silica gel chromatography (24 g of silica) by using 0-50% ethyl acetate in hexanes to afford 6-cyclobutylfuro[2,3-b]pyrazine-2-carbaldehyde (73.6 mg, 72%), ¹H NMR (400 MHz, Chloroform-d) δ 10.20 (s, 1H), 8.86 (s, 1H), 6.75 (d, J=0.8 Hz, 1H), 3.85-3.72 (m, 1H), 2.55-2.37 (m, 4H), 2.24-1.99 (m, 2H). ESI-MS m/z calc. 202.07423, found 203.2 (M+1)⁺; Retention time: 1.44 minutes. LC method A.

### Step 5: 3-[[4-[(2R)-3-(1-Bicyclo[1.1.1]pentanyl)-2-[(6-cyclobutylfuro[2,3-b]pyrazin-2-yl)methylamino]propoxy]-6-(2,6-dimethylphenyl)pyrimidin-2-yl]sulfamoyl]benzoic acid

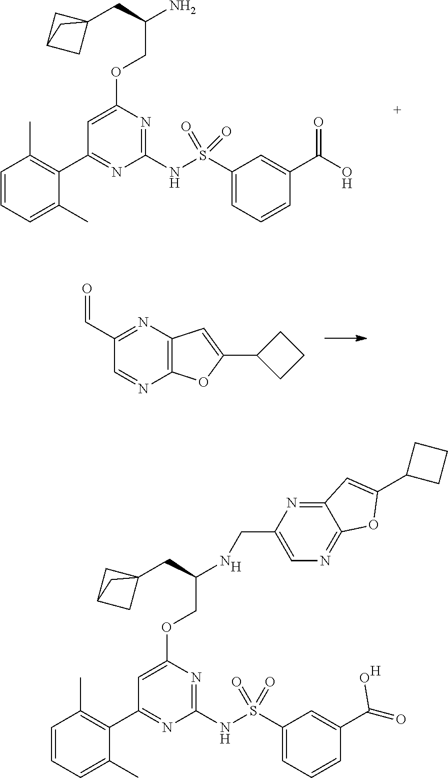

A 20 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (63 mg, 0.1120 mmol), 6-cyclobutylfuro[2,3-b]pyrazine-2-carbaldehyde (23.6 mg, 0.1167 mmol), anhydrous DCM (1,500 μL), acetic acid (16 μL, 0.2814 mmol) and DIEA (83 μL, 0.4765 mmol). The mixture was cooled down in an ice bath, sodium triacetoxyborohydride (90 mg, 0.4246 mmol) was added and the reaction was stirred for 3 h allowing the mixture to warm to room temperature. The reaction was quenched with aqueous 1 M HCl (1 mL), MeOH (0.5 mL) and DMSO (0.5 mL), filtered and purified by reverse phase HPLC (1-70% acetonitrile/5 mM aqueous HCl over 30 min) to give as a white solid 3-[[4-[(2R)-3-(1-bicyclo[1.1.1]pentanyl)-2-[(6-cyclobutylfuro[2,3-b]pyrazin-2-yl)methylamino]propoxy]-6-(2,6-dimethylphenyl)pyrimidin-2-yl]sulfamoyl]benzoic acid (hydrochloride salt) (26.5 mg, 32%). ESI-MS m/z calc. 708.273, found 709.6 (M+1)⁺; Retention time: 1.59 minutes. LC method A.

### Step 6: (11R)-11-(1-Bicyclo[1.1.1]pentanylmethyl)-12-[(6-cyclobutylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-134

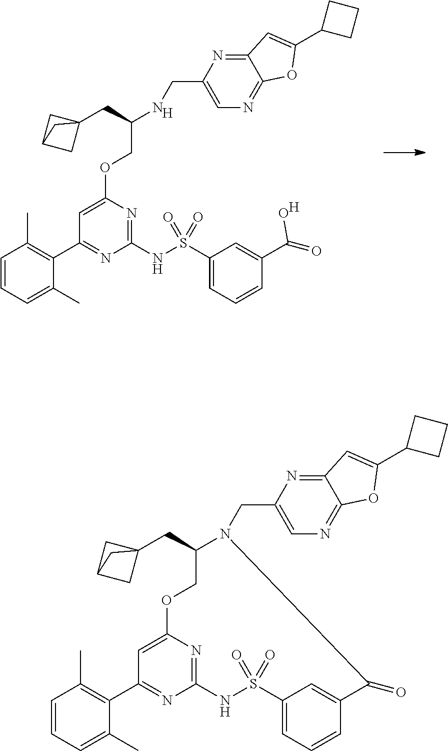

3-[[4-[(2R)-3-(1-Bicyclo[1.1.1]pentanyl)-2-[(6-cyclobutylfuro[2,3-b]pyrazin-2-yl)methylamino]propoxy]-6-(2,6-dimethylphenyl)pyrimidin-2-yl]sulfamoyl]benzoic acid (hydrochloride salt) (26.5 mg, 0.03556 mmol) was combined under nitrogen with CDMT (18 mg, 0.1025 mmol) and DMF (1.25 mL). The solution was stirred at 0° C. 4-Methyl-morpholine (26 μL, 0.2365 mmol) was added and the mixture was stirred in the cooling bath that was allowed to warm to room temperature. After 2 h, the reaction was filtered and purified by reverse phase HPLC (1-99% acetonitrile/5 mM aqueous HCl over 30 min) to give as a white solid (11R)-11-(1-bicyclo[1.1.1]pentanylmethyl)-12-[(6-cyclobutylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (22.6 mg, 91%), ¹H NMR (400 MHz, Chloroform-d) δ 8.72 (t, J=1.8 Hz, 1H), 8.40 (s, 1H), 8.15 (d, J=7.7 Hz, 1H), 7.91 (dt, J=7.7, 1.4 Hz, 1H), 7.69 (t, J=7.8 Hz, 1H), 7.22 (d, J=7.6 Hz, 1H), 7.06 (d, J=7.6 Hz, 2H), 6.65 (d, J=0.8 Hz, 1H), 6.25 (s, 1H), 5.44 (dd, J=11.4, 4.1 Hz, 1H), 5.33 (d, J=15.0 Hz, 1H), 4.31-4.19 (m, 2H), 4.07-3.97 (m, 1H), 3.80-3.67 (m, 1H), 2.48-2.37 (m, 5H), 2.15-2.08 (m, 1H), 2.06 (s, 6H), 2.04-1.95 (m, 2H), 1.80 (dd, J=15.5, 3.3 Hz, 1H), 1.63-1.53 (m, 6H) plus 1H not observed. ESI-MS m/z calc. 690.26245, found 691.5 (M+1)⁺; Retention time: 2.29 minutes. LC method A.

### Example 73: Preparation of Compound III-21

### Step 1: Methyl 6-chloro-3-methyl-pyrazine-2-carboxylate

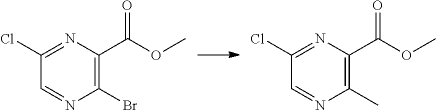

A mixture of methyl 3-bromo-6-chloro-pyrazine-2-carboxylate (2.01 g, 7.9932 mmol), trimethylboroxine (622.31 mg, 0.7 mL, 4.9077 mmol), potassium carbonate (110.2 mg, 0.7974 mmol) in DME (50 mL) was bubbled with nitrogen for 10 minutes. Pd(dppf)Cl₂·DCM (670.5 mg, 0.8211 mmol) was added to the reaction mixture. The reaction mixture was heated at 80° C. for 16 hours. After cooling to rt, trimethylboroxine (718.40 mg, 0.8 mL, 5.7227 mmol), Pd(dppf)Cl₂·DCM (670 mg, 0.8204 mmol) and potassium carbonate (1.02 g, 7.3803 mmol) were added to the reaction mixture. The reaction mixture was heated at 80° C. for 22 hours. After cooling to rt, the reaction mixture was filtered over Celite, and then rinsed with ethyl acetate (50 mL). The filtrate was concentrated under reduced pressure. The residue was purified by silica gel chromatography using 0 to 25% EtOAc in hexane to furnish methyl 6-chloro-3-methyl-pyrazine-2-carboxylate (677.4 mg, 46%) as light pink solid. ESI-MS m/z calc. 186.0196, found 187.2 (M+1)⁺; Retention time: 2.04 minutes; LC method E.

### Step 2: Methyl 3-(bromomethyl)-6-chloro-pyrazine-2-carboxylate

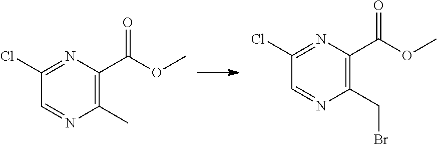

A mixture of methyl 6-chloro-3-methyl-pyrazine-2-carboxylate (3 g, 16.078 mmol), NBS (3.2 g, 17.979 mmol) and AIBN (396.4 mg, 2.4140 mmol) in CCl₄ (30 mL) was stirred at 80° C. for 2 h. NBS (1.51 g, 8.4839 mmol) and AIBN (230 mg, 1.4007 mmol) were added to the reaction mixture. The reaction mixture was stirred at 80° C. for 2 h. After cooling to room temperature, the reaction mixture was filtered. The filtrate was concentrated. The residue was purified by silica gel chromatography using 0-20% EtOAc in hexane to afford methyl 3-(bromomethyl)-6-chloro-pyrazine-2-carboxylate (4.0352 g, 62%) as white cloudy oil. ¹H NMR (400 MHz, Chloroform-d) δ 8.70 (s, 1H), 5.00 (s, 2H), 4.05 (s, 3H). ESI-MS m/z calc. 263.9301, found 265.8 (M+1)⁺; Retention time: 2.3 minutes; LC method E.

### Step 3: 3-Chloro-6-isopropyl-7H-pyrrolo[3,4-b]pyrazin-5-one

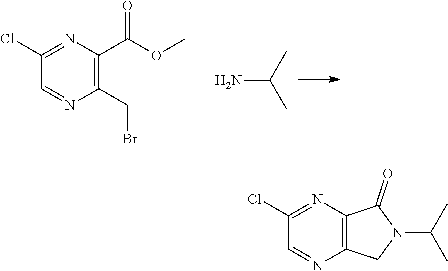

To a solution of methyl 3-(bromomethyl)-6-chloro-pyrazine-2-carboxylate (3.94 g, 9.7946 mmol) in MeCN (12.5 mL) was added propan-2-amine (8.6000 g, 12.5 mL, 145.49 mmol) and the reaction vessel was sealed. The microwave vial was irradiated at 80° C. for 0.5 h. The reaction mixture was cooled down to room temperature and concentrated in vacuo. The residue was purified by silica gel chromatography using 0-50% ethyl acetate in hexanes to afford 3-chloro-6-isopropyl-7H-pyrrolo[3,4-b]pyrazin-5-one (1.823 g, 88%) as a pale yellow solid. ¹H NMR (400 MHz, Chloroform-d) δ 8.68 (s, 1H), 4.81 (hept, J=6.7 Hz, 1H), 4.44 (s, 2H), 1.35 (d, J=7.0 Hz, 6H). ESI-MS m/z calc. 211.05124, found 211.9 (M+0)⁺; Retention time: 2.31 minutes; LC method D.

### Step 4: 6-Isopropyl-3-vinyl-7H-pyrrolo[3,4-b]pyrazin-5-one

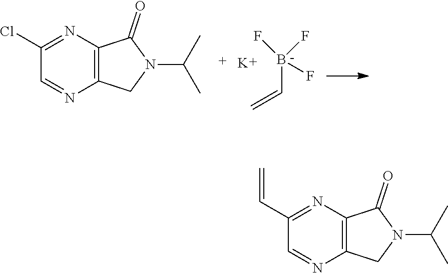

A mixture of 3-chloro-6-isopropyl-7H-pyrrolo[3,4-b]pyrazin-5-one (1.1 g, 5.1973 mmol), potassium vinyltrifluoroborate (1.76 g, 13.139 mmol), Pd(dppf)Cl₂ (297.1 mg, 0.3638 mmol) and TEA (2.4684 g, 3.4 mL, 24.394 mmol) in EtOH (95 mL) was degassed and heated in a sealed vial at 90° C. for 1 h. The solvent was evaporated. The residue was purified by silica gel chromatography by using 0-70% EtOAc in hexane to afford 6-isopropyl-3-vinyl-7H-pyrrolo[3,4-b]pyrazin-5-one (1.001 g, 95%) as a yellow solid. ¹H NMR (500 MHz, Chloroform-d) δ 8.67 (s, 1H), 6.96 (dd, J=16.5, 11.2 Hz, 1H), 6.54 (d, J=17.3 Hz, 1H), 5.71 (d, J=10.5 Hz, 1H), 4.84 (d, J=6.8 Hz, 1H), 4.41 (s, 2H), 1.34 (d, J=6.8 Hz, 6H). ESI-MS m/z calc. 203.10587, found 204.5 (M+1)⁺; Retention time: 1.77 minutes; LC method E.

### Step 5: 6-Isopropyl-5-oxo-7H-pyrrolo[3,4-b]pyrazine-3-carbaldehyde

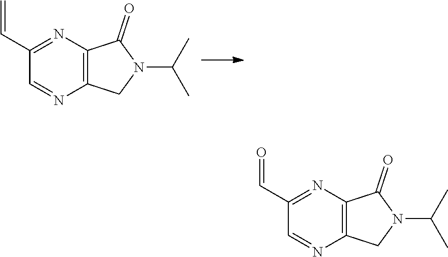

To a solution of 6-isopropyl-3-vinyl-7H-pyrrolo[3,4-b]pyrazin-5-one (1 g, 4.6743 mmol) in dioxane (15 mL) and water (5 mL) was added pyridine (746.90 mg, 0.77 mL, 9.4425 mmol), OsO₄ in tBuOH (7.2 mL of 2.5% w/v, 0.7080 mmol) then NaIO₄ (4 g, 18.701 mmol) at room temperature. The reaction was stirred at room temperature for 2 h. The reaction was diluted with DCM (40 mL) washed with NaHCO₃ (40 mL), extracted with DCM (3×30 mL), washed with brine (40 mL) dried over Na₂SO₄, filtered and concentrated in vacuo. The residue was purified by flash chromatography using 20-100% EtOAc in Hexanes to provide 6-isopropyl-5-oxo-7H-pyrrolo[3,4-b]pyrazine-3-carbaldehyde (128.6 mg, 10%) as a light brown gel. ¹H NMR (500 MHz, Chloroform-d) δ 10.30 (s, 1H), 9.27 (s, 1H), 4.86 (hept, J=6.8 Hz, 1H), 4.55 (s, 2H), 1.38 (d, J=6.8 Hz, 6H). ESI-MS m/z calc. 205.08513, found 206.2 (M+1)⁺; Retention time: 0.88 minutes; LC method H.

### Step 6: 3-[[4-(2,6-Dimethylphenyl)-6-[(2R)-2-[(6-isopropyl-5-oxo-7H-pyrrolo[3,4-b]pyrazin-3-yl)methylamino]-3-(1-methylcyclopropyl)propoxy]pyrimidin-2-yl]sulfamoyl]benzoic acid

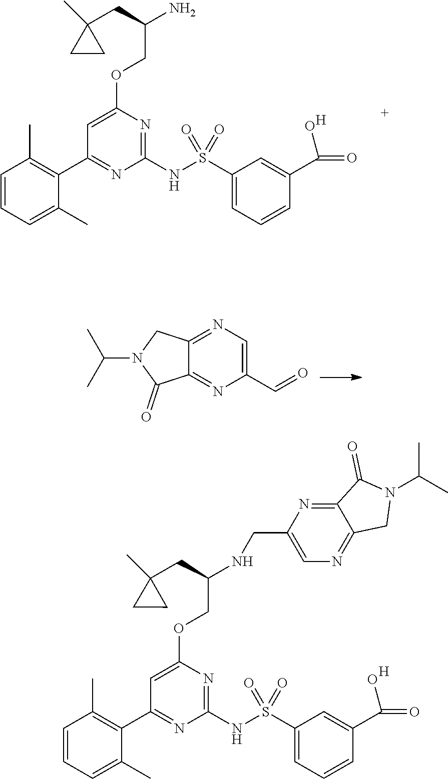

A 4 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (90 mg, 0.1645 mmol), 6-isopropyl-5-oxo-7H-pyrrolo[3,4-b]pyrazine-3-carbaldehyde (45.6 mg, 0.1647 mmol), anhydrous DCM (0.82 mL), and acetic acid (9.6 μL, 0.1688 mmol). The mixture was cooled down in an ice bath. DIEA (57.40 μL, 0.3295 mmol) was added, followed by sodium triacetoxyborohydride (182.5 mg, 0.8611 mmol), and the reaction was vigorously stirred at 0° C. for 6 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-(2,6-dimethylphenyl)-6-[(2R)-2-[(6-isopropyl-5-oxo-7H-pyrrolo[3,4-b]pyrazin-3-yl)methylamino]-3-(1-methylcyclopropyl)propoxy]pyrimidin-2-yl]sulfamoyl]benzoic acid (hydrochloride salt) (7.4 mg, 6%) as a white solid. ESI-MS m/z calc. 699.2839, found 700.2 (M+1)⁺; Retention time: 1.17 minutes. LC method A.

### Step 7: (11R)-6-(2,6-Dimethylphenyl)-12-[(6-isopropyl-5-oxo-7H-pyrrolo[3,4-b]pyrazin-3-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound III-21

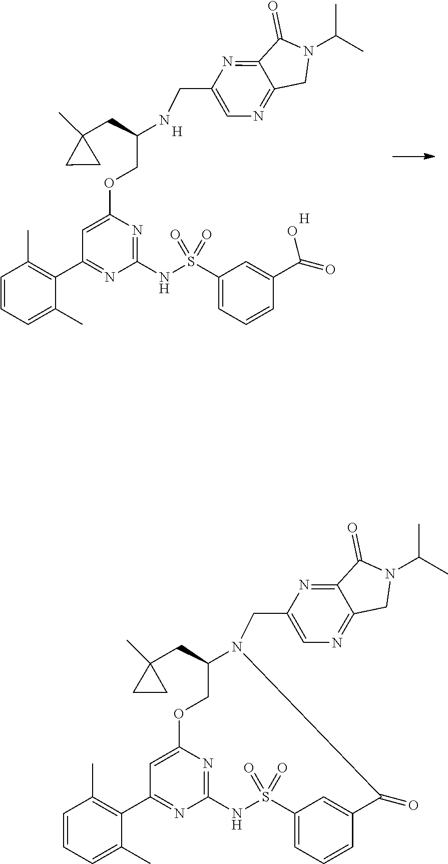

3-[[4-(2,6-Dimethylphenyl)-6-[(2R)-2-[(6-isopropyl-5-oxo-7H-pyrrolo[3,4-b]pyrazin-3-yl)methylamino]-3-(1-methylcyclopropyl)propoxy]pyrimidin-2-yl]sulfamoyl]benzoic acid (hydrochloride salt) (7.4 mg, 0.009045 mmol) was combined with CDMT (5.7 mg, 0.03247 mmol) in DMF (1.0 mL) and cooled to 0° C. N-methylmorpholine (5.7 μL, 0.05185 mmol) was added by syringe and the reaction was stirred at 0° C. for 30 minutes. The ice bath was then removed and stirring was continued for an additional 16 hours at room temperature. The reaction mixture was then partitioned between 50 mL 1 M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-6-(2,6-dimethylphenyl)-12-[(6-isopropyl-5-oxo-7H-pyrrolo[3,4-b]pyrazin-3-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (2.3 mg, 31%) as a white solid. ¹H NMR (400 MHz, CDCl₃) δ 9.06 (s, 1H), 8.55-8.51 (m, 1H), 8.13 (d, J=7.9 Hz, 1H), 7.86 (d, J=7.5 Hz, 1H), 7.65 (t, J=7.8 Hz, 1H), 7.21 (t, J=7.6 Hz, 1H), 7.06 (d, J=7.6 Hz, 2H), 6.22 (s, 1H), 5.38 (d, J=14.4 Hz, 1H), 5.08 (dd, J=10.5, 3.7 Hz, 1H), 4.83 (hept, J=6.7 Hz, 1H), 4.55-4.38 (m, 4H), 4.26 (d, J=14.4 Hz, 1H), 2.01 (s, 6H), 1.80 (d, J=15.2 Hz, 1H), 1.57-1.50 (m, 1H), 1.38 (d, J=3.3 Hz, 3H), 1.36 (d, J=3.3 Hz, 3H), 0.46 (s, 3H), 0.35-0.27 (m, 1H), 0.27-0.18 (m, 1H), 0.16-0.10 (m, 1H), 0.04-0.01 (m, 1H). ESI-MS m/z calc. 681.2733, found 682.1 (M+1)⁺; Retention time: 1.6 minutes. LC method A.

### Example 74: Preparation of Compound I-143

### Step 1: 5-Chloro-1-[(4-methoxyphenyl)methyl]-3-[2-(1-methylcyclobutyl)ethynyl]pyrazin-2-one

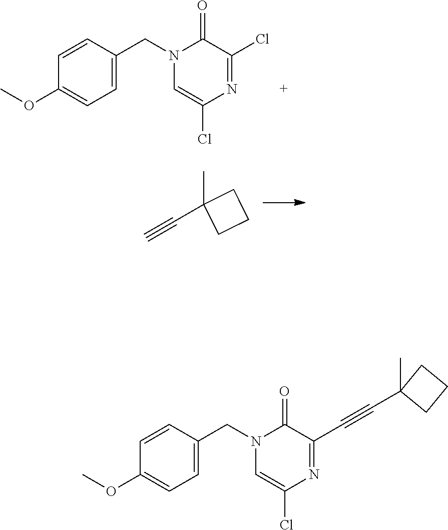

To a 20 mL microwave vial was charged 3,5-dichloro-1-[(4-methoxyphenyl)methyl]pyrazin-2-one (950 mg, 3.332 mmol), bis(triphenylphosphine)palladium(II) dichloride (45 mg, 0.06411 mmol), CuI (30 mg, 0.1575 mmol) in DMF (5 mL) and TEA (2.5 mL, 17.94 mmol). Then, 1-ethynyl-1-methyl-cyclobutane (476 mg, 5.056 mmol) was added and the vessel was sealed. The mixture was irradiated at 80° C. μW for 10 min. The reaction mixture was allowed to cool, diluted with DCM (20 mL) and washed with water (2×10 mL). The organic layer was washed with brine (10 mL), dried with sodium sulfate, filtered, and concentrated in vacuo. The resulting material was purified by silica gel chromatography (40 g of silica) using a gradient eluent of 100% hexanes to 80% ethyl acetate in hexanes to afford as a brown gum 5-chloro-1-[(4-methoxyphenyl)methyl]-3-[2-(1-methylcyclobutyl)ethynyl]pyrazin-2-one (1.05 g, 92%), ¹H NMR (400 MHz, Methanol-d4) δ 7.78 (s, 1H), 7.37-7.31 (m, 2H), 6.95-6.90 (m, 2H), 5.05 (s, 2H), 3.78 (s, 3H), 2.55-2.39 (m, 2H), 2.15-2.01 (m, 4H), 1.49 (s, 3H). ESI-MS m/z calc. 342.1135, found 343.2 (M+1)⁺; Retention time: 1.99 minutes. LC method A.

### Step 2: 2-Chloro-6-(1-methylcyclobutyl)furo[2,3-b]pyrazine

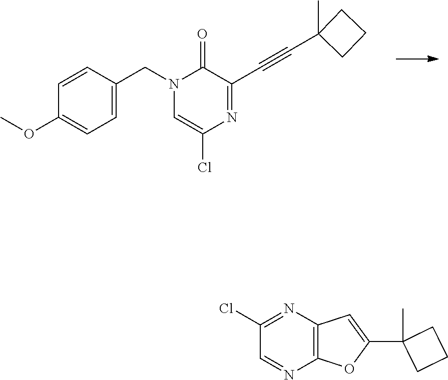 [SMILES: COc1ccc(Cn2cc(Cl)nc(C#CC3(C)CCC3)c2=O)cc1 ; CC1(c2cc3nc(Cl)cnc3o2)CCC1]

To a solution of 5-chloro-1-[(4-methoxyphenyl)methyl]-3-[2-(1-methylcyclobutyl)ethynyl]pyrazin-2-one (1.05 g, 3.063 mmol) in DCM (20.0 mL), trifluoromethanesulfonate (41 mg, 0.1596 mmol) (silver salt) and TFA (2.2 mL, 28.56 mmol) were added and stirred at room temperature for 60 minutes. The residue was concentrated in vacuo and then purified by silica gel chromatography (80 g of silica) using a gradient eluent of 100% hexanes to 30% ethyl acetate in hexanes to give as a white solid 2-chloro-6-(1-methylcyclobutyl)furo[2,3-b]pyrazine (612 mg, 90%), ¹H NMR (400 MHz, Methanol-d4) δ 8.21 (s, 1H), 6.76 (s, 1H), 2.66-2.56 (m, 2H), 2.21-2.01 (m, 4H), 1.63 (s, 3H). ESI-MS m/z calc. 222.05598, found 223.2 (M+1)⁺; Retention time: 1.99 minutes. LC method A.

### Step 3: Methyl 6-(1-methylcyclobutyl)furo[2,3-b]pyrazine-2-carboxylate

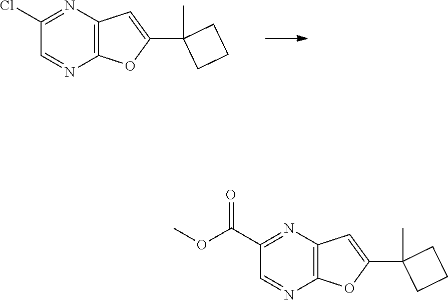 [SMILES: CC1(c2cc3nc(Cl)cnc3o2)CCC1 ; COC(=O)c1cnc2oc(C3(C)CCC3)cc2n1]

A mixture of 2-chloro-6-(1-methylcyclobutyl)furo[2,3-b]pyrazine (608 mg, 2.730 mmol) and Pd(dppf)Cl₂·DCM (229 mg, 0.2804 mmol) and TEA (2.75 mL, 19.73 mmol) in MeOH (25 mL) in a steel pressure vessel equipped with a stir bar was purged with carbon monoxide (7.75 g, 267.1 mmol) three times. The reaction mixture was heated to 100° C. with 120 psi of carbon monoxide and stirred for 18 h. The reaction mixture was allowed to cool to rt. The reaction mixture was filtered through a pad of celite, washed with ethyl acetate, filtrate was concentrated in vacuo. The residue was purified by silica gel chromatography (40 g of silica) using a gradient eluent of 100% hexanes to 50% ethyl acetate in hexanes to give as a white solid methyl 6-(1-methylcyclobutyl)furo[2,3-b]pyrazine-2-carboxylate (556.7 mg, 83%), ¹H NMR (400 MHz, Chloroform-d) δ 9.01 (s, 1H), 6.71 (s, 1H), 4.06 (s, 3H), 2.69-2.54 (m, 2H), 2.23-1.99 (m, 4H), 1.65 (s, 3H). ESI-MS m/z calc. 246.10045, found 247.2 (M+1)⁺; Retention time: 1.65 minutes. LC method A.

### Step 4: 6-(1-Methylcyclobutyl)furo[2,3-b]pyrazine-2-carbaldehyde

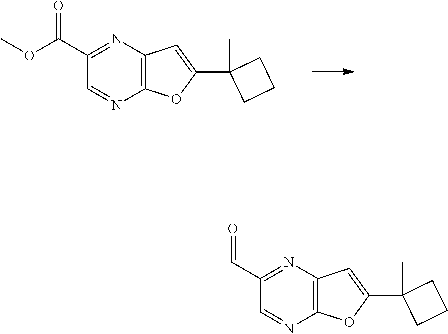 [SMILES: COC(=O)c1cnc2oc(C3(C)CCC3)cc2n1 ; CC1(c2cc3nc(C=O)cnc3o2)CCC1]

To a stirred solution of methyl 6-(1-methylcyclobutyl)furo[2,3-b]pyrazine-2-carboxylate (553.5 mg, 2.248 mmol) in DCM (20 mL) was added DIBAL (in hexanes) (5.5 mL of 1 M, 5.500 mmol) at −78° C. over 2 minutes. The reaction mixture was stirred for 1 h, quenched with MeOH (10 mL)/water (10 mL) and concentrated in vacuo. DCM (20 mL) was added and filtered. The cake was rinsed with DCM. The organic layer from the filtrate was separated, washed with brine (2 mL), dried over sodium sulfate, filtered, and concentrated in vacuo. The residue was purified by silica gel chromatography (40 g of silica) by using 0-50% ethyl acetate in hexanes to afford 6-(1-methylcyclobutyl)furo[2,3-b]pyrazine-2-carbaldehyde (265 mg, 55%), ¹H NMR (400 MHz, Chloroform-d) δ 10.20 (s, 1H), 8.87 (s, 1H), 6.74 (s, 1H), 2.67-2.57 (m, 2H), 2.20-2.03 (m, 4H), 1.66 (s, 3H). ESI-MS m/z calc. 216.08987, found 217.2 (M+1)⁺; Retention time: 1.6 minutes. LC method A.

### Step 5: 3-[[4-(2,6-Dimethylphenyl)-6-[(2R)-2-[[6-(1-methylcyclobutyl)furo[2,3-b]pyrazin-2-yl]methylamino]-3-(1-methylcyclopropyl)propoxy]pyrimidin-2-yl]sulfamoyl]benzoic acid

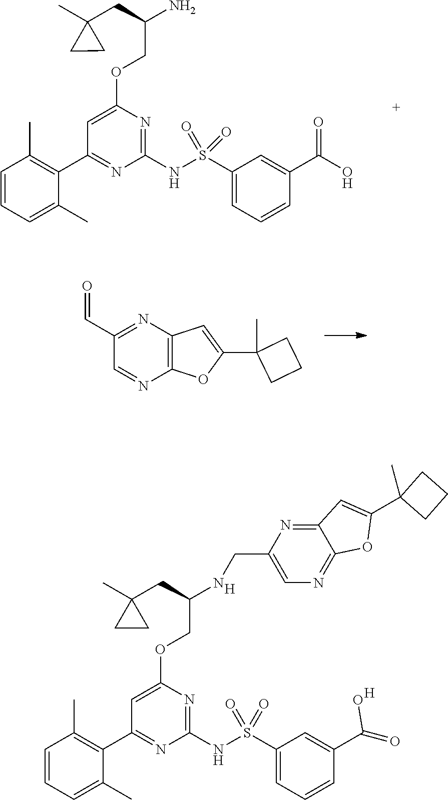

A 20 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (68 mg, 0.1239 mmol), 6-(1-methylcyclobutyl)furo[2,3-b]pyrazine-2-carbaldehyde (28 mg, 0.1295 mmol), anhydrous DCM (2.5 mL), acetic acid (20 μL, 0.3517 mmol) and DIEA (90 μL, 0.5167 mmol). The mixture was cooled down in an ice bath, sodium triacetoxyborohydride (100 mg, 0.4718 mmol) was added and the reaction was stirred and allowed to warm to rt gradually over 4 h. The reaction was quenched with aqueous 1 M HCl (1 mL), MeOH (0.5 mL) and DMSO (0.5 mL), filtered and purified by reverse phase HPLC (1-70% acetonitrile/5 mM aqueous HCl over 30 min) to give as a white solid 3-[[4-(2,6-dimethylphenyl)-6-[(2R)-2-[[6-(1-methylcyclobutyl)furo[2,3-b]pyrazin-2-yl]methylamino]-3-(1-methylcyclopropyl)propoxy]pyrimidin-2-yl]sulfamoyl]benzoic acid (hydrochloride salt) (67.3 mg, 73%). ESI-MS m/z calc. 710.28864, found 711.4 (M+1)⁺; Retention time: 1.62 minutes. LC method A.

### Step 6: (11R)-6-(2,6-Dimethylphenyl)-12-[[6-(1-methylcyclobutyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-143

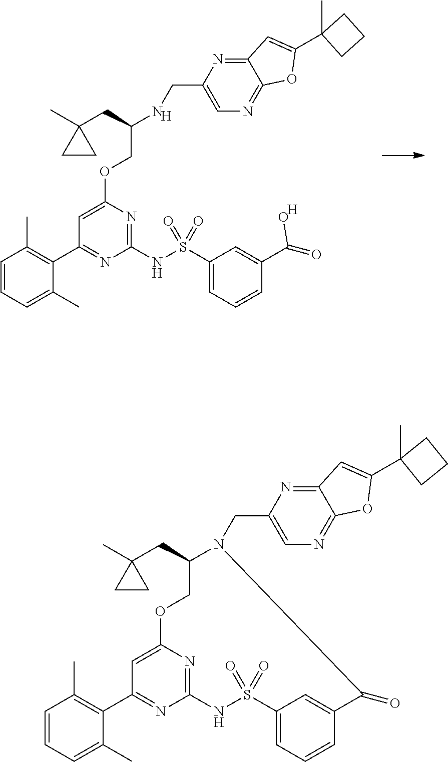

3-[[4-(2,6-Dimethylphenyl)-6-[(2R)-2-[[6-(1-methylcyclobutyl)furo[2,3-b]pyrazin-2-yl]methylamino]-3-(1-methylcyclopropyl)propoxy]pyrimidin-2-yl]sulfamoyl]benzoic acid (hydrochloride salt) (67 mg, 0.08966 mmol) was combined under nitrogen with CDMT (45 mg, 0.2563 mmol) and DMF (2.00 mL). The solution was stirred at 0° C. 4-Methyl-morpholine (75 μL, 0.6822 mmol) was added and the mixture was stirred in the cooling bath that was allowed to warm to room temperature. After 16 h, the reaction was filtered and purified by reverse phase HPLC (1-99% acetonitrile/5 mM aqueous HCl over 30 min) to give as a white solid (11R)-6-(2,6-dimethylphenyl)-12-[[6-(1-methylcyclobutyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (35.3 mg, 56%), ¹H NMR (400 MHz, CDCl₃) δ 8.73 (t, J=1.8 Hz, 1H), 8.41 (s, 1H), 8.11-8.04 (m, 1H), 7.87 (dt, J=7.7, 1.4 Hz, 1H), 7.63 (t, J=7.8 Hz, 1H), 7.21 (t, J=7.6 Hz, 1H), 7.06 (d, J=7.6 Hz, 2H), 6.64 (s, 1H), 6.25 (s, 1H), 5.40 (dd, J=11.2, 4.1 Hz, 1H), 5.29 (d, J=14.8 Hz, 1H), 4.42 (t, J=11.3 Hz, 1H), 4.30 (t, J=11.5 Hz, 1H), 4.16 (d, J=14.8 Hz, 1H), 2.67-2.56 (m, 2H), 2.14-2.03 (m, 4H), 2.01 (s, 6H), 1.82 (d, J=15.1 Hz, 1H), 1.62 (s, 3H), 1.51-1.45 (m, 1H), 0.49 (s, 3H), 0.40-0.31 (m, 1H), 0.30-0.20 (m, 1H), 0.18-0.09 (m, 1H), 0.06-0.01 (m, 1H). ESI-MS m/z calc. 692.2781, found 693.5 (M+1)⁺; Retention time: 2.24 minutes. LC method A.

### Example 75: Preparation of Compound I-147

### Step 1: 3-[[[4-[(2R)-2-[(6-Cyclopropylfuro[2,3-b]pyrazin-2-yl)methylamino]-4,4-dimethyl-pentoxy]-6-(2,6-dimethylphenyl)pyrimidin-2-yl]amino]sulfonimidoyl]benzoic acid, isomer A

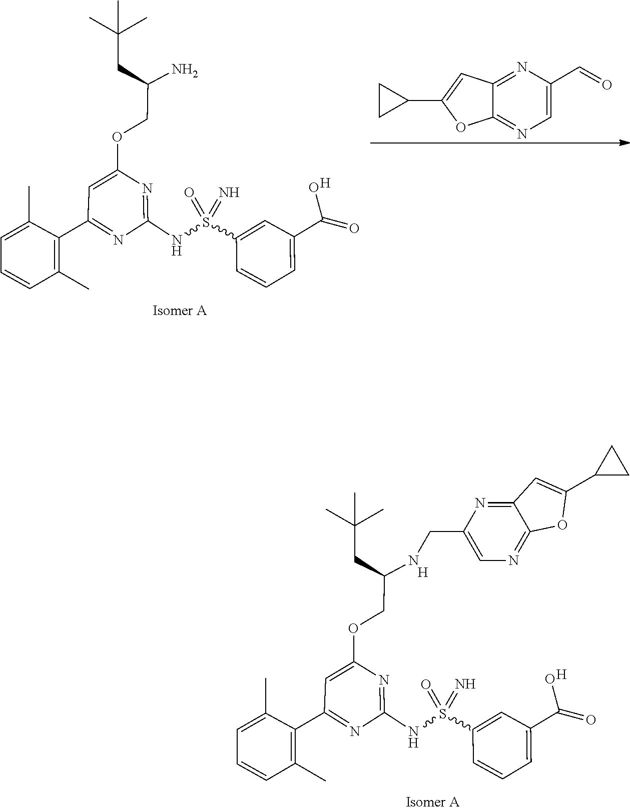

3-[[[4-[(2R)-2-amino-4,4-dimethyl-pentoxy]-6-(2,6-dimethylphenyl)pyrimidin-2-yl]amino]sulfonimidoyl]benzoic acid (hydrochloride salt) (60 mg, 0.1095 mmol) (isomer A) was combined with 6-cyclopropylfuro[2,3-b]pyrazine-2-carbaldehyde (23 mg, 0.1222 mmol) in DCM (0.4 mL) and stirred for 10 minutes at room temperature. sodium triacetoxyborohydride (23 mg, 0.1085 mmol) was added and after another 10 minutes stirring a second portion of sodium triacetoxyborohydride (60 mg, 0.2831 mmol) was added. After 20 minutes an additional portion of 6-cyclopropylfuro[2,3-b]pyrazine-2-carbaldehyde (8 mg, 0.04251 mmol) was added and the reaction was stirred for a further 10 minutes before it was quenched with 0.2 mL 3M HCl (aq.) then diluted with methanol and DMSO until the reaction mixture became homogeneous. The reaction mixture was filtered and purified by preparative HPLC (1-99% ACN in water, HCl modifier, 15 minute run) to give 3-[[[4-[(2R)-2-[(6-cyclopropylfuro[2,3-b]pyrazin-2-yl)methylamino]-4,4-dimethyl-pentoxy]-6-(2,6-dimethylphenyl)pyrimidin-2-yl]amino]sulfonimidoyl]benzoic acid isomer A (hydrochloride salt) (41 mg, 52%) ESI-MS m z calc. 683.289, found 684.7 (M+1)⁺; Retention time: 0.49 minutes. LC method B.

### Step 2: (11R)-12-[(6-Cyclopropylfuro[2,3-b]pyrazin-2-yl)methyl]-6-(2,6-dimethylphenyl)-11-(2,2-dimethylpropyl)-2-imino-2-oxo-9-oxa-2λ⁶-thia-3,5,12,19-tetrazatricyclo[12.3.1.14,8]nonadeca-1(18),4(19),5,7,4,16-hexaen-13-one, isomer A (Compound I-147

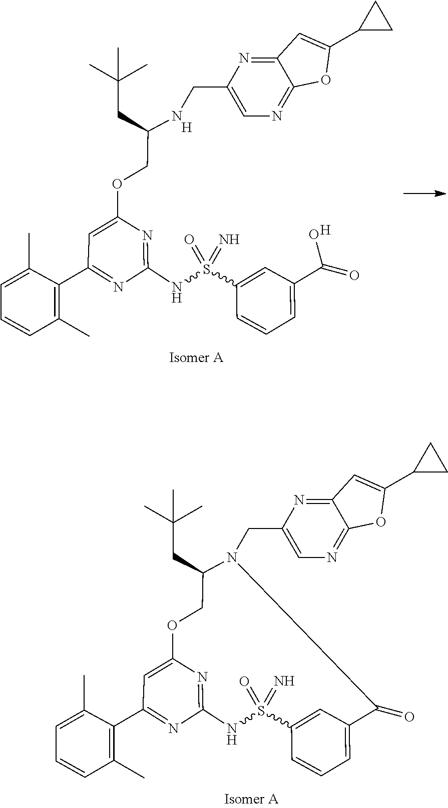

3-[[[4-[(2R)-2-[(6-Cyclopropylfuro[2,3-b]pyrazin-2-yl)methylamino]-4,4-dimethyl-pentoxy]-6-(2,6-dimethylphenyl)pyrimidin-2-yl]amino]sulfonimidoyl]benzoic acid (hydrochloride salt) (41 mg, 0.05692 mmol) (isomer A) was combined with CDMT (12 mg, 0.06835 mmol) in DMF (4 mL) and cooled to 0° C. in an ice bath. N-methylmorpholine (40 μL, 0.3638 mmol) was added, and the reaction was allowed to slowly warm to room temperature as the ice melted with a total of 16 hours stirring. The reaction mixture was then filtered and purified by preparative HPLC(1-99% ACN in water, HCl modifier, 15 minute run) to give (11R)-12-[(6-cyclopropylfuro[2,3-b]pyrazin-2-yl)methyl]-6-(2,6-dimethylphenyl)-11-(2,2-dimethylpropyl)-2-imino-2-oxo-9-oxa-2λ⁶-thia-3,5,12,19-tetrazatricyclo[12.3.1.14,8]nonadeca-1(18),4(19),5,7,14,16-hexaen-13-one isomer A (9.4 mg, 23%) ESI-MS m/z calc. 665.27844, found 666.7 (M+1)⁺; Retention time: 1.66 minutes. LC method A. ¹H NMR (400 MHz, Chloroform-d) δ 8.51 (t, J=1.8 Hz, 1H), 8.35 (s, 1H), 8.29 (d, J=7.9 Hz, 1H), 7.73 (d, J=7.6 Hz, 1H), 7.57 (t, J=7.8 Hz, 1H), 7.02 (t, J=7.5 Hz, 1H), 6.91 (d, J=38.8 Hz, 2H), 6.64 (s, 1H), 6.08 (s, 1H), 5.42 (dd, J=11.2, 4.2 Hz, 1H), 5.24 (d, J=15.0 Hz, 1H), 4.32 (d, J=15.0 Hz, 1H), 4.15 (t, J=11.4 Hz, 1H), 3.95-3.83 (m, 1H), 2.19-2.02 (m, 4H), 1.95-1.71 (m, 4H), 1.53 (d, J=15.0 Hz, 1H), 1.20-1.11 (m, 4H), 0.62 (s, 9H).

### Example 76: Preparation of Compound I-148

### Step 1: 3-[[[4-[(2R)-2-[(6-Cyclopropylfuro[2,3-b]pyrazin-2-yl)methylamino]-4,4-dimethyl-pentoxy]-6-(2,6-dimethylphenyl)pyrimidin-2-yl]amino]sulfonimidoyl]benzoic acid, isomer B

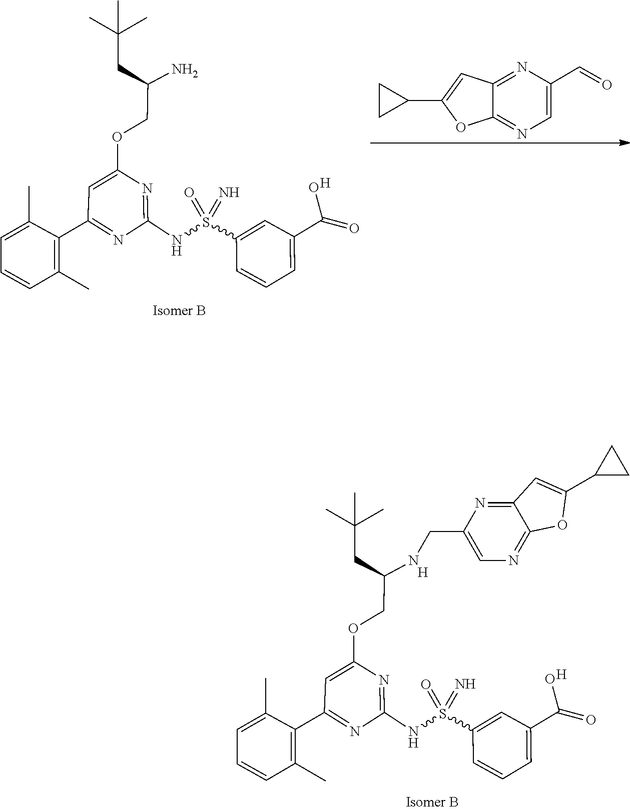

3-[[[4-[(2R)-2-Amino-4,4-dimethyl-pentoxy]-6-(2,6-dimethylphenyl)pyrimidin-2-yl]amino]sulfonimidoyl]benzoic acid (hydrochloride salt) (60 mg, 0.1095 mmol) (isomer B) was combined with 6-cyclopropylfuro[2,3-b]pyrazine-2-carbaldehyde (23 mg, 0.1222 mmol) in DCM (0.4 mL) and stirred for 10 minutes at room temperature. sodium triacetoxyborohydride (23 mg, 0.1085 mmol) was added and after another 10 minutes stirring a second portion of sodium triacetoxyborohydride (60 mg, 0.2831 mmol) was added. After 20 minutes an additional portion of 6-cyclopropylfuro[2,3-b]pyrazine-2-carbaldehyde (8 mg, 0.04251 mmol) was added and the reaction was stirred for a further 10 minutes before it was quenched with 0.2 mL 3M HCl (aq) then diluted with methanol and DMSO until the reaction mixture became homogeneous. The reaction mixture was filtered and purified by preparative HPLC (1-99% ACN in water, HCl modifier, 15 minute run to give 3-[[[4-[(2R)-2-[(6-cyclopropylfuro[2,3-b]pyrazin-2-yl)methylamino]-4,4-dimethyl-pentoxy]-6-(2,6-dimethylphenyl)pyrimidin-2-yl]amino]sulfonimidoyl]benzoic acid isomer B (hydrochloride salt) (30 mg, 38%) ESI-MS m z calc. 683.289, found 684.7 (M+1)⁺; Retention time: 0.5 minutes. LC method B.

### Step 2: (11R)-12-[(6-Cyclopropylfuro[2,3-b]pyrazin-2-yl)methyl]-6-(2,6-dimethylphenyl)-11-(2,2-dimethylpropyl)-2-imino-2-oxo-9-oxa-2λ⁶-thia-3,5,12,19-tetrazatricyclo[12.3.1.14,8]nonadeca-1(18),4(19),5,7,4,16-hexaen-13-one, isomer B (Compound I-148

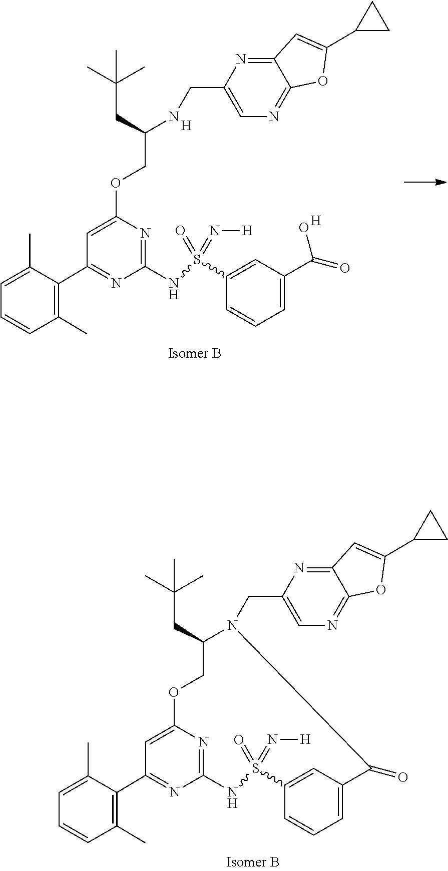

3-[[[4-[(2R)-2-[(6-Cyclopropylfuro[2,3-b]pyrazin-2-yl)methylamino]-4,4-dimethyl-pentoxy]-6-(2,6-dimethylphenyl)pyrimidin-2-yl]amino]sulfonimidoyl]benzoic acid (hydrochloride salt) (30 mg, 0.04165 mmol) (isomer B) was combined with CDMT (9 mg, 0.05126 mmol) in DMF (4 mL) and cooled to 0° C. in an ice bath. N-methylmorpholine (30 μL, 0.2729 mmol) was added, and the reaction was allowed to slowly warm to room temperature as the ice melted with a total of 16 hours stirring. The reaction mixture was then filtered and purified by preparative HPLC(1-99% ACN in water, HCl modifier, 15 minute run), followed by an additional purification by preparative HPLC(1-99% ACN in water HCl modifier, 30 minute run) and finally purification on silica gel eluting with a 0-15% methanol in DCM gradient to give (11R)-12-[(6-cyclopropylfuro[2,3-b]pyrazin-2-yl)methyl]-6-(2,6-dimethylphenyl)-11-(2,2-dimethylpropyl)-2-imino-2-oxo-9-oxa-2λ⁶-thia-3,5,12,19-tetrazatricyclo[12.3.1.14,8]nonadeca-1(18),4(19),5,7,14,16-hexaen-13-one (2.2 mg, 8%) ESI-MS m/z calc. 665.27844, found 666.6 (M+1)⁺; Retention time: 1.61 minutes. LC method A.

### Example 77: Preparation of Compound I-149

### Step 1: N-(3-Bromo-6-chloro-pyrazin-2-yl)-2-methyl-propanamide

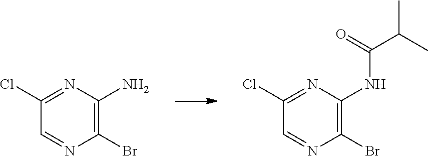

To a solution of 3-bromo-6-chloro-pyrazin-2-amine (4.5 g, 20.509 mmol) in THE (22.5 mL) was added DMAP (251 mg, 2.0545 mmol), DIPEA (5.3424 g, 7.2 mL, 41.336 mmol) and then 2-methylpropanoyl 2-methylpropanoate (4.7032 g, 4.93 mL, 29.730 mmol) at room temperature. The solution was heated to 70° C. for 19 h. The reaction was cooled to rt and diluted with EtOAc (300 mL) and NaHCO₃ (350 mL). The aqueous phase was extracted with EtOAc (3×100 mL), the combined organics were washed with water (100 mL), and the water phase was back extracted with EtOAc. The combined organics were washed with brine (50 mL), and dried over Na₂SO₄, filtered, and concentrated in vacuo to give a yellow residue of solids (6.26 g). The crude residue was purified by flash chromatography (220 g SiO₂, dry loaded, eluted with 0-10% EtOAc hexanes over 30 minutes gradient, 20% EtOAc in hexanes; followed by 100% EtOAc) to obtain the target, N-(3-bromo-6-chloro-pyrazin-2-yl)-2-methyl-propanamide (4.07 g, 71%), as an off white solid. ¹H NMR (500 MHz, Chloroform-d) δ 8.09 (s, 1H), 7.87 (s, 1H), 2.98 (hept, J=6.7 Hz, 1H), 1.30 (d, J=6.9 Hz, 6H). ESI-MS m/z calc. 276.96176, found 278.3 (M+1)⁺; Retention time: 2.78 minutes; LC method D.

### Step 2: 5-Chloro-2-isopropyl-oxazolo[4,5-b]pyrazine

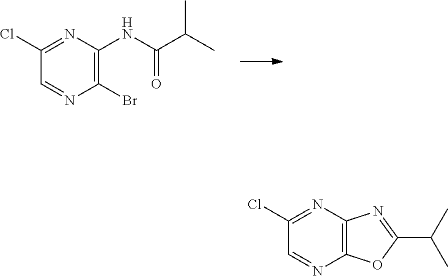

A vial was charged with, Pd(OAc)₂ (38 mg, 0.1693 mmol), Xantphos (161 mg, 0.2782 mmol), Cs₂CO₃ (1.4 g, 4.2969 mmol) and purged with argon. The solids were treated with degassed dioxane (7.5 mL) followed by N-(3-bromo-6-chloro-pyrazin-2-yl)-2-methyl-propanamide (500 mg, 1.7951 mmol) sequentially. The vessel was sealed under argon and the reaction was stirred at 100° C. for 90 min in a microwave reactor. The reaction was diluted with EtOAc, filtered, concentrated in vacuo onto SiO₂ (5 g). The crude residue was purified by flash chromatography (12 g SiO₂, dry loaded, eluted with 0-100% Et₂O in hexanes over a 25 min gradient) to obtain the target, 5-chloro-2-isopropyl-oxazolo[4,5-b]pyrazine (111 mg, 31%), as a colorless liquid. ¹H NMR (500 MHz, Chloroform-d) δ 8.26 (s, 1H), 3.34 (hept, J=6.9 Hz, 1H), 1.50 (d, J=7.0 Hz, 6H). ESI-MS m/z calc. 197.03558, found 198.3 (M+1)⁺; Retention time: 3.57 minutes; LC method D.

### Step 3: 2-Isopropyl-5-vinyl-oxazolo[4,5-b]pyrazine

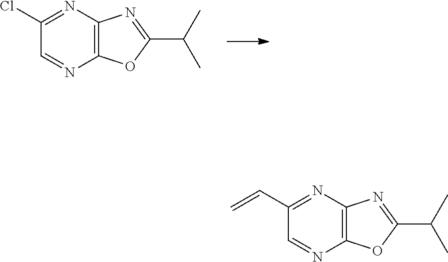

A mixture of 5-chloro-2-isopropyl-oxazolo[4,5-b]pyrazine (96.7 mg, 0.4893 mmol), potassium vinyltrifluoroborate (177 mg, 1.3214 mmol) was treated with degassed EtOH (4.2 mL), Pd(dppf)Cl₂·DCM (32 mg, 0.0392 mmol). Argon was bubbled through the mixture for 5 minutes and TEA (290.40 mg, 0.4 mL, 2.8698 mmol) was added under argon. The vessel was sealed and heated at 50° C. for 2 h and 30 minutes in a microwave reactor. The reaction was concentrated in vacuo and purified by flash chromatography (loaded onto a 4 g SiO₂ cartridge with benzene, and eluted with 0-20% Et₂O in hexanes over a 15 min gradient) to obtain the product, 2-isopropyl-5-vinyl-oxazolo[4,5-b]pyrazine (19 mg, 21%), as a colorless oil. ¹H NMR (500 MHz, Chloroform-d) δ 8.21 (s, 1H), 6.90 (dd, J=17.3, 10.8 Hz, 1H), 6.46 (dd, J=17.3, 1.2 Hz, 1H), 3.32 (hept, J=7.0 Hz, 1H), 1.51 (d, J=7.0 Hz, 6H). ESI-MS m/z calc. 189.09021, found 190.7 (M+1)⁺; Retention time: 3.46 minutes; LC method D.

### Step 4: 2-Isopropyloxazolo[4,5-b]pyrazine-5-carbaldehyde

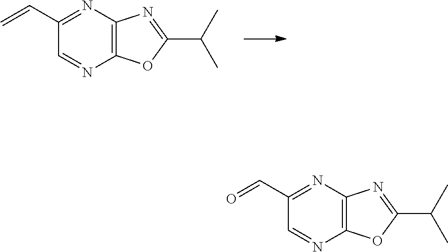

To a solution of 2-isopropyl-5-vinyl-oxazolo[4,5-b]pyrazine (41 mg, 0.217 mmol) in dioxane (0.41 mL) and water (0.14 mL) was added pyridine (36.186 mg, 0.037 mL, 0.4575 mmol), OsO₄ in t-BuOH (0.375 mL of 2.5% w/v, 0.0369 mmol) then NaIO₄ (182 mg, 0.8509 mmol) at room temperature. The reaction was stirred at room temperature 1.5 h. Then additional pyridine (68.460 mg, 0.070 mL, 0.8655 mmol) was added to form a thick paste. The reaction was stirred for an additional 150 minutes at rt. The reaction was diluted with DCM (30 mL) and the organic phase was washed with NaHCO₃ (10 mL). The aqueous phase was extracted with DCM (3×10 mL), washed with brine, dried over Na₂SO₄ filtered and concentrated in vacuo to provide a black crude oil (51 mg) containing the product 2-isopropyloxazolo[4,5-b]pyrazine-5-carbaldehyde. ESI-MS m/z calc. 191.06947, found 192.4 (M+1)⁺; Retention time: 2.16 minutes; LC method D. The crude material from this reaction was combined with another 25 mg crude from a different reaction. The residue was purified by flash chromatography (4 g SiO₂ cartridge, dry loaded, and eluted with 0-30% Et₂O in hexanes over a 15 min gradient) to provide 2-isopropyloxazolo[4,5-b]pyrazine-5-carbaldehyde (26 mg, corrected yield 43%) as a light brown transparent oil. ¹H NMR (400 MHz, Chloroform-d) δ 10.21 (s, 1H), 8.96 (s, 1H), 3.40 (hept, J=7.0 Hz, 1H), 1.55 (d, J=6.7 Hz, 6H). ESI-MS m/z calc. 191.06947, found 192.1 (M+1)⁺; Retention time: 1.57 minutes; LC method H.

### Step 5: 3-[[4-(2,6-Dimethylphenyl)-6-[(2R)-2-[(2-isopropyloxazolo[4,5-b]pyrazin-5-yl)methylamino]-3-(1-methylcyclopropyl)propoxy]pyrimidin-2-yl]sulfamoyl]benzoic acid

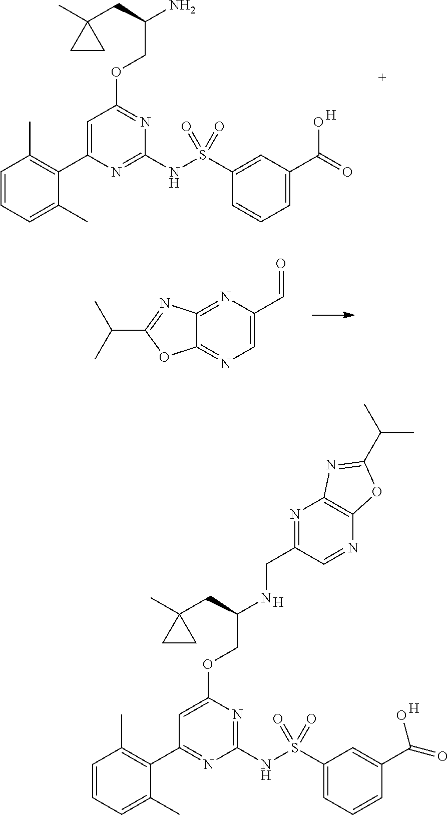

In a 4 mL vial, to a stirred mixture of 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (30 mg, 0.05484 mmol) and 2-isopropyloxazolo[4,5-b]pyrazine-5-carbaldehyde (10.5 mg, 0.05492 mmol) in anhydrous dichloromethane (170 μL) were added glacial acetic acid (10 μL, 0.1758 mmol) and DIPEA (25 μL, 0.1435 mmol), in that order, at 0° C. under nitrogen. After 2 min, sodium triacetoxyborohydride (32 mg, 0.1510 mmol) was added to the yellow solution. The heterogeneous reaction was stirred at that temperature for 15 min. Then the reaction was quenched with acetic acid (0.4 mL), MeOH (0.5 mL) and DMSO (0.5 mL) and purified by preparative reverse-phase HPLC [1-99% acetonitrile in water (containing 5 mM HCl as modifier) over 15 min]. The product fraction was extracted with ethyl acetate (3×15 mL) and the combined organics were washed with brine (15 mL), dried over sodium sulfate, filtered and concentrated under reduced pressure to furnish product as a yellow solid. 3-[[4-(2,6-dimethylphenyl)-6-[(2R)-2-[(2-isopropyloxazolo[4,5-b]pyrazin-5-yl)methylamino]-3-(1-methylcyclopropyl)propoxy]pyrimidin-2-yl]sulfamoyl]benzoic acid (hydrochloride salt) (30 mg, 64%) ESI-MS m/z calc. 685.26825, found 686.2 (M+1)⁺; Retention time: 1.25 minutes. LC method A.

### Step 6: (11R)-6-(2,6-Dimethylphenyl)-12-[(2-isopropyloxazolo[4,5-b]pyrazin-5-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-149

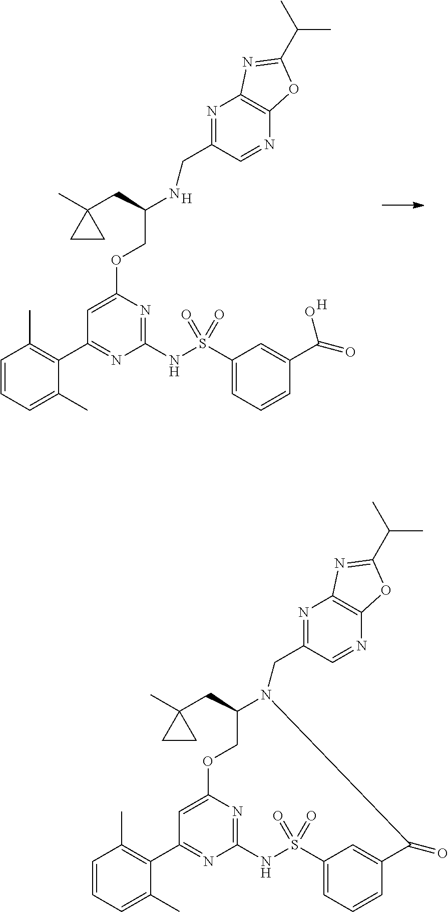

To a stirred solution of 3-[[4-(2,6-dimethylphenyl)-6-[(2R)-2-[(2-isopropyloxazolo[4,5-b]pyrazin-5-yl)methylamino]-3-(1-methylcyclopropyl)propoxy]pyrimidin-2-yl]sulfamoyl]benzoic acid (hydrochloride salt) (30 mg, 0.03531 mmol) in anhydrous DMF (1.5 mL) was added 2-chloro-4,6-dimethoxy-1,3,5-triazine (10 mg, 0.05696 mmol) (CDMT), followed by addition of 4-methylmorpholine (30 μL, 0.2729 mmol) at 0-4° C. (ice-water bath) under nitrogen. The yellow reaction was allowed to stir at that temperature for 5 min, then allowed to stir at room temperature for 2 h. Purified the reaction mixture by reverse-phase HPLC (1-99% acetonitrile in water over 15 min, no modifier). The desired fraction was extracted with ethyl acetate (3×10 mL), dried over sodium sulfate, filtered, and concentrated under reduced pressure to furnish product as a white solid. (11R)-6-(2,6-dimethylphenyl)-12-[(2-isopropyloxazolo[4,5-b]pyrazin-5-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (9.5 mg, 40%) ¹H NMR (400 MHz, MeOD) δ 8.63 (t, J=1.8 Hz, 1H), 8.52 (s, 1H), 8.03 (dt, J=7.8, 1.5 Hz, 1H), 7.74 (dt, J=7.7, 1.4 Hz, 1H), 7.66 (t, J=7.7 Hz, 1H), 7.25 (t, J=7.6 Hz, 1H), 7.13 (d, J=7.7 Hz, 2H), 6.27 (s, 1H), 5.55 (d, J=6.7 Hz, 1H), 5.09 (d, J=15.1 Hz, 1H), 4.58-4.39 (m, 3H), 3.42-3.32 (m, 1H), 2.10 (s, 6H), 1.83 (d, J=15.2 Hz, 1H), 1.59 (dd, J=15.4, 9.4 Hz, 1H), 1.49 (d, J=7.0 Hz, 6H), 0.46 (s, 3H), 0.38 (dt, J=9.6, 4.9 Hz, 1H), 0.24 (dt, J=9.7, 4.9 Hz, 1H), 0.13 (dt, J=9.3, 4.8 Hz, 1H), 0.05 (dt, J=9.7, 4.9 Hz, 1H). ESI-MS m/z calc. 667.2577, found 668.1 (M+1)⁺; Retention time: 1.8 minutes. LC method A.

### Example 78: Preparation of Compound I-157

### Step 1: 3-[[4-(2,6-Dimethylphenyl)-6-[(3S)-3-[(6-isopropylfuro[2,3-b]pyrazin-2-yl)methylamino]-5,5-dimethyl-hexyl]-5-methoxy-pyrimidin-2-yl]sulfamoyl]benzoic acid

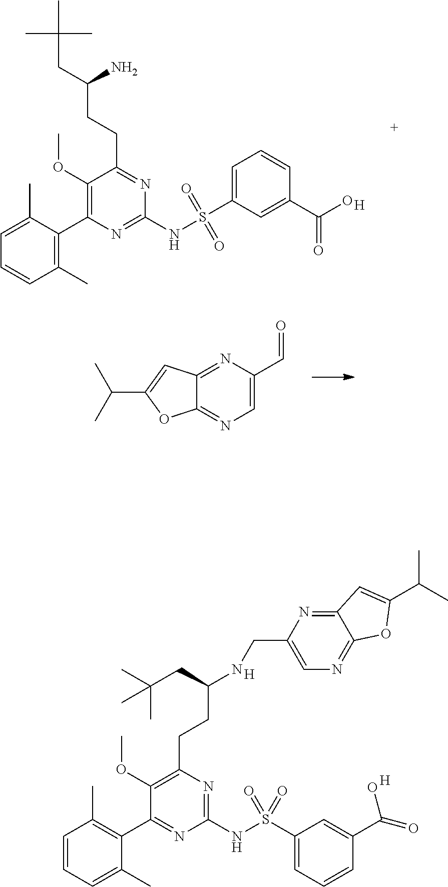

A 4 mL vial was charged under nitrogen with 3-[[4-[(3S)-3-amino-5,5-dimethyl-hexyl]-6-(2,6-dimethylphenyl)-5-methoxy-pyrimidin-2-yl]sulfamoyl]benzoic acid (90 mg, 0.1665 mmol), 6-isopropylfuro[2,3-b]pyrazine-2-carbaldehyde (34.4 mg, 0.1809 mmol), anhydrous DCM (0.81 mL), and acetic acid (9.6 μL, 0.1688 mmol). The mixture was cooled down in an ice bath. DIEA (57.6 μL, 0.3307 mmol) was added, followed by sodium triacetoxyborohydride (185.6 mg, 0.8757 mmol), and the reaction was vigorously stirred at 0° C. for 1 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-(2,6-dimethylphenyl)-6-[(3S)-3-[(6-isopropylfuro[2,3-b]pyrazin-2-yl)methylamino]-5,5-dimethyl-hexyl]-5-methoxy-pyrimidin-2-yl]sulfamoyl]benzoic acid (hydrochloride salt) (77.8 mg, 61%) as a yellow solid. ESI-MS m z calc. 714.31995, found 715.5 (M+1)⁺; Retention time: 1.75 minutes. LC method A.

### Step 2: (11S)-6-(2,6-Dimethylphenyl)-11-(2,2-dimethylpropyl)-12-[(6-isopropylfuro[2,3-b]pyrazin-2-yl)methyl]-7-methoxy-2,2-dioxo-2λ⁶-thia-3,5,12,19-tetrazatricyclo[12.3.1.14,8]nonadeca-1(18),4(19),5,7,14,16-hexaen-13-one (Compound I-157

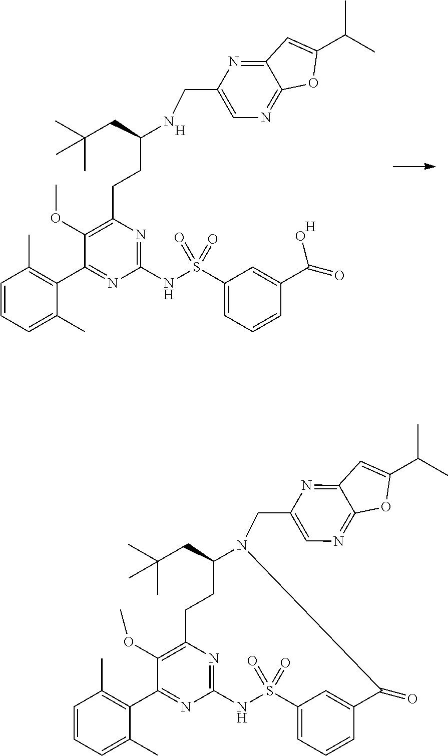

3-[[4-(2,6-Dimethylphenyl)-6-[(3S)-3-[(6-isopropylfuro[2,3-b]pyrazin-2-yl)methylamino]-5,5-dimethyl-hexyl]-5-methoxy-pyrimidin-2-yl]sulfamoyl]benzoic acid (hydrochloride salt) (77.8 mg, 0.1015 mmol) was combined with CDMT (28.3 mg, 0.1612 mmol) in DMF (9.2 mL) and cooled to 0° C. N-methylmorpholine (33.5 μL, 0.3047 mmol) was added by syringe and the reaction was stirred at 0° C. for 30 minutes. The ice bath was then removed and stirring was continued for an additional 3 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO and MeOH, filtered, and purified by reverse phase HPLC (1-99% acetonitrile/5 mM aqueous HCl over 25 min) to give (11S)-6-(2,6-dimethylphenyl)-11-(2,2-dimethylpropyl)-12-[(6-isopropylfuro[2,3-b]pyrazin-2-yl)methyl]-7-methoxy-2,2-dioxo-2λ⁶-thia-3,5,12,19-tetrazatricyclo[12.3.1.14,8]nonadeca-1(18),4(19),5,7,14,16-hexaen-13-one (43.5 mg, 62%) as a white-yellow solid. ¹H NMR (400 MHz, CDCl₃) δ 8.83 (t, J=1.8 Hz, 1H), 8.42 (s, 1H), 8.07 (dt, J=8.0, 1.5 Hz, 1H), 7.89 (dt, J=7.7, 1.4 Hz, 1H), 7.70-7.66 (m, 1H), 7.61 (t, J=7.8 Hz, 1H), 7.22 (t, J=7.6 Hz, 1H), 7.11 (d, J=7.5 Hz, 1H), 7.05 (d, J=7.6 Hz, 1H), 6.62 (s, 1H), 5.26 (d, J=15.1 Hz, 1H), 4.40 (d, J=15.2 Hz, 1H), 4.15-3.98 (m, 1H), 3.34-3.22 (m, 4H), 3.17 (hept, J=1.1 Hz, 1H), 3.03-2.90 (m, 1H), 2.84 (dt, J=17.8, 4.2 Hz, 1H), 2.21-2.12 (m, 1H), 2.10 (s, 3H), 1.89 (s, 3H), 1.66 (dd, J=15.0, 9.4 Hz, 1H), 1.40 (d, J=6.9 Hz, 6H), 1.34-1.23 (m, 1H), 0.54 (s, 9H). ESI-MS m/z calc. 696.3094, found 697.5 (M+1)⁺; Retention time: 2.37 minutes. LC method A.

### Example 79: Preparation of Compound I-159

### Step 1: N-(3,5-Dichloropyrazin-2-yl)-2-methyl-propanamide

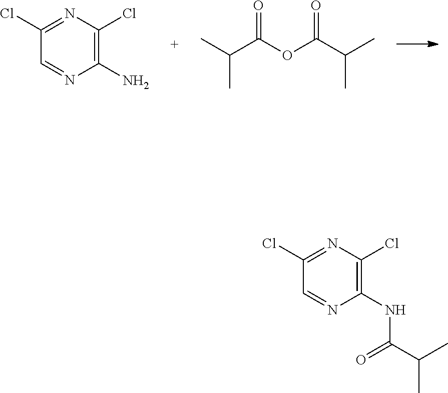

To a solution of 3,5-dichloropyrazin-2-amine (25 g, 144.82 mmol) was added 2-methylpropanoyl 2-methylpropanoate (68.688 g, 72 mL, 434.20 mmol) at room temperature. The reaction was heated at 130° C. for 18 h. The reaction was diluted EtOAc (1.5 L), washed with a mixture of H₂O (300 mL) and sat. Na₂CO₃ (300 mL) three times. A precipitate formed that was dissolved in H₂O (800 mL), the combined aqueous phase was extracted with EtOAc (2×500 mL), washed with brine (500 mL). The combined organic layers were washed dried over Na₂SO₄, filtered and concentrated in vacuo to give a crude residue as a tan, orange paste. Then hexanes (1 L) were added, and the mixture was briefly heated, cooled to rt for 30 min, filtered, and the solids were rinsed thoroughly with hexanes (1 L). The filter cake was pressed between 2 filter papers to aid removal of residual solvent, and further dried in vacuo to give, N-(3,5-dichloropyrazin-2-yl)-2-methyl-propanamide (27.25 g, 80%), as a light orange fine solid needles. ¹H NMR (500 MHz, Chloroform-d) δ 8.35 (s, 1H), 7.78 (s, 1H), 2.85 (hept, J=6.8 Hz, 1H), 1.30 (d, J=6.8 Hz, 6H). ESI-MS m/z calc. 233.01227, found 234.3 (M+1)⁺; Retention time: 3.1 minutes; LC method D.

### Step 2: 6-Chloro-2-isopropyl-oxazolo[4,5-b]pyrazine

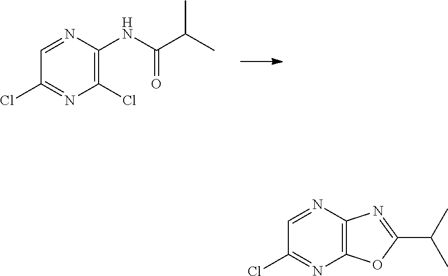

A vial charged with N-(3,5-dichloropyrazin-2-yl)-2-methyl-propanamide (1 g, 4.2720 mmol), cesium carbonate (2.09 g, 6.4146 mmol) was purged with argon. The solids were treated with degassed dioxane (16 mL). The mixture was further degassed for 15 min with argon. Then Pd(OAc)₂ (96 mg, 0.4276 mmol) and Xantphos (495 mg, 0.8555 mmol) were added, and the mixture was sparged for an additional 15 minutes, and then sparged with argon for an additional 30 minutes. The reaction was stirred at 100° C. for 3 h in an oil bath. Cesium carbonate (0.5 g, 1.5346 mmol) was added to the reaction at 100° C. and heating was continued for 30 min. The reaction was cooled to rt, diluted with EtOAc, filtered, and concentrated in vacuo. The crude residue (1.27 g) was purified by flash chromatography (12 g SiO₂, dry loaded on SiO₂, eluted with 0-30% Et₂O in hexanes over a 15 minute gradient) to obtain 6-chloro-2-isopropyl-oxazolo[4,5-b]pyrazine (72.5 mg, 9%), as a yellow oil which solidified to a pale wax upon drying. ¹H NMR (500 MHz, Chloroform-d) δ 8.54 (s, 1H), 3.33 (hept, J=7.0 Hz, 1H), 1.51 (d, J=7.1 Hz, 6H). ESI-MS m/z calc. 197.03558, found 198.4 (M+1)⁺; Retention time: 3.53 minutes; LC method D.

### Step 3: 2-Isopropyl-6-vinyl-oxazolo[4,5-b]pyrazine

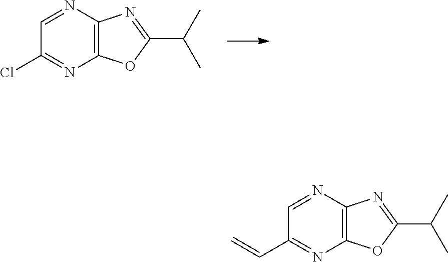

A mixture of 6-chloro-2-isopropyl-oxazolo4,5-pyrazine (107 mg, 0.5144 mmol), potassium vinyltrifluoroborate (176 mg, 1.3139 mmol), Pd(dppf)Cl₂·DCM (41.5 mg, 0.0508 mmol) was treated with degassed EtOH (2.7 mL) and TEA (312.18 mg, 0.43 mL, 3.0851 mmol) under argon. The reaction was heated at 50° C. for 2 h and then for 30 min in a microwave reactor. The reaction was concentrated in vacuo to give a crude residue containing the target, 2-isopropyl-6-vinyl-oxazolo[4,5-b]pyrazine (32.0 mg, 33%). ESI-MS m/z calc. 189.09021, found 190.3 (M+1)⁺; Retention time: 3.54 minutes; LC method D.

### Step 4: 2-Isopropyloxazolo[4,5-b]pyrazine-6-carbaldehyde

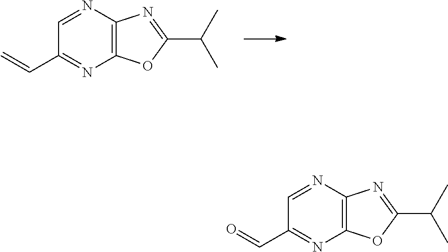

To a solution of 2-isopropyl-6-vinyl-oxazolo[4,5-b]pyrazine (39.8 mg, 0.210 mmol) in dioxane (0.4 mL) and water (0.136 mL) was added pyridine (35.208 mg, 0.036 mL, 0.4451 mmol), OsO₄ in tBuOH (0.365 mL of 2.5% w/v, 0.0359 mmol) then NaIO₄ (179 mg, 0.8369 mmol) at room temperature. The reaction was stirred at room temperature for 2 h and 50 min. The reaction was diluted with DCM (30 mL) washed with NaHCO₃ (10 mL), extracted with DCM (3×10 mL), washed with brine (2×25 mL), dried over Na₂SO₄ filtered and concentrated in vacuo to provide a black oil. The residue was purified by flash chromatography (4 g SiO₂ cartridge, dry loaded, and eluted with 0-30% Et₂O in hexanes over a 15 min gradient) to provide 2-isopropyloxazolo[4,5-b]pyrazine-6-carbaldehyde (9.4 mg, 23%) as a transparent oil. ¹H NMR (500 MHz, Chloroform-d) δ 10.15 (s, 1H), 9.20 (s, 1H), 3.41 (hept, J=6.9, Hz, 1H), 1.55 (d, J=7.1 Hz, 6H). ESI-MS m/z calc. 191.06947, found 192.1 (M+1)⁺; Retention time: 1.41 minutes; LC method H.

### Step 5: 3-[[4-(2,6-Dimethylphenyl)-6-[(2R)-2-[(2-isopropyloxazolo[4,5-b]pyrazin-6-yl)methylamino]-3-(1-methylcyclopropyl)propoxy]pyrimidin-2-yl]sulfamoyl]benzoic acid

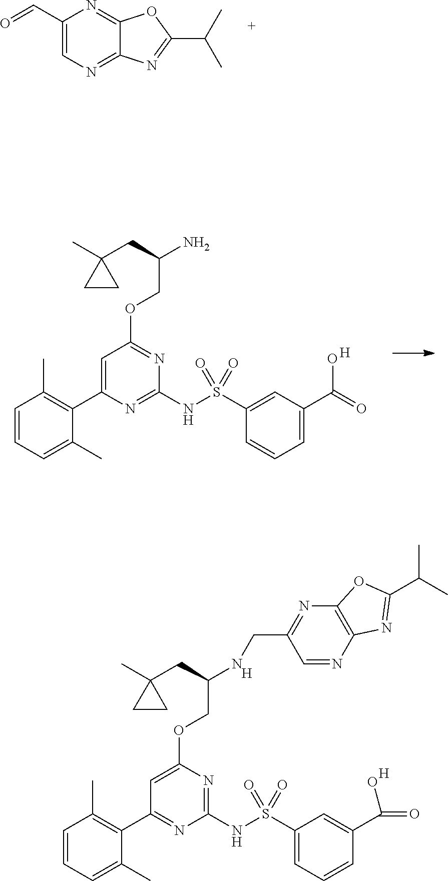

In a 4 mL vial, to a stirred mixture of 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (35 mg, 0.06398 mmol) and 2-isopropyloxazolo[4,5-b]pyrazine-6-carbaldehyde (9.4 mg, 0.04917 mmol) in anhydrous dichloromethane (100 μL) were added glacial acetic acid (10 μL, 0.1758 mmol) and DIPEA (45 μL, 0.2584 mmol), in that order, at 0° C. under nitrogen. After 2 min, sodium triacetoxyborohydride (40 mg, 0.1887 mmol) was added to the yellow solution. The heterogeneous reaction was stirred at that temperature for 15 min. Then the reaction was quenched with MeOH (0.5 mL) and DMSO (0.5 mL) and purified by preparative reverse-phase HPLC [1-99% acetonitrile in water (containing 5 mM HCl) over 15 min]. The desired fraction was extracted with ethyl acetate (3×10 mL) and the combined organics were washed with brine (15 mL), dried over sodium sulfate, filtered, and concentrated under reduced pressure to furnish product as a white solid. 3-[[4-(2,6-dimethylphenyl)-6-[(2R)-2-[(2-isopropyloxazolo[4,5-b]pyrazin-6-yl)methylamino]-3-(1-methylcyclopropyl)propoxy]pyrimidin-2-yl]sulfamoyl]benzoic acid (hydrochloride salt) (25 mg, 70%) ESI-MS m/z calc. 685.26825, found 686.1 (M+1)⁺; Retention time: 1.23 minutes. LC method A.

### Step 6: (11R)-6-(2,6-Dimethylphenyl)-12-[(2-isopropyloxazolo[4,5-b]pyrazin-6-yl)methyl]-11-[(I-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-159

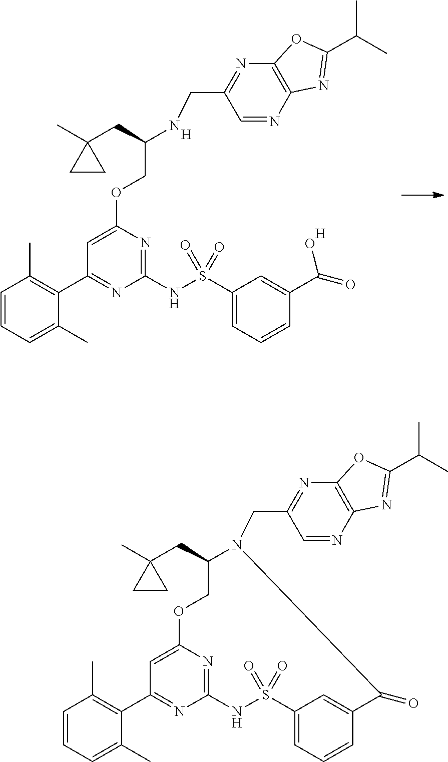

To a stirred solution of 3-[[4-(2,6-dimethylphenyl)-6-[(2R)-2-[(2-isopropyloxazolo[4,5-b]pyrazin-6-yl)methylamino]-3-(1-methylcyclopropyl)propoxy]pyrimidin-2-yl]sulfamoyl]benzoic acid (hydrochloride salt) (25 mg, 0.03461 mmol) in anhydrous DMF (1.3 mL) was added 2-chloro-4,6-dimethoxy-1,3,5-triazine (10 mg, 0.05696 mmol) (CDMT), followed by addition of 4-methylmorpholine (30 μL, 0.2729 mmol) at 0-4° C. (ice-water bath) under nitrogen. The yellow reaction was allowed to stir at that temperature for 5 min, then allowed to stir at room temperature for 2 h. Purified the reaction mixture by preparative reverse-phase HPLC (1-99% acetonitrile in water over 20 min, no modifier) and the desired fraction was concentrated and dried under reduced pressure to furnish product as a yellow solid. (11R)-6-(2,6-dimethylphenyl)-12-[(2-isopropyloxazolo[4,5-b]pyrazin-6-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (6 mg, 26%) ¹H NMR (400 MHz, MeOD) δ 8.71 (s, 1H), 8.69 (s, 1H), 8.03 (d, J=7.7 Hz, 1H), 7.78-7.71 (m, 1H), 7.67 (t, J=7.5 Hz, 1H), 7.26 (t, J=7.6 Hz, 1H), 7.14 (d, J=7.6 Hz, 2H), 6.29 (s, 1H), 5.58 (dd, J=10.6, 3.9 Hz, 1H), 5.11 (d, J=15.6 Hz, 1H), 4.53 (d, J=15.6 Hz, 1H), 4.50-4.41 (m, 1H), 4.37 (t, J=11.2 Hz, 1H), 3.44-3.32 (m, 1H), 2.10 (s, 6H), 1.83 (d, J=15.2 Hz, 1H), 1.55 (dd, J=15.3, 9.7 Hz, 1H), 1.49 (d, J=7.0 Hz, 6H), 0.48 (s, 3H), 0.39 (dt, J=9.4, 4.8 Hz, 1H), 0.25 (dt, J=9.5, 4.9 Hz, 1H), 0.14 (dt, J=9.3, 4.7 Hz, 1H), 0.05 (dt, J=9.7, 4.9 Hz, 1H). ESI-MS m/z calc. 667.2577, found 668.7 (M+1)⁺; Retention time: 1.81 minutes. LC method A.

### Example 80: Preparation of Compound I-160

### Step 1: (11R)-6-(2,6-Dimethylphenyl)-11-(2,2-dimethylpropyl)-12-[[5-(2-hydroxyethyl)-6-isopropyl-pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-160

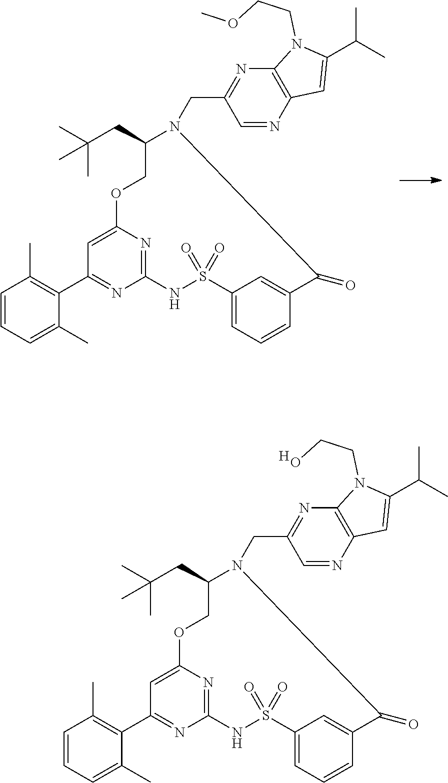

To a stirred solution of (11R)-6-(2,6-dimethylphenyl)-11-(2,2-dimethylpropyl)-12-[[6-isopropyl-5-(2-methoxyethyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (20 mg, 0.02728 mmol) in anhydrous dichloromethane (1 mL) was added tribromoborane (250 μL of 1.0 M, 0.2500 mmol) (in dichloromethane) dropwise at −78° C. under nitrogen. After the addition the reaction was stirred at that temperature for 30 min, then the reaction was allowed to warm to ambient temperature overnight (total 13 h). The volatiles were removed, and the residue was purified by preparative reverse-phase HPLC (1-99% acetonitrile in water over 15 min, 5 mM HCl as modifier) to furnish product as a yellow solid. (11R)-6-(2,6-Dimethylphenyl)-11-(2,2-dimethylpropyl)-12-[[5-(2-hydroxyethyl)-6-isopropyl-pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (11 mg, 56%) ¹H NMR (400 MHz, MeOD) δ 8.88 (t, J=1.8 Hz, 1H), 8.52 (s, 1H), 8.05 (dt, J=7.6, 1.6 Hz, 1H), 7.75 (dt, J=7.6, 1.5 Hz, 1H), 7.70 (t, J=7.7 Hz, 1H), 7.28 (t, J=7.6 Hz, 1H), 7.15 (d, J=7.6 Hz, 2H), 6.57 (s, 1H), 6.32 (s, 1H), 5.72 (dd, J=10.6, 3.9 Hz, 1H), 5.23 (d, J=16.0 Hz, 1H), 4.77 (d, J=16.1 Hz, 1H), 4.70-4.55 (m, 2H), 4.40-4.28 (m, 1H), 4.28-4.19 (m, 1H), 4.04-3.97 (m, 1H), 3.97-3.89 (m, 1H), 3.49 (hept, J=6.8 Hz, 1H), 2.10 (s, 6H), 1.89 (dd, J=15.3, 8.7 Hz, 1H), 1.64 (dd, J=15.3, 1.5 Hz, 1H), 1.41 (d, J=6.5 Hz, 3H), 1.40 (d, J=6.5 Hz, 3H), 0.70 (s, 9H). ESI-MS m/z calc. 711.3203, found 712.8 (M+1)⁺; Retention time: 1.63 minutes. LC method A.

### Example 81: Preparation of Compound I-161

### Step 1: Methyl 7-chloro-6-isopropyl-furo[2,3-b]pyrazine-2-carboxylate

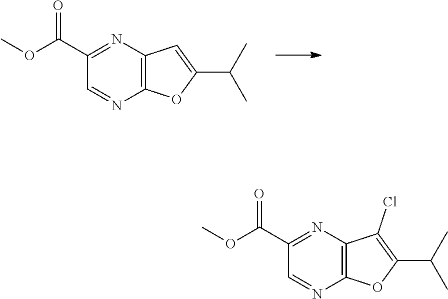 [SMILES: COC(=O)c1cnc2oc(C(C)C)cc2n1 ; COC(=O)c1cnc2oc(C(C)C)c(Cl)c2n1]

To a solution of methyl 6-isopropylfuro[2,3-b]pyrazine-2-carboxylate (880.5 mg, 3.7983 mmol) in DMF (18 mL) at 0° C. was added NCS (1.52 g, 11.383 mmol). The reaction mixture was stirred at 60° C. for 3 h. The reaction mixture was quenched with NaHCO₃ (25 mL), diluted with EtOAc (80 mL), washed with sodium bisulfate solution (20 mL), water (3×20 mL), dried over Na₂SO₄, filtered, concentrated in vacuo. The residue was purified by silica gel chromatography using 0-40% ethyl acetate in hexanes to furnish methyl 3,7-dichloro-6-isopropyl-furo[2,3-b]pyrazine-2-carboxylate (615.2 mg, 54%) as off white solid. ¹H NMR (500 MHz, Chloroform-d) δ 9.06 (s, 1H), 4.07 (s, 3H), 3.48 (hept, J=6.9 Hz, 1H), 1.44 (d, J=7.1 Hz, 6H). ESI-MS m/z calc. 254.04582, found 255.1 (M+1)⁺; Retention time: 2.85 minutes; LC method E.

### Step 2: 7-Chloro-6-isopropyl-furo[2,3-b]pyrazine-2-carbaldehyde

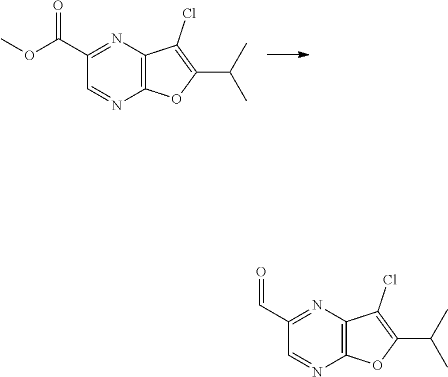 [SMILES: COC(=O)c1cnc2oc(C(C)C)c(Cl)c2n1 ; CC(C)c1oc2ncc(C=O)nc2c1Cl]

To a stirred solution of methyl 7-chloro-6-isopropyl-furo[2,3-b]pyrazine-2-carboxylate (538.3 mg, 2.0292 mmol) in DCM (15 mL) was added DIBAL in DCM (3.3 mL of 1 M, 3.3000 mmol) at −78° C. The reaction mixture was stirred at same temperature for 1 h 50 min. The reaction mixture was quenched with MeOH (10 mL)/water (10 mL) at −78° C. and temperature raised to rt. DCM (100 mL) was added and mass was filtered. The cake was rinsed with DCM (100 mL). The filtrate was separated, washed with brine (2×50 ml), dried over sodium sulfate, filtered and concentrated in vacuo. The residue was purified by silica gel chromatography by using 0-25% ethyl acetate in hexanes to afford 7-chloro-6-isopropyl-furo[2,3-b]pyrazine-2-carbaldehyde (379.5 mg, 81%) as a white solid. ¹H NMR (500 MHz, DMSO-d6) δ 10.17 (s, 1H), 8.93 (s, 1H), 3.47 (hept, J=7.0 Hz, 1H), 1.39 (d, J=7.0 Hz, 6H). ESI-MS m/z calc. 224.03525, found 225.1 (M+1)⁺; Retention time: 2.45 minutes; LC method H.

### Step 3: 3-[[4-[(2R)-2-[(7-Chloro-6-isopropyl-furo[2,3-b]pyrazin-2-yl)methylamino]-3-(1-methylcyclopropyl)propoxy]-6-(2,6-dimethylphenyl)pyrimidin-2-yl]sulfamoyl]benzoic acid

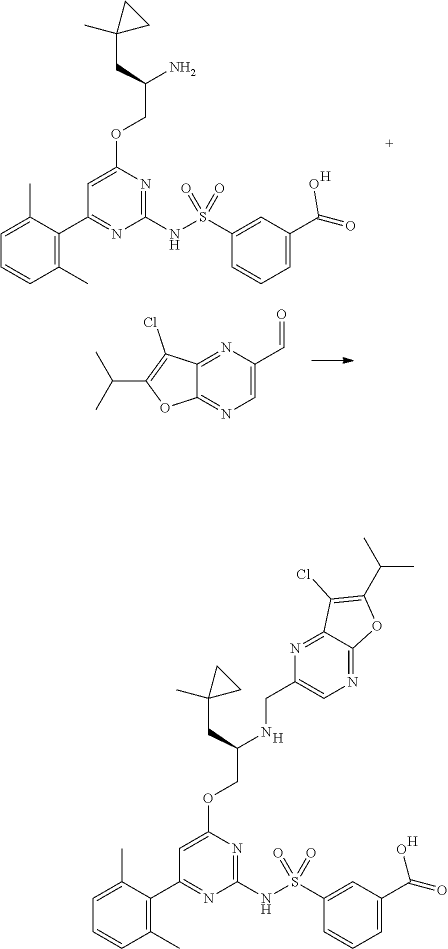

3-[[4-[(2R)-2-Amino-3-(1-methylcyclopropyl)propoxy]-6-(2,6-dimethylphenyl)pyrimidin-2-yl]sulfamoyl]benzoic acid (hydrochloride salt) (60 mg, 0.1097 mmol) was combined with 7-chloro-6-isopropyl-furo[2,3-b]pyrazine-2-carbaldehyde (27 mg, 0.1202 mmol) in DCM (0.4 mL) and stirred for 20 minutes at room temperature. Sodium triacetoxyborohydride (23 mg, 0.1085 mmol) was added and the reaction was stirred for 15 minutes at room temperature, before an additional portion of Sodium triacetoxyborohydride (70 mg, 0.3303 mmol) was added. After 2 hours at room temperature the reaction mixture was quenched with 3M HCl then diluted with methanol and DMSO until the reaction mixture became homogeneous. It was then filtered and purified by preparative HPLC (1-99% ACN in water, HCl modifier, 15 minute run) to give 3-[[4-[(2R)-2-[(7-chloro-6-isopropyl-furo[2,3-b]pyrazin-2-yl)methylamino]-3-(1-methylcyclopropyl)propoxy]-6-(2,6-dimethylphenyl)pyrimidin-2-yl]sulfamoyl]benzoic acid (hydrochloride salt) (45 mg, 54%). ESI-MS m/z calc. 718.234, found 719.7 (M+1)⁺; Retention time: 0.61 minutes; LC method B.

### Step 4: (11R)-12-[(7-Chloro-6-isopropyl-furo[2,3-b]pyrazin-2-yl)methyl]-6-(2,6-dimethylphenyl)-11-[(I-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-161

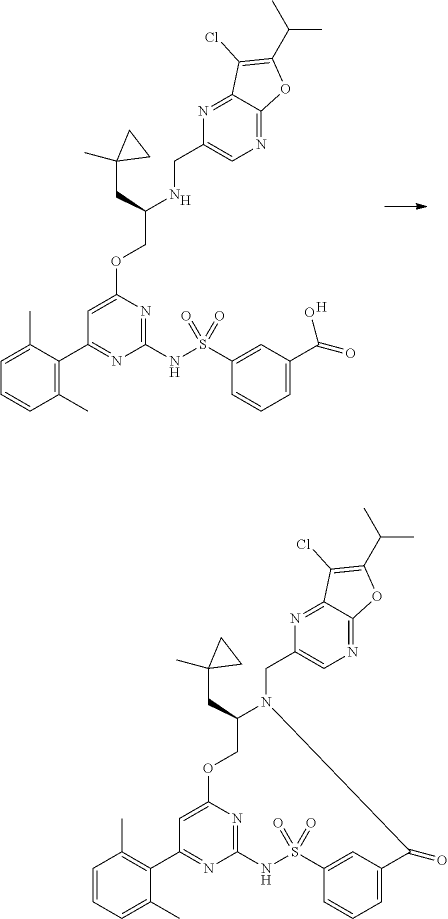

3-[[4-[(2R)-2-[(7-Chloro-6-isopropyl-furo[2,3-b]pyrazin-2-yl)methylamino]-3-(1-methylcyclopropyl)propoxy]-6-(2,6-dimethylphenyl)pyrimidin-2-yl]sulfamoyl]benzoic acid (hydrochloride salt) (45 mg, 0.05955 mmol) was combined with CDMT (13 mg, 0.07404 mmol) in DMF (4 mL) and cooled to 0° C. in an ice bath. N-methylmorpholine (50 μL, 0.4548 mmol) was added and after one hour at 0° C. the reaction was warmed to room temperature and stirred for 18 additional hours. The reaction mixture was then filtered and purified by preparative HPLC (1-99% ACN in water, HCl modifier, 15 minute run) to give (11R)-12-[(7-chloro-6-isopropyl-furo[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (23.4 mg, 54%). ESI-MS m/z calc. 700.22345, found 701.6 (M+1)⁺; Retention time: 2.13 minutes; LC method A. ¹H NMR (400 MHz, Chloroform-d) δ 8.73 (t, J=1.8 Hz, 1H), 8.52 (s, 1H), 8.07 (dt, J=8.1, 1.4 Hz, 1H), 7.86 (dt, J=7.7, 1.4 Hz, 1H), 7.63 (t, J=7.8 Hz, 1H), 7.22 (t, J=7.6 Hz, 1H), 7.07 (d, J=7.6 Hz, 2H), 6.27 (s, 1H), 5.40 (dd, J=10.7, 3.8 Hz, 1H), 5.30 (d, J=14.9 Hz, 1H), 4.49-4.33 (m, 2H), 4.23 (d, J=14.8 Hz, 1H), 3.46 (dq, J=13.9, 7.0 Hz, 1H), 2.03 (s, 6H), 1.83 (d, J=15.2 Hz, 1H), 1.49 (dd, J=15.2, 9.6 Hz, 1H), 1.42 (d, J=7.0, 1.1 Hz, 6H), 0.46 (s, 3H), 0.34 (dq, J=9.8, 4.9 Hz, 1H), 0.24 (dt, J=9.7, 5.0 Hz, 1H), 0.13 (dt, J=9.4, 4.8 Hz, 1H), 0.02 (s, 1H). Sulfonamide NH not visible.

### Example 82: Preparation of Compound I-165 and Compound I-166

### Step 1: 3-[[4-[2-[(6-Cyclopropylfuro[2,3-b]pyrazin-2-yl)methylamino]-3-(3,3-dimethylcyclobutyl)propoxy]-6-(2,6-dimethylphenyl)pyrimidin-2-yl]sulfamoyl]benzoic acid

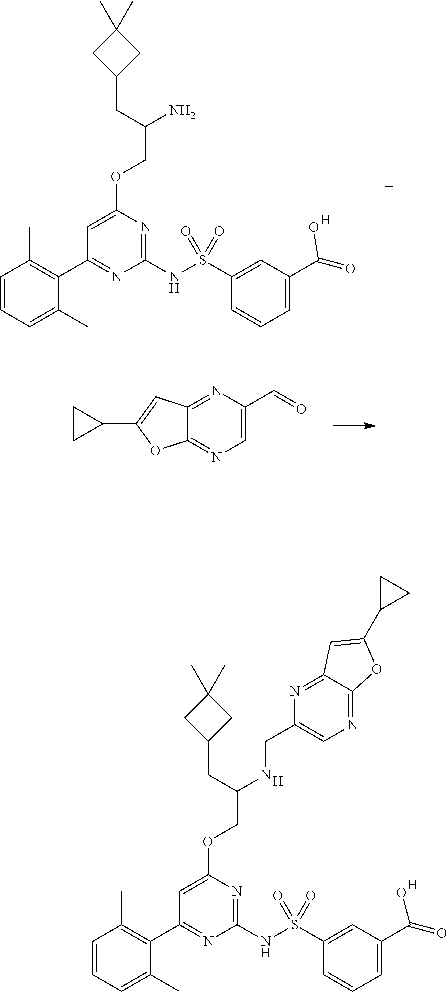

3-[[4-[2-Amino-3-(3,3-dimethylcyclobutyl)propoxy]-6-(2,6-dimethylphenyl)pyrimidin-2-yl]sulfamoyl]benzoic acid (hydrochloride salt) (100 mg, 0.1739 mmol) was combined with 6-cyclopropylfuro[2,3-b]pyrazine-2-carbaldehyde (36.5 mg, 0.1940 mmol) in DCM (0.5 mL) and stirred for 15 minutes at room temperature. sodium triacetoxyborohydride (37 mg, 0.1746 mmol) was added and the reaction was stirred for 30 minutes before additional sodium triacetoxyborohydride (90 mg, 0.4246 mmol) was added. The reaction was stirred for 2.5 hours at room temperature, then was quenched with 0.3 mL 3 M HCl and diluted with methanol and DMSO until it became homogeneous. The reaction mixture was then filtered and purified by preparative HPLC (1-99% ACN in water, HCl modifier, 15 minute run) to give 3-[[4-[2-[(6-cyclopropylfuro[2,3-b]pyrazin-2-yl)methylamino]-3-(3,3-dimethylcyclobutyl)propoxy]-6-(2,6-dimethylphenyl)pyrimidin-2-yl]sulfamoyl]benzoic acid (hydrochloride salt) (95 mg, 73%). ESI-MS m/z calc. 710.28864, found 711.8 (M+1)⁺; Retention time: 0.6 minutes; LC method B.

### Step 2: 12-[(6-Cyclopropylfuro[2,3-b]pyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one

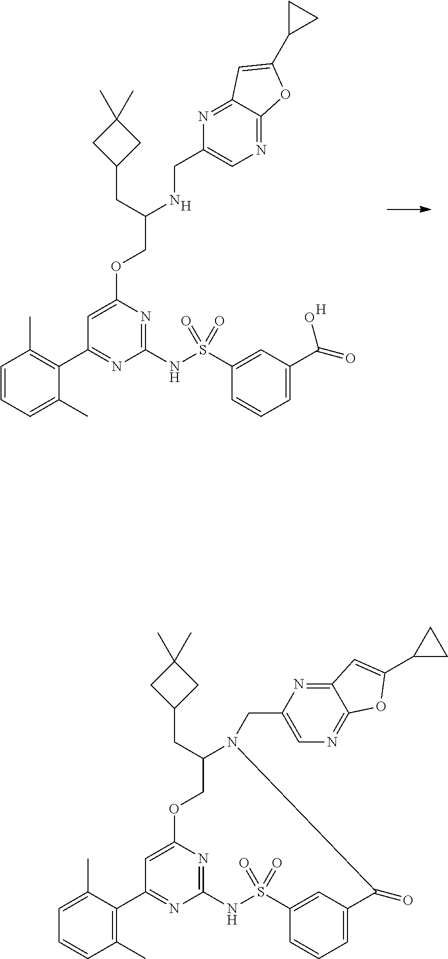

3-[[4-[2-[(6-Cyclopropylfuro[2,3-b]pyrazin-2-yl)methylamino]-3-(3,3-dimethylcyclobutyl)propoxy]-6-(2,6-dimethylphenyl)pyrimidin-2-yl]sulfamoyl]benzoic acid (hydrochloride salt) (95 mg, 0.1271 mmol) was combined with CDMT (27 mg, 0.1538 mmol) in DMF (8 mL) and cooled to 0° C. in an ice bath. N-methylmorpholine (80 μL, 0.7277 mmol) was added by syringe. After one hour at 0° C. the ice bath was removed and stirring was continued at room temperature for 24 hours. The reaction mixture was then concentrated by rotary evaporation (bath temp 50° C.) and the resulting residue was diluted with 15 mL ethyl acetate and 15 mL 1 M HCl and the layers were separated. The aqueous was extracted an additional 2×15 mL ethyl acetate and the combined organics were washed with brine, dried over sodium sulfate and concentrated. The resulting crude was purified by chromatography on silica gel eluting with a 0-10% methanol in DCM gradient to give 12-[(6-cyclopropylfuro[2,3-b]pyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (66.3 mg, 75%). ESI-MS m/z calc. 692.2781, found 693.8 (M+1)⁺; Retention time: 0.82 minutes; LC method B.

### Step 3: 12-[(6-Cyclopropylfuro[2,3-b]pyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, isomer 1, Compound I-165, and 12-[(6-cyclopropylfuro[2,3-b]pyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, isomer 2, Compound I-166

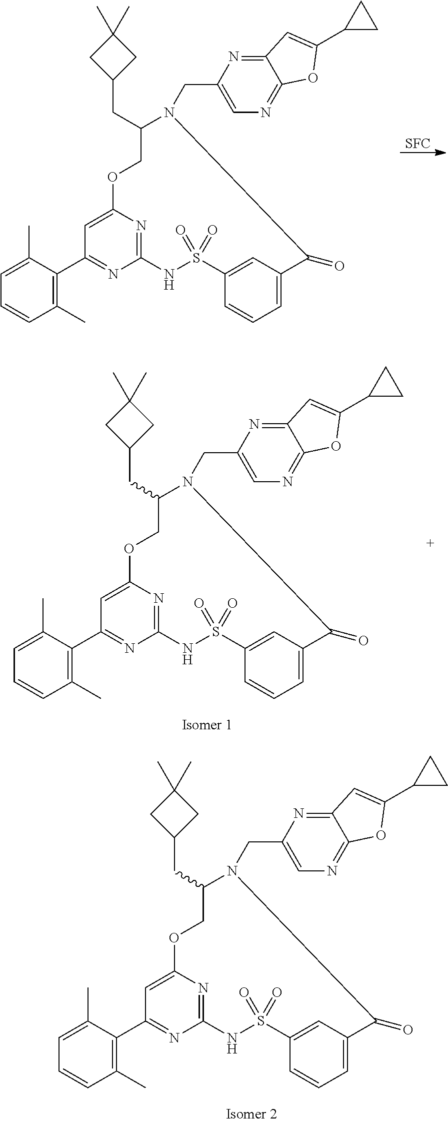

The enantiomers of 12-[(6-cyclopropylfuro[2,3-b]pyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (66.3 mg, 0.09570 mmol) were separated by chiral SFC using a ChiralCel OD (21.2×250 mm, 5 μM) column at 40° C. Mobile phase was 22% MeOH (20 mM NH₃), 78% CO₂ at 70 mL/min flow. Concentration of the sample was 30 mg/mL in methanol (no modifier), injection volume was 500 μL, outlet pressure at 157 bar, and detection wavelength 215 nm. Each enantiomer was collected separately and the resulting products were each further purified by preparative HPLC (1-99% ACN in water, HCl modifier, 15 minute run) to give Enantiomer 1 (Peak 1 SFC), 12-[(6-cyclopropylfuro[2,3-b]pyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (24.5 mg, 37%) ¹H NMR (400 MHz, Chloroform-d) δ 8.66 (t, J=1.8 Hz, 1H), 8.34 (s, 1H), 8.10 (dt, J=8.2, 1.3 Hz, 1H), 7.84 (dt, J=7.9, 1.3 Hz, 1H), 7.67 (t, J=7.8 Hz, 1H), 7.22 (t, J=7.6 Hz, 1H), 7.07 (d, J=7.6 Hz, 2H), 6.62 (s, 1H), 6.33-6.21 (m, 1H), 5.39-5.34 (m, 1H), 5.31 (d, J=15.1 Hz, 1H), 4.35-4.20 (m, 2H), 4.08-3.97 (m, 1H), 2.17-2.09 (m, 1H), 2.04 (s, 6H), 1.96-1.80 (m, 3H), 1.65-1.59 (m, 1H), 1.42-1.35 (m, 2H), 1.19-1.12 (m, 4H), 1.05 (s, 3H), 0.91 (s, 3H), 0.85-0.78 (m, 1H). Sulfonamide NH not visible. ESI-MS m/z calc. 692.2781, found 693.9 (M+1)⁺; Retention time: 2.12 minutes. and Enantiomer 2 (Peak 2 SFC), 12-[(6-cyclopropylfuro[2,3-b]pyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (23.3 mg, 35%) ¹H NMR (400 MHz, Chloroform-d) δ 8.65 (t, J=1.9 Hz, 1H), 8.34 (s, 1H), 8.10 (d, J=7.9 Hz, 1H), 7.84 (d, J=7.7, 1.8 Hz, 1H), 7.66 (t, J=7.8 Hz, 1H), 7.21 (t, J=7.6 Hz, 1H), 7.06 (d, J=7.6 Hz, 2H), 6.62 (s, 1H), 6.26 (s, 1H), 5.39-5.27 (m, 2H), 4.34-4.17 (m, 2H), 4.03 (t, J=11.2 Hz, 1H), 2.17-2.10 (m, 1H), 2.00 (s, 6H), 1.97-1.80 (m, 3H), 1.62 (ddd, J=14.4, 9.0, 3.2 Hz, 1H), 1.45-1.35 (m, 2H), 1.20-1.10 (m, 4H), 1.05 (s, 3H), 0.91 (s, 3H), 0.85-0.80 (m, 1H). Sulfonamide NH not visible ESI-MS m/z calc. 692.2781, found 693.7 (M+1)⁺; Retention time: 2.12 minutes. LC method A.

### Example 83: Preparation of Compound I-169

### Step 1: 3,6-Dibromopyrazine-2-carboxylic acid

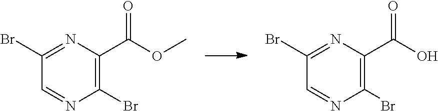

A stirred solution of methyl 3,6-dibromopyrazine-2-carboxylate (10 g, 33.793 mmol) in a mixture of tetrahydrofuran (50 mL) and water (25 mL) was cooled down to 0° C. and lithium hydroxide hydrate (2.2 g, 52.426 mmol) was added to the mixture. The reaction was stirred at 0° C. for 2 hours then quenched by the addition of an aqueous solution of 1 N hydrochloric acid (50 mL). The layers were separated and the aqueous layer was extracted with ethyl acetate (5×50 mL) and the combined organic layers were washed with brine (200 mL), dried over magnesium sulfate, filtered and concentrated under reduced pressure to afford crude 3,6-dibromopyrazine-2-carboxylic acid (9.28 g, 94%) as a blackish oil that was directly used in the next Step without further purification. ESI-MS m/z calc. 279.8483, found 281.0 (M+1)⁺; Retention time: 0.91 minutes; LC method I.

### Step 2: 3,6-Dibromo-N-methoxy-N-methyl-pyrazine-2-carboxamide

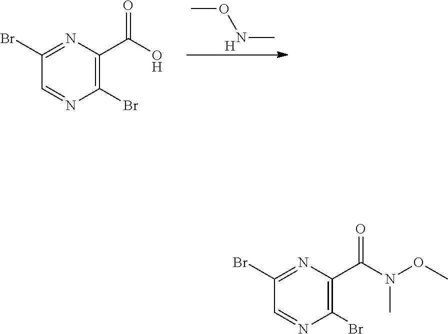

A stirred solution of 3,6-dibromopyrazine-2-carboxylic acid (9.28 g, 31.703 mmol) in anhydrous DMF (100 mL) was cooled down to 0° C. and N-methoxymethanamine (hydrochloride salt) (6.2 g, 63.561 mmol), HATU (14.5 g, 38.135 mmol) and triethylamine (10.309 g, 14.2 mL, 101.88 mmol) were successively added. The reaction was stirred at 0° C. for 30 minutes then stirred overnight at room temperature. The reaction was diluted with ethyl acetate (250 mL) and water (150 mL). A solution of 1 N hydrochloric acid was added and the mixture was vigorously stirred for 15 minutes. The layers were separated, and the aqueous layer was extracted with ethyl acetate (3×150 mL) and 2-methyltetrahydrofuran (2×150 mL). The combined organic layers were washed with brine (2×200 mL), then dried over magnesium sulfate, filtered and concentrated under reduced pressure. The crude was then purified by reverse-phase chromatography (column: 275 HP Gold; gradient: 5 to 100% methanol in water containing 0.1% v/v of formic acid, 16 CV). The desired fractions were concentrated under reduced pressure and the remaining water was neutralized using a saturated aqueous solution of sodium bicarbonate (150 mL), then extracted with dichloromethane (5×100 mL). The combined organic layers were washed with brine (250 mL), dried over magnesium sulfate, filtered, and concentrated under reduced pressure to afford 3,6-dibromo-N-methoxy-N-methyl-pyrazine-2-carboxamide (7.69 g, 56%) ¹H NMR (400 MHz, CDCl₃) δ 8.51 (s, 1H), 3.63 (s, 3H), 3.42 (s, 3H). ESI-MS m/z calc. 322.8905, found 323.8 (M+1)⁺; Retention time: 1.6 minutes; LC method I.

### Step 3: 3,6-Dibromopyrazine-2-carbaldehyde

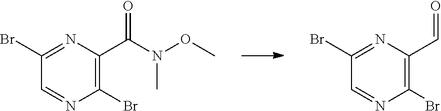

A stirred solution of 3,6-dibromo-N-methoxy-N-methyl-pyrazine-2-carboxamide (7.7 g, 17.724 mmol) in anhydrous toluene (150 mL) was cooled down to −78° C. A solution of DIBAL-H in toluene (27 mL of 1 M, 27.000 mmol) was added dropwise over 30 minutes and the reaction was stirred at −78° C. for 5 hours. The reaction was quenched using a saturated aqueous solution of sodium and potassium tartrate (250 mL), then allowed to reach room temperature and stirred overnight at room temperature. The layers were separated, and the aqueous layer was extracted with ethyl acetate (4×50 mL). The combined organic layers were washed with brine (250 mL), dried over magnesium sulfate, filtered, and concentrated under reduced pressure to afford crude 3,6-dibromopyrazine-2-carbaldehyde (8.2 g, 100%) as a dark orange oil which was directly engaged in the next step without further purification. ESI-MS m/z calc. 263.8534, found 265.1 (M+1)⁺; Retention time: 2.1 minutes; LC method K.

### Step 4: 5-Bromo-1H-pyrazolo[3,4-b]pyrazine

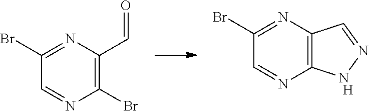

To a stirred solution of crude 3,6-dibromopyrazine-2-carbaldehyde (8.2 g, 17.733 mmol) in isopropanol (150 mL) was added a solution of hydrazine in ethanol (90 mL of 1 M, 90.000 mmol). The reaction was then stirred overnight at 110° C. The reaction was then cooled down to room temperature and concentrated under reduced pressure the crude was then dissolved in a 1:1 mixture of 2-methyltetrahydrofuran and water (800 mL). The biphasic mixture was then vigorously stirred for 30 minutes. The layers were then separated, and the aqueous layer was extracted with 2-methyltetrahydrofuran (5×200 mL). The combined organic layers were washed with brine (500 mL), dried over magnesium sulfate, filtered and concentrated under reduced pressure to afford a dark orange solid. The crude was purified by reverse-phase chromatography on C₁₈ (column: 275 g HP Gold C₁₈; gradient: 5 to 100% methanol in water containing 0.1% v/v of formic acid, 18 CV). The desired fractions were concentrated under reduced pressure and the residual water was co-evaporated with methanol (6×50 mL) then acetonitrile (3×15 mL) and the residue was dried under high vacuum to afford 5-bromo-1H-pyrazolo[3,4-b]pyrazine (1.306 g, 36%) as a brown solid. ¹H NMR (400 MHz, DMSO-d6) δ 14.23 (br. s, 1H), 8.74 (s, 1H), 8.47 (s, 1H). ESI-MS m/z calc. 197.9541, found 199.0 (M+1)⁺; Retention time: 1.36 minutes; LC method I.

### Step 5: 5-Bromo-2-isopropyl-pyrazolo[3,4-b]pyrazine

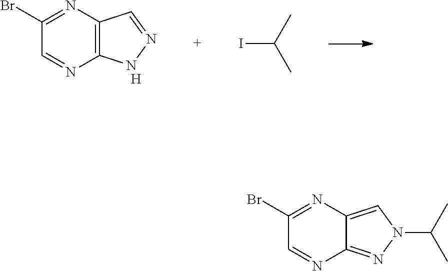

A stirred solution of crude 5-bromo-1H-pyrazolo[3,4-b]pyrazine (1.61 g, 7.4267 mmol) in anhydrous tetrahydrofuran (25 mL) under nitrogen was cooled down to 0° C. Then a solution of NaHMDS in tetrahydrofuran (15 mL of 1 M, 15.000 mmol) was added, followed by 2-iodopropane (4.2575 g, 2.5 mL, 25.045 mmol). The reaction was stirred at 0° C. for 30 minutes then heated to 70° C. and stirred overnight at 70° C. The reaction was cooled down to room temperature and concentrated under reduced pressure to afford a blackish oil that was purified by reverse-phase chromatography on C₁₈ (column: 275 g HP Gold; gradient: 10 to 100% acetonitrile in water containing 0.1% v/v of formic acid; 16 CV). The desired fractions were concentrated under reduced pressure, then dried under high vacuum to afford 5-bromo-2-isopropyl-pyrazolo[3,4-b]pyrazine (405 mg, 22%) as a brown solid. ESI-MS m/z calc. 240.00105, found 241.2 (M+1)⁺; Retention time: 1.59 minutes; LC method I.

### Step 6: 2-Isopropyl-5-vinyl-pyrazolo[3,4-b]pyrazine

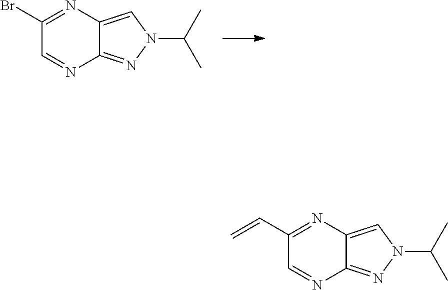

To a stirred solution of 5-bromo-2-isopropyl-pyrazolo[3,4-b]pyrazine (405 mg, 1.5976 mmol) in anhydrous toluene (4 mL) were added, tributyl(vinyl)stannane (1.02 g, 3.2167 mmol), lithium chloride (205 mg, 4.8356 mmol) and copper iodide (61 mg, 0.3203 mmol). Nitrogen was bubbled to the mixture for 10 minutes then Pd(PPh₃)₄ (185 mg, 0.1601 mmol) was added. The tube was then sealed, and the reaction was stirred overnight at 90° C. The reaction was then cooled down to room temperature, filtered on a pad of Celite, washed with ethyl acetate (2×15 mL) and concentrated under reduced pressure. The crude was then purified by reverse-phase chromatography on C₁₈ (column: 50 g HP Gold C₁₈; gradient: 5 to 100% acetonitrile in water containing 0.1% v/v, 16 CV). The desired fractions were concentrated under reduced pressure and the remaining water was neutralized using a saturated aqueous solution of sodium bicarbonate (15 mL). The aqueous layer was then extracted with ethyl acetate (3×20 mL) and the combined organic layers were washed with brine (60 mL), dried over magnesium sulfate, filtered and concentrated under reduced pressure to afford 2-isopropyl-5-vinyl-pyrazolo[3,4-b]pyrazine (152 mg, 50%) as a dark yellow oil. ¹H NMR (400 MHz, CDCl₃) δ 8.80 (s, 1H), 8.18 (s, 1H), 6.95 (dd, J=17.9, 11.0 Hz, 1H), 6.31 (d, J=17.9 Hz, 1H), 5.66 (d, J=11.2 Hz, 1H), 4.85 (spt, J=6.7 Hz, 1H), 1.70 (d, J=6.8 Hz, 6H). ESI-MS m/z calc. 188.1062, found 189.2 (M+1)⁺; Retention time: 1.46 minutes; LC method I.

### Step 7: 2-Isopropylpyrazolo[3,4-b]pyrazine-5-carbaldehyde

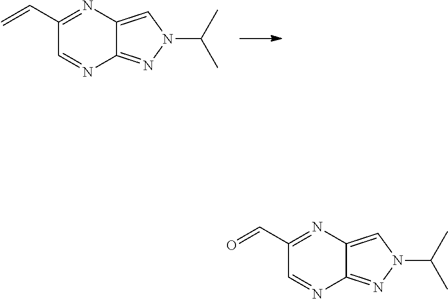

To stirred solution of 2-isopropyl-5-vinyl-pyrazolo[3,4-b]pyrazine (152 mg, 0.8003 mmol) in a mixture of tetrahydrofuran (3 mL) and water (1.5 mL) at 0° C. were successively added 4-methylmorpholine N-oxide (188 mg, 1.6048 mmol) and a solution of osmium (VIII) tetroxide in tert-butanol (400 mg, 2.5% w/w, 0.0393 mmol). The reaction was stirred at 0° C. for 5 minutes after which time sodium periodate (700 mg, 3.2727 mmol) was added. The reaction was stirred at room temperature after which time supplementary osmium (VIII) tetroxide solution in tert-butanol (400 mg, 2.5% w/w, 0.0393 mmol), 4-methylmorpholine N-oxide (188 mg, 1.6048 mmol) and sodium periodate (700 mg, 3.2727 mmol) were added. The reaction was stirred at room temperature for 2 days. The reaction was then dissolved in water (50 mL) and the aqueous layer was extracted with ethyl acetate (3×30 mL). The combined organic layers were washed with brine (50 mL), dried over magnesium sulfate, filtered, and concentrated under reduced pressure. The crude was then purified by flash chromatography on silica gel (Column: 12 g HP Gold; gradient: 0 to 100% ethyl acetate in heptanes, 16 CV). The desired fractions were concentrated under reduced pressure to afford 2-isopropylpyrazolo[3,4-b]pyrazine-5-carbaldehyde (102 mg, 67%) as an off-white solid. ¹H NMR (400 MHz, CDCl₃) δ 10.15 (s, 1H), 9.27 (s, 1H), 8.47 (s, 1H), 4.94 (spt, J=6.7 Hz, 1H), 1.76 (d, J=6.6 Hz, 6H). ESI-MS m/z calc. 190.08546, found 191.1 (M+1)⁺; Retention time: 2.18 minutes; LC method K.

### Step 8: 3-[[4-(2,6-Dimethylphenyl)-6-[(2R)-2-[(2-isopropylpyrazolo[3,4-b]pyrazin-5-yl)methylamino]-4,4-dimethyl-pentoxy]pyrimidin-2-yl]sulfamoyl]benzoic acid

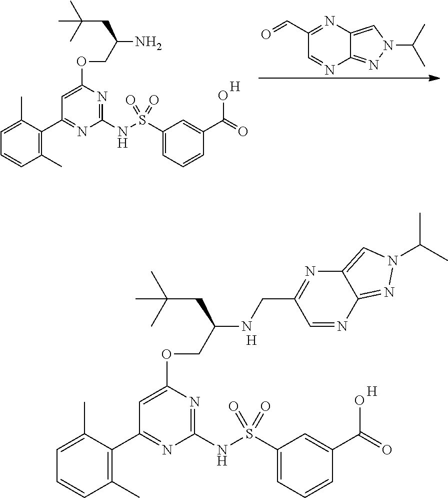

A stirred solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (303 mg, 0.5336 mmol) and 2-isopropylpyrazolo[3,4-b]pyrazine-5-carbaldehyde (102 mg, 0.5336 mmol) in anhydrous dichloromethane (13 mL) under nitrogen atmosphere was cooled down to 0° C. Then sodium triacetoxyborohydride (510 mg, 2.4063 mmol) was added and the reaction was stirred at 0° C. for 1 hour. The reaction was then quenched by the addition of an aqueous solution of 1 N hydrochloric acid (15 mL). The biphasic mixture was vigorously stirred at 0° C. for 30 minutes, then the layers were separated, and the aqueous layer was extracted with 2-methyltetrahydrofuran (5×20 mL). The combined organic layers were washed with water (30 mL) and brine (30 mL), dried over magnesium sulfate, filtered, and concentrated under reduced pressure to afford an off-white solid. The solid was co-evaporated with heptanes (3×15 mL) to remove the residual acetic acid and afforded crude 3-[[4-(2,6-dimethylphenyl)-6-[(2R)-2-[(2-isopropylpyrazolo[3,4-b]pyrazin-5-yl)methylamino]-4,4-dimethyl-pentoxy]pyrimidin-2-yl]sulfamoyl]benzoic acid (hydrochloride salt) (461 mg, 75%) as an off-white powder that was directly engaged in the next Step without further purification. ESI-MS m/z calc. 686.29987, found 687.2 (M+1)⁺; Retention time: 1.58 minutes; LC method I.

### Step 9: (11R)-6-(2,6-Dimethylphenyl)-11-(2,2-dimethylpropyl)-12-[(2-isopropylpyrazolo[3,4-b]pyrazin-5-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-169

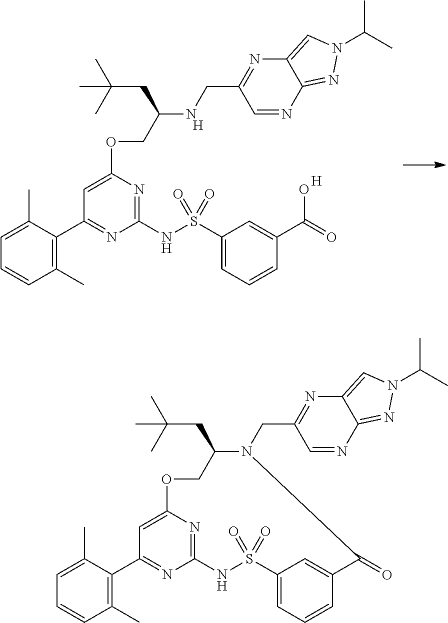

A stirred solution of crude 3-[[4-(2,6-dimethylphenyl)-6-[(2R)-2-[(2-isopropylpyrazolo[3,4-b]pyrazin-5-yl)methylamino]-4,4-dimethyl-pentoxy]pyrimidin-2-yl]sulfamoyl]benzoic acid (hydrochloride salt) (461 mg, 0.3984 mmol) in anhydrous DMF (50 mL) under nitrogen atmosphere was cooled down to 0° C. Then N-methylmorpholine (552.00 mg, 600 μL, 5.4574 mmol) and 2-chloro-4,6-dimethoxy-1,3,5-triazine (210 mg, 1.1961 mmol) were added. The reaction was stirred at 0° C. for 30 minutes then stirred overnight at room temperature. The solvent was evaporated under reduced pressure and water (25 mL) and ethyl acetate (50 mL) were added. The biphasic medium was vigorously stirred for 30 minutes, then the layers were separated, and the aqueous layer was extracted with ethyl acetate (3×30 mL) and 2-methyltetrahydrofuran (2×30 mL). The combined organic layers were washed with brine, dried over magnesium sulfate, filtered and concentrated under reduced pressure to afford an off-white foam. The crude was then purified by reverse-phase chromatography on C₁₈ (column: 50 g HP Gold Cis, gradient: 5 to 100% acetonitrile in 10 mM ammonium bicarbonate buffer pH=10, 20 CV). The desired fractions were concentrated under reduced pressure to afford a white residue that was purified again by reverse phase chromatography on C₁₈ (column: 50 g HP Gold Cis, gradient: 20 to 50% acetonitrile in 10 mM ammonium bicarbonate buffer pH=10, 12 CV). The desired fractions were concentrated under reduced pressure and the remaining water was co-evaporated with acetonitrile (6×5 mL), and the residue was freeze-dried to afford (11R)-6-(2,6-dimethylphenyl)-11-(2,2-dimethylpropyl)-12-[(2-isopropylpyrazolo[3,4-b]pyrazin-5-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (25.2 mg, 9%) as a white fluffy powder. ¹H NMR (400 MHz, CDCl₃) δ 9.01-8.95 (m, 1H), 8.80 (s, 1H), 8.25 (s, 1H), 8.11 (d, J=8.1 Hz, 1H), 7.87 (d, J=7.8 Hz, 1H), 7.66 (t, J=7.7 Hz, 1H), 7.25-7.16 (m, 1H), 7.13-7.04 (m, 2H), 6.27 (s, 1H), 5.56 (dd, J=11.2, 4.4 Hz, 1H), 5.36 (d, J=15.6 Hz, 1H), 4.86 (spt, J=6.8 Hz, 1H), 4.40 (d, J=15.7 Hz, 1H), 4.28-4.18 (m, 1H), 4.12-4.05 (m, 1H), 2.02 (s, 6H), 1.78 (dd, J=15.3, 8.7 Hz, 1H), 1.70 (d, J=6.8 Hz, 6H), 1.60 (d, J=14.7 Hz, 1H, overlapped with water), 0.66 (s, 9H). (1H missing, labile H). ESI-MS m/z calc. 668.2893, found 669.3 (M+1)⁺; Retention time: 3.02 minutes; LC method K.

### Example 84: Preparation of Compound I-175

### Step 1: 5-Bromo-N3-methyl-pyrazine-2,3-diamine

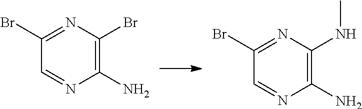

A suspension of 3,5-dibromopyrazin-2-amine (1 g, 3.9542 mmol) in MeNH₂ in water (10 mL of 40% w/v, 128.80 mmol) was heated in a microwave reactor at 130° C. for 1 h. The reaction was cooled to room temperature and extracted with DCM (3×15 mL). The organic phase was dried over MgSO₄, filtered and concentrated in vacuo to provide 5-bromo-N3-methyl-pyrazine-2,3-diamine (802 mg, 100%) as a tan solid. ¹H NMR (400 MHz, Chloroform-d) δ 7.43 (s, 1H), 4.48 (s, 1H), 4.24 (s, 2H), 2.99 (d, J=5.0 Hz, 3H). ESI-MS m/z calc. 201.98541, found 203.1 (M+1)⁺; Retention time: 2.4 minutes; LC method D.

### Step 2: 5-Bromo-2-cyclopropyl-3-methyl-imidazo[4,5-b]pyrazine

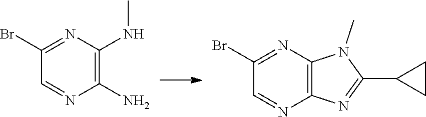

To a pressure vessel containing a solution of cyclopropanecarboxylic anhydride (6.0675 g, 5.36 mL, 39.358 mmol) in cyclopropanecarboxylic acid (25 mL) was added 5-bromo-N3-methyl-pyrazine-2,3-diamine (4 g, 19.701 mmol) and the mixture was sealed under N2 gas and stirred at room temperature for 10 minutes. The reaction was heated in a 130° C. oil bath for 1 h. cyclopropanecarboxylic anhydride (3.0338 g, 2.68 mL, 19.679 mmol) was added. The reaction was heated in a 130° C. oil bath for 12 h. The reaction was cooled down to room temperature and diluted with EtOAc (300 mL), washed with 2M NaOH (2×100 mL), brine, dried over MgSO₄, filtered and concentrated in vacuo. The residue was purified by flash chromatography (loaded onto a 120 g SiO₂ cartridge with toluene, and eluted with 0-40% EtOAc in Hexanes over a 60 min gradient) to provide 5-bromo-2-cyclopropyl-3-methyl-imidazo[4,5-b]pyrazine (2.9 g, 58%) as a yellow crystalline solid. ¹H NMR (500 MHz, Chloroform-d) δ 8.46 (s, 1H), 3.91 (s, 3H), 2.05 (tt, J=8.2, 8.2, 4.8, 4.8 Hz, 1H), 1.46-1.41 (m, 2H), 1.28-1.23 (m, 2H). ESI-MS m/z calc. 252.00105, found 253.3 (M+1)⁺; Retention time: 2.75 minutes; LC method D.

### Step 3: Methyl 2-cyclopropyl-3-methyl-imidazo[4,5-b]pyrazine-5-carboxylate

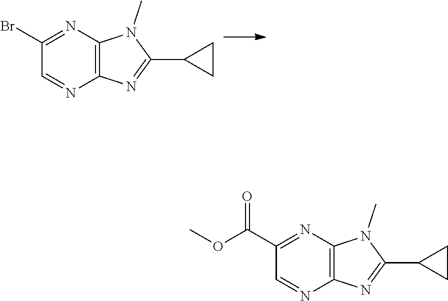 [SMILES: Cn1c(C2CC2)nc2ncc(Br)nc21 ; COC(=O)c1cnc2nc(C3CC3)n(C)c2n1]

To a solution of 5-bromo-2-cyclopropyl-1-methyl-imidazo[4,5-b]pyrazine (2.6 g, 10.273 mmol) in MeOH (52 mL) was added Pd(dppf)Cl₂·DCM (755 mg, 0.9245 mmol) and then TEA (20.328 g, 28 mL, 200.89 mmol). The vessel was bubbled with N2 gas for 5 minutes. The vessel was then sealed and pressurized with CO gas (100 psi), vented 3 times, then re-pressurized at 100 psi and heated at 80° C. for 30 minutes. The reaction was cooled down to room temperature and stirred under 100 psi of CO pressure for 12 h. The pressure was released, and the mixture was concentrated in vacuo. The reaction was diluted with EtOAc (50 mL), washed with saturated aqueous NH₄Cl (30 mL), brine, dried over MgSO₄, filtered, and concentrated in vacuo. The residue was purified by flash chromatography (dissolved in DCM and dry loaded onto a 120 g SiO₂ cartridge and eluted with 0-70% EtOAc in Hexanes over a 60 min gradient) to provide methyl 2-cyclopropyl-1-methyl-imidazo[4,5-b]pyrazine-5-carboxylate (1.79 g, 75%) as an off white crystalline solid. ESI-MS m/z calc. 232.09602, found 233.3 (M+1)⁺; Retention time: 2.1 minutes; LC method D.

### Step 4: 2-Cyclopropyl-3-methyl-imidazo[4,5-b]pyrazine-5-carbaldehyde

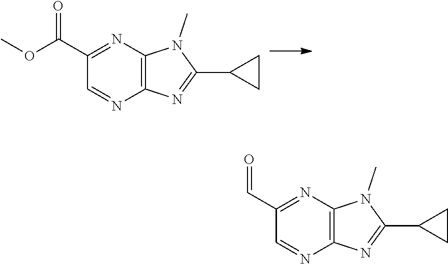 [SMILES: COC(=O)c1cnc2nc(C3CC3)n(C)c2n1 ; Cn1c(C2CC2)nc2ncc(C=O)nc21]

A solution of methyl 2-cyclopropyl-3-methyl-imidazo[4,5-b]pyrazine-5-carboxylate (1.23 g, 5.2963 mmol) in DCM (18.2 mL) was cooled in a −78° C. bath for 15 minutes then DIBAL in DCM (8 mL of 1 M, 8.0000 mmol) was added dropwise. The reaction was stirred at this temperature for 25 min at −78° C. The reaction was quenched with MeOH—H₂O (1:1, 20 mL) at −78° C. and stirred 10 minutes then warmed to room temperature and further stirred for 30 min. The reaction was concentrated in vacuo, then DCM (30 mL) and 1 M HCl (15 mL) were added. The layers were separated and the aqueous was extracted with DCM (3×15 mL). The combined organics were washed with brine (50 mL), dried over MgSO₄, filtered, and concentrated in vacuo. The residue was purified by flash chromatography (dry loaded onto an 80 g SiO₂ cartridge, and eluted with 0-70% EtOAc in Hexanes over a 60 min gradient) to provide 2-cyclopropyl-3-methyl-imidazo[4,5-b]pyrazine-5-carbaldehyde (569.8 mg, 52%) as colorless solid. ¹H NMR (500 MHz, Chloroform-d) δ 10.17 (s, 1H), 9.07 (s, 1H), 4.02 (s, 3H), 2.19-2.10 (m, 1H), 1.58-1.51 (m, 2H), 1.39-1.32 (m, 2H). ESI-MS m/z calc. 202.08546, found 203.2 (M+1)⁺; Retention time: 0.99 minutes; LC method H.

### Step 5: 3-[[4-[(2R)-3-(1-Bicyclo[1.1.1]pentanyl)-2-[(2-cyclopropyl-3-methyl-imidazo[4,5-b]pyrazin-5-yl)methylamino]propoxy]-6-(2,6-dimethylphenyl)pyrimidin-2-yl]sulfamoyl]benzoic acid

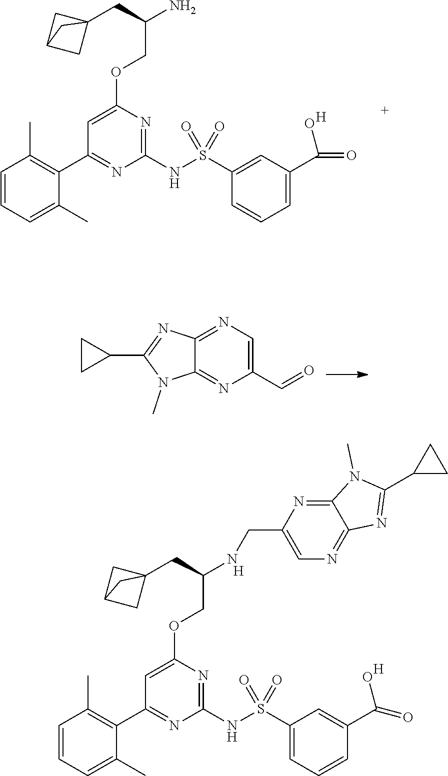

A 4 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (60 mg, 0.1073 mmol), 2-cyclopropyl-3-methyl-imidazo[4,5-b]pyrazine-5-carbaldehyde (22.1 mg, 0.1093 mmol), anhydrous DCM (0.55 mL), and acetic acid (12.3 μL, 0.2163 mmol). The mixture was cooled down in an ice bath. DIEA (56.1 μL, 0.3221 mmol) was added, followed by sodium triacetoxyborohydride (115.5 mg, 0.5450 mmol), and the reaction was vigorously stirred at 0° C. for 3 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-[(2R)-3-(1-bicyclo[1.1.1]pentanyl)-2-[(2-cyclopropyl-3-methyl-imidazo[4,5-b]pyrazin-5-yl)methylamino]propoxy]-6-(2,6-dimethylphenyl)pyrimidin-2-yl]sulfamoyl]benzoic acid (hydrochloride salt) (21.5 mg, 25%) as a white solid. ESI-MS m/z calc. 708.28424, found 709.5 (M+1)⁺; Retention time: 1.3 minutes. LC method A.

### Step 6: (11R)-11-(1-Bicyclo[1.1.1]pentanylmethyl)-12-[(2-cyclopropyl-3-methyl-imidazo[4,5-b]pyrazin-5-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-175

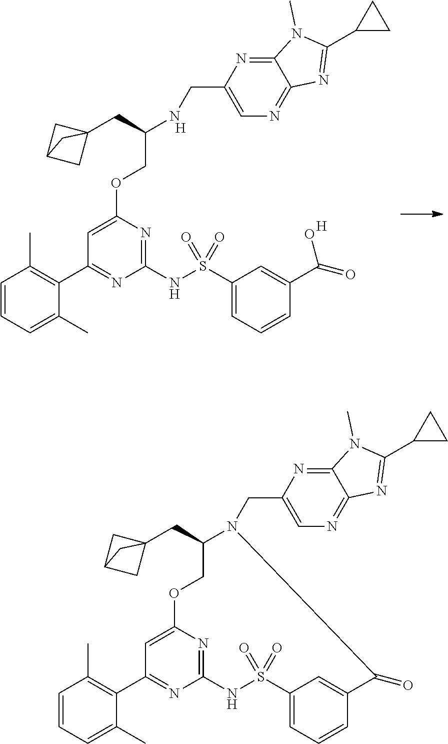

3-[[4-[(2R)-3-(1-Bicyclo[1.1.1]pentanyl)-2-[(2-cyclopropyl-3-methyl-imidazo[4,5-b]pyrazin-5-yl)methylamino]propoxy]-6-(2,6-dimethylphenyl)pyrimidin-2-yl]sulfamoyl]benzoic acid (hydrochloride salt) (21.5 mg, 0.02654 mmol) was combined with CDMT (9.8 mg, 0.05582 mmol) in DMF (2.4 mL) and cooled to 0° C. N-methylmorpholine (10.0 μL, 0.09096 mmol) was added by syringe and the reaction was stirred at 0° C. for 30 minutes. The ice bath was then removed and stirring was continued for an additional 16 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO and MeOH, filtered, and purified by reverse phase HPLC (1-99% acetonitrile/5 mM aqueous HCl over 25 min) to give (11R)-11-(1-bicyclo[1.1.1]pentanylmethyl)-12-[(2-cyclopropyl-3-methyl-imidazo[4,5-b]pyrazin-5-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.3 mg, 28%) as a white solid. ¹H NMR (400 MHz, CDCl₃) δ 8.96 (t, J=1.8 Hz, 1H), 8.59 (s, 1H), 8.01 (d, J=7.9 Hz, 1H), 7.87 (dt, J=7.7, 1.4 Hz, 1H), 7.66 (t, J=7.8 Hz, 1H), 7.23 (t, J=7.6 Hz, 1H), 7.05 (d, J=7.6 Hz, 2H), 6.18 (s, 1H), 5.60-5.53 (m, 1H), 5.45 (d, J=16.0 Hz, 1H), 4.35 (d, J=16.1 Hz, 1H), 4.06 (s, 3H), 4.03-3.86 (m, 2H), 2.47 (s, 1H), 2.21-2.09 (m, 1H), 2.05 (s, 6H), 2.01-1.91 (m, 1H), 1.86-1.79 (m, 1H), 1.72-1.67 (m, 1H), 1.66-1.57 (m, 7H), 1.37-1.32 (m, 2H). ESI-MS m/z calc. 690.2737, found 691.2 (M+1)⁺; Retention time: 1.65 minutes. LC method A.

### Example 85: Preparation of Compound I-176 and Compound I-177

### Step 1: 2-Amino-3-isopropyl-4-methyl-pentan-1-ol

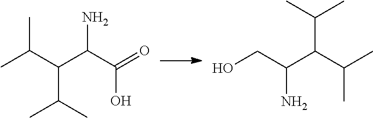

To a solution of 2-amino-3-isopropyl-4-methyl-pentanoic acid (hydrochloride salt) (250 mg, 1.192 mmol) in Tetrahydrofuran (2.500 mL) was added Borane Tetrahydrofuran Complex (4.5 mL of 1 M, 4.500 mmol) at 0° C. dropwise. The reaction was allowed to warm to room temperature and stirred for 24 h. The reaction was quenched with slow addition of methanol (30 mL) and then co-evaporated with methanol (3×50 mL) and DCE (1×5 mL). The crude material was then suspended in DCM (50 mL) and acidified with HCl (1.5 mL of 4 M, 6.000 mmol) in dioxane to give 2-amino-3-isopropyl-4-methyl-pentan-1-ol (hydrochloride salt) (230 mg, 99%). ESI-MS m/z calc. 159.16231, found 160.1 (M+1)⁺; Retention time: 0.56 minutes; LC method B.

### Step 2: 3-[[4-(2-Amino-3-isopropyl-4-methyl-pentoxy)-6-(2,6-dimethylphenyl)pyrimidin-2-yl]sulfamoyl]benzoic acid

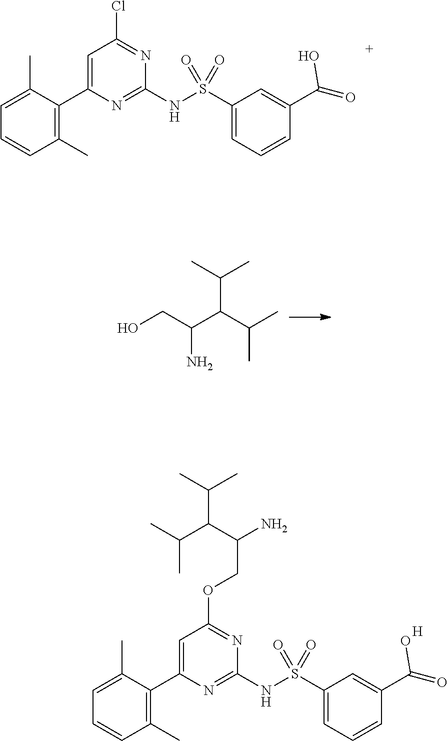

3-[[4-Chloro-6-(2,6-dimethylphenyl)pyrimidin-2-yl]sulfamoyl]benzoic acid (300 mg, 0.7179 mmol) was combined with 2-amino-3-isopropyl-4-methyl-pentan-1-ol (hydrochloride salt) (155 mg, 0.7919 mmol) in THE (1.8 mL) and stirred for 5 minutes. Sodium tert-butoxide (350 mg, 3.642 mmol) was then added in one portion, and the reaction mixture was stirred vigorously at room temperature for 30 minutes. The temperature was increased to 45° C. for 30 minutes, then the reaction was cooled to room temperature and partitioned between 1 M HCl and ethyl acetate. The layers were separated and the aqueous was extracted an additional 2× with ethyl acetate. The aqueous layer was then diluted with brine and extracted a further 2× ethyl acetate. The combined organics were washed with brine, dried over sodium sulfate and concentrated to give 3-[[4-(2-amino-3-isopropyl-4-methyl-pentoxy)-6-(2,6-dimethylphenyl)pyrimidin-2-yl]sulfamoyl]benzoic acid (hydrochloride salt) (449.0 mg, 98%), which was used without further purification. ESI-MS m/z calc. 540.24066, found 541.6 (M+1)⁺; Retention time: 0.51 minutes; LC method B.

### Step 3: 3-[[4-[2-[(6-Cyclopropylfuro[2,3-b]pyrazin-2-yl)methylamino]-3-isopropyl-4-methyl-pentoxy]-6-(2,6-dimethylphenyl)pyrimidin-2-yl]sulfamoyl]benzoic acid

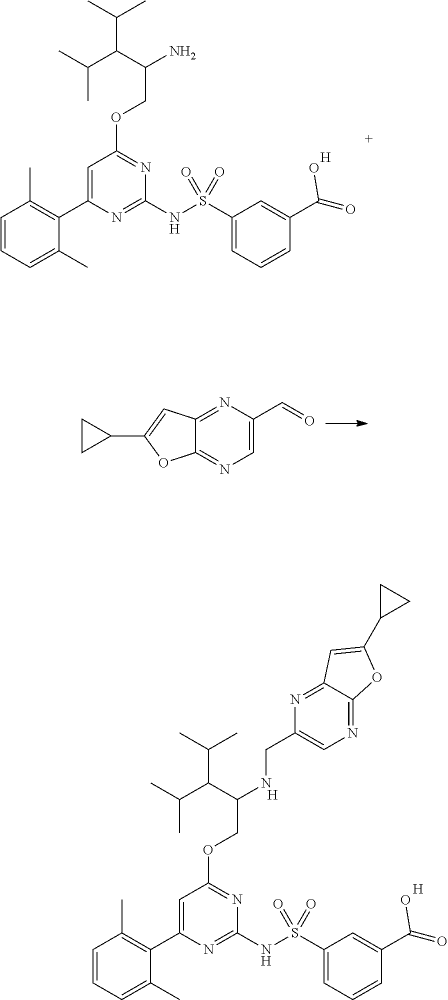

3-[[4-(2-Amino-3-isopropyl-4-methyl-pentoxy)-6-(2,6-dimethylphenyl)pyrimidin-2-yl]sulfamoyl]benzoic acid (hydrochloride salt) (100 mg, 0.1559 mmol) was combined with 6-cyclopropylfuro[2,3-b]pyrazine-2-carbaldehyde (32 mg, 0.1700 mmol) in DCM (0.4 mL) and stirred for 20 minutes at room temperature. Sodium triacetoxyborohydride (33 mg, 0.1557 mmol) was added, and the reaction was stirred for 15 minutes at room temperature, before an additional portion of sodium triacetoxyborohydride (99 mg, 0.4671 mmol) was added. After 2 hours at room temperature the reaction mixture was quenched with 3M HCl then diluted with methanol and DMSO until the reaction mixture became homogeneous. It was then filtered and purified by preparative HPLC (1-99% ACN in water, HCl modifier, 15 minute run) to give 3-[[4-[2-[(6-cyclopropylfuro[2,3-b]pyrazin-2-yl)methylamino]-3-isopropyl-4-methyl-pentoxy]-6-(2,6-dimethylphenyl)pyrimidin-2-yl]sulfamoyl]benzoic acid (hydrochloride salt) (45.2 mg, 39%). ESI-MS m/z calc. 712.3043, found 713.7 (M+1)⁺; Retention time: 0.6 minutes; LC method B.

### Step 4: 12-[(6-Cyclopropylfuro[2,3-b]pyrazin-2-yl)methyl]-6-(2,6-dimethylphenyl)-11-(1-isopropyl-2-methyl-propyl)-2,2-dioxo-9-oxa-2λ⁶-thia-3,5,12,19-tetrazatricyclo[12.3.1.14,8]nonadeca-1(18),4(19),5,7,14,16-hexaen-13-one

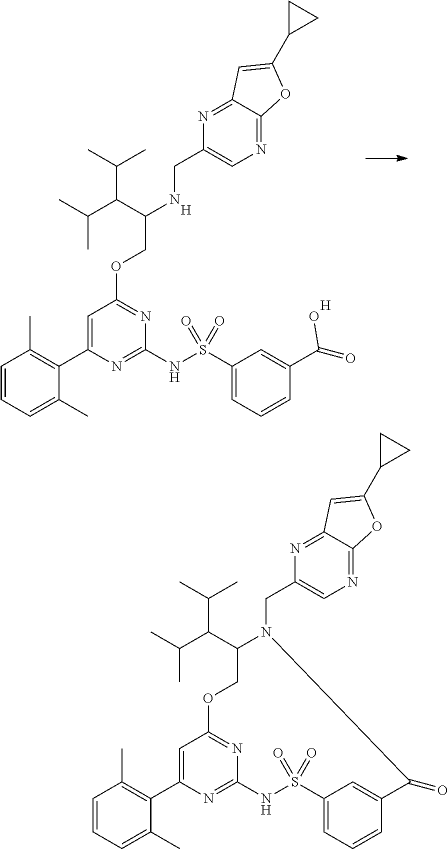

3-[[4-[2-[(6-Cyclopropylfuro[2,3-b]pyrazin-2-yl)methylamino]-3-isopropyl-4-methyl-pentoxy]-6-(2,6-dimethylphenyl)pyrimidin-2-yl]sulfamoyl]benzoic acid (hydrochloride salt) (45 mg, 0.06005 mmol) was combined with CDMT (13 mg, 0.07404 mmol) in DMF (4 mL) and cooled to 0° C. in an ice water bath. N-methylmorpholine (40 μL, 0.3638 mmol) was added and the reaction was allowed to warm to room temperature as the ice melted. After 16 hours an additional portion of CDMT (8 mg, 0.04557 mmol) was added. After a total of 68 hours from the first addition of N-methylmorpholine the reaction mixture was partitioned between 1 M HCl and ethyl acetate. The layers were separated and the aqueous was extracted an additional 2× with ethyl acetate. The combined organics were washed with brine and dried over sodium sulfate. The resulting crude material was purified by chromatography on silica gel, eluting with a 0-15% methanol in DCM gradient to give 12-[(6-cyclopropylfuro[2,3-b]pyrazin-2-yl)methyl]-6-(2,6-dimethylphenyl)-11-(1-isopropyl-2-methyl-propyl)-2,2-dioxo-9-oxa-2λ⁶-thia-3,5,12,19-tetrazatricyclo[12.3.1.14,8]nonadeca-1(18),4(19),5,7,14,16-hexaen-13-one (12.8 mg, 31%) ESI-MS m/z calc. 694.29376, found 695.7 (M+1)⁺; Retention time: 0.85 minutes. LC method B.

### Step 5: 12-[(6-Cyclopropylfuro[2,3-b]pyrazin-2-yl)methyl]-6-(2,6-dimethylphenyl)-11-(1-isopropyl-2-methyl-propyl)-2,2-dioxo-9-oxa-2λ⁶-thia-3,5,12,19-tetrazatricyclo[12.3.1.14,8]nonadeca-1(18),4(19),5,7,14,16-hexaen-13-one, Compound I-176, enantiomer 1, and 12-[(6-cyclopropylfuro[2,3-b]pyrazin-2-yl)methyl]-6-(2,6-dimethylphenyl)-11-(1-isopropyl-2-methyl-propyl)-2,2-dioxo-9-oxa-2λ⁶-thia-3,5,12,19-tetrazatricyclo[12.3.1.14,8]nonadeca-1(18),4(19),5,7,14,16-hexaen-3-one, enantiomer 2, Compound I-177

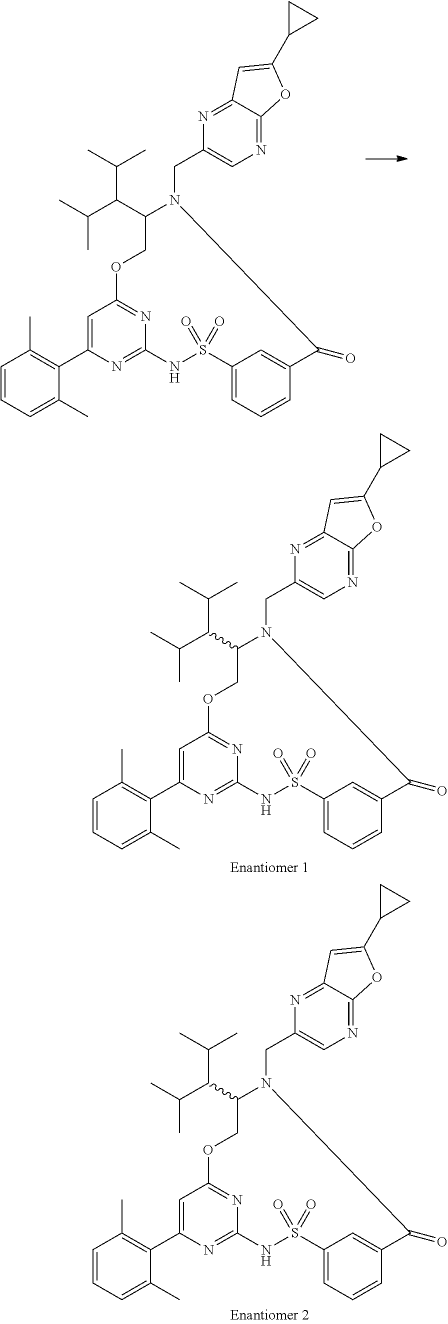

The enantiomers of 12-[(6-cyclopropylfuro[2,3-b]pyrazin-2-yl)methyl]-6-(2,6-dimethylphenyl)-11-(1-isopropyl-2-methyl-propyl)-2,2-dioxo-9-oxa-2λ⁶-thia-3,5,12,19-tetrazatricyclo[12.3.1.14,8]nonadeca-1(18),4(19),5,7,14,16-hexaen-13-one (12.8 mg, 0.01842 mmol) were separated by chiral SFC using a ChiralPak AS (21.2×250 mm, 5 μM) column at 40° C. Mobile phase was 20% MeOH (20 mM NH₃), 80% CO₂ at 70 mL/min flow. Concentration of the sample was 12.8 mg/mL in methanol (no modifier), injection volume was 500 μL, outlet pressure at 158 bar, and detection wavelength 210 nm. Each enantiomer was collected separately and the resulting products were each further purified by preparative HPLC (1-99% ACN in water, HCl modifier, 15 minute run) to give enantiomer 1 (Peak 1 SFC), 12-[(6-cyclopropylfuro[2,3-b]pyrazin-2-yl)methyl]-6-(2,6-dimethylphenyl)-11-(1-isopropyl-2-methyl-propyl)-2,2-dioxo-9-oxa-2λ⁶-thia-3,5,12,19-tetrazatricyclo[12.3.1.14,8]nonadeca-1(18),4(19),5,7,14,16-hexaen-13-one (2.1 mg, 16%) ESI-MS m/z calc. 694.29376, found 695.5 (M+1)⁺; Retention time: 2.28 minutes. and enantiomer 2 (Peak 2 SFC), 12-[(6-cyclopropylfuro[2,3-b]pyrazin-2-yl)methyl]-6-(2,6-dimethylphenyl)-11-(1-isopropyl-2-methyl-propyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.8 mg, 14%) ESI-MS m/z calc. 694.29376, found 695.5 (M+1)⁺; Retention time: 2.21 minutes. LC method A.

### Example 86: Preparation of Compound I-187

### Step 1: 6-Chloro-3-(3,3-dimethylbut-1-ynyl)pyrazin-2-amine

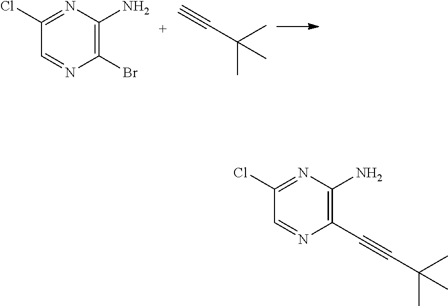

A solution of 3-bromo-6-chloro-pyrazin-2-amine (1 g, 4.7015 mmol) and 3,3-dimethylbut-1-yne (662.35 mg, 7.8214 mmol) in triethylamine (10 mL) was sparged with nitrogen for five minutes, then bis(triphenylphosphine)palladium(II) chloride (330.03 mg, 0.4702 mmol) and CuI (89.550 mg, 0.4702 mmol) were added. The mixture was again sparged with nitrogen for 5 minutes and then the vial was sealed, and the resulting mixture was allowed to stir at room temperature for 30 minutes. The reaction mixture was diluted with 20 mL of ethyl acetate and filtered through a Celite pad. The pad was washed with an additional 20 mL of ethyl acetate and the solids were discarded. The combined filtrates were extracted with 3×40 mL of water and 1×40 mL of brine. The aqueous phases were discarded, and the organic phase was dried over sodium sulfate and concentrated under vacuum to obtain a dark solid. The crude product was purified by silica gel chromatography using a 80 g column and eluting with a gradient of 5-50% ethyl acetate in hexane to afford after evaporation 6-chloro-3-(3,3-dimethylbut-1-ynyl)pyrazin-2-amine (880 mg, 83%) as a beige solid. ¹H NMR (500 MHz, Chloroform-d) δ 7.85 (d, 1H), 5.08 (s, 2H), 1.37 (s, 9H). ESI-MS m/z calc. 209.07198, found 209.9 (M+1)⁺; Retention time: 4.5 minutes; LC method D.

### Step 2: 6-tert-Butyl-3-chloro-5H-pyrrolo[2,3-b]pyrazine

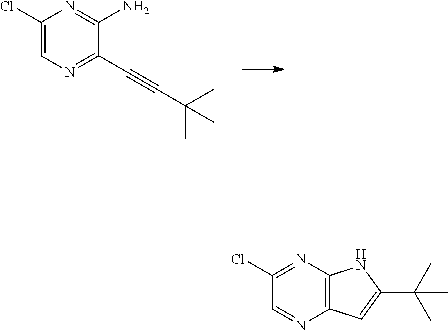

To a solution of 6-chloro-3-(3,3-dimethylbut-1-ynyl)pyrazin-2-amine (880 mg, 4.1970 mmol) in tert-butanol (9 mL) was added potassium tert-butoxide (2.1 g, 2.3282 mL, 18.715 mmol). The mixture was stirred at 80° C. for 16 hours then cooled to room temperature. The reaction mixture was concentrated in vacuo to dryness, then water (20 mL) was added, and the resulting slurry was stirred at room temperature for one hour. The mixture was filtered, and the filtrate was discarded. The solid was triturated with 2 mL of acetonitrile, then the resulting slurry was filtered. The filtrate was discarded and the solid was dried in vacuo to afford an off-white solid that was purified using a 80 g column and eluting with a gradient of 5-50% ethyl acetate in hexane to afford after evaporation 6-tert-butyl-3-chloro-5H-pyrrolo[2,3-b]pyrazine (700 mg, 79%) as a white solid. ¹H NMR (500 MHz, DMSO-d6) δ 12.12 (s, 1H), 8.34 (s, 1H), 6.39 (s, 1H), 1.35 (s, 9H). ESI-MS m/z calc. 209.07198, found 209.9 (M+1)⁺; Retention time: 4.05 minutes; LC method D.

### Step 3: 6-tert-Butyl-3-chloro-5-methyl-pyrrolo[2,3-b]pyrazine

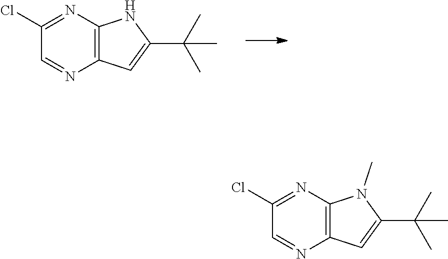

To a solution of 6-tert-butyl-3-chloro-5H-pyrrolo[2,3-b]pyrazine (690 mg, 3.2908 mmol) in DMF (8 mL) at 0° C. was added 60% (w/v) sodium hydride (366 mg, 60% w/w, 9.1509 mmol). The mixture was stirred at 0° C. for fifteen minutes, then dimethyl sulfate (877 mg, 0.6594 mL, 6.9530 mmol) was added. The mixture was stirred at 0° C. for five minutes, then at room temperature for one hour. The mixture was poured into 35 mL of water and extracted with 3×35 mL of ethyl acetate. The aqueous phase was discarded, and the combined organic phases were extracted with 3×35 mL of water and 25 mL of brine. The aqueous phases were discarded, and the organic phase was dried over sodium sulfate, filtered, and concentrated in vacuo. The crude product was purified by silica gel chromatography using an 80 g silica gel column eluted with a gradient of 5-50% ethyl acetate in hexane to afford after evaporation 6-tert-butyl-3-chloro-5-methyl-pyrrolo[2,3-b]pyrazine (460 mg, 59%) ¹H NMR (500 MHz, Chloroform-d) δ 8.33 (s, 1H), 6.49 (s, 1H), 3.98 (s, 3H), 1.50 (s, 9H). ESI-MS m/z calc. 223.08763, found 224.1 (M+1)⁺; Retention time: 5.06 minutes.

### Step 4: Methyl 6-tert-butyl-5-methyl-pyrrolo[2,3-b]pyrazine-3-carboxylate

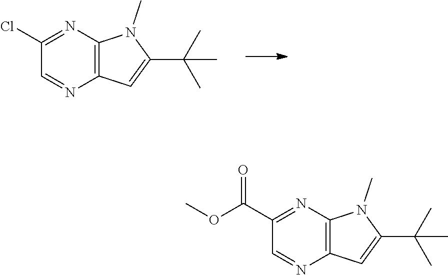 [SMILES: Cn1c(C(C)(C)C)cc2ncc(Cl)nc21 ; COC(=O)c1cnc2cc(C(C)(C)C)n(C)c2n1]

A mixture of 6-tert-butyl-3-chloro-5-methyl-pyrrolo[2,3-b]pyrazine (1.43 g, 6.3924 mmol), triethylamine (1.34 g, 1.8457 mL, 13.242 mmol), and Pd(dppf)Cl₂. DCM (261 mg, 0.3196 mmol) in methanol (30 mL) was stirred at 100° C. for 18 hours under 50 psi of carbon monoxide. The reaction mixture was allowed to cool to room temperature and was filtered through a Celite pad. The solids were discarded, and the filtrate was concentrated in vacuo to obtain a dark oil. The material was dissolved in 40 mL of 1:1 ethyl acetate:water, then the phases were separated, and the aqueous phase was discarded. The organic phase was dried over sodium sulfate: the solids were removed by filtration and discarded, and the filtrate was concentrated in vacuo to obtain a dark oil that was purified by silica gel chromatography (40 g column, 5-55% ethyl acetate:hexane gradient, 1 hour) to obtain methyl 6-tert-butyl-5-methyl-pyrrolo[2,3-b]pyrazine-3-carboxylate (1.29 g, 82%) ¹H NMR (500 MHz, Chloroform-d) δ 9.16 (s, 1H), 6.59 (s, 1H), 4.09 (s, 3H), 4.03 (s, 3H), 1.53 (s, 9H). ESI-MS m/z calc. 247.13208, found 248.2 (M+1)⁺; Retention time: 4.24 minutes; LC method D.

### Step 5: (6-tert-Butyl-5-methyl-pyrrolo[2,3-b]pyrazin-3-yl)methanol

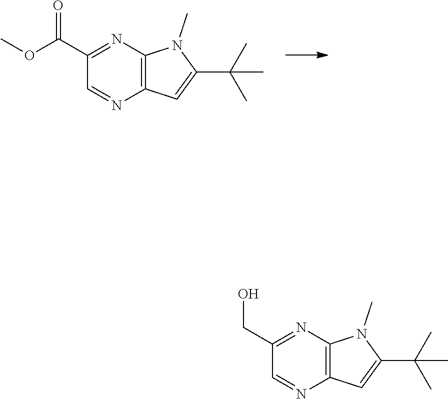 [SMILES: COC(=O)c1cnc2cc(C(C)(C)C)n(C)c2n1 ; Cn1c(C(C)(C)C)cc2ncc(CO)nc21]

A solution of 1.0 M LAH in THE (2.9802 mL of 1 M, 2.9802 mmol) was cooled in an ice water bath, then a solution of methyl 6-tert-butyl-5-methyl-pyrrolo[2,3-b]pyrazine-3-carboxylate (670 mg, 2.7093 mmol) in THE (3 mL) was added dropwise and the resulting mixture was allowed to stir in the ice water bath for 30 minutes. The reaction was quenched with a small amount of water, then was concentrated in vacuo to obtain an orange oil. The crude product was dissolved in a small amount of DCM and was dried over sodium sulfate. The solids were filtered away and discarded and the filtrate was loaded onto a 25 g silica gel column and eluted using a 5-95% gradient of ethyl acetate in hexane to obtain 6-tert-butyl-5-methyl-pyrrolo[2,3-b]pyrazin-3-yl)methanol (407 mg, 66%) as a white solid. ¹H NMR (500 MHz, Chloroform-d) δ 9.12 (s, 1H), 8.02 (s, 1H), 5.65 (s, 2H), 4.79 (s, 3H), 3.83 (s, 1H), 2.28 (s, 9H). ESI-MS m/z calc. 219.13716, found 220.0 (M+1)⁺; Retention time: 2.71 minutes; LC method D.

### Step 6: 6-tert-Butyl-5-methyl-pyrrolo[2,3-b]pyrazine-3-carbaldehyde

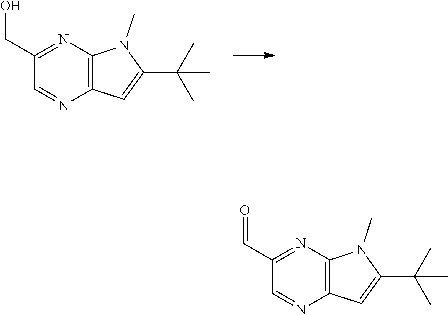 [SMILES: Cn1c(C(C)(C)C)cc2ncc(CO)nc21 ; Cn1c(C(C)(C)C)cc2ncc(C=O)nc21]

Dess-Martin periodinane (1.3 g, 3.0650 mmol) was added to a solution of (6-tert-butyl-5-methyl-pyrrolo[2,3-b]pyrazin-3-yl)methanol (650 mg, 2.9642 mmol) in DCM (6 mL). The resulting solution was stirred at room temperature for 45 minutes. The reaction mixture was diluted with 25 mL of dichloromethane, then was quenched with 10 mL of saturated sodium bicarbonate. The phases were separated, and the aqueous phase was extracted with 2×10 mL of dichloromethane. The combined organic phases were extracted with 2×10 mL of water, and the organic phase was dried over anhydrous sodium sulfate. The solids were removed by filtration, the filtrate was concentrated in vacuo and the residue was purified on a 40 g silica gel column by flash chromatography (5-55% gradient of ethyl acetate:hexane). The pure fractions were dried in vacuo to afford 6-tert-butyl-5-methyl-pyrrolo[2,3-b]pyrazine-3-carbaldehyde (333 mg, 52%). ¹H NMR (500 MHz, Chloroform-d) δ 10.16 (s, 1H), 9.03 (s, 1H), 6.62 (s, 1H), 4.10 (s, 3H), 1.55 (s, 9H). ESI-MS m/z calc. 217.1215, found 218.2 (M+1)⁺; Retention time: 2.05 minutes; LC method H.

### Step 7: 3-[[4-[(2R)-2-[(6-tert-Butyl-5-methyl-pyrrolo[2,3-b]pyrazin-3-yl)methylamino]-3-(1-methylcyclopropyl)propoxy]-6-(2,6-dimethylphenyl)pyrimidin-2-yl]sulfamoyl]benzoic acid

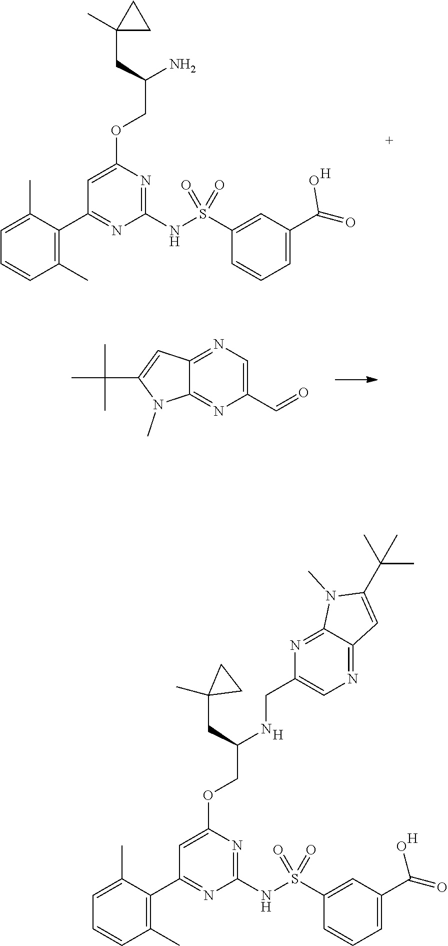

3-[[4-[(2R)-2-Amino-3-(1-methylcyclopropyl)propoxy]-6-(2,6-dimethylphenyl)pyrimidin-2-yl]sulfamoyl]benzoic acid (hydrochloride salt) (50 mg, 0.09140 mmol) was combined with 6-tert-butyl-5-methyl-pyrrolo[2,3-b]pyrazine-3-carbaldehyde (22 mg, 0.1013 mmol) in DCM (0.5 mL) and acetic acid (10 μL, 0.1758 mmol) was added. The reaction mixture was cooled to 0° C. in an ice bath. DIEA (40 μL, 0.2296 mmol) was added followed by sodium triacetoxyborohydride (97 mg, 0.4577 mmol). After 3.5 hours at 0° C. the reaction mixture was quenched with 0.2 mL 3M HCl then diluted with methanol and DMSO until the reaction mixture became homogeneous. It was filtered and purified by preparative HPLC (1-99% ACN in water, HCl modifier, 15 minute run) to give 3-[[4-[(2R)-2-[(6-tert-butyl-5-methyl-pyrrolo[2,3-b]pyrazin-3-yl)methylamino]-3-(1-methylcyclopropyl)propoxy]-6-(2,6-dimethylphenyl)pyrimidin-2-yl]sulfamoyl]benzoic acid (hydrochloride salt) (59 mg, 86%). ESI-MS m/z calc. 711.3203, found 712.9 (M+1)⁺; Retention time: 0.59 minutes; LC method B.

### Step 8: (11R)-12-[(6-tert-Butyl-5-methyl-pyrrolo[2,3-b]pyrazin-3-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-187

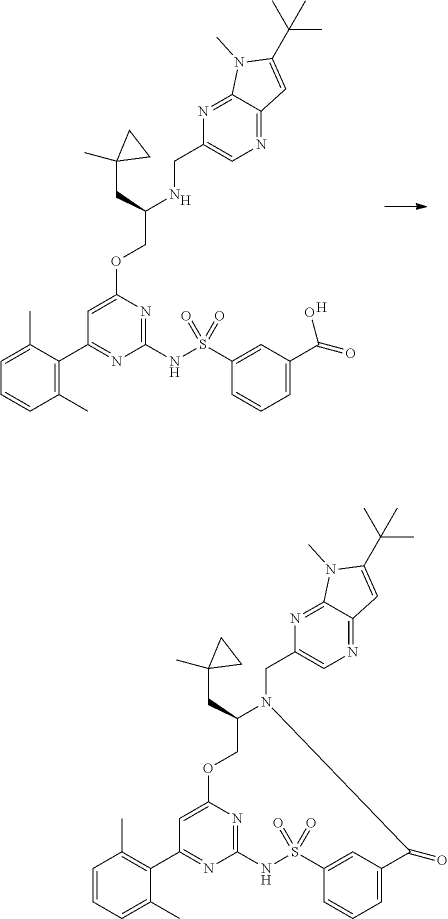

3-[[4-[(2R)-2-[(6-tert-Butyl-5-methyl-pyrrolo[2,3-b]pyrazin-3-yl)methylamino]-3-(1-methylcyclopropyl)propoxy]-6-(2,6-dimethylphenyl)pyrimidin-2-yl]sulfamoyl]benzoic acid (hydrochloride salt) (59 mg, 0.07884 mmol) was combined with CDMT (17 mg, 0.09683 mmol) in DMF (3.5 mL). The reaction mixture was cooled to 0° C. in an ice bath and N-methylmorpholine (60 μL, 0.5457 mmol) was added. The reaction mixture was stirred at 0° C. for 30 minutes then warmed to room temperature and stirred for an additional 3.5 hours. The reaction mixture was then filtered and purified by preparative HPLC (10-99% ACN in water, HCl modifier, 15 minute run) to give (11R)-12-[(6-tert-butyl-5-methyl-pyrrolo[2,3-b]pyrazin-3-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (22.7 mg, 41%). ESI-MS m/z calc. 693.30975, found 694.7 (M+1)⁺; Retention time: 1.81 minutes; LC method A. ¹H NMR (400 MHz, Chloroform-d) δ 8.98 (t, J=1.8 Hz, 1H), 8.51 (s, 1H), 8.05 (dt, J=7.9, 1.5 Hz, 1H), 7.89 (dt, J=7.7, 1.4 Hz, 1H), 7.65 (t, J=7.8 Hz, 1H), 7.22 (t, J=7.6 Hz, 1H), 7.08 (d, J=7.6 Hz, 2H), 6.69 (s, 1H), 6.25 (s, 1H), 5.58 (dd, J=11.1, 4.3 Hz, 1H), 5.40 (d, J=15.6 Hz, 1H), 4.46 (t, J=10.6 Hz, 1H), 4.29 (d, J=15.8 Hz, 1H), 4.15 (s, 3H), 4.08 (t, J=11.4 Hz, 1H), 2.04 (s, 6H), 1.86 (d, J=14.8 Hz, 1H), 1.52 (s, 9H), 1.47-1.42 (m, 1H), 0.53 (s, 3H), 0.38 (dt, J=9.7, 5.0 Hz, 1H), 0.28 (dt, J=9.7, 5.0 Hz, 1H), 0.16 (dt, J=9.3, 4.6 Hz, 1H), 0.08-0.02 (m, 1H). Sulfonamide NH Not visible

### Example 87: Preparation of Compound I-188

### Step 1: 3-[[4-[(2R)-2-[(6-tert-Butyl-5-methyl-pyrrolo[2,3-b]pyrazin-3-yl)methylamino]-4,4-dimethyl-pentoxy]-6-(2,6-dimethylphenyl)-5-methyl-pyrimidin-2-yl]sulfamoyl]benzoic acid

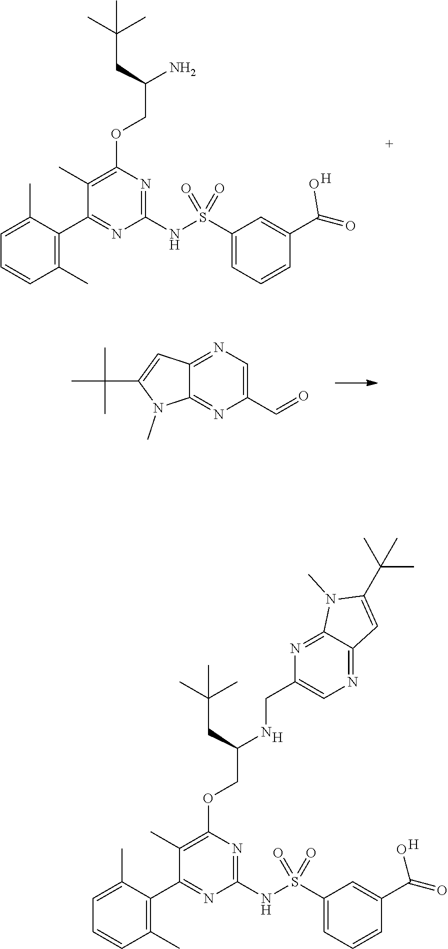

3-[[4-[(2R)-2-Amino-4,4-dimethyl-pentoxy]-6-(2,6-dimethylphenyl)-5-methyl-pyrimidin-2-yl]sulfamoyl]benzoic acid (hydrochloride salt) (50 mg, 0.08879 mmol) was combined with 6-tert-butyl-5-methyl-pyrrolo[2,3-b]pyrazine-3-carbaldehyde (21 mg, 0.09666 mmol) in DCM (0.5 mL) and acetic acid (10 μL, 0.1758 mmol) was added. The reaction mixture was cooled to 0° C. in an ice bath. DIEA (40 μL, 0.2296 mmol) was added followed by sodium triacetoxyborohydride (94 mg, 0.4435 mmol). After 3.5 hours at 0° C. the reaction mixture was quenched with 0.2 mL 3M HCl then diluted with methanol and DMSO until the reaction mixture became homogeneous. It was filtered and purified by preparative HPLC (1-99% ACN in water, HCl modifier, 15 minute run) to give 3-[[4-[(2R)-2-[(6-tert-butyl-5-methyl-pyrrolo[2,3-b]pyrazin-3-yl)methylamino]-4,4-dimethyl-pentoxy]-6-(2,6-dimethylphenyl)-5-methyl-pyrimidin-2-yl]sulfamoyl]benzoic acid (hydrochloride salt) (53 mg, 78%). ESI-MS m/z calc. 727.35156, found 728.9 (M+1)⁺; Retention time: 0.62 minutes; LC method B.

### Step 2: (11R)-12-[(6-tert-Butyl-5-methyl-pyrrolo[2,3-b]pyrazin-3-yl)methyl]-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-188

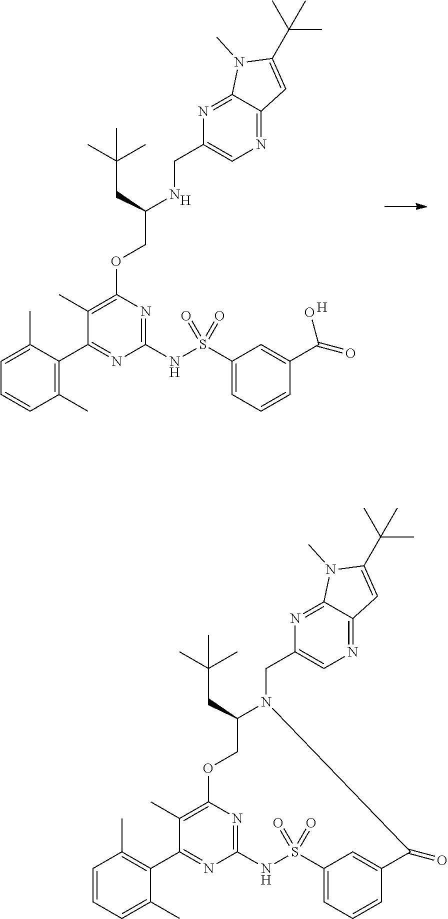

3-[[4-[(2R)-2-[(6-tert-Butyl-5-methyl-pyrrolo[2,3-b]pyrazin-3-yl)methylamino]-4,4-dimethyl-pentoxy]-6-(2,6-dimethylphenyl)-5-methyl-pyrimidin-2-yl]sulfamoyl]benzoic acid (hydrochloride salt) (53 mg, 0.06934 mmol) was combined with CDMT (15 mg, 0.08543 mmol) in DMF (3.5 mL). The reaction mixture was cooled to 0° C. in an ice bath and N-methylmorpholine (50 μL, 0.4548 mmol) was added. The reaction mixture was stirred at 0° C. for 30 minutes then warmed to room temperature and stirred for an additional 3.5 hours. The reaction mixture was then filtered and purified by preparative HPLC (10-99% ACN in water, HCl modifier, 15 minute run) to give (11R)-12-[(6-tert-butyl-5-methyl-pyrrolo[2,3-b]pyrazin-3-yl)methyl]-6-(2,6-dimethylphenyl)-11-(2,2-dimethylpropyl)-7-methyl-2,2-dioxo-9-oxa-2λ⁶-thia-3,5,12,19-tetrazatricyclo[12.3.1.14,8]nonadeca-1(18),4(19),5,7,14,16-hexaen-13-one (28.5 mg, 57%). ESI-MS m/z calc. 709.341, found 710.9 (M+1)⁺; Retention time: 1.91 minutes; LC method A. ¹H NMR (400 MHz, Chloroform-d) δ 9.06 (t, J=1.8 Hz, 1H), 8.50 (s, 1H), 8.11 (dt, J=8.0, 1.3 Hz, 1H), 7.87 (dt, J=7.7, 1.3 Hz, 1H), 7.66 (t, J=7.8 Hz, 1H), 7.22 (t, J=7.6 Hz, 1H), 7.11 (d, J=7.6 Hz, 1H), 7.06 (d, J=7.6 Hz, 1H), 6.61 (s, 1H), 5.66 (dd, J=11.0, 4.1 Hz, 1H), 5.42 (d, J=15.4 Hz, 1H), 4.44 (d, J=15.6 Hz, 1H), 4.32-4.24 (m, 1H), 4.18-4.07 (m, 4H), 2.04 (s, 3H), 1.84 (s, 3H), 1.77 (d, J=8.3 Hz, 1H), 1.74 (s, 3H), 1.64-1.60 (m, 1H), 1.51 (s, 9H), 0.65 (s, 9H). Sulfonamide NH not visible

### Example 88: Preparation of Compound I-191

### Step 1: 3-[[4-[(2R)-2-Amino-3-phenyl-propoxy]-6-(2,6-dimethylphenyl)pyrimidin-2-yl]sulfamoyl]benzoic acid

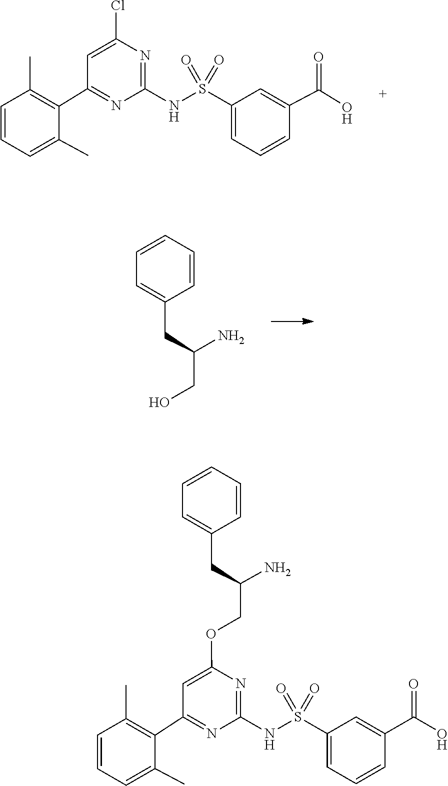 [SMILES: Cc1cccc(C)c1-c1cc(Cl)nc(NS(=O)(=O)c2cccc(C(=O)O)c2)n1 ; N[C@@H](CO)Cc1ccccc1 ; Cc1cccc(C)c1-c1cc(OC[C@H](N)Cc2ccccc2)nc(NS(=O)(=O)c2cccc(C(=O)O)c2)n1]

3-[[4-Chloro-6-(2,6-dimethylphenyl)pyrimidin-2-yl]sulfamoyl]benzoic acid (300 mg, 0.7179 mmol) was combined with (2R)-2-amino-3-phenyl-propan-1-ol (120 mg, 0.7936 mmol) in THE (1.8 mL) and stirred at room temperature until the reaction mixture became homogeneous. Sodium tert-Butoxide (345 mg, 3.590 mmol) was added, and the reaction became warm to the touch. Stirring was continued for 20 minutes with no external heating. The reaction mixture was then partitioned between 1 M HCl and ethyl acetate. The layers were separated and the aqueous was extracted 2× ethyl acetate, then diluted with an equal volume of brine and extracted an additional 2× ethyl acetate. The combined organics were washed with brine, dried over sodium sulfate and concentrated to give 3-[[4-[(2R)-2-amino-3-phenyl-propoxy]-6-(2,6-dimethylphenyl)pyrimidin-2-yl]sulfamoyl]benzoic acid (hydrochloride salt) (440 mg, 97%) ESI-MS m/z calc. 532.17804, found 533.6 (M+1)⁺; Retention time: 0.46 minutes. LC method B.

### Step 2: 3-[[4-[(2R)-2-[(6-Cyclopropylfuro[2,3-b]pyrazin-2-yl)methylamino]-3-phenyl-propoxy]-6-(2,6-dimethylphenyl)pyrimidin-2-yl]sulfamoyl]benzoic acid

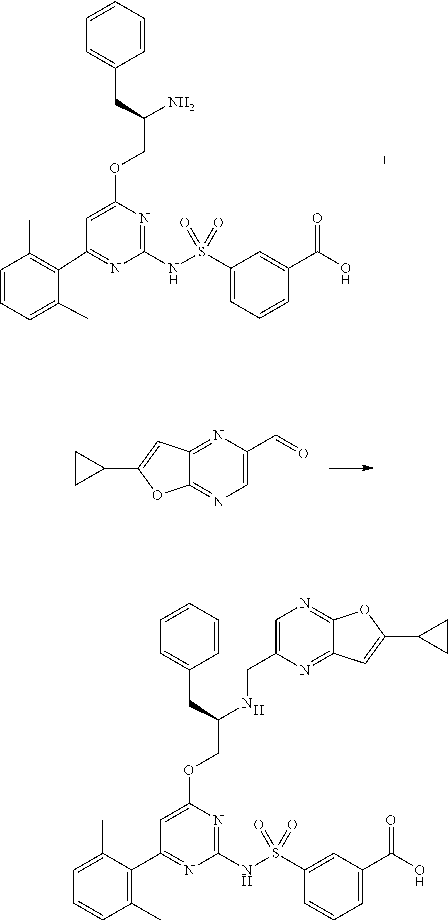

3-[[4-[(2R)-2-Amino-3-phenyl-propoxy]-6-(2,6-dimethylphenyl)pyrimidin-2-yl]sulfamoyl]benzoic acid (hydrochloride salt) (50 mg, 0.07908 mmol) was combined with 6-cyclopropylfuro[2,3-b]pyrazine-2-carbaldehyde (16 mg, 0.08502 mmol) in DCM (0.3 mL) and stirred for 15 minutes at room temperature. Sodium triacetoxyborohydride (17 mg, 0.08021 mmol) was added and the reaction mixture was stirred for an additional 15 minutes before a second portion of sodium triacetoxyborohydride (50 mg, 0.2359 mmol) was added. After an additional 30 minutes the reaction was quenched with 0.2 mL 3M HCl then diluted with methanol and DMSO until it became homogeneous, was filtered, and purified by preparative HPLC (1-99% ACN in water, HCl modifier, 15 minute run) to give 3-[[4-[(2R)-2-[(6-cyclopropylfuro[2,3-b]pyrazin-2-yl)methylamino]-3-phenyl-propoxy]-6-(2,6-dimethylphenyl)pyrimidin-2-yl]sulfamoyl]benzoic acid (hydrochloride salt) (31 mg, 56%). ESI-MS m/z calc. 704.2417, found 705.6 (M+1)⁺; Retention time: 0.57 minutes; LC method B.

### Step 3: (11R)-11-Benzyl-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-191

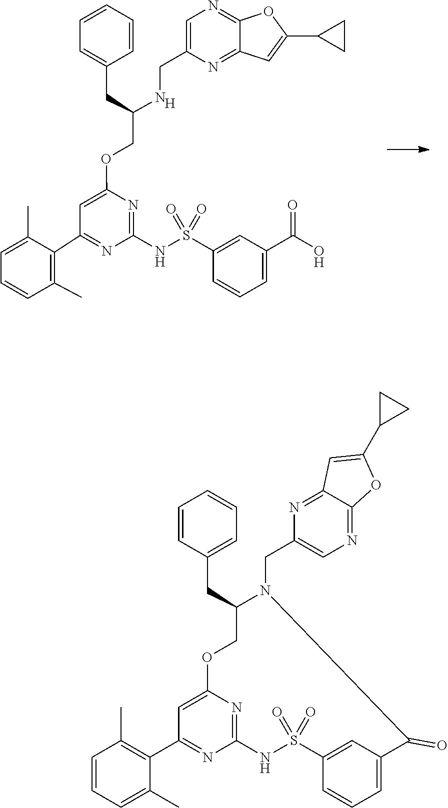

3-[[4-[(2R)-2-[(6-Cyclopropylfuro[2,3-b]pyrazin-2-yl)methylamino]-3-phenyl-propoxy]-6-(2,6-dimethylphenyl)pyrimidin-2-yl]sulfamoyl]benzoic acid (hydrochloride salt) (31 mg, 0.04182 mmol) was combined with CDMT (9 mg, 0.05126 mmol) in DMSO then cooled to 0° C. in an ice bath. N-methylmorpholine (30 μL, 0.2729 mmol) was added and after stirring for 15 minutes at 0° C. the reaction was allowed to warm to room temperature then stirred for 2.5 hours. Volatiles were then removed by rotary evaporation and the resulting residue was dissolved in 1:1 methanol/DMSO, filtered and purified by preparative HPLC (10-99% ACN in water, HCl modifier, 15 minute) to give (11R)-11-benzyl-12-[(6-cyclopropylfuro[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (17.3 mg, 60%). ESI-MS m/z calc. 686.23114, found 687.7 (M+1)⁺; Retention time: 1.89 minutes; LC method A. ¹H NMR (400 MHz, Chloroform-d) δ 8.57 (s, 1H), 8.31 (s, 1H), 8.03 (d, J=7.9 Hz, 1H), 7.50 (t, J=7.8 Hz, 1H), 7.25-7.16 (m, 5H), 7.08 (d, J=7.6 Hz, 2H), 6.85-6.72 (m, 2H), 6.63 (s, 1H), 6.29 (s, 1H), 5.42 (d, J=9.0 Hz, 1H), 5.27 (d, J=14.8 Hz, 1H), 4.54-4.30 (m, 3H), 3.07-2.90 (m, 2H), 2.13 (td, J=8.1, 4.1 Hz, 1H), 2.06 (s, 6H), 1.20-1.11 (m, 4H). (Sulfonamide —NH not visible)

### Example 89: Preparation of Compound I-196

### Step 1: Cyclopentyl(triphenyl)phosphonium bromide

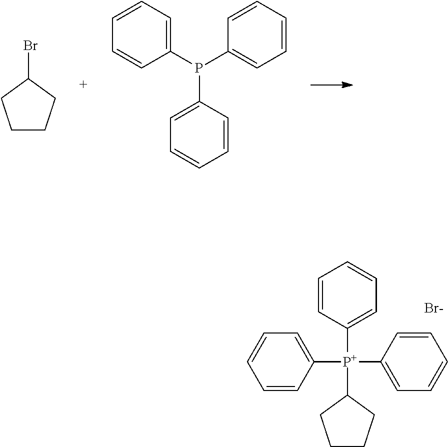 [SMILES: BrC1CCCC1 ; c1ccc(P(c2ccccc2)c2ccccc2)cc1 ; c1ccc([P+](c2ccccc2)(c2ccccc2)C2CCCC2)cc1 ; [Br-]]

A sealed pressure tube was charged with triphenylphosphane (150.20 g, 132.69 mL, 566.93 mmol) and bromocyclopentane (98.078 g, 72 mL, 644.95 mmol). The mixture was heated to and stirred at 140° C. for 21 h. To the resulting solid was added toluene (300 mL) and the heterogeneous mixture was stirred for 3 h before filtered through a Type “M” Glass filter by vacuum. The solids were rinsed with toluene (2×100 mL) and dried under vacuum. The product (194.60 g) was obtained as a white solid. cyclopentyl(triphenyl)phosphonium bromide (194.60 g, 83%) ¹H NMR (500 MHz, Chloroform-d) δ 7.96-7.89 (m, 5H), 7.81-7.62 (m, 10H), 5.65-5.51 (m, 1H), 2.66-2.52 (m, 2H), 1.94-1.83 (m, 2H), 1.68-1.53 (m, 2H), 1.31-1.21 (m, 2H).

### Step 2: 2-Bromo-1-(cyclopentylidenemethyl)-3-methyl-benzene

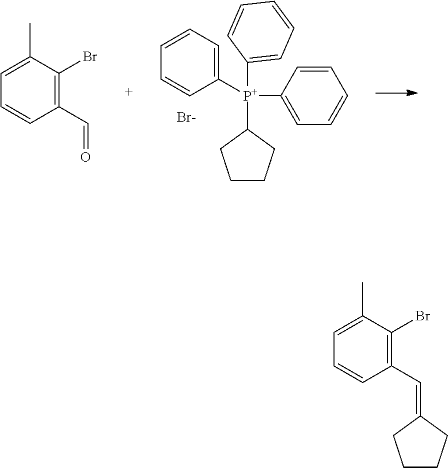

To a suspension of cyclopentyl(triphenyl)phosphonium bromide (195.93 g, 476.35 mmol) in anhydrous THE (340 mL) stirring at 0° C. under nitrogen was added dropwise a solution of potassium tert-butoxide in THE (550 mL of 1 M, 550.00 mmol). The mixture was stirred at 0° C. for 0.5 h. At the same temperature with stirring, the 2-bromo-3-methyl-benzaldehyde (47.87 g, 240.50 mmol) in anhydrous THE (340 mL) was added dropwise. The reaction was then stirred at rt for 18 h. The reaction mixture was cooled to 0° C. and then quenched with cold DI H₂O (1000 mL). The aqueous layer was extracted with EtOAc (3×250 mL). The combined organic layers were washed with aqueous saturated NaCl (250 mL), dried with anhydrous sodium sulfate, filtered, and concentrated under vacuum. The crude (192.44 g, light brown solid) was triturated with hexanes (1200 mL, 3 h) and filtered through a pad of silica gel (260 g) on a Type “M” Glass filter by vacuum. The solids were rinsed with hexanes (3×500 mL). The combined hexanes filtrate was concentrated under vacuum. The final product (56.86 g) was obtained as a colorless oil. 2-bromo-1-(cyclopentylidenemethyl)-3-methyl-benzene (56.86 g, 89%) ¹H NMR (500 MHz, Chloroform-d) δ 7.19-7.01 (m, 3H), 5.27 (s, 1H), 2.56-2.49 (m, 1H), 2.43 (s, 3H), 2.35-2.23 (m, 4H), 1.92-1.84 (m, 2H), 1.75-1.68 (m, 1H). ESI-MS m/z calc. 250.0357, expected mass not detected, Retention time: 3.92 minutes; LC method E.

### Step 3: 2-[2-(Cyclopentylidenemethyl)-6-methyl-phenyl]-4,4,5,5-tetramethyl-1,3,2-dioxaborolane

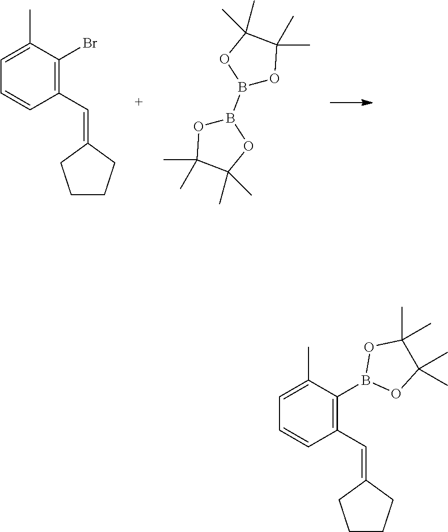

To a Parr Reactor was added 2-bromo-1-(cyclopentylidenemethyl)-3-methyl-benzene (17.65 g, 66.760 mmol), KOAc (16.48 g, 167.92 mmol) and anhydrous dioxane (350 mL). The mixture was degassed with Nitrogen for 0.5 h. Keeping the Nitrogen stream going, to the mixture was added 4,4,5,5-tetramethyl-2-(4,4,5,5-tetramethyl-1,3,2-dioxaborolan-2-yl)-1,3,2-dioxaborolane (30.63 g, 120.62 mmol) and Pd(dppf)Cl₂ (4.91 g, 6.7104 mmol). Degassing continued for an additional 15 min after addition. The tube was then capped quickly and tightly. The reaction mixture was heated to and stirred at 100° C. for 18 h. The reaction was quenched with aqueous saturated ammonium chloride (250 mL). The mixture was filtered through a pad of celite. The celite was rinsed with EtOAc (3×200 mL). The layers were separated. The aqueous layer was extracted with EtOAc (3×100 mL). The combined organic layers were washed with aqueous saturated NaCl (250 mL), dried over anhydrous sodium sulfate, filtered, and concentrated under vacuum. The crude was subjected to flash chromatography by (Pre-adsorbed to SiO₂) (330 g and then 220 g SiO₂, eluting 0 to 10% EtOAc/Hexanes in 75 min., Flow Rate=75 mL/min., Detection=UV254). Pure fractions were collected and concentrated under vacuum. The final product was obtained as a yellow-green oil. 2-[2-(cyclopentylidenemethyl)-6-methyl-phenyl]-4,4,5,5-tetramethyl-1,3,2-dioxaborolane (11.40 g, 54%) ¹H NMR (500 MHz, Chloroform-d) δ 7.21-6.91 (m, 3H), 5.17-5.09 (m, 1H), 2.42 (s, 3H), 2.39 (s, 1H), 2.31-2.20 (m, 4H), 1.90-1.77 (m, 2H), 1.75-1.59 (m, 1H), 1.41-1.31 (m, 12H). ESI-MS m/z calc. 298.21042, found 299.3 (M+1)⁺; Retention time: 3.78 minutes; LC method E.

### Step 4: tert-Butyl N-tert-butoxycarbonyl-N-[4-chloro-6-[2-(cyclopentylidenemethyl)-6-methyl-phenyl]-5-methyl-pyrimidin-2-yl]carbamate

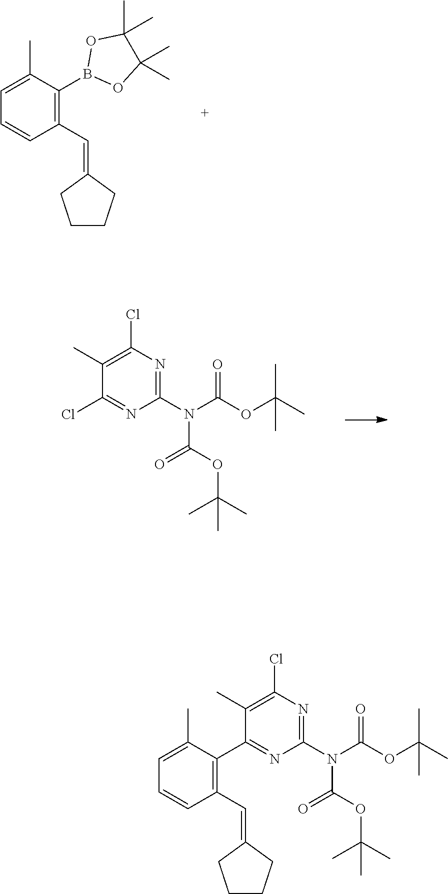 [SMILES: Cc1cccc(C=C2CCCC2)c1B1OC(C)(C)C(C)(C)O1 ; Cc1c(Cl)nc(N(C(=O)OC(C)(C)C)C(=O)OC(C)(C)C)nc1Cl ; Cc1cccc(C=C2CCCC2)c1-c1nc(N(C(=O)OC(C)(C)C)C(=O)OC(C)(C)C)nc(Cl)c1C]

2-[2-(Cyclopentylidenemethyl)-6-methyl-phenyl]-4,4,5,5-tetramethyl-1,3,2-dioxaborolane (11.40 g, 36.315 mmol), tert-butyl N-tert-butoxycarbonyl-N-(4,6-dichloro-5-methyl-pyrimidin-2-yl)carbamate (13.15 g, 33.027 mmol) and cesium carbonate (59.48 g, 182.56 mmol) were dissolved in 1,2-dimethoxyethane (180 mL) and water (180 mL). The solution was degassed with nitrogen for 30 min, then Pd(dppf)Cl₂ (2.73 g, 3.7310 mmol) was added to the reaction mixture with degassing continuing for an additional 15 min. The reaction mixture was heated to and stirred at 100° C. in an oil-bath for 3.5 h. The reaction mixture was quenched with DI H₂O (200 mL) and extracted with EtOAc (3×150 mL). The combined organic layers were washed with aqueous saturated NaCl (200 mL), dried over anhydrous sodium sulfate, filtered, and concentrated under vacuum. The crude was subjected to flash chromatography (Pre-adsorbed to SiO₂) (330 g SiO₂, eluting 0 to 10% EtOAc/Hexanes in 85 min., Flow Rate=55 mL/min., Detection=UV254). Appropriate fractions were collected and concentrated under vacuum. The final product (8.26 g) was obtained as a Yellow Oil. tert-butyl N-tert-butoxycarbonyl-N-[4-chloro-6-[2-(cyclopentylidenemethyl)-6-methyl-phenyl]-5-methyl-pyrimidin-2-yl]carbamate (8.26 g, 44%) ESI-MS m/z calc. 513.23944, found 514.4 (M+1)⁺; Retention time: 4.09 minutes; LC method E.

### Step 5: tert-butyl N-tert-butoxycarbonyl-N-[4-chloro-6-[2-(cyclopentylmethyl)-6-methyl-phenyl]-5-methyl-pyrimidin-2-yl]carbamate

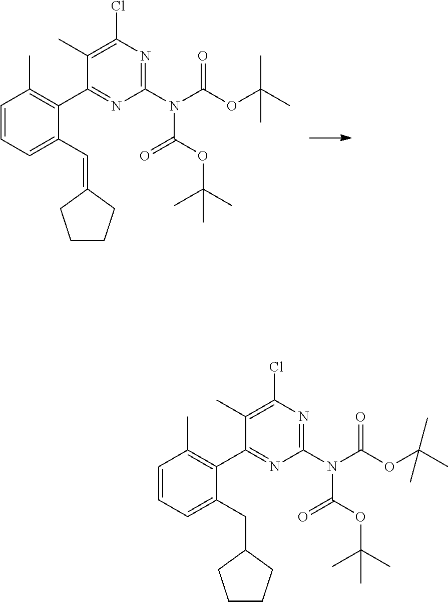 [SMILES: Cc1cccc(C=C2CCCC2)c1-c1nc(N(C(=O)OC(C)(C)C)C(=O)OC(C)(C)C)nc(Cl)c1C ; Cc1cccc(CC2CCCC2)c1-c1nc(N(C(=O)OC(C)(C)C)C(=O)OC(C)(C)C)nc(Cl)c1C]

To a solution of tert-butyl N-tert-butoxycarbonyl-N-[4-chloro-6-[2-(cyclopentylidenemethyl)-6-methyl-phenyl]-5-methyl-pyrimidin-2-yl]carbamate (1.446 g, 2.6723 mmol) in EtOAc (45 mL) was added Rhodium on alumina powder (557 mg, 0.2706 mmol). The reaction mixture was placed into a par shaker and shaken at 50 PSI of hydrogen for 3 hours. The mixture was then filtered through a pad of Celite and concentrated under reduced pressure to afford tert-butyl N-tert-butoxycarbonyl-N-[4-chloro-6-[2-(cyclopentylmethyl)-6-methyl-phenyl]-5-methyl-pyrimidin-2-yl]carbamate (1.453 g, 100%) as a colorless oil. ESI-MS m/z calc. 515.25507, found 516.4 (M+1)⁺; Retention time: 4.67 minutes; LC method E.

### Step 6: 4-Chloro-6-[2-(cyclopentylmethyl)-6-methyl-phenyl]-5-methyl-pyrimidin-2-amine

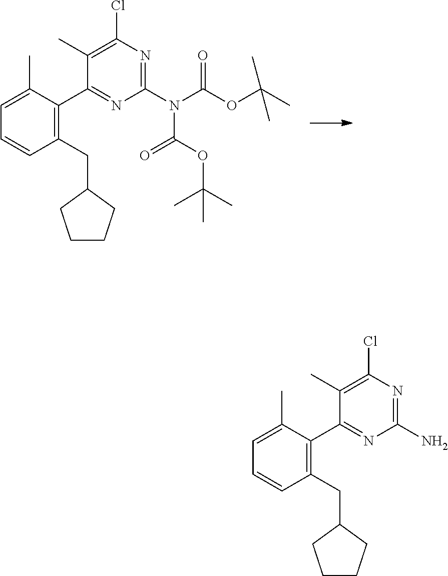 [SMILES: Cc1cccc(CC2CCCC2)c1-c1nc(N(C(=O)OC(C)(C)C)C(=O)OC(C)(C)C)nc(Cl)c1C ; Cc1cccc(CC2CCCC2)c1-c1nc(N)nc(Cl)c1C]

To a solution of tert-butyl N-tert-butoxycarbonyl-N-[4-chloro-6-[2-(cyclopentylmethyl)-6-methyl-phenyl]-5-methyl-pyrimidin-2-yl]carbamate (8.03 g, 14.782 mmol) in DCM (70 mL) was added HCl in dioxane (40 mL of 4 M, 160.00 mmol). The mixture was stirred at rt for 22 h. The reaction was quenched with saturated aqueous NaHCO₃ solution (50 mL), and the aqueous layer was extracted with DCM (3×30 mL), the combined DCM layers were washed with saturated aqueous NaCl (50 mL), dried over anhydrous sodium sulfate, filtered, and concentrated under vacuum to give 4-chloro-6-[2-(cyclopentylmethyl)-6-methyl-phenyl]-5-methyl-pyrimidin-2-amine (5.1719 g, 105%). ¹H NMR (500 MHz, DMSO-d6) δ 7.29-7.07 (m, 3H), 4.78 (s, 2H), 2.39-2.27 (m, 1H), 2.26-2.13 (m, 1H), 1.96 (s, 4H), 1.77 (s, 3H), 1.55-1.38 (m, 6H), 1.13-0.97 (m, 2H). ESI-MS m/z calc. 315.15024, found 316.0 (M+1)⁺; Retention time: 3.2 minutes; LC method E.

### Step 7: (5P)-4-Chloro-6-[2-(cyclopentylmethyl)-6-methylphenyl]-5-methylpyrimidin-2-amine, and (5M)-4-chloro-6-[2-(cyclopentylmethyl)-6-methylphenyl]-5-methylpyrimidin-2-amine

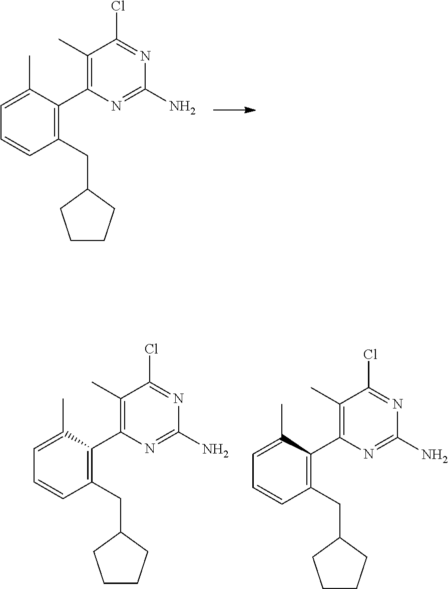

Racemic 4-chloro-6-[2-(cyclopentylmethyl)-6-methyl-phenyl]-5-methyl-pyrimidin-2-amine (12.8 g, 40.53 mmol) was subjected to SFC separation using the following conditions: IC (3×25 cm) column, eluent: 10% isopropanol/CO₂, 100 bar, 80 mL/min, 220 nm, inj. volume: 1 mL, 25 mg/mL in ethanol to give two atropisomers: IC-SFC peak 1: (5P)-4-chloro-6-[2-(cyclopentylmethyl)-6-methylphenyl]-5-methylpyrimidin-2-amine (6.1 g, 95%). ESI-MS m/z calc. 315.15024, found 316.22 (M+1)⁺; Retention time: 2.01 minutes, and IC-SFC peak 2: (5M)-4-chloro-6-[2-(cyclopentylmethyl)-6-methylphenyl]-5-methylpyrimidin-2-amine (6.4 g, 100%). ESI-MS m/z calc. 315.15024, found 316.22 (M+1)⁺; Retention time: 2.02 minutes. LC method A.

### Step 8: Methyl 3-[[4-chloro-6-[2-(cyclopentylmethyl)-6-methyl-phenyl]-5-methyl-pyrimidin-2-yl]sulfamoyl]benzoate (P atropisomer

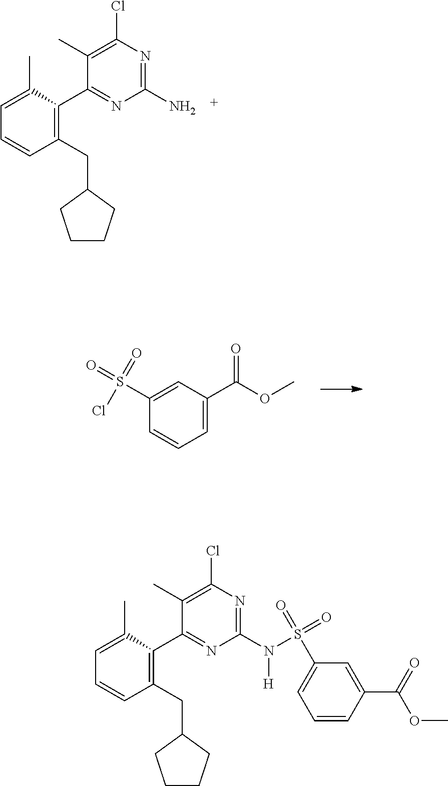

To a solution of 4-chloro-6-[2-(cyclopentylmethyl)-6-methyl-phenyl]-5-methyl-pyrimidin-2-amine (P atropisomer, 5.9 g, 18.680 mmol) and methyl 3-chlorosulfonylbenzoate (13.150 g, 56.040 mmol) in THF (120 mL) was added LiHMDS in THE (75 mL of 1 M, 75.000 mmol) dropwise over twenty minutes at −78° C. The reaction mixture was stirred for 2 h and then quenched with 1M hydrochloric acid (100 mL). The layers were separated, and the aqueous layer was extracted with ethyl acetate (2×250 mL). The combined organic layer was washed with brine, dried over sodium sulfate, filtered, and concentrated in vacuo. The resulting yellow oil was purified by flash chromatography (SiO₂, 120 g, loaded in EtOAc, eluted with 10% ethyl acetate in hexanes). The desired product fractions were combined and concentrated in vacuo to give methyl 3-[[4-chloro-6-[2-(cyclopentylmethyl)-6-methyl-phenyl]-5-methyl-pyrimidin-2-yl]sulfamoyl]benzoate (P atropisomer, 8.64 g, 89%) as a yellow sticky solid. ESI-MS m/z calc. 513.1489, found 514.7 (M+1)⁺; Retention time: 6.63 minutes; LC method D.

### Step 9: 3-[[4-Chloro-6-[2-(cyclopentylmethyl)-6-methyl-phenyl]-5-methyl-pyrimidin-2-yl]sulfamoyl]benzoic acid (P atropisomer

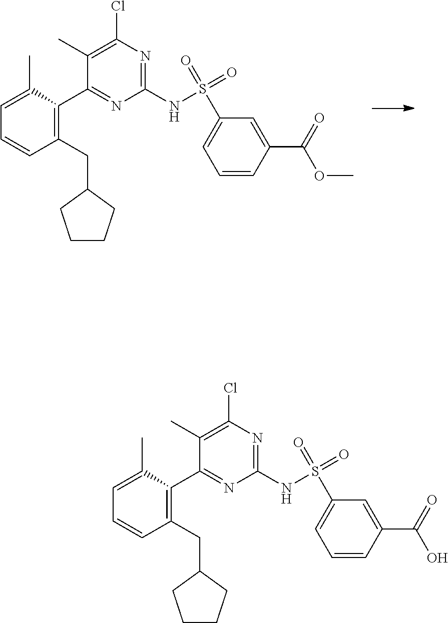

To a stirred solution of methyl 3-[[4-chloro-6-[2-(cyclopentylmethyl)-6-methyl-phenyl]-5-methyl-pyrimidin-2-yl]sulfamoyl]benzoate (P atropisomer, 8.64 g, 16.808 mmol) in THE (100 mL) was added aqueous NaOH (100 mL of 1 M, 100.00 mmol) and the reaction was stirred at rt for 2 h. The reaction mixture was extracted with ethyl acetate (2×250 mL) to remove impurities. The aqueous layer was acidified to pH=1 with 1M hydrochloric acid and extracted with ethyl acetate (2×250 mL). The combined organic layer was washed with brine, dried over sodium sulfate, filtered and concentrated in vacuo to give 3-[[4-chloro-6-[2-(cyclopentylmethyl)-6-methyl-phenyl]-5-methyl-pyrimidin-2-yl]sulfamoyl]benzoic acid (P atropisomer, 8.1 g, 95%) as a white solid. ESI-MS m/z calc. 499.13324, found 500.6 (M+1)⁺; Retention time: 5.81 minutes; LC method D.

### Step 10: 3-[[4-[(2R)-2-Amino-4,4-dimethyl-pentoxy]-6-[2-(cyclopentylmethyl)-6-methyl-phenyl]-5-methyl-pyrimidin-2-yl]sulfamoyl]benzoic acid (P atropisomer

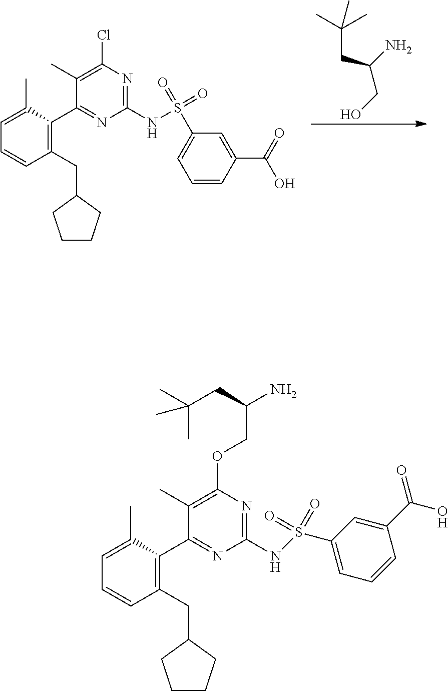

A solution of 3-[[4-chloro-6-[2-(cyclopentylmethyl)-6-methyl-phenyl]-5-methyl-pyrimidin-2-yl]sulfamoyl]benzoic acid (P atropisomer, 7 g, 13.860 mmol), (2R)-2-amino-4,4-dimethyl-pentan-1-ol (hydrochloride salt) (2.8 g, 16.699 mmol) and sodium t-butoxide (6 g, 62.433 mmol) in THE (140.00 mL) was stirred at rt for 2 h. More (2R)-2-amino-4,4-dimethyl-pentan-1-ol (hydrochloride salt) (1 g, 5.9639 mmol) and sodium t-butoxide (3 g, 31.216 mmol) was added and the reaction was stirred for another hour. The reaction mixture was quenched with 1 M HCl (200 mL) and extracted with ethyl acetate (3×500 mL). The combined organic layer was washed with brine, dried over sodium sulfate, filtered, and concentrated in vacuo. The resulting pale orange sticky solid was triturated with 10% ethyl acetate in hexanes and filtered. The cake was washed with hexanes and dried in vacuo to give 3-[[4-[(2R)-2-amino-4,4-dimethyl-pentoxy]-6-[2-(cyclopentylmethyl)-6-methyl-phenyl]-5-methyl-pyrimidin-2-yl]sulfamoyl]benzoic acid (hydrochloride salt) (P atropisomer, 7.0 g, 60%) as a pale yellow solid. ¹H NMR (500 MHz, DMSO-d6) δ 13.14 (s, 1H), 8.44-8.40 (m, 1H), 8.25-8.17 (m, 3H), 8.15-8.06 (m, 2H), 7.69-7.63 (m, 1H), 7.31-7.25 (m, 1H), 7.19-7.09 (m, 2H), 4.27 (d, J=12.0 Hz, 1H), 4.12 (dd, J=12.0, 6.1 Hz, 1H), 3.61-3.53 (m, 1H), 2.25-2.08 (m, 2H), 1.94-1.83 (m, 5H), 1.66 (s, 3H), 1.54 (dd, J=15.1, 4.3 Hz, 2H), 1.50-1.33 (m, 7H), 0.92 (s, 9H). ESI-MS m/z calc. 594.2876, found 595.6 (M+1)⁺; Retention time: 2.2 minutes; LC method H.

### Step 11: 3-{[(3P)-4-[2-(Cyclopentylmethyl)-6-methylphenyl]-6-{[(2R)-4,4-dimethyl-2-({[5-methyl-6-(propan-2-yl)-5H-pyrrolo[2,3-b]pyrazin-3-yl]methyl}amino)pentyl]oxy}-5-methylpyrimidin-2-yl]sulfamoyl}benzoic acid

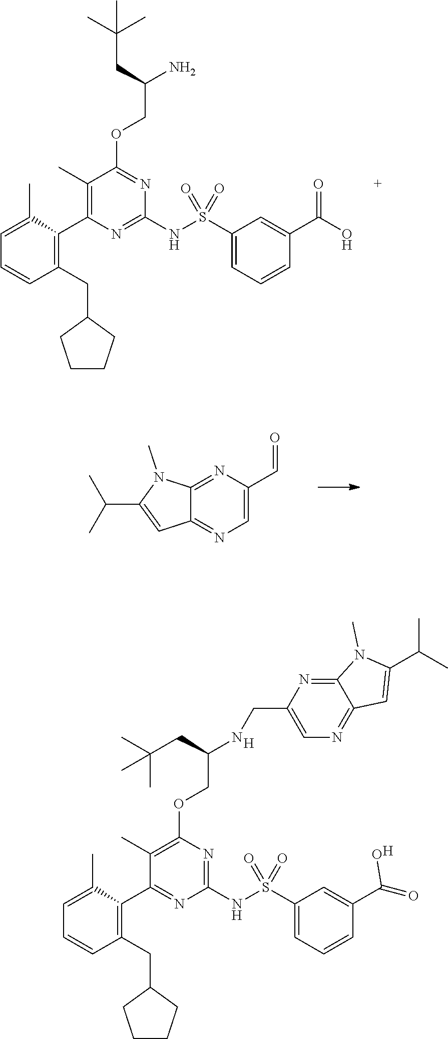

A 4 mL vial was charged under nitrogen with 3-{[(5P)-4-{[(2R)-2-amino-4,4-dimethylpentyl]oxy}-6-[2-(cyclopentylmethyl)-6-methylphenyl]-5-methylpyrimidin-2-yl]sulfamoyl}benzoic acid (hydrochloride salt) (59.1 mg, 0.09363 mmol), 6-isopropyl-5-methyl-pyrrolo[2,3-b]pyrazine-3-carbaldehyde (19.7 mg, 0.09693 mmol), anhydrous DCM (0.5 mL), and acetic acid (11.0 μL, 0.1934 mmol). The mixture was cooled down in an ice bath. DIEA (49.8 μL, 0.2859 mmol) was added, followed by sodium triacetoxyborohydride (106.0 mg, 0.5001 mmol), and the reaction was vigorously stirred at 0° C. for 5 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-{[(3P)-4-[2-(cyclopentylmethyl)-6-methylphenyl]-6-{[(2R)-4,4-dimethyl-2-({[5-methyl-6-(propan-2-yl)-5H-pyrrolo[2,3-b]pyrazin-3-yl]methyl}amino)pentyl]oxy}-5-methylpyrimidin-2-yl]sulfamoyl}benzoic acid (hydrochloride salt) (42.3 mg, 54%) as a yellow solid. ESI-MS m/z calc. 781.39856, found 782.2 (M+1)⁺; Retention time: 1.67 minutes. LC method A.

### Step 12: (5P,11R)-6-[2-(Cyclopentylmethyl)-6-methylphenyl]-11-(2,2-dimethylpropyl)-7-methyl-12-{[5-methyl-6-(propan-2-yl)-5H-pyrrolo[2,3-b]pyrazin-3-yl]methyl}-9-oxa-226-thia-3,5,12,19-tetraazatricyclo[12.3.1.1∧{4,8}]nonadeca-1(17),4(19),5,7,14(18),15-hexaene-2,2,13-trione (Compound I-196

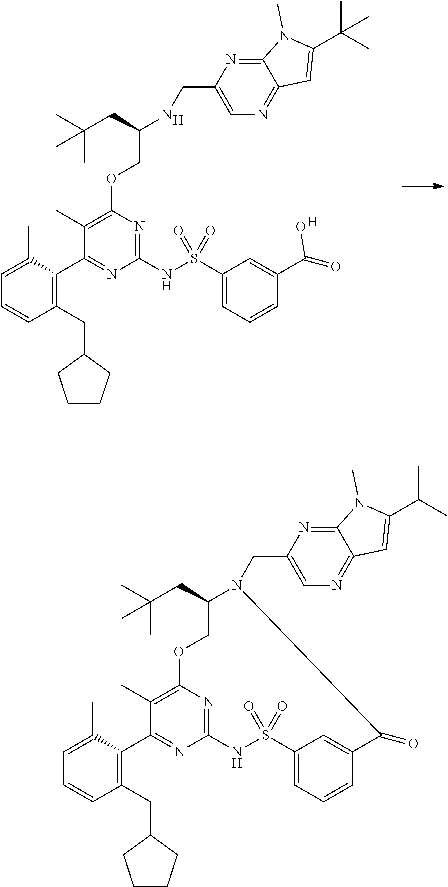

3-{[(3P)-4-[2-(Cyclopentylmethyl)-6-methylphenyl]-6-{[(2R)-4,4-dimethyl-2-({[5-methyl-6-(propan-2-yl)-5H-pyrrolo[2,3-b]pyrazin-3-yl]methyl}amino)pentyl]oxy}-5-methylpyrimidin-2-yl]sulfamoyl}benzoic acid (hydrochloride salt) (42.3 mg, 0.05065 mmol) was combined with CDMT (31.2 mg, 0.1777 mmol) in DMF (5.2 mL) and cooled to 0° C. N-methylmorpholine (33.5 μL, 0.3047 mmol) was added by syringe and the reaction was stirred at 0° C. for 30 minutes. The ice bath was then removed and stirring was continued for an additional 20 hours at room temperature. The reaction mixture was then partitioned between 50 mL 1M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (5P,11R)-6-[2-(cyclopentylmethyl)-6-methylphenyl]-11-(2,2-dimethylpropyl)-7-methyl-12-{[5-methyl-6-(propan-2-yl)-5H-pyrrolo[2,3-b]pyrazin-3-yl]methyl}-9-oxa-2λ⁶-thia-3,5,12,19-tetraazatricyclo[12.3.1.1∧{4,8}]nonadeca-1(17),4(19),5,7,14(18),15-hexaene-2,2,13-trione (17.7 mg, 45%) as a white solid. ¹H NMR (400 MHz, CDCl₃) δ 9.06 (t, J=1.8 Hz, 1H), 8.49 (s, 1H), 8.10 (dt, J=7.9, 1.1 Hz, 1H), 7.87 (dt, J=7.8, 1.3 Hz, 1H), 7.65 (t, J=7.8 Hz, 1H), 7.25-7.21 (m, 1H), 7.16 (d, J=7.6 Hz, 1H), 7.06 (d, J=7.5 Hz, 1H), 6.54 (s, 1H), 5.62 (dd, J=11.0, 4.2 Hz, 1H), 5.44 (d, J=15.8 Hz, 1H), 4.43 (d, J=15.8 Hz, 1H), 4.29-4.20 (m, 1H), 4.09 (t, J=11.3 Hz, 1H), 3.94 (s, 3H), 3.17 (hept, J=6.8 Hz, 1H), 2.36-2.28 (m, 2H), 2.05-1.93 (m, 2H), 1.85-1.75 (m, 5H), 1.72 (s, 3H), 1.52-1.44 (m, 3H), 1.40 (d, J=6.8 Hz, 3H), 1.37 (d, J=6.9 Hz, 4H), 1.13-0.95 (m, 3H), 0.65 (s, 9H). ESI-MS m/z calc. 763.388, found 764.4 (M+1)⁺; Retention time: 2.3 minutes. LC method A.

### Example 90: Preparation of Compound I-197

### Step 1: 6-Chloro-5-fluoro-2-iodo-pyridin-3-ol

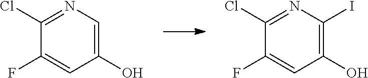 [SMILES: Oc1cnc(Cl)c(F)c1 ; Oc1cc(F)c(Cl)nc1I]

To a flask (2 L) were added 6-chloro-5-fluoro-pyridin-3-ol (29.15 g, 197.58 mmol), sodium carbonate (42.8 g, 403.82 mmol), and water (1040 mL). To the stirred mixture was added iodine (52.3 g, 206.06 mmol). The mixture was stirred at room temperature for 2.5 hours. Then, the pH of the solution was adjusted to 2 by HCl aqueous solution (1 N) and diluted with EtOAc (1 L+600 mL). The resulted solution was stirred for 10 min. The layers were separated, and the organic layer was washed with sat. Na₂S₂O₃ aqueous solution (500 mL) and brine (300 mL) respectively. The organic layer was dried over Na₂SO₄, filtered, and concentrated under reduced pressure to afford 6-chloro-5-fluoro-2-iodo-pyridin-3-ol (54.48 g, 101%) as a white solid. ¹H NMR (400 MHz, CDCl₃) δ 7.12 (d, J=8.1 Hz, 1H), 5.67-5.33 (br. s, 1H). ESI-MS m/z calc. 272.88538, found 274.0 (M+1)⁺; Retention time: 1.67 minutes; LC method I.

### Step 2: 5-Chloro-2-cyclopropyl-6-fluoro-furo[3,2-b]pyridine

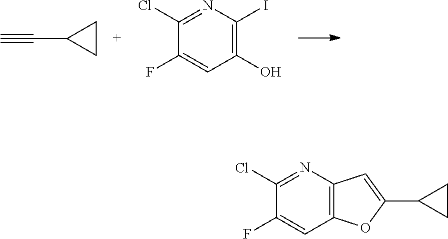

To a flask (25 mL) containing 6-chloro-5-fluoro-2-iodo-pyridin-3-ol (5.6 g, 20.460 mmol) was added DMF (56 mL). The resulting solution was degassed with N2 for 10 minutes. Then, to the solution were added bis(triphenylphosphine)palladium(II)chloride (190 mg, 0.2707 mmol), copper iodine (585 mg, 3.0717 mmol) and TEA (5.1546 g, 7.1 mL, 50.940 mmol). Then, the mixture was stirred at 60° C. for 10 minutes. After that, ethynyl cyclopropane (1.6380 g, 2.1 mL, 24.780 mmol) in DMF (10 mL) was added dropwise over 30 minutes. The mixture was stirred at 60° C. overnight. The mixture was diluted with EtOAc (100 mL) and filtered with Celite. The filtrate was diluted with water (300 mL) and extracted with Et₂O (150 mL×3). The combined organic phases were washed with brine (150 mL), dried with Na₂SO₄, filtered, and concentrated under reduced pressure to afford a crude product. The crude was purified with chromatography on silica gel (column: 40 g HP Gold; gradient: 2% ethyl acetate in heptane, 5 CV; 5% ethyl acetate in heptane, 3 CV; 10% ethyl acetate in heptane, 2 CV) to give 5-chloro-2-cyclopropyl-6-fluoro-furo[3,2-b]pyridine (3.7 g, 82%) as a white solid. ¹H NMR (400 MHz, CDCl₃) δ 7.47 (dd, J=7.9, 0.7 Hz, 1H), 6.51 (s, 1H), 2.11-2.04 (m, 1H), 1.13-1.01 (m, 4H). ESI-MS m/z calc. 211.02002, found 212.2 (M+1)⁺; Retention time: 1.88 minutes; LC method I.

### Step 3: Methyl 2-cyclopropyl-6-fluoro-furo[3,2-b]pyridine-5-carboxylate

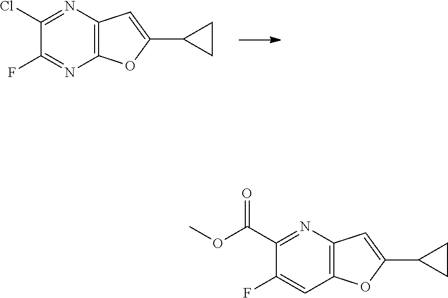 [SMILES: Fc1nc2oc(C3CC3)cc2nc1Cl ; COC(=O)c1nc2cc(C3CC3)oc2cc1F]

To a high pressure vial (35 mL, 150 psi, 120° C.) were added 5-chloro-2-cyclopropyl-6-fluoro-furo[3,2-b]pyridine (290 mg, 1.3704 mmol), bis(acetonitrile)dichloropalladium (7 mg, 0.0270 mmol), BINAP (17 mg, 0.0273 mmol) and TEA (210.54 mg, 0.29 mL, 2.0806 mmol), and MeOH (8 mL) was added. The vial was purged with carbon monoxide (CO) three times and then was filled with CO to 50 psi. The reaction mixture was heated in an oil bath at 100° C. overnight. After cooling the reaction mixture, excess CO was vented. Solvent was removed under reduced pressure and the crude product was purified by chromatography on a silica gel ((column: 12 g HP Gold; gradient: 5% ethyl acetate in heptane, 4 CV; 13% ethyl acetate in heptane, 18 CV) to afford methyl 2-cyclopropyl-6-fluoro-furo[3,2-b]pyridine-5-carboxylate (255.8 mg, 77%) as a yellowish solid. ¹H NMR (400 MHz, CDCl₃) δ 7.47 (dd, J=10.1, 0.9 Hz, 1H), 6.66 (s, 1H), 4.03 (s, 3H), 2.15-2.06 (m, 1H), 1.14-1.06 (m, 4H). ESI-MS m/z calc. 235.06447, found 236.2 (M+1)⁺; Retention time: 1.67 minutes; LC method I.

### Step 4: 2-Cyclopropyl-6-fluoro-furo[3,2-b]pyridine-5-carbaldehyde

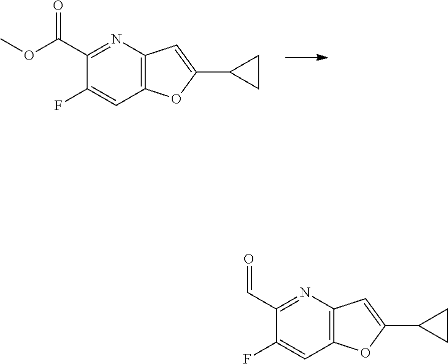 [SMILES: COC(=O)c1nc2cc(C3CC3)oc2cc1F ; O=Cc1nc2cc(C3CC3)oc2cc1F]

To a flask (25 mL) containing methyl 2-cyclopropyl-6-fluoro-furo[3,2-b]pyridine-5-carboxylate (250 mg, 1.0629 mmol) was added THF (5 mL). The solution was stirred at −78° C. for 15 minutes, then DIBAL-H in methylene chloride solution (3 mL of 1 M, 3.0000 mmol) was added dropwise over 5 minutes. The mixture was stirred at −78° C. for 45 minutes and quenched with MeOH (2 mL) and sat. NH₄Cl (2 mL). The solvents were concentrated under reduced pressure and the residue was diluted with sat. NH₄Cl (15 mL). The aqueous layer was extracted with EtOAc (10 mL×3). The combined organic layers were washed with brine (15 mL), dried over Na₂SO₄, filtered, and concentrated under reduced pressure to provide the crude product. The crude was purified by chromatography on silica gel (column: 12 g HP Gold; 5% ethyl acetate in heptane, 5 CV; 5-12%, 3 CV, 12% ethyl acetate in heptane 8 CV) to afford 2-cyclopropyl-6-fluoro-furo[3,2-b]pyridine-5-carbaldehyde (122 mg, 55%) as a white solid. ¹H NMR (400 MHz, CDCl₃) δ 10.30 (s, 1H), 7.48 (d, J=9.9 Hz, 1H), 6.67 (s, 1H), 2.17-2.08 (m, 1H), 1.18-1.07 (m, 4H). ESI-MS m/z calc. 205.05391, found 206.1 (M+1)⁺; Retention time: 2.96 minutes; LC method K.

### Step 5: 3-[[4-[(2R)-2-[(2-Cyclopropyl-6-fluoro-furo[3,2-b]pyridin-5-yl)methylamino]-3-(1-methylcyclopropyl)propoxy]-6-(2,6-dimethylphenyl)pyrimidin-2-yl]sulfamoyl]benzoic acid

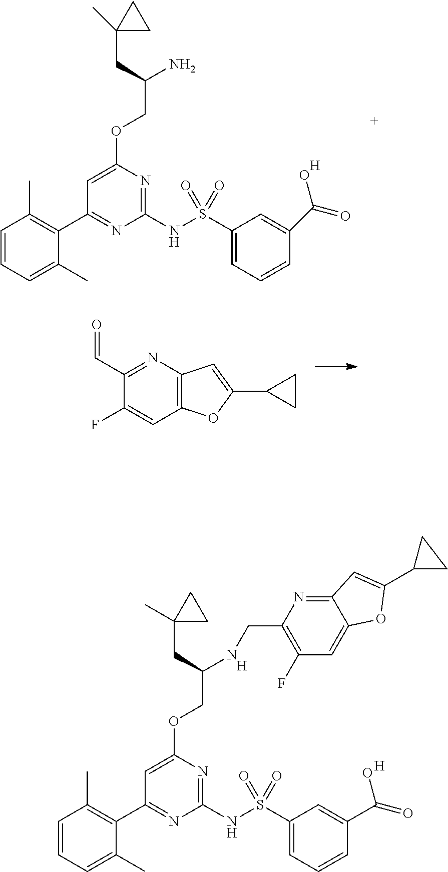

3-[[4-[(2R)-2-Amino-3-(1-methylcyclopropyl)propoxy]-6-(2,6-dimethylphenyl)pyrimidin-2-yl]sulfamoyl]benzoic acid (hydrochloride salt) (50 mg, 0.09140 mmol) was combined with 2-cyclopropyl-6-fluoro-furo[3,2-b]pyridine-5-carbaldehyde (21 mg, 0.1023 mmol) in DCM (0.5 mL) and acetic acid (10 μL, 0.1758 mmol) was added. The reaction mixture was cooled to 0° C. in an ice bath. DIEA (40 μL, 0.2296 mmol) was added followed by sodium triacetoxyborohydride (97 mg, 0.4577 mmol). After 1.5 hours at 0° C. the reaction mixture was quenched with 0.2 mL 3M HCl then diluted with methanol and DMSO until the reaction mixture became homogeneous. It was filtered and purified by preparative HPLC (1-99% ACN in water, HCl modifier, 15 minute run) to give 3-[[4-[(2R)-2-[(2-cyclopropyl-6-fluoro-furo[3,2-b]pyridin-5-yl)methylamino]-3-(1-methylcyclopropyl)propoxy]-6-(2,6-dimethylphenyl)pyrimidin-2-yl]sulfamoyl]benzoic acid (hydrochloride salt) (49.4 mg, 73%). ESI-MS m/z calc. 699.2527, found 700.7 (M+1)⁺; Retention time: 0.59 minutes; LC method B.

### Step 6: (11R)-12-[(2-Cyclopropyl-6-fluoro-furo[3,2-b]pyridin-5-yl)methyl]-6-(2,6-dimethylphenyl)-11-[(I-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-197

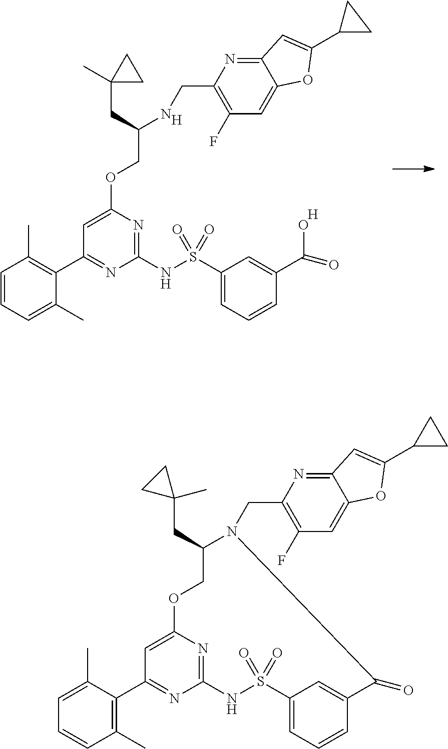

3-[[4-[(2R)-2-[(2-Cyclopropyl-6-fluoro-furo[3,2-b]pyridin-5-yl)methylamino]-3-(1-methylcyclopropyl)propoxy]-6-(2,6-dimethylphenyl)pyrimidin-2-yl]sulfamoyl]benzoic acid (hydrochloride salt) (49.4 mg, 0.06710 mmol) was combined with CDMT (14 mg, 0.07974 mmol) in DMF (3.5 mL). The reaction mixture was cooled to 0° C. in an ice bath and N-methylmorpholine (50 μL, 0.4548 mmol) was added. After 20 minutes the reaction mixture was removed from the ice bath allowed to warm to room temperature and stirred for 16 hours. Volatiles were removed by rotary evaporation under reduced pressure and the resulting residue was dissolved in DMSO/methanol, filtered, and purified by preparative HPLC (1-99% ACN in water, HCl modifier, 15 minute run) to give (11R)-12-[(2-cyclopropyl-6-fluoro-furo[3,2-b]pyridin-5-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (29.2 mg, 62%). ESI-MS m/z calc. 681.2421, found 682.8 (M+1)⁺; Retention time: 2.07 minutes; LC method A. ¹H NMR (400 MHz, Chloroform-d) δ 9.14 (t, J=1.8 Hz, 1H), 8.08 (d, J=7.9 Hz, 1H), 7.86 (d, J=7.6 Hz, 1H), 7.64 (t, J=7.8 Hz, 1H), 7.37 (d, J=9.6 Hz, 1H), 7.20 (t, J=7.6 Hz, 1H), 7.06 (d, J=7.6 Hz, 2H), 6.63 (s, 1H), 6.21 (s, 1H), 5.58 (dd, J=11.4, 4.2 Hz, 1H), 5.47 (d, J=16.2 Hz, 1H), 4.33 (t, J=11.1 Hz, 1H), 4.16 (d, J=16.2 Hz, 1H), 3.81 (t, J=11.5 Hz, 1H), 2.11-2.04 (m, 1H), 2.02 (s, 6H), 1.80 (d, J=14.9 Hz, 1H), 1.45 (dd, J=15.0, 10.2 Hz, 1H), 1.09-0.96 (m, 4H), 0.55 (s, 3H), 0.41 (dt, J=10.1, 5.3 Hz, 1H), 0.30-0.23 (m, 1H), 0.18-0.11 (m, 1H), 0.06-0.02 (m, 1H). Sulfonamide —NH not visible

### Example 91: Preparation of Compound III-25

### Step 1: 1-Isopropylpyrrolo[2,3-c]pyridine-5-carbonitrile

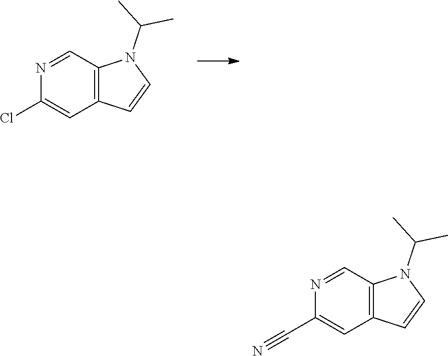

5-Chloro-1-isopropyl-pyrrolo[2,3-c]pyridine (195 mg, 1.002 mmol) was dissolved in DMF (2 mL), and the reaction vial was purged with nitrogen. Dicyanozinc (70 mg, 0.5960 mmol), zinc (6 mg, 0.09173 mmol) (dust), and Pd(dppf)Cl₂ (29 mg, 0.03963 mmol) were added. The vial was flushed with nitrogen, fitted with an unpierced cap, and heated at 130° C. for 1 hour then 160° C. for 3 hours. The reaction mixture was cooled to room temperature and partitioned between 30 mL water and 30 mL ethyl acetate. The layers were separated and the aqueous was extracted 2×20 mL ethyl acetate. The combined organics were washed with 20 mL each of water and brine then dried over sodium sulfate. The resulting crude material was purified by chromatography on silica gel eluting with 0-100% ethyl acetate in hexanes gradient to give 1-isopropylpyrrolo[2,3-c]pyridine-5-carbonitrile (138 mg, 74%) ESI-MS m/z calc. 185.09529, found 186.3 (M+1)⁺; Retention time: 0.47 minutes as a colorless oil. LC method B.

### Step 2: 1-Isopropylpyrrolo[2,3-c]pyridine-5-carbaldehyde

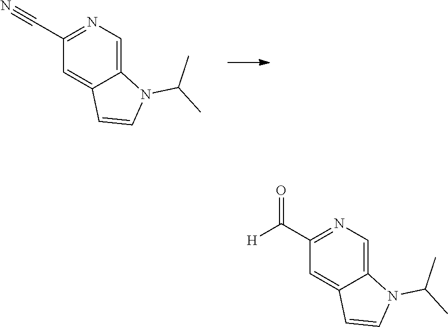

1-Isopropylpyrrolo[2,3-c]pyridine-5-carbonitrile (138 mg, 0.7450 mmol) was dissolved in anhydrous THE (5 mL) under a nitrogen atmosphere. The reaction was cooled to −78° C. in a dry ice/acetone bath, and diisobutylaluminum hydride (1.1 mL of 1 M, 1.100 mmol) as a solution in DCM was added dropwise. Stirring was continued with the reaction in the cooling bath for 1 hour 30 minutes. The reaction mixture was quenched with 20 mL of a saturated aqueous solution of Rochelle salts and diluted with DCM. The reaction mixture was removed from the cooling bath and allowed to warm to room temperature and stirred at this temperature for 16 h. The layers were separated, and the aqueous layer was extracted 2× with DCM. The combined organics were dried over sodium sulfate, filtered, and concentrated under reduced pressure. The crude material was purified by silica gel column chromatography using a gradient of 0-60% ethyl acetate in hexanes to provide 1-isopropylpyrrolo[2,3-c]pyridine-5-carbaldehyde (54.4 mg, 39%). ESI-MS m/z calc. 188.09496, found 189.2 (M+1)⁺; Retention time: 0.34 minutes. LC method B. ¹H NMR (400 MHz, Chloroform-d) δ 10.19 (s, 1H), 8.93 (d, J=1.0 Hz, 1H), 8.31 (d, J=1.0 Hz, 1H), 7.49 (d, J=3.2 Hz, 1H), 6.72 (d, J=3.2 Hz, 1H), 4.87 (hept, J=6.7 Hz, 1H), 1.63 (d, J=6.7 Hz, 6H).

### Step 3: 3-[[4-(2,6-Dimethylphenyl)-6-[(2R)-2-[(I-isopropylpyrrolo[2,3-c]pyridin-5-yl)methylamino]-3-(1-methylcyclopropyl)propoxy]pyrimidin-2-yl]sulfamoyl]benzoic acid

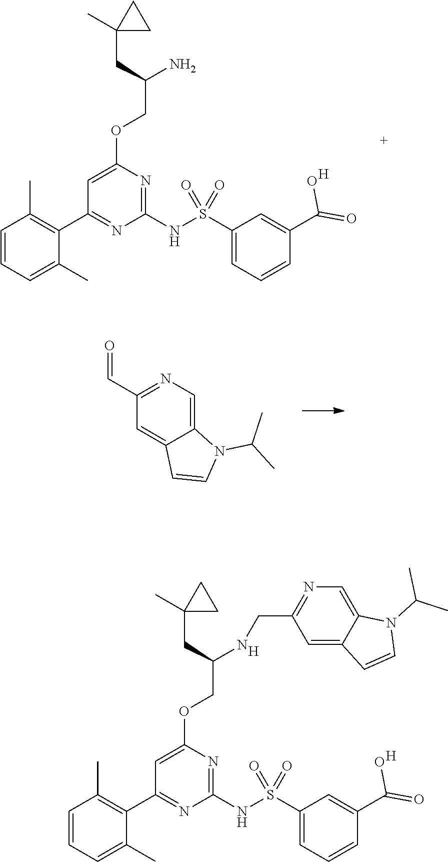

3-[[4-[(2R)-2-Amino-3-(1-methylcyclopropyl)propoxy]-6-(2,6-dimethylphenyl)pyrimidin-2-yl]sulfamoyl]benzoic acid (hydrochloride salt) (30 mg, 0.05484 mmol) was combined with 1-isopropylpyrrolo[2,3-c]pyridine-5-carbaldehyde (12 mg, 0.06375 mmol) in DCM (0.3 mL) and acetic acid (5 μL, 0.08792 mmol) was added. The reaction mixture was cooled to 0° C. in an ice bath. DIEA (25 μL, 0.1435 mmol) was added followed by sodium triacetoxyborohydride (60 mg, 0.2831 mmol). After 1.5 hours at 0° C. the reaction mixture was quenched with 0.2 mL 3M HCl then diluted with methanol and DMSO until the reaction mixture became homogeneous. It was filtered and purified by preparative HPLC (1-99% ACN in water, HCl modifier, 15 minute run) to give 3-[[4-(2,6-dimethylphenyl)-6-[(2R)-2-[(1-isopropylpyrrolo[2,3-c]pyridin-5-yl)methylamino]-3-(1-methylcyclopropyl)propoxy]pyrimidin-2-yl]sulfamoyl]benzoic acid (hydrochloride salt) (24.5 mg, 62%). ESI-MS m/z calc. 682.29376, found 683.9 (M+1)⁺; Retention time: 0.55 minutes; LC method B.

### Step 4: (11R)-6-(2,6-Dimethylphenyl)-12-[(I-isopropylpyrrolo[2,3-c]pyridin-5-yl)methyl]-11-[(I-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound III-25

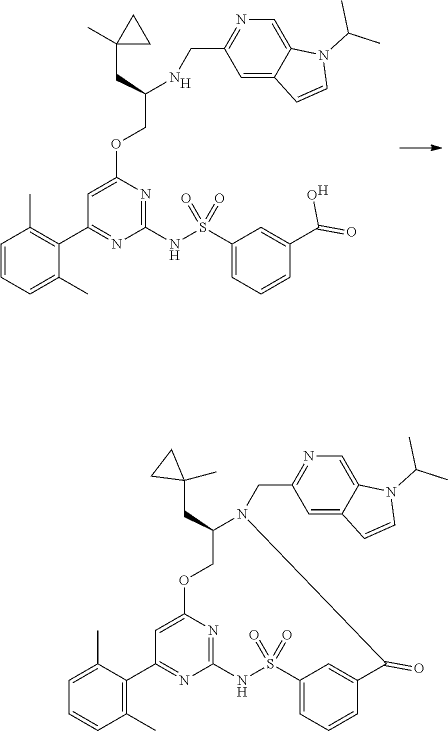

3-[[4-(2,6-Dimethylphenyl)-6-[(2R)-2-[(1-isopropylpyrrolo[2,3-c]pyridin-5-yl)methylamino]-3-(1-methylcyclopropyl)propoxy]pyrimidin-2-yl]sulfamoyl]benzoic acid (hydrochloride salt) (24.5 mg, 0.03406 mmol) was combined with CDMT (7.5 mg, 0.04272 mmol) in DMF (3.5 mL). The reaction mixture was cooled to 0° C. in an ice bath and N-methylmorpholine (30 μL, 0.2729 mmol) was added. After 20 minutes the reaction mixture was removed from the ice bath allowed to warm to room temperature and stirred for 16 hours. Volatiles were removed by rotary evaporation under reduced pressure and the resulting residue was dissolved in DMSO/methanol, filtered, and purified by preparative HPLC (1-99% ACN in water, HCl modifier, 15 minute run) to give (11R)-6-(2,6-dimethylphenyl)-12-[(1-isopropylpyrrolo[2,3-c]pyridin-5-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (12.2 mg, 50%). ESI-MS m/z calc. 664.2832, found 665.7 (M+1)⁺; Retention time: 1.36 minutes; LC method A. ¹H NMR (400 MHz, Chloroform-d) δ 8.83 (d, J=6.3 Hz, 1H), 8.57 (t, J=1.8 Hz, 1H), 8.28 (s, 1H), 8.13 (d, J=7.9 Hz, 1H), 7.95-7.89 (m, 2H), 7.69 (t, J=7.8 Hz, 1H), 7.21 (t, J=7.6 Hz, 1H), 7.06 (d, J=7.6 Hz, 2H), 6.85 (d, J=3.0 Hz, 1H), 6.22 (s, 1H), 5.64 (d, J=15.5 Hz, 1H), 5.02 (d, J=15.6 Hz, 1H), 4.91 (dd, J=10.3, 4.4 Hz, 1H), 4.86-4.73 (m, 2H), 4.47 (td, J=7.8, 4.0 Hz, 1H), 2.00 (s, 6H), 1.91-1.84 (m, 2H), 1.68 (d, J=6.7 Hz, 6H), 0.44 (s, 3H), 0.37-0.31 (m, 1H), 0.24-0.18 (m, 1H), 0.15-0.08 (m, 2H). (Sulfonamide NH and presumed HCl not visible)

### Example 92: Preparation of Compound I-202 and Compound I-203

### Step 1: 3-[[4-(2-Isopentyl-6-methyl-phenyl)-6-[(2R)-2-[(6-isopropyl-5-methyl-pyrrolo[2,3-b]pyrazin-3-yl)methylamino]-4,4-dimethyl-pentoxy]-5-methyl-pyrimidin-2-yl]sulfamoyl]benzoic acid

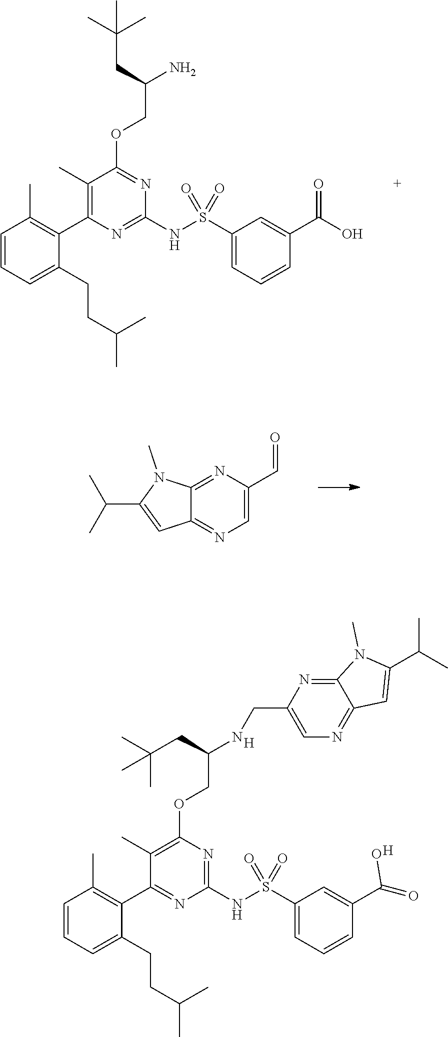

A 4 mL vial was charged under nitrogen with 3-[[4-[(2R)-2-amino-4,4-dimethyl-pentoxy]-6-(2-isopentyl-6-methyl-phenyl)-5-methyl-pyrimidin-2-yl]sulfamoyl]benzoic acid (80 mg, 0.1373 mmol), 6-isopropyl-5-methyl-pyrrolo[2,3-b]pyrazine-3-carbaldehyde (28.1 mg, 0.1383 mmol), anhydrous DCM (0.77 mL), and acetic acid (15.7 μL, 0.2761 mmol). The mixture was cooled down in an ice bath. DIEA (71.8 μL, 0.4122 mmol) was added, followed by sodium triacetoxyborohydride (146.5 mg, 0.6912 mmol), and the reaction was vigorously stirred at 0° C. for 4 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-(2-isopentyl-6-methyl-phenyl)-6-[(2R)-2-[(6-isopropyl-5-methyl-pyrrolo[2,3-b]pyrazin-3-yl)methylamino]-4,4-dimethyl-pentoxy]-5-methyl-pyrimidin-2-yl]sulfamoyl]benzoic acid (hydrochloride salt) (49.0 mg, 44%) as a yellow solid. ESI-MS m/z calc. 769.39856, found 770.5 (M+1)⁺; Retention time: 1.73 minutes. LC method A.

### Step 2: (5P,11R)-11-(2,2-dimethylpropyl)-7-methyl-6-[2-methyl-6-(3-methylbutyl)phenyl]-12-{[5-methyl-6-(propan-2-yl)-5H-pyrrolo[2,3-b]pyrazin-3-yl]methyl}-9-oxa-2λ⁶-thia-3,5,12,19-tetraazatricyclo[12.3.1.1∧{4,8}]nonadeca-1(17),4(19),5,7,14(18),15-hexaene-2,2,13-trione, Compound I-202, and (5M,11R)-11-(2,2-dimethylpropyl)-7-methyl-6-[2-methyl-6-(3-methylbutyl)phenyl]-12-{[5-methyl-6-(propan-2-yl)-5H-pyrrolo[2,3-b]pyrazin-3-yl]methyl}-9-oxa-2λ⁶-thia-3,5,12,19-tetraazatricyclo[12.3.1.1∧{4,8}]nonadeca-1(17),4(19),5,7,14(18),15-hexaene-2,2,13-trione, Compound I-203

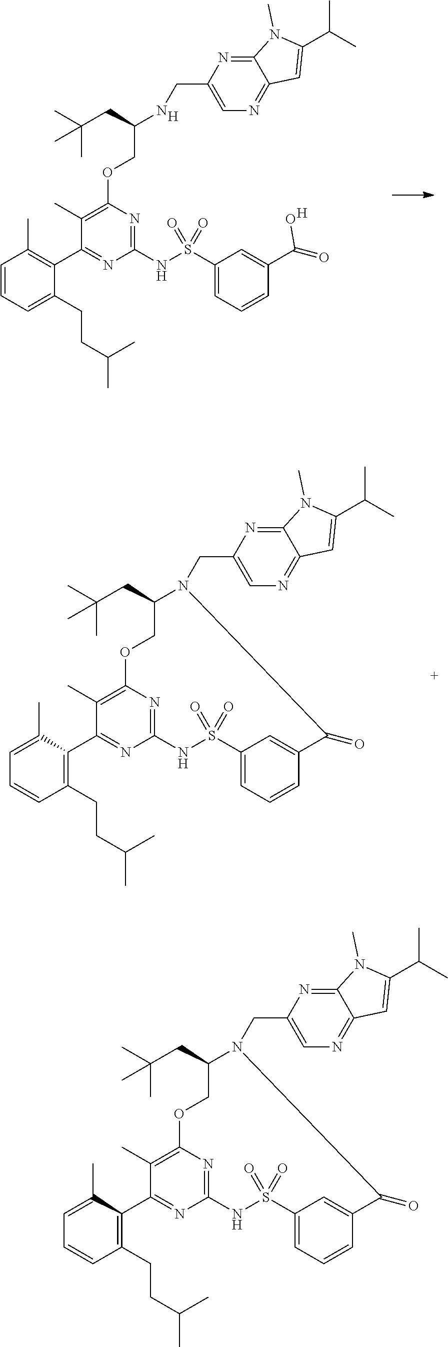

3-[[4-(2-Isopentyl-6-methyl-phenyl)-6-[(2R)-2-[(6-isopropyl-5-methyl-pyrrolo[2,3-b]pyrazin-3-yl)methylamino]-4,4-dimethyl-pentoxy]-5-methyl-pyrimidin-2-yl]sulfamoyl]benzoic acid (hydrochloride salt) (49.0 mg, 0.06076 mmol) was combined with CDMT (32.2 mg, 0.1834 mmol) in DMF (6.1 mL) and cooled to 0° C. N-methylmorpholine (40.1 μL, 0.3647 mmol) was added by syringe and the reaction was stirred at 0° C. for 30 minutes. The ice bath was then removed and stirring was continued for an additional 20 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give 28 mg of a white solid which was subjected to chiral SFC (ChiralPak AS (21.2×250 mm, 5 m column; 40° C., isocratic mode, 35% MeOH+20 mM NH₃, 70 mL/min, concentration 28 mg/mL (methanol), injection volume 500 μL, pressure 185 bar, wavelength 210 nm). For each separated isomer, the solvent was evaporated. The resulting solid was dissolved in DMSO (1 mL) and purified by reverse phase HPLC (1-99% acetonitrile/5 mM aqueous HCl over 25 min) to give as solids: SFC peak 1 (5P,11R)-11-(2,2-dimethylpropyl)-7-methyl-6-[2-methyl-6-(3-methylbutyl)phenyl]-12-{[5-methyl-6-(propan-2-yl)-5H-pyrrolo[2,3-b]pyrazin-3-yl]methyl}-9-oxa-2λ⁶-thia-3,5,12,19-tetraazatricyclo[12.3.1.1∧{4,8}]nonadeca-1(17),4(19),5,7,14(18),15-hexaene-2,2,13-trione (5.9 mg, 24%) ¹H NMR (400 MHz, CDCl₃) δ 9.06 (t, J=1.9 Hz, 1H), 8.48 (s, 1H), 8.10 (dt, J=8.0, 1.3 Hz, 1H), 7.88 (dt, J=7.7, 1.4 Hz, 1H), 7.65 (t, J=7.8 Hz, 1H), 7.25-7.21 (m, 1H), 7.13 (d, J=7.6 Hz, 1H), 7.05 (d, J=7.5 Hz, 1H), 6.50 (s, 1H), 5.62 (dd, J=11.0, 4.2 Hz, 1H), 5.43 (d, J=15.8 Hz, 1H), 4.42 (d, J=15.8 Hz, 1H), 4.29-4.20 (m, 1H), 4.10 (t, J=11.3 Hz, 1H), 3.92 (s, 3H), 3.16 (hept, J=6.9 Hz, 1H), 2.37-2.20 (m, 2H), 1.84-1.76 (m, 4H), 1.74 (s, 3H), 1.61 (dd, J=15.0, 1.6 Hz, 1H), 1.52-1.42 (m, 1H), 1.40 (d, J=6.8 Hz, 3H), 1.37 (d, J=6.8 Hz, 3H), 1.34-1.21 (m, 2H), 0.82 (d, J=6.4 Hz, 6H), 0.65 (s, 9H). ESI-MS m/z calc. 751.388, found 752.2 (M+1)⁺; Retention time: 2.06 minutes; and SFC Peak 2 (5M,11R)-11-(2,2-dimethylpropyl)-7-methyl-6-[2-methyl-6-(3-methylbutyl)phenyl]-12-{[5-methyl-6-(propan-2-yl)-5H-pyrrolo[2,3-b]pyrazin-3-yl]methyl}-9-oxa-2λ⁶-thia-3,5,12,19-tetraazatricyclo[12.3.1.1∧{4,8}]nonadeca-1(17),4(19),5,7,14(18),15-hexaene-2,2,13-trione (5.7 mg, 24%) ¹H NMR (400 MHz, CDCl₃) δ 9.04 (t, J=1.8 Hz, 1H), 8.49 (s, 1H), 8.06 (dt, J=8.0, 1.4 Hz, 1H), 7.85 (dt, J=7.7, 1.4 Hz, 1H), 7.63 (t, J=7.8 Hz, 1H), 7.25-7.21 (m, 1H), 7.13-7.05 (m, 2H), 6.55 (s, 1H), 5.65 (dd, J=10.9, 4.0 Hz, 1H), 5.41 (d, J=15.8 Hz, 1H), 4.44 (d, J=15.8 Hz, 1H), 4.23-4.13 (m, 1H), 4.06 (t, J=11.2 Hz, 1H), 3.95 (s, 3H), 3.17 (hept, J=6.8 Hz, 1H), 2.24-2.12 (m, 1H), 2.01 (s, 3H), 2.00-1.88 (m, 1H), 1.82 (dd, J=15.1, 9.3 Hz, 1H), 1.73 (s, 3H), 1.60 (d, J=14.8 Hz, 1H), 1.40 (d, J=6.8 Hz, 3H), 1.38 (d, J=6.8 Hz, 3H), 1.29-1.17 (m, 3H), 0.69 (s, 9H), 0.65 (d, J=6.1 Hz, 3H), 0.58 (d, J=6.1 Hz, 3H). ESI-MS m/z calc. 751.388, found 752.2 (M+1)⁺; Retention time: 2.01 minutes. LC method A.

### Example 93: Preparation of Compound I-206

### Step 1: 3-{[(]P)-4-{[(2R)-2-[({6-tert-butyl-5-methyl-5H-pyrrolo[2,3-b]pyrazin-3-yl}methyl)amino]-4,4-dimethylpentyl]oxy}-5-methyl-6-[2-methyl-6-(2-methylpropyl)phenyl]pyrimidin-2-yl]sulfamoyl}benzoic acid

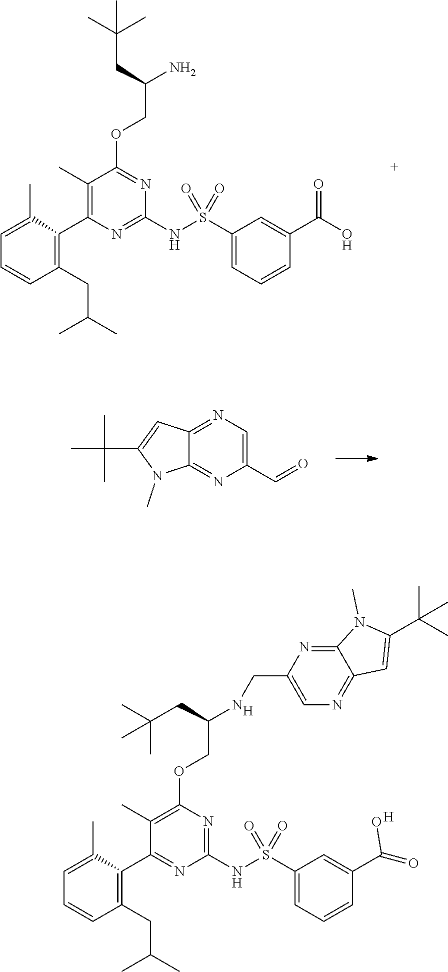

A 4 mL vial was charged under nitrogen with 3-{[(1P)-4-{[(2R)-2-amino-4,4-dimethylpentyl]oxy}-5-methyl-6-[2-methyl-6-(2-methylpropyl)phenyl]pyrimidin-2-yl]sulfamoyl}benzoic acid (hydrochloride salt) (66 mg, 0.1091 mmol), 6-tert-butyl-5-methyl-pyrrolo[2,3-b]pyrazine-3-carbaldehyde (24.0 mg, 0.1105 mmol), anhydrous DCM (0.6 mL), and acetic acid (12.5 μL, 0.2198 mmol). The mixture was cooled down in an ice bath. DIEA (57.1 μL, 0.3278 mmol) was added, followed by sodium triacetoxyborohydride (120.0 mg, 0.5662 mmol), and the reaction was vigorously stirred at 0° C. for 5 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-{[(1P)-4-{[(2R)-2-[({6-tert-butyl-5-methyl-5H-pyrrolo[2,3-b]pyrazin-3-yl}methyl)amino]-4,4-dimethylpentyl]oxy}-5-methyl-6-[2-methyl-6-(2-methylpropyl)phenyl]pyrimidin-2-yl]sulfamoyl}benzoic acid (hydrochloride salt) (50.0 mg, 55%) as a yellow solid. ESI-MS m/z calc. 769.39856, found 770.3 (M+1)⁺; Retention time: 1.64 minutes; LC method A.

### Step 2: (5P,1R)-12-({6-tert-Butyl-5-methyl-5H-pyrrolo[2,3-b]pyrazin-3-yl}methyl)-11-(2,2-dimethylpropyl)-7-methyl-6-[2-methyl-6-(2-methylpropyl)phenyl]-9-oxa-2λ⁶-thia-3,5,12,19-tetraazatricyclo[12.3.1.1∧{4,8}]nonadeca-1(17),4(19),5,7,14(18),15-hexaene-2,2,13-trione (Compound I-206

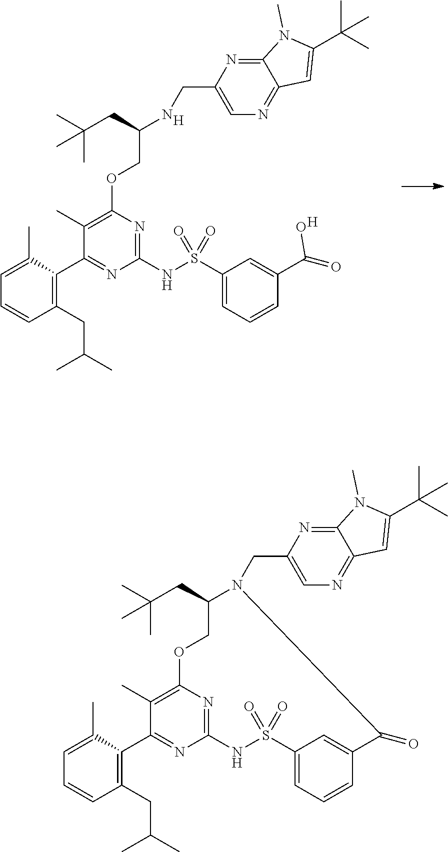

3-{[(1P)-4-{[(2R)-2-[({6-tert-Butyl-5-methyl-5H-pyrrolo[2,3-b]pyrazin-3-yl}methyl)amino]-4,4-dimethylpentyl]oxy}-5-methyl-6-[2-methyl-6-(2-methylpropyl)phenyl]pyrimidin-2-yl]sulfamoyl}benzoic acid (hydrochloride salt) (50 mg, 0.06014 mmol) was combined with CDMT (38.1 mg, 0.2170 mmol) in DMF (4.3 mL) and cooled to 0° C. N-methylmorpholine (40.0 μL, 0.3638 mmol) was added by syringe and the reaction was stirred at 0° C. for 30 minutes. The ice bath was then removed and stirring was continued for an additional 20 hours at room temperature. The reaction mixture was then partitioned between 50 mL 1M HCl and 50 mL ethyl acetate. The layers were separated, and the aqueous layer was extracted with an additional 50 mL ethyl acetate. The combined organic layers were washed 2×50 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (5P,11R)-12-({6-tert-butyl-5-methyl-5H-pyrrolo[2,3-b]pyrazin-3-yl}methyl)-11-(2,2-dimethylpropyl)-7-methyl-6-[2-methyl-6-(2-methylpropyl)phenyl]-9-oxa-2λ⁶-thia-3,5,12,19-tetraazatricyclo[12.3.1.1∧{4,8}]nonadeca-1(17),4(19),5,7,14(18),15-hexaene-2,2,13-trione (14.1 mg, 61%) as a yellow solid. ¹H NMR (400 MHz, CDCl₃) δ 9.08 (t, J=1.8 Hz, 1H), 8.49 (s, 1H), 8.10 (dt, J=8.0, 1.5 Hz, 1H), 7.87 (dt, J=7.7, 1.4 Hz, 1H), 7.64 (t, J=7.8 Hz, 1H), 7.26-7.21 (m, 1H), 7.12 (d, J=7.7 Hz, 1H), 7.06 (d, J=7.5 Hz, 1H), 6.53 (s, 1H), 5.64 (dd, J=11.0, 4.2 Hz, 1H), 5.44 (d, J=15.8 Hz, 1H), 4.43 (d, J=15.9 Hz, 1H), 4.30-4.21 (m, 1H), 4.13-4.05 (m, 4H), 2.29-2.11 (m, 2H), 1.84-1.74 (m, 5H), 1.71 (s, 3H), 1.61 (dd, J=15.2, 1.5 Hz, 1H), 1.50 (s, 9H), 0.81 (d, J=5.0 Hz, 3H), 0.79 (d, J=5.0 Hz, 3H), 0.65 (s, 9H). ESI-MS m/z calc. 751.388, found 752.6 (M+1)⁺; Retention time: 2.06 minutes. LC method A.

### Example 94: Preparation of Compound I-210

### Step 1: 2-Chloro-6-isopropyl-7-nitro-furo[2,3-b]pyrazine and 2-chloro-6-isopropyl-3-nitro-furo[2,3-b]pyrazine

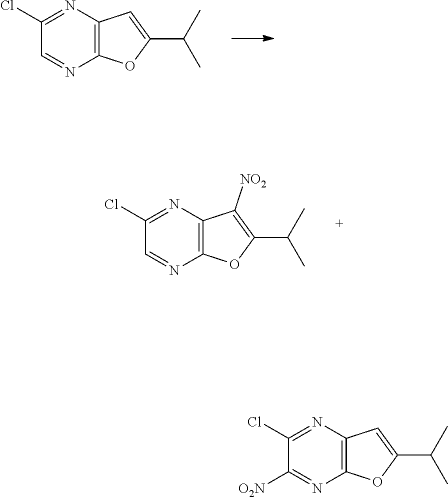 [SMILES: CC(C)c1cc2nc(Cl)cnc2o1 ; CC(C)c1oc2ncc(Cl)nc2c1[N+](=O)[O-] ; CC(C)c1cc2nc(Cl)c([N+](=O)[O-])nc2o1]

To stirred acetic anhydride (10.820 g, 10 mL, 105.99 mmol) in an ice bath was added nitric acid (2.9187 g, 2.3 mL of 90% w/w, 41.687 mmol) dropwise and then the mixture was stirred at room temperature for 10 minutes. The pre-prepared nitrate was added dropwise to a stirred mixture of acetic anhydride (25 mL) and 2-chloro-6-isopropyl-furo[2,3-b]pyrazine (2.1 g, 10.680 mmol) at 0° C. The reaction was slowly warmed to room temperature. The mixture was stirred at rt for 0.5 h. After 2 hours, 2 mL of 90% HNO₃ was added dropwise at room temperature to the reaction mixture. The reaction was stirred overnight at room temperature and then was diluted with ice cold water (200 mL). The aqueous phase was extracted with DCM (2×50 mL), dried over sodium sulfate, and evaporated to dryness. The residue was purified by silica gel chromatography (0-30% of ethyl acetate in hexane) to give two fractions after evaporation: fraction 1:2-chloro-6-isopropyl-3-nitro-furo[2,3-b]pyrazine (0.9 g, 17%), ESI-MS m/z calc. 241.0254, found 242.3 (M+1)⁺; Retention time: 5.35 minutes; and fraction 2:1/1 mixture of the previous compound and 2-chloro-6-isopropyl-7-nitro-furo[2,3-b]pyrazine (0.45 g, 16%). ESI-MS m/z calc. 241.0254, found 242.3 (M+1)⁺; Retention time: 4.72 minutes. LC method D.

### Step 2: 2-Chloro-6-isopropyl-furo[2,3-b]pyrazin-3-amine and tert-butyl N-(2-chloro-6-isopropyl-furo[2,3-b]pyrazin-7-yl)carbamate

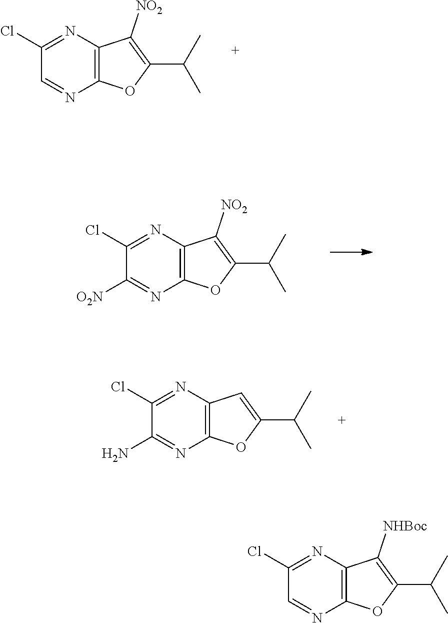 [SMILES: CC(C)c1oc2ncc(Cl)nc2c1[N+](=O)[O-] ; CC(C)c1oc2nc([N+](=O)[O-])c(Cl)nc2c1[N+](=O)[O-] ; CC(C)c1cc2nc(Cl)c(N)nc2o1 ; CC(C)c1oc2ncc(Cl)nc2c1NC(=O)OC(C)(C)C]

To a solution of a 1:1 mixture of 2-chloro-6-isopropyl-3-nitro-furo[2,3-b]pyrazine (225 mg, 0.7449 mmol) and 2-chloro-6-isopropyl-7-nitro-furo[2,3-b]pyrazine (225 mg, 0.7449 mmol) (total 0.45 g) in THE (8 mL) and t-BuOH (1 mL) were added activated Ni (90 mg, 1.5334 mmol) and di-tert-butyl dicarbonate (1.3 g, 1.3684 mL, 5.9566 mmol), and the mixture was stirred 4 hour under hydrogen atmosphere (1 atm) at room temperature. The suspension was filtered through zeolite and evaporated to dryness. The residue was purified by silica gel chromatography (0-30% Ethyl Acetate in Hexane) to give 2-chloro-6-isopropyl-furo[2,3-b]pyrazin-3-amine (96 mg, 58%) ESI-MS m/z calc. 211.0512, found 212.4 (M+1)⁺; Retention time: 4.33 minutes, ¹H NMR (500 MHz, CDCl₃) δ 6.38 (s, 1H), 4.91 (s, 2H), 3.05 (td, J=13.7, 6.8 Hz, 1H), 1.33 (d, J=6.9 Hz, 6H) as colorless solid; and tert-butyl N-(2-chloro-6-isopropyl-furo[2,3-b]pyrazin-7-yl)carbamate (203 mg, 70%), ESI-MS m/z calc. 311.1037, found 312.4 (M+1)⁺; Retention time: 5.48 minutes, ¹H NMR (500 MHz, CDCl₃) δ 8.17 (s, 1H), 6.04 (s, 1H), 3.42-3.32 (m, 1H), 1.54-1.48 (m, 9H), 1.40 (d, J=6.9 Hz, 6H) as light yellow solid. LC method D.

### Step 3: Methyl 7-(tert-butoxycarbonylamino)-6-isopropyl-furo[2,3-b]pyrazine-2-carboxylate

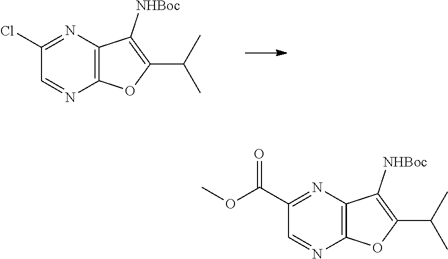 [SMILES: CC(C)c1oc2ncc(Cl)nc2c1NC(=O)OC(C)(C)C ; COC(=O)c1cnc2oc(C(C)C)c(NC(=O)OC(C)(C)C)c2n1]

A mixture of tert-butyl N-(2-chloro-6-isopropyl-furo[2,3-b]pyrazin-7-yl)carbamate (0.2 g, 0.5132 mmol), Pd(dppf)₂·DCM (55 mg, 0.0673 mmol) and TEA (290.40 mg, 0.4 mL, 2.8698 mmol) in MeOH (15 mL) was charged in a steel bomb equipped with mechanical stirrer, and the reaction was purged with carbon monoxide three times. The reaction mixture was heated to 70° C. under 120 psi of carbon monoxide and stirred for 6 h. The reaction mixture was allowed to cool to rt. The reaction mixture was filtered through a pad of celite, washed with ethyl acetate, filtrate was concentrated in vacuo. The residue was purified by silica gel chromatography in 30% ethyl acetate in hexanes to give methyl 7-(tert-butoxycarbonylamino)-6-isopropyl-furo[2,3-b]pyrazine-2-carboxylate (130 mg, 68%) as a yellow-green solid. ¹H NMR (500 MHz, CDCl₃) δ 8.99 (s, 1H), 6.42 (s, 1H), 4.03 (s, 3H), 3.40 (hept, J=6.9 Hz, 1H), 1.48 (s, 9H), 1.40 (d, J=6.9 Hz, 6H). ESI-MS m/z calc. 335.14813, found 336.4 (M+1)⁺; Retention time: 4.95 minutes; LC method D.

### Step 4: tert-Butyl N-(2-formyl-6-isopropyl-furo[2,3-b]pyrazin-7-yl)carbamate

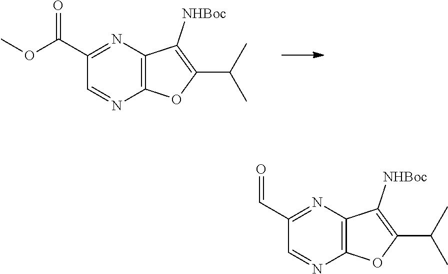 [SMILES: COC(=O)c1cnc2oc(C(C)C)c(NC(=O)OC(C)(C)C)c2n1 ; CC(C)c1oc2ncc(C=O)nc2c1NC(=O)OC(C)(C)C]

To a stirred solution of methyl 7-(tert-butoxycarbonylamino)-6-isopropyl-furo[2,3-b]pyrazine-2-carboxylate (140 mg, 0.3757 mmol) in anhydrous DCM (4 mL) was added DIBAL in DCM (0.8 mL of 1 M, 0.8000 mmol) at −78° C. The reaction mixture was stirred for 2 h at −78° C. Another 0.7 mL of DIBAL solution was added and the reaction was slowly allowed to warm up to −40° C. over 2 hours, then the reaction was slowly allowed to warm up to −20° C. over 2 hours. The reaction was cooled to −70° C. and quenched with 0.2 mL of MeOH and 0.2 mL of water. The reaction was warmed to 0° C. and filtered through zeolite. The filtrate was evaporated and combined with another crude reaction for purification by silica gel chromatography (0-15% ethyl acetate in hexane) to give tert-butyl N-(2-formyl-6-isopropyl-furo[2,3-b]pyrazin-7-yl)carbamate (130 mg) as a light yellow solid. ¹H NMR (500 MHz, CDCl₃) δ 10.21 (s, 1H), 8.88 (s, 1H), 6.16 (s, 1H), 3.55-3.35 (m, 1H), 1.81-1.31 (m, 15H). ESI-MS m/z calc. 305.13754, found 306.4 (M+1)⁺; Retention time: 2.41 minutes; LC method H.

### Step 5: 3-[[4-[(2R)-2-[[7-(tert-Butoxycarbonylamino)-6-isopropyl-furo[2,3-b]pyrazin-2-yl]methylamino]-3-(1-methylcyclopropyl)propoxy]-6-(2,6-dimethylphenyl)pyrimidin-2-yl]sulfamoyl]benzoic acid

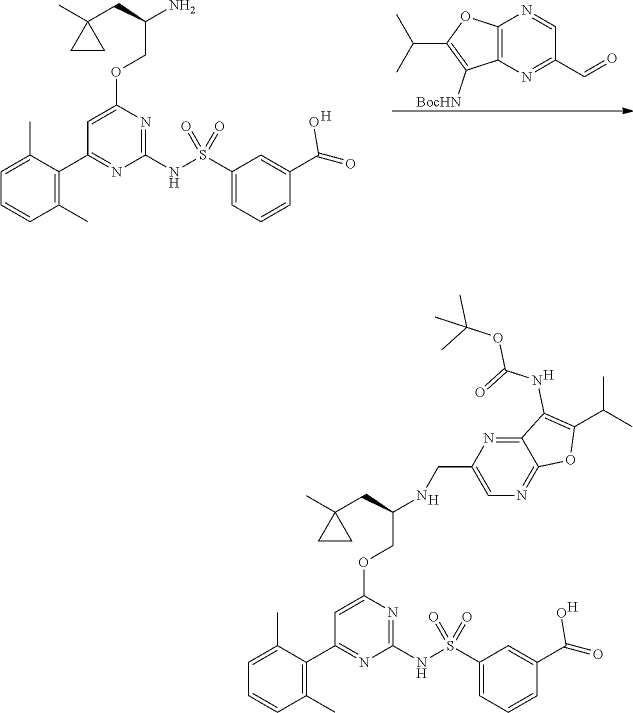

A 4 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (106 mg, 0.1938 mmol), tert-butyl N-(2-formyl-6-isopropyl-furo[2,3-b]pyrazin-7-yl)carbamate (59.2 mg, 0.1939 mmol), anhydrous DCM (0.89 mL), and acetic acid (22.1 μL, 0.3886 mmol). The mixture was cooled down in an ice bath. DIEA (101.4 μL, 0.5821 mmol) was added, followed by sodium triacetoxyborohydride (207.3 mg, 0.9781 mmol), and the reaction was vigorously stirred at 0° C. for 4 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) followed by extraction with ethyl acetate, drying over MgSO₄, filtration, and concentration under reduced pressure provided 3-[[4-[(2R)-2-[[7-(tert-butoxycarbonylamino)-6-isopropyl-furo[2,3-b]pyrazin-2-yl]methylamino]-3-(1-methylcyclopropyl)propoxy]-6-(2,6-dimethylphenyl)pyrimidin-2-yl]sulfamoyl]benzoic acid (hydrochloride salt) (104.7 mg, 59%) as a white solid. ESI-MS m/z calc. 799.3363, found 800.1 (M+1)⁺; Retention time: 1.48 minutes. LC method A.

### Step 6: tert-Butyl N-[2-[[(11R)-6-(2,6-dimethylphenyl)-11-[(1-methylcyclopropyl)methyl]-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-6-isopropyl-furo[2,3-b]pyrazin-7-yl]carbamate (Compound I-210

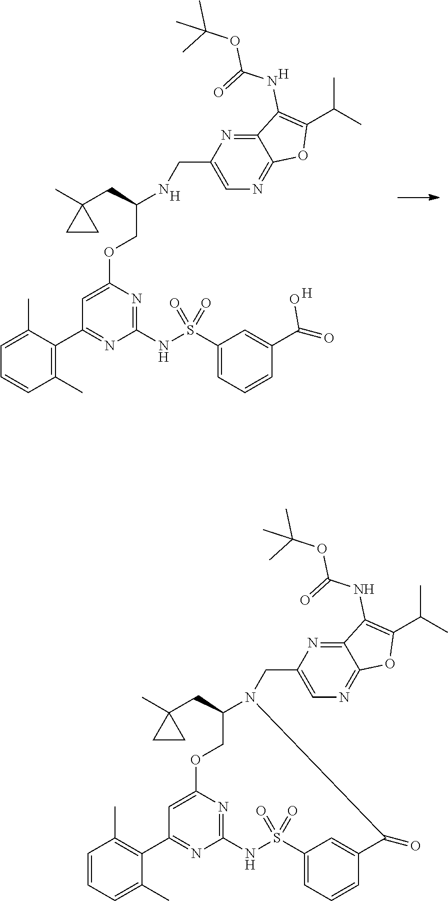

3-[[4-[(2R)-2-[[7-(tert-Butoxycarbonylamino)-6-isopropyl-furo[2,3-b]pyrazin-2-yl]methylamino]-3-(1-methylcyclopropyl)propoxy]-6-(2,6-dimethylphenyl)pyrimidin-2-yl]sulfamoyl]benzoic acid (hydrochloride salt) (104.7 mg, 0.1152 mmol) was combined with CDMT (31.8 mg, 0.1811 mmol) in DMF (8.6 mL) and cooled to 0° C. N-methylmorpholine (38.0 μL, 0.3456 mmol) was added by syringe and the reaction was stirred at 0° C. for 30 minutes. The ice bath was then removed and stirring was continued for an additional 5 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min). The desired fractions were collected, extracted with ethyl acetate, dried over sodium sulfate, filtered, and concentrated to give tert-butyl N-[2-[[(11R)-6-(2,6-dimethylphenyl)-11-[(1-methylcyclopropyl)methyl]-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-6-isopropyl-furo[2,3-b]pyrazin-7-yl]carbamate (53.1 mg, 55%) as a white solid. ¹H NMR (400 MHz, MeOD) δ 8.69 (s, 1H), 8.38 (s, 1H), 8.02 (dt, J=7.8, 1.5 Hz, 1H), 7.74 (dt, J=7.6, 1.5 Hz, 1H), 7.67 (t, J=7.7 Hz, 1H), 7.27 (t, J=7.6 Hz, 1H), 7.14 (d, J=7.7 Hz, 2H), 6.29 (s, 1H), 5.61-5.52 (m, 1H), 5.12 (d, J=15.3 Hz, 1H), 4.53-4.36 (m, 3H), 3.40-3.33 (m, 1H), 2.10 (s, 6H), 1.82 (d, J=15.2 Hz, 1H), 1.68-1.52 (m, 2H), 1.45 (s, 9H), 1.39 (d, J=4.8 Hz, 3H), 1.38 (d, J=4.8 Hz, 3H), 0.50 (s, 3H), 0.45-0.35 (m, 1H), 0.29-0.20 (m, 1H), 0.20-0.11 (m, 1H), 0.11-0.03 (m, 1H). ESI-MS m/z calc. 781.32574, found 782.1 (M+1)⁺; Retention time: 2.0 minutes. LC method A.

### Example 95: Preparation of Compound I-220

### Step 1: (2R)-2-Amino-3-[(2R)-tetrahydropyran-2-yl]propan-1-ol

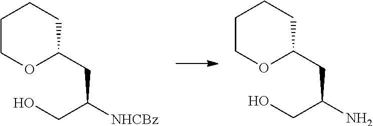

Nitrogen was bubbled to a stirred solution of benzyl N-[(1R)-1-(hydroxymethyl)-2-[(2R)-tetrahydropyran-2-yl]ethyl]carbamate (2.99 g, 10.182 mmol) in methanol (60 mL). Then palladium hydroxide on carbon (1.1 g, 20% w/w, 1.5666 mmol) was added and nitrogen bubbling was kept on going for an additional 10 minutes. Then hydrogen was bubbled to the mixture for 10 minutes and the reaction was stirred overnight at room temperature under hydrogen atmosphere. The reaction was then filtered on a pad of celite, washed with ethyl acetate (3×15 mL), then concentrated under reduced pressure to afford crude (2R)-2-amino-3-[(2R)-tetrahydropyran-2-yl]propan-1-ol (1.61 g, 94%) as a colorless oil that was directly used in the next step without further purification. ¹H NMR (400 MHz, DMSO-d6) δ 4.58 (br. s, 1H), 3.89-3.79 (m, 1H), 3.53-2.98 (m, 4H, overlapped with water), 2.89-2.71 (m, 1H), 1.82-1.67 (m, 1H), 1.62-1.52 (m, 1H), 1.48-1.25 (m, 5H), 1.17-1.03 (m, 1H). (2H missing, labile H). ESI-MS m/z calc. 159.12593, found 160.2 (M+1)⁺; Retention time: 0.42 minutes; LC method I.

### Step 2: (2R)-2-Amino-3-[(2R)-tetrahydropyran-2-yl]propan-1-ol

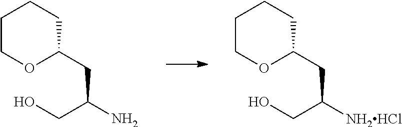

A stirred solution of (2R)-2-amino-3-[(2R)-tetrahydropyran-2-yl]propan-1-ol (1.66 g, 9.9042 mmol) in 1,4-dioxane (20 mL) was cooled down to 0° C. then a solution of hydrogen chloride (5 mL of 4 M, 20.000 mmol) was added dropwise. The mixture was then allowed to reach room temperature and stirred overnight at room temperature. The reaction was concentrated under reduced pressure then co-evaporated with methyl-tertbutylether (3×15 mL). The residue was then partitioned between water (50 mL) and methyl-tertbutylether (30 mL) and the layers were separated. The aqueous layer was then evaporated under reduced pressure and co-evaporated with acetonitrile (4×10 mL) then freeze-dried to afford (2R)-2-amino-3-[(2R)-tetrahydropyran-2-yl]propan-1-ol (hydrochloride salt) (2.01 g, 98%) as a yellow oil. ¹H NMR (400 MHz, DMSO-d6) δ 7.86 (br. s, 3H), 5.28 (br. s, 1H), 3.89-3.79 (m, 1H), 3.59 (dd, J=11.5, 3.2 Hz, 1H), 3.46 (dd, J=11.2, 6.1 Hz, 1H), 3.41-3.26 (m, 2H, overlapped with water), 3.23-3.12 (m, 1H), 1.82-1.69 (m, 1H), 1.68-1.51 (m, 3H), 1.50-1.35 (m, 3H), 1.21-1.07 (m, 1H). ESI-MS m/z calc. 159.12593, found 160.2 (M+1)⁺; Retention time: 0.71 minutes; LC method J.

### Step 3: 3-[[4-[(2R)-2-Amino-3-[(2R)-tetrahydropyran-2-yl]propoxy]-6-(2,6-dimethylphenyl)pyrimidin-2-yl]sulfamoyl]benzoic acid

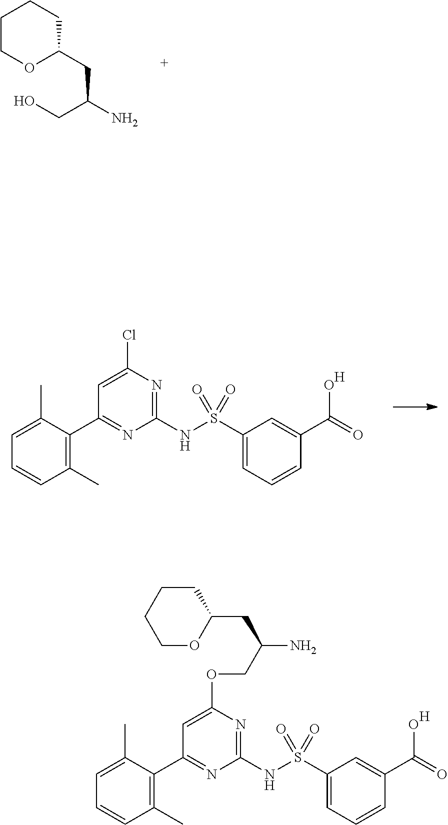

A solution of (2R)-2-amino-3-[(2R)-tetrahydropyran-2-yl]propan-1-ol (hydrochloride salt) (256 mg, 1.2428 mmol) in anhydrous DMF (1.5 mL) was added to a solution of 3-[[4-chloro-6-(2,6-dimethylphenyl)pyrimidin-2-yl]sulfamoyl]benzoic acid (460 mg, 1.1008 mmol) in 2-methyltetrahydrofuran (13 mL). The resulting solution was stirred at room temperature for 15 minutes then cooled down to 10-15° C. and sodium tert-butoxide (265 mg, 2.7574 mmol) was added. The reaction was stirred at 10-15° C. for 30 minutes after which time supplementary sodium tert-butoxide (265 mg, 2.7574 mmol) was added. The reaction was stirred at 10-15° C. for 45 minutes. Supplementary sodium tert-butoxide (265 mg, 2.7574 mmol) was added and the reaction was stirred at 10-15° C. for 45 minutes. Supplementary sodium tert-butoxide (265 mg, 2.7574 mmol) was added and the reaction was stirred at 10-15° C. for 1 hour. The reaction was then cooled down to 0° C. and quenched by the addition of an aqueous solution of 1 N hydrochloric acid (50 mL). The mixture was vigorously stirred for 30 minutes, then the layers were separated, and the aqueous layer was extracted with 2-methyltetrahydrofuran (5×15 mL) and the combined organic layers were washed with brine (100 mL), dried over magnesium sulfate, filtered, and concentrated under reduced pressure to afford a beige foam which was stirred overnight with ethyl acetate (100 mL). The solvent was removed under reduced pressure and the white foam was purified by reverse-phase chromatography on C₁₈ (column: 50 g HP Gold C₁₈; gradient: 5 to 100% methanol in water containing 0.1% v/v of hydrochloric acid; 15 CV). The desired fractions were concentrated under reduced pressure and the remaining water was co-evaporated with methanol (5×20 mL), then with acetonitrile (4×10 mL) and freeze-dried to afford 3-[[4-[(2R)-2-amino-3-[(2R)-tetrahydropyran-2-yl]propoxy]-6-(2,6-dimethylphenyl)pyrimidin-2-yl]sulfamoyl]benzoic acid (hydrochloride salt) (367 mg, 57%) as a white fluffy powder. ¹H NMR (400 MHz, DMSO-d6) δ 13.29 (br. s, 1H), 8.45 (s, 1H), 8.22-7.95 (m, 5H), 7.70 (t, J=7.8 Hz, 1H), 7.31-7.21 (m, 1H), 7.17-7.07 (m, 2H), 6.30 (br. s, 1H), 4.33 (dd, J=12.0, 2.9 Hz, 1H), 4.21 (dd, J=11.7, 6.8 Hz, 1H), 3.93-3.84 (m, 1H), 3.72-3.63 (m, 1H), 3.59-3.30 (m, 2H, overlapped with water), 2.01 (br. s, 6H), 1.82-1.69 (m, 3H), 1.60 (d, J=12.7 Hz, 1H), 1.53-1.39 (m, 3H), 1.28-1.13 (m, 1H). (1H missing, labile H). ESI-MS m/z calc. 540.2043, found 541.1 (M+1)⁺; Retention time: 2.41 minutes; LC method J.

### Step 4: 3-[[4-[(2R)-2-[(6-tert-Butylfuro[2,3-b]pyrazin-2-yl)methylamino]-3-[(2R)-tetrahydropyran-2-yl]propoxy]-6-(2,6-dimethylphenyl)pyrimidin-2-yl]sulfamoyl]benzoic acid

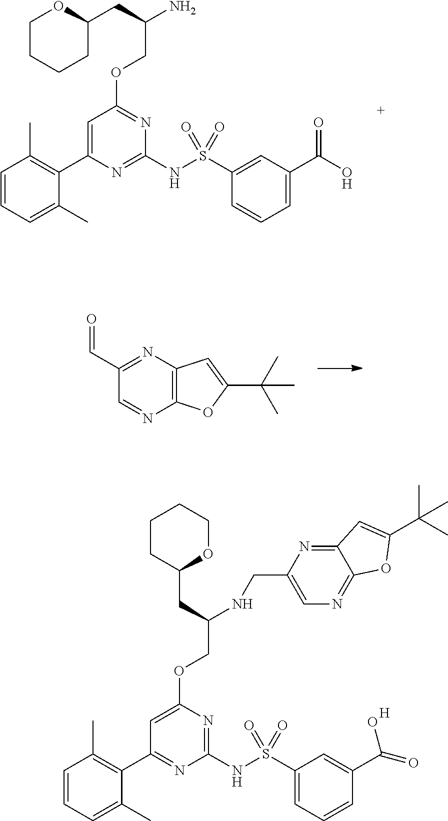

3-[[4-[(2R)-2-Amino-3-[(2R)-tetrahydropyran-2-yl]propoxy]-6-(2,6-dimethylphenyl)pyrimidin-2-yl]sulfamoyl]benzoic acid (hydrochloride salt) (42 mg, 0.07278 mmol) was combined with 6-tert-butylfuro[2,3-b]pyrazine-2-carbaldehyde (16.5 mg, 0.08079 mmol) in DCM (0.4 mL). acetic acid (7 μL, 0.1231 mmol) was added and the reaction mixture was cooled to 0° C. in an ice water bath. DIPEA (40 μL, 0.2296 mmol) was added and after stirring at this temperature for 20 minutes sodium triacetoxyborohydride (80 mg, 0.3775 mmol) was added. Stirring at 0° C. was continued for 3 hours. The reaction mixture was then quenched with 0.2 mL 3M HCl, diluted with methanol and DMSO, filtered, and purified by preparative HPLC (1-99% ACN in water, HCl modifier, 15 minute run) to give 3-[[4-[(2R)-2-[(6-tert-butylfuro[2,3-b]pyrazin-2-yl)methylamino]-3-[(2R)-tetrahydropyran-2-yl]propoxy]-6-(2,6-dimethylphenyl)pyrimidin-2-yl]sulfamoyl]benzoic acid (hydrochloride salt) (31 mg, 56%). ESI-MS m/z calc. 728.2992, found 729.7 (M+1)⁺; Retention time: 0.63 minutes; LC method B.

### Step 5: (11R)-12-[(6-tert-Butylfuro[2,3-b]pyrazin-2-yl)methyl]-6-(2,6-dimethylphenyl)-2,2-dioxo-11-[[(2R)-tetrahydropyran-2-yl]methyl]-9-oxa-2λ⁶-thia-3,5,12,19-tetrazatricyclo[12.3.1.14,8]nonadeca-1(18),4(19),5,7,14,16-hexaen-13-one (Compound I-220

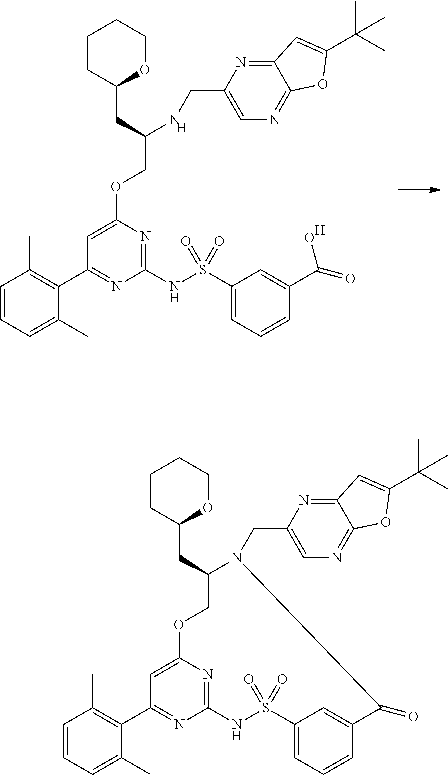

3-[[4-[(2R)-2-[(6-tert-Butylfuro[2,3-b]pyrazin-2-yl)methylamino]-3-[(2R)-tetrahydropyran-2-yl]propoxy]-6-(2,6-dimethylphenyl)pyrimidin-2-yl]sulfamoyl]benzoic acid (hydrochloride salt) (31 mg, 0.04051 mmol) was combined with CDMT (9 mg, 0.05126 mmol) and dissolved in DMF (3.5 mL). DIPEA (50 μL, 0.2871 mmol) was added and the reaction mixture was stirred for 48 hours at room temperature. The reaction mixture was then concentrated by rotary evaporation under reduced pressure and the resulting residue was dissolved in 1:1 DMSO/methanol, filtered and purified by preparative HPLC (1-99% ACN in water, HCl modifier, 15 minute run) to give (11R)-12-[(6-tert-butylfuro[2,3-b]pyrazin-2-yl)methyl]-6-(2,6-dimethylphenyl)-2,2-dioxo-11-[[(2R)-tetrahydropyran-2-yl]methyl]-9-oxa-2λ⁶-thia-3,5,12,19-tetrazatricyclo[12.3.1.14,8]nonadeca-1(18),4(19),5,7,14,16-hexaen-13-one (20.5 mg, 70%) ESI-MS m/z calc. 710.28864, found 711.8 (M+1)⁺; Retention time: 2.01 minutes; LC method A. ¹H NMR (400 MHz, Chloroform-d) δ 8.66 (t, J=2.1 Hz, 1H), 8.42 (s, 1H), 8.13 (d, J=7.9 Hz, 1H), 7.83 (dt, J=7.8, 1.3 Hz, 1H), 7.66 (t, J=7.8 Hz, 1H), 7.21 (t, J=7.6 Hz, 1H), 7.05 (d, J=7.5 Hz, 2H), 6.61 (s, 1H), 6.23 (d, J=3.1 Hz, 1H), 5.37 (dd, J=10.8, 3.6 Hz, 1H), 5.32 (d, J=15.0 Hz, 1H), 4.45-4.19 (m, 3H), 3.88 (d, J=10.5 Hz, 1H), 3.19 (td, J=11.1, 3.7 Hz, 1H), 3.04-2.94 (m, 1H), 2.02 (d, J=2.5 Hz, 6H), 1.96 (dd, J=15.5, 7.8 Hz, 1H), 1.77-1.68 (m, 2H), 1.51-1.44 (m, 2H), 1.42 (s, 9H), 1.33 (dt, J=12.4, 4.2 Hz, 1H), 1.27-1.20 (m, 1H), 1.08 (qd, J=12.9, 3.9 Hz, 1H).

### Example 96: Preparation of Compound I-225

### Step 1: Methyl 5-hydroxy-6-iodo-pyridine-2-carboxylate

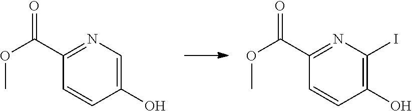

To a flask (500 mL) were added methyl 5-hydroxypyridine-2-carboxylate (5 g, 32.651 mmol), sodium carbonate (7.1 g, 66.989 mmol), and water (180 mL). To the stirring mixture was added iodine (8.6 g, 33.884 mmol). The mixture was stirred at room temperature for 2 hours. Then, the pH of the solution was adjusted to ˜4 by HCl aqueous solution (1 N) and diluted with EtOAc (500 mL). The resulted solution was stirred for 5 min. The layers were separated, and the organic layer was washed with saturated Na₂S₂O₃ aqueous solution (300 mL) and brine (200 mL), respectively. The organic layer was dried over sodium sulfate, filtered, and concentrated under reduced pressure. The residue was purified by silica gel chromatography (0-20% ethyl acetate in DCM) to afford methyl 5-hydroxy-6-iodo-pyridine-2-carboxylate (3.51 g, 35%) as a white solid after evaporation. ¹H NMR (500 MHz, CDCl₃) δ 8.01 (d, J=8.3 Hz, 1H), 7.29 (d, J=8.3 Hz, 1H), 6.14 (s, 1H), 3.97 (s, 3H). ESI-MS m/z calc. 278.93924, found 280.2 (M+1)⁺; Retention time: 2.45 minutes; LC method D.

### Step 2: Methyl 2-(1-methylcyclopropyl)furo[3,2-b]pyridine-5-carboxylate

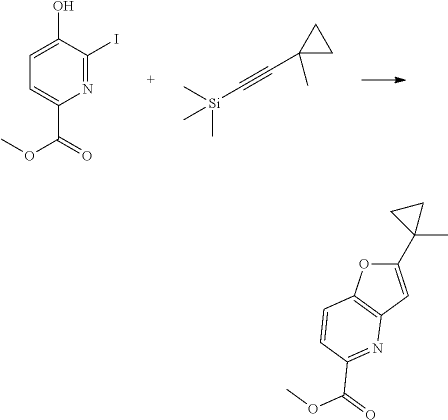

To a pressure vial were added methyl 5-hydroxy-6-iodo-pyridine-2-carboxylate (3.5 g, 11.289 mmol), bis(triphenylphosphine)palladium(II) dichloride (620 mg, 0.8833 mmol), CuI (340 mg, 1.7852 mmol), KF (2.1 g, 36.147 mmol) in DMF (35 mL), TEA (10 mL) and trimethyl-[2-(1-methylcyclopropyl)ethynyl]silane (4.8 g, 26.788 mmol). The reaction was stirred at 80° C. for 12 hours. The reaction mixture was allowed to cool down, diluted with ethyl acetate (300 mL) and washed with water (2×300 mL). The organic layer was washed with brine (100 mL), dried over sodium sulfate, filtered, and concentrated in vacuo. The orange solid residue was purified by silica-gel chromatography to give (methyl 2-(1-methylcyclopropyl)furo[3,2-b]pyridine-5-carboxylate (1.8 g, 62%) as a yellow solid. ¹H NMR (500 MHz, CDCl₃) δ 8.05 (d, J=8.4 Hz, 1H), 7.66 (d, J=8.3 Hz, 1H), 6.70 (s, 1H), 4.03 (s, 3H), 1.53 (s, 3H), 1.35-1.29 (m, 2H), 0.95-0.89 (m, 2H). ¹³C NMR (126 MHz, Chloroform-d) δ 169.87, 166.09, 149.85, 148.85, 143.47, 120.19, 117.00, 101.93, 52.84, 21.19, 16.25, 15.39. ESI-MS m/z calc. 231.08954, found 232.2 (M+1)⁺; Retention time: 4.05 minutes; LC method D.

### Step 3: 2-(1-Methylcyclopropyl)furo[3,2-b]pyridine-5-carbaldehyde

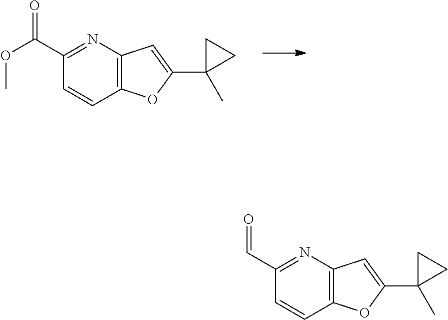 [SMILES: COC(=O)c1ccc2oc(C3(C)CC3)cc2n1 ; CC1(c2cc3nc(C=O)ccc3o2)CC1]

To a stirred solution of methyl 2-(1-methylcyclopropyl)furo[3,2-b]pyridine-5-carboxylate (1.8 g, 7.0055 mmol) in DCM (60 mL) was added DIBAL (12 mL of 1 M, 12.0 mmol) in DCM at −78° C. The reaction mixture was stirred for 2 h, quenched with a mixture of MeOH (3 mL) and water (3 mL) at −78° C. and the reaction was allowed to warm to rt. DCM (100 mL) was added and the solid was filtered. The cake was washed with DCM (3×50 mL). The organic layer from the filtrate was separated, washed with brine (2×50 mL), dried over sodium sulfate, filtered, and concentrated in vacuo. The residue was purified by silica gel chromatography, loaded with DCM, eluted with 20% ethyl acetate in hexanes. The desired product fractions were combined, concentrated in vacuo and dried overnight to give 2-(1-methylcyclopropyl)furo[3,2-b]pyridine-5-carbaldehyde (1.05 g, 73%) as an off-white solid. ¹H NMR (500 MHz, CDCl₃) δ 10.11 (s, 1H), 7.87 (d, J=8.4 Hz, 1H), 7.69 (d, J=8.4 Hz, 1H), 6.69 (s, 1H), 1.55 (s, 3H), 1.36-1.29 (m, 2H), 0.97-0.90 (m, 2H). ESI-MS m/z calc. 201.07898, found 202.0 (M+1)⁺; Retention time: 2.04 minutes; LC method H.

### Step 4: 3-[[4-[(2R)-3-(1-Bicyclo[1.1.1]]pentanyl)-2-[[2-(1-methylcyclopropyl)furo[3,2-b]pyridin-5-yl]methylamino]propoxy]-6-(2,6-dimethylphenyl)pyrimidin-2-yl]sulfamoyl]benzoic acid

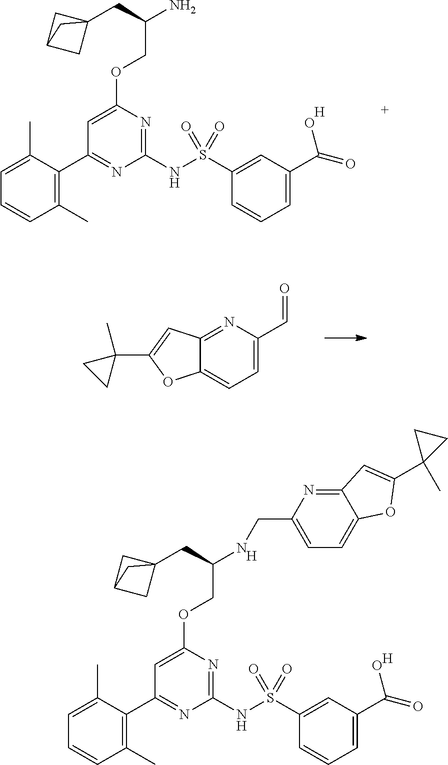

3-[[4-[(2R)-2-Amino-3-(1-bicyclo[1.1.1]pentanyl)propoxy]-6-(2,6-dimethylphenyl)pyrimidin-2-yl]sulfamoyl]benzoic acid (hydrochloride salt) (65 mg, 0.1046 mmol) was combined with 2-(1-methylcyclopropyl)furo[3,2-b]pyridine-5-carbaldehyde (23 mg, 0.1143 mmol) in DCM. Acetic acid (10 μL, 0.1758 mmol) was added and the reaction mixture was cooled to 0° C. in an ice water bath. DIPEA (50 μL, 0.2871 mmol) was added and after stirring at this temperature for 20 minutes, sodium triacetoxyborohydride (120 mg, 0.5662 mmol) was added. Stirring at 0° C. was continued for 2 hours. The reaction mixture was then quenched with 0.2 mL 3M HCl, diluted with methanol and DMSO until the reaction mixture became homogeneous, filtered, and purified by preparative HPLC (1-99% ACN in water, HCl modifier, 15 minute run) to give 3-[[4-[(2R)-3-(1-bicyclo[1.1.1]pentanyl)-2-[[2-(1-methylcyclopropyl)furo[3,2-b]pyridin-5-yl]methylamino]propoxy]-6-(2,6-dimethylphenyl)pyrimidin-2-yl]sulfamoyl]benzoic acid (hydrochloride salt) (49.3 mg, 63%). ESI-MS m/z calc. 707.2778, found 708.8 (M+1)⁺; Retention time: 0.62 minutes; LC method B.

### Step 5: (11R)-11-(1-Bicyclo[1.1.1]pentanylmethyl)-6-(2,6-dimethylphenyl)-12-[[2-(1-methylcyclopropyl)furo[3,2-b]pyridin-5-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-225

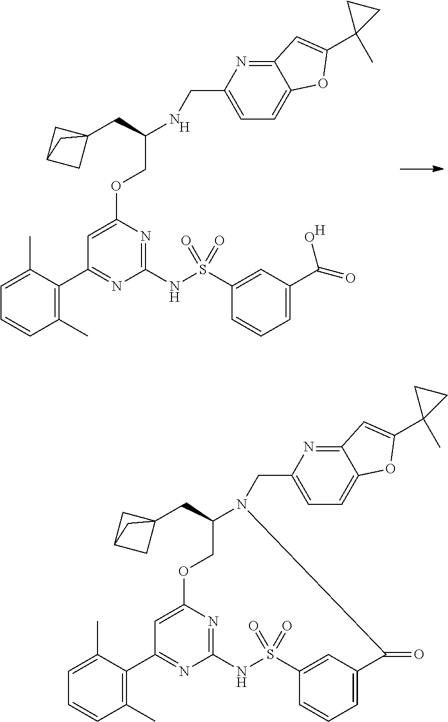

3-[[4-[(2R)-3-(1-Bicyclo[1.1.1]pentanyl)-2-[[2-(1-methylcyclopropyl)furo[3,2-b]pyridin-5-yl]methylamino]propoxy]-6-(2,6-dimethylphenyl)pyrimidin-2-yl]sulfamoyl]benzoic acid (hydrochloride salt) (49.3 mg, 0.06624 mmol) was combined with CDMT (14 mg, 0.07974 mmol) in DMF. N-methylmorpholine (50 μL, 0.4548 mmol) was added by syringe and the reaction was stirred at room temperature for the indicated time. Volatiles were then removed by rotary evaporation under reduced pressure, and the resulting residue was dissolved in DMSO/methanol, filtered, and purified by preparative HPLC (1-99% ACN in water, HCl modifier) to give (11R)-11-(1-bicyclo[1.1.1]pentanylmethyl)-6-(2,6-dimethylphenyl)-12-[[2-(1-methylcyclopropyl)furo[3,2-b]pyridin-5-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (28.6 mg, 61%). ESI-MS m/z calc. 689.2672, found 690.7 (M+1)⁺; Retention time: 1.9 minutes; LC method A. ¹H NMR (400 MHz, Chloroform-d) δ 8.72 (s, 1H), 8.64 (s, 1H), 8.10 (dt, J=8.0, 1.5 Hz, 1H), 7.89 (dt, J=7.7, 1.4 Hz, 1H), 7.65 (t, J=7.8 Hz, 1H), 7.40 (s, 1H), 7.20 (t, J=7.6 Hz, 1H), 7.05 (d, J=7.6 Hz, 2H), 6.66 (s, 1H), 6.20 (s, 1H), 5.48 (d, J=15.0 Hz, 1H), 5.13 (s, 1H), 4.38 (t, J=11.2 Hz, 1H), 4.21 (s, 2H), 1.99 (s, 6H), 1.78 (d, J=15.0 Hz, 1H), 1.55-1.50 (m, 4H), 1.31 (s, 2H), 0.91 (s, 2H), 0.47 (s, 3H), 0.35 (dt, J=9.7, 5.0 Hz, 1H), 0.22 (dt, J=9.8, 5.0 Hz, 1H), 0.11 (dt, J=9.4, 4.9 Hz, 1H), 0.06-0.00 (m, 1H).

### Example 97: Preparation of Compound I-227

### Step 1: Trimethyl-[2-(1-methylcyclopropyl)ethynyl]silane

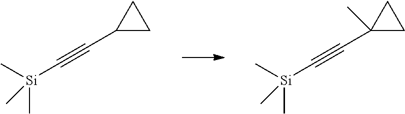

To a solution of 2-cyclopropylethynyl(trimethyl)silane (8.54 g, 10.047 mL, 61.758 mmol) in Et₂O (70 mL) at room temperature was slowly added n-BuLi in hexanes (30 mL of 2.5 M, 75.000 mmol) over 20 minutes. The reaction mixture was stirred at room temperature for 18 hours, then the reaction was cooled down to −15° C. in a dry-ice/acetone bath. To the reaction mixture was slowly added dropwise Mel (9.5760 g, 4.2 mL, 67.466 mmol) over 10 minutes. The resulting solution was stirred at −10° C. for 10 minutes, and then at 0° C. for 1 hour. The reaction mixture was allowed to warm to room temperature for 1 hour. The reaction mixture was diluted with diethyl ether (50 mL) and water (50 mL) and brine (50 mL) were added. The phases were separated, and the aqueous phase was extracted with diethyl ether (3×20 mL). The combined organic layers were washed with brine (50 mL), dried over anhydrous sodium sulfate, filtered, and concentrated under reduced pressure. Purification by silica gel chromatography (Eluent: 100% Heptane) afforded trimethyl-[2-(1-methylcyclopropyl)ethynyl]silane (2.63 g, 19%) as a light yellow liquid. GC-MS m/z calc. 152.1021, found 152.00 (M), retention time 5.041 minutes. GC-MS method: Column HP 5 ms Ultra Inert 30 m×250 m×0.25 m, control mode: flow: 3.3 mL/min, split ratio mode: 33:1, split flow: 165 mL/min., carrier gas: He. Injector temperature 250° C., FID temperature 300° C., MSD transfer line 250° C., solvent delay: 3 min. Oven temperature: isotherm at 40° C. for 1 min, then linear heating at 10° C./min until 100° C., then 20° C./min until 220° C. then isotherm 220° C. for 4 min. Run time 17 min.

### Step 2: 6-Chloro-3-[2-(1-methylcyclopropyl)ethynyl]pyrazin-2-amine

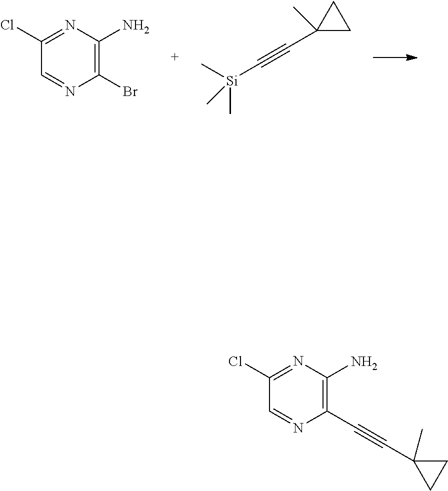

To a sealed tube equipped with a stir bar were added copper(I) iodide (221 mg, 1.1604 mmol), bis(triphenylphosphine)palladium(II) chloride (330 mg, 0.4702 mmol), triphenylphosphine (234 mg, 0.8922 mmol), cesium fluoride (4.6 g, 30.282 mmol), 3-bromo-6-chloro-pyrazin-2-amine (3.15 g, 15.112 mmol), poly(ethylene glycol) 200 MW (3.0 mL), triethylamine (25 mL) and water (1.5 mL). The vial was capped and the mixture sparged with nitrogen for 15 minutes while being stirred at room temperature. Trimethyl-[2-(1-methylcyclopropyl)ethynyl]silane (2.63 g, 11.621 mmol) was then added and the tube was sealed. The reaction mixture was allowed to stir at 80° C. for 17 hours. The mixture was allowed to cool to room temperature then was diluted with ethyl acetate (50 mL), water (50 mL) and brine (50 mL). The phases were separated, and the organic layer was washed with a 50% aqueous saturated sodium chloride solution (2×50 mL). The combined aqueous layers were extracted with ethyl acetate (2×50 mL). The combined organic layers were washed with brine (100 mL), dried over anhydrous sodium sulfate, filtered, and concentrated under reduced pressure. Purification by silica gel chromatography (Gradient: 0-30% EtOAc in heptane) afforded 6-chloro-3-[2-(1-methylcyclopropyl)ethynyl]pyrazin-2-amine (2.23 g, 82%) as a yellow solid. ESI-MS m/z calc. 207.05632, found 208.2 (M+1)⁺; Retention time: 1.76 minutes; LC method I.

### Step 3: 3-Chloro-5-methyl-6-(1-methylcyclopropyl)pyrrolo[2,3-b]pyrazine

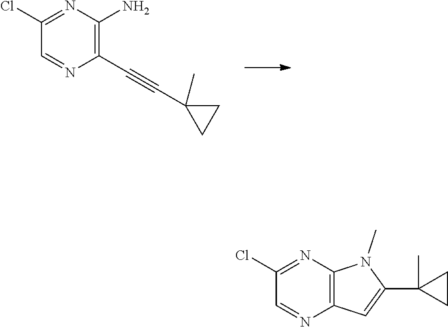

To a solution of 6-chloro-3-[2-(1-methylcyclopropyl)ethynyl]pyrazin-2-amine (2.23 g, 9.5682 mmol) in DMF (30 mL) was added potassium tert-butoxide (3.3 g, 29.409 mmol). The reaction mixture was heated at 80° C. for 50 min then the reaction was allowed to cool down to room temperature before Mel (2.7360 g, 1.2 mL, 19.276 mmol) was added dropwise. The reaction was stirred at room temperature for 1 hour then potassium tert-butoxide (1.02 g, 1.1308 mL, 9.0899 mmol) and Mel (2.2800 g, 1 mL, 16.063 mmol) were again added. The reaction mixture was stirred at room temperature for 18 hours then was diluted with H₂O (100 mL), ethyl acetate (50 mL) and brine (100 mL). The phases were separated, and the aqueous layer was extracted with ethyl acetate (3×50 mL). The combined organic layers were washed with a 50% solution of brine (4×100 mL), dried over anhydrous sodium sulfate, filtered, and concentrated under reduced pressure. The residue was diluted with EtOAc (30 mL) and activated carbon (1.5 g was added). The suspension was stirred for 10 minutes then the solution was filtered on a silica pad and washed with EtOAc (200 mL). The filtrate was concentrated under reduced pressure to afford 3-chloro-5-methyl-6-(1-methylcyclopropyl)pyrrolo[2,3-b]pyrazine (1.88 g, 82%) as a brown solid. ESI-MS m/z calc. 221.07198, found 222.2 (M+1)⁺; Retention time: 1.88 minutes; LC method I.

### Step 4: Methyl 5-methyl-6-(1-methylcyclopropyl)pyrrolo[2,3-b]pyrazine-3-carboxylate

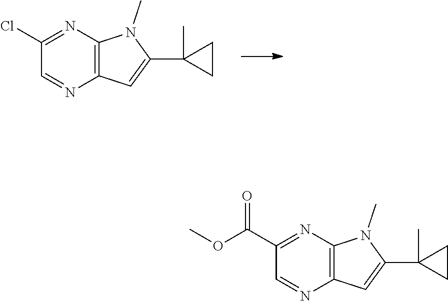 [SMILES: Cn1c(C2(C)CC2)cc2ncc(Cl)nc21 ; COC(=O)c1cnc2cc(C3(C)CC3)n(C)c2n1]

To a solution of 3-chloro-5-methyl-6-(1-methylcyclopropyl)pyrrolo[2,3-b]pyrazine (4.56 g, 19.788 mmol) in methanol (40 mL) was added triethylamine (5.8080 g, 8 mL, 57.397 mmol). The solution was sparged with nitrogen for 15 minutes then 1,1′-bis(diphenylphosphino)ferrocene palladium(II) chloride, complex with dichloromethane (872 mg, 1.0678 mmol) was added. The suspension was sparged with nitrogen for 5 minutes then was stirred at 100° C. under 50 psi of carbon monoxide for 18 hours. The mixture was allowed to cool down to room temperature and was filtered through Celite, washed with EtOAc (2×25 mL) and the filtrate was concentrated in vacuo. Purification by silica gel chromatography (Gradient: 0-50% EtOAc in heptane) afforded methyl 5-methyl-6-(1-methylcyclopropyl)pyrrolo[2,3-b]pyrazine-3-carboxylate (4.24 g, 85%) as a light yellow solid. ¹H NMR (400 MHz, CDCl₃) δ 9.17 (s, 1H), 6.52 (s, 1H), 4.04 (s, 3H), 4.02 (s, 3H), 1.49 (s, 3H), 1.07-1.00 (m, 2H), 0.95-0.89 (m, 2H). ESI-MS m/z calc. 245.11642, found 246.2 (M+1)⁺; Retention time: 1.66 minutes; LC method I.

### Step 5: [5-Methyl-6-(1-methylcyclopropyl)pyrrolo[2,3-b]pyrazin-3-yl]methanol

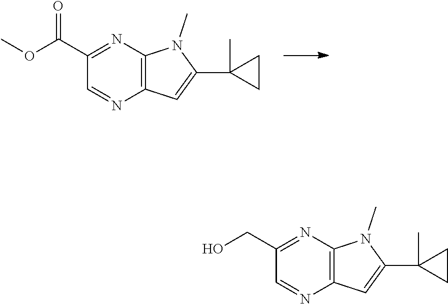 [SMILES: COC(=O)c1cnc2cc(C3(C)CC3)n(C)c2n1 ; Cn1c(C2(C)CC2)cc2ncc(CO)nc21]

A solution of LAH pellets (2.78 g, 73.246 mmol) in THE (85 mL) at 0° C. was stirred for 1 hour then a solution of methyl 5-methyl-6-(1-methylcyclopropyl)pyrrolo[2,3-b]pyrazine-3-carboxylate (4.24 g, 16.889 mmol) in THE (85 mL) was added dropwise over 20 minutes. The reaction mixture was stirred at that temperature for 1 hour then was stirred at room temperature for 1 hour. To the reaction mixture was slowly added water (2.5 mL) and a 15% aqueous sodium hydroxide solution (2.5 mL). The reaction mixture was diluted with EtOAc (100 mL) and anhydrous sodium sulfate (5 g) was added. The suspension was stirred at room temperature for 2 hours and 30 minutes and filtered through Celite then washed with EtOAc (2×100 mL). The filtrate was concentrated in vacuo to afford crude [5-methyl-6-(1-methylcyclopropyl)pyrrolo[2,3-b]pyrazin-3-yl]methanol (3.61 g, 58%) as an orange oil that was used in the following step without further purification. ¹H NMR (400 MHz, CDCl₃) δ 8.36 (s, 1H), 6.45-6.37 (m, 1H), 4.89 (s, 2H), 3.96-3.89 (m, 3H), 1.47 (s, 3H), 1.01-0.95 (m, 2H), 0.91-0.84 (m, 2H), (1H missing, labile proton). ESI-MS m/z calc. 217.1215, found 218.2 (M+1)⁺; Retention time: 1.43 minutes; LC method I.

### Step 6: 5-Methyl-6-(1-methylcyclopropyl)pyrrolo[2,3-b]pyrazine-3-carbaldehyde

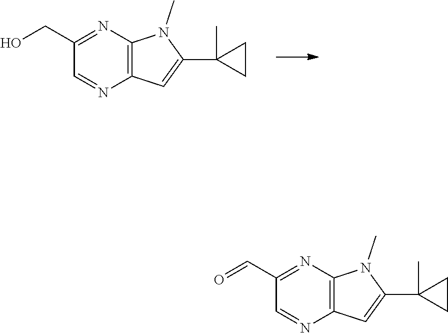 [SMILES: Cn1c(C2(C)CC2)cc2ncc(CO)nc21 ; Cn1c(C2(C)CC2)cc2ncc(C=O)nc21]

To a solution of [5-methyl-6-(1-methylcyclopropyl)pyrrolo[2,3-b]pyrazin-3-yl]methanol (3.61 g, 9.8696 mmol) in THF (500 mL) was added activated MnO₂ (33.86 g, 389.48 mmol) at room temperature. The suspension was stirred for 16 hours and was then filtered through Celite, washed with EtOAc (500 mL), and the filtrate was concentrated in vacuo. The crude residue was purified by silica gel chromatography (Gradient: 0-40% EtOAc in heptane) then by reverse phase chromatography (Column: C₁₈Aq. Gradient 0-100% MeCN in water). The purest fractions were combined and concentrated in vacuo to afford 5-methyl-6-(1-methylcyclopropyl)pyrrolo[2,3-b]pyrazine-3-carbaldehyde (797 mg, 37%) as a yellow solid. ¹H NMR (400 MHz, CDCl₃) δ 10.17 (s, 1H), 9.04 (s, 1H), 6.56 (s, 1H), 4.03 (s, 3H), 1.51 (s, 3H), 1.09-1.01 (m, 2H), 0.99-0.91 (m, 2H). ESI-MS m/z calc. 215.1059, found 216.2 (M+1)⁺; Retention time: 3.6 minutes. Mixed fractions containing the product were combined and concentrated in vacuo. Purification by reverse phase chromatography (Column: C₁₈Aq. Gradient: 5-40% MeCN in water) afforded a second batch of 5-methyl-6-(1-methylcyclopropyl)pyrrolo[2,3-b]pyrazine-3-carbaldehyde (220 mg, 10%) as a yellow solid. ¹H NMR (400 MHz, CDCl₃) δ 10.17 (s, 1H), 9.04 (s, 1H), 6.56 (s, 1H), 4.03 (s, 3H), 1.51 (s, 3H), 1.09-1.02 (m, 2H), 0.99-0.92 (m, 2H). ESI-MS m/z calc. 215.1059, found 216.2 (M+1)⁺; Retention time: 3.6 minutes. LC method J.

### Step 7: 3-[[4-[(2R)-3-(1-Bicyclo[1.1.1]]pentanyl)-2-[[5-methyl-6-(1-methylcyclopropyl)pyrrolo[2,3-b]pyrazin-3-yl]methylamino]propoxy]-6-(2,6-dimethylphenyl)pyrimidin-2-yl]sulfamoyl]benzoic acid

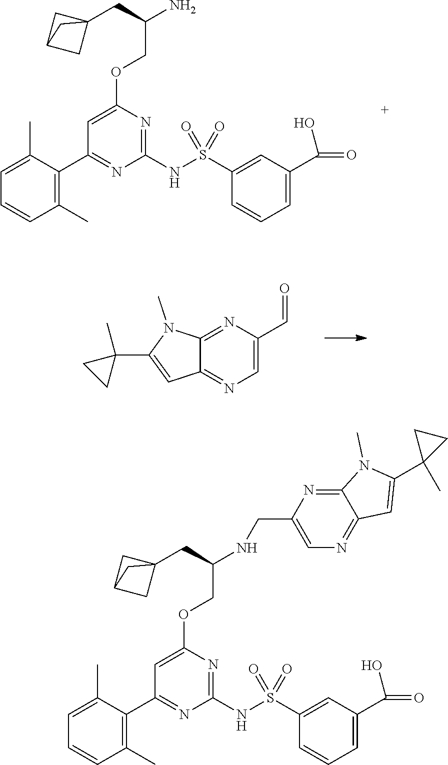 [SMILES: Cc1cccc(C)c1-c1cc(OC[C@H](N)CC23CC(C2)C3)nc(NS(=O)(=O)c2cccc(C(=O)O)c2)n1 ; Cn1c(C2(C)CC2)cc2ncc(C=O)nc21 ; Cc1cccc(C)c1-c1cc(OC[C@@H](CC23CC(C2)C3)NCc2cnc3cc(C4(C)CC4)n(C)c3n2)nc(NS(=O)(=O)c2cccc(C(=O)O)c2)n1]

3-[[4-[(2R)-2-Amino-3-(1-bicyclo[1.1.1]pentanyl)propoxy]-6-(2,6-dimethylphenyl)pyrimidin-2-yl]sulfamoyl]benzoic acid (hydrochloride salt) (60 mg, 0.09659 mmol) was combined with 5-methyl-6-(1-methylcyclopropyl)pyrrolo[2,3-b]pyrazine-3-carbaldehyde (23 mg, 0.1069 mmol) in DCM. Acetic acid (10 μL, 0.1758 mmol) was added and the reaction mixture was cooled to 0° C. in an ice water bath. DIPEA (45 μL, 0.2584 mmol) was added and after stirring at this temperature for 20 minutes sodium triacetoxyborohydride (approximately 102.4 mg, 0.4830 mmol) was added. Stirring at 0° C. was continued for 2 hours. The reaction mixture was then quenched with 0.2 mL 3 M HCl, diluted with methanol and DMSO until the reaction mixture became homogeneous, filtered, and purified by preparative HPLC (1-99% ACN in water, HCl modifier, 15 minute run) to give 3-[[4-[(2R)-3-(1-bicyclo[1.1.1]pentanyl)-2-[[5-methyl-6-(1-methylcyclopropyl)pyrrolo[2,3-b]pyrazin-3-yl]methylamino]propoxy]-6-(2,6-dimethylphenyl)pyrimidin-2-yl]sulfamoyl]benzoic acid (hydrochloride salt) (52.2 mg, 71%). ESI-MS m/z calc. 721.3046, found 722.8 (M+1)⁺; Retention time: 0.59 minutes; LC method B.

### Step 8: (11R)-11-(1-Bicyclo[1.1.1]pentanylmethyl)-6-(2,6-dimethylphenyl)-12-[[5-methyl-6-(1-methylcyclopropyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-227

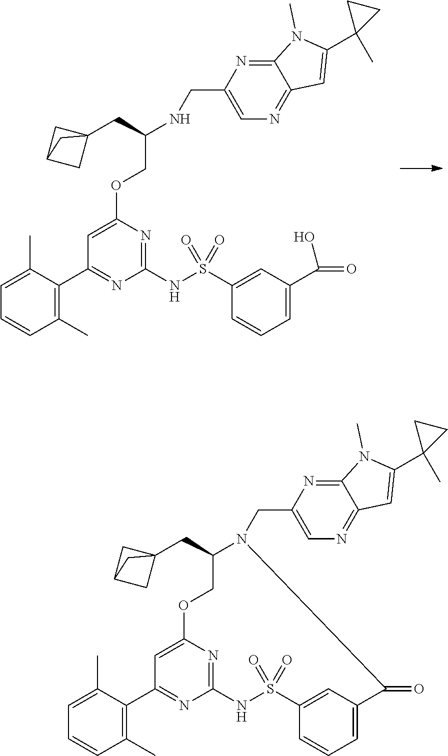 [SMILES: Cc1cccc(C)c1-c1cc(OC[C@@H](CC23CC(C2)C3)NCc2cnc3cc(C4(C)CC4)n(C)c3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3cc(C4(C)CC4)n(C)c3n1)[C@H](CC13CC(C1)C3)CO2]

3-[[4-[(2R)-3-(1-Bicyclo[1.1.1]pentanyl)-2-[[5-methyl-6-(1-methylcyclopropyl) pyrrolo[2,3-b]pyrazin-3-yl]methylamino]propoxy]-6-(2,6-dimethylphenyl)pyrimidin-2-yl]sulfamoyl]benzoic acid (hydrochloride salt) (52.2 mg, 0.06884 mmol) was combined with CDMT (15 mg, 0.08543 mmol) in DMF. N-methylmorpholine (50 μL, 0.4548 mmol) was added by syringe and the reaction was stirred at room temperature for the indicated time. Volatiles were then removed by rotary evaporation under reduced pressure, and the resulting residue was dissolved in DMSO/methanol, filtered, and purified by preparative HPLC (1-99% ACN in water, HCl modifier) to give (11R)-11-(1-bicyclo[1.1.1]pentanylmethyl)-6-(2,6-dimethyl phenyl)-12-[[5-methyl-6-(1-methylcyclopropyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (32.1 mg, 66%). ESI-MS m/z calc. 703.29407, found 704.7 (M+1)⁺; Retention time: 1.87 minutes; LC method A. ¹H NMR (400 MHz, Chloroform-d) δ 9.02 (t, J=1.8 Hz, 1H), 8.48 (s, 1H), 8.08 (d, J=7.9 Hz, 1H), 7.92 (dt, J=7.7, 1.4 Hz, 1H), 7.68 (t, J=7.8 Hz, 1H), 7.21 (t, J=7.6 Hz, 1H), 7.07 (d, J=7.6 Hz, 2H), 6.47 (s, 1H), 6.22 (s, 1H), 5.59 (dd, J=11.0, 3.6 Hz, 1H), 5.42 (d, J=15.9 Hz, 1H), 4.30 (d, J=15.9 Hz, 1H), 4.09 (tt, J=10.4, 3.4 Hz, 1H), 4.05-3.92 (m, 4H), 2.46 (s, 1H), 2.05 (s, 6H), 1.97 (dd, J=15.5, 10.5 Hz, 1H), 1.83 (dd, J=15.5, 3.3 Hz, 1H), 1.63-1.57 (m, 6H), 1.45 (s, 3H), 1.02-0.94 (m, 2H), 0.89-0.83 (m, 2H).

### Example 98: Preparation of Compound I-234

### Step 1: 5-Chloro-6-fluoro-2-(1-methylcyclopropyl)furo[3,2-b]pyridine

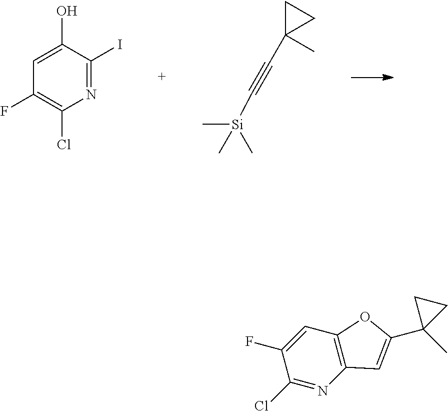

To a pressure vial containing 6-chloro-5-fluoro-2-iodo-pyridin-3-ol (5.4 g, 18.036 mmol), bis(triphenylphosphine)palladium(II) dichloride (500 mg, 0.7124 mmol), CuI (350 mg, 1.8378 mmol), KF (2.1 g, 36.147 mmol) in DMF (60 mL) and TEA (17 mL), trimethyl-[2-(1-methylcyclopropyl)ethynyl]silane (4 g, 22.323 mmol) was added. The reaction was stirred at 80° C. for 12 hours. The reaction mixture was allowed to cool, diluted with ethyl acetate (300 mL) and washed with water (2×300 mL). The organic layer was washed with brine (100 mL), dried with sodium sulfate, filtered and concentrated under vacuo. The residue was purified by silica gel chromatography in 0-30% ethyl acetate in hexanes to give 5-chloro-6-fluoro-2-(1-methylcyclopropyl)furo[3,2-b]pyridine (2.4 g, 53%) as white solid. ¹H NMR (500 MHz, CDCl₃) δ 7.45 (d, J=7.9 Hz, 1H), 6.51 (s, 1H), 1.50 (s, 3H), 1.28-1.24 (m, 2H), 0.93-0.87 (m, 2H). ESI-MS m/z calc. 225.03568, found 226.0 (M+1)⁺; Retention time: 5.68 minutes; LC method D.

### Step 2: Methyl 6-fluoro-2-(1-methylcyclopropyl)furo[3,2-b]pyridine-5-carboxylate

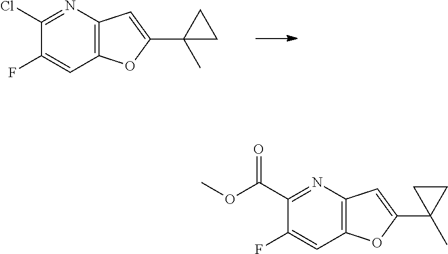 [SMILES: CC1(c2cc3nc(Cl)c(F)cc3o2)CC1 ; COC(=O)c1nc2cc(C3(C)CC3)oc2cc1F]

A mixture of 5-chloro-6-fluoro-2-(1-methylcyclopropyl)furo[3,2-b]pyridine (2.4 g, 9.5725 mmol), Pd(dppf)₂·DCM (350 mg, 0.4286 mmol) and TEA (5.0820 g, 7 mL, 50.222 mmol) in MeOH (50 mL) in a steel bomb equipped with mechanical stirrer was purged with carbon monoxide three times. The reaction mixture was heated to 60° C. with 100 psi of carbon monoxide and stirred for 6 h. The reaction mixture was allowed to cool to rt. The reaction mixture was filtered through a pad of zeolite, washed with ethyl acetate, filtrate was concentrated in vacuo. The residue was purified by silica gel chromatography in 10-50% ethyl acetate in hexanes to give methyl 6-fluoro-2-(1-methylcyclopropyl)furo[3,2-b]pyridine-5-carboxylate (1.9 g, 72%) as an orange solid. ¹H NMR (500 MHz, CDCl₃) δ 7.44 (d, J=10.0 Hz, 1H), 6.65 (s, 1H), 4.02 (s, 3H), 1.51 (s, 3H), 1.33-1.27 (m, 2H), 0.97-0.90 (m, 2H). ESI-MS m/z calc. 249.08012, found 250.0 (M+1)⁺; Retention time: 4.51 minutes; LC method D.

### Step 3: 6-Fluoro-2-(1-methylcyclopropyl)furo[3,2-b]pyridine-5-carbaldehyde

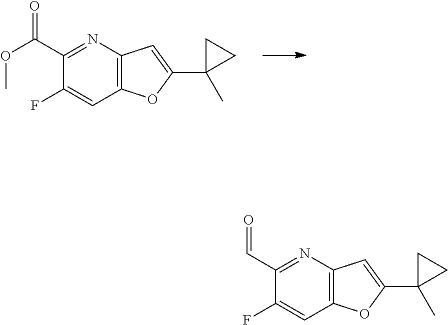 [SMILES: COC(=O)c1nc2cc(C3(C)CC3)oc2cc1F ; CC1(c2cc3nc(C=O)c(F)cc3o2)CC1]

To a stirred solution of methyl 6-fluoro-2-(1-methylcyclopropyl)furo[3,2-b]pyridine-5-carboxylate (1.9 g, 6.8609 mmol) in DCM (60 mL) was added DIBAL (14 mL of 1 M, 14.000 mmol) in DCM at −78° C. The reaction mixture was stirred for 2 h, quenched with a mixture of MeOH (3 mL) and water (3 mL) at −78° C. and the reaction was allowed to warm to rt. DCM (100 mL) was added and the solid was filtered. The cake was washed with DCM (3×50 mL). The organic layer from the filtrate was separated, washed with brine (2×50 mL), dried over sodium sulfate, filtered, and concentrated in vacuo. The residue was purified by silica gel chromatography, loaded with DCM, eluted with 20% ethyl acetate in hexanes. The desired product fractions were combined, concentrated in vacuo and dried overnight to give 6-fluoro-2-(1-methylcyclopropyl)furo[3,2-b]pyridine-5-carbaldehyde (1.193 g, 78%) as an off-white solid. ¹H NMR (500 MHz, CDCl₃) δ 10.28 (s, 1H), 7.45 (d, J=10.0 Hz, 1H), 6.66 (s, 1H), 1.53 (s, 3H), 1.34-1.27 (m, 2H), 0.97-0.91 (m, 2H). ESI-MS m/z calc. 219.06955, found 220.0 (M+1)⁺; Retention time: 2.2 minutes; LC method H.

### Step 4: 3-[[4-(2,6-Dimethylphenyl)-6-[(2R)-2-[[6-fluoro-2-(1-methylcyclopropyl)furo[3,2-b]pyridin-5-yl]methylamino]-3-(1-methylcyclopropyl)propoxy]pyrimidin-2-yl]sulfamoyl]benzoic acid

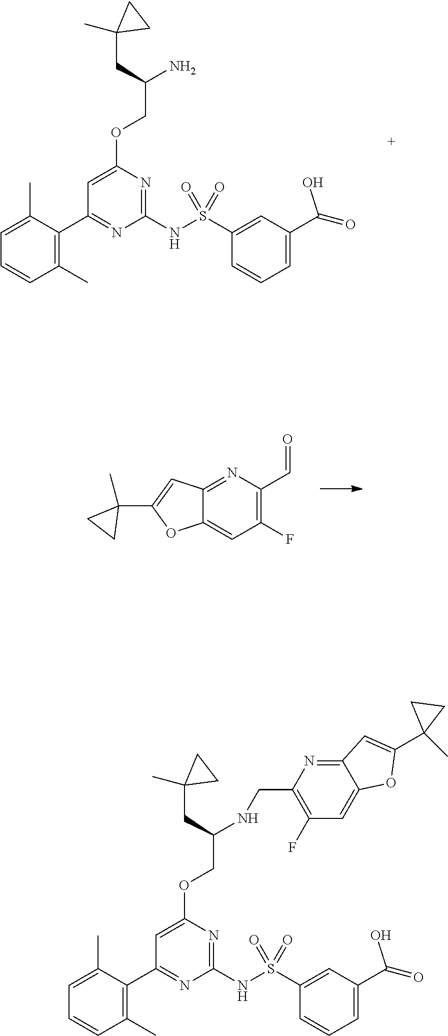 [SMILES: Cc1cccc(C)c1-c1cc(OC[C@H](N)CC2(C)CC2)nc(NS(=O)(=O)c2cccc(C(=O)O)c2)n1 ; CC1(c2cc3nc(C=O)c(F)cc3o2)CC1 ; Cc1cccc(C)c1-c1cc(OC[C@@H](CC2(C)CC2)NCc2nc3cc(C4(C)CC4)oc3cc2F)nc(NS(=O)(=O)c2cccc(C(=O)O)c2)n1]

3-[[4-[(2R)-2-Amino-3-(1-methylcyclopropyl)propoxy]-6-(2,6-dimethylphenyl)pyrimidin-2-yl]sulfamoyl]benzoic acid (hydrochloride salt) (60 mg, 0.1097 mmol) was combined with 6-fluoro-2-(1-methylcyclopropyl)furo[3,2-b]pyridine-5-carbaldehyde (26.5 mg, 0.1209 mmol) in DCM. acetic acid (10 μL, 0.1758 mmol) was added and the reaction mixture was cooled to 0° C. in an ice water bath. DIPEA (50 μL, 0.2871 mmol) was added and after stirring at this temperature for 20 minutes sodium triacetoxyborohydride (120 mg, 0.5662 mmol) was added. Stirring at 0° C. was continued for 2 hours. The reaction mixture was then quenched with 0.2 mL 3M HCl, diluted with methanol and DMSO until the reaction mixture became homogeneous, filtered, and purified by preparative HPLC (1-99% ACN in water, HCl modifier, 15 minute run) to give 3-[[4-(2,6-dimethylphenyl)-6-[(2R)-2-[[6-fluoro-2-(1-methylcyclopropyl)furo[3,2-b]pyridin-5-yl]methylamino]-3-(1-methylcyclopropyl)propoxy]pyrimidin-2-yl]sulfamoyl]benzoic acid (hydrochloride salt) (40.3 mg, 49%). ESI-MS m/z calc. 713.2683, found 714.7 (M+1)⁺; Retention time: 0.61 minutes; LC method B.

### Step 5: (11R)-6-(2,6-Dimethylphenyl)-12-[[6-fluoro-2-(1-methylcyclopropyl)furo[3,2-b]pyridin-5-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-234

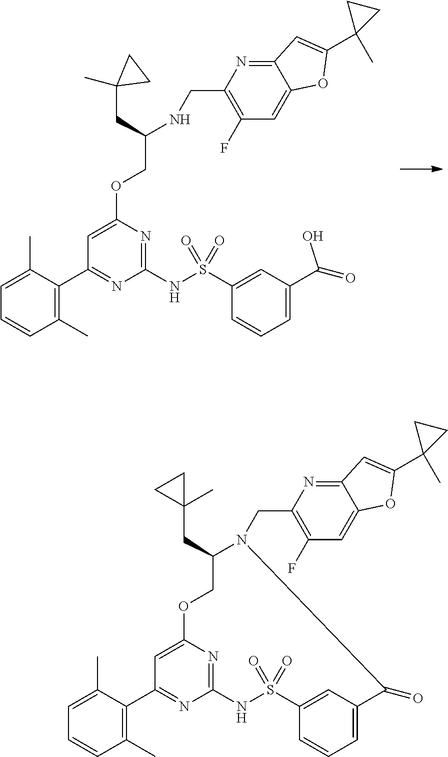 [SMILES: Cc1cccc(C)c1-c1cc(OC[C@@H](CC2(C)CC2)NCc2nc3cc(C4(C)CC4)oc3cc2F)nc(NS(=O)(=O)c2cccc(C(=O)O)c2)n1 ; Cc1cccc(C)c1-c1cc2nc(n1)NS(=O)(=O)c1cccc(c1)C(=O)N(Cc1nc3cc(C4(C)CC4)oc3cc1F)[C@H](CC1(C)CC1)CO2]

3-[[4-(2,6-Dimethylphenyl)-6-[(2R)-2-[[6-fluoro-2-(1-methylcyclopropyl)furo[3,2-b]pyridin-5-yl]methylamino]-3-(1-methylcyclopropyl)propoxy]pyrimidin-2-yl]sulfamoyl]benzoic acid (hydrochloride salt) (40.3 mg, 0.05371 mmol) was combined with CDMT (11.5 mg, 0.06550 mmol) in DMF. N-methylmorpholine (40 μL, 0.3638 mmol) was added by syringe and the reaction was stirred at room temperature for the indicated time. Volatiles were then removed by rotary evaporation under reduced pressure, and the resulting residue was dissolved in DMSO/methanol, filtered, and purified by preparative HPLC (1-99% ACN in water, HCl modifier) to give (11R)-6-(2,6-dimethylphenyl)-12-[[6-fluoro-2-(1-methylcyclopropyl)furo[3,2-b]pyridin-5-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (22.2 mg, 57%). ESI-MS m/z calc. 695.25775, found 696.8 (M+1)⁺; Retention time: 2.17 minutes; LC method A. ¹H NMR (400 MHz, Chloroform-d) δ 9.12 (t, J=1.8 Hz, 1H), 8.08 (dt, J=7.9, 1.5 Hz, 1H), 7.87 (dt, J=7.5, 1.4 Hz, 1H), 7.64 (t, J=7.8 Hz, 1H), 7.36 (dd, J=9.6, 1.0 Hz, 1H), 7.20 (t, J=7.6 Hz, 1H), 7.06 (d, J=7.6 Hz, 2H), 6.66 (s, 1H), 6.22 (s, 1H), 5.58 (dd, J=11.4, 4.3 Hz, 1H), 5.47 (dd, J=16.1, 1.7 Hz, 1H), 4.41-4.30 (m, 1H), 4.15 (dd, J=16.1, 1.7 Hz, 1H), 3.85 (t, J=11.6 Hz, 1H), 2.02 (s, 6H), 1.80 (d, J=14.9 Hz, 1H), 1.49 (s, 3H), 1.47-1.43 (m, 1H), 1.24-1.20 (m, 2H), 0.87-0.82 (m, 2H), 0.55 (s, 3H), 0.45-0.37 (m, 1H), 0.30-0.24 (m, 1H), 0.17-0.11 (m, 1H), 0.07-0.01 (m, 1H).

### Example 99: Preparation of Compound I-237

### Step 1: (11R)-12-[(7-Amino-6-isopropyl-furo[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-237

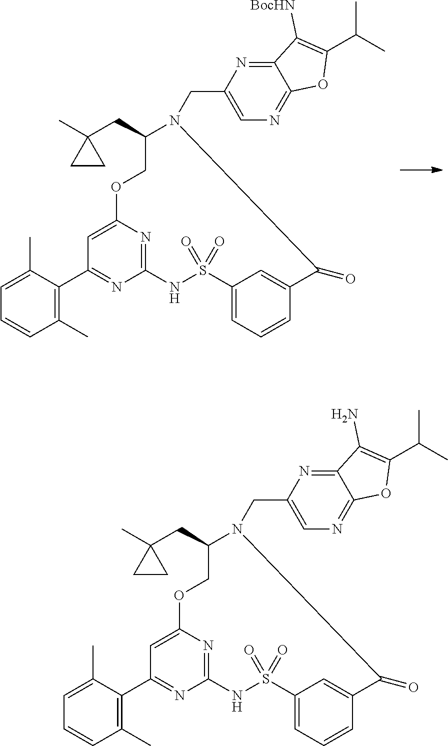 [SMILES: Cc1cccc(C)c1-c1cc2nc(n1)NS(=O)(=O)c1cccc(c1)C(=O)N(Cc1cnc3oc(C(C)C)c(NC(=O)OC(C)(C)C)c3n1)[C@H](CC1(C)CC1)CO2 ; Cc1cccc(C)c1-c1cc2nc(n1)NS(=O)(=O)c1cccc(c1)C(=O)N(Cc1cnc3oc(C(C)C)c(N)c3n1)[C@H](CC1(C)CC1)CO2]

To a solution of tert-butyl N-[2-[[(11R)-6-(2,6-dimethylphenyl)-11-[(1-methylcyclopropyl)methyl]-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-6-isopropyl-furo[2,3-b]pyrazin-7-yl]carbamate (11.0 mg, 0.01322 mmol) in dichloromethane (0.024 mL) was added trifluoroacetic Acid (1) (12.3 μL, 0.1597 mmol) and the resulting solution was stirred at room temperature overnight. The crude reaction mixture was concentrated under reduced pressure. The residue was dissolved in DCM and washed with saturated NaHCO₃ (3×). The organic layer was dried over MgSO₄ and concentrated under reduced pressure. The crude product was diluted with DMSO (1 mL) and MeOH (1 mL), filtered, and purified by reverse phase HPLC (1-99% acetonitrile/5 mM aqueous HCl over 25 min) to give (11R)-12-[(7-amino-6-isopropyl-furo[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (1.7 mg, 18%), ¹H NMR (400 MHz, CDCl₃) δ 9.01 (t, J=1.9 Hz, 1H), 8.29 (s, 1H), 8.08 (d, J=8.1 Hz, 1H), 7.87 (dt, J=7.7, 1.4 Hz, 1H), 7.64 (t, J=7.8 Hz, 1H), 7.21 (t, J=7.6 Hz, 1H), 7.07 (d, J=7.6 Hz, 2H), 6.23 (s, 1H), 5.67 (dd, J=11.4, 4.2 Hz, 1H), 5.36 (d, J=15.6 Hz, 1H), 4.43-4.34 (m, 1H), 4.21 (d, J=15.6 Hz, 1H), 4.07 (t, J=11.5 Hz, 1H), 3.23 (hept, J=7.0 Hz, 1H), 2.03 (s, 6H), 1.91-1.83 (m, 2H), 1.48-1.42 (m, 2H), 1.39 (d, J=3.0 Hz, 3H), 1.37 (d, J=3.0 Hz, 3H), 0.56 (s, 3H), 0.43-0.36 (m, 1H), 0.32-0.25 (m, 1H), 0.19-0.13 (m, 1H), 0.06-0.02 (m, 1H). ESI-MS m/z calc. 681.2733, found 682.8 (M+1)⁺; Retention time: 1.79 minutes. LC method A.

### Example 100: Preparation of (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol

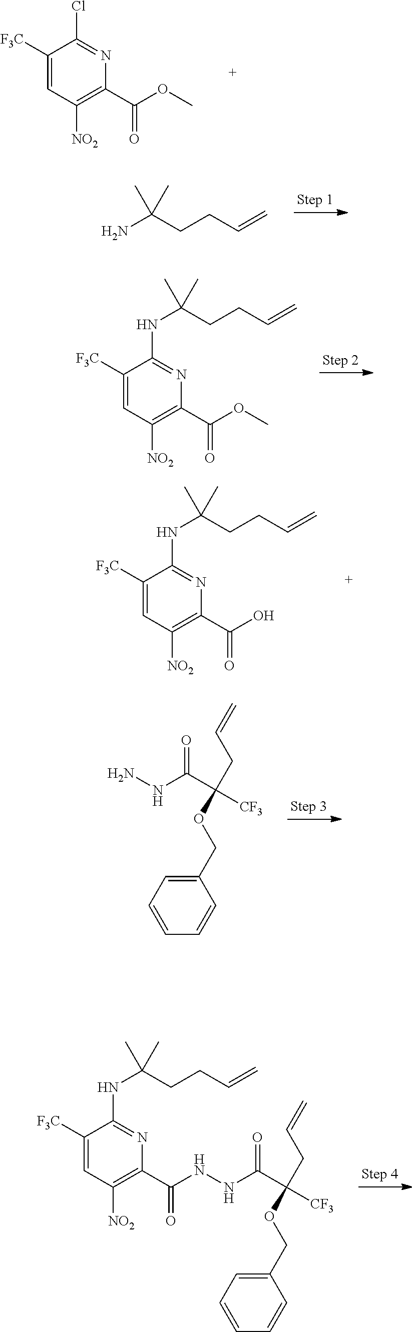

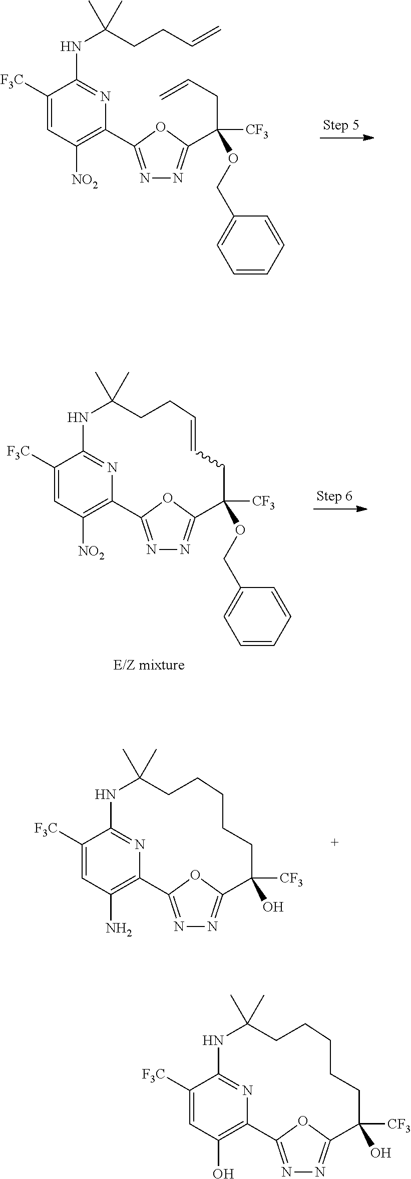 [SMILES: C=CCCC(C)(C)Nc1nc(-c2nnc([C@@](CC=C)(OCc3ccccc3)C(F)(F)F)o2)c([N+](=O)[O-])cc1C(F)(F)F ; CC1(C)CCC=CC[C@](OCc2ccccc2)(C(F)(F)F)C2=NN=C(O2)c2nc(c(C(F)(F)F)cc2[N+](=O)[O-])N1 ; CC1(C)CCCCC[C@](O)(C(F)(F)F)C2=NN=C(O2)c2nc(c(C(F)(F)F)cc2N)N1 ; CC1(C)CCCCC[C@](O)(C(F)(F)F)C2=NN=C(O2)c2nc(c(C(F)(F)F)cc2O)N1]

### Step 1: Methyl 6-(1,1-dimethylpent-4-enylamino)-3-nitro-5-(trifluoromethyl)pyridine-2-carboxylate

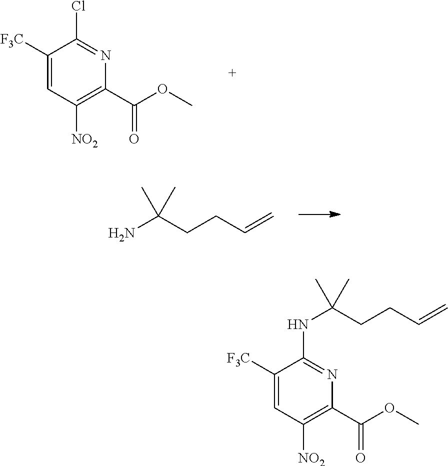

2-Methylhex-5-en-2-amine (hydrochloride salt) (69.4 g, 463.7 mmol) was suspended in acetonitrile (960 mL) and treated with DIEA (220 mL, 1.263 mol). To the formed brown solution was added methyl 6-chloro-3-nitro-5-(trifluoromethyl)pyridine-2-carboxylate (120 g, 421.7 mmol) in one portion. The orange solution was slowly heated to 65° C. over 2.5 h (Note: reaction shows exotherm on heating). The deep orange solution was evaporated at 40° C. and to the residue was added with MTBE (1 L) and water (1 L) and the layers were separated. The deep orange organic phase was washed with a 1:1 solution of saturated aqueous NH₄Cl/water mixture (2×600 mL), once with brine (400 mL) and the organic phase was dried, filtered and evaporated to give methyl 6-(1,1-dimethylpent-4-enylamino)-3-nitro-5-(trifluoromethyl)pyridine-2-carboxylate (152.7 g, 100%). ESI-MS m/z calc. 361.12494, found 362.0 (M+1)⁺; Retention time: 3.02 minutes (LC Method M).

### Step 2: 6-(1,1-Dimethylpent-4-enylamino)-3-nitro-5-(trifluoromethyl)pyridine-2-carboxylic acid

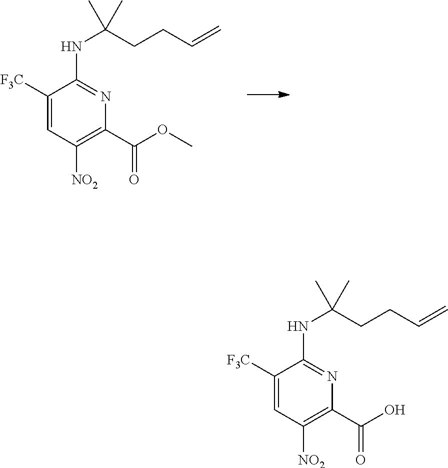 [SMILES: C=CCCC(C)(C)Nc1nc(C(=O)OC)c([N+](=O)[O-])cc1C(F)(F)F ; C=CCCC(C)(C)Nc1nc(C(=O)O)c([N+](=O)[O-])cc1C(F)(F)F]

Methyl 6-(1,1-dimethylpent-4-enylamino)-3-nitro-5-(trifluoromethyl)pyridine-2-carboxylate (152.4 g, 421.7 mmol) was dissolved in methanol (750 mL) and treated with NaOH (750 mL of 2 M, 1.500 mol) under stirring (added all at once giving a slight exotherm from 30° C. to 40° C.). The solution was stirred at room temperate for 18 h. The deep red solution was concentrated under reduced pressure at 42° C. and the resulting orange red solution was treated with toluene (1 L). The emulsion was stirred in an ice bath and acidified to pH=1 by addition of HCl (260 mL of 6 M, 1.560 mol) keeping the internal temperature below 15° C. The phases were separated and the organic phase was washed twice with water (2×500 mL) and once with brine (400 mL). The organic phase was dried over MgSO₄, filtered, evaporated and dried under vacuum to give 137 g of a deep orange mass of solid. This material was evaporated from acetonitrile (˜1 L, to remove residual toluene) and dissolved in acetonitrile (600 mL) and warmed to −60° C. To the deep red hot solution was added N-cyclohexylcyclohexanamine (79 mL, 396.5 mmol) under stirring (exotherm noted from 60° C. to 70° C.) and the hot solution was seeded. The material became a solid mass at an internal temperature of ˜60° C., which could be stirred magnetically after breaking up. The thick suspension was stirred in the cooling hot water bath overnight and then in an ice bath for 3 h. The solid was collected by filtration, washed with cold acetonitrile until the filtrate was colorless and dried over the weekend to give 6-(1,1-dimethylpent-4-enylamino)-3-nitro-5-(trifluoromethyl)pyridine-2-carboxylic acid (dicyclohexylamine salt) (172 g, 77%) as a yellow solid. This salt was suspended in MTBE (1 L) and treated with citric acid (1.2 L of 1 M, 1.200 mol). The mixture was stirred and the phases were separated. The organic phase was washed twice more with 1 M citric acid (2×400 mL) and 4 times with 0.5M KHSO₄ (4×400 mL). The organic phase was then washed once with brine (200 mL), dried, filtered and evaporated to give 6-(1,1-dimethylpent-4-enylamino)-3-nitro-5-(trifluoromethyl)pyridine-2-carboxylic acid (113.4 g, 77%) as a yellow orange oil, which crystallized upon standing. ¹H NMR (400 MHz, DMSO-d6) δ 14.21 (s, 1H), 8.46 (s, 1H), 6.20-6.00 (m, 1H), 5.82-5.57 (m, 1H), 5.13-4.74 (m, 2H), 1.97 (d, J=2.9 Hz, 4H), 1.45 (s, 6H) ppm. ESI-MS m/z calc. 347.10928, found 348.0 (M+1)⁺; Retention time: 2.49 minutes (LC Method M).

### Step 3: N′-[(2R)-2-Benzyloxy-2-(trifluoromethyl)pent-4-enoyl]-6-(1,1-dimethylpent-4-enylamino)-3-nitro-5-(trifluoromethyl)pyridine-2-carbohydrazide

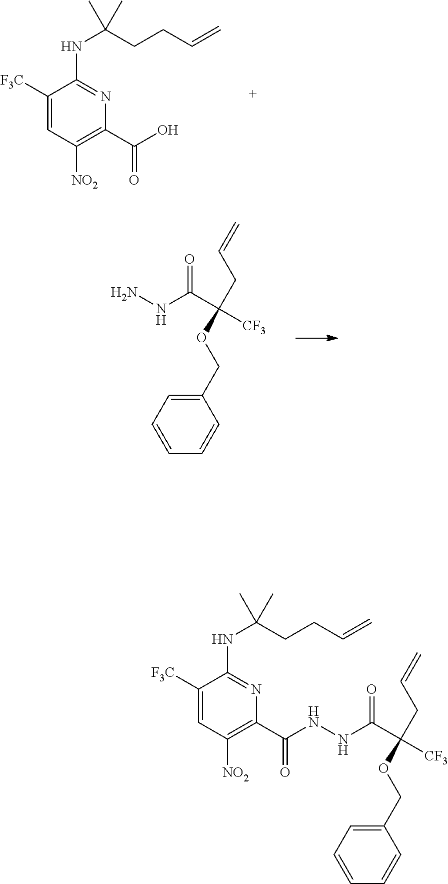 [SMILES: C=CCCC(C)(C)Nc1nc(C(=O)O)c([N+](=O)[O-])cc1C(F)(F)F ; C=CC[C@@](OCc1ccccc1)(C(=O)NN)C(F)(F)F ; C=CCCC(C)(C)Nc1nc(C(=O)NNC(=O)[C@@](CC=C)(OCc2ccccc2)C(F)(F)F)c([N+](=O)[O-])cc1C(F)(F)F]

6-(1,1-Dimethylpent-4-enylamino)-3-nitro-5-(trifluoromethyl)pyridine-2-carboxylic acid (100 g, 285.1 mmol) and (2R)-2-benzyloxy-2-(trifluoromethyl)pent-4-enehydrazide (86.3 g, 299.4 mmol) were dissolved in DMF (600 mL) and cooled in an ice bath. At an internal temperature of 3.1° C., HATU (114 g, 299.8 mmol) was added in one portion (no exotherm observed). Then, DIEA (100 mL, 574.1 mmol) was slowly added over 0.5 h (exothermic) keeping the internal temperate between 3 and 10° C. After the addition, the ice bath was removed and the reaction was stirred for another 0.5 h allowing it to warm to room temperature. The orange solution was added to a stirred solution of ice and water (3 L) and MTBE (1 L). The mixture was stirred for 10 minutes and the phases were separated. The organic phase was washed twice with water (2×1 L), 0.2 M KHSO₄ (3×1 L) and once with brine (250 mL). The organic phase was dried, filtered and evaporated to give as an orange mass, N-[(2R)-2-benzyloxy-2-(trifluoromethyl)pent-4-enoyl]-6-(1,1-dimethylpent-4-enylamino)-3-nitro-5-(trifluoromethyl)pyridine-2-carbohydrazide (181 g, quantitative yield). ESI-MS m/z calc. 617.2073, found 618.0 (M+1)⁺; Retention time: 3.25 minutes (LC Method M). This material was used directly in the next step.

### Step 4: 6-[5-[(1R)-1-Benzyloxy-1-(trifluoromethyl)but-3-enyl]-1,3,4-oxadiazol-2-yl]-N-(1,1-dimethylpent-4-enyl)-5-nitro-3-(trifluoromethyl)pyridin-2-amine

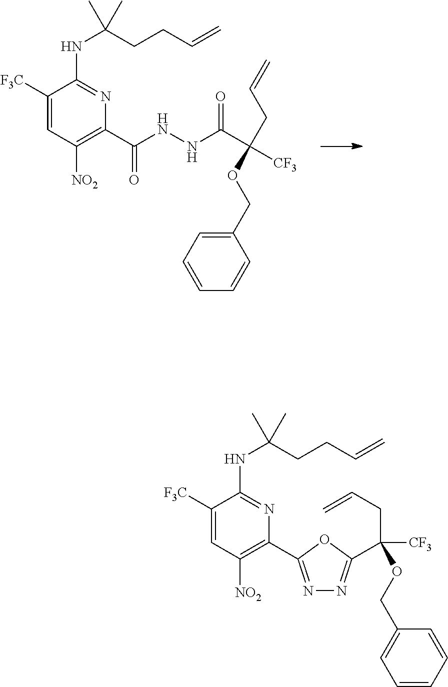 [SMILES: C=CCCC(C)(C)Nc1nc(C(=O)NNC(=O)[C@@](CC=C)(OCc2ccccc2)C(F)(F)F)c([N+](=O)[O-])cc1C(F)(F)F ; C=CCCC(C)(C)Nc1nc(-c2nnc([C@@](CC=C)(OCc3ccccc3)C(F)(F)F)o2)c([N+](=O)[O-])cc1C(F)(F)F]

N′-[(2R)-2-Benzyloxy-2-(trifluoromethyl)pent-4-enoyl]-6-(1,1-dimethylpent-4-enylamino)-3-nitro-5-(trifluoromethyl)pyridine-2-carbohydrazide (176.1 g, 285.2 mmol) was dissolved in acetonitrile (1.4 L) and heated to 55° C. The yellow orange solution was treated with DIEA (124 mL, 711.9 mmol) followed by portion-wise addition of tosyl chloride (54.4 g, 285.3 mmol) over 15 min (exothermic, internal temperature kept between 55° C. and 60° C. by removing the heating mantel and slow addition) and the reaction was stirred at 55-60° C. for 45 min. The reaction solution was concentrated under reduced pressure at 40° C. and the residue was extracted with MTBE/heptane 1:1 (1.4 L) and water (1.4 L). The organic phase was washed once more with water (1.5 L), twice with 0.2M KHSO₄ (2×1 L) and once with brine (0.5 L). The organic phase was dried, filtered and evaporated to give 172 g of an orange oil which was dissolved in 100 mL of toluene and 300 mL of heptane. The solution was loaded onto a 3 kg silica column (column volume=4800 mL, flow rate=900 mL/min). Eluted with 100% hexanes for 1 min, then programmed an initial gradient of 0% to 10% ethyl acetate in hexanes over 106 min (2 column volumes). The product started eluting at ˜4% ethyl acetate, so 4.3% ethyl acetate was held isocratically until the product finished eluting to give 6-[5-[(1R)-1-benzyloxy-1-(trifluoromethyl)but-3-enyl]-1,3,4-oxadiazol-2-yl]-N-(1,1-dimethylpent-4-enyl)-5-nitro-3-(trifluoromethyl)pyridin-2-amine (139.1 g, 80%). ¹H NMR (400 MHz, Chloroform-d) δ 8.51 (s, 1H), 7.40-7.27 (m, 5H), 6.03-5.87 (m, 1H), 5.80-5.66 (m, 1H), 5.58 (s, 1H), 5.31-5.16 (m, 2H), 5.03-4.95 (m, 1H), 4.95-4.89 (m, 1H), 4.81 (d, J=10.5 Hz, 1H), 4.64 (d, J=10.5 Hz, 1H), 3.28-3.13 (m, 2H), 2.08-1.99 (m, 2H), 1.99-1.89 (m, 2H), 1.47 (s, 6H) ppm. ESI-MS m/z calc. 599.1967, found 600.0 (M+1)⁺; Retention time: 3.71 minutes (LC Method M).

### Step 5: (6R)-6-Benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture

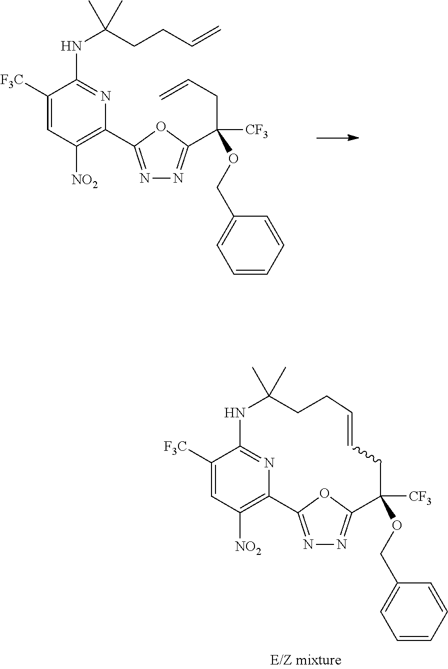 [SMILES: C=CCCC(C)(C)Nc1nc(-c2nnc([C@@](CC=C)(OCc3ccccc3)C(F)(F)F)o2)c([N+](=O)[O-])cc1C(F)(F)F ; CC1(C)CCC=CC[C@](OCc2ccccc2)(C(F)(F)F)C2=NN=C(O2)c2nc(c(C(F)(F)F)cc2[N+](=O)[O-])N1]

This reaction was run in three, 46.3 g batches in parallel, each in a 12 μL, 3-neck round bottom flask. The experimental below describes one of these batches.

Attached a sparging tube, reflux condenser with gas bubbler & overhead stirrer to a 12 L vessel placed in heat blanket. Dissolved 6-[5-[(1R)-1-benzyloxy-1-(trifluoromethyl)but-3-enyl]-1,3,4-oxadiazol-2-yl]-N-(1,1-dimethylpent-4-enyl)-5-nitro-3-(trifluoromethyl)pyridin-2-amine (46.3 g, 76.22 mmol) in DCE (8.23 L). Sparged the system with a heavy stream of nitrogen gas. Set the heat blanket for 50° C. When the vessel reached 53° C., added dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (Zhan catalyst-1B, 11.2 g, 15.26 mmol) all at once. Rinsed the catalyst container with DCE and added the rinse to the reaction. On completion of catalyst addition, increased the blanket temperature to 73° C. Once internal temperature reached 72° C., continued stirring for 2 h 28 min then decreased the heat blanket temperature to 45° C. After 2 h 27 min, internal temperature reached 50° C. After 15 min, added solid 2-sulfanylpyridine-3-carboxylic acid (12 g, 77.33 mmol) and triethylamine (11 mL, 78.92 mmol). Stirred for 12 h then allowed the mixture to cool to room temperature. Added 100 g of SiO₂ and 10 g of activated carbon (20-40 mesh, granular) to the reaction. Stirred for 1 h then filtered over Celite and evaporated the filtrate giving the crude product mixture. Combined the material from all three parallel reactions to give 71.2 g of crude product mixture. This material was purified on two separate 3 kg silica gel columns using a gradient from 100% hexanes to 10% ethyl acetate in hexanes over 110 min followed by a gradient from 10% ethyl acetate in hexanes to 100% ethyl acetate over 10 minutes. After combining the two separate purified batches, obtained (6R)-6-benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture) (51.88 g, 40%). ¹H NMR (400 MHz, DMSO-d6) δ 8.55 (d, J=0.8 Hz, 1H), 7.49-7.21 (m, 5H), 6.58 (s, 1H), 5.79 (dt, J=13.7, 6.5 Hz, 1H), 5.58 (ddd, J=15.0, 8.8, 5.6 Hz, 1H), 4.83 (d, J=11.1 Hz, 1H), 4.55 (d, J=11.1 Hz, 1H), 3.13 (dd, J=14.2, 5.4 Hz, 1H), 2.77 (dd, J=14.3, 8.8 Hz, 1H), 2.38-2.24 (m, 1H), 2.14-1.93 (m, 3H), 1.58-1.32 (m, 6H) ppm. ESI-MS m/z calc. 571.1654, found 572.1 (M+1)⁺; Retention times: 3.46 minutes and 3.49 minutes (LC Method M). Product formed as a 3:1 mixture of double bond isomers.

### Step 6: (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol

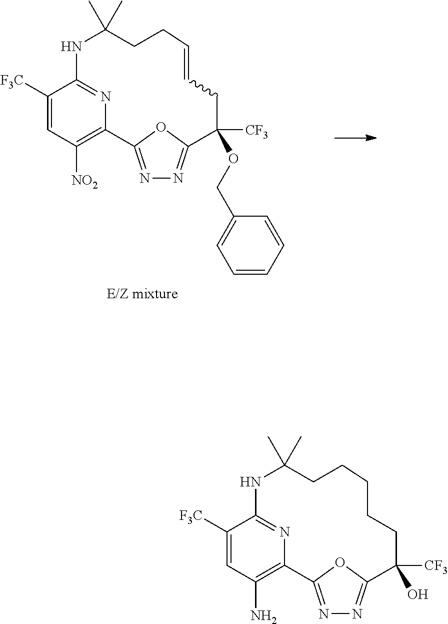 [SMILES: CC1(C)CCC=CC[C@](OCc2ccccc2)(C(F)(F)F)C2=NN=C(O2)c2nc(c(C(F)(F)F)cc2[N+](=O)[O-])N1 ; CC1(C)CCCCC[C@](O)(C(F)(F)F)C2=NN=C(O2)c2nc(c(C(F)(F)F)cc2N)N1]

(6R)-6-Benzyloxy-12,12-dimethyl-17-nitro-6,15-bis(trifluoromethyl)-19-oxa-3,4,13,18-tetrazatricyclo[12.3.1.12,5]nonadeca-1(18),2,4,8,14,16-hexaene (E/Z mixture) (50.8 g, 88.89 mmol) was dissolved in 250 mL of ethanol and partially concentrated by rotary evaporation with 28° C. water bath to remove any residual solvents then dissolved in further ethanol (720 mL) in a 5 L flask. Degassed the solution using 5 cycles of house vacuum with nitrogen gas backfill. Added dihydroxypalladium (15.2 g of 10% w/w, 10.824 mmol) to the substrate solution under nitrogen. Repeated house vacuum with hydrogen backfill for 6 cycles to replace nitrogen atmosphere with hydrogen. Finally kept the vessel under 1 atmosphere of hydrogen using a balloon. Stirred this mixture vigorously with a magnetic stirrer overnight then removed the hydrogen balloon. Filtered the mixture through 70 g of Celite on a medium-fritted funnel. Concentrated the green filtrate solution by rotary evaporation with a 28° C. water bath. Obtained 42.65 g of crude product as a yellow solid of which 41.5 g was purified by reverse-phase chromatography (dissolved in 125 mL of methanol and 2.55 mL DMF (2% DMF/methanol solution) and loaded onto a 3.8 kg C₁₈ column (column volume=3.3 μL, flow rate=375 mL/min). Programmed an initial gradient of 40% to 70% acetonitrile in water over 176 minutes (20 column volumes), then brought the eluent to 100% acetonitrile over the following ˜20 min). Mixed and pure fractions were isolated from the column. Pure fractions were concentrated to give as a yellow solid, (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol (28.17 g, 70%). This material was combined with several smaller batches (80 mg, 340 mg, 360 mg, 1.46 g and 1.63 g) made by similar methods as a solution in acetonitrile which was then concentrated to give a yellow solid. This solid was dissolved in dichloromethane and heptane was added then the solution was concentrated under vacuum in the dark at 40° C. overnight, which gave 31.95 g of (6R)-17-amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol. ¹H NMR (400 MHz, DMSO-d6) δ 7.61 (s, 1H), 7.59 (s, 1H), 5.96 (s, 2H), 4.64 (s, 1H), 2.90-2.71 (m, 1H), 2.30-2.15 (m, 1H), 2.15-1.98 (m, 1H), 1.91-1.74 (m, 1H), 1.73-1.57 (m, 1H), 1.56-1.38 (m, 5H), 1.36 (s, 3H), 1.31 (s, 3H). ESI-MS m/z calc. 453.15994, found 454.2 (M+1)⁺; Retention time: 3.03 minutes (LC Method M).

### Step 7: Solid form characterization of crystalline (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol Form A (neat

### A. X-Ray Powder Diffraction

The XRPD diffractogram for crystalline (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol Form A (neat) produced by Step 6 and recrystallized from EtOH was acquired using the General X-Ray Powder Diffraction (XRPD) Method. The XRPD diffractogram for crystalline (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol Form A (neat) is summarized below.

XRPD signals for crystalline (6R)-17-Amino-12,12-dimethyl-6,15-bis(trifluoromethyl)-19-oxa-3,4,13,18-tetrazatricyclo[12.3.1.12,5]nonadeca-1(18),2,4,14,16-pentaen-6-ol Form A (neat) include one or more of the following: 7.43±0.2 degrees 2-Theta, 8.44±0.2 degrees 2-Theta, 14.10±0.2 degrees 2-Theta, 14.57±0.2 degrees 2-Theta, 14.96±0.2 degrees 2-Theta, 16.94±0.2 degrees 2-Theta, 19.05±0.2 degrees 2-Theta, 19.97±0.2 degrees 2-Theta, 22.48±0.2 degrees 2-Theta, 25.56±0.2 degrees 2-Theta, and 25.75±0.2 degrees 2-Theta.

### Example 101: Preparation of 3-[[4-Chloro-6-(2,6-dimethylphenyl)-5-ethyl-pyrimidin-2-yl]sulfamoyl]benzoic acid

### Step 1: 2-Amino-5-ethyl-pyrimidine-4,6-diol

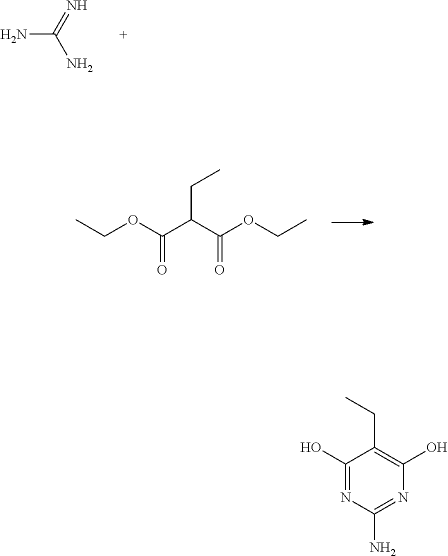

To a 2.0 L three neck flask was added ethanol (800 mL) and solid chunks of sodium metal (23 g, 1.000 mol) were added gradually and carefully under nitrogen flow and the mixture was stirred until completely dissolved. Once cooled back to room temperature guanidine (hydrochloride salt) (36.5 g, 382.1 mmol) and diethyl 2-ethylpropanedioate (56.22 g, 56 mL, 298.7 mmol) were successively added and the reaction was stirred with a mantle set at 80° C. (probe in the solution) for 17 hours. Once cooled to room temperature, the crude mixture was concentrated under reduced pressure to remove most of the ethanol. Water (400 mL) was added, the resulting solution was cooled in an ice bath and acidified to pH of 1-2 using concentrated HCl. The solids were filtered and washed with water (2×100 mL) followed by cold acetone (250 mL). The solid was dried in the fume hood overnight and transferred to a crystallization dish under high vacuum for three days to provide 2-amino-5-ethyl-pyrimidine-4,6-diol (H₂O)₂ (54.8 g, 96%) as a white solid. ESI-MS m/z calc. 155.0695, found 156.2 (M+1)⁺; Retention time: 0.369 minutes. ¹H NMR (300 MHz, DMSO-d6) δ 0.87 (t, J=7.2 Hz, 3H), 2.03-2.23 (m, 2H), 6.28 (s, 2H), 10.02-10.45 (m, 2H).

### Step 2: N′-(4,6-Dichloro-5-ethyl-pyrimidin-2-yl)-N,N-dimethyl-formamidine

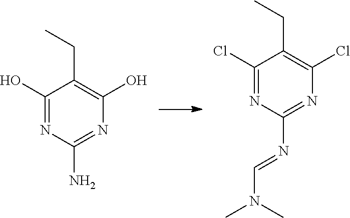

Oxalyl chloride (290.0 g, 200 mL, 2.285 mol) was slowly added to a solution of dimethylformamide (166.4 g, 177 mL, 2.277 mol) in chloroform (1.6 L) and the solution was stirred at room temperature for 30 minutes. 2-Amino-5-ethyl-pyrimidine-4,6-diol (H₂O)₂ (43.55 g, 227.8 mmol) was added then the reaction mixture was heated at 60° C. overnight. Once cooled to room temperature, the reaction mixture was diluted with saturated sodium bicarbonate solution (2.0 L) and stirred vigorously for 15 minutes. A 25% sodium hydroxide solution (250 mL) was added to reach pH ˜8-9. Layers were separated and aqueous layer was extracted with dichloromethane (2×700 mL). The organic layers were combined, dried over sodium sulfate and concentrated under reduced pressure to afford N′-(4,6-dichloro-5-ethyl-pyrimidin-2-yl)-N,N-dimethyl-formamidine (192 g, 341%) as brown oil. The crude material was used for the next step without any further purification. ESI-MS m/z calc. 246.0439, found 247.1 (M+1)⁺; Retention time: 1.25 minutes.

### Step 3: 4,6-Dichloro-5-ethyl-pyrimidin-2-amine

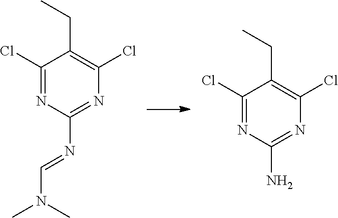

Concentrated hydrochloric acid (117 mL of 12 M, 1.404 mol) was added to a solution of N′-(4,6-dichloro-5-ethyl-pyrimidin-2-yl)-N,N-dimethyl-formamidine (57.9 g, 234.3 mmol) and the mixture was stirred at 50° C. for 150 minutes. The mixture was cooled in the freezer overnight then the solid was filtered, rinsed with cold isopropanol (350 mL) and dried to afford 4,6-dichloro-5-ethyl-pyrimidin-2-amine (35.4 g, 77%) as an off-white solid. ESI-MS m/z calc. 191.0017, found 192.1 (M+1)⁺; Retention time: 2.39 minutes. ¹H NMR (300 MHz, CDCl₃) ppm 1.15 (t, J=7.5 Hz, 3H), 2.75 (q, J=7.3 Hz, 2H), 5.30 (br. s., 2H).

### Step 4: tert-butyl N-tert-butoxycarbonyl-N-(4,6-dichloro-5-ethyl-pyrimidin-2-yl)carbamate

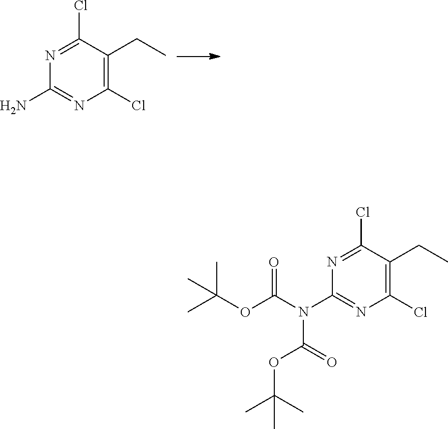 [SMILES: CCc1c(Cl)nc(N)nc1Cl ; CCc1c(Cl)nc(N(C(=O)OC(C)(C)C)C(=O)OC(C)(C)C)nc1Cl]

4,6-dichloro-5-ethyl-pyrimidin-2-amine (973 mg, 5.066 mmol) and Boc anhydride (2.36 g, 10.81 mmol) were dissolved in DCM (7.5 mL), followed by DMAP (50.5 mg, 0.4134 mmol). The reaction mixture was stirred at room temperature for 16 h. The reaction mixture was diluted with DCM (25 mL) and washed with water, then brine. The organics were dried over sodium sulfate and evaporated to give tert-butyl N-tert-butoxycarbonyl-N-(4,6-dichloro-5-ethyl-pyrimidin-2-yl)carbamate (1.94 g, 98%). ESI-MS m/z calc. 391.10657, found 392.1 (M+1)⁺; Retention time: 0.84 minutes; LC method B. ¹H NMR (400 MHz, Chloroform-d) δ 2.92 (q, J=7.5 Hz, 2H), 1.48 (s, 18H), 1.23 (t, J=7.5 Hz, 3H).

### Step 5: tert-Butyl N-tert-butoxycarbonyl-N-[4-chloro-6-(2,6-dimethylphenyl)-5-ethyl-pyrimidin-2-yl]carbamate

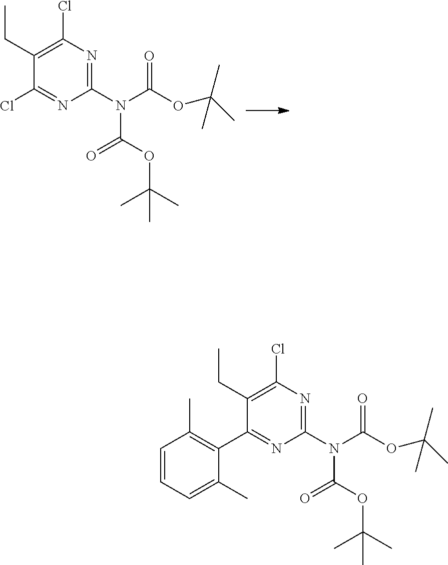 [SMILES: CCc1c(Cl)nc(N(C(=O)OC(C)(C)C)C(=O)OC(C)(C)C)nc1Cl ; CCc1c(Cl)nc(N(C(=O)OC(C)(C)C)C(=O)OC(C)(C)C)nc1-c1c(C)cccc1C]

To a solution of tert-butyl N-tert-butoxycarbonyl-N-(4,6-dichloro-5-ethyl-pyrimidin-2-yl)carbamate (34.92 g, 89.019 mmol) dissolved in DME (250 mL) and water (50 mL) was added (2,6-dimethylphenyl)boronic acid (13.35 g, 89.010 mmol) and cesium carbonate (75.4 g, 231.42 mmol) at room temperature. The solution was stirred for 10 min while being bubbled with a nitrogen stream. Then Pd(dppf)Cl₂ (5.2 g, 7.1067 mmol) was added to the solution and heated to 80° C. overnight. The solution was cooled to room temperature before being diluted with water (250 mL) and extracted with ethyl acetate (2×300 mL). The combined organic layer was washed with brine (400 mL) and dried over sodium sulfate before being concentrated in vacuum to give tert-butyl N-tert-butoxycarbonyl-N-[4-chloro-6-(2,6-dimethylphenyl)-5-ethyl-pyrimidin-2-yl]carbamate (37.51 g, 43%). ESI-MS m/z calc. 461.20813, found 462.2 (M+1)⁺; Retention time: 3.85 minutes; LC method E.

### Step 6: 4-Chloro-6-(2,6-dimethylphenyl)-5-ethyl-pyrimidin-2-amine

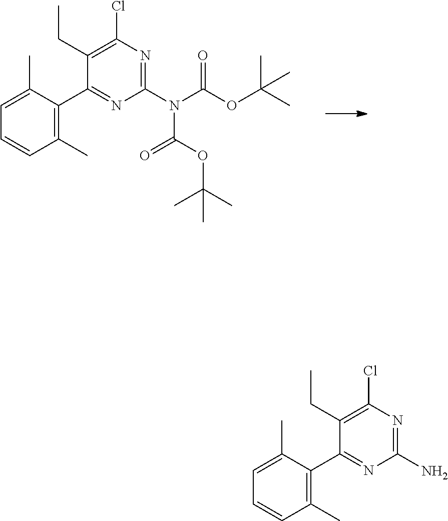 [SMILES: CCc1c(Cl)nc(N(C(=O)OC(C)(C)C)C(=O)OC(C)(C)C)nc1-c1c(C)cccc1C ; CCc1c(Cl)nc(N)nc1-c1c(C)cccc1C]

tert-Butyl N-tert-butoxycarbonyl-N-[4-chloro-6-(2,6-dimethylphenyl)-5-ethyl-pyrimidin-2-yl]carbamate (393 mg, 0.8507 mmol) and HCl in dioxane (3 mL of 4 M, 12.00 mmol) were combined in dichloromethane (4 mL) and stirred for 16 h. The reaction mixture was evaporated to dryness. The resulting material was partitioned between ethyl acetate and a saturated sodium bicarbonate solution and the mixture was stirred for 15 min. The organics were separated, washed with brine, dried over sodium sulfate and evaporated to give 4-chloro-6-(2,6-dimethylphenyl)-5-ethyl-pyrimidin-2-amine (219 mg, 98%). ESI-MS m/z calc. 261.10327, found 262.1 (M+1)⁺; Retention time: 0.64 minutes; LC method B.

### Step 7: Methyl 3-[[4-chloro-6-(2,6-dimethylphenyl)-5-ethyl-pyrimidin-2-yl]sulfamoyl]benzoate

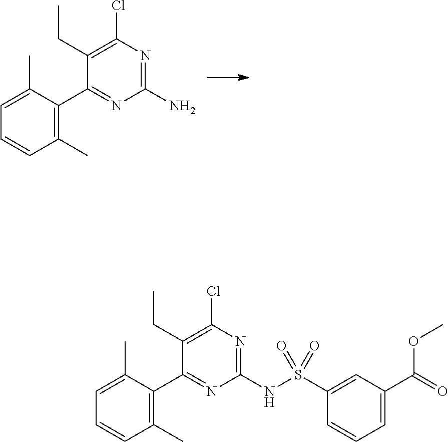 [SMILES: CCc1c(Cl)nc(N)nc1-c1c(C)cccc1C ; CCc1c(Cl)nc(NS(=O)(=O)c2cccc(C(=O)OC)c2)nc1-c1c(C)cccc1C]

4-Chloro-6-(2,6-dimethylphenyl)-5-ethyl-pyrimidin-2-amine (219 mg, 0.8367 mmol) was dissolved in THE (2.2 mL) and cooled in an ice bath. methyl 3-chlorosulfonylbenzoate (607.2 mg, 2.588 mmol) was added in one portion. lithium tert-amoxide (1.5 mL of 40% w/w, 4.656 mmol) was added dropwise and the reaction was allowed to slowly warm to room temperature. The reaction was stirred for 5 h, then made acidic by the addition of 1 M HCl. The reaction mixture was extracted with ethyl acetate. The organics were washed with brine, dried over sodium sulfate and evaporated. The crude material was purified by silica gel chromatography eluting with 0-50% ethyl acetate in hexanes to give methyl 3-[[4-chloro-6-(2,6-dimethylphenyl)-5-ethyl-pyrimidin-2-yl]sulfamoyl]benzoate (294 mg, 76%). ESI-MS m/z calc. 459.10196, found 460.1 (M+1)⁺; Retention time: 0.77 minutes; LC method B.

### Step 8: 3-[[4-Chloro-6-(2,6-dimethylphenyl)-5-ethyl-pyrimidin-2-yl]sulfamoyl]benzoic acid

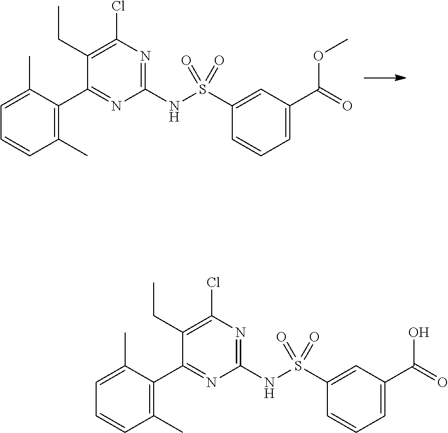 [SMILES: CCc1c(Cl)nc(NS(=O)(=O)c2cccc(C(=O)OC)c2)nc1-c1c(C)cccc1C ; CCc1c(Cl)nc(NS(=O)(=O)c2cccc(C(=O)O)c2)nc1-c1c(C)cccc1C]

To a solution of methyl 3-[[4-chloro-6-(2,6-dimethylphenyl)-5-ethyl-pyrimidin-2-yl]sulfamoyl]benzoate (4.75 g, 10.327 mmol) in THE (100 mL) was added an aqueous solution of NaOH (45 mL of 1 M, 45.000 mmol) and stirred for 1 hour at room temperature. The solution was acidified using 1 M HCl (75 mL) and extracted with ethyl acetate (2×100 mL) before being washed with brine (100 mL). The organic layer was dried over sodium sulfate and concentrated in vacuum to give 3-[[4-chloro-6-(2,6-dimethylphenyl)-5-ethyl-pyrimidin-2-yl]sulfamoyl]benzoic acid (3.83 g, 78%) as a white solid. ESI-MS m/z calc. 445.0863, found 446.2 (M+1)⁺; Retention time: 2.52 minutes; LC method E. ¹H NMR (500 MHz, DMSO-d6) δ 13.39 (s, 1H), 12.28 (s, 1H), 8.41 (s, 1H), 8.17 (dt, J=7.8, 1.4 Hz,1H), 8.09 (dt, J=7.9, 1.5 Hz, 1H), 7.66 (t, J=7.8 Hz, 1H), 7.26 (t, J=7.6 Hz, 1H), 7.13 (d, J=7.6 Hz, 2H), 2.26 (q, J=7.5 Hz, 2H), 1.75 (s, 6H), 0.83 (t, J=7.5 Hz, 3H).

### Example 102: Preparation of 3-[[4-[(2R)-2-Amino-4,4-dimethyl-pentoxy]-6-[2-(isopropoxymethyl)-6-methyl-phenyl]-5-methyl-pyrimidin-2-yl]sulfamoyl]benzoic acid Step 1: 2-Bromo-1-(isopropoxymethyl)-3-methyl-benzene

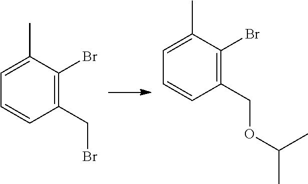

A slurry of NaH (13.1 g, 327.53 mmol) in THE (600 mL) at 0° C. was added isopropanol (23.550 g, 30 mL, 391.88 mmol) slowly, and then stirred for 30 min. A solution of 2-bromo-1-(bromomethyl)-3-methyl-benzene (45.5 g, 172.38 mmol) in THE (200 mL) was added to the reaction mixture. It was then warmed to room temperature, and then stirred overnight. The reaction was quenched with saturated NH₄Cl (400 mL) and extracted with Et₂O (3×250 mL). The combined organic extracts were dried over anhydrous sodium sulfate and concentrated under vacuum. The residue was purified by silica gel chromatography using 0 to 20% DCM in hexane to furnish 2-bromo-1-(isopropoxymethyl)-3-methyl-benzene (41.5 g, 96%) as a light yellow oil. ¹H NMR (500 MHz, Chloroform-d) δ 7.34 (d, J=8.1 Hz, 1H), 7.20 (t, J=7.5 Hz, 1H), 7.15 (d, J=7.3 Hz, 1H), 4.57 (s, 2H), 3.77-3.72 (m, 1H), 2.42 (s, 3H), 1.26 (d, J=6.1 Hz, 6H). ESI-MS m/z calc. 242.03062, Retention time: 3.84 minutes; LC Method E.

### Step 2: 2-[2-(Isopropoxymethyl)-6-methyl-phenyl]-4,4,5,5-tetramethyl-1,3,2-dioxaborolane

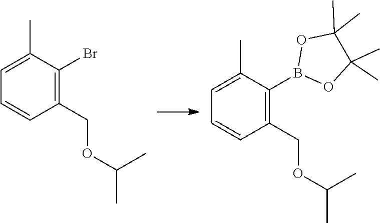 [SMILES: Cc1cccc(COC(C)C)c1Br ; Cc1cccc(COC(C)C)c1B1OC(C)(C)C(C)(C)O1]

A solution of 2-bromo-1-(isopropoxymethyl)-3-methyl-benzene (41.5 g, 136.55 mmol) in anhydrous dioxane (450 mL) was added KOAc (33.5 g, 341.34 mmol). The mixture was degassed with nitrogen for 5 min before adding 4,4,5,5-tetramethyl-2-(4,4,5,5-tetramethyl-1,3,2-dioxaborolan-2-yl)-1,3,2-dioxaborolane (62.4 g, 245.73 mmol) and Pd(dppf)Cl₂ (9.8 g, 13.393 mmol), and then degassed for another 5 min. The tube was then sealed and heated to 100° C. and stirred for 18 h. After the reaction was cooled to room temperature, saturated ammonium chloride (200 mL) was added and extracted with ethyl acetate (3×200 mL). The combined organic extracts washed with brine (250 mL), dried over sodium sulfate, filtered, and concentrated. The residue was purified by silica gel chromatography using 0 to 20% EtOAc in hexane to furnish 2-[2-(isopropoxymethyl)-6-methyl-phenyl]-4,4,5,5-tetramethyl-1,3,2-dioxaborolane (30.37 g, 77%) as a light green oil. ESI-MS m/z calc. 290.20532, found 291.3 (M+1)⁺; Retention time: 3.9 minutes; LC Method E.

### Step 3: tert-Butyl N-tert-butoxycarbonyl-N-[4-chloro-6-[2-(isopropoxymethyl)-6-methyl-phenyl]-5-methyl-pyrimidin-2-yl]carbamate

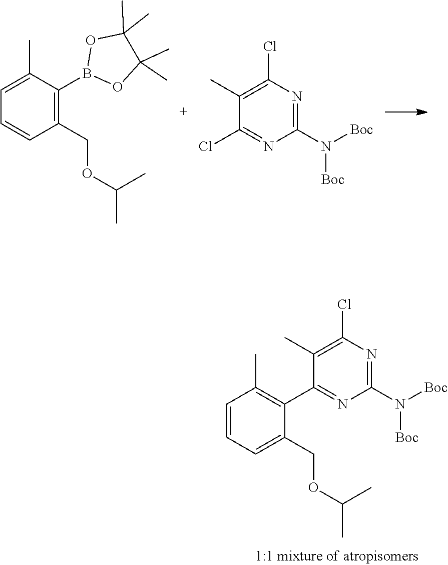

A reaction flask was charged with 2-[2-(isopropoxymethyl)-6-methyl-phenyl]-4,4,5,5-tetramethyl-1,3,2-dioxaborolane (30.37 g, 104.65 mmol), tert-butyl N-tert-butoxycarbonyl-N-(4,6-dichloro-5-methyl-pyrimidin-2-yl)carbamate (44.6 g, 112.02 mmol) and Cs₂CO₃ (85.2 g, 261.50 mmol) in a solvent mixture of DME (585 mL) and water (115 mL). The reaction was purged with argon for 5 min. Pd(dppf)Cl₂ (6.2 g, 8.4734 mmol) was added to reaction mixture. The reaction mixture was purged with argon for another 5 min. The reaction mixture was stirred at 85° C. for 3.5 h. The reaction was cooled to rt and diluted with water (500 mL). The aqueous layer was separated and extracted with EtOAc (3×400 mL). The combined organic layers were washed with brine (500 mL), dried over anhydrous sodium sulfate and concentrated under vacuum. The residue was purified by silica gel chromatography using 0 to 25% ethyl acetate in hexane to furnish tert-butyl N-tert-butoxycarbonyl-N-[4-chloro-6-[2-(isopropoxymethyl)-6-methyl-phenyl]-5-methyl-pyrimidin-2-yl]carbamate (32.8 g, 62%) as a yellow gel. ESI-MS m/z calc. 505.23434, found 506.3 (M+1)⁺; Retention time: 4.29 minutes; LC Method E.

### Step 4: 4-Chloro-6-[2-(isopropoxymethyl)-6-methyl-phenyl]-5-methyl-pyrimidin-2-amine

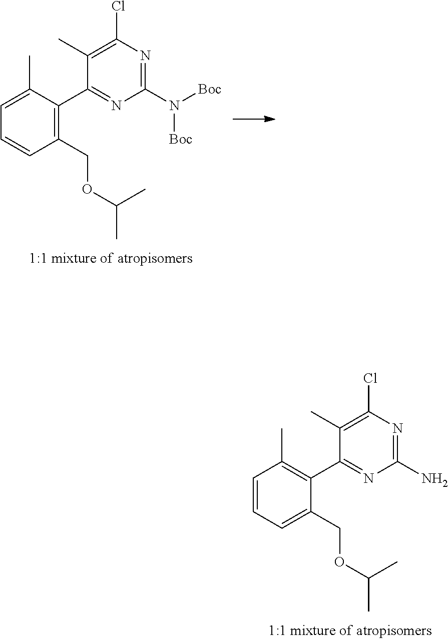 [SMILES: Cc1cccc(COC(C)C)c1-c1nc(N(C(=O)OC(C)(C)C)C(=O)OC(C)(C)C)nc(Cl)c1C ; Cc1cccc(COC(C)C)c1-c1nc(N)nc(Cl)c1C]

To a solution of tert-butyl N-tert-butoxycarbonyl-N-[4-chloro-6-[2-(isopropoxymethyl)-6-methyl-phenyl]-5-methyl-pyrimidin-2-yl]carbamate (32.89 g, 63.046 mmol) in DCM (300 mL) was added a solution of HCl in dioxane (140 mL of 4 M, 560.0 mmol) at 0° C. The reaction was stirred at room temperature overnight. The volatiles were removed under vacuum, and the resulting solid was triturated with diethyl ether (250 mL). The white solid was dissolved with DCM (500 mL), washed with saturated sodium bicarbonate (300 mL), dried over anhydrous sodium sulfate filtered and dried in vacuo to furnish 4-chloro-6-[2-(isopropoxymethyl)-6-methyl-phenyl]-5-methyl-pyrimidin-2-amine (18.929 g, 97%). ¹H NMR (500 MHz, DMSO-d6) δ 7.32-7.21 (m, 3H), 6.87 (s, 2H), 4.13 (d, J=2.6 Hz, 2H), 3.38-3.33 (m, 1H), 1.98 (s, 3H), 1.78 (s, 3H), 0.99 (d, J=6.1 Hz, 3H), 0.89 (d, J=6.1 Hz, 3H). ESI-MS m/z calc. 305.1295, found 306.4 (M+1)⁺; Retention time: 2.39 minutes; LC Method H.

### Step 5: Methyl 3-[[4-chloro-6-[2-(isopropoxymethyl)-6-methyl-phenyl]-5-methyl-pyrimidin-2-yl]sulfamoyl]benzoate

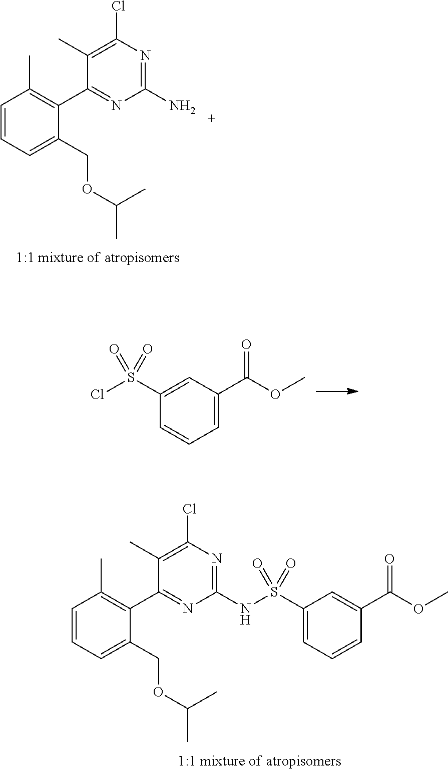 [SMILES: Cc1cccc(COC(C)C)c1-c1nc(N)nc(Cl)c1C ; COC(=O)c1cccc(S(=O)(=O)Cl)c1 ; COC(=O)c1cccc(S(=O)(=O)Nc2nc(Cl)c(C)c(-c3c(C)cccc3COC(C)C)n2)c1]

To a solution of 4-chloro-6-[2-(isopropoxymethyl)-6-methyl-phenyl]-5-methyl-pyrimidin-2-amine (1 g, 3.270 mmol) and methyl 3-chlorosulfonylbenzoate (2.33 g, 9.929 mmol) in THE (20 mL) was added a solution of LiHMDS in THE (13.2 mL of 1.0 M, 13.20 mmol) dropwise over 10 min at −78° C. in a dry ice/acetone bath. The reaction mixture was stirred for 3 h at −78° C., and then quenched with 1 N HCl solution. The reaction was warmed to room temperature, diluted with ethyl acetate, and the layers were separated. The aqueous layer was extracted with ethyl acetate (3×). The combined organic layer was washed with brine, dried over sodium sulfate, filtered and concentrated in vacuo. The resulting yellow oil was purified by silica gel chromatography, eluting with a 0-100% gradient of ethyl acetate in hexanes, to give methyl 3-[[4-chloro-6-[2-(isopropoxymethyl)-6-methyl-phenyl]-5-methyl-pyrimidin-2-yl]sulfamoyl]benzoate (1.09 g, 66%) ESI-MS m/z calc. 503.12817, found 504.3 (M+1)⁺; Retention time: 0.74 minutes; LC Method B.

### Step 6: 3-[[4-Chloro-6-[2-(isopropoxymethyl)-6-methyl-phenyl]-5-methyl-pyrimidin-2-yl]sulfamoyl]benzoic acid

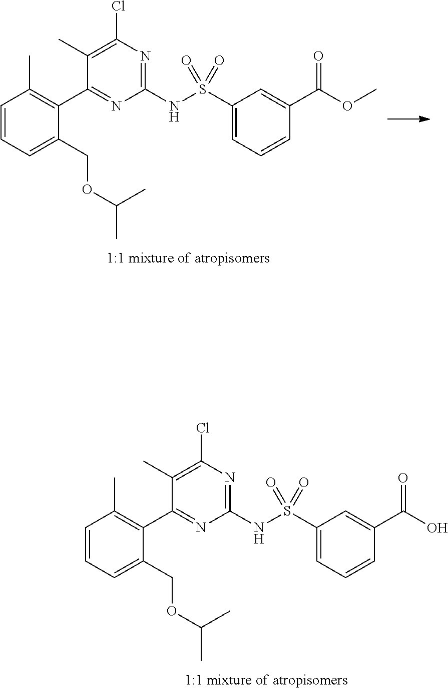 [SMILES: COC(=O)c1cccc(S(=O)(=O)Nc2nc(Cl)c(C)c(-c3c(C)cccc3COC(C)C)n2)c1 ; Cc1cccc(COC(C)C)c1-c1nc(NS(=O)(=O)c2cccc(C(=O)O)c2)nc(Cl)c1C]

Methyl 3-[[4-chloro-6-[2-(isopropoxymethyl)-6-methyl-phenyl]-5-methyl-pyrimidin-2-yl]sulfamoyl]benzoate (1.09 g, 2.163 mmol) was dissolved in THE (15 mL). The reaction was cooled in an ice bath, then aqueous NaOH (12 mL of 1 M, 12.0 mmol) was added. After 5 min the ice bath was removed, and the reaction mixture was stirred vigorously for 3 h. The reaction mixture was then partitioned between 1 N HCl solution and ethyl acetate. The layers were separated, and the aqueous layer was extracted with ethyl acetate (3×). The combined organics were washed with brine, dried over sodium sulfate, and concentrated to give a white solid, 3-[[4-chloro-6-[2-(isopropoxymethyl)-6-methyl-phenyl]-5-methyl-pyrimidin-2-yl]sulfamoyl]benzoic acid (1.007 g, 95%) ESI-MS m/z calc. 489.11252, found 490.3 (M+1)⁺; Retention time: 0.65 minutes; LC Method B.

### Step 7: 3-[[4-[(2R)-2-Amino-4,4-dimethyl-pentoxy]-6-[2-(isopropoxymethyl)-6-methyl-phenyl]-5-methyl-pyrimidin-2-yl]sulfamoyl]benzoic acid

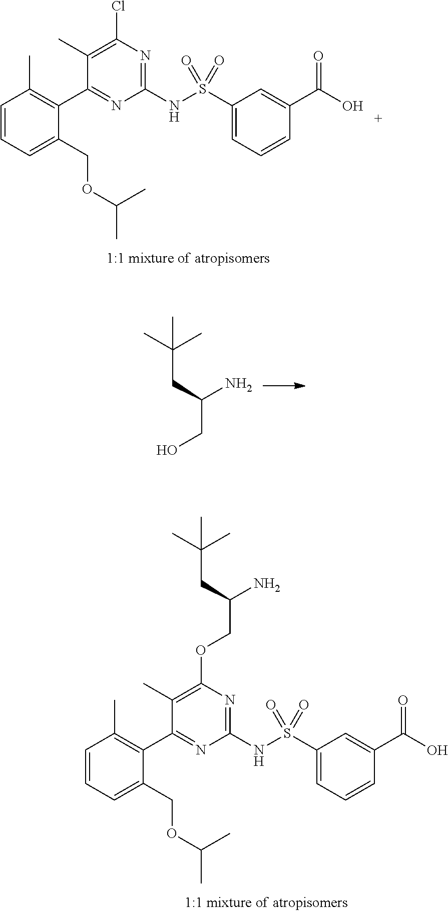

3-[[4-Chloro-6-[2-(isopropoxymethyl)-6-methyl-phenyl]-5-methyl-pyrimidin-2-yl]sulfamoyl]benzoic acid (700 mg, 1.429 mmol) was stirred with (2R)-2-amino-4,4-dimethyl-pentan-1-ol (hydrochloride salt) (255 mg, 1.521 mmol) in anhydrous THE (3.5 mL) at room temperature for 5 min. Then, sodium tert-butoxide (825 mg, 8.585 mmol) was added to the reaction mixture. A mild exotherm was observed and stirring was continued with no external heating for an additional 15 min. The reaction mixture was then partitioned between aqueous 1 N HCl and ethyl acetate. The layers were separated, and the aqueous layer was extracted with ethyl acetate (3×). The combined organics were washed with brine, dried over sodium sulfate and concentrated. The resulting solid was triturated with hexanes/ethyl acetate, then collected by filtration and dried to give a white solid, 3-[[4-[(2R)-2-amino-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (485.7 mg, 55%) ESI-MS m/z calc. 584.26685, found 585.4 (M+1)⁺; Retention time: 0.49 minutes; LC Method B.

### Example 103: Preparation of Compound IV-1

### Step 1. 6-Chloro-3-(3,3-dimethylbut-1-ynyl)pyrazin-2-amine

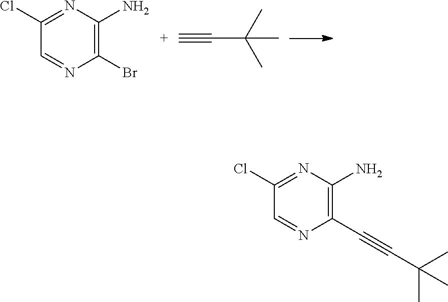

In a stirred solution of 3-bromo-6-chloro-pyrazin-2-amine (10 g, 47.975 mmol) in triethylamine (100 mL) nitrogen was bubbled for 10 minutes. 3,3-Dimethylbut-1-yne (3.9200 g, 5.6 mL, 47.721 mmol) was added, followed by bis(triphenylphosphine)palladium(II) dichloride (1.67 g, 2.3793 mmol) and copper iodide (450 mg, 2.3628 mmol). Nitrogen was bubbled into the mixture for 5 minutes. The mixture was stirred at room temperature for 3 hours. The mixture was diluted by EtOAc (200 mL), was filtered on a pad of Celite and the pad was rinsed with EtOAc (250 mL). Water (250 mL) was then added and the filtrate was washed with water (3×250 mL) and brine (1×200 mL), dried over anhydrous sodium sulfate, filtered and concentrated in vacuo to afford crude of 6-chloro-3-(3,3-dimethylbut-1-ynyl)pyrazin-2-amine (10 g, 94%) as an brownish solid; ¹H NMR (400 MHz, DMSO-d6) δ 7.72 (s, 1H), 6.85 (br. s., 2H), 1.31 (s, 9H). ESI-MS m/z calc. 209.072, found 210.2 (M+1)⁺; Retention time: 1.76 minutes. The crude was used for the next step without further purification. ESI-MS m/z calc. 209.07198, found 210.2 (M+1)⁺; Retention time: 1.76 minutes; LC method I.

### Step 2: 6-tert-Butyl-3-chloro-5-methyl-pyrrolo[2,3-b]pyrazine

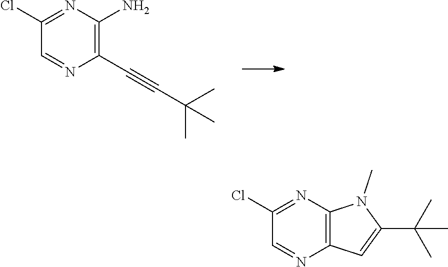

To a solution of 6-chloro-3-(3,3-dimethylbut-1-ynyl)pyrazin-2-amine (8.8 g, 41.466 mmol) in DMF (120 mL) was added potassium tert-butoxide (24 g, 213.88 mmol). The reaction mixture was heated at 80° C. for 30 minutes and was allowed to cool down to room temperature. To the reaction mixture at room temperature was added Mel (11.400 g, 5 mL, 80.316 mmol) dropwise over 20 minutes and the reaction was stirred for 45 minutes. To the reaction mixture at room temperature was again added Mel (11.400 g, 5 mL, 80.316 mmol) dropwise over 20 minutes and the reaction was stirred for 15 minutes. A saturated aqueous solution of sodium thiosulfate (50 mL) was added, and the mixture was stirred for 1 hour. The reaction mixture was diluted with water (100 mL), brine (100 mL) and ethyl acetate (100 mL) and the phases were separated. The aqueous layer was extracted with ethyl acetate (3×50 mL). The combined organic layers were washed with brine (3×150 mL), dried over anhydrous sodium sulfate, filtered and concentrated under reduced pressure to afford crude 6-tert-butyl-3-chloro-5-methyl-pyrrolo[2,3-b]pyrazine (9.6 g, 76%) as dark red oil. ¹H NMR (400 MHz, CDCl₃) δ 8.34 (s, 1H), 6.49 (s, 1H), 3.99 (s, 3H), 1.51 (s, 9H). ESI-MS m/z calc. 223.0876, found 224.2 (M+1)⁺; Retention time: 1.93 minutes. LC method I.

### Step 3: Methyl 6-tert-butyl-5-methyl-pyrrolo[2,3-b]pyrazine-3-carboxylate

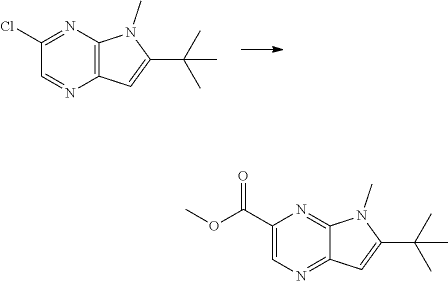 [SMILES: Cn1c(C(C)(C)C)cc2ncc(Cl)nc21 ; COC(=O)c1cnc2cc(C(C)(C)C)n(C)c2n1]

To a suspension of 6-tert-butyl-3-chloro-5-methyl-pyrrolo[2,3-b]pyrazine (8.65 g, 36.850 mmol) in MeOH (50 mL) was added triethylamine (6.5340 g, 9 mL, 64.572 mmol). Nitrogen was bubbled into the mixture for 15 minutes and then 1,1′-bis(diphenylphosphino)ferrocene palladium(II) chloride, complex with dichloromethane (906 mg, 1.1094 mmol) was added. Nitrogen bubbling was then continued for 5 minutes. The mixture was then stirred at 80° C. under 60 psi carbon monoxide pressure for 18 hours. The mixture was then allowed to cool down to room temperature, was filtered on a Celite pad followed by a silica gel pad and concentrated in vacuo. Water (100 mL) and EtOAc (100 mL) were then added, and the mixture was extracted with EtOAc (3×100 mL). The combined organic layers were washed with brine (100 mL) and dried over anhydrous sodium sulfate, filtered and concentrated in vacuo to afford crude methyl 6-tert-butyl-5-methyl-pyrrolo[2,3-b]pyrazine-3-carboxylate (9.53 g, 90%) as an orange oil. It was engaged in the following step without any further purification. ¹H NMR (400 MHz, CDCl₃) δ 9.17 (s, 1H), 6.60 (s, 1H), 4.10 (s, 3H), 4.04 (s, 3H), 1.55 (s, 9H). ESI-MS m/z calc. 247.1321, found 248.2 (M+1)⁺; Retention time: 1.7 minutes. LC method I.

### Step 4: (6-tert-Butyl-5-methyl-pyrrolo[2,3-b]pyrazin-3-yl)methanol

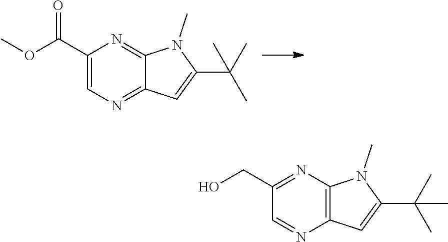 [SMILES: COC(=O)c1cnc2cc(C(C)(C)C)n(C)c2n1 ; Cn1c(C(C)(C)C)cc2ncc(CO)nc21]

To a stirred solution of methyl 6-tert-butyl-5-methyl-pyrrolo[2,3-b]pyrazine-3-carboxylate (5.3 g, 17.210 mmol) in methanol (50 mL) was added lithium borohydride (1 g, 45.906 mmol) in four portions every 10 minutes. At the end of addition, the reaction was stirred at room temperature for 1 hour and 30 minutes. Acetone (15 mL) was added, and the reaction was stirred for 30 minutes at room temperature. Then, water was added, and the stirring was continued for 5 minutes. The reaction mixture was concentrated under reduced pressure, then solubilized in MTBE (55 mL) and washed with an aqueous solution of NaOH (2 N, 30 mL), water (30 mL), brine (30 mL), dried over anhydrous magnesium sulfate, and filtered. The solution was evaporated to give (6-tert-butyl-5-methyl-pyrrolo[2,3-b]pyrazin-3-yl)methanol (3.4 g, 65%) as a yellow solid. ESI-MS m/z calc. 219.1372, found 220.2 (M+1)⁺; Retention time: 1.47 minutes. ¹H NMR (400 MHz, CDCl₃) δ 8.36 (s, 1H), 6.49 (s, 1H), 4.90 (s, 2H), 4.02 (s, 3H), 3.23-3.17 (overlapped with MTBE, br. s., 1H), 1.52 (s, 9H). LC method I.

### Step 5: 6-tert-Butyl-5-methyl-pyrrolo[2,3-b]pyrazine-3-carbaldehyde

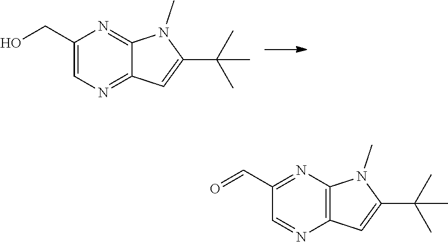 [SMILES: Cn1c(C(C)(C)C)cc2ncc(CO)nc21 ; Cn1c(C(C)(C)C)cc2ncc(C=O)nc21]

To a solution of (6-tert-butyl-5-methyl-pyrrolo[2,3-b]pyrazin-3-yl)methanol (3.3 g, 10.835 mmol) in DCM (100 mL) at 0° C. was added Dess-Martin periodinane (10 g, 23.577 mmol). The mixture was stirred for 30 minutes at 0° C. Then an aqueous solution of 10% sodium thiosulfate (30 mL) and a saturated aqueous solution of sodium bicarbonate (30 mL) were added, and the mixture was stirred for 1 hour. The layers were separated, and the organic phase was left standing overnight. A beige precipitate formed, and was filtered off, and the organic layer was dried over anhydrous sodium sulfate and evaporated in vacuo. The crude mixture was purified by silica gel chromatography (gradient: 0-40% EtOAc in heptanes, 18 CV), affording 6-tert-butyl-5-methyl-pyrrolo[2,3-b]pyrazine-3-carbaldehyde (2.4 g, 99%) as a light yellow solid. ¹H NMR (400 MHz, DMSO-d6) δ 10.10 (s, 1H), 8.93 (s, 1H), 6.69 (s, 1H), 4.06 (s, 3H), 1.51 (s, 9H). ESI-MS m/z calc. 217.1215, found 218.2 (M+1)⁺; Retention time: 3.81 minutes. LC method J.

### Step 6: 3-{[(1P,1P)-4-[(2R)-2-Amino-3-{bicyclo[1.1.1]pentan-1-yl}propoxy]-5-methyl-6-[2-methyl-6-(2-methylpropyl)phenyl]pyrimidin-2-yl]sulfamoyl}benzoic acid

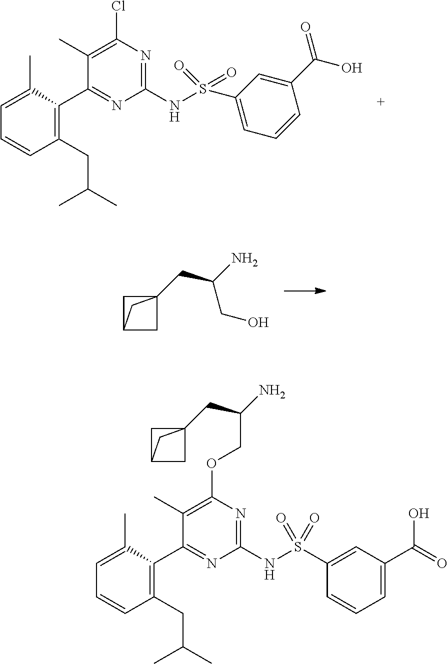

3-{[(1P)-4-Chloro-5-methyl-6-[2-methyl-6-(2-methylpropyl)phenyl]pyrimidin-2-yl]sulfamoyl}benzoic acid (500 mg, 1.055 mmol) and [(1R)-1-(1-bicyclo[1.1.1]pentanylmethyl)-2-hydroxy-ethyl]ammonium (hydrochloride salt) (226.8 mg, 1.277 mmol) were combined under nitrogen in anhydrous THE (5.3 mL). To the solution sodium tert-butoxide (406.8 mg, 4.233 mmol) was added in one portion resulting in a slightly exothermic reaction. The mixture was stirred at room temperature for 5.5 hours. The reaction was diluted with ethyl acetate (20 mL), 1 M HCl (20 mL), and brine (20 mL), and the resulting two phases were separated. The aqueous phase was further extracted with EtOAc (3×10 mL). The combined organic extracts were dried over magnesium sulfate, filtered, and concentrated under reduced pressure to give 3-{[(1P,1P)-4-[(2R)-2-amino-3-{bicyclo[1.1.1]pentan-1-yl}propoxy]-5-methyl-6-[2-methyl-6-(2-methylpropyl)phenyl]pyrimidin-2-yl]sulfamoyl}benzoic acid (hydrochloride salt) (574.1 mg, 57%) as a white solid. ESI-MS m/z calc. 578.2563, found 579.4 (M+1)⁺; Retention time: 1.29 minutes. LC method A.

### Step 7: 3-{[(]P)-4-[(2R)-3-{bicyclo[1.1.1]Pentan-1-yl}-2-[({6-tert-butyl-5-methyl-5H-pyrrolo[2,3-b]pyrazin-3-yl}methyl)amino]propoxy]-5-methyl-6-[2-methyl-6-(2-methylpropyl)phenyl]pyrimidin-2-yl]sulfamoyl}benzoic acid

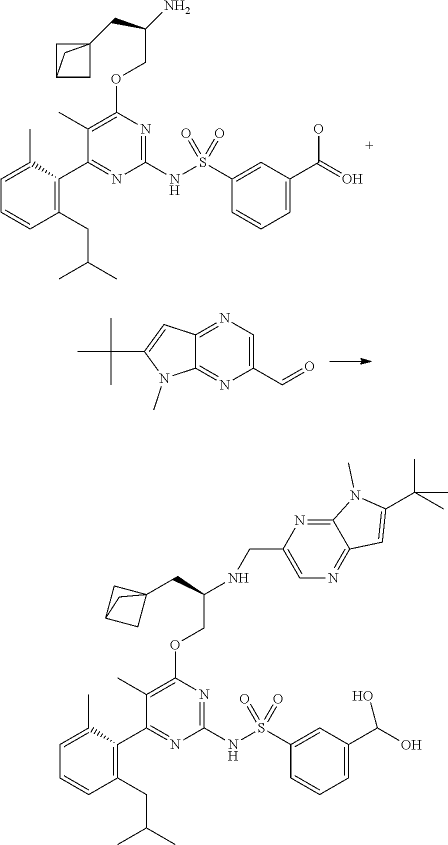

A 4 mL vial was charged under nitrogen with 3-{[(1P,1P)-4-[(2R)-2-amino-3-{bicyclo[1.1.1]pentan-1-yl}propoxy]-5-methyl-6-[2-methyl-6-(2-methylpropyl)phenyl]pyrimidin-2-yl]sulfamoyl}benzoic acid (hydrochloride salt) (72 mg, 0.09363 mmol), 6-tert-butyl-5-methyl-pyrrolo[2,3-b]pyrazine-3-carbaldehyde (21.6 mg, 0.09673 mmol), anhydrous DCM (450 μL), acetic acid (12 μL, 0.2110 mmol) and DIEA (57 μL, 0.3272 mmol). The mixture was cooled down in an ice bath, sodium triacetoxyborohydride (110 mg, 0.5190 mmol) was added and the reaction was stirred in the cooling bath for 2 h. The reaction was quenched with aqueous 1 M HCl (1 mL), MeOH (1 mL) and DMSO (1 mL), filtered and purified by reverse phase HPLC (1-99% acetonitrile/5 mM aqueous HCl over 15 min) to give 3-{[(1P)-4-[(2R)-3-{bicyclo[1.1.1]pentan-1-yl}-2-[({6-tert-butyl-5-methyl-5H-pyrrolo[2,3-b]pyrazin-3-yl}methyl)amino]propoxy]-5-methyl-6-[2-methyl-6-(2-methylpropyl)phenyl]pyrimidin-2-yl]sulfamoyl}benzoic acid (hydrochloride salt) (48.5 mg, 63%). ESI-MS m/z calc. 779.3829, found 780.86 (M+1)⁺; Retention time: 1.5 minutes. LC method A.

### Step 8: (5P,11R)-11-({bicyclo[1.1.1]Pentan-1-yl}methyl)-12-({6-tert-butyl-5-methyl-5H-pyrrolo[2,3-b]pyrazin-3-yl}methyl)-7-methyl-6-[2-methyl-6-(2-methylpropyl)phenyl]-9-oxa-2λ⁶-thia-3,5,12,19-tetraazatricyclo[12.3.1.1∧{4,8}]nonadeca-1(17),4(19),5,7,14(18),15-hexaene-2,2,13-trione (Compound IV-1

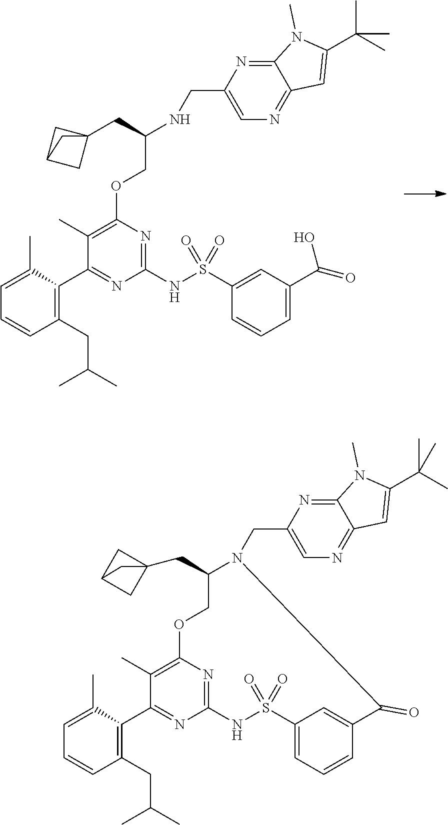

3-{[(1P)-4-[(2R)-3-{bicyclo[1.1.1]Pentan-1-yl}-2-[({6-tert-butyl-5-methyl-5H-pyrrolo[2,3-b]pyrazin-3-yl}methyl)amino]propoxy]-5-methyl-6-[2-methyl-6-(2-methylpropyl)phenyl]pyrimidin-2-yl]sulfamoyl}benzoic acid (hydrochloride salt) (46.1 mg, 0.05646 mmol) was combined under nitrogen with CDMT (34 mg, 0.1937 mmol) and DMF (2.5 mL). The solution was stirred in an ice bath. 4-Methyl-morpholine (39 μL, 0.3547 mmol) was added and the mixture was stirred in the ice bath that was allowed to warm to room temperature. After 20 h, the reaction was filtered and purified by reverse phase HPLC (10-99% acetonitrile/5 mM aqueous HCl over 15 min) to give (5P,11R)-11-({bicyclo[1.1.1]pentan-1-yl}methyl)-12-({6-tert-butyl-5-methyl-5H-pyrrolo[2,3-b]pyrazin-3-yl}methyl)-7-methyl-6-[2-methyl-6-(2-methylpropyl)phenyl]-9-oxa-2λ⁶-thia-3,5,12,19-tetraazatricyclo[12.3.1.1∧{4,8}]nonadeca-1(17),4(19),5,7,14(18),15-hexaene-2,2,13-trione (33.4 mg, 76%). ¹H NMR (400 MHz, Chloroform-d) δ 9.02 (t, J=1.8 Hz, 1H), 8.51 (s, 1H), 8.11 (d, J=7.9 Hz, 1H), 7.92 (d, J=7.6 Hz, 1H), 7.68 (t, J=7.8 Hz, 1H), 7.29-7.24 (overlapped with solvent, m, 1H), 7.13 (d, J=7.7 Hz, 1H), 7.08 (d, J=7.5 Hz, 1H), 6.69 (s, 1H), 5.63 (d, J=8.0 Hz, 1H), 5.44 (d, J=15.8 Hz, 1H), 4.38 (d, J=16.0 Hz, 1H), 4.19-4.03 (m, 5H), 2.45 (s, 1H), 2.28-2.10 (m, 2H), 2.05-1.94 (m, 1H), 1.91 (s, 3H), 1.84-1.77 (m, 2H), 1.71 (s, 3H), 1.59-1.54 (m, 6H), 1.52 (s, 9H), 0.86-0.74 (m, 6H). ESI-MS m/z calc. 761.3723, found 762.87 (M+1)⁺; Retention time: 2.11 minutes. LC method A.

### Example 104: Preparation of Compound IV-4

### Step 1: 3-[[4-[(2R)-2-Amino-3-isopropoxy-propoxy]-6-(2,6-dimethylphenyl)-5-ethyl-pyrimidin-2-yl]sulfamoyl]benzoic acid

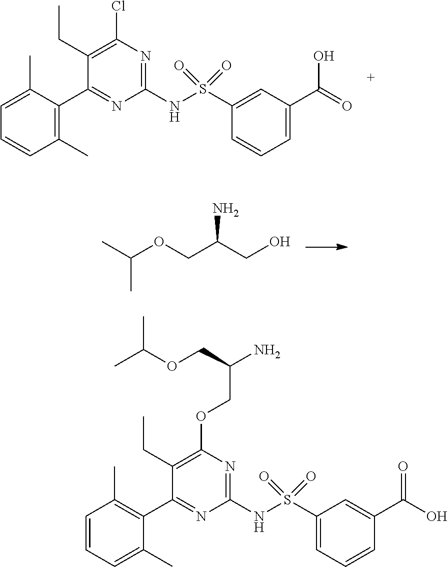

3-[[4-Chloro-6-(2,6-dimethylphenyl)-5-ethyl-pyrimidin-2-yl]sulfamoyl]benzoic acid (250 mg, 0.5606 mmol) and (2S)-2-amino-3-isopropoxy-propan-1-ol (hydrochloride salt) (116.3 mg, 0.6855 mmol) were combined under nitrogen in anhydrous THE (2.3 mL). To the solution sodium tert-butoxide (221.0 mg, 2.300 mmol) was added in one portion resulting in a slightly exothermic reaction. The mixture was stirred at room temperature for 30 minutes. The reaction was diluted with ethyl acetate (7 mL), 1 M HCl (7 mL), and brine (7 mL), and the resulting two phases were separated. The aqueous phase was further extracted with EtOAc (3×7 mL). The combined organic extracts were dried over magnesium sulfate, filtered, and concentrated under reduced pressure to give 3-[[4-[(2R)-2-amino-3-isopropoxy-propoxy]-6-(2,6-dimethylphenyl)-5-ethyl-pyrimidin-2-yl]sulfamoyl]benzoic acid (hydrochloride salt) (284.5 mg, 88%) as a tan solid. ESI-MS m/z calc. 542.2199, found 543.1 (M+1)⁺; Retention time: 1.21 minutes. LC method A.

### Step 2: 3-[[4-[(2R)-2-[(6-tert-Butyl-5-methyl-pyrrolo[2,3-b]pyrazin-3-yl)methylamino]-3-isopropoxy-propoxy]-6-(2,6-dimethylphenyl)-5-ethyl-pyrimidin-2-yl]sulfamoyl]benzoic acid

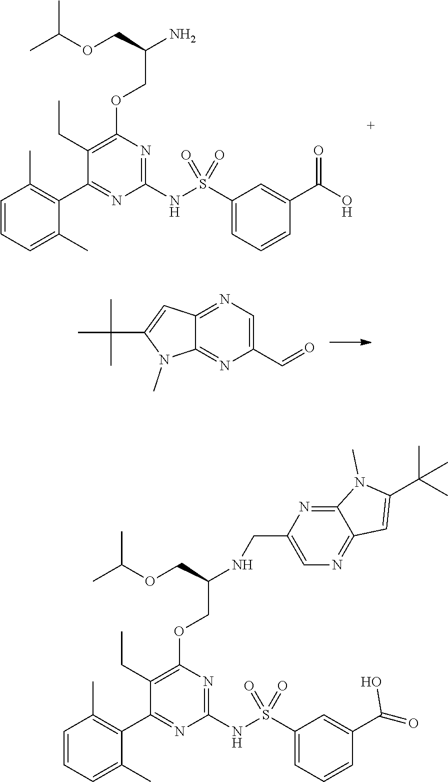 [SMILES: CCc1c(OC[C@H](N)COC(C)C)nc(NS(=O)(=O)c2cccc(C(=O)O)c2)nc1-c1c(C)cccc1C ; Cn1c(C(C)(C)C)cc2ncc(C=O)nc21 ; CCc1c(OC[C@@H](COC(C)C)NCc2cnc3cc(C(C)(C)C)n(C)c3n2)nc(NS(=O)(=O)c2cccc(C(=O)O)c2)nc1-c1c(C)cccc1C]

A 4 mL vial was charged under nitrogen with 3-[[4-[(2R)-2-amino-3-isopropoxy-propoxy]-6-(2,6-dimethylphenyl)-5-ethyl-pyrimidin-2-yl]sulfamoyl]benzoic acid (hydrochloride salt) (40 mg, 0.06907 mmol), 6-tert-butyl-5-methyl-pyrrolo[2,3-b]pyrazine-3-carbaldehyde (15.8 mg, 0.07272 mmol), anhydrous DCM (0.39 mL), and acetic acid (7.9 μL, 0.1389 mmol). The mixture was cooled down in an ice bath. DIPEA (36.1 μL, 0.2073 mmol) was added, followed by sodium triacetoxyborohydride (79.8 mg, 0.3765 mmol), and the reaction was vigorously stirred at 0° C. for 3 h. The reaction was quenched with 3 N aqueous HCl, diluted with MeOH (0.5 mL) and DMSO (0.5 mL), and the resulting solution was filtered. Purification by reverse phase HPLC (1-99% acetonitrile/5 mM aqueous HCl over 15 min) provided 3-[[4-[(2R)-2-[(6-tert-butyl-5-methyl-pyrrolo[2,3-b]pyrazin-3-yl)methylamino]-3-isopropoxy-propoxy]-6-(2,6-dimethylphenyl)-5-ethyl-pyrimidin-2-yl]sulfamoyl]benzoic acid (hydrochloride salt) (11.7 mg, 22%) as a white solid. ESI-MS m/z calc. 743.3465, found 744.3 (M+1)⁺; Retention time: 1.46 minutes. LC method A.

### Step 3: (11R)-12-[(6-tert-Butyl-5-methyl-pyrrolo[2,3-b]pyrazin-3-yl)methyl]-6-(2,6-dimethylphenyl)-7-ethyl-11-(isopropoxymethyl)-2,2-dioxo-9-oxa-2λ⁶-thia-3,5,12,19-tetrazatricyclo[12.3.1.14,8]nonadeca-1(17),4(19),5,7,14(18),15-hexaen-13-one (Compound IV-4

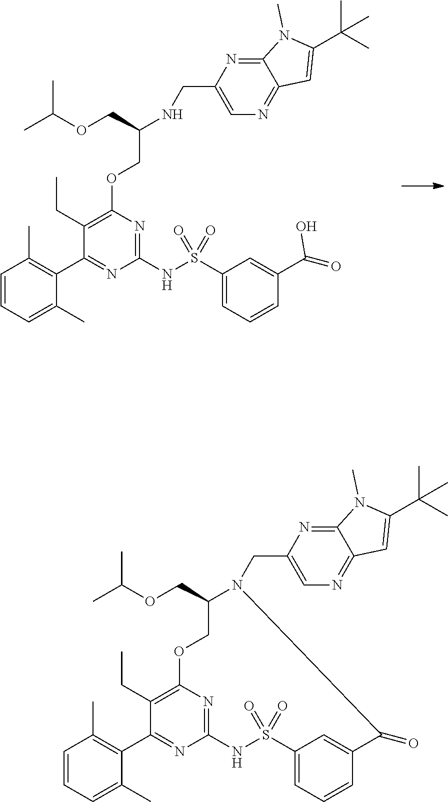

3-[[4-[(2R)-2-[(6-tert-Butyl-5-methyl-pyrrolo[2,3-b]pyrazin-3-yl)methylamino]-3-isopropoxy-propoxy]-6-(2,6-dimethylphenyl)-5-ethyl-pyrimidin-2-yl]sulfamoyl]benzoic acid (hydrochloride salt) (11.7 mg, 0.01499 mmol) was combined with CDMT (10.3 mg, 0.05867 mmol) in DMF (1.5 mL) and cooled to 0° C. N-methylmorpholine (10.0 μL, 0.09096 mmol) was added by syringe and the reaction was stirred at 0° C. for 30 minutes. The ice bath was then removed and stirring was continued for an additional 3 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO (0.5 mL) and MeOH (0.5 mL), filtered, and purified by reverse phase HPLC (1-99% acetonitrile/5 mM aqueous HCl over 25 min) to give (11R)-12-[(6-tert-butyl-5-methyl-pyrrolo[2,3-b]pyrazin-3-yl)methyl]-6-(2,6-dimethylphenyl)-7-ethyl-11-(isopropoxymethyl)-2,2-dioxo-9-oxa-2λ⁶-thia-3,5,12,19-tetrazatricyclo[12.3.1.14,8]nonadeca-1(17),4(19),5,7,14(18),15-hexaen-13-one (2.2 mg, 20%) as a yellow solid. ¹H NMR (400 MHz, CDCl₃) δ 8.94 (t, J=1.9 Hz, 1H), 8.49 (s, 1H), 8.04-7.99 (m, 1H), 7.89-7.83 (m, 1H), 7.64 (t, J=7.7 Hz, 1H), 7.25-7.21 (m, 1H), 7.11 (d, J=7.6 Hz, 1H), 7.08 (d, J=7.6 Hz, 1H), 6.73 (s, 1H), 5.55 (d, J=7.3 Hz, 1H), 5.49 (d, J=15.9 Hz, 1H), 4.85 (d, J=16.0 Hz, 1H), 4.49-4.37 (m, 2H), 4.16 (s, 3H), 3.73-3.62 (m, 2H), 3.54 (hept, J=6.0 Hz, 1H), 2.27-2.17 (m, 1H), 2.15-2.05 (m, 1H), 2.03 (s, 3H), 1.99 (s, 3H), 1.52 (s, 9H), 1.17 (d, J=6.0 Hz, 3H), 1.13 (d, J=6.1 Hz, 3H), 0.85 (t, J=7.4 Hz, 3H). ESI-MS m/z calc. 725.33594, found 726.3 (M+1)⁺; Retention time: 1.82 minutes. LC method A.

### Example 105: Preparation of Compound IV-13

### Step 1: (11R)-6-(2,6-Dimethylphenyl)-12-[[6-(1-hydroxy-3-methoxy-1,3-dimethyl-butyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-13

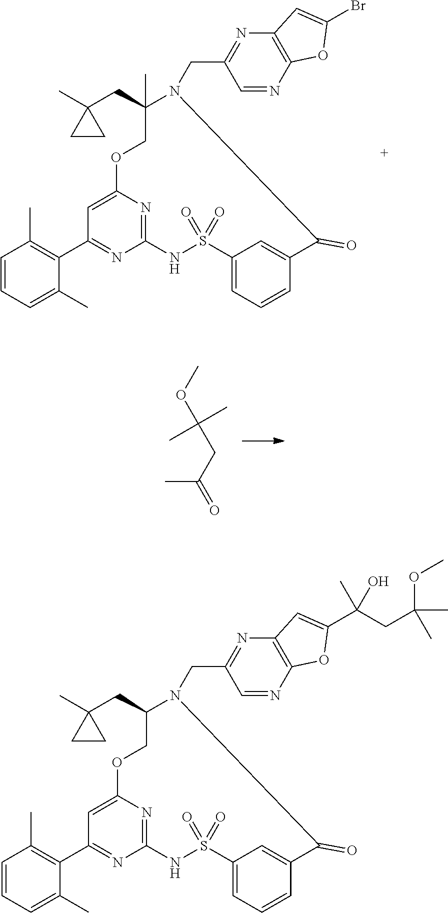

To a stirred solution of (11R)-12-[(6-bromofuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (25 mg, 0.03518 mmol) in anhydrous tetrahydrofuran (500 μL) was added sodium hydride (2 mg, 0.05000 mmol) (60% in mineral oil) at ambient temperature under nitrogen. After stirring for 15 min at ambient temperature, the heterogeneous mixture was cooled to −78° C. (dry ice-acetone bath) and n-butyllithium (2.5 M in hexanes) (25 μL of 2.5 M, 0.06250 mmol) was added dropwise over 1 min. After stirring the resulting yellow heterogeneous mixture at that temperature for 30 min, a solution of 4-methoxy-4-methyl-pentan-2-one (5.5 mg, 0.04225 mmol) in anhydrous tetrahydrofuran (100 μL) was added dropwise over 2 min. Then stirred for 15 min and the dry ice-acetone bath was removed and the reaction was allowed to warm to room temperature and stirred for 13 h. The reaction was quenched with glacial acetic acid (15 μL, 0.2638 mmol) and concentrated under reduced pressure. The residue was taken up in DMSO (3 mL) and the solution was micro-filtered and purified from preparative reverse phase HPLC (Cis) (5-99% acetonitrile in water over 20 min, HCl as a modifier). The desired fractions were combined and dried to furnish (11R)-6-(2,6-dimethylphenyl)-12-[[6-(1-hydroxy-3-methoxy-1,3-dimethyl-butyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (4 mg, 14%) as a white solid. ¹H NMR (400 MHz, MeOD) δ 8.76 (dt, J=9.0, 1.9 Hz, 1H), 8.37 (d, J=2.4 Hz, 1H), 8.03 (dd, J=7.8, 1.6 Hz, 1H), 7.74 (d, J=7.5 Hz, 1H), 7.67 (t, J=7.7 Hz, 1H), 7.26 (t, J=7.6 Hz, 1H), 7.14 (d, J=7.7 Hz, 2H), 7.01 (d, J=3.5 Hz, 1H), 6.27 (s, 1H), 5.60 (dd, J=10.8, 4.1 Hz, 1H), 5.12 (dd, J=15.5, 5.6 Hz, 1H), 4.52 (d, J=15.5 Hz, 1H), 4.48-4.38 (m, 1H), 4.38-4.28 (m, 1H), 3.14 (s, 3H), 2.32 (d, J=15.0 Hz, 1H), 2.18 (d, J=15.0 Hz, 1H), 2.10 (s, 6H), 1.84 (d, J=15.2 Hz, 1H), 1.59 (s, 3H), 1.62-1.55 (m, 1H), 1.17 (s, 3H), 1.01 (s, 3H), 0.50 (s, 3H), 0.43-0.34 (m, 1H), 0.25 (dt, J=9.6, 5.0 Hz, 1H), 0.15 (dt, J=9.3, 4.8 Hz, 1H), 0.09-0.02 (m, 1H). ESI-MS m/z calc. 754.3149, found 755.2 (M+1)⁺; Retention time: 1.85 minutes. LC method A.

### Example 106: Preparation of Compound IV-14 and Compound IV-15

### Step 1: 5-Chloro-1-[(4-methoxyphenyl)methyl]-3-[(E)-styryl]pyrazin-2-one

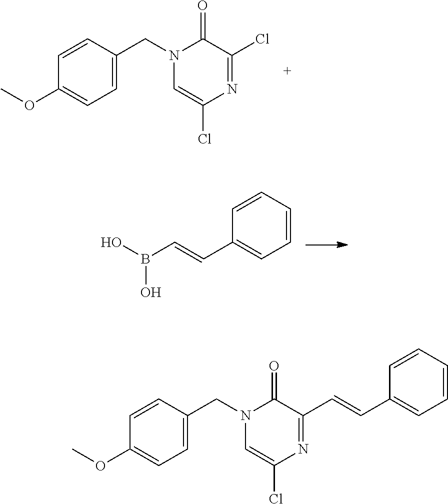 [SMILES: COc1ccc(Cn2cc(Cl)nc(Cl)c2=O)cc1 ; OB(O)/C=C/c1ccccc1 ; COc1ccc(Cn2cc(Cl)nc(/C=C/c3ccccc3)c2=O)cc1]

A mixture of 3,5-dichloro-1-[(4-methoxyphenyl)methyl]pyrazin-2-one (45 g, 156.25 mmol), [(E)-styryl]boronic acid (25.5 g, 172.34 mmol), and sodium carbonate (82.9 g, 782.16 mmol) in water (300 mL) and DME (1125 mL) at room temperature was degassed with nitrogen for 30 minutes. Pd(PPh₃)₄ (9.1 g, 7.8750 mmol) was added to the reaction mixture. The reaction mixture was refluxed in an oil bath for 3 h under argon atmosphere. The reaction was cooled to room temperature and diluted with DCM (500 mL), washed with water (500 mL), the aqueous layer extracted with DCM (2×500 mL). The combined organic layers were washed with brine (500 mL), dried over anhydrous sodium sulfate, filtered, and concentrated in vacuo. The residue was purified by silica gel chromatography using 0-40% EtOAc in hexanes to provide 5-chloro-1-[(4-methoxyphenyl)methyl]-3-[(E)-styryl]pyrazin-2-one (43.75 g, 79%) as yellowish brown solid. ESI-MS m/z calc. 352.0979, found 353.3 (M+1)⁺; Retention time: 3.33 minutes. ¹H NMR (500 MHz, DMSO-d6) δ 8.07 (s, 1H), 7.91 (d, J=16.2 Hz, 2H), 7.68 (d, J=7.1 Hz, 2H), 7.46-7.33 (m, 5H), 6.92 (d, J=8.8 Hz, 2H), 5.04 (s, 2H), 3.73 (s, 3H). LC method E.

### Step 2: Methyl 4-[(4-methoxyphenyl)methyl]-5-oxo-6-[(E)-styryl]pyrazine-2-carboxylate

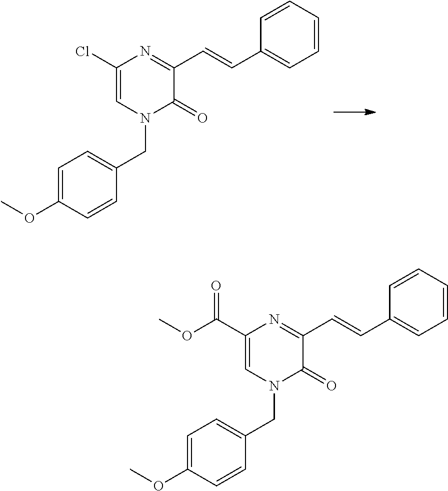 [SMILES: COc1ccc(Cn2cc(Cl)nc(/C=C/c3ccccc3)c2=O)cc1 ; COC(=O)c1cn(Cc2ccc(OC)cc2)c(=O)c(/C=C/c2ccccc2)n1]

To a stainless steel pressure vessel was added 5-chloro-1-[(4-methoxyphenyl)methyl]-3-[(E)-styryl]pyrazin-2-one (32.75 g, 92.825 mmol) Xantphos (4.4 g, 7.6043 mmol) and Pd(OAc)₂ (843 mg, 3.7549 mmol). Then degassed MeOH (300 mL), degassed DMF (50 mL) and degassed TEA (145.20 g, 200 mL, 1.4349 mol) were added. The vessel was sealed then pressurized with CO gas (120 psi) and vented 3 times. Finally, the vessel was pressurized with CO gas (120 psi) and heated at 100° C. for 24 h. The reaction was cooled to room temperature and Xantphos (8.8 g, 15.209 mmol), MeOH (100 mL) and Pd(OAc)₂ (1.688 g, 7.5187 mmol) were added and the vessel was sealed then pressurized with CO gas (120 psi) and vented 3 times. Finally, the vessel was pressurized with CO gas (120 psi) and heated at 85° C. for 48 h. The reaction was cooled to room temperature and Pd(dppf)Cl₂·DCM complex (1.75 g, 2.1429 mmol). The vessel was sealed then pressurized with CO gas (145 psi) and vented 3 times. Finally, the vessel was pressurized with CO gas (145 psi) and heated at 115° C. 24 h. The reaction was cooled to room temperature and concentrated in vacuo. The residue was diluted with EtOAc (1 L), washed with saturated aqueous ammonium chloride (500 mL) then washed with 1 M HCl (1 L). The aqueous layers were extracted with EtOAc (3×750 mL), washed with brine (2×750 mL), dried over magnesium sulfate, filtered and concentrated in vacuo. To the crude residue was added DMF (300 mL), potassium carbonate (38.5 g, 278.57 mmol) and Mel (41.040 g, 18 mL, 289.14 mmol). The reaction was stirred for 12 h at room temperature. The reaction was quenched with saturated aqueous ammonium chloride (750 mL), extracted with EtOAc (3×750 mL), washed with brine (3×500 mL) dried over magnesium sulfate, filtered, and concentrated in vacuo. The residue was purified in 2 batches by flash chromatography (each separately loaded onto a 330 g SiO₂ cartridge with toluene-DCM=10:1 and eluted with 0-27% EtOAc in hexanes over a 30 min gradient; held at 27% EtOAc for 60 min) and the product containing fractions were combined and concentrated to a low volume. The resulting crystalline solids were collected by vacuum filtration to provide methyl 4-[(4-methoxyphenyl)methyl]-5-oxo-6-[(E)-styryl]pyrazine-2-carboxylate (18 g, 52%) as yellow crystals. ESI-MS m/z calc. 376.1423, found 377.3 (M+1)⁺; Retention time: 5.17 minutes. ¹H NMR (500 MHz, Chloroform-d) δ 8.18 (d, J=16.2 Hz, 1H), 8.03 (s, 1H), 7.68-7.60 (m, 2H), 7.55 (d, J=16.2 Hz, 1H), 7.41-7.34 (m, 2H), 7.36-7.29 (m, 3H), 6.95-6.88 (m, 2H), 5.10 (s, 2H), 3.93 (s, 3H), 3.81 (s, 3H). LC method D.

### Step 3: Methyl 6-formyl-4-[(4-methoxyphenyl)methyl]-5-oxo-pyrazine-2-carboxylate

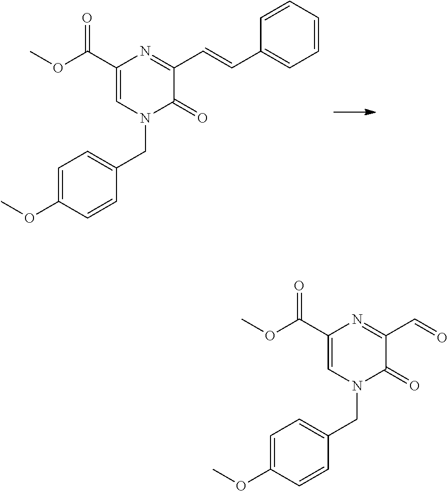 [SMILES: COC(=O)c1cn(Cc2ccc(OC)cc2)c(=O)c(/C=C/c2ccccc2)n1 ; COC(=O)c1cn(Cc2ccc(OC)cc2)c(=O)c(C=O)n1]

To a solution of methyl 4-[(4-methoxyphenyl)methyl]-5-oxo-6-[(E)-styryl]pyrazine-2-carboxylate (5.43 g, 14.426 mmol) in dioxane (54 mL) and t-BuOH (26 mL) was added Pyridine (3.2274 g, 3.3 mL, 40.802 mmol) followed by OsO₄ in water (15 mL of 4% w/v, 2.3601 mmol) then the reaction was stirred for 1 h at room temperature. Then NaIO₄ (12.5 g, 58.441 mmol) was added. The reaction was stirred for 1.5 h at room temperature then diluted with DCM (300 mL) and washed with saturated aqueous sodium bicarbonate (250 mL). The aqueous phase was extracted with DCM (2×300 mL), washed with brine, dried over magnesium sulfate, filtered, and concentrated in vacuo. The residue was purified by flash chromatography (loaded onto a 120 g SiO₂ cartridge with toluene-DCM=1:1, and eluted with 0-10% EtOAc in hexanes over a 10 min gradient, then 10-50% EtOAc in hexanes over a 15 min gradient) to provide methyl 6-formyl-4-[(4-methoxyphenyl)methyl]-5-oxo-pyrazine-2-carboxylate (2 g, 46%) as a yellow solid ESI-MS m/z calc. 302.0903, found 303.1 (M+1)⁺; Retention time: 2.54 minutes. ¹H NMR (500 MHz, Chloroform-d) δ 10.14 (s, 1H), 8.35 (s, 1H), 7.37-7.30 (m, 2H), 6.95-6.89 (m, 2H), 5.13 (s, 2H), 3.94 (s, 3H), 3.81 (s, 3H). LC method D.

### Step 4: Methyl 6-(2,2-dibromovinyl)-4-[(4-methoxyphenyl)methyl]-5-oxo-pyrazine-2-carboxylate

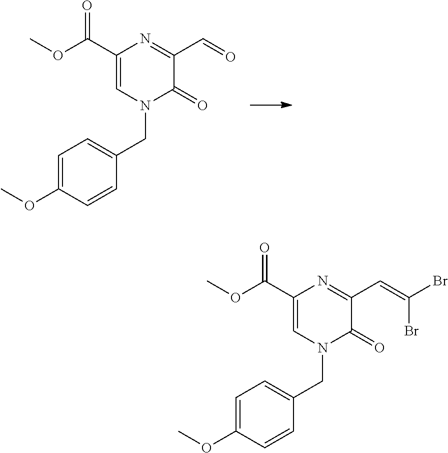 [SMILES: COC(=O)c1cn(Cc2ccc(OC)cc2)c(=O)c(C=O)n1 ; COC(=O)c1cn(Cc2ccc(OC)cc2)c(=O)c(C=C(Br)Br)n1]

To an oven dried flask, under nitrogen, was added CBr₄ (33.4 g, 100.72 mmol), triphenylphosphine (26.4 g, 100.65 mmol) and Zn (6.6 g, 100.93 mmol) followed by DCM (162 mL). The mixture was stirred for 14 h at room temperature. A solution of methyl 6-formyl-4-[(4-methoxyphenyl)methyl]-5-oxo-pyrazine-2-carboxylate (10.16 g, 33.611 mmol) in DCM (82 mL) was added to the PPh₃-Zn—CBr₄ mixture at room temperature and stirred 30 min then concentrated in vacuo to a low volume and filtered through celite. The pad of celite was washed with DCM (3×200 mL) and the combined filtrates were concentrated in vacuo. The residue was purified by flash chromatography (loaded onto a 330 g SiO₂ cartridge with DCM, and eluted with 0-5% EtOAc in hexanes over a 30 min gradient, then 5% EtOAc for 7 minutes, then 5-30% EtOAc over a 20 minute gradient) to provide methyl 6-(2,2-dibromovinyl)-4-[(4-methoxyphenyl)methyl]-5-oxo-pyrazine-2-carboxylate (10.97 g, 71%) as a white crystalline solid ESI-MS m/z calc. 455.932, found 456.8 (M+1)⁺; Retention time: 4.87 minutes. LC method D.

### Step 5: Methyl 6-bromofuro[2,3-b]pyrazine-2-carboxylate

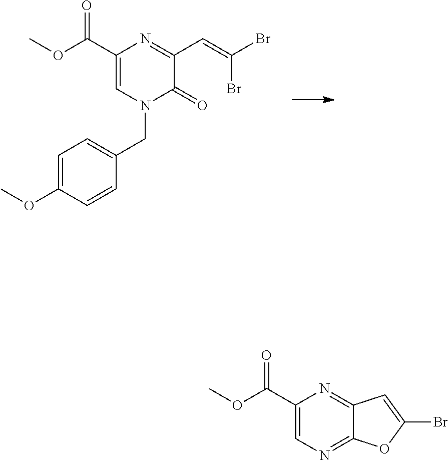 [SMILES: COC(=O)c1cn(Cc2ccc(OC)cc2)c(=O)c(C=C(Br)Br)n1 ; COC(=O)c1cnc2oc(Br)cc2n1]

To a pressure vessel containing methyl 6-(2,2-dibromovinyl)-4-[(4-methoxyphenyl)methyl]-5-oxo-pyrazine-2-carboxylate (4.6 g, 10.041 mmol) was added AgOTf (2.58 g, 10.041 mmol) and DCM (100 mL) under nitrogen and then the mixture was stirred for 15 minutes at room temperature. Then TFA (1.9357 g, 1.30 mL, 16.976 mmol) was added and the reaction was heated in a 100° C. oil bath for 3 h and 50 min. The mixture was cooled to room temperature, filtered through celite, and concentrated in vacuo. The residue was purified by flash chromatography (loaded onto a 120 g SiO₂ cartridge with DCM, and eluted with 0-20% EtOAc in hexanes over a 20 min gradient; then 20-30% EtOAc in hexanes over 7 minutes) to provide methyl 6-bromofuro[2,3-b]pyrazine-2-carboxylate (2.0 g, 77%) as a pale yellow solid ESI-MS m/z calc. 255.9484, found 257.3 (M+1)⁺; Retention time: 2.9 minutes. ¹H NMR (500 MHz, Chloroform-d) δ 9.06 (s, 1H), 7.15 (s, 1H), 4.07 (s, 3H). LC method D.

### Step 6: 6-Bromofuro[2,3-b]pyrazine-2-carbaldehyde

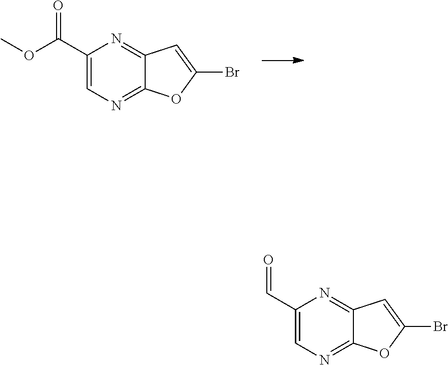

A solution of methyl 6-bromofuro[2,3-b]pyrazine-2-carboxylate (2 g, 7.7809 mmol) in DCM (30 mL) was cooled to −78° C. and the resulting suspension was slowly and dropwise added a solution of DIBAL in DCM (7.8 mL of 1 M, 7.8000 mmol). The yellow solution was stirred 30 minutes then DIBAL in DCM (2 mL of 1 M, 2.0000 mmol) was added. The reaction was stirred at this temperature and DIBAL in DCM (2 mL of 1 M, 2.0000 mmol) was added. The reaction was stirred for 30 minutes and methanol-water (1:1, 20 mL) was added. The mixture was vigorously stirred for 5 minutes then warmed to room temperature. EtOAc (10 mL) was added to the reaction, then concentrated in vacuo to remove MeOH. The residue was diluted with EtOAc (150 mL), washed with 1 M HCl (20 mL), brine (100 mL), dried over sodium sulfate, filtered, and concentrated in vacuo. The residue was purified by flash chromatography (loaded onto a 40 g SiO₂ cartridge with DCM, and eluted with 0-10% EtOAc in hexanes over a 20 min gradient, then 10-15% over a 10 minute gradient) to provide 6-bromofuro[2,3-b]pyrazine-2-carbaldehyde (1 g, 56%) as an off white solid ESI-MS m/z calc. 225.9378, found 227.2 (M+1)⁺; Retention time: 1.49 minutes. ¹H NMR (500 MHz, Chloroform-d) δ 10.20 (s, 1H), 8.91 (s, 1H), 7.16 (s, 1H). LC method H.

### Step 7: 3-[[4-[(2R)-2-[(6-Bromofuro[2,3-b]pyrazin-2-yl)methylamino]-3-(1-methylcyclopropyl)propoxy]-6-(2,6-dimethylphenyl)pyrimidin-2-yl]sulfamoyl]benzoic acid

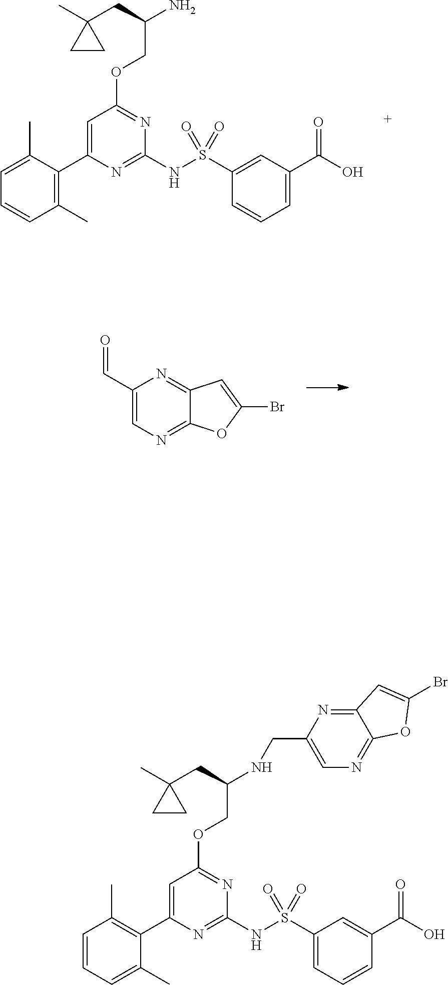 [SMILES: Cc1cccc(C)c1-c1cc(OC[C@H](N)CC2(C)CC2)nc(NS(=O)(=O)c2cccc(C(=O)O)c2)n1 ; O=Cc1cnc2oc(Br)cc2n1 ; Cc1cccc(C)c1-c1cc(OC[C@@H](CC2(C)CC2)NCc2cnc3oc(Br)cc3n2)nc(NS(=O)(=O)c2cccc(C(=O)O)c2)n1]

In a round bottom flask, 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (650 mg, 1.188 mmol) and 6-bromofuro[2,3-b]pyrazine-2-carbaldehyde (297 mg, 1.308 mmol) were combined in DCM (6.5 mL). Glacial acetic acid (134 μL, 2.356 mmol) was added followed by the addition of DIEA (620 μL, 3.559 mmol) at 0° C. After stirring at 0° C. for 15 minutes, sodium triacetoxyborohydride (1.26 g, 5.945 mmol) was added. Stirring was continued at 0° C. for 30 minutes. The reaction was quenched with the addition of methanol followed by aqueous HCl (1 M). It was then diluted with EtOAc (75 mL) and washed with aqueous HCl (1 M, 75 mL) and brine (75 mL). The organic layer was dried over sodium sulfate, filtered and concentrated under reduced pressure. The obtained white solid was triturated with iPrOH/EtOAc and hexanes. Solids were collected by vacuum filtration, rinsing with hexanes. This provided 3-[[4-[(2R)-2-[(6-bromofuro[2,3-b]pyrazin-2-yl)methylamino]-3-(1-methylcyclopropyl)propoxy]-6-(2,6-dimethylphenyl)pyrimidin-2-yl]sulfamoyl]benzoic acid (hydrochloride salt) (883 mg, 98%) as a white solid. ESI-MS m/z calc. 720.1366, found 721.4 (M+1)⁺; Retention time: 1.11 minutes. LC method A.

### Step 8: (11R)-12-[(6-Bromofuro[2,3-b]pyrazin-2-yl)methyl]-6-(2,6-dimethylphenyl)-11-[(I-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-254

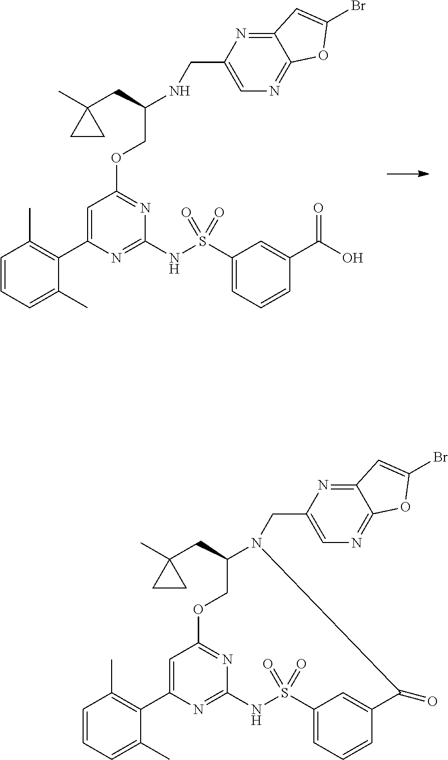 [SMILES: Cc1cccc(C)c1-c1cc(OC[C@@H](CC2(C)CC2)NCc2cnc3oc(Br)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(Br)cc3n1)[C@H](CC1(C)CC1)CO2]

In a round bottom flask, 3-[[4-[(2R)-2-[(6-bromofuro[2,3-b]pyrazin-2-yl)methylamino]-3-(1-methylcyclopropyl)propoxy]-6-(2,6-dimethylphenyl)pyrimidin-2-yl]sulfamoyl]benzoic acid (hydrochloride salt) (846 mg, 1.116 mmol) was combined with 2-chloro-4,6-dimethoxy-1,3,5-triazine (235 mg, 1.338 mmol) in DMF (12.5 mL). The solution was cooled to 0° C. before the addition of 4-methylmorpholine (610 μL, 5.548 mmol). The reaction mixture was allowed to slowly warm to room temperature and stirred for overnight. It was then diluted with EtOAc (75 mL) and washed with aqueous HCl (1 M, 75 mL) and brine (75 mL). The organic layer was dried over sodium sulfate, filtered and concentrated under reduced pressure. Purification by flash chromatography on silica gel (0-50% EtOAc/hexane gradient over 30 minutes) gave (11R)-12-[(6-bromofuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (513 mg, 65%) as a white solid. ¹H NMR (400 MHz, CDCl₃) δ 8.73 (s, 1H), 8.48 (s, 1H), 8.07 (d, J=1.4 Hz, 1H), 7.85 (d, J=1.4 Hz, 1H), 7.62 (t, J=7.8 Hz, 1H), 7.21 (t, J=7.6 Hz, 1H), 7.09-7.00 (m, 3H), 6.24 (s, 1H), 5.44 (dd, J=11.3, 4.3 Hz, 1H), 5.26 (d, J=14.9 Hz, 1H), 4.46-4.36 (m, 1H), 4.28-4.09 (m, 2H), 2.00 (s, 6H), 1.82 (d, J=14.9 Hz, 1H), 1.46 (dd, J=15.2, 10.0 Hz, 1H), 0.50 (s, 3H), 0.38-0.30 (m, 1H), 0.29-0.21 (m, 1H), 0.17-0.10 (m, 1H), 0.05-0.01 (m, 1H). ESI-MS m/z calc. 702.126, found 703.5 (M+1)⁺; Retention time: 1.68 minutes. LC method A.

### Step 9: (11R)-6-(2,6-Dimethylphenyl)-12-[(6-isopropenylfuro[2,3-b]pyrazin-2-yl)methyl]-11-[(I-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-255

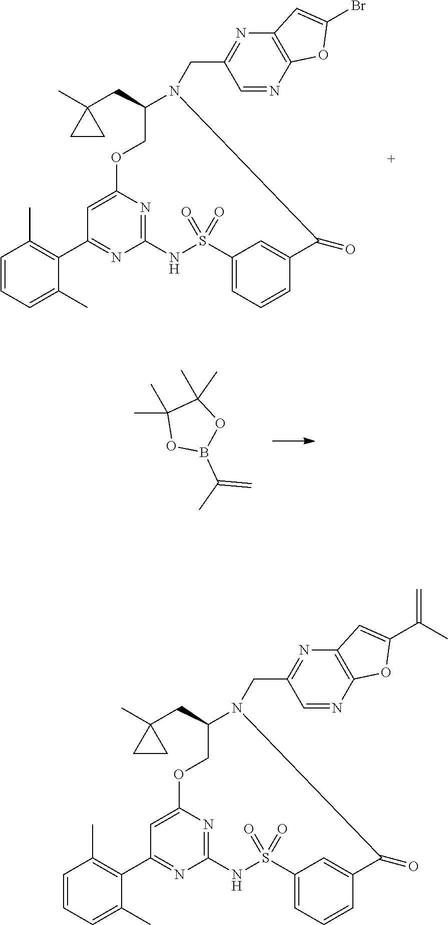 [SMILES: Cc1cccc(C)c1-c1cc2nc(n1)NS(=O)(=O)c1cccc(c1)C(=O)N(Cc1cnc3oc(Br)cc3n1)[C@H](CC1(C)CC1)CO2 ; C=C(C)B1OC(C)(C)C(C)(C)O1 ; C=C(C)c1cc2nc(CN3C(=O)c4cccc(c4)S(=O)(=O)Nc4nc(cc(-c5c(C)cccc5C)n4)OC[C@H]3CC3(C)CC3)cnc2o1]

A 5 mL microwave tube was charged with (11R)-12-[(6-bromofuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-254) (30 mg, 0.04264 mmol), 2-isopropenyl-4,4,5,5-tetramethyl-1,3,2-dioxaborolane (16 mg, 0.09521 mmol), potassium carbonate (25 mg, 0.1809 mmol) and Pd(dppf)Cl₂·DCM (5 mg, 0.006123 mmol), in that order. Then acetonitrile (450 μL) and water (150 μL) were added, and the tube was sparged with nitrogen for 3 min. The capped tube was stirred at 80° C. for 15 h (overnight). The reaction mixture was allowed to cool to room temperature and partitioned between EtOAc (15 mL) and 10% aqueous citric acid (5 mL). The organic layer was washed with brine (10 mL), dried over sodium sulfate, filtered, and concentrated under reduced pressure to dryness. The crude material was purified by reverse-phase HPLC (1-99% acetonitrile in water over 15 min, 5 mM HCl as modifier) to furnish product as a white solid. (11R)-6-(2,6-dimethylphenyl)-12-[(6-isopropenylfuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound I-255) (17 mg, 59%). ¹H NMR (400 MHz, MeOD) δ 8.78 (t, J=1.8 Hz, 1H), 8.39 (s, 1H), 8.06-7.98 (m, 1H), 7.79-7.72 (m, 1H), 7.68 (t, J=7.7 Hz, 1H), 7.27 (t, J=7.7 Hz, 1H), 7.14 (d, J=7.7 Hz, 2H), 7.09 (s, 1H), 6.29 (s, 1H), 5.99 (s, 1H), 5.60 (dd, J=10.6, 3.8 Hz, 1H), 5.44 (t, J=1.6 Hz, 1H), 5.14 (d, J=15.6 Hz, 1H), 4.52 (d, J=15.6 Hz, 1H), 4.47-4.36 (m, 1H), 4.32 (t, J=11.1 Hz, 1H), 2.20 (s, 3H), 2.10 (s, 6H), 1.83 (d, J=15.1 Hz, 1H), 1.59 (dd, J=15.3, 9.8 Hz, 1H), 0.52 (s, 3H), 0.40 (dt, J=9.7, 4.4 Hz, 1H), 0.26 (dt, J=9.5, 4.9 Hz, 1H), 0.16 (dt, J=9.3, 4.9 Hz, 1H), 0.07 (dt, J=9.6, 4.8 Hz, 1H). ESI-MS m/z calc. 664.24677, found 665.1 (M+1)⁺; Retention time: 1.91 minutes. LC method A.

### Step 10: (11R)-12-[[6-(1,2-Dihydroxy-1-methyl-ethyl)furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound IV-14), and (11R)-12-[[6-(1,2-dihydroxy-1-methyl-ethyl)furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound IV-15

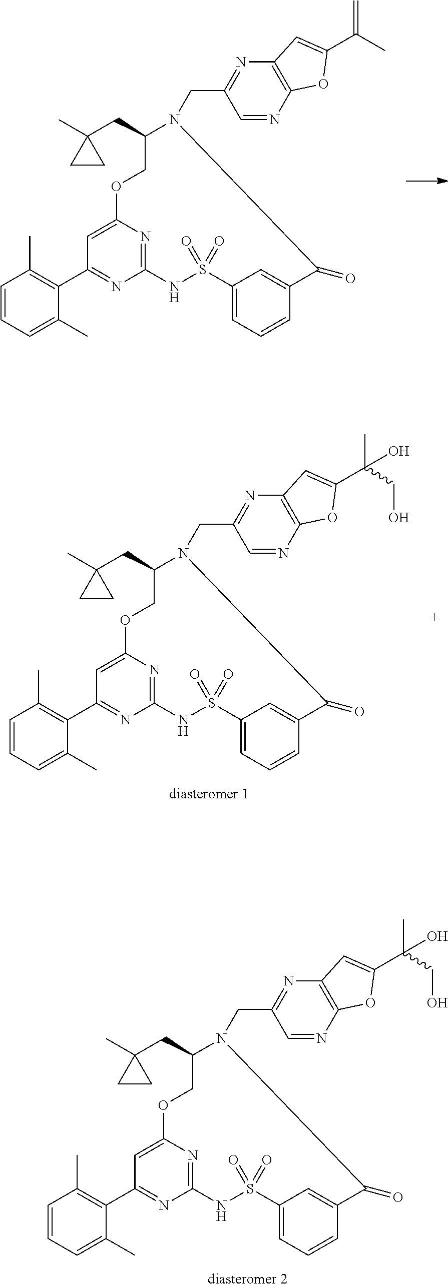 [SMILES: C=C(C)c1cc2nc(CN3C(=O)c4cccc(c4)S(=O)(=O)Nc4nc(cc(-c5c(C)cccc5C)n4)OC[C@H]3CC3(C)CC3)cnc2o1 ; Cc1cccc(C)c1-c1cc2nc(n1)NS(=O)(=O)c1cccc(c1)C(=O)N(Cc1cnc3oc(C(C)(O)CO)cc3n1)[C@H](CC1(C)CC1)CO2 ; Cc1cccc(C)c1-c1cc2nc(n1)NS(=O)(=O)c1cccc(c1)C(=O)N(Cc1cnc3oc(C(C)(O)CO)cc3n1)[C@H](CC1(C)CC1)CO2]

To a solution of (11R)-6-(2,6-dimethylphenyl)-12-[(6-isopropenylfuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (12 mg, 0.01805 mmol) in acetone (400 μL) and water (200 μL), were added 4-methylmorpholine N-oxide (15 mg, 0.1280 mmol) and osmium tetroxide (25 μL of 2.5% w/v, 0.002458 mmol), in that order, and the mixture was stirred under air at ambient temperature. After 1.5 h, the reaction turned to a clear solution. The reaction was concentrated to a light-yellow paste. The crude was purified via reverse-phase HPLC to furnish diastereomeric mixture as a white solid of (11R)-12-[[6-(1,2-dihydroxy-1-methyl-ethyl)furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (9 mg, 71%) ESI-MS m/z calc. 698.25226, found 699.2 (M+1)⁺; Retention time: 1.52 minutes. It was subjected to chiral SFC Purification [Instrument: Waters SFC Prep 100q (Perseus); Column: ChiralPak IC (21.2×250 mm, 5 μm); Temperature: 40° C.; Mode: Isocratic; Mobile Phase: 40% MeOH+20 mM NH₃; Flow: 70 mL/min; Concentration: 22.6 mg/mL (Methanol, no modifier); Injection Volume: 300 μL; Pressure: 205 bar; Wavelength: 210 nm; M/Z: 698.788] to furnish two diastereomers as white solids: Peak 1, diastereomer 1, (11R)-12-[[6-(1,2-dihydroxy-1-methyl-ethyl)furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (ammonium salt) (3.1 mg, 23%) ¹H NMR (400 MHz, MeOD) δ 8.74 (s, 1H), 8.39 (s, 1H), 8.08-7.95 (m, 1H), 7.72 (d, J=7.5 Hz, 1H), 7.65 (t, J=7.5 Hz, 1H), 7.32-7.20 (m, 1H), 7.12 (d, J=7.6 Hz, 2H), 7.05 (s, 1H), 6.24 (s, 1H), 5.57 (dd, J=10.9, 4.0 Hz, 1H), 5.13 (d, J=15.4 Hz, 1H), 4.51 (d, J=15.4 Hz, 1H), 4.45-4.41 (m, 1H), 4.31 (t, J=11.3 Hz, 1H), 3.81 (d, J=11.2 Hz, 1H), 3.76 (d, J=11.2 Hz, 1H), 2.09 (s, 6H), 1.84 (d, J=15.1 Hz, 1H), 1.60 (s, 3H), 1.59-1.53 (m, 1H), 0.50 (s, 3H), 0.39 (dt, J=9.4, 4.8 Hz, 1H), 0.25 (dt, J=9.7, 5.0 Hz, 1H), 0.14 (dt, J=9.2, 4.8 Hz, 1H), 0.05 (dt, J=9.7, 4.8 Hz, 1H). ESI-MS m/z calc. 698.25226, found 699.1 (M+1)⁺; Retention time: 1.52 minutes, and peak 2, diastereomer 2, (11R)-12-[[6-(1,2-dihydroxy-1-methyl-ethyl)furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (ammonium salt) (1.8 mg, 14%) ¹H NMR (400 MHz, MeOD) δ 8.75 (s, 1H), 8.39 (s, 1H), 8.03 (s, 1H), 7.71 (d, J=7.5 Hz, 1H), 7.65 (t, J=7.3 Hz, 1H), 7.22 (t, J=7.6 Hz, 1H), 7.10 (d, J=7.6 Hz, 2H), 7.05 (s, 1H), 6.19 (s, 1H), 5.61-5.53 (m, 1H), 5.13 (d, J=15.4 Hz, 1H), 4.51 (d, J=15.5 Hz, 1H), 4.48-4.40 (m, 1H), 4.27 (t, J=11.4 Hz, 1H), 3.84-3.74 (m, 2H), 2.07 (s, 6H), 1.84 (d, J=15.1 Hz, 1H), 1.60 (s, 3H), 1.58-1.53 (m, 1H), 0.49 (s, 3H), 0.42-0.34 (m, 1H), 0.28-0.21 (m, 1H), 0.17-0.09 (m, 1H), 0.08-0.02 (m, 1H). ESI-MS m/z calc. 698.25226, found 699.1 (M+1)⁺; Retention time: 1.52 minutes. LC method A.

### Example 107: Preparation of Compound IV-16

### Step 1: (11R)-6-(2,6-Dimethylphenyl)-12-[[6-(methoxymethyl)furo[2,3-b]pyrazin-2-yl]methyl]-11-[(I-methylcyclopropyl)methyl]-2,2-dioxo-9-oxa-2λ⁶-thia-3,5,12,19-tetraza-tricyclo[12.3.1.14,8]nonadeca-1(18), 4(19),5,7,14,16-hexaen-13-one (Compound IV-16

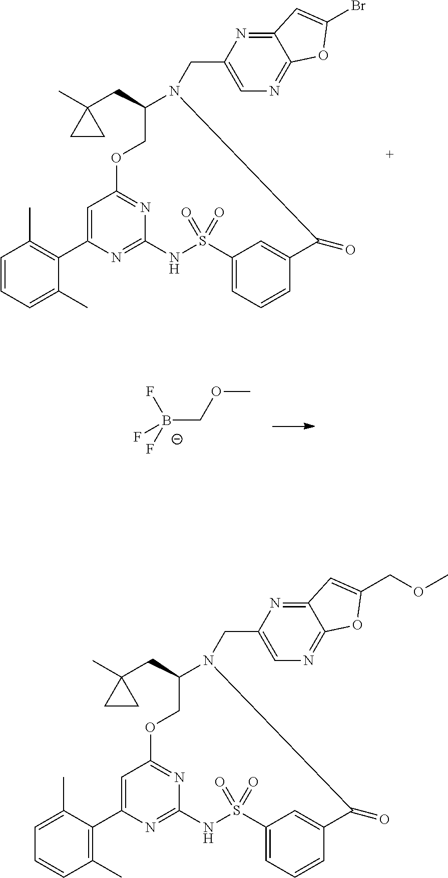

In a 4 mL vial, (11R)-12-[(6-bromofuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 0.04264 mmol), trifluoro(methoxymethyl)boranuide (potassium salt) (approximately 7.127 mg, 0.04690 mmol) and dicyclohexyl-[2-(2,6-diisopropoxyphenyl)phenyl]phosphane (approximately 1.990 mg, 0.004264 mmol) were combined in toluene (1 mL). Aqueous potassium carbonate (0.1 mL of 2 M, 0.2000 mmol) was added. After the addition of Pd(OAc)₂ (approximately 0.9573 mg, 0.004264 mmol), the vial was capped under nitrogen gas and stirred at 110° C. for 4 hours. Purification by reverse phase HPLC (10-99% acetonitrile/5 mM aqueous HCl over 15 minutes) gave (11R)-6-(2,6-dimethylphenyl)-12-[[6-(methoxymethyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (8.7 mg, 29%). ESI-MS m/z calc. 668.2417, found 669.5 (M+1)⁺; Retention time: 1.56 minutes; LC method A.

### Example 108: Preparation of Compound IV-20 and Compound IV-21

### Step 1: 2-Amino-3,3-di(cyclobutyl)propan-1-ol

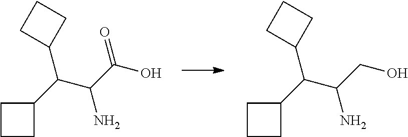 [SMILES: NC(C(=O)O)C(C1CCC1)C1CCC1 ; NC(CO)C(C1CCC1)C1CCC1]

To a solution of 2-amino-3,3-di(cyclobutyl)propanoic acid (hydrochloride salt) (1.0 g, 4.278 mmol) in tetrahydrofuran (43 mL) was added LAH (437.2 mg, 12.89 mmol) portion-wise at 0° C. The reaction was allowed to warm to room temperature and stirred for 16 h. The reaction mixture was cooled to 0° C. and quenched with water (0.44 mL). After bubbling had ceased, 15% NaOH (0.44 mL) was slowly added followed by addition of water (1.3 mL). The reaction mixture was allowed to warm to room temperature and stirred for 15 minutes. Magnesium sulfate was then added, and the mixture was stirred for 15 minutes. The reaction mixture was then filtered through celite and rinsed with EtOAc. The filtrate was concentrated under reduced pressure to provide 2-amino-3,3-di(cyclobutyl)propan-1-ol (225.4 mg, 29%). ESI-MS m/z calc. 183.16231, found 184.1 (M+1)⁺; Retention time: 0.71 minutes. LC method A.

### Step 2: 3-[[4-[2-Amino-3,3-di(cyclobutyl)propoxy]-6-(2,6-dimethylphenyl)pyrimidin-2-yl]sulfamoyl]benzoic acid

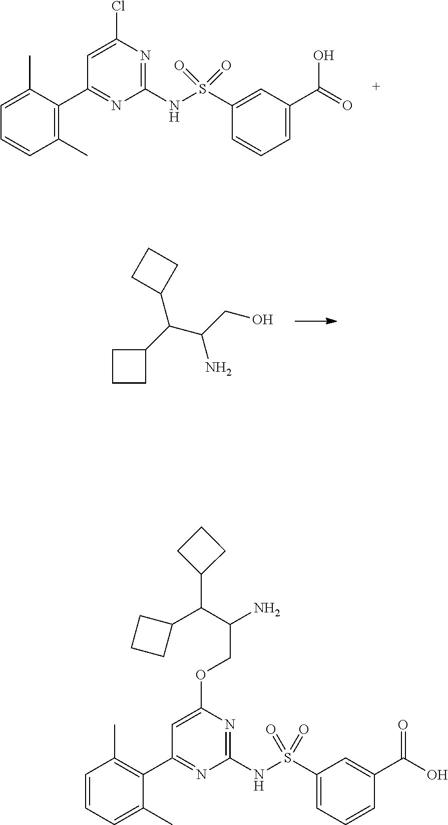 [SMILES: Cc1cccc(C)c1-c1cc(Cl)nc(NS(=O)(=O)c2cccc(C(=O)O)c2)n1 ; NC(CO)C(C1CCC1)C1CCC1 ; Cc1cccc(C)c1-c1cc(OCC(N)C(C2CCC2)C2CCC2)nc(NS(=O)(=O)c2cccc(C(=O)O)c2)n1]

3-[[4-Chloro-6-(2,6-dimethylphenyl)pyrimidin-2-yl]sulfamoyl]benzoic acid (200 mg, 0.4786 mmol) and 2-amino-3,3-di(cyclobutyl)propan-1-ol (108.1 mg, 0.5898 mmol) were combined under nitrogen in anhydrous THE (2.4 mL). To the solution sodium tert-butoxide (186.8 mg, 1.944 mmol) was added in one portion resulting in a slightly exothermic reaction. The mixture was stirred at room temperature for 30 minutes. The reaction was diluted with ethyl acetate (20 mL), 1 M HCl (20 mL), and brine (20 mL), and the resulting two phases were separated. The aqueous phase was further extracted with EtOAc (3×10 mL). The combined organic extracts were dried over magnesium sulfate, filtered, and concentrated under reduced pressure to give 3-[[4-[2-amino-3,3-di(cyclobutyl)propoxy]-6-(2,6-dimethylphenyl)pyrimidin-2-yl]sulfamoyl]benzoic acid (hydrochloride salt) (286.3 mg, 100%) as a orange solid. ESI-MS m/z calc. 564.24066, found 565.4 (M+1)⁺; Retention time: 1.2 minutes. LC method A.

### Step 3: 3-[[4-[3,3-di(Cyclobutyl)-2-[[5-methyl-6-(1-methylcyclopropyl)pyrrolo[2,3-b]pyrazin-3-yl]methylamino]propoxy]-6-(2,6-dimethylphenyl)pyrimidin-2-yl]sulfamoyl]benzoic acid

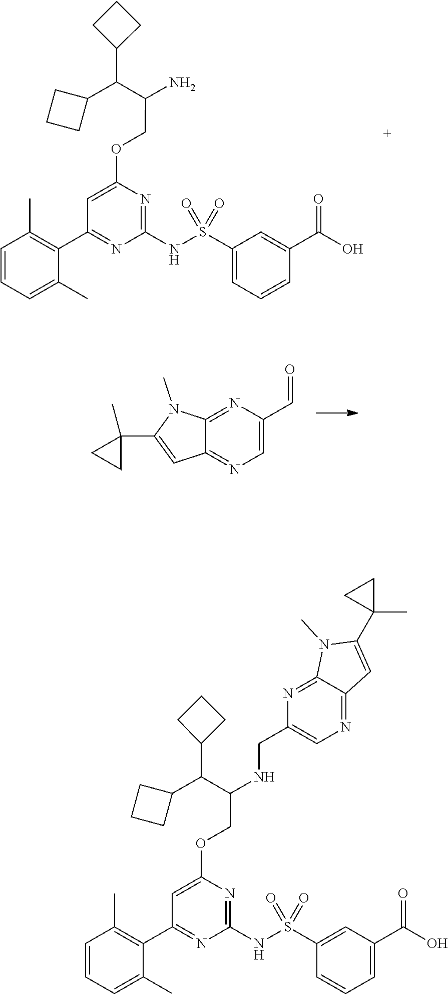 [SMILES: Cc1cccc(C)c1-c1cc(OCC(N)C(C2CCC2)C2CCC2)nc(NS(=O)(=O)c2cccc(C(=O)O)c2)n1 ; Cn1c(C2(C)CC2)cc2ncc(C=O)nc21 ; Cc1cccc(C)c1-c1cc(OCC(NCc2cnc3cc(C4(C)CC4)n(C)c3n2)C(C2CCC2)C2CCC2)nc(NS(=O)(=O)c2cccc(C(=O)O)c2)n1]

A 4 mL vial was charged under nitrogen with 3-[[4-[2-amino-3,3-di(cyclobutyl)propoxy]-6-(2,6-dimethylphenyl)pyrimidin-2-yl]sulfamoyl]benzoic acid (hydrochloride salt) (80 mg, 0.1331 mmol), 5-methyl-6-(1-methylcyclopropyl)pyrrolo[2,3-b]pyrazine-3-carbaldehyde (31.9 mg, 0.1482 mmol), anhydrous DCM (0.67 mL), and acetic acid (15.2 μL, 0.2673 mmol). The mixture was cooled down in an ice bath. DIPEA (70.0 μL, 0.4019 mmol) was added, followed by sodium triacetoxyborohydride (145.2 mg, 0.6851 mmol), and the reaction was vigorously stirred at 0° C. for 1 h. The reaction was quenched with 3 N aqueous HCl, diluted with MeOH (0.5 mL) and DMSO (0.5 mL), and the resulting solution was filtered. Purification by reverse phase HPLC (1-99% acetonitrile/5 mM aqueous HCl over 15 min) provided 3-[[4-[3,3-di(cyclobutyl)-2-[[5-methyl-6-(1-methylcyclopropyl)pyrrolo[2,3-b]pyrazin-3-yl]methylamino]propoxy]-6-(2,6-dimethylphenyl)pyrimidin-2-yl]sulfamoyl]benzoic acid (hydrochloride salt) (44.0 mg, 41%) as a white solid. ESI-MS m/z calc. 763.35156, found 764.4 (M+1)⁺; Retention time: 1.61 minutes. LC method A.

### Step 4: 11-[di(Cyclobutyl)methyl]-6-(2,6-dimethylphenyl)-12-[[5-methyl-6-(1-methylcyclopropyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one, isomer 1 (Compound IV-20), and 11-[di(cyclobutyl)methyl]-6-(2,6-dimethylphenyl)-12-[[5-methyl-6-(1-methylcyclopropyl)pyrrolo[2,3-b]pyrazin-3-yl]methyl]-2,2-dioxo-9-oxa-226-thia-3,5,12,19-tetrazatricyclo[12.3.1.14,8]nonadeca-1(18),4(19),5,7,14,16-hexaen-13-one, isomer 2 (Compound IV-21

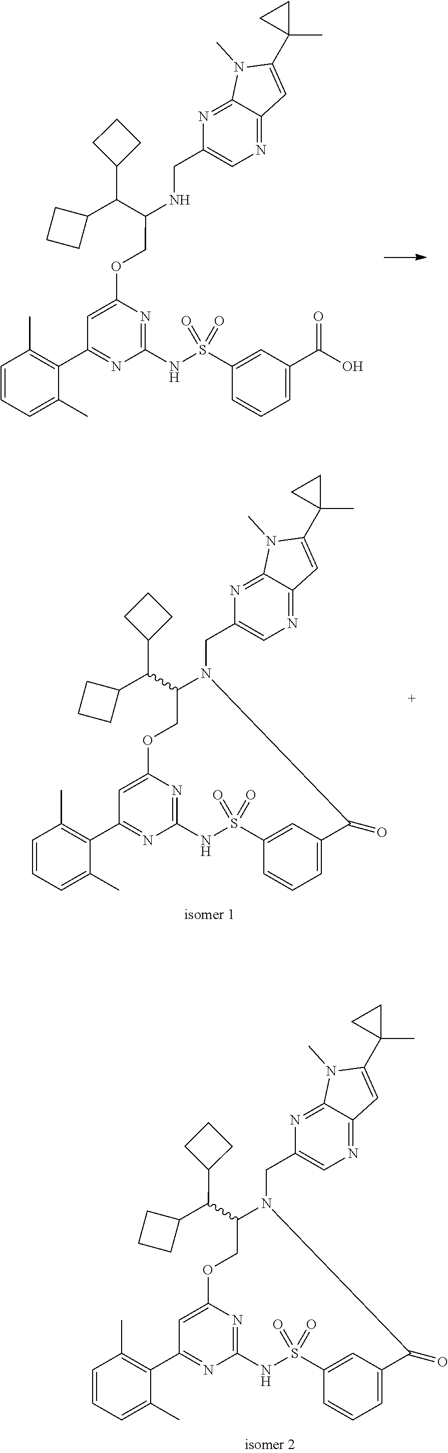 [SMILES: Cc1cccc(C)c1-c1cc(OCC(NCc2cnc3cc(C4(C)CC4)n(C)c3n2)C(C2CCC2)C2CCC2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3cc(C4(C)CC4)n(C)c3n1)C(C(C1CCC1)C1CCC1)CO2 ; Cc1cccc(C)c1-c1cc2nc(n1)NS(=O)(=O)c1cccc(c1)C(=O)N(Cc1cnc3cc(C4(C)CC4)n(C)c3n1)C(C(C1CCC1)C1CCC1)CO2]

3-[[4-[3,3-di(Cyclobutyl)-2-[[5-methyl-6-(1-methylcyclopropyl)pyrrolo[2,3-b]pyrazin-3-yl]methylamino]propoxy]-6-(2,6-dimethylphenyl)pyrimidin-2-yl]sulfamoyl]benzoic acid (hydrochloride salt) (44 mg, 0.05497 mmol) was combined with CDMT (16.1 mg, 0.09170 mmol) in DMF (5.5 mL) and cooled to 0° C. N-methylmorpholine (18.2 μL, 0.1655 mmol) was added by syringe and the reaction was stirred at 0° C. for 30 minutes. The ice bath was then removed and stirring was continued for an additional 24 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give 20 mg of a white solid that was submitted for SFC separation. The mixture was subjected to chiral SFC (ChiralCel OD (21.2×250 mm, 5 m column; 40° C., isocratic mode, 28% MeOH+20 mM NH₃, 70 mL/min, concentration 19 mg/mL (methanol), injection volume 500 μL, pressure 162 bar, wavelength 210 nm). For each separated isomer, the solvent was evaporated. The resulting solid was dissolved in DMSO (1 mL) and purified by reverse phase HPLC (1-99% acetonitrile/5 mM aqueous HCl over 15 min) to give as solids: Isomer 1, SFC peak 1, 11-[di(cyclobutyl)methyl]-6-(2,6-dimethylphenyl)-12-[[5-methyl-6-(1-methylcyclopropyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (1.9 mg, 9%) ¹H NMR (400 MHz, CDCl₃) δ 9.02 (s, 1H), 8.46 (s, 1H), 7.96 (d, J=7.9 Hz, 1H), 7.90 (d, J=7.6 Hz, 1H), 7.60 (t, J=7.8 Hz, 1H), 7.23 (d, J=7.6 Hz, 1H), 7.10 (d, J=7.6 Hz, 2H), 6.55 (s, 1H), 6.40 (s, 1H), 5.68 (d, J=8.0 Hz, 1H), 5.49 (d, J=15.5 Hz, 1H), 4.49 (d, J=15.5 Hz, 1H), 4.43-4.30 (m, 2H), 4.02 (s, 3H), 2.46-2.35 (m, 1H), 2.29-2.19 (m, 1H), 2.09 (s, 6H), 1.98-1.87 (m, 3H), 1.77-1.68 (m, 3H), 1.67-1.57 (m, 3H), 1.51-1.46 (m, 1H), 1.44 (s, 3H), 1.26-1.15 (m, 2H), 1.02-0.94 (m, 2H), 0.93-0.81 (m, 3H). ESI-MS m/z calc. 745.341, found 746.2 (M+1)⁺; Retention time: 2.03 minutes. and Isomer 2, SFC peak 2, 11-[di(cyclobutyl)methyl]-6-(2,6-dimethylphenyl)-12-[[5-methyl-6-(1-methylcyclopropyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (2.1 mg, 10%) ¹H NMR (400 MHz, CDCl₃) δ 9.01 (s, 1H), 8.46 (s, 1H), 8.00 (d, J=7.9 Hz, 1H), 7.91 (d, J=7.6 Hz, 1H), 7.62 (t, J=7.8 Hz, 1H), 7.23 (d, J=7.6 Hz, 1H), 7.10 (d, J=7.6 Hz, 2H), 6.59 (s, 1H), 6.41 (s, 1H), 5.68 (d, J=8.1 Hz, 1H), 5.48 (d, J=15.4 Hz, 1H), 4.49 (d, J=15.5 Hz, 1H), 4.43-4.30 (m, 2H), 4.03 (s, 3H), 2.48-2.33 (m, 1H), 2.29-2.20 (m, 1H), 2.09 (s, 6H), 2.06-1.99 (m, 2H), 1.87-1.79 (m, 2H), 1.77-1.68 (m, 3H), 1.67-1.60 (m, 3H), 1.50-1.47 (m, 1H), 1.44 (s, 3H), 1.24-1.15 (m, 1H), 1.03-0.94 (m, 2H), 0.94-0.82 (m, 3H). ESI-MS m/z calc. 745.341, found 746.2 (M+1)⁺; Retention time: 2.03 minutes. LC method A.

### Example 109: Preparation of Compound IV-22, Compound IV-23 and Compound IV-26

### Step 1: (11R)-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-22), (11R)-6-(2,6-dimethylphenyl)-12-[[6-(1-hydroxy-1-methyl-ethyl)furo[2,3-b]pyrazin-2-yl]methyl]-11-[(1-methylcyclopropyl)-methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-23), (11R)-6-(2,6-dimethylphenyl)-12-(furo[2,3-b]pyrazin-2-ylmethyl)-11-[(1-methylcyclopropyl)methyl]-2,2-dioxo-9-oxa-226-thia-3,5,12,19-tetrazatricyclo[12.3.1.14,8]nonadeca-1(18),4(19),5,7,14,16-hexaen-13-one (Compound IV-24

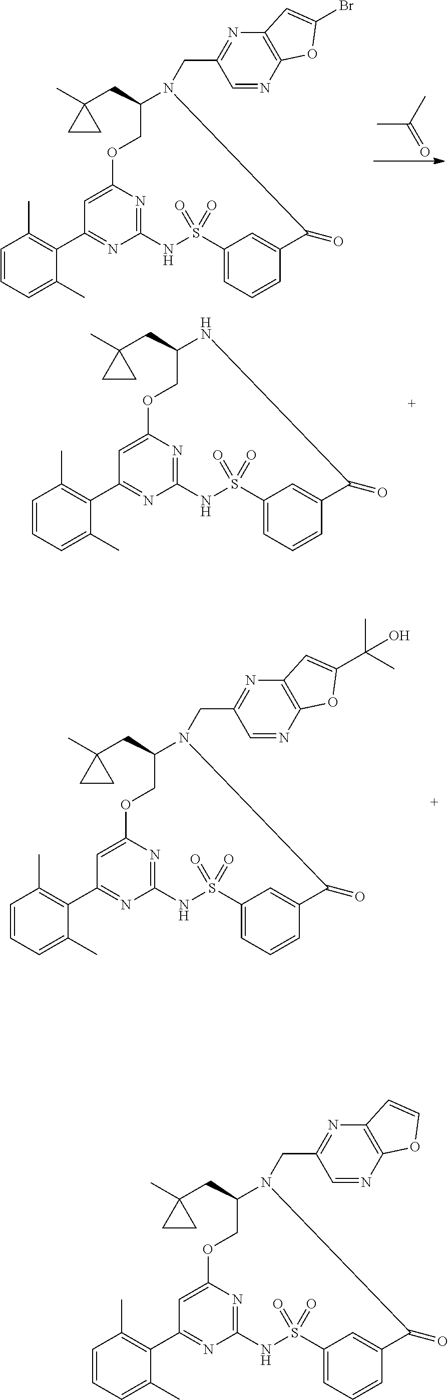

To a stirred solution of (11R)-12-[(6-bromofuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (70 mg, 0.09849 mmol) in anhydrous tetrahydrofuran (1.2 mL) was added n-butyllithium (2.5 M in hexanes) (100 μL of 2.5 M, 0.2500 mmol) over 1 min at −78° C. (dry ice-acetone bath). After stirring the resulting yellow heterogeneous mixture at that temperature for 30 min, a solution of acetone (20 μL, 0.2724 mmol) in anhydrous tetrahydrofuran (0.2 mL) was added dropwise over 2 min. Then stirred for 15 min and the dry ice-acetone bath was removed and let the reaction warm to room temperature and stirred for 1 h at room temperature. The reaction was quenched with glacial acetic acid (70 μL, 1.231 mmol) and concentrated under reduced pressure. The residue was taken up in DMSO (2 mL) and purified by preparative reverse phase chromatography (10-99% acetonitrile in water over 20 min, no modifier). The various fractions were collected and dried in to furnish four compounds as white solids. The first one to elute from the column was (11R)-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (3.8 mg, 8%) ¹H NMR (400 MHz, MeOD) δ 8.67 (t, J=1.8 Hz, 1H), 8.05 (dt, J=7.7, 1.6 Hz, 1H), 7.75 (dt, J=7.6, 1.5 Hz, 1H), 7.70 (t, J=7.7 Hz, 1H), 7.27 (t, J=7.6 Hz, 1H), 7.14 (d, J=7.7 Hz, 2H), 6.29 (s, 1H), 5.33 (dd, J=10.8, 3.9 Hz, 1H), 3.94 (t, J=11.2 Hz, 1H), 3.75-3.65 (m, 1H), 2.10 (s, 6H), 1.85 (d, J=14.7 Hz, 1H), 1.18 (dd, J=14.6, 10.4 Hz, 1H), 0.54 (s, 3H), 0.50 (dt, J=9.6, 4.9 Hz, 1H), 0.24 (dt, J=9.7, 4.9 Hz, 1H), 0.15-0.08 (m, 1H), 0.05-0.01 (m, 1H). ESI-MS m/z calc. 492.18314, found 493.0 (M+1)⁺; Retention time: 1.43 minutes. The second component was (11R)-6-(2,6-dimethylphenyl)-12-[[6-(1-hydroxy-1-methyl-ethyl)furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (4.8 mg, 7%) ¹H NMR (400 MHz, MeOD) δ 8.75 (t, J=1.8 Hz, 1H), 8.38 (s, 1H), 8.02 (d, J=7.7 Hz, 1H), 7.78-7.71 (m, 1H), 7.67 (t, J=7.7 Hz, 1H), 7.27 (t, J=7.6 Hz, 1H), 7.14 (d, J=7.7 Hz, 2H), 6.96 (s, 1H), 6.28 (s, 1H), 5.59 (dd, J=10.4, 3.7 Hz, 1H), 5.13 (d, J=15.5 Hz, 1H), 4.52 (d, J=15.5 Hz, 1H), 4.45-4.36 (m, 1H), 4.32 (t, J=11.1 Hz, 1H), 2.10 (s, 6H), 1.83 (d, J=15.2 Hz, 1H), 1.65 (s, 6H), 1.58 (dd, J=15.3, 9.6 Hz, 1H), 0.51 (s, 3H), 0.40 (dt, J=9.7, 4.6 Hz, 1H), 0.26 (dt, J=9.7, 4.9 Hz, 1H), 0.15 (dt, J=9.3, 4.8 Hz, 1H), 0.06 (dt, J=9.6, 4.9 Hz, 1H). ESI-MS m/z calc. 682.2573, found 683.1 (M+1)⁺; Retention time: 165.0 minutes. The third compound eluted was product (11R)-6-(2,6-dimethylphenyl)-12-(furo[2,3-b]pyrazin-2-ylmethyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (9 mg, 14%) ¹H NMR (400 MHz, MeOD) δ 8.77 (t, J=1.8 Hz, 1H), 8.47 (s, 1H), 8.24 (d, J=2.6 Hz, 1H), 8.03 (d, J=7.7 Hz, 1H), 7.75 (d, J=7.6 Hz, 1H), 7.68 (t, J=7.7 Hz, 1H), 7.27 (t, J=7.6 Hz, 1H), 7.19-7.11 (m, 3H), 6.29 (s, 1H), 5.60 (dd, J=10.4, 3.7 Hz, 1H), 5.16 (d, J=15.6 Hz, 1H), 4.55 (d, J=15.5 Hz, 1H), 4.46-4.38 (m, 1H), 4.37-4.29 (m, 1H), 2.10 (s, 6H), 1.84 (d, J=15.1 Hz, 1H), 1.59 (dd, J=15.2, 9.7 Hz, 1H), 0.51 (s, 3H), 0.41 (dt, J=9.4, 4.7 Hz, 1H), 0.26 (dt, J=9.7, 4.9 Hz, 1H), 0.16 (dt, J=9.2, 4.8 Hz, 1H), 0.07 (dt, J=9.7, 4.9 Hz, 1H). ESI-MS m/z calc. 624.2155, found 625.1 (M+1)⁺; Retention time: 1.66 minutes. A fourth compound was isolated impure and kept aside. (11R)-12-[[6-(1,3-dihydroxy-1,3-dimethyl-butyl)furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (3 mg, 4%) ESI-MS m/z calc. 740.2992, found 741.2 (M+1)⁺; Retention time: 1.66 minutes. LC method A.

### Example 110: Preparation of Compound IV-17 and Compound IV-24

### Step 1: (11R)-6-(2,6-Dimethylphenyl)-11-[(1-methylcyclopropyl)methyl]-12-[[6-(2-methyl-prop-1-enyl)furo[2,3-b]pyrazin-2-yl]methyl]-2,2-dioxo-9-oxa-2λ⁶-thia-3,5,12,19-tetraza-tricyclo[12.3.1.14,8]nonadeca-1(18),4(19),5,7,14,16-hexaen-13-one (Compound IV-17

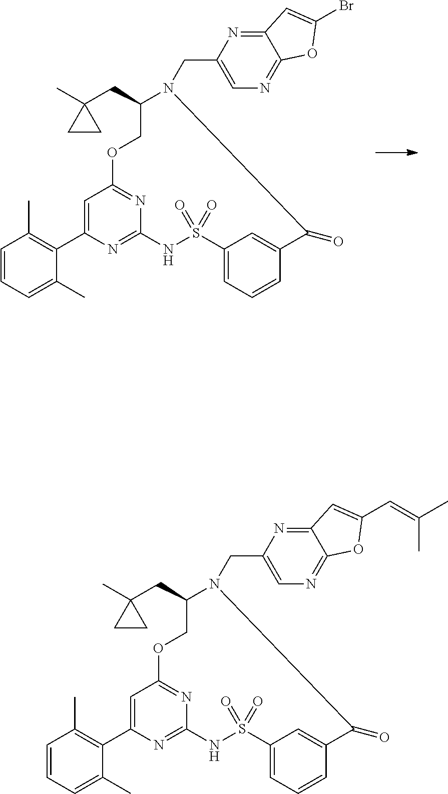 [SMILES: Cc1cccc(C)c1-c1cc2nc(n1)NS(=O)(=O)c1cccc(c1)C(=O)N(Cc1cnc3oc(Br)cc3n1)[C@H](CC1(C)CC1)CO2 ; CC(C)=Cc1cc2nc(CN3C(=O)c4cccc(c4)S(=O)(=O)Nc4nc(cc(-c5c(C)cccc5C)n4)OC[C@H]3CC3(C)CC3)cnc2o1]

In a 4 mL vial, (11R)-12-[(6-bromofuro[2,3-b]pyrazin-2-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (40 mg, 0.05685 mmol) and trifluoro(2-methylprop-1-enyl)boranuide (potassium salt) (12 mg, 0.07407 mmol) were combined in ethanol (800 μL). triethylamine (50 μL, 0.3587 mmol) was added followed by the addition of [1,1-bis(diphenylphosphino)ferrocene]dichloropalladium, complex with dichloromethane (5 mg, 0.006123 mmol). The vial was capped under nitrogen gas and stirred at 80° C. for 1.5 hours. Purification by reverse phase HPLC (10-99% acetonitrile/5 mM aqueous HCl over 15 minutes) provided (11R)-6-(2,6-dimethylphenyl)-11-[(1-methylcyclopropyl)methyl]-12-[[6-(2-methylprop-1-enyl)furo[2,3-b]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 38%) as a white solid. ESI-MS m/z calc. 678.26245, found 679.5 (M+1)⁺; Retention time: 1.87 minutes.; LC method A.

### Step 2: (11R)-6-(2,6-Dimethylphenyl)-12-[(6-isobutylfuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-24

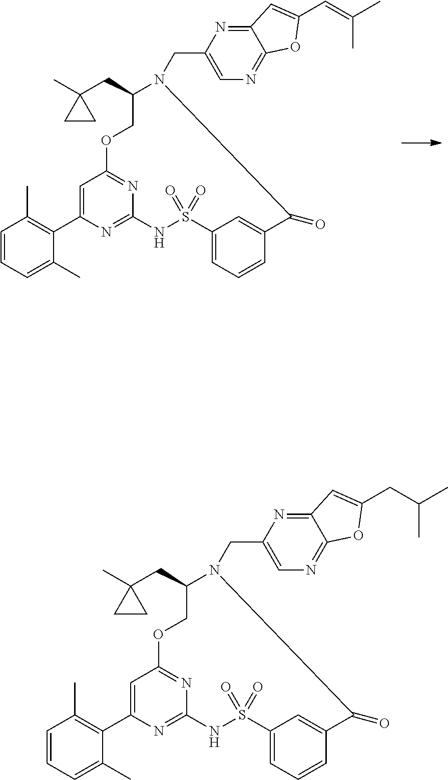 [SMILES: CC(C)=Cc1cc2nc(CN3C(=O)c4cccc(c4)S(=O)(=O)Nc4nc(cc(-c5c(C)cccc5C)n4)OC[C@H]3CC3(C)CC3)cnc2o1 ; Cc1cccc(C)c1-c1cc2nc(n1)NS(=O)(=O)c1cccc(c1)C(=O)N(Cc1cnc3oc(CC(C)C)cc3n1)[C@H](CC1(C)CC1)CO2]

In a round bottom flask, (11R)-6-(2,6-dimethylphenyl)-11-[(1-methylcyclopropyl)methyl]-12-[[6-(2-methylprop-1-enyl)furo[2,3-b]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (12 mg, 0.01768 mmol) was dissolved in ethanol (6 mL) and DCM (1 mL). Under nitrogen, palladium (5 mg, 0.004698 mmol) (10 wt % on carbon, degussa, wet) was added. The reaction mixture was stirred under a balloon of hydrogen gas overnight. Purification by reverse phase HPLC (10-99% acetonitrile/5 mM aqueous HCl over 15 minutes) provided (11R)-6-(2,6-dimethylphenyl)-12-[(6-isobutylfuro[2,3-b]pyrazin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (5 mg, 42%) as a white solid. ¹H NMR (400 MHz, CDCl₃) δ 8.74 (s, 1H), 8.41 (s, 1H), 8.06 (d, J=1.5 Hz, 1H), 7.86 (d, J=1.4 Hz, 1H), 7.62 (t, J=7.8 Hz, 1H), 7.21 (t, J=7.6 Hz, 1H), 7.05 (d, J=7.6 Hz, 2H), 6.65 (s, 1H), 6.24 (s, 1H), 5.41 (dd, J=11.2, 4.2 Hz, 1H), 5.33-5.25 (m, 1H), 4.46-4.37 (m, 1H), 4.28 (t, J=11.5 Hz, 1H), 4.16 (d, J=14.8 Hz, 1H), 2.74 (d, J=7.1 Hz, 2H), 2.20 (hept, J=6.7 Hz, 1H), 2.00 (s, 6H), 1.81 (d, J=1.8 Hz, 1H), 1.48 (dd, J=15.1, 10.0 Hz, 1H), 1.03 (d, J=1.1 Hz, 6H), 0.49 (s, 3H), 0.38-0.32 (m, 1H), 0.28-0.22 (m, 1H), 0.16-0.10 (m, 1H), 0.05-0.00 (m, 1H). ESI-MS m/z calc. 680.2781, found 681.5 (M+1)⁺; Retention time: 1.87 minutes. LC method A.

### Example 111: Preparation of Compound IV-35

### Step 1: 5-Bromo-2-chloro-N-methyl-pyrimidin-4-amine

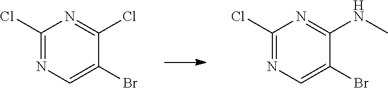

To a solution of 5-bromo-2,4-dichloro-pyrimidine (16 g, 70.214 mmol) in methanol (130 mL) was added dropwise methylamine in ethanol (461.54 mg, 1.85 mL of 33% w/w, 4.9041 mmol) at 0° C. The mixture was stirred for 20 minutes at 0° C. and for 3 hours at room temperature. The solvent was evaporated, and the residue was solubilized with ethyl acetate (100 mL). The organic layer was washed with a saturated aqueous solution of sodium bicarbonate (2×100 mL) and a saturated aqueous solution of NaCl (100 mL). The organic layer was dried over sodium sulfate, filtered, concentrated under reduced pressure, and recrystallized in heptane and ethyl acetate. The solid was filtered on Buchner funnel, rinsed with heptane and dried under high vacuum to give 5-bromo-2-chloro-N-methyl-pyrimidin-4-amine (13.7 g, 86%) as a white crystalline solid ¹H NMR (400 MHz, CDCl₃) δ 8.10 (s, 1H), 5.62 (br. s, 1H), 3.09 (d, J=4.9 Hz, 3H) ESI-MS m/z calc. 220.9355, found 222.0 (M+1)⁺; Retention time: 1.51 minutes. LC method I.

### Step 2: 2-Chloro-5-(3,3-dimethylbut-1-ynyl)-N-methyl-pyrimidin-4-amine

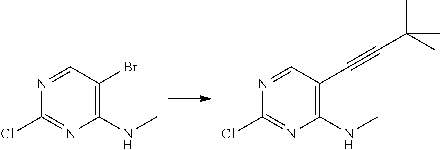

5-Bromo-2-chloro-N-methyl-pyrimidin-4-amine (8.459 g, 36.122 mmol) was solubilized in in THE (80 mL) and Triethylamine (80 mL) was added dropwise to the solution. CuI (340 mg, 1.7852 mmol) and bis(triphenylphosphine)palladium(II)chloride (1.27 g, 1.8094 mmol) were added. The mixture was purged with nitrogen for 5 minutes and 3,3-dimethyl-1-butyne (3.6018 g, 5.4 mL, 43.848 mmol) was added. The mixture was stirred for 1.5 hours at 50° C. The reaction was diluted in ethyl acetate (200 mL) and filtered over a pad of celite. Then, the celite was rinsed with ethyl acetate (100 mL). The filtrate was concentrated under reduced pressure and purified by reverse flash chromatography (gradient: acetonitrile in aqueous formic acid solution 0.1%, 5 to 80%, 12 CV) to afford 2-chloro-5-(3,3-dimethylbut-1-ynyl)-N-methyl-pyrimidin-4-amine (5.725 g, 57%) ¹H NMR (400 MHz, CDCl₃) δ 8.00 (s, 1H), 5.54 (br. s, 1H), 3.08 (d, J=4.9 Hz, 3H), 1.33 (s, 9H) as a yellow solid ESI-MS m/z calc. 223.0876, found 224.2 (M+1)⁺; Retention time: 1.88 minutes. LC method I.

### Step 3: 6-tert-Butyl-2-chloro-7-methyl-pyrrolo[2,3-d]pyrimidine

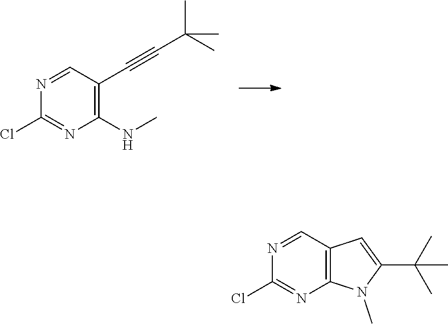

To a solution of 2-chloro-5-(3,3-dimethylbut-1-ynyl)-N-methyl-pyrimidin-4-amine (5.725 g, 20.422 mmol) in THE (140 mL) was added TBAF in THE (61 mL of 1 M, 61.000 mmol) and the mixture was stirred overnight at 65° C. The solvent was removed under reduced pressure and the residue was dissolved in EtOAc (400 mL) and the mixture was washed with water (2×200 mL) and brine (2×300 mL). The organic phase was dried over sodium sulfate, filtered, and concentrated under reduced pressure. Then, the crude product was purified by reverse flash chromatography on silica gel (gradient: 0 to 80% acetonitrile in aqueous formic acid solution, 12 CV). The pure fractions were concentrated under reduce pressure. The aqueous phase was extracted with EtOAc (50 mL). Organic layer was washed with sodium bicarbonate (3×100 mL) and brine (100 mL). Then, the organic layer was dried under sodium sulfate and evaporated under reduced pressure to afford 6-tert-butyl-2-chloro-7-methyl-pyrrolo[2,3-d]pyrimidine (4.166 g, 91%) as a white solid ¹H NMR (400 MHz, CDCl₃) δ 8.64 (s, 1H), 6.32 (s, 1H), 3.97 (s, 3H), 1.48 (s, 9H) ESI-MS m/z calc. 223.0876, found 220.2 (M+1)⁺; Retention time: 1.25 minutes. LC method I.

### Step 4: Methyl 6-tert-butyl-7-methyl-pyrrolo[2,3-d]pyrimidine-2-carboxylate

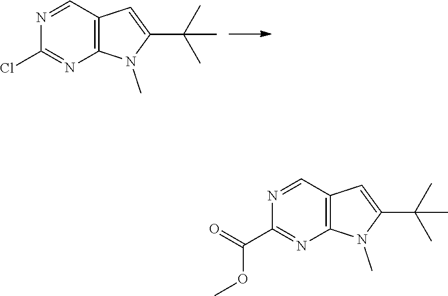 [SMILES: Cn1c(C(C)(C)C)cc2cnc(Cl)nc21 ; COC(=O)c1ncc2cc(C(C)(C)C)n(C)c2n1]

To a stirred solution of 6-tert-butyl-2-chloro-7-methyl-pyrrolo[2,3-d]pyrimidine (4.166 g, 18.604 mmol) and triethylamine (5.8080 g, 8 mL, 57.397 mmol) in methanol (45 mL) under nitrogen atmosphere was added 1,1′-bis(diphenylphosphino)ferrocene palladium(II) chloride, complex with dichloromethane (3.34 g, 4.0899 mmol). Nitrogen was bubbled for 15 minutes to the mixture and the reaction was stirred overnight at 100° C. under 60 psi of carbon monoxide. The reaction was then cooled down to room temperature and concentrated under reduced pressure. The crude was purified by flash chromatography on silica-gel (gradient: 5 to 60% ethyl acetate in heptanes; 15 CV) and the volatiles were concentrated under reduced pressure to afford methyl 6-tert-butyl-7-methyl-pyrrolo[2,3-d]pyrimidine-2-carboxylate (3.05 g, 66%) as a white solid ¹H NMR (400 MHz, CDCl₃) δ 8.95 (s, 1H), 6.45 (s, 1H), 4.10 (s, 3H), 4.07 (s, 3H), 1.51 (s, 9H) ESI-MS m/z calc. 247.1321, found 248.2 (M+1)⁺; Retention time: 1.6 minutes. LC method I.

### Step 5: (6-tert-Butyl-7-methyl-pyrrolo[2,3-d]pyrimidin-2-yl)methanol

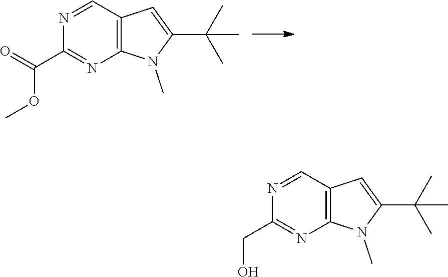 [SMILES: COC(=O)c1ncc2cc(C(C)(C)C)n(C)c2n1 ; Cn1c(C(C)(C)C)cc2cnc(CO)nc21]

To a solution of methyl 6-tert-butyl-7-methyl-pyrrolo[2,3-d]pyrimidine-2-carboxylate (3.05 g, 12.260 mmol) in MeOH (60 mL) was added NaBH₄ (2.32 g, 61.323 mmol) at 0° C. After one hour of stirring, NaBH₄ (578 mg, 15.278 mmol) was added. After two hours, NaBH₄ (584 mg, 15.436 mmol) was added. After three hours, NaBH₄ (574 mg, 15.172 mmol) was added. After four hours, NaBH₄ (587 mg, 15.516 mmol) was added. The reaction was stirred at room temperature for 24 hours. The mixture was quenched with water (200 mL), concentrated under reduce pressure and the residue was purified by flash chromatography (0 to 60% ethyl acetate in heptane, 12 CV) to afford (6-tert-butyl-7-methyl-pyrrolo[2,3-d]pyrimidin-2-yl)methanol (1.697 g, 63%) as a white solid ¹H NMR (400 MHz, CDCl₃) δ 8.78 (s, 1H), 6.33 (s, 1H), 4.89 (d, J=4.4 Hz, 2H), 4.00 (s, 3H), 1.50 (s, 9H). ESI-MS m/z calc. 219.1372, found 220.1 (M+1)⁺; Retention time: 1.22 minutes. LC method I.

### Step 6: 6-tert-Butyl-7-methyl-pyrrolo[2,3-d]pyrimidine-2-carbaldehyde

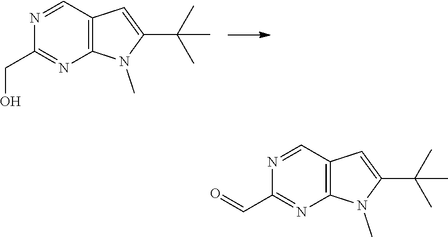 [SMILES: Cn1c(C(C)(C)C)cc2cnc(CO)nc21 ; Cn1c(C(C)(C)C)cc2cnc(C=O)nc21]

To a solution of (6-tert-butyl-7-methyl-pyrrolo[2,3-d]pyrimidin-2-yl)methanol (1.597 g, 7.2755 mmol) in DCM (40 mL) at 0° C. was added Dess-Martin periodinane (4.63 g, 10.916 mmol). The reaction was stirred at 0° C. for 5 minutes and then stirred for 2 hours at room temperature. An aqueous solution of sodium thiosulfate (10%, 150 mL) and an aqueous saturated solution of sodium bicarbonate (150 mL) were added, and the reaction mixture was stirred for 1 hour. The layers were separated and the aqueous layer was extracted with DCM (3×100 mL). The combined organic layers were washed with an aqueous solution of NaOH (1N, 300 mL) and brine (300 mL), dried over sodium sulfate, filtered and concentrated in vacuo to afford crude 6-tert-butyl-7-methyl-pyrrolo[2,3-d]pyrimidine-2-carbaldehyde (1.57 g, 98%) ¹H NMR (400 MHz, CDCl₃) δ 10.15 (s, 1H), 9.01 (s, 1H), 6.48 (s, 1H), 4.12 (s, 3H), 1.52 (s, 9H) ESI-MS m/z calc. 217.1215, found 218.2 (M+1)⁺; Retention time: 3.01 minutes ¹H NMR (400 MHz, CDCl₃) δ 10.15 (s, 1H), 9.01 (s, 1H), 6.48 (s, 1H), 4.12 (s, 3H), 1.52 (s, 9H). LC method K.

### Step 7: 3-[[4-[(2R)-3-(1-Bicyclo[1.1.1]]pentanyl)-2-[(6-tert-butyl-7-methyl-pyrrolo[2,3-d]pyrimidin-2-yl)methylamino]propoxy]-6-(2,6-dimethylphenyl)pyrimidin-2-yl]sulfamoyl]benzoic acid

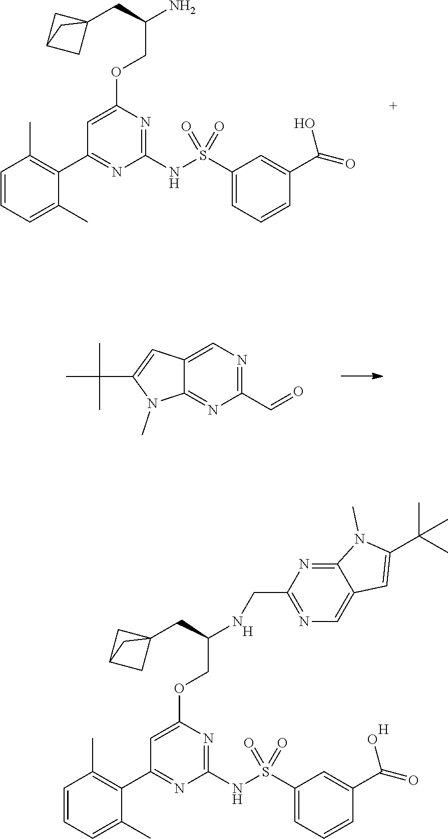

A 20 mL vial was charged under nitrogen with 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (70 mg, 0.1127 mmol), 6-tert-butyl-7-methyl-pyrrolo[2,3-d]pyrimidine-2-carbaldehyde (24.8 mg, 0.1141 mmol), anhydrous DCM (0.57 mL) and acetic acid (9.7 μL, 0.1706 mmol). The mixture was cooled down in an ice bath. DIEA (39.3 μL, 0.2256 mmol) was added, followed by sodium triacetoxyborohydride (144.0 mg, 0.6794 mmol) and the reaction was vigorously stirred at 0° C. for 4 h. The reaction was quenched with 3 N aqueous HCl, diluted with MeOH and DMSO and the resulting solution was filtered. Purification by reverse phase HPLC (1-99% acetonitrile/5 mM aqueous HCl over 15 min) provided 3-[[4-[(2R)-3-(1-bicyclo[1.1.1]pentanyl)-2-[(6-tert-butyl-7-methyl-pyrrolo[2,3-d]pyrimidin-2-yl)methylamino]propoxy]-6-(2,6-dimethylphenyl)pyrimidin-2-yl]sulfamoyl]benzoic acid (hydrochloride salt) (55.2 mg, 64%) as a yellow-brown solid. ESI-MS m/z calc. 723.3203, found 724.2 (M+1)⁺; Retention time: 1.45 minutes. LC method A.

### Step 8: (11R)-11-(1-Bicyclo[1.1.1]pentanylmethyl)-12-[(6-tert-butyl-7-methyl-pyrrolo[2,3-d]pyrimidin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-35

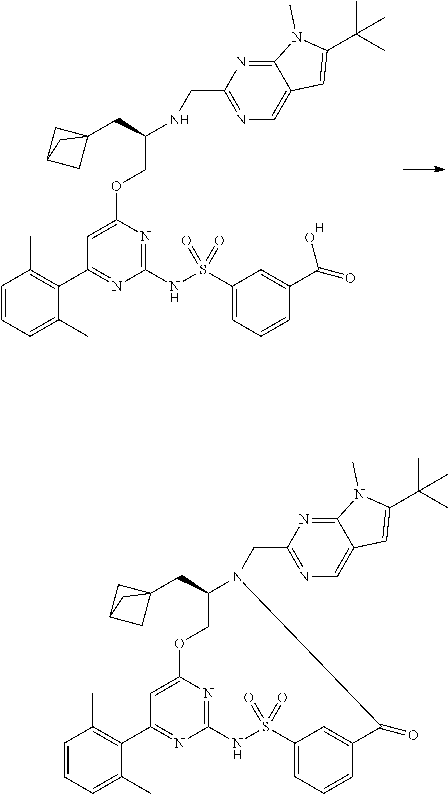

3-[[4-[(2R)-3-(1-bicyclo[1.1.1]pentanyl)-2-[(6-tert-butyl-7-methyl-pyrrolo[2,3-d]pyrimidin-2-yl)methylamino]propoxy]-6-(2,6-dimethylphenyl)pyrimidin-2-yl]sulfamoyl]benzoic acid (hydrochloride salt) (55.2 mg, 0.07260 mmol) was combined with CDMT (19.8 mg, 0.1128 mmol) in DMF (7.3 mL) and cooled to 0° C. N-methylmorpholine (24.0 μL, 0.2183 mmol) was added by syringe and the reaction was stirred at 0° C. for 30 minutes. The ice bath was then removed and stirring was continued for an additional 16 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-11-(1-bicyclo[1.1.1]pentanylmethyl)-12-[(6-tert-butyl-7-methyl-pyrrolo[2,3-d]pyrimidin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (13.9 mg, 27%) as a yellow solid. ¹H NMR (400 MHz, CDCl₃) δ 9.11 (t, J=1.8 Hz, 1H), 8.90 (s, 1H), 8.03 (d, J=7.9 Hz, 1H), 7.91 (dt, J=7.6, 1.4 Hz, 1H), 7.69 (t, J=7.8 Hz, 1H), 7.23 (t, J=7.6 Hz, 1H), 7.06 (d, J=7.6 Hz, 2H), 6.66 (s, 1H), 6.18 (s, 1H), 5.81 (d, J=17.9 Hz, 1H), 5.61 (dd, J=10.8, 3.7 Hz, 1H), 4.90 (d, J=17.9 Hz, 1H), 4.16 (s, 3H), 4.14-4.04 (m, 1H), 3.82 (t, J=11.2 Hz, 1H), 2.45 (s, 1H), 2.10-1.98 (m, 7H), 1.84 (dd, J=15.6, 3.3 Hz, 1H), 1.62 (s, 6H), 1.50 (s, 9H). ESI-MS m/z calc. 705.30975, found 706.2 (M+1)⁺; Retention time: 1.51 minutes. LC method A.

### Example 112: Preparation of Compound IV-40

### Step 1: 3-[[4-[(2R)-2-[(6-tert-Butylfuro[2,3-b]pyrazin-2-yl)methylamino]-4,4-dimethyl-pentoxy]-6-[2-(isopropoxymethyl)-6-methyl-phenyl]-5-methyl-pyrimidin-2-yl]sulfamoyl]benzoic acid

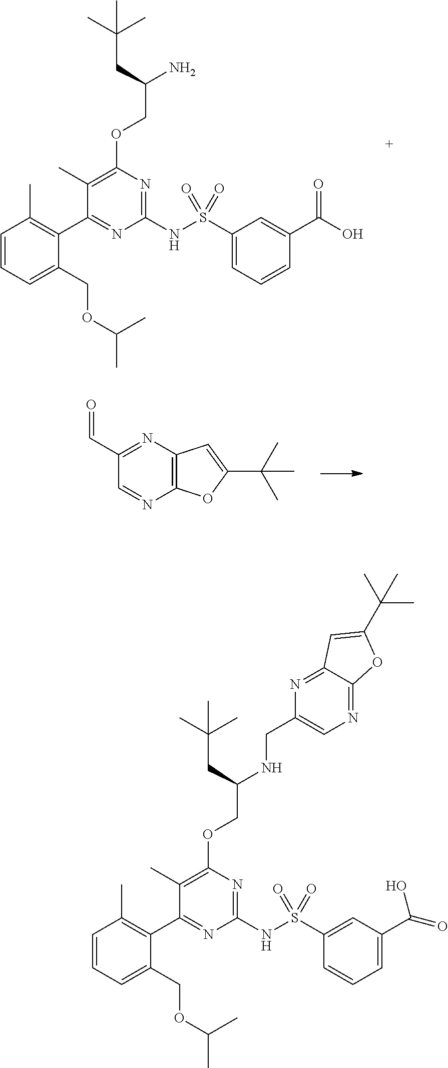

3-[[4-[(2R)-2-amino-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (50 mg, 0.08049 mmol) was combined with 6-tert-butylfuro[2,3-b]pyrazine-2-carbaldehyde (18.5 mg, 0.09059 mmol) in DCM (0.5 mL). AcOH (10 μL, 0.1758 mmol) was added and after stirring for 10 minutes at room temperature the reaction mixture was cooled to 0° C. in an ice water bath. DIPEA (50 μL, 0.2871 mmol) was added followed 20 minutes later by sodium triacetoxyborohydride (110 mg, 0.5190 mmol). Stirring was continued at 0° C. for 90 minutes. The reaction mixture was then quenched with 0.3 mL 3M HCl (aqueous) then diluted with methanol and DMSO until the reaction mixture became homogeneous. It was then filtered and purified by prep HPLC (1-99% ACN in water, HCl modifier, 15 minute run) to give 3-[[4-[(2R)-2-[(6-tert-butylfuro[2,3-b]pyrazin-2-yl)methylamino]-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (36.7 mg, 56%). ESI-MS m/z calc. 772.3618, found 773.8 (M+1)⁺; Retention time: 0.64 minutes; LC method B.

### Step 2: (5P,1R)-12-({6-tert-butylfuro[2,3-b]pyrazin-2-yl}methyl)-11-(2,2-dimethylpropyl)-7-methyl-6-{2-methyl-6-[(propan-2-yloxy)methyl]phenyl}-9-oxa-2λ⁶-thia-3,5,12,19-tetraazatricyclo[12.3.1.]∧{4,8}]nonadeca-1(18),4(19),5,7,14,16-hexaene-2,2,13-trione (Compound IV-40

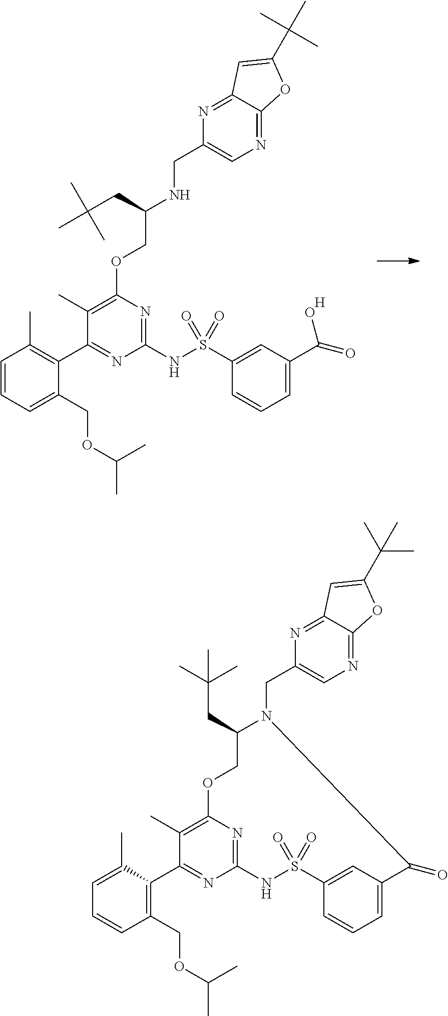

3-[[4-[(2R)-2-[(6-tert-Butylfuro[2,3-b]pyrazin-2-yl)methylamino]-4,4-dimethyl-pentoxy]-6-[2-(isopropoxymethyl)-6-methyl-phenyl]-5-methyl-pyrimidin-2-yl]sulfamoyl]benzoic acid (hydrochloride salt) (36.7 mg, 0.04534 mmol) was combined with CDMT (10.5 mg, 0.05981 mmol) in DMF (3 mL) and N-methylmorpholine (30 μL, 0.2729 mmol) was added. The reaction was stirred at room temperature for 60 hours. At this time volatiles were removed by rotary evaporation. The resulting residue was dissolved in methanol, filtered, and purified by preparative HPLC (1-99% ACN in water, HCl modifier) to give (5P,11R)-12-({6-tert-butylfuro[2,3-b]pyrazin-2-yl}methyl)-11-(2,2-dimethylpropyl)-7-methyl-6-{2-methyl-6-[(propan-2-yloxy)methyl]phenyl}-9-oxa-2λ⁶-thia-3,5,12,19-tetraazatricyclo[12.3.1.1∧{4,8}]nonadeca-1(18),4(19),5,7,14,16-hexaene-2,2,13-trione (8.1 mg, 23%) as the second isomer (peak 2) to elute. ESI-MS m/z calc. 754.35126, found 755.8 (M+1)⁺; Retention time: 2.24 minutes; LC method A. ¹H NMR (400 MHz, CDCl₃) δ 8.74 (s, 1H), 8.42 (s, 1H), 8.14 (d, J=7.8 Hz, 1H), 7.87 (d, J=7.5 Hz, 1H), 7.67 (t, J=7.8 Hz, 1H), 7.39-7.31 (m, 2H), 7.19 (d, J=6.6 Hz, 1H), 6.64 (s, 1H), 5.50 (dd, J=11.4, 4.3 Hz, 1H), 5.26 (d, J=14.7 Hz, 1H), 4.49 (t, J=11.5 Hz, 1H), 4.39-4.30 (m, 1H), 4.26 (d, J=4.6 Hz, 1H), 4.24-4.17 (m, 1H), 3.51 (p, J=6.1 Hz, 1H), 2.62 (s, 1H), 1.90 (s, 3H), 1.85-1.83 (m, 1H), 1.81 (s, 3H), 1.54 (d, J=14.1 Hz, 1H), 1.43 (s, 9H), 1.12 (d, J=6.1 Hz, 3H), 1.06 (d, J=6.0 Hz, 3H), 0.62 (s, 9H).

### Example 113: Preparation of Compound IV-42

### Step 1: 3,5-Dichloro-2-methylsulfanyl-pyrazine

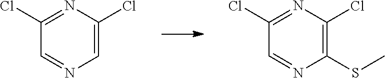

To a solution of 2,2,6,6-tetramethylpiperidine (4.1850 g, 5.0 mL, 29.627 mmol) in THE (60 mL) was added dropwise n-BuLi in hexanes (11.8 mL of 2.5 M, 29.500 mmol) at −20° C. under nitrogen. The solution was stirred for 30 min at 0° C. To the solution of LTMP prepared previously, was added dropwise 2,6-dichloropyrazine (3.75 g, 24.668 mmol) at −78° C. in THE (45 mL). After 30 min of stirring at −78° C., dimethyldisulfide (6.7990 g, 6.5 mL, 72.177 mmol) was added in THE (30 mL) at −78° C. After the mixture was stirred for 1 h at −78° C., the hydrolysis was performed with water (20 mL) at −78° C. The aqueous layer was then extracted twice with AcOEt (2×20 mL). After drying over sodium sulfate, filtration and solvent evaporation, the residue was purified by reverse phase chromatography on a 275 g C₁₈ cartridge with a gradient of MeCN in acidic water (contains 0.1% w/w formic acid) (5% for 3 CV then to 100% over 15 CV) to give 3,5-dichloro-2-methylsulfanyl-pyrazine (1.44 g, 17%) as a colorless oil. ¹H NMR (400 MHz, CDCl₃) δ 8.36 (s, 1H), 2.56 (s, 3H). ESI-MS m/z calc. 193.9472, found no ionisation; Retention time: 1.92 minutes. LC method I.

### Step 2: 5-Chloro-3-(2-cyclopropylethynyl)-2-methylsulfanyl-pyrazine

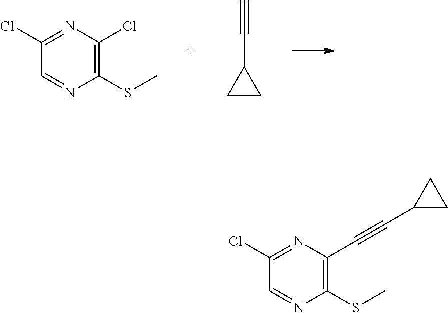

To a solution of 3,5-dichloro-2-methylsulfanyl-pyrazine (1.44 g, 5.2486 mmol) in degassed triethylamine (14 mL) was added CuI (50 mg, 0.2625 mmol) and bis(triphenylphosphine)palladium(II)chloride (185 mg, 0.2636 mmol). The mixture was purged with nitrogen for 5 minutes and then ethynylcyclopropane (421.74 mg, 0.54 mL, 6.3802 mmol) was added. The mixture was stirred for 1.5 hours at 90° C. The temperature was decreased to RT then a new amount of ethynylcyclopropane (101.53 mg, 0.13 mL, 1.5360 mmol) was added. The mixture was stirred for 1.5 hours at 90° C. The mixture was diluted with EtOAc (100 mL), then the solution was filtered on a pad of Celite and the pad was rinsed with EtOAc (200 mL). The filtrate was washed with water (3×100 mL) and brine (1×100 mL), dried over sodium sulfate, filtered and concentrated in vacuo. The residue was purified by chromatography on a cartridge of silica gel, eluting from 0% to 20% of ethyl acetate in heptanes to give 5-chloro-3-(2-cyclopropylethynyl)-2-methylsulfanyl-pyrazine (350 mg, 27%) as a clear oil. ¹H NMR (400 MHz, CDCl₃) δ 8.26 (s, 1H), 2.52 (s, 3H), 1.61-1.56 (m, 1H), 1.03-0.96 (m, 4H). ESI-MS m/z calc. 224.0175, found 225.2 (M+1)⁺; Retention time: 2.0 minutes. LC method I.

### Step 3: 2-Chloro-6-cyclopropyl-7-iodo-thieno[2,3-b]pyrazine

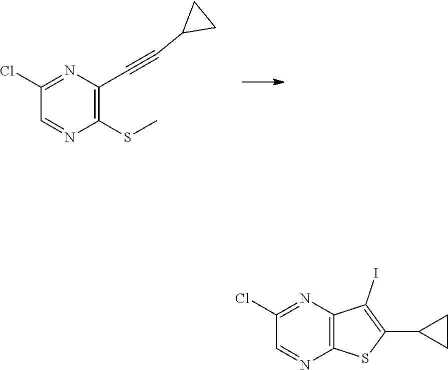

To a solution of 5-chloro-3-(2-cyclopropylethynyl)-2-methylsulfanyl-pyrazine (350 mg, 1.5576 mmol) in DCM (70 mL) was added iodine (791 mg, 3.1165 mmol). The reaction was stirred at RT overnight. The reaction mixture was quenched with saturated sodium thiosulfate (20 mL) and was extracted with DCM (45 mL). The organic phase was concentrated under reduced pressure to give 2-chloro-6-cyclopropyl-7-iodo-thieno[2,3-b]pyrazine (496 mg, 83%) as a crude yellow solid. ¹H NMR (400 MHz, CDCl₃) δ 8.37 (s, 1H), 2.53-2.46 (m, 1H), 1.38-1.32 (m, 2H), 1.06-1.01 (m, 2H). ESI-MS m/z calc. 335.8985, found 336.8 (M+1)⁺; Retention time: 2.06 minutes. LC method I.

### Step 4: 2-Chloro-6-cyclopropyl-thieno[2,3-b]pyrazine

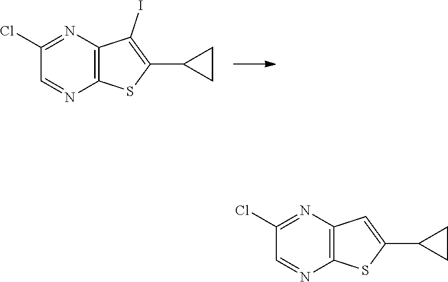 [SMILES: Clc1cnc2sc(C3CC3)c(I)c2n1 ; Clc1cnc2sc(C3CC3)cc2n1]

Pd (PPh₃)₂Cl₂ (52 mg, 0.0741 mmol) and triethylamine (450.12 mg, 0.62 mL, 4.4483 mmol) were added to a solution of 2-chloro-6-cyclopropyl-7-iodo-thieno[2,3-b]pyrazine (496 mg, 1.2880 mmol) and formic acid (146.40 mg, 0.12 mL, 3.1809 mmol) in DMF (15 mL). The resulting mixture was heated at 60° C. for 12 h. The residue was diluted in diethyl ether (75 mL) then was washed with water (2×15 mL), brine (15 mL), dried over sodium sulfate, filtered, and concentrated under high vacuum. The residue was purified by chromatography on a silica gel cartridge, eluting from 0% to 20% of ethyl acetate in heptanes to give 2-chloro-6-cyclopropyl-thieno[2,3-b]pyrazine (171 mg, 44%) as a beige solid. ¹H NMR (400 MHz, CDCl₃) δ 8.36 (s, 1H), 7.08 (d, J=0.7 Hz, 1H), 2.33-2.23 (m, 1H), 1.28-1.21 (m, 2H), 1.01-0.95 (m, 2H). ESI-MS m/z calc. 210.0018, found 211.2 (M+1)⁺; Retention time: 1.92 minutes. LC method I.

### Step 5: Methyl 6-cyclopropylthieno[2,3-b]pyrazine-2-carboxylate

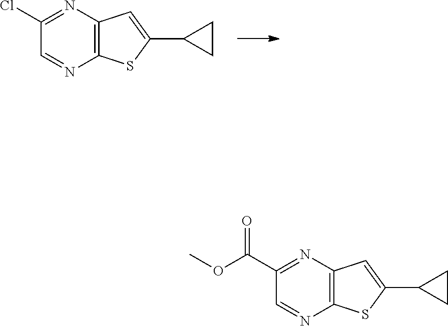 [SMILES: Clc1cnc2sc(C3CC3)cc2n1 ; COC(=O)c1cnc2sc(C3CC3)cc2n1]

In a seal tube, a solution of 2-chloro-6-cyclopropyl-thieno[2,3-b]pyrazine (171 mg, 0.8116 mmol) in methanol (4 mL) was mixed with triethylamine (166.98 mg, 0.23 mL, 1.6502 mmol). The system was purged with nitrogen then added 1,1′-bis(diphenylphosphino)-ferrocene palladium(II) chloride, complex with dichloromethane (34 mg, 0.0416 mmol). The reaction was stirred at 100° C. under carbone monoxide atmosphere (50 psi). The mixture was stirred overnight at 100° C., then cooled down to 25° C. The mixture was purged with nitrogen and concentrated under vacuum. The resulting solid was diluted with ethyl acetate (35 mL) and water (15 mL) The layers were separated. Extract with EtOAc (2×15 mL). The organic layers were combined then was washed with water (15 mL) and brine (15 mL), dried over sodium sulfate, filtered, and evaporated under reduced pressure. The residue was purified by chromatography on a silica gel cartridge, eluting from 10% to 40% of ethyl acetate in heptanes to give methyl 6-cyclopropylthieno[2,3-b]pyrazine-2-carboxylate (108 mg, 54%) as an off white solid. ¹H NMR (400 MHz, CDCl₃) δ 9.13 (s, 1H), 7.29-7.27 (overlapped with solvent, m, 1H), 4.08 (s, 3H), 2.36-2.26 (m, 1H), 1.30-1.23 (m, 2H), 1.03-0.97 (m, 2H). ESI-MS m/z calc. 234.0463, found 235.2 (M+1)⁺; Retention time: 1.71 minutes. LC method I.

### Step 6: (6-Cyclopropylthieno[2,3-b]pyrazin-2-yl)methanol

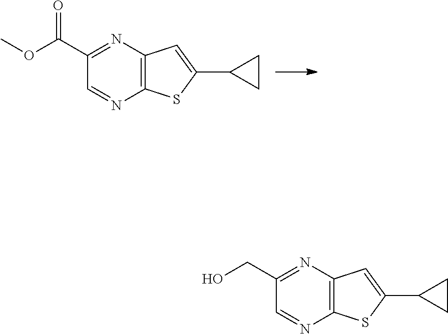 [SMILES: COC(=O)c1cnc2sc(C3CC3)cc2n1 ; OCc1cnc2sc(C3CC3)cc2n1]

To a solution of methyl 6-cyclopropylthieno[2,3-b]pyrazine-2-carboxylate (108 mg, 0.4610 mmol) in methanol (3 mL) at RT was added lithium borohydride (26 mg, 1.1935 mmol). The reaction was stirred at RT for 4 hours. The reaction was quenched carefully with water (1 mL). The reaction was diluted with water (10 mL) then extracted with EtOAc (3×15 mL). The combined organics phases were washed with brine (10 mL) then dried over sodium sulfate, filtered and concentrated under high vacuum to give (6-cyclopropylthieno[2,3-b]pyrazin-2-yl)methanol (97 mg, 97%) as a beige solid. ¹H NMR (400 MHz, CDCl₃) δ 8.40 (s, 1H), 7.12 (s, 1H), 4.92 (s, 2H), 3.17 (br. s., 1H), 2.33-2.21 (m, 1H), 1.30-1.18 (overlapped with solvent, m, 2H), 1.02-0.93 (m, 2H). ESI-MS m/z calc. 206.0514, found 207.2 (M+1)⁺; Retention time: 1.48 minutes. LC method I.

### Step 7: 6-Cyclopropylthieno[2,3-b]pyrazine-2-carbaldehyde

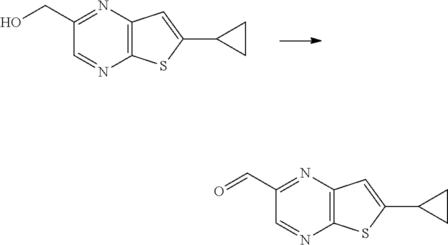 [SMILES: OCc1cnc2sc(C3CC3)cc2n1 ; O=Cc1cnc2sc(C3CC3)cc2n1]

To a solution of (6-cyclopropylthieno[2,3-b]pyrazin-2-yl)methanol (97 mg, 0.4703 mmol) in DCM (3 mL) at 0° C. was added Dess-martin periodinane (300 mg, 0.7073 mmol). The reaction was stirred at RT for two hours. The reaction was quenched with a mixture 1:1 (4 mL) of aqueous solution of sodium thiosulfate 10% and aqueous saturated sodium bicarbonate. The solution was stirred 15 minutes then diluted in EtOAc (50 mL). The organic phase was separated then washed with water (10 mL), brine (10 mL), dried over sodium sulfate, filtered, and concentrated under reduced pressure. The residue was purified by chromatography on silica gel cartridge, eluting from 0% to 30% of ethyl acetate in heptanes to give 6-cyclopropylthieno[2,3-b]pyrazine-2-carbaldehyde (60 mg, 61%) as a white solid. ¹H NMR (400 MHz, CDCl₃) δ 10.20 (s, 1H), 8.98 (s, 1H), 7.26-7.23 (m, 1H), 2.39-2.26 (m, 1H), 1.35-1.23 (m, 2H), 1.10-0.96 (m, 2H). ESI-MS m/z calc. 204.0357, found 205.1 (M+1)⁺; Retention time: 2.58 minutes. LC method F.

### Step 8: 3-[[4-[(2R)-3-(1-Bicyclo[1.1.1]]pentanyl)-2-[(6-cyclopropylthieno[2,3-b]pyrazin-2-yl)methylamino]propoxy]-6-(2,6-dimethylphenyl)pyrimidin-2-yl]sulfamoyl]benzoic acid

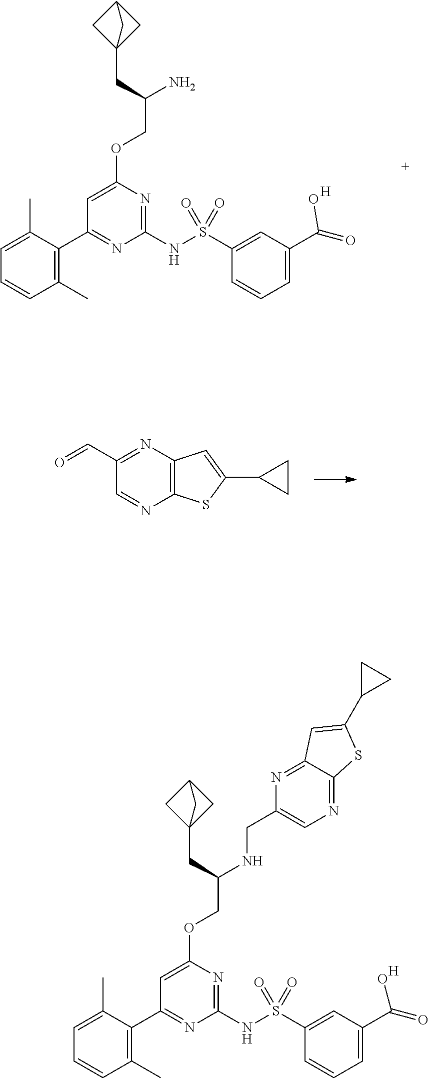

3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (25 mg, 0.04024 mmol) was combined with 6-cyclopropylthieno[2,3-b]pyrazine-2-carbaldehyde (9.1 mg, 0.04455 mmol) in DCM and AcOH (6 μL, 0.1055 mmol). After stirring for 15 minutes at room temperature the reaction mixture was cooled to 0° C. in an ice bath. DIPEA (20 μL, 0.1148 mmol) was then added and after 15 minutes sodium triacetoxyborohydride (approximately 50.93 mg, 0.2403 mmol) was added, and the reaction was stirred for 90 minutes at 0° C. The reaction mixture was then quenched with 0.3 mL 3M HCl then diluted with DMSO and methanol until the reaction mixture became homogeneous. The reaction mixture was then filtered and purified by preparative HPLC (1-99% ACN in water, HCl modifier, 15 minute run) to give 3-[[4-[(2R)-3-(1-bicyclo[1.1.1]pentanyl)-2-[(6-cyclopropylthieno[2,3-b]pyrazin-2-yl)methylamino]propoxy]-6-(2,6-dimethylphenyl)pyrimidin-2-yl]sulfamoyl]benzoic acid (hydrochloride salt) (20 mg, 61%). ESI-MS m/z calc. 710.2345, found 711.8 (M+1)⁺; Retention time: 0.59 minutes; LC method B.

### Step 9: (11R)-11-(1-Bicyclo[0.1.]pentanylmethyl)-12-[(6-cyclopropylthieno[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-42

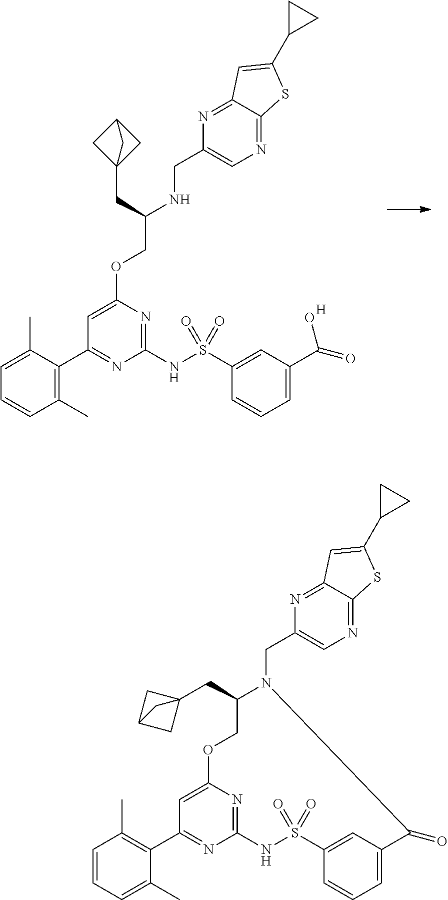

3-[[4-[(2R)-3-(1-Bicyclo[1.1.1]pentanyl)-2-[(6-cyclopropylthieno[2,3-b]pyrazin-2-yl)methylamino]propoxy]-6-(2,6-dimethylphenyl)pyrimidin-2-yl]sulfamoyl]benzoic acid (hydrochloride salt) (20 mg, 0.02676 mmol) was combined with CDMT (6 mg, 0.03417 mmol) in anhydrous DMF (3 mL). N-methylmorpholine (20 μL, 0.1819 mmol) was added dropwise and the reaction was stirred at room temperature for 18 hours. The reaction mixture was filtered and purified by preparative HPLC (1-99% ACN in water, HCl modifier, 15 minute run) to give (11R)-11-(1-bicyclo[1.1.1]pentanylmethyl)-12-[(6-cyclopropylthieno[2,3-b]pyrazin-2-yl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (10.2 mg, 54%). ESI-MS m/z calc. 692.22394, found 693.7 (M+1)⁺; Retention time: 2.06 minutes; LC method A. ¹H NMR (400 MHz, CDCl₃) δ 8.84 (s, 1H), 8.58 (s, 1H), 8.12 (d, J=7.9 Hz, 1H), 7.90 (d, J=7.5 Hz, 1H), 7.67 (t, J=7.8 Hz, 1H), 7.21 (t, J=7.6 Hz, 1H), 7.15 (d, J=0.8 Hz, 1H), 7.06 (d, J=7.6 Hz, 2H), 6.23 (s, 1H), 5.45 (dd, J=10.8, 3.4 Hz, 1H), 5.34 (d, J=15.3 Hz, 1H), 4.24 (d, J=15.3 Hz, 1H), 4.13 (t, J=11.2 Hz, 1H), 4.10-3.99 (m, 1H), 2.44 (s, 1H), 2.24 (ddd, J=13.3, 8.4, 4.8 Hz, 1H), 2.04 (s, 6H), 2.00-1.94 (m, 1H), 1.82 (dd, J=15.7, 3.1 Hz, 1H), 1.60-1.55 (m, 6H), 1.25-1.13 (m, 2H), 1.04-0.92 (m, 2H).

### Example 114: Preparation of Compound IV-48

### Step 1: (2R)-2-Amino-3-ethyl-pentanoic acid

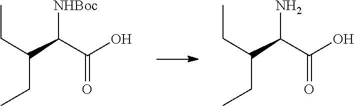

(2R)-2-(tert-Butoxycarbonylamino)-3-ethyl-pentanoic acid (250 mg, 1.019 mmol) was dissolved in HCl (approximately 2.548 mL of 4 M, 10.19 mmol) (in dioxane) and stirred at room temperature for 90 minutes. The reaction mixture was then diluted with DCM and hexanes and the volatiles were evaporated. DCM and hexanes were added a second time and this process was repeated to give as a white solid, (2R)-2-amino-3-ethyl-pentanoic acid (hydrochloride salt) (189 mg, 92%) ESI-MS m/z calc. 145.11028, found 146.1 (M+1)⁺; Retention time: 0.2 minutes; LC method B.

### Step 2: (2R)-2-Amino-3-ethyl-pentan-1-ol

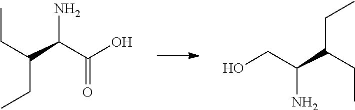

To a solution of (2R)-2-amino-3-ethyl-pentanoic acid (hydrochloride salt) (189 mg, 0.9884 mmol) in Tetrahydrofuran (1.795 mL) was added borane tetrahydrofuran complex (4 mL of 1 M, 4.000 mmol) at 0° C. dropwise. The reaction was allowed to warm to room temperature and stirred for the indicated time. The reaction was quenched with slow addition of methanol (30 mL) and then co-evaporated with methanol (3×50 mL) and DCE (1×5 mL). The crude material was then suspended in DCM (50 mL) and acidified with HCl (1.5 mL of 4 M, 6.000 mmol) in dioxane to give (2R)-2-amino-3-ethyl-pentan-1-ol (hydrochloride salt) (163 mg, 98%) ESI-MS m/z calc. 131.13101, found 132.1 (M+1)⁺; Retention time: 0.36 minutes; Final purity was determined by reversed phase UPLC using an Acquity UPLC BEH C₁₈ column (30×2.1 mm, 1.7 m particle) made by Waters (pn: 186002349), and a dual gradient run from 1-30% mobile phase B over 0.9 minutes, with a 0.1 minute ramp to 99%, 0.1 minute hold at 99%, and 0.1 minute ramp to 1%. Mobile phase A=H₂O (0.05% TFA). Mobile phase B=acetonitrile(0.035% TFA). Flow rate=1.5 mL/min, injection volume=2 μL, and column temperature=60° C.

### Step 3: 3-[[4-[(2R)-2-Amino-3-ethyl-pentoxy]-6-(2,6-dimethylphenyl)pyrimidin-2-yl]sulfamoyl]benzoic acid

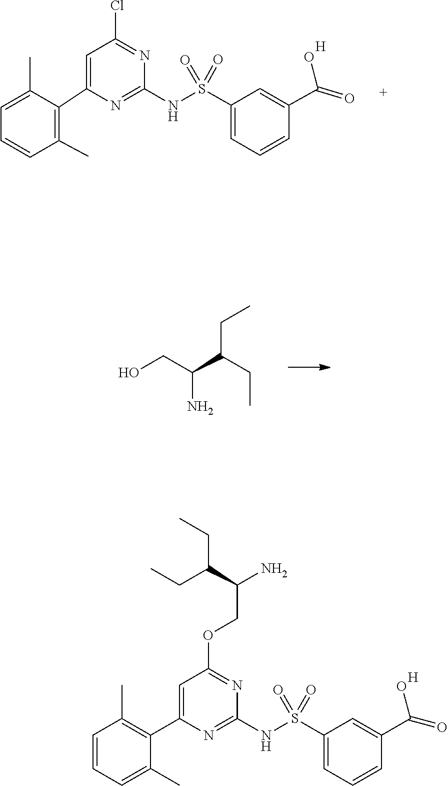

3-[[4-Chloro-6-(2,6-dimethylphenyl)pyrimidin-2-yl]sulfamoyl]benzoic acid (300 mg, 0.7179 mmol) was combined with (2R)-2-amino-3-ethyl-pentan-1-ol (hydrochloride salt) (132 mg, 0.7872 mmol) in THE (1.8 mL) and stirred for 5 minutes. Sodium tert-butoxide (350 mg, 3.642 mmol) was then added in one portion, and the reaction mixture was stirred vigorously at room temperature for 30 minutes. The temperature was increased to 45° C. for 30 minutes, then the reaction was cooled to room temperature and partitioned between 1 M HCl and ethyl acetate. The layers were separated and the aqueous was extracted an additional 2× with ethyl acetate. The aqueous layer was then diluted with brine and extracted a further 2× ethyl acetate. The combined organics were washed with brine, dried over sodium sulfate and concentrated to give 3-[[4-[(2R)-2-amino-3-ethyl-pentoxy]-6-(2,6-dimethylphenyl)pyrimidin-2-yl]sulfamoyl]benzoic acid (hydrochloride salt) (391.9 mg, 91%), which was used without further purification. ESI-MS m/z calc. 512.20935, found 513.5 (M+1)⁺; Retention time: 0.48 minutes; LC method B.

### Step 4: 3-[[4-[(2R)-2-[(6-tert-Butylfuro[2,3-b]pyrazin-2-yl)methylamino]-3-ethyl-pentoxy]-6-(2,6-dimethylphenyl)pyrimidin-2-yl]sulfamoyl]benzoic acid

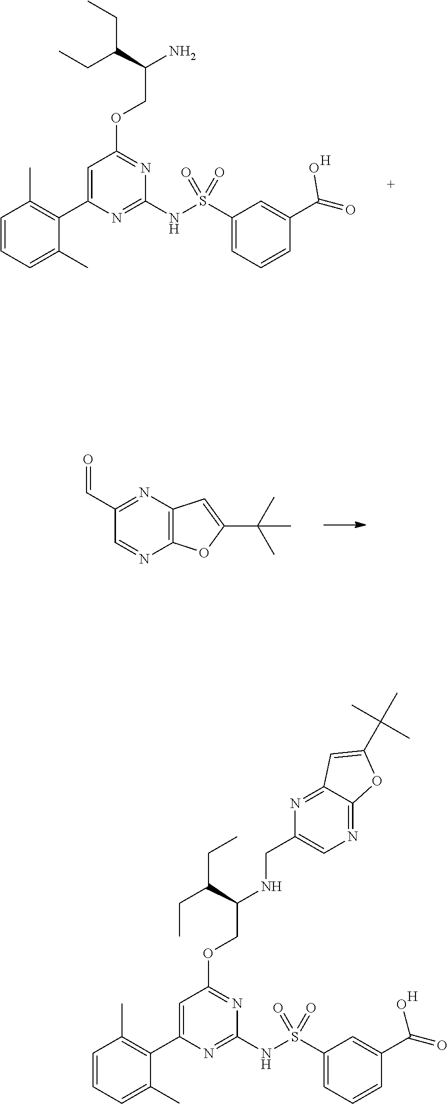

3-[[4-[(2R)-2-Amino-3-ethyl-pentoxy]-6-(2,6-dimethylphenyl)pyrimidin-2-yl]sulfamoyl]benzoic acid (hydrochloride salt) (30 mg, 0.05027 mmol) was combined with 6-tert-butylfuro[2,3-b]pyrazine-2-carbaldehyde (11.3 mg, 0.05533 mmol) in DCM and AcOH (6 μL, 0.1055 mmol). After stirring for 15 minutes at room temperature the reaction mixture was cooled to 0° C. in an ice bath. DIPEA (25 μL, 0.1435 mmol) was then added and after 15 minutes sodium triacetoxyborohydride (70 mg, 0.3303 mmol) was added, and the reaction was stirred for 90 minutes at 0° C. The reaction mixture was then quenched with 0.3 mL 3M HCl then diluted with DMSO and methanol until the reaction mixture became homogeneous. The reaction mixture was then filtered and purified by preparative HPLC (1-99% ACN in water, HCl modifier, 15 minute run) to give 3-[[4-[(2R)-2-[(6-tert-butylfuro[2,3-b]pyrazin-2-yl)methylamino]-3-ethyl-pentoxy]-6-(2,6-dimethylphenyl)pyrimidin-2-yl]sulfamoyl]benzoic acid (hydrochloride salt) (24.6 mg, 66%). ESI-MS m/z calc. 700.3043, found 701.8 (M+1)⁺; Retention time: 0.61 minutes; LC method B.

### Step 5: (11R)-12-[(6-tert-Butylfuro[2,3-b]pyrazin-2-yl)methyl]-6-(2,6-dimethylphenyl)-11-(1-ethylpropyl)-2,2-dioxo-9-oxa-2λ⁶-thia-3,5,12,19-tetrazatricyclo[12.3.1.14,8]-nonadeca-1(18),4,6,8(19),14,16-hexaen-13-one (Compound IV-48

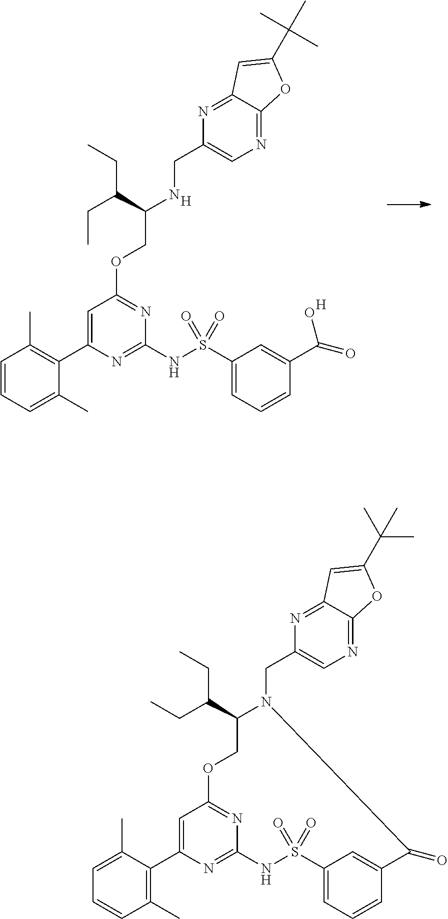

3-[[4-[(2R)-2-[(6-tert-Butylfuro[2,3-b]pyrazin-2-yl)methylamino]-3-ethyl-pentoxy]-6-(2,6-dimethylphenyl)pyrimidin-2-yl]sulfamoyl]benzoic acid (hydrochloride salt) (24.6 mg, 0.03336 mmol) was combined with CDMT (7.5 mg, 0.04272 mmol) in anhydrous DMF (3 mL). N-methylmorpholine (25 μL, 0.2274 mmol) was added dropwise and the reaction was stirred at room temperature for 18 hours. The reaction mixture was filtered and purified by preparative HPLC (1-99% ACN in water, HCl modifier, 15 minute run) to give (11R)-12-[(6-tert-butylfuro[2,3-b]pyrazin-2-yl)methyl]-6-(2,6-dimethylphenyl)-11-(1-ethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one (12.2 mg, 52%). ESI-MS m/z calc. 682.29376, found 683.7 (M+1)⁺; Retention time: 2.14 minutes; LC method A. ¹H NMR (400 MHz, CDCl₃) δ 8.76 (t, J=1.9 Hz, 1H), 8.37 (s, 1H), 8.08 (dt, J=8.2, 1.3 Hz, 1H), 7.84 (dt, J=7.8, 1.3 Hz, 1H), 7.64 (t, J=7.8 Hz, 1H), 7.21 (t, J=7.6 Hz, 1H), 7.07 (d, J=7.6 Hz, 2H), 6.64 (s, 1H), 6.24 (s, 1H), 5.52 (dd, J=12.0, 4.2 Hz, 1H), 5.37 (d, J=14.9 Hz, 1H), 4.63 (t, J=11.8 Hz, 1H), 4.25 (d, J=14.9 Hz, 1H), 4.17 (ddd, J=11.6, 7.3, 4.2 Hz, 1H), 2.04 (s, 6H), 1.72-1.68 (m, 1H), 1.51-1.44 (m, 3H), 1.42 (s, 9H), 1.23-1.16 (m, 1H), 0.77 (t, J=7.3 Hz, 3H), 0.48 (t, J=7.4 Hz, 3H).

### Example 115: Preparation of Compound IV-49

### Step 1: 2-Isopropylpyrazolo[1,5-a]pyrimidine-5,7-diol

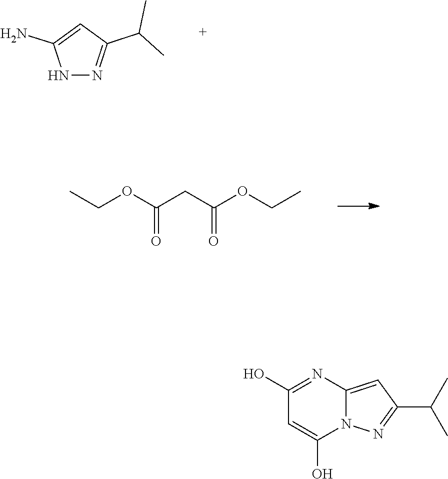

Sodium (1.47 g, 63.941 mmol) was added in ethanol (128 mL). The solution was stirred at RT until complete dissolution. Diethyl malonate (2.9540 g, 2.8 mL, 18.443 mmol) was added to the solution of sodium ethoxide then 3-isopropyl-1H-pyrazol-5-amine (2 g, 11.984 mmol) and this solution was heated at reflux for overnight. After cooling at RT, an aqueous solution of HCl (5N, 12 mL) was added until pH=5 was obtained. Volatiles were then removed under reduce pressure and the residue was dried under high vacuum to afford 2-isopropylpyrazolo[1,5-a]pyrimidine-5,7-diol as a beige solid. ESI-MS m/z calc. 193.0851, found 194.2 (M+1)⁺; Retention time: 1.15 minutes. LC method I.

### Step 2: 5,7-Dichloro-2-isopropyl-pyrazolo[1,5-a]pyrimidine

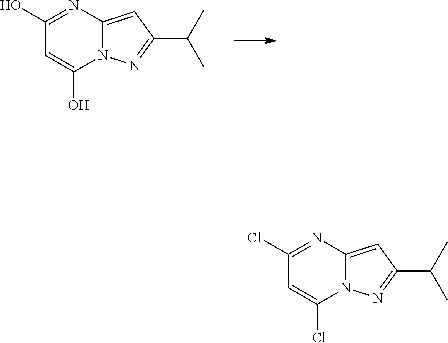

To the crude 2-isopropylpyrazolo[1,5-a]pyrimidine-5,7-diol (2.32 g, 12.008 mmol) was added POCl₃ (59.220 g, 36 mL, 386.22 mmol) and N,N-dimethylaniline (3.9196 g, 4.1 mL, 32.345 mmol) and the resulting mixture was refluxed during 3 hours. After cooling to room temperature, the reaction mixture was poured on ice and stirred one hour at this temperature. The reaction mixture was extracted with DCM (3×100 mL) and the resulting organic phase was washed with water (2×75 mL). The organic phase was dried over sodium sulfate, filtered and concentrated to give 5,7-dichloro-2-isopropyl-pyrazolo[1,5-a]pyrimidine (2.32 g, 72%) as a beige solid. ¹H NMR (400 MHz, DMSO-d6) δ 7.55 (s, 1H), 6.75 (s, 1H), 3.22-3.09 (m, 1H), 1.31 (d, J=6.8 Hz, 6H). ESI-MS m/z calc. 229.0174, found 230.2 (M+1)⁺; Retention time: 1.9 minutes. LC method I.

### Step 3: 5-Chloro-2-isopropyl-pyrazolo[1,5-a]pyrimidine

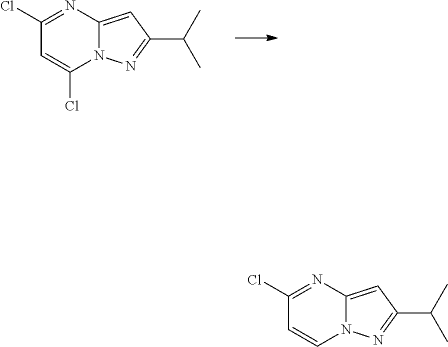

To a solution of 5,7-dichloro-2-isopropyl-pyrazolo[1,5-a]pyrimidine (2.97 g, 12.908 mmol) in acetic acid (62 mL) was added zinc dust (3.38 g, 0.4739 mL, 51.690 mmol). The mixture was stirred at rt for 6 days. The solvent was evaporated under reduced pressure and the reaction mixture was poured on water (150 mL), quenched with sodium bicarbonate solid to pH=8 then the product was extracted with DCM (3×75 ml). The combined organic layer were dried over sodium sulfate, filtered and concentrated under high vacuum. The residue was purified by chromatography on a silica gel cartridge, eluting from 5% to 30% of ethyl acetate in heptanes to give 5-chloro-2-isopropyl-pyrazolo[1,5-a]pyrimidine (331 mg, 13%) as a beige solid. ¹H NMR (400 MHz, CDCl₃) δ 8.53-8.43 (m, 1H), 6.71 (d, J=7.1 Hz, 1H), 6.42 (s, 1H), 3.16 (spt, J=7.0 Hz, 1H), 1.36 (d, J=7.1 Hz, 6H). ¹³C NMR (101 MHz, CDCl₃) δ 166.7, 149.8, 147.9, 135.8, 107.8, 93.7, 28.7, 22.5. ESI-MS m/z calc. 195.0563, found 196.2 (M+1)⁺; Retention time: 1.75 minutes, LC method I.

### Step 4: Methyl 2-isopropylpyrazolo[1,5-a]pyrimidine-5-carboxylate

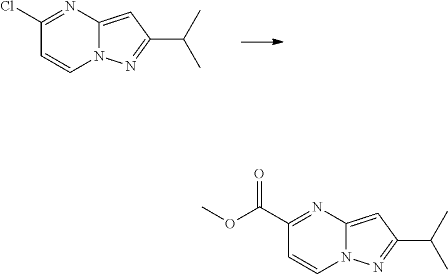 [SMILES: CC(C)c1cc2nc(Cl)ccn2n1 ; COC(=O)c1ccn2nc(C(C)C)cc2n1]

In a seal tube, a solution of 5-chloro-2-isopropyl-pyrazolo[1,5-a]pyrimidine (745 mg, 3.8078 mmol) in methanol (15 mL) was mixed with triethylamine (798.60 mg, 1.1 mL, 7.8921 mmol). The system was purged with nitrogen then 1,1′-bis(diphenylphosphino)ferrocene palladium(II) chloride, complex with dichloromethane (156 mg, 0.1910 mmol) was added. The reaction was stirred at 100° C. under carbon monoxide atmosphere (50 psi). The mixture was stirred overnight at 100° C., then cooled down to 25° C. The mixture was purged with nitrogen and concentrated under vacuum. The resulting solid was diluted with ethyl acetate (100 mL) and water (40 mL) The layers were separated, and the product was extracted with EtOAc (2×40 mL). The organic layers were combined and were washed with water (50 mL) and brine (50 mL), dried over sodium sulfate, filtered, and evaporated under reduced pressure. The residue was purified by chromatography on silica gel cartridge, eluting from 0% to 60% of ethyl acetate in heptanes to give methyl 2-isopropylpyrazolo[1,5-a]pyrimidine-5-carboxylate (613 mg, 73%) as an off-white solid. ¹H NMR (400 MHz, CDCl₃) δ 8.72 (dd, J=7.2, 0.9 Hz, 1H), 7.51 (d, J=7.1 Hz, 1H), 6.75 (s, 1H), 4.06 (s, 3H), 3.23 (spt, J=6.9 Hz, 1H), 1.40 (d, J=6.8 Hz, 6H). ESI-MS m/z calc. 219.1008, found 220.2 (M+1)⁺; Retention time: 1.59 minutes, LC method I.

### Step 5: (2-Isopropylpyrazolo[1,5-a]pyrimidin-5-yl)methanol

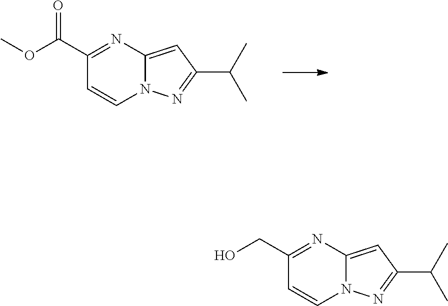 [SMILES: COC(=O)c1ccn2nc(C(C)C)cc2n1 ; CC(C)c1cc2nc(CO)ccn2n1]

To a solution of methyl 2-isopropylpyrazolo[1,5-a]pyrimidine-5-carboxylate (613 mg, 2.7960 mmol) in methanol (15 mL) at RT was added lithium borohydride (160 mg, 7.3449 mmol). The reaction was stirred at RT for 2.5 hours. The reaction was quenched carefully with water (10 mL). The reaction was diluted with water (50 mL) then was extracted with EtOAc (3×40 mL). The combined organics phases were washed with brine (30 mL) then dried over sodium sulfate, filtered, and concentrated under high vacuum. The residue was purified by chromatography on silica gel cartridge, eluting from 50% to 100% of ethyl acetate in heptanes follow by 0% to 10% of methanol in ethyl acetate to give (2-isopropylpyrazolo[1,5-a]pyrimidin-5-yl)methanol (118 mg, 21%) as a beige solid. ¹H NMR (400 MHz, CDCl₃) δ 8.54 (dd, J=7.2, 0.9 Hz, 1H), 6.65 (d, J=7.1 Hz, 1H), 6.45 (s, 1H), 4.78 (d, J=4.9 Hz, 2H), 3.62 (t, J=5.0 Hz, 1H), 3.18 (spt, J=6.9 Hz, 1H), 1.39 (d, J=6.8 Hz, 6H). ESI-MS m/z calc. 191.1059, found 192.2 (M+1)⁺; Retention time: 1.38 minutes. LC method I.

### Step 6: 2-Isopropylpyrazolo[1,5-a]pyrimidine-5-carbaldehyde

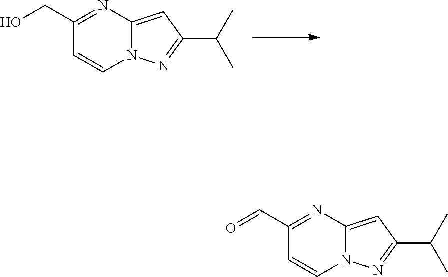

To a solution of (2-isopropylpyrazolo[1,5-a]pyrimidin-5-yl)methanol (118 mg, 0.6171 mmol) in DCM (4 mL) at 0° C. was added Dess-martin periodinane (393 mg, 0.9266 mmol). The reaction was stirred at RT for two hours. The reaction was quenched with a mixture 1:1 (4 mL) of aqueous solution of sodium thiosulfate 10% and aqueous saturated sodium bicarbonate. The solution was stirred 15 minutes then diluted in EtOAc (50 mL). The organic phase was separated then washed with water (10 mL), brine (10 mL), dried over sodium sulfate, filtered, and concentrated under reduced pressure. The residue was purified by chromatography on silica gel cartridge, eluting from 0% to 40% of ethyl acetate in heptanes. The product was dissolved in minimum of acetonitrile and water and freeze-dried overnight to provide 2-isopropylpyrazolo[1,5-a]pyrimidine-5-carbaldehyde (68 mg, 58%) as a yellow solid. ¹H NMR (400 MHz, CDCl₃) δ 9.99 (s, 1H), 8.73 (d, J=7.1 Hz, 1H), 7.36 (d, J=7.1 Hz, 1H), 6.79 (s, 1H), 3.32-3.17 (m, 1H), 1.42 (d, J=6.8 Hz, 6H). ESI-MS m/z calc. 189.0902, found 190.2 (M+1)⁺; Retention time: 2.89 minutes, LC method K.

### Step 7: 3-[[4-(2,6-Dimethylphenyl)-6-[(2R)-2-[(2-isopropylpyrazolo[1,5-a]pyrimidin-5-yl)methylamino]-3-(1-methylcyclopropyl)propoxy]pyrimidin-2-yl]sulfamoyl]benzoic acid

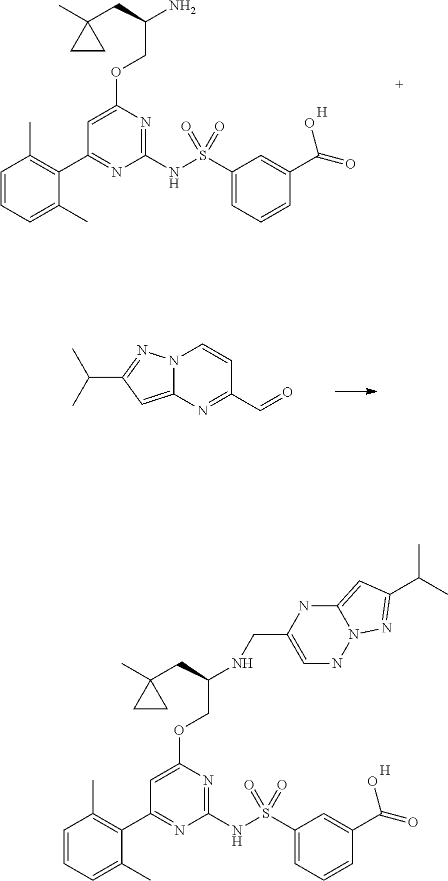

A 4 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (52.8 mg, 0.09651 mmol), 2-isopropylpyrazolo[1,5-a]pyrimidine-5-carbaldehyde (18.5 mg, 0.09777 mmol), anhydrous DCM (0.5 mL) and acetic acid (8.3 μL, 0.1460 mmol). The mixture was cooled down in an ice bath. DIEA (33.7 μL, 0.1935 mmol) was added, followed by sodium triacetoxyborohydride (103 mg, 0.4860 mmol) and the reaction was vigorously stirred at 0° C. for 2 h. The reaction was quenched with 3 N aqueous HCl, diluted with MeOH and DMSO and the resulting solution was filtered. Purification by reverse phase HPLC (1-99% acetonitrile/5 mM aqueous HCl over 15 min) provided 3-[[4-(2,6-dimethylphenyl)-6-[(2R)-2-[(2-isopropylpyrazolo[1,5-a]pyrimidin-5-yl)methylamino]-3-(1-methylcyclopropyl)propoxy]pyrimidin-2-yl]sulfamoyl]benzoic acid (hydrochloride salt) (44.9 mg, 63%) as a white solid. ESI-MS m/z calc. 683.289, found 684.2 (M+1)⁺; Retention time: 1.29 minutes. LC method A.

### Step 8: (11R)-6-(2,6-Dimethylphenyl)-12-[(2-isopropylpyrazolo[1,5-a]pyrimidin-5-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-49

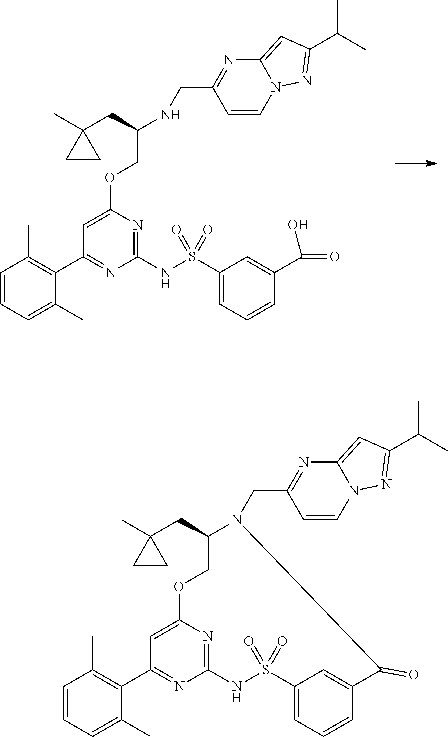 [SMILES: Cc1cccc(C)c1-c1cc(OC[C@@H](CC2(C)CC2)NCc2ccn3nc(C(C)C)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccn3nc(C(C)C)cc3n1)[C@H](CC1(C)CC1)CO2]

3-[[4-(2,6-Dimethylphenyl)-6-[(2R)-2-[(2-isopropylpyrazolo[1,5-a]pyrimidin-5-yl)methylamino]-3-(1-methylcyclopropyl)propoxy]pyrimidin-2-yl]sulfamoyl]benzoic acid (hydrochloride salt) (44.9 mg, 0.06047 mmol) was combined with CDMT (17.0 mg, 0.09683 mmol) in DMF (6.1 mL) and cooled to 0° C. N-methylmorpholine (20.0 μL, 0.1819 mmol) was added by syringe and the reaction was stirred at 0° C. for 30 minutes. The ice bath was then removed and stirring was continued for an additional 16 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-6-(2,6-dimethylphenyl)-12-[(2-isopropylpyrazolo[1,5-a]pyrimidin-5-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (18.9 mg, 43%) as a white solid. ¹H NMR (400 MHz, CDCl₃) δ 8.86 (t, J=1.9 Hz, 1H), 8.57 (d, J=7.2 Hz, 1H), 8.12 (d, J=8.0 Hz, 1H), 7.85 (d, J=7.6 Hz, 1H), 7.64 (t, J=7.8 Hz, 1H), 7.19 (t, J=7.6 Hz, 1H), 7.03 (d, J=7.6 Hz, 2H), 6.89 (d, J=7.1 Hz, 1H), 6.50 (s, 1H), 6.18 (s, 1H), 5.38 (dd, J=11.3, 4.4 Hz, 1H), 5.30 (d, J=15.7 Hz, 1H), 4.44-4.34 (m, 1H), 4.16-4.00 (m, 2H), 3.17 (hept, J=6.9 Hz, 1H), 1.97 (s, 6H), 1.78 (dd, J=14.9, 2.2 Hz, 1H), 1.46 (dd, J=15.3, 10.3 Hz, 1H), 1.37 (d, J=6.9 Hz, 6H), 0.51 (s, 3H), 0.38-0.30 (m, 1H), 0.30-0.21 (m, 1H), 0.17-0.09 (m, 1H), 0.05-0.01 (m, 1H). ESI-MS m/z calc. 665.27844, found 666.2 (M+1)⁺; Retention time: 1.8 minutes. LC method A.

### Example 116: Preparation of Compound IV-51

### Step 1: 3-[tert-Butyl(diphenyl)silyl]oxy-2-methyl-propan-1-ol

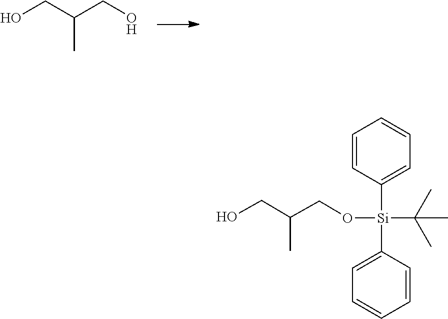

To a solution of 2-methylpropane-1,3-diol (2 g, 22.192 mmol) in dry THE (40 mL) at 0° C. was added NaH (dispersion in mineral oil) (1 g, 60% w/w, 25.002 mmol). The mixture was stirred at 0° C. for 2 minutes and then at room temperature for 1 hour. tert-Butyl(chloro)diphenylsilane (6.5 g, 23.648 mmol) was then added and the mixture was stirred at room temperature for 2 hours. The suspension was cooled down to 0° C. and water (25 mL) was slowly added. The mixture was stirred at room temperature for 15 minutes. EtOAc (100 mL) were added, and the mixture was extracted with EtOAc (3×100 mL). The combined organic layers were washed with a saturated solution of sodium bicarbonate (100 mL), water (100 mL) and brine (100 mL), dried over sodium sulfate, filtered and concentrated in vacuo to afford the crude 3-[tert-butyl(diphenyl)silyl]oxy-2-methyl-propan-1-ol (8.2 g, 108%) as a yellow oil. ¹H NMR (400 MHz, CDCl₃) δ 7.71-7.66 (m, 4H), 7.49-7.38 (m, 6H), 3.77-3.57 (m, 4H), 2.51 (br. s, 1H), 2.07-1.95 (m, 1H), 1.10-1.04 (m, 9H), 0.84 (d, J=6.8 Hz, 3H). ESI-MS m/z calc. 328.1859, found 329.2 (M+1)⁺; Retention time: 2.17 minutes. LC method I.

### Step 2: 3-[tert-Butyl(diphenyl)silyl]oxy-2-methyl-propanal

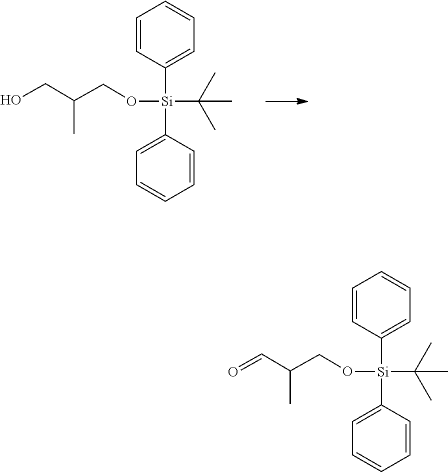 [SMILES: CC(CO)CO[Si](c1ccccc1)(c1ccccc1)C(C)(C)C ; CC(C=O)CO[Si](c1ccccc1)(c1ccccc1)C(C)(C)C]

To a stirred solution of DMSO (5.5050 g, 5 mL, 70.456 mmol) in dry DCM (60 mL) at −78° C. was added oxalyl chloride (4.3650 g, 3 mL, 34.390 mmol). The mixture was stirred at −78° C. for 30 minutes and then a solution of 3-[tert-butyl(diphenyl)silyl]oxy-2-methyl-propan-1-ol (6 g, 17.588 mmol) in DCM (15 mL) was added. The mixture was stirred at −78° C. for 30 minutes and then triethylamine (9.0750 g, 12.5 mL, 89.683 mmol) was added. The mixture was stirred at −78° C. for 30 minutes and then at room temperature for 1 hour. Water (100 mL) and was added and the mixture was extracted with DCM (3×100 mL). The combined organic layers were washed with water (3×250 mL) and brine (3×250 mL), dried over sodium sulfate, filtered and concentrated in vacuo to afford the crude 3-[tert-butyl(diphenyl)silyl]oxy-2-methyl-propanal (6.65 g, 104%) as a yellow oil. ¹H NMR (400 MHz, CDCl₃) δ 9.78 (d, J=1.7 Hz, 1H), 7.67-7.63 (m, 4H), 7.48-7.37 (m, 6H), 3.94-3.81 (m, 2H), 2.63-2.53 (m, 1H), 1.11 (d, J=6.8 Hz, 3H), 1.05 (s, 9H).

### Step 3: tert-Butyl-(4,4-dibromo-2-methyl-but-3-enoxy)-diphenyl-silane

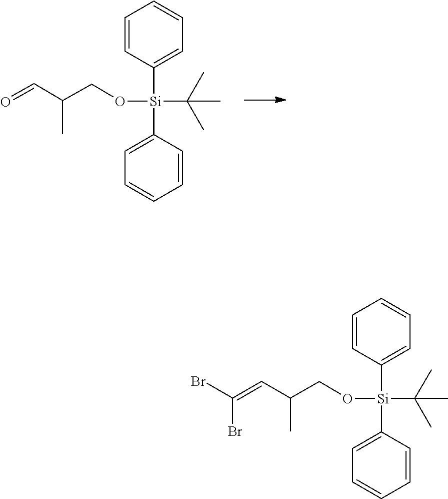 [SMILES: CC(C=O)CO[Si](c1ccccc1)(c1ccccc1)C(C)(C)C ; CC(C=C(Br)Br)CO[Si](c1ccccc1)(c1ccccc1)C(C)(C)C]

To a stirred solution of 3-[tert-butyl(diphenyl)silyl]oxy-2-methyl-propanal (6.6 g, 18.193 mmol) in DCM (150 mL) at 0° C. was added triphenylphosphine (19 g, 72.440 mmol) followed by the slow addition of carbon tetrabromide (12 g, 36.185 mmol). The mixture was stirred at 0° C. for 5 minutes and then at room temperature for 30 minutes. The solution was concentrated in vacuo and the crude mixture was purified by flash-chromatography on a silica gel cartridge, eluting with a gradient of 0 to 5% of EtOAc in heptane to afford after evaporation tert-butyl-(4,4-dibromo-2-methyl-but-3-enoxy)-diphenyl-silane (6.1 g, 66%) as a yellow oil. ¹H NMR (400 MHz, CDCl₃) δ 7.69-7.65 (m, 4H), 7.46-7.37 (m, 6H), 6.28 (d, J=9.3 Hz, 1H), 3.60-3.52 (m, 2H), 2.76-2.67 (m, 1H), 1.07 (s, 9H), 1.05 (d, J=6.8 Hz, 3H).

### Step 4: tert-Butyl-(2-methylbut-3-ynoxy)-diphenyl-silane

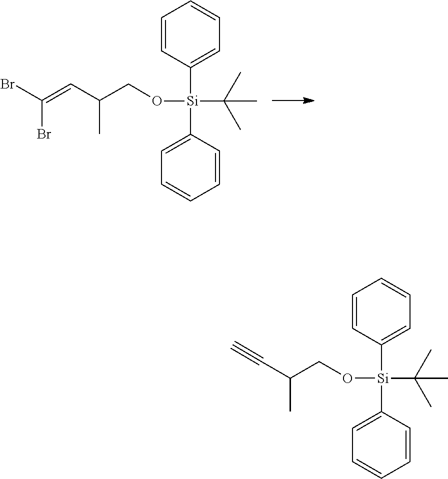 [SMILES: CC(C=C(Br)Br)CO[Si](c1ccccc1)(c1ccccc1)C(C)(C)C ; C#CC(C)CO[Si](c1ccccc1)(c1ccccc1)C(C)(C)C]

To a stirred solution of tert-butyl-(4,4-dibromo-2-methyl-but-3-enoxy)-diphenyl-silane (6 g, 11.818 mmol) in dry THE (125 mL) at −78° C. was added n-BuLi (in hexanes solution) (12 mL of 2.5 M, 30.000 mmol). The mixture was stirred at −78° C. for 30 minutes and then at room temperature for 30 minutes. Water (100 mL) was added, and the mixture was extracted with EtOAc (3×100 mL). The combined organic layers were washed with brine (250 mL), dried over sodium sulfate, filtered, and concentrated in vacuo to afford the crude tert-butyl-(2-methylbut-3-ynoxy)-diphenyl-silane (4.3 g, 102%) as a pale-yellow oil. ¹H NMR (400 MHz, CDCl₃) δ 7.71-7.67 (m, 4H), 7.45-7.38 (m, 6H), 3.78-3.71 (m, 1H), 3.58-3.52 (m, J=9.5 Hz, 1H), 2.72-2.60 (m, 1H), 2.04 (d, J=2.4 Hz, 1H), 1.24 (d, J=7.1 Hz, 3H), 1.08 (s, 9H).

### Step 5: 3-[4-[tert-Butyl(diphenyl)silyl]oxy-3-methyl-but-1-ynyl]-5-chloro-1-[(4-methoxyphenyl)methyl]pyrazin-2-one

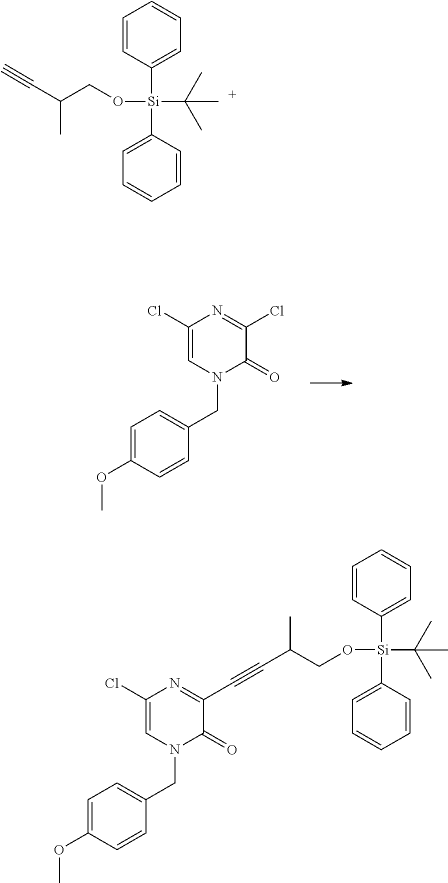

To a stirred solution of 3,5-dichloro-1-[(4-methoxyphenyl)methyl]pyrazin-2-one (3 g, 9.9956 mmol) in a mixture of triethylamine (15 mL) and DMF (15 mL) was added tert-butyl-(2-methylbut-3-ynoxy)-diphenyl-silane (5 g, 13.953 mmol). Nitrogen was bubbled into the mixture for 15 minutes and then bis(triphenylphosphine)palladium(II) dichloride (750 mg, 1.0685 mmol) followed by copper iodide (380 mg, 1.9953 mmol) was added. Nitrogen was bubbled into the mixture for 5 minutes. The mixture was stirred at 40° C. for 30 minutes and was then cooled down to room temperature. Water (250 mL) and EtOAc (250 mL) were added, and the mixture was extracted with EtOAc (3×250 mL). The combined organic layers were washed with water (3×250 mL) and brine (3×250 mL), dried over sodium sulfate, filtered, and concentrated in vacuo. The crude mixture was purified by flash-chromatography on a silica gel cartridge, eluting with a gradient of 0 to 50% of EtOAc in heptane to afford after evaporation 3-[4-[tert-butyl(diphenyl)silyl]oxy-3-methyl-but-1-ynyl]-5-chloro-1-[(4-methoxyphenyl)methyl]pyrazin-2-one (4.8 g, 80%) as a yellow solid. ¹H NMR (400 MHz, CDCl₃) δ 7.71-7.65 (m, 4H), 7.45-7.36 (m, 6H), 7.29-7.25 (m, 2H, overlapped with CDCl₃), 7.11 (s, 1H), 6.93-6.87 (m, 2H), 4.99 (s, 2H), 3.89 (dd, J=9.5, 5.4 Hz, 1H), 3.82 (s, 3H), 3.65 (dd, J=9.5, 8.3 Hz, 1H), 3.06-2.95 (m, 1H), 1.37 (d, J=7.1 Hz, 3H), 1.07 (s, 9H).

### Step 6: tert-Butyl-[2-(2-chlorofuro[2,3-b]pyrazin-6-yl)propoxy]-diphenyl-silane

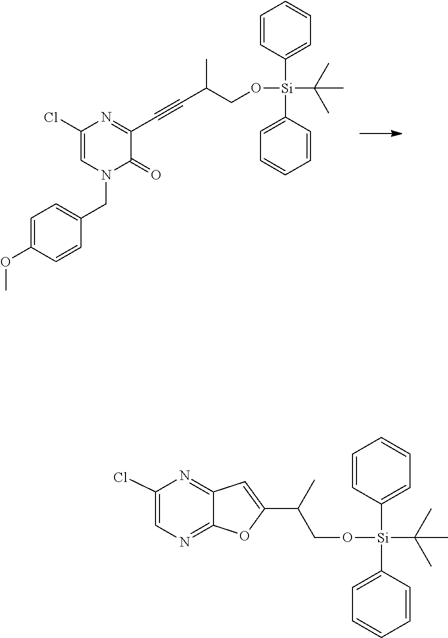 [SMILES: COc1ccc(Cn2cc(Cl)nc(C#CC(C)CO[Si](c3ccccc3)(c3ccccc3)C(C)(C)C)c2=O)cc1 ; CC(CO[Si](c1ccccc1)(c1ccccc1)C(C)(C)C)c1cc2nc(Cl)cnc2o1]

To a stirred solution of 3-[4-[tert-butyl(diphenyl)silyl]oxy-3-methyl-but-1-ynyl]-5-chloro-1-[(4-methoxyphenyl)methyl]pyrazin-2-one (4.8 g, 7.9835 mmol) in DCM (100 mL) was added AgOTf (125 mg, 0.4865 mmol) followed by TFA (9.6200 g, 6.5 mL, 84.369 mmol). The mixture was stirred 45 minutes at room temperature and was then concentrated in vacuo. The crude mixture was purified by flash-chromatography on a silica gel cartridge, eluting with a gradient of 0 to 50% of EtOAc in heptane to afford after evaporation tert-butyl-[2-(2-chlorofuro[2,3-b]pyrazin-6-yl)propoxy]-diphenyl-silane (2.85 g, 76%) as a yellow oil. ¹H NMR (400 MHz, CDCl₃) δ 8.18 (s, 1H), 7.59-7.54 (m, 4H), 7.44-7.37 (m, 2H), 7.37-7.30 (m, 4H), 6.69 (d, J=0.7 Hz, 1H), 3.98-3.89 (m, 2H), 3.30-3.20 (m, 1H), 1.41 (d, J=7.1 Hz, 3H),1.01 (s, 9H). ESI-MS m/z calc. 450.153, found 451.0 (M+1)⁺; Retention time: 2.49 minutes. LC method I.

### Step 7: Methyl 6-[2-[tert-butyl(diphenyl)silyl]oxy-1-methyl-ethyl]furo[2,3-b]pyrazine-2-carboxylate

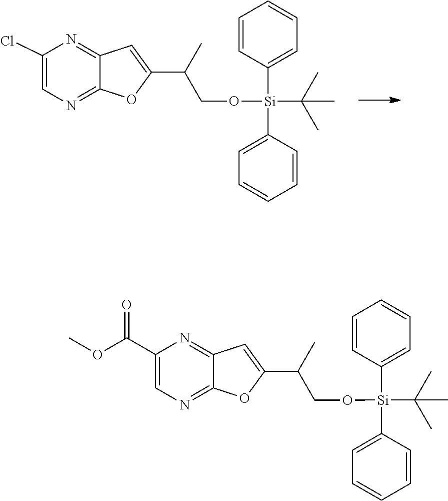

To a solution of tert-butyl-[2-(2-chlorofuro[2,3-b]pyrazin-6-yl)propoxy]-diphenyl-silane (1.35 g, 2.8674 mmol) in MeOH (25 mL) was added triethylamine (726.00 mg, 1 mL, 7.1746 mmol). Nitrogen was bubbled into the mixture for 5 minutes and then 1,1′-bis(diphenylphosphino)ferrocene palladium(II) chloride, complex with dichloromethane (125 mg, 0.1531 mmol) was added. Nitrogen was bubbled into the mixture for 2 minutes. The mixture was then stirred at 100° C. under 50 psi carbon monoxide pressure for 18 hours. The mixture was then cooled down to room temperature. Water (100 mL) and EtOAc (100 mL) were added, and the mixture was extracted with EtOAc (3×100 mL). The combined organic layers were washed with brine (1×250 mL), dried over sodium sulfate, filtered, and concentrated in vacuo. A solution of EtOAc in heptane (50%, 500 mL) was added and the mixture was filtered on a pad of silica. The mixture was concentrated in vacuo to afford crude methyl 6-[2-[tert-butyl(diphenyl)silyl]oxy-1-methyl-ethyl]furo[2,3-b]pyrazine-2-carboxylate (1.4 g, 100%) as a brown oil. ESI-MS m/z calc. 474.1975, found 475.2 (M+1)⁺; Retention time: 2.29 minutes. LC method I.

### Step 8: 6-[2-[tert-Butyl(diphenyl)silyl]oxy-1-methyl-ethyl]furo[2,3-b]pyrazine-2-carbaldehyde

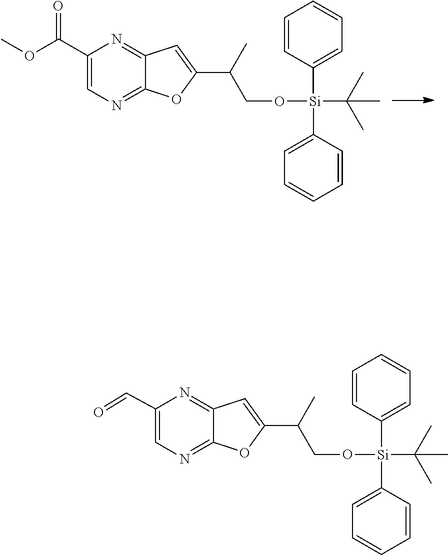 [SMILES: COC(=O)c1cnc2oc(C(C)CO[Si](c3ccccc3)(c3ccccc3)C(C)(C)C)cc2n1 ; CC(CO[Si](c1ccccc1)(c1ccccc1)C(C)(C)C)c1cc2nc(C=O)cnc2o1]

The solution of methyl 6-[2-[tert-butyl(diphenyl)silyl]oxy-1-methyl-ethyl]furo[2,3-b]pyrazine-2-carboxylate (500 mg, 1.0524 mmol) in dry THE (10 mL) was stirred at −78° C. for 15 minutes, then DIBAL-H (methylene chloride solution) (2.5 mL of 1 M, 2.5000 mmol) was added dropwise over 15 minutes. The mixture was stirred at −78° C. for 2 hours and quenched with MeOH (20 mL) and a saturated solution of ammonium chloride (20 mL). The organic solvent was evaporated in vacuo and the residue was diluted with a saturated solution of ammonium chloride (50 mL). The aqueous layer was extracted with EtOAc (3×50 mL) and the combined organic layers were washed with brine (50 mL), dried over sodium sulfate, filtered and concentrated in vacuo to afford crude 6-[2-[tert-butyl(diphenyl)silyl]oxy-1-methyl-ethyl]furo[2,3-b]pyrazine-2-carbaldehyde (495 mg, 102%) as a yellow oil. ¹H NMR (400 MHz, CDCl₃) δ 10.21 (s, 1H), 8.87 (s, 1H), 7.60-7.54 (m, 4H), 7.44-7.37 (m, 2H), 7.36-7.29 (m, 4H), 6.83 (s, 1H), 4.01-3.92 (m, 2H), 3.34-3.24 (m, 1H), 1.44 (d, J=7.1 Hz, 3H), 1.01 (s, 9H). ESI-MS m/z calc. 444.1869, found 445.2 (M+1)⁺; Retention time: 2.31 minutes. LC method I.

### Step 9: 3-[[4-[(2R)-2-[[6-[2-[tert-Butyl(diphenyl)silyl]oxy-1-methyl-ethyl]furo[2,3-b]pyrazin-2-yl]methylamino]-3-(1-methylcyclopropyl)propoxy]-6-(2,6-dimethylphenyl)pyrimidin-2-yl]sulfamoyl]benzoic acid

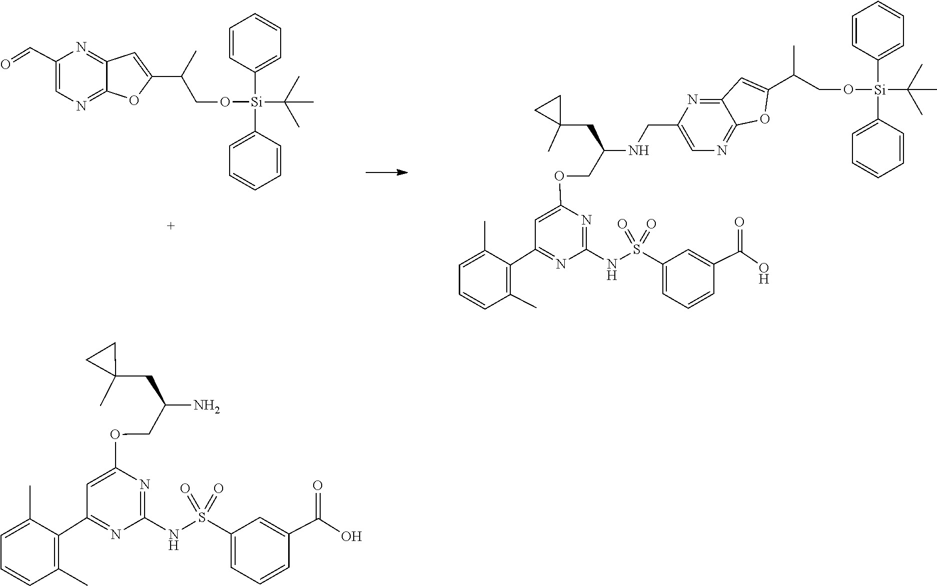 [SMILES: CC(CO[Si](c1ccccc1)(c1ccccc1)C(C)(C)C)c1cc2nc(C=O)cnc2o1 ; Cc1cccc(C)c1-c1cc(OC[C@@H](CC2(C)CC2)NCc2cnc3oc(C(C)CO[Si](c4ccccc4)(c4ccccc4)C(C)(C)C)cc3n2)nc(NS(=O)(=O)c2cccc(C(=O)O)c2)n1 ; Cc1cccc(C)c1-c1cc(OC[C@H](N)CC2(C)CC2)nc(NS(=O)(=O)c2cccc(C(=O)O)c2)n1]

To a 0° C. solution of 6-[2-[tert-butyl(diphenyl)silyl]oxy-1-methyl-ethyl]furo[2,3-b]pyrazine-2-carbaldehyde (500 mg, 1.0853 mmol) and 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (500 mg, 0.9131 mmol) in dry dichloromethane (50 mL) was added sodium triacetoxyborohydride (800 mg, 3.7746 mmol). The mixture was stirred at 0° C. for 90 minutes. The reaction was then quenched at 0° C. by the addition of an aqueous hydrochloric acid solution (1N, 25 mL) and the mixture was stirred for 5 minutes at 0° C. The mixture was then concentrated in vacuo. The crude mixture was purified by reverse phase chromatography on a C₁₈ cartridge, eluting with a gradient of 5 to 100% of MeCN in acidic water (0.1% of hydrochloric acid in water). The fractions containing the desired product were combined and the organic solvent was evaporated. EtOAc (100 mL) was then added, and the mixture was extracted with EtOAc (3×100 mL). The combined organic layers were washed with brine (1×100 mL), dried over sodium sulfate, filtered and concentrated in vacuo to afford 3-[[4-[(2R)-2-[[6-[2-[tert-butyl(diphenyl)silyl]oxy-1-methyl-ethyl]furo[2,3-b]pyrazin-2-yl]methylamino]-3-(1-methylcyclopropyl)propoxy]-6-(2,6-dimethylphenyl)pyrimidin-2-yl]sulfamoyl]benzoic acid (hydrochloride salt) (405 mg, 45%) as a beige solid. ¹H NMR (400 MHz, DMSO-d6) δ 13.33 (br. s, 1H), 12.08 (br. s, 1H), 9.51 (br. s, 2H), 8.47 (s, 2H), 8.19-8.07 (m, 2H), 7.68 (t, J=7.7 Hz, 1H), 7.52-7.45 (m, 4H), 7.44-7.39 (m, 2H), 7.37-7.29 (m, 4H), 7.28-7.20 (m, 1H), 7.12 (d, J=7.6 Hz, 2H), 7.02 (s, 1H), 6.32 (br. s, 1H), 4.76-4.65 (m, 1H), 4.60 (br. s, 2H), 4.43-4.28 (m, 1H), 3.99-3.93 (m, 1H), 3.93-3.87 (m, 1H), 3.78 (br. s, 1H), 3.41-3.27 (overlapped with water, m, 1H), 2.21-2.11 (m, 1H), 1.99 (s, 6H), 1.49-1.40 (m, 1H), 1.37 (d, J=7.1 Hz, 3H), 1.00 (s, 3H), 0.89 (s, 9H), 0.38-0.32 (m, 1H), 0.32-0.25 (m, 1H), 0.24-0.13 (m, 2H). ESI-MS m/z calc. 938.3857, found 939.0 (M+1)⁺; Retention time: 1.95 minutes. LC method I.

### Step 10: (11R)-12-[[6-[2-[tert-Butyl(diphenyl)silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18), 4(19), 5,7,14,16-hexaen-13-one

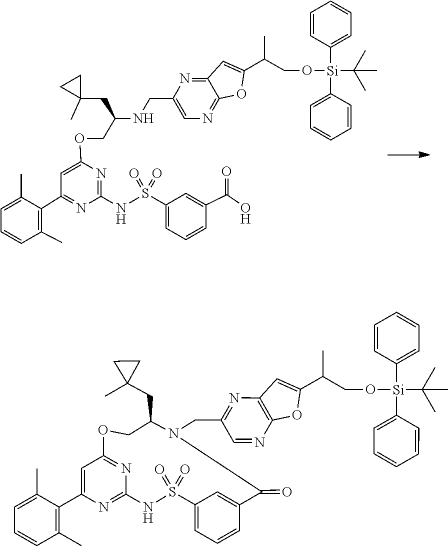 [SMILES: Cc1cccc(C)c1-c1cc(OC[C@@H](CC2(C)CC2)NCc2cnc3oc(C(C)CO[Si](c4ccccc4)(c4ccccc4)C(C)(C)C)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(C(C)CO[Si](c4ccccc4)(c4ccccc4)C(C)(C)C)cc3n1)[C@H](CC1(C)CC1)CO2]

To a solution of 3-[[4-[(2R)-2-[[6-[2-[tert-butyl(diphenyl)silyl]oxy-1-methyl-ethyl]furo[2,3-b]pyrazin-2-yl]methylamino]-3-(1-methylcyclopropyl)propoxy]-6-(2,6-dimethylphenyl)pyrimidin-2-yl]sulfamoyl]benzoic acid (hydrochloride salt) (400 mg, 0.4096 mmol) in DMF (50 mL) at 0° C. was added N-methylmorpholine (552.00 mg, 0.6 mL, 5.4574 mmol) followed by 2-chloro-4,6-dimethoxy-1,3,5-triazine (150 mg, 0.8543 mmol). The mixture was stirred at 0° C. for 15 minutes and then stirred at room temperature for 18 hours. Water (100 mL) and EtOAc (100 mL) were added, and the mixture was extracted with EtOAc (3×100 mL). The combined organic layers were washed with water (2×250 mL) and brine (2×250 mL), dried over sodium sulfate, filtered, and concentrated in vacuo. The crude mixture was purified by reverse phase chromatography on a C₁₈ cartridge, eluting with a gradient of 50 to 100% of MeCN in acidic water (0.1% v/v of formic acid in water). The fractions containing the desired product were combined and the organic solvent was evaporated. EtOAc (50 mL) was then added, and the mixture was extracted with EtOAc (1×50 mL). The combined organic layers were washed with brine (1×50 mL), dried over sodium sulfate, filtered and concentrated in vacuo to afford (11R)-12-[[6-[2-[tert-butyl(diphenyl)silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (280 mg, 70%) as a beige solid. ¹H NMR (400 MHz, CDCl₃) δ 8.72 (d, J=1.7 Hz, 1H), 8.44 (s, 1H), 8.07 (d, J=7.8 Hz, 1H), 7.87 (d, J=7.6 Hz, 1H), 7.67-7.56 (m, 5H), 7.43-7.30 (m, 6H), 7.24-7.19 (m, 1H), 7.07 (d, J=7.6 Hz, 2H), 6.70 (s, 1H), 6.25 (s, 1H), 5.38 (dd, J=11.2, 4.2 Hz, 1H), 5.30 (d, J=14.7 Hz, 1H), 4.48-4.38 (m, 1H), 4.35-4.25 (m, 1H), 4.21-4.09 (overlapped with EtOAc, m, 1H), 4.05-3.96 (m, 1H), 3.94-3.84 (m, 1H), 3.32-3.19 (m, 1H), 2.01 (s, 6H), 1.82 (d, J=14.4 Hz, 1H), 1.50 (dd, J=15.2, 10.0 Hz, 1H), 1.43 (dd, J=10.4, 7.0 Hz, 3H), 1.02 (s, 9H), 0.49 (s, 3H), 0.39-0.32 (m, 1H), 0.28-0.22 (m, 1H), 0.16-0.10 (m, 1H), 0.05-−0.02 (m, 1H). 1 missing labile proton.

### Step 11: (11R)-12-[[6-[(1S)-2-[tert-Butyl(diphenyl)silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one and (11R)-12-[[6-[(JR)-2-[tert-butyl(diphenyl)silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

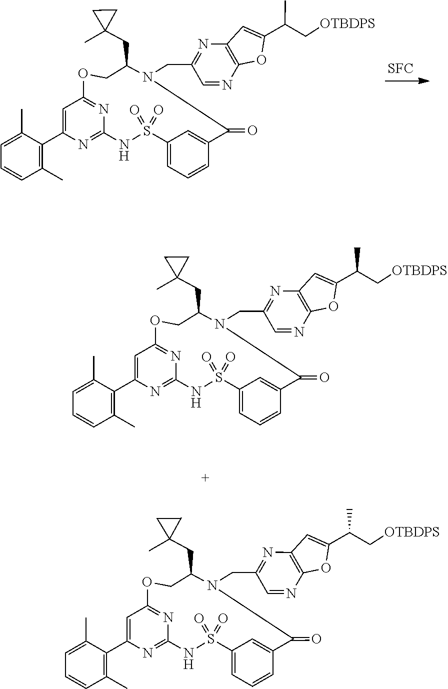

Two batches of (11R)-12-[[6-[2-[tert-butyl(diphenyl)silyl]oxy-1-methyl-ethyl]furo[2,3-b]pyrazin-2-yl]methyl]-6-(2,6-dimethylphenyl)-11-[(1-methylcyclo propyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (205 mg, 0.2223 mmol) and (11R)-12-[[6-[2-[tert-butyl(diphenyl)silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (38 mg, 0.0412 mmol) were dissolved in DCM (5 mL). The solvent was concentrated in vacuo to obtain the desired product as a diastereomeric mixture (209 mg, 86%) as a colorless oil. The mixture was submitted to chiral SFC separation (Flow rate: 100 mL/min, 45% MeOH, column: Cellulose 5-30 mm, temperature=40° C., outlet pressure: 100 bar, injection volume: 300 μL). Fastest eluting peak by SFC gave after evaporation to dryness and lyophilization (11R)-12-[[6-[(1S)-2-[tert-butyl(diphenyl)silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (78 mg, 64%) as a white solid. ¹H NMR (400 MHz, CDCl₃) δ 8.76-8.71 (m, 1H), 8.43 (s, 1H), 8.09 (d, J=7.8 Hz, 1H), 7.88 (d, J=7.6 Hz, 1H), 7.65 (t, J=7.8 Hz, 1H), 7.62-7.57 (m, 4H), 7.43-7.37 (m, 2H), 7.36-7.31 (m, 4H), 7.22 (t, J=8.3 Hz, 1H), 7.08 (d, J=7.6 Hz, 2H), 6.70 (s, 1H), 6.27 (s, 1H), 5.38 (dd, J=11.4, 4.3 Hz, 1H), 5.29 (d, J=14.7 Hz, 1H), 4.50-4.41 (m, 1H), 4.30 (t, J=11.5 Hz, 1H), 4.18 (d, J=14.9 Hz, 1H), 4.00 (dd, J=10.0, 6.4 Hz, 1H), 3.86 (dd, J=10.0, 6.4 Hz, 1H), 3.31-3.22 (m, 1H), 2.02 (br. s, 6H), 1.83 (d, J=15.4 Hz, 1H), 1.52-1.47 (overlapped with water, m, 1H), 1.42 (d, J=6.8 Hz, 3H), 1.02 (s, 9H), 0.48 (s, 3H), 0.39-0.32 (m, 1H), 0.28-0.22 (m, 1H), 0.17-0.10 (m, 1H), 0.05-−0.02 (m, 1H), (1H missing, labile proton). ESI-MS m/z calc. 920.3751, no ionization; Retention time: 5.33 minutes. Slowest eluting peak by SFC gave after evaporation to dryness and lyophilization (11R)-12-[[6-[(1R)-2-[tert-butyl(diphenyl) silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (82 mg, 66%) as a white solid. ¹H NMR (400 MHz, CDCl₃) δ 8.76-8.70 (m, 1H), 8.43 (s, 1H), 8.10 (d, J=8.1 Hz, 1H), 7.89 (d, J=7.8 Hz, 1H), 7.69-7.63 (overlapped with impurity, m, 1H), 7.63-7.56 (m, 4H), 7.45-7.30 (overlapped with impurity, m, 6H), 7.24-7.18 (overlapped with CDCl₃, m, 1H), 7.08 (d, J=7.8 Hz, 2H), 6.70 (s, 1H), 6.28 (s, 1H), 5.38 (dd, J=11.4, 4.0 Hz, 1H), 5.29 (d, J=14.9 Hz, 1H), 4.51-4.41 (m, 1H), 4.31 (t, J=11.5 Hz, 1H), 4.19 (d, J=14.4 Hz, 1H), 3.98 (dd, J=10.8, 5.9 Hz, 1H), 3.90 (dd, J=10.0, 6.8 Hz, 1H), 3.29-3.20 (m, 1H), 2.04 (overlapped with impurity, br. s, 6H), 1.83 (d, J=14.7 Hz, 1H), 1.51-1.47 (m, 1H), 1.44 (overlapped with impurity, d, J=7.1 Hz, 3H), 1.02 (overlapped with impurity, s, 9H), 0.47 (s, 3H), 0.40-0.31 (m, 1H), 0.28-0.20 (m, 1H), 0.17-0.09 (m, 1H), 0.05-−0.05 (m, 1H), (1H missing, labile proton). ESI-MS m/z calc. 920.3751, no ionization; Retention time: 5.32 minutes, LC method J.

### Step 12: (11R)-6-(2,6-Dimethylphenyl)-12-[[6-[(1S)-2-hydroxy-1-methyl-ethyl]furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-51

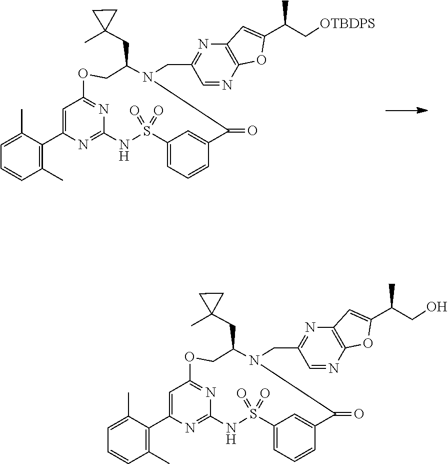

To a stirred solution of (11R)-12-[[6-[(1S)-2-[tert-butyl(diphenyl)silyl]oxy-1-methyl-ethyl]furo[2,3-b]pyrazin-2-yl]methyl]-6-(2,6-dimethylphenyl)-11-[(1-methylcyclo-propyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (58 mg, 0.0629 mmol) in anhydrous THE (1 mL) was added TBAF (in THE solution) (0.200 mL of 1 M, 0.2000 mmol). The mixture was stirred at 0° C. for 30 minutes and then at room temperature for 16 hours. The solvent was removed by reduced pressure and the crude mixture was purified by reverse phase chromatography (Column: C₁₈. Gradient 5-100% of MeCN in water with 0.1% formic acid). The fractions containing the desired product were combined, concentrated in vacuo and coevaporated with MeCN to afford after lyophilization (11R)-6-(2,6-dimethylphenyl)-12-[[6-[(1S)-2-hydroxy-1-methyl-ethyl]furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (25 mg, 58%) as a white solid. ¹H NMR (400 MHz, CDCl₃) δ 8.74 (t, J=1.6 Hz, 1H), 8.45 (s, 1H), 8.08 (dt, J=8.1, 1.5 Hz, 1H), 7.87 (dt, J=7.8, 1.0 Hz, 1H), 7.64 (t, J=7.7 Hz, 1H), 7.22 (t, J=8.6 Hz, 1H), 7.08 (d, J=7.6 Hz, 2H), 6.78 (d, J=0.7 Hz, 1H), 6.26 (s, 1H), 5.41 (dd, J=11.1, 4.3 Hz, 1H), 5.29 (d, J=14.9 Hz, 1H), 4.48-4.39 (m, 1H), 4.31 (t, J=11.7 Hz, 1H), 4.18 (d, J=14.9 Hz, 1H), 4.01-3.88 (m, 2H), 3.33-3.24 (m, 1H), 2.03 (s, 6H), 1.83 (d, J=15.2 Hz, 1H), 1.53-1.47 (m, 1H), 1.44 (d, J=6.8 Hz, 3H), 0.49 (s, 3H), 0.39-0.33 (m, 1H), 0.29-0.23 (m, 1H), 0.18-0.11 (m, 1H), 0.06-−0.01 (m, 1H), (2H missing, labile protons). ESI-MS m/z calc. 682.2574, found 683.2 (M+1)⁺; Retention time: 4.07 minutes. LC method J.

### Example 117: Preparation of Compound IV-52

### Step 1: (11R)-6-(2,6-Dimethylphenyl)-12-[[6-[(1R)-2-hydroxy-1-methyl-ethyl]furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-52

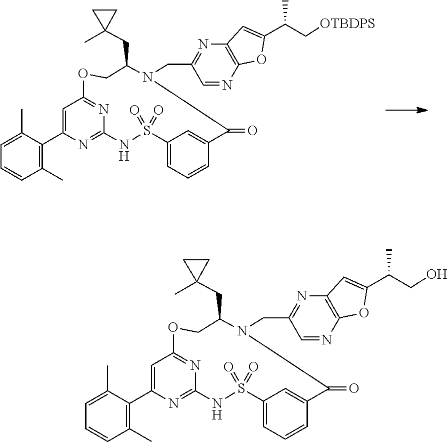

To a stirred solution of (11R)-12-[[6-[(1R)-2-[tert-butyl(diphenyl)silyl]oxy-1-methyl-ethyl]furo[2,3-b]pyrazin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (60 mg, 0.0630 mmol) in dry THE (0.7 mL) was added TBAF (in THE solution) (0.2 mL of 1 M, 0.2000 mmol). The mixture was stirred at 0° C. for 30 minutes and then at room temperature overnight. The mixture was evaporated under reduced pressure and purified by reverse flash chromatography (gradient: 5 to 95% MeCN in an aqueous solution of formic acid (0.1% v/v), 12 CV) the purified fraction was co-evaporated with MeCN (3×5 mL) and lyophilized to afford (11R)-6-(2,6-dimethylphenyl)-12-[[6-[(1R)-2-hydroxy-1-methyl-ethyl]furo[2,3-b]pyrazin-2-yl]methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (23.8 mg, 55%) as a white solid ¹H NMR (400 MHz, CDCl₃) δ 8.74 (t, J=1.0 Hz, 1H), 8.44 (s, 1H), 8.09 (d, J=8.1 Hz, 1H), 7.87 (d, J=7.6 Hz, 1H), 7.64 (t, J=7.8 Hz, 1H), 7.22 (t, J=7.3 Hz, 1H), 7.07 (d, J=7.6 Hz, 2H), 6.78 (s, 1H), 6.26 (s, 1H), 5.41 (dd, J=11.2, 4.4 Hz, 1H), 5.29 (d, J=14.9 Hz, 1H), 4.48-4.39 (m, 1H), 4.30 (t, J=11.5 Hz, 1H), 4.18 (d, J=14.7 Hz, 1H), 4.00-3.89 (m, 2H), 3.34-3.23 (m, 1H), 2.03 (s, 6H), 1.83 (d, J=14.9 Hz, 1H), 1.52-1.48 (m, 1H), 1.44 (d, J=7.1 Hz, 3H), 0.49 (s, 3H), 0.40-0.33 (m, 1H), 0.29-0.23 (m, 1H), 0.18-0.11 (m, 1H), 0.06-−0.01 (m, 1H), (2H missing, labile protons). ESI-MS m/z calc. 682.2574, found 683.3 (M+1)⁺; Retention time: 4.05 minutes. LC method J.

### Example 118: Preparation of Compound IV-53

### Step 1: N-Methoxy-N,3-dimethyl-bicyclo[1.1.1]pentane-1-carboxamide

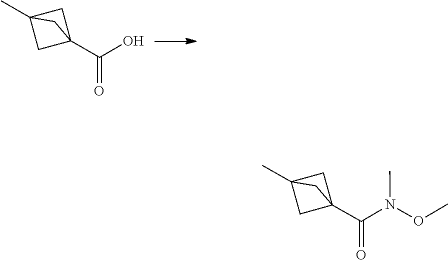

To a solution of 3-methylbicyclo[1.1.1]pentane-1-carboxylic acid (6 g, 46.134 mmol) in DMF (60 mL) at 0° C. was added N-methoxymethanamine; hydrochloride (9.12 g, 93.496 mmol) followed by HATU (21.3 g, 56.019 mmol) and triethylamine (14.520 g, 20 mL, 143.49 mmol). The mixture was stirred at 0° C. for 15 minutes and then at room temperature for 3 hours. Water (100 mL) and EtOAc (100 mL) were added and the mixture was extracted with EtOAc (3×100 mL). The combined organic layers were washed with water (3×250 mL) and brine (3×250 mL), dried over sodium sulfate, filtered, and concentrated in vacuo. The product was dissolved in EtOAc (100 mL) and the organic layer was washed with an aqueous solution of HCl (1N, 100 mL) and a saturated solution of sodium bicarbonate (2×100 mL). The organic layer was dried over sodium sulfate, filtered and concentrated in vacuo to afford N-methoxy-N,3-dimethyl-bicyclo[1.1.1]pentane-1-carboxamide (8.3 g, 90%) as a yellow oil. ¹H NMR (400 MHz, CDCl₃) δ 3.67 (s, 3H), 3.18 (s, 3H), 1.99 (s, 6H), 1.19 (s, 3H). ESI-MS m/z calc. 169.1103, found 170.2 (M+1)⁺; Retention time: 1.57 minutes. LC method I.

### Step 2: 3-Methylbicyclo[1.1.1]pentane-1-carbaldehyde

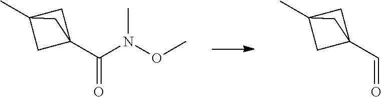

A solution of N-methoxy-N,3-dimethyl-bicyclo[1.1.1]pentane-1-carboxamide (8.3 g, 41.691 mmol) in dry THE (25 mL) at 0° C. was added to a suspension of LAH (2.46 g, 64.815 mmol) in dry THE (75 mL). The mixture was stirred at 0° C. for 15 minutes and then at room temperature for 2 hours. Water (2.5 mL) was added followed by an aqueous solution of NaOH (15%, 2.5 mL) and then water (7.5 mL). The mixture was stirred at room temperature for 30 minutes and magnesium sulfate was added (1 g). The mixture was filtered on Celite and the filter cake was rinsed with THE (100 mL) to afford 3-methylbicyclo[1.1.1]pentane-1-carbaldehyde (4.59 g, 100%) in a THE solution. This solution will be used directly as is in the next reaction as a THE solution.

### Step 3: Methyl 2-(tert-butoxycarbonylamino)-3-(3-methyl-1-bicyclo[1.1.1]pentanyl)prop-2-enoate

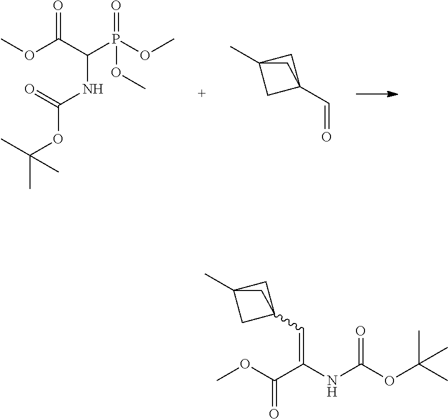

To a stirred solution of 3-methylbicyclo[1.1.1]pentane-1-carbaldehyde (in THE solution) (4.59 g, 41.669 mmol) at 0° C. was added methyl 2-(tert-butoxycarbonylamino)-2-dimethoxyphosphoryl-acetate (4.98 g, 16.754 mmol) followed by DBU (7.6350 g, 7.5 mL, 50.152 mmol). The reaction mixture was stirred at 0° C. for 15 minutes and then at room temperature for 3 hours. Water (100 mL) and EtOAc (100 mL) were added, and the mixture was extracted with EtOAc (3×100 mL). The combined organic layers were washed with brine (250 mL), dried over sodium sulfate, filtered, and concentrated in vacuo. The crude mixture was purified by flash-chromatography on a silica gel cartridge, eluting with a gradient of 0 to 25% of EtOAc in heptane to afford after evaporation methyl 2-(tert-butoxycarbonylamino)-3-(3-methyl-1-bicyclo[1.1.1]pentanyl)prop-2-enoate (4.2 g, 89%) as a white solid. ¹H NMR (400 MHz, CDCl₃) δ 6.42 (br. s, 1H), 5.73 (br. s, 1H), 3.76 (s, 3H), 1.90-1.85 (m, 6H), 1.47 (s, 9H), 1.17 (s, 3H). ESI-MS m/z calc. 281.1627, found 304.2 (M+23)+; Retention time: 1.92 minutes. LC method I.

### Step 4: Methyl (2R)-2-(tert-butoxycarbonylamino)-3-(3-methyl-1-bicyclo[1.1.1]pentanyl)propanoate

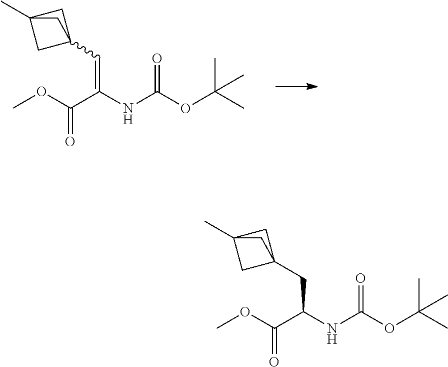 [SMILES: COC(=O)C(=CC12CC(C)(C1)C2)NC(=O)OC(C)(C)C ; COC(=O)[C@@H](CC12CC(C)(C1)C2)NC(=O)OC(C)(C)C]

Methyl 2-(tert-butoxycarbonylamino)-3-(3-methyl-1-bicyclo[1.1.1]pentanyl)prop-2-enoate (4 g, 14.203 mmol) was dissolved in ethanol (30 mL) and dioxane (15 mL). Nitrogen was passed through for 15 minutes and then 1,2-bis[(2R,5R)-2,5-diethylphospholano]benzene(1,5-cyclooctadiene)rhodium(I) trifluoromethanesulfonate (520.6 mg, 0.7204 mmol) was added. Nitrogen was passed through for 5 minutes and then the mixture was hydrogenated under 65 psi hydrogen pressure and at room temperature for 18 hours. The mixture was concentrated in vacuo to dryness and a solution of EtOAc and heptane (25%, 100 mL) was then added. The crude solution was filtered on a silica pad and the pad was rinsed with a solution of EtOAc and heptane (25%, 500 mL) to afford after evaporation methyl (2R)-2-(tert-butoxycarbonylamino)-3-(3-methyl-1-bicyclo[1.1.1]pentanyl)propanoate (3.89 g, 92%) as a yellow oil. ¹H NMR (400 MHz, CDCl₃) δ 4.93 (d, J=7.6 Hz, 1H), 4.37-4.22 (m, 1H), 3.73 (s, 3H), 2.03-1.93 (m, 1H), 1.79 (dd, J=14.5, 7.7 Hz, 1H), 1.56 (s, 6H), 1.45 (s, 9H), 1.12 (s, 3H). ESI-MS m/z calc. 283.1784, found 184.2 (M-99)⁺; Retention time: 1.97 minutes. LC method I.

### Step 5: tert-Butyl N-[(1R)-1-(hydroxymethyl)-2-(3-methyl-1-bicyclo[1.1.1]pentanyl)ethyl]carbamate

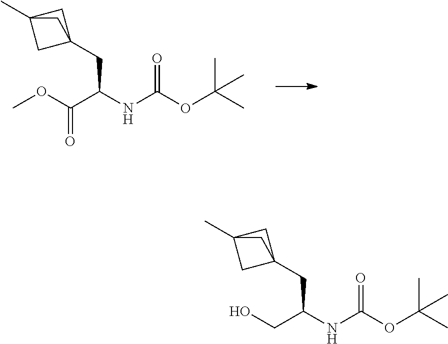 [SMILES: COC(=O)[C@@H](CC12CC(C)(C1)C2)NC(=O)OC(C)(C)C ; CC12CC(C[C@H](CO)NC(=O)OC(C)(C)C)(C1)C2]

To a stirred solution of methyl (2R)-2-(tert-butoxycarbonylamino)-3-(3-methyl-1-bicyclo[1.1.1]pentanyl)propanoate (3.85 g, 12.907 mmol) in a mixture of THF (40 mL) and MeOH (5 mL) at 0° C. was added lithium borohydride (1.43 g, 65.645 mmol). The mixture was stirred at 0° C. for 5 minutes and then at room temperature for 2 hours. The mixture was cooled down to 0° C. and EtOAc (100 mL) was added followed by a saturated solution of ammonium chloride (50 mL). The mixture was stirred at room temperature for 15 minutes. The layers were separated, and the mixture was extracted with EtOAc (3×100 mL). The combined organic layers were washed with a saturated solution of ammonium chloride (2×100 mL) and a saturated solution of sodium bicarbonate (2×100 mL), dried over sodium sulfate, filtered and concentrated in vacuo to afford crude tert-butyl N-[(1R)-1-(hydroxymethyl)-2-(3-methyl-1-bicyclo[1.1.1]pentanyl)ethyl]carbamate (3.25 g, 94%) as a white solid. ¹H NMR (400 MHz, CDCl₃) δ 4.54 (br. s, 1H), 3.71-3.57 (m, 2H), 3.55-3.48 (m, 1H), 2.45 (br. s, 1H), 1.68-1.49 (overlapped with water, m, 8H), 1.46 (s, 9H), 1.14 (s, 3H). ESI-MS m/z calc. 255.1834, found 156.4 (M-99)+; Retention time: 1.8 minutes. LC method I.

### Step 6: (2R)-2-Amino-3-(3-methyl-1-bicyclo[1.1.1]pentanyl)propan-1-ol

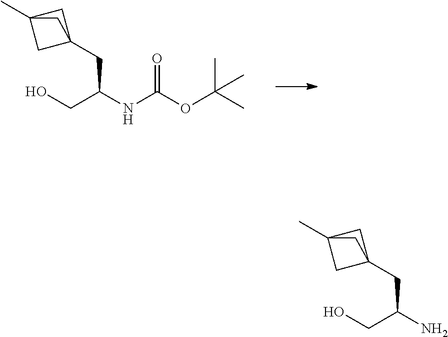 [SMILES: CC12CC(C[C@H](CO)NC(=O)OC(C)(C)C)(C1)C2 ; CC12CC(C[C@@H](N)CO)(C1)C2]

To a stirred solution of tert-butyl N-[(1R)-1-(hydroxymethyl)-2-(3-methyl-1-bicyclo[1.1.1]pentanyl)ethyl]carbamate (215 mg, 0.7999 mmol) in DCM (5 mL) at 0° C. was added HCl (in dioxane solution) (2 mL of 4 M, 8.0000 mmol). The mixture was stirred at 0° C. for 5 minutes and then at room temperature for 6 hours. The mixture was then concentrated in vacuo to afford after co-evaporation with MeCN (3×5 mL) and lyophilization (2R)-2-amino-3-(3-methyl-1-bicyclo[1.1.1]pentanyl)propan-1-ol (hydrochloride salt) (160.3 mg, 99%) as a white solid. ¹H NMR (400 MHz, DMSO-d6) δ 7.90 (br. s, 3H), 5.30 (t, J=5.0 Hz, 1H), 3.64-3.56 (m, 1H), 3.42-3.34 (m, 1H), 2.98 (br. s, 1H), 1.75-1.63 (m, 2H), 1.61-1.51 (m, 6H), 1.11 (s, 3H). ESI-MS m/z calc. 155.131, found 156.2 (M+1)⁺; Retention time: 1.92 minutes. LC method J.

### Step 7: 3-[[4-[(2R)-2-Amino-3-(3-methyl-1-bicyclo[1.1.1]]pentanyl)propoxy]-6-(2,6-dimethylphenyl)pyrimidin-2-yl]sulfamoyl]benzoic acid

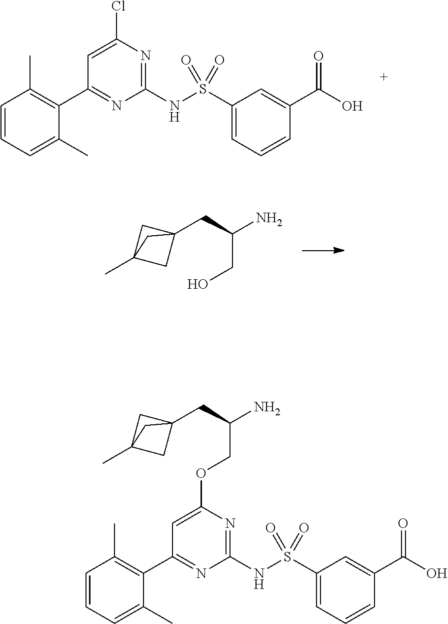 [SMILES: Cc1cccc(C)c1-c1cc(Cl)nc(NS(=O)(=O)c2cccc(C(=O)O)c2)n1 ; CC12CC(C[C@@H](N)CO)(C1)C2 ; Cc1cccc(C)c1-c1cc(OC[C@H](N)CC23CC(C)(C2)C3)nc(NS(=O)(=O)c2cccc(C(=O)O)c2)n1]

In a round bottom flask, 3-[[4-chloro-6-(2,6-dimethylphenyl)pyrimidin-2-yl]sulfamoyl]benzoic acid (255 mg, 0.6102 mmol) and (2R)-2-amino-3-(3-methyl-1-bicyclo[1.1.1]pentanyl)propan-1-ol (hydrochloride salt) (129 mg, 0.6393 mmol) were combined in THE (4 mL). With the suspension stirring in a room temperature water bath, sodium tert-butoxide (352 mg, 3.663 mmol) was added in one portion. The reaction mixture was stirred at room temperature for 2.5 hours. Additional sodium tert-butoxide (70 mg, 0.7284 mmol) and THE (6 mL) were added to reduce viscosity. The reaction mixture was stirred for an additional 1.5 hours. It was cooled to 0° C. in an ice water bath before the addition of aqueous HCl (1 M, 150 mL). After stirring, the mixture was extracted with EtOAc (3×100 mL). Organic layers were combined and concentrated under reduced pressure. The obtained foaming solid was taken up in minimal DCM (approximately 2-3 mL) and triturated with the addition of EtOAc (approximately 5 mL). After briefly stirring for 5 minutes, solids were collected by vacuum filtration. This provided 3-[[4-[(2R)-2-amino-3-(3-methyl-1-bicyclo[1.1.1]pentanyl)propoxy]-6-(2,6-dimethylphenyl)pyrimidin-2-yl]sulfamoyl]benzoic acid (hydrochloride salt) (180 mg, 51%) as a light beige solid. ESI-MS m/z calc. 536.20935, found 537.4 (M+1)⁺; Retention time: 1.03 minutes. LC method A.

### Step 8: 3-[[4-[(2R)-2-[(6-tert-Butylfuro[2,3-b]pyrazin-2-yl)methylamino]-3-(3-methyl-1-bicyclo[1.1.1]pentanyl)propoxy]-6-(2,6-dimethylphenyl)pyrimidin-2-yl]sulfamoyl]benzoic acid

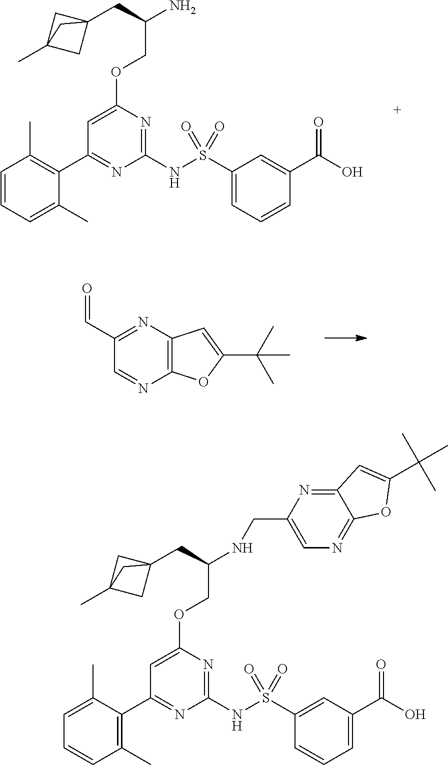 [SMILES: Cc1cccc(C)c1-c1cc(OC[C@H](N)CC23CC(C)(C2)C3)nc(NS(=O)(=O)c2cccc(C(=O)O)c2)n1 ; CC(C)(C)c1cc2nc(C=O)cnc2o1 ; Cc1cccc(C)c1-c1cc(OC[C@@H](CC23CC(C)(C2)C3)NCc2cnc3oc(C(C)(C)C)cc3n2)nc(NS(=O)(=O)c2cccc(C(=O)O)c2)n1]

In a 4 mL vial, 3-[[4-[(2R)-2-amino-3-(3-methyl-1-bicyclo[1.1.1]pentanyl)propoxy]-6-(2,6-dimethylphenyl)pyrimidin-2-yl]sulfamoyl]benzoic acid (hydrochloride salt) (30 mg, 0.05235 mmol) and 6-tert-butylfuro[2,3-b]pyrazine-2-carbaldehyde (approximately 12.29 mg, 0.06020 mmol) were combined in DCM (0.3 mL). Glacial acetic acid (approximately 6.287 mg, 5.954 μL, 0.1047 mmol) was added. The mixture was cooled to 0° C. before the addition of DIEA (approximately 20.29 mg, 27.35 μL, 0.1570 mmol). After stirring for 15 minutes, sodium triacetoxyborohydride (approximately 55.49 mg, 0.2618 mmol) was added. Stirring was continued at 0° C. for 1 hour. The reaction was quenched with the addition of methanol and aqueous 1 M HCl. After bubbling stopped, purification by reverse phase HPLC (10-99% acetonitrile/5 mM aqueous HCl over 15 minutes) provided 3-[[4-[(2R)-2-[(6-tert-butylfuro[2,3-b]pyrazin-2-yl)methylamino]-3-(3-methyl-1-bicyclo[1.1.1]pentanyl)propoxy]-6-(2,6-dimethylphenyl)pyrimidin-2-yl]sulfamoyl]benzoic acid (hydrochloride salt) (23 mg, 58%). ESI-MS m/z calc. 724.3043, found 725.5 (M+1)⁺; Retention time: 1.41 minutes; LC method A.

### Step 9: (11R)-12-[(6-tert-Butylfuro[2,3-b]pyrazin-2-yl)methyl]-6-(2,6-dimethylphenyl)-11-[(3-methyl-1-bicyclo[1.1.1]pentanyl)methyl]-2,2-dioxo-9-oxa-2λ⁶-thia-3,5,12,19-tetrazatri-cyclo[12.3.1.14,8]nonadeca-1(18),4(19),5,7,14,16-hexaen-13-one (Compound IV-53

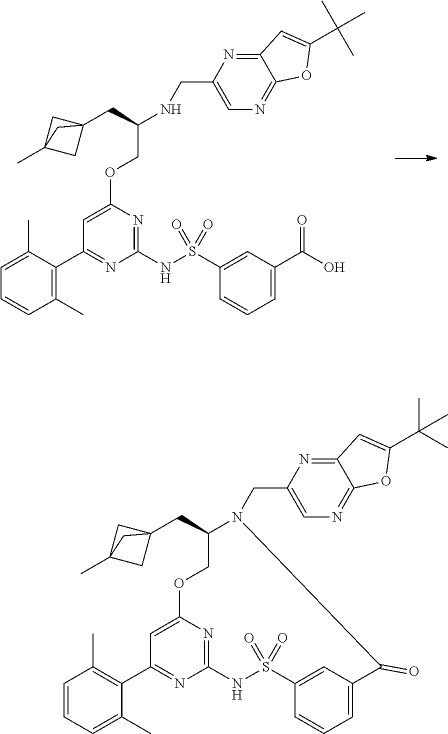 [SMILES: Cc1cccc(C)c1-c1cc(OC[C@@H](CC23CC(C)(C2)C3)NCc2cnc3oc(C(C)(C)C)cc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(C(C)(C)C)cc3n1)[C@H](CC13CC(C)(C1)C3)CO2]

In a 4 mL vial, 3-[[4-[(2R)-2-[(6-tert-butylfuro[2,3-b]pyrazin-2-yl)methylamino]-3-(3-methyl-1-bicyclo[1.1.1]pentanyl)propoxy]-6-(2,6-dimethylphenyl)pyrimidin-2-yl]sulfamoyl]benzoic acid (hydrochloride salt) (23 mg, 0.03021 mmol) and 2-chloro-4,6-dimethoxy-1,3,5-triazine (approximately 6.895 mg, 0.03927 mmol) were combined in DMF. The mixture was cooled to 0° C. before the addition of 4-methylmorpholine (approximately 15.28 mg, 16.61 μL, 0.1511 mmol). The reaction mixture was allowed to warm to room temperature and stirred overnight. Purification by reverse phase HPLC (10-99% acetonitrile/5 mM aqueous HCl over 15 minutes) provided (11R)-12-[(6-tert-butylfuro[2,3-b]pyrazin-2-yl)methyl]-6-(2,6-dimethylphenyl)-11-[(3-methyl-1-bicyclo[1.1.1]pentanyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (9.3 mg, 43%). ESI-MS m/z calc. 706.29376, found 707.6 (M+1)⁺; Retention time: 2.01 minutes; LC method A.

### Example 119: Preparation of Compound IV-54

### Step 1: 6-tert-Butyl-3-chloro-5H-pyrrolo[2,3-b]pyrazine

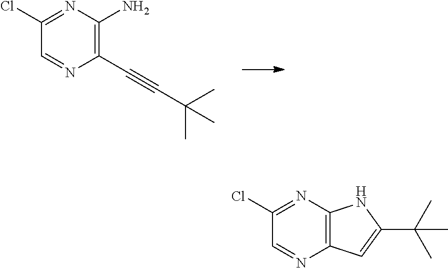

Potassium tert-butoxide (32 g, 285.17 mmol) was added to a solution of 6-chloro-3-(3,3-dimethylbut-1-ynyl)pyrazin-2-amine (25.56 g, 112.88 mmol) in t-butanol (300 mL) and heated at 80° C. for 2 hours. The crude mixture was concentrated under reduced pressure. Water (100 mL) was then added, and the mixture was stirred for 30 minutes. The mixture was extracted with EtOAc (3×100 mL) and the phases were separated. The organic layer was dried over sodium sulfate and evaporated in vacuo, affording 6-tert-butyl-3-chloro-5H-pyrrolo[2,3-b]pyrazine (6.76 g, 28%) as an off-white solid. ¹H NMR (400 MHz, CDCl₃) δ 8.58 (br. s., 1H), 8.38 (s, 1H), 6.45 (d, J=2.2 Hz, 1H), 1.43 (s, 9H). ESI-MS m/z calc. 209.072, found 210.2 (M+1)⁺; Retention time: 1.74 minutes. LC method I.

### Step 2: Methyl 6-tert-butyl-5H-pyrrolo[2,3-b]pyrazine-3-carboxylate

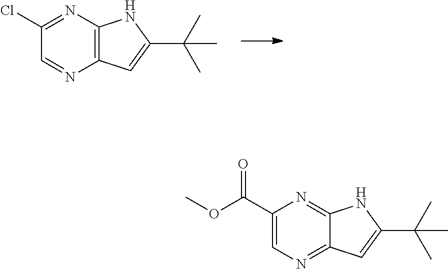 [SMILES: CC(C)(C)c1cc2ncc(Cl)nc2[nH]1 ; COC(=O)c1cnc2cc(C(C)(C)C)[nH]c2n1]

To a suspension of 6-tert-butyl-3-chloro-5H-pyrrolo[2,3-b]pyrazine (6.76 g, 31.273 mmol) in MeOH (80 mL) was added triethylamine (7.2600 g, 10 mL, 71.746 mmol). Nitrogen was bubbled into the mixture for 15 minutes and then 1,1′-bis(diphenylphosphino)ferrocene palladium(II) chloride, complex with dichloromethane (1.27 g, 1.5552 mmol) was added. Nitrogen bubbling was then continued for 5 minutes. The mixture was then stirred at 80° C. under 60 psi carbon monoxide pressure for 18 hours. The mixture was then allowed to cool down to room temperature, was filtered on a pad of Celite and the pad was rinsed with EtOAc (2×100 mL) and concentrated in vacuo. The solution was washed with water (2×100 mL) and brine (100 mL), dried over anhydrous sodium sulfate, filtered, and concentrated in vacuo. The crude solid was triturated in ice-cold MeCN, filtered and rinsed a minimal amount of ice-cold MeCN to afford methyl 6-tert-butyl-5H-pyrrolo[2,3-b]pyrazine-3-carboxylate (5.85 g, 77%) as an off-white solid. ¹H NMR (400 MHz, DMSO-d6) δ 12.43 (br. s., 1H), 8.97 (s, 1H), 6.51 (d, J=2.1 Hz, 1H), 3.91 (s, 3H), 1.40 (s, 9H). ESI-MS m/z calc. 233.1164, found 234.2 (M+1)⁺; Retention time: 1.58 minutes, LC method I.

### Step 3: (6-tert-Butyl-5H-pyrrolo[2,3-b]pyrazin-3-yl)methanol

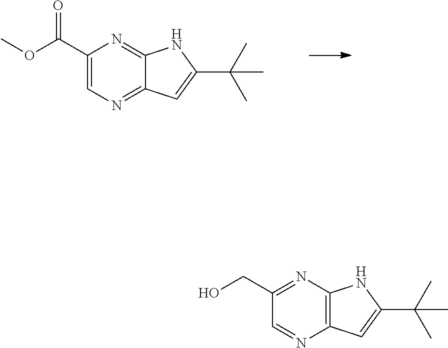 [SMILES: COC(=O)c1cnc2cc(C(C)(C)C)[nH]c2n1 ; CC(C)(C)c1cc2ncc(CO)nc2[nH]1]

To a suspension of methyl 6-tert-butyl-5H-pyrrolo[2,3-b]pyrazine-3-carboxylate (5.65 g, 23.349 mmol) and sodium borohydride (3.32 g, 87.755 mmol) in EtOH (150 mL) was added cerium(III) chloride (Water (7)) (320 mg, 0.8589 mmol) at room temperature, and the reaction was stirred for 3 hours. Then, sodium borohydride (1.5 g, 39.648 mmol) was added, and the reaction was stirred for 1 hour and 30 minutes. The mixture was diluted with a saturated aqueous solution of sodium bicarbonate (50 mL) and stirred for 1 hour. The reaction was concentrated under reduced pressure, and the remaining aqueous phase was extracted with chloroform (5×50 mL), concentrated under reduced pressure, and purified by silica gel chromatography (0-50% EtOAc in heptanes), affording (6-tert-butyl-5H-pyrrolo[2,3-b]pyrazin-3-yl)methanol (2.5 g, 52%) as a white solid. ¹H NMR (400 MHz, DMSO-d6) δ 11.76 (br. s., 1H), 8.37 (s, 1H), 6.28 (d, J=2.1 Hz, 1H), 5.35 (t, J=5.8 Hz, 1H), 4.63 (d, J=5.7 Hz, 2H), 1.37 (s, 9H). ESI-MS m/z calc. 205.1215, found 206.2 (M+1)⁺; Retention time: 1.33 minutes, LC method I.

### Step 4: 6-tert-Butyl-5H-pyrrolo[2,3-b]pyrazine-3-carbaldehyde

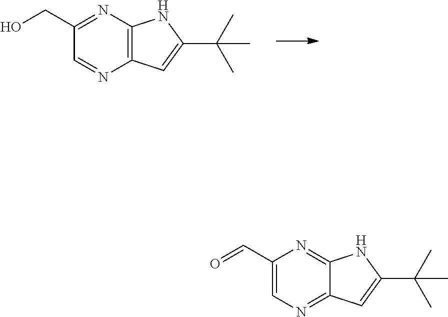

To a suspension of (6-tert-butyl-5H-pyrrolo[2,3-b]pyrazin-3-yl)methanol (2.5 g, 12.168 mmol) in DCM (75 mL) and MeCN (10 mL) was added Dess-Martin periodinane (10 g, 23.577 mmol). The mixture was stirred for 20 minutes at room temperature. Then an aqueous solution of 10% sodium thiosulfate (30 mL) and a saturated aqueous solution of sodium bicarbonate (30 mL) were added, and the mixture was stirred for 1 hour. The mixture was filtered, and the layers were separated. The organic phase was dried over anhydrous sodium sulfate and reunited with the filtered solid. The combined solids were dissolved in DMSO (500 mL) and filtered. The solid was washed with water (2×15 mL), and dried under high vacuum, affording 6-tert-butyl-5H-pyrrolo[2,3-b]pyrazine-3-carbaldehyde (758 mg, 30%) as a white solid. ¹H NMR (400 MHz, Acetone) δ 11.31 (br. s., 1H), 10.03 (s, 1H), 8.91 (s, 1H), 6.58-6.56 (m, 1H), 1.52 (s, 9H). ESI-MS m/z calc. 203.1059, found 204.1 (M+1)⁺; Retention time: 3.33 minutes, LC method J.

### Step 5: 3-[[4-[(2R)-2-[(6-tert-Butyl-5H-pyrrolo[2,3-b]pyrazin-3-yl)methylamino]-3-(1-methylcyclopropyl)propoxy]-6-(2,6-dimethylphenyl)pyrimidin-2-yl]sulfamoyl]benzoic acid

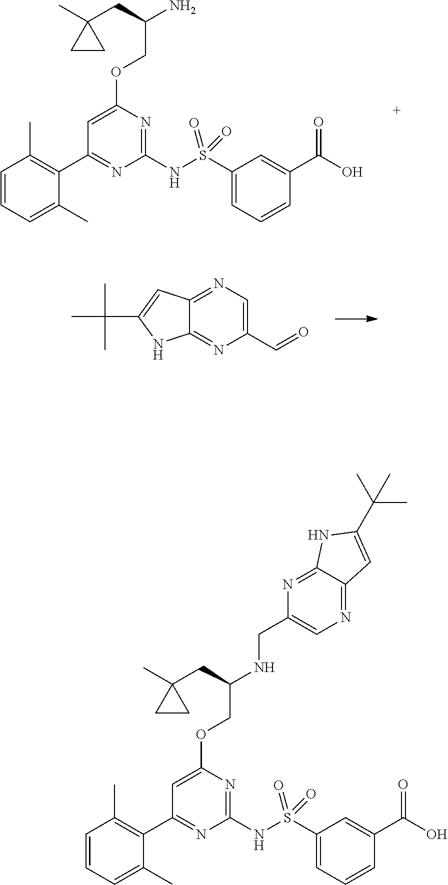

A 4 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (51.5 mg, 0.09414 mmol), 6-tert-butyl-5H-pyrrolo[2,3-b]pyrazine-3-carbaldehyde (20.1 mg, 0.09890 mmol), anhydrous DCM (0.5 mL), and acetic acid (8.0 μL, 0.1407 mmol). The mixture was cooled down in an ice bath. DIEA (32.8 μL, 0.1883 mmol) was added, followed by sodium triacetoxyborohydride (106.7 mg, 0.5034 mmol) and the reaction was vigorously stirred at 0° C. for 2 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-[(2R)-2-[(6-tert-butyl-5H-pyrrolo[2,3-b]pyrazin-3-yl)methylamino]-3-(1-methylcyclopropyl)propoxy]-6-(2,6-dimethylphenyl)pyrimidin-2-yl]sulfamoyl]benzoic acid (hydrochloride salt) (37.1 mg, 54%) as a yellow solid. ESI-MS m/z calc. 697.3046, found 698.4 (M+1)⁺; Retention time: 1.39 minutes. LC method A.

### Step 6: (11R)-12-[(6-tert-Butyl-5H-pyrrolo[2,3-b]pyrazin-3-yl)methyl]-6-(2,6-dimethyl-phenyl)-11-[(I-methylcyclopropyl)methyl]-2,2-dioxo-9-oxa-2λ⁶-thia-3,5,12,19-tetraza-tricyclo[12.3.1.14,8]nonadeca-1(18), 4(19),5,7,14,16-hexaen-13-one (Compound IV-54

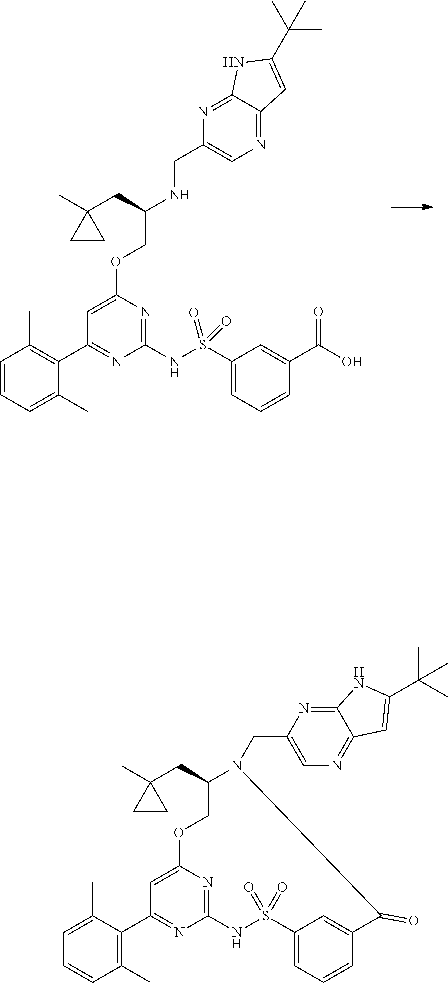

3-[[4-[(2R)-2-[(6-tert-Butyl-5H-pyrrolo[2,3-b]pyrazin-3-yl)methylamino]-3-(1-methylcyclopropyl)propoxy]-6-(2,6-dimethylphenyl)pyrimidin-2-yl]sulfamoyl]benzoic acid (hydrochloride salt) (37.4 mg, 0.05093 mmol) was combined with CDMT (13.4 mg, 0.07632 mmol) in DMF (5.1 mL) and cooled to 0° C. N-methylmorpholine (16.8 μL, 0.1528 mmol) was added by syringe and the reaction was stirred at 0° C. for 30 minutes. The ice bath was then removed and stirring was continued for an additional 5 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was diluted with DMSO (1 mL) and MeOH (1 mL), filtered, and purified by reverse phase HPLC (1-99% acetonitrile/5 mM aqueous HCl over 25 min) to give (11R)-12-[(6-tert-butyl-5H-pyrrolo[2,3-b]pyrazin-3-yl)methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15.3 mg, 44%) as a yellow solid. ¹H NMR (400 MHz, CDCl₃) δ 9.79 (s, 1H), 9.17 (s, 1H), 8.52 (s, 1H), 7.99 (s, 1H), 7.80 (d, J=7.5 Hz, 1H), 7.59 (t, J=7.8 Hz, 1H), 7.22 (t, J=7.6 Hz, 1H), 7.04 (d, J=7.6 Hz, 2H), 6.59 (s, 1H), 6.14 (s, 1H), 5.75-5.64 (m, 1H), 5.45-5.36 (m, 1H), 4.37-4.24 (m, 2H), 4.10-3.95 (m, 1H), 2.01 (s, 6H), 1.87-1.79 (m, 1H), 1.44 (s, 11H), 0.61 (s, 3H), 0.43-0.34 (m, 1H), 0.34-0.24 (m, 1H), 0.21-0.12 (m, 1H), 0.11-0.03 (m, 1H). ESI-MS m/z calc. 679.29407, found 680.6 (M+1)⁺; Retention time: 1.6 minutes. LC method A.

### Example 120: Preparation of Compound IV-55 and Compound IV-56

### Step 1: 2-Amino-3-(3,3-difluorocyclobutyl)propan-1-ol

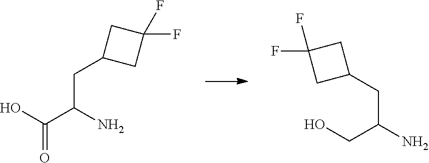 [SMILES: NC(CC1CC(F)(F)C1)C(=O)O ; NC(CO)CC1CC(F)(F)C1]

A solution of 2-amino-3-(3,3-difluorocyclobutyl)propanoic acid (hydrochloride salt) (1.0 g, 4.638 mmol) in THE (10 mL) was cooled to 0° C. before the dropwise addition of borane-tetrahydrofuran complex (16.7 mL of 1 M, 16.70 mmol). The reaction mixture was allowed to warm to room temperature and stirred overnight. The reaction was quenched with the dropwise addition of methanol at 0° C. The mixture was allowed to slowly warm to room temperature and allowed to stir for an additional 2 hours. The reaction was quenched with slow addition of methanol (30 mL) and then co-evaporated with methanol (3×50 mL) and DCE (1×5 mL). The crude material was then suspended in DCM (50 mL) and acidified with HCl in dioxane (approximately 15 mL of 4 M solution). Volatiles were removed under reduced pressure to provide 2-amino-3-(3,3-difluorocyclobutyl)propan-1-ol (hydrochloride salt) (923 mg, 99%). ESI-MS m/z calc. 165.09653, found 166.2 (M+1)⁺; Retention time: 0.18 minutes. LC method B.

### Step 2: 3-[[4-[2-Amino-3-(3,3-difluorocyclobutyl)propoxy]-6-(2,6-dimethylphenyl)pyrimidin-2-yl]sulfamoyl]benzoic acid

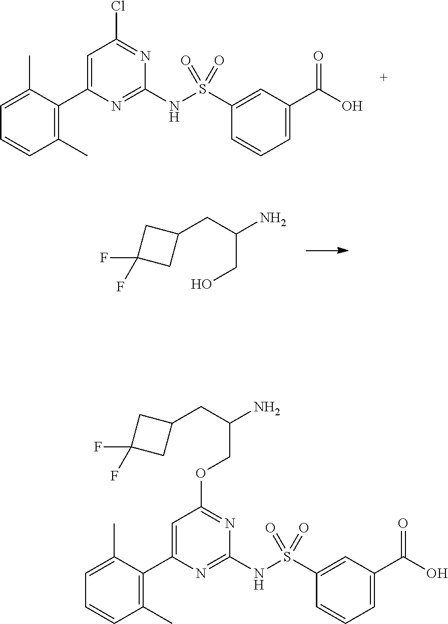 [SMILES: Cc1cccc(C)c1-c1cc(Cl)nc(NS(=O)(=O)c2cccc(C(=O)O)c2)n1 ; NC(CO)CC1CC(F)(F)C1 ; Cc1cccc(C)c1-c1cc(OCC(N)CC2CC(F)(F)C2)nc(NS(=O)(=O)c2cccc(C(=O)O)c2)n1]

In a round bottom flask, 3-[[4-chloro-6-(2,6-dimethylphenyl)pyrimidin-2-yl]sulfamoyl]benzoic acid (500 mg, 1.197 mmol) and 2-amino-3-(3,3-difluorocyclobutyl)propan-1-ol (hydrochloride salt) (266 mg, 1.319 mmol) were combined in THE (7.5 mL). In a room temperature water bath, sodium tert-butoxide (690 mg, 7.180 mmol) was added in one portion. The reaction mixture was stirred for 1.5 hours at room temperature. It was cooled to 0° C. before being quenched with the addition of ice cold aqueous HCl (1 M, 150 mL). After stirring, it was extracted with EtOAc (4×50 mL). Organic layers were combined, washed with brine (1×150 mL), dried over sodium sulfate, filtered, and concentrated under reduced pressure. The obtained solid was taken up in EtOAc/isopropanol (approximately 10 mL at 4:1 ratio) and precipitated with hexanes (approximately 125 mL). Solids were collected by vacuum filtration. 3-[[4-[2-amino-3-(3,3-difluorocyclobutyl)propoxy]-6-(2,6-dimethylphenyl)pyrimidin-2-yl]sulfamoyl]benzoic acid (hydrochloride salt) (587 mg, 84%) was obtained as a beige solid. ESI-MS m/z calc. 546.17487, found 547.4 (M+1)⁺; Retention time: 0.88 minutes. LC method A.

### Step 3: 3-[[4-[2-[(6-tert-Butyl-5-methyl-pyrrolo[2,3-b]pyrazin-3-yl)methylamino]-3-(3,3-difluorocyclobutyl)propoxy]-6-(2,6-dimethylphenyl)pyrimidin-2-yl]sulfamoyl]benzoic acid

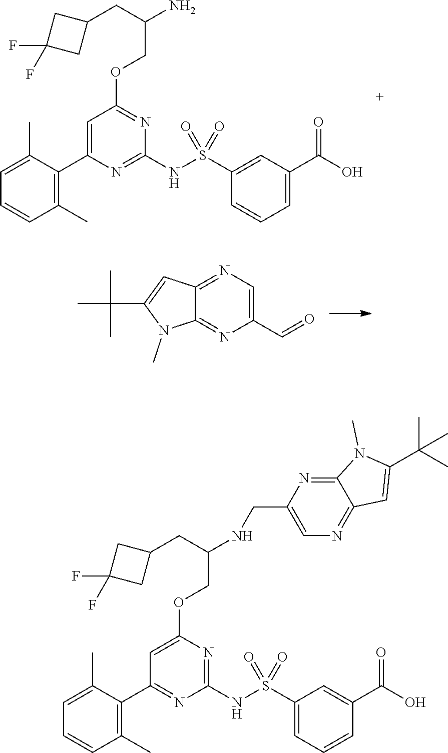 [SMILES: Cc1cccc(C)c1-c1cc(OCC(N)CC2CC(F)(F)C2)nc(NS(=O)(=O)c2cccc(C(=O)O)c2)n1 ; Cn1c(C(C)(C)C)cc2ncc(C=O)nc21 ; Cc1cccc(C)c1-c1cc(OCC(CC2CC(F)(F)C2)NCc2cnc3cc(C(C)(C)C)n(C)c3n2)nc(NS(=O)(=O)c2cccc(C(=O)O)c2)n1]

In a 4 mL vial, 3-[[4-[2-amino-3-(3,3-difluorocyclobutyl)propoxy]-6-(2,6-dimethylphenyl)pyrimidin-2-yl]sulfamoyl]benzoic acid (hydrochloride salt) (35 mg, 0.06003 mmol) and 6-tert-butyl-5-methyl-pyrrolo[2,3-b]pyrazine-3-carbaldehyde (approximately 14.35 mg, 0.06603 mmol) were combined in DCM (0.5 mL). Glacial acetic acid (approximately 7.212 mg, 6.830 μL, 0.1201 mmol) was added. The reaction mixture was cooled to 0° C. before the addition of DIEA (approximately 23.28 mg, 31.37 μL, 0.1801 mmol). After 15 minutes of stirring, sodium triacetoxyborohydride (approximately 63.62 mg, 0.3002 mmol) was added. The reaction mixture was allowed to stir at 0° C. for 1 hour. It was quenched with the addition of methanol and aqueous 1 M HCl. Purification by reverse phase HPLC (10-99% acetonitrile/5 mM aqueous HCl over 15 minutes) provided 3-[[4-[2-[(6-tert-butyl-5-methyl-pyrrolo[2,3-b]pyrazin-3-yl)methylamino]-3-(3,3-difluorocyclobutyl)propoxy]-6-(2,6-dimethylphenyl)pyrimidin-2-yl]sulfamoyl]benzoic acid (hydrochloride salt) (31 mg, 66%). ESI-MS m/z calc. 747.30145, found 748.5 (M+1)⁺; Retention time: 1.24 minutes; LC method A.

### Step 4: 12-[(6-tert-Butyl-5-methyl-pyrrolo[2,3-b]pyrazin-3-yl)methyl]-11-[(3,3-difluorocyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one

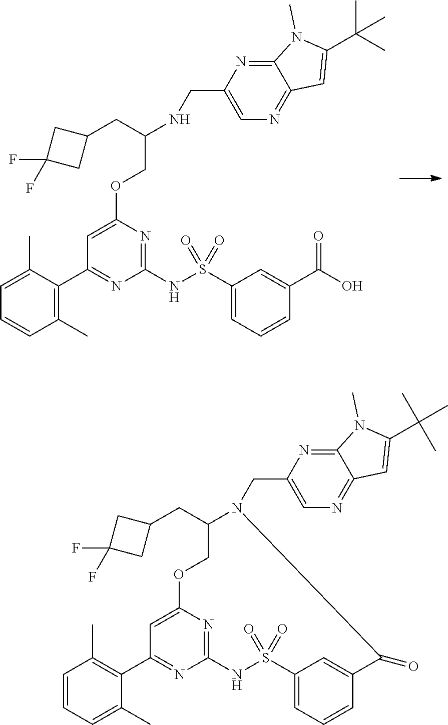 [SMILES: Cc1cccc(C)c1-c1cc(OCC(CC2CC(F)(F)C2)NCc2cnc3cc(C(C)(C)C)n(C)c3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3cc(C(C)(C)C)n(C)c3n1)C(CC1CC(F)(F)C1)CO2]

In a 4 mL vial, 3-[[4-[2-[(6-tert-butyl-5-methyl-pyrrolo[2,3-b]pyrazin-3-yl)methylamino]-3-(3,3-difluorocyclobutyl)propoxy]-6-(2,6-dimethylphenyl)pyrimidin-2-yl]sulfamoyl]benzoic acid (hydrochloride salt) (hydrochloride salt) (31 mg, 0.03952 mmol) and 2-chloro-4,6-dimethoxy-1,3,5-triazine (approximately 10.41 mg, 0.05928 mmol) were combined in DMF. The solution was cooled to 0° C. before the addition of 4-methylmorpholine (approximately 19.99 mg, 21.73 μL, 0.1976 mmol). The reaction mixture was allowed to slowly warm to room temperature and stirred overnight. Purification by reverse phase HPLC (10-99% acetonitrile/5 mM aqueous HCl over 15 minutes) provided 12-[(6-tert-butyl-5-methyl-pyrrolo[2,3-b]pyrazin-3-yl)methyl]-11-[(3,3-difluorocyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (24.4 mg, 85%). ESI-MS m/z calc. 729.2909, found 730.5 (M+1)⁺; Retention time: 1.57 minutes; LC method A.

### Step 5: 12-[(6-tert-Butyl-5-methyl-pyrrolo[2,3-b]pyrazin-3-yl)methyl]-11-[(3,3-difluorocyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, isomer 1, (Compound IV-55), and 12-[(6-tert-butyl-5-methyl-pyrrolo[2,3-b]pyrazin-3-yl)methyl]-11-[(3,3-difluorocyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, isomer 2, (Compound IV-56

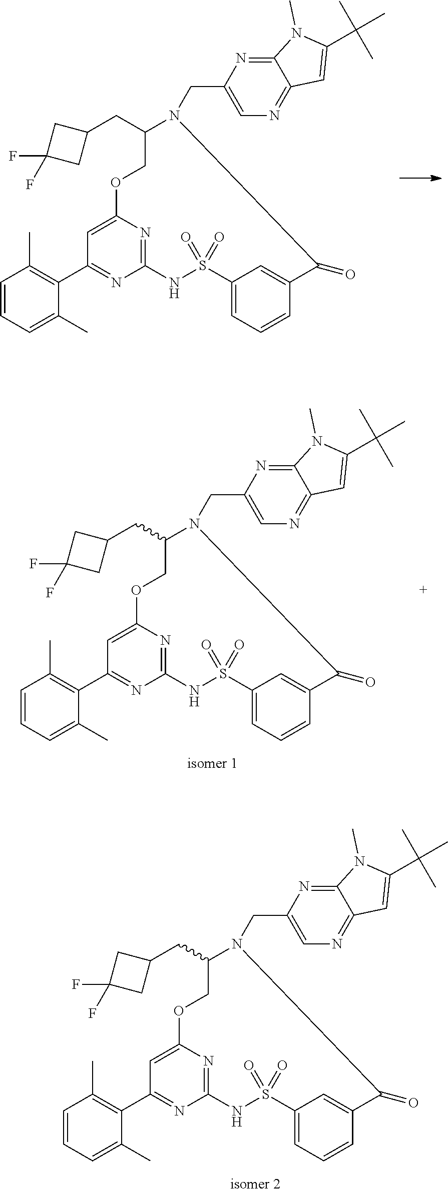 [SMILES: Cc1cccc(C)c1-c1cc2nc(n1)NS(=O)(=O)c1cccc(c1)C(=O)N(Cc1cnc3cc(C(C)(C)C)n(C)c3n1)C(CC1CC(F)(F)C1)CO2 ; Cc1cccc(C)c1-c1cc2nc(n1)NS(=O)(=O)c1cccc(c1)C(=O)N(Cc1cnc3cc(C(C)(C)C)n(C)c3n1)C(CC1CC(F)(F)C1)CO2 ; Cc1cccc(C)c1-c1cc2nc(n1)NS(=O)(=O)c1cccc(c1)C(=O)N(Cc1cnc3cc(C(C)(C)C)n(C)c3n1)C(CC1CC(F)(F)C1)CO2]

The enantiomers of 12-[(6-tert-butyl-5-methyl-pyrrolo[2,3-b]pyrazin-3-yl)methyl]-11-[(3,3-difluorocyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (24.4 mg, 0.03343 mmol) were separated by chiral SFC using a ChiralPak IC column (21.2×250 mm, 5 μm) at 40° C. Mobile phase was 48% MeOH (20 mM NH₃), 42% CO₂ at a 80 mL/min flow. Concentration of the sample was 15.3 mg/mL in methanol, injection volume 800 μL with an outlet pressure of 205 bar, detection wavelength of 210 nm. Each obtained enantiomer was purified by reverse phase HPLC (10-99% acetonitrile/5 mM aqueous HCl over 15 minutes). 12-[(6-tert-butyl-5-methyl-pyrrolo[2,3-b]pyrazin-3-yl)methyl]-11-[(3,3-difluorocyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (8.1 mg, 66%) was obtained as Peak 1, isomer 1 as a light yellow solid. ESI-MS m/z calc. 729.2909, found 730.6 (M+1)⁺; Retention time: 1.57 minutes. 12-[(6-tert-Butyl-5-methyl-pyrrolo[2,3-b]pyrazin-3-yl)methyl]-11-[(3,3-difluorocyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (8.4 mg, 67%) was obtained as Peak 2, isomer 2 as a light yellow solid. ESI-MS m/z calc. 729.2909, found 730.6 (M+1)⁺; Retention time: 1.58 minutes. LC method A.

### Example 121: Preparation of Compound IV-60 and Compound IV-61

### Step 1: 2-Amino-2-(1-isopropylcyclopropyl)ethanol

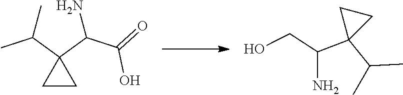

2-Amino-2-(1-isopropylcyclopropyl)acetic acid (hydrochloride salt) (700 mg, 3.614 mmol) was combined with THE (6 mL) in a nitrogen-purged flask with a nitrogen line. The flask was cooled to 0° C. in an ice bath, and BH₃ (15 mL of 1 M, 15.00 mmol) (in THF) was added by syringe. After 5 minutes the ice bath was removed, and the reaction mixture was allowed to warm to room temperature. After 20 minutes the nitrogen line was removed, and the reaction was stirred at room temperature for 18 hours. The reaction mixture was then cooled to 0° C. and quenched with the addition of 15 mL methanol. The reaction mixture was warmed to room temperature and stirred for 2 hours. Volatiles were removed by rotary evaporation, then additional 15 mL of methanol were added and removed by rotary evaporation. The resulting residue was diluted with 15 mL THE and evaporated 3×. The product was then suspended in DCM and acidified with HCl then evaporated a final time to give 2-amino-2-(1-isopropylcyclopropyl)ethanol (hydrochloride salt) (503 mg, 77%). ESI-MS m/z calc. 143.13101, found 144.2 (M+1)⁺; Retention time: 0.25 minutes; LC method B.

### Step 2: 3-[[4-[2-Amino-2-(1-isopropylcyclopropyl)ethoxy]-6-(2,6-dimethylphenyl)pyrimidin-2-yl]sulfamoyl]benzoic acid

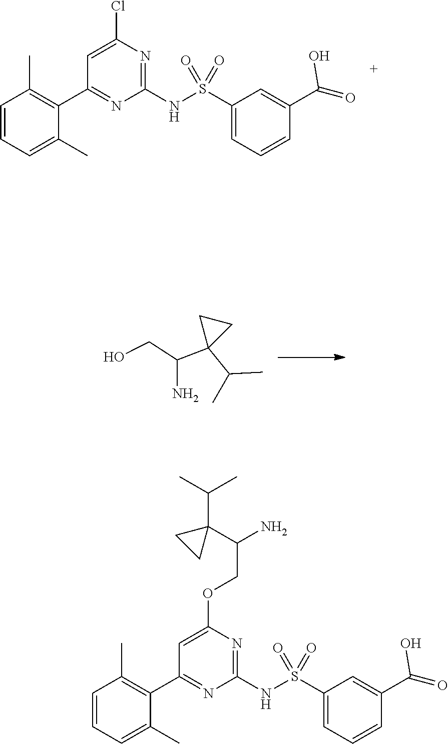

3-[[4-Chloro-6-(2,6-dimethylphenyl)pyrimidin-2-yl]sulfamoyl]benzoic acid (500 mg, 1.197 mmol) was combined with 2-amino-2-(1-isopropylcyclopropyl)ethanol (hydrochloride salt) (240 mg, 1.336 mmol) in anhydrous THE (3 mL) and stirred at room temperature until the reaction mixture became a homogeneous suspension (5 minutes). NaOtBu (700 mg, 7.284 mmol) was added in one portion and the reaction mixture was stirred for 25 minutes at room temperature. The reaction was then heated to 40° C. for an additional 25 minutes. The reaction was allowed to cool to room temperature and the reaction mixture was partitioned between 30 mL ethyl acetate and 30 mL 1 M HCl. The layers were separated and the aqueous was extracted an additional 3×20 mL ethyl acetate. The aqueous was then diluted with 30 mL brine and extracted an additional 2×20 mL ethyl acetate. The combined organics were washed with brine, dried over sodium sulfate, and concentrated to give 3-[[4-[2-amino-2-(1-isopropylcyclopropyl)ethoxy]-6-(2,6-dimethylphenyl)pyrimidin-2-yl]sulfamoyl]benzoic acid (hydrochloride salt) (662 mg, 97%) ESI-MS m/z calc. 524.20935, found 525.5 (M+1)⁺; Retention time: 0.46 minutes. LC method B.

### Step 3: 3-[[4-[2-[(6-Cyclopropylfuro[2,3-b]pyrazin-2-yl)methylamino]-2-(1-isopropylcyclopropyl)ethoxy]-6-(2,6-dimethylphenyl)pyrimidin-2-yl]sulfamoyl]benzoic acid

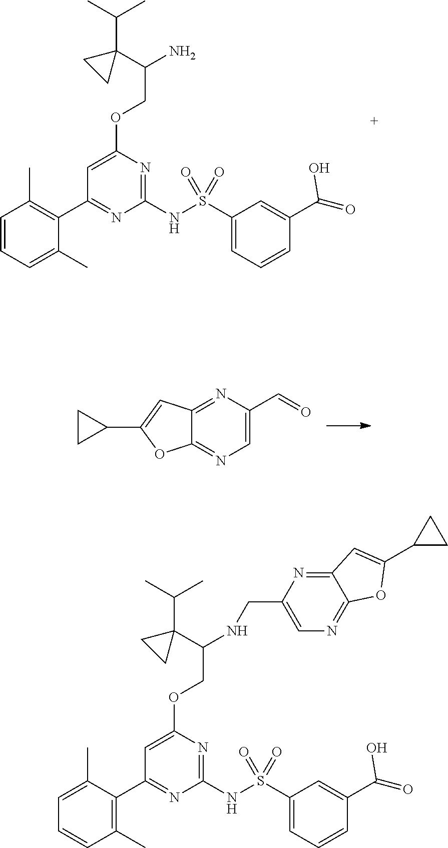 [SMILES: Cc1cccc(C)c1-c1cc(OCC(N)C2(C(C)C)CC2)nc(NS(=O)(=O)c2cccc(C(=O)O)c2)n1 ; O=Cc1cnc2oc(C3CC3)cc2n1 ; Cc1cccc(C)c1-c1cc(OCC(NCc2cnc3oc(C4CC4)cc3n2)C2(C(C)C)CC2)nc(NS(=O)(=O)c2cccc(C(=O)O)c2)n1]

3-[[4-[2-Amino-2-(1-isopropylcyclopropyl)ethoxy]-6-(2,6-dimethylphenyl)pyrimidin-2-yl]sulfamoyl]benzoic acid (hydrochloride salt) (60 mg, 0.1069 mmol) was combined with 6-cyclopropylfuro[2,3-b]pyrazine-2-carbaldehyde (approximately 22.13 mg, 0.1176 mmol) in DCM (0.4 mL). AcOH (approximately 11.55 mg, 10.94 μL, 0.1924 mmol) was added and the reaction mixture was stirred for 10 minutes at room temperature. The reaction was then cooled to 0° C. in an ice water bath and DIPEA (approximately 33.16 mg, 44.69 μL, 0.2566 mmol). After 15 minutes at 0° C. sodium triacetoxyborohydride (approximately 135.9 mg, 0.6414 mmol) was added and the reaction mixture was stirred for 2 hours at 0° C. The reaction mixture was then quenched with 3M HCl (aqueous) diluted with methanol and DMSO until it became homogeneous, then filtered and purified by preparative HPLC (1-99% ACN in water, HCl modifier, 15 minute run) to give 3-[[4-[2-[(6-cyclopropylfuro[2,3-b]pyrazin-2-yl)methylamino]-2-(1-isopropylcyclopropyl)ethoxy]-6-(2,6-dimethylphenyl)pyrimidin-2-yl]sulfamoyl]benzoic acid (hydrochloride salt) (45.1 mg, 58%). ESI-MS m/z calc. 696.273, found 697.7 (M+1)⁺; Retention time: 0.55 minutes; LC method B.

### Step 4: 12-[(6-Cyclopropylfuro[2,3-b]pyrazin-2-yl)methyl]-6-(2,6-dimethylphenyl)-11-(1-isopropylcyclopropyl)-2,2-dioxo-9-oxa-2λ⁶-thia-3,5,12,19-tetrazatricyclo[12.3.1.14,8]nonadeca-1(18),4(19),5,7,14,16-hexaen-13-one

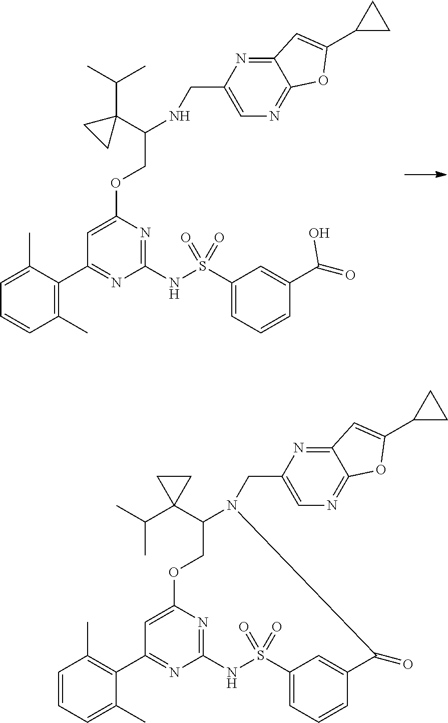 [SMILES: Cc1cccc(C)c1-c1cc(OCC(NCc2cnc3oc(C4CC4)cc3n2)C2(C(C)C)CC2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc3oc(C4CC4)cc3n1)C(C1(C(C)C)CC1)CO2]

3-[[4-[2-[(6-cyclopropylfuro[2,3-b]pyrazin-2-yl)methylamino]-2-(1-isopropylcyclopropyl)ethoxy]-6-(2,6-dimethylphenyl)pyrimidin-2-yl]sulfamoyl]benzoic acid (hydrochloride salt) (45.1 mg, 0.06150 mmol) was combined with CDMT (17 mg, 0.09683 mmol) in DMF. N-methylmorpholine (50 μL, 0.4548 mmol) was added dropwise and the reaction mixture was stirred at room temperature for the indicated time. The reaction was then evaporated under reduced pressure and the resulting residue was purified by chromatography on silica gel, eluting with a 0-15% methanol in DCM gradient to give 12-[(6-cyclopropylfuro[2,3-b]pyrazin-2-yl)methyl]-6-(2,6-dimethylphenyl)-11-(1-isopropylcyclopropyl)-2,2-dioxo-9-oxa-2λ⁶-thia-3,5,12,19-tetrazatricyclo[12.3.1.14,8]nonadeca-1(18),4(19),5,7,14,16-hexaen-13-one (17.3 mg, 41%). ESI-MS m/z calc. 678.26245, found 679.6 (M+1)⁺; Retention time: 0.79 minutes; LC method B.

### Step 5: 12-[(6-Cyclopropylfuro[2,3-b]pyrazin-2-yl)methyl]-6-(2,6-dimethylphenyl)-11-(1-isopropylcyclopropyl)-2,2-dioxo-9-oxa-2λ⁶-thia-3,5,12,19-tetrazatricyclo[12.3.1.14,8]nonadeca-1(18),4(19),5,7,14,16-hexaen-13-one, isomer 1 (Compound IV-60), and 12-[(6-cyclopropylfuro[2,3-b]pyrazin-2-yl)methyl]-6-(2,6-dimethylphenyl)-11-(1-isopropyl-cyclopropyl)-2,2-dioxo-9-oxa-2λ⁶-thia-3,5,12,19-tetrazatricyclo[12.3.1.14,8]nonadeca-1(18),4(19),5,7,14,16-hexaen-13-one, isomer 2 (Compound IV-61

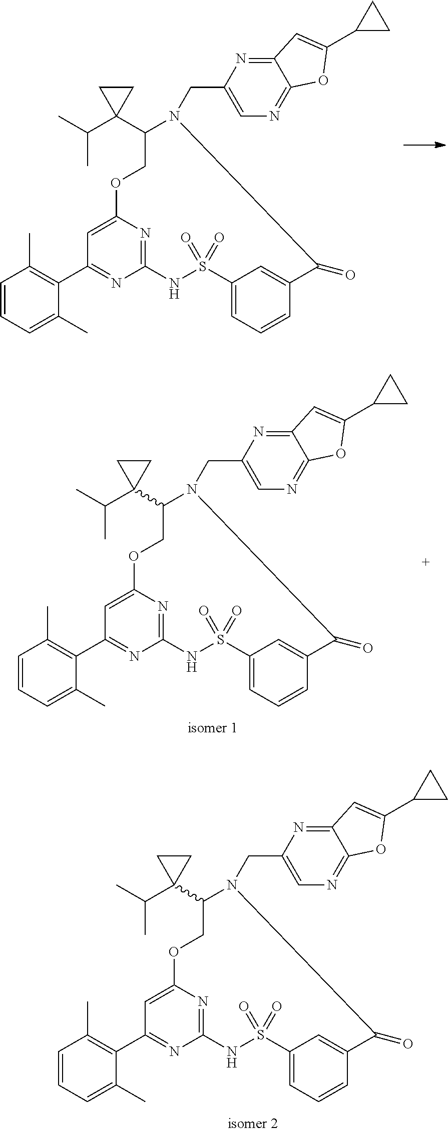 [SMILES: Cc1cccc(C)c1-c1cc2nc(n1)NS(=O)(=O)c1cccc(c1)C(=O)N(Cc1cnc3oc(C4CC4)cc3n1)C(C1(C(C)C)CC1)CO2 ; Cc1cccc(C)c1-c1cc2nc(n1)NS(=O)(=O)c1cccc(c1)C(=O)N(Cc1cnc3oc(C4CC4)cc3n1)C(C1(C(C)C)CC1)CO2 ; Cc1cccc(C)c1-c1cc2nc(n1)NS(=O)(=O)c1cccc(c1)C(=O)N(Cc1cnc3oc(C4CC4)cc3n1)C(C1(C(C)C)CC1)CO2]

The enantiomers of 12-[(6-cyclopropylfuro[2,3-b]pyrazin-2-yl)methyl]-6-(2,6-dimethylphenyl)-11-(1-isopropylcyclopropyl)-2,2-dioxo-9-oxa-2λ⁶-thia-3,5,12,19-tetrazatricyclo[12.3.1.14,8]nonadeca-1(18),4(19),5,7,14,16-hexaen-13-one (17.3 mg, 0.02549 mmol) were separated by chiral SFC using a ChiralPak AS (21.2×250 mm, 5 μM) column at 40° C. Mobile phase was 38% MeOH (20 mM NH₃), 62% CO₂ at 70 mL/min flow. Concentration of the sample was 10.8 mg/mL in methanol (no modifier), injection volume was 800 μL, outlet pressure at 171 bar, and detection wavelength 210 nm. The first eluting enantiomer was collected and further purified by preparative HPLC (1-99% ACN in water, HCl modifier, 15 minute run) to give Enantiomer 1 (Peak 1 SFC), 12-[(6-cyclopropylfuro[2,3-b]pyrazin-2-yl)methyl]-6-(2,6-dimethylphenyl)-11-(1-isopropylcyclopropyl)-2,2-dioxo-9-oxa-2λ⁶-thia-3,5,12,19-tetrazatricyclo[12.3.1.14,8]nonadeca-1(18),4(19),5,7,14,16-hexaen-13-one (4.6 mg, 25%) ¹H NMR (400 MHz, CDCl₃) δ 8.75 (t, J=1.9 Hz, 1H), 8.32 (s, 1H), 8.10 (d, J=8.0 Hz, 1H), 7.87 (dt, J=7.6, 1.4 Hz, 1H), 7.65 (t, J=7.8 Hz, 1H), 7.20 (t, J=7.6 Hz, 1H), 7.06 (d, J=7.6 Hz, 2H), 6.63 (s, 1H), 6.20 (s, 1H), 5.42-5.29 (m, 2H), 4.58 (d, J=15.0 Hz, 1H), 4.40 (d, J=7.6 Hz, 2H), 2.13 (ddd, J=13.6, 8.2, 5.2 Hz, 1H), 2.03 (s, 6H), 1.87 (p, J=6.8 Hz, 1H), 1.15 (ddt, J=11.2, 8.4, 2.9 Hz, 4H), 0.95 (dt, J=10.6, 6.1 Hz, 1H), 0.83 (dt, J=10.4, 5.4 Hz, 1H), 0.71-0.52 (m, 7H), 0.18-0.09 (m, 1H). ESI-MS m/z calc. 678.26245, found 679.7 (M+1)⁺; Retention time: 1.99 minutes. and Enantiomer 2 (Peak 2 SFC), 12-[(6-cyclopropyl furo[2,3-b]pyrazin-2-yl)methyl]-6-(2,6-dimethylphenyl)-11-(1-isopropylcyclopropyl)-2,2-dioxo-9-oxa-2λ⁶-thia-3,5,12,19-tetrazatricyclo[12.3.1.14,8]nonadeca-1(18),4(19),5,7,14,16-hexaen-13-one (4.6 mg, 26%) ¹H NMR (400 MHz, CDCl₃) δ 8.74 (d, J=1.8 Hz, 1H), 8.32 (s, 1H), 8.11 (d, J=7.9 Hz, 1H), 7.88 (d, J=7.6 Hz, 1H), 7.66 (t, J=7.8 Hz, 1H), 7.21 (t, J=7.6 Hz, 1H), 7.07 (d, J=7.6 Hz, 2H), 6.63 (s, 1H), 6.21 (s, 1H), 5.45-5.37 (m, 1H), 5.33 (d, J=15.1 Hz, 1H), 4.57 (d, J=15.0 Hz, 1H), 4.46-4.34 (m, 2H), 2.18-2.08 (m, 1H), 2.05 (s, 6H), 1.90-1.83 (m, 1H), 1.20-1.10 (m, 4H), 0.95 (dt, J=10.5, 5.9 Hz, 1H), 0.84 (dt, J=10.6, 5.5 Hz, 1H), 0.68-0.52 (m, 7H), 0.18-0.10 (m, 1H). ESI-MS m/z calc. 678.26245, found 679.7 (M+1)⁺; Retention time: 1.98 minutes. LC method A.

### Example 122: Preparation of Compound IV-63

### Step 1: (2-Isopropylimidazo[1,2-a]pyrimidin-7-yl)methanol

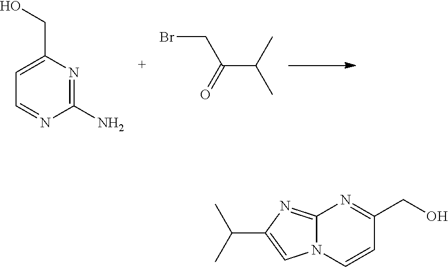

To a solution of (2-aminopyrimidin-4-yl)methanol (1 g, 7.9918 mmol) in DMF (24 mL) was added 1-bromo-3-methyl-butan-2-one (1.4420 g, 1.03 mL, 8.7379 mmol) and the reaction mixture was stirred at room temperature for 2 days. Then solvent was removed under reduced pressure and the crude product was diluted in ethyl acetate (20 mL) and a saturated aqueous solution of sodium bicarbonate (15 mL) was added. The organic layer was separated, and the aqueous solution was extracted three times using ethyl acetate (20 mL×3). The combined organic layers were washed using brine (20 mL) dried over sodium sulfate, filtered, and concentrated under reduced pressure. The crude product was then triturated in diethyl ether (20 mL) to afford (2-isopropylimidazo[1,2-a]pyrimidin-7-yl)methanol (760 mg, 47%) as a yellowish solid. ¹H NMR (400 MHz, CD₃OD) δ 8.72 (d, J=6.8 Hz, 1H), 7.53 (s, 1H), 7.14 (d, J=6.8 Hz, 1H), 4.70 (s, 2H), 3.16-3.00 (m, 1H), 1.35 (d, J=6.8 Hz, 6H). One exchangeable proton is missing. ESI-MS m/z calc. 191.1059, found 192.2 (M+1)⁺; Retention time: 2.19 minutes. LC method K.

### Step 2: 2-Isopropylimidazo[1,2-a]pyrimidine-7-carbaldehyde

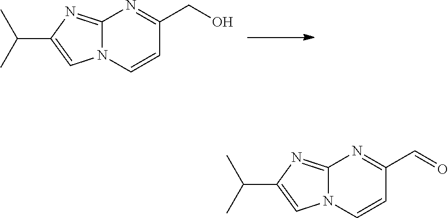

To a solution of (2-isopropylimidazo[1,2-a]pyrimidin-7-yl)methanol (760 mg, 3.7756 mmol) in DCM (25 mL) was added MnO₂ (3.45 g, 39.684 mmol). The solution was stirred at room temperature for 3 days then the reaction mixture was filtered through a celite pad. The filtrate was concentrated under vacuum to afford 2-isopropylimidazo[1,2-a]pyrimidine-7-carbaldehyde (281.6 mg, 38%) as a pale yellowish powder. ¹H NMR (400 MHz, CDCl₃) δ 10.02 (s, 1H), 8.49 (d, J=7.0 Hz, 1H), 7.53 (s, 1H), 7.47 (d, J=7.0 Hz, 1H), 3.31-3.20 (m, 1H), 1.43 (d, J=7.0 Hz, 6H). ESI-MS m/z calc. 189.0902, found 190.1 (M+1)⁺; Retention time: 2.32 minutes. LC method K.

### Step 3: 3-[[4-[(2R)-3-(1-Bicyclo[1.1.1]]pentanyl)-2-[(2-isopropylimidazo[1,2-a]pyrimidin-7-yl)methylamino]propoxy]-6-(2,6-dimethylphenyl)pyrimidin-2-yl]sulfamoyl]benzoic acid

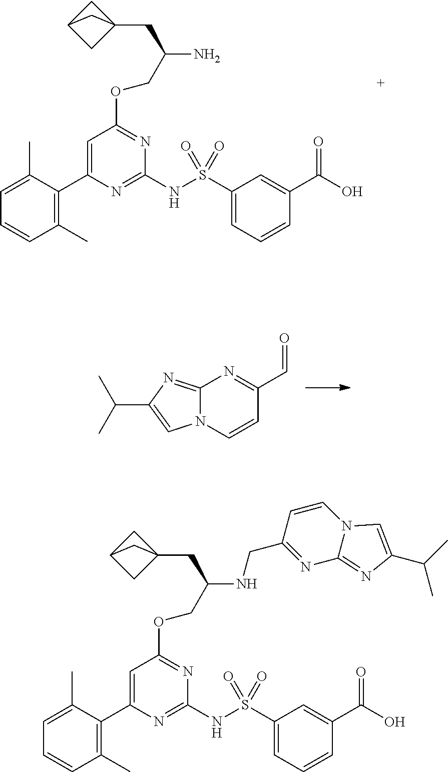 [SMILES: Cc1cccc(C)c1-c1cc(OC[C@H](N)CC23CC(C2)C3)nc(NS(=O)(=O)c2cccc(C(=O)O)c2)n1 ; CC(C)c1cn2ccc(C=O)nc2n1 ; Cc1cccc(C)c1-c1cc(OC[C@@H](CC23CC(C2)C3)NCc2ccn3cc(C(C)C)nc3n2)nc(NS(=O)(=O)c2cccc(C(=O)O)c2)n1]

In a 4 mL vial, to a stirred mixture of 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (50 mg, 0.08049 mmol) and 2-isopropylimidazo[1,2-a]pyrimidine-7-carbaldehyde (15.3 mg, 0.08086 mmol) in anhydrous dichloromethane (350 μL) were added glacial acetic acid (15 μL, 0.2638 mmol) and DIPEA (40 μL, 0.2296 mmol), in that order, at 0° C. under nitrogen. After 2 min, sodium triacetoxyborohydride (64 mg, 0.3020 mmol) was added to the yellow solution. The heterogeneous reaction was stirred at that temperature for 15 min. Then the reaction was quenched with aqueous 1 M HCl (0.5 mL), MeOH (0.5 mL) and DMSO (0.5 mL) and purified by preparative reverse-phase HPLC [1-99% acetonitrile in water (containing 5 mM HCl) over 15 min] to furnish product as a yellow solid. 3-[[4-[(2R)-3-(1-bicyclo[1.1.1]pentanyl)-2-[(2-isopropylimidazo[1,2-a]pyrimidin-7-yl)methylamino]propoxy]-6-(2,6-dimethylphenyl)pyrimidin-2-yl]sulfamoyl]benzoic acid (Dihydrochloride salt) (41 mg, 66%) ESI-MS m/z calc. 695.289, found 696.2 (M+1)⁺; Retention time: 1.04 minutes. LC method A.

### Step 4: (11R)-11-(1-Bicyclo[1.1.1]pentanylmethyl)-6-(2,6-dimethylphenyl)-12-[(2-isopropyl-imidazo[1,2-a]pyrimidin-7-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-63

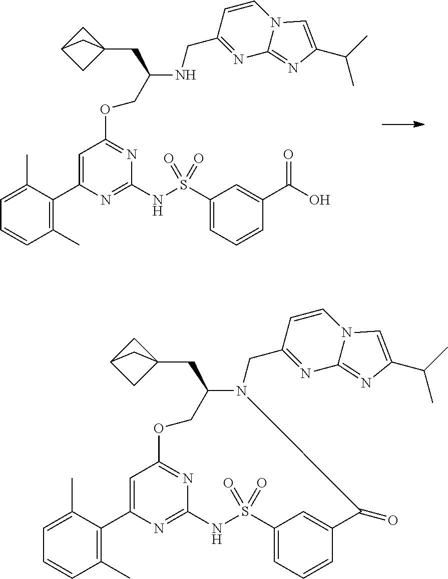 [SMILES: Cc1cccc(C)c1-c1cc(OC[C@@H](CC23CC(C2)C3)NCc2ccn3cc(C(C)C)nc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccn3cc(C(C)C)nc3n1)[C@H](CC13CC(C1)C3)CO2]

To a stirred solution of 3-[[4-[(2R)-3-(1-bicyclo[1.1.1]pentanyl)-2-[(2-isopropylimidazo[1,2-a]pyrimidin-7-yl)methylamino]propoxy]-6-(2,6-dimethylphenyl)pyrimidin-2-yl]sulfamoyl]benzoic acid (dihydrochloride salt) (41 mg, 0.05333 mmol) in anhydrous DMF (2.0 mL) was added 2-chloro-4,6-dimethoxy-1,3,5-triazine (13 mg, 0.07404 mmol) (CDMT), followed by addition of 4-methylmorpholine (50 μL, 0.4548 mmol) at 0-4° C. (ice-water bath) under nitrogen. The yellow reaction was allowed to stir at that temperature for 5 min, then allowed to stir at room temperature for 3 h. Purification of the reaction mixture by preparative reverse-phase HPLC (1-99% acetonitrile in water over 15 min, 5 mM HCl as modifier) furnished product as a white solid. (11R)-11-(1-bicyclo[1.1.1]pentanyl methyl)-6-(2,6-dimethylphenyl)-12-[(2-isopropylimidazo[1,2-a]pyrimidin-7-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (25.5 mg, 66%) ¹H NMR (400 MHz, MeOD) δ 9.08 (d, J=6.9 Hz, 1H), 8.86 (t, J=1.8 Hz, 1H), 8.09 (dt, J=7.9, 1.5 Hz, 1H), 7.95-7.91 (m, 1H), 7.85-7.78 (m, 1H), 7.73 (dd, J=9.1, 7.3 Hz, 2H), 7.29 (t, J=7.6 Hz, 1H), 7.16 (d, J=7.6 Hz, 2H), 6.31 (s, 1H), 5.68 (dd, J=10.7, 3.8 Hz, 1H), 5.19 (d, J=17.4 Hz, 1H), 4.81 (d, J=17.5 Hz, 1H), 4.24 (t, J=11.1 Hz, 1H), 4.14 (tt, J=11.3, 3.4 Hz, 1H), 3.24 (hept, J=6.9 Hz, 1H), 2.43 (s, 1H), 2.14 (s, 6H), 2.04 (dd, J=15.7, 10.5 Hz, 1H), 1.84 (dd, J=15.7, 3.0 Hz, 1H), 1.58 (s, 6H), 1.44 (d, J=7.0 Hz, 6H). ESI-MS m/z calc. 677.27844, found 678.2 (M+1)⁺; Retention time: 1.33 minutes. LC method A.

### Example 123: Preparation of Compound IV-70

### Step 1: (2S)-2-Amino-3-(trifluoromethoxy)propan-1-ol

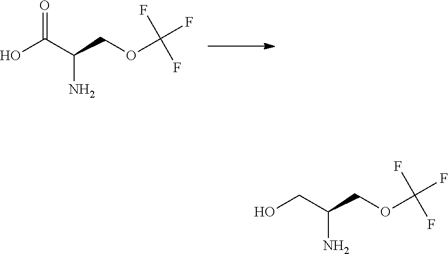 [SMILES: N[C@H](COC(F)(F)F)C(=O)O ; N[C@@H](CO)COC(F)(F)F]

(2R)-2-Amino-3-(trifluoromethoxy)propanoic acid (hydrochloride salt) (250 mg, 1.193 mmol) was combined with borane:tetrahydrofuran (4 mL of 1 M, 4.000 mmol) in THE (5 mL) at 0° C. in a nitrogen-purged vial. The reaction was allowed to warm to room temperature after 10 minutes and was stirred for 72 hours. The reaction was cooled to 0° C., quenched with 20 mL methanol and after 20 minutes warmed to room temperature. After 2 hours, volatiles were removed by rotary evaporation, 20 mL methanol were again added and evaporated, followed by addition of 20 mL THE and evaporation 2×. The resulting product was dissolved in DCM and HCl (900 μL of 4 M, 3.600 mmol) in dioxane was added and volatiles were removed to give (2S)-2-amino-3-(trifluoromethoxy)propan-1-ol (hydrochloride salt) (185 mg, 79%).

### Step 2: 3-{[(]P)-4-[(2S)-2-Amino-3-(trifluoromethoxy)propoxy]-5-methyl-6-[2-methyl-6-(2-methylpropyl)phenyl]pyrimidin-2-yl]sulfamoyl}benzoic acid

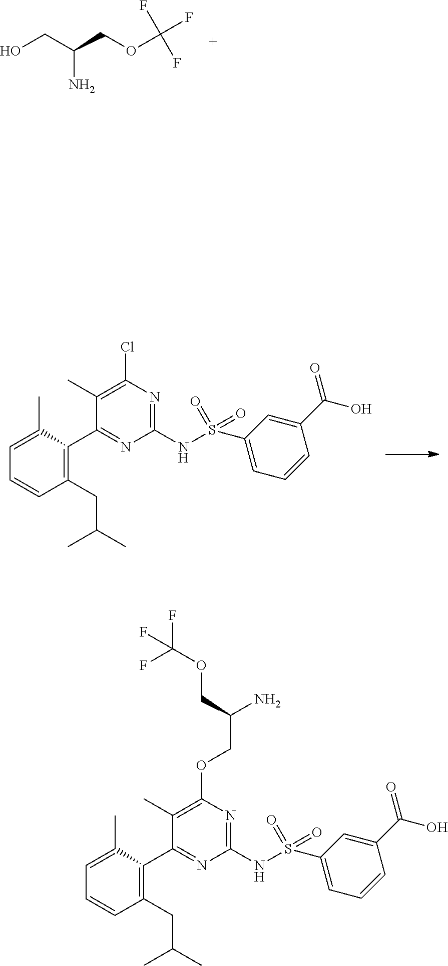

(2S)-2-Amino-3-(trifluoromethoxy)propan-1-ol (hydrochloride salt) (100 mg, 0.5113 mmol) was combined with (5P)-3-[[4-Chloro-6-(2-isobutyl-6-methyl-phenyl)-5-methyl-pyrimidin-2-yl]sulfamoyl]benzoic acid (185 mg, 0.3903 mmol) in a anhydrous THE (900 μL). The reaction was stirred at room temperature for 15 minutes, then Sodium tert-butoxide (250 mg, 2.601 mmol) was added in one portion. The reaction vial became warm to the touch and was stirred without any additional external heating for 45 minutes. The reaction mixture was then acidified with 1 mL 4M HCl in dioxane, diluted with DMSO, filtered, and purified by preparative HPLC (1-99% ACN in water, HCl modifier, 15 minute run) to give 3-{[(1P)-4-[(2S)-2-amino-3-(trifluoromethoxy)propoxy]-5-methyl-6-[2-methyl-6-(2-methylpropyl)phenyl]pyrimidin-2-yl]sulfamoyl}benzoic acid (hydrochloride salt) (82.0 mg, 33%) ESI-MS m/z calc. 596.19165, found 597.2 (M+1)⁺; Retention time: 0.52 minutes. LC method B.

### Step 3: 3-{[(5P,5P)-4-[(2S)-2-[({6-tert-Butyl-5-methyl-5H-pyrrolo[2,3-b]pyrazin-3-yl}methyl)amino]-3-(trifluoromethoxy)propoxy]-5-methyl-6-[2-methyl-6-(2-methylpropyl)phenyl]pyrimidin-2-yl]sulfamoyl}benzoic acid

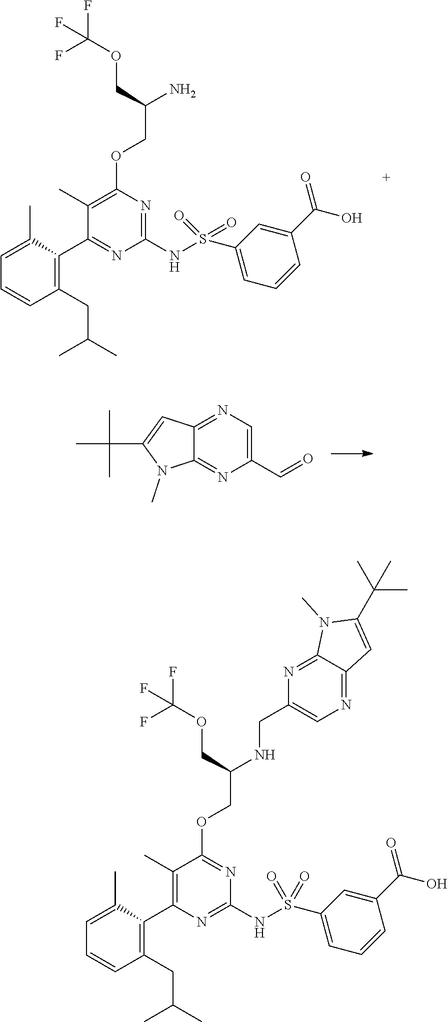

3-{[(1P)-4-[(2S)-2-Amino-3-(trifluoromethoxy)propoxy]-5-methyl-6-[2-methyl-6-(2-methylpropyl)phenyl]pyrimidin-2-yl]sulfamoyl}benzoic acid (hydrochloride salt) (18 mg, 0.02843 mmol) was combined with 6-tert-butyl-5-methyl-pyrrolo[2,3-b]pyrazine-3-carbaldehyde (7 mg, 0.03222 mmol) in DCM (0.4 mL). After stirring for 5 minutes at room temperature AcOH (5 μL, 0.08792 mmol) was added, and stirring was continued at room temperature for 15 minutes. The reaction mixture was then cooled to 0° C. in an ice bath and DIPEA (20 μL, 0.1148 mmol) was added. After 15 minutes at 0° C. sodium triacetoxyborohydride (45 mg, 0.2123 mmol) was added in one portion. After stirring for 90 minutes at 0° C. the reaction was quenched with 0.3 mL 1 M HCl, then diluted with methanol until it became homogeneous. The reaction mixture was filtered and purified by preparative HPLC (1-99% ACN in water, HCl modifier, 15 minutes run) to give 3-{[(5P,5P)-4-[(2S)-2-[({6-tert-butyl-5-methyl-5H-pyrrolo[2,3-b]pyrazin-3-yl}methyl)amino]-3-(trifluoromethoxy)propoxy]-5-methyl-6-[2-methyl-6-(2-methylpropyl)phenyl]pyrimidin-2-yl]sulfamoyl}benzoic acid (hydrochloride salt) (11.1 mg, 47%). ESI-MS m/z calc. 797.31824, found 798.5 (M+1)⁺; Retention time: 0.66 minutes; LC method B.

### Step 4: (6P,11S)-12-({6-tert-Butyl-5-methyl-5H-pyrrolo[2,3-b]pyrazin-3-yl}methyl)-7-methyl-6-[2-methyl-6-(2-methylpropyl)phenyl]-11-[(trifluoromethoxy)methyl]-9-oxa-226-thia-3,5,12,19-tetraazatricyclo[12.3.1.1∧{4,8}]nonadeca-1(17),4,6,8(19),14(18),15-hexaene-2,2,13-trione (Compound IV-70

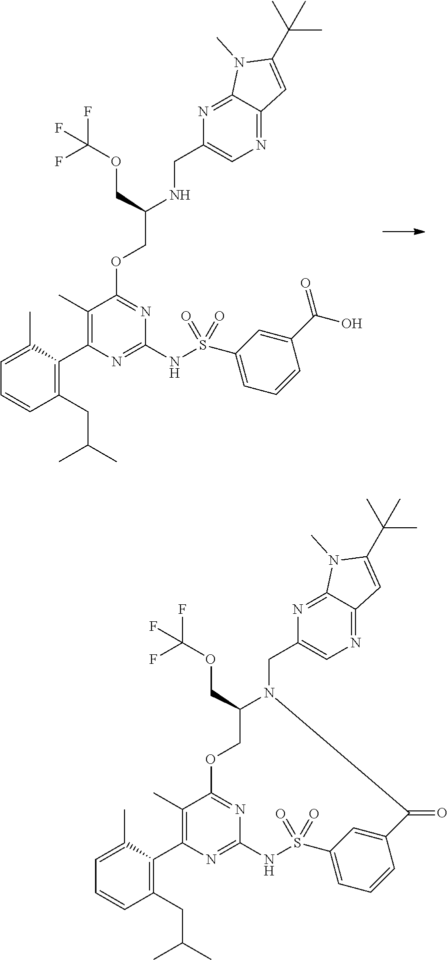

3-{[(5P,5P)-4-[(2S)-2-[({6-tert-Butyl-5-methyl-5H-pyrrolo[2,3-b]pyrazin-3-yl}methyl)amino]-3-(trifluoromethoxy)propoxy]-5-methyl-6-[2-methyl-6-(2-methylpropyl)phenyl]pyrimidin-2-yl]sulfamoyl}benzoic acid (hydrochloride salt) (11.1 mg, 0.01330 mmol) was combined with CDMT (3.5 mg, 0.01994 mmol) in DMF (1.5 mL). N-methylmorpholine (10 μL, 0.09095 mmol) was added, and the reaction mixture was stirred at room temperature for 18 hours. The reaction mixture was concentrated by rotary evaporation, and after removal of the volatiles the resulting residue was dissolved in 1:1 DMSO/methanol, filtered, and purified by preparative HPLC (1-99% ACN in water, HCl modifier, 15 minute run) to give (6P,11S)-12-({6-tert-butyl-5-methyl-5H-pyrrolo[2,3-b]pyrazin-3-yl}methyl)-7-methyl-6-[2-methyl-6-(2-methylpropyl)phenyl]-11-[(trifluoromethoxy)methyl]-9-oxa-2λ⁶-thia-3,5,12,19-tetraazatricyclo[12.3.1.1∧{4,8}]nonadeca-1(17),4,6,8(19),14(18),15-hexaene-2,2,13-trione (3.8 mg, 36%). ESI-MS m/z calc. 779.3077, found 780.5 (M+1)⁺; Retention time: 2.12 minutes; LC method A. ¹H NMR (400 MHz, CDCl₃) δ 8.92 (s, 1H), 8.53 (s, 1H), 8.11 (d, J=7.9 Hz, 1H), 7.89 (d, J=7.6 Hz, 1H), 7.69 (t, J=7.8 Hz, 1H), 7.28 (s, 1H), 7.10 (dd, J=15.2, 7.6 Hz, 2H), 6.68 (s, 1H), 5.66-5.59 (m, 1H), 5.55 (d, J=15.7 Hz, 1H), 4.63 (s, 1H), 4.52 (d, J=15.8 Hz, 1H), 4.40 (t, J=11.6 Hz, 1H), 4.32 (t, J=9.5 Hz, 1H), 4.16 (s, 1H), 4.14 (s, 3H), 2.22-2.10 (m, 2H), 1.91 (s, 3H), 1.80-1.76 (m, 1H), 1.71 (s, 3H), 1.53 (s, 9H), 0.80 (dd, J=6.6, 1.8 Hz, 6H).

### Example 124: Preparation of Compound IV-76

### Step 1: 1-(2,2-Dibromovinyl)-1-(trifluoromethyl)cyclopropane

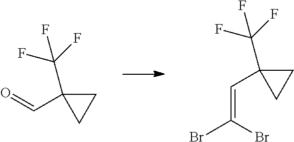 [SMILES: O=CC1(C(F)(F)F)CC1 ; FC(F)(F)C1(C=C(Br)Br)CC1]

To a solution of 1-(trifluoromethyl)cyclopropanecarbaldehyde (13.886 g, 100.56 mmol) in DCM was added CBr₄ (50 g, 150.77 mmol). The mixture was placed in a water bath and triphenylphosphine (80 g, 305.01 mmol) was added portionwise over 1 hour. The mixture was stirred at room temperature for 16 hours. The precipitate was filtered on a Celite pad and the Celite cake was washed with DCM (2×20 mL). The filtrate was carefully evaporated under reduced pressure and the product was triturated in pentanes (300 mL) for 4 hours. The precipitate was filtered on a silica pad and the silica cake was washed with pentane (3×50 mL). The solvent was carefully removed under reduced pressure to afford 1-(2,2-dibromovinyl)-1-(trifluoromethyl)cyclopropane (27.5 g, 70%) as a colorless liquid.

### Step 2: 1-Ethynyl-1-(trifluoromethyl)cyclopropane

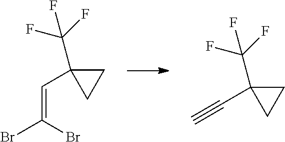

To a solution of 1-(2,2-dibromovinyl)-1-(trifluoromethyl)cyclopropane (27.5 g, 70.175 mmol) in anhydrous THF (200 mL) was added n-BuLi (solution in hexanes) (62 mL of 2.5 M, 155.00 mmol) dropwise over 30 minutes at −78° C. After addition, the reaction mixture was stirred at −78° C. for 1.5 h. Then it was quenched with ammonium chloride (7 mL), dried over magnesium sulfate and filtered on a pad of celite to give 1-ethynyl-1-(trifluoromethyl)cyclopropane as a solution in THF. The product was used as a solution for the next step.

### Step 3: 6-Chloro-3-[2-[1-(trifluoromethyl)cyclopropyl]ethynyl]pyrazin-2-amine

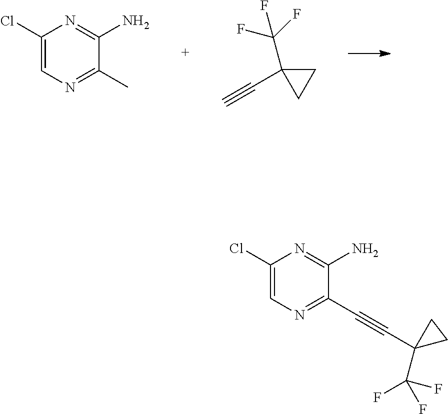

In a flask under nitrogen atmosphere containing a solution of 1-ethynyl-1-(trifluoromethyl)cyclopropane (9.4104 g, 70.175 mmol) in THE were added 3-bromo-6-chloro-pyrazin-2-amine (7.29 g, 34.973 mmol), triethylamine (11.616 g, 16 mL, 114.79 mmol), bis(triphenylphosphine)palladium(II) dichloride (2.53 g, 3.6045 mmol) and copper iodide (725 mg, 3.8068 mmol). The resulting mixture was stirred at room temperature under nitrogen atmosphere (sealed tube) for 16 hours. The reaction mixture was filtered on celite, and the cake was washed with acetonitrile (5×30 mL). The organics were concentrated under reduced pressure and the resulting residue was purified by silica gel chromatography (0-100% EtOAc in Heptanes) to afford 6-chloro-3-[2-[1-(trifluoromethyl)cyclopropyl]ethynyl]pyrazin-2-amine (8.80 g, 96%) as an orange solid. ESI-MS m/z calc. 261.0281, found 262.2 (M+1)⁺; Retention time: 1.8 minutes. LC method I.

### Step 4: 3-Chloro-6-[1-(trifluoromethyl)cyclopropyl]-5H-pyrrolo[2,3-b]pyrazine

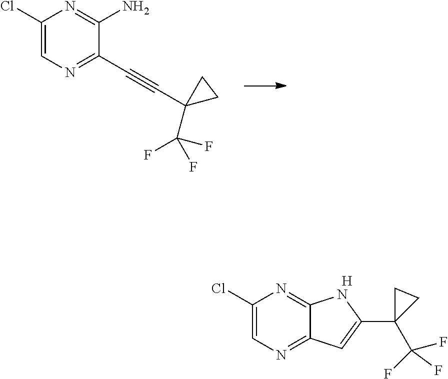

Potassium tert-butoxide (12 g, 106.94 mmol) was added to a solution of 6-chloro-3-[2-[1-(trifluoromethyl)cyclopropyl]ethynyl]pyrazin-2-amine (8.80 g, 33.602 mmol) in tert-butanol (170 mL) and heated at 70° C. for 16 h. The mixture was allowed to cool down to room temperature, then quenched with water (20 mL). The volatiles were removed under reduced pressure and saturated aqueous ammonium chloride solution (300 mL) was added. The mixture was cooled at 0° C. and stirred for 20 minutes. The resulting precipitate was filtered and washed with distilled water (2×50 mL). The precipitate was dissolved in EtOAc (200 mL), dried over anhydrous sodium sulfate, filtered and concentrated under reduced pressure to provide 3-chloro-6-[1-(trifluoromethyl)cyclopropyl]-5H-pyrrolo[2,3-b]pyrazine (8.74 g, 99%) as a brown solid. ESI-MS m/z calc. 261.0281, found 262.2 (M+1)⁺; Retention time: 1.74 minutes. This product was used in the next step without further purification. LC method I.

### Step 5: 3-Chloro-5-methyl-6-[1-(trifluoromethyl)cyclopropyl]pyrrolo[2,3-b]pyrazine

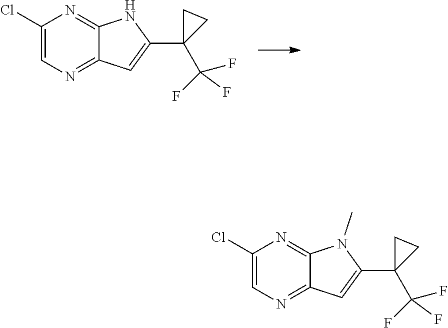 [SMILES: FC(F)(F)C1(c2cc3ncc(Cl)nc3[nH]2)CC1 ; Cn1c(C2(C(F)(F)F)CC2)cc2ncc(Cl)nc21]

Sodium hydride (60% w/w in oil) (1.18 g, 29.503 mmol) was added portionwise to a solution of 3-chloro-6-[1-(trifluoromethyl)cyclopropyl]-5H-pyrrolo[2,3-b]pyrazine (3.81 g, 14.242 mmol) in DMF (76 mL) at 0° C. The suspension was stirred at this temperature for 15 min. Dimethyl sulfate (2.7993 g, 2.1 mL, 22.193 mmol) was added and left stirring at 0° C. for 15 min. The reaction was then stirred at room temperature for 1 h. The reaction mixture was cooled back to 0° C. and quenched with saturated aqueous ammonium chloride solution (100 mL). The resulting precipitate was filtered, washed with pure water (2×25 mL) and a small volume of heptanes (5 mL) then dried under high vacuum to provide 3.65 g of the desired product as a brown solid. The filtrate was extracted with EtOAc (3×50 mL) and the combined organic layers were washed with brine (50 mL), dried over anhydrous sodium sulfate, filtered and concentrated under reduced pressure. The resulting precipitate was purified by reversed phase chromatography (Gradient: 5-100% MeCN in water) to provide 318 mg of the desired product as a dark yellow solid. The two product obtained were combined to afford 3-chloro-5-methyl-6-[1-(trifluoromethyl)cyclopropyl]pyrrolo[2,3-b]pyrazine (3.96 g, 101%) as a brown solid. ESI-MS m/z calc. 275.0437, found 276.2 (M+1)⁺; Retention time: 1.89 minutes. LC method I.

### Step 6: Methyl 5-methyl-6-[1-(trifluoromethyl)cyclopropyl]pyrrolo[2,3-b]pyrazine-3-carboxylate

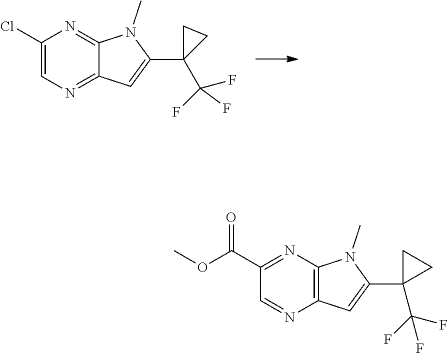 [SMILES: Cn1c(C2(C(F)(F)F)CC2)cc2ncc(Cl)nc21 ; COC(=O)c1cnc2cc(C3(C(F)(F)F)CC3)n(C)c2n1]

To a suspension of 3-chloro-5-methyl-6-[1-(trifluoromethyl)cyclopropyl]pyrrolo[2,3-b]pyrazine (3.96 g, 14.351 mmol) in anhydrous methanol (72 mL) was added triethylamine (4.5012 g, 6.2 mL, 44.483 mmol). Nitrogen was bubbled into the mixture for 15 minutes and then 1,1′-bis(diphenylphosphino)ferrocene palladium(II) chloride, complex with dichloromethane (592 mg, 0.7249 mmol) was added. Nitrogen bubbling was then continued for 5 minutes. The mixture was then stirred at 100° C. under 50 psi of carbon monoxide pressure for 18 hours. The mixture was then allowed to cool down to room temperature and was filtered on a Celite pad. The Celite cake was washed with EtOAc (2×30 mL) and concentrated under reduced pressure. Water (100 mL) and EtOAc (100 mL) were then added, and the mixture was extracted with EtOAc (3×100 mL). The combined organic layers were washed with brine (100 mL) and dried over anhydrous sodium sulfate, filtered and concentrated under reduced pressure. The resulting crude was purified by silica gel chromatography (Gradient: 0-60% EtOAc in heptane) to afford methyl 5-methyl-6-[1-(trifluoromethyl)cyclopropyl]pyrrolo[2,3-b]pyrazine-3-carboxylate (3.72 g, 85%) as a light yellow solid. ¹H NMR (400 MHz, CDCl₃) δ 9.24 (s, 1H), 6.86 (s, 1H), 4.06 (s, 3H), 4.03 (s, 3H), 1.69-1.64 (m, 2H), 1.32-1.27 (m, 2H). ¹⁹F NMR (377 MHz, CDCl₃) δ −68.55 (s, 3F). ESI-MS m/z calc. 299.0882, found 300.0 (M+1)⁺; Retention time: 3.96 minutes. LC method J.

### Step 7: [5-Methyl-6-[1-(trifluoromethyl)cyclopropyl]pyrrolo[2,3-b]pyrazin-3-yl]methanol

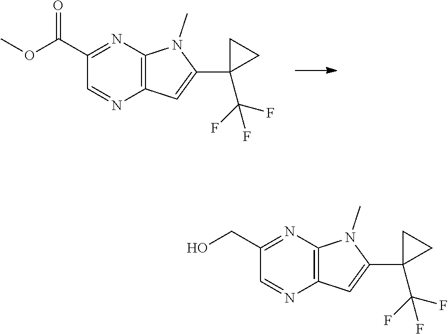 [SMILES: COC(=O)c1cnc2cc(C3(C(F)(F)F)CC3)n(C)c2n1 ; Cn1c(C2(C(F)(F)F)CC2)cc2ncc(CO)nc21]

To a stirred solution of methyl 5-methyl-6-[1-(trifluoromethyl)cyclopropyl]pyrrolo[2,3-b]pyrazine-3-carboxylate (1.1 g, 3.6722 mmol) in anhydrous DCM (40 mL) at −78° C. was added DIBAL (solution in toluene) (4 mL of 1 M, 4.0000 mmol) dropwise. The reaction was stirred at this temperature for 1 hour, then DIBAL (solution in toluene) (0.5 mL of 1 M, 0.5000 mmol) was added. After 1 hour, DIBAL (solution in toluene) (0.5 mL of 1 M, 0.5000 mmol) was added again. The reaction was stirred for 1 hour, then the temperature was allowed to warm to 0° C. A saturated solution of Rochelle salt (20 mL) was added, followed by EtOAc (20 mL) and the mixture was vigorously stirred at this temperature for 16 hours. The phases were separated, and the aqueous layer was extracted with EtOAc (3×15 mL). The combined organic layers were washed with brine (20 mL), dried over anhydrous sodium sulfate, filtered, and concentrated under reduced pressure. The product was dissolved in anhydrous DCM (40 mL). DIBAL (solution in toluene) (4.4 mL of 1 M, 4.4000 mmol) was added dropwise to the solution at −78° C. The reaction was stirred at this temperature for 1 hour, then DIBAL (solution in toluene) (1 mL of 1 M, 1.0000 mmol) was added. After 1 hour, the temperature was warmed to 0° C. A saturated solution of Rochelle salt (20 mL) was added, followed by EtOAc (20 mL) and the mixture was vigorously stirred at this temperature for 1 hour. The phases were separated, and the aqueous layer was extracted with EtOAc (3×20 mL). The combined organic layers were washed with brine (30 mL), dried over anhydrous sodium sulfate, filtered and concentrated under reduced pressure to afford [5-methyl-6-[1-(trifluoromethyl)cyclopropyl]pyrrolo[2,3-b]pyrazin-3-yl]methanol (925 mg, 92%) as a yellow solid. ¹H NMR (400 MHz, CDCl₃) δ 8.50 (s, 1H), 6.84 (s, 1H), 4.95 (s, 2H), 3.96 (s, 3H), 1.66-1.60 (m, 2H), 1.30-1.24 (m, 2H), (1H missing, labile proton). ¹⁹F NMR (377 MHz, CDCl₃) δ −68.67 (s, 3F). ESI-MS m/z calc. 271.0932, found 272.2 (M+1)⁺; Retention time: 1.55 minutes. LC method I.

### Step 8: 5-Methyl-6-[1-(trifluoromethyl)cyclopropyl]pyrrolo[2,3-b]pyrazine-3-carbaldehyde

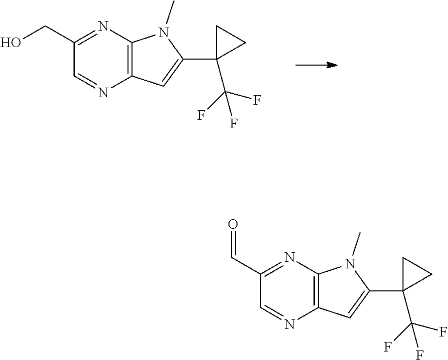 [SMILES: Cn1c(C2(C(F)(F)F)CC2)cc2ncc(CO)nc21 ; Cn1c(C2(C(F)(F)F)CC2)cc2ncc(C=O)nc21]

To a 0° C. solution of [5-methyl-6-[1-(trifluoromethyl)cyclopropyl]pyrrolo[2,3-b]pyrazin-3-yl]methanol (925 mg, 3.3762 mmol) in dry DCM (24 mL) was added Dess-Martin periodinane (1.8 g, 4.2439 mmol). The solution was stirred at room temperature for 1 hour. Dess-Martin periodinane (500 mg, 1.1789 mmol) was added and the mixture was stirred for 1 hour. A mixture of aqueous saturated solution of sodium thiosulfate (30 mL), saturated sodium bicarbonate (30 mL), water (25 mL) and an aqueous solution of NaOH 1M (10 mL, to reach pH=9) were added and the reaction mixture was stirred for 10 minutes. The phases were separated and the aqueous one was extracted with DCM (3×20 mL). The combined organic layers were washed with brine (30 mL), dried over anhydrous sodium sulfate, filtered, and concentrated under reduced pressure. The crude material was purified by silica gel chromatography (Gradient: 0-50% EtOAc in heptane) to afford 5-methyl-6-[1-(trifluoromethyl)cyclopropyl]pyrrolo[2,3-b]pyrazine-3-carbaldehyde (791 mg, 86%) as a light yellow solid. ¹H NMR (400 MHz, CDCl₃) δ 10.20 (s, 1H), 9.12 (s, 1H), 6.90 (s, 1H), 4.04 (s, 3H), 1.71-1.66 (m, 2H), 1.35-1.29 (m, 2H). ¹⁹F NMR (377 MHz, CDCl₃) δ −68.46 (s, 3F). ESI-MS m/z calc. 269.0776, found 270.2 (M+1)⁺; Retention time: 4.02 minutes. LC method J.

### Step 9: 3-{[(]P)-4-[(2R)-2-Amino-3-(propan-2-yloxy)propoxy]-5-methyl-6-[2-methyl-6-(2-methylpropyl)phenyl]pyrimidin-2-yl]sulfamoyl}benzoic acid

### E30556-1771 Johnny Uy

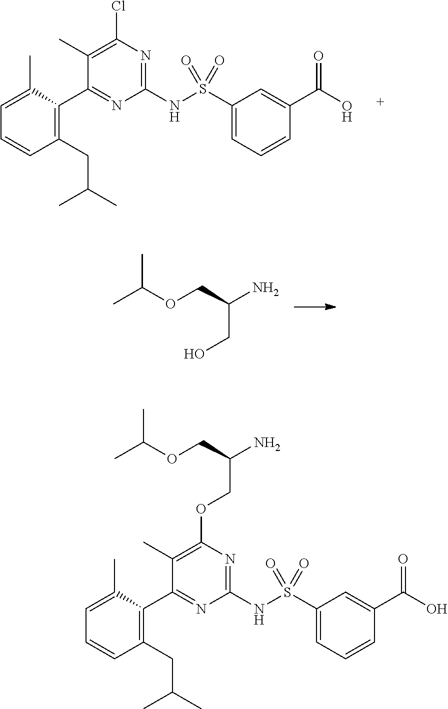

In a round bottom flask, (5P)-3-[[4-Chloro-6-(2-isobutyl-6-methyl-phenyl)-5-methyl-pyrimidin-2-yl]sulfamoyl]benzoic acid (500 mg, 1.055 mmol) and (2S)-2-amino-3-isopropoxy-propan-1-ol (hydrochloride salt) (197 mg, 1.161 mmol) were combined in THE (7.5 mL). In a room temperature water bath, sodium tert-butoxide (Sodium salt) (608 mg, 6.327 mmol) was added in one portion. The reaction mixture was stirred for 1.5 hours at room temperature. The reaction mixture was cooled to 0° C. and quenched with ice cold aqueous 1 M HCl (150 mL). After stirring, the mixture was extracted with EtOAc (4×100 mL). Organic layers were combined and concentrated under reduced pressure. The obtained white solid was taken up in a 4:1 mixture of DCM/iPrOH (100 mL). It was washed with brine (100 mL). The aqueous layer was extracted with DCM (100 mL). All organic layers were combined, dried over sodium sulfate, filtered and concentrated under reduced pressure. EtOAc (80 mL) was added to the obtained white solid. The suspension was stirred as a slurry for 30 minutes. Solids were collected by vacuum filtration, rinsing with EtOAc (2×5 mL). 3-{[(1P)-4-[(2R)-2-amino-3-(propan-2-yloxy)propoxy]-5-methyl-6-[2-methyl-6-(2-methylpropyl)phenyl]pyrimidin-2-yl]sulfamoyl}benzoic acid (hydrochloride salt) (584 mg, 91%) was obtained as a white solid. ESI-MS m/z calc. 570.2512, found 571.5 (M+1)⁺; Retention time: 1.1 minutes. LC method A.

### Step 10: 3-{[(3P, 3P)-5-Methyl-4-[2-methyl-6-(2-methylpropyl)phenyl]-6-[(2R)-2-[({5-methyl-6-[1-(trifluoromethyl)cyclopropyl]-5H-pyrrolo[2,3-b]pyrazin-3-yl}methyl)amino]-3-(propan-2-yloxy)propoxy]pyrimidin-2-yl]sulfamoyl}benzoic acid

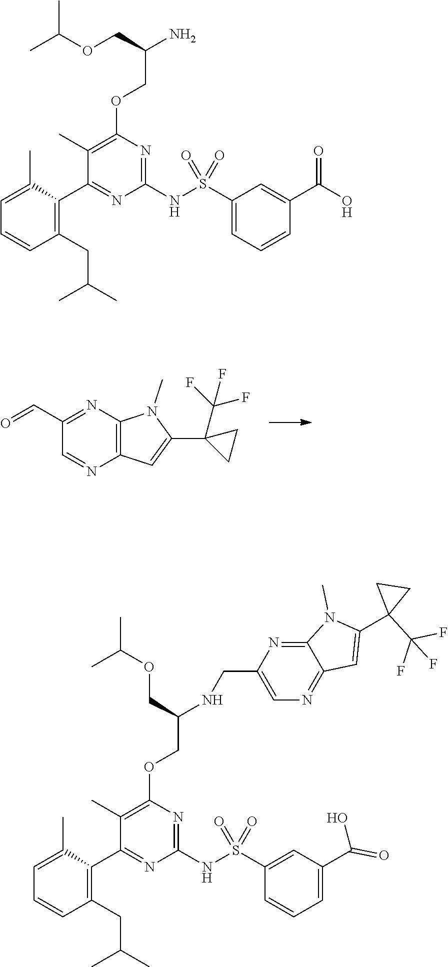

3-{[(1P)-4-[(2R)-2-Amino-3-(propan-2-yloxy)propoxy]-5-methyl-6-[2-methyl-6-(2-methylpropyl)phenyl]pyrimidin-2-yl]sulfamoyl}benzoic acid (hydrochloride salt) (50 mg, 0.08235 mmol) was combined with 5-methyl-6-[1-(trifluoromethyl)cyclopropyl]pyrrolo[2,3-b]pyrazine-3-carbaldehyde (23 mg, 0.08543 mmol) in DCM (0.5 mL). The reaction mixture was stirred for 5 minutes at room temperature. Acetic Acid (10 μL, 0.1758 mmol) was added and the reaction mixture was stirred for an additional 10 minutes at room temperature The reaction mixture was then cooled to 0° C. and DIPEA (40 μL, 0.2296 mmol) was added, followed 5 minutes later by sodium triacetoxyborohydride (120 mg, 0.5662 mmol). The reaction was stirred at 0° C. for one hour. The reaction mixture was then quenched with 0.3 mL 3M HCl and diluted with methanol until the mixture became homogeneous. After filtering the reaction mixture was purified by preparative HPLC (1-99% ACN in water, HCl modifier, 15 minute run) to give 3-{[(3P,3P)-5-methyl-4-[2-methyl-6-(2-methylpropyl)phenyl]-6-[(2R)-2-[({5-methyl-6-[1-(trifluoromethyl)cyclopropyl]-5H-pyrrolo[2,3-b]pyrazin-3-yl}methyl)amino]-3-(propan-2-yloxy)propoxy]pyrimidin-2-yl]sulfamoyl}benzoic acid (hydrochloride salt) (36 mg, 51%) ESI-MS m/z calc. 823.33386, found 824.5 (M+1)⁺; Retention time: 0.67 minutes; LC method B.

### Step 10: (5P,1R)-7-Methyl-6-[2-methyl-6-(2-methylpropyl)phenyl]-12-({5-methyl-6-[1-(trifluoromethyl)cyclopropyl]-5H-pyrrolo[2,3-b]pyrazin-3-yl}methyl)-11-[(propan-2-yloxy)methyl]-9-oxa-2λ⁶-thia-3,5,12,19-tetraazatricyclo[12.3.1.1∧{4,8}]nonadeca-1(18),4(19),5,7,14,16-hexaene-2,2,13-trione (Compound IV-76

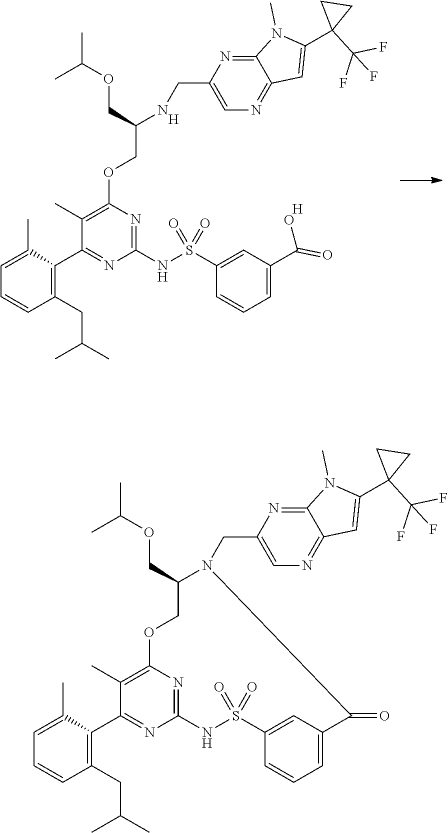

3-{[(3P,3P)-5-Methyl-4-[2-methyl-6-(2-methylpropyl)phenyl]-6-[(2R)-2-[({5-methyl-6-[1-(trifluoromethyl)cyclopropyl]-5H-pyrrolo[2,3-b]pyrazin-3-yl}methyl)amino]-3-(propan-2-yloxy)propoxy]pyrimidin-2-yl]sulfamoyl}benzoic acid (hydrochloride salt) (36 mg, 0.04184 mmol) was combined with CDMT (10 mg, 0.05696 mmol) in a screwcap vial with DMF (3 mL). N-methylmorpholine (40 μL, 0.3638 mmol) was added and the reaction was stirred at room temperature for 3 hours in DMF (3 mL). The reaction mixture was then concentrated by rotary evaporation. After the volatiles were removed the resulting residue was dissolved in 1:1 DMSO/methanol, filtered, and purified by preparative HPLC (1-99% ACN in water, HCl modifier, 15 minute run) to give (5P,11R)-7-methyl-6-[2-methyl-6-(2-methylpropyl)phenyl]-12-({5-methyl-6-[1-(trifluoromethyl)cyclopropyl]-5H-pyrrolo[2,3-b]pyrazin-3-yl}methyl)-11-[(propan-2-yloxy)methyl]-9-oxa-2λ⁶-thia-3,5,12,19-tetraazatricyclo[12.3.1.1∧{4,8}]nonadeca-1(18),4(19),5,7,14,16-hexaene-2,2,13-trione (22.3 mg, 65%). ESI-MS m/z calc. 805.3233, found 806.9 (M+1)⁺; Retention time: 2.25 minutes; LC method A. ¹H NMR (400 MHz, CDCl₃) δ 8.97 (t, J=1.8 Hz, 1H), 8.57 (s, 1H), 8.07 (dt, J=7.9, 1.0 Hz, 1H), 7.87 (dt, J=7.5, 1.3 Hz, 1H), 7.65 (t, J=7.8 Hz, 1H), 7.26-7.22 (m, 1H), 7.12 (d, J=7.7 Hz, 1H), 7.08 (d, J=7.5 Hz, 1H), 6.80 (s, 1H), 5.59-5.54 (m, 1H), 5.51 (d, J=16.1 Hz, 1H), 4.75 (d, J=16.1 Hz, 1H), 4.46-4.34 (m, 2H), 4.02 (s, 3H), 3.71-3.60 (m, 2H), 3.52 (hept, J=6.1 Hz, 1H), 2.22-2.10 (m, 2H), 1.93 (s, 3H), 1.83-1.75 (m, 1H), 1.71 (s, 3H), 1.59 (dt, J=9.3, 4.6 Hz, 2H), 1.24 (q, J=10.0 Hz, 2H), 1.15 (d, J=6.1 Hz, 3H), 1.11 (d, J=6.1 Hz, 3H), 0.80 (dd, J=6.6, 3.4 Hz, 6H).

### Example 125: Preparation of Compound IV-82

### Step 1: 6-tert-Butyl-3-chloro-5-methyl-pyrrolo[2,3-b]pyrazine-7-carbaldehyde

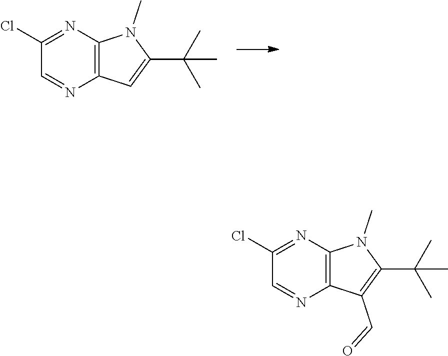 [SMILES: Cn1c(C(C)(C)C)cc2ncc(Cl)nc21 ; Cn1c(C(C)(C)C)c(C=O)c2ncc(Cl)nc21]

To a solution of 6-tert-butyl-3-chloro-5-methyl-pyrrolo[2,3-b]pyrazine (850 mg, 3.7959 mmol) in dimethylformamide (10 mL) was added dropwise phosphorus oxychloride (1.6450 g, 1 mL, 10.728 mmol) at room temperature. The mixture was heated to 70° C. for overnight. The mixture was cooled to room temperature and then phosphorus oxychloride (822.50 mg, 0.5 mL, 5.3642 mmol) was added. The mixture was heated to 70° C. for 5 hours. The mixture was cooled to room temperature and then it was quenched with water (10 mL) dropwise (exothermic). The mixture was purified by reverse phase (acetonitrile in water with 0.1% formic acid 0 to 100%). The fractions containing desired product were combined and concentrated under high vacuum to afford 6-tert-butyl-3-chloro-5-methyl-pyrrolo[2,3-b]pyrazine-7-carbaldehyde (950 mg, 95%) as an light yellow solid; ¹H NMR (400 MHz, CDCl₃) δ 10.69 (s, 1H), 8.55 (s, 1H), 4.10 (s, 3H), 1.71 (s, 9H). ESI-MS m/z calc. 251.0825, found 252.2 (M+1)⁺; Retention time: 1.71 minutes. LC method I.

### Step 2: 6-tert-Butyl-3-chloro-7-[(Z)-2-methoxyvinyl]-5-methyl-pyrrolo[2,3-b]pyrazine and 6-tert-Butyl-3-chloro-7-[(E)-2-methoxyvinyl]-5-methyl-pyrrolo(2,3-pyrazine

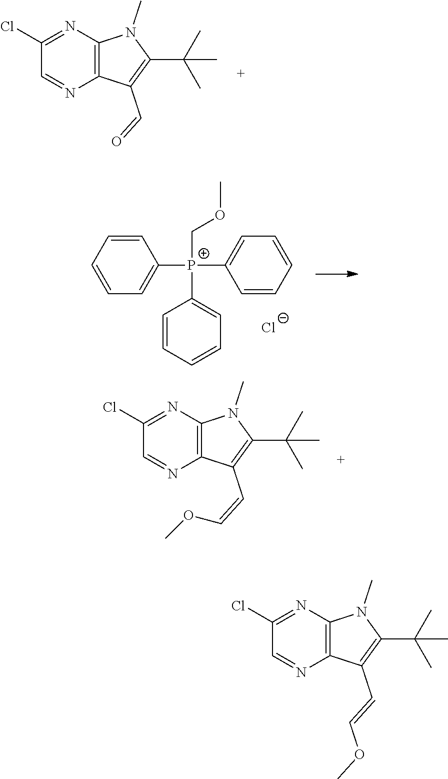 [SMILES: Cn1c(C(C)(C)C)c(C=O)c2ncc(Cl)nc21 ; COC[P+](c1ccccc1)(c1ccccc1)c1ccccc1 ; [Cl-] ; CO/C=C\c1c(C(C)(C)C)n(C)c2nc(Cl)cnc12 ; CO/C=C/c1c(C(C)(C)C)n(C)c2nc(Cl)cnc12]

To a suspension of methoxymethyl(triphenyl)phosphonium chloride (1.5 g, 4.3757 mmol) in THF (15 mL) at 0° C. was added NaHMDS in THF (5.5 mL of 1 M, 5.5000 mmol) dropwise. The mixture was stirred at 0° C. for 45 min. A solution of 6-tert-butyl-3-chloro-5-methyl-pyrrolo[2,3-b]pyrazine-7-carbaldehyde (700 mg, 2.6669 mmol) in THF (8 mL) was added dropwise. The mixture was stirred at 0° C. for 15 min. The mixture was partitioned between ethyl acetate (50 mL) and water (50 mL). The layers were separated, and the aqueous phase was extracted with ethyl acetate (2×50 mL). The combined organic layers were washed with brine (50 mL), dried over sodium sulfate, filtered, and concentrated under reduced pressure. The residue was purified by reverse phase chromatography (5-100% MCN in water with 0.1% formic acid). The fractions containing the first desired Z product were combined and concentrated under high vacuum to afford 6-tert-butyl-3-chloro-7-[(Z)-2-methoxyvinyl]-5-methyl-pyrrolo[2,3-b]pyrazine (230 mg, 30%) as a light brownish yellow oil; ¹H NMR (400 MHz, CDCl₃) δ 8.35 (s, 1H), 6.26 (d, J=6.4 Hz, 1H), 5.47 (d, J=6.6 Hz, 1H), 3.99 (s, 3H), 3.64 (s, 3H), 1.58 (s, 9H). ESI-MS m/z calc. 279.1138, found 280.2 (M+1)⁺; Retention time: 1.91 minutes and The fractions containing the second desired E product were combined and concentrated under high vacuum to afford 6-tert-butyl-3-chloro-7-[(E)-2-methoxyvinyl]-5-methyl-pyrrolo[2,3-b]pyrazine (450 mg, 60%) as a light yellow solid; ¹H NMR (400 MHz, CDCl₃) δ 8.34 (s, 1H), 7.27 (d, J=12.7 Hz, 1H, overlapped with solvent), 6.05 (d, J=12.7 Hz, 1H), 3.99 (s, 3H), 3.74 (s, 3H), 1.61 (s, 9H). ESI-MS m/z calc. 279.1138, found 280.4 (M+1)⁺; Retention time: 2.04 minutes. LC method I.

### Step 3: Methyl 6-tert-butyl-7-[(E)-2-methoxyvinyl]-5-methyl-pyrrolo[2,3-b]pyrazine-3-carboxylate

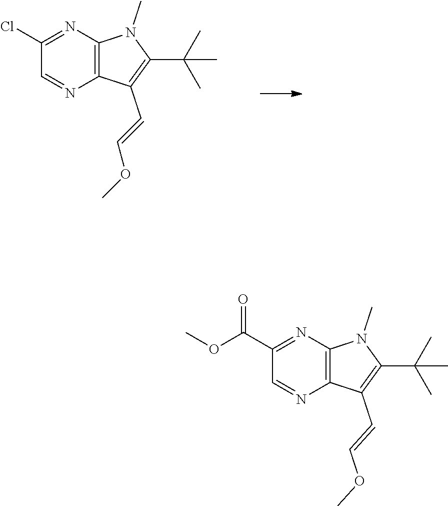 [SMILES: CO/C=C/c1c(C(C)(C)C)n(C)c2nc(Cl)cnc12 ; CO/C=C/c1c(C(C)(C)C)n(C)c2nc(C(=O)OC)cnc12]

To a suspension of 6-tert-butyl-3-chloro-7-[(E)-2-methoxyvinyl]-5-methyl-pyrrolo[2,3-b]pyrazine (445 mg, 1.5890 mmol) in methanol (10 mL) was added triethylamine (471.90 mg, 0.65 mL, 4.6635 mmol). Nitrogen was bubbled into the mixture for 15 minutes and then 1,1′-bis(diphenylphosphino)ferrocene palladium(II) chloride, complex with dichloromethane (65 mg, 0.0796 mmol) was added. Nitrogen bubbling was then continued for 5 minutes. The mixture was then stirred at 100° C. under 50 psi carbon monoxide pressure for 18 hours. The mixture was then allowed to cool down to room temperature, was filtered on a Celite pad and concentrated in vacuo. Water (15 mL) and EtOAc (10 mL) were then added, and the mixture was extracted with EtOAc (3×15 mL). The combined organic layers were washed with brine (25 mL) and dried over anhydrous sodium sulfate, filtered, and concentrated in vacuo. The resulting crude was purified by silica gel chromatography (Gradient: 0-25% EtOAc in heptane) to afford methyl 6-tert-butyl-7-[(E)-2-methoxyvinyl]-5-methyl-pyrrolo[2,3-b]pyrazine-3-carboxylate (400 mg, 83%) as a light yellow solid; ESI-MS m/z calc. 303.1583, found 304.2 (M+1)⁺; Retention time: 1.85 minutes. LC method I.

### Step 4: Methyl 6-tert-butyl-7-(2-methoxyethyl)-5-methyl-pyrrolo[2,3-b]pyrazine-3-carboxylate

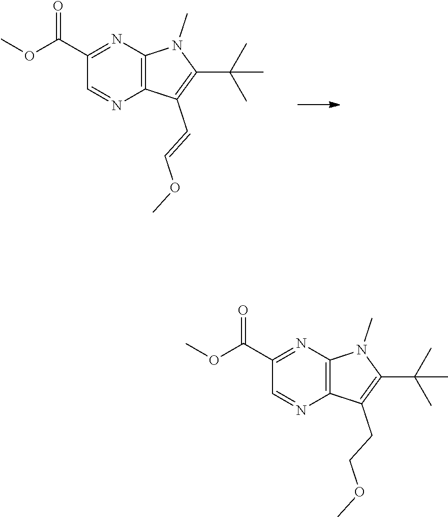 [SMILES: CO/C=C/c1c(C(C)(C)C)n(C)c2nc(C(=O)OC)cnc12 ; COCCc1c(C(C)(C)C)n(C)c2nc(C(=O)OC)cnc12]

A solution of methyl 6-tert-butyl-7-[(E)-2-methoxyvinyl]-5-methyl-pyrrolo[2,3-b]pyrazine-3-carboxylate (400 mg, 1.3173 mmol), methyl 6-tert-butyl-7-[(Z)-2-methoxyvinyl]-5-methyl-pyrrolo[2,3-b]pyrazine-3-carboxylate (300 mg, 0.9128 mmol) and methyl 6-tert-butyl-7-[(E)-2-methoxyvinyl]-5-methyl-pyrrolo[2,3-b]pyrazine-3-carboxylate (51 mg, 0.1680 mmol) in THE (25 mL) was bubbled with nitrogen over 5 minutes and then palladium on carbon (140 mg, 0.1316 mmol) was added. The resulting mixture was bubbled with hydrogen for 10 minutes and then was stirred at room temperature under balloon of hydrogen for 21 hours. The crude was filtered through celite, washed with ethyl acetate (50 mL) and concentrated under reduced pressure to provide crude of methyl 6-tert-butyl-7-(2-methoxyethyl)-5-methyl-pyrrolo[2,3-b]pyrazine-3-carboxylate (635 mg, 82%) as a light yellow solid; ¹H NMR (400 MHz, CDCl₃) δ 9.15 (s, 1H), 4.10 (s, 3H), 4.04 (s, 3H), 3.61 (t, J=7.3 Hz, 2H), 3.42 (t, J=7.8 Hz, 2H), 3.36 (s, 3H), 1.64 (s, 9H). ESI-MS m/z calc. 305.1739, found 306.4 (M+1)⁺; Retention time: 1.93 minutes. LC method I.

### Step 5: 6-tert-Butyl-7-(2-methoxyethyl)-5-methyl-pyrrolo[2,3-b]pyrazine-3-carbaldehyde and[6-tert-butyl-7-(2-methoxyethyl)-5-methyl-pyrrolo[2,3-b]pyrazin-3-yl]methanol

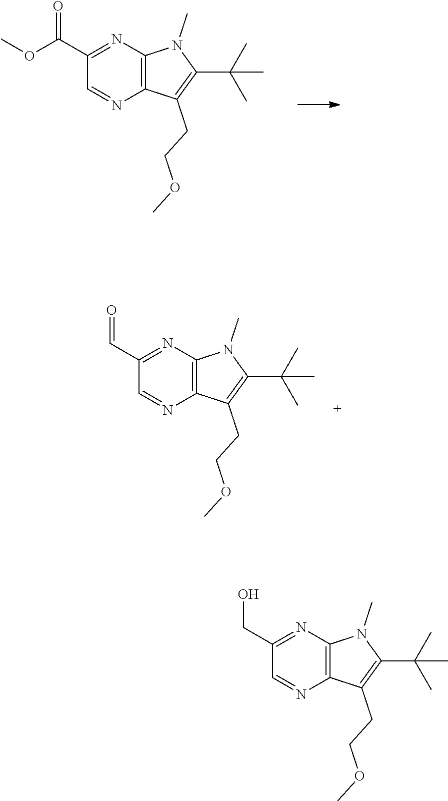 [SMILES: COCCc1c(C(C)(C)C)n(C)c2nc(C(=O)OC)cnc12 ; COCCc1c(C(C)(C)C)n(C)c2nc(C=O)cnc12 ; COCCc1c(C(C)(C)C)n(C)c2nc(CO)cnc12]

To a solution of methyl 6-tert-butyl-7-(2-methoxyethyl)-5-methyl-pyrrolo[2,3-b]pyrazine-3-carboxylate (635 mg, 1.9609 mmol) in dichloromethane (10 mL) at −78° C. was added dropwise DIBAL in dichloromethane (6 mL of 1 M, 6.0000 mmol). The mixture was stirred at −78° C. for 4 hours. the reaction was quenched with methanol/water (1/1) (10 mL). The mixture was partitioned between water (25 mL) and ethyl acetate (25 mL). Phases were separated and then the aqueous phase was extracted with ethyl acetate (2×25 mL). The combined organic layers was washed with brine (50 mL), dried over sodium sulfate, filtered and concentrated under high vacuum to afford a crude mixture (approximate ratio 35:60, total amount 539 mg) of 6-tert-butyl-7-(2-methoxyethyl)-5-methyl-pyrrolo[2,3-b]pyrazine-3-carbaldehyde, ESI-MS m/z calc. 275.1634, found 276.2 (M+1)⁺; Retention time: 1.92 minutes, and [6-tert-butyl-7-(2-methoxyethyl)-5-methyl-pyrrolo[2,3-b]pyrazin-3-yl]methanol as a light brownish oil; ESI-MS m/z calc. 277.179, found 278.2 (M+1)⁺; Retention time: 1.67 minutes. The mixture was used for next step without further purification. LC method I.

### Step 6: 6-tert-Butyl-7-(2-methoxyethyl)-5-methyl-pyrrolo[2,3-b]pyrazine-3-carbaldehyde

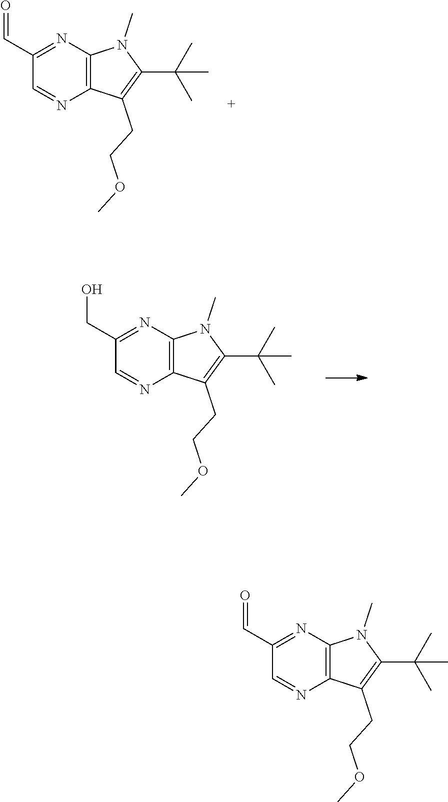 [SMILES: COCCc1c(C(C)(C)C)n(C)c2nc(C=O)cnc12 ; COCCc1c(C(C)(C)C)n(C)c2nc(CO)cnc12 ; COCCc1c(C(C)(C)C)n(C)c2nc(C=O)cnc12]

To a solution of a crude mixture of [6-tert-butyl-7-(2-methoxyethyl)-5-methyl-pyrrolo[2,3-b]pyrazin-3-yl]methanol and 6-tert-butyl-7-(2-methoxyethyl)-5-methyl-pyrrolo[2,3-b]pyrazine-3-carbaldehyde (approximate ratio 60:35, total amount, 450 mg, 1.59 mmol) in dichloromethane (10 mL) at 0° C. was added Dess-Martin periodinane (610 mg, 1.4382 mmol). The mixture was stirred at 0° C. for 15 minutes and then at room temperature for 5 hours. The mixture was partitioned between water (25 mL) and ethyl acetate (25 mL). Phases were separated and the aqueous phase was extracted with ethyl acetate (2×25 mL). The combined organic layers were washed with brine (50 mL), dried over sodium sulfate, filtered, and concentrated under high vacuum. The crude was purified by reverse phase chromatography (Gradient: 0 to 95% acetonitrile in water). The fractions containing the desired product were combined and concentrated under high vacuum to afford 6-tert-butyl-7-(2-methoxyethyl)-5-methyl-pyrrolo[2,3-b]pyrazine-3-carbaldehyde (350 mg, 81%) as a light yellow solid; ¹H NMR (400 MHz, DMSO-d6) δ 10.09 (s, 1H), 8.92 (s, 1H), 4.06 (s, 3H), 3.55-3.45 (m, 2H), 3.34-3.27 (m, 2H), 3.24 (s, 3H), 1.61 (s, 9H). ESI-MS m/z calc. 275.1634, found 276.2 (M+1)⁺; Retention time: 4.25 minutes. LC method J.

### Step 7: 3-[[4-[(2R)-3-(1-Bicyclo[1.1.1]pentanyl)-2-[[6-tert-butyl-7-(2-methoxyethyl)-5-methyl-pyrrolo[2,3-b]pyrazin-3-yl]methylamino]propoxy]-6-(2,6-dimethylphenyl)pyrimidin-2-yl]sulfamoyl]benzoic acid

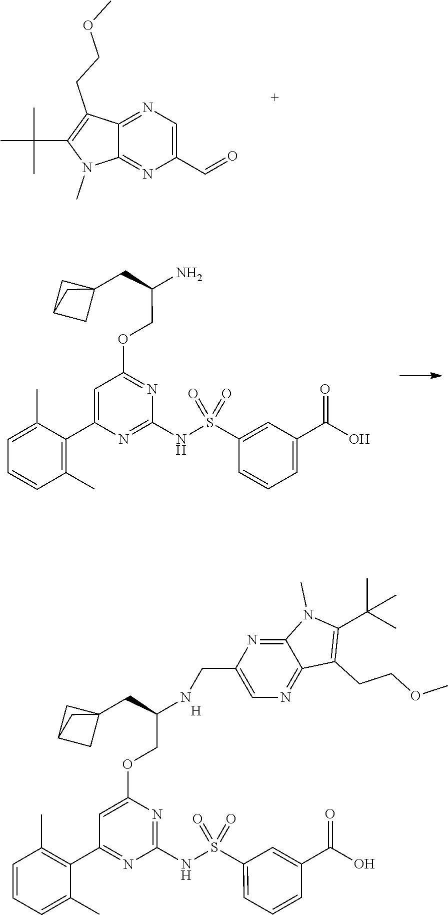

In a 4 mL vial, 6-tert-butyl-7-(2-methoxyethyl)-5-methyl-pyrrolo[2,3-b]pyrazine-3-carbaldehyde (12 mg, 0.04358 mmol) and 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (approximately 29.78 mg, 0.04794 mmol) were combined in DCM (5 mL). Glacial acetic acid (approximately 5.234 mg, 4.956 μL, 0.08716 mmol) was added. The mixture was cooled to 0° C. before the addition of DIEA (approximately 16.89 mg, 22.76 μL, 0.1307 mmol). After stirring for 5 minutes, sodium triacetoxyborohydride (approximately 46.18 mg, 0.2179 mmol) was added. The reaction mixture was stirred at 0° C. for 45 minutes. The reaction was quenched with the addition of methanol and aqueous 1 M HCl. Purification by reverse phase HPLC (10-99% acetonitrile/5 mM aqueous HCl over 15 minutes) provided 3-[[4-[(2R)-3-(1-bicyclo[1.1.1]pentanyl)-2-[[6-tert-butyl-7-(2-methoxyethyl)-5-methyl-pyrrolo[2,3-b]pyrazin-3-yl]methylamino]propoxy]-6-(2,6-dimethylphenyl)pyrimidin-2-yl]sulfamoyl]benzoic acid (hydrochloride salt) (25 mg, 70%). ESI-MS m/z calc. 781.3622, found 782.6 (M+1)⁺; Retention time: 1.47 minutes; LC method A.

### Step 8: (11R)-11-(1-Bicyclo[1.1.1]pentanylmethyl)-12-[[6-tert-butyl-7-(2-methoxyethyl)-5-methyl-pyrrolo[2,3-b]pyrazin-3-yl]methyl]-6-(2,6-dimethylphenyl)-2,2-dioxo-9-oxa-226-thia-3,5,12,19-tetrazatricyclo[12.3.1.14,8]nonadeca-1(18),4(19),5,7,14,16-hexaen-13-one (Compound IV-82

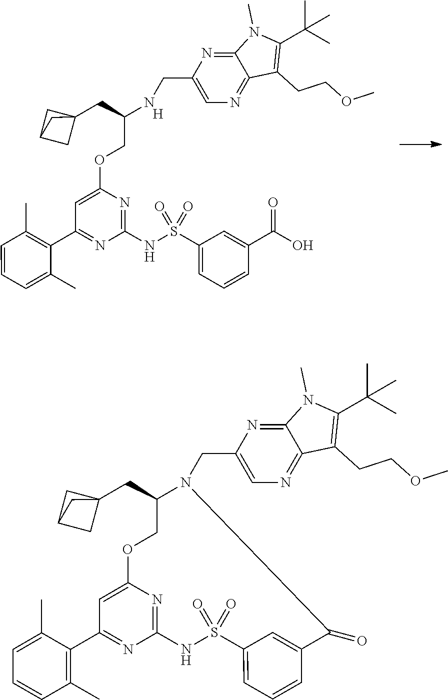

In a 4 mL vial, 3-[[4-[(2R)-3-(1-bicyclo[1.1.1]pentanyl)-2-[[6-tert-butyl-7-(2-methoxyethyl)-5-methyl-pyrrolo[2,3-b]pyrazin-3-yl]methylamino]propoxy]-6-(2,6-dimethylphenyl)pyrimidin-2-yl]sulfamoyl]benzoic acid (hydrochloride salt) (25 mg, 0.03055 mmol) and 2-chloro-4,6-dimethoxy-1,3,5-triazine (approximately 8.045 mg, 0.04582 mmol) were combined in DMF (0.5 mL). The mixture was cooled to 0° C. before the addition of 4-methylmorpholine (approximately 15.46 mg, 16.80 μL, 0.1528 mmol). The reaction mixture was allowed to slowly warm to room temperature and stirred for 2 hours. Purification by reverse phase HPLC (10-99% acetonitrile/5 mM aqueous HCl over 15 minutes) provided (11R)-11-(1-bicyclo[1.1.1]pentanylmethyl)-12-[[6-tert-butyl-7-(2-methoxyethyl)-5-methyl-pyrrolo[2,3-b]pyrazin-3-yl]methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (10.4 mg, 44%). ESI-MS m/z calc. 763.35156, found 764.6 (M+1)⁺; Retention time: 1.92 minutes; LC method A.

### Example 126: Preparation of Compound IV-89

### Step 1: (6-tert-Butyl-3-chloro-5-methyl-pyrrolo[2,3-b]pyrazin-7-yl)methanol

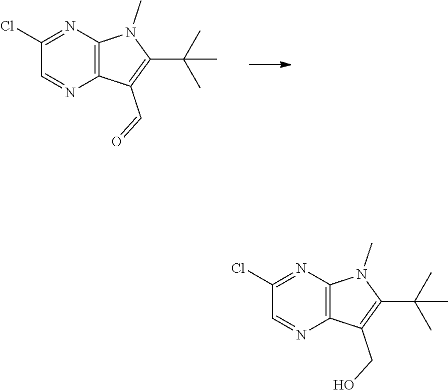 [SMILES: Cn1c(C(C)(C)C)c(C=O)c2ncc(Cl)nc21 ; Cn1c(C(C)(C)C)c(CO)c2ncc(Cl)nc21]

To a solution of 6-tert-butyl-3-chloro-5-methyl-pyrrolo[2,3-b]pyrazine-7-carbaldehyde (792 mg, 2.9891 mmol) in methanol (15 mL) at −5° C. (brine/ice) was added slowly sodium borohydride (121 mg, 3.1983 mmol). The reaction was stirred at −5° C. for 30 minutes. The reaction mixture was quenched with brine (40 mL). The reaction mixture was extracted with EtOAc (3×60 mL). The organic phases were combined then dried over anhydrous sodium sulfate. The solvent was removed under reduced pressure to give crude (6-tert-butyl-3-chloro-5-methyl-pyrrolo[2,3-b]pyrazin-7-yl)methanol (855 mg, 106%) as a beige solid. ¹H NMR (400 MHz, CDCl₃) δ 8.32 (s, 1H), 5.17 (s, 2H), 4.02 (s, 3H), 2.81-2.64 (m, 1H), 1.65 (s, 9H). ESI-MS m/z calc. 253.0982, found 254.2 (M+1)⁺; Retention time: 1.7 minutes, LC method I.

### Step 2: 6-tert-Butyl-3-chloro-7-(methoxymethyl)-5-methyl-pyrrolo[2,3-b]pyrazine

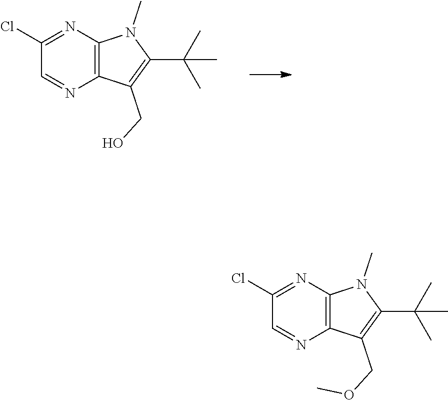 [SMILES: Cn1c(C(C)(C)C)c(CO)c2ncc(Cl)nc21 ; COCc1c(C(C)(C)C)n(C)c2nc(Cl)cnc12]

To a solution of (6-tert-butyl-3-chloro-5-methyl-pyrrolo[2,3-b]pyrazin-7-yl)methanol (855 mg, 3.1743 mmol) in DMF (18 mL) at 0° C. was added NaH (60%, 330 mg, 8.2508 mmol). The solution was stirred for 45 minutes at 0° C. Mel (1.5276 g, 0.67 mL, 10.762 mmol) was added dropwise to the reaction then the temperature was increased slowly to RT for one hour. The reaction was quenched with a saturated aqueous solution of ammonium chloride (10 mL) and diluted with diethyl ether (30 mL). The organic phase was washed with water (2×20 mL) and brine (15 mL). The organic phase was dried over sodium sulfate, filtered and concentrated to give crude 6-tert-butyl-3-chloro-7-(methoxymethyl)-5-methyl-pyrrolo[2,3-b]pyrazine (985 mg, 114%) as a beige solid. ¹H NMR (400 MHz, CDCl₃) δ 8.37 (s, 1H), 4.84 (s, 2H), 4.02 (s, 3H), 3.39 (s, 3H), 1.65 (s, 9H). ESI-MS m/z calc. 267.1138, found 268.2 (M+1)⁺; Retention time: 1.95 minutes, LC method I.

### Step 3: Methyl 6-tert-butyl-7-(methoxymethyl)-5-methyl-pyrrolo[2,3-b]pyrazine-3-carboxylate

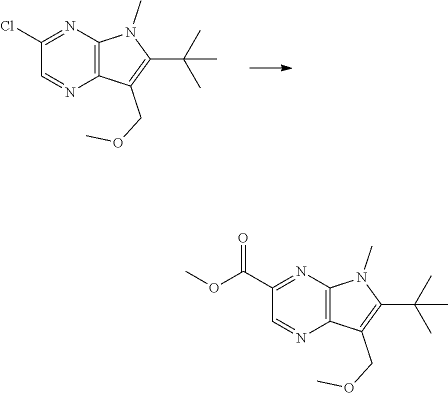 [SMILES: COCc1c(C(C)(C)C)n(C)c2nc(Cl)cnc12 ; COCc1c(C(C)(C)C)n(C)c2nc(C(=O)OC)cnc12]

In a seal tube, a solution of 6-tert-butyl-3-chloro-7-(methoxymethyl)-5-methyl-pyrrolo[2,3-b]pyrazine (1.097 g, 3.7775 mmol) in methanol (20 mL) was mixed with triethylamine (871.20 mg, 1.2 mL, 8.6095 mmol). The system was purged with nitrogen then 1,1′-bis(diphenylphosphino)ferrocene palladium(II) chloride, complex with dichloromethane (168 mg, 0.2057 mmol) was added. The reaction was stirred at 100° C. under carbon monoxide atmosphere (50 psi). The mixture was stirred overnight at 100° C., then cooled down to 25° C. The mixture was purged with nitrogen and concentrated under vacuum. The resulting solid was diluted with ethyl acetate (40 mL) and water (15 mL) The layers were separated. The aqueous phase was extracted with EtOAc (2×25 mL). The organic layers were combined then were washed with water (15 mL) and brine (15 mL), dried over sodium sulfate, filtered, and evaporated under reduced pressure. The residue was purified by chromatography on a silica gel cartridge, eluting from 10% to 60% of ethyl acetate in heptanes to give methyl 6-tert-butyl-7-(methoxymethyl)-5-methyl-pyrrolo[2,3-b]pyrazine-3-carboxylate (1.023 g, 93%) as a beige solid. ¹H NMR (400 MHz, CDCl₃) δ 9.19 (s, 1H), 4.90 (s, 2H), 4.13 (s, 3H), 4.04 (s, 3H), 3.41 (s, 3H), 1.68 (s, 9H). ESI-MS m/z calc. 291.1583, found 292.2 (M+1)⁺; Retention time: 1.75 minutes, LC method I.

### Step 4: [6-tert-Butyl-7-(methoxymethyl)-5-methyl-pyrrolo[2,3-b]pyrazin-3-yl]methanol

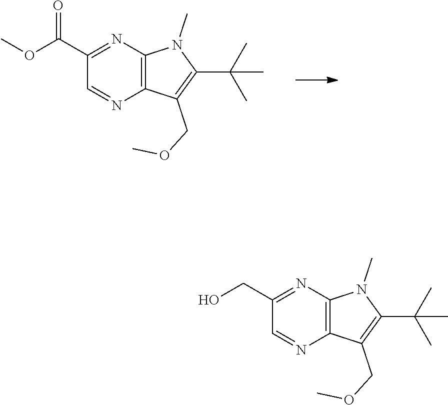 [SMILES: COCc1c(C(C)(C)C)n(C)c2nc(C(=O)OC)cnc12 ; COCc1c(C(C)(C)C)n(C)c2nc(CO)cnc12]

A solution of methyl 6-tert-butyl-7-(methoxymethyl)-5-methyl-pyrrolo[2,3-b]pyrazine-3-carboxylate (1.023 g, 3.5078 mmol) in DCM (40 mL) was cooled at −78° C. then a solution of DIBAL in DCM (11 mL of 1 M, 11.000 mmol) was added. The cooling bath was removed, and the reaction was stirred at rt for 2 h. The reaction was cooled to 0° C. and methanol-water (1:1, 5 mL) was added. The mixture was stirred vigorously for 5 minutes then warmed to room temperature. EtOAc (5 mL) was added to the reaction, then concentrated in vacuo to remove MeOH. The residue was diluted with EtOAc (75 mL), washed with water (20 mL), brine (20 mL), dried over sodium sulfate, filtered and concentrated in vacuo to give crude [6-tert-butyl-7-(methoxymethyl)-5-methyl-pyrrolo[2,3-b]pyrazin-3-yl]methanol (724 mg, 63%) as a beige solid. ¹H NMR (400 MHz, CDCl₃) δ 8.39 (s, 1H), 4.93-4.85 (m, 4H), 4.05 (s, 3H), 3.39 (s, 3H), 1.66 (s, 9H), 1 proton missing (labile proton). ESI-MS m/z calc. 263.1634, found 264.2 (M+1)⁺; Retention time: 1.52 minutes, LC method I.

### Step 5: 6-tert-Butyl-7-(methoxymethyl)-5-methyl-pyrrolo[2,3-b]pyrazine-3-carbaldehyde

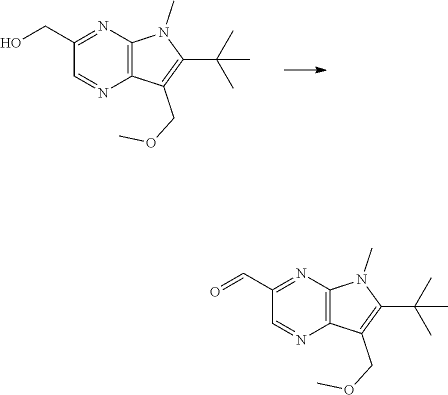 [SMILES: COCc1c(C(C)(C)C)n(C)c2nc(CO)cnc12 ; COCc1c(C(C)(C)C)n(C)c2nc(C=O)cnc12]

To a stirred solution of [6-tert-butyl-7-(methoxymethyl)-5-methyl-pyrrolo[2,3-b]pyrazin-3-yl]methanol (724 mg, 2.2270 mmol) in DCM (100 mL) at 0° C. was added Dess-Martin periodinane (1.42 g, 3.3479 mmol). The reaction was stirred 30 minutes then at RT for 2 hours. The reaction mixture was then quenched by the addition of a 10% aqueous solution of sodium thiosulfate (30 mL), water (12 mL), saturated aqueous sodium bicarbonate (20 mL) and 1 N aqueous sodium hydroxide to adjust pH until 9 (around 15 mL). The biphasic mixture was vigorously stirred at room temperature for 15 minutes. The layers were then separated, and the aqueous layer was extracted with dichloromethane (3×30 mL). The combined organic layers were washed with water (25 mL), dried over sodium sulfate, and filtered. The residue was purified by chromatography on a silica gel cartridge, eluting from 0% to 30% of ethyl acetate in heptanes to give 6-tert-butyl-7-(methoxymethyl)-5-methyl-pyrrolo[2,3-b]pyrazine-3-carbaldehyde (283 mg, 46%) ¹H NMR (400 MHz, CDCl₃) δ 10.17 (s, 1H), 9.07 (s, 1H), 4.90 (s, 2H), 4.14 (s, 3H), 3.41 (s, 3H), 1.69 (s, 9H). as a yellow oil. ESI-MS m/z calc. 261.1477, found 262.2 (M+1)⁺; Retention time: 4.12 minutes. LC method J.

### Step 6: 3-[[4-[(2R)-3-(1-Bicyclo[1.1.1]pentanyl)-2-[[6-tert-butyl-7-(methoxymethyl)-5-methyl-pyrrolo[2,3-b]pyrazin-3-yl]methylamino]propoxy]-6-(2,6-dimethylphenyl)pyrimidin-2-yl]sulfamoyl]benzoic acid

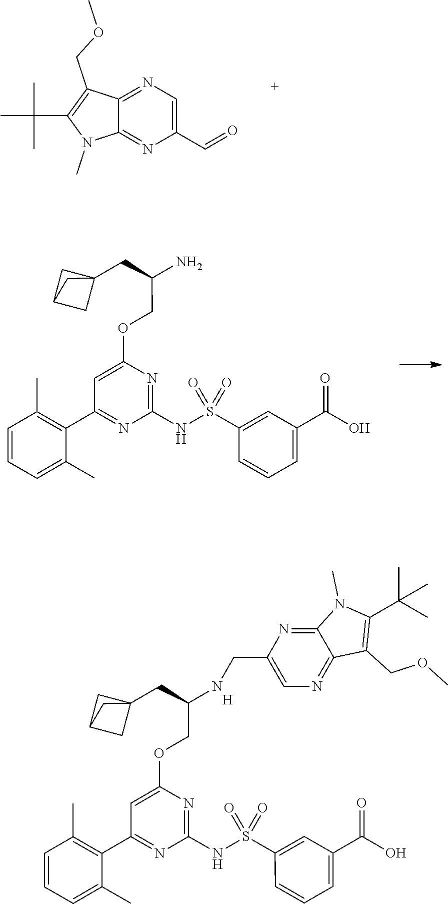

In a 4 mL vial, 6-tert-butyl-7-(methoxymethyl)-5-methyl-pyrrolo[2,3-b]pyrazine-3-carbaldehyde (12 mg, 0.04592 mmol) and 3-[[4-[(2R)-2-amino-3-(1-bicyclo[1.1.1]pentanyl)propoxy]-6-(2,6-dimethylphenyl)pyrimidin-2-yl]sulfamoyl]benzoic acid (hydrochloride salt) (approximately 25.93 mg, 0.04175 mmol) were combined in DCM (0.5 mL). Glacial acetic acid (approximately 5.013 mg, 4.747 μL, 0.08348 mmol) was added. The mixture was cooled to 0° C. before the addition of DIEA (approximately 16.18 mg, 21.81 μL, 0.1252 mmol). After stirring for 10 minutes, sodium triacetoxyborohydride (approximately 44.23 mg, 0.2087 mmol) was added. Stirring was continued at 0° C. for 1 hours. Purification by reverse phase HPLC (10-99% acetonitrile/5 mM aqueous HCl over 15 minutes) provided 3-[[4-[(2R)-3-(1-bicyclo[1.1.1]pentanyl)-2-[[6-tert-butyl-7-(methoxymethyl)-5-methyl-pyrrolo[2,3-b]pyrazin-3-yl]methylamino]propoxy]-6-(2,6-dimethylphenyl)pyrimidin-2-yl]sulfamoyl]benzoic acid (hydrochloride salt) (24 mg, 65%). ESI-MS m/z calc. 767.3465, found 768.6 (M+1)⁺; Retention time: 1.43 minutes; LC method A.

### Step 7: (11R)-11-(1-Bicyclo[1.1.1]pentanylmethyl)-12-[[6-tert-butyl-7-(methoxymethyl)-5-methyl-pyrrolo[2,3-b]pyrazin-3-yl]methyl]-6-(2,6-dimethylphenyl)-2,2-dioxo-9-oxa-226-thia-3,5,12,19-tetrazatricyclo[12.3.1.14,8]nonadeca-1(18),4(19),5,7,14,16-hexaen-13-one (Compound IV-89

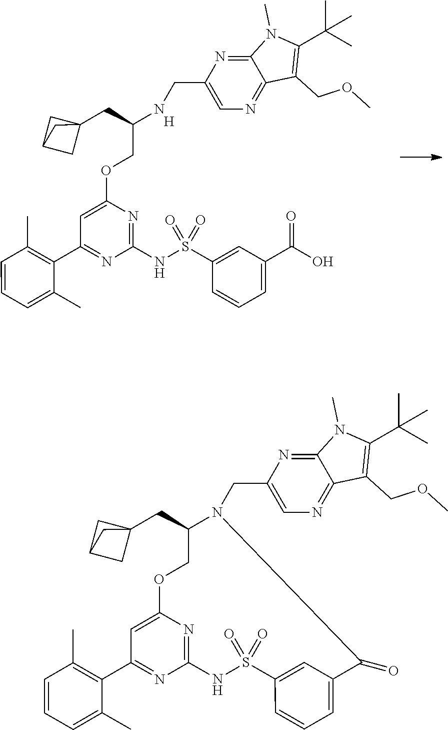

In a 4 mL vial, 3-[[4-[(2R)-3-(1-bicyclo[1.1.1]pentanyl)-2-[[6-tert-butyl-7-(methoxymethyl)-5-methyl-pyrrolo[2,3-b]pyrazin-3-yl]methylamino]propoxy]-6-(2,6-dimethylphenyl)pyrimidin-2-yl]sulfamoyl]benzoic acid (hydrochloride salt) (24 mg, 0.02984 mmol) and 2-chloro-4,6-dimethoxy-1,3,5-triazine (approximately 7.859 mg, 0.04476 mmol) were combined in DMF (0.5 mL). The mixture was cooled to 0° C. before the addition of 4-methylmorpholine (approximately 15.09 mg, 16.40 μL, 0.1492 mmol). The reaction mixture was allowed to slowly warm to room temperature and stirred for 3 hours. Purification by reverse phase HPLC (10-99% acetonitrile/5 mM aqueous HCl over 15 minutes) provided (11R)-11-(1-bicyclo[1.1.1]pentanylmethyl)-12-[[6-tert-butyl-7-(methoxymethyl)-5-methyl-pyrrolo[2,3-b]pyrazin-3-yl]methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.4 mg, 22%). ESI-MS m/z calc. 749.33594, found 750.5 (M+1)⁺; Retention time: 1.91 minutes; LC method A.

### Example 127: Preparation of Compound IV-92

### Step 1: tert-Butyl N-tert-butoxycarbonyl-N-[4-chloro-6-(2,6-diethylphenyl)pyrimidin-2-yl]carbamate

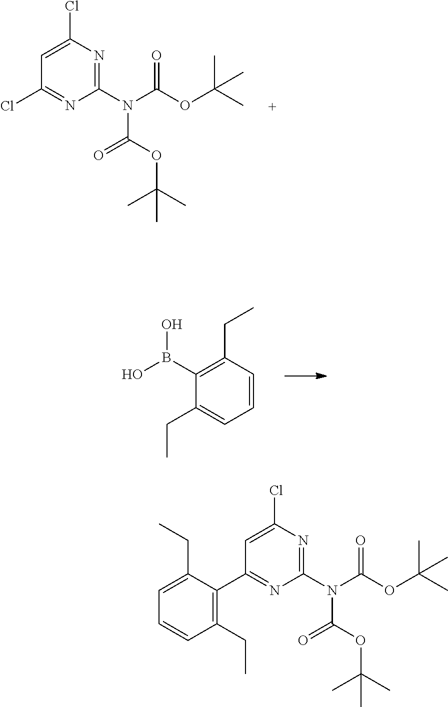 [SMILES: CC(C)(C)OC(=O)N(C(=O)OC(C)(C)C)c1nc(Cl)cc(Cl)n1 ; CCc1cccc(CC)c1B(O)O ; CCc1cccc(CC)c1-c1cc(Cl)nc(N(C(=O)OC(C)(C)C)C(=O)OC(C)(C)C)n1]

In a 4 mL vial, tert-butyl N-tert-butoxycarbonyl-N-(4,6-dichloropyrimidin-2-yl)carbamate (1.65 g, 4.530 mmol) and (2,6-diethylphenyl)boronic acid (806 mg, 4.527 mmol) were combined in DME (12.4 mL). An aqueous solution of cesium carbonate (3.1 mL of 2 M, 6.200 mmol) was added. [1,1-Bis(diphenylphosphino)ferrocene]dichloropalladium, complex with dichloromethane (186 mg, 0.2278 mmol) was added last under nitrogen gas. The reaction mixture was stirred overnight at 85° C. After cooling to room temperature, it was diluted with EtOAc (75 mL) and washed with water (1×75 mL) and brine (1×75 mL). The organic layer was dried over sodium sulfate, filtered, and concentrated under reduced pressure. Purification by flash chromatography on silica gel (0-10% EtOAc/hexanes over 30 minutes) provided tert-butyl N-tert-butoxycarbonyl-N-[4-chloro-6-(2,6-diethylphenyl)pyrimidin-2-yl]carbamate (1.35 g, 65%) as a clear colorless oil. ESI-MS m/z calc. 461.20813, found 462.3 (M+1)⁺; Retention time: 2.09 minutes. LC method A.

### Step 2: 4-Chloro-6-(2,6-diethylphenyl)pyrimidin-2-amine

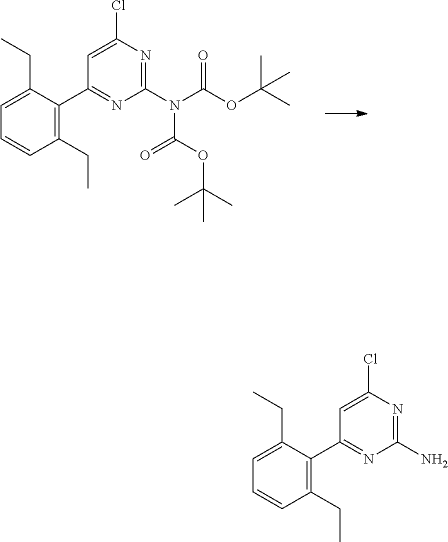 [SMILES: CCc1cccc(CC)c1-c1cc(Cl)nc(N(C(=O)OC(C)(C)C)C(=O)OC(C)(C)C)n1 ; CCc1cccc(CC)c1-c1cc(Cl)nc(N)n1]

A solution of tert-butyl N-tert-butoxycarbonyl-N-[4-chloro-6-(2,6-diethylphenyl)pyrimidin-2-yl]carbamate (1.35 g, 2.922 mmol) in 1,4-dioxane (5.5 mL) was cooled to 0° C. before the dropwise addition of HCl in 1,4-dioxane (4.0 mL of 4 M, 16.00 mmol). The ice bath was removed, and the reaction mixture was stirred overnight at room temperature. The reaction was quenched with the addition of aqueous NaOH (1 M, 75 mL). The mixture was extracted with EtOAc (2×75 mL). Organic layers were combined, dried over sodium sulfate, filtered, and concentrated under reduced pressure. 4-chloro-6-(2,6-diethylphenyl)pyrimidin-2-amine (611 mg, 80%) was obtained as a clear colorless oil that crystallized upon standing. ESI-MS m/z calc. 261.10327, found 262.2 (M+1)⁺; Retention time: 1.42 minutes. LC method A.

### Step 3: 3-[[4-Chloro-6-(2,6-diethylphenyl)pyrimidin-2-yl]sulfamoyl]benzoic acid

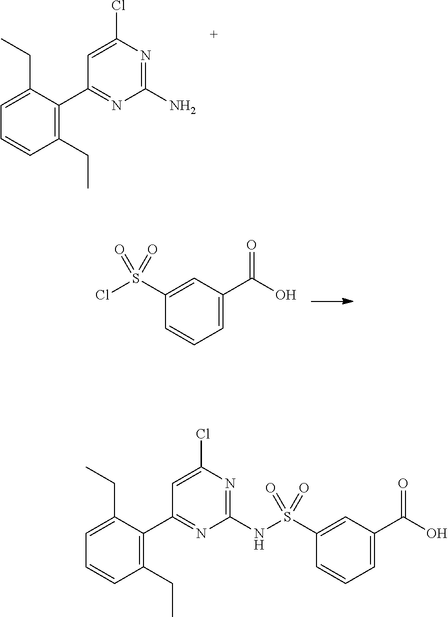 [SMILES: CCc1cccc(CC)c1-c1cc(Cl)nc(N)n1 ; O=C(O)c1cccc(S(=O)(=O)Cl)c1 ; CCc1cccc(CC)c1-c1cc(Cl)nc(NS(=O)(=O)c2cccc(C(=O)O)c2)n1]

In a round bottom flask, 4-chloro-6-(2,6-diethylphenyl)pyrimidin-2-amine (611 mg, 2.334 mmol) was dissolved in THE (6 mL). Solid 2-methylbutan-2-olate (sodium salt) (1.03 g, 9.353 mmol) was added, and the mixture was stirred at 45° C. for 15 minutes. It was then cooled to 0° C. before the slow dropwise addition of a solution of 3-chlorosulfonylbenzoic acid (669 mg, 3.032 mmol) in THE (5 mL). After 30 minutes of stirring, aqueous HCl was added (1 M, 50 mL). It was extracted with EtOAc (2×50 mL). Organic layers were combined, washed with brine, dried over sodium sulfate, filtered, and concentrated under reduced pressure. Purification by flash chromatography on silica gel (0-100% EtOAc/hexanes over 15 minutes) provided 3-[[4-chloro-6-(2,6-diethylphenyl)pyrimidin-2-yl]sulfamoyl]benzoic acid (478 mg, 46%) as a clear colorless oil that started to crystallize upon standing. ESI-MS m/z calc. 445.0863, found 446.2 (M+1)⁺; Retention time: 1.49 minutes. LC method A.

### Step 4: 3-[[4-[(2R)-2-Amino-4-methyl-pentoxy]-6-(2,6-diethylphenyl)pyrimidin-2-yl]sulfamoyl]benzoic acid

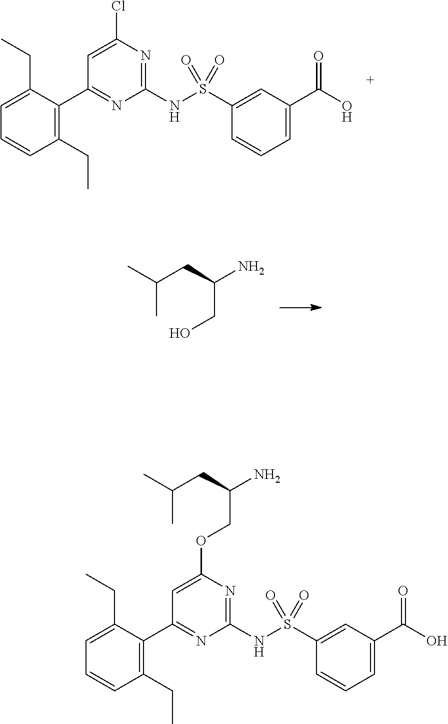

In a round bottom flask, (2R)-2-amino-4-methyl-pentan-1-ol (35 mg, 0.2987 mmol) was dissolved in THE (2.4 mL). HCl in dioxane (370 μL of 4 M, 1.480 mmol) was added. Volatiles were removed under reduced pressure. It was redissolved in THE (2.4 mL), and 3-[[4-chloro-6-(2,6-diethylphenyl)pyrimidin-2-yl]sulfamoyl]benzoic acid (120 mg, 0.2691 mmol) was added. While in a room temperature water bath, solid sodium tert-butoxide (155 mg, 1.613 mmol) was added in one portion. The reaction mixture was stirred at room temperature for 1.5 hours. The reaction mixture was cooled to 0° C. and quenched with ice cold aqueous 1 M HCl (75 mL). After stirring, the mixture was extracted with EtOAc (3×75 mL). Organic layers were combined, washed with brine (1×75 mL), dried over sodium sulfate, filtered, and concentrated under reduced pressure. The crude product was taken up in EtOAc (approximately 3 mL) and triturated with hexane (approximately 10 mL). Solids were collected by vacuum filtration to provide 3-[[4-[(2R)-2-amino-4-methyl-pentoxy]-6-(2,6-diethylphenyl)pyrimidin-2-yl]sulfamoyl]benzoic acid (hydrochloride salt) (99 mg, 65%) as a beige solid. ESI-MS m/z calc. 526.225, found 527.4 (M+1)⁺; Retention time: 1.13 minutes. LC method A.

### Step 5: 3-[[4-[(2R)-2-[[6-tert-Butyl-7-(2-methoxyethyl)-5-methyl-pyrrolo[2,3-b]pyrazin-3-yl]methylamino]-4-methyl-pentoxy]-6-(2,6-diethylphenyl)pyrimidin-2-yl]sulfamoyl]benzoic acid

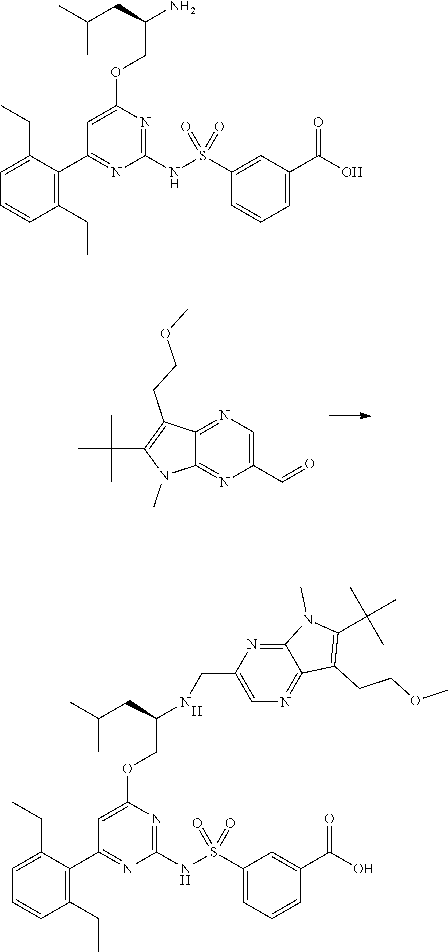

In a 4 mL vial, 3-[[4-[(2R)-2-amino-4-methyl-pentoxy]-6-(2,6-diethylphenyl)pyrimidin-2-yl]sulfamoyl]benzoic acid (hydrochloride salt) (20 mg, 0.03552 mmol) and 6-tert-butyl-7-(2-methoxyethyl)-5-methyl-pyrrolo[2,3-b]pyrazine-3-carbaldehyde (approximately 10.76 mg, 0.03907 mmol) were combined in DCM. Glacial acetic acid (approximately 4.266 mg, 4.040 μL, 0.07104 mmol) was added. The mixture was cooled to 0° C. before the addition of DIEA (approximately 13.78 mg, 18.57 μL, 0.1066 mmol). After stirring for 10 minutes, sodium triacetoxyborohydride (approximately 37.64 mg, 0.1776 mmol) was added. Stirring was continued at 0° C. for 1 hour. The reaction was quenched with the addition of methanol and aqueous 1 M HCl. Purification by reverse phase HPLC (10-99% acetonitrile/5 mM aqueous HCl over 15 minutes) provided 3-[[4-[(2R)-2-[[6-tert-butyl-7-(2-methoxyethyl)-5-methyl-pyrrolo[2,3-b]pyrazin-3-yl]methylamino]-4-methyl-pentoxy]-6-(2,6-diethylphenyl)pyrimidin-2-yl]sulfamoyl]benzoic acid (hydrochloride salt) (14 mg, 48%). ESI-MS m/z calc. 785.39343, found 786.6 (M+1)⁺; Retention time: 1.52 minutes; LC method A.

### Step 6: (11R)-12-[[6-tert-Butyl-7-(2-methoxyethyl)-5-methyl-pyrrolo[2,3-b]pyrazin-3-yl]methyl]-6-(2,6-diethylphenyl)-11-isobutyl-2,2-dioxo-9-oxa-2λ⁶-thia-3,5,12,19-tetraza-tricyclo[12.3.1.14,8]nonadeca-1(18),4(19),5,7,14,16-hexaen-13-one (Compound IV-92

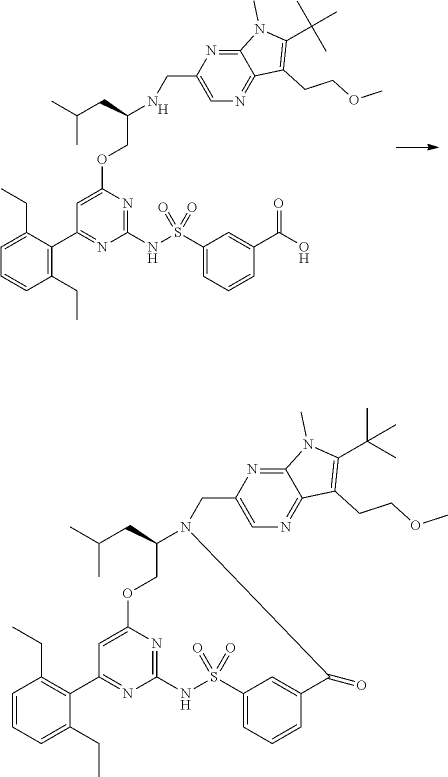

In a 4 mL vial, 3-[[4-[(2R)-2-[[6-tert-butyl-7-(2-methoxyethyl)-5-methyl-pyrrolo[2,3-b]pyrazin-3-yl]methylamino]-4-methyl-pentoxy]-6-(2,6-diethylphenyl)pyrimidin-2-yl]sulfamoyl]benzoic acid (hydrochloride salt) (14 mg, 0.01702 mmol) and CDMT (approximately 4.482 mg, 0.02553 mmol) were combined in DMF (0.5 mL). The mixture was cooled to 0° C. before the addition of 4-methylmorpholine (approximately 8.608 mg, 9.357 μL, 0.08510 mmol). The cooling bath was removed, and the reaction mixture was stirred at room temperature for 2 hours. Purification by reverse phase HPLC (10-99% acetonitrile/5 mM aqueous HCl over 15 minutes) provided (11R)-12-[[6-tert-butyl-7-(2-methoxyethyl)-5-methyl-pyrrolo[2,3-b]pyrazin-3-yl]methyl]-6-(2,6-diethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (2.9 mg, 22%). ESI-MS m/z calc. 767.3829, found 768.7 (M+1)⁺; Retention time: 2.02 minutes; LC method A.

### Example 128: Preparation of Compound IV-93

### Step 1: Methyl 7-fluoro-5-methyl-6-(1-methylcyclopropyl)pyrrolo[2,3-b]pyrazine-3-carboxylate

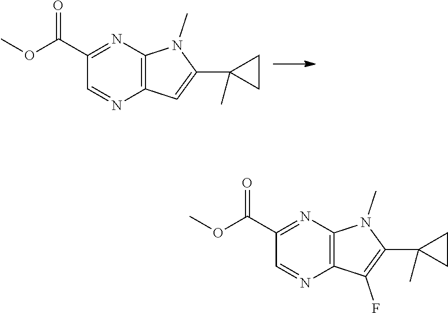 [SMILES: COC(=O)c1cnc2cc(C3(C)CC3)n(C)c2n1 ; COC(=O)c1cnc2c(F)c(C3(C)CC3)n(C)c2n1]

To a solution of methyl 5-methyl-6-(1-methylcyclopropyl)pyrrolo[2,3-b]pyrazine-3-carboxylate (1.84 g, 7.4042 mmol) in acetonitrile (37 mL) were added selectfluor (3.15 g, 8.8918 mmol) and pyridine (4.1244 g, 4.2 mL, 52.142 mmol) at room temperature. The reaction was stirred for 16 h at that temperature. The crude mixture was concentrated in vacuo and was then purified by reverse phase chromatography on a C₁₈ cartridge, eluting with a gradient of 55 to 65% of MeCN in water to afford after evaporation methyl 7-fluoro-5-methyl-6-(1-methylcyclopropyl)pyrrolo[2,3-b]pyrazine-3-carboxylate (612.4 mg, 31%) as a yellow solid. ¹H NMR (400 MHz, CDCl₃) δ 9.18 (s, 1H), 4.05 (s, 3H), 3.99 (s, 3H), 1.49 (s, 3H), 1.14-1.09 (m, 2H), 1.03-0.97 (m, 2H). ESI-MS m/z calc. 263.107, found 264.2 (M+1)⁺; Retention time: 1.73 minutes, LC method I.

### Step 2: 7-Fluoro-5-methyl-6-(1-methylcyclopropyl)pyrrolo[2,3-b]pyrazin-3-yl]methanol and 7-fluoro-5-methyl-6-(1-methylcyclopropyl)pyrrolo[2,3-b]pyrazine-3-carbaldehyde

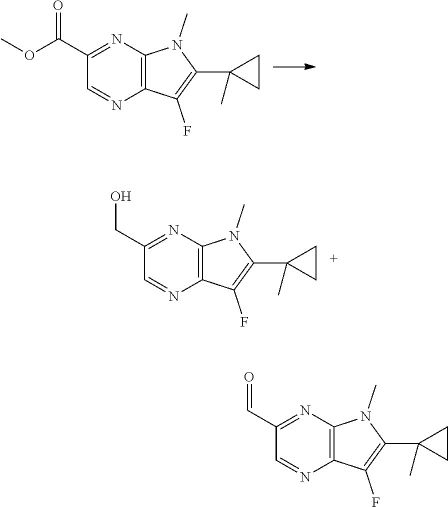 [SMILES: COC(=O)c1cnc2c(F)c(C3(C)CC3)n(C)c2n1 ; Cn1c(C2(C)CC2)c(F)c2ncc(CO)nc21 ; Cn1c(C2(C)CC2)c(F)c2ncc(C=O)nc21]

To a solution of methyl 7-fluoro-5-methyl-6-(1-methylcyclopropyl)pyrrolo[2,3-b]pyrazine-3-carboxylate (511.1 mg, 1.9394 mmol) in dichloromethane (10 mL) at −78° C. was added diisobutylaluminum hydride (in hexanes) (4.3 mL of 1 M, 4.3000 mmol) dropwise. The reaction was stirred at that temperature for 1 hour then the reaction was quenched by a slow addition of EtOAc (20 mL). To the reaction mixture was added a 10% aqueous solution of Rochelle's salt (100 mL) and EtOAc (100 mL). The mixture was vigorously stirred for 15 minutes, and the volatiles were removed under reduced pressure. EtOAc (100 mL) was again added, and the aqueous phase was extracted with EtOAc (3×100 mL). The combined organic layers were washed with brine (100 mL), dried over anhydrous sodium sulfate and filtered to afford 426.7 mg of a mixture (approximate ratio 70:30) of [7-fluoro-5-methyl-6-(1-methylcyclopropyl)pyrrolo[2,3-b]pyrazin-3-yl]methanol, ESI-MS m/z calc. 235.1121, found 236.4 (M+1)⁺; Retention time: 1.56 minutes and 7-fluoro-5-methyl-6-(1-methylcyclopropyl)pyrrolo[2,3-b]pyrazine-3-carbaldehyde, ESI-MS m/z calc. 233.0964, found 234.2 (M+1)⁺; Retention time: 1.74 minutes as a yellow solid. LC method I.

### Step 3: 7-Fluoro-5-methyl-6-(1-methylcyclopropyl)pyrrolo[2,3-b]pyrazine-3-carbaldehyde

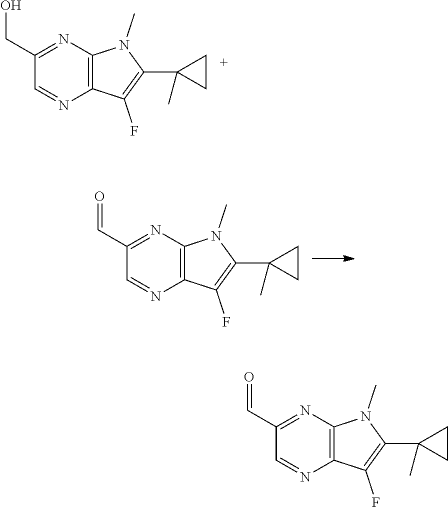 [SMILES: Cn1c(C2(C)CC2)c(F)c2ncc(CO)nc21 ; Cn1c(C2(C)CC2)c(F)c2ncc(C=O)nc21 ; Cn1c(C2(C)CC2)c(F)c2ncc(C=O)nc21]

To a 0° C. solution of the same mixture (426.7 mg) of [7-fluoro-5-methyl-6-(1-methylcyclopropyl)pyrrolo[2,3-b]pyrazin-3-yl]methanol and [7-fluoro-5-methyl-6-(1-methylcyclopropyl)pyrrolo[2,3-b]pyrazin-3-yl]methanol in dichloromethane (20 mL) was added Dess-Martin periodinane (680 mg, 1.6032 mmol) and the reaction was stirred for 1 hour at room temperature. A mixture of aqueous saturated solution of Na₂S₂O₃ (50 mL), saturated sodium bicarbonate (50 mL), water (20 mL) and 1 N NaOH (20 mL, to reach pH=9) was added and the reaction mixture was stirred for 40 minutes. The phases were separated and the aqueous one was extracted with DCM (3×50 mL). The combined organic layers were dried over sodium sulfate and filtered. The crude mixture was concentrated in vacuo and was then purified by flash-chromatography on a silica gel cartridge, eluting with a gradient of 0 to 100% of EtOAc in heptanes to afford after evaporation 7-fluoro-5-methyl-6-(1-methylcyclopropyl)pyrrolo[2,3-b]pyrazine-3-carbaldehyde (380.8 mg, 90%) as a yellow solid. ¹H NMR (400 MHz, CDCl₃) δ 10.16 (s, 1H), 9.06 (s, 1H), 4.01 (s, 3H), 1.51 (s, 3H), 1.16-1.11 (m, 2H), 1.05-1.00 (m, 2H). ESI-MS m/z calc. 233.0964, found 234.2 (M+1)⁺; Retention time: 1.75 minutes, LC method I.

### Step 4: 3-[[4-(2,6-Dimethylphenyl)-6-[(2R)-2-[[7-fluoro-5-methyl-6-(1-methylcyclopropyl) pyrrolo[2,3-b]pyrazin-3-yl]methylamino]-3-(1-methylcyclopropyl)propoxy]pyrimidin-2-yl]sulfamoyl]benzoic acid

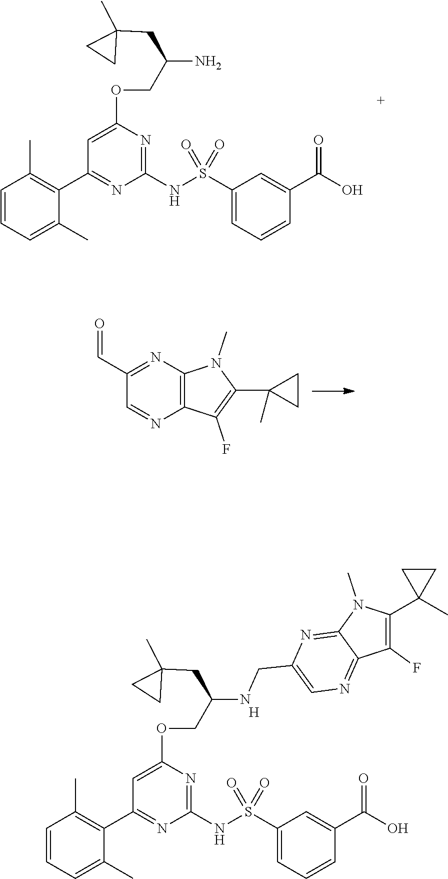

A 4 mL vial was charged under nitrogen with 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (79.2 mg, 0.1448 mmol), 7-fluoro-5-methyl-6-(1-methylcyclopropyl)pyrrolo[2,3-b]pyrazine-3-carbaldehyde (38.8 mg, 0.1663 mmol), anhydrous DCM (0.9 mL), and acetic acid (9.9 μL, 0.1741 mmol). The mixture was cooled down in an ice bath. DIEA (37.9 μL, 0.2176 mmol) was added, followed by sodium triacetoxyborohydride (156.2 mg, 0.7370 mmol), and the reaction was vigorously stirred at 0° C. for 1 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-(2,6-dimethylphenyl)-6-[(2R)-2-[[7-fluoro-5-methyl-6-(1-methylcyclopropyl)pyrrolo[2,3-b]pyrazin-3-yl]methylamino]-3-(1-methylcyclopropyl)propoxy]pyrimidin-2-yl]sulfamoyl]benzoic acid (80.8 mg, 77%) as a yellow solid. ESI-MS m/z calc. 727.2952, found 728.4 (M+1)⁺; Retention time: 1.55 minutes. LC method A.

### Step 5: (11R)-6-(2,6-Dimethylphenyl)-12-[[7-fluoro-5-methyl-6-(1-methylcyclopropyl) pyrrolo[2,3-b]pyrazin-3-yl]methyl]-11-[(1-methylcyclopropyl)methyl]-2,2-dioxo-9-oxa-226-thia-3,5,12,19-tetrazatricyclo[12.3.1.14,8]nonadeca-1(18),4(19),5,7,14,16-hexaen-13-one (Compound IV-93

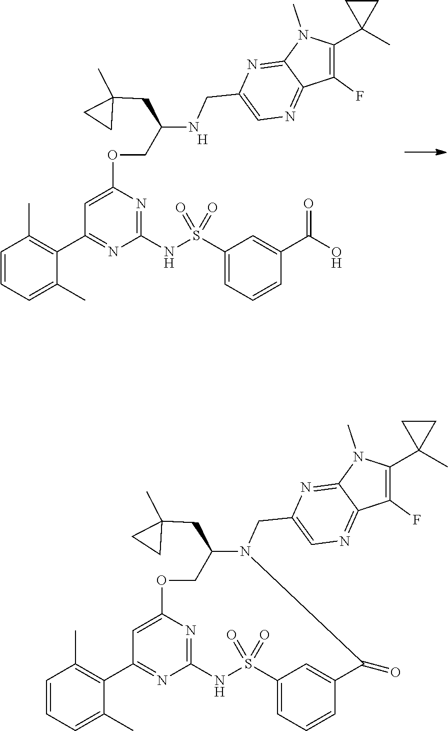

3-[[4-(2,6-Dimethylphenyl)-6-[(2R)-2-[[7-fluoro-5-methyl-6-(1-methylcyclopropyl)pyrrolo[2,3-b]pyrazin-3-yl]methylamino]-3-(1-methylcyclopropyl) propoxy]pyrimidin-2-yl]sulfamoyl]benzoic acid (80.8 mg, 0.1110 mmol) was combined with CDMT (31.0 mg, 0.1766 mmol) in DMF (11 mL) and cooled to 0° C. N-methylmorpholine (36.7 μL, 0.3338 mmol) was added by syringe and the reaction was stirred at 0° C. for 30 minutes. The ice bath was then removed and stirring was continued for an additional 16 hours at room temperature. The reaction mixture was then partitioned between 25 mL 1 M HCl and 25 mL ethyl acetate. The layers were separated, and the aqueous layer was extracted with an additional 25 mL ethyl acetate. The combined organic layers were washed 2×25 mL with brine, dried over sodium sulfate, filtered, and concentrated. The crude product was purified by reverse phase HPLC (1-99% acetonitrile/5 mM aqueous HCl over 25 min) to give (11R)-6-(2,6-dimethylphenyl)-12-[[7-fluoro-5-methyl-6-(1-methylcyclopropyl)pyrrolo[2,3-b]pyrazin-3-yl]methyl]-11-[(1-methylcyclopropyl)methyl]-2,2-dioxo-9-oxa-2λ⁶-thia-3,5,12,19-tetraza-tricyclo[12.3.1.14,8]nonadeca-1(18),4(19),5,7,14,16-hexaen-13-one (15.6 mg, 20%) as a yellow solid. ¹H NMR (400 MHz, CDCl₃) δ 9.00 (t, J=1.8 Hz, 1H), 8.51 (s, 1H), 7.98 (dt, J=8.1, 1.3 Hz, 1H), 7.87 (dt, J=7.7, 1.3 Hz, 1H), 7.62 (t, J=7.8 Hz, 1H), 7.22 (t, J=7.6 Hz, 1H), 7.07 (d, J=7.6 Hz, 2H), 6.24 (s, 1H), 5.58 (dd, J=11.2, 4.3 Hz, 1H), 5.41 (d, J=16.0 Hz, 1H), 4.45-4.35 (m, 1H), 4.24 (d, J=15.9 Hz, 1H), 4.02 (t, J=11.5 Hz, 1H), 3.97 (s, 3H), 2.02 (s, 6H), 1.87-1.83 (m, 1H), 1.50-1.39 (m, 4H), 1.04 (t, J=8.5 Hz, 2H), 0.97-0.86 (m, 2H), 0.54 (s, 3H), 0.44-0.34 (m, 1H), 0.33-0.24 (m, 1H), 0.20-0.11 (m, 1H), 0.07-0.02 (m, 1H). ESI-MS m/z calc. 709.28467, found 710.3 (M+1)⁺; Retention time: 2.12 minutes. LC method A.

### Example 129: Preparation of Compound IV-96

### Step 1: (NE,R)-N-(2-Furylmethylene)-2-methyl-propane-2-sulfinamide

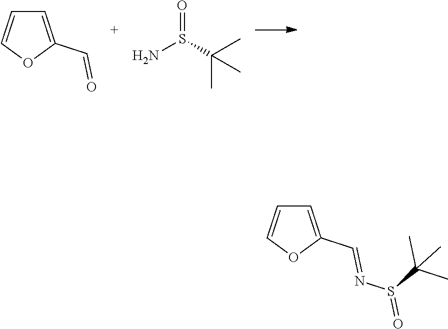

To a solution of furan-2-carbaldehyde (8.1200 g, 7 mL, 84.509 mmol) in toluene (250 mL) was added (R)-2-methylpropane-2-sulfinamide (5 g, 41.254 mmol) and potassium bisulfate (11.5 g, 84.454 mmol). After stirring at 45° C. for 20 hours, the mixture was filtered, and the filtrate was concentrated to dryness. The crude was purified by chromatography on silica gel (Column: 120 g. Gradient: 0-25% ethyl acetate in heptanes) to afford (NE,R)-N-(2-furylmethylene)-2-methyl-propane-2-sulfinamide (5.3 g, 64%) as an brownish oil; ¹H NMR (400 MHz, CDCl₃) δ 8.39 (s, 1H), 7.66-7.60 (m, 1H), 7.00 (d, J=3.4 Hz, 1H), 6.56 (dd, J 3.5, 1.8 Hz, 1H), 1.24 (s, 9H). ESI-MS m/z calc. 199.0667, found 200.2 (M+1)⁺; Retention time: 1.61 minutes. LC method I.

### Step 2: (R)-N-[(1S)-1-(2-Furyl)-2,2-bis(4,4,5,5-tetramethyl-1,3,2-dioxaborolan-2-yl)ethyl]-2-methyl-propane-2-sulfinamide

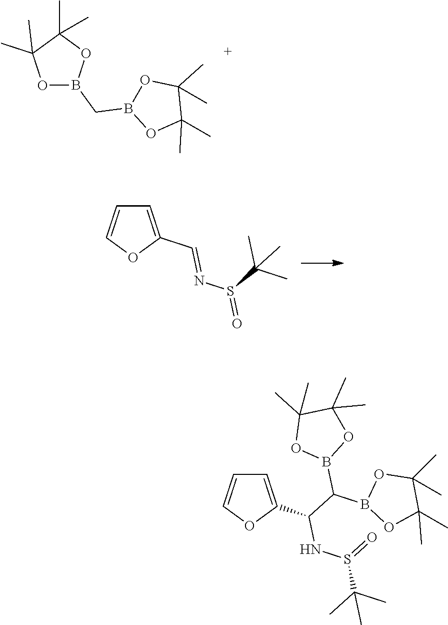

To a solution of TMP (4.3200 g, 4 mL, 30.583 mmol) in THE (25 mL) under nitrogen at 0° C. was added dropwise n-BuLi in hexanes (12 mL of 2.5 M, 30.000 mmol). The reaction mixture was stirred at 0° C. for 30 minutes. A solution of 4,4,5,5-tetramethyl-2-[(4,4,5,5-tetramethyl-1,3,2-dioxaborolan-2-yl)methyl]-1,3,2-dioxaborolane (8.3 g, 30.974 mmol) in THE (25 mL) was added via syringe. The reaction mixture was allowed to stir at 0° C. for 10 minutes. The reaction mixture was then cooled to −78° C., and a solution of (NE,R)-N-(2-furylmethylene)-2-methyl-propane-2-sulfinamide (5.2 g, 25.886 mmol) in THF (15 mL) was added dropwise. The reaction mixture was allowed to stir at −78° C. for another 4 hours. The reaction mixture was quenched by the addition of saturated aqueous ammonium chloride solution (75 mL) at −78° C. The cooling bath was removed, and the aqueous mixture was extracted with DCM (3×50 mL). The combined organic layers were dried over anhydrous sodium sulfate, filtered, and concentrated under reduced pressure. The crude was purified by column chromatography (ethyl acetate in heptanes 0 to 40%). The fractions containing the desired product were combined and concentrated to afford (R)-N-[(1S)-1-(2-furyl)-2,2-bis(4,4,5,5-tetramethyl-1,3,2-dioxaborolan-2-yl)ethyl]-2-methyl-propane-2-sulfinamide (12.1 g, 77%) as a light yellow solid; ESI-MS m/z calc. 467.2684, found 468.2 (M+1)⁺; Retention time: 1.97 minutes. LC method I.

### Step 3: (R)-N-[(1S)-1-(2-furyl)-2-(4,4,5,5-tetramethyl-1,3,2-dioxaborolan-2-yl)ethyl]-2-methyl-propane-2-sulfinamide

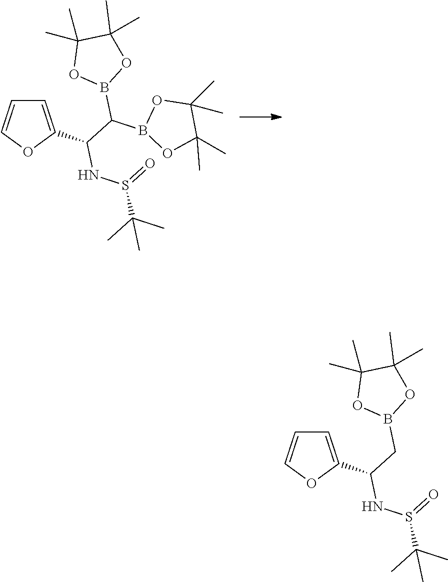 [SMILES: CC(C)(C)[S@@](=O)N[C@H](c1ccco1)C(B1OC(C)(C)C(C)(C)O1)B1OC(C)(C)C(C)(C)O1 ; CC(C)(C)[S@@](=O)N[C@@H](CB1OC(C)(C)C(C)(C)O1)c1ccco1]

To a solution of (R)-N-[(1S)-1-(2-furyl)-2,2-bis(4,4,5,5-tetramethyl-1,3,2-dioxaborolan-2-yl)ethyl]-2-methyl-propane-2-sulfinamide (12.1 g, 19.967 mmol) in acetonitrile (121 mL) was added water (400.00 mg, 0.4 mL, 22.203 mmol) followed by CsF (3.4 g, 22.383 mmol). The mixture was heated to 50° C. for 2 hours. diethyl ether (75 mL) was added. The mixture was filtered through a short pad of Celite, washed with diethyl ether (50 mL) and concentrated under reduced pressure. The crude was purified by column chromatography (ethyl acetate in heptanes 0 to 40%). The fractions containing the desired product were combined and concentrated to provide (R)-N-[(1S)-1-(2-furyl)-2-(4,4,5,5-tetramethyl-1,3,2-dioxaborolan-2-yl)ethyl]-2-methyl-propane-2-sulfinamide (3.6 g, 47%) as a colorless oil, that solidified upon standing; ¹H NMR (400 MHz, CDCl₃) δ 7.35-7.30 (m, 1H), 6.30-6.26 (m, 1H), 6.19-6.15 (m, 1H), 4.68 (q, J=6.4 Hz, 1H), 4.27 (d, J=6.6 Hz, 1H), 1.55 (d, J=6.8 Hz, 2H), 1.23-1.17 (m, 21H). ESI-MS m/z calc. 341.1832, found 342.2 (M+1)⁺; Retention time: 1.8 minutes and a second batch of (R)-N-[(1S)-1-(2-furyl)-2-(4,4,5,5-tetramethyl-1,3,2-dioxaborolan-2-yl)ethyl]-2-methyl-propane-2-sulfinamide (1.1 g, 13%) as an brownish oil, that solidified upon standing; ESI-MS m/z calc. 341.1832, found 342.4 (M+1)⁺; Retention time: 1.8 minutes. LC method I.

### Step 4: (R)-N-[(1S)-1-(2-Furyl)-2-hydroxy-ethyl]-2-methyl-propane-2-sulfinamide

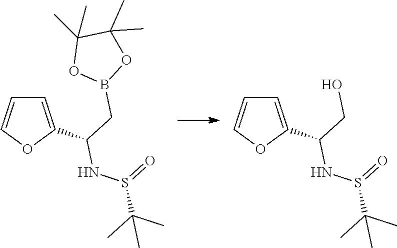 [SMILES: CC(C)(C)[S@@](=O)N[C@@H](CB1OC(C)(C)C(C)(C)O1)c1ccco1 ; CC(C)(C)[S@@](=O)N[C@@H](CO)c1ccco1]

To a solution of (R)-N-[(1S)-1-(2-furyl)-2-(4,4,5,5-tetramethyl-1,3,2-dioxaborolan-2-yl)ethyl]-2-methyl-propane-2-sulfinamide (3.55 g, 9.1955 mmol) in THF (50 mL) and water (50 mL) was added sodium perborate tetrahydrate (7 g, 45.496 mmol). The mixture was stirred at room temperature for 30 minutes. The mixture was partitioned between dichloromethane (50 mL) and water (50 mL). The aqueous phase was extracted with dichloromethane (3×30 mL). The combined organic layers were dried over sodium sulfate, filtered, and concentrated in vacuo. The crude was purified by column chromatography (ethyl acetate in dichloromethane 0 to 50%). The fractions containing the desired product were combined and concentrated under high vacuum to provide (R)-N-[(1S)-1-(2-furyl)-2-hydroxy-ethyl]-2-methyl-propane-2-sulfinamide (1.85 g, 84%) as a colorless oil; ESI-MS m/z calc. 231.0929, found 232.2 (M+1)⁺; Retention time: 1.47 minutes. LC method I.

### Step 5: (2S)-2-Amino-2-(2-furyl)ethanol

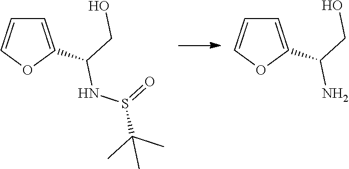

To a solution of (R)-N-[(1S)-1-(2-furyl)-2-hydroxy-ethyl]-2-methyl-propane-2-sulfinamide (1.85 g, 7.7659 mmol) in methanol (40 mL) was added HCl in dioxane (10 mL of 4 M, 40.000 mmol). The resulting mixture was stirred at room temperature for 2 hours. The mixture was concentrated under reduced pressure and precipitated with MTBE (10 mL) and heptane (20 mL). The precipitate was washed with cold diethyl ether (15 mL) followed by heptanes (2×25 mL) The solvent was removed under high vacuum to give (2S)-2-amino-2-(2-furyl)ethanol (hydrochloride salt) (1.21 g, 90%) as a white solid; ¹H NMR (400 MHz, DMSO-d6) δ 8.65 (br. s., 3H), 7.71 (d, J=1.0 Hz, 1H), 6.59 (d, J=3.2 Hz, 1H), 6.49 (dd, J=3.2, 1.7 Hz, 1H), 5.61 (br. s., 1H), 4.34 (t, J=5.6 Hz, 1H), 3.90-3.71 (m, 2H). ESI-MS m/z calc. 127.0633, found 128.2 (M+1)⁺; Retention time: 0.21 minutes, LC method I.

### Step 6: (2S)-2-Amino-2-tetrahydrofuran-2-yl-ethanol

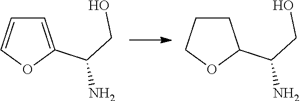 [SMILES: N[C@@H](CO)c1ccco1 ; N[C@@H](CO)C1CCCO1]

To a solution of (2S)-2-amino-2-(2-furyl)ethanol (hydrochloride salt) (1.2 g, 6.9681 mmol) and (2S)-2-amino-2-(2-furyl)ethanol (hydrochloride salt) (265 mg, 1.5388 mmol) in methanol (24 mL) was added palladium hydroxide (20% on carbon with 50% wet) (250 mg, 10% w/w, 0.1780 mmol). The mixture was bubbled with nitrogen. The reaction mixture was purged with hydrogen three times. It was stirred at 45° C. under 100 psi hydrogen for 16 hours. The reaction mixture was filtered on Celite and washed with methanol (25 mL). The filtrate was evaporated under reduced pressure, co-evaporated with MeCN (3 times) and lyophilized to give (2S)-2-amino-2-tetrahydrofuran-2-yl-ethanol (hydrochloride salt) (1.45 g, 94%) as a sticky colorless oil; ¹H NMR (400 MHz, DMSO-d6) δ 8.30-7.87 (m, 3H), 5.35 (t, J=4.9 Hz, 0.5H), 5.29 (t, J=4.4 Hz, 0.5H), 4.01-3.85 (m, 1H), 3.83-3.72 (m, 1H), 3.72-3.57 (m, 2H), 3.57-3.48 (m, 1H), 3.15-3.07 (m, 0.5H), 3.01-2.93 (m, 0.5H), 2.03-1.94 (m, 0.5H), 1.93-1.74 (m, 3H), 1.72-1.59 (m, 0.5H). ESI-MS m/z calc. 131.0946, found 132.3 (M+1)⁺; Retention time: 0.55 minutes. LC method I.

### Step 7: Benzyl N-[(1S)-2-hydroxy-1-tetrahydrofuran-2-yl-ethyl]carbamate, SFC peak 1, isomer A, and benzyl N-[(1S)-2-hydroxy-1-tetrahydrofuran-2-yl-ethyl]carbamate, SFC peak 2, isomer B

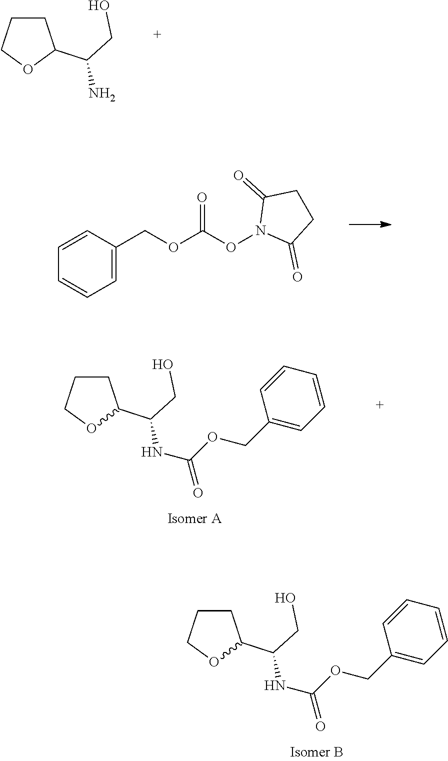 [SMILES: N[C@@H](CO)C1CCCO1 ; O=C(OCc1ccccc1)ON1C(=O)CCC1=O ; O=C(N[C@@H](CO)C1CCCO1)OCc1ccccc1 ; O=C(N[C@@H](CO)C1CCCO1)OCc1ccccc1]

To a stirred solution of (2S)-2-amino-2-tetrahydrofuran-2-yl-ethanol (hydrochloride salt) (1.2 g, 6.6073 mmol) in a mixture of THF (30 mL) and water (30 mL) were added benzyl(2,5-dioxopyrrolidin-1-yl) carbonate (2 g, 8.0251 mmol) and sodium bicarbonate (4 g, 47.615 mmol). The reaction was stirred at room temperature for 3 hours. The reaction was quenched by the addition of a saturated aqueous solution of ammonium chloride (10 mL). The layers were separated, and the aqueous layer was extracted with ethyl acetate (4×25 mL). The combined organic layers were then washed with brine (25 mL), dried over magnesium sulfate, filtered, and concentrated under reduced pressure to afford a colorless oil. The crude was purified by reverse-phase chromatography (acetonitrile in water with 0.1% formic acid). The fractions containing the desired products were combined and concentrated under high vacuum. The resulting mixture of diastereomers was purified by SFC separation using the following conditions: Lux Column 5 μm, Amylose 1 (250×30 mm, 43.7 mg/mL, 21.8 mg/injection), 40° C., eluant: 30% methanol(1% DEA) and 90% CO₂, flow rate: 100 mL/min, injection volume: 500 μL, pressure: 100 bars, wavelength: 210 nm. Evaporation of solvent afforded the two diastereomers as follow: Isomer A, SFC peak 1, benzyl N-[(1S)-2-hydroxy-1-tetrahydrofuran-2-yl-ethyl]carbamate (631 mg, 36%) as a colorless oil; ¹H NMR (400 MHz, CDCl₃) δ 7.42-7.29 (m, 5H), 5.36 (d, J=7.1 Hz, 1H), 5.13 (s, 2H), 4.10-4.01 (m, 1H), 3.93-3.79 (m, 3H), 3.78-3.68 (m, 2H), 2.76 (dd, J=8.7, 2.3 Hz, 1H), 2.05-1.92 (m, 1H), 1.91-1.82 (m, 2H), 1.79-1.67 (m, 1H). ESI-MS m/z calc. 265.1314, found 266.2 (M+1)⁺; Retention time: 1.51 minutes; and isomer B, SFC peak 2, benzyl N-[(1S)-2-hydroxy-1-tetrahydrofuran-2-yl-ethyl]carbamate (700 mg, 40%) as a colorless oil; ¹H NMR (400 MHz, CDCl₃) δ 7.41-7.30 (m, 5H), 5.39 (d, J=6.8 Hz, 1H), 5.12 (s, 2H), 4.00 (q, J=6.8 Hz, 1H), 3.96-3.86 (m, 2H), 3.78-3.64 (m, 3H), 2.73 (br. s., 1H), 2.09-1.98 (m, 1H), 1.98-1.85 (m, 2H), 1.82-1.69 (m, 1H). ESI-MS m/z calc. 265.1314, found 266.2 (M+1)⁺; Retention time: 1.51 minutes. LC method I.

### Step 8: (2S)-2-Amino-2-tetrahydrofuran-2-yl-ethanol, isomer A

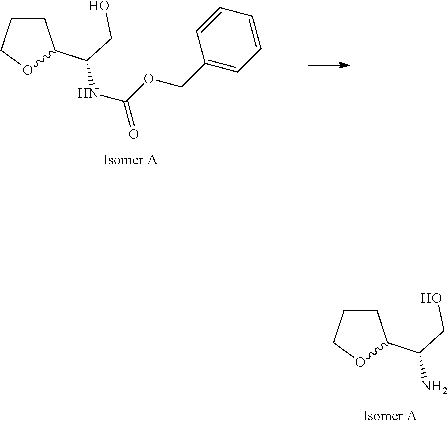 [SMILES: O=C(N[C@@H](CO)C1CCCO1)OCc1ccccc1 ; N[C@@H](CO)C1CCCO1]

A solution of benzyl N-[(1S)-2-hydroxy-1-tetrahydrofuran-2-yl-ethyl]carbamate (630 mg, 2.3723 mmol) (isomer A) in methanol (20 mL) was sparged with nitrogen for 10 minutes then palladium on carbon (255 mg, 10% w/w, 0.2396 mmol) was added. The suspension was bubbled with hydrogen for 10 minutes then stirred under a hydrogen atmosphere for overnight. The suspension was sparged with nitrogen for 10 minutes then was filtered through Celite and over a syringe filter. The Celite pad was washed with MeOH (2×25 mL) and the filtrate was concentrated in vacuo. The mixture was filtered through a Celite pad and the pad was washed with MeOH (20 mL). The combined filtrate was co-evaporated with HCl solution in diethyl ether (2 M, 5 mL). The filtrate was evaporated under reduced pressure, co-evaporated with MeCN (3 times) and lyophilized to give (2S)-2-amino-2-tetrahydrofuran-2-yl-ethanol isomer A (hydrochloride salt) (365 mg, 87%) as a brownish oil; ¹H NMR (400 MHz, DMSO-d6) δ 8.02 (br. s., 3H), 5.33 (br. s., 1H), 3.89 (q, J=7.3 Hz, 1H), 3.81-3.74 (m, 1H), 3.68 (td, J=7.8, 5.9 Hz, 1H), 3.61 (dd, J=11.9, 4.0 Hz, 1H), 3.52 (dd, J=11.7, 6.1 Hz, 1H), 3.02-2.91 (m, 1H), 2.02-1.92 (m, 1H), 1.91-1.78 (m, 2H), 1.71-1.60 (m, 1H). ESI-MS m/z calc. 131.0946, found 132.2 (M+1)⁺; Retention time: 0.54 minutes, LC method J.

### Step 9: 3-[[4-[(2S)-2-Amino-2-tetrahydrofuran-2-yl-ethoxy]-6-(2,6-dimethylphenyl)pyrimidin-2-yl]sulfamoyl]benzoic acid, isomer A

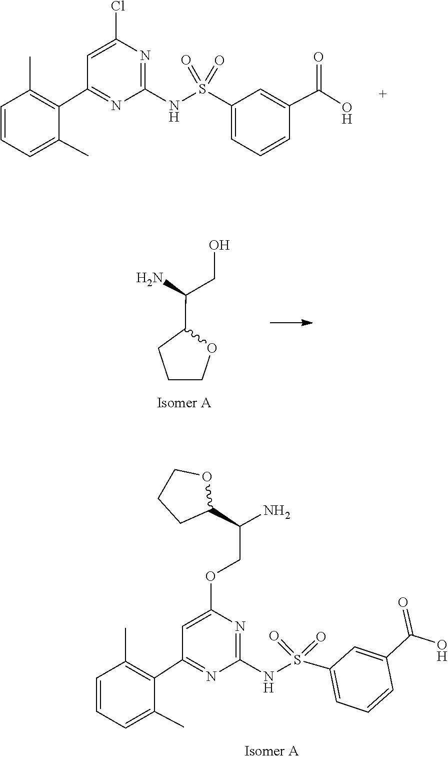 [SMILES: Cc1cccc(C)c1-c1cc(Cl)nc(NS(=O)(=O)c2cccc(C(=O)O)c2)n1 ; N[C@@H](CO)C1CCCO1 ; Cc1cccc(C)c1-c1cc(OC[C@H](N)C2CCCO2)nc(NS(=O)(=O)c2cccc(C(=O)O)c2)n1]

3-[[4-Chloro-6-(2,6-dimethylphenyl)pyrimidin-2-yl]sulfamoyl]benzoic acid (200 mg, 0.4786 mmol) was combined with (2S)-2-amino-2-tetrahydrofuran-2-yl-ethanol isomer A (hydrochloride salt) (approximately 96.27 mg, 0.5743 mmol) in THE (1.3 mL) and stirred for 5 minutes at room temperature. Sodium tert-butoxide (approximately 276.0 mg, 2.872 mmol) was added in one portion. The reaction vessel became warm to the touch and stirring was continued for 1 hour with no external heating. The reaction mixture was then partitioned between 1 M HCl and ethyl acetate. The layers were separated, and an equal volume of brine was added to the aqueous layer, which was then extracted an additional 4×ethyl acetate. The combined organics were washed with brine, dried over sodium sulfate and concentrated to give 3-[[4-[(2S)-2-amino-2-tetrahydrofuran-2-yl-ethoxy]-6-(2,6-dimethylphenyl)pyrimidin-2-yl]sulfamoyl]benzoic acid isomer A (hydrochloride salt) (290.3 mg, 99%). ESI-MS m/z calc. 512.173, found 513.4 (M+1)⁺; Retention time: 0.4 minutes; LC method B.

### Step 10: 3-[[4-[(2S)-2-[(6-tert-Butyl-5-methyl-pyrrolo[2,3-b]pyrazin-3-yl)methylamino]-2-tetrahydrofuran-2-yl-ethoxy]-6-(2,6-dimethylphenyl)pyrimidin-2-yl]sulfamoyl]benzoic acid, isomer A

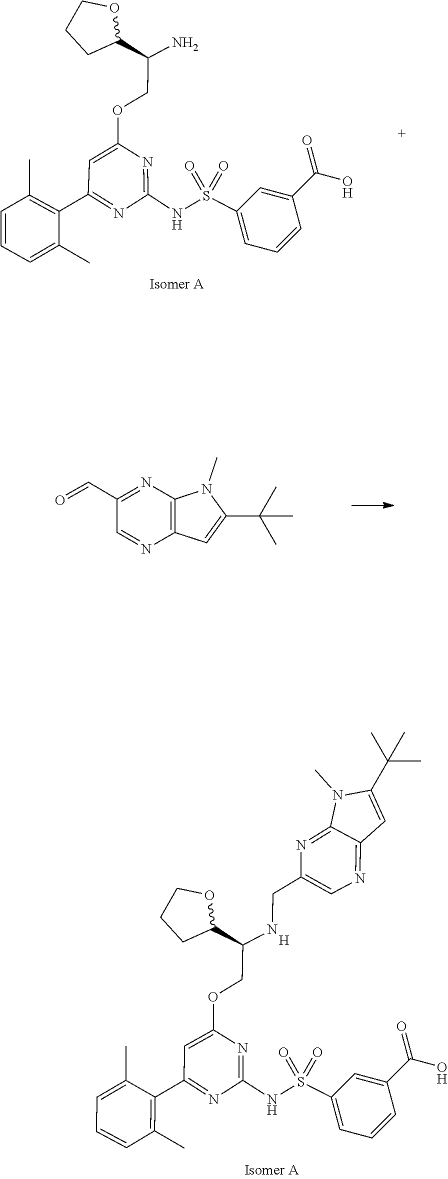

3-[[4-[(2S)-2-Amino-2-tetrahydrofuran-2-yl-ethoxy]-6-(2,6-dimethylphenyl)pyrimidin-2-yl]sulfamoyl]benzoic acid isomer A (hydrochloride salt) (25 mg, 0.04098 mmol) was combined with 6-tert-butyl-5-methyl-pyrrolo[2,3-b]pyrazine-3-carbaldehyde (9.9 mg, 0.04557 mmol) in DCM (0.3 mL) and stirred for 5 minutes at room temperature. AcOH (5 μL, 0.08792 mmol) was added and the reaction was stirred for an additional 10 minutes at room temperature, then was cooled to 0° C. in an ice/water bath. DIPEA (20 μL, 0.1148 mmol) was added and the reaction was stirred for an additional 15 minutes at 0° C. after which sodium triacetoxyborohydride (60 mg, 0.2831 mmol) was added. After 45 minutes at 0° C. the reaction was quenched with 0.3 mL 3M HCl (aq), then diluted with methanol, filtered, and purified by preparative HPLC (1-99% ACN in water, HCl modifier, 15 minute run) to give 3-[[4-[(2S)-2-[(6-tert-butyl-5-methyl-pyrrolo[2,3-b]pyrazin-3-yl)methylamino]-2-tetrahydrofuran-2-yl-ethoxy]-6-(2,6-dimethylphenyl)pyrimidin-2-yl]sulfamoyl]benzoic acid isomer A (hydrochloride salt) (19.8 mg, 64%). ESI-MS m/z calc. 713.29956, found 714.7 (M+1)⁺; Retention time: 0.55 minutes; LC method B.

### Step 11: (11S)-12-[(6-tert-Butyl-5-methyl-pyrrolo[2,3-b]pyrazin-3-yl)methyl]-6-(2,6-dimethylphenyl)-2,2-dioxo-11-tetrahydrofuran-2-yl-9-oxa-2λ⁶-thia-3,5,12,19-tetrazatricyclo[12.3.1.14,8]nonadeca-1(18),4(19),5,7,14,16-hexaen-13-one, isomer A (Compound IV-96

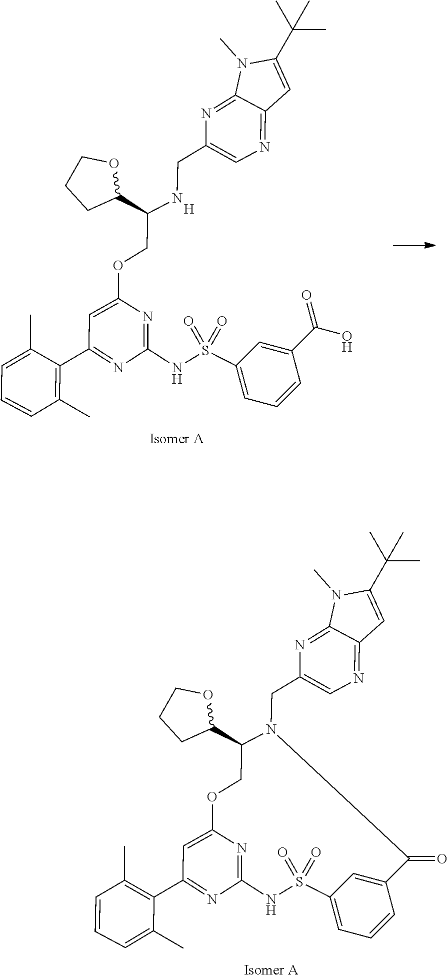

3-[[4-[(2S)-2-[(6-tert-Butyl-5-methyl-pyrrolo[2,3-b]pyrazin-3-yl)methylamino]-2-tetrahydrofuran-2-yl-ethoxy]-6-(2,6-dimethylphenyl)pyrimidin-2-yl]sulfamoyl]benzoic acid isomer A (hydrochloride salt) (19.8 mg, 0.02639 mmol) was combined with CDMT (6 mg, 0.03417 mmol) in DMF (2 mL) at room temperature, and N-methylmorpholine (20 μL, 0.1819 mmol) was added. The reaction was stirred at room temperature for 20 hours then was concentrated by rotary evaporation. After volatiles were removed the resulting residue was dissolved in methanol, filtered, and purified by preparative HPLC (1-99% ACN in water, HCl modifier, 15 minute run) to give (11S)-12-[(6-tert-butyl-5-methyl-pyrrolo[2,3-b]pyrazin-3-yl)methyl]-6-(2,6-dimethylphenyl)-2,2-dioxo-11-tetrahydrofuran-2-yl-9-oxa-2λ⁶-thia-3,5,12,19-tetrazatricyclo[12.3.1.14,8]nonadeca-1(18),4(19),5,7,14,16-hexaen-13-one isomer A (10.1 mg, 53%). ESI-MS m/z calc. 695.289, found 696.7 (M+1)⁺; Retention time: 1.67 minutes; LC method A. ¹H NMR (400 MHz, CDCl₃) δ 8.91 (s, 1H), 8.47 (s, 1H), 8.08 (d, J=7.8 Hz, 1H), 7.88 (d, J=7.4 Hz, 1H), 7.69 (t, J=7.7 Hz, 1H), 7.24 (d, J=7.6 Hz, 1H), 7.10 (d, J=7.6 Hz, 2H), 6.98 (s, 1H), 6.34 (s, 1H), 5.62 (d, J=10.6 Hz, 1H), 5.41 (d, J=15.9 Hz, 1H), 5.10 (d, J=16.0 Hz, 1H), 4.66 (t, J=11.2 Hz, 1H), 4.31-4.24 (m, 1H), 4.22 (s, 3H), 4.09-4.03 (m, 1H), 3.96 (q, J=7.4 Hz, 1H), 3.82 (q, J=7.2 Hz, 1H), 2.09 (s, 6H), 2.02-1.96 (m, 2H), 1.91-1.84 (m, 2H), 1.54 (s, 9H).

### Example 130: Preparation of Compound IV-97

### Step 1: (2S)-2-Amino-2-tetrahydrofuran-2-yl-ethanol isomer B

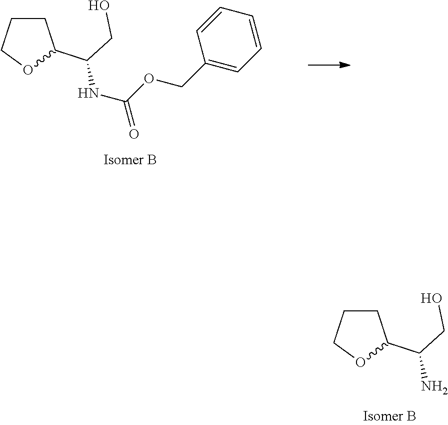 [SMILES: O=C(N[C@@H](CO)C1CCCO1)OCc1ccccc1 ; N[C@@H](CO)C1CCCO1]

A solution of benzyl N-[(1S)-2-hydroxy-1-tetrahydrofuran-2-yl-ethyl]carbamate (700 mg, 2.6358 mmol) (peak 2 of chiral SFC, isomer B) in methanol (20 mL) was sparged with nitrogen for 10 minutes then palladium on carbon (280 mg, 10% w/w, 0.2631 mmol) was added. The suspension was bubbled with hydrogen for 10 minutes then stirred under a hydrogen atmosphere for overnight. The suspension was sparged with nitrogen for 10 minutes then was filtered through Celite and over a syringe filter. The Celite pad was washed with MeOH (2×25 mL) and the filtrate was concentrated in vacuo. The mixture was filtered through a Celite pad and the pad was washed with MeOH (20 mL). The combined filtrate was co-evaporated with HCl solution in diethyl ether (2 M, 5 mL). The filtrate was evaporated under reduced pressure, co-evaporated with MeCN (3 times) and lyophilized to give (2S)-2-amino-2-tetrahydrofuran-2-yl-ethanol isomer B (hydrochloride salt) (440 mg, 95%) as a light brownish oil; ¹H NMR (400 MHz, DMSO-d6) δ 8.13 (br. s., 3H), 5.15 (br. s., 1H), 3.95 (q, J=6.8 Hz, 1H), 3.80-3.73 (m, 1H), 3.69-3.59 (m, 2H), 3.53 (dd, J=11.5, 6.6 Hz, 1H), 3.15-3.05 (m, 1H), 1.97-1.86 (m, 1H), 1.85-1.74 (m, 3H). ESI-MS m/z calc. 131.0946, found 132.3 (M+1)⁺; Retention time: 0.55 minutes. LC method J.

### Step 2: 3-[[4-[(2S)-2-amino-2-tetrahydrofuran-2-yl-ethoxy]-6-(2,6-dimethylphenyl)pyrimidin-2-yl]sulfamoyl]benzoic acid, isomer B

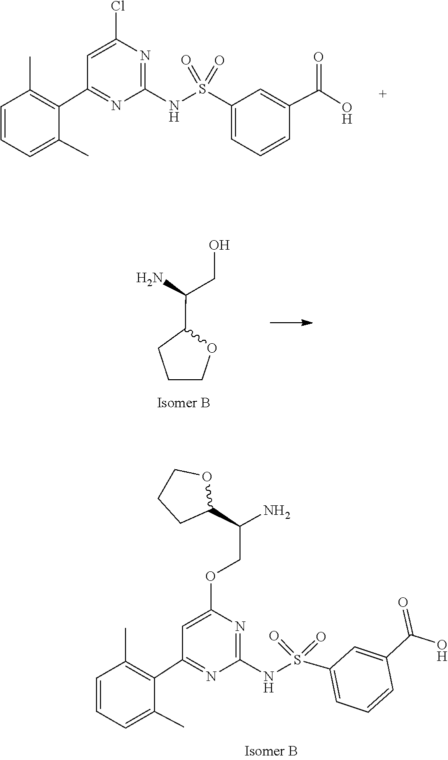 [SMILES: Cc1cccc(C)c1-c1cc(Cl)nc(NS(=O)(=O)c2cccc(C(=O)O)c2)n1 ; N[C@@H](CO)C1CCCO1 ; Cc1cccc(C)c1-c1cc(OC[C@H](N)C2CCCO2)nc(NS(=O)(=O)c2cccc(C(=O)O)c2)n1]

3-[[4-Chloro-6-(2,6-dimethylphenyl)pyrimidin-2-yl]sulfamoyl]benzoic acid (200 mg, 0.4786 mmol) was combined with (2S)-2-amino-2-tetrahydrofuran-2-yl-ethanol; hydrochloride isomer B (approximately 96.27 mg, 0.5743 mmol) in THE and stirred for 5 minutes at room temperature. sodium tert-butoxide (approximately 276.0 mg, 2.872 mmol) was added in one portion. The reaction vessel became warm to the touch and stirring was continued for 1 hour with no external heating. The reaction mixture was then partitioned between 1 M HCl and ethyl acetate. The layers were separated, and an equal volume of brine was added to the aqueous layer, which was then extracted an additional 4×ethyl acetate. The combined organics were washed with brine, dried over sodium sulfate and concentrated to give 3-[[4-[(2S)-2-amino-2-tetrahydrofuran-2-yl-ethoxy]-6-(2,6-dimethylphenyl)pyrimidin-2-yl]sulfamoyl]benzoic acid isomer B (hydrochloride salt) (293.8 mg, 101%). ESI-MS m/z calc. 512.173, found 513.5 (M+1)⁺; Retention time: 0.39 minutes; LC method B

### Step 3: 3-[[4-[(2S)-2-[(6-tert-Butyl-5-methyl-pyrrolo[2,3-b]pyrazin-3-yl)methylamino]-2-tetrahydrofuran-2-yl-ethoxy]-6-(2,6-dimethylphenyl)pyrimidin-2-yl]sulfamoyl]benzoic acid, isomer B

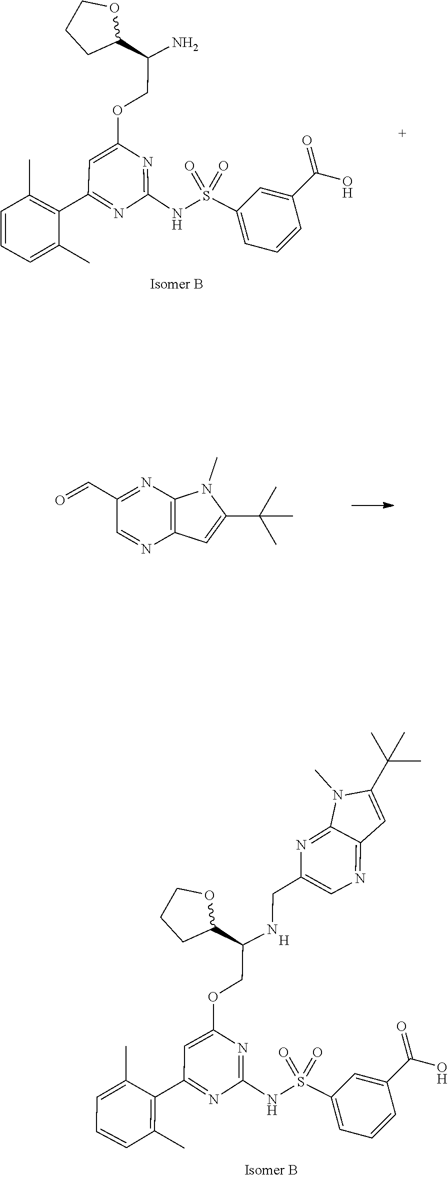

3-[[4-[(2S)-2-amino-2-tetrahydrofuran-2-yl-ethoxy]-6-(2,6-dimethylphenyl)pyrimidin-2-yl]sulfamoyl]benzoic acid isomer B (hydrochloride salt) (25 mg, 0.04098 mmol) was combined with 6-tert-butyl-5-methyl-pyrrolo[2,3-b]pyrazine-3-carbaldehyde (9.9 mg, 0.04557 mmol) in DCM (0.3 mL) and stirred for 5 minutes at room temperature. AcOH (5 μL, 0.08792 mmol) was added and the reaction was stirred for an additional 10 minutes at room temperature, then was cooled to 0° C. in an ice/water bath. DIPEA (20 μL, 0.1148 mmol) was added and the reaction was stirred for an additional 15 minutes at 0° C. after which sodium triacetoxyborohydride (60 mg, 0.2831 mmol) was added. After 45 minutes at 0° C. the reaction was quenched with 0.3 mL 3M HCl (aq), then diluted with methanol, filtered, and purified by preparative HPLC (1-99% ACN in water, HCl modifier, 15 minute run) to give 3-[[4-[(2S)-2-[(6-tert-butyl-5-methyl-pyrrolo[2,3-b]pyrazin-3-yl)methylamino]-2-tetrahydrofuran-2-yl-ethoxy]-6-(2,6-dimethylphenyl)pyrimidin-2-yl]sulfamoyl]benzoic acid isomer B (hydrochloride salt) (23.3 mg, 76%). ESI-MS m/z calc. 713.29956, found 714.7 (M+1)⁺; Retention time: 0.55 minutes; LC method B.

### Step 4: (11S)-12-[(6-tert-Butyl-5-methyl-pyrrolo[2,3-b]pyrazin-3-yl)methyl]-6-(2,6-dimethylphenyl)-2,2-dioxo-11-tetrahydrofuran-2-yl-9-oxa-2λ⁶-thia-3,5,12,19-tetrazatricyclo[12.3.1.14,8]nonadeca-1(18),4(19),5,7,14,16-hexaen-13-one, isomer B (Compound IV-97

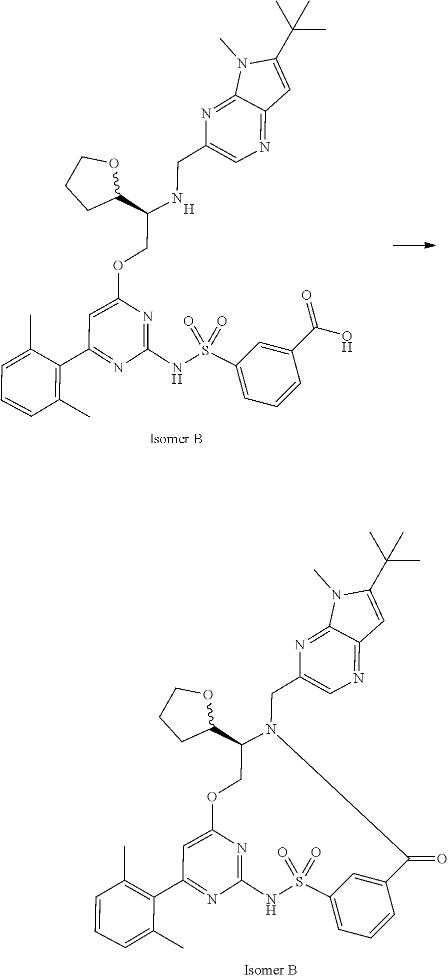

3-[[4-[(2S)-2-[(6-tert-butyl-5-methyl-pyrrolo[2,3-b]pyrazin-3-yl)methylamino]-2-tetrahydrofuran-2-yl-ethoxy]-6-(2,6-dimethylphenyl)pyrimidin-2-yl]sulfamoyl]benzoic acid isomer B (hydrochloride salt) (23.3 mg, 0.03105 mmol) was combined with CDMT (7 mg, 0.03987 mmol) in DMF (2 mL) at room temperature, and N-methylmorpholine (25 μL, 0.2274 mmol) was added. The reaction was stirred at room temperature for 20 hours then was concentrated by rotary evaporation. After volatiles were removed the resulting residue was dissolved in methanol, filtered, and purified by preparative HPLC (1-99% ACN in water, HCl modifier, 15 minute run) to give (11S)-12-[(6-tert-butyl-5-methyl-pyrrolo[2,3-b]pyrazin-3-yl)methyl]-6-(2,6-dimethylphenyl)-2,2-dioxo-11-tetrahydrofuran-2-yl-9-oxa-2λ⁶-thia-3,5,12,19-tetrazatricyclo[12.3.1.14,8]nonadeca-1(18),4(19),5,7,14,16-hexaen-13-one (12.2 mg, 55%). ESI-MS m/z calc. 695.289, found 696.7 (M+1)⁺; Retention time: 1.67 minutes; LC method A. ¹H NMR (400 MHz, CDCl₃) δ 8.92 (s, 1H), 8.48 (s, 1H), 8.12 (d, J=7.8 Hz, 1H), 7.89 (d, J=7.5 Hz, 1H), 7.70 (t, J=7.7 Hz, 1H), 7.23 (d, J=7.7 Hz, 1H), 7.09 (d, J=7.7 Hz, 2H), 6.95 (s, 1H), 6.26 (s, 1H), 5.61-5.43 (m, 2H), 4.86 (d, J=15.1 Hz, 1H), 4.73-4.64 (m, 1H), 4.28 (d, J=10.3 Hz, 1H), 4.20 (s, 4H), 3.88-3.74 (m, 2H), 2.08 (s, 6H), 1.83-1.78 (m, 2H), 1.55 (s, 9H), 1.34-1.26 (m, 2H).

### Example 131: Preparation of Compound IV-98

### Step 1: (5R)-5-(Hydroxymethyl)tetrahydrofuran-2-one

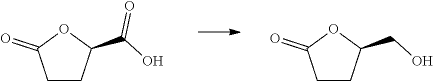 [SMILES: O=C1CC[C@H](C(=O)O)O1 ; O=C1CC[C@H](CO)O1]

To a solution of (2R)-5-oxotetrahydrofuran-2-carboxylic acid (4 g, 29.209 mmol) in anhydrous THE (28 mL) at −78° C. was added dropwise under nitrogen a solution of borane tetrahydrofuran complex (in THF) (44 mL of 1 M, 44.000 mmol). The solution was slowly allowed to warm to room temperature in the dry-ice bath and stirred for 16 hours. The reaction was cooled to 0° C. and MeOH (30 mL) was slowly added dropwise. The solution was evaporated under reduced pressure and the crude material was purified by silica gel chromatography (Gradient: 0-50% EtOAc in DCM). Fractions containing the desired product were combined and repurified by silica gel chromatography (Gradient: 0-100% EtOAc in heptane) to afford (5R)-5-(hydroxymethyl)tetrahydrofuran-2-one (2.69 g, 75%) as an orange oil. H NMR (400 MHz, CDCl₃) δ 4.64 (tdd, J=7.3, 4.6, 2.8 Hz, 1H), 3.92 (dd, J=12.5, 2.9 Hz, 1H), 3.66 (dd, J=12.5, 4.6 Hz, 1H), 2.70-2.50 (m, 2H), 2.47-2.33 (m, 1H), 2.32-2.10 (m, 2H). ESI-MS m/z calc. 116.0473, found 117.2 (M+1)⁺; Retention time: 0.26 minutes. LC method I.

### Step 2: (5R)-5-(Tetrahydropyran-2-yloxymethyl)tetrahydrofuran-2-one

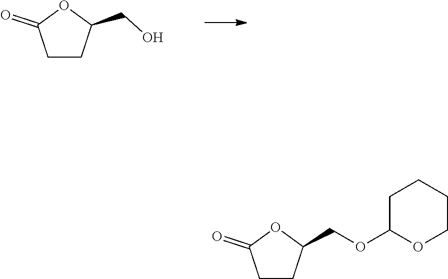 [SMILES: O=C1CC[C@H](CO)O1 ; O=C1CC[C@H](COC2CCCCO2)O1]

To a solution of (5R)-5-(hydroxymethyl)tetrahydrofuran-2-one (6.93 g, 59.682 mmol) in anhydrous DCM (48 mL) was added at room temperature 3,4-dihydro-2H-pyran (5.9930 g, 6.5 mL, 71.247 mmol) followed by pyridinium p-toluene-sulfonate (1.58 g, 6.1615 mmol). The reaction mixture was stirred for 16 hours at this temperature. The reaction was then diluted in DCM (100 mL), quenched with water (100 mL) and the phases were separated. The aqueous phase was extracted with DCM (3×100 mL). The combined organic layers were washed with brine (100 mL), dried over anhydrous sodium sulfate, filtered, and concentrated under reduced pressure. The crude product was purified by silica gel chromatography (Gradient: 0-50% EtOAc in heptane) to provide a mixture of diastereomers (3:1) of (5R)-5-(tetrahydropyran-2-yloxymethyl)tetrahydrofuran-2-one (11.35 g, 95%) as a light yellow oil. ESI-MS m/z calc. 200.1049, found 201.2 (M+1)⁺; Retention time: 1.42 minutes. LC method I.

### Step 3: (2R)-5-Methyl-1-tetrahydropyran-2-yloxy-hexane-2,5-diol

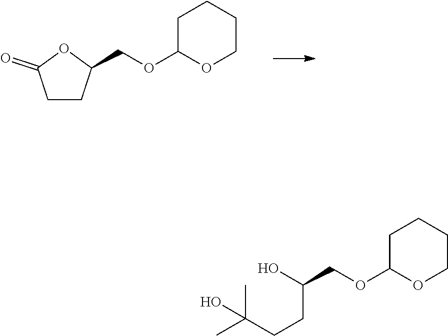 [SMILES: O=C1CC[C@H](COC2CCCCO2)O1 ; CC(C)(O)CC[C@@H](O)COC1CCCCO1]

To a solution of (5R)-5-(tetrahydropyran-2-yloxymethyl)tetrahydrofuran-2-one (11.35 g, 56.628 mmol) in anhydrous THE (87 mL) at 0° C. was added dropwise a solution of methyl magnesium bromide in THE (43 mL of 3 M, 129.00 mmol). The reaction mixture was stirred at 0° C. for 4 hours, then was allowed to warm up to room temperature and stirred for 16 hours. The reaction was quenched with a saturated aqueous solution of ammonium chloride (200 mL), the phases were separated, and the aqueous phase was extracted with EtOAc (3×150 mL). The combined organic layers were washed with brine (150 mL), dried over anhydrous sodium sulfate, filtered, and concentrated under reduced pressure to afford a mixture of diastereomers (2.7:1.0) of the crude (2R)-5-methyl-1-tetrahydropyran-2-yloxy-hexane-2,5-diol (12.75 g, 92%) as a light-yellow oil. ESI-MS m/z calc. 232.1675, no ionization, Retention time: 1.37 minutes; LC method I.

### Step 4: [(2R)-5,5-Dimethyltetrahydrofuran-2-yl]methanol

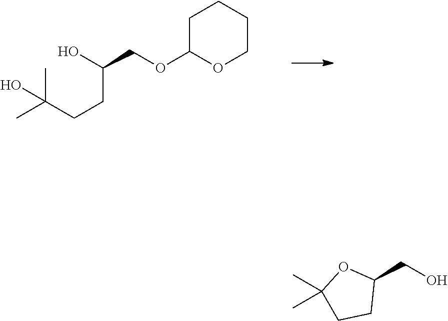

(2R)-5-Methyl-1-tetrahydropyran-2-yloxy-hexane-2,5-diol (12.75 g, 52.138 mmol) was dissolved in methanol (130 mL) and HCl aqueous solution (63.5 mL of 3 M, 190.50 mmol) was added at room temperature. The mixture was heated at 80° C. in a sealed tube for 16 hours, then cooled at room temperature. Solid sodium bicarbonate was added until pH=7 was obtained. The mixture was evaporated under reduced pressure until most of the MeOH was removed, then dissolved in water (150 mL). The aqueous phase was extracted with Me-THF (3×150 mL). The organic layers were combined, washed with brine (150 mL), dried over anhydrous sodium sulfate, filtered and concentrated under reduced pressure to provide the crude [(2R)-5,5-dimethyltetrahydrofuran-2-yl]methanol (8.23 g, 97%) as an orange liquid. ¹H NMR (400 MHz, CDCl₃) δ 4.15-4.07 (m, 1H), 3.69 (dd, J=11.5, 3.2 Hz, 1H), 3.49 (dd, J=11.4, 5.5 Hz, 1H), 2.03-1.93 (m, 1H), 1.90-1.61 (m, 4H overlapped with water), 1.28 (s, 3H), 1.26 (s, 3H). ESI-MS m/z calc. 130.0994, found 131.2 (M+1)⁺; Retention time: 1.6 minutes. LC method I.

### Step 5: (2R)-5,5-Dimethyltetrahydrofuran-2-carbaldehyde

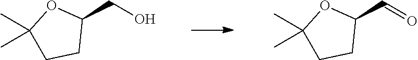

To a solution of [(2R)-5,5-dimethyltetrahydrofuran-2-yl]methanol (8.23 g, 50.574 mmol) in DCM (200 mL) were added successively water (60 mL), sodium bromide (560 mg, 5.4425 mmol), sodium bicarbonate (6.60 g, 78.565 mmol) and TEMPO (250 mg, 1.6001 mmol). The mixture was cooled down to 0° C. and a NaOCl aqueous solution (60 mL of 0.9 M, 54.000 mmol) was added dropwise. The mixture was left stirring at the same temperature for 3 hours, then an NaOCl aqueous solution (60 mL of 0.9 M, 54.000 mmol) was added dropwise. The mixture was stirred for 1 hour, then was filtered over celite, before the phases were separated. The aqueous phase was extracted with DCM (2×100 mL). The combined organic phases were washed with brine (100 mL), dried with anhydrous sodium sulfate filtered and concentrated under reduced pressure (300 mbar, 30° C.) to afford (2R)-5,5-dimethyltetrahydrofuran-2-carbaldehyde as a solution in DCM. The crude solution was directly engaged in the following step.

### Step 6: Methyl (Z)-2-(tert-butoxycarbonylamino)-3-[(2R)-5,5-dimethyltetrahydrofuran-2-yl]prop-2-enoate

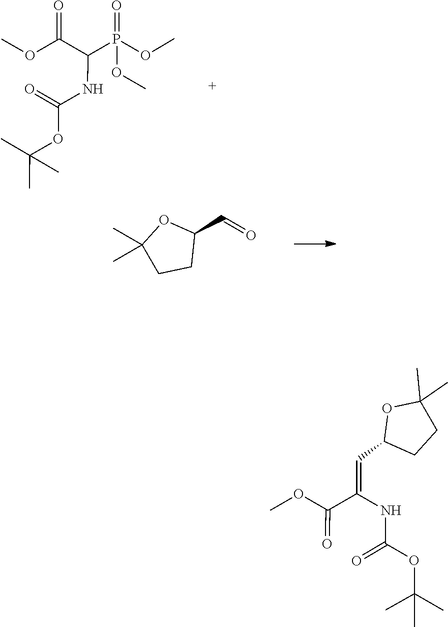

To a stirred solution of (2R)-5,5-dimethyltetrahydrofuran-2-carbaldehyde (estimated 6.48 g, 50.558 mmol) in DCM (66 mL) at 0° C. was added methyl 2-(tert-butoxycarbonylamino)-2-dimethoxyphosphoryl-acetate (15.6 g, 52.482 mmol) followed by DBU (23.414 g, 23 mL, 153.80 mmol). The reaction mixture was stirred at 0° C. for 1 hour and then at room temperature for 16 hours. Water (100 mL) and DCM (50 mL) were added, and the mixture was extracted with DCM (3×100 mL). The combined organic layers were washed with water (2×75 mL), brine (100 mL), dried over anhydrous sodium sulfate, filtered, and concentrated in vacuo. The crude mixture was purified twice by silica gel chromatography (Gradient: 0-10% of EtOAc in heptane) to afford methyl (Z)-2-(tert-butoxycarbonylamino)-3-[(2R)-5,5-dimethyltetrahydrofuran-2-yl]prop-2-enoate (602 mg, 3%) as a colorless oil. ESI-MS m/z calc. 299.1733, found 200.2 (M-99)+; Retention time: 1.8 minutes. LC method I.

### Step 7: Methyl 2-(tert-butoxycarbonylamino)-3-[(2R)-5,5-dimethyltetrahydrofuran-2-yl]propanoate

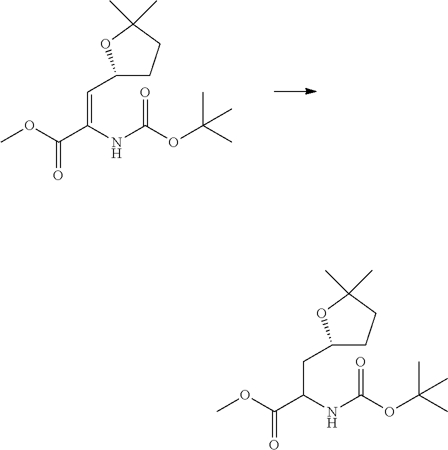 [SMILES: COC(=O)/C(=C/[C@H]1CCC(C)(C)O1)NC(=O)OC(C)(C)C ; COC(=O)C(C[C@H]1CCC(C)(C)O1)NC(=O)OC(C)(C)C]

Nitrogen was bubbled to a solution of methyl (Z)-2-(tert-butoxycarbonylamino)-3-[(2R)-5,5-dimethyltetrahydrofuran-2-yl]prop-2-enoate (602 mg, 1.6128 mmol) in methanol (13 mL) for 15 minutes. Then 1,2-bis[(2R,5R)-2,5-diethylphospholano]benzene(1,5-cyclooctadiene)rhodium(I) trifluoromethanesulfonate (108 mg, 0.1465 mmol) was added and nitrogen was bubbled again for 10 minutes. The reaction was then purged with hydrogen (3×) and the reaction was stirred overnight at 45° C. under 100 psi of hydrogen. The reaction was then cooled down to room temperature and the system was purged with nitrogen. The volatiles were removed under reduced pressure and the residue was purified by silica gel chromatography (Gradient: 0-30% EtOAc in heptane). The fractions containing the product were combined and the residue was repurified by reverse phase chromatography (Isocratic gradient: 50% MeCN in water with 0.1% formic acid) to afford a mixture of two diastereomers of methyl 2-(tert-butoxycarbonylamino)-3-[(2R)-5,5-dimethyltetrahydrofuran-2-yl]propanoate (389 mg, 80%) as a colorless oil. ESI-MS m/z calc. 301.1889, found 202.4 (M-99)⁺; Retention time: 1.77 minutes and 1.83 min. LC method I.

### Step 8: tert-Butyl N-[(1R)-1-[[(2R)-5,5-dimethyltetrahydrofuran-2-yl]methyl]-2-hydroxy-ethyl]carbamate, and tert-butyl N-[(S)-1-[[(2R)-5,5-dimethyltetrahydrofuran-2-yl]methyl]-2-hydroxy-ethyl]carbamate

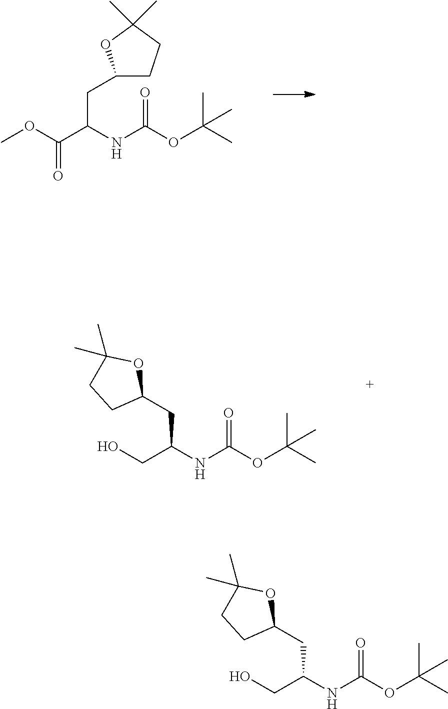 [SMILES: COC(=O)C(C[C@H]1CCC(C)(C)O1)NC(=O)OC(C)(C)C ; CC(C)(C)OC(=O)N[C@@H](CO)C[C@H]1CCC(C)(C)O1 ; CC(C)(C)OC(=O)N[C@H](CO)C[C@H]1CCC(C)(C)O1]

A stirred solution of methyl 2-(tert-butoxycarbonylamino)-3-[(2R)-5,5-dimethyltetrahydrofuran-2-yl]propanoate (389 mg, 1.2894 mmol) in anhydrous THF (8.2 mL) under nitrogen atmosphere and was cooled down to 0° C. Then, a solution of lithium borohydride (in THF) (1.9 mL of 2 M, 3.8000 mmol) was added dropwise. The reaction was stirred at 0° C. for 30 minutes, then allowed to reach room temperature and stirred at room temperature for 16 hours. The reaction was then cooled down to 0° C. and quenched by the dropwise addition of a saturated aqueous solution of ammonium chloride (20 mL). Water (20 mL) and ethyl acetate (20 mL) were then added, and the biphasic mixture was stirred vigorously at 0° C. for 15 minutes. The layers were then separated, and the aqueous layer was extracted with ethyl acetate (3×30 mL). The combined organic layers were washed with brine (30 mL), dried over anhydrous sodium sulfate, filtered, and concentrated under reduced pressure. The crude material was purified by reversed-phase chromatography (Isocratic gradient: 40% MeCN in water with 0.1% formic acid) to afford tert-butyl N-[(1R)-1-[[(2R)-5,5-dimethyltetrahydrofuran-2-yl]methyl]-2-hydroxy-ethyl]carbamate (181 mg, 50%) as a colorless oil. ¹H NMR (400 MHz, CDCl₃) δ 5.37 (br. s., 1H), 4.13-4.04 (m, 1H), 3.81-3.72 (m, 1H), 3.71-3.58 (m, 2H), 2.12-1.97 (m, 2H), 1.83-1.72 (m, 2H), 1.72-1.52 (m, 2H), 1.45 (s, 9H), 1.26 (s, 3H), 1.25 (s, 3H), (1H missing, labile proton). ESI-MS m/z calc. 273.194, found 174.4 (M-99)⁺; Retention time: 1.62 minutes and tert-butyl N-[(1S)-1-[[(2R)-5,5-dimethyltetrahydrofuran-2-yl]methyl]-2-hydroxy-ethyl]carbamate (80.6 mg, 23%) as a colorless oil. ESI-MS m/z calc. 273.194, found 174.4 (M-99)⁺; Retention time: 1.56 minutes.; LC method I.

### Step 9: (2R)-2-Amino-3-[(2R)-5,5-dimethyltetrahydrofuran-2-yl]propan-1-ol

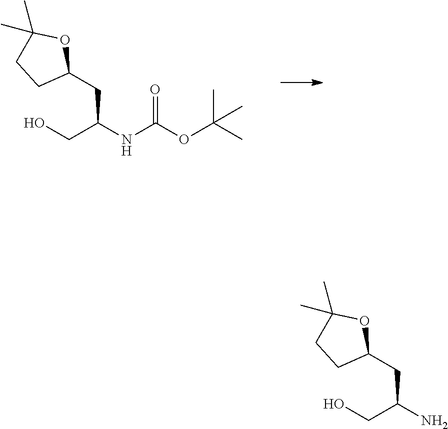 [SMILES: CC(C)(C)OC(=O)N[C@@H](CO)C[C@H]1CCC(C)(C)O1 ; CC1(C)CC[C@H](C[C@@H](N)CO)O1]

To a stirred solution of tert-butyl N-[(1R)-1-[[(2R)-5,5-dimethyltetrahydrofuran-2-yl]methyl]-2-hydroxy-ethyl]carbamate (181 mg, 0.6019 mmol) in tetrahydrofuran (3.5 mL) at 0° C. was added a solution of hydrogen chloride in 1,4-dioxane (3 mL of 4 M, 12.000 mmol). The reaction was stirred at 0° C. for 15 minutes, then the reaction was stirred 20 hours at room temperature. The reaction was then concentrated under reduced pressure, then dried under high vacuum to afford (2R)-2-amino-3-[(2R)-5,5-dimethyltetrahydrofuran-2-yl]propan-1-ol (hydrochloride salt) (156 mg, 100%) as a yellow oil which was directly engaged in the next step without further purification. ¹H NMR (400 MHz, DMSO-d6) δ 7.91 (br. s., 3H), 5.29 (br. s., 1H), 4.04-3.93 (m, 1H), 3.65-3.58 (m, 1H), 3.47 (dd, J=11.5, 6.1 Hz, 1H), 3.19-3.07 (m, 1H), 2.11-1.99 (m, 1H), 1.78-1.57 (m, 4H), 1.57-1.48 (m, 1H), 1.19 (s, 3H), 1.15 (s, 3H).

### Step 10: (2R)-2-Amino-3-[(2R)-5,5-dimethyltetrahydrofuran-2-yl]propan-1-ol

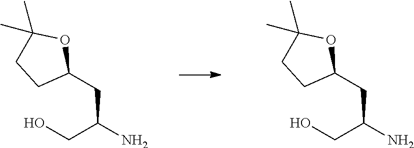

Two batches of (2R)-2-amino-3-[(2R)-5,5-dimethyltetrahydrofuran-2-yl]propan-1-ol (hydrochloride salt) (64 mg, 0.2899 mmol) and (2R)-2-amino-3-[(2R)-5,5-dimethyltetrahydrofuran-2-yl]propan-1-ol (hydrochloride salt) (156 mg, 0.6025 mmol) were dissolved in MeCN (5 mL) and transferred to a vial. The solvent was evaporated under reduced pressure to afford (2R)-2-amino-3-[(2R)-5,5-dimethyltetrahydrofuran-2-yl]propan-1-ol (hydrochloride salt) (194 mg, 99%) ¹H NMR (400 MHz, DMSO-d6) δ 7.83 (br. s., 3H), 5.34-5.22 (m, 1H), 4.04-3.92 (m, 1H), 3.65-3.58 (m, 1H), 3.52-3.42 (m, 1H), 3.21-3.08 (m, 1H), 2.10-1.99 (m, 1H), 1.77-1.49 (m, 5H), 1.19 (s, 3H), 1.15 (s, 3H). ESI-MS m/z calc. 173.1416, found 174.2 (M+1)⁺; Retention time: 0.88 minutes as a yellow solid. LC method I.

### Step 11: 3-[[4-[(2R)-2-Amino-3-[(2R)-5,5-dimethyltetrahydrofuran-2-yl]propoxy]-6-(2,6-dimethylphenyl)pyrimidin-2-yl]sulfamoyl]benzoic acid

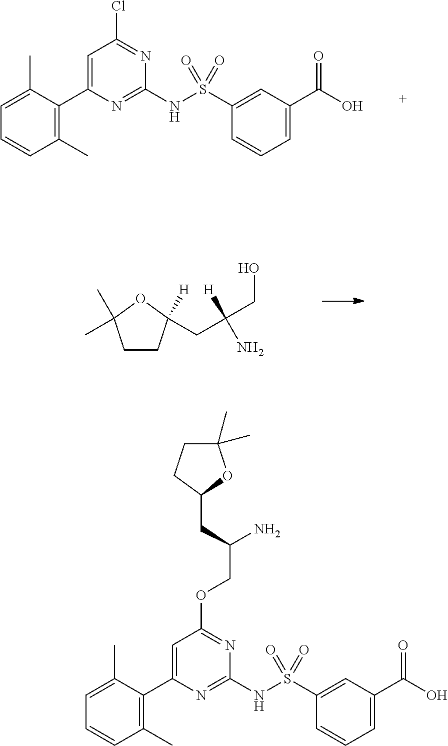 [SMILES: Cc1cccc(C)c1-c1cc(Cl)nc(NS(=O)(=O)c2cccc(C(=O)O)c2)n1 ; CC1(C)CC[C@H](C[C@@H](N)CO)O1 ; Cc1cccc(C)c1-c1cc(OC[C@H](N)C[C@H]2CCC(C)(C)O2)nc(NS(=O)(=O)c2cccc(C(=O)O)c2)n1]

Both 3-[[4-chloro-6-(2,6-dimethylphenyl)pyrimidin-2-yl]sulfamoyl]benzoic acid (200 mg, 0.4786 mmol) and (2R)-2-amino-3-[(2R)-5,5-dimethyltetrahydrofuran-2-yl]propan-1-ol (hydrochloride salt) (100 mg, 0.4768 mmol) were combined in THE (3 mL). At room temperature, sodium tert-butoxide (276 mg, 2.872 mmol) was added in one portion. The reaction mixture was stirred for 1 hour. The reaction mixture was cooled to 0° C. before being quenched with the addition of aqueous HCl (1 μM). It was mixed with EtOAc (50 mL) and HCl (1 M, 50 mL). The aqueous layer was extracted with EtOAc (50 mL). All organic layers were combined, washed with brine (1×50 mL), dried over sodium sulfate filtered and concentrated under reduced pressure. The obtained solid was triturated with EtOAc and hexanes. Solids were collected by vacuum filtration, rinsing with hexanes. This provided 3-[[4-[(2R)-2-amino-3-[(2R)-5,5-dimethyltetrahydrofuran-2-yl]propoxy]-6-(2,6-dimethylphenyl)pyrimidin-2-yl]sulfamoyl]benzoic acid (hydrochloride salt) (270 mg, 95%) as a white solid. ESI-MS m/z calc. 554.2199, found 555.3 (M+1)⁺; Retention time: 1.18 minutes. LC method A.

### Step 12: 3-[[4-[(2R)-2-[(6-tert-Butyl-5-methyl-pyrrolo[2,3-b]pyrazin-3-yl)methylamino]-3-[(2R)-5,5-dimethyltetrahydrofuran-2-yl]propoxy]-6-(2,6-dimethylphenyl)pyrimidin-2-yl]sulfamoyl]benzoic acid

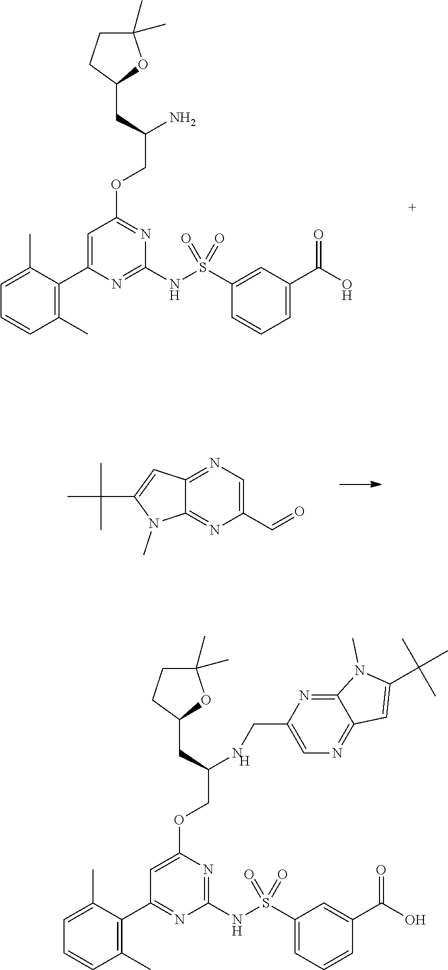

Both 3-[[4-[(2R)-2-amino-3-[(2R)-5,5-dimethyltetrahydrofuran-2-yl]propoxy]-6-(2,6-dimethylphenyl)pyrimidin-2-yl]sulfamoyl]benzoic acid (hydrochloride salt) (17.73 mg, 0.03 mmol) and 6-tert-butyl-5-methyl-pyrrolo[2,3-b]pyrazine-3-carbaldehyde (approximately 7.170 mg, 0.03300 mmol) were combined in DCM. Glacial acetic acid (approximately 3.603 mg, 3.412 μL, 0.06000 mmol) was added. The mixture was cooled to 0° C. before the addition of DIEA (approximately 11.63 mg, 15.67 μL, 0.09000 mmol). After 15 minutes of stirring, sodium triacetoxyborohydride (approximately 31.79 mg, 0.1500 mmol) was added. The reaction mixture was stirred at 0° C. for 1 hour. Purification by reverse phase HPLC (10-99% acetonitrile/5 mM aqueous HCl over 15 minutes) provided 3-[[4-[(2R)-2-[(6-tert-butyl-5-methyl-pyrrolo[2,3-b]pyrazin-3-yl)methylamino]-3-[(2R)-5,5-dimethyltetrahydrofuran-2-yl]propoxy]-6-(2,6-dimethylphenyl)pyrimidin-2-yl]sulfamoyl]benzoic acid (hydrochloride salt) (9 mg, 38%). ESI-MS m/z calc. 755.3465, found 756.3 (M+1)⁺; Retention time: 1.56 minutes; LC method A.

### Step 13: (11R)-12-[(6-tert-Butyl-5-methyl-pyrrolo[2,3-b]pyrazin-3-yl)methyl]-6-(2,6-dimethylphenyl)-11-[[(2R)-5,5-dimethyltetrahydrofuran-2-yl]methyl]-2,2-dioxo-9-oxa-226-thia-3,5,12,19-tetrazatricyclo[12.3.1.14,8]nonadeca-1(18),4(19),5,7,14,16-hexaen-13-one (Compound IV-98

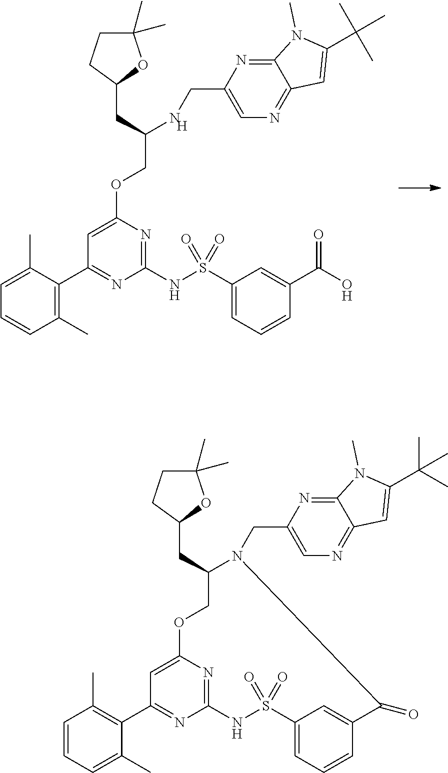

In a 4 mL vial, 3-[[4-[(2R)-2-[(6-tert-butyl-5-methyl-pyrrolo[2,3-b]pyrazin-3-yl)methylamino]-3-[(2R)-5,5-dimethyltetrahydrofuran-2-yl]propoxy]-6-(2,6-dimethylphenyl)pyrimidin-2-yl]sulfamoyl]benzoic acid (hydrochloride salt) (9 mg, 0.01136 mmol) and CDMT (approximately 2.992 mg, 0.01704 mmol) were combined in DMF (0.5 mL). The mixture was cooled to 0° C. before the addition of NMM (approximately 5.745 mg, 6.245 μL, 0.05680 mmol). The reaction mixture was allowed to slowly warm to room temperature and stirred overnight. Purification by reverse phase HPLC (10-99% acetonitrile/5 mM aqueous HCl over 15 minutes) provided (11R)-12-[(6-tert-butyl-5-methyl-pyrrolo[2,3-b]pyrazin-3-yl)methyl]-6-(2,6-dimethylphenyl)-11-[[(2R)-5,5-dimethyltetrahydrofuran-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (4.9 mg, 58%). ESI-MS m/z calc. 737.33594, found 738.3 (M+1)⁺; Retention time: 1.86 minutes; LC method A.

### Example 132: Preparation of Compound IV-102

### Step 1: 6-Chloro-3-(4,4,4-trifluoro-3,3-dimethyl-but-1-ynyl)pyrazin-2-amine

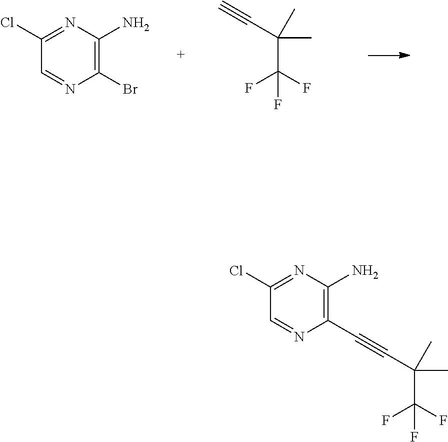

In a flask under nitrogen atmosphere were dissolved 3-bromo-6-chloro-pyrazin-2-amine (963 mg, 4.6199 mmol) and CuI (93 mg, 0.4883 mmol) in THF (20 mL). 4,4,4-Trifluoro-3,3-dimethyl-but-1-yne (691 mg, 5.0766 mmol) and triethylamine (958.32 mg, 1.32 mL, 9.4705 mmol) were then added and the mixture was degassed with nitrogen for 10 min. PdCl₂(PPh₃)₂ (326 mg, 0.4645 mmol) was added and the mixture was re-degassed for another 5 min. The flask was sealed and the reaction was stirred at room temperature for 2 hours. The mixture was filtered through Celite®, washed with EtOAc (30 mL) and the filtrate concentrated in vacuo. Purification was done by reverse-phase chromatography (Gradient: 5-100% MeOH in water with 0.1% formic acid) to give 6-chloro-3-(4,4,4-trifluoro-3,3-dimethyl-but-1-ynyl)pyrazin-2-amine (654 mg, 54%) as an orange solid. ¹H NMR (400 MHz, Chloroform-d) δ 7.89 (s, 1H), 5.10 (br. s., 2H), 1.57 (s, 6H). ¹⁹F NMR (377 MHz, Chloroform-d) 6-77.87 (s, 3F). ESI-MS m/z calc. 263.0437, found 264.2 (M+1)⁺; Retention time: 1.84 minutes. LC method I.

### Step 2: 3-Chloro-6-(2,2,2-trifluoro-1,1-dimethyl-ethyl)-5H-pyrrolo[2,3-b]pyrazine

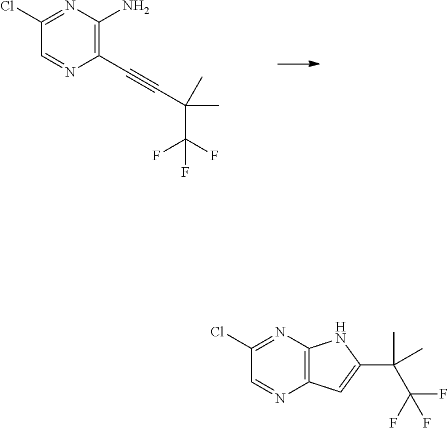 [SMILES: CC(C)(C#Cc1ncc(Cl)nc1N)C(F)(F)F ; CC(C)(c1cc2ncc(Cl)nc2[nH]1)C(F)(F)F]

In a dried-flask under nitrogen atmosphere was dissolved 6-chloro-3-(4,4,4-trifluoro-3,3-dimethyl-but-1-ynyl)pyrazin-2-amine (3.87 g, 14.459 mmol) in dry THE (60 mL). The solution was cooled to 0° C. and potassium tert-butoxide (1M solution in THF) (28 mL of 1 M, 28.000 mmol) was slowly added. The reaction mixture was heated to reflux for 2 hours. The crude was cooled down to room temperature, diluted with EtOAc (20 mL) and quenched with water (15 mL). The aqueous layer was extracted with EtOAc (2×15 mL). The combined organic layers were washed with brine (2×15 mL), dried over sodium sulfate, filtered, and concentrated in vacuo. The crude was purified by reverse-phase chromatography (Gradient: 5-100% MeOH in water with 0.1% formic acid) to afford 3-chloro-6-(2,2,2-trifluoro-1,1-dimethyl-ethyl)-5H-pyrrolo[2,3-b]pyrazine (2.4 g, 32%) ¹H NMR (400 MHz, Chloroform-d) δ 8.78 (br. s., 1H), 8.46 (s, 1H), 6.71 (d, J=2.0 Hz, 1H), 1.66 (s, 6H). ¹⁹F NMR (377 MHz, Chloroform-d) 6-75.83 (s, 3F). ESI-MS m/z calc. 263.0437, found 264.2 (M+1)⁺; Retention time: 1.77 minutes. LC method I.

### Step 3: 3-Chloro-5-methyl-6-(2,2,2-trifluoro-1,1-dimethyl-ethyl)pyrrolo[2,3-b]pyrazine

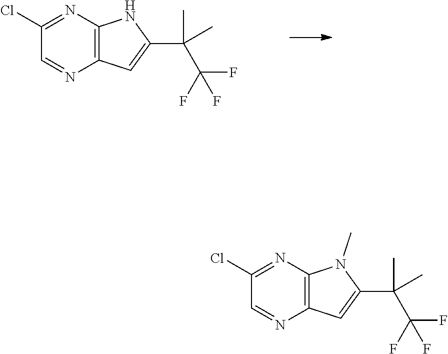 [SMILES: CC(C)(c1cc2ncc(Cl)nc2[nH]1)C(F)(F)F ; Cn1c(C(C)(C)C(F)(F)F)cc2ncc(Cl)nc21]

To a solution of 3-chloro-6-(2,2,2-trifluoro-1,1-dimethyl-ethyl)-5H-pyrrolo[2,3-b]pyrazine (3.55 g, 13.465 mmol) in dry DMF (80 mL) at 0° C. was added NaH (60% dispersion in mineral oil) (1.7 g, 42.504 mmol) portionwise. The mixture was stirred at 0° C. for 10 minutes then dimethyl sulfate (3.4580 g, 2.6 mL, 27.416 mmol) was added dropwise over 20 min. The reaction was stirred at room temperature for 2 hours and cooled to 0° C., water (20 mL) was slowly added followed by MeTHF (20 mL). The aqueous phase was extracted with MeTHF (4×15 mL). The combined organic layers were then washed with brine (2×15 mL), dried over anhydrous sodium sulfate, and filtered. The crude mixture was concentrated in vacuo and was then purified by reverse-phase chromatography (Gradient: 5-100% MeCN in water with 0.1% formic acid) to give 3-chloro-5-methyl-6-(2,2,2-trifluoro-1,1-dimethyl-ethyl)pyrrolo[2,3-b]pyrazine (3 g, 80%) as a tan solid. ¹H NMR (400 MHz, DMSO-d6) δ 8.53 (s, 1H), 6.99 (s, 1H), 3.95 (s, 3H), 1.76 (s, 6H). ¹⁹F NMR (377 MHz, DMSO-d6) δ −73.79 (s, 3F). ESI-MS m/z calc. 277.0594, found 278.1 (M+1)⁺; Retention time: 4.54 minutes. LC method I.

### Step 4: Methyl 5-methyl-6-(2,2,2-trifluoro-1,1-dimethyl-ethyl)pyrrolo[2,3-b]pyrazine-3-carboxylate

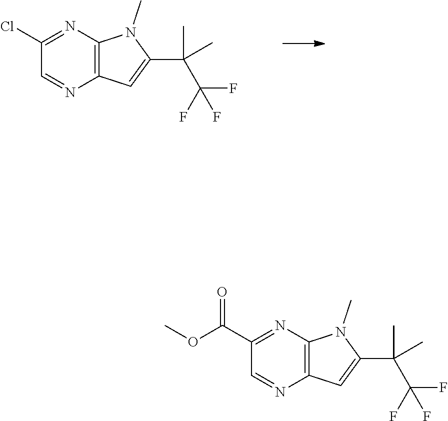 [SMILES: Cn1c(C(C)(C)C(F)(F)F)cc2ncc(Cl)nc21 ; COC(=O)c1cnc2cc(C(C)(C)C(F)(F)F)n(C)c2n1]

To a suspension of 3-chloro-5-methyl-6-(2,2,2-trifluoro-1,1-dimethyl-ethyl)pyrrolo[2,3-b]pyrazine (1.6 g, 5.0707 mmol) in anhydrous methanol (15 mL) was added triethylamine (1.0080 g, 1.4 mL, 9.9615 mmol). Nitrogen was bubbled into the mixture for 15 minutes and then 1,1′-bis(diphenylphosphino)ferrocene palladium(II) chloride, complex with dichloromethane (410 mg, 0.5021 mmol) was added. Nitrogen was bubbled for 5 minutes. The mixture was then stirred at 90° C. under 50 psi of carbon monoxide pressure for 3 hours. The crude was filtered on a Celite pad, washed with EtOAc (15 mL) and concentrated under reduced pressure. Water (15 mL) was added, and the mixture was extracted with EtOAc (3×10 mL). The combined organic layers were washed with brine (10 mL) and dried over anhydrous sodium sulfate, filtered, and concentrated under reduced pressure. The resulting crude was purified by silica gel chromatography (Gradient: 0-70% EtOAc in heptane) to afford methyl 5-methyl-6-(2,2,2-trifluoro-1,1-dimethyl-ethyl)pyrrolo[2,3-b]pyrazine-3-carboxylate (960 mg, 62%) as a light-yellow solid. ESI-MS m/z calc. 301.1038, found 302.2 (M+1)⁺; Retention time: 4.05 minutes. ¹H NMR (400 MHz, DMSO-d6) δ 9.09 (s, 1H), 7.05 (s, 1H), 4.04 (s, 3H), 3.94 (s, 3H), 1.79 (s, 6H). ¹⁹F NMR (377 MHz, DMSO-d6) δ −73.62 (s, 3F). ESI-MS m/z calc. 301.10382, found 302.2 (M+1)⁺; Retention time: 4.05 minutes; LC method J.

### Step 5: [5-Methyl-6-(2,2,2-trifluoro-1,1-dimethyl-ethyl)pyrrolo[2,3-b]pyrazin-3-yl]methanol

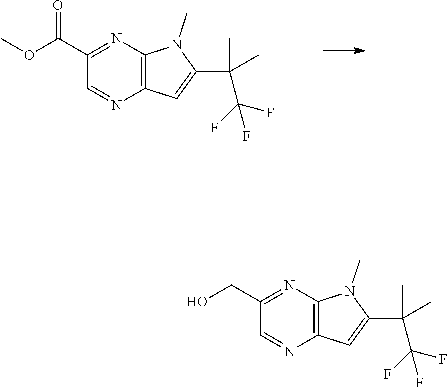 [SMILES: COC(=O)c1cnc2cc(C(C)(C)C(F)(F)F)n(C)c2n1 ; Cn1c(C(C)(C)C(F)(F)F)cc2ncc(CO)nc21]

To a stirred solution of methyl 5-methyl-6-(2,2,2-trifluoro-1,1-dimethyl-ethyl)pyrrolo[2,3-b]pyrazine-3-carboxylate (424 mg, 1.4060 mmol) in DCM (12 mL) at −78° C. was added DIBAL-H (in toluene solution) (3.5 mL of 1 M, 3.5000 mmol) dropwise. The reaction was stirred at that temperature for 1 hour. The reaction was put in a bath at 0° C. and was quenched with water (8 mL) and extracted with dichloromethane (2×10 mL). The organic layer was washed with brine (2×10 mL), dried over anhydrous sodium sulfate, filtered and concentrated in vacuo to give [5-methyl-6-(2,2,2-trifluoro-1,1-dimethyl-ethyl)pyrrolo[2,3-b]pyrazin-3-yl]methanol (333 mg, 80%) as a yellow solid. ¹H NMR (400 MHz, Chloroform-d) δ 8.45 (s, 1H), 6.79 (s, 1H), 4.93 (s, 2H), 4.07 (s, 3H), 1.79 (s, 6H), (1H missing, labile proton). ¹⁹F NMR (377 MHz, Chloroform-d) 6-74.64 (s, 3F). ESI-MS m/z calc. 273.1089, found 274.2 (M+1)⁺; Retention time: 1.57 minutes. LC method I.

### Step 6: 5-Methyl-6-(2,2,2-trifluoro-1,1-dimethyl-ethyl)pyrrolo[2,3-b]pyrazine-3-carbaldehyde

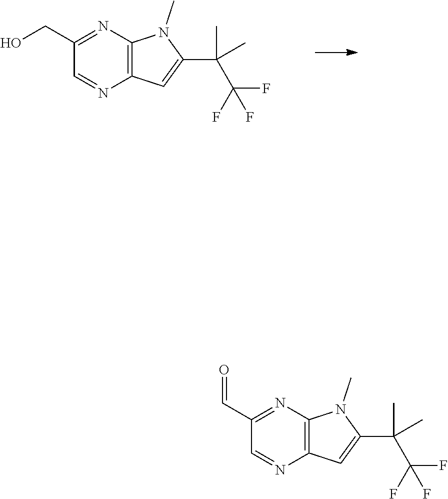 [SMILES: Cn1c(C(C)(C)C(F)(F)F)cc2ncc(CO)nc21 ; Cn1c(C(C)(C)C(F)(F)F)cc2ncc(C=O)nc21]

To a 0° C. solution of [5-methyl-6-(2,2,2-trifluoro-1,1-dimethyl-ethyl)pyrrolo[2,3-b]pyrazin-3-yl]methanol (333 mg, 1.1285 mmol) in dry DCM (12 mL) was added Dess-Martin periodinane (580 mg, 1.3675 mmol) and the solution was stirred at room temperature for 2 hours. To the mixture was added a solution of aqueous saturated sodium thiosulfate (6 mL), saturated sodium bicarbonate (6 mL), water (5 mL) and an aqueous solution of NaOH 1M (4 mL, to reach pH=9). The phases were separated, and the aqueous phase was extracted with DCM (3×10 mL). The combined organic layers were dried over anhydrous sodium sulfate, filtered and concentrated under reduced pressure to afford crude 5-methyl-6-(2,2,2-trifluoro-1,1-dimethyl-ethyl)pyrrolo[2,3-b]pyrazine-3-carbaldehyde (540 mg, 54%) as a yellow solid. ESI-MS m/z calc. 271.0932, found 272.2 (M+1)⁺; Retention time: 1.76 minutes. LC method I. This crude batch of 5-Methyl-6-(2,2,2-trifluoro-1,1-dimethyl-ethyl)pyrrolo[2,3-b]pyrazine-3-carbaldehyde (540 mg, 1.2343 mmol) and another batch of 5-methyl-6-(2,2,2-trifluoro-1,1-dimethyl-ethyl)pyrrolo[2,3-b]pyrazine-3-carbaldehyde (90 mg, 0.1871 mmol) were combined for purification by reverse-phase chromatography (Gradient: 5-100% MeCN in water with 0.1% formic acid) to give 5-methyl-6-(2,2,2-trifluoro-1,1-dimethyl-ethyl)pyrrolo[2,3-b]pyrazine-3-carbaldehyde (250 mg). A second purification by reverse-phase chromatography was performed (Gradient: 5-70% MeCN in water with 0.1% formic acid) to afford 5-methyl-6-(2,2,2-trifluoro-1,1-dimethyl-ethyl)pyrrolo[2,3-b]pyrazine-3-carbaldehyde (124.6 mg, 18%) as a light-yellow solid. ¹H NMR (400 MHz, DMSO-d6) δ 10.13 (s, 1H), 9.01 (s, 1H), 7.11 (s, 1H), 4.08 (s, 3H), 1.81 (s, 6H). ¹⁹F NMR (377 MHz, DMSO-d6) δ −73.60 (s, 3F). ESI-MS m/z calc. 271.0932, found 272.2 (M+1)⁺; Retention time: 4.06 minutes. LC method J.

### Step 7: 3-[[4-[(2R)-3-(1-Bicyclo[1.1.1]pentanyl)-2-[[5-methyl-6-(2,2,2-trifluoro-1,1-dimethyl-ethyl)pyrrolo[2,3-b]pyrazin-3-yl]methylamino]propoxy]-6-(2,6-dimethylphenyl)pyrimidin-2-yl]sulfamoyl]benzoic acid

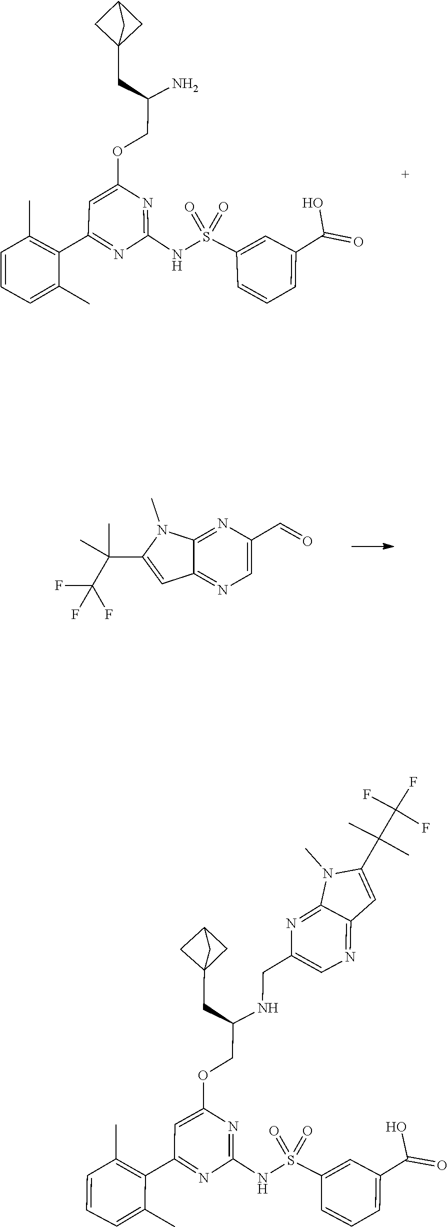 [SMILES: Cc1cccc(C)c1-c1cc(OC[C@H](N)CC23CC(C2)C3)nc(NS(=O)(=O)c2cccc(C(=O)O)c2)n1 ; Cn1c(C(C)(C)C(F)(F)F)cc2ncc(C=O)nc21 ; Cc1cccc(C)c1-c1cc(OC[C@@H](CC23CC(C2)C3)NCc2cnc3cc(C(C)(C)C(F)(F)F)n(C)c3n2)nc(NS(=O)(=O)c2cccc(C(=O)O)c2)n1]

3-[[4-[(2R)-2-Amino-3-(1-bicyclo[1.1.1]pentanyl)propoxy]-6-(2,6-dimethylphenyl)pyrimidin-2-yl]sulfamoyl]benzoic acid (hydrochloride salt) (25 mg, 0.04024 mmol) was combined with 5-methyl-6-(2,2,2-trifluoro-1,1-dimethyl-ethyl)pyrrolo[2,3-b]pyrazine-3-carbaldehyde (12 mg, 0.04424 mmol) in DCM (0.5 mL) and stirred for 10 minutes at room temperature. AcOH (6 μL, 0.1055 mmol) was added and after an additional 10 minutes the reaction mixture was cooled to 0° C. and DIPEA (25 μL, 0.1435 mmol) was added. After 5 minutes sodium triacetoxyborohydride (55 mg, 0.2595 mmol) was added in one portion, and stirring was continued for 2 hours at 0° C. After this time the reaction mixture was quenched with 0.3 mL 3M HCl, diluted with methanol, filtered, and purified by preparative HPLC (1-99% ACN in water, HCl modifier, 15 minute run) to give 3-[[4-[(2R)-3-(1-bicyclo[1.1.1]pentanyl)-2-[[5-methyl-6-(2,2,2-trifluoro-1,1-dimethyl-ethyl)pyrrolo[2,3-b]pyrazin-3-yl]methylamino]propoxy]-6-(2,6-dimethylphenyl)pyrimidin-2-yl]sulfamoyl]benzoic acid (hydrochloride salt) (24 mg, 73%). ESI-MS m/z calc. 777.29205, found 778.7 (M+1)⁺; Retention time: 0.6 minutes; LC method B.

### Step 8: (11R)-11-(1-Bicyclo[1.1.1]pentanylmethyl)-6-(2,6-dimethylphenyl)-12-[[5-methyl-6-(2,2,2-trifluoro-1,1-dimethyl-ethyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound IV-102

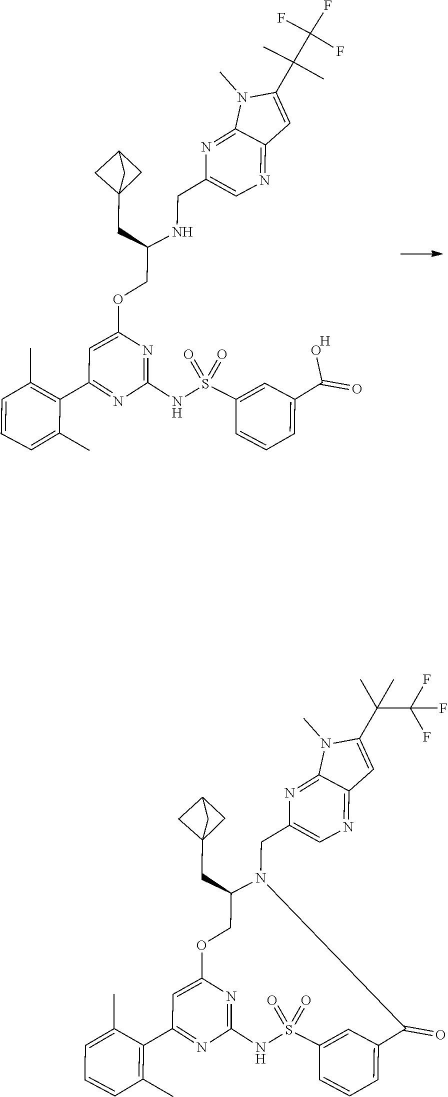

3-[[4-[(2R)-3-(1-Bicyclo[1.1.1]pentanyl)-2-[[5-methyl-6-(2,2,2-trifluoro-1,1-dimethyl-ethyl)pyrrolo[2,3-b]pyrazin-3-yl]methylamino]propoxy]-6-(2,6-dimethylphenyl)pyrimidin-2-yl]sulfamoyl]benzoic acid (hydrochloride salt) (24 mg, 0.02947 mmol) was combined with CDMT (6.5 mg, 0.03702 mmol) in anhydrous DMF (3 mL) in a screwcap vial. N-methylmorpholine (25 μL, 0.2274 mmol) was added and the reaction mixture was stirred for 4 hours at room temperature. Volatiles were then removed by rotary evaporation and the resulting residue was dissolved in methanol, filtered, and purified by preparative HPLC (1-99% ACN in water, HCl modifier, 15 minute run) to give (11R)-11-(1-bicyclo[1.1.1]pentanylmethyl)-6-(2,6-dimethylphenyl)-12-[[5-methyl-6-(2,2,2-trifluoro-1,1-dimethyl-ethyl)pyrrolo[2,3-b]pyrazin-3-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (14.8 mg, 65%). ESI-MS m/z calc. 759.28143, found 760.7 (M+1)⁺; Retention time: 2.05 minutes; LC method A. ¹H NMR (400 MHz, CDCl₃) δ 9.00 (t, J=1.9 Hz, 1H), 8.57 (s, 1H), 8.09 (d, J=7.9 Hz, 1H), 7.92 (dt, J=7.9, 1.4 Hz, 1H), 7.68 (t, J=7.8 Hz, 1H), 7.21 (t, J=7.6 Hz, 1H), 7.07 (d, J=7.6 Hz, 2H), 6.78 (s, 1H), 6.24 (s, 1H), 5.61 (dd, J=11.0, 3.6 Hz, 1H), 5.42 (d, J=15.9 Hz, 1H), 4.32 (d, J=16.0 Hz, 1H), 4.12 (s, 4H), 4.02 (t, J=11.2 Hz, 1H), 2.46 (s, 1H), 2.06 (s, 6H), 1.97 (dd, J=15.5, 10.4 Hz, 1H), 1.84 (dd, J=15.5, 3.3 Hz, 1H), 1.76 (s, 6H), 1.62-1.57 (in, 6H).

### VI. Characterization of Compounds

The compounds in the tables below were prepared by procedures analogous to those disclosed in the specification, and the analytical data were consistent with the reported structure.

### A. LCMS Data

| Compd | | LCMS Rt | Calc. | | LCMS |
|---|---|---|---|---|---|
| No. | Structure | (min) | Mass | M + 1 | Method |
| I-1 | 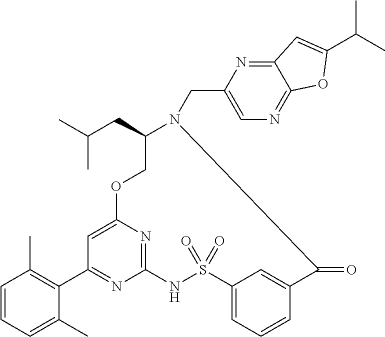 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)C)CO2] | 2.08 | 654.262 | 655.7 | A |
| I-2 | 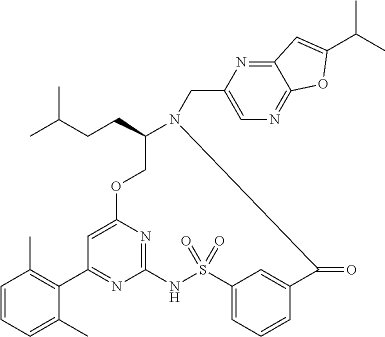 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)C)CO2] | 2.17 | 668.278 | 669.7 | A |
| I-3 | 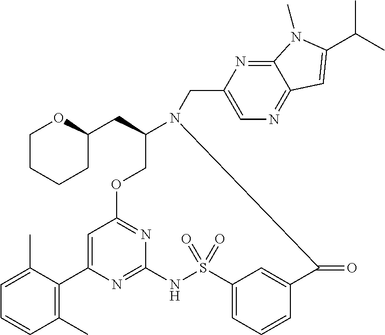 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCCO1)CO2] | 1.7 | 709.305 | 710.65 | A |
| I-4 | 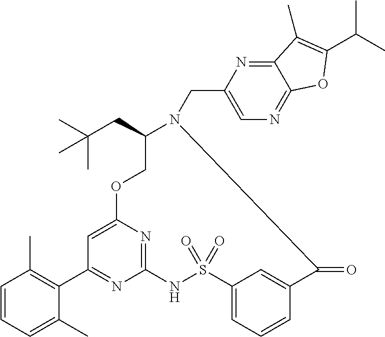 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)C)CO2] | 2.21 | 682.294 | 683.68 | A |
| I-5 | 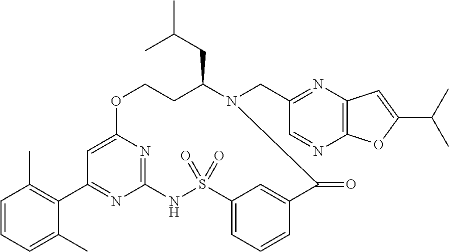 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)C)CCO2] | 2.01 | 668.278 | 669.2 | A |
| I-6 | 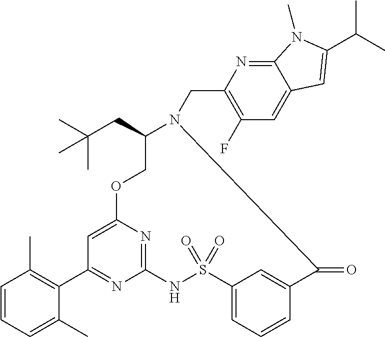 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC(C)(C)C)CO2] | 2.39 | 698.305 | 699.9 | A |
| I-7 | 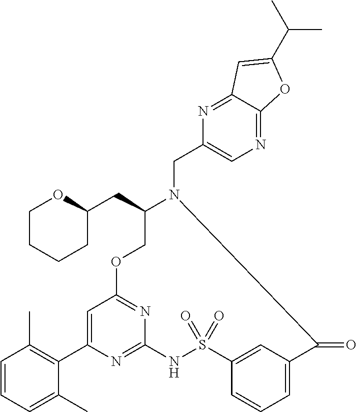 [SMILES: Cc1cccc(C)c1-c1cc2nc(n1)NS(=O)(=O)c1cccc(c1)C(=O)N(Cc1cnc3oc(C(C)C)cc3n1)[C@H](C[C@H]1CCCCO1)CO2] | 1.64 | 696.273 | 697.5 | A |
| I-8 | 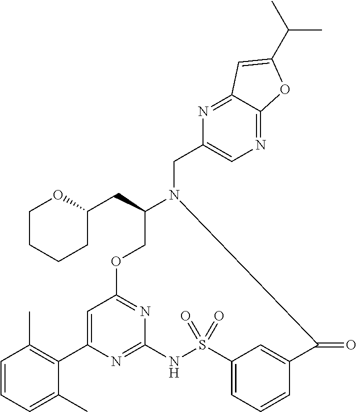 [SMILES: Cc1cccc(C)c1-c1cc2nc(n1)NS(=O)(=O)c1cccc(c1)C(=O)N(Cc1cnc3oc(C(C)C)cc3n1)[C@H](C[C@@H]1CCCCO1)CO2] | 1.66 | 696.273 | 697.5 | A |
| I-9 | 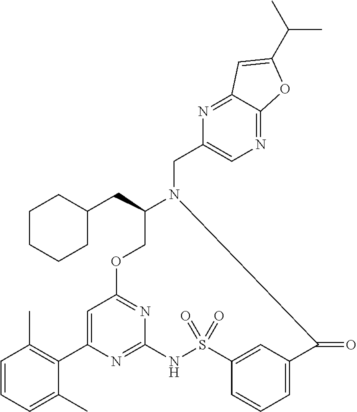 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCCC1)CO2] | 1.88 | 694.294 | 695.5 | A |
| I-10 | 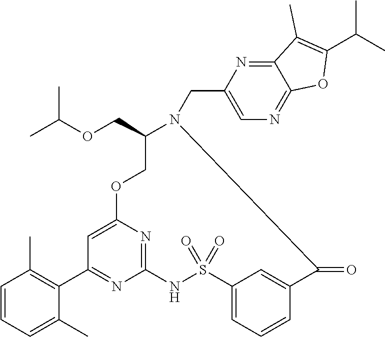 [SMILES: Cc1cccc(C)c1-c1cc2nc(n1)NS(=O)(=O)c1cccc(c1)C(=O)N(Cc1cnc3oc(C(C)C)c(C)c3n1)[C@H](COC(C)C)CO2] | 2.19 | 684.273 | 685.4 | A |
| I-11 | 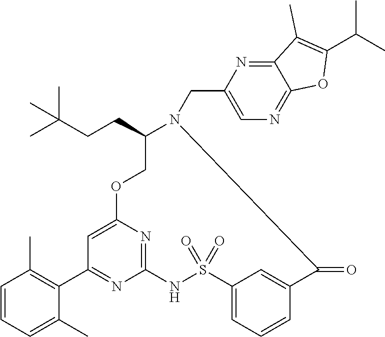 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)(C)C)CO2] | 2.42 | 696.309 | 697.4 | A |
| I-12 | 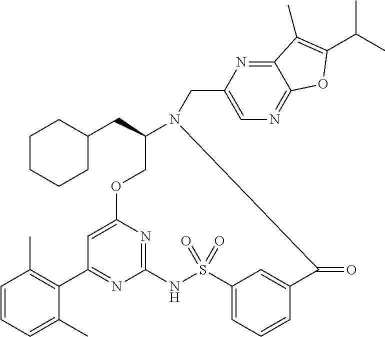 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCCC1)CO2] | 2.44 | 708.309 | 709.3 | A |
| I-14 | 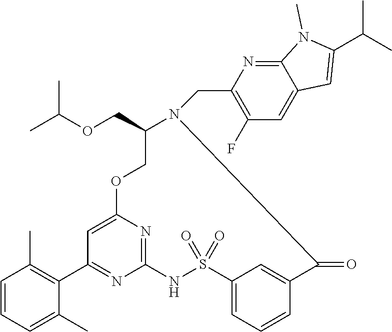 [SMILES: Cc1cccc(C)c1-c1cc2nc(n1)NS(=O)(=O)c1cccc(c1)C(=O)N(Cc1nc3c(cc1F)cc(C(C)C)n3C)[C@H](COC(C)C)CO2] | 2.32 | 700.284 | 701.4 | A |
| I-16 | 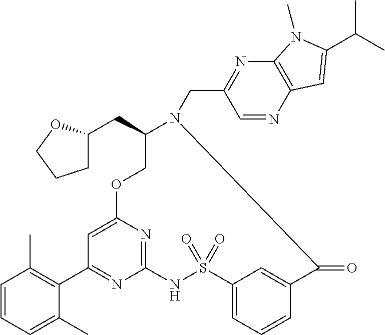 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCO1)CO2] | 1.59 | 695.289 | 695.8 | A |
| I-17 | 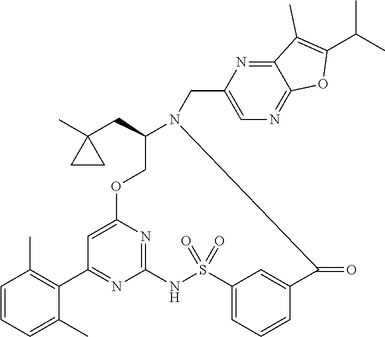 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1(C)CC1)CO2] | 2.15 | 680.278 | 681.92 | A |
| I-19 | 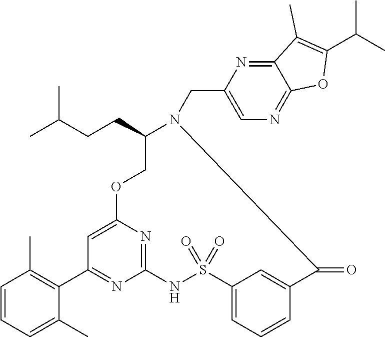 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)C)CO2] | 2.28 | 682.294 | 683.74 | A |
| I-20 | 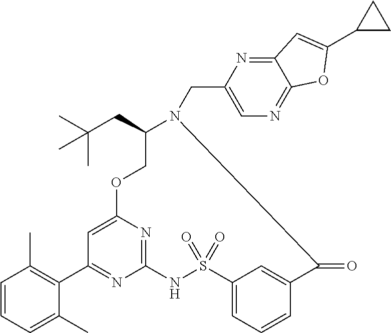 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)C)CO2] | 2.04 | 666.262 | 667.67 | A |
| I-21 | 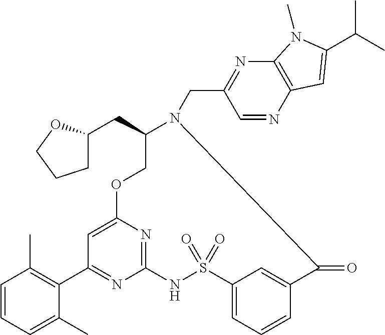 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCO1)CO2] | 1.62 | 695.289 | 696.8 | A |
| I-24 | 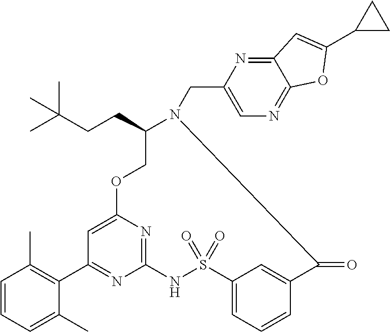 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)C)CO2] | 2.21 | 680.278 | 681.3 | A |
| I-25 | 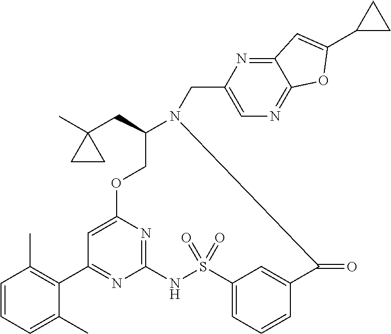 [SMILES: Cc1cccc(C)c1-c1cc2nc(n1)NS(=O)(=O)c1cccc(c1)C(=O)N(Cc1cnc3oc(C4CC4)cc3n1)[C@H](CC1(C)CC1)CO2] | 2.04 | 664.247 | 665.2 | A |
| I-27 | 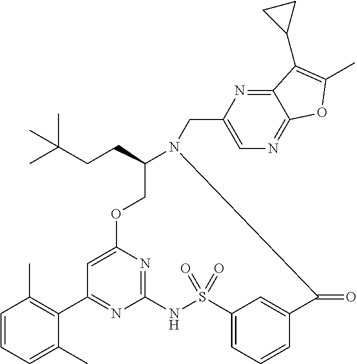 [SMILES: Cc1cccc(C)c1-c1cc2nc(n1)NS(=O)(=O)c1cccc(c1)C(=O)N(Cc1cnc3oc(C)c(C4CC4)c3n1)[C@H](CCC(C)(C)C)CO2] | 2.25 | 694.294 | 695.9 | A |
| I-28 | 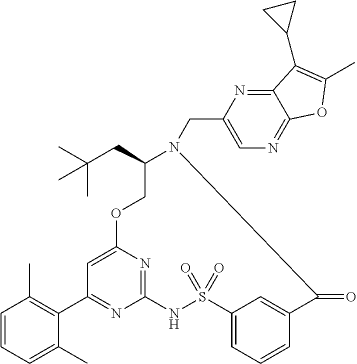 [SMILES: Cc1cccc(C)c1-c1cc2nc(n1)NS(=O)(=O)c1cccc(c1)C(=O)N(Cc1cnc3oc(C)c(C4CC4)c3n1)[C@H](CC(C)(C)C)CO2] | 2.12 | 680.278 | 681.9 | A |
| I-29 | 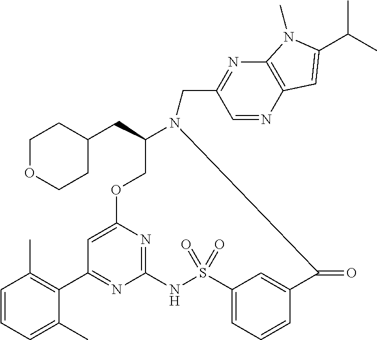 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCOCC1)CO2] | 1.59 | 709.305 | 710.8 | A |
| I-31 | 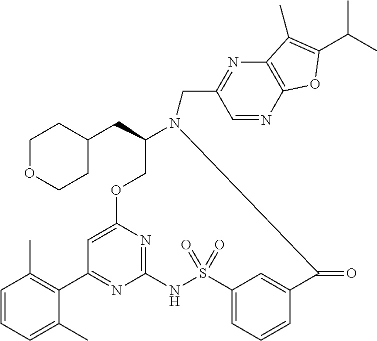 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCOCC1)CO2] | 1.94 | 710.289 | 711.9 | A |
| I-32 | 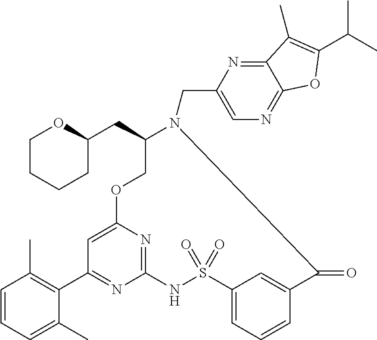 [SMILES: Cc1cccc(C)c1-c1cc2nc(n1)NS(=O)(=O)c1cccc(c1)C(=O)N(Cc1cnc3oc(C(C)C)c(C)c3n1)[C@H](C[C@H]1CCCCO1)CO2] | 2.07 | 710.289 | 711.9 | A |
| I-33 | 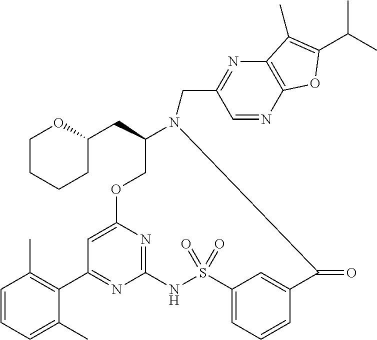 [SMILES: Cc1cccc(C)c1-c1cc2nc(n1)NS(=O)(=O)c1cccc(c1)C(=O)N(Cc1cnc3oc(C(C)C)c(C)c3n1)[C@H](C[C@@H]1CCCCO1)CO2] | 2.11 | 710.289 | 711.8 | A |
| I-34 | 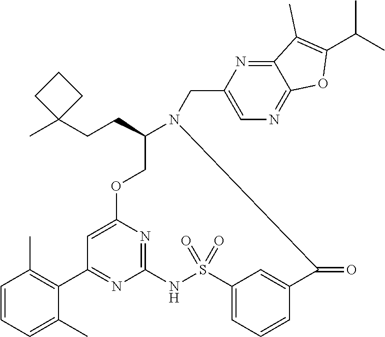 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC1(C)CCC1)CO2] | 2.43 | 708.309 | 709.4 | A |
| I-35 | 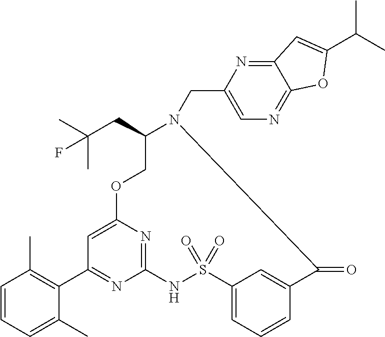 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)F)CO2] | 1.97 | 672.253 | 673.4 | A |
| I-38 | 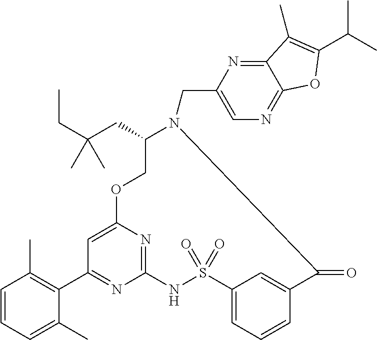 [SMILES: CCC(C)(C)C[C@H]1COc2cc(-c3c(C)cccc3C)nc(n2)NS(=O)(=O)c2cccc(c2)C(=O)N1Cc1cnc2oc(C(C)C)c(C)c2n1] | 2.27 | 682.294 | 683.2 | A |
| I-39 | 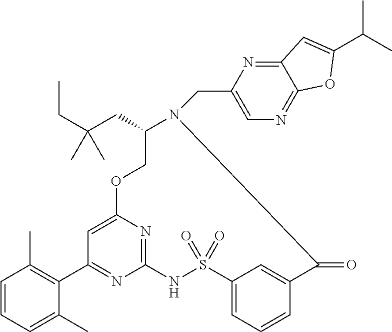 [SMILES: CCC(C)(C)C[C@H]1COc2cc(-c3c(C)cccc3C)nc(n2)NS(=O)(=O)c2cccc(c2)C(=O)N1Cc1cnc2oc(C(C)C)cc2n1] | 2.28 | 682.294 | 683.2 | A |
| I-40 | 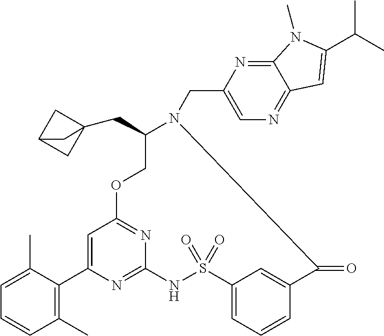 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC13CC(C1)C3)CO2] | 1.83 | 691.294 | 692.7 | A |
| I-41 | 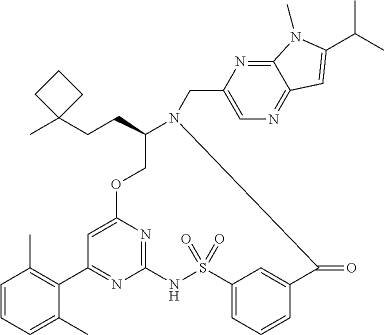 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1(C)CCC1)CO2] | 2.03 | 707.325 | 708.4 | A |
| I-42 | 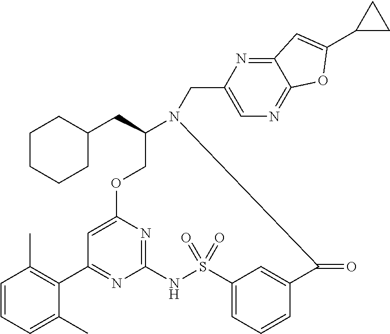 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCCC1)CO2] | 2.21 | 692.278 | 693.2 | A |
| I-43 | 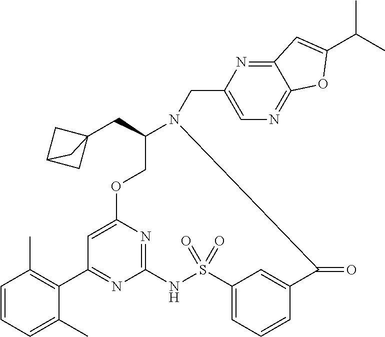 [SMILES: Cc1cccc(C)c1-c1cc2nc(n1)NS(=O)(=O)c1cccc(c1)C(=O)N(Cc1cnc3oc(C(C)C)cc3n1)[C@H](CC13CC(C1)C3)CO2] | 2.22 | 678.262 | 679.2 | A |
| I-44 | 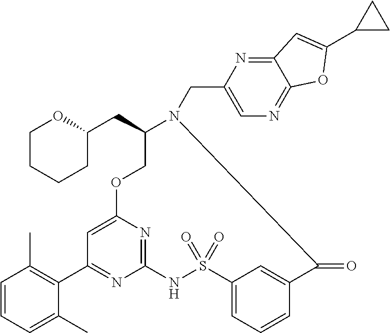 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCCO1)CO2] | 1.86 | 694.257 | 695.9 | A |
| I-45 | 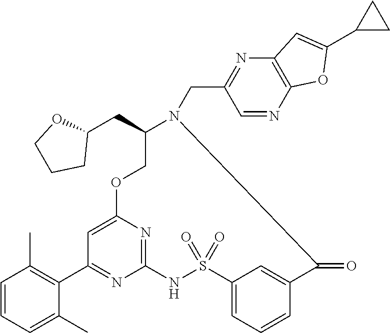 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCO1)CO2] | 1.77 | 680.242 | 681.8 | A |
| I-46 | 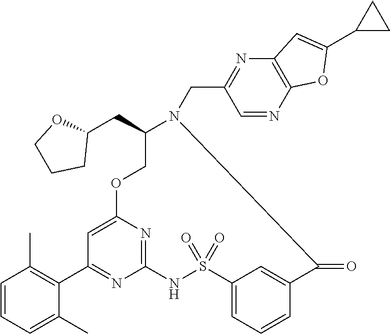 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCO1)CO2] | 1.8 | 680.242 | 681.8 | A |
| I-47 | 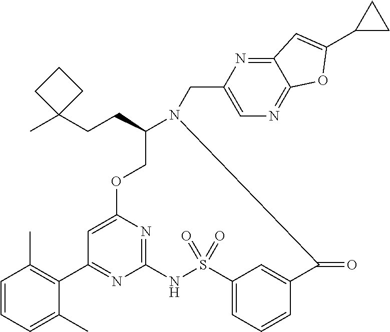 [SMILES: Cc1cccc(C)c1-c1cc2nc(n1)NS(=O)(=O)c1cccc(c1)C(=O)N(Cc1cnc3oc(C4CC4)cc3n1)[C@H](CCC1(C)CCC1)CO2] | 2.19 | 692.278 | 693.9 | A |
| I-48 | 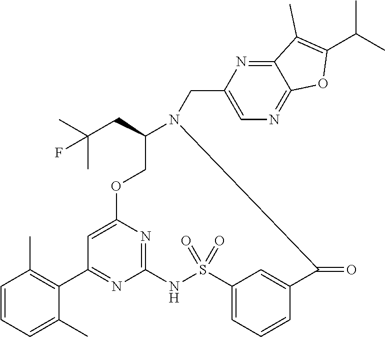 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)F)CO2] | 1.99 | 686.269 | 687.9 | A |
| I-51 | 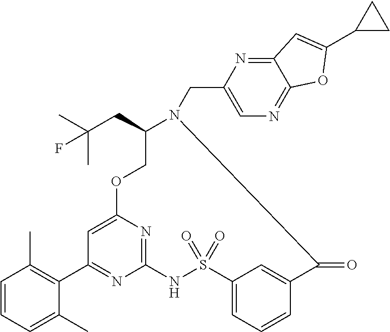 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)F)CO2] | 1.87 | 670.237 | 671.4 | A |
| I-54 | 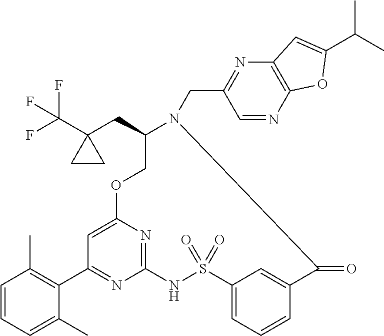 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C(F)(F)F)CC1)CO2] | 1.99 | 720.234 | 721.8 | A |
| I-55 | 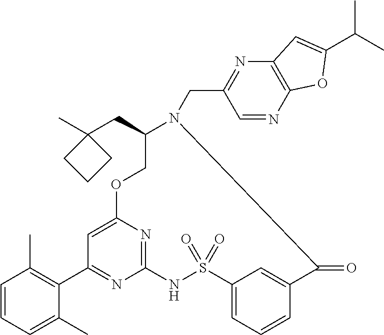 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C)CCC1)CO2] | 2.15 | 680.278 | 681.9 | A |
| I-56 | 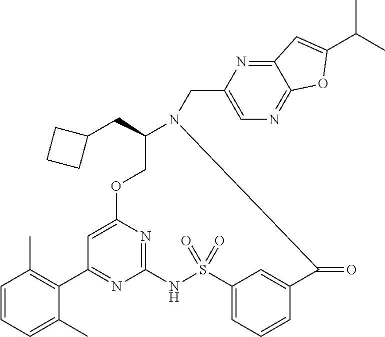 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCC1)CO2] | 2.09 | 666.262 | 667.7 | A |
| I-57 | 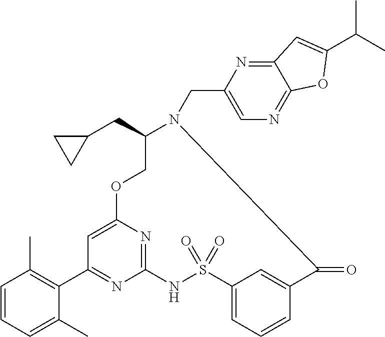 [SMILES: Cc1cccc(C)c1-c1cc2nc(n1)NS(=O)(=O)c1cccc(c1)C(=O)N(Cc1cnc3oc(C(C)C)cc3n1)[C@H](CC1CC1)CO2] | 2.08 | 652.247 | 653.5 | A |
| I-58 | 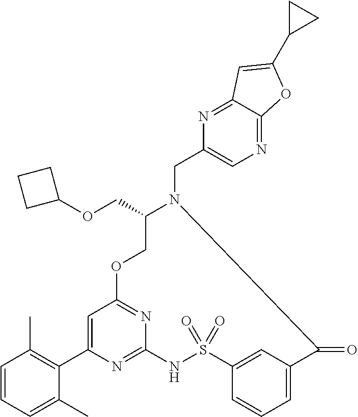 [SMILES: Cc1cccc(C)c1-c1cc2nc(n1)NS(=O)(=O)c1cccc(c1)C(=O)N(Cc1cnc3oc(C4CC4)cc3n1)[C@@H](COC1CCC1)CO2] | 1.63 | 680.242 | 681.5 | A |
| I-59 | 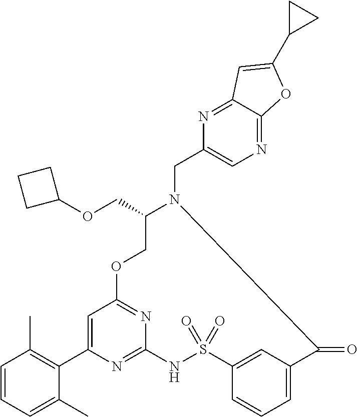 [SMILES: Cc1cccc(C)c1-c1cc2nc(n1)NS(=O)(=O)c1cccc(c1)C(=O)N(Cc1cnc3oc(C4CC4)cc3n1)[C@@H](COC1CCC1)CO2] | 1.63 | 680.242 | 681.4 | A |
| I-60 | 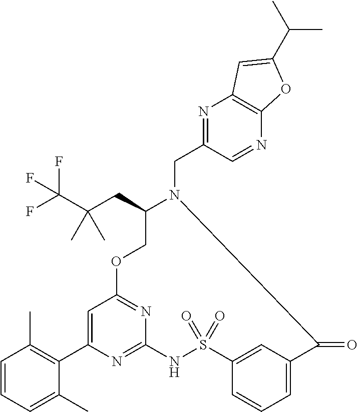 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C(F)(F)F)CO2] | 1.74 | 722.25 | 723.5 | A |
| I-61 | 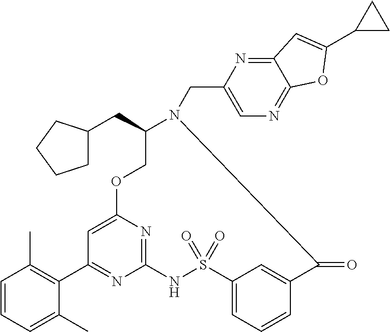 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCC1)CO2] | 2.15 | 678.262 | 679.2 | A |
| I-63 | 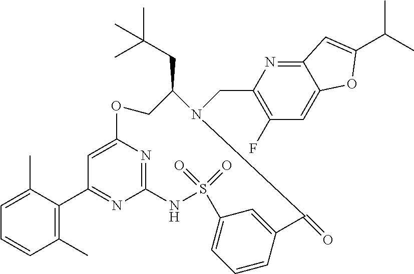 [SMILES: Cc1cccc(C)c1-c1cc2nc(n1)NS(=O)(=O)c1cccc(c1)C(=O)N(Cc1nc3cc(C(C)C)oc3cc1F)[C@H](CC(C)(C)C)CO2] | 5.06 | 685.273 | 686.4 | J |
| I-64 | 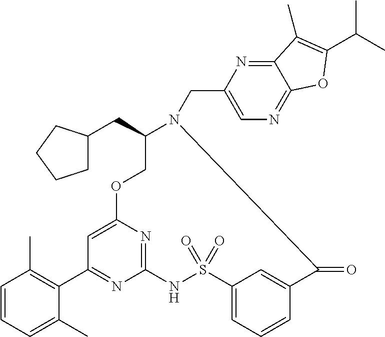 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCC1)CO2] | 2.36 | 694.294 | 695.2 | A |
| I-65 | 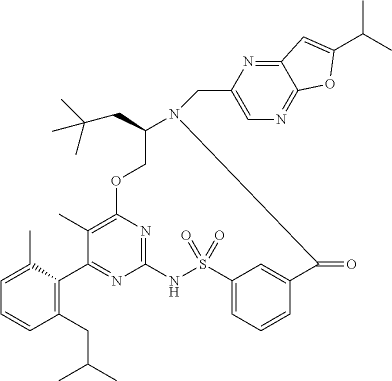 | 2.48 | 724.341 | 725.2 | A |
| I-68 | 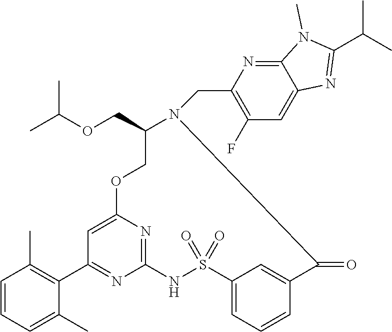 [SMILES: Cc1cccc(C)c1-c1cc2nc(n1)NS(=O)(=O)c1cccc(c1)C(=O)N(Cc1nc3c(cc1F)nc(C(C)C)n3C)[C@H](COC(C)C)CO2] | 1.51 | 701.28 | 702.9 | A |
| I-69 | 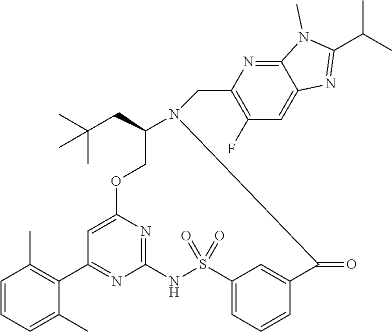 [SMILES: Cc1cccc(C)c1-c1cc2nc(n1)NS(=O)(=O)c1cccc(c1)C(=O)N(Cc1nc3c(cc1F)nc(C(C)C)n3C)[C@H](CC(C)(C)C)CO2] | 1.64 | 699.3 | 700.6 | A |
| I-70 | 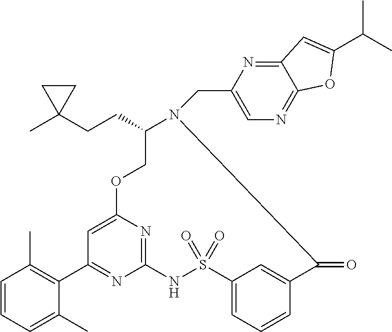 [SMILES: Cc1cccc(C)c1-c1cc2nc(n1)NS(=O)(=O)c1cccc(c1)C(=O)N(Cc1cnc3oc(C(C)C)cc3n1)[C@@H](CCC1(C)CC1)CO2] | 2.24 | 680.278 | 681.4 | A |
| I-72 | 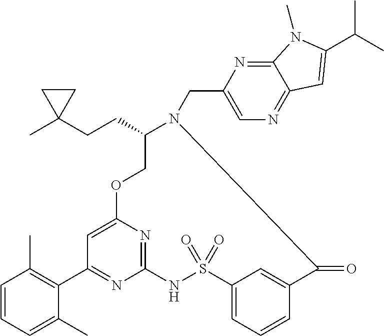 [SMILES: Cc1cccc(C)c1-c1cc2nc(n1)NS(=O)(=O)c1cccc(c1)C(=O)N(Cc1cnc3cc(C(C)C)n(C)c3n1)[C@@H](CCC1(C)CC1)CO2] | 1.94 | 693.31 | 694.4 | A |
| I-73 | 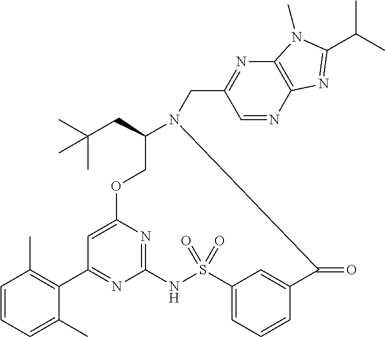 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CO2] | 1.7 | 682.305 | 683.9 | A |
| I-74 | 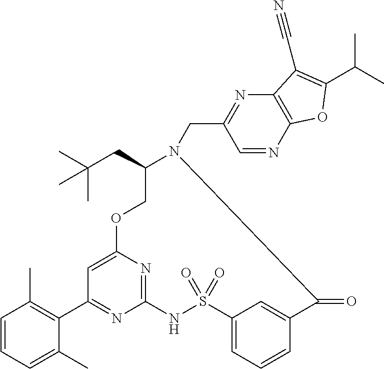 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC(C)(C)C)CO2] | 2.06 | 693.273 | 694.8 | A |
| I-75 | 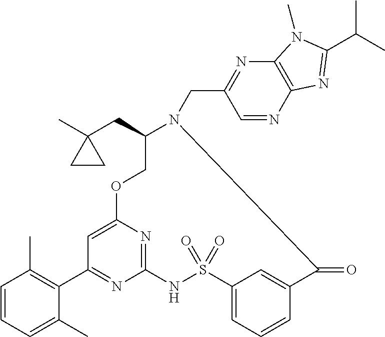 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC1(C)CC1)CO2] | 1.64 | 680.289 | 681.8 | A |
| I-76 | 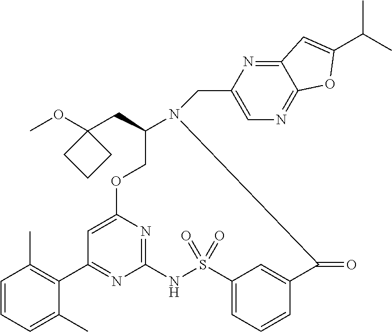 [SMILES: COC1(C[C@@H]2COc3cc(-c4c(C)cccc4C)nc(n3)NS(=O)(=O)c3cccc(c3)C(=O)N2Cc2cnc3oc(C(C)C)cc3n2)CCC1] | 2.05 | 696.273 | 697.2 | A |
| I-77 | 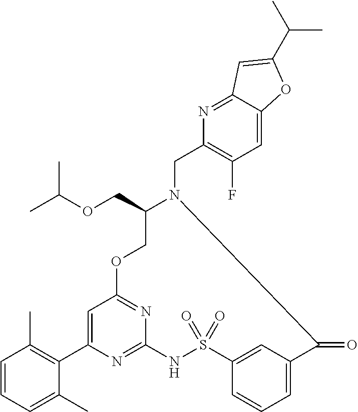 [SMILES: Cc1cccc(C)c1-c1cc2nc(n1)NS(=O)(=O)c1cccc(c1)C(=O)N(Cc1nc3cc(C(C)C)oc3cc1F)[C@H](COC(C)C)CO2] | 1.81 | 687.253 | 688.5 | A |
| I-78 | 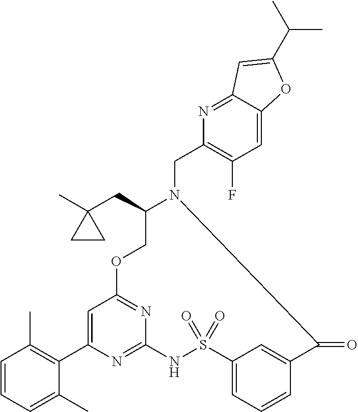 [SMILES: Cc1cccc(C)c1-c1cc2nc(n1)NS(=O)(=O)c1cccc(c1)C(=O)N(Cc1nc3cc(C(C)C)oc3cc1F)[C@H](CC1(C)CC1)CO2] | 1.85 | 683.258 | 684.5 | A |
| I-79 | 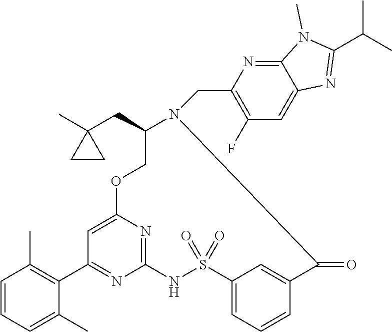 [SMILES: Cc1cccc(C)c1-c1cc2nc(n1)NS(=O)(=O)c1cccc(c1)C(=O)N(Cc1nc3c(cc1F)nc(C(C)C)n3C)[C@H](CC1(C)CC1)CO2] | 1.59 | 697.285 | 698.5 | A |
| I-80 | 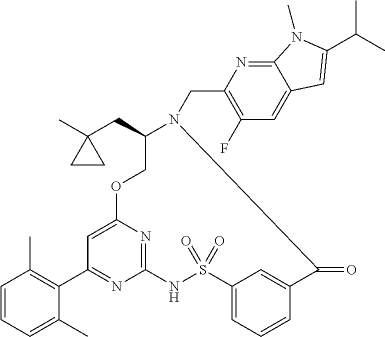 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC1(C)CC1)CO2] | 2.39 | 696.289 | 697.4 | A |
| I-81 | 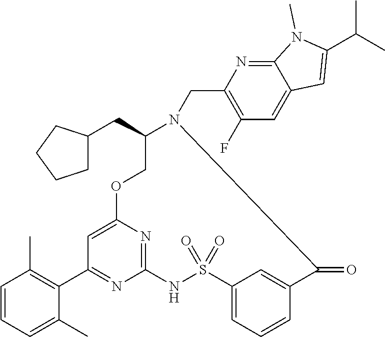 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC1CCCC1)CO2] | 2.51 | 710.305 | 711.4 | A |
| I-82 | 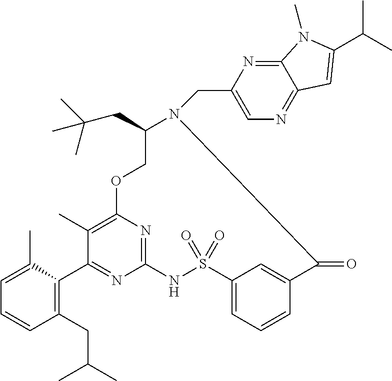 | 2.17 | 737.372 | 738.2 | A |
| I-83 | 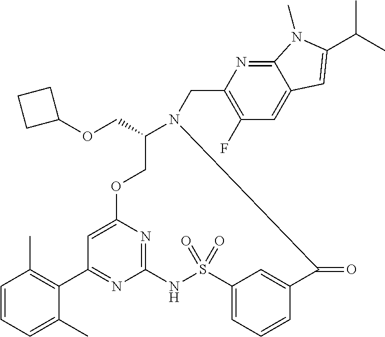 [SMILES: Cc1cccc(C)c1-c1cc2nc(n1)NS(=O)(=O)c1cccc(c1)C(=O)N(Cc1nc3c(cc1F)cc(C(C)C)n3C)[C@@H](COC1CCC1)CO2] | 2.36 | 712.284 | 713.6 | A |
| I-84 | 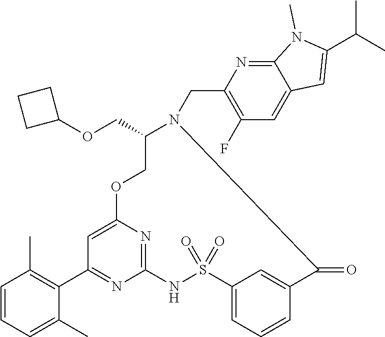 [SMILES: Cc1cccc(C)c1-c1cc2nc(n1)NS(=O)(=O)c1cccc(c1)C(=O)N(Cc1nc3c(cc1F)cc(C(C)C)n3C)[C@@H](COC1CCC1)CO2] | 2.4 | 712.284 | 713.4 | A |
| I-85 | 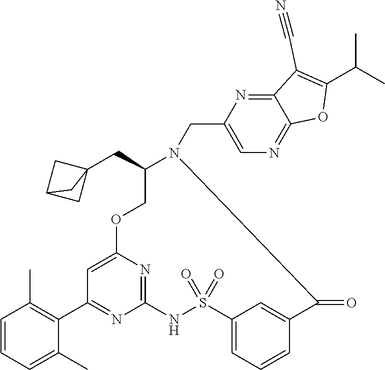 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC13CC(C1)C3)CO2] | 2.1 | 703.258 | 704.2 | A |
| I-86 | 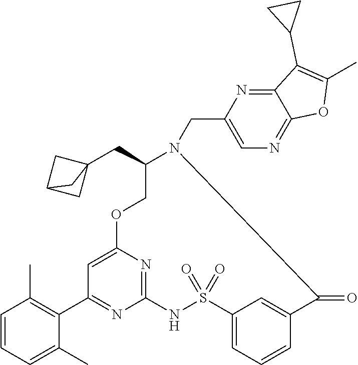 [SMILES: Cc1cccc(C)c1-c1cc2nc(n1)NS(=O)(=O)c1cccc(c1)C(=O)N(Cc1cnc3oc(C)c(C4CC4)c3n1)[C@H](CC13CC(C1)C3)CO2] | 2.21 | 690.262 | 691.2 | A |
| I-89 | 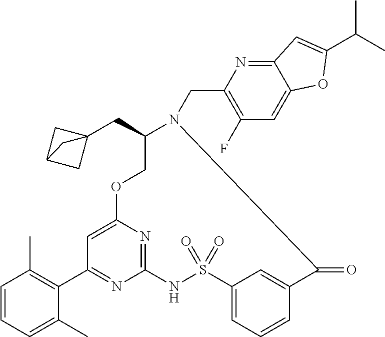 [SMILES: Cc1cccc(C)c1-c1cc2nc(n1)NS(=O)(=O)c1cccc(c1)C(=O)N(Cc1nc3cc(C(C)C)oc3cc1F)[C@H](CC13CC(C1)C3)CO2] | 2.34 | 695.258 | 696.4 | A |
| I-90 | 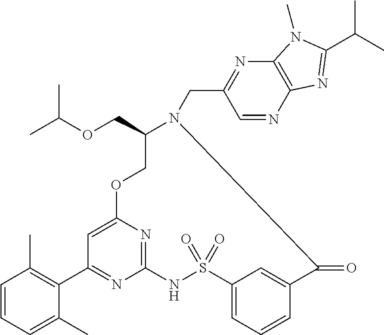 [SMILES: Cc1cccc(C)c1-c1cc2nc(n1)NS(=O)(=O)c1cccc(c1)C(=O)N(Cc1cnc3nc(C(C)C)n(C)c3n1)[C@H](COC(C)C)CO2] | 1.58 | 684.284 | 685.9 | A |
| I-91 | 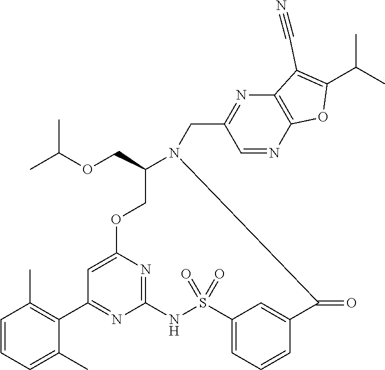 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](COC(C)C)CO2] | 1.95 | 695.253 | 696.6 | A |
| I-92 | 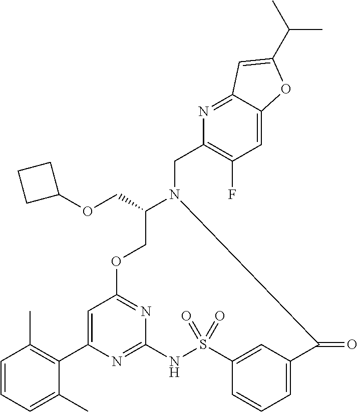 [SMILES: Cc1cccc(C)c1-c1cc2nc(n1)NS(=O)(=O)c1cccc(c1)C(=O)N(Cc1nc3cc(C(C)C)oc3cc1F)[C@@H](COC1CCC1)CO2] | 1.85 | 699.253 | 700.6 | A |
| I-93 | 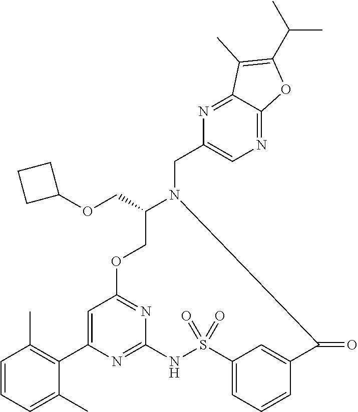 [SMILES: Cc1cccc(C)c1-c1cc2nc(n1)NS(=O)(=O)c1cccc(c1)C(=O)N(Cc1cnc3oc(C(C)C)c(C)c3n1)[C@@H](COC1CCC1)CO2] | 1.78 | 696.273 | 697.5 | A |
| I-94 | 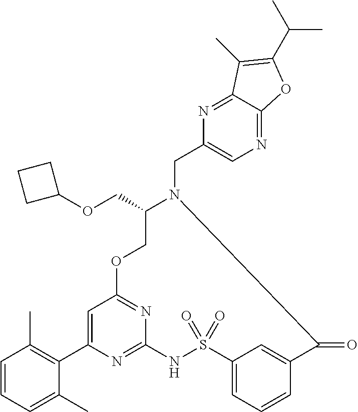 [SMILES: Cc1cccc(C)c1-c1cc2nc(n1)NS(=O)(=O)c1cccc(c1)C(=O)N(Cc1cnc3oc(C(C)C)c(C)c3n1)[C@@H](COC1CCC1)CO2] | 1.78 | 696.273 | 697.6 | A |
| I-95 | 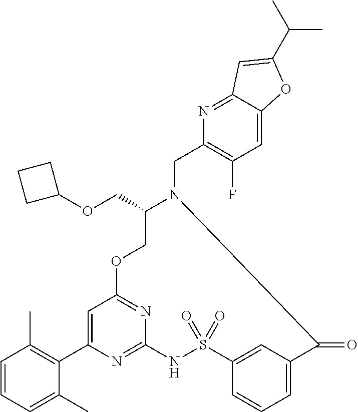 [SMILES: Cc1cccc(C)c1-c1cc2nc(n1)NS(=O)(=O)c1cccc(c1)C(=O)N(Cc1nc3cc(C(C)C)oc3cc1F)[C@@H](COC1CCC1)CO2] | 1.85 | 699.253 | 700.5 | A |
| I-97 | 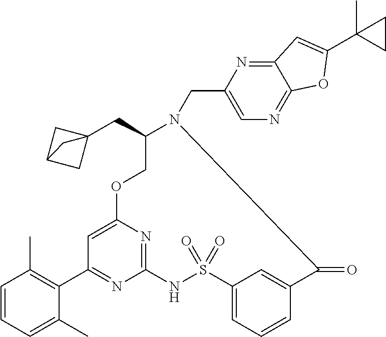 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC13CC(C1)C3)CO2] | 2.26 | 690.262 | 691.3 | A |
| I-100 | 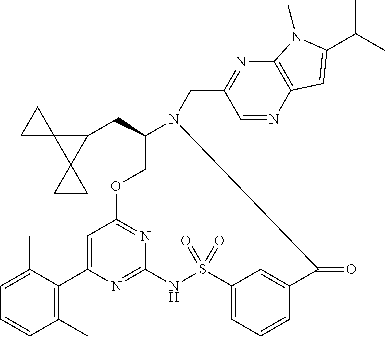 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1C3(CC3)C13CC3)CO2] | 1.91 | 717.31 | 718.9 | A |
| I-101 | 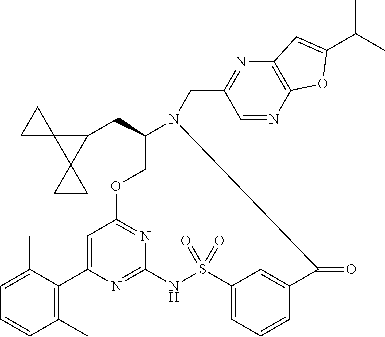 [SMILES: Cc1cccc(C)c1-c1cc2nc(n1)NS(=O)(=O)c1cccc(c1)C(=O)N(Cc1cnc3oc(C(C)C)cc3n1)[C@H](CC1C3(CC3)C13CC3)CO2] | 2.19 | 704.278 | 705.9 | A |
| I-102 | 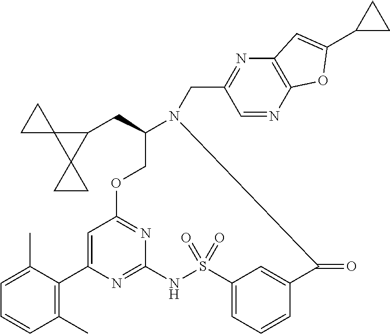 [SMILES: Cc1cccc(C)c1-c1cc2nc(n1)NS(=O)(=O)c1cccc(c1)C(=O)N(Cc1cnc3oc(C4CC4)cc3n1)[C@H](CC1C3(CC3)C13CC3)CO2] | 2.11 | 702.262 | 703.8 | A |
| I-103 | 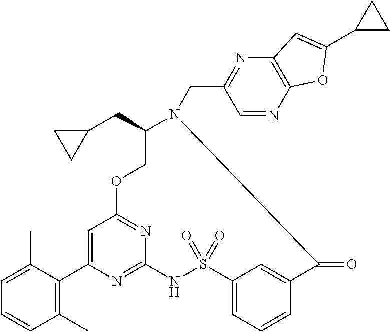 [SMILES: Cc1cccc(C)c1-c1cc2nc(n1)NS(=O)(=O)c1cccc(c1)C(=O)N(Cc1cnc3oc(C4CC4)cc3n1)[C@H](CC1CC1)CO2] | 1.88 | 650.231 | 651.8 | A |
| I-105 | 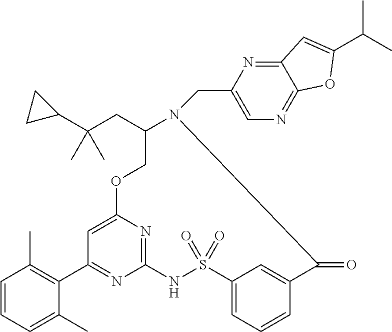 [SMILES: Cc1cccc(C)c1-c1cc2nc(n1)NS(=O)(=O)c1cccc(c1)C(=O)N(Cc1cnc3oc(C(C)C)cc3n1)C(CC(C)(C)C1CC1)CO2] | 2.26 | 694.294 | 695.7 | A |
| I-106 | 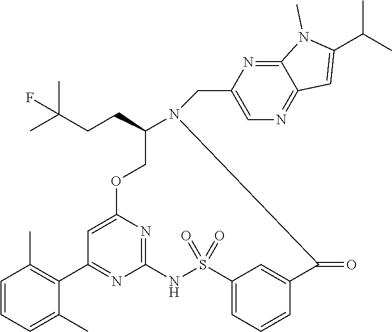 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)F)CO2] | 1.75 | 699.3 | 700.7 | A |
| I-107 | 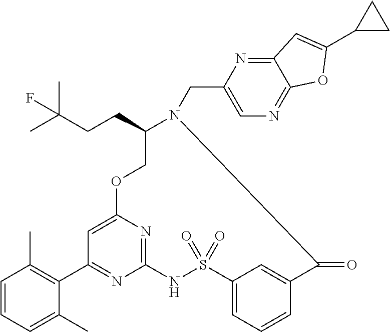 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)F)CO2] | 1.91 | 684.253 | 685.9 | A |
| I-108 | 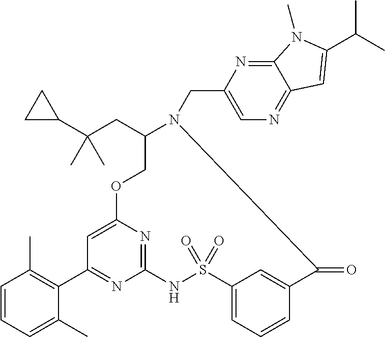 [SMILES: Cc1cccc(C)c1-c1cc2nc(n1)NS(=O)(=O)c1cccc(c1)C(=O)N(Cc1cnc3cc(C(C)C)n(C)c3n1)C(CC(C)(C)C1CC1)CO2] | 1.91 | 707.325 | 708.5 | A |
| I-109 | 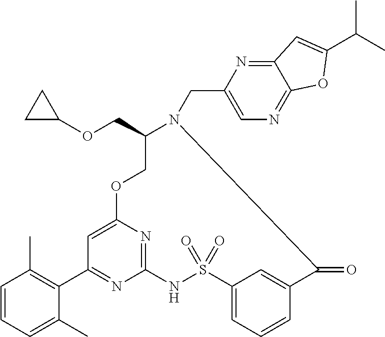 [SMILES: Cc1cccc(C)c1-c1cc2nc(n1)NS(=O)(=O)c1cccc(c1)C(=O)N(Cc1cnc3oc(C(C)C)cc3n1)[C@H](COC1CC1)CO2] | 2.02 | 668.242 | 669.4 | A |
| I-110 | 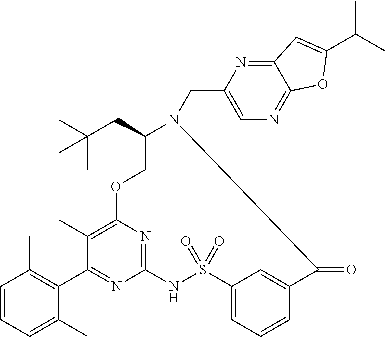 [SMILES: Cc1cccc(C)c1-c1nc2nc(c1C)OC[C@@H](CC(C)(C)C)N(Cc1cnc3oc(C(C)C)cc3n1)C(=O)c1cccc(c1)S(=O)(=O)N2] | 2.27 | 682.294 | 683.4 | A |
| I-111 | 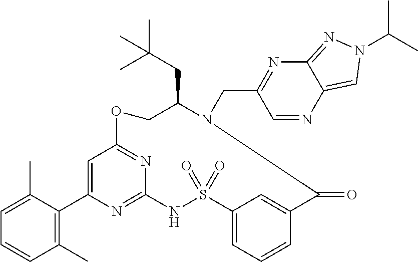 [SMILES: Cc1cccc(C)c1-c1cc2nc(n1)NS(=O)(=O)c1cccc(c1)C(=O)N(Cc1cnc3cn(C(C)C)nc3n1)[C@H](CC(C)(C)C)CO2] | 4.17 | 668.289 | 669.1 | J |
| I-112 | 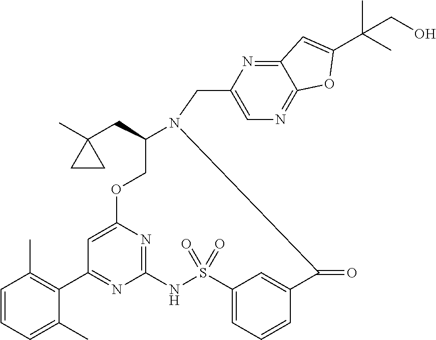 [SMILES: Cc1cccc(C)c1-c1cc2nc(n1)NS(=O)(=O)c1cccc(c1)C(=O)N(Cc1cnc3oc(C(C)(C)CO)cc3n1)[C@H](CC1(C)CC1)CO2] | 1.88 | 696.273 | 697.4 | A |
| I-114 | 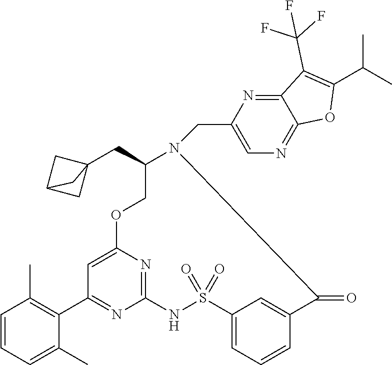 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@H](CC13CC(C1)C3)CO2] | 2.26 | 746.25 | 747.9 | A |
| I-115 | 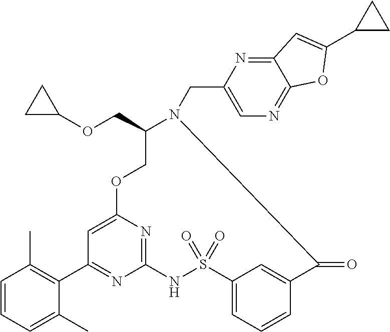 [SMILES: Cc1cccc(C)c1-c1cc2nc(n1)NS(=O)(=O)c1cccc(c1)C(=O)N(Cc1cnc3oc(C4CC4)cc3n1)[C@H](COC1CC1)CO2] | 1.93 | 666.226 | 667.4 | A |
| I-116 | 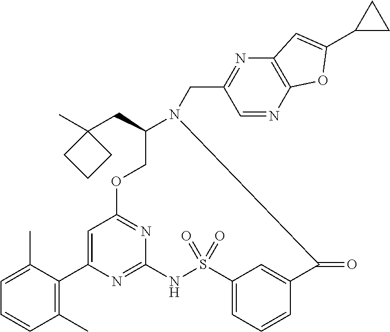 [SMILES: Cc1cccc(C)c1-c1cc2nc(n1)NS(=O)(=O)c1cccc(c1)C(=O)N(Cc1cnc3oc(C4CC4)cc3n1)[C@H](CC1(C)CCC1)CO2] | 2.07 | 678.262 | 679.7 | A |
| I-118 | 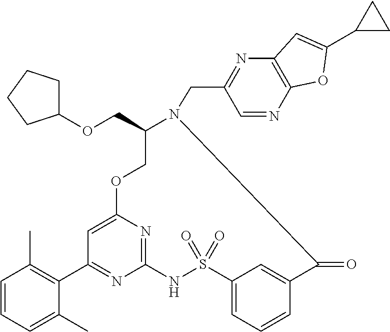 [SMILES: Cc1cccc(C)c1-c1cc2nc(n1)NS(=O)(=O)c1cccc(c1)C(=O)N(Cc1cnc3oc(C4CC4)cc3n1)[C@H](COC1CCCC1)CO2] | 2.07 | 694.257 | 695.7 | A |
| I-119 | 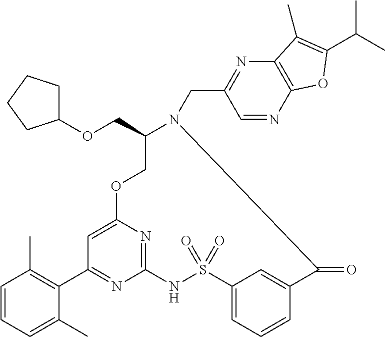 [SMILES: Cc1cccc(C)c1-c1cc2nc(n1)NS(=O)(=O)c1cccc(c1)C(=O)N(Cc1cnc3oc(C(C)C)c(C)c3n1)[C@H](COC1CCCC1)CO2] | 2.23 | 710.289 | 711.9 | A |
| I-120 | 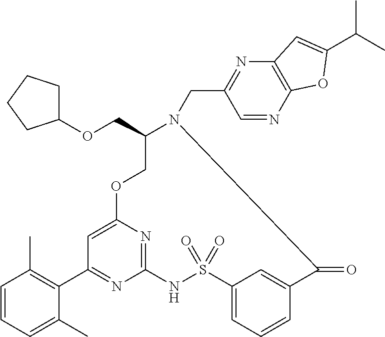 [SMILES: Cc1cccc(C)c1-c1cc2nc(n1)NS(=O)(=O)c1cccc(c1)C(=O)N(Cc1cnc3oc(C(C)C)cc3n1)[C@H](COC1CCCC1)CO2] | 2.14 | 696.273 | 697.7 | A |
| I-122 | 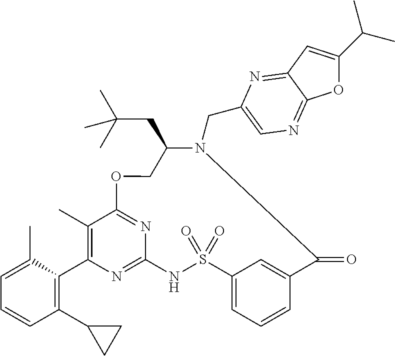 | 2.27 | 708.309 | 709.71 | A |
| I-124 | 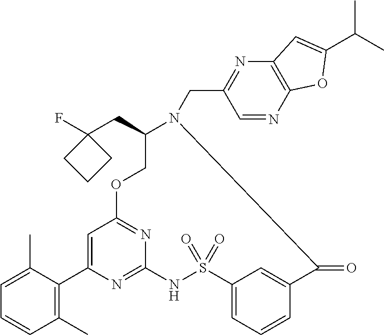 [SMILES: Cc1cccc(C)c1-c1cc2nc(n1)NS(=O)(=O)c1cccc(c1)C(=O)N(Cc1cnc3oc(C(C)C)cc3n1)[C@H](CC1(F)CCC1)CO2] | 198 | 684.253 | 685.8 | A |
| I-125 | 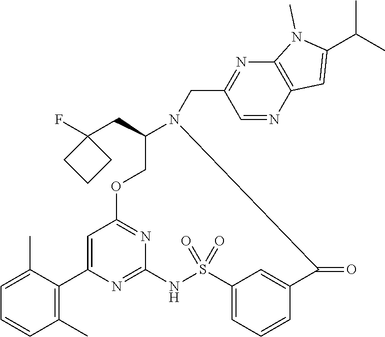 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(F)CCC1)CO2] | 1.73 | 697.285 | 698.8 | A |
| I-127 | 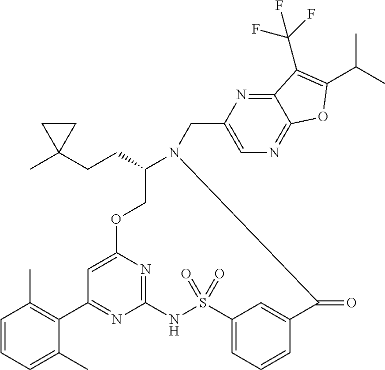 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@@H](CCC1(C)CC1)CO2] | 2.19 | 748.265 | 749.1 | A |
| I-129 | 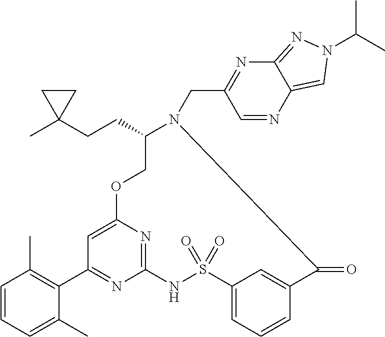 [SMILES: Cc1cccc(C)c1-c1cc2nc(n1)NS(=O)(=O)c1cccc(c1)C(=O)N(Cc1cnc3cn(C(C)C)nc3n1)[C@@H](CCC1(C)CC1)CO2] | 1.78 | 680.289 | 681.2 | A |
| I-130 | 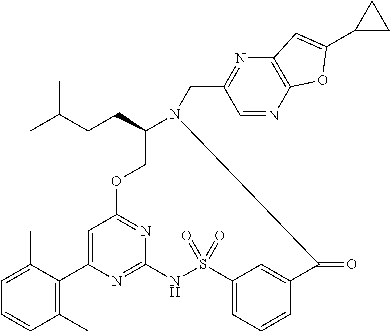 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)C)CO2] | 1.96 | 666.262 | 667.2 | A |
| I-131 | 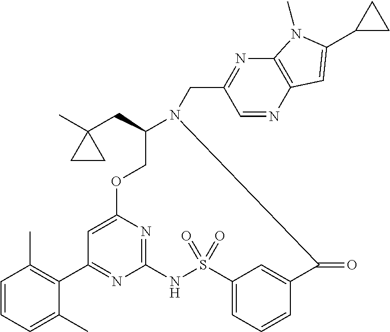 [SMILES: Cc1cccc(C)c1-c1cc2nc(n1)NS(=O)(=O)c1cccc(c1)C(=O)N(Cc1cnc3cc(C4CC4)n(C)c3n1)[C@H](CC1(C)CC1)CO2] | 1.6 | 677.278 | 678.1 | A |
| I-132 | 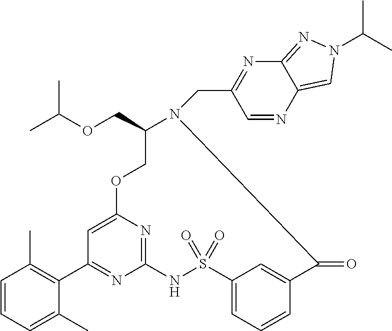 [SMILES: Cc1cccc(C)c1-c1cc2nc(n1)NS(=O)(=O)c1cccc(c1)C(=O)N(Cc1cnc3cn(C(C)C)nc3n1)[C@H](COC(C)C)CO2] | 1.62 | 670.269 | 671.1 | A |
| I-133 | 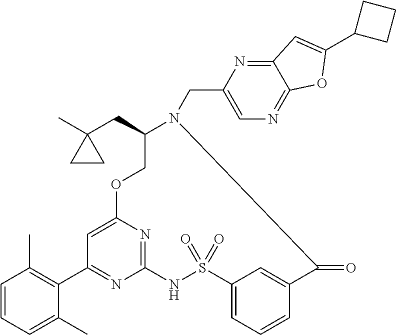 [SMILES: Cc1cccc(C)c1-c1cc2nc(n1)NS(=O)(=O)c1cccc(c1)C(=O)N(Cc1cnc3oc(C4CCC4)cc3n1)[C@H](CC1(C)CC1)CO2] | 2.17 | 678.262 | 679.4 | A |
| I-135 | 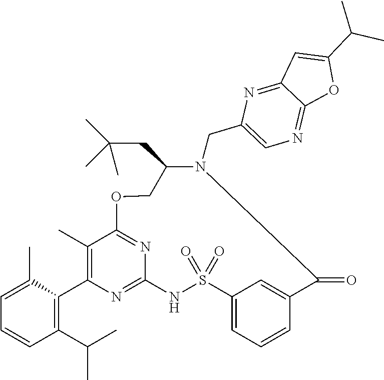 | 2.28 | 710.325 | 711.76 | A |
| I-136 | 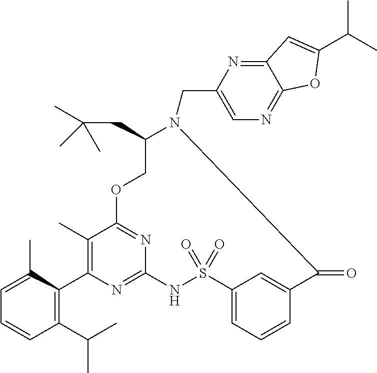 | 2.24 | 710.325 | 711.7 | A |
| I-137 | 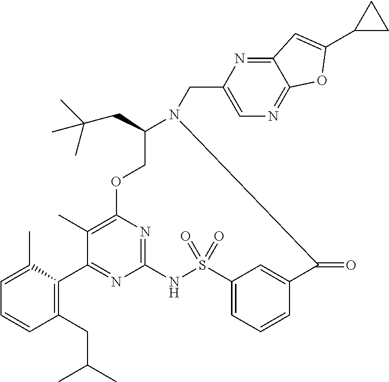 | 2.27 | 722.325 | 723.97 | A |
| I-138 | 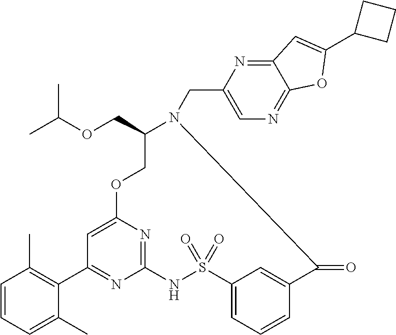 [SMILES: Cc1cccc(C)c1-c1cc2nc(n1)NS(=O)(=O)c1cccc(c1)C(=O)N(Cc1cnc3oc(C4CCC4)cc3n1)[C@H](COC(C)C)CO2] | 2.11 | 682.257 | 683.4 | A |
| I-139 | 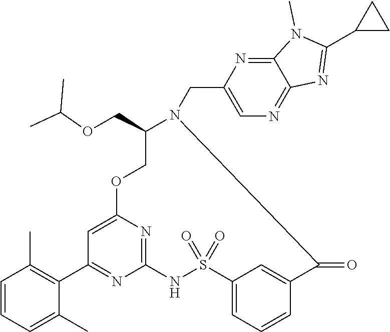 [SMILES: Cc1cccc(C)c1-c1cc2nc(n1)NS(=O)(=O)c1cccc(c1)C(=O)N(Cc1cnc3nc(C4CC4)n(C)c3n1)[C@H](COC(C)C)CO2] | 1.53 | 682.269 | 683.1 | A |
| I-140 | 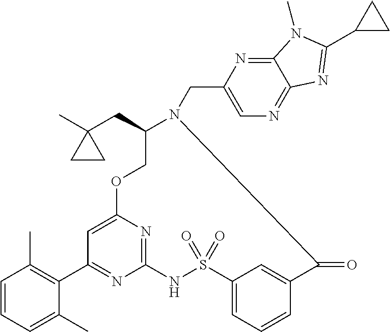 [SMILES: Cc1cccc(C)c1-c1cc2nc(n1)NS(=O)(=O)c1cccc(c1)C(=O)N(Cc1cnc3nc(C4CC4)n(C)c3n1)[C@H](CC1(C)CC1)CO2] | 1.58 | 678.274 | 679.2 | A |
| I-141 | 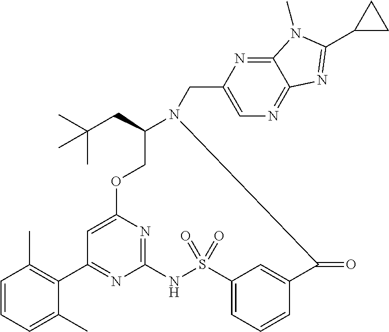 [SMILES: Cc1cccc(C)c1-c1cc2nc(n1)NS(=O)(=O)c1cccc(c1)C(=O)N(Cc1cnc3nc(C4CC4)n(C)c3n1)[C@H](CC(C)(C)C)CO2] | 1.63 | 680.289 | 681.3 | A |
| I-142 | 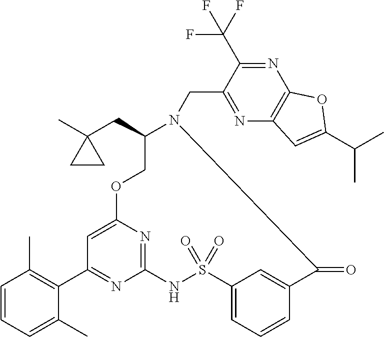 [SMILES: Cc1cccc(C)c1-c1cc2nc(n1)NS(=O)(=O)c1cccc(c1)C(=O)N(Cc1nc3cc(C(C)C)oc3nc1C(F)(F)F)[C@H](CC1(C)CC1)CO2] | 2.16 | 734.25 | 735.1 | A |
| I-144 | 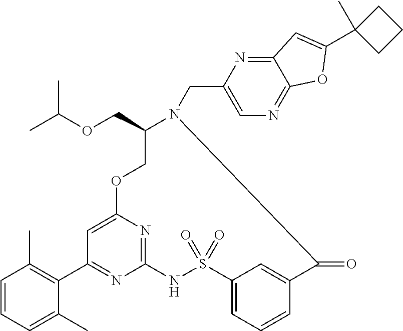 [SMILES: Cc1cccc(C)c1-c1cc2nc(n1)NS(=O)(=O)c1cccc(c1)C(=O)N(Cc1cnc3oc(C4(C)CCC4)cc3n1)[C@H](COC(C)C)CO2] | 2.22 | 696.273 | 697.4 | A |
| I-145 | 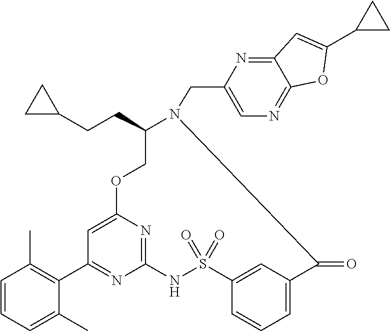 [SMILES: Cc1cccc(C)c1-c1cc2nc(n1)NS(=O)(=O)c1cccc(c1)C(=O)N(Cc1cnc3oc(C4CC4)cc3n1)[C@H](CCC1CC1)CO2] | 1.95 | 664.247 | 665.7 | A |
| I-146 | 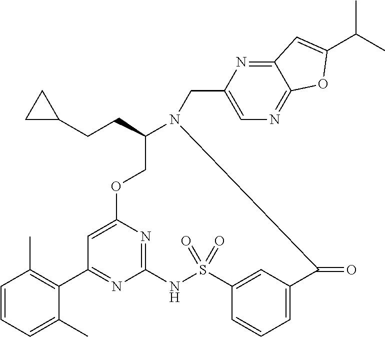 [SMILES: Cc1cccc(C)c1-c1cc2nc(n1)NS(=O)(=O)c1cccc(c1)C(=O)N(Cc1cnc3oc(C(C)C)cc3n1)[C@H](CCC1CC1)CO2] | 2.03 | 666.262 | 667.7 | A |
| I-150 | 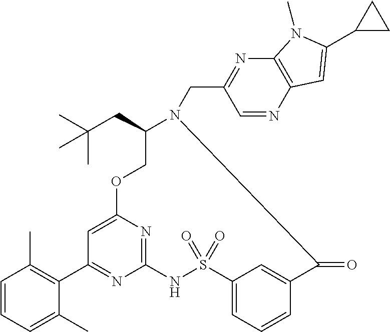 [SMILES: Cc1cccc(C)c1-c1cc2nc(n1)NS(=O)(=O)c1cccc(c1)C(=O)N(Cc1cnc3cc(C4CC4)n(C)c3n1)[C@H](CC(C)(C)C)CO2] | 1.63 | 679.294 | 680.2 | A |
| I-151 | 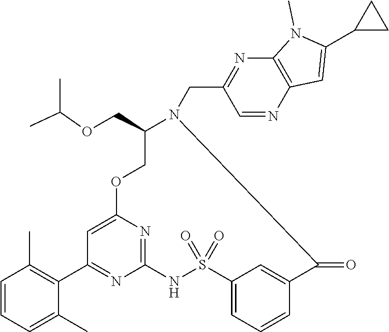 [SMILES: Cc1cccc(C)c1-c1cc2nc(n1)NS(=O)(=O)c1cccc(c1)C(=O)N(Cc1cnc3cc(C4CC4)n(C)c3n1)[C@H](COC(C)C)CO2] | 1.56 | 681.273 | 682.1 | A |
| I-152 | 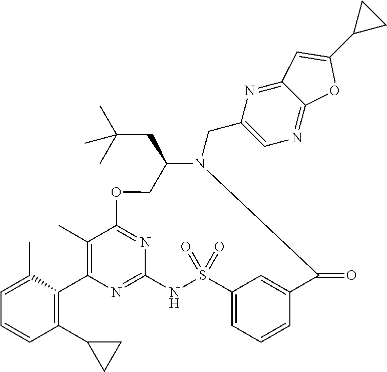 | 2.13 | 706.294 | 707.96 | A |
| I-153 | 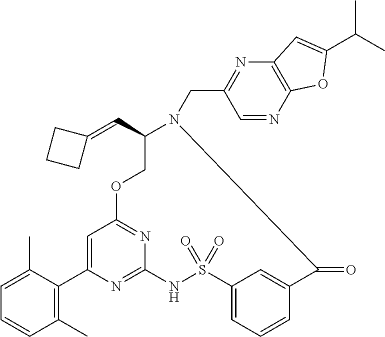 [SMILES: Cc1cccc(C)c1-c1cc2nc(n1)NS(=O)(=O)c1cccc(c1)C(=O)N(Cc1cnc3oc(C(C)C)cc3n1)[C@H](C=C1CCC1)CO2] | 2.05 | 664.247 | 665.6 | A |
| I-155 | 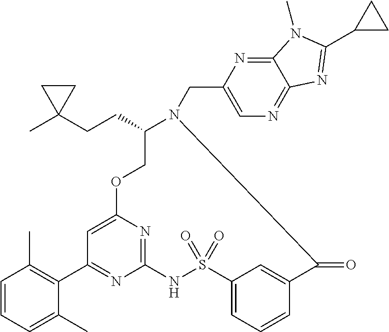 [SMILES: Cc1cccc(C)c1-c1cc2nc(n1)NS(=O)(=O)c1cccc(c1)C(=O)N(Cc1cnc3nc(C4CC4)n(C)c3n1)[C@@H](CCC1(C)CC1)CO2] | 1.7 | 692.289 | 693.7 | A |
| I-156 | 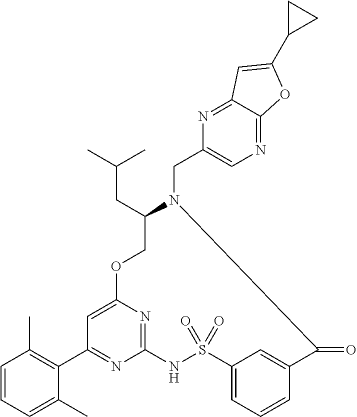 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)C)CO2] | 1.93 | 652.247 | 653.8 | A |
| I-158 | 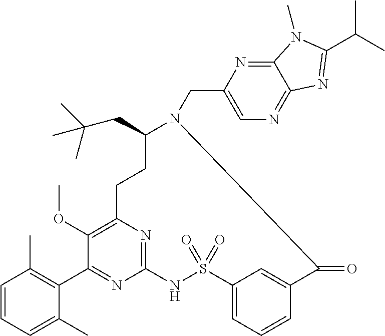 [SMILES: COc1c2nc(nc1-c1c(C)cccc1C)NS(=O)(=O)c1cccc(c1)C(=O)N(Cc1cnc3nc(C(C)C)n(C)c3n1)[C@H](CC(C)(C)C)CC2] | 2.02 | 709.341 | 710.5 | A |
| I-162 | 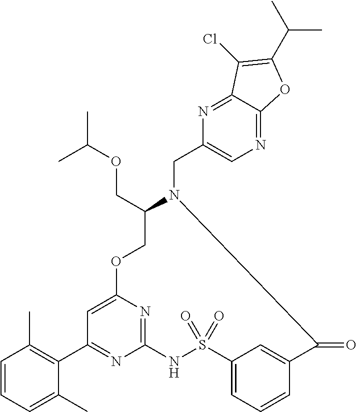 [SMILES: Cc1cccc(C)c1-c1cc2nc(n1)NS(=O)(=O)c1cccc(c1)C(=O)N(Cc1cnc3oc(C(C)C)c(Cl)c3n1)[C@H](COC(C)C)CO2] | 2.09 | 704.218 | 705.6 | A |
| I-163 | 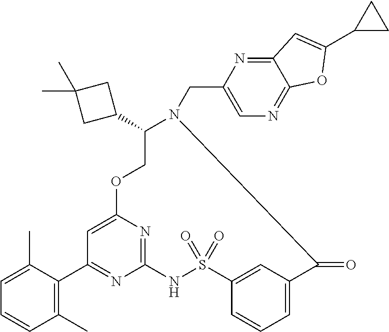 [SMILES: Cc1cccc(C)c1-c1cc2nc(n1)NS(=O)(=O)c1cccc(c1)C(=O)N(Cc1cnc3oc(C4CC4)cc3n1)[C@@H](C1CC(C)(C)C1)CO2] | 2.05 | 678.262 | 679.8 | A |
| I-164 | 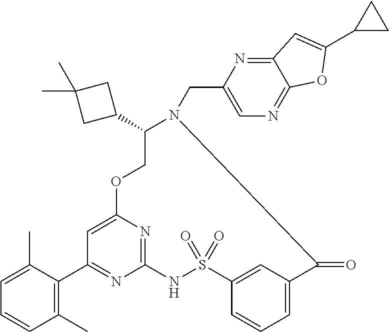 [SMILES: Cc1cccc(C)c1-c1cc2nc(n1)NS(=O)(=O)c1cccc(c1)C(=O)N(Cc1cnc3oc(C4CC4)cc3n1)[C@@H](C1CC(C)(C)C1)CO2] | 2.05 | 678.262 | 679.7 | A |
| I-167 | 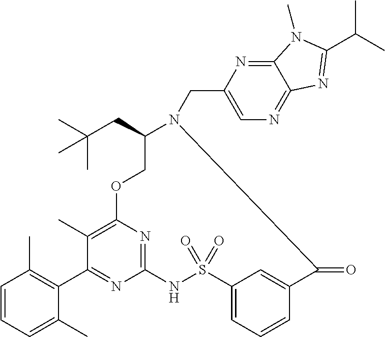 [SMILES: Cc1cccc(C)c1-c1nc2nc(c1C)OC[C@@H](CC(C)(C)C)N(Cc1cnc3nc(C(C)C)n(C)c3n1)C(=O)c1cccc(c1)S(=O)(=O)N2] | 1.81 | 696.321 | 697.7 | A |
| I-168 | 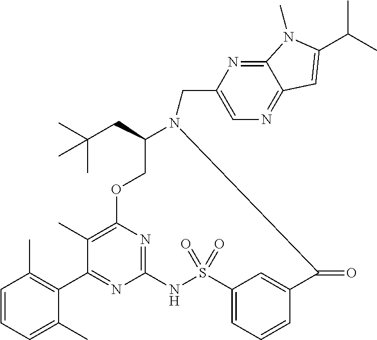 [SMILES: Cc1cccc(C)c1-c1nc2nc(c1C)OC[C@@H](CC(C)(C)C)N(Cc1cnc3cc(C(C)C)n(C)c3n1)C(=O)c1cccc(c1)S(=O)(=O)N2] | 1.94 | 695.325 | 696.7 | A |
| I-170 | 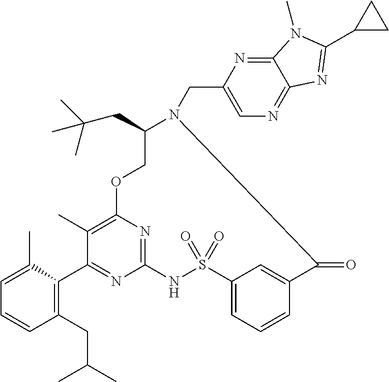 | 1.88 | 736.352 | 737.2 | A |
| I-171 | 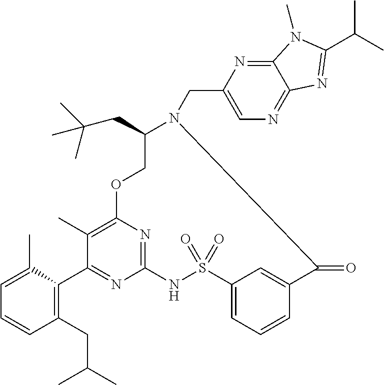 | 2.9 | 738.368 | 739.6 | A (5 min gradient) |
| I-172 | 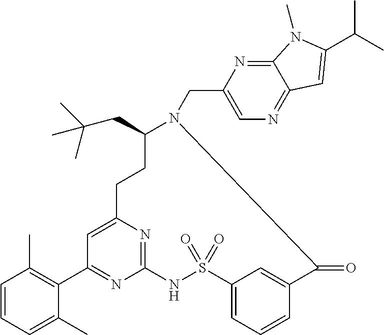 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C)CC2] | 1.93 | 679.33 | 680.5 | A |
| I-173 | 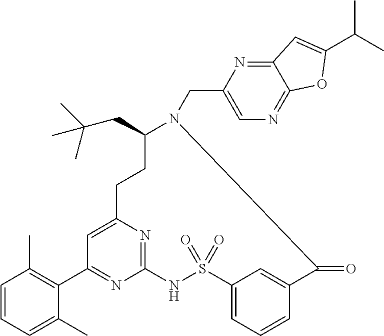 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C)CC2] | 2.26 | 666.299 | 667.4 | A |
| I-174 | 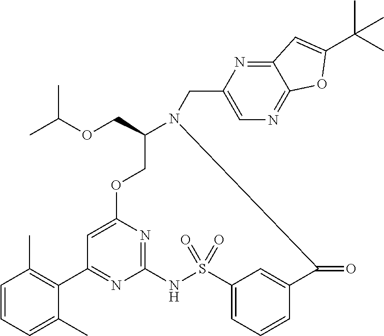 [SMILES: Cc1cccc(C)c1-c1cc2nc(n1)NS(=O)(=O)c1cccc(c1)C(=O)N(Cc1cnc3oc(C(C)(C)C)cc3n1)[C@H](COC(C)C)CO2] | 2.16 | 684.273 | 685.3 | A |
| I-178 | 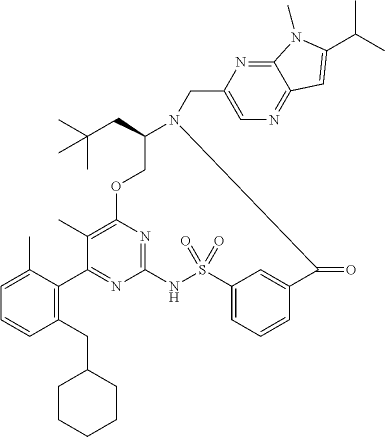 [SMILES: Cc1cccc(CC2CCCCC2)c1-c1nc2nc(c1C)OC[C@@H](CC(C)(C)C)N(Cc1cnc3cc(C(C)C)n(C)c3n1)C(=O)c1cccc(c1)S(=O)(=O)N2] | 2.23 | 777.404 | 778.94 | A |
| I-179 | 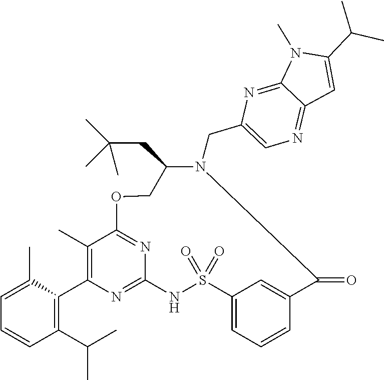 | 2.08 | 723.357 | 724.6 | A |
| I-180 | 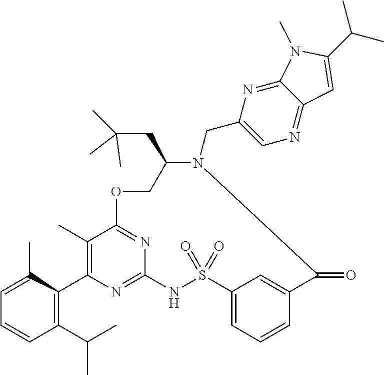 | 1.9 | 723.357 | 724.2 | A |
| I-181 | 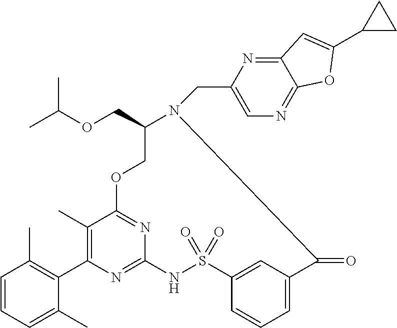 [SMILES: Cc1cccc(C)c1-c1nc2nc(c1C)OC[C@@H](COC(C)C)N(Cc1cnc3oc(C4CC4)cc3n1)C(=O)c1cccc(c1)S(=O)(=O)N2] | 1.89 | 682.257 | 683.9 | A |
| I-182 | 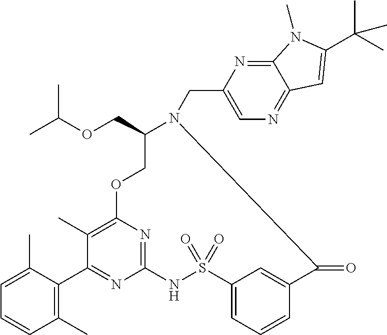 [SMILES: Cc1cccc(C)c1-c1nc2nc(c1C)OC[C@@H](COC(C)C)N(Cc1cnc3cc(C(C)(C)C)n(C)c3n1)C(=O)c1cccc(c1)S(=O)(=O)N2] | 1.78 | 711.32 | 712.1 | A |
| I-183 | 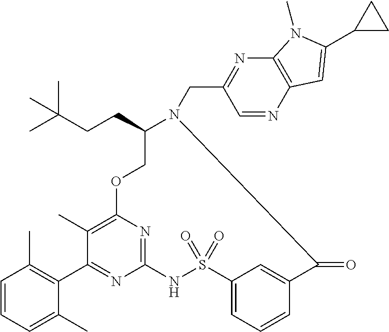 [SMILES: Cc1cccc(C)c1-c1nc2nc(c1C)OC[C@@H](CCC(C)(C)C)N(Cc1cnc3cc(C4CC4)n(C)c3n1)C(=O)c1cccc(c1)S(=O)(=O)N2] | 1.8 | 707.325 | 708.2 | A |
| I-184 | 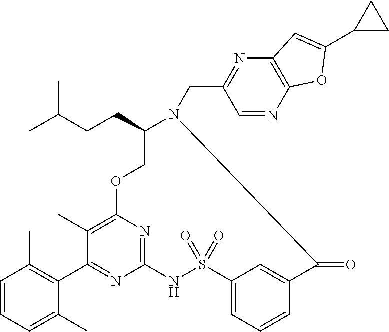 [SMILES: Cc1cccc(C)c1-c1nc2nc(c1C)OC[C@@H](CCC(C)C)N(Cc1cnc3oc(C4CC4)cc3n1)C(=O)c1cccc(c1)S(=O)(=O)N2] | 2.06 | 694.294 | 695.2 | A |
| I-185 | 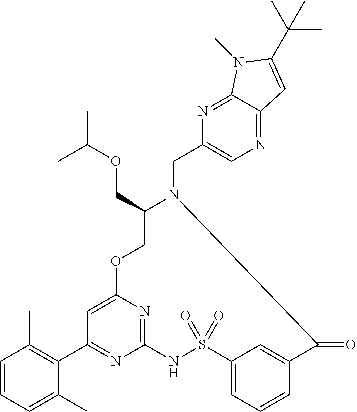 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](COC(C)C)CO2] | 1.76 | 697.305 | 698.6 | A |
| I-186 | 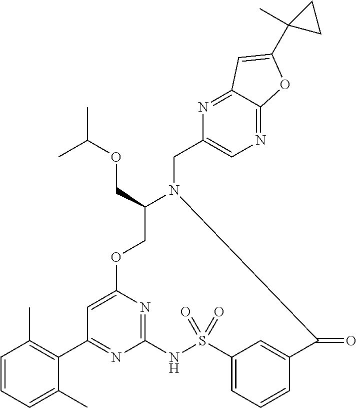 [SMILES: Cc1cccc(C)c1-c1cc2nc(n1)NS(=O)(=O)c1cccc(c1)C(=O)N(Cc1cnc3oc(C4(C)CC4)cc3n1)[C@H](COC(C)C)CO2] | 1.96 | 682.257 | 683.7 | A |
| I-189 | 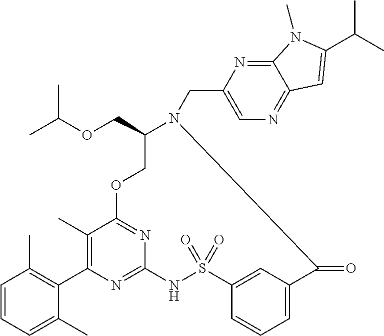 [SMILES: Cc1cccc(C)c1-c1nc2nc(c1C)OC[C@@H](COC(C)C)N(Cc1cnc3cc(C(C)C)n(C)c3n1)C(=O)c1cccc(c1)S(=O)(=O)N2] | 1.71 | 697.305 | 698.2 | A |
| I-190 | 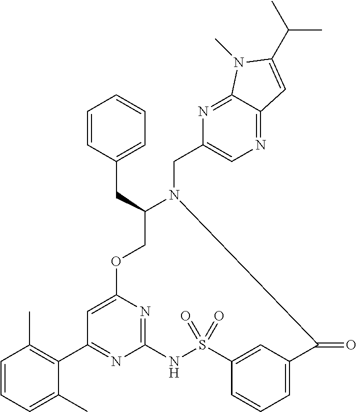 [SMILES: Cc1cccc(C)c1-c1cc2nc(n1)NS(=O)(=O)c1cccc(c1)C(=O)N(Cc1cnc3cc(C(C)C)n(C)c3n1)[C@H](Cc1ccccc1)CO2] | 1.7 | 701.278 | 702.7 | A |
| I-192 | 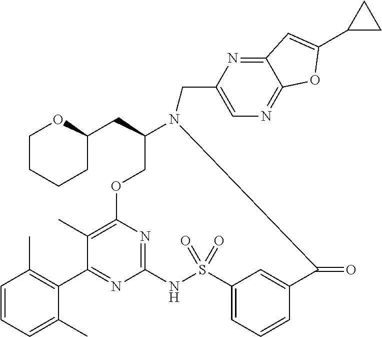 [SMILES: Cc1cccc(C)c1-c1nc2nc(c1C)OC[C@@H](C[C@H]1CCCCO1)N(Cc1cnc3oc(C4CC4)cc3n1)C(=O)c1cccc(c1)S(=O)(=O)N2] | 1.84 | 708.273 | 709.1 | A |
| I-193 | 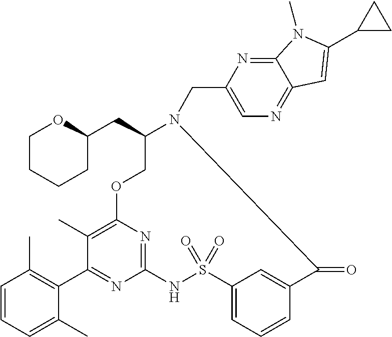 [SMILES: Cc1cccc(C)c1-c1nc2nc(c1C)OC[C@@H](C[C@H]1CCCCO1)N(Cc1cnc3cc(C4CC4)n(C)c3n1)C(=O)c1cccc(c1)S(=O)(=O)N2] | 1.57 | 721.305 | 722.1 | A |
| I-194 | 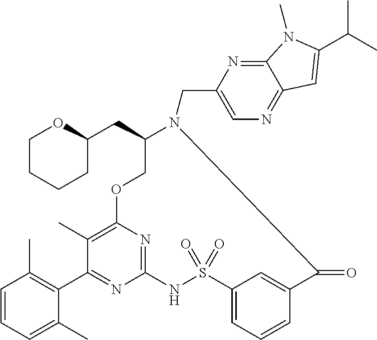 [SMILES: Cc1cccc(C)c1-c1nc2nc(c1C)OC[C@@H](C[C@H]1CCCCO1)N(Cc1cnc3cc(C(C)C)n(C)c3n1)C(=O)c1cccc(c1)S(=O)(=O)N2] | 1.64 | 723.32 | 724.2 | A |
| I-195 | 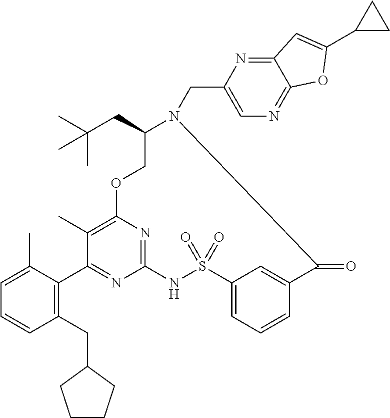 [SMILES: Cc1cccc(CC2CCCC2)c1-c1nc2nc(c1C)OC[C@@H](CC(C)(C)C)N(Cc1cnc3oc(C4CC4)cc3n1)C(=O)c1cccc(c1)S(=O)(=O)N2] | 2.52 | 748.341 | 749.3 | A |
| I-198 | 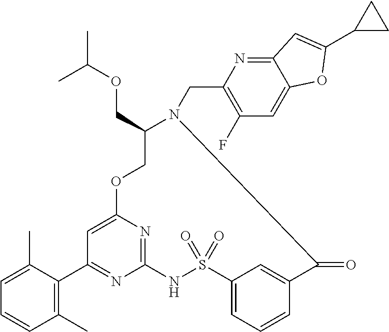 [SMILES: Cc1cccc(C)c1-c1cc2nc(n1)NS(=O)(=O)c1cccc(c1)C(=O)N(Cc1nc3cc(C4CC4)oc3cc1F)[C@H](COC(C)C)CO2] | 2.02 | 685.237 | 686.9 | A |
| I-199 | 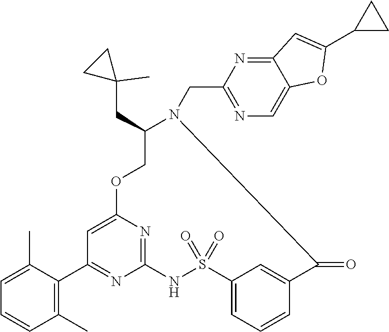 [SMILES: Cc1cccc(C)c1-c1cc2nc(n1)NS(=O)(=O)c1cccc(c1)C(=O)N(Cc1ncc3oc(C4CC4)cc3n1)[C@H](CC1(C)CC1)CO2] | 1.81 | 664.247 | 665.7 | A |
| I-200 | 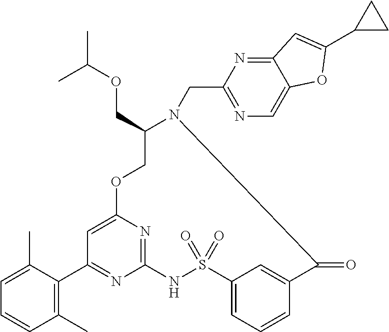 [SMILES: Cc1cccc(C)c1-c1cc2nc(n1)NS(=O)(=O)c1cccc(c1)C(=O)N(Cc1ncc3oc(C4CC4)cc3n1)[C@H](COC(C)C)CO2] | 1.76 | 668.242 | 669.6 | A |
| I-204 | 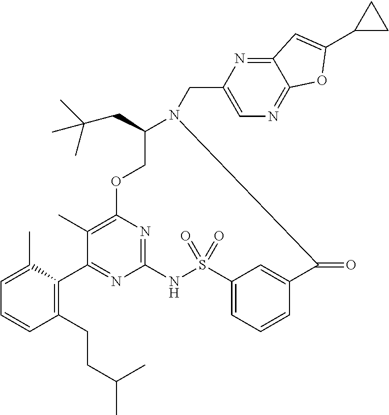 | 2.27 | 736.341 | 737.2 | A |
| I-205 | 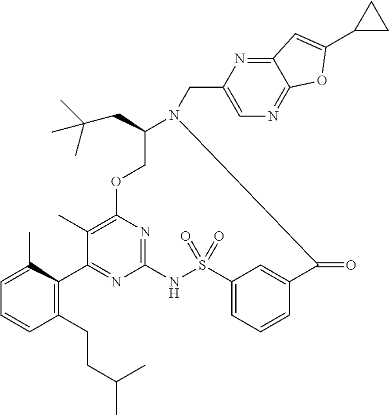 | 2.23 | 736.341 | 737.2 | A |
| I-207 | 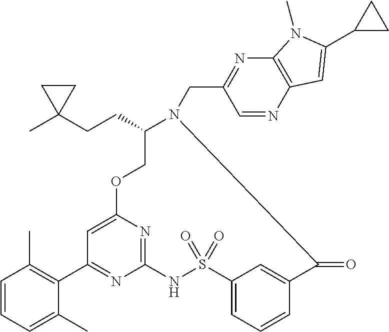 [SMILES: Cc1cccc(C)c1-c1cc2nc(n1)NS(=O)(=O)c1cccc(c1)C(=O)N(Cc1cnc3cc(C4CC4)n(C)c3n1)[C@@H](CCC1(C)CC1)CO2] | 1.73 | 691.294 | 692.2 | A |
| I-208 | 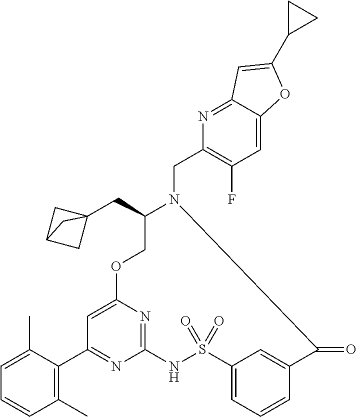 [SMILES: Cc1cccc(C)c1-c1cc2nc(n1)NS(=O)(=O)c1cccc(c1)C(=O)N(Cc1nc3cc(C4CC4)oc3cc1F)[C@H](CC13CC(C1)C3)CO2] | 2.14 | 693.242 | 694.6 | A |
| I-209 | 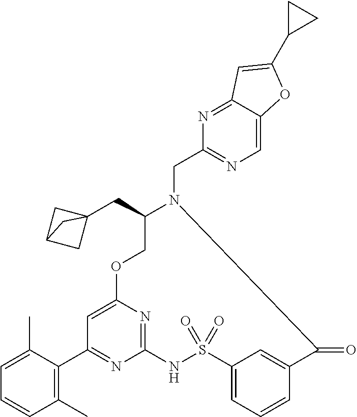 [SMILES: Cc1cccc(C)c1-c1cc2nc(n1)NS(=O)(=O)c1cccc(c1)C(=O)N(Cc1ncc3oc(C4CC4)cc3n1)[C@H](CC13CC(C1)C3)CO2] | 1.89 | 676.247 | 677.6 | A |
| I-211 | 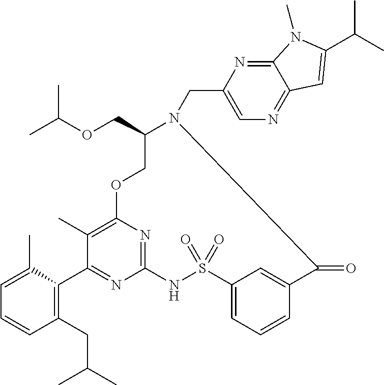 | 1.9 | 739.352 | 740.2 | A |
| I-212 | 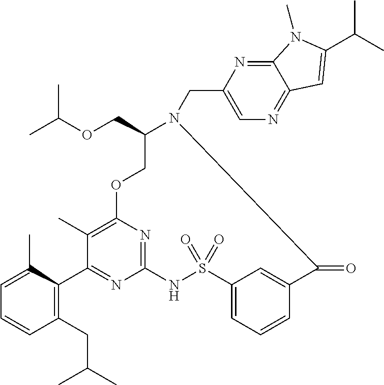 | 1.87 | 739.352 | 740.2 | A |
| I-213 | 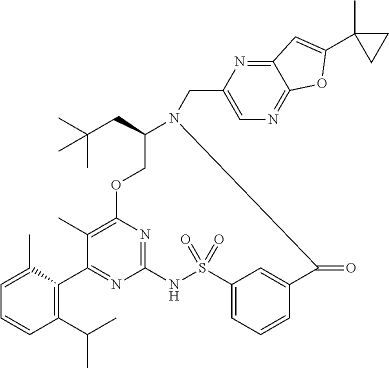 | 2.21 | 722.325 | 723.2 | A |
| I-214 | 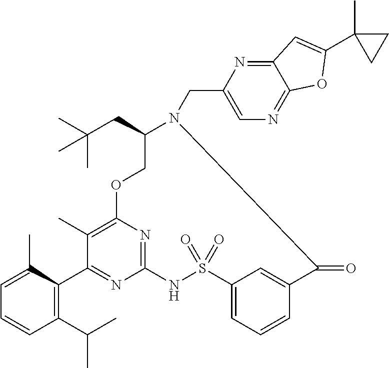 | 2.17 | 722.325 | 723.2 | A |
| I-215 | 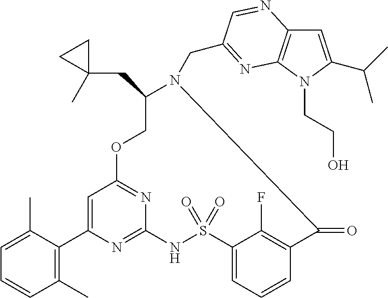 [SMILES: Cc1cccc(C)c1-c1cc2nc(n1)NS(=O)(=O)c1cccc(c1F)C(=O)N(Cc1cnc3cc(C(C)C)n(CCO)c3n1)[C@H](CC1(C)CC1)CO2] | 1.53 | 727.295 | 728.1 | A |
| I-216 | 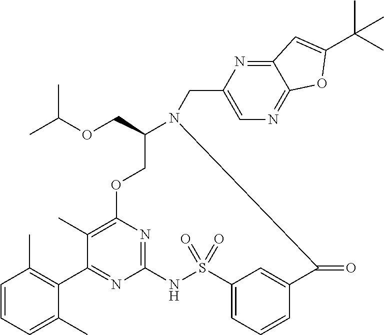 [SMILES: Cc1cccc(C)c1-c1nc2nc(c1C)OC[C@@H](COC(C)C)N(Cc1cnc3oc(C(C)(C)C)cc3n1)C(=O)c1cccc(c1)S(=O)(=O)N2] | 2.02 | 698.289 | 699.2 | A |
| I-217 | 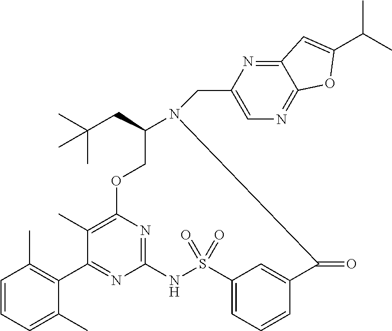 [SMILES: Cc1cccc(C)c1-c1nc2nc(c1C)OC[C@@H](CC(C)(C)C)N(Cc1cnc3oc(C(C)C)cc3n1)C(=O)c1cccc(c1)S(=O)(=O)N2] | 2.11 | 696.309 | 697.1 | A |
| I-218 | 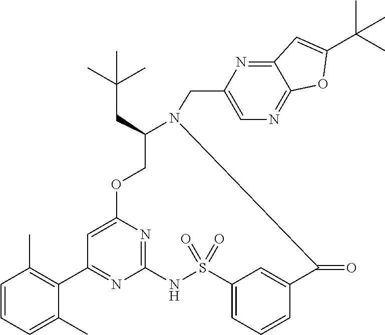 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC(C)(C)C)CO2] | 2.13 | 682.294 | 683.8 | A |
| I-219 | 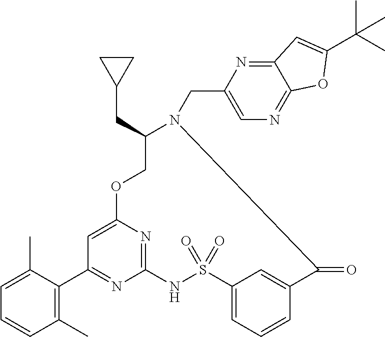 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CC1)CO2] | 2.01 | 666.262 | 667.8 | A |
| I-220 | 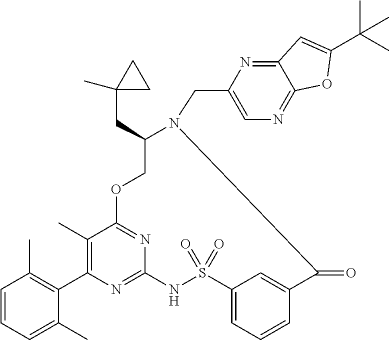 [SMILES: Cc1cccc(C)c1-c1nc2nc(c1C)OC[C@@H](CC1(C)CC1)N(Cc1cnc3oc(C(C)(C)C)cc3n1)C(=O)c1cccc(c1)S(=O)(=O)N2] | 2.12 | 694.294 | 695.7 | A |
| I-222 | 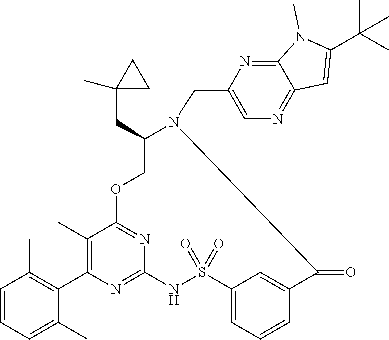 [SMILES: Cc1cccc(C)c1-c1nc2nc(c1C)OC[C@@H](CC1(C)CC1)N(Cc1cnc3cc(C(C)(C)C)n(C)c3n1)C(=O)c1cccc(c1)S(=O)(=O)N2] | 1.83 | 707.325 | 708.8 | A |
| I-223 | 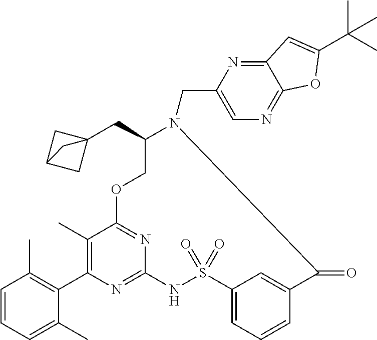 [SMILES: Cc1cccc(C)c1-c1nc2nc(c1C)OC[C@@H](CC13CC(C1)C3)N(Cc1cnc3oc(C(C)(C)C)cc3n1)C(=O)c1cccc(c1)S(=O)(=O)N2] | 2.02 | 706.294 | 707.7 | A |
| I-224 | 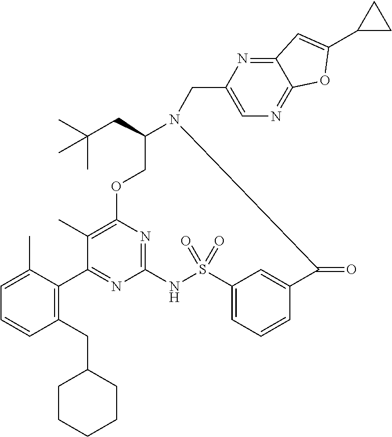 [SMILES: Cc1cccc(CC2CCCCC2)c1-c1nc2nc(c1C)OC[C@@H](CC(C)(C)C)N(Cc1cnc3oc(C4CC4)cc3n1)C(=O)c1cccc(c1)S(=O)(=O)N2] | 2.03 | 762.356 | 763.75 | C |
| I-226 | 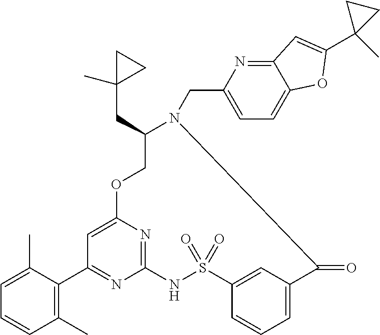 [SMILES: Cc1cccc(C)c1-c1cc2nc(n1)NS(=O)(=O)c1cccc(c1)C(=O)N(Cc1ccc3oc(C4(C)CC4)cc3n1)[C@H](CC1(C)CC1)CO2] | 1.85 | 677.267 | 678.8 | A |
| I-228 | 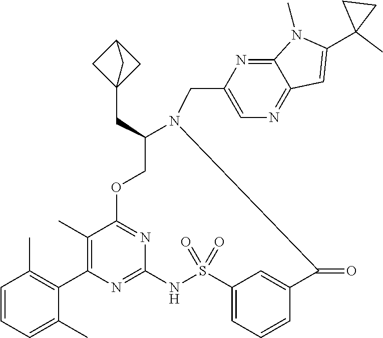 [SMILES: Cc1cccc(C)c1-c1nc2nc(c1C)OC[C@@H](CC13CC(C1)C3)N(Cc1cnc3cc(C4(C)CC4)n(C)c3n1)C(=O)c1cccc(c1)S(=O)(=O)N2] | 1.91 | 717.31 | 718.9 | A |
| I-229 | 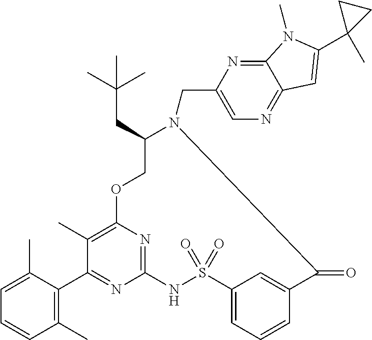 [SMILES: Cc1cccc(C)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] | 1.89 | 707.325 | 708.7 | A |
| I-230 | 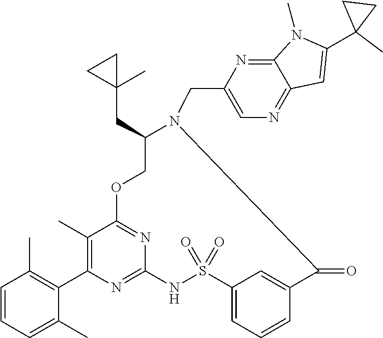 [SMILES: Cc1cccc(C)c1-c1nc2nc(c1C)OC[C@@H](CC1(C)CC1)N(Cc1cnc3cc(C4(C)CC4)n(C)c3n1)C(=O)c1cccc(c1)S(=O)(=O)N2] | 1.83 | 705.31 | 706.8 | A |
| I-231 | 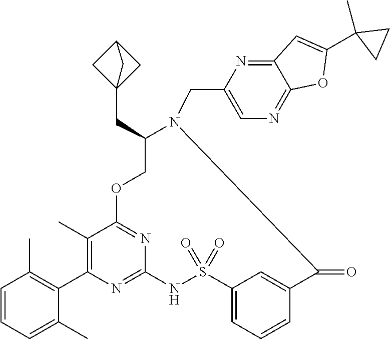 [SMILES: Cc1cccc(C)c1-c1nc2nc(c1C)OC[C@@H](CC13CC(C1)C3)N(Cc1cnc3oc(C4(C)CC4)cc3n1)C(=O)c1cccc(c1)S(=O)(=O)N2] | 2.16 | 704.278 | 705.7 | A |
| I-232 | 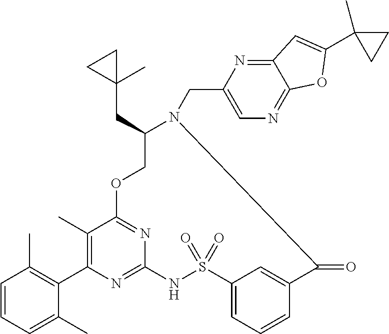 [SMILES: Cc1cccc(C)c1-c1nc2nc(c1C)OC[C@@H](CC1(C)CC1)N(Cc1cnc3oc(C4(C)CC4)cc3n1)C(=O)c1cccc(c1)S(=O)(=O)N2] | 2.07 | 692.278 | 693.9 | A |
| I-233 | 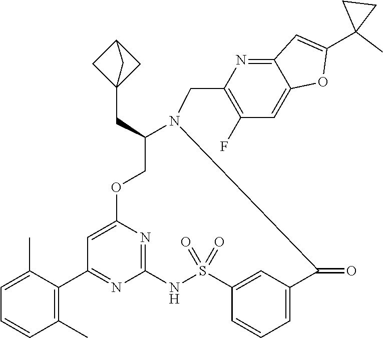 [SMILES: Cc1cccc(C)c1-c1cc2nc(n1)NS(=O)(=O)c1cccc(c1)C(=O)N(Cc1nc3cc(C4(C)CC4)oc3cc1F)[C@H](CC13CC(C1)C3)CO2] | 2.25 | 707.258 | 708.8 | A |
| I-235 | 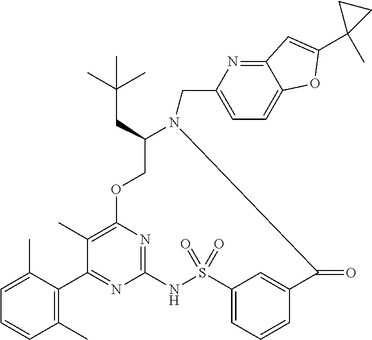 [SMILES: Cc1cccc(C)c1-c1nc2nc(c1C)OC[C@@H](CC(C)(C)C)N(Cc1ccc3oc(C4(C)CC4)cc3n1)C(=O)c1cccc(c1)S(=O)(=O)N2] | 1.95 | 693.298 | 694.9 | A |
| I-236 | 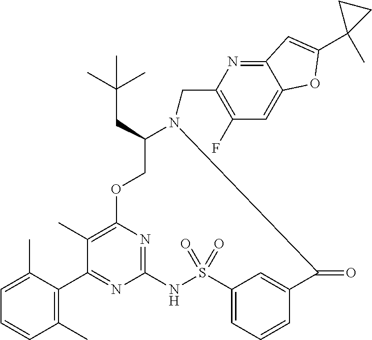 [SMILES: Cc1cccc(C)c1-c1nc2nc(c1C)OC[C@@H](CC(C)(C)C)N(Cc1nc3cc(C4(C)CC4)oc3cc1F)C(=O)c1cccc(c1)S(=O)(=O)N2] | 2.29 | 711.289 | 712.8 | A |
| I-238 | 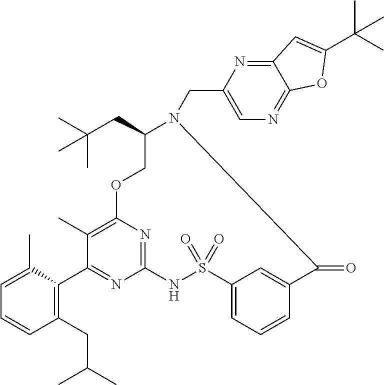 | 2.32 | 738.356 | 739.6 | A |
| I-239 | 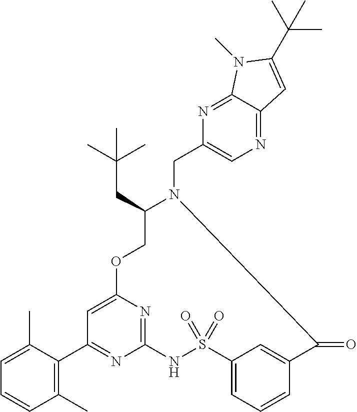 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](CC(C)(C)C)CO2] | 1.87 | 695.325 | 696.7 | A |
| I-240 | 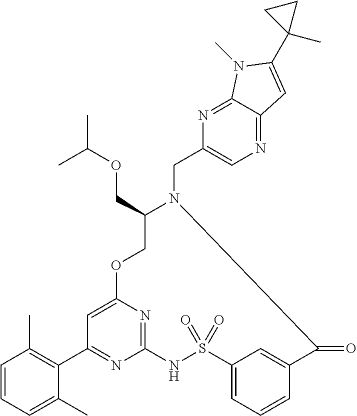 [SMILES: Cc1cccc(C)c1-c1cc2nc(n1)NS(=O)(=O)c1cccc(c1)C(=O)N(Cc1cnc3cc(C4(C)CC4)n(C)c3n1)[C@H](COC(C)C)CO2] | 1.75 | 695.289 | 696.7 | A |
| I-241 | 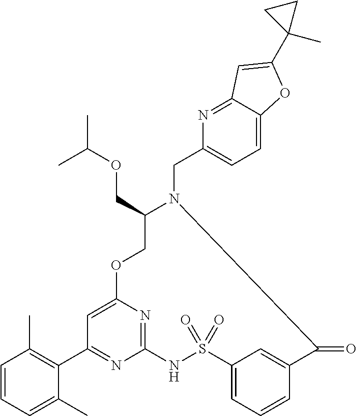 [SMILES: Cc1cccc(C)c1-c1cc2nc(n1)NS(=O)(=O)c1cccc(c1)C(=O)N(Cc1ccc3oc(C4(C)CC4)cc3n1)[C@H](COC(C)C)CO2] | 1.69 | 681.262 | 682.7 | A |
| I-242 | 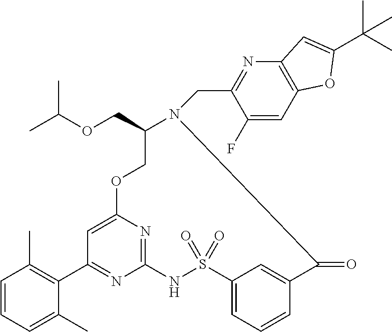 [SMILES: Cc1cccc(C)c1-c1cc2nc(n1)NS(=O)(=O)c1cccc(c1)C(=O)N(Cc1nc3cc(C(C)(C)C)oc3cc1F)[C@H](COC(C)C)CO2] | 2.11 | 701.268 | 702.1 | A |
| I-243 | 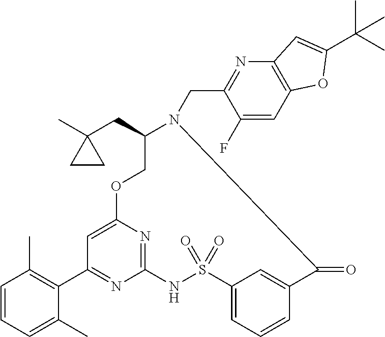 [SMILES: Cc1cccc(C)c1-c1cc2nc(n1)NS(=O)(=O)c1cccc(c1)C(=O)N(Cc1nc3cc(C(C)(C)C)oc3cc1F)[C@H](CC1(C)CC1)CO2] | 2.14 | 697.273 | 698.1 | A |
| I-244 | 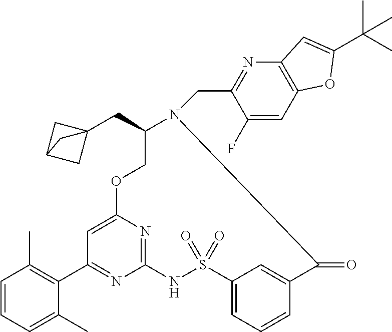 [SMILES: Cc1cccc(C)c1-c1cc2nc(n1)NS(=O)(=O)c1cccc(c1)C(=O)N(Cc1nc3cc(C(C)(C)C)oc3cc1F)[C@H](CC13CC(C1)C3)CO2] | 2.22 | 709.273 | 710.2 | A |
| I-245 | 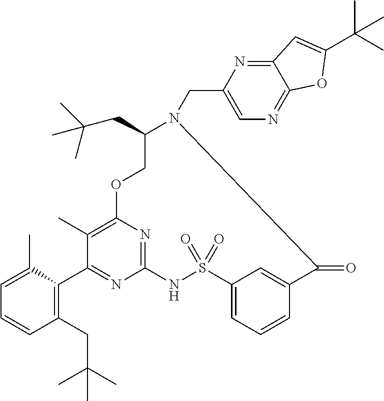 | 2.36 | 752.372 | 753.2 | A |
| I-246 | 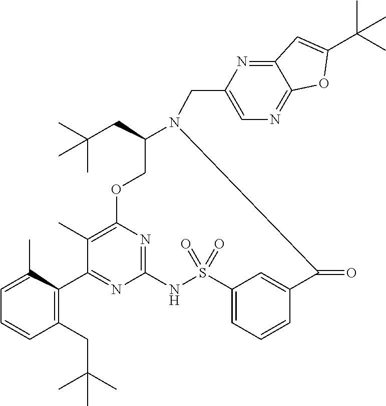 | 2.32 | 752.372 | 753.6 | A |
| I-247 | 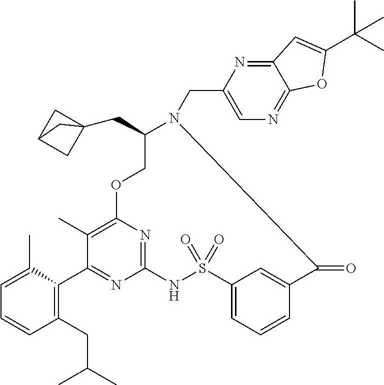 | 2.32 | 748.341 | 749.2 | A |
| I-248 | 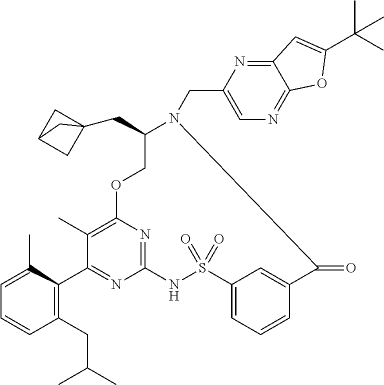 | 2.31 | 748.341 | 749.2 | A |
| I-249 | 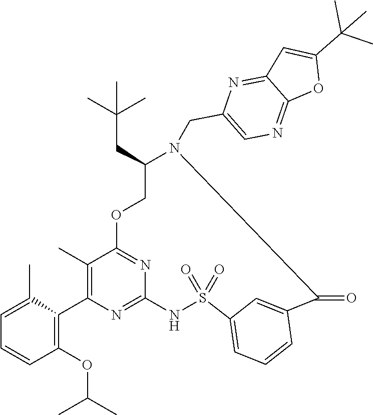 | 2.29 | 740.336 | 741.8 | A |
| I-250 | 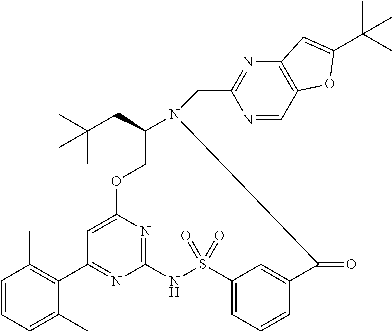 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC(C)(C)C)CO2] | 1.98 | 682.294 | 683.2 | A |
| I-251 | 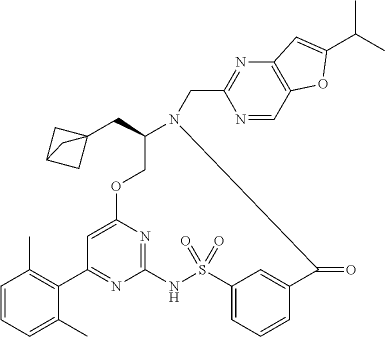 [SMILES: Cc1cccc(C)c1-c1cc2nc(n1)NS(=O)(=O)c1cccc(c1)C(=O)N(Cc1ncc3oc(C(C)C)cc3n1)[C@H](CC13CC(C1)C3)CO2] | 2 | 692.278 | 693.1 | A |
| I-252 | 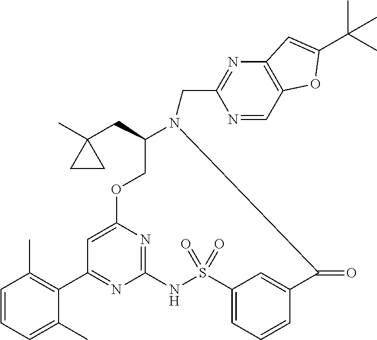 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC1(C)CC1)CO2] | 1.93 | 680.278 | 681.3 | A |
| I-253 | 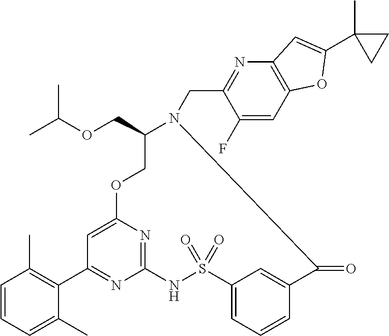 [SMILES: Cc1cccc(C)c1-c1cc2nc(n1)NS(=O)(=O)c1cccc(c1)C(=O)N(Cc1nc3cc(C4(C)CC4)oc3cc1F)[C@H](COC(C)C)CO2] | 2.06 | 699.253 | 700.1 | A |
| I-254 | 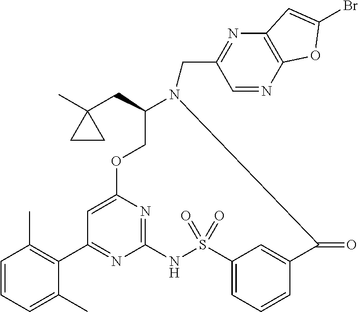 [SMILES: Cc1cccc(C)c1-c1cc2nc(n1)NS(=O)(=O)c1cccc(c1)C(=O)N(Cc1cnc3oc(Br)cc3n1)[C@H](CC1(C)CC1)CO2] | 1.84 | 702.126 | 704.9 (M + 3) | A |
| I-255 | 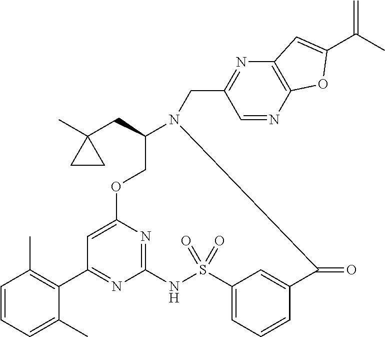 [SMILES: C=C(C)c1cc2nc(CN3C(=O)c4cccc(c4)S(=O)(=O)Nc4nc(cc(-c5c(C)cccc5C)n4)OC[C@H]3CC3(C)CC3)cnc2o1] | 1.91 | 664.247 | 665.1 | A |
| I-256 | 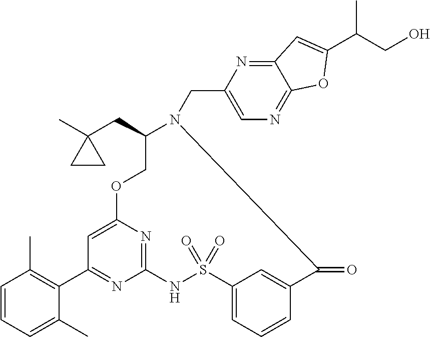 [SMILES: Cc1cccc(C)c1-c1cc2nc(n1)NS(=O)(=O)c1cccc(c1)C(=O)N(Cc1cnc3oc(C(C)CO)cc3n1)[C@H](CC1(C)CC1)CO2] | 1.43 | 682.257 | 683.61 | A |
| I-257 | 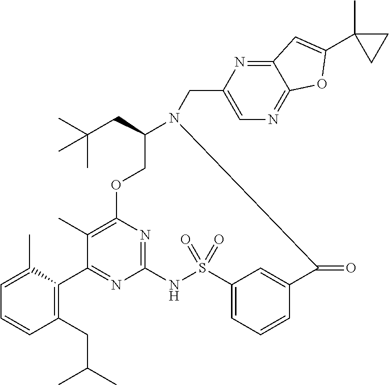 | 2.28 | 736.341 | 737.9 | A |
| 1-258 | 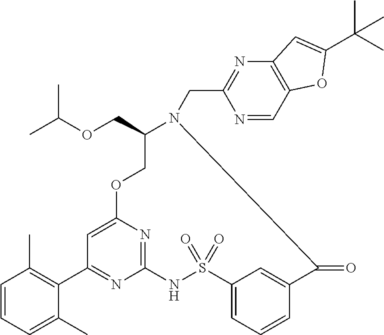 [SMILES: Cc1cccc(C)c1-c1cc2nc(n1)NS(=O)(=O)c1cccc(c1)C(=O)N(Cc1ncc3oc(C(C)(C)C)cc3n1)[C@H](COC(C)C)CO2] | 1.89 | 684.273 | 685.6 | A |
| I-259 | 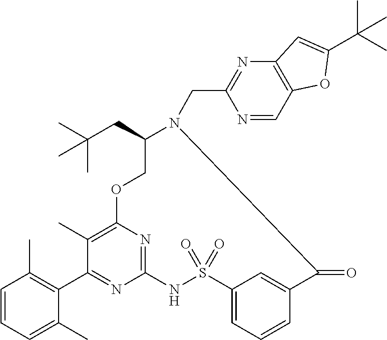 [SMILES: Cc1cccc(C)c1-c1nc2nc(c1C)OC[C@@H](CC(C)(C)C)N(Cc1ncc3oc(C(C)(C)C)cc3n1)C(=O)c1cccc(c1)S(=O)(=O)N2] | 2.04 | 696.309 | 697.7 | A |
| I-260 | 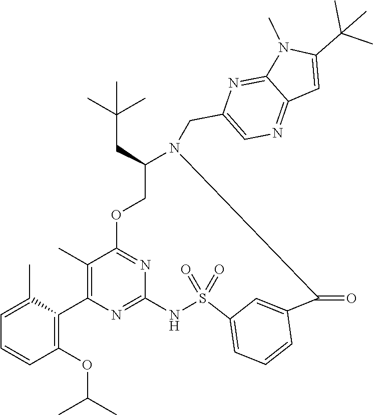 | 1.95 | 753.367 | 754.7 | A |
| I-261 | 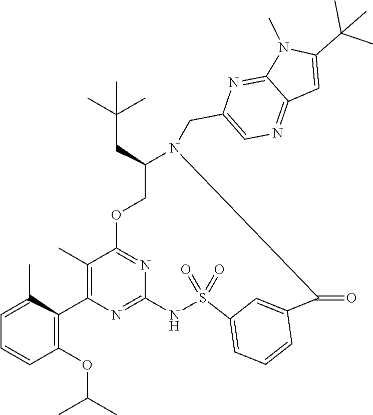 | 1.92 | 753.367 | 754.6 | A |
| I-262 | 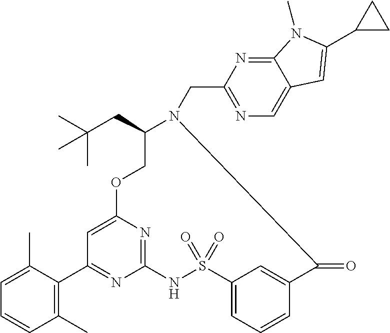 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC(C)(C)C)CO2] | 1.31 | 679.294 | 680.6 | A |
| I-263 | 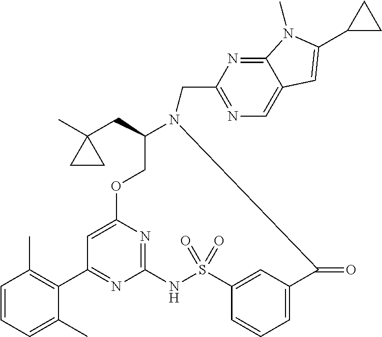 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC1(C)CC1)CO2] | 1.27 | 677.278 | 678.5 | A |
| I-264 | 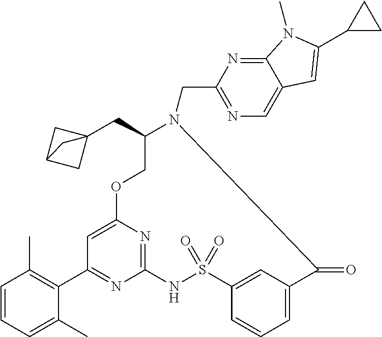 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC13CC(C1)C3)CO2] | 1.3 | 689.278 | 690.5 | A |
| I-265 | 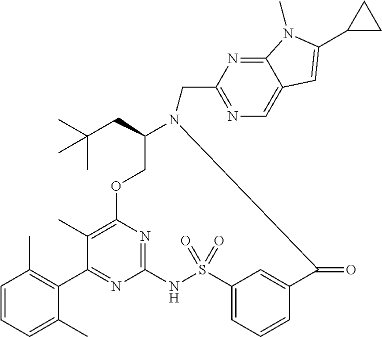 [SMILES: Cc1cccc(C)c1-c1nc2nc(c1C)OC[C@@H](CC(C)(C)C)N(Cc1ncc3cc(C4CC4)n(C)c3n1)C(=O)c1cccc(c1)S(=O)(=O)N2] | 1.33 | 693.31 | 694.6 | A |
| II-1 | 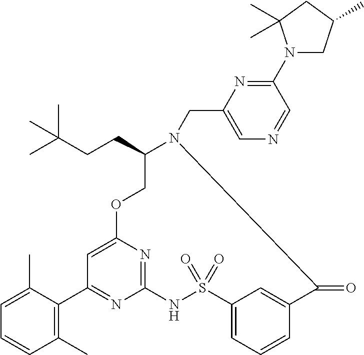 [SMILES: Cc1cccc(C)c1-c1cc2nc(n1)NS(=O)(=O)c1cccc(c1)C(=O)N(Cc1cncc(N3C[C@@H](C)CC3(C)C)n1)[C@H](CCC(C)(C)C)CO2] | 2 | 711.357 | 712.2 | A |
| II-3 | 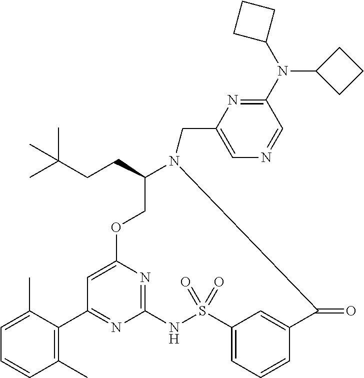 [SMILES: Cc1cccc(C)c1-c1cc2nc(n1)NS(=O)(=O)c1cccc(c1)C(=O)N(Cc1cncc(N(C3CCC3)C3CCC3)n1)[C@H](CCC(C)(C)C)CO2] | 2.09 | 723.357 | 724.2 | A |
| II-4 | 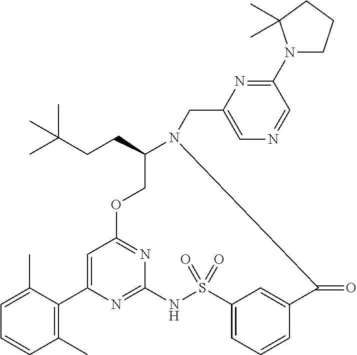 [SMILES: Cc1cccc(C)c1-c1cc2nc(n1)NS(=O)(=O)c1cccc(c1)C(=O)N(Cc1cncc(N3CCCC3(C)C)n1)[C@H](CCC(C)(C)C)CO2] | 1.91 | 697.341 | 698.2 | A |
| II-5 | 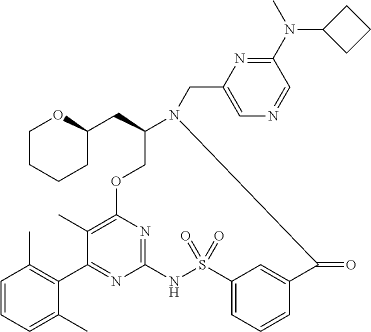 [SMILES: Cc1cccc(C)c1-c1nc2nc(c1C)OC[C@@H](C[C@H]1CCCCO1)N(Cc1cncc(N(C)C3CCC3)n1)C(=O)c1cccc(c1)S(=O)(=O)N2] | 1.76 | 697.305 | 698.9 | A |
| II-6 | 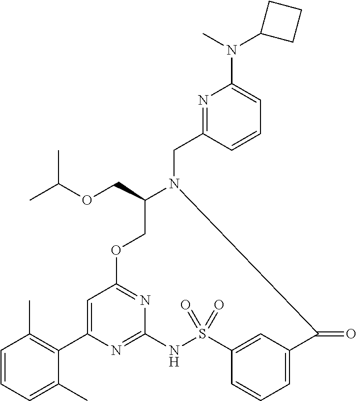 [SMILES: Cc1cccc(C)c1-c1cc2nc(n1)NS(=O)(=O)c1cccc(c1)C(=O)N(Cc1cccc(N(C)C3CCC3)n1)[C@H](COC(C)C)CO2] | 1.19 | 670.294 | 671.5 | A |
| II-8 | 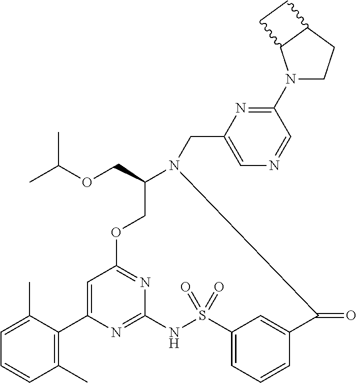 [SMILES: Cc1cccc(C)c1-c1cc2nc(n1)NS(=O)(=O)c1cccc(c1)C(=O)N(Cc1cncc(N3CCC4CCC43)n1)[C@H](COC(C)C)CO2] | 1.8 | 683.289 | 684.9 | A |
| II-9 | 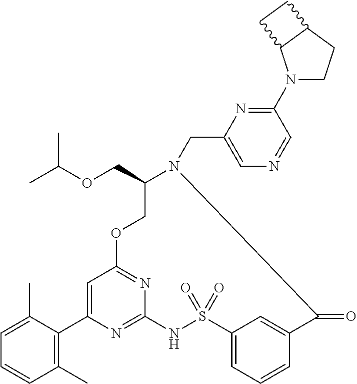 [SMILES: Cc1cccc(C)c1-c1cc2nc(n1)NS(=O)(=O)c1cccc(c1)C(=O)N(Cc1cncc(N3CCC4CCC43)n1)[C@H](COC(C)C)CO2] | 1.78 | 683.289 | 684.7 | A |
| II-10 | 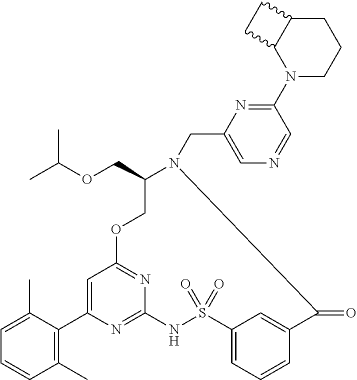 [SMILES: Cc1cccc(C)c1-c1cc2nc(n1)NS(=O)(=O)c1cccc(c1)C(=O)N(Cc1cncc(N3CCCC4CCC43)n1)[C@H](COC(C)C)CO2] | 1.88 | 697.305 | 698.9 | A |
| II-11 | 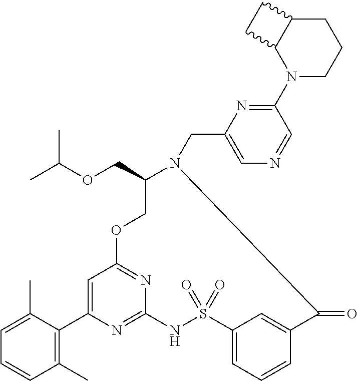 [SMILES: Cc1cccc(C)c1-c1cc2nc(n1)NS(=O)(=O)c1cccc(c1)C(=O)N(Cc1cncc(N3CCCC4CCC43)n1)[C@H](COC(C)C)CO2] | 1.89 | 697.305 | 698.7 | A |
| II-12 | 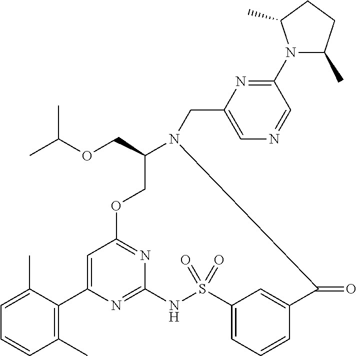 [SMILES: Cc1cccc(C)c1-c1cc2nc(n1)NS(=O)(=O)c1cccc(c1)C(=O)N(Cc1cncc(N3[C@H](C)CC[C@H]3C)n1)[C@H](COC(C)C)CO2] | 1.77 | 685.305 | 686.9 | A |
| II-13 | 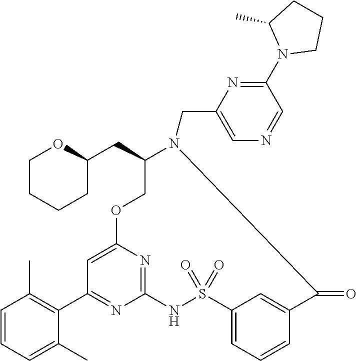 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@H]1CCCCO1)CO2] | 1.68 | 697.305 | 698.9 | A |
| II-14 | 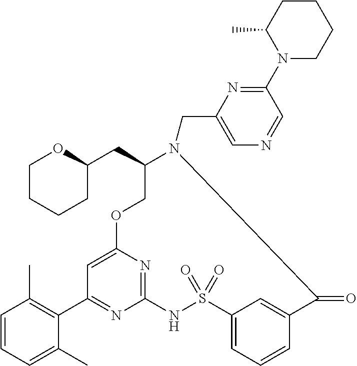 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@H]1CCCCO1)CO2] | 1.77 | 711.32 | 712.9 | A |
| II-15 | 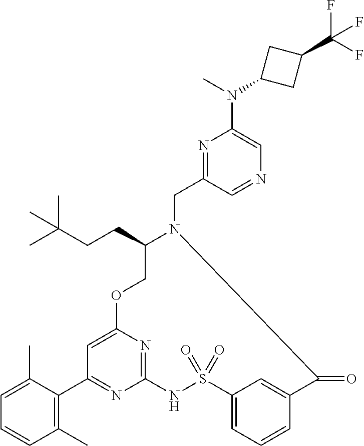 [SMILES: Cc1cccc(C)c1-c1cc2nc(n1)NS(=O)(=O)c1cccc(c1)C(=O)N(Cc1cncc(N(C)[C@H]3C[C@H](C(F)(F)F)C3)n1)[C@H](CCC(C)(C)C)CO2] | 1.75 | 751.313 | 752.5 | A |
| II-16 | 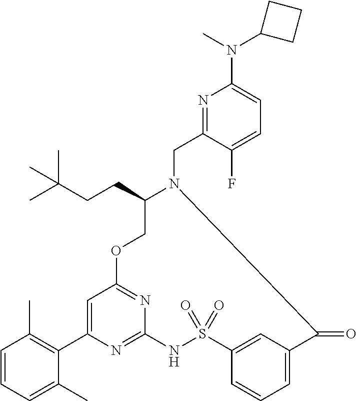 [SMILES: Cc1cccc(C)c1-c1cc2nc(n1)NS(=O)(=O)c1cccc(c1)C(=O)N(Cc1nc(N(C)C3CCC3)ccc1F)[C@H](CCC(C)(C)C)CO2] | 1.82 | 700.321 | 701.5 | A |
| II-17 | 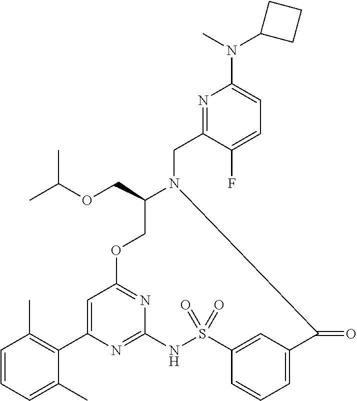 [SMILES: Cc1cccc(C)c1-c1cc2nc(n1)NS(=O)(=O)c1cccc(c1)C(=O)N(Cc1nc(N(C)C3CCC3)ccc1F)[C@H](COC(C)C)CO2] | 1.59 | 688.284 | 689.4 | A |
| II-18 | 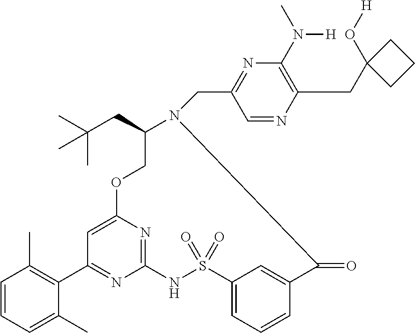 [SMILES: CNc1nc(CN2C(=O)c3cccc(c3)S(=O)(=O)Nc3nc(cc(-c4c(C)cccc4C)n3)OC[C@H]2CC(C)(C)C)cnc1CC1(O)CCC1] | 1.63 | 699.32 | 700.3 | A |
| II-19 | 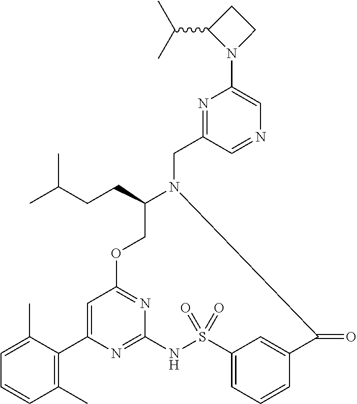 [SMILES: Cc1cccc(C)c1-c1cc2nc(n1)NS(=O)(=O)c1cccc(c1)C(=O)N(Cc1cncc(N3CCC3C(C)C)n1)[C@H](CCC(C)C)CO2] | 1.74 | 683.325 | 684.5 | A |
| II-20 | 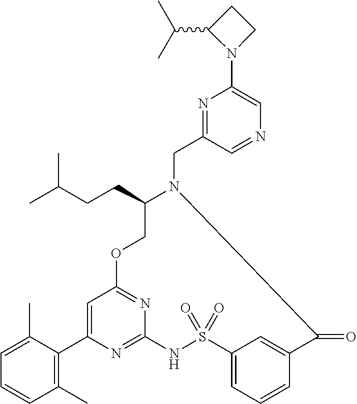 [SMILES: Cc1cccc(C)c1-c1cc2nc(n1)NS(=O)(=O)c1cccc(c1)C(=O)N(Cc1cncc(N3CCC3C(C)C)n1)[C@H](CCC(C)C)CO2] | 1.75 | 683.325 | 684.4 | A |
| II-21 | 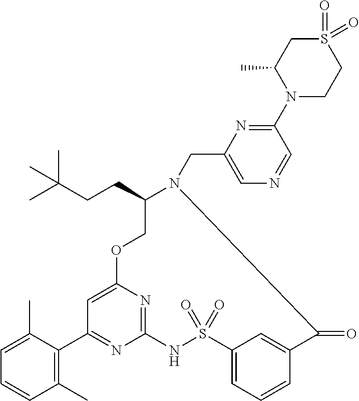 [SMILES: Cc1cccc(C)c1-c1cc2nc(n1)NS(=O)(=O)c1cccc(c1)C(=O)N(Cc1cncc(N3CCS(=O)(=O)C[C@H]3C)n1)[C@H](CCC(C)(C)C)CO2] | 1.88 | 747.287 | 748.4 | A |
| II-22 | 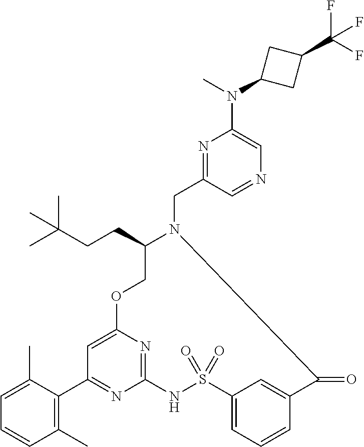 [SMILES: Cc1cccc(C)c1-c1cc2nc(n1)NS(=O)(=O)c1cccc(c1)C(=O)N(Cc1cncc(N(C)[C@H]3C[C@@H](C(F)(F)F)C3)n1)[C@H](CCC(C)(C)C)CO2] | 1.78 | 751.313 | 752.6 | A |
| II-23 | 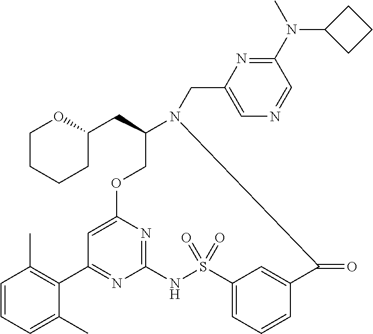 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCCO1)CO2] | 1.81 | 697.305 | 698.7 | A |
| II-24 | 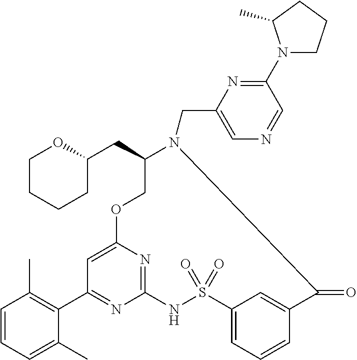 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@@H]1CCCCO1)CO2] | 1.73 | 697.305 | 698.9 | A |
| II-25 | 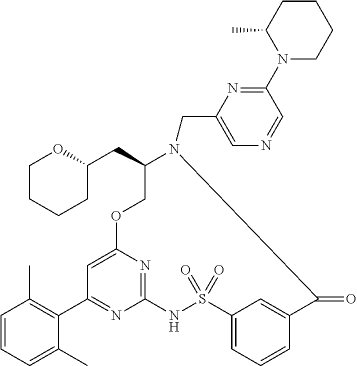 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@@H]1CCCCO1)CO2] | 1.82 | 711.32 | 712.9 | A |
| II-26 | 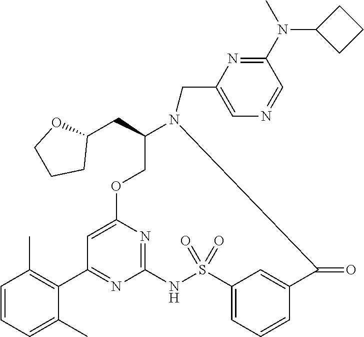 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCO1)CO2] | 1.67 | 683.289 | 684.7 | A |
| II-27 | 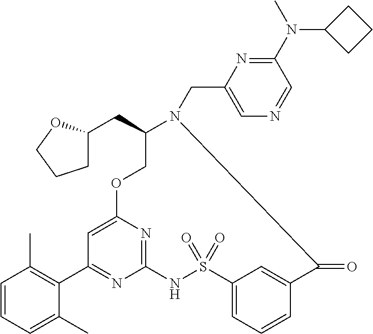 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCO1)CO2] | 1.69 | 683.289 | 684.9 | A |
| II-28 | 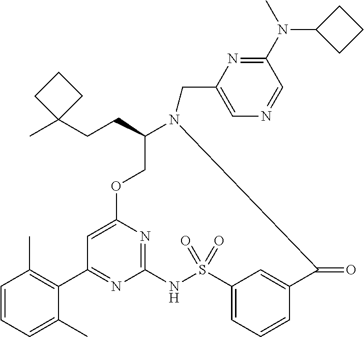 [SMILES: Cc1cccc(C)c1-c1cc2nc(n1)NS(=O)(=O)c1cccc(c1)C(=O)N(Cc1cncc(N(C)C3CCC3)n1)[C@H](CCC1(C)CCC1)CO2] | 2.09 | 695.325 | 696.8 | A |
| II-29 | 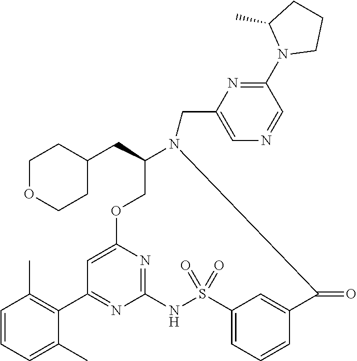 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CCOCC1)CO2] | 1.57 | 697.305 | 698.8 | A |
| II-30 | 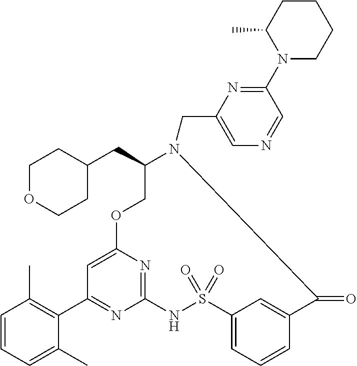 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CCOCC1)CO2] | 1.68 | 711.32 | 712.9 | A |
| II-31 | 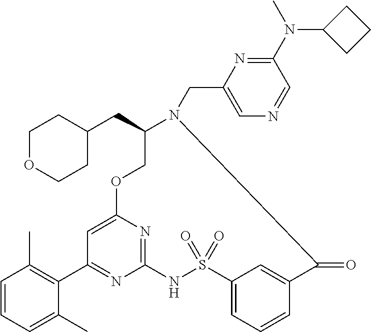 [SMILES: Cc1cccc(C)c1-c1cc2nc(n1)NS(=O)(=O)c1cccc(c1)C(=O)N(Cc1cncc(N(C)C3CCC3)n1)[C@H](CC1CCOCC1)CO2] | 1.64 | 697.305 | 698.9 | A |
| II-32 | 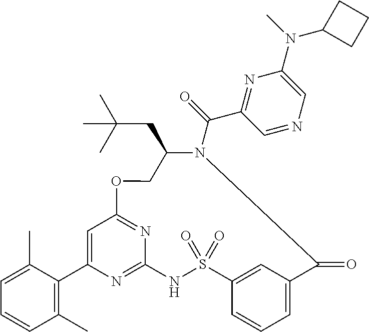 [SMILES: Cc1cccc(C)c1-c1cc2nc(n1)NS(=O)(=O)c1cccc(c1)C(=O)N(C(=O)c1cncc(N(C)C3CCC3)n1)[C@H](CC(C)(C)C)CO2] | 2.1 | 683.289 | 684.2 | A |
| II-33 | 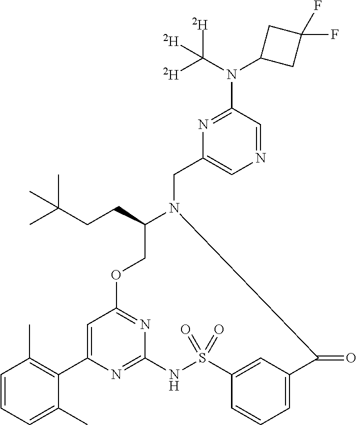 [SMILES: [2H]C([2H])([2H])N(c1cncc(CN2C(=O)c3cccc(c3)S(=O)(=O)Nc3nc(cc(-c4c(C)cccc4C)n3)OC[C@H]2CCC(C)(C)C)n1)C1CC(F)(F)C1] | 1.65 | 722.325 | 723.6 | A |
| II-34 | 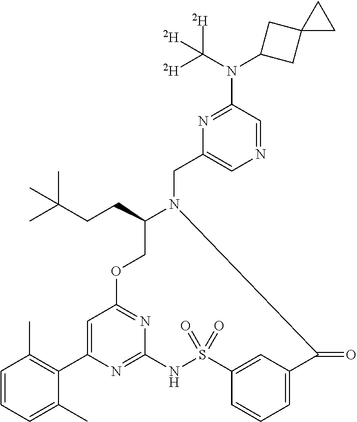 [SMILES: [2H]C([2H])([2H])N(c1cncc(CN2C(=O)c3cccc(c3)S(=O)(=O)Nc3nc(cc(-c4c(C)cccc4C)n3)OC[C@H]2CCC(C)(C)C)n1)C1CC2(CC2)C1] | 1.73 | 712.36 | 713.6 | A |
| II-35 | 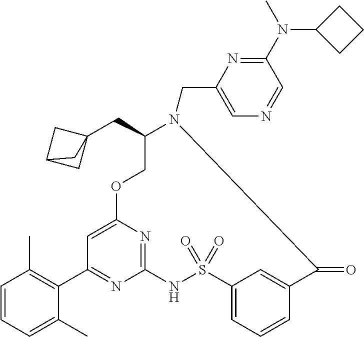 [SMILES: Cc1cccc(C)c1-c1cc2nc(n1)NS(=O)(=O)c1cccc(c1)C(=O)N(Cc1cncc(N(C)C3CCC3)n1)[C@H](CC13CC(C1)C3)CO2] | 1.97 | 679.294 | 680.4 | A |
| II-36 | 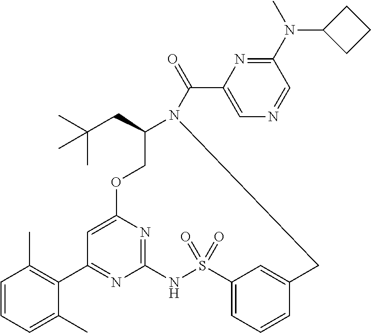 [SMILES: Cc1cccc(C)c1-c1cc2nc(n1)NS(=O)(=O)c1cccc(c1)CN(C(=O)c1cncc(N(C)C3CCC3)n1)[C@H](CC(C)(C)C)CO2] | 2.14 | 669.31 | 670.2 | A |
| II-37 | 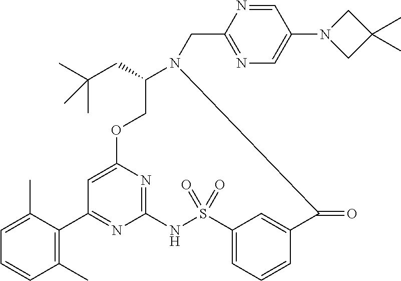 [SMILES: Cc1cccc(C)c1-c1cc2nc(n1)NS(=O)(=O)c1cccc(c1)C(=O)N(Cc1ncc(N3CC(C)(C)C3)cn1)[C@@H](CC(C)(C)C)CO2] | 2.65 | 669.31 | 670.5 | A (5 min gradient) |
| II-38 | 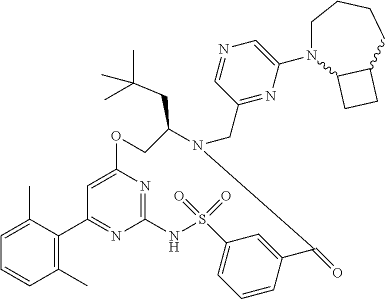 [SMILES: Cc1cccc(C)c1-c1cc2nc(n1)NS(=O)(=O)c1cccc(c1)C(=O)N(Cc1cncc(N3CCCCC4CCC43)n1)[C@H](CC(C)(C)C)CO2] | 4.96 | 709.341 | 710.1 | J |
| III-4 | 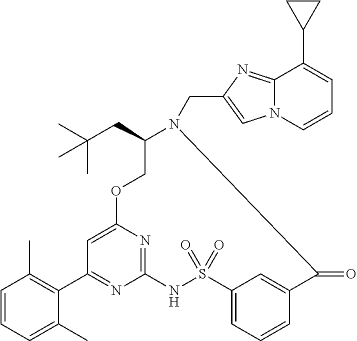 [SMILES: Cc1cccc(C)c1-c1cc2nc(n1)NS(=O)(=O)c1cccc(c1)C(=O)N(Cc1cn3cccc(C4CC4)c3n1)[C@H](CC(C)(C)C)CO2] | 1.48 | 664.283 | 665.2 | A |
| III-6 | 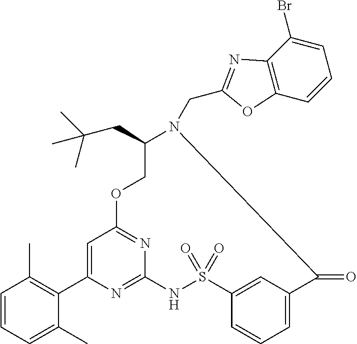 [SMILES: Cc1cccc(C)c1-c1cc2nc(n1)NS(=O)(=O)c1cccc(c1)C(=O)N(Cc1nc3c(Br)cccc3o1)[C@H](CC(C)(C)C)CO2] | 4.65 | 703.146 | 704.3 | J |
| III-7 | 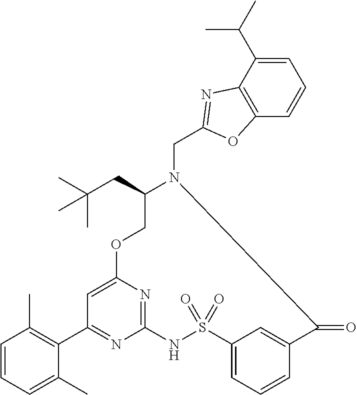 [SMILES: Cc1cccc(C)c1-c1cc2nc(n1)NS(=O)(=O)c1cccc(c1)C(=O)N(Cc1nc3c(C(C)C)cccc3o1)[C@H](CC(C)(C)C)CO2] | 4.94 | 667.283 | 668.4 | J |
| III-8 | 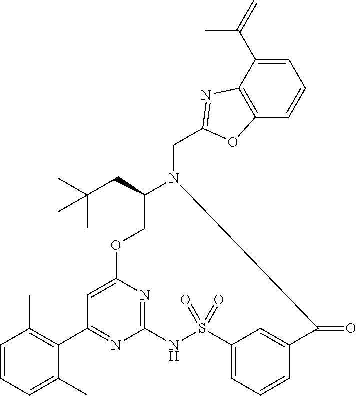 [SMILES: C=C(C)c1cccc2oc(CN3C(=O)c4cccc(c4)S(=O)(=O)Nc4nc(cc(-c5c(C)cccc5C)n4)OC[C@H]3CC(C)(C)C)nc12] | 4.85 | 665.267 | 666.3 | J |
| III-9 | 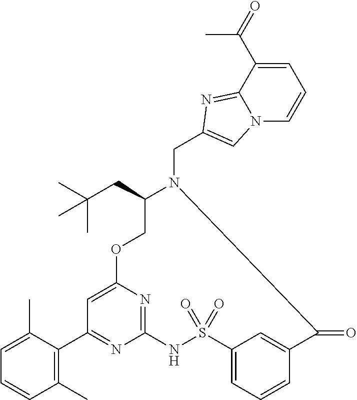 [SMILES: CC(=O)c1cccn2cc(CN3C(=O)c4cccc(c4)S(=O)(=O)Nc4nc(cc(-c5c(C)cccc5C)n4)OC[C@H]3CC(C)(C)C)nc12] | 1.4 | 666.262 | 667.2 | A |
| III-10 | 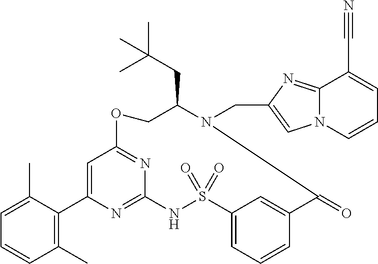 [SMILES: Cc1cccc(C)c1-c1cc2nc(n1)NS(=O)(=O)c1cccc(c1)C(=O)N(Cc1cn3cccc(C#N)c3n1)[C@H](CC(C)(C)C)CO2] | 1.78 | 649.247 | 650.4 | A |
| III-11 | 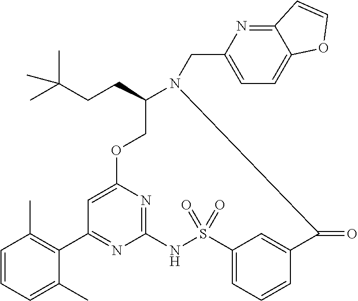 [SMILES: Cc1cccc(C)c1-c1cc2nc(n1)NS(=O)(=O)c1cccc(c1)C(=O)N(Cc1ccc3occc3n1)[C@H](CCC(C)(C)C)CO2] | 1.99 | 639.252 | 640.4 | A |
| III-12 | 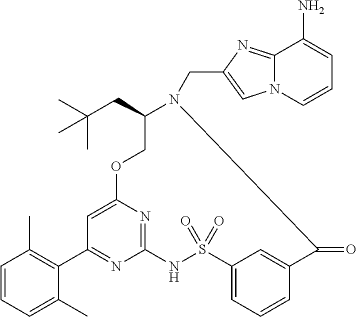 [SMILES: Cc1cccc(C)c1-c1cc2nc(n1)NS(=O)(=O)c1cccc(c1)C(=O)N(Cc1cn3cccc(N)c3n1)[C@H](CC(C)(C)C)CO2] | 1.39 | 639.263 | 640.2 | A |
| III-15 | 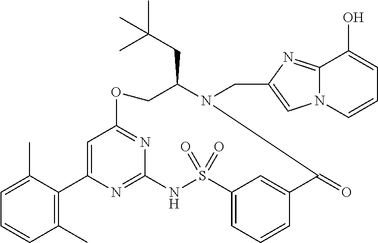 [SMILES: Cc1cccc(C)c1-c1cc2nc(n1)NS(=O)(=O)c1cccc(c1)C(=O)N(Cc1cn3cccc(O)c3n1)[C@H](CC(C)(C)C)CO2] | 1.39 | 640.247 | 641.4 | A |
| III-17 | 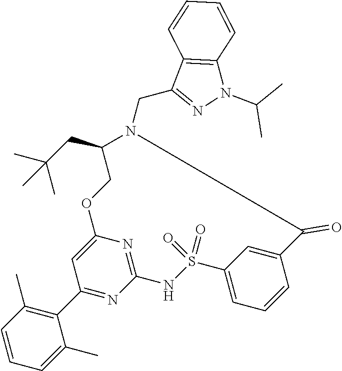 [SMILES: Cc1cccc(C)c1-c1cc2nc(n1)NS(=O)(=O)c1cccc(c1)C(=O)N(Cc1nn(C(C)C)c3ccccc13)[C@H](CC(C)(C)C)CO2] | 2.23 | 666.299 | 667.9 | A |
| III-18 | 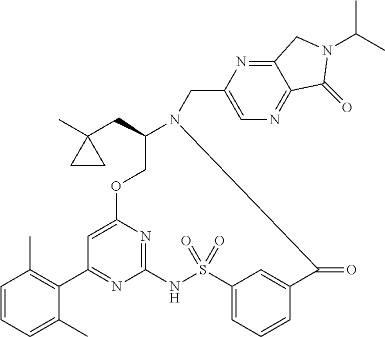 [SMILES: Cc1cccc(C)c1-c1cc2nc(n1)NS(=O)(=O)c1cccc(c1)C(=O)N(Cc1cnc3c(n1)CN(C(C)C)C3=O)[C@H](CC1(C)CC1)CO2] | 1.61 | 681.273 | 682.2 | A |
| III-19 | 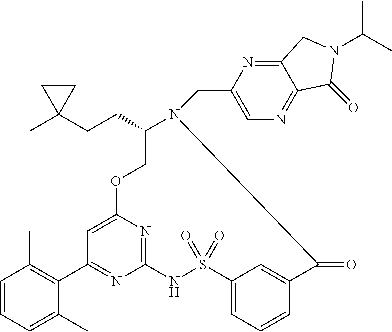 [SMILES: Cc1cccc(C)c1-c1cc2nc(n1)NS(=O)(=O)c1cccc(c1)C(=O)N(Cc1cnc3c(n1)CN(C(C)C)C3=O)[C@@H](CCC1(C)CC1)CO2] | 1.72 | 695.289 | 696.4 | A |
| III-22 | 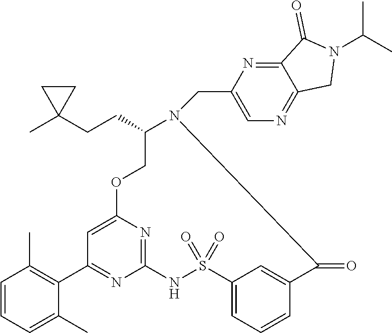 [SMILES: Cc1cccc(C)c1-c1cc2nc(n1)NS(=O)(=O)c1cccc(c1)C(=O)N(Cc1cnc3c(n1)C(=O)N(C(C)C)C3)[C@@H](CCC1(C)CC1)CO2] | 1.72 | 695.289 | 696.8 | A |
| III-23 | 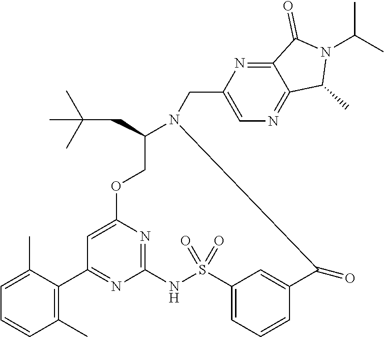 [SMILES: Cc1cccc(C)c1-c1cc2nc(n1)NS(=O)(=O)c1cccc(c1)C(=O)N(Cc1cnc3c(n1)C(=O)N(C(C)C)[C@@H]3C)[C@H](CC(C)(C)C)CO2] | 4.12 | 697.305 | 698.3 | J |
| III-24 | 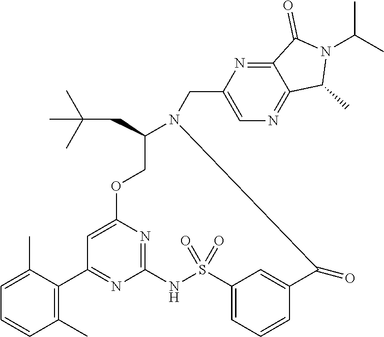 [SMILES: Cc1cccc(C)c1-c1cc2nc(n1)NS(=O)(=O)c1cccc(c1)C(=O)N(Cc1cnc3c(n1)C(=O)N(C(C)C)[C@@H]3C)[C@H](CC(C)(C)C)CO2] | 4.13 | 697.305 | 698.3 | J |
| Cmpd | | LCMS Rt | Calc. | | LCMS |
| No. | Structure | (min) | Mass | M + 1 | Method |
| IV-2 | 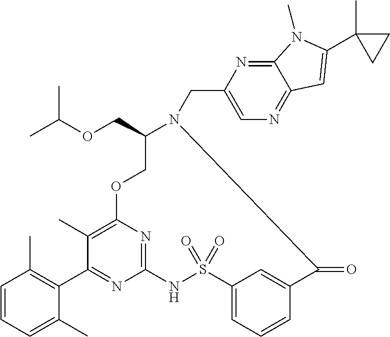 [SMILES: Cc1cccc(C)c1-c1nc2nc(c1C)OC[C@@H](COC(C)C)N(Cc1cnc3cc(C4(C)CC4)n(C)c3n1)C(=O)c1cccc(c1)S(=O)(=O)N2] | 1.73 | 709.305 | 710.3 | A |
| IV-3 | 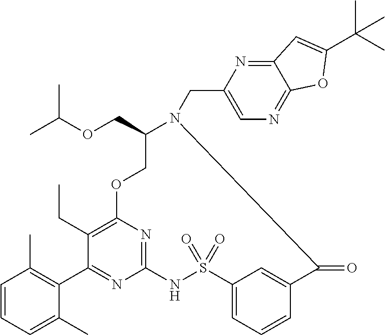 [SMILES: CCc1c2nc(nc1-c1c(C)cccc1C)NS(=O)(=O)c1cccc(c1)C(=O)N(Cc1cnc3oc(C(C)(C)C)cc3n1)[C@H](COC(C)C)CO2] | 2.07 | 712.304 | 713.3 | A |
| IV-5 | 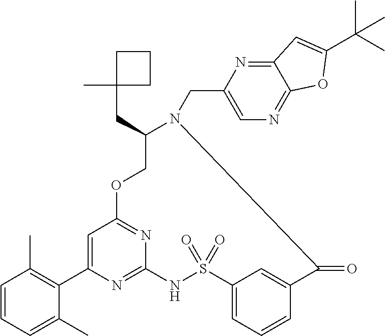 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1(C)CCC1)CO2] | 2.17 | 694.294 | 695.7 | A |
| IV-6 | 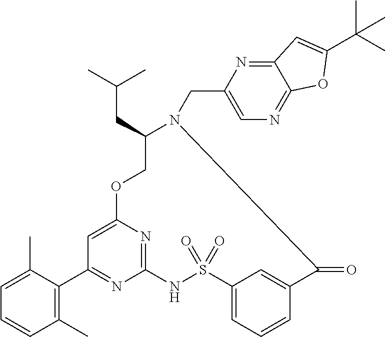 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC(C)C)CO2] | 2.09 | 668.278 | 669.9 | A |
| IV-7 | 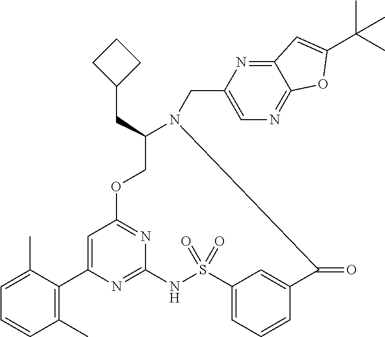 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCC1)CO2] | 2.17 | 680.278 | 681.7 | A |
| IV-8 | 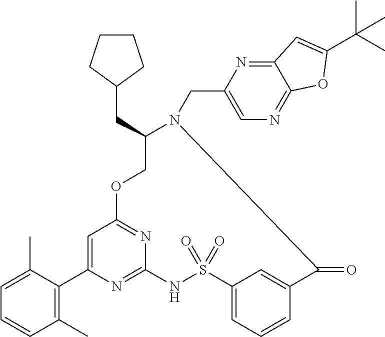 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCCC1)CO2] | 2.19 | 694.294 | 695.7 | A |
| IV-9 | 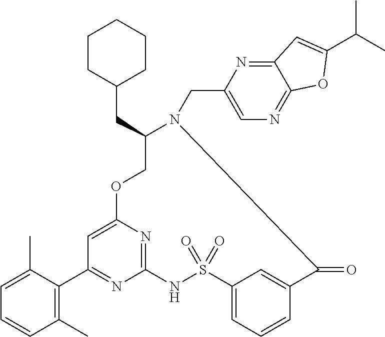 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCCC1)CO2] | 2.26 | 708.309 | 709.7 | A |
| IV-10 | 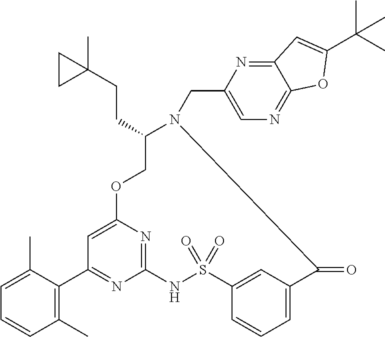 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CCC1(C)CC1)CO2] | 1.97 | 694.294 | 695.9 | A |
| IV-11 | 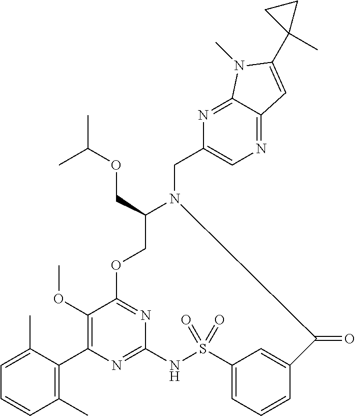 [SMILES: COc1c2nc(nc1-c1c(C)cccc1C)NS(=O)(=O)c1cccc(c1)C(=O)N(Cc1cnc3cc(C4(C)CC4)n(C)c3n1)[C@H](COC(C)C)CO2] | 1.78 | 725.3 | 726.7 | A |
| IV-12 | 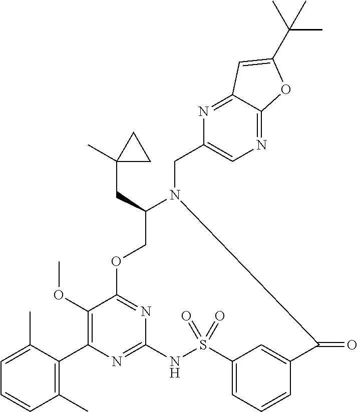 [SMILES: COc1c2nc(nc1-c1c(C)cccc1C)NS(=O)(=O)c1cccc(c1)C(=O)N(Cc1cnc3oc(C(C)(C)C)cc3n1)[C@H](CC1(C)CC1)CO2] | 2.11 | 710.289 | 711.9 | A |
| IV-18 | 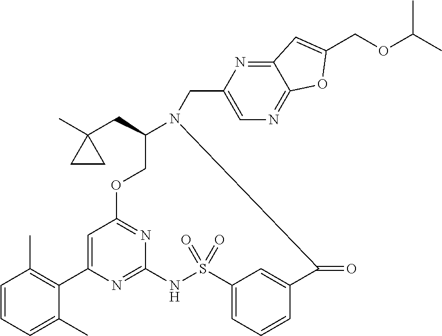 [SMILES: Cc1cccc(C)c1-c1cc2nc(n1)NS(=O)(=O)c1cccc(c1)C(=O)N(Cc1cnc3oc(COC(C)C)cc3n1)[C@H](CC1(C)CC1)CO2] | 1.72 | 696.273 | 697.5 | A |
| IV-19 | 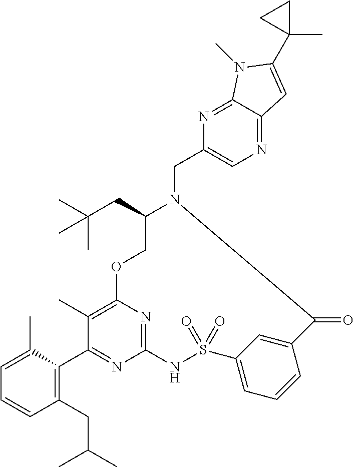 | 2.02 | 749.372 | 750.2 | A |
| IV-25 | 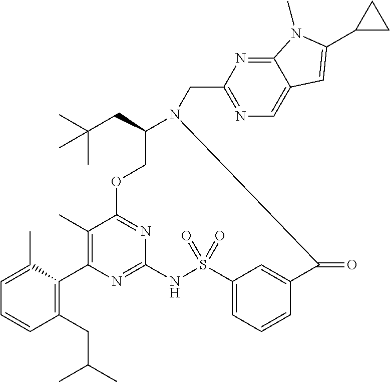 | 1.51 | 735.357 | 736.6 | A |
| IV-27 | 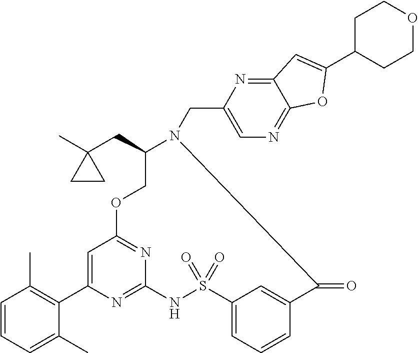 [SMILES: Cc1cccc(C)c1-c1cc2nc(n1)NS(=O)(=O)c1cccc(c1)C(=O)N(Cc1cnc3oc(C4CCOCC4)cc3n1)[C@H](CC1(C)CC1)CO2] | 1.63 | 708.273 | 709.5 | A |
| IV-28 | 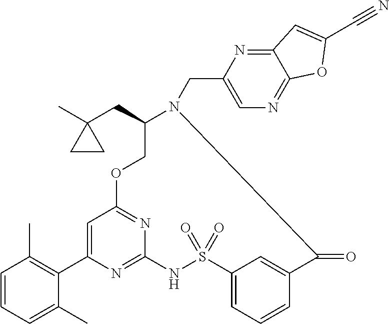 [SMILES: Cc1cccc(C)c1-c1cc2nc(n1)NS(=O)(=O)c1cccc(c1)C(=O)N(Cc1cnc3oc(C#N)cc3n1)[C@H](CC1(C)CC1)CO2] | 1.58 | 649.211 | 650.5 | A |
| IV-29 | 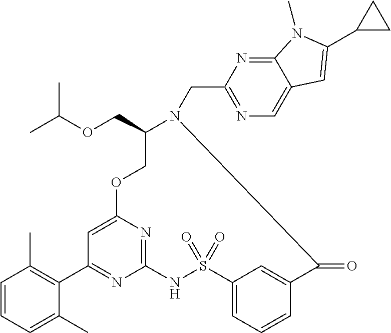 [SMILES: Cc1cccc(C)c1-c1cc2nc(n1)NS(=O)(=O)c1cccc(c1)C(=O)N(Cc1ncc3cc(C4CC4)n(C)c3n1)[C@H](COC(C)C)CO2] | 1.22 | 681.273 | 682.6 | A |
| IV-30 | 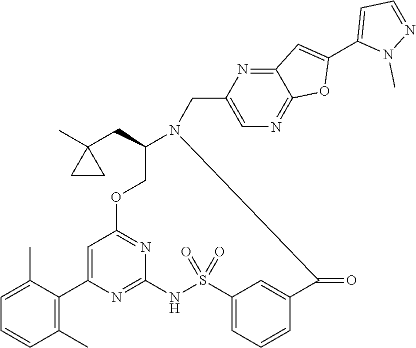 [SMILES: Cc1cccc(C)c1-c1cc2nc(n1)NS(=O)(=O)c1cccc(c1)C(=O)N(Cc1cnc3oc(-c4ccnn4C)cc3n1)[C@H](CC1(C)CC1)CO2] | 1.59 | 704.253 | 705.6 | A |
| IV-231 | 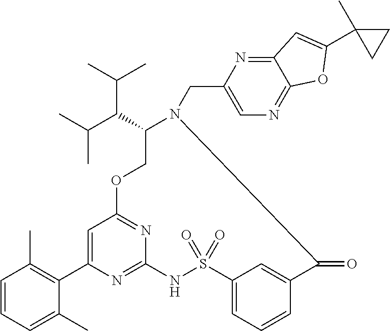 [SMILES: Cc1cccc(C)c1-c1cc2nc(n1)NS(=O)(=O)c1cccc(c1)C(=O)N(Cc1cnc3oc(C4(C)CC4)cc3n1)[C@@H](C(C(C)C)C(C)C)CO2] | 2.2 | 708.309 | 709.8 | A |
| IV-32 | 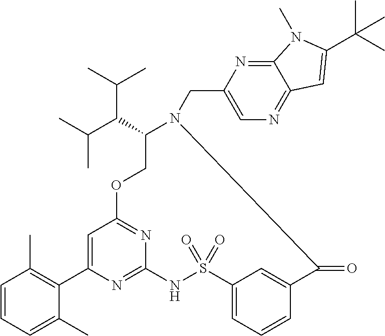 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](C(C(C)C)C(C)C)CO2] | 1.99 | 723.357 | 724.8 | A |
| IV-33 | 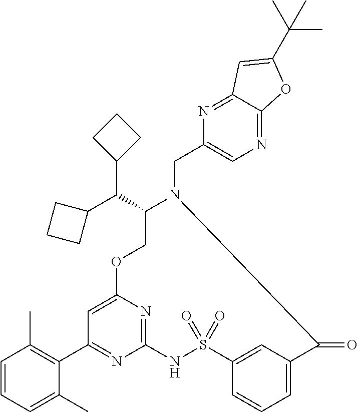 [SMILES: Cc1cccc(C)c1-c1cc2nc(n1)NS(=O)(=O)c1cccc(c1)C(=O)N(Cc1cnc3oc(C(C)(C)C)cc3n1)[C@@H](C(C1CCC1)C1CCC1)CO2] | 2.29 | 734.325 | 735.6 | A |
| IV-34 | 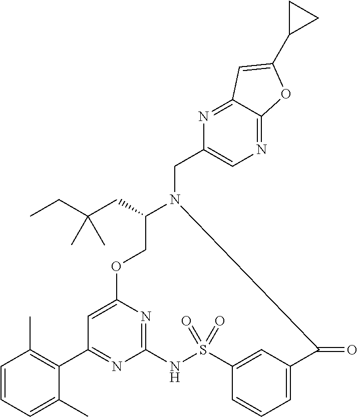 [SMILES: CCC(C)(C)C[C@H]1COc2cc(-c3c(C)cccc3C)nc(n2)NS(=O)(=O)c2cccc(c2)C(=O)N1Cc1cnc2oc(C3CC3)cc2n1] | 1.96 | 680.278 | 681.1 | A |
| IV-36 | 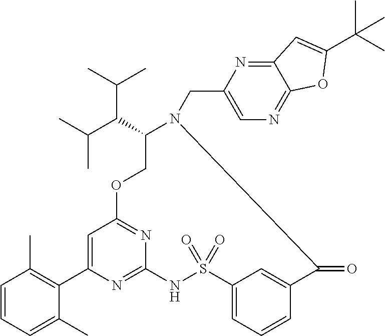 [SMILES: Cc1cccc(C)c1-c1cc2nc(n1)NS(=O)(=O)c1cccc(c1)C(=O)N(Cc1cnc3oc(C(C)(C)C)cc3n1)[C@@H](C(C(C)C)C(C)C)CO2] | 2.25 | 710.325 | 711.8 | A |
| IV-37 | 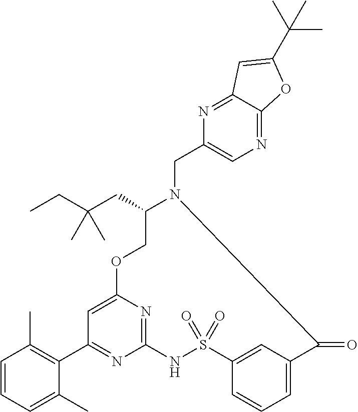 [SMILES: CCC(C)(C)C[C@H]1COc2cc(-c3c(C)cccc3C)nc(n2)NS(=O)(=O)c2cccc(c2)C(=O)N1Cc1cnc2oc(C(C)(C)C)cc2n1] | 2.1 | 696.309 | 697.2 | A |
| IV-38 | 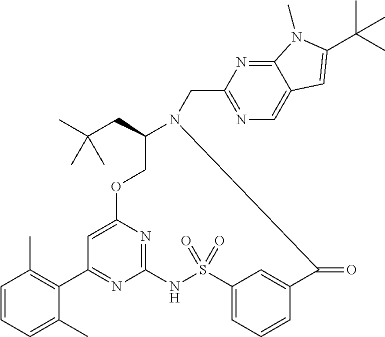 [SMILES: Cc1cccc(C)c1-c1cc2nc(n1)NS(=O)(=O)c1cccc(c1)C(=O)N(Cc1ncc3cc(C(C)(C)C)n(C)c3n1)[C@H](CC(C)(C)C)CO2] | 1.5 | 695.325 | 696.2 | A |
| IV-39 | 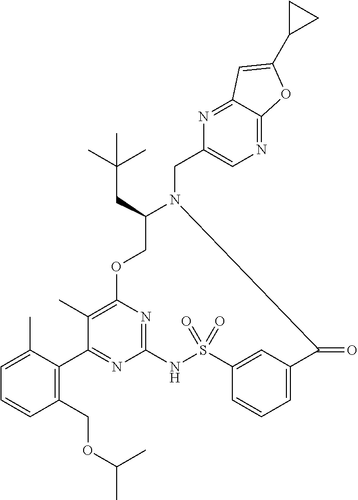 [SMILES: Cc1cccc(COC(C)C)c1-c1nc2nc(c1C)OC[C@@H](CC(C)(C)C)N(Cc1cnc3oc(C4CC4)cc3n1)C(=O)c1cccc(c1)S(=O)(=O)N2] | 2.08 | 738.32 | 739.9 | A |
| IV-41 | 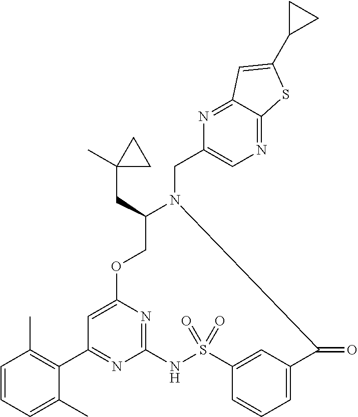 [SMILES: Cc1cccc(C)c1-c1cc2nc(n1)NS(=O)(=O)c1cccc(c1)C(=O)N(Cc1cnc3sc(C4CC4)cc3n1)[C@H](CC1(C)CC1)CO2] | 1.97 | 680.224 | 681.6 | A |
| IV-43 | 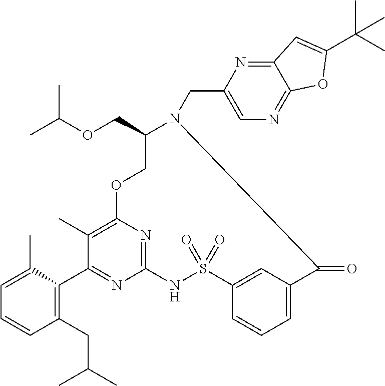 | 2.1 | 740.336 | 741.6 | A |
| IV-44 | 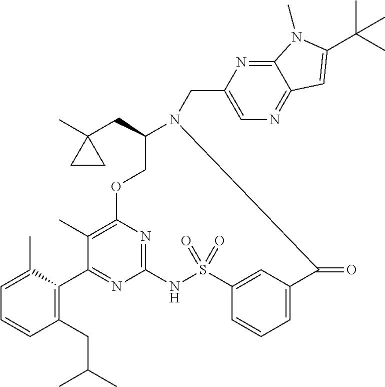 | 1.88 | 749.372 | 750.6 | A |
| IV-45 | 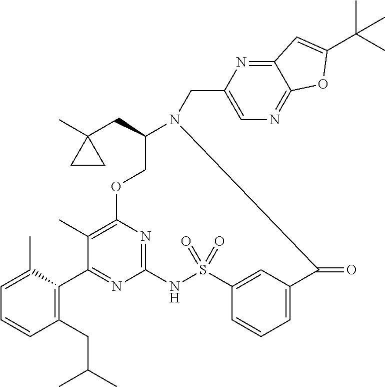 | 2.12 | 736.341 | 737.7 | A |
| IV-46 | 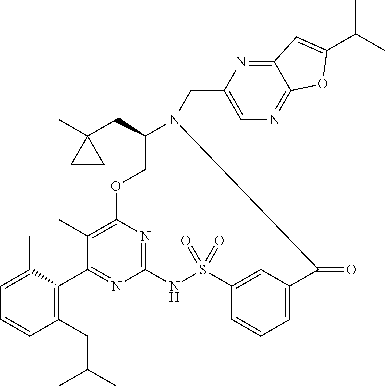 | 2.06 | 722.325 | 723.6 | A |
| IV-47 | 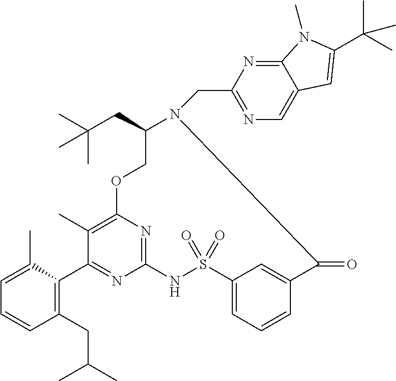 | 1.73 | 751.388 | 752.2 | A |
| IV-50 | 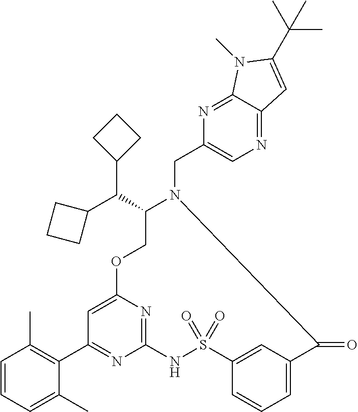 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](C(C1CCC1)C1CCC1)CO2] | 2.03 | 747.357 | 748.8 | A |
| IV-57 | 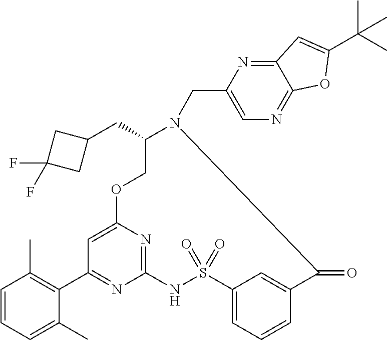 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CC1CC(F)(F)C1)CO2] | 1.79 | 716.259 | 717.5 | A |
| IV-58 | 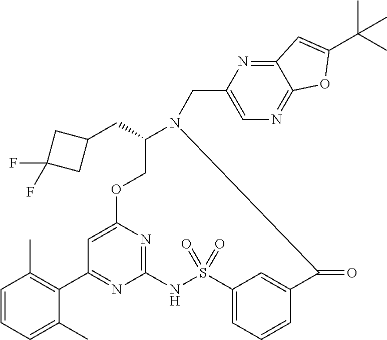 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CC1CC(F)(F)C1)CO2] | 1.79 | 716.259 | 717.6 | A |
| IV-59 | 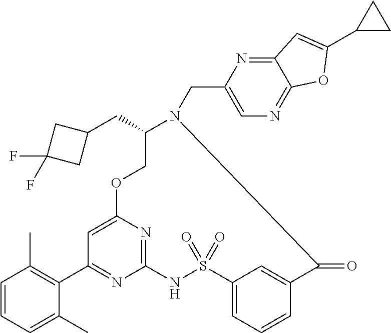 [SMILES: Cc1cccc(C)c1-c1cc2nc(n1)NS(=O)(=O)c1cccc(c1)C(=O)N(Cc1cnc3oc(C4CC4)cc3n1)[C@@H](CC1CC(F)(F)C1)CO2] | 1.64 | 700.228 | 701.5 | A |
| IV-62 | 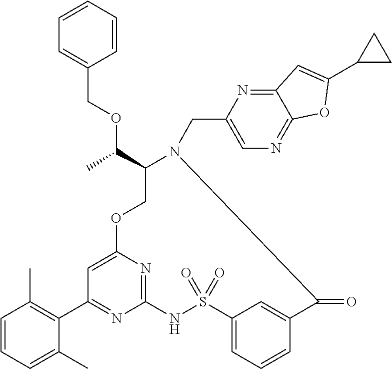 [SMILES: Cc1cccc(C)c1-c1cc2nc(n1)NS(=O)(=O)c1cccc(c1)C(=O)N(Cc1cnc3oc(C4CC4)cc3n1)[C@H]([C@H](C)OCc1ccccc1)CO2] | 2.04 | 730.257 | 731.7 | A |
| IV-64 | 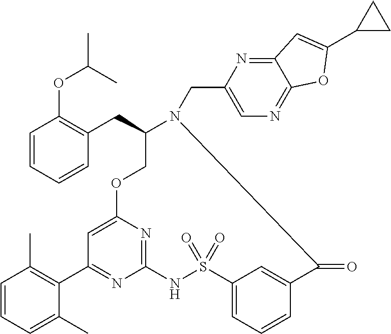 [SMILES: Cc1cccc(C)c1-c1cc2nc(n1)NS(=O)(=O)c1cccc(c1)C(=O)N(Cc1cnc3oc(C4CC4)cc3n1)[C@H](Cc1ccccc1OC(C)C)CO2] | 1.81 | 744.273 | 745.4 | A |
| IV-65 | 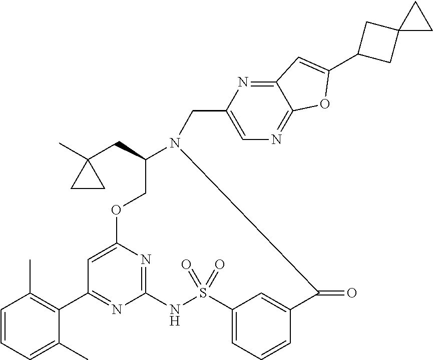 [SMILES: Cc1cccc(C)c1-c1cc2nc(n1)NS(=O)(=O)c1cccc(c1)C(=O)N(Cc1cnc3oc(C4CC5(CC5)C4)cc3n1)[C@H](CC1(C)CC1)CO2] | 2.04 | 704.278 | 705.2 | A |
| IV-66 | 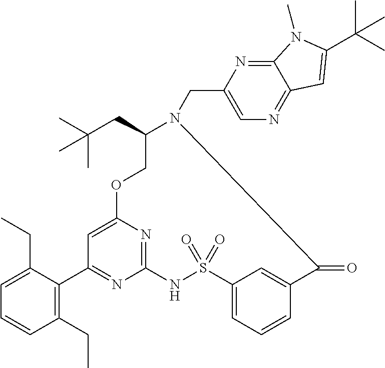 [SMILES: CCc1cccc(CC)c1-c1cc2nc(n1)NS(=O)(=O)c1cccc(c1)C(=O)N(Cc1cnc3cc(C(C)(C)C)n(C)c3n1)[C@H](CC(C)(C)C)CO2] | 2.09 | 723.357 | 724.4 | A |
| IV-67 | 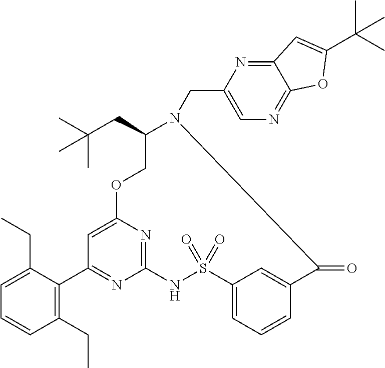 [SMILES: CCc1cccc(CC)c1-c1cc2nc(n1)NS(=O)(=O)c1cccc(c1)C(=O)N(Cc1cnc3oc(C(C)(C)C)cc3n1)[C@H](CC(C)(C)C)CO2] | 2.42 | 710.325 | 711.4 | A |
| IV-68 | 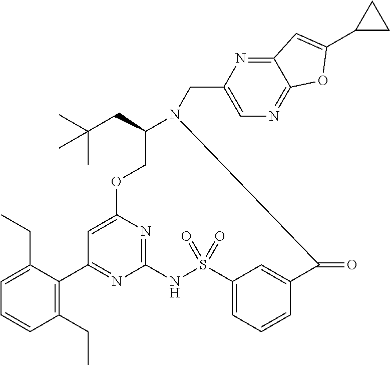 [SMILES: CCc1cccc(CC)c1-c1cc2nc(n1)NS(=O)(=O)c1cccc(c1)C(=O)N(Cc1cnc3oc(C4CC4)cc3n1)[C@H](CC(C)(C)C)CO2] | 2.26 | 694.294 | 695.2 | A |
| IV-69 | 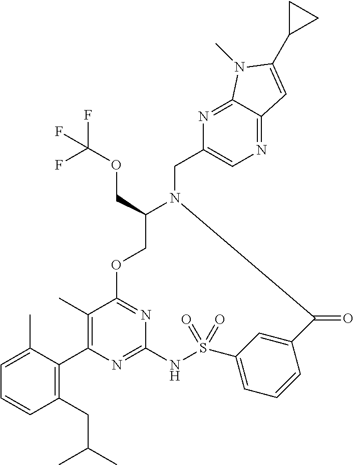 [SMILES: Cc1cccc(CC(C)C)c1-c1nc2nc(c1C)OC[C@@H](COC(F)(F)F)N(Cc1cnc3cc(C4CC4)n(C)c3n1)C(=O)c1cccc(c1)S(=O)(=O)N2] | 1.93 | 763.276 | 764.4 | A |
| IV-71 | 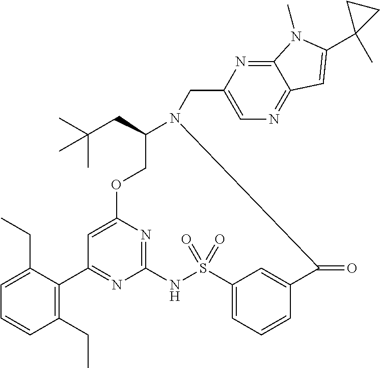 [SMILES: CCc1cccc(CC)c1-c1cc2nc(n1)NS(=O)(=O)c1cccc(c1)C(=O)N(Cc1cnc3cc(C4(C)CC4)n(C)c3n1)[C@H](CC(C)(C)C)CO2] | 1.93 | 721.341 | 722.6 | A |
| IV-72 | 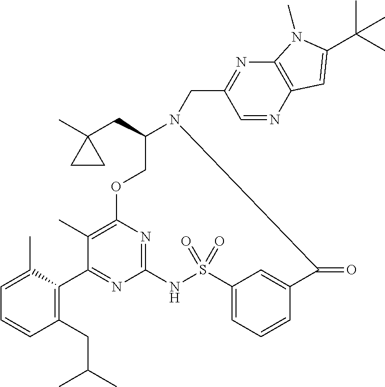 | 1.93 | 750.368 | 751.3 | A |
| IV-73 | 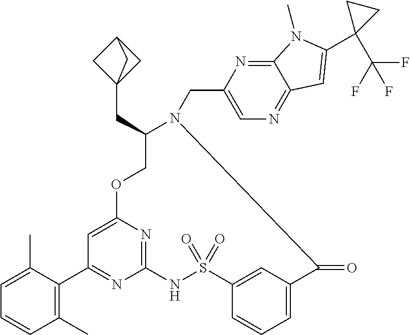 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](CC13CC(C1)C3)CO2] | 2.07 | 757.266 | 758.7 | A |
| IV-74 | 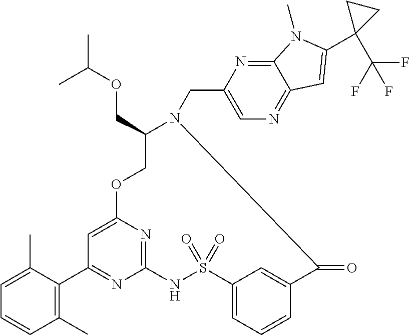 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](COC(C)C)CO2] | 1.96 | 749.261 | 750.7 | A |
| IV-75 | 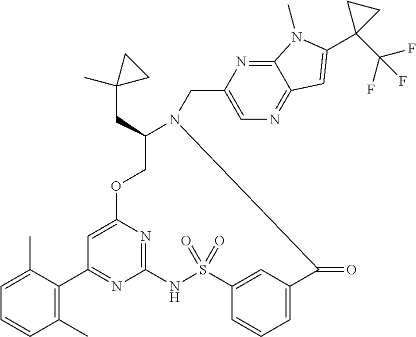 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](CC1(C)CC1)CO2] | 1.99 | 745.266 | 746.7 | A |
| IV-77 | 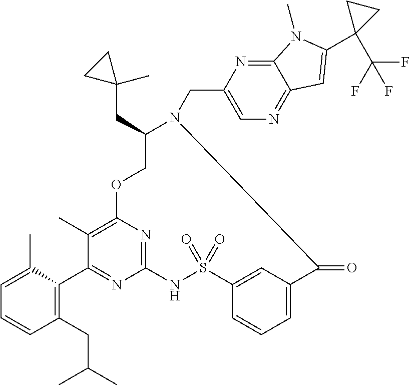 | 2.28 | 801.328 | 802.8 | A |
| IV-78 | 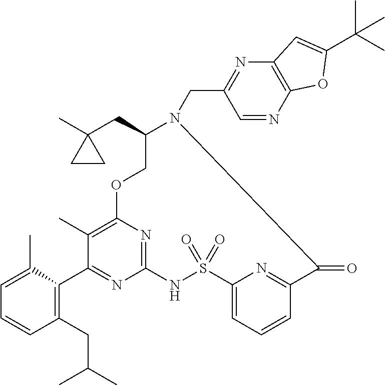 | 2.14 | 737.336 | 738.5 | A |
| IV-79 | 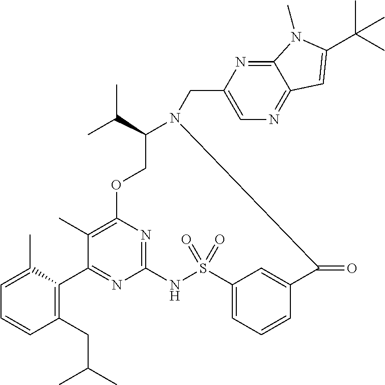 | 1.93 | 723.357 | 724.6 | A |
| IV-80 | 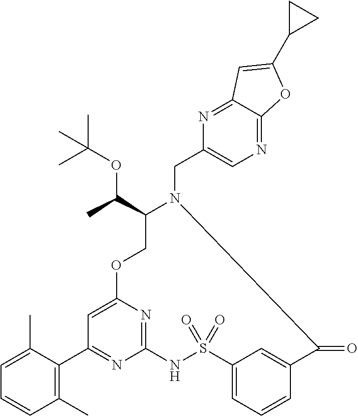 [SMILES: Cc1cccc(C)c1-c1cc2nc(n1)NS(=O)(=O)c1cccc(c1)C(=O)N(Cc1cnc3oc(C4CC4)cc3n1)[C@H]([C@@H](C)OC(C)(C)C)CO2] | 1.96 | 696.273 | 697.7 | A |
| IV-81 | 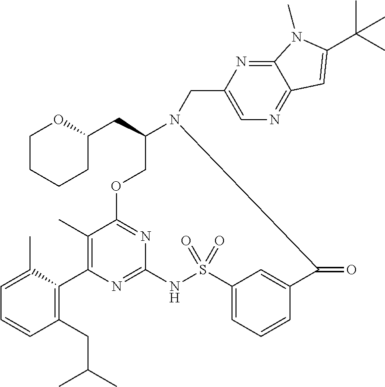 | 1.96 | 779.383 | 780.8 | A |
| IV-83 | 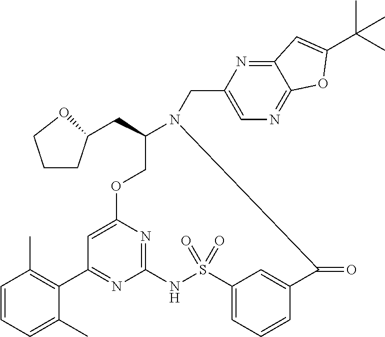 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@@H]1CCCO1)CO2] | 1.98 | 696.273 | 697.95 | A |
| IV-84 | 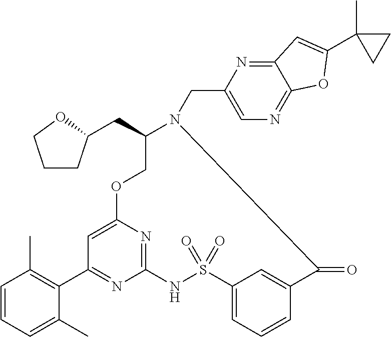 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@@H]1CCCO1)CO2] | 1.92 | 694.257 | 695.97 | A |
| IV-85 | 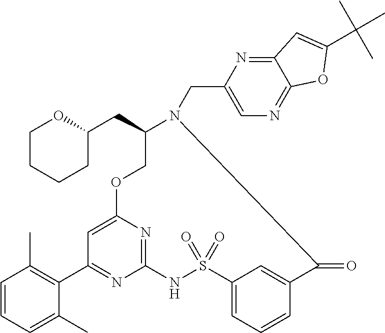 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@@H]1CCCCO1)CO2] | 1.94 | 710.289 | 711.5 | A |
| IV-86 | 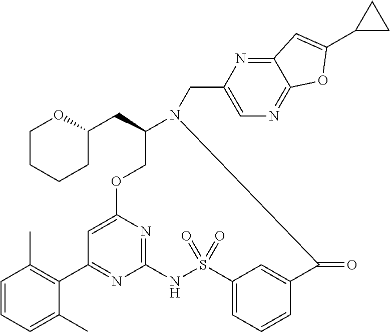 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCCO1)CO2] | 1.78 | 694.257 | 695.4 | A |
| IV-87 | 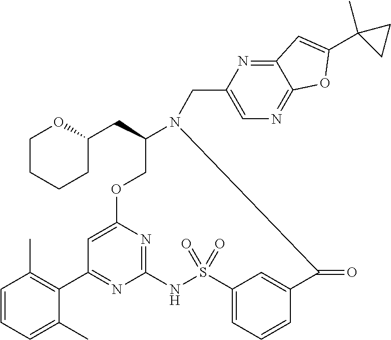 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@@H]1CCCCO1)CO2] | 1.88 | 708.273 | 709.5 | A |
| IV-88 | 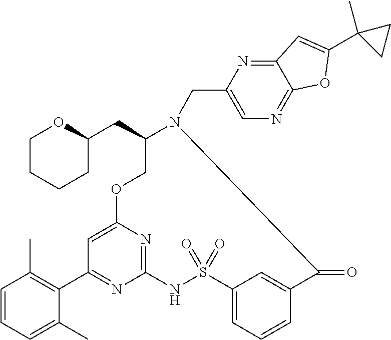 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@H]1CCCCO1)CO2] | 1.86 | 708.273 | 709.5 | A |
| IV-90 | 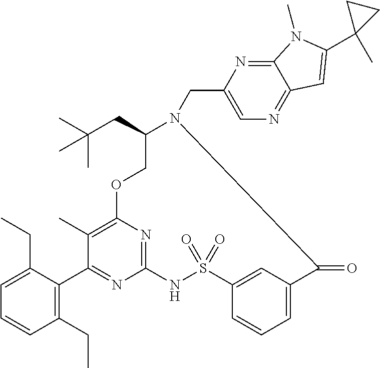 [SMILES: CCc1cccc(CC)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] | 1.95 | 735.357 | 736.6 | A |
| IV-90 | 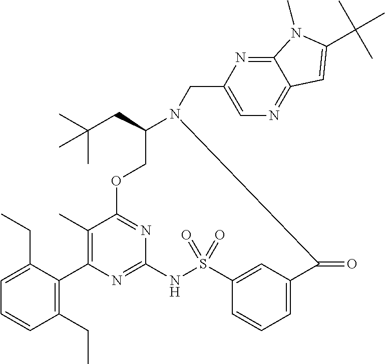 [SMILES: CCc1cccc(CC)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2] | 1.96 | 737.372 | 738.6 | A |
| IV-94 | 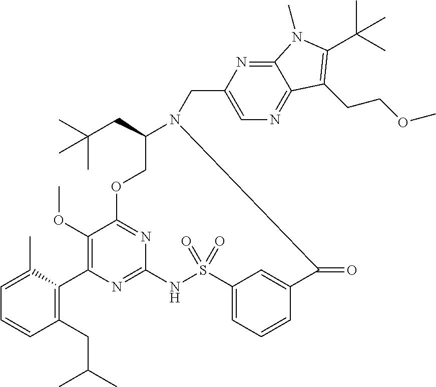 | 2.37 | 825.425 | 826.4 | A |
| IV-95 | 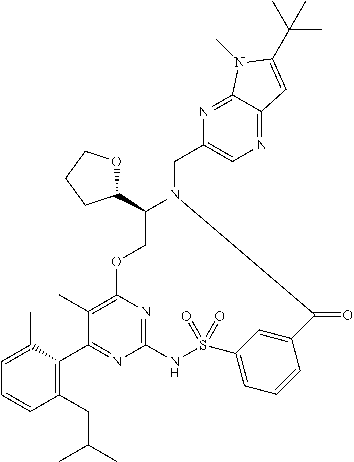 | 1.95 | 751.352 | 752.8 | A |
| IV-99 | 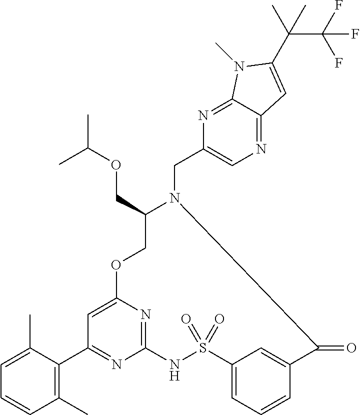 [SMILES: Cc1cccc(C)c1-c1cc2nc(n1)NS(=O)(=O)c1cccc(c1)C(=O)N(Cc1cnc3cc(C(C)(C)C(F)(F)F)n(C)c3n1)[C@H](COC(C)C)CO2] | 1.95 | 751.276 | 752.7 | A |
| IV-100 | 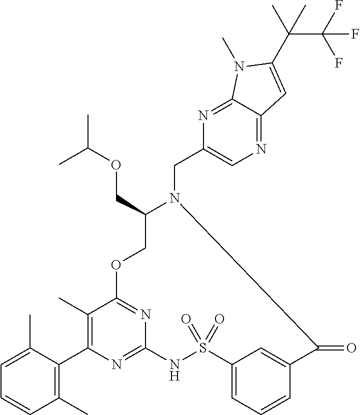 [SMILES: Cc1cccc(C)c1-c1nc2nc(c1C)OC[C@@H](COC(C)C)N(Cc1cnc3cc(C(C)(C)C(F)(F)F)n(C)c3n1)C(=O)c1cccc(c1)S(=O)(=O)N2] | 2.01 | 765.292 | 766.7 | A |
| IV-101 | 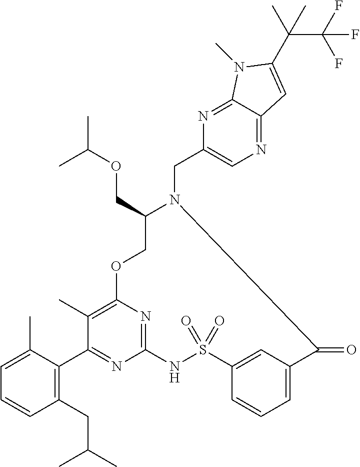 [SMILES: Cc1cccc(CC(C)C)c1-c1nc2nc(c1C)OC[C@@H](COC(C)C)N(Cc1cnc3cc(C(C)(C)C(F)(F)F)n(C)c3n1)C(=O)c1cccc(c1)S(=O)(=O)N2] | 2.23 | 807.339 | 808.8 | A |
| IV-103 | 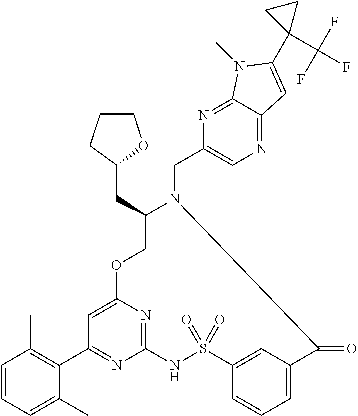 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](C[C@@H]1CCCO1)CO2] | 1.83 | 761.261 | 762.7 | A |
| IV-104 | 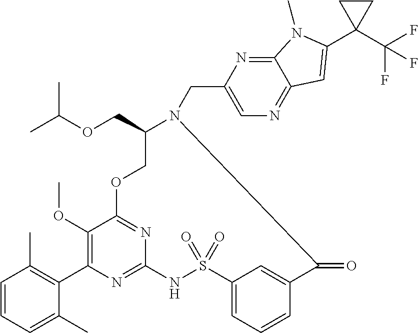 [SMILES: COc1c2nc(nc1-c1c(C)cccc1C)NS(=O)(=O)c1cccc(c1)C(=O)N(Cc1cnc3cc(C4(C(F)(F)F)CC4)n(C)c3n1)[C@H](COC(C)C)CO2] | 1.99 | 779.271 | 780.2 | A |
| IV-105 | 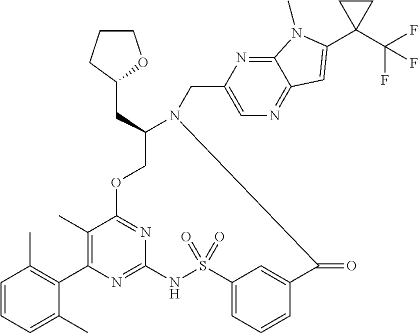 [SMILES: Cc1cccc(C)c1-c1nc2nc(c1C)OC[C@@H](C[C@@H]1CCCO1)N(Cc1cnc3cc(C4(C(F)(F)F)CC4)n(C)c3n1)C(=O)c1cccc(c1)S(=O)(=O)N2] | 1.9 | 775.276 | 776.2 | A |
| IV-106 | 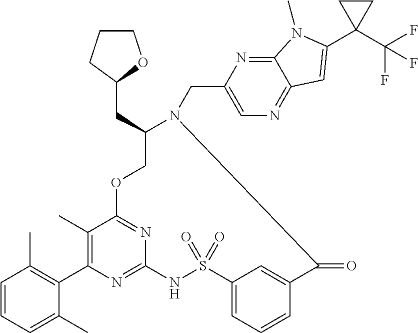 [SMILES: Cc1cccc(C)c1-c1nc2nc(c1C)OC[C@@H](C[C@H]1CCCO1)N(Cc1cnc3cc(C4(C(F)(F)F)CC4)n(C)c3n1)C(=O)c1cccc(c1)S(=O)(=O)N2] | 1.88 | 775.276 | 776.3 | A |

| Compound | |
|---|---|
| Number | NMR |
| I-1 | ¹H NMR (400 MHz, CDCl₃) δ 8.67 (t, J = 1.8 Hz, 1H), 8.41 (s, 1H), 8.10 (d, J = |
| | 7.9 Hz, 1H), 7.81 (dt, J = 7.6, 1.4 Hz, 1H), 7.62 (t, J = 7.8 Hz, 1H), 7.19 (t, |
| | J = 7.6 Hz, 1H), 7.00 (d, J = 7.6 Hz, 2H), 6.62 (d, J = 1.0 Hz, 1H), 6.15 (s, |
| | 1H), 5.41 (dd, J = 11.1, 4.1 Hz, 1H), 5.32 (d, J = 14.9 Hz, 1H), 4.30 (d, J = |
| | 14.9 Hz, 1H), 4.25 (t, J = 11.3 Hz, 1H), 4.10 (tt, J = 11.2, 3.7 Hz, 1H), 3.22- |
| | 3.10 (m, 1H), 1.95 (s, 6H), 1.81 (ddd, J = 14.1, 10.7, 3.1 Hz, 1H), 1.55-1.44 |
| | (m, 1H), 1.40 (d, J = 6.9 Hz, 6H), 1.39-1.33 (m, 1H), 0.85 (d, J = 6.6 Hz, |
| | 3H), 0.46 (d, J = 6.4 Hz, 3H). |
| I-2 | ¹H NMR (400 MHz, CDCl₃) δ 8.61 (t, J = 1.9 Hz, 1H), 8.43 (s, 1H), 8.11 (d, J = |
| | 8.0 Hz, 1H), 7.80 (dt, J = 7.7, 1.3 Hz, 1H), 7.65 (t, J = 7.8 Hz, 1H), 7.19 (t, |
| | J = 7.6 Hz, 1H), 6.99 (d, J = 7.6 Hz, 2H), 6.61 (d, J = 1.0 Hz, 1H), 6.13 (s, |
| | 1H), 5.41-5.29 (m, 2H), 4.33-4.20 (m, 2H), 4.04 (tt, J = 11.3, 3.8 Hz, 1H), |
| | 3.22-3.10 (m, 1H), 1.94 (s, 6H), 1.81-1.70 (m, 1H), 1.68-1.54 (m, 1H), |
| | 1.40 (d, J = 6.9 Hz, 6H), 1.38-1.30 (m, 1H), 1.18-1.06 (m, 1H), 0.97-0.87 |
| | (m, 1H), 0.82 (d, J = 6.5 Hz, 3H), 0.80 (d, J = 6.5 Hz, 3H). |
| I-3 | ¹H NMR (400 MHz, Chloroform-d) δ 8.86 (s, 1H), 8.51 (s, 1H), 8.05 (d, J = |
| | 8.0 Hz, 1H), 7.87-7.81 (m, 1H), 7.66 (t, J = 7.7 Hz, 1H), 7.22 (t, J = 7.6 Hz, |
| | 1H), 7.07 (d, J = 7.6 Hz, 2H), 6.76 (s, 1H), 6.24 (s, 1H), 5.52 (dd, J = 11.2, |
| | 4.1 Hz, 1H), 5.40 (d, J = 15.8 Hz, 1H), 4.39 (d, J = 15.9 Hz, 1H), 4.34-4.27 |
| | (m, 1H), 4.13 (t, J = 11.4 Hz, 1H), 3.97 (s, 3H), 3.94-3.85 (m, 1H), 3.28- |
| | 3.14 (m, 2H), 3.08-2.97 (m, 1H), 2.05 (s, 6H), 1.98-1.87 (m, 1H), 1.79- |
| | 1.71 (m, 2H), 1.55-1.48 (m, 1H), 1.48-1.45 (m, 1H), 1.44-1.38 (m, 6H), |
| | 1.38-1.31 (m, 1H), 1.27 (d, J = 13.9 Hz, 1H), 1.19-1.05 (m, 1H). |
| I-4 | ¹H NMR (400 MHz, Chloroform-d) δ 8.87 (t, J = 1.8 Hz, 1H), 8.33 (s, 1H), |
| | 8.05 (dt, J = 8.0, 1.4 Hz, 1H), 7.85 (dt, J = 7.8, 1.3 Hz, 1H), 7.64 (t, J = 7.8 |
| | Hz, 1H), 7.21 (t, J = 7.6 Hz, 1H), 7.07 (d, J = 7.6 Hz, 2H), 6.27 (s, 1H), 5.58- |
| | 5.47 (m, 1H), 5.28 (d, J = 15.1 Hz, 1H), 4.37 (d, J = 15.2 Hz, 1H), 4.28-4.11 |
| | (m, 2H), 3.27 (hept, J = 7.0 Hz, 1H), 2.31 (s, 3H), 2.02 (s, 6H), 1.80 (dd, J = |
| | 15.2, 7.0 Hz, 1H), 1.61 (d, J = 14.9 Hz, 1H), 1.37 (dd, J = 6.9, 5.9 Hz, 6H), |
| | 0.64 (s, 9H). |
| I-5 | ¹H NMR (400 MHz, CDCl₃) δ 8.43 (s, 1H), 8.42 (t, J = 1.7 Hz, 1H), 8.07 (s, |
| | 1H), 7.95 (dt, J = 8.0, 1.6 Hz, 1H), 7.78 (d, J = 7.6 Hz, 1H), 7.59 (t, J = 7.8 |
| | Hz, 1H), 7.20 (t, J = 7.6 Hz, 1H), 7.07 (d, J = 7.6 Hz, 2H), 6.62 (d, J = 1.1 |
| | Hz, 1H), 6.21 (s, 1H), 6.05-5.92 (m, 1H), 4.91 (d, J = 14.9 Hz, 1H), 4.60 (d, |
| | J = 14.8 Hz, 1H), 4.27-4.11 (m, 2H), 3.24-3.09 (m, 1H), 2.71 (d, J = 14.8 |
| | Hz, 1H), 2.21-2.09 (m, 1H), 2.01 (s, 6H), 1.76-1.66 (m, 1H), 1.41 (d, J = |
| | 6.9 Hz, 6H), 1.29-1.19 (m, 1H), 1.18-1.07 (m, 1H), 0.59 (d, J = 6.5 Hz, |
| | 3H), −0.02 (d, J = 6.0 Hz, 3H). |
| I-6 | ¹H NMR (400 MHz, CDCl₃) δ 9.25 (s, 1H), 8.12 (d, J = 8.0 Hz, 1H), 7.94- |
| | 7.89 (m, 1H), 7.68 (t, J = 7.7 Hz, 1H), 7.48 (d, J = 10.4 Hz, 1H), 7.20 (t, J = |
| | 7.6 Hz, 1H), 7.07 (d, J = 7.6 Hz, 2H), 6.23 (s, 1H), 6.15 (s, 1H), 5.65 (dd, J = |
| | 11.4, 4.2 Hz, 1H), 5.56-5.49 (m, 1H), 4.40 (d, J = 16.8 Hz, 1H), 4.30-4.22 |
| | (m, 1H), 3.89 (s, 3H), 3.81 (t, J = 11.4 Hz, 1H), 3.12-3.04 (m, 1H), 2.02 (s, |
| | 6H), 1.80-1.74 (m, 1H), 1.36-1.28 (m, 7H), 0.67 (s, 9H). |
| I-7 | ¹H NMR (400 MHz, Chloroform-d) δ 8.64 (s, 1H), 8.42 (s, 1H), 8.17 (d, J = |
| | 7.9, 1.5 Hz, 1H), 7.80 (d, J = 7.7, 1.3 Hz, 1H), 7.66 (t, J = 7.8 Hz, 1H), 7.19 |
| | (t, J = 7.6 Hz, 1H), 7.00 (d, J = 7.6 Hz, 2H), 6.61 (d, J = 1.0 Hz, 1H), 6.14 (s, |
| | 1H), 5.41-5.28 (m, 2H), 4.36-4.21 (m, 3H), 3.94-3.87 (m, 1H), 3.27-3.11 |
| | (m, 2H), 3.06-2.98 (m, 1H), 1.96 (s, 6H), 1.77-1.66 (m, 3H), 1.49-1.43 (m, |
| | 2H), 1.40 (d, J = 6.9 Hz, 6H), 1.38-1.32 (m, 1H), 1.29-1.22 (m, 1H), 1.15- |
| | 1.04 (m, 1H). |
| I-8 | ¹H NMR (400 MHz, Chloroform-d) δ 8.60 (s, 1H), 8.39 (s, 1H), 7.74 (d, J = |
| | 7.5 Hz, 1H), 7.45 (t, J = 7.7 Hz, 1H), 7.38-7.29 (m, 1H), 7.26 (t, J = 7.6 Hz, |
| | 1H), 7.08 (d, J = 7.6 Hz, 2H), 6.63 (s, 1H), 6.18 (s, 1H), 5.50-5.39 (m, 2H), |
| | 4.39-4.29 (m, 1H), 4.29-4.16 (m, 2H), 3.87-3.77 (m, 1H), 3.41 (t, J = 11.3 |
| | Hz, 1H), 3.26-3.10 (m, 2H), 2.10 (s, 6H), 1.89-1.75 (m, 3H), 1.60-1.46 (m, |
| | 4H), 1.40 (d, J = 6.8 Hz, 6H), 1.26-1.15 (m, 1H). |
| I-9 | ¹H NMR (400 MHz, Chloroform-d) δ 8.66 (t, J = 1.8 Hz, 1H), 8.41 (s, 1H), |
| | 8.12 (d, J = 8.1, 1.4 Hz, 1H), 7.77 (d, J = 7.7, 1.4 Hz, 1H), 7.62 (t, J = 7.8 Hz, |
| | 1H), 7.19 (t, J = 7.6 Hz, 1H), 7.00 (d, J = 7.6 Hz, 2H), 6.62 (d, J = 1.0 Hz, |
| | 1H), 6.14 (s, 1H), 5.39 (dd, J = 10.8, 3.7 Hz, 1H), 5.31 (d, J = 14.9 Hz, 1H), |
| | 4.31 (d, J = 14.9 Hz, 1H), 4.24 (t, J = 11.2 Hz, 1H), 4.19-4.10 (m, 1H), 3.16 |
| | (hept, J = 6.8, 1.1 Hz, 1H), 1.95 (s, 6H), 1.77-1.71 (m, 1H), 1.71-1.56 (m, |
| | 3H), 1.56-1.47 (m, 2H), 1.40 (d, J = 6.9 Hz, 6H), 1.25-1.06 (m, 3H), 1.06- |
| | 0.97 (m, 2H), 0.95-0.84 (m, 1H), 0.47-0.36 (m, 1H). |
| I-10 | ¹H NMR (400 MHz, Chloroform-d) δ 9.67 (s, 1H), 8.73 (t, J = 1.8 Hz, 1H), |
| | 8.32 (s, 1H), 7.82 (dt, J = 7.6, 1.4 Hz, 1H), 7.66 (d, J = 8.0 Hz, 1H), 7.56 (t, |
| | J = 7.7 Hz, 1H), 7.23 (d, J = 7.6 Hz, 1H), 7.08 (d, J = 7.6 Hz, 2H), 6.24 (s, 1H), |
| | 5.43 (dd, J = 11.2, 4.3 Hz, 1H), 5.37 (d, J = 15.3 Hz, 1H), 4.61 (d, J = 15.3 |
| | Hz, 1H), 4.45 (t, J = 11.5 Hz, 1H), 4.39-4.28 (m, 1H), 3.70-3.59 (m, 2H), |
| | 3.53-3.44 (m, 1H), 3.32-3.22 (m, 1H), 2.30 (s, 3H), 2.08 (s, 6H), 1.37 (dd, |
| | J = 6.9, 4.5 Hz, 6H), 1.13 (d, J = 6.1 Hz, 3H), 1.08 (d, J = 6.1 Hz, 3H). |
| I-11 | ¹H NMR (400 MHz, Chloroform-d) δ 9.52 (s, 1H), 8.72 (d, J = 1.8 Hz, 1H), |
| | 8.37 (s, 1H), 8.06 (d, J = 7.8 Hz, 1H), 7.84 (dt, J = 7.7, 1.3 Hz, 1H), 7.66 (t, |
| | J = 7.8 Hz, 1H), 7.20 (t, J = 7.6 Hz, 1H), 7.04 (d, J = 7.6 Hz, 2H), 6.22 (s, 1H), |
| | 5.42 (dd, J = 11.4, 4.1 Hz, 1H), 5.36 (d, J = 15.1 Hz, 1H), 4.33-4.22 (m, 2H), |
| | 4.08-3.98 (m, 1H), 3.34-3.20 (m, 1H), 2.31 (s, 3H), 2.02 (s, 6H), 1.73-1.52 |
| | (m, 3H), 1.38 (dd, J = 7.0, 4.6 Hz, 6H), 1.19 (td, J = 12.7, 4.9 Hz, 1H), 0.83 |
| | (s, 9H). |
| I-12 | ¹H NMR (400 MHz, Chloroform-d) δ 9.52 (s, 1H), 8.77 (t, J = 1.8 Hz, 1H), |
| | 8.35 (s, 1H), 8.05 (dd, J = 8.0, 1.7 Hz, 1H), 7.82 (dt, J = 7.8, 1.3 Hz, 1H), |
| | 7.64 (t, J = 7.8 Hz, 1H), 7.25-7.17 (m, 1H), 7.06 (d, J = 7.6 Hz, 2H), 6.24 (s, |
| | 1H), 5.49-5.40 (m, 1H), 5.34 (d, J = 15.2 Hz, 1H), 4.34 (d, J = 15.2 Hz, 1H), |
| | 4.28-4.15 (m, 2H), 3.33-3.22 (m, 1H), 2.31 (s, 3H), 2.03 (s, 6H), 1.77-1.70 |
| | (m, 1H), 1.65-1.52 (m, 3H), 1.50-1.43 (m, 2H), 1.37 (dd, J = 6.9, 4.2 Hz, |
| | 6H), 1.22-1.07 (m, 2H), 1.07-0.97 (m, 2H), 0.97-0.87 (m, 2H), 0.43-0.30 |
| | (m, 1H). |
| I-14 | ¹H NMR (400 MHz, CDCl₃) δ 9.07 (s, 1H), 7.87-7.82 (m, 1H), 7.53 (t, J = |
| | 7.7 Hz, 1H), 7.47 (d, J = 10.3 Hz, 2H), 7.28-7.22 (m, 1H), 7.08 (d, J = 7.6 |
| | Hz, 2H), 6.19 (s, 1H), 6.14 (s, 1H), 5.64-5.57 (m, 1H), 5.52 (dd, J = 11.4, 4.4 |
| | Hz, 1H), 4.66-4.59 (m, 1H), 4.40-4.31 (m, 1H), 4.03 (t, J = 11.6 Hz, 1H), |
| | 3.88 (s, 3H), 3.70-3.60 (m, 2H), 3.56 (p, J = 6.1 Hz, 1H), 3.08 (hept, J = 6.5 |
| | Hz, 1H), 2.07 (s, 6H), 1.32 (dd, J = 13.6, 6.8 Hz, 6H), 1.16 (d, J = 6.1 Hz, |
| | 3H), 1.12 (d, J = 6.1 Hz, 3H). |
| I-16 | ¹H NMR (400 MHz, CDCl₃) δ 8.83 (t, J = 1.8 Hz, 1H), 8.56 (s, 1H), 7.93 (d, J = |
| | 7.9 Hz, 1H), 7.81 (dt, J = 7.6, 1.3 Hz, 1H), 7.62 (t, J = 7.8 Hz, 1H), 7.24 (t, |
| | J = 7.6 Hz, 1H), 7.06 (d, J = 7.7 Hz, 2H), 6.89 (s, 1H), 6.20 (s, 1H), 5.55 (dd, |
| | J = 10.8, 3.9 Hz, 1H), 5.43 (d, J = 15.9 Hz, 1H), 4.48 (d, J = 16.0 Hz, 1H), |
| | 4.35-4.24 (m, 1H), 4.17 (t, J = 11.2 Hz, 1H), 4.01 (s, 3H), 3.81-3.72 (m, |
| | 1H), 3.66-3.54 (m, 2H), 3.29-3.19 (m, 1H), 2.05 (s, 6H), 2.03-1.96 (m, |
| | 1H), 1.88 (dt, J = 14.9, 4.3 Hz, 1H), 1.78 (tt, J = 6.6, 4.0 Hz, 3H), 1.43 (d, J = |
| | 6.7 Hz, 3H), 1.41 (d, J = 6.9 Hz, 3H), 1.27-1.14 (m, 1H). |
| I-17 | ¹H NMR (400 MHz, Chloroform-d) δ 8.83 (t, J = 1.8 Hz, 1H), 8.34 (s, 1H), |
| | 8.20-8.03 (m, 2H), 7.88 (dt, J = 7.7, 1.4 Hz, 1H), 7.64 (t, J = 7.8 Hz, 1H), |
| | 7.24-7.18 (m, 1H), 7.07 (d, J = 7.6 Hz, 2H), 6.27 (s, 1H), 5.47 (dd, J = 11.4, |
| | 4.3 Hz, 1H), 5.30 (d, J = 15.0 Hz, 1H), 4.47-4.38 (m, 1H), 4.27-4.13 (m, |
| | 2H), 3.28 (hept, J = 7.0 Hz, 1H), 2.31 (s, 3H), 2.03 (s, 6H), 1.84 (d, J = 14.7 |
| | Hz, 1H), 1.50-1.43 (m, 1H), 1.38 (d, J = 4.5 Hz, 3H), 1.36 (d, J = 4.5 Hz, |
| | 3H), 0.48 (s, 3H), 0.41-0.32 (m, 1H), 0.30-0.21 (m, 1H), 0.17-0.09 (m, |
| | 1H), 0.05-0.01 (overlapped with TMS, m, 1H) |
| I-19 | ¹H NMR (400 MHz, Chloroform-d) δ 9.10-8.40 (m, 2H), 8.36 (s, 1H), 8.03 |
| | (d, J = 7.9 Hz, 1H), 7.85 (dt, J = 7.7, 1.3 Hz, 1H), 7.65 (t, J = 7.8 Hz, 1H), |
| | 7.21 (t, J = 7.6 Hz, 1H), 7.06 (d, J = 7.6 Hz, 2H), 6.24 (s, 1H), 5.42 (dd, J = |
| | 11.3, 4.1 Hz, 1H), 5.36 (d, J = 15.1 Hz, 1H), 4.33-4.21 (m, 2H), 4.14-4.03 |
| | (m, 1H), 3.28 (hept, J = 6.9 Hz, 1H), 2.30 (s, 3H), 2.04 (s, 6H), 1.84-1.62 (m, |
| | 2H), 1.40-1.35 (m, 6H), 1.35-1.27 (m, 1H), 1.20-1.07 (m, 1H), 0.99-0.86 |
| | (m, 1H), 0.80 (d, J = 6.6 Hz, 3H), 0.77 (d, J = 6.5 Hz, 3H) |
| I-20 | ¹H NMR (400 MHz, Chloroform-d) δ 8.78 (t, J = 1.9 Hz, 1H), 8.34 (s, 1H), |
| | 8.08 (d, J = 8.0 Hz, 1H), 7.85 (dt, J = 7.7, 1.4 Hz, 1H), 7.64 (t, J = 7.8 Hz, |
| | 1H), 7.21 (t, J = 7.6 Hz, 1H), 7.07 (d, J = 7.7 Hz, 2H), 6.62 (s, 1H), 6.26 (s, |
| | 1H), 5.43 (dd, J = 10.3, 3.3 Hz, 1H), 5.23 (d, J = 14.9 Hz, 1H), 4.33 (d, J = |
| | 14.9 Hz, 1H), 4.30-4.12 (m, 2H), 2.17-2.09 (m, 1H), 2.01 (s, 6H), 1.80 (dd, |
| | J = 15.2, 8.2 Hz, 1H), 1.58 (overlapped with water, d, J = 13.8 Hz, 1H), 1.18- |
| | 1.12 (m, 4H), 0.63 (s, 9H) |
| I-21 | ¹H NMR (400 MHz, CDCl₃) δ 8.86 (t, J = 1.9 Hz, 1H), 8.55 (s, 1H), 7.78 (dt, |
| | J = 7.3, 1.6 Hz, 1H), 7.52 (t, J = 7.6 Hz, 1H), 7.48 (d, J = 8.1 Hz, 1H), 7.26- |
| | 7.24 (m, 1H), 7.09 (d, J = 7.7 Hz, 2H), 6.72 (s, 1H), 6.18 (s, 1H), 5.58 (dd, J = |
| | 11.0, 3.9 Hz, 1H), 5.51 (d, J = 15.8 Hz, 1H), 4.44 (d, J = 15.8 Hz, 1H), 4.39- |
| | 4.26 (m, 1H), 4.06 (t, J = 11.3 Hz, 1H), 3.98 (s, 3H), 3.75-3.64 (m, 2H), 3.63- |
| | 3.54 (m, 1H), 3.21 (hept, J = 6.8 Hz, 1H), 2.10 (s, 6H), 2.07-1.98 (m, 2H), |
| | 1.92-1.76 (m, 3H), 1.47-1.42 (m, 1H), 1.41 (d, J = 6.8 Hz, 3H), 1.39 (d, J = |
| | 6.8 Hz, 3H). |
| I-24 | ¹H NMR (400 MHz, Chloroform-d) δ 9.78 (s, 1H), 8.64 (s, 1H), 8.36 (s, 1H), |
| | 8.17 (d, J = 7.9 Hz, 1H), 7.82 (d, J = 7.6 Hz, 1H), 7.68 (t, J = 7.8 Hz, 1H), |
| | 7.19 (t, J = 7.7 Hz, 1H), 7.01 (d, J = 7.6 Hz, 2H), 6.61 (s, 1H), 6.17 (s, 1H), |
| | 5.39-5.29 (m, 2H), 4.32-4.18 (m, 2H), 4.09-3.96 (m, 1H), 2.13 (t, J = 6.9 |
| | Hz, 1H), 1.97 (s, 6H), 1.75-1.56 (m, 3H), 1.22-1.11 (m, 5H), 0.82 (s, 9H). |
| I-25 | ¹H NMR (400 MHz, Chloroform-d) δ 8.74 (s, 1H), 8.51 (s, 1H), 8.34 (s, 1H), |
| | 8.07 (d, J = 7.9 Hz, 1H), 7.86 (d, J = 7.6 Hz, 1H), 7.63 (t, J = 7.3 Hz, 1H), |
| | 7.24-7.18 (m, 1H), 7.06 (d, J = 7.5 Hz, 2H), 6.62 (s, 1H), 6.25 (s, 1H), 5.39 |
| | (d, J = 11.3 Hz, 1H), 5.27 (d, J = 14.8 Hz, 1H), 4.45-4.35 (m, 1H), 4.26 (t, |
| | J = 11.7 Hz, 1H), 4.15 (d, J = 14.9 Hz, 1H), 2.18-2.10 (m, 1H), 2.01 (s, 6H), |
| | 1.81 (d, J = 15.2 Hz, 1H), 1.53-1.40 (m, 2H), 1.19-1.10 (m, 4H), 0.48 (s, |
| | 3H), 0.34 (s, 1H), 0.24 (s, 1H), 0.13 (s, 1H). |
| I-27 | ¹H NMR (400 MHz, CDCl₃) δ 8.66 (s, 1H), 8.36-8.31 (m, 1H), 7.99 (d, J = |
| | 7.8 Hz, 1H), 7.82 (d, J = 7.5 Hz, 1H), 7.63 (t, J = 7.9 Hz, 1H), 7.24-7.17 (m, |
| | 1H), 7.03 (d, J = 7.6 Hz, 2H), 6.23-6.19 (m, 1H), 5.42-5.34 (m, 1H), 5.28 |
| | (d, J = 14.9 Hz, 1H), 4.28-4.18 (m, 2H), 4.09-4.01 (m, 1H), 2.56 (s, 3H), |
| | 2.01 (s, 6H), 1.87-1.78 (m, 1H), 1.76-1.64 (m, 2H), 1.28-1.11 (m, 3H), |
| | 1.03 (d, J = 8.2 Hz, 2H), 0.86-0.76 (m, 10H). |
| I-28 | ¹H NMR (400 MHz, CDCl₃) δ 8.79 (s, 1H), 8.32 (s, 1H), 7.95 (d, J = 7.9 Hz, |
| | 1H), 7.85 (d, J = 7.6 Hz, 1H), 7.60 (t, J = 7.8 Hz, 1H), 7.22 (t, J = 7.6 Hz, |
| | 1H), 7.07 (d, J = 7.6 Hz, 2H), 6.29 (s, 1H), 5.53-5.44 (m, 1H), 5.17 (d, J = |
| | 14.8 Hz, 1H), 4.35-4.20 (m, 3H), 2.56 (s, 3H), 2.03 (s, 6H), 1.88-1.72 (m, |
| | 3H), 1.29-1.19 (m, 2H), 1.10-1.00 (m, 2H), 0.61 (s, 9H). |
| I-29 | ¹H NMR (400 MHz, CDCl₃) δ 8.90 (t, J = 1.8 Hz, 1H), 8.66 (s, 1H), 8.05 (d, J = |
| | 7.9 Hz, 1H), 7.79 (dt, J = 7.7, 1.4 Hz, 1H), 7.64 (t, J = 7.8 Hz, 1H), 7.23 (d, |
| | J = 7.6 Hz, 1H), 7.07 (d, J = 7.7 Hz, 2H), 6.94 (s, 1H), 6.22 (s, 1H), 5.59 (dd, |
| | J = 10.5, 3.5 Hz, 1H), 5.50 (d, J = 16.0 Hz, 1H), 4.65 (d, J = 16.0 Hz, 1H), |
| | 4.34-4.21 (m, 1H), 4.18 (t, J = 11.0 Hz, 1H), 4.04 (s, 3H), 3.92 (dd, J = 11.9, |
| | 4.1 Hz, 1H), 3.82-3.72 (m, 1H), 3.41-3.31 (m, 1H), 3.30-3.26 (m, 1H), |
| | 3.25-3.19 (m, 1H), 2.05 (s, 6H), 1.87 (t, J = 12.2 Hz, 1H), 1.62-1.44 (m, |
| | 3H), 1.43 (d, J = 6.8 Hz, 3H), 1.41 (d, J = 6.9 Hz, 3H), 1.32 (qd, J = 11.9, 4.2 |
| | Hz, 1H), 0.95-0.77 (m, 2H). |
| I-31 | ¹H NMR (400 MHz, CDCl₃) δ 9.67 (s, 1H), 8.77 (s, 1H), 8.35 (s, 1H), 8.04 (d, |
| | J = 7.8 Hz, 1H), 7.78 (d, J = 7.5 Hz, 1H), 7.62 (t, J = 7.8 Hz, 1H), 7.21 (t, J = |
| | 7.7 Hz, 1H), 7.03 (d, J = 7.6 Hz, 2H), 6.20 (s, 1H), 5.52-5.42 (m, 1H), 5.38 |
| | (d, J = 15.2 Hz, 1H), 4.34 (d, J = 15.2 Hz, 1H), 4.28-4.12 (m, 2H), 3.94- |
| | 3.84 (m, 1H), 3.76 (d, J = 11.5 Hz, 1H), 3.34 (d, J = 11.8 Hz, 1H), 3.31-3.25 |
| | (m, 1H), 3.21 (t, J = 11.9 Hz, 1H), 2.31 (s, 3H), 1.99 (s, 6H), 1.89-1.76 (m, |
| | 1H), 1.60-1.41 (m, 3H), 1.41-1.35 (m, 6H), 1.35-1.24 (m, 1H), 0.96-0.73 |
| | (m, 2H). |
| I-32 | ¹H NMR (400 MHz, CDCl₃) δ 9.64 (s, 1H), 8.72 (s, 1H), 8.36 (s, 1H), 8.04 (d, |
| | J = 8.0 Hz, 1H), 7.81 (d, J = 7.5 Hz, 1H), 7.63 (t, J = 7.8 Hz, 1H), 7.20 (t, J = |
| | 7.7 Hz, 1H), 7.03 (d, J = 7.6 Hz, 2H), 6.18 (d, J = 2.3 Hz, 1H), 5.50-5.30 (m, |
| | 2H), 4.37-4.18 (m, 3H), 3.89 (d, J = 11.4 Hz, 1H), 3.34-3.13 (m, 2H), 3.10- |
| | 2.92 (m, 1H), 2.31 (d, J = 2.3 Hz, 3H), 2.00 (s, 6H), 1.97-1.86 (m, 1H), 1.73 |
| | (d, J = 13.5 Hz, 2H), 1.54-1.43 (m, 2H), 1.41-1.35 (m, 6H), 1.35-1.17 (m, |
| | 2H), 1.16-0.99 (m, 1H). |
| I-33 | ¹H NMR (400 MHz, CDCl₃) δ 9.51 (s, 1H), 8.72 (s, 1H), 8.33 (s, 1H), 7.77 (d, |
| | J = 6.7 Hz, 1H), 7.54-7.41 (m, 2H), 7.26-7.22 (m, 1H), 7.10 (d, J = 7.6 Hz, |
| | 2H), 6.22 (s, 1H), 5.57-5.39 (m, 2H), 4.45-4.32 (m, 1H), 4.29-4.15 (m, |
| | 2H), 3.81 (d, J = 11.3 Hz, 1H), 3.41 (t, J = 11.4 Hz, 1H), 3.35-3.16 (m, 2H), |
| | 2.31 (d, J = 2.1 Hz, 3H), 2.13 (s, 6H), 1.91-1.75 (m, 3H), 1.62-1.54 (m, |
| | 3H), 1.49-1.43 (m, 1H), 1.39-1.34 (m, 6H), 1.26-1.19 (m, 1H). |
| I-34 | ¹H NMR (400 MHz, CDCl₃) δ 8.72-8.69 (m, 1H), 8.38 (s, 1H), 8.03 (d, J = |
| | 7.9 Hz, 1H), 7.84 (dt, J = 7.7, 1.3 Hz, 1H), 7.65 (t, J = 7.8 Hz, 1H), 7.20 (t, J = |
| | 7.6 Hz, 1H), 7.02 (d, J = 7.6 Hz, 2H), 6.19 (s, 1H), 5.43-5.35 (m, 2H), 4.32- |
| | 4.21 (m, 2H), 4.09-3.99 (m, 1H), 3.33-3.21 (m, 1H), 2.30 (s, 3H), 2.00 (s, |
| | 6H), 1.92-1.80 (m, 1H), 1.78-1.65 (m, 4H), 1.38 (dd, J = 6.9, 4.3 Hz, 7H), |
| | 1.01 (s, 4H), 0.93-0.79 (m, 3H). |
| I-35 | ¹H NMR (400 MHz, CDCl₃) δ 8.71-8.67 (m, 1H), 8.42 (s, 1H), 7.98-7.92 |
| | (m, 1H), 7.81 (d, J = 7.6 Hz, 1H), 7.60 (t, J = 7.8 Hz, 1H), 7.21 (t, J = 7.6 Hz, |
| | 1H), 7.05 (d, J = 7.6 Hz, 2H), 6.64-6.61 (m, 1H), 6.22 (s, 1H), 5.43 (dd, J = |
| | 11.1, 4.2 Hz, 1H), 5.29 (d, J = 14.9 Hz, 1H), 4.45-4.36 (m, 1H), 4.35-4.24 |
| | (m, 2H), 3.22-3.11 (m, 1H), 2.27-2.12 (m, 1H), 2.00 (s, 6H), 1.97-1.83 (m, |
| | 1H), 1.40 (d, J = 6.9 Hz, 6H), 1.23 (d, 3H), 0.96 (d, 3H). |
| I-38 | ¹H NMR (400 MHz, Chloroform-d) δ 8.77 (t, J = 1.7 Hz, 1H), 8.56 (s, 1H), |
| | 8.40 (s, 1H), 8.08-8.00 (m, 1H), 7.84 (dt, J = 7.7, 1.3 Hz, 1H), 7.63 (t, J = |
| | 7.8 Hz, 1H), 7.21 (t, J = 7.6 Hz, 1H), 7.06 (d, J = 7.6 Hz, 2H), 6.63 (d, J = 1.1 |
| | Hz, 1H), 6.26 (s, 1H), 5.44 (dd, J = 11.0, 3.9 Hz, 1H), 5.26 (d, J = 14.9 Hz, |
| | 1H), 4.38-4.24 (m, 2H), 4.22-4.15 (m, 1H), 3.23-3.08 (m, 1H), 2.00 (s, |
| | 6H), 1.76 (dd, J = 15.3, 8.7 Hz, 1H), 1.63-1.59 (m, 1H), 1.40 (d, J = 6.9 Hz, |
| | 6H), 1.01-0.84 (m, 2H), 0.62 (s, 3H), 0.60-0.52 (m, 6H). |
| I-39 | ¹H NMR (400 MHz, Chloroform-d) δ 8.77 (d, J = 1.8 Hz, 1H), 8.46 (s, 1H), |
| | 8.40 (d, J = 1.3 Hz, 1H), 8.08-8.01 (m, 1H), 7.87-7.81 (m, 1H), 7.63 (t, J = |
| | 7.9 Hz, 1H), 7.21 (t, J = 7.6 Hz, 1H), 7.06 (d, J = 7.6 Hz, 2H), 6.63 (d, J = 1.1 |
| | Hz, 1H), 6.26 (d, J = 1.3 Hz, 1H), 5.44 (dd, J = 11.0, 3.9 Hz, 1H), 5.26 (d, J = |
| | 14.9 Hz, 1H), 4.38-4.24 (m, 2H), 4.24-4.14 (m, 1H), 3.22-3.10 (m, 1H), |
| | 2.00 (s, 6H), 1.76 (dd, J = 15.3, 8.7 Hz, 1H), 1.63-1.59 (m, 1H), 1.40 (d, J = |
| | 6.9 Hz, 6H), 1.01-0.83 (m, 2H), 0.62 (s, 3H), 0.60-0.52 (m, 6H). |
| I-40 | ¹H NMR (400 MHz, Chloroform-d) δ 9.00 (t, J = 1.7 Hz, 1H), 8.79 (s, 1H), |
| | 8.48 (s, 1H), 8.07 (d, J = 7.8 Hz, 1H), 7.92 (dt, J = 7.8, 1.3 Hz, 1H), 7.67 (t, J = |
| | 7.8 Hz, 1H), 7.24-7.18 (m, 1H), 7.06 (d, J = 7.6 Hz, 2H), 6.46 (s, 1H), 6.21 |
| | (s, 1H), 5.56 (dd, J = 11.0, 3.6 Hz, 1H), 5.44 (d, J = 15.8 Hz, 1H), 4.29 (d, J = |
| | 15.8 Hz, 1H), 4.12-4.02 (m, 1H), 3.97 (t, J = 11.3 Hz, 1H), 3.90 (s, 3H), 3.15 |
| | (hept, J = 6.8 Hz, 1H), 2.46 (s, 1H), 2.04 (s, 6H), 1.98 (dd, J = 15.4, 10.5 Hz, |
| | 1H), 1.82 (dd, J = 15.5, 3.2 Hz, 1H), 1.64-1.56 (overlapped with water m, |
| | 6H), 1.41-1.34 (m, 6H) |
| I-41 | ¹H NMR (400 MHz, CDCl₃) δ 8.88 (t, J = 1.8 Hz, 1H), 8.49 (s, 1H), 7.97 |
| | (d, J = 7.8 Hz, 1H), 7.88-7.83 (m, 1H), 7.64 (t, J = 7.8 Hz, 1H), 7.20 (t, J = 7.6 |
| | Hz, 1H), 7.02 (d, J = 7.6 Hz, 2H), 6.48 (s, 1H), 6.16 (s, 1H), 5.54-5.45 (m, |
| | 2H), 4.29 (d, J = 15.6 Hz, 1H), 4.09-3.99 (m, 2H), 3.90 (s, 3H), 3.16 (p, J = |
| | 6.8 Hz, 1H), 2.00 (s, 6H), 1.92-1.81 (m, 2H), 1.79-1.65 (m, 4H), 1.44-1.34 |
| | (m, 8H), 1.10-0.99 (m, 5H). |
| I-42 | ¹H NMR (400 MHz, Chloroform-d) δ 8.68 (t, J = 1.8 Hz, 1H), 8.35 (s, 1H), |
| | 8.11 (d, J = 7.9 Hz, 1H), 7.80 (dt, J = 7.7, 1.4 Hz, 1H), 7.63 (t, J = 7.8 Hz, |
| | 1H), 7.20 (t, J = 7.6 Hz, 1H), 7.03 (d, J = 7.6 Hz, 2H), 6.62 (s, 1H), 6.19 (s, |
| | 1H), 5.37 (dd, J = 10.1, 3.0 Hz, 1H), 5.29 (d, J = 15.0 Hz, 1H), 4.31 (d, J = |
| | 14.9 Hz, 1H), 4.26-4.09 (m, 2H), 2.19-2.07 (m, 1H), 1.99 (s, 6H), 1.75- |
| | 1.57 (m, 4H), 1.51-1.39 (m, 3H), 1.19-1.11 (m, 5H), 1.07-0.98 (m, 2H), |
| | 0.98-0.83 (m, 2H), 0.44-0.32 (m, 1H). |
| I-43 | ¹H NMR (400 MHz, Chloroform-d) δ 9.38 (s, 1H), 8.72 (t, J = 1.8 Hz, 1H), |
| | 8.40 (s, 1H), 8.14 (d, J = 8.1 Hz, 1H), 7.88 (dt, J = 7.7, 1.4 Hz, 1H), 7.67 (t, J = |
| | 7.8 Hz, 1H), 7.19 (t, J = 7.6 Hz, 1H), 7.03 (d, J = 7.6 Hz, 2H), 6.62 (d, J = |
| | 1.0 Hz, 1H), 6.18 (s, 1H), 5.41 (dd, J = 11.4, 4.0 Hz, 1H), 5.37-5.28 (m, 1H), |
| | 4.27-4.16 (m, 2H), 4.09-3.97 (m, 1H), 3.23-3.09 (m, 1H), 2.45 (s, 1H), |
| | 2.00 (s, 6H), 1.81 (dd, J = 15.6, 3.3 Hz, 1H), 1.61-1.55 (m, 7H), 1.40 (d, J = |
| | 6.9 Hz, 6H). |
| I-44 | ¹H NMR (400 MHz, CDCl₃) δ 8.63 (t, J = 1.8 Hz, 1H), 8.36 (s, 1H), 8.18 |
| | (d, J = 7.9 Hz, 1H), 7.80 (dt, J = 7.7, 1.3 Hz, 1H), 7.66 (t, J = 7.8 Hz, 1H), 7.19 (t, |
| | J = 7.6 Hz, 1H), 7.00 (d, J = 7.6 Hz, 2H), 6.60 (s, 1H), 6.14 (s, 1H), 5.38- |
| | 5.33 (m, 1H), 5.31 (d, J = 15.1 Hz, 1H), 4.34-4.20 (m, 3H), 3.94-3.86 (m, |
| | 1H), 3.22 (td, J = 11.2, 3.6 Hz, 1H), 3.06-2.97 (m, 1H), 2.17-2.09 (m, 1H), |
| | 2.04-1.97 (m, 1H), 1.95 (s, 6H), 1.76-1.65 (m, 2H), 1.51-1.42 (m, 2H), |
| | 1.36 (tt, J = 12.4, 4.1 Hz, 1H), 1.29-1.22 (m, 1H), 1.18-1.04 (m, 5H). |
| I-45 | ¹H NMR (400 MHz, CDCl₃) δ 8.64 (t, J = 1.8 Hz, 1H), 8.36 (s, 1H), 8.14 |
| | (d, J = 7.9 Hz, 1H), 7.81 (dt, J = 7.7, 1.4 Hz, 1H), 7.64 (t, J = 7.8 Hz, 1H), 7.19 (t, |
| | J = 7.6 Hz, 1H), 7.01 (d, J = 7.6 Hz, 2H), 6.61 (s, 1H), 6.15 (s, 1H), 5.42- |
| | 5.30 (m, 2H), 4.35-4.24 (m, 2H), 4.24-4.13 (m, 1H), 3.78-3.69 (m, 1H), |
| | 3.67-3.54 (m, 2H), 2.17-2.10 (m, 1H), 2.10-2.01 (m, 1H), 1.96 (s, 6H), |
| | 1.83-1.78 (m, 1H), 1.78-1.69 (m, 3H), 1.20-1.09 (m, 5H). |
| I-46 | ¹H NMR (400 MHz, CDCl₃) δ 8.62 (t, J = 1.8 Hz, 1H), 8.34 (s, 1H), 7.78 (dt, |
| | J = 7.6, 1.4 Hz, 1H), 7.73 (d, J = 7.9 Hz, 1H), 7.55 (t, J = 7.8 Hz, 1H), 7.22 (t, |
| | J = 7.6 Hz, 1H), 7.06 (d, J = 7.6 Hz, 2H), 6.63 (s, 1H), 6.18 (s, 1H), 5.45- |
| | 5.33 (m, 2H), 4.33-4.16 (m, 3H), 3.71-3.59 (m, 2H), 3.59-3.49 (m, 1H), |
| | 2.17-2.09 (m, 1H), 2.04 (s, 6H), 2.03-1.98 (m, 1H), 1.98-1.91 (m, 1H), |
| | 1.89-1.73 (m, 3H), 1.42-1.31 (m, 1H), 1.20-1.09 (m, 4H). |
| I-47 | ¹H NMR (400 MHz, CDCl₃) δ 8.63 (t, J = 1.8 Hz, 1H), 8.37 (s, 1H), 8.17 |
| | (d, J = 7.9 Hz, 1H), 7.83 (dt, J = 7.7, 1.3 Hz, 1H), 7.68 (t, J = 7.8 Hz, 1H), 7.18 (t, |
| | J = 7.6 Hz, 1H), 7.00 (d, J = 7.6 Hz, 2H), 6.61 (s, 1H), 6.15 (s, 1H), 5.38- |
| | 5.32 (m, 2H), 4.31-4.20 (m, 2H), 4.09-3.99 (m, 1H), 2.18-2.09 (m, 1H), |
| | 1.96 (s, 6H), 1.90-1.80 (m, 1H), 1.68-1.50 (m, 7H), 1.34 (td, J = 12.7, 4.6 |
| | Hz, 1H), 1.21-1.11 (m, 4H), 1.07-0.96 (m, 4H). |
| I-48 | ¹H NMR (400 MHz, CDCl₃) δ 8.77 (t, J = 1.8 Hz, 1H), 8.35 (s, 1H), 7.87- |
| | 7.79 (m, 2H), 7.58 (t, J = 7.8 Hz, 1H), 7.22 (t, J = 7.6 Hz, 1H), 7.07 (d, J = |
| | 7.6 Hz, 2H), 6.24 (s, 1H), 5.48 (dd, J = 11.3, 4.4 Hz, 1H), 5.32 (d, J = 15.2 |
| | Hz, 1H), 4.46-4.37 (m, 1H), 4.32 (d, J = 15.2 Hz, 1H), 4.24 (t, J = 11.5 Hz, |
| | 1H), 3.28 (hept, J = 6.9 Hz, 1H), 2.31 (s, 3H), 2.25-2.12 (m, 1H), 2.03 (s, |
| | 6H), 1.99-1.86 (m, 1H), 1.37 (dd, J = 6.9, 4.8 Hz, 6H), 1.24 (d, 3H), 0.97 (d, |
| | 3H). |
| I-51 | ¹H NMR (400 MHz, CDCl₃) δ 8.70 (t, J = 1.8 Hz, 1H), 8.36 (s, 1H), 8.00- |
| | 7.95 (m, 1H), 7.84-7.80 (m, 1H), 7.61 (t, J = 7.8 Hz, 1H), 7.21 (t, J = 7.6 Hz, |
| | 1H), 7.06 (d, J = 7.6 Hz, 2H), 6.62 (s, 1H), 6.23 (s, 1H), 5.41 (dd, J = 11.3, |
| | 4.3 Hz, 1H), 5.27 (d, J = 14.9 Hz, 1H), 4.45-4.36 (m, 1H), 4.32-4.23 (m, |
| | 2H), 2.25-2.09 (m, 2H), 2.01 (s, 6H), 1.96-1.83 (m, 1H), 1.27-1.19 (m, |
| | 3H), 1.19-1.10 (m, 4H), 0.95 (m, 3H). |
| I-54 | ¹H NMR (400 MHz, CDCl₃) δ 8.69 (t, J = 1.8 Hz, 1H), 8.63 (s, 1H), 8.40 (s, |
| | 1H), 7.88 (dt, J = 8.1, 1.3 Hz, 1H), 7.80 (dt, J = 7.8, 1.4 Hz, 1H), 7.57 (t, J = |
| | 7.8 Hz, 1H), 7.22 (t, J = 7.6 Hz, 1H), 7.07 (d, J = 7.6 Hz, 2H), 6.62 (d, J = 1.0 |
| | Hz, 1H), 6.28 (s, 1H), 5.44 (dd, J = 11.5, 4.6 Hz, 1H), 5.18 (d, J = 14.7 Hz, |
| | 1H), 4.69-4.55 (m, 1H), 4.25 (t, J = 11.7 Hz, 1H), 4.18 (d, J = 14.7 Hz, 1H), |
| | 3.16 (pd, J = 6.9, 1.1 Hz, 1H), 2.47 (d, J = 16.1 Hz, 1H), 2.02 (s, 6H), 1.61 |
| | (dd, J = 16.3, 9.8 Hz, 1H), 1.40 (d, J = 7.0 Hz, 6H), 0.96-0.82 (m, 2H), 0.78- |
| | 0.64 (m, 1H), 0.51-0.37 (m, 1H). |
| I-55 | ¹H NMR (400 MHz, CDCl₃) δ 9.03 (s, 1H), 8.72 (t, J = 1.8 Hz, 1H), 8.41 (s, |
| | 1H), 8.02 (dt, J = 8.2, 1.3 Hz, 1H), 7.80 (dt, J = 7.7, 1.3 Hz, 1H), 7.60 (t, J = |
| | 7.8 Hz, 1H), 7.21 (t, J = 7.6 Hz, 1H), 7.05 (d, J = 7.6 Hz, 2H), 6.63 (d, J = 1.0 |
| | Hz, 1H), 6.24 (s, 1H), 5.41 (dd, J = 11.1, 4.2 Hz, 1H), 5.30 (d, J = 14.9 Hz, |
| | 1H), 4.34 (d, J = 14.9 Hz, 1H), 4.29 (t, J = 11.5 Hz, 1H), 4.23-4.11 (m, 1H), |
| | 3.16 (pd, J = 6.9, 1.0 Hz, 1H), 1.99 (s, 6H), 1.97-1.91 (m, 1H), 1.90-1.74 |
| | (m, 2H), 1.73-1.62 (m, 2H), 1.58-1.50 (m, 1H), 1.47-1.41 (m, 2H), 1.40 (d, |
| | J = 7.0 Hz, 6H), 0.68 (s, 3H). |
| I-56 | ¹H NMR (400 MHz, CDCl₃) δ 8.64 (t, J = 1.8 Hz, 1H), 8.40 (s, 1H), 8.09- |
| | 8.04 (m, 1H), 7.82 (dt, J = 7.7, 1.3 Hz, 1H), 7.62 (t, J = 7.8 Hz, 1H), 7.20 (t, J = |
| | 7.6 Hz, 1H), 7.03 (d, J = 7.6 Hz, 2H), 6.62 (d, J = 1.0 Hz, 1H), 6.20 (s, 1H), |
| | 5.39-5.30 (m, 2H), 4.31-4.20 (m, 2H), 4.07-3.97 (m, 1H), 3.22-3.10 (m, |
| | 1H), 2.15 (p, J = 7.8 Hz, 1H), 1.99 (s, 6H), 1.95-1.85 (m, 1H), 1.85-1.75 |
| | (m, 1H), 1.72-1.64 (m, 3H), 1.62-1.54 (m, 2H), 1.40 (d, J = 6.9 Hz, 6H), |
| | 1.18-1.05 (m, 1H). |
| I-57 | ¹H NMR (400 MHz, CDCl₃) δ 8.64 (t, J = 1.8 Hz, 1H), 8.42 (s, 1H), 8.09 |
| | (d, J = 7.9 Hz, 1H), 7.81 (dt, J = 7.8, 1.3 Hz, 1H), 7.63 (t, J = 7.8 Hz, 1H), 7.19 (t, |
| | J = 7.6 Hz, 1H), 7.01 (d, J = 7.6 Hz, 2H), 6.62 (d, J = 1.0 Hz, 1H), 6.13 (s, |
| | 1H), 5.45-5.33 (m, 2H), 4.33-4.07 (m, 3H), 3.21-3.12 (m, 1H), 1.97 (s, |
| | 6H), 1.40 (d, J = 6.9 Hz, 6H), 1.37-1.25 (m, 2H), 0.55-0.47 (m, 2H), 0.34- |
| | 0.25 (m, 1H), 0.15-0.07 (m, 1H), −0.29-−0.37 (m, 1H). |
| I-58 | ¹H NMR (400 MHz, Chloroform-d) δ 10.13 (s, 1H), 8.64 (s, 1H), 8.32 (s, 1H), |
| | 7.82 (d, J = 7.5, 1.5 Hz, 1H), 7.63 (d, J = 8.0 Hz, 1H), 7.55 (t, J = 7.7 Hz, |
| | 1H), 7.25 (t, 1H), 7.07 (d, J = 7.6 Hz, 2H), 6.62 (d, J = 1.2 Hz, 1H), 6.20 (d, J = |
| | 1.3 Hz, 1H), 5.41-5.28 (m, 2H), 4.56 (d, J = 15.2 Hz, 1H), 4.43 (t, J = 11.5 |
| | Hz, 1H), 4.33 (d, J = 11.8, 7.6, 3.7 Hz, 1H), 3.77 (p, J = 7.2 Hz, 1H), 3.57 (t, J = |
| | 10.7, 7.7 Hz, 1H), 3.50-3.47 (m, 1H), 2.17-2.06 (m, 3H), 2.04 (s, 6H), |
| | 1.86-1.75 (m, 2H), 1.68-1.63 (m, 1H), 1.50-1.41 (m, 1H), 1.19-1.09 (m, |
| | 4H). |
| I-59 | ¹H NMR (400 MHz, Chloroform-d) δ 8.63 (s, 1H), 8.31 (s, 1H), 7.81 (d, J = |
| | 7.4, 1.5 Hz, 1H), 7.62-7.49 (m, 2H), 7.24 (t, 1H), 7.06 (d, J = 7.6 Hz, 2H), |
| | 6.62 (s, 1H), 6.19 (s, 1H), 5.40-5.29 (m, 2H), 4.56 (d, J = 15.2 Hz, 1H), 4.43 |
| | (t, J = 11.5 Hz, 1H), 4.36-4.27 (m, 1H), 3.77 (p, J = 7.2 Hz, 1H), 3.56 (t, J = |
| | 10.7, 7.8 Hz, 1H), 3.50-3.43 (m, 1H), 2.18-2.06 (m, 3H), 2.04 (s, 6H), 1.85- |
| | 1.76 (m, 2H), 1.69-1.62 (m, 1H), 1.50-1.40 (m, 1H), 1.18-1.11 (m, 4H). |
| I-60 | ¹H NMR (400 MHz, CDCl₃) δ 8.78 (s, 1H), 8.41 (s, 1H), 8.06 (d, J = 1.4 Hz, |
| | 1H), 7.83 (dt, J = 7.6, 1.4 Hz, 1H), 7.64 (t, J = 7.8 Hz, 1H), 7.22 (t, J = 7.6 |
| | Hz, 1H), 7.07 (d, J = 7.6 Hz, 2H), 6.63 (s, 1H), 6.29 (s, 1H), 5.50 (dd, J = |
| | 11.1, 3.9 Hz, 1H), 5.23 (d, J = 14.8 Hz, 1H), 4.42-4.33 (m, 2H), 4.28 (t, J = |
| | 11.4 Hz, 1H), 3.22-3.11 (m, 1H), 2.11-1.93 (m, 8H), 1.40 (d, J = 6.9 Hz, |
| | 6H), 0.98 (s, 3H), 0.76 (s, 3H). |
| I-61 | ¹H NMR (400 MHz, Chloroform-d) δ 9.27 (s, 1H), 8.69 (t, J = 1.8 Hz, 1H), |
| | 8.35 (s, 1H), 8.10 (dd, J = 7.8, 1.8 Hz, 1H), 7.83 (dt, J = 7.7, 1.4 Hz, 1H), |
| | 7.64 (t, J = 7.8 Hz, 1H), 7.19 (t, J = 7.6 Hz, 1H), 7.03 (d, J = 7.6 Hz, 2H), |
| | 6.62 (s, 1H), 6.20 (s, 1H), 5.38 (dd, J = 11.2, 4.1 Hz, 1H), 5.32 (d, J = 15.0 |
| | Hz, 1H), 4.35-4.19 (m, 2H), 4.15-4.05 (m, 1H), 2.19-2.09 (m, 1H), 1.99 (s, |
| | 6H), 1.92 (ddd, J = 14.1, 10.5, 3.5 Hz, 1H), 1.73-1.63 (m, 2H), 1.53-1.50 |
| | (m, 1H), 1.43-1.36 (m, 2H), 1.31-1.18 (m, 2H), 1.18-1.10 (m, 4H), 1.10- |
| | 1.01 (m, 1H), 0.89-0.76 (m, 1H), 0.52-0.41 (m, 1H). |
| I-63 | ¹H NMR (400 MHz, CDCl₃) δ 9.17 (t, J = 1.6 Hz, 1H), 8.10 (d, J = 7.8 Hz, |
| | 1H), 7.86 (dt, J = 7.7, 1.3 Hz, 1H), 7.65 (t, J = 7.8 Hz, 1H), 7.43 (dd, J = 9.7, |
| | 0.9 Hz, 1H), 7.23-7.17 (m, 1H), 7.07 (d, J = 7.6 Hz, 2H), 6.65 (t, J = 0.9 Hz, |
| | 1H), 6.24 (s, 1H), 5.67 (dd, J = 11.4, 4.3 Hz, 1H), 5.47 (dd, J = 16.3, 1.6 Hz, |
| | 1H), 4.32 (d, J = 14.9 Hz, 1H), 4.22-4.12 (m, 1H), 3.87 (t, J = 11.5 Hz, 1H), |
| | 3.15-3.01 (m, 1H), 2.01 (s, 6H), 1.78 (dd, J = 15.2, 8.8 Hz, 1H), 1.35 (dd, J = |
| | 7.0, 2.1 Hz, 6H), 0.68 (s, 9H), (1H labile missing, 1H overlap H2O). |
| I-64 | ¹H NMR (400 MHz, Chloroform-d) δ 8.78 (t, J = 1.8 Hz, 1H), 8.57 (s, 1H), |
| | 8.34 (s, 1H), 8.05 (dt, J = 8.2, 1.5 Hz, 1H), 7.85 (dt, J = 7.8, 1.3 Hz, 1H), 7.64 |
| | (t, J = 7.8 Hz, 1H), 7.24-7.16 (m, 1H), 7.06 (d, J = 7.6 Hz, 2H), 6.24 (s, 1H), |
| | 5.46 (dd, J = 11.0, 3.9 Hz, 1H), 5.36 (d, J = 15.2 Hz, 1H), 4.34 (d, J = 15.2 |
| | Hz, 1H), 4.24 (t, J = 11.4 Hz, 1H), 4.18-4.08 (m, 1H), 3.33-3.22 (m, 1H), |
| | 2.31 (s, 3H), 2.03 (s, 6H), 1.98-1.88 (m, 1H), 1.74-1.66 (m, 2H), 1.48-1.40 |
| | (m, 2H), 1.37 (dd, J = 6.9, 4.4 Hz, 6H), 1.28-1.19 (m, 2H), 1.09-1.01 (m, |
| | 1H), 0.92-0.77 (m, 2H), 0.50-0.38 (m, 1H). |
| I-65 | ¹H NMR (400 MHz, Chloroform-d) δ 8.79 (t, J = 1.8 Hz, 1H), 8.41 (s, 1H), |
| | 8.09 (dt, J = 8.1, 1.3 Hz, 1H), 7.85 (dt, J = 7.8, 1.4 Hz, 1H), 7.63 (t, J = 7.8 |
| | Hz, 1H), 7.24 (d, J = 7.6 Hz, 1H), 7.12 (d, J = 7.7 Hz, 1H), 7.06 (d, J = 7.5 |
| | Hz, 1H), 6.63 (d, J = 1.0 Hz, 1H), 5.49 (dd, J = 11.2, 4.4 Hz, 1H), 5.27 (d, J = |
| | 14.8 Hz, 1H), 4.43-4.32 (m, 2H), 4.26-4.19 (m, 1H), 3.24-3.09 (m, 1H), |
| | 2.26-2.14 (m, 2H), 1.87-1.81 (m, 1H), 1.80 (s, 3H), 1.80-1.75 (m, 1H), |
| | 1.74 (s, 3H), 1.59 (dd, J = 15.1, 1.5 Hz, 1H), 1.40 (d, J = 6.9 Hz, 6H), 0.81 |
| | (dd, J = 6.6, 3.2 Hz, 6H), 0.61 (s, 9H). |
| I-68 | ¹H NMR (400 MHz, CDCl₃) δ 9.03 (t, J = 1.9 Hz, 1H), 8.24 (d, J = 8.4 Hz, |
| | 1H), 7.83-7.78 (m, 1H), 7.57 (t, J = 7.7 Hz, 1H), 7.54-7.48 (m, 1H), 7.31- |
| | 7.26 (m, 1H), 7.09 (d, J = 7.6 Hz, 2H), 6.20 (s, 1H), 5.64-5.55 (m, 1H), 5.50 |
| | (dd, J = 10.9, 4.2 Hz, 1H), 4.91-4.83 (m, 1H), 4.39-4.30 (m, 1H), 4.14 (t, J = |
| | 11.3 Hz, 1H), 4.09 (s, 3H), 3.67-3.43 (m, 4H), 2.07 (s, 6H), 1.66 (d, J = |
| | 7.0 Hz, 3H), 1.65-1.62 (m, 3H), 1.17 (d, J = 4.8 Hz, 3H), 1.16 (d, J = 4.8 Hz, |
| | 3H). |
| I-69 | ¹H NMR (400 MHz, CDCl₃) δ 9.19 (t, J = 1.8 Hz, 1H), 8.02-7.93 (m, 2H), |
| | 7.86 (dt, J = 7.7, 1.4 Hz, 1H), 7.65 (t, J = 7.8 Hz, 1H), 7.22 (t, J = 7.6 Hz, |
| | 1H), 7.06 (d, J = 7.6 Hz, 2H), 6.20 (s, 1H), 5.62 (dd, J = 11.1, 4.2 Hz, 1H), |
| | 5.57-5.50 (m, 1H), 4.46-4.37 (m, 1H), 4.27-4.18 (m, 1H), 3.99 (s, 3H), |
| | 3.75 (t, J = 11.3 Hz, 1H), 3.34 (p, J = 6.9 Hz, 1H), 2.00 (s, 6H), 1.73 (dd, J = |
| | 15.0, 8.8 Hz, 2H), 1.54 (d, J = 6.9 Hz, 3H), 1.51 (d, J = 6.9 Hz, 3H), 0.69 (s, |
| | 9H). |
| I-70 | ¹H NMR (400 MHz, CDCl₃) δ 8.62 (t, J = 1.8 Hz, 1H), 8.42 (s, 1H), 8.12 |
| | (d, J = 8.0 Hz, 1H), 7.79 (dt, J = 7.7, 1.4 Hz, 1H), 7.65 (t, J = 7.8 Hz, 1H), 7.19 (t, |
| | J = 7.6 Hz, 1H), 7.01 (d, J = 7.6 Hz, 2H), 6.62 (d, J = 1.0 Hz, 1H), 6.17 (s, |
| | 1H), 5.40-5.28 (m, 2H), 4.35-4.22 (m, 2H), 4.10-3.99 (m, 1H), 3.22-3.10 |
| | (m, 1H), 1.97 (s, 6H), 1.90-1.68 (m, 3H), 1.40 (d, J = 6.9 Hz, 6H), 1.24- |
| | 1.14 (m, 1H), 0.93 (s, 3H), 0.22-0.10 (m, 4H). |
| I-72 | ¹H NMR (400 MHz, CDCl₃) δ 8.88 (t, J = 1.8 Hz, 1H), 8.48 (s, 1H), 7.94 |
| | (d, J = 7.9 Hz, 1H), 7.82 (dt, J = 7.6, 1.4 Hz, 1H), 7.62 (t, J = 7.8 Hz, 1H), 7.21 (t, |
| | J = 7.6 Hz, 1H), 7.03 (d, J = 7.6 Hz, 2H), 6.46 (s, 1H), 6.18 (s, 1H), 5.53- |
| | 5.41 (m, 2H), 4.29 (d, J = 15.7 Hz, 1H), 4.10-4.00 (m, 2H), 3.89 (s, 3H), 3.20- |
| | 3.10 (m, 1H), 2.01 (s, 6H), 1.82-1.75 (m, 2H), 1.38 (dd, J = 10.2, 6.8 Hz, |
| | 6H), 1.28-1.20 (m, 1H), 0.97-0.87 (m, 4H), 0.24-0.11 (m, 4H). |
| I-73 | ¹H NMR (400 MHz, CDCl₃) δ 9.03 (t, J = 1.8 Hz, 1H), 8.61 (s, 1H), 8.00 (dt, |
| | J = 8.1, 1.4 Hz, 1H), 7.86 (dt, J = 7.7, 1.4 Hz, 1H), 7.64 (t, J = 7.8 Hz, 1H), |
| | 7.23 (t, J = 7.6 Hz, 1H), 7.07 (d, J = 7.7 Hz, 2H), 6.24 (s, 1H), 5.62 (dd, J = |
| | 11.1, 4.1 Hz, 1H), 5.44 (d, J = 16.0 Hz, 1H), 4.43 (d, J = 16.0 Hz, 1H), 4.24- |
| | 4.13 (m, 1H), 4.04-3.96 (m, 1H), 3.95 (s, 3H), 3.32 (hept, J = 6.9 Hz, 1H), |
| | 2.02 (s, 6H), 1.76 (dd, J = 15.1, 8.8 Hz, 1H), 1.60 (dd, J = 15.0, 1.7 Hz, 1H), |
| | 1.52 (d, J = 6.8 Hz, 3H), 1.49 (d, J = 6.8 Hz, 3H), 0.68 (s, 9H). |
| I-74 | ¹H NMR (400 MHz, CDCl₃) δ 9.29 (s, 1H), 8.71 (s, 1H), 8.59 (t, J = 1.7 Hz, |
| | 1H), 8.11 (dt, J = 8.1, 1.3 Hz, 1H), 7.81 (dt, J = 7.7, 1.3 Hz, 1H), 7.62 (t, J = |
| | 7.8 Hz, 1H), 7.21 (t, J = 7.6 Hz, 1H), 7.05 (d, J = 7.6 Hz, 2H), 6.24 (s, 1H), |
| | 5.26 (dd, J = 11.3, 4.4 Hz, 1H), 5.21 (d, J = 14.3 Hz, 1H), 4.50 (t, J = 11.5 |
| | Hz, 1H), 4.41 (d, J = 14.3 Hz, 1H), 4.27-4.16 (m, 1H), 3.52 (hept, J = 6.9 |
| | Hz, 1H), 1.99 (s, 6H), 1.86 (dd, J = 15.3, 8.8 Hz, 1H), 1.62 (dd, J = 15.3, 1.7 |
| | Hz, 1H), 1.54 (d, J = 6.8 Hz, 3H), 1.52 (d, J = 6.8 Hz, 3H), 0.63 (s, 9H). |
| I-75 | ¹H NMR (400 MHz, CDCl₃) δ 9.02 (t, J = 1.8 Hz, 1H), 8.58 (s, 1H), 7.99 (dd, |
| | J = 7.9, 1.8 Hz, 1H), 7.87 (dt, J = 7.7, 1.3 Hz, 1H), 7.63 (t, J = 7.8 Hz, 1H), |
| | 7.22 (t, J = 7.6 Hz, 1H), 7.06 (d, J = 7.6 Hz, 2H), 6.23 (s, 1H), 5.56 (dd, J = |
| | 11.2, 4.2 Hz, 1H), 5.45 (d, J = 15.9 Hz, 1H), 4.43-4.33 (m, 1H), 4.26 (d, J = |
| | 16.0 Hz, 1H), 4.04-3.96 (m, 1H), 3.94 (s, 3H), 3.31 (hept, J = 6.8 Hz, 1H), |
| | 2.02 (s, 6H), 1.84 (d, J = 14.9 Hz, 1H), 1.51 (d, J = 6.8 Hz, 3H), 1.48 (d, J = |
| | 6.8 Hz, 3H), 1.47-1.38 (m, 1H), 0.56 (s, 3H), 0.43-0.35 (m, 1H), 0.34-0.25 |
| | (m, 1H), 0.20-0.13 (m, 1H), 0.09-0.01 (m, 1H). |
| I-76 | ¹H NMR (400 MHz, Chloroform-d) δ 8.64 (t, J = 1.8 Hz, 1H), 8.47-8.10 (m, |
| | 2H), 7.99-7.91 (m, 1H), 7.81 (dt, J = 7.7, 1.3 Hz, 1H), 7.59 (t, J = 7.8 Hz, |
| | 1H), 7.21 (t, J = 7.6 Hz, 1H), 7.06 (d, J = 7.6 Hz, 2H), 6.64 (d, J = 1.0 Hz, |
| | 1H), 6.27 (s, 1H), 5.44-5.31 (m, 2H), 4.38-4.30 (m, 2H), 4.30-4.23 (m, |
| | 1H), 3.24-3.09 (m, 1H), 2.77 (s, 3H), 2.16-2.09 (m, 1H), 2.08-2.03 (m, |
| | 1H), 2.03-1.98 (m, 3H), 1.90 (d, J = 15.3 Hz, 1H), 1.83-1.68 (m, 2H), 1.56 |
| | (s, 4H), 1.40 (d, J = 6.9 Hz, 6H), 1.10-0.99 (m, 1H), 0.86-0.79 (m, 1H). |
| I-77 | ¹H NMR (400 MHz, Chloroform-d) δ 9.46 (s, 1H), 8.99 (s, 1H), 7.82 (d, J = |
| | 7.5 Hz, 1H), 7.74 (s, 1H), 7.57 (t, J = 7.7 Hz, 1H), 7.41 (d, J = 9.6, 1.0 Hz, |
| | 1H), 7.23 (t, J = 7.6 Hz, 1H), 7.06 (d, J = 7.6 Hz, 2H), 6.62 (s, 1H), 6.19 (s, |
| | 1H), 5.57 (dd, J = 16.2, 1.8 Hz, 1H), 5.51 (dd, J = 11.3, 4.3 Hz, 1H), 4.57 (d, |
| | J = 16.2, 1.7 Hz, 1H), 4.33-4.25 (m, 1H), 4.12 (t, J = 11.5 Hz, 1H), 3.70- |
| | 3.59 (m, 2H), 3.52 (hept, J = 6.0 Hz, 1H), 3.07 (hept, J = 6.9 Hz, 1H), 2.05 (s, |
| | 6H), 1.34 (dd, J = 6.9, 1.5 Hz, 6H), 1.14 (d, J = 6.0 Hz, 3H), 1.09 (d, J = 6.0 |
| | Hz, 3H). |
| I-78 | ¹H NMR (400 MHz, Chloroform-d) δ 9.14 (s, 1H), 8.48 (s, 1H), 8.07 (dt, J = |
| | 8.0, 2.0, 1.1 Hz, 1H), 7.86 (dt, J = 7.6, 1.3 Hz, 1H), 7.63 (t, J = 7.8 Hz, 1H), |
| | 7.42 (d, J = 9.7, 0.9 Hz, 1H), 7.20 (t, 1H), 7.06 (d, J = 7.6 Hz, 2H), 6.65 (s, |
| | 1H), 6.21 (s, 1H), 5.60 (dd, J = 11.4, 4.3 Hz, 1H), 5.48 (dd, J = 16.2, 1.7 Hz, |
| | 1H), 4.39-4.30 (m, 1H), 4.16 (dd, J = 16.3, 1.7 Hz, 1H), 3.85 (t, J = 11.6 Hz, |
| | 1H), 3.07 (hept, 1H), 2.01 (s, 6H), 1.81 (d, J = 14.9, 2.0 Hz, 1H), 1.46 (dd, J = |
| | 15.0, 10.2 Hz, 1H), 1.34 (dd, J = 6.9, 1.8 Hz, 6H), 0.56 (s, 3H), 0.44-0.38 (m, |
| | 1H), 0.30-0.24 (m, 1H), 0.17-0.11 (m, 1H), 0.07-0.02 (m, 1H). |
| I-79 | ¹H NMR (400 MHz, CDCl₃) δ 9.16 (t, J = 1.8 Hz, 1H), 8.09-8.03 (m, 1H), |
| | 8.00 (d, J = 7.8 Hz, 1H), 7.87 (dt, J = 7.7, 1.3 Hz, 1H), 7.65 (t, J = 7.8 Hz, |
| | 1H), 7.22 (t, J = 7.6 Hz, 1H), 7.05 (d, J = 7.6 Hz, 2H), 6.18 (s, 1H), 5.62- |
| | 5.48 (m, 2H), 4.44-4.35 (m, 1H), 4.28 (d, J = 17.3 Hz, 1H), 4.04 (s, 3H), 3.75 |
| | (t, J = 11.3 Hz, 1H), 3.44-3.34 (m, 1H), 2.01 (s, 6H), 1.83 (d, J = 14.8 Hz, |
| | 1H), 1.57 (dd, J = 12.7, 6.7 Hz, 6H), 1.47-1.38 (m, 1H), 0.58 (s, 3H), 0.43- |
| | 0.36 (m, 1H), 0.34-0.27 (m, 1H), 0.20-0.13 (m, 1H), 0.09-0.05 (m, 1H). |
| I-80 | ¹H NMR (400 MHz, CDCl₃) δ 9.21 (t, J = 1.8 Hz, 1H), 8.02-7.97 (m, 1H), |
| | 7.90 (dt, J = 7.6, 1.3 Hz, 1H), 7.64 (t, J = 7.8 Hz, 1H), 7.47 (d, J = 10.4 Hz, |
| | 1H), 7.20 (t, J = 7.6 Hz, 1H), 7.05 (d, J = 7.6 Hz, 2H), 6.20 (s, 1H), 6.15 (d, J = |
| | 0.8 Hz, 1H), 5.58 (dd, J = 11.3, 4.3 Hz, 1H), 5.52 (dd, J = 16.8, 1.7 Hz, |
| | 1H), 4.46-4.38 (m, 1H), 4.25 (dd, J = 16.8, 1.7 Hz, 1H), 3.90 (s, 3H), 3.78 (t, |
| | J = 11.6 Hz, 1H), 3.13-3.03 (m, 1H), 2.00 (s, 6H), 1.79 (d, J = 14.4 Hz, 1H), |
| | 1.46 (dd, J = 15.1, 10.2 Hz, 1H), 1.34 (d, J = 6.8 Hz, 3H), 1.31 (d, J = 6.8 Hz, |
| | 3H), 0.55 (s, 3H), 0.46-0.39 (m, 1H), 0.32-0.24 (m, 1H), 0.18-0.10 (m, |
| | 1H), 0.07-0.01 (m, 1H). |
| I-81 | ¹H NMR (400 MHz, CDCl₃) δ 9.15 (t, J = 1.8 Hz, 1H), 8.01 (d, J = 7.9 Hz, |
| | 1H), 7.88 (dt, J = 7.7, 1.3 Hz, 1H), 7.65 (t, J = 7.8 Hz, 1H), 7.48 (d, J = 10.3 |
| | Hz, 1H), 7.19 (t, J = 7.6 Hz, 1H), 7.03 (d, J = 7.6 Hz, 2H), 6.18-6.12 (m, |
| | 2H), 5.61-5.53 (m, 2H), 4.39-4.31 (m, 1H), 4.17-4.08 (m, 1H), 3.89 (s, |
| | 3H), 3.77 (t, J = 11.4 Hz, 1H), 3.13-3.03 (m, 1H), 2.00 (s, 6H), 1.94-1.85 |
| | (m, 1H), 1.61-1.38 (m, 7H), 1.34 (d, J = 6.8 Hz, 3H), 1.31 (d, J = 6.8 Hz, |
| | 4H), 1.15-1.05 (m, 1H), 0.59-0.47 (m, 1H). |
| I-82 | ¹H NMR (400 MHz, Chloroform-d) δ 9.05 (t, J = 1.8 Hz, 1H), 8.49 (s, 1H), |
| | 8.10 (dt, J = 8.2, 1.3 Hz, 1H), 7.87 (dt, J = 7.7, 1.4 Hz, 1H), 7.65 (t, J = 7.8 |
| | Hz, 1H), 7.28-7.24 (m, 1H), 7.12 (d, J = 7.7 Hz, 1H), 7.06 (d, J = 7.5 Hz, |
| | 1H), 6.60 (s, 1H), 5.63 (dd, J = 10.9, 4.1 Hz, 1H), 5.43 (d, J = 15.8 Hz, 1H), |
| | 4.44 (d, J = 15.8 Hz, 1H), 4.31-4.17 (m, 1H), 4.10 (t, J = 11.2 Hz, 1H), 3.95 |
| | (s, 3H), 3.24-3.13 (m, 1H), 2.25-2.13 (m, 2H), 1.84-1.75 (m, 5H), 1.72 (s, |
| | 3H), 1.62 (d, J = 14.9 Hz, 1H), 1.39 (dd, J = 10.7, 6.8 Hz, 6H), 0.81 (dd, J = |
| | 6.6, 4.3 Hz, 6H), 0.65 (s, 9H). |
| I-83 | ¹H NMR (400 MHz, CDCl₃) δ 9.07 (t, J = 1.9 Hz, 1H), 7.86 (dt, J = 7.3, 1.5 |
| | Hz, 1H), 7.57-7.45 (m, 3H), 7.26-7.23 (m, 1H), 7.09 (d, J = 7.6 Hz, 2H), |
| | 6.20 (s, 1H), 6.16-6.13 (m, 1H), 5.62 (dd, J = 16.8, 1.7 Hz, 1H), 5.53 (dd, J = |
| | 11.5, 4.5 Hz, 1H), 4.61-4.54 (m, 1H), 4.42-4.34 (m, 1H), 4.01 (t, J = 11.7 |
| | Hz, 1H), 3.89-3.82 (m, 4H), 3.63-3.55 (m, 1H), 3.54-3.48 (m, 1H), 3.13- |
| | 3.02 (m, 1H), 2.24-2.10 (m, 2H), 2.07 (s, 6H), 1.94-1.82 (m, 2H), 1.75- |
| | 1.65 (m, 1H), 1.52-1.44 (m, 1H), 1.34 (d, J = 6.8 Hz, 3H), 1.31 (d, J = 6.8 |
| | Hz, 3H). |
| I-84 | ¹H NMR (400 MHz, CDCl₃) δ 10.17-10.01 (m, 1H), 9.05 (t, J = 1.8 Hz, 1H), |
| | 7.84 (dt, J = 7.6, 1.4 Hz, 1H), 7.51 (t, J = 7.7 Hz, 1H), 7.46 (d, J = 10.3 Hz, |
| | 1H), 7.39-7.33 (m, 1H), 7.26 (t, J = 7.6 Hz, 1H), 7.08 (d, J = 7.6 Hz, 2H), |
| | 6.19 (s, 1H), 6.14 (s, 1H), 5.61 (dd, J = 16.7, 1.7 Hz, 1H), 5.52 (dd, J = 11.5, |
| | 4.5 Hz, 1H), 4.61-4.53 (m, 1H), 4.41-4.32 (m, 1H), 4.00 (t, J = 11.7 Hz, |
| | 1H), 3.90-3.81 (m, 4H), 3.61-3.55 (m, 1H), 3.51 (dd, J = 10.7, 3.2 Hz, 1H), |
| | 3.12-3.02 (m, 1H), 2.24-2.09 (m, 2H), 2.07 (s, 6H), 1.94-1.81 (m, 2H), |
| | 1.74-1.64 (m, 1H), 1.53-1.43 (m, 1H), 1.33 (d, J = 6.8 Hz, 3H), 1.30 (d, J = |
| | 6.8 Hz, 3H). |
| I-85 | ¹H NMR (400 MHz, Chloroform-d) δ 9.08 (s, 1H), 8.71 (s, 1H), 8.61-8.54 |
| | (m, 1H), 8.17 (d, J = 7.8 Hz, 1H), 7.88 (dt, J = 7.9, 1.3 Hz, 1H), 7.68 (t, J = |
| | 7.8 Hz, 1H), 7.20 (t, J = 7.6 Hz, 1H), 7.05 (d, J = 7.6 Hz, 2H), 6.24 (s, 1H), |
| | 5.33-5.20 (m, 2H), 4.49 (t, J = 11.6 Hz, 1H), 4.28 (d, J = 14.4 Hz, 1H), 4.14- |
| | 4.05 (m, 1H), 3.55-3.44 (m, 1H), 2.44 (s, 1H), 2.04 (s, 6H), 1.84 (dd, J = |
| | 15.7, 3.3 Hz, 1H), 1.60-1.50 (m, 13H). |
| I-86 | ¹H NMR (400 MHz, Chloroform-d) δ 8.75 (t, J = 1.8 Hz, 1H), 8.32 (s, 1H), |
| | 8.06 (dt, J = 8.0, 1.4 Hz, 1H), 7.91 (dt, J = 7.7, 1.4 Hz, 1H), 7.66 (t, J = 7.8 |
| | Hz, 1H), 7.25-7.19 (m, 1H), 7.08 (d, J = 7.6 Hz, 2H), 6.28 (s, 1H), 5.46 (dd, |
| | J = 11.2, 4.1 Hz, 1H), 5.23 (d, J = 15.0 Hz, 1H), 4.28-4.18 (m, 2H), 4.15- |
| | 4.04 (m, 1H), 2.56 (s, 3H), 2.43 (s, 1H), 2.08 (s, 6H), 2.03-1.95 (m, 1H), 1.86 |
| | -1.76 (m, 2H), 1.56-1.48 (m, 6H), 1.26-1.18 (m, 2H), 1.08-0.99 (m, 2H). |
| I-89 | ¹H NMR (400 MHz, CDCl₃) δ 10.44-10.12 (m, 1H), 9.05 (t, J = 1.8 Hz, 1H), |
| | 8.11 (d, J = 7.9 Hz, 1H), 7.86 (dt, J = 7.6, 1.4 Hz, 1H), 7.65 (t, J = 7.8 Hz, |
| | 1H), 7.42 (dd, J = 9.6, 0.9 Hz, 1H), 7.18 (t, J = 7.6 Hz, 1H), 6.99 (d, J = 7.6 |
| | Hz, 2H), 6.66-6.62 (m, 1H), 6.09 (s, 1H), 5.64-5.50 (m, 2H), 4.25-4.17 (m, |
| | 1H), 4.01-3.91 (m, 1H), 3.71 (t, J = 11.4 Hz, 1H), 3.14-3.00 (m, 1H), 2.46 |
| | (s, 1H), 2.00-1.92 (m, 6H), 1.78 (dd, J = 15.5, 3.3 Hz, 1H), 1.67-1.59 (m, |
| | 7H), 1.35 (d, J = 1.7 Hz, 3H), 1.33 (d, J = 1.7 Hz, 3H). |
| I-90 | ¹H NMR (400 MHz, CDCl₃) δ 8.88 (t, J = 1.7 Hz, 1H), 8.68 (s, 1H), 7.83 (dt, |
| | J = 7.5, 1.4 Hz, 1H), 7.69 (d, J = 7.9 Hz, 1H), 7.59 (t, J = 7.7 Hz, 1H), 7.28 (t, |
| | J = 7.6 Hz, 1H), 7.10 (d, J = 7.7 Hz, 2H), 6.25 (s, 1H), 5.59-5.42 (m, 2H), |
| | 4.81 (d, J = 16.3 Hz, 1H), 4.43-4.25 (m, 2H), 4.01 (s, 3H), 3.65 (d, J = 4.2 |
| | Hz, 2H), 3.55 (h, J = 6.1 Hz, 1H), 3.45 (hept, J = 6.9 Hz, 1H), 2.11 (s, 6H), |
| | 1.60 (d, J = 6.9 Hz, 3H), 1.57 (d, J = 6.8 Hz, 3H), 1.16 (d, J = 6.1 Hz, 3H), |
| | 1.13 (d, J = 6.0 Hz, 3H). |
| I-91 | ¹H NMR (400 MHz, CDCl₃) δ 10.05 (s, 1H), 8.65 (s, 1H), 8.53 (t, J = 1.8 Hz, |
| | 1H), 7.90 (d, J = 7.9 Hz, 1H), 7.80 (dt, J = 7.7, 1.3 Hz, 1H), 7.61 (t, J = 7.8 |
| | Hz, 1H), 7.22 (t, J = 7.6 Hz, 1H), 7.05 (d, J = 7.6 Hz, 2H), 6.21 (s, 1H), 5.30 |
| | (d, J = 14.9 Hz, 1H), 5.25 (dd, J = 11.2, 4.5 Hz, 1H), 4.73-4.63 (m, 2H), 4.41- |
| | 4.30 (m, 1H), 3.70-3.58 (m, 2H), 3.51 (hept, J = 7.0 Hz, 1H), 3.42 (hept, J = |
| | 6.1 Hz, 1H), 2.03 (s, 6H), 1.54 (d, J = 6.8 Hz, 3H), 1.52 (d, J = 6.8 Hz, 3H), |
| | 1.09 (d, J = 6.0 Hz, 3H), 1.01 (d, J = 6.1 Hz, 3H). |
| I-93 | ¹H NMR (400 MHz, Chloroform-d) δ 8.73 (s, 1H), 8.33 (s, 1H), 7.84 (dt, J = |
| | 7.6, 1.4 Hz, 1H), 7.68 (s, 1H), 7.57 (t, J = 7.7 Hz, 1H), 7.23 (d, J = 7.6 Hz, |
| | 1H), 7.10 (s, 1H), 7.08 (s, 1H), 6.25 (s, 1H), 5.43 (dd, J = 11.0, 4.1 Hz, 1H), |
| | 5.38 (d, J = 15.3 Hz, 1H), 4.59 (d, J = 15.3 Hz, 1H), 4.45 (t, J = 11.4 Hz, 1H), |
| | 4.40-4.32 (m, 1H), 3.79 (p, J = 8.2, 6.9 Hz, 1H), 3.59 (dd, J = 10.7, 7.6 Hz, |
| | 1H), 3.50 (dd, J = 10.7, 3.0 Hz, 1H), 3.27 (p, J = 6.9 Hz, 1H), 2.30 (s, 3H), |
| | 2.18-2.13 (m, 1H), 2.08 (s, 6H), 1.88-1.77 (m, 2H), 1.71-1.65 (m, 1H), |
| | 1.51-1.42 (m, 2H), 1.37 (dd, J = 6.9, 4.3 Hz, 6H). |
| I-95 | ¹H NMR (400 MHz, Chloroform-d) δ 8.96 (s, 1H), 7.81 (d, J = 7.4, 1.4 Hz, |
| | 1H), 7.59 (s, 1H), 7.53 (t, J = 7.6 Hz, 1H), 7.42 (dd, J = 9.6, 1.0 Hz, 1H), 7.24 |
| | (t, J = 7.5 Hz, 1H), 7.07 (d, J = 7.6 Hz, 2H), 6.62 (t, J = 1.0 Hz, 1H), 6.18 (s, |
| | 1H), 5.58 (dd, J = 16.2, 1.8 Hz, 1H), 5.52 (dd, J = 11.4, 4.4 Hz, 1H), 4.54 (dd, |
| | J = 16.2, 1.7 Hz, 1H), 4.34-4.25 (m, 1H), 4.09 (t, J = 11.6 Hz, 1H), 3.84 (p, |
| | 1H), 3.62-3.54 (m, 1H), 3.54-3.46 (m, 1H), 3.07 (hept, J = 6.9, 1.1 Hz, 1H), |
| | 2.22-2.08 (m, 2H), 2.05 (s, 6H), 1.92-1.80 (m, 2H), 1.72-1.65 (m, 1H), |
| | 1.51-1.43 (m, 1H), 1.34 (dd, J = 6.9, 1.5 Hz, 6H). |
| I-97 | ¹H NMR (400 MHz, Chloroform-d) δ 8.91 (s, 1H), 8.73 (t, J = 1.8 Hz, 1H), |
| | 8.34 (s, 1H), 8.13 (d, J = 8.0 Hz, 1H), 7.89 (dt, J = 7.7, 1.3 Hz, 1H), 7.67 (t, J = |
| | 7.8 Hz, 1H), 7.23-7.16 (m, 1H), 7.05 (d, J = 7.6 Hz, 2H), 6.64 (s, 1H), 6.21 |
| | (s, 1H), 5.39 (dd, J = 11.4, 4.0 Hz, 1H), 5.31 (d, J = 15.0 Hz, 1H), 4.26-4.16 |
| | (m, 2H), 4.10-3.99 (m, 1H), 2.44 (s, 1H), 2.06-1.95 (m, 7H), 1.80 (dd, J = |
| | 15.5, 3.2 Hz, 1H), 1.58-1.54 (m, 6H), 1.54 (s, 3H), 1.42-1.37 (m, 2H), 0.98- |
| | 0.88 (m, 2H). |
| I-100 | ¹H NMR (400 MHz, CDCl₃) δ 8.86 (t, J = 1.7 Hz, 1H), 8.57 (s, 1H), 7.81 (dt, |
| | J = 7.5, 1.4 Hz, 1H), 7.71 (d, J = 7.9 Hz, 1H), 7.59 (t, J = 7.7 Hz, 1H), 7.25 (t, |
| | J = 7.6 Hz, 1H), 7.10 (d, J = 7.6 Hz, 2H), 6.61 (s, 1H), 6.30 (s, 1H), 5.59- |
| | 5.47 (m, 1H), 5.43 (d, J = 15.6 Hz, 1H), 4.40 (d, J = 15.7 Hz, 1H), 4.24-4.08 |
| | (m, 2H), 3.94 (s, 3H), 3.19 (hept, J = 6.8 Hz, 1H), 2.06 (s, 6H), 2.04-1.98 (m, |
| | 1H), 1.56-1.45 (m, 1H), 1.41 (d, J = 6.9 Hz, 3H), 1.39 (d, J = 6.8 Hz, 3H), |
| | 1.31-1.24 (m, 1H), 0.75 (dt, J = 9.0, 4.5 Hz, 1H), 0.66 (dt, J = 8.9, 4.5 Hz, |
| | 1H), 0.57 (dt, J = 8.9, 4.4 Hz, 1H), 0.44 (dt, J = 9.0, 4.7 Hz, 1H), 0.37 (dt, J = |
| | 9.0, 4.5 Hz, 1H), 0.31 (dt, J = 9.1, 4.7 Hz, 1H), −0.04 (dt, J = 9.0, 4.7 Hz, |
| | 1H), −0.53 (dt, J = 9.1, 4.6 Hz, 1H). |
| I-101 | ¹H NMR (400 MHz, CDCl₃) δ 8.71-8.66 (m, 1H), 8.43 (s, 1H), 7.98 (d, J = |
| | 7.9 Hz, 1H), 7.83 (dt, J = 7.8, 1.3 Hz, 1H), 7.64 (t, J = 7.8 Hz, 1H), 7.22 (t, J = |
| | 7.6 Hz, 1H), 7.08 (d, J = 7.6 Hz, 2H), 6.62 (d, J = 1.0 Hz, 1H), 6.30 (s, 1H), |
| | 5.35 (dd, J = 11.4, 4.4 Hz, 1H), 5.30 (d, J = 14.8 Hz, 1H), 4.39-4.28 (m, 2H), |
| | 4.21-4.10 (m, 1H), 3.22-3.10 (m, 1H), 2.16-2.08 (m, 1H), 2.04 (s, 6H), |
| | 1.50-1.43 (m, 1H), 1.40 (d, J = 6.9 Hz, 6H), 1.23-1.19 (m, 1H), 0.69 (dt, J = |
| | 8.8, 4.3 Hz, 1H), 0.62 (dt, J = 8.7, 4.2 Hz, 1H), 0.54 (dt, J = 8.5, 4.2 Hz, |
| | 1H), 0.41 (dt, J = 9.1, 4.8 Hz, 1H), 0.34 (dt, J = 8.9, 4.5 Hz, 1H), 0.29 (dt, J = |
| | 9.2, 4.7 Hz, 1H), −0.08 (dt, J = 9.1, 4.6 Hz, 1H), −0.58 (dt, J = 9.1, 4.7 Hz, |
| | 1H). |
| I-102 | ¹H NMR (400 MHz, CDCl₃) δ 8.66 (t, J = 1.8 Hz, 1H), 8.37 (s, 1H), 7.85- |
| | 7.73 (m, 2H), 7.59 (t, J = 7.8 Hz, 1H), 7.23 (t, J = 7.6 Hz, 1H), 7.08 (d, J = |
| | 7.6 Hz, 2H), 6.61 (s, 1H), 6.29 (s, 1H), 5.32 (dd, J = 11.4, 4.4 Hz, 1H), 5.28 |
| | (d, J = 14.8 Hz, 1H), 4.38-4.26 (m, 2H), 4.15 (tt, J = 11.2, 3.7 Hz, 1H), 2.17- |
| | 2.08 (m, 2H), 2.04 (s, 6H), 1.45 (ddd, J = 14.3, 10.0, 3.2 Hz, 1H), 1.23-1.18 |
| | (m, 1H), 1.18-1.14 (m, 3H), 1.14-1.11 (m, 1H), 0.71-0.65 (m, 1H), 0.61 |
| | (dt, J = 8.7, 4.3 Hz, 1H), 0.53 (dt, J = 8.6, 4.2 Hz, 1H), 0.41 (dt, J = 9.1, 4.7 |
| | Hz, 1H), 0.33 (dt, J = 8.9, 4.5 Hz, 1H), 0.28 (dt, J = 9.1, 4.6 Hz, 1H), −0.08 |
| | (dt, J = 9.0, 4.6 Hz, 1H), −0.58 (dt, J = 9.1, 4.7 Hz, 1H). |
| I-103 | ¹H NMR (400 MHz, CDCl₃) δ 8.64 (s, 1H), 8.37 (s, 1H), 8.11 (d, J = 7.9 Hz, |
| | 1H), 7.85 (dt, J = 7.7, 1.4 Hz, 1H), 7.66 (t, J = 7.8 Hz, 1H), 7.21 (t, J = 7.7 |
| | Hz, 1H), 7.05 (d, J = 7.7 Hz, 2H), 6.63 (s, 1H), 6.21 (s, 1H), 5.45-5.36 (m, |
| | 2H), 4.33 (t, J = 11.5 Hz, 1H), 4.25 (d, J = 14.9 Hz, 1H), 4.22-4.13 (m, 1H), |
| | 2.18-2.09 (m, 1H), 2.03 (s, 6H), 1.93 (ddd, J = 14.4, 10.3, 4.9 Hz, 1H), 1.35- |
| | 1.27 (m, 1H), 1.20-1.15 (m, 3H), 1.15-1.12 (m, 1H), 0.58-0.42 (m, 2H), |
| | 0.33-0.24 (m, 1H), 0.10 (dt, J = 9.3, 4.8 Hz, 1H), −0.33-−0.43 (m, 1H). |
| I-105 | ¹H NMR (400 MHz, CDCl₃) δ 8.79 (t, J = 1.8 Hz, 1H), 8.40 (s, 1H), 7.88 (d, J = |
| | 7.9 Hz, 1H), 7.82 (dt, J = 7.8, 1.3 Hz, 1H), 7.58 (t, J = 7.8 Hz, 1H), 7.22 (t, |
| | J = 7.6 Hz, 1H), 7.07 (d, J = 7.6 Hz, 2H), 6.62 (d, J = 1.1 Hz, 1H), 6.26 (s, |
| | 1H), 5.46 (dd, J = 11.3, 4.3 Hz, 1H), 5.24 (d, J = 14.8 Hz, 1H), 4.51-4.43 (m, |
| | 1H), 4.38 (d, J = 14.9 Hz, 1H), 4.30 (t, J = 11.6 Hz, 1H), 3.22-3.10 (m, 1H), |
| | 2.01 (s, 6H), 1.88 (dd, J = 15.2, 8.4 Hz, 1H), 1.74-1.68 (m, 1H), 1.42-1.40 |
| | (m, 3H), 1.40-1.38 (m, 3H), 0.66 (s, 3H), 0.41 (s, 3H), 0.10-0.03 (m, 1H), −0.12-−0.22 |
| | (m, 2H), −0.42-−0.54 (m, 2H). |
| I-106 | ¹H NMR (400 MHz, CDCl₃) δ 8.86 (t, J = 1.8 Hz, 1H), 8.50 (s, 1H), 7.81 (dt, |
| | J = 7.6, 1.4 Hz, 1H), 7.77-7.69 (m, 1H), 7.59 (t, J = 7.7 Hz, 1H), 7.24 (t, J = |
| | 7.6 Hz, 1H), 7.06 (d, J = 7.6 Hz, 2H), 6.54 (s, 1H), 6.20 (s, 1H), 5.55-5.44 |
| | (m, 2H), 4.33 (d, J = 15.7 Hz, 1H), 4.15-4.02 (m, 2H), 3.92 (s, 3H), 3.17 |
| | (hept, J = 6.8 Hz, 1H), 2.04 (s, 6H), 1.92-1.83 (m, 2H), 1.68-1.53 (m, 1H), |
| | 1.40 (d, J = 6.8 Hz, 3H), 1.38 (d, J = 6.8 Hz, 3H), 1.34 (d, J = 2.7 Hz, 3H), |
| | 1.31-1.24 (m, 4H). |
| I-107 | ¹H NMR (400 MHz, CDCl₃) δ 8.62 (t, 1H), 8.36 (s, 1H), 8.01-7.95 (m, 1H), |
| | 7.80 (dt, J = 7.7, 1.3 Hz, 1H), 7.63 (t, J = 7.8 Hz, 1H), 7.20 (t, J = 7.6 Hz, |
| | 1H), 7.02 (d, J = 7.6 Hz, 2H), 6.61 (s, 1H), 6.17 (s, 1H), 5.40-5.30 (m, 2H), |
| | 4.32-4.21 (m, 2H), 4.12-4.01 (m, 1H), 2.18-2.08 (m, 1H), 2.01 (s, 6H), |
| | 1.92-1.77 (m, 2H), 1.35-1.29 (m, 4H), 1.29-1.23 (m, 4H), 1.20-1.11 (m, |
| | 4H). |
| I-109 | ¹H NMR (400 MHz, CDCl₃) δ 8.63 (t, J = 1.8 Hz, 1H), 8.36 (s, 1H), 7.80 (dt, |
| | J = 7.5, 1.5 Hz, 1H), 7.69-7.63 (m, 1H), 7.55 (t, J = 7.8 Hz, 1H), 7.28-7.21 |
| | (m, 1H), 7.06 (d, J = 7.6 Hz, 2H), 6.62 (d, J = 1.1 Hz, 1H), 6.21 (s, 1H), 5.38 |
| | (dd, J = 11.3, 4.4 Hz, 1H), 5.31 (d, J = 15.1 Hz, 1H), 4.52 (d, J = 15.1 Hz, |
| | 1H), 4.44 (t, J = 11.6 Hz, 1H), 4.37-4.26 (m, 1H), 3.79-3.71 (m, 1H), 3.67 |
| | (dd, J = 10.8, 3.1 Hz, 1H), 3.22-3.10 (m, 2H), 2.04 (s, 6H), 1.40 (d, J = 6.9 |
| | Hz, 6H), 0.53-0.39 (m, 4H). |
| I-110 | ¹H NMR (400 MHz, Chloroform-d) δ 8.80 (t, J = 1.8 Hz, 1H), 8.41 (s, 1H), |
| | 8.09 (ddd, J = 7.9, 1.9, 1.1 Hz, 1H), 8.01-7.69 (m, 2H), 7.64 (t, J = 7.8 Hz, |
| | 1H), 7.21 (t, J = 7.6 Hz, 1H), 7.11 (d, J = 7.6 Hz, 1H), 7.06 (d, J = 7.5 Hz, |
| | 1H), 6.63 (d, J = 1.0 Hz, 1H), 5.46 (dd, J = 11.2, 4.3 Hz, 1H), 5.30-5.24 (m, |
| | 1H), 4.42-4.32 (m, 2H), 4.29-4.19 (m, 1H), 3.22-3.09 (m, 1H), 2.03 (s, |
| | 3H), 1.88-1.78 (m, 4H), 1.75 (s, 3H), 1.60 (dd, J = 15.1, 1.6 Hz, 1H), 1.40 (d, |
| | J = 6.9 Hz, 6H), 0.61 (s, 9H). |
| I-111 | ¹H NMR (400 MHz, CDCl₃) δ 8.82 (s, 1H), 8.69 (s, 1H), 8.30 (s, 1H), 8.12 (d, |
| | J = 8.1 Hz, 1H), 7.86 (d, J = 7.8 Hz, 1H), 7.65 (t, J = 7.8 Hz, 1H), 7.24-7.17 |
| | (m, 1H), 7.07 (d, J = 7.6 Hz, 2H), 6.25 (s, 1H), 5.36 (dd, J = 11.5, 4.4 Hz, |
| | 1H), 5.30 (d, J = 14.4 Hz, 1H), 4.86 (spt, J = 6.6 Hz, 1H), 4.49 (t, J = 11.6 |
| | Hz, 1H), 4.37 (d, J = 14.4 Hz, 1H), 4.31-4.22 (m, 1H), 2.01 (s, 6H), 1.84 (dd, |
| | J = 15.4, 8.8 Hz, 1H), 1.72 (d, J = 6.8 Hz, 6H), 1.67-1.54 (m, 1H, overlapped |
| | with water), 0.63 (s, 9H). 1 missing labile proton. |
| I-112 | ¹H NMR (400 MHz, Chloroform-d) δ 8.71 (t, J = 1.8 Hz, 1H), 8.43 (s, 1H), |
| | 8.11 (dd, J = 8.0, 1.7 Hz, 1H), 7.88 (dt, J = 7.7, 1.4 Hz, 1H), 7.66 (t, J = 7.8 |
| | Hz, 1H), 7.23 (d, J = 7.6 Hz, 1H), 7.09 (d, J = 7.6 Hz, 2H), 6.76 (s, 1H), 6.31 |
| | (s, 1H), 5.43 (d, J = 7.3 Hz, 1H), 5.28 (d, J = 14.8 Hz, 1H), 4.47-4.31 (m, |
| | 2H), 4.16 (d, J = 14.8 Hz, 1H), 3.85-3.75 (m, 2H), 2.06 (s, 6H), 1.81 (d, J = |
| | 15.0 Hz, 1H), 1.54-1.46 (m, 1H), 1.43 (d, J = 2.8 Hz, 6H), 0.48 (s, 3H), 0.41- |
| | 0.31 (m, 1H), 0.31-0.21 (m, 1H), 0.19-0.10 (m, 1H), 0.06-0.01 (m, 1H). |
| I-114 | ¹H NMR (400 MHz, CDCl₃) δ 8.61 (d, J = 2.9 Hz, 2H), 8.08 (dt, J = 8.0, 1.4 |
| | Hz, 1H), 7.86 (dt, J = 7.7, 1.4 Hz, 1H), 7.64 (t, J = 7.8 Hz, 1H), 7.20 (t, J = |
| | 7.6 Hz, 1H), 7.04 (d, J = 7.6 Hz, 2H), 6.20 (s, 1H), 5.33 (dd, J = 11.5, 4.1 Hz, |
| | 1H), 5.28 (d, J = 14.7 Hz, 1H), 4.39 (t, J = 11.6 Hz, 1H), 4.28 (d, J = 14.6 Hz, |
| | 1H), 4.07 (tt, J = 11.3, 3.7 Hz, 1H), 3.57 (hept, J = 6.9 Hz, 1H), 2.43 (s, 1H), |
| | 2.02 (s, 7H), 1.82 (dd, J = 15.7, 3.3 Hz, 1H), 1.58-1.51 (m, 6H), 1.45 (d, J = |
| | 1.6 Hz, 3H), 1.43 (d, J = 1.6 Hz, 3H). |
| I-115 | ¹H NMR (400 MHz, CDCl₃) δ 8.64 (t, J = 1.8 Hz, 1H), 8.30 (s, 1H), 7.80 (dt, |
| | J = 7.7, 1.4 Hz, 1H), 7.71-7.64 (m, 1H), 7.55 (t, J = 7.8 Hz, 1H), 7.27-7.21 |
| | (m, 1H), 7.06 (d, J = 7.6 Hz, 2H), 6.61 (s, 1H), 6.21 (s, 1H), 5.36 (dd, J = |
| | 11.2, 4.3 Hz, 1H), 5.29 (d, J = 15.2 Hz, 1H), 4.51 (d, J = 15.1 Hz, 1H), 4.41 |
| | (t, J = 11.5 Hz, 1H), 4.36-4.26 (m, 1H), 3.79-3.70 (m, 1H), 3.66 (dd, J = |
| | 10.8, 3.1 Hz, 1H), 3.21-3.11 (m, 1H), 2.18-2.08 (m, 1H), 2.04 (s, 6H), 1.18- |
| | 1.11 (m, 4H), 0.53-0.38 (m, 4H). |
| I-116 | ¹H NMR (400 MHz, CDCl₃) δ 8.74 (t, J = 1.8 Hz, 1H), 8.34 (s, 1H), 8.18 (s, |
| | 1H), 8.07 (d, J = 7.9 Hz, 1H), 7.82 (d, J = 7.6 Hz, 1H), 7.63 (t, J = 7.8 Hz, |
| | 1H), 7.21 (t, J = 7.6 Hz, 1H), 7.07 (d, J = 7.6 Hz, 2H), 6.63 (s, 1H), 6.28 (s, |
| | 1H), 5.39 (dd, J = 11.0, 3.9 Hz, 1H), 5.28 (d, J = 14.9 Hz, 1H), 4.34 (d, J = |
| | 14.9 Hz, 1H), 4.28 (t, J = 11.4 Hz, 1H), 4.22-4.13 (m, 1H), 2.17-2.08 (m, |
| | 1H), 2.02 (s, 6H), 1.98-1.91 (m, 1H), 1.88-1.74 (m, 2H), 1.73-1.62 (m, |
| | 2H), 1.39 (t, J = 7.7 Hz, 2H), 1.20-1.08 (m, 4H), 0.67 (s, 3H). (missing a |
| | cyclobutyl proton. see note) |
| I-118 | ¹H NMR (400 MHz, CDCl₃) δ 9.74 (s, 1H), 8.66 (t, J = 1.8 Hz, 1H), 8.30 (s, |
| | 1H), 7.81 (dt, J = 7.7, 1.4 Hz, 1H), 7.77 (d, J = 7.9 Hz, 1H), 7.58 (t, J = 7.8 |
| | Hz, 1H), 7.23 (t, J = 7.6 Hz, 1H), 7.06 (d, J = 7.6 Hz, 2H), 6.61 (s, 1H), 6.20 |
| | (s, 1H), 5.39-5.27 (m, 2H), 4.56 (d, J = 15.2 Hz, 1H), 4.43 (t, J = 11.6 Hz, |
| | 1H), 4.37-4.27 (m, 1H), 3.80-3.71 (m, 1H), 3.62 (dd, J = 10.7, 7.7 Hz, 1H), |
| | 3.52 (dd, J = 10.6, 3.1 Hz, 1H), 2.13 (tt, J = 8.1, 5.3 Hz, 1H), 2.04 (s, 6H), |
| | 1.63-1.57 (m, 4H), 1.56-1.42 (m, 4H), 1.20-1.09 (m, 4H). |
| I-119 | ¹H NMR (400 MHz, CDCl₃) δ 9.62 (s, 1H), 8.72 (t, J = 1.9 Hz, 1H), 8.31 (s, |
| | 1H), 7.81 (dt, J = 7.3, 1.5 Hz, 1H), 7.60 (d, J = 8.0 Hz, 1H), 7.54 (t, J = 7.7 |
| | Hz, 1H), 7.24 (t, J = 7.6 Hz, 1H), 7.08 (d, J = 7.6 Hz, 2H), 6.23 (s, 1H), 5.40 |
| | (dd, J = 11.1, 4.3 Hz, 1H), 5.36 (d, J = 15.3 Hz, 1H), 4.61 (d, J = 15.3 Hz, |
| | 1H), 4.44 (t, J = 11.5 Hz, 1H), 4.37-4.27 (m, 1H), 3.82-3.75 (m, 1H), 3.64 |
| | (dd, J = 10.7, 7.6 Hz, 1H), 3.55 (dd, J = 10.7, 3.1 Hz, 1H), 3.27 (hept, J = 7.0 |
| | Hz, 1H), 2.30 (s, 3H), 2.07 (s, 6H), 1.66-1.60 (m, 4H), 1.58-1.46 (m, 4H), |
| | 1.38 (d, J = 6.7 Hz, 3H), 1.36 (d, J = 6.7 Hz, 3H). |
| I-120 | ¹H NMR (400 MHz, CDCl₃) δ 8.66 (t, J = 1.8 Hz, 1H), 8.37 (s, 1H), 7.81 (dd, |
| | J = 7.3, 1.5 Hz, 2H), 7.58 (t, J = 7.8 Hz, 1H), 7.22 (t, J = 7.6 Hz, 1H), 7.06 (d, |
| | J = 7.6 Hz, 2H), 6.62 (d, J = 1.0 Hz, 1H), 6.21 (s, 1H), 5.42-5.27 (m, 2H), |
| | 4.58 (d, J = 15.1 Hz, 1H), 4.46 (t, J = 11.6 Hz, 1H), 4.39-4.27 (m, 1H), 3.83- |
| | 3.71 (m, 1H), 3.63 (dd, J = 10.6, 7.8 Hz, 1H), 3.53 (dd, J = 10.6, 3.1 Hz, |
| | 1H), 3.16 (dhept, J = 7.0, 0.9 Hz, 1H), 2.04 (s, 6H), 1.66-1.57 (m, 5H), 1.55- |
| | 1.46 (m, 3H), 1.40 (d, J = 6.9 Hz, 6H). |
| I-122 | ¹H NMR (400 MHz, Chloroform-d) δ 8.80 (t, J = 1.8 Hz, 1H), 8.41 (s, 1H), |
| | 8.10 (dt, J = 8.0, 1.4 Hz, 1H), 7.84 (dt, J = 7.7, 1.4 Hz, 1H), 7.63 (t, J = 7.8 |
| | Hz, 1H), 7.22 (t, J = 7.7 Hz, 1H), 7.03 (d, J = 7.5 Hz, 1H), 6.77 (d, J = 7.9 |
| | Hz, 1H), 6.62 (d, J = 1.0 Hz, 1H), 5.47 (dd, J = 11.1, 4.3 Hz, 1H), 5.26 (d, J = |
| | 14.8 Hz, 1H), 4.48-4.31 (m, 2H), 4.31-4.18 (m, 1H), 3.24-3.07 (m, 1H), |
| | 1.88-1.80 (m, 4H), 1.79 (s, 3H), 1.47-1.42 (m, 1H), 1.40 (d, J = 7.0 Hz, |
| | 6H), 0.91-0.68 (m, 4H), 0.61 (s, 9H), 0.57-0.51 (m, 1H) |
| I-124 | ¹H NMR (400 MHz, CDCl₃) δ 8.65 (t, J = 1.7 Hz, 1H), 8.42 (s, 1H), 7.92 |
| | (d, J = 7.8 Hz, 1H), 7.79 (d, J = 7.5 Hz, 1H), 7.59 (t, J = 7.8 Hz, 1H), 7.22 (t, J = |
| | 7.6 Hz, 1H), 7.07 (d, J = 7.6 Hz, 2H), 6.63 (d, J = 1.0 Hz, 1H), 6.25 (s, 1H), |
| | 5.40 (dd, J = 10.1, 3.0 Hz, 1H), 5.33 (d, J = 14.9 Hz, 1H), 4.49-4.38 (m, 1H), |
| | 4.38-4.30 (m, 1H), 4.26 (d, J = 14.9 Hz, 1H), 3.24-3.10 (m, 1H), 2.33-2.15 |
| | (m, 2H), 2.13-2.05 (m, 1H), 2.03 (s, 6H), 2.01-1.90 (m, 2H), 1.82-1.73 (m, |
| | 1H), 1.51-1.42 (m, 1H), 1.40 (d, J = 7.0 Hz, 6H), 1.38-1.28 (m, 1H). |
| I-125 | ¹H NMR (400 MHz, CDCl₃) δ 8.85 (s, 1H), 8.59 (s, 1H), 7.85 (d, J = 7.9 Hz, |
| | 1H), 7.80 (d, J = 7.6 Hz, 1H), 7.61 (t, J = 7.7 Hz, 1H), 7.24 (t, J = 7.6 Hz, |
| | 1H), 7.09 (d, J = 7.6 Hz, 2H), 6.86 (s, 1H), 6.25 (s, 1H), 5.63-5.52 (m, 1H), |
| | 5.52-5.36 (m, 1H), 4.70-4.36 (m, 2H), 4.33-4.08 (m, 1H), 4.02 (s, 3H), |
| | 3.24 (hept, J = 6.7 Hz, 1H), 2.49-2.13 (m, 3H), 2.07 (s, 6H), 2.06-1.92 (m, |
| | 3H), 1.88-1.77 (m, 1H), 1.57-1.47 (m, 1H), 1.43 (d, J = 6.8 Hz, 3H), 1.41 |
| | (d, J = 6.7 Hz, 3H). |
| I-127 | ¹H NMR (400 MHz, CDCl₃) δ 8.64-8.60 (m, 1H), 8.55-8.51 (m, 1H), 8.07- |
| | 8.01 (m, 1H), 7.78 (dt, J = 7.7, 1.4 Hz, 1H), 7.63 (t, J = 7.8 Hz, 1H), 7.21 (t, J = |
| | 7.6 Hz, 1H), 7.03 (d, J = 7.7 Hz, 2H), 6.20 (s, 1H), 5.32-5.25 (m, 2H), 4.48 |
| | (t, J = 11.5 Hz, 1H), 4.31 (d, J = 14.5 Hz, 1H), 4.13-4.03 (m, 1H), 3.57 |
| | (hept, J = 7.0 Hz, 1H), 2.04-1.97 (m, 6H), 1.92-1.70 (m, 2H), 1.45 (d, J = |
| | 1.4 Hz, 6H), 1.23-1.10 (m, 1H), 0.93-0.89 (m, 3H), 0.89-0.83 (m, 1H), |
| | 0.20-0.05 (m, 4H). |
| I-129 | ¹H NMR (400 MHz, CDCl₃) δ 8.79 (s, 1H), 8.54-8.51 (m, 1H), 8.33 (s, 1H), |
| | 8.24 (d, J = 7.9 Hz, 1H), 7.78 (dt, J = 7.6, 1.4 Hz, 1H), 7.67 (t, J = 7.8 Hz, |
| | 1H), 7.18 (t, J = 7.6 Hz, 1H), 6.98 (d, J = 7.7 Hz, 2H), 6.11 (s, 1H), 5.40 (d, J = |
| | 14.5 Hz, 1H), 5.25 (dd, J = 11.4, 4.2 Hz, 1H), 4.85 (h, J = 6.8 Hz, 1H), 4.44 |
| | (t, J = 11.5 Hz, 1H), 4.27 (d, J = 14.5 Hz, 1H), 4.11-3.98 (m, 1H), 2.00- |
| | 1.80 (m, 7H), 1.72 (d, J = 6.6 Hz, 7H), 1.23-1.11 (m, 1H), 0.94-0.82 (m, |
| | 4H), 0.22-0.15 (m, 2H), 0.15-0.10 (m, 2H). |
| I-130 | ¹H NMR (400 MHz, CDCl₃) δ 10.7 (br, 1H). 8.62 (t, J = 1.8 Hz, 1H), 8.37 (s, |
| | 1H), 8.12 (d, J = 8.0 Hz, 1H), 7.80 (dt, J = 7.7, 1.4 Hz, 1H), 7.65 (t, J = 7.8 |
| | Hz, 1H), 7.19 (t, J = 7.6 Hz, 1H), 6.99 (d, J = 7.6 Hz, 2H), 6.61 (s, 1H), 6.13 |
| | (s, 1H), 5.42-5.27 (m, 2H), 4.25 (d, J = 9.9 Hz, 1H), 4.25 (d, J = 13.2 Hz, |
| | 1H), 4.04 (tt, J = 11.3, 3.9 Hz, 1H), 2.13 (tt, J = 8.1, 5.3 Hz, 1H), 1.94 (s, 6H), |
| | 1.81-1.69 (m, 1H), 1.68-1.55 (m, 1H), 1.44-1.30 (m, 1H), 1.21-1.13 (m, |
| | 4H), 1.13-1.05 (m, 1H), 1.00-0.86 (m, 1H), 0.81 (d, J = 6.6 Hz, 3H), 0.79 |
| | (d, J = 6.5 Hz, 3H). |
| I-131 | ¹H NMR (400 MHz, CDCl₃) δ 8.99 (t, J = 1.8 Hz, 1H), 8.95 (br s, 1H), 8.45 |
| | (s, 1H), 8.01 (dt, J = 8.3, 1.3 Hz, 1H), 7.88 (dt, J = 7.7, 1.3 Hz, 1H), 7.63 (t, J = |
| | 7.8 Hz, 1H), 7.22 (t, J = 7.6 Hz, 1H), 7.07 (d, J = 7.6 Hz, 2H), 6.27 (d, J = |
| | 0.9 Hz, 1H), 6.23 (s, 1H), 5.54 (dd, J = 11.3, 4.3 Hz, 1H), 5.41 (d, J = 15.7 |
| | Hz, 1H), 4.49-4.32 (m, 1H), 4.23 (d, J = 15.7 Hz, 1H), 4.04 (t, J = 11.5 Hz, |
| | 1H), 4.00 (s, 3H), 2.02 (s, 6H), 2.00-1.94 (m, 1H), 1.83 (d, J = 14.9 Hz, 1H), |
| | 1.46 (dd, J = 15.1, 10.2 Hz, 1H), 1.18-1.06 (m, 2H), 0.95-0.78 (m, 2H), |
| | 0.53 (s, 3H), 0.39 (dt, J = 9.7, 5.0 Hz, 1H), 0.28 (dt, J = 9.8, 5.0 Hz, 1H), 0.15 |
| | (dt, J = 9.3, 4.8 Hz, 1H), 0.04 (dt, J = 9.5, 4.8 Hz, 1H). |
| I-132 | ¹H NMR (400 MHz, CDCl₃) δ 8.75 (s, 1H), 8.58 (t, J = 1.8 Hz, 1H), 8.30 (s, |
| | 1H), 7.93 (d, J = 7.9 Hz, 1H), 7.83 (dt, J = 7.7, 1.4 Hz, 1H), 7.61 (t, J = 7.8 |
| | Hz, 1H), 7.22 (t, J = 7.6 Hz, 1H), 7.04 (d, J = 7.6 Hz, 2H), 6.16 (s, 1H), 5.36 |
| | (d, J = 15.0 Hz, 1H), 5.28 (dd, J = 11.3, 4.5 Hz, 1H), 4.85 (hept, J = 6.7 Hz, |
| | 1H), 4.66 (d, J = 15.0 Hz, 1H), 4.58 (t, J = 11.6 Hz, 1H), 4.40-4.28 (m, 1H), |
| | 3.72-3.54 (m, 2H), 3.39 (hept, J = 6.0 Hz, 1H), 2.01 (s, 6H), 1.71 (d, J = 6.7 |
| | Hz, 6H), 1.06 (d, J = 6.1 Hz, 3H), 0.98 (d, J = 6.0 Hz, 3H). |
| I-133 | ¹H NMR (400 MHz, Chloroform-d) δ 8.72 (t, J = 1.8 Hz, 1H), 8.40 (s, 1H), |
| | 8.11 (dt, J = 8.2, 1.4 Hz, 1H), 7.89 (dt, J = 7.7, 1.4 Hz, 1H), 7.66 (t, J = 7.8 |
| | Hz, 1H), 7.23 (d, J = 7.6 Hz, 1H), 7.09 (d, J = 7.7 Hz, 2H), 6.65 (d, J = 0.8 |
| | Hz, 1H), 6.31 (s, 1H), 5.44 (dd, J = 10.2, 3.2 Hz, 1H), 5.28 (d, J = 14.9 Hz, |
| | 1H), 4.46-4.29 (m, 2H), 4.16 (d, J = 14.8 Hz, 1H), 3.80-3.67 (m, 1H), 2.48- |
| | 2.37 (m, 4H), 2.20-1.96 (m, 8H), 1.81 (d, J = 15.0 Hz, 1H), 1.49 (dd, J = |
| | 15.1, 9.3 Hz, 1H), 0.48 (s, 3H), 0.36 (dt, J = 9.7, 5.0 Hz, 1H), 0.30-0.21 (m, |
| | 1H), 0.15 (p, J = 4.9 Hz, 1H), 0.07-0.01 (m, 1H) plus 1H not observed. |
| I-135 | ¹H NMR (400 MHz, Chloroform-d) δ 8.81 (t, J = 1.9 Hz, 1H), 8.41 (s, 1H), |
| | 8.09 (dt, J = 8.1, 1.4 Hz, 1H), 7.85 (dt, J = 7.8, 1.4 Hz, 1H), 7.64 (t, J = 7.8 |
| | Hz, 1H), 7.31 (t, J = 7.6 Hz, 1H), 7.24 (d, J = 8.0 Hz, 1H), 7.05 (d, J = 7.4 |
| | Hz, 1H), 6.63 (d, J = 1.0 Hz, 1H), 5.48 (dd, J = 11.2, 4.3 Hz, 1H), 5.26 (d, J = |
| | 14.8 Hz, 1H), 4.42-4.32 (m, 2H), 4.28-4.18 (m, 1H), 3.24-3.09 (m, 1H), |
| | 2.49 (hept, J = 6.7 Hz, 1H), 1.80 (s, 4H), 1.76 (s, 3H), 1.60 (d, J = 15.1 Hz, |
| | 1H), 1.40 (d, J = 6.9 Hz, 6H), 1.15 (d, J = 6.8 Hz, 3H), 1.10 (d, J = 6.9 Hz, |
| | 3H), 0.61 (s, 9H). |
| I-136 | ¹H NMR (400 MHz, Chloroform-d) δ 8.79 (t, J = 1.9 Hz, 1H), 8.42 (s, 1H), |
| | 7.94 (d, J = 7.8 Hz, 1H), 7.85 (dt, J = 7.7, 1.3 Hz, 1H), 7.62 (t, J = 7.8 Hz, |
| | 1H), 7.33 (t, J = 7.7 Hz, 1H), 7.19 (d, J = 7.8 Hz, 1H), 7.13 (d, J = 7.5 Hz, |
| | 1H), 6.63 (d, J = 1.1 Hz, 1H), 5.44 (dd, J = 11.2, 4.3 Hz, 1H), 5.25 (d, J = |
| | 14.8 Hz, 1H), 4.44-4.32 (m, 2H), 4.32-4.23 (m, 1H), 3.24-3.08 (m, 1H), |
| | 2.19 (hept, J = 6.5 Hz, 1H), 2.04 (s, 3H), 1.82 (dd, J = 15.2, 8.4 Hz, 1H), 1.75 |
| | (s, 3H), 1.58 (d, J = 15.0 Hz, 1H), 1.40 (d, J = 6.9 Hz, 6H), 0.98 (d, J = 6.9 |
| | Hz, 6H), 0.59 (s, 9H). |
| I-137 | ¹H NMR (400 MHz, Chloroform-d) δ 8.80 (t, J = 1.8 Hz, 1H), 8.35 (s, 1H), |
| | 8.09 (dt, J = 8.0, 1.4 Hz, 1H), 7.84 (dt, J = 7.7, 1.4 Hz, 1H), 7.63 (t, J = 7.8 |
| | Hz, 1H), 7.26-7.22 (m, 1H), 7.12 (d, J = 7.7 Hz, 1H), 7.06 (d, J = 7.5 Hz, |
| | 1H), 6.63 (s, 1H), 5.47 (dd, J = 11.2, 4.3 Hz, 1H), 5.25 (d, J = 14.8 Hz, 1H), |
| | 4.40-4.28 (m, 2H), 4.26-4.10 (m, 1H), 2.27-2.06 (m, 3H), 1.88-1.76 (m, |
| | 5H), 1.74 (s, 3H), 1.58 (d, J = 15.2 Hz, 1H), 1.19-1.08 (m, 4H), 0.85-0.75 |
| | (m, 6H), 0.60 (s, 9H) |
| I-138 | ¹H NMR (400 MHz, CDCl₃) δ 9.43 (s, 1H), 8.68 (t, J = 1.8 Hz, 1H), 8.37 (s, |
| | 1H), 7.88-7.79 (m, 2H), 7.60 (t, J = 7.8 Hz, 1H), 7.23 (t, J = 7.6 Hz, 1H), |
| | 7.06 (d, J = 7.6 Hz, 2H), 6.65 (d, J = 0.8 Hz, 1H), 6.22 (s, 1H), 5.42-5.30 (m, |
| | 2H), 4.58 (d, J = 15.2 Hz, 1H), 4.47 (t, J = 11.6 Hz, 1H), 4.38-4.28 (m, 1H), |
| | 3.80-3.62 (m, 2H), 3.62-3.55 (m, 1H), 3.53-3.39 (m, 1H), 2.47-2.37 (m, |
| | 4H), 2.19-2.05 (m, 2H), 2.05 (s, 6H), 1.11 (d, J = 6.1 Hz, 3H), 1.06 (d, J = |
| | 6.1 Hz, 3H). |
| I-139 | ¹H NMR (400 MHz, CDCl₃) δ 8.89-8.86 (m, 1H), 8.53 (s, 1H), 7.84-7.80 |
| | (m, 1H), 7.57-7.47 (m, 2H), 7.27 (t, J = 7.6 Hz, 1H), 7.09 (d, J = 7.7 Hz, |
| | 2H), 6.22 (s, 1H), 5.53-5.42 (m, 2H), 4.70 (d, J = 16.1 Hz, 1H), 4.36-4.20 |
| | (m, 2H), 4.01 (s, 3H), 3.63 (d, J = 4.5 Hz, 2H), 3.53 (hept, J = 6.1 Hz, 1H), |
| | 2.15-2.04 (m, 7H), 1.62-1.47 (m, 2H), 1.29 (dd, J = 8.2, 3.4 Hz, 2H), 1.16 |
| | (d, J = 6.0 Hz, 3H), 1.12 (d, J = 6.1 Hz, 3H). |
| I-140 | ¹H NMR (400 MHz, CDCl₃) δ 8.99 (s, 1H), 8.56 (s, 1H), 8.07 (d, J = 7.8 Hz, |
| | 1H), 7.89 (d, J = 7.6 Hz, 1H), 7.67 (t, J = 7.7 Hz, 1H), 7.23 (t, J = 7.6 Hz, |
| | 1H), 7.08 (d, J = 7.6 Hz, 2H), 6.27 (s, 1H), 5.59-5.52 (m, 1H), 5.43 (d, J = |
| | 15.7 Hz, 1H), 4.44-4.34 (m, 1H), 4.25 (d, J = 16.0 Hz, 1H), 4.07-3.95 (m, |
| | 4H), 2.14-2.08 (m, 1H), 2.05 (s, 6H), 1.85 (d, J = 14.9 Hz, 1H), 1.66-1.50 |
| | (m, 2H), 1.50-1.39 (m, 1H), 1.33-1.28 (m, 2H), 0.54 (s, 3H), 0.42-0.35 (m, |
| | 1H), 0.33-0.26 (m, 1H), 0.21-0.13 (m, 1H), 0.06-0.02 (m, 1H). |
| I-141 | ¹H NMR (400 MHz, CDCl₃) δ 8.96 (t, J = 1.8 Hz, 1H), 8.61 (s, 1H), 8.03- |
| | 7.95 (m, 1H), 7.83 (dt, J = 7.7, 1.4 Hz, 1H), 7.64 (t, J = 7.8 Hz, 1H), 7.24 (t, J = |
| | 7.6 Hz, 1H), 7.08 (d, J = 7.6 Hz, 2H), 6.25 (s, 1H), 5.60 (dd, J = 10.9, 4.0 |
| | Hz, 1H), 5.41 (d, J = 16.0 Hz, 1H), 4.45 (d, J = 16.1 Hz, 1H), 4.20-4.12 (m, |
| | 1H), 4.07 (s, 3H), 4.06-3.98 (m, 1H), 2.21-2.11 (m, 1H), 2.05 (s, 6H), 1.82- |
| | 1.71 (m, 2H), 1.67-1.55 (m, 2H), 1.40-1.33 (m, 2H), 0.68 (s, 9H). |
| I-142 | ¹H NMR (400 MHz, CDCl₃) δ 9.23 (t, J = 1.8 Hz, 1H), 8.04 (d, J = 7.9 Hz, |
| | 1H), 7.84 (dt, J = 7.6, 1.4 Hz, 1H), 7.64 (t, J = 7.8 Hz, 1H), 7.21 (t, J = 7.6 |
| | Hz, 1H), 7.04 (d, J = 7.6 Hz, 2H), 6.87-6.82 (m, 1H), 6.18 (s, 1H), 5.68- |
| | 5.57 (m, 2H), 4.41-4.30 (m, 2H), 3.66 (t, J = 11.4 Hz, 1H), 3.20 (hept, J = |
| | 6.9 Hz, 1H), 1.98 (s, 6H), 1.86-1.79 (m, 1H), 1.46-1.36 (m, 7H), 0.62 (s, |
| | 3H), 0.45-0.36 (m, 1H), 0.35-0.26 (m, 1H), 0.21-0.13 (m, 1H), 0.11-0.07 |
| | (m, 1H). |
| I-144 | ¹H NMR (400 MHz, CDCl₃) δ 9.80 (s, 1H), 8.66 (t, J = 1.8 Hz, 1H), 8.38 (s, |
| | 1H), 7.82 (dt, J = 7.6, 1.4 Hz, 1H), 7.77 (d, J = 7.9 Hz, 1H), 7.58 (t, J = 7.8 |
| | Hz, 1H), 7.23 (t, J = 7.6 Hz, 1H), 7.06 (d, J = 7.6 Hz, 2H), 6.64 (s, 1H), 6.21 |
| | (s, 1H), 5.42-5.31 (m, 2H), 4.57 (d, J = 15.1 Hz, 1H), 4.48 (t, J = 11.6 Hz, |
| | 1H), 4.39-4.28 (m, 1H), 3.66 (dd, J = 10.7, 7.8 Hz, 1H), 3.59 (dd, J = 10.7, |
| | 3.3 Hz, 1H), 3.54-3.40 (m, 1H), 2.67-2.54 (m, 2H), 2.16-2.04 (m, 4H), |
| | 2.04 (s, 6H), 1.62 (s, 3H), 1.12 (d, J = 6.1 Hz, 3H), 1.07 (d, J = 6.0 Hz, 3H). |
| I-145 | ¹H NMR (400 MHz, Chloroform-d) δ 8.64 (s, 1H), 8.35 (s, 1H), 8.06 (d, J = |
| | 7.9 Hz, 1H), 7.80 (d, J = 7.6 Hz, 1H), 7.63 (t, J = 7.7 Hz, 1H), 7.20 (t, J = 7.6 |
| | Hz, 1H), 7.03 (d, J = 7.5 Hz, 2H), 6.61 (s, 1H), 6.19 (s, 1H), 5.39-5.23 (m, |
| | 2H), 4.27 (t, J = 12.3 Hz, 2H), 4.18-4.03 (m, 1H), 2.19-2.07 (m, 1H), 1.99 |
| | (s, 6H), 1.91-1.84 (m, 1H), 1.80-1.73 (m, 1H), 1.20-1.12 (m, 4H), 1.09- |
| | 0.99 (m, 2H), 0.52-0.38 (m, 1H), 0.37-0.25 (m, 2H), −0.05-−0.19 (m, 2H). |
| | (Sulfonamide-NH not visible) |
| I-146 | ¹H NMR (400 MHz, Chloroform-d) δ 8.65 (t, J = 1.9 Hz, 1H), 8.41 (s, 1H), |
| | 8.07 (d, J = 7.9 Hz, 1H), 7.81 (dt, J = 7.7, 1.3 Hz, 1H), 7.63 (t, J = 7.8 Hz, |
| | 1H), 7.20 (t, J = 7.6 Hz, 1H), 7.03 (d, J = 7.6 Hz, 2H), 6.62 (d, J = 1.1 Hz, |
| | 1H), 6.20 (s, 1H), 5.41-5.28 (m, 2H), 4.35-4.22 (m, 2H), 4.19-4.08 (m, |
| | 1H), 3.16 (hept, J = 6.9, 1.1 Hz, 1H), 2.00 (s, 6H), 1.91-1.83 (m, 1H), 1.82- |
| | 1.75 (m, 1H), 1.40 (d, J = 6.9 Hz, 6H), 1.09-1.00 (m, 2H), 0.50-0.38 (m, |
| | 1H), 0.36-0.25 (m, 2H), −0.06-−0.18 (m, 2H). Sulfonamide NH not visible |
| I-150 | ¹H NMR (400 MHz, MeOD) δ 8.95 (t, J = 1.7 Hz, 1H), 8.43 (s, 1H), 8.05 (dt, |
| | J = 7.6, 1.6 Hz, 1H), 7.75 (dt, J = 7.7, 1.5 Hz, 1H), 7.70 (t, J = 7.6 Hz, 1H), |
| | 7.28 (t, J = 7.6 Hz, 1H), 7.15 (d, J = 7.7 Hz, 2H), 6.30 (s, 1H), 6.26 (d, J = 0.8 |
| | Hz, 1H), 5.78 (dd, J = 10.8, 4.0 Hz, 1H), 5.21 (d, J = 16.0 Hz, 1H), 4.72 (d, J = |
| | 16.0 Hz, 1H), 4.31 (t, J = 11.2 Hz, 1H), 4.25-4.15 (m, 1H), 4.05 (s, 3H), |
| | 2.38-2.13 (m, 1H), 2.10 (s, 6H), 1.91 (dd, J = 15.2, 8.8 Hz, 1H), 1.65 (d, J = |
| | 14.4 Hz, 1H), 1.23-1.15 (m, 2H), 0.94-0.86 (m, 2H), 0.71 (s, 9H). |
| I-151 | ¹H NMR (400 MHz, MeOD) δ 8.74 (t, J = 1.8 Hz, 1H), 8.56 (s, 1H), 8.00 (dt, |
| | J = 7.5, 1.7 Hz, 1H), 7.72 (dt, J = 7.6, 1.6 Hz, 1H), 7.68 (t, J = 7.6 Hz, 1H), |
| | 7.28 (t, J = 7.6 Hz, 1H), 7.15 (d, J = 7.6 Hz, 2H), 6.43 (s, 1H), 6.29 (s, 1H), |
| | 5.69 (dd, J = 11.1, 4.3 Hz, 1H), 5.28 (d, J = 15.9 Hz, 1H), 4.94 (d, J = 15.9 |
| | Hz, 1H), 4.61 (t, J = 11.4 Hz, 1H), 4.38-4.27 (m, 1H), 4.14 (s, 3H), 3.82 (dd, |
| | J = 11.0, 8.2 Hz, 1H), 3.66 (dd, J = 11.0, 3.3 Hz, 1H), 3.52 (hept, J = 6.1 Hz, |
| | 1H), 2.33 (tt, J = 8.3, 5.1 Hz, 1H), 2.14 (s, 6H), 1.40-1.31 (m, 2H), 1.12 (d, J = |
| | 6.0 Hz, 3H), 1.09-1.03 (m, 2H), 1.06 (d, J = 6.0 Hz, 3H). |
| I-152 | ¹H NMR (400 MHz, Chloroform-d) δ 8.81-8.76 (m, 1H), 8.36 (s, 1H), 8.10 |
| | (dt, J = 7.9, 1.4 Hz, 1H), 7.84 (dt, J = 7.7, 1.4 Hz, 1H), 7.64 (t, J = 7.8 Hz, |
| | 1H), 7.23 (t, J = 7.8 Hz, 1H), 7.03 (d, J = 7.5 Hz, 1H), 6.77 (d, J = 7.9 Hz, |
| | 1H), 6.62 (s, 1H), 5.45 (dd, J = 11.1, 4.2 Hz, 1H), 5.25 (d, J = 14.9 Hz, 1H), |
| | 4.40-4.30 (m, 2H), 4.28-4.16 (m, 1H), 2.16-2.07 (m, 1H), 1.88-1.80 (m, |
| | 4H), 1.79 (s, 3H), 1.59 (d, J = 15.0 Hz, 1H), 1.47-1.38 (m, 1H), 1.21-1.08 |
| | (m, 4H), 0.88-0.70 (m, 3H), 0.65-0.51 (m, 10H). |
| I-153 | ¹H NMR (400 MHz, CDCl₃) δ 9.33 (s, 1H), 8.66 (t, J = 1.8 Hz, 1H), 8.39 (s, |
| | 1H), 8.07 (d, J = 7.9 Hz, 1H), 7.84 (dt, J = 7.7, 1.3 Hz, 1H), 7.65 (t, J = 7.8 |
| | Hz, 1H), 7.20 (t, J = 7.6 Hz, 1H), 7.04 (d, J = 7.6 Hz, 2H), 6.61 (d, J = 1.1 |
| | Hz, 1H), 6.19 (s, 1H), 5.38-5.29 (m, 2H), 5.26 (d, J = 15.0 Hz, 1H), 4.71- |
| | 4.61 (m, 1H), 4.48-4.35 (m, 2H), 3.23-3.10 (m, 1H), 2.73-2.51 (m, 2H), |
| | 2.36-2.22 (m, 1H), 2.00 (s, 6H), 1.96-1.82 (m, 2H), 1.82-1.70 (m, 1H), |
| | 1.41 (d, J = 6.9 Hz, 6H). |
| I-155 | ¹H NMR (400 MHz, CDCl₃) δ 8.89 (t, J = 1.8 Hz, 1H), 8.53 (s, 1H), 7.93- |
| | 7.87 (m, 1H), 7.81 (dt, J = 7.6, 1.3 Hz, 1H), 7.62 (t, J = 7.8 Hz, 1H), 7.22 (t, J = |
| | 7.6 Hz, 1H), 7.04 (d, J = 7.6 Hz, 2H), 6.19 (s, 1H), 5.52-5.43 (m, 2H), 4.29 |
| | (d, J = 15.8 Hz, 1H), 4.07-3.92 (m, 6H), 2.13-2.05 (m, 1H), 2.03-1.99 (m, |
| | 6H), 1.82-1.74 (m, 2H), 1.48-1.40 (m, 2H), 1.26-1.22 (m, 3H), 0.95 (s, |
| | 3H), 0.24-0.12 (m, 4H). |
| I-156 | ¹H NMR (400 MHz, Chloroform-d) δ 8.70 (t, J = 1.8 Hz, 1H), 8.35 (s, 1H), |
| | 8.11 (d, J = 8.0 Hz, 1H), 7.86 (d, J = 8.0 Hz, 1H), 7.66 (t, J = 7.8 Hz, 1H), |
| | 7.23 (t, J = 7.6 Hz, 1H), 7.08 (d, J = 7.6 Hz, 2H), 6.62 (s, 1H), 6.28 (s, 1H), |
| | 5.42 (dd, J = 11.2, 4.1 Hz, 1H), 5.29 (d, J = 14.9 Hz, 1H), 4.38-4.25 (m, 2H), |
| | 4.14 (t, J = 11.0 Hz, 1H), 2.17-2.10 (m, 1H), 2.06 (s, 6H), 1.86-1.79 (m, |
| | 1H), 1.52-1.44 (m, 1H), 1.37 (ddd, J = 13.7, 10.4, 3.0 Hz, 1H), 1.28-1.24 |
| | (m, 1H), 1.19-1.13 (m, 3H), 0.87-0.79 (m, 3H), 0.40 (d, J = 6.3 Hz, 3H). |
| I-158 | ¹H NMR (400 MHz, CDCl₃) δ 9.08 (t, J = 1.8 Hz, 1H), 8.48 (s, 1H), 8.07 (dt, |
| | J = 8.0, 1.5 Hz, 1H), 7.93-7.82 (m, 2H), 7.61 (t, J = 7.8 Hz, 1H), 7.23 (t, J = |
| | 7.6 Hz, 1H), 7.11 (d, J = 7.6 Hz, 1H), 7.06 (d, J = 7.6 Hz, 1H), 6.48 (s, 1H), |
| | 5.37 (d, J = 15.9 Hz, 1H), 4.43 (d, J = 15.9 Hz, 1H), 4.09-3.98 (m, 1H), 3.89 |
| | (s, 3H), 3.27 (m, 4H), 3.24-3.11 (m, 2H), 2.85-2.76 (m, 1H), 2.09 (s, 3H), |
| | 2.01-1.91 (m, 1H), 1.88 (s, 3H), 1.61 (dd, J = 15.0, 9.4 Hz, 1H), 1.39 (d, J = |
| | 6.8 Hz, 3H), 1.38-1.32 (m, 4H), 0.62 (s, 9H). |
| I-162 | ¹H NMR (400 MHz, Chloroform-d) δ 8.69 (t, J = 1.6 Hz, 1H), 8.48 (s, 1H), |
| | 7.98-7.88 (m, 1H), 7.85 (d, J = 7.6 Hz, 1H), 7.63 (t, J = 7.8 Hz, 1H), 7.22 (t, |
| | J = 7.6 Hz, 1H), 7.08 (d, J = 7.6 Hz, 2H), 6.27 (s, 1H), 5.40 (dd, J = 11.4, 4.5 |
| | Hz, 1H), 5.33 (d, J = 15.2 Hz, 1H), 4.68 (d, J = 15.2 Hz, 1H), 4.60 (t, J = 11.6 |
| | Hz, 1H), 4.37 (ddt, J = 11.9, 7.9, 4.1 Hz, 1H), 3.70-3.58 (m, 2H), 3.49-3.40 |
| | (m, 2H), 2.08 (s, 6H), 1.42 (d, J = 7.0, 1.2 Hz, 6H), 1.10 (d, J = 6.0 Hz, 3H), |
| | 1.04 (d, J = 6.1 Hz, 3H). Sulfonamide-NH not visible |
| I-163 | ¹H NMR (400 MHz, Chloroform-d) δ 8.63 (d, J = 1.8 Hz, 1H), 8.36 (s, 1H), |
| | 8.20 (d, J = 7.9 Hz, 1H), 7.91-7.86 (m, 1H), 7.71 (t, J = 7.7 Hz, 1H), 7.20 (t, |
| | J = 7.6 Hz, 1H), 7.04 (d, J = 7.6 Hz, 2H), 6.60 (d, J = 1.2 Hz, 1H), 6.13 (s, |
| | 1H), 5.39 (dd, J = 11.5, 4.1 Hz, 1H), 5.25 (d, J = 14.8 Hz, 1H), 4.41 (t, J = |
| | 11.5 Hz, 1H), 4.21 (d, J = 14.9 Hz, 1H), 3.95 (td, J = 10.7, 4.0 Hz, 1H), 2.79 |
| | (q, J = 8.8 Hz, 1H), 2.13 (ddd, J = 13.6, 8.1, 5.2 Hz, 1H), 2.00 (s, 6H), 1.83 |
| | (q, J = 6.2, 4.6 Hz, 1H), 1.72 (t, J = 9.2 Hz, 1H), 1.19-1.13 (m, 4H), 1.09 (s, |
| | 3H), 0.89-0.83 (m, 2H), 0.78 (s, 3H). Sulfonamide-NH not visible |
| I-164 | ¹H NMR (400 MHz, Chloroform-d) δ 8.64 (t, J = 1.8 Hz, 1H), 8.36 (s, 1H), |
| | 8.18 (d, J = 7.9 Hz, 1H), 7.90 (dt, J = 7.5, 1.4 Hz, 1H), 7.71 (t, J = 7.8 Hz, |
| | 1H), 7.21 (t, J = 7.6 Hz, 1H), 7.05 (d, J = 7.6 Hz, 2H), 6.61 (s, 1H), 6.15 (s, |
| | 1H), 5.39 (dd, J = 11.5, 4.1 Hz, 1H), 5.25 (d, J = 14.9 Hz, 1H), 4.42 (t, J = |
| | 11.5 Hz, 1H), 4.21 (d, J = 14.8 Hz, 1H), 3.95 (td, J = 10.9, 4.0 Hz, 1H), 2.80 |
| | (h, J = 8.8 Hz, 1H), 2.13 (tt, J = 8.1, 5.3 Hz, 1H), 2.02 (s, 6H), 1.87-1.81 (m, |
| | 1H), 1.74-1.69 (m, 1H), 1.16 (ddd, J = 10.4, 6.5, 2.4 Hz, 4H), 1.09 (s, 3H), |
| | 0.90-0.83 (m, 2H), 0.78 (s, 3H). Sulfonamide-NH not visible |
| I-167 | ¹H NMR (400 MHz, CDCl₃) δ 9.08 (t, J = 1.8 Hz, 1H), 8.59 (s, 1H), 8.10 (dt, |
| | J = 7.9, 1.4 Hz, 1H), 7.87 (dt, J = 7.7, 1.4 Hz, 1H), 7.66 (t, J = 7.8 Hz, 1H), |
| | 7.23 (t, J = 7.6 Hz, 1H), 7.11 (d, J = 7.6 Hz, 1H), 7.06 (d, J = 7.5 Hz, 1H), |
| | 5.65 (dd, J = 11.0, 4.2 Hz, 1H), 5.46 (d, J = 16.0 Hz, 1H), 4.43 (d, J = 16.1 |
| | Hz, 1H), 4.28-4.17 (m, 1H), 4.05 (t, J = 11.3 Hz, 1H), 3.93 (s, 3H), 3.29 |
| | (hept, J = 6.8 Hz, 1H), 2.03 (s, 3H), 1.85 (s, 3H), 1.79 (dd, J = 15.0, 8.5 Hz, |
| | 1H), 1.73 (s, 3H), 1.61 (dd, J = 15.0, 1.7 Hz, 1H), 1.50 (d, J = 6.8 Hz, 3H), |
| | 1.47 (d, J = 6.9 Hz, 3H), 0.66 (s, 9H). |
| I-168 | ¹H NMR (400 MHz, CDCl₃) δ 9.06 (t, J = 1.9 Hz, 1H), 8.48 (s, 1H), 8.08 (dt, |
| | J = 8.2, 1.3 Hz, 1H), 7.86 (dt, J = 7.7, 1.4 Hz, 1H), 7.64 (t, J = 7.8 Hz, 1H), |
| | 7.22 (t, J = 7.6 Hz, 1H), 7.10 (d, J = 7.6 Hz, 1H), 7.06 (d, J = 7.6 Hz, 1H), |
| | 6.47 (s, 1H), 5.64 (dd, J = 11.0, 4.2 Hz, 1H), 5.42 (d, J = 15.9 Hz, 1H), 4.42 |
| | (d, J = 15.8 Hz, 1H), 4.28-4.21 (m, 1H), 4.10 (t, J = 11.3 Hz, 1H), 3.92 (s, |
| | 3H), 3.16 (hept, J = 6.8 Hz, 1H), 2.02 (s, 3H), 1.84 (s, 3H), 1.83-1.76 (m, |
| | 1H), 1.73 (s, 3H), 1.65-1.57 (m, 1H), 1.39 (d, J = 6.8 Hz, 3H), 1.37 (d, J = |
| | 6.8 Hz, 3H), 0.65 (s, 9H). |
| I-170 | ¹H NMR (400 MHz, CDCl₃) δ 9.01 (t, J = 2.9 Hz, 1H), 8.59 (s, 1H), 8.15- |
| | 8.09 (m, 1H), 7.90-7.84 (m, 1H), 7.67 (t, J = 7.8 Hz, 1H), 7.29 (t, J = 7.6 Hz, |
| | 1H), 7.14 (d, J = 7.7 Hz, 1H), 7.08 (d, J = 7.6 Hz, 1H), 5.70-5.60 (m, 1H), |
| | 5.46 (d, J = 14.5 Hz, 1H), 4.53-4.40 (m, 2H), 4.25-3.96 (m, 7H), 2.22 (d, J = |
| | 7.3 Hz, 2H), 2.17-2.07 (m, 1H), 1.84 (s, 3H), 1.74 (s, 3H), 1.68-1.60 (m, |
| | 2H), 1.35-1.30 (m, 2H), 0.83 (d, J = 6.6 Hz, 3H), 0.81 (d, J = 6.5 Hz, 3H), |
| | 0.66 (s, 9H). |
| I-171 | ¹H NMR (400 MHz, CDCl₃) δ 9.10 (t, J = 1.8 Hz, 1H), 8.58 (s, 1H), 8.11 (dt, |
| | J = 8.1, 1.3 Hz, 1H), 7.87 (dt, J = 7.7, 1.4 Hz, 1H), 7.65 (t, J = 7.8 Hz, 1H), |
| | 7.25 (t, J = 7.6 Hz, 1H), 7.12 (d, J = 7.7 Hz, 1H), 7.06 (d, J = 7.5 Hz, 1H), |
| | 5.64 (dd, J = 11.0, 4.2 Hz, 1H), 5.48 (d, J = 16.0 Hz, 1H), 4.44 (d, J = 16.1 |
| | Hz, 1H), 4.27-4.17 (m, 1H), 4.02 (t, J = 11.3 Hz, 1H), 3.93 (s, 3H), 3.27 |
| | (hept, J = 6.8 Hz, 1H), 2.27-2.11 (m, 3H), 1.80 (s, 3H), 1.79-1.75 (m, 1H), |
| | 1.71 (s, 3H), 1.65-1.57 (m, 1H), 1.49 (d, J = 6.8 Hz, 3H), 1.46 (d, J = 6.8 Hz, |
| | 3H), 0.81 (d, J = 4.2 Hz, 3H), 0.79 (d, J = 4.1 Hz, 3H), 0.66 (s, 9H). |
| I-172 | ¹H NMR (400 MHz, CDCl₃) δ 9.04 (t, J = 1.8 Hz, 1H), 8.47 (s, 1H), 8.03 (dt, |
| | J = 7.9, 1.5 Hz, 1H), 7.89 (dt, J = 7.7, 1.5 Hz, 1H), 7.61 (t, J = 7.8 Hz, 1H), |
| | 7.21 (t, J = 7.6 Hz, 1H), 7.07 (d, J = 7.6 Hz, 2H), 6.73 (s, 1H), 6.54 (s, 1H), |
| | 5.36 (d, J = 15.8 Hz, 1H), 4.44 (d, J = 15.9 Hz, 1H), 4.04-3.95 (m, 1H), 3.91 |
| | (s, 3H), 3.29-3.10 (m, 3H), 2.82-2.69 (m, 1H), 2.01 (s, 1H), 1.94 (s, 6H), |
| | 1.62 (dd, J = 15.0, 9.7 Hz, 1H), 1.40 (d, J = 6.8 Hz, 3H), 1.37 (d, J = 6.8 Hz, |
| | 3H), 1.34-1.28 (m, 1H), 0.61 (s, 9H). |
| I-173 | ¹H NMR (400 MHz, CDCl₃) δ 8.81 (t, J = 1.8 Hz, 1H), 8.43 (s, 1H), 8.02 (dt, |
| | J = 8.0, 1.5 Hz, 1H), 7.89-7.85 (m, 1H), 7.60 (t, J = 7.8 Hz, 1H), 7.21 (t, J = |
| | 7.6 Hz, 1H), 7.07 (d, J = 7.6 Hz, 2H), 6.74 (s, 1H), 6.62 (d, J = 1.0 Hz, 1H), |
| | 5.24 (d, J = 15.1 Hz, 1H), 4.40 (d, J = 15.1 Hz, 1H), 4.08-3.96 (m, 1H), 3.23- |
| | 3.11 (m, 2H), 3.06-2.94 (m, 1H), 2.84-2.68 (m, 1H), 2.18-2.05 (m, 1H), |
| | 1.95 (s, 6H), 1.67 (dd, J = 15.1, 9.6 Hz, 1H), 1.40 (d, J = 6.9 Hz, 6H), 1.27 |
| | (dd, J = 15.0, 2.4 Hz, 1H), 0.54 (s, 9H). |
| I-174 | ¹H NMR (400 MHz, CDCl₃) δ 8.67 (t, J = 1.8 Hz, 1H), 8.38 (s, 1H), 7.92- |
| | 7.80 (m, 2H), 7.60 (t, J = 7.8 Hz, 1H), 7.23 (t, J = 7.6 Hz, 1H), 7.07 (d, J = |
| | 7.6 Hz, 2H), 6.61 (s, 1H), 6.23 (s, 1H), 5.42-5.30 (m, 2H), 4.60-4.44 (m, |
| | 2H), 4.40-4.30 (m, 1H), 3.66 (dd, J = 10.7, 7.8 Hz, 1H), 3.59 (dd, J = 10.6, |
| | 3.3 Hz, 1H), 3.54-3.42 (m, 1H), 2.05 (s, 6H), 1.43 (s, 9H), 1.12 (d, J = 6.1 |
| | Hz, 3H), 1.07 (d, J = 6.1 Hz, 3H). |
| I-178 | ¹H NMR (400 MHz, Chloroform-d) δ 9.07 (t, J = 1.8 Hz, 1H), 8.48 (s, 1H), |
| | 8.10 (dt, J = 8.0, 1.4 Hz, 1H), 7.87 (dt, J = 7.6, 1.4 Hz, 1H), 7.65 (t, J = 7.8 |
| | Hz, 1H), 7.25-7.21 (m, 1H), 7.11 (d, J = 7.7 Hz, 1H), 7.05 (d, J = 7.5 Hz, |
| | 1H), 6.45 (s, 1H), 5.61 (dd, J = 11.1, 4.1 Hz, 1H), 5.44 (d, J = 15.8 Hz, 1H), |
| | 4.41 (d, J = 15.8 Hz, 1H), 4.28-4.14 (m, 1H), 4.08 (t, J = 11.3 Hz, 1H), 3.92 |
| | (s, 3H), 3.16 (h, J = 6.8 Hz, 1H), 2.28-2.08 (m, 2H), 1.86-1.77 (m, 4H), |
| | 1.71 (s, 3H), 1.66-1.57 (m, 5H), 1.53-1.47 (m, 2H), 1.41-1.35 (m, 6H), |
| | 1.17-1.08 (m, 3H), 0.83-0.71 (m, 2H), 0.65 (s, 9H). |
| I-179 | ¹H NMR (400 MHz, CDCl₃) δ 9.07 (t, J = 1.8 Hz, 1H), 8.48 (s, 1H), 8.10 (dt, |
| | J = 7.9, 1.5 Hz, 1H), 7.87 (dt, J = 7.6, 1.4 Hz, 1H), 7.64 (t, J = 7.8 Hz, 1H), |
| | 7.31 (t, J = 7.6 Hz, 1H), 7.26-7.20 (m, 1H), 7.06 (d, J = 7.4 Hz, 1H), 6.52 (s, |
| | 1H), 5.64 (dd, J = 10.9, 4.2 Hz, 1H), 5.42 (d, J = 15.6 Hz, 1H), 4.42 (d, J = |
| | 15.7 Hz, 1H), 4.30-4.21 (m, 1H), 4.11 (t, J = 11.3 Hz, 1H), 3.93 (s, 3H), 3.16 |
| | (h, J = 6.8 Hz, 1H), 2.49 (h, J = 6.8 Hz, 1H), 1.82-1.79 (m, 4H), 1.73 (s, 3H), |
| | 1.59 (d, 1H), 1.40 (d, J = 6.8 Hz, 3H), 1.37 (d, J = 6.8 Hz, 3H), 1.14 (d, J = |
| | 6.8 Hz, 3H), 1.09 (d, J = 6.9 Hz, 3H), 0.65 (s, 9H). |
| I-180 | ¹H NMR (400 MHz, CDCl₃) δ 9.06 (t, J = 1.8 Hz, 1H), 8.49 (s, 1H), 7.93 |
| | (d, J = 7.9 Hz, 1H), 7.91-7.84 (m, 1H), 7.63 (t, J = 7.8 Hz, 1H), 7.32 (t, J = 7.7 |
| | Hz, 1H), 7.19 (d, J = 7.8 Hz, 1H), 7.12 (d, J = 7.5 Hz, 1H), 6.42 (s, 1H), 5.61 |
| | (dd, J = 11.1, 4.3 Hz, 1H), 5.42 (d, J = 15.7 Hz, 1H), 4.40 (d, J = 15.7 Hz, |
| | 1H), 4.33-4.23 (m, 1H), 4.12 (t, J = 11.4 Hz, 1H), 3.89 (s, 3H), 3.14 (hept, J = |
| | 6.8 Hz, 1H), 2.19 (hept, J = 6.7 Hz, 1H), 2.03 (s, 3H), 1.79 (dd, J = 15.0, |
| | 8.5 Hz, 1H), 1.72 (s, 3H), 1.61 (s, 1H), 1.39 (d, J = 6.8 Hz, 3H), 1.36 (d, J = |
| | 6.8 Hz, 3H), 0.97 (d, J = 6.8 Hz, 6H), 0.63 (s, 9H). |
| I-181 | ¹H NMR (400 MHz, CDCl₃) δ 8.70 (t, J = 1.8 Hz, 1H), 8.32 (s, 1H), 7.92 (dt, |
| | J = 8.1, 1.4 Hz, 1H), 7.82 (dt, J = 7.7, 1.3 Hz, 1H), 7.60 (t, J = 7.8 Hz, 1H), |
| | 7.22 (t, J = 7.6 Hz, 1H), 7.09 (d, J = 7.6 Hz, 1H), 7.05 (d, J = 7.6 Hz, 1H), |
| | 6.62 (s, 1H), 5.42-5.37 (m, 1H), 5.37-5.31 (m, 1H), 4.61 (d, J = 15.1 Hz, |
| | 1H), 4.51 (t, J = 11.6 Hz, 1H), 4.39-4.28 (m, 1H), 3.72-3.63 (m, 1H), 3.59 |
| | (dd, J = 10.7, 3.2 Hz, 1H), 3.45 (hept, J = 6.1 Hz, 1H), 2.18-2.06 (m, 1H), |
| | 1.97 (s, 3H), 1.94 (s, 3H), 1.69 (s, 3H), 1.20-1.14 (m, 3H), 1.14-1.10 (m, |
| | 4H), 1.06 (d, J = 6.1 Hz, 3H). |
| I-182 | ¹H NMR (400 MHz, CDCl₃) δ 8.95 (t, J = 1.8 Hz, 1H), 8.46 (s, 1H), 7.84 (dt, |
| | J = 7.6, 1.4 Hz, 1H), 7.80 (dt, J = 8.0, 1.4 Hz, 1H), 7.58 (t, J = 7.7 Hz, 1H), |
| | 7.23 (t, J = 7.6 Hz, 1H), 7.10 (d, J = 7.6 Hz, 1H), 7.06 (d, J = 7.5 Hz, 1H), |
| | 6.47 (s, 1H), 5.56-5.47 (m, 2H), 4.66 (d, J = 15.9 Hz, 1H), 4.38-4.33 (m, |
| | 2H), 4.09 (s, 3H), 3.73-3.62 (m, 2H), 3.54 (hept, J = 6.1 Hz, 1H), 1.98 (s, |
| | 3H), 1.97 (s, 3H), 1.68 (s, 3H), 1.50 (s, 9H), 1.16 (d, J = 6.1 Hz, 3H), 1.13 (d, |
| | J = 6.1 Hz, 3H). |
| I-183 | ¹H NMR (400 MHz, MeOD) δ 8.83 (t, J = 1.8 Hz, 1H), 8.47 (s, 1H), 8.00 (dt, |
| | J = 7.0, 1.9 Hz, 1H), 7.73-7.65 (m, 2H), 7.31 (t, J = 7.6 Hz, 1H), 7.20 (d, J = |
| | 7.6 Hz, 1H), 7.17 (d, J = 7.6 Hz, 1H), 6.29 (d, J = 0.8 Hz, 1H), 5.77 (dd, J = |
| | 11.0, 4.1 Hz, 1H), 5.24 (d, J = 15.8 Hz, 1H), 4.70 (d, J = 15.7 Hz, 1H), 4.44 |
| | (t, J = 11.2 Hz, 1H), 4.07 (s, 3H), 4.05-3.97 (m, 1H), 2.24-2.15 (m, 1H), |
| | 2.11 (s, 3H), 1.99 (s, 3H), 1.94-1.81 (m, 1H), 1.79-1.72 (m, 1H), 1.71 (s, |
| | 3H), 1.28-1.18 (m, 3H), 0.95-0.89 (m, 2H), 0.89-0.83 (m, 1H), 0.82 (s, |
| | 9H). |
| I-184 | ¹H NMR (400 MHz, MeOD) δ 8.68 (t, J = 1.8 Hz, 1H), 8.31 (s, 1H), 7.99 (dt, |
| | J = 6.8, 2.0 Hz, 1H), 7.69 (t, J = 1.8 Hz, 1H), 7.66 (t, J = 7.5 Hz, 1H), 7.30 (t, |
| | J = 7.6 Hz, 1H), 7.19 (d, J = 7.6 Hz, 1H), 7.16 (d, J = 7.6 Hz, 1H), 6.77 (s, |
| | 1H), 5.59 (dd, J = 11.0, 4.1 Hz, 1H), 5.14 (d, J = 15.5 Hz, 1H), 4.62 (d, J = |
| | 15.5 Hz, 1H), 4.42 (t, J = 11.3 Hz, 1H), 3.98 (tt, J = 11.2, 3.8 Hz, 1H), 2.28- |
| | 2.18 (m, 1H), 2.11 (s, 3H), 1.97 (s, 3H), 1.92-1.78 (m, 1H), 1.70 (s, 3H), 1.66 |
| | (dt, J = 14.8, 4.1 Hz, 1H), 1.24-1.18 (m, 1H), 1.18-1.14 (m, 2H), 1.14-1.09 |
| | (m, 2H), 0.84-0.79 (m, 1H), 0.78 (s, 9H). |
| I-185 | ¹H NMR (400 MHz, Chloroform-d) δ 8.86 (s, 1H), 8.49 (s, 1H), 7.84 (dd, J = |
| | 14.7, 7.7 Hz, 2H), 7.62 (t, J = 7.8 Hz, 1H), 7.26-7.20 (m, 1H), 7.10 (d, J = |
| | 7.6 Hz, 2H), 6.94 (s, 1H), 6.28 (s, 1H), 5.52 (d, J = 9.4 Hz, 1H), 5.42 (d, J = |
| | 15.8 Hz, 1H), 4.92 (d, J = 16.0 Hz, 1H), 4.50-4.33 (m, 2H), 4.20 (s, 3H), |
| | 3.69-3.61 (m, 2H), 3.54 (hept, J = 6.0 Hz, 1H), 2.09 (s, 6H), 1.54 (s, 9H), |
| | 1.15 (d, J = 5.9 Hz, 3H), 1.12 (d, J = 6.0 Hz, 3H). Sulfonamide NH not visible |
| I-186 | ¹H NMR (400 MHz, Chloroform-d) δ 8.69 (t, J = 1.8 Hz, 1H), 8.31 (s, 1H), |
| | 7.95 (d, J = 7.8 Hz, 1H), 7.85 (dt, J = 7.8, 1.3 Hz, 1H), 7.62 (t, J = 7.8 Hz, |
| | 1H), 7.22 (t, J = 7.6 Hz, 1H), 7.07 (d, J = 7.6 Hz, 2H), 6.64 (s, 1H), 6.25 (s, |
| | 1H), 5.37 (dd, J = 11.4, 4.4 Hz, 1H), 5.33 (d, J = 15.2 Hz, 1H), 4.56 (d, J = |
| | 15.1 Hz, 1H), 4.47 (t, J = 11.6 Hz, 1H), 4.36 (tt, J = 7.8, 3.7 Hz, 1H), 3.66 |
| | (dd, J = 10.7, 7.8 Hz, 1H), 3.58 (dd, J = 10.6, 3.3 Hz, 1H), 3.45 (hept, J = 6.1 |
| | Hz, 1H), 2.06 (s, 6H), 1.54 (s, 3H), 1.40 (q, J = 4.3 Hz, 2H), 1.11 (d, J = 6.1 |
| | Hz, 3H), 1.06 (d, J = 6.1 Hz, 3H), 0.96-0.92 (m, 2H). sulfonamide NH not |
| | visible. |
| I-189 | ¹H NMR (400 MHz, CDCl₃) δ 8.89 (s, 1H), 7.92-7.68 (m, 2H), 7.57 (s, 1H), |
| | 7.27-7.18 (m, 2H), 7.18-6.98 (m, 2H), 6.70 (s, 1H), 5.52 (s, 2H), 4.39 (s, |
| | 2H), 3.98 (s, 3H), 3.86-3.43 (m, 3H), 3.22 (s, 1H), 1.99 (s, 6H), 1.69 (s, 3H), |
| | 1.51-1.28 (m, 6H), 1.15 (d, J = 15.7 Hz, 7H). |
| I-190 | ¹H NMR (400 MHz, Chloroform-d) δ 8.80 (d, J = 1.9 Hz, 1H), 8.50 (s, 1H), |
| | 7.97 (d, J = 7.9 Hz, 1H), 7.50 (t, J = 7.8 Hz, 1H), 7.26-7.16 (m, 5H), 7.09 (d, |
| | J = 7.6 Hz, 2H), 6.87-6.79 (m, 2H), 6.59 (s, 1H), 6.29 (s, 1H), 5.59 (dd, J = |
| | 11.1, 4.0 Hz, 1H), 5.41 (d, J = 15.6 Hz, 1H), 4.45 (dd, J = 22.6, 12.7 Hz, 2H), |
| | 4.28 (t, J = 11.3 Hz, 1H), 3.94 (s, 3H), 3.18 (hept, J = 6.8 Hz, 1H), 3.06 (dd, J = |
| | 14.6, 3.7 Hz, 1H), 2.94 (dd, J = 14.6, 10.7 Hz, 1H), 2.08 (s, 6H), 1.41 (d, J = |
| | 6.7 Hz, 3H), 1.38 (d, J = 6.8 Hz, 3H). Sulfonamide-NH not visible |
| I-192 | ¹H NMR (400 MHz, MeOD) δ 8.67 (t, J = 1.8 Hz, 1H), 8.29 (s, 1H), 7.98 (dt, |
| | J = 7.1, 1.8 Hz, 1H), 7.73-7.62 (m, 2H), 7.30 (t, J = 7.6 Hz, 1H), 7.19 (d, J = |
| | 7.5 Hz, 1H), 7.16 (d, J = 7.7 Hz, 1H), 6.76 (s, 1H), 5.55 (dd, J = 10.9, 4.2 Hz, |
| | 1H), 5.14 (d, J = 15.6 Hz, 1H), 4.58 (d, J = 15.6 Hz, 1H), 4.43 (t, J = 11.3 Hz, |
| | 1H), 4.32-4.21 (m, 1H), 3.83-3.74 (m, 1H), 3.21-3.11 (m, 1H), 3.03-2.93 |
| | (m, 1H), 2.22 (tt, J = 8.3, 5.1 Hz, 1H), 2.11 (s, 3H), 2.00 (s, 3H), 1.99-1.93 |
| | (m, 1H), 1.78 (ddd, J = 14.9, 4.9, 3.4 Hz, 1H), 1.75-1.70 (m, 1H), 1.69 (s, |
| | 3H), 1.47-1.39 (m, 2H), 1.39-1.30 (m, 1H), 1.30-1.22 (m, 1H), 1.20-1.14 |
| | (m, 2H), 1.14-1.10 (m, 2H), 1.10-0.99 (m, 1H). |
| I-193 | ¹H NMR (400 MHz, MeOD) δ 8.73 (t, J = 1.7 Hz, 1H), 8.60 (s, 1H), 8.00 (dt, |
| | J = 6.8, 1.9 Hz, 1H), 7.74-7.64 (m, 2H), 7.32 (t, J = 7.6 Hz, 1H), 7.21 (d, J = |
| | 7.6 Hz, 1H), 7.18 (d, J = 7.6 Hz, 1H), 6.49 (s, 1H), 5.72 (dd, J = 10.9, 4.2 Hz, |
| | 1H), 5.25 (d, J = 15.9 Hz, 1H), 4.74 (d, J = 15.9 Hz, 1H), 4.57 (t, J = 11.2 Hz, |
| | 1H), 4.40-4.27 (m, 1H), 4.18 (s, 3H), 3.89-3.81 (m, 1H), 3.28-3.17 (m, |
| | 1H), 3.10-2.99 (m, 1H), 2.38 (tt, J = 8.3, 5.0 Hz, 1H), 2.12 (s, 3H), 2.08- |
| | 2.03 (m, 1H), 2.03 (s, 3H), 2.03-1.98 (m, 1H), 1.86 (dt, J = 14.9, 3.7 Hz, |
| | 1H), 1.79-1.73 (m, 1H), 1.72 (s, 3H), 1.51-1.44 (m, 2H), 1.43-1.37 (m, |
| | 3H), 1.22-1.14 (m, 1H), 1.14-1.07 (m, 2H). |
| I-194 | ¹H NMR (400 MHz, MeOD) δ 8.76 (t, J = 1.8 Hz, 1H), 8.63 (s, 1H), 7.99 (dt, |
| | J = 7.0, 1.9 Hz, 1H), 7.74-7.64 (m, 2H), 7.32 (t, J = 7.6 Hz, 1H), 7.20 (d, J = |
| | 7.5 Hz, 1H), 7.18 (d, J = 7.8 Hz, 1H), 6.70 (s, 1H), 5.72 (dd, J = 10.8, 4.1 Hz, |
| | 1H), 5.27 (d, J = 16.0 Hz, 1H), 4.74 (d, J = 15.9 Hz, 1H), 4.52 (t, J = 11.2 Hz, |
| | 1H), 4.38-4.27 (m, 1H), 4.08 (s, 3H), 3.89-3.79 (m, 1H), 3.44 (hept, J = 6.7 |
| | Hz, 1H), 3.26-3.17 (m, 1H), 3.10-2.98 (m, 1H), 2.11 (s, 3H), 2.08-2.03 (m, |
| | 1H), 2.02 (s, 3H), 2.01-1.95 (m, 1H), 1.85 (dt, J = 15.0, 3.8 Hz, 1H), 1.79- |
| | 1.72 (m, 1H), 1.71 (s, 3H), 1.51-1.46 (m, 1H), 1.45 (d, J = 6.0 Hz, 3H), 1.44 |
| | (d, J = 6.0 Hz, 3H), 1.42-1.38 (m, 1H), 1.36 (dd, J = 14.1, 3.6 Hz, 1H), 1.21- |
| | 1.09 (m, 1H). |
| I-195 | ¹H NMR (400 MHz, CDCl₃) δ 8.79 (t, J = 1.9 Hz, 1H), 8.35 (s, 1H), 8.08 (dt, |
| | J = 8.1, 1.3 Hz, 1H), 7.84 (dt, J = 7.7, 1.4 Hz, 1H), 7.62 (t, J = 7.8 Hz, 1H), |
| | 7.26-7.21 (m, 1H), 7.16 (d, J = 7.7 Hz, 1H), 7.05 (d, J = 7.4 Hz, 1H), 6.63 (s, |
| | 1H), 5.47 (dd, J = 11.2, 4.3 Hz, 1H), 5.25 (d, J = 14.9 Hz, 1H), 4.39-4.29 (m, |
| | 2H), 4.26-4.16 (m, 1H), 2.32 (dd, J = 7.5, 2.9 Hz, 2H), 2.18-2.08 (m, 1H), |
| | 2.04-1.91 (m, 1H), 1.88-1.82 (m, 1H), 1.80 (s, 3H), 1.74 (s, 3H), 1.71-1.56 |
| | (m, 5H), 1.52-1.45 (m, 2H), 1.18-0.97 (m, 6H), 0.60 (s, 9H). |
| I-198 | ¹H NMR (400 MHz, Chloroform-d) δ 8.98 (t, J = 1.8 Hz, 1H), 7.86 (d, J = 7.6 |
| | Hz, 1H), 7.78 (d, J = 7.9 Hz, 1H), 7.58 (t, J = 7.7 Hz, 1H), 7.39 (d, J = 9.5 |
| | Hz, 1H), 7.23 (t, J = 7.6 Hz, 1H), 7.07 (d, J = 7.6 Hz, 2H), 6.65 (s, 1H), 6.20 |
| | (s, 1H), 5.56 (dd, J = 16.3, 1.8 Hz, 1H), 5.49 (dd, J = 11.2, 4.3 Hz, 1H), 4.59 |
| | (d, J = 16.3 Hz, 1H), 4.27 (dt, J = 9.8, 5.2 Hz, 1H), 4.11 (t, J = 11.5 Hz, 1H), |
| | 3.65 (d, J = 5.1 Hz, 2H), 3.49 (h, J = 6.1 Hz, 1H), 2.09-2.00 (m, 7H), 1.13 |
| | (d, J = 6.1 Hz, 3H), 1.06 (d, J = 6.2 Hz, 3H), 1.05-0.94 (m, 4H). |
| | Sulfonamide NH not visible |
| I-199 | ¹H NMR (400 MHz, Chloroform-d) δ 9.12 (t, J = 1.7 Hz, 1H), 8.67 (d, J = 0.8 |
| | Hz, 1H), 8.10 (d, J = 8.0 Hz, 1H), 7.88 (dt, J = 7.8, 1.3 Hz, 1H), 7.65 (t, J = |
| | 7.8 Hz, 1H), 7.21 (t, J = 7.6 Hz, 1H), 7.07 (d, J = 7.6 Hz, 2H), 6.63 (s, 1H), |
| | 6.23 (s, 1H), 5.58 (dd, J = 11.3, 4.3 Hz, 1H), 5.49 (d, J = 16.7 Hz, 1H), 4.33 |
| | (d, J = 16.7 Hz, 2H), 3.78 (t, J = 11.5 Hz, 1H), 2.19-2.12 (m, 1H), 2.03 (s, |
| | 6H), 1.78 (d, J = 14.8 Hz, 1H), 1.44 (d, J = 4.8 Hz, 1H), 1.21-1.13 (m, 4H), |
| | 0.55 (s, 3H), 0.45-0.39 (m, 1H), 0.29-0.24 (m, 1H), 0.15-0.10 (m, 1H), |
| | 0.06-0.01 (m, 1H). |
| I-200 | ¹H NMR (400 MHz, Chloroform-d) δ 9.03 (s, 1H), 8.67 (s, 1H), 7.85 (d, J = |
| | 7.6 Hz, 2H), 7.59 (t, J = 7.7 Hz, 1H), 7.22 (t, J = 7.7 Hz, 1H), 7.07 (d, J = 7.6 |
| | Hz, 2H), 6.60 (s, 1H), 6.20 (s, 1H), 5.59-5.47 (m, 2H), 4.74 (d, J = 16.8 Hz, |
| | 1H), 4.35-4.25 (m, 1H), 4.11 (t, J = 11.5 Hz, 1H), 3.67-3.58 (m, 2H), 3.49 |
| | (p, J = 5.7 Hz, 1H), 2.20-2.10 (m, 1H), 2.05 (s, 6H), 1.19-1.09 (m, 7H), |
| | 1.05 (d, J = 6.1 Hz, 3H). Sulfonamide NH not visible. |
| I-204 | ¹H NMR (400 MHz, CDCl₃) δ 8.80 (t, J = 1.8 Hz, 1H), 8.36 (s, 1H), 8.09 (dt, |
| | J = 8.0, 1.5 Hz, 1H), 7.85 (dt, J = 7.7, 1.4 Hz, 1H), 7.63 (t, J = 7.8 Hz, 1H), |
| | 7.25-7.20 (m, 1H), 7.13 (d, J = 7.6 Hz, 1H), 7.05 (d, J = 7.5 Hz, 1H), 6.63 (s, |
| | 1H), 5.44 (dd, J = 11.1, 4.2 Hz, 1H), 5.25 (d, J = 14.8 Hz, 1H), 4.42-4.28 (m, |
| | 2H), 4.28-4.18 (m, 1H), 2.39-2.19 (m, 2H), 2.18-2.07 (m, 1H), 1.87-1.81 |
| | (m, 1H), 1.80 (s, 3H), 1.76 (s, 3H), 1.51-1.24 (m, 4H), 1.19-1.12 (m, 4H), |
| | 0.83 (d, J = 6.4 Hz, 6H), 0.60 (s, 9H). |
| I-205 | ¹H NMR (400 MHz, CDCl₃) δ 8.79 (t, J = 1.8 Hz, 1H), 8.35 (s, 1H), 8.05 (dt, |
| | J = 7.9, 1.4 Hz, 1H), 7.82 (dt, J = 7.7, 1.4 Hz, 1H), 7.61 (t, J = 7.8 Hz, 1H), |
| | 7.26-7.20 (m, 1H), 7.09 (t, J = 7.8 Hz, 2H), 6.64 (s, 1H), 5.47 (dd, J = 11.1, |
| | 4.2 Hz, 1H), 5.26 (d, J = 14.9 Hz, 1H), 4.35 (d, J = 14.9 Hz, 1H), 4.28 (t, J = |
| | 11.4 Hz, 1H), 4.20-4.09 (m, 1H), 2.24-2.07 (m, 2H), 2.03-1.91 (m, 4H), |
| | 1.84 (dd, J = 15.2, 9.3 Hz, 1H), 1.74 (s, 3H), 1.63-1.51 (m, 1H), 1.31-1.08 |
| | (m, 7H), 0.68-0.63 (m, 12H), 0.58 (d, J = 6.1 Hz, 3H). |
| I-207 | ¹H NMR (400 MHz, MeOD) δ 8.87 (s, 1H), 8.40 (s, 1H), 8.01 (d, J = 6.7 Hz, |
| | 1H), 7.68-7.63 (m, 1H), 7.43-7.33 (m, 1H), 7.29-7.22 (m, 1H), 7.13 (d, J = |
| | 7.6 Hz, 2H), 6.25 (s, 1H), 6.21 (s, 1H), 5.73 (dd, J = 11.0, 4.0 Hz, 1H), 5.21 |
| | (d, J = 15.9 Hz, 1H), 4.65 (d, J = 15.9 Hz, 1H), 4.29 (t, J = 11.2 Hz, 1H), 4.08 |
| | (t, J = 10.6 Hz, 1H), 4.02 (s, 3H), 2.19-2.15 (m, 1H), 2.14-2.06 (m, 6H), |
| | 2.00-1.78 (m, 3H), 1.28-1.20 (m, 1H), 1.17-1.11 (m, 2H), 0.92 (s, 3H), |
| | 0.87-0.82 (m, 2H), 0.19-0.01 (m, 4H). |
| I-208 | ¹H NMR (400 MHz, Chloroform-d) δ 9.09 (t, J = 1.8 Hz, 1H), 8.11 (d, J = 7.9 |
| | Hz, 1H), 7.89 (dt, J = 7.7, 1.4 Hz, 1H), 7.66 (t, J = 7.8 Hz, 1H), 7.37 (dd, J = |
| | 9.6, 0.9 Hz, 1H), 7.20 (t, J = 7.6 Hz, 1H), 7.05 (d, J = 7.6 Hz, 2H), 6.62 (s, |
| | 1H), 6.17 (s, 1H), 5.61-5.49 (m, 2H), 4.21 (dd, J = 16.2, 1.7 Hz, 1H), 3.98 |
| | (tt, J = 11.1, 3.6 Hz, 1H), 3.71 (t, J = 11.5 Hz, 1H), 2.45 (s, 1H), 2.10-2.04 |
| | (m, 1H), 2.02 (s, 6H), 1.98-1.92 (m, 1H), 1.79 (dd, J = 15.4, 3.3 Hz, 1H), |
| | 1.66-1.59 (m, 6H), 1.08-0.95 (m, 4H). (Sulfonamide NH not visible) |
| I-209 | ¹H NMR (400 MHz, Chloroform-d) δ 9.11 (t, J = 1.8 Hz, 1H), 8.66 (s, 1H), |
| | 8.12 (d, J = 7.9 Hz, 1H), 7.90 (d, J = 7.5 Hz, 1H), 7.67 (t, J = 7.8 Hz, 1H), |
| | 7.20 (t, J = 7.6 Hz, 1H), 7.06 (d, J = 7.7 Hz, 2H), 6.60 (s, 1H), 6.20 (s, 1H), |
| | 5.62 (dd, J = 11.4, 4.0 Hz, 1H), 5.48 (d, J = 16.7 Hz, 1H), 4.35 (d, J = 16.7 |
| | Hz, 1H), 4.02 (ddd, J = 11.1, 7.5, 3.8 Hz, 1H), 3.71 (t, J = 11.5 Hz, 1H), 2.44 |
| | (s, 1H), 2.14 (ddd, J = 8.1, 5.1, 2.8 Hz, 1H), 2.05 (s, 6H), 1.93 (dd, J = 15.5, |
| | 10.9 Hz, 1H), 1.78 (dd, J = 15.5, 3.5 Hz, 1H), 1.59 (s, 6H), 1.21-1.08 (m, |
| | 4H). Sulfonamide NH not visible |
| I-211 | ¹H NMR (400 MHz, CDCl₃) δ 8.95 (t, J = 1.8 Hz, 1H), 8.47 (s, 1H), 8.07 (dt, |
| | J = 8.1, 1.4 Hz, 1H), 7.88 (dt, J = 7.7, 1.4 Hz, 1H), 7.65 (t, J = 7.8 Hz, 1H), |
| | 7.26-7.23 (m, 1H), 7.12 (d, J = 7.7 Hz, 1H), 7.10-7.04 (m, 1H), 6.60 (s, |
| | 1H), 5.55-5.48 (m, 2H), 4.76 (d, J = 15.9 Hz, 1H), 4.43-4.33 (m, 2H), 3.94 |
| | (s, 3H), 3.70-3.60 (m, 2H), 3.52 (hept, J = 6.1 Hz, 1H), 3.19 (hept, J = 6.8 |
| | Hz, 1H), 2.19-2.13 (m, 2H), 1.93 (s, 3H), 1.82-1.74 (m, 1H), 1.69 (s, 3H), |
| | 1.41 (d, J = 6.8 Hz, 3H), 1.39 (d, J = 6.8 Hz, 3H), 1.16 (d, J = 6.0 Hz, 3H), |
| | 1.11 (d, J = 6.1 Hz, 3H), 0.80 (d, J = 2.8 Hz, 3H), 0.79 (d, J = 2.8 Hz, 3H). |
| I-212 | ¹H NMR (400 MHz, CDCl₃) δ 8.94 (t, J = 1.8 Hz, 1H), 8.47 (s, 1H), 8.06 (dt, |
| | J = 7.9, 1.4 Hz, 1H), 7.87 (dt, J = 7.6, 1.4 Hz, 1H), 7.65 (t, J = 7.7 Hz, 1H), |
| | 7.26-7.22 (m, 1H), 7.09 (t, J = 8.0 Hz, 2H), 6.59 (s, 1H), 5.55-5.45 (m, 1H), |
| | 4.73 (d, J = 15.8 Hz, 1H), 4.43-4.32 (m, 2H), 3.94 (s, 3H), 3.72-3.58 (m, |
| | 2H), 3.50 (hept, J = 6.1 Hz, 1H), 3.18 (hept, J = 6.7 Hz, 1H), 2.16-2.01 (m, |
| | 2H), 1.98 (s, 3H), 1.73-1.61 (m, 5H), 1.43-1.37 (m, 6H), 1.15 (d, J = 6.0 |
| | Hz, 3H), 1.10 (d, J = 6.0 Hz, 3H), 0.71 (t, J = 6.5 Hz, 6H). |
| I-216 | ¹H NMR (400 MHz, MeOD) δ 8.69 (t, J = 1.8 Hz, 1H), 8.34 (s, 1H), 7.98 (dt, |
| | J = 7.6, 1.6 Hz, 1H), 7.73-7.62 (m, 2H), 7.30 (t, J = 7.7 Hz, 1H), 7.18 (dd, J = |
| | 12.3, 7.6 Hz, 2H), 6.79 (s, 1H), 5.56 (dd, J = 11.0, 4.5 Hz, 1H), 5.21 (d, J = |
| | 15.7 Hz, 1H), 4.60 (t, J = 11.5 Hz, 1H), 4.30-4.21 (m, 1H), 3.78 (dd, J = |
| | 10.9, 7.8 Hz, 1H), 3.62 (dd, J = 10.8, 3.1 Hz, 1H), 3.43 (hept, J = 6.1 Hz, 1H), |
| | 2.65 (s, 1H), 2.11 (s, 3H), 2.01 (s, 3H), 1.69 (s, 3H), 1.44 (s, 9H), 1.07 (d, J = |
| | 6.1 Hz, 3H), 0.99 (d, J = 6.1 Hz, 3H). |
| I-217 | ¹H NMR (400 MHz, MeOD) δ 8.81 (t, J = 1.8 Hz, 1H), 8.35 (s, 1H), 8.03 (dt, |
| | J = 7.5, 1.6 Hz, 1H), 7.74-7.63 (m, 2H), 7.31 (t, J = 7.6 Hz, 1H), 7.20 (d, J = |
| | 7.6 Hz, 1H), 7.16 (d, J = 7.6 Hz, 1H), 6.80 (s, 1H), 5.65 (dd, J = 10.9, 4.3 Hz, |
| | 1H), 5.15 (d, J = 15.5 Hz, 1H), 4.68 (d, J = 15.6 Hz, 1H), 4.40 (t, J = 11.3 Hz, |
| | 1H), 4.23-4.14 (m, 1H), 2.13 (s, 3H), 2.05-1.93 (m, 1H), 1.91 (s, 3H), 1.72 |
| | (s, 3H), 1.65-1.55 (m, 1H), 1.44 (s, 9H), 0.66 (s, 9H). |
| I-218 | ¹H NMR (400 MHz, Chloroform-d) δ 8.77 (d, J = 1.9 Hz, 1H), 8.40 (s, 1H), |
| | 8.09 (dt, J = 8.0, 1.5 Hz, 1H), 7.86 (dt, J = 7.6, 1.4 Hz, 1H), 7.65 (t, J = 7.8 |
| | Hz, 1H), 7.21 (t, J = 7.6 Hz, 1H), 7.07 (d, J = 7.6 Hz, 2H), 6.62 (s, 1H), 6.28 |
| | (s, 1H), 5.45 (dd, J = 11.2, 4.1 Hz, 1H), 5.25 (d, J = 14.8 Hz, 1H), 4.39-4.27 |
| | (m, 2H), 4.26-4.17 (m, 1H), 2.02 (s, 6H), 1.81 (dd, J = 15.1, 8.7 Hz, 1H), |
| | 1.59 (dd, J = 15.3, 1.6 Hz, 1H), 1.43 (s, 9H), 0.64 (s, 9H). |
| I-219 | ¹H NMR (400 MHz, Chloroform-d) δ 8.66 (t, J = 1.8 Hz, 1H), 8.43 (s, 1H), |
| | 8.09 (d, J = 7.9 Hz, 1H), 7.85 (dt, J = 7.7, 1.4 Hz, 1H), 7.65 (t, J = 7.8 Hz, |
| | 1H), 7.21 (t, J = 7.6 Hz, 1H), 7.05 (d, J = 7.6 Hz, 2H), 6.62 (s, 1H), 6.20 (s, |
| | 1H), 5.45-5.32 (m, 2H), 4.34 (t, J = 11.5 Hz, 1H), 4.28-4.17 (m, 2H), 2.02 |
| | (s, 6H), 1.96-1.88 (m, 1H), 1.43 (s, 9H), 1.33 (dt, J = 11.8, 4.1 Hz, 1H), 0.55- |
| | 0.47 (m, 2H), 0.33-0.24 (m, 1H), 0.14-0.06 (m, 1H), −0.33-−0.44 (m, 1H). |
| I-221 | ¹H NMR (400 MHz, Chloroform-d) δ 8.74 (t, J = 1.8 Hz, 1H), 8.42 (s, 1H), |
| | 8.08 (dt, J = 8.0, 1.5 Hz, 1H), 7.87 (dt, J = 7.7, 1.4 Hz, 1H), 7.63 (t, J = 7.8 |
| | Hz, 1H), 7.22 (t, J = 7.6 Hz, 1H), 7.09 (dd, J = 17.5, 7.6 Hz, 2H), 6.62 (s, 1H), |
| | 5.42 (dd, J = 10.1, 3.3 Hz, 1H), 5.28 (d, J = 14.7 Hz, 1H), 4.51-4.37 (m, 2H), |
| | 4.17 (d, J = 14.7 Hz, 1H), 2.02 (s, 3H), 1.87 (s, 3H), 1.81 (d, J = 15.0 Hz, |
| | 1H), 1.76 (s, 3H), 1.48 (dd, J = 15.0, 9.4 Hz, 1H), 1.43 (s, 9H), 0.44 (s, 3H), |
| | 0.38-0.31 (m, 1H), 0.28-0.21 (m, 1H), 0.16-0.10 (m, 1H), 0.03-0.00 (m, |
| | 1H). |
| I-222 | ¹H NMR (400 MHz, Chloroform-d) δ 9.01 (t, J = 1.9 Hz, 1H), 8.49 (s, 1H), |
| | 8.11 (dt, J = 7.9, 1.5 Hz, 1H), 7.89 (dt, J = 7.9, 1.5 Hz, 1H), 7.66 (t, J = 7.8 |
| | Hz, 1H), 7.22 (t, J = 7.6 Hz, 1H), 7.09 (dd, J = 15.5, 7.5 Hz, 2H), 6.66 (s, 1H), |
| | 5.61 (dd, J = 11.1, 4.4 Hz, 1H), 5.40 (d, J = 15.8 Hz, 1H), 4.48 (t, J = 10.0 |
| | Hz, 1H), 4.28 (d, J = 15.9 Hz, 1H), 4.20-4.08 (m, 4H), 2.03 (s, 3H), 1.87 (s, |
| | 3H), 1.84 (s, 1H), 1.75 (s, 3H), 1.52 (s, 9H), 1.46-1.41 (m, 1H), 0.49 (s, 3H), |
| | 0.42-0.36 (m, 1H), 0.32-0.26 (m, 1H), 0.20-0.13 (m, 1H), 0.07-0.01 (m, |
| | 1H) |
| I-223 | ¹H NMR (400 MHz, Chloroform-d) δ 8.75 (t, J = 1.8 Hz, 1H), 8.42 (s, 1H), |
| | 8.10 (dt, J = 8.2, 1.3 Hz, 1H), 7.90 (dt, J = 7.7, 1.4 Hz, 1H), 7.66 (t, J = 7.8 |
| | Hz, 1H), 7.22 (t, J = 7.6 Hz, 1H), 7.09 (dd, J = 11.6, 7.6 Hz, 2H), 6.62 (s, 1H), |
| | 5.42 (dd, J = 11.4, 4.1 Hz, 1H), 5.32 (d, J = 14.9 Hz, 1H), 4.35 (t, J = 11.5 |
| | Hz, 1H), 4.24 (d, J = 14.9 Hz, 1H), 4.10 (tt, J = 11.2, 3.8 Hz, 1H), 2.44 (s, |
| | 1H), 2.04 (dd, J = 15.4, 10.5 Hz, 1H), 1.99 (s, 3H), 1.95 (s, 3H), 1.77 (dd, J = |
| | 15.5, 3.2 Hz, 1H), 1.74 (s, 3H), 1.57-1.50 (m, 6H), 1.42 (s, 9H). |
| I-224 | ¹H NMR (400 MHz, Chloroform-d) δ 8.79 (t, J = 1.8 Hz, 1H), 8.35 (s, 1H), |
| | 8.08 (dt, J = 8.0, 1.4 Hz, 1H), 7.84 (dt, J = 7.7, 1.4 Hz, 1H), 7.63 (t, J = 7.8 |
| | Hz, 1H), 7.26-7.20 (m, 1H), 7.12 (d, J = 7.7 Hz, 1H), 7.05 (d, J = 7.5 Hz, |
| | 1H), 6.63 (s, 1H), 5.47 (dd, J = 11.2, 4.3 Hz, 1H), 5.25 (d, J = 14.8 Hz, 1H), |
| | 4.39-4.29 (m, 2H), 4.26-4.15 (m, 1H), 2.24 (dd, J = 14.0, 7.2 Hz, 1H), 2.19- |
| | 2.08 (m, 2H), 1.88-1.77 (m, 4H), 1.74 (s, 3H), 1.68-1.61 (m, 2H), 1.61- |
| | 1.55 (m, 3H), 1.54-1.48 (m, 2H), 1.18-1.10 (m, 6H), 0.89-0.75 (m, 3H), |
| | 0.60 (s, 9H). |
| I-226 | ¹H NMR (400 MHz, Chloroform-d) δ 8.72 (s, 1H), 8.64 (s, 1H), 8.10 (dt, J = |
| | 8.0, 1.5 Hz, 1H), 7.89 (dt, J = 7.7, 1.4 Hz, 1H), 7.65 (t, J = 7.8 Hz, 1H), 7.40 |
| | (s, 1H), 7.20 (t, J = 7.6 Hz, 1H), 7.05 (d, J = 7.6 Hz, 2H), 6.66 (s, 1H), 6.20 |
| | (s, 1H), 5.48 (d, J = 15.0 Hz, 1H), 5.13 (s, 1H), 4.38 (t, J = 11.2 Hz, 1H), 4.21 |
| | (s, 2H), 1.99 (s, 6H), 1.78 (d, J = 15.0 Hz, 1H), 1.55-1.50 (m, 4H), 1.31 (s, |
| | 2H), 0.91 (s, 2H), 0.47 (s, 3H), 0.35 (dt, J = 9.7, 5.0 Hz, 1H), 0.22 (dt, J = 9.8, |
| | 5.0 Hz, 1H), 0.11 (dt, J = 9.4, 4.9 Hz, 1H), 0.06-0.00 (m, 1H). |
| I-228 | ¹H NMR (400 MHz, Chloroform-d) δ 9.06 (t, J = 1.9 Hz, 1H), 8.48 (s, 1H), |
| | 8.10 (dt, J = 8.1, 1.4 Hz, 1H), 7.92 (dt, J = 7.8, 1.4 Hz, 1H), 7.68 (t, J = 7.7 |
| | Hz, 1H), 7.22 (t, J = 7.6 Hz, 1H), 7.12-7.05 (m, 2H), 6.46 (s, 1H), 5.62 (dd, J = |
| | 10.5, 3.3 Hz, 1H), 5.44 (d, J = 15.9 Hz, 1H), 4.32 (d, J = 15.9 Hz, 1H), 4.19- |
| | 4.09 (m, 1H), 4.05 (q, J = 11.0 Hz, 1H), 4.01 (s, 3H), 2.45 (s, 1H), 2.01 (s, |
| | 3H), 1.95 (s, 3H), 1.80 (dd, J = 15.5, 3.1 Hz, 1H), 1.72 (s, 3H), 1.61-1.55 (m, |
| | 7H), 1.45 (s, 3H), 1.03-0.92 (m, 2H), 0.89-0.81 (m, 2H). |
| I-229 | ¹H NMR (400 MHz, Chloroform-d) δ 9.07 (t, J = 1.8 Hz, 1H), 8.49 (s, 1H), |
| | 8.16-8.06 (m, 1H), 7.91-7.84 (m, 1H), 7.67 (t, J = 7.8 Hz, 1H), 7.22 (t, J = |
| | 7.6 Hz, 1H), 7.09 (dd, J = 18.4, 7.6 Hz, 2H), 6.57 (s, 1H), 5.67 (dd, J = 11.0, |
| | 4.2 Hz, 1H), 5.41 (d, J = 15.8 Hz, 1H), 4.43 (d, J = 15.8 Hz, 1H), 4.33-4.22 |
| | (m, 1H), 4.12 (t, J = 11.3 Hz, 1H), 4.05 (s, 3H), 2.04 (s, 3H), 1.84 (s, 3H), |
| | 1.82-1.76 (m, 1H), 1.75 (s, 3H), 1.64-1.61 (m, 1H), 1.46 (s, 3H), 1.04-0.96 |
| | (m, 2H), 0.92-0.85 (m, 2H), 0.65 (s, 9H). |
| I-230 | ¹H NMR (400 MHz, Chloroform-d) δ 9.02 (s, 1H), 8.49 (s, 1H), 8.11 (d, J = |
| | 7.9 Hz, 1H), 7.89 (d, J = 7.6 Hz, 1H), 7.67 (t, J = 7.8 Hz, 1H), 7.23 (t, J = 7.6 |
| | Hz, 1H), 7.09 (dd, J = 15.6, 7.5 Hz, 2H), 6.64 (s, 1H), 5.62 (dd, J = 11.1, 4.3 |
| | Hz, 1H), 5.39 (d, J = 15.7 Hz, 1H), 4.48 (t, J = 10.0 Hz, 1H), 4.28 (d, J = 15.8 |
| | Hz, 1H), 4.15 (t, J = 11.5 Hz, 1H), 4.07 (s, 3H), 2.04 (s, 3H), 1.87 (s, 3H), |
| | 1.86-1.82 (m, 1H), 1.75 (s, 3H), 1.47 (s, 3H), 1.44-1.39 (m, 1H), 1.07-0.98 |
| | (m, 2H), 0.95-0.90 (m, 2H), 0.49 (s, 3H), 0.38 (dt, J = 9.8, 5.0 Hz, 1H), 0.28 |
| | (dt, J = 9.7, 5.0 Hz, 1H), 0.20-0.14 (m, 1H), 0.07-0.01 (m, 1H). |
| I-231 | ¹H NMR (400 MHz, Chloroform-d) δ 8.74 (t, J = 1.8 Hz, 1H), 8.35 (s, 1H), |
| | 8.10 (ddd, J = 7.9, 1.8, 1.1 Hz, 1H), 7.89 (dt, J = 7.8, 1.3 Hz, 1H), 7.66 (t, J = |
| | 7.8 Hz, 1H), 7.22 (t, J = 7.6 Hz, 1H), 7.09 (dd, J = 11.3, 7.6 Hz, 2H), 6.64 (s, |
| | 1H), 5.41 (dd, J = 11.4, 4.2 Hz, 1H), 5.31 (d, J = 15.0 Hz, 1H), 4.32 (t, J = |
| | 11.5 Hz, 1H), 4.23 (d, J = 15.0 Hz, 1H), 4.08 (tt, J = 11.1, 3.7 Hz, 1H), 2.43 |
| | (s, 1H), 2.04 (dd, J = 15.4, 10.6 Hz, 1H), 1.99 (s, 3H), 1.96 (s, 3H), 1.82-1.74 |
| | (m, 1H), 1.74 (s, 3H), 1.58-1.49 (m, 9H), 1.43-1.38 (m, 2H), 0.97-0.92 (m, |
| | 2H). |
| I-232 | ¹H NMR (400 MHz, Chloroform-d) δ 8.74 (t, J = 1.8 Hz, 1H), 8.35 (s, 1H), |
| | 8.08 (dt, J = 8.1, 1.3 Hz, 1H), 7.86 (dt, J = 7.8, 1.4 Hz, 1H), 7.63 (t, J = 7.8 |
| | Hz, 1H), 7.21 (t, J = 7.6 Hz, 1H), 7.08 (dd, J = 17.9, 7.6 Hz, 2H), 6.65 (s, 1H), |
| | 5.40 (dd, J = 10.9, 4.0 Hz, 1H), 5.27 (d, J = 14.8 Hz, 1H), 4.51-4.42 (m, 1H), |
| | 4.41-4.34 (m, 1H), 4.16 (d, J = 14.8 Hz, 1H), 2.02 (s, 3H), 1.86 (s, 3H), 1.81 |
| | (d, J = 15.0 Hz, 1H), 1.75 (s, 3H), 1.54 (s, 3H), 1.47 (dd, J = 15.1, 9.8 Hz, |
| | 1H), 1.43-1.38 (m, 2H), 0.97-0.92 (m, 2H), 0.43 (s, 3H), 0.38-0.32 (m, |
| | 1H), 0.26-0.20 (m, 1H), 0.15-0.09 (m, 1H), −0.00-−0.04 (m, 1H). |
| I-233 | ¹H NMR (400 MHz, Chloroform-d) δ 9.07 (t, J = 1.8 Hz, 1H), 8.12 (d, J = 7.9 |
| | Hz, 1H), 7.90 (dt, J = 7.7, 1.4 Hz, 1H), 7.67 (t, J = 7.8 Hz, 1H), 7.37 (d, J = |
| | 9.6, 0.9 Hz, 1H), 7.20 (t, J = 7.6 Hz, 1H), 7.05 (d, J = 7.6 Hz, 2H), 6.65 (d, J = |
| | 1.0 Hz, 1H), 6.19 (s, 1H), 5.61-5.50 (m, 2H), 4.22 (dd, J = 16.2, 1.7 Hz, |
| | 1H), 3.99 (ddt, J = 12.2, 8.2, 4.1 Hz, 1H), 3.75 (t, J = 11.4 Hz, 1H), 2.45 (s, |
| | 1H), 2.03 (s, 6H), 1.98 (dd, J = 15.4, 10.8 Hz, 1H), 1.79 (dd, J = 15.5, 3.3 Hz, |
| | 1H), 1.60 (q, J = 1.4 Hz, 6H), 1.49 (s, 3H), 1.26-1.21 (m, 2H), 0.89-0.82 |
| | (m, 2H). |
| I-235 | ¹H NMR (400 MHz, Chloroform-d) δ 8.81 (s, 1H), 8.10 (dt, J = 8.1, 1.4 Hz, |
| | 1H), 7.87 (dt, J = 7.8, 1.4 Hz, 1H), 7.70-7.54 (m, 2H), 7.44-7.32 (m, 1H), |
| | 7.21 (t, J = 7.6 Hz, 1H), 7.07 (dd, J = 19.2, 7.6 Hz, 2H), 6.63 (s, 1H), 5.42 (d, |
| | J = 15.1 Hz, 1H), 5.25 (s, 1H), 4.40 (s, 1H), 4.29-4.14 (m, 2H), 2.01 (s, 3H), |
| | 1.97-1.87 (m, 1H), 1.82 (s, 3H), 1.72 (s, 3H), 1.57-1.56 (m, 1H), 1.53 (s, |
| | 3H), 1.30 (s, 2H), 0.89 (s, 2H), 0.59 (s, 9H). |
| I-236 | ¹H NMR (400 MHz, Chloroform-d) δ 9.14 (t, J = 1.8 Hz, 1H), 8.11 (dt, J = |
| | 8.0, 1.5 Hz, 1H), 7.85 (dt, J = 7.7, 1.5 Hz, 1H), 7.64 (t, J = 7.8 Hz, 1H), 7.36 |
| | (dd, J = 9.6, 0.9 Hz, 1H), 7.21 (t, J = 7.6 Hz, 1H), 7.08 (dd, J = 17.7, 7.6 Hz, |
| | 2H), 6.65 (d, J = 0.9 Hz, 1H), 5.66 (dd, J = 11.3, 4.3 Hz, 1H), 5.47 (dd, J = |
| | 16.1, 1.7 Hz, 1H), 4.32 (dd, J = 16.2, 1.7 Hz, 1H), 4.22-4.14 (m, 1H), 3.96 (t, |
| | J = 11.4 Hz, 1H), 2.02 (s, 3H), 1.84 (s, 3H), 1.83-1.76 (m, 1H), 1.72 (s, 3H), |
| | 1.57 (d, J = 13.2 Hz, 1H), 1.49 (s, 3H), 1.26-1.20 (m, 2H), 0.87-0.81 (m, |
| | 2H), 0.65 (s, 9H). |
| I-238 | ¹H NMR (400 MHz, MeOD) δ 8.81 (t, J = 2.0 Hz, 1H), 8.35 (s, 1H), 8.02 (dt, |
| | J = 7.5, 1.7 Hz, 1H), 7.74-7.64 (m, 2H), 7.35 (t, J = 7.6 Hz, 1H), 7.21 (d, J = |
| | 7.7 Hz, 1H), 7.17 (d, J = 7.6 Hz, 1H), 6.81 (s, 1H), 5.67 (dd, J = 11.0, 4.3 Hz, |
| | 1H), 5.15 (d, J = 15.5 Hz, 1H), 4.68 (d, J = 15.5 Hz, 1H), 4.41 (t, J = 11.3 Hz, |
| | 1H), 4.22-4.13 (m, 1H), 2.32-2.26 (m, 2H), 2.05-1.92 (m, 1H), 1.90 (s, |
| | 3H), 1.80 (hept, J = 6.8 Hz, 1H), 1.71 (s, 3H), 1.60 (d, J = 15.1 Hz, 1H), 1.44 |
| | (s, 9H), 0.86 (d, J = 2.6 Hz, 3H), 0.85 (d, J = 2.5 Hz, 3H), 0.66 (s, 9H). |
| I-239 | ¹H NMR (400 MHz, Chloroform-d) δ 9.06 (t, J = 1.9 Hz, 1H), 8.49 (s, 1H), |
| | 8.09 (dt, J = 8.1, 1.2 Hz, 1H), 7.89 (dt, J = 7.8, 1.3 Hz, 1H), 7.67 (t, J = 7.8 |
| | Hz, 1H), 7.21 (t, J = 7.6 Hz, 1H), 7.07 (d, J = 7.6 Hz, 2H), 6.52 (s, 1H), 6.27 |
| | (s, 1H), 5.63 (dd, J = 11.2, 4.1 Hz, 1H), 5.40 (d, J = 15.6 Hz, 1H), 4.40 (d, J = |
| | 15.7 Hz, 1H), 4.25 (t, J = 8.9 Hz, 1H), 4.13-3.99 (m, 4H), 2.02 (s, 6H), 1.77 |
| | (dd, J = 15.0, 8.7 Hz, 1H), 1.67-1.61 (m, 1H), 1.50 (s, 9H), 0.67 (s, 9H). |
| I-240 | ¹H NMR (400 MHz, Chloroform-d) δ 8.93 (t, J = 1.8 Hz, 1H), 8.47 (s, 1H), |
| | 7.93-7.77 (m, 2H), 7.61 (t, J = 7.8 Hz, 1H), 7.23 (d, J = 7.6 Hz, 1H), 7.09 (d, |
| | J = 7.6 Hz, 2H), 6.50 (s, 1H), 6.25 (s, 1H), 5.55-5.50 (m, 1H), 5.47 (d, J = |
| | 15.9 Hz, 1H), 4.67 (d, J = 15.9 Hz, 1H), 4.41-4.29 (m, 2H), 4.02 (s, 3H), |
| | 3.69-3.59 (m, 2H), 3.54 (h, J = 6.1 Hz, 1H), 2.08 (s, 6H), 1.46 (s, 3H), 1.15 |
| | (d, J = 6.0 Hz, 3H), 1.11 (d, J = 6.1 Hz, 3H), 1.03-0.95 (m, 2H), 0.91-0.81 |
| | (m, 2H). |
| I-241 | ¹H NMR (400 MHz, Chloroform-d) δ 9.27-8.99 (m, 1H), 8.63 (s, 1H), 7.90 |
| | (s, 1H), 7.84 (d, J = 7.9 Hz, 1H), 7.82-7.65 (m, 1H), 7.62 (t, J = 7.8 Hz, 1H), |
| | 7.57-7.39 (m, 1H), 7.22 (t, J = 7.7 Hz, 1H), 7.06 (d, J = 7.6 Hz, 2H), 6.20 (s, |
| | 1H), 5.53 (d, J = 15.6 Hz, 1H), 5.15 (d, J = 10.0 Hz, 1H), 4.87-4.45 (m, 2H), |
| | 4.37 (s, 1H), 3.73 (d, J = 69.5 Hz, 2H), 3.44 (p, J = 6.1 Hz, 1H), 2.05 (s, 6H), |
| | 1.56-1.50 (m, 5H), 1.43-1.28 (m, 2H), 1.08 (d, J = 6.1 Hz, 3H), 0.99 (s, |
| | 3H). |
| I-242 | ¹H NMR (400 MHz, MeOD) δ 8.95 (t, J = 1.8 Hz, 1H), 8.00 (dt, J = 7.5, 1.6 |
| | Hz, 1H), 7.76-7.71 (m, 1H), 7.71-7.69 (m, 1H), 7.66 (t, J = 7.6 Hz, 1H), |
| | 7.27 (t, J = 7.6 Hz, 1H), 7.14 (d, J = 7.6 Hz, 2H), 6.73 (d, J = 0.9 Hz, 1H), |
| | 6.21 (s, 1H), 5.63 (d, J = 7.0 Hz, 1H), 5.38 (dd, J = 16.4, 1.8 Hz, 1H), 4.76 |
| | (dd, J = 16.4, 1.6 Hz, 1H), 4.30-4.16 (m, 2H), 3.82-3.73 (m, 1H), 3.63 (dd, |
| | J = 10.8, 2.4 Hz, 1H), 3.50 (hept, J = 6.1 Hz, 1H), 2.12 (s, 6H), 1.40 (s, 9H), |
| | 1.12 (d, J = 6.1 Hz, 3H), 1.03 (d, J = 6.1 Hz, 3H). |
| I-243 | ¹H NMR (400 MHz, MeOD) δ 9.08 (t, J = 1.8 Hz, 1H), 8.03 (dt, J = 7.8, 1.5 |
| | Hz, 1H), 7.74 (dt, J = 7.6, 1.5 Hz, 1H), 7.72-7.63 (m, 2H), 7.27 (t, J = 7.6 |
| | Hz, 1H), 7.14 (d, J = 7.7 Hz, 2H), 6.74 (d, J = 0.9 Hz, 1H), 6.24 (s, 1H), 5.71 |
| | (dd, J = 11.0, 4.2 Hz, 1H), 5.28 (dd, J = 16.4, 1.9 Hz, 1H), 4.46 (dd, J = 16.5, |
| | 1.5 Hz, 1H), 4.40-4.27 (m, 1H), 4.04 (t, J = 11.4 Hz, 1H), 2.10 (s, 6H), 1.81 |
| | (d, J = 15.0 Hz, 1H), 1.57 (dd, J = 15.3, 10.0 Hz, 1H), 1.40 (s, 9H), 0.57 (s, |
| | 3H), 0.42 (dt, J = 9.5, 4.9 Hz, 1H), 0.28 (dt, J = 9.5, 4.9 Hz, 1H), 0.16 (dt, J = |
| | 9.3, 4.8 Hz, 1H), 0.08 (dt, J = 9.7, 4.9 Hz, 1H). |
| I-244 | ¹H NMR (400 MHz, MeOD) δ 9.08 (t, J = 1.9 Hz, 1H), 8.04 (d, J = 7.8 Hz, |
| | 1H), 7.80-7.74 (m, 1H), 7.73-7.65 (m, 2H), 7.27 (t, J = 7.7 Hz, 1H), 7.14 (d, |
| | J = 7.7 Hz, 2H), 6.74 (d, J = 0.9 Hz, 1H), 6.22 (s, 1H), 5.76 (d, J = 7.6 Hz, |
| | 1H), 5.33 (dd, J = 16.5, 1.9 Hz, 1H), 4.50 (d, J = 16.7 Hz, 1H), 4.05-3.90 (m, |
| | 2H), 2.44 (s, 1H), 2.12 (s, 6H), 2.09-2.02 (m, 1H), 1.83 (dd, J = 15.0, 2.5 Hz, |
| | 1H), 1.63 (s, 6H), 1.40 (s, 9H). |
| I-245 | ¹H NMR (400 MHz, CDCl₃) δ 8.78 (t, J = 1.8 Hz, 1H), 8.42 (s, 1H), 8.08 (dt, |
| | J = 8.1, 1.3 Hz, 1H), 7.84 (dt, J = 7.7, 1.4 Hz, 1H), 7.63 (t, J = 7.8 Hz, 1H), |
| | 7.23 (d, J = 7.6 Hz, 1H), 7.17 (d, J = 7.5 Hz, 1H), 7.08 (d, J = 7.4 Hz, 1H), |
| | 6.63 (s, 1H), 5.48 (dd, J = 11.3, 4.4 Hz, 1H), 5.26 (d, J = 14.8 Hz, 1H), 4.44- |
| | 4.31 (m, 2H), 4.29-4.17 (m, 1H), 2.43 (d, J = 13.5 Hz, 1H), 2.22 (d, J = 13.4 |
| | Hz, 1H), 1.89-1.77 (m, 4H), 1.73 (s, 3H), 1.62-1.58 (m, 1H), 1.42 (s, 9H), |
| | 0.83 (s, 9H), 0.60 (s, 9H). |
| I-246 | ¹H NMR (400 MHz, CDCl₃) δ 8.78 (t, J = 1.8 Hz, 1H), 8.42 (s, 1H), 8.09 (dt, |
| | J = 8.0, 1.5 Hz, 1H), 7.84 (dt, J = 7.8, 1.4 Hz, 1H), 7.63 (t, J = 7.8 Hz, 1H), |
| | 7.23 (d, J = 7.6 Hz, 1H), 7.12 (t, J = 7.6 Hz, 2H), 6.63 (s, 1H), 5.44 (dd, J = |
| | 11.0, 4.1 Hz, 1H), 5.27 (d, J = 14.8 Hz, 1H), 4.42-4.32 (m, 2H), 4.32-4.23 |
| | (m, 1H), 2.28 (d, J = 13.4 Hz, 1H), 2.06-1.97 (m, 4H), 1.90-1.79 (m, 1H), |
| | 1.73 (s, 3H), 1.42 (s, 10H), 0.66 (s, 9H), 0.65 (s, 9H). |
| I-247 | ¹H NMR (400 MHz, CDCl₃) δ 8.75 (t, J = 1.8 Hz, 1H), 8.42 (s, 1H), 8.09 (dt, |
| | J = 8.1, 1.3 Hz, 1H), 7.90 (dt, J = 7.8, 1.4 Hz, 1H), 7.65 (t, J = 7.8 Hz, 1H), |
| | 7.26-7.23 (m, 1H), 7.12 (d, J = 7.7 Hz, 1H), 7.08 (d, J = 7.5 Hz, 1H), 6.63 (s, |
| | 1H), 5.42 (dd, J = 11.4, 4.2 Hz, 1H), 5.33 (d, J = 14.9 Hz, 1H), 4.36 (t, J = |
| | 11.5 Hz, 1H), 4.25 (d, J = 14.9 Hz, 1H), 4.16-4.03 (m, 1H), 2.43 (s, 1H), |
| | 2.25-2.10 (m, 2H), 2.08-2.00 (m, 1H), 1.90 (s, 3H), 1.81-1.76 (m, 1H), |
| | 1.73 (s, 3H), 1.55-1.48 (m, 7H), 1.43 (s, 9H), 0.81 (d, J = 2.4 Hz, 3H), 0.80 |
| | (d, J = 2.4 Hz, 3H). |
| I-248 | ¹H NMR (400 MHz, CDCl₃) δ 8.75 (t, J = 1.8 Hz, 1H), 8.42 (s, 1H), 8.09 (dt, |
| | J = 8.0, 1.4 Hz, 1H), 7.89 (dt, J = 7.8, 1.4 Hz, 1H), 7.65 (t, J = 7.8 Hz, 1H), |
| | 7.26-7.21 (m, 1H), 7.12 (d, J = 7.6 Hz, 1H), 7.06 (d, J = 7.6 Hz, 1H), 6.62 (s, |
| | 1H), 5.42 (dd, J = 11.4, 4.3 Hz, 1H), 5.31 (d, J = 14.9 Hz, 1H), 4.38 (t, J = |
| | 11.6 Hz, 1H), 4.25 (d, J = 14.9 Hz, 1H), 4.20-4.11 (m, 1H), 2.41 (s, 1H), |
| | 2.10-1.98 (m, 6H), 1.77-1.71 (m, 4H), 1.57-1.51 (m, 1H), 1.51-1.44 (m, |
| | 6H), 1.43 (s, 9H), 0.66 (t, J = 6.9 Hz, 6H). |
| I-249 | ¹H NMR (400 MHz, Chloroform-d) δ 8.77 (d, J = 1.8 Hz, 1H), 8.41 (s, 1H), |
| | 8.09 (d, J = 8.2 Hz, 1H), 7.81 (d, J = 7.2 Hz, 1H), 7.62 (t, J = 7.8 Hz, 1H), |
| | 7.24 (s, 1H), 6.80 (d, J = 8.0 Hz, 2H), 6.62 (s, 1H), 5.49 (dd, J = 11.3, 4.3 Hz, |
| | 1H), 5.26 (d, J = 14.8 Hz, 1H), 4.52 (hept, J = 6.1 Hz, 1H), 4.44-4.30 (m, |
| | 2H), 4.28-4.19 (m, 1H), 1.86 (s, 3H), 1.85-1.78 (m, 1H), 1.77 (s, 3H), 1.57 |
| | (d, J = 15.0 Hz, 1H), 1.42 (s, 9H), 1.23 (t, J = 5.6 Hz, 6H), 0.61 (s, 9H). |
| I-250 | ¹H NMR (400 MHz, MeOD) δ 9.04 (t, J = 1.8 Hz, 1H), 8.87 (d, J = 0.8 Hz, |
| | 1H), 8.05 (dt, J = 7.3, 1.7 Hz, 1H), 7.73 (dt, J = 7.6, 1.6 Hz, 1H), 7.69 (t, J = |
| | 7.5 Hz, 1H), 7.27 (t, J = 7.6 Hz, 1H), 7.14 (d, J = 7.7 Hz, 2H), 6.77 (d, J = 0.8 |
| | Hz, 1H), 6.27 (s, 1H), 5.77 (dd, J = 10.5, 3.7 Hz, 1H), 5.19 (d, J = 16.6 Hz, |
| | 1H), 4.72 (d, J = 16.7 Hz, 1H), 4.18 (td, J = 9.9, 3.6 Hz, 1H), 4.14-4.05 (m, |
| | 1H), 2.09 (s, 6H), 1.86 (dd, J = 15.2, 8.7 Hz, 1H), 1.60 (d, J = 15.1 Hz, 1H), |
| | 1.44 (s, 9H), 0.70 (s, 9H). |
| I-251 | ¹H NMR (400 MHz, MeOD) δ 9.03 (t, J = 1.8 Hz, 1H), 8.87 (d, J = 0.8 Hz, |
| | 1H), 8.05 (dt, J = 7.8, 1.5 Hz, 1H), 7.77 (dt, J = 7.7, 1.5 Hz, 1H), 7.70 (t, J = |
| | 7.7 Hz, 1H), 7.27 (t, J = 7.6 Hz, 1H), 7.14 (d, J = 7.6 Hz, 2H), 6.77 (d, J = 0.8 |
| | Hz, 1H), 6.24 (s, 1H), 5.84-5.73 (m, 1H), 5.21 (d, J = 16.7 Hz, 1H), 4.63 (d, |
| | J = 16.7 Hz, 1H), 4.09-3.98 (m, 2H), 2.43 (s, 1H), 2.13 (s, 6H), 2.08-2.00 |
| | (m, 1H), 1.82 (dd, J = 15.7, 2.3 Hz, 1H), 1.61 (s, 6H), 1.44 (s, 9H). |
| I-252 | ¹H NMR (400 MHz, MeOD) δ 9.01 (t, J = 1.8 Hz, 1H), 8.88 (d, J = 0.8 Hz, |
| | 1H), 8.04 (dt, J = 7.7, 1.6 Hz, 1H), 7.74 (dt, J = 7.6, 1.5 Hz, 1H), 7.68 (t, J = |
| | 7.7 Hz, 1H), 7.27 (t, J = 7.6 Hz, 1H), 7.14 (d, J = 7.7 Hz, 2H), 6.78 (d, J = 0.8 |
| | Hz, 1H), 6.26 (s, 1H), 5.73 (dd, J = 11.0, 4.3 Hz, 1H), 5.18 (d, J = 16.6 Hz, |
| | 1H), 4.59 (d, J = 16.7 Hz, 1H), 4.43-4.30 (m, 1H), 4.10 (t, J = 11.4 Hz, 1H), |
| | 2.10 (s, 6H), 1.81 (d, J = 15.0 Hz, 1H), 1.55 (dd, J = 15.2, 10.2 Hz, 1H), 1.44 |
| | (s, 9H), 0.56 (s, 3H), 0.42 (dt, J = 9.6, 4.9 Hz, 1H), 0.28 (dt, J = 9.6, 4.9 Hz, |
| | 1H), 0.16 (dt, J = 9.3, 4.8 Hz, 1H), 0.06 (dt, J = 9.7, 4.9 Hz, 1H). |
| I-253 | ¹H NMR (400 MHz, MeOD) δ 8.94 (t, J = 1.8 Hz, 1H), 8.00 (dt, J = 7.5, 1.7 |
| | Hz, 1H), 7.71 (dt, J = 7.6, 1.6 Hz, 1H), 7.68 (d, J = 7.6 Hz, 1H), 7.65 (d, J = |
| | 7.8 Hz, 1H), 7.27 (t, J = 7.6 Hz, 1H), 7.14 (d, J = 7.7 Hz, 2H), 6.74 (d, J = 0.9 |
| | Hz, 1H), 6.21 (s, 1H), 5.66-5.59 (m, 1H), 5.36 (dd, J = 16.5, 1.8 Hz, 1H), |
| | 4.75 (dd, J = 16.5, 1.6 Hz, 1H), 4.29-4.18 (m, 2H), 3.82-3.73 (m, 1H), 3.62 |
| | (dd, J = 10.8, 2.4 Hz, 1H), 3.49 (hept, J = 6.1 Hz, 1H), 2.12 (s, 6H), 1.52 (s, |
| | 3H), 1.29-1.22 (m, 2H), 1.12 (d, J = 6.0 Hz, 3H), 1.02 (d, J = 6.0 Hz, 3H), |
| | 0.94-0.87 (m, 2H). |
| I-254 | ¹H NMR (400 MHz, MeOD) δ 8.74 (t, J = 1.7 Hz, 1H), 8.42 (s, 1H), 8.02 (dt, |
| | J = 7.8, 1.5 Hz, 1H), 7.75 (dt, J = 7.7, 1.5 Hz, 1H), 7.67 (t, J = 7.7 Hz, 1H), |
| | 7.31-7.23 (m, 2H), 7.14 (d, J = 7.7 Hz, 2H), 6.29 (s, 1H), 5.57 (dd, J = 10.6, |
| | 3.9 Hz, 1H), 5.13 (d, J = 15.7 Hz, 1H), 4.53 (d, J = 15.6 Hz, 1H), 4.45-4.35 |
| | (m, 1H), 4.30 (t, J = 11.2 Hz, 1H), 2.10 (s, 6H), 1.82 (d, J = 15.1 Hz, 1H), |
| | 1.56 (dd, J = 15.3, 9.8 Hz, 1H), 0.50 (s, 3H), 0.39 (dt, J = 9.5, 5.1 Hz, 1H), |
| | 0.25 (dt, J = 9.7, 4.9 Hz, 1H), 0.15 (dt, J = 9.3, 4.8 Hz, 1H), 0.06 (dt, J = 9.5, |
| | 4.9 Hz, 1H). |
| I-255 | ¹H NMR (400 MHz, MeOD) δ 8.78 (t, J = 1.8 Hz, 1H), 8.39 (s, 1H), 8.06- |
| | 7.98 (m, 1H), 7.79-7.72 (m, 1H), 7.68 (t, J = 7.7 Hz, 1H), 7.27 (t, J = 7.7 Hz, |
| | 1H), 7.14 (d, J = 7.7 Hz, 2H), 7.09 (s, 1H), 6.29 (s, 1H), 5.99 (s, 1H), 5.60 |
| | (dd, J = 10.6, 3.8 Hz, 1H), 5.44 (t, J = 1.6 Hz, 1H), 5.14 (d, J = 15.6 Hz, 1H), |
| | 4.52 (d, J = 15.6 Hz, 1H), 4.47-4.36 (m, 1H), 4.32 (t, J = 11.1 Hz, 1H), 2.20 |
| | (s, 3H), 2.10 (s, 6H), 1.83 (d, J = 15.1 Hz, 1H), 1.59 (dd, J = 15.3, 9.8 Hz, |
| | 1H), 0.52 (s, 3H), 0.40 (dt, J = 9.7, 4.4 Hz, 1H), 0.26 (dt, J = 9.5, 4.9 Hz, 1H), |
| | 0.16 (dt, J = 9.3, 4.9 Hz, 1H), 0.07 (dt, J = 9.6, 4.8 Hz, 1H). |
| I-257 | ¹H NMR (400 MHz, CDCl₃) δ 8.79 (t, J = 1.9 Hz, 1H), 8.35 (s, 1H), 8.08 (dt, |
| | J = 7.9, 1.5 Hz, 1H), 7.84 (dt, J = 7.6, 1.4 Hz, 1H), 7.63 (t, J = 7.8 Hz, 1H), |
| | 7.26-7.21 (m, 1H), 7.12 (d, J = 7.7 Hz, 1H), 7.06 (d, J = 7.5 Hz, 1H), 6.65 (s, |
| | 1H), 5.47 (dd, J = 11.2, 4.4 Hz, 1H), 5.25 (d, J = 14.8 Hz, 1H), 4.41-4.32 (m, |
| | 2H), 4.27-4.18 (m, 1H), 2.27-2.12 (m, 2H), 1.87-1.76 (m, 5H), 1.74 (s, |
| | 3H), 1.62-1.59 (m, 1H), 1.54 (s, 3H), 1.43-1.37 (m, 2H), 0.98-0.91 (m, |
| | 2H), 0.82 (d, J = 2.6 Hz, 3H), 0.80 (d, J = 2.6 Hz, 3H), 0.60 (s, 9H). |
| I-263 | ¹H NMR (400 MHz, CDCl₃) δ 9.17 (s, 1H), 8.84 (s, 1H), 8.03 (d, J = 1.4 Hz, |
| | 1H), 7.89 (d, J = 1.4 Hz, 1H), 7.66 (t, J = 7.8 Hz, 1H), 7.22 (t, J = 7.6 Hz, |
| | 1H), 7.06 (d, J = 7.6 Hz, 2H), 6.36 (s, 1H), 6.19 (s, 1H), 5.75 (d, J = 17.6 Hz, |
| | 1H), 5.55 (dd, J = 10.9, 4.2 Hz, 1H), 4.72 (d, J = 17.7 Hz, 1H), 4.44-4.34 (m, |
| | 1H), 4.05 (s, 3H), 3.82 (t, J = 11.3 Hz, 1H), 2.02 (s, 6H), 1.98-1.90 (m, 1H), |
| | 1.83 (d, J = 1.9 Hz, 1H), 1.50 (dd, J = 15.1, 10.1 Hz, 1H), 1.19-1.13 (m, 2H), |
| | 0.92-0.79 (m, 2H), 0.56 (s, 3H), 0.48-0.40 (m, 1H), 0.31-0.24 (m, 1H), |
| | 0.18-0.13 (m, 1H), 0.13-0.06 (m, 1H). |
| II-4 | ¹H NMR (400 MHz, CDCl₃) δ 8.80-8.77 (m, 1H), 8.06 (d, J = 7.9 Hz, 1H), |
| | 7.89-7.84 (m, 3H), 7.66 (t, J = 7.8 Hz, 1H), 7.20 (t, J = 7.6 Hz, 1H), 7.06 (d, |
| | J = 7.6 Hz, 2H), 6.23 (s, 1H), 5.39-5.34 (m, 1H), 5.16 (d, J = 15.3 Hz, 1H), |
| | 4.11-3.96 (m, 3H), 3.62-3.56 (m, 2H), 2.05 (s, 6H), 1.99-1.92 (m, 4H), |
| | 1.60 (s, 6H), 1.20-1.12 (m, 3H), 0.80 (s, 10H). |
| II-5 | ¹H NMR (400 MHz, CDCl₃) δ 8.75 (t, J = 1.8 Hz, 1H), 8.05 (d, J = 7.9 Hz, |
| | 1H), 7.89 (d, J = 8.1 Hz, 2H), 7.81 (dt, J = 7.7, 1.4 Hz, 1H), 7.64 (t, J = 7.8 |
| | Hz, 1H), 7.21 (t, J = 7.6 Hz, 1H), 7.03 (d, J = 7.6 Hz, 2H), 6.15 (s, 1H), 5.35 |
| | (dd, J = 11.2, 4.2 Hz, 1H), 5.19 (d, J = 15.8 Hz, 1H), 4.60-4.47 (m, 1H), 4.34- |
| | 4.21 (m, 1H), 4.10-3.98 (m, 2H), 3.92-3.81 (m, 1H), 3.20 (td, J = 11.2, 3.6 |
| | Hz, 1H), 3.14 (s, 3H), 3.05-2.93 (m, 1H), 2.35-2.26 (m, 2H), 2.25-2.14 (m, |
| | 2H), 2.00 (s, 6H), 1.96-1.86 (m, 1H), 1.79-1.63 (m, 4H), 1.54-1.40 (m, |
| | 2H), 1.39-1.25 (m, 1H), 1.25-1.16 (m, 1H), 1.12-0.99 (m, 1H). |
| II-6 | ¹H NMR (400 MHz, Chloroform-d) δ 14.32 (s, 1H), 8.61 (s, 1H), 7.84 (d, J = |
| | 7.5 Hz, 1H), 7.82-7.73 (m, 2H), 7.61 (t, J = 7.7 Hz, 1H), 7.28-7.23 (m, 1H), |
| | 7.07 (d, J = 7.7 Hz, 2H), 7.03 (d, J = 7.3 Hz, 1H), 6.82 (s, 1H), 6.20 (s, 1H), |
| | 5.65 (d, J = 16.8 Hz, 1H), 5.42 (d, J = 17.0 Hz, 1H), 5.14 (dd, J = 10.7, 4.2 |
| | Hz, 1H), 4.77-4.63 (m, 2H), 4.48-4.38 (m, 1H), 4.03 (t, J = 10.5 Hz, 1H), |
| | 3.61-3.49 (m, 2H), 3.43 (s, 3H), 2.53-2.43 (m, 2H), 2.31-2.22 (m, 2H), |
| | 2.07 (s, 6H), 1.90-1.82 (m, 2H), 1.10 (d, J = 6.0 Hz, 3H), 1.02 (d, J = 6.0 Hz, |
| | 3H). |
| II-9 | ¹H NMR (400 MHz, CDCl₃) δ 8.79 (s, 1H), 7.86-7.80 (m, 2H), 7.64-7.54 |
| | (m, 3H), 7.26-7.24 (m, 1H), 7.10 (d, J = 7.5 Hz, 2H), 6.27 (s, 1H), 5.48- |
| | 5.45 (m, 1H), 5.12 (d, J = 16.0 Hz, 1H), 4.57 (d, J = 16.1 Hz, 1H), 4.37-4.33 |
| | (m, 2H), 4.08 (s, 1H), 3.85-3.74 (m, 1H), 3.60-3.56 (m, 2H), 3.49 (p, J = |
| | 6.1 Hz, 1H), 3.35-3.28 (m, 1H), 2.46 (s, 1H), 2.27-2.19 (m, 1H), 2.11 (s, |
| | 6H), 2.03-1.94 (m, 2H), 1.82-1.72 (m, 3H), 1.13 (d, J = 6.0 Hz, 3H), 1.08 |
| | (d, J = 6.1 Hz, 3H). |
| II-10 | ¹H NMR (400 MHz, CDCl₃) δ 8.82-8.78 (m, 1H), 7.88-7.82 (m, 3H), 7.76 |
| | (s, 1H), 7.62 (t, J = 7.7 Hz, 1H), 7.25-7.20 (m, 1H), 7.08 (d, J = 7.6 Hz, 2H), |
| | 6.25 (s, 1H), 5.38-5.30 (m, 1H), 5.20 (d, J = 16.2 Hz, 1H), 4.57 (d, J = 15.9 |
| | Hz, 1H), 4.42-4.32 (m, 2H), 4.27 (t, J = 11.2 Hz, 1H), 3.95-3.85 (m, 1H), |
| | 3.60-3.53 (m, 2H), 3.50-3.34 (m, 2H), 2.76-2.66 (m, 1H), 2.38-2.29 (m, |
| | 1H), 2.11-1.90 (m, 13H), 1.12 (d, J = 6.0 Hz, 3H), 1.06 (d, J = 6.1 Hz, 3H). |
| II-11 | ¹H NMR (400 MHz, CDCl₃) δ 8.77-8.73 (m, 1H), 7.89-7.75 (m, 4H), 7.61 |
| | (t, J = 7.8 Hz, 1H), 7.25-7.20 (m, 1H), 7.09 (d, J = 7.6 Hz, 2H), 6.26 (s, 1H), |
| | 5.42-5.31 (m, 1H), 5.11 (d, J = 15.9 Hz, 1H), 4.50 (d, J = 16.0 Hz, 1H), 4.39 |
| | -4.30 (m, 3H), 3.98-3.91 (m, 1H), 3.59-3.54 (m, 2H), 3.51-3.43 (m, 1H), |
| | 3.40-3.31 (m, 1H), 2.76-2.67 (m, 1H), 2.44-2.36 (m, 1H), 2.11-1.92 (m, |
| | 12H), 1.62-1.59 (m, 1H), 1.12 (d, J = 6.0 Hz, 3H), 1.05 (d, J = 6.1 Hz, 3H). |
| II-12 | ¹H NMR (400 MHz, CDCl₃) δ 8.74 (s, 1H), 7.88-7.81 (m, 3H), 7.76-7.71 |
| | (m, 1H), 7.65-7.58 (m, 1H), 7.24-7.19 (m, 1H), 7.09 (d, J = 7.4 Hz, 2H), |
| | 6.26 (s, 1H), 5.46-5.40 (m, 1H), 5.05 (d, J = 13.8 Hz, 1H), 4.46-4.25 (m, |
| | 6H), 3.59 (s, 2H), 3.51-3.44 (m, 1H), 2.09 (s, 7H), 1.25-1.21 (m, 6H), 1.12 |
| | (d, J = 5.7 Hz, 4H), 1.05 (d, J = 5.9 Hz, 4H). |
| II-13 | ¹H NMR (400 MHz, CDCl₃) δ 8.76 (t, J = 1.8 Hz, 1H), 7.96 (d, J = 7.9 Hz, |
| | 1H), 7.86 (s, 1H), 7.81 (dt, J = 7.7, 1.4 Hz, 1H), 7.69 (s, 1H), 7.63 (t, J = 7.8 |
| | Hz, 1H), 7.23 (t, J = 7.6 Hz, 1H), 7.07 (d, J = 7.7 Hz, 2H), 6.21 (s, 1H), 5.45 |
| | (dd, J = 11.2, 4.2 Hz, 1H), 5.10 (d, J = 16.0 Hz, 1H), 4.37-4.27 (m, 1H), 4.27 |
| | -4.17 (m, 1H), 4.17-4.03 (m, 2H), 3.95-3.84 (m, 1H), 3.76-3.63 (m, 1H), |
| | 3.56-3.45 (m, 1H), 3.18 (td, J = 11.3, 3.3 Hz, 1H), 3.02-2.92 (m, 1H), 2.18- |
| | 2.08 (m, 3H), 2.05 (s, 6H), 1.91-1.80 (m, 2H), 1.76-1.65 (m, 2H), 1.55- |
| | 1.40 (m, 2H), 1.39-1.30 (m, 1H), 1.29 (d, J = 6.3 Hz, 3H), 1.26-1.18 (m, |
| | 1H), 1.16-1.03 (m, 1H). |
| II-14 | ¹H NMR (400 MHz, CDCl₃) δ 8.68 (t, J = 1.9 Hz, 1H), 7.98 (d, J = 7.9 Hz, |
| | 1H), 7.96-7.81 (m, 2H), 7.80 (dt, J = 7.7, 1.4 Hz, 1H), 7.64 (t, J = 7.8 Hz, |
| | 1H), 7.23 (t, J = 7.6 Hz, 1H), 7.07 (d, J = 7.6 Hz, 2H), 6.22 (s, 1H), 5.36 (dd, |
| | J = 11.0, 4.3 Hz, 1H), 5.06 (d, J = 16.0 Hz, 1H), 4.62 (s, 1H), 4.40-4.28 (m, |
| | 1H), 4.28-4.19 (m, 1H), 4.17 (d, J = 16.1 Hz, 1H), 4.06 (t, J = 11.4 Hz, 1H), |
| | 3.92-3.82 (m, 1H), 3.21-3.04 (m, 2H), 3.00-2.88 (m, 1H), 2.05 (s, 6H), |
| | 1.97-1.87 (m, 1H), 1.87-1.79 (m, 1H), 1.79-1.61 (m, 6H), 1.62-1.39 (m, |
| | 3H), 1.35 (dt, J = 12.8, 4.1 Hz, 1H), 1.29 (d, J = 6.8 Hz, 3H), 1.26-1.20 (m, |
| | 1H), 1.15-1.03 (m, 1H). |
| II-15 | ¹H NMR (400 MHz, Chloroform-d) δ 8.64 (s, 1H), 8.15 (d, J = 7.9 Hz, 1H), |
| | 8.01 (s, 1H), 7.92 (s, 1H), 7.82 (d, J = 7.7, 1.3 Hz, 1H), 7.67 (t, J = 7.8 Hz, |
| | 1H), 7.19 (t, J = 7.6 Hz, 1H), 7.01 (d, J = 7.6 Hz, 2H), 6.18 (s, 1H), 5.43 (dd, |
| | J = 11.2, 4.0 Hz, 1H), 5.25-5.14 (m, 1H), 5.11 (d, J = 15.1 Hz, 1H), 4.17 (t, J = |
| | 11.4 Hz, 1H), 4.06-3.93 (m, 2H), 3.13 (s, 3H), 2.90-2.77 (m, 1H), 2.69- |
| | 2.52 (m, 4H), 1.99 (s, 6H), 1.63 (q, J = 7.9 Hz, 3H), 1.21-1.12 (m, 1H), 0.83 |
| | (s, 9H). |
| II-18 | ¹H NMR (400 MHz, CDCl₃) δ 8.81 (t, J = 1.8 Hz, 1H), 8.07 (dt, J = 8.1, 1.4 |
| | Hz, 1H), 7.82 (s, 1H), 7.77 (dd, J = 7.7, 1.7 Hz, 1H), 7.62 (t, J = 7.7 Hz, 1H), |
| | 7.26 (t, J = 7.6 Hz, 1H), 7.11 (d, J = 7.7 Hz, 2H), 6.08 (s, 1H), 5.61 (dd, J = |
| | 10.7, 3.9 Hz, 1H), 5.01 (d, J = 15.6 Hz, 1H), 4.28-4.04 (m, 3H), 3.30 (d, J = |
| | 14.9 Hz, 1H), 3.14 (d, 1H), 3.11 (s, 3H), 2.24-2.15 (m, 4H), 2.14-2.05 (m, |
| | 6H), 1.96-1.86 (m, 1H), 1.84-1.74 (m, 1H), 1.69 (dd, J = 15.1, 8.5 Hz, 1H), |
| | 1.55 (d, J = 15.0 Hz, 1H), 0.64 (s, 9H). |
| II-21 | ¹H NMR (400 MHz, CDCl₃) δ 8.70 (s, 1H), 8.23 (d, J = 7.9 Hz, 1H), 8.18 (s, |
| | 1H), 8.05 (s, 1H), 7.87-7.82 (m, 1H), 7.71 (t, J = 7.8 Hz, 1H), 7.20 (t, J = 7.6 |
| | Hz, 1H), 7.03 (d, J = 7.6 Hz, 2H), 6.18 (s, 1H), 5.37 (dd, J = 10.8, 3.9 Hz, |
| | 1H), 5.24-5.12 (m, 2H), 4.76-4.69 (m, 1H), 4.14-4.03 (m, 2H), 4.00-3.86 |
| | (m, 2H), 3.37-3.29 (m, 1H), 3.22-3.06 (m, 3H), 2.00 (s, 6H), 1.66-1.53 (m, |
| | 7H), 0.83 (s, 9H). |
| II-23 | ¹H NMR (400 MHz, CDCl₃) δ 8.72 (t, J = 1.8 Hz, 1H), 7.90 (s, 1H), 7.83 (s, |
| | 1H), 7.76 (dt, J = 7.6, 1.4 Hz, 1H), 7.47 (t, J = 7.8 Hz, 1H), 7.34 (s, 1H), 7.29 |
| | (t, J = 7.6 Hz, 1H), 7.11 (d, J = 7.6 Hz, 2H), 6.21 (s, 1H), 5.47 (dd, J = 11.0, |
| | 4.0 Hz, 1H), 5.27 (d, J = 15.7 Hz, 1H), 4.53-4.34 (m, 2H), 4.10 (d, J = 15.7 |
| | Hz, 1H), 4.03 (t, J = 11.4 Hz, 1H), 3.78 (d, J = 11.1 Hz, 1H), 3.44-3.32 (m, |
| | 1H), 3.20 (s, 4H), 2.35-2.27 (m, 2H), 2.27-2.18 (m, 2H), 2.14 (s, 6H), 1.85- |
| | 1.68 (m, 5H), 1.63-1.49 (m, 3H), 1.48-1.38 (m, 1H), 1.20 (p, J = 10.4 Hz, |
| | 1H). |
| II-24 | ¹H NMR (400 MHz, CDCl₃) δ 8.74 (s, 1H), 7.90 (s, 1H), 7.81 (dt, J = 7.5, 1.5 |
| | Hz, 1H), 7.74-7.60 (m, 2H), 7.57 (t, J = 7.7 Hz, 1H), 7.30 (t, J = 7.6 Hz, 1H), |
| | 7.13 (d, J = 7.6 Hz, 2H), 6.31 (s, 1H), 5.65-5.51 (m, 1H), 5.24 (d, J = 15.9 |
| | Hz, 1H), 4.53-4.41 (m, 1H), 4.33 (d, J = 16.1 Hz, 1H), 4.18 (t, J = 11.4 Hz, |
| | 2H), 3.78-3.63 (m, 2H), 3.57 (s, 1H), 3.34 (t, J = 11.5 Hz, 1H), 3.18 (t, J = |
| | 10.7 Hz, 1H), 2.19 (s, 6H), 2.18-2.09 (m, 3H), 1.96-1.68 (m, 4H), 1.61 (d, J = |
| | 13.9 Hz, 1H), 1.56-1.47 (m, 2H), 1.47-1.37 (m, 1H), 1.29 (d, J = 6.2 Hz, |
| | 3H), 1.22-1.10 (m, 1H). |
| II-25 | ¹H NMR (400 MHz, CDCl₃) δ 8.66 (t, J = 1.7 Hz, 1H), 7.97 (s, 1H), 7.86 (s, |
| | 1H), 7.77 (dt, J = 7.6, 1.4 Hz, 1H), 7.46 (t, J = 7.7 Hz, 1H), 7.34 (d, J = 8.0 |
| | Hz, 1H), 7.28 (t, J = 7.6 Hz, 1H), 7.11 (d, J = 7.6 Hz, 2H), 6.21 (s, 1H), 5.40 |
| | (dd, J = 11.2, 4.1 Hz, 1H), 5.22 (d, J = 15.3 Hz, 1H), 4.70-4.57 (m, 1H), 4.46- |
| | 4.32 (m, 1H), 4.25 (d, J = 13.4 Hz, 1H), 4.04 (t, J = 11.5 Hz, 1H), 3.98 (d, J = |
| | 15.3 Hz, 1H), 3.82-3.70 (m, 1H), 3.44-3.31 (m, 1H), 3.23-3.12 (m, 1H), |
| | 3.00 (td, J = 13.0, 3.2 Hz, 1H), 2.14 (s, 6H), 1.88-1.72 (m, 5H), 1.72-1.61 |
| | (m, 3H), 1.61-1.47 (m, 4H), 1.47-1.34 (m, 1H), 1.23 (d, J = 6.8 Hz, 3H), |
| | 1.20-1.10 (m, 1H). |
| II-26 | ¹H NMR (400 MHz, CDCl₃) δ 8.75 (t, J = 1.8 Hz, 1H), 8.02 (d, J = 7.9 Hz, |
| | 1H), 7.90 (s, 1H), 7.87 (s, 1H), 7.82 (dt, J = 7.7, 1.4 Hz, 1H), 7.63 (t, J = 7.8 |
| | Hz, 1H), 7.21 (t, J = 7.6 Hz, 1H), 7.04 (d, J = 7.6 Hz, 2H), 6.16 (s, 1H), 5.38 |
| | (dd, J = 11.1, 4.2 Hz, 1H), 5.21 (d, J = 15.8 Hz, 1H), 4.61-4.48 (m, 1H), 4.31- |
| | 4.18 (m, 1H), 4.13-4.01 (m, 2H), 3.77-3.67 (m, 1H), 3.65-3.52 (m, 2H), |
| | 3.14 (s, 3H), 2.33-2.26 (m, 2H), 2.25-2.14 (m, 2H), 2.10-2.02 (m, 1H), |
| | 2.01 (s, 6H), 1.82-1.65 (m, 6H), 1.15-1.03 (m, 1H). |
| II-27 | ¹H NMR (400 MHz, CDCl₃) δ 8.73 (t, J = 1.8 Hz, 1H), 7.92 (s, 1H), 7.84- |
| | 7.76 (m, 2H), 7.72 (d, J = 7.9 Hz, 1H), 7.57 (t, J = 7.8 Hz, 1H), 7.25 (t, J = |
| | 7.6 Hz, 1H), 7.10 (d, J = 7.6 Hz, 2H), 6.22 (s, 1H), 5.48 (dd, J = 11.0, 4.1 Hz, |
| | 1H), 5.21 (d, J = 15.9 Hz, 1H), 4.54-4.42 (m, 1H), 4.42-4.30 (m, 1H), 4.20 |
| | (d, J = 15.9 Hz, 1H), 4.06 (t, J = 11.4 Hz, 1H), 3.66-3.60 (m, 1H), 3.61- |
| | 3.56 (m, 1H), 3.56-3.50 (m, 1H), 3.23 (s, 3H), 2.41-2.29 (m, 2H), 2.28- |
| | 2.17 (m, 2H), 2.10 (s, 6H), 2.01-1.90 (m, 2H), 1.90-1.69 (m, 5H), 1.41- |
| | 1.28 (m, 1H). |
| II-28 | ¹H NMR (400 MHz, CDCl₃) δ 8.77-8.74 (m, 1H), 8.05 (d, J = 7.9 Hz, 1H), |
| | 7.91 (d, J = 5.7 Hz, 2H), 7.86 (d, J = 1.4 Hz, 1H), 7.66 (t, J = 7.8 Hz, 1H), |
| | 7.20 (t, J = 7.6 Hz, 1H), 7.04 (d, J = 7.6 Hz, 2H), 6.19 (s, 1H), 5.37 (d, J = 7.2 |
| | Hz, 1H), 5.30 (s, 1H), 5.21 (d, J = 15.5 Hz, 1H), 4.61-4.51 (m, 1H), 4.16- |
| | 3.97 (m, 4H), 3.13 (s, 3H), 2.33-2.16 (m, 5H), 2.01 (s, 6H), 1.93-1.81 (m, |
| | 2H), 1.44-1.28 (m, 3H), 1.00 (s, 3H), 0.97 (d, J = 6.6 Hz, 1H), 0.91-0.83 |
| | (m, 3H). |
| II-29 | ¹H NMR (400 MHz, CDCl₃) δ 8.83 (t, J = 1.8 Hz, 1H), 8.08 (d, J = 7.9 Hz, |
| | 1H), 7.89 (s, 1H), 7.83-7.76 (m, 1H), 7.69-7.66 (m, 1H), 7.66-7.62 (m, |
| | 1H), 7.24 (t, J = 7.6 Hz, 1H), 7.08 (d, J = 7.6 Hz, 2H), 6.27 (s, 1H), 5.59- |
| | 5.45 (m, 1H), 5.20 (d, J = 16.2 Hz, 1H), 4.41-4.19 (m, 3H), 4.06 (t, J = 11.3 |
| | Hz, 1H), 3.95-3.86 (m, 1H), 3.81-3.69 (m, 2H), 3.57-3.46 (m, 1H), 3.38- |
| | 3.29 (m, 1H), 3.18 (td, J = 11.1, 3.6 Hz, 1H), 2.24-2.13 (m, 3H), 2.06 (s, |
| | 6H), 1.91-1.82 (m, 1H), 1.74 (t, J = 12.7 Hz, 1H), 1.54-1.43 (m, 2H), 1.42- |
| | 1.25 (m, 2H), 1.21 (d, J = 6.3 Hz, 3H), 0.85-0.70 (m, 2H). |
| II-30 | ¹H NMR (400 MHz, CDCl₃) δ 8.75 (t, J = 1.8 Hz, 1H), 8.01 (d, J = 7.9 Hz, |
| | 1H), 7.99-7.82 (m, 2H), 7.79 (dt, J = 7.8, 1.4 Hz, 1H), 7.63 (t, J = 7.8 Hz, |
| | 1H), 7.26-7.21 (m, 1H), 7.08 (d, J = 7.6 Hz, 2H), 6.23 (s, 1H), 5.44 (dd, J = |
| | 10.8, 4.1 Hz, 1H), 5.15 (d, J = 16.1 Hz, 1H), 4.64 (s, 1H), 4.36 (d, J = 16.5 |
| | Hz, 1H), 4.32-4.16 (m, 2H), 4.09 (t, J = 11.2 Hz, 1H), 3.95-3.83 (m, 1H), |
| | 3.77-3.66 (m, 1H), 3.33 (td, J = 11.6, 2.1 Hz, 1H), 3.26-3.09 (m, 2H), 2.06 |
| | (s, 6H), 1.98-1.87 (m, 1H), 1.86-1.63 (m, 5H), 1.63-1.41 (m, 3H), 1.41- |
| | 1.32 (m, 1H), 1.28 (td, J = 11.7, 3.9 Hz, 1H), 1.21 (d, J = 6.8 Hz, 3H), 0.83- |
| | 0.66 (m, 2H). |
| II-31 | ¹H NMR (400 MHz, CDCl₃) δ 8.79 (t, J = 1.8 Hz, 1H), 8.06 (d, J = 7.9 Hz, |
| | 1H), 7.99 (s, 1H), 7.85-7.75 (m, 2H), 7.64 (t, J = 7.8 Hz, 1H), 7.26-7.20 (m, |
| | 1H), 7.07 (d, J = 7.6 Hz, 2H), 6.23 (s, 1H), 5.47 (dd, J = 10.8, 4.1 Hz, 1H), |
| | 5.20 (d, J = 16.0 Hz, 1H), 4.57-4.40 (m, 1H), 4.34 (d, J = 16.4 Hz, 1H), 4.30- |
| | 4.19 (m, 1H), 4.09 (t, J = 11.2 Hz, 1H), 3.94-3.85 (m, 1H), 3.79-3.68 (m, |
| | 1H), 3.37-3.28 (m, 1H), 3.24 (s, 3H), 3.18 (td, J = 11.3, 3.4 Hz, 1H), 2.41- |
| | 2.29 (m, 2H), 2.29-2.19 (m, 2H), 2.05 (s, 6H), 1.87-1.69 (m, 3H), 1.56- |
| | 1.43 (m, 2H), 1.43-1.34 (m, 1H), 1.28 (qd, J = 11.5, 4.2 Hz, 1H), 0.85-0.70 |
| | (m, 2H). |
| II-32 | ¹H NMR (400 MHz, Chloroform-d) δ 9.28 (t, J = 1.8 Hz, 1H), 8.87 (s, 1H), |
| | 8.38-8.27 (m, 2H), 8.11 (dt, J = 7.8, 1.4 Hz, 1H), 8.05 (s, 1H), 7.75 (t, J = |
| | 7.8 Hz, 1H), 7.23 (t, J = 7.7 Hz, 1H), 7.07 (d, J = 7.6 Hz, 2H), 6.22 (s, 1H), |
| | 5.48 (dd, J = 10.5, 3.3 Hz, 1H), 4.90 (t, J = 10.7 Hz, 1H), 4.52-4.39 (m, 1H), |
| | 3.80-3.71 (m, 1H), 2.74 (s, 3H), 2.54 (dd, J = 15.5, 6.0 Hz, 1H), 2.16-2.04 |
| | (m, 2H), 2.02 (d, J = 5.6 Hz, 6H), 2.00-1.95 (m, 1H), 1.95-1.88 (m, 1H), |
| | 1.58-1.53 (m, 2H), 1.39-1.29 (m, 1H), 0.64 (s, 9H). |
| II-33 | ¹H NMR (400 MHz, Chloroform-d) δ 8.70 (s, 1H), 8.15 (d, J = 7.9 Hz, 1H), |
| | 8.03 (s, 1H), 7.96 (s, 1H), 7.85 (d, J = 7.7, 1.4 Hz, 1H), 7.69 (t, J = 7.8 Hz, |
| | 1H), 7.19 (t, J = 7.6 Hz, 1H), 7.02 (d, J = 7.6 Hz, 2H), 6.18 (s, 1H), 5.39- |
| | 5.30 (m, 1H), 5.21 (d, J = 15.5 Hz, 1H), 4.79-4.68 (m, 1H), 4.07-3.97 (m, |
| | 3H), 3.06-2.94 (m, 2H), 2.85-2.66 (m, 2H), 1.99 (s, 6H), 1.64-1.59 (m, |
| | 3H), 1.20-1.10 (m, 1H), 0.82 (s, 9H). |
| II-34 | ¹H NMR (400 MHz, Chloroform-d) δ 8.75 (s, 1H), 8.10 (d, J = 7.9 Hz, 1H), |
| | 7.93 (d, J = 4.8 Hz, 1H), 7.84 (d, J = 7.6 Hz, 1H), 7.67 (t, J = 7.8 Hz, 1H), |
| | 7.20 (t, J = 7.6 Hz, 1H), 7.03 (d, J = 7.6 Hz, 2H), 6.18 (s, 1H), 5.40 (d, J = 8.3 |
| | Hz, 1H), 5.19 (d, J = 15.4 Hz, 1H), 4.91 (q, J = 8.1 Hz, 1H), 4.12-3.96 (m, |
| | 3H), 2.51 (td, J = 11.9, 8.3 Hz, 2H), 2.31-2.24 (m, 2H), 2.01 (s, 6H), 1.63- |
| | 1.59 (m, 2H), 1.16 (td, J = 12.5, 12.1, 5.4 Hz, 1H), 0.82 (s, 11H), 0.51-0.41 |
| | (m, 4H). |
| II-35 | ¹H NMR (400 MHz, Chloroform-d) δ 9.49-8.76 (m, 2H), 8.07 (d, J = 7.8 Hz, |
| | 1H), 7.91 (dd, J = 7.8, 2.8 Hz, 3H), 7.66 (t, J = 7.8 Hz, 1H), 7.24-7.17 (m, |
| | 1H), 7.06 (d, J = 7.6 Hz, 2H), 6.21 (s, 1H), 5.42 (dd, J = 10.9, 3.6 Hz, 1H), |
| | 5.16 (d, J = 15.6 Hz, 1H), 4.60-4.47 (m, 1H), 4.12-4.02 (m, 1H), 4.02-3.90 |
| | (m, 2H), 3.13 (s, 3H), 2.43 (s, 1H), 2.33-2.25 (m, 2H), 2.25-2.15 (m, 2H), |
| | 2.04 (s, 6H), 1.96-1.87 (m, 1H), 1.79-1.72 (m, 2H), 1.72-1.68 (m, 1H), |
| | 1.58-1.50 (m, 6H). |
| II-36 | ¹H NMR (400 MHz, Chloroform-d) δ 8.94 (s, 1H), 8.49 (s, 1H), 8.19 (s, 1H), |
| | 8.03-7.93 (m, 2H), 7.53 (t, J = 7.7 Hz, 1H), 7.39-7.33 (m, 1H), 7.23-7.15 |
| | (m, 1H), 7.05 (d, J = 7.6 Hz, 2H), 6.16 (s, 1H), 5.69 (dd, J = 11.2, 2.0 Hz, |
| | 1H), 5.26 (d, J = 17.1 Hz, 1H), 5.07 (dd, J = 11.2, 9.4 Hz, 1H), 4.58-4.45 (m, |
| | 1H), 4.37 (d, J = 17.1 Hz, 1H), 2.90-2.83 (m, 1H), 2.80 (s, 3H), 2.31 (dd, J = |
| | 15.2, 5.5 Hz, 1H), 2.22 (dd, J = 15.2, 4.7 Hz, 1H), 2.19-2.13 (m, 1H), 2.11- |
| | 2.04 (m, 1H), 2.03 (s, 6H), 2.01-1.93 (m, 1H), 1.72-1.60 (m, 2H), 1.54- |
| | 1.51 (m, 1H), 0.90 (s, 9H). |
| II-38 | ¹H NMR (400 MHz, CDCl₃) δ 8.92-8.73 (m, 1H), 8.11-8.05 (m, 1H), 7.91 |
| | (d, J = 11.0 Hz, 1H), 7.83 (d, J = 7.6 Hz, 1H), 7.75 (d, J = 5.9 Hz, 1H), 7.65- |
| | 7.53 (m, 1H), 7.22-7.14 (m, 1H), 7.07-6.99 (m, 2H), 6.22-6.16 (m, 1H), |
| | 5.52-5.33 (m, 1H), 5.07 (dd, J = 15.5, 7.5 Hz, 1H), 4.83 (td, J = 15.1, 8.2 Hz, |
| | 1H), 4.30-4.21 (m, 1H), 4.15-3.95 (m, 2H), 3.66-3.53 (m, 1H), 3.39-3.20 |
| | (m, 1H), 2.49-2.40 (m, 2H), 2.02 (br. s., 6H), 1.96-1.82 (m, 4H), 1.77-1.67 |
| | (m, 2H), 1.53 (d, J = 14.7 Hz, 1H), 1.44-1.33 (m, 4H), 0.69-0.52 (m, 9H), |
| | (one exchangeable proton is missing). |
| III-4 | ¹H NMR (400 MHz, Chloroform-d) δ 8.56 (s, 1H), 8.16-7.98 (m, 3H), 7.83 |
| | (d, J = 7.7 Hz, 1H), 7.66 (t, J = 7.8 Hz, 1H), 7.25-7.18 (m, 2H), 7.06 (d, J = |
| | 7.6 Hz, 2H), 6.21 (s, 1H), 5.45-5.24 (m, 2H), 5.21 (d, J = 15.0 Hz, 1H), 5.07- |
| | 4.95 (m, 1H), 4.21-4.12 (m, 1H), 3.00-2.87 (m, 1H), 2.01 (s, 6H), 1.73 (d, |
| | J = 15.9 Hz, 1H), 1.60-1.54 (m, 3H), 1.50-1.38 (m, 2H), 0.98-0.86 (m, |
| | 2H), 0.61 (s, 9H). |
| III-6 | ¹H NMR (400 MHz, CDCl₃) δ 8.89 (t, J = 1.6 Hz, 1H), 8.15 (dt, J = 8.1, 1.0 |
| | Hz, 1H), 7.88 (dt, J = 7.8, 1.0 Hz, 1H), 7.69 (t, J = 7.8 Hz, 1H), 7.57-7.52 |
| | (m, 2H), 7.26-7.18 (m, 2H), 7.06 (d, J = 7.6 Hz, 2H), 6.25 (s, 1H), 5.48- |
| | 5.37 (m, 2H), 4.57 (d, J = 16.6 Hz, 1H), 4.31-4.19 (m, 1H), 3.99 (t, J = 11.4 |
| | Hz, 1H), 1.99 (s, 6H), 1.80 (dd, J = 15.2, 8.8 Hz, 1H), 1.58 (d, J = 13.7 Hz, |
| | 1H, overlapped with water), 0.64 (s, 9H); one labile proton missing. |
| III-7 | ¹H NMR (400 MHz, CDCl₃) δ 8.92 (s, 1H), 8.17-8.05 (m, 1H), 7.82 (br. s., |
| | 1H), 7.59 (br. s., 1H), 7.39 (d, J = 8.3 Hz, 1H), 7.31-7.27 (m, 1H, overlapped |
| | with CHC13), 7.23-7.11 (m, 2H), 7.09-6.97 (m, 2H), 6.21 (br. s., 1H), 5.52 |
| | (dd, J = 10.9, 4.0 Hz, 1H), 5.38 (d, J = 16.1 Hz, 1H), 4.50 (d, J = 16.9 Hz, |
| | 1H), 4.29-4.15 (m, 1H), 4.05-3.90 (m, 1H), 3.58 (dt, J = 13.9, 7.1 Hz, 1H), |
| | 1.99 (br. s., 6H), 1.78 (dd, J = 14.9, 9.0 Hz, 1H), 1.61 (s, 1H), 1.42 (s, 3H), |
| | 1.40 (s, 3H), 0.64 (s, 9H); one labile proton missing. |
| III-8 | ¹H NMR (400 MHz, CDCl₃) δ 8.94 (s, 1H), 8.37 (br. s., 1H), 8.13 (d, J = 8.1 |
| | Hz, 1H), 7.88 (d, J = 7.6 Hz, 1H), 7.68 (t, J = 7.8 Hz, 1H), 7.47 (dd, J = 7.6, |
| | 1.7 Hz, 1H), 7.38-7.29 (m, 2H), 7.21 (t, J = 7.6 Hz, 1H), 7.07 (d, J = 7.8 Hz, |
| | 2H), 6.26 (s, 1H), 6.06 (d, J = 0.7 Hz, 1H), 5.53 (dd, J = 11.2, 4.4 Hz, 1H), |
| | 5.47 (t, J = 1.5 Hz, 1H), 5.42 (d, J = 16.6 Hz, 1H), 4.49 (d, J = 16.6 Hz, 1H), |
| | 4.31-4.21 (m, 1H), 3.99 (t, J = 11.5 Hz, 1H), 2.35 (s, 3H), 2.01 (s, 6H), 1.74 |
| | (dd, J = 15.3, 8.9 Hz, 1H), 1.61-1.59 (m., 1H, overlapped with water), 0.64 |
| | (s, 9H). |
| III-9 | ¹H NMR (400 MHz, Methanol-d4) δ 9.01 (dd, J = 6.8, 1.0 Hz, 1H), 8.78-8.64 |
| | (m, 2H), 8.48 (s, 1H), 8.09 (dt, J = 7.9, 1.4 Hz, 1H), 7.82 (dt, J = 7.8, 1.4 Hz, |
| | 1H), 7.74 (t, J = 7.7 Hz, 1H), 7.67 (dd, J = 7.6, 6.7 Hz, 1H), 7.28 (t, J = 7.6 |
| | Hz, 1H), 7.15 (d, J = 7.7 Hz, 2H), 6.34 (s, 1H), 5.41 (dd, J = 10.4, 4.2 Hz, |
| | 1H), 5.21 (dd, J = 16.1, 0.9 Hz, 1H), 4.90 (d, J = 16.3 Hz, 1H), 4.37 (dd, J = |
| | 11.8, 10.4 Hz, 1H), 4.31-4.21 (m, 1H), 2.81 (s, 3H), 2.25-1.94 (m, 6H), 1.88 |
| | (dd, J = 15.5, 8.8 Hz, 1H), 1.67-1.58 (m, 1H), 0.61 (s, 9H). |
| III-10 | ¹H NMR (400 MHz, CDCl₃) δ 8.52 (t, J = 1.8 Hz, 1H), 8.35 (dd, J = 6.9, 1.1 |
| | Hz, 1H), 8.13-8.08 (m, 1H), 7.98 (s, 1H), 7.78 (dt, J = 7.8, 1.5 Hz, 1H), 7.70- |
| | 7.60 (m, 2H), 7.21 (t, J = 7.6 Hz, 1H), 7.04 (d, J = 7.6 Hz, 2H), 6.94 (t, J = |
| | 7.0 Hz, 1H), 6.22 (s, 1H), 5.22 (d, J = 14.8 Hz, 1H), 5.05 (dd, J = 11.3, 4.5 |
| | Hz, 1H), 4.59 (t, J = 11.5 Hz, 1H), 4.50 (d, J = 14.8 Hz, 1H), 4.16-4.05 (m, |
| | 1H), 2.00 (s, 7H), 1.60-1.51 (m, 1H), 0.59 (s, 9H). |
| III-11 | ¹H NMR (400 MHz, Chloroform-d) δ 9.98 (s, 1H), 8.70-8.65 (m, 1H), 8.22 |
| | (d, J = 7.9 Hz, 1H), 7.88 (d, J = 2.3 Hz, 1H), 7.87-7.76 (m, 2H), 7.70 (t, J = |
| | 7.8 Hz, 1H), 7.51 (d, J = 8.5 Hz, 1H), 7.17 (t, J = 7.6 Hz, 1H), 7.03-6.95 (m, |
| | 3H), 6.13 (s, 1H), 5.53 (d, J = 15.1 Hz, 1H), 5.18 (dd, J = 11.1, 3.9 Hz, 1H), |
| | 4.28 (d, J = 15.1 Hz, 1H), 4.14 (t, J = 11.4 Hz, 1H), 4.08-3.97 (m, 1H), 1.94 |
| | (s, 6H), 1.82-1.73 (m, 1H), 1.31-1.21 (m, 1H), 1.21-1.12 (m, 1H), 0.82 (s, |
| | 9H), 0.81-0.76 (m, 1H). |
| III-12 | ¹H NMR (400 MHz, Chloroform-d) δ 8.53 (d, J = 1.8 Hz, 1H), 8.18 (d, J = |
| | 7.8 Hz, 1H), 7.92-7.85 (m, 2H), 7.71 (t, J = 7.8 Hz, 1H), 7.66 (d, J = 6.5 Hz, |
| | 1H), 7.27-7.23 (m, 1H), 7.12 (dd, J = 7.9, 6.5 Hz, 1H), 7.09 (d, J = 7.6 Hz, |
| | 2H), 6.83 (d, J = 7.8 Hz, 1H), 6.33 (s, 1H), 5.27 (t, J = 11.2 Hz, 1H), 5.20 (d, |
| | J = 15.0 Hz, 1H), 5.12 (dd, J = 10.7, 4.3 Hz, 1H), 4.62 (d, J = 15.1 Hz, 1H), |
| | 4.23-4.11 (m, 1H), 2.06 (s, 6H), 1.89 (dd, J = 15.6, 8.9 Hz, 1H), 1.69 (d, J = |
| | 15.5 Hz, 1H), 0.61 (s, 9H). |
| III-15 | ¹H NMR (400 MHz, CDCl₃) δ 8.61 (s, 1H), 8.09 (d, J = 7.9 Hz, 1H), 7.82 (d, |
| | J = 7.6 Hz, 1H), 7.78 (s, 1H), 7.72 (d, J = 6.6 Hz, 1H), 7.63 (t, J = 7.8 Hz, |
| | 1H), 7.18 (t, J = 7.6 Hz, 1H), 7.01 (d, J = 7.6 Hz, 2H), 6.77 (t, J = 7.1 Hz, |
| | 1H), 6.71-6.63 (m, 1H), 6.13 (s, 1H), 5.21 (d, J = 15.1 Hz, 1H), 5.07-4.97 |
| | (m, 1H), 4.67 (d, J = 15.2 Hz, 1H), 4.32 (t, J = 11.3 Hz, 1H), 4.16-4.04 (m, |
| | 1H), 2.10-1.96 (m, 2H), 1.94 (s, 6H), 1.46-1.38 (m, 1H), 0.53 (s, 9H). |
| III-17 | ¹H NMR (400 MHz, CDCl₃) δ 8.67 (t, J = 1.8 Hz, 1H), 8.36 (s, 1H), 8.04 (dt, |
| | J = 8.1, 1.3 Hz, 1H), 7.96 (dt, J = 8.2, 1.0 Hz, 1H), 7.89 (dt, J = 7.7, 1.3 Hz, |
| | 1H), 7.64 (t, J = 7.8 Hz, 1H), 7.41 (dt, J = 8.6, 1.1 Hz, 1H), 7.39-7.33 (m, |
| | 1H), 7.19 (t, J = 7.6 Hz, 1H), 7.15 (ddd, J = 7.9, 6.5, 1.1 Hz, 1H), 7.05 (d, J = |
| | 7.6 Hz, 2H), 6.20 (s, 1H), 5.73 (d, J = 15.3 Hz, 1H), 5.14-5.02 (m, 1H), 4.83 |
| | (hept, J = 6.7 Hz, 1H), 4.54 (d, J = 15.3 Hz, 1H), 4.23-4.08 (m, 2H), 2.10- |
| | 2.00 (m, 1H), 1.98 (s, 6H), 1.62 (d, J = 6.5 Hz, 3H), 1.61 (d, J = 6.5 Hz, 3H), |
| | 1.58-1.55 (m, 1H), 0.65 (s, 9H). |
| III-18 | ¹H NMR (400 MHz, CDCl₃) δ 8.94 (s, 1H), 8.80 (t, J = 1.8 Hz, 1H), 8.05- |
| | 7.99 (m, 1H), 7.83 (dt, J = 7.7, 1.4 Hz, 1H), 7.62 (t, J = 7.8 Hz, 1H), 7.22 (t, J = |
| | 7.6 Hz, 1H), 7.06 (d, J = 7.7 Hz, 2H), 6.23 (s, 1H), 5.53 (dd, J = 11.3, 4.3 |
| | Hz, 1H), 5.26 (d, J = 15.5 Hz, 1H), 4.88-4.77 (m, 1H), 4.48 (s, 2H), 4.46- |
| | 4.36 (m, 1H), 4.19 (d, J = 15.5 Hz, 1H), 4.10 (t, J = 11.5 Hz, 1H), 2.03-1.98 |
| | (m, 6H), 1.88-1.81 (m, 1H), 1.41 (dd, J = 15.2, 10.2 Hz, 1H), 1.35 (d, J = 4.0 |
| | Hz, 3H), 1.33 (d, J = 4.1 Hz, 3H), 0.53 (s, 3H), 0.38-0.32 (m, 1H), 0.32- |
| | 0.24 (m, 1H), 0.20-0.13 (m, 1H), 0.06-0.01 (m, 1H). |
| III-19 | ¹H NMR (400 MHz, CDCl₃) δ 8.95 (s, 1H), 8.71 (t, J = 1.8 Hz, 1H), 8.06 |
| | (d, J = 7.9 Hz, 1H), 7.78 (dt, J = 7.7, 1.4 Hz, 1H), 7.65 (t, J = 7.8 Hz, 1H), 7.22 (t, |
| | J = 7.6 Hz, 1H), 7.03 (d, J = 7.6 Hz, 2H), 6.20 (s, 1H), 5.51 (dd, J = 10.6, 3.4 |
| | Hz, 1H), 5.25 (d, J = 15.5 Hz, 1H), 4.83 (hept, J = 6.8 Hz, 1H), 4.49-4.43 |
| | (m, 2H), 4.29 (d, J = 15.5 Hz, 1H), 4.20-4.03 (m, 2H), 2.02-1.97 (m, 7H), |
| | 1.81-1.73 (m, 2H), 1.35 (d, J = 4.7 Hz, 3H), 1.33 (d, J = 4.6 Hz, 3H), 0.96- |
| | 0.87 (m, 4H), 0.23-0.16 (m, 2H), 0.16-0.10 (m, 2H). |
| III-22 | ¹H NMR (400 MHz, CDCl₃) δ 9.05 (s, 1H), 8.44 (t, J = 1.8 Hz, 1H), 8.26 |
| | (d, J = 7.9 Hz, 1H), 7.78 (dt, J = 7.7, 1.4 Hz, 1H), 7.68 (t, J = 7.8 Hz, 1H), 7.18 (t, |
| | J = 7.6 Hz, 1H), 6.98 (d, J = 7.7 Hz, 2H), 6.12 (s, 1H), 5.39 (d, J = 14.5 Hz, |
| | 1H), 5.10 (dd, J = 11.2, 4.2 Hz, 1H), 4.84 (hept, J = 6.8 Hz, 1H), 4.53-4.42 |
| | (m, 3H), 4.36 (d, J = 14.5 Hz, 1H), 4.09-3.98 (m, 1H), 2.00-1.79 (m, 6H), |
| | 1.79-1.66 (m, 2H), 1.38 (d, J = 2.6 Hz, 3H), 1.36 (d, J = 2.7 Hz, 3H), 1.19- |
| | 1.07 (m, 1H), 0.92 (s, 3H), 0.90-0.80 (m, 1H), 0.22-0.10 (m, 4H). |
| III-23 | ¹H NMR (400 MHz, CDCl₃) δ 8.89 (m, 2H), 8.14-8.09 (m, 1H), 7.87-7.82 |
| | (m, 1H), 7.68 (t, J = 7.7 Hz, 1H), 7.23-7.19 (overlapped with CDCl₃, m, 1H), |
| | 7.12-7.06 (m, 2H), 6.30 (s, 1H), 5.68 (dd, J = 11.0, 4.2 Hz, 1H), 5.24 (d, J = |
| | 16.1 Hz, 1H), 4.79-4.71 (m, 1H), 4.60-4.50 (m, 1H), 4.36 (d, J = 15.9 Hz, |
| | 1H), 4.26-4.17 (m, 1H), 4.06 (t, J = 11.5 Hz, 1H), 2.04 (br. s, 6H), 1.73- |
| | 1.61 (overlapped with water, m, 5H), 1.47 (d, J = 7.1 Hz, 3H), 1.44 (d, J = 6.8 |
| | Hz, 3H), 0.68 (s, 9H). (1H missing, labile H). |
| III-24 | ¹H NMR (400 MHz, CDCl₃) δ 8.91 (s, 1H), 8.91-8.88 (m, 1H), 8.15-8.07 |
| | (m, 1H), 7.93-7.80 (m, 1H), 7.73-7.62 (m, 1H), 7.26-7.16 (m, 1H), 7.13- |
| | 7.05 (m, 2H), 6.30 (s, 1H), 5.50 (dd, J = 11.1, 4.3 Hz, 1H), 5.29 (d, J = 15.9 |
| | Hz, 1H), 4.73 (q, J = 6.8 Hz, 1H), 4.58-4.49 (m, 1H), 4.39 (d, J = 15.9 Hz, |
| | 1H), 4.32-4.23 (m, 1H), 4.07 (t, J = 11.4 Hz, 1H), 2.04 (br. s, 6H), 1.76- |
| | 1.69 (overlapped with water, m, 4H), 1.65-1.59 (overlapped with water, m, |
| | 1H), 1.48 (d, J = 7.1 Hz, 3H), 1.45 (d, J = 7.1 Hz, 3H), 0.65 (s, 9H). (1H |
| | missing, labile H). |
| IV-2 | ¹H NMR (400 MHz, CDCl₃) δ 8.90 (t, J = 1.8 Hz, 1H), 8.51 (s, 1H), 7.84- |
| | 7.78 (m, 2H), 7.58 (t, J = 7.8 Hz, 1H), 7.26-7.20 (m, 1H), 7.12 (d, J = 7.6 |
| | Hz, 1H), 7.07 (d, J = 7.6 Hz, 1H), 6.73 (s, 1H), 5.55 (dd, J = 10.5, 3.9 Hz, |
| | 1H), 5.49 (d, J = 16.1 Hz, 1H), 4.87 (d, J = 16.1 Hz, 1H), 4.44 (t, J = 11.1 Hz, |
| | 1H), 4.39-4.30 (m, 1H), 4.08 (s, 3H), 3.72-3.62 (m, 2H), 3.55 (hept, J = 6.0 |
| | Hz, 1H), 2.00 (s, 3H), 1.98 (s, 3H), 1.69 (s, 3H), 1.48 (s, 3H), 1.16 (d, J = 6.1 |
| | Hz, 3H), 1.13 (d, J = 6.1 Hz, 3H), 1.08-0.99 (m, 2H), 0.99-0.90 (m, 2H). |
| IV-3 | ¹H NMR (400 MHz, CDCl₃) δ 8.71 (t, J = 1.8 Hz, 1H), 8.39 (s, 1H), 8.03 (dt, |
| | J = 7.9, 1.5 Hz, 1H), 7.85 (dt, J = 7.6, 1.4 Hz, 1H), 7.63 (t, J = 7.7 Hz, 1H), |
| | 7.23 (t, J = 7.6 Hz, 1H), 7.10 (d, J = 7.6 Hz, 1H), 7.07 (d, J = 7.6 Hz, 1H), |
| | 6.63 (s, 1H), 5.42 (dd, J = 11.3, 4.5 Hz, 1H), 5.36 (d, J = 15.1 Hz, 1H), 4.66- |
| | 4.55 (m, 2H), 4.46-4.36 (m, 1H), 3.74-3.67 (m, 1H), 3.61 (dd, J = 10.6, 3.1 |
| | Hz, 1H), 3.46 (hept, J = 5.9 Hz, 1H), 2.28-2.17 (m, 1H), 2.17-2.07 (m, 1H), |
| | 1.99 (s, 3H), 1.98 (s, 3H), 1.43 (s, 9H), 1.13 (d, J = 6.1 Hz, 3H), 1.07 (d, J = |
| | 6.1 Hz, 3H), 0.86 (t, J = 7.4 Hz, 3H). |
| IV-5 | ¹H NMR (400 MHz, Chloroform-d) δ 8.71 (t, J = 1.8 Hz, 1H), 8.41 (s, 1H), |
| | 8.10 (dt, J = 8.0, 1.5 Hz, 1H), 7.84 (dt, J = 7.7, 1.4 Hz, 1H), 7.65 (t, J = 7.8 |
| | Hz, 1H), 7.23 (d, J = 7.6 Hz, 1H), 7.09 (d, J = 7.6 Hz, 2H), 6.62 (s, 1H), 6.32 |
| | (s, 1H), 5.44 (dd, J = 11.4, 4.3 Hz, 1H), 5.30 (d, J = 14.8 Hz, 1H), 4.43-4.30 |
| | (m, 2H), 4.18 (td, J = 11.0, 4.3 Hz, 1H), 2.06 (s, 6H), 1.99 (dd, J = 15.0, 9.6 |
| | Hz, 2H), 1.88-1.82 (m, 1H), 1.80-1.75 (m, 1H), 1.72-1.66 (m, 2H), 1.57- |
| | 1.51 (m, 1H), 1.43 (s, 9H), 1.39 (d, J = 7.8 Hz, 1H), 0.67 (s, 3H). |
| IV-6 | ¹H NMR (400 MHz, Chloroform-d) δ 8.70 (t, J = 1.8 Hz, 1H), 8.41 (s, 1H), |
| | 8.11 (dt, J = 8.2, 1.4 Hz, 1H), 7.87 (dt, J = 7.9, 1.3 Hz, 1H), 7.66 (t, J = 7.8 |
| | Hz, 1H), 7.23 (d, J = 3.6 Hz, 1H), 7.08 (d, J = 7.6 Hz, 2H), 6.62 (s, 1H), 6.29 |
| | (s, 1H), 5.43 (dd, J = 11.3, 4.2 Hz, 1H), 5.31 (d, J = 14.9 Hz, 1H), 4.40-4.29 |
| | (m, 2H), 4.16 (ddd, J = 11.0, 7.4, 3.6 Hz, 1H), 2.06 (s, 6H), 1.86-1.80 (m, |
| | 2H), 1.43 (s, 9H), 1.39-1.34 (m, 1H), 0.85 (d, J = 6.5 Hz, 3H), 0.41 (d, J = |
| | 6.3 Hz, 3H). |
| IV-7 | ¹H NMR (400 MHz, Chloroform-d) δ 8.65 (t, J = 1.7 Hz, 1H), 8.40 (s, 1H), |
| | 8.08 (d, J = 7.9 Hz, 1H), 7.87-7.82 (m, 1H), 7.65 (t, J = 7.8 Hz, 1H), 7.22 (t, |
| | J = 7.6 Hz, 1H), 7.07 (d, J = 7.6 Hz, 2H), 6.61 (s, 1H), 6.28 (s, 1H), 5.38 (dd, |
| | J = 11.4, 4.2 Hz, 1H), 5.33 (d, J = 14.9 Hz, 1H), 4.34 (t, J = 11.5 Hz, 1H), |
| | 4.24 (d, J = 14.9 Hz, 1H), 4.04 (tt, J = 11.1, 3.9 Hz, 1H), 2.17-2.10 (m, 1H), |
| | 2.06 (s, 6H), 2.03-1.98 (m, 1H), 1.94-1.87 (m, 1H), 1.84-1.76 (m, 1H), |
| | 1.72-1.64 (m, 3H), 1.61-1.56 (m, 1H), 1.43 (s, 9H), 1.08 (p, J = 9.3 Hz, |
| | 1H). |
| IV-8 | ¹H NMR (400 MHz, Chloroform-d) δ 8.68 (t, J = 1.8 Hz, 1H), 8.41 (s, 1H), |
| | 8.12 (dt, J = 8.1, 1.4 Hz, 1H), 7.87 (dt, J = 7.8, 1.4 Hz, 1H), 7.67 (t, J = 7.8 |
| | Hz, 1H), 7.25-7.20 (m, 1H), 7.08 (d, J = 7.7 Hz, 2H), 6.62 (s, 1H), 6.29 (s, |
| | 1H), 5.43 (dd, J = 11.4, 4.2 Hz, 1H), 5.33 (d, J = 14.9 Hz, 1H), 4.37 (t, J = |
| | 11.5 Hz, 1H), 4.31 (d, J = 14.9 Hz, 1H), 4.19-4.07 (m, 1H), 2.07 (s, 6H), |
| | 1.99-1.90 (m, 2H), 1.71-1.64 (m, 2H), 1.55-1.48 (m, 2H), 1.43 (s, 9H), |
| | 1.41-1.36 (m, 2H), 1.28-1.18 (m, 1H), 1.11-1.02 (m, 1H), 0.48-0.38 (m, |
| | 1H). |
| IV-9 | ¹H NMR (400 MHz, Chloroform-d) δ 8.67 (d, J = 1.8 Hz, 1H), 8.42 (s, 1H), |
| | 8.12 (dt, J = 8.0, 1.4 Hz, 1H), 7.82 (dt, J = 7.7, 1.4 Hz, 1H), 7.65 (t, J = 7.8 |
| | Hz, 1H), 7.22 (t, J = 7.6 Hz, 1H), 7.06 (d, J = 7.6 Hz, 2H), 6.61 (s, 1H), 6.24 |
| | (s, 1H), 5.40 (dd, J = 11.1, 4.1 Hz, 1H), 5.30 (d, J = 14.9 Hz, 1H), 4.38-4.27 |
| | (m, 2H), 4.24-4.13 (m, 1H), 2.03 (s, 6H), 1.74 (ddd, J = 14.2, 10.7, 3.0 Hz, |
| | 1H), 1.68-1.58 (m, 2H), 1.55-1.46 (m, 2H), 1.43 (s, 9H), 1.39 (t, J = 2.9 Hz, |
| | 1H), 1.24-1.08 (m, 2H), 1.08-0.99 (m, 2H), 0.97-0.84 (m, 2H), 0.37 (q, J = |
| | 11.9 Hz, 1H). |
| IV-18 | ¹H NMR (400 MHz, CDCl₃) δ 8.71 (s, 1H), 8.48 (s, 1H), 8.05 (d, J = 8.2 Hz, |
| | 1H), 7.84 (d, J = 1.4 Hz, 1H), 7.61 (t, J = 7.8 Hz, 1H), 7.21 (t, J = 7.6 Hz, |
| | 1H), 7.04 (d, J = 7.6 Hz, 2H), 6.93 (s, 1H), 6.21 (s, 1H), 5.42 (dd, J = 11.1, |
| | 4.1 Hz, 1H), 5.29 (d, J = 14.8 Hz, 1H), 4.69 (s, 2H), 4.40 (tdd, J = 9.8, 4.4, |
| | 2.3 Hz, 1H), 4.27 (t, J = 11.5 Hz, 1H), 4.18 (d, J = 14.8 Hz, 1H), 3.81 (hept, J = |
| | 6.1 Hz, 1H), 1.99 (s, 6H), 1.81 (d, 1H), 1.48 (dd, J = 15.2, 9.9 Hz, 1H), 1.26 |
| | (dd, J = 6.1, 1.5 Hz, 6H), 0.50 (s, 3H), 0.38-0.32 (m, 1H), 0.28-0.22 (m, |
| | 1H), 0.17-0.11 (m, 1H), 0.05-0.01 (m, 1H). |
| IV-19 | ¹H NMR (400 MHz, CDCl₃) δ 9.10 (t, J = 1.9 Hz, 1H), 8.48 (s, 1H), 8.10 (dt, |
| | J = 7.9, 1.5 Hz, 1H), 7.89-7.85 (m, 1H), 7.65 (t, J = 7.8 Hz, 1H), 7.26-7.21 |
| | (m, 1H), 7.12 (d, J = 7.7 Hz, 1H), 7.06 (d, J = 7.5 Hz, 1H), 6.44 (s, 1H), 5.65 |
| | (dd, J = 11.0, 4.2 Hz, 1H), 5.43 (d, J = 15.9 Hz, 1H), 4.42 (d, J = 15.9 Hz, |
| | 1H), 4.28-4.20 (m, 1H), 4.09 (t, J = 11.3 Hz, 1H), 4.02 (s, 3H), 2.26-2.12 |
| | (m, 2H), 1.85-1.75 (m, 5H), 1.72 (s, 3H), 1.62 (d, 1H), 1.45 (s, 3H), 1.03- |
| | 0.93 (m, 2H), 0.89-0.84 (m, 2H), 0.81 (d, J = 4.7 Hz, 3H), 0.80 (d, J = 4.5 |
| | Hz, 3H), 0.65 (s, 9H). |
| IV-25 | ¹H NMR (400 MHz, CDCl₃) δ 9.28 (s, 1H), 8.73 (s, 1H), 8.12 (d, J = 1.4 Hz, |
| | 1H), 7.89 (d, J = 1.4 Hz, 1H), 7.66 (t, J = 7.8 Hz, 1H), 7.23 (d, J = 7.7 Hz, |
| | 1H), 7.10 (d, J = 7.7 Hz, 1H), 7.06 (d, J = 7.5 Hz, 1H), 6.14 (s, 1H), 5.70- |
| | 5.59 (m, 2H), 4.61 (d, J = 17.2 Hz, 1H), 4.25-4.18 (m, 1H), 3.98 (s, 3H), 3.83 |
| | (t, J = 11.3 Hz, 1H), 2.22-2.14 (m, 2H), 1.94-1.86 (m, 1H), 1.81 (s, 3H), |
| | 1.80-1.71 (m, 2H), 1.68 (s, 3H), 1.57 (d, J = 1.6 Hz, 1H), 1.06 (dd, J = 8.4, |
| | 2.5 Hz, 2H), 0.83-0.71 (m, 8H), 0.66 (s, 9H). |
| IV-27 | ¹H NMR (400 MHz, CDCl₃) δ 8.74 (s, 1H), 8.43 (s, 1H), 8.07 (d, J = 8.1 Hz, |
| | 1H), 7.86 (d, J = 1.4 Hz, 1H), 7.63 (t, J = 7.8 Hz, 1H), 7.21 (t, J = 7.6 Hz, |
| | 1H), 7.06 (d, J = 7.6 Hz, 2H), 6.67 (s, 1H), 6.24 (s, 1H), 5.42 (dd, J = 11.2, |
| | 4.2 Hz, 1H), 5.28 (d, J = 14.8 Hz, 1H), 4.46-4.37 (m, 1H), 4.28 (t, J = 11.5 |
| | Hz, 1H), 4.16 (d, J = 14.8 Hz, 1H), 4.13-4.06 (m, 2H), 3.58 (td, J = 11.7, 2.2 |
| | Hz, 2H), 3.17-3.08 (m, 1H), 2.62 (s, 1H), 2.11-2.03 (m, 2H), 2.01 (s, 6H), |
| | 1.82 (d, J = 15.0 Hz, 1H), 1.48 (dd, J = 15.1, 10.0 Hz, 2H), 0.49 (s, 3H), 0.38- |
| | 0.32 (m, 1H), 0.28-0.22 (m, 1H), 0.17-0.10 (m, 1H), 0.05-0.01 (m, 1H). |
| IV-33 | ¹H NMR (400 MHz, CDCl₃) δ 8.82 (t, J = 1.8 Hz, 1H), 8.32 (s, 1H), 8.05- |
| | 8.00 (m, 1H), 7.91-7.86 (m, 1H), 7.60 (t, J = 7.8 Hz, 1H), 7.22 (d, J = 7.6 |
| | Hz, 1H), 7.09 (d, J = 7.6 Hz, 2H), 6.64 (s, 1H), 6.39 (s, 1H), 5.59 (dd, J = |
| | 11.7, 5.0 Hz, 1H), 5.38 (d, J = 14.9 Hz, 1H), 4.56 (t, J = 11.9 Hz, 1H), 4.38 |
| | (d, J = 14.9 Hz, 1H), 4.31-4.21 (m, 1H), 2.47-2.33 (m, 1H), 2.31-2.22 (m, |
| | 1H), 2.08 (s, 6H), 2.05-1.96 (m, 2H), 1.94-1.88 (m, 2H), 1.86-1.78 (m, |
| | 2H), 1.73-1.69 (m, 2H), 1.65-1.59 (m, 2H), 1.41 (s, 9H), 0.95-0.82 (m, |
| | 3H). |
| IV-34 | ¹H NMR (400 MHz, CDCl₃) δ 8.76 (t, J = 1.9 Hz, 1H), 8.34 (s, 1H), 8.05- |
| | 7.99 (m, 1H), 7.86-7.80 (m, 1H), 7.61 (t, J = 7.8 Hz, 1H), 7.21 (t, J = 7.6 Hz, |
| | 1H), 7.06 (d, J = 7.6 Hz, 2H), 6.62 (s, 1H), 6.24 (s, 1H), 5.43 (dd, J = 11.0, |
| | 3.8 Hz, 1H), 5.25 (d, J = 14.9 Hz, 1H), 4.39-4.13 (m, 3H), 2.18-2.08 (m, |
| | 1H), 2.00 (s, 6H), 1.76 (dd, J = 15.3, 8.7 Hz, 1H), 1.61 (s, 1H), 1.18-1.11 (m, |
| | 4H), 0.99-0.93 (m, 1H), 0.87-0.82 (m, 1H), 0.61 (s, 3H), 0.59-0.53 (m, |
| | 6H). |
| IV-37 | ¹H NMR (400 MHz, CDCl₃) δ 8.75 (t, J = 1.8 Hz, 1H), 8.41 (s, 1H), 8.05- |
| | 7.99 (m, 1H), 7.83 (dt, J = 7.7, 1.4 Hz, 1H), 7.61 (t, J = 7.8 Hz, 1H), 7.22 (t, J = |
| | 7.6 Hz, 1H), 7.06 (d, J = 7.6 Hz, 2H), 6.62 (s, 1H), 6.25 (s, 1H), 5.45 (dd, J = |
| | 11.3, 4.2 Hz, 1H), 5.27 (d, J = 14.8 Hz, 1H), 4.37-4.26 (m, 2H), 4.22- |
| | 4.13 (m, 1H), 2.00 (s, 6H), 1.76 (dd, J = 15.3, 8.9 Hz, 1H), 1.62 (d, 1H), 1.43 |
| | (s, 9H), 1.01-0.83 (m, 2H), 0.62 (s, 3H), 0.60-0.53 (m, 6H). |
| IV-38 | ¹H NMR (400 MHz, CDCl₃) δ 9.17 (t, J = 1.9 Hz, 1H), 8.90 (s, 1H), 8.07- |
| | 8.01 (m, 1H), 7.89-7.84 (m, 1H), 7.67 (t, J = 7.7 Hz, 1H), 7.23 (t, J = 7.6 Hz, |
| | 1H), 7.08 (d, J = 7.6 Hz, 2H), 6.62 (s, 1H), 6.22 (s, 1H), 5.81 (d, J = 17.4 Hz, |
| | 1H), 5.69-5.61 (m, 1H), 4.85 (d, J = 17.7 Hz, 1H), 4.29-4.20 (m, 1H), 4.16 |
| | (s, 3H), 3.86 (t, J = 11.1 Hz, 1H), 2.03 (s, 6H), 1.83-1.77 (m, 1H), 1.62 (d, J = |
| | 4.0 Hz, 1H), 1.50 (s, 9H), 0.67 (s, 9H). |
| IV-39 | ¹H NMR (400 MHz, DMSO) δ 12.98 (s, 1H), 8.60 (s, 1H), 8.32 (s, 1H), 7.92 |
| | (s, 1H), 7.65 (s, 2H), 7.40-7.30 (m, 2H), 7.25 (s, 1H), 6.91 (s, 1H), 5.35 (s, |
| | 1H), 4.97 (d, J = 15.4 Hz, 1H), 4.68 (d, J = 15.4 Hz, 1H), 4.42 (s, 1H), 4.21 |
| | (s, 2H), 4.04 (s, 1H), 3.43 (s, 1H), 2.26 (ddd, J = 13.2, 8.5, 5.1 Hz, 1H), 1.90 |
| | (dd, J = 14.8, 9.1 Hz, 1H), 1.81 (s, 3H), 1.62 (s, 3H), 1.42 (d, J = 15.2 Hz, |
| | 1H), 1.17-1.05 (m, 4H), 1.01 (d, J = 6.1 Hz, 6H), 0.54 (s, 9H). |
| IV-41 | ¹H NMR (400 MHz, CDCl₃) δ 8.84 (t, J = 1.8 Hz, 1H), 8.58 (s, 1H), 8.09 (dt, |
| | J = 8.1, 1.3 Hz, 1H), 7.87 (dt, J = 7.6, 1.4 Hz, 1H), 7.65 (t, J = 7.8 Hz, 1H), |
| | 7.21 (t, J = 7.6 Hz, 1H), 7.16 (d, J = 0.8 Hz, 1H), 7.07 (d, J = 7.6 Hz, 2H), |
| | 6.26 (s, 1H), 5.45 (dd, J = 11.4, 4.3 Hz, 1H), 5.31 (d, J = 15.1 Hz, 1H), 4.48- |
| | 4.37 (m, 1H), 4.25-4.15 (m, 2H), 2.25 (ddd, J = 13.3, 8.5, 4.9 Hz, 1H), 2.03 |
| | (s, 6H), 1.82 (d, J = 14.9 Hz, 1H), 1.47 (dd, J = 15.2, 10.1 Hz, 1H), 1.23- |
| | 1.15 (m, 2H), 1.02-0.93 (m, 2H), 0.49 (s, 3H), 0.42-0.33 (m, 1H), 0.29- |
| | 0.21 (m, 1H), 0.17-0.11 (m, 1H), 0.05-0.01 (m, 1H). |
| IV-43 | ¹H NMR (400 MHz, CDCl₃) δ 8.71 (s, 1H), 8.39 (s, 1H), 8.05 (d, 1H), 7.85 (d, |
| | J = 1.7 Hz, 1H), 7.62 (t, J = 2.0 Hz, 1H), 7.25 (t, 1H), 7.11 (d, J = 7.7 Hz, |
| | 1H), 7.07 (d, J = 7.5 Hz, 1H), 6.63 (s, 1H), 5.45-5.40 (m, 1H), 5.38 (d, 1H), |
| | 4.64-4.55 (m, 2H), 4.43-4.34 (m, 1H), 3.72-3.65 (m, 1H), 3.58 (dd, J = |
| | 10.7, 3.2 Hz, 1H), 3.46 (hept, J = 6.1 Hz, 1H), 2.23-2.08 (m, 2H), 1.92 (s, |
| | 3H), 1.83-1.72 (m, 1H), 1.71 (s, 3H), 1.43 (s, 9H), 1.12 (d, J = 6.0 Hz, 3H), |
| | 1.06 (d, J = 6.1 Hz, 3H), 0.80 (dd, J = 6.6, 1.4 Hz, 6H). |
| IV-44 | ¹H NMR (400 MHz, CDCl₃) δ 9.04 (s, 1H), 8.48 (s, 1H), 8.09 (d, J = 1.6 Hz, |
| | 1H), 7.89 (d, J = 1.4 Hz, 1H), 7.65 (t, J = 7.8 Hz, 1H), 7.24 (t, J = 7.6 Hz, |
| | 1H), 7.12 (d, J = 7.7 Hz, 1H), 7.07 (d, J = 7.5 Hz, 1H), 6.54 (s, 1H), 5.59 (dd, |
| | J = 11.1, 4.4 Hz, 1H), 5.43 (d, J = 15.7 Hz, 1H), 4.50-4.41 (m, 1H), 4.26 (d, |
| | J = 15.7 Hz, 1H), 4.17-4.08 (m, 4H), 2.25-2.11 (m, 2H), 1.86-1.75 (m, |
| | 5H), 1.72 (s, 3H), 1.51 (s, 8H), 1.48-1.40 (m, 2H), 0.80 (dd, J = 6.6, 5.2 Hz, |
| | 6H), 0.47 (s, 3H), 0.44-0.36 (m, 1H), 0.31-0.25 (m, 1H), 0.18-0.12 (m, |
| | 1H), 0.05-0.01 (m, 1H). |
| IV-45 | ¹H NMR (400 MHz, CDCl₃) δ 8.74 (s, 1H), 8.42 (s, 1H), 8.06 (d, 1H), 7.87 (d, |
| | 1H), 7.62 (t, J = 7.8 Hz, 1H), 7.26 (t, J = 4.6 Hz, 1H), 7.12 (d, J = 7.7 Hz, |
| | 1H), 7.06 (d, J = 7.5 Hz, 1H), 6.63 (s, 1H), 5.43 (dd, J = 10.2, 3.3 Hz, 1H), |
| | 5.29 (d, J = 14.7 Hz, 1H), 4.50-4.36 (m, 2H), 4.17 (d, J = 14.7 Hz, 1H), 2.26- |
| | 2.11 (m, 2H), 1.86-1.71 (m, 8H), 1.52-1.46 (m, 1H), 1.43 (s, 9H), 0.81 (dd, |
| | J = 6.6, 3.2 Hz, 6H), 0.42 (s, 3H), 0.40-0.32 (m, 1H), 0.27-0.21 (m, 1H), |
| | 0.16-0.09 (m, 1H), −0.01-−0.05 (m, 1H). |
| IV-47 | ¹H NMR (400 MHz, CDCl₃) δ 9.22 (t, J = 1.8 Hz, 1H), 8.85 (s, 1H), 8.13 (dt, |
| | J = 8.0, 1.4 Hz, 1H), 7.88 (dt, J = 7.8, 1.3 Hz, 1H), 7.68 (t, J = 7.8 Hz, 1H), |
| | 7.26-7.24 (m, 1H), 7.12 (d, J = 7.7 Hz, 1H), 7.07 (d, J = 7.5 Hz, 1H), 6.53 (s, |
| | 1H), 5.77 (d, J = 17.4 Hz, 1H), 5.68 (dd, J = 10.8, 4.1 Hz, 1H), 4.82 (d, J = |
| | 17.6 Hz, 1H), 4.30-4.21 (m, 1H), 4.15 (s, 3H), 3.91 (t, J = 11.2 Hz, 1H), 2.25- |
| | 2.12 (m, 2H), 1.84-1.75 (m, 5H), 1.70 (s, 3H), 1.58 (s, 1H), 1.49 (s, 9H), |
| | 0.83-0.77 (m, 6H), 0.66 (s, 9H). |
| IV-50 | ¹H NMR (400 MHz, CDCl₃) δ 9.01 (s, 1H), 8.47 (s, 1H), 7.91 (t, J = 7.1 Hz, |
| | 2H), 7.59 (t, J = 7.7 Hz, 1H), 7.23 (d, J = 7.6 Hz, 1H), 7.10 (s, 1H), 7.08 (s, |
| | 1H), 6.54 (s, 1H), 6.39 (s, 1H), 5.67 (d, J = 8.2 Hz, 1H), 5.49 (d, J = 15.0 Hz, |
| | 1H), 4.48 (d, J = 15.2 Hz, 1H), 4.43-4.30 (m, 2H), 4.09 (s, 3H), 2.48-2.36 |
| | (m, 1H), 2.33-2.20 (m, 1H), 2.08 (s, 6H), 2.06-1.78 (m, 7H), 1.77-1.67 (m, |
| | 2H), 1.67-1.58 (m, 2H), 1.49 (s, 9H), 1.28-1.16 (m, 1H), 0.94-0.81 (m, |
| | 1H). |
| IV-62 | ¹H NMR (400 MHz, CDCl₃) δ 8.79 (s, 1H), 8.21 (s, 1H), 8.05 (d, J = 7.8 Hz, |
| | 1H), 7.83 (d, J = 7.7 Hz, 1H), 7.63 (t, J = 7.8 Hz, 1H), 7.38-7.27 (m, 4H), |
| | 7.24-7.19 (m, 1H), 7.07 (d, J = 7.6 Hz, 2H), 6.64 (s, 1H), 6.13 (s, 1H), 5.43- |
| | 5.35 (m, 2H), 5.01 (d, J = 15.5 Hz, 1H), 4.78 (t, J = 11.5 Hz, 1H), 4.70 (d, J = |
| | 11.7 Hz, 1H), 4.45 (d, J = 11.7 Hz, 1H), 4.10 (dt, J = 11.5, 4.3 Hz, 1H), 3.67 |
| | (dd, J = 6.4, 3.9 Hz, 1H), 2.11 (ddd, J = 13.6, 8.2, 5.3 Hz, 1H), 2.04 (s, 6H), |
| | 1.14 (d, J = 6.4 Hz, 5H), 1.12-1.08 (m, 1H), 0.98-0.81 (m, 2H). |
| IV-65 | ¹H NMR (400 MHz, CDCl₃) δ 8.75 (t, J = 2.2 Hz, 1H), 8.41 (s, 1H), 8.08 |
| | (d, J = 7.9 Hz, 1H), 7.87 (d, J = 7.6 Hz, 1H), 7.64 (t, J = 7.8 Hz, 1H), 7.21 (t, J = |
| | 7.6 Hz, 1H), 7.08 (s, 1H), 7.06 (s, 1H), 6.71 (s, 1H), 6.26 (s, 1H), 5.41 (dd, J = |
| | 11.3, 4.2 Hz, 1H), 5.29 (d, J = 14.8 Hz, 1H), 4.48-4.37 (m, 1H), 4.29 (t, J = |
| | 11.5 Hz, 1H), 4.17 (d, J = 14.8 Hz, 1H), 3.89 (p, J = 8.1 Hz, 1H), 2.64-2.56 |
| | (m, 2H), 2.55-2.45 (m, 2H), 2.02 (s, 6H), 1.54-1.48 (m, 2H), 0.59-0.50 (m, |
| | 4H), 0.48 (s, 3H), 0.40-0.31 (m, 1H), 0.28-0.21 (m, 1H), 0.18-0.09 (m, |
| | 1H), 0.05-0.01 (m, OH), 0.01 (s, OH), 0.06-−0.00 (m, 1H). |
| IV-66 | ¹H NMR (400 MHz, CDCl₃) δ 9.05 (s, 1H), 8.49 (s, 1H), 8.09-8.04 (m, 1H), |
| | 7.89 (d, J = 1.4 Hz, 1H), 7.66 (t, J = 7.8 Hz, 1H), 7.32 (t, J = 7.6 Hz, 1H), |
| | 7.13 (s, 2H), 6.52 (s, 1H), 6.30 (s, 1H), 5.62 (dd, J = 11.2, 4.2 Hz, 1H), 5.40 |
| | (d, J = 15.8 Hz, 1H), 4.40 (d, J = 15.8 Hz, 1H), 4.32-4.23 (m, 1H), 4.10 (s, |
| | 3H), 2.40-2.11 (m, 4H), 1.77 (dd, J = 15.1, 8.8 Hz, 1H), 1.67-1.56 (m, 2H), |
| | 1.50 (s, 9H), 1.10 (s, 3H), 0.96 (s, 3H), 0.67 (s, 9H). |
| IV-69 | ¹H NMR (400 MHz, CDCl₃) δ 8.87 (s, 1H), 8.51 (s, 1H), 8.11 (d, J = 7.8 Hz, |
| | 1H), 7.88 (d, J = 7.6 Hz, 1H), 7.70 (t, J = 7.7 Hz, 1H), 7.29 (d, J = 7.6 Hz, |
| | 1H), 7.11 (dd, J = 15.6, 7.6 Hz, 2H), 6.57 (s, 1H), 5.67-5.59 (m, 1H), 5.49 (d, |
| | J = 15.3 Hz, 1H), 4.64 (d, J = 16.8 Hz, 2H), 4.45 (t, J = 11.5 Hz, 1H), 4.33 (t, |
| | J = 9.5 Hz, 1H), 4.16 (d, J = 10.4 Hz, 1H), 4.09 (s, 3H), 2.21-2.13 (m, 2H), |
| | 2.10-2.04 (m, 1H), 1.92 (s, 3H), 1.82-1.77 (m, 1H), 1.72 (s, 3H), 1.32 (d, J = |
| | 8.4 Hz, 2H), 1.06-0.99 (m, 2H), 0.81 (dd, J = 6.6, 1.9 Hz, 6H). |
| IV-72 | ¹H NMR (400 MHz, CDCl₃) δ 8.58 (s, 1H), 8.22 (d, J = 7.8 Hz, 1H), 8.02 |
| | (t, J = 7.8 Hz, 1H), 7.94 (d, J = 7.7 Hz, 1H), 7.29 (t, J = 7.6 Hz, 1H), 7.14 (d, J = |
| | 7.7 Hz, 1H), 7.09 (d, J = 7.6 Hz, 1H), 6.72 (s, 1H), 6.08 (dd, J = 10.2, 4.9 Hz, |
| | 1H), 5.36 (d, J = 15.4 Hz, 1H), 4.66-4.62 (m, 1H), 4.45 (d, J = 15.5 Hz, 1H), |
| | 4.24-4.18 (m, 1H), 4.17 (s, 3H), 2.29-2.16 (m, 2H), 1.88 (s, 3H), 1.87-1.76 |
| | (m, 2H), 1.73 (s, 3H), 1.54-1.48 (m, 1H), 1.51 (s, 9H), 0.84 (d, J = 6.8 Hz, |
| | 3H), 0.81 (d, J = 6.9 Hz, 3H), 0.49 (s, 3H), 0.42-0.34 (m, 1H), 0.28-0.20 |
| | (m, 1H), 0.17-0.08 (m, 1H), 0.06-0.00 (m, 1H). |
| IV-73 | ¹H NMR (400 MHz, CDCl₃) δ 9.00 (t, J = 1.9 Hz, 1H), 8.59 (s, 1H), 8.11 |
| | (d, J = 7.9 Hz, 1H), 7.92 (dt, J = 7.7, 1.3 Hz, 1H), 7.69 (t, J = 7.8 Hz, 1H), 7.25- |
| | 7.20 (m, 1H), 7.08 (d, J = 7.6 Hz, 2H), 6.84 (s, 1H), 6.26 (s, 1H), 5.64 (dd, J = |
| | 10.5, 3.2 Hz, 1H), 5.41 (d, J = 15.9 Hz, 1H), 4.34 (d, J = 16.0 Hz, 1H), 4.15- |
| | 4.08 (m, 1H), 4.05 (d, J = 10.8 Hz, 1H), 4.02 (s, 3H), 2.47 (s, 1H), 2.08 (s, |
| | 6H), 1.96 (dd, J = 15.5, 10.1 Hz, 1H), 1.87-1.82 (m, 1H), 1.62-1.56 (m, |
| | 8H), 1.24 (d, J = 13.5 Hz, 2H). |
| IV-74 | ¹H NMR (400 MHz, CDCl₃) δ 8.92 (t, J = 1.8 Hz, 1H), 8.57 (s, 1H), 7.85 (dt, |
| | J = 7.7, 1.3 Hz, 1H), 7.77 (d, J = 7.9 Hz, 1H), 7.60 (t, J = 7.8 Hz, 1H), 7.23 |
| | (d, J = 7.6 Hz, 1H), 7.09 (d, J = 7.6 Hz, 2H), 6.80 (s, 1H), 6.25 (s, 1H), 5.55 |
| | (d, J = 7.0 Hz, 1H), 5.47 (d, J = 16.0 Hz, 1H), 4.71 (d, J = 16.0 Hz, 1H), 4.40- |
| | 4.30 (m, 2H), 4.01 (s, 3H), 3.69-3.60 (m, 2H), 3.53 (hept, J = 6.1 Hz, 1H), |
| | 2.08 (s, 6H), 1.61-1.57 (m, 2H), 1.24 (q, J = 9.9 Hz, 2H), 1.13 (dd, J = 16.2, |
| | 6.1 Hz, 6H). |
| IV-75 | ¹H NMR (400 MHz, CDCl₃) δ 9.00 (s, 1H), 8.60 (s, 1H), 8.09 (d, J = 7.9 Hz, |
| | 1H), 7.89 (d, J = 7.6 Hz, 1H), 7.66 (t, J = 7.8 Hz, 1H), 7.23 (t, J = 7.6 Hz, |
| | 1H), 7.09 (d, J = 7.6 Hz, 2H), 6.86 (s, 1H), 6.28 (s, 1H), 5.62 (dd, J = 11.2, |
| | 4.2 Hz, 1H), 5.40 (d, J = 15.8 Hz, 1H), 4.51-4.40 (m, 1H), 4.28 (d, J = 15.8 |
| | Hz, 1H), 4.10 (t, J = 11.5 Hz, 1H), 4.04 (s, 3H), 2.05 (s, 6H), 1.86 (d, J = 15.0 |
| | Hz, 1H), 1.64-1.58 (m, 2H), 1.45 (dd, J = 15.1, 10.2 Hz, 1H), 1.25 (q, J = 9.9 |
| | Hz, 2H), 0.53 (s, 3H), 0.42-0.36 (m, 1H), 0.32-0.25 (m, 1H), 0.20-0.14 (m, |
| | 1H), 0.08-0.02 (m, 1H). |
| IV-77 | ¹H NMR (400 MHz, CDCl₃) δ 9.04 (t, J = 1.8 Hz, 1H), 8.58 (s, 1H), 8.10 (dt, |
| | J = 8.1, 1.3 Hz, 1H), 7.88 (dt, J = 7.8, 1.3 Hz, 1H), 7.65 (t, J = 7.8 Hz, 1H), |
| | 7.25-7.21 (m, 1H), 7.12 (d, J = 7.6 Hz, 1H), 7.07 (d, J = 7.5 Hz, 1H), 6.78 (s, |
| | 1H), 5.63 (dd, J = 11.2, 4.4 Hz, 1H), 5.42 (d, J = 15.8 Hz, 1H), 4.50-4.41 (m, |
| | 1H), 4.27 (d, J = 15.9 Hz, 1H), 4.13 (t, J = 11.5 Hz, 1H), 4.03 (s, 3H), 2.25- |
| | 2.13 (m, 2H), 1.91-1.84 (m, 1H), 1.83 (s, 3H), 1.80-1.75 (m, 1H), 1.73 (s, |
| | 3H), 1.59 (d, J = 109.2 Hz, 2H), 1.44 (dd, J = 14.3, 10.7 Hz, 1H), 1.24 (q, J = |
| | 10.2 Hz, 2H), 0.81 (dd, J = 6.6, 5.4 Hz, 6H), 0.48 (s, 3H), 0.43-0.37 (m, 1H), |
| | 0.31-0.25 (m, 1H), 0.18-0.13 (m, 1H), 0.06-0.01 (m, 1H). |
| IV-78 | ¹H NMR (400 MHz, MeOD) δ 8.49 (s, 1H), 8.21-8.10 (m, 2H), 7.85 (dd, J = |
| | 7.1, 1.5 Hz, 1H), 7.36 (t, J = 7.6 Hz, 1H), 7.22 (d, J = 7.7 Hz, 1H), 7.19 (d, J = |
| | 7.6 Hz, 1H), 6.76 (s, 1H), 5.84-5.77 (m, 1H), 5.10 (d, J = 15.1 Hz, 1H), |
| | 4.59 (d, J = 15.1 Hz, 1H), 4.54-4.43 (m, 2H), 2.30 (d, J = 7.3 Hz, 2H), 1.94 |
| | (s, 3H), 1.86-1.73 (m, 3H), 1.73 (s, 3H), 1.44 (s, 9H), 0.86 (d, J = 6.5 Hz, |
| | 3H), 0.85 (d, J = 6.4 Hz, 3H), 0.44 (s, 3H), 0.43-0.35 (m, 1H), 0.24-0.17 |
| | (m, 1H), 0.16-0.08 (m, 1H), 0.07-0.00 (m, 1H). |
| IV-80 | ¹H NMR (400 MHz, CDCl₃) δ 8.77 (s, 1H), 8.30 (s, 1H), 8.03 (d, J = 8.0 Hz, |
| | 1H), 7.88 (d, J = 7.7 Hz, 1H), 7.63 (t, J = 7.8 Hz, 1H), 7.22 (t, J = 9.0 Hz, |
| | 1H), 7.08 (d, J = 7.7 Hz, 2H), 6.65 (s, 1H), 6.31 (s, 1H), 5.54 (dd, J = 11.8, |
| | 4.3 Hz, 1H), 5.35 (d, J = 15.0 Hz, 1H), 4.54 (t, J = 11.9 Hz, 1H), 4.42-4.31 |
| | (m, 2H), 3.92-3.85 (m, 1H), 2.16-2.09 (m, 1H), 2.04 (s, 6H), 1.21 (d, J = |
| | 6.4 Hz, 3H), 1.18-1.10 (m, 4H), 0.90 (s, 9H). |
| IV-83 | ¹H NMR (400 MHz, CDCl₃) δ 8.63 (t, J = 1.8 Hz, 1H), 8.41 (s, 1H), 7.94 |
| | (d, J = 7.9 Hz, 1H), 7.83 (dt, J = 7.7, 1.3 Hz, 1H), 7.61 (t, J = 7.8 Hz, 1H), 7.23 (t, |
| | J = 7.6 Hz, 1H), 7.08 (d, J = 7.6 Hz, 2H), 6.62 (s, 1H), 6.24 (s, 1H), 5.44 (d, |
| | J = 7.1 Hz, 1H), 5.39 (d, J = 14.8 Hz, 1H), 4.41-4.28 (m, 2H), 4.23 (d, J = |
| | 14.8 Hz, 1H), 3.68-3.51 (m, 3H), 2.08 (s, 6H), 2.04-1.92 (m, 2H), 1.86- |
| | 1.72 (m, 3H), 1.43 (s, 9H), 1.40-1.32 (m, 1H). |
| IV-84 | ¹H NMR (400 MHz, CDCl₃) δ 8.62 (t, J = 1.8 Hz, 1H), 8.34 (s, 1H), 7.86- |
| | 7.77 (m, 2H), 7.57 (t, J = 7.8 Hz, 1H), 7.23 (t, J = 7.6 Hz, 1H), 7.07 (d, J = |
| | 7.6 Hz, 2H), 6.65 (s, 1H), 6.21 (s, 1H), 5.48-5.32 (m, 2H), 4.33-4.15 (m, |
| | 3H), 3.70-3.50 (m, 3H), 2.06 (s, 6H), 2.04-1.90 (m, 2H), 1.86-1.72 (m, |
| | 3H), 1.54 (s, 3H), 1.45-1.30 (m, 3H), 0.99-0.90 (m, 2H). |
| IV-86 | ¹H NMR (400 MHz, CDCl₃) δ 9.02 (s, 1H), 8.66 (s, 1H), 8.34 (s, 1H), 7.79 (d, |
| | J = 1.3 Hz, 1H), 7.72 (d, J = 7.9 Hz, 1H), 7.54 (t, J = 7.8 Hz, 1H), 7.23 (t, J = |
| | 7.6 Hz, 1H), 7.09 (s, 1H), 7.07 (s, 1H), 6.62 (s, 1H), 6.21 (s, 1H), 5.48-5.36 |
| | (m, 2H), 4.45-4.34 (m, 1H), 4.29-4.16 (m, 2H), 3.78 (d, J = 11.3 Hz, 1H), |
| | 3.39 (t, J = 11.0 Hz, 1H), 3.20 (t, J = 10.7 Hz, 1H), 2.09 (s, 6H), 1.92-1.72 |
| | (m, 3H), 1.55 (s, 6H), 1.20-1.11 (m, 4H). |
| IV-95 | ¹H NMR (400 MHz, CDCl₃) δ 8.98 (s, 1H), 8.47 (s, 1H), 8.10 (d, J = 7.7 Hz, |
| | 1H), 7.88 (d, J = 7.3 Hz, 1H), 7.67 (t, J = 7.6 Hz, 1H), 7.29 (d, J = 7.8 Hz, |
| | 1H), 7.11 (dd, J = 19.5, 7.7 Hz, 2H), 6.86 (s, 1H), 5.61 (s, 1H), 5.47 (d, J = |
| | 14.4 Hz, 1H), 5.12-5.01 (m, 1H), 4.66 (s, 1H), 4.32-4.22 (m, 1H), 4.19 (s, |
| | 3H), 4.08-4.00 (m, 1H), 3.99-3.91 (m, 1H), 3.87-3.77 (m, 1H), 2.98-2.86 |
| | (m, 1H), 2.25-2.12 (m, 2H), 2.02-1.95 (m, 2H), 1.94 (s, 3H), 1.90-1.85 (m, |
| | 1H), 1.83-1.78 (m, 1H), 1.75 (s, 3H), 1.53 (s, 9H), 0.85-0.77 (m, 6H). |
| IV-99 | ¹H NMR (400 MHz, CDCl₃) δ 8.89 (t, J = 1.9 Hz, 1H), 8.58 (s, 1H), 7.96 |
| | (d, J = 8.0 Hz, 1H), 7.88 (d, J = 7.7 Hz, 1H), 7.65 (t, J = 7.7 Hz, 1H), 7.23 (d, J = |
| | 7.6 Hz, 1H), 7.09 (d, J = 7.6 Hz, 2H), 6.94 (s, 1H), 6.28 (s, 1H), 5.54 (d, J = |
| | 6.8 Hz, 1H), 5.45 (d, J = 16.0 Hz, 1H), 4.79 (d, J = 16.0 Hz, 1H), 4.45-4.36 |
| | (m, 2H), 4.17 (s, 3H), 3.69-3.59 (m, 2H), 3.53 (p, J = 6.1 Hz, 1H), 2.09 (s, |
| | 6H), 1.78 (s, 6H), 1.15 (d, J = 6.1 Hz, 3H), 1.11 (d, J = 6.1 Hz, 3H). |
| IV-100 | ¹H NMR (400 MHz, CDCl₃) δ 8.95 (t, J = 1.7 Hz, 1H), 8.57 (s, 1H), 8.03 (dt, |
| | J = 8.1, 1.4 Hz, 1H), 7.87 (dt, J = 7.7, 1.3 Hz, 1H), 7.64 (t, J = 7.8 Hz, 1H), |
| | 7.23 (t, J = 7.6 Hz, 1H), 7.09 (dd, J = 13.0, 7.5 Hz, 2H), 6.81 (s, 1H), 5.56 (d, |
| | J = 6.9 Hz, 1H), 5.50 (d, J = 16.0 Hz, 1H), 4.75 (d, J = 16.0 Hz, 1H), 4.48- |
| | 4.37 (m, 2H), 4.14 (s, 3H), 3.71-3.60 (m, 2H), 3.52 (h, J = 6.1 Hz, 1H), 1.99 |
| | (d, J = 17.0 Hz, 6H), 1.77 (s, 6H), 1.72 (s, 3H), 1.16 (d, J = 6.1 Hz, 3H), 1.12 |
| | (d, J = 6.1 Hz, 3H). |
| IV-101 | ¹H NMR (400 MHz, CDCl₃) δ 8.97 (t, J = 1.8 Hz, 1H), 8.56 (s, 1H), 8.07 |
| | (ddd, J = 7.9, 1.9, 1.1 Hz, 1H), 7.88 (dt, J = 7.8, 1.3 Hz, 1H), 7.65 (t, J = 7.8 |
| | Hz, 1H), 7.24 (d, J = 7.6 Hz, 1H), 7.09 (dd, J = 17.4, 7.6 Hz, 2H), 6.78 (s, |
| | 1H), 5.59-5.47 (m, 2H), 4.73 (d, J = 16.0 Hz, 1H), 4.46-4.36 (m, 2H), 4.13 |
| | (s, 3H), 3.65 (qd, J = 10.7, 4.3 Hz, 2H), 3.52 (hept, J = 6.0 Hz, 1H), 2.24- |
| | 2.08 (m, 2H), 1.93 (s, 3H), 1.77 (s, 7H), 1.70 (s, 3H), 1.15 (d, J = 6.1 Hz, 3H), |
| | 1.11 (d, J = 6.1 Hz, 3H), 0.80 (dd, J = 6.6, 3.1 Hz, 6H). |
| IV-103 | ¹H NMR (400 MHz, CDCl₃) δ 8.93 (t, J = 1.8 Hz, 1H), 8.58 (s, 1H), 7.99 |
| | (d, J = 7.9 Hz, 1H), 7.85 (dt, J = 7.7, 1.4 Hz, 1H), 7.64 (t, J = 7.8 Hz, 1H), 7.23 (t, |
| | J = 7.5 Hz, 1H), 7.09 (d, J = 7.6 Hz, 2H), 6.84 (s, 1H), 6.25 (s, 1H), 5.64 (dd, |
| | J = 11.2, 4.0 Hz, 1H), 5.50 (d, J = 15.8 Hz, 1H), 4.44-4.37 (m, 1H), 4.34 (d, |
| | J = 15.9 Hz, 1H), 4.10 (t, J = 11.4 Hz, 1H), 4.04 (s, 3H), 3.75-3.66 (m, 1H), |
| | 3.66-3.53 (m, 2H), 2.09 (s, 6H), 2.06-1.96 (m, 2H), 1.88-1.77 (m, 3H), |
| | 1.62-1.59 (m, 2H), 1.43-1.38 (m, 1H), 1.30-1.20 (m, 2H). |

### C. Bioactivity Data

### 1. HBE Assay Procedure

### (a) Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂, 10 Glucose, 10 HEPES (pH adjusted to 7.4 with NaOH) and the apical solution contained (in mM) 145 NaGluconate, 1.2 MgCl₂, 1.2 CaCl₂, 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

### (b) Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 33 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and 18 μM Tezacaftor or were incubated with the corrector compounds at a single fixed concentration of 1 μM in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and 18 μM Tezacaftor for 18-24 hours at 37° C. and in the presence of 20% human serum. The concentration of corrector compounds with 44 nM or 33 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and 18 μM Tezacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

### (c) Ranges of Concentrations to Measure Potency and Efficacy

Assay A: 0.1 μM to 25 μM, 6 concentrations
Assay B: 0.00001 μM to 25 μM, 12 concentrations
Assay C: 0.026 μM to 25 μM, 6 concentrations
Assay D: Activity at single concentration of 1 μM

### 2. Biological Activity Data

The following tables represents CFTR modulating activity for representative compounds of the invention generated using one or more of the assays disclosed herein.

% Activity: +++ is >60%; ++ is 30-60%; + is detectable to <30%; EC50: +++ is <1 μM; ++ is 1 to <3 μM; and + is 3 to <30 μM.

| | | A Max | B Max | C Max | D activity |
|---|---|---|---|---|---|
| Compound | | activity | activity | activity | at 1 μM |
| Number | Structure | (%) | (%) | (%) | (%) |
| I-1 | 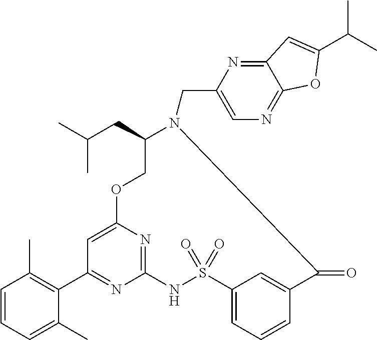 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)C)CO2] | +++ | | | |
| I-2 | 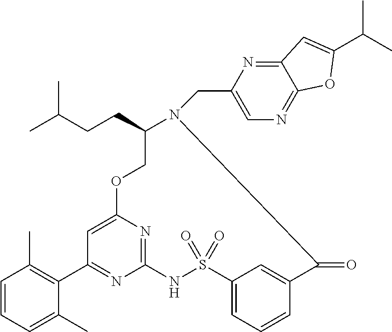 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)C)CO2] | +++ | | | |
| I-3 | 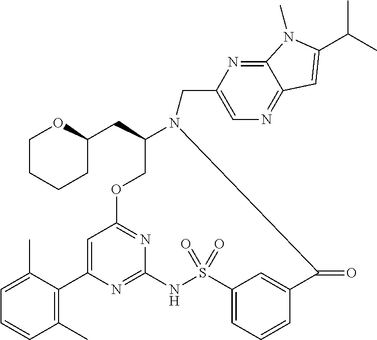 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCCO1)CO2] | +++ | | | |
| I-4 | 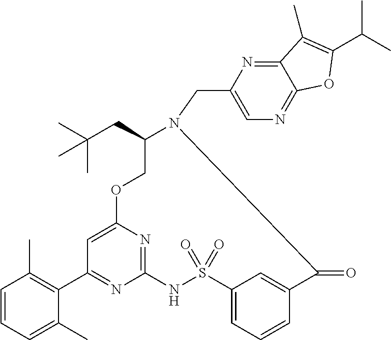 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)C)CO2] | +++ | | | |
| I-5 | 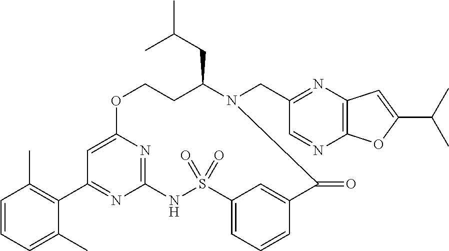 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)C)CCO2] | | | | +++ |
| I-6 | 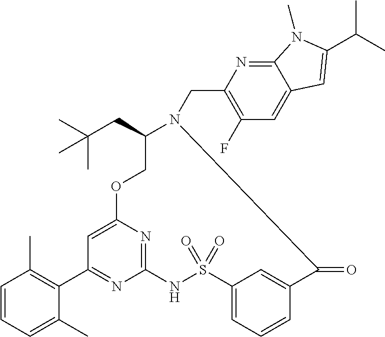 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC(C)(C)C)CO2] | +++ | | | |
| I-7 | 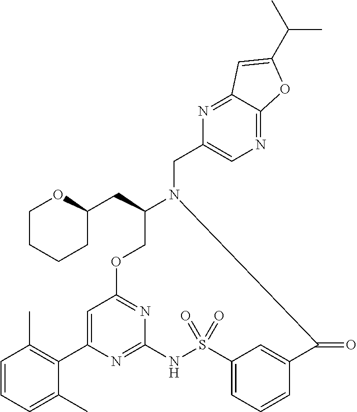 [SMILES: Cc1cccc(C)c1-c1cc2nc(n1)NS(=O)(=O)c1cccc(c1)C(=O)N(Cc1cnc3oc(C(C)C)cc3n1)[C@H](C[C@H]1CCCCO1)CO2] | +++ | | | |
| I-8 | 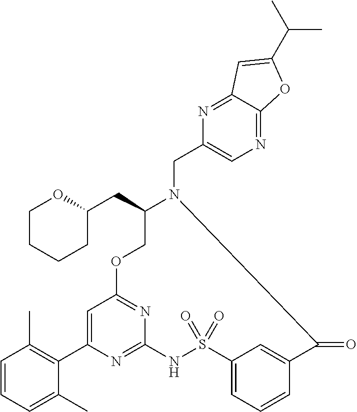 [SMILES: Cc1cccc(C)c1-c1cc2nc(n1)NS(=O)(=O)c1cccc(c1)C(=O)N(Cc1cnc3oc(C(C)C)cc3n1)[C@H](C[C@@H]1CCCCO1)CO2] | | | | +++ |
| I-9 | 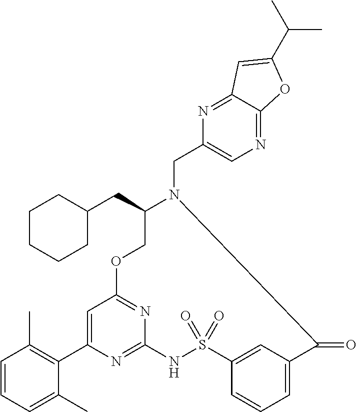 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCCC1)CO2] | +++ | | | |
| I-10 | 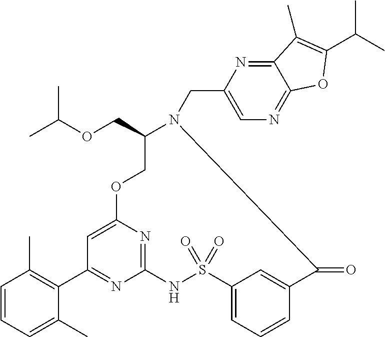 [SMILES: Cc1cccc(C)c1-c1cc2nc(n1)NS(=O)(=O)c1cccc(c1)C(=O)N(Cc1cnc3oc(C(C)C)c(C)c3n1)[C@H](COC(C)C)CO2] | +++ | | | |
| I-11 | 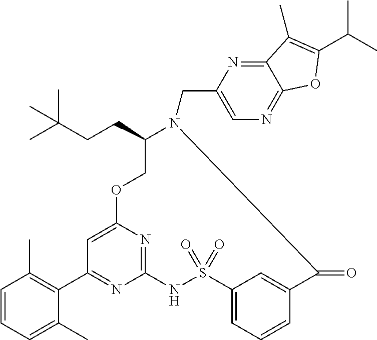 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)(C)C)CO2] | +++ | | | |
| I-12 | 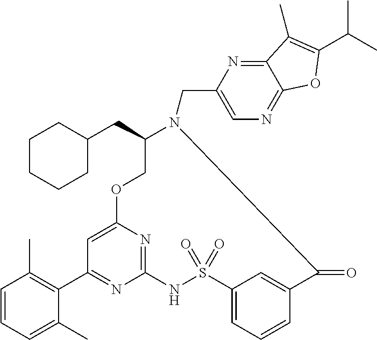 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCCC1)CO2] | +++ | | | |
| I-13 | 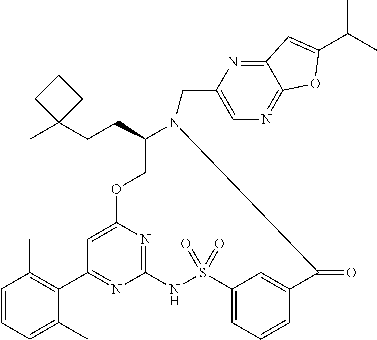 [SMILES: Cc1cccc(C)c1-c1cc2nc(n1)NS(=O)(=O)c1cccc(c1)C(=O)N(Cc1cnc3oc(C(C)C)cc3n1)[C@H](CCC1(C)CCC1)CO2] | +++ | | | |
| I-15 | 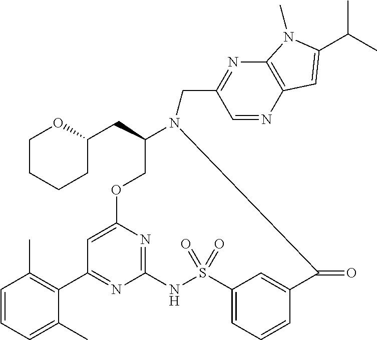 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCCO1)CO2] | | | | +++ |
| I-16 | 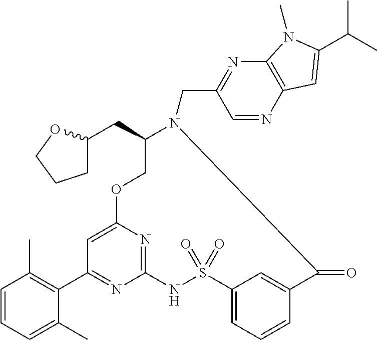 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCO1)CO2] | +++ | | | |
| I-17 | 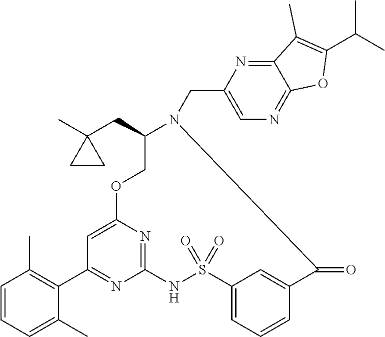 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1(C)CC1)CO2] | +++ | | | |
| I-18 | 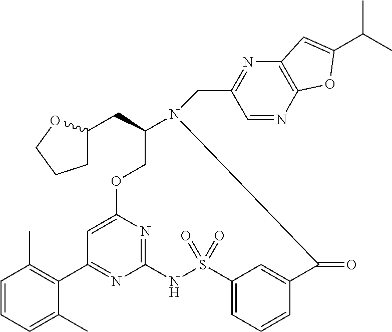 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCO1)CO2] | +++ | | | |
| I-19 | 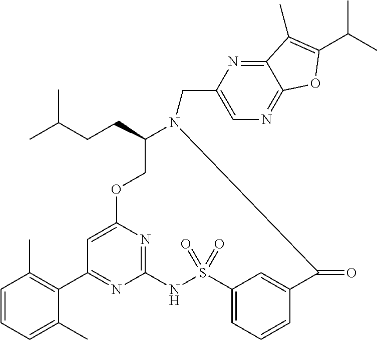 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)C)CO2] | +++ | | | |
| I-20 | 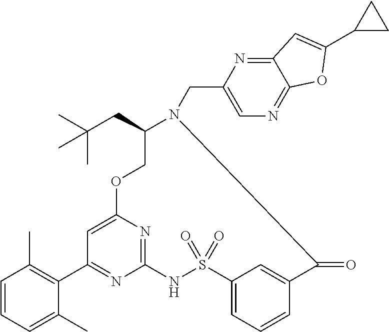 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)C)CO2] | +++ | | | |
| I-21 | 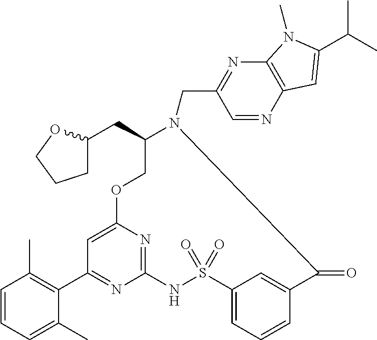 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCO1)CO2] | +++ | | | |
| I-22 | 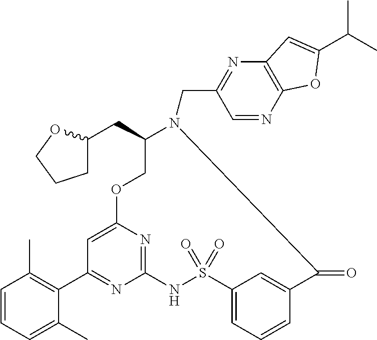 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCO1)CO2] | | | | +++ |
| I-23 | 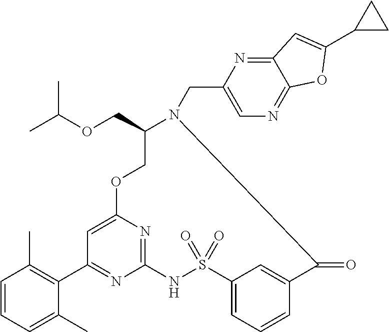 [SMILES: Cc1cccc(C)c1-c1cc2nc(n1)NS(=O)(=O)c1cccc(c1)C(=O)N(Cc1cnc3oc(C4CC4)cc3n1)[C@H](COC(C)C)CO2] | +++ | +++ | | |
| I-24 | 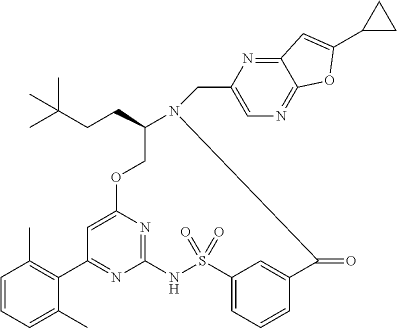 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)C)CO2] | +++ | +++ | | |
| I-25 | 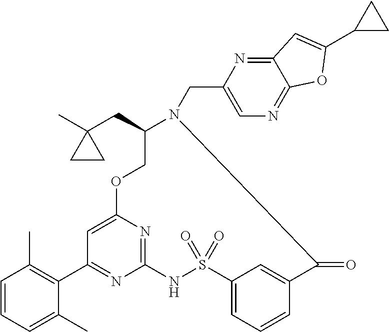 [SMILES: Cc1cccc(C)c1-c1cc2nc(n1)NS(=O)(=O)c1cccc(c1)C(=O)N(Cc1cnc3oc(C4CC4)cc3n1)[C@H](CC1(C)CC1)CO2] | +++ | | | |
| I-26 | 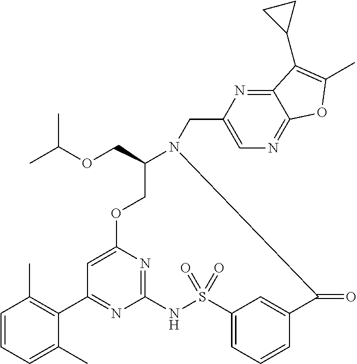 [SMILES: Cc1cccc(C)c1-c1cc2nc(n1)NS(=O)(=O)c1cccc(c1)C(=O)N(Cc1cnc3oc(C)c(C4CC4)c3n1)[C@H](COC(C)C)CO2] | +++ | | | |
| I-27 | 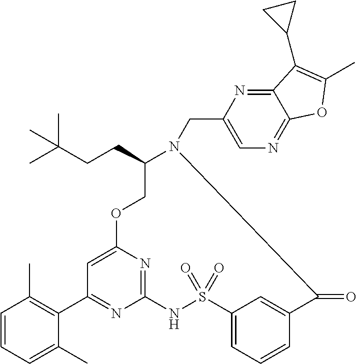 [SMILES: Cc1cccc(C)c1-c1cc2nc(n1)NS(=O)(=O)c1cccc(c1)C(=O)N(Cc1cnc3oc(C)c(C4CC4)c3n1)[C@H](CCC(C)(C)C)CO2] | +++ | | | |
| I-28 | 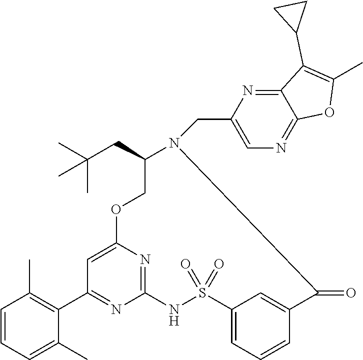 [SMILES: Cc1cccc(C)c1-c1cc2nc(n1)NS(=O)(=O)c1cccc(c1)C(=O)N(Cc1cnc3oc(C)c(C4CC4)c3n1)[C@H](CC(C)(C)C)CO2] | | | | +++ |
| I-29 | 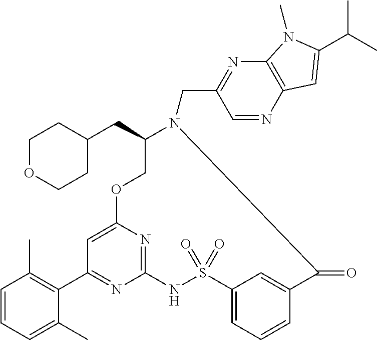 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCOCC1)CO2] | | | | ++ |
| I-30 | 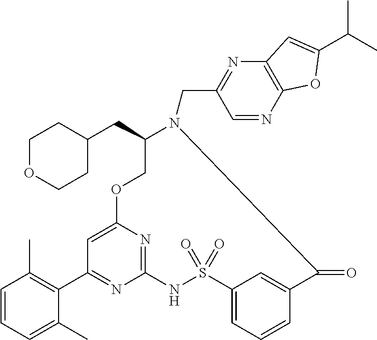 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCOCC1)CO2] | ++ | | | |
| I-32 | 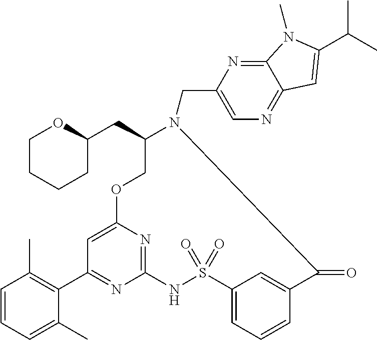 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCCO1)CO2] | +++ | | | |
| I-34 | 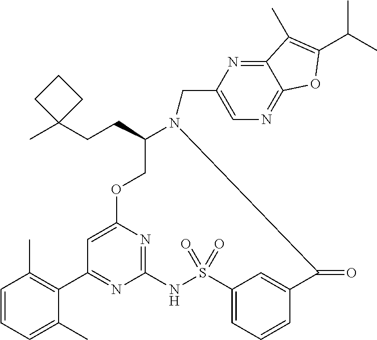 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC1(C)CCC1)CO2] | +++ | | | |
| I-35 | 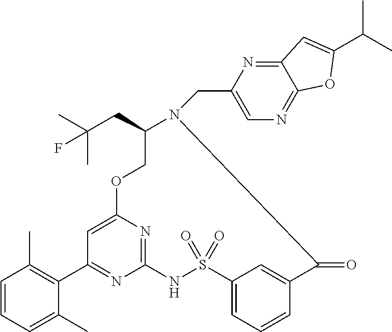 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)F)CO2] | +++ | | | |
| I-36 | 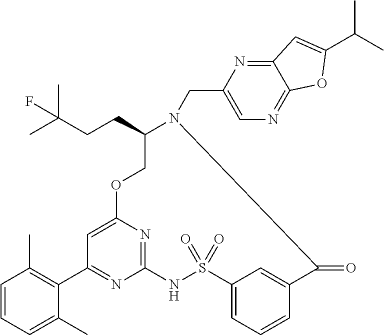 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)(C)F)CO2] | +++ | | | |
| I-38 | 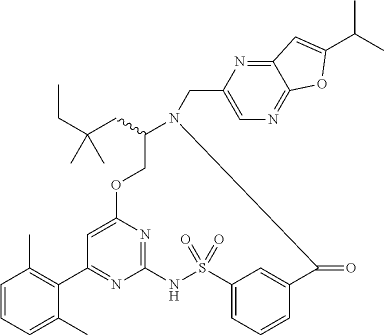 [SMILES: CCC(C)(C)CC1COc2cc(-c3c(C)cccc3C)nc(n2)NS(=O)(=O)c2cccc(c2)C(=O)N1Cc1cnc2oc(C(C)C)cc2n1] | | | | +++ |
| I-39 | 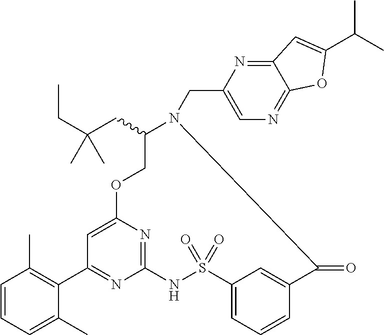 [SMILES: CCC(C)(C)CC1COc2cc(-c3c(C)cccc3C)nc(n2)NS(=O)(=O)c2cccc(c2)C(=O)N1Cc1cnc2oc(C(C)C)cc2n1] | | | | ++ |
| I-40 | 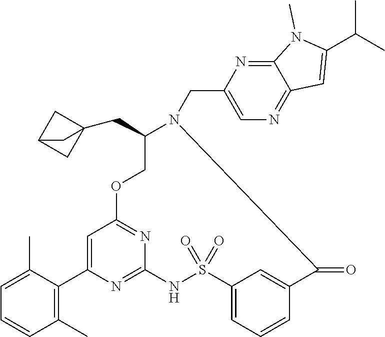 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC13CC(C1)C3)CO2] | +++ | | | |
| I-41 | 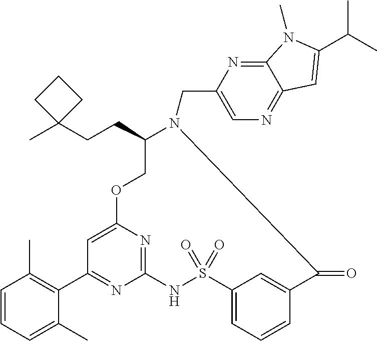 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1(C)CCC1)CO2] | +++ | | | |
| I-42 | 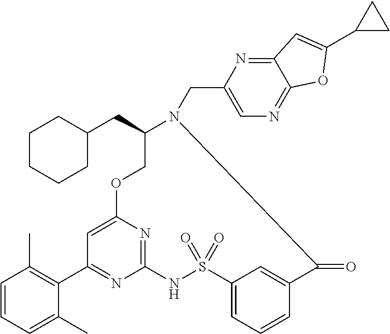 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCCC1)CO2] | +++ | | | |
| I-43 | 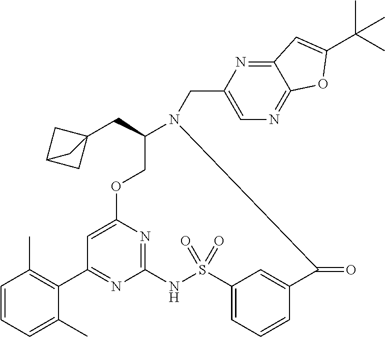 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC13CC(C1)C3)CO2] | +++ | +++ | | |
| I-44 | 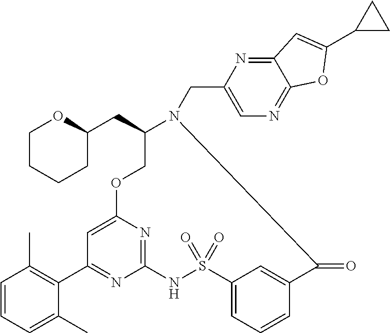 [SMILES: Cc1cccc(C)c1-c1cc2nc(n1)NS(=O)(=O)c1cccc(c1)C(=O)N(Cc1cnc3oc(C4CC4)cc3n1)[C@H](C[C@H]1CCCCO1)CO2] | +++ | | | |
| I-45 | 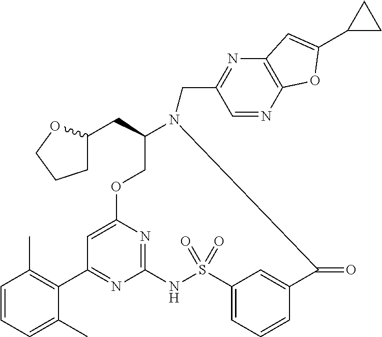 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCO1)CO2] | | | | +++ |
| I-46 | 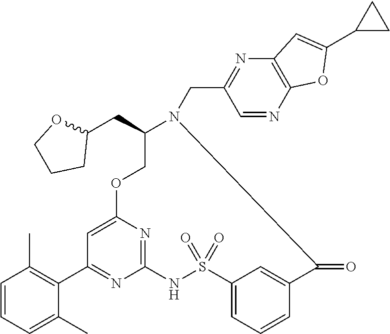 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCO1)CO2] | | | | +++ |
| I-47 | 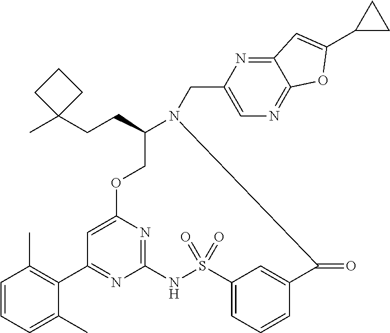 [SMILES: Cc1cccc(C)c1-c1cc2nc(n1)NS(=O)(=O)c1cccc(c1)C(=O)N(Cc1cnc3oc(C4CC4)cc3n1)[C@H](CCC1(C)CCC1)CO2] | +++ | | | |
| I-48 | 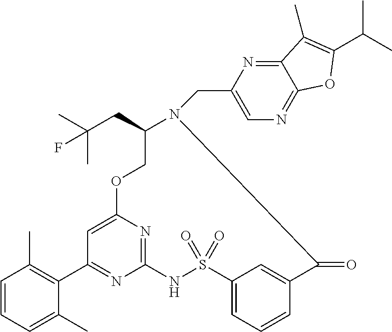 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)F)CO2] | | | | +++ |
| I-49 | 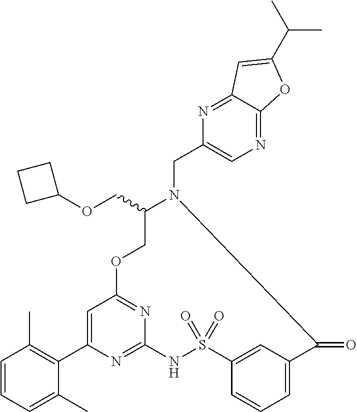 [SMILES: Cc1cccc(C)c1-c1cc2nc(n1)NS(=O)(=O)c1cccc(c1)C(=O)N(Cc1cnc3oc(C(C)C)cc3n1)C(COC1CCC1)CO2] | +++ | | | |
| I-51 | 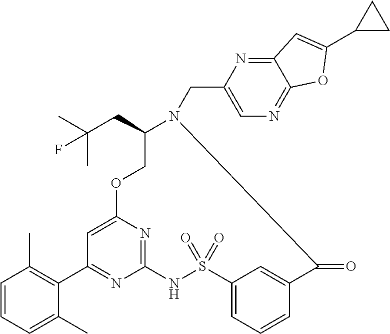 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)F)CO2] | +++ | | | |
| I-52 | 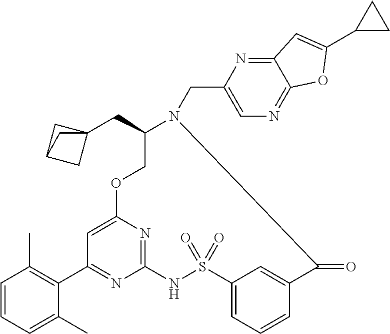 [SMILES: Cc1cccc(C)c1-c1cc2nc(n1)NS(=O)(=O)c1cccc(c1)C(=O)N(Cc1cnc3oc(C4CC4)cc3n1)[C@H](CC13CC(C1)C3)CO2] | +++ | +++ | | |
| I-53 | 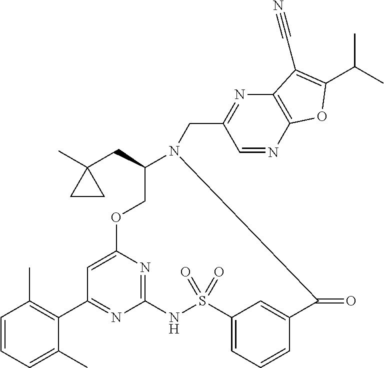 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC1(C)CC1)CO2] | +++ | | | |
| I-54 | 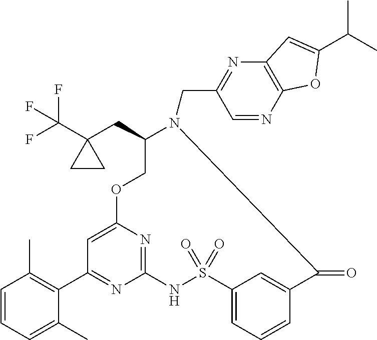 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C(F)(F)F)CC1)CO2] | +++ | | | |
| I-55 | 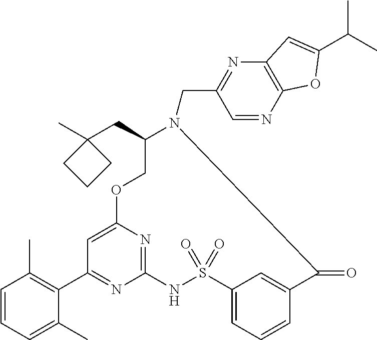 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C)CCC1)CO2] | | | | +++ |
| I-56 | 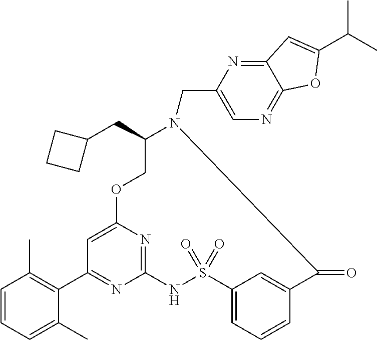 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCC1)CO2] | +++ | +++ | | |
| I-57 | 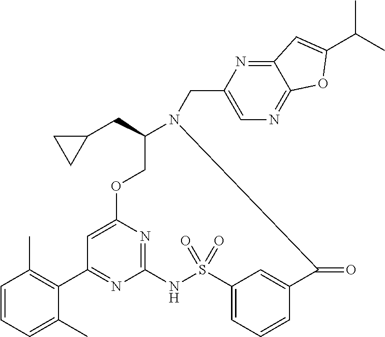 [SMILES: Cc1cccc(C)c1-c1cc2nc(n1)NS(=O)(=O)c1cccc(c1)C(=O)N(Cc1cnc3oc(C(C)C)cc3n1)[C@H](CC1CC1)CO2] | +++ | +++ | | |
| I-58 | 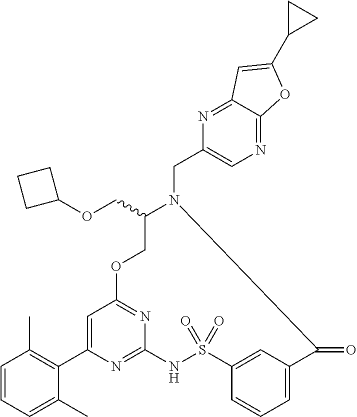 [SMILES: Cc1cccc(C)c1-c1cc2nc(n1)NS(=O)(=O)c1cccc(c1)C(=O)N(Cc1cnc3oc(C4CC4)cc3n1)C(COC1CCC1)CO2] | +++ | | | |
| I-60 | 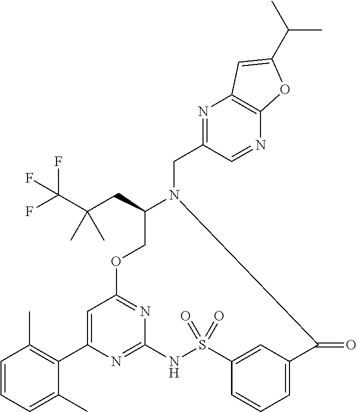 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C(F)(F)F)CO2] | | | | +++ |
| I-61 | 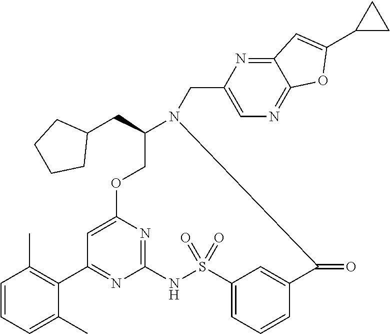 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCC1)CO2] | | +++ | | |
| I-62 | 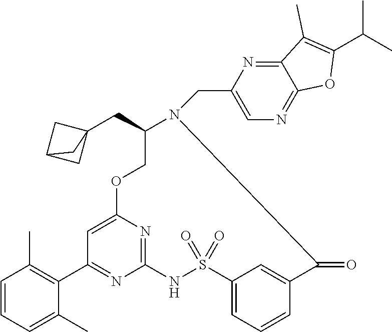 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC13CC(C1)C3)CO2] | +++ | | | |
| I-63 | 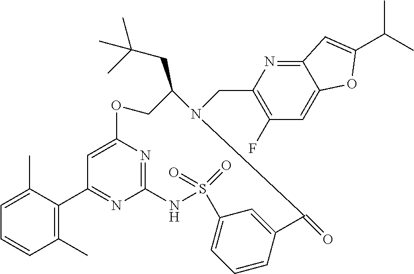 [SMILES: Cc1cccc(C)c1-c1cc2nc(n1)NS(=O)(=O)c1cccc(c1)C(=O)N(Cc1nc3cc(C(C)C)oc3cc1F)[C@H](CC(C)(C)C)CO2] | +++ | | | |
| I-64 | 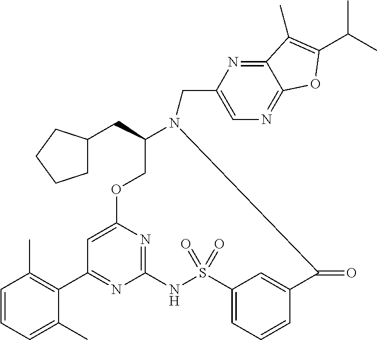 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCC1)CO2] | +++ | | | |
| I-65 | 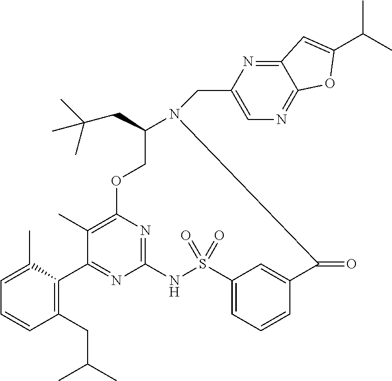 | +++ | +++ | | |
| I-66 | 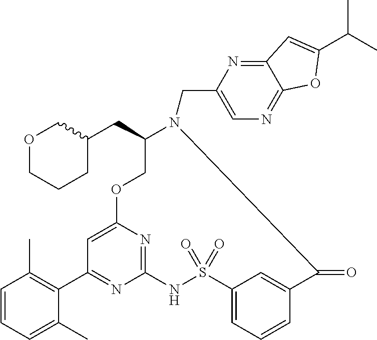 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCOC1)CO2] | | | | +++ |
| I-67 | 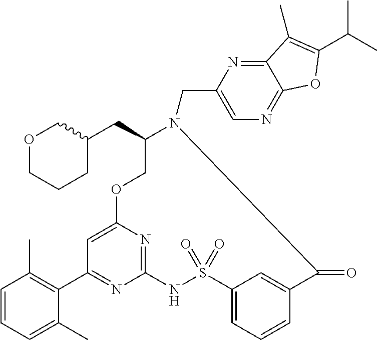 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCOC1)CO2] | +++ | | | |
| I-68 | 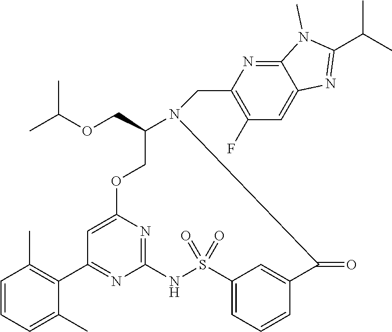 [SMILES: Cc1cccc(C)c1-c1cc2nc(n1)NS(=O)(=O)c1cccc(c1)C(=O)N(Cc1nc3c(cc1F)nc(C(C)C)n3C)[C@H](COC(C)C)CO2] | | | | +++ |
| I-69 | 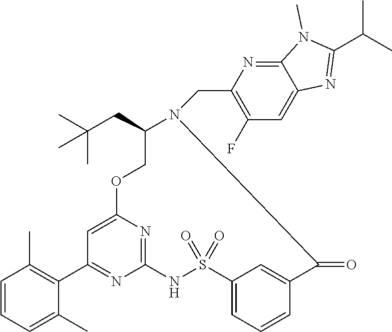 [SMILES: Cc1cccc(C)c1-c1cc2nc(n1)NS(=O)(=O)c1cccc(c1)C(=O)N(Cc1nc3c(cc1F)nc(C(C)C)n3C)[C@H](CC(C)(C)C)CO2] | +++ | | | |
| I-70 | 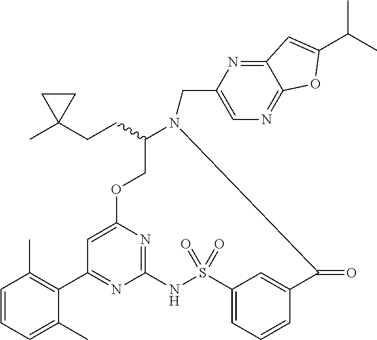 [SMILES: Cc1cccc(C)c1-c1cc2nc(n1)NS(=O)(=O)c1cccc(c1)C(=O)N(Cc1cnc3oc(C(C)C)cc3n1)C(CCC1(C)CC1)CO2] | +++ | +++ | | |
| I-71 | 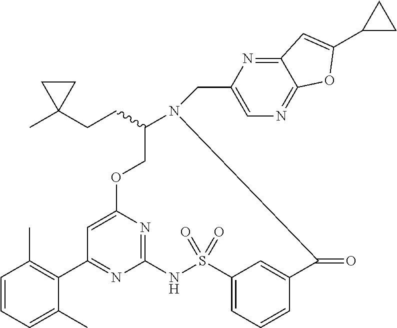 [SMILES: Cc1cccc(C)c1-c1cc2nc(n1)NS(=O)(=O)c1cccc(c1)C(=O)N(Cc1cnc3oc(C4CC4)cc3n1)C(CCC1(C)CC1)CO2] | +++ | +++ | | |
| I-72 | 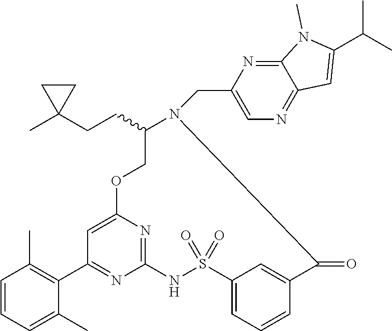 [SMILES: Cc1cccc(C)c1-c1cc2nc(n1)NS(=O)(=O)c1cccc(c1)C(=O)N(Cc1cnc3cc(C(C)C)n(C)c3n1)C(CCC1(C)CC1)CO2] | +++ | | | |
| I-73 | 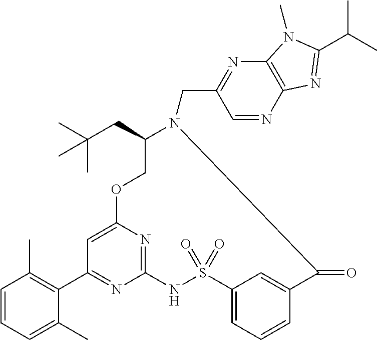 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CO2] | | | | +++ |
| I-74 | 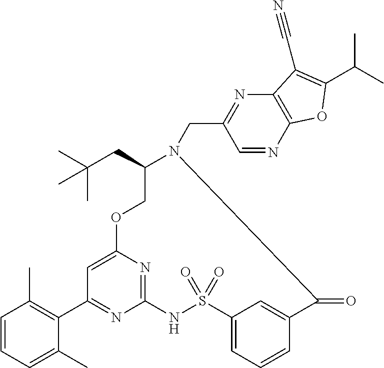 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC(C)(C)C)CO2] | | | | +++ |
| I-75 | 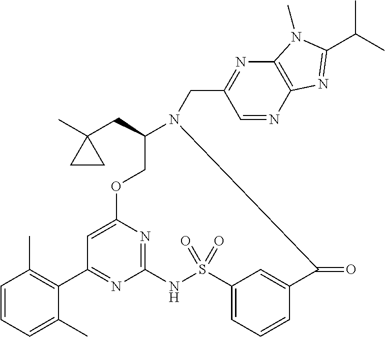 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC1(C)CC1)CO2] | +++ | | | |
| I-76 | 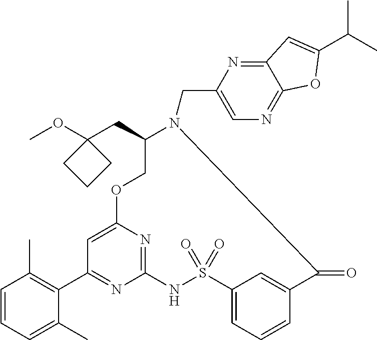 [SMILES: COC1(C[C@@H]2COc3cc(-c4c(C)cccc4C)nc(n3)NS(=O)(=O)c3cccc(c3)C(=O)N2Cc2cnc3oc(C(C)C)cc3n2)CCC1] | | | | +++ |
| I-77 | 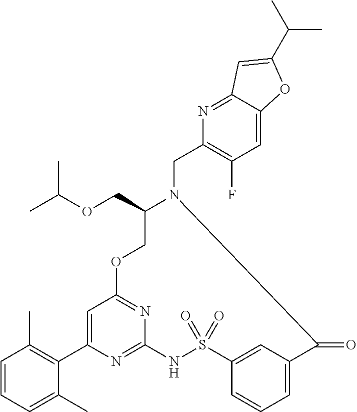 [SMILES: Cc1cccc(C)c1-c1cc2nc(n1)NS(=O)(=O)c1cccc(c1)C(=O)N(Cc1nc3cc(C(C)C)oc3cc1F)[C@H](COC(C)C)CO2] | | | | +++ |
| I-78 | 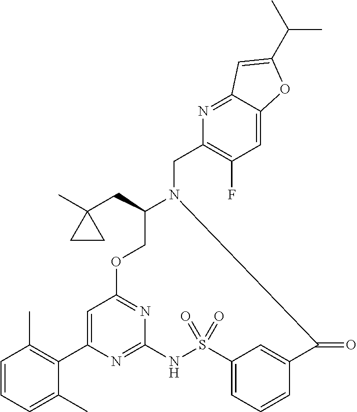 [SMILES: Cc1cccc(C)c1-c1cc2nc(n1)NS(=O)(=O)c1cccc(c1)C(=O)N(Cc1nc3cc(C(C)C)oc3cc1F)[C@H](CC1(C)CC1)CO2] | +++ | | | |
| I-79 | 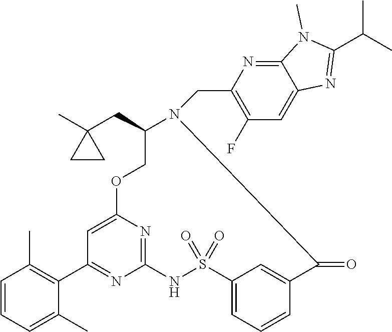 [SMILES: Cc1cccc(C)c1-c1cc2nc(n1)NS(=O)(=O)c1cccc(c1)C(=O)N(Cc1nc3c(cc1F)nc(C(C)C)n3C)[C@H](CC1(C)CC1)CO2] | | | | +++ |
| I-80 | 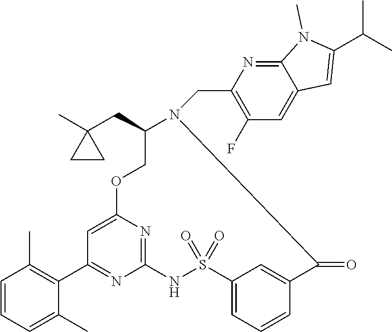 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC1(C)CC1)CO2] | +++ | | | |
| I-82 | 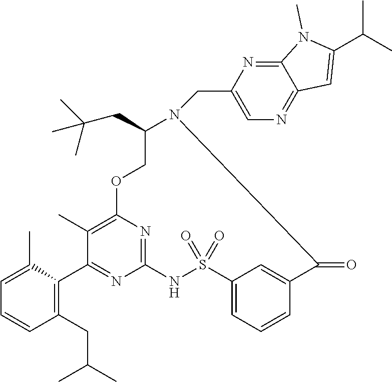 | +++ | +++ | | |
| I-84 | 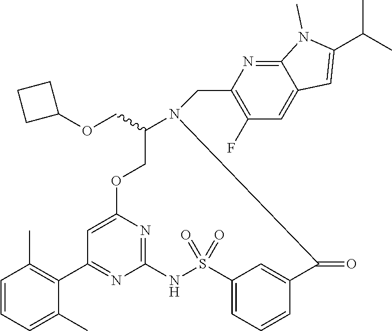 [SMILES: Cc1cccc(C)c1-c1cc2nc(n1)NS(=O)(=O)c1cccc(c1)C(=O)N(Cc1nc3c(cc1F)cc(C(C)C)n3C)C(COC1CCC1)CO2] | +++ | | | |
| I-85 | 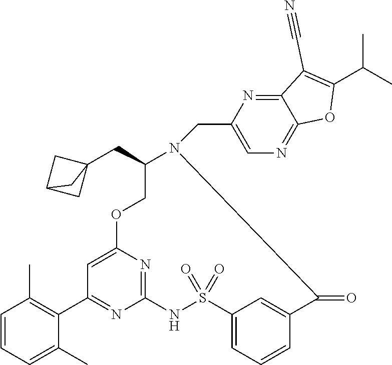 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC13CC(C1)C3)CO2] | +++ | | | |
| I-86 | 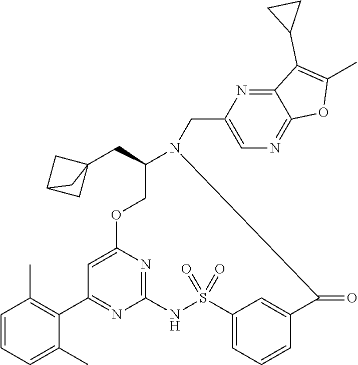 [SMILES: Cc1cccc(C)c1-c1cc2nc(n1)NS(=O)(=O)c1cccc(c1)C(=O)N(Cc1cnc3oc(C)c(C4CC4)c3n1)[C@H](CC13CC(C1)C3)CO2] | | | +++ | +++ |
| I-87 | 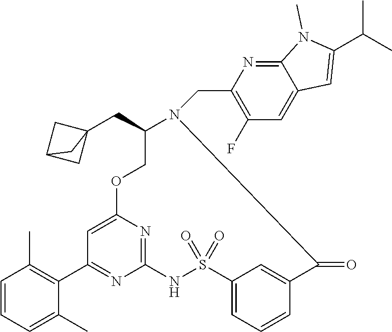 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC13CC(C1)C3)CO2] | +++ | | | |
| I-88 | 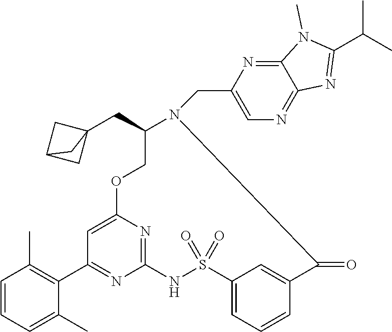 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC13CC(C1)C3)CO2] | +++ | +++ | | |
| I-89 | 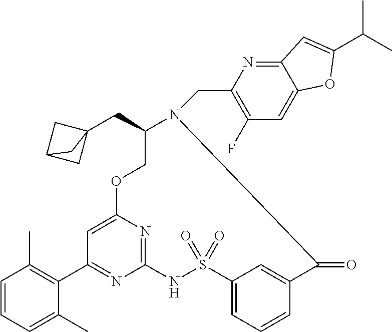 [SMILES: Cc1cccc(C)c1-c1cc2nc(n1)NS(=O)(=O)c1cccc(c1)C(=O)N(Cc1nc3cc(C(C)C)oc3cc1F)[C@H](CC13CC(C1)C3)CO2] | +++ | +++ | | |
| I-90 | 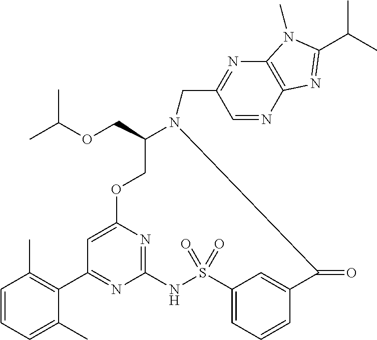 [SMILES: Cc1cccc(C)c1-c1cc2nc(n1)NS(=O)(=O)c1cccc(c1)C(=O)N(Cc1cnc3nc(C(C)C)n(C)c3n1)[C@H](COC(C)C)CO2] | +++ | | | |
| I-91 | 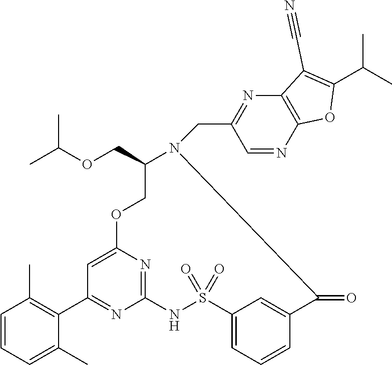 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](COC(C)C)CO2] | +++ | | | |
| I-93 | 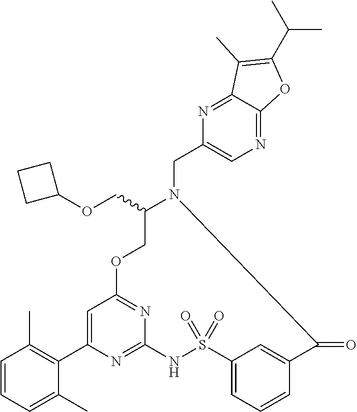 [SMILES: Cc1cccc(C)c1-c1cc2nc(n1)NS(=O)(=O)c1cccc(c1)C(=O)N(Cc1cnc3oc(C(C)C)c(C)c3n1)C(COC1CCC1)CO2] | +++ | | | |
| I-95 | 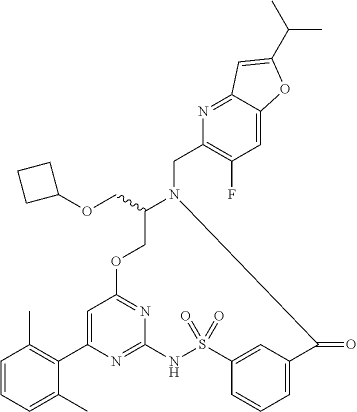 [SMILES: Cc1cccc(C)c1-c1cc2nc(n1)NS(=O)(=O)c1cccc(c1)C(=O)N(Cc1nc3cc(C(C)C)oc3cc1F)C(COC1CCC1)CO2] | +++ | | | |
| I-96 | 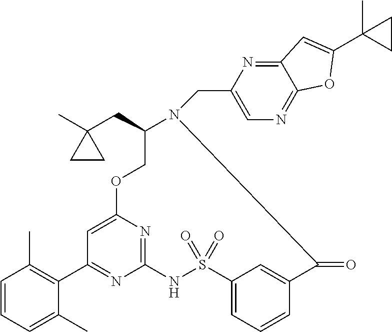 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC1(C)CC1)CO2] | +++ | +++ | | |
| I-97 | 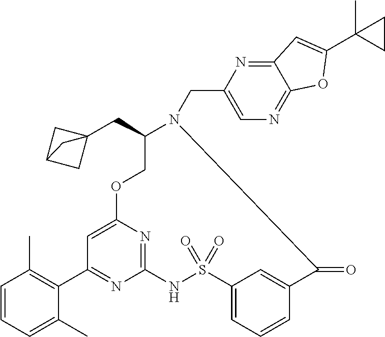 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC13CC(C1)C3)CO2] | +++ | +++ | | |
| I-98 | 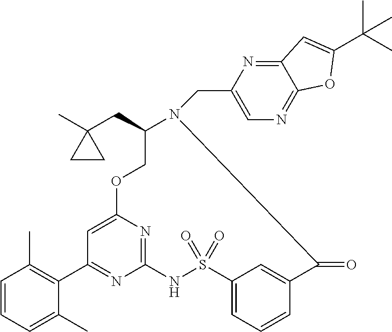 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1(C)CC1)CO2] | +++ | +++ | | |
| I-99 | 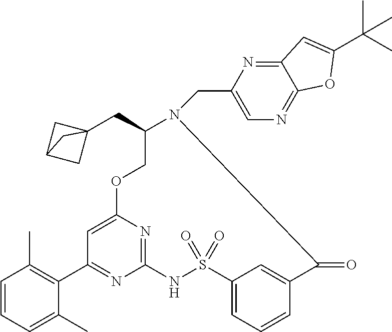 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC13CC(C1)C3)CO2] | +++ | +++ | | +++ |
| I-100 | 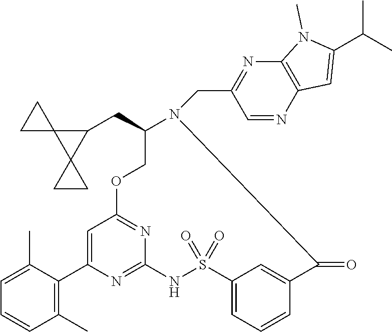 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1C3(CC3)C13CC3)CO2] | +++ | | | |
| I-101 | 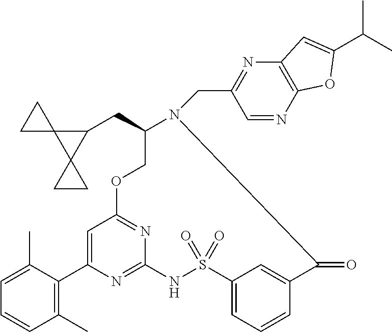 [SMILES: Cc1cccc(C)c1-c1cc2nc(n1)NS(=O)(=O)c1cccc(c1)C(=O)N(Cc1cnc3oc(C(C)C)cc3n1)[C@H](CC1C3(CC3)C13CC3)CO2] | +++ | | | |
| I-102 | 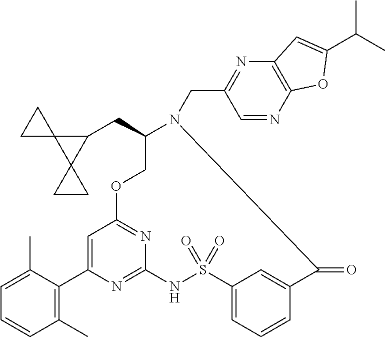 [SMILES: Cc1cccc(C)c1-c1cc2nc(n1)NS(=O)(=O)c1cccc(c1)C(=O)N(Cc1cnc3oc(C(C)C)cc3n1)[C@H](CC1C3(CC3)C13CC3)CO2] | +++ | | | |
| I-103 | 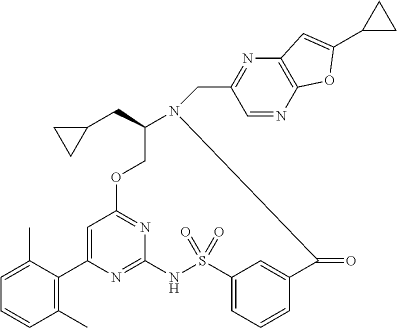 [SMILES: Cc1cccc(C)c1-c1cc2nc(n1)NS(=O)(=O)c1cccc(c1)C(=O)N(Cc1cnc3oc(C4CC4)cc3n1)[C@H](CC1CC1)CO2] | +++ | +++ | | |
| I-104 | 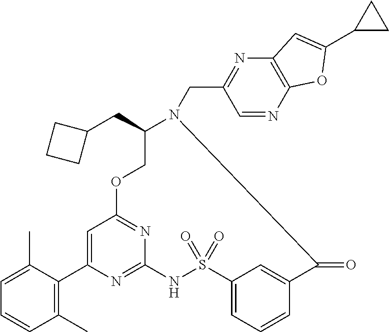 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCC1)CO2] | +++ | +++ | | |
| I-105 | 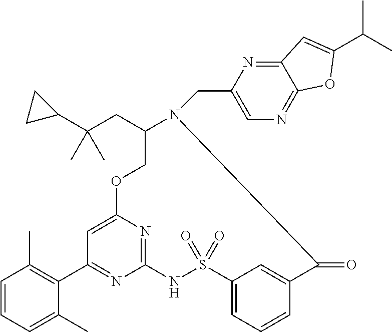 [SMILES: Cc1cccc(C)c1-c1cc2nc(n1)NS(=O)(=O)c1cccc(c1)C(=O)N(Cc1cnc3oc(C(C)C)cc3n1)C(CC(C)(C)C1CC1)CO2] | | | | +++ |
| I-106 | 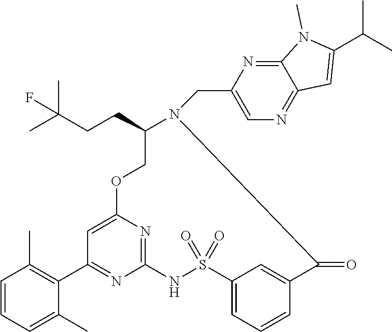 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)F)CO2] | +++ | | | |
| I-107 | 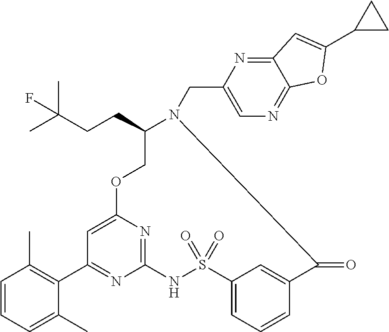 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)F)CO2] | +++ | | | |
| I-108 | 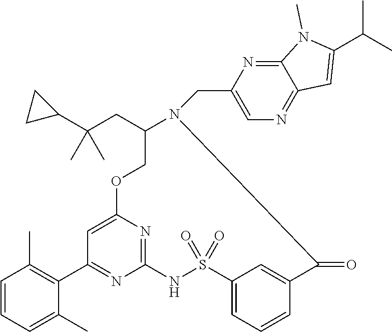 [SMILES: Cc1cccc(C)c1-c1cc2nc(n1)NS(=O)(=O)c1cccc(c1)C(=O)N(Cc1cnc3cc(C(C)C)n(C)c3n1)C(CC(C)(C)C1CC1)CO2] | | | | +++ |
| I-109 | 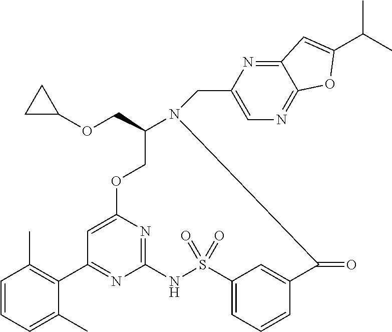 [SMILES: Cc1cccc(C)c1-c1cc2nc(n1)NS(=O)(=O)c1cccc(c1)C(=O)N(Cc1cnc3oc(C(C)C)cc3n1)[C@H](COC1CC1)CO2] | | | | +++ |
| I-110 | 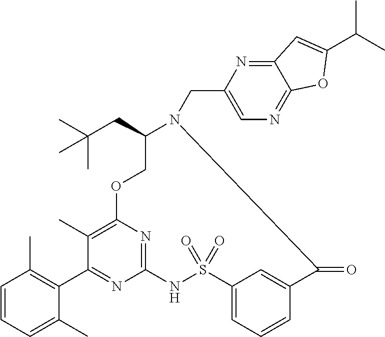 [SMILES: Cc1cccc(C)c1-c1nc2nc(c1C)OC[C@@H](CC(C)(C)C)N(Cc1cnc3oc(C(C)C)cc3n1)C(=O)c1cccc(c1)S(=O)(=O)N2] | | | +++ | +++ |
| I-111 | 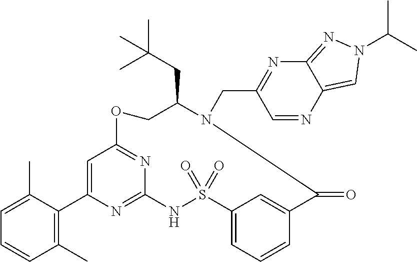 [SMILES: Cc1cccc(C)c1-c1cc2nc(n1)NS(=O)(=O)c1cccc(c1)C(=O)N(Cc1cnc3cn(C(C)C)nc3n1)[C@H](CC(C)(C)C)CO2] | | | | +++ |
| I-112 | 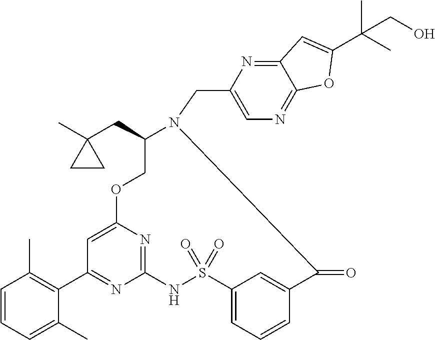 [SMILES: Cc1cccc(C)c1-c1cc2nc(n1)NS(=O)(=O)c1cccc(c1)C(=O)N(Cc1cnc3oc(C(C)(C)CO)cc3n1)[C@H](CC1(C)CC1)CO2] | | | +++ | |
| I-113 | 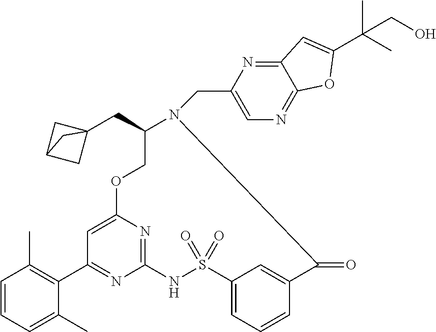 [SMILES: Cc1cccc(C)c1-c1cc2nc(n1)NS(=O)(=O)c1cccc(c1)C(=O)N(Cc1cnc3oc(C(C)(C)CO)cc3n1)[C@H](CC13CC(C1)C3)CO2] | | | +++ | +++ |
| I-114 | 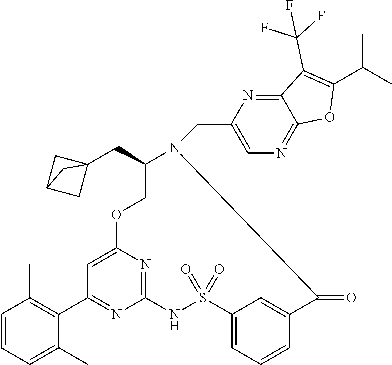 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@H](CC13CC(C1)C3)CO2] | | | | +++ |
| I-115 | 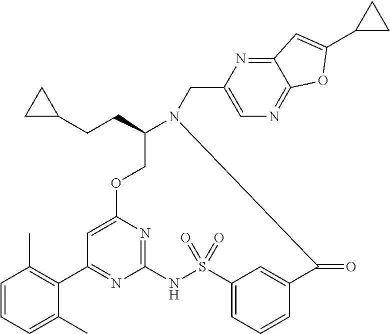 [SMILES: Cc1cccc(C)c1-c1cc2nc(n1)NS(=O)(=O)c1cccc(c1)C(=O)N(Cc1cnc3oc(C4CC4)cc3n1)[C@H](CCC1CC1)CO2] | | | | +++ |
| I-116 | 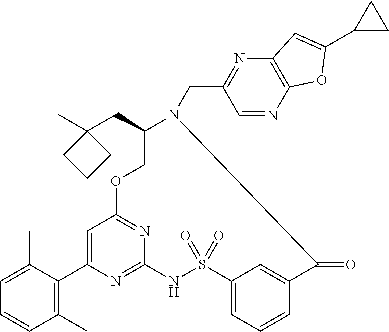 [SMILES: Cc1cccc(C)c1-c1cc2nc(n1)NS(=O)(=O)c1cccc(c1)C(=O)N(Cc1cnc3oc(C4CC4)cc3n1)[C@H](CC1(C)CCC1)CO2] | | | | +++ |
| I-117 | 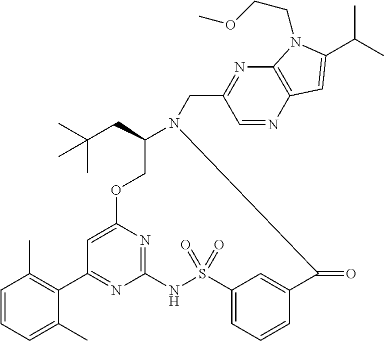 [SMILES: COCCn1c(C(C)C)cc2ncc(CN3C(=O)c4cccc(c4)S(=O)(=O)Nc4nc(cc(-c5c(C)cccc5C)n4)OC[C@H]3CC(C)(C)C)nc21] | | | | +++ |
| I-118 | 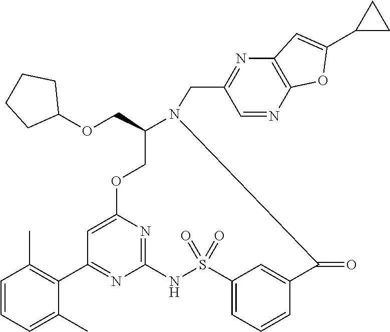 [SMILES: Cc1cccc(C)c1-c1cc2nc(n1)NS(=O)(=O)c1cccc(c1)C(=O)N(Cc1cnc3oc(C4CC4)cc3n1)[C@H](COC1CCCC1)CO2] | | +++ | | |
| I-119 | 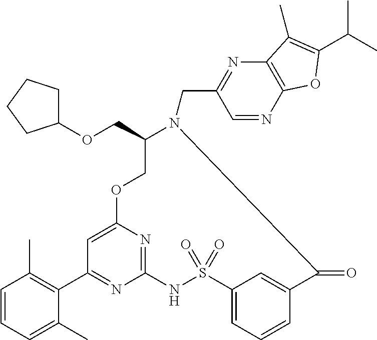 [SMILES: Cc1cccc(C)c1-c1cc2nc(n1)NS(=O)(=O)c1cccc(c1)C(=O)N(Cc1cnc3oc(C(C)C)c(C)c3n1)[C@H](COC1CCCC1)CO2] | | | | +++ |
| I-120 | 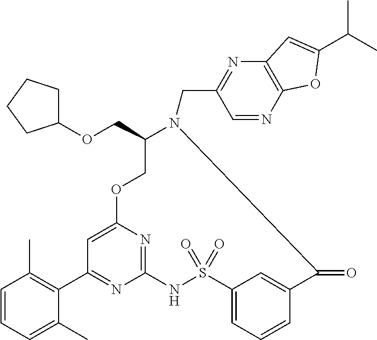 [SMILES: Cc1cccc(C)c1-c1cc2nc(n1)NS(=O)(=O)c1cccc(c1)C(=O)N(Cc1cnc3oc(C(C)C)cc3n1)[C@H](COC1CCCC1)CO2] | | | | +++ |
| I-121 | 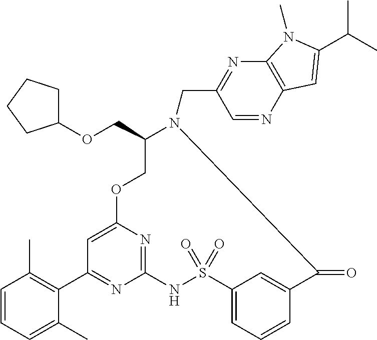 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC1CCCC1)CO2] | | | | +++ |
| I-122 | 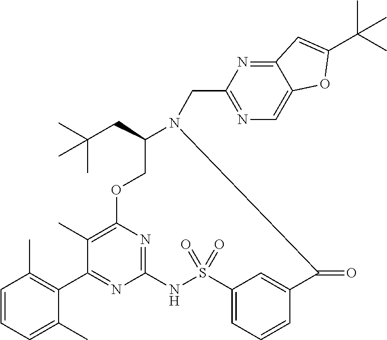 [SMILES: Cc1cccc(C)c1-c1nc2nc(c1C)OC[C@@H](CC(C)(C)C)N(Cc1ncc3oc(C(C)(C)C)cc3n1)C(=O)c1cccc(c1)S(=O)(=O)N2] | | | | +++ |
| I-123 | 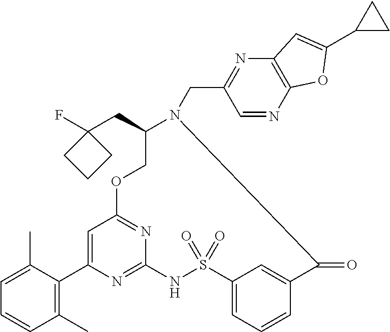 [SMILES: Cc1cccc(C)c1-c1cc2nc(n1)NS(=O)(=O)c1cccc(c1)C(=O)N(Cc1cnc3oc(C4CC4)cc3n1)[C@H](CC1(F)CCC1)CO2] | | +++ | | |
| I-124 | 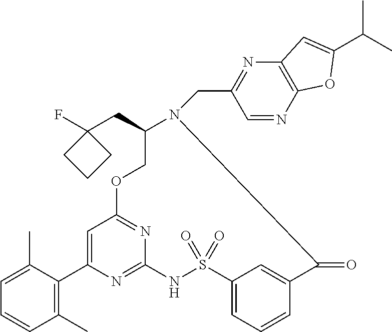 [SMILES: Cc1cccc(C)c1-c1cc2nc(n1)NS(=O)(=O)c1cccc(c1)C(=O)N(Cc1cnc3oc(C(C)C)cc3n1)[C@H](CC1(F)CCC1)CO2] | | | | +++ |
| I-125 | 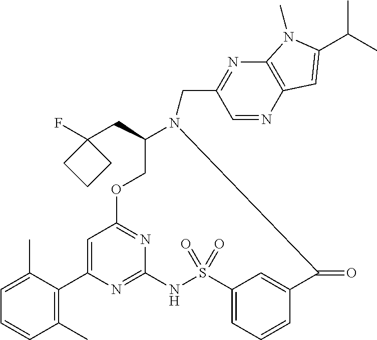 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(F)CCC1)CO2] | | | | +++ |
| I-126 | 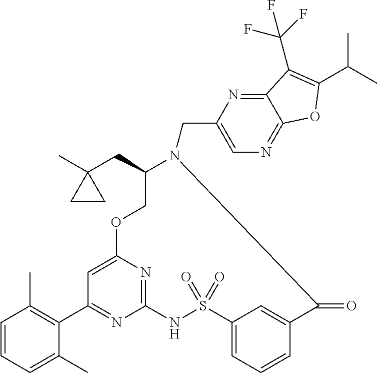 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@H](CC1(C)CC1)CO2] | | | | +++ |
| I-127 | 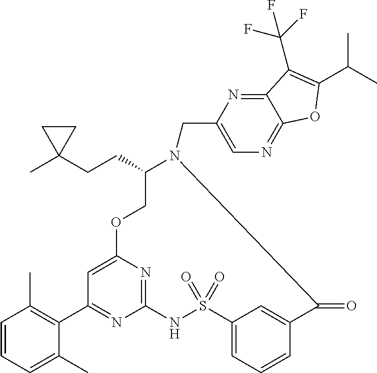 [SMILES: Cc1cccc(C)c1-c1cc2nc(n1)NS(=O)(=O)c1cccc(c1)C(=O)N(Cc1cnc3oc(C(C)C)c(C(F)(F)F)c3n1)[C@@H](CCC1(C)CC1)CO2] | | | | +++ |
| I-128 | 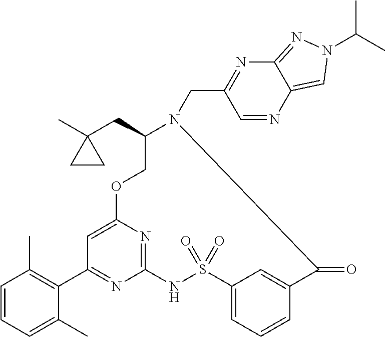 [SMILES: Cc1cccc(C)c1-c1cc2nc(n1)NS(=O)(=O)c1cccc(c1)C(=O)N(Cc1cnc3cn(C(C)C)nc3n1)[C@H](CC1(C)CC1)CO2] | | +++ | | |
| I-129 | 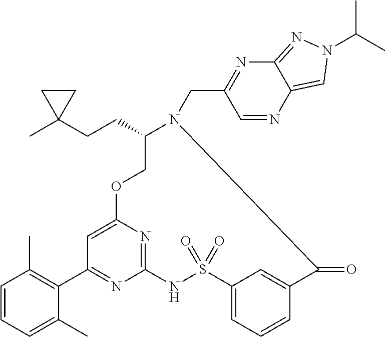 [SMILES: Cc1cccc(C)c1-c1cc2nc(n1)NS(=O)(=O)c1cccc(c1)C(=O)N(Cc1cnc3cn(C(C)C)nc3n1)[C@@H](CCC1(C)CC1)CO2] | | | | +++ |
| I-130 | 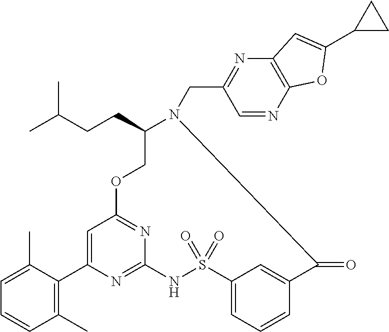 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)C)CO2] | | +++ | | |
| I-132 | 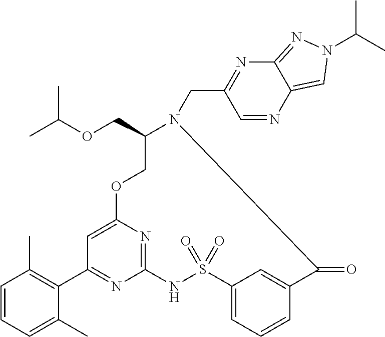 [SMILES: Cc1cccc(C)c1-c1cc2nc(n1)NS(=O)(=O)c1cccc(c1)C(=O)N(Cc1cnc3cn(C(C)C)nc3n1)[C@H](COC(C)C)CO2] | | | | +++ |
| I-133 | 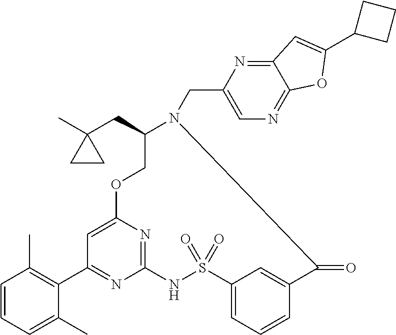 [SMILES: Cc1cccc(C)c1-c1cc2nc(n1)NS(=O)(=O)c1cccc(c1)C(=O)N(Cc1cnc3oc(C4CCC4)cc3n1)[C@H](CC1(C)CC1)CO2] | | | +++ | |
| I-134 | 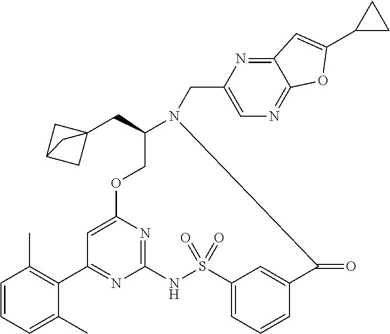 [SMILES: Cc1cccc(C)c1-c1cc2nc(n1)NS(=O)(=O)c1cccc(c1)C(=O)N(Cc1cnc3oc(C4CC4)cc3n1)[C@H](CC13CC(C1)C3)CO2] | | +++ | | |
| I-135 | 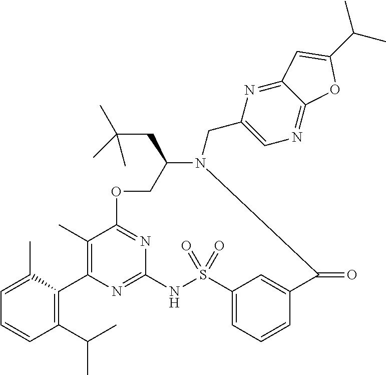 | | | | +++ |
| I-136 | 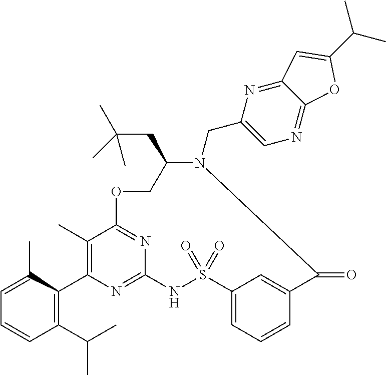 | | | | +++ |
| I-137 | 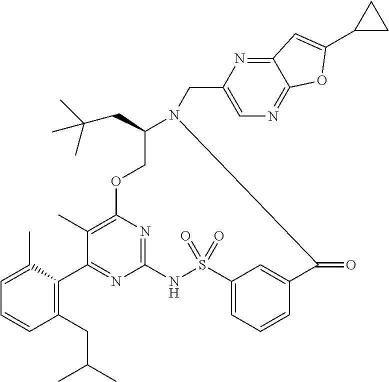 | | | +++ | +++ |
| I-138 | 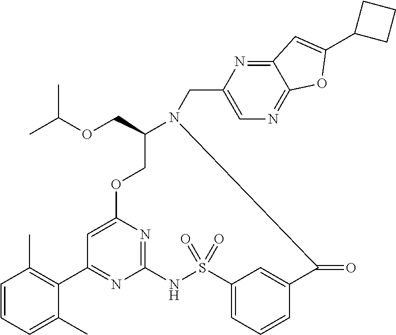 [SMILES: Cc1cccc(C)c1-c1cc2nc(n1)NS(=O)(=O)c1cccc(c1)C(=O)N(Cc1cnc3oc(C4CCC4)cc3n1)[C@H](COC(C)C)CO2] | | +++ | | |
| I-139 | 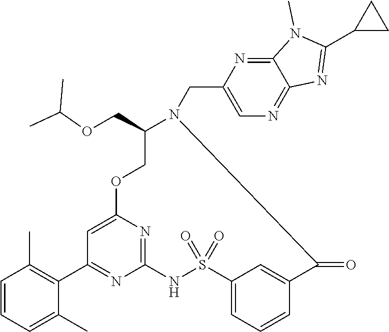 [SMILES: Cc1cccc(C)c1-c1cc2nc(n1)NS(=O)(=O)c1cccc(c1)C(=O)N(Cc1cnc3nc(C4CC4)n(C)c3n1)[C@H](COC(C)C)CO2] | | +++ | | |
| I-141 | 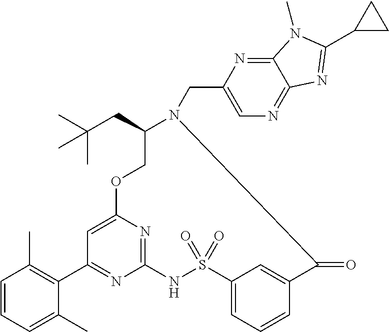 [SMILES: Cc1cccc(C)c1-c1cc2nc(n1)NS(=O)(=O)c1cccc(c1)C(=O)N(Cc1cnc3nc(C4CC4)n(C)c3n1)[C@H](CC(C)(C)C)CO2] | | | | +++ |
| I-142 | 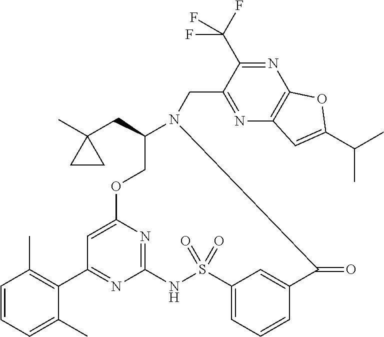 [SMILES: Cc1cccc(C)c1-c1cc2nc(n1)NS(=O)(=O)c1cccc(c1)C(=O)N(Cc1nc3cc(C(C)C)oc3nc1C(F)(F)F)[C@H](CC1(C)CC1)CO2] | | | | +++ |
| I-143 | 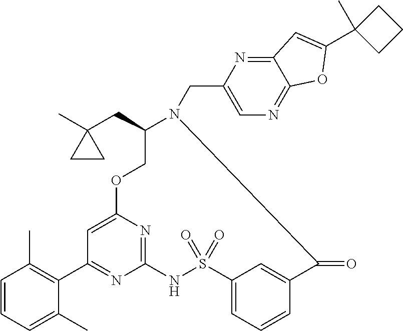 [SMILES: Cc1cccc(C)c1-c1cc2nc(n1)NS(=O)(=O)c1cccc(c1)C(=O)N(Cc1cnc3oc(C4(C)CCC4)cc3n1)[C@H](CC1(C)CC1)CO2] | | | +++ | |
| I-144 | 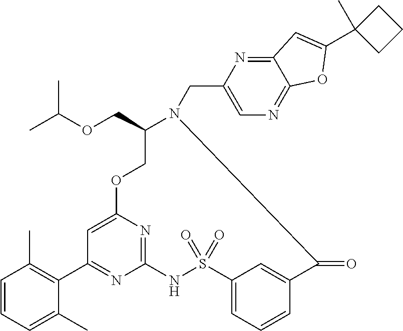 [SMILES: Cc1cccc(C)c1-c1cc2nc(n1)NS(=O)(=O)c1cccc(c1)C(=O)N(Cc1cnc3oc(C4(C)CCC4)cc3n1)[C@H](COC(C)C)CO2] | | | | +++ |
| I-145 | 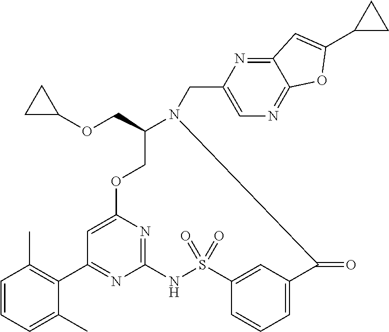 [SMILES: Cc1cccc(C)c1-c1cc2nc(n1)NS(=O)(=O)c1cccc(c1)C(=O)N(Cc1cnc3oc(C4CC4)cc3n1)[C@H](COC1CC1)CO2] | | +++ | | |
| I-146 | 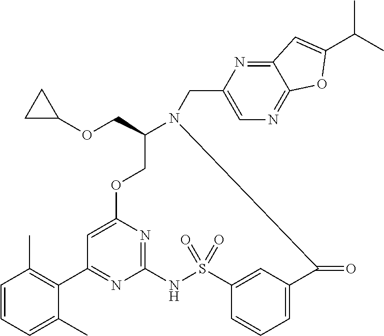 [SMILES: Cc1cccc(C)c1-c1cc2nc(n1)NS(=O)(=O)c1cccc(c1)C(=O)N(Cc1cnc3oc(C(C)C)cc3n1)[C@H](COC1CC1)CO2] | | | | +++ |
| I-147 | 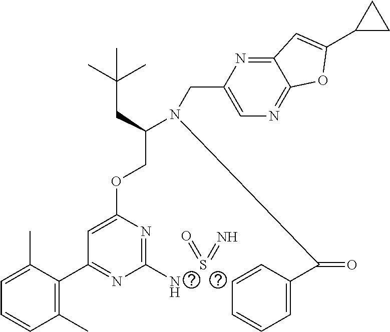 | | | | +++ |
| I-148 | 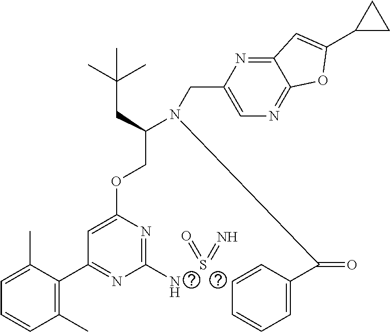 | | | | +++ |
| I-149 | 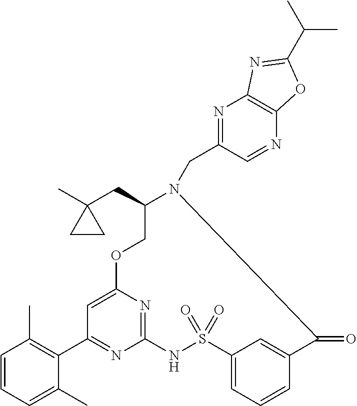 [SMILES: Cc1cccc(C)c1-c1cc2nc(n1)NS(=O)(=O)c1cccc(c1)C(=O)N(Cc1cnc3oc(C(C)C)nc3n1)[C@H](CC1(C)CC1)CO2] | | | | +++ |
| I-150 | 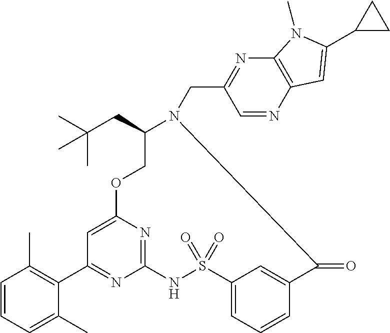 [SMILES: Cc1cccc(C)c1-c1cc2nc(n1)NS(=O)(=O)c1cccc(c1)C(=O)N(Cc1cnc3cc(C4CC4)n(C)c3n1)[C@H](CC(C)(C)C)CO2] | | | | +++ |
| I-151 | 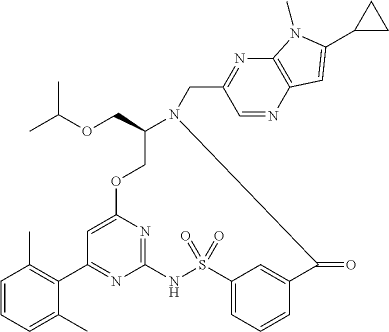 [SMILES: Cc1cccc(C)c1-c1cc2nc(n1)NS(=O)(=O)c1cccc(c1)C(=O)N(Cc1cnc3cc(C4CC4)n(C)c3n1)[C@H](COC(C)C)CO2] | | +++ | | |
| I-152 | 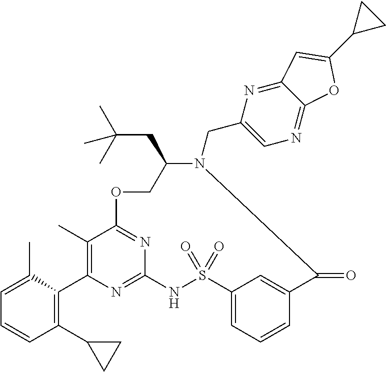 | | | +++ | +++ |
| I-153 | 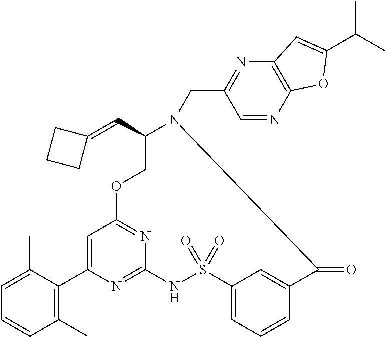 [SMILES: Cc1cccc(C)c1-c1cc2nc(n1)NS(=O)(=O)c1cccc(c1)C(=O)N(Cc1cnc3oc(C(C)C)cc3n1)[C@H](C=C1CCC1)CO2] | | | | +++ |
| I-154 | 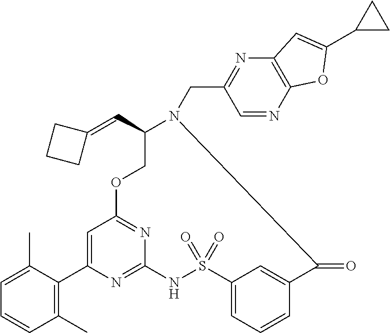 [SMILES: Cc1cccc(C)c1-c1cc2nc(n1)NS(=O)(=O)c1cccc(c1)C(=O)N(Cc1cnc3oc(C4CC4)cc3n1)[C@H](C=C1CCC1)CO2] | | | | +++ |
| I-155 | 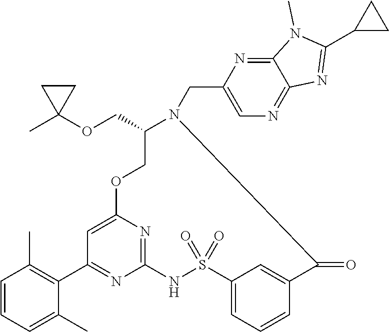 [SMILES: Cc1cccc(C)c1-c1cc2nc(n1)NS(=O)(=O)c1cccc(c1)C(=O)N(Cc1cnc3nc(C4CC4)n(C)c3n1)[C@@H](COC1(C)CC1)CO2] | | | | +++ |
| I-156 | 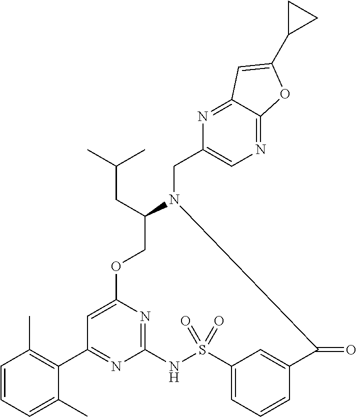 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)C)CO2] | | | | +++ |
| I-157 | 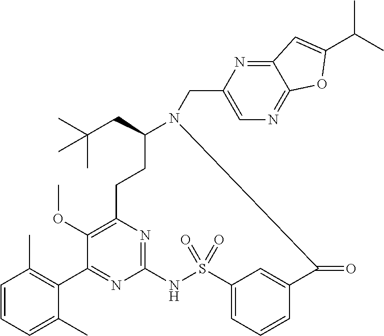 [SMILES: COc1c2nc(nc1-c1c(C)cccc1C)NS(=O)(=O)c1cccc(c1)C(=O)N(Cc1cnc3oc(C(C)C)cc3n1)[C@H](CC(C)(C)C)CC2] | | | | +++ |
| I-158 | 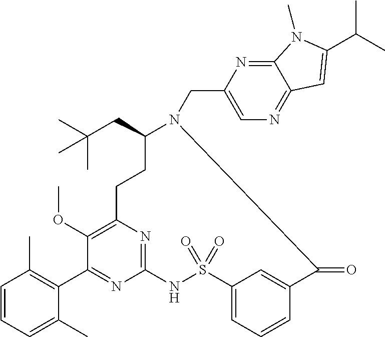 [SMILES: COc1c2nc(nc1-c1c(C)cccc1C)NS(=O)(=O)c1cccc(c1)C(=O)N(Cc1cnc3cc(C(C)C)n(C)c3n1)[C@H](CC(C)(C)C)CC2] | | | +++ | +++ |
| I-159 | 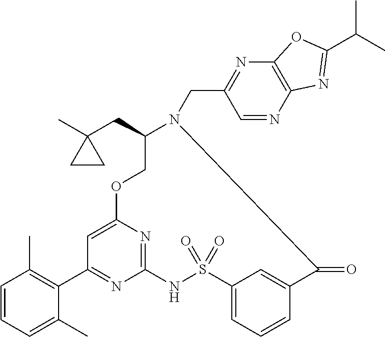 [SMILES: Cc1cccc(C)c1-c1cc2nc(n1)NS(=O)(=O)c1cccc(c1)C(=O)N(Cc1cnc3nc(C(C)C)oc3n1)[C@H](CC1(C)CC1)CO2] | | | | +++ |
| I-160 | 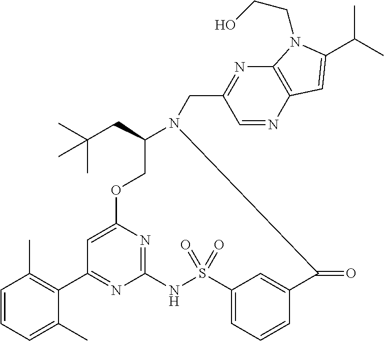 [SMILES: Cc1cccc(C)c1-c1cc2nc(n1)NS(=O)(=O)c1cccc(c1)C(=O)N(Cc1cnc3cc(C(C)C)n(CCO)c3n1)[C@H](CC(C)(C)C)CO2] | | | | +++ |
| I-161 | 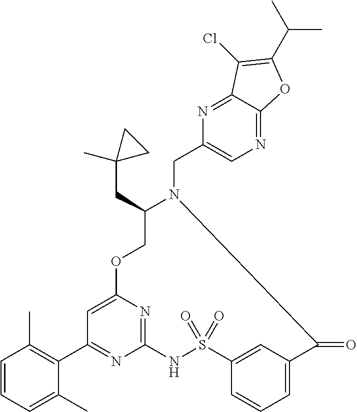 [SMILES: Cc1cccc(C)c1-c1cc2nc(n1)NS(=O)(=O)c1cccc(c1)C(=O)N(Cc1cnc3oc(C(C)C)c(Cl)c3n1)[C@H](CC1(C)CC1)CO2] | | | | +++ |
| I-162 | 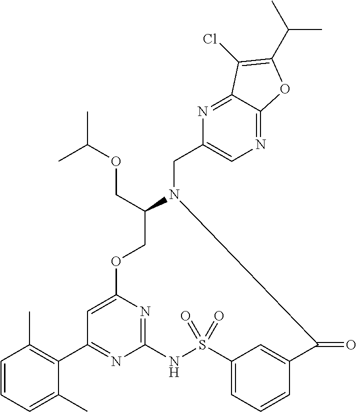 [SMILES: Cc1cccc(C)c1-c1cc2nc(n1)NS(=O)(=O)c1cccc(c1)C(=O)N(Cc1cnc3oc(C(C)C)c(Cl)c3n1)[C@H](COC(C)C)CO2] | | | | +++ |
| I-163 | 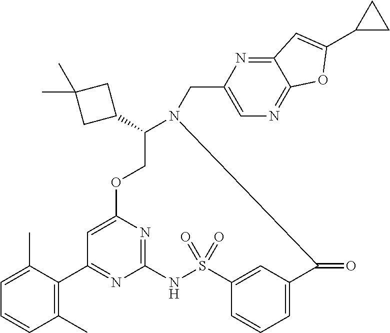 [SMILES: Cc1cccc(C)c1-c1cc2nc(n1)NS(=O)(=O)c1cccc(c1)C(=O)N(Cc1cnc3oc(C4CC4)cc3n1)[C@@H](C1CC(C)(C)C1)CO2] | | | | +++ |
| I-165 | 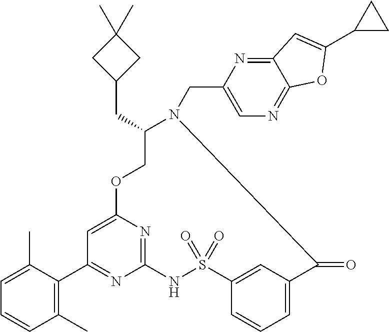 [SMILES: Cc1cccc(C)c1-c1cc2nc(n1)NS(=O)(=O)c1cccc(c1)C(=O)N(Cc1cnc3oc(C4CC4)cc3n1)[C@@H](CC1CC(C)(C)C1)CO2] | | | | +++ |
| I-166 | 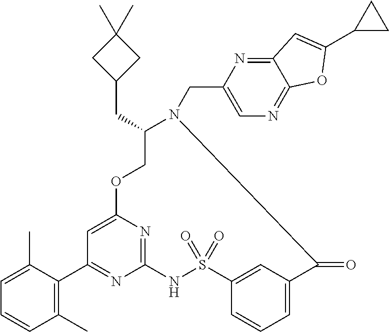 [SMILES: Cc1cccc(C)c1-c1cc2nc(n1)NS(=O)(=O)c1cccc(c1)C(=O)N(Cc1cnc3oc(C4CC4)cc3n1)[C@@H](CC1CC(C)(C)C1)CO2] | | | | ++ |
| I-167 | 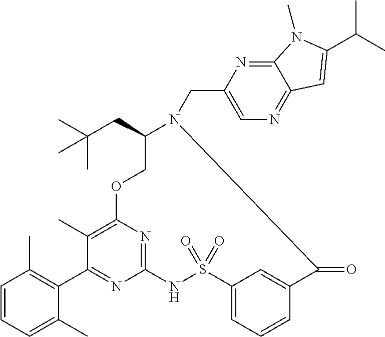 [SMILES: Cc1cccc(C)c1-c1nc2nc(c1C)OC[C@@H](CC(C)(C)C)N(Cc1cnc3cc(C(C)C)n(C)c3n1)C(=O)c1cccc(c1)S(=O)(=O)N2] | | +++ | | |
| I-168 | 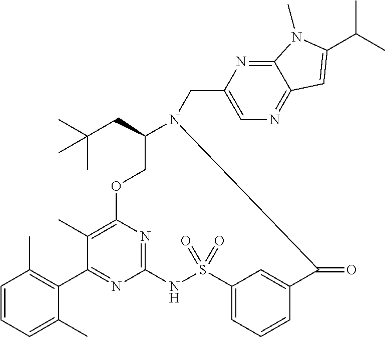 [SMILES: Cc1cccc(C)c1-c1nc2nc(c1C)OC[C@@H](CC(C)(C)C)N(Cc1cnc3cc(C(C)C)n(C)c3n1)C(=O)c1cccc(c1)S(=O)(=O)N2] | | | +++ | +++ |
| I-169 | 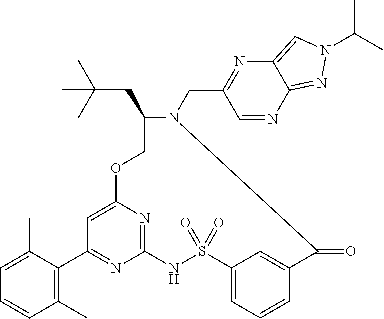 [SMILES: Cc1cccc(C)c1-c1cc2nc(n1)NS(=O)(=O)c1cccc(c1)C(=O)N(Cc1cnc3nn(C(C)C)cc3n1)[C@H](CC(C)(C)C)CO2] | | | | +++ |
| I-170 | 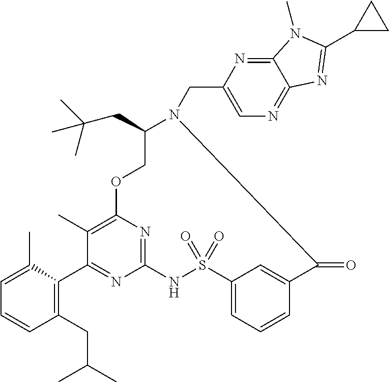 | | | +++ | +++ |
| I-171 | 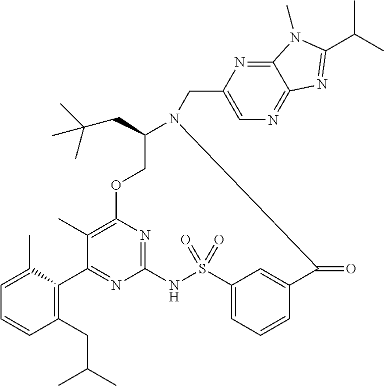 | | | +++ | +++ |
| I-172 | 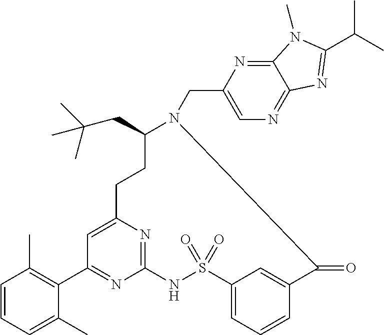 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CC2] | | | | +++ |
| I-173 | 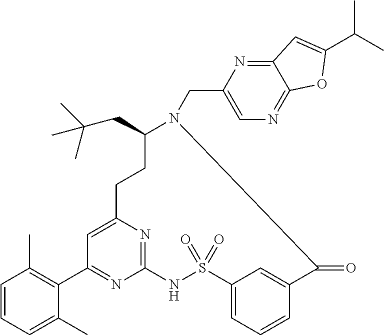 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C)CC2] | | | | +++ |
| I-174 | 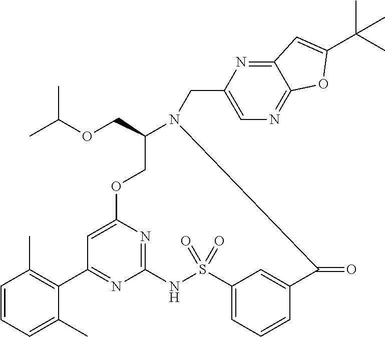 [SMILES: Cc1cccc(C)c1-c1cc2nc(n1)NS(=O)(=O)c1cccc(c1)C(=O)N(Cc1cnc3oc(C(C)(C)C)cc3n1)[C@H](COC(C)C)CO2] | | | +++ | |
| I-175 | 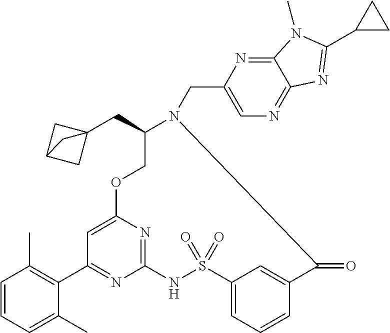 [SMILES: Cc1cccc(C)c1-c1cc2nc(n1)NS(=O)(=O)c1cccc(c1)C(=O)N(Cc1cnc3nc(C4CC4)n(C)c3n1)[C@H](CC13CC(C1)C3)CO2] | | | | +++ |
| I-177 | 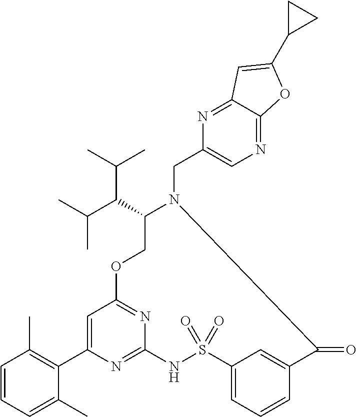 [SMILES: Cc1cccc(C)c1-c1cc2nc(n1)NS(=O)(=O)c1cccc(c1)C(=O)N(Cc1cnc3oc(C4CC4)cc3n1)[C@@H](C(C(C)C)C(C)C)CO2] | | | +++ | +++ |
| I-178 | 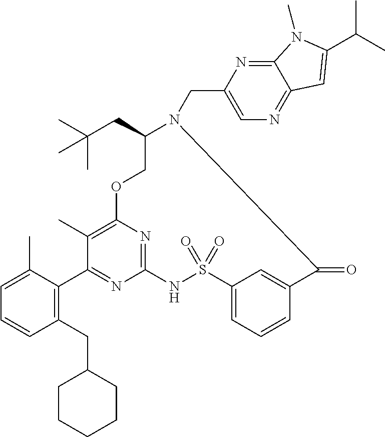 [SMILES: Cc1cccc(CC2CCCCC2)c1-c1nc2nc(c1C)OC[C@@H](CC(C)(C)C)N(Cc1cnc3cc(C(C)C)n(C)c3n1)C(=O)c1cccc(c1)S(=O)(=O)N2] | | | +++ | |
| I-179 | 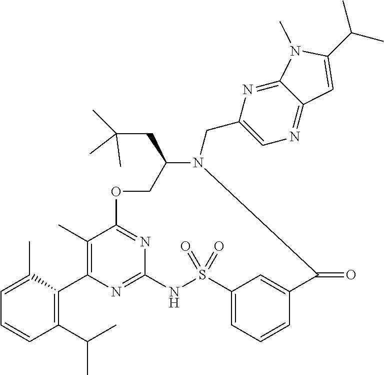 | | | +++ | |
| I-180 | 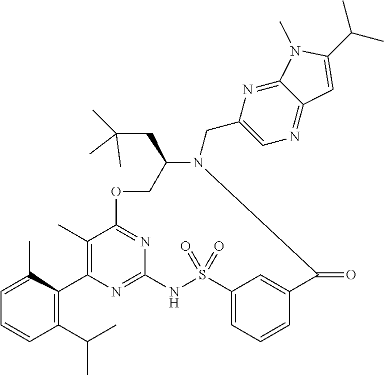 | | | | +++ |
| I-181 | 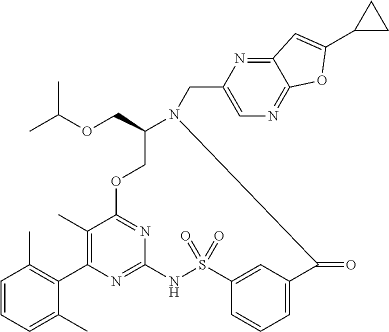 [SMILES: Cc1cccc(C)c1-c1nc2nc(c1C)OC[C@@H](COC(C)C)N(Cc1cnc3oc(C4CC4)cc3n1)C(=O)c1cccc(c1)S(=O)(=O)N2] | | | | +++ |
| I-182 | 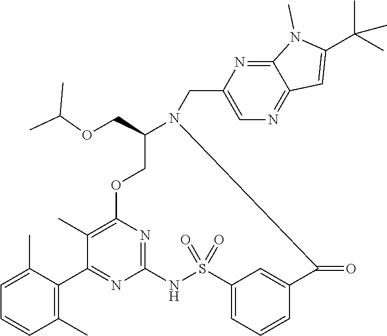 [SMILES: Cc1cccc(C)c1-c1nc2nc(c1C)OC[C@@H](COC(C)C)N(Cc1cnc3cc(C(C)(C)C)n(C)c3n1)C(=O)c1cccc(c1)S(=O)(=O)N2] | | +++ | +++ | +++ |
| I-183 | 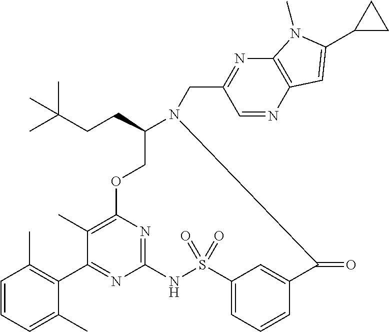 [SMILES: Cc1cccc(C)c1-c1nc2nc(c1C)OC[C@@H](CCC(C)(C)C)N(Cc1cnc3cc(C4CC4)n(C)c3n1)C(=O)c1cccc(c1)S(=O)(=O)N2] | | | | +++ |
| I-184 | 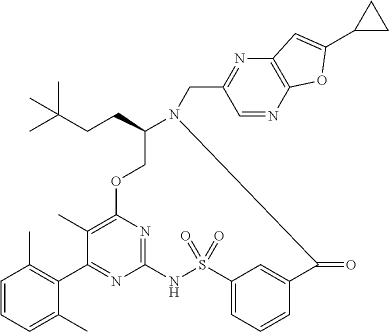 [SMILES: Cc1cccc(C)c1-c1nc2nc(c1C)OC[C@@H](CCC(C)(C)C)N(Cc1cnc3oc(C4CC4)cc3n1)C(=O)c1cccc(c1)S(=O)(=O)N2] | | | | +++ |
| I-185 | 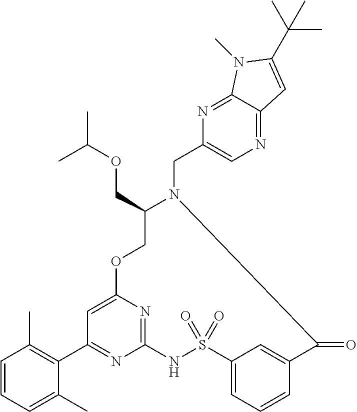 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](COC(C)C)CO2] | | | +++ | +++ |
| I-186 | 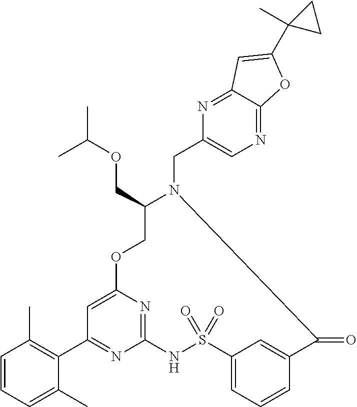 [SMILES: Cc1cccc(C)c1-c1cc2nc(n1)NS(=O)(=O)c1cccc(c1)C(=O)N(Cc1cnc3oc(C4(C)CC4)cc3n1)[C@H](COC(C)C)CO2] | | | | +++ |
| I-187 | 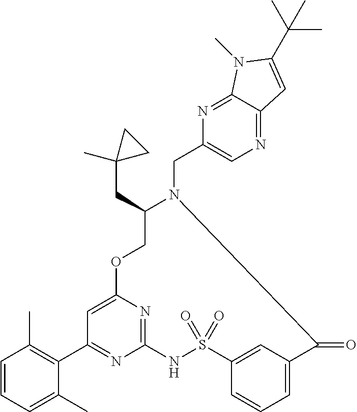 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](CC1(C)CC1)CO2] | | | +++ | +++ |
| I-188 | 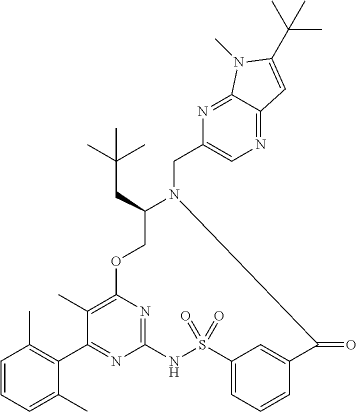 [SMILES: Cc1cccc(C)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2] | | +++ | +++ | +++ |
| I-189 | 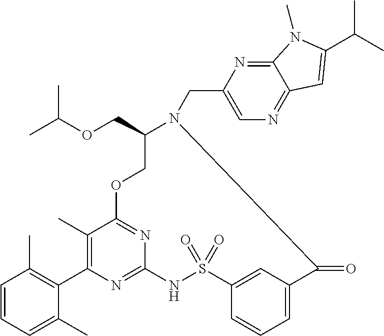 [SMILES: Cc1cccc(C)c1-c1nc2nc(c1C)OC[C@@H](COC(C)C)N(Cc1cnc3cc(C(C)C)n(C)c3n1)C(=O)c1cccc(c1)S(=O)(=O)N2] | | | | +++ |
| I-190 | 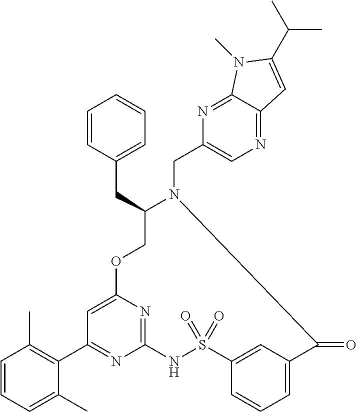 [SMILES: Cc1cccc(C)c1-c1cc2nc(n1)NS(=O)(=O)c1cccc(c1)C(=O)N(Cc1cnc3cc(C(C)C)n(C)c3n1)[C@H](Cc1ccccc1)CO2] | | | | +++ |
| I-191 | 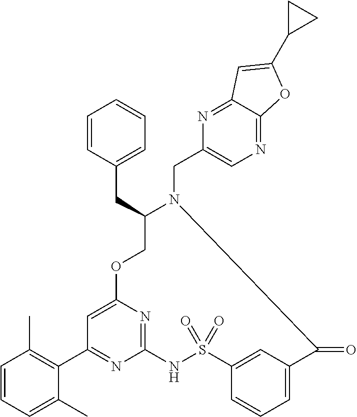 [SMILES: Cc1cccc(C)c1-c1cc2nc(n1)NS(=O)(=O)c1cccc(c1)C(=O)N(Cc1cnc3oc(C4CC4)cc3n1)[C@H](Cc1ccccc1)CO2] | | | | +++ |
| I-192 | 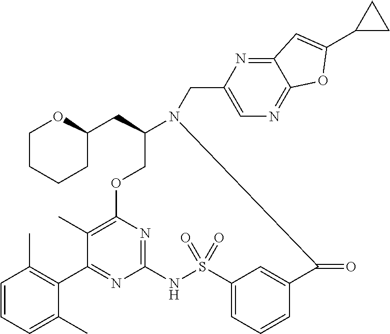 [SMILES: Cc1cccc(C)c1-c1nc2nc(c1C)OC[C@@H](C[C@H]1CCCCO1)N(Cc1cnc3oc(C4CC4)cc3n1)C(=O)c1cccc(c1)S(=O)(=O)N2] | | | | +++ |
| I-193 | 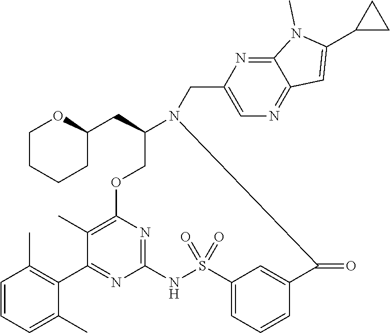 [SMILES: Cc1cccc(C)c1-c1nc2nc(c1C)OC[C@@H](C[C@H]1CCCCO1)N(Cc1cnc3cc(C4CC4)n(C)c3n1)C(=O)c1cccc(c1)S(=O)(=O)N2] | | | +++ | +++ |
| I-194 | 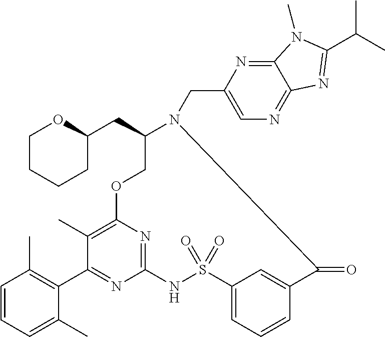 [SMILES: Cc1cccc(C)c1-c1nc2nc(c1C)OC[C@@H](C[C@H]1CCCCO1)N(Cc1cnc3nc(C(C)C)n(C)c3n1)C(=O)c1cccc(c1)S(=O)(=O)N2] | | | +++ | +++ |
| I-195 | 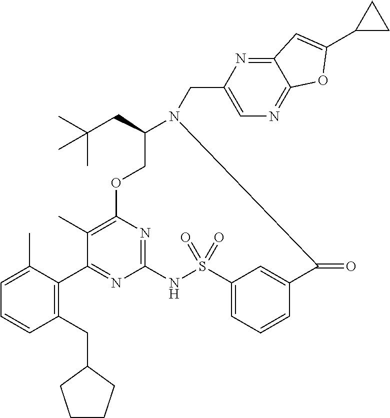 [SMILES: Cc1cccc(CC2CCCC2)c1-c1nc2nc(c1C)OC[C@@H](CC(C)(C)C)N(Cc1cnc3oc(C4CC4)cc3n1)C(=O)c1cccc(c1)S(=O)(=O)N2] | | | +++ | +++ |
| I-196 | 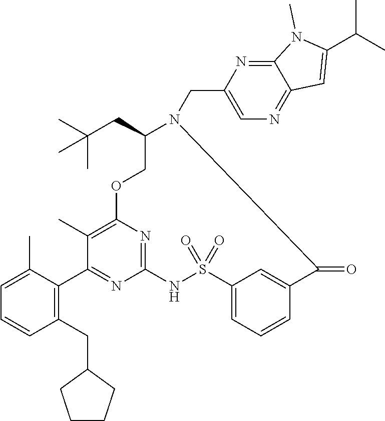 [SMILES: Cc1cccc(CC2CCCC2)c1-c1nc2nc(c1C)OC[C@@H](CC(C)(C)C)N(Cc1cnc3cc(C(C)C)n(C)c3n1)C(=O)c1cccc(c1)S(=O)(=O)N2] | | | +++ | |
| I-197 | 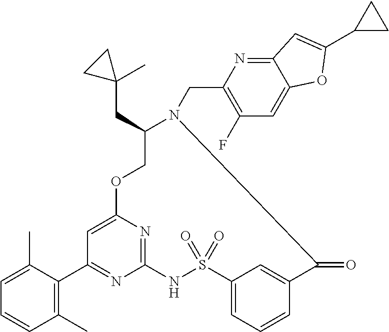 [SMILES: Cc1cccc(C)c1-c1cc2nc(n1)NS(=O)(=O)c1cccc(c1)C(=O)N(Cc1nc3cc(C4CC4)oc3cc1F)[C@H](CC1(C)CC1)CO2] | | | +++ | |
| I-198 | 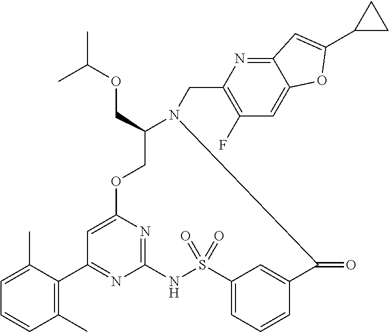 [SMILES: Cc1cccc(C)c1-c1cc2nc(n1)NS(=O)(=O)c1cccc(c1)C(=O)N(Cc1nc3cc(C4CC4)oc3cc1F)[C@H](COC(C)C)CO2] | | | +++ | |
| I-199 | 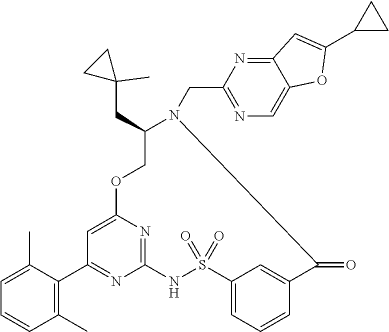 [SMILES: Cc1cccc(C)c1-c1cc2nc(n1)NS(=O)(=O)c1cccc(c1)C(=O)N(Cc1ncc3oc(C4CC4)cc3n1)[C@H](CC1(C)CC1)CO2] | | | +++ | |
| I-200 | 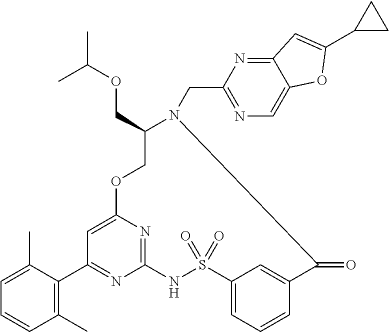 [SMILES: Cc1cccc(C)c1-c1cc2nc(n1)NS(=O)(=O)c1cccc(c1)C(=O)N(Cc1ncc3oc(C4CC4)cc3n1)[C@H](COC(C)C)CO2] | | | | +++ |
| I-202 | 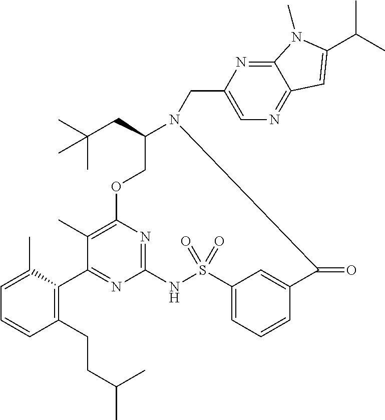 | | | +++ | |
| I-204 | 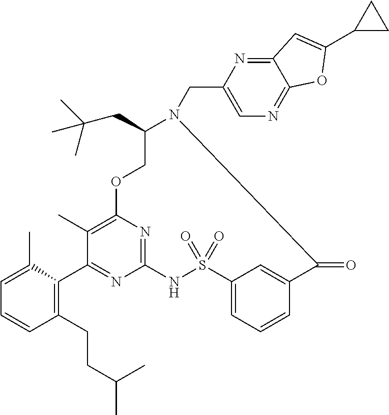 | | | +++ | +++ |
| I-205 | 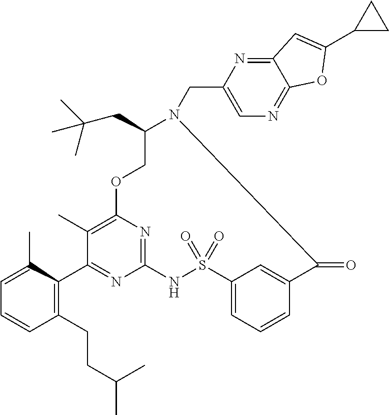 | | | | +++ |
| I-206 | 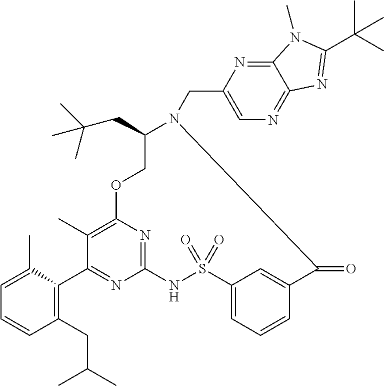 | | +++ | +++ | |
| I-207 | 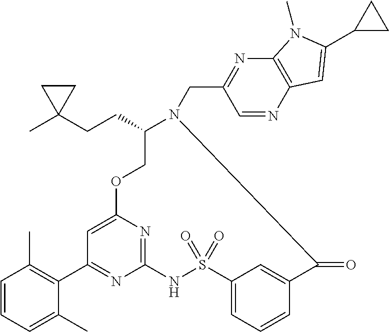 [SMILES: Cc1cccc(C)c1-c1cc2nc(n1)NS(=O)(=O)c1cccc(c1)C(=O)N(Cc1cnc3cc(C4CC4)n(C)c3n1)[C@@H](CCC1(C)CC1)CO2] | | | +++ | +++ |
| I-208 | 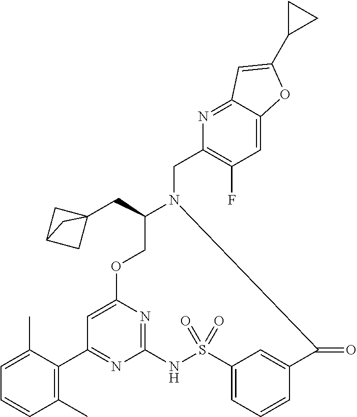 [SMILES: Cc1cccc(C)c1-c1cc2nc(n1)NS(=O)(=O)c1cccc(c1)C(=O)N(Cc1nc3cc(C4CC4)oc3cc1F)[C@H](CC13CC(C1)C3)CO2] | | | +++ | |
| I-209 | 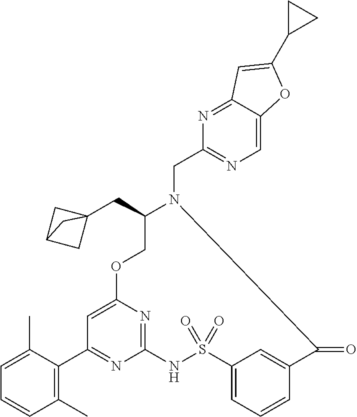 [SMILES: Cc1cccc(C)c1-c1cc2nc(n1)NS(=O)(=O)c1cccc(c1)C(=O)N(Cc1ncc3oc(C4CC4)cc3n1)[C@H](CC13CC(C1)C3)CO2] | | | +++ | |
| I-210 | 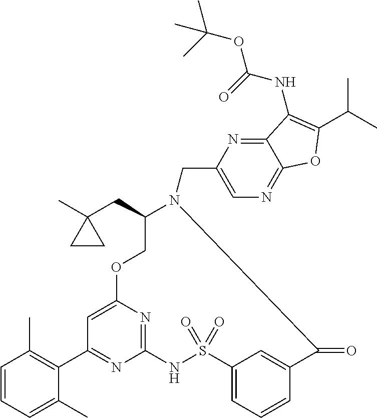 [SMILES: Cc1cccc(C)c1-c1cc2nc(n1)NS(=O)(=O)c1cccc(c1)C(=O)N(Cc1cnc3oc(C(C)C)c(NC(=O)OC(C)(C)C)c3n1)[C@H](CC1(C)CC1)CO2] | | | | +++ |
| I-211 | 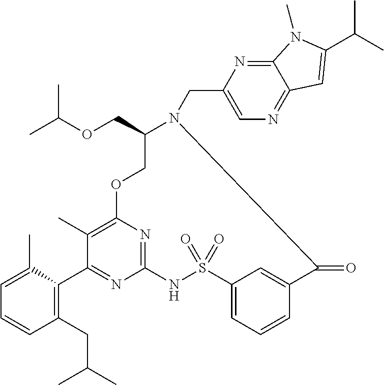 | | | +++ | +++ |
| I-213 | 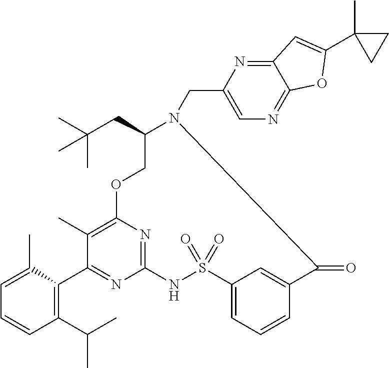 | | | +++ | +++ |
| I-215 | 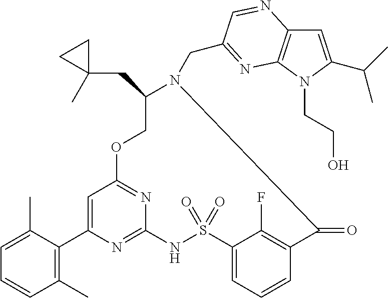 [SMILES: Cc1cccc(C)c1-c1cc2nc(n1)NS(=O)(=O)c1cccc(c1F)C(=O)N(Cc1cnc3cc(C(C)C)n(CCO)c3n1)[C@H](CC1(C)CC1)CO2] | | | | +++ |
| I-216 | 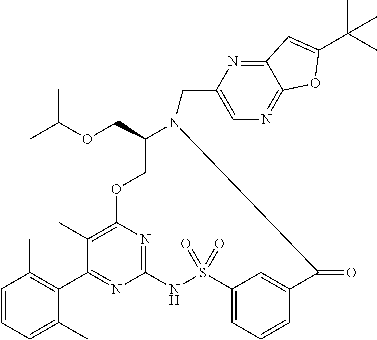 [SMILES: Cc1cccc(C)c1-c1nc2nc(c1C)OC[C@@H](COC(C)C)N(Cc1cnc3oc(C(C)(C)C)cc3n1)C(=O)c1cccc(c1)S(=O)(=O)N2] | | | | +++ |
| I-217 | 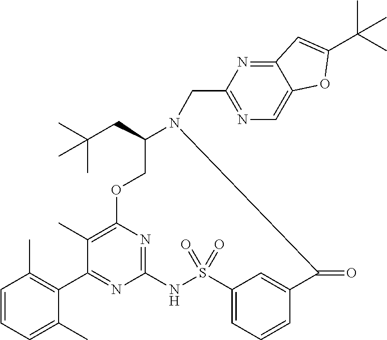 [SMILES: Cc1cccc(C)c1-c1nc2nc(c1C)OC[C@@H](CC(C)(C)C)N(Cc1ncc3oc(C(C)(C)C)cc3n1)C(=O)c1cccc(c1)S(=O)(=O)N2] | | | +++ | +++ |
| I-218 | 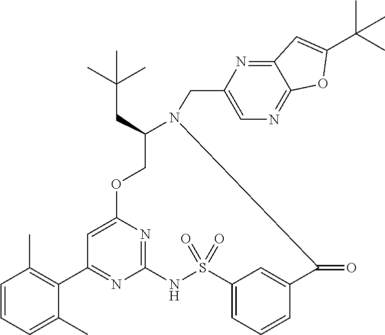 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC(C)(C)C)CO2] | | | +++ | +++ |
| I-219 | 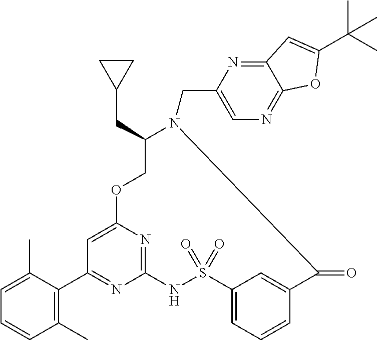 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CC1)CO2] | | | | +++ |
| I-220 | 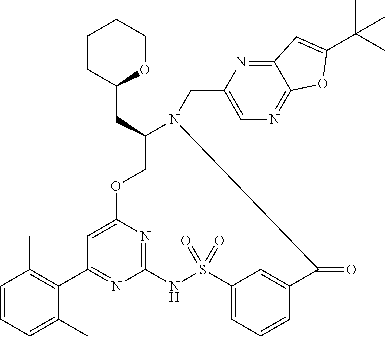 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@H]1CCCCO1)CO2] | | | | +++ |
| I-221 | 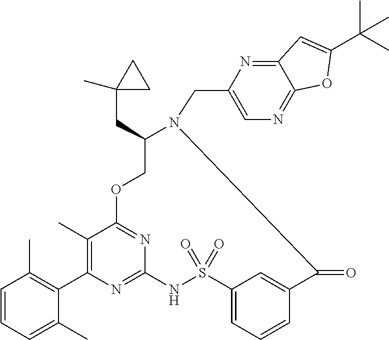 [SMILES: Cc1cccc(C)c1-c1nc2nc(c1C)OC[C@@H](CC1(C)CC1)N(Cc1cnc3oc(C(C)(C)C)cc3n1)C(=O)c1cccc(c1)S(=O)(=O)N2] | | | | +++ |
| I-222 | 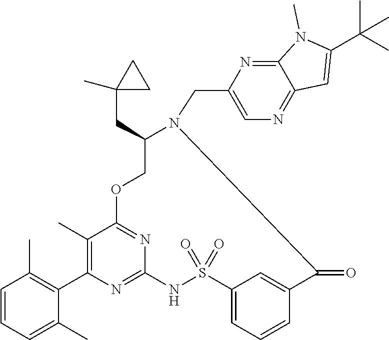 [SMILES: Cc1cccc(C)c1-c1nc2nc(c1C)OC[C@@H](CC1(C)CC1)N(Cc1cnc3cc(C(C)(C)C)n(C)c3n1)C(=O)c1cccc(c1)S(=O)(=O)N2] | | | +++ | +++ |
| I-223 | 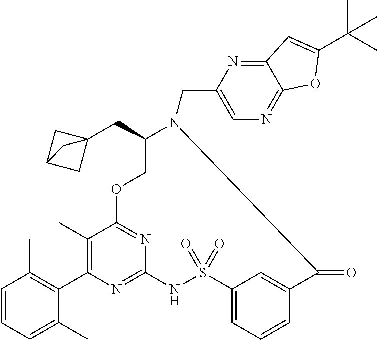 [SMILES: Cc1cccc(C)c1-c1nc2nc(c1C)OC[C@@H](CC13CC(C1)C3)N(Cc1cnc3oc(C(C)(C)C)cc3n1)C(=O)c1cccc(c1)S(=O)(=O)N2] | | | | +++ |
| I-224 | 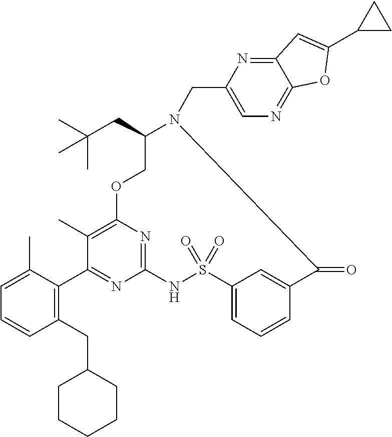 [SMILES: Cc1cccc(CC2CCCCC2)c1-c1nc2nc(c1C)OC[C@@H](CC(C)(C)C)N(Cc1cnc3oc(C4CC4)cc3n1)C(=O)c1cccc(c1)S(=O)(=O)N2] | | | | +++ |
| I-225 | 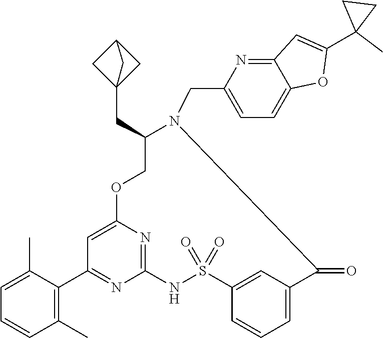 [SMILES: Cc1cccc(C)c1-c1cc2nc(n1)NS(=O)(=O)c1cccc(c1)C(=O)N(Cc1ccc3oc(C4(C)CC4)cc3n1)[C@H](CC13CC(C1)C3)CO2] | | | | +++ |
| I-226 | 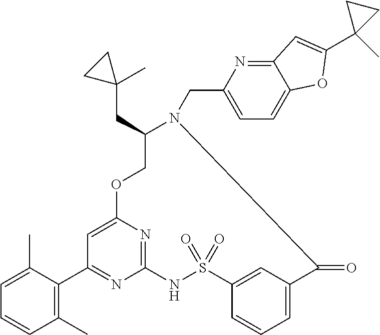 [SMILES: Cc1cccc(C)c1-c1cc2nc(n1)NS(=O)(=O)c1cccc(c1)C(=O)N(Cc1ccc3oc(C4(C)CC4)cc3n1)[C@H](CC1(C)CC1)CO2] | | | | +++ |
| I-227 | 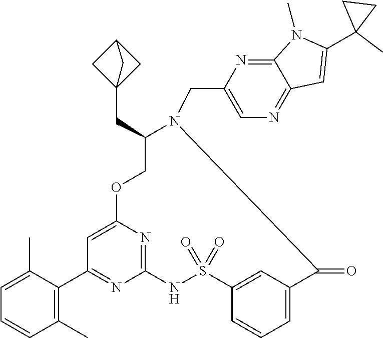 [SMILES: Cc1cccc(C)c1-c1cc2nc(n1)NS(=O)(=O)c1cccc(c1)C(=O)N(Cc1cnc3cc(C4(C)CC4)n(C)c3n1)[C@H](CC13CC(C1)C3)CO2] | | | +++ | +++ |
| I-228 | 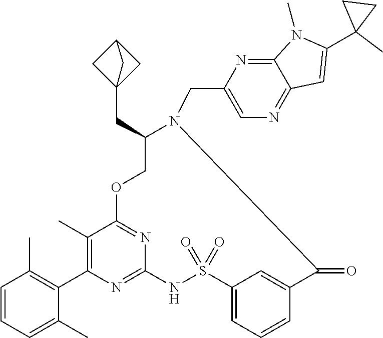 [SMILES: Cc1cccc(C)c1-c1nc2nc(c1C)OC[C@@H](CC13CC(C1)C3)N(Cc1cnc3cc(C4(C)CC4)n(C)c3n1)C(=O)c1cccc(c1)S(=O)(=O)N2] | | | +++ | +++ |
| I-229 | 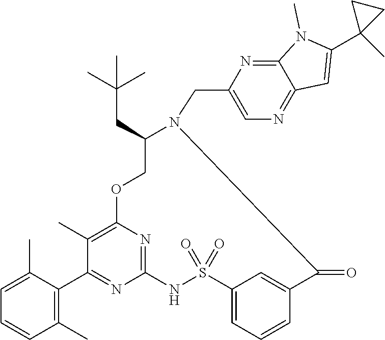 [SMILES: Cc1cccc(C)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] | | | | +++ |
| I-230 | 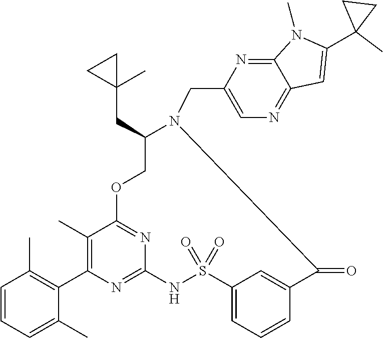 [SMILES: Cc1cccc(C)c1-c1nc2nc(c1C)OC[C@@H](CC1(C)CC1)N(Cc1cnc3cc(C4(C)CC4)n(C)c3n1)C(=O)c1cccc(c1)S(=O)(=O)N2] | | | | +++ |
| I-231 | 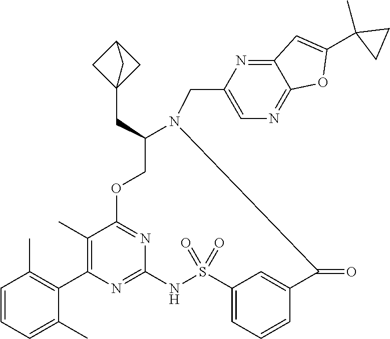 [SMILES: Cc1cccc(C)c1-c1nc2nc(c1C)OC[C@@H](CC13CC(C1)C3)N(Cc1cnc3oc(C4(C)CC4)cc3n1)C(=O)c1cccc(c1)S(=O)(=O)N2] | | | +++ | +++ |
| I-232 | 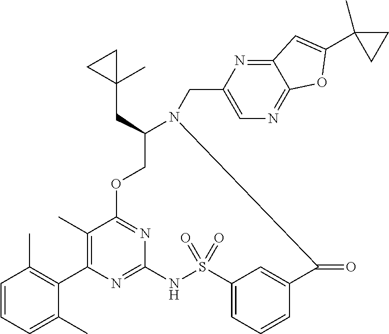 [SMILES: Cc1cccc(C)c1-c1nc2nc(c1C)OC[C@@H](CC1(C)CC1)N(Cc1cnc3oc(C4(C)CC4)cc3n1)C(=O)c1cccc(c1)S(=O)(=O)N2] | | | | +++ |
| I-233 | 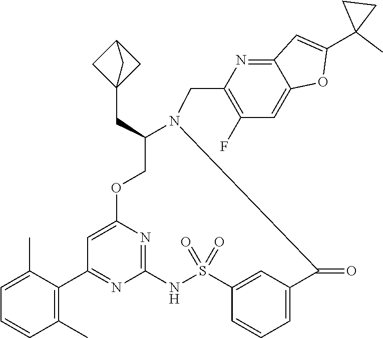 [SMILES: Cc1cccc(C)c1-c1cc2nc(n1)NS(=O)(=O)c1cccc(c1)C(=O)N(Cc1nc3cc(C4(C)CC4)oc3cc1F)[C@H](CC13CC(C1)C3)CO2] | | | | +++ |
| I-234 | 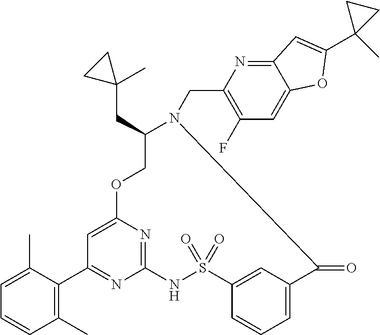 [SMILES: Cc1cccc(C)c1-c1cc2nc(n1)NS(=O)(=O)c1cccc(c1)C(=O)N(Cc1nc3cc(C4(C)CC4)oc3cc1F)[C@H](CC1(C)CC1)CO2] | | | | +++ |
| I-235 | 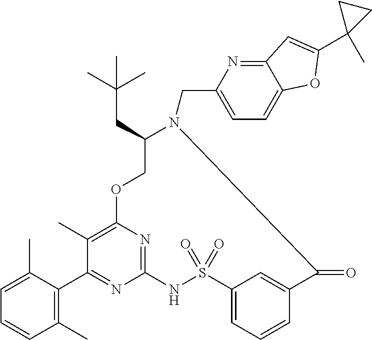 [SMILES: Cc1cccc(C)c1-c1nc2nc(c1C)OC[C@@H](CC(C)(C)C)N(Cc1ccc3oc(C4(C)CC4)cc3n1)C(=O)c1cccc(c1)S(=O)(=O)N2] | | | | +++ |
| I-236 | 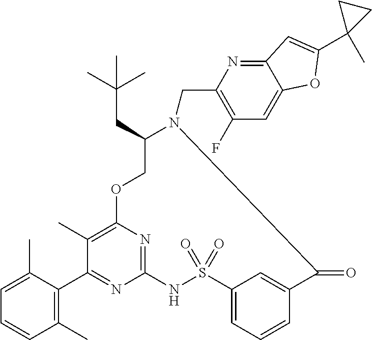 [SMILES: Cc1cccc(C)c1-c1nc2nc(c1C)OC[C@@H](CC(C)(C)C)N(Cc1nc3cc(C4(C)CC4)oc3cc1F)C(=O)c1cccc(c1)S(=O)(=O)N2] | | | | +++ |
| I-237 | 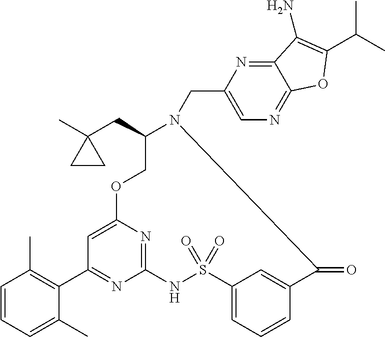 [SMILES: Cc1cccc(C)c1-c1cc2nc(n1)NS(=O)(=O)c1cccc(c1)C(=O)N(Cc1cnc3oc(C(C)C)c(N)c3n1)[C@H](CC1(C)CC1)CO2] | | | | +++ |
| I-238 | 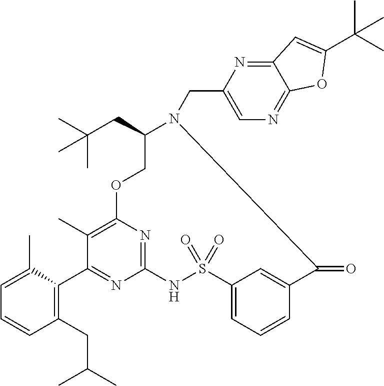 | | | | +++ |
| I-239 | 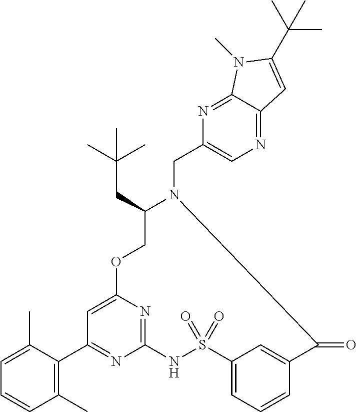 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](CC(C)(C)C)CO2] | | | +++ | +++ |
| I-240 | 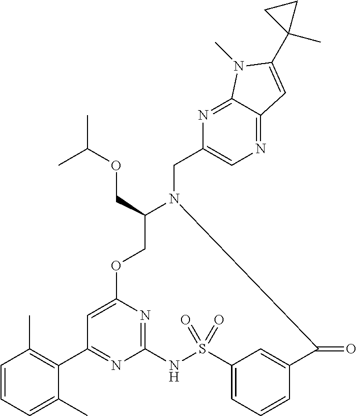 [SMILES: Cc1cccc(C)c1-c1cc2nc(n1)NS(=O)(=O)c1cccc(c1)C(=O)N(Cc1cnc3cc(C4(C)CC4)n(C)c3n1)[C@H](COC(C)C)CO2] | | | +++ | +++ |
| I-241 | 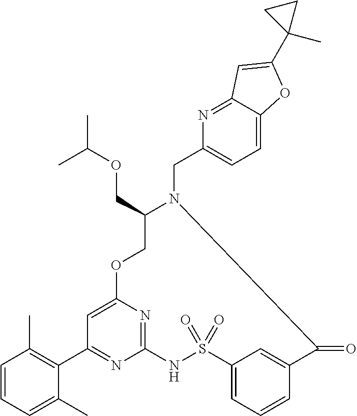 [SMILES: Cc1cccc(C)c1-c1cc2nc(n1)NS(=O)(=O)c1cccc(c1)C(=O)N(Cc1ccc3oc(C4(C)CC4)cc3n1)[C@H](COC(C)C)CO2] | | | | +++ |
| I-242 | 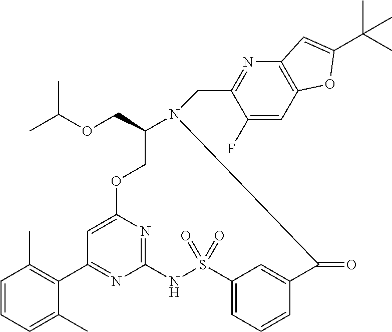 [SMILES: Cc1cccc(C)c1-c1cc2nc(n1)NS(=O)(=O)c1cccc(c1)C(=O)N(Cc1nc3cc(C(C)(C)C)oc3cc1F)[C@H](COC(C)C)CO2] | | | | +++ |
| I-243 | 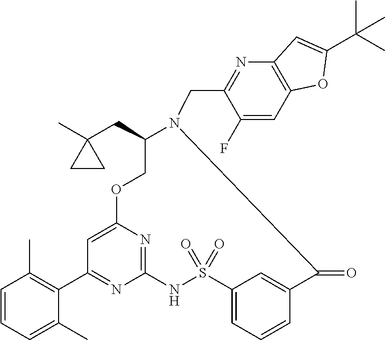 [SMILES: Cc1cccc(C)c1-c1cc2nc(n1)NS(=O)(=O)c1cccc(c1)C(=O)N(Cc1nc3cc(C(C)(C)C)oc3cc1F)[C@H](CC1(C)CC1)CO2] | | | | +++ |
| I-244 | 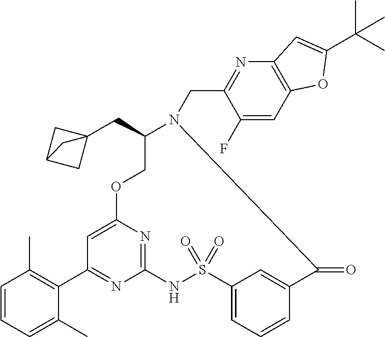 [SMILES: Cc1cccc(C)c1-c1cc2nc(n1)NS(=O)(=O)c1cccc(c1)C(=O)N(Cc1nc3cc(C(C)(C)C)oc3cc1F)[C@H](CC13CC(C1)C3)CO2] | | | | +++ |
| I-245 | 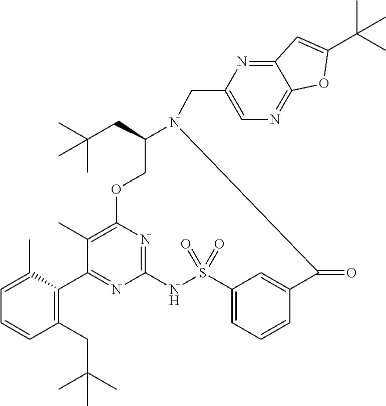 | | | +++ | |
| I-247 | 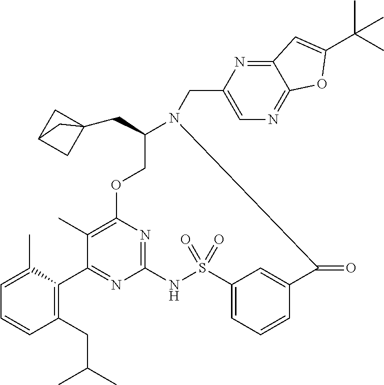 | | | +++ | |
| I-248 | 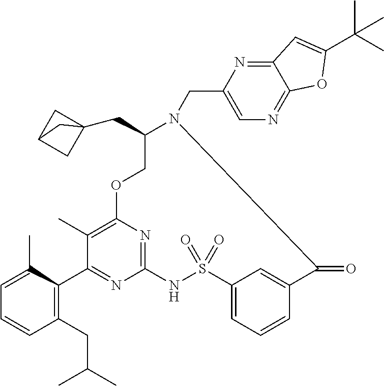 | | | | +++ |
| I-249 | 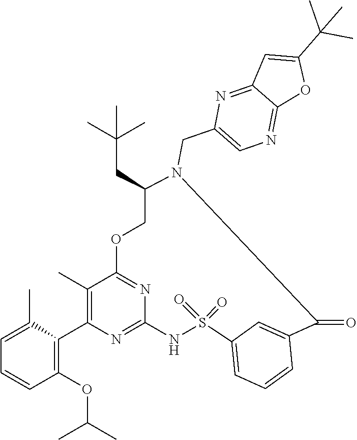 | | | +++ | |
| I-250 | 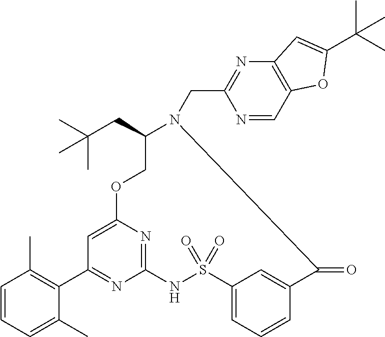 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC(C)(C)C)CO2] | | | | +++ |
| I-251 | 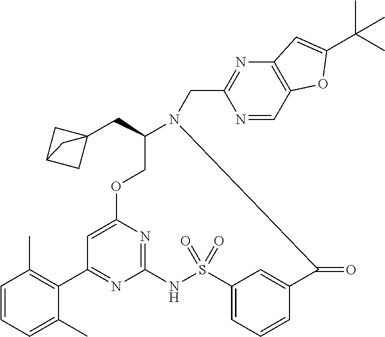 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC13CC(C1)C3)CO2] | | | +++ | |
| I-252 | 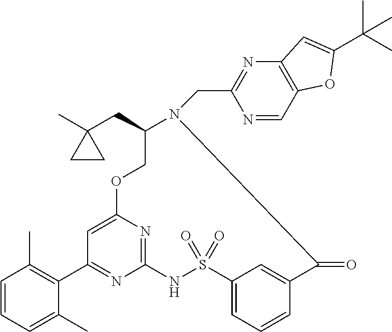 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC1(C)CC1)CO2] | | | | +++ |
| I-253 | 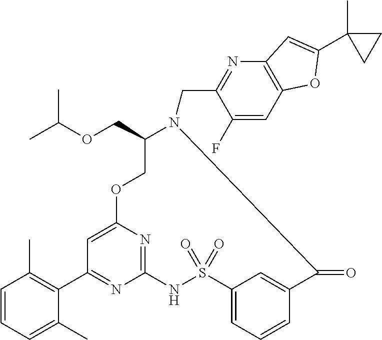 [SMILES: Cc1cccc(C)c1-c1cc2nc(n1)NS(=O)(=O)c1cccc(c1)C(=O)N(Cc1nc3cc(C4(C)CC4)oc3cc1F)[C@H](COC(C)C)CO2] | | | | +++ |
| I-254 | 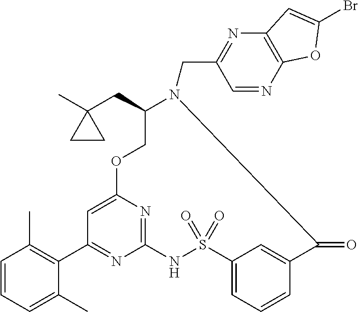 [SMILES: Cc1cccc(C)c1-c1cc2nc(n1)NS(=O)(=O)c1cccc(c1)C(=O)N(Cc1cnc3oc(Br)cc3n1)[C@H](CC1(C)CC1)CO2] | | | | ++ |
| I-255 | 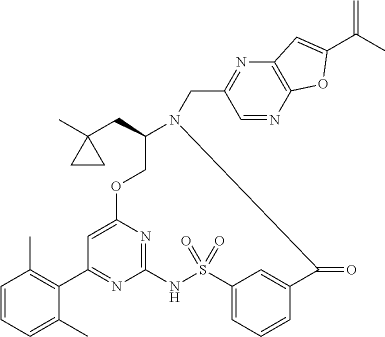 [SMILES: C=C(C)c1cc2nc(CN3C(=O)c4cccc(c4)S(=O)(=O)Nc4nc(cc(-c5c(C)cccc5C)n4)OC[C@H]3CC3(C)CC3)cnc2o1] | | | +++ | |
| I-256 | 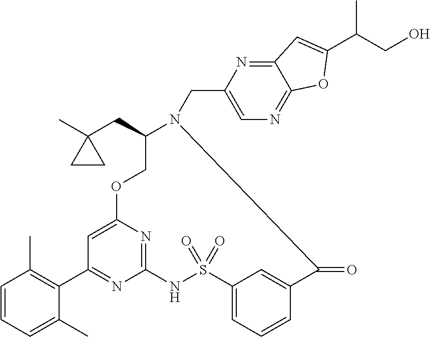 [SMILES: Cc1cccc(C)c1-c1cc2nc(n1)NS(=O)(=O)c1cccc(c1)C(=O)N(Cc1cnc3oc(C(C)CO)cc3n1)[C@H](CC1(C)CC1)CO2] | | | +++ | |
| I-257 | 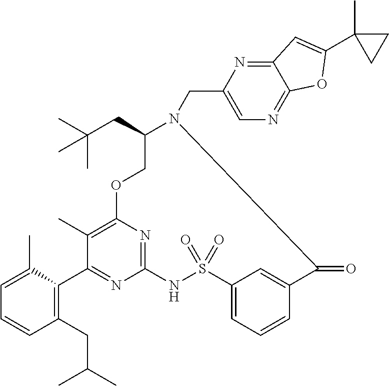 | | | +++ | |
| I-258 | 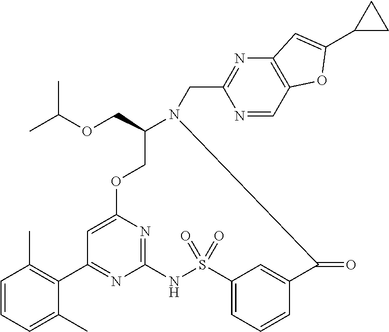 [SMILES: Cc1cccc(C)c1-c1cc2nc(n1)NS(=O)(=O)c1cccc(c1)C(=O)N(Cc1ncc3oc(C4CC4)cc3n1)[C@H](COC(C)C)CO2] | | | | +++ |
| I-259 | 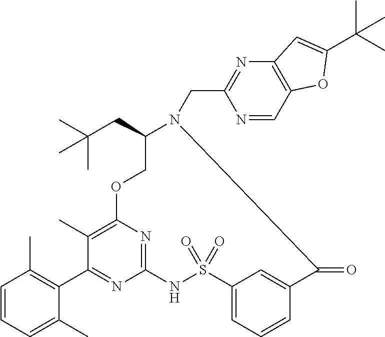 [SMILES: Cc1cccc(C)c1-c1nc2nc(c1C)OC[C@@H](CC(C)(C)C)N(Cc1ncc3oc(C(C)(C)C)cc3n1)C(=O)c1cccc(c1)S(=O)(=O)N2] | | | +++ | |
| I-260 | 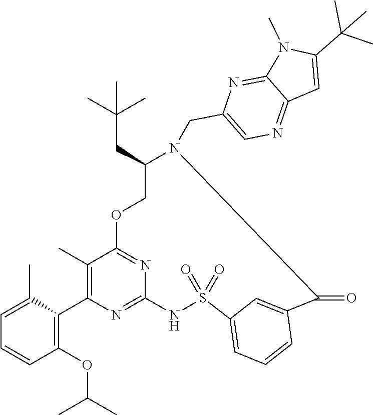 | | | +++ | |
| I-261 | 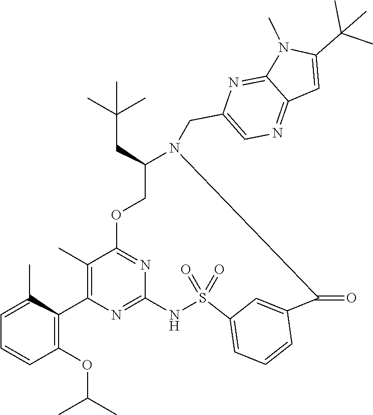 | | | | +++ |
| I-262 | 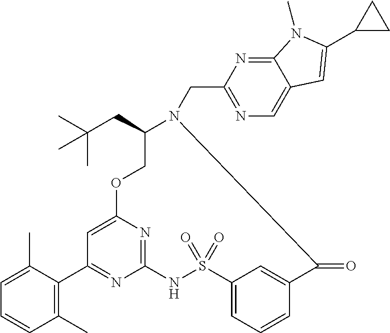 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC(C)(C)C)CO2] | | | +++ | +++ |
| I-263 | 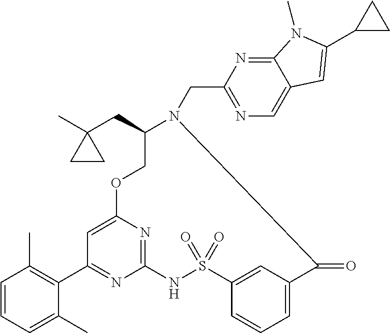 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC1(C)CC1)CO2] | | | +++ | +++ |
| I-264 | 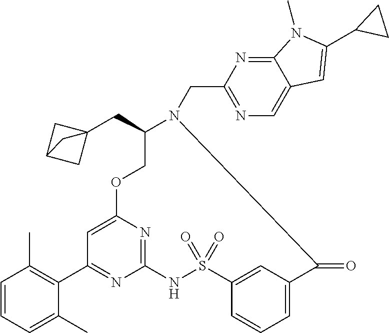 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC13CC(C1)C3)CO2] | | | +++ | +++ |
| I-265 | 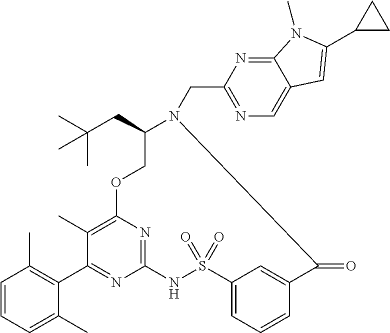 [SMILES: Cc1cccc(C)c1-c1nc2nc(c1C)OC[C@@H](CC(C)(C)C)N(Cc1ncc3cc(C4CC4)n(C)c3n1)C(=O)c1cccc(c1)S(=O)(=O)N2] | | | +++ | +++ |
| II-32 | 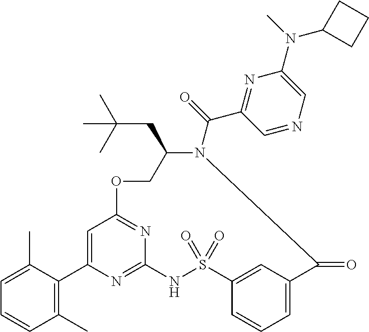 [SMILES: Cc1cccc(C)c1-c1cc2nc(n1)NS(=O)(=O)c1cccc(c1)C(=O)N(C(=O)c1cncc(N(C)C3CCC3)n1)[C@H](CC(C)(C)C)CO2] | +++ | | | |
| II-36 | 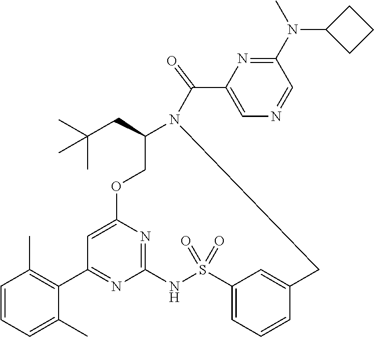 [SMILES: Cc1cccc(C)c1-c1cc2nc(n1)NS(=O)(=O)c1cccc(c1)CN(C(=O)c1cncc(N(C)C3CCC3)n1)[C@H](CC(C)(C)C)CO2] | ++ | | | |
| III-1 | 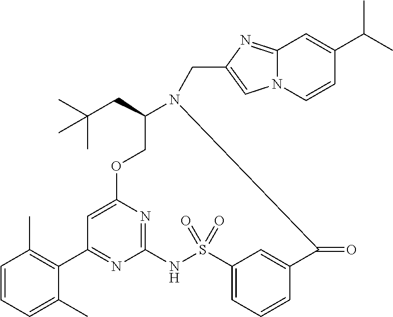 [SMILES: Cc1cccc(C)c1-c1cc2nc(n1)NS(=O)(=O)c1cccc(c1)C(=O)N(Cc1cn3ccc(C(C)C)cc3n1)[C@H](CC(C)(C)C)CO2] | +++ | | | |
| III-5 | 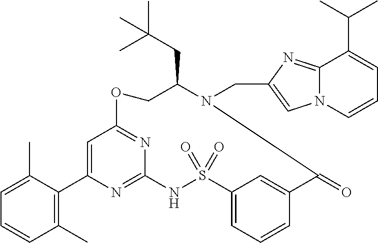 [SMILES: Cc1cccc(C)c1-c1cc2nc(n1)NS(=O)(=O)c1cccc(c1)C(=O)N(Cc1cn3cccc(C(C)C)c3n1)[C@H](CC(C)(C)C)CO2] | | | | +++ |
| III-7 | 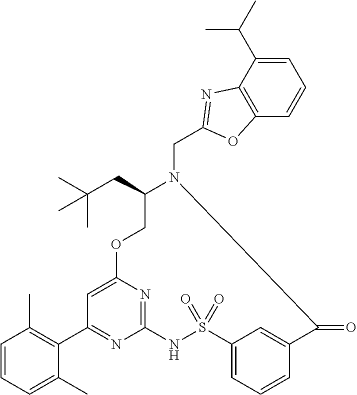 [SMILES: Cc1cccc(C)c1-c1cc2nc(n1)NS(=O)(=O)c1cccc(c1)C(=O)N(Cc1nc3c(C(C)C)cccc3o1)[C@H](CC(C)(C)C)CO2] | +++ | | | |
| III-10 | 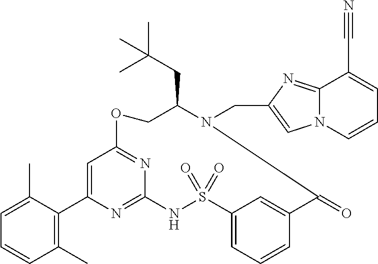 [SMILES: Cc1cccc(C)c1-c1cc2nc(n1)NS(=O)(=O)c1cccc(c1)C(=O)N(Cc1cn3cccc(C#N)c3n1)[C@H](CC(C)(C)C)CO2] | +++ | | | |
| III-12 | 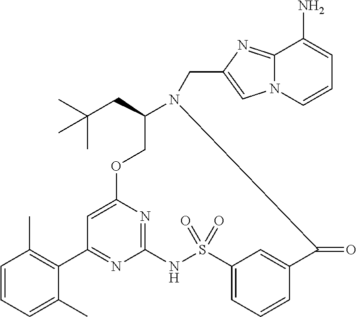 [SMILES: Cc1cccc(C)c1-c1cc2nc(n1)NS(=O)(=O)c1cccc(c1)C(=O)N(Cc1cn3cccc(N)c3n1)[C@H](CC(C)(C)C)CO2] | +++ | | | |
| III-13 | 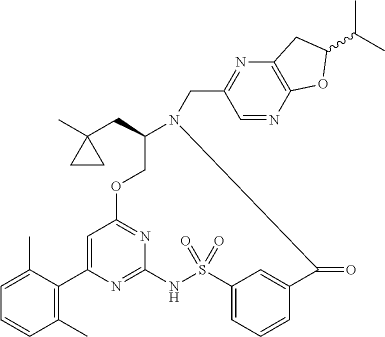 [SMILES: Cc1cccc(C)c1-c1cc2nc(n1)NS(=O)(=O)c1cccc(c1)C(=O)N(Cc1cnc3c(n1)CC(C(C)C)O3)[C@H](CC1(C)CC1)CO2] | +++ | | | |
| III-14 | 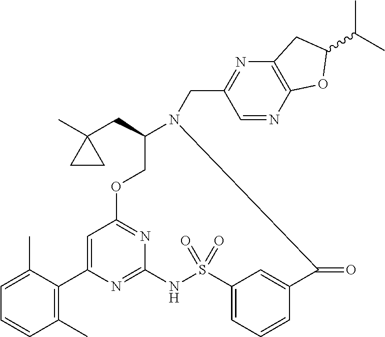 [SMILES: Cc1cccc(C)c1-c1cc2nc(n1)NS(=O)(=O)c1cccc(c1)C(=O)N(Cc1cnc3c(n1)CC(C(C)C)O3)[C@H](CC1(C)CC1)CO2] | +++ | | | |
| III-15 | 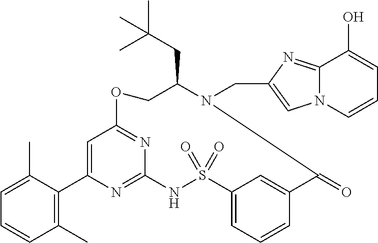 [SMILES: Cc1cccc(C)c1-c1cc2nc(n1)NS(=O)(=O)c1cccc(c1)C(=O)N(Cc1cn3cccc(O)c3n1)[C@H](CC(C)(C)C)CO2] | | | | +++ |
| III-16 | 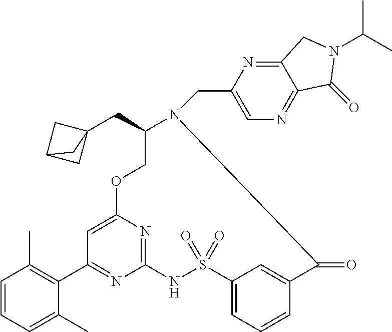 [SMILES: Cc1cccc(C)c1-c1cc2nc(n1)NS(=O)(=O)c1cccc(c1)C(=O)N(Cc1cnc3c(n1)CN(C(C)C)C3=O)[C@H](CC13CC(C1)C3)CO2] | | | | +++ |
| III-17 | 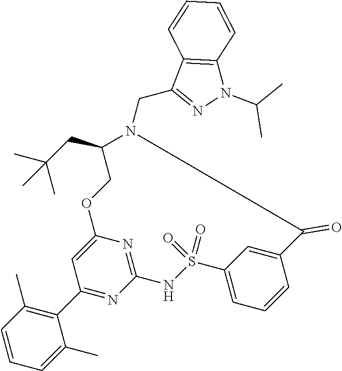 [SMILES: Cc1cccc(C)c1-c1cc2nc(n1)NS(=O)(=O)c1cccc(c1)C(=O)N(Cc1nn(C(C)C)c3ccccc13)[C@H](CC(C)(C)C)CO2] | | | | +++ |
| III-18 | 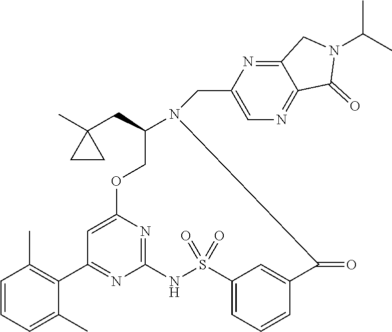 [SMILES: Cc1cccc(C)c1-c1cc2nc(n1)NS(=O)(=O)c1cccc(c1)C(=O)N(Cc1cnc3c(n1)CN(C(C)C)C3=O)[C@H](CC1(C)CC1)CO2] | | | +++ | |
| III-19 | 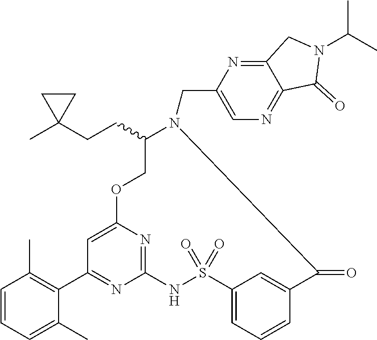 [SMILES: Cc1cccc(C)c1-c1cc2nc(n1)NS(=O)(=O)c1cccc(c1)C(=O)N(Cc1cnc3c(n1)CN(C(C)C)C3=O)C(CCC1(C)CC1)CO2] | | | | +++ |
| III-20 | 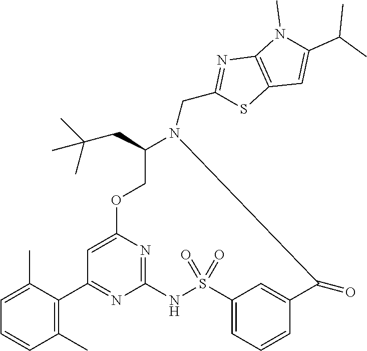 [SMILES: Cc1cccc(C)c1-c1cc2nc(n1)NS(=O)(=O)c1cccc(c1)C(=O)N(Cc1nc3c(cc(C(C)C)n3C)s1)[C@H](CC(C)(C)C)CO2] | | | | +++ |
| III-21 | 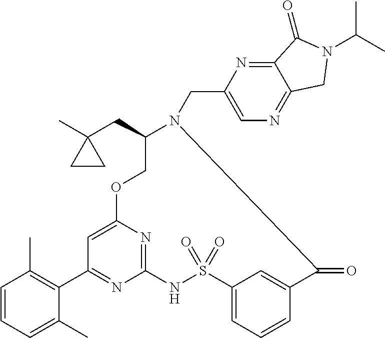 [SMILES: Cc1cccc(C)c1-c1cc2nc(n1)NS(=O)(=O)c1cccc(c1)C(=O)N(Cc1cnc3c(n1)C(=O)N(C(C)C)C3)[C@H](CC1(C)CC1)CO2] | | | +++ | |
| III-22 | 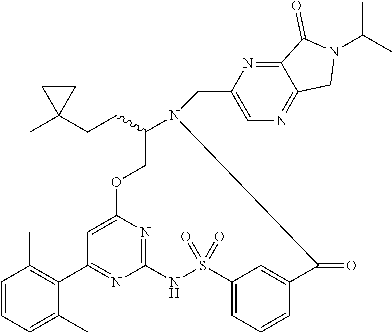 [SMILES: Cc1cccc(C)c1-c1cc2nc(n1)NS(=O)(=O)c1cccc(c1)C(=O)N(Cc1cnc3c(n1)C(=O)N(C(C)C)C3)C(CCC1(C)CC1)CO2] | | | | +++ |
| III-23 | 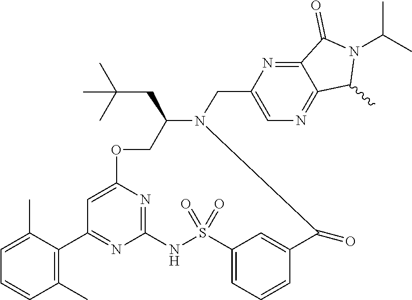 [SMILES: Cc1cccc(C)c1-c1cc2nc(n1)NS(=O)(=O)c1cccc(c1)C(=O)N(Cc1cnc3c(n1)C(=O)N(C(C)C)C3C)[C@H](CC(C)(C)C)CO2] | | | | +++ |
| III-24 | 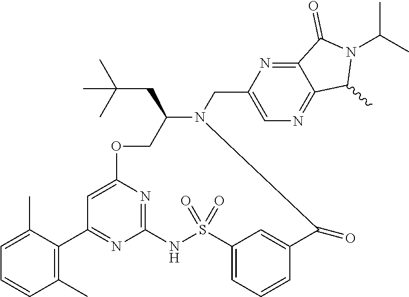 [SMILES: Cc1cccc(C)c1-c1cc2nc(n1)NS(=O)(=O)c1cccc(c1)C(=O)N(Cc1cnc3c(n1)C(=O)N(C(C)C)C3C)[C@H](CC(C)(C)C)CO2] | | | | +++ |
| III-25 | 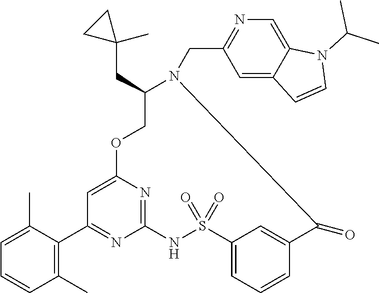 [SMILES: Cc1cccc(C)c1-c1cc2nc(n1)NS(=O)(=O)c1cccc(c1)C(=O)N(Cc1cc3ccn(C(C)C)c3cn1)[C@H](CC1(C)CC1)CO2] | | | | +++ |

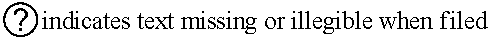

| Com- | | A | B | C |
|---|---|---|---|---|
| pound | | EC50 | EC50 | EC50 |
| Number | Structure | (μM) | (μM) | (μM) |
| I-1 | 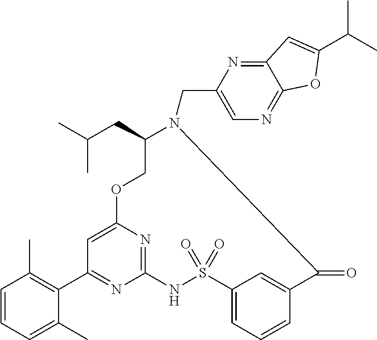 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)C)CO2] | +++ | | |
| I-2 | 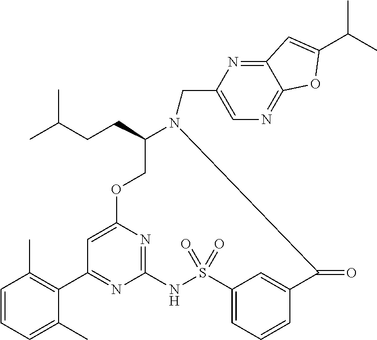 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)C)CO2] | +++ | | |
| I-3 | 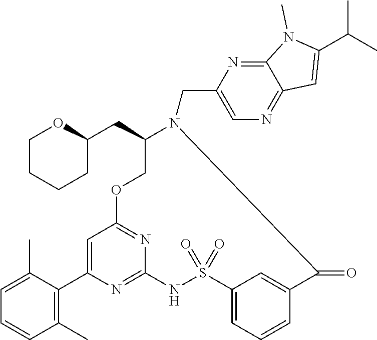 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCCO1)CO2] | +++ | | |
| I-4 | 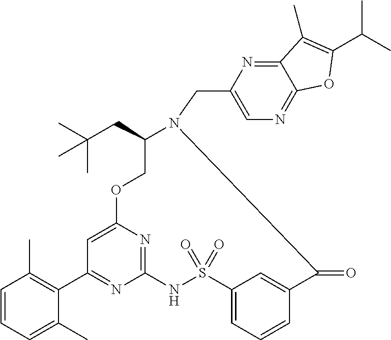 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)C)CO2] | +++ | | |
| I-6 | 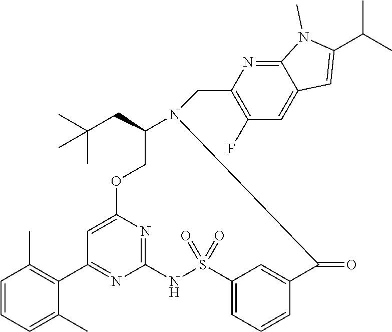 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC(C)(C)C)CO2] | +++ | | |
| I-7 | 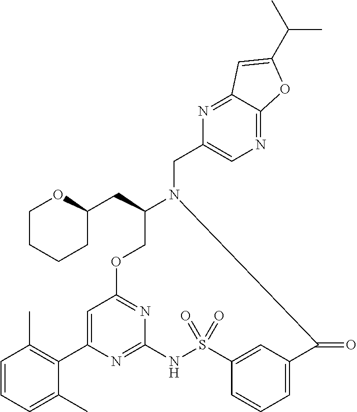 [SMILES: Cc1cccc(C)c1-c1cc2nc(n1)NS(=O)(=O)c1cccc(c1)C(=O)N(Cc1cnc3oc(C(C)C)cc3n1)[C@H](C[C@H]1CCCCO1)CO2] | +++ | | |
| I-9 | 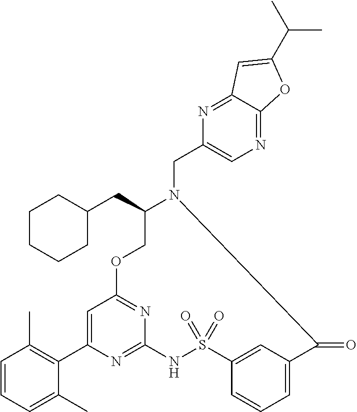 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCCC1)CO2] | +++ | | |
| I-10 | 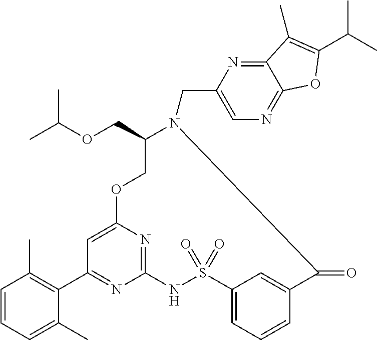 [SMILES: Cc1cccc(C)c1-c1cc2nc(n1)NS(=O)(=O)c1cccc(c1)C(=O)N(Cc1cnc3oc(C(C)C)c(C)c3n1)[C@H](COC(C)C)CO2] | +++ | | |
| I-11 | 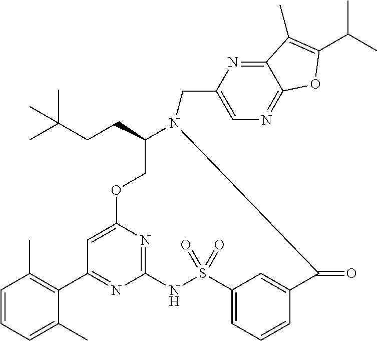 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)(C)C)CO2] | +++ | | |
| I-12 | 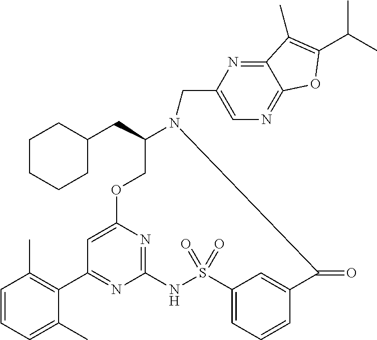 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCCC1)CO2] | +++ | | |
| I-13 | 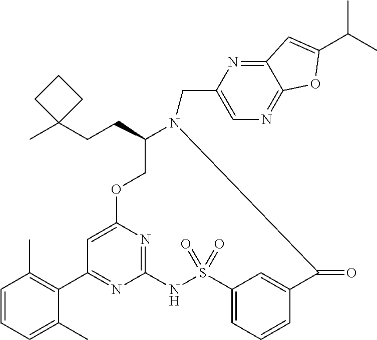 [SMILES: Cc1cccc(C)c1-c1cc2nc(n1)NS(=O)(=O)c1cccc(c1)C(=O)N(Cc1cnc3oc(C(C)C)cc3n1)[C@H](CCC1(C)CCC1)CO2] | +++ | | |
| I-16 | 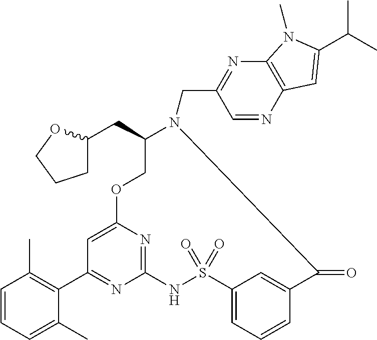 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCO1)CO2] | +++ | | |
| I-17 | 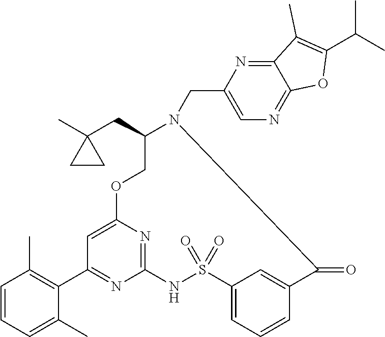 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1(C)CC1)CO2] | +++ | | |
| I-18 | 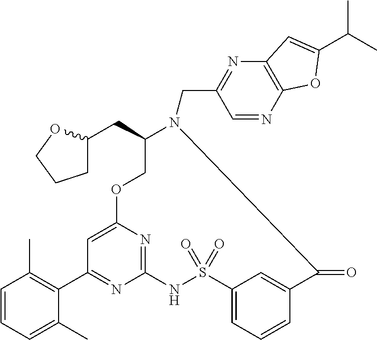 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCO1)CO2] | +++ | | |
| I-19 | 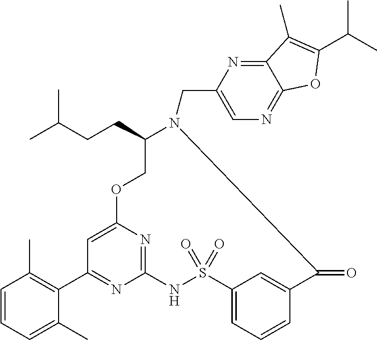 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC(C)C)CO2] | +++ | | |
| I-20 | 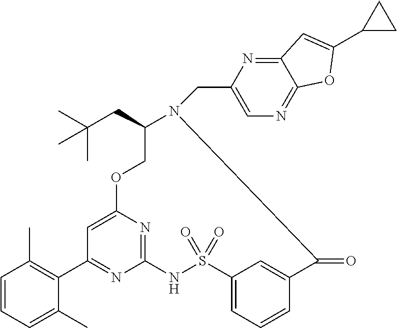 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)C)CO2] | +++ | | |
| I-21 | 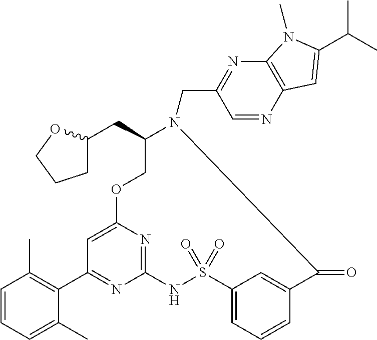 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCO1)CO2] | +++ | | |
| I-23 | 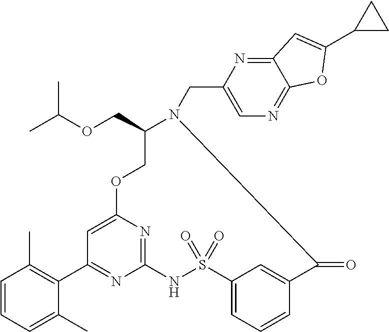 [SMILES: Cc1cccc(C)c1-c1cc2nc(n1)NS(=O)(=O)c1cccc(c1)C(=O)N(Cc1cnc3oc(C4CC4)cc3n1)[C@H](COC(C)C)CO2] | +++ | ++ | |
| I-24 | 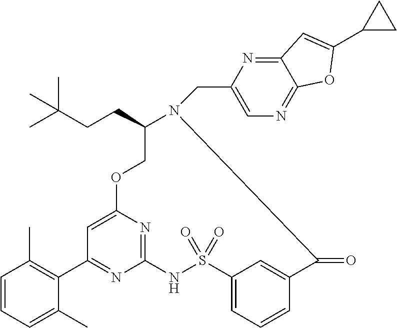 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)C)CO2] | +++ | ++ | |
| I-25 | 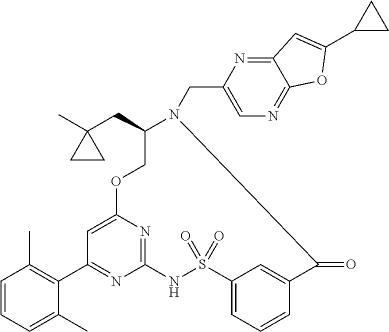 [SMILES: Cc1cccc(C)c1-c1cc2nc(n1)NS(=O)(=O)c1cccc(c1)C(=O)N(Cc1cnc3oc(C4CC4)cc3n1)[C@H](CC1(C)CC1)CO2] | +++ | | |
| I-26 | 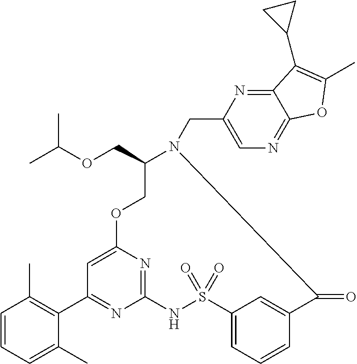 [SMILES: Cc1cccc(C)c1-c1cc2nc(n1)NS(=O)(=O)c1cccc(c1)C(=O)N(Cc1cnc3oc(C)c(C4CC4)c3n1)[C@H](COC(C)C)CO2] | +++ | | |
| I-27 | 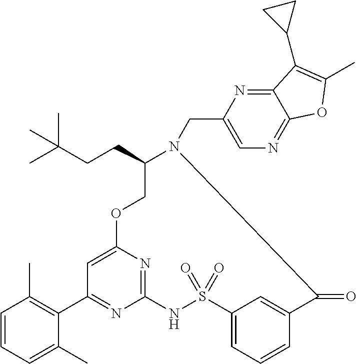 [SMILES: Cc1cccc(C)c1-c1cc2nc(n1)NS(=O)(=O)c1cccc(c1)C(=O)N(Cc1cnc3oc(C)c(C4CC4)c3n1)[C@H](CCC(C)(C)C)CO2] | +++ | | |
| I-30 | 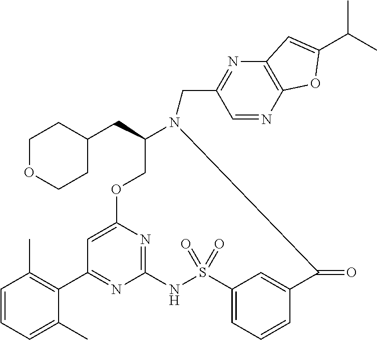 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCOCC1)CO2] | +++ | | |
| I-32 | 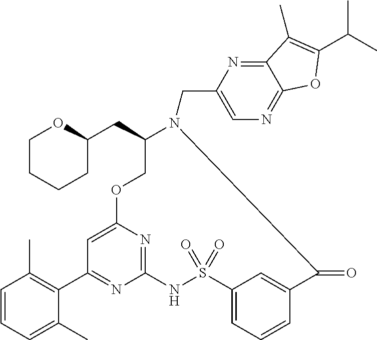 [SMILES: Cc1cccc(C)c1-c1cc2nc(n1)NS(=O)(=O)c1cccc(c1)C(=O)N(Cc1cnc3oc(C(C)C)c(C)c3n1)[C@H](C[C@H]1CCCCO1)CO2] | +++ | | |
| I-34 | 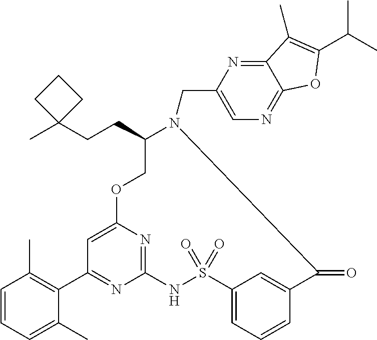 [SMILES: Cc1cccc(C)c1-c1cc2nc(n1)NS(=O)(=O)c1cccc(c1)C(=O)N(Cc1cnc3oc(C(C)C)c(C)c3n1)[C@H](CCC1(C)CCC1)CO2] | +++ | | |
| I-35 | 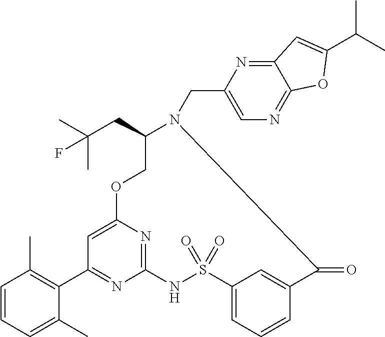 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)F)CO2] | +++ | | |
| I-36 | 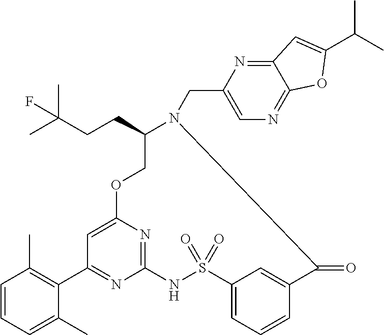 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)(C)F)CO2] | +++ | | |
| I-38 | 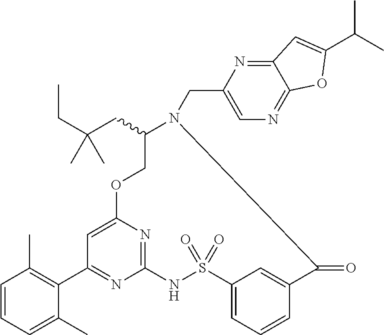 [SMILES: CCC(C)(C)CC1COc2cc(-c3c(C)cccc3C)nc(n2)NS(=O)(=O)c2cccc(c2)C(=O)N1Cc1cnc2oc(C(C)C)cc2n1] | | | +++ |
| I-40 | 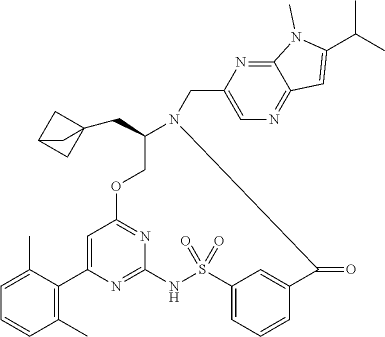 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC13CC(C1)C3)CO2] | +++ | | |
| I-41 | 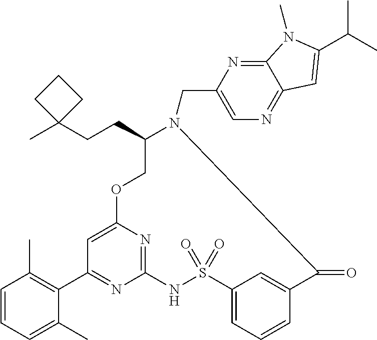 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1(C)CCC1)CO2] | +++ | | |
| I-42 | 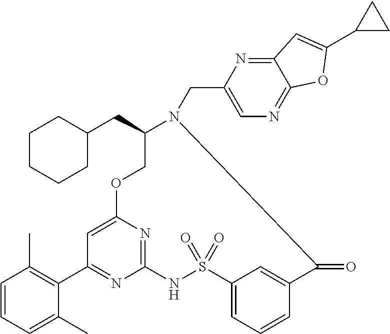 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCCC1)CO2] | +++ | | |
| I-43 | 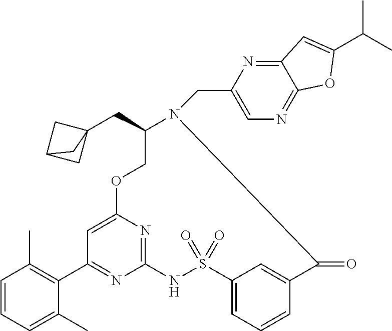 [SMILES: Cc1cccc(C)c1-c1cc2nc(n1)NS(=O)(=O)c1cccc(c1)C(=O)N(Cc1cnc3oc(C(C)C)cc3n1)[C@H](CC13CC(C1)C3)CO2] | +++ | + | |
| I-44 | 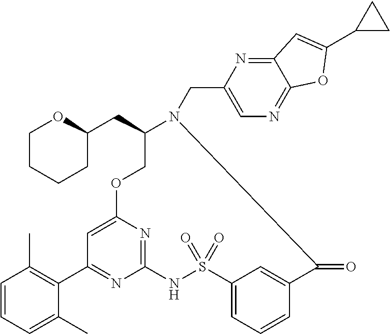 [SMILES: Cc1cccc(C)c1-c1cc2nc(n1)NS(=O)(=O)c1cccc(c1)C(=O)N(Cc1cnc3oc(C4CC4)cc3n1)[C@H](C[C@H]1CCCCO1)CO2] | +++ | | |
| I-47 | 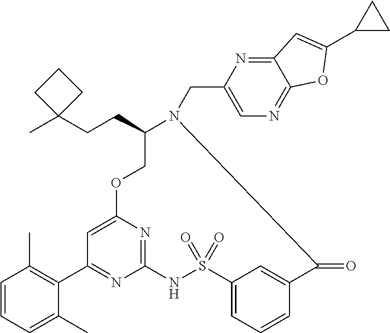 [SMILES: Cc1cccc(C)c1-c1cc2nc(n1)NS(=O)(=O)c1cccc(c1)C(=O)N(Cc1cnc3oc(C4CC4)cc3n1)[C@H](CCC1(C)CCC1)CO2] | +++ | | |
| I-49 | 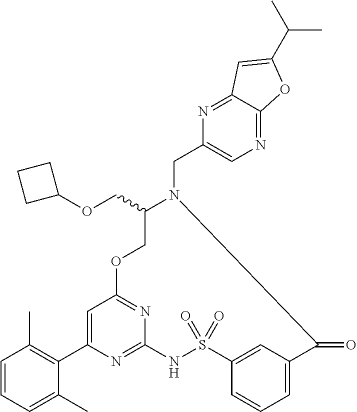 [SMILES: Cc1cccc(C)c1-c1cc2nc(n1)NS(=O)(=O)c1cccc(c1)C(=O)N(Cc1cnc3oc(C(C)C)cc3n1)C(COC1CCC1)CO2] | +++ | | |
| I-51 | 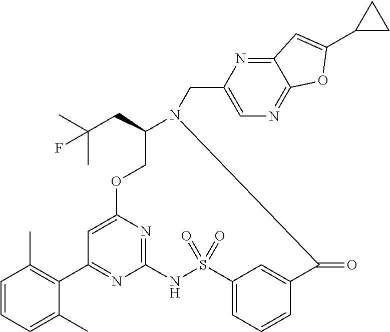 [SMILES: Cc1cccc(C)c1-c1cc2nc(n1)NS(=O)(=O)c1cccc(c1)C(=O)N(Cc1cnc3oc(C4CC4)cc3n1)[C@H](CC(C)(C)F)CO2] | +++ | | |
| I-52 | 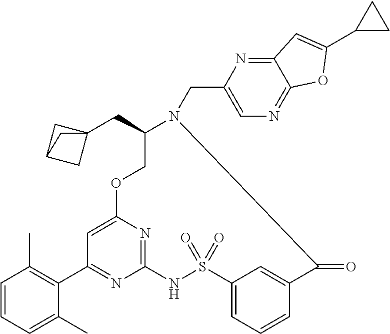 [SMILES: Cc1cccc(C)c1-c1cc2nc(n1)NS(=O)(=O)c1cccc(c1)C(=O)N(Cc1cnc3oc(C4CC4)cc3n1)[C@H](CC13CC(C1)C3)CO2] | +++ | ++ | |
| I-53 | 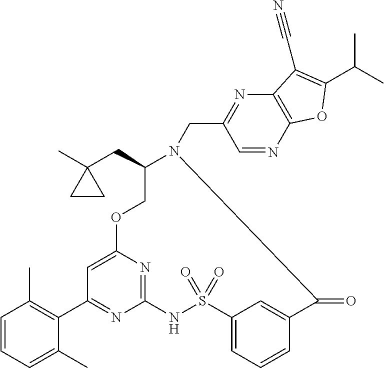 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC1(C)CC1)CO2] | +++ | | |
| I-54 | 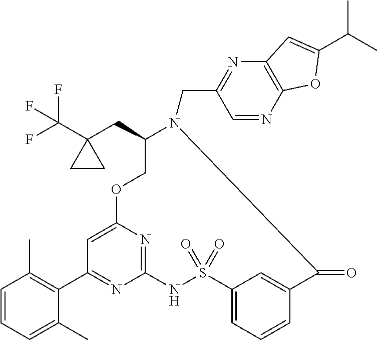 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C(F)(F)F)CC1)CO2] | +++ | | |
| I-56 | 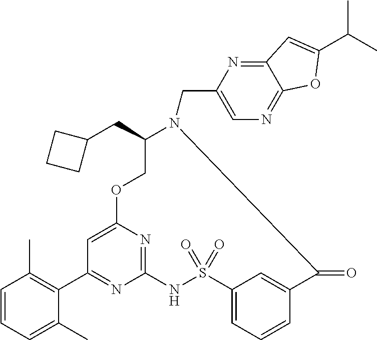 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCC1)CO2] | +++ | ++ | |
| I-57 | 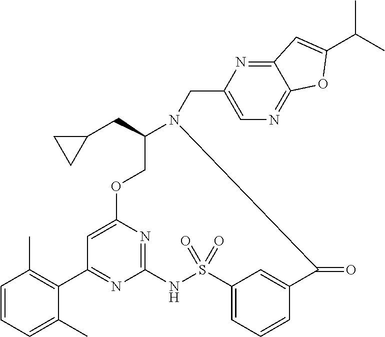 [SMILES: Cc1cccc(C)c1-c1cc2nc(n1)NS(=O)(=O)c1cccc(c1)C(=O)N(Cc1cnc3oc(C(C)C)cc3n1)[C@H](CC1CC1)CO2] | +++ | + | |
| I-58 | 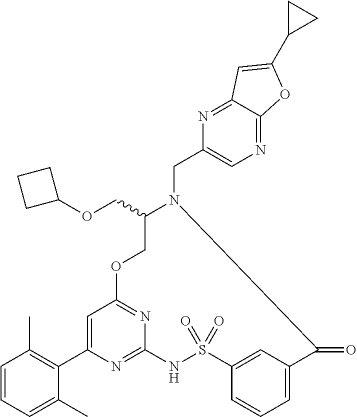 [SMILES: Cc1cccc(C)c1-c1cc2nc(n1)NS(=O)(=O)c1cccc(c1)C(=O)N(Cc1cnc3oc(C4CC4)cc3n1)C(COC1CCC1)CO2] | +++ | | |
| I-61 | 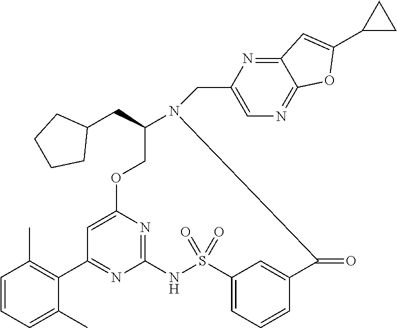 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCCC1)CO2] | | + | |
| I-62 | 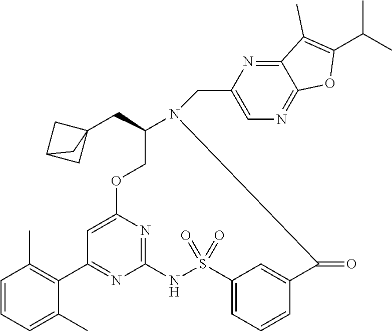 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC13CC(C1)C3)CO2] | +++ | | |
| I-63 | 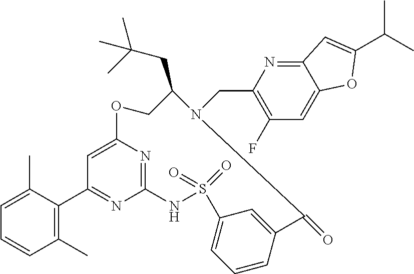 [SMILES: Cc1cccc(C)c1-c1cc2nc(n1)NS(=O)(=O)c1cccc(c1)C(=O)N(Cc1nc3cc(C(C)C)oc3cc1F)[C@H](CC(C)(C)C)CO2] | +++ | | |
| I-64 | 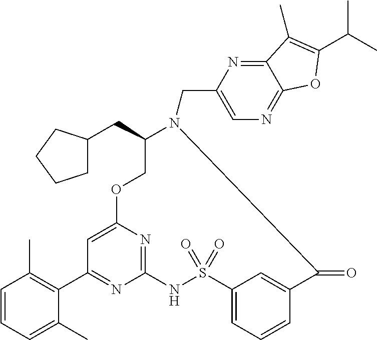 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC1CCCC1)CO2] | +++ | | |
| I-65 | 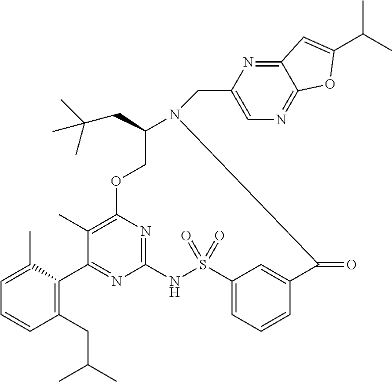 | +++ | ++ | |
| I-67 | 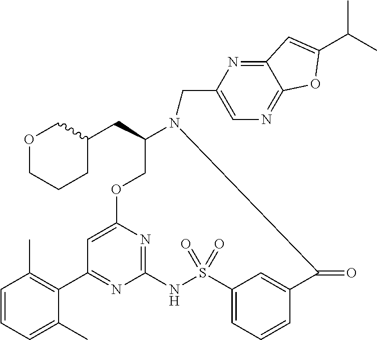 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCOC1)CO2] | +++ | | |
| I-69 | 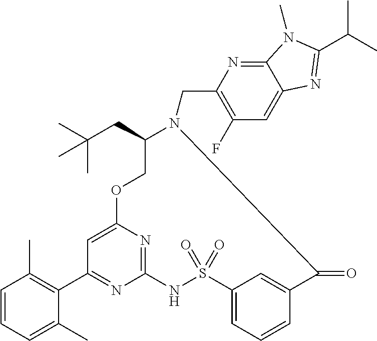 [SMILES: Cc1cccc(C)c1-c1cc2nc(n1)NS(=O)(=O)c1cccc(c1)C(=O)N(Cc1nc3c(cc1F)nc(C(C)C)n3C)[C@H](CC(C)(C)C)CO2] | +++ | | |
| I-70 | 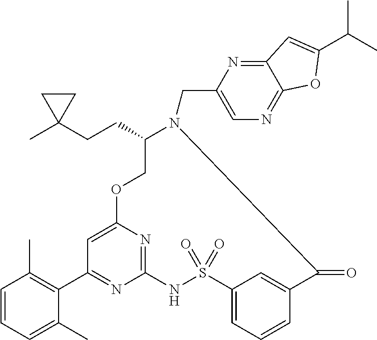 [SMILES: Cc1cccc(C)c1-c1cc2nc(n1)NS(=O)(=O)c1cccc(c1)C(=O)N(Cc1cnc3oc(C(C)C)cc3n1)[C@@H](CCC1(C)CC1)CO2] | +++ | + | |
| I-71 | 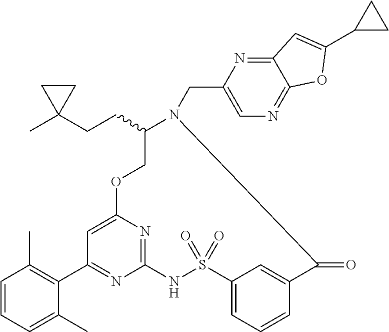 [SMILES: Cc1cccc(C)c1-c1cc2nc(n1)NS(=O)(=O)c1cccc(c1)C(=O)N(Cc1cnc3oc(C4CC4)cc3n1)C(CCC1(C)CC1)CO2] | +++ | + | |
| I-72 | 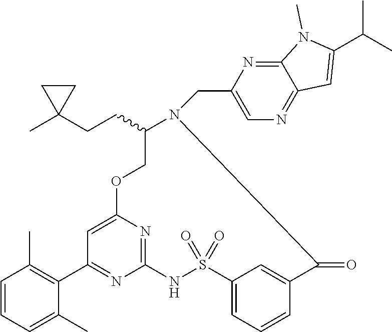 [SMILES: Cc1cccc(C)c1-c1cc2nc(n1)NS(=O)(=O)c1cccc(c1)C(=O)N(Cc1cnc3cc(C(C)C)n(C)c3n1)C(CCC1(C)CC1)CO2] | +++ | | |
| I-75 | 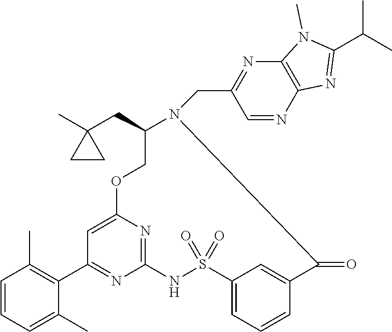 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC1(C)CC1)CO2] | +++ | | |
| I-78 | 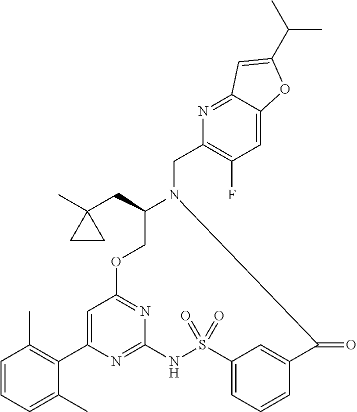 [SMILES: Cc1cccc(C)c1-c1cc2nc(n1)NS(=O)(=O)c1cccc(c1)C(=O)N(Cc1nc3cc(C(C)C)oc3cc1F)[C@H](CC1(C)CC1)CO2] | +++ | | |
| I-80 | 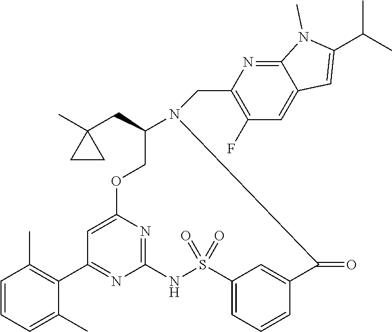 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC1(C)CC1)CO2] | +++ | | |
| I-82 | 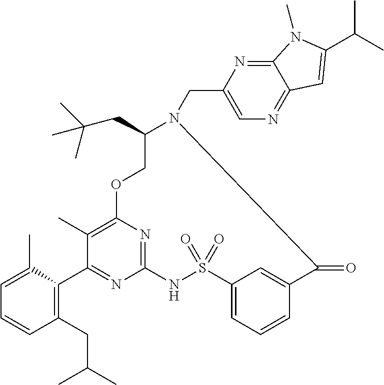 | +++ | +++ | |
| I-84 | 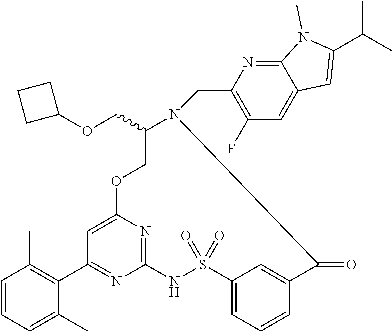 [SMILES: Cc1cccc(C)c1-c1cc2nc(n1)NS(=O)(=O)c1cccc(c1)C(=O)N(Cc1nc3c(cc1F)cc(C(C)C)n3C)C(COC1CCC1)CO2] | +++ | | |
| I-85 | 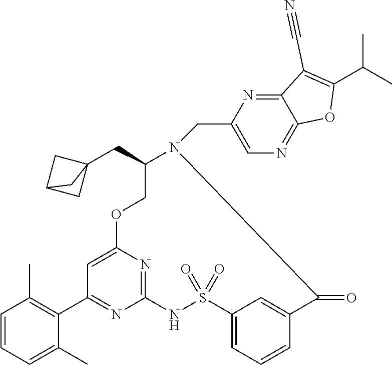 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](CC13CC(C1)C3)CO2] | +++ | | |
| I-86 | 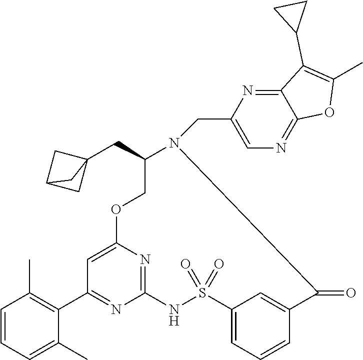 [SMILES: Cc1cccc(C)c1-c1cc2nc(n1)NS(=O)(=O)c1cccc(c1)C(=O)N(Cc1cnc3oc(C)c(C4CC4)c3n1)[C@H](CC13CC(C1)C3)CO2] | | | ++ |
| I-87 | 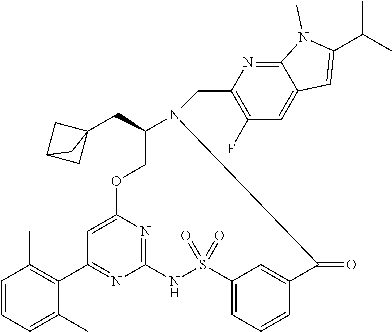 [SMILES: Cc1cccc(C)c1-c1cc2nc(n1)NS(=O)(=O)c1cccc(c1)C(=O)N(Cc1nc3c(cc1F)cc(C(C)C)n3C)[C@H](CC13CC(C1)C3)CO2] | +++ | | |
| I-88 | 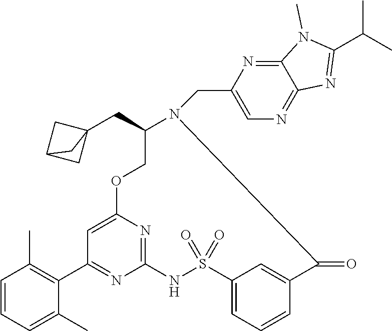 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC13CC(C1)C3)CO2] | +++ | +++ | |
| I-89 | 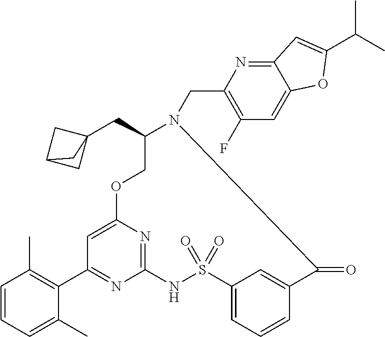 [SMILES: Cc1cccc(C)c1-c1cc2nc(n1)NS(=O)(=O)c1cccc(c1)C(=O)N(Cc1nc3cc(C(C)C)oc3cc1F)[C@H](CC13CC(C1)C3)CO2] | +++ | ++ | |
| I-90 | 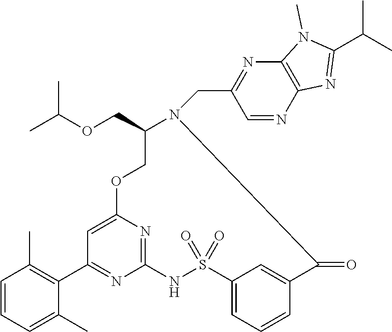 [SMILES: Cc1cccc(C)c1-c1cc2nc(n1)NS(=O)(=O)c1cccc(c1)C(=O)N(Cc1cnc3nc(C(C)C)n(C)c3n1)[C@H](COC(C)C)CO2] | +++ | | |
| I-91 | 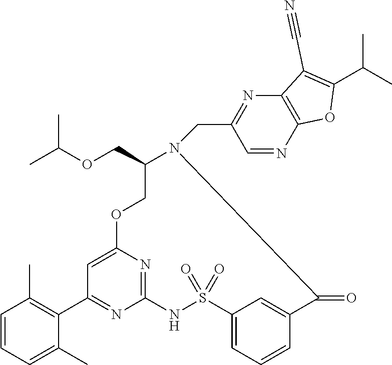 [SMILES: Cc1cccc(C)c1-c1cc2nc(n1)NS(=O)(=O)c1cccc(c1)C(=O)N(Cc1cnc3oc(C(C)C)c(C#N)c3n1)[C@H](COC(C)C)CO2] | +++ | | |
| I-93 | 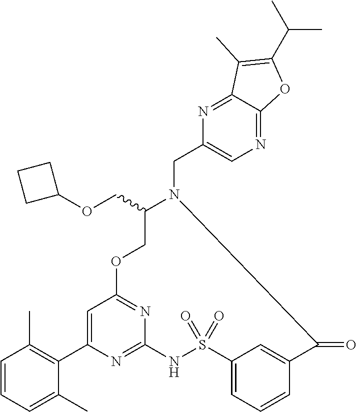 [SMILES: Cc1cccc(C)c1-c1cc2nc(n1)NS(=O)(=O)c1cccc(c1)C(=O)N(Cc1cnc3oc(C(C)C)c(C)c3n1)C(COC1CCC1)CO2] | +++ | | |
| I-95 | 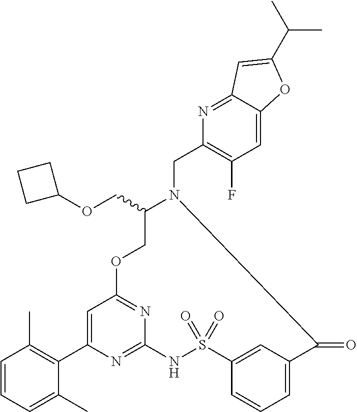 [SMILES: Cc1cccc(C)c1-c1cc2nc(n1)NS(=O)(=O)c1cccc(c1)C(=O)N(Cc1nc3cc(C(C)C)oc3cc1F)C(COC1CCC1)CO2] | +++ | | |
| I-96 | 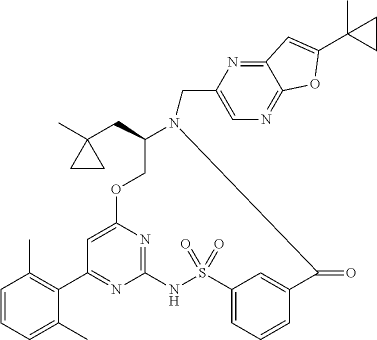 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC1(C)CC1)CO2] | +++ | ++ | |
| I-97 | 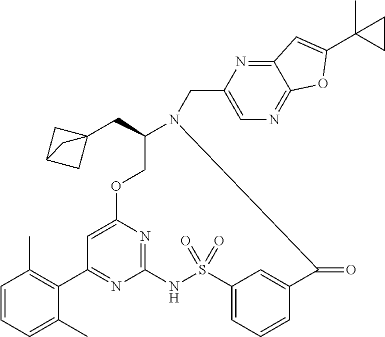 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC13CC(C1)C3)CO2] | +++ | ++ | |
| I-98 | 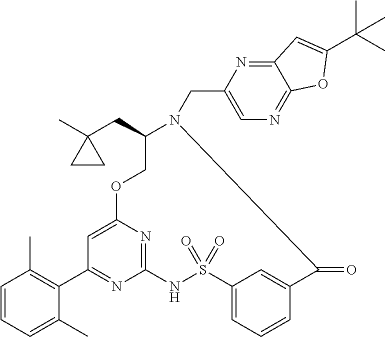 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1(C)CC1)CO2] | +++ | ++ | |
| I-99 | 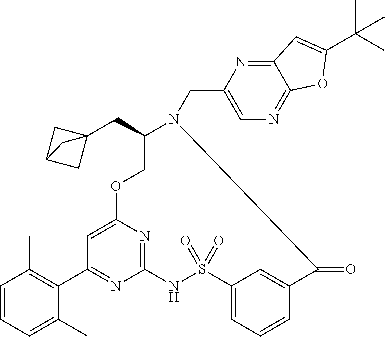 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC13CC(C1)C3)CO2] | +++ | +++ | |
| I-100 | 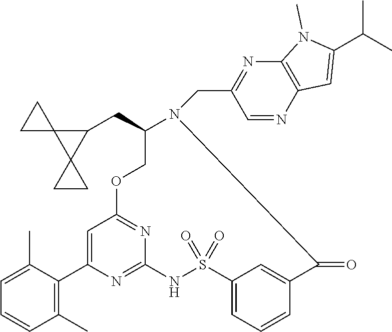 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1C3(CC3)C13CC3)CO2] | +++ | | |
| I-101 | 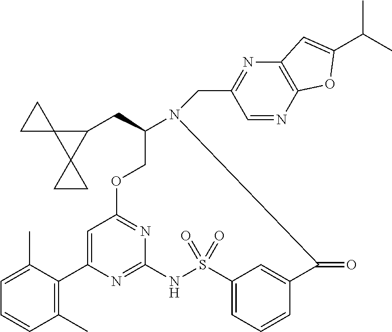 [SMILES: Cc1cccc(C)c1-c1cc2nc(n1)NS(=O)(=O)c1cccc(c1)C(=O)N(Cc1cnc3oc(C(C)C)cc3n1)[C@H](CC1C3(CC3)C13CC3)CO2] | +++ | | |
| I-102 | 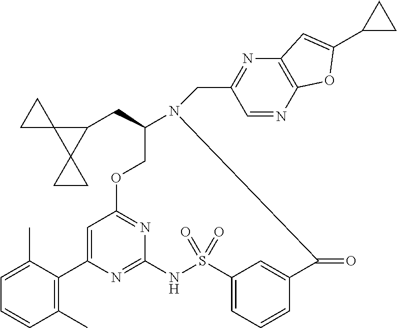 [SMILES: Cc1cccc(C)c1-c1cc2nc(n1)NS(=O)(=O)c1cccc(c1)C(=O)N(Cc1cnc3oc(C4CC4)cc3n1)[C@H](CC1C3(CC3)C13CC3)CO2] | +++ | | |
| I-103 | 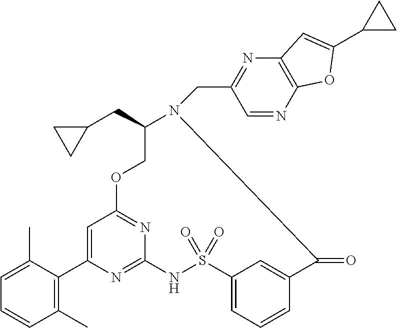 [SMILES: Cc1cccc(C)c1-c1cc2nc(n1)NS(=O)(=O)c1cccc(c1)C(=O)N(Cc1cnc3oc(C4CC4)cc3n1)[C@H](CC1CC1)CO2] | +++ | ++ | |
| I-104 | 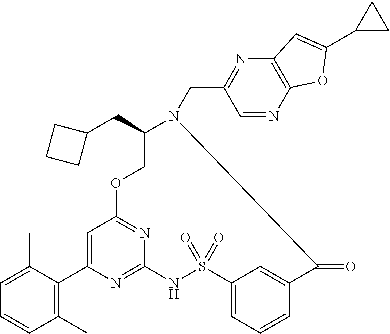 [SMILES: Cc1cccc(C)c1-c1cc2nc(n1)NS(=O)(=O)c1cccc(c1)C(=O)N(Cc1cnc3oc(C4CC4)cc3n1)[C@H](CC1CCC1)CO2] | +++ | + | |
| I-106 | 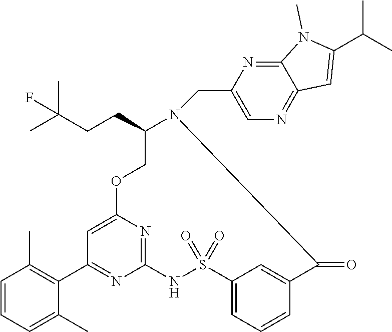 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)F)CO2] | +++ | | |
| I-107 | 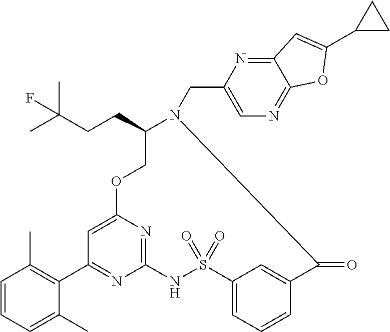 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)(C)F)CO2] | +++ | | |
| I-110 | 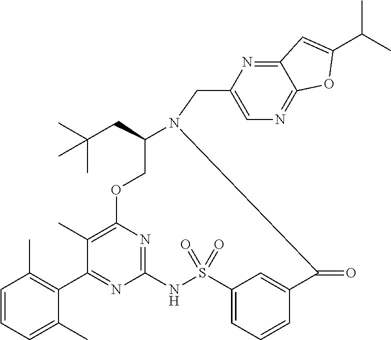 [SMILES: Cc1cccc(C)c1-c1nc2nc(c1C)OC[C@@H](CC(C)(C)C)N(Cc1cnc3oc(C(C)C)cc3n1)C(=O)c1cccc(c1)S(=O)(=O)N2] | | | ++ |
| I-112 | 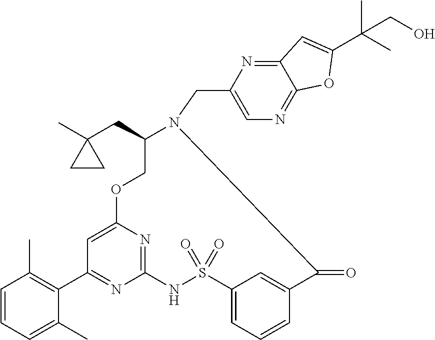 [SMILES: Cc1cccc(C)c1-c1cc2nc(n1)NS(=O)(=O)c1cccc(c1)C(=O)N(Cc1cnc3oc(C(C)(C)CO)cc3n1)[C@H](CC1(C)CC1)CO2] | | | + |
| I-113 | 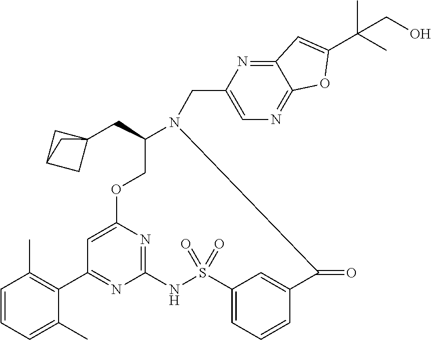 [SMILES: Cc1cccc(C)c1-c1cc2nc(n1)NS(=O)(=O)c1cccc(c1)C(=O)N(Cc1cnc3oc(C(C)(C)CO)cc3n1)[C@H](CC13CC(C1)C3)CO2] | | | + |
| I-118 | 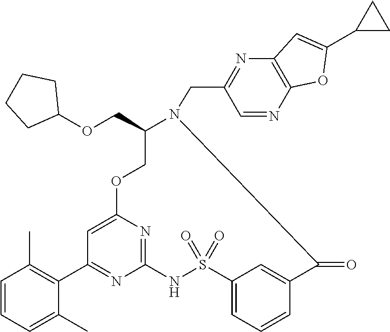 [SMILES: Cc1cccc(C)c1-c1cc2nc(n1)NS(=O)(=O)c1cccc(c1)C(=O)N(Cc1cnc3oc(C4CC4)cc3n1)[C@H](COC1CCCC1)CO2] | | + | |
| I-123 | 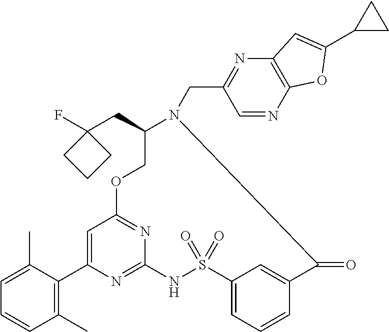 [SMILES: Cc1cccc(C)c1-c1cc2nc(n1)NS(=O)(=O)c1cccc(c1)C(=O)N(Cc1cnc3oc(C4CC4)cc3n1)[C@H](CC1(F)CCC1)CO2] | | + | |
| I-128 | 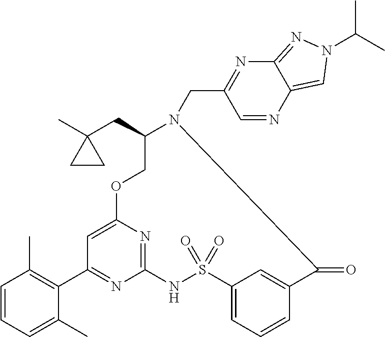 [SMILES: Cc1cccc(C)c1-c1cc2nc(n1)NS(=O)(=O)c1cccc(c1)C(=O)N(Cc1cnc3cn(C(C)C)nc3n1)[C@H](CC1(C)CC1)CO2] | | + | |
| I-130 | 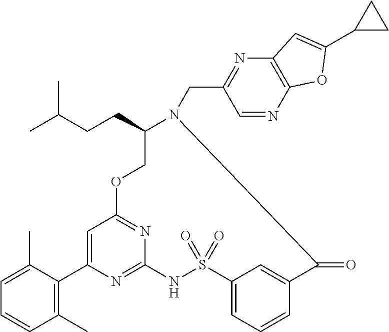 [SMILES: Cc1cccc(C)c1-c1cc2nc(n1)NS(=O)(=O)c1cccc(c1)C(=O)N(Cc1cnc3oc(C4CC4)cc3n1)[C@H](CCC(C)C)CO2] | | + | |
| I-133 | 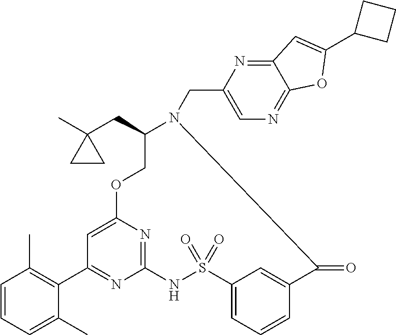 [SMILES: Cc1cccc(C)c1-c1cc2nc(n1)NS(=O)(=O)c1cccc(c1)C(=O)N(Cc1cnc3oc(C4CCC4)cc3n1)[C@H](CC1(C)CC1)CO2] | | | + |
| I-134 | 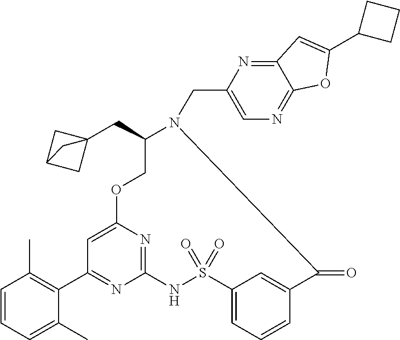 [SMILES: Cc1cccc(C)c1-c1cc2nc(n1)NS(=O)(=O)c1cccc(c1)C(=O)N(Cc1cnc3oc(C4CCC4)cc3n1)[C@H](CC13CC(C1)C3)CO2] | | +++ | |
| I-137 | 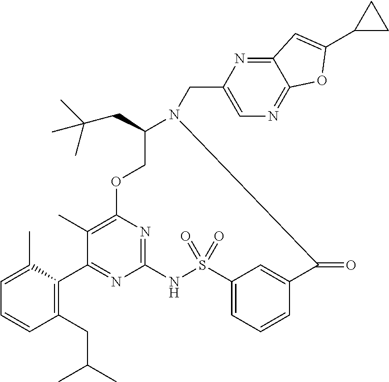 | | | +++ |
| I-138 | 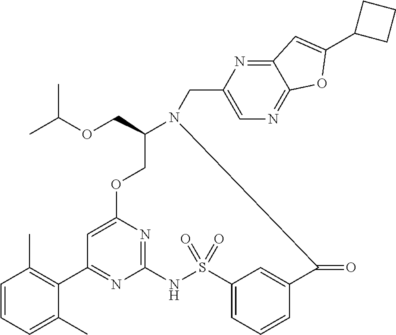 [SMILES: Cc1cccc(C)c1-c1cc2nc(n1)NS(=O)(=O)c1cccc(c1)C(=O)N(Cc1cnc3oc(C4CCC4)cc3n1)[C@H](COC(C)C)CO2] | | + | |
| I-139 | 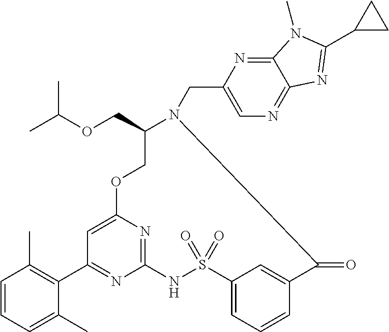 [SMILES: Cc1cccc(C)c1-c1cc2nc(n1)NS(=O)(=O)c1cccc(c1)C(=O)N(Cc1cnc3nc(C4CC4)n(C)c3n1)[C@H](COC(C)C)CO2] | | + | |
| I-143 | 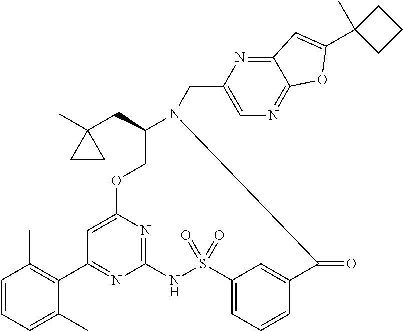 [SMILES: Cc1cccc(C)c1-c1cc2nc(n1)NS(=O)(=O)c1cccc(c1)C(=O)N(Cc1cnc3oc(C4(C)CCC4)cc3n1)[C@H](CC1(C)CC1)CO2] | | | + |
| I-145 | 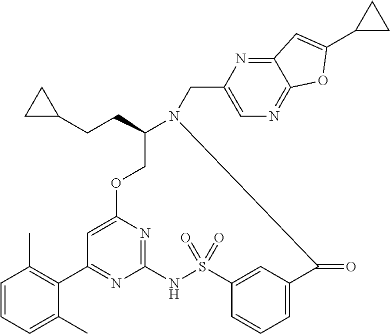 [SMILES: Cc1cccc(C)c1-c1cc2nc(n1)NS(=O)(=O)c1cccc(c1)C(=O)N(Cc1cnc3oc(C4CC4)cc3n1)[C@H](CCC1CC1)CO2] | | + | |
| I-151 | 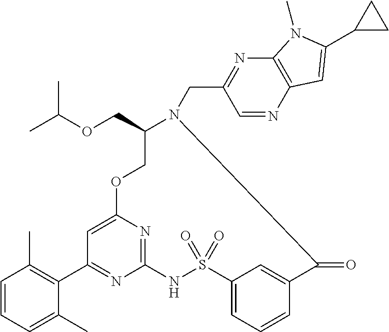 [SMILES: Cc1cccc(C)c1-c1cc2nc(n1)NS(=O)(=O)c1cccc(c1)C(=O)N(Cc1cnc3cc(C4CC4)n(C)c3n1)[C@H](COC(C)C)CO2] | | + | |
| I-152 | 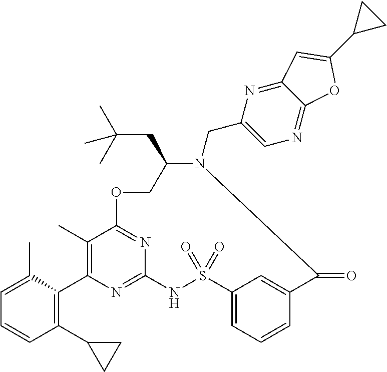 | | | +++ |
| I-158 | 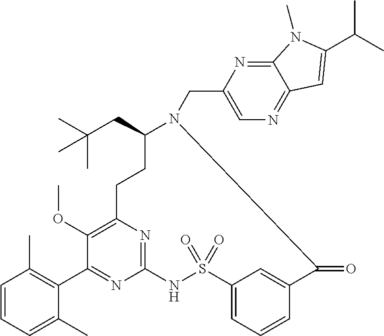 [SMILES: COc1c2nc(nc1-c1c(C)cccc1C)NS(=O)(=O)c1cccc(c1)C(=O)N(Cc1cnc3cc(C(C)C)n(C)c3n1)[C@H](CC(C)(C)C)CC2] | | | ++ |
| I-167 | 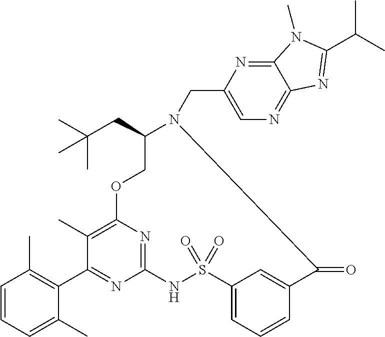 [SMILES: Cc1cccc(C)c1-c1nc2nc(c1C)OC[C@@H](CC(C)(C)C)N(Cc1cnc3nc(C(C)C)n(C)c3n1)C(=O)c1cccc(c1)S(=O)(=O)N2] | | +++ | |
| I-168 | 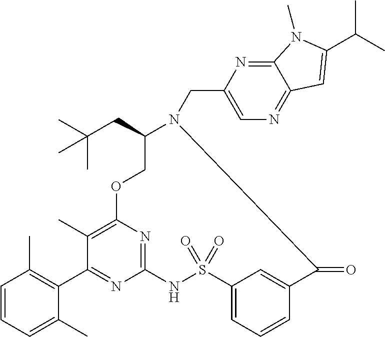 [SMILES: Cc1cccc(C)c1-c1nc2nc(c1C)OC[C@@H](CC(C)(C)C)N(Cc1cnc3cc(C(C)C)n(C)c3n1)C(=O)c1cccc(c1)S(=O)(=O)N2] | | | +++ |
| I-170 | 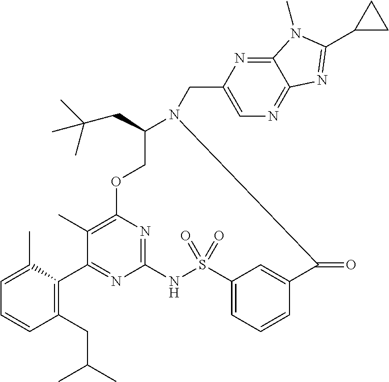 | | | +++ |
| I-171 | 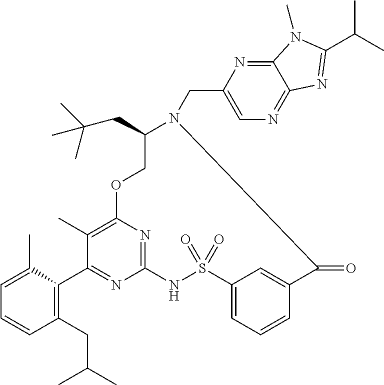 | | | +++ |
| I-174 | 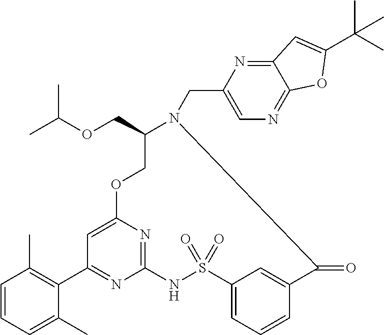 [SMILES: Cc1cccc(C)c1-c1cc2nc(n1)NS(=O)(=O)c1cccc(c1)C(=O)N(Cc1cnc3oc(C(C)(C)C)cc3n1)[C@H](COC(C)C)CO2] | | | + |
| I-177 | 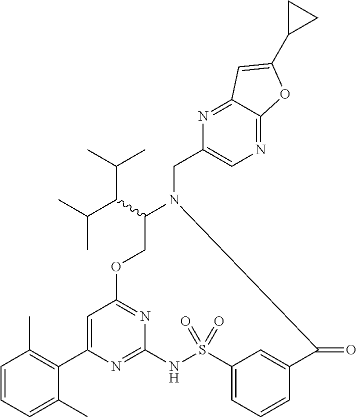 [SMILES: Cc1cccc(C)c1-c1cc2nc(n1)NS(=O)(=O)c1cccc(c1)C(=O)N(Cc1cnc3oc(C4CC4)cc3n1)C(C(C(C)C)C(C)C)CO2] | | | +++ |
| I-178 | 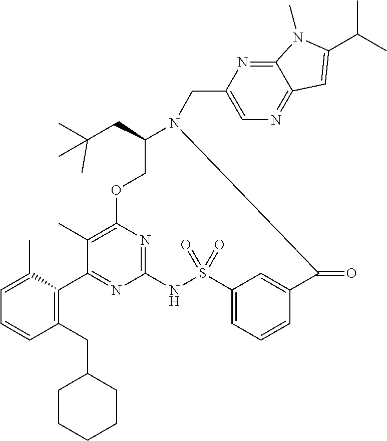 | | | +++ |
| I-179 | 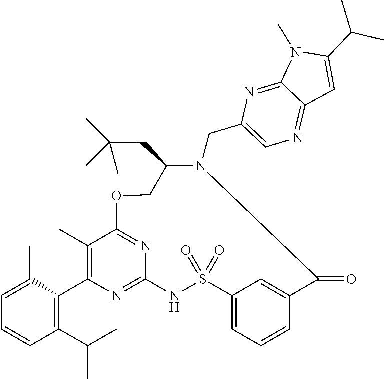 | | | + |
| I-182 | 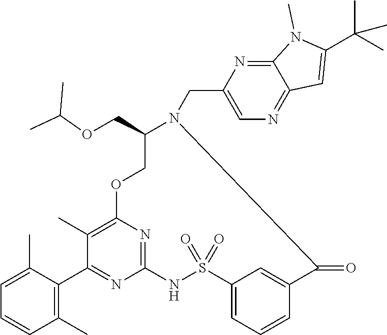 [SMILES: Cc1cccc(C)c1-c1nc2nc(c1C)OC[C@@H](COC(C)C)N(Cc1cnc3cc(C(C)(C)C)n(C)c3n1)C(=O)c1cccc(c1)S(=O)(=O)N2] | | +++ | +++ |
| I-185 | 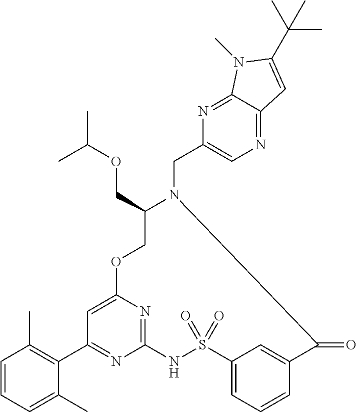 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](COC(C)C)CO2] | | | +++ |
| I-187 | 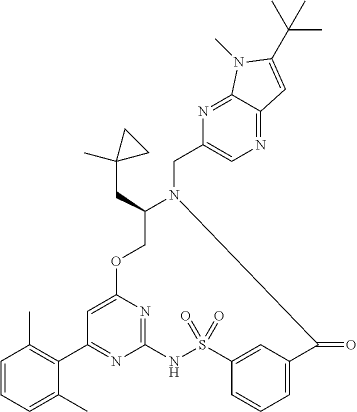 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](CC1(C)CC1)CO2] | | | +++ |
| I-188 | 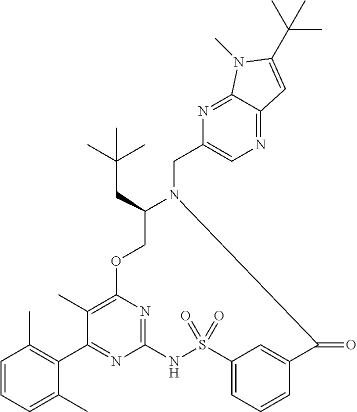 [SMILES: Cc1cccc(C)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2] | | +++ | +++ |
| I-193 | 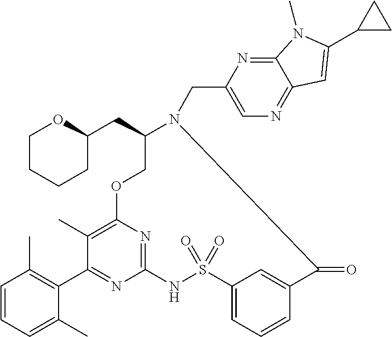 [SMILES: Cc1cccc(C)c1-c1nc2nc(c1C)OC[C@@H](C[C@H]1CCCCO1)N(Cc1cnc3cc(C4CC4)n(C)c3n1)C(=O)c1cccc(c1)S(=O)(=O)N2] | | | +++ |
| I-194 | 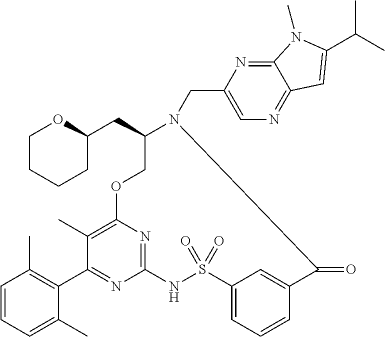 [SMILES: Cc1cccc(C)c1-c1nc2nc(c1C)OC[C@@H](C[C@H]1CCCCO1)N(Cc1cnc3cc(C(C)C)n(C)c3n1)C(=O)c1cccc(c1)S(=O)(=O)N2] | | | +++ |
| I-195 | 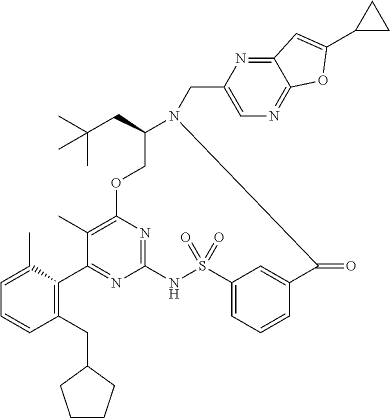 | | | +++ |
| I-196 | 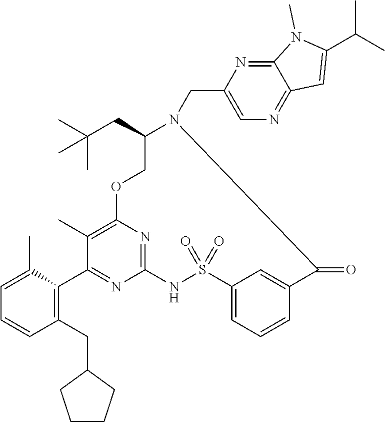 | | | +++ |
| I-197 | 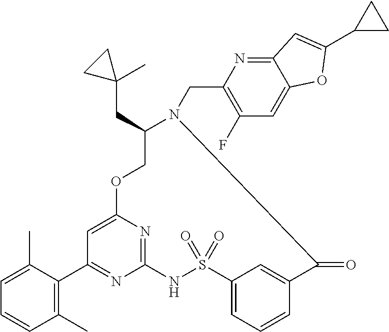 [SMILES: Cc1cccc(C)c1-c1cc2nc(n1)NS(=O)(=O)c1cccc(c1)C(=O)N(Cc1nc3cc(C4CC4)oc3cc1F)[C@H](CC1(C)CC1)CO2] | | | ++ |
| I-198 | 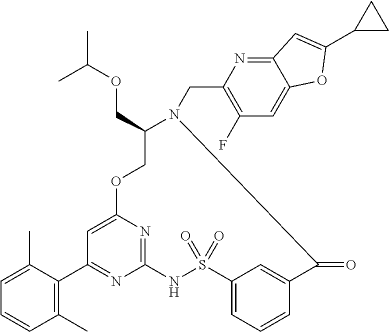 [SMILES: Cc1cccc(C)c1-c1cc2nc(n1)NS(=O)(=O)c1cccc(c1)C(=O)N(Cc1nc3cc(C4CC4)oc3cc1F)[C@H](COC(C)C)CO2] | | | ++ |
| I-199 | 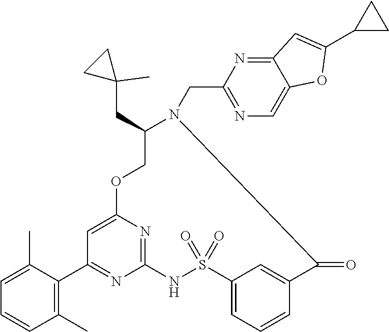 [SMILES: Cc1cccc(C)c1-c1cc2nc(n1)NS(=O)(=O)c1cccc(c1)C(=O)N(Cc1ncc3oc(C4CC4)cc3n1)[C@H](CC1(C)CC1)CO2] | | | + |
| I-202 | 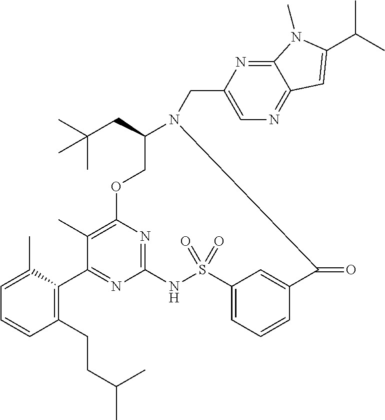 | | | +++ |
| I-204 | 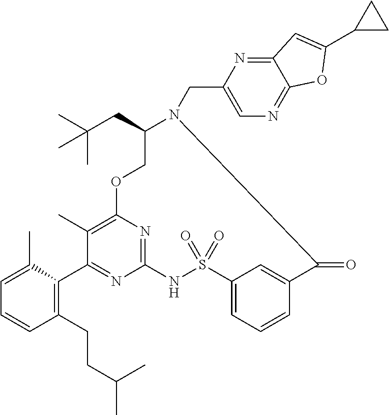 | | | +++ |
| I-206 | 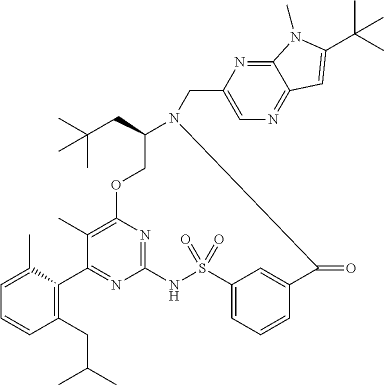 | | +++ | +++ |
| I-207 | 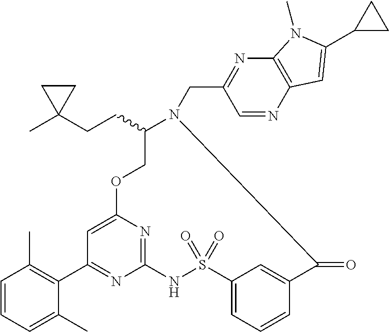 [SMILES: Cc1cccc(C)c1-c1cc2nc(n1)NS(=O)(=O)c1cccc(c1)C(=O)N(Cc1cnc3cc(C4CC4)n(C)c3n1)C(CCC1(C)CC1)CO2] | | | +++ |
| I-208 | 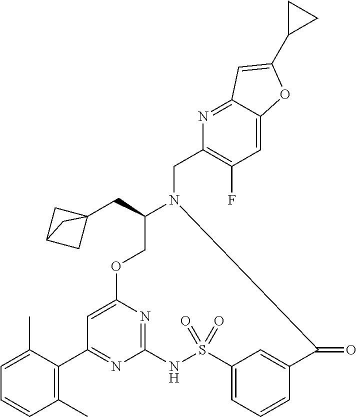 [SMILES: Cc1cccc(C)c1-c1cc2nc(n1)NS(=O)(=O)c1cccc(c1)C(=O)N(Cc1nc3cc(C4CC4)oc3cc1F)[C@H](CC13CC(C1)C3)CO2] | | | ++ |
| I-209 | 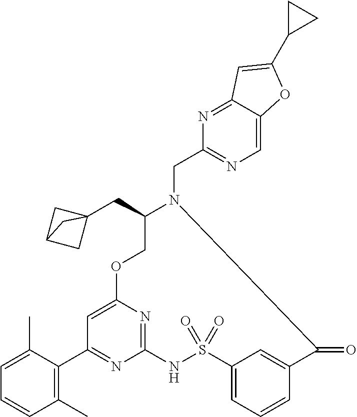 [SMILES: Cc1cccc(C)c1-c1cc2nc(n1)NS(=O)(=O)c1cccc(c1)C(=O)N(Cc1ncc3oc(C4CC4)cc3n1)[C@H](CC13CC(C1)C3)CO2] | | | ++ |
| I-211 | 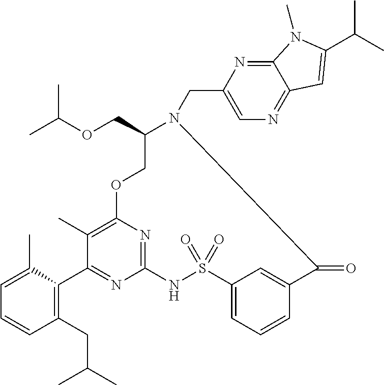 | | | +++ |
| I-213 | 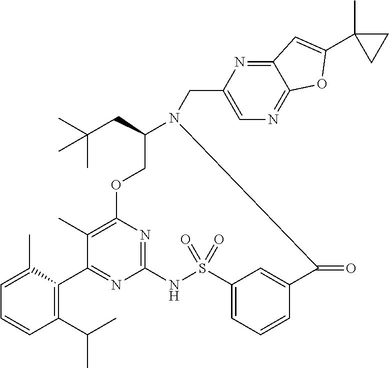 | | | +++ |
| I-217 | 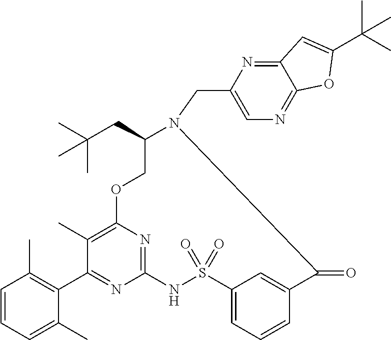 [SMILES: Cc1cccc(C)c1-c1nc2nc(c1C)OC[C@@H](CC(C)(C)C)N(Cc1cnc3oc(C(C)(C)C)cc3n1)C(=O)c1cccc(c1)S(=O)(=O)N2] | | | ++ |
| I-218 | 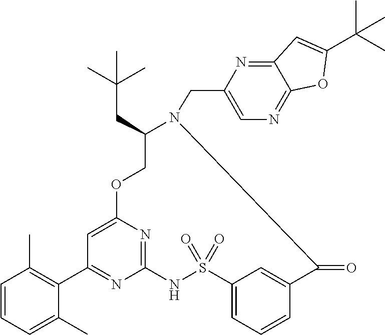 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC(C)(C)C)CO2] | | | +++ |
| I-222 | 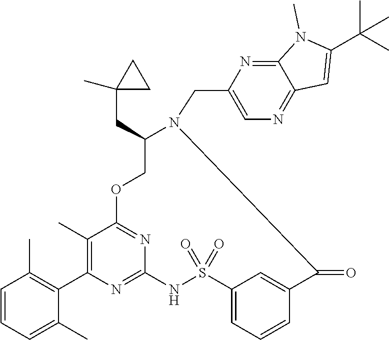 [SMILES: Cc1cccc(C)c1-c1nc2nc(c1C)OC[C@@H](CC1(C)CC1)N(Cc1cnc3cc(C(C)(C)C)n(C)c3n1)C(=O)c1cccc(c1)S(=O)(=O)N2] | | | +++ |
| I-227 | 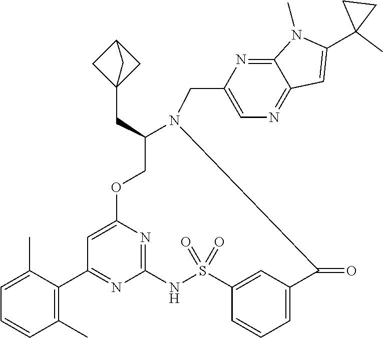 [SMILES: Cc1cccc(C)c1-c1cc2nc(n1)NS(=O)(=O)c1cccc(c1)C(=O)N(Cc1cnc3cc(C4(C)CC4)n(C)c3n1)[C@H](CC13CC(C1)C3)CO2] | | | +++ |
| I-228 | 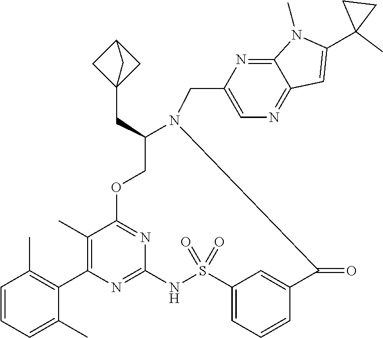 [SMILES: Cc1cccc(C)c1-c1nc2nc(c1C)OC[C@@H](CC13CC(C1)C3)N(Cc1cnc3cc(C4(C)CC4)n(C)c3n1)C(=O)c1cccc(c1)S(=O)(=O)N2] | | | +++ |
| I-231 | 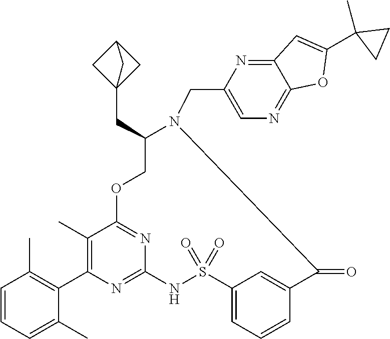 [SMILES: Cc1cccc(C)c1-c1nc2nc(c1C)OC[C@@H](CC13CC(C1)C3)N(Cc1cnc3oc(C4(C)CC4)cc3n1)C(=O)c1cccc(c1)S(=O)(=O)N2] | | | +++ |
| I-239 | 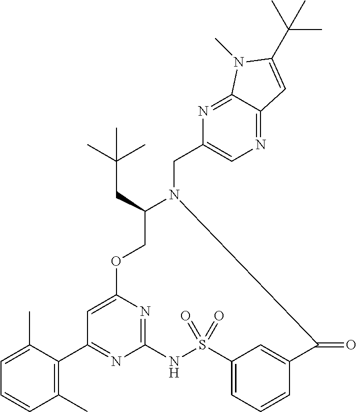 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](CC(C)(C)C)CO2] | | | +++ |
| I-240 | 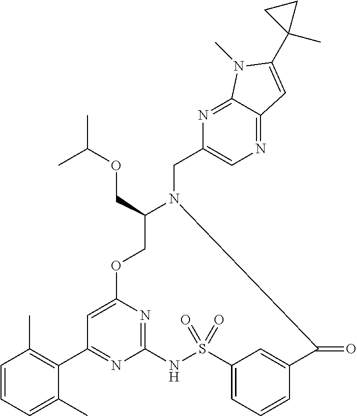 [SMILES: Cc1cccc(C)c1-c1cc2nc(n1)NS(=O)(=O)c1cccc(c1)C(=O)N(Cc1cnc3cc(C4(C)CC4)n(C)c3n1)[C@H](COC(C)C)CO2] | | | +++ |
| I-245 | 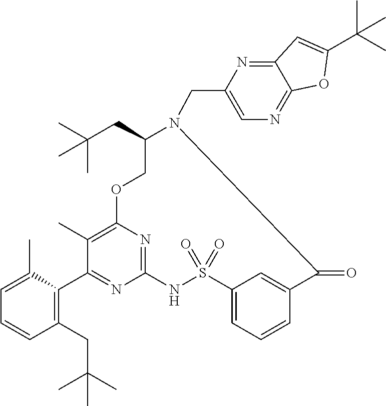 | | | +++ |
| I-247 | 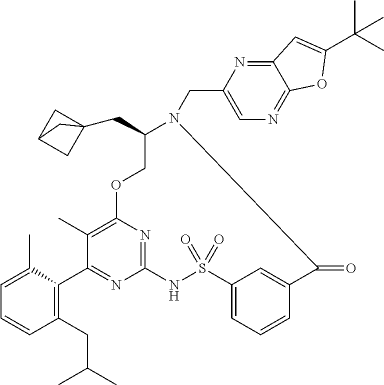 | | | +++ |
| I-249 | 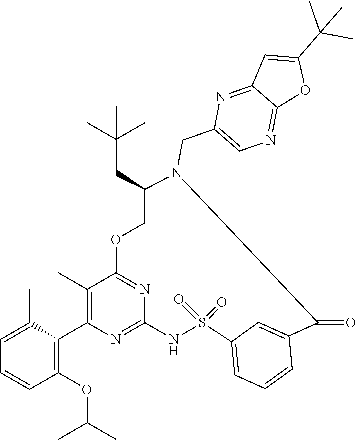 | | | ++ |
| I-251 | 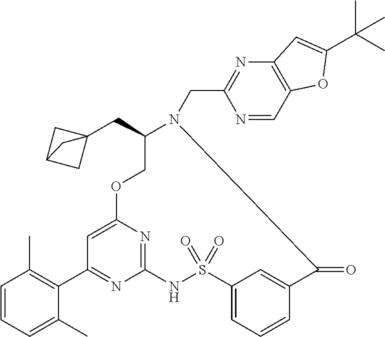 [SMILES: Cc1cccc(C)c1-c1cc2nc(n1)NS(=O)(=O)c1cccc(c1)C(=O)N(Cc1ncc3oc(C(C)(C)C)cc3n1)[C@H](CC13CC(C1)C3)CO2] | | | ++ |
| I-255 | 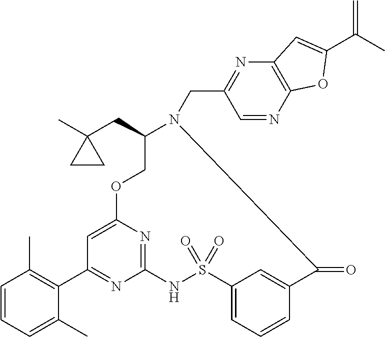 [SMILES: C=C(C)c1cc2nc(CN3C(=O)c4cccc(c4)S(=O)(=O)Nc4nc(cc(-c5c(C)cccc5C)n4)OC[C@H]3CC3(C)CC3)cnc2o1] | | | ++ |
| I-256 | 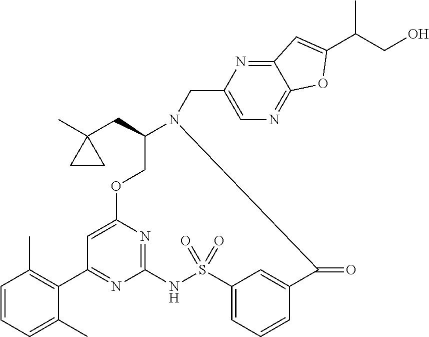 [SMILES: Cc1cccc(C)c1-c1cc2nc(n1)NS(=O)(=O)c1cccc(c1)C(=O)N(Cc1cnc3oc(C(C)CO)cc3n1)[C@H](CC1(C)CC1)CO2] | | | ++ |
| I-257 | 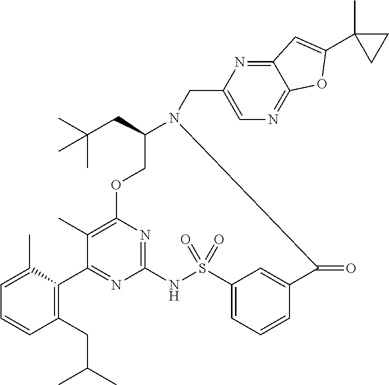 | | | ++ |
| I-259 | 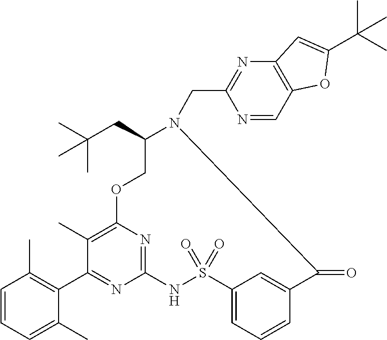 [SMILES: Cc1cccc(C)c1-c1nc2nc(c1C)OC[C@@H](CC(C)(C)C)N(Cc1ncc3oc(C(C)(C)C)cc3n1)C(=O)c1cccc(c1)S(=O)(=O)N2] | | | +++ |
| I-260 | 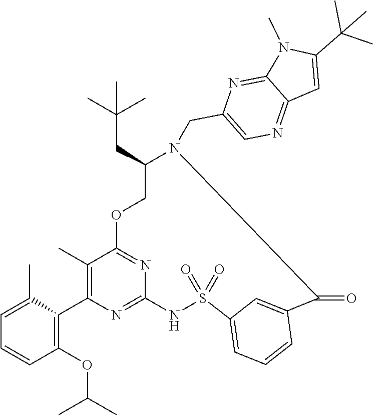 | | | +++ |
| I-262 | 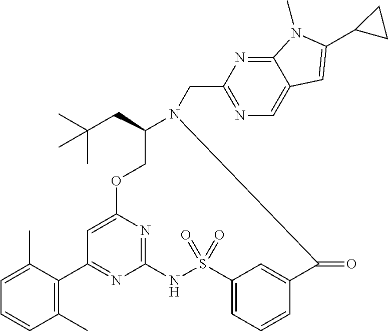 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC(C)(C)C)CO2] | | | ++ |
| I-263 | 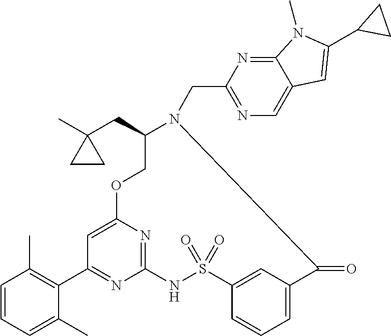 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC1(C)CC1)CO2] | | | ++ |
| I-264 | 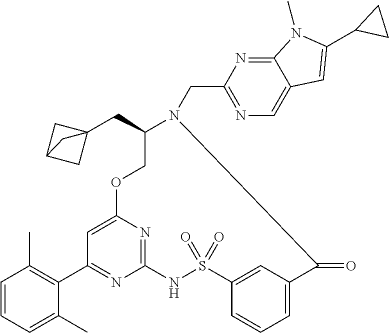 [SMILES: Cc1cccc(C)c1-c1cc2nc(n1)NS(=O)(=O)c1cccc(c1)C(=O)N(Cc1ncc3cc(C4CC4)n(C)c3n1)[C@H](CC13CC(C1)C3)CO2] | | | ++ |
| I-265 | 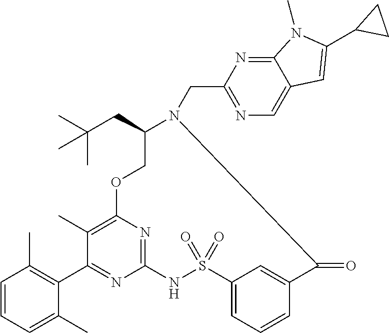 [SMILES: Cc1cccc(C)c1-c1nc2nc(c1C)OC[C@@H](CC(C)(C)C)N(Cc1ncc3cc(C4CC4)n(C)c3n1)C(=O)c1cccc(c1)S(=O)(=O)N2] | | | ++ |
| II-32 | 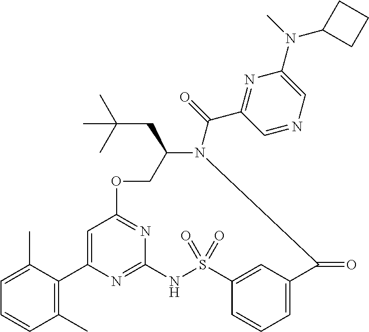 [SMILES: Cc1cccc(C)c1-c1cc2nc(n1)NS(=O)(=O)c1cccc(c1)C(=O)N(C(=O)c1cncc(N(C)C3CCC3)n1)[C@H](CC(C)(C)C)CO2] | +++ | | |
| II-36 | 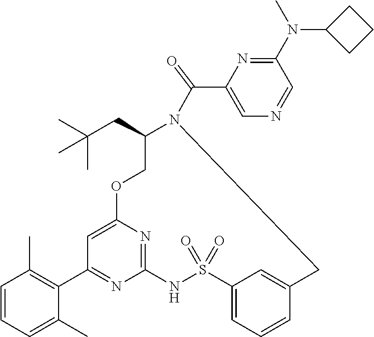 [SMILES: Cc1cccc(C)c1-c1cc2nc(n1)NS(=O)(=O)c1cccc(c1)CN(C(=O)c1cncc(N(C)C3CCC3)n1)[C@H](CC(C)(C)C)CO2] | ++ | | |
| III-1 | 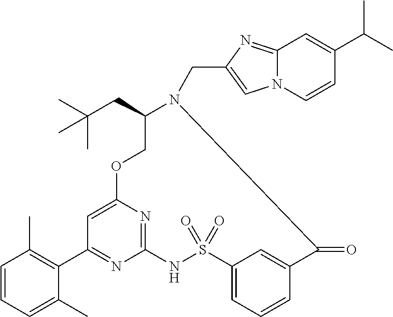 [SMILES: Cc1cccc(C)c1-c1cc2nc(n1)NS(=O)(=O)c1cccc(c1)C(=O)N(Cc1cn3ccc(C(C)C)cc3n1)[C@H](CC(C)(C)C)CO2] | +++ | | |
| III-7 | 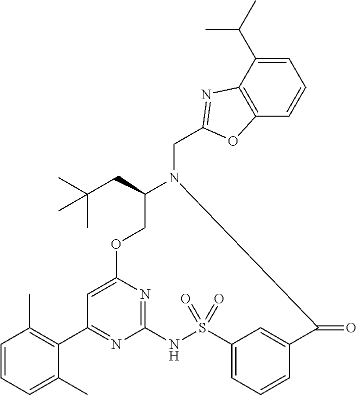 [SMILES: Cc1cccc(C)c1-c1cc2nc(n1)NS(=O)(=O)c1cccc(c1)C(=O)N(Cc1nc3c(C(C)C)cccc3o1)[C@H](CC(C)(C)C)CO2] | +++ | | |
| III-10 | 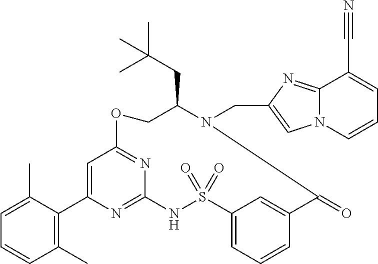 [SMILES: Cc1cccc(C)c1-c1cc2nc(n1)NS(=O)(=O)c1cccc(c1)C(=O)N(Cc1cn3cccc(C#N)c3n1)[C@H](CC(C)(C)C)CO2] | +++ | | |
| III-12 | 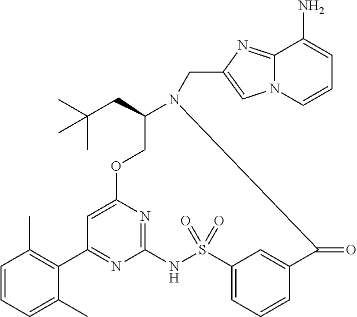 [SMILES: Cc1cccc(C)c1-c1cc2nc(n1)NS(=O)(=O)c1cccc(c1)C(=O)N(Cc1cn3cccc(N)c3n1)[C@H](CC(C)(C)C)CO2] | +++ | | |
| III-13 | 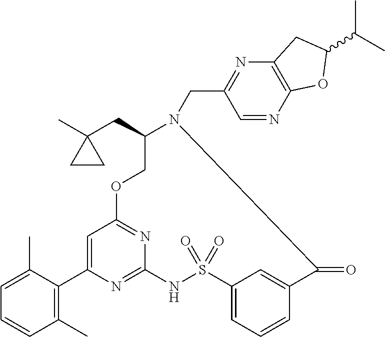 [SMILES: Cc1cccc(C)c1-c1cc2nc(n1)NS(=O)(=O)c1cccc(c1)C(=O)N(Cc1cnc3c(n1)CC(C(C)C)O3)[C@H](CC1(C)CC1)CO2] | +++ | | |
| III-14 | 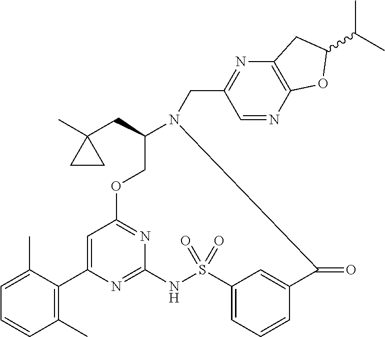 [SMILES: Cc1cccc(C)c1-c1cc2nc(n1)NS(=O)(=O)c1cccc(c1)C(=O)N(Cc1cnc3c(n1)CC(C(C)C)O3)[C@H](CC1(C)CC1)CO2] | +++ | | |
| III-18 | 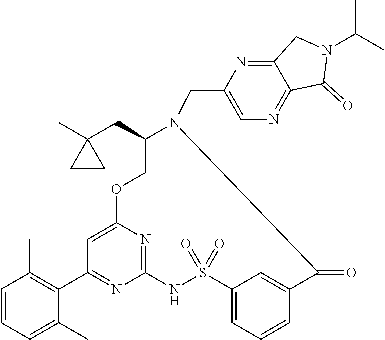 [SMILES: Cc1cccc(C)c1-c1cc2nc(n1)NS(=O)(=O)c1cccc(c1)C(=O)N(Cc1cnc3c(n1)CN(C(C)C)C3=O)[C@H](CC1(C)CC1)CO2] | | | ++ |
| III-21 | 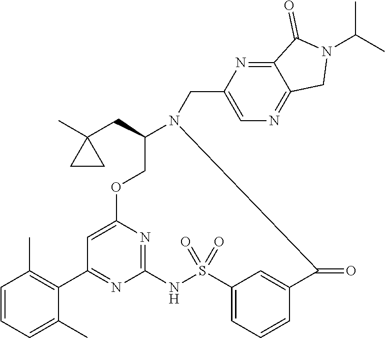 [SMILES: Cc1cccc(C)c1-c1cc2nc(n1)NS(=O)(=O)c1cccc(c1)C(=O)N(Cc1cnc3c(n1)C(=O)N(C(C)C)C3)[C@H](CC1(C)CC1)CO2] | | | ++ |

| | | B | B Max | C | C Max | D act. |
|---|---|---|---|---|---|---|
| Cmpd | | EC₅₀ | activity | EC₅₀ | activity | at 1 μM |
| Number | Structure | (μM) | (%) | (μM) | (%) | (%) |
| IV-1 | 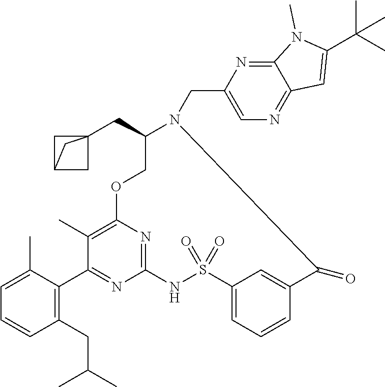 [SMILES: Cc1cccc(CC(C)C)c1-c1nc2nc(c1C)OC[C@@H](CC13CC(C1)C3)N(Cc1cnc3cc(C(C)(C)C)n(C)c3n1)C(=O)c1cccc(c1)S(=O)(=O)N2] | +++ | +++ | | | +++ |
| IV-2 | 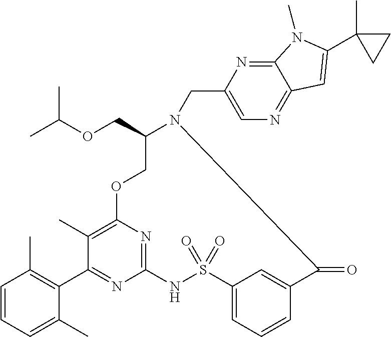 [SMILES: Cc1cccc(C)c1-c1nc2nc(c1C)OC[C@@H](COC(C)C)N(Cc1cnc3cc(C4(C)CC4)n(C)c3n1)C(=O)c1cccc(c1)S(=O)(=O)N2] | | | +++ | +++ | +++ |
| IV-3 | 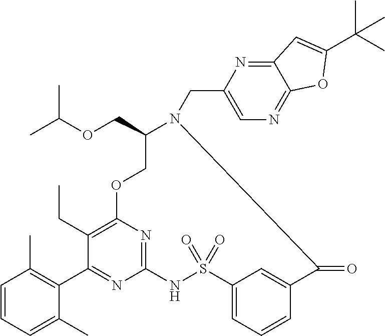 [SMILES: CCc1c2nc(nc1-c1c(C)cccc1C)NS(=O)(=O)c1cccc(c1)C(=O)N(Cc1cnc3oc(C(C)(C)C)cc3n1)[C@H](COC(C)C)CO2] | | | | | +++ |
| IV-4 | 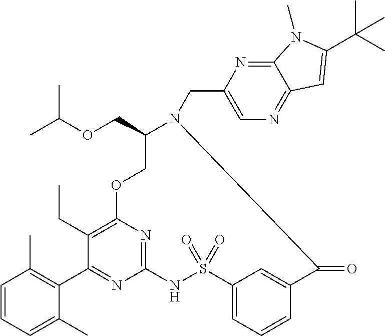 [SMILES: CCc1c2nc(nc1-c1c(C)cccc1C)NS(=O)(=O)c1cccc(c1)C(=O)N(Cc1cnc3cc(C(C)(C)C)n(C)c3n1)[C@H](COC(C)C)CO2] | | | +++ | +++ | +++ |
| IV-5 | 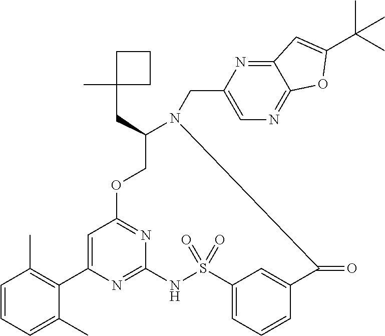 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1(C)CCC1)CO2] | | | ++ | +++ | +++ |
| IV-6 | 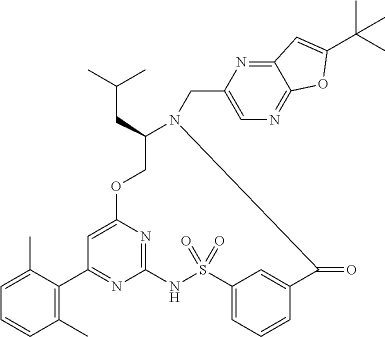 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC(C)C)CO2] | | | | | +++ |
| IV-7 | 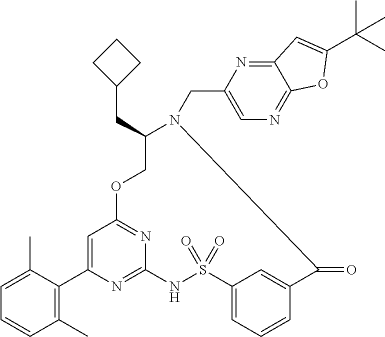 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCC1)CO2] | | | | | +++ |
| IV-8 | 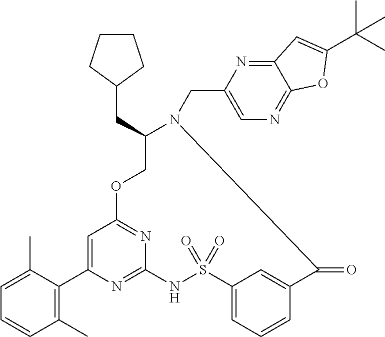 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCCC1)CO2] | | | | | +++ |
| IV-9 | 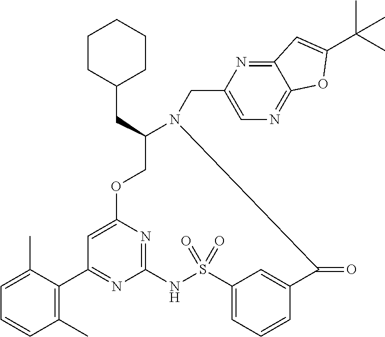 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC1CCCCC1)CO2] | | | | | +++ |
| IV-10 | 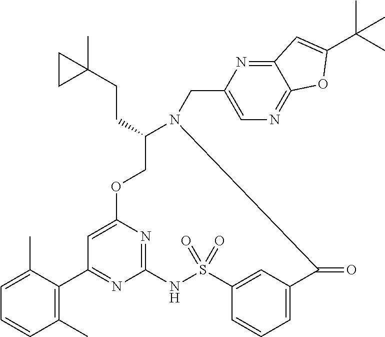 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CCC1(C)CC1)CO2] | | | | | +++ |
| IV-11 | 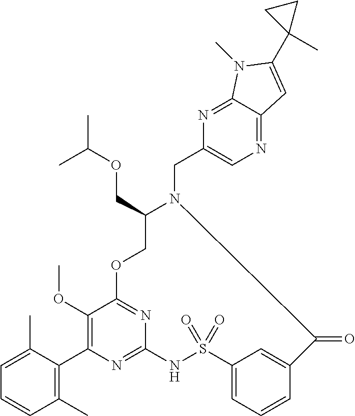 [SMILES: COc1c2nc(nc1-c1c(C)cccc1C)NS(=O)(=O)c1cccc(c1)C(=O)N(Cc1cnc3cc(C4(C)CC4)n(C)c3n1)[C@H](COC(C)C)CO2] | | | +++ | +++ | +++ |
| IV-12 | 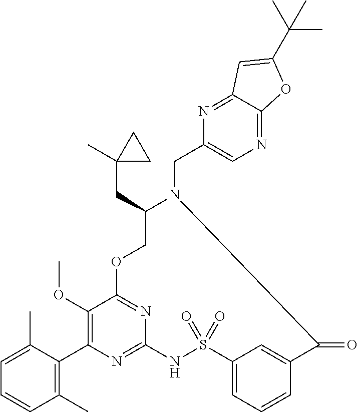 [SMILES: COc1c2nc(nc1-c1c(C)cccc1C)NS(=O)(=O)c1cccc(c1)C(=O)N(Cc1cnc3oc(C(C)(C)C)cc3n1)[C@H](CC1(C)CC1)CO2] | | | ++ | +++ | +++ |
| IV-13 | 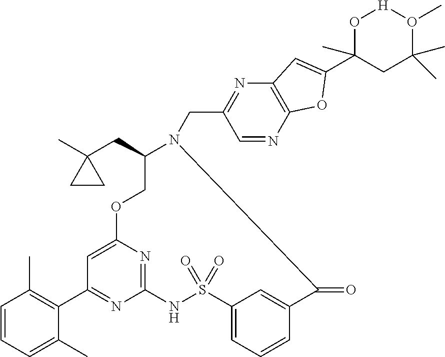 | | | | | +++ |
| IV-14 | 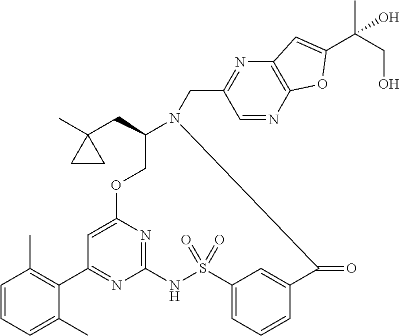 [SMILES: Cc1cccc(C)c1-c1cc2nc(n1)NS(=O)(=O)c1cccc(c1)C(=O)N(Cc1cnc3oc([C@](C)(O)CO)cc3n1)[C@H](CC1(C)CC1)CO2] | | | | | +++ |
| IV-15 | 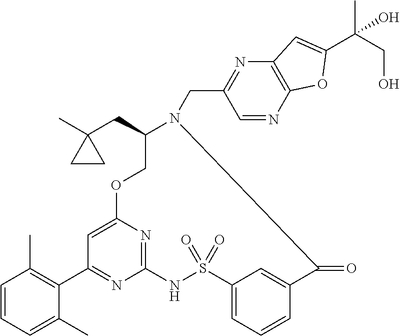 [SMILES: Cc1cccc(C)c1-c1cc2nc(n1)NS(=O)(=O)c1cccc(c1)C(=O)N(Cc1cnc3oc([C@](C)(O)CO)cc3n1)[C@H](CC1(C)CC1)CO2] | | | | | +++ |
| IV-16 | 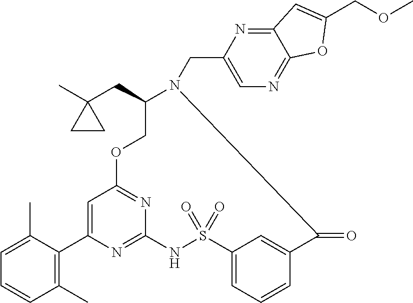 [SMILES: COCc1cc2nc(CN3C(=O)c4cccc(c4)S(=O)(=O)Nc4nc(cc(-c5c(C)cccc5C)n4)OC[C@H]3CC3(C)CC3)cnc2o1] | | | | | +++ |
| IV-17 | 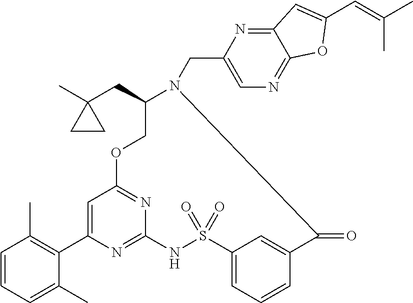 [SMILES: CC(C)=Cc1cc2nc(CN3C(=O)c4cccc(c4)S(=O)(=O)Nc4nc(cc(-c5c(C)cccc5C)n4)OC[C@H]3CC3(C)CC3)cnc2o1] | | | | | +++ |
| IV-18 | 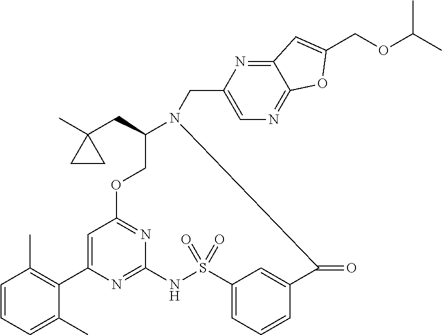 [SMILES: Cc1cccc(C)c1-c1cc2nc(n1)NS(=O)(=O)c1cccc(c1)C(=O)N(Cc1cnc3oc(COC(C)C)cc3n1)[C@H](CC1(C)CC1)CO2] | | | ++ | +++ | +++ |
| IV-19 | 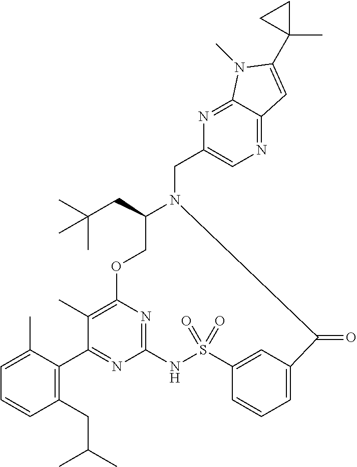 [SMILES: Cc1cccc(CC(C)C)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] | | | +++ | +++ | +++ |
| IV-20 | 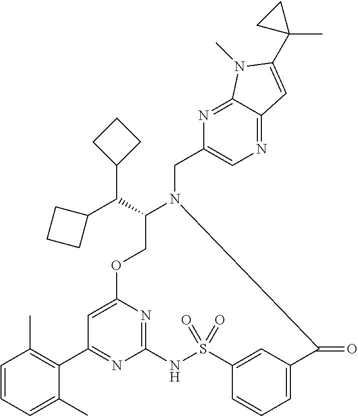 [SMILES: Cc1cccc(C)c1-c1cc2nc(n1)NS(=O)(=O)c1cccc(c1)C(=O)N(Cc1cnc3cc(C4(C)CC4)n(C)c3n1)[C@@H](C(C1CCC1)C1CCC1)CO2] | | | +++ | +++ | +++ |
| IV-21 | 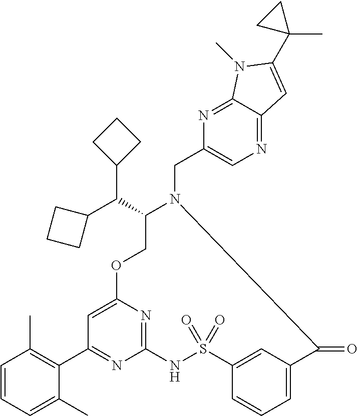 [SMILES: Cc1cccc(C)c1-c1cc2nc(n1)NS(=O)(=O)c1cccc(c1)C(=O)N(Cc1cnc3cc(C4(C)CC4)n(C)c3n1)[C@@H](C(C1CCC1)C1CCC1)CO2] | | | | | ++ |
| IV-23 | 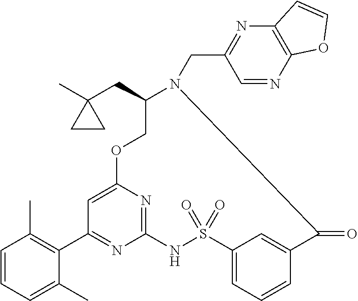 [SMILES: Cc1cccc(C)c1-c1cc2nc(n1)NS(=O)(=O)c1cccc(c1)C(=O)N(Cc1cnc3occc3n1)[C@H](CC1(C)CC1)CO2] | | | | | +++ |
| IV-24 | 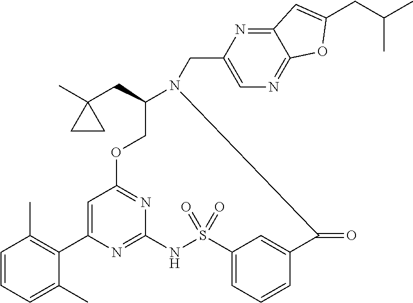 [SMILES: Cc1cccc(C)c1-c1cc2nc(n1)NS(=O)(=O)c1cccc(c1)C(=O)N(Cc1cnc3oc(CC(C)C)cc3n1)[C@H](CC1(C)CC1)CO2] | | | + | +++ | +++ |
| IV-25 | 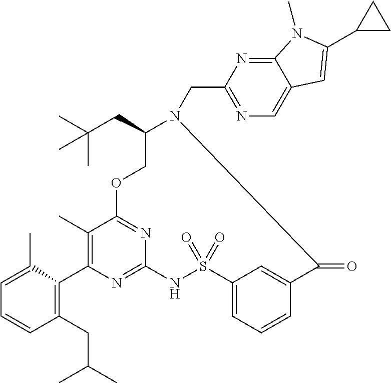 | | | ++ | +++ | |
| IV-26 | 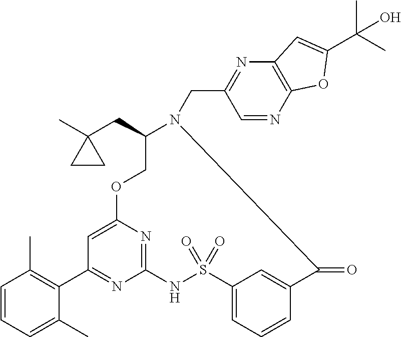 [SMILES: Cc1cccc(C)c1-c1cc2nc(n1)NS(=O)(=O)c1cccc(c1)C(=O)N(Cc1cnc3oc(C(C)(C)O)cc3n1)[C@H](CC1(C)CC1)CO2] | | | +++ | +++ | |
| IV-27 | 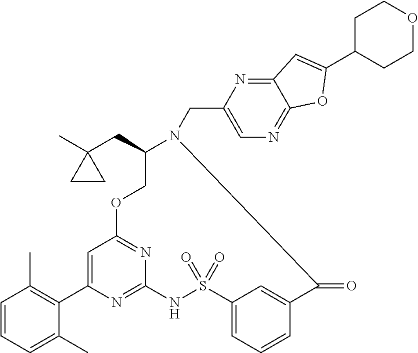 [SMILES: Cc1cccc(C)c1-c1cc2nc(n1)NS(=O)(=O)c1cccc(c1)C(=O)N(Cc1cnc3oc(C4CCOCC4)cc3n1)[C@H](CC1(C)CC1)CO2] | | | + | +++ | +++ |
| IV-28 | 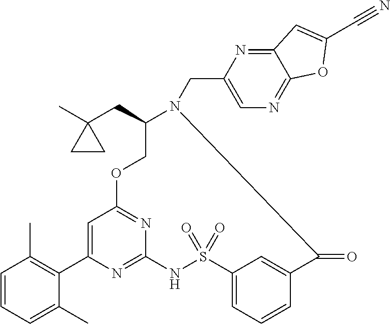 [SMILES: Cc1cccc(C)c1-c1cc2nc(n1)NS(=O)(=O)c1cccc(c1)C(=O)N(Cc1cnc3oc(C#N)cc3n1)[C@H](CC1(C)CC1)CO2] | | | | | +++ |
| IV-29 | 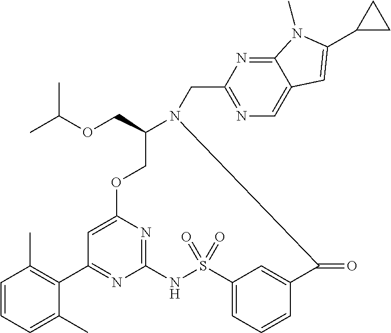 [SMILES: Cc1cccc(C)c1-c1cc2nc(n1)NS(=O)(=O)c1cccc(c1)C(=O)N(Cc1ncc3cc(C4CC4)n(C)c3n1)[C@H](COC(C)C)CO2] | | | | | +++ |
| IV-30 | 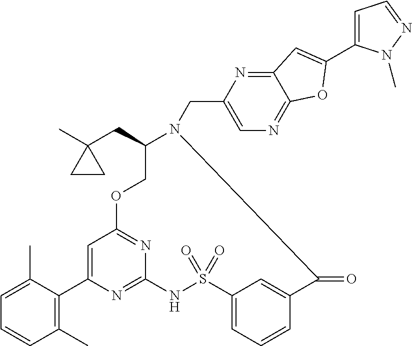 [SMILES: Cc1cccc(C)c1-c1cc2nc(n1)NS(=O)(=O)c1cccc(c1)C(=O)N(Cc1cnc3oc(-c4ccnn4C)cc3n1)[C@H](CC1(C)CC1)CO2] | | | + | +++ | +++ |
| IV-31 | 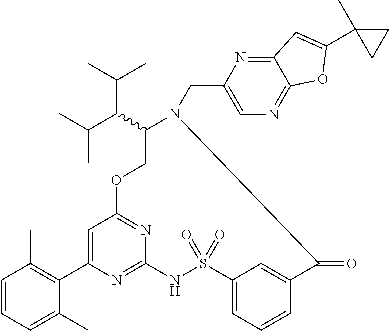 [SMILES: Cc1cccc(C)c1-c1cc2nc(n1)NS(=O)(=O)c1cccc(c1)C(=O)N(Cc1cnc3oc(C4(C)CC4)cc3n1)C(C(C(C)C)C(C)C)CO2] | | | +++ | +++ | +++ |
| IV-32 | 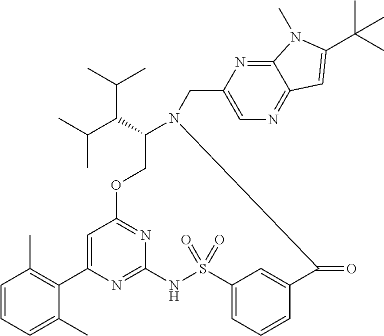 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](C(C(C)C)C(C)C)CO2] | | | +++ | +++ | +++ |
| IV-33 | 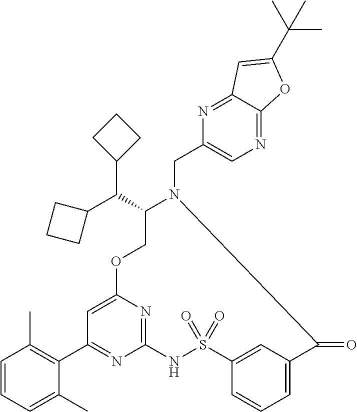 [SMILES: Cc1cccc(C)c1-c1cc2nc(n1)NS(=O)(=O)c1cccc(c1)C(=O)N(Cc1cnc3oc(C(C)(C)C)cc3n1)[C@@H](C(C1CCC1)C1CCC1)CO2] | | | + | +++ | +++ |
| IV-34 | 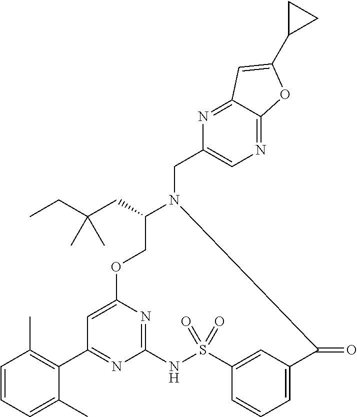 [SMILES: CCC(C)(C)C[C@H]1COc2cc(-c3c(C)cccc3C)nc(n2)NS(=O)(=O)c2cccc(c2)C(=O)N1Cc1cnc2oc(C3CC3)cc2n1] | | | ++ | +++ | +++ |
| IV-35 | 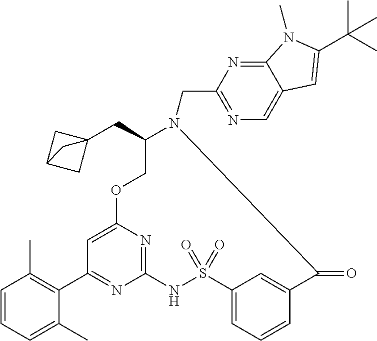 [SMILES: Cc1cccc(C)c1-c1cc2nc(n1)NS(=O)(=O)c1cccc(c1)C(=O)N(Cc1ncc3cc(C(C)(C)C)n(C)c3n1)[C@H](CC13CC(C1)C3)CO2] | | | +++ | +++ | +++ |
| IV-36 | 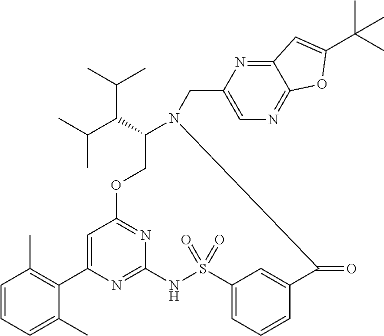 [SMILES: Cc1cccc(C)c1-c1cc2nc(n1)NS(=O)(=O)c1cccc(c1)C(=O)N(Cc1cnc3oc(C(C)(C)C)cc3n1)[C@@H](C(C(C)C)C(C)C)CO2] | | | +++ | +++ | +++ |
| IV-37 | 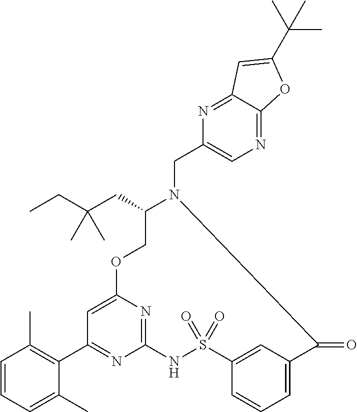 [SMILES: CCC(C)(C)C[C@H]1COc2cc(-c3c(C)cccc3C)nc(n2)NS(=O)(=O)c2cccc(c2)C(=O)N1Cc1cnc2oc(C(C)(C)C)cc2n1] | | | ++ | +++ | +++ |
| IV-38 | 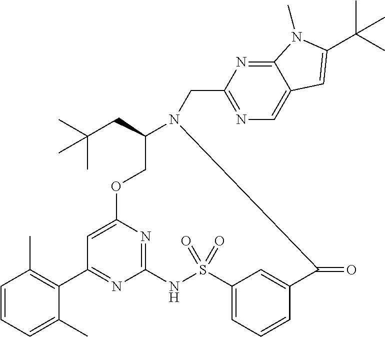 [SMILES: Cc1cccc(C)c1-c1cc2nc(n1)NS(=O)(=O)c1cccc(c1)C(=O)N(Cc1ncc3cc(C(C)(C)C)n(C)c3n1)[C@H](CC(C)(C)C)CO2] | | | ++ | +++ | +++ |
| IV-39 | 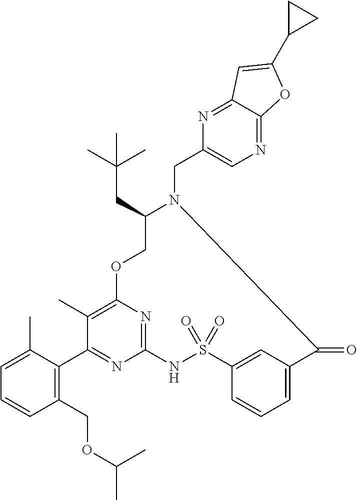 [SMILES: Cc1cccc(COC(C)C)c1-c1nc2nc(c1C)OC[C@@H](CC(C)(C)C)N(Cc1cnc3oc(C4CC4)cc3n1)C(=O)c1cccc(c1)S(=O)(=O)N2] | | | ++ | +++ | +++ |
| IV-40 | 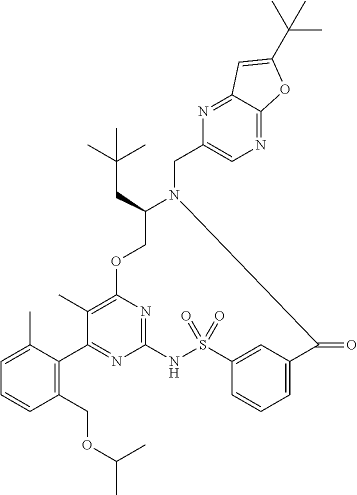 [SMILES: Cc1cccc(COC(C)C)c1-c1nc2nc(c1C)OC[C@@H](CC(C)(C)C)N(Cc1cnc3oc(C(C)(C)C)cc3n1)C(=O)c1cccc(c1)S(=O)(=O)N2] | | | +++ | +++ | +++ |
| IV-41 | 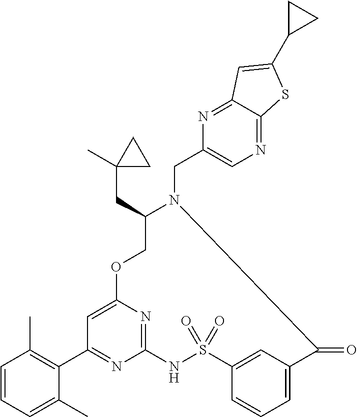 [SMILES: Cc1cccc(C)c1-c1cc2nc(n1)NS(=O)(=O)c1cccc(c1)C(=O)N(Cc1cnc3sc(C4CC4)cc3n1)[C@H](CC1(C)CC1)CO2] | | | | | +++ |
| IV-42 | 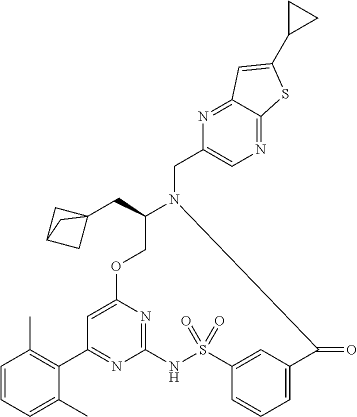 [SMILES: Cc1cccc(C)c1-c1cc2nc(n1)NS(=O)(=O)c1cccc(c1)C(=O)N(Cc1cnc3sc(C4CC4)cc3n1)[C@H](CC13CC(C1)C3)CO2] | | | ++ | +++ | |
| IV-43 | 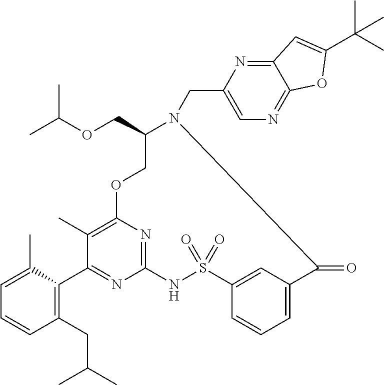 | | | ++ | +++ | |
| IV-44 | 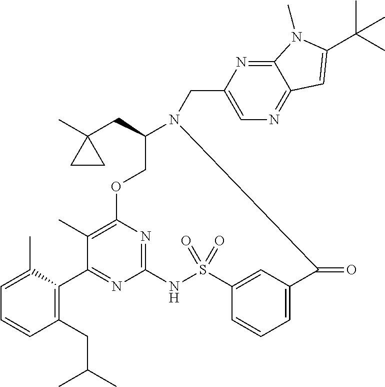 | | | ++ | +++ | +++ |
| IV-45 | 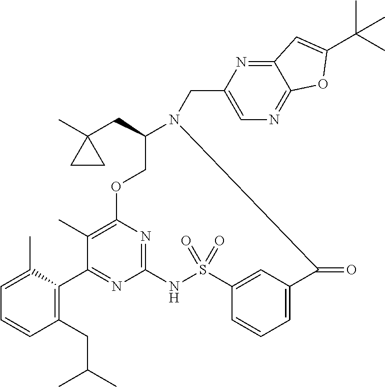 | | | ++ | +++ | +++ |
| IV-46 | 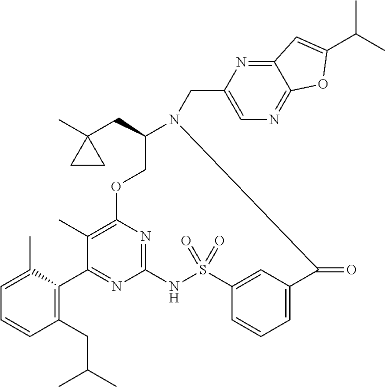 | | | +++ | +++ | +++ |
| IV-47 | 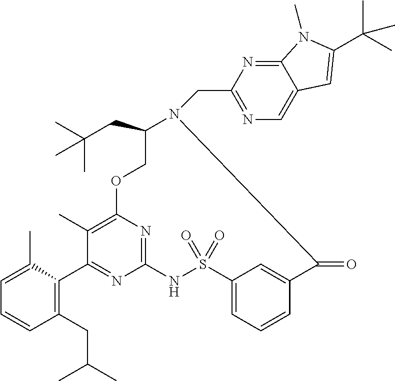 | | | ++ | +++ | +++ |
| IV-48 | 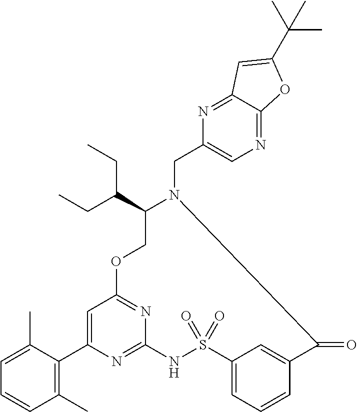 [SMILES: CCC(CC)[C@@H]1COc2cc(-c3c(C)cccc3C)nc(n2)NS(=O)(=O)c2cccc(c2)C(=O)N1Cc1cnc2oc(C(C)(C)C)cc2n1] | | | ++ | +++ | +++ |
| IV-49 | 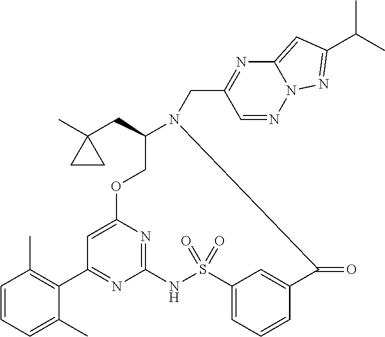 [SMILES: Cc1cccc(C)c1-c1cc2nc(n1)NS(=O)(=O)c1cccc(c1)C(=O)N(Cc1cnn3nc(C(C)C)cc3n1)[C@H](CC1(C)CC1)CO2] | | | ++ | +++ | |
| IV-50 | 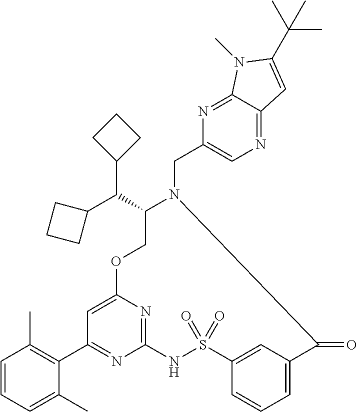 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](C(C1CCC1)C1CCC1)CO2] | | | +++ | +++ | |
| IV-51 | 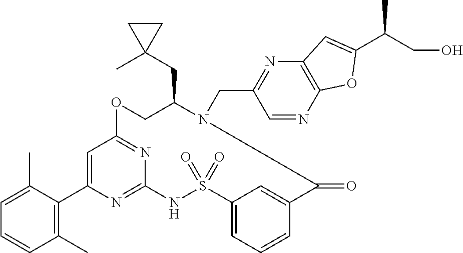 [SMILES: Cc1cccc(C)c1-c1cc2nc(n1)NS(=O)(=O)c1cccc(c1)C(=O)N(Cc1cnc3oc([C@@H](C)CO)cc3n1)[C@H](CC1(C)CC1)CO2] | | | | | |
| IV-52 | 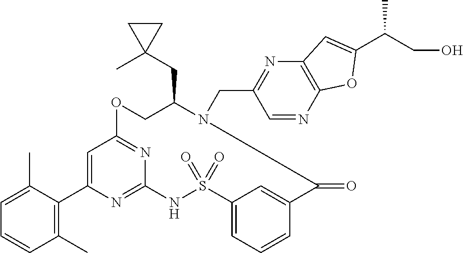 [SMILES: Cc1cccc(C)c1-c1cc2nc(n1)NS(=O)(=O)c1cccc(c1)C(=O)N(Cc1cnc3oc([C@H](C)CO)cc3n1)[C@H](CC1(C)CC1)CO2] | | | + | +++ | |
| IV-53 | 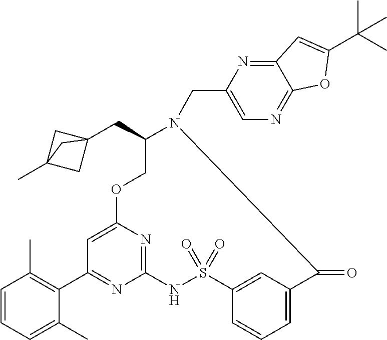 [SMILES: Cc1cccc(C)c1-c1cc2nc(n1)NS(=O)(=O)c1cccc(c1)C(=O)N(Cc1cnc3oc(C(C)(C)C)cc3n1)[C@H](CC13CC(C)(C1)C3)CO2] | | | ++ | +++ | |
| IV-54 | 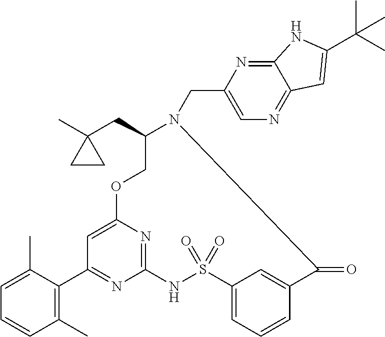 [SMILES: Cc1cccc(C)c1-c1cc2nc(n1)NS(=O)(=O)c1cccc(c1)C(=O)N(Cc1cnc3cc(C(C)(C)C)[nH]c3n1)[C@H](CC1(C)CC1)CO2] | | | + | +++ | |
| IV-55 | 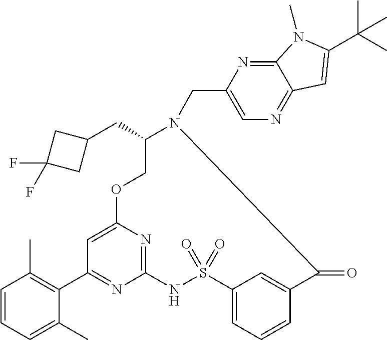 [SMILES: Cc1cccc(C)c1-c1cc2nc(n1)NS(=O)(=O)c1cccc(c1)C(=O)N(Cc1cnc3cc(C(C)(C)C)n(C)c3n1)[C@@H](CC1CC(F)(F)C1)CO2] | | | +++ | +++ | |
| IV-56 | 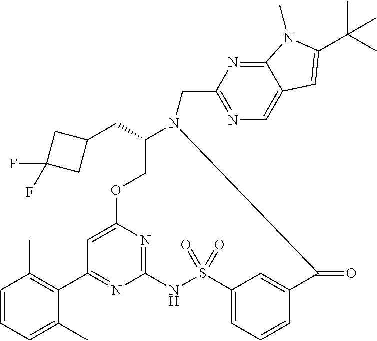 [SMILES: Cc1cccc(C)c1-c1cc2nc(n1)NS(=O)(=O)c1cccc(c1)C(=O)N(Cc1ncc3cc(C(C)(C)C)n(C)c3n1)[C@@H](CC1CC(F)(F)C1)CO2] | | | | | |
| IV-57 | 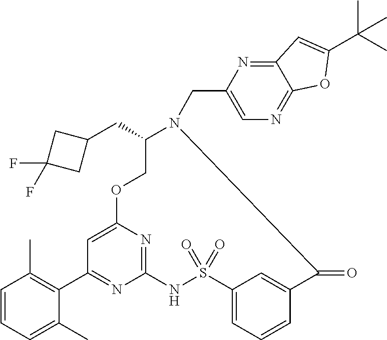 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CC1CC(F)(F)C1)CO2] | | | + | +++ | |
| IV-58 | 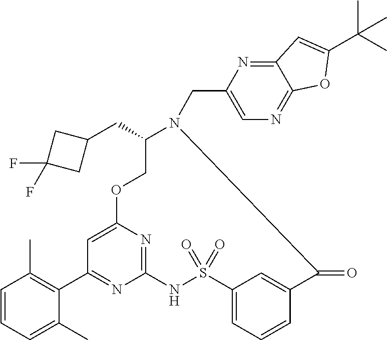 [SMILES: Cc1cccc(C)c1-c1cc2nc(n1)NS(=O)(=O)c1cccc(c1)C(=O)N(Cc1cnc3oc(C(C)(C)C)cc3n1)[C@@H](CC1CC(F)(F)C1)CO2] | | | | | |
| IV-59 | 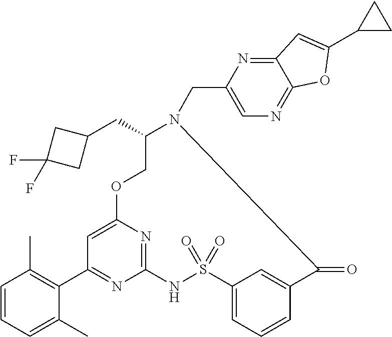 [SMILES: Cc1cccc(C)c1-c1cc2nc(n1)NS(=O)(=O)c1cccc(c1)C(=O)N(Cc1cnc3oc(C4CC4)cc3n1)[C@@H](CC1CC(F)(F)C1)CO2] | | | | | |
| IV-60 | 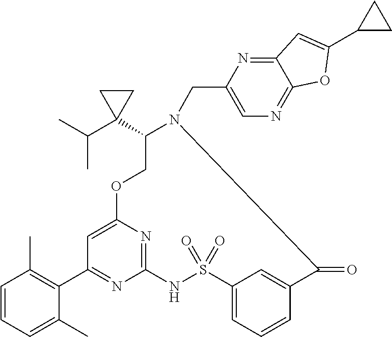 [SMILES: Cc1cccc(C)c1-c1cc2nc(n1)NS(=O)(=O)c1cccc(c1)C(=O)N(Cc1cnc3oc(C4CC4)cc3n1)[C@@H](C1(C(C)C)CC1)CO2] | | | | | |
| IV-61 | 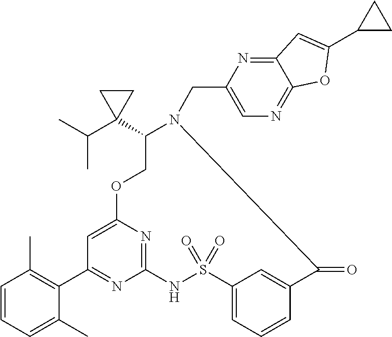 [SMILES: Cc1cccc(C)c1-c1cc2nc(n1)NS(=O)(=O)c1cccc(c1)C(=O)N(Cc1cnc3oc(C4CC4)cc3n1)[C@@H](C1(C(C)C)CC1)CO2] | | | ++ | +++ | |
| IV-62 | 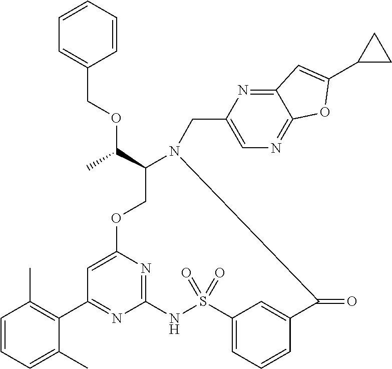 [SMILES: Cc1cccc(C)c1-c1cc2nc(n1)NS(=O)(=O)c1cccc(c1)C(=O)N(Cc1cnc3oc(C4CC4)cc3n1)[C@H]([C@H](C)OCc1ccccc1)CO2] | | | + | +++ | |
| IV-63 | 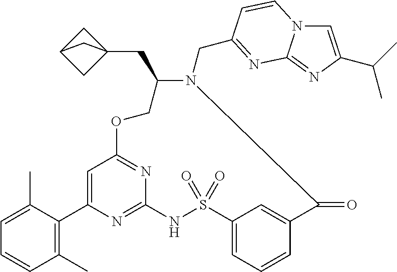 [SMILES: Cc1cccc(C)c1-c1cc2nc(n1)NS(=O)(=O)c1cccc(c1)C(=O)N(Cc1ccn3cc(C(C)C)nc3n1)[C@H](CC13CC(C1)C3)CO2] | | | +++ | +++ | |
| IV-64 | 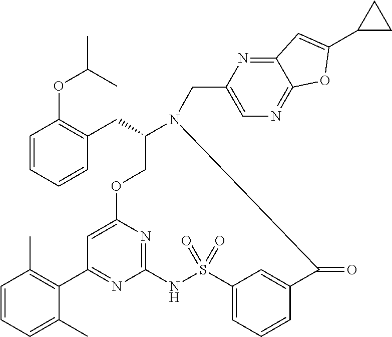 [SMILES: Cc1cccc(C)c1-c1cc2nc(n1)NS(=O)(=O)c1cccc(c1)C(=O)N(Cc1cnc3oc(C4CC4)cc3n1)[C@@H](Cc1ccccc1OC(C)C)CO2] | | | | | |
| IV-65 | 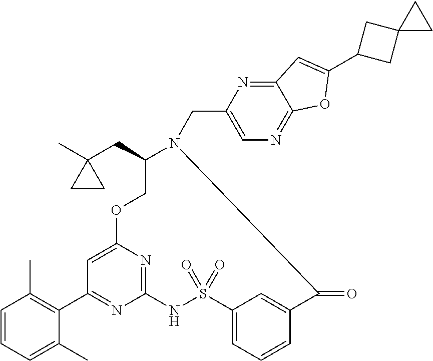 [SMILES: Cc1cccc(C)c1-c1cc2nc(n1)NS(=O)(=O)c1cccc(c1)C(=O)N(Cc1cnc3oc(C4CC5(CC5)C4)cc3n1)[C@H](CC1(C)CC1)CO2] | | | ++ | +++ | |
| IV-66 | 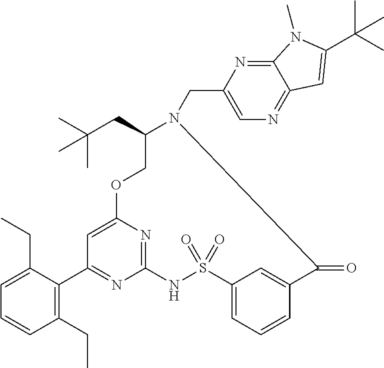 [SMILES: CCc1cccc(CC)c1-c1cc2nc(n1)NS(=O)(=O)c1cccc(c1)C(=O)N(Cc1cnc3cc(C(C)(C)C)n(C)c3n1)[C@H](CC(C)(C)C)CO2] | | | +++ | +++ | |
| IV-67 | 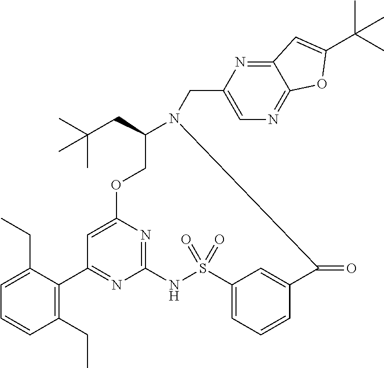 [SMILES: CCc1cccc(CC)c1-c1cc2nc(n1)NS(=O)(=O)c1cccc(c1)C(=O)N(Cc1cnc3oc(C(C)(C)C)cc3n1)[C@H](CC(C)(C)C)CO2] | | | + | +++ | |
| IV-68 | 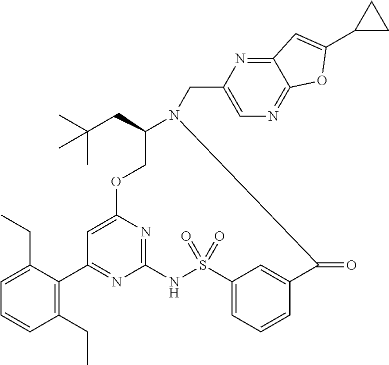 [SMILES: CCc1cccc(CC)c1-c1cc2nc(n1)NS(=O)(=O)c1cccc(c1)C(=O)N(Cc1cnc3oc(C4CC4)cc3n1)[C@H](CC(C)(C)C)CO2] | | | ++ | +++ | |
| IV-69 | 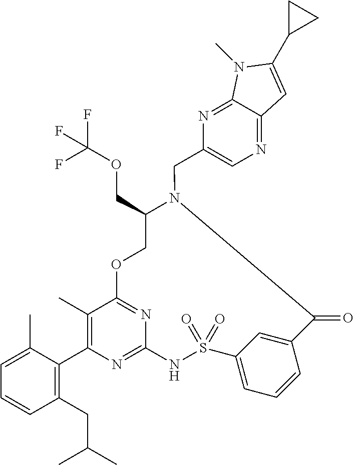 [SMILES: Cc1cccc(CC(C)C)c1-c1nc2nc(c1C)OC[C@@H](COC(F)(F)F)N(Cc1cnc3cc(C4CC4)n(C)c3n1)C(=O)c1cccc(c1)S(=O)(=O)N2] | | | ++ | +++ | |
| IV-70 | 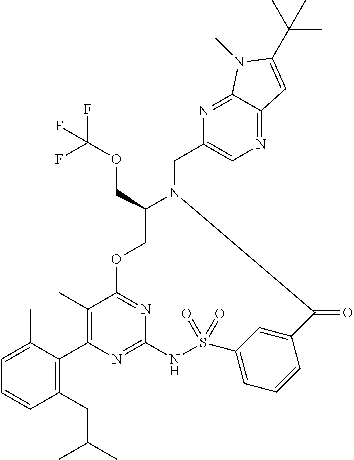 [SMILES: Cc1cccc(CC(C)C)c1-c1nc2nc(c1C)OC[C@@H](COC(F)(F)F)N(Cc1cnc3cc(C(C)(C)C)n(C)c3n1)C(=O)c1cccc(c1)S(=O)(=O)N2] | | | ++ | +++ | |
| IV-71 | 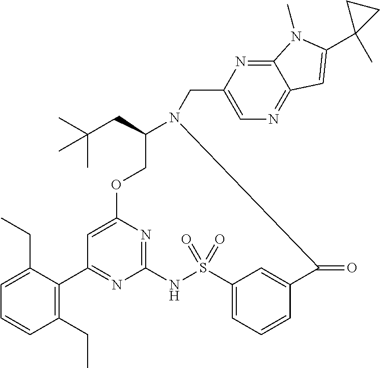 [SMILES: CCc1cccc(CC)c1-c1cc2nc(n1)NS(=O)(=O)c1cccc(c1)C(=O)N(Cc1cnc3cc(C4(C)CC4)n(C)c3n1)[C@H](CC(C)(C)C)CO2] | | | ++ | +++ | |
| IV-72 | 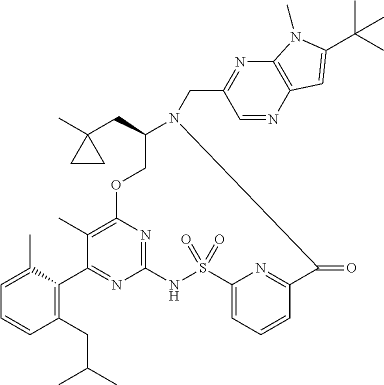 | | | ++ | +++ | |
| IV-73 | 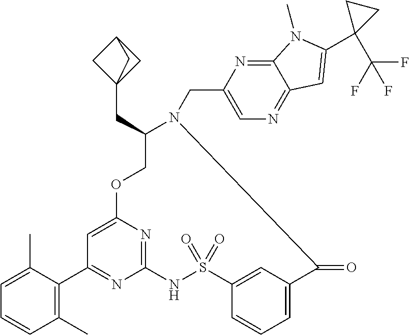 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](CC13CC(C1)C3)CO2] | | | +++ | +++ | |
| IV-74 | 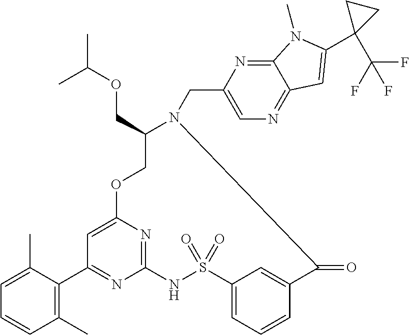 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](COC(C)C)CO2] | | | ++ | +++ | |
| IV-75 | 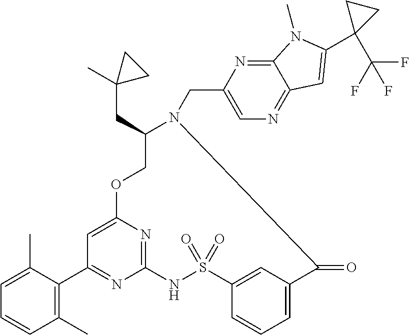 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](CC1(C)CC1)CO2] | | | ++ | ++ | |
| IV-76 | 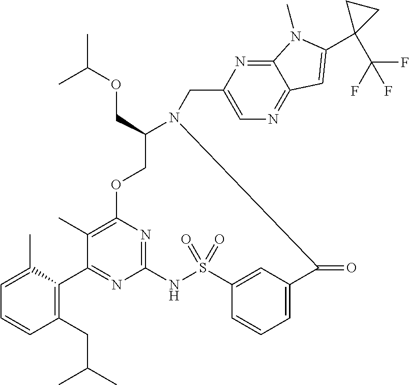 | | | +++ | +++ | |
| IV-77 | 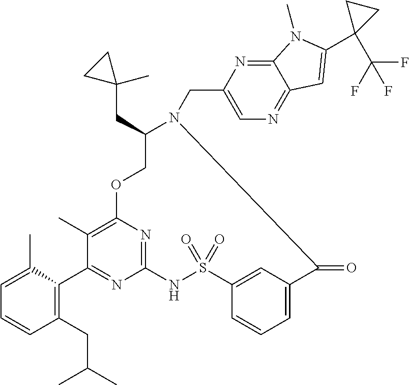 | | | ++ | +++ | |
| IV-78 | 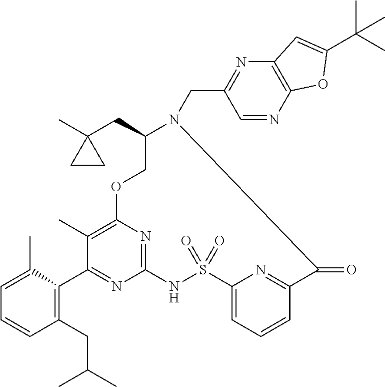 | | | | | |
| IV-79 | 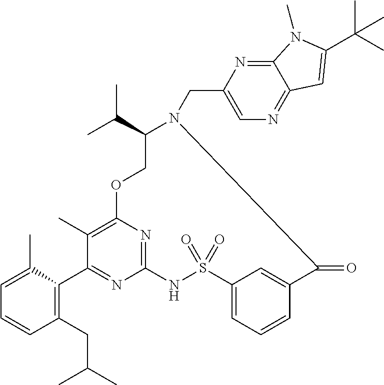 | | | +++ | +++ | |
| IV-80 | 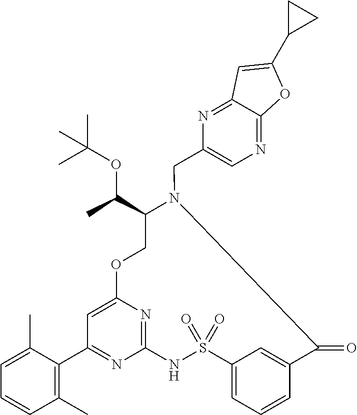 [SMILES: Cc1cccc(C)c1-c1cc2nc(n1)NS(=O)(=O)c1cccc(c1)C(=O)N(Cc1cnc3oc(C4CC4)cc3n1)[C@H]([C@@H](C)OC(C)(C)C)CO2] | | | ++ | +++ | |
| IV-81 | 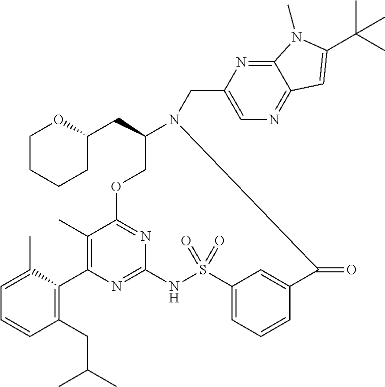 | | | +++ | +++ | |
| IV-82 | 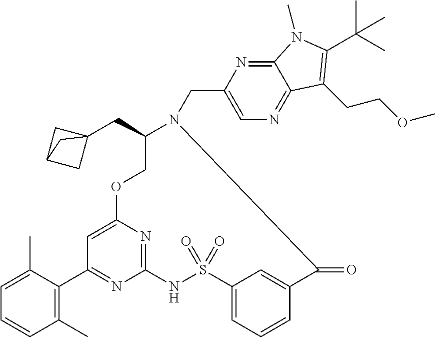 [SMILES: COCCc1c(C(C)(C)C)n(C)c2nc(CN3C(=O)c4cccc(c4)S(=O)(=O)Nc4nc(cc(-c5c(C)cccc5C)n4)OC[C@H]3CC34CC(C3)C4)cnc12] | | | +++ | +++ | |
| IV-83 | 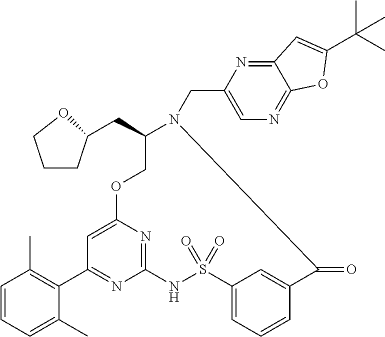 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@@H]1CCCO1)CO2] | | | ++ | +++ | |
| IV-84 | 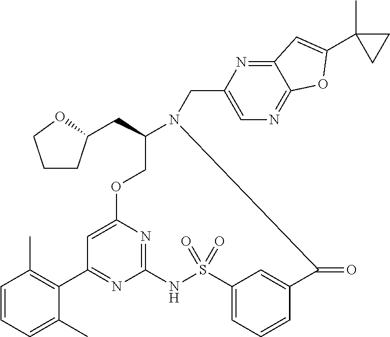 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@@H]1CCCO1)CO2] | | | ++ | +++ | |
| IV-85 | 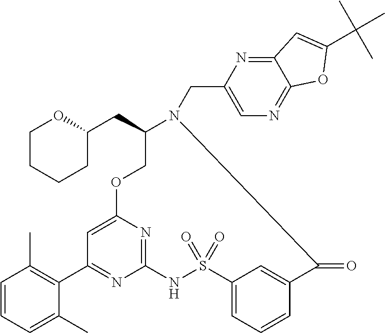 [SMILES: Cc1cccc(C)c1-c1cc2nc(n1)NS(=O)(=O)c1cccc(c1)C(=O)N(Cc1cnc3oc(C(C)(C)C)cc3n1)[C@H](C[C@@H]1CCCCO1)CO2] | | | ++ | +++ | |
| IV-86 | 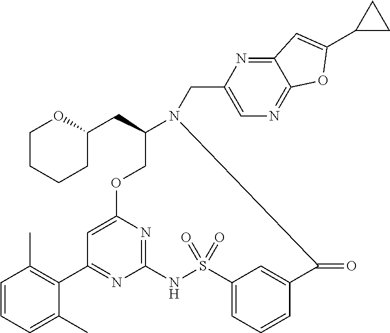 [SMILES: Cc1cccc(C)c1-c1cc2nc(n1)NS(=O)(=O)c1cccc(c1)C(=O)N(Cc1cnc3oc(C4CC4)cc3n1)[C@H](C[C@@H]1CCCCO1)CO2] | | | + | +++ | |
| IV-87 | 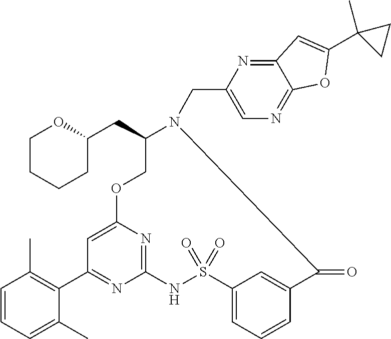 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@@H]1CCCCO1)CO2] | | | + | +++ | |
| IV-88 | 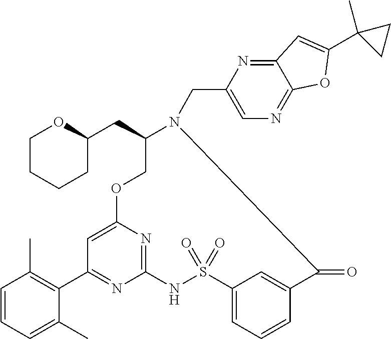 [SMILES: Cc1cccc(C)c1-c1cc2nc(n1)NS(=O)(=O)c1cccc(c1)C(=O)N(Cc1cnc3oc(C4(C)CC4)cc3n1)[C@H](C[C@H]1CCCCO1)CO2] | | | + | +++ | |
| IV-89 | 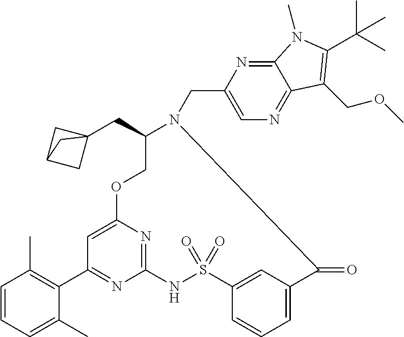 [SMILES: COCc1c(C(C)(C)C)n(C)c2nc(CN3C(=O)c4cccc(c4)S(=O)(=O)Nc4nc(cc(-c5c(C)cccc5C)n4)OC[C@H]3CC34CC(C3)C4)cnc12] | | | +++ | +++ | |
| IV-90 | 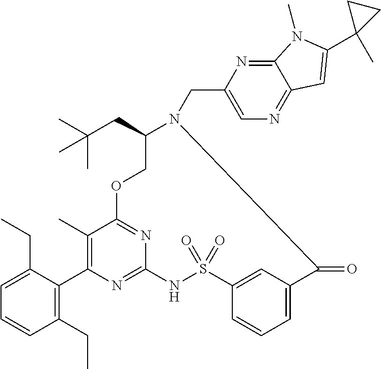 [SMILES: CCc1cccc(CC)c1-c1nc2nc(c1C)OC[C@@H](CC(C)(C)C)N(Cc1cnc3cc(C4(C)CC4)n(C)c3n1)C(=O)c1cccc(c1)S(=O)(=O)N2] | | | ++ | +++ | |
| IV-91 | 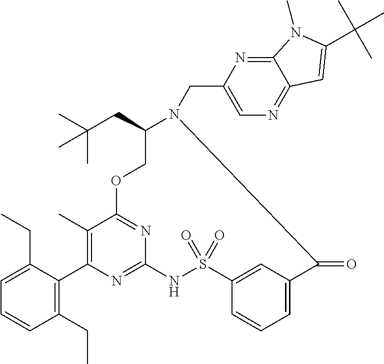 [SMILES: CCc1cccc(CC)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2] | | | +++ | +++ | |
| IV-92 | 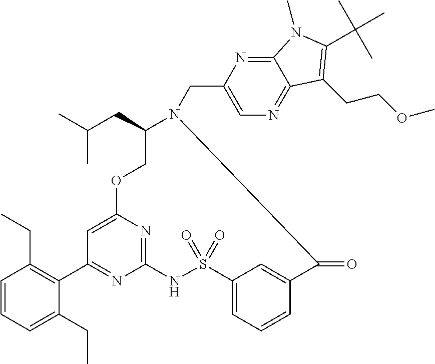 [SMILES: CCc1cccc(CC)c1-c1cc2nc(n1)NS(=O)(=O)c1cccc(c1)C(=O)N(Cc1cnc3c(CCOC)c(C(C)(C)C)n(C)c3n1)[C@H](CC(C)C)CO2] | | | +++ | +++ | |
| IV-93 | 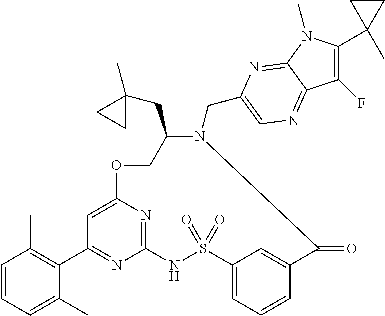 [SMILES: Cc1cccc(C)c1-c1cc2nc(n1)NS(=O)(=O)c1cccc(c1)C(=O)N(Cc1cnc3c(F)c(C4(C)CC4)n(C)c3n1)[C@H](CC1(C)CC1)CO2] | | | +++ | +++ | |
| IV-94 | 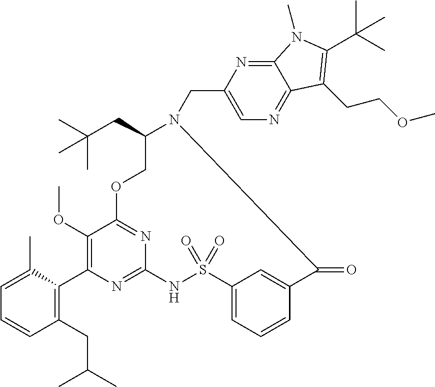 | +++ | +++ | +++ | +++ | |
| IV-95 | 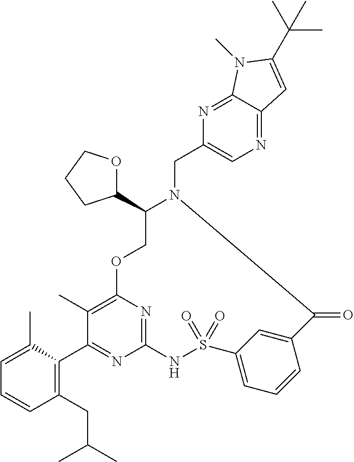 | | | +++ | +++ | |
| IV-96 | 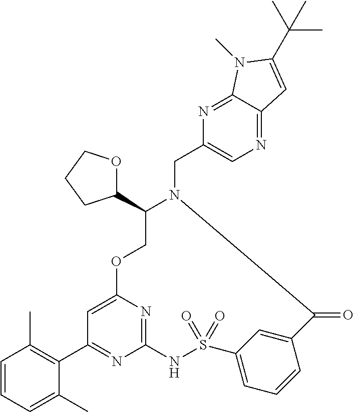 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](C1CCCO1)CO2] | | | +++ | +++ | |
| IV-97 | 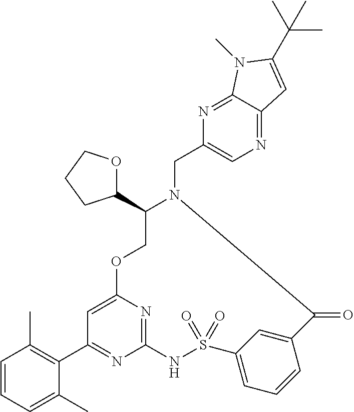 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](C1CCCO1)CO2] | | | +++ | +++ | |
| IV-98 | 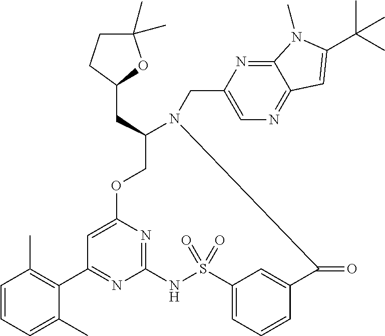 [SMILES: Cc1cccc(C)c1-c1cc2nc(n1)NS(=O)(=O)c1cccc(c1)C(=O)N(Cc1cnc3cc(C(C)(C)C)n(C)c3n1)[C@H](C[C@H]1CCC(C)(C)O1)CO2] | | | +++ | +++ | |
| IV-99 | 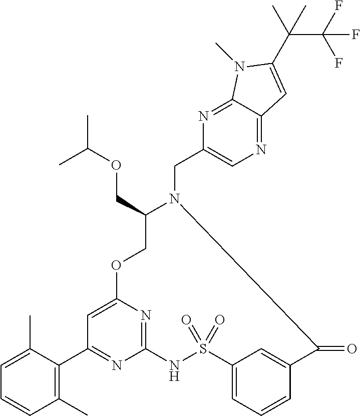 [SMILES: Cc1cccc(C)c1-c1cc2nc(n1)NS(=O)(=O)c1cccc(c1)C(=O)N(Cc1cnc3cc(C(C)(C)C(F)(F)F)n(C)c3n1)[C@H](COC(C)C)CO2] | | | ++ | +++ | |
| IV-100 | 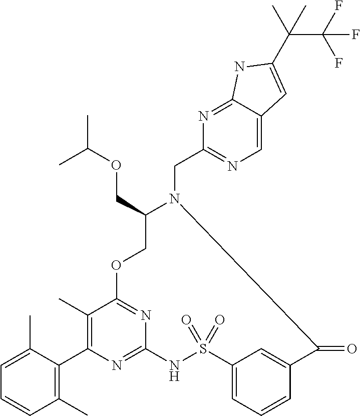 [SMILES: Cc1cccc(C)c1-c1nc2nc(c1C)OC[C@@H](COC(C)C)N(Cc1ncc3cc(C(C)(C)C(F)(F)F)[nH]c3n1)C(=O)c1cccc(c1)S(=O)(=O)N2] | | | +++ | +++ | |
| IV-101 | 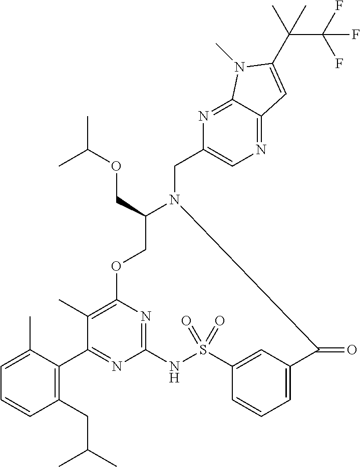 [SMILES: Cc1cccc(CC(C)C)c1-c1nc2nc(c1C)OC[C@@H](COC(C)C)N(Cc1cnc3cc(C(C)(C)C(F)(F)F)n(C)c3n1)C(=O)c1cccc(c1)S(=O)(=O)N2] | | | +++ | +++ | |
| IV-102 | 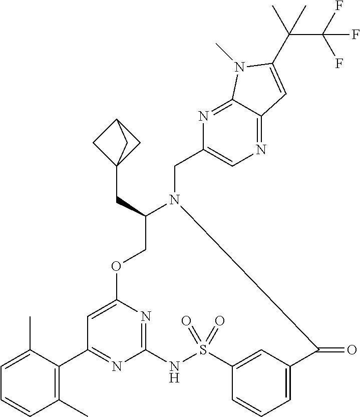 [SMILES: Cc1cccc(C)c1-c1cc2nc(n1)NS(=O)(=O)c1cccc(c1)C(=O)N(Cc1cnc3cc(C(C)(C)C(F)(F)F)n(C)c3n1)[C@H](CC13CC(C1)C3)CO2] | | | +++ | +++ | |
| IV-103 | 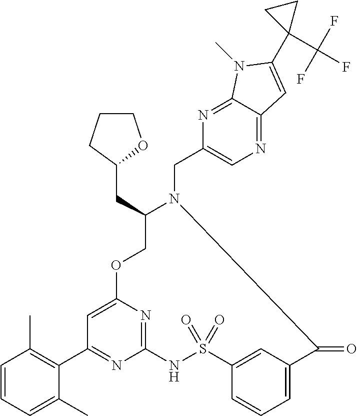 [SMILES: Cc1cccc(C)c1-c1cc2nc(n1)NS(=O)(=O)c1cccc(c1)C(=O)N(Cc1cnc3cc(C4(C(F)(F)F)CC4)n(C)c3n1)[C@H](C[C@@H]1CCCO1)CO2] | | | ++ | +++ | |
| IV-104 | 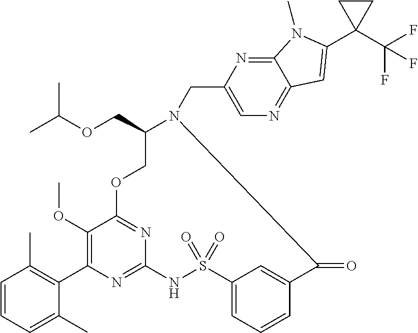 [SMILES: COc1c2nc(nc1-c1c(C)cccc1C)NS(=O)(=O)c1cccc(c1)C(=O)N(Cc1cnc3cc(C4(C(F)(F)F)CC4)n(C)c3n1)[C@H](COC(C)C)CO2] | | | ++ | +++ | |
| IV-105 | 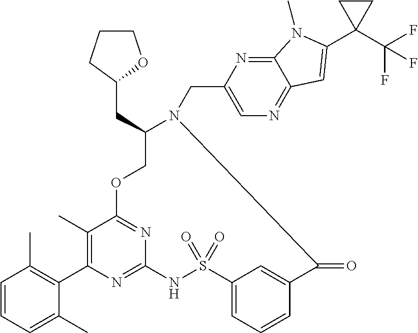 [SMILES: Cc1cccc(C)c1-c1nc2nc(c1C)OC[C@@H](C[C@@H]1CCCO1)N(Cc1cnc3cc(C4(C(F)(F)F)CC4)n(C)c3n1)C(=O)c1cccc(c1)S(=O)(=O)N2] | | | +++ | +++ | |
| IV-106 | 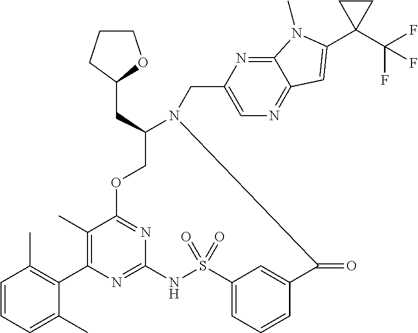 [SMILES: Cc1cccc(C)c1-c1nc2nc(c1C)OC[C@@H](C[C@H]1CCCO1)N(Cc1cnc3cc(C4(C(F)(F)F)CC4)n(C)c3n1)C(=O)c1cccc(c1)S(=O)(=O)N2] | | | +++ | +++ | |

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound of Formula I: 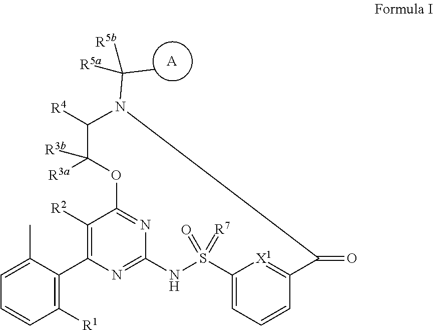
tautomers thereof, deuterated derivatives of those compounds and tautomers, and pharmaceutically acceptable salts of any of the foregoing, wherein:
Ring A is 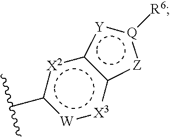
Q is selected from —C— and —N—;
W is selected from —CH—, —C(F)—, —C(CF₃)—, and —N—;
X¹, X², and X³ are each independently selected from —CH— and —N—;
Y is selected from —N—, —N(R^{y})—, —C(R^{y})—, and —O—, wherein
R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CH—, —O—, —S—, —S(O)—, —S(O)₂—, —N—, and —N^{z}, wherein
R^{z} is selected from hydrogen and C₁-C₈ alkyl;
R¹ is selected from: C₃-C₆ cycloalkyl, C₁-C₈ alkoxy, and C₁-C₈ alkyl (which may be optionally substituted with a group selected from C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
C₃-C₈ cycloalkyl (which may be optionally substituted with 1 to 2 groups selected from C₁-C₈ alkyl, C₁-C₈ alkoxy, halogen, and C₁-C₈ haloalkyl);
C₁-C₈ haloalkyl;
OC₃-C₇ cycloalkyl (which may be optionally substituted with 1 to 2 groups selected from C₁-C₈ alkyl, C₁-C₈ alkoxy, halogen, and C₁-C₈ haloalkyl);
phenyl (which may be optionally substituted with a group selected from C₁-C₈ alkyl, C₁-C₈ alkoxy, halogen, and C₁-C₈ haloalkyl);
C₁-C₈ alkoxy (which may be optionally substituted with a group selected from C₃-C₆ cycloalkyl and halogen);
4- to 6-membered heterocyclyl (which may be optionally substituted with 1 to 2 groups independently selected from halogen, C₁-C₈ haloalkyl, C₁-C₈ alkyl, and C₁-C₈ alkoxy); and
silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁶ is selected from halogen, 4- to 6-membered heterocyclyl, C₃-C₈ cycloalkyl (which may be optionally substituted with a group selected from C₁-C₈ alkyl, C₁-C₈ haloalkyl, and halogen); and C₁-C₈ alkyl (which may be optionally substituted with 1 to 2 groups independently selected from C₁-C₈ alkoxy, halogen, oxo, —OH, —NH₂, and —SO₂CH₃); and
R⁷ is selected from O, and NR, wherein
R is selected from hydrogen and C₁-C₈ alkyl;
with the proviso that wherein the compound of Formula I is not selected from: 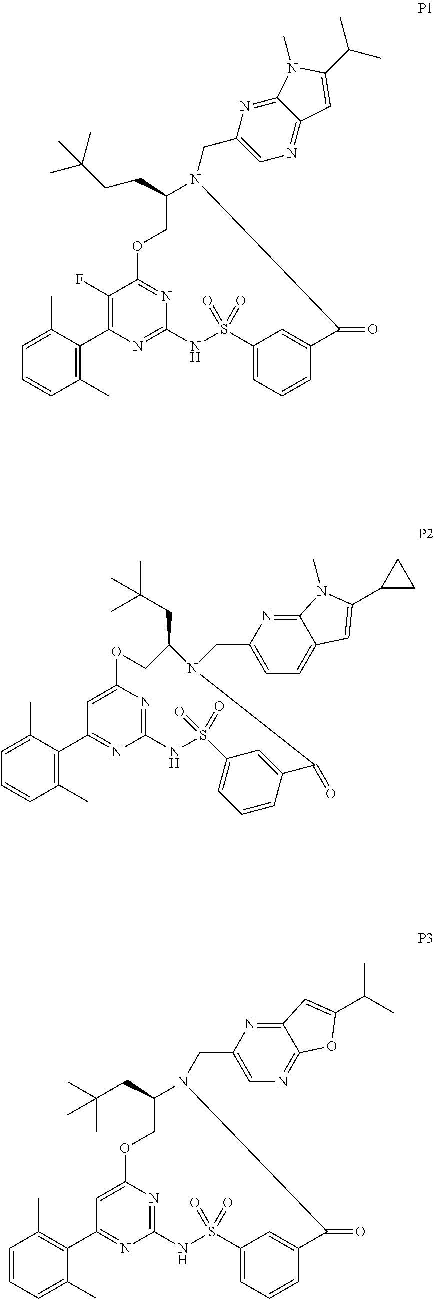 [SMILES: Cc1cccc(C)c1-c1nc2nc(c1F)OC[C@@H](CCC(C)(C)C)N(Cc1cnc3cc(C(C)C)n(C)c3n1)C(=O)c1cccc(c1)S(=O)(=O)N2 ; Cc1cccc(C)c1-c1cc2nc(n1)NS(=O)(=O)c1cccc(c1)C(=O)N(Cc1ccc3cc(C4CC4)n(C)c3n1)[C@H](CC(C)(C)C)CO2 ; Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2] 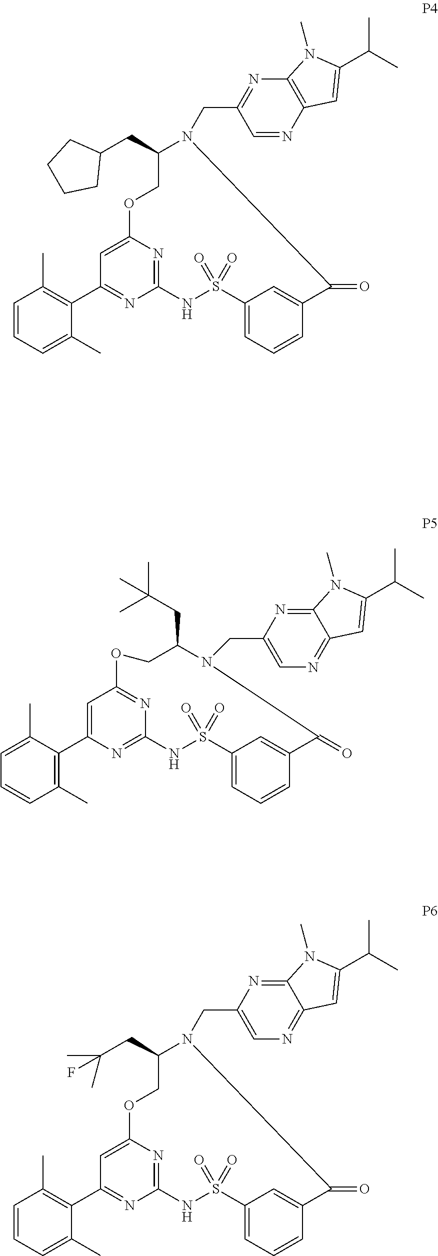 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)F)CO2] 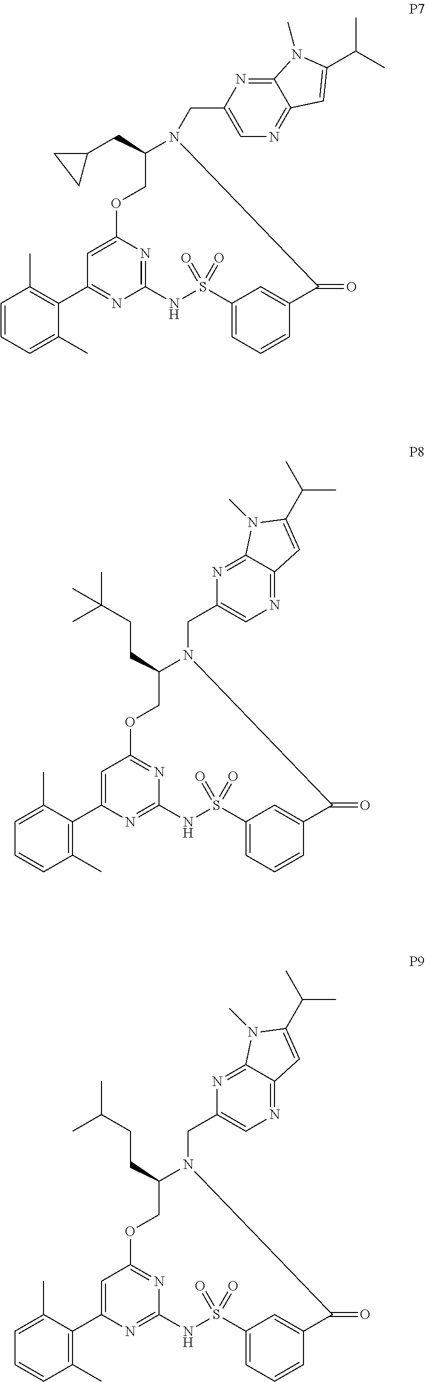 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CCC(C)C)CO2] 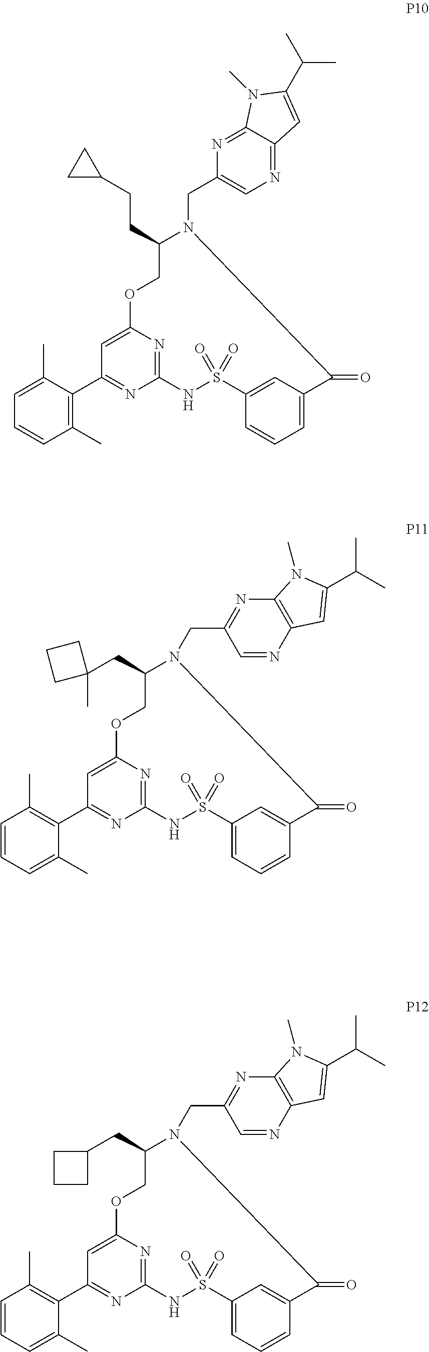 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1(C)CCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1CCC1)CO2] 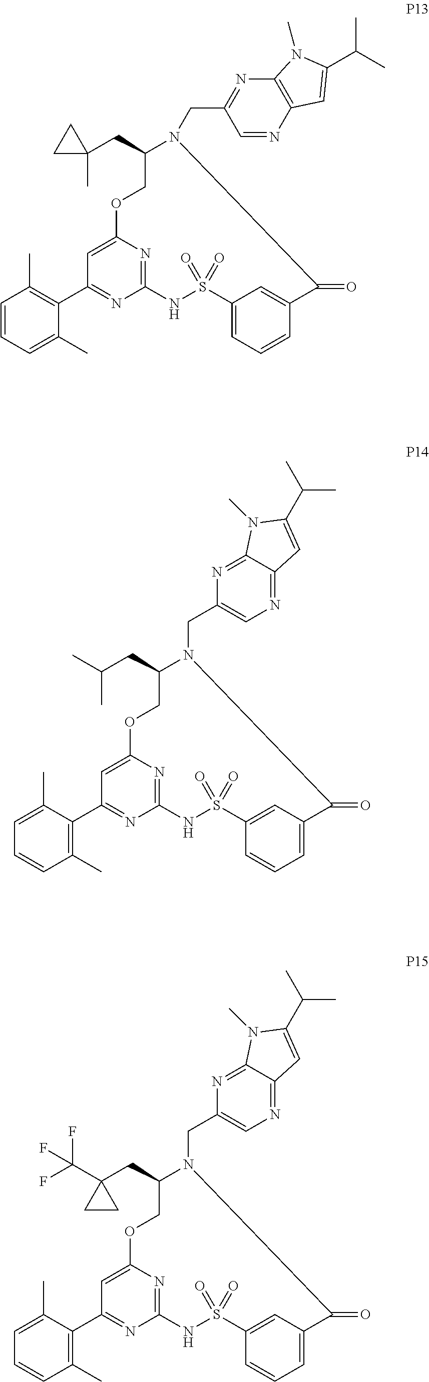 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1(C(F)(F)F)CC1)CO2] 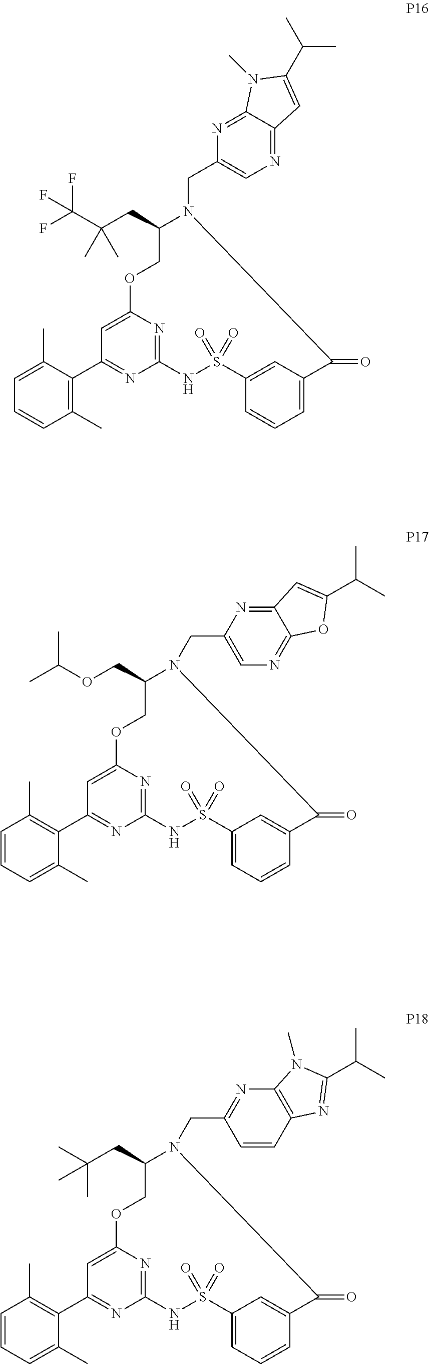 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C(F)(F)F)CO2 ; Cc1cccc(C)c1-c1cc2nc(n1)NS(=O)(=O)c1cccc(c1)C(=O)N(Cc1cnc3oc(C(C)C)cc3n1)[C@H](COC(C)C)CO2 ; Cc1cccc(C)c1-c1cc2nc(n1)NS(=O)(=O)c1cccc(c1)C(=O)N(Cc1ccc3nc(C(C)C)n(C)c3n1)[C@H](CC(C)(C)C)CO2] 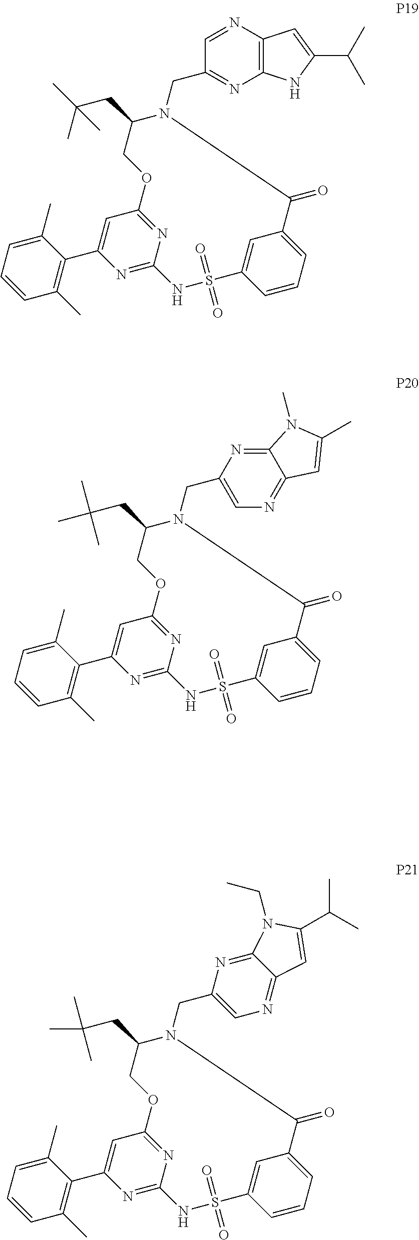 [SMILES: Cc1cccc(C)c1-c1cc2nc(n1)NS(=O)(=O)c1cccc(c1)C(=O)N(Cc1cnc3cc(C(C)C)[nH]c3n1)[C@H](CC(C)(C)C)CO2 ; Cc1cccc(C)c1-c1cc2nc(n1)NS(=O)(=O)c1cccc(c1)C(=O)N(Cc1cnc3cc(C)n(C)c3n1)[C@H](CC(C)(C)C)CO2 ; CCn1c(C(C)C)cc2ncc(CN3C(=O)c4cccc(c4)S(=O)(=O)Nc4nc(cc(-c5c(C)cccc5C)n4)OC[C@H]3CC(C)(C)C)nc21] 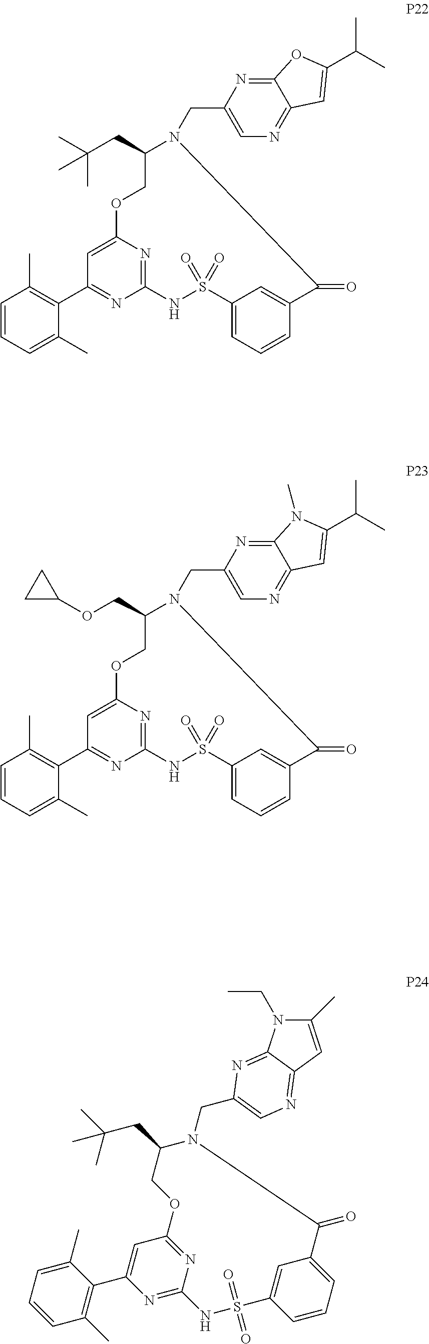 [SMILES: Cc1cccc(C)c1-c1cc2nc(n1)NS(=O)(=O)c1cccc(c1)C(=O)N(Cc1cnc3cc(C(C)C)oc3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](COC1CC1)CO2 ; CCn1c(C)cc2ncc(CN3C(=O)c4cccc(c4)S(=O)(=O)Nc4nc(cc(-c5c(C)cccc5C)n4)OC[C@H]3CC(C)(C)C)nc21] 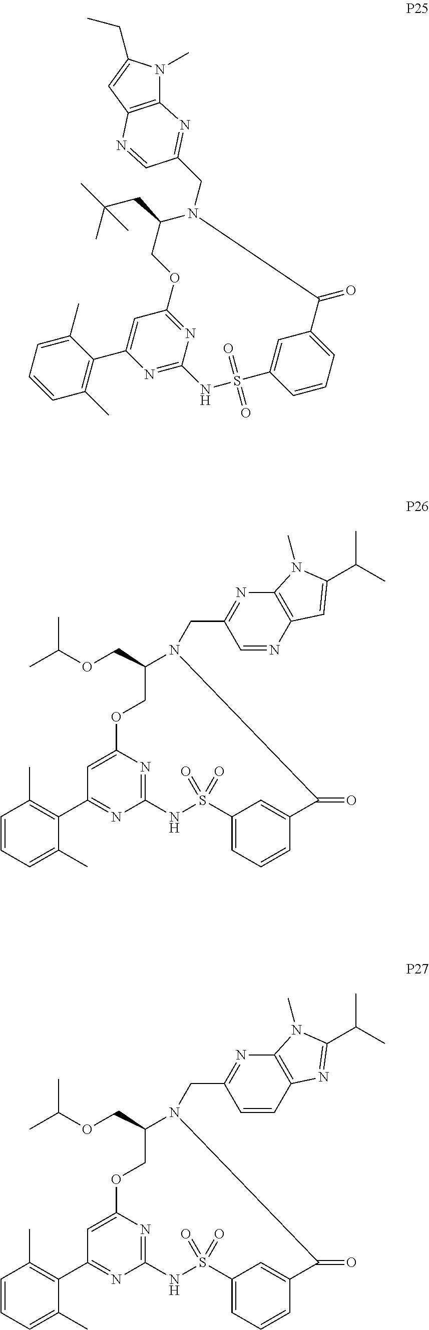 [SMILES: CCc1cc2ncc(CN3C(=O)c4cccc(c4)S(=O)(=O)Nc4nc(cc(-c5c(C)cccc5C)n4)OC[C@H]3CC(C)(C)C)nc2n1C ; Cc1cccc(C)c1-c1cc2nc(n1)NS(=O)(=O)c1cccc(c1)C(=O)N(Cc1cnc3cc(C(C)C)n(C)c3n1)[C@H](COC(C)C)CO2 ; Cc1cccc(C)c1-c1cc2nc(n1)NS(=O)(=O)c1cccc(c1)C(=O)N(Cc1ccc3nc(C(C)C)n(C)c3n1)[C@H](COC(C)C)CO2] 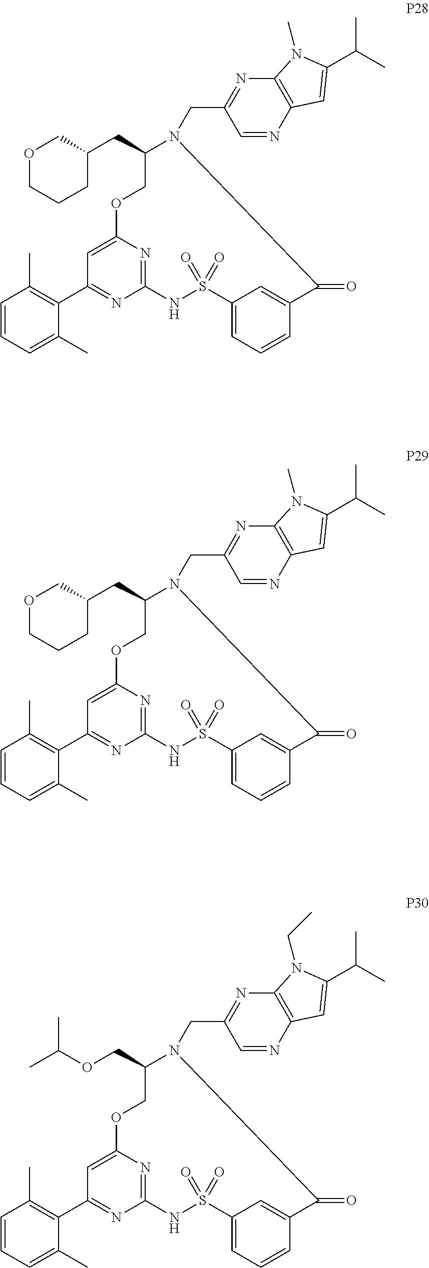 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; CCn1c(C(C)C)cc2ncc(CN3C(=O)c4cccc(c4)S(=O)(=O)Nc4nc(cc(-c5c(C)cccc5C)n4)OC[C@H]3COC(C)C)nc21] 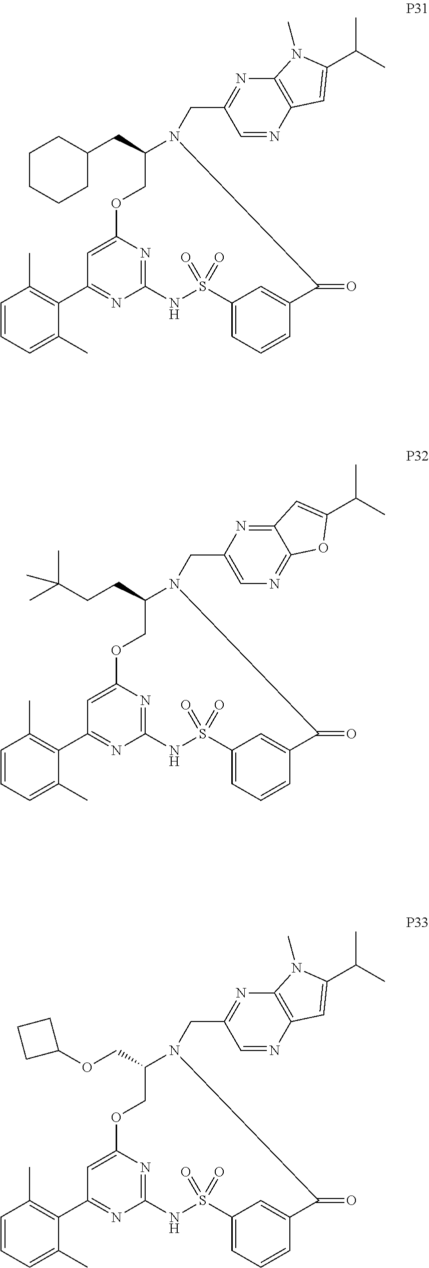 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CCC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2] 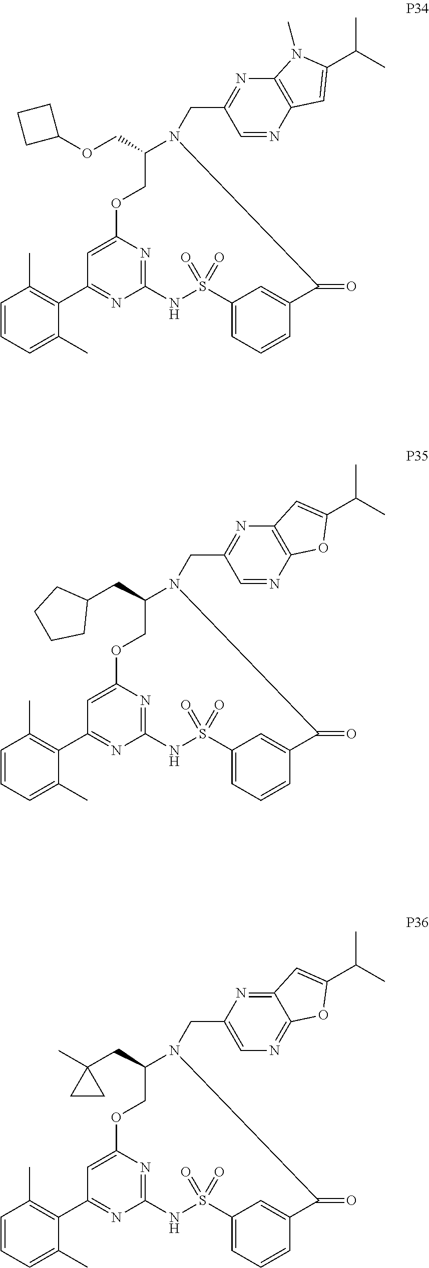 [SMILES: Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1CCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2]
and tautomers, deuterated derivatives, and pharmaceutically acceptable salts thereof.

2. The compound, tautomer, deuterated derivative, or salt of claim 1, selected from compounds of Formula Ia: 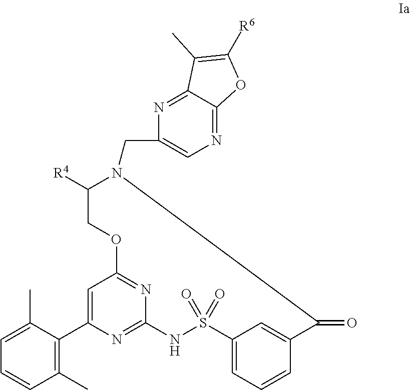
and tautomers thereof, or deuterated derivatives of the compounds and tautomers, and pharmaceutically acceptable salts of any of the foregoing,
and compounds of Formula Ia(i): 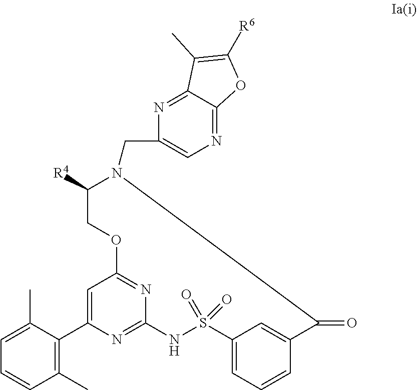
and tautomers thereof, or deuterated derivatives of the compounds and tautomers, and pharmaceutically acceptable salts of any of the foregoing.

3. The compound, tautomer, deuterated derivative, or salt of claim 1, selected from compounds of Formula Ib: 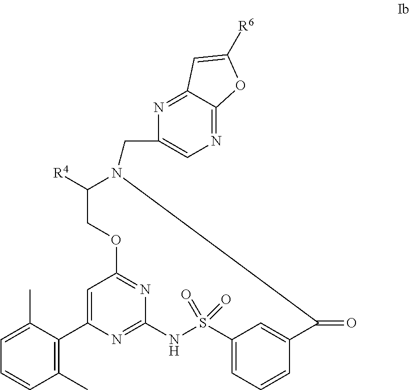
and tautomers thereof, or deuterated derivatives of the compounds and tautomers, and pharmaceutically acceptable salts of any of the foregoing,
and compounds of Formula Ib(i): 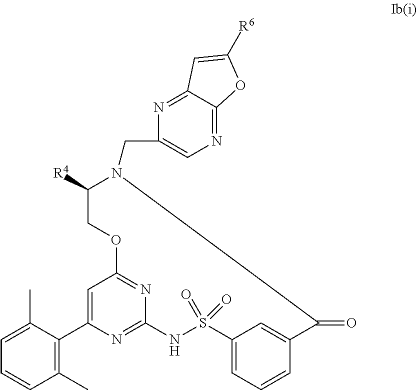
and tautomers thereof, or deuterated derivatives of the compounds and tautomers, and pharmaceutically acceptable salts of any of the foregoing.

4. The compound, tautomer, deuterated derivative, or salt of claim 1, selected from compounds of Formula Ic: 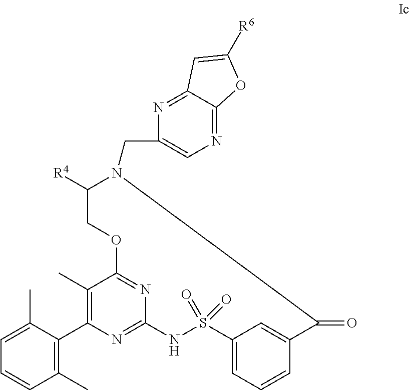
and tautomers thereof, or deuterated derivatives of the compounds and tautomers, and pharmaceutically acceptable salts of any of the foregoing,
and compounds of Formula Ic(i): 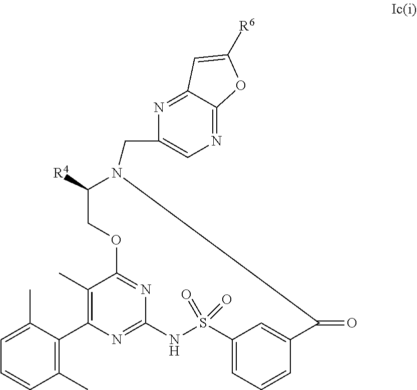
and tautomers thereof, or deuterated derivatives of the compounds and tautomers, and pharmaceutically acceptable salts of any of the foregoing.

5. The compound, tautomer, deuterated derivative, or salt of claim 1, selected from compounds of Formula Id: 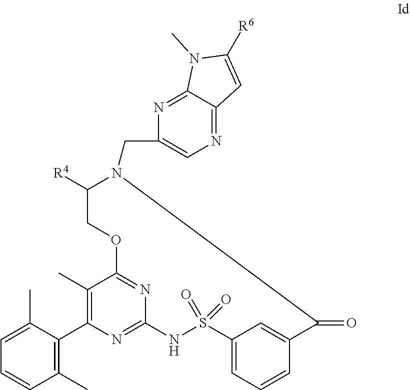
and tautomers thereof, or deuterated derivatives of the compounds and tautomers, and pharmaceutically acceptable salts of any of the foregoing
and compounds of Formula Id(i): 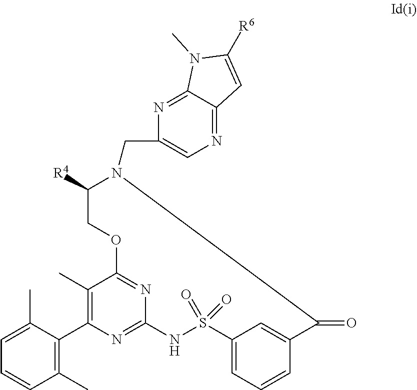
and tautomers thereof, or deuterated derivatives of the compounds and tautomers, and pharmaceutically acceptable salts of any of the foregoing, wherein R⁴ and R⁶ are as defined in claim 1.

6. The compound, tautomer, deuterated derivative, or salt of claim 1, selected from compounds of Formula Ie: 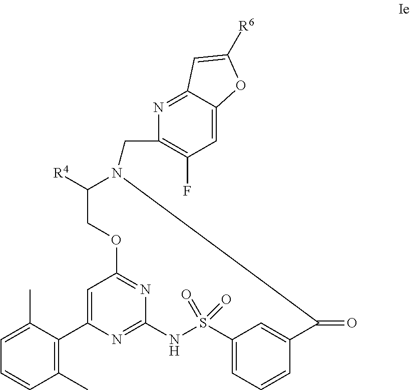
and tautomers thereof, or deuterated derivatives of the compounds and tautomers, and pharmaceutically acceptable salts of any of the foregoing
and compounds of Formula Ie(i): 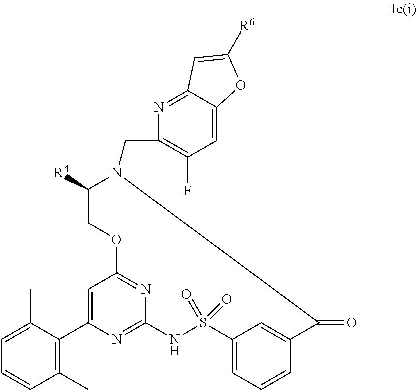
and tautomers thereof, or deuterated derivatives of the compounds and tautomers, and pharmaceutically acceptable salts of any of the foregoing, wherein R⁴ and R⁶ are as defined in claim 1.

7. The compound, tautomer, deuterated derivative, or salt of claim 1, selected from compounds of Formula If: 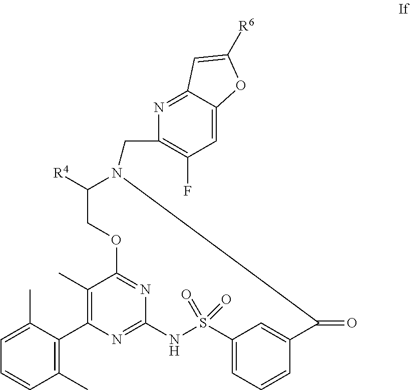
and tautomers thereof, or deuterated derivatives of the compounds and tautomers, and pharmaceutically acceptable salts of any of the foregoing,
and compounds of Formula If(i): 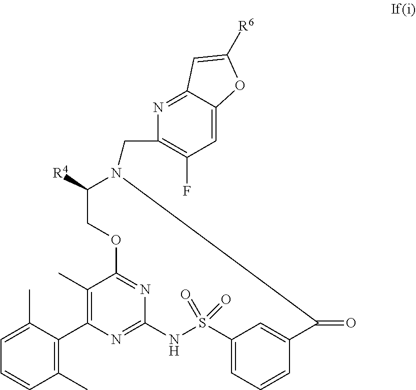
and tautomers thereof, or deuterated derivatives of the compounds and tautomers, and pharmaceutically acceptable salts of any of the foregoing, wherein R⁴ and R⁶ are as defined in claim 1.

8. A compound selected from Compounds I-1 to I-265, tautomers thereof, deuterated derivatives of the compounds and tautomers, and pharmaceutically acceptable salts of any of the foregoing.

9. The compound according to claim 8, selected from Compound I-4: 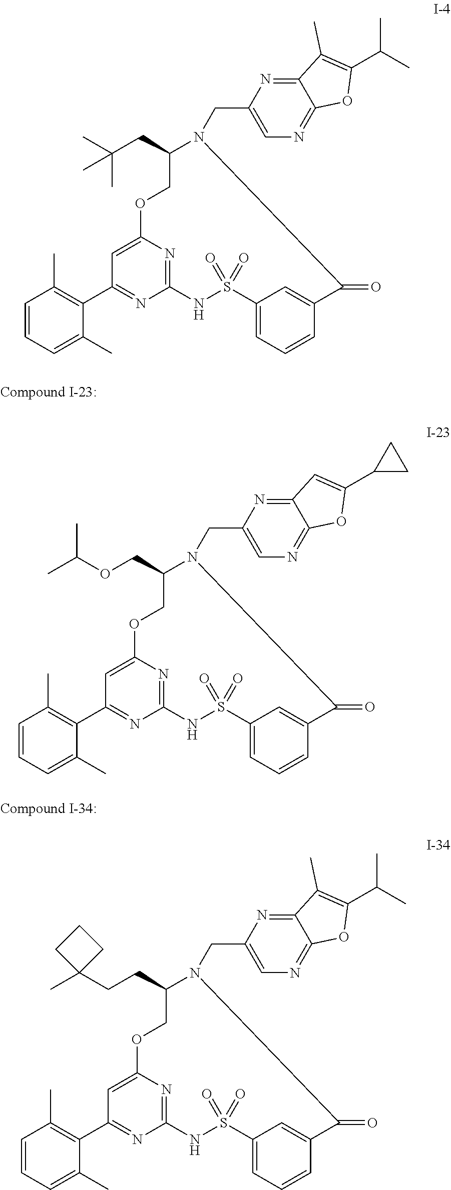 [SMILES: Cc1cccc(C)c1-c1cc2nc(n1)NS(=O)(=O)c1cccc(c1)C(=O)N(Cc1cnc3oc(C(C)C)c(C)c3n1)[C@H](CC(C)(C)C)CO2 ; Cc1cccc(C)c1-c1cc2nc(n1)NS(=O)(=O)c1cccc(c1)C(=O)N(Cc1cnc3oc(C4CC4)cc3n1)[C@H](COC(C)C)CO2 ; Cc1cccc(C)c1-c1cc2nc(n1)NS(=O)(=O)c1cccc(c1)C(=O)N(Cc1cnc3oc(C(C)C)c(C)c3n1)[C@H](CCC1(C)CCC1)CO2] 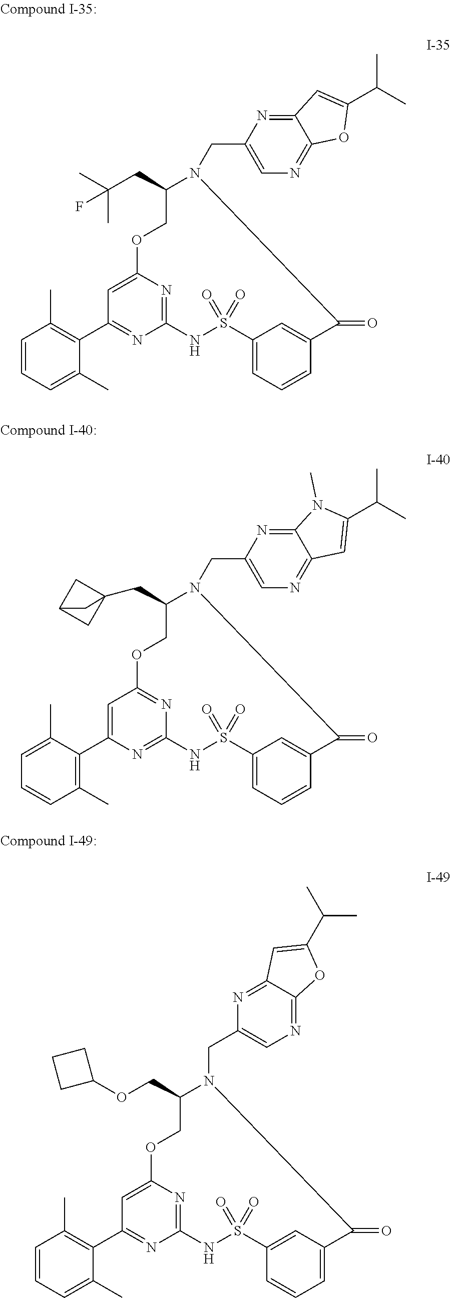 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)F)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC13CC(C1)C3)CO2 ; Cc1cccc(C)c1-c1cc2nc(n1)NS(=O)(=O)c1cccc(c1)C(=O)N(Cc1cnc3oc(C(C)C)cc3n1)[C@H](COC1CCC1)CO2] 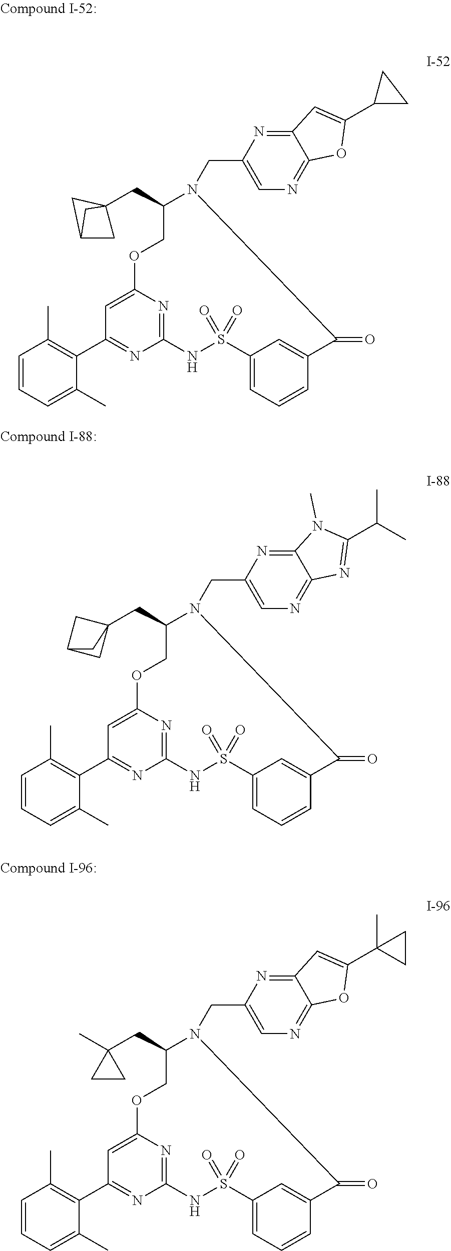 [SMILES: Cc1cccc(C)c1-c1cc2nc(n1)NS(=O)(=O)c1cccc(c1)C(=O)N(Cc1cnc3oc(C4CC4)cc3n1)[C@H](CC13CC(C1)C3)CO2 ; Cc1cccc(C)c1-c1cc2nc(n1)NS(=O)(=O)c1cccc(c1)C(=O)N(Cc1cnc3nc(C(C)C)n(C)c3n1)[C@H](CC13CC(C1)C3)CO2 ; Cc1cccc(C)c1-c1cc2nc(n1)NS(=O)(=O)c1cccc(c1)C(=O)N(Cc1cnc3oc(C4(C)CC4)cc3n1)[C@H](CC1(C)CC1)CO2] 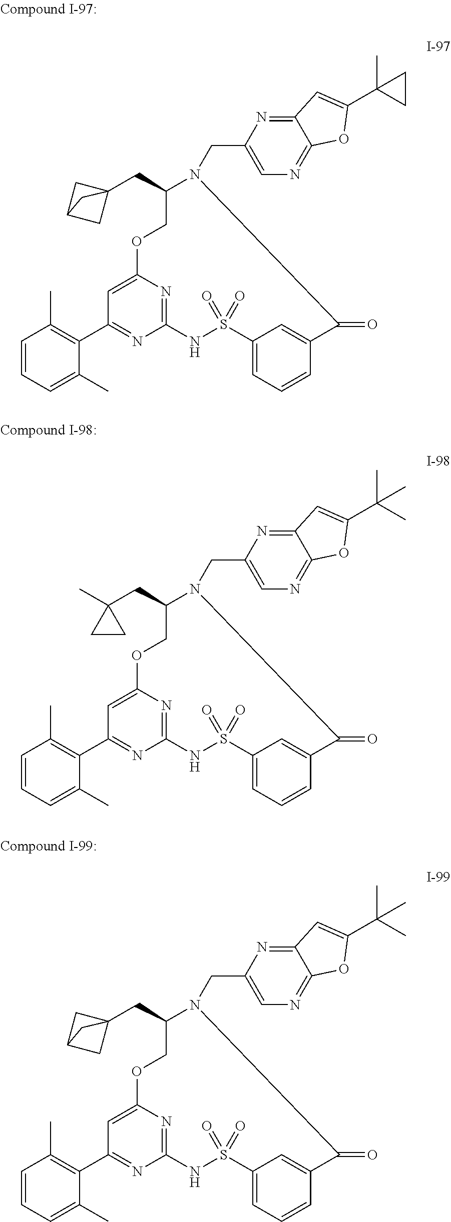 [SMILES: Cc1cccc(C)c1-c1cc2nc(n1)NS(=O)(=O)c1cccc(c1)C(=O)N(Cc1cnc3oc(C4(C)CC4)cc3n1)[C@H](CC13CC(C1)C3)CO2 ; Cc1cccc(C)c1-c1cc2nc(n1)NS(=O)(=O)c1cccc(c1)C(=O)N(Cc1cnc3oc(C(C)(C)C)cc3n1)[C@H](CC1(C)CC1)CO2 ; Cc1cccc(C)c1-c1cc2nc(n1)NS(=O)(=O)c1cccc(c1)C(=O)N(Cc1cnc3oc(C(C)(C)C)cc3n1)[C@H](CC13CC(C1)C3)CO2] 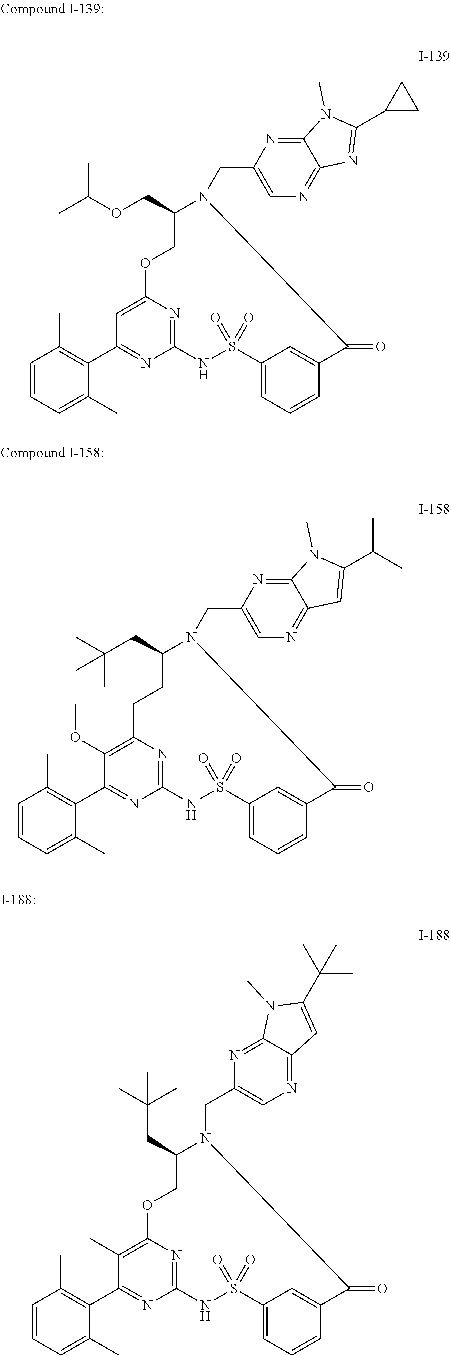 [SMILES: Cc1cccc(C)c1-c1cc2nc(n1)NS(=O)(=O)c1cccc(c1)C(=O)N(Cc1cnc3nc(C4CC4)n(C)c3n1)[C@H](COC(C)C)CO2 ; COc1c2nc(nc1-c1c(C)cccc1C)NS(=O)(=O)c1cccc(c1)C(=O)N(Cc1cnc3cc(C(C)C)n(C)c3n1)[C@H](CC(C)(C)C)CC2 ; Cc1cccc(C)c1-c1nc2nc(c1C)OC[C@@H](CC(C)(C)C)N(Cc1cnc3cc(C(C)(C)C)n(C)c3n1)C(=O)c1cccc(c1)S(=O)(=O)N2] 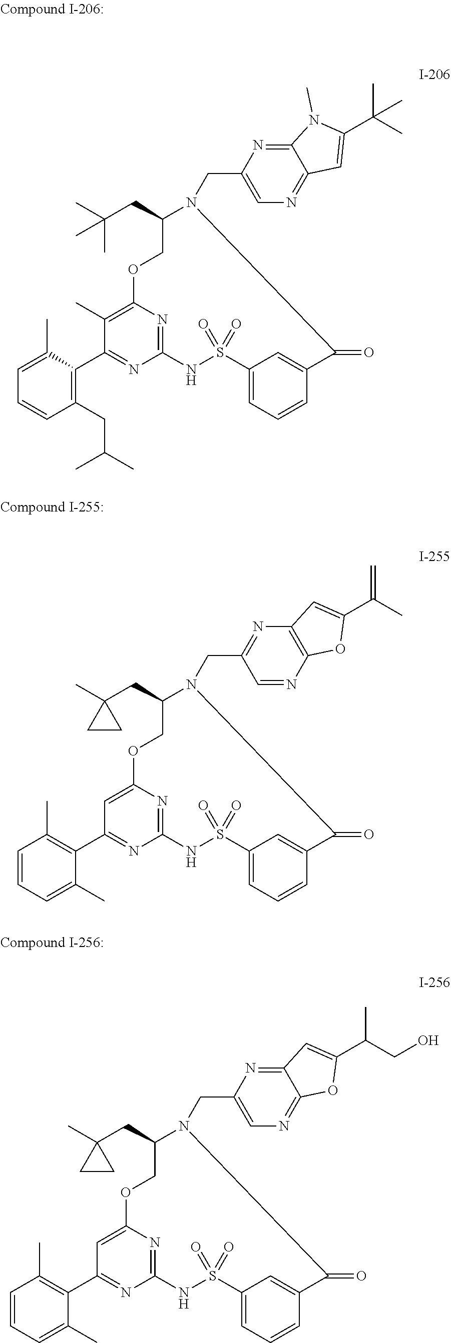
and tautomers thereof, deuterated derivatives of the compounds and tautomers, and pharmaceutically acceptable salts of any of the foregoing.

10. A compound of Formula II: 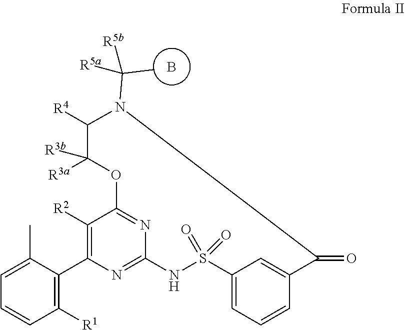
or a tautomer thereof, or a deuterated derivative of the compound or tautomer, or a pharmaceutically acceptable salt of any of the foregoing, wherein:
Ring B is a 6-membered heteroaryl, optionally substituted with 1 to 2 groups independently selected from
halogen
4- to 10-membered heterocyclyl (which may be optionally substituted with 1 to 3 groups independently selected from halogen, oxo, C₁-C₄ alkyl)
N(R^{x})₂, wherein R^{x} is independently selected from hydrogen, C₁-C₄ alkyl, C₃-C₆ cycloalkyl (which may be optionally substituted with a group selected from halogen, C₁-C₄ haloalkyl, and C₁-C₄ alkyl)
C₁-C₄ alkyl (optionally substituted with C₃-C₆ cycloalkyl (which may be further optionally substituted with a group selected from halogen, OH))
R¹ is selected from: C₃-C₆ cycloalkyl, C₁-C₆ alkoxy, and C₁-C₆ alkyl (which may be optionally substituted with a C₄-C₆ cycloalkyl);
R² is selected from: hydrogen, halogen, C₁-C₂ alkyl, C₁-C₄ haloalkyl, and C₁-C₂ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
C₃-C₈ cycloalkyl (which may be optionally substituted with 1 to 2 groups selected from C₁-C₈ alkyl, C₁-C₈ alkoxy, halogen, and C₁-C₈ haloalkyl);
C₁-C₈ haloalkyl;
phenyl (which may be optionally substituted with a group selected from C₁-C₈ alkyl, C₁-C₈ alkoxy, halogen, and C₁-C₈ haloalkyl);
C₁-C₈ alkoxy (which may be optionally substituted with a group selected from C₃-C₆ cycloalkyl and halogen);
4- to 6-membered heterocyclyl (which may be optionally substituted with 1 to 2 groups independently selected from halogen, C₁-C₈ haloalkyl, C₁-C₈ alkyl, and C₁-C₈ alkoxy); and
silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
and wherein the compound of Formula II is selected from Compounds II-1 to II-38 and tautomers thereof, deuterated derivatives of the compounds and tautomers, and pharmaceutically acceptable salts of any of the foregoing.

11. A compound of Formula III: 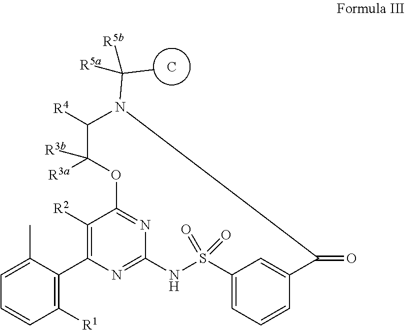
or a tautomer thereof, or a deuterated derivative of the compound or tautomer, or a pharmaceutically acceptable salt of any of the foregoing, wherein:
Ring C is selected from: 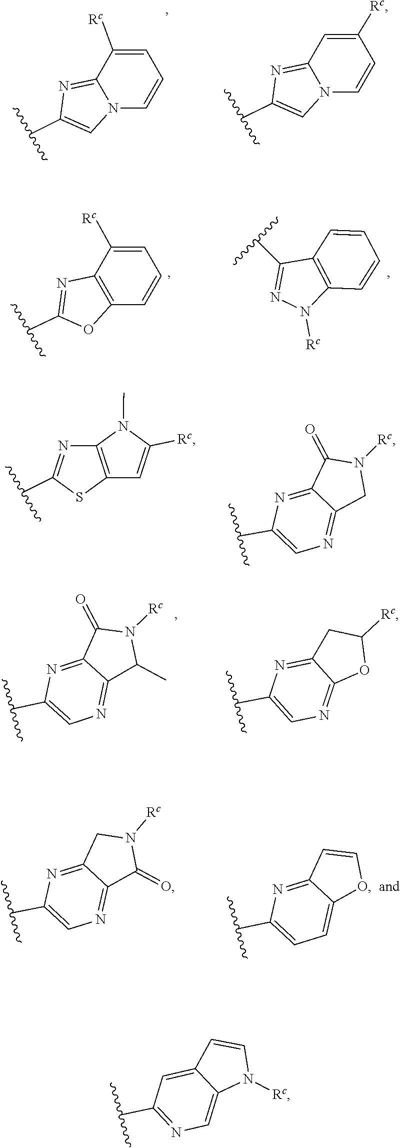
wherein
each R^{c} is independently selected from hydrogen, halogen, cyano, amino, C₁-C₄ alkyl (which may be optionally substituted with a group selected from —OH, halogen, and oxo), and C₃-C₆ alkenyl;
R¹ is selected from: C₃-C₆ cycloalkyl, C₁-C₆ alkoxy, and C₁-C₆ alkyl (which may be optionally substituted with a C₄-C₆ cycloalkyl);
R² is selected from: hydrogen, halogen, C₁-C₂ alkyl, C₁-C₄ haloalkyl, and C₁-C₂ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₆ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
C₃-C₈ cycloalkyl (which may be optionally substituted with 1 to 2 groups selected from C₁-C₈ alkyl, C₁-C₈ alkoxy, halogen, and C₁-C₈ haloalkyl);
C₁-C₈ haloalkyl;
phenyl (which may be optionally substituted with a group selected from C₁-C₈ alkyl, C₁-C₈ alkoxy, halogen, and C₁-C₈ haloalkyl);
C₁-C₈ alkoxy (which may be optionally substituted with a group selected from C₃-C₆ cycloalkyl and halogen);
4- to 6-membered heterocyclyl (which may be optionally substituted with 1 to 2 groups independently selected from halogen, C₁-C₈ haloalkyl, C₁-C₈ alkyl, and C₁-C₈ alkoxy); and
silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
and wherein the compound of Formula III is selected from Compounds III-1 to III-25 and tautomers thereof, deuterated derivatives of the compounds and tautomers, and pharmaceutically acceptable salts of any of the foregoing.

12. A compound of Formula IV: 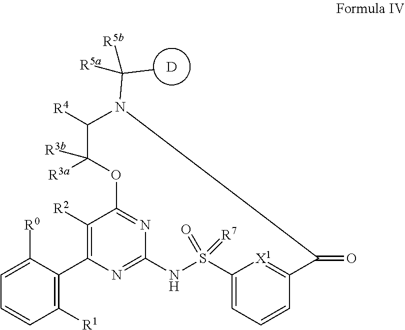
or a tautomer thereof, or a deuterated derivative of the compound or tautomer, or a pharmaceutically acceptable salt of any of the foregoing, wherein:
Ring D is 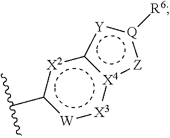
Q is selected from —C— and —N—;
W is selected from —CH—, —C(F)—, —C(CF₃)—, and —N—;
X¹, X², and X³ are each independently selected from —CH— and —N—;
X⁴ is selected from C and N;
Y is selected from —N—, —N(R^{y})—, —C(R^{y})—, and —O—, wherein
R^{y} is selected from hydrogen, halogen, C₁-C₈ haloalkyl, cyano, —NH₂, C₃-C₆ cycloalkyl, C₁-C₈ alkyl (which may be optionally substituted with a group selected from —OH and C₁-C₈ alkoxy), —NHC(O)OC₁-C₈ alkyl (which may be optionally substituted with a group selected from —OH and halogen);
Z is selected from —CR^{z}—, —O—, —S—, —S(O)—, —S(O)₂—, —N—, and —N^{z}, wherein
R^{z} is selected from hydrogen, halogen, and C₁-C₈ alkyl (which may be optionally substituted with C₁-C₈ alkoxy;
R⁰ is selected from C₁-C₂ alkyl;
R¹ is selected from: C₃-C₆ cycloalkyl, C₁-C₈ alkoxy, and C₁-C₈ alkyl (which may be optionally substituted with a group selected from C₁-C₈ alkoxy, C₄-C₆ cycloalkyl, C₅-C₆ aryl, 4- to 6-membered heterocyclyl, and 4- to 6-membered heteroaryl);
R² is selected from: hydrogen, halogen, C₁-C₈ alkyl, C₁-C₈ haloalkyl, and C₁-C₈ alkoxy;
R^{3a} and R^{3b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁴ is selected from:
C₃-C₈ cycloalkyl, which may be optionally substituted with 1 to 3 groups independently selected from halogen, C₁-C₈ haloalkyl, and C₁-C₈ alkyl; and
C₁-C₉ alkyl, which may be optionally substituted with 1 to 2 groups independently selected from:
—OH
C₃-C₈ cycloalkyl (which may be optionally substituted with 1 to 2 groups selected from C₁-C₈ alkyl, C₁-C₈ alkoxy, halogen, and C₁-C₈ haloalkyl);
C₁-C₈ haloalkyl;
—OC₃-C₇ cycloalkyl (which may be optionally substituted with 1 to 2 groups selected from C₁-C₈ alkyl, C₁-C₈ alkoxy, halogen, and C₁-C₈ haloalkyl);
phenyl (which may be optionally substituted with a group selected from C₁-C₈ alkyl, C₁-C₈ alkoxy, halogen, and C₁-C₈ haloalkyl);
C₁-C₈ alkoxy (which may be optionally substituted with a group selected from C₃-C₆ cycloalkyl, and phenyl; or which may be optionally substituted with 1 to 3 halogen atoms);
4- to 6-membered heterocyclyl (which may be optionally substituted with 1 to 2 groups independently selected from halogen, C₁-C₈ haloalkyl, C₁-C₈ alkyl, and C₁-C₈ alkoxy); and
silicon (which may be optionally substituted with 1 to 3 groups independently selected from C₁-C₈ alkyl, C₁-C₈ alkoxy, and C₁-C₈ haloalkyl);
R^{5a} and R^{5b} are independently selected from hydrogen, halogen, C₁-C₈ alkyl (which may be optionally substituted with 1-2 groups selected from halogen, hydroxyl, oxo, C₃-C₆ cycloalkyl, C₅-C₆ aryl, and 3-6 membered heterocyclyl), C₁-C₈ alkoxy (which may be optionally substituted with 1-2 groups selected from halogen, hydroxyl, oxo, C₃-C₆ cycloalkyl, C₅-C₆ aryl, and 3-6 membered heterocyclyl), or may be taken together to form a group selected from oxo and C₃-C₇ cycloalkyl (which may be optionally substituted with 1-2 groups selected from halogen, hydroxyl, oxo, C₁-C₄ alkyl, C₁-C₄ haloalkyl, C₃-C₆ cycloalkyl, C₅-C₆ aryl, and 3-6 membered heterocyclyl);
R⁶ is selected from hydrogen, cyano, halogen, 4- to 6-membered heterocyclyl, 5- to 6-membered heteroaryl (which may be optionally substituted with 1 to 2 groups selected from C₁-C₈ alkyl), C₃-C₅ cycloalkyl (which may be optionally substituted with a group selected from C₁-C₈ alkyl, C₁-C₈ haloalkyl, and halogen), phenyl, and C₁-C₈ alkyl (which may be optionally substituted with 1 to 2 groups independently selected from C₁-C₈ alkoxy, C₁-C₈ haloalkyl, halogen, oxo, —OH, —NH₂, and —SO₂CH₃); and
R⁷ is selected from O, and NR, wherein
R is selected from hydrogen and C₁-C₈ alkyl
with the proviso that wherein the compound of Formula IV is not selected from: 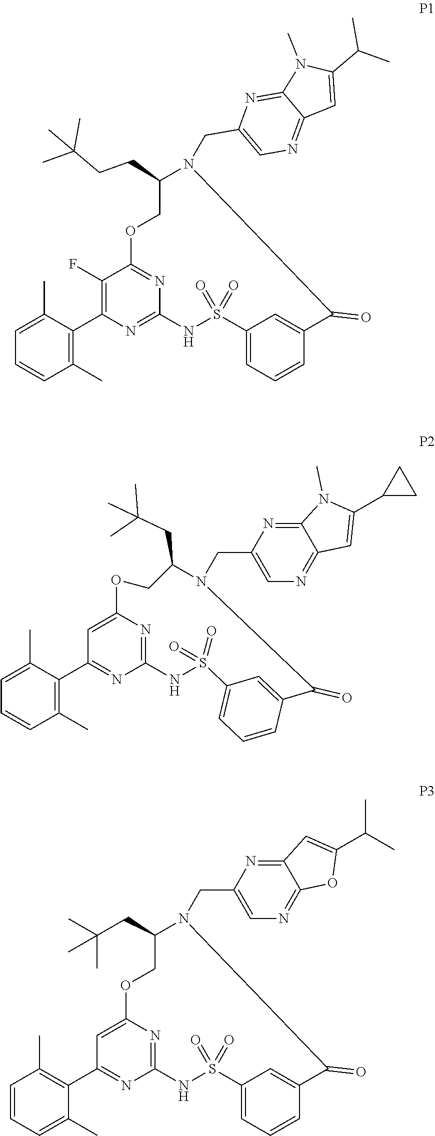 [SMILES: Cc1cccc(C)c1-c1nc2nc(c1F)OC[C@@H](CCC(C)(C)C)N(Cc1cnc3cc(C(C)C)n(C)c3n1)C(=O)c1cccc(c1)S(=O)(=O)N2 ; Cc1cccc(C)c1-c1cc2nc(n1)NS(=O)(=O)c1cccc(c1)C(=O)N(Cc1cnc3cc(C4CC4)n(C)c3n1)[C@H](CC(C)(C)C)CO2 ; Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2] 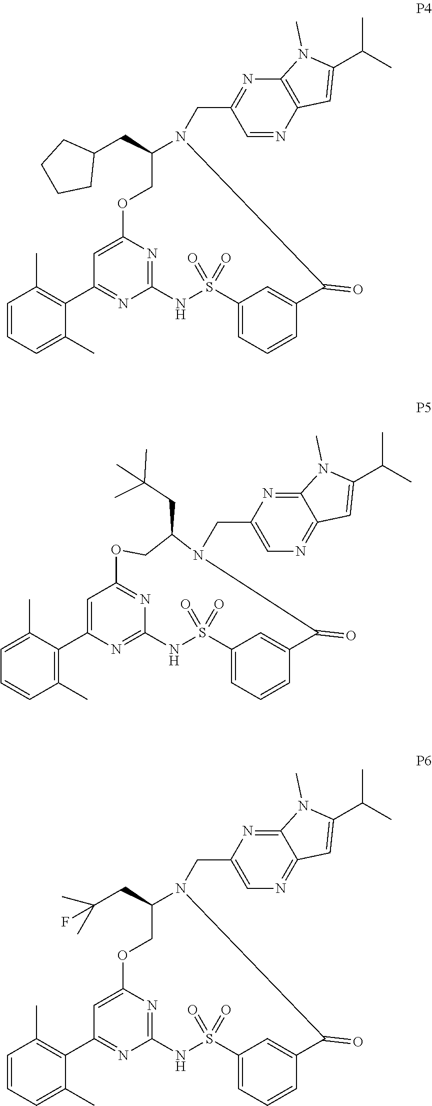 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)(C)F)CO2] 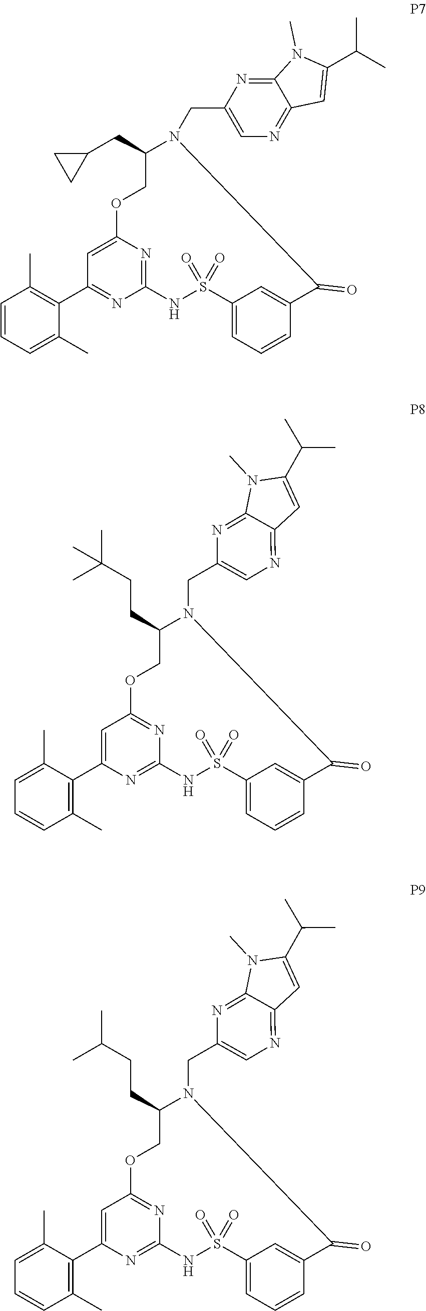 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CCC(C)C)CO2] 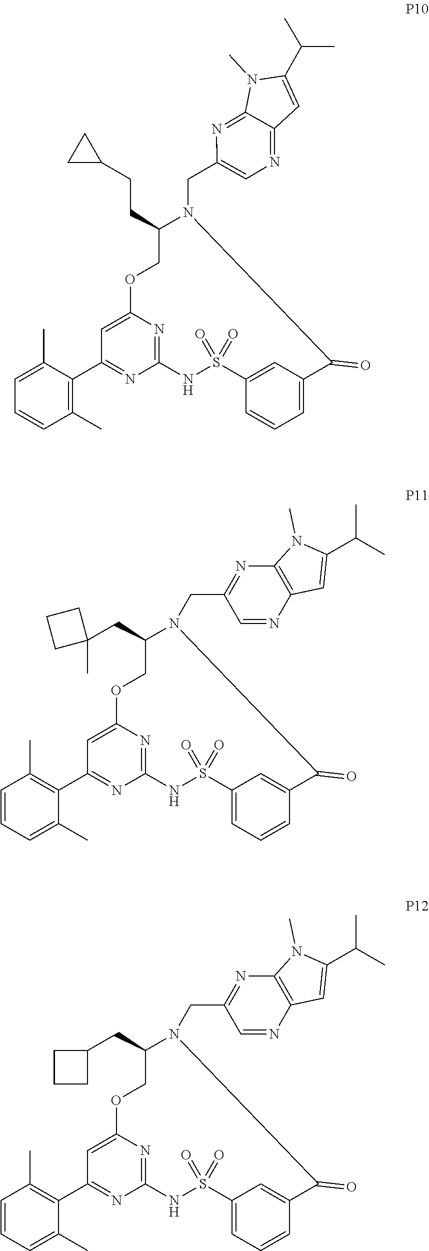 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1(C)CCC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1CCC1)CO2] 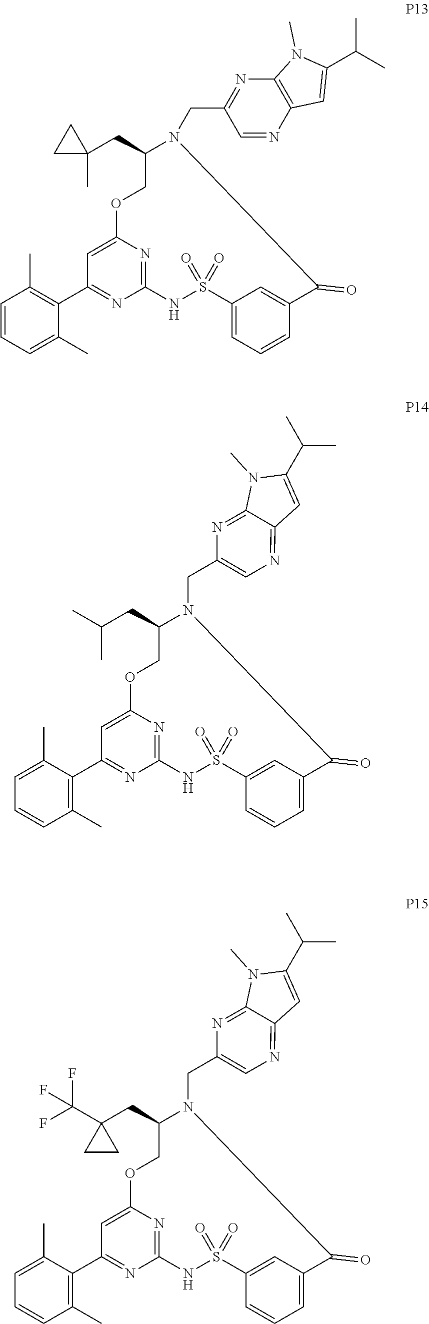 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](CC1(C(F)(F)F)CC1)CO2] 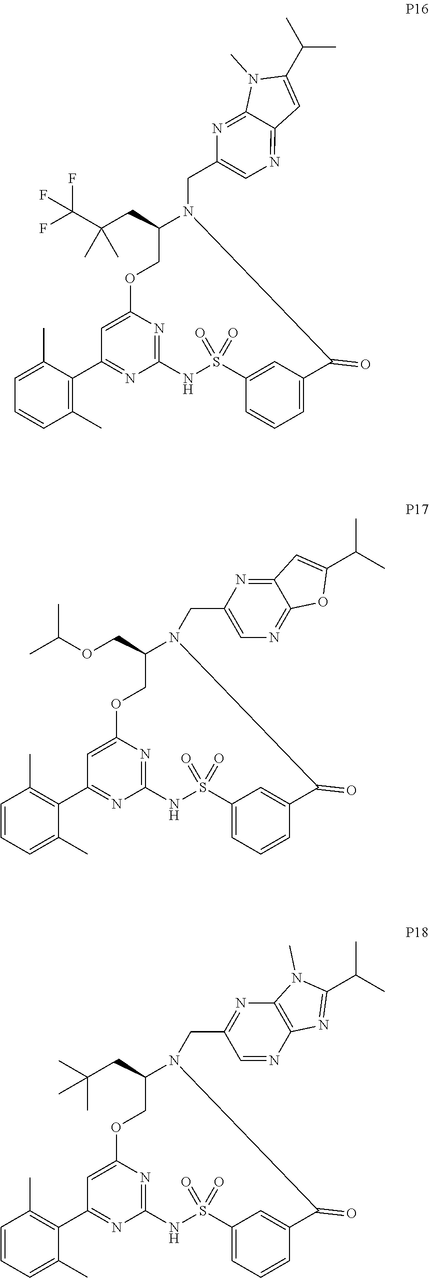 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C(F)(F)F)CO2 ; Cc1cccc(C)c1-c1cc2nc(n1)NS(=O)(=O)c1cccc(c1)C(=O)N(Cc1cnc3oc(C(C)C)cc3n1)[C@H](COC(C)C)CO2 ; Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CO2] 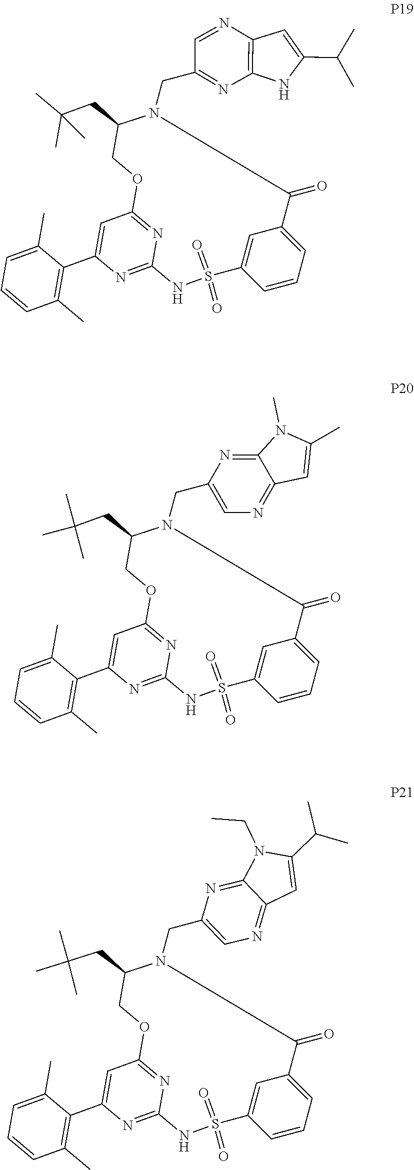 [SMILES: Cc1cccc(C)c1-c1cc2nc(n1)NS(=O)(=O)c1cccc(c1)C(=O)N(Cc1cnc3cc(C(C)C)[nH]c3n1)[C@H](CC(C)(C)C)CO2 ; Cc1cccc(C)c1-c1cc2nc(n1)NS(=O)(=O)c1cccc(c1)C(=O)N(Cc1cnc3cc(C)n(C)c3n1)[C@H](CC(C)(C)C)CO2 ; CCn1c(C(C)C)cc2ncc(CN3C(=O)c4cccc(c4)S(=O)(=O)Nc4nc(cc(-c5c(C)cccc5C)n4)OC[C@H]3CC(C)(C)C)nc21] 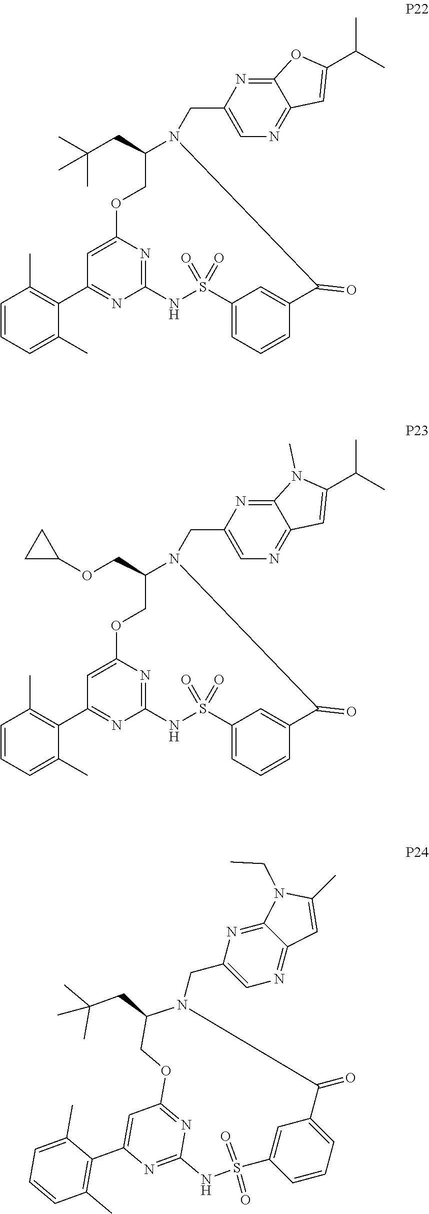 [SMILES: Cc1cccc(C)c1-c1cc2nc(n1)NS(=O)(=O)c1cccc(c1)C(=O)N(Cc1cnc3cc(C(C)C)oc3n1)[C@H](CC(C)(C)C)CO2 ; Cc1cccc(C)c1-c1cc2nc(n1)NS(=O)(=O)c1cccc(c1)C(=O)N(Cc1cnc3cc(C(C)C)n(C)c3n1)[C@H](COC1CC1)CO2 ; CCn1c(C)cc2ncc(CN3C(=O)c4cccc(c4)S(=O)(=O)Nc4nc(cc(-c5c(C)cccc5C)n4)OC[C@H]3CC(C)(C)C)nc21] 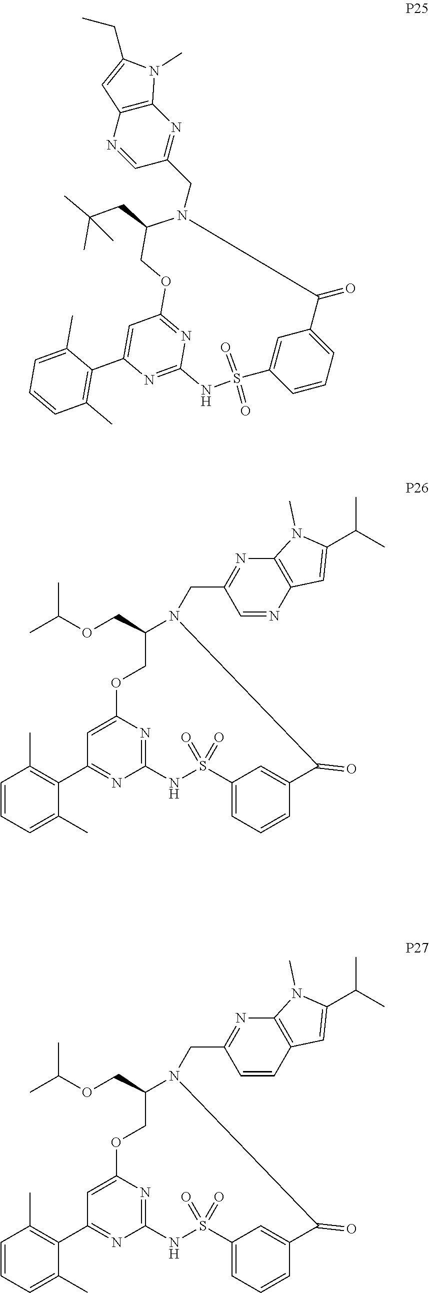 [SMILES: CCc1cc2ncc(CN3C(=O)c4cccc(c4)S(=O)(=O)Nc4nc(cc(-c5c(C)cccc5C)n4)OC[C@H]3CC(C)(C)C)nc2n1C ; Cc1cccc(C)c1-c1cc2nc(n1)NS(=O)(=O)c1cccc(c1)C(=O)N(Cc1cnc3cc(C(C)C)n(C)c3n1)[C@H](COC(C)C)CO2 ; Cc1cccc(C)c1-c1cc2nc(n1)NS(=O)(=O)c1cccc(c1)C(=O)N(Cc1ccc3cc(C(C)C)n(C)c3n1)[C@H](COC(C)C)CO2] 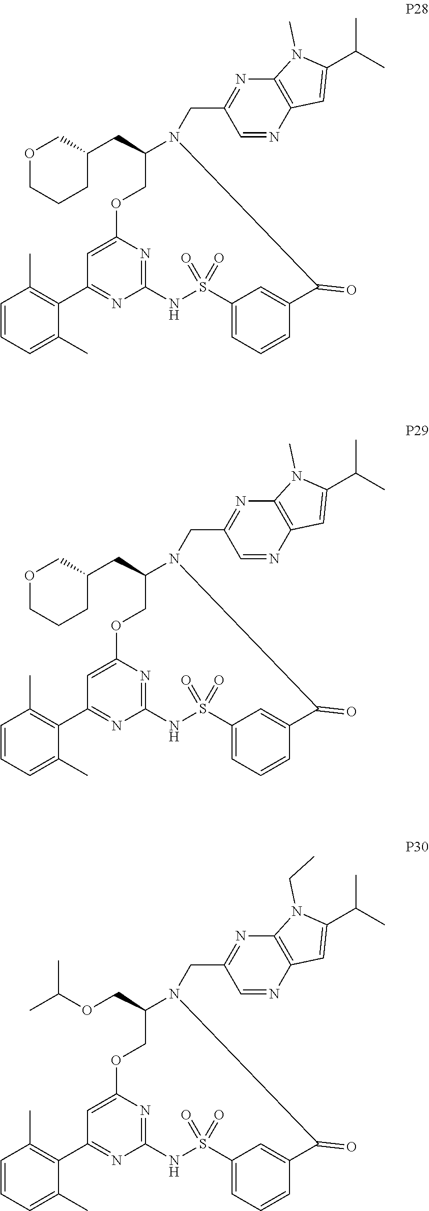 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2 ; CCn1c(C(C)C)cc2ncc(CN3C(=O)c4cccc(c4)S(=O)(=O)Nc4nc(cc(-c5c(C)cccc5C)n4)OC[C@H]3COC(C)C)nc21] 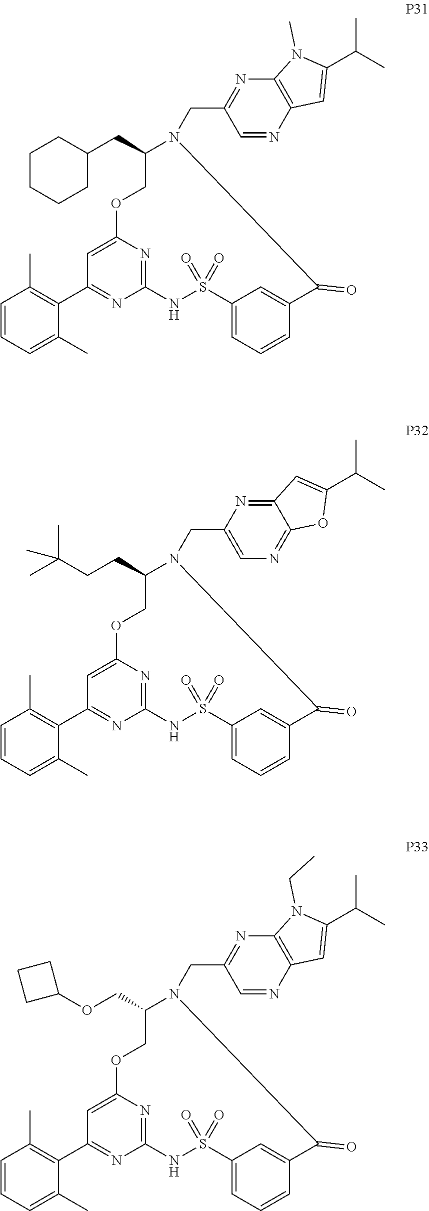 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CCC(C)(C)C)CO2 ; CCn1c(C(C)C)cc2ncc(CN3C(=O)c4cccc(c4)S(=O)(=O)Nc4nc(cc(-c5c(C)cccc5C)n4)OC[C@@H]3COC3CCC3)nc21] 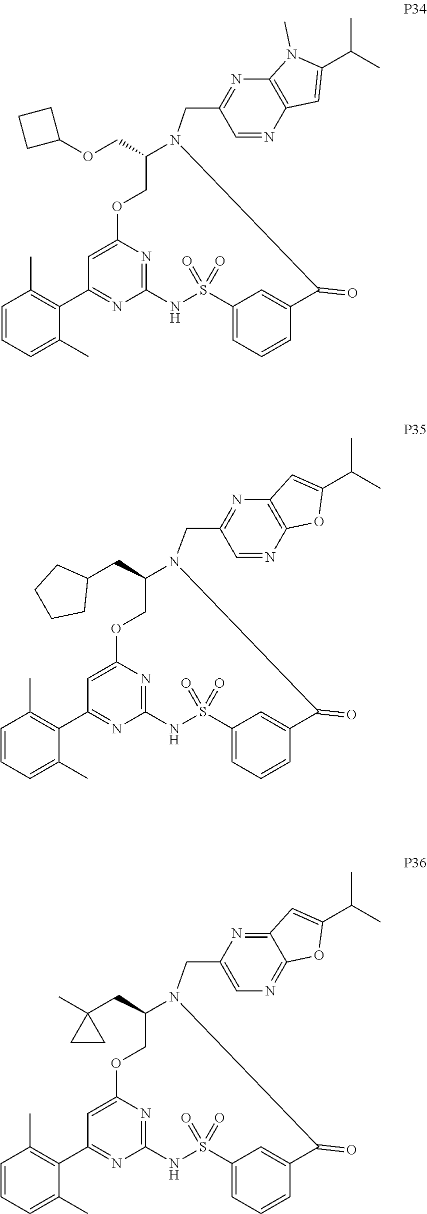 [SMILES: Cc1cccc(C)c1-c1cc2nc(n1)NS(=O)(=O)c1cccc(c1)C(=O)N(Cc1cnc3cc(C(C)C)n(C)c3n1)[C@@H](COC1CCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1CCCC1)CO2 ; Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2]
and tautomers, deuterated derivatives, and pharmaceutically acceptable salts thereof.

13. A compound selected from Compounds IV-1 to IV-106, tautomers thereof, deuterated derivatives of the compounds and tautomers, and pharmaceutically acceptable salts of any of the foregoing.

14. A pharmaceutical composition comprising a compound, tautomer, deuterated derivative, or pharmaceutically acceptable salt according to any one of claims 1 to 13 and a pharmaceutical carrier.

15. A method of treating cystic fibrosis comprising administering a compound, tautomer, deuterated derivative, or pharmaceutically acceptable salt according to any one of claims 1 to 13.

16. A compound, tautomer, deuterated derivative, or pharmaceutically acceptable salt according to any one of claims 1 to 13 for use in treating cystic fibrosis.

17. Use of compound, tautomer, deuterated derivative, or pharmaceutically acceptable salt according to any one of claims 1 to 13 in the manufacture of a medicament for treating cystic fibrosis.
